(19) United States
(12) Patent Application Publication
(10) Pub. No.: US 2023/0390404 A1
(43) Pub. Date: Dec. 7, 2023

(54) TETRAHYDROISOQUINOLINE HETEROBIFUNCTIONAL BCL-XL DEGRADERS

(71) Applicant: Treeline Biosciences, Inc., Stamford, CT (US)

(72) Inventors: Evan J. Horn, San Diego, CA (US); Joshua D. Hansen, San Diego, CA (US); Matthew D. Alexander, San Diego, CA (US); Fei Huang, La Mesa, CA (US); Mark A. Nagy, Encinitas, CA (US)

(21) Appl. No.: 18/143,454

(22) Filed: May 4, 2023

Related U.S. Application Data
(60) Provisional application No. 63/339,253, filed on May 6, 2022.
(60) Provisional application No. 63/398,783, filed on Aug. 17, 2022.
(60) Provisional application No. 63/429,862, filed on Dec. 2, 2022.
(60) Provisional application No. 63/449,756, filed on Mar. 3, 2023.
(60) Provisional application No. 63/454,545, filed on Mar. 24, 2023.
(60) Provisional application No. 63/457,997, filed on Apr. 7, 2023.

(51) Int. Cl. A61K 47/55; A61P 35/00; A61K 47/54

(52) U.S. Cl. CPC A61K 47/55; A61P 35/00; A61K 47/545

(57) ABSTRACT

This disclosure provides compounds of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (1-H-2), or (I-H-3))) or Formula (II) (e.g., (II-a)), or a pharmaceutically acceptable salt thereof that induce degradation of a BCL-X_{L} protein. These compounds are useful, for example, for treating a cancer in a subject (e.g., a human). This disclosure also provides compositions containing the compounds provided herein as well as methods of using and making the same.

### CROSS-REFERENCE TO RELATED APPLICATIONS

This application claims priority to U.S. Provisional Application Ser. No. 63/457,997, filed Apr. 7, 2023; 63/454,545, filed Mar. 24, 2023; 63/449,756, filed Mar. 3, 2023; 63/429,862, filed Dec. 2, 2022; 63/398,783, filed Aug. 17, 2022; and 63/339,253, filed May 6, 2022; each of which is incorporated by reference in its entirety herein.

### TECHNICAL FIELD

This disclosure provides compounds of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or pharmaceutically acceptable salts thereof, that induce degradation of a BCL-X_{L} protein. These compounds are useful, for example, for treating cancer in a subject (e.g., a human). This disclosure also provides compositions containing the compounds provided herein as well as methods of using and making the same.

### BACKGROUND

The BCL-2 family of proteins is involved in the regulation of cell apoptosis and includes proteins that are pro-apoptosis, pro-survival, and BH3-only. At a high level, the balance of binding of BH3-only proteins to the pro-apoptosis and pro-survival members of the BCL-2 family can determine whether a cell will undergo apoptosis. The protein BCL-X_{L}, encoded by the BCL2L1 gene, is a pro-survival member of the BCL-2 family. In many cancers, it can be desirable to initiate apoptosis of tumor cells, which may be achieved by decreasing the amount of pro-survival protein (e.g., BCL-X_{L}) available to compete for BH3-only protein binding.

### SUMMARY

This disclosure provides compounds of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) θ(e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (I-a)), or pharmaceutically acceptable salts thereof, that induce degradation of a BCL-X_{L} protein. These compounds are useful, for example, for treating a cancer in a subject (e.g., a human). This disclosure also provides compositions containing the compounds provided herein as well as methods of using and making the same.

Provided herein are compounds of Formula (I) or (II):

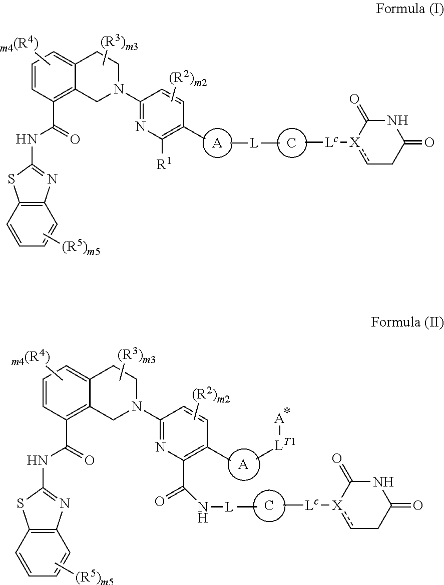

or pharmaceutically acceptable salts thereof, wherein:
Ring A is selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) phenylene or 5-6 membered heteroarylene, each of which is optionally substituted with 1-3 substituents independently selected from the group consisting of: R^{a} and R^{b},
L^{T1} is a bond or C₁₋₃ alkylene optionally substituted with 1-3 substituents independently selected from the group consisting of: oxo and R^{c}, wherein one CH₂ unit of the C₁₋₃ alkylene is optionally replaced with —O— or —N(R^{d})—;
A* is selected from the group consisting of:
(a) C₃₋₁₅ cycloalkyl or 3-15 membered heterocyclyl, each of which is optionally substituted with 1-6 substituents independently selected from the group consisting of: R^{a} and R^{b};
(b) C₆₋₁₅ aryl or 5-15 membered heteroaryl, each of which is optionally substituted with 1-6 substituents independently selected from the group consisting of: R^{a} and R^{b}; and
(c) H;
R¹ is selected from the group consisting of:
(a) C(O)OH,
(b) C(O)NR^{d}R^{e}, and
(c) C(O)OC₁₋₆ alkyl, wherein the C₁₋₆ alkyl is optionally substituted with 1-3 R^{c};
each R², R³, R⁴, and R⁵ is independently selected from the group consisting of: halo, CN, C₁₋₃ alkyl, C₁₋₃ haloalkyl, C₁₋₃ alkoxy, C₁₋₃ haloalkoxy, OH, and NR^{d}R^{e};
m2 is 0, 1, or 2;
m3 and m4 are independently 0, 1, 2, or 3;
m5 is 0, 1, 2, 3, or 4;
L is -(L^{A})ₙ₁-, wherein L^{A} and n1 are defined according to (AA) or (BB):
   (AA)
n1 is an integer from 3 to 15; and
each L^{A} is independently selected from the group consisting of: L^{A1} L^{A3}, and L^{A4}, provided that 1-3 occurrences of L^{A} is L^{A4};
   (BB)
n1 is an integer from 0 to 20; and
each L^{A} is independently selected from the group consisting of: L^{A1} and L^{A3};
each L^{A1} is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any N—O, O—O, N—N, N—S(O)₀, or O—S(O)₀₋₂ bonds;
wherein each R^{L} is independently selected from the group consisting of: halo; cyano; —OH; —C₁₋₆ alkoxy; —C₁₋₆ haloalkoxy; —NR^{d}R^{e}; C(═O)N(R^{f})₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{c};
Ring C is selected from the group consisting of:

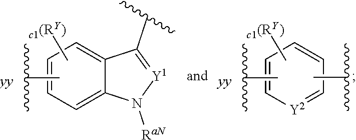

c1 is 0, 1, 2, or 3;
each R^{Y} is independently selected from the group consisting of R^{a} and R^{b};
R^{aN} is H or C₁₋₆ alkyl optionally substituted with 1-3 R^{c};
Y¹ and Y² are independently N, CH, or CR^{Y};
yy represents the point of attachment to L;
X is CH, C, or N;
the [symbol] is a single bond or a double bond;
L^{C} is selected from the group consisting of: a bond, —CH₂—, —CHR^{a}—, —C(R^{a})₂—, —N(R^{d})—, and O, provided that when X is N, then L^{C} is other than O;
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—; —N(H)—; —N(C₁₋₃ alkyl)-; —S(O)₀₋₂—; C(═O); and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(R^{f})₂; S(O)₁₋₂(C₁₋₆ alkyl); S(O)₁₋₂(C₁₋₆ haloalkyl); S(O)₁₋₂N(R^{f})₂; and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂.

Also provided herein is a pharmaceutical composition comprising a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, and a pharmaceutically acceptable carrier.

Provided herein is a method for treating cancer in a subject in need thereof, the method comprising administering to the subject a therapeutically effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, or a pharmaceutical composition as provided herein.

Also provided herein is a BCL-X_{L} protein non-covalently bound with a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof.

Also provided herein is a ternary complex comprising a BCL-X_{L} protein, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, and a CRBN protein, or a portion thereof.

To facilitate understanding of the disclosure set forth herein, a number of additional terms are provided. Generally, the nomenclature used herein and the laboratory procedures in organic chemistry, medicinal chemistry, and pharmacology described are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

The details of one or more embodiments of the invention are set forth in the accompanying drawings and the description below. Other features and advantages of the invention will be apparent from the description and drawings, and from the claims.

### DETAILED DESCRIPTION

This disclosure provides compounds of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or pharmaceutically acceptable salts thereof, that induce degradation of a BCL-X_{L} (also referred to as Bcl-xL herein) protein. These compounds are useful, for example, for treating a cancer. This disclosure also provides compositions containing the compounds provided herein as well as methods of using and making the same.

Without being bound by any particular theory, it is believed that in healthy cells, pro-apoptotic effectors such as BAX and BAK can move between the cytosol and the mitochondrial outer membrane (MOM), on which Voltage Dependent Anion Channel 2 (VDAC2) can act as a receptor. Pro-survival BCL-2 family members (e.g., BCL-2, BCL-X_{L}, and MCL-1) can retrotranslocate BAX back to the cytosol. A BH3-only protein (e.g., BIM) can engage a rear site on a pro-apoptotic effector (e.g., BAX or BAK) and release the C-terminal transmembrane domain (α9) of the effector, enabling binding to the MOM. Binding of BIM to the canonical BH3 binding groove of BAX or BAK releases the N-terminus and α1 of BAX or BAK, and subsequent unfolding of the “latch” domain releases BIM from the BH3 binding groove. If a pro-survival BCL-2 family member then binds to BAX or BAK, apoptotic signaling is generally halted. However, if BAX or BAK are allowed to dimerize, then oligomerize, the MOM can be permeabilized, leading to apoptosis.

An abundance of pro-survival BCL-2 family members is sometimes thought to “prime” cells for death (e.g., via cytotoxic therapies, including BH3 mimetics). It is believed that oncogenic mutations and stresses can cause an upregulation in BH3-only proteins, thus placing a selective pressure on cancer cells for upregulation of the pro-survival BCL-2 family proteins. Thus, with more BH3-only proteins around, the cells are believed to be more sensitive to further manipulations of the BCL-2 family balance. See, e.g., Adams and Cory, Cell Death & Differentiation 25.1 (2018): 27-36.

Compounds that induce degradation of a target protein are sometimes referred to as heterobifunctional compounds, PROTACs, or degraders. Such compounds generally include a moiety that binds to the target protein and a moiety that binds to a ubiquitin E3 ligase (sometimes referred to as an E3 ligase or simply an E3), these two moieties being optionally separated by a linker. To induce degradation, heterobifunctional compounds are believed to induce formation of a ternary complex between the target protein, the compound, and an E3 ligase. Formation of the ternary complex is then followed by ubiquitination of the target protein and degradation of the ubiquitinated target protein by a proteosome. Several E3 ligases have been used as the partner E3 ligase for heterobifunctional degraders. Herein, the cereblon (CRBN) E3 ligase (also referred to herein as a CRBN protein) is used.

A degradation approach for a target protein can have potential advantages compared to, for example, small molecule inhibition of the target protein. One potential advantage is that the duration of effect of a heterobifunctional compound is generally based on the resynthesis rate of the target protein. Another potential advantage is that many heterobifunctional compounds are believed to be released from the ubiquitinated target protein-E3 ligase complex and made available for formation of further ternary complexes; this is sometimes referred to as “catalytic” turnover of the heterobifunctional compound. Degradation of a target protein can also be advantageous over small molecule inhibition in some cases, as degradation can impair a scaffolding function of a target protein, whereas a small molecule might not. It is also generally believed that for formation of a ternary complex, high affinity to the target protein is not always required.

Heterobifunctional compounds are further described in, for example, International Publication Nos. WO 2017/184995; WO 2019/144117; WO 2020/163823; WO 2021/078301; WO 2021/146536; WO 2021/007307; WO 2021/222114; WO 2022/169780; WO 2023/044046; Chamberlain and Hamann, Nature Chemical Biology 15.10 (2019): 937-944; Li and Song, Journal of Hematology & Oncology 13 (2020): 1-14; Wu, et al. Nature Structural & Molecular Biology 27.7 (2020): 605-614; Dong, et al., Journal of Medicinal Chemistry 64.15 (2021): 10606-10620; Yang, et al., Targeted Oncology 16.1 (2021): 1-12; Lv, et al., Nature Communications 12.1 (2021): 6896.

### Compound Embodiments

Provided herein are compounds of Formula (I) or (II):

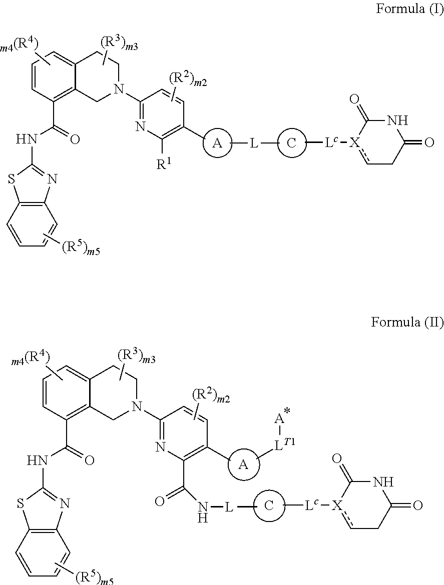

or pharmaceutically acceptable salts thereof, wherein:
Ring A is selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) phenylene or 5-6 membered heteroarylene, each of which is optionally substituted with 1-3 substituents independently selected from the group consisting of: R^{a} and R^{b},
L^{T1} is a bond or C₁₋₃ alkylene optionally substituted with 1-3 substituents independently selected from the group consisting of: oxo and R^{c}, wherein one CH₂ unit of the C₁₋₃ alkylene is optionally replaced with —O— or —N(R^{d})—;
A* is selected from the group consisting of:
(a) C₃₋₁₅ cycloalkyl or 3-15 membered heterocyclyl, each of which is optionally substituted with 1-6 substituents independently selected from the group consisting of: R^{a} and R^{b};
(b) C₆₋₁₅ aryl or 5-15 membered heteroaryl, each of which is optionally substituted with 1-6 substituents independently selected from the group consisting of: R^{a} and R^{b}; and
(c) H;
R¹ is selected from the group consisting of:
(a) C(O)OH,
(b) C(O)NR^{d}R^{e}, and
(c) C(O)OC₁₋₆ alkyl, wherein the C₁₋₆ alkyl is optionally substituted with 1-3 R^{c};
each R², R³, R⁴, and R⁵ is independently selected from the group consisting of: halo, CN, C₁₋₃ alkyl, C₁₋₃ haloalkyl, C₁₋₃ alkoxy, C₁₋₃ haloalkoxy, OH, and NR^{d}R^{e};
m2 is 0, 1, or 2;
m3 and m4 are independently 0, 1, 2, or 3;
m5 is 0, 1, 2, 3, or 4;
L is -(L^{A})ₙ₁-, wherein L^{A} and n1 are defined according to (AA) or (BB):
   (AA)
n1 is an integer from 3 to 15; and
each L^{A} is independently selected from the group consisting of: L^{A1} L^{A3}, and L^{A4}, provided that 1-3 occurrences of L^{A} is L^{A4};
   (BB)
n1 is an integer from 0 to 20; and
each L^{A} is independently selected from the group consisting of: L^{A1} and L^{A3};
each L^{A1} is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any N—O, O—O, N—N, N—S(O)₀, or O—S(O)₀₋₂ bonds;
wherein each R^{L} is independently selected from the group consisting of: halo; cyano; —OH; —C₁₋₆ alkoxy; —C₁₋₆ haloalkoxy; —NR^{d}R^{e}; C(═O)N(R^{f})₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{c};
Ring C is selected from the group consisting of:

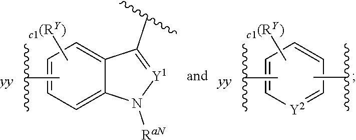

c1 is 0, 1, 2, or 3;
each R^{Y} is independently selected from the group consisting of: R^{a} and R^{b};
R^{aN} is H or C₁₋₆ alkyl optionally substituted with 1-3 R^{c};
Y¹ and Y² are independently N, CH, or CR^{Y};
yy represents the point of attachment to L;
X is CH, C, or N;
the [symbol] is a single bond or a double bond;
L^{C} is selected from the group consisting of: a bond, —CH₂—, —CHR^{a}—, —C(R^{a})₂—, —N(R^{d})—, and O, provided that when X is N, then L^{C} is other than O;
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{e};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—; —N(H)—; —N(C₁₋₃ alkyl)-; —S(O)₀₋₂—; C(═O); and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(Rr)₂; S(O)₁₋₂(C₁₋₆ alkyl); S(O)₁₋₂(C₁₋₆ haloalkyl); S(O)₁₋₂N(R^{f})₂; and C₁₋₆ alkyl optionally substituted with 1-3 R^{h}.
each R^{f} is independently selected from the group consisting of: H and C₁₋₆ alkyl optionally substituted with 1-3 R^{h};
each R^{g} is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂.

In some embodiments, the compounds are compounds of Formula (I), or pharmaceutically acceptable salts thereof.

In some embodiments, the compounds are compounds of Formula (II), or pharmaceutically acceptable salts thereof.

In some embodiments, Ring A is phenylene or 5-6 membered heteroarylene, each of which is optionally substituted with 1-3 substituents independently selected from the group consisting of: R^{a} and R^{b}.

In some embodiments, Ring A is phenylene optionally substituted with 1-3 substituents independently selected from the group consisting of: R^{a} and R^{b}. In some embodiments, Ring A is a phenylene optionally substituted with 1-3 R^{a}.

In some embodiments, Ring A is phenylene (e.g., 1,3-phenylene or 1,4-phenylene).

In some embodiments, Ring A is

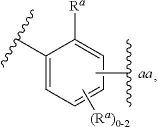

wherein aa represents the point of attachment to L or L^{T1}.

In some embodiments, Ring A is

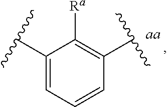

wherein aa represents the point of attachment to L or L^{T1}. For example, Ring A can be

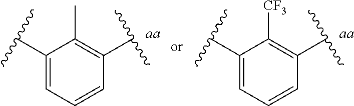

wherein aa represents the point of attachment to L or L^{T1}.

In some embodiments, Ring A is

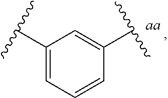

wherein aa represents the point of attachment to L or L^{T1}.

In some embodiments, Ring A is

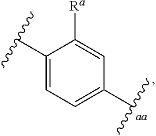

wherein aa represents the point of attachment to L or L^{T1}. For example, Ring A can be

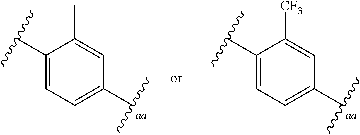

wherein aa represents the point of attachment to L or L^{T1}.

In some embodiments, Ring A is 5-6 membered heteroarylene optionally substituted with 1-3 R^{a}. In some embodiments, Ring A is 5-6 membered heteroarylene optionally substituted with 1-2 R^{a}. In some embodiments, Ring A is 5-membered heteroarylene optionally substituted with 1-2 R^{a}. In some embodiments, Ring A is pyrazolylene optionally substituted with 1-2 R^{a}. For example, Ring A can be selected from the group consisting of:

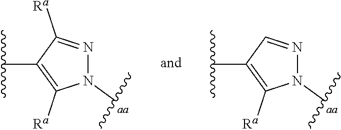

wherein aa represents the point of attachment to L or L^{T1}.

In some embodiments, Ring A is C₃₋₁₀ cycloalkylene optionally substituted with 1-6 R^{a}. In some embodiments, Ring A is C₄₋₆ cycloalkylene optionally substituted with 1-3 R^{a}. In some embodiments, Ring A is cyclohexylene optionally substituted with 1-3 R^{a}. For example, Ring A can be 1,4-cyclohexylene.

In some embodiments, one R^{a} present on Ring A is C₁₋₃ alkyl optionally substituted with 1-3 F. In some embodiments, one R^{a} present on Ring A is methyl or CF₃.

In some embodiments, R¹ is C(O)OH. In some embodiments, R¹ is C(O)OC₁₋₆ alkyl. In some embodiments, R¹ is C(O)NHR^{e}.

In some embodiments, R¹ is C(O)OH; and Ring A is

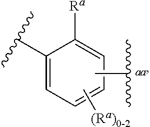

wherein aa represents the point of attachment to L or L^{T1}. In some embodiments, Ring A is

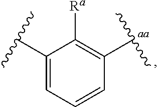

wherein aa represents the point of attachment to L or L^{T1}. For example, Ring A can be

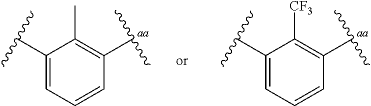

wherein aa represents the point of attachment to L or L^{T1}. In some embodiments, Ring A is

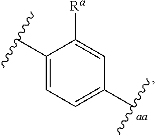

wherein aa represents the point of attachment to L or L^{T1}. For example, Ring A can be

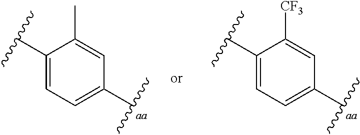

wherein aa represents the point of attachment to L or L^{T1}.

In some embodiments, R¹ is C(O)OH; and Ring A is

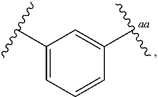

wherein aa represents the point of attachment to L or L^{T1}.

In some embodiments, R¹ is C(O)OH or C(O)OC₁₋₆ alkyl; and Ring A is selected from the group consisting of:

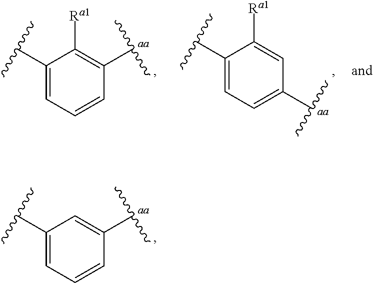

wherein R^{a1} is C₁₋₃ alkyl optionally substituted with 1-3 F; and aa represents the point of attachment to L or L^{T1}.

In some embodiments, L^{T1} is C₁₋₃ alkylene. For example, L^{T1} can be —CH₂—.

In some embodiments, A* is H. In some embodiments, A* is C₃₋₁₅ cycloalkyl or 3-15 membered heterocyclyl, each of which is optionally substituted with 1-6 substituents independently selected from the group consisting of: R^{a} and R^{b}. In some embodiments, A* is C₃₋₁₅ cycloalkyl optionally substituted with 1-3 R^{a}. For example, A* can be adamantyl optionally substituted with 1-3 R^{a}.

In some embodiments, m2 is 0. In some embodiments, m3 is 0. In some embodiments, m4 is 0. In some embodiments, m5 is 0.

In some embodiments, m2 is 0; m3 is 0; m4 is 0; and m5 is 0.

In some embodiments, m2 is 0; m3 is 0; m4 is 0; and m5 is 0;
R¹ is C(O)OH or C(O)OC₁₋₆ alkyl; and
Ring A is selected from the group consisting of:

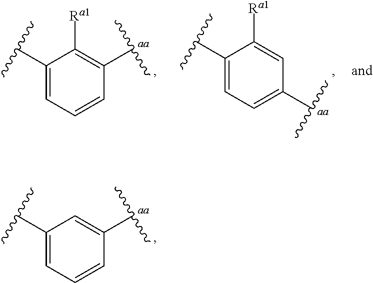

wherein R^{a1} is C₁₋₃ alkyl optionally substituted with 1-3 F; and aa represents the point of attachment to L or L^{T1}.

In some embodiments, m2 is 0; m3 is 0; m4 is 0; and m5 is 0;
R¹ is C(O)OH or C(O)OC₁₋₆ alkyl; and
Ring A is selected from the group consisting of:

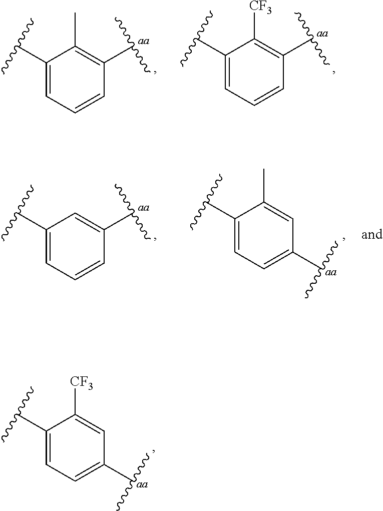

wherein aa represents the point of attachment to L or L^{T1}.

In some embodiments, Ring C is

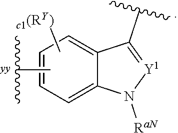

In some embodiments, Ring C is

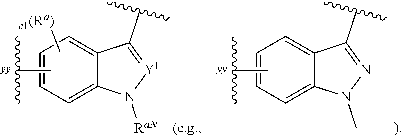

For example, Ring C can be

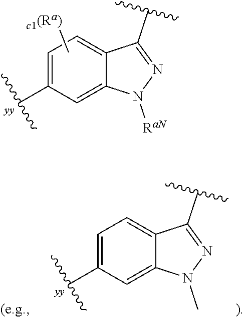

For example, Ring C can be

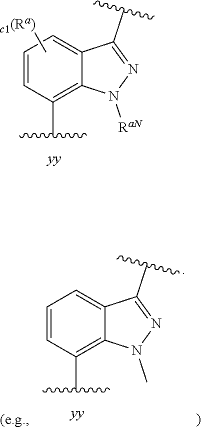

For example, Ring C can be

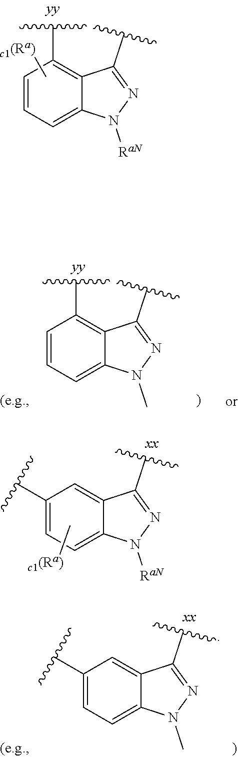

In some embodiments, Ring C is

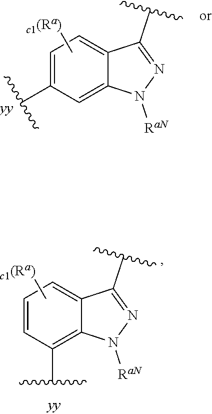

wherein c1 is 0 or 1; and R^{a2} is selected from the group consisting of: halo (e.g., —F) and C₁₋₃ alkyl optionally substituted with 1-3-F. In some embodiments, Ring C is

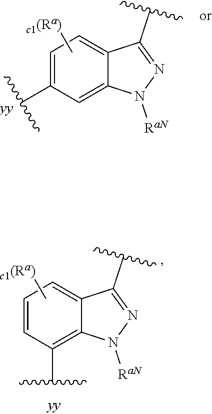

wherein R^{aN} is C₁₋₃ alkyl (e.g., methyl); c1 is 0 or 1; and R^{a2} is selected from the group consisting of: halo (e.g., —F) and C₁₋₃ alkyl optionally substituted with 1-3-F. In some embodiments, Ring C is selected from the group consisting of:

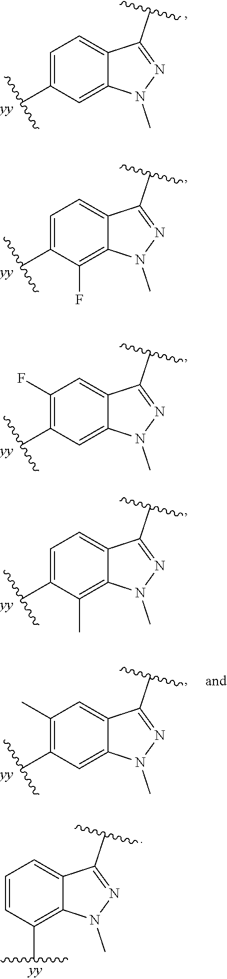

In some embodiments, Ring C is

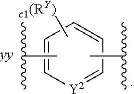

In some embodiments, Ring C is

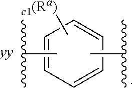

For example, Ring C can be

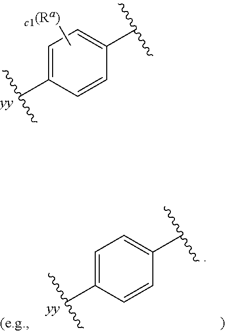

For example, Ring C can be

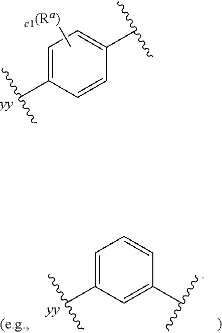

In some embodiments, c1 is 0.

In some embodiments, c1 is 1. In some embodiments, c1 is 1; and R^{Y} is R^{a}. In some embodiments, R^{Y} is —F, C₁₋₃ alkyl, or C₁₋₃ alkyl substituted with 1-3-F.

In some embodiments, R^{aN} is C₁₋₃ alkyl. For example, R^{aN} can be methyl.

In some embodiments, L^{C} is a bond or —N(R^{d})—. In some embodiments, L^{C} is a bond or —N(H)—. For example, L^{C} can be a bond. For example, L^{C} can be —NH—.

In some embodiments, X is CH.

In some embodiments, the

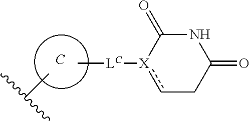

moiety is

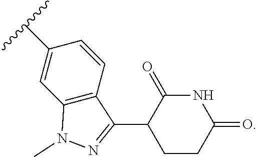 [SMILES: *c1ccc2c(C3CCC(=O)NC3=O)nn(C)c2c1]

In some embodiments, the

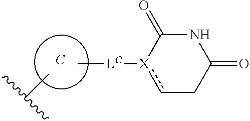

moiety is

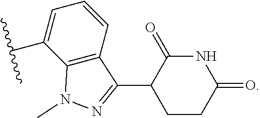 [SMILES: *c1cccc2c(C3CCC(=O)NC3=O)nn(C)c12]

In some embodiments, the

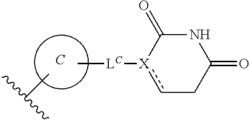

moiety is

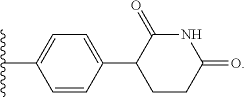 [SMILES: *c1ccc(C2CCC(=O)NC2=O)cc1]

In some embodiments, the

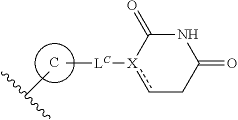

moiety is

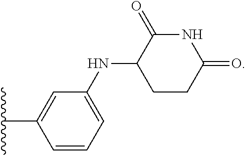 [SMILES: *c1cccc(NC2CCC(=O)NC2=O)c1]

In some embodiments, L is -(L^{A})ₙ₁-; and L^{A} and n1 are defined according to (AA). In some embodiments, n1 is an integer from 3 to 5. In some embodiments, n1 is an integer from 5 to 9. In some embodiments, n1 is 6, 7, or 8. In some embodiments, n1 is an integer from 9 to 12.

In some embodiments, L^{A} and n1 are defined according to (AA); and 1-2 occurrences of L^{A} is L^{A4}. In some embodiments, one occurrence of L^{A} is L^{A4}. In some embodiments, two occurrences of L^{A} are L^{A4}. In some embodiments, each L^{A4} is independently selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

In some embodiments, L^{A} and n1 are defined according to (AA); and 1-4 occurrences of L^{A} is L^{A3}. In some embodiments, 1-3 (e.g., 1-2 or 2-3) occurrences of L^{A} is L^{A3}. In some embodiments, 0-1 occurrence of L^{A3} is C(═O); and each remaining occurrence of L^{A3} is independently selected from the group consisting of: —O—; —N(H)—; and —N(C₁₋₃ alkyl)-.

In some embodiments, L^{A} and n1 are defined according to (AA); and 2-7 occurrences of L^{A} are L^{A1}. In some embodiments, 2-5 occurrences of L^{A} are L^{A1}. In some embodiments, 0-2 (e.g., 0-1) occurrences of L^{A1} are —CHR^{L}— or —C(R^{L})₂—; and each remaining occurrence of L^{A1} is —CH₂—. In some embodiments, each occurrence of L^{A1} is —CH₂—. In some embodiments, one occurrence of L^{A1} is —CHR^{L}— or —C(R^{L})₂—; and each remaining occurrence of L^{A1} is —CH₂—. In some embodiments, each R^{L} is independently selected from the group consisting of: —F and —C₁₋₃ alkyl optionally substituted with 1-3 F (e.g., CH₃ or CF₃).

In some embodiments, L is -(L^{A})ₙ₁-, and L^{A} and n1 are defined according to (AA), wherein n1 is an integer from 5 to 9; 1-2 occurrences of L^{A} is L^{A4}; 2-7 occurrences of L^{A} are L^{A1}; and 1-3 occurrences of L^{A} is L^{A3}. In some embodiments, each L^{A4} is independently selected from the group consisting of: C₃₋₁₀ cycloalkylene or 4-10 membered heterocyclylene, each of which is optionally substituted with 1-3 R^{a}; and phenylene or 5-6 membered heteroarylene, each of which is optionally substituted with 1-3 R^{a}. In some embodiments, 0-1 occurrence of L^{A1} is —CHR^{L}— or —C(R^{L})₂—; and each remaining occurrence of L^{A1} is —CH₂—. In some embodiments, 0-1 occurrence of L^{A3} is C(═O); and each remaining occurrence of L^{A3} is independently selected from the group consisting of: —O—; —N(H)—; and —N(C₁₋₃ alkyl)-.

In some embodiments, L is selected from the group consisting of:
(i) -(L^{A3})₀₋₂-(L^{A1})₀₋₅-L^{A4}-(L^{A1})₀₋₅-(L^{A3})₁₋₂-_{bb};
(ii) -(L^{A3})₀₋₂-(L^{A1})₀₋₅-L^{A4}-(L^{A1})₀₋₅-L^{A4}-_{bb};
(iii) -(L^{A3})₀₋₂-(L^{A1})₀₋₅-L^{A4}-(L^{A1})₀₋₅-L^{A3}-L^{A4}-_{bb}; and
(iv) -(L^{A3})₀₋₁-(L^{A1})₂₋₃-L^{A3}-L^{A4}-(L^{A1})₀₋₅-(L^{A3})₁₋₂-bb;
   provided that L contains 2-7 L^{A1}; and wherein bb represents the point of attachment to Ring C. In some embodiments, each L^{A4} is independently selected from the group consisting of: C₃₋₁₀ cycloalkylene or 4-10 membered heterocyclylene, each of which is optionally substituted with 1-3 R^{a}; and phenylene or 5-6 membered heteroarylene, each of which is optionally substituted with 1-3 R^{a}. In some embodiments, 0-1 occurrence of L^{A1} is —CHR^{L}— or —C(R^{L})₂—; and each remaining occurrence of L^{A1} is —CH₂—. In some embodiments, 0-1 occurrence of L^{A3} is C(═O); and each remaining occurrence of L^{A3} is independently selected from the group consisting of: —O—; —N(H)—; and —N(C₁₋₃ alkyl)-.

In some embodiments, L is -(L^{A3})₀₋₂-(L^{A1})₂₋₉-L^{A3})₀₋₁-L^{A4}-_{bb}, wherein bb represents the point of attachment to Ring C. In some embodiments, L^{A4} is selected from the group consisting of: C₃₋₁₀ cycloalkylene or 4-10 membered heterocyclylene, each of which is optionally substituted with 1-3 R^{a}; and phenylene or 5-6 membered heteroarylene, each of which is optionally substituted with 1-3 R^{a}. In some embodiments, L^{A4} is 4-10 membered heterocyclylene (e.g., 5-6 membered heterocyclylene (e.g., 6-membered heterocyclylene (e.g., piperazinylene))) optionally substituted with 1-3 R^{a}. In some embodiments, 0-1 occurrence of L^{A1} is —CHR^{L}— or —C(R^{L})₂—; and each remaining occurrence of L^{A1} is —CH₂—. In some embodiments, 0-1 occurrence of L^{A3} is C(═O); and each remaining occurrence of L^{A3} is independently selected from the group consisting of: —O—; —N(H)—; and —N(C₁₋₃ alkyl)-.

In some embodiments, L is: -(L^{A3})₀₋₂-(L^{A1})₀₋₅-L^{A4}-(L^{A1})₀₋₅-(L^{A3})₁₋₂-_{bb}; provided that L contains 1-7 (e.g., 2-7) L^{A1}; and wherein bb represents the point of attachment to Ring C. In some embodiments, L is: -(L^{A3})₀₋₁-(L^{A1})₀₋₅-L^{A4}-(L^{A1})₀₋₅-(L^{A3})₂-_{bb}; provided that L contains 1-5 (e.g., 2-5) L^{A1}; and wherein bb represents the point of attachment to Ring C. In some embodiments, L^{A4} is selected from the group consisting of: C₃₋₁₀ cycloalkylene or 4-10 membered heterocyclylene, each of which is optionally substituted with 1-3 R^{a}; and phenylene or 5-6 membered heteroarylene, each of which is optionally substituted with 1-3 R^{a}. In some embodiments, 0-1 occurrence of L^{A1} is —CHR^{L}— or —C(R^{L})₂—; and each remaining occurrence of L^{A1} is —CH₂—. In some embodiments, 0-1 occurrence of L^{A3} is C(═O); and each remaining occurrence of L^{A3} is independently selected from the group consisting of: —O—; —N(H)—; and —N(C₁₋₃ alkyl)-.

In some embodiments, L is: -(L^{A3})₀₋₁-(L^{A1})₀₋₅-L^{A4}-(L^{A1})₀₋₅-C(═O)—N(R^{d})-_{bb}; provided that L contains 1-5 (e.g., 2-5) L^{A1}; and wherein bb represents the point of attachment to Ring C. In some embodiments, L^{A4} is selected from the group consisting of: C₃₋₁₀ cycloalkylene or 4-10 membered heterocyclylene, each of which is optionally substituted with 1-3 R^{a}; and phenylene or 5-6 membered heteroarylene, each of which is optionally substituted with 1-3 R^{a}. For example, L^{A4} can be a 4-10 membered (e.g., nitrogen containing) heterocyclylene optionally substituted with 1-3 R^{a}. In some embodiments, 0-1 occurrence of L^{A1} is —CHR^{L}— or —C(R^{L})₂—; and each remaining occurrence of L^{A1} is —CH₂—. For example, L can be selected from the group consisting of:

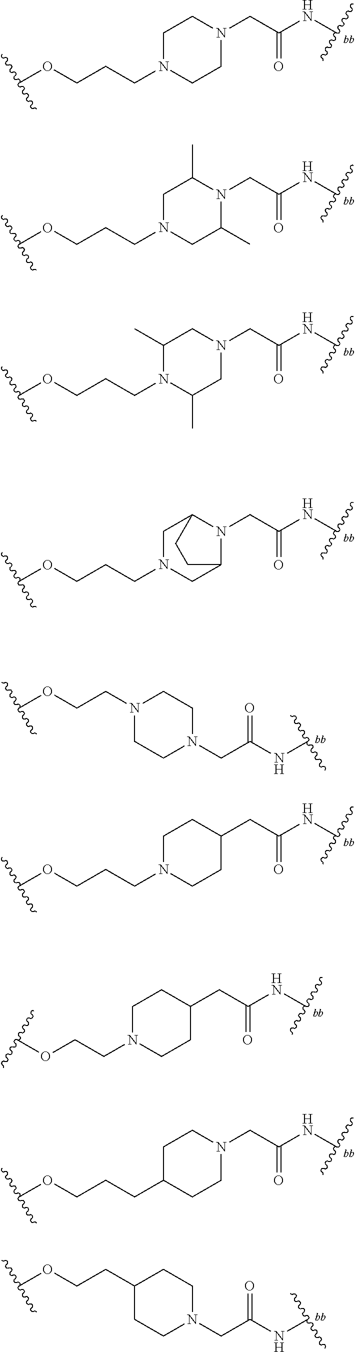

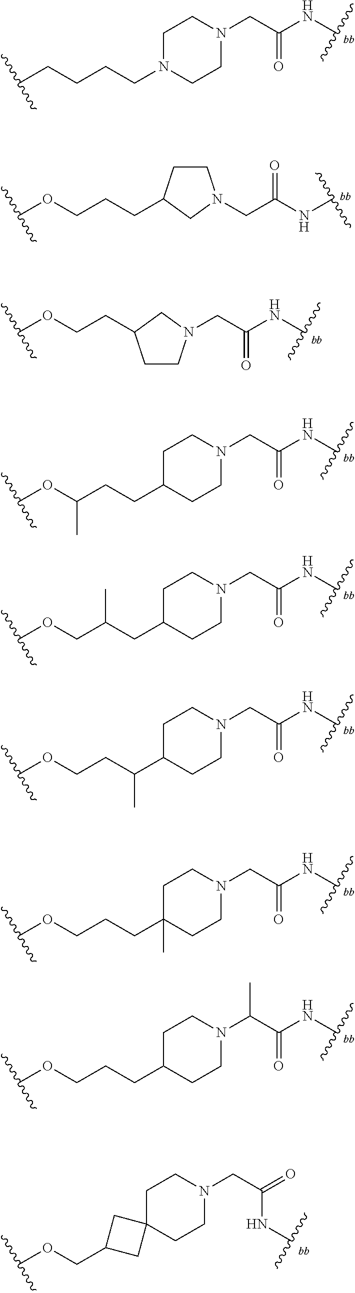

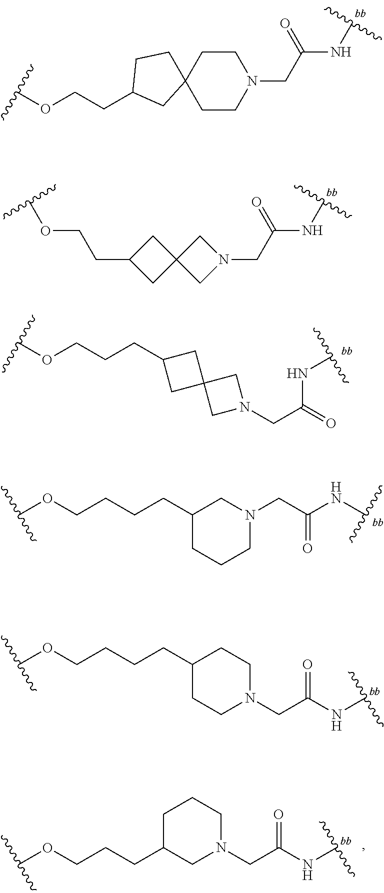

wherein bb represents the point of attachment to Ring C. For example, L can be selected from the group consisting of:

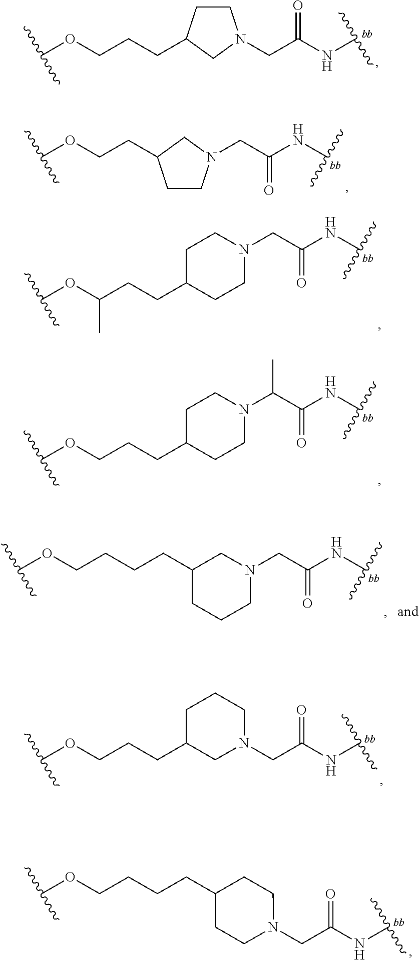

wherein bb represents the point of attachment to Ring C. For example, L can be selected from the group consisting of:

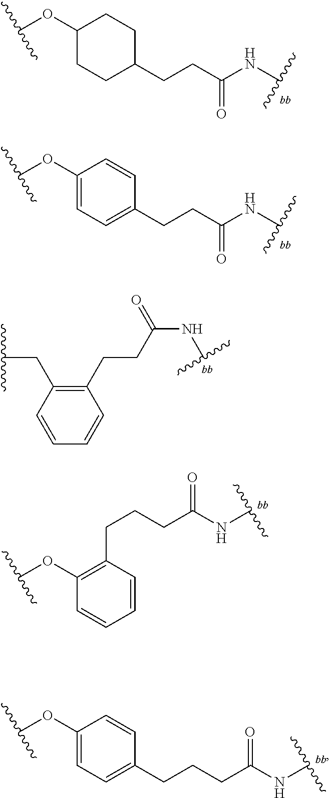

wherein bb represents the point of attachment to Ring C.

In some embodiments, L is a divalent group of Formula (L-1):

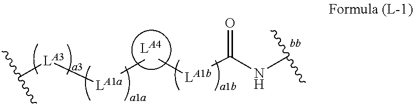

wherein:
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 1 to 5 (e.g., from 2 to 5);
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
bb represents the point of attachment to Ring C.

In some embodiments of (L-1), a3 is 1. In some embodiments, L^{A3} is —O—. In some embodiments of (L-1), a3 is 1; and L^{A3} is —O—. In some embodiments of (L-1), a3 is 0.

In some embodiments of (L-1), a1a+a1b is 3 or 4. For example, a1a+a1b can be 3. For example, a1a+a1b can be 4.

In some embodiments of (L-1), a1a+a1b is 2 or 5. For example, a1a+a1b can be 5. For example, a1a+a1b can be 2.

In some embodiments of (L-1), a1a+a1b is 1.

In some embodiments of (L-1), each occurrence of L^{A1a} and L^{A1b} is —CH₂—. In some embodiments of (L-1), one occurrence of L^{A1a} is —CHR^{L}— or —C(R^{L})₂—; each remaining occurrence of L^{A1a} is —CH₂—; and each occurrence of L^{A1b} is —CH₂—. In some embodiments of (L-1), one occurrence of L^{A1b} is —CHR^{L}— or —C(R^{L})₂—; each remaining occurrence of L^{A1b} is —CH₂—; and each occurrence of L^{A1a} is —CH₂—.

In some embodiments, L is: -(L^{A3})₀₋₁-(L^{A1})₀₋₅-L^{A4}-(L^{A1})₀₋₅-L^{A3}-bb; provided that L contains 2-7 L^{A1}; and wherein bb represents the point of attachment to Ring C. In some embodiments, L is: -(L^{A3})₀₋₁-(L^{A1})₀₋₅-L^{A4}-(L^{A1})₀₋₅-O-b, -(L^{A3})₀₋₁-(L^{A1})₀₋₅-L^{A4}-(L^{A1})₀₋₅-N(H)—_{bb}, or -(L^{A3})₀₋₁-(L^{A1})₀₋₅-L^{A4}-(L^{A1})₀₋₅-N(C₁₋₃ alkyl)-_{bb}; provided that L contains 2-7 L^{A1}; and wherein bb represents the point of attachment to Ring C. In some embodiments, L^{A4} is selected from the group consisting of: C₃₋₁₀ cycloalkylene or 4-10 membered heterocyclylene, each of which is optionally substituted with 1-3 R^{a}; and phenylene or 5-6 membered heteroarylene, each of which is optionally substituted with 1-3 R^{a}. In some embodiments, 0-1 occurrence of L^{A1} is —CHR^{L}— or —C(R^{L})₂—; and each remaining occurrence of L^{A1} is —CH₂—. For example, L can be

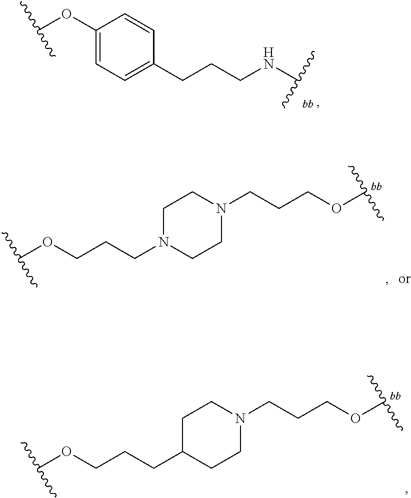

wherein bb represents the point of attachment to Ring C.

In some embodiments, L is: -(L^{A3})₀₋₁-(L^{A1})₀₋₅-L^{A4}-(L^{A1})₀₋₅-_{bb}; provided that L contains 2-7 L^{A1}; and wherein bb represents the point of attachment to Ring C. In some embodiments, L^{A4} is selected from the group consisting of: (a) C₃₋₁₀ cycloalkylene or 4-10 membered heterocyclylene, each of which is optionally substituted with 1-3 R^{a}; and (b) phenylene or 5-6 membered heteroarylene, each of which is optionally substituted with 1-3 R^{a}. In some embodiments, 0-1 occurrence of L^{A1} is —CHR^{L}— or —C(R^{L})₂—; and each remaining occurrence of L^{A1} is —CH₂—. For example, L can be

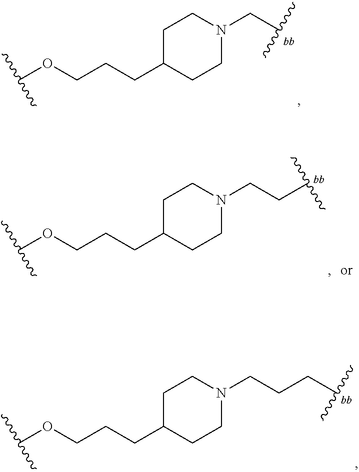

wherein bb represents the point of attachment to Ring C.

In some embodiments, L is a divalent group of Formula (L-2) or (L-2a):

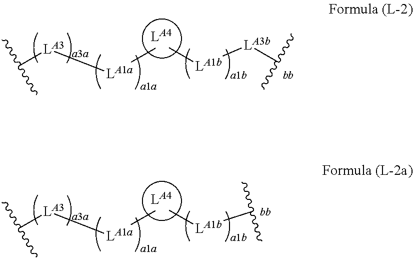

wherein:
a3a is 0 or 1;
L^{A3a} and L^{A3b} are independently selected from the group consisting of: —O—, —N(H)—, and —N(C₁₋₃ alkyl)-;
a1a and a1b are independently integers from 0 to 5, provided that a1a+a1b is from 2 to 7;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
bb represents the point of attachment to Ring C.

In some embodiments of (L-2) or (L-2a), a3a is 1. In some embodiments, L^{A3a} is —O—. In some embodiments of (L-2) or (L-2a), a3a is 1 and L^{A3a} is —O—.

In some embodiments of (L-2), L^{A3b} is —N(H)— or —N(C₁₋₃ alkyl)-. In some embodiments of (L-2), L^{A3}b is —O—.

In some embodiments of (L-2) or (L-2a), a1a+a1b is 2, 3, or 4. For example, a1a+a1b can be 3. For example, a1a+a1b can be 4. In some embodiments, a1a is 3.

In some embodiments of (L-2) or (L-2a), a1a+a1b is 5 or 6.

In some embodiments of (L-2) or (L-2a), each occurrence of L^{A1a} and L^{A1b} is —CH₂—. In some embodiments of (L-2) or (L-2a), one occurrence of L^{A1a} is —CHR^{L}— or —C(R^{L})₂—; each remaining occurrence of L^{A1a} is —CH₂—; and each occurrence of L^{A1b} is —CH₂—. In some embodiments of (L-2) or (L-2a), one occurrence of L^{A1b} is —CHR^{L}— or —C(R^{L})₂—; each remaining occurrence of L^{A1b} is —CH₂—; and each occurrence of L^{A1a} is —CH₂—.

In some embodiments, L is: -(L^{A3})₀₋₁-(L^{A1})₀₋₅-L^{A4}-(L^{A1})₀₋₅-L^{A4}-_{bb}; -(L^{A3})₀₋₁-(L^{A1})₀₋₅-L^{A4} (L^{A1})₀₋₅-L^{A3}-L^{A4}-bb; or -(L^{A3})₀₋₁-(L^{A1})₀₋₅-L^{A4}-(L^{A1})₀₋₅-L^{A4}-L^{A3}-_{bb}, provided that L contains 2-7 L^{A1}; and wherein bb represents the point of attachment to Ring C. In some embodiments, L is: -(L^{A3})₀₋₁-(L^{A1})₀₋₅-L^{A4}-(L^{A1})₀₋₅-(4-10 (e.g., 6) membered heterocyclylene)-_{bb}; -(L^{A3})₀₋₁-(L^{A1})₀₋₅-L^{A4}-(L^{A1})₀₋₅-L^{A3}-(4-10 (e.g., 6) membered heterocyclylene)-_{bb}; or -(L^{A3})₀₋₁-(L^{A1})₀₋₅-L^{A4}-(L^{A1})₀₋₅-L^{A3}-(4-10 (e.g., 6) membered heterocyclylene)-L^{A3}-_{bb}, provided that L contains 2-7 L^{A1}; wherein the 4-10 (e.g., 6) membered heterocyclylene is optionally substituted with 1-3 R^{a}; and wherein bb represents the point of attachment to Ring C.

In some embodiments, L is: -(L^{A3})₀₋₁-(L^{A1})₀₋₅-L^{A4}-(L^{A1})₀₋₅-(piperazinylene)-_{bb}; -(L^{A3})₀₋₁-(L^{A1})₀₋₅-L^{A4}(L^{A1})₀₋₅-(piperidinylene)-_{bb}; -(L^{A3})₀₋₁(L^{A1})₀₋₅-L^{A4}-(L^{A1})₀₋₅-(pyrrolidinylene)-_{bb}; -(L^{A3})₀₋₁-(L^{A1})₀₋₅-L^{A4}-(L^{A1})₀₋₅-C(═O)-(piperazinylene)-_{bb}; -(L^{A3})₀₋₁-(L^{A1})₀₋₅-L^{A4}-(L^{A1})₀₋₅-C(═O)-(piperidinylene)-_{bb}; or -(L^{A3})₀₋₁-(L^{A1})₀₋₅-L^{A4}-(L^{A1})₀₋₅-(piperidinylene)-O-_{bb}, provided that L contains 2-5 L^{A1}; wherein the piperazinylene, piperidinylene, and pyrrolidinylene are each optionally substituted with 1-3 R^{a}, and wherein bb represents the point of attachment to Ring C.

In some embodiments, 0-1 occurrence of L^{A1} is —CHR^{L}— or —C(R^{L})₂—; and each remaining occurrence of L^{A1} is —CH₂—.

In some embodiments, L is: -(L^{A3})₀₋₁-(L^{A1})₀₋₅-L^{A4}-(L^{A1})₀₋₅-(4-10 membered heterocyclylene)-_{bb}; or -(L^{A3})₀₋₁-(L^{A1})₀₋₅-L^{A4}-(L^{A1})₀₋₅-L^{A3}-(4-10 membered heterocyclylene)-_{bb}; provided that L contains 2-7 L^{A1}, further provided that the 4-10 membered heterocyclylene contains two ring nitrogen atoms and no additional ring heteroatoms, wherein the 4-10 membered heterocyclylene is optionally substituted with 1-3 R^{a}; and wherein bb represents the point of attachment to Ring C. In some embodiments, L is: -(L^{A3})₀₋₁-(L^{A1})₀₋₅-L^{A4}-(L^{A1})₀₋₅-(piperazinylene)-_{bb}; or -(L^{A3})₀₋₁-(L^{A1})₀₋₅-L^{A4}-(L^{A1})₀₋₅-C(═O)-(piperazinylene)-_{bb}; provided that L contains 2-5 L^{A1}; wherein the piperazinylene is optionally substituted with 1-3 R^{a}; and bb represents the point of attachment to Ring C. In some embodiments, L is: -(L^{A3})₀₋₁-(L^{A1})₀₋₅-L^{A4}-(L^{A1})₀₋₅-(piperazinylene)-_{bb}; or -(L^{A3})₀₋₁-(L^{A1})₀₋₅-L^{A4}-(L^{A1})₀₋₅-C(═O)-(piperazinylene)-_{bb}; provided that L contains 2-5 L^{A1}; and wherein bb represents the point of attachment to Ring C. In some embodiments, 0-1 occurrence of L^{A1} is —CHR^{L}— or —C(R^{L})₂—; and each remaining occurrence of L^{A1} is —CH₂—.

For example, L can be selected from the group consisting of:

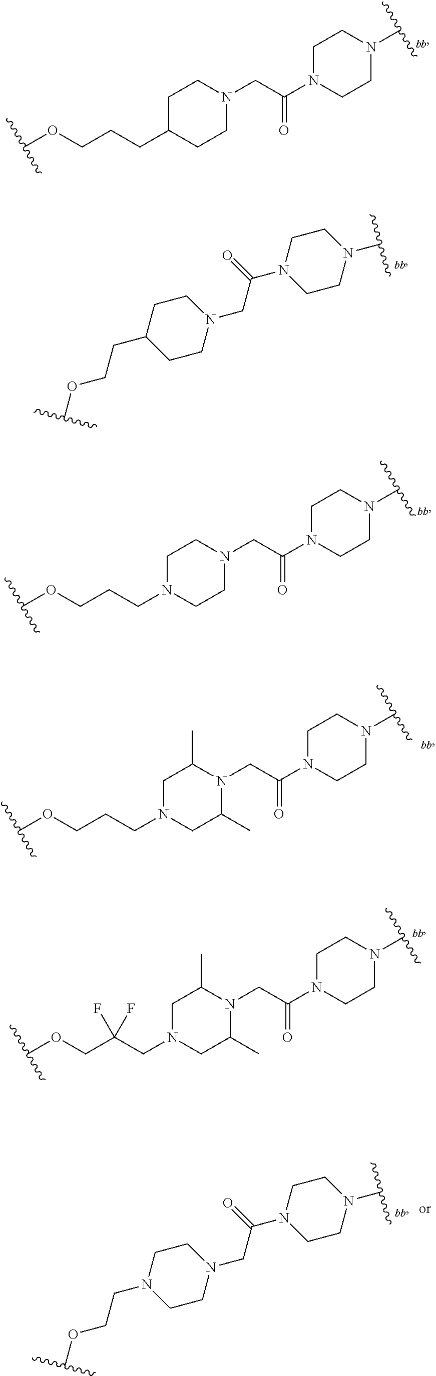

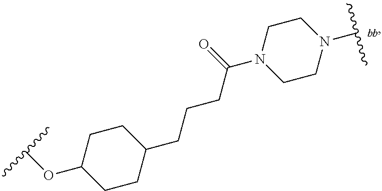

wherein bb represents the point of attachment to Ring C.

For example, L can be selected from the group consisting of:

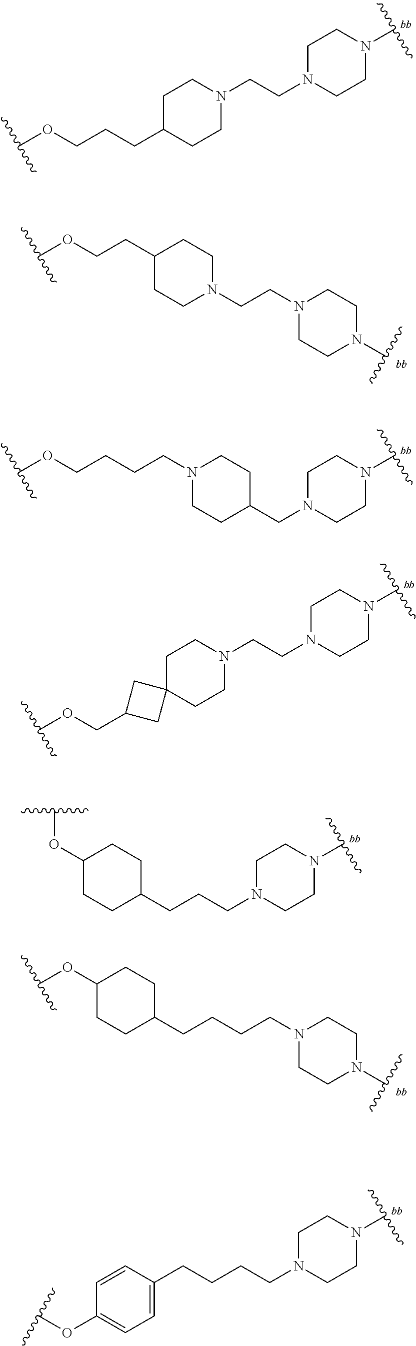

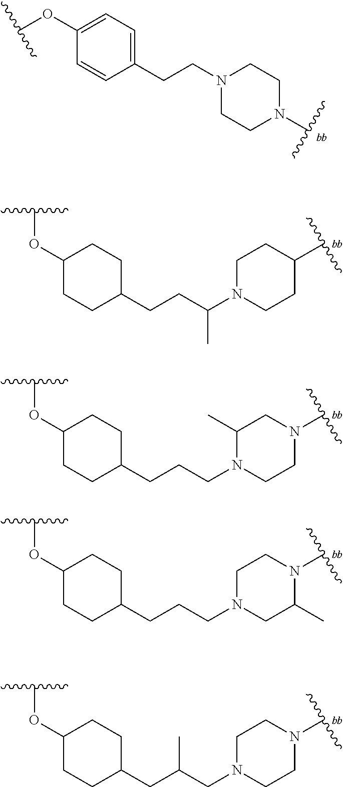

wherein bb represents the point of attachment to Ring C.

In some embodiments, L is a divalent group of Formula (L-3) or (L-4):

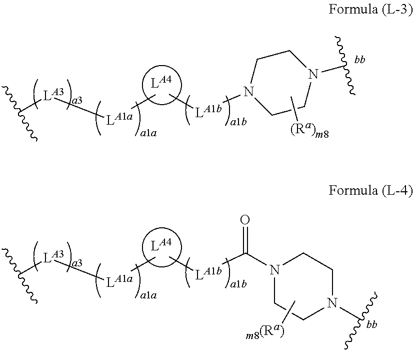

wherein:
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 1 to 5 (e.g., from 2 to 5);
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
m8 is 0, 1, or 2; and
bb represents the point of attachment to Ring C.

In some embodiments of (L-3) or (L-4), m8 is 0. In some embodiments of (L-3) or (L-4), m8 is 1. In some embodiments of (L-3) or (L-4), m8 is 2.

In some embodiments of (L-3) or (L-4), a3 is 1. In some embodiments, L^{A3} is 0. In some embodiments of (L-3) or (L-4), a3 is 1; and L^{A3} is —O—.

In some embodiments of (L-3) or (L-4), a1a+a1b is 3 or 4. For example, a1a+a1b can be 3. For example, a1a+a1b can be 4.

In some embodiments of (L-3) or (L-4), a1a+a1b is 5.

In some embodiments of (L-3) or (L-4), a1a+a1b is 2.

In some embodiments of (L-3) or (L-4), a1a+a1b is 2 to 5.

In some embodiments of (L-3) or (L-4), each occurrence of L^{A1a} and L^{A1b} is —CH₂—. In some embodiments of (L-3) or (L-4), one occurrence of L^{A1a} is —CHR^{L}— or —C(R^{L})₂—; each remaining occurrence of L^{A1a} is —CH₂—; and each occurrence of L^{A1b} is —CH₂—. In some embodiments of (L-3) or (L-4), one occurrence of L^{A1b} is —CHR^{L}— or —C(R^{L})₂—; each remaining occurrence of L^{A1b} is —CH₂—; and each occurrence of L^{A1a} is —CH₂—.

In some embodiments, L is: -(L^{A3})₀₋₁-(L^{A1})₀₋₅-L^{A4}-(L^{A1})₀₋₅-(4-10 (e.g., 6) membered heterocyclylene)-_{bb}; -(L^{A3})₀₋₁-(L^{A1})₀₋₅-L^{A4}-(L^{A1})₀₋₅-L^{A3}-(4-10 (e.g., 6) membered heterocyclylene)-_{bb}; or -(L^{A3})₀₋₁-(L^{A1})₀₋₅-L^{A4}-(L^{A1})₀₋₅-(4-10 (e.g., 6) membered heterocyclylene)-L^{A3}-_{bb}; provided that L contains 2-7 L^{A1}, further provided that the 4-10 (e.g., 6) membered heterocyclylene contains one ring nitrogen atom and no additional ring heteroatoms, wherein the 4-10 membered heterocyclylene is optionally substituted with 1-3 R^{a}; and wherein bb represents the point of attachment to Ring C. In some embodiments, L is: -(L^{A3})₀₋₁-(L^{A1})₀₋₅-L^{A4}-(L^{A1})₀₋₅-(piperidinylene)-_{bb}; -(L^{A3})₀₋₁-(L^{A1})₀₋₅-L^{A4}-(L^{A1})₀₋₅-C(═O)-(piperidinylene)-_{bb}; or -(L^{A3})₀₋₁-(L^{A1})₀₋₅-L^{A4}-(L^{A1})₀₋₅-(piperidinylene)-O-_{bb}; provided that L contains 2-5 L^{A1}; wherein the piperidinylene is optionally substituted with 1-3 R^{a}; and bb represents the point of attachment to Ring C. In some embodiments, L is: -(L^{A3})₀₋₁-(L^{A1})₀₋₅-L^{A4}-(L^{A1})₀₋₅-(piperidinylene)-_{bb}; provided that L contains 2-5 L^{A1}; and wherein bb represents the point of attachment to Ring C. In some embodiments, 0-1 occurrence of L^{A1} is —CHR^{L}— or —C(R^{L})₂—; and each remaining occurrence of L^{A1} is —CH₂—.

For example, L can be selected from the group consisting of:

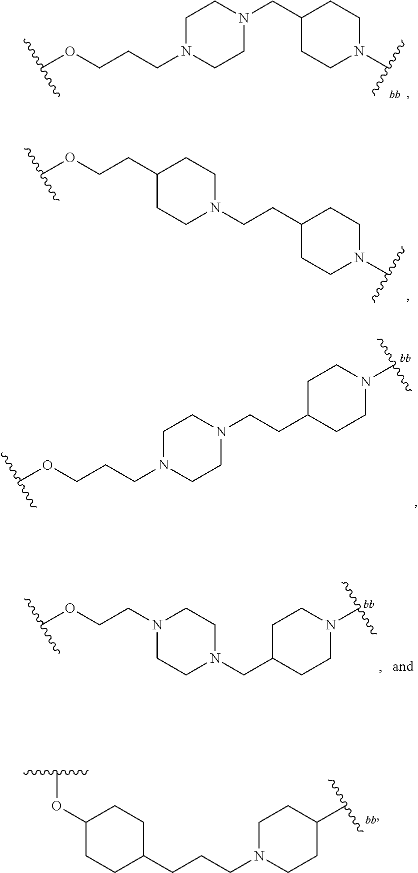

wherein bb represents the point of attachment to Ring C.

In some embodiments, L is: -(L^{A3})₀₋₁-(L^{A1})₀₋₅-L^{A4}-(L^{A1})₀₋₅-L^{A4}-L^{A3}-_{bb}; provided that L contains 2-7 L^{A1}; and wherein bb represents the point of attachment to Ring C. In some embodiments, L is: -(L^{A3})₀₋₁-(L^{A1})₀₋₅-L^{A4}-(L^{A1})₀₋₅-(4-10 (e.g., 6) membered heterocyclylene)-O— bb, provided that L contains 2-7 L^{A1}; wherein the 4-10 (e.g., 6) membered heterocyclylene is optionally substituted with 1-3 R^{a}; and wherein bb represents the point of attachment to Ring C. In some embodiments, 0-1 occurrence of L^{A1} is —CHR^{L}— or —C(R^{L})₂—; and each remaining occurrence of L^{A1} is —CH₂—. For example, L can be

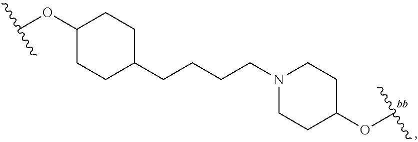

wherein bb represents the point of attachment to Ring C.

In some embodiments, L is a divalent group of Formula (L-3a) or (L-3b):

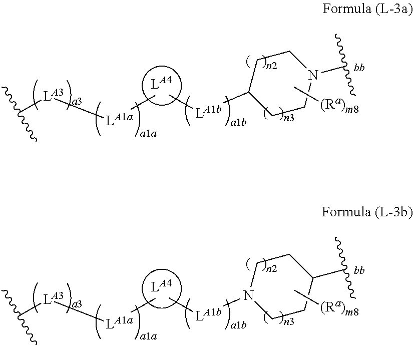

wherein:
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
n2 and n3 are independently 0, 1, or 2;
m8 is 0, 1, or 2; and
bb represents the point of attachment to Ring C.

In some embodiments, L is a divalent group of Formula (L-3c):

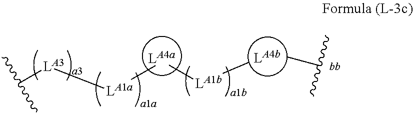

wherein:
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4a} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
n2 and n3 are independently 0, 1, or 2;
m8 is 0, 1, or 2;
L^{A4b} is 7-10 membered bicyclic nitrogen-containing heterocyclylene optionally substituted with 1-3 R^{a}; and
bb represents the point of attachment to Ring C.

In some embodiments of Formula (L-3c), L^{A4b} is a 7-10 membered spirocyclic nitrogen-containing heterocyclylene optionally substituted with 1-3 R^{a}. For example, L^{A4b} can be

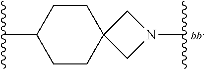 [SMILES: *C1CCC2(CC1)CN(*)C2]

In some embodiments of Formula (L-3c), L^{A4a} is a 4-10 membered heterocyclylene optionally substituted with 1-3 R^{a}.

In some embodiments, L is a divalent group of Formula (L-3d) or (L-4a):

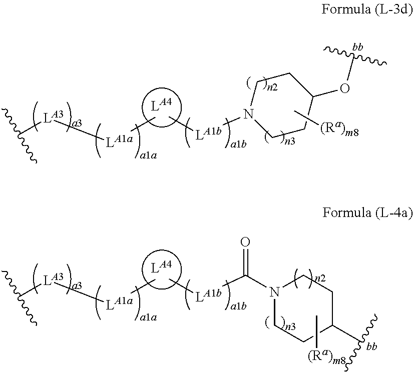

wherein:
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
n2 and n3 are independently 0, 1, or 2;
m8 is 0, 1, or 2; and
bb represents the point of attachment to Ring C.

In some embodiments of (L-3a), (L-3b), (L-3c), (L-3d), or (L-4a), a3 is 1. In some embodiments, L^{A3} is O. In some embodiments of (L-3a), a3 is 1; and L^{A3} is —O—.

In some embodiments of (L-3a), (L-3b), (L-3c), (L-3d), or (L-4a), a1a+a1b is 3 or 4. For example, a1a+a1b can be 3. For example, a1a+a1b can be 4.

In some embodiments of (L-3a), (L-3b), (L-3c), (L-3d), or (L-4a), a1a+a1b is 5.

In some embodiments of (L-3a), (L-3b), (L-3c), (L-3d), or (L-4a), a1a+a1b is 2.

In some embodiments of (L-3a), (L-3b), (L-3c), (L-3d), or (L-4a), each occurrence of L^{A1a} and L^{A1b} is —CH₂—. In some embodiments of (L-3a), (L-3b), (L-3c), (L-3d), or (L-4a), one occurrence of L^{A1a} is —CHR^{L}— or —C(R^{L})₂—; each remaining occurrence of L^{A1a} is —CH₂—; and each occurrence of L^{A1b} is —CH₂—. In some embodiments of (L-3a), (L-3b), (L-3c), (L-3d), or (L-4a), one occurrence of L^{A1b} is —CHR^{L}— or —C(R^{L})₂—; each remaining occurrence of L^{A1b} is —CH₂—; and each occurrence of L^{A1a} is —CH₂—.

In some embodiments of (L-3a), (L-3b), or (L-4a), m8 is 0. In some embodiments of (L-3a), (L-3b), (L-3c), or (L-4a), m8 is 1. In some embodiments of (L-3a), (L-3b), (L-3c), or (L-4a), m8 is 2.

In some embodiments of (L-3a), (L-3b), (L-3d), or (L-4a), n2 is 1; and n3 is 1. In some embodiments of (L-3a), (L-3b), (L-3d), or (L-4a), n2 is 0; and n3 is 1.

In some embodiments, L is: -(L^{A3})₀₋₁-(L^{A1})₂₋₃-L^{A3}-L^{A4}-(L^{A1})₀₋₅-(L^{A3})₁₋₂-_{bb}; provided that L contains 2-7 L^{A1}; and wherein bb represents the point of attachment to Ring C. In some embodiments, L is: -(L^{A})₀₋₁-CH₂CH₂—O-L^{A4}-(L^{A1})₀₋₅-(L^{A3})₁₋₂-_{bb}; wherein bb represents the point of attachment to Ring C. In some embodiments, L^{A4} is selected from the group consisting of: C₃₋₁₀ cycloalkylene or 4-10 membered heterocyclylene, each of which is optionally substituted with 1-3 R^{a}; and phenylene or 5-6 membered heteroarylene, each of which is optionally substituted with 1-3 R^{a}. In some embodiments, 0-1 occurrence of L^{A1} is —CHR^{L}— or —C(R^{L})₂—; and each remaining occurrence of L^{A1} is —CH₂—. For example, L can be

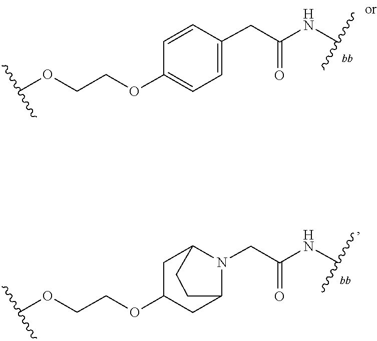

wherein bb represents the point of attachment to Ring C.

In some embodiments, L is a divalent group of Formula (L-5):

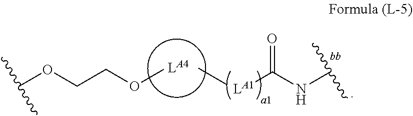

wherein:
a1 is an integer from 0 to 3;
L^{A4} is selected from the group consisting of:
a) C₃₋₁₀ cycloalkylene or 4-10 membered heterocyclylene, each of which is optionally substituted with 1-3 R^{a};
b) phenylene or 5-6 membered heteroarylene, each of which is optionally substituted with 1-3 R^{a}; and
bb represents the point of attachment to Ring C.

In some embodiments of (L-5), at is 1.

In some embodiments of (L-5), 0-1 occurrence of L^{A1} is —CHR^{L}— or —C(R^{L})₂—; and each remaining occurrence of L^{A1} is —CH₂—.

In some embodiments, L is: -(L^{A1})₁₋₅-L^{A3}-L^{A4}-(L^{A1})₀₋₅-(L^{A3})₁₋₂-_{bb}; provided that L contains 2-7 L^{A1}; and wherein bb represents the point of attachment to Ring C. In some embodiments, L is: -(L^{A1})₂₋₃-O-L^{A4}-(L^{A1})₀₋₅-C(═O)N(R^{d})-_{bb}; provided that L contains 2-7 L^{A1}; and wherein bb represents the point of attachment to Ring C. In some embodiments, L^{A4} is 4-10 membered nitrogen-containing heterocyclylene optionally substituted with 1-3 R^{a}. In some embodiments, 0-1 occurrence of L^{A1} is —CHR^{L}— or —C(R^{L})₂—; and each remaining occurrence of L^{A1} is —CH₂—. For example, L can be

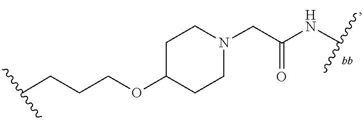

wherein bb represents the point of attachment to Ring C.

In some embodiments, L is a divalent group of Formula (L-5a):

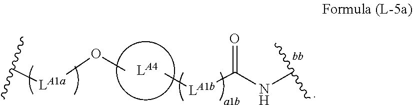

wherein:
a1a and a1b are independently 1, 2, or 3;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
bb represents the point of attachment to Ring C.

In some embodiments of Formula (L-5a), a1a is 3. In some embodiments of Formula (L-5a), a1b is 1.

In some embodiments of Formula (L-5a), each occurrence of L^{A1a} and L^{A1b} is —CH₂—. In some embodiments of Formula (L-5a), one occurrence of L^{A1a} is —CHR^{L}— or —C(R^{L})₂—; each remaining occurrence of L^{A1a} is —CH₂—; and each occurrence of L^{A1b} is —CH₂—. In some embodiments of Formula (L-5a), one occurrence of L^{A1b} is —CHR^{L}— or —C(R^{L})₂—; each remaining occurrence of L^{A1b} is —CH₂—; and each occurrence of L^{A1a} is —CH₂—.

In some embodiments of (L-1), (L-2), (L-2a), (L-3), (L-3a), (L-3b), (L-3d), (L-4), (L-4a), (L-5) or (L-5a), L^{A4} is 4-10 membered heterocyclylene optionally substituted with 1-3 R^{a}. In some embodiments, L^{A4} is 4-10 membered nitrogen-containing heterocyclylene optionally substituted with 1-3 R^{a}.

In some embodiments of (L-1), (L-2), (L-2a), (L-3), (L-3a), (L-3b), (L-3d), (L-4), (L-4a), (L-5) or (L-5a), L^{A4} is selected from the group consisting of:

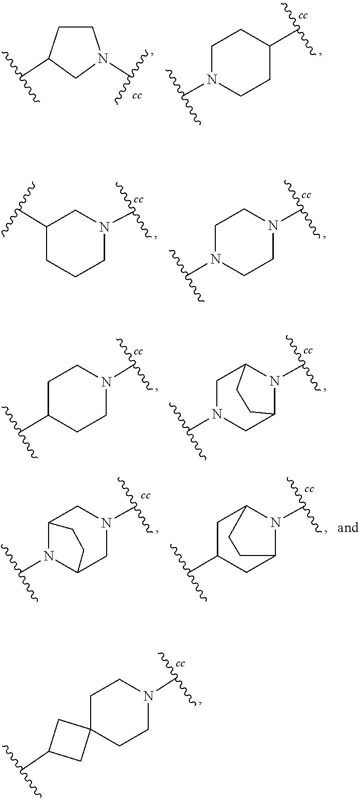

each optionally substituted with 1-3 R^{a} at one or more ring carbon atoms, wherein cc represents the point of attachment to L^{A1b} or L^{A1}. In some embodiments, each R^{a} present on L^{A4} is independently F or C₁₋₃ alkyl optionally substituted with 1-3 F.

In some embodiments of (L-1), (L-2), (L-2a), (L-3), (L-3a), (L-3b), (L-3d), (L-4), (L-4a), (L-5) or (L-5a), L^{A4} is C₃₋₁₀ cycloalkylene optionally substituted with 1-3 R^{a}. For example, L^{A4} can be 1,4-cyclohexylene optionally substituted with 1-3 R^{a}.

In some embodiments of (L-1), (L-2), (L-2a), (L-3), (L-3a), (L-3b), (L-3d), (L-4), (L-4a), (L-5) or (L-5a), L^{A4} is phenylene or 5-6 membered heteroarylene, each of which is optionally substituted with 1-3 R^{a}. In some embodiments, L^{A4} is phenylene optionally substituted with 1-3 R^{a}. In some embodiments, L^{A4} is 1,2-phenylene optionally substituted with 1-3 R^{a}. In some embodiments, L^{A4} is 1,4-phenylene optionally substituted with 1-3 R^{a}. In some embodiments, L^{A4} is 1,3-phenylene optionally substituted with 1-3 R^{a}.

In some embodiments, L is -(L^{A3})₀₋₁-(L^{A1})₂₋₉-L^{A3})₀₋₁-(piperazinylene)-_{bb}, wherein bb represents the point of attachment to Ring C. In some embodiments, 0-1 occurrence of L^{A1} is —CHR^{L}— or —C(R^{L})₂—; and each remaining occurrence of L^{A1} is —CH₂—.

In some embodiments, L is a divalent group of Formula (L-6):

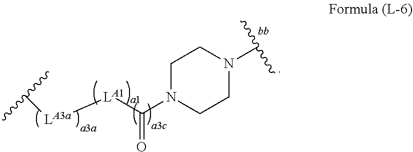

wherein:
L^{A3a} is selected from the group consisting of: —O—, —N(H)—, and —N(C₁₋₃ alkyl)-;
a3a is 0 or 1;
at is an integer from 2 to 11;
a3c is 0 or 1; and
bb represents the point of attachment to Ring C.

In some embodiments of (L-6), a3a is 1. In some embodiments, L^{A3a} is —O—. In some embodiments of (L-6), a3a is 1 and L^{A3a} is —O—.

In some embodiments of (L-6), 0-1 occurrence of L^{A1} is —CHR^{L}— or —C(R^{L})₂—; and each remaining occurrence of L^{A1} is —CH₂—.

In some embodiments of (L-6), at is an integer from 2 to 5. In some embodiments of (L-6), at is an integer from 5 to 7 (e.g., 6). In some embodiments of (L-6), at is an integer from 7 to 9 (e.g., 8). In some embodiments of (L-6), at is an integer from 9 to 11.

In some embodiments, L is selected from the group consisting of:

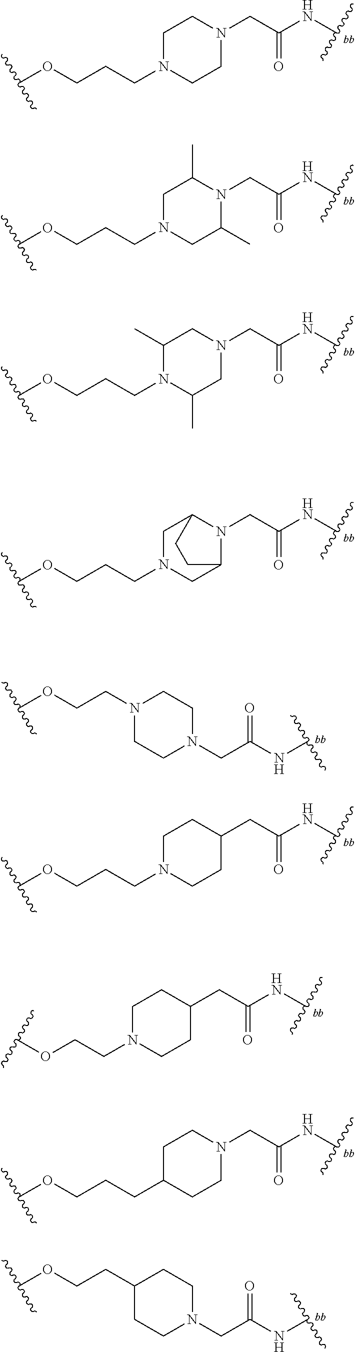

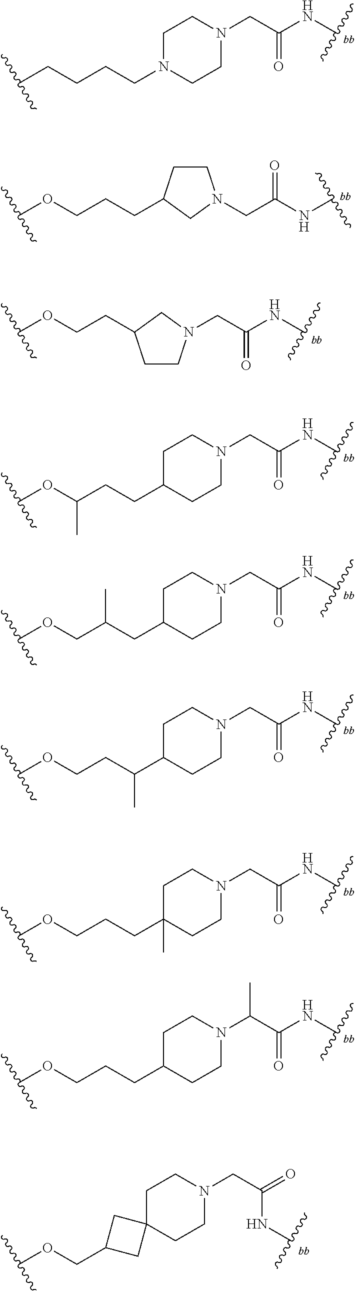

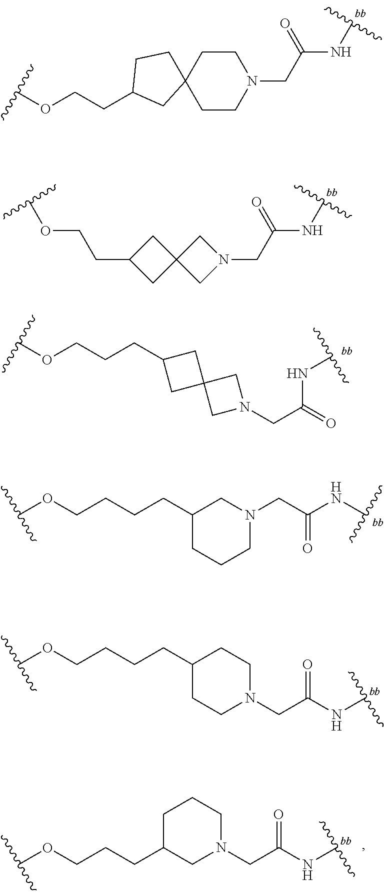

wherein bb represents the point of attachment to Ring C.

In some embodiments, L is selected from the group consisting of:

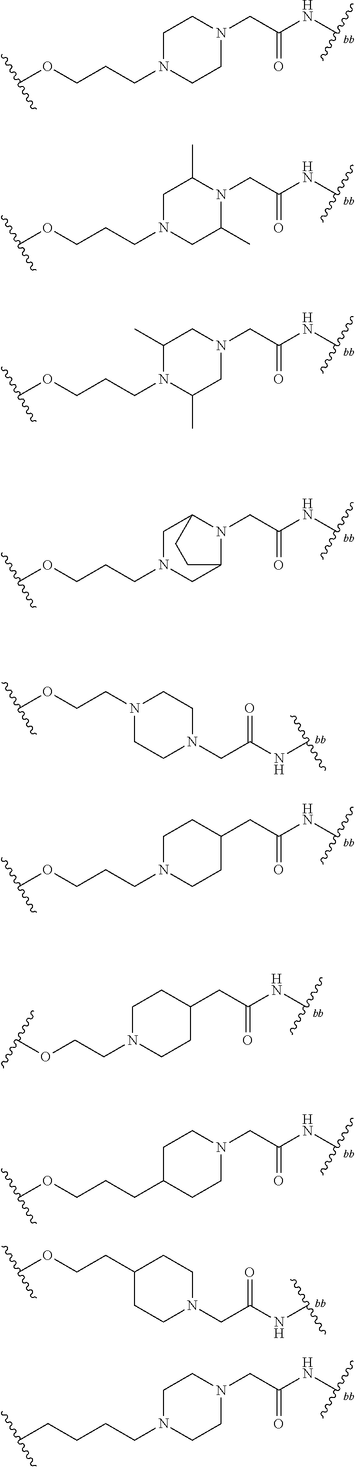

wherein bb represents the point of attachment to Ring C.

In some embodiments, L is selected from the group consisting of:

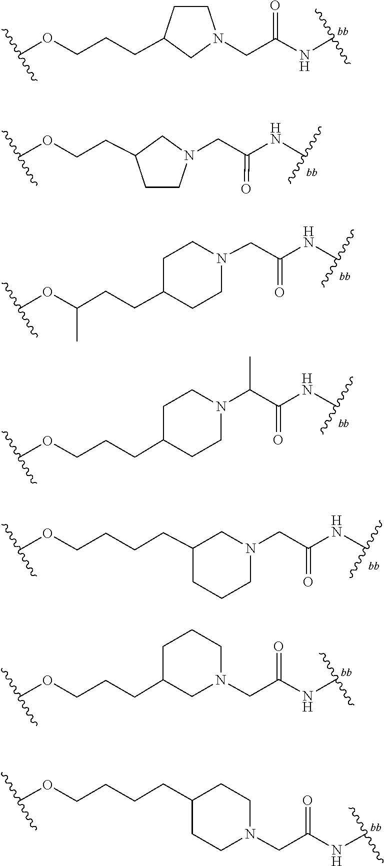

wherein bb represents the point of attachment to Ring C.

In some embodiments, L is selected from the group consisting of:

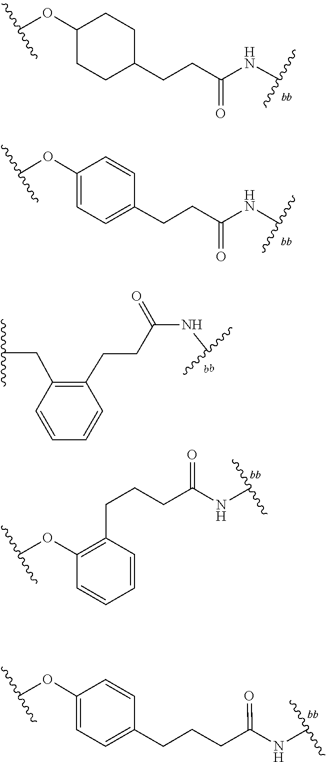

wherein bb represents the point of attachment to Ring C.

In some embodiments, L is

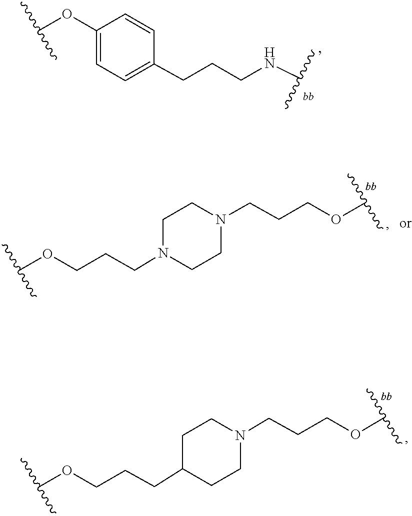

wherein bb represents the point of attachment to Ring C.

In some embodiments, L is selected from the group consisting of

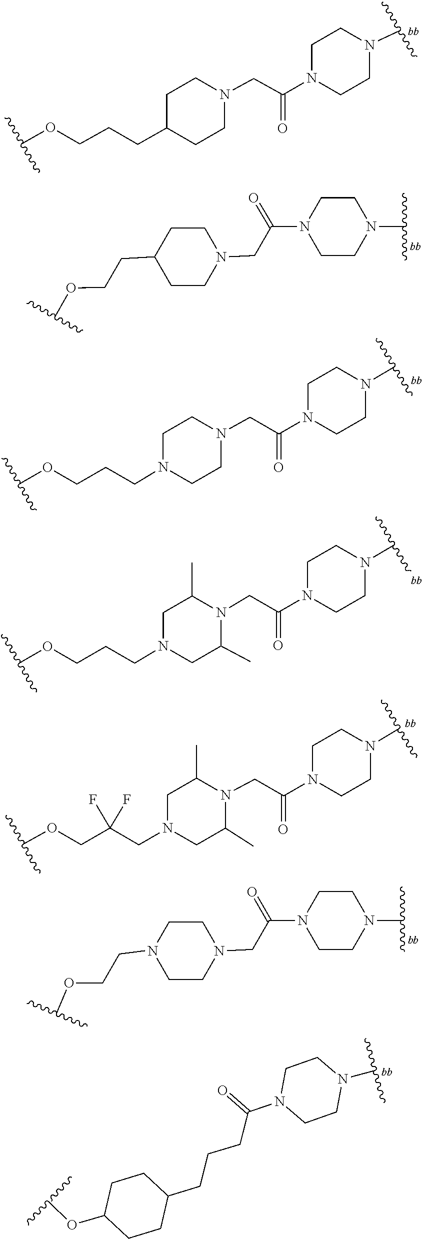

wherein bb represents the point of attachment to Ring C.

In some embodiments, L is selected from the group consisting of:

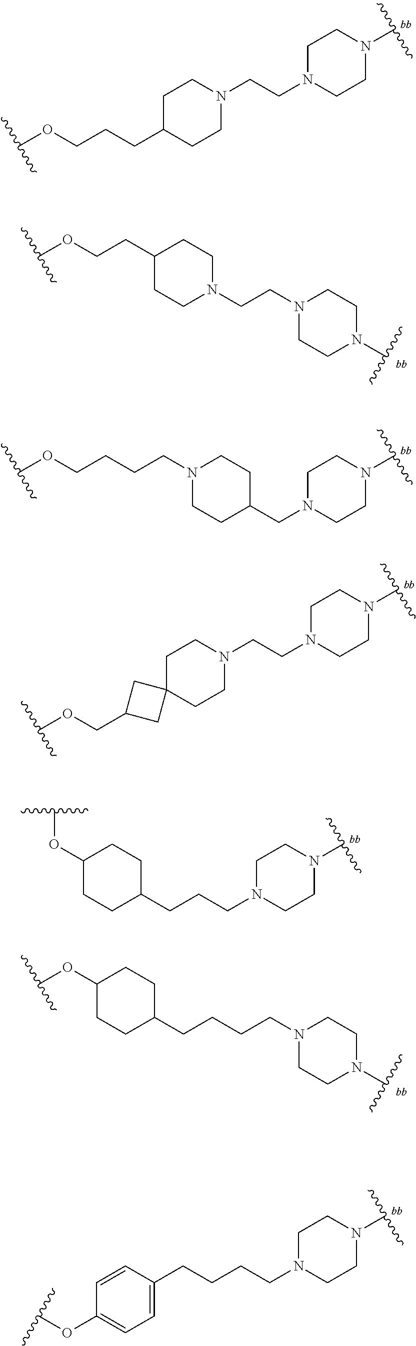

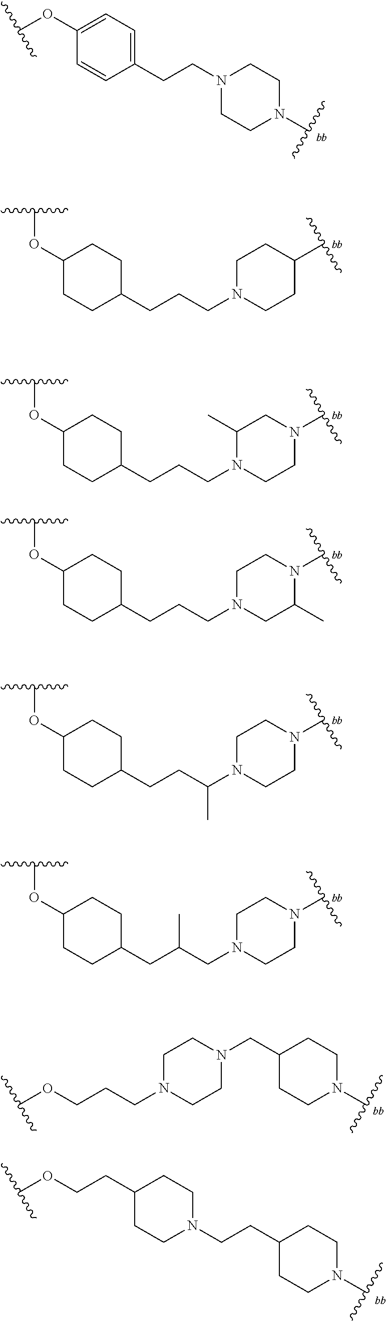

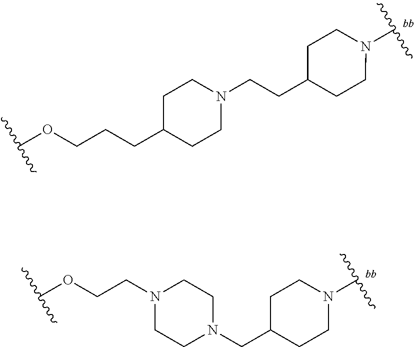

wherein bb represents the point of attachment to Ring C.

In some embodiments, L is

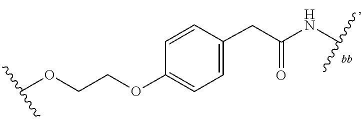

wherein bb represents the point of attachment to Ring C. In some embodiments, L is

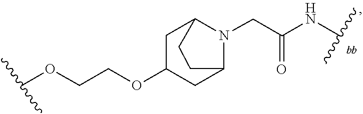

wherein bb represents the point of attachment to Ring C.

In some embodiments, L is -(L^{A})ₙ₁-; and L^{A} and n1 are defined according to (BB). In some embodiments, n1 is an integer from 3 to 5. In some embodiments, n1 is an integer from 5 to 9. In some embodiments, n1 is an integer from 9 to 15. In some embodiments, 1-4 occurrences of L^{A} is L^{A3}. In some embodiments, 1-3 occurrences of L^{A} is L^{A3}. In some embodiments, 2-3 occurrences of L^{A} are L^{A3}. In some embodiments, 0-1 occurrence of L^{A3} is C(═O); and each remaining occurrence of L^{A3} is independently selected from the group consisting of: —O—; —N(H)—; and —N(C₁₋₃ alkyl)-. In some embodiments, 0-2 occurrences of L^{A1} is —CHR^{L}— or —C(R^{L})₂—; and each remaining occurrence of L^{A1} is —CH₂—.

In some embodiments, L^{A} and n1 are defined according to (BB); and n1 is an integer from 3 to 5. In some embodiments, L^{A} and n1 are defined according to (BB); and n1 is an integer from 9 to 15.

In some embodiments, L^{A} and n1 are defined according to (BB); and L is -(L^{A3})₀₋₂-(L^{A1})₁₋₁₅-(L^{A3})₀₋₂-_{bb}, wherein bb represents the point of attachment to Ring C. In some embodiments, L is -(L^{A3})₀₋₁-(L^{A1})₁₋₁₅-(L^{A3})₁₋₂-_{bb}, wherein bb represents the point of attachment to Ring C. In some embodiments, L contains 2 or 3 L^{A3}. In some embodiments, 0-1 occurrence of L^{A3} is C(═O); and each remaining occurrence of L^{A3} is independently selected from the group consisting of: —O—; —N(H)—; and —N(C₁₋₃ alkyl)-. In some embodiments, 0-2 occurrences of L^{A1} is —CHR^{L}— or —C(R^{L})₂—; and each remaining occurrence of L^{A1} is —CH₂—. In some embodiments, each occurrence of L^{A1} is —CH₂—. In some embodiments, one occurrence of L^{A1} is —CHR^{L}— or —C(R^{L})₂—; and each remaining occurrence of L^{A1} is —CH₂—. In some embodiments, each R^{L} is independently selected from the group consisting of: —F and —C₁₋₃ alkyl optionally substituted with 1-3 F.

In some embodiments, L is a divalent group of Formula (L-7):

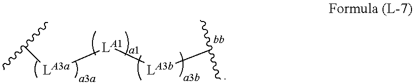

wherein:
L^{A3a} is selected from the group consisting of: —O—, —N(H)—, and —N(C₁₋₃ alkyl)-;
a3a is 0 or 1;
a1 is an integer from 1 to 15;
each L^{A3b} is independently selected from the group consisting of: C(═O), —O—, N(H)—, and —N(C₁₋₃ alkyl)-;
a3b is 1 or 2; and
bb represents the point of attachment to Ring C.

In some embodiments of (L-7), -(L^{A3b})_{a3b}- is

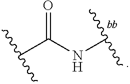 [SMILES: *NC(*)=O]

In some embodiments of (L-7), -(L^{A3b})_{a3b}- is —N(H)— or —N(C₁₋₃ alkyl)-.

In some embodiments of (L-7), 0-2 occurrences of L^{A1} is —CHR^{L}— or —C(R^{L})₂—; and each remaining occurrence of L^{A1} is —CH₂—. In some embodiments, each occurrence of L^{A1} is —CH₂—.

In some embodiments, L is (L-1); Ring A is

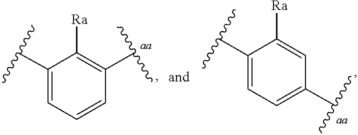

wherein aa represents the point of attachment to L, wherein the R^{a} present on Ring A is methyl or CF₃; and R¹ is C(O)OH or C(O)OC₁₋₆ alkyl.

In some embodiments, L is (L-3); Ring A is

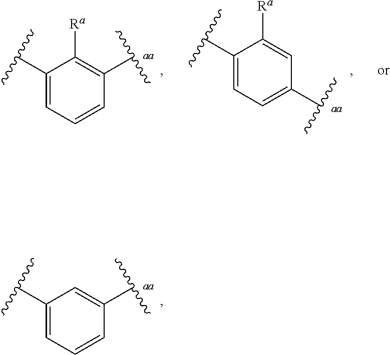

wherein aa represents the point of attachment to L, wherein the R^{a} present on Ring A is methyl or CF₃; and R¹ is C(O)OH or C(O)OC₁₋₆ alkyl.

In some embodiments, L is (L-3a); Ring A is

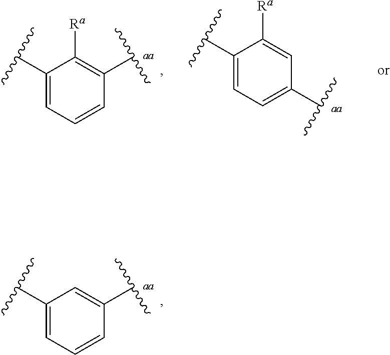

wherein aa represents the point of attachment to L, wherein the R^{a} present on Ring A is methyl or CF₃; and R¹ is C(O)OH or C(O)OC₁₋₆ alkyl.

In some embodiments, L is (L-3b); Ring A is

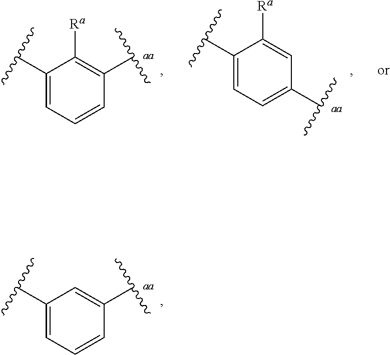

wherein aa represents the point of attachment to L, wherein the R^{a} present on Ring A is methyl or CF₃; and R¹ is C(O)OH or C(O)OC₁₋₆ alkyl.

In some embodiments, Ring A is

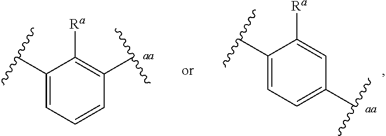

wherein aa represents the point of attachment to L, wherein the R^{a} present on Ring A is methyl or CF₃; R¹ is C(O)OH or C(O)OC₁₋₆ alkyl; and Ring C is

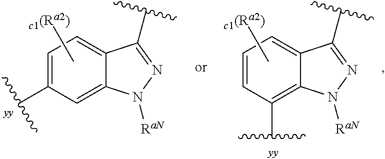

wherein R^{aN} is C₁₋₃ alkyl (e.g., methyl); c1 is 0 or 1; and R^{a2} is selected from the group consisting of halo (e.g., —F) and C₁₋₃ alkyl optionally substituted with 1-3 F.

In some embodiments of Formula (I), L is (L-1); Ring A is

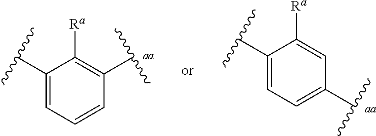

wherein aa represents the point of attachment to L, wherein the R^{a} present on Ring A is methyl or CF₃; R¹ is C(O)OH or C(O)OC₁₋₆ alkyl; and Ring C is

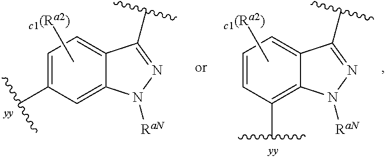

wherein R^{aN} is C₁₋₃ alkyl (e.g., methyl); c1 is 0 or 1; and R^{a2} is selected from the group consisting of halo (e.g., —F) and C₁₋₃ alkyl optionally substituted with 1-3 F.

In some embodiments of Formula (I), L is (L-3); Ring A is

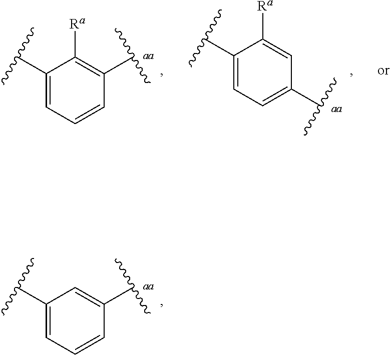

wherein aa represents the point of attachment to L, wherein the R^{a} present on Ring A is methyl or CF₃; R¹ is C(O)OH or C(O)OC₁₋₆ alkyl; and Ring C is

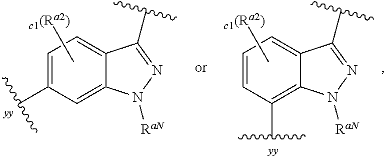

wherein R^{aN} is C₁₋₃ alkyl (e.g., methyl); c1 is 0 or 1; and R^{a2} is selected from the group consisting of halo (e.g., —F) and C₁₋₃ alkyl optionally substituted with 1-3 F.

In some embodiments of Formula (I), L is (L-3a); Ring A is

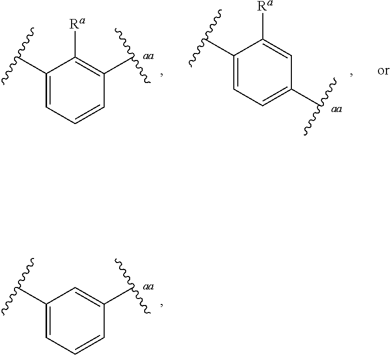

wherein aa represents the point of attachment to L, wherein the R^{a} present on Ring A is methyl or CF₃; R¹ is C(O)OH or C(O)OC₁₋₆ alkyl; and Ring C is

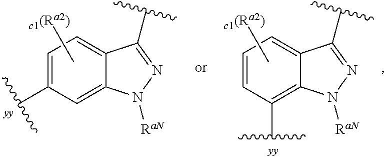

wherein R^{aN} is C₁₋₃ alkyl (e.g., methyl); c1 is 0 or 1; and R^{a2} is selected from the group consisting of halo (e.g., —F) and C₁₋₃ alkyl optionally substituted with 1-3 F.

In some embodiments of Formula (I), L is (L-3b); Ring A is

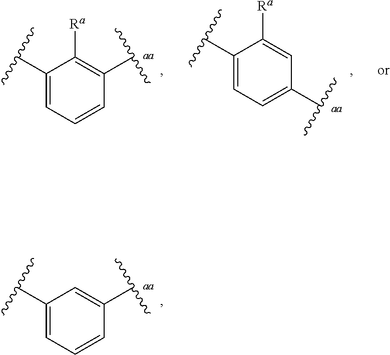

wherein aa represents the point of attachment to L, wherein the R^{a} present on Ring A is methyl or CF₃; R¹ is C(O)OH or C(O)OC₁₋₆ alkyl; and Ring C is

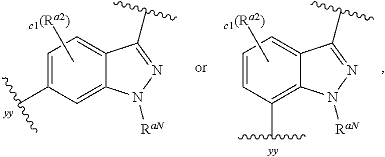

wherein R^{aN} is C₁₋₃ alkyl (e.g., methyl); c1 is 0 or 1; and R^{a2} is selected from the group consisting of halo (e.g., —F) and C₁₋₃ alkyl optionally substituted with 1-3 F.

In some embodiments, L is selected from the group consisting of the moieties depicted in Table (L), wherein bb represents the point of attachment to Ring C:

TABLE (L)
Examples when L is (L-1)

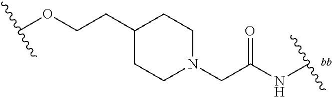 [SMILES: *NC(=O)CN1CCC(CCO*)CC1]

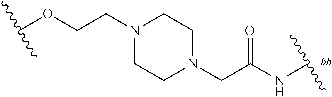 [SMILES: *NC(=O)CN1CCN(CCO*)CC1]

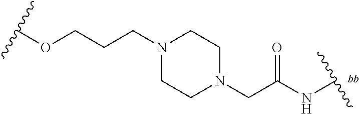 [SMILES: *NC(=O)CN1CCN(CCCO*)CC1]

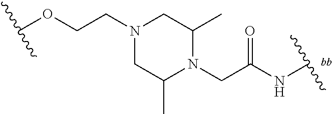 [SMILES: *NC(=O)CN1C(C)CN(CCO*)CC1C]

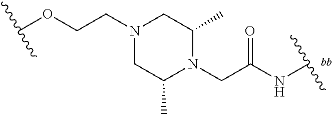 [SMILES: *NC(=O)CN1[C@H](C)CN(CCO*)C[C@@H]1C]

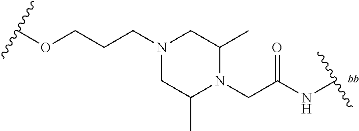 [SMILES: *NC(=O)CN1C(C)CN(CCCO*)CC1C]

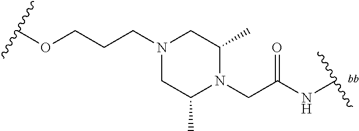 [SMILES: *NC(=O)CN1[C@H](C)CN(CCCO*)C[C@@H]1C]

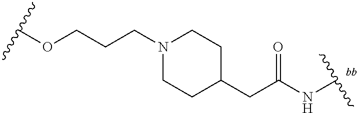 [SMILES: *NC(=O)CC1CCN(CCCO*)CC1]

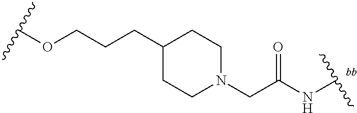 [SMILES: *NC(=O)CN1CCC(CCCO*)CC1]

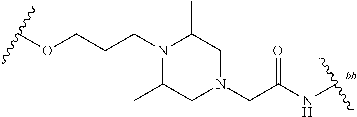 [SMILES: *NC(=O)CN1CC(C)N(CCCO*)C(C)C1]

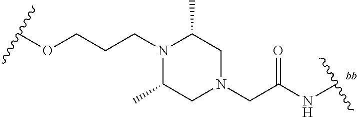 [SMILES: *NC(=O)CN1C[C@@H](C)N(CCCO*)[C@@H](C)C1]

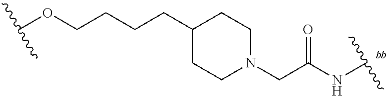 [SMILES: *NC(=O)CN1CCC(CCCCO*)CC1]

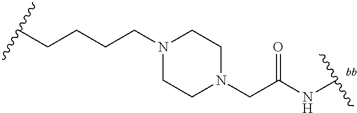 [SMILES: *CCCCN1CCN(CC(=O)N*)CC1]

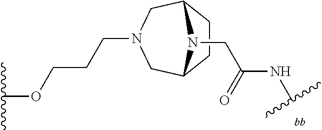 [SMILES: *NC(=O)CN1[C@@H]2CC[C@H]1CN(CCCO*)C2]

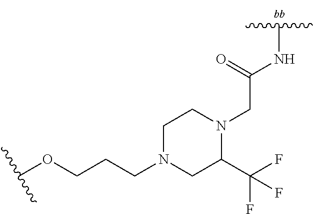 [SMILES: *NC(=O)CN1CCN(CCCO*)CC1C(F)(F)F]

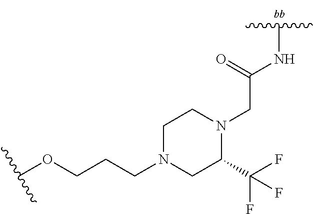 [SMILES: *NC(=O)CN1CCN(CCCO*)C[C@H]1C(F)(F)F]

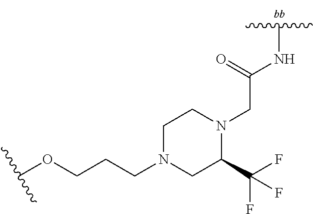 [SMILES: *NC(=O)CN1CCN(CCCO*)C[C@@H]1C(F)(F)F]

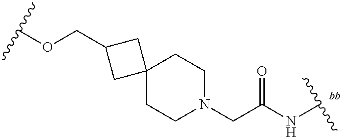 [SMILES: *NC(=O)CN1CCC2(CC1)CC(CO*)C2]

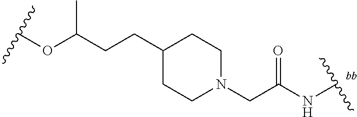 [SMILES: *NC(=O)CN1CCC(CCC(C)O*)CC1]

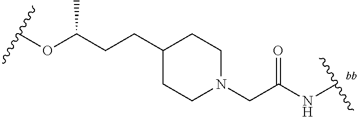 [SMILES: *NC(=O)CN1CCC(CC[C@@H](C)O*)CC1]

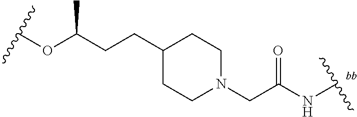 [SMILES: *NC(=O)CN1CCC(CC[C@H](C)O*)CC1]

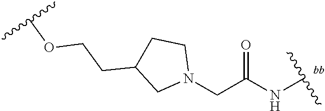 [SMILES: *NC(=O)CN1CCC(CCO*)C1]

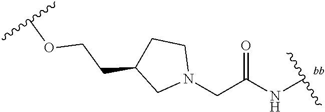 [SMILES: *NC(=O)CN1CC[C@H](CCO*)C1]

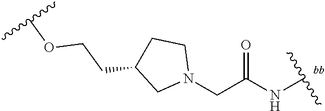 [SMILES: *NC(=O)CN1CC[C@@H](CCO*)C1]

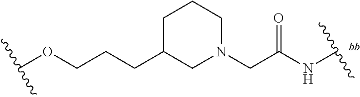 [SMILES: *NC(=O)CN1CCCC(CCCO*)C1]

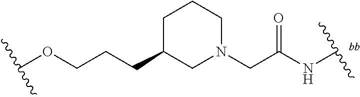 [SMILES: *NC(=O)CN1CCC[C@H](CCCO*)C1]

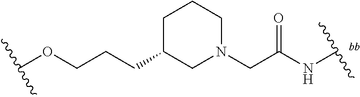 [SMILES: *NC(=O)CN1CCC[C@@H](CCCO*)C1]

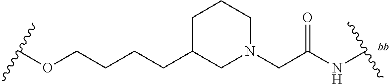 [SMILES: *NC(=O)CN1CCCC(CCCCO*)C1]

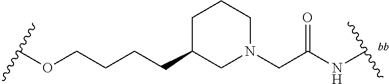 [SMILES: *NC(=O)CN1CCC[C@H](CCCCO*)C1]

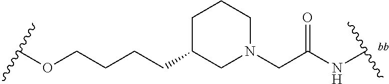 [SMILES: *NC(=O)CN1CCC[C@@H](CCCCO*)C1]

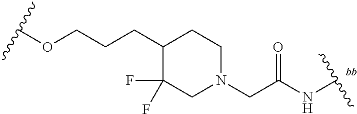 [SMILES: *NC(=O)CN1CCC(CCCO*)C(F)(F)C1]

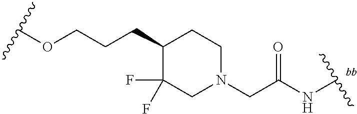 [SMILES: *NC(=O)CN1CC[C@H](CCCO*)C(F)(F)C1]

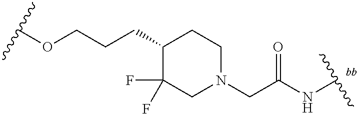 [SMILES: *NC(=O)CN1CC[C@@H](CCCO*)C(F)(F)C1]

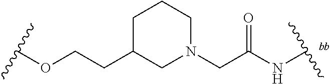 [SMILES: *NC(=O)CN1CCCC(CCO*)C1]

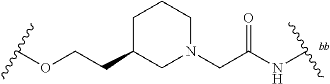 [SMILES: *NC(=O)CN1CCC[C@H](CCO*)C1]

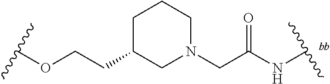 [SMILES: *NC(=O)CN1CCC[C@@H](CCO*)C1]

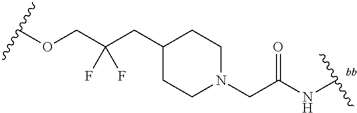 [SMILES: *NC(=O)CN1CCC(CC(F)(F)CO*)CC1]

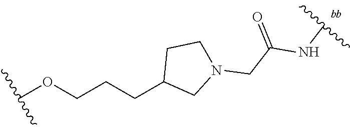 [SMILES: *NC(=O)CN1CCC(CCCO*)C1]

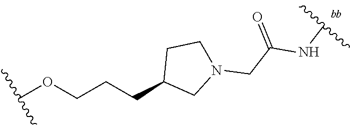 [SMILES: *NC(=O)CN1CC[C@H](CCCO*)C1]

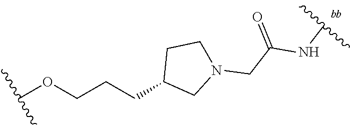 [SMILES: *NC(=O)CN1CC[C@@H](CCCO*)C1]

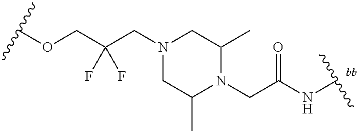 [SMILES: *NC(=O)CN1C(C)CN(CC(F)(F)CO*)CC1C]

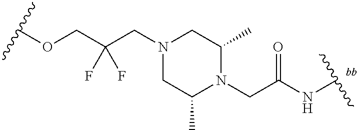 [SMILES: *NC(=O)CN1[C@H](C)CN(CC(F)(F)CO*)C[C@@H]1C]

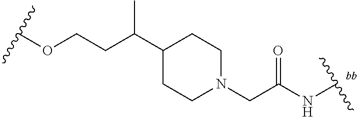 [SMILES: *NC(=O)CN1CCC(C(C)CCO*)CC1]

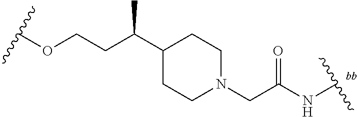 [SMILES: *NC(=O)CN1CCC([C@H](C)CCO*)CC1]

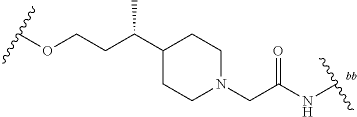 [SMILES: *NC(=O)CN1CCC([C@@H](C)CCO*)CC1]

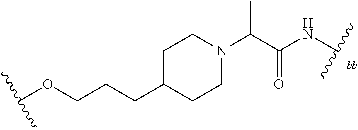 [SMILES: *NC(=O)C(C)N1CCC(CCCO*)CC1]

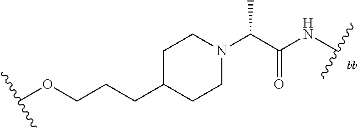 [SMILES: *NC(=O)[C@@H](C)N1CCC(CCCO*)CC1]

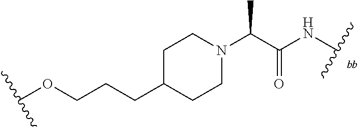 [SMILES: *NC(=O)[C@H](C)N1CCC(CCCO*)CC1]

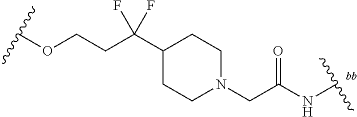 [SMILES: *NC(=O)CN1CCC(C(F)(F)CCO*)CC1]

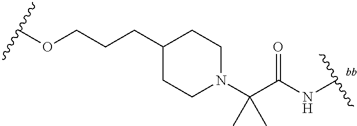 [SMILES: *NC(=O)C(C)(C)N1CCC(CCCO*)CC1]

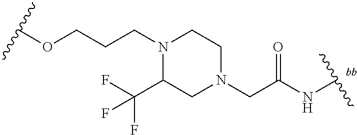 [SMILES: *NC(=O)CN1CCN(CCCO*)C(C(F)(F)F)C1]

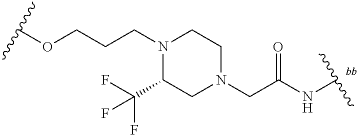 [SMILES: *NC(=O)CN1CCN(CCCO*)[C@@H](C(F)(F)F)C1]

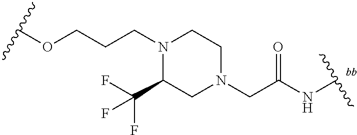 [SMILES: *NC(=O)CN1CCN(CCCO*)[C@H](C(F)(F)F)C1]

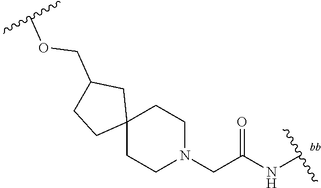 [SMILES: *NC(=O)CN1CCC2(CCC(CO*)C2)CC1]

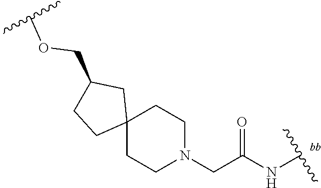 [SMILES: *NC(=O)CN1CCC2(CC[C@@H](CO*)C2)CC1]

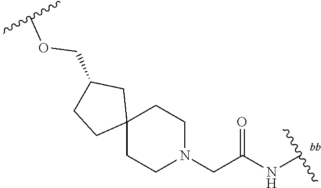 [SMILES: *NC(=O)CN1CCC2(CC[C@H](CO*)C2)CC1]

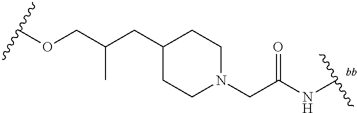 [SMILES: *NC(=O)CN1CCC(CC(C)CO*)CC1]

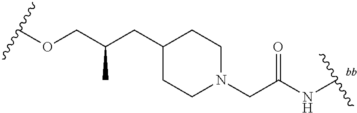 [SMILES: *NC(=O)CN1CCC(C[C@@H](C)CO*)CC1]

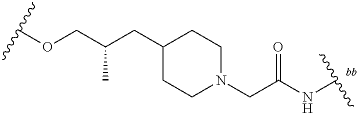 [SMILES: *NC(=O)CN1CCC(C[C@H](C)CO*)CC1]

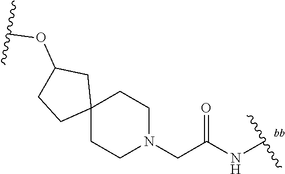 [SMILES: *NC(=O)CN1CCC2(CCC(O*)C2)CC1]

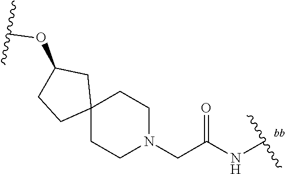 [SMILES: *NC(=O)CN1CCC2(CC[C@@H](O*)C2)CC1]

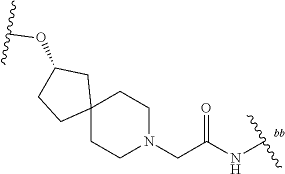 [SMILES: *NC(=O)CN1CCC2(CC[C@H](O*)C2)CC1]

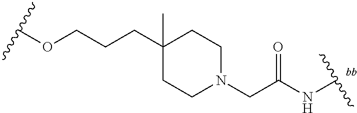 [SMILES: *NC(=O)CN1CCC(C)(CCCO*)CC1]

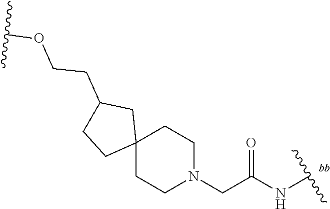 [SMILES: *NC(=O)CN1CCC2(CCC(CCO*)C2)CC1]

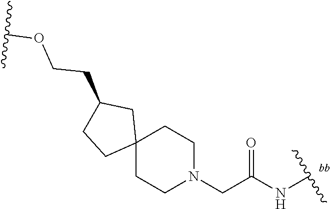 [SMILES: *NC(=O)CN1CCC2(CC[C@@H](CCO*)C2)CC1]

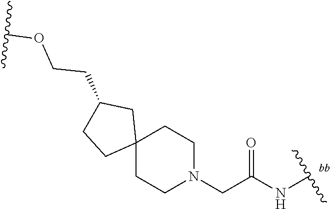 [SMILES: *NC(=O)CN1CCC2(CC[C@H](CCO*)C2)CC1]

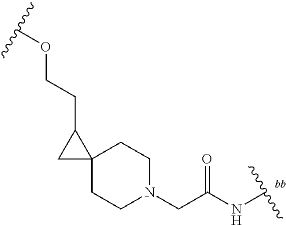 [SMILES: *NC(=O)CN1CCC2(CC1)CC2CCO*]

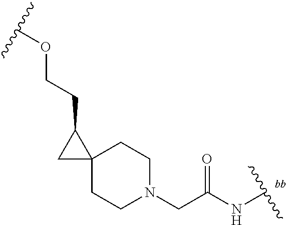 [SMILES: *NC(=O)CN1CCC2(CC1)C[C@H]2CCO*]

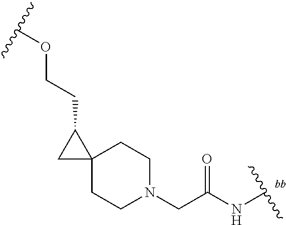 [SMILES: *NC(=O)CN1CCC2(CC1)C[C@@H]2CCO*]

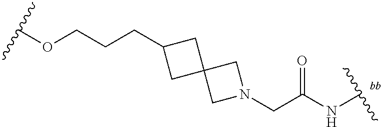 [SMILES: *NC(=O)CN1CC2(CC(CCCO*)C2)C1]

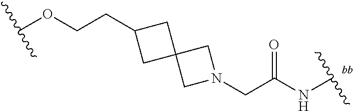 [SMILES: *NC(=O)CN1CC2(CC(CCO*)C2)C1]

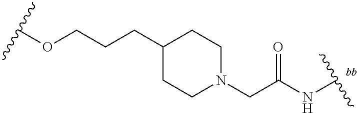 [SMILES: *NC(=O)CN1CCC(CCCO*)CC1]

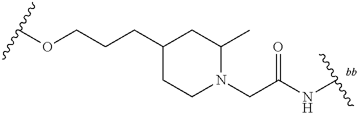 [SMILES: *NC(=O)CN1CCC(CCCO*)CC1C]

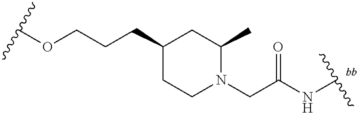 [SMILES: *NC(=O)CN1CC[C@@H](CCCO*)C[C@H]1C]

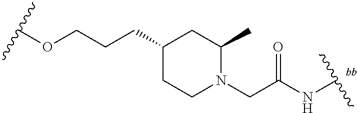 [SMILES: *NC(=O)CN1CC[C@H](CCCO*)C[C@H]1C]

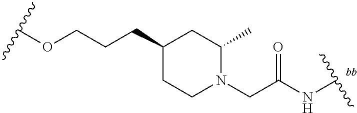 [SMILES: *NC(=O)CN1CC[C@@H](CCCO*)C[C@@H]1C]

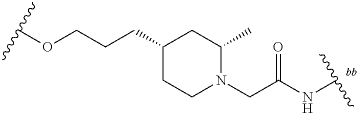 [SMILES: *NC(=O)CN1CC[C@H](CCCO*)C[C@@H]1C]

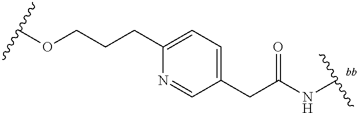 [SMILES: *NC(=O)Cc1ccc(CCCO*)nc1]

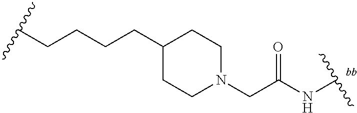 [SMILES: *CCCCC1CCN(CC(=O)N*)CC1]

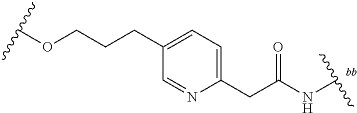 [SMILES: *NC(=O)Cc1ccc(CCCO*)cn1]

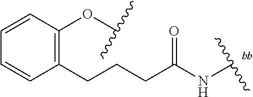 [SMILES: *NC(=O)CCCc1ccccc1O*]

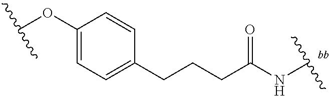 [SMILES: *NC(=O)CCCc1ccc(O*)cc1]

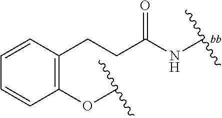 [SMILES: *NC(=O)CCc1ccccc1O*]

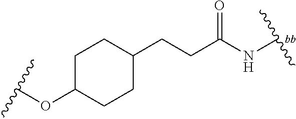 [SMILES: *NC(=O)CCC1CCC(O*)CC1]

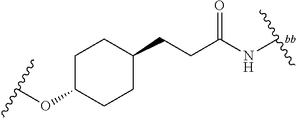 [SMILES: *NC(=O)CC[C@H]1CC[C@H](O*)CC1]

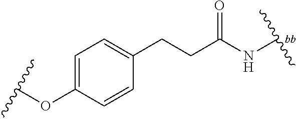 [SMILES: *NC(=O)CCc1ccc(O*)cc1]

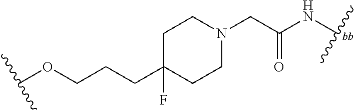 [SMILES: *NC(=O)CN1CCC(F)(CCCO*)CC1]

Examples when L is (L-2)

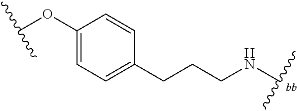 [SMILES: *NCCCc1ccc(O*)cc1]

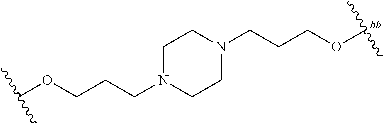 [SMILES: *OCCCN1CCN(CCCO*)CC1]

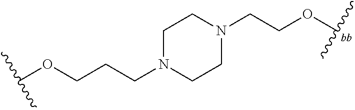 [SMILES: *OCCCN1CCN(CCO*)CC1]

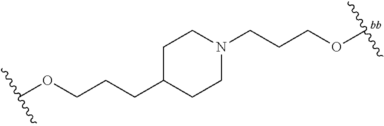 [SMILES: *OCCCC1CCN(CCCO*)CC1]

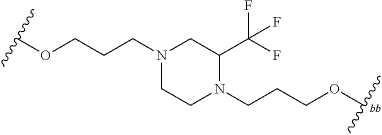 [SMILES: *OCCCN1CCN(CCCO*)C(C(F)(F)F)C1]

Examples when L is (L-2a)

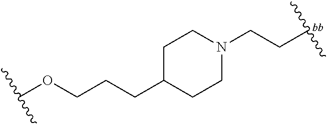 [SMILES: *CCN1CCC(CCCO*)CC1]

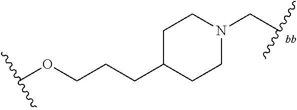 [SMILES: *CN1CCC(CCCO*)CC1]

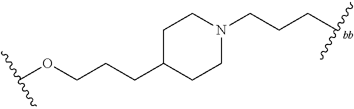 [SMILES: *CCCN1CCC(CCCO*)CC1]

Examples when L is (L-3)

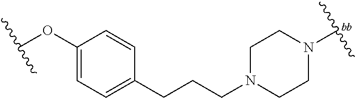 [SMILES: *Oc1ccc(CCCN2CCN(*)CC2)cc1]

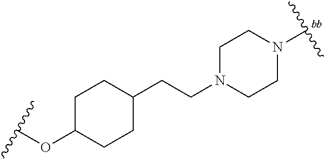 [SMILES: *OC1CCC(CCN2CCN(*)CC2)CC1]

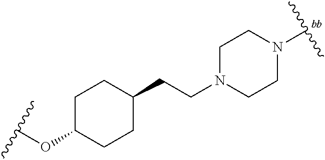 [SMILES: *O[C@H]1CC[C@H](CCN2CCN(*)CC2)CC1]

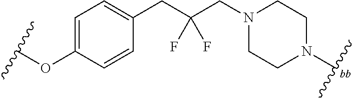 [SMILES: *Oc1ccc(CC(F)(F)CN2CCN(*)CC2)cc1]

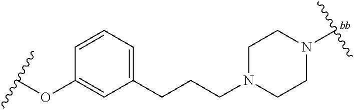 [SMILES: *Oc1cccc(CCCN2CCN(*)CC2)c1]

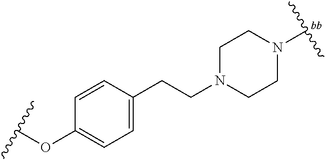 [SMILES: *Oc1ccc(CCN2CCN(*)CC2)cc1]

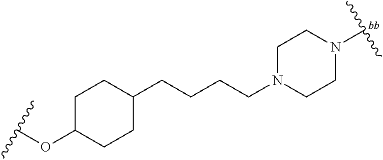 [SMILES: *OC1CCC(CCCCN2CCN(*)CC2)CC1]

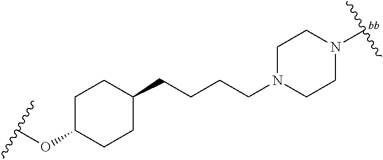 [SMILES: *O[C@H]1CC[C@H](CCCCN2CCN(*)CC2)CC1]

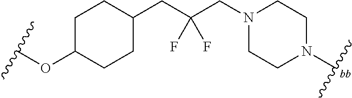 [SMILES: *OC1CCC(CC(F)(F)CN2CCN(*)CC2)CC1]

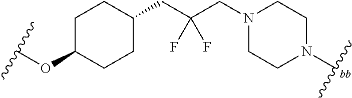 [SMILES: *O[C@H]1CC[C@H](CC(F)(F)CN2CCN(*)CC2)CC1]

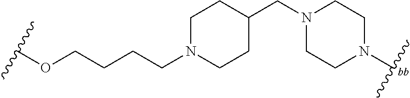 [SMILES: *OCCCCN1CCC(CN2CCN(*)CC2)CC1]

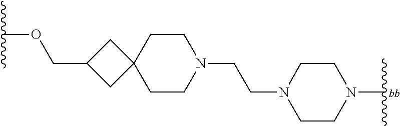 [SMILES: *OCC1CC2(CCN(CCN3CCN(*)CC3)CC2)C1]

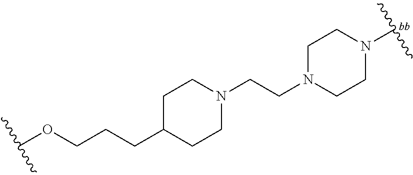 [SMILES: *OCCCC1CCN(CCN2CCN(*)CC2)CC1]

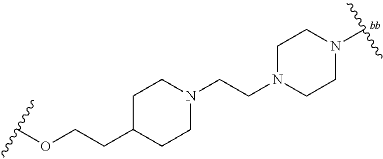 [SMILES: *OCCC1CCN(CCN2CCN(*)CC2)CC1]

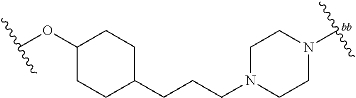 [SMILES: *OC1CCC(CCCN2CCN(*)CC2)CC1]

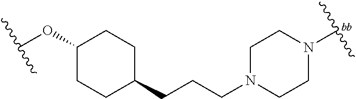 [SMILES: *O[C@H]1CC[C@H](CCCN2CCN(*)CC2)CC1]

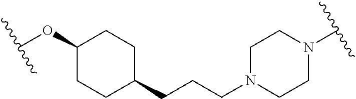 [SMILES: *O[C@H]1CC[C@@H](CCCN2CCN(*)CC2)CC1]

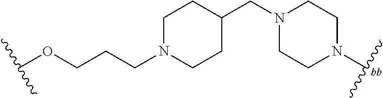 [SMILES: *OCCCN1CCC(CN2CCN(*)CC2)CC1]

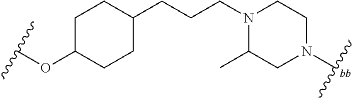 [SMILES: *OC1CCC(CCCN2CCN(*)CC2C)CC1]

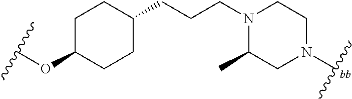 [SMILES: *O[C@H]1CC[C@H](CCCN2CCN(*)C[C@H]2C)CC1]

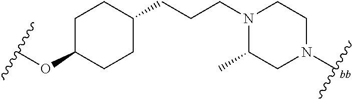 [SMILES: *O[C@H]1CC[C@H](CCCN2CCN(*)C[C@@H]2C)CC1]

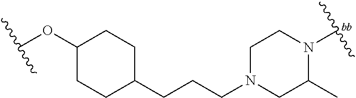 [SMILES: *OC1CCC(CCCN2CCN(*)C(C)C2)CC1]

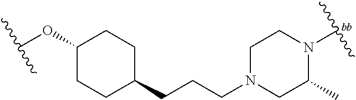 [SMILES: *O[C@H]1CC[C@H](CCCN2CCN(*)[C@H](C)C2)CC1]

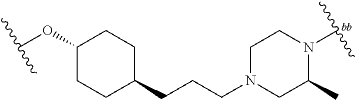 [SMILES: *O[C@H]1CC[C@H](CCCN2CCN(*)[C@@H](C)C2)CC1]

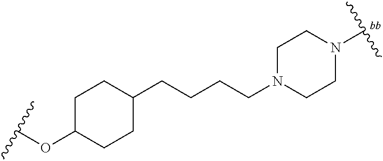 [SMILES: *OC1CCC(CCCCN2CCN(*)CC2)CC1]

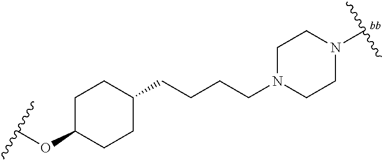 [SMILES: *O[C@H]1CC[C@H](CCCCN2CCN(*)CC2)CC1]

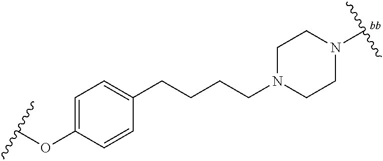 [SMILES: *Oc1ccc(CCCCN2CCN(*)CC2)cc1]

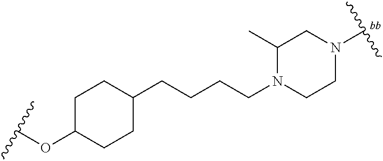 [SMILES: *OC1CCC(CCCCN2CCN(*)CC2C)CC1]

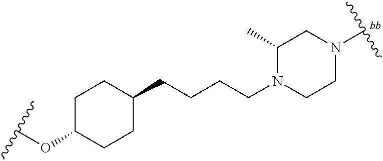 [SMILES: *O[C@H]1CC[C@H](CCCCN2CCN(*)C[C@H]2C)CC1]

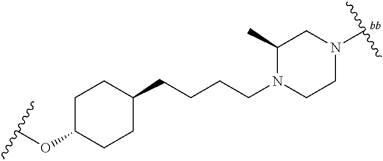 [SMILES: *O[C@H]1CC[C@H](CCCCN2CCN(*)C[C@@H]2C)CC1]

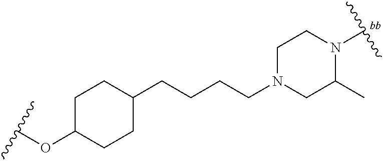 [SMILES: *OC1CCC(CCCCN2CCN(*)C(C)C2)CC1]

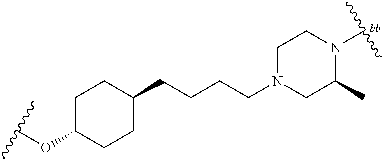 [SMILES: *O[C@H]1CC[C@H](CCCCN2CCN(*)[C@@H](C)C2)CC1]

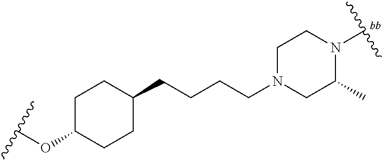 [SMILES: *O[C@H]1CC[C@H](CCCCN2CCN(*)[C@H](C)C2)CC1]

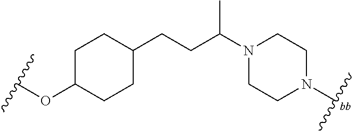 [SMILES: *OC1CCC(CCC(C)N2CCN(*)CC2)CC1]

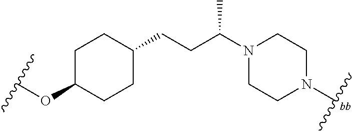 [SMILES: *O[C@H]1CC[C@H](CC[C@H](C)N2CCN(*)CC2)CC1]

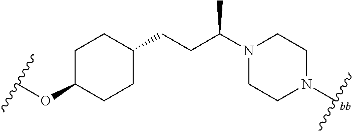 [SMILES: *O[C@H]1CC[C@H](CC[C@@H](C)N2CCN(*)CC2)CC1]

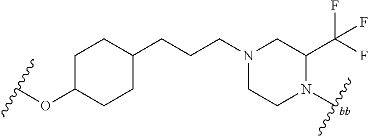 [SMILES: *OC1CCC(CCCN2CCN(*)C(C(F)(F)F)C2)CC1]

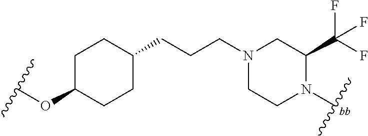 [SMILES: *O[C@H]1CC[C@H](CCCN2CCN(*)[C@H](C(F)(F)F)C2)CC1]

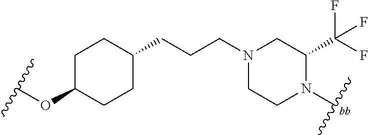 [SMILES: *O[C@H]1CC[C@H](CCCN2CCN(*)[C@@H](C(F)(F)F)C2)CC1]

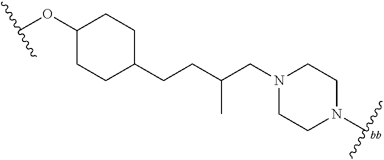 [SMILES: *OC1CCC(CCC(C)CN2CCN(*)CC2)CC1]

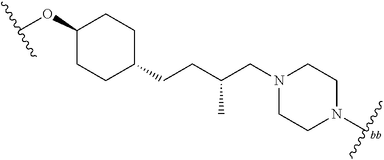 [SMILES: *O[C@H]1CC[C@H](CC[C@@H](C)CN2CCN(*)CC2)CC1]

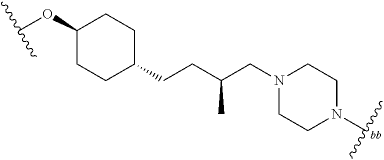 [SMILES: *O[C@H]1CC[C@H](CC[C@H](C)CN2CCN(*)CC2)CC1]

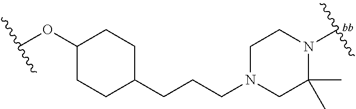 [SMILES: *OC1CCC(CCCN2CCN(*)C(C)(C)C2)CC1]

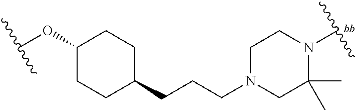 [SMILES: *O[C@H]1CC[C@H](CCCN2CCN(*)C(C)(C)C2)CC1]

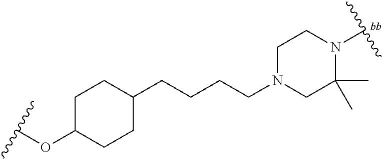 [SMILES: *OC1CCC(CCCCN2CCN(*)C(C)(C)C2)CC1]

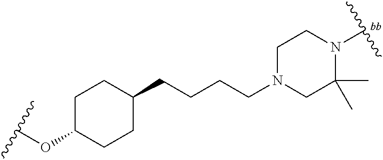 [SMILES: *O[C@H]1CC[C@H](CCCCN2CCN(*)C(C)(C)C2)CC1]

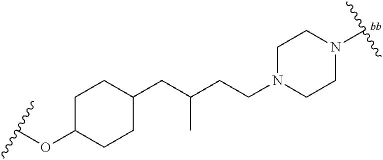 [SMILES: *OC1CCC(CC(C)CCN2CCN(*)CC2)CC1]

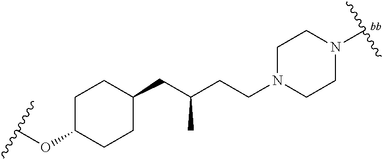 [SMILES: *O[C@H]1CC[C@H](C[C@H](C)CCN2CCN(*)CC2)CC1]

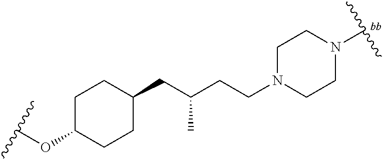 [SMILES: *O[C@H]1CC[C@H](C[C@@H](C)CCN2CCN(*)CC2)CC1]

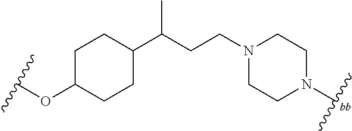 [SMILES: *OC1CCC(C(C)CCN2CCN(*)CC2)CC1]

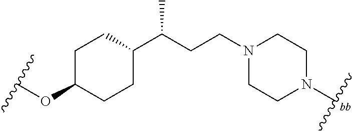 [SMILES: *O[C@H]1CC[C@H]([C@H](C)CCN2CCN(*)CC2)CC1]

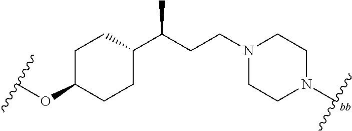 [SMILES: *O[C@H]1CC[C@H]([C@@H](C)CCN2CCN(*)CC2)CC1]

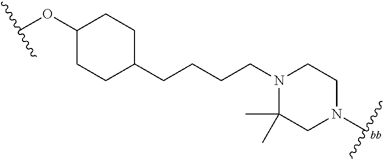 [SMILES: *OC1CCC(CCCCN2CCN(*)CC2(C)C)CC1]

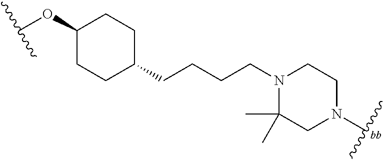 [SMILES: *O[C@H]1CC[C@H](CCCCN2CCN(*)CC2(C)C)CC1]

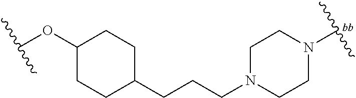 [SMILES: *OC1CCC(CCCN2CCN(*)CC2)CC1]

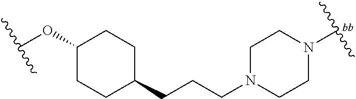 [SMILES: *O[C@H]1CC[C@H](CCCN2CCN(*)CC2)CC1]

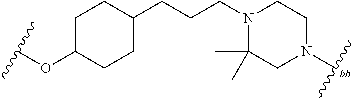 [SMILES: *OC1CCC(CCCN2CCN(*)CC2(C)C)CC1]

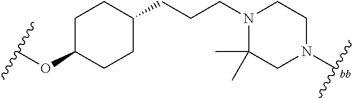 [SMILES: *O[C@H]1CC[C@H](CCCN2CCN(*)CC2(C)C)CC1]

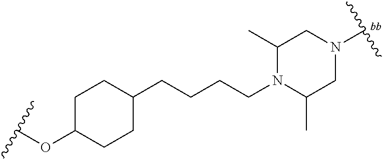 [SMILES: *OC1CCC(CCCCN2C(C)CN(*)CC2C)CC1]

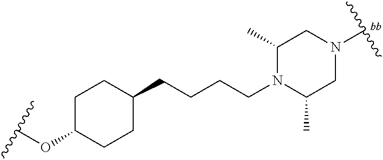 [SMILES: *O[C@H]1CC[C@H](CCCCN2[C@H](C)CN(*)C[C@@H]2C)CC1]

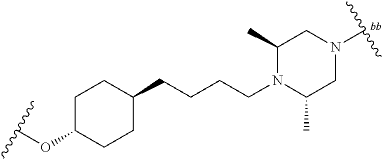 [SMILES: *O[C@H]1CC[C@H](CCCCN2[C@@H](C)CN(*)C[C@@H]2C)CC1]

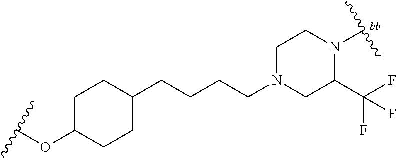 [SMILES: *OC1CCC(CCCCN2CCN(*)C(C(F)(F)F)C2)CC1]

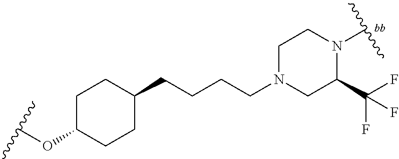 [SMILES: *O[C@H]1CC[C@H](CCCCN2CCN(*)[C@@H](C(F)(F)F)C2)CC1]

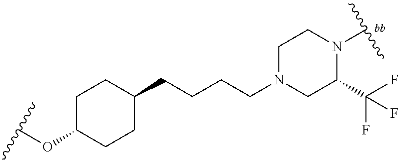 [SMILES: *O[C@H]1CC[C@H](CCCCN2CCN(*)[C@H](C(F)(F)F)C2)CC1]

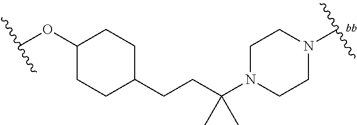 [SMILES: *OC1CCC(CCC(C)(C)N2CCN(*)CC2)CC1]

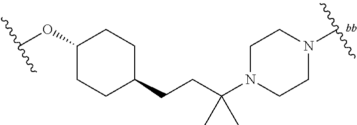 [SMILES: *O[C@H]1CC[C@H](CCC(C)(C)N2CCN(*)CC2)CC1]

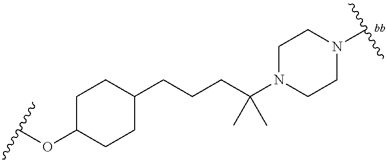 [SMILES: *OC1CCC(CCCC(C)(C)N2CCN(*)CC2)CC1]

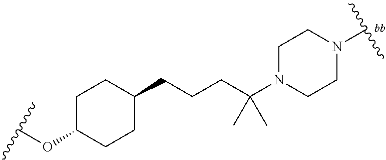 [SMILES: *O[C@H]1CC[C@H](CCCC(C)(C)N2CCN(*)CC2)CC1]

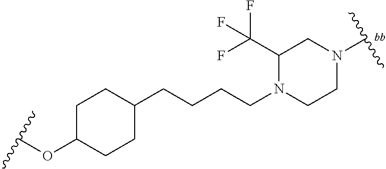 [SMILES: *OC1CCC(CCCCN2CCN(*)CC2C(F)(F)F)CC1]

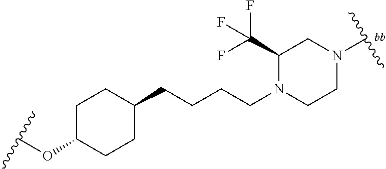 [SMILES: *O[C@H]1CC[C@H](CCCCN2CCN(*)C[C@@H]2C(F)(F)F)CC1]

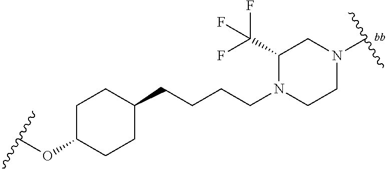 [SMILES: *O[C@H]1CC[C@H](CCCCN2CCN(*)C[C@H]2C(F)(F)F)CC1]

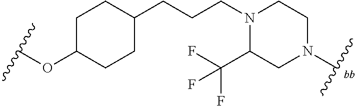 [SMILES: *OC1CCC(CCCN2CCN(*)CC2C(F)(F)F)CC1]

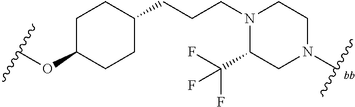 [SMILES: *O[C@H]1CC[C@H](CCCN2CCN(*)C[C@@H]2C(F)(F)F)CC1]

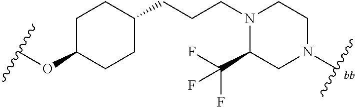 [SMILES: *O[C@H]1CC[C@H](CCCN2CCN(*)C[C@H]2C(F)(F)F)CC1]

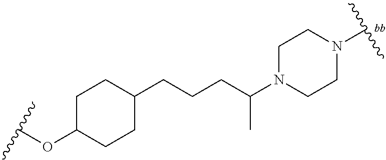 [SMILES: *OC1CCC(CCCC(C)N2CCN(*)CC2)CC1]

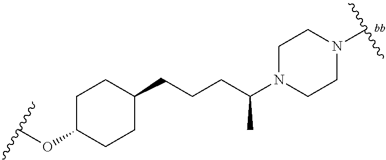 [SMILES: *O[C@H]1CC[C@H](CCC[C@H](C)N2CCN(*)CC2)CC1]

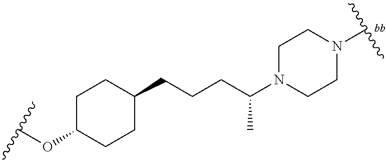 [SMILES: *O[C@H]1CC[C@H](CCC[C@@H](C)N2CCN(*)CC2)CC1]

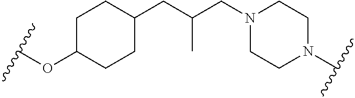 [SMILES: *OC1CCC(CC(C)CN2CCN(*)CC2)CC1]

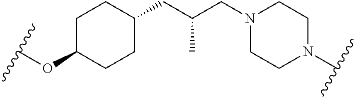 [SMILES: *O[C@H]1CC[C@H](C[C@@H](C)CN2CCN(*)CC2)CC1]

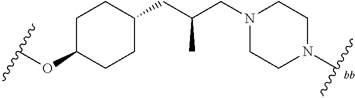 [SMILES: *O[C@H]1CC[C@H](C[C@H](C)CN2CCN(*)CC2)CC1]

Examples when L is (L-4)

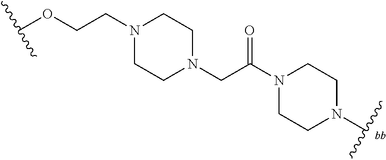 [SMILES: *OCCN1CCN(CC(=O)N2CCN(*)CC2)CC1]

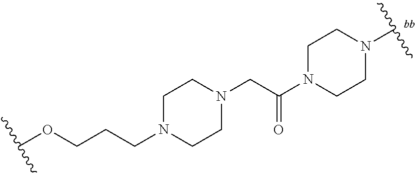 [SMILES: *OCCCN1CCN(CC(=O)N2CCN(*)CC2)CC1]

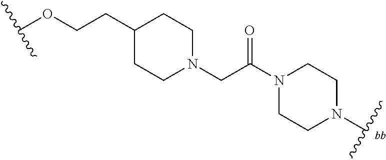 [SMILES: *OCCC1CCN(CC(=O)N2CCN(*)CC2)CC1]

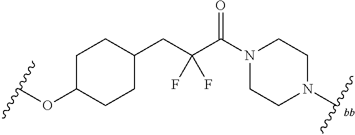 [SMILES: *OC1CCC(CC(F)(F)C(=O)N2CCN(*)CC2)CC1]

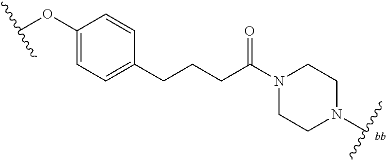 [SMILES: *Oc1ccc(CCCC(=O)N2CCN(*)CC2)cc1]

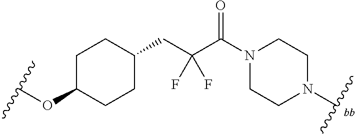 [SMILES: *O[C@H]1CC[C@H](CC(F)(F)C(=O)N2CCN(*)CC2)CC1]

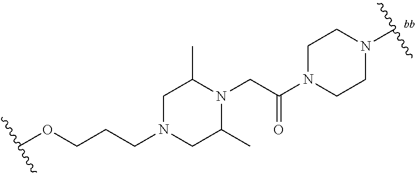 [SMILES: *OCCCN1CC(C)N(CC(=O)N2CCN(*)CC2)C(C)C1]

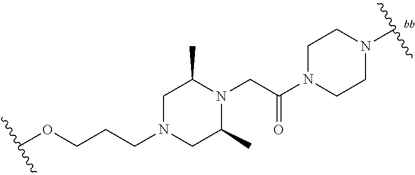 [SMILES: *OCCCN1C[C@@H](C)N(CC(=O)N2CCN(*)CC2)[C@@H](C)C1]

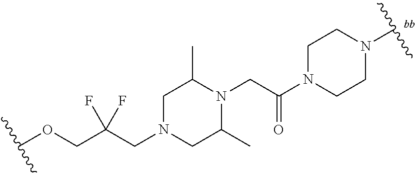 [SMILES: *OCC(F)(F)CN1CC(C)N(CC(=O)N2CCN(*)CC2)C(C)C1]

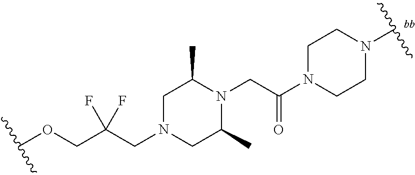 [SMILES: *OCC(F)(F)CN1C[C@@H](C)N(CC(=O)N2CCN(*)CC2)[C@@H](C)C1]

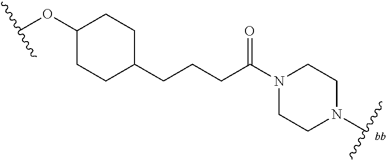 [SMILES: *OC1CCC(CCCC(=O)N2CCN(*)CC2)CC1]

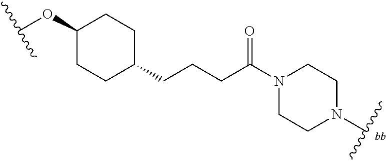 [SMILES: *O[C@H]1CC[C@H](CCCC(=O)N2CCN(*)CC2)CC1]

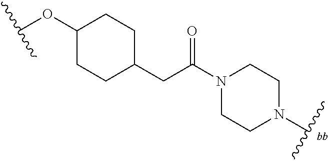 [SMILES: *OC1CCC(CC(=O)N2CCN(*)CC2)CC1]

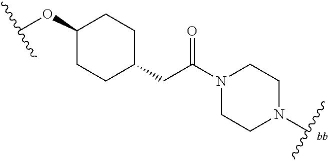 [SMILES: *O[C@H]1CC[C@H](CC(=O)N2CCN(*)CC2)CC1]

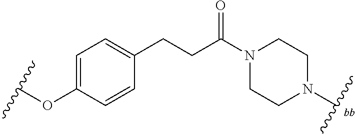 [SMILES: *Oc1ccc(CCC(=O)N2CCN(*)CC2)cc1]

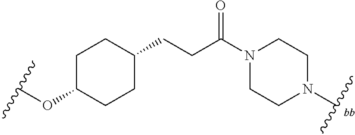 [SMILES: *O[C@H]1CC[C@@H](CCC(=O)N2CCN(*)CC2)CC1]

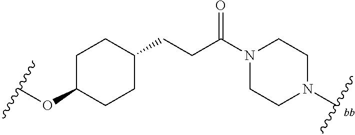 [SMILES: *O[C@H]1CC[C@H](CCC(=O)N2CCN(*)CC2)CC1]

Examples when L is (L-3a)

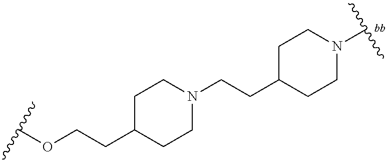 [SMILES: *OCCC1CCN(CCC2CCN(*)CC2)CC1]

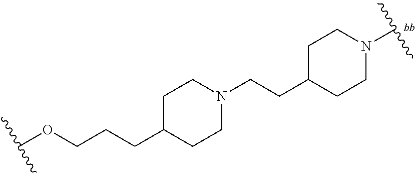 [SMILES: *OCCCC1CCN(CCC2CCN(*)CC2)CC1]

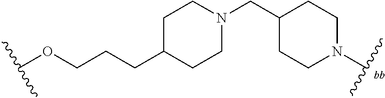 [SMILES: *OCCCC1CCN(CC2CCN(*)CC2)CC1]

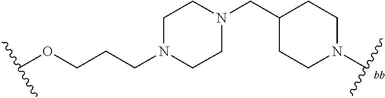 [SMILES: *OCCCN1CCN(CC2CCN(*)CC2)CC1]

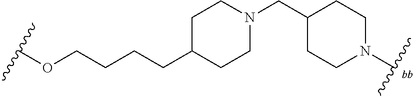 [SMILES: *OCCCCC1CCN(CC2CCN(*)CC2)CC1]

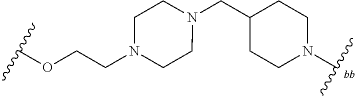 [SMILES: *OCCN1CCN(CC2CCN(*)CC2)CC1]

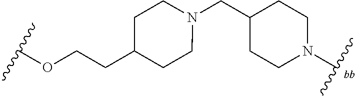 [SMILES: *OCCC1CCN(CC2CCN(*)CC2)CC1]

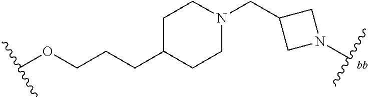 [SMILES: *OCCCC1CCN(CC2CN(*)C2)CC1]

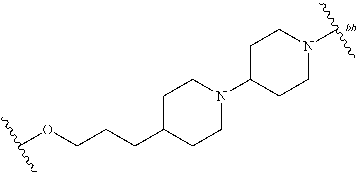 [SMILES: *OCCCC1CCN(C2CCN(*)CC2)CC1]

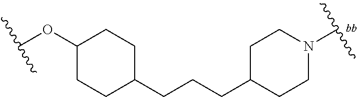 [SMILES: *OC1CCC(CCCC2CCN(*)CC2)CC1]

Examples when L is (L-3b)

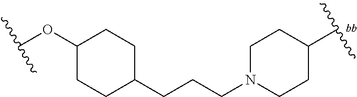 [SMILES: *OC1CCC(CCCN2CCC(*)CC2)CC1]

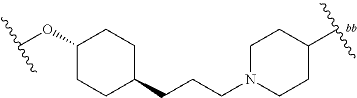 [SMILES: *O[C@H]1CC[C@H](CCCN2CCC(*)CC2)CC1]

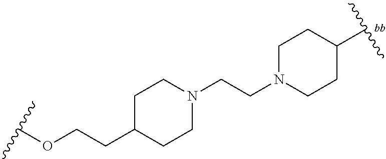 [SMILES: *OCCC1CCN(CCN2CCC(*)CC2)CC1]

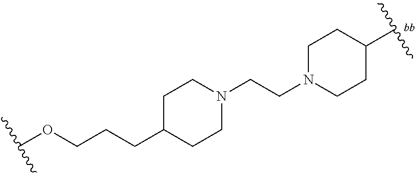 [SMILES: *OCCCC1CCN(CCN2CCC(*)CC2)CC1]

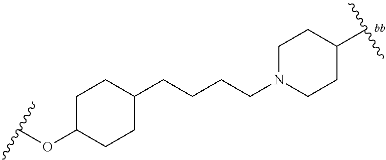 [SMILES: *OC1CCC(CCCCN2CCC(*)CC2)CC1]

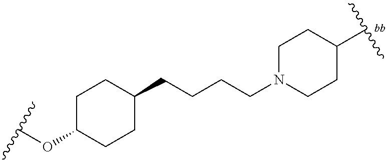 [SMILES: *O[C@H]1CC[C@H](CCCCN2CCC(*)CC2)CC1]

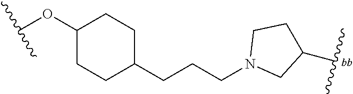 [SMILES: *OC1CCC(CCCN2CCC(*)C2)CC1]

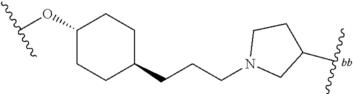 [SMILES: *O[C@H]1CC[C@H](CCCN2CCC(*)C2)CC1]

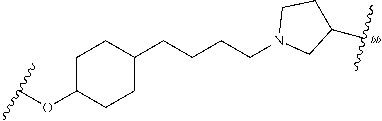 [SMILES: *OC1CCC(CCCCN2CCC(*)C2)CC1]

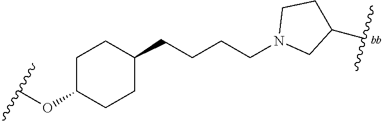 [SMILES: *O[C@H]1CC[C@H](CCCCN2CCC(*)C2)CC1]

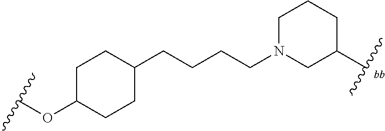 [SMILES: *OC1CCC(CCCCN2CCCC(*)C2)CC1]

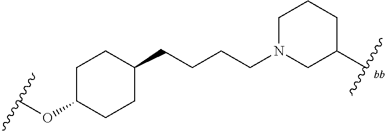 [SMILES: *O[C@H]1CC[C@H](CCCCN2CCCC(*)C2)CC1]

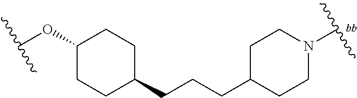 [SMILES: *O[C@H]1CC[C@H](CCCC2CCN(*)CC2)CC1]

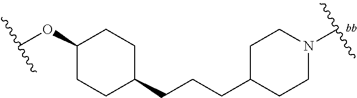 [SMILES: *O[C@H]1CC[C@@H](CCCC2CCN(*)CC2)CC1]

Examples when L is (L-3c)

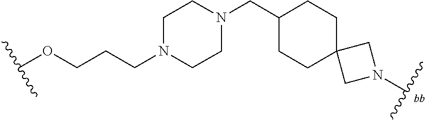 [SMILES: *OCCCN1CCN(CC2CCC3(CC2)CN(*)C3)CC1]

Examples when L is (L-3d)

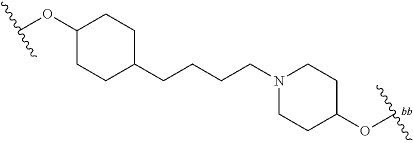 [SMILES: *OC1CCC(CCCCN2CCC(O*)CC2)CC1]

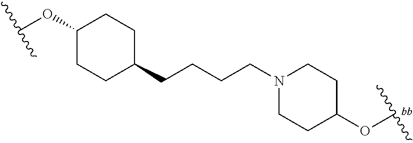 [SMILES: *OC1CCN(CCCC[C@H]2CC[C@H](O*)CC2)CC1]

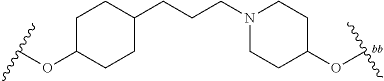 [SMILES: *OC1CCC(CCCN2CCC(O*)CC2)CC1]

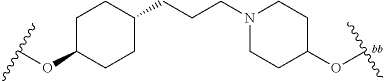 [SMILES: *OC1CCN(CCC[C@H]2CC[C@H](O*)CC2)CC1]

Examples when L is (L-4a)

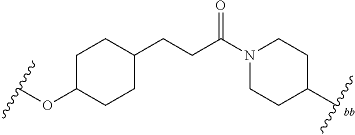 [SMILES: *OC1CCC(CCC(=O)N2CCC(*)CC2)CC1]

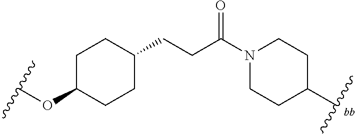 [SMILES: *O[C@H]1CC[C@H](CCC(=O)N2CCC(*)CC2)CC1]

Examples when L is (L-5)

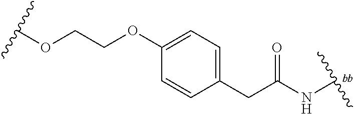

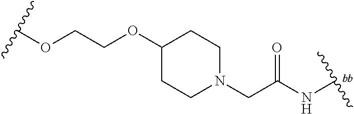

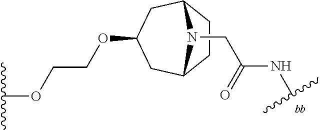 [SMILES: *NC(=O)CN1[C@@H]2CC[C@H]1C[C@H](OCCO*)C2]

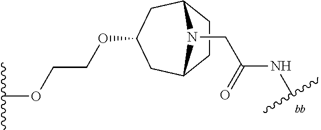 [SMILES: *NC(=O)CN1[C@@H]2CC[C@H]1C[C@@H](OCCO*)C2]

Examples when L is (L-5a)

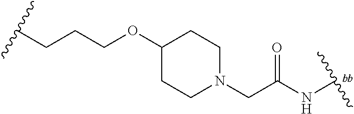

Examples when L is (L-6)

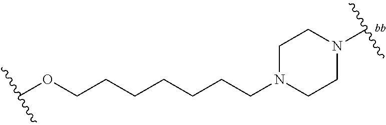 [SMILES: *OCCCCCCCN1CCN(*)CC1]

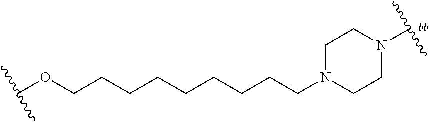 [SMILES: *OCCCCCCCCCN1CCN(*)CC1]

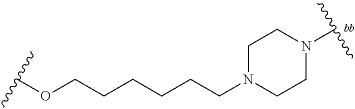 [SMILES: *OCCCCCCN1CCN(*)CC1]

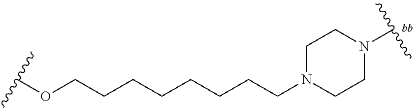 [SMILES: *OCCCCCCCCN1CCN(*)CC1]

In some embodiments of Formula (I), L is selected from the group consisting of the moieties depicted in Table (L); and

Ring A is

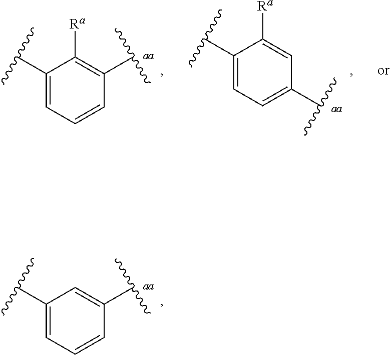

wherein aa represents the point of attachment to L, wherein the R^{a} present on Ring A is methyl or CF₃; R¹ is C(O)OH or C(O)OC₁₋₆ alkyl.

In some embodiments of Formula (I), L is selected from the group consisting of the moieties depicted in Table (L);
R^{aN} is C₁₋₃ alkyl (e.g., methyl); c1 is 0 or 1; and R^{a2} is selected from the group consisting of halo (e.g., —F) and C₁₋₃ alkyl optionally substituted with 1-3 F.

In some embodiments of Formula (I), L is selected from the group consisting of the moieties depicted in Table (L);
Ring A is

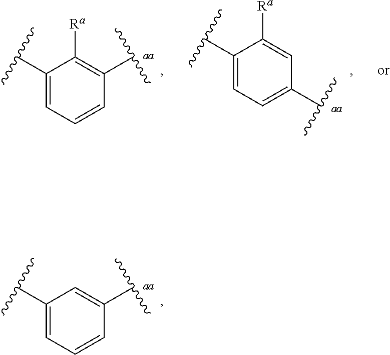

wherein aa represents the point of attachment to L, wherein the R^{a} present on Ring A is methyl or CF₃; R¹ is C(O)OH or C(O)OC₁₋₆ alkyl; and
Ring C is

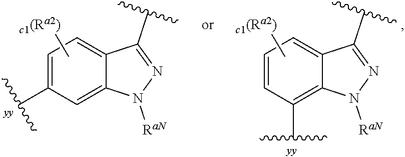

wherein R^{aN} is C₁₋₃ alkyl (e.g., methyl); c1 is 0 or 1; and R^{a2} is selected from the group consisting of halo (e.g., —F) and C₁₋₃ alkyl optionally substituted with 1-3 F.

In some embodiments, L is (L-1); and the compounds of Formula (I) are compounds of Formula (I-A):

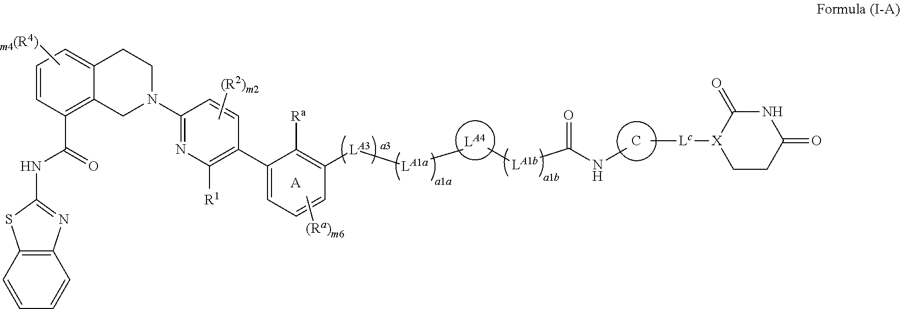

or pharmaceutically acceptable salts thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 1 to 5 (e.g., from 2 to 5);
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

In some embodiments, L is (L-1); and the compounds of Formula (I) are compounds of Formula (I-B):

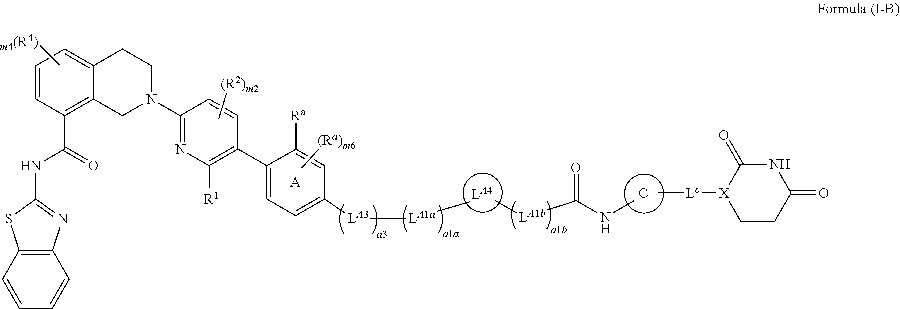

or pharmaceutically acceptable salts thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 1 to 5 (e.g., from 2 to 5);
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

In some embodiments of Formula (I-A) or (I-B), a3 is 1. In some embodiments, L^{A3} is —O—. In some embodiments of Formula (I-A) or (I-B), a3 is 1; and L^{A3} is —O—.

In some embodiments of Formula (I-A) or (I-B), a3 is 0.

In some embodiments, L is (L-2); and the compounds of Formula (I) are compounds of Formula (I-C):

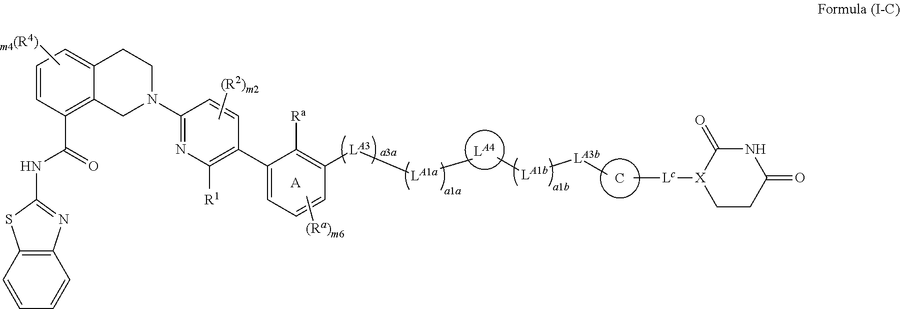

or pharmaceutically acceptable salts thereof, wherein:
m6 is 0 or 1;
a3a is 0 or 1;
L^{A3a} and L^{A3b} are independently selected from the group consisting of: —O—, —N(H)—, and —N(C₁₋₃ alkyl)-;
a1a and a1b are independently integers from 0 to 5, provided that a1a+a1b is from 2 to 7;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

In some embodiments, L is (L-2); and the compounds of Formula (I) are compounds of Formula (I-D):

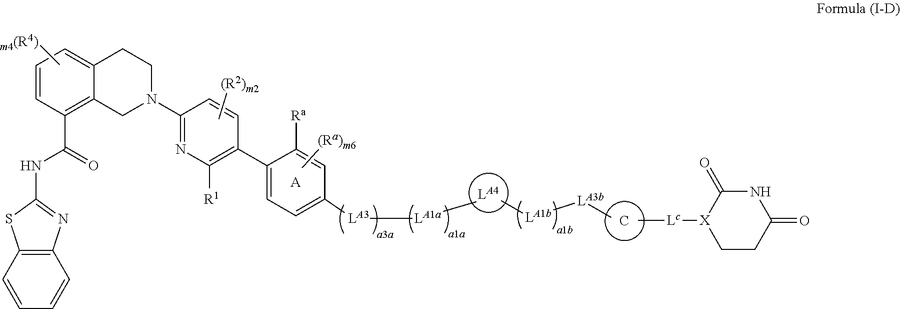

or pharmaceutically acceptable salts thereof, wherein:
m6 is 0 or 1;
a3a is 0 or 1;
L^{A3a} and L^{A3b} are independently selected from the group consisting of: —O—, —N(H)—, and —N(C₁₋₃ alkyl)-;
a1a and a1b are independently integers from 0 to 5, provided that a1a+a1b is from 2 to 7;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

In some embodiments of Formula (I-C) or (I-D), L^{A3b} is —N(H)— or —N(C₁₋₃ alkyl)-.

In some embodiments of Formula (I-C) or (I-D), a3a is 1. In some embodiments, L^{A3a} is —O—.

In some embodiments of Formula (I-C) or (I-D), a3a is 0.

In some embodiments, L is (L-3); and the compounds of Formula (I) are compounds of Formula (I-E):

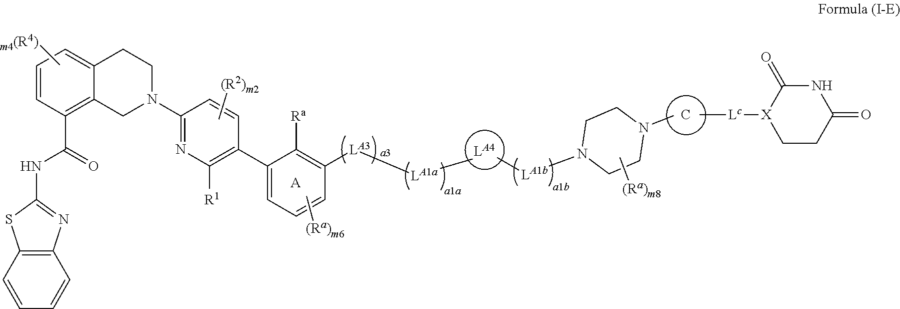

or pharmaceutically acceptable salts thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
m8 is 0, 1, or 2.

In some embodiments, L is (L-3a); and the compounds of Formula (I) are compounds of Formula (I-Ea):

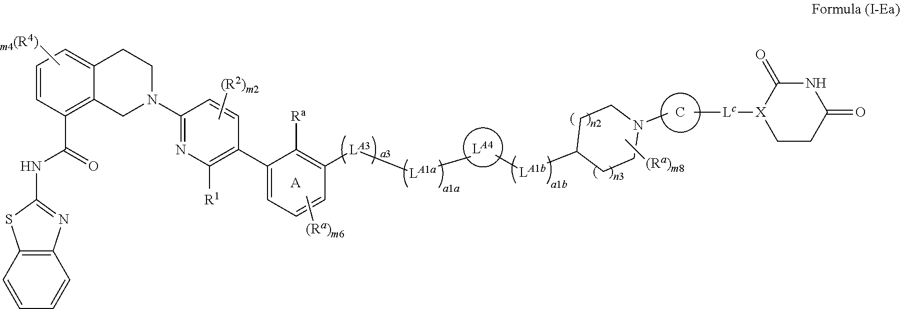

or pharmaceutically acceptable salts thereof, wherein:
m6 is 0 or 1;
a3 is 0 or 1;
L^{A3} is selected from the group consisting of: —O—, —N(H)—, and —N(C₁₋₃ alkyl)-; a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
n2 and n3 are independently 0, 1, or 2; and
m8 is 0, 1, or 2.

In some embodiments, L is (L-3b); and the compounds of Formula (I) are compounds of Formula (I-Eb):

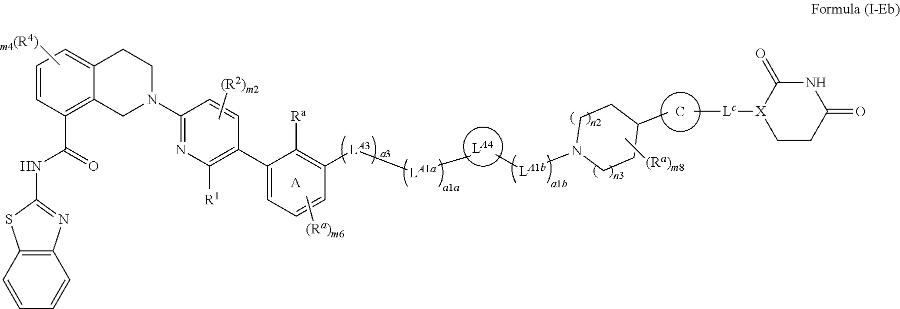

or pharmaceutically acceptable salts thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
n2 and n3 are independently 0, 1, or 2; and
m8 is 0, 1, or 2.

In some embodiments, L is (L-4); and the compounds of Formula (I) are compounds of Formula (I-F):

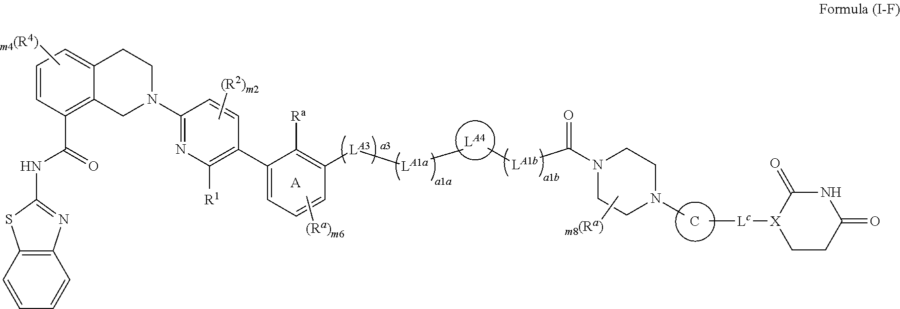

or pharmaceutically acceptable salts thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
m8 is 0, 1, or 2.

In some embodiments of Formula (I-E), (I-Ea), (I-Eb), or (I-F), m8 is 0. In some embodiments of Formula (I-E), (I-Ea), (I-Eb), or (I-F), m8 is 1. In some embodiments of Formula (I-E), (I-Ea), (I-Eb), or (I-F), m8 is 2.

In some embodiments of Formula (I-Ea) or (I-Eb), n2 is 1; and n3 is 1. In some embodiments of Formula (I-Ea) or (I-Eb), n2 is 0; and n3 is 1.

In some embodiments, L is (L-3) or (L-4); and the compounds of Formula (I) are compounds of Formula (I-E) or (I-F), or pharmaceutically acceptable salts thereof. In some embodiments, the compounds are compounds of Formula (I-E), or pharmaceutically acceptable salts thereof. In some embodiments, the compounds are compounds of Formula (I-F), or pharmaceutically acceptable salts thereof. In some embodiments, the compounds are compounds of Formula (I-Ea), or pharmaceutically acceptable salts thereof. In some embodiments, the compounds are compounds of Formula (I-Eb), or pharmaceutically acceptable salts thereof.

In some embodiments, L is (L-3); and the compounds of Formula (I) are compounds of Formula (I-G):

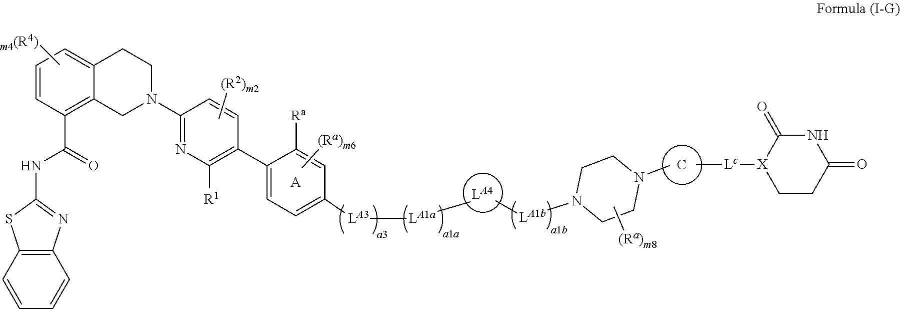

or pharmaceutically acceptable salts thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
m8 is 0, 1, or 2.

In some embodiments, L is (L-3a); and the compounds of Formula (I) are compounds of Formula (I-Ga):

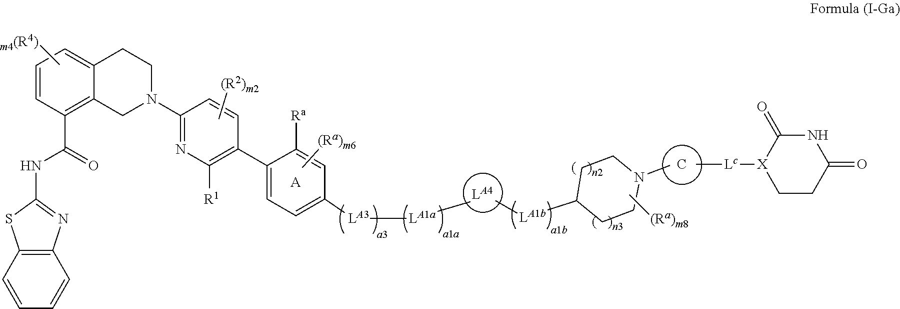

or pharmaceutically acceptable salts thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
n2 and n3 are independently 0, 1, or 2; and
m8 is 0, 1, or 2.

In some embodiments, L is (L-4); and the compounds of Formula (I) are compounds of Formula (I-H):

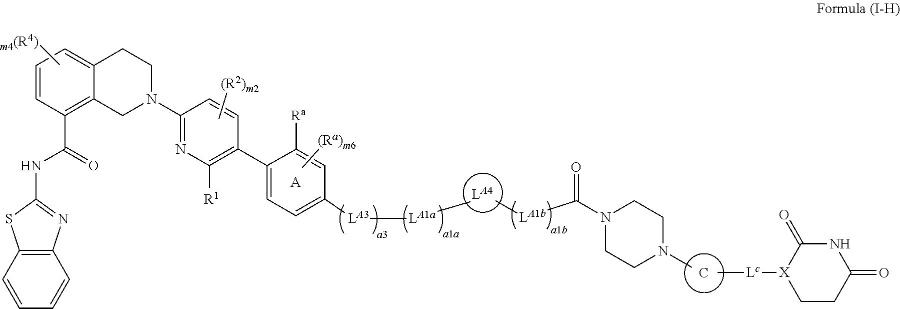

or pharmaceutically acceptable salts thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
m8 is 0, 1, or 2.

In some embodiments, L is (L-3) or (L-4); and the compounds of Formula (I) are compounds of Formula (I-G) or (I-H), or pharmaceutically acceptable salts thereof. In some embodiments, the compounds are compounds of Formula (I-G), or pharmaceutically acceptable salts thereof. In some embodiments, the compounds are compounds of Formula (I-H), or pharmaceutically acceptable salts thereof. In some embodiments, the compounds are compounds of Formula (I-Ga), or pharmaceutically acceptable salts thereof.

In some embodiments of Formula (I-E), (I-Ea), (I-Eb), (I-F), (I-G), (I-Ga), or (I-H), a3 is 1. In some embodiments, L^{A3} is —O—. In some embodiments of Formula (I-E), (I-Ea), (I-Eb), (I-F), (I-G), (I-Ga), or (I-H), a3 is 1; and L^{A3} is —O—.

In some embodiments of Formula (I-E), (I-Ea), (I-Eb), (I-F), (I-G), (I-Ga), or (I-H), a3 is 0.

In some embodiments of Formula (I-A), (I-B), (I-C), (I-D), (I-E), (I-Ea), (I-Eb) (I-F), (I-G), (I-Ga), or (I-H), a1a+a1b is 3.

In some embodiments of Formula (I-A), (I-B), (I-C), (I-D), (I-E), (I-Ea), (I-Eb) (I-F), (I-G), (I-Ga), or (I-H), a1a+a1b is 4.

In some embodiments of Formula (I-A), (I-B), (I-C), (I-D), (I-E), (I-Ea), (I-Eb) (I-F), (I-G), (I-Ga), or (I-H), a1a+a1b is 2 or 5. For example, a1a+a1b can be 5.

In some embodiments of Formula (I-A), (I-B), (I-C), (I-D), (I-E), (I-Ea), (I-Eb) (I-F), (I-G), (I-Ga), or (I-H), each occurrence of L^{A1a} and L^{A1b} is —CH₂—.

In some embodiments of Formula (I-A), (I-B), (I-C), (I-D), (I-E), (I-Ea), (I-Eb) (I-F), (I-G), (I-Ga), or (I-H), one occurrence of L^{A1a} is —CHR^{L}— or —C(R^{L})₂—; each remaining occurrence of L^{A1a} is —CH₂—; and each occurrence of L^{A1b} is —CH₂—.

In some embodiments of Formula (I-A), (I-B), (I-C), (I-D), (I-E), (I-Ea), (I-Eb) (I-F), (I-G), (I-Ga), or (I-H), one occurrence of L^{A1b} is —CHR^{L}— or —C(R^{L})₂—; each remaining occurrence of L^{A1b} is —CH₂—; and each occurrence of L^{A1a} is —CH₂—.

In some embodiments of Formula (I-A), (I-B), (I-C), (I-D), (I-E), (I-Ea), (I-Eb) (I-F), (I-G), (I-Ga), or (I-H), L^{A4} is 4-10 membered (e.g., monocyclic or bicyclic) heterocyclylene optionally substituted with 1-3 R^{a}. For example, L^{A4} can be selected from the group consisting of:

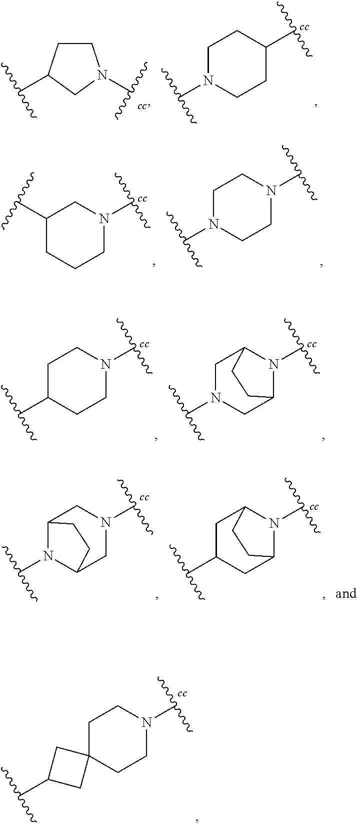

each optionally substituted with 1-3 R^{a} at one or more ring carbon atoms, wherein cc represents the point of attachment to L^{A1b}.

In some embodiments of Formula (I-A), (I-B), (I-C), (I-D), (I-E), (I-Ea), (I-Eb) (I-F), (I-G), (I-Ga), or (I-H), each R^{a} is independently selected from the group consisting of: halo, —OH, C₁₋₃ alkoxy, C₁₋₃ haloalkoxy, and C₁₋₃ alkyl optionally substituted with 1-3 R^{c}.

In some embodiments of Formula (I-A), (I-B), (I-C), (I-D), (I-E), (I-Ea), (I-Eb) (I-F), (I-G), (I-Ga), or (I-H), each R^{a} present on L^{A4} is independently a C₁₋₃ alkyl optionally substituted with 1-3 F.

In some embodiments of Formula (I-A), (I-B), (I-C), (I-D), (I-E), (I-Ea), (I-Eb) (I-F), (I-G), (I-Ga), or (I-H), L^{A4} is C₃₋₁₀ cycloalkylene optionally substituted with 1-3 R^{a}. For example, L^{A4} can be 1,4-cyclohexylene optionally substituted with 1-3 R^{a}.

In some embodiments of Formula (I-A), (I-B), (I-C), (I-D), (I-E), (I-Ea), (I-Eb) (I-F), (I-G), (I-Ga), or (I-H), L^{A4} is phenylene or 5-6 membered heteroarylene, each of which is optionally substituted with 1-3 R^{a}. For example, L^{A4} can be 1,4-phenylene optionally substituted with 1-3 R^{a}. For example, L^{A4} can be 1,2-phenylene or 1,3-phenylene, each of which is optionally substituted with 1-3 R^{a}.

In some embodiments of Formula (I-A), (I-B), (I-C), (I-D), (I-E), (I-Ea), (I-Eb) (I-F), (I-G), (I-Ga), or (I-H), m6 is 0.

In some embodiments of Formula (I-A), (I-B), (I-C), (I-D), (I-E), (I-Ea), (I-Eb) (I-F), (I-G), (I-Ga), or (I-H), the R^{a} present on Ring A is C₁₋₃ alkyl optionally substituted with 1-3-F.

In some embodiments of Formula (I-A), (I-B), (I-C), (I-D), (I-E), (I-Ea), (I-Eb) (I-F), (I-G), (I-Ga), or (I-H), R¹ is C(O)OH. In some embodiments of Formula (I-A), (I-B), (I-C), (I-D), (I-E), (I-Ea), (I-Eb) (I-F), (I-G), (I-Ga), or (I-H), R¹ is C(O)OC₁₋₆ alkyl.

In some embodiments, L is (L-7) wherein a3a is 0; and the compounds of Formula (I) are compounds of Formula (II-A):

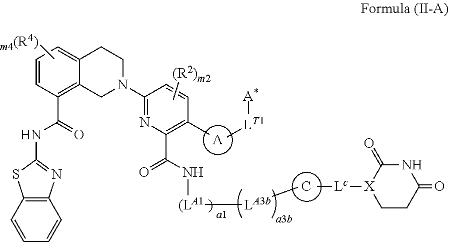

or pharmaceutically acceptable salts thereof, wherein:
Ring A is phenylene or 5-6 membered heteroarylene, each of which is optionally substituted with 1-3 R^{a};
L^{T1} is a bond or CH₂;
A* is H or C₃₋₁₅ cycloalkyl optionally substituted with 1-3 R^{a};
L^{A3b} is selected from the group consisting of: C(═O), —O—, N(H)—, and —N(C₁₋₃ alkyl)-;
a3b is 1 or 2; and
a1 is an integer from 1 to 15.

In some embodiments of Formula (II-A), Ring A is 5-membered heteroarylene optionally substituted with 1-2 R^{a}. In some embodiments of Formula (II-A), Ring A is selected from the group consisting of:

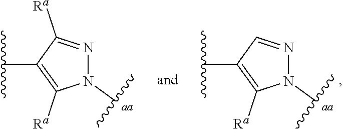

wherein aa represents the point of attachment to L^{T1}.

In some embodiments of Formula (IT-A), L^{T1} is CH₂.

In some embodiments of Formula (IT-A), A* is H.

In some embodiments of Formula (II-A), A* is C₃₋₁₅ cycloalkyl optionally substituted with 1-3 R^{a}. For example, A* can be adamantyl optionally substituted with 1-3 R^{a}.

In some embodiments of Formula (II-A), a1 is an integer from 1 to 3. In some embodiments of Formula (II-A), a1 is an integer from 4 to 6. In some embodiments of Formula (II-A), a1 is an integer from 7 to 9. In some embodiments of Formula (II-A), a1 is an integer from 10 to 15.

In some embodiments of Formula (II-A), -(L^{A3b})_{a3b}- is

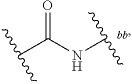

wherein bb represents the point of attachment to Ring C.

In some embodiments of Formula (I-A), (I-B), (I-C), (I-D), (I-E), (I-Ea), (I-Eb), (I-F), (I-G), (I-Ga), (I-H), or (II-A), m2 is 0. In some embodiments of Formula (I-A), (I-B), (I-C), (I-D), (I-E), (I-Ea), (I-Eb), (I-F), (I-G), (I-Ga), (I-H), or (II-A), m4 is 0.

In some embodiments of Formula (I-A), (I-B), (I-C), (I-D), (I-E), (I-Ea), (I-Eb), (I-F), (I-G), (I-Ga), (I-H), or (II-A), Ring C is

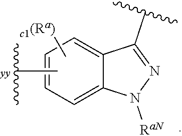

In some embodiments of Formula (I-A), (I-B), (I-C), (I-D), (I-E), (I-Ea), (I-Eb), (I-F), (I-G), (I-Ga), (I-H), or (II-A), Ring C is

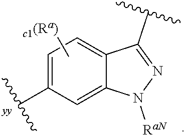

In some embodiments of Formula (I-A), (I-B), (I-C), (I-D), (I-E), (I-Ea), (I-Eb), (I-F), (I-G), (I-Ga), (I-H), or (II-A), Ring C is

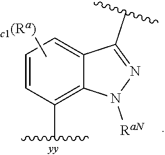

In some embodiments of Formula (I-A), (I-B), (I-C), (I-D), (I-E), (I-Ea), (I-Eb), (I-F), (I-G), (I-Ga), (I-H), or (II-A), R^{aN} is C₁₋₃ alkyl. For example, R^{aN} can be methyl.

In some embodiments of Formula (I-A), (I-B), (I-C), (I-D), (I-E), (I-Ea), (I-Eb), (I-F), (I-G), (I-Ga), (I-H), or (II-A), Ring C is

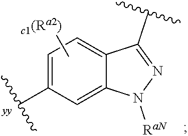

R^{aN} is C₁₋₃ alkyl (e.g., methyl); c1 is 0 or 1; and R^{a2} is selected from the group consisting of: halo (e.g., —F) and C₁₋₃ alkyl optionally substituted with 1-3-F. For example, Ring C can be

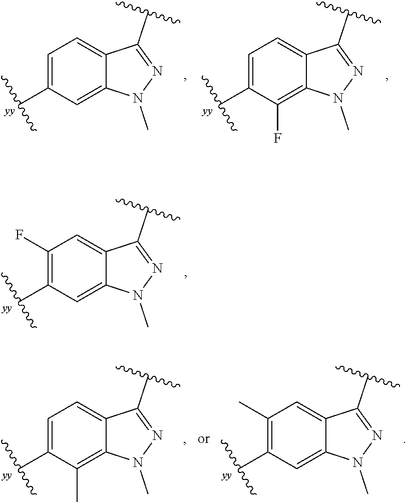 [SMILES: *c1ccc2c(*)nn(C)c2c1 ; *c1ccc2c(*)nn(C)c2c1F ; *c1cc2c(cc1F)c(*)nn2C ; *c1ccc2c(*)nn(C)c2c1C ; *c1cc2c(cc1C)c(*)nn2C]

In some embodiments of Formula (I-A), (I-B), (I-C), (I-D), (I-E), (I-Ea), (I-Eb), (I-F), (I-G), (I-Ga), (I-H), or (II-A), Ring C is

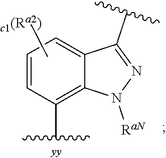

R^{aN} is C₁₋₃ alkyl (e.g., methyl); c1 is 0 or 1; and R^{a}z is selected from the group consisting of halo (e.g., —F), and C₁₋₃ alkyl optionally substituted with 1-3-F. For example, Ring C can be

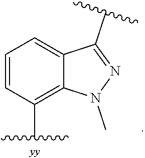 [SMILES: *c1nn(C)c2c(*)cccc12]

In some embodiments of Formula (I-A), (I-B), (I-C), (I-D), (I-E), (I-Ea), (I-Eb), (I-F), (I-G), (I-Ga), (I-H), or (II-A), Ring C is

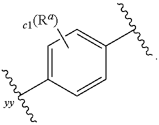

In some embodiments of Formula (I-A), (I-B), (I-C), (I-D), (I-E), (I-Ea), (I-Eb), (I-F), (I-G), (I-Ga), (I-H), or (II-A), Ring C is

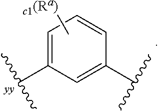

In some embodiments of Formula (I-A), (I-B), (I-C), (I-D), (I-E), (I-Ea), (I-Eb), (I-F), (I-G), (I-Ga), (I-H), or (II-A), c1 is 0.

In some embodiments of Formula (I-A), (I-B), (I-C), (I-D), (I-E), (I-Ea), (I-Eb), (I-F), (I-G), (I-Ga), (I-H), or (II-A), L^{C} is a bond. In some embodiments of Formula (I-A), (I-B), (I-C), (I-D), (I-E), (I-Ea), (I-Eb), (I-F), (I-G), (I-Ga), (I-H), or (II-A), L^{C} is —NH—.

In some embodiments of Formula (I-A), (I-B), (I-C), (I-D), (I-E), (I-Ea), (I-Eb), (I-F), (I-G), (I-Ga), (I-H), or (II-A), X is CH.

In some embodiments of Formula (I-A), (I-B), (I-C), (I-D), (I-E), (I-Ea), (I-Eb), (I-F), (I-G), (I-Ga), (I-H), or (II-A), the

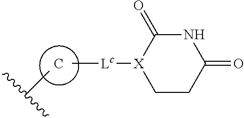

moiety is

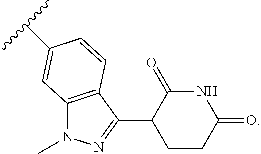 [SMILES: *c1ccc2c(C3CCC(=O)NC3=O)nn(C)c2c1]

In some embodiments of Formula (I-A), (I-B), (I-C), (I-D), (I-E), (I-Ea), (I-Eb), (I-F), (I-G), (I-Ga), (I-H), or (II-A), the

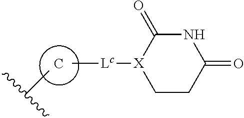

moiety is

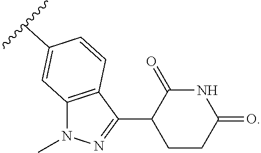 [SMILES: *c1ccc2c(C3CCC(=O)NC3=O)nn(C)c2c1]

In some embodiments of Formula (I-A), (I-B), (I-C), (I-D), (I-E), (I-Ea), (I-Eb), (I-F), (I-G), (I-Ga), (I-H), or (II-A), the

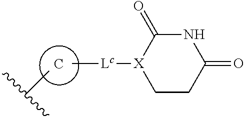

moiety is

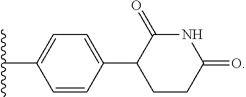 [SMILES: *c1ccc(C2CCC(=O)NC2=O)cc1]

In some embodiments of Formula (I-A), (I-B), (I-C), (I-D), (I-E), (I-Ea), (I-Eb), (I-F), (I-G), (I-Ga), (I-H), or (II-A), the

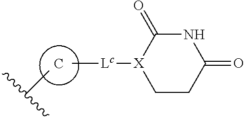

moiety is

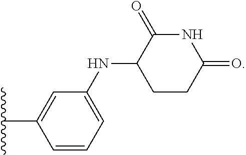 [SMILES: *c1cccc(NC2CCC(=O)NC2=O)c1]

In some embodiments, the compounds of Formula (I-A) are compounds of Formula (I-A-1):

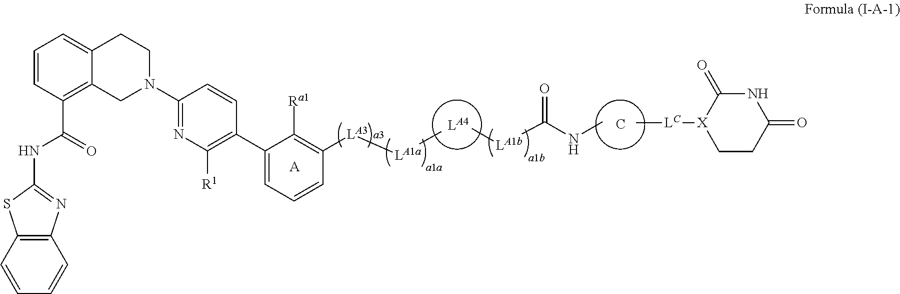

or pharmaceutically acceptable salts thereof, wherein:
R¹ is C(O)OH or C(O)OC₁₋₆ alkyl;
R^{a1} is C₁₋₃ alkyl optionally substituted with 1-3 F;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 1 to 5 (e.g., from 2 to 5);
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

In some embodiments, the compounds of Formula (I-A) are compounds of Formula (I-A-2):

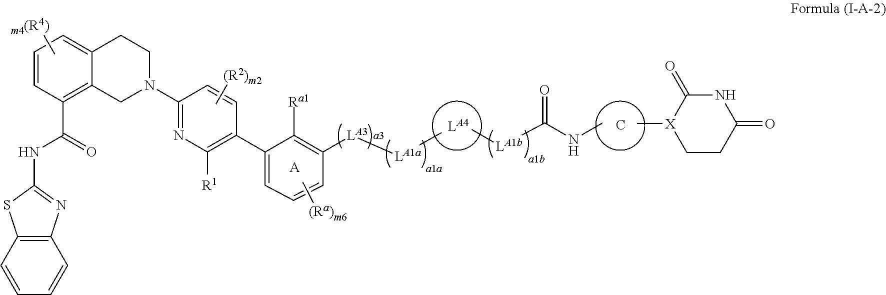

or pharmaceutically acceptable salts thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 1 to 5 (e.g., from 2 to 5);
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
Ring C is

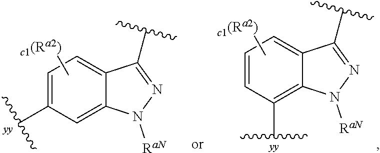

wherein: c1 is 0 or 1; R^{a2} is selected from the group consisting of: halo (e.g., —F) and C₁₋₃ alkyl optionally substituted with 1-3 F; and R^{aN} is C₁₋₃ alkyl.

In some embodiments, the compounds of Formula (I-A) are compounds of Formula (I-A-3):

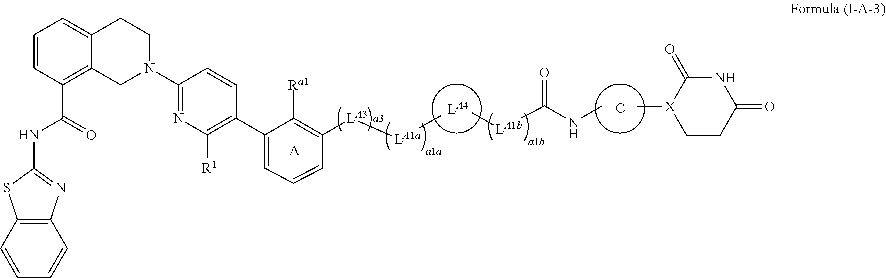

or pharmaceutically acceptable salts thereof, wherein:
R¹ is C(O)OH or C(O)OC₁₋₆ alkyl;
R^{a1} is C₁₋₃ alkyl optionally substituted with 1-3 F;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 1 to 5 (e.g., from 2 to 5);
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
Ring C is

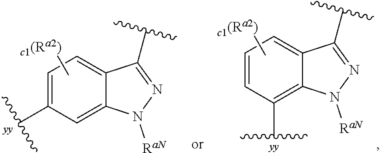

wherein: c1 is 0 or 1; R^{a2} is selected from the group consisting of: halo (e.g., —F) and C₁₋₃ alkyl optionally substituted with 1-3 F; and R^{aN} is C₁₋₃ alkyl.

In some embodiments of Formula (I-A-1), (I-A-2), or (I-A-3):
a3 is 1;
L^{A3} is —O—;
L^{A1a} and L^{A1b} are independently selected from the group consisting of: —CH₂—, —CH(Me)-, and —CF₂—; and
L^{A4} is 4-10 membered (e.g., nitrogen containing) heterocyclylene optionally substituted with 1-3 R^{a}.

In some embodiments of Formula (I-A-1), (I-A-2), or (I-A-3):
a3 is 1;
L^{A3} is —O—;
a1b is 1;
L^{A1b} is —CH₂—;
each L^{A1a} is independently selected from the group consisting of: —CH₂— and —CH(Me)-, provided that 0-1 L^{A1a} is —CH(Me)-; and
L^{A4} is 4-10 membered nitrogen-containing heterocyclylene optionally substituted with 1-2 substituents independently selected from the group consisting of: —F and C₁₋₃ alkyl optionally substituted with 1-3-F.

In some embodiments of Formula (I-A-1), (I-A-2), or (I-A-3):
a3 is 1;
L^{A3} is —O—;
a1b is 1;
L^{A1b} is —CH₂—;
a1a is 1, 2, or 3;
each L^{A1a} is independently selected from the group consisting of: —CH₂— and —CH(Me)-, provided that 0-1 L^{A1a} is —CH(Me)-; and
L^{A4} is selected from the group consisting of:

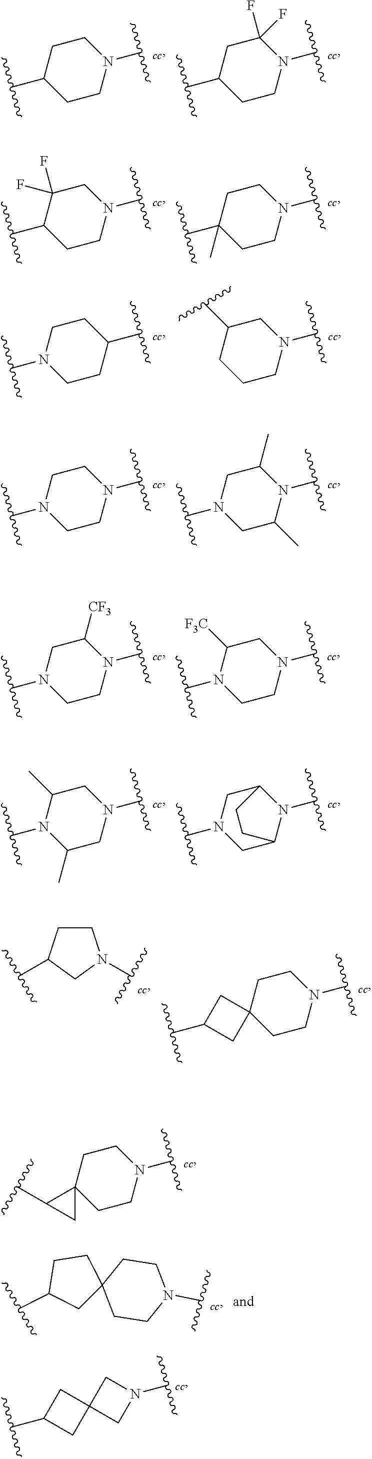

wherein cc represents the point of attachment to L^{A1b}.

In some embodiments of Formula (I-A), (I-A-1), (I-A-2), or (I-A-3), the

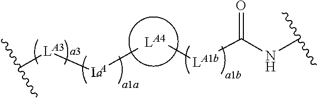

moiety is selected from the groups depicted in Table (L-I-A):

TABLE (L-I-A)

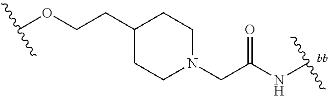

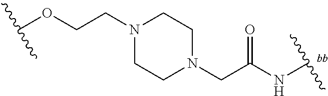

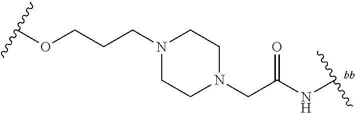

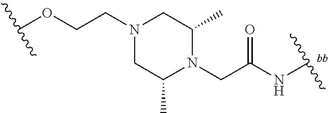

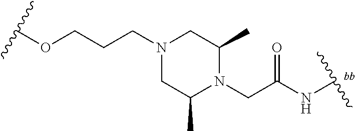

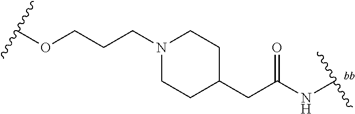

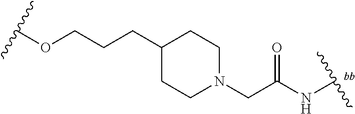

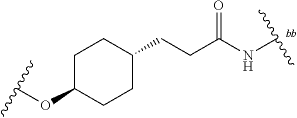

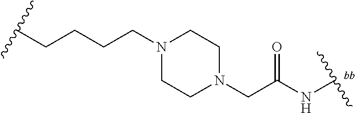

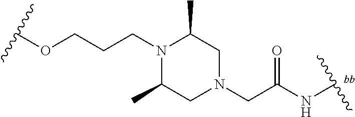

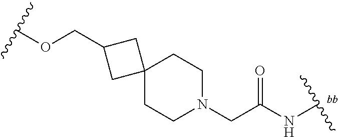

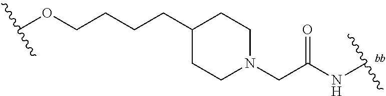

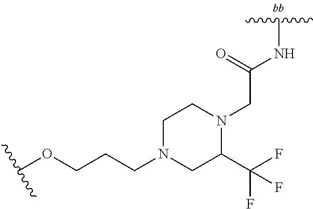

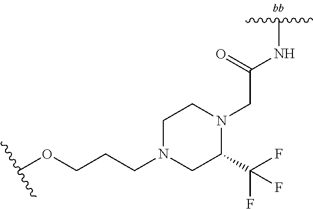

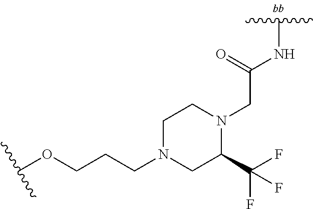

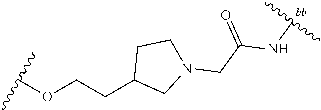

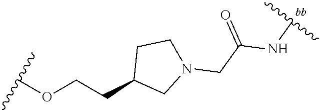

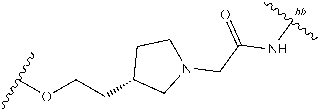

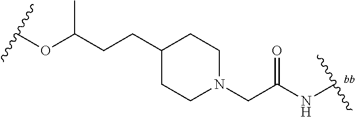

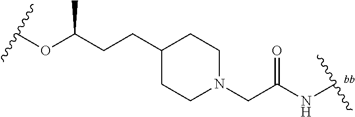

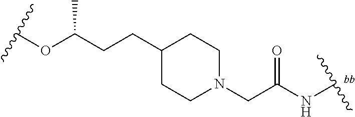

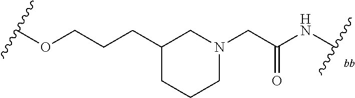

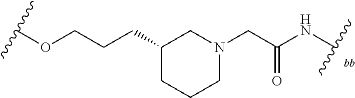

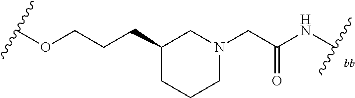

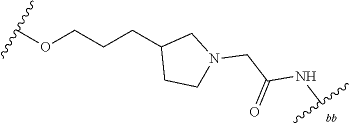

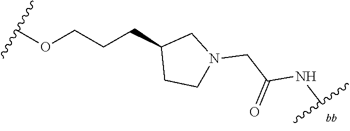

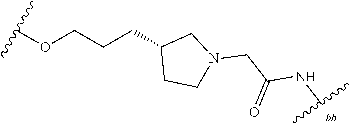

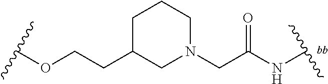

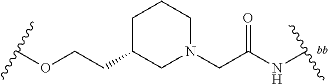

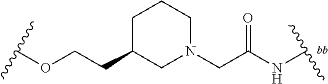

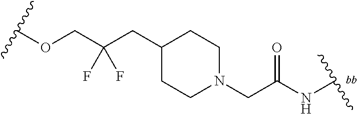

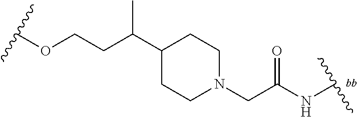

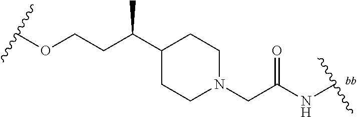

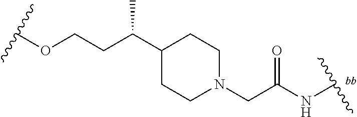

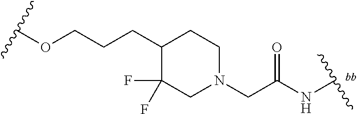

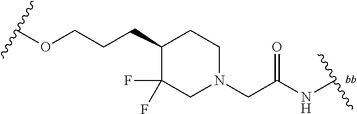

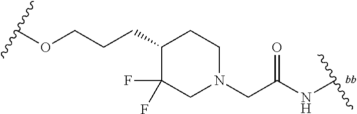

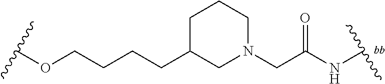

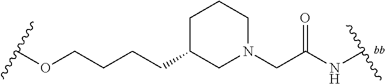

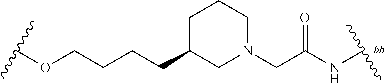

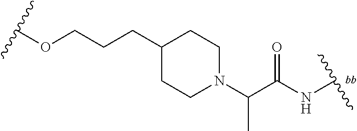

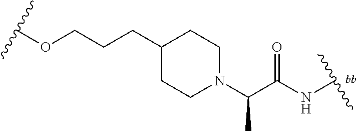

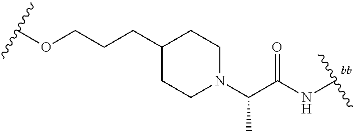

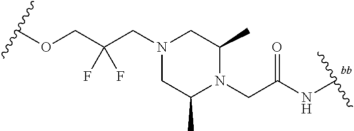

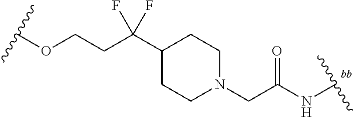

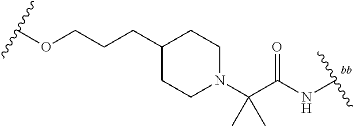

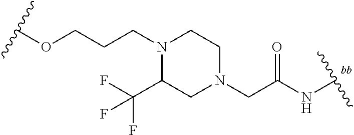

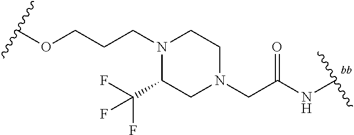

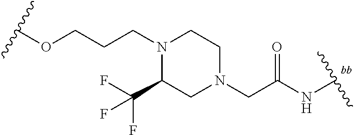

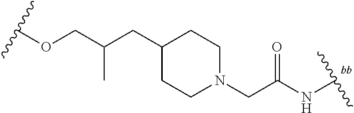

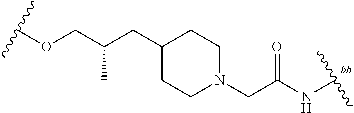

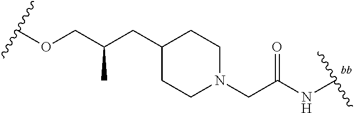

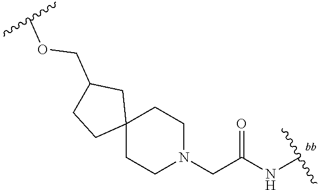

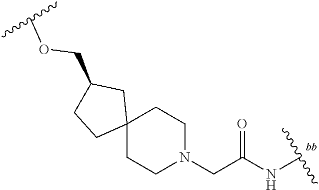

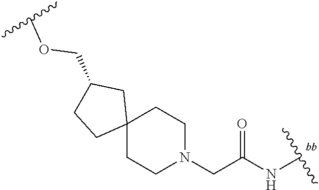

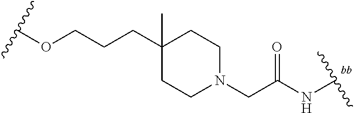

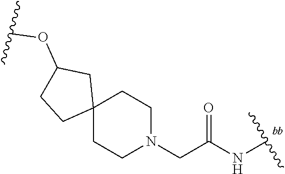

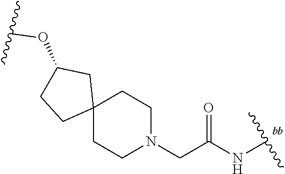

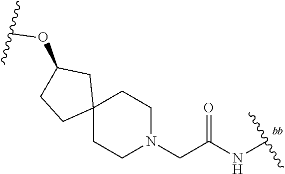

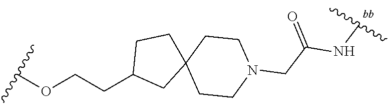

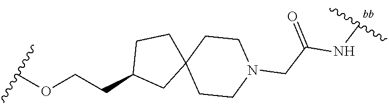

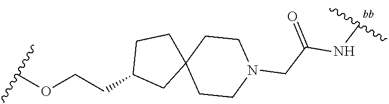

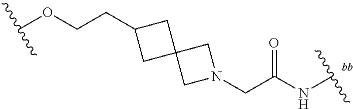

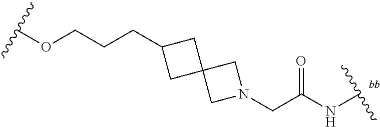

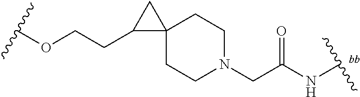

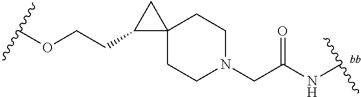

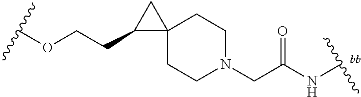

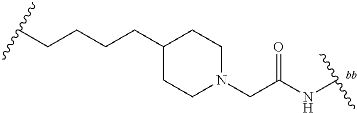

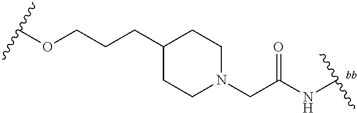

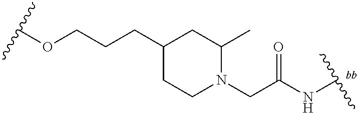

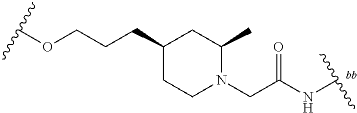

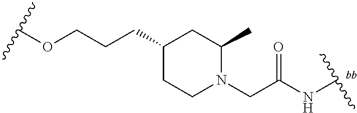

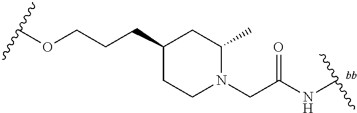

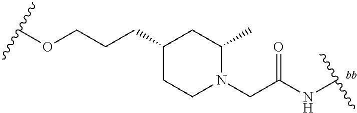

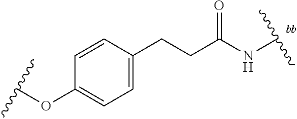

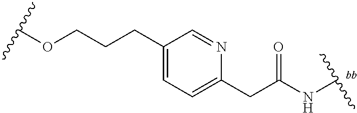

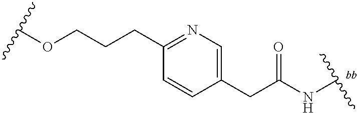

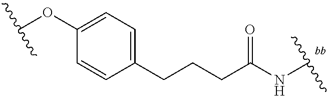

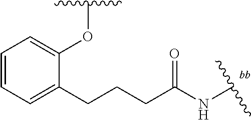

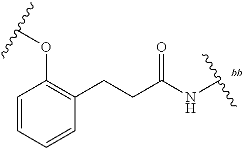

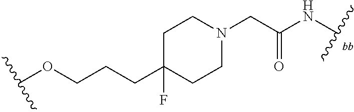

wherein bb represent the point of attachment to Ring C.

In some embodiments, the compounds of Formula (I-B) are compounds of Formula (I-B-1):

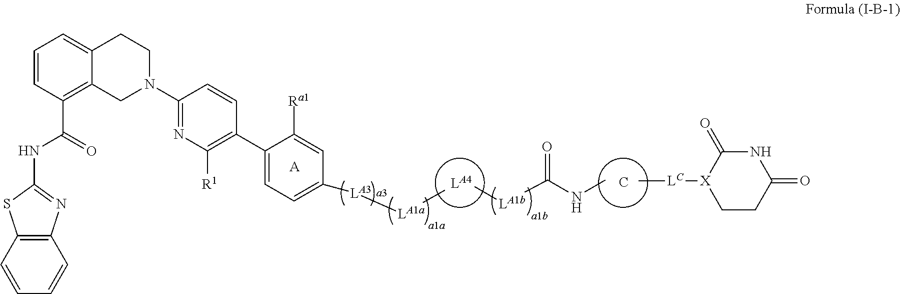

or pharmaceutically acceptable salts thereof, wherein:
R¹ is C(O)OH or C(O)OC₁₋₆ alkyl;
R^{a1} is C₁₋₃ alkyl optionally substituted with 1-3 F;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 1 to 5 (e.g., from 2 to 5);
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

In some embodiments, the compounds of Formula (I-B) are compounds of Formula (I-B-2):

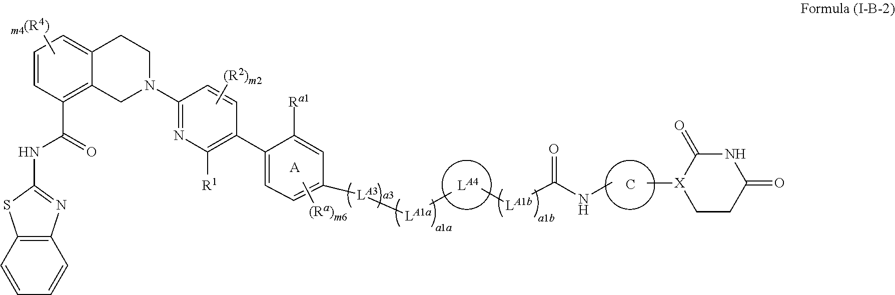

or pharmaceutically acceptable salts thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 1 to 5 (e.g., from 2 to 5);
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
Ring C is

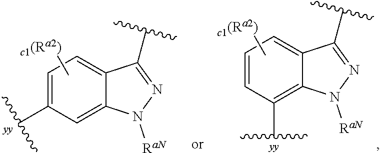

wherein: c1 is 0 or 1; R^{a2} is selected from the group consisting of: halo (e.g., —F) and C₁₋₃ alkyl optionally substituted with 1-3 F; and R^{aN} is C₁₋₃ alkyl.

In some embodiments, the compounds of Formula (I-B) are compounds of Formula (I-B-3):

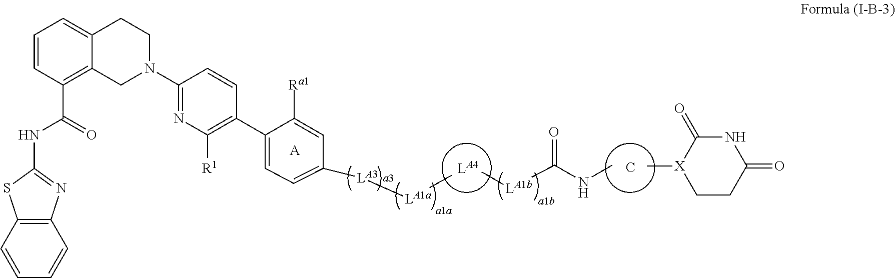

or pharmaceutically acceptable salts thereof, wherein:
R¹ is C(O)OH or C(O)OC₁₋₆ alkyl;
R^{a1} is C₁₋₃ alkyl optionally substituted with 1-3 F;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 1 to 5 (e.g., from 2 to 5);
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
Ring C is

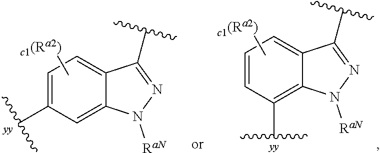

wherein: c1 is 0 or 1; R^{a2} is selected from the group consisting of: halo (e.g., —F) and C₁₋₃ alkyl optionally substituted with 1-3 F; and R^{aN} is C₁₋₃ alkyl.

In some embodiments of Formula (I-B), (I-B-1), (I-B-2), or (I-B-3):
a3 is 1;
L^{A3} is —O—;
L^{A1a} and L^{A1b} are independently selected from the group consisting of: —CH₂—, —CH(Me)-, and —CF₂—; and
L^{A4} is 4-10 membered (e.g., nitrogen containing) heterocyclylene optionally substituted with 1-3 R^{a}.

In some embodiments of Formula (I-B-1), (I-B-2), or (I-B-3):
a3 is 1;
L^{A3} is —O—;
a1b is 1;
L^{A1b} is —CH₂—;
each L^{A1a} is independently selected from the group consisting of: —CH₂— and —CH(Me)-, provided that 0-1 L^{A1a} is —CH(Me)-; and
L^{A4} is 4-10 membered nitrogen-containing heterocyclylene optionally substituted with 1-2 substituents independently selected from the group consisting of: —F and C₁₋₃ alkyl optionally substituted with 1-3-F.

In some embodiments of Formula (I-B-1), (I-B-2), or (I-B-3):
a3 is 1;
L^{A3} is —O—;
a1b is 1;
L^{A1b} is —CH₂—;
a1a is 1, 2, or 3;
each L^{A1a} is independently selected from the group consisting of: —CH₂— and —CH(Me)-, provided that 0-1 L^{A1a} is —CH(Me)-; and
L^{A4} is selected from the group consisting of:

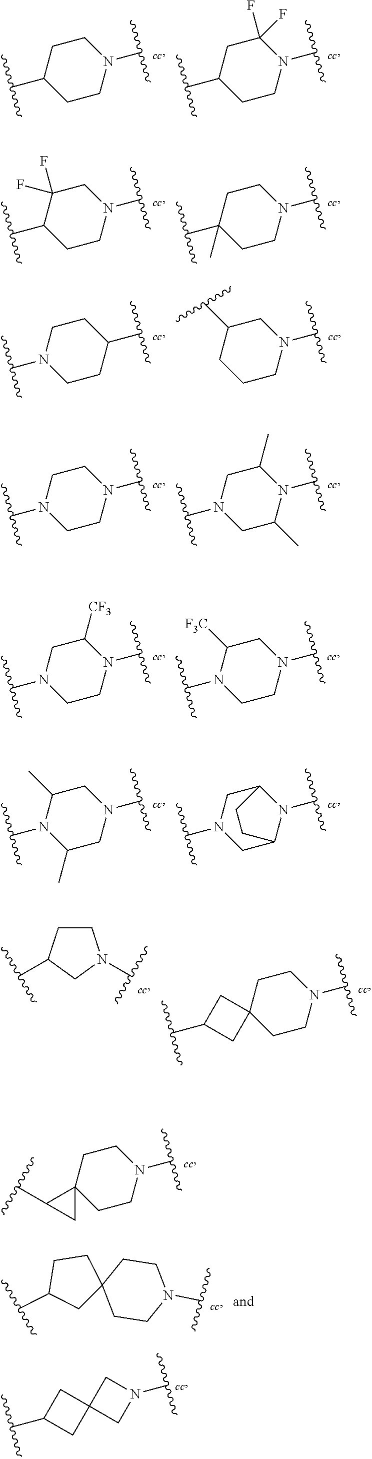

wherein cc represents the point of attachment to L^{A1b}.

In some embodiments of Formula (I-B-1), (I-B-2), or (I-B-3), the

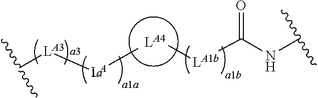

moiety is selected from the groups depicted in Table (L-I-B):

TABLE (L-I-B)

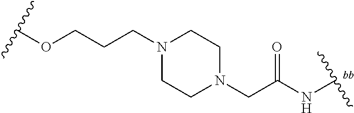

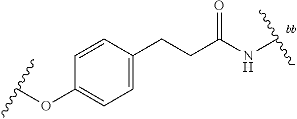

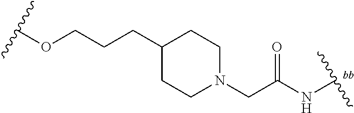

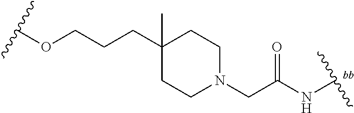

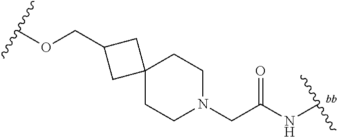

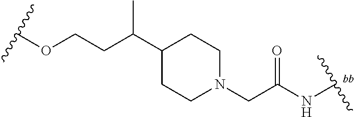

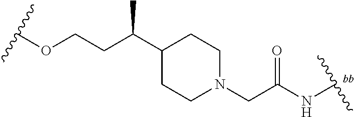

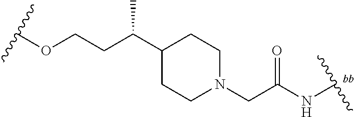

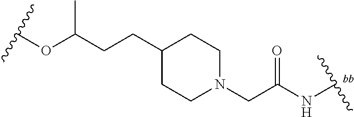

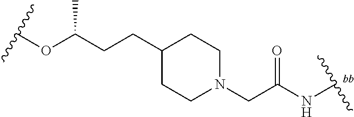

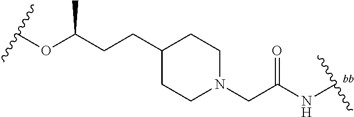

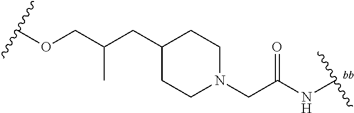

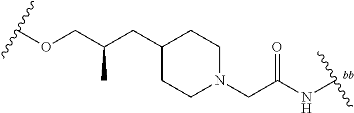

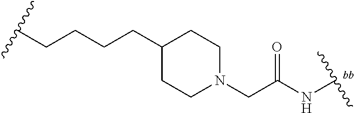

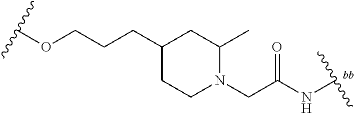

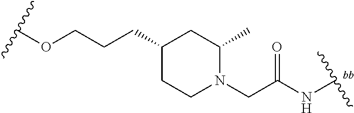

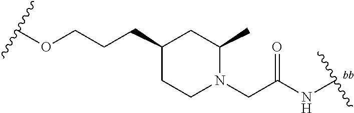

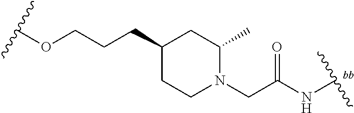

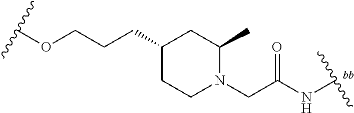

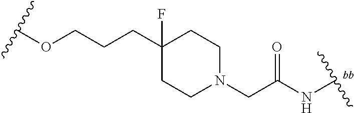

wherein bb represents the point of attachment to Ring C.

In some embodiments, the compounds of Formula (I-E) are compounds of Formula (I-E-1):

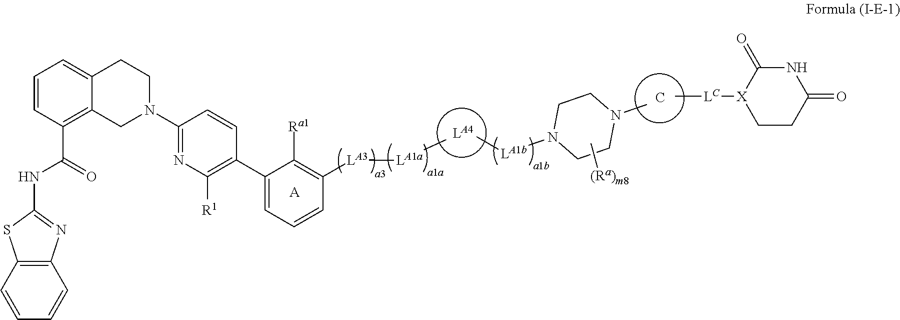

or pharmaceutically acceptable salts thereof, wherein:
R¹ is C(O)OH or C(O)OC₁₋₆ alkyl;
R^{a1} is C₁₋₃ alkyl optionally substituted with 1-3 F;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
m8 is 0, 1, or 2.

In some embodiments, the compounds of Formula (I-E) are compounds of Formula (I-E-2):

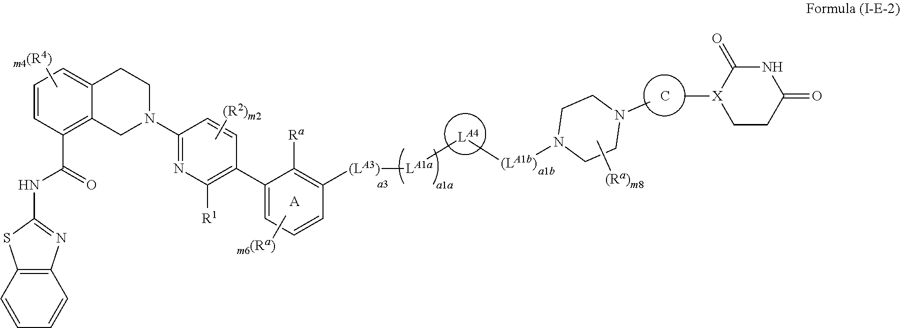

or pharmaceutically acceptable salts thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
m8 is 0, 1, or 2; and
Ring C is

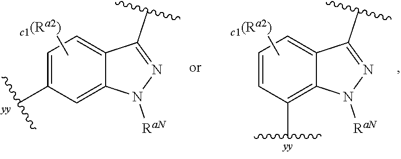

wherein: c1 is 0 or 1; R^{a2} is selected from the group consisting of: halo (e.g., —F) and C₁₋₃ alkyl optionally substituted with 1-3 F; and R^{aN} is C₁₋₃ alkyl.

In some embodiments, the compounds of Formula (I-E) are compounds of Formula (I-E-3):

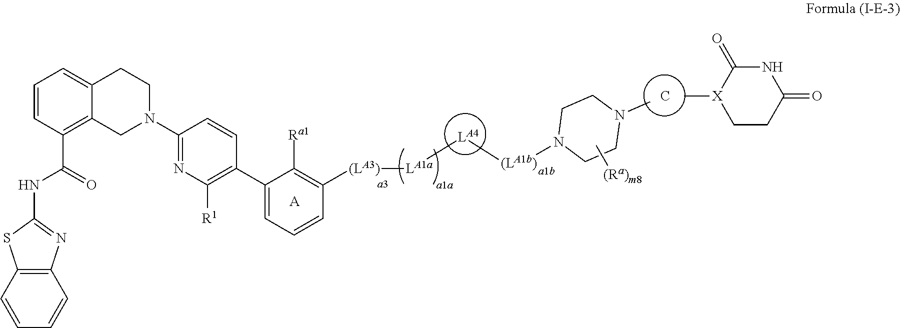

or pharmaceutically acceptable salts thereof, wherein:
R¹ is C(O)OH or C(O)OC₁₋₆ alkyl;
R^{a1} is C₁₋₃ alkyl optionally substituted with 1-3 F;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
m8 is 0, 1, or 2; and
Ring C is

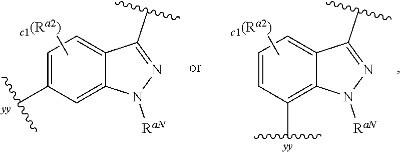

wherein: c1 is 0 or 1; R^{a2} is selected from the group consisting of: halo (e.g., —F) and C₁₋₃ alkyl optionally substituted with 1-3 F; and R^{aN} is C₁₋₃ alkyl.

In some embodiments of Formula (I-E-1), (I-E-2), or (I-E-3):
a3 is 1;
L^{A3} is —O—;
a1a is 0; and
L^{A4} is selected from the group consisting of:
C₃₋₁₀ cycloalkylene optionally substituted with 1-3 R^{a}; and
phenylene optionally substituted with 1-3 R^{a}.

In some embodiments of Formula (I-E-1), (I-E-2), or (I-E-3):
a3 is 1;
L^{A3} is —O—;
a1a is 0;
a1b is 3 or 4;
each L^{A1b} is independently selected from the group consisting of: —CH₂— and —CH(Me)-, provided that 0-1 of L^{A1b} is —CH(Me)-; and
L^{A4} is selected from the group consisting of:

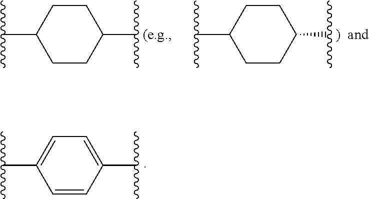

In some embodiments of Formula (I-E-1), (I-E-2), or (I-E-3):
a3 is 1;
L^{A3} is —O—;
a1a is 2, 3, or 4; and
L^{A4} is 4-10 membered heterocyclylene optionally substituted with 1-3 R^{a}.

In some embodiments of Formula (I-E-1), (I-E-2), or (I-E-3):
a3 is 1;
L^{A3} is —O—;
a1a is 2, 3, or 4;
each L^{A1a} is CH₂;
a1b is 0, 1, or 2;
each L^{A1b} is CH₂; and
L^{A4} is 4-8 membered nitrogen-containing heterocyclylene optionally substituted with 1-3 R^{a}.

In some embodiments of Formula (I-E-1), (I-E-2), or (I-E-3):
a3 is 1;
L^{A3} is —O—;
a1a is 2, 3, or 4;
each L^{A1a} is CH₂;
a1b is 0, 1, or 2;
each L^{A1b} is CH₂; and
L^{A4} is selected from the group consisting of:

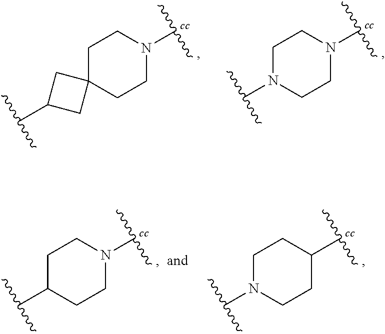

wherein cc represents the point of attachment to L^{A1b}.

In some embodiments of Formula (I-E), (I-E-1), (I-E-2), or (I-E-3), the

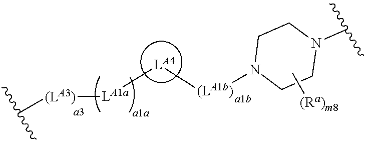

moiety is selected from the groups depicted in Table (L-I-E):

TABLE (L-I-E)

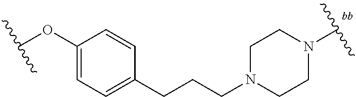

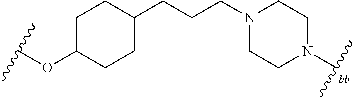

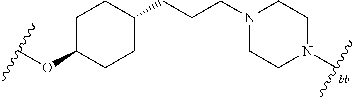

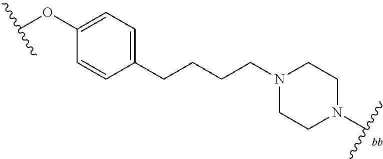

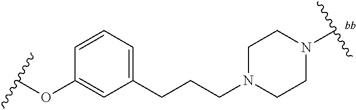

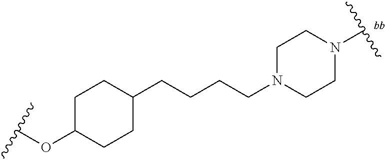

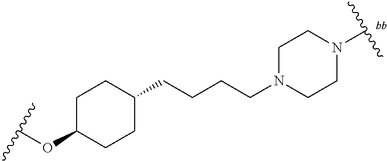

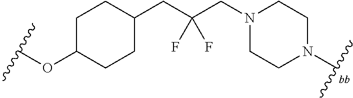

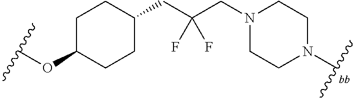

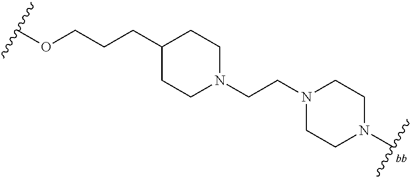

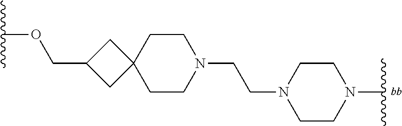

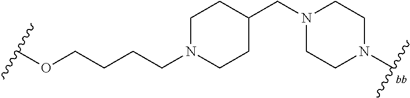

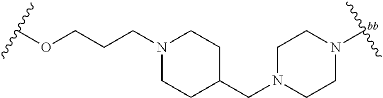

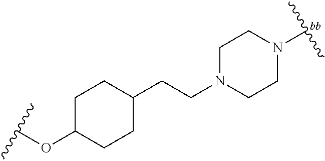

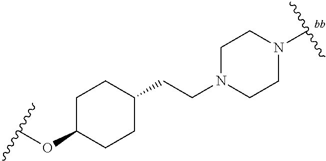

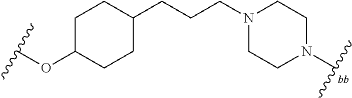

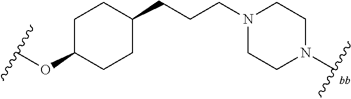

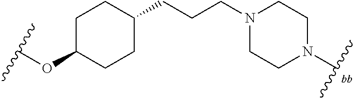

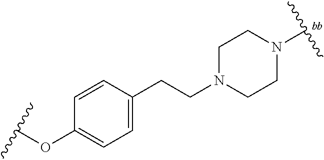

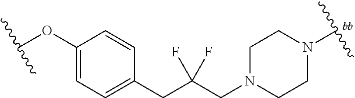

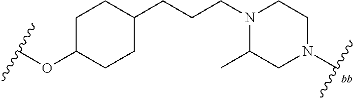

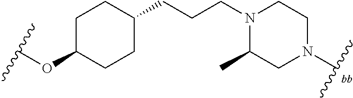

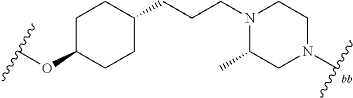

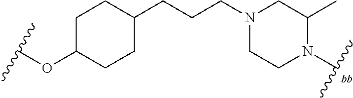

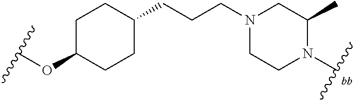

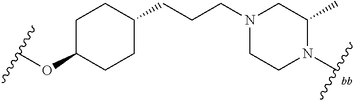

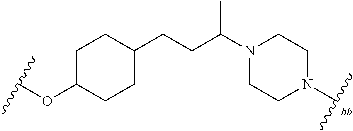

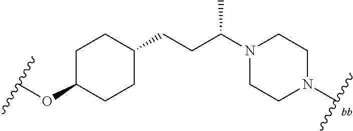

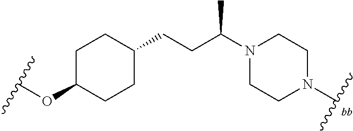

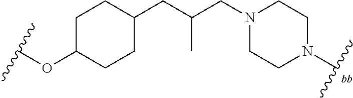

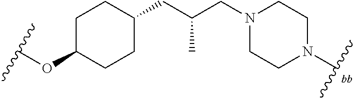

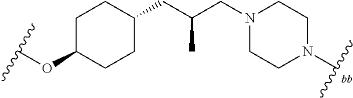

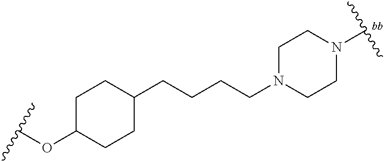

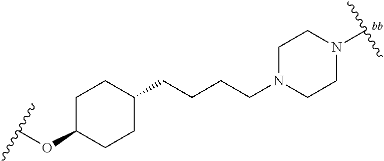

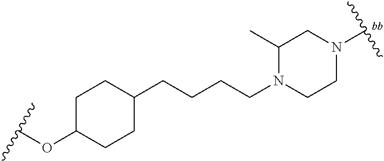

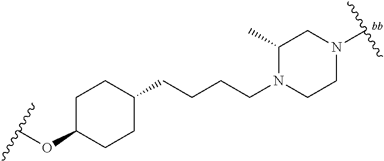

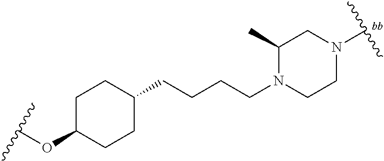

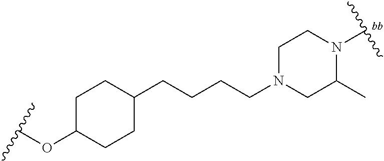

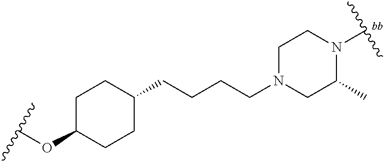

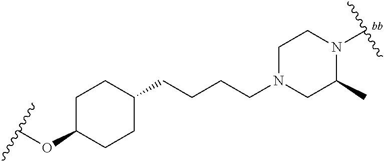

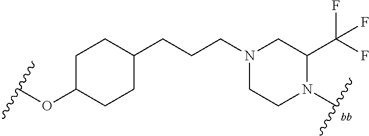

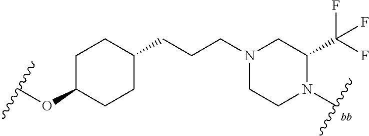

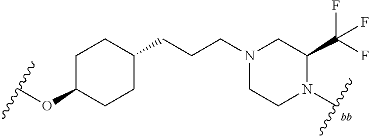

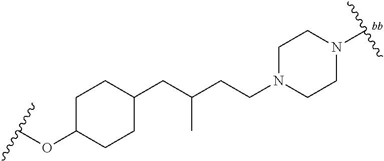

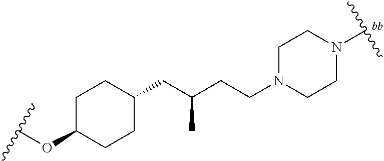

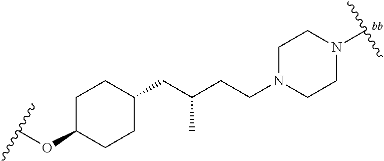

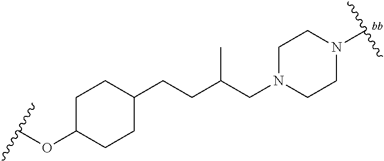

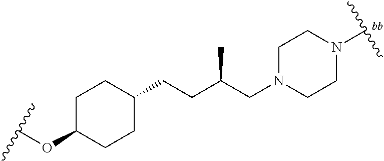

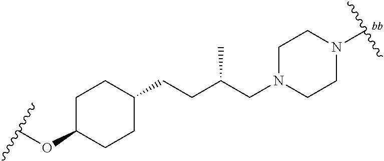

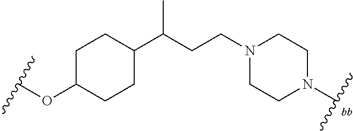

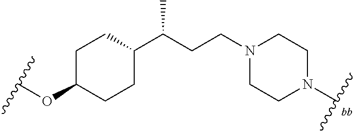

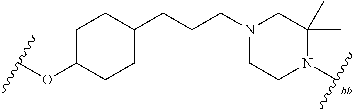

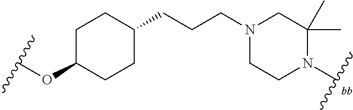

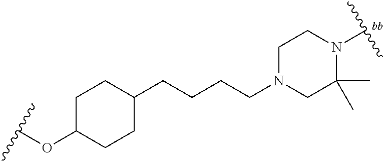

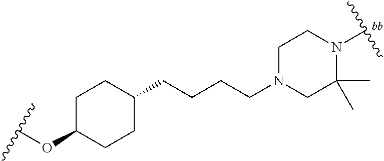

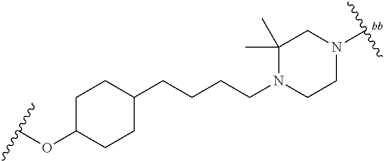

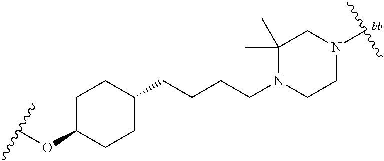

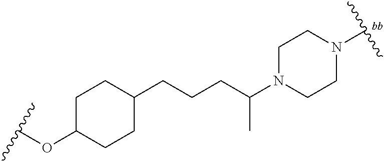

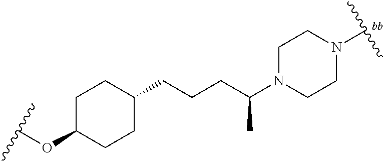

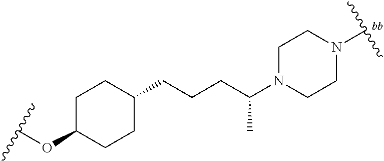

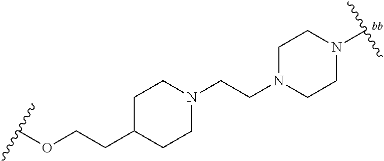

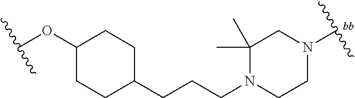

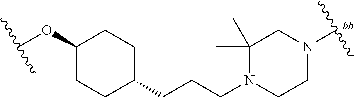

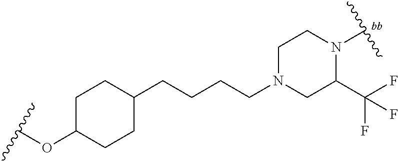

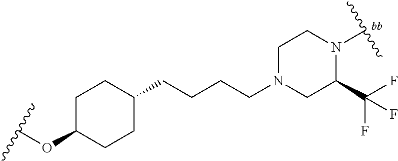

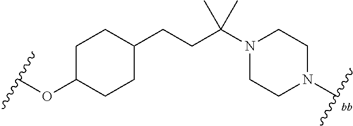

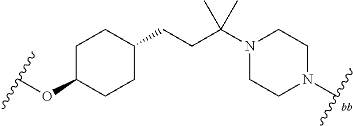

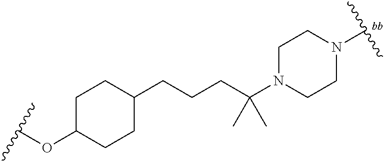

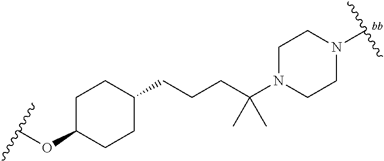

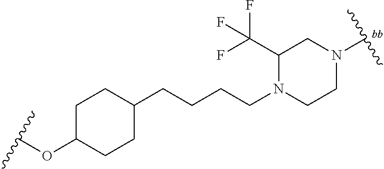

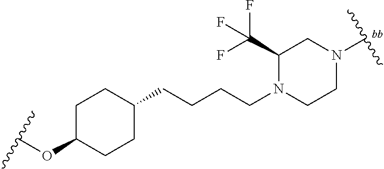

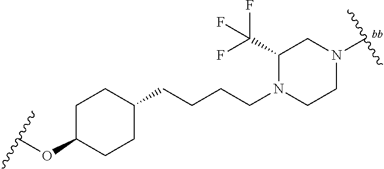

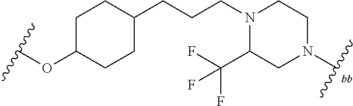

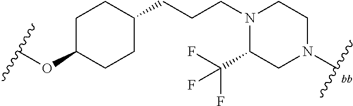

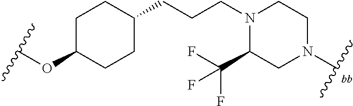

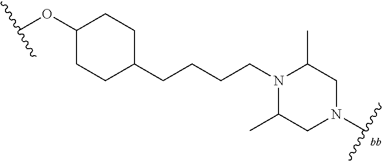

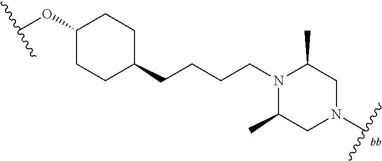

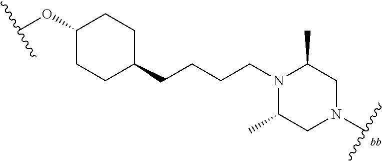

wherein bb represents the point of attachment to Ring C.

In some embodiments, the compounds of Formula (I-Ea) are compounds of Formula (I-Ea-1):

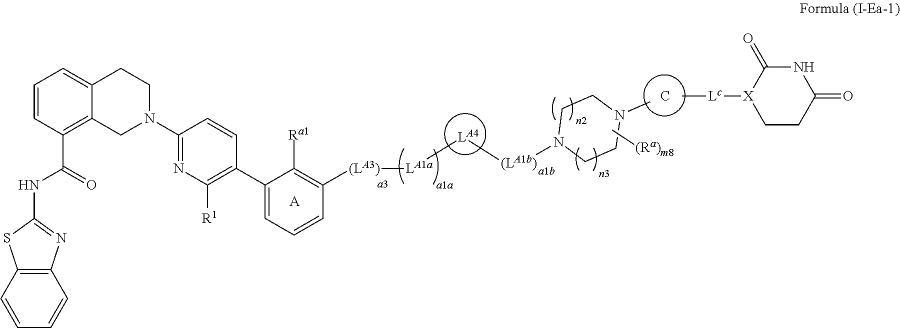

or pharmaceutically acceptable salts thereof, wherein:
R¹ is C(O)OH or C(O)OC₁₋₆ alkyl;
R^{a1} is C₁₋₃ alkyl optionally substituted with 1-3 F;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
n2 and n3 are independently 0, 1, or 2; and
m8 is 0, 1, or 2.

In some embodiments, the compounds of Formula (I-Ea) are compounds of Formula (I-Ea-2):

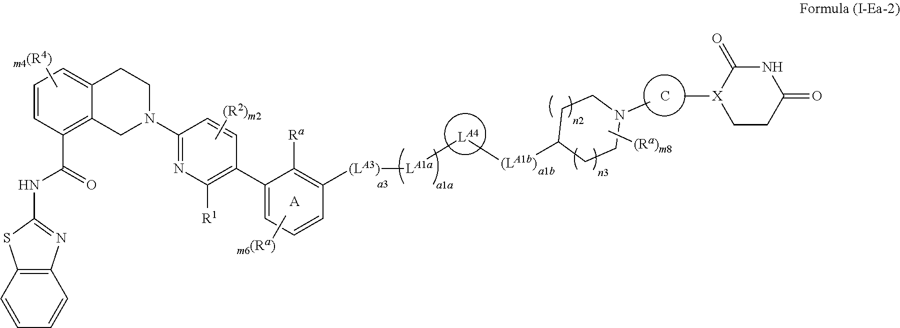

or pharmaceutically acceptable salts thereof, wherein: PGP
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
n2 and n3 are independently 0, 1, or 2;
m8 is 0, 1, or 2; and
Ring C is

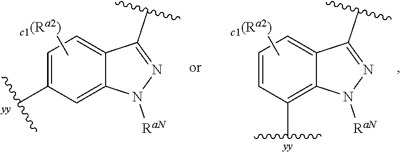

wherein: c1 is 0 or 1; R^{a2} is selected from the group consisting of: halo (e.g., —F) and C₁₋₃ alkyl optionally substituted with 1-3 F; and R^{aN} is C₁₋₃ alkyl.

In some embodiments, the compounds of Formula (I-Ea) are compounds of Formula (I-Ea-3):

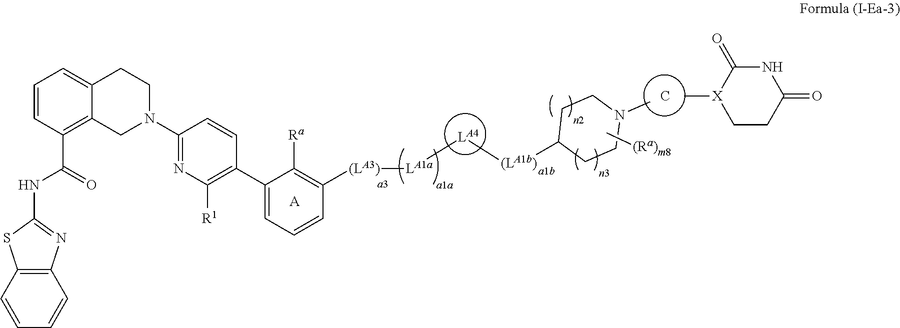

or pharmaceutically acceptable salts thereof, wherein:
R¹ is C(O)OH or C(O)OC₁₋₆ alkyl;
R^{a1} is C₁₋₃ alkyl optionally substituted with 1-3 F;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
m8 is 0, 1, or 2;
n2 and n3 are independently 0, 1, or 2; and
Ring C is

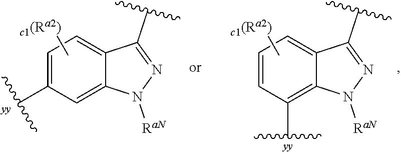

wherein: c1 is 0 or 1; R^{a2} is selected from the group consisting of: halo (e.g., —F) an C₁₋₃ alkyl optionally substituted with 1-3 F; and R^{aN} is C₁₋₃ alkyl.

In some embodiments of Formula (I-Ea-1), (I-Ea-2), or (I-Ea-3):
a3 is 1;
L^{A3} is —O—;
a1a is 0; and
L^{A4} is selected from the group consisting of:
C₃₋₁₀ cycloalkylene optionally substituted with 1-3 R^{a}; and
phenylene optionally substituted with 1-3 R^{a}.

In some embodiments of Formula (I-Ea-1), (I-Ea-2), or (I-Ea-3):
a3 is 1;
L^{A3} is —O—;
a1a is 0;
a1b is 3 or 4;
each L^{A1b} is independently selected from the group consisting of: —CH₂— and —CH(Me)-, provided that 0-1 of L^{A1b} is —CH(Me)-; and
L^{A4} is selected from the group consisting of:

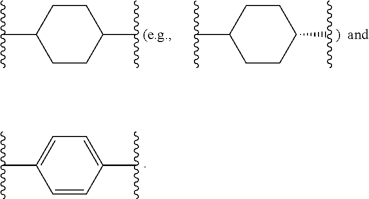

In some embodiments of Formula (I-Ea-1), (I-Ea-2), or (I-Ea-3):
a3 is 1;
L^{A3} is —O—;
a1a is 2, 3, or 4; and
L^{A4} is 4-10 membered heterocyclylene optionally substituted with 1-3 R^{a}.

In some embodiments of Formula (I-Ea-1), (I-Ea-2), or (I-Ea-3):
a3 is 1;
L^{A3} is —O—;
a1a is 2, 3, or 4;
each L^{A1a} is CH₂;
a1b is 0, 1, or 2;
each L^{A1b} is CH₂; and
L^{A4} is 4-8 membered nitrogen-containing heterocyclylene optionally substituted with 1-3 R^{a}.

In some embodiments of Formula (I-Ea-1), (I-Ea-2), or (I-Ea-3):
a3 is 1;
L^{A3} is —O—;
a1a is 2, 3, or 4;
each L^{A1a} is CH₂;
a1b is 0, 1, or 2;
each L^{A1b} is CH₂; and
L^{A4} is selected from the group consisting of:

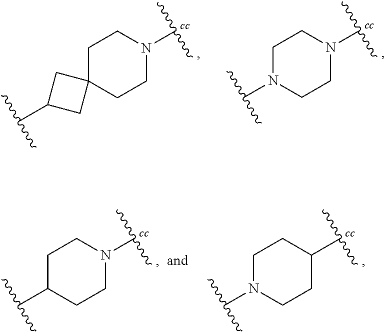

wherein cc represents the point of attachment to L^{A1b}.

In some embodiments of Formula (I-Ea-1), (I-Ea-2), or (I-Ea-3), n2 is 1; and n3 is 1.

In some embodiments of Formula (I-Ea-1), (I-Ea-2), or (I-Ea-3), the

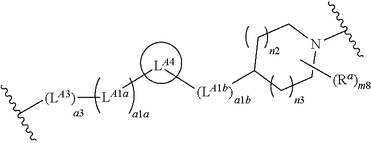

moiety is selected from the groups depicted in Table (L-I-Ea):

TABLE (L-I-Ea)

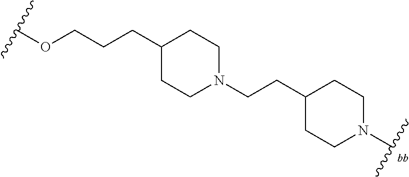

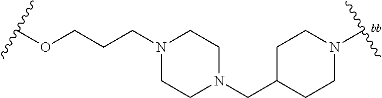

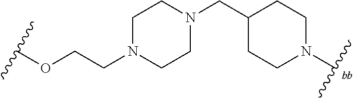

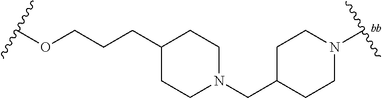

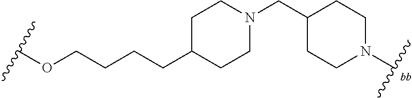

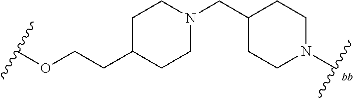

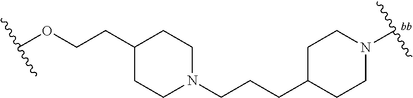

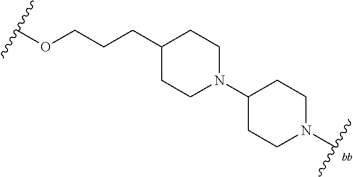

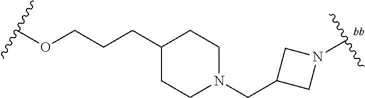

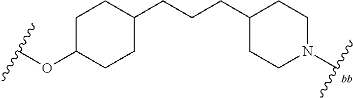

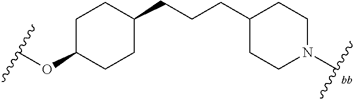

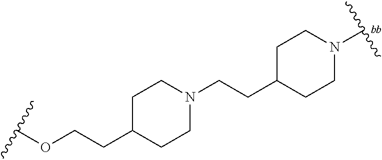

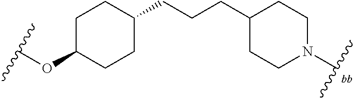

wherein bb represents the point of attachment to Ring C.

In some embodiments, the compounds of Formula (I-Eb) are compounds of Formula (I-Eb-1):

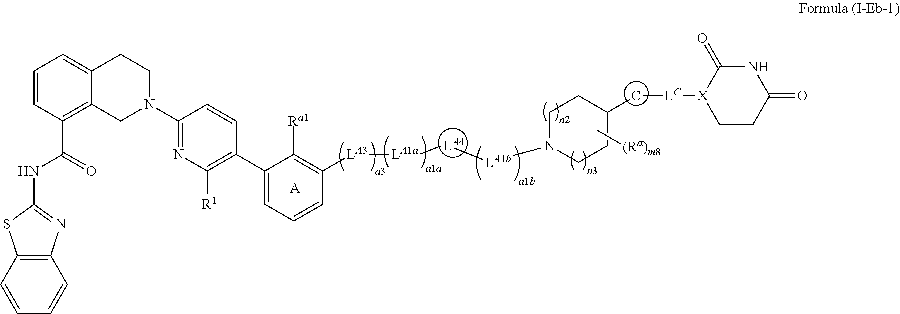

or pharmaceutically acceptable salts thereof, wherein:
R¹ is C(O)OH or C(O)OC₁₋₆ alkyl;
R^{a1} is C₁₋₃ alkyl optionally substituted with 1-3 F;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
n2 and n3 are independently 0, 1, or 2; and
m8 is 0, 1, or 2.

In some embodiments, the compounds of Formula (I-Eb) are compounds of Formula (I-Eb-2):

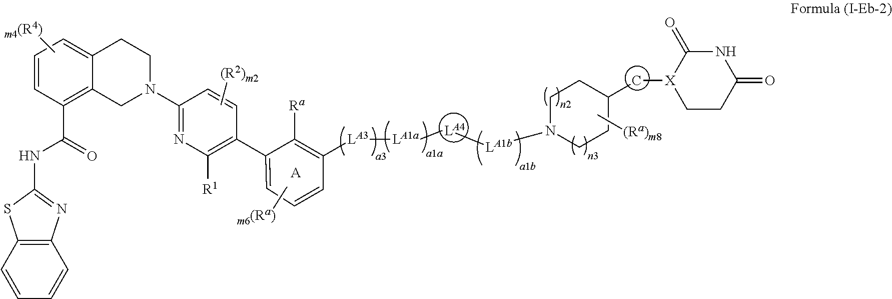

or pharmaceutically acceptable salts thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
m8 is 0, 1, or 2;
n2 and n3 are independently 0, 1, or 2; and
Ring C is

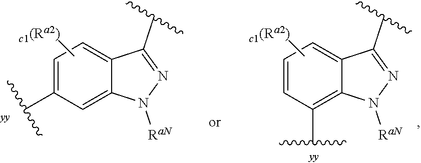

wherein: c1 is 0 or 1; R^{a2} is selected from the group consisting of: halo (e.g., —F) and C₁₋₃ alkyl optionally substituted with 1-3 F; and R^{aN} is C₁₋₃ alkyl.

In some embodiments, the compounds of Formula (I-Eb) are compounds of Formula (I-Eb-3):

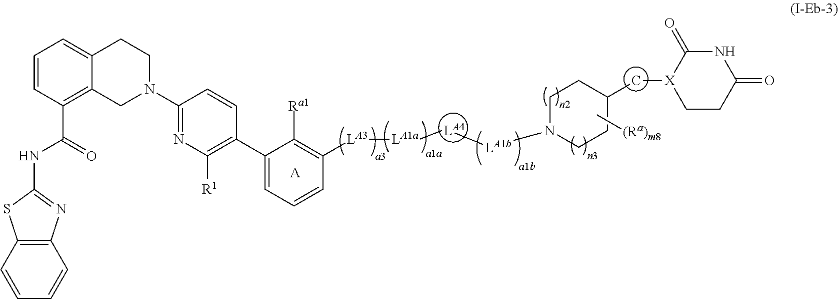

or pharmaceutically acceptable salts thereof, wherein:
R¹ is C(O)OH or C(O)OC₁₋₆ alkyl;
R^{a1} is C₁₋₃ alkyl optionally substituted with 1-3 F;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
m8 is 0, 1, or 2;
n2 and n3 are independently 0, 1, or 2; and
Ring C is

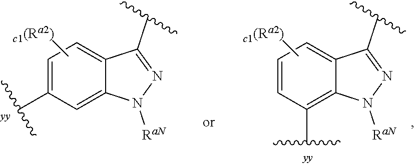

wherein: c1 is 0 or 1; R^{a2} is selected from the group consisting of: halo (e.g., —F) and C₁₋₃ alkyl optionally substituted with 1-3 F; and R^{aN} is C₁₋₃ alkyl.

In some embodiments of Formula (I-Eb-1), (I-Eb-2), or (I-Eb-3):
a3 is 1;
L^{A3} is —O—;
a1a is 0; and
L^{A4} is selected from the group consisting of:
C₃₋₁₀ cycloalkylene optionally substituted with 1-3 R^{a}; and
phenylene optionally substituted with 1-3 R^{a}.

In some embodiments of Formula (I-Eb-1), (I-Eb-2), or (I-Eb-3):
a3 is 1;
L^{A3} is —O—;
a1a is 0;
a1b is 3 or 4;
each L^{A1b} is independently selected from the group consisting of: —CH₂— and —CH(Me)-, provided that 0-1 of L^{A1b} is —CH(Me)-; and
L^{A4} is selected from the group consisting of:

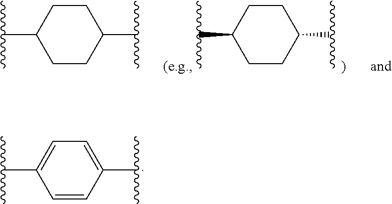 [SMILES: *C1CCC(*)CC1 ; *[C@H]1CC[C@H](*)CC1 ; *c1ccc(*)cc1]

In some embodiments of Formula (I-Eb-1), (I-Eb-2), or (I-Eb-3):
a3 is 1;
L^{A3} is —O—;
a1a is 2, 3, or 4; and
L^{A4} is 4-10 membered heterocyclylene optionally substituted with 1-3 R^{a}.

In some embodiments of Formula (I-Eb-1), (I-Eb-2), or (I-Eb-3):
a3 is 1;
L^{A3} is —O—;
a1a is 2, 3, or 4;
each L^{A1a} is CH₂;
a1b is 0, 1, or 2;
each L^{A1b} is CH₂; and
L^{A4} is 4-8 membered nitrogen-containing heterocyclylene optionally substituted with 1-3 R^{a}.

In some embodiments of Formula (I-Eb-1), (I-Eb-2), or (I-Eb-3):
a3 is 1;
L^{A3} is —O—;
a1a is 2, 3, or 4;
each L^{A1a} is CH₂;
a1b is 0, 1, or 2;
each L^{A1b} is CH₂; and
L^{A4} is selected from the group consisting of:

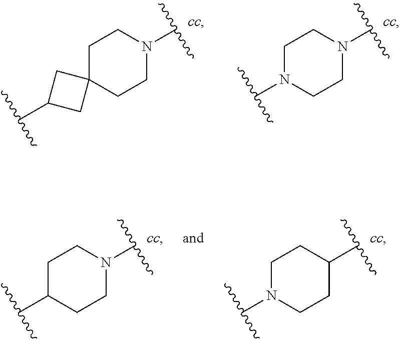

wherein cc represents the point of attachment to L^{A1b}.

In some embodiments of Formula (I-Eb-1), (I-Eb-2), or (I-Eb-3), n2 is 1; and n3 is 1.

In some embodiments of Formula (I-Eb-1), (I-Eb-2), or (I-Eb-3), the

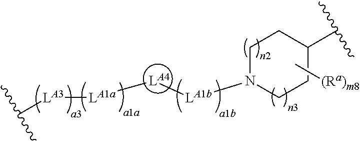

moiety is selected from the groups depicted in Table (L-I-Eb):

Table (L-I-Eb):

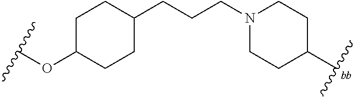

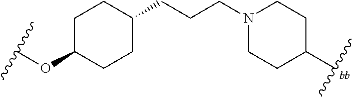

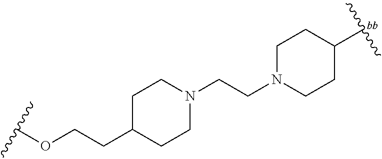

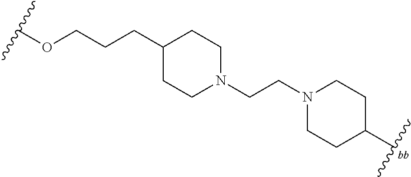

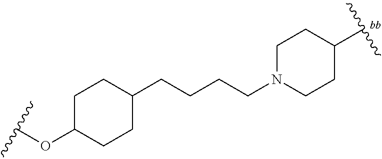

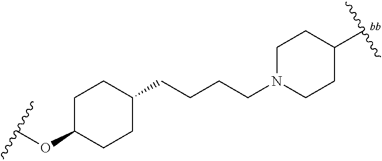

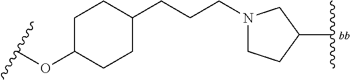

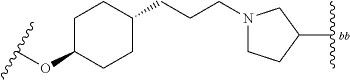

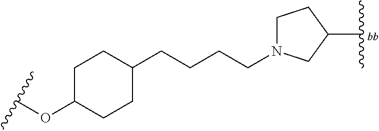

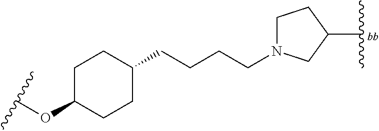

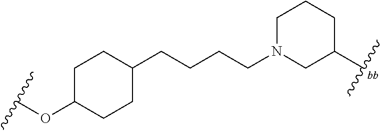

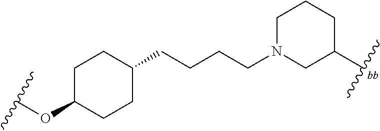

wherein bb represents the point of attachment to Ring C.

In some embodiments, the compounds of Formula (I-F) are compounds of Formula (I-F-1):

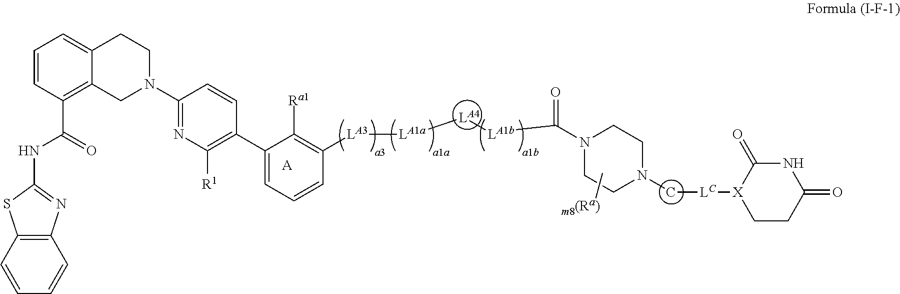

or pharmaceutically acceptable salts thereof, wherein:
R¹ is C(O)OH or C(O)OC₁₋₆ alkyl;
R^{a1} is C₁₋₃ alkyl optionally substituted with 1-3 F;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
m8 is 0, 1, or 2.

In some embodiments, the compounds of Formula (I-F) are compounds of Formula (I-F-2):

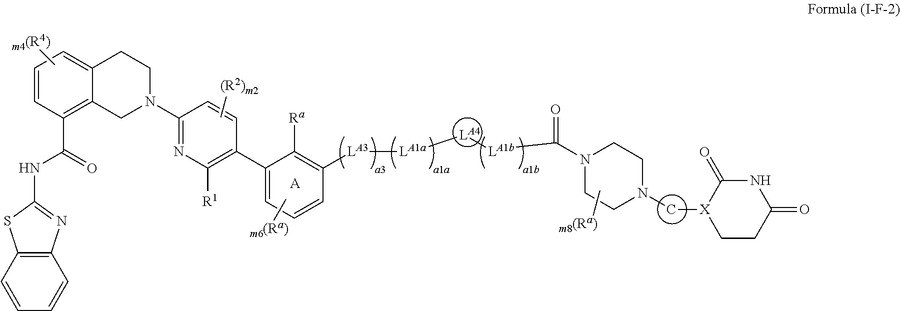

or pharmaceutically acceptable salts thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
m8 is 0, 1, or 2; and
Ring C is

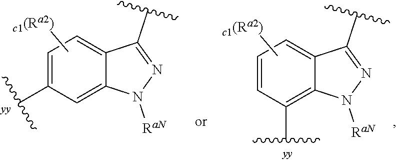

wherein: c1 is 0 or 1; R^{a2} is selected from the group consisting of: halo (e.g., —F) and C₁₋₃ alkyl optionally substituted with 1-3 F; and R^{aN} is C₁₋₃ alkyl.

In some embodiments, the compounds of Formula (I-F) are compounds of Formula (I-F-3):

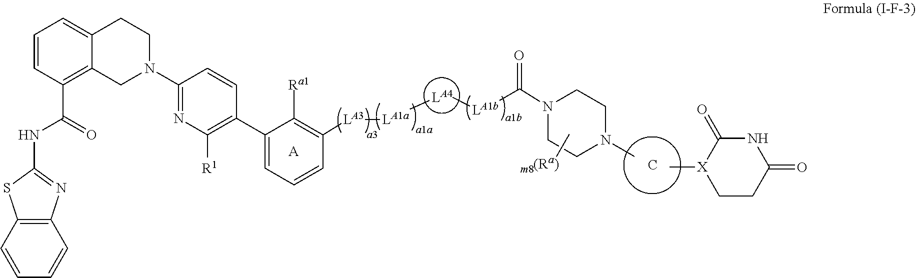

or pharmaceutically acceptable salts thereof, wherein:
R¹ is C(O)OH or C(O)OC₁₋₆ alkyl;
R^{a1} is C₁₋₃ alkyl optionally substituted with 1-3 F;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
m8 is 0, 1, or 2; and
Ring C is

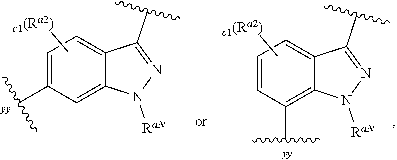

wherein: c1 is 0 or 1; R^{a2} is selected from the group consisting of: halo (e.g., —F) and C₁₋₃ alkyl optionally substituted with 1-3 F; and R^{aN} is C₁₋₃ alkyl.

In some embodiments of Formula (I-F-1), (I-F-2), or (I-F-3):
a3 is 1;
L^{A3} is —O—;
a1a is 0; and
L^{A4} is selected from the group consisting of:
C₃₋₁₀ cycloalkylene optionally substituted with 1-3 R^{a}; and
phenylene optionally substituted with 1-3 R^{a}.

In some embodiments of Formula (I-F-1), (I-F-2), or (I-F-3):
a3 is 1;
L^{A3} is —O—;
a1a is 0;
a1b is 3 or 4;
each L^{A1b} is independently selected from the group consisting of: —CH₂— and —CH(Me)-, provided that 0-1 of L^{A1b} is —CH(Me)-; and
L^{A4} is selected from the group consisting of:

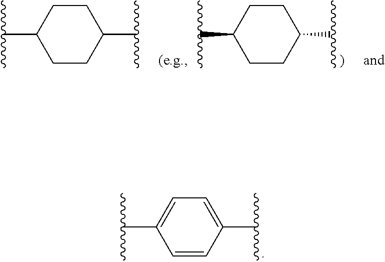 [SMILES: *C1CCC(*)CC1 ; *[C@H]1CC[C@H](*)CC1 ; *c1ccc(*)cc1]

In some embodiments of Formula (I-F-1), (I-F-2), or (I-F-3):
a3 is 1;
L^{A3} is —O—;
a1a is 2, 3, or 4; and
L^{A4} is 4-10 membered heterocyclylene optionally substituted with 1-3 R^{a}.

In some embodiments of Formula (I-F-1), (I-F-2), or (I-F-3):
a3 is 1;
L^{A3} is —O—;
a1a is 2, 3, or 4;
each L^{A1a} is CH₂;
a1b is 0, 1, or 2;
each L^{A1b} is CH₂; and
L^{A4} is 4-8 membered nitrogen-containing heterocyclylene optionally substituted with 1-3 R^{a}.

In some embodiments of Formula (I-F-1), (I-F-2), or (I-F-3), the

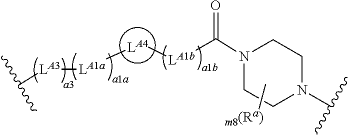

moiety is selected from the groups depicted in Table (L-I-F):

Table (L-I-F)

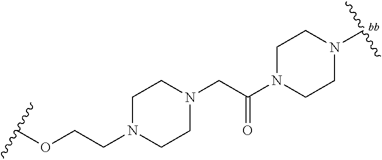

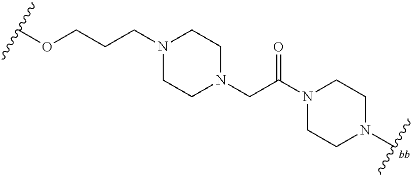

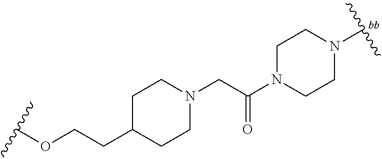

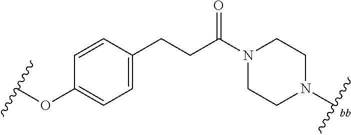

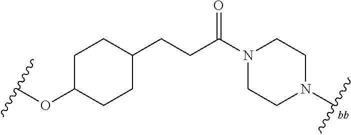

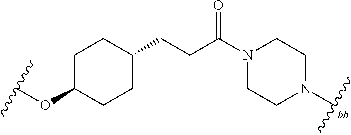

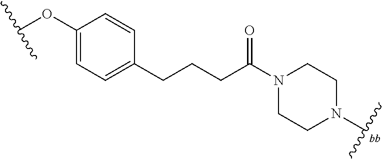

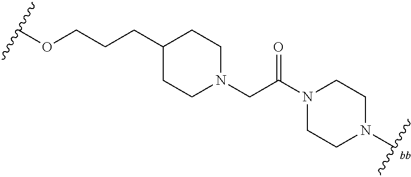

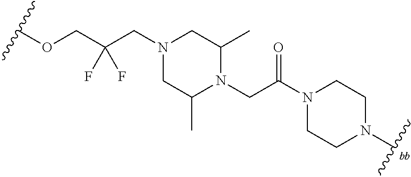

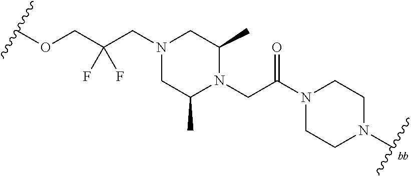

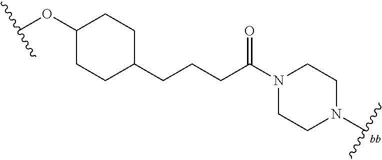

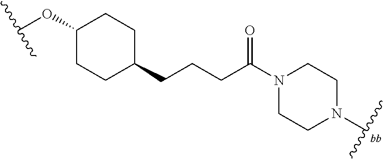

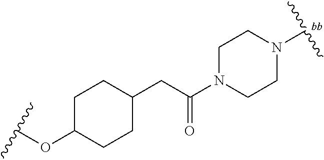

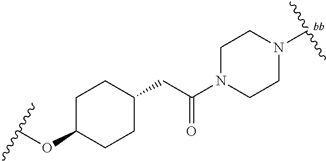

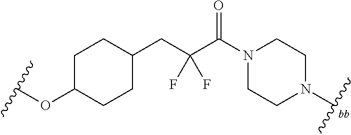

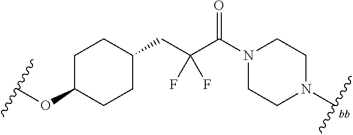

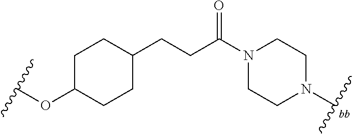

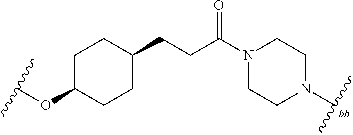

wherein bb represents the point of attachment to Ring C.

In some embodiments of Formula (I-G):
a3 is 1;
L^{A3} is —O—;
a1a is 0; and
L^{A4} is selected from the group consisting of:
C₃₋₁₀ cycloalkylene optionally substituted with 1-3 R^{a}; and
phenylene optionally substituted with 1-3 R^{a}.

In some embodiments of Formula (I-G):
a3 is 1;
L^{A3} is —O—;
a1a is 0;
a1b is 3 or 4;
each L^{A1b} is independently selected from the group consisting of: —CH₂— and —CH(Me)-, provided that 0-1 of L^{A1b} is —CH(Me)-; and
L^{A4} is selected from the group consisting of:

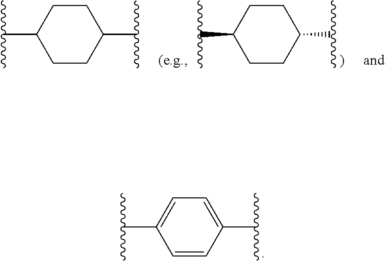 [SMILES: *C1CCC(*)CC1 ; *[C@H]1CC[C@H](*)CC1 ; *c1ccc(*)cc1]

In some embodiments of Formula (I-G):
a3 is 1;
L^{A3} is —O—;
a1a is 2, 3, or 4; and
L^{A4} is 4-10 membered heterocyclylene optionally substituted with 1-3 R^{a}.

In some embodiments of Formula (I-G-1):
a3 is 1;
L^{A3} is —O—;
a1a is 2, 3, or 4;
each L^{A1a} is CH₂;
a1b is 0, 1, or 2;
each L^{A1b} is CH₂; and
L^{A4} is 4-8 membered nitrogen-containing heterocyclylene optionally substituted with 1-3 R^{a}.

In some embodiments of Formula (I-G):
a3 is 1;
L^{A3} is —O—;
a1a is 2, 3, or 4;
each L^{A1a} is CH₂;
a1b is 0, 1, or 2;
each L^{A1b} is CH₂; and
L^{A4} is selected from the group consisting of:

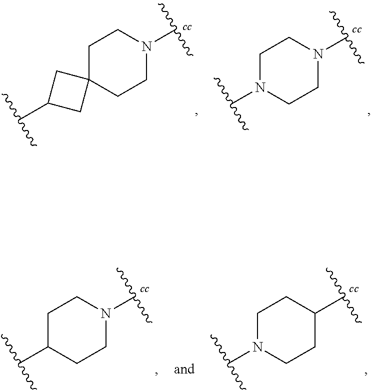

wherein cc represents the point of attachment to L^{A1b}.

In some embodiments of Formula (I-G), the

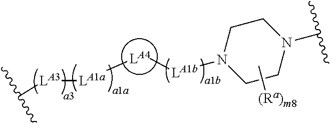

moiety is selected from the groups depicted in Table (L-I-G):

Table (L-I-G)

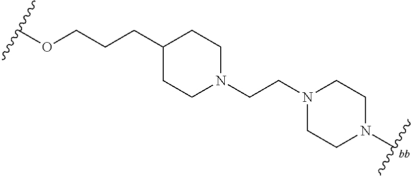

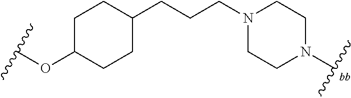

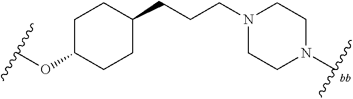

wherein bb represents the point of attachment to Ring C.

In some embodiments, the compounds of Formula (I-H) are compounds of Formula (I-H-1):

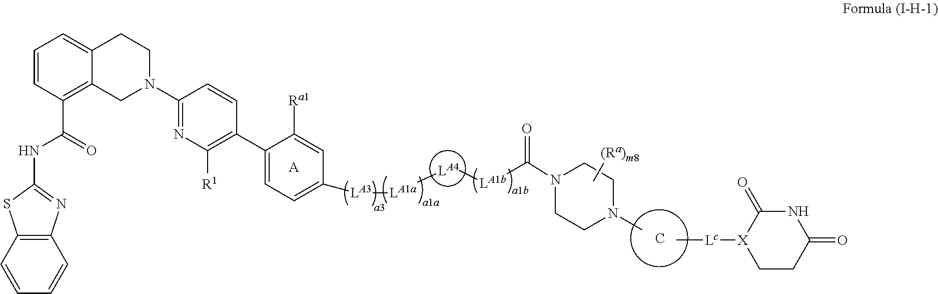

or pharmaceutically acceptable salts thereof, wherein:
R¹ is C(O)OH or C(O)OC₁₋₆ alkyl;
R^{a1} is C₁₋₃ alkyl optionally substituted with 1-3 F;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
m8 is 0, 1, or 2.

In some embodiments, L is (L-4); and the compounds of Formula (I) are compounds of Formula (I-H-2):

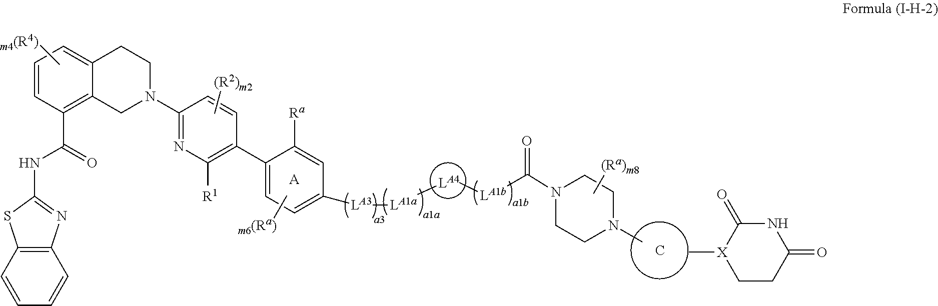

or pharmaceutically acceptable salts thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
m8 is 0, 1, or 2; and
Ring C is

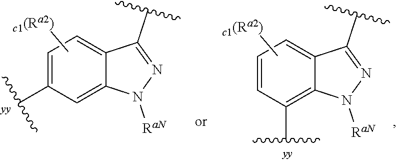

wherein: c1 is 0 or 1; R^{a2} is selected from the group consisting of: halo (e.g., —F) and C₁₋₃ alkyl optionally substituted with 1-3 F; and R^{aN} is C₁₋₃ alkyl.

In some embodiments, L is (L-4); and the compounds of Formula (I) are compounds of Formula (I-H-3):

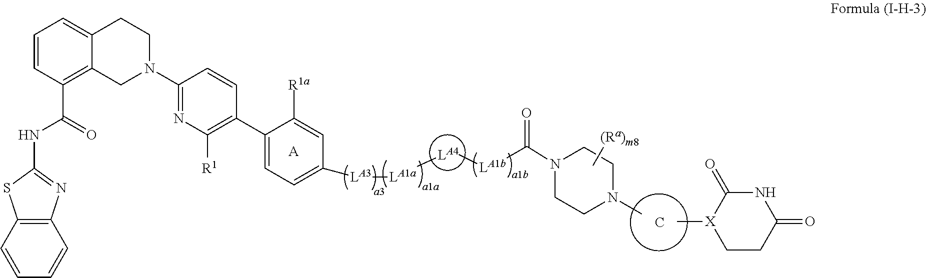

or pharmaceutically acceptable salts thereof, wherein:
R¹ is C(O)OH or C(O)OC₁₋₆ alkyl;
R^{a1} is C₁₋₃ alkyl optionally substituted with 1-3 F;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
m8 is 0, 1, or 2; and
Ring C is

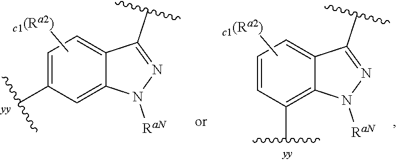

wherein: c1 is 0 or 1; R^{a2} is selected from the group consisting of: halo (e.g., —F) and C₁₋₃ alkyl optionally substituted with 1-3 F; and R^{aN} is C₁₋₃ alkyl.

In some embodiments of Formula (I-H-1), (I-H-2), or (I-H-3):
a3 is 1;
L^{A3} is —O—;
a1a is 0; and
L^{A4} is selected from the group consisting of:
C₃₋₁₀ cycloalkylene optionally substituted with 1-3 R^{a}; and
phenylene optionally substituted with 1-3 R^{a}.

In some embodiments of Formula (I-H-1), (I-H-2), or (I-H-3):
a3 is 1;
L^{A3} is —O—;
a1a is 0;
a1b is 3 or 4;
each L^{A1b} is independently selected from the group consisting of: —CH₂— and —CH(Me)-, provided that 0-1 of L^{A1b} is —CH(Me)-; and
L^{A4} is selected from the group consisting of:

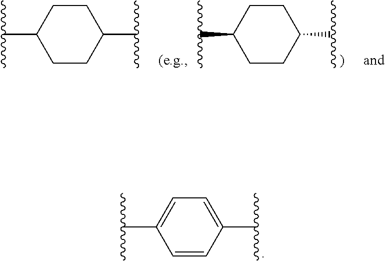 [SMILES: *C1CCC(*)CC1 ; *[C@H]1CC[C@H](*)CC1 ; *c1ccc(*)cc1]

In some embodiments of Formula (I-H-1), (I-H-2), or (I-H-3):
a3 is 1;
L^{A3} is —O—;
a1a is 2, 3, or 4; and
L^{A4} is 4-10 membered heterocyclylene optionally substituted with 1-3 R^{a}.

In some embodiments of Formula (I-H-1), (I-H-2), or (I-H-3):
a3 is 1;
L^{A3} is —O—;
a1a is 2, 3, or 4;
each L^{A1a} is CH₂;
a1b is 0, 1, or 2;
each L^{A1b} is CH₂; and
L^{A4} is 4-8 membered nitrogen-containing heterocyclylene optionally substituted with 1-3 R^{a}.

In some embodiments of Formula (I-H-1), (I-H-2), or (I-H-3), the

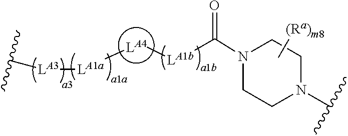

moiety is selected from the groups depicted in Table (L-I-H).

Table (L-I-H)

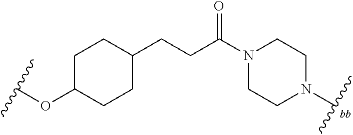

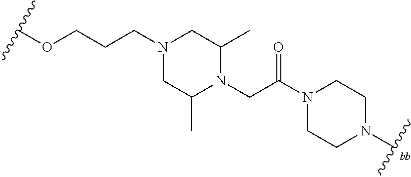

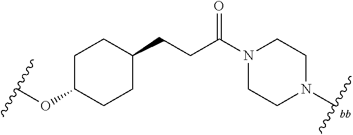

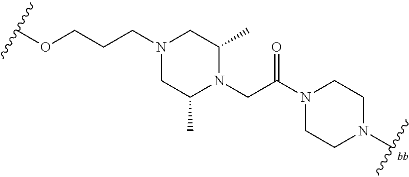

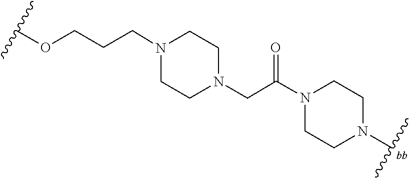

wherein bb represents the point of attachment to Ring C.

In some embodiments, the compounds are selected from the group consisting of the compounds in Table C1, or pharmaceutically acceptable salts thereof.

TABLE C1

| No. | Compound Structure |
|---|---|
| 101 | 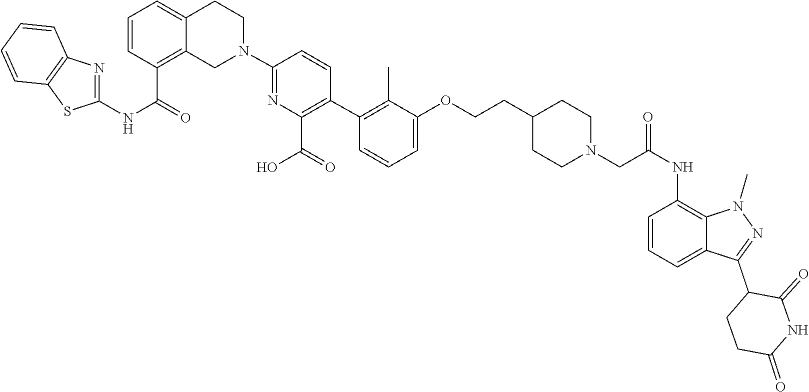 [SMILES: Cc1c(OCCC2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| 102 | 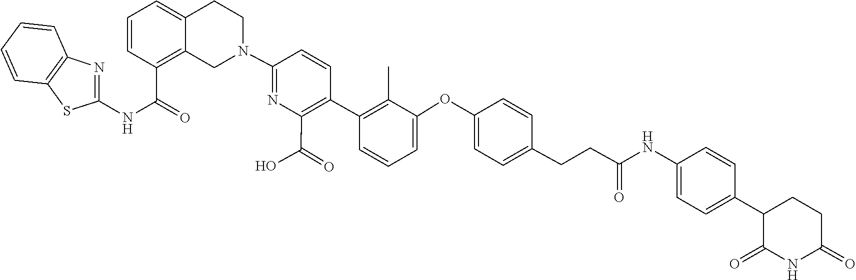 [SMILES: Cc1c(Oc2ccc(CCC(=O)Nc3ccc(C4CCC(=O)NC4=O)cc3)cc2)cccc1-c1ccc(N2CCc3cccc(C(=O)Nc4nc5ccccc5s4)c3C2)nc1C(=O)O] |
| 103 | 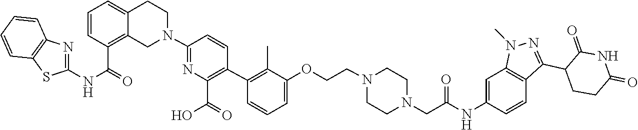 [SMILES: Cc1c(OCCN2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 104 | 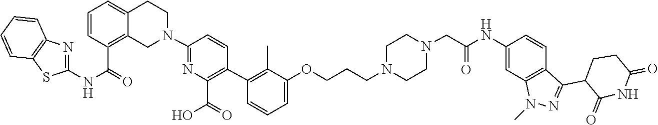 [SMILES: Cc1c(OCCCN2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 105 | 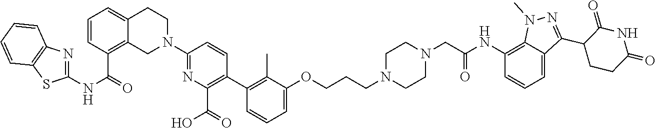 [SMILES: Cc1c(OCCCN2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| 106 | 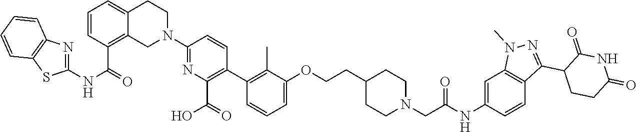 [SMILES: Cc1c(OCCC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 107 | 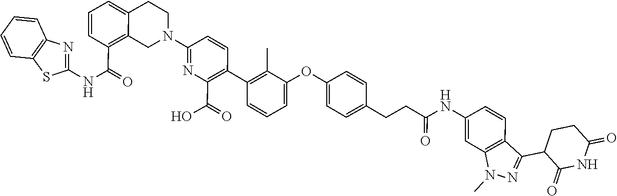 [SMILES: Cc1c(Oc2ccc(CCC(=O)Nc3ccc4c(C5CCC(=O)NC5=O)nn(C)c4c3)cc2)cccc1-c1ccc(N2CCc3cccc(C(=O)Nc4nc5ccccc5s4)c3C2)nc1C(=O)O] |
| 108 | 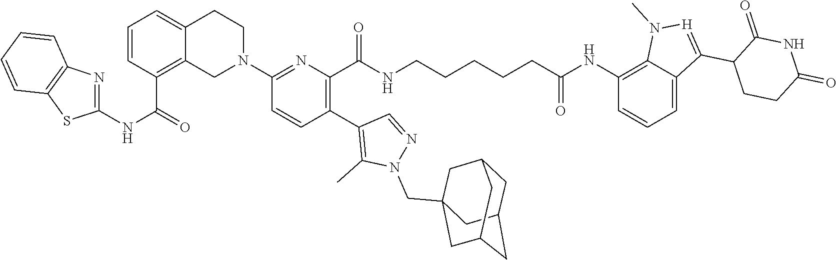 |
| 109 | 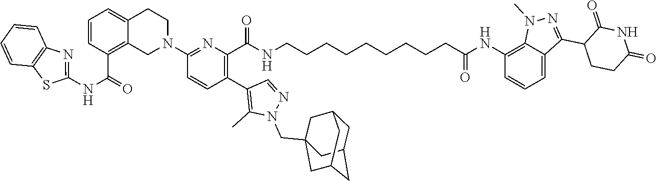 [SMILES: Cc1c(-c2ccc(N3CCc4cccc(C(=O)Nc5nc6ccccc6s5)c4C3)nc2C(=O)NCCCCCCCCCC(=O)Nc2cccc3c(C4CCC(=O)NC4=O)nn(C)c23)cnn1CC12CC3CC(CC(C3)C1)C2] |
| 110 | 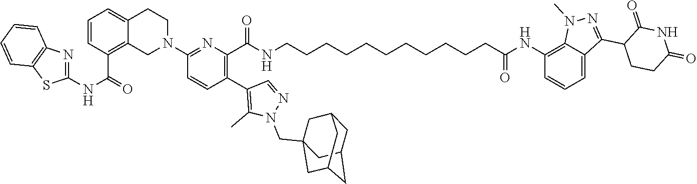 [SMILES: Cc1c(-c2ccc(N3CCc4cccc(C(=O)Nc5nc6ccccc6s5)c4C3)nc2C(=O)NCCCCCCCCCCCC(=O)Nc2cccc3c(C4CCC(=O)NC4=O)nn(C)c23)cnn1CC12CC3CC(CC(C3)C1)C2] |
| 111 | 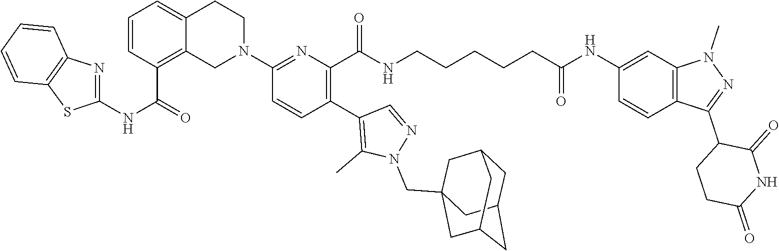 [SMILES: Cc1c(-c2ccc(N3CCc4cccc(C(=O)Nc5nc6ccccc6s5)c4C3)nc2C(=O)NCCCCCC(=O)Nc2ccc3c(C4CCC(=O)NC4=O)nn(C)c3c2)cnn1CC12CC3CC(CC(C3)C1)C2] |
| 112 | 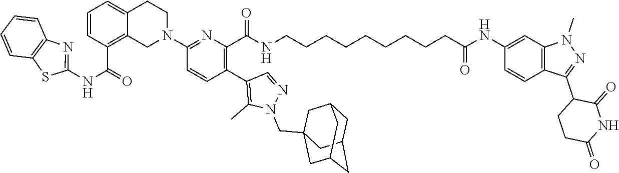 [SMILES: Cc1c(-c2ccc(N3CCc4cccc(C(=O)Nc5nc6ccccc6s5)c4C3)nc2C(=O)NCCCCCCCCCC(=O)Nc2ccc3c(C4CCC(=O)NC4=O)nn(C)c3c2)cnn1CC12CC3CC(CC(C3)C1)C2] |
| 113 | 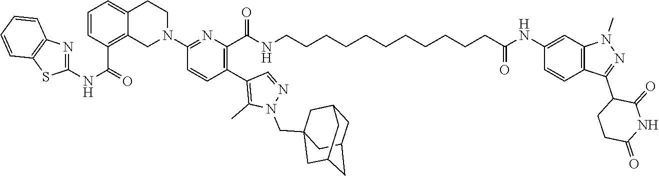 [SMILES: Cc1c(-c2ccc(N3CCc4cccc(C(=O)Nc5nc6ccccc6s5)c4C3)nc2C(=O)NCCCCCCCCCCCC(=O)Nc2ccc3c(C4CCC(=O)NC4=O)nn(C)c3c2)cnn1CC12CC3CC(CC(C3)C1)C2] |
| 114 | 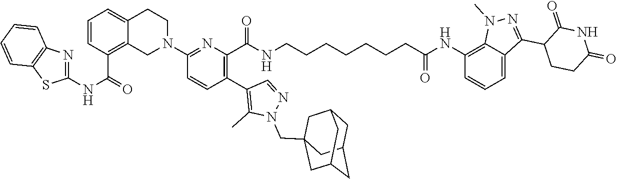 [SMILES: Cc1c(-c2ccc(N3CCc4cccc(C(=O)Nc5nc6ccccc6s5)c4C3)nc2C(=O)NCCCCCCCC(=O)Nc2cccc3c(C4CCC(=O)NC4=O)nn(C)c23)cnn1CC12CC3CC(CC(C3)C1)C2] |
| 115 | 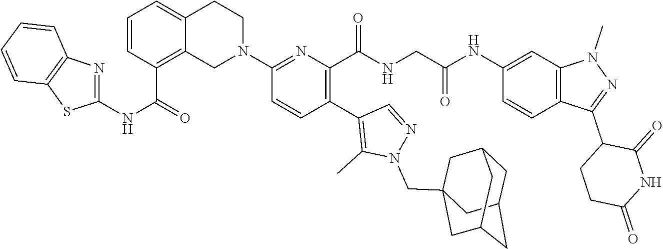 [SMILES: Cc1c(-c2ccc(N3CCc4cccc(C(=O)Nc5nc6ccccc6s5)c4C3)nc2C(=O)NCC(=O)Nc2ccc3c(C4CCC(=O)NC4=O)nn(C)c3c2)cnn1CC12CC3CC(CC(C3)C1)C2] |
| 116 | 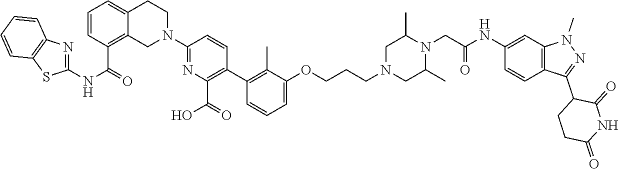 [SMILES: Cc1c(OCCCN2CC(C)N(CC(=O)Nc3ccc4c(C5CCC(=O)NC5=O)nn(C)c4c3)C(C)C2)cccc1-c1ccc(N2CCc3cccc(C(=O)Nc4nc5ccccc5s4)c3C2)nc1C(=O)O] |
| 116a | 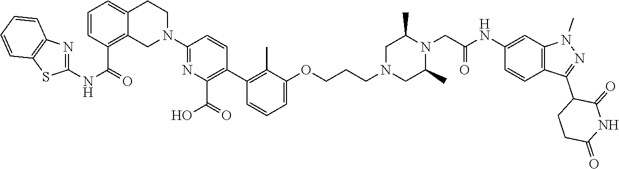 [SMILES: Cc1c(OCCCN2C[C@@H](C)N(CC(=O)Nc3ccc4c(C5CCC(=O)NC5=O)nn(C)c4c3)[C@@H](C)C2)cccc1-c1ccc(N2CCc3cccc(C(=O)Nc4nc5ccccc5s4)c3C2)nc1C(=O)O] |
| 117 | 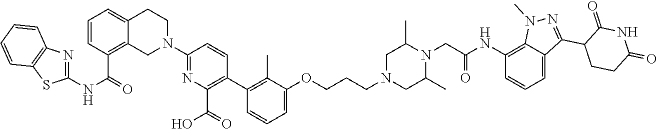 [SMILES: Cc1c(OCCCN2CC(C)N(CC(=O)Nc3cccc4c(C5CCC(=O)NC5=O)nn(C)c34)C(C)C2)cccc1-c1ccc(N2CCc3cccc(C(=O)Nc4nc5ccccc5s4)c3C2)nc1C(=O)O] |
| 117a | 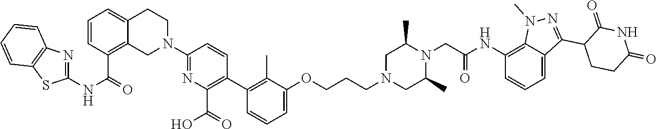 [SMILES: Cc1c(OCCCN2C[C@@H](C)N(CC(=O)Nc3cccc4c(C5CCC(=O)NC5=O)nn(C)c34)[C@@H](C)C2)cccc1-c1ccc(N2CCc3cccc(C(=O)Nc4nc5ccccc5s4)c3C2)nc1C(=O)O] |
| 118 | 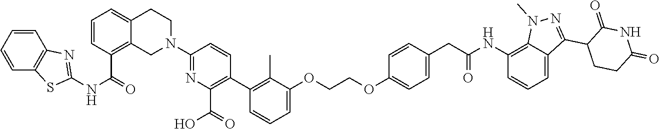 [SMILES: Cc1c(OCCOc2ccc(CC(=O)Nc3cccc4c(C5CCC(=O)NC5=O)nn(C)c34)cc2)cccc1-c1ccc(N2CCc3cccc(C(=O)Nc4nc5ccccc5s4)c3C2)nc1C(=O)O] |
| 119 | 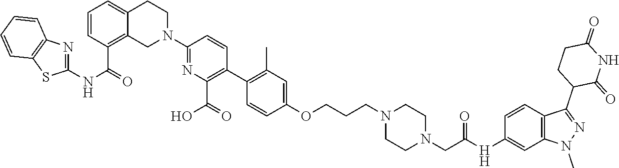 |
| 120 | 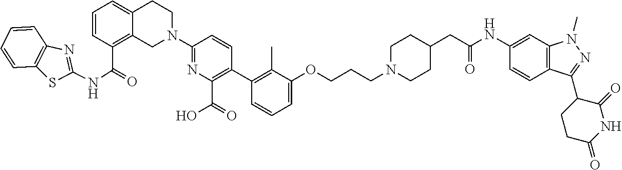 [SMILES: Cc1c(OCCCN2CCC(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 121 | 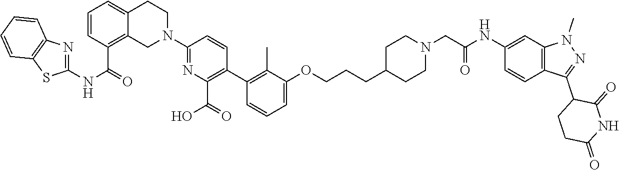 [SMILES: Cc1c(OCCCC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 122 | 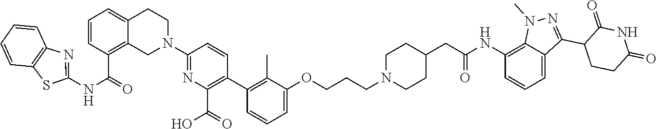 [SMILES: Cc1c(OCCCN2CCC(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| 123 | 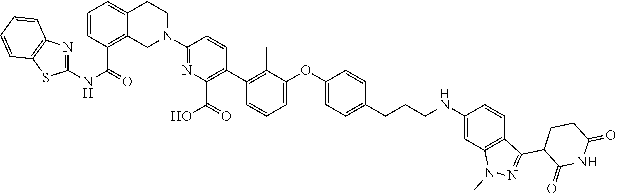 [SMILES: Cc1c(Oc2ccc(CCCNc3ccc4c(C5CCC(=O)NC5=O)nn(C)c4c3)cc2)cccc1-c1ccc(N2CCc3cccc(C(=O)Nc4nc5ccccc5s4)c3C2)nc1C(=O)O] |
| 124 | 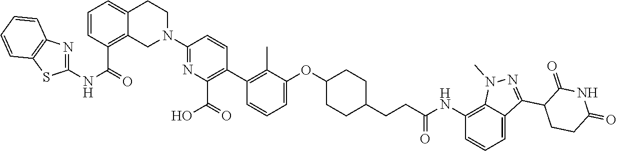 [SMILES: Cc1c(OC2CCC(CCC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| 124a | 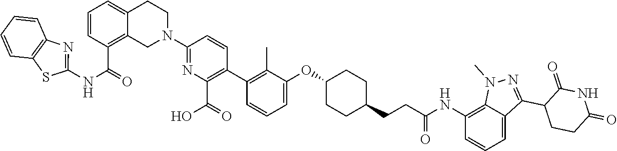 [SMILES: Cc1c(O[C@H]2CC[C@H](CCC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| 125 | 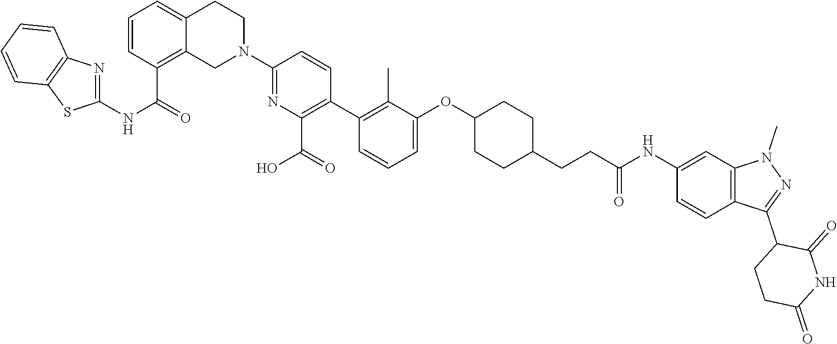 [SMILES: Cc1c(OC2CCC(CCC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 125a | 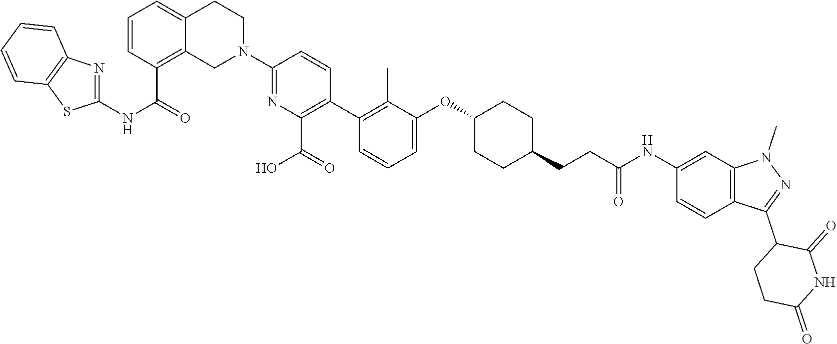 [SMILES: Cc1c(O[C@H]2CC[C@H](CCC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 126 | 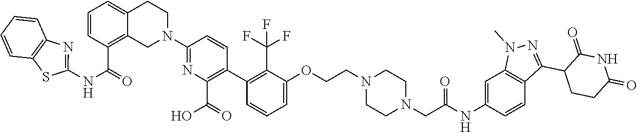 [SMILES: Cn1nc(C2CCC(=O)NC2=O)c2ccc(NC(=O)CN3CCN(CCOc4cccc(-c5ccc(N6CCc7cccc(C(=O)Nc8nc9ccccc9s8)c7C6)nc5C(=O)O)c4C(F)(F)F)CC3)cc21] |
| 127 | 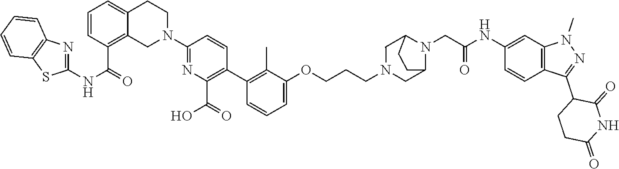 [SMILES: Cc1c(OCCCN2CC3CCC(C2)N3CC(=O)Nc2ccc3c(C4CCC(=O)NC4=O)nn(C)c3c2)cccc1-c1ccc(N2CCc3cccc(C(=O)Nc4nc5ccccc5s4)c3C2)nc1C(=O)O] |
| 128 | 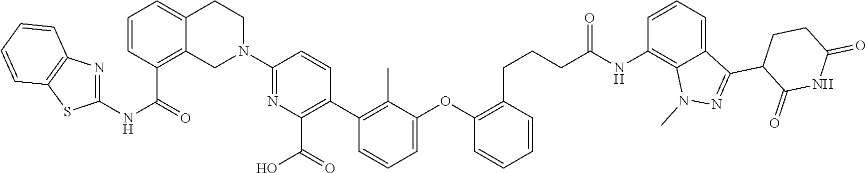 [SMILES: Cc1c(Oc2ccccc2CCCC(=O)Nc2cccc3c(C4CCC(=O)NC4=O)nn(C)c23)cccc1-c1ccc(N2CCc3cccc(C(=O)Nc4nc5ccccc5s4)c3C2)nc1C(=O)O] |
| 129 | 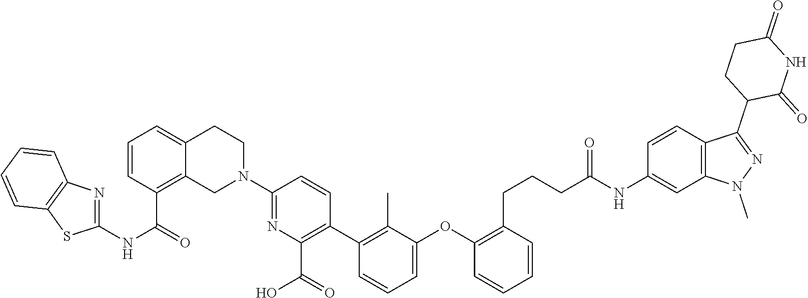 [SMILES: Cc1c(Oc2ccccc2CCCC(=O)Nc2ccc3c(C4CCC(=O)NC4=O)nn(C)c3c2)cccc1-c1ccc(N2CCc3cccc(C(=O)Nc4nc5ccccc5s4)c3C2)nc1C(=O)O] |
| 130 | 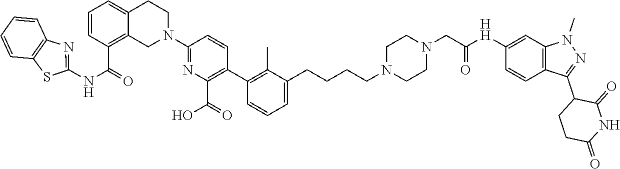 |
| 131 | 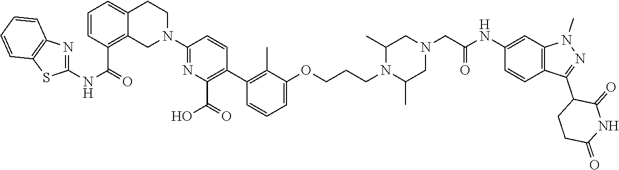 [SMILES: Cc1c(OCCCN2C(C)CN(CC(=O)Nc3ccc4c(C5CCC(=O)NC5=O)nn(C)c4c3)CC2C)cccc1-c1ccc(N2CCc3cccc(C(=O)Nc4nc5ccccc5s4)c3C2)nc1C(=O)O] |
| 131a | 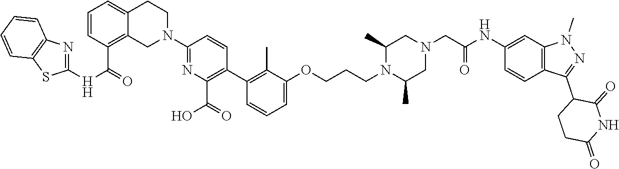 |
| 132 | 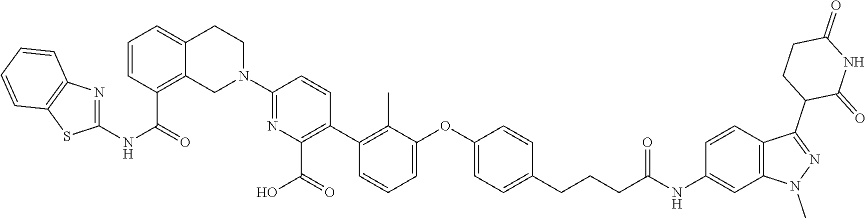 [SMILES: Cc1c(Oc2ccc(CCCC(=O)Nc3ccc4c(C5CCC(=O)NC5=O)nn(C)c4c3)cc2)cccc1-c1ccc(N2CCc3cccc(C(=O)Nc4nc5ccccc5s4)c3C2)nc1C(=O)O] |
| 133 | 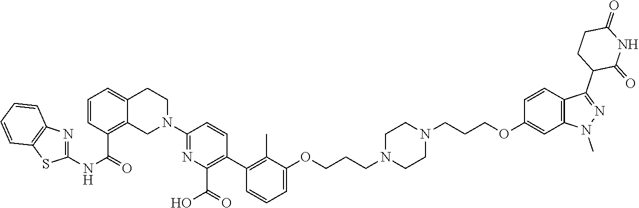 [SMILES: Cc1c(OCCCN2CCN(CCCOc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 134 | 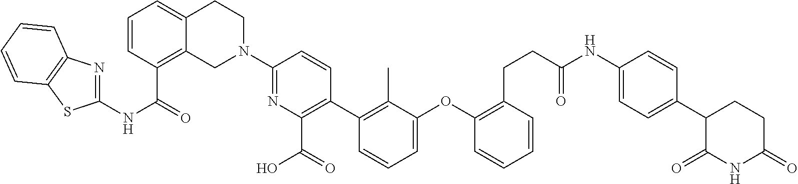 [SMILES: Cc1c(Oc2ccccc2CCC(=O)Nc2ccc(C3CCC(=O)NC3=O)cc2)cccc1-c1ccc(N2CCc3cccc(C(=O)Nc4nc5ccccc5s4)c3C2)nc1C(=O)O] |
| 135 | 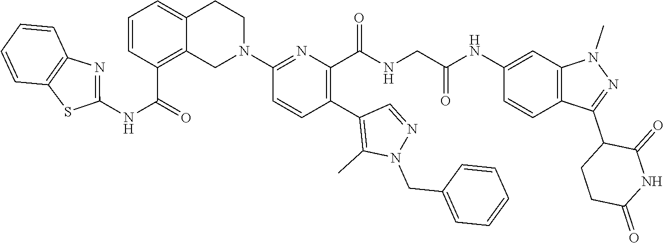 [SMILES: Cc1c(-c2ccc(N3CCc4cccc(C(=O)Nc5nc6ccccc6s5)c4C3)nc2C(=O)NCC(=O)Nc2ccc3c(C4CCC(=O)NC4=O)nn(C)c3c2)cnn1Cc1ccccc1] |
| 136 | 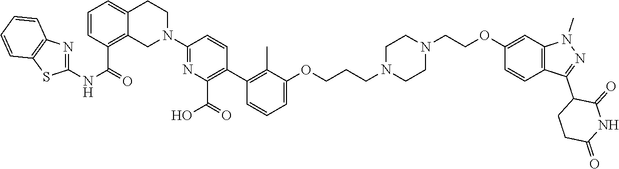 [SMILES: Cc1c(OCCCN2CCN(CCOc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 137 | 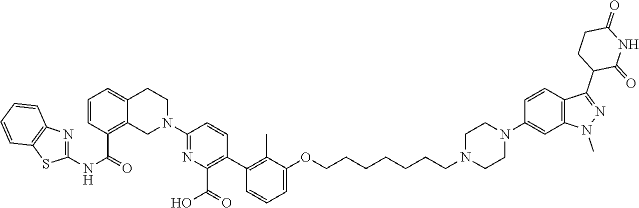 [SMILES: Cc1c(OCCCCCCCN2CCN(c3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 138 | 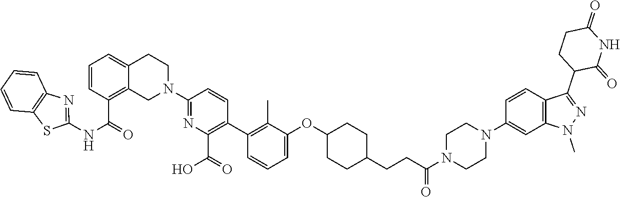 [SMILES: Cc1c(OC2CCC(CCC(=O)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 138a | 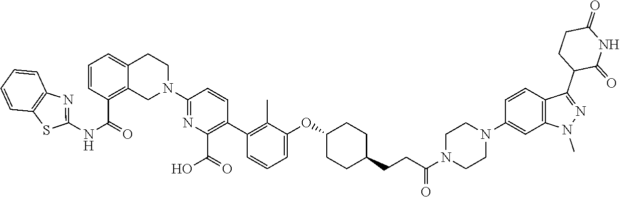 [SMILES: Cc1c(O[C@H]2CC[C@H](CCC(=O)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 138b | 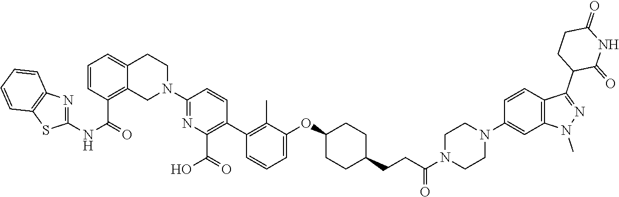 [SMILES: Cc1c(O[C@H]2CC[C@@H](CCC(=O)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 139 | 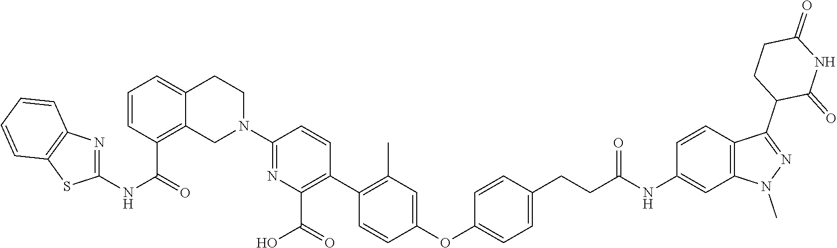 [SMILES: Cc1cc(Oc2ccc(CCC(=O)Nc3ccc4c(C5CCC(=O)NC5=O)nn(C)c4c3)cc2)ccc1-c1ccc(N2CCc3cccc(C(=O)Nc4nc5ccccc5s4)c3C2)nc1C(=O)O] |
| 140 | 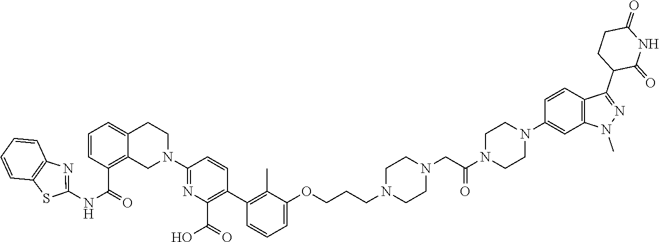 [SMILES: Cc1c(OCCCN2CCN(CC(=O)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 141 | 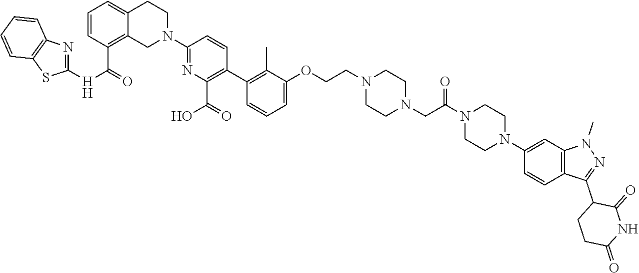 |
| 142 | 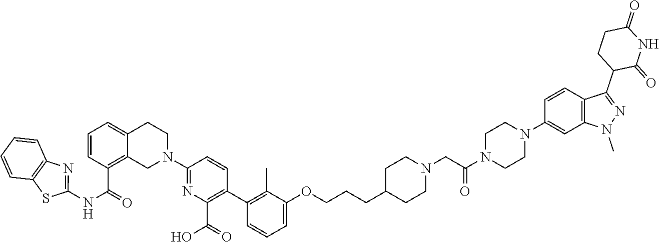 [SMILES: Cc1c(OCCCC2CCN(CC(=O)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 143 | 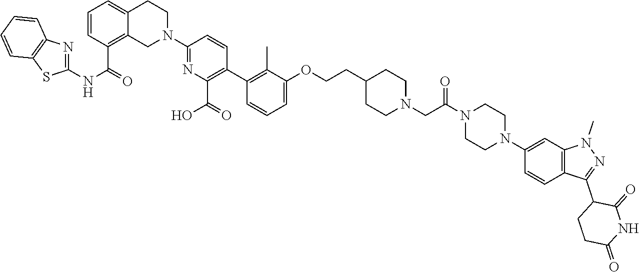 [SMILES: Cc1c(OCCC2CCN(CC(=O)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 144 | 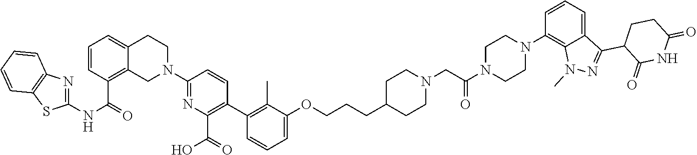 [SMILES: Cc1c(OCCCC2CCN(CC(=O)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 145 | 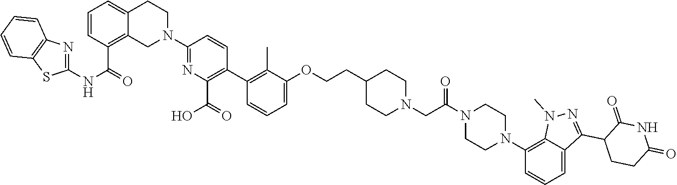 [SMILES: Cc1c(OCCC2CCN(CC(=O)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 146 | 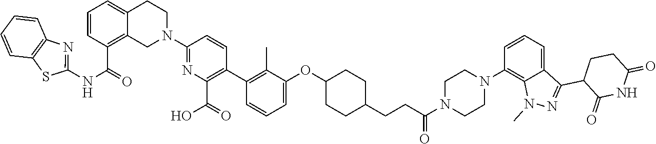 [SMILES: Cc1c(OC2CCC(CCC(=O)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 146a | 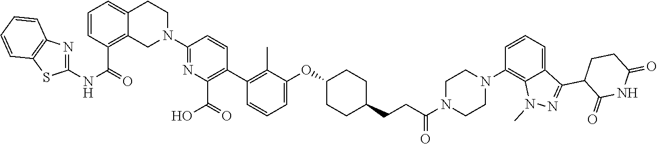 [SMILES: Cc1c(O[C@H]2CC[C@H](CCC(=O)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 146b | 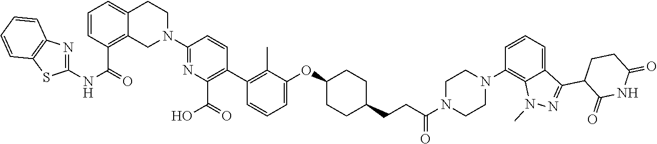 [SMILES: Cc1c(O[C@H]2CC[C@@H](CCC(=O)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 147 | 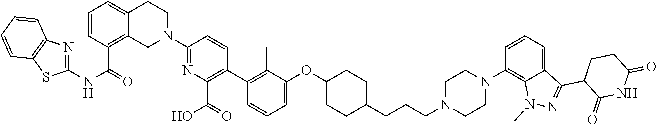 [SMILES: Cc1c(OC2CCC(CCCN3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 147a | 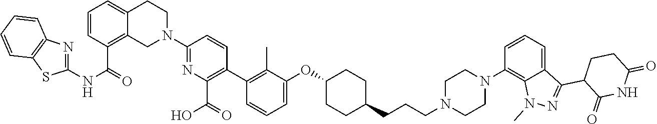 [SMILES: Cc1c(O[C@H]2CC[C@H](CCCN3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 147b | 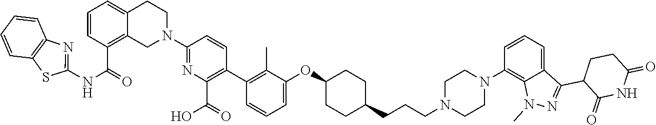 [SMILES: Cc1c(O[C@H]2CC[C@@H](CCCN3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 148 | 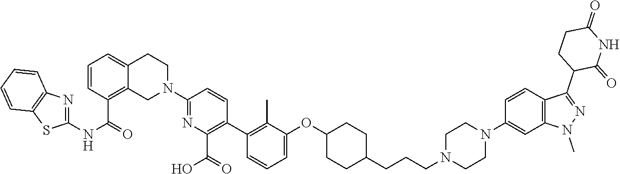 [SMILES: Cc1c(OC2CCC(CCCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 148a | 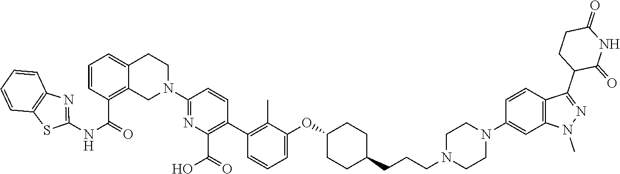 [SMILES: Cc1c(O[C@H]2CC[C@H](CCCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 148b | 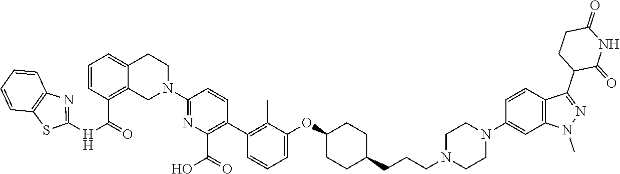 |
| 149 | 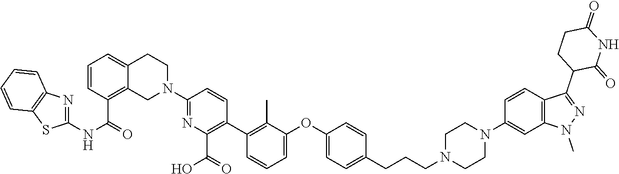 [SMILES: Cc1c(Oc2ccc(CCCN3CCN(c4ccc5c(C6CCC(=O)NC6=O)nn(C)c5c4)CC3)cc2)cccc1-c1ccc(N2CCc3cccc(C(=O)Nc4nc5ccccc5s4)c3C2)nc1C(=O)O] |
| 150 | 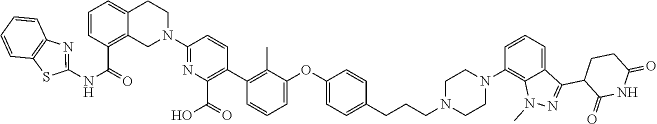 [SMILES: Cc1c(Oc2ccc(CCCN3CCN(c4cccc5c(C6CCC(=O)NC6=O)nn(C)c45)CC3)cc2)cccc1-c1ccc(N2CCc3cccc(C(=O)Nc4nc5ccccc5s4)c3C2)nc1C(=O)O] |
| 151 | 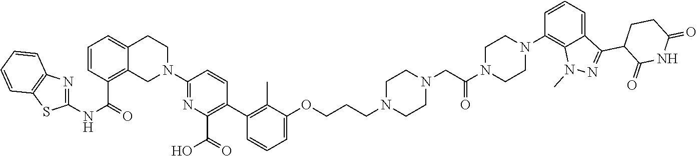 [SMILES: Cc1c(OCCCN2CCN(CC(=O)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 152 | 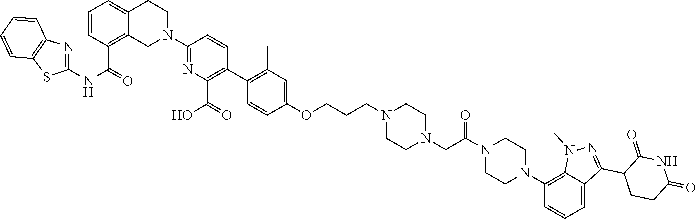 [SMILES: Cc1cc(OCCCN2CCN(CC(=O)N3CCN(c4cccc5c(C6CCC(=O)NC6=O)nn(C)c45)CC3)CC2)ccc1-c1ccc(N2CCc3cccc(C(=O)Nc4nc5ccccc5s4)c3C2)nc1C(=O)O] |
| 153 | 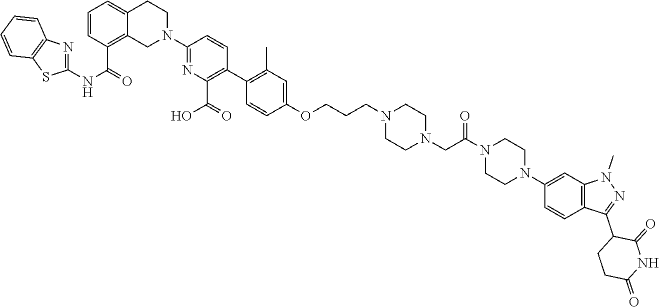 [SMILES: Cc1cc(OCCCN2CCN(CC(=O)N3CCN(c4ccc5c(C6CCC(=O)NC6=O)nn(C)c5c4)CC3)CC2)ccc1-c1ccc(N2CCc3cccc(C(=O)Nc4nc5ccccc5s4)c3C2)nc1C(=O)O] |
| 154 | 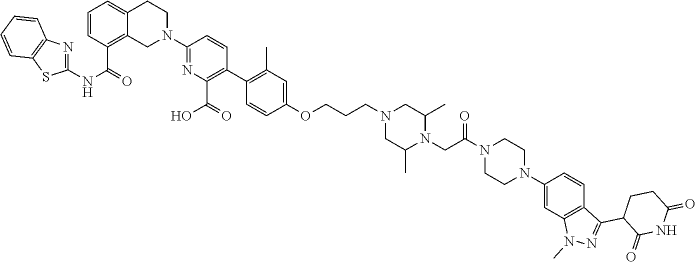 [SMILES: Cc1cc(OCCCN2CC(C)N(CC(=O)N3CCN(c4ccc5c(C6CCC(=O)NC6=O)nn(C)c5c4)CC3)C(C)C2)ccc1-c1ccc(N2CCc3cccc(C(=O)Nc4nc5ccccc5s4)c3C2)nc1C(=O)O] |
| 154a | 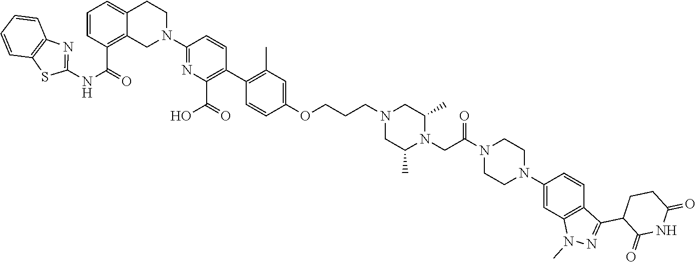 [SMILES: Cc1cc(OCCCN2C[C@@H](C)N(CC(=O)N3CCN(c4ccc5c(C6CCC(=O)NC6=O)nn(C)c5c4)CC3)[C@@H](C)C2)ccc1-c1ccc(N2CCc3cccc(C(=O)Nc4nc5ccccc5s4)c3C2)nc1C(=O)O] |
| 155 | 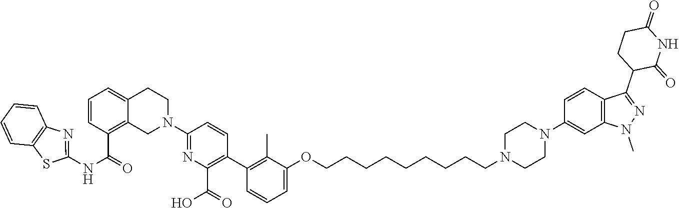 [SMILES: Cc1c(OCCCCCCCCCN2CCN(c3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 156 | 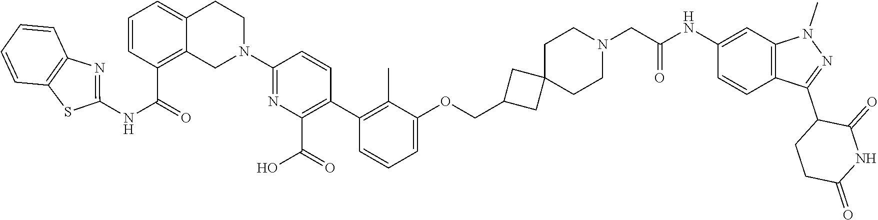 [SMILES: Cc1c(OCC2CC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| 157 | 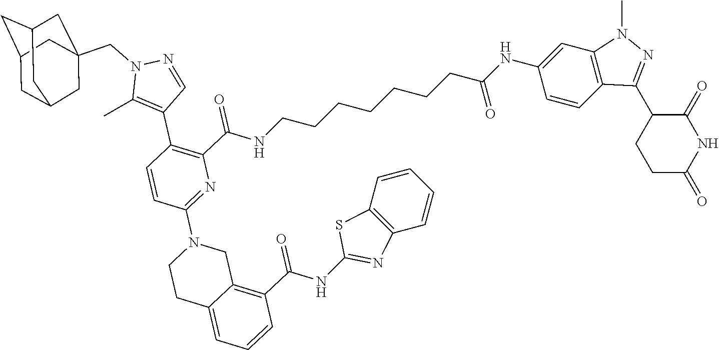 [SMILES: Cc1c(-c2ccc(N3CCc4cccc(C(=O)Nc5nc6ccccc6s5)c4C3)nc2C(=O)NCCCCCCCC(=O)Nc2ccc3c(C4CCC(=O)NC4=O)nn(C)c3c2)cnn1CC12CC3CC(CC(C3)C1)C2] |
| 158 | 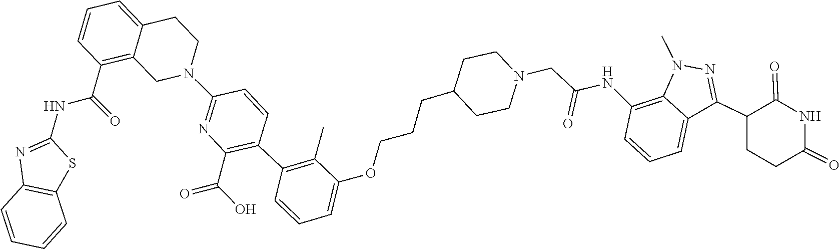 [SMILES: Cc1c(OCCCC2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| 159 | 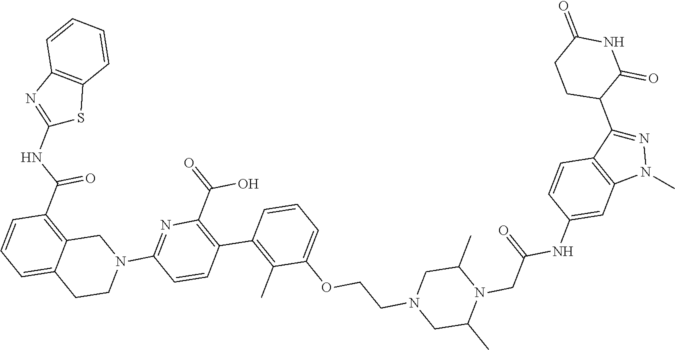 [SMILES: Cc1c(OCCN2CC(C)N(CC(=O)Nc3ccc4c(C5CCC(=O)NC5=O)nn(C)c4c3)C(C)C2)cccc1-c1ccc(N2CCc3cccc(C(=O)Nc4nc5ccccc5s4)c3C2)nc1C(=O)O] |
| 159a | 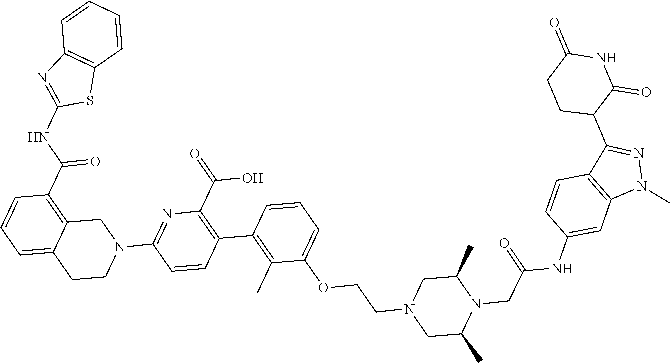 [SMILES: Cc1c(OCCN2C[C@@H](C)N(CC(=O)Nc3ccc4c(C5CCC(=O)NC5=O)nn(C)c4c3)[C@@H](C)C2)cccc1-c1ccc(N2CCc3cccc(C(=O)Nc4nc5ccccc5s4)c3C2)nc1C(=O)O] |
| 160 | 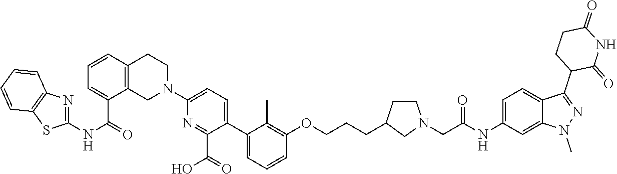 [SMILES: Cc1c(OCCCC2CCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| 160a | 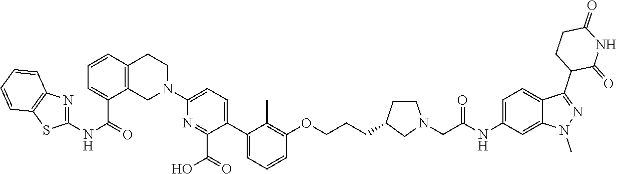 [SMILES: Cc1c(OCCC[C@@H]2CCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| 160b | 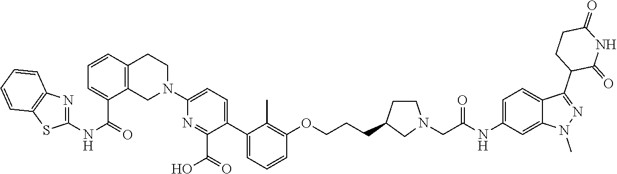 [SMILES: Cc1c(OCCC[C@H]2CCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| 161 | 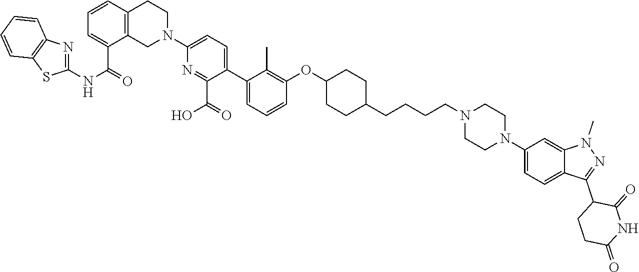 [SMILES: Cc1c(OC2CCC(CCCCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 161a | 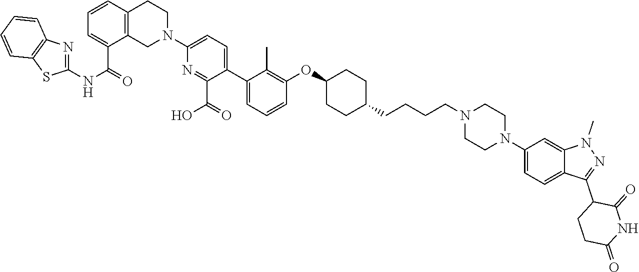 [SMILES: Cc1c(O[C@H]2CC[C@H](CCCCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 162 | 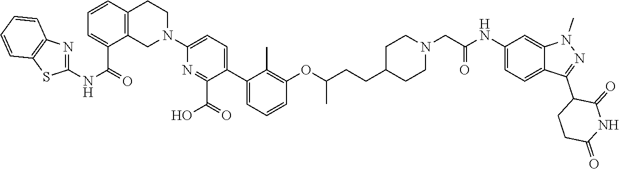 [SMILES: Cc1c(OC(C)CCC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 162a | 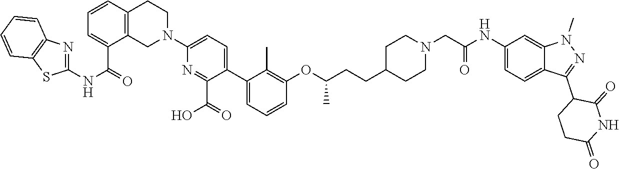 [SMILES: Cc1c(O[C@@H](C)CCC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 162b | 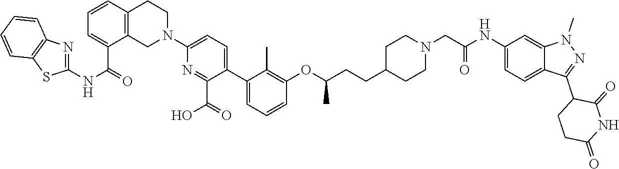 [SMILES: Cc1c(O[C@H](C)CCC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 163 | 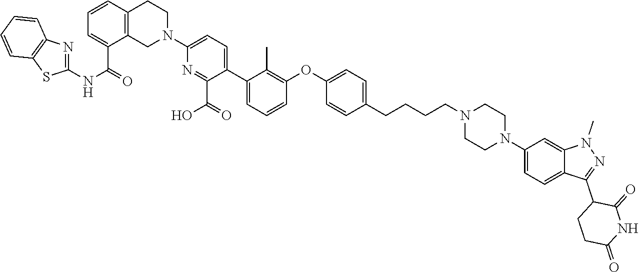 [SMILES: Cc1c(Oc2ccc(CCCCN3CCN(c4ccc5c(C6CCC(=O)NC6=O)nn(C)c5c4)CC3)cc2)cccc1-c1ccc(N2CCc3cccc(C(=O)Nc4nc5ccccc5s4)c3C2)nc1C(=O)O] |
| 164 | 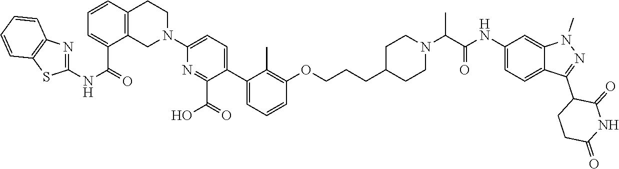 [SMILES: Cc1c(OCCCC2CCN(C(C)C(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 164a | 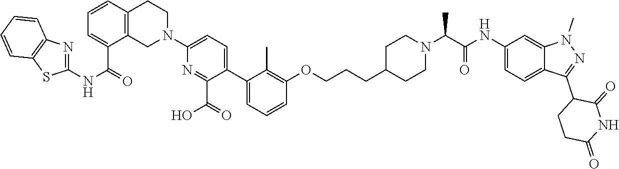 [SMILES: Cc1c(OCCCC2CCN([C@@H](C)C(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 164b | 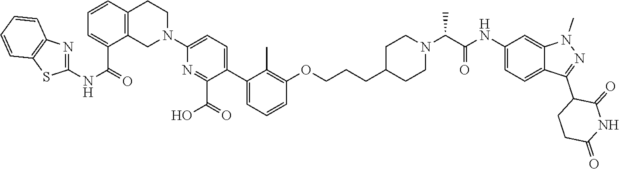 [SMILES: Cc1c(OCCCC2CCN([C@H](C)C(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 165 | 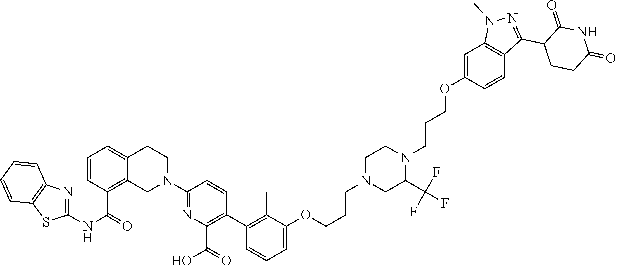 [SMILES: Cc1c(OCCCN2CCN(CCCOc3ccc4c(C5CCC(=O)NC5=O)nn(C)c4c3)C(C(F)(F)F)C2)cccc1-c1ccc(N2CCc3cccc(C(=O)Nc4nc5ccccc5s4)c3C2)nc1C(=O)O] |
| 166 | 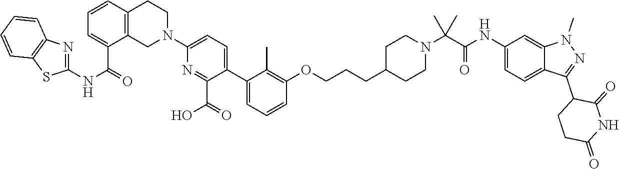 [SMILES: Cc1c(OCCCC2CCN(C(C)(C)C(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 167 | 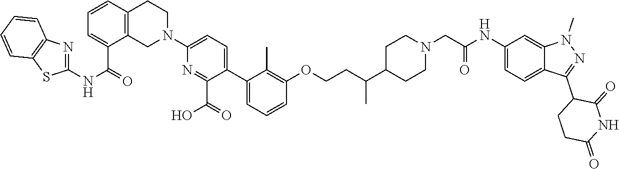 [SMILES: Cc1c(OCCC(C)C2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 167a | 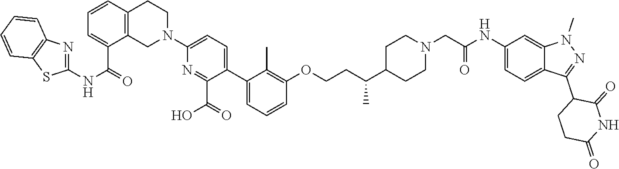 [SMILES: Cc1c(OCC[C@@H](C)C2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 167b | 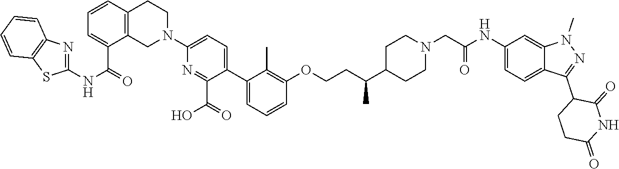 [SMILES: Cc1c(OCC[C@H](C)C2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 168 | 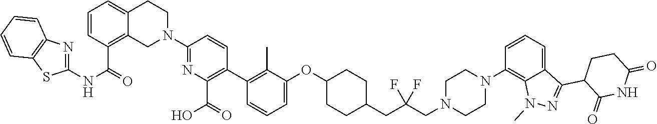 [SMILES: Cc1c(OC2CCC(CC(F)(F)CN3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 168a | 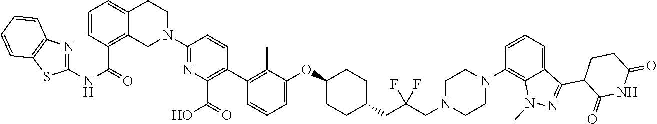 [SMILES: Cc1c(O[C@H]2CC[C@H](CC(F)(F)CN3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 169 | 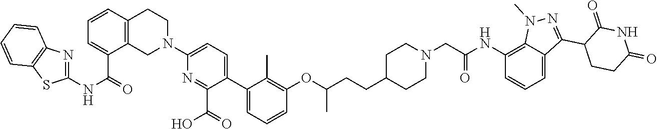 [SMILES: Cc1c(OC(C)CCC2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| 169a | 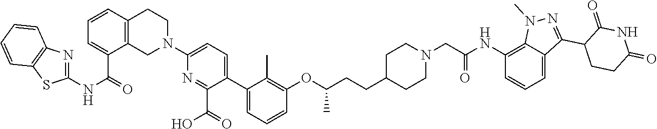 [SMILES: Cc1c(O[C@@H](C)CCC2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| 169b | 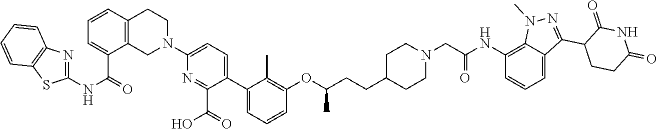 [SMILES: Cc1c(O[C@H](C)CCC2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| 170 | 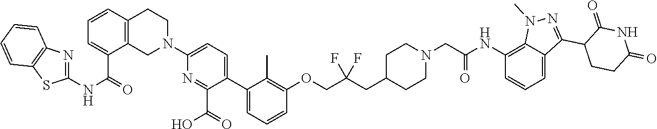 [SMILES: Cc1c(OCC(F)(F)CC2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| 171 | 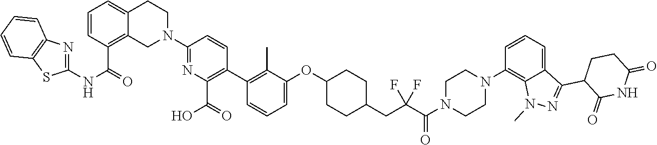 [SMILES: Cc1c(OC2CCC(CC(F)(F)C(=O)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 171a | 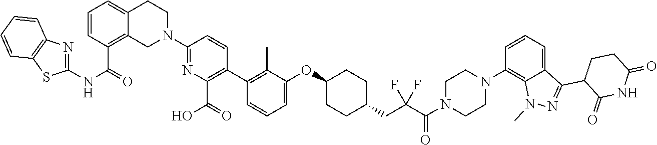 [SMILES: Cc1c(O[C@H]2CC[C@H](CC(F)(F)C(=O)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 172 | 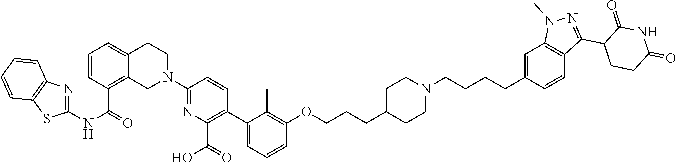 [SMILES: Cc1c(OCCCC2CCN(CCCCc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 173 | 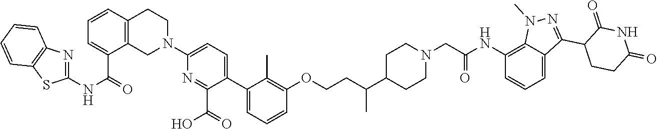 [SMILES: Cc1c(OCCC(C)C2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| 173a | 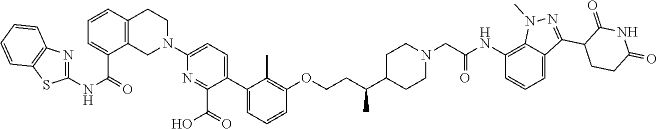 [SMILES: Cc1c(OCC[C@H](C)C2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| 173b | 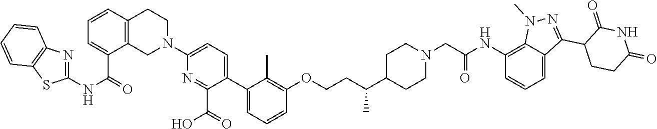 [SMILES: Cc1c(OCC[C@@H](C)C2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| 174 | 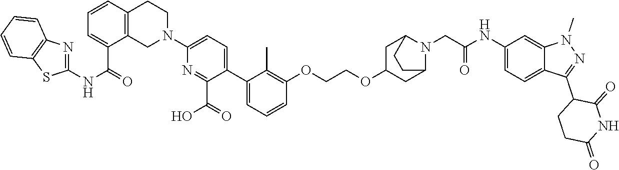 [SMILES: Cc1c(OCCOC2CC3CCC(C2)N3CC(=O)Nc2ccc3c(C4CCC(=O)NC4=O)nn(C)c3c2)cccc1-c1ccc(N2CCc3cccc(C(=O)Nc4nc5ccccc5s4)c3C2)nc1C(=O)O] |
| 174a | 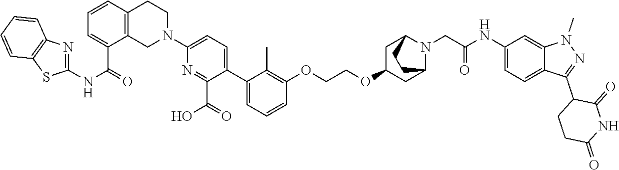 [SMILES: Cc1c(OCCO[C@H]2C[C@H]3CC[C@@H](C2)N3CC(=O)Nc2ccc3c(C4CCC(=O)NC4=O)nn(C)c3c2)cccc1-c1ccc(N2CCc3cccc(C(=O)Nc4nc5ccccc5s4)c3C2)nc1C(=O)O] |
| 174b | 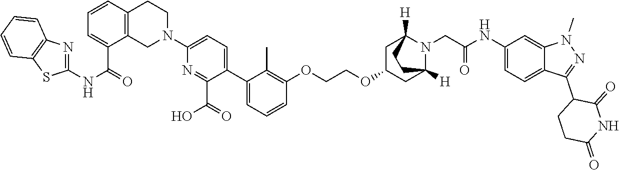 [SMILES: Cc1c(OCCO[C@@H]2C[C@H]3CC[C@@H](C2)N3CC(=O)Nc2ccc3c(C4CCC(=O)NC4=O)nn(C)c3c2)cccc1-c1ccc(N2CCc3cccc(C(=O)Nc4nc5ccccc5s4)c3C2)nc1C(=O)O] |
| 175 | 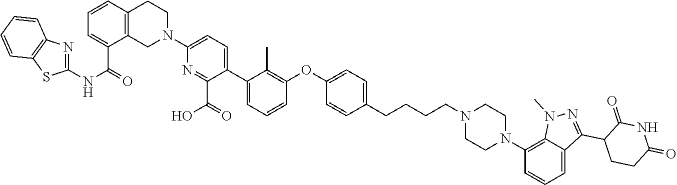 [SMILES: Cc1c(Oc2ccc(CCCCN3CCN(c4cccc5c(C6CCC(=O)NC6=O)nn(C)c45)CC3)cc2)cccc1-c1ccc(N2CCc3cccc(C(=O)Nc4nc5ccccc5s4)c3C2)nc1C(=O)O] |
| 176 | 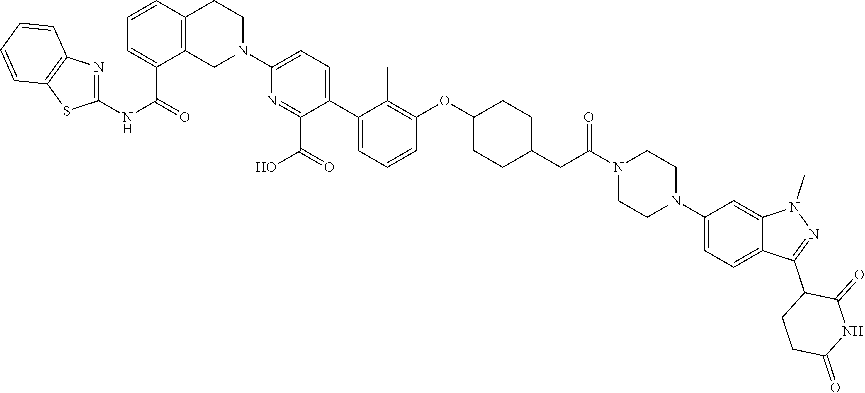 [SMILES: Cc1c(OC2CCC(CC(=O)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 176a | 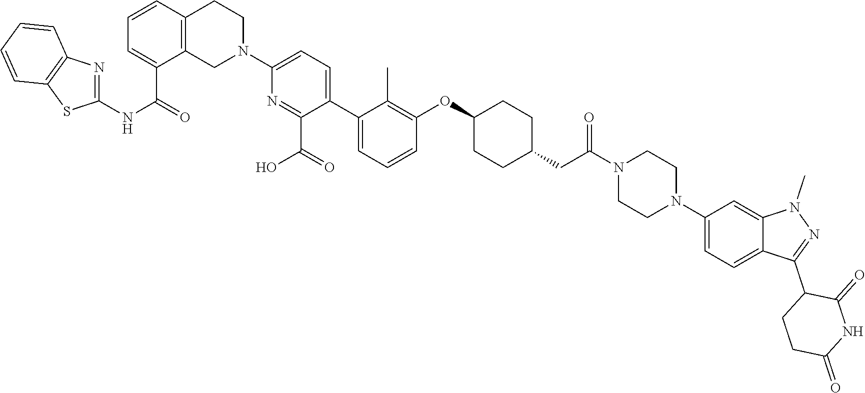 [SMILES: Cc1c(O[C@H]2CC[C@H](CC(=O)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 177 | 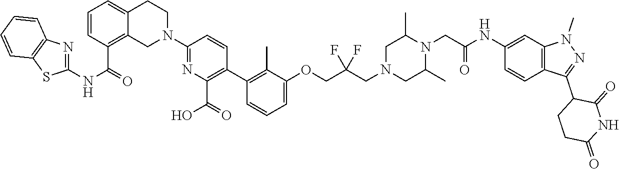 [SMILES: Cc1c(OCC(F)(F)CN2CC(C)N(CC(=O)Nc3ccc4c(C5CCC(=O)NC5=O)nn(C)c4c3)C(C)C2)cccc1-c1ccc(N2CCc3cccc(C(=O)Nc4nc5ccccc5s4)c3C2)nc1C(=O)O] |
| 177a | 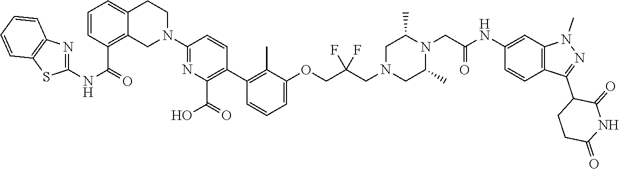 [SMILES: Cc1c(OCC(F)(F)CN2C[C@@H](C)N(CC(=O)Nc3ccc4c(C5CCC(=O)NC5=O)nn(C)c4c3)[C@@H](C)C2)cccc1-c1ccc(N2CCc3cccc(C(=O)Nc4nc5ccccc5s4)c3C2)nc1C(=O)O] |
| 178 | 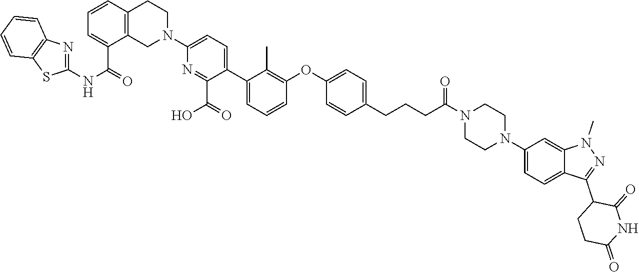 [SMILES: Cc1c(Oc2ccc(CCCC(=O)N3CCN(c4ccc5c(C6CCC(=O)NC6=O)nn(C)c5c4)CC3)cc2)cccc1-c1ccc(N2CCc3cccc(C(=O)Nc4nc5ccccc5s4)c3C2)nc1C(=O)O] |
| 179 | 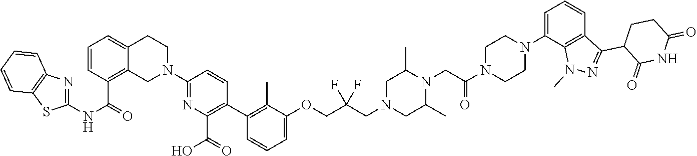 [SMILES: Cc1c(OCC(F)(F)CN2CC(C)N(CC(=O)N3CCN(c4cccc5c(C6CCC(=O)NC6=O)nn(C)c45)CC3)C(C)C2)cccc1-c1ccc(N2CCc3cccc(C(=O)Nc4nc5ccccc5s4)c3C2)nc1C(=O)O] |
| 179a | 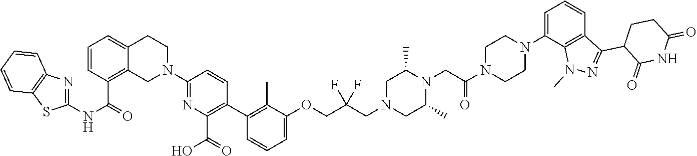 [SMILES: Cc1c(OCC(F)(F)CN2C[C@@H](C)N(CC(=O)N3CCN(c4cccc5c(C6CCC(=O)NC6=O)nn(C)c45)CC3)[C@@H](C)C2)cccc1-c1ccc(N2CCc3cccc(C(=O)Nc4nc5ccccc5s4)c3C2)nc1C(=O)O] |
| 180 | 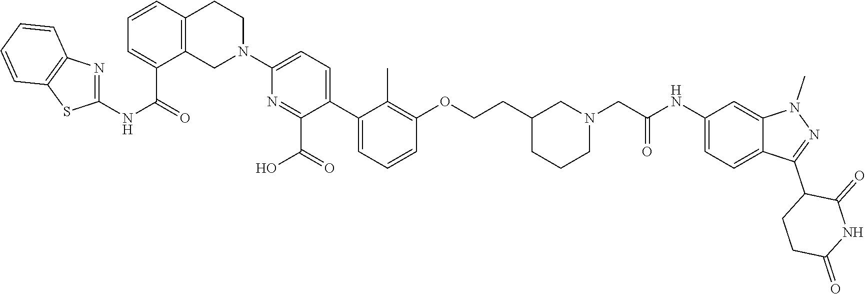 [SMILES: Cc1c(OCCC2CCCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| 180a | 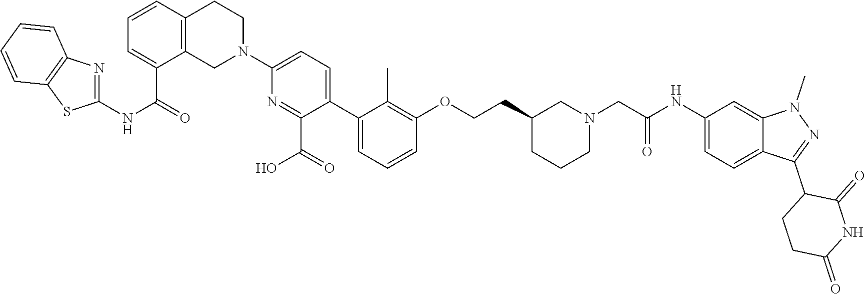 [SMILES: Cc1c(OCC[C@@H]2CCCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| 180b | 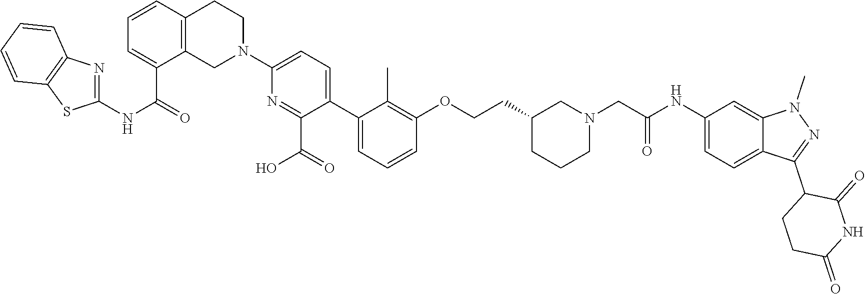 [SMILES: Cc1c(OCC[C@H]2CCCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| 181 | 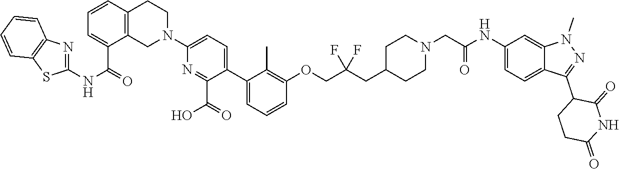 [SMILES: Cc1c(OCC(F)(F)CC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 182 | 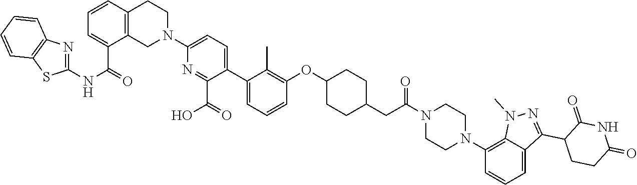 [SMILES: Cc1c(OC2CCC(CC(=O)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 182a | 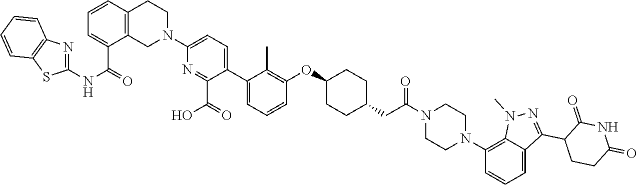 [SMILES: Cc1c(O[C@H]2CC[C@H](CC(=O)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 183 | 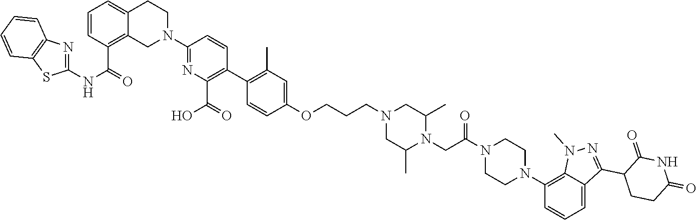 [SMILES: Cc1cc(OCCCN2CC(C)N(CC(=O)N3CCN(c4cccc5c(C6CCC(=O)NC6=O)nn(C)c45)CC3)C(C)C2)ccc1-c1ccc(N2CCc3cccc(C(=O)Nc4nc5ccccc5s4)c3C2)nc1C(=O)O] |
| 183a | 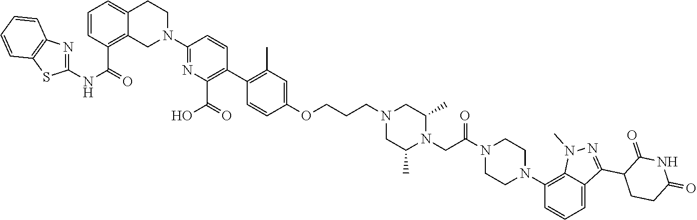 [SMILES: Cc1cc(OCCCN2C[C@@H](C)N(CC(=O)N3CCN(c4cccc5c(C6CCC(=O)NC6=O)nn(C)c45)CC3)[C@@H](C)C2)ccc1-c1ccc(N2CCc3cccc(C(=O)Nc4nc5ccccc5s4)c3C2)nc1C(=O)O] |
| 184 | 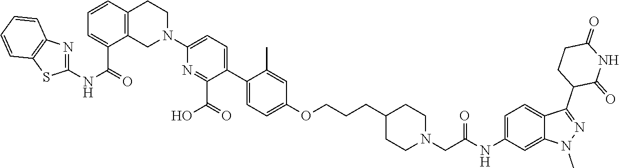 [SMILES: Cc1cc(OCCCC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 185 | 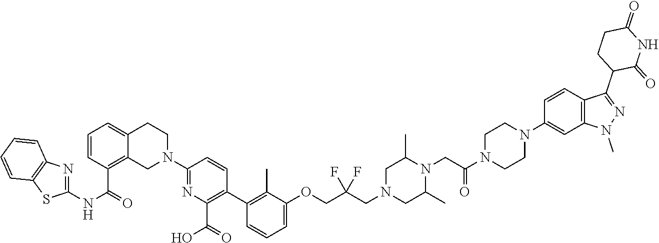 [SMILES: Cc1c(OCC(F)(F)CN2CC(C)N(CC(=O)N3CCN(c4ccc5c(C6CCC(=O)NC6=O)nn(C)c5c4)CC3)C(C)C2)cccc1-c1ccc(N2CCc3cccc(C(=O)Nc4nc5ccccc5s4)c3C2)nc1C(=O)O] |
| 185a | 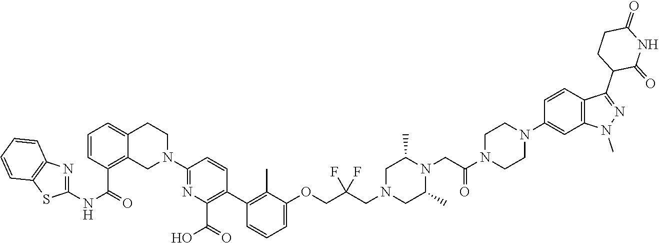 [SMILES: Cc1c(OCC(F)(F)CN2C[C@@H](C)N(CC(=O)N3CCN(c4ccc5c(C6CCC(=O)NC6=O)nn(C)c5c4)CC3)[C@@H](C)C2)cccc1-c1ccc(N2CCc3cccc(C(=O)Nc4nc5ccccc5s4)c3C2)nc1C(=O)O] |
| 186 | 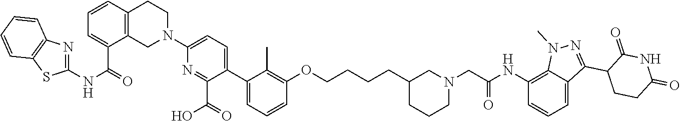 [SMILES: Cc1c(OCCCCC2CCCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O] |
| 186a | 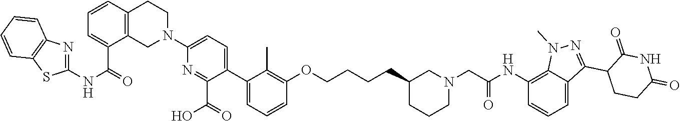 [SMILES: Cc1c(OCCCC[C@@H]2CCCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O] |
| 186b | 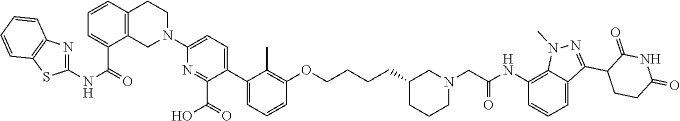 [SMILES: Cc1c(OCCCC[C@H]2CCCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O] |
| 187 | 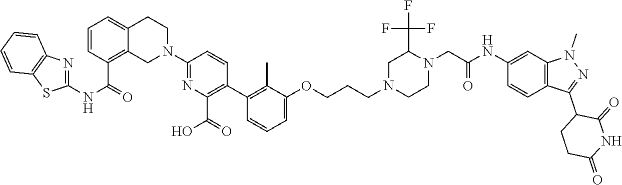 [SMILES: Cc1c(OCCCN2CCN(CC(=O)Nc3ccc4c(C5CCC(=O)NC5=O)nn(C)c4c3)C(C(F)(F)F)C2)cccc1-c1ccc(N2CCc3cccc(C(=O)Nc4nc5ccccc5s4)c3C2)nc1C(=O)O] |
| 187a | 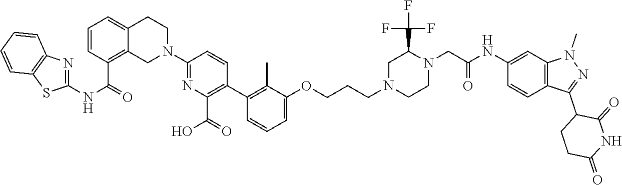 [SMILES: Cc1c(OCCCN2CCN(CC(=O)Nc3ccc4c(C5CCC(=O)NC5=O)nn(C)c4c3)[C@H](C(F)(F)F)C2)cccc1-c1ccc(N2CCc3cccc(C(=O)Nc4nc5ccccc5s4)c3C2)nc1C(=O)O] |
| 187b | 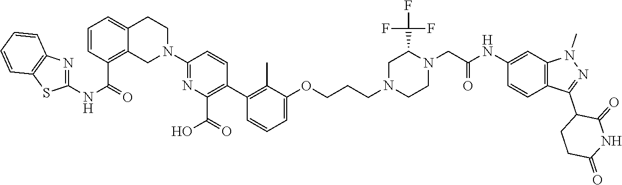 [SMILES: Cc1c(OCCCN2CCN(CC(=O)Nc3ccc4c(C5CCC(=O)NC5=O)nn(C)c4c3)[C@@H](C(F)(F)F)C2)cccc1-c1ccc(N2CCc3cccc(C(=O)Nc4nc5ccccc5s4)c3C2)nc1C(=O)O] |
| 188 | 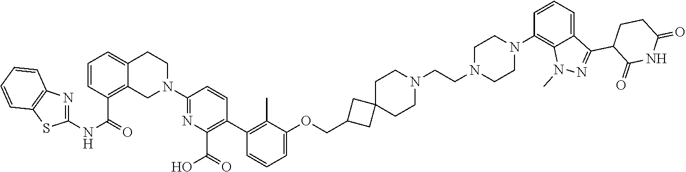 [SMILES: Cc1c(OCC2CC3(CCN(CCN4CCN(c5cccc6c(C7CCC(=O)NC7=O)nn(C)c56)CC4)CC3)C2)cccc1-c1ccc(N2CCc3cccc(C(=O)Nc4nc5ccccc5s4)c3C2)nc1C(=O)O] |
| 189 | 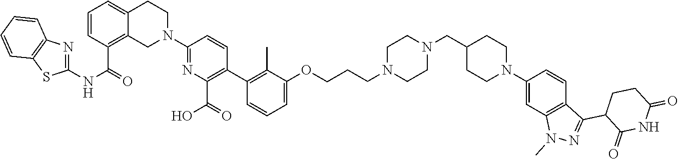 [SMILES: Cc1c(OCCCN2CCN(CC3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 190 | 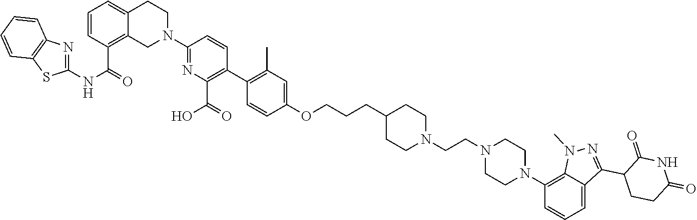 [SMILES: Cc1cc(OCCCC2CCN(CCN3CCN(c4cccc5c(C6CCC(=O)NC6=O)nn(C)c45)CC3)CC2)ccc1-c1ccc(N2CCc3cccc(C(=O)Nc4nc5ccccc5s4)c3C2)nc1C(=O)O] |
| 191 | 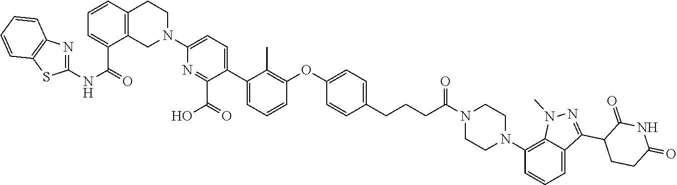 [SMILES: Cc1c(Oc2ccc(CCCC(=O)N3CCN(c4cccc5c(C6CCC(=O)NC6=O)nn(C)c45)CC3)cc2)cccc1-c1ccc(N2CCc3cccc(C(=O)Nc4nc5ccccc5s4)c3C2)nc1C(=O)O] |
| 192 | 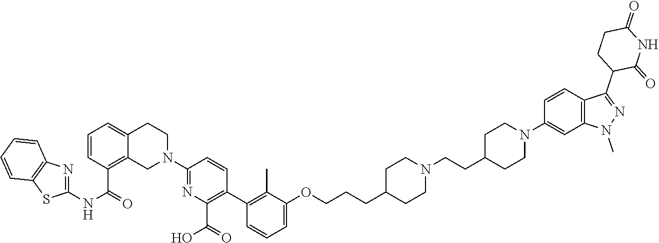 [SMILES: Cc1c(OCCCC2CCN(CCC3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 193 | 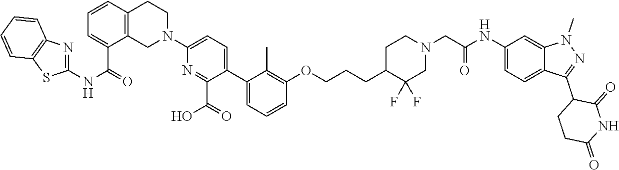 [SMILES: Cc1c(OCCCC2CCN(CC(=O)Nc3ccc4c(C5CCC(=O)NC5=O)nn(C)c4c3)CC2(F)F)cccc1-c1ccc(N2CCc3cccc(C(=O)Nc4nc5ccccc5s4)c3C2)nc1C(=O)O] |
| 193a | 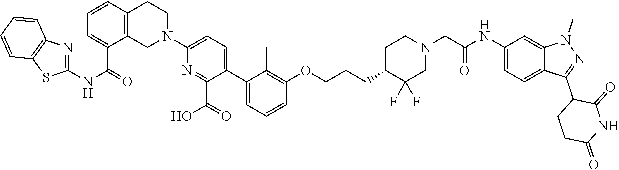 [SMILES: Cc1c(OCCC[C@@H]2CCN(CC(=O)Nc3ccc4c(C5CCC(=O)NC5=O)nn(C)c4c3)CC2(F)F)cccc1-c1ccc(N2CCc3cccc(C(=O)Nc4nc5ccccc5s4)c3C2)nc1C(=O)O] |
| 193b | 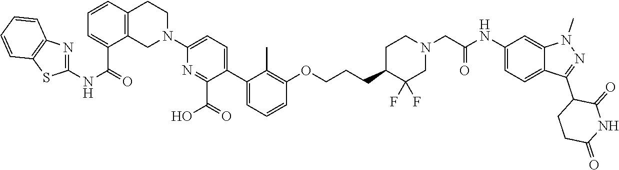 [SMILES: Cc1c(OCCC[C@H]2CCN(CC(=O)Nc3ccc4c(C5CCC(=O)NC5=O)nn(C)c4c3)CC2(F)F)cccc1-c1ccc(N2CCc3cccc(C(=O)Nc4nc5ccccc5s4)c3C2)nc1C(=O)O] |
| 194 | 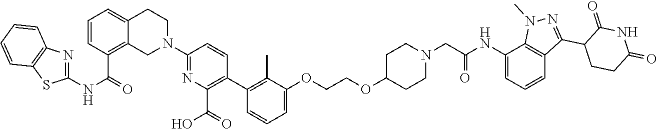 [SMILES: Cc1c(OCCOC2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| 195 | 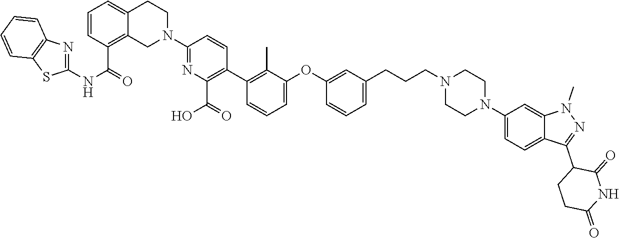 [SMILES: Cc1c(Oc2cccc(CCCN3CCN(c4ccc5c(C6CCC(=O)NC6=O)nn(C)c5c4)CC3)c2)cccc1-c1ccc(N2CCc3cccc(C(=O)Nc4nc5ccccc5s4)c3C2)nc1C(=O)O] |
| 196 | 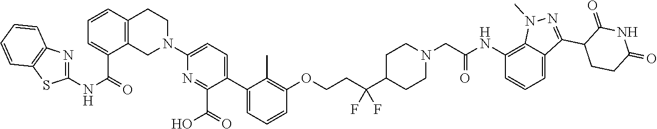 [SMILES: Cc1c(OCCC(F)(F)C2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| 197 | 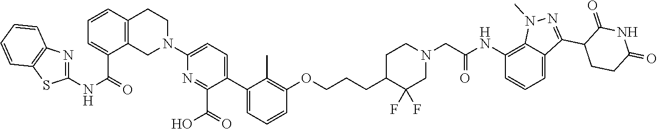 [SMILES: Cc1c(OCCCC2CCN(CC(=O)Nc3cccc4c(C5CCC(=O)NC5=O)nn(C)c34)CC2(F)F)cccc1-c1ccc(N2CCc3cccc(C(=O)Nc4nc5ccccc5s4)c3C2)nc1C(=O)O] |
| 197a | 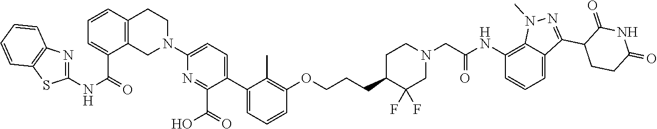 [SMILES: Cc1c(OCCC[C@H]2CCN(CC(=O)Nc3cccc4c(C5CCC(=O)NC5=O)nn(C)c34)CC2(F)F)cccc1-c1ccc(N2CCc3cccc(C(=O)Nc4nc5ccccc5s4)c3C2)nc1C(=O)O] |
| 197b | 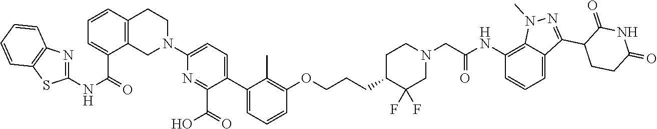 [SMILES: Cc1c(OCCC[C@@H]2CCN(CC(=O)Nc3cccc4c(C5CCC(=O)NC5=O)nn(C)c34)CC2(F)F)cccc1-c1ccc(N2CCc3cccc(C(=O)Nc4nc5ccccc5s4)c3C2)nc1C(=O)O] |
| 198 | 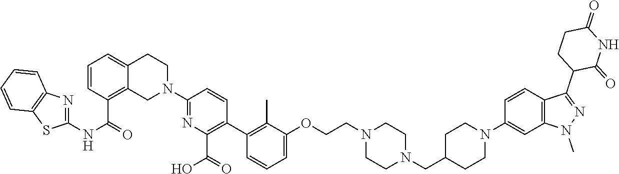 [SMILES: Cc1c(OCCN2CCN(CC3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 199 | 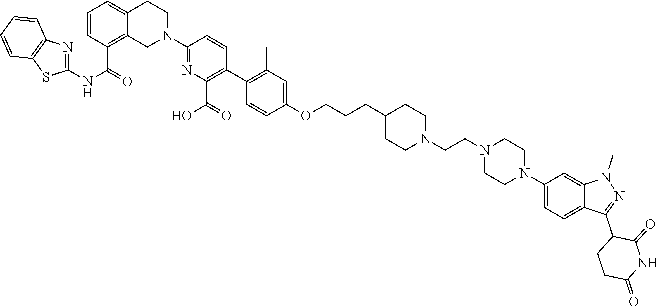 [SMILES: Cc1cc(OCCCC2CCN(CCN3CCN(c4ccc5c(C6CCC(=O)NC6=O)nn(C)c5c4)CC3)CC2)ccc1-c1ccc(N2CCc3cccc(C(=O)Nc4nc5ccccc5s4)c3C2)nc1C(=O)O] |
| 200 | 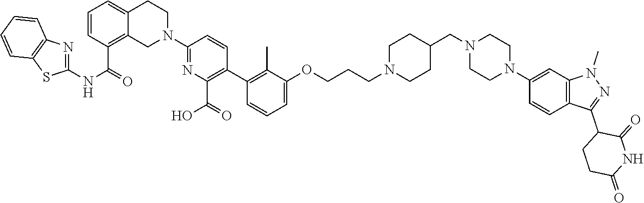 [SMILES: Cc1c(OCCCN2CCC(CN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 201 | 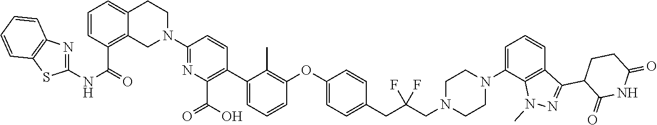 [SMILES: Cc1c(Oc2ccc(CC(F)(F)CN3CCN(c4cccc5c(C6CCC(=O)NC6=O)nn(C)c45)CC3)cc2)cccc1-c1ccc(N2CCc3cccc(C(=O)Nc4nc5ccccc5s4)c3C2)nc1C(=O)O] |
| 202 | 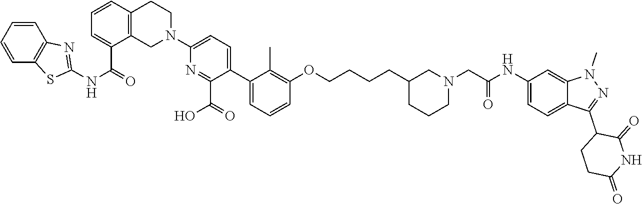 [SMILES: Cc1c(OCCCCC2CCCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| 202a | 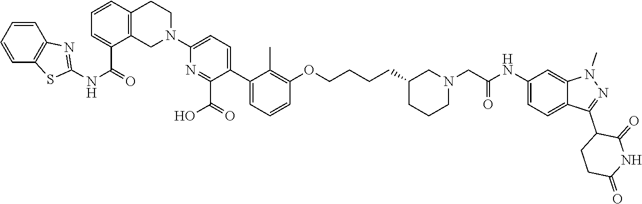 [SMILES: Cc1c(OCCCC[C@H]2CCCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| 202b | 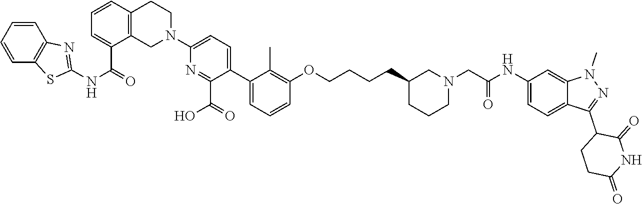 [SMILES: Cc1c(OCCCC[C@@H]2CCCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| 203 | 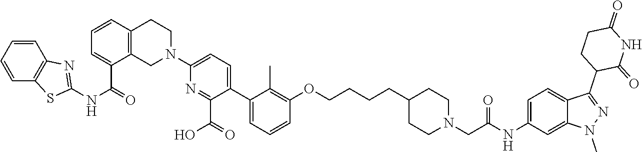 [SMILES: Cc1c(OCCCCC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 204 | 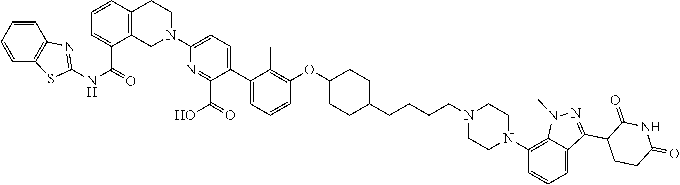 [SMILES: Cc1c(OC2CCC(CCCCN3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 204a | 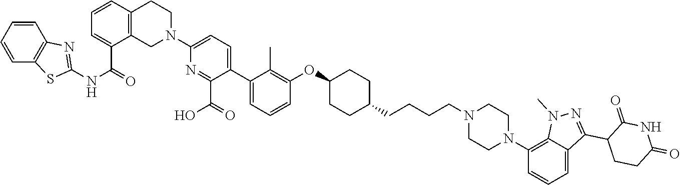 [SMILES: Cc1c(O[C@H]2CC[C@H](CCCCN3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 205 | 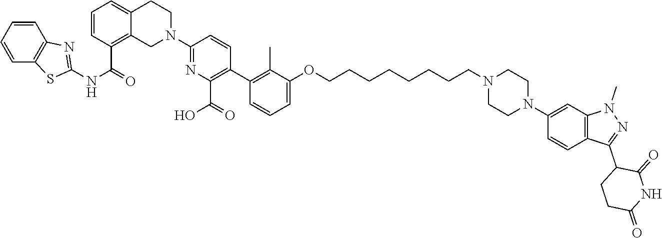 [SMILES: Cc1c(OCCCCCCCCN2CCN(c3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 206 | 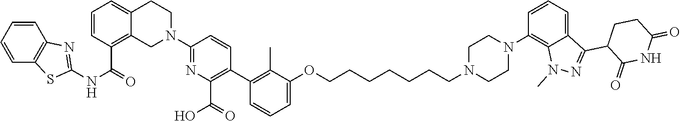 [SMILES: Cc1c(OCCCCCCCN2CCN(c3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| 207 | 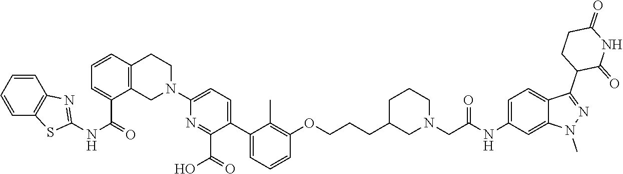 [SMILES: Cc1c(OCCCC2CCCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| 207a | 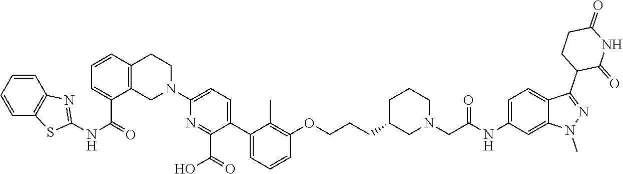 [SMILES: Cc1c(OCCC[C@@H]2CCCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| 207b | 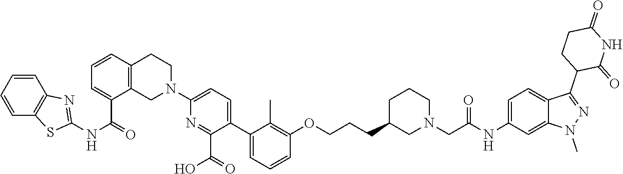 [SMILES: Cc1c(OCCC[C@H]2CCCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| 208 | 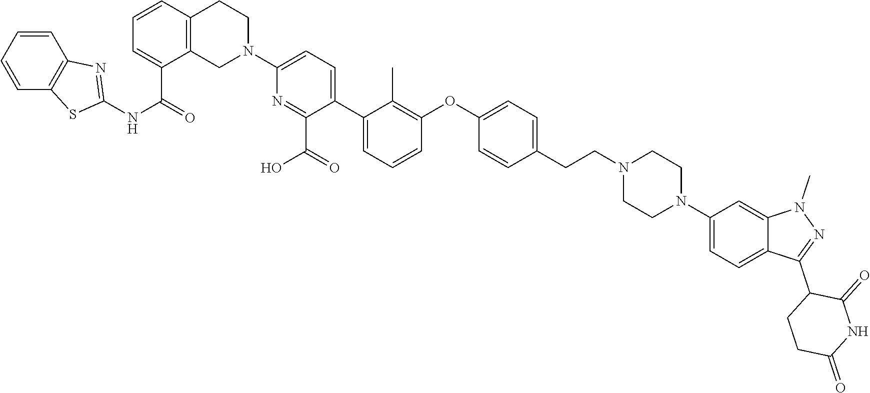 [SMILES: Cc1c(Oc2ccc(CCN3CCN(c4ccc5c(C6CCC(=O)NC6=O)nn(C)c5c4)CC3)cc2)cccc1-c1ccc(N2CCc3cccc(C(=O)Nc4nc5ccccc5s4)c3C2)nc1C(=O)O] |
| 209 | 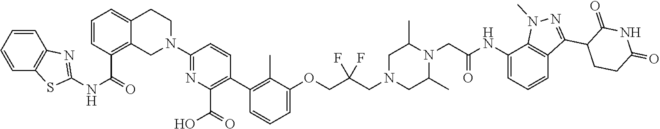 [SMILES: Cc1c(OCC(F)(F)CN2CC(C)N(CC(=O)Nc3cccc4c(C5CCC(=O)NC5=O)nn(C)c34)C(C)C2)cccc1-c1ccc(N2CCc3cccc(C(=O)Nc4nc5ccccc5s4)c3C2)nc1C(=O)O] |
| 209a | 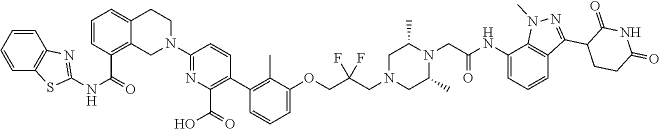 [SMILES: Cc1c(OCC(F)(F)CN2C[C@@H](C)N(CC(=O)Nc3cccc4c(C5CCC(=O)NC5=O)nn(C)c34)[C@@H](C)C2)cccc1-c1ccc(N2CCc3cccc(C(=O)Nc4nc5ccccc5s4)c3C2)nc1C(=O)O] |
| 210 | 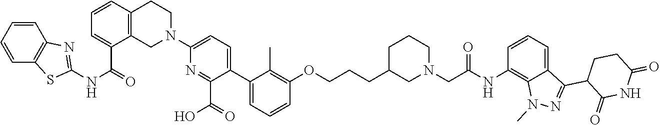 [SMILES: Cc1c(OCCCC2CCCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O] |
| 210a | 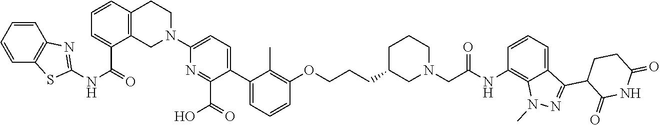 [SMILES: Cc1c(OCCC[C@@H]2CCCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O] |
| 210b | 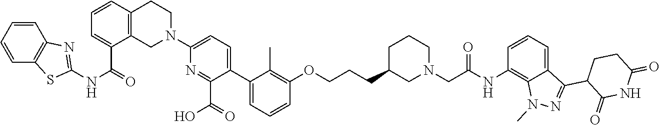 [SMILES: Cc1c(OCCC[C@H]2CCCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O] |
| 211 | 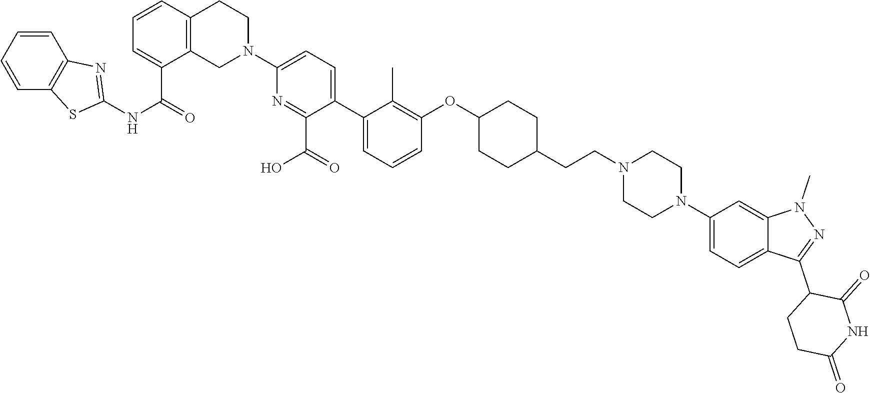 [SMILES: Cc1c(OC2CCC(CCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 211a | 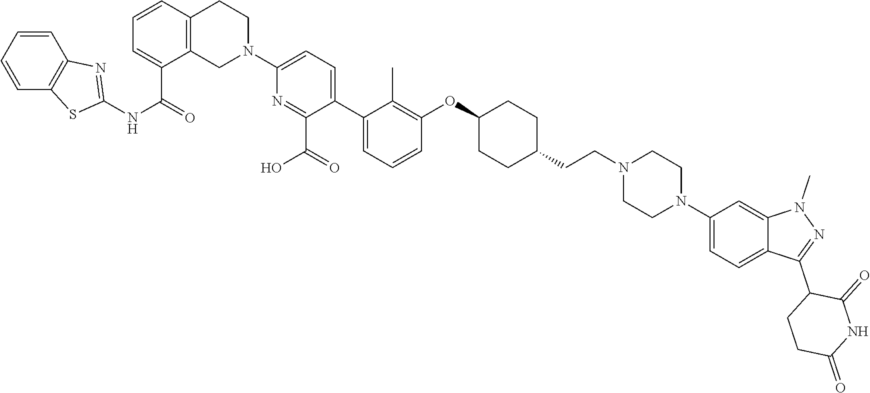 [SMILES: Cc1c(O[C@H]2CC[C@H](CCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 212 | 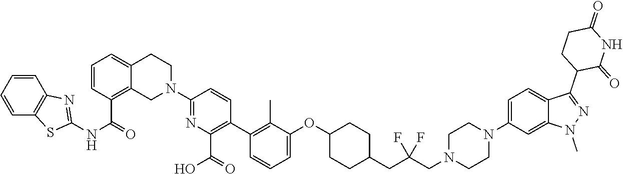 [SMILES: Cc1c(OC2CCC(CC(F)(F)CN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 212a | 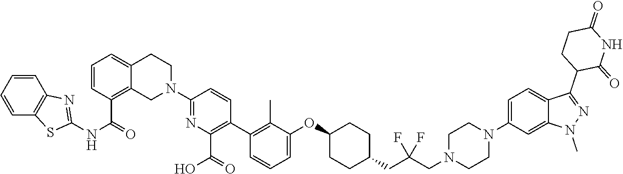 [SMILES: Cc1c(O[C@H]2CC[C@H](CC(F)(F)CN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 213 | 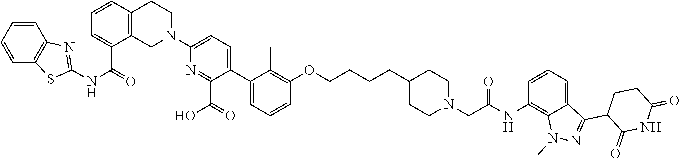 [SMILES: Cc1c(OCCCCC2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| 214 | 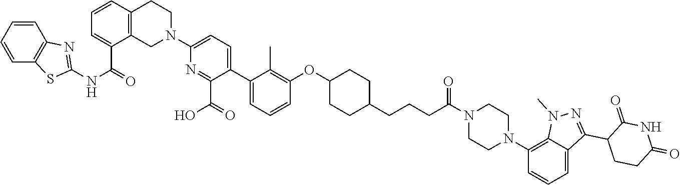 [SMILES: Cc1c(OC2CCC(CCCC(=O)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 214a | 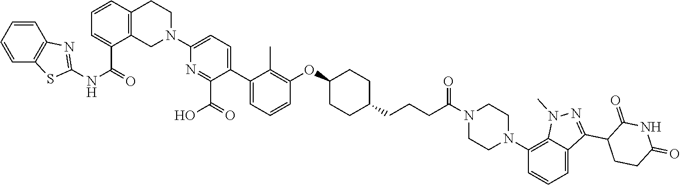 [SMILES: Cc1c(O[C@H]2CC[C@H](CCCC(=O)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 215 | 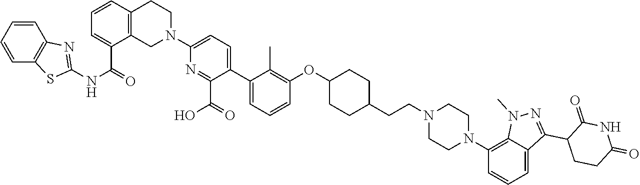 [SMILES: Cc1c(OC2CCC(CCN3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 215a | 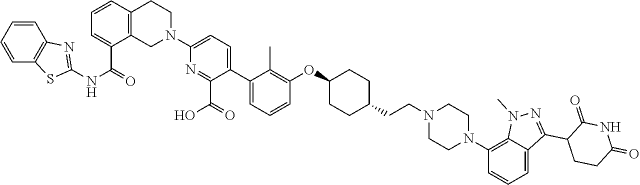 [SMILES: Cc1c(O[C@H]2CC[C@H](CCN3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 216 | 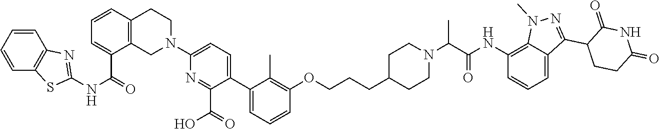 [SMILES: Cc1c(OCCCC2CCN(C(C)C(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| 216a | 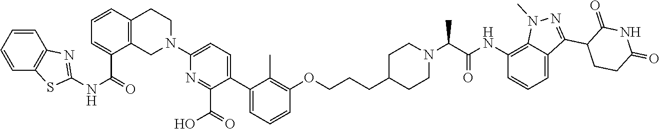 [SMILES: Cc1c(OCCCC2CCN([C@@H](C)C(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| 216b | 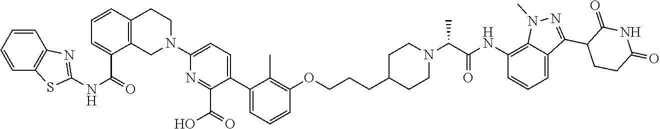 [SMILES: Cc1c(OCCCC2CCN([C@H](C)C(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| 217 | 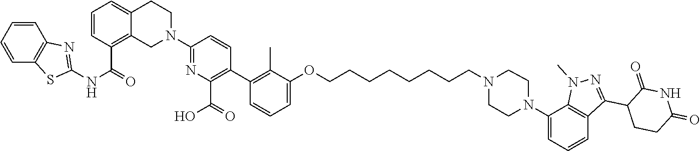 [SMILES: Cc1c(OCCCCCCCCN2CCN(c3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| 218 | 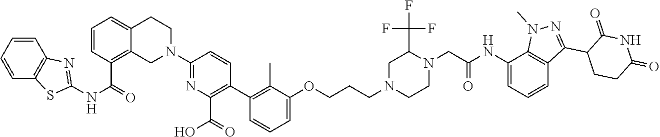 [SMILES: Cc1c(OCCCN2CCN(CC(=O)Nc3cccc4c(C5CCC(=O)NC5=O)nn(C)c34)C(C(F)(F)F)C2)cccc1-c1ccc(N2CCc3cccc(C(=O)Nc4nc5ccccc5s4)c3C2)nc1C(=O)O] |
| 218a | 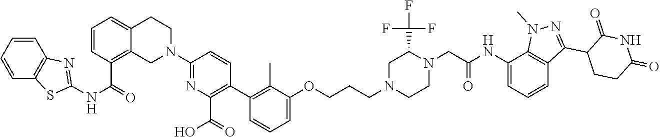 [SMILES: Cc1c(OCCCN2CCN(CC(=O)Nc3cccc4c(C5CCC(=O)NC5=O)nn(C)c34)[C@@H](C(F)(F)F)C2)cccc1-c1ccc(N2CCc3cccc(C(=O)Nc4nc5ccccc5s4)c3C2)nc1C(=O)O] |
| 218b | 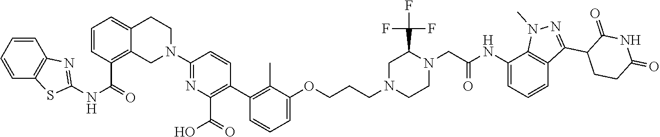 [SMILES: Cc1c(OCCCN2CCN(CC(=O)Nc3cccc4c(C5CCC(=O)NC5=O)nn(C)c34)[C@H](C(F)(F)F)C2)cccc1-c1ccc(N2CCc3cccc(C(=O)Nc4nc5ccccc5s4)c3C2)nc1C(=O)O] |
| 219 | 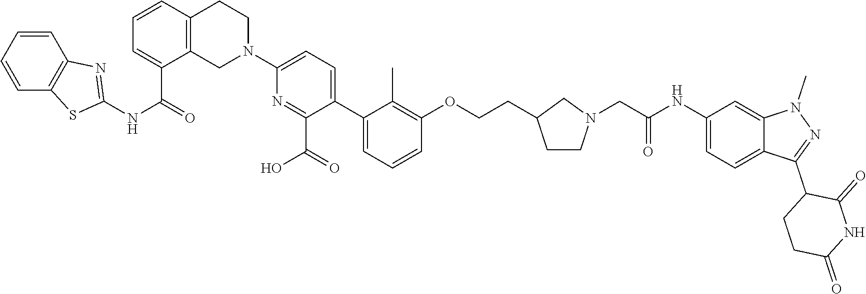 [SMILES: Cc1c(OCCC2CCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| 219a | 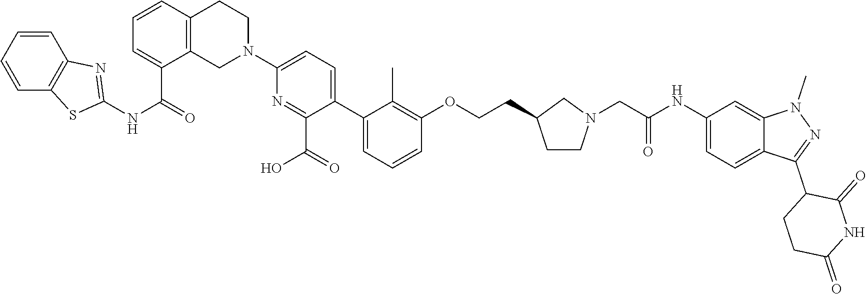 [SMILES: Cc1c(OCC[C@@H]2CCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| 219b | 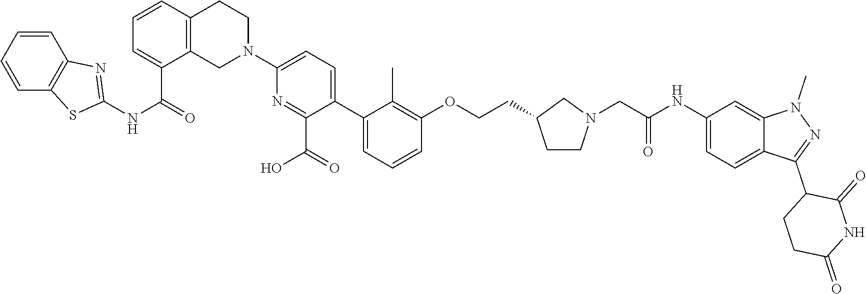 [SMILES: Cc1c(OCC[C@H]2CCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] |
| 220 | 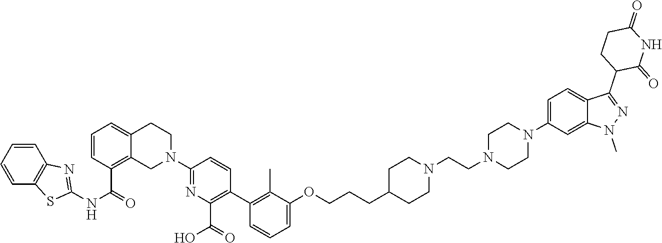 [SMILES: Cc1c(OCCCC2CCN(CCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 221 | 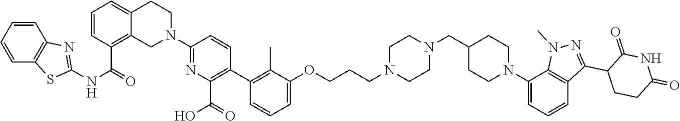 [SMILES: Cc1c(OCCCN2CCN(CC3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 222 | 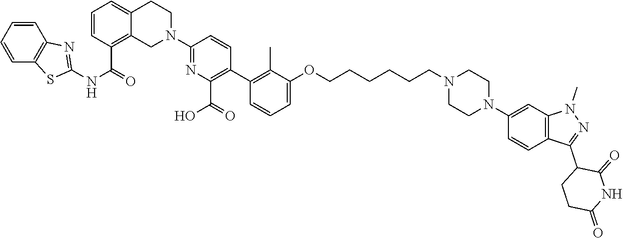 [SMILES: Cc1c(OCCCCCCN2CCN(c3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 223 | 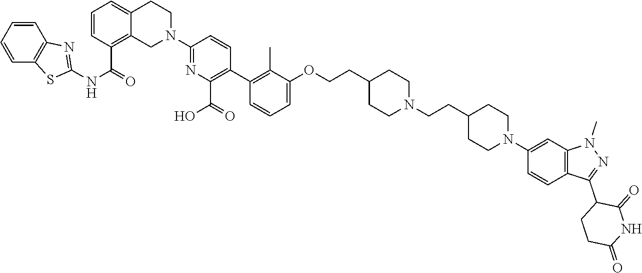 [SMILES: Cc1c(OCCC2CCN(CCC3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 224 | 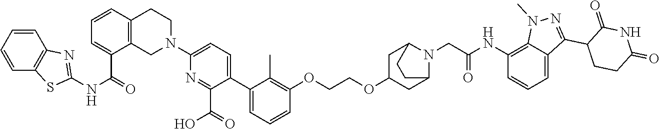 [SMILES: Cc1c(OCCOC2CC3CCC(C2)N3CC(=O)Nc2cccc3c(C4CCC(=O)NC4=O)nn(C)c23)cccc1-c1ccc(N2CCc3cccc(C(=O)Nc4nc5ccccc5s4)c3C2)nc1C(=O)O] |
| 224a | 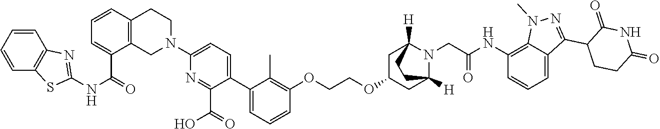 [SMILES: Cc1c(OCCO[C@@H]2C[C@H]3CC[C@@H](C2)N3CC(=O)Nc2cccc3c(C4CCC(=O)NC4=O)nn(C)c23)cccc1-c1ccc(N2CCc3cccc(C(=O)Nc4nc5ccccc5s4)c3C2)nc1C(=O)O] |
| 224b | 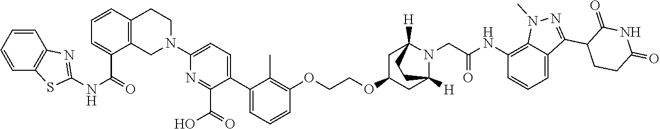 [SMILES: Cc1c(OCCO[C@H]2C[C@H]3CC[C@@H](C2)N3CC(=O)Nc2cccc3c(C4CCC(=O)NC4=O)nn(C)c23)cccc1-c1ccc(N2CCc3cccc(C(=O)Nc4nc5ccccc5s4)c3C2)nc1C(=O)O] |
| 225 | 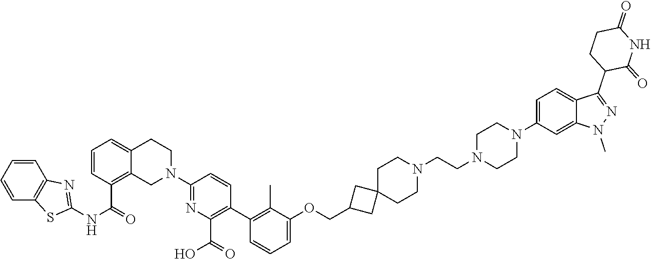 [SMILES: Cc1c(OCC2CC3(CCN(CCN4CCN(c5ccc6c(C7CCC(=O)NC7=O)nn(C)c6c5)CC4)CC3)C2)cccc1-c1ccc(N2CCc3cccc(C(=O)Nc4nc5ccccc5s4)c3C2)nc1C(=O)O] |
| 226 | 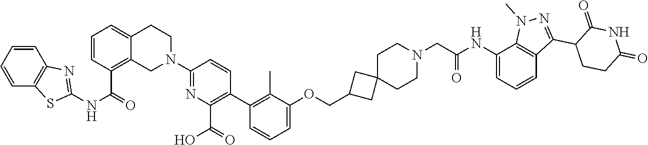 [SMILES: Cc1c(OCC2CC3(CCN(CC(=O)Nc4cccc5c(C6CCC(=O)NC6=O)nn(C)c45)CC3)C2)cccc1-c1ccc(N2CCc3cccc(C(=O)Nc4nc5ccccc5s4)c3C2)nc1C(=O)O] |
| 227 | 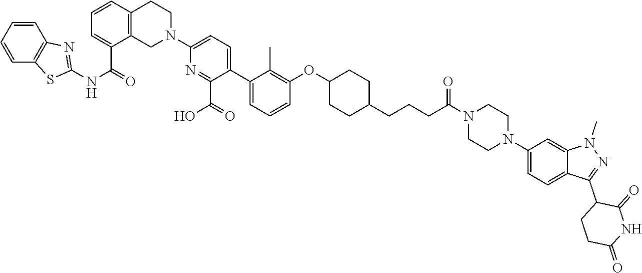 [SMILES: Cc1c(OC2CCC(CCCC(=O)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 227a | 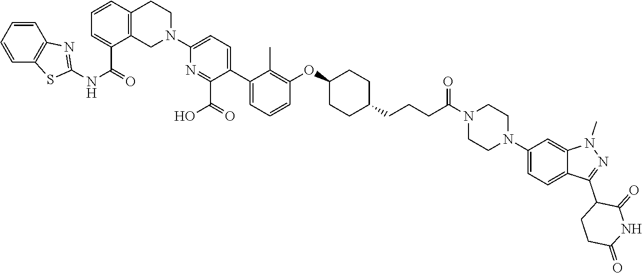 [SMILES: Cc1c(O[C@H]2CC[C@H](CCCC(=O)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 228 | 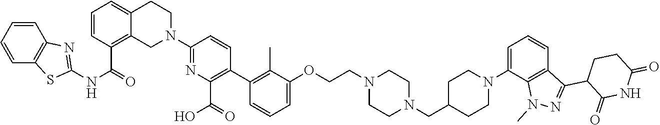 [SMILES: Cc1c(OCCN2CCN(CC3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 229 | 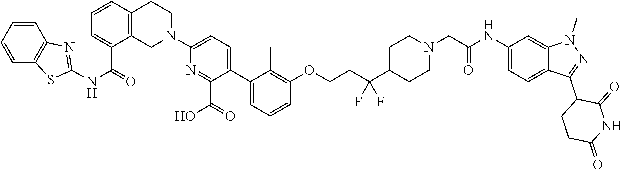 [SMILES: Cc1c(OCCC(F)(F)C2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 230 | 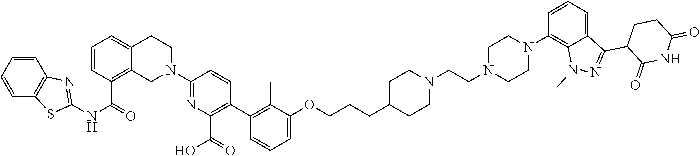 [SMILES: Cc1c(OCCCC2CCN(CCN3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 231 | 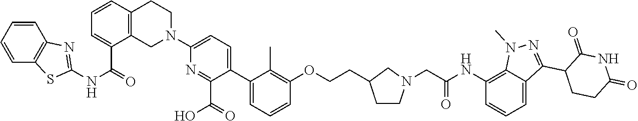 [SMILES: Cc1c(OCCC2CCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O] |
| 231a | 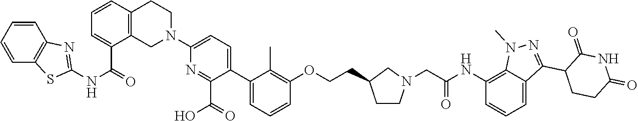 [SMILES: Cc1c(OCC[C@@H]2CCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O] |
| 231b | 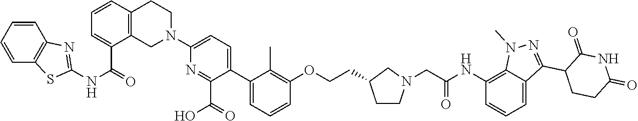 [SMILES: Cc1c(OCC[C@H]2CCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O] |
| 232 | 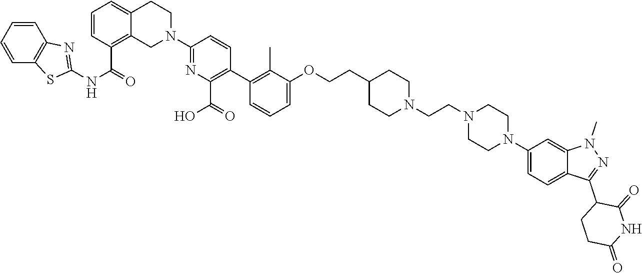 [SMILES: Cc1c(OCCC2CCN(CCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 233 | 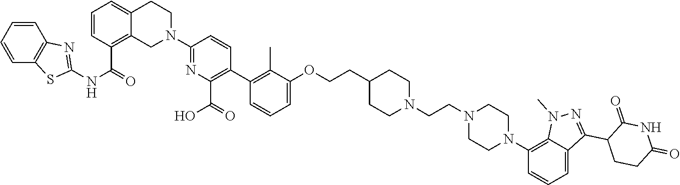 [SMILES: Cc1c(OCCC2CCN(CCN3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 234 | 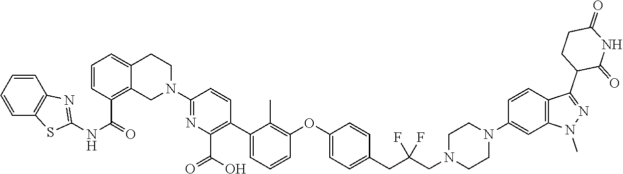 [SMILES: Cc1c(Oc2ccc(CC(F)(F)CN3CCN(c4ccc5c(C6CCC(=O)NC6=O)nn(C)c5c4)CC3)cc2)cccc1-c1ccc(N2CCc3cccc(C(=O)Nc4nc5ccccc5s4)c3C2)nc1C(=O)O] |
| 235 | 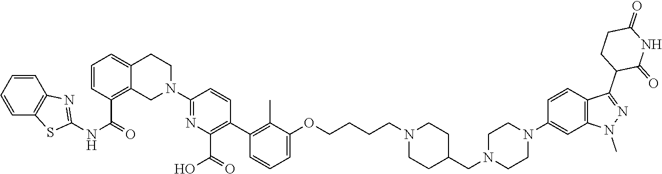 [SMILES: Cc1c(OCCCCN2CCC(CN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 236 | 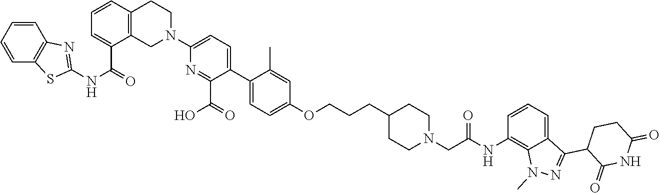 [SMILES: Cc1cc(OCCCC2CCN(CC(=O)Nc3cccc4c(C5CCC(=O)NC5=O)nn(C)c34)CC2)ccc1-c1ccc(N2CCc3cccc(C(=O)Nc4nc5ccccc5s4)c3C2)nc1C(=O)O] |
| 237 | 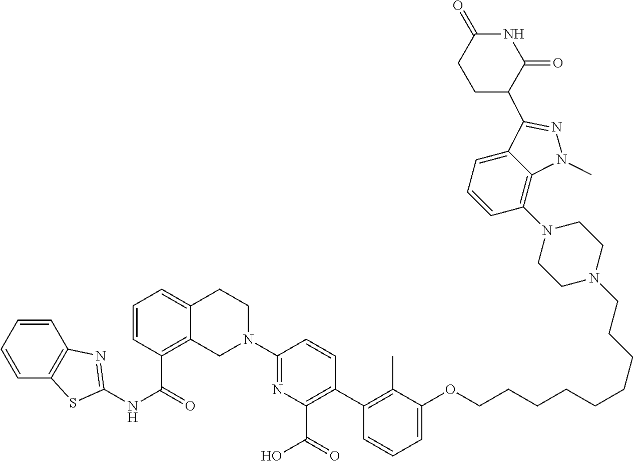 [SMILES: Cc1c(OCCCCCCCCCN2CCN(c3cccc4c(C5CCC(=O)NC5=O)nn(C)c34)CC2)cccc1-c1ccc(N2CCc3cccc(C(=O)Nc4nc5ccccc5s4)c3C2)nc1C(=O)O] |
| 238 | 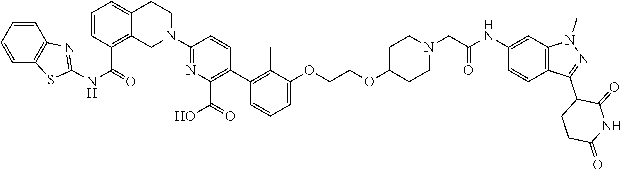 [SMILES: Cc1c(OCCOC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 239 | 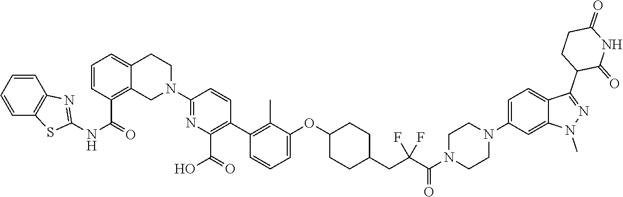 [SMILES: Cc1c(OC2CCC(CC(F)(F)C(=O)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 239a | 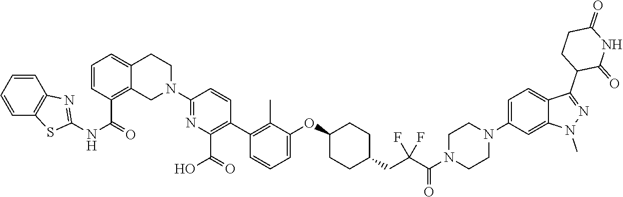 [SMILES: Cc1c(O[C@H]2CC[C@H](CC(F)(F)C(=O)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 240 | 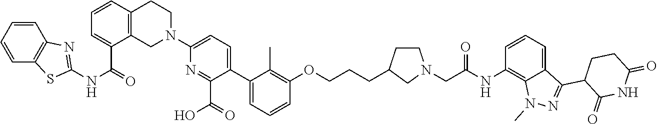 [SMILES: Cc1c(OCCCC2CCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O] |
| 240a | 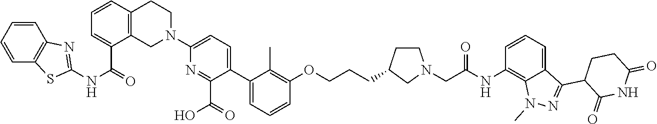 [SMILES: Cc1c(OCCC[C@@H]2CCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O] |
| 240b | 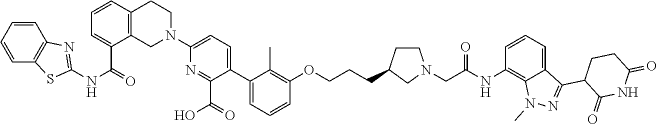 [SMILES: Cc1c(OCCC[C@H]2CCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O] |
| 241 | 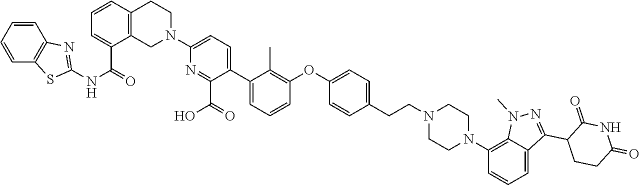 [SMILES: Cc1c(Oc2ccc(CCN3CCN(c4cccc5c(C6CCC(=O)NC6=O)nn(C)c45)CC3)cc2)cccc1-c1ccc(N2CCc3cccc(C(=O)Nc4nc5ccccc5s4)c3C2)nc1C(=O)O] |
| 242 | 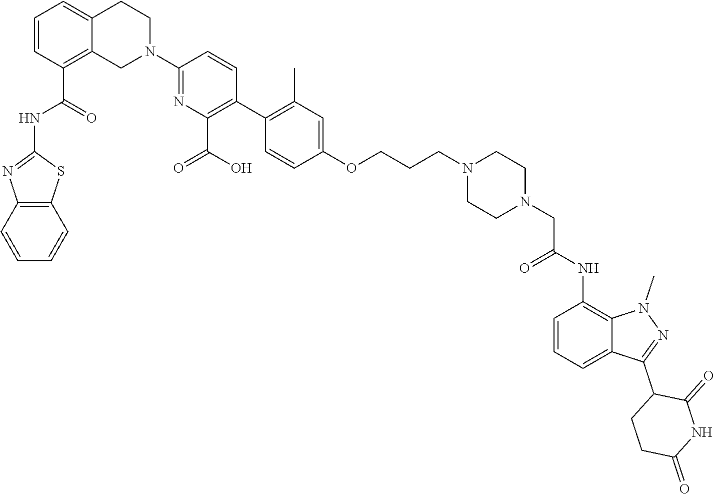 [SMILES: Cc1cc(OCCCN2CCN(CC(=O)Nc3cccc4c(C5CCC(=O)NC5=O)nn(C)c34)CC2)ccc1-c1ccc(N2CCc3cccc(C(=O)Nc4nc5ccccc5s4)c3C2)nc1C(=O)O] |
| 243 | 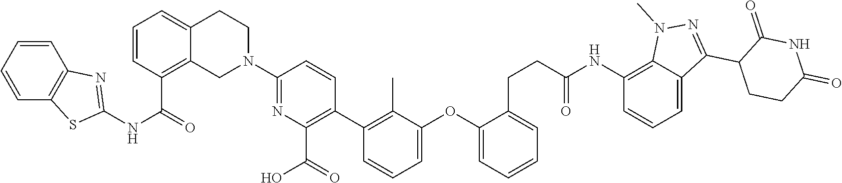 [SMILES: Cc1c(Oc2ccccc2CCC(=O)Nc2cccc3c(C4CCC(=O)NC4=O)nn(C)c23)cccc1-c1ccc(N2CCc3cccc(C(=O)Nc4nc5ccccc5s4)c3C2)nc1C(=O)O] |
| 244 | 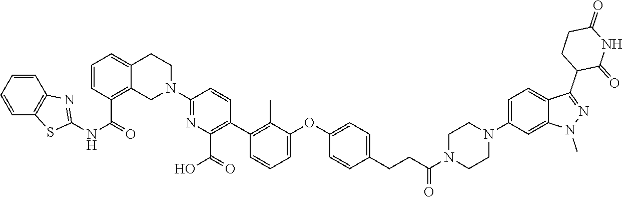 [SMILES: Cc1c(Oc2ccc(CCC(=O)N3CCN(c4ccc5c(C6CCC(=O)NC6=O)nn(C)c5c4)CC3)cc2)cccc1-c1ccc(N2CCc3cccc(C(=O)Nc4nc5ccccc5s4)c3C2)nc1C(=O)O] |
| 245 | 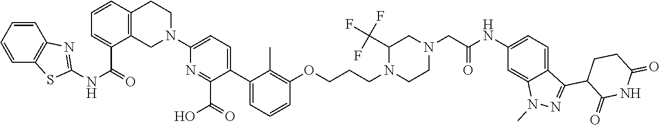 [SMILES: Cc1c(OCCCN2CCN(CC(=O)Nc3ccc4c(C5CCC(=O)NC5=O)nn(C)c4c3)CC2C(F)(F)F)cccc1-c1ccc(N2CCc3cccc(C(=O)Nc4nc5ccccc5s4)c3C2)nc1C(=O)O] |
| 245a | 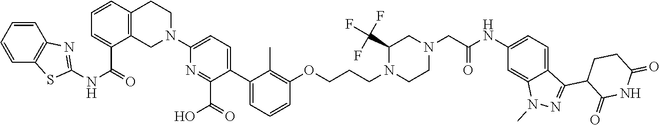 [SMILES: Cc1c(OCCCN2CCN(CC(=O)Nc3ccc4c(C5CCC(=O)NC5=O)nn(C)c4c3)C[C@@H]2C(F)(F)F)cccc1-c1ccc(N2CCc3cccc(C(=O)Nc4nc5ccccc5s4)c3C2)nc1C(=O)O] |
| 245b | 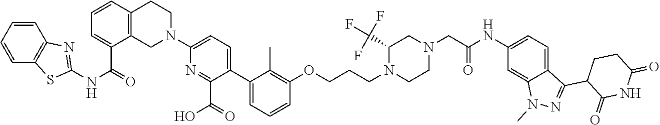 [SMILES: Cc1c(OCCCN2CCN(CC(=O)Nc3ccc4c(C5CCC(=O)NC5=O)nn(C)c4c3)C[C@H]2C(F)(F)F)cccc1-c1ccc(N2CCc3cccc(C(=O)Nc4nc5ccccc5s4)c3C2)nc1C(=O)O] |
| 246 | 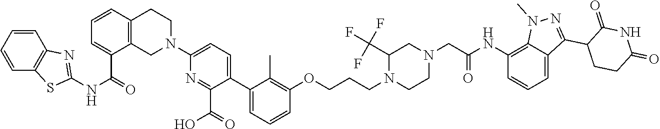 [SMILES: Cc1c(OCCCN2CCN(CC(=O)Nc3cccc4c(C5CCC(=O)NC5=O)nn(C)c34)CC2C(F)(F)F)cccc1-c1ccc(N2CCc3cccc(C(=O)Nc4nc5ccccc5s4)c3C2)nc1C(=O)O] |
| 246a | 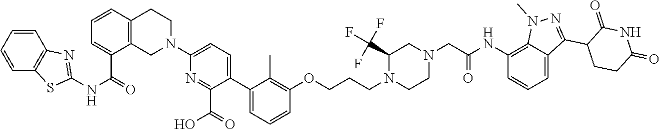 [SMILES: Cc1c(OCCCN2CCN(CC(=O)Nc3cccc4c(C5CCC(=O)NC5=O)nn(C)c34)C[C@@H]2C(F)(F)F)cccc1-c1ccc(N2CCc3cccc(C(=O)Nc4nc5ccccc5s4)c3C2)nc1C(=O)O] |
| 246b | 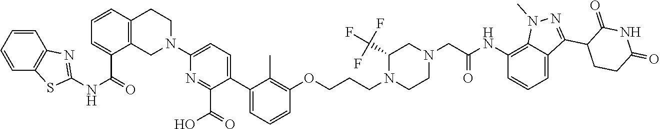 [SMILES: Cc1c(OCCCN2CCN(CC(=O)Nc3cccc4c(C5CCC(=O)NC5=O)nn(C)c34)C[C@H]2C(F)(F)F)cccc1-c1ccc(N2CCc3cccc(C(=O)Nc4nc5ccccc5s4)c3C2)nc1C(=O)O] |
| 247 | 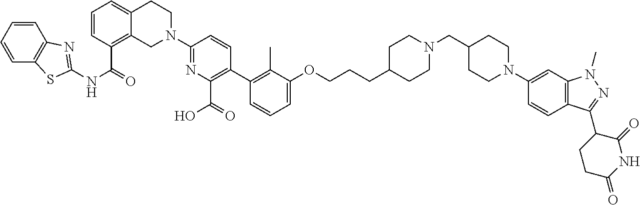 [SMILES: Cc1c(OCCCC2CCN(CC3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 248 | 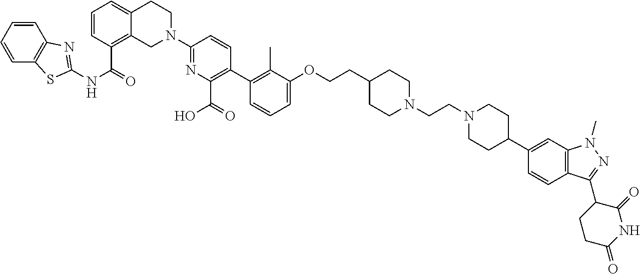 [SMILES: Cc1c(OCCC2CCN(CCN3CCC(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 249 | 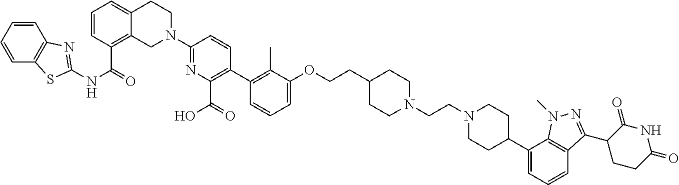 [SMILES: Cc1c(OCCC2CCN(CCN3CCC(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 250 | 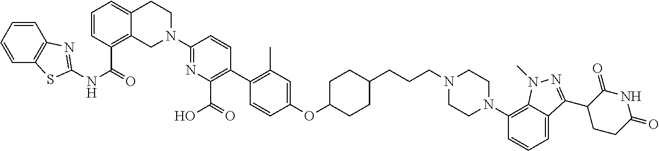 [SMILES: Cc1cc(OC2CCC(CCCN3CCN(c4cccc5c(C6CCC(=O)NC6=O)nn(C)c45)CC3)CC2)ccc1-c1ccc(N2CCc3cccc(C(=O)Nc4nc5ccccc5s4)c3C2)nc1C(=O)O] |
| 250a | 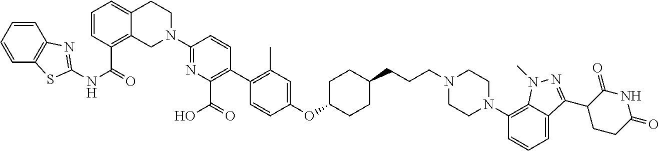 [SMILES: Cc1cc(O[C@H]2CC[C@H](CCCN3CCN(c4cccc5c(C6CCC(=O)NC6=O)nn(C)c45)CC3)CC2)ccc1-c1ccc(N2CCc3cccc(C(=O)Nc4nc5ccccc5s4)c3C2)nc1C(=O)O] |
| 251 | 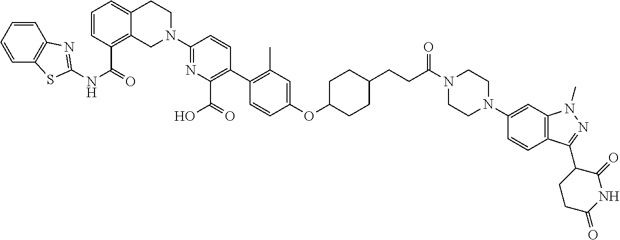 [SMILES: Cc1cc(OC2CCC(CCC(=O)N3CCN(c4ccc5c(C6CCC(=O)NC6=O)nn(C)c5c4)CC3)CC2)ccc1-c1ccc(N2CCc3cccc(C(=O)Nc4nc5ccccc5s4)c3C2)nc1C(=O)O] |
| 251a | 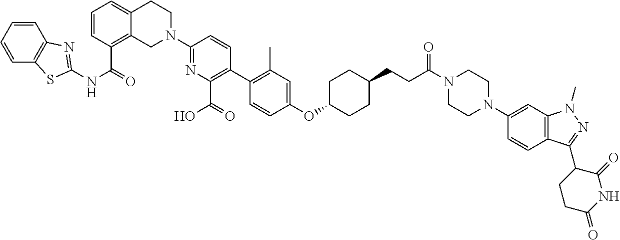 [SMILES: Cc1cc(O[C@H]2CC[C@H](CCC(=O)N3CCN(c4ccc5c(C6CCC(=O)NC6=O)nn(C)c5c4)CC3)CC2)ccc1-c1ccc(N2CCc3cccc(C(=O)Nc4nc5ccccc5s4)c3C2)nc1C(=O)O] |
| 252 | 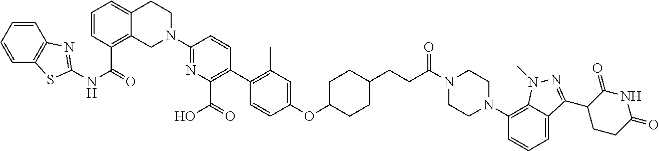 [SMILES: Cc1cc(OC2CCC(CCC(=O)N3CCN(c4cccc5c(C6CCC(=O)NC6=O)nn(C)c45)CC3)CC2)ccc1-c1ccc(N2CCc3cccc(C(=O)Nc4nc5ccccc5s4)c3C2)nc1C(=O)O] |
| 252a | 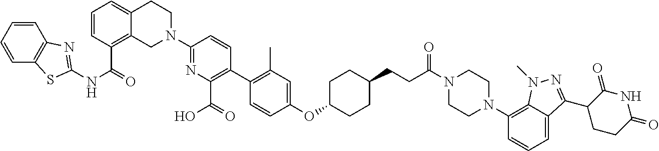 [SMILES: Cc1cc(O[C@H]2CC[C@H](CCC(=O)N3CCN(c4cccc5c(C6CCC(=O)NC6=O)nn(C)c45)CC3)CC2)ccc1-c1ccc(N2CCc3cccc(C(=O)Nc4nc5ccccc5s4)c3C2)nc1C(=O)O] |
| 253 | 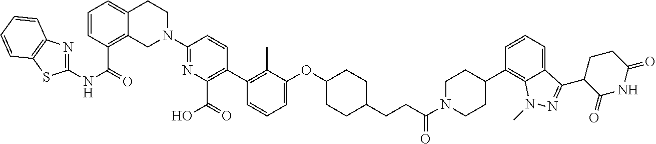 [SMILES: Cc1c(OC2CCC(CCC(=O)N3CCC(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 253a | 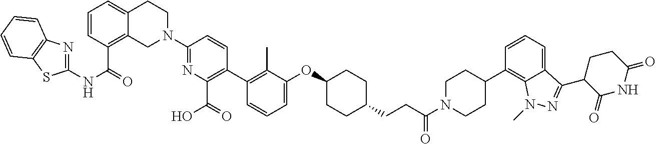 [SMILES: Cc1c(O[C@H]2CC[C@H](CCC(=O)N3CCC(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 254 | 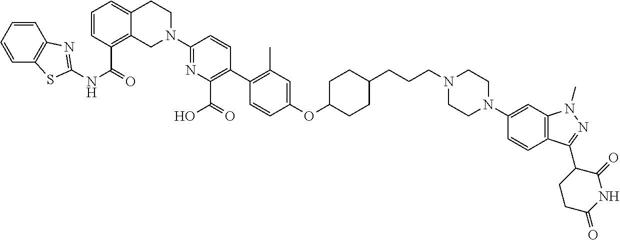 [SMILES: Cc1cc(OC2CCC(CCCN3CCN(c4ccc5c(C6CCC(=O)NC6=O)nn(C)c5c4)CC3)CC2)ccc1-c1ccc(N2CCc3cccc(C(=O)Nc4nc5ccccc5s4)c3C2)nc1C(=O)O] |
| 254a | 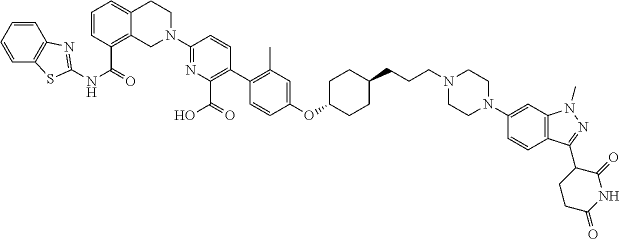 [SMILES: Cc1cc(O[C@H]2CC[C@H](CCCN3CCN(c4ccc5c(C6CCC(=O)NC6=O)nn(C)c5c4)CC3)CC2)ccc1-c1ccc(N2CCc3cccc(C(=O)Nc4nc5ccccc5s4)c3C2)nc1C(=O)O] |
| 255 | 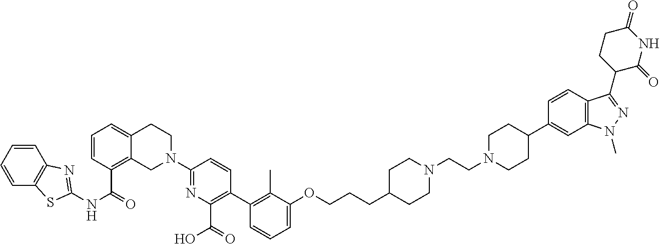 [SMILES: Cc1c(OCCCC2CCN(CCN3CCC(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 256 | 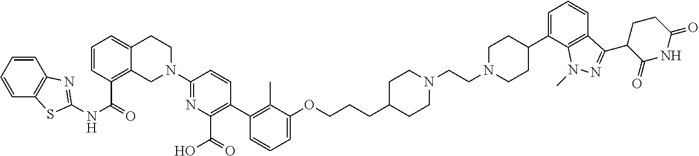 [SMILES: Cc1c(OCCCC2CCN(CCN3CCC(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 257 | 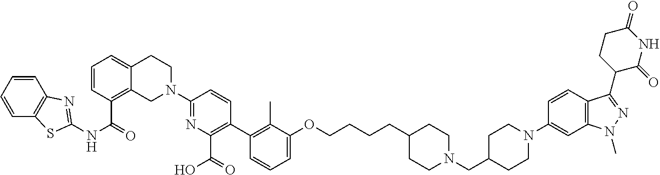 [SMILES: Cc1c(OCCCCC2CCN(CC3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 258 | 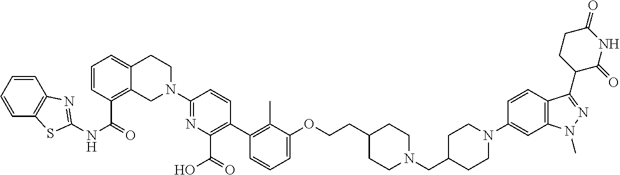 [SMILES: Cc1c(OCCC2CCN(CC3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 259 | 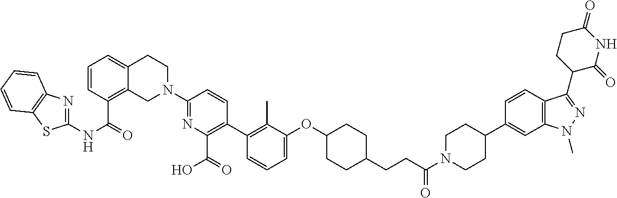 [SMILES: Cc1c(OC2CCC(CCC(=O)N3CCC(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 259a | 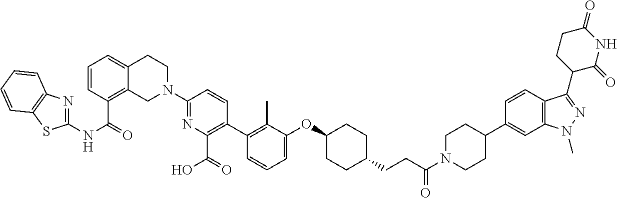 [SMILES: Cc1c(O[C@H]2CC[C@H](CCC(=O)N3CCC(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 260 | 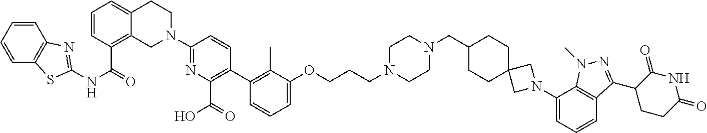 [SMILES: Cc1c(OCCCN2CCN(CC3CCC4(CC3)CN(c3cccc5c(C6CCC(=O)NC6=O)nn(C)c35)C4)CC2)cccc1-c1ccc(N2CCc3cccc(C(=O)Nc4nc5ccccc5s4)c3C2)nc1C(=O)O] |
| 261 | 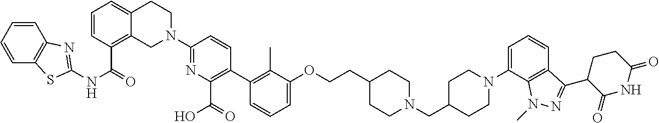 [SMILES: Cc1c(OCCC2CCN(CC3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 262 | 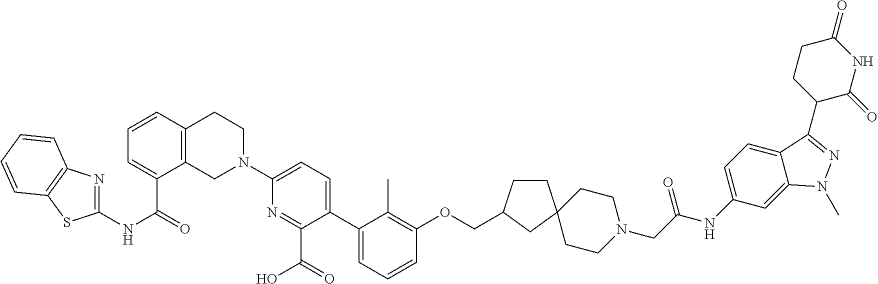 [SMILES: Cc1c(OCC2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| 262a | 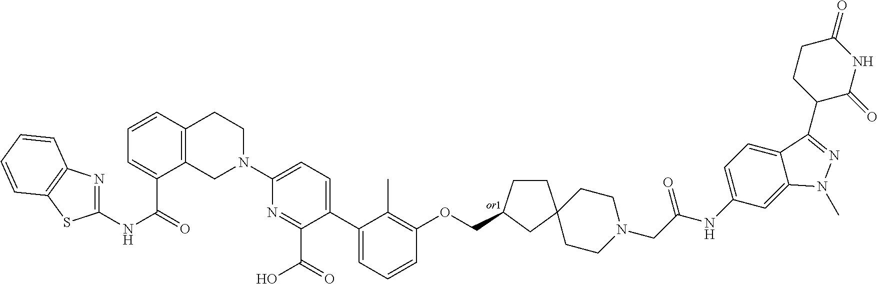 [SMILES: Cc1c(OC[C@H]2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| 262b | 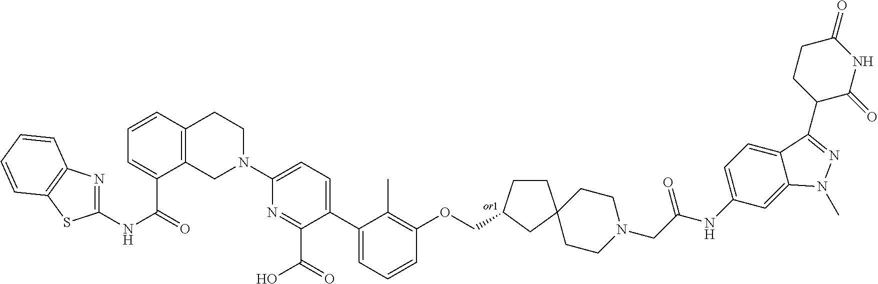 [SMILES: Cc1c(OC[C@@H]2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| 263 | 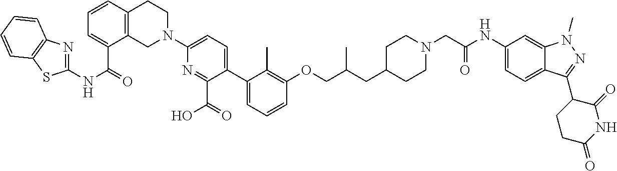 [SMILES: Cc1c(OCC(C)CC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 263a | 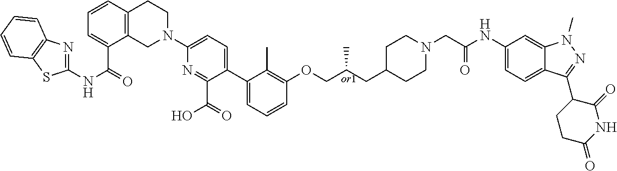 [SMILES: Cc1c(OC[C@H](C)CC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 263b | 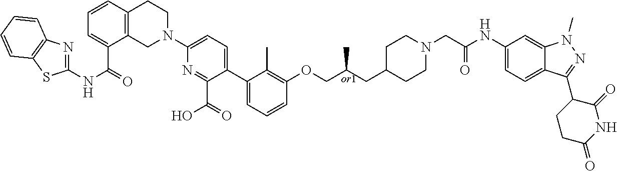 [SMILES: Cc1c(OC[C@@H](C)CC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 264 | 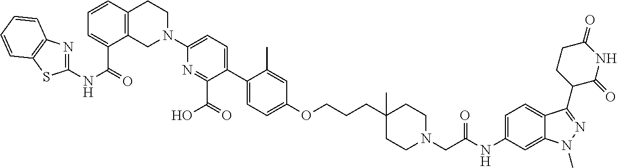 [SMILES: Cc1cc(OCCCC2(C)CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 265 | 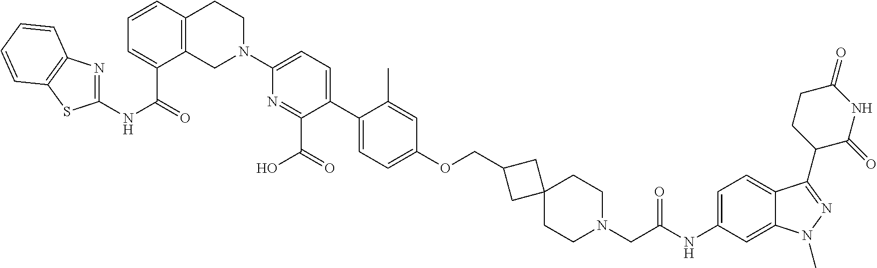 [SMILES: Cc1cc(OCC2CC3(CCN(CC(=O)Nc4ccc5c(C6CCC(=O)NC6=O)nn(C)c5c4)CC3)C2)ccc1-c1ccc(N2CCc3cccc(C(=O)Nc4nc5ccccc5s4)c3C2)nc1C(=O)O] |
| 266 | 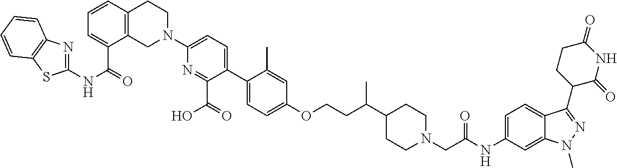 [SMILES: Cc1cc(OCCC(C)C2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 266a | 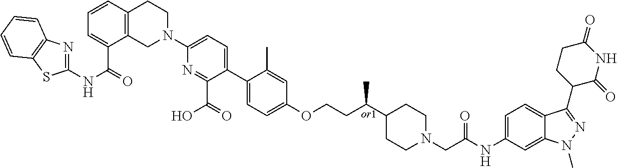 [SMILES: Cc1cc(OCC[C@@H](C)C2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 266b | 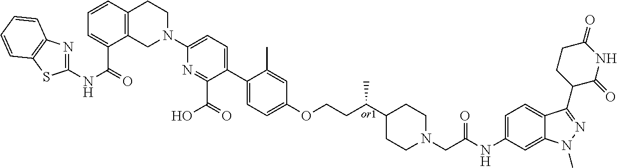 [SMILES: Cc1cc(OCC[C@H](C)C2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 267 | 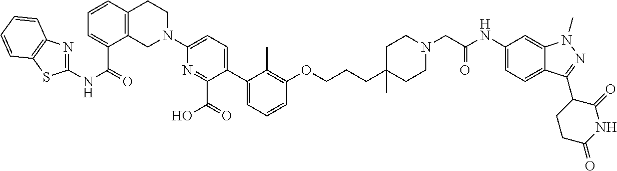 [SMILES: Cc1c(OCCCC2(C)CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 268 | 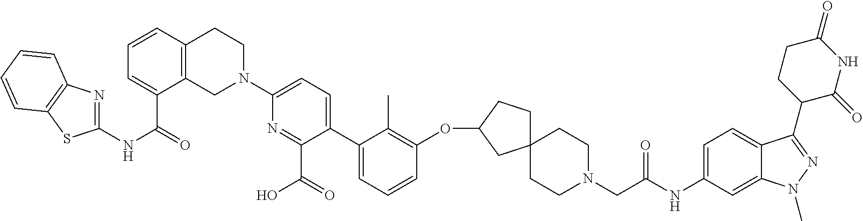 [SMILES: Cc1c(OC2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| 268a | 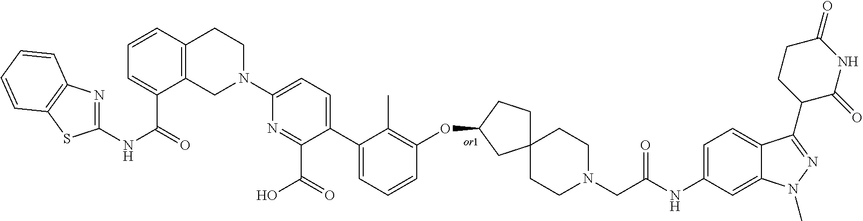 [SMILES: Cc1c(O[C@H]2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| 268b | 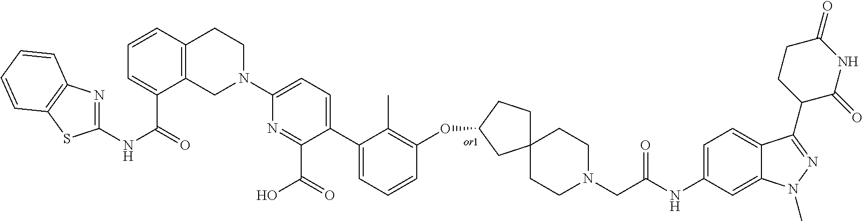 [SMILES: Cc1c(O[C@@H]2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| 269 | 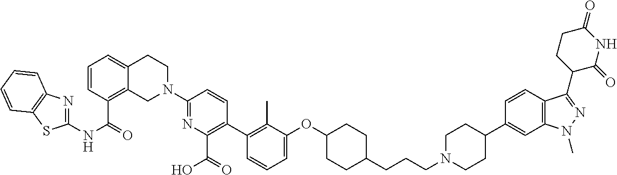 [SMILES: Cc1c(OC2CCC(CCCN3CCC(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 269a | 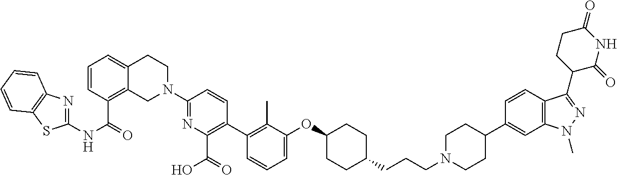 [SMILES: Cc1c(O[C@H]2CC[C@H](CCCN3CCC(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 270 | 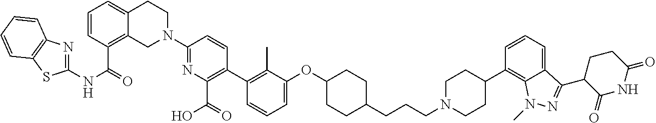 [SMILES: Cc1c(OC2CCC(CCCN3CCC(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 270a | 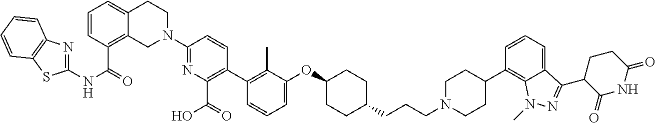 [SMILES: Cc1c(O[C@H]2CC[C@H](CCCN3CCC(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 271 | 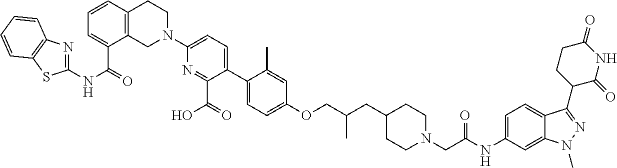 [SMILES: Cc1cc(OCC(C)CC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 271a | 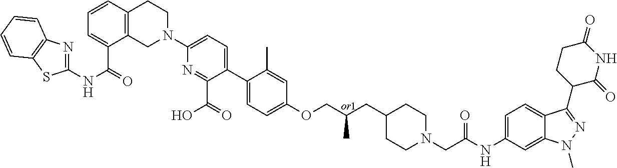 [SMILES: Cc1cc(OC[C@H](C)CC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 271b | 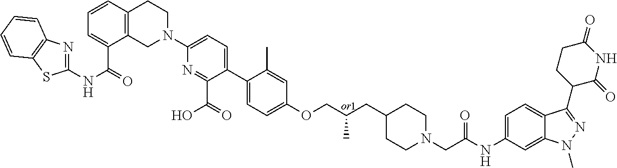 [SMILES: Cc1cc(OC[C@@H](C)CC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 272 | 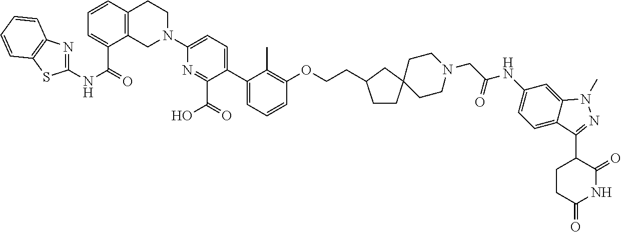 [SMILES: Cc1c(OCCC2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| 272a | 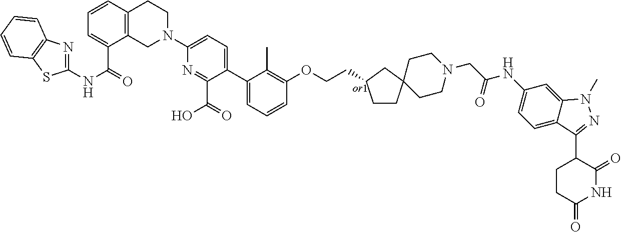 [SMILES: Cc1c(OCC[C@H]2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| 272b | 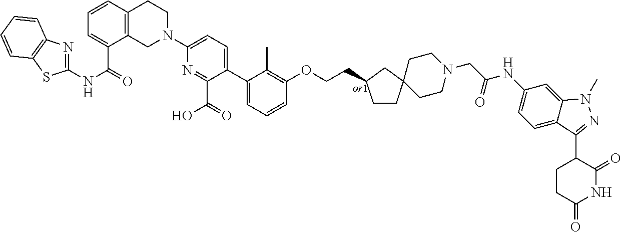 [SMILES: Cc1c(OCC[C@@H]2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| 273 | 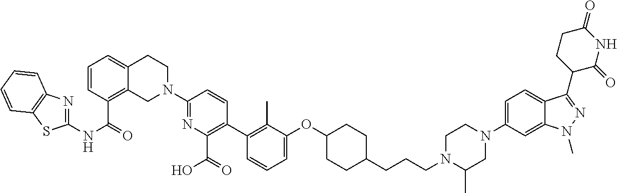 [SMILES: Cc1c(OC2CCC(CCCN3CCN(c4ccc5c(C6CCC(=O)NC6=O)nn(C)c5c4)CC3C)CC2)cccc1-c1ccc(N2CCc3cccc(C(=O)Nc4nc5ccccc5s4)c3C2)nc1C(=O)O] |
| 273a | 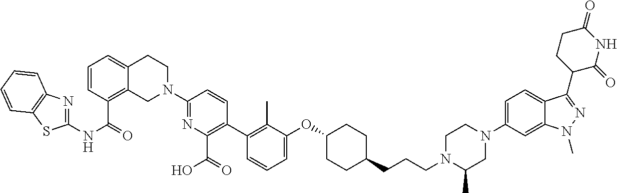 [SMILES: Cc1c(O[C@H]2CC[C@H](CCCN3CCN(c4ccc5c(C6CCC(=O)NC6=O)nn(C)c5c4)C[C@H]3C)CC2)cccc1-c1ccc(N2CCc3cccc(C(=O)Nc4nc5ccccc5s4)c3C2)nc1C(=O)O] |
| 273b | 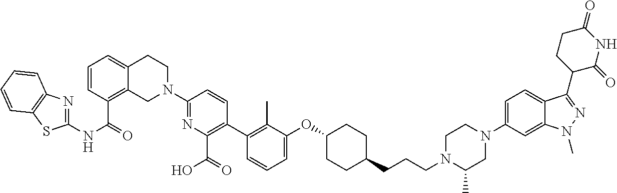 [SMILES: Cc1c(O[C@H]2CC[C@H](CCCN3CCN(c4ccc5c(C6CCC(=O)NC6=O)nn(C)c5c4)C[C@@H]3C)CC2)cccc1-c1ccc(N2CCc3cccc(C(=O)Nc4nc5ccccc5s4)c3C2)nc1C(=O)O] |
| 274 | 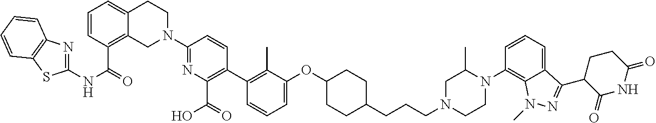 [SMILES: Cc1c(OC2CCC(CCCN3CCN(c4cccc5c(C6CCC(=O)NC6=O)nn(C)c45)C(C)C3)CC2)cccc1-c1ccc(N2CCc3cccc(C(=O)Nc4nc5ccccc5s4)c3C2)nc1C(=O)O] |
| 274a | 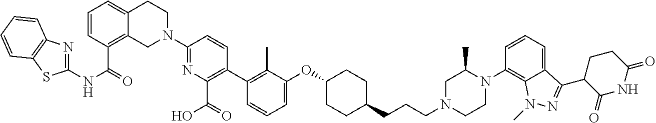 [SMILES: Cc1c(O[C@H]2CC[C@H](CCCN3CCN(c4cccc5c(C6CCC(=O)NC6=O)nn(C)c45)[C@H](C)C3)CC2)cccc1-c1ccc(N2CCc3cccc(C(=O)Nc4nc5ccccc5s4)c3C2)nc1C(=O)O] |
| 274b | 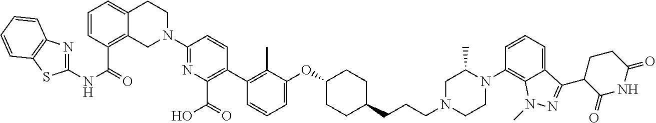 [SMILES: Cc1c(O[C@H]2CC[C@H](CCCN3CCN(c4cccc5c(C6CCC(=O)NC6=O)nn(C)c45)[C@@H](C)C3)CC2)cccc1-c1ccc(N2CCc3cccc(C(=O)Nc4nc5ccccc5s4)c3C2)nc1C(=O)O] |
| 275 | 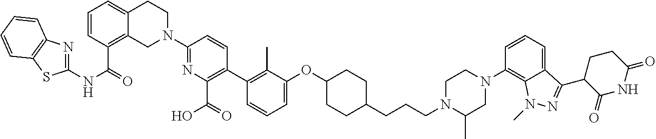 [SMILES: Cc1c(OC2CCC(CCCN3CCN(c4cccc5c(C6CCC(=O)NC6=O)nn(C)c45)CC3C)CC2)cccc1-c1ccc(N2CCc3cccc(C(=O)Nc4nc5ccccc5s4)c3C2)nc1C(=O)O] |
| 275a | 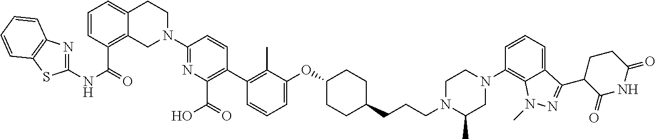 [SMILES: Cc1c(O[C@H]2CC[C@H](CCCN3CCN(c4cccc5c(C6CCC(=O)NC6=O)nn(C)c45)C[C@H]3C)CC2)cccc1-c1ccc(N2CCc3cccc(C(=O)Nc4nc5ccccc5s4)c3C2)nc1C(=O)O] |
| 275b | 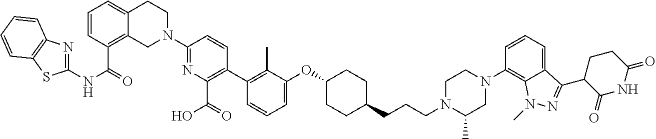 [SMILES: Cc1c(O[C@H]2CC[C@H](CCCN3CCN(c4cccc5c(C6CCC(=O)NC6=O)nn(C)c45)C[C@@H]3C)CC2)cccc1-c1ccc(N2CCc3cccc(C(=O)Nc4nc5ccccc5s4)c3C2)nc1C(=O)O] |
| 276 | 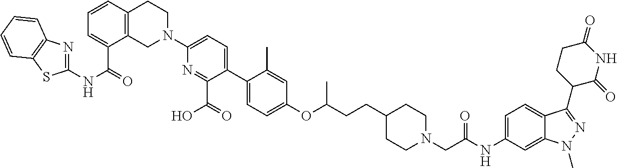 [SMILES: Cc1cc(OC(C)CCC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 276a | 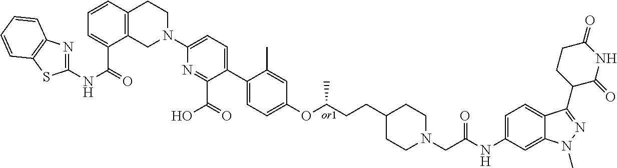 [SMILES: Cc1cc(O[C@H](C)CCC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 276b | 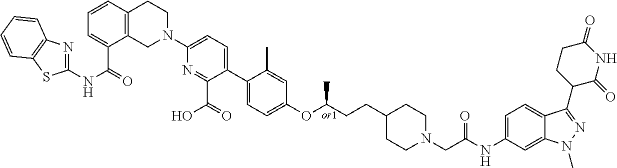 [SMILES: Cc1cc(O[C@@H](C)CCC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 277 | 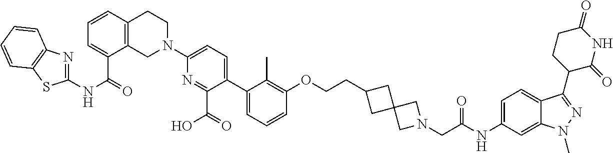 [SMILES: Cc1c(OCCC2CC3(C2)CN(CC(=O)Nc2ccc4c(C5CCC(=O)NC5=O)nn(C)c4c2)C3)cccc1-c1ccc(N2CCc3cccc(C(=O)Nc4nc5ccccc5s4)c3C2)nc1C(=O)O] |
| 278 | 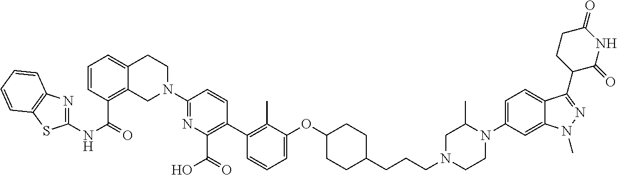 [SMILES: Cc1c(OC2CCC(CCCN3CCN(c4ccc5c(C6CCC(=O)NC6=O)nn(C)c5c4)C(C)C3)CC2)cccc1-c1ccc(N2CCc3cccc(C(=O)Nc4nc5ccccc5s4)c3C2)nc1C(=O)O] |
| 278a | 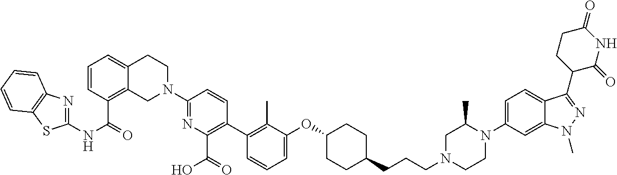 [SMILES: Cc1c(O[C@H]2CC[C@H](CCCN3CCN(c4ccc5c(C6CCC(=O)NC6=O)nn(C)c5c4)[C@H](C)C3)CC2)cccc1-c1ccc(N2CCc3cccc(C(=O)Nc4nc5ccccc5s4)c3C2)nc1C(=O)O] |
| 278b | 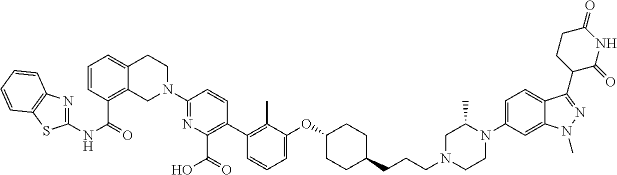 [SMILES: Cc1c(O[C@H]2CC[C@H](CCCN3CCN(c4ccc5c(C6CCC(=O)NC6=O)nn(C)c5c4)[C@@H](C)C3)CC2)cccc1-c1ccc(N2CCc3cccc(C(=O)Nc4nc5ccccc5s4)c3C2)nc1C(=O)O] |
| 279 | 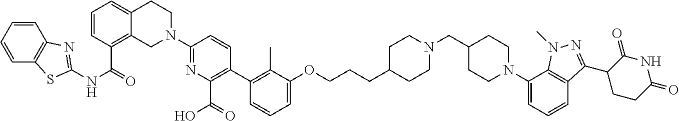 [SMILES: Cc1c(OCCCC2CCN(CC3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 280 | 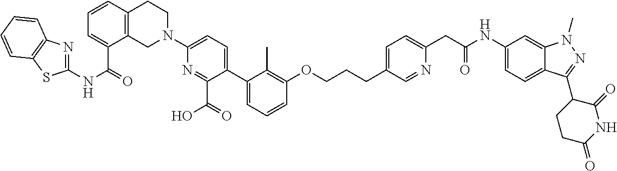 [SMILES: Cc1c(OCCCc2ccc(CC(=O)Nc3ccc4c(C5CCC(=O)NC5=O)nn(C)c4c3)nc2)cccc1-c1ccc(N2CCc3cccc(C(=O)Nc4nc5ccccc5s4)c3C2)nc1C(=O)O] |
| 281 | 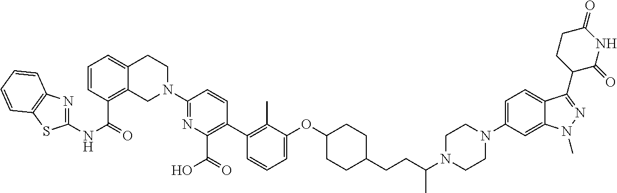 [SMILES: Cc1c(OC2CCC(CCC(C)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 281a | 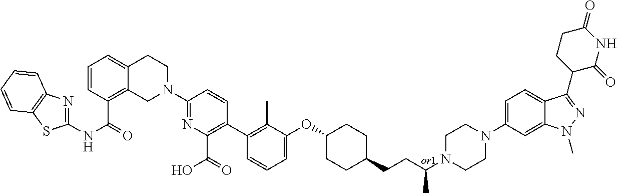 [SMILES: Cc1c(O[C@H]2CC[C@H](CC[C@H](C)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 281b | 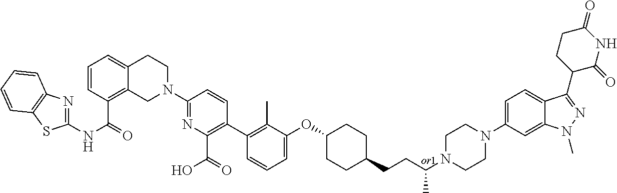 [SMILES: Cc1c(O[C@H]2CC[C@H](CC[C@@H](C)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 282 | 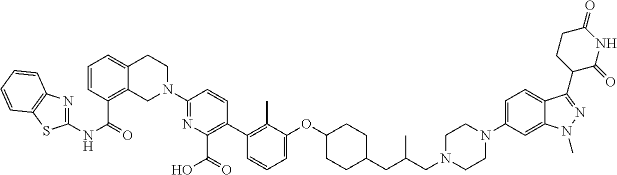 [SMILES: Cc1c(OC2CCC(CC(C)CN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 282a | 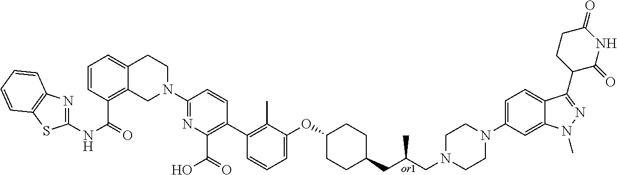 [SMILES: Cc1c(O[C@H]2CC[C@H](C[C@@H](C)CN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 282b | 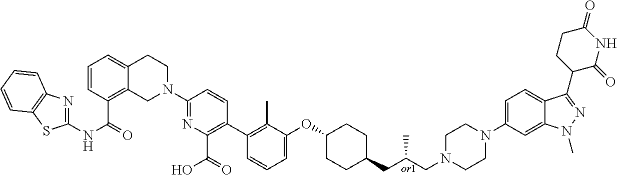 [SMILES: Cc1c(O[C@H]2CC[C@H](C[C@H](C)CN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 283 | 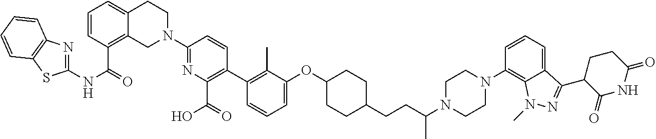 [SMILES: Cc1c(OC2CCC(CCC(C)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 283a | 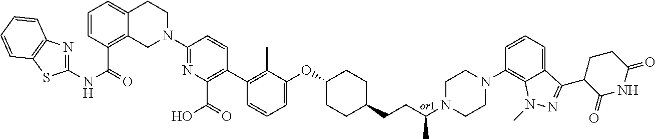 [SMILES: Cc1c(O[C@H]2CC[C@H](CC[C@H](C)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 283b | 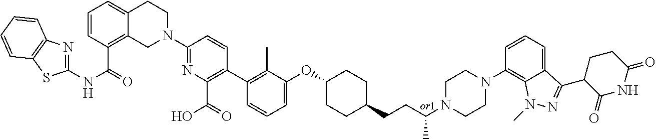 [SMILES: Cc1c(O[C@H]2CC[C@H](CC[C@@H](C)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 284 | 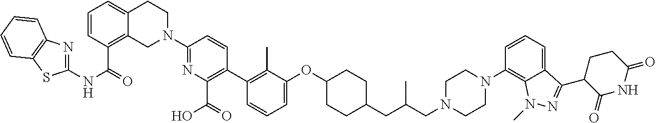 [SMILES: Cc1c(OC2CCC(CC(C)CN3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 284a | 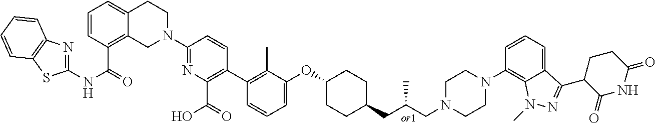 [SMILES: Cc1c(O[C@H]2CC[C@H](C[C@H](C)CN3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 284b | 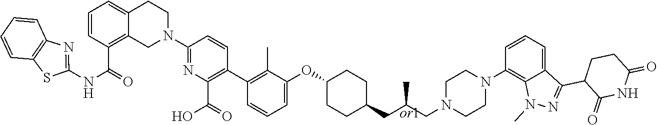 [SMILES: Cc1c(O[C@H]2CC[C@H](C[C@@H](C)CN3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 285 | 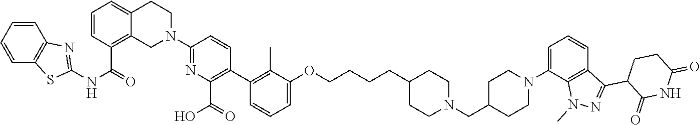 [SMILES: Cc1c(OCCCCC2CCN(CC3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 286 | 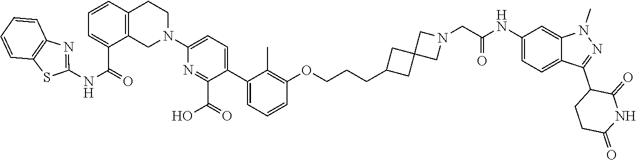 [SMILES: Cc1c(OCCCC2CC3(C2)CN(CC(=O)Nc2ccc4c(C5CCC(=O)NC5=O)nn(C)c4c2)C3)cccc1-c1ccc(N2CCc3cccc(C(=O)Nc4nc5ccccc5s4)c3C2)nc1C(=O)O] |
| 287 | 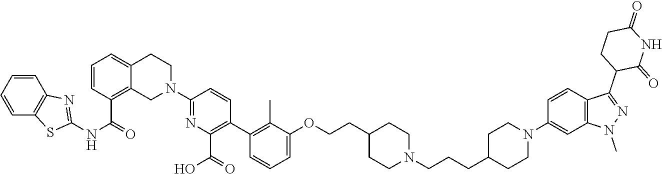 [SMILES: Cc1c(OCCC2CCN(CCCC3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 288 | 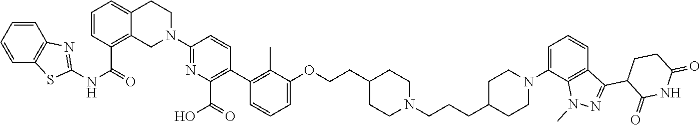 [SMILES: Cc1c(OCCC2CCN(CCCC3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 289 | 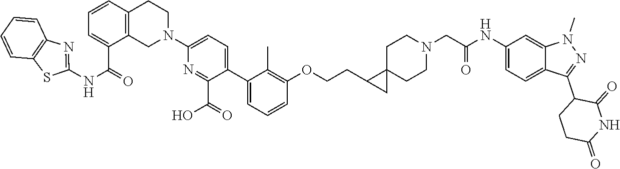 [SMILES: Cc1c(OCCC2CC23CCN(CC(=O)Nc2ccc4c(C5CCC(=O)NC5=O)nn(C)c4c2)CC3)cccc1-c1ccc(N2CCc3cccc(C(=O)Nc4nc5ccccc5s4)c3C2)nc1C(=O)O] |
| 289a | 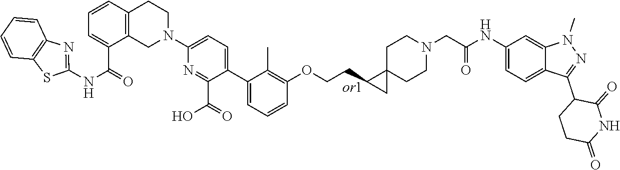 [SMILES: Cc1c(OCC[C@@H]2CC23CCN(CC(=O)Nc2ccc4c(C5CCC(=O)NC5=O)nn(C)c4c2)CC3)cccc1-c1ccc(N2CCc3cccc(C(=O)Nc4nc5ccccc5s4)c3C2)nc1C(=O)O] |
| 289b | 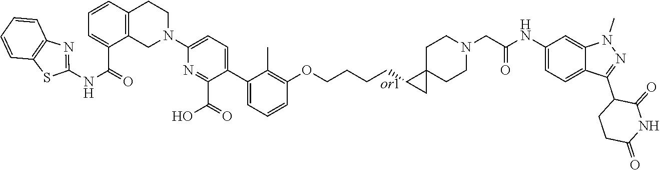 [SMILES: Cc1c(OCCCC[C@H]2CC23CCN(CC(=O)Nc2ccc4c(C5CCC(=O)NC5=O)nn(C)c4c2)CC3)cccc1-c1ccc(N2CCc3cccc(C(=O)Nc4nc5ccccc5s4)c3C2)nc1C(=O)O] |
| 290 | 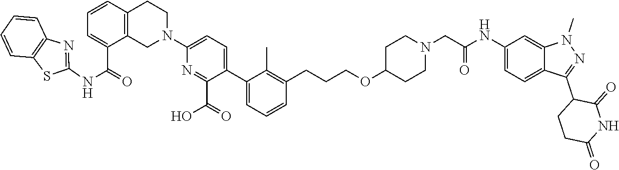 [SMILES: Cc1c(CCCOC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 291 | 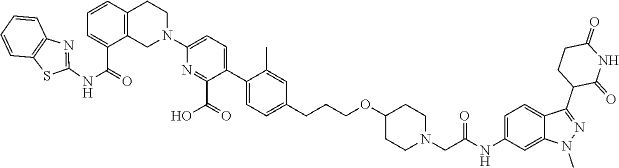 [SMILES: Cc1cc(CCCOC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 292 | 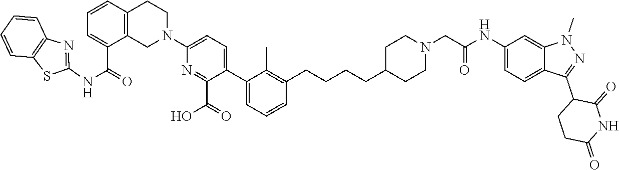 [SMILES: Cc1c(CCCCC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 293 | 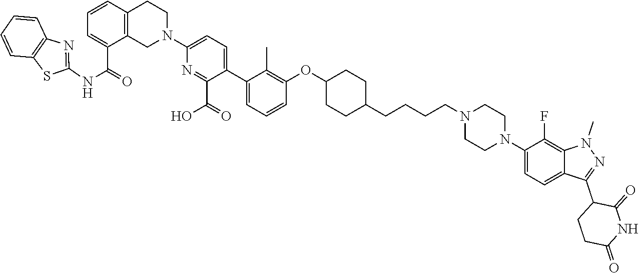 [SMILES: Cc1c(OC2CCC(CCCCN3CCN(c4ccc5c(C6CCC(=O)NC6=O)nn(C)c5c4F)CC3)CC2)cccc1-c1ccc(N2CCc3cccc(C(=O)Nc4nc5ccccc5s4)c3C2)nc1C(=O)O] |
| 293a | 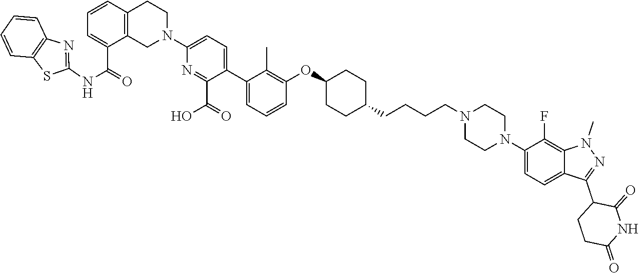 [SMILES: Cc1c(O[C@H]2CC[C@H](CCCCN3CCN(c4ccc5c(C6CCC(=O)NC6=O)nn(C)c5c4F)CC3)CC2)cccc1-c1ccc(N2CCc3cccc(C(=O)Nc4nc5ccccc5s4)c3C2)nc1C(=O)O] |
| 294 | 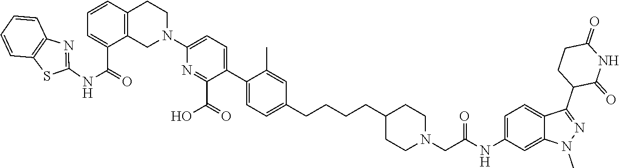 [SMILES: Cc1cc(CCCCC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 295 | 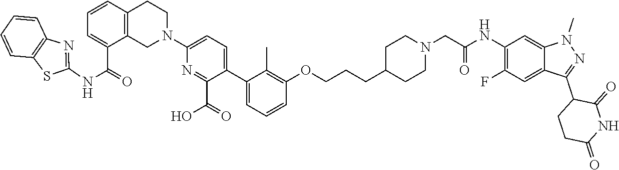 [SMILES: Cc1c(OCCCC2CCN(CC(=O)Nc3cc4c(cc3F)c(C3CCC(=O)NC3=O)nn4C)CC2)cccc1-c1ccc(N2CCc3cccc(C(=O)Nc4nc5ccccc5s4)c3C2)nc1C(=O)O] |
| 296 | 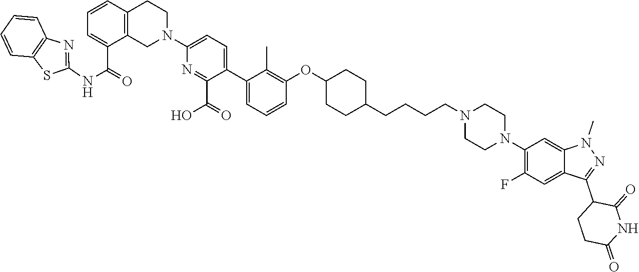 [SMILES: Cc1c(OC2CCC(CCCCN3CCN(c4cc5c(cc4F)c(C4CCC(=O)NC4=O)nn5C)CC3)CC2)cccc1-c1ccc(N2CCc3cccc(C(=O)Nc4nc5ccccc5s4)c3C2)nc1C(=O)O] |
| 296a | 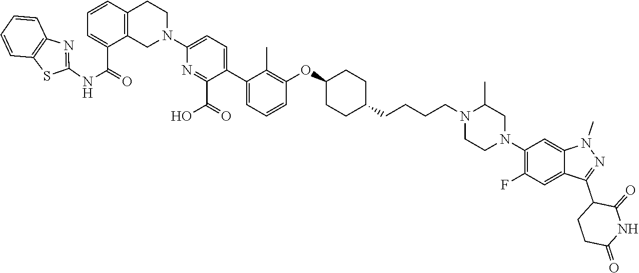 [SMILES: Cc1c(O[C@H]2CC[C@H](CCCCN3CCN(c4cc5c(cc4F)c(C4CCC(=O)NC4=O)nn5C)CC3C)CC2)cccc1-c1ccc(N2CCc3cccc(C(=O)Nc4nc5ccccc5s4)c3C2)nc1C(=O)O] |
| 297 | 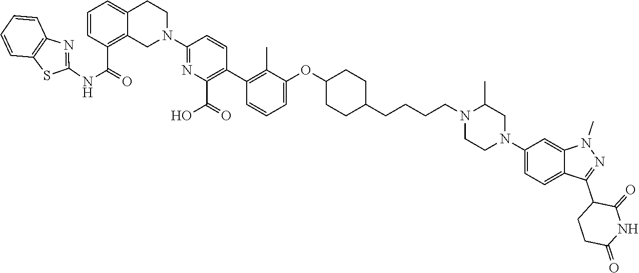 [SMILES: Cc1c(OC2CCC(CCCCN3CCN(c4ccc5c(C6CCC(=O)NC6=O)nn(C)c5c4)CC3C)CC2)cccc1-c1ccc(N2CCc3cccc(C(=O)Nc4nc5ccccc5s4)c3C2)nc1C(=O)O] |
| 297a | 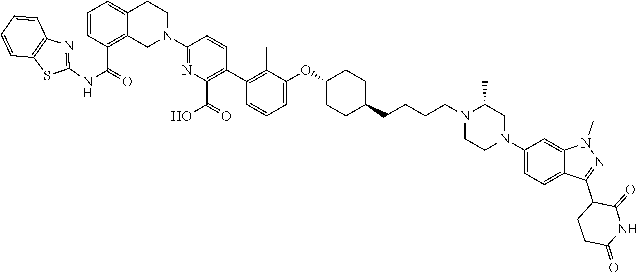 [SMILES: Cc1c(O[C@H]2CC[C@H](CCCCN3CCN(c4ccc5c(C6CCC(=O)NC6=O)nn(C)c5c4)C[C@H]3C)CC2)cccc1-c1ccc(N2CCc3cccc(C(=O)Nc4nc5ccccc5s4)c3C2)nc1C(=O)O] |
| 297b | 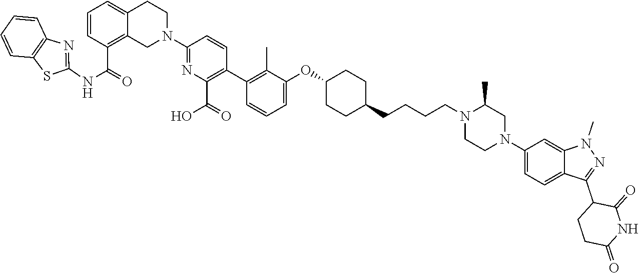 [SMILES: Cc1c(O[C@H]2CC[C@H](CCCCN3CCN(c4ccc5c(C6CCC(=O)NC6=O)nn(C)c5c4)C[C@@H]3C)CC2)cccc1-c1ccc(N2CCc3cccc(C(=O)Nc4nc5ccccc5s4)c3C2)nc1C(=O)O] |
| 298 | 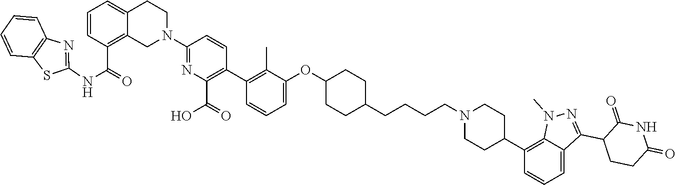 [SMILES: Cc1c(OC2CCC(CCCCN3CCC(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 298a | 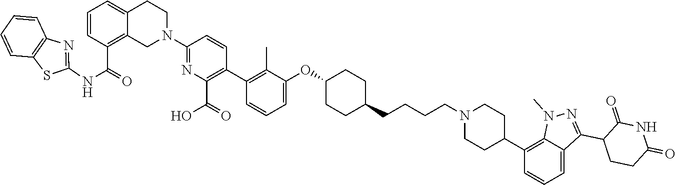 [SMILES: Cc1c(O[C@H]2CC[C@H](CCCCN3CCC(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 299 | 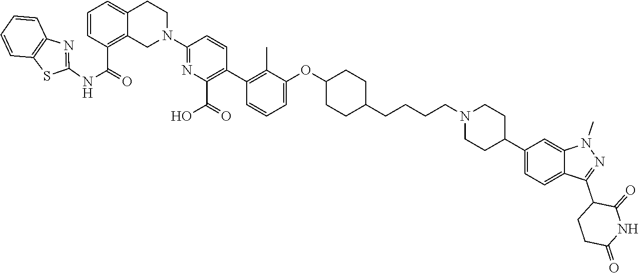 [SMILES: Cc1c(OC2CCC(CCCCN3CCC(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 299a | 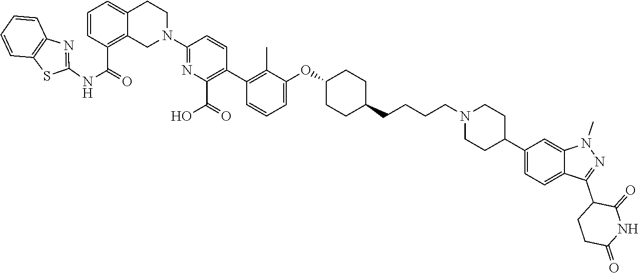 [SMILES: Cc1c(O[C@H]2CC[C@H](CCCCN3CCC(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 300 | 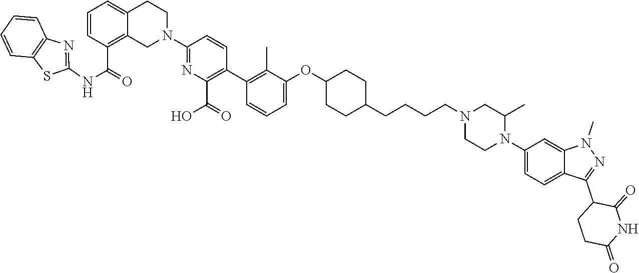 [SMILES: Cc1c(OC2CCC(CCCCN3CCN(c4ccc5c(C6CCC(=O)NC6=O)nn(C)c5c4)C(C)C3)CC2)cccc1-c1ccc(N2CCc3cccc(C(=O)Nc4nc5ccccc5s4)c3C2)nc1C(=O)O] |
| 300a | 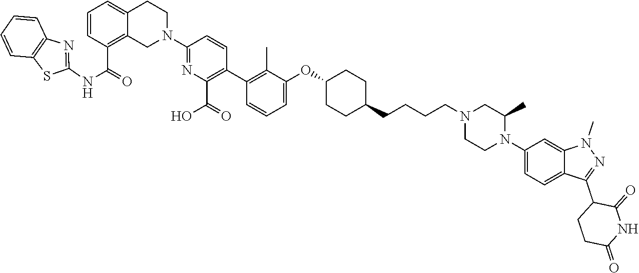 [SMILES: Cc1c(O[C@H]2CC[C@H](CCCCN3CCN(c4ccc5c(C6CCC(=O)NC6=O)nn(C)c5c4)[C@H](C)C3)CC2)cccc1-c1ccc(N2CCc3cccc(C(=O)Nc4nc5ccccc5s4)c3C2)nc1C(=O)O] |
| 300b | 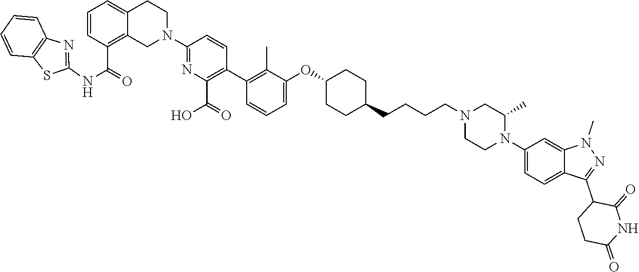 [SMILES: Cc1c(O[C@H]2CC[C@H](CCCCN3CCN(c4ccc5c(C6CCC(=O)NC6=O)nn(C)c5c4)[C@@H](C)C3)CC2)cccc1-c1ccc(N2CCc3cccc(C(=O)Nc4nc5ccccc5s4)c3C2)nc1C(=O)O] |
| 301 | 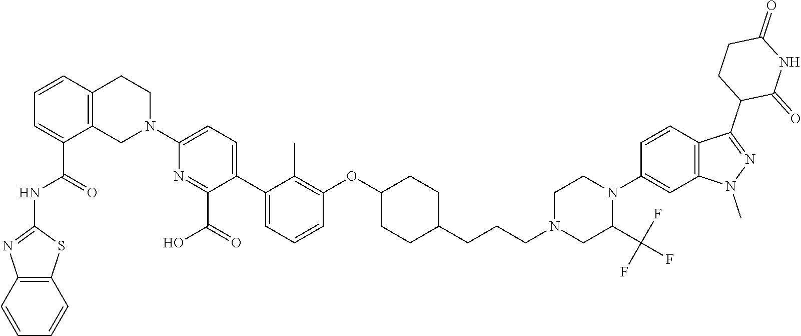 [SMILES: Cc1c(OC2CCC(CCCN3CCN(c4ccc5c(C6CCC(=O)NC6=O)nn(C)c5c4)C(C(F)(F)F)C3)CC2)cccc1-c1ccc(N2CCc3cccc(C(=O)Nc4nc5ccccc5s4)c3C2)nc1C(=O)O] |
| 301a | 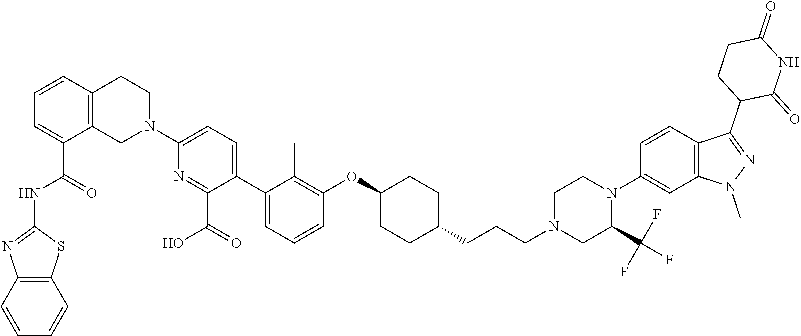 [SMILES: Cc1c(O[C@H]2CC[C@H](CCCN3CCN(c4ccc5c(C6CCC(=O)NC6=O)nn(C)c5c4)[C@@H](C(F)(F)F)C3)CC2)cccc1-c1ccc(N2CCc3cccc(C(=O)Nc4nc5ccccc5s4)c3C2)nc1C(=O)O] |
| 301b | 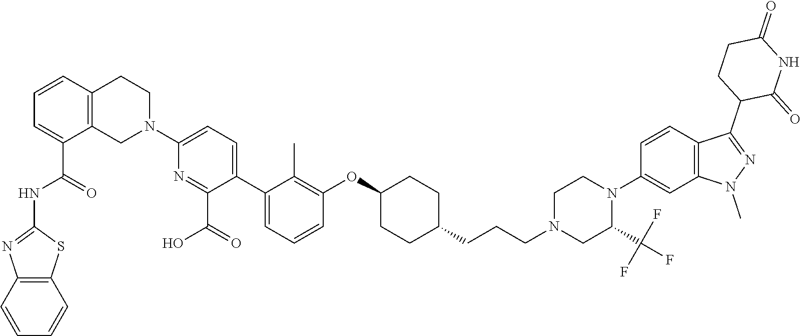 [SMILES: Cc1c(O[C@H]2CC[C@H](CCCN3CCN(c4ccc5c(C6CCC(=O)NC6=O)nn(C)c5c4)[C@H](C(F)(F)F)C3)CC2)cccc1-c1ccc(N2CCc3cccc(C(=O)Nc4nc5ccccc5s4)c3C2)nc1C(=O)O] |
| 302 | 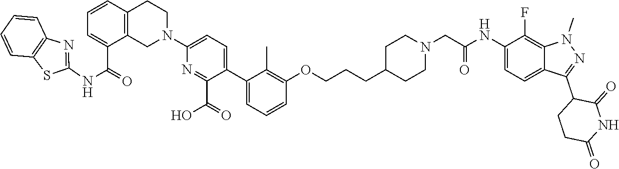 [SMILES: Cc1c(OCCCC2CCN(CC(=O)Nc3ccc4c(C5CCC(=O)NC5=O)nn(C)c4c3F)CC2)cccc1-c1ccc(N2CCc3cccc(C(=O)Nc4nc5ccccc5s4)c3C2)nc1C(=O)O] |
| 303 | 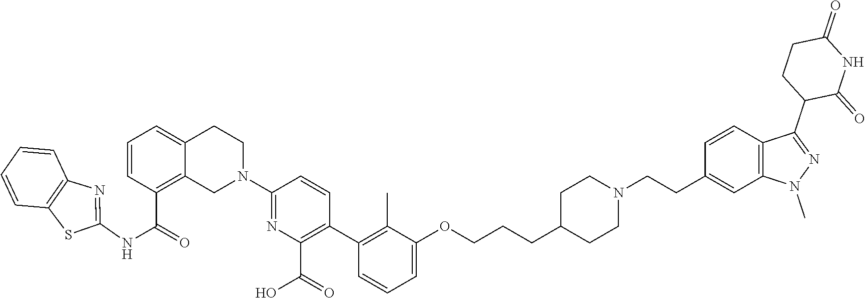 [SMILES: Cc1c(OCCCC2CCN(CCc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 304 | 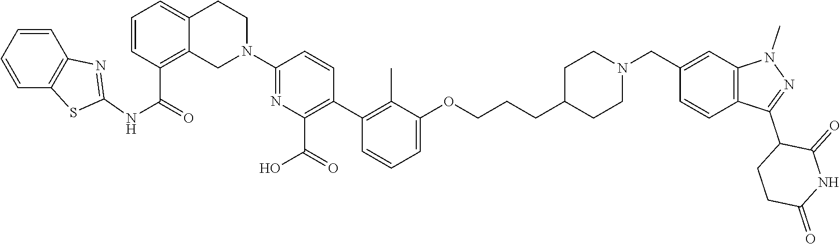 [SMILES: Cc1c(OCCCC2CCN(Cc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 305 | 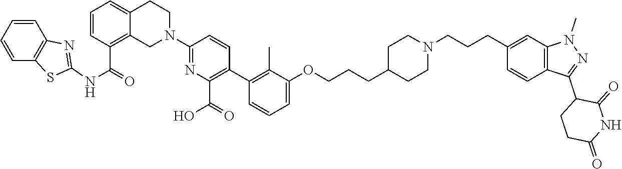 [SMILES: Cc1c(OCCCC2CCN(CCCc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 306 | 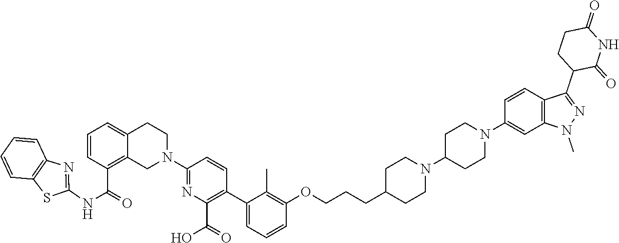 [SMILES: Cc1c(OCCCC2CCN(C3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 307 | 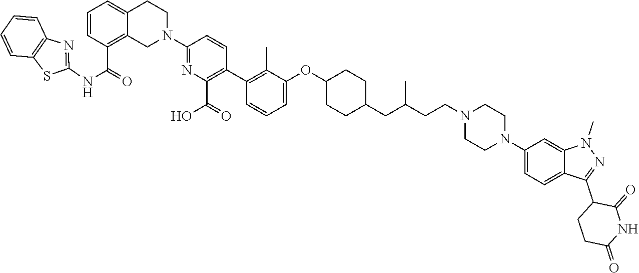 [SMILES: Cc1c(OC2CCC(CC(C)CCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 307a | 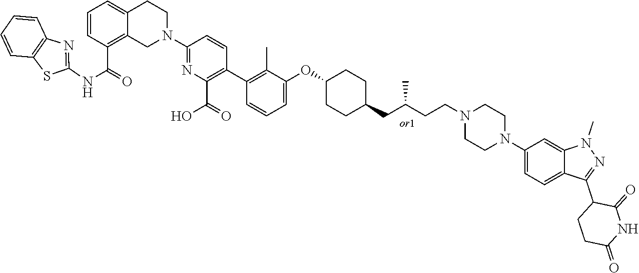 [SMILES: Cc1c(O[C@H]2CC[C@H](C[C@H](C)CCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 307b | 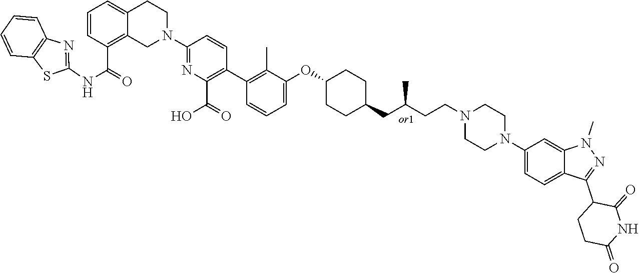 [SMILES: Cc1c(O[C@H]2CC[C@H](C[C@@H](C)CCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 308 | 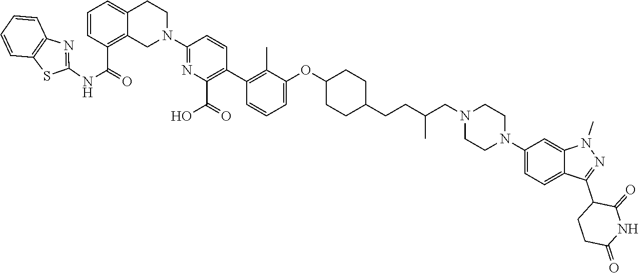 [SMILES: Cc1c(OC2CCC(CCC(C)CN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 308a | 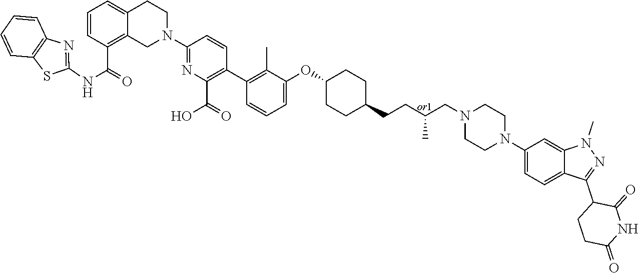 [SMILES: Cc1c(O[C@H]2CC[C@H](CC[C@@H](C)CN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 308b | 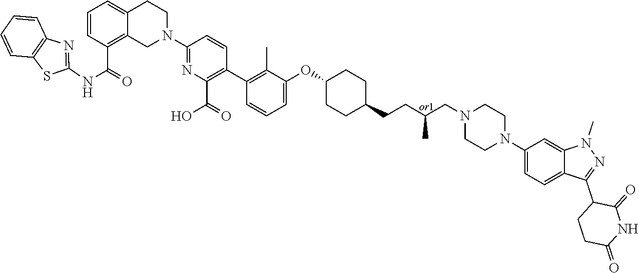 [SMILES: Cc1c(O[C@H]2CC[C@H](CC[C@H](C)CN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 309 | 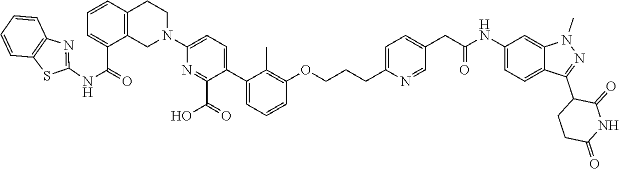 [SMILES: Cc1c(OCCCc2ccc(CC(=O)Nc3ccc4c(C5CCC(=O)NC5=O)nn(C)c4c3)cn2)cccc1-c1ccc(N2CCc3cccc(C(=O)Nc4nc5ccccc5s4)c3C2)nc1C(=O)O] |
| 310 | 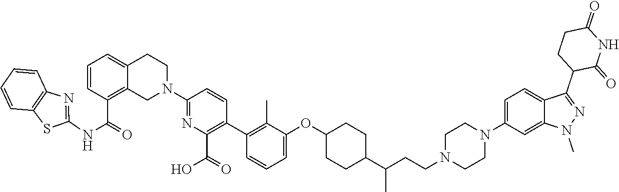 [SMILES: Cc1c(OC2CCC(C(C)CCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 310a | 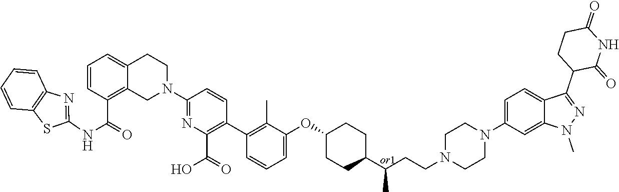 [SMILES: Cc1c(O[C@H]2CC[C@H]([C@H](C)CCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 310b | 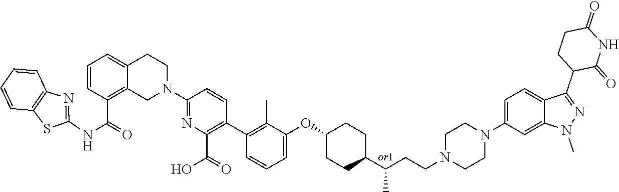 [SMILES: Cc1c(O[C@H]2CC[C@H]([C@@H](C)CCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 311 | 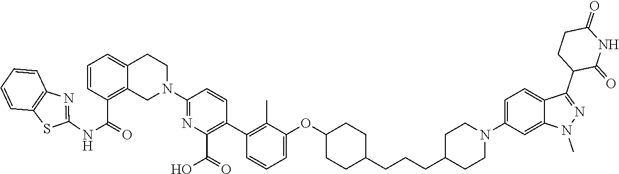 [SMILES: Cc1c(OC2CCC(CCCC3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 311a | 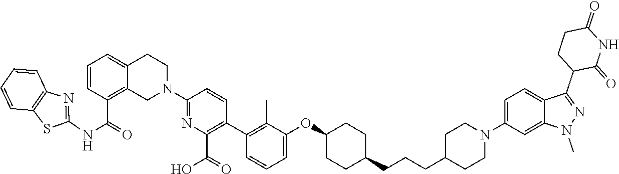 [SMILES: Cc1c(O[C@H]2CC[C@@H](CCCC3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 311b | 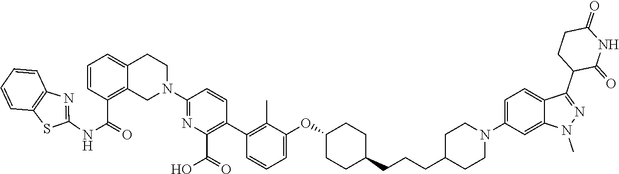 [SMILES: Cc1c(O[C@H]2CC[C@H](CCCC3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 312 | 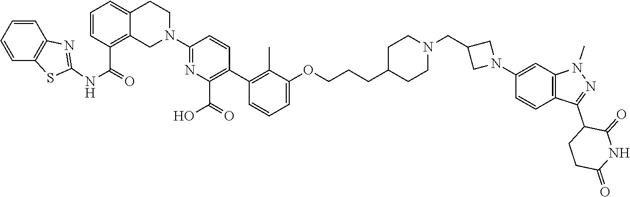 [SMILES: Cc1c(OCCCC2CCN(CC3CN(c4ccc5c(C6CCC(=O)NC6=O)nn(C)c5c4)C3)CC2)cccc1-c1ccc(N2CCc3cccc(C(=O)Nc4nc5ccccc5s4)c3C2)nc1C(=O)O] |
| 313 | 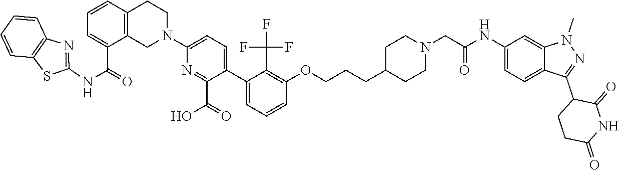 [SMILES: Cn1nc(C2CCC(=O)NC2=O)c2ccc(NC(=O)CN3CCC(CCCOc4cccc(-c5ccc(N6CCc7cccc(C(=O)Nc8nc9ccccc9s8)c7C6)nc5C(=O)O)c4C(F)(F)F)CC3)cc21] |
| 314 | 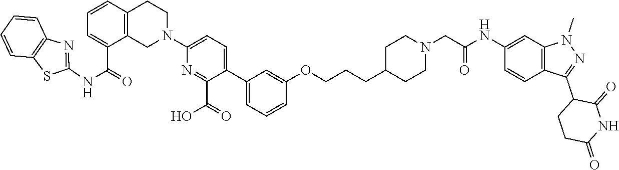 [SMILES: Cn1nc(C2CCC(=O)NC2=O)c2ccc(NC(=O)CN3CCC(CCCOc4cccc(-c5ccc(N6CCc7cccc(C(=O)Nc8nc9ccccc9s8)c7C6)nc5C(=O)O)c4)CC3)cc21] |
| 316 | 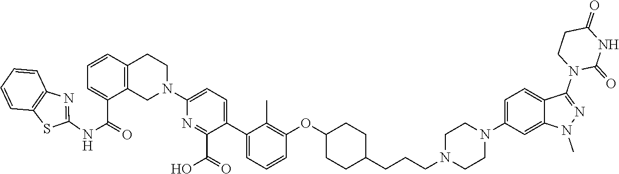 [SMILES: Cc1c(OC2CCC(CCCN3CCN(c4ccc5c(N6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 316a | 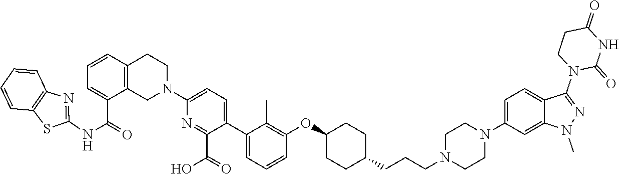 [SMILES: Cc1c(O[C@H]2CC[C@H](CCCN3CCN(c4ccc5c(N6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 317 | 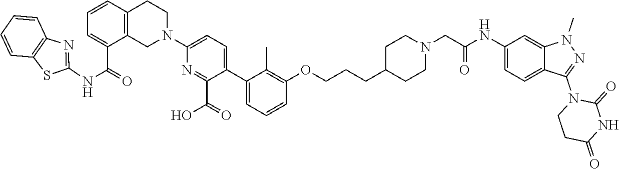 [SMILES: Cc1c(OCCCC2CCN(CC(=O)Nc3ccc4c(N5CCC(=O)NC5=O)nn(C)c4c3)CC2)cccc1-c1ccc(N2CCc3cccc(C(=O)Nc4nc5ccccc5s4)c3C2)nc1C(=O)O] |
| 318 | 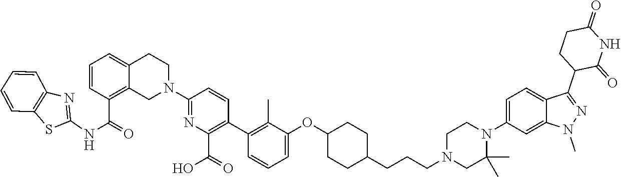 [SMILES: Cc1c(OC2CCC(CCCN3CCN(c4ccc5c(C6CCC(=O)NC6=O)nn(C)c5c4)C(C)(C)C3)CC2)cccc1-c1ccc(N2CCc3cccc(C(=O)Nc4nc5ccccc5s4)c3C2)nc1C(=O)O] |
| 318a | 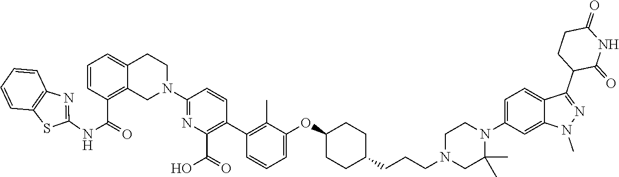 [SMILES: Cc1c(O[C@H]2CC[C@H](CCCN3CCN(c4ccc5c(C6CCC(=O)NC6=O)nn(C)c5c4)C(C)(C)C3)CC2)cccc1-c1ccc(N2CCc3cccc(C(=O)Nc4nc5ccccc5s4)c3C2)nc1C(=O)O] |
| 319 | 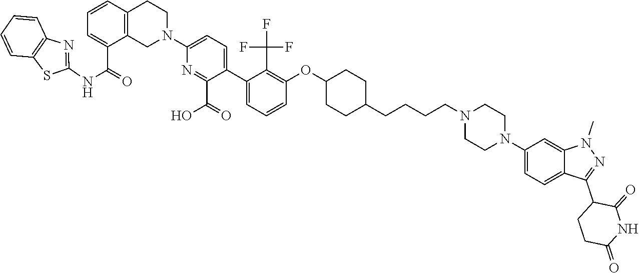 [SMILES: Cn1nc(C2CCC(=O)NC2=O)c2ccc(N3CCN(CCCCC4CCC(Oc5cccc(-c6ccc(N7CCc8cccc(C(=O)Nc9nc%10ccccc%10s9)c8C7)nc6C(=O)O)c5C(F)(F)F)CC4)CC3)cc21] |
| 319a | 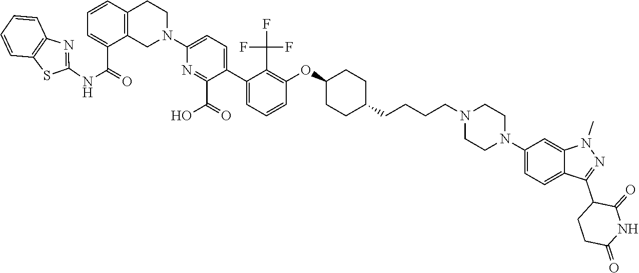 [SMILES: Cn1nc(C2CCC(=O)NC2=O)c2ccc(N3CCN(CCCC[C@H]4CC[C@H](Oc5cccc(-c6ccc(N7CCc8cccc(C(=O)Nc9nc%10ccccc%10s9)c8C7)nc6C(=O)O)c5C(F)(F)F)CC4)CC3)cc21] |
| 320 | 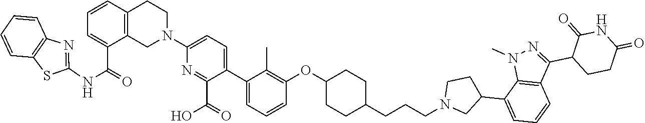 [SMILES: Cc1c(OC2CCC(CCCN3CCC(c4cccc5c(C6CCC(=O)NC6=O)nn(C)c45)C3)CC2)cccc1-c1ccc(N2CCc3cccc(C(=O)Nc4nc5ccccc5s4)c3C2)nc1C(=O)O] |
| 320a | 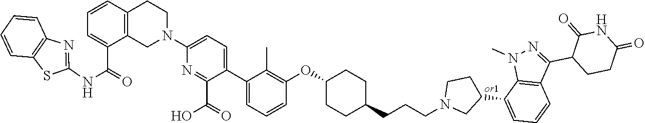 |
| 320b | 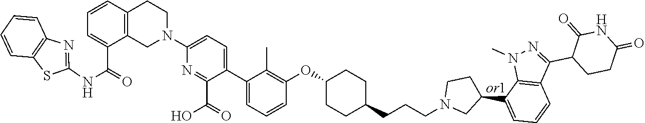 [SMILES: Cc1c(O[C@H]2CC[C@H](CCCN3CC[C@@H](c4cccc5c(C6CCC(=O)NC6=O)nn(C)c45)C3)CC2)cccc1-c1ccc(N2CCc3cccc(C(=O)Nc4nc5ccccc5s4)c3C2)nc1C(=O)O] |
| 321 | 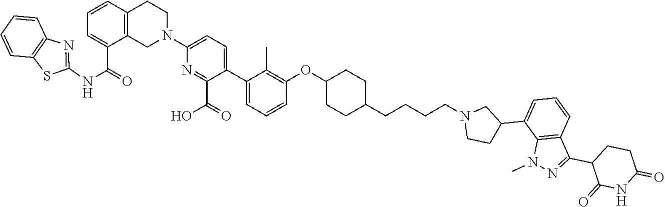 [SMILES: Cc1c(OC2CCC(CCCCN3CCC(c4cccc5c(C6CCC(=O)NC6=O)nn(C)c45)C3)CC2)cccc1-c1ccc(N2CCc3cccc(C(=O)Nc4nc5ccccc5s4)c3C2)nc1C(=O)O] |
| 321a | 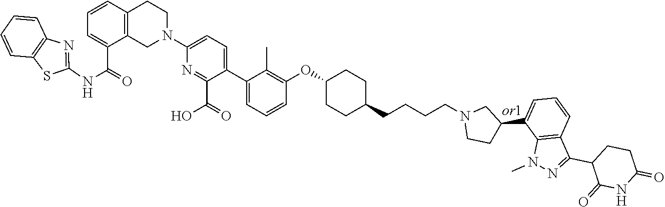 [SMILES: Cc1c(O[C@H]2CC[C@H](CCCCN3CC[C@H](c4cccc5c(C6CCC(=O)NC6=O)nn(C)c45)C3)CC2)cccc1-c1ccc(N2CCc3cccc(C(=O)Nc4nc5ccccc5s4)c3C2)nc1C(=O)O] |
| 321b | 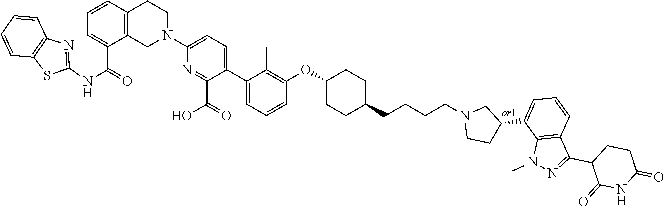 [SMILES: Cc1c(O[C@H]2CC[C@H](CCCCN3CC[C@@H](c4cccc5c(C6CCC(=O)NC6=O)nn(C)c45)C3)CC2)cccc1-c1ccc(N2CCc3cccc(C(=O)Nc4nc5ccccc5s4)c3C2)nc1C(=O)O] |
| 322 | 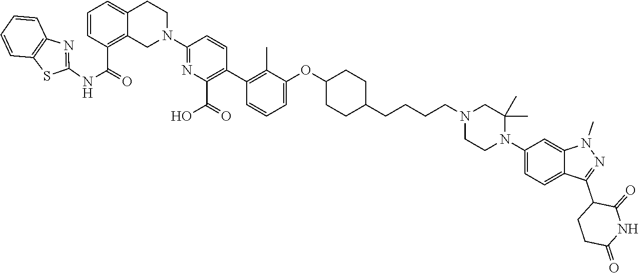 [SMILES: Cc1c(OC2CCC(CCCCN3CCN(c4ccc5c(C6CCC(=O)NC6=O)nn(C)c5c4)C(C)(C)C3)CC2)cccc1-c1ccc(N2CCc3cccc(C(=O)Nc4nc5ccccc5s4)c3C2)nc1C(=O)O] |
| 322a | 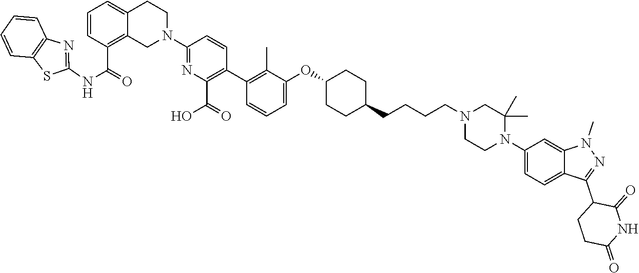 [SMILES: Cc1c(O[C@H]2CC[C@H](CCCCN3CCN(c4ccc5c(C6CCC(=O)NC6=O)nn(C)c5c4)C(C)(C)C3)CC2)cccc1-c1ccc(N2CCc3cccc(C(=O)Nc4nc5ccccc5s4)c3C2)nc1C(=O)O] |
| 323 | 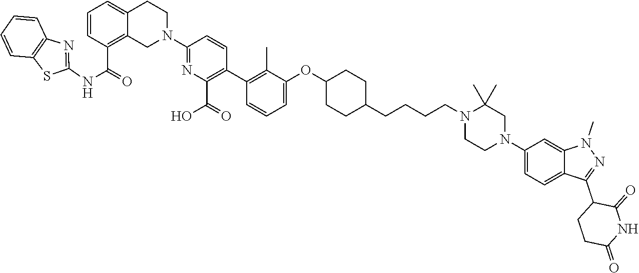 [SMILES: Cc1c(OC2CCC(CCCCN3CCN(c4ccc5c(C6CCC(=O)NC6=O)nn(C)c5c4)CC3(C)C)CC2)cccc1-c1ccc(N2CCc3cccc(C(=O)Nc4nc5ccccc5s4)c3C2)nc1C(=O)O] |
| 323a | 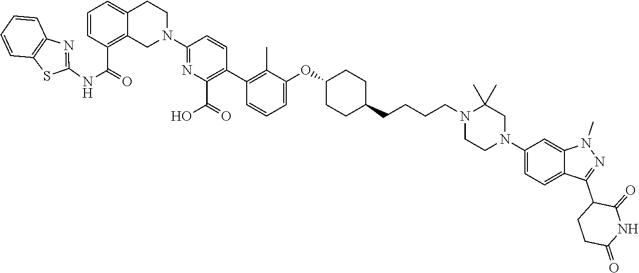 [SMILES: Cc1c(O[C@H]2CC[C@H](CCCCN3CCN(c4ccc5c(C6CCC(=O)NC6=O)nn(C)c5c4)CC3(C)C)CC2)cccc1-c1ccc(N2CCc3cccc(C(=O)Nc4nc5ccccc5s4)c3C2)nc1C(=O)O] |
| 324 | 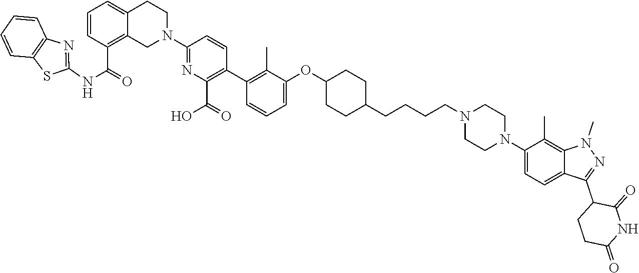 [SMILES: Cc1c(OC2CCC(CCCCN3CCN(c4ccc5c(C6CCC(=O)NC6=O)nn(C)c5c4C)CC3)CC2)cccc1-c1ccc(N2CCc3cccc(C(=O)Nc4nc5ccccc5s4)c3C2)nc1C(=O)O] |
| 324a | 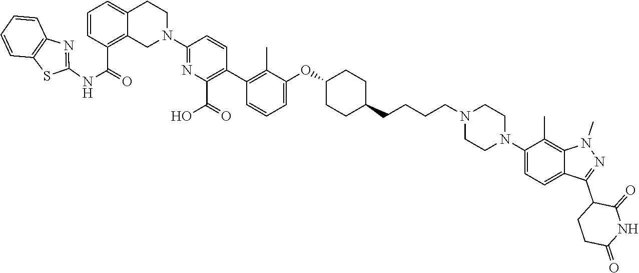 [SMILES: Cc1c(O[C@H]2CC[C@H](CCCCN3CCN(c4ccc5c(C6CCC(=O)NC6=O)nn(C)c5c4C)CC3)CC2)cccc1-c1ccc(N2CCc3cccc(C(=O)Nc4nc5ccccc5s4)c3C2)nc1C(=O)O] |
| 325 | 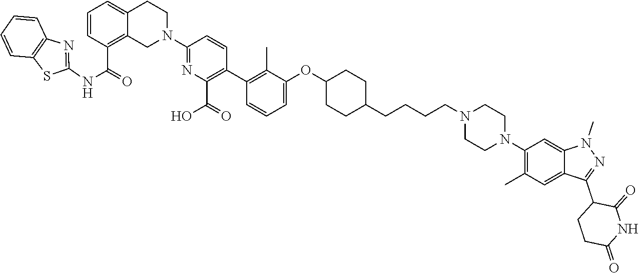 [SMILES: Cc1cc2c(C3CCC(=O)NC3=O)nn(C)c2cc1N1CCN(CCCCC2CCC(Oc3cccc(-c4ccc(N5CCc6cccc(C(=O)Nc7nc8ccccc8s7)c6C5)nc4C(=O)O)c3C)CC2)CC1] |
| 325a | 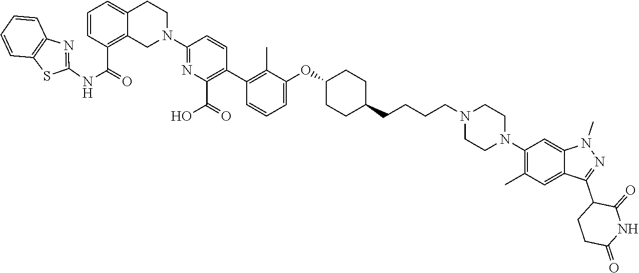 [SMILES: Cc1cc2c(C3CCC(=O)NC3=O)nn(C)c2cc1N1CCN(CCCC[C@H]2CC[C@H](Oc3cccc(-c4ccc(N5CCc6cccc(C(=O)Nc7nc8ccccc8s7)c6C5)nc4C(=O)O)c3C)CC2)CC1] |
| 326 | 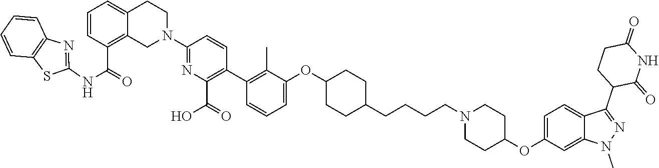 [SMILES: Cc1c(OC2CCC(CCCCN3CCC(Oc4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 326a | 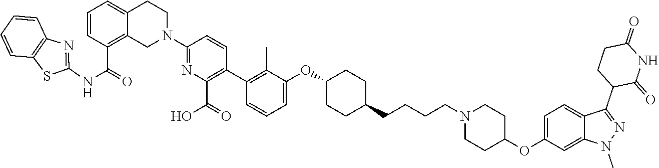 [SMILES: Cc1c(O[C@H]2CC[C@H](CCCCN3CCC(Oc4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 327 | 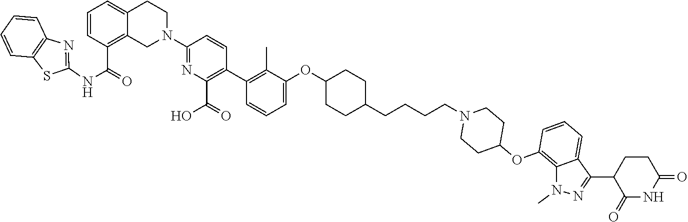 [SMILES: Cc1c(OC2CCC(CCCCN3CCC(Oc4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 327a | 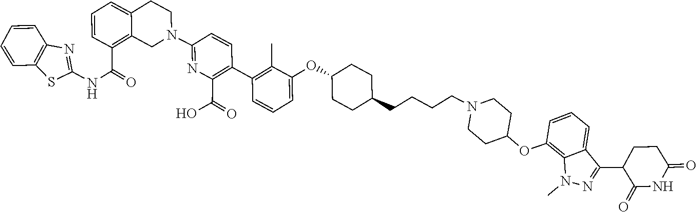 [SMILES: Cc1c(O[C@H]2CC[C@H](CCCCN3CCC(Oc4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 328 | 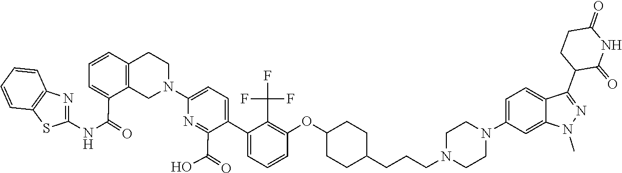 [SMILES: Cn1nc(C2CCC(=O)NC2=O)c2ccc(N3CCN(CCCC4CCC(Oc5cccc(-c6ccc(N7CCc8cccc(C(=O)Nc9nc%10ccccc%10s9)c8C7)nc6C(=O)O)c5C(F)(F)F)CC4)CC3)cc21] |
| 328a | 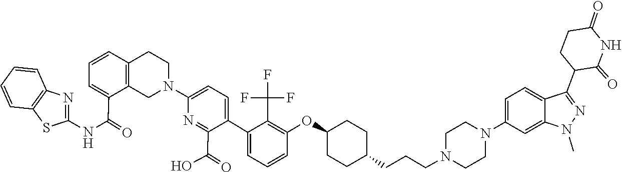 [SMILES: Cn1nc(C2CCC(=O)NC2=O)c2ccc(N3CCN(CCC[C@H]4CC[C@H](Oc5cccc(-c6ccc(N7CCc8cccc(C(=O)Nc9nc%10ccccc%10s9)c8C7)nc6C(=O)O)c5C(F)(F)F)CC4)CC3)cc21] |
| 329 | 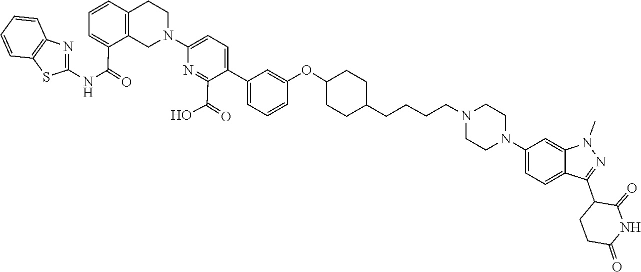 [SMILES: Cn1nc(C2CCC(=O)NC2=O)c2ccc(N3CCN(CCCCC4CCC(Oc5cccc(-c6ccc(N7CCc8cccc(C(=O)Nc9nc%10ccccc%10s9)c8C7)nc6C(=O)O)c5)CC4)CC3)cc21] |
| 329a | 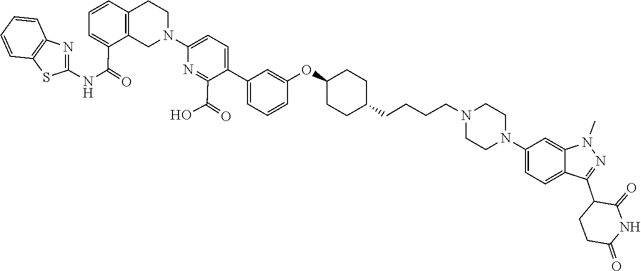 [SMILES: Cn1nc(C2CCC(=O)NC2=O)c2ccc(N3CCN(CCCC[C@H]4CC[C@H](Oc5cccc(-c6ccc(N7CCc8cccc(C(=O)Nc9nc%10ccccc%10s9)c8C7)nc6C(=O)O)c5)CC4)CC3)cc21] |
| 330 | 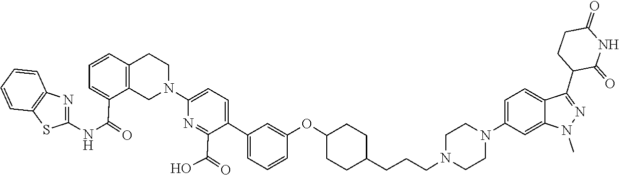 [SMILES: Cn1nc(C2CCC(=O)NC2=O)c2ccc(N3CCN(CCCC4CCC(Oc5cccc(-c6ccc(N7CCc8cccc(C(=O)Nc9nc%10ccccc%10s9)c8C7)nc6C(=O)O)c5)CC4)CC3)cc21] |
| 330a | 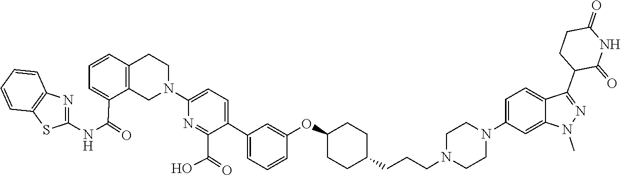 [SMILES: Cn1nc(C2CCC(=O)NC2=O)c2ccc(N3CCN(CCC[C@H]4CC[C@H](Oc5cccc(-c6ccc(N7CCc8cccc(C(=O)Nc9nc%10ccccc%10s9)c8C7)nc6C(=O)O)c5)CC4)CC3)cc21] |
| 331 | 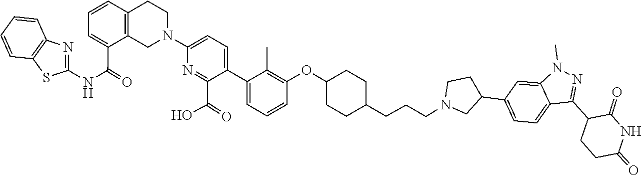 [SMILES: Cc1c(OC2CCC(CCCN3CCC(c4ccc5c(C6CCC(=O)NC6=O)nn(C)c5c4)C3)CC2)cccc1-c1ccc(N2CCc3cccc(C(=O)Nc4nc5ccccc5s4)c3C2)nc1C(=O)O] |
| 331a | 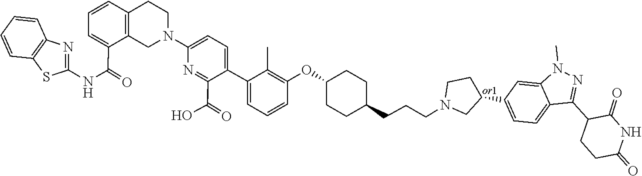 [SMILES: Cc1c(O[C@H]2CC[C@H](CCCN3CC[C@H](c4ccc5c(C6CCC(=O)NC6=O)nn(C)c5c4)C3)CC2)cccc1-c1ccc(N2CCc3cccc(C(=O)Nc4nc5ccccc5s4)c3C2)nc1C(=O)O] |
| 331b | 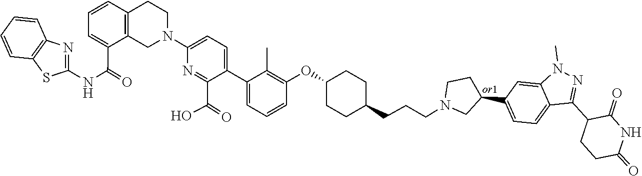 [SMILES: Cc1c(O[C@H]2CC[C@H](CCCN3CC[C@@H](c4ccc5c(C6CCC(=O)NC6=O)nn(C)c5c4)C3)CC2)cccc1-c1ccc(N2CCc3cccc(C(=O)Nc4nc5ccccc5s4)c3C2)nc1C(=O)O] |
| 332 | 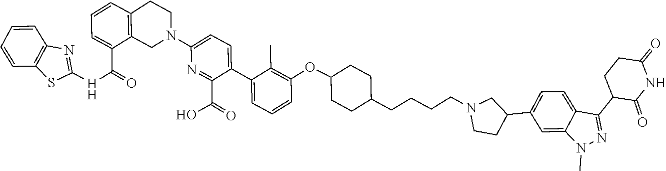 |
| 332a | 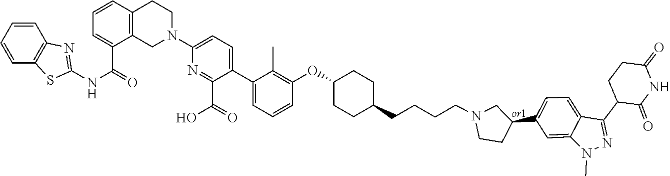 [SMILES: Cc1c(O[C@H]2CC[C@H](CCCCN3CC[C@H](c4ccc5c(C6CCC(=O)NC6=O)nn(C)c5c4)C3)CC2)cccc1-c1ccc(N2CCc3cccc(C(=O)Nc4nc5ccccc5s4)c3C2)nc1C(=O)O] |
| 332b | 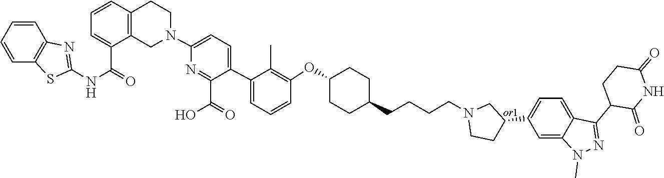 [SMILES: Cc1c(O[C@H]2CC[C@H](CCCCN3CC[C@@H](c4ccc5c(C6CCC(=O)NC6=O)nn(C)c5c4)C3)CC2)cccc1-c1ccc(N2CCc3cccc(C(=O)Nc4nc5ccccc5s4)c3C2)nc1C(=O)O] |
| 333 | 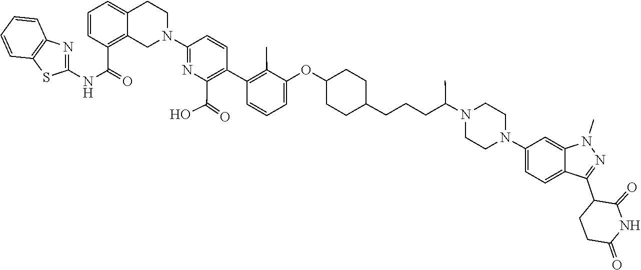 [SMILES: Cc1c(OC2CCC(CCCC(C)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 333a | 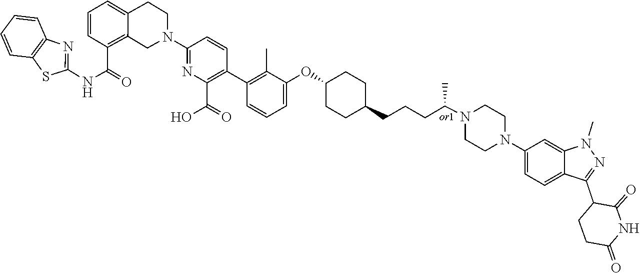 |
| 333b | 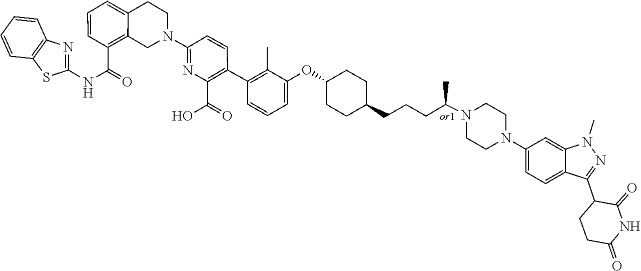 |
| 334 | 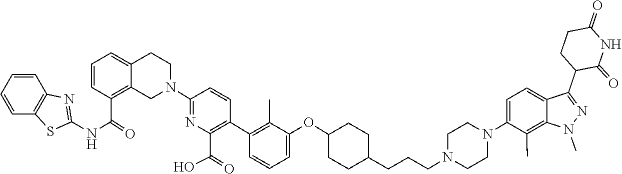 [SMILES: Cc1c(OC2CCC(CCCN3CCN(c4ccc5c(C6CCC(=O)NC6=O)nn(C)c5c4C)CC3)CC2)cccc1-c1ccc(N2CCc3cccc(C(=O)Nc4nc5ccccc5s4)c3C2)nc1C(=O)O] |
| 334a | 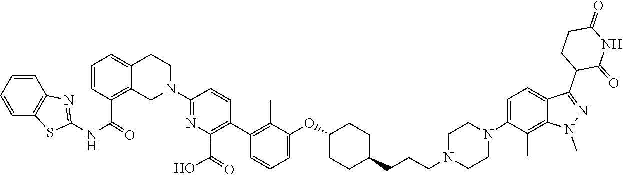 [SMILES: Cc1c(O[C@H]2CC[C@H](CCCN3CCN(c4ccc5c(C6CCC(=O)NC6=O)nn(C)c5c4C)CC3)CC2)cccc1-c1ccc(N2CCc3cccc(C(=O)Nc4nc5ccccc5s4)c3C2)nc1C(=O)O] |
| 335 | 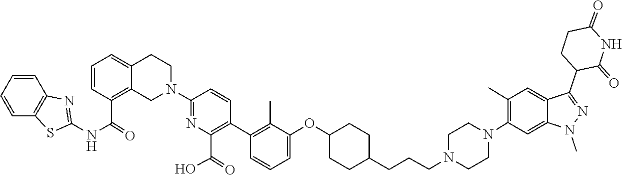 [SMILES: Cc1cc2c(C3CCC(=O)NC3=O)nn(C)c2cc1N1CCN(CCCC2CCC(Oc3cccc(-c4ccc(N5CCc6cccc(C(=O)Nc7nc8ccccc8s7)c6C5)nc4C(=O)O)c3C)CC2)CC1] |
| 335a | 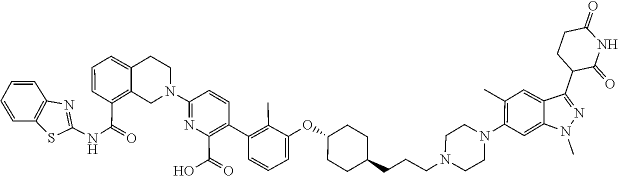 [SMILES: Cc1cc2c(C3CCC(=O)NC3=O)nn(C)c2cc1N1CCN(CCC[C@H]2CC[C@H](Oc3cccc(-c4ccc(N5CCc6cccc(C(=O)Nc7nc8ccccc8s7)c6C5)nc4C(=O)O)c3C)CC2)CC1] |
| 336 | 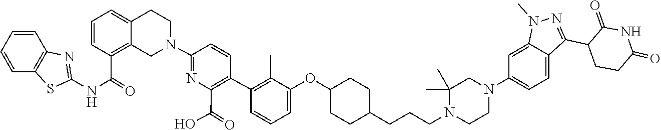 [SMILES: Cc1c(OC2CCC(CCCN3CCN(c4ccc5c(C6CCC(=O)NC6=O)nn(C)c5c4)CC3(C)C)CC2)cccc1-c1ccc(N2CCc3cccc(C(=O)Nc4nc5ccccc5s4)c3C2)nc1C(=O)O] |
| 336a | 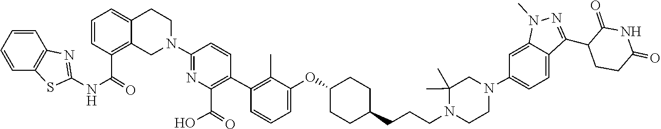 [SMILES: Cc1c(O[C@H]2CC[C@H](CCCN3CCN(c4ccc5c(C6CCC(=O)NC6=O)nn(C)c5c4)CC3(C)C)CC2)cccc1-c1ccc(N2CCc3cccc(C(=O)Nc4nc5ccccc5s4)c3C2)nc1C(=O)O] |
| 337 | 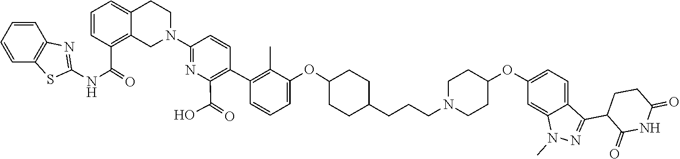 [SMILES: Cc1c(OC2CCC(CCCN3CCC(Oc4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 337a | 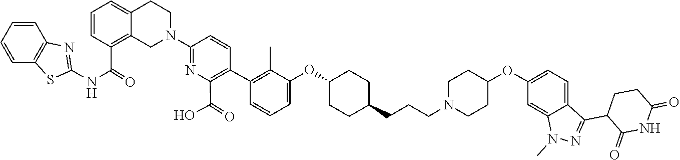 [SMILES: Cc1c(O[C@H]2CC[C@H](CCCN3CCC(Oc4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 338 | 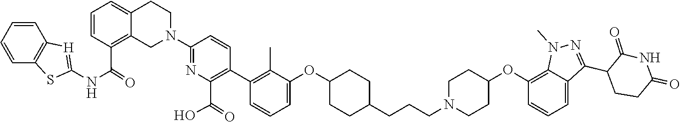 |
| 338a | 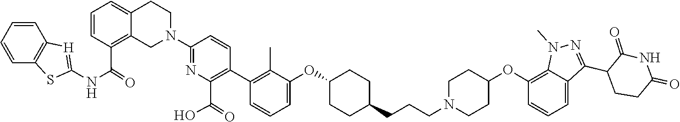 |
| 339 | 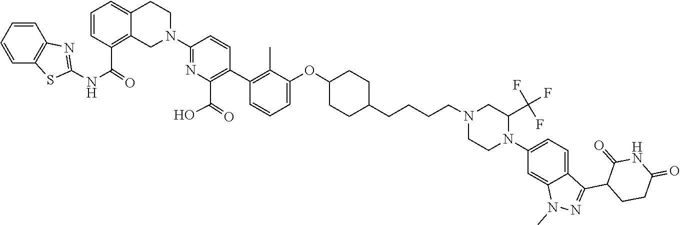 [SMILES: Cc1c(OC2CCC(CCCCN3CCN(c4ccc5c(C6CCC(=O)NC6=O)nn(C)c5c4)C(C(F)(F)F)C3)CC2)cccc1-c1ccc(N2CCc3cccc(C(=O)Nc4nc5ccccc5s4)c3C2)nc1C(=O)O] |
| 339a | 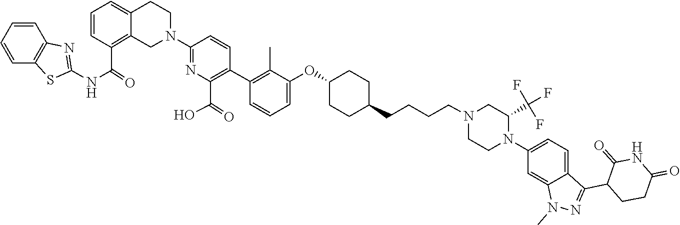 [SMILES: Cc1c(O[C@H]2CC[C@H](CCCCN3CCN(c4ccc5c(C6CCC(=O)NC6=O)nn(C)c5c4)[C@@H](C(F)(F)F)C3)CC2)cccc1-c1ccc(N2CCc3cccc(C(=O)Nc4nc5ccccc5s4)c3C2)nc1C(=O)O] |
| 339b | 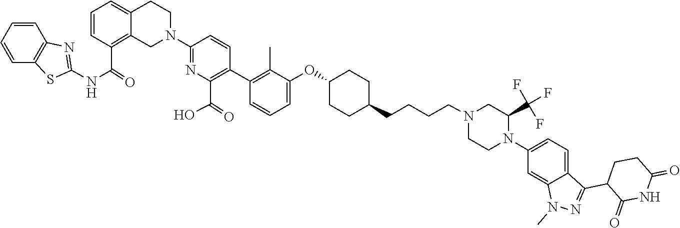 [SMILES: Cc1c(O[C@H]2CC[C@H](CCCCN3CCN(c4ccc5c(C6CCC(=O)NC6=O)nn(C)c5c4)[C@H](C(F)(F)F)C3)CC2)cccc1-c1ccc(N2CCc3cccc(C(=O)Nc4nc5ccccc5s4)c3C2)nc1C(=O)O] |
| 340 | 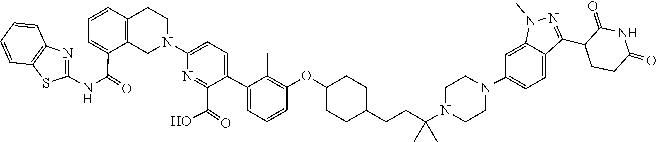 [SMILES: Cc1c(OC2CCC(CCC(C)(C)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 340a | 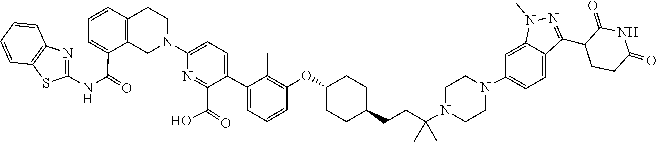 [SMILES: Cc1c(O[C@H]2CC[C@H](CCC(C)(C)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 341 | 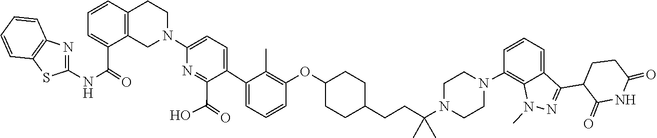 [SMILES: Cc1c(OC2CCC(CCC(C)(C)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 341a | 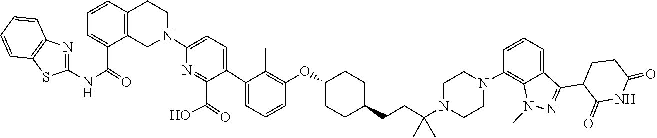 [SMILES: Cc1c(O[C@H]2CC[C@H](CCC(C)(C)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 342 | 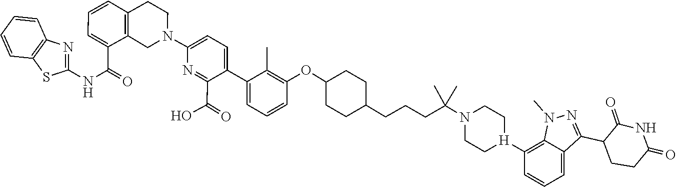 |
| 342a | 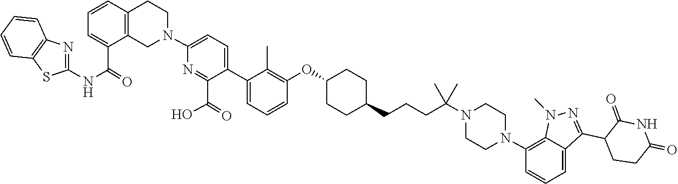 [SMILES: Cc1c(O[C@H]2CC[C@H](CCCC(C)(C)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 343 | 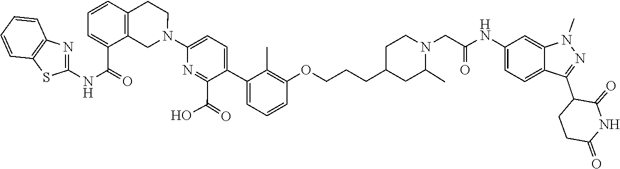 [SMILES: Cc1c(OCCCC2CCN(CC(=O)Nc3ccc4c(C5CCC(=O)NC5=O)nn(C)c4c3)C(C)C2)cccc1-c1ccc(N2CCc3cccc(C(=O)Nc4nc5ccccc5s4)c3C2)nc1C(=O)O] |
| 343a | 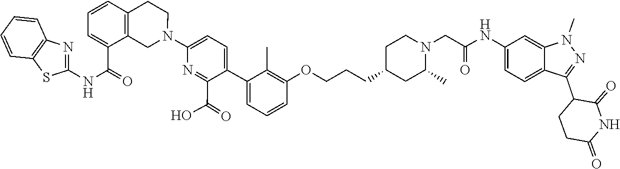 [SMILES: Cc1c(OCCC[C@@H]2CCN(CC(=O)Nc3ccc4c(C5CCC(=O)NC5=O)nn(C)c4c3)[C@H](C)C2)cccc1-c1ccc(N2CCc3cccc(C(=O)Nc4nc5ccccc5s4)c3C2)nc1C(=O)O] |
| 343b | 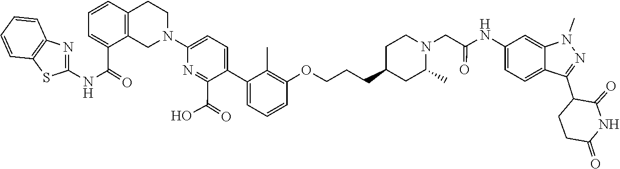 [SMILES: Cc1c(OCCC[C@H]2CCN(CC(=O)Nc3ccc4c(C5CCC(=O)NC5=O)nn(C)c4c3)[C@H](C)C2)cccc1-c1ccc(N2CCc3cccc(C(=O)Nc4nc5ccccc5s4)c3C2)nc1C(=O)O] |
| 343c | 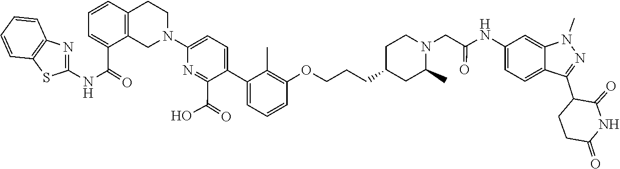 [SMILES: Cc1c(OCCC[C@@H]2CCN(CC(=O)Nc3ccc4c(C5CCC(=O)NC5=O)nn(C)c4c3)[C@@H](C)C2)cccc1-c1ccc(N2CCc3cccc(C(=O)Nc4nc5ccccc5s4)c3C2)nc1C(=O)O] |
| 343d | 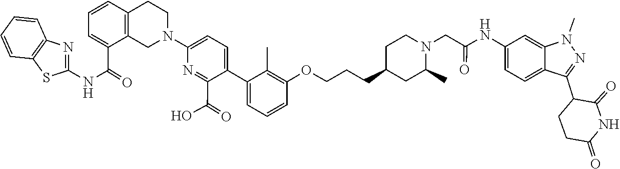 [SMILES: Cc1c(OCCC[C@H]2CCN(CC(=O)Nc3ccc4c(C5CCC(=O)NC5=O)nn(C)c4c3)[C@@H](C)C2)cccc1-c1ccc(N2CCc3cccc(C(=O)Nc4nc5ccccc5s4)c3C2)nc1C(=O)O] |
| 344 | 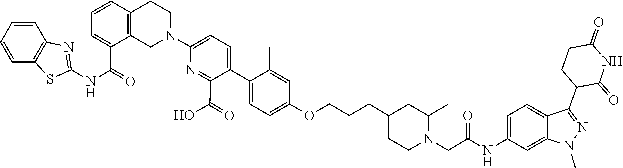 [SMILES: Cc1cc(OCCCC2CCN(CC(=O)Nc3ccc4c(C5CCC(=O)NC5=O)nn(C)c4c3)C(C)C2)ccc1-c1ccc(N2CCc3cccc(C(=O)Nc4nc5ccccc5s4)c3C2)nc1C(=O)O] |
| 344a | 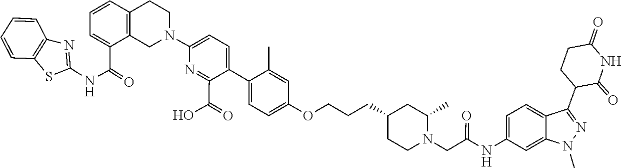 [SMILES: Cc1cc(OCCC[C@H]2CCN(CC(=O)Nc3ccc4c(C5CCC(=O)NC5=O)nn(C)c4c3)[C@@H](C)C2)ccc1-c1ccc(N2CCc3cccc(C(=O)Nc4nc5ccccc5s4)c3C2)nc1C(=O)O] |
| 344b | 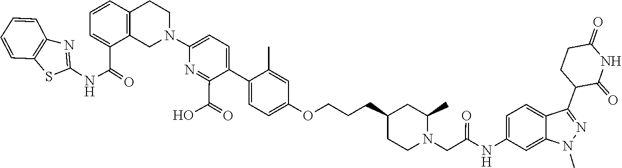 [SMILES: Cc1cc(OCCC[C@@H]2CCN(CC(=O)Nc3ccc4c(C5CCC(=O)NC5=O)nn(C)c4c3)[C@H](C)C2)ccc1-c1ccc(N2CCc3cccc(C(=O)Nc4nc5ccccc5s4)c3C2)nc1C(=O)O] |
| 344c | 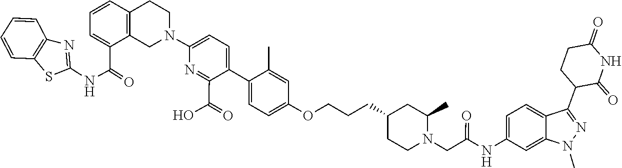 [SMILES: Cc1cc(OCCC[C@H]2CCN(CC(=O)Nc3ccc4c(C5CCC(=O)NC5=O)nn(C)c4c3)[C@H](C)C2)ccc1-c1ccc(N2CCc3cccc(C(=O)Nc4nc5ccccc5s4)c3C2)nc1C(=O)O] |
| 344d | 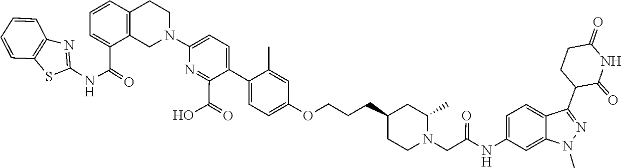 [SMILES: Cc1cc(OCCC[C@@H]2CCN(CC(=O)Nc3ccc4c(C5CCC(=O)NC5=O)nn(C)c4c3)[C@@H](C)C2)ccc1-c1ccc(N2CCc3cccc(C(=O)Nc4nc5ccccc5s4)c3C2)nc1C(=O)O] |
| 345 | 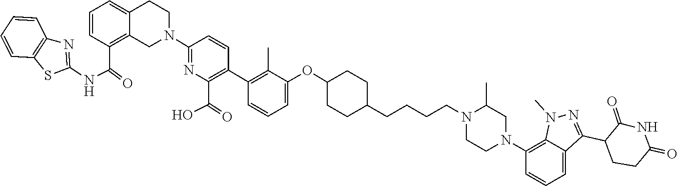 [SMILES: Cc1c(OC2CCC(CCCCN3CCN(c4cccc5c(C6CCC(=O)NC6=O)nn(C)c45)CC3C)CC2)cccc1-c1ccc(N2CCc3cccc(C(=O)Nc4nc5ccccc5s4)c3C2)nc1C(=O)O] |
| 345a | 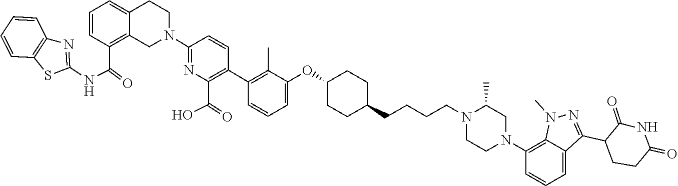 [SMILES: Cc1c(O[C@H]2CC[C@H](CCCCN3CCN(c4cccc5c(C6CCC(=O)NC6=O)nn(C)c45)C[C@H]3C)CC2)cccc1-c1ccc(N2CCc3cccc(C(=O)Nc4nc5ccccc5s4)c3C2)nc1C(=O)O] |
| 346 | 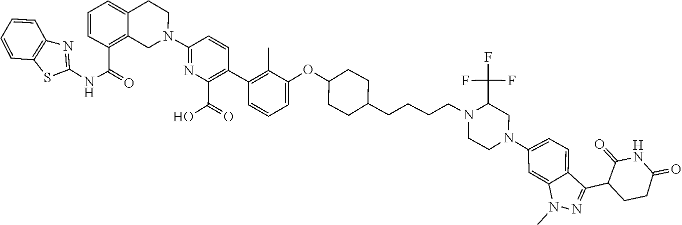 [SMILES: Cc1c(OC2CCC(CCCCN3CCN(c4ccc5c(C6CCC(=O)NC6=O)nn(C)c5c4)CC3C(F)(F)F)CC2)cccc1-c1ccc(N2CCc3cccc(C(=O)Nc4nc5ccccc5s4)c3C2)nc1C(=O)O] |
| 346a | 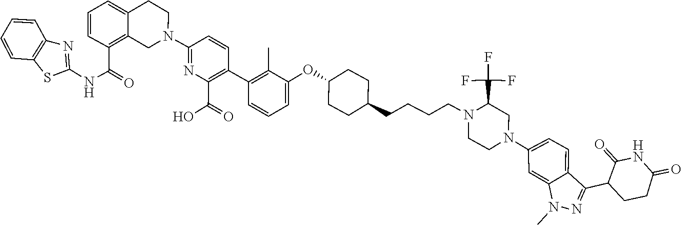 [SMILES: Cc1c(O[C@H]2CC[C@H](CCCCN3CCN(c4ccc5c(C6CCC(=O)NC6=O)nn(C)c5c4)C[C@@H]3C(F)(F)F)CC2)cccc1-c1ccc(N2CCc3cccc(C(=O)Nc4nc5ccccc5s4)c3C2)nc1C(=O)O] |
| 346b | 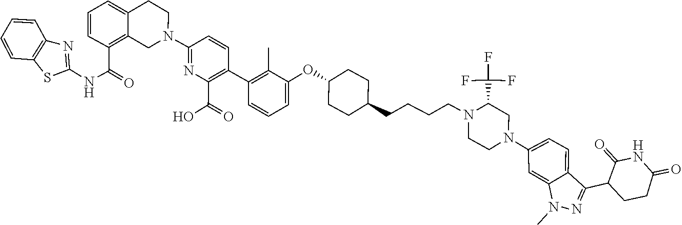 [SMILES: Cc1c(O[C@H]2CC[C@H](CCCCN3CCN(c4ccc5c(C6CCC(=O)NC6=O)nn(C)c5c4)C[C@H]3C(F)(F)F)CC2)cccc1-c1ccc(N2CCc3cccc(C(=O)Nc4nc5ccccc5s4)c3C2)nc1C(=O)O] |
| 347 | 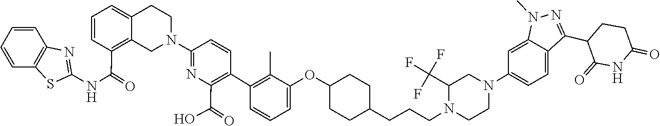 [SMILES: Cc1c(OC2CCC(CCCN3CCN(c4ccc5c(C6CCC(=O)NC6=O)nn(C)c5c4)CC3C(F)(F)F)CC2)cccc1-c1ccc(N2CCc3cccc(C(=O)Nc4nc5ccccc5s4)c3C2)nc1C(=O)O] |
| 347a | 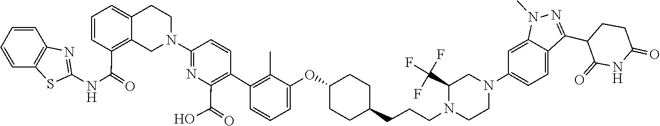 [SMILES: Cc1c(O[C@H]2CC[C@H](CCCN3CCN(c4ccc5c(C6CCC(=O)NC6=O)nn(C)c5c4)C[C@@H]3C(F)(F)F)CC2)cccc1-c1ccc(N2CCc3cccc(C(=O)Nc4nc5ccccc5s4)c3C2)nc1C(=O)O] |
| 347b | 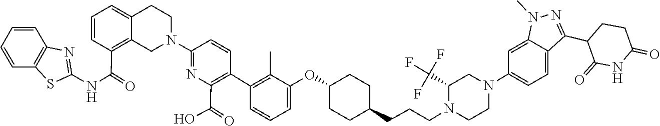 [SMILES: Cc1c(O[C@H]2CC[C@H](CCCN3CCN(c4ccc5c(C6CCC(=O)NC6=O)nn(C)c5c4)C[C@H]3C(F)(F)F)CC2)cccc1-c1ccc(N2CCc3cccc(C(=O)Nc4nc5ccccc5s4)c3C2)nc1C(=O)O] |
| 349 | 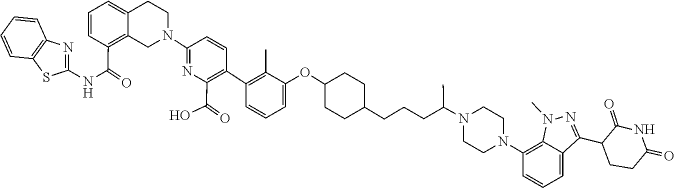 [SMILES: Cc1c(OC2CCC(CCCC(C)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 349a | 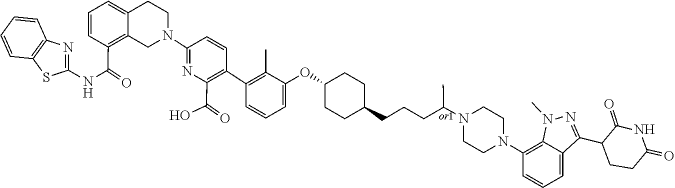 [SMILES: Cc1c(O[C@H]2CC[C@H](CCCC(C)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 349b | 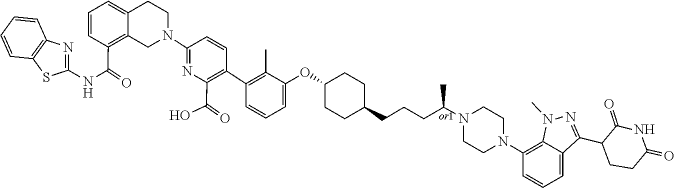 [SMILES: Cc1c(O[C@H]2CC[C@H](CCC[C@@H](C)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 350 | 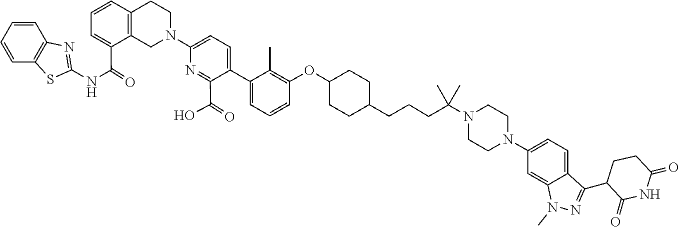 [SMILES: Cc1c(OC2CCC(CCCC(C)(C)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 350a | 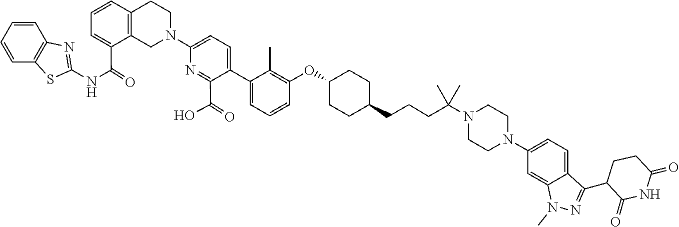 [SMILES: Cc1c(O[C@H]2CC[C@H](CCCC(C)(C)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 351 | 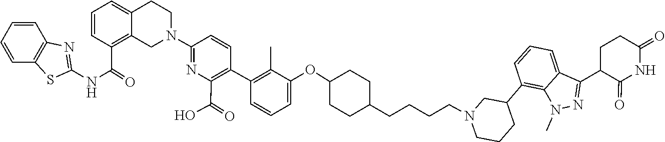 [SMILES: Cc1c(OC2CCC(CCCCN3CCCC(c4cccc5c(C6CCC(=O)NC6=O)nn(C)c45)C3)CC2)cccc1-c1ccc(N2CCc3cccc(C(=O)Nc4nc5ccccc5s4)c3C2)nc1C(=O)O] |
| 351a | 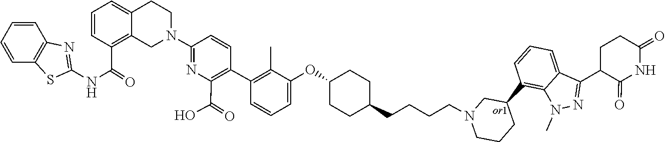 [SMILES: Cc1c(O[C@H]2CC[C@H](CCCCN3CCC[C@H](c4cccc5c(C6CCC(=O)NC6=O)nn(C)c45)C3)CC2)cccc1-c1ccc(N2CCc3cccc(C(=O)Nc4nc5ccccc5s4)c3C2)nc1C(=O)O] |
| 351b | 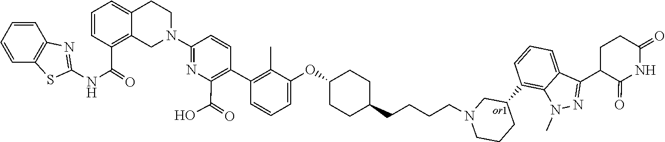 [SMILES: Cc1c(O[C@H]2CC[C@H](CCCCN3CCC[C@@H](c4cccc5c(C6CCC(=O)NC6=O)nn(C)c45)C3)CC2)cccc1-c1ccc(N2CCc3cccc(C(=O)Nc4nc5ccccc5s4)c3C2)nc1C(=O)O] |
| 352 | 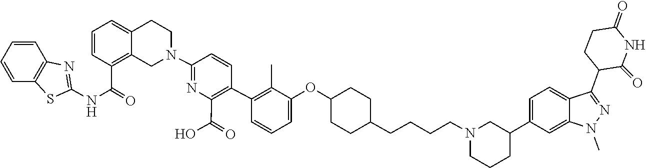 [SMILES: Cc1c(OC2CCC(CCCCN3CCCC(c4ccc5c(C6CCC(=O)NC6=O)nn(C)c5c4)C3)CC2)cccc1-c1ccc(N2CCc3cccc(C(=O)Nc4nc5ccccc5s4)c3C2)nc1C(=O)O] |
| 352a | 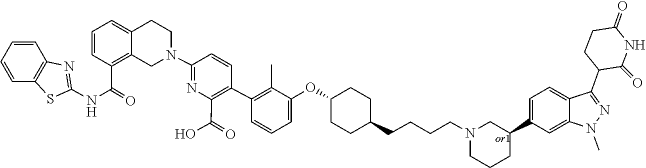 [SMILES: Cc1c(O[C@H]2CC[C@H](CCCCN3CCC[C@H](c4ccc5c(C6CCC(=O)NC6=O)nn(C)c5c4)C3)CC2)cccc1-c1ccc(N2CCc3cccc(C(=O)Nc4nc5ccccc5s4)c3C2)nc1C(=O)O] |
| 352b | 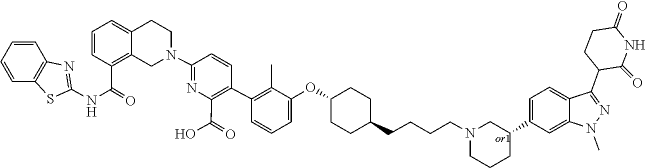 [SMILES: Cc1c(O[C@H]2CC[C@H](CCCCN3CCC[C@@H](c4ccc5c(C6CCC(=O)NC6=O)nn(C)c5c4)C3)CC2)cccc1-c1ccc(N2CCc3cccc(C(=O)Nc4nc5ccccc5s4)c3C2)nc1C(=O)O] |
| 353 | 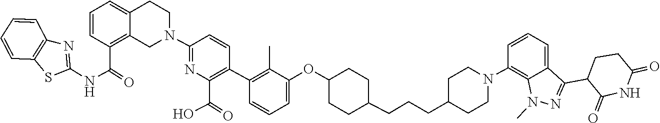 [SMILES: Cc1c(OC2CCC(CCCC3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 353a | 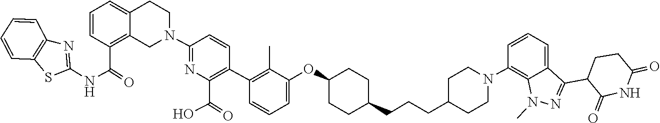 [SMILES: Cc1c(O[C@H]2CC[C@@H](CCCC3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 353b | 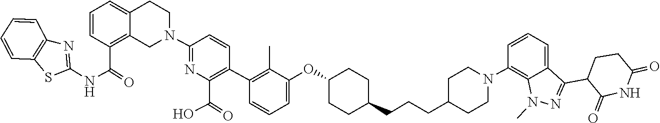 [SMILES: Cc1c(O[C@H]2CC[C@H](CCCC3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 354 | 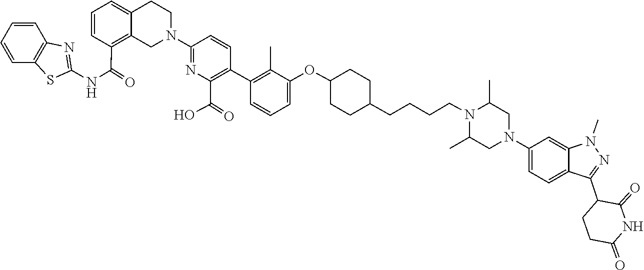 [SMILES: Cc1c(OC2CCC(CCCCN3C(C)CN(c4ccc5c(C6CCC(=O)NC6=O)nn(C)c5c4)CC3C)CC2)cccc1-c1ccc(N2CCc3cccc(C(=O)Nc4nc5ccccc5s4)c3C2)nc1C(=O)O] |
| 354a | 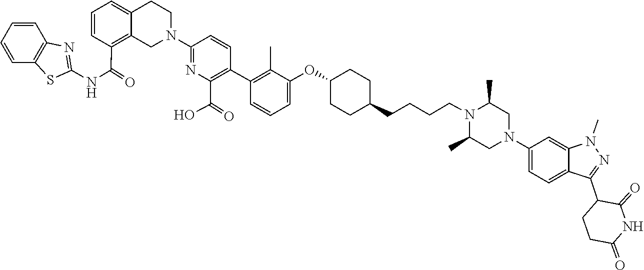 [SMILES: Cc1c(O[C@H]2CC[C@H](CCCCN3[C@H](C)CN(c4ccc5c(C6CCC(=O)NC6=O)nn(C)c5c4)C[C@@H]3C)CC2)cccc1-c1ccc(N2CCc3cccc(C(=O)Nc4nc5ccccc5s4)c3C2)nc1C(=O)O] |
| 354b | 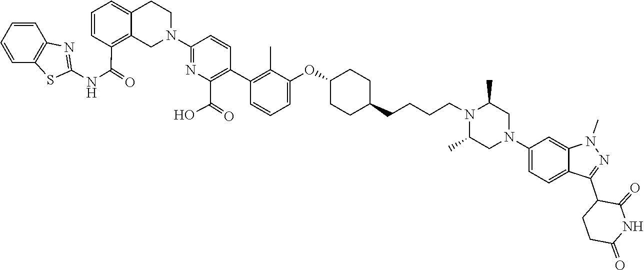 [SMILES: Cc1c(O[C@H]2CC[C@H](CCCCN3[C@@H](C)CN(c4ccc5c(C6CCC(=O)NC6=O)nn(C)c5c4)C[C@@H]3C)CC2)cccc1-c1ccc(N2CCc3cccc(C(=O)Nc4nc5ccccc5s4)c3C2)nc1C(=O)O] |
| 355 | 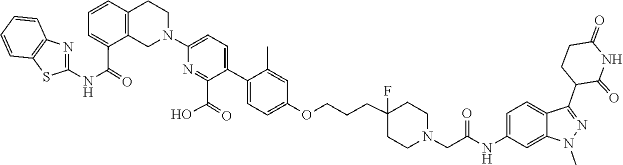 [SMILES: Cc1cc(OCCCC2(F)CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 356 | 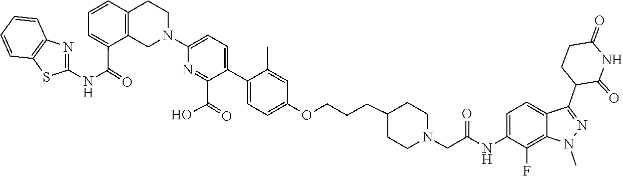 [SMILES: Cc1cc(OCCCC2CCN(CC(=O)Nc3ccc4c(C5CCC(=O)NC5=O)nn(C)c4c3F)CC2)ccc1-c1ccc(N2CCc3cccc(C(=O)Nc4nc5ccccc5s4)c3C2)nc1C(=O)O] |
| 357 | 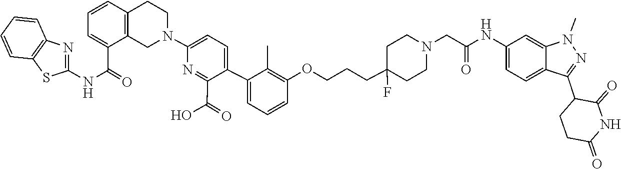 [SMILES: Cc1c(OCCCC2(F)CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |

Note:
for compounds in Table C1, when a stereogenic center is denoted with “or 1” in a structural formula, the stereogenic center has been resolved but the configuration at the stereogenic center has not been determined. For example, the structure 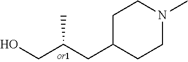
represents (R)-2-methyl-3-(1-methylpiperidin-4-yl)propan-1-ol or (S)-2-methyl-3-(1-methylpiperidin-4-yl)propan-1-ol. For example, the structure 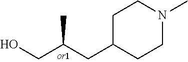
represents (R)-2-methyl-3-(1-methylpiperidin-4-yl)propan-1-ol or (S)-2-methyl-3-(1-methylpiperidin-4-yl)propan-1-ol.

Exemplary compounds of Formula (I) or (II) also include those depicted in Table C1 of U.S. Provisional Application Ser. No. 63/457,997, filed Apr. 7, 2023; Table C1 of U.S. Provisional Application Ser. No. 63/454,545, filed Mar. 24, 2023; Table C1 of U.S. Provisional Application Ser. No. 63/449,756, filed Mar. 3, 2023; Table C1 of U.S. Provisional Application Ser. No. 63/429,862, filed Dec. 2, 2022; Table C1 of U.S. Provisional Application Ser. No. 63/398,783, filed Aug. 17, 2022; and Table C1 of U.S. Provisional Application Ser. No. 63/339,253, filed May 6, 2022; or pharmaceutically acceptable salts thereof, wherein each Table C1 is incorporated herein by reference in its entirety.

Exemplary compounds of Formula (I-A) (e.g., Formula (I-A-1), (I-A-2), or (I-A-3)) include compounds 101, 102, 103, 104, 105, 106, 107, 116, 116a, 117, 117a, 120, 121, 122, 124, 124a, 125, 125a, 126, 127, 128, 129, 130, 131, 131a, 132, 134, 156, 158, 159, 159a, 160, 160a, 160b, 162, 162a, 162b, 164, 164a, 164b, 166, 167, 167a, 167b, 169, 169a, 169b, 170, 173, 173a, 173b, 177, 177a, 180, 180a, 180b, 181, 186, 186a, 186b, 187, 187a, 187b, 193, 193a, 193b, 196, 197, 197a, 197b, 202, 202a, 202b, 203, 207, 207a, 207b, 209, 209a, 210, 210a, 210b, 213, 216, 216a, 216b, 218, 218a, 218b, 219, 219a, 219b, 226, 229, 231, 231a, 231b, 240, 240a, 240b, 243, 245, 245a, 245b, 246, 246a, 246b, 262, 262a, 262b, 263, 263a, 263b, 267, 268, 268a, 268b, 272, 272a, 272b, 277, 280, 286, 289, 289a, 289b, 292, 295, 302, 309, 313, 317, 343, 343a, 343b, 343c, 343d, and 357, as depicted in Table C1, or pharmaceutically acceptable salts thereof.

Exemplary compounds of Formula (I-B) (e.g., Formula (I-B-1), (I-B-2), or (I-B-3)) include compounds 119, 139, 184, 236, 242, 264, 265, 266, 266a, 266b, 271, 271a, 276, 276a, 276b, 294, 344, 344a, 344b, 344c, 344d, 355, and 356, as depicted in Table C1, or pharmaceutically acceptable salts thereof.

Exemplary compounds of Formula (I-C) include compounds 123, 133, 136, 165, and 172 as depicted in Table C1, or pharmaceutically acceptable salts thereof.

Exemplary compounds of Formula (I-E) (e.g., Formula (I-E-1), (I-E-2), or (I-E-3)) include compounds 147, 147a, 147b, 148, 148a, 148b, 149, 150, 161, 161a, 163, 168, 168a, 175, 188, 195, 200, 201, 204, 204a, 208, 211, 211a, 212, 212a, 215, 215a, 220, 225, 230, 232, 233, 234, 235, 241, 273, 273a, 273b, 274, 274a, 274b, 275, 275a, 275b, 278, 278a, 278b, 281, 281a, 281b, 282, 282a, 282b, 283, 283a, 283b, 284, 284a, 284b, 293, 293a, 296, 296a, 297, 297a, 297b, 300, 300a, 300b, 301, 301a, 301b, 307, 307a, 307b, 308, 308a, 308b, 310, 310a, 316, 316a, 318, 318a, 319, 319a, 322, 322a, 323, 323a, 324, 324a, 325, 325a, 328, 328a, 333, 333a, 334, 334a, 335, 335a, 336, 336a, 339, 339a, 339b, 340, 340a, 341, 341a, 342, 342a, 345, 345a, 346, 346a, 346b, 347, 347a, 347b, 349, 349a, 349b, 350, 350a, 354, 354a, and 354b as depicted in Table C1, or pharmaceutically acceptable salts thereof.

Exemplary compounds of Formula (I-Ea) (e.g., Formula (I-Ea-1), (I-Ea-2), or (I-Ea-3)) include compounds 189, 192, 198, 221, 223, 228, 247, 257, 258, 261, 279, 285, 287, 288, 306, 311, 311a, 311b, 312, 353, 353a, and 353b as depicted in Table C1, or pharmaceutically acceptable salts thereof.

Exemplary compounds of Formula (I-Eb) (e.g., Formula (I-Eb-1), (I-Eb-2), or (I-Eb-3)) include compounds 248, 249, 255, 256, 269, 269a, 270, 270a, 298, 298a, 299, 299a, 320, 320a, 320b, 321, 321a, 321b, 331, 331a, 331b, 332, 332a, 332b, 351, 351a, 351b, 352, 352a, and 352b as depicted in Table C1, or pharmaceutically acceptable salts thereof.

Exemplary compounds of Formula (I-F) (e.g., Formula (I-F-1), (I-F-2), or (I-F-3)) include compounds 138, 138a, 138b, 140, 141, 142, 143, 144, 145, 146, 146a, 146b, 151, 171, 171a, 176, 176a, 178, 179, 179a, 182, 182a, 185, 185a, 191, 214, 214a, 227, 227a, 239, 239a, and 244 as depicted in Table C1, or pharmaceutically acceptable salts thereof.

Exemplary compounds of Formula (I-G) include compounds 190, 199, 250, 250a, 254, and 254a as depicted in Table C1, or pharmaceutically acceptable salts thereof.

Exemplary compounds of Formula (I-H) (e.g., Formula (I-H-1), (I-H-2), or (I-H-3)) include compounds 152, 153, 154, 154a, 183, 183a, 251, 251a, 252, and 252a as depicted in Table C1, or pharmaceutically acceptable salts thereof.

In some embodiments, the compounds of Formula (I) or (II) are selected from the group consisting of the compounds in Table C2, or pharmaceutically acceptable salts thereof.

TABLE C2

| No. | Compound Name |
|---|---|
| 101 | 6-[8-(1,3-benzothiazol-2-ylcarbamoyl)-3,4-dihydro-1H-isoquinolin-2-yl]-3-[3-[2-[1- |
| | [2-[[3-(2,6-dioxo-3-piperidyl)-1-methyl-indazol-7-yl]amino]-2-oxo-ethyl]-4- |
| | piperidyl]ethoxy]-2-methyl-phenyl]pyridine-2-carboxylic acid |
| 102 | 6-[8-(1,3-benzothiazol-2-ylcarbamoyl)-3,4-dihydro-1H-isoquinolin-2-yl]-3-[3-[4-[3- |
| | [4-(2,6-dioxo-3-piperidyl)anilino]-3-oxo-propyl]phenoxy]-2-methyl-phenyl]pyridine- |
| | 2-carboxylic acid |
| 103 | 6-[8-(1,3-benzothiazol-2-ylcarbamoyl)-3,4-dihydro-1H-isoquinolin-2-yl]-3-[3-[2-[4- |
| | [2-[[3-(2,6-dioxo-3-piperidyl)-1-methyl-indazol-6-yl]amino]-2-oxo-ethyl]piperazin-1- |
| | yl]ethoxy]-2-methyl-phenyl]pyridine-2-carboxylic acid |
| 104 | 6-[8-(1,3-benzothiazol-2-ylcarbamoyl)-3,4-dihydro-1H-isoquinolin-2-yl]-3-[3-[3-[4- |
| | [2-[[3-(2,6-dioxo-3-piperidyl)-1-methyl-indazol-6-yl]amino]-2-oxo-ethyl]piperazin-1- |
| | yl]propoxy]-2-methyl-phenyl]pyridine-2-carboxylic acid |
| 105 | 6-[8-(1,3-benzothiazol-2-ylcarbamoyl)-3,4-dihydro-1H-isoquinolin-2-yl]-3-[3-[3-[4- |
| | [2-[[3-(2,6-dioxo-3-piperidyl)-1-methyl-indazol-7-yl]amino]-2-oxo-ethyl]piperazin-1- |
| | yl]propoxy]-2-methyl-phenyl]pyridine-2-carboxylic acid |
| 106 | 6-[8-(1,3-benzothiazol-2-ylcarbamoyl)-3,4-dihydro-1H-isoquinolin-2-yl]-3-[3-[2-[1- |
| | [2-[[3-(2,6-dioxo-3-piperidyl)-1-methyl-indazol-6-yl]amino]-2-oxo-ethyl]-4- |
| | piperidyl]ethoxy]-2-methyl-phenyl]pyridine-2-carboxylic acid |
| 107 | 6-[8-(1,3-benzothiazol-2-ylcarbamoyl)-3,4-dihydro-1H-isoquinolin-2-yl]-3-[3-[4-[3- |
| | [[3-(2,6-dioxo-3-piperidyl)-1-methyl-indazol-6-yl]amino]-3-oxo-propyl]phenoxy]-2- |
| | methyl-phenyl]pyridine-2-carboxylic acid |
| 108 | 2-(5-(1-(((3r,5r,7r)-adamantan-1-yl)methyl)-5-methyl-1H-pyrazol-4-yl)-6-((6-((3-(2,6- |
| | dioxopiperidin-3-yl)-1-methyl-1H-indazol-7-yl)amino)-6- |
| | oxohexyl)carbamoyl)pyridin-2-yl)-N-(benzo[d]thiazol-2-yl)-1,2,3,4- |
| | tetrahydroisoquinoline-8-carboxamide |
| 109 | 2-(5-(1-((3R,5R,7R)-adamantan-1-ylmethyl)-5-methyl-1H-pyrazol-4-yl)-6-((10- |
| | ((3-(2,6-dioxopiperidin-3-yl)-1-methyl-1H-indazol-7-yl) amino)-10-oxodecyl) |
| | carbamoyl) pyridin-2-yl)-N-(benzo[d]thiazol-2-yl)-1,2,3,4-tetrahydroisoquinoline- |
| | 8-carboxamide |
| 110 | 2-(5-(1-((3r,5r,7r)-adamantan-1-ylmethyl)-5-methyl-1H-pyrazol-4-yl)-6-((12-((3-(2,6- |
| | dioxopiperidin-3-yl)-1-methyl-1H-indazol-7-yl)amino)-12- |
| | oxododecyl)carbamoyl)pyridin-2-yl)-N-(benzo[d]thiazol-2-yl)-1,2,3,4- |
| | tetrahydroisoquinoline-8-carboxamide |
| 111 | 2-(5-(1-(((3r,5r,7r)-adamantan-1-yl)methyl)-5-methyl-1H-pyrazol-4-yl)-6-((6-((3-(2,6- |
| | dioxopiperidin-3-yl)-1-methyl-1H-indazol-6-yl)amino)-6- |
| | oxohexyl)carbamoyl)pyridin-2-yl)-N-(benzo[d]thiazol-2-yl)-1,2,3,4- |
| | tetrahydroisoquinoline-8-carboxamide |
| 112 | 2-[5-[1-(1-adamantylmethyl)-5-methyl-pyrazol-4-yl]-6-[[10-[[3-(2,6-dioxo-3- |
| | piperidyl)-1-methyl-indazol-6-yl]amino]-10-oxo-decyl]carbamoyl]-2-pyridyl]-N-(1,3- |
| | benzothiazol-2-yl)-3,4-dihydro-1H-isoquinoline-8-carboxamide |
| 113 | 2-(5-(1-((3r,5r,7r)-adamantan-1-ylmethyl)-5-methyl-1H-pyrazol-4-yl)-6-((12-((3-(2,6- |
| | dioxopiperidin-3-yl)-1-methyl-1H-indazol-6-yl)amino)-12- |
| | oxododecyl)carbamoyl)pyridin-2-yl)-N-(benzo[d]thiazol-2-yl)-1,2,3,4- |
| | tetrahydroisoquinoline-8-carboxamide |
| 114 | 2-[5-[1-(1-adamantylmethyl)-5-methyl-pyrazol-4-yl]-6-[[8-[[3-(2,6-dioxo-3- |
| | piperidyl)-1-methyl-indazol-7-yl]amino]-8-oxo-octyl]carbamoyl]-2-pyridyl]-N-(1,3- |
| | benzothiazol-2-yl)-3,4-dihydro-1H-isoquinoline-8-carboxamide |
| 115 | 2-(5-(1-((1R,3S)-adamantan-1-ylmethyl)-5-methyl-1H-pyrazol-4-yl)-6-((2-((3-(2,6- |
| | dioxopiperidin-3-yl)-1-methyl-1H-indazol-6-yl)amino)-2-oxoethyl)carbamoyl)pyridin- |
| | 2-yl)-N-(benzo[d]thiazol-2-yl)-1,2,3,4-tetrahydroisoquinoline-8-carboxamide |
| 116 | 6-[8-(1,3-benzothiazol-2-ylcarbamoyl)-3,4-dihydro-1H-isoquinolin-2-yl]-3-[3-[3-[4- |
| | [2-[[3-(2,6-dioxo-3-piperidyl)-1-methyl-indazol-6-yl]amino]-2-oxo-ethyl]-3,5- |
| | dimethyl-piperazin-1-yl]propoxy]-2-methyl-phenyl]pyridine-2-carboxylic acid |
| 116a | 6-[8-(1,3-benzothiazol-2-ylcarbamoyl)-3,4-dihydro-1H-isoquinolin-2-yl]-3-[3-[3- |
| | [(3S,5R)-4-[2-[[3-(2,6-dioxo-3-piperidyl)-1-methyl-indazol-6-yl]amino]-2-oxo-ethyl]- |
| | 3,5-dimethyl-piperazin-1-yl]propoxy]-2-methyl-phenyl]pyridine-2-carboxylic acid |
| 117 | 6-[8-(1,3-benzothiazol-2-ylcarbamoyl)-3,4-dihydro-1H-isoquinolin-2-yl]-3-[3-[3-[4- |
| | [2-[[3-(2,6-dioxo-3-piperidyl)-1-methyl-indazol-7-yl]amino]-2-oxo-ethyl]-3,5- |
| | dimethyl-piperazin-1-yl]propoxy]-2-methyl-phenyl]pyridine-2-carboxylic acid |
| 117a | 6-[8-(1,3-benzothiazol-2-ylcarbamoyl)-3,4-dihydro-1H-isoquinolin-2-yl]-3-[3-[3- |
| | [(3S,5R)-4-[2-[[3-(2,6-dioxo-3-piperidyl)-1-methyl-indazol-7-yl]amino]-2-oxo-ethyl]- |
| | 3,5-dimethyl-piperazin-1-yl]propoxy]-2-methyl-phenyl]pyridine-2-carboxylic acid |
| 118 | 6-[8-(1,3-benzothiazol-2-ylcarbamoyl)-3,4-dihydro-1H-isoquinolin-2-yl]-3-[3-[2-[4- |
| | [2-[[3-(2,6-dioxo-3-piperidyl)-1-methyl-indazol-7-yl]amino]-2-oxo- |
| | ethyl]phenoxy]ethoxy]-2-methyl-phenyl]pyridine-2-carboxylic acid |
| 119 | 6-[8-(1,3-benzothiazol-2-ylcarbamoyl)-3,4-dihydro-1H-isoquinolin-2-yl]-3-[4-[3-[4- |
| | [2-[[3-(2,6-dioxo-3-piperidyl)-1-methyl-indazol-6-yl]amino]-2-oxo-ethyl]piperazin-1- |
| | yl]propoxy]-2-methyl-phenyl]pyridine-2-carboxylic acid |
| 120 | 6-[8-(1,3-benzothiazol-2-ylcarbamoyl)-3,4-dihydro-1H-isoquinolin-2-yl]-3-[3-[3-[4- |
| | [2-[[3-(2,6-dioxo-3-piperidyl)-1-methyl-indazol-6-yl]amino]-2-oxo-ethyl]-1- |
| | piperidyl]propoxy]-2-methyl-phenyl]pyridine-2-carboxylic acid |
| 121 | 6-(8-(Benzo[d]thiazol-2-ylcarbamoyl)-3,4-dihydroisoquinolin-2(1H)-yl)-3-(3-(3-(1-(2- |
| | ((3-(2,6-dioxopiperidin-3-yl)-1-methyl-1H-indazol-6-yl)amino)-2-oxoethyl)piperidin- |
| | 4-yl)propoxy)-2-methylphenyl)picolinic acid |
| 122 | 6-[8-(1,3-benzothiazol-2-ylcarbamoyl)-3,4-dihydro-1H-isoquinolin-2-yl]-3-[3-[3-[4- |
| | [2-[[3-(2,6-dioxo-3-piperidyl)-1-methyl-indazol-7-yl]amino]-2-oxo-ethyl]-1- |
| | piperidyl]propoxy]-2-methyl-phenyl]pyridine-2-carboxylic acid |
| 123 | 6-[8-(1,3-benzothiazol-2-ylcarbamoyl)-3,4-dihydro-1H-isoquinolin-2-yl]-3-[3-[4-[3- |
| | [[3-(2,6-dioxo-3-piperidyl)-1-methyl-indazol-6-yl]amino]propyl]phenoxy]-2-methyl- |
| | phenyl]pyridine-2-carboxylic acid |
| 124 | 6-[8-(1,3-benzothiazol-2-ylcarbamoyl)-3,4-dihydro-1H-isoquinolin-2-yl]-3-[3-[4-[3- |
| | [[3-(2,6-dioxo-3-piperidyl)-1-methyl-indazol-7-yl]amino]-3-oxo-propyl]cyclohexoxy]- |
| | 2-methyl-phenyl]pyridine-2-carboxylic acid |
| 124a | 6-(8-(benzo[d]thiazol-2-ylcarbamoyl)-3,4-dihydroisoquinolin-2(1H)-yl)-3-(3-(((1r,4r)- |
| | 4-(3-((3-(2,6-dioxopiperidin-3-yl)-1-methyl-1H-indazol-7-yl)amino)-3- |
| | oxopropyl)cyclohexyl)oxy)-2-methylphenyl)picolinic acid |
| 125 | 6-[8-(1,3-benzothiazol-2-ylcarbamoyl)-3,4-dihydro-1H-isoquinolin-2-yl]-3-[3-[4-[3- |
| | [[3-(2,6-dioxo-3-piperidyl)-1-methyl-indazol-6-yl]amino]-3-oxo-propyl]cyclohexoxy]- |
| | 2-methyl-phenyl]pyridine-2-carboxylic acid |
| 125a | 6-(8-(benzo[d]thiazol-2-ylcarbamoyl)-3,4-dihydroisoquinolin-2(1H)-yl)-3-(3-(((1r,4r)- |
| | 4-(3-((3-(2,6-dioxopiperidin-3-yl)-1-methyl-1H-indazol-6-yl)amino)-3- |
| | oxopropyl)cyclohexyl)oxy)-2-methylphenyl)picolinic acid |
| 126 | 6-(8-(benzo[d]thiazol-2-ylcarbamoyl)-3,4-dihydroisoquinolin-2(1H)-yl)-3-(3-(2-(4-(2- |
| | ((3-(2,6-dioxopiperidin-3-yl)-1-methyl-1H-indazol-6-yl)amino)-2-oxoethyl)piperazin- |
| | 1-yl)ethoxy)-2-(trifluoromethyl)phenyl)picolinic acid |
| 127 | 6-(8-(benzo[d]thiazol-2-ylcarbamoyl)-3,4-dihydroisoquinolin-2(1H)-yl)-3-(3-(3- |
| | ((1R,5S)-8-(2-((3-(2,6-dioxopiperidin-3-yl)-1-methyl-1H-indazol-6-yl)amino)-2- |
| | oxoethyl)-3,8-diazabicyclo[3.2.1]octan-3-yl)propoxy)-2-methylphenyl)picolinic acid |
| 128 | 6-(8-(benzo[d]thiazol-2-ylcarbamoyl)-3,4-dihydroisoquinolin-2(1H)-yl)-3-(3-(2-(4- |
| | ((3-(2,6-dioxopiperidin-3-yl)-1-methyl-1H-indazol-7-yl)amino)-4-oxobutyl)phenoxy)- |
| | 2-methylphenyl)picolinic acid |
| 129 | 6-(8-(benzo[d]thiazol-2-ylcarbamoyl)-3,4-dihydroisoquinolin-2(1H)-yl)-3-(3-(2-(4- |
| | ((3-(2,6-dioxopiperidin-3-yl)-1-methyl-1H-indazol-6-yl)amino)-4-oxobutyl)phenoxy)- |
| | 2-methylphenyl)picolinic acid |
| 130 | 6-[8-(1,3-Benzothiazol-2-ylcarbamoyl)-3,4-dihydro-1H-isoquinolin-2-yl]-3-[3-[4-[4- |
| | [2-[[3-(2,6-dioxo-3-piperidyl)-1-methyl-indazol-6-yl]amino]-2-oxo-ethyl]piperazin-1- |
| | yl]butyl]-2-methyl-phenyl]pyridine-2-carboxylic acid |
| 131 | 6-[8-(1,3-benzothiazol-2-ylcarbamoyl)-3,4-dihydro-1H-isoquinolin-2-yl]-3-[3-[3-[4- |
| | [2-[[3-(2,6-dioxo-3-piperidyl)-1-methyl-indazol-6-yl]amino]-2-oxo-ethyl]-2,6- |
| | dimethyl-piperazin-1-yl]propoxy]-2-methyl-phenyl]pyridine-2-carboxylic acid |
| 131a | 6-[8-(1,3-benzothiazol-2-ylcarbamoyl)-3,4-dihydro-1H-isoquinolin-2-yl]-3-[3-[3- |
| | [(2R,6S)-4-[2-[[3-(2,6-dioxo-3-piperidyl)-1-methyl-indazol-6-yl]amino]-2-oxo-ethyl]- |
| | 2,6-dimethyl-piperazin-1-yl]propoxy]-2-methyl-phenyl]pyridine-2-carboxylic acid |
| 132 | 6-(8-(benzo[d]thiazol-2-ylcarbamoyl)-3,4-dihydroisoquinolin-2(1H)-yl)-3-(3-(4-(4- |
| | ((3-(2,6-dioxopiperidin-3-yl)-1-methyl-1H-indazol-6-yl)amino)-4-oxobutyl)phenoxy)- |
| | 2-methylphenyl)picolinic acid |
| 133 | 6-(8-(Benzo[d]thiazol-2-ylcarbamoyl)-3,4-dihydroisoquinolin-2(1H)-yl)-3-(3-(3-(4-(3- |
| | ((3-(2,6-dioxopiperidin-3-yl)-1-methyl-1H-indazol-6-yl)oxy)propyl)piperazin-1- |
| | yl)propoxy)-2-methylphenyl)picolinic acid |
| 134 | 6-(8-(benzo[d]thiazol-2-ylcarbamoyl)-3,4-dihydroisoquinolin-2(1H)-yl)-3-(3-(2-(3- |
| | ((4-(2,6-dioxopiperidin-3-yl)phenyl)amino)-3-oxopropyl)phenoxy)-2- |
| | methylphenyl)picolinic acid |
| 135 | N-(1,3-benzothiazol-2-yl)-2-[5-(1-benzyl-5-methyl-pyrazol-4-yl)-6-[2-[[3-(2,6-dioxo- |
| | 3-piperidyl)-1-methyl-indazol-6-yl]amino]-2-oxo-ethyl]carbamoyl]-2-pyridyl]-3,4- |
| | dihydro-1H-isoquinoline-8-carboxamide |
| 136 | 6-(8-(benzo[d]thiazol-2-ylcarbamoyl)-3,4-dihydroisoquinolin-2(1H)-yl)-3-(3-(3-(4-(2- |
| | ((3-(2,6-dioxopiperidin-3-yl)-1-methyl-1H-indazol-6-yl)oxy)ethyl)piperazin-1- |
| | yl)propoxy)-2-methylphenyl)picolinic acid |
| 137 | 6-(8-(benzo[d]thiazol-2-ylcarbamoyl)-3,4-dihydroisoquinolin-2(1H)-yl)-3-(3-((7-(4- |
| | (3-(2,6-dioxopiperidin-3-yl)-1-methyl-1H-indazol-6-yl)piperazin-1-yl)heptyl)oxy)-2- |
| | methylphenyl)picolinic acid |
| 138 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-6-yl]piperazin-1-yl]-3-oxo- |
| | propyl]cyclohexoxy]-2-methyl-phenyl]pyridine-2-carboxylic acid |
| 138a | 6-(8-(benzo[d]thiazol-2-ylcarbamoyl)-3,4-dihydroisoquinolin-2(1H)-yl)-3-(3-(((1r,4r)- |
| | 4-(3-(4-(3-(2,6-dioxopiperidin-3-yl)-1-methyl-1H-indazol-6-yl)piperazin-1-yl)-3- |
| | oxopropyl)cyclohexyl)oxy)-2-methylphenyl)picolinic acid |
| 138b | 6-(8-(benzo[d]thiazol-2-ylcarbamoyl)-3,4-dihydroisoquinolin-2(1H)-yl)-3-(3-(((1s,4s)- |
| | 4-(3-(4-(3-(2,6-dioxopiperidin-3-yl)-1-methyl-1H-indazol-6-yl)piperazin-1-yl)-3- |
| | oxopropyl)cyclohexyl)oxy)-2-methylphenyl)picolinic acid |
| 139 | 6-(8-(benzo[d]thiazol-2-ylcarbamoyl)-3,4-dihydroisoquinolin-2(1H)-yl)-3-(4-(4-(3- |
| | ((3-(2,6-dioxopiperidin-3-yl)-1-methyl-1H-indazol-6-yl)amino)-3- |
| | oxopropyl)phenoxy)-2-methylphenyl)picolinic acid |
| 140 | 6-(8-(Benzo[d]thiazol-2-ylcarbamoyl)-3,4-dihydroisoquinolin-2(1H)-yl)-3-(3-(3-(4-(2- |
| | (4-(3-(2,6-dioxopiperidin-3-yl)-1-methyl-1H-indazol-6-yl)piperazin-1-yl)-2- |
| | oxoethyl)piperazin-1-yl)propoxy)-2-methylphenyl)picolinic acid |
| 141 | 6-(8-(benzo[d]thiazol-2-ylcarbamoyl)-3,4-dihydroisoquinolin-2(1H)-yl)-3-(3-(2-(4-(2- |
| | (4-(3-(2,6-dioxopiperidin-3-yl)-1-methyl-1H-indazol-6-yl)piperazin-1-yl)-2- |
| | oxoethyl)piperazin-1-yl)ethoxy)-2-methylphenyl)picolinic acid |
| 142 | 6-(8-(benzo[d]thiazol-2-ylcarbamoyl)-3,4-dihydroisoquinolin-2(1H)-yl)-3-(3-(3-(1-(2- |
| | (4-(3-(2,6-dioxopiperidin-3-yl)-1-methyl-1H-indazol-6-yl)piperazin-1-yl)-2- |
| | oxoethyl)piperidin-4-yl)propoxy)-2-methylphenyl)picolinic acid |
| 143 | 6-(8-(benzo[d]thiazol-2-ylcarbamoyl)-3,4-dihydroisoquinolin-2(1H)-yl)-3-(3-(2-(1-(2- |
| | (4-(3-(2,6-dioxopiperidin-3-yl)-1-methyl-1H-indazol-6-yl)piperazin-1-yl)-2- |
| | oxoethyl)piperidin-4-yl)ethoxy)-2-methylphenyl)picolinic acid |
| 144 | 6-[8-(1,3-benzothiazol-2-ylcarbamoyl)-3,4-dihydro-1H-isoquinolin-2-yl]-3-[3-[3-[1- |
| | [2-[4-[3-(2,6-dioxo-3-piperidyl)-1-methyl-indazol-7-yl]piperazin-1-yl]-2-oxo-ethyl]-4- |
| | piperidyl]propoxy]-2-methyl-phenyl]pyridine-2-carboxylic acid |
| 145 | 6-(8-(benzo[d]thiazol-2-ylcarbamoyl)-3,4-dihydroisoquinolin-2(1H)-yl)-3-(3-(2-(1-(2- |
| | (4-(3-(2,6-dioxopiperidin-3-yl)-1-methyl-1H-indazol-7-yl)piperazin-1-yl)-2- |
| | oxoethyl)piperidin-4-yl)ethoxy)-2-methylphenyl)picolinic acid |
| 146 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-7-yl]piperazin-1-yl]-3-oxo- |
| | propyl]cyclohexoxy]-2-methyl-phenyl]pyridine-2-carboxylic acid |
| 146a | 6-(8-(benzo[d]thiazol-2-ylcarbamoyl)-3,4-dihydroisoquinolin-2(1H)-yl)-3-(3-(((1r,4r)- |
| | 4-(3-(4-(3-(2,6-dioxopiperidin-3-yl)-1-methyl-1H-indazol-7-yl)piperazin-1-yl)-3- |
| | oxopropyl)cyclohexyl)oxy)-2-methylphenyl)picolinic acid |
| 146b | 6-(8-(benzo[d]thiazol-2-ylcarbamoyl)-3,4-dihydroisoquinolin-2(1H)-yl)-3-(3-(((1s,4s)- |
| | 4-(3-(4-(3-(2,6-dioxopiperidin-3-yl)-1-methyl-1H-indazol-7-yl)piperazin-1-yl)-3- |
| | oxopropyl)cyclohexyl)oxy)-2-methylphenyl)picolinic acid |
| 147 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-7-yl]piperazin-1- |
| | yl]propyl]cyclohexoxy]-2-methyl-phenyl]pyridine-2-carboxylic acid |
| 147a | 6-(8-(benzo[d]thiazol-2-ylcarbamoyl)-3,4-dihydroisoquinolin-2(1H)-yl)-3-(3-(((1s,4r)- |
| | 4-(3-(4-(3-(2,6-dioxopiperidin-3-yl)-1-methyl-1H-indazol-7-yl)piperazin-1- |
| | yl)propyl)cyclohexyl)oxy)-2-methylphenyl)picolinic acid |
| 147b | 6-(8-(benzo[d]thiazol-2-ylcarbamoyl)-3,4-dihydroisoquinolin-2(1H)-yl)-3-(3-(((1r,4s)- |
| | 4-(3-(4-(3-(2,6-dioxopiperidin-3-yl)-1-methyl-1H-indazol-7-yl)piperazin-1- |
| | yl)propyl)cyclohexyl)oxy)-2-methylphenyl)picolinic acid |
| 148 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-6-yl]piperazin-1- |
| | yl]propyl]cyclohexoxy]-2-methyl-phenyl]pyridine-2-carboxylic acid |
| 148a | 6-(8-(benzo[d]thiazol-2-ylcarbamoyl)-3,4-dihydroisoquinolin-2(1H)-yl)-3-(3-(((1s,4r)- |
| | 4-(3-(4-(3-(2,6-dioxopiperidin-3-yl)-1-methyl-1H-indazol-6-yl)piperazin-1- |
| | yl)propyl)cyclohexyl)oxy)-2-methylphenyl)picolinic acid |
| 148b | 6-(8-(benzo[d]thiazol-2-ylcarbamoyl)-3,4-dihydroisoquinolin-2(1H)-yl)-3-(3-(((1r,4s)- |
| | 4-(3-(4-(3-(2,6-dioxopiperidin-3-yl)-1-methyl-1H-indazol-6-yl)piperazin-1- |
| | yl)propyl)cyclohexyl)oxy)-2-methylphenyl)picolinic acid |
| 149 | 6-(8-(benzo[d]thiazol-2-ylcarbamoyl)-3,4-dihydroisoquinolin-2(1H)-yl)-3-(3-(4-(3-(4- |
| | (3-(2,6-dioxopiperidin-3-yl)-1-methyl-1H-indazol-6-yl)piperazin-1- |
| | yl)propyl)phenoxy)-2-methylphenyl)picolinic acid |
| 150 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-7-yl]piperazin-1-yl]propyl]phenoxy]- |
| | 2-methyl-phenyl]pyridine-2-carboxylic acid |
| 151 | 6-(8-(Benzo[d]thiazol-2-ylcarbamoyl)-3,4-dihydroisoquinolin-2(1H)-yl)-3-(3-(3-(4-(2- |
| | (4-(3-(2,6-dioxopiperidin-3-yl)-1-methyl-1H-indazol-7-yl)piperazin-1-yl)-2- |
| | oxoethyl)piperazin-1-yl)propoxy)-2-methylphenyl)picolinic acid |
| 152 | 6-(8-(Benzo[d]thiazol-2-ylcarbamoyl)-3,4-dihydroisoquinolin-2(1H)-yl)-3-(4-(3-(4-(2- |
| | (4-(3-(2,6-dioxopiperidin-3-yl)-1-methyl-1H-indazol-7-yl)piperazin-1-yl)-2- |
| | oxoethyl)piperazin-1-yl)propoxy)-2-methylphenyl)picolinic acid |
| 153 | 6-(8-(Benzo[d]thiazol-2-ylcarbamoyl)-3,4-dihydroisoquinolin-2(1H)-yl)-3-(4-(3-(4-(2- |
| | (4-(3-(2,6-dioxopiperidin-3-yl)-1-methyl-1H-indazol-6-yl)piperazin-1-yl)-2- |
| | oxoethyl)piperazin-1-yl)propoxy)-2-methylphenyl)picolinic acid |
| 154 | 6-[8-(1,3-benzothiazol-2-ylcarbamoyl)-3,4-dihydro-1H-isoquinolin-2-yl]-3-[4-[3-[4- |
| | [2-[4-[3-(2,6-dioxo-3-piperidyl)-1-methyl-indazol-6-yl]piperazin-1-yl]-2-oxo-ethyl]- |
| | 3,5-dimethyl-piperazin-1-yl]propoxy]-2-methyl-phenyl]pyridine-2-carboxylic acid |
| 154a | 6-(8-(Benzo[d]thiazol-2-ylcarbamoyl)-3,4-dihydroisoquinolin-2(1H)-yl)-3-(4-(3- |
| | ((3R,5S)-4-(2-(4-(3-(2,6-dioxopiperidin-3-yl)-1-methyl-1H-indazol-6-yl)piperazin-1- |
| | yl)-2-oxoethyl)-3,5-dimethylpiperazin-1-yl)propoxy)-2-methylphenyl)picolinic acid |
| 155 | 6-[8-(1,3-benzothiazol-2-ylcarbamoyl)-3,4-dihydro-1H-isoquinolin-2-yl]-3-[3-[9-[4- |
| | [3-(2,6-dioxo-3-piperidyl)-1-methyl-indazol-6-yl]piperazin-1-yl]nonoxy]-2-methyl- |
| | phenyl]pyridine-2-carboxylic acid |
| 156 | 6-(8-(benzo[d]thiazol-2-ylcarbamoyl)-3,4-dihydroisoquinolin-2(1H)-yl)-3-(3-((7-(2- |
| | ((3-(2,6-dioxopiperidin-3-yl)-1-methyl-1H-indazol-6-yl)amino)-2-oxoethyl)-7- |
| | azaspiro[3.5]nonan-2-yl)methoxy)-2-methylphenyl)picolinic acid |
| 157 | 2-(5-(1-(((3r,5r,7r)-adamantan-1-yl)methyl)-5-methyl-1H-pyrazol-4-yl)-6-((8-((3-(2,6- |
| | dioxopiperidin-3-yl)-1-methyl-1H-indazol-6-yl)amino)-8-oxooctyl)carbamoyl)pyridin- |
| | 2-yl)-N-(benzo[d]thiazol-2-yl)-1,2,3,4-tetrahydroisoquinoline-8-carboxamide |
| 158 | 6-(8-(benzo[d]thiazol-2-ylcarbamoyl)-3,4-dihydroisoquinolin-2(1H)-yl)-3-(3-(3-(1-(2- |
| | ((3-(2,6-dioxopiperidin-3-yl)-1-methyl-1H-indazol-7-yl)amino)-2-oxoethyl)piperidin- |
| | 4-yl)propoxy)-2-methylphenyl)picolinic acid |
| 159 | 6-[8-(1,3-benzothiazol-2-ylcarbamoyl)-3,4-dihydro-1H-isoquinolin-2-yl]-3-[3-[2-[4- |
| | [2-[[3-(2,6-dioxo-3-piperidyl)-1-methyl-indazol-6-yl]amino]-2-oxo-ethyl]-3,5- |
| | dimethyl-piperazin-1-yl]ethoxy]-2-methyl-phenyl]pyridine-2-carboxylic acid |
| 159a | 6-(8-(benzo[d]thiazol-2-ylcarbamoyl)-3,4-dihydroisoquinolin-2(1H)-yl)-3-(3-(2- |
| | ((3R,5S)-4-(2-((3-(2,6-dioxopiperidin-3-yl)-1-methyl-1H-indazol-6-yl)amino)-2- |
| | oxoethyl)-3,5-dimethylpiperazin-1-yl)ethoxy)-2-methylphenyl)picolinic acid |
| 160 | 6-[8-(1,3-benzothiazol-2-ylcarbamoyl)-3,4-dihydro-1H-isoquinolin-2-yl]-3-[3-[3-[1- |
| | [2-[[3-(2,6-dioxo-3-piperidyl)-1-methyl-indazol-6-yl]amino]-2-oxo-ethyl]pyrrolidin-3- |
| | yl]propoxy]-2-methyl-phenyl]pyridine-2-carboxylic acid |
| 160a | 6-[8-(1,3-benzothiazol-2-ylcarbamoyl)-3,4-dihydro-1H-isoquinolin-2-yl]-3-[3-[3- |
| | [(3R)-1-[2-[[3-(2,6-dioxo-3-piperidyl)-1-methyl-indazol-6-yl]amino]-2-oxo- |
| | ethyl]pyrrolidin-3-yl]propoxy]-2-methyl-phenyl]pyridine-2-carboxylic acid |
| 160b | 6-(8-(benzo[d]thiazol-2-ylcarbamoyl)-3,4-dihydroisoquinolin-2(1H)-yl)-3-(3-(3-((3S)- |
| | 1-(2-((3-(2,6-dioxopiperidin-3-yl)-1-methyl-1H-indazol-6-yl)amino)-2- |
| | oxoethyl)pyrrolidin-3-yl)propoxy)-2-methylphenyl)picolinic acid |
| 161 | 6-[8-(1,3-benzothiazol-2-ylcarbamoyl)-3,4-dihydro-1H-isoquinolin-2-yl]-3-[3-[4-[4- |
| | [4-[3-(2,6-dioxo-3-piperidyl)-1-methyl-indazol-6-yl]piperazin-1- |
| | yl]butyl]cyclohexoxy]-2-methyl-phenyl]pyridine-2-carboxylic acid |
| 161a | 6-(8-(benzo[d]thiazol-2-ylcarbamoyl)-3,4-dihydroisoquinolin-2(1H)-yl)-3-(3-(((1s,4r)- |
| | 4-(4-(4-(3-(2,6-dioxopiperidin-3-yl)-1-methyl-1H-indazol-6-yl)piperazin-1- |
| | yl)butyl)cyclohexyl)oxy)-2-methylphenyl)picolinic acid |
| 162 | 6-[8-(1,3-benzothiazol-2-ylcarbamoyl)-3,4-dihydro-1H-isoquinolin-2-yl]-3-[3-[3-[1- |
| | [2-[[3-(2,6-dioxo-3-piperidyl)-1-methyl-indazol-6-yl]amino]-2-oxo-ethyl]-4- |
| | piperidyl]-1-methyl-propoxy]-2-methyl-phenyl]pyridine-2-carboxylic acid |
| 162a | 6-[8-(1,3-benzothiazol-2-ylcarbamoyl)-3,4-dihydro-1H-isoquinolin-2-yl]-3-[3-[(1S)-3- |
| | [1-[2-[[3-(2,6-dioxo-3-piperidyl)-1-methyl-indazol-6-yl]amino]-2-oxo-ethyl]-4- |
| | piperidyl]-1-methyl-propoxy]-2-methyl-phenyl]pyridine-2-carboxylic acid |
| 162b | 6-[8-(1,3-benzothiazol-2-ylcarbamoyl)-3,4-dihydro-1H-isoquinolin-2-yl]-3-[3-[(1R)-3- |
| | [1-[2-[[3-(2,6-dioxo-3-piperidyl)-1-methyl-indazol-6-yl]amino]-2-oxo-ethyl]-4- |
| | piperidyl]-1-methyl-propoxy]-2-methyl-phenyl]pyridine-2-carboxylic acid |
| 163 | 6-(8-(benzo[d]thiazol-2-ylcarbamoyl)-3,4-dihydroisoquinolin-2(1H)-yl)-3-(3-(4-(4-(4- |
| | (3-(2,6-dioxopiperidin-3-yl)-1-methyl-1H-indazol-6-yl)piperazin-1-yl)butyl)phenoxy)- |
| | 2-methylphenyl)picolinic acid |
| 164 | 6-[8-(1,3-benzothiazol-2-ylcarbamoyl)-3,4-dihydro-1H-isoquinolin-2-yl]-3-[3-[3-[1- |
| | [2-[[3-(2,6-dioxo-3-piperidyl)-1-methyl-indazol-6-yl]amino]-1-methyl-2-oxo-ethyl]-4- |
| | piperidyl]propoxy]-2-methyl-phenyl]pyridine-2-carboxylic acid |
| 164a | 6-(8-(benzo[d]thiazol-2-ylcarbamoyl)-3,4-dihydroisoquinolin-2(1H)-yl)-3-(3-(3-(1- |
| | ((2S)-1-((3-(2,6-dioxopiperidin-3-yl)-1-methyl-1H-indazol-6-yl)amino)-1-oxopropan- |
| | 2-yl)piperidin-4-yl)propoxy)-2-methylphenyl)picolinic acid |
| 164b | 6-(8-(benzo[d]thiazol-2-ylcarbamoyl)-3,4-dihydroisoquinolin-2(1H)-yl)-3-(3-(3-(1- |
| | ((2R)-1-((3-(2,6-dioxopiperidin-3-yl)-1-methyl-1H-indazol-6-yl)amino)-1-oxopropan- |
| | 2-yl)piperidin-4-yl)propoxy)-2-methylphenyl)picolinic acid |
| 165 | 6-(8-(benzo[d]thiazol-2-ylcarbamoyl)-3,4-dihydroisoquinolin-2(1H)-yl)-3-(3-(3-(4-(3- |
| | ((3-(2,6-dioxopiperidin-3-yl)-1-methyl-1H-indazol-6-yl)oxy)propyl)-3- |
| | (trifluoromethyl)piperazin-1-yl)propoxy)-2-methylphenyl)picolinic acid |
| 166 | 6-[8-(1,3-benzothiazol-2-ylcarbamoyl)-3,4-dihydro-1H-isoquinolin-2-yl]-3-[3-[3-[1- |
| | [2-[[3-(2,6-dioxo-3-piperidyl)-1-methyl-indazol-6-yl]amino]-1,1-dimethyl-2-oxo- |
| | ethyl]-4-piperidyl]propoxy]-2-methyl-phenyl]pyridine-2-carboxylic acid |
| 167 | 6-[8-(1,3-benzothiazol-2-ylcarbamoyl)-3,4-dihydro-1H-isoquinolin-2-yl]-3-[3-[3-[1- |
| | [2-[[3-(2,6-dioxo-3-piperidyl)-1-methyl-indazol-6-yl]amino]-2-oxo-ethyl]-4- |
| | piperidyl]butoxy]-2-methyl-phenyl]pyridine-2-carboxylic acid |
| 167a | 6-[8-(1,3-benzothiazol-2-ylcarbamoyl)-3,4-dihydro-1H-isoquinolin-2-yl]-3-[3-[(3R)-3- |
| | [1-[2-[[3-(2,6-dioxo-3-piperidyl)-1-methyl-indazol-6-yl]amino]-2-oxo-ethyl]-4- |
| | piperidyl]butoxy]-2-methyl-phenyl]pyridine-2-carboxylic acid |
| 167b | 6-[8-(1,3-benzothiazol-2-ylcarbamoyl)-3,4-dihydro-1H-isoquinolin-2-yl]-3-[3-[(3S)-3- |
| | [1-[2-[[3-(2,6-dioxo-3-piperidyl)-1-methyl-indazol-6-yl]amino]-2-oxo-ethyl]-4- |
| | piperidyl]butoxy]-2-methyl-phenyl]pyridine-2-carboxylic acid |
| 168 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-7-yl]piperazin-1-yl]-2,2-difluoro- |
| | propyl]cyclohexoxy]-2-methyl-phenyl]pyridine-2-carboxylic acid |
| 168a | 6-(8-(benzo[d]thiazol-2-ylcarbamoyl)-3,4-dihydroisoquinolin-2(1H)-yl)-3-(3-(((1r,4r)- |
| | 4-(3-(4-(3-(2,6-dioxopiperidin-3-yl)-1-methyl-1H-indazol-7-yl)piperazin-1-yl)-2,2- |
| | difluoropropyl)cyclohexyl)oxy)-2-methylphenyl)picolinic acid |
| 169 | 6-[8-(1,3-benzothiazol-2-ylcarbamoyl)-3,4-dihydro-1H-isoquinolin-2-yl]-3-[3-[3-[1- |
| | [2-[[3-(2,6-dioxo-3-piperidyl)-1-methyl-indazol-7-yl]amino]-2-oxo-ethyl]-4- |
| | piperidyl]-1-methyl-propoxy]-2-methyl-phenyl]pyridine-2-carboxylic acid |
| 169a | 6-[8-(1,3-benzothiazol-2-ylcarbamoyl)-3,4-dihydro-1H-isoquinolin-2-yl]-3-[3-[(1S)-3- |
| | [1-[2-[[3-(2,6-dioxo-3-piperidyl)-1-methyl-indazol-7-yl]amino]-2-oxo-ethyl]-4- |
| | piperidyl]-1-methyl-propoxy]-2-methyl-phenyl]pyridine-2-carboxylic acid |
| 169b | 6-[8-(1,3-benzothiazol-2-ylcarbamoyl)-3,4-dihydro-1H-isoquinolin-2-yl]-3-[3-[(1R)-3- |
| | [1-[2-[[3-(2,6-dioxo-3-piperidyl)-1-methyl-indazol-7-yl]amino]-2-oxo-ethyl]-4- |
| | piperidyl]-1-methyl-propoxy]-2-methyl-phenyl] pyridine-2-carboxylic acid |
| 170 | 6-[8-(1,3-benzothiazol-2-ylcarbamoyl)-3,4-dihydro-1H-isoquinolin-2-yl]-3-[3-[3-[1- |
| | [2-[[3-(2,6-dioxo-3-piperidyl)-1-methyl-indazol-7-yl]amino]-2-oxo-ethyl]-4- |
| | piperidyl]-2,2-difluoro-propoxy]-2-methyl-phenyl]pyridine-2-carboxylic acid |
| 171 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-7-yl]piperazin-1-yl]-2,2-difluoro-3- |
| | oxo-propyl]cyclohexoxy]-2-methyl-phenyl]pyridine-2-carboxylic acid |
| 171a | 6-(8-(benzo[d]thiazol-2-ylcarbamoyl)-3,4-dihydroisoquinolin-2(1H)-yl)-3-(3-(((1r,4r)- |
| | 4-(3-(4-(3-(2,6-dioxopiperidin-3-yl)-1-methyl-1H-indazol-7-yl)piperazin-1-yl)-2,2- |
| | difluoro-3-oxopropyl)cyclohexyl)oxy)-2-methylphenyl)picolinic acid |
| 172 | 6-(8-(benzo[d]thiazol-2-ylcarbamoyl)-3,4-dihydroisoquinolin-2(1H)-yl)-3-(3-(3-(1-(3- |
| | ((3-(2,6-dioxopiperidin-3-yl)-1-methyl-1H-indazol-6-yl)oxy)propyl)piperidin-4- |
| | yl)propoxy)-2-methylphenyl)picolinic acid |
| 173 | 6-[8-(1,3-benzothiazol-2-ylcarbamoyl)-3,4-dihydro-1H-isoquinolin-2-yl]-3-[3-[3-[1- |
| | [2-[[3-(2,6-dioxo-3-piperidyl)-1-methyl-indazol-7-yl]amino]-2-oxo-ethyl]-4- |
| | piperidyl]butoxy]-2-methyl-phenyl]pyridine-2-carboxylic acid |
| 173a | 6-(8-(benzo[d]thiazol-2-ylcarbamoyl)-3,4-dihydroisoquinolin-2(1H)-yl)-3-(3-((3S)-3- |
| | (1-(2-((3-(2,6-dioxopiperidin-3-yl)-1-methyl-1H-indazol-7-yl)amino)-2- |
| | oxoethyl)piperidin-4-yl)butoxy)-2-methylphenyl)picolinic acid |
| 173b | 6-[8-(1,3-benzothiazol-2-ylcarbamoyl)-3,4-dihydro-1H-isoquinolin-2-yl]-3-[3-[(3R)-3- |
| | [1-[2-[[3-(2,6-dioxo-3-piperidyl)-1-methyl-indazol-7-yl]amino]-2-oxo-ethyl]-4- |
| | piperidyl]butoxy]-2-methyl-phenyl]pyridine-2-carboxylic acid |
| 174 | 6-[8-(1,3-benzothiazol-2-ylcarbamoyl)-3,4-dihydro-1H-isoquinolin-2-yl]-3-[3-[2-[[8- |
| | [2-[[3-(2,6-dioxo-3-piperidyl)-1-methyl-indazol-6-yl]amino]-2-oxo-ethyl]-8- |
| | azabicyclo[3.2.1]octan-3-yl]oxy]ethoxy]-2-methyl-phenyl]pyridine-2-carboxylic acid |
| 174a | 6-(8-(benzo[d]thiazol-2-ylcarbamoyl)-3,4-dihydroisoquinolin-2(1H)-yl)-3-(3-(2- |
| | (((1R,3r,5S)-8-(2-((3-(2,6-dioxopiperidin-3-yl)-1-methyl-1H-indazol-6-yl)amino)-2- |
| | oxoethyl)-8-azabicyclo[3.2.1]octan-3-yl)oxy)ethoxy)-2-methylphenyl)picolinic acid |
| 174b | 6-(8-(benzo[d]thiazol-2-ylcarbamoyl)-3,4-dihydroisoquinolin-2(1H)-yl)-3-(3-(2- |
| | (((1R,3s,5S)-8-(2-((3-(2,6-dioxopiperidin-3-yl)-1-methyl-1H-indazol-6-yl)amino)-2- |
| | oxoethyl)-8-azabicyclo[3.2.1]octan-3-yl)oxy)ethoxy)-2-methylphenyl)picolinic acid |
| 175 | 6-(8-(benzo[d]thiazol-2-ylcarbamoyl)-3,4-dihydroisoquinolin-2(1H)-yl)-3-(3-(4-(4-(4- |
| | (3-(2,6-dioxopiperidin-3-yl)-1-methyl-1H-indazol-7-yl)piperazin-1-yl)butyl)phenoxy)- |
| | 2-methylphenyl)picolinic acid |
| 176 | 6-[8-(1,3-benzothiazol-2-ylcarbamoyl)-3,4-dihydro-1H-isoquinolin-2-yl]-3-[3-[4-[2- |
| | [4-[3-(2,6-dioxo-3-piperidyl)-1-methyl-indazol-6-yl]piperazin-1-yl]-2-oxo- |
| | ethyl]cyclohexoxy]-2-methyl-phenyl]pyridine-2-carboxylic acid |
| 176a | 6-(8-(benzo[d]thiazol-2-ylcarbamoyl)-3,4-dihydroisoquinolin-2(1H)-yl)-3-(3-(((1r,4r)- |
| | 4-(2-(4-(3-(2,6-dioxopiperidin-3-yl)-1-methyl-1H-indazol-6-yl)piperazin-1-yl)-2- |
| | oxoethyl)cyclohexyl)oxy)-2-methylphenyl)picolinic acid |
| 177 | 6-[8-(1,3-benzothiazol-2-ylcarbamoyl)-3,4-dihydro-1H-isoquinolin-2-yl]-3-[3-[3-[4- |
| | [2-[[3-(2,6-dioxo-3-piperidyl)-1-methyl-indazol-6-yl]amino]-2-oxo-ethyl]-3,5- |
| | dimethyl-piperazin-1-yl]-2,2-difluoro-propoxy]-2-methyl-phenyl]pyridine-2- |
| | carboxylic acid |
| 177a | 6-[8-(1,3-benzothiazol-2-ylcarbamoyl)-3,4-dihydro-1H-isoquinolin-2-yl]-3-[3-[3- |
| | [(3R,5S)-4-[2-[[3-(2,6-dioxo-3-piperidyl)-1-methyl-indazol-6-yl]amino]-2-oxo-ethyl]- |
| | 3,5-dimethyl-piperazin-1-yl]-2,2-difluoro-propoxy]-2-methyl-phenyl]pyridine-2- |
| | carboxylic acid |
| 178 | 6-(8-(benzo[d]thiazol-2-ylcarbamoyl)-3,4-dihydroisoquinolin-2(1H)-yl)-3-(3-(4-(4-(4- |
| | (3-(2,6-dioxopiperidin-3-yl)-1-methyl-1H-indazol-6-yl)piperazin-1-yl)-4- |
| | oxobutyl)phenoxy)-2-methylphenyl)picolinic acid |
| 179 | 6-[8-(1,3-benzothiazol-2-ylcarbamoyl)-3,4-dihydro-1H-isoquinolin-2-yl]-3-[3-[3-[4- |
| | [2-[4-[3-(2,6-dioxo-3-piperidyl)-1-methyl-indazol-7-yl]piperazin-1-yl]-2-oxo-ethyl]- |
| | 3,5-dimethyl-piperazin-1-yl]-2,2-difluoro-propoxy]-2-methyl-phenyl]pyridine-2- |
| | carboxylic acid |
| 179a | 6-[8-(1,3-benzothiazol-2-ylcarbamoyl)-3,4-dihydro-1H-isoquinolin-2-yl]-3-[3-[3- |
| | [(3R,5S)-4-[2-[4-[3-(2,6-dioxo-3-piperidyl)-1-methyl-indazol-7-yl]piperazin-1-yl]-2- |
| | oxo-ethyl]-3,5-dimethyl-piperazin-1-yl]-2,2-difluoro-propoxy]-2-methyl- |
| | phenyl]pyridine-2-carboxylic acid |
| 180 | 6-[8-(1,3-benzothiazol-2-ylcarbamoyl)-3,4-dihydro-1H-isoquinolin-2-yl]-3-[3-[2-[1- |
| | [2-[[3-(2,6-dioxo-3-piperidyl)-1-methyl-indazol-6-yl]amino]-2-oxo-ethyl]-3- |
| | piperidyl]ethoxy]-2-methyl-phenyl]pyridine-2-carboxylic acid |
| 180a | 6-(8-(benzo[d]thiazol-2-ylcarbamoyl)-3,4-dihydroisoquinolin-2(1H)-yl)-3-(3-(2-((3S)- |
| | 1-(2-((3-(2,6-dioxopiperidin-3-yl)-1-methyl-1H-indazol-6-yl)amino)-2- |
| | oxoethyl)piperidin-3-yl)ethoxy)-2-methylphenyl)picolinic acid |
| 180b | 6-(8-(benzo[d]thiazol-2-ylcarbamoyl)-3,4-dihydroisoquinolin-2(1H)-yl)-3-(3-(2-((3R)- |
| | 1-(2-((3-(2,6-dioxopiperidin-3-yl)-1-methyl-1H-indazol-6-yl)amino)-2- |
| | oxoethyl)piperidin-3-yl)ethoxy)-2-methylphenyl)picolinic acid |
| 181 | 6-[8-(1,3-benzothiazol-2-ylcarbamoyl)-3,4-dihydro-1H-isoquinolin-2-yl]-3-[3-[3-[1- |
| | [2-[[3-(2,6-dioxo-3-piperidyl)-1-methyl-indazol-6-yl]amino]-2-oxo-ethyl]-4- |
| | piperidyl]-2,2-difluoro-propoxy]-2-methyl-phenyl]pyridine-2-carboxylic acid |
| 182 | 6-[8-(1,3-benzothiazol-2-ylcarbamoyl)-3,4-dihydro-1H-isoquinolin-2-yl]-3-[3-[4-[2- |
| | [4-[3-(2,6-dioxo-3-piperidyl)-1-methyl-indazol-7-yl]piperazin-1-yl]-2-oxo- |
| | ethyl]cyclohexoxy]-2-methyl-phenyl]pyridine-2-carboxylic acid |
| 182a | 6-(8-(benzo[d]thiazol-2-ylcarbamoyl)-3,4-dihydroisoquinolin-2(1H)-yl)-3-(3-(((1r,4r)- |
| | 4-(2-(4-(3-(2,6-dioxopiperidin-3-yl)-1-methyl-1H-indazol-7-yl)piperazin-1-yl)-2- |
| | oxoethyl)cyclohexyl)oxy)-2-methylphenyl)picolinic acid |
| 183 | 6-[8-(1,3-benzothiazol-2-ylcarbamoyl)-3,4-dihydro-1H-isoquinolin-2-yl]-3-[4-[3-[4- |
| | [2-[4-[3-(2,6-dioxo-3-piperidyl)-1-methyl-indazol-7-yl]piperazin-1-yl]-2-oxo-ethyl]- |
| | 3,5-dimethyl-piperazin-1-yl]propoxy]-2-methyl-phenyl]pyridine-2-carboxylic acid |
| 183a | 6-[8-(1,3-benzothiazol-2-ylcarbamoyl)-3,4-dihydro-1H-isoquinolin-2-yl]-3-[4-[3- |
| | [(3S,5R)-4-[2-[4-[3-(2,6-dioxo-3-piperidyl)-1-methyl-indazol-7-yl]piperazin-1-yl]-2- |
| | oxo-ethyl]-3,5-dimethyl-piperazin-1-yl]propoxy]-2-methyl-phenyl]pyridine-2- |
| | carboxylic acid |
| 184 | 6-[8-(1,3-benzothiazol-2-ylcarbamoyl)-3,4-dihydro-1H-isoquinolin-2-yl]-3-[4-[3-[1- |
| | [2-[[3-(2,6-dioxo-3-piperidyl)-1-methyl-indazol-6-yl]amino]-2-oxo-ethyl]-4- |
| | piperidyl]propoxy]-2-methyl-phenyl]pyridine-2-carboxylic acid |
| 185 | 6-[8-(1,3-benzothiazol-2-ylcarbamoyl)-3,4-dihydro-1H-isoquinolin-2-yl]-3-[3-[3-[4- |
| | [2-[4-[3-(2,6-dioxo-3-piperidyl)-1-methyl-indazol-6-yl]piperazin-1-yl]-2-oxo-ethyl]- |
| | 3,5-dimethyl-piperazin-1-yl]-2,2-difluoro-propoxy]-2-methyl-phenyl]pyridine-2- |
| | carboxylic acid |
| 185a | 6-[8-(1,3-benzothiazol-2-ylcarbamoyl)-3,4-dihydro-1H-isoquinolin-2-yl]-3-[3-[3- |
| | [(3R,5S)-4-[2-[4-[3-(2,6-dioxo-3-piperidyl)-1-methyl-indazol-6-yl]piperazin-1-yl]-2- |
| | oxo-ethyl]-3,5-dimethyl-piperazin-1-yl]-2,2-difluoro-propoxy]-2-methyl- |
| | phenyl]pyridine-2-carboxylic acid |
| 186 | 6-[8-(1,3-benzothiazol-2-ylcarbamoyl)-3,4-dihydro-1H-isoquinolin-2-yl]-3-[3-[4-[1- |
| | [2-[[3-(2,6-dioxo-3-piperidyl)-1-methyl-indazol-7-yl]amino]-2-oxo-ethyl]-3- |
| | piperidyl]butoxy]-2-methyl-phenyl]pyridine-2-carboxylic acid |
| 186a | 6-[8-(1,3-benzothiazol-2-ylcarbamoyl)-3,4-dihydro-1H-isoquinolin-2-yl]-3-[3-[4- |
| | [(3R)-1-[2-[[3-(2,6-dioxo-3-piperidyl)-1-methyl-indazol-7-yl]amino]-2-oxo-ethyl]-3- |
| | piperidyl]butoxy]-2-methyl-phenyl]pyridine-2-carboxylic acid |
| 186b | 6-[8-(1,3-benzothiazol-2-ylcarbamoyl)-3,4-dihydro-1H-isoquinolin-2-yl]-3-[3-[4- |
| | [(3S)-1-[2-[[3-(2,6-dioxo-3-piperidyl)-1-methyl-indazol-7-yl]amino]-2-oxo-ethyl]-3- |
| | piperidyl]butoxy]-2-methyl-phenyl]pyridine-2-carboxylic acid |
| 187 | 6-[8-(1,3-benzothiazol-2-ylcarbamoyl)-3,4-dihydro-1H-isoquinolin-2-yl]-3-[3-[3-[4- |
| | [2-[[3-(2,6-dioxo-3-piperidyl)-1-methyl-indazol-6-yl]amino]-2-oxo-ethyl]-3- |
| | (trifluoromethyl)piperazin-1-yl]propoxy]-2-methyl-phenyl]pyridine-2-carboxylic acid |
| 187a | 6-(8-(benzo[d]thiazol-2-ylcarbamoyl)-3,4-dihydroisoquinolin-2(1H)-yl)-3-(3-(3-((3S)- |
| | 4-(2-((3-(2,6-dioxopiperidin-3-yl)-1-methyl-1H-indazol-6-yl)amino)-2-oxoethyl)-3- |
| | (trifluoromethyl)piperazin-1-yl)propoxy)-2-methylphenyl)picolinic acid |
| 187b | 6-(8-(benzo[d]thiazol-2-ylcarbamoyl)-3,4-dihydroisoquinolin-2(1H)-yl)-3-(3-(3-((3R)- |
| | 4-(2-((3-(2,6-dioxopiperidin-3-yl)-1-methyl-1H-indazol-6-yl)amino)-2-oxoethyl)-3- |
| | (trifluoromethyl)piperazin-1-yl)propoxy)-2-methylphenyl)picolinic acid |
| 188 | 6-[8-(1,3-benzothiazol-2-ylcarbamoyl)-3,4-dihydro-1H-isoquinolin-2-yl]-3-[3-[[7-[2- |
| | [4-[3-(2,6-dioxo-3-piperidyl)-1-methyl-indazol-7-yl]piperazin-1-yl]ethyl]-7- |
| | azaspiro[3.5]nonan-2-yl]methoxy]-2-methyl-phenyl]pyridine-2-carboxylic acid |
| 189 | 6-(8-(benzo[d]thiazol-2-ylcarbamoyl)-3,4-dihydroisoquinolin-2(1H)-yl)-3-(3-(3-(4- |
| | ((1-(3-(2,6-dioxopiperidin-3-yl)-1-methyl-1H-indazol-6-yl)piperidin-4- |
| | yl)methyl)piperazin-1-yl)propoxy)-2-methylphenyl)picolinic acid |
| 190 | 6-(8-(benzo[d]thiazol-2-ylcarbamoyl)-3,4-dihydroisoquinolin-2(1H)-yl)-3-(4-(3-(1-(2- |
| | (4-(3-(2,6-dioxopiperidin-3-yl)-1-methyl-1H-indazol-7-yl)piperazin-1- |
| | yl)ethyl)piperidin-4-yl)propoxy)-2-methylphenyl)picolinic acid |
| 191 | 6-(8-(benzo[d]thiazol-2-ylcarbamoyl)-3,4-dihydroisoquinolin-2(1H)-yl)-3-(3-(4-(4-(4- |
| | (3-(2,6-dioxopiperidin-3-yl)-1-methyl-1H-indazol-7-yl)piperazin-1-yl)-4- |
| | oxobutyl)phenoxy)-2-methylphenyl)picolinic acid |
| 192 | 6-(8-(benzo[d]thiazol-2-ylcarbamoyl)-3,4-dihydroisoquinolin-2(1H)-yl)-3-(3-(3-(1-(2- |
| | (1-(3-(2,6-dioxopiperidin-3-yl)-1-methyl-1H-indazol-6-yl)piperidin-4- |
| | yl)ethyl)piperidin-4-yl)propoxy)-2-methylphenyl)picolinic acid |
| 193 | 6-[8-(1,3-benzothiazol-2-ylcarbamoyl)-3,4-dihydro-1H-isoquinolin-2-yl]-3-[3-[3-[1- |
| | [2-[[3-(2,6-dioxo-3-piperidyl)-1-methyl-indazol-6-yl]amino]-2-oxo-ethyl]-3,3- |
| | difluoro-4-piperidyl]propoxy]-2-methyl-phenyl]pyridine-2-carboxylic acid |
| 193a | 6-(8-(benzo[d]thiazol-2-ylcarbamoyl)-3,4-dihydroisoquinolin-2(1H)-yl)-3-(3-(3-((4R)- |
| | 1-(2-((3-(2,6-dioxopiperidin-3-yl)-1-methyl-1H-indazol-6-yl)amino)-2-oxoethyl)-3,3- |
| | difluoropiperidin-4-yl)propoxy)-2-methylphenyl)picolinic acid |
| 193b | 6-(8-(benzo[d]thiazol-2-ylcarbamoyl)-3,4-dihydroisoquinolin-2(1H)-yl)-3-(3-(3-((4S)- |
| | 1-(2-((3-(2,6-dioxopiperidin-3-yl)-1-methyl-1H-indazol-6-yl)amino)-2-oxoethyl)-3,3- |
| | difluoropiperidin-4-yl)propoxy)-2-methylphenyl)picolinic acid |
| 194 | 6-[8-(1,3-benzothiazol-2-ylcarbamoyl)-3,4-dihydro-1H-isoquinolin-2-yl]-3-[3-[2-[[1- |
| | [2-[[3-(2,6-dioxo-3-piperidyl)-1-methyl-indazol-7-yl]amino]-2-oxo-ethyl]-4- |
| | piperidyl]oxy]ethoxy]-2-methyl-phenyl]pyridine-2-carboxylic acid |
| 195 | 6-[8-(1,3-benzothiazol-2-ylcarbamoyl)-3,4-dihydro-1H-isoquinolin-2-yl]-3-[3-[3-[3- |
| | [4-[3-(2,6-dioxo-3-piperidyl)-1-methyl-indazol-6-yl]piperazin-1-yl]propyl]phenoxy]- |
| | 2-methyl-phenyl]pyridine-2-carboxylic acid |
| 196 | 6-(8-(benzo[d]thiazol-2-ylcarbamoyl)-3,4-dihydroisoquinolin-2(1H)-yl)-3-(3-(3-(1-(2- |
| | ((3-(2,6-dioxopiperidin-3-yl)-1-methyl-1H-indazol-7-yl)amino)-2-oxoethyl)piperidin- |
| | 4-yl)-3,3-difluoropropoxy)-2-methylphenyl)picolinic acid |
| 197 | 6-[8-(1,3-benzothiazol-2-ylcarbamoyl)-3,4-dihydro-1H-isoquinolin-2-yl]-3-[3-[3-[1- |
| | [2-[[3-(2,6-dioxo-3-piperidyl)-1-methyl-indazol-7-yl]amino]-2-oxo-ethyl]-3,3- |
| | difluoro-4-piperidyl]propoxy]-2-methyl-phenyl]pyridine-2-carboxylic acid |
| 197a | 6-(8-(benzo[d]thiazol-2-ylcarbamoyl)-3,4-dihydroisoquinolin-2(1H)-yl)-3-(3-(3-((4S)- |
| | 1-(2-((3-(2,6-dioxopiperidin-3-yl)-1-methyl-1H-indazol-7-yl)amino)-2-oxoethyl)-3,3- |
| | difluoropiperidin-4-yl)propoxy)-2-methylphenyl)picolinic acid |
| 197b | 6-(8-(benzo[d]thiazol-2-ylcarbamoyl)-3,4-dihydroisoquinolin-2(1H)-yl)-3-(3-(3-((4R)- |
| | 1-(2-((3-(2,6-dioxopiperidin-3-yl)-1-methyl-1H-indazol-7-yl)amino)-2-oxoethyl)-3,3- |
| | difluoropiperidin-4-yl)propoxy)-2-methylphenyl)picolinic acid |
| 198 | 6-(8-(benzo[d]thiazol-2-ylcarbamoyl)-3,4-dihydroisoquinolin-2(1H)-yl)-3-(3-(2-(4- |
| | ((1-(3-(2,6-dioxopiperidin-3-yl)-1-methyl-1H-indazol-6-yl)piperidin-4- |
| | yl)methyl)piperazin-1-yl)ethoxy)-2-methylphenyl)picolinic acid |
| 199 | 6-[8-(1,3-benzothiazol-2-ylcarbamoyl)-3,4-dihydro-1H-isoquinolin-2-yl]-3-[4-[3-[1- |
| | [2-[4-[3-(2,6-dioxo-3-piperidyl)-1-methyl-indazol-6-yl]piperazin-1-yl]ethyl]-4- |
| | piperidyl]propoxy]-2-methyl-phenyl]pyridine-2-carboxylic acid |
| 200 | 6-[8-(1,3-benzothiazol-2-ylcarbamoyl)-3,4-dihydro-1H-isoquinolin-2-yl]-3-[3-[3-[4- |
| | [[4-[3-(2,6-dioxo-3-piperidyl)-1-methyl-indazol-6-yl]piperazin-1-yl]methyl]-1- |
| | piperidyl]propoxy]-2-methyl-phenyl] pyridine-2-carboxylic acid |
| 201 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-7-yl]piperazin-1-yl]-2,2-difluoro- |
| | propyl]phenoxy]-2-methyl-phenyl]pyridine-2-carboxylic acid |
| 202 | 6-[8-(1,3-benzothiazol-2-ylcarbamoyl)-3,4-dihydro-1H-isoquinolin-2-yl]-3-[3-[4-[1- |
| | [2-[[3-(2,6-dioxo-3-piperidyl)-1-methyl-indazol-6-yl]amino]-2-oxo-ethyl]-3- |
| | piperidyl]butoxy]-2-methyl-phenyl]pyridine-2-carboxylic acid |
| 202a | 6-[8-(1,3-benzothiazol-2-ylcarbamoyl)-3,4-dihydro-1H-isoquinolin-2-yl]-3-[3-[4- |
| | [(3S)-1-[2-[[3-(2,6-dioxo-3-piperidyl)-1-methyl-indazol-6-yl]amino]-2-oxo-ethyl]-3- |
| | piperidyl]butoxy]-2-methyl-phenyl]pyridine-2-carboxylic acid |
| 202b | 6-[8-(1,3-benzothiazol-2-ylcarbamoyl)-3,4-dihydro-1H-isoquinolin-2-yl]-3-[3-[4- |
| | [(3R)-1-[2-[[3-(2,6-dioxo-3-piperidyl)-1-methyl-indazol-6-yl]amino]-2-oxo-ethyl]-3- |
| | piperidyl]butoxy]-2-methyl-phenyl]pyridine-2-carboxylic acid |
| 203 | 6-(8-(benzo[d]thiazol-2-ylcarbamoyl)-3,4-dihydroisoquinolin-2(1H)-yl)-3-(3-(4-(1-(2- |
| | ((3-(2,6-dioxopiperidin-3-yl)-1-methyl-1H-indazol-6-yl)amino)-2-oxoethyl)piperidin- |
| | 4-yl)butoxy)-2-methylphenyl)picolinic acid |
| 204 | 6-[8-(1,3-benzothiazol-2-ylcarbamoyl)-3,4-dihydro-1H-isoquinolin-2-yl]-3-[3-[4-[4- |
| | [4-[3-(2,6-dioxo-3-piperidyl)-1-methyl-indazol-7-yl]piperazin-1- |
| | yl]butyl]cyclohexoxy]-2-methyl-phenyl]pyridine-2-carboxylic acid |
| 204a | 6-(8-(benzo[d]thiazol-2-ylcarbamoyl)-3,4-dihydroisoquinolin-2(1H)-yl)-3-(3-(((1s,4r)- |
| | 4-(4-(4-(3-(2,6-dioxopiperidin-3-yl)-1-methyl-1H-indazol-7-yl)piperazin-1- |
| | yl)butyl)cyclohexyl)oxy)-2-methylphenyl)picolinic acid |
| 205 | 6-[8-(1,3-benzothiazol-2-ylcarbamoyl)-3,4-dihydro-1H-isoquinolin-2-yl]-3-[3-[8-[4- |
| | [3-(2,6-dioxo-3-piperidyl)-1-methyl-indazol-6-yl]piperazin-1-yl]octoxy]-2-methyl- |
| | phenyl]pyridine-2-carboxylic acid |
| 206 | 6-[8-(1,3-benzothiazol-2-ylcarbamoyl)-3,4-dihydro-1H-isoquinolin-2-yl]-3-[3-[7-[4- |
| | [3-(2,6-dioxo-3-piperidyl)-1-methyl-indazol-7-yl]piperazin-1-yl]heptoxy]-2-methyl- |
| | phenyl]pyridine-2-carboxylic acid |
| 207 | 6-[8-(1,3-benzothiazol-2-ylcarbamoyl)-3,4-dihydro-1H-isoquinolin-2-yl]-3-[3-[3-[1- |
| | [2-[[3-(2,6-dioxo-3-piperidyl)-1-methyl-indazol-6-yl]amino]-2-oxo-ethyl]-3- |
| | piperidyl]propoxy]-2-methyl-phenyl]pyridine-2-carboxylic acid |
| 207a | 6-(8-(benzo[d]thiazol-2-ylcarbamoyl)-3,4-dihydroisoquinolin-2(1H)-yl)-3-(3-(3-((3S)- |
| | 1-(2-((3-(2,6-dioxo piperidin-3-yl)-1-methyl-1H-indazol-6-yl)amino)-2- |
| | oxoethyl)piperidin-3-yl)propoxy)-2-methylphenyl) picolinic acid |
| 207b | 6-(8-(benzo[d]thiazol-2-ylcarbamoyl)-3,4-dihydroisoquinolin-2(1H)-yl)-3-(3-(3-((3R)- |
| | 1-(2-((3-(2,6-dioxopiperidin-3-yl)-1-methyl-1H-indazol-6-yl)amino)-2- |
| | oxoethyl)piperidin-3-yl)propoxy)-2-methylphenyl)picolinic acid |
| 208 | 6-[8-(1,3-benzothiazol-2-ylcarbamoyl)-3,4-dihydro-1H-isoquinolin-2-yl]-3-[3-[4-[2- |
| | [4-[3-(2,6-dioxo-3-piperidyl)-1-methyl-indazol-6-yl]piperazin-1-yl]ethyl]phenoxy]-2- |
| | methyl-phenyl]pyridine-2-carboxylic acid |
| 209 | 6-[8-(1,3-benzothiazol-2-ylcarbamoyl)-3,4-dihydro-1H-isoquinolin-2-yl]-3-[3-[3-[4- |
| | [2-[[3-(2,6-dioxo-3-piperidyl)-1-methyl-indazol-7-yl]amino]-2-oxo-ethyl]-3,5- |
| | dimethyl-piperazin-1-yl]-2,2-difluoro-propoxy]-2-methyl-phenyl]pyridine-2- |
| | carboxylic acid |
| 209a | 6-[8-(1,3-benzothiazol-2-ylcarbamoyl)-3,4-dihydro-1H-isoquinolin-2-yl]-3-[3-[3- |
| | [(3R,5S)-4-[2-[[3-(2,6-dioxo-3-piperidyl)-1-methyl-indazol-7-yl]amino]-2-oxo-ethyl]- |
| | 3,5-dimethyl-piperazin-1-yl]-2,2-difluoro-propoxy]-2-methyl-phenyl]pyridine-2- |
| | carboxylic acid |
| 210 | 6-[8-(1,3-benzothiazol-2-ylcarbamoyl)-3,4-dihydro-1H-isoquinolin-2-yl]-3-[3-[3-[1- |
| | [2-[[3-(2,6-dioxo-3-piperidyl)-1-methyl-indazol-7-yl]amino]-2-oxo-ethyl]-3- |
| | piperidyl]propoxy]-2-methyl-phenyl]pyridine-2-carboxylic acid |
| 210a | 6-(8-(benzo[d]thiazol-2-ylcarbamoyl)-3,4-dihydroisoquinolin-2(1H)-yl)-3-(3-(3-((3S)- |
| | 1-(2-((3-(2,6-dioxopiperidin-3-yl)-1-methyl-1H-indazol-7-yl)amino)-2- |
| | oxoethyl)piperidin-3-yl)propoxy)-2-methylphenyl)picolinic acid |
| 210b | 6-(8-(benzo[d]thiazol-2-ylcarbamoyl)-3,4-dihydroisoquinolin-2(1H)-yl)-3-(3-(3-((3R)- |
| | 1-(2-((3-(2,6-dioxopiperidin-3-yl)-1-methyl-1H-indazol-7-yl)amino)-2- |
| | oxoethyl)piperidin-3-yl)propoxy)-2-methylphenyl)picolinic acid |
| 211 | 6-[8-(1,3-benzothiazol-2-ylcarbamoyl)-3,4-dihydro-1H-isoquinolin-2-yl]-3-[3-[4-[2- |
| | [4-[3-(2,6-dioxo-3-piperidyl)-1-methyl-indazol-6-yl]piperazin-1- |
| | ylethyl]cyclohexoxy]-2-methyl-phenyl]pyridine-2-carboxylic acid |
| 211a | 6-(8-(benzo[d]thiazol-2-ylcarbamoyl)-3,4-dihydroisoquinolin-2(1H)-yl)-3-(3-(((1r,4r)- |
| | 4-(2-(4-(3-(2,6-dioxopiperidin-3-yl)-1-methyl-1H-indazol-6-yl)piperazin-1- |
| | yl)ethyl)cyclohexyl)oxy)-2-methylphenyl)picolinic acid |
| 212 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-6-yl]piperazin-1-yl]-2,2-difluoro- |
| | propyl]cyclohexoxy]-2-methyl-phenyl]pyridine-2-carboxylic acid |
| 212a | 6-(8-(benzo[d]thiazol-2-ylcarbamoyl)-3,4-dihydroisoquinolin-2(1H)-yl)-3-(3-(((1r,4r)- |
| | 4-(3-(4-(3-(2,6-dioxopiperidin-3-yl)-1-methyl-1H-indazol-6-yl)piperazin-1-yl)-2,2- |
| | difluoropropyl)cyclohexyl)oxy)-2-methylphenyl)picolinic acid |
| 213 | 6-(8-(benzo[d]thiazol-2-ylcarbamoyl)-3,4-dihydroisoquinolin-2(1H)-yl)-3-(3-(4-(1-(2- |
| | ((3-(2,6-dioxopiperidin-3-yl)-1-methyl-1H-indazol-7-yl)amino)-2-oxoethyl)piperidin- |
| | 4-yl)butoxy)-2-methylphenyl)picolinic acid |
| 214 | 6-[8-(1,3-benzothiazol-2-ylcarbamoyl)-3,4-dihydro-1H-isoquinolin-2-yl]-3-[3-[4-[4- |
| | [4-[3-(2,6-dioxo-3-piperidyl)-1-methyl-indazol-7-yl]piperazin-1-yl]-4-oxo- |
| | butyl]cyclohexoxy]-2-methyl-phenyl]pyridine-2-carboxylic acid |
| 214a | 6-(8-(benzo[d]thiazol-2-ylcarbamoyl)-3,4-dihydroisoquinolin-2(1H)-yl)-3-(3-(((1s,4r)- |
| | 4-(4-(4-(3-(2,6-dioxopiperidin-3-yl)-1-methyl-1H-indazol-7-yl)piperazin-1-yl)-4- |
| | oxobutyl)cyclohexyl)oxy)-2-methylphenyl)picolinic acid |
| 215 | 6-[8-(1,3-benzothiazol-2-ylcarbamoyl)-3,4-dihydro-1H-isoquinolin-2-yl]-3-[3-[4-[2- |
| | [4-[3-(2,6-dioxo-3-piperidyl)-1-methyl-indazol-7-yl]piperazin-1- |
| | yl]ethyl]cyclohexoxy]-2-methyl-phenyl]pyridine-2-carboxylic acid |
| 215a | 6-(8-(benzo[d]thiazol-2-ylcarbamoyl)-3,4-dihydroisoquinolin-2(1H)-yl)-3-(3-(((1r,4r)- |
| | 4-(2-(4-(3-(2,6-dioxopiperidin-3-yl)-1-methyl-1H-indazol-7-yl)piperazin-1- |
| | yl)ethyl)cyclohexyl)oxy)-2-methylphenyl)picolinic acid |
| 216 | 6-[8-(1,3-benzothiazol-2-ylcarbamoyl)-3,4-dihydro-1H-isoquinolin-2-yl]-3-[3-[3-[1- |
| | [2-[[3-(2,6-dioxo-3-piperidyl)-1-methyl-indazol-7-yl]amino]-1-methyl-2-oxo-ethyl]-4- |
| | piperidyl]propoxy]-2-methyl-phenyl]pyridine-2-carboxylic acid |
| 216a | 6-(8-(benzo[d]thiazol-2-ylcarbamoyl)-3,4-dihydroisoquinolin-2(1H)-yl)-3-(3-(3-(1- |
| | ((2S)-1-((3-(2,6-dioxopiperidin-3-yl)-1-methyl-1H-indazol-7-yl)amino)-1-oxopropan- |
| | 2-yl)piperidin-4-yl)propoxy)-2-methylphenyl)picolinic acid |
| 216b | 6-(8-(benzo[d]thiazol-2-ylcarbamoyl)-3,4-dihydroisoquinolin-2(1H)-yl)-3-(3-(3-(1- |
| | ((2R)-1-((3-(2,6-dioxopiperidin-3-yl)-1-methyl-1H-indazol-7-yl)amino)-1-oxopropan- |
| | 2-yl)piperidin-4-yl)propoxy)-2-methylphenyl)picolinic acid |
| 217 | 6-[8-(1,3-benzothiazol-2-ylcarbamoyl)-3,4-dihydro-1H-isoquinolin-2-yl]-3-[3-[8-[4- |
| | [3-(2,6-dioxo-3-piperidyl)-1-methyl-indazol-7-yl]piperazin-1-yl]octoxy]-2-methyl- |
| | phenyl]pyridine-2-carboxylic acid |
| 218 | 6-[8-(1,3-benzothiazol-2-ylcarbamoyl)-3,4-dihydro-1H-isoquinolin-2-yl]-3-[3-[3-[4- |
| | [2-[[3-(2,6-dioxo-3-piperidyl)-1-methyl-indazol-7-yl]amino]-2-oxo-ethyl]-3- |
| | (trifluoromethyl)piperazin-1-yl]propoxy]-2-methyl-phenyl]pyridine-2-carboxylic acid |
| 218a | 6-(8-(benzo[d]thiazol-2-ylcarbamoyl)-3,4-dihydroisoquinolin-2(1H)-yl)-3-(3-(3-((3R)- |
| | 4-(2-((3-(2,6-dioxopiperidin-3-yl)-1-methyl-1H-indazol-7-yl)amino)-2-oxoethyl)-3- |
| | (trifluoromethyl)piperazin-1-yl)propoxy)-2-methylphenyl)picolinic acid |
| 218b | 6-(8-(benzo[d]thiazol-2-ylcarbamoyl)-3,4-dihydroisoquinolin-2(1H)-yl)-3-(3-(3-((3S)- |
| | 4-(2-((3-(2,6-dioxopiperidin-3-yl)-1-methyl-1H-indazol-7-yl)amino)-2-oxoethyl)-3- |
| | (trifluoromethyl)piperazin-1-yl)propoxy)-2-methylphenyl)picolinic acid |
| 219 | 6-[8-(1,3-benzothiazol-2-ylcarbamoyl)-3,4-dihydro-1H-isoquinolin-2-yl]-3-[3-[2-[1- |
| | [2-[[3-(2,6-dioxo-3-piperidyl)-1-methyl-indazol-6-yl]amino]-2-oxo-ethyl]pyrrolidin-3- |
| | yl]ethoxy]-2-methyl-phenyl]pyridine-2-carboxylic acid |
| 219a | 6-[8-(1,3-benzothiazol-2-ylcarbamoyl)-3,4-dihydro-1H-isoquinolin-2-yl]-3-[3-[2- |
| | [(3S)-1-[2-[[3-(2,6-dioxo-3-piperidyl)-1-methyl-indazol-6-yl]amino]-2-oxo- |
| | ethyl]pyrrolidin-3-yl]ethoxy]-2-methyl-phenyl]pyridine-2-carboxylic acid |
| 219b | 6-[8-(1,3-benzothiazol-2-ylcarbamoyl)-3,4-dihydro-1H-isoquinolin-2-yl]-3-[3-[2- |
| | [(3R)-1-[2-[[3-(2,6-dioxo-3-piperidyl)-1-methyl-indazol-6-yl]amino]-2-oxo- |
| | ethyl]pyrrolidin-3-yl]ethoxy]-2-methyl-phenyl]pyridine-2-carboxylic acid |
| 220 | 6-[8-(1,3-benzothiazol-2-ylcarbamoyl)-3,4-dihydro-1H-isoquinolin-2-yl]-3-[3-[3-[1- |
| | [2-[4-[3-(2,6-dioxo-3-piperidyl)-1-methyl-indazol-6-yl]piperazin-1-yl]ethyl]-4- |
| | piperidyl]propoxy]-2-methyl-phenyl]pyridine-2-carboxylic acid |
| 221 | 6-(8-(benzo[d]thiazol-2-ylcarbamoyl)-3,4-dihydroisoquinolin-2(1H)-yl)-3-(3-(3-(4- |
| | ((1-(3-(2,6-dioxopiperidin-3-yl)-1-methyl-1H-indazol-7-yl)piperidin-4- |
| | yl)methyl)piperazin-1-yl)propoxy)-2-methylphenyl)picolinic acid |
| 222 | 6-(8-(benzo[d]thiazol-2-ylcarbamoyl)-3,4-dihydroisoquinolin-2(1H)-yl)-3-(3-((6-(4- |
| | (3-(2,6-dioxopiperidin-3-yl)-1-methyl-1H-indazol-6-yl)piperazin-1-yl)hexyl)oxy)-2- |
| | methylphenyl)picolinic acid |
| 223 | 6-(8-(benzo[d]thiazol-2-ylcarbamoyl)-3,4-dihydroisoquinolin-2(1H)-yl)-3-(3-(2-(1-(2- |
| | (1-(3-(2,6-dioxopiperidin-3-yl)-1-methyl-1H-indazol-6-yl)piperidin-4- |
| | yl)ethyl)piperidin-4-yl)ethoxy)-2-methylphenyl)picolinic acid |
| 224 | 6-[8-(1,3-benzothiazol-2-ylcarbamoyl)-3,4-dihydro-1H-isoquinolin-2-yl]-3-[3-[2-[[8- |
| | [2-[[3-(2,6-dioxo-3-piperidyl)-1-methyl-indazol-7-yl]amino]-2-oxo-ethyl]-8- |
| | azabicyclo[3.2.1]octan-3-yl]oxy]ethoxy]-2-methyl-phenyl]pyridine-2-carboxylic acid |
| 224a | 6-(8-(benzo[d]thiazol-2-ylcarbamoyl)-3,4-dihydroisoquinolin-2(1H)-yl)-3-(3-(2- |
| | (((1R,3s,5S)-8-(2-((3-(2,6-dioxopiperidin-3-yl)-1-methyl-1H-indazol-7-yl)amino)-2- |
| | oxoethyl)-8-azabicyclo[3.2.1]octan-3-yl)oxy)ethoxy)-2-methylphenyl)picolinic acid |
| 224b | 6-(8-(benzo[d]thiazol-2-ylcarbamoyl)-3,4-dihydroisoquinolin-2(1H)-yl)-3-(3-(2- |
| | (((1R,3r,5S)-8-(2-((3-(2,6-dioxopiperidin-3-yl)-1-methyl-1H-indazol-7-yl)amino)-2- |
| | oxoethyl)-8-azabicyclo[3.2.1]octan-3-yl)oxy)ethoxy)-2-methylphenyl)picolinic acid |
| 225 | 6-(8-(benzo[d]thiazol-2-ylcarbamoyl)-3,4-dihydroisoquinolin-2(1H)-yl)-3-(3-((7-(2- |
| | (4-(3-(2,6-dioxopiperidin-3-yl)-1-methyl-1H-indazol-6-yl)piperazin-1-yl)ethyl)-7- |
| | azaspiro[3.5]nonan-2-yl)methoxy)-2-methylphenyl)picolinic acid |
| 226 | 6-[8-(1,3-benzothiazol-2-ylcarbamoyl)-3,4-dihydro-1H-isoquinolin-2-yl]-3-[3-[[7-[2- |
| | [[3-(2,6-dioxo-3-piperidyl)-1-methyl-indazol-7-yl]amino]-2-oxo-ethyl]-7- |
| | azaspiro[3.5]nonan-2-yl]methoxy]-2-methyl-phenyl]pyridine-2-carboxylic acid |
| 227 | 6-[8-(1,3-benzothiazol-2-ylcarbamoyl)-3,4-dihydro-1H-isoquinolin-2-yl]-3-[3-[4-[4- |
| | [4-[3-(2,6-dioxo-3-piperidyl)-1-methyl-indazol-6-yl]piperazin-1-yl]-4-oxo- |
| | butyl]cyclohexoxy]-2-methyl-phenyl]pyridine-2-carboxylic acid |
| 227a | 6-(8-(benzo[d]thiazol-2-ylcarbamoyl)-3,4-dihydroisoquinolin-2(1H)-yl)-3-(3-(((1s,4r)- |
| | 4-(4-(4-(3-(2,6-dioxopiperidin-3-yl)-1-methyl-1H-indazol-6-yl)piperazin-1-yl)-4- |
| | oxobutyl)cyclohexyl)oxy)-2-methylphenyl)picolinic acid |
| 228 | 6-(8-(benzo[d]thiazol-2-ylcarbamoyl)-3,4-dihydroisoquinolin-2(1H)-yl)-3-(3-(2-(4- |
| | ((1-(3-(2,6-dioxopiperidin-3-yl)-1-methyl-1H-indazol-7-yl)piperidin-4- |
| | yl)methyl)piperazin-1-yl)ethoxy)-2-methylphenyl)picolinic acid |
| 229 | 6-(8-(benzo[d]thiazol-2-ylcarbamoyl)-3,4-dihydroisoquinolin-2(1H)-yl)-3-(3-(3-(1-(2- |
| | ((3-(2,6-dioxopiperidin-3-yl)-1-methyl-1H-indazol-6-yl)amino)-2-oxoethyl)piperidin- |
| | 4-yl)-3,3-difluoropropoxy)-2-methylphenyl)picolinic acid |
| 230 | 6-(8-(benzo[d]thiazol-2-ylcarbamoyl)-3,4-dihydroisoquinolin-2(1H)-yl)-3-(3-(3-(1-(2- |
| | (4-(3-(2,6-dioxopiperidin-3-yl)-1-methyl-1H-indazol-7-yl)piperazin-1- |
| | yl)ethyl)piperidin-4-yl)propoxy)-2-methylphenyl)picolinic acid |
| 231 | 6-[8-(1,3-benzothiazol-2-ylcarbamoyl)-3,4-dihydro-1H-isoquinolin-2-yl]-3-[3-[2-[1- |
| | [2-[[3-(2,6-dioxo-3-piperidyl)-1-methyl-indazol-7-yl]amino]-2-oxo-ethyl]pyrrolidin-3- |
| | yl]ethoxy]-2-methyl-phenyl]pyridine-2-carboxylic acid |
| 231a | 6-[8-(1,3-benzothiazol-2-ylcarbamoyl)-3,4-dihydro-1H-isoquinolin-2-yl]-3-[3-[2- |
| | [(3S)-1-[2-[[3-(2,6-dioxo-3-piperidyl)-1-methyl-indazol-7-yl]amino]-2-oxo- |
| | ethyl]pyrrolidin-3-yl]ethoxy]-2-methyl-phenyl]pyridine-2-carboxylic acid |
| 231b | 6-[8-(1,3-benzothiazol-2-ylcarbamoyl)-3,4-dihydro-1H-isoquinolin-2-yl]-3-[3-[2- |
| | [(3R)-1-[2-[[3-(2,6-dioxo-3-piperidyl)-1-methyl-indazol-7-yl]amino]-2-oxo- |
| | ethyl]pyrrolidin-3-yl]ethoxy]-2-methyl-phenyl]pyridine-2-carboxylic acid |
| 232 | 6-[8-(1,3-benzothiazol-2-ylcarbamoyl)-3,4-dihydro-1H-isoquinolin-2-yl]-3-[3-[2-[1- |
| | [2-[4-[3-(2,6-dioxo-3-piperidyl)-1-methyl-indazol-6-yl]piperazin-1-yl]ethyl]-4- |
| | piperidyl]ethoxy]-2-methyl-phenyl]pyridine-2-carboxylic acid |
| 233 | 6-[8-(1,3-benzothiazol-2-ylcarbamoyl)-3,4-dihydro-1H-isoquinolin-2-yl]-3-[3-[2-[1- |
| | [2-[4-[3-(2,6-dioxo-3-piperidyl)-1-methyl-indazol-7-yl]piperazin-1-yl]ethyl]-4- |
| | piperidyl]ethoxy]-2-methyl-phenyl]pyridine-2-carboxylic acid |
| 234 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-6-yl]piperazin-1-yl]-2,2-difluoro- |
| | propyl]phenoxy]-2-methyl-phenyl]pyridine-2-carboxylic acid |
| 235 | 6-[8-(1,3-benzothiazol-2-ylcarbamoyl)-3,4-dihydro-1H-isoquinolin-2-yl]-3-[3-[4-[4- |
| | [4-[3-(2,6-dioxo-3-piperidyl)-1-methyl-indazol-6-yl]piperazin-1-yl]methyl]-1- |
| | piperidyl]butoxy]-2-methyl-phenyl]pyridine-2-carboxylic acid |
| 236 | 6-(8-(Benzo[d]thiazol-2-ylcarbamoyl)-3,4-dihydroisoquinolin-2(1H)-yl)-3-(4-(3-(1-(2- |
| | ((3-(2,6-dioxopiperidin-3-yl)-1-methyl-1H-indazol-7-yl)amino)-2-oxoethyl)piperidin- |
| | 4-yl)propoxy)-2-methylphenyl)picolinic acid |
| 237 | 6-[8-(1,3-benzothiazol-2-ylcarbamoyl)-3,4-dihydro-1H-isoquinolin-2-yl]-3-[3-[9-[4- |
| | [3-(2,6-dioxo-3-piperidyl)-1-methyl-indazol-7-yl]piperazin-1-yl]nonoxy]-2-methyl- |
| | phenyl]pyridine-2-carboxylic acid |
| 238 | 6-(8-(benzo[d]thiazol-2-ylcarbamoyl)-3,4-dihydroisoquinolin-2(1H)-yl)-3-(3-(2-((1- |
| | (2-((3-(2,6-dioxopiperidin-3-yl)-1-methyl-1H-indazol-6-yl)amino)-2- |
| | oxoethyl)piperidin-4-yl)oxy)ethoxy)-2-methylphenyl)picolinic acid |
| 239 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-6-yl]piperazin-1-yl]-2,2-difluoro-3- |
| | oxo-propyl]cyclohexoxy]-2-methyl-phenyl]pyridine-2-carboxylic acid |
| 239a | 6-(8-(benzo[d]thiazol-2-ylcarbamoyl)-3,4-dihydroisoquinolin-2(1H)-yl)-3-(3-(((1r,4r)- |
| | 4-(3-(4-(3-(2,6-dioxopiperidin-3-yl)-1-methyl-1H-indazol-6-yl)piperazin-1-yl)-2,2- |
| | difluoro-3-oxopropyl)cyclohexyl)oxy)-2-methylphenyl)picolinic acid |
| 240 | 6-[8-(1,3-benzothiazol-2-ylcarbamoyl)-3,4-dihydro-1H-isoquinolin-2-yl]-3-[3-[3-[1- |
| | [2-[[3-(2,6-dioxo-3-piperidyl)-1-methyl-indazol-7-yl]amino]-2-oxo-ethyl]pyrrolidin-3- |
| | yl]propoxyl-2-methyl-phenyl]pyridine-2-carboxylic acid |
| 240a | 6-[8-(1,3-benzothiazol-2-ylcarbamoyl)-3,4-dihydro-1H-isoquinolin-2-yl]-3-[3-[3- |
| | [(3R)-1-[2-[[3-(2,6-dioxo-3-piperidyl)-1-methyl-indazol-7-yl]amino]-2-oxo- |
| | ethyl]pyrrolidin-3-yl]propoxy]-2-methyl-phenyl]pyridine-2-carboxylic acid |
| 240b | 6-(8-(benzo[d]thiazol-2-ylcarbamoyl)-3,4-dihydroisoquinolin-2(1H)-yl)-3-(3-(3-((3S)- |
| | 1-(2-((3-(2,6-dioxopiperidin-3-yl)-1-methyl-1H-indazol-7-yl)amino)-2- |
| | oxoethyl)pyrrolidin-3-yl)propoxy)-2-methylphenyl)picolinic acid |
| 241 | 6-[8-(1,3-benzothiazol-2-ylcarbamoyl)-3,4-dihydro-1H-isoquinolin-2-yl]-3-[3-[4-[2- |
| | [4-[3-(2,6-dioxo-3-piperidyl)-1-methyl-indazol-7-yl]piperazin-1-yl]ethyl]phenoxy]-2- |
| | methyl-phenyl]pyridine-2-carboxylic acid |
| 242 | 6-[8-(1,3-benzothiazol-2-ylcarbamoyl)-3,4-dihydro-1H-isoquinolin-2-yl]-3-[4-[3-[4- |
| | [2-[[3-(2,6-dioxo-3-piperidyl)-1-methyl-indazol-7-yl]amino]-2-oxo-ethyl]piperazin-1- |
| | yl]propoxy]-2-methyl-phenyl]pyridine-2-carboxylic acid |
| 243 | 6-(8-(benzo[d]thiazol-2-ylcarbamoyl)-3,4-dihydroisoquinolin-2(1H)-yl)-3-(3-(2-(3- |
| | ((3-(2,6-dioxopiperidin-3-yl)-1-methyl-1H-indazol-7-yl)amino)-3- |
| | oxopropyl)phenoxy)-2-methylphenyl)picolinic acid |
| 244 | 6-(8-(benzo[d]thiazol-2-ylcarbamoyl)-3,4-dihydroisoquinolin-2(1H)-yl)-3-(3-(4-(3-(4- |
| | (3-(2,6-dioxopiperidin-3-yl)-1-methyl-1H-indazol-6-yl)piperazin-1-yl)-3- |
| | oxopropyl)phenoxy)-2-methylphenyl)picolinic acid |
| 245 | 6-[8-(1,3-benzothiazol-2-ylcarbamoyl)-3,4-dihydro-1H-isoquinolin-2-yl]-3-[3-[3-[4- |
| | [2-[[3-(2,6-dioxo-3-piperidyl)-1-methyl-indazol-6-yl]amino]-2-oxo-ethyl]-2- |
| | (trifluoromethyl)piperazin-1-yl]propoxy]-2-methyl-phenyl]pyridine-2-carboxylic acid |
| 245a | 6-(8-(benzo[d]thiazol-2-ylcarbamoyl)-3,4-dihydroisoquinolin-2(1H)-yl)-3-(3-(3-((2R)- |
| | 4-(2-((3-(2,6-dioxopiperidin-3-yl)-1-methyl-1H-indazol-6-yl)amino)-2-oxoethyl)-2- |
| | (trifluoromethyl)piperazin-1-yl)propoxy)-2-methylphenyl)picolinic acid |
| 245b | 6-(8-(benzo[d]thiazol-2-ylcarbamoyl)-3,4-dihydroisoquinolin-2(1H)-yl)-3-(3-(3-((2S)- |
| | 4-(2-((3-(2,6-dioxopiperidin-3-yl)-1-methyl-1H-indazol-6-yl)amino)-2-oxoethyl)-2- |
| | (trifluoromethyl)piperazin-1-yl)propoxy)-2-methylphenyl)picolinic acid |
| 246 | 6-[8-(1,3-benzothiazol-2-ylcarbamoyl)-3,4-dihydro-1H-isoquinolin-2-yl]-3-[3-[3-[4- |
| | [2-[[3-(2,6-dioxo-3-piperidyl)-1-methyl-indazol-7-yl]amino]-2-oxo-ethyl]-2- |
| | (trifluoromethyl)piperazin-1-yl]propoxy]-2-methyl-phenyl]pyridine-2-carboxylic acid |
| 246a | 6-(8-(benzo[d]thiazol-2-ylcarbamoyl)-3,4-dihydroisoquinolin-2(1H)-yl)-3-(3-(3-((2R)- |
| | 4-(2-((3-(2,6-dioxopiperidin-3-yl)-1-methyl-1H-indazol-7-yl)amino)-2-oxoethyl)-2- |
| | (trifluoromethyl)piperazin-1-yl)propoxy)-2-methylphenyl)picolinic acid |
| 246b | 6-(8-(benzo[d]thiazol-2-ylcarbamoyl)-3,4-dihydroisoquinolin-2(1H)-yl)-3-(3-(3-((2S)- |
| | 4-(2-((3-(2,6-dioxopiperidin-3-yl)-1-methyl-1H-indazol-7-yl)amino)-2-oxoethyl)-2- |
| | (trifluoromethyl)piperazin-1-yl)propoxy)-2-methylphenyl)picolinic acid |
| 247 | 6-[8-(1,3-benzothiazol-2-ylcarbamoyl)-3,4-dihydro-1H-isoquinolin-2-yl]-3-[3-[3-[1- |
| | [[1-[3-(2,6-dioxo-3-piperidyl)-1-methyl-indazol-6-yl]-4-piperidyl]methyl]-4- |
| | piperidyl]propoxy]-2-methyl-phenyl]pyridine-2-carboxylic acid |
| 248 | 6-[8-(1,3-benzothiazol-2-ylcarbamoyl)-3,4-dihydro-1H-isoquinolin-2-yl]-3-[3-[2-[1- |
| | [2-[4-[3-(2,6-dioxo-3-piperidyl)-1-methyl-indazol-6-yl]-1-piperidyl]ethyl]-4- |
| | piperidyl]ethoxy]-2-methyl-phenyl]pyridine-2-carboxylic acid |
| 249 | 6-[8-(1,3-benzothiazol-2-ylcarbamoyl)-3,4-dihydro-1H-isoquinolin-2-yl]-3-[3-[2-[1- |
| | [2-[4-[3-(2,6-dioxo-3-piperidyl)-1-methyl-indazol-7-yl]-1-piperidyl]ethyl]-4- |
| | piperidyl]ethoxy]-2-methyl-phenyl] pyridine-2-carboxylic acid |
| 250 | 6-[8-(1,3-benzothiazol-2-ylcarbamoyl)-3,4-dihydro-1H-isoquinolin-2-yl]-3-[4-[4-[3- |
| | [4-[3-(2,6-dioxo-3-piperidyl)-1-methyl-indazol-7-yl]piperazin-1- |
| | yl]propyl]cyclohexoxy]-2-methyl-phenyl]pyridine-2-carboxylic acid |
| 250a | 6-(8-(benzo[d]thiazol-2-ylcarbamoyl)-3,4-dihydroisoquinolin-2(1H)-yl)-3-(4-(((1s,4r)- |
| | 4-(3-(4-(3-(2,6-dioxopiperidin-3-yl)-1-methyl-1H-indazol-7-yl)piperazin-1- |
| | yl)propyl)cyclohexyl)oxy)-2-methylphenyl)picolinic acid |
| 251 | 6-[8-(1,3-benzothiazol-2-ylcarbamoyl)-3,4-dihydro-1H-isoquinolin-2-yl]-3-[4-[4-[3- |
| | [4-[3-(2,6-dioxo-3-piperidyl)-1-methyl-indazol-6-yl]piperazin-1-yl]-3-oxo- |
| | propyl]cyclohexoxy]-2-methyl-phenyl]pyridine-2-carboxylic acid |
| 251a | 6-(8-(benzo[d]thiazol-2-ylcarbamoyl)-3,4-dihydroisoquinolin-2(1H)-yl)-3-(4-(((1r,4r)- |
| | 4-(3-(4-(3-(2,6-dioxopiperidin-3-yl)-1-methyl-1H-indazol-6-yl)piperazin-1-yl)-3- |
| | oxopropyl)cyclohexyl)oxy)-2-methylphenyl)picolinic acid |
| 252 | 6-[8-(1,3-benzothiazol-2-ylcarbamoyl)-3,4-dihydro-1H-isoquinolin-2-yl]-3-[4-[4-[3- |
| | [4-[3-(2,6-dioxo-3-piperidyl)-1-methyl-indazol-7-yl]piperazin-1-yl]-3-oxo- |
| | propyl]cyclohexoxy]-2-methyl-phenyl]pyridine-2-carboxylic acid |
| 252a | 6-(8-(benzo[d]thiazol-2-ylcarbamoyl)-3,4-dihydroisoquinolin-2(1H)-yl)-3-(4-(((1r,4r)- |
| | 4-(3-(4-(3-(2,6-dioxopiperidin-3-yl)-1-methyl-1H-indazol-7-yl)piperazin-1-yl)-3- |
| | oxopropyl)cyclohexyl)oxy)-2-methylphenyl)picolinic acid |
| 253 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-7-yl]-1-piperidyl]-3-oxo- |
| | propyl]cyclohexoxy]-2-methyl-phenyl]pyridine-2-carboxylic acid |
| 253a | 6-(8-(benzo[d]thiazol-2-ylcarbamoyl)-3,4-dihydroisoquinolin-2(1H)-yl)-3-(3-(((1r,4r)- |
| | 4-(3-(4-(3-(2,6-dioxopiperidin-3-yl)-1-methyl-1H-indazol-7-yl)piperidin-1-yl)-3- |
| | oxopropyl)cyclohexyl)oxy)-2-methylphenyl)picolinic acid |
| 254 | 6-[8-(1,3-benzothiazol-2-ylcarbamoyl)-3,4-dihydro-1H-isoquinolin-2-yl]-3-[4-[4-[3- |
| | [4-[3-(2,6-dioxo-3-piperidyl)-1-methyl-indazol-6-yl]piperazin-1- |
| | yl]propyl]cyclohexoxy]-2-methyl-phenyl]pyridine-2-carboxylic acid |
| 254a | 6-(8-(benzo[d]thiazol-2-ylcarbamoyl)-3,4-dihydroisoquinolin-2(1H)-yl)-3-(4-(((1s,4r)- |
| | 4-(3-(4-(3-(2,6-dioxopiperidin-3-yl)-1-methyl-1H-indazol-6-yl)piperazin-1- |
| | yl)propyl)cyclohexyl)oxy)-2-methylphenyl)picolinic acid |
| 255 | 6-[8-(1,3-benzothiazol-2-ylcarbamoyl)-3,4-dihydro-1H-isoquinolin-2-yl]-3-[3-[3-[1- |
| | [2-[4-[3-(2,6-dioxo-3-piperidyl)-1-methyl-indazol-6-yl]-1-piperidyl]ethyl]-4- |
| | piperidyl]propoxy]-2-methyl-phenyl]pyridine-2-carboxylic acid |
| 256 | 6-[8-(1,3-benzothiazol-2-ylcarbamoyl)-3,4-dihydro-1H-isoquinolin-2-yl]-3-[3-[3-[1- |
| | [2-[4-[3-(2,6-dioxo-3-piperidyl)-1-methyl-indazol-7-yl]-1-piperidyl]ethyl]-4- |
| | piperidyl]propoxy]-2-methyl-phenyl]pyridine-2-carboxylic acid |
| 257 | 6-[8-(1,3-benzothiazol-2-ylcarbamoyl)-3,4-dihydro-1H-isoquinolin-2-yl]-3-[3-[4-[1- |
| | [[1-[3-(2,6-dioxo-3-piperidyl)-1-methyl-indazol-6-yl]-4-piperidyl]methyl]-4- |
| | piperidyl]butoxy]-2-methyl-phenyl]pyridine-2-carboxylic acid |
| 258 | 6-[8-(1,3-benzothiazol-2-ylcarbamoyl)-3,4-dihydro-1H-isoquinolin-2-yl]-3-[3-[2-[1- |
| | [[1-[3-(2,6-dioxo-3-piperidyl)-1-methyl-indazol-6-yl]-4-piperidyl]methyl]-4- |
| | piperidyl]ethoxy]-2-methyl-phenyl]pyridine-2-carboxylic acid |
| 259 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-6-yl]-1-piperidyl]-3-oxo- |
| | propyl]cyclohexoxy]-2-methyl-phenyl]pyridine-2-carboxylic acid |
| 259a | 6-(8-(benzo[d]thiazol-2-ylcarbamoyl)-3,4-dihydroisoquinolin-2(1H)-yl)-3-(3-(((1r,4r)- |
| | 4-(3-(4-(3-(2,6-dioxopiperidin-3-yl)-1-methyl-1H-indazol-6-yl)piperidin-1-yl)-3- |
| | oxopropyl)cyclohexyl)oxy)-2-methylphenyl)picolinic acid |
| 260 | 6-(8-(benzo[d]thiazol-2-ylcarbamoyl)-3,4-dihydroisoquinolin-2(1H)-yl)-3-(3-(3-(4- |
| | ((2-(3-(2,6-dioxopiperidin-3-yl)-1-methyl-1H-indazol-7-yl)-2-azaspiro[3.5]nonan-7- |
| | yl)methyl)piperazin-1-yl)propoxy)-2-methylphenyl)picolinic acid |
| 261 | 6-[8-(1,3-benzothiazol-2-ylcarbamoyl)-3,4-dihydro-1H-isoquinolin-2-yl]-3-[3-[2-[1- |
| | [[1-[3-(2,6-dioxo-3-piperidyl)-1-methyl-indazol-7-yl]-4-piperidyl]methyl]-4- |
| | piperidyl]ethoxy]-2-methyl-phenyl]pyridine-2-carboxylic acid |
| 262 | 6-[8-(1,3-benzothiazol-2-ylcarbamoyl)-3,4-dihydro-1H-isoquinolin-2-yl]-3-[3-[[8-[2- |
| | [[3-(2,6-dioxo-3-piperidyl)-1-methyl-indazol-6-yl]amino]-2-oxo-ethyl]-8- |
| | azaspiro[4.5]decan-3-yl]methoxy]-2-methyl-phenyl]pyridine-2-carboxylic acid |
| 262a | rel-6-(8-(benzo[d]thiazol-2-ylcarbamoyl)-3,4-dihydroisoquinolin-2(1H)-yl)-3-(3- |
| | (((2S)-8-(2-((3-(2,6-dioxopiperidin-3-yl)-1-methyl-1H-indazol-6-yl)amino)-2- |
| | oxoethyl)-8-azaspiro[4.5]decan-2-yl)methoxy)-2-methylphenyl)picolinic acid |
| 262b | rel-6-(8-(benzo[d]thiazol-2-ylcarbamoyl)-3,4-dihydroisoquinolin-2(1H)-yl)-3-(3- |
| | (((2R)-8-(2-((3-(2,6-dioxopiperidin-3-yl)-1-methyl-1H-indazol-6-yl)amino)-2- |
| | oxoethyl)-8-azaspiro[4.5]decan-2-yl)methoxy)-2-methylphenyl)picolinic acid |
| 263 | 6-[8-(1,3-benzothiazol-2-ylcarbamoyl)-3,4-dihydro-1H-isoquinolin-2-yl]-3-[3-[3-[1- |
| | [2-[[3-(2,6-dioxo-3-piperidyl)-1-methyl-indazol-6-yl]amino]-2-oxo-ethyl]-4- |
| | piperidyl]-2-methyl-propoxy]-2-methyl-phenyl]pyridine-2-carboxylic acid |
| 263a | rel-6-(8-(benzo[d]thiazol-2-ylcarbamoyl)-3,4-dihydroisoquinolin-2(1H)-yl)-3-(3- |
| | ((2R)-3-(1-(2-((3-(2,6-dioxopiperidin-3-yl)-1-methyl-1H-indazol-6-yl)amino)-2- |
| | oxoethyl)piperidin-4-yl)-2-methylpropoxy)-2-methylphenyl)picolinic acid |
| 263b | rel-6-(8-(benzo[d]thiazol-2-ylcarbamoyl)-3,4-dihydroisoquinolin-2(1H)-yl)-3-(3- |
| | ((2S)-3-(1-(2-((3-(2,6-dioxopiperidin-3-yl)-1-methyl-1H-indazol-6-yl)amino)-2- |
| | oxoethyl)piperidin-4-yl)-2-methylpropoxy)-2-methylphenyl)picolinic acid |
| 264 | 6-[8-(1,3-benzothiazol-2-ylcarbamoyl)-3,4-dihydro-1H-isoquinolin-2-yl]-3-[4-[3-[1- |
| | [2-[[3-(2,6-dioxo-3-piperidyl)-1-methyl-indazol-6-yl]amino]-2-oxo-ethyl]-4-methyl-4- |
| | piperidyl]propoxy]-2-methyl-phenyl]pyridine-2-carboxylic acid |
| 265 | 6-(8-(benzo[d]thiazol-2-ylcarbamoyl)-3,4-dihydroisoquinolin-2(1H)-yl)-3-(4-((7-(2- |
| | ((3-(2,6-dioxopiperidin-3-yl)-1-methyl-1H-indazol-6-yl)amino)-2-oxoethyl)-7- |
| | azaspiro[3.5]nonan-2-yl)methoxy)-2-methylphenyl)picolinic acid |
| 266 | 6-[8-(1,3-benzothiazol-2-ylcarbamoyl)-3,4-dihydro-1H-isoquinolin-2-yl]-3-[4-[3-[1- |
| | [2-[[3-(2,6-dioxo-3-piperidyl)-1-methyl-indazol-6-yl]amino]-2-oxo-ethyl]-4- |
| | piperidyl]butoxy]-2-methyl-phenyl]pyridine-2-carboxylic acid |
| 266a | rel-6-(8-(benzo[d]thiazol-2-ylcarbamoyl)-3,4-dihydroisoquinolin-2(1H)-yl)-3-(4- |
| | ((3R)-3-(1-(2-((3-(2,6-dioxopiperidin-3-yl)-1-methyl-1H-indazol-6-yl)amino)-2- |
| | oxoethyl)piperidin-4-yl)butoxy)-2-methylphenyl)picolinic acid |
| 266b | rel-6-(8-(benzo[d]thiazol-2-ylcarbamoyl)-3,4-dihydroisoquinolin-2(1H)-yl)-3-(4- |
| | ((3S)-3-(1-(2-((3-(2,6-dioxopiperidin-3-yl)-1-methyl-1H-indazol-6-yl)amino)-2- |
| | oxoethyl)piperidin-4-yl)butoxy)-2-methylphenyl)picolinic acid |
| 267 | 6-[8-(1,3-benzothiazol-2-ylcarbamoyl)-3,4-dihydro-1H-isoquinolin-2-yl]-3-[3-[3-[1- |
| | [2-[[3-(2,6-dioxo-3-piperidyl)-1-methyl-indazol-6-yl]amino]-2-oxo-ethyl]-4-methyl-4- |
| | piperidyl]propoxy]-2-methyl-phenyl]pyridine-2-carboxylic acid |
| 268 | 6-[8-(1,3-benzothiazol-2-ylcarbamoyl)-3,4-dihydro-1H-isoquinolin-2-yl]-3-[3-[8-[2- |
| | [[3-(2,6-dioxo-3-piperidyl)-1-methyl-indazol-6-yl]amino]-2-oxo-ethyl]-8- |
| | azaspiro[4.5]decan-3-yl]oxy]-2-methyl-phenyl]pyridine-2-carboxylic acid |
| 268a | rel-6-(8-(benzo[d]thiazol-2-ylcarbamoyl)-3,4-dihydroisoquinolin-2(1H)-yl)-3-(3- |
| | (((2S)-8-(2-((3-(2,6-dioxopiperidin-3-yl)-1-methyl-1H-indazol-6-yl)amino)-2- |
| | oxoethyl)-8-azaspiro[4.5]decan-2-yl)oxy)-2-methylphenyl)picolinic acid |
| 268b | rel-6-(8-(benzo[d]thiazol-2-ylcarbamoyl)-3,4-dihydroisoquinolin-2(1H)-yl)-3-(3- |
| | (((2R)-8-(2-((3-(2,6-dioxopiperidin-3-yl)-1-methyl-1H-indazol-6-yl)amino)-2- |
| | oxoethyl)-8-azaspiro[4.5]decan-2-yl)oxy)-2-methylphenyl)picolinic acid |
| 269 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-6-yl]-1- |
| | piperidyl]propyl]cyclohexoxy]-2-methyl-phenyl]pyridine-2-carboxylic acid |
| 269a | 6-(8-(benzo[d]thiazol-2-ylcarbamoyl)-3,4-dihydroisoquinolin-2(1H)-yl)-3-(3-(((1s,4r)- |
| | 4-(3-(4-(3-(2,6-dioxopiperidin-3-yl)-1-methyl-1H-indazol-6-yl)piperidin-1- |
| | yl)propyl)cyclohexyl)oxy)-2-methylphenyl)picolinic acid |
| 270 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-7-yl]-1- |
| | piperidyl]propyl]cyclohexoxy]-2-methyl-phenyl]pyridine-2-carboxylic acid |
| 270a | 6-(8-(benzo[d]thiazol-2-ylcarbamoyl)-3,4-dihydroisoquinolin-2(1H)-yl)-3-(3-(((1s,4r)- |
| | 4-(3-(4-(3-(2,6-dioxopiperidin-3-yl)-1-methyl-1H-indazol-7-yl)piperidin-1- |
| | yl)propyl)cyclohexyl)oxy)-2-methylphenyl)picolinic acid |
| 271 | 6-[8-(1,3-benzothiazol-2-ylcarbamoyl)-3,4-dihydro-1H-isoquinolin-2-yl]-3-[4-[3-[1- |
| | [2-[[3-(2,6-dioxo-3-piperidyl)-1-methyl-indazol-6-yl]amino]-2-oxo-ethyl]-4- |
| | piperidyl]-2-methyl-propoxy]-2-methyl-phenyl]pyridine-2-carboxylic acid |
| 271a | rel-6-(8-(benzo[d]thiazol-2-ylcarbamoyl)-3,4-dihydroisoquinolin-2(1H)-yl)-3-(4- |
| | ((2R)-3-(1-(2-((3-(2,6-dioxopiperidin-3-yl)-1-methyl-1H-indazol-6-yl)amino)-2- |
| | oxoethyl)piperidin-4-yl)-2-methylpropoxy)-2-methylphenyl)picolinic acid |
| 271b | rel-6-(8-(benzo[d]thiazol-2-ylcarbamoyl)-3,4-dihydroisoquinolin-2(1H)-yl)-3-(4- |
| | ((2S)-3-(1-(2-((3-(2,6-dioxopiperidin-3-yl)-1-methyl-1H-indazol-6-yl)amino)-2- |
| | oxoethyl)piperidin-4-yl)-2-methylpropoxy)-2-methylphenyl)picolinic acid |
| 272 | 6-[8-(1,3-benzothiazol-2-ylcarbamoyl)-3,4-dihydro-1H-isoquinolin-2-yl]-3-[3-[2-[8- |
| | [2-[[3-(2,6-dioxo-3-piperidyl)-1-methyl-indazol-6-yl]amino]-2-oxo-ethyl]-8- |
| | azaspiro[4.5]decan-3-yl]ethoxy]-2-methyl-phenyl]pyridine-2-carboxylic acid |
| 272a | rel-6-(8-(benzo[d]thiazol-2-ylcarbamoyl)-3,4-dihydroisoquinolin-2(1H)-yl)-3-(3-(2- |
| | ((2S)-8-(2-((3-(2,6-dioxopiperidin-3-yl)-1-methyl-1H-indazol-6-yl)amino)-2- |
| | oxoethyl)-8-azaspiro[4.5]decan-2-yl)ethoxy)-2-methylphenyl)picolinic acid |
| 272b | rel-6-(8-(benzo[d]thiazol-2-ylcarbamoyl)-3,4-dihydroisoquinolin-2(1H)-yl)-3-(3-(2- |
| | ((2R)-8-(2-((3-(2,6-dioxopiperidin-3-yl)-1-methyl-1H-indazol-6-yl)amino)-2- |
| | oxoethyl)-8-azaspiro[4.5]decan-2-yl)ethoxy)-2-methylphenyl)picolinic acid |
| 273 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-6-yl]-2-methyl-piperazin-1- |
| | yl]propyl]cyclohexoxy]-2-methyl-phenyl]pyridine-2-carboxylic acid |
| 273a | 6-(8-(benzo[d]thiazol-2-ylcarbamoyl)-3,4-dihydroisoquinolin-2(1H)-yl)-3-(3- |
| | (((1S,4r)-4-(3-((2R)-4-(3-(2,6-dioxopiperidin-3-yl)-1-methyl-1H-indazol-6-yl)-2- |
| | methylpiperazin-1-yl)propyl)cyclohexyl)oxy)-2-methylphenyl)picolinic acid |
| 273b | 6-(8-(benzo[d]thiazol-2-ylcarbamoyl)-3,4-dihydroisoquinolin-2(1H)-yl)-3-(3- |
| | (((1R,4r)-4-(3-((2S)-4-(3-(2,6-dioxopiperidin-3-yl)-1-methyl-1H-indazol-6-yl)-2- |
| | methylpiperazin-1-yl)propyl)cyclohexyl)oxy)-2-methylphenyl)picolinic acid |
| 274 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-7-yl]-3-methyl-piperazin-1- |
| | yl]propyl]cyclohexoxy]-2-methyl-phenyl]pyridine-2-carboxylic acid |
| 274a | 6-(8-(benzo[d]thiazol-2-ylcarbamoyl)-3,4-dihydroisoquinolin-2(1H)-yl)-3-(3- |
| | (((1S,4r)-4-(3-((3R)-4-(3-(2,6-dioxopiperidin-3-yl)-1-methyl-1H-indazol-7-yl)-3- |
| | methylpiperazin-1-yl)propyl)cyclohexyl)oxy)-2-methylphenyl)picolinic acid |
| 274b | 6-(8-(benzo[d]thiazol-2-ylcarbamoyl)-3,4-dihydroisoquinolin-2(1H)-yl)-3-(3- |
| | (((1R,4r)-4-(3-((3S)-4-(3-(2,6-dioxopiperidin-3-yl)-1-methyl-1H-indazol-7-yl)-3- |
| | methylpiperazin-1-yl)propyl)cyclohexyl)oxy)-2-methylphenyl)picolinic acid |
| 275 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-7-yl]-2-methyl-piperazin-1- |
| | yl]propyl]cyclohexoxy]-2-methyl-phenyl]pyridine-2-carboxylic acid |
| 275a | 6-(8-(benzo[d]thiazol-2-ylcarbamoyl)-3,4-dihydroisoquinolin-2(1H)-yl)-3-(3- |
| | (((1S,4r)-4-(3-((2R)-4-(3-(2,6-dioxopiperidin-3-yl)-1-methyl-1H-indazol-7-yl)-2- |
| | methylpiperazin-1-yl)propyl)cyclohexyl)oxy)-2-methylphenyl)picolinic acid |
| 275b | 6-(8-(benzo[d]thiazol-2-ylcarbamoyl)-3,4-dihydroisoquinolin-2(1H)-yl)-3-(3- |
| | (((1R,4r)-4-(3-((2S)-4-(3-(2,6-dioxopiperidin-3-yl)-1-methyl-1H-indazol-7-yl)-2- |
| | methylpiperazin-1-yl)propyl)cyclohexyl)oxy)-2-methylphenyl)picolinic acid |
| 276 | 6-[8-(1,3-benzothiazol-2-ylcarbamoyl)-3,4-dihydro-1H-isoquinolin-2-yl]-3-[4-[3-[1- |
| | [2-[[3-(2,6-dioxo-3-piperidyl)-1-methyl-indazol-6-yl]amino]-2-oxo-ethyl]-4- |
| | piperidyl]-1-methyl-propoxy]-2-methyl-phenyl]pyridine-2-carboxylic acid |
| 276a | rel-6-(8-(benzo[d]thiazol-2-ylcarbamoyl)-3,4-dihydroisoquinolin-2(1H)-yl)-3-(4- |
| | (((2R)-4-(1-(2-((3-(2,6-dioxopiperidin-3-yl)-1-methyl-1H-indazol-6-yl)amino)-2- |
| | oxoethyl)piperidin-4-yl)butan-2-yl)oxy)-2-methylphenyl)picolinic acid |
| 276b | rel-6-(8-(benzo[d]thiazol-2-ylcarbamoyl)-3,4-dihydroisoquinolin-2(1H)-yl)-3-(4- |
| | (((2S)-4-(1-(2-((3-(2,6-dioxopiperidin-3-yl)-1-methyl-1H-indazol-6-yl)amino)-2- |
| | oxoethyl)piperidin-4-yl)butan-2-yl)oxy)-2-methylphenyl)picolinic acid |
| 277 | 6-(8-(benzo[d]thiazol-2-ylcarbamoyl)-3,4-dihydroisoquinolin-2(1H)-yl)-3-(3-(2-(2-(2- |
| | ((3-(2,6-dioxopiperidin-3-yl)-1-methyl-1H-indazol-6-yl)amino)-2-oxoethyl)-2- |
| | azaspiro[3.3]heptan-6-yl)ethoxy)-2-methylphenyl)picolinic acid |
| 278 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-6-yl]-3-methyl-piperazin-1- |
| | yl]propyl]cyclohexoxy]-2-methyl-phenyl]pyridine-2-carboxylic acid |
| 278a | 6-(8-(benzo[d]thiazol-2-ylcarbamoyl)-3,4-dihydroisoquinolin-2(1H)-yl)-3-(3- |
| | (((1S,4r)-4-(3-((3R)-4-(3-(2,6-dioxopiperidin-3-yl)-1-methyl-1H-indazol-6-yl)-3- |
| | methylpiperazin-1-yl)propyl)cyclohexyl)oxy)-2-methylphenyl)picolinic acid |
| 278b | 6-(8-(benzo[d]thiazol-2-ylcarbamoyl)-3,4-dihydroisoquinolin-2(1H)-yl)-3-(3- |
| | (((1R,4r)-4-(3-((3S)-4-(3-(2,6-dioxopiperidin-3-yl)-1-methyl-1H-indazol-6-yl)-3- |
| | methylpiperazin-1-yl)propyl)cyclohexyl)oxy)-2-methylphenyl)picolinic acid |
| 279 | 6-[8-(1,3-benzothiazol-2-ylcarbamoyl)-3,4-dihydro-1H-isoquinolin-2-yl]-3-[3-[3-[1- |
| | [[1-[3-(2,6-dioxo-3-piperidyl)-1-methyl-indazol-7-yl]-4-piperidyl]methyl]-4- |
| | piperidyl]propoxy]-2-methyl-phenyl]pyridine-2-carboxylic acid |
| 280 | 6-[8-(1,3-benzothiazol-2-ylcarbamoyl)-3,4-dihydro-1H-isoquinolin-2-yl]-3-[3-[3-[6- |
| | [2-[[3-(2,6-dioxo-3-piperidyl)-1-methyl-indazol-6-yl]amino]-2-oxo-ethyl]-3- |
| | pyridyl]propoxy]-2-methyl-phenyl]pyridine-2-carboxylic acid |
| 281 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-6-yl]piperazin-1- |
| | yl]butyl]cyclohexoxy]-2-methyl-phenyl]pyridine-2-carboxylic acid |
| 281a | 6-(8-(benzo[d]thiazol-2-ylcarbamoyl)-3,4-dihydroisoquinolin-2(1H)-yl)-3-(3- |
| | (((1R,4r)-4-((3S*)-3-(4-(3-(2,6-dioxopiperidin-3-yl)-1-methyl-1H-indazol-6- |
| | yl)piperazin-1-yl)butyl)cyclohexyl)oxy)-2-methylphenyl)picolinic acid |
| 281b | 6-(8-(benzo[d]thiazol-2-ylcarbamoyl)-3,4-dihydroisoquinolin-2(1H)-yl)-3-(3- |
| | (((1S,4r)-4-((3R*)-3-(4-(3-(2,6-dioxopiperidin-3-yl)-1-methyl-1H-indazol-6- |
| | yl)piperazin-1-yl)butyl)cyclohexyl)oxy)-2-methylphenyl)picolinic acid |
| 282 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-6-yl]piperazin-1-yl]-2-methyl- |
| | propyl]cyclohexoxy]-2-methyl-phenyl]pyridine-2-carboxylic acid |
| 282a | 6-(8-(benzo[d]thiazol-2-ylcarbamoyl)-3,4-dihydroisoquinolin-2(1H)-yl)-3-(3- |
| | (((1R,4r)-4-((2R*)-3-(4-(3-(2,6-dioxopiperidin-3-yl)-1-methyl-1H-indazol-6- |
| | yl)piperazin-1-yl)-2-methylpropyl)cyclohexyl)oxy)-2-methylphenyl)picolinic acid |
| 282b | 6-(8-(benzo[d]thiazol-2-ylcarbamoyl)-3,4-dihydroisoquinolin-2(1H)-yl)-3-(3- |
| | (((1S,4r)-4-((2S*)-3-(4-(3-(2,6-dioxopiperidin-3-yl)-1-methyl-1H-indazol-6- |
| | yl)piperazin-1-yl)-2-methylpropyl)cyclohexyl)oxy)-2-methylphenyl)picolinic acid |
| 283 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-7-yl]piperazin-1- |
| | yl]butyl]cyclohexoxy]-2-methyl-phenyl]pyridine-2-carboxylic acid |
| 283a | 6-(8-(benzo[d]thiazol-2-ylcarbamoyl)-3,4-dihydroisoquinolin-2(1H)-yl)-3-(3- |
| | (((1R,4r)-4-((3S*)-3-(4-(3-(2,6-dioxopiperidin-3-yl)-1-methyl-1H-indazol-7- |
| | yl)piperazin-1-yl)butyl)cyclohexyl)oxy)-2-methylphenyl)picolinic acid |
| 283b | 6-(8-(benzo[d]thiazol-2-ylcarbamoyl)-3,4-dihydroisoquinolin-2(1H)-yl)-3-(3- |
| | (((1S,4r)-4-((3R*)-3-(4-(3-(2,6-dioxopiperidin-3-yl)-1-methyl-1H-indazol-7- |
| | yl)piperazin-1-yl)butyl)cyclohexyl)oxy)-2-methylphenyl)picolinic acid |
| 284 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-7-yl]piperazin-1-yl]-2-methyl- |
| | propyl]cyclohexoxy]-2-methyl-phenyl]pyridine-2-carboxylic acid |
| 284a | 6-(8-(benzo[d]thiazol-2-ylcarbamoyl)-3,4-dihydroisoquinolin-2(1H)-yl)-3-(3- |
| | (((1S,4r)-4-((2S*)-3-(4-(3-(2,6-dioxopiperidin-3-yl)-1-methyl-1H-indazol-7- |
| | yl)piperazin-1-yl)-2-methylpropyl)cyclohexyl)oxy)-2-methylphenyl)picolinic acid |
| 284b | 6-(8-(benzo[d]thiazol-2-ylcarbamoyl)-3,4-dihydroisoquinolin-2(1H)-yl)-3-(3- |
| | (((1R,4r)-4-((2R*)-3-(4-(3-(2,6-dioxopiperidin-3-yl)-1-methyl-1H-indazol-7- |
| | yl)piperazin-1-yl)-2-methylpropyl)cyclohexyl)oxy)-2-methylphenyl)picolinic acid |
| 285 | 6-[8-(1,3-benzothiazol-2-ylcarbamoyl)-3,4-dihydro-1H-isoquinolin-2-yl]-3-[3-[4-[1- |
| | [[1-[3-(2,6-dioxo-3-piperidyl)-1-methyl-indazol-7-yl]-4-piperidyl]methyl]-4- |
| | piperidyl]butoxy]-2-methyl-phenyl]pyridine-2-carboxylic acid |
| 286 | 6-[8-(1,3-benzothiazol-2-ylcarbamoyl)-3,4-dihydro-1H-isoquinolin-2-yl]-3-[3-[3-[2- |
| | [2-[[3-(2,6-dioxo-3-piperidyl)-1-methyl-indazol-6-yl]amino]-2-oxo-ethyl]-2- |
| | azaspiro[3.3]heptan-6-yl]propoxy]-2-methyl-phenyl]pyridine-2-carboxylic acid |
| 287 | 6-[8-(1,3-benzothiazol-2-ylcarbamoyl)-3,4-dihydro-1H-isoquinolin-2-yl]-3-[3-[2-[1- |
| | [3-[1-[3-(2,6-dioxo-3-piperidyl)-1-methyl-indazol-6-yl]-4-piperidyl]propyl]-4- |
| | piperidyl]ethoxy]-2-methyl-phenyl]pyridine-2-carboxylic acid |
| 288 | 6-[8-(1,3-benzothiazol-2-ylcarbamoyl)-3,4-dihydro-1H-isoquinolin-2-yl]-3-[3-[2-[1- |
| | [3-[1-[3-(2,6-dioxo-3-piperidyl)-1-methyl-indazol-7-yl]-4-piperidyl]propyl]-4- |
| | piperidyl]ethoxyl-2-methyl-phenyl]pyridine-2-carboxylic acid |
| 289 | 6-[8-(1,3-benzothiazol-2-ylcarbamoyl)-3,4-dihydro-1H-isoquinolin-2-yl]-3-[3-[2-[6- |
| | [2-[[3-(2,6-dioxo-3-piperidyl)-1-methyl-indazol-6-yl]amino]-2-oxo-ethyl]-6- |
| | azaspiro[2.5]octan-2-yl]ethoxy]-2-methyl-phenyl]pyridine-2-carboxylic acid |
| 289a | rel-6-(8-(benzo[d]thiazol-2-ylcarbamoyl)-3,4-dihydroisoquinolin-2(1H)-yl)-3-(3-(2- |
| | ((1S)-6-(2-((3-(2,6-dioxopiperidin-3-yl)-1-methyl-1H-indazol-6-yl)amino)-2- |
| | oxoethyl)-6-azaspiro[2.5]octan-1-yl)ethoxy)-2-methylphenyl)picolinic acid |
| 289b | rel-6-(8-(benzo[d]thiazol-2-ylcarbamoyl)-3,4-dihydroisoquinolin-2(1H)-yl)-3-(3-(2- |
| | ((1R)-6-(2-((3-(2,6-dioxopiperidin-3-yl)-1-methyl-1H-indazol-6-yl)amino)-2- |
| | oxoethyl)-6-azaspiro[2.5]octan-1-yl)ethoxy)-2-methylphenyl)picolinic acid |
| 290 | 6-[8-(1,3-benzothiazol-2-ylcarbamoyl)-3,4-dihydro-1H-isoquinolin-2-yl]-3-[3-[3-[[1- |
| | [2-[[3-(2,6-dioxo-3-piperidyl)-1-methyl-indazol-6-yl]amino]-2-oxo-ethyl]-4- |
| | piperidyl]oxy]propyl]-2-methyl-phenyl]pyridine-2-carboxylic acid |
| 291 | 6-[8-(1,3-benzothiazol-2-ylcarbamoyl)-3,4-dihydro-1H-isoquinolin-2-yl]-3-[4-[3-[[1- |
| | [2-[[3-(2,6-dioxo-3-piperidyl)-1-methyl-indazol-6-yl]amino]-2-oxo-ethyl]-4- |
| | piperidyl]oxy]propyl]-2-methyl-phenyl]pyridine-2-carboxylic acid |
| 292 | 6-[8-(1,3-benzothiazol-2-ylcarbamoyl)-3,4-dihydro-1H-isoquinolin-2-yl]-3-[3-[4-[1- |
| | [2-[[3-(2,6-dioxo-3-piperidyl)-1-methyl-indazol-6-yl]amino]-2-oxo-ethyl]-4- |
| | piperidyl]butyl]-2-methyl-phenyl]pyridine-2-carboxylic acid |
| 293 | 6-[8-(1,3-benzothiazol-2-ylcarbamoyl)-3,4-dihydro-1H-isoquinolin-2-yl]-3-[3-[4-[4- |
| | [4-[3-(2,6-dioxo-3-piperidyl)-7-fluoro-1-methyl-indazol-6-yl]piperazin-1- |
| | yl]butyl]cyclohexoxy]-2-methyl-phenyl]pyridine-2-carboxylic acid |
| 293a | 6-(8-(benzo[d]thiazol-2-ylcarbamoyl)-3,4-dihydroisoquinolin-2(1H)-yl)-3-(3-(((1s,4r)- |
| | 4-(4-(4-(3-(2,6-dioxopiperidin-3-yl)-7-fluoro-1-methyl-1H-indazol-6-yl)piperazin-1- |
| | yl)butyl)cyclohexyl)oxy)-2-methylphenyl)picolinic acid |
| 294 | 6-[8-(1,3-benzothiazol-2-ylcarbamoyl)-3,4-dihydro-1H-isoquinolin-2-yl]-3-[4-[4-[1- |
| | [2-[[3-(2,6-dioxo-3-piperidyl)-1-methyl-indazol-6-yl]amino]-2-oxo-ethyl]-4- |
| | piperidyl]butyl]-2-methyl-phenyl]pyridine-2-carboxylic acid |
| 295 | 6-[8-(1,3-benzothiazol-2-ylcarbamoyl)-3,4-dihydro-1H-isoquinolin-2-yl]-3-[3-[3-[1- |
| | [2-[[3-(2,6-dioxo-3-piperidyl)-5-fluoro-1-methyl-indazol-6-yl]amino]-2-oxo-ethyl]-4- |
| | piperidyl]propoxy]-2-methyl-phenyl]pyridine-2-carboxylic acid |
| 296 | 6-[8-(1,3-benzothiazol-2-ylcarbamoyl)-3,4-dihydro-1H-isoquinolin-2-yl]-3-[3-[4-[4- |
| | [4-[3-(2,6-dioxo-3-piperidyl)-5-fluoro-1-methyl-indazol-6-yl]piperazin-1- |
| | yl]butyl]cyclohexoxy]-2-methyl-phenyl]pyridine-2-carboxylic acid |
| 296a | 6-(8-(benzo[d]thiazol-2-ylcarbamoyl)-3,4-dihydroisoquinolin-2(1H)-yl)-3-(3-(((1s,4r)- |
| | 4-(4-(4-(3-(2,6-dioxopiperidin-3-yl)-5-fluoro-1-methyl-1H-indazol-6-yl)piperazin-1- |
| | yl)butyl)cyclohexyl)oxy)-2-methylphenyl)picolinic acid |
| 297 | 6-[8-(1,3-benzothiazol-2-ylcarbamoyl)-3,4-dihydro-1H-isoquinolin-2-yl]-3-[3-[4-[4- |
| | [4-[3-(2,6-dioxo-3-piperidyl)-1-methyl-indazol-6-yl]-2-methyl-piperazin-1- |
| | yl]butyl]cyclohexoxy]-2-methyl-phenyl]pyridine-2-carboxylic acid |
| 297a | 6-(8-(benzo[d]thiazol-2-ylcarbamoyl)-3,4-dihydroisoquinolin-2(1H)-yl)-3-(3-(((1s,4r)- |
| | 4-(4-((2R)-4-(3-(2,6-dioxopiperidin-3-yl)-1-methyl-1H-indazol-6-yl)-2- |
| | methylpiperazin-1-yl)butyl)cyclohexyl)oxy)-2-methylphenyl)picolinic acid |
| 297b | 6-(8-(benzo[d]thiazol-2-ylcarbamoyl)-3,4-dihydroisoquinolin-2(1H)-yl)-3-(3-(((1r,4r)- |
| | 4-(4-((2S)-4-(3-(2,6-dioxopiperidin-3-yl)-1-methyl-1H-indazol-6-yl)-2- |
| | methylpiperazin-1-yl)butyl)cyclohexyl)oxy)-2-methylphenyl)picolinic acid |
| 298 | 6-[8-(1,3-benzothiazol-2-ylcarbamoyl)-3,4-dihydro-1H-isoquinolin-2-yl]-3-[3-[4-[4- |
| | [4-[3-(2,6-dioxo-3-piperidyl)-1-methyl-indazol-7-yl]-1-piperidyl]butyl]cyclohexoxy]- |
| | 2-methyl-phenyl]pyridine-2-carboxylic acid |
| 298a | 6-(8-(benzo[d]thiazol-2-ylcarbamoyl)-3,4-dihydroisoquinolin-2(1H)-yl)-3-(3-(((1s,4r)- |
| | 4-(4-(4-(3-(2,6-dioxopiperidin-3-yl)-1-methyl-1H-indazol-7-yl)piperidin-1- |
| | yl)butyl)cyclohexyl)oxy)-2-methylphenyl)picolinic acid |
| 299 | 6-[8-(1,3-benzothiazol-2-ylcarbamoyl)-3,4-dihydro-1H-isoquinolin-2-yl]-3-[3-[4-[4- |
| | [4-[3-(2,6-dioxo-3-piperidyl)-1-methyl-indazol-6-yl]-1-piperidyl]butyl]cyclohexoxy]- |
| | 2-methyl-phenyl]pyridine-2-carboxylic acid |
| 299a | 6-(8-(benzo[d]thiazol-2-ylcarbamoyl)-3,4-dihydroisoquinolin-2(1H)-yl)-3-(3-(((1s,4r)- |
| | 4-(4-(4-(3-(2,6-dioxopiperidin-3-yl)-1-methyl-1H-indazol-6-yl)piperidin-1- |
| | yl)butyl)cyclohexyl)oxy)-2-methylphenyl)picolinic acid |
| 300 | 6-[8-(1,3-benzothiazol-2-ylcarbamoyl)-3,4-dihydro-1H-isoquinolin-2-yl]-3-[3-[4-[4- |
| | [4-[3-(2,6-dioxo-3-piperidyl)-1-methyl-indazol-6-yl]-3-methyl-piperazin-1- |
| | yl]butyl]cyclohexoxy]-2-methyl-phenyl]pyridine-2-carboxylic acid |
| 300a | 6-(8-(benzo[d]thiazol-2-ylcarbamoyl)-3,4-dihydroisoquinolin-2(1H)-yl)-3-(3-(((1s,4r)- |
| | 4-(4-((3R)-4-(3-(2,6-dioxopiperidin-3-yl)-1-methyl-1H-indazol-6-yl)-3- |
| | methylpiperazin-1-yl)butyl)cyclohexyl)oxy)-2-methylphenyl)picolinic acid |
| 300b | 6-(8-(benzo[d]thiazol-2-ylcarbamoyl)-3,4-dihydroisoquinolin-2(1H)-yl)-3-(3-(((1r,4r)- |
| | 4-(4-((3S)-4-(3-(2,6-dioxopiperidin-3-yl)-1-methyl-1H-indazol-6-yl)-3- |
| | methylpiperazin-1-yl)butyl)cyclohexyl)oxy)-2-methylphenyl)picolinic acid |
| 301 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-6-yl]-3-(trifluoromethyl)piperazin-1- |
| | yl]propyl]cyclohexoxy]-2-methyl-phenyl]pyridine-2-carboxylic acid |
| 301a | 6-(8-(benzo[d]thiazol-2-ylcarbamoyl)-3,4-dihydroisoquinolin-2(1H)-yl)-3-(3-(((1s,4r)- |
| | 4-(3-((3R)-4-(3-(2,6-dioxopiperidin-3-yl)-1-methyl-1H-indazol-6-yl)-3- |
| | (trifluoromethyl)piperazin-1-yl)propyl)cyclohexyl)oxy)-2-methylphenyl)picolinic acid |
| 301b | 6-(8-(benzo[d]thiazol-2-ylcarbamoyl)-3,4-dihydroisoquinolin-2(1H)-yl)-3-(3-(((1r,4r)- |
| | 4-(3-((3S)-4-(3-(2,6-dioxopiperidin-3-yl)-1-methyl-1H-indazol-6-yl)-3- |
| | (trifluoromethyl)piperazin-1-yl)propyl)cyclohexyl)oxy)-2-methylphenyl)picolinic acid |
| 302 | 6-[8-(1,3-benzothiazol-2-ylcarbamoyl)-3,4-dihydro-1H-isoquinolin-2-yl]-3-[3-[3-[1- |
| | [2-[[3-(2,6-dioxo-3-piperidyl)-7-fluoro-1-methyl-indazol-6-yl]amino]-2-oxo-ethyl]-4- |
| | piperidyl]propoxy]-2-methyl-phenyl]pyridine-2-carboxylic acid |
| 303 | 6-(8-(benzo[d]thiazol-2-ylcarbamoyl)-3,4-dihydroisoquinolin-2(1H)-yl)-3-(3-(3-(1-(2- |
| | (3-(2,6-dioxopiperidin-3-yl)-1-methyl-1H-indazol-6-yl)ethyl)piperidin-4-yl)propoxy)- |
| | 2-methylphenyl)picolinic acid |
| 304 | 6-(8-(benzo[d]thiazol-2-ylcarbamoyl)-3,4-dihydroisoquinolin-2(1H)-yl)-3-(3-(3-(1- |
| | ((3-(2,6-dioxopiperidin-3-yl)-1-methyl-1H-indazol-6-yl)methyl)piperidin-4- |
| | yl)propoxy)-2-methylphenyl)picolinic acid |
| 305 | 6-(8-(benzo[d]thiazol-2-ylcarbamoyl)-3,4-dihydroisoquinolin-2(1H)-yl)-3-(3-(3-(1-(3- |
| | (3-(2,6-dioxopiperidin-3-yl)-1-methyl-1H-indazol-6-yl)propyl)piperidin-4- |
| | yl)propoxy)-2-methylphenyl)picolinic acid |
| 306 | 6-(8-(benzo[d]thiazol-2-ylcarbamoyl)-3,4-dihydroisoquinolin-2(1H)-yl)-3-(3-(3-(1'-(3- |
| | (2,6-dioxopiperidin-3-yl)-1-methyl-1H-indazol-6-yl)-[1,4'-bipiperidin]-4-yl)propoxy)- |
| | 2-methylphenyl)picolinic acid |
| 307 | 6-[8-(1,3-benzothiazol-2-ylcarbamoyl)-3,4-dihydro-1H-isoquinolin-2-yl]-3-[3-[4-[4- |
| | [4-[3-(2,6-dioxo-3-piperidyl)-1-methyl-indazol-6-yl]piperazin-1-yl]-2-methyl- |
| | butyl]cyclohexoxy]-2-methyl-phenyl]pyridine-2-carboxylic acid |
| 307a | 6-(8-(benzo[d]thiazol-2-ylcarbamoyl)-3,4-dihydroisoquinolin-2(1H)-yl)-3-(3- |
| | (((1S,4r)-4-((2S*)-4-(4-(3-(2,6-dioxopiperidin-3-yl)-1-methyl-1H-indazol-6- |
| | yl)piperazin-1-yl)-2-methylbutyl)cyclohexyl)oxy)-2-methylphenyl)picolinic acid |
| 307b | 6-(8-(benzo[d]thiazol-2-ylcarbamoyl)-3,4-dihydroisoquinolin-2(1H)-yl)-3-(3- |
| | (((1R,4r)-4-((2R*)-4-(4-(3-(2,6-dioxopiperidin-3-yl)-1-methyl-1H-indazol-6- |
| | yl)piperazin-1-yl)-2-methylbutyl)cyclohexyl)oxy)-2-methylphenyl)picolinic acid |
| 308 | 6-[8-(1,3-benzothiazol-2-ylcarbamoyl)-3,4-dihydro-1H-isoquinolin-2-yl]-3-[3-[4-[4- |
| | [4-[3-(2,6-dioxo-3-piperidyl)-1-methyl-indazol-6-yl]piperazin-1-yl]-3-methyl- |
| | butyl]cyclohexoxy]-2-methyl-phenyl]pyridine-2-carboxylic acid |
| 308a | 6-(8-(benzo[d]thiazol-2-ylcarbamoyl)-3,4-dihydroisoquinolin-2(1H)-yl)-3-(3- |
| | (((1S,4r)-4-((3R*)-4-(4-(3-(2,6-dioxopiperidin-3-yl)-1-methyl-1H-indazol-6- |
| | yl)piperazin-1-yl)-3-methylbutyl)cyclohexyl)oxy)-2-methylphenyl)picolinic acid |
| 308b | 6-(8-(benzo[d]thiazol-2-ylcarbamoyl)-3,4-dihydroisoquinolin-2(1H)-yl)-3-(3- |
| | (((1R,4r)-4-((3S*)-4-(4-(3-(2,6-dioxopiperidin-3-yl)-1-methyl-1H-indazol-6- |
| | yl)piperazin-1-yl)-3-methylbutyl)cyclohexyl)oxy)-2-methylphenyl)picolinic acid |
| 309 | 6-[8-(1,3-benzothiazol-2-ylcarbamoyl)-3,4-dihydro-1H-isoquinolin-2-yl]-3-[3-[3-[5- |
| | [2-[[3-(2,6-dioxo-3-piperidyl)-1-methyl-indazol-6-yl]amino]-2-oxo-ethyl]-2- |
| | pyridyl]propoxy]-2-methyl-phenyl]pyridine-2-carboxylic acid |
| 310 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-6-yl]piperazin-1-yl]-1-methyl- |
| | propyl]cyclohexoxy]-2-methyl-phenyl]pyridine-2-carboxylic acid |
| 310a | 6-(8-(benzo[d]thiazol-2-ylcarbamoyl)-3,4-dihydroisoquinolin-2(1H)-yl)-3-(3- |
| | (((1R,4r)-4-((2R*)-4-(4-(3-(2,6-dioxopiperidin-3-yl)-1-methyl-1H-indazol-6- |
| | yl)piperazin-1-yl)butan-2-yl)cyclohexyl)oxy)-2-methylphenyl)picolinic acid |
| 310b | 6-(8-(benzo[d]thiazol-2-ylcarbamoyl)-3,4-dihydroisoquinolin-2(1H)-yl)-3-(3- |
| | (((1S,4r)-4-((2S*)-4-(4-(3-(2,6-dioxopiperidin-3-yl)-1-methyl-1H-indazol-6- |
| | yl)piperazin-1-yl)butan-2-yl)cyclohexyl)oxy)-2-methylphenyl)picolinic acid |
| 311 | 6-[8-(1,3-benzothiazol-2-ylcarbamoyl)-3,4-dihydro-1H-isoquinolin-2-yl]-3-[3-[4-[3- |
| | [1-[3-(2,6-dioxo-3-piperidyl)-1-methyl-indazol-6-yl]-4- |
| | piperidyl]propyl]cyclohexoxy]-2-methyl-phenyl]pyridine-2-carboxylic acid |
| 311a | 6-(8-(benzo[d]thiazol-2-ylcarbamoyl)-3,4-dihydroisoquinolin-2(1H)-yl)-3-(3-(((1r,4s)- |
| | 4-(3-(1-(3-(2,6-dioxopiperidin-3-yl)-1-methyl-1H-indazol-6-yl)piperidin-4- |
| | yl)propyl)cyclohexyl)oxy)-2-methylphenyl) picolinic acid |
| 311b | 6-(8-(benzo[d]thiazol-2-ylcarbamoyl)-3,4-dihydroisoquinolin-2(1H)-yl)-3-(3-(((1s,4r)- |
| | 4-(3-(1-(3-(2,6-dioxopiperidin-3-yl)-1-methyl-1H-indazol-6-yl)piperidin-4- |
| | yl)propyl)cyclohexyl)oxy)-2-methylphenyl)picolinic acid |
| 312 | 6-(8-(benzo[d]thiazol-2-ylcarbamoyl)-3,4-dihydroisoquinolin-2(1H)-yl)-3-(3-(3-(1- |
| | ((1-(3-(2,6-dioxopiperidin-3-yl)-1-methyl-1H-indazol-6-yl)azetidin-3- |
| | yl)methyl)piperidin-4-yl)propoxy)-2-methylphenyl)picolinic acid |
| 313 | 6-[8-(1,3-benzothiazol-2-ylcarbamoyl)-3,4-dihydro-1H-isoquinolin-2-yl]-3-[3-[3-[1- |
| | [2-[[3-(2,6-dioxo-3-piperidyl)-1-methyl-indazol-6-yl]amino]-2-oxo-ethyl]-4- |
| | piperidyl]propoxy]-2-(trifluoromethyl)phenyl]pyridine-2-carboxylic acid |
| 314 | 6-(8-(benzo[d]thiazol-2-ylcarbamoyl)-3,4-dihydroisoquinolin-2(1H)-yl)-3-(3-(3-(1-(2- |
| | ((3-(2,6-dioxopiperidin-3-yl)-1-methyl-1H-indazol-6-yl)amino)-2-oxoethyl)piperidin- |
| | 4-yl)propoxy)phenyl)picolinic acid |
| 316 | 6-[8-(1,3-benzothiazol-2-ylcarbamoyl)-3,4-dihydro-1H-isoquinolin-2-yl]-3-[3-[4-[3- |
| | [4-[3-(2,4-dioxohexahydropyrimidin-1-yl)-1-methyl-indazol-6-yl]piperazin-1- |
| | yl]propyl]cyclohexoxy]-2-methyl-phenyl]pyridine-2-carboxylic acid |
| 316a | 6-(8-(benzo[d]thiazol-2-ylcarbamoyl)-3,4-dihydroisoquinolin-2(1H)-yl)-3-(3-(((1s,4r)- |
| | 4-(3-(4-(3-(2,4-dioxotetrahydropyrimidin-1(2H)-yl)-1-methyl-1H-indazol-6- |
| | yl)piperazin-1-yl)propyl)cyclohexyl)oxy)-2-methylphenyl)picolinic acid |
| 317 | 6-(8-(benzo[d]thiazol-2-ylcarbamoyl)-3,4-dihydroisoquinolin-2(1H)-yl)-3-(3-(3-(1-(2- |
| | ((3-(2,4-dioxotetrahydropyrimidin-1(2H)-yl)-1-methyl-1H-indazol-6-yl)amino)-2- |
| | oxoethyl)piperidin-4-yl)propoxy)-2-methylphenyl)picolinic acid |
| 318 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-6-yl]-3,3-dimethyl-piperazin-1- |
| | yl]propyl]cyclohexoxy]-2-methyl-phenyl]pyridine-2-carboxylic acid |
| 318a | 6-(8-(benzo[d]thiazol-2-ylcarbamoyl)-3,4-dihydroisoquinolin-2(1H)-yl)-3-(3-(((1s,4r)- |
| | 4-(3-(4-(3-(2,6-dioxopiperidin-3-yl)-1-methyl-1H-indazol-6-yl)-3,3- |
| | dimethylpiperazin-1-yl)propyl)cyclohexyl)oxy)-2-methylphenyl)picolinic acid |
| 319 | 6-[8-(1,3-benzothiazol-2-ylcarbamoyl)-3,4-dihydro-1H-isoquinolin-2-yl]-3-[3-[4-[4- |
| | [4-[3-(2,6-dioxo-3-piperidyl)-1-methyl-indazol-6-yl]piperazin-1- |
| | yl]butyl]cyclohexoxy]-2-(trifluoromethyl)phenyl]pyridine-2-carboxylic acid |
| 319a | 6-(8-(benzo[d]thiazol-2-ylcarbamoyl)-3,4-dihydroisoquinolin-2(1H)-yl)-3-(3-(((1s,4r)- |
| | 4-(4-(4-(3-(2,6-dioxopiperidin-3-yl)-1-methyl-1H-indazol-6-yl)piperazin-1- |
| | yl)butyl)cyclohexyl)oxy)-2-(trifluoromethyl)phenyl)picolinic acid |
| 320 | 6-[8-(1,3-benzothiazol-2-ylcarbamoyl)-3,4-dihydro-1H-isoquinolin-2-yl]-3-[3-[4-[3- |
| | [3-[3-(2,6-dioxo-3-piperidyl)-1-methyl-indazol-7-yl]pyrrolidin-1- |
| | yl]propyl]cyclohexoxy]-2-methyl-phenyl]pyridine-2-carboxylic acid |
| 320a | 6-(8-(benzo[d]thiazol-2-ylcarbamoyl)-3,4-dihydroisoquinolin-2(1H)-yl)-3-(3- |
| | (((1S,4r)-4-(3-((3R*)-3-(3-(2,6-dioxopiperidin-3-yl)-1-methyl-1H-indazol-7- |
| | yl)pyrrolidin-1-yl)propyl)cyclohexyl)oxy)-2-methylphenyl)picolinic acid |
| 320b | 6-(8-(benzo[d]thiazol-2-ylcarbamoyl)-3,4-dihydroisoquinolin-2(1H)-yl)-3-(3- |
| | (((1R,4r)-4-(3-((3S*)-3-(3-(2,6-dioxopiperidin-3-yl)-1-methyl-1H-indazol-7- |
| | yl)pyrrolidin-1-yl)propyl)cyclohexyl)oxy)-2-methylphenyl)picolinic acid |
| 321 | 6-[8-(1,3-benzothiazol-2-ylcarbamoyl)-3,4-dihydro-1H-isoquinolin-2-yl]-3-[3-[4-[4- |
| | [3-[3-(2,6-dioxo-3-piperidyl)-1-methyl-indazol-7-yl]pyrrolidin-1- |
| | yl]butyl]cyclohexoxy]-2-methyl-phenyl]pyridine-2-carboxylic acid |
| 321a | 6-(8-(benzo[d]thiazol-2-ylcarbamoyl)-3,4-dihydroisoquinolin-2(1H)-yl)-3-(3- |
| | (((1S,4r)-4-(4-((3R*)-3-(3-(2,6-dioxopiperidin-3-yl)-1-methyl-1H-indazol-7- |
| | yl)pyrrolidin-1-yl)butyl)cyclohexyl)oxy)-2-methylphenyl)picolinic acid |
| 321b | 6-(8-(benzo[d]thiazol-2-ylcarbamoyl)-3,4-dihydroisoquinolin-2(1H)-yl)-3-(3- |
| | (((1R,4r)-4-(4-((3S*)-3-(3-(2,6-dioxopiperidin-3-yl)-1-methyl-1H-indazol-7- |
| | yl)pyrrolidin-1-yl)butyl)cyclohexyl)oxy)-2-methylphenyl)picolinic acid |
| 322 | 6-[8-(1,3-benzothiazol-2-ylcarbamoyl)-3,4-dihydro-1H-isoquinolin-2-yl]-3-[3-[4-[4- |
| | [4-[3-(2,6-dioxo-3-piperidyl)-1-methyl-indazol-6-yl]-3,3-dimethyl-piperazin-1- |
| | yl]butyl]cyclohexoxy]-2-methyl-phenyl]pyridine-2-carboxylic acid |
| 322a | 6-(8-(benzo[d]thiazol-2-ylcarbamoyl)-3,4-dihydroisoquinolin-2(1H)-yl)-3-(3-(((1s,4r)- |
| | 4-(4-(4-(3-(2,6-dioxopiperidin-3-yl)-1-methyl-1H-indazol-6-yl)-3,3- |
| | dimethylpiperazin-1-yl)butyl)cyclohexyl)oxy)-2-methylphenyl)picolinic acid |
| 323 | 6-[8-(1,3-benzothiazol-2-ylcarbamoyl)-3,4-dihydro-1H-isoquinolin-2-yl]-3-[3-[4-[4- |
| | [4-[3-(2,6-dioxo-3-piperidyl)-1-methyl-indazol-6-yl]-2,2-dimethyl-piperazin-1- |
| | yl]butyl]cyclohexoxy]-2-methyl-phenyl]pyridine-2-carboxylic acid |
| 323a | 6-(8-(benzo[d]thiazol-2-ylcarbamoyl)-3,4-dihydroisoquinolin-2(1H)-yl)-3-(3-(((1s,4r)- |
| | 4-(4-(4-(3-(2,6-dioxopiperidin-3-yl)-1-methyl-1H-indazol-6-yl)-2,2- |
| | dimethylpiperazin-1-yl)butyl)cyclohexyl)oxy)-2-methylphenyl)picolinic acid |
| 324 | 6-[8-(1,3-benzothiazol-2-ylcarbamoyl)-3,4-dihydro-1H-isoquinolin-2-yl]-3-[3-[4-[4- |
| | [4-[3-(2,6-dioxo-3-piperidyl)-1,7-dimethyl-indazol-6-yl]piperazin-1- |
| | yl]butyl]cyclohexoxy]-2-methyl-phenyl]pyridine-2-carboxylic acid |
| 324a | 6-(8-(benzo[d]thiazol-2-ylcarbamoyl)-3,4-dihydroisoquinolin-2(1H)-yl)-3-(3-(((1s,4r)- |
| | 4-(4-(4-(3-(2,6-dioxopiperidin-3-yl)-1,7-dimethyl-1H-indazol-6-yl)piperazin-1- |
| | yl)butyl)cyclohexyl)oxy)-2-methylphenyl)picolinic acid |
| 325 | 6-[8-(1,3-benzothiazol-2-ylcarbamoyl)-3,4-dihydro-1H-isoquinolin-2-yl]-3-[3-[4-[4- |
| | [4-[3-(2,6-dioxo-3-piperidyl)-1,5-dimethyl-indazol-6-yl]piperazin-1- |
| | yl]butyl]cyclohexoxy]-2-methyl-phenyl]pyridine-2-carboxylic acid |
| 325a | 6-(8-(benzo[d]thiazol-2-ylcarbamoyl)-3,4-dihydroisoquinolin-2(1H)-yl)-3-(3-(((1s,4r)- |
| | 4-(4-(4-(3-(2,6-dioxopiperidin-3-yl)-1,5-dimethyl-1H-indazol-6-yl)piperazin-1- |
| | yl)butyl)cyclohexyl)oxy)-2-methylphenyl)picolinic acid |
| 326 | 6-[8-(1,3-benzothiazol-2-ylcarbamoyl)-3,4-dihydro-1H-isoquinolin-2-yl]-3-[3-[4-[4- |
| | [4-[3-(2,6-dioxo-3-piperidyl)-1-methyl-indazol-6-yl]oxy-1- |
| | piperidyl]butyl]cyclohexoxy]-2-methyl-phenyl]pyridine-2-carboxylic acid |
| 326a | 6-(8-(benzo[d]thiazol-2-ylcarbamoyl)-3,4-dihydroisoquinolin-2(1H)-yl)-3-(3-(((1s,4r)- |
| | 4-(4-(4-((3-(2,6-dioxopiperidin-3-yl)-1-methyl-1H-indazol-6-yl)oxy)piperidin-1- |
| | yl)butyl)cyclohexyl)oxy)-2-methylphenyl)picolinic acid |
| 327 | 6-[8-(1,3-benzothiazol-2-ylcarbamoyl)-3,4-dihydro-1H-isoquinolin-2-yl]-3-[3-[4-[4- |
| | [4-[3-(2,6-dioxo-3-piperidyl)-1-methyl-indazol-7-yl]oxy-1- |
| | piperidyl]butyl]cyclohexoxy]-2-methyl-phenyl]pyridine-2-carboxylic acid |
| 327a | 6-(8-(benzo[d]thiazol-2-ylcarbamoyl)-3,4-dihydroisoquinolin-2(1H)-yl)-3-(3-(((1s,4r)- |
| | 4-(4-(4-((3-(2,6-dioxopiperidin-3-yl)-1-methyl-1H-indazol-7-yl)oxy)piperidin-1- |
| | yl)butyl)cyclohexyl)oxy)-2-methylphenyl)picolinic acid |
| 328 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-6-yl]piperazin-1- |
| | yl]propyl]cyclohexoxy]-2-(trifluoromethyl)phenyl]pyridine-2-carboxylic acid |
| 328a | 6-(8-(benzo[d]thiazol-2-ylcarbamoyl)-3,4-dihydroisoquinolin-2(1H)-yl)-3-(3-(((1s,4r)- |
| | 4-(3-(4-(3-(2,6-dioxopiperidin-3-yl)-1-methyl-1H-indazol-6-yl)piperazin-1- |
| | yl)propyl)cyclohexyl)oxy)-2-(trifluoromethyl)phenyl)picolinic acid |
| 329 | 6-[8-(1,3-benzothiazol-2-ylcarbamoyl)-3,4-dihydro-1H-isoquinolin-2-yl]-3-[3-[4-[4- |
| | [4-[3-(2,6-dioxo-3-piperidyl)-1-methyl-indazol-6-yl]piperazin-1- |
| | yl]butyl]cyclohexoxy]phenyl]pyridine-2-carboxylic acid |
| 329a | 6-(8-(benzo[d]thiazol-2-ylcarbamoyl)-3,4-dihydroisoquinolin-2(1H)-yl)-3-(3-(((1s,4r)- |
| | 4-(4-(4-(3-(2,6-dioxopiperidin-3-yl)-1-methyl-1H-indazol-6-yl)piperazin-1- |
| | yl)butyl)cyclohexyl)oxy)phenyl)picolinic acid |
| 330 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-6-yl]piperazin-1- |
| | yl]propyl]cyclohexoxy]phenyl]pyridine-2-carboxylic acid |
| 330a | 6-(8-(benzo[d]thiazol-2-ylcarbamoyl)-3,4-dihydroisoquinolin-2(1H)-yl)-3-(3-(((1s,4r)- |
| | 4-(3-(4-(3-(2,6-dioxopiperidin-3-yl)-1-methyl-1H-indazol-6-yl)piperazin-1- |
| | yl)propyl)cyclohexyl)oxy)phenyl)picolinic acid |
| 331 | 6-[8-(1,3-benzothiazol-2-ylcarbamoyl)-3,4-dihydro-1H-isoquinolin-2-yl]-3-[3-[4-[3- |
| | [3-[3-(2,6-dioxo-3-piperidyl)-1-methyl-indazol-6-yl]pyrrolidin-1- |
| | yl]propyl]cyclohexoxy]-2-methyl-phenyl]pyridine-2-carboxylic acid |
| 331a | 6-(8-(benzo[d]thiazol-2-ylcarbamoyl)-3,4-dihydroisoquinolin-2(1H)-yl)-3-(3- |
| | (((1S,4r)-4-(3-((3R*)-3-(3-(2,6-dioxopiperidin-3-yl)-1-methyl-1H-indazol-6- |
| | yl)pyrrolidin-1-yl)propyl)cyclohexyl)oxy)-2-methylphenyl)picolinic acid |
| 331b | 6-(8-(benzo[d]thiazol-2-ylcarbamoyl)-3,4-dihydroisoquinolin-2(1H)-yl)-3-(3- |
| | (((1R,4r)-4-(3-((3S*)-3-(3-(2,6-dioxopiperidin-3-yl)-1-methyl-1H-indazol-6- |
| | yl)pyrrolidin-1-yl)propyl)cyclohexyl)oxy)-2-methylphenyl)picolinic acid |
| 332 | 6-[8-(1,3-benzothiazol-2-ylcarbamoyl)-3,4-dihydro-1H-isoquinolin-2-yl]-3-[3-[4-[4- |
| | [3-[3-(2,6-dioxo-3-piperidyl)-1-methyl-indazol-6-yl]pyrrolidin-1- |
| | yl]butyl]cyclohexoxy]-2-methyl-phenyl]pyridine-2-carboxylic acid |
| 332a | 6-(8-(benzo[d]thiazol-2-ylcarbamoyl)-3,4-dihydroisoquinolin-2(1H)-yl)-3-(3- |
| | (((1S,4r)-4-(4-((3R*)-3-(3-(2,6-dioxopiperidin-3-yl)-1-methyl-1H-indazol-6- |
| | yl)pyrrolidin-1-yl)butyl)cyclohexyl)oxy)-2-methylphenyl)picolinic acid |
| 332b | 6-(8-(benzo[d]thiazol-2-ylcarbamoyl)-3,4-dihydroisoquinolin-2(1H)-yl)-3-(3- |
| | (((1R,4r)-4-(4-((3S*)-3-(3-(2,6-dioxopiperidin-3-yl)-1-methyl-1H-indazol-6- |
| | yl)pyrrolidin-1-yl)butyl)cyclohexyl)oxy)-2-methylphenyl)picolinic acid |
| 333 | 6-[8-(1,3-benzothiazol-2-ylcarbamoyl)-3,4-dihydro-1H-isoquinolin-2-yl]-3-[3-[4-[4- |
| | [4-[3-(2,6-dioxo-3-piperidyl)-1-methyl-indazol-6-yl]piperazin-1- |
| | yl]pentyl]cyclohexoxy]-2-methyl-phenyl]pyridine-2-carboxylic acid |
| 333a | 6-(8-(benzo[d]thiazol-2-ylcarbamoyl)-3,4-dihydroisoquinolin-2(1H)-yl)-3-(3- |
| | (((1R,4r)-4-((4S*)-4-(4-(3-(2,6-dioxopiperidin-3-yl)-1-methyl-1H-indazol-6- |
| | yl)piperazin-1-yl)pentyl)cyclohexyl)oxy)-2-methylphenyl)picolinic acid |
| 333b | 6-(8-(benzo[d]thiazol-2-ylcarbamoyl)-3,4-dihydroisoquinolin-2(1H)-yl)-3-(3- |
| | (((1S,4r)-4-((4R*)-4-(4-(3-(2,6-dioxopiperidin-3-yl)-1-methyl-1H-indazol-6- |
| | yl)piperazin-1-yl)pentyl)cyclohexyl)oxy)-2-methylphenyl)picolinic acid |
| 334 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1,7-dimethyl-indazol-6-yl]piperazin-1- |
| | yl]propyl]cyclohexoxy]-2-methyl-phenyl]pyridine-2-carboxylic acid |
| 334a | 6-(8-(benzo[d]thiazol-2-ylcarbamoyl)-3,4-dihydroisoquinolin-2(1H)-yl)-3-(3-(((1s,4r)- |
| | 4-(3-(4-(3-(2,6-dioxopiperidin-3-yl)-1,7-dimethyl-1H-indazol-6-yl)piperazin-1- |
| | yl)propyl)cyclohexyl)oxy)-2-methylphenyl)picolinic acid |
| 335 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1,5-dimethyl-indazol-6-yl]piperazin-1- |
| | yl]propyl]cyclohexoxy]-2-methyl-phenyl]pyridine-2-carboxylic acid |
| 335a | 6-(8-(benzo[d]thiazol-2-ylcarbamoyl)-3,4-dihydroisoquinolin-2(1H)-yl)-3-(3-(((1s,4r)- |
| | 4-(3-(4-(3-(2,6-dioxopiperidin-3-yl)-1,5-dimethyl-1H-indazol-6-yl)piperazin-1- |
| | yl)propyl)cyclohexyl)oxy)-2-methylphenyl)picolinic acid |
| 336 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-6-yl]-2,2-dimethyl-piperazin-1- |
| | yl]propyl]cyclohexoxy]-2-methyl-phenyl]pyridine-2-carboxylic acid |
| 336a | 6-(8-(benzo[d]thiazol-2-ylcarbamoyl)-3,4-dihydroisoquinolin-2(1H)-yl)-3-(3-(((1s,4r)- |
| | 4-(3-(4-(3-(2,6-dioxopiperidin-3-yl)-1-methyl-1H-indazol-6-yl)-2,2- |
| | dimethylpiperazin-1-yl)propyl)cyclohexyl)oxy)-2-methylphenyl)picolinic acid |
| 337 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-6-yl]oxy-1- |
| | piperidyl]propyl]cyclohexoxy]-2-methyl-phenyl]pyridine-2-carboxylic acid |
| 337a | 6-(8-(benzo[d]thiazol-2-ylcarbamoyl)-3,4-dihydroisoquinolin-2(1H)-yl)-3-(3-(((1s,4r)- |
| | 4-(3-(4-((3-(2,6-dioxopiperidin-3-yl)-1-methyl-1H-indazol-6-yl)oxy)piperidin-1- |
| | yl)propyl)cyclohexyl)oxy)-2-methylphenyl)picolinic acid |
| 338 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-7-yl]oxy-1- |
| | piperidyl]propyl]cyclohexoxy]-2-methyl-phenyl]pyridine-2-carboxylic acid |
| 338a | 6-(8-(benzo[d]thiazol-2-ylcarbamoyl)-3,4-dihydroisoquinolin-2(1H)-yl)-3-(3-(((1s,4r)- |
| | 4-(3-(4-((3-(2,6-dioxopiperidin-3-yl)-1-methyl-1H-indazol-7-yl)oxy)piperidin-1- |
| | yl)propyl)cyclohexyl)oxy)-2-methylphenyl)picolinic acid |
| 339 | 6-[8-(1,3-benzothiazol-2-ylcarbamoyl)-3,4-dihydro-1H-isoquinolin-2-yl]-3-[3-[4-[4- |
| | [4-[3-(2,6-dioxo-3-piperidyl)-1-methyl-indazol-6-yl]-3-(trifluoromethyl)piperazin-1- |
| | yl]butyl]cyclohexoxy]-2-methyl-phenyl]pyridine-2-carboxylic acid |
| 339a | 6-(8-(benzo[d]thiazol-2-ylcarbamoyl)-3,4-dihydroisoquinolin-2(1H)-yl)-3-(3-(((1s,4r)- |
| | 4-(4-((3R)-4-(3-(2,6-dioxopiperidin-3-yl)-1-methyl-1H-indazol-6-yl)-3- |
| | (trifluoromethyl)piperazin-1-yl)butyl)cyclohexyl)oxy)-2-methylphenyl)picolinic acid |
| 339b | 6-(8-(benzo[d]thiazol-2-ylcarbamoyl)-3,4-dihydroisoquinolin-2(1H)-yl)-3-(3- |
| | (((1R,4r)-4-(4-((3S)-4-(3-(2,6-dioxopiperidin-3-yl)-1-methyl-1H-indazol-6-yl)-3- |
| | (trifluoromethyl)piperazin-1-yl)butyl)cyclohexyl)oxy)-2-methylphenyl)picolinic acid |
| 340 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-6-yl]piperazin-1-yl]-3-methyl- |
| | butyl]cyclohexoxy]-2-methyl-phenyl]pyridine-2-carboxylic acid |
| 340a | 6-(8-(benzo[d]thiazol-2-ylcarbamoyl)-3,4-dihydroisoquinolin-2(1H)-yl)-3-(3-(((1s,4r)- |
| | 4-(3-(4-(3-(2,6-dioxopiperidin-3-yl)-1-methyl-1H-indazol-6-yl)piperazin-1-yl)-3- |
| | methylbutyl)cyclohexyl)oxy)-2-methylphenyl)picolinic acid |
| 341 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-7-yl]piperazin-1-yl]-3-methyl- |
| | butyl]cyclohexoxy]-2-methyl-phenyl]pyridine-2-carboxylic acid |
| 341a | 6-(8-(benzo[d]thiazol-2-ylcarbamoyl)-3,4-dihydroisoquinolin-2(1H)-yl)-3-(3-(((1s, |
| | 4r)-4-(3-(4-(3-(2,6-dioxopiperidin-3-yl)-1-methyl-1H-indazol-7-yl)piperazin-1-yl)-3- |
| | methylbutyl)cyclohexyl)oxy)-2-methylphenyl)picolinic acid |
| 342 | 6-[8-(1,3-benzothiazol-2-ylcarbamoyl)-3,4-dihydro-1H-isoquinolin-2-yl]-3-[3-[4-[4- |
| | [4-[3-(2,6-dioxo-3-piperidyl)-1-methyl-indazol-7-yl]piperazin-1-yl]-4-methyl- |
| | pentyl]cyclohexoxy]-2-methyl-phenyl]pyridine-2-carboxylic acid |
| 342a | 6-(8-(benzo[d]thiazol-2-ylcarbamoyl)-3,4-dihydroisoquinolin-2(1H)-yl)-3-(3-(((1s,4r)- |
| | 4-(4-(4-(3-(2,6-dioxopiperidin-3-yl)-1-methyl-1H-indazol-7-yl)piperazin-1-yl)-4- |
| | methylpentyl)cyclohexyl)oxy)-2-methylphenyl)picolinic acid |
| 343 | 6-[8-(1,3-benzothiazol-2-ylcarbamoyl)-3,4-dihydro-1H-isoquinolin-2-yl]-3-[3-[3-[1- |
| | [2-[[3-(2,6-dioxo-3-piperidyl)-1-methyl-indazol-6-yl]amino]-2-oxo-ethyl]-2-methyl-4- |
| | piperidyl]propoxy]-2-methyl-phenyl]pyridine-2-carboxylic acid |
| 343a | 6-(8-(benzo[d]thiazol-2-ylcarbamoyl)-3,4-dihydroisoquinolin-2(1H)-yl)-3-(3-(3- |
| | ((2R,4R)-1-(2-((3-(2,6-dioxopiperidin-3-yl)-1-methyl-1H-indazol-6-yl)amino)-2- |
| | oxoethyl)-2-methylpiperidin-4-yl)propoxy)-2-methylphenyl)picolinic acid |
| 343b | 6-[8-(1,3-benzothiazol-2-ylcarbamoyl)-3,4-dihydro-1H-isoquinolin-2-yl]-3-[3-[3- |
| | [(2R,4S)-1-[2-[[3-(2,6-dioxo-3-piperidyl)-1-methyl-indazol-6-yl]amino]-2-oxo-ethyl]- |
| | 2-methyl-4-piperidyl]propoxy]-2-methyl-phenyl]pyridine-2-carboxylic acid |
| 343c | 6-(8-(benzo[d]thiazol-2-ylcarbamoyl)-3,4-dihydroisoquinolin-2(1H)-yl)-3-(3-(3- |
| | ((2S,4R)-1-(2-((3-(2,6-dioxopiperidin-3-yl)-1-methyl-1H-indazol-6-yl)amino)-2- |
| | oxoethyl)-2-methylpiperidin-4-yl)propoxy)-2-methylphenyl)picolinic acid |
| 343d | 6-[8-(1,3-benzothiazol-2-ylcarbamoyl)-3,4-dihydro-1H-isoquinolin-2-yl]-3-[3-[3- |
| | [(2S,4S)-1-[2-[[3-(2,6-dioxo-3-piperidyl)-1-methyl-indazol-6-yl]amino]-2-oxo-ethyl]- |
| | 2-methyl-4-piperidyl]propoxy]-2-methyl-phenyl]pyridine-2-carboxylic acid |
| 344 | 6-[8-(1,3-benzothiazol-2-ylcarbamoyl)-3,4-dihydro-1H-isoquinolin-2-yl]-3-[4-[3-[1- |
| | [2-[[3-(2,6-dioxo-3-piperidyl)-1-methyl-indazol-6-yl]amino]-2-oxo-ethyl]-2-methyl-4- |
| | piperidyl]propoxy]-2-methyl-phenyl]pyridine-2-carboxylic acid |
| 344a | 6-[8-(1,3-benzothiazol-2-ylcarbamoyl)-3,4-dihydro-1H-isoquinolin-2-yl]-3-[4-[3- |
| | [(2S,4S)-1-[2-[[3-(2,6-dioxo-3-piperidyl)-1-methyl-indazol-6-yl]amino]-2-oxo-ethyl]- |
| | 2-methyl-4-piperidyl]propoxy]-2-methyl-phenyl]pyridine-2-carboxylic acid |
| 344b | 6-[8-(1,3-benzothiazol-2-ylcarbamoyl)-3,4-dihydro-1H-isoquinolin-2-yl]-3-[4-[3- |
| | [(2R,4R)-1-[2-[[3-(2,6-dioxo-3-piperidyl)-1-methyl-indazol-6-yl]amino]-2-oxo-ethyl]- |
| | 2-methyl-4-piperidyl]propoxy]-2-methyl-phenyl]pyridine-2-carboxylic acid |
| 344c | 6-[8-(1,3-benzothiazol-2-ylcarbamoyl)-3,4-dihydro-1H-isoquinolin-2-yl]-3-[4-[3- |
| | [(2R,4S)-1-[2-[[3-(2,6-dioxo-3-piperidyl)-1-methyl-indazol-6-yl]amino]-2-oxo-ethyl]- |
| | 2-methyl-4-piperidyl]propoxy]-2-methyl-phenyl]pyridine-2-carboxylic acid |
| 344d | 6-(8-(benzo[d]thiazol-2-ylcarbamoyl)-3,4-dihydroisoquinolin-2(1H)-yl)-3-(4-(3- |
| | ((2S,4R)-1-(2-((3-(2,6-dioxopiperidin-3-yl)-1-methyl-1H-indazol-6-yl)amino)-2- |
| | oxoethyl)-2-methylpiperidin-4-yl)propoxy)-2-methylphenyl)picolinic acid |
| 345 | 6-[8-(1,3-benzothiazol-2-ylcarbamoyl)-3,4-dihydro-1H-isoquinolin-2-yl]-3-[3-[4-[4- |
| | [4-[3-(2,6-dioxo-3-piperidyl)-1-methyl-indazol-7-yl]-2-methyl-piperazin-1- |
| | yl]butyl]cyclohexoxy]-2-methyl-phenyl]pyridine-2-carboxylic acid |
| 345a | 6-(8-(benzo[d]thiazol-2-ylcarbamoyl)-3,4-dihydroisoquinolin-2(1H)-yl)-3-(3-(((1s,4r)- |
| | 4-(4-((2R)-4-(3-(2,6-dioxopiperidin-3-yl)-1-methyl-1H-indazol-7-yl)-2- |
| | methylpiperazin-1-yl)butyl)cyclohexyl)oxy)-2-methylphenyl)picolinic acid |
| 346 | 6-[8-(1,3-benzothiazol-2-ylcarbamoyl)-3,4-dihydro-1H-isoquinolin-2-yl]-3-[3-[4-[4- |
| | [4-[3-(2,6-dioxo-3-piperidyl)-1-methyl-indazol-6-yl]-2-(trifluoromethyl)piperazin-1- |
| | yl]butyl]cyclohexoxy]-2-methyl-phenyl]pyridine-2-carboxylic acid |
| 346a | 6-(8-(benzo[d]thiazol-2-ylcarbamoyl)-3,4-dihydroisoquinolin-2(1H)-yl)-3-(3-(((1s,4r)- |
| | 4-(4-((2R)-4-(3-(2,6-dioxopiperidin-3-yl)-1-methyl-1H-indazol-6-yl)-2- |
| | (trifluoromethyl)piperazin-1-yl)butyl)cyclohexyl)oxy)-2-methylphenyl)picolinic acid |
| 346b | 6-(8-(benzo[d]thiazol-2-ylcarbamoyl)-3,4-dihydroisoquinolin-2(1H)-yl)-3-(3-(((1r,4r)- |
| | 4-(4-((2S)-4-(3-(2,6-dioxopiperidin-3-yl)-1-methyl-1H-indazol-6-yl)-2- |
| | (trifluoromethyl)piperazin-1-yl)butyl)cyclohexyl)oxy)-2-methylphenyl)picolinic acid |
| 347 | 6-[8-(1,3-benzothiazol-2-ylcarbamoyl)-3,4-dihydro-1H-isoquinolin-2-yl]-3-[3-[4-[3- |
| | [4-[3-(2,6-dioxo-3-piperidyl)-1-methyl-indazol-6-yl]-2-(trifluoromethyl)piperazin-1- |
| | yl]propyl]cyclohexoxy]-2-methyl-phenyl]pyridine-2-carboxylic acid |
| 347a | 6-(8-(benzo[d]thiazol-2-ylcarbamoyl)-3,4-dihydroisoquinolin-2(1H)-yl)-3-(3-(((1s,4r)- |
| | 4-(3-((2R)-4-(3-(2,6-dioxopiperidin-3-yl)-1-methyl-1H-indazol-6-yl)-2- |
| | (trifluoromethyl)piperazin-1-yl)propyl)cyclohexyl)oxy)-2-methylphenyl)picolinic acid |
| 347b | 6-(8-(benzo[d]thiazol-2-ylcarbamoyl)-3,4-dihydroisoquinolin-2(1H)-yl)-3-(3-(((1r,4r)- |
| | 4-(3-((2S)-4-(3-(2,6-dioxopiperidin-3-yl)-1-methyl-1H-indazol-6-yl)-2- |
| | (trifluoromethyl)piperazin-1-yl)propyl)cyclohexyl)oxy)-2-methylphenyl)picolinic acid |
| 349 | 6-[8-(1,3-benzothiazol-2-ylcarbamoyl)-3,4-dihydro-1H-isoquinolin-2-yl]-3-[3-[4-[4- |
| | [4-[3-(2,6-dioxo-3-piperidyl)-1-methyl-indazol-7-yl]piperazin-1- |
| | yl]pentyl]cyclohexoxy]-2-methyl-phenyl]pyridine-2-carboxylic acid |
| 349a | 6-(8-(benzo[d]thiazol-2-ylcarbamoyl)-3,4-dihydroisoquinolin-2(1H)-yl)-3-(3- |
| | (((1R,4r)-4-((4S*)-4-(4-(3-(2,6-dioxopiperidin-3-yl)-1-methyl-1H-indazol-7- |
| | yl)piperazin-1-yl)pentyl)cyclohexyl)oxy)-2-methylphenyl)picolinic acid |
| 349b | 6-(8-(benzo[d]thiazol-2-ylcarbamoyl)-3,4-dihydroisoquinolin-2(1H)-yl)-3-(3- |
| | (((1S,4r)-4-((4R*)-4-(4-(3-(2,6-dioxopiperidin-3-yl)-1-methyl-1H-indazol-7- |
| | yl)piperazin-1-yl)pentyl)cyclohexyl)oxy)-2-methylphenyl)picolinic acid |
| 350 | 6-[8-(1,3-benzothiazol-2-ylcarbamoyl)-3,4-dihydro-1H-isoquinolin-2-yl]-3-[3-[4-[4- |
| | [4-[3-(2,6-dioxo-3-piperidyl)-1-methyl-indazol-6-yl]piperazin-1-yl]-4-methyl- |
| | pentyl]cyclohexoxy]-2-methyl-phenyl]pyridine-2-carboxylic acid |
| 350a | 6-(8-(benzo[d]thiazol-2-ylcarbamoyl)-3,4-dihydroisoquinolin-2(1H)-yl)-3-(3-(((1s,4r)- |
| | 4-(4-(4-(3-(2,6-dioxopiperidin-3-yl)-1-methyl-1H-indazol-6-yl)piperazin-1-yl)-4- |
| | methylpentyl)cyclohexyl)oxy)-2-methylphenyl)picolinic acid |
| 351 | 6-[8-(1,3-benzothiazol-2-ylcarbamoyl)-3,4-dihydro-1H-isoquinolin-2-yl]-3-[3-[4-[4- |
| | [3-[3-(2,6-dioxo-3-piperidyl)-1-methyl-indazol-7-yl]-1-piperidyl]butyl]cyclohexoxy]- |
| | 2-methyl-phenyl]pyridine-2-carboxylic acid |
| 351a | 6-(8-(benzo[d]thiazol-2-ylcarbamoyl)-3,4-dihydroisoquinolin-2(1H)-yl)-3-(3- |
| | (((1S,4r)-4-(4-((3R*)-3-(3-(2,6-dioxopiperidin-3-yl)-1-methyl-1H-indazol-7- |
| | yl)piperidin-1-yl)butyl)cyclohexyl)oxy)-2-methylphenyl)picolinic acid |
| 351b | 6-(8-(benzo[d]thiazol-2-ylcarbamoyl)-3,4-dihydroisoquinolin-2(1H)-yl)-3-(3- |
| | (((1R,4r)-4-(4-((3S*)-3-(3-(2,6-dioxopiperidin-3-yl)-1-methyl-1H-indazol-7- |
| | yl)piperidin-1-yl)butyl)cyclohexyl)oxy)-2-methylphenyl)picolinic acid |
| 352 | 6-[8-(1,3-benzothiazol-2-ylcarbamoyl)-3,4-dihydro-1H-isoquinolin-2-yl]-3-[3-[4-[4- |
| | [3-[3-(2,6-dioxo-3-piperidyl)-1-methyl-indazol-6-yl]-1-piperidyl]butyl]cyclohexoxy]- |
| | 2-methyl-phenyl]pyridine-2-carboxylic acid |
| 352a | 6-(8-(benzo[d]thiazol-2-ylcarbamoyl)-3,4-dihydroisoquinolin-2(1H)-yl)-3-(3- |
| | (((1S,4r)-4-(4-((3R*)-3-(3-(2,6-dioxopiperidin-3-yl)-1-methyl-1H-indazol-6- |
| | yl)piperidin-1-yl)butyl)cyclohexyl)oxy)-2-methylphenyl)picolinic acid |
| 352b | 6-(8-(benzo[d]thiazol-2-ylcarbamoyl)-3,4-dihydroisoquinolin-2(1H)-yl)-3-(3- |
| | (((1R,4r)-4-(4-((3S*)-3-(3-(2,6-dioxopiperidin-3-yl)-1-methyl-1H-indazol-6- |
| | yl)piperidin-1-yl)butyl)cyclohexyl)oxy)-2-methylphenyl)picolinic acid |
| 353 | 6-(8-(benzo[d]thiazol-2-ylcarbamoyl)-3,4-dihydroisoquinolin-2(1H)-yl)-3-(3-((4-(3- |
| | (1-(3-(2,6-dioxopiperidin-3-yl)-1-methyl-1H-indazol-7-yl)piperidin-4- |
| | yl)propyl)cyclohexyl)oxy)-2-methylphenyl)picolinic acid |
| 353a | 6-(8-(benzo[d]thiazol-2-ylcarbamoyl)-3,4-dihydroisoquinolin-2(1H)-yl)-3-(3-(((1r,4s)- |
| | 4-(3-(1-(3-(2,6-dioxopiperidin-3-yl)-1-methyl-1H-indazol-7-yl)piperidin-4- |
| | yl)propyl)cyclohexyl)oxy)-2-methylphenyl)picolinic acid |
| 353b | 6-(8-(benzo[d]thiazol-2-ylcarbamoyl)-3,4-dihydroisoquinolin-2(1H)-yl)-3-(3-(((1s,4r)- |
| | 4-(3-(1-(3-(2,6-dioxopiperidin-3-yl)-1-methyl-1H-indazol-7-yl)piperidin-4- |
| | yl)propyl)cyclohexyl)oxy)-2-methylphenyl)picolinic acid |
| 354 | 6-(8-(benzo[d]thiazol-2-ylcarbamoyl)-3,4-dihydroisoquinolin-2(1H)-yl)-3-(3-((4-(4- |
| | (4-(3-(2,6-dioxopiperidin-3-yl)-1-methyl-1H-indazol-6-yl)-2,6-dimethylpiperazin-1- |
| | yl)butyl)cyclohexyl)oxy)-2-methylphenyl)picolinic acid |
| 354a | 6-(8-(benzo[d]thiazol-2-ylcarbamoyl)-3,4-dihydroisoquinolin-2(1H)-yl)-3-(3-(((1s,4r)- |
| | 4-(4-((2S,6R)-4-(3-(2,6-dioxopiperidin-3-yl)-1-methyl-1H-indazol-6-yl)-2,6- |
| | dimethylpiperazin-1-yl)butyl)cyclohexyl)oxy)-2-methylphenyl)picolinic acid |
| 354b | 6-(8-(benzo[d]thiazol-2-ylcarbamoyl)-3,4-dihydroisoquinolin-2(1H)-yl)-3-(3-(((1r,4r)- |
| | 4-(4-((2S,6S)-4-(3-(2,6-dioxopiperidin-3-yl)-1-methyl-1H-indazol-6-yl)-2,6- |
| | dimethylpiperazin-1-yl)butyl)cyclohexyl)oxy)-2-methylphenyl)picolinic acid |
| 355 | 6-[8-(1,3-benzothiazol-2-ylcarbamoyl)-3,4-dihydro-1H-isoquinolin-2-yl]-3-[4-[3-[1- |
| | [2-[[3-(2,6-dioxo-3-piperidyl)-1-methyl-indazol-6-yl]amino]-2-oxo-ethyl]-4-fluoro-4- |
| | piperidyl]propoxy]-2-methyl-phenyl]pyridine-2-carboxylic acid |
| 356 | 6-(8-(benzo[d]thiazol-2-ylcarbamoyl)-3,4-dihydroisoquinolin-2(1H)-yl)-3-(4-(3-(1-(2- |
| | ((3-(2,6-dioxopiperidin-3-yl)-7-fluoro-1-methyl-1H-indazol-6-yl)amino)-2- |
| | oxoethyl)piperidin-4-yl)propoxy)-2-methylphenyl)picolinic acid |
| 357 | 6-[8-(1,3-benzothiazol-2-ylcarbamoyl)-3,4-dihydro-1H-isoquinolin-2-yl]-3-[3-[3-[1- |
| | [2-[[3-(2,6-dioxo-3-piperidyl)-1-methyl-indazol-6-yl]amino]-2-oxo-ethyl]-4-fluoro-4- |
| | piperidyl]propoxy]-2-methyl-phenyl]pyridine-2-carboxylic acid |

Certain examples of Formula (I) or (II) compounds were synthesized using methods involving resolution of stereoisomeric mixture(s) (e.g., SFC separation of stereoisomers). In Table C1, the resolved stereogenic centers in these compounds are labelled with the “or1” or “or2” enhanced stereochemical notations. In some instances, the stereoisomeric resolutions were performed during the last step of the synthesis, thereby providing the individual stereoisomers of the Formula (I) or (II) compounds. Alternatively, in some other instances, the resolutions were performed on an intermediate or starting material, wherein each of the constituent stereoisomers of the intermediate or starting material could be separately subjected to the subsequent steps of the synthesis to provide the respective Formula (I) or (II) compounds as separate stereoisomers. Methods of resolution and correlation between resolved intermediates and Formula (I) or (II) compounds are disclosed in the examples herein and in Table P1. A person of ordinary skill in the art would understand that, under either approach for stereoisomeric resolution, stereoisomers having both (R)- and (S)-configurations at a resolved stereogenic center are provided. See Table C₃, wherein Table C1 compounds whose stereoisomers contain the or1 notations are provided in non-stereogenic form, followed by the respective stereoisomers having the (R)- and (S)-configurations.

TABLE C3

| No. | Compound Structure |
|---|---|
| 262 | 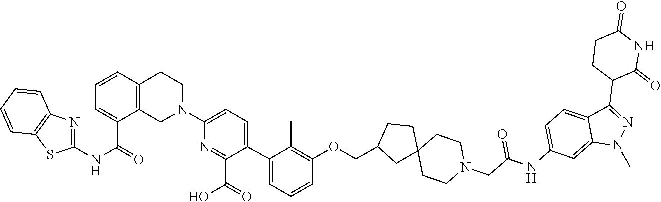 [SMILES: Cc1c(OCC2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| | 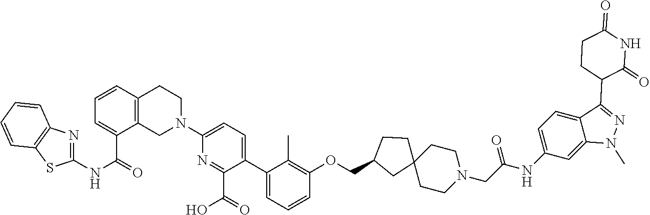 [SMILES: Cc1c(OC[C@H]2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| | 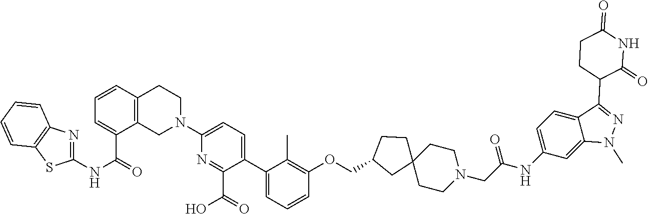 [SMILES: Cc1c(OC[C@@H]2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| 263 | 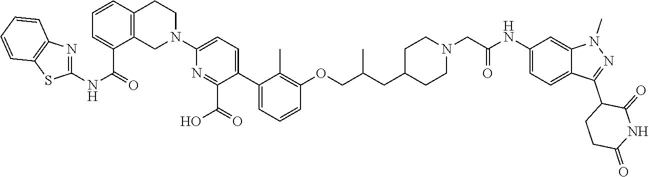 [SMILES: Cc1c(OCC(C)CC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| | 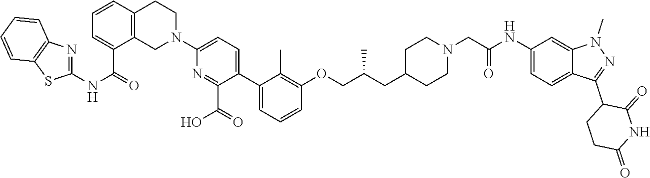 [SMILES: Cc1c(OC[C@H](C)CC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| | 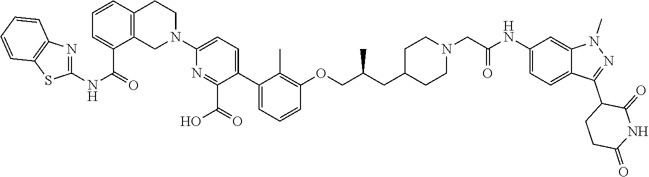 [SMILES: Cc1c(OC[C@@H](C)CC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] |
| 266 | 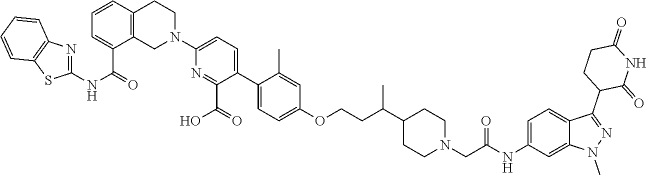 [SMILES: Cc1cc(OCCC(C)C2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| | 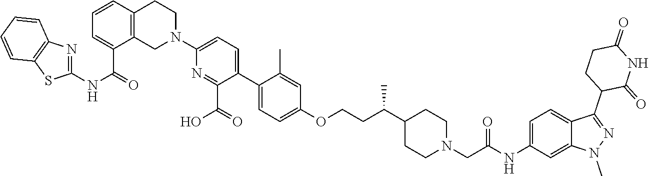 [SMILES: Cc1cc(OCC[C@H](C)C2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| | 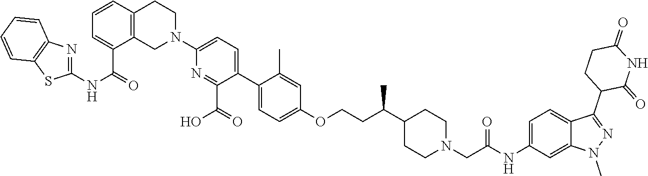 [SMILES: Cc1cc(OCC[C@@H](C)C2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 268 | 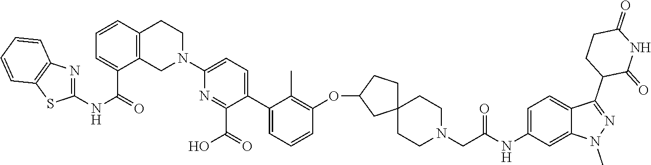 [SMILES: Cc1c(OC2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| | 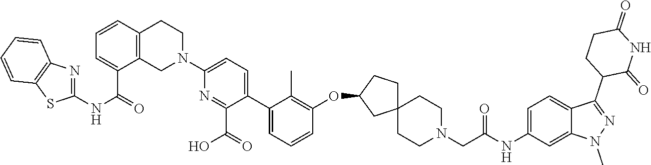 [SMILES: Cc1c(O[C@H]2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| | 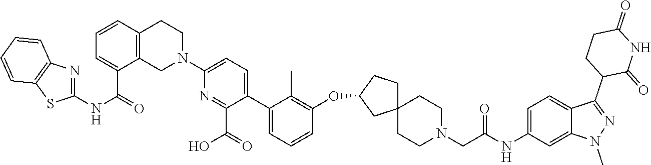 [SMILES: Cc1c(O[C@@H]2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| 271 | 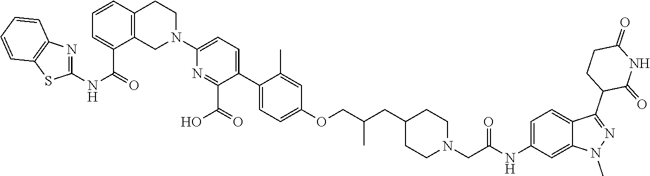 [SMILES: Cc1cc(OCC(C)CC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| | 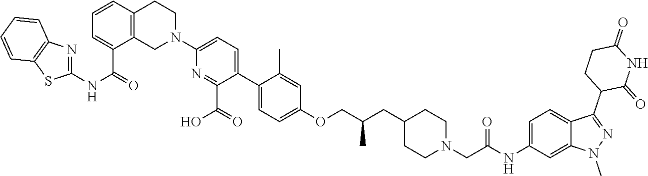 [SMILES: Cc1cc(OC[C@H](C)CC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| | 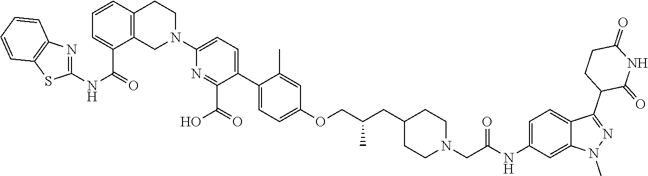 [SMILES: Cc1cc(OC[C@@H](C)CC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 272 | 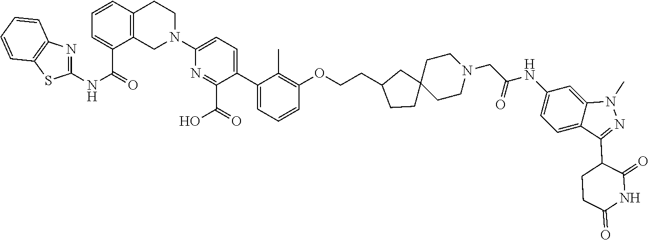 [SMILES: Cc1c(OCCC2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| | 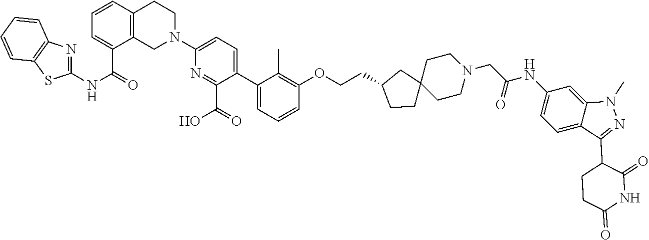 [SMILES: Cc1c(OCC[C@H]2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| | 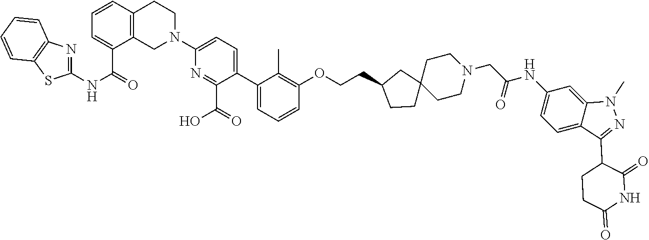 [SMILES: Cc1c(OCC[C@@H]2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O] |
| 276 | 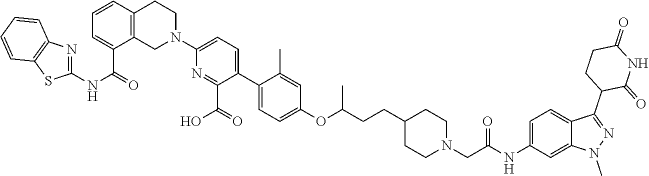 [SMILES: Cc1cc(OC(C)CCC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| | 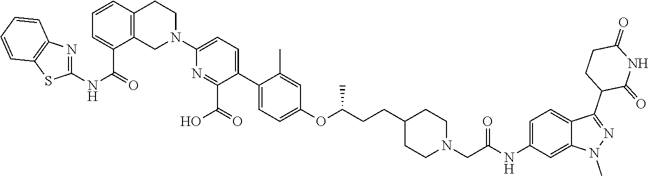 [SMILES: Cc1cc(O[C@H](C)CCC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| | 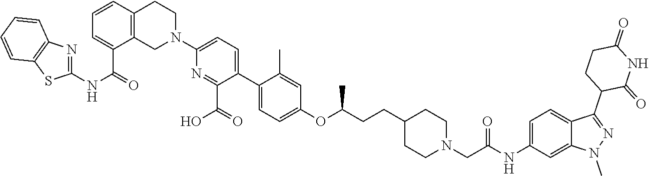 [SMILES: Cc1cc(O[C@@H](C)CCC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] |
| 281 | 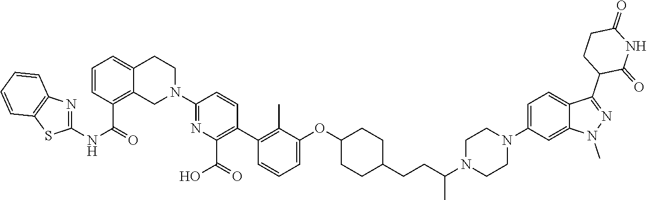 [SMILES: Cc1c(OC2CCC(CCC(C)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| | 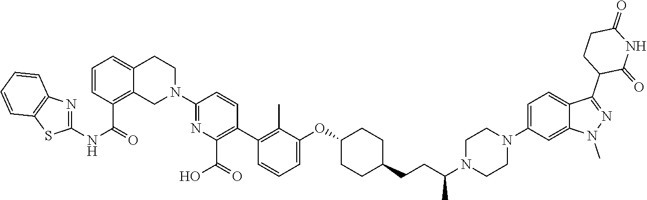 [SMILES: Cc1c(O[C@H]2CC[C@H](CC[C@H](C)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| | 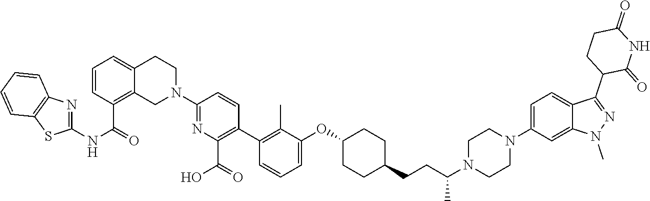 [SMILES: Cc1c(O[C@H]2CC[C@H](CC[C@@H](C)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 282 | 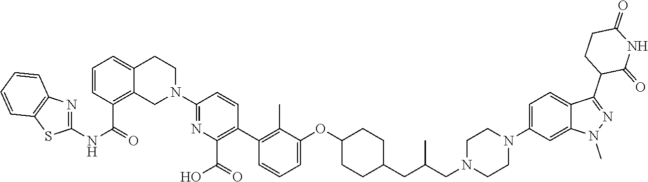 [SMILES: Cc1c(OC2CCC(CC(C)CN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| | 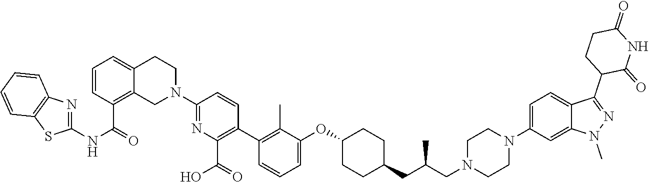 [SMILES: Cc1c(O[C@H]2CC[C@H](C[C@@H](C)CN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| | 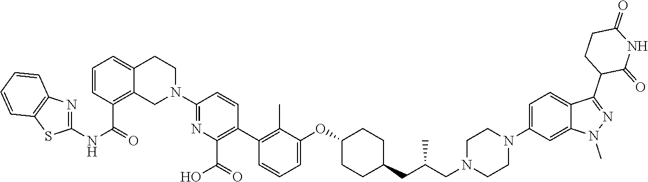 [SMILES: Cc1c(O[C@H]2CC[C@H](C[C@H](C)CN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 283 | 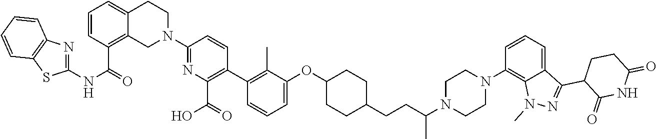 [SMILES: Cc1c(OC2CCC(CCC(C)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| | 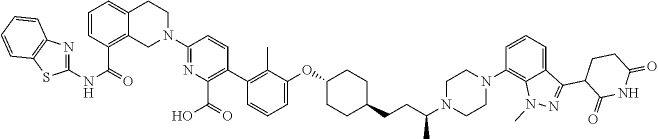 [SMILES: Cc1c(O[C@H]2CC[C@H](CC[C@H](C)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| | 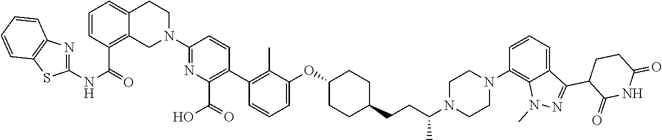 [SMILES: Cc1c(O[C@H]2CC[C@H](CC[C@@H](C)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 284 | 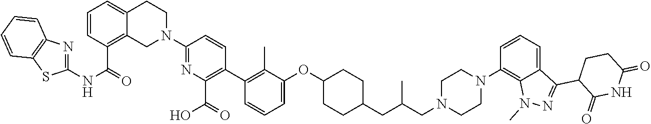 [SMILES: Cc1c(OC2CCC(CC(C)CN3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| | 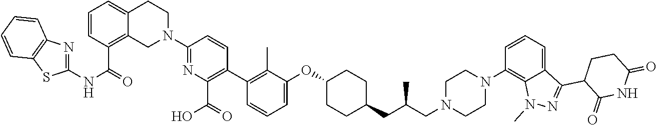 [SMILES: Cc1c(O[C@H]2CC[C@H](C[C@@H](C)CN3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| | 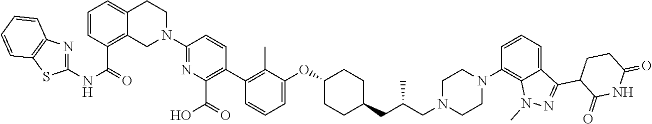 [SMILES: Cc1c(O[C@H]2CC[C@H](C[C@H](C)CN3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 289 | 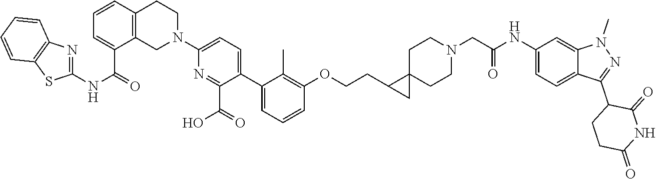 [SMILES: Cc1c(OCCC2CC23CCN(CC(=O)Nc2ccc4c(C5CCC(=O)NC5=O)nn(C)c4c2)CC3)cccc1-c1ccc(N2CCc3cccc(C(=O)Nc4nc5ccccc5s4)c3C2)nc1C(=O)O] |
| | 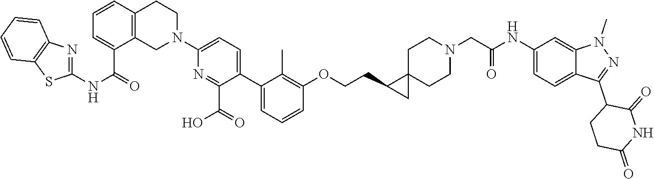 [SMILES: Cc1c(OCC[C@@H]2CC23CCN(CC(=O)Nc2ccc4c(C5CCC(=O)NC5=O)nn(C)c4c2)CC3)cccc1-c1ccc(N2CCc3cccc(C(=O)Nc4nc5ccccc5s4)c3C2)nc1C(=O)O] |
| | 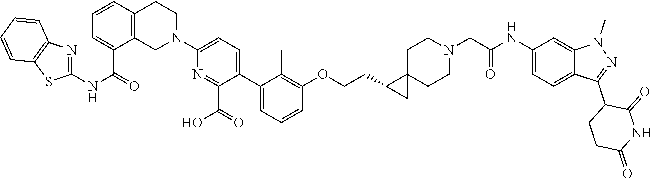 [SMILES: Cc1c(OCC[C@H]2CC23CCN(CC(=O)Nc2ccc4c(C5CCC(=O)NC5=O)nn(C)c4c2)CC3)cccc1-c1ccc(N2CCc3cccc(C(=O)Nc4nc5ccccc5s4)c3C2)nc1C(=O)O] |
| 307 | 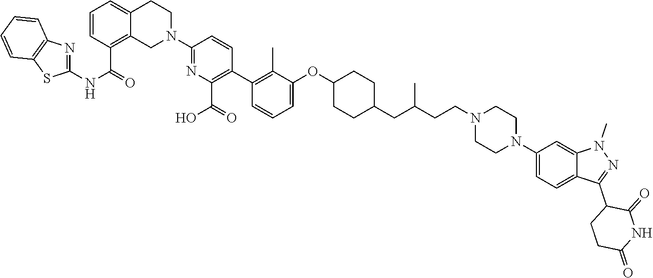 [SMILES: Cc1c(OC2CCC(CC(C)CCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| | 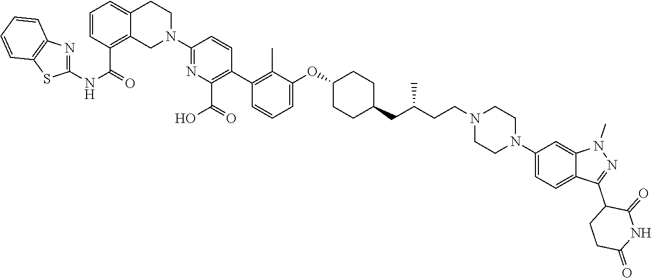 [SMILES: Cc1c(O[C@H]2CC[C@H](C[C@H](C)CCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| | 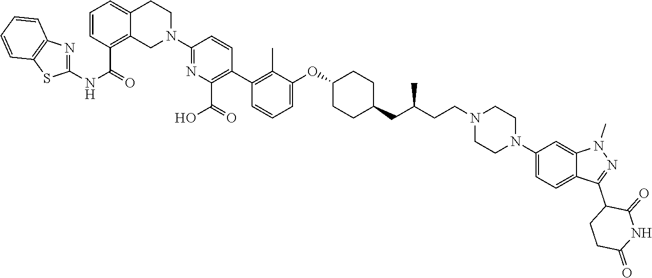 [SMILES: Cc1c(O[C@H]2CC[C@H](C[C@@H](C)CCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 308 | 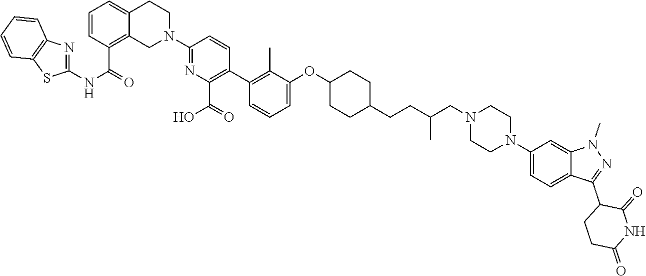 [SMILES: Cc1c(OC2CCC(CCC(C)CN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| | 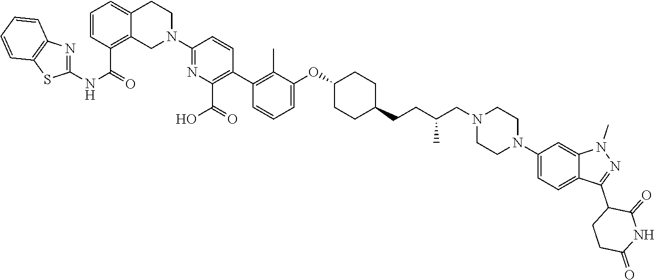 [SMILES: Cc1c(O[C@H]2CC[C@H](CC[C@@H](C)CN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| | 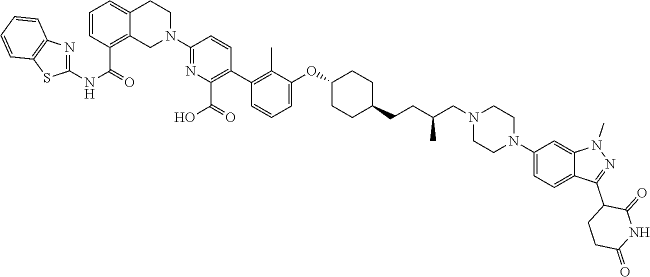 [SMILES: Cc1c(O[C@H]2CC[C@H](CC[C@H](C)CN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 310 | 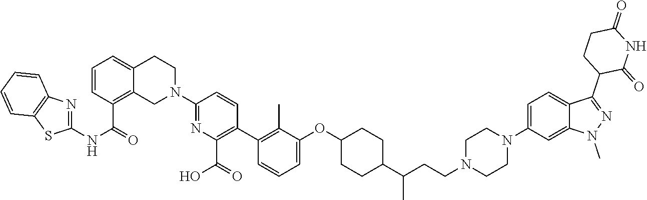 [SMILES: Cc1c(OC2CCC(C(C)CCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| | 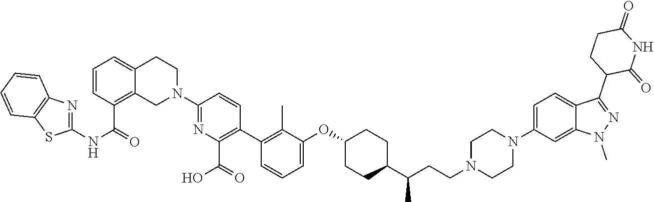 [SMILES: Cc1c(O[C@H]2CC[C@H]([C@H](C)CCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| | 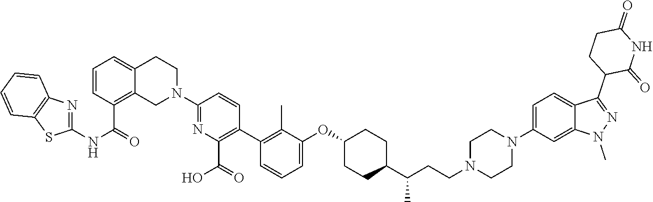 [SMILES: Cc1c(O[C@H]2CC[C@H]([C@@H](C)CCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 320 | 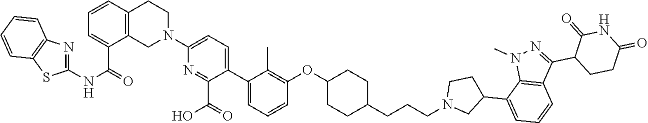 [SMILES: Cc1c(OC2CCC(CCCN3CCC(c4cccc5c(C6CCC(=O)NC6=O)nn(C)c45)C3)CC2)cccc1-c1ccc(N2CCc3cccc(C(=O)Nc4nc5ccccc5s4)c3C2)nc1C(=O)O] |
| | 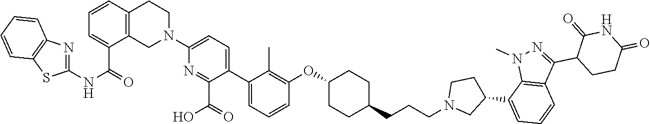 [SMILES: Cc1c(O[C@H]2CC[C@H](CCCN3CC[C@H](c4cccc5c(C6CCC(=O)NC6=O)nn(C)c45)C3)CC2)cccc1-c1ccc(N2CCc3cccc(C(=O)Nc4nc5ccccc5s4)c3C2)nc1C(=O)O] |
| | 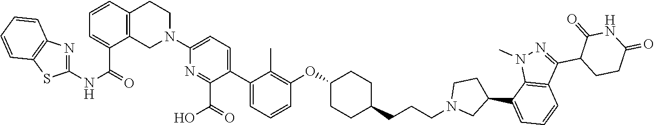 [SMILES: Cc1c(O[C@H]2CC[C@H](CCCN3CC[C@@H](c4cccc5c(C6CCC(=O)NC6=O)nn(C)c45)C3)CC2)cccc1-c1ccc(N2CCc3cccc(C(=O)Nc4nc5ccccc5s4)c3C2)nc1C(=O)O] |
| 321 | 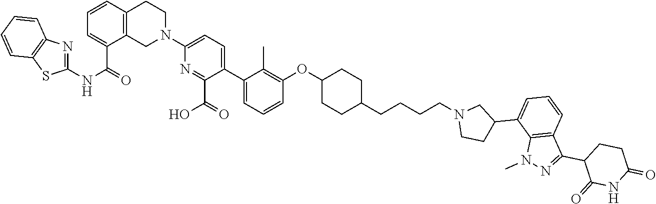 [SMILES: Cc1c(OC2CCC(CCCCN3CCC(c4cccc5c(C6CCC(=O)NC6=O)nn(C)c45)C3)CC2)cccc1-c1ccc(N2CCc3cccc(C(=O)Nc4nc5ccccc5s4)c3C2)nc1C(=O)O] |
| | 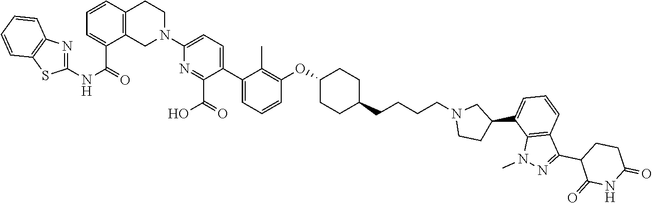 [SMILES: Cc1c(O[C@H]2CC[C@H](CCCCN3CC[C@H](c4cccc5c(C6CCC(=O)NC6=O)nn(C)c45)C3)CC2)cccc1-c1ccc(N2CCc3cccc(C(=O)Nc4nc5ccccc5s4)c3C2)nc1C(=O)O] |
| | 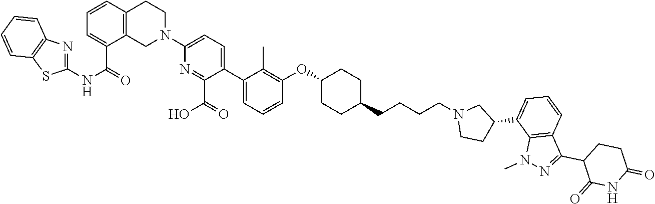 [SMILES: Cc1c(O[C@H]2CC[C@H](CCCCN3CC[C@@H](c4cccc5c(C6CCC(=O)NC6=O)nn(C)c45)C3)CC2)cccc1-c1ccc(N2CCc3cccc(C(=O)Nc4nc5ccccc5s4)c3C2)nc1C(=O)O] |
| 331 | 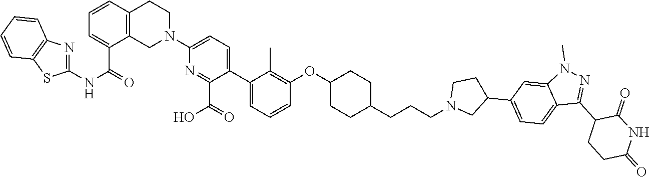 [SMILES: Cc1c(OC2CCC(CCCN3CCC(c4ccc5c(C6CCC(=O)NC6=O)nn(C)c5c4)C3)CC2)cccc1-c1ccc(N2CCc3cccc(C(=O)Nc4nc5ccccc5s4)c3C2)nc1C(=O)O] |
| | 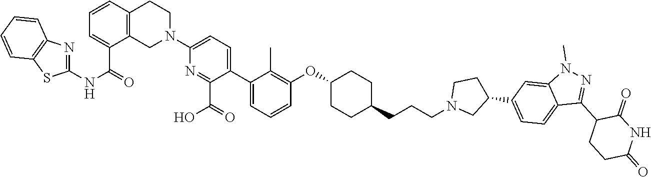 [SMILES: Cc1c(O[C@H]2CC[C@H](CCCN3CC[C@H](c4ccc5c(C6CCC(=O)NC6=O)nn(C)c5c4)C3)CC2)cccc1-c1ccc(N2CCc3cccc(C(=O)Nc4nc5ccccc5s4)c3C2)nc1C(=O)O] |
| | 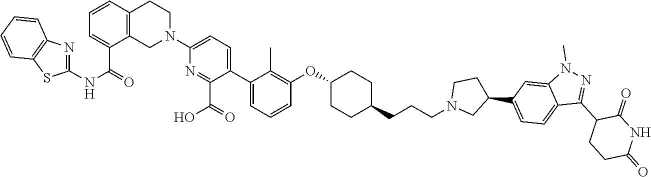 [SMILES: Cc1c(O[C@H]2CC[C@H](CCCN3CC[C@@H](c4ccc5c(C6CCC(=O)NC6=O)nn(C)c5c4)C3)CC2)cccc1-c1ccc(N2CCc3cccc(C(=O)Nc4nc5ccccc5s4)c3C2)nc1C(=O)O] |
| 332 | 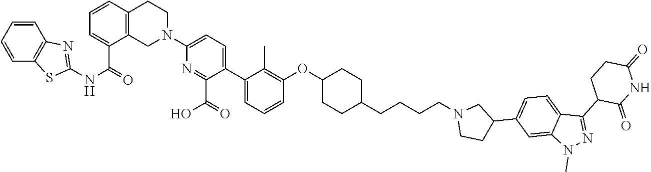 [SMILES: Cc1c(OC2CCC(CCCCN3CCC(c4ccc5c(C6CCC(=O)NC6=O)nn(C)c5c4)C3)CC2)cccc1-c1ccc(N2CCc3cccc(C(=O)Nc4nc5ccccc5s4)c3C2)nc1C(=O)O] |
| | 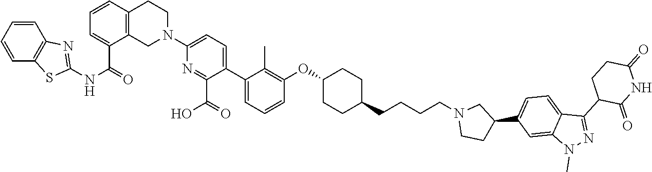 [SMILES: Cc1c(O[C@H]2CC[C@H](CCCCN3CC[C@H](c4ccc5c(C6CCC(=O)NC6=O)nn(C)c5c4)C3)CC2)cccc1-c1ccc(N2CCc3cccc(C(=O)Nc4nc5ccccc5s4)c3C2)nc1C(=O)O] |
| | 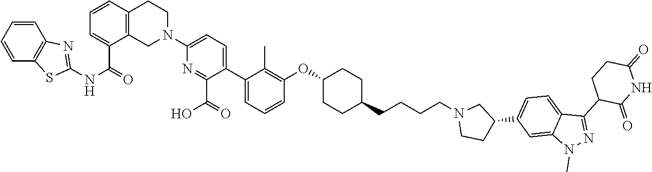 [SMILES: Cc1c(O[C@H]2CC[C@H](CCCCN3CC[C@@H](c4ccc5c(C6CCC(=O)NC6=O)nn(C)c5c4)C3)CC2)cccc1-c1ccc(N2CCc3cccc(C(=O)Nc4nc5ccccc5s4)c3C2)nc1C(=O)O] |
| 333 | 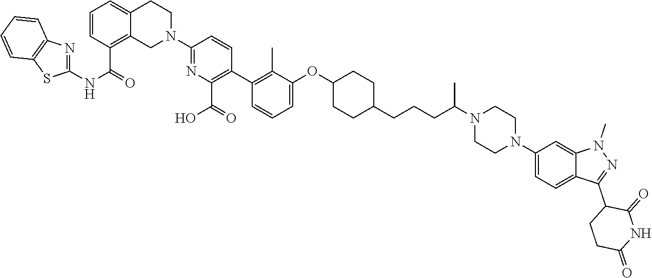 [SMILES: Cc1c(OC2CCC(CCCC(C)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| | 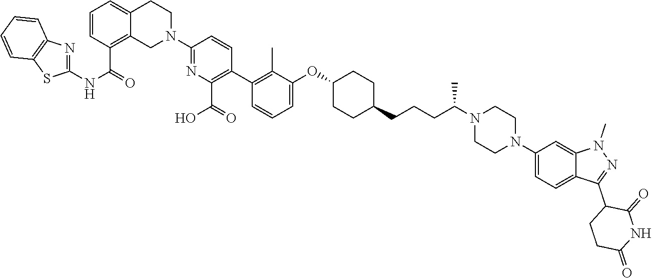 [SMILES: Cc1c(O[C@H]2CC[C@H](CCC[C@H](C)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| | 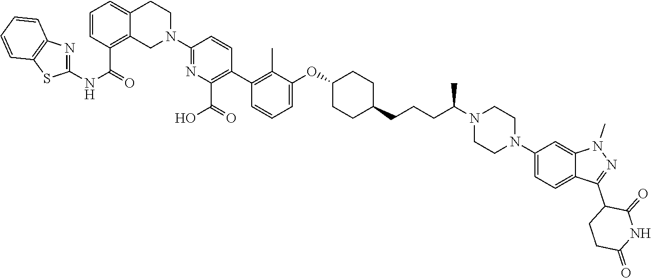 [SMILES: Cc1c(O[C@H]2CC[C@H](CCC[C@@H](C)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] |
| 349 | 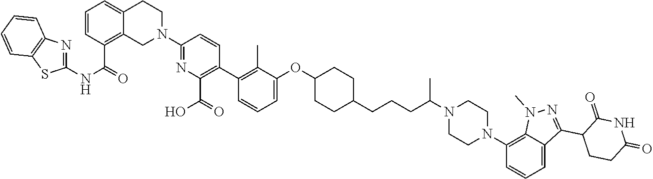 [SMILES: Cc1c(OC2CCC(CCCC(C)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| | 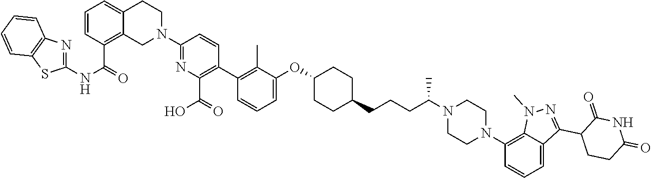 [SMILES: Cc1c(O[C@H]2CC[C@H](CCC[C@H](C)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| | 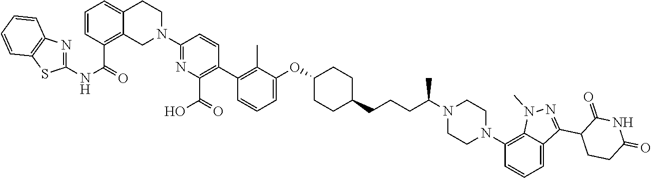 [SMILES: Cc1c(O[C@H]2CC[C@H](CCC[C@@H](C)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O] |
| 351 | 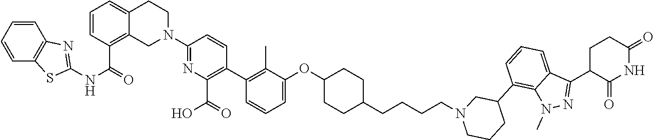 [SMILES: Cc1c(OC2CCC(CCCCN3CCCC(c4cccc5c(C6CCC(=O)NC6=O)nn(C)c45)C3)CC2)cccc1-c1ccc(N2CCc3cccc(C(=O)Nc4nc5ccccc5s4)c3C2)nc1C(=O)O] |
| | 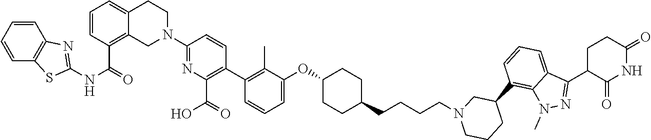 [SMILES: Cc1c(O[C@H]2CC[C@H](CCCCN3CCC[C@H](c4cccc5c(C6CCC(=O)NC6=O)nn(C)c45)C3)CC2)cccc1-c1ccc(N2CCc3cccc(C(=O)Nc4nc5ccccc5s4)c3C2)nc1C(=O)O] |
| | 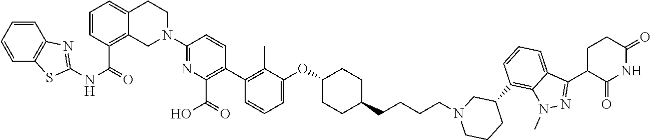 [SMILES: Cc1c(O[C@H]2CC[C@H](CCCCN3CCC[C@@H](c4cccc5c(C6CCC(=O)NC6=O)nn(C)c45)C3)CC2)cccc1-c1ccc(N2CCc3cccc(C(=O)Nc4nc5ccccc5s4)c3C2)nc1C(=O)O] |
| 352 | 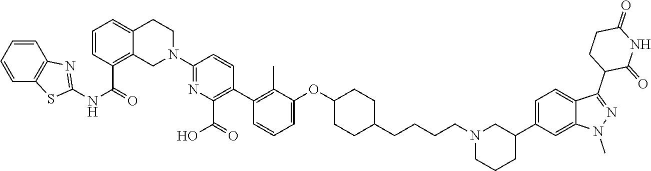 [SMILES: Cc1c(OC2CCC(CCCCN3CCCC(c4ccc5c(C6CCC(=O)NC6=O)nn(C)c5c4)C3)CC2)cccc1-c1ccc(N2CCc3cccc(C(=O)Nc4nc5ccccc5s4)c3C2)nc1C(=O)O] |
| | 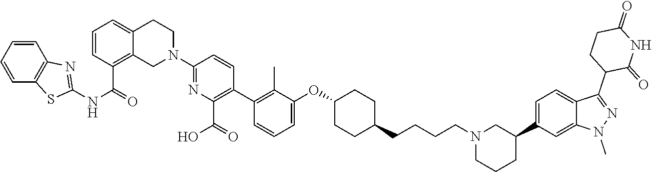 [SMILES: Cc1c(O[C@H]2CC[C@H](CCCCN3CCC[C@H](c4ccc5c(C6CCC(=O)NC6=O)nn(C)c5c4)C3)CC2)cccc1-c1ccc(N2CCc3cccc(C(=O)Nc4nc5ccccc5s4)c3C2)nc1C(=O)O] |
| | 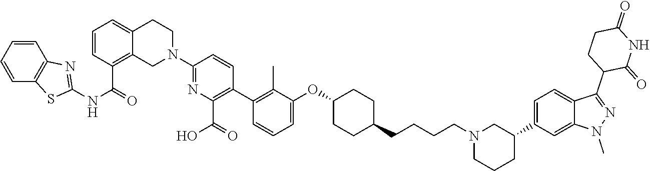 [SMILES: Cc1c(O[C@H]2CC[C@H](CCCCN3CCC[C@@H](c4ccc5c(C6CCC(=O)NC6=O)nn(C)c5c4)C3)CC2)cccc1-c1ccc(N2CCc3cccc(C(=O)Nc4nc5ccccc5s4)c3C2)nc1C(=O)O] |

In some embodiments, the compounds of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or pharmaceutically acceptable salts thereof, reduce cell viability in a cell line expressing a BCL-X_{L} protein with an EC₅₀ of less than 1 μM (e.g., less than 750 nM, less than 500 nM, or less than 200 nM). In some embodiments, the compounds reduce cell viability in a cell line expressing the BCL-X_{L} protein with an EC₅₀ of less than 200 nM (e.g., less than 150 nM, less than 200 nM, less than 100 nM, less than 10 nM, less than 1 nM). For example, the compounds can reduce cell viability in a cell line expressing the BCL-X_{L} protein with an EC₅₀ of between 0.1 nM to 100 nM, between 0.1 nM to 50 nM, between 1 nM to 50 nM, between 1 nM to 20 nM, or between 0.1 nM to 1 nM.

In some embodiments, the compounds of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or pharmaceutically acceptable salts thereof, induce degradation of a BCL-X_{L} protein in a cell line expressing the BCL-X_{L} protein with a DC₅₀ of less than 1 μM (e.g., less than 750 nM, less than 500 nM, or less than 200 nM). In some embodiments, the compounds of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, induce degradation of a BCL-X_{L} protein in a cell line expressing the BCL-X_{L} protein with a DC₅₀ of less than 200 nM (e.g., less than 150 nM, less than 200 nM, less than 100 nM, less than 10 nM, less than 1 nM). For example, the compounds can induce degradation of a BCL-X_{L} protein in a cell line expressing the BCL-X_{L} protein with a DC₅₀ of between 0.1 nM to 100 nM, between 0.1 nM to 50 nM, between 1 nM to 50 nM, between 1 nM to 20 nM, or between 0.1 nM to 1 nM.

In some embodiments, the compounds of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or pharmaceutically acceptable salts thereof, induce degradation of a BCL-X_{L} protein in a cell line expressing the BCL-X_{L} protein with a Yₘᵢₙ of less than 70% (e.g., less than 50%, less than 30%, less than 20%, or less than 10%). In some embodiments, the compounds of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, induce degradation of a BCL-X_{L} protein in a cell line expressing the BCL-X_{L} protein with a Yₘᵢₙ of less than 50% (e.g., less than 40%, less than 30%, less than 20%, less than 10%, or less than 5%). In some embodiments, the compounds of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, induce degradation of a BCL-X_{L} protein in a cell line expressing the BCL-X_{L} protein with a Yₘᵢₙ of less than 30% (e.g., less than 25%, less than 20%, less than 15%, less than 10%, or less than 5%). For example, the compounds can induce degradation of a BCL-X_{L} protein in a cell line expressing the BCL-X_{L} protein with a Yₘᵢₙ of about 1% to about 70% (e.g., about 5% to about 50% or about 10% to about 30%).

Also provided herein is a BCL-X_{L} protein non-covalently bound with a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof.

Also provided herein is a ternary complex comprising a BCL-X_{L} protein, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, and a CRBN protein, or a portion thereof.

Chemical Definitions

The term “halo” refers to fluoro (F), chloro (Cl), bromo (Br), or iodo (I).

The term “oxo” refers to a divalent doubly bonded oxygen atom (i.e., “═O”). As used herein, oxo groups are attached to carbon atoms to form carbonyls.

The term “alkyl” refers to a saturated acyclic hydrocarbon radical that may be a straight chain or branched chain, containing the indicated number of carbon atoms. For example, C₁₋₁₀ indicates that the group may have from 1 to 10 (inclusive) carbon atoms in it. Alkyl groups can either be unsubstituted or substituted with one or more substituents. Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein.

The term “haloalkyl” refers to an alkyl, in which one or more hydrogen atoms is/are replaced with an independently selected halo (e.g., —CF₃, —CHF₂, or —CH₂F).

The term “alkoxy” refers to an —O-alkyl radical (e.g., —OCH₃).

The term “alkylene” refers to a divalent alkyl (e.g., —CH₂—). Similarly, terms such as “cycloalkylene” and “heterocyclylene” refer to divalent cycloalkyl and heterocyclyl respectively. For avoidance of doubt, in “cycloalkylene” and “heterocyclylene”, the two radicals can be on the same ring carbon atom (e.g., a geminal diradical such as

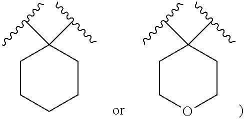 [SMILES: *C1(*)CCCCC1 ; *C1(*)CCOCC1]

or on different ring atoms (e.g., ring carbon and/or nitrogen atoms (e.g., vicinal ring carbon and/or nitrogen atoms))

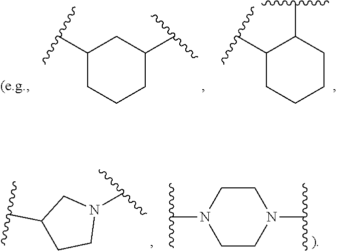 [SMILES: *C1CCCC(*)C1 ; *C1CCCCC1* ; *C1CCN(*)C1 ; *N1CCN(*)CC1]

The term “alkenyl” refers to an acyclic hydrocarbon chain that may be a straight chain or branched chain having one or more carbon-carbon double bonds. The alkenyl moiety contains the indicated number of carbon atoms. For example, C₂₋₆ indicates that the group may have from 2 to 6 (inclusive) carbon atoms in it. Alkenyl groups can either be unsubstituted or substituted with one or more substituents.

The term “alkynyl” refers to an acyclic hydrocarbon chain that may be a straight chain or branched chain having one or more carbon-carbon triple bonds. The alkynyl moiety contains the indicated number of carbon atoms. For example, C₂₋₆ indicates that the group may have from 2 to 6 (inclusive) carbon atoms in it. Alkynyl groups can either be unsubstituted or substituted with one or more substituents.

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system); and wherein 0, 1, 2, 3, or 4 atoms of each ring may be substituted by a substituent. Examples of aryl groups include phenyl, naphthyl, tetrahydronaphthyl, and the like.

The term “cycloalkyl” as used herein refers to mono-, bi-, tri-, or polycyclic (e.g., fused, bridged, or spirocyclic bi-, tri-, or polycyclic) saturated or partially unsaturated hydrocarbon groups having, e.g., 3 to 20 ring carbons, preferably 3 to 15 ring carbons, and more preferably 3 to 12 ring carbons or 3-10 ring carbons or 3-6 ring carbons, wherein the cycloalkyl group may be optionally substituted. The term “saturated” as used in this context means only single bonds present between constituent carbon atoms. Examples of saturated cycloalkyl groups include, without limitation, cyclopropyl, cyclobutyl, cyclopentyl, cyclohexyl, cycloheptyl, and cyclooctyl. Partially unsaturated cycloalkyl may have any degree of unsaturation provided that one or more double bonds is present in the cycloalkyl, none of the rings in the ring system are aromatic, and the partially unsaturated cycloalkyl group is not fully saturated overall. Examples of partially unsaturated cycloalkyl include, without limitation, cyclopentenyl, cyclohexenyl, cycloheptenyl, and cyclooctenyl. Non-limiting examples of fused/bridged cycloalkyl includes: bicyclo[1.1.0]butyl, bicyclo[2.1.0]pentyl, bicyclo[1.1.1]pentyl, bicyclo[3.1.0]hexyl, bicyclo[2.1.1]hexyl, bicyclo[3.2.0]heptyl, bicyclo[4.1.0]heptyl, bicyclo[2.2.1]heptyl, bicyclo[3.1.1]heptyl, bicyclo[4.2.0]octyl, bicyclo[3.2.1]octyl, bicyclo[2.2.2]octyl, and the like. Non-limiting examples of spirocyclic cycloalkyls include spiro[2.2]pentyl, spiro[2.5]octyl, spiro[3.5]nonyl, spiro[3.5]nonyl, spiro[3.5]nonyl, spiro[4.4]nonyl, spiro[2.6]nonyl, spiro[4.5]decyl, spiro[3.6]decyl, spiro[5.5]undecyl, and the like.

The term “heteroaryl”, as used herein, means a mono-, bi-, tri- or polycyclic group having 5 to 20 ring atoms, alternatively 5, 6, 9, 10, or 15 ring atoms; wherein at least one ring in the system contains one or more heteroatoms independently selected from the group consisting of N, O, S (inclusive of oxidized forms such as:

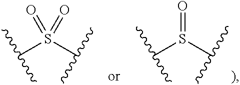

and P (inclusive of oxidized forms such as:

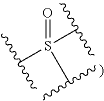

(e.g., N, O, and S (inclusive of oxidized forms such as:

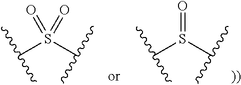

and at least one ring in the system is aromatic (but does not have to be a ring which contains a heteroatom, e.g. tetrahydroisoquinolinyl, e.g., tetrahydroquinolinyl). In some embodiments, heteroaryl groups contain 1-4 (e.g., 1, 2, or 3) ring heteroatoms each independently selected from the group consisting of N, O, and S (inclusive of oxidized forms such as:

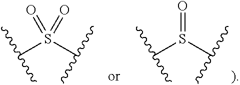

Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridinyl, pyrazolo[4,3-b]pyridinyl, tetrazolyl, chromanyl, 2,3-dihydrobenzo[b][1,4]dioxinyl, benzo[d][1,3]dioxolyl, 2,3-dihydrobenzofuranyl, tetrahydroquinolinyl, 2,3-dihydrobenzo[b][1,4]oxathiinyl, isoindolinyl, and others. In some embodiments, the heteroaryl is selected from thienyl, pyridinyl, furyl, pyrazolyl, imidazolyl, isoindolinyl, pyranyl, pyrazinyl, and pyrimidinyl. For purposes of clarification, heteroaryl also includes aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of pyridone

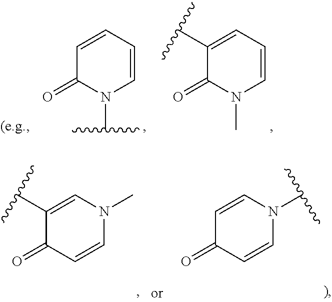

pyrimidone

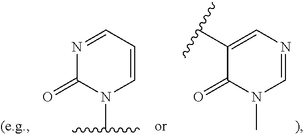

pyridazinone

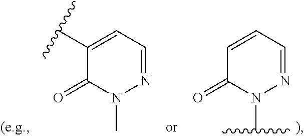

pyrazinone

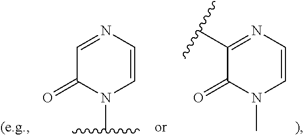

and imidazolone

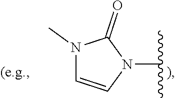 [SMILES: *n1ccn(C)c1=O]

wherein each ring nitrogen adjacent to a carbonyl is tertiary (i.e., the oxo group (i.e., “═O”) herein is a constituent part of the heteroaryl ring).

The term “heterocyclyl” refers to a mono-, bi-, tri-, or polycyclic (e.g., fused, bridged, or spirocyclic bi-, tri-, or polycyclic) saturated or partially unsaturated ring system with 3-15 ring atoms (e.g., 5-8 membered monocyclic, 8-12 membered bicyclic, or 11-15 membered tricyclic ring system) having 1-3 heteroatoms if monocyclic, 1-6 heteroatoms if bicyclic, or 1-9 heteroatoms if tricyclic or polycyclic, said heteroatoms selected from O, N, S (inclusive of oxidized forms such as:

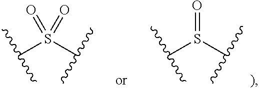

and P (inclusive of oxidized forms such as:

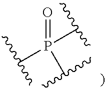 [SMILES: *P(*)(*)=O]

(e.g., O, N, and S (inclusive of oxidized forms such as:

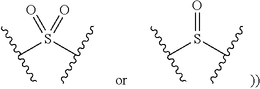

(e.g., carbon atoms and 1-3, 1-6, or 1-9 heteroatoms of N, O, S, or P if monocyclic, bicyclic, or tricyclic, respectively), wherein 0, 1, 2 or 3 atoms of each ring may be substituted by a substituent. In some embodiments, heterocyclyl groups contain 1-4 (e.g., 1, 2, or 3) ring heteroatoms each independently selected from the group consisting of N, O, and S (inclusive of oxidized forms such as:

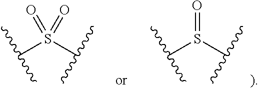

The term “saturated” as used in this context means only single bonds present between constituent ring atoms and other available valences occupied by hydrogen and/or other substituents as defined herein. Examples of saturated heterocyclyl groups include piperazinyl, pyrrolidinyl, dioxanyl, morpholinyl, tetrahydrofuranyl, and the like. Partially unsaturated heterocyclyl groups may have any degree of unsaturation provided that one or more double bonds is present in the heterocyclyl, none of the rings in the ring system are aromatic, and the partially unsaturated heterocyclyl group is not fully saturated overall. Examples of partially unsaturated heterocyclyl groups include, without limitation, tetrahydropyridyl, dihydropyrazinyl, dihydropyridyl, dihydropyrrolyl, dihydrofuranyl, dihydrothiophenyl. Non-limiting examples of fused/bridged heterocyclyl includes: 2-azabicyclo[1.1.0]butyl, 2-azabicyclo[2.1.0]pentyl, 2-azabicyclo[1.1.1]pentyl, 3-azabicyclo[3.1.0]hexyl, 5-azabicyclo[2.1.1]hexyl, 3-azabicyclo[3.2.0]heptyl, octahydrocyclopenta[c]pyrrole, 3-azabicyclo[4.1.0]heptyl, 7-azabicyclo[2.2.1]heptyl, 6-azabicyclo[3.1.1]heptyl, 7-azabicyclo[4.2.0]octyl, 2-azabicyclo[2.2.2]octyl, 3-azabicyclo[3.2.1]octyl, 2-oxabicyclo[1.1.0]butyl, 2-oxabicyclo[2.1.0]pentyl, 2-oxabicyclo[1.1.1]pentyl, 3-oxabicyclo[3.1.0]hexyl, 5-oxabicyclo[2.1.1]hexyl, 3-oxabicyclo[3.2.0]heptyl, 3-oxabicyclo[4.1.0]heptyl, 7-oxabicyclo[2.2.1]heptyl, 6-oxabicyclo[3.1.1]heptyl, 7-oxabicyclo[4.2.0]octyl, 2-oxabicyclo[2.2.2]octyl, 3-oxabicyclo[3.2.1]octyl, and the like. Non-limiting examples of spirocyclic heterocyclyls include 2-azaspiro[2.2]pentyl, 4-azaspiro[2.5]octyl, 1-azaspiro[3.5]nonyl, 2-azaspiro[3.5]nonyl, 7-azaspiro[3.5]nonyl, 2-azaspiro[4.4]nonyl, 6-azaspiro[2.6]nonyl, 1,7-diazaspiro[4.5]decyl, 7-azaspiro[4.5]decyl 2,5-diazaspiro[3.6]decyl, 3-azaspiro[5.5]undecyl, 2-oxaspiro[2.2]pentyl, 4-oxaspiro[2.5]octyl, 1-oxaspiro[3.5]nonyl, 2-oxaspiro[3.5]nonyl, 7-oxaspiro[3.5]nonyl, 2-oxaspiro[4.4]nonyl, 6-oxaspiro[2.6]nonyl, 1,7-dioxaspiro[4.5]decyl, 2,5-dioxaspiro[3.6]decyl, 1-oxaspiro[5.5]undecyl, 3-oxaspiro[5.5]undecyl, 3-oxa-9-azaspiro[5.5]undecyl and the like.

A nitrogen-containing heterocyclyl as used herein refers to a heterocyclyl having 1-2 ring nitrogen atoms and 0-2 additional ring heteroatoms selected from the group consisting of O and S (inclusive of oxidized such as:

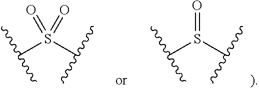

The nitrogen-containing heterocyclyl can be monocyclic, bicyclic, or polycyclic as defined elsewhere herein. Examples of monocyclic nitrogen-containing heterocyclyl include azetidinyl, pyrrolidinyl, piperidinyl, piperazinyl, morpholinyl, and the like. Examples of bicyclic nitrogen-containing heterocyclyl include 7-azaspiro[3.5]nonyl, 1,7-diazaspiro[4.5]decyl, 3-oxa-7,9-diazabicyclo[3.3.1]nonanyl, 2,6-diazaspiro[3.3]heptanyl, and the like.

As used herein, when a ring is described as being “partially unsaturated”, it means said ring has one or more additional degrees of unsaturation (in addition to the degree of unsaturation attributed to the ring itself; e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

For the avoidance of doubt, and unless otherwise specified, for rings and cyclic groups (e.g., aryl, heteroaryl, heterocyclyl, heterocycloalkenyl, cycloalkenyl, cycloalkyl, and the like described herein) containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge

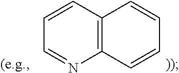

(ii) a single ring atom (spiro-fused ring systems)

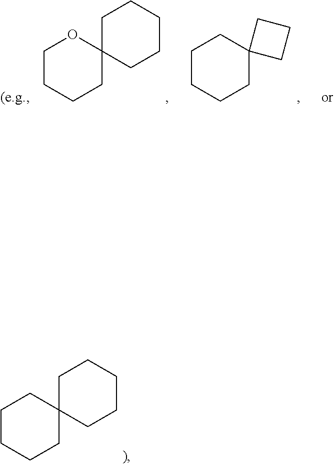

or (iii) a contiguous array of ring atoms (bridged ring systems having all bridge lengths>0)

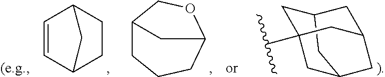

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

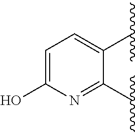 [SMILES: *c1ccc(O)nc1*]

encompasses the tautomeric form containing the moiety:

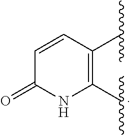 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

The compounds provided herein may encompass various stereochemical forms. The compounds also encompass diastereomers as well as optical isomers, e.g., mixtures of enantiomers including racemic mixtures, as well as individual enantiomers and diastereomers, which arise as a consequence of structural asymmetry in certain compounds. Unless otherwise indicated, when a disclosed compound is named or depicted by a structure without specifying the stereochemistry and has one or more chiral centers, it is understood to represent all possible stereoisomers of the compound.

Methods of Treatment

Indications

Provided herein are methods for inducing degradation of a BCL-X_{L} protein. For example, provided herein are compounds capable of inducing degradation of a BCL-X_{L} protein useful for treating or preventing cancers. See, e.g., Guo, et al. Aging (Albany NY) 13.15 (2021): 19750; Park, et al. Proceedings of the National Academy of Sciences 112.40 (2015): 12492-12497; Zhang, et al. Molecular Cancer 14.1 (2015): 1-9; Beroukhim, et al. Nature 463.7283 (2010): 899-905. Additional methods of evaluating the binding of a compound to BCL-X_{L} protein or the inhibition of a BCL-X_{L} protein are described in, for example, U.S. Patent Publication Nos. 2007/027135; 2010/305122, and 2013/096120.

It will be understood that the effect of protein degradation typically increases over time, though the appearance of degradation (e.g., as expressed by the percentage degradation compared to a control, or the parameters Yₘᵢₙ, DC₅₀, and/or Dₘₐₓ) is affected by the resynthesis rate of the protein. It is common in the art to examine degradation after a specified period of time, such as 6 hours, 12 hours, 18 hours, 1 day, 2 days, 3 days, or more. For example, degradation can be expressed as the percent degradation after 24 hours.

Exemplary assays for validating the degradation-inducing mechanism of a compound as provided herein are known in the art and are described, for example, in International Publication No. WO 2019/144117 and Wu, et al. Nature Structural & Molecular Biology 27.7 (2020): 605-614.

Degradation assays can be used to quantify both on- and off-target degradation-inducing effects of compounds, such as those provided herein. Exemplary assays include, quantitative immunoblotting, other immunoassays (e.g., MesoScale Discovery (MSD) immunoassays), homogenous time resolved florescence (HTRF), and HiBiT. In some embodiments, cells can be contacted with a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, incubated, and then the lysate can be prepared for gel electrophoresis (e.g., SDS-PAGE), followed by immunoblotting and quantification compared to a control (e.g., a DMSO-treated control). As another example, a cell line can be engineered to express a HiBiT-tagged BCL-X_{L} protein, and the amount of fluorescence observed when the complementary LgBiT peptide is added can be compared between cells treated with a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, and a control (e.g., a DMSO-treated control). See, for instance, Example B1 and Example B2. See also, e.g., International Publication Nos. WO 2020/163823 and WO 2019/144117. In some embodiments, off-target degradation inducing effects can be assessed for the proteins Eukaryotic peptide chain release factor GTP-binding subunit ERF3A (GSPT1), Ikaros (IKZF1), Helios (IKZF2), Aiolos (IKZF3), and/or casein kinase I isoform alpha (CK1α).

See also the assays described in International Publication Nos. WO 2023/044046; WO 2022/169780; WO 2021/222114; WO 2021/146536; WO 2021/078301; WO 2021/007307; WO 2020/163823; WO 2019/144117; WO 2017/184995, and Khan, et al. Nature Medicine 25.12 (2019): 1938-1947; Balachander, et al. Clinical Cancer Research 26.24 (2020): 6535-6549.

Binding affinity of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, as provided herein to BCL-X_{L} can be determined by, for example, a binding IC₅₀ or Kᵢ value (e.g., using a competition assay), or by a K_{D} value (e.g., using a biophysical assay). A compound with a lower binding IC₅₀ value, as determined under substantially similar conditions, is a more potent binder relative to a compound with a higher binding IC₅₀ value. A compound with a lower binding Kᵢ value, as determined under substantially similar conditions, is a more potent binder relative to a compound with a higher binding Kᵢ value. Similarly, a compound with a lower K_{D} value, as determined under substantially similar conditions, is a more potent binder relative to a compound with a higher K_{D} value. For example, a binding IC₅₀ value can be determined in a fluorescence polarization assay using a fluorescently labeled BH3-only peptide (e.g., BAD or BAX) as the competitor. As another example, a binding Kᵢ value can be determined using a time resolved-fluorescence resonance energy transfer (TR-FRET) assay using a fluorescently labeled BH3-only peptide (e.g., BAK) and a fluorescently labeled antibody that binds to BCL-X_{L}, where the fluorophores are a FRET pair, and using a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, as provided herein as a competitor for the BH3-only peptide. See, e.g., U.S. Patent Publication Nos. 2007/0027135, 2010/305122, and 2013/096120.

The ability of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (TI) (e.g., (II-a)), or a pharmaceutically acceptable salt thereof, as provided herein to inhibit BCL-X_{L} can be determined using an IC₅₀ value. A compound with a lower IC₅₀ value, as determined under substantially similar conditions, is a more potent inhibitor relative to a compound with a higher IC₅₀ value. One way that inhibition of BCL-X_{L} can be measured is measuring the interruption of the formation of a complex of BCL-X_{L} with a BH3-only peptide (e.g., BIM). For example, an electrochemiluminescence-based sandwich ELISA assay (e.g., a Meso Scale Discovery (MSD)-ELISA assay) can be used. See, for example, Phillips, D. C., et al. Blood Cancer Journal 5.11 (2015): e368-e368 and Xiao, Yu, et al. Molecular Cancer Therapeutics 14.8 (2015): 1837-1847. In such assays, cells expressing BCL-X_{L} can be incubated with a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)) for a period of time, lysed, and then evaluated in the assay. A tagged anti-BCL-X_{L} antibody (e.g., a biotin-tagged anti-BCL-X_{L} antibody) can be immobilized on an assay plate (e.g., a streptavidin assay plate), and then the lysate can be applied to pull down BCL-X_{L}. An anti-BIM antibody (e.g., a rabbit anti-BIM antibody) can be introduced, followed by addition of a detection antibody (e.g., a sulfo-tagged goat anti-rabbit antibody) and measurement of the detection antibody. As another example, interruption of the formation of a complex of BCL-X_{L} with a BH3-only peptide (e.g., BIM) can be measured using a mammalian two-hybrid assay. In such assays, a plasmid encoding the ‘bait’ and ‘prey’ fusion proteins (e.g., the DNA binding domain of GAL4 fused to BCL-X_{L} and the transcriptional activation domain of VP16 fused to BIM) can be introduced to cells (e.g., HeLa cells) stably expressing a GAL4-luciferase reporter. A compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)) can be added to the cells in culture, incubated, and the luciferase activity can be measured. See, for example, Souers, Andrew J., et al. Nature Medicine 19.2 (2013): 202-208.

Potency of degradation by a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, as provided herein can be determined by DC₅₀ value. As used herein, DC₅₀ refers to the concentration of the compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)) that results in a 50% decrease in the concentration of a protein (e.g., BCL-X_{L} protein) in a cell compared to the concentration of the protein before the cell is contacted with the compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or compared to the concentration of the protein in a cell not contacted with the compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)). A compound with a lower DC₅₀ value, as determined under substantially similar conditions, is a more efficient inducer of degradation relative to a compound with a higher DC₅₀ value. In some embodiments, a DC₅₀ value can be determined (e.g., using HiBiT detection) in vitro or in vivo (e.g., in tumor cells (e.g., cell lines such as MOLT4, RS4;11, NCI-H146, EJM, HEK293T, HT1080, and/or H929) expressing a BCL-X_{L} protein).

Potency of degradation by a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, as provided herein can be determined by EC₅₀ value. As used herein, EC₅₀ refers to the concentration of the compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)) that results in a 50% decrease in the concentration of a protein (e.g., BCL-X_{L} protein) relative to the trough concentration of the protein in a cell, when compared to the concentration of the protein before the cell is contacted with the compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or compared to the concentration of the protein in a cell not contacted with the compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)). A compound with a lower EC₅₀ value, as determined under substantially similar conditions, is a more potent compound relative to a compound with a higher EC₅₀ value. In some embodiments, an EC₅₀ value can be determined (e.g., using HiBiT detection) in vitro or in vivo (e.g., in tumor cells (e.g., cell lines such as MOLT4, RS4;11, NCI-H146, EJM, HEK293T, HT1080, and/or H929) expressing a BCL-X_{L} protein).

Potency of degradation by a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, as provided herein can be determined by Yₘᵢₙ value. As used herein, Yₘᵢₙ refers to the ratio of trough concentration of a protein (e.g., BCL-X_{L} protein) in a cell compared to the concentration of the protein before the cell is contacted with the compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or compared to the concentration of the protein in a cell not contacted with the compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), expressed as a percentage. As used herein, Dₘₐₓ is 1-Yₘᵢₙ. Yₘᵢₙ can be measured by a HiBiT assay as described in Example B1. A compound with a lower Yₘᵢₙ value, as determined under substantially similar conditions, is a more potent inducer of degradation relative to a compound with a higher Yₘᵢₙ value. A compound with a lower Yₘᵢₙ value, as determined under substantially similar conditions, is a more potent compound relative to a compound with a higher Yₘᵢₙ value. In some embodiments, a Yₘᵢₙ value can be determined (e.g., using HiBiT detection) in vitro or in vivo (e.g., in tumor cells (e.g., cell lines such as MOLT4, RS4;11, NCI-H146, EJM, HEK293T, HT1080, and/or H929) expressing a BCL-X_{L} protein).

An exemplary assay for determining the potency of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, includes measuring the effect of the compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, on cell proliferation and/or viability. Cell proliferation assays can be performed in a number of formats, including 2D and 3D. Similarly, a cell proliferation assay can be performed with any appropriate cell line, including, for example, MOLT4, RS4;11, NCI-H146, EJM, HEK293T, HT1080, and/or H929. As an illustrative example, a 3D cell proliferation assay can include growing cells in a 3D medium, contacting the cells with a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, measuring the cellular proliferation using an appropriate reagent (e.g., CELLTITERGLO® 3D), and then comparing the signal from an experiment with a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, to the signal from a control experiment (e.g., lacking the compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof). As another illustrative example, a 2D cell proliferation assay can include plating cells onto a growth surface, optionally letting the cells grow for a period of time, contacting the cells with a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, measuring the cellular proliferation using an appropriate reagent (e.g., CELLTITERGLO®), and then comparing the signal from an experiment with compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, to the signal from a control experiment (e.g., lacking a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof). Additional cell viability assays include MTT assays, which are colorimetric assays based on the reduction of the tetrazolium dye MTT (3-(4,5-dimethylthiazol-2-yl)-2,5-diphenyltetrazolium bromide) to the insoluble purple formazan, and other similar assays based on related tetrazolium salts. See, for instance, Example B3 and Example B4.

A cell viability assay can be used to measure the effect of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, on cell death. For example, cells expressing BCL-X_{L} protein (e.g., MOLT-4 cells) can be incubated with various concentrations of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-E b-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, then exposed to a detection reagent (e.g., a CELLTITER-GLO® Cell Viability Assay kit) to determine cell viability. An exemplary assay for evaluating the affinity of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, includes using a competition assay with recombinant BCL-X_{L} protein. For example, purified recombinant affinity-tagged (e.g., His-tagged) BCL-X_{L} protein can be incubated with various concentrations of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I—F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, and a fixed concentration of affinity-tagged (e.g., biotin-tagged) BAD protein. After a period of incubation, FRET acceptor beads with a complementary affinity tag (e.g., His-acceptor beads) and FRET donor beads (e.g., streptavidin-tagged donor beads) can be added to the mixture, and a FRET reaction can be used to determine an inhibition constant of the compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or pharmaceutically acceptable salt thereof. For example, an AlphaLISA competitive assay can be performed. See, e.g., International Publication No. WO 2019/144117.

An exemplary assay for determining the mechanism of cell death using of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, includes measuring the effect of the compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, on one or more markers of a mechanism of cell death (e.g., apoptosis). Exemplary markers of apoptosis include caspase induction (e.g., caspase 3/7 induction) and annexin V staining. Such assays may also be used as determinants of cell viability. For example, cells expressing BCL-X_{L} protein (e.g., MOLT-4 cells) can be incubated with various concentrations of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, and relative caspase activity can be assessed using a luciferase substrate that is activated by caspase 3/7 (e.g., using the CASPASE-GLO® 3/7 assay). As another example, cells expressing BCL-X_{L} protein (e.g., MOLT-4 cells) can be incubated with various concentrations of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, and relative caspase activity can be assessed using a dye that is coupled to an activated caspase motif (e.g., INCUCYTE® Caspase 3/7 Green Apoptosis Assay Reagent), followed by analysis using a platform for live cell imaging (e.g., an INCUCYTE® SX5 Live-Cell Analysis Instrument). As another example, cells expressing BCL-X_{L} protein (e.g., MOLT-4 cells) can be incubated with various concentrations of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (I-a)), or a pharmaceutically acceptable salt thereof, and annexin V positivity can be assessed using phosphatidylserine dye (e.g., an INCUCYTE® Annexin V dye), followed by analysis using a platform for live cell imaging (e.g., an INCUCYTE® SX5 Live-Cell Analysis Instrument). See, for instance, Example B5.

As another example, the potency and/or efficacy of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, can be evaluated in an animal model, for example, a xenograft model (e.g., using an established cancer cell line such as MOLT4, HEL, TF1, F36P, OCI-M1, OCI-M2, SET-2, CMK, M07E, or UKE-1, or a patient-derived xenograft (PDX) model). For example, a PDX model can be run in immunodeficient mice (e.g., athymic nude, outbred homozygous (e.g., Crl:NU(NCr)—Foxn1^{nu}) or Fox Chase SCID (CB17/Icr-Prkdc^{scid}/IcrIcoCrl), mice). The mice can be female, 6-12 weeks old at tumor implantation and have access to food and water ad libitum. Approximately 70 mg of a tumor can be implanted subcutaneously in the right flank of each mouse. Following implantation, tumors can be measured weekly and once the tumor volumes reach 150-300 mm³, the mice can be randomized into treatment and control groups. In some embodiments, one or more experimental arms can be added to evaluate pharmacokinetics and/or pharmacodynamics. The mice can be treated with a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, (e.g., via IP or PO (oral) administration) and optionally an additional therapy or therapeutic agent (e.g., any of the additional therapies or therapeutic agents described herein). Throughout the study, health condition, body weight and tumor volumes of the mice can be recorded on a weekly basis. The mice can be sacrificed at 28 days or when the tumor reaches 1 cm³, and the tumors can be evaluated (e.g., by tumor weight, by tumor volume). At the end of each study, the Best Response can be calculated for each treatment arm. Best Response is defined as the minimum value of ΔVolumeₜ for t≥10 days. Best Responses between the control arm(s) and the treatment arm(s) can be compared to determine if the treatment(s) work better than the control(s). In some embodiments, tumor samples can also be collected at the end of each study and relevant proteins (e.g., BCL-X_{L}, BCL-2, MCL-1, BIM, BAX, and/or BAK) can be measured to determine if the treatment might have a better protein modulation profile compared to a control. In some embodiments, tumor samples can also be collected at the end of each study and analyzed for signaling pathway activity (e.g., via phosphoERK levels). For pharmacokinetic and pharmacodynamic studies, tumor and/or blood samples from the mice can be obtained at the same or different time points than efficacy studies. For example, for pharmacokinetic and pharmacodynamic studies, tumor and/or blood samples from the mice can be obtained at Day 5, 6 hours post dosing, and relevant proteins can be measured in the tumor samples and pharmacokinetic studies can be performed on the blood samples or a portion thereof (e.g., plasma).

In some embodiments, the PDX is a model of a myeloproliferative neoplasm (MPN) (e.g., CEL, CML, CNL, essential thrombocythemia (e.g., JAK2 mutant (e.g., JAK2 V617F mutant) essential thrombocythemia or JAK2 wild type essential thrombocythemia), polycythemia vera (e.g., JAK2 mutant (e.g., JAK2 V617F mutant) polycythemia vera or JAK2 wild type polycythemia vera), or myelofibrosis (e.g., primary myelofibrosis (e.g., JAK2 mutant (e.g., JAK2 V617F mutant) primary myelofibrosis or JAK2 wild type primary myelofibrosis), post-essential thrombocythemia myelofibrosis (e.g., JAK2 mutant (e.g., JAK2 V617F mutant) post-essential thrombocythemia myelofibrosis or JAK2 wild type post-essential thrombocythemia myelofibrosis), or post-polycythemia vera myelofibrosis (e.g., JAK2 mutant (e.g., JAK2 V617F mutant) post-polycythemia vera myelofibrosis or JAK2 wild type post-polycythemia vera myelofibrosis)))), a CRC (e.g., BRaf mutant CRC (e.g., Braf V600E CRC) or KRas mutant CRC (e.g., KRas G12C mutant CRC or KRas G12D CRC)), SCLC (e.g., ASCL1 subtype SCLC or NEUROD1 subtype SCLC), a NSCLC (e.g., BRaf mutant NSCLC (e.g., Braf V600E NSCLC), an EGFR mutant NSCLC (e.g., EGFR L858R NSCLC or EGFR exon 19 deletion NSCLC), MET mutant NSCLC (e.g., MET exon 14 deletion NSCLC, MET amplified NSCLC), a KRas mutant NSCLC (e.g., KRas G12C NSCLC)), a lung squamous cell carcinoma, a malignant pleural mesothelioma (e.g., a BAP1 mutant malignant pleural mesothelioma), a melanoma (e.g., Braf mutant melanoma (e.g., Braf V600E melanoma)), a breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), a lymphoma (e.g., a T cell lymphoma (e.g., anaplastic large T cell lymphoma, cutaneous T cell lymphoma, or peripheral T cell lymphoma), or a non-Hodgkin lymphoma (e.g., DLBCL, anaplastic large T cell lymphoma, cutaneous T cell lymphoma, or peripheral T cell lymphoma), a leukemia (e.g., T cell leukemia (e.g., a T-ALL (e.g., relapsed/refractory T-ALL)), post-MPN leukemia, M6-AML, M7-AML), a head and neck cancer, a pancreatic cancer, a bladder cancer, an ovarian cancer (e.g., BRCA1 mutant ovarian cancer or BRCA2 mutant ovarian cancer, HGSOC (e.g., BRCA1 mutant HGSOC or BRCA2 mutant HGSOC)), a cervical cancer, an intrahepatic cholangiocarcinoma, or a mesenchymal cancer (e.g., mesenchymal breast cancer or mesenchymal kidney cancer).

See, e.g., Khan, et al. Nature Medicine 25.12 (2019): 1938-1947; Balachander, et al. Clinical Cancer Research 26.24 (2020): 6535-6549.

The pharmacokinetic parameters of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, can be evaluated in an animal model, for instance, a mouse model, a rat model, a dog model, or a nonhuman primate (e.g., cynomolgus monkey) model. An exemplary assay includes the following. For example, pharmacokinetics (PK) studies can be conducted on animals (e.g., male or female CD-1 mice, Sprague Dawley rats, beagle dogs, or cynomolgus monkeys) by two delivery routes: intravenous (IV) injection and oral gavage (PO). Animals in both the IV and PO groups (e.g., n=3) are allowed free access to food and water. A compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, can be formulated in solution for the IV route and solution or suspension for the PO route. On the day of the experiment, the compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, can be administered via vein injection (e.g., at 1 mg/kg) for IV route or via oral gavage (e.g., at 5 to 10 mg/kg) for PO route. In some cases, the animals can be orally pre-dosed with a cytochrome P450 inhibitor (e.g., 1-aminobenzotriazole) prior to (e.g., 16 hours prior to) dosing the compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof. Blood samples can be collected via serial bleeding (e.g., at 8 timepoints from 0.83 to 24 hours post dose). At each timepoint, blood can be collected (e.g., approximately 30 μL of blood/timepoint) in a K₂EDTA tube via a vein (e.g., the saphenous vein). Blood samples can be put on wet ice and centrifuged (e.g., at 4600 RPM for 4 minutes) to obtain plasma samples. Plasma samples can be diluted (e.g., with an equal volume of pH 3.0 phosphate buffer) and submitted to LC-MS/MS for sample analysis. Pharmacokinetic parameters, including clearance (IV or PO, depending on the mode of dosing), area under the curve (AUC), and oral bioavailability (% F) can be calculated using a non-compartmental model.

In some embodiments, the % F for a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, is at least 4%. In some embodiments, the % F for a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, is at least 10%. In some embodiments, the % F for a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, is at least 20%. In some embodiments, the % F for a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, is at least 30%. In some embodiments, the % F for a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, is at least 40%. In some embodiments, the % F for a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, is about 4% to about 80% (e.g., about 4% to about 60%, about 4% to about 40%, about 4% to about 20%, about 4% to about 10%, about 20% to about 40%, or about 20% to about 30%). In some embodiments, the % F for a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, is about 4% to about 20%. In some embodiments, the % F for a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, is about 20% to about 40%. In some embodiments, the % F for a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, is about 40% to about 60%. In some embodiments, the % F for a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, is about 60% to about 80%.

In some embodiments, the clearance for a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, dosed PO in rats at 10 mg/kg, is less than 10 mL/min/kg (e.g., less than 5 mL/min/kg, less than 3 mL/min/kg, or less than 1 mL/min/kg). In some embodiments, clearance for a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, dosed PO in rats at 10 mg/kg, is about 0.05 mL/min/kg to about 5 mL/min/kg (e.g., about 0.05 mL/min/kg to about 3 mL/min/kg, about 0.05 mL/min/kg to about 1 mL/min/kg, or about 0.05 mL/min/kg to about 0.5 mL/min/kg).

In some embodiments, the AUC for a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, dosed PO in rats at 10 mg/kg, is about 10 μM·h to about 150 μM·h (e.g., about 10 μM·h to about 100 μM·h, about 10 μM·h to about 50 μM·h, or about 30 μM·h to about 80 μM·h).

Heterobifunctional degraders can, in some cases, induce the degradation of off-target proteins. For heterobifunctional degraders that utilize CRBN, common off-target proteins that can be degraded include GSPT1, IKZF1, IKZF2, IKZF3, and/or CK1α. This degradation is generally believed to be due to the E3 binding moiety of the heterobifunctional degrader facilitating ternary complex formation between the off-target protein and CRBN. GSPT1 is a translation termination factor, and CK1α is a kinase that is involved in many key cellular processes including cell cycle progression and chromosome segregation; these are both commonly essential genes, so undesired degradation of either or both may lead to nonspecific cytotoxicity. The IKZF proteins are zinc finger transcription factors that are involved with cell fate during hematopoiesis, and degradation of these proteins has been associated with hematotoxicity. See, e.g., Moreau, Kevin, et al. British Journal of Pharmacology 177.8 (2020): 1709-1718.

In some embodiments, the compounds of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or pharmaceutically acceptable salts thereof, can exhibit potent and selective induction of degradation of a BCL-X_{L} protein. In some embodiments, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, or a pharmaceutically acceptable salt thereof, can selectively target a BCL-X_{L} protein for degradation over an off-target protein, such as another BCL-2 family member (e.g., BCL-2 and/or MCL-1) or non-BCL-2 family member target (e.g., GSPT1, IKZF1, IKZF2, IKZF3, and/or CK1α).

As used herein, “selective” or “selectively”, when referring to a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof in a protein degradation assay, indicates at least a 5-fold (e.g., at least a 10-fold, at least a 25-fold, at least a 50-fold, or at least a 100-fold) superior performance in the protein degradation assay for a specified protein with reference to a comparator protein in the assay. In some embodiments, the specified protein is BCL-X_{L} protein and the comparator is BCL-2 protein. For example, if a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, “selectively” induces degradation of BCL-X_{L} protein over BCL-2 protein as determined by a degradation assay, then the compound has at least a 5-fold (e.g., at least a 10-fold, at least a 25-fold, at least a 50-fold, or at least a 100-fold) smaller DC₅₀ value for BCL-X_{L} protein than for the BCL-2 protein when measured by the degradation assay.

In some embodiments, the compounds provided herein can exhibit potency (e.g., nanomolar potency) against a BCL-X_{L} protein with minimal activity (e.g., micromolar potency) against BCL-2 family members (e.g., BCL-2 or MCL-1 proteins). In some embodiments, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, can exhibit potent degradation of a BCL-X_{L} protein and have minimal potency in degrading (e.g., as measured by Yₘᵢₙ, DC₅₀, and/or Dₘₐₓ values) an off-target protein (e.g., a BCL-2 family member (e.g., BCL-2 and/or MCL-1), GSPT1, IKZF1, IKZF2, IKZF3 and/or CK1α). In some embodiments, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, can exhibit greater induction of degradation of a BCL-X_{L} protein relative to induction of degradation (e.g., as measured by Yₘᵢₙ, DC₅₀, and/or Dₘₐₓ values) of an off-target protein (e.g., a BCL-2 family member (e.g., BCL-2 and/or MCL-1), GSPT1, IKZF1, IKZF2, IKZF3, and/or CK1α). In some embodiments, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, can exhibit at least 2-fold, 3-fold, 5-fold, 10-fold, 25-fold, 50-fold, or 100-fold greater induction of degradation of a BCL-X_{L} protein relative to induction of degradation of an off-target protein. In some embodiments, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, can exhibit up to 1000-fold greater induction of degradation of a BCL-X_{L} protein relative to induction of degradation of an off-target protein. In some embodiments, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, can exhibit from about 2-fold to about 10-fold greater induction of degradation of a BCL-X_{L} protein relative to induction of degradation of an off-target protein (e.g., GSPT1, IKZF1, IKZF2, and/or IKZF3 and/or CK1a) (e.g., as measured by Yₘᵢₙ, DC₅₀, and/or Dₘₐₓ values). In some embodiments, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, can exhibit from about 10-fold to about 100-fold greater induction of degradation of a BCL-X_{L} protein relative to induction of degradation of an off-target protein. In some embodiments, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, can exhibit from about 100-fold to about 1000-fold greater induction of degradation of a BCL-X_{L} protein relative to induction of degradation of an off-target protein. In some embodiments, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, can exhibit from about 1000-fold to about 10000-fold greater induction of degradation of a BCL-X_{L} protein relative to induction of degradation of an off-target protein.

Certain agents that inhibit or induce degradation of BCL-X_{L} have demonstrated platelet toxicity, which resulted in dose-limiting toxicity (e.g., thrombocytopenia) in the clinic. See, e.g., Adams and Cory, Cell Death & Differentiation 25.1 (2018): 27-36; Campbell and Tait. Open Biology 8.5 (2018): 180002; Pullarkat et al., Cancer Discovery (2021) 10.1158/2159-8290.CD-20-1465; Negi and Voisin-Chiret. ChemBioChem (2022) (doi: 10.1002/cbic.202100689). The viability of platelets may be monitored with any appropriate assay, such as those described herein. See, for instance, Example B6.

In some embodiments, a therapeutically effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, shows at least about 30% platelet viability (e.g., at least about 50% platelet viability, or at least about 80% platelet viability) when administered to a subject. In some embodiments, a therapeutically effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, shows about 30% to about 100% platelet viability (e.g., about 50% to about 100% platelet viability, or about 80% to about 100% platelet viability) when administered to a subject.

In some embodiments, a therapeutically effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, shows at least about 30% platelet viability (e.g., at least about 50% platelet viability, or at least about 80% platelet viability) when administered to a subject and has a Yₘᵢₙ value of about 50% to about 70%. In some embodiments, a therapeutically effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, shows at least about 30% platelet viability (e.g., at least about 50% platelet viability, or at least about 80% platelet viability) when administered to a subject and has a Yₘᵢₙ value of less than about 50%. In some embodiments, a therapeutically effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, shows at least about 30% platelet viability (e.g., at least about 50% platelet viability, or at least about 80% platelet viability) when administered to a subject and has a Yₘᵢₙ value of about 0% to about 50%.

In some embodiments, a therapeutically effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, shows between about 30% and 100% platelet viability (e.g., about 50% to about 100% platelet viability, or about 80% to about 100% platelet viability) when administered to a subject and has a Yₘᵢₙ value of about 50% to about 70%. In some embodiments, a therapeutically effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, shows between about 30% and 100% platelet viability (e.g., about 50% to about 100% platelet viability, or about 80% to about 100% platelet viability) when administered to a subject and has a Yₘᵢₙ value of less than about 50%. In some embodiments, a therapeutically effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, shows between about 30% and 100% platelet viability (e.g., about 50% to about 100% platelet viability, or about 80% to about 100% platelet viability) when administered to a subject and has a Yₘᵢₙ value of about 0% to about 50%.

In some embodiments, therapeutically effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, shows at least about 50% platelet viability (e.g., at least about 80% platelet viability) when administered to a subject and has a Yₘᵢₙ value of about 50% to about 70%. In some embodiments, a therapeutically effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, shows at least about 50% platelet viability (e.g., at least about 80% platelet viability) when administered to a subject and has a Yₘᵢₙ value of less than about 50%. In some embodiments, a therapeutically effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, shows at least about 50% platelet viability (e.g., at least about 80% platelet viability) when administered to a subject and has a Yₘᵢₙ value of about 0% to about 50%.

In some embodiments, a therapeutically effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, shows between about 50% and 100% platelet viability (e.g., about 80% to about 100% platelet viability) when administered to a subject and has a Yₘᵢₙ value of about 50% to about 70%. In some embodiments, a therapeutically effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, shows between about 50% and 100% platelet viability (e.g., about 80% to about 100% platelet viability) when administered to a subject and has a Yₘᵢₙ value of less than about 50%. In some embodiments, a therapeutically effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, shows between about 50% and 100% platelet viability (e.g., about 80% to about 100% platelet viability) when administered to a subject and has a Yₘᵢₙ value of about 0% to about 50%.

In some embodiments, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, when tested at a concentration of about 0.25 μM to about 3 μM in the assay in Example B6, shows at least about 30% platelet viability (e.g., at least about 50% platelet viability, or at least about 80% platelet viability). In some embodiments, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, when tested at a concentration of about 0.25 μM to about 3 μM in the assay in Example B6, shows about 30% to about 100% platelet viability (e.g., about 50% to about 100% platelet viability, or about 80% to about 100% platelet viability).

In some embodiments, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, when tested at a concentration of about 0.25 μM to about 3 μM in the assay in Example B6, shows at least about 30% platelet viability (e.g., at least about 50% platelet viability, or at least about 80% platelet viability) and has a Yₘᵢₙ value of about 50% to about 70% in the assay described in Example B1. In some embodiments, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (TI) (e.g., (II-a)), or a pharmaceutically acceptable salt thereof, when tested at a concentration of about 0.25 μM to about 3 μM in the assay in Example B6, shows at least about 30% platelet viability (e.g., at least about 50% platelet viability, or at least about 80% platelet viability) and has a Yₘᵢₙ value of less than about 50% in the assay described in Example B1. In some embodiments, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, when tested at a concentration of about 0.25 μM to about 3 μM in the assay in Example B6, shows at least about 30% platelet viability (e.g., at least about 50% platelet viability, or at least about 80% platelet viability) and has a Yₘᵢₙ value of about 0% to about 50% in the assay described in Example B1.

In some embodiments, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, when tested at a concentration of about 0.25 μM to about 3 μM in the assay in Example B6, shows between about 30% and 100% platelet viability (e.g., about 50% to about 100% platelet viability, or about 80% to about 100% platelet viability) and has a Yₘᵢₙ value of about 50% to about 70% in the assay described in Example B1. In some embodiments, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, when tested at a concentration of about 0.25 μM to about 3 μM in the assay in Example B6, shows between about 30% and 100% platelet viability (e.g., about 50% to about 100% platelet viability, or about 80% to about 100% platelet viability) and has a Yₘᵢₙ value of less than about 50% in the assay described in Example B1. In some embodiments, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, when tested at a concentration of about 0.25 μM to about 3 μM in the assay in Example B6, shows between about 30% and 100% platelet viability (e.g., about 50% to about 100% platelet viability, or about 80% to about 100% platelet viability) and has a Yₘᵢₙ value of about 0% to about 50% in the assay described in Example B1.

In some embodiments, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, when tested at a concentration of about 0.25 μM to about 3 μM in the assay in Example B6, shows at least about 50% platelet viability (e.g., at least about 80% platelet viability) and has a Yₘᵢₙ value of about 50% to about 70% in the assay described in Example B1. In some embodiments, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, when tested at a concentration of about 0.25 μM to about 3 μM in the assay in Example B6, shows at least about 50% platelet viability (e.g., at least about 80% platelet viability) and has a Yₘᵢₙ value of less than about 50% in the assay described in Example B1. In some embodiments, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, when tested at a concentration of about 0.25 μM to about 3 μM in the assay in Example B6, shows at least about 50% platelet viability (e.g., at least about 80% platelet viability) and has a Yₘᵢₙ value of about 0% to about 50% in the assay described in Example B1.

In some embodiments, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, when tested at a concentration of about 0.25 μM to about 3 μM in the assay in Example B6, shows between about 50% and 100% platelet viability (e.g., about 80% to about 100% platelet viability) and has a Yₘᵢₙ value of about 50% to about 70% in the assay described in Example B1. In some embodiments, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, when tested at a concentration of about 0.25 μM to about 3 μM in the assay in Example B6, shows between about 50% and 100% platelet viability (e.g., about 80% to about 100% platelet viability) and has a Yₘᵢₙ value of less than about 50% in the assay described in Example B1. In some embodiments, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, when tested at a concentration of about 0.25 μM to about 3 μM in the assay in Example B6, shows between about 50% and 100% platelet viability (e.g., about 80% to about 100% platelet viability) and has a Yₘᵢₙ value of about 0% to about 50% in the assay described in Example B1.

Provided herein is a method of treating a cancer in a subject in need of such treatment, the method comprising administering to the subject a therapeutically effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, or a pharmaceutical composition thereof. In some embodiments, the subject is treatment naïve with respect to the cancer. In some embodiments, the subject has received one or more lines of previous therapy for the cancer.

Also provided herein is a method of treating a cancer in a subject in need thereof, the method comprising administering to the subject a therapeutically effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, or a pharmaceutical composition thereof, as a monotherapy. In some embodiments, the subject is treatment naïve with respect to the cancer. In some embodiments, the subject has received one or more lines of previous therapy for the cancer.

Provided herein is a method of treating a cancer in a subject in need of such treatment, the method comprising:
(a) detecting a biomarker (e.g., a mutation, an amplification, a copy number increase, and/or expression (optionally including level of expression) of the biomarker) associated with the cancer; and
(b) administering to the subject a therapeutically effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, or a pharmaceutical composition thereof, as a monotherapy or in combination with an additional therapy or therapeutic agent.

Also provided herein is a method of treating a cancer in a subject, wherein the subject has been determined (e.g., prior to administration of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a))) to have a biomarker (e.g., a mutation, an amplification, a copy number increase, and/or expression (optionally including level of expression) of the biomarker) associated with the cancer, the method comprising administering to the subject a therapeutically effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, or a pharmaceutical composition thereof, as a monotherapy or in combination with an additional therapy or therapeutic agent.

Provided herein is a method of treating a cancer in a subject in need of such treatment, the method comprising:
(a) detecting a biomarker (e.g., a mutation, an amplification, a copy number increase, and/or expression (optionally including level of expression) of a marker of susceptibility to particular agents (e.g., HER2 expression, ER expression, PR expression, folate receptor expression)) associated with the cancer; and
(b) administering to the subject a therapeutically effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, or a pharmaceutical composition thereof, as a monotherapy or in combination with an additional therapy or therapeutic agent.

Also provided herein is a method of treating a cancer in a subject, wherein the subject has been determined (e.g., prior to administration of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a))) to have a biomarker (e.g., a mutation, an amplification, a copy number increase, and/or expression (optionally including level of expression) of a marker of susceptibility to particular agents (e.g., HER2 expression, ER expression, PR expression, folate receptor expression)) associated with the cancer, the method comprising administering to the subject a therapeutically effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, or a pharmaceutical composition thereof, as a monotherapy or in combination with an additional therapy or therapeutic agent.

Provided herein is use of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, or a pharmaceutical composition thereof, for the treatment of cancer, for example, any of the cancers provided herein.

Provided herein is use of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, or a pharmaceutical composition thereof, as a medicament for the treatment of cancer, for example, any of the cancers provided herein.

Provided herein is use of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, in the manufacture of a medicament for the treatment of cancer, for example, any of the cancers provided herein.

Provided herein is a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, or a pharmaceutical composition thereof, for use as a medicament. Also provided herein is a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, or a pharmaceutical composition thereof, for use as a medicament for the treatment of cancer, for example, any of the cancers provided herein.

Provided herein is a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, or a pharmaceutical composition thereof, for use in treating a cancer, for example, any of the cancers provided herein.

As used herein, “monotherapy”, when referring to a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, means that the compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, is the only therapeutic agent or therapy (e.g., anticancer agent or therapy) administered to the subject during the treatment cycle (e.g., no additional targeted therapeutics, anticancer agents, chemotherapeutics, or checkpoint inhibitors are administered to the subject during the treatment cycle). As a person of ordinary skill in the art would understand, monotherapy does not exclude the co-administration of medicaments for the treatment of side effects or general symptoms associated with the cancer or treatment, such as pain, rash, edema, photosensitivity, pruritis, skin discoloration, hair brittleness, hair loss, brittle nails, cracked nails, discolored nails, swollen cuticles, fatigue, weight loss, general malaise, shortness of breath, infection, anemia, or gastrointestinal symptoms, including nausea, diarrhea, and lack of appetite.

As used herein, “the subject has previously received one or more therapeutic agents or therapies for the cancer” means that the subject has been previously administered one or more therapeutic agents or therapies (e.g., anticancer agent or therapy) for the cancer other than a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, during a prior treatment cycle. In some embodiments, the subject cannot tolerate the one or more therapeutic agents or therapies previously administered for the cancer. In some embodiments, the subject did not respond to the one or more therapeutic agents or therapies previously administered for the cancer. In some embodiments, the subject did not adequately respond to one or more therapeutic agents or therapies previously administered for the cancer. In some embodiments, the subject has stopped responding to the one or more therapeutic agents or therapies previously administered for the cancer. In some embodiments, a lack of response, an inadequate response, or a discontinued response can be determined by objective criteria (e.g., tumor volume, or by criteria such as RECIST 1.1). In some embodiments, a lack of response, an inadequate response, or a discontinued response can be determined by the subject's physician.

As used herein, “the subject is treatment naïve with respect to the cancer” means that the subject has not been previously administered one or more therapeutic agents or therapies for the cancer.

For any of the solid tumors described herein, the solid tumor can be primary tumors or metastatic (or secondary) tumors. As used herein, “primary” tumors are those located at the site where the tumor began to grow (i.e., where it originated). As used herein, “metastatic” (or “secondary”) tumors are those that have spread to other parts of body from the original tumor site. In some embodiments, the metastatic or secondary tumors are the same type of cancer as the primary tumor. In some embodiments, the metastatic or secondary tumors are not genetically identical to the primary tumor.

In some embodiments of any of the methods or uses described herein, the cancer is breast cancer (e.g., breast invasive carcinoma, breast invasive ductal carcinoma), central or peripheral nervous system tissue cancer (e.g., brain cancer (e.g., astrocytoma, glioblastoma, glioma, oligoastrocytoma)), endocrine or neuroendocrine cancer (e.g., adrenal cancer (e.g., adrenocortical carcinoma, neuroblastoma, pheochromocytoma, paraganglioma), multiple neuroendocrine type I and type II tumors, parathyroid cancer, pituitary tumors, thyroid cancer (e.g., papillary thyroid cancer)), eye cancer (e.g., uveal cancer (e.g., uveal melanoma)), gastrointestinal cancer (e.g., anal cancer, bile duct cancer (e.g., cholangiocarcinoma (e.g., intrahepatic cholangiocarcinoma)), colorectal cancer (e.g., colon adenocarcinoma, rectal adenocarcinoma, mucinous adenocarcinoma, mucinous carcinoma), esophageal cancer (e.g., esophageal adenocarcinoma), gallbladder cancer, gastrointestinal carcinoid tumor, gastrointestinal stromal tumor (GIST), liver cancer (e.g., hepatocellular carcinoma, intrahepatic bile duct cancer), pancreatic cancer (e.g., pancreatic adenocarcinoma, pancreatic islet cell cancer), small intestine cancer, or stomach cancer (e.g., stomach adenocarcinoma, signet ring cell carcinoma of the stomach)), genitourinary cancer (e.g., bladder cancer (e.g., bladder urothelial carcinoma), kidney cancer (e.g., renal clear cell carcinoma, renal papillary cell carcinoma, kidney chromophobe), prostate cancer (e.g., prostate adenocarcinoma), testicular cancer (e.g., testicular germ cell tumors), or ureter cancer), gynecologic cancer (e.g., cervical cancer (e.g., cervical squamous cell carcinoma, endocervical adenocarcinoma, mucinous carcinoma), ovarian cancer (e.g., serous ovarian cancer, ovarian serous cystadenocarcinoma), uterine cancer (e.g., uterine carcinosarcoma, uterine endometrioid carcinoma, uterine serous carcinoma, uterine papillary serous carcinoma, uterine corpus endometrial carcinoma), or vulvar cancer), head and neck cancer (e.g., ear cancer (e.g., middle ear cancer), head and neck squamous cell carcinoma, nasal cavity cancer, oral cancer, pharynx cancer (e.g., hypopharynx cancer, nasopharynx cancer, oropharyngeal cancer), hematological cancer (e.g., leukemia (e.g., chronic eosinophilic leukemia (CEL), chronic lymphocytic leukemia (CLL), chronic neutrophilic leukemia (CNL), acute lymphocytic leukemia (ALL) (e.g., Philadelphia chromosome positive ALL or T cell ALL (T-ALL)), acute myeloid leukemia (AML) (e.g., acute promyelocytic leukemia (APL), post-MPN AML, post-myelodysplastic syndrome (post-MDS) AML, M6-AML (also known as pure erythroid leukemia (PEL)), or M7-AML (also known as acute megakaryoblastic leukemia (AKML))), chronic myeloid leukemia (CML)), lymphoma (e.g., Hodgkin lymphoma (e.g., nodular lymphocyte predominant Hodgkin lymphoma (NLPHL)), or non-Hodgkin lymphoma (e.g., Burkitt lymphoma (BL), diffuse large B cell lymphoma (DLBCL), diffuse histiocytic lymphoma (DHL), follicular lymphoma (FL), intravascular large B cell lymphoma (IVLBCL), mantle cell lymphoma (MCL), small lymphocytic lymphoma (SLL), a T cell lymphoma (e.g., anaplastic large T cell lymphoma, cutaneous T cell lymphoma, or peripheral T cell lymphoma))), essential thrombocythemia, polycythemia vera, myelofibrosis (e.g., primary myelofibrosis, post-essential thrombocythemia myelofibrosis, or post-polycythemia vera myelofibrosis), a myelodysplastic syndrome (MDS) (e.g., M6 MDS or M7 MDS), or myeloma (e.g., multiple myeloma)), Li-Fraumeni tumors, mesentery cancer (e.g., omentum cancer, peritoneal cancer), pleural cancer, respiratory cancer (e.g., larynx cancer, lung cancer (e.g., lung squamous cell carcinoma, lung adenocarcinoma, malignant pleural mesothelioma, non-small cell lung cancer (NSCLC), small cell lung cancer), tracheal cancer), sarcoma (e.g., bone cancer (e.g., osteosarcoma, chondrosarcoma) or soft tissue sarcoma (Ewing sarcoma, leiomyosarcoma, myxofibrosarcoma, rhabdomyosarcoma)), skin cancer (e.g., melanoma or Merkel cell carcinoma), thymus cancer (e.g., thymoma), or a combination thereof.

In some embodiments, the cancer is breast cancer, gastrointestinal cancer (e.g., bile duct cancer (e.g., cholangiocarcinoma (e.g., intrahepatic cholangiocarcinoma)), colorectal cancer (CRC), gastrointestinal stromal tumor, or pancreatic cancer), genitourinary cancer (e.g., bladder cancer (e.g., bladder urothelial carcinoma) or kidney cancer), gynecologic cancer (e.g., cervical cancer, ovarian cancer (e.g., high grade serous ovarian cancer (HGSOC), low grade serous ovarian cancer (LGSOC)), or uterine cancer), head and neck cancer (e.g., head and neck squamous cell carcinoma), hematological cancer (e.g., leukemia (e.g., acute lymphocytic leukemia (ALL) (e.g., T-ALL), acute myeloid leukemia (AML) (e.g., APL, post-MPN AML, post-MDS AML, M6-AML, or M7-AML), chronic lymphocytic leukemia (CLL)), lymphoma (e.g., follicular lymphoma (FL), small lymphocytic lymphoma (SLL), a T cell lymphoma (e.g., anaplastic large T cell lymphoma, cutaneous T cell lymphoma, or peripheral T cell lymphoma), or diffuse large B cell lymphoma (DLBCL)), essential thrombocythemia, polycythemia vera, myelofibrosis (e.g., primary myelofibrosis, post-essential thrombocythemia myelofibrosis, or post-polycythemia vera myelofibrosis), or a myelodysplastic syndrome (MDS) (e.g., M6 MDS or M7 MDS)), lung cancer (e.g., non small cell lung cancer (NSCLC), small cell lung cancer (SCLC), lung squamous cell carcinoma, or malignant pleural mesothelioma (MPM)), neuroblastoma, sarcoma (e.g., bone cancer (e.g., osteosarcoma, chondrosarcoma) or soft tissue sarcoma (Ewing sarcoma, leiomyosarcoma, myxofibrosarcoma, rhabdomyosarcoma)), or skin cancer (e.g., melanoma or Merkel cell carcinoma).

In some embodiments, the cancer is a myeloproliferative neoplasm (MPN). In some embodiments, the myeloproliferative neoplasm is CEL, CML, CNL, essential thrombocythemia, polycythemia vera, or myelofibrosis (e.g., primary myelofibrosis, post-essential thrombocythemia myelofibrosis, or post-polycythemia vera myelofibrosis). In some embodiments, the MPN has a JAK2 mutation (e.g., a JAK2 V617F mutation). In some embodiments, the MPN does not have a JAK2 mutation. In some embodiments, the cancer is low-risk myelofibrosis. In some embodiments, the cancer is intermediate (e.g., intermediate-1 and/or intermediate-2) or high-risk myelofibrosis (e.g., primary myelofibrosis, post-essential thrombocythemia myelofibrosis, or post-polycythemia vera myelofibrosis). In some embodiments, the cancer is intermediate or high-risk myelofibrosis (e.g., primary myelofibrosis, post-essential thrombocythemia myelofibrosis, or post-polycythemia vera myelofibrosis) with a JAK2 mutation (e.g., a JAK2 V617F mutation). In some embodiments, the cancer is intermediate or high-risk myelofibrosis (e.g., primary myelofibrosis, post-essential thrombocythemia myelofibrosis, or post-polycythemia vera myelofibrosis) without a JAK2 V617F mutation. In some embodiments, the subject has a platelet count below 50×10⁹/L.

In some embodiments, the cancer is polycythemia vera. In some embodiments, the cancer is polycythemia vera, and the subject has had an inadequate response to or is intolerant of hydroxyurea.

In some embodiments, the cancer is an MDS. In some embodiments, the cancer is M6 MDS. In some embodiments, the cancer is M7 MDS.

In some embodiments, the cancer is T-ALL. In some embodiments, the cancer is relapsed/refractory T-ALL.

In some embodiments, the cancer is CRC (e.g., Braf mutant CRC (e.g., Braf V600E CRC) or KRas mutant CRC (e.g., KRas G12C CRC or KRas G12D CRC)).

In some embodiments, the cancer is SCLC (e.g., ASCL1 subtype SCLC or NEUROD1 subtype SCLC).

In some embodiments, the cancer is NSCLC (e.g., Braf mutant NSCLC (e.g., Braf V600E NSCLC), EGFR mutant NSCLC (e.g., EGFR L858R NSCLC or EGFR exon 19 deletion NSCLC), MET mutant NSCLC (e.g., MET exon 14 deletion NSCLC, MET amplified NSCLC), KRas mutant NSCLC (e.g., KRas G12C NSCLC)).

In some embodiments, the cancer is lung squamous cell carcinoma.

In some embodiments, the cancer is malignant pleural mesothelioma (e.g., BAP1 mutant malignant pleural mesothelioma). In some embodiments, the cancer is malignant pleural mesothelioma, and a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, is administered as a monotherapy.

In some embodiments, the cancer is melanoma (e.g., Braf mutant melanoma (e.g., Braf V600E melanoma)).

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), HER2 low breast cancer, triple negative breast cancer, hormone receptor positive breast cancer (ER+ and/or PR+, with or without HER2 positivity)).

In some embodiments, the cancer is lymphoma. In some embodiments, the lymphoma is a T cell lymphoma (e.g., anaplastic large T cell lymphoma, cutaneous T cell lymphoma, or peripheral T cell lymphoma). In some embodiments, the lymphoma is a non-Hodgkin lymphoma (e.g., DLBCL, anaplastic large T cell lymphoma, cutaneous T cell lymphoma, or peripheral T cell lymphoma). In some embodiments, the lymphoma is peripheral T cell lymphoma.

In some embodiments, the cancer is leukemia. In some embodiments, the leukemia is a T cell leukemia (e.g., T cell ALL). In some embodiments, the cancer is post-MPN leukemia. In some embodiments, the cancer is a T cell leukemia (e.g., T cell ALL), and a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, is administered as a monotherapy.

In some embodiments, the cancer is M6-AML. In some embodiments, the M6-AML is a post-MPN AML. In some embodiments, the M6-AML is a post-myelodysplastic syndrome (MDS) AML. In some embodiments, the cancer is M6-AML, and the compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or pharmaceutically acceptable salt thereof, is administered as a monotherapy.

In some embodiments, the cancer is M7-AML. In some embodiments, the M7-AML is a post-MPN AML. In some embodiments, the M7-AML is a post-MDS AML. In some embodiments, the cancer is M7-AML, and the compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or pharmaceutically acceptable salt thereof, is administered as a monotherapy.

In some embodiments, the cancer is head and neck cancer.

In some embodiments, the cancer is essential thrombocythemia.

In some embodiments, the cancer is polycythemia vera.

In some embodiments, the cancer is myelofibrosis. In some embodiments, the cancer is primary myelofibrosis. In some embodiments, the cancer is post-essential thrombocythemia myelofibrosis. In some embodiments, the cancer is post-polycythemia vera myelofibrosis.

In some embodiments, the cancer is an MDS. In some embodiments, the MDS is M6 MDS. IN some embodiments, the MDS is M7 MDS. In some embodiments, the cancer is an MDS, and a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, is administered as a monotherapy.

In some embodiments, the cancer is pancreatic cancer.

In some embodiments, the cancer is bladder cancer (e.g., bladder urothelial carcinoma).

In some embodiments, the cancer is ovarian cancer (e.g., BRCA1 mutant ovarian cancer or BRCA2 mutant ovarian cancer). In some embodiments, the cancer is HGSOC (e.g., BRCA1 mutant HGSOC or BRCA2 mutant HGSOC).

In some embodiments, the cancer is cervical cancer.

In some embodiments, the cancer is colorectal cancer.

In some embodiments, the cancer is skin cancer. In some embodiments, the skin cancer is melanoma. In some embodiments, the cancer is Merkel cell carcinoma. In some embodiments, the cancer is Merkel cell carcinoma, and a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, is administered as a monotherapy.

In some embodiments, the cancer is neuroblastoma.

In some embodiments, the cancer is intrahepatic cholangiocarcinoma.

In some embodiments, the cancer is a mesenchymal cancer. In some embodiments, the mesenchymal cancer is mesenchymal breast cancer or mesenchymal kidney cancer.

In some embodiments, a BCL-X_{L} copy number gain or a BCL-X_{L} amplification can be detected in a sample from the subject (e.g., detecting three or more copies of a BCL2L1 gene in the sample from the subject). In some embodiments, the subject was determined (e.g., prior to administration of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a))) to have a cancer having a BCL-X_{L} copy number gain or a BCL-X_{L} amplification.

In some embodiments, the cancer has a BCL-X_{L} copy number gain.

In some embodiments, the cancer has a BCL-X_{L} amplification.

Non-limiting examples of BCL-X_{L} involvement in cancers can be found in: Wilson, Wyndham H., et al. The Lancet Oncology 11.12 (2010): 1149-1159; Keitel, Ulrike, et al. Oncotarget 5.23 (2014): 11778; Chonghaile, Triona Ni, et al. Cancer Discovery 4.9 (2014): 1074-1087; Zaanan, Aziz, et al. Journal of Biological Chemistry 290.39 (2015): 23838-23849; Zhang, Haichao, et al. Molecular Cancer 14.1 (2015): 1-9; Soderquist, Ryan S., et al. Nature Communications 9.1 (2018): 1-13; Stover, Elizabeth H., et al. Molecular Cancer Research 17.11 (2019): 2281-2293; Concoran, R. B., et al. Annals of Oncology (2019) 30 (suppl_5): v159-v193; Lakhani, Nehal J., et al. Journal of Clinical Oncology (2020): 3509-3509 doi: 10.1200/JCO.2020.38; He, Yonghan, et al. Journal ofHematology & Oncology 13.1 (2020): 1-13; Grubb, Treg, et al. Clinical Cancer Research (2022) doi: 10.1158/1078-0432.CCR-22-0669; Joly, Florence, et al. Gynecologic Oncology 165.1 (2022): 30-39; and Nanjo, Shigeki, et al. The Journal of ClinicalInvestigation (2022) doi: 10.1172/JCI145099.

In some embodiments, the subject has previously been treated with another anticancer agent, a chemotherapeutic agent, radiation, surgery, a multi-kinase inhibitor, or a combination thereof.

Provided herein is a method of treating an ocular disease or condition in a subject in need of such treatment, the method comprising administering (e.g., intravitreally or topically) to the subject a therapeutically effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, or a pharmaceutical composition thereof. In some embodiments, the ocular disease or condition is diabetic macular edema. In some embodiments, the ocular disease or condition is age-related macular degeneration. In some embodiments, the ocular disease or condition is diabetic retinopathy. See, e.g., Crespo-Garcia, Sergio, et al. Cell Metabolism, 33.4 (2021): 818-832; Hassan, Jannah Waled, and Ashay D. Bhatwadekar, Frontiers in Pharmacology 13 (2022): 896907.

Provided herein is use of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, or a pharmaceutical composition thereof, for the treatment of an ocular disease or condition, for example, any of the ocular diseases or conditions provided herein.

Provided herein is use of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, or a pharmaceutical composition thereof, as a medicament for the treatment of an ocular disease or condition, for example, any of the ocular diseases or conditions provided herein.

Provided herein is use of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, in the manufacture of a medicament for the treatment of an ocular disease or condition, for example, any of the ocular diseases or conditions provided herein.

Also provided herein is a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, or a pharmaceutical composition thereof, for use as a medicament for the treatment of an ocular disease or condition, for example, any of the ocular diseases or conditions provided herein.

Provided herein is a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, or a pharmaceutical composition thereof, for use in treating an ocular disease or condition, for example, any of the ocular diseases or conditions provided herein.

Also provided herein is a method of treating a fibrotic disease or condition and/or a disease or condition associated with senescent cells in a subject in need of such treatment, the method comprising administering to the subject a therapeutically effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, or a pharmaceutical composition thereof. Non-limiting examples of fibrotic diseases or conditions and/or diseases or conditions associated with senescent cells include pulmonary fibrosis (e.g., idiopathic pulmonary fibrosis, systemic fibrosis-associated lung disease, radiation-associated pulmonary fibrosis), radiation-associated skin fibrosis, liver fibrosis, primary sclerosing cholangitis, diabetic macular edema, age-related macular degeneration, diabetic retinopathy, geographic atrophy, ischemia and reperfusion injury, heart failure, recovery from acute myocardial infarction, pulmonary hypertension, inflammatory bowel disease, colitis, Crohn's disease, diabetes, aging skin (including photoaging-related pigmentation), donor organ transplant survival and function, stem cell transplant survival and function, osteoarthritis, recovery from spinal cord injury, Alzheimer's disease, tau-opathies, progressive supranuclear palsy, and age-related neurological decline (e.g., related to impaired neurovascular coupling). See, e.g., Zhu, Y. L, et al. Aging Cell 14.4 (2015): 644-658; Zhu, Y. L, et al. Aging Cell 15.3 (2016): 428-435; Chang, Jianhui, et al. Nature Medicine 22.1 (2016): 78-83; Zhu, Yi, et al. Aging (Albany NY) 9.3 (2017): 955; Lagares, David, et al. Science Translational Medicine 9.420 (2017): eaa13765; Pan, Jin, et al. International Journal of Radiation Oncology Biolog Physics 99.2 (2017): 353-361; Bussian, Tyler J., et al. Nature 562.7728 (2018): 578-582; Moncsek, Anja, et al. Hepatology 67.1 (2018): 247-259; van Willigenburg, Hester, Peter L J de Keizer, and Ron W F de Bruin. Pharmacological Research 130 (2018): 322-330; Walaszczyk, Anna, et al. Aging Cell 18.3 (2019): e12945; Aguayo-Mazzucato, Cristina, et al. Cell Metabolism 30.1 (2019): 129-142; Sessions, Garrett A., et al. The FASEB Journal 33.11 (2019): 12364; Gerdes, Erin O. Wissler, et al. International Review of Neurobiology 155 (2020): 203-234; Sasaki, Motoko, Yasunori Sato, and Yasuni Nakanuma. Journal of Autoimmunity 107 (2020): 102377; Yabluchanskiy, Andriy, et al. Geroscience 42 (2020): 409-428; Dookun, Emily, et al. Aging Cell 19.10 (2020): e13249; Jia, Kangni, et al. Journal of Cardiovascular Pharmacology 76.4 (2020): 452-460; Sierra-Ramirez, Arantzazu, et al. Aging (Albany NY) 12.12 (2020): 11337; Yang, Hao, et al. Aging (Albany NY) 12.13 (2020): 12750: Lawrie, Allan, and Sheila E. Francis. The Journal of Clinical Investigation 131.11 (2021): e14972.1; Paramos-de-Carvalho, Diogo, et al. Cell Reports 36.1 (2021): 109334; Tarantini, Stefano, et al. GeroScience 43.5 (2021): 2427-2440; Park, Ji Hee, et al. The British Journal of Dermatology (2021); Fielder, Edward, et al. ELife 11 (2022): e75492; Suzuki, Keiji, et al. Mutation Research Genetic Toxicology and Environmental Mutagenesis 876 (2022): 503448; He, An, et al. American Journal of Transplantation 22.11 (2022): 2529-2547; Johnson, Laura A., et al. Inflammatory Bowel Diseases 28.2 (2022): 161-175; Miura, Yugo, et al. Stem Cell Research & Therapy 13.1 (2022): 222; Cooley, Joseph C., et al. JCI Insight 8.3 (2023): e163762. Watanabe, Yusuke, et al. Hepatology Research 53 (2023): 460-472; and Takaya, Kento, et al., Rejuvenation Research 26.1 (2023): 9-20.

Provided herein is use of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, or a pharmaceutical composition thereof, for the treatment of a fibrotic disease or condition and/or a disease or condition associated with senescent cells, for example, any of the fibrotic diseases or conditions and/or diseases or conditions associated with senescent cells provided herein.

Provided herein is use of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, or a pharmaceutical composition thereof, as a medicament for the treatment of a fibrotic disease or condition and/or a disease or condition associated with senescent cells, for example, any of the fibrotic diseases or conditions and/or diseases or conditions associated with senescent cells provided herein.

Provided herein is use of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (I-a)), or a pharmaceutically acceptable salt thereof, in the manufacture of a medicament for the treatment of a fibrotic disease or condition and/or a disease or condition associated with senescent cells, for example, any of the fibrotic diseases or conditions and/or diseases or conditions associated with senescent cells provided herein.

Also provided herein is a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, or a pharmaceutical composition thereof, for use as a medicament for the treatment of a fibrotic disease or condition and/or a disease or condition associated with senescent cells, for example, any of the fibrotic diseases or conditions and/or diseases or conditions associated with senescent cells provided herein.

Provided herein is a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, or a pharmaceutical composition thereof, for use in treating a fibrotic disease or condition and/or a disease or condition associated with senescent cells, for example, any of the fibrotic diseases or conditions and/or diseases or conditions associated with senescent cells provided herein.

Also provided is a method for modulating (e.g., decreasing) BCL-X_{L} protein activity in a cell, comprising contacting the cell with an effective compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I—F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, to a subject. In some embodiments, the cell is a cancer cell. In some embodiments, the cell is a mammalian cell. In some embodiments, the cell is a mammalian cancer cell. In some embodiments, the cancer cell is any cancer as described herein.

As used herein, the term “contacting” refers to the bringing together of indicated moieties in an in vitro system or an in vivo system. For example, “contacting” a cell with a compound provided herein includes the administration of a compound provided herein to the cell, in vitro or in vivo, including, for example, introducing a compound provided herein into a sample containing cells (e.g., grown in culture or derived from a patient), an organoid, or an organism (e.g., an animal (e.g., an animal bearing a tumor), or a human).

Also provided is a method of modulating (e.g., decreasing) the level of BCL-X_{L} protein in a cell, comprising contacting the cell with a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof. In some embodiments, the level of BCL-X_{L} protein is decreased by at least 30% (e.g., at least 40%, at least 50%, at least 60%, at least 70%, at least 80%, at least 90%, at least 95%, at least 97%, or at least 99%) compared to a cell not contacted with the compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, to a subject having a cell having a BCL-X_{L} protein. In some embodiments, the cell is a cancer cell. In some embodiments, the cell is a mammalian cell. In some embodiments, the cell is a mammalian cancer cell. In some embodiments, the cancer cell is any cancer as described herein.

Also provided is a method of inducing ubiquitination of a BCL-X_{L} protein in a cell, comprising contacting the cell with a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, to a subject having a cell having a BCL-X_{L} protein. In some embodiments, the cell is a cancer cell. In some embodiments, the cell is a mammalian cell. In some embodiments, the cell is a mammalian cancer cell. In some embodiments, the cancer cell is any cancer as described herein.

Also provided is a method of forming a ternary complex comprising a BCL-X_{L} protein, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, and a CRBN protein or fragment thereof in a cell, comprising contacting the cell with a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, to a subject having a cell having a BCL-X_{L} protein. In some embodiments, the cell is a cancer cell. In some embodiments, the cell is a mammalian cell. In some embodiments, the cell is a mammalian cancer cell. In some embodiments, the cancer cell is any cancer as described herein.

Also provided herein is a method of inhibiting cell proliferation, in vitro or in vivo, the method comprising contacting a cell with an effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, or a pharmaceutical composition thereof as defined herein.

Further provided herein is a method of increasing cell death, in vitro or in vivo, the method comprising contacting a cell with an effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, or a pharmaceutical composition thereof as defined herein. Also provided herein is a method of increasing tumor cell death in a subject. The method comprises administering to the subject a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, in an amount effective to increase tumor cell death.

When employed as pharmaceuticals, the compounds of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or pharmaceutically acceptable salts thereof, can be administered in the form of pharmaceutical compositions as described herein.

Also provided herein is a method for inducing degradation of a BCL-X_{L} protein in a mammalian cell, the method comprising contacting the mammalian cell with an effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof.

Also provided herein is a method of treating a subject having a cancer, wherein the method comprises:
administering a therapeutically effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, as a monotherapy or in conjunction with a first anticancer agent to the subject who has been administered one or more doses of the first anticancer agent to the subject for a period of time.

Also provided herein is a method of treating a subject having a cancer, wherein the method comprises:
(a) administering one or more doses of a first anticancer agent to the subject for a period of time; and
(b) after (a), administering a therapeutically effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, as a monotherapy or in conjunction with the first anticancer agent to the subject.

Also provided herein is a method of treating a subject having a cancer, wherein the method comprises:
(a) administering one or more doses of a first anticancer agent to the subject for a period of time; and
(b) after (a), administering a therapeutically effective amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, as a monotherapy or in conjunction with a second anticancer agent to the subject.

Combinations

In any of the indications described herein, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, can be used as a monotherapy. In some embodiments, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, can be used prior to administration of an additional therapeutic agent or additional therapy. For example, a subject in need thereof can be administered one or more doses of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, for a period of time and then undergo at least partial resection of the tumor. In some embodiments, the treatment with one or more doses of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, reduces the size of the tumor (e.g., the tumor burden) prior to the at least partial resection of the tumor.

In some embodiments, a subject in need thereof can be administered one or more doses of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, for a period of time and under one or more rounds of radiation therapy. In some embodiments, the treatment with one or more doses of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, reduces the size of the tumor (e.g., the tumor burden) prior to the one or more rounds of radiation therapy.

In some embodiments of any the methods described herein, the compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, is administered in combination with a therapeutically effective amount of at least one additional therapeutic agent selected from one or more additional therapies or therapeutic (e.g., chemotherapeutic) agents.

Non-limiting examples of additional therapies and therapeutic agents include: RAS pathway targeted therapeutic agents (e.g., Ras/RAF/MEK/PI3K pathway inhibitors, (e.g., Ras inhibitors (e.g., KRas inhibitors), KRas-targeted therapeutic agents, SOS1 inhibitors, SOS1/Ras protein-protein interaction inhibitors, SHP2 inhibitors, PI3K-AKT-mTOR pathway inhibitors)), kinase-targeted therapeutics (e.g., MEK inhibitors, ERK inhibitors, Raf inhibitors (e.g., BRaf inhibitors), PI3K inhibitors, Abl inhibitors (e.g., BCR-Abl inhibitors), ALK inhibitors, AKT inhibitors, AURKA inhibitors, mTOR inhibitors, CDK2 inhibitors, CDK4/5 inhibitors, CDK4/6 inhibitors, CDK7 inhibitors, CDK9 inhibitors, MET (also known as cMET) inhibitors, FAK inhibitors, FGFR1 inhibitors, FGFR2 inhibitors, FGFR3 inhibitors, FGFR4 inhibitors, ErbB family inhibitors (e.g., EGFR inhibitors, anti-EGFR antibodies or anti-EGFR antibody-drug conjugates, HER2 inhibitors, anti-HER2 antibodies or anti-HER2 antibody-utilizing biologics), JAK inhibitors (e.g., JAK inhibitors having activity against JAK1, JAK2, and/or JAK1/2), Src inhibitors, VEGFR inhibitors), LSD1 inhibitors, EZH2 inhibitors, BET inhibitors, STING agonists, telomerase inhibitors, mTORC1 inhibitors, YAP inhibitors, proteasome inhibitors, farnesyl transferase inhibitors, Hif2α inhibitors, HSP90 inhibitors, PTEN inhibitors, PARP inhibitors, signal transduction pathway inhibitors, checkpoint inhibitors, modulators of the apoptosis pathway (e.g., BCL-2 inhibitors), chemotherapeutics, angiogenesis-targeted therapies, immune-targeted agents, including immunomodulatory imide drugs (sometimes called “IMiDs” or “CELMoDs”) and immunotherapy (e.g., anti-PD1 therapy or anti-PD-L1 therapy), and radiotherapy.

As used herein, a biosimilar antibody refers to an antibody or antigen-binding fragment that has the same primary amino acid sequence as compared to a reference antibody and optionally, may have detectable differences in post-translation modifications (e.g., glycosylation and/or phosphorylation) as compared to the reference antibody (e.g., a different glycoform).

In some embodiments, the additional therapy or therapeutic agent is an Abl inhibitor (e.g., a BCR-Abl inhibitor), an ALK inhibitor, an AURKA inhibitor, a BCL-2 inhibitor, a Braf inhibitor, a CDK2 inhibitor, a CDK4/6 inhibitor, a CDK7 inhibitor, a CDK9 inhibitor, an EGFR inhibitor, an anti-EGFR antibody or anti-EGFR antibody-drug conjugate, an ERK inhibitor, an EZH2 inhibitor, a FGFR1 inhibitor, a FGFR2 inhibitor, a FGFR3 inhibitor, a FGFR4 inhibitor, a HER2 inhibitor, an anti-HER2 antibody or anti-HER2 antibody-drug conjugate, a JAK inhibitor, a KRas inhibitor, a MEK inhibitor, a MET inhibitor, a Hif2α inhibitor, a PARP inhibitor, a VEGFR inhibitor, an LSD1 inhibitor, a BET inhibitor, a STING agonist, a telomerase inhibitor, a TORC1/2 inhibitor, an immunomodulatory imide drug, immunotherapy (e.g., a PD-1 inhibitor (e.g., anti-PD1 therapy), a PD-L1 inhibitor (e.g., anti-PD-L1 therapy)), chemotherapy, radiotherapy, or a combination thereof.

In some embodiments, the additional therapy or therapeutic agent is an Abl degrader (e.g., a BCR-Abl degrader), an ALK degrader, an AURKA degrader, a BCL-2 degrader, a BRaf degrader, a CDK2 degrader, a CDK4/6 degrader, a CDK7 degrader, a CDK9 degrader, an EGFR degrader, an ERK degrader, an EZH2 degrader, a FGFR1 degrader, a FGFR2 degrader, a FGFR3 degrader, a FGFR4 degrader, a HER2 degrader, a JAK2 degrader, a KRas degrader, a MEK degrader, a MET degrader, a Hif2α degrader, a PARP degrader, a VEGFR degrader, an LSD1 degrader, a BET degrader, a telomerase degrader, a TORC1/2 degrader, an immunomodulatory imide drug, immunotherapy (e.g., anti-PD1 therapy or anti-PD-L1 therapy), chemotherapy, radiotherapy, or a combination thereof.

In some embodiments, the Abl inhibitor (e.g., BCR-Abl inhibitor) is asciminib (e.g., asciminib hydrochloride), bafetinib, bosutinib (e.g., bosutinib monohydrate), danusertib, dasatinib (e.g., dasatinib monohydrate), flumatinib (e.g., flumatinib mesylate), imatinib (e.g., imatinib mesylate), nilotinib (e.g., nilotinib monochloride monohydrate), olverembatinib (e.g., olverembatinib mesylate), ponatinib (e.g., ponatinib hydrochloride), radotinib (e.g., radotinib dihydrochloride), ruserontinib, vandetanib, AN-019, AT-9283, IkT-148009, NPB-001-056, or a combination thereof.

In some embodiments, the ALK inhibitor is alectinib (e.g., alectinib hydrochloride), brigatinib, ceritinib, crizotinib, ensartinib (e.g., ensartinib hydrochloride), entrectinib, fidrisertib, lorlatinib, TQ-B-3101, TQ-B-3139, or a combination thereof. In some embodiments, the ALK inhibitor is alectinib (e.g., alectinib hydrochloride), brigatinib, ceritinib, crizotinib, ensartinib (e.g., ensartinib hydrochloride), fidrisertib, lorlatinib, TQ-B-3101, TQ-B-3139, or a combination thereof.

In some embodiments, the AURKA inhibitor is alisertib, danusertib, ilorasertib, tinengotinib, AT-9283, BI-811283, ENMD-2076, or a combination thereof.

In some embodiments, the BCL-2 inhibitor is lisaftoclax, navitoclax, obatoclax, venetoclax, oblimersen (e.g., oblimersen sodium), beclanorsen, AZD-0466, BGB-11417, UBX-1325 (or a phosphate prodrug thereof), UBX-1967 (or a phosphate prodrug thereof), ZN-d5, or a combination thereof.

In some embodiments, the cancer is a lung cancer (e.g., SCLC), and the additional therapy or therapeutic agent is a BCL-2 inhibitor (e.g., lisaftoclax, navitoclax, obatoclax, venetoclax, oblimersen (e.g., oblimersen sodium), beclanorsen, AZD-0466, BGB-11417, UBX-1325 (or a phosphate prodrug thereof), UBX-1967 (or a phosphate prodrug thereof), or ZN-d5). In some embodiments, the cancer is a lung cancer (e.g., SCLC), and the additional therapy or therapeutic agent is venetoclax.

In some embodiments, the cancer is a non-Hodgkin lymphoma, and the additional therapy or therapeutic agent is a BCL-2 inhibitor (e.g., lisaftoclax, navitoclax, obatoclax, venetoclax, oblimersen (e.g., oblimersen sodium), beclanorsen, AZD-0466, BGB-11417, UBX-1325 (or a phosphate prodrug thereof), UBX-1967 (or a phosphate prodrug thereof), or ZN-d5). In some embodiments, the cancer is a non-Hodgkin lymphoma, and the additional therapy or therapeutic agent is venetoclax.

In some embodiments, the BRaf inhibitor is avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, RO5185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, PLX-4720, or a combination thereof.

In some embodiments, a BRaf V600E mutation can be detected in a sample from the subject (e.g., detecting a BRAF gene having a mutation corresponding to a V600E mutation in BRaf protein and/or detecting a BRaf protein having a V600E mutation). In some embodiments, the subject was determined (e.g., prior to administration of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (I-a))) to have a cancer having a BRaf V600E mutation

In some embodiments, the cancer is a BRaf mutant CRC (e.g., BRaf V600E mutant CRC), and the additional therapy or therapeutic agent is a BRaf inhibitor (e.g., avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, RO5185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, or PLX-4720). In some embodiments, the cancer is a BRaf mutant CRC (e.g., BRaf V600E mutant CRC), and the additional therapy or therapeutic agent is a BRaf inhibitor (e.g., avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, RO5185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, or PLX-4720) and an anti-EGFR antibody or anti-EGFR antibody-drug conjugate (e.g., amivantamab (e.g., amivantamab-vmjw, or a biosimilar thereof), cetuximab (e.g., ERBITUX® (cetuximab), or a biosimilar thereof (e.g., CMAB-009, CPGJ-602, or KL-140)), cetuximab sarotalocan (AKALUX® (cetuximab sarotalocan), or a biosimilar thereof), depatuxizumab, duligotuzumab, futuximab, imgatuzumab, modotuximab, necitumumab (e.g., PORTRAZZA® (necitumumab), or a biosimilar thereof), nimotuzumab (e.g., BIOMAb EGFR® (nimotuzumab), or a biosimilar thereof), panitumumab (e.g., VECTIBIX® (panitumumab), or a biosimilar thereof), tomuzotuximab, zalutumumab, EMD-55900, EMD-82633, GC-1118, HLX-07, ICR-62, SCT-200, SI-B-001, TAS-0313, or biosimilars thereof). In some embodiments, the cancer is a BRaf mutant CRC (e.g., BRaf V600E mutant CRC), and the additional therapy or therapeutic agent is a BRaf inhibitor (e.g., avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, RO5185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, or PLX-4720) and cetuximab (e.g., ERBITUX® (cetuximab), or a biosimilar thereof (e.g., CMAB-009, CPGJ-602, or KL-140)). In some embodiments, the cancer is a BRaf mutant CRC (e.g., BRaf V600E mutant CRC), and the additional therapy or therapeutic agent is a BRaf inhibitor (e.g., avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, RO5185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, or PLX-4720) and panitumumab (e.g., VECTIBIX® (panitumumab), or a biosimilar thereof). In some embodiments, the cancer is a BRaf mutant CRC (e.g., BRaf V600E mutant CRC), and the additional therapy or therapeutic agent is a dabrafenib (e.g., dabrafenib mesylate, GSK2118436), encorafenib (e.g., BRAFTOVI™, LGX818), or vemurafenib (e.g., ZELBORAF®, RO5185426) and cetuximab (e.g., ERBITUX® (cetuximab), or a biosimilar thereof (e.g., CMAB-009, CPGJ-602, or KL-140)).

In some embodiments, the cancer is a BRaf mutant CRC (e.g., BRaf V600E mutant CRC), and the additional therapy or therapeutic agent is dabrafenib (e.g., dabrafenib mesylate, GSK2118436), encorafenib (e.g., BRAFTOVI™, LGX818), or vemurafenib (e.g., ZELBORAF®, RO5185426) and panitumumab (e.g., VECTIBIX® (panitumumab), or a biosimilar thereof). In some embodiments, the cancer is a BRaf mutant CRC (e.g., BRaf V600E mutant CRC), and the additional therapy or therapeutic agent is a dabrafenib (e.g., dabrafenib mesylate, GSK2118436) and cetuximab (e.g., ERBITUX® (cetuximab), or a biosimilar thereof (e.g., CMAB-009, CPGJ-602, or KL-140)). In some embodiments, the cancer is a BRaf mutant CRC (e.g., BRaf V600E mutant CRC), and the additional therapy or therapeutic agent is dabrafenib (e.g., dabrafenib mesylate, GSK2118436) and panitumumab (e.g., VECTIBIX® (panitumumab), or a biosimilar thereof).

In some embodiments, the cancer is a BRaf mutant NSCLC (e.g., BRaf V600E mutant NSCLC), and the additional therapy or therapeutic agent is a BRaf inhibitor (e.g., avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, RO5185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, or PLX-4720) and a MEK inhibitor (e.g., avutometinib, binimetinib, cobimetinib (e.g., cobimetinib fumarate), mirdametinib, pimasertib, refametinib, selumetinib (e.g., selumetinib sulfate), trametinib (e.g., trametinib dimethyl sulfoxide), zapnometinib, FCN-159, GSK-1120212, NFX-179, or TAK-733). In some embodiments, the cancer is a BRaf mutant NSCLC (e.g., BRaf V600E mutant NSCLC), and the additional therapy or therapeutic agent is dabrafenib (e.g., dabrafenib mesylate, GSK2118436), encorafenib (e.g., BRAFTOVI™, LGX818), or vemurafenib (e.g., ZELBORAF®, RO5185426), and binimetinib, cobimetinib (e.g., cobimetinib fumarate), selumetinib (e.g., selumetinib sulfate), or trametinib (e.g., trametinib dimethyl sulfoxide). In some embodiments, the cancer is a BRaf mutant NSCLC (e.g., BRaf V600E mutant NSCLC), and the additional therapy or therapeutic agent is dabrafenib (e.g., dabrafenib mesylate, GSK2118436) and trametinib (e.g., trametinib dimethyl sulfoxide). In some embodiments, the cancer is a BRaf mutant NSCLC (e.g., BRaf V600E mutant NSCLC), and the additional therapy or therapeutic agent is vemurafenib (e.g., ZELBORAF®, RO5185426), and cobimetinib (e.g., cobimetinib fumarate). In some embodiments, the cancer is a BRaf mutant NSCLC (e.g., BRaf V600E mutant NSCLC), and the additional therapy or therapeutic agent is encorafenib (e.g., BRAFTOVI™, LGX818) and binimetinib.

In some embodiments, the cancer is a BRaf mutant melanoma (e.g., BRaf V600E mutant melanoma), and the additional therapy or therapeutic agent is a BRaf inhibitor (e.g., avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, RO5185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, or PLX-4720). In some embodiments, the cancer is a BRaf mutant melanoma (e.g., BRaf V600E mutant melanoma), and the additional therapy or therapeutic agent is a BRaf inhibitor (e.g., avutometinib (RO5126766), dabrafenib (e.g., dabrafenib mesylate, GSK2118436), encorafenib (e.g., BRAFTOVI™, LGX818), naporafenib (LXH254), sorafenib (e.g., sorafenib tosylate), vemurafenib (e.g., ZELBORAF®, RO5185426), ARQ-736, AZ304, BMS-908662 (XL281), C17071479-F, CHIR-265 (RAF265), FORE-8394 (PLX-8394), GDC-0879, GDC-5573 (HM95573), HLX-208, PLX-3603, or PLX-4720) and a MEK inhibitor (e.g., avutometinib, binimetinib, cobimetinib (e.g., cobimetinib fumarate), mirdametinib, pimasertib, refametinib, selumetinib (e.g., selumetinib sulfate), trametinib (e.g., trametinib dimethyl sulfoxide), zapnometinib, FCN-159, GSK-1120212, NFX-179, or TAK-733). In some embodiments, the cancer is a BRaf mutant melanoma (e.g., BRaf V600E mutant melanoma), and the additional therapy or therapeutic agent is dabrafenib (e.g., dabrafenib mesylate, GSK2118436), encorafenib (e.g., BRAFTOVI™, LGX818), or vemurafenib (e.g., ZELBORAF®, RO5185426), and binimetinib, cobimetinib (e.g., cobimetinib fumarate), selumetinib (e.g., selumetinib sulfate), or trametinib (e.g., trametinib dimethyl sulfoxide). In some embodiments, the cancer is a BRaf mutant melanoma (e.g., BRaf V600E mutant melanoma), and the additional therapy or therapeutic agent is dabrafenib (e.g., dabrafenib mesylate, GSK2118436) and trametinib (e.g., trametinib dimethyl sulfoxide). In some embodiments, the cancer is a BRaf mutant melanoma (e.g., BRaf V600E mutant melanoma), and the additional therapy or therapeutic agent is vemurafenib (e.g., ZELBORAF®, RO5185426), and cobimetinib (e.g., cobimetinib fumarate). In some embodiments, the cancer is a BRaf mutant melanoma (e.g., BRaf V600E mutant melanoma), and the additional therapy or therapeutic agent is encorafenib (e.g., BRAFTOVI™, LGX818) and binimetinib. In some such embodiments, the subject has previously been treated with an immunotherapy.

In some embodiments, the CDK2 inhibitor is ebvaciclib, fadraciclib, milciclib, pacritinib (e.g., pacritinib citrate), roniciclib, roscovitine, BLU-222, NUV-422, PF-07104091, TQB-3616, or a combination thereof.

In some embodiments, the CDK4/6 inhibitor is abemaciclib, birociclib, dalpiciclib, lerociclib, milciclib, palbociclib, ribociclib (e.g., ribociclib succinate), riviciclib, roniciclib, trilaciclib (e.g., trilaciclib dihydrochloride), FCN-437, TQB-3616, or a combination thereof.

In some embodiments, the CDK7 inhibitor is milciclib, roscovitine, samuraciclib, or a combination thereof.

In some embodiments, the CDK9 inhibitor is fadraciclib, riviciclib, roniciclib, roscovitine, zotiraciclib, AZD-4573, KB-0742, or a combination thereof.

In some embodiments, the EGFR inhibitor is abivertinib, afatinib (e.g., afatinib dimaleate), alflutinib (e.g., alflutinib mesylate), almonertinib (e.g., almonertinib mesylate), befotertinib, brigatinib, canertinib, dacomitinib (e.g., dacomitinib monohydrate), dovitinib, erlotinib (e.g., erlotinib hydrochloride), gefitinib, icotinib, lapatinib (e.g., lapatinib ditosylate monohydrate), larotinib, lazertinib, limertinib, mobocertinib (e.g., mobocertinib succinate), nazartinib, neratinib (e.g., neratinib maleate), olmutinib, osimertinib (e.g., osimertinib mesylate), pelitinib, poziotinib, pyrotinib (e.g., pyrotinib maleate), ruserontinib (SKLB-1028), sapitinib, sunvozertinib, tesevatinib, vandetanib, varlitinib, zorifertinib, BIBW-2948, BPI-7711, HA-121-28, SH-1028, an anti-EGFR antibody or anti-EGFR antibody-drug conjugate, or a combination thereof.

In some embodiments, the anti-EGFR antibody or anti-EGFR antibody-drug conjugate is amivantamab (e.g., amivantamab-vmjw, or a biosimilar thereof), cetuximab (e.g., ERBITUX® (cetuximab), or a biosimilar thereof (e.g., CMAB-009, CPGJ-602, or KL-140)), cetuximab sarotalocan (AKALUX® (cetuximab sarotalocan), or a biosimilar thereof), depatuxizumab, duligotuzumab, futuximab, imgatuzumab, modotuximab, necitumumab (e.g., PORTRAZZA® (necitumumab), or a biosimilar thereof), nimotuzumab (e.g., BIOMAb EGFR® (nimotuzumab), or a biosimilar thereof), panitumumab (e.g., VECTIBIX® (panitumumab), or a biosimilar thereof), tomuzotuximab, zalutumumab, EMD-55900, EMD-82633, GC-1118, HLX-07, ICR-62, SCT-200, SI-B-001, TAS-0313, biosimilars thereof, or a combination thereof. In some embodiments, the anti-EGFR antibody or anti-EGFR antibody-drug conjugates is amivantamab (e.g., amivantamab-vmjw, or a biosimilar thereof), cetuximab (e.g., ERBITUX® (cetuximab), or a biosimilar thereof (e.g., CMAB-009, CPGJ-602, orKL-140)), cetuximab sarotalocan (AKALUX® (cetuximab sarotalocan), or a biosimilar thereof), depatuxizumab, duligotuzumab, futuximab, imgatuzumab, modotuximab, necitumumab (e.g., PORTRAZZA® (necitumumab), or a biosimilar thereof), nimotuzumab (e.g., BIOMAb EGFR® (nimotuzumab), or a biosimilar thereof), panitumumab (e.g., VECTIBIX® (panitumumab), or a biosimilar thereof), tomuzotuximab, zalutumumab, EMD-55900, EMD-82633, GC-1118, HLX-07, ICR-62, SCT-200, SI-B-001, biosimilars thereof, or a combination thereof.

In some embodiments, an EGFR mutation (e.g., an EGFR exon 19 deletion or an EGFR L858R mutation (with or without an EGFR T790M mutation)) can be detected in a sample from the subject (e.g., detecting an EGFR gene having a mutation (e.g., a mutation corresponding to an EGFR exon 19 deletion or an EGFR L858R mutation (with or without an EGFR T790M mutation) in an EGFR protein) and/or detecting an EGFR protein having a mutation (e.g., an EGFR exon 19 deletion or an EGFR L858R mutation (with or without an EGFR T790M mutation))). In some embodiments, the subject was determined (e.g., prior to administration of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a))) to have a cancer having an EGFR mutation (e.g., an EGFR exon 19 deletion or an EGFR L858R mutation (with or without an EGFR T790M mutation)).

In some embodiments, the cancer is an EGFR mutantNSCLC (e.g., EGFR exon 19 deletion NSCLC or EGFR L858R (with or without T790M) mutant NCLC), and the additional therapy or therapeutic agent is an EGFR inhibitor (e.g., abivertinib, afatinib (e.g., afatinib dimaleate), alflutinib (e.g., alflutinib mesylate), almonertinib (e.g., almonertinib mesylate), befotertinib, brigatinib, canertinib, dacomitinib (e.g., dacomitinib monohydrate), dovitinib, erlotinib (e.g., erlotinib hydrochloride), gefitinib, icotinib, lapatinib (e.g., lapatinib ditosylate monohydrate), larotinib, lazertinib, limertinib, mobocertinib (e.g., mobocertinib succinate), nazartinib, neratinib (e.g., neratinib maleate), olmutinib, osimertinib (e.g., osimertinib mesylate), pelitinib, poziotinib, pyrotinib (e.g., pyrotinib maleate), ruserontinib (SKLB-1028), sapitinib, sunvozertinib, tesevatinib, vandetanib, varlitinib, zorifertinib, BIBW-2948, BPI-7711, HA-121-28, SH-1028, or an anti-EGFR antibody or anti-EGFR antibody-drug conjugate). In some embodiments, the cancer is an EGFR mutant NSCLC (e.g., EGFR exon 19 deletion NSCLC or EGFR L858R (with or without T790M) mutant NCLC), and the additional therapy or therapeutic agent is osimertinib (e.g., osimertinib mesylate).

In some embodiments, the ERK inhibitor is rineterkib, temuterkib, ulixertinib, ASN-0007, ASTX-029, ATG-017, BPI-27336, HH-2710, JSI-1187, MK-8353, or a combination thereof.

In some embodiments, the EZH2 inhibitor is lirametostat, tazemetostat (e.g., tazemetostat hydrobromide), valemetostat (e.g., valemetostat tosylate), tulmimetostat (CPI-0209), EBI-2511, HH-2853, HM-97662, PF-6821497, SHR-2554, XNW-5004, or a combination thereof. In some embodiments, the EZH2 inhibitor also inhibits EZH1 (also referred to as an EZH1/2 inhibitor).

In some embodiments, the cancer is peripheral T cell lymphoma, and the additional therapy or therapeutic agent is an EZH2 inhibitor (e.g., lirametostat, tazemetostat (e.g., tazemetostat hydrobromide), valemetostat (e.g., valemetostat tosylate), tulmimetostat (CPI-0209), EBI-2511, HH-2853, HM-97662, PF-6821497, SHR-2554, or XNW-5004).

In some embodiments, the FGFR1 inhibitor is danusertib, dovitinib, erdafitinib, futibatinib, infigratinib (e.g., infigratinib phosphate), lenvatinib (e.g., lenvatinib mesylate), lucitanib, nintedanib (e.g., nintedanib esylate), pemigatinib, surufatinib, tasurgratinib, tinengotinib, zoligratinib, FH-2001, HMPL-453, LY-2874455, or a combination thereof.

In some embodiments, the FGFR2 inhibitor is erdafitinib, futibatinib, infigratinib (e.g., infigratinib phosphate), lucitanib, pemigatinib, tasurgratinib, zoligratinib, bemarituzumab (or biosimilars thereof), FH-2001, HMPL-453, LY-2874455, or a combination thereof.

In some embodiments, the FGFR3 inhibitor is dovitinib, erdafitinib, futibatinib, infigratinib (e.g., infigratinib phosphate), lucitanib, masitinib, nintedanib, pemigatinib, tasurgratinib, zoligratinib, vofatamab (or biosimilars thereof), EXEL-0999, FH-2001, HMPL-453, LY-2874455, or a combination thereof.

In some embodiments, the FGFR4 inhibitor is axitinib, erdafitinib, futibatinib, infigratinib (e.g., infigratinib phosphate), irpagratinib, nintedanib, pemigatinib, FH-2001, H3B-6527, LY-2874455, or a combination thereof.

In some embodiments, the HER2 inhibitor is afatinib (e.g., afatinib dimaleate), dacomitinib (e.g., dacomitinib monohydrate), lapatinib (e.g., lapatinib ditosylate monohydrate), mobocertinib (e.g., mobocertinib succinate), neratinib (e.g., neratinib maleate), poziotinib, pyrotinib (e.g., pyrotinib maleate), sunvozertinib, tesevatinib, tucatinib, varlitinib, an anti-HER2 antibody or anti-HER2 antibody-drug conjugate, or a combination thereof.

In some embodiments, the anti-HER2 antibody or anti-HER2 antibody-drug conjugate is anbenitamab, cinrebafusp alfa, coprelotamab, disitamab vedotin, ertumaxomab, gancotamab, inetetamab, margetuximab (e.g., margetuximab-cmkb, or a biosimilar thereof), pertuzumab (e.g., PERJETA® (pertuzumab), or a biosimilar thereof (e.g., HLX-11)), trastuzumab (e.g., HERCEPTIN® (trastuzumab), or a biosimilar thereof (e.g., FACEPTOR® (trastuzumab), HERTICAD® (trastuzumab), TUZNUE® (trastuzumab), ZERCEPAC® (trastuzumab), trastuzumab-anns, trastuzumab-dkst, trastuzumab-dttb, trastuzumab-pkrb, trastuzumab-qyyp, EG-12014, or TX-05)), trastuzumab deruxtecan (e.g., fam-trastuzumab deruxtecan-nxki, or a biosimilar thereof), trastuzumab duocarmazine, trastuzumab emtansine (e.g., KADCYLA® (trastuzumab emtansine), or a biosimilar thereof (e.g., UJVIRA® (trastuzumab emtansine))), trastuzumab hyaluronidase (e.g., trastuzumab hyaluronidase-oysk, or a biosimilar thereof), zanidatamab, zenocutuzumab, AVX-901, IDM-1, TPIV-100, TAA-013, SHR-A1811, BAT-8001, MDX-210, Alpha-Her2-pAF1-AS-269, MRG-002, DF-1001, AC-101, MM-111, biosimilars thereof, or a combination thereof. In some embodiments, the anti-HER2 antibody or anti-HER2 antibody-drug conjugate is anbenitamab, cinrebafusp alfa, coprelotamab, disitamab vedotin, ertumaxomab, gancotamab, inetetamab, margetuximab (e.g., margetuximab-cmkb, or a biosimilar thereof), pertuzumab (e.g., PERJETA® (pertuzumab), or a biosimilar thereof (e.g., HLX-11)), trastuzumab (e.g., HERCEPTIN® (trastuzumab), or a biosimilar thereof (e.g., FACEPTOR® (trastuzumab), HERTICAD® (trastuzumab), TUZNUE® (trastuzumab), ZERCEPAC® (trastuzumab), trastuzumab-anns, trastuzumab-dkst, trastuzumab-dttb, trastuzumab-pkrb, trastuzumab-qyyp, EG-12014, or TX-05)), trastuzumab deruxtecan (e.g., fam-trastuzumab deruxtecan-nxki, or a biosimilar thereof), trastuzumab duocarmazine, trastuzumab emtansine (e.g., KADCYLA® (trastuzumab emtansine), or a biosimilar thereof (e.g., UJVIRA® (trastuzumab emtansine))), trastuzumab hyaluronidase (e.g., trastuzumab hyaluronidase-oysk, or a biosimilar thereof), zanidatamab, zenocutuzumab, TAA-013, SHR-A1811, BAT-8001, MDX-210, Alpha-Her2-pAF1-AS-269, MRG-002, DF-1001, AC-101, MM-111, biosimilars thereof, or a combination thereof.

In some embodiments, HER2+ status can be detected in a sample from the subject (e.g., via immunohistochemistry (IHC) and/or fluorescent in situ hybridization (FISH)). In some embodiments, the subject was determined (e.g., prior to administration of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a))) to have a cancer with HER2+ status.

In some embodiments, HER2 low status can be detected in a sample from the subject (e.g., via IHC and/or FISH). In some embodiments, the subject was determined (e.g., prior to administration of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a))) to have a cancer with HER2 low status.

In some embodiments, HER2− status can be detected in a sample from the subject (e.g., via IHC and/or FISH). In some embodiments, the subject was determined (e.g., prior to administration of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a))) to have a cancer with HER2− status.

In some embodiments, ER expression status can be detected in a sample from the subject (e.g., via IHC and/or FISH). In some embodiments, the subject was determined (e.g., prior to administration of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a))) to have a cancer with ER expression. In some embodiments, the subject was determined (e.g., prior to administration of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a))) to have a cancer without ER expression.

In some embodiments, PR expression status can be detected in a sample from the subject (e.g., via IHC, and/or FISH). In some embodiments, the subject was determined (e.g., prior to administration of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a))) to have a cancer with PR expression. In some embodiments, the subject was determined (e.g., prior to administration of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a))) to have a cancer without PR expression.

In some embodiments, the cancer is a HER2+ breast cancer (e.g., HER2+ breast cancer with ER expression, HER2+ breast cancer without ER expression), and the additional therapy is a HER2 inhibitor (e.g., afatinib (e.g., afatinib dimaleate), dacomitinib (e.g., dacomitinib monohydrate), lapatinib (e.g., lapatinib ditosylate monohydrate), mobocertinib (e.g., mobocertinib succinate), neratinib (e.g., neratinib maleate), poziotinib, pyrotinib (e.g., pyrotinib maleate), sunvozertinib, tesevatinib, tucatinib, varlitinib, or an anti-HER2 antibody or anti-HER2 antibody-drug conjugate). In some embodiments, the cancer is a HER2+ breast cancer (e.g., HER2+ breast cancer with ER expression, HER2+ breast cancer without ER expression), and the additional therapy is tucatinib. In some embodiments, the cancer is a HER2+ breast cancer (e.g., HER2+ breast cancer with ER expression, HER2+ breast cancer without ER expression), and the additional therapy is an anti-HER2 antibody or anti-HER2 antibody-drug conjugate (e.g., anbenitamab, cinrebafusp alfa, coprelotamab, disitamab vedotin, ertumaxomab, gancotamab, inetetamab, margetuximab (e.g., margetuximab-cmkb, or a biosimilar thereof), pertuzumab (e.g., PERJETA® (pertuzumab), or a biosimilar thereof (e.g., HLX-11)), trastuzumab (e.g., HERCEPTIN® (trastuzumab), or a biosimilar thereof (e.g., FACEPTOR® (trastuzumab), HERTICAD® (trastuzumab), TUZNUE® (trastuzumab), ZERCEPAC® (trastuzumab), trastuzumab-anns, trastuzumab-dkst, trastuzumab-dttb, trastuzumab-pkrb, trastuzumab-qyyp, EG-12014, or TX-05)), trastuzumab deruxtecan (e.g., fam-trastuzumab deruxtecan-nxki, or a biosimilar thereof), trastuzumab duocarmazine, trastuzumab emtansine (e.g., KADCYLA® (trastuzumab emtansine), or a biosimilar thereof (e.g., UJVIRA® (trastuzumab emtansine))), trastuzumab hyaluronidase (e.g., trastuzumab hyaluronidase-oysk, or a biosimilar thereof), zanidatamab, zenocutuzumab, AVX-901, IDM-1, TPIV-100, TAA-013, SHR-A1811, BAT-8001, MDX-210, Alpha-Her2-pAF1-AS-269, MRG-002, DF-1001, AC-101, MM-111, or biosimilars thereof). In some embodiments, the cancer is a HER2+ breast cancer (e.g., HER2+ breast cancer with ER expression, HER2+ breast cancer without ER expression), and the additional therapy is trastuzumab (e.g., HERCEPTIN® (trastuzumab), or a biosimilar thereof (e.g., FACEPTOR® (trastuzumab), HERTICAD® (trastuzumab), TUZNUE® (trastuzumab), ZERCEPAC® (trastuzumab), trastuzumab-anns, trastuzumab-dkst, trastuzumab-dttb, trastuzumab-pkrb, trastuzumab-qyyp, EG-12014, or TX-05)). In some embodiments, the cancer is a HER2+ breast cancer (e.g., HER2+ breast cancer with ER expression, HER2+ breast cancer without ER expression), and the additional therapy is trastuzumab deruxtecan (e.g., fam-trastuzumab deruxtecan-nxki, or a biosimilar thereof). In some embodiments, the cancer is a HER2+ breast cancer (e.g., HER2+ breast cancer with ER expression, HER2+ breast cancer without ER expression), and the additional therapy is trastuzumab emtansine, or a biosimilar thereof. In some embodiments, the cancer is a HER2 low breast cancer, and the additional therapy is an anti-HER2 antibody or anti-HER2 antibody-drug conjugate. In some embodiments, the cancer is a HER2 low breast cancer, and the additional therapy is trastuzumab deruxtecan (e.g., fam-trastuzumab deruxtecan-nxki, or a biosimilar thereof).

In some embodiments, the JAK inhibitor is adelatinib, baricitinib, brepocitinib, deuruxolitinib, fedratinib (e.g., fedratinib dihydrochloride monohydrate), filgotinib (e.g., filgotinib maleate), gandotinib, gusacitinib, ilginatinib, izencitinib, jaktinib, momelotinib (e.g., momelotinib dihydrochloride), nezulcitinib, pacritinib (e.g., pacritinib citrate), peficitinib (e.g., peficitinib hydrobromide), ropsacitinib, ruxolitinib (e.g., ruxolitinib phosphate), tasocitinib (e.g., tofacitinib citrate), AT-9283, TQ-05105, or a combination thereof. In some embodiments, the JAK inhibitor is fedratinib (e.g., fedratinib dihydrochloride monohydrate), momelotinib (e.g., momelotinib dihydrochloride), pacritinib (e.g., pacritinib citrate), ruxolitinib (e.g., ruxolitinib phosphate), or a combination thereof. In some embodiments, the JAK inhibitor is fedratinib (e.g., fedratinib dihydrochloride monohydrate). In some embodiments, the JAK inhibitor is momelotinib (e.g., momelotinib dihydrochloride). In some embodiments, the JAK inhibitor is pacritinib (e.g., pacritinib citrate). In some embodiments, the JAK inhibitor is ruxolitinib (e.g., ruxolitinib phosphate).

In some embodiments, a JAK V617F mutation can be detected in a sample from the subject (e.g., detecting a JAK2 gene having a mutation corresponding to a V617F mutation in JAK2 protein and/or detecting a JAK2 protein having a V617F mutation). In some embodiments, the subject was determined (e.g., prior to administration of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a))) to have a cancer having a JAK2 V617F mutation. In some embodiments, the subject was determined (e.g., prior to administration of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (I-a))) to have a cancer lacking a JAK2 V617F mutation.

In some embodiments, the cancer is post-MPN AML, M6-AML, or M7-AML, and the additional therapy or therapeutic agent is a JAK inhibitor (e.g., ruxolitinib (e.g., ruxolitinib phosphate), fedratinib (e.g., fedratinib dihydrochloride monohydrate), momelotinib (e.g., momelotinib dihydrochloride), or pacritinib (e.g., pacritinib citrate)). In some embodiments, the patient has received a previous line of therapy including a JAK inhibitor. In some embodiments, the patient has not received a previous line of therapy including a JAK inhibitor.

In some embodiments, the cancer is intermediate (e.g., intermediate-1 and/or intermediate-2) or high-risk myelofibrosis (e.g., primary myelofibrosis, post-essential thrombocythemia myelofibrosis, or post-polycythemia vera myelofibrosis), and the additional therapy or therapeutic agent is a JAK inhibitor (e.g., ruxolitinib (e.g., ruxolitinib phosphate), fedratinib (e.g., fedratinib dihydrochloride monohydrate), momelotinib (e.g., momelotinib dihydrochloride), or pacritinib (e.g., pacritinib citrate)). In some embodiments, the cancer is intermediate (e.g., intermediate-1 and/or intermediate-2) or high-risk myelofibrosis (e.g., primary myelofibrosis, post-essential thrombocythemia myelofibrosis, or post-polycythemia vera myelofibrosis), and the additional therapy or therapeutic agent is a JAK inhibitor (e.g., ruxolitinib (e.g., ruxolitinib phosphate), fedratinib (e.g., fedratinib dihydrochloride monohydrate), momelotinib (e.g., momelotinib dihydrochloride), or pacritinib (e.g., pacritinib citrate)) and a BET inhibitor (e.g., alobresib, apabetalone, mivebresib, pelabresib, trotabresib, ABBV-744, BI-2536, BMS-986158, INCB-057643, JAB-8263, ODM-207, PLX-2853, or ZEN-003694). In some embodiments, the cancer is intermediate (e.g., intermediate-1 and/or intermediate-2) or high-risk myelofibrosis (e.g., primary myelofibrosis, post-essential thrombocythemia myelofibrosis, or post-polycythemia vera myelofibrosis) with a JAK2 mutation (e.g., a JAK2 V617F mutation), and the additional therapy or therapeutic agent is a JAK inhibitor (e.g., ruxolitinib (e.g., ruxolitinib phosphate), fedratinib (e.g., fedratinib dihydrochloride monohydrate), momelotinib (e.g., momelotinib dihydrochloride), or pacritinib (e.g., pacritinib citrate)). In some embodiments, the cancer is intermediate (e.g., intermediate-1 and/or intermediate-2) or high-risk myelofibrosis (e.g., primary myelofibrosis, post-essential thrombocythemia myelofibrosis, or post-polycythemia vera myelofibrosis) with a JAK2 mutation (e.g., a JAK2 V617F mutation), and the additional therapy or therapeutic agent is a JAK inhibitor (e.g., ruxolitinib (e.g., ruxolitinib phosphate), fedratinib (e.g., fedratinib dihydrochloride monohydrate), momelotinib (e.g., momelotinib dihydrochloride), or pacritinib (e.g., pacritinib citrate)) and a BET inhibitor (e.g., alobresib, apabetalone, mivebresib, pelabresib, trotabresib, ABBV-744, BI-2536, BMS-986158, INCB-057643, JAB-8263, ODM-207, PLX-2853, or ZEN-003694). In some embodiments, the JAK inhibitor is fedratinib (e.g., fedratinib dihydrochloride monohydrate). In some embodiments, the JAK inhibitor is momelotinib (e.g., momelotinib dihydrochloride). In some embodiments, the JAK inhibitor is pacritinib (e.g., pacritinib citrate). In some embodiments, the JAK inhibitor is ruxolitinib (e.g., ruxolitinib phosphate). In some embodiments, the patient has received a previous line of therapy including a JAK inhibitor. In some embodiments, the patient has not received a previous line of therapy including a JAK inhibitor. In some embodiments, treatment effect can be measured by Spleen Volume Reduction (e.g., Spleen Volume Reduction of greater than or equal to 35% (SVR₃₅), for example, measured by MRI or CT), Total Symptom Score (e.g., Total Symptom Score reduction of greater than or equal to 50% (TSS₅₀), for example, measured by the Myelofibrosis Symptom Assessment Form (MFSAF) version 4.0), or both, such as at 24 weeks after beginning of treatment; see, e.g., Harrison, Claire, et al., New England Journal of Medicine 366.9 (2012): 787-798; and Verstovsek, Srdan, et al. New England Journal of Medicine 366.9 (2012): 799-807. In some embodiments, treatment effect can be measured (e.g., in addition to or instead of SVR₃₅ and/or TSS₅₀) by anemia response (e.g., measured by current International Working Group-Myeloproliferative Neoplasms Research and European LeukemiaNet (IWG-MRT/ELN) criteria), bone marrow fibrosis (e.g., according to the European Consensus Grading System through bone marrow biopsy, such as at 24 or 96 weeks after beginning of treatment), variant allele fraction (e.g., JAK2 V617F variant allele fraction), transfusion independence, overall survival, leukemia-free survival, change in physical functioning (e.g., measured by the physical functioning domain of the European Organization for Research and Treatment of Cancer (EORTC) Quality of Life Questionnaire (QLQ)-C30 or death), change in fatigue (e.g., assessed using the Patient-Reported Outcomes Measurement Information System (PROMIS) Fatigue SF 7a), or a combination thereof.

In some embodiments, the KRas inhibitor is adagrasib, divarasib (GDC-6036), sotorasib, ARS-1620, ARS-3248, ARS-853, ASP-3082, ATG-012, BI-1701963, BI-1823911, BPI-421286, D-1553, ERAS-3490, GFH-925, JAB-21822, JDQ-443, LY-3537982, MRTX-1133, MRTX-1257, RMC-6236, RMC-6291, RSC-1255, or a combination thereof.

In some embodiments, a KRas mutation (e.g., a KRas G12C mutation or a KRas G12D mutation) can be detected in a sample from the subject (e.g., detecting a KRAS gene having a mutation corresponding to a G12C mutation or a G12D mutation in KRas protein and/or detecting a KRas protein having a G12C mutation or a G12D mutation). In some embodiments, the subject was determined (e.g., prior to administration of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a))) to have a cancer having a KRas G12C mutation. In some embodiments, the subject was determined (e.g., prior to administration of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a))) to have a cancer having a KRas G12D mutation.

In some embodiments, the cancer is a KRas mutant lung cancer (e.g., a KRas mutant NSCLC), a KRas mutant CRC, or a KRas mutant pancreatic cancer, and the additional therapy or therapeutic agent is a KRas inhibitor (e.g., adagrasib, divarasib (GDC-6036), sotorasib, ARS-1620, ARS-3248, ARS-853, ASP-3082, ATG-012, BI-1701963, BI-1823911, BPI-421286, D-1553, ERAS-3490, GFH-925, JAB-21822, JDQ-443, LY-3537982, MRTX-1133, MRTX-1257, RMC-6236, RMC-6291, or RSC-1255). In some embodiments, the cancer is a KRas mutant lung cancer (e.g., a KRas mutant NSCLC (e.g., a KRas G12C mutant NSCLC)), a KRas mutant CRC (e.g., a KRas G12C mutant CRC), or a KRas mutant pancreatic cancer (e.g., a KRas G12C mutant pancreatic cancer), and the additional therapy or therapeutic agent is adagrasib. In some embodiments, the cancer is a KRas mutant lung cancer (e.g., a KRas mutant NSCLC (e.g., a KRas G12C mutant NSCLC)), a KRas mutant CRC (e.g., a KRas G12C mutant CRC), or a KRas mutant pancreatic cancer (e.g., a KRas G12C mutant pancreatic cancer), and the additional therapy or therapeutic agent is divarasib. In some embodiments, the cancer is a KRas mutant lung cancer (e.g., a KRas mutant NSCLC (e.g., a KRas G12C mutant NSCLC)), a KRas mutant CRC (e.g., a KRas G12C mutant CRC), or a KRas mutant pancreatic cancer (e.g., a KRas G12C mutant pancreatic cancer), and the additional therapy or therapeutic agent is sotorasib. In some embodiments, the cancer is a KRas mutant lung cancer (e.g., a KRas mutant NSCLC (e.g., a KRas G12D mutant NSCLC)), a KRas mutant CRC (e.g., a KRas G12D mutant CRC), or a KRas mutant pancreatic cancer (e.g., a KRas G12D mutant pancreatic cancer), and the additional therapy or therapeutic agent is MRTX1133.

In some embodiments, the MEK inhibitor is avutometinib, binimetinib, cobimetinib (e.g., cobimetinib fumarate), mirdametinib, pimasertib, refametinib, selumetinib (e.g., selumetinib sulfate), trametinib (e.g., trametinib dimethyl sulfoxide), zapnometinib, FCN-159, GSK-1120212, NFX-179, TAK-733, or a combination thereof.

In some embodiments, a BRCA1 mutation can be detected in a sample from the subject (e.g., detecting a BRCA1 gene having a mutation and/or detecting a BRCA1 protein having a mutation). In some embodiments, the subject was determined (e.g., prior to administration of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a))) to have a cancer having a BRCA1 mutation.

In some embodiments, a BRCA2 mutation can be detected in a sample from the subject (e.g., detecting a BRCA2 gene having a mutation and/or detecting a BRCA2 protein having a mutation). In some embodiments, the subject was determined (e.g., prior to administration of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a))) to have a cancer having a BRCA2 mutation.

In some embodiments, the cancer is ovarian cancer (e.g., BRCA1 mutant ovarian cancer or BRCA2 mutant ovarian cancer, HGSOC (e.g., BRCA1 mutant HGSOC or BRCA2 mutant HGSOC), or LGSOC), and the additional therapy or therapeutic agent is a MEK inhibitor (e.g., binimetinib, cobimetinib, selumetinib, or trametinib). In some embodiments, the cancer is BRCA1 mutant ovarian cancer, and the additional therapy or therapeutic agent is a MEK inhibitor (e.g., binimetinib, cobimetinib, selumetinib, or trametinib). In some embodiments, the cancer is BRCA2 mutant ovarian cancer, and the additional therapy or therapeutic agent is a MEK inhibitor (e.g., binimetinib, cobimetinib, selumetinib, or trametinib). In some embodiments, the cancer is HGSOC (e.g., BRCA1 mutant HGSOC or BRCA2 mutant HGSOC), and the additional therapy or therapeutic agent is a MEK inhibitor (e.g., binimetinib, cobimetinib, selumetinib, or trametinib). In some embodiments, the cancer is LGSOC (e.g., BRCA1 mutant HGSOC or BRCA2 mutant HGSOC), and the additional therapy or therapeutic agent is a MEK inhibitor (e.g., binimetinib, cobimetinib, selumetinib, or trametinib).

In some embodiments, the cancer is a KRas mutant CRC (e.g., a KRas G12C mutant NSCLC), and the additional therapy or therapeutic agent is a MEK inhibitor (e.g., binimetinib, cobimetinib, selumetinib, or trametinib).

In some embodiments, the MET inhibitor is cabozantinib (e.g., cabozantinib S-malate), capmatinib (e.g., capmatinib hydrochloride), crizotinib, foritinib, glesatinib, gumarontinib, merestinib, pamufetinib, savolitinib, sitravatinib, tepotinib (e.g., tepotinib hydrochloride hydrate), vebreltinib, zanzalintinib (XL-092), amivantamab (e.g., amivantamab-vmjw, or a biosimilar thereof), emibetuzumab (or biosimilars thereof), RC-108, telisotuzumab vedotin (or biosimilars thereof), ABBV-400, ABN-401, AL-2846, AMG-337, SAR-125844, TQ-B-3139, or a combination thereof.

In some embodiments, a MET alteration can be detected in a sample from the subject (e.g., detecting a MET gene having an alteration (e.g., gene amplification or exon14 skipping) and/or detecting a MET protein having a mutation (e.g., exon14 skipping)). In some embodiments, the subject was determined (e.g., prior to administration of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a))) to have a cancer having a MET alteration.

In some embodiments, the cancer is a MET-altered NCLC (e.g., MET amplified NSCLC or MET exon14 skipping NSCLC), and the additional therapy or therapeutic agent is a MET inhibitor (e.g., cabozantinib (e.g., cabozantinib S-malate), capmatinib (e.g., capmatinib hydrochloride), crizotinib, foritinib, glesatinib, gumarontinib, merestinib, pamufetinib, savolitinib, sitravatinib, tepotinib (e.g., tepotinib hydrochloride hydrate), vebreltinib, zanzalintinib (XL-092), amivantamab (e.g., amivantamab-vmjw, or a biosimilar thereof), emibetuzumab (or biosimilars thereof), RC-108, telisotuzumab vedotin (or biosimilars thereof), ABBV-400, ABN-401, AL-2846, AMG-337, SAR-125844, or TQ-B-3139). In some embodiments, the cancer is a MET-altered NCLC (e.g., MET amplified NSCLC or MET exon14 skipping NSCLC), and the additional therapy or therapeutic agent is capmatinib (e.g., capmatinib hydrochloride) or tepotinib (e.g., tepotinib hydrochloride hydrate). In some embodiments, the cancer is a MET-altered NCLC (e.g., MET amplified NSCLC or MET exon14 skipping NSCLC), and the additional therapy or therapeutic agent is telisotuzumab vedotin (or biosimilars thereof).

In some embodiments, the Hif2α inhibitor is belzutifan, AB-521, DFF-332, NKT-2152, PT-2399, or a combination thereof.

In some embodiments, the PARP inhibitor is fuzuloparib (fluzoparib), niraparib (e.g., niraparib tosylate monohydrate), olaparib, pamiparib, rucaparib (e.g., rucaparib camsylate), saruparib (AZD5305), senaparib, stenoparib, talazoparib (e.g., talazoparib tosylate), veliparib, CEP-9722, JPI-289, NMS-03305293, or a combination thereof. In some embodiments, the PARP inhibitor is a PARP1 inhibitor. In some embodiments, the PARP1 inhibitor is saruparib (AZD5305), NMS-03305293, or a combination thereof.

In some embodiments, the cancer is BRCA1 mutant breast cancer or BRCA2 mutant breast cancer, and the additional therapy or therapeutic agent is a PARP inhibitor (e.g., fuzuloparib (fluzoparib), niraparib (e.g., niraparib tosylate monohydrate), olaparib, pamiparib, rucaparib (e.g., rucaparib camsylate), saruparib (AZD5305), senaparib, stenoparib, talazoparib (e.g., talazoparib tosylate), veliparib, CEP-9722, JPI-289, or NMS-03305293). In some embodiments, the cancer is BRCA1 mutant breast cancer or BRCA2 mutant breast cancer, and the additional therapy or therapeutic agent is saruparib.

In some embodiments, the cancer is triple negative breast cancer, and the additional therapy or therapeutic agent is a PARP inhibitor (e.g., fuzuloparib (fluzoparib), niraparib (e.g., niraparib tosylate monohydrate), olaparib, pamiparib, rucaparib (e.g., rucaparib camsylate), saruparib (AZD5305), senaparib, stenoparib, talazoparib (e.g., talazoparib tosylate), veliparib, CEP-9722, JPI-289, or NMS-03305293). In some embodiments, the cancer is triple negative breast cancer, and the additional therapy or therapeutic agent is saruparib.

In some embodiments, the cancer is HGSOC (e.g., BRCA1 mutant HGSOC or BRCA2 mutant HGSOC), and the additional therapy or therapeutic agent is a PARP inhibitor (e.g., fuzuloparib (fluzoparib), niraparib (e.g., niraparib tosylate monohydrate), olaparib, pamiparib, rucaparib (e.g., rucaparib camsylate), saruparib (AZD5305), senaparib, stenoparib, talazoparib (e.g., talazoparib tosylate), veliparib, CEP-9722, JPI-289, or NMS-03305293). In some embodiments, the cancer is HGSOC (e.g., BRCA1 mutant HGSOC or BRCA2 mutant HGSOC), and the additional therapy or therapeutic agent is saruparib.

In some embodiments, the LSD1 inhibitor is bomedemstat, iadademstat, pulrodemstat, seclidemstat (HCI-2577), vafidemstat, GSK-2879552, INCB-059872, JBI-802, or a combination thereof.

In some embodiments, the BET inhibitor is alobresib, apabetalone, mivebresib, pelabresib, trotabresib, ABBV-744, BI-2536, BMS-986158, INCB-057643, JAB-8263, ODM-207, PLX-2853, ZEN-003694, or a combination thereof.

In some embodiments, the cancer is myelofibrosis (e.g., primary myelofibrosis, post-essential thrombocythemia myelofibrosis, or post-polycythemia vera myelofibrosis), and the additional therapy or therapeutic agent is a BET inhibitor (e.g., alobresib, apabetalone, mivebresib, pelabresib, trotabresib, ABBV-744, BI-2536, BMS-986158, INCB-057643, JAB-8263, ODM-207, PLX-2853, or ZEN-003694).

In some embodiments, the STING agonist is ulevostinag, ADU-S100, or a combination thereof.

In some embodiments, the telomerase inhibitor is imetelstat (e.g., imetelstat sodium).

In some embodiments, the TORC1/2 inhibitor is apitolisib, bimiralisib, dactolisib, deforolimus, everolimus, fosciclopirox (e.g., fosciclopirox sodium), gedatolisib, onatasertib, paxalisib, sapanisertib, sirolimus, sodium 2-hydroxylinoleate, temsirolimus, umirolimus, vistusertib, zandelisib, zotarolimus, BI-860585, CC-115, CLL-442, PF-04691502, or a combination thereof.

In some embodiments, the VEGFR inhibitor is apatinib, axitinib, cabozantinib (e.g., cabozantinib S-malate), catequentinib (alontinib), cediranib, dovitinib, famitinib, fruquintinib, glesatinib, ibcasertib, ilorasertib, lenvatinib (e.g., lenvatinib mesylate), lucitanib, nintedanib (e.g., nintedanib esylate), pamufetinib, pazopanib (e.g., pazopanib hydrochloride), regorafenib (e.g., regorafenib monohydrate), sitravatinib, sorafenib (e.g., sorafenib tosylate), sunitinib (e.g., sunitinib malate), surufatinib (sulfatinib), telatinib, tinengotinib, tivozanib (e.g., tivozanib hydrochloride monohydrate), vandetanib, vorolanib, zanzalintinib, olinvacimab (or biosimilars thereof), ramucirumab (or biosimilars thereof), CEP-11981, ENMD-2076, ODM-203, or a combination thereof.

In some embodiments, the chemotherapy is a platinum complex, a microtubule inhibitor (e.g., a microtubule destabilizer or a microtubule stabilizer), a topoisomerase inhibitor, a hypomethylating agent, or an antibody-drug conjugate including any thereof. In some embodiments, the platinum complex is carboplatin, cisplatin, lobaplatin, miriplatin, oxaliplatin, or a combination thereof. In some embodiments, the microtubule inhibitor is cabazitaxel, colchicine, desoxyepothilone B, docetaxel, eribulin, ixabepilone, nab-paclitaxel, paclitaxel, plinabulin, sabizabulin, tirbanibulin, vinblastine, vinflunine, vinorelbine, or a combination thereof. In some embodiments, the microtubule inhibitor is cabazitaxel, docetaxel, nab-paclitaxel, paclitaxel, or a combination thereof. In some embodiments, the topoisomerase inhibitor is aclarubicin, amsacrine, belotecan, camptothecin, daunorubicin, dexrazoxane, elliptinium, epirubicin, etoposide, gepotidacin, idarubicin, mitoxantrone, nemonoxacin, pirarubicin, pixantrone, razoxane, rubitecan, sobuzoxane, temozolomide, teniposide, topotecan, SN-38, or a combination thereof. In some embodiments, the hypomethylating agent is azacitidine, decitabine, or a combination thereof. In some embodiments, the chemotherapy is a platinum complex and a topoisomerase inhibitor (e.g., cisplatin and etoposide). In some embodiments, the antibody-drug conjugate including the microtubule inhibitor is belantamab mafodotin, brentuximab vedotin, cofetuzumab pelidotin, disitamab vedotin, enfortumab vedotin (e.g., enfortumab vedotin-ejfv, or a biosimilar thereof), mirvetuximab soravtansine (e.g., mirvetuximab soravtansine-gynx, or a biosimilar thereof), polatuzumab vedotin, telisotuzumab vedotin, tisotumab vedotin, trastuzumab emtansine (e.g., ado-trastuzumab emtansine, or a biosimilar thereof), tusamitamab ravtansine, upifitamab rilsodotin, zilovertamab vedotin, Alpha-Her2-pAF1-AS-269, BAT-8001, TAA-013, biosimilars thereof, or a combination thereof. In some embodiments, the antibody-drug conjugate including the microtubule inhibitor is enfortumab vedotin (e.g., enfortumab vedotin-ejfv, or a biosimilar thereof). In some embodiments, the antibody-drug conjugate including the microtubule inhibitor is mirvetuximab soravtansine (e.g., mirvetuximab soravtansine-gynx, or a biosimilar thereof). In some embodiments, the antibody-drug conjugate including the microtubule inhibitor is trastuzumab emtansine (e.g., ado-trastuzumab emtansine, or a biosimilar thereof). In some embodiments, the antibody-drug conjugate including the topoisomerase inhibitor is datopotamab deruxtecan, patritumab deruxtecan, sacituzumab govitecan (e.g., sacituzumab govitecan-hziy, or a biosimilar thereof), trastuzumab deruxtecan (fam-trastuzumab deruxtecan-nxki, or a biosimilar thereof), or a combination thereof. In some embodiments, the antibody-drug conjugate including the topoisomerase inhibitor is sacituzumab govitecan (e.g., sacituzumab govitecan-hziy, or a biosimilar thereof). In some embodiments, the antibody-drug conjugate including the topoisomerase inhibitor is trastuzumab deruxtecan (e.g., fam-trastuzumab deruxtecan-nxki, or a biosimilar thereof).

In some embodiments, the cancer is a lung cancer (e.g., NSCLC (e.g., squamous cell carcinoma)), and the additional therapy or therapeutic agent is a microtubule inhibitor (e.g., cabazitaxel, docetaxel, nab-paclitaxel, or paclitaxel). In some embodiments, the cancer is a lung cancer (e.g., NSCLC (e.g., squamous cell carcinoma)), and the additional therapy or therapeutic agent is docetaxel.

In some embodiments, the cancer is NSCLC (e.g., NSCLC with a MET amplification), and the additional therapy or therapeutic agent is telisotuzumab vedotin.

In some embodiments, the cancer is a lung cancer (e.g., NSCLC), and the additional therapy or therapeutic agent is a platinum complex (e.g., carboplatin, cisplatin, lobaplatin, miriplatin, or oxaliplatin) and anti-PD1 therapy.

In some embodiments, the cancer is a lung cancer (e.g., NSCLC), and the additional therapy or therapeutic agent is a platinum complex (e.g., carboplatin, cisplatin, lobaplatin, miriplatin, or oxaliplatin) and anti-PD-L1 therapy.

In some embodiments, the cancer is a lung cancer (e.g., NSCLC), and the additional therapy or therapeutic agent is a pemetrexed and anti-PD1 therapy.

In some embodiments, the cancer is a lung cancer (e.g., NSCLC), and the additional therapy or therapeutic agent is a pemetrexed and anti-PD-L1 therapy.

In some embodiments, the cancer is a lung cancer (e.g., SCLC), and the additional therapy or therapeutic agent is a platinum complex (e.g., carboplatin, cisplatin, lobaplatin, miriplatin, or oxaliplatin) and a topoisomerase inhibitor (e.g., aclarubicin, amsacrine, belotecan, camptothecin, daunorubicin, dexrazoxane, elliptinium, epirubicin, etoposide, gepotidacin, idarubicin, mitoxantrone, nemonoxacin, pirarubicin, pixantrone, razoxane, rubitecan, sobuzoxane, temozolomide, teniposide, topotecan, or SN-38). In some embodiments, the cancer is a lung cancer (e.g., SCLC), and the additional therapy or therapeutic agent is carboplatin and etoposide. In some embodiments, the cancer is a lung cancer (e.g., SCLC), and the additional therapy or therapeutic agent is cisplatin and etoposide.

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), and the additional therapy or therapeutic agent is a microtubule inhibitor (e.g., cabazitaxel, docetaxel, nab-paclitaxel, or paclitaxel). In some embodiments, the cancer is triple negative breast cancer, and the additional therapy or therapeutic agent is a microtubule inhibitor (e.g., cabazitaxel, docetaxel, nab-paclitaxel, or paclitaxel). In some embodiments, the cancer is triple negative breast cancer, and the additional therapy or therapeutic agent is nab-paclitaxel or paclitaxel.

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), and the additional therapy or therapeutic agent is a microtubule inhibitor (e.g., cabazitaxel, docetaxel, nab-paclitaxel, or paclitaxel) and anti-PD1 therapy.

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), and the additional therapy or therapeutic agent is a microtubule inhibitor (e.g., cabazitaxel, docetaxel, nab-paclitaxel, or paclitaxel) and anti-PD-L1 therapy.

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), and the additional therapy or therapeutic agent is capecitabine.

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), and the additional therapy or therapeutic agent is sacituzumab govitecan (e.g., sacituzumab govitecan-hziy, or a biosimilar thereof).

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), and the additional therapy or therapeutic agent is trastuzumab deruxtecan (e.g., fam-trastuzumab deruxtecan-nxki, or a biosimilar thereof).

In some embodiments, the cancer is breast cancer (e.g., HER2+ breast cancer (e.g., HER2+ breast cancer with ER expression, HER2+ breast cancer without ER expression), HER2 negative breast cancer (e.g., HER2 negative breast cancer with ER expression, HER2 negative breast cancer without ER expression), triple negative breast cancer, or HER2 low breast cancer), and the additional therapy or therapeutic agent is trastuzumab (e.g., HERCEPTIN® (trastuzumab), or a biosimilar thereof (e.g., FACEPTOR® (trastuzumab), HERTICAD® (trastuzumab), TUZNUE® (trastuzumab), ZERCEPAC® (trastuzumab), trastuzumab-anns, trastuzumab-dkst, trastuzumab-dttb, trastuzumab-pkrb, trastuzumab-qyyp, EG-12014, or TX-05)).

In some embodiments, the cancer is hormone receptor positive breast cancer, and the additional therapy or therapeutic agent is hormone therapy (e.g., tamoxifen, toremifene, or a combination thereof).

In some embodiments, the cancer is hormone receptor positive breast cancer, and the additional therapy or therapeutic agent is a selective estrogen receptor degrader (SERD) (e.g., fulvestrant, elacestrant, or a combination thereof).

In some embodiments, the cancer is a HER2+ breast cancer (e.g., HER2+ breast cancer with ER expression, HER2+ breast cancer without ER expression), and the additional therapy or therapeutic agent is trastuzumab deruxtecan (e.g., fam-trastuzumab deruxtecan-nxki, or a biosimilar thereof) or trastuzumab emtansine (e.g., KADCYLA® (trastuzumab emtansine), or a biosimilar thereof (e.g., UJVIRA® (trastuzumab emtansine))).

In some embodiments, the cancer is a HER2 low breast cancer, and the additional therapy or therapeutic agent is trastuzumab deruxtecan (e.g., fam-trastuzumab deruxtecan-nxki, or a biosimilar thereof).

In some embodiments, the cancer is a head and neck cancer, and the additional therapy or therapeutic agent is a microtubule inhibitor (e.g., cabazitaxel, docetaxel, nab-paclitaxel, or paclitaxel). In some embodiments, the cancer is a head and neck cancer, and the additional therapy or therapeutic agent is docetaxel.

In some embodiments, the cancer is a cervical cancer, and the additional therapy or therapeutic agent is a microtubule inhibitor (e.g., cabazitaxel, docetaxel, nab-paclitaxel, or paclitaxel).

In some embodiments, the cancer is an endometrial cancer, and the additional therapy or therapeutic agent is a microtubule inhibitor (e.g., cabazitaxel, docetaxel, nab-paclitaxel, or paclitaxel).

In some embodiments, the cancer is a prostate cancer, and the additional therapy or therapeutic agent is a microtubule inhibitor (e.g., cabazitaxel, docetaxel, nab-paclitaxel, or paclitaxel).

In some embodiments, the cancer is an ovarian cancer (e.g., HGSOC) and the additional therapy or therapeutic agent is a microtubule inhibitor (e.g., cabazitaxel, docetaxel, nab-paclitaxel, or paclitaxel). In some embodiments, the cancer is an ovarian cancer (e.g., HGSOC) and the additional therapy or therapeutic agent is a microtubule inhibitor (e.g., cabazitaxel, docetaxel, nab-paclitaxel, or paclitaxel) and a platinum complex (e.g., carboplatin, cisplatin, lobaplatin, miriplatin, or oxaliplatin). In some embodiments, the cancer is an ovarian cancer (e.g., HGSOC) and the additional therapy or therapeutic agent is nab-paclitaxel or paclitaxel and carboplatin.

In some embodiments, the cancer is a pancreatic cancer, and the additional therapy or therapeutic agent is a microtubule inhibitor (e.g., cabazitaxel, docetaxel, nab-paclitaxel, or paclitaxel). In some embodiments, the cancer is a pancreatic cancer, and the additional therapy or therapeutic agent is a microtubule inhibitor (e.g., cabazitaxel, docetaxel, nab-paclitaxel, or paclitaxel) and gemcitabine.

In some embodiments, the cancer is a bladder cancer (e.g., bladder urothelial carcinoma), and the additional therapy or therapeutic agent is enfortumab vedotin (e.g., enfortumab vedotin-ejfv, or a biosimilar thereof).

In some embodiments, the cancer is a bladder cancer (e.g., bladder urothelial carcinoma), and the additional therapy or therapeutic agent is sacituzumab govitecan (e.g., sacituzumab govitecan-hziy, or a biosimilar thereof).

In some embodiments, the cancer is triple negative breast cancer, and the additional therapy or therapeutic agent is sacituzumab govitecan (e.g., sacituzumab govitecan-hziy, or a biosimilar thereof).

In some embodiments, folate receptor positivity status can be detected in a sample from the subject (e.g., via immunohistochemistry (IHC) and/or fluorescent in situ hybridization (FISH)).

In some embodiments, the subject was determined (e.g., prior to administration of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a))) to have a cancer that is folate receptor positive.

In some embodiments, the cancer is folate receptor positive ovarian cancer (e.g., folate receptor positive HGSOC), and the additional therapy or therapeutic agent is mirvetuximab soravtansine (e.g., mirvetuximab soravtansine-gynx, or a biosimilar thereof).

In some embodiments the immunomodulatory imide drug is thalidomide, lenalidomide, pomalidomide, iberdomide, avadomide, CC-99282, or a combination thereof.

In some embodiments, the anti-PD1 therapy is balstilimab, budigalimab, cadonilimab, camrelizumab, cemiplimab (e.g., cemiplimab-rwlc, or a biosimilar thereof), cetrelimab, dostarlimab (e.g., dostarlimab-gxly, or a biosimilar thereof), ezabenlimab, geptanolimab, ivonescimab, nivolumab (e.g., OPDIVO® (nivolumab), or a biosimilar thereof), nofazinlimab, pembrolizumab (e.g., KEYTRUDA® (pembrolizumab), or a biosimilar thereof), penpulimab, pidilizumab, pimivalimab, prolgolimab, pucotenlimab, retifanlimab (e.g., retifanlimab-dlwr, or a biosimilar thereof), rilvegostomig, rosnilimab, rulonilimab, sasanlimab, serplulimab, sintilimab (e.g., TYVYT® (sintilimab), or a biosimilar thereof), spartalizumab, tebotelimab, tislelizumab, toripalimab, volrustomig, vudalimab, zimberelimab, QL-1604, HX-009, INCB-086550, RG-6139, BAT-1306, SG-001, biosimilars thereof, or a combination thereof.

In some embodiments, the PD-L1 inhibitor is INCB-086550.

In some embodiments, the anti-PD-L1 therapy is adebrelimab, atezolizumab (e.g., TECENTRIQ® (atezolizumab), or a biosimilar thereof), avelumab (e.g., BAVENCIO® (avelumab), or a biosimilar thereof), bintrafusp alfa, cosibelimab, danburstotug, durvalumab (e.g., IMFINZI® (durvalumab), or a biosimilar thereof), envafolimab (e.g., ENWEIDA® (envafolimab), or a biosimilar thereof), erfonrilimab, pacmilimab, socazolimab, sugemalimab (e.g., CEJEMLY® (sugemalimab), or a biosimilar thereof), A-167, APL-502, AUPM-170, BNT-311, SHR-1701, biosimilars thereof, or a combination thereof.

In some embodiments, the additional therapy is radiotherapy.

In some embodiments, the cancer is head and neck cancer (e.g., head and neck squamous cell carcinoma), and the additional therapy is radiotherapy.

In some embodiments, the additional therapy includes a BRaf inhibitor and a MEK inhibitor. For example, the additional therapy can include dabrafenib and trametinib, vemurafenib and cobimetinib, or encorafenib and binimetinib.

Exemplary descriptions of agents in combination with BCL-2 family inhibitors can be found in: Hikita, Hayato, et al. Hepatology 52.4 (2010): 1310-1321; Chen, Jun, et al. Molecular Cancer Therapeutics 10.12 (2011): 2340-2349; Inuzuka, Hiroyuki, et al. Nature 471.7336 (2011): 104-109; Wertz, Ingrid E., et al. Nature 471.7336 (2011): 110-114; Tan, Nguyen, et al. Clinical Cancer Research 17.6 (2011): 1394-1404; Wong, Maureen, et al. Molecular Cancer Therapeutics 11.4 (2012): 1026-1035; Corcoran, Ryan B., et al. Cancer Cell 23.1 (2013): 121-128; Waibel, Michaela, et al. Cell Reports 5.4 (2013): 1047-1059; Frederick, Dennie T., et al. PloS One 9.7 (2014): e101286; Vlahovic, Gordana, et al. Investigational New Drugs 32.5 (2014): 976-984; Faber, Anthony C., et al. Cancer Discovery 4.1 (2014): 42-52; Leverson, Joel D., et al. Science Translational Medicine 7.279 (2015): 279ra40-279ra40; Guo, Jun, et al. PLoS One 10.3 (2015): e0114363; Lheureux, Stéphanie, et al. International Journal of Cancer 136.5 (2015): E340-E350; Zoeller, Jason J., et al. Cancer Research 76.14_Supplement (2016): 4358-4358; Weeden, Clare E., et al. Oncogene 37.32 (2018): 4475-4488; Lucantoni, Federico, et al. CellDeath & Disease 9.2 (2018): 1-13; Iavarone, Claudia, et al. Molecular Cancer Therapeutics 18.3 (2019): 642-655; Fleury, Hubert, et al. Nature Communications 10.1 (2019): 1-15; Lohard, Steven, et al. Nature Communications 11.1 (2020): 1-16; Bertino, Erin M., et al. Clinical Cancer Research 27.6 (2021): 1604-1611; Guo, Ting, et al. Aging 13.15 (2021): 19750; Puglisi, Martina, et al. Future Oncology 17.21 (2021): 2747-2758; Harrison, Claire N., et al. Journal of Clinical Oncology 40.15 (2022): 1671; Köhler, Jens, et al. Molecular Cancer Therapeutics 20.4 (2021): 641-654; Jaaks, Patricia, et al. Nature 603.7899 (2022): 166-173; Sobol, Benjamin, et al. International Journal of Molecular Sciences 23.14 (2022): 7850; Passamonti, J Clin Oncol 40, (2022) (suppl 16; abstr 7015); Qin, J Clin Oncol 40, (2022) (suppl 16; abstr e20612); Potter, Danielle S., et al. Cancer Research 82.12_Supplement (2022): 3691-3691; and Shebl, Bassem, et al. Cancer Research 82.12_Supplement (2022): 3888-3888.

Also provided herein is a method of treating cancer, comprising administering to a subject in need thereof (a) a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, and (b) an additional therapeutic agent, for simultaneous, separate or sequential use for the treatment of cancer, wherein the amounts of the compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, and the additional therapeutic agent are together effective in treating the cancer. In some embodiments, the method comprises administering (c) at least one pharmaceutically acceptable carrier.

These additional therapeutic agents may be administered with one or more doses of the compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, or pharmaceutical composition thereof, as part of the same or separate dosage forms, via the same or different routes of administration, and/or on the same or different administration schedules according to standard pharmaceutical practice known to one skilled in the art. In some embodiments, the compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, and the additional therapeutic agent are administered simultaneously as separate dosages. In some embodiments, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I—F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, and the additional therapeutic agent are administered as separate dosages sequentially in any order, in jointly therapeutically effective amounts, e.g., in daily or intermittently dosages. In some embodiments, the compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, and the additional therapeutic agent are administered simultaneously as a combined dosage.

Also provided herein is (i) a pharmaceutical combination for treating a cancer in a subject in need thereof, which comprises (a) a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, and (b) at least one additional therapeutic agent (e.g., any of the exemplary additional therapeutic agents described herein or known in the art), for simultaneous, separate or sequential use for the treatment of cancer, wherein the amounts of the compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, and of the additional therapeutic agent are together effective in treating the cancer; (ii) a pharmaceutical composition comprising such a combination; (iii) the use of such a combination for the preparation of a medicament for the treatment of cancer; and (iv) a commercial package or product comprising such a combination as a combined preparation for simultaneous, separate or sequential use; and to a method of treatment of cancer in a subject in need thereof. In some embodiments, the pharmaceutical combination comprises (c) at least one pharmaceutically acceptable carrier.

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, the terms “subject,” “individual,” or “patient,” are used interchangeably, refers to any animal, including mammals such as mice, rats, other rodents, rabbits, dogs, cats, swine, cattle, sheep, horses, primates, and humans. In some embodiments, the subject is a human. In some embodiments, the subject has experienced and/or exhibited at least one symptom of the disease, disorder, or condition to be treated and/or prevented.

In some embodiments, the subject is a pediatric subject.

The term “pediatric subject” as used herein refers to a subject under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. In some embodiments, a pediatric subject is from birth through the first 28 days of life, from 29 days of age to less than two years of age, from two years of age to less than 12 years of age, or 12 years of age through 21 years of age (up to, but not including, the twenty-second birthday). In some embodiments, a pediatric subject is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age.

The term “preventing” as used herein means to delay the onset, recurrence or spread, in whole or in part, of the disease or condition as described herein, or a symptom thereof.

The term “regulatory agency” refers to a country's agency for the approval of the medical use of pharmaceutical agents with the country. For example, a non-limiting example of a regulatory agency is the U.S. Food and Drug Administration (FDA).

The phrase “therapeutically effective amount” means an amount of compound that, when administered to a subject in need thereof, is sufficient to (i) treat a disease, disorder, or condition, (ii) attenuate, ameliorate, or eliminate one or more symptoms of the particular disease, disorder, or condition, or (iii) delay the onset of one or more symptoms of the particular disease, disorder, or condition described herein. The amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, that will correspond to such an amount will vary depending upon factors such as the particular compound, disease condition and its severity, the identity (e.g., weight) of the subject in need of treatment, but can nevertheless be routinely determined by one skilled in the art.

The phrase “effective amount” of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), as used herein, means an amount of the compound that, when administered to a cell, in vitro or in vivo, is sufficient to reduce proliferation of the cell or to kill the cell. The amount of a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, that will correspond to such an amount will vary depending upon factors such as the particular compound and genetics of the cell to be treated, but can nevertheless be routinely determined by one skilled in the art.

Pharmaceutical Compositions and Administration

General

In some embodiments, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, is administered as a pharmaceutical composition that includes the compound, or a pharmaceutically acceptable salt thereof, and one or more pharmaceutically acceptable excipients, and optionally one or more additional therapeutic agents as described herein.

In some embodiments, the compounds can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-a-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a compound as described herein in the range of 0.005% to 100% with the balance made up from non-toxic excipient may be prepared. The contemplated compositions may contain 0.001%-100% of a compound provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

Routes of Administration and Composition Components

In some embodiments, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, as described herein, or a pharmaceutical composition thereof, can be administered to a subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, intravitreal, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

In some embodiments, a compound of Formula (I) (e.g., (I-A) (e.g., (I-A-1), (I-A-2), or (I-A-3)), (I-B) (e.g., (I-B-1), (I-B-2), or (I-B-3)), (I-C), (I-D), (I-E) (e.g., (I-E-1), (I-E-2), or (I-E-3)), (I-Ea) (e.g., (I-Ea-1), (I-Ea-2), or (I-Ea-3)), (I-Eb) (e.g., (I-Eb-1), (I-Eb-2), or (I-Eb-3)), (I-F) (e.g., (I-F-1), (I-F-2), or (I-F-3)), (I-G), (I-Ga), (I-H) (e.g., (I-H-1), (I-H-2), or (I-H-3))) or Formula (II) (e.g., (II-a)), or a pharmaceutically acceptable salt thereof, as described herein, or a pharmaceutical composition thereof, can be administered orally to a subject in need thereof. Without being bound by any particular theory, it is believed that oral dosing (e.g., versus IV dosing) can be preferred by patients for convenience, perception of efficacy, and/or past experience.

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the compound described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the compounds described herein, or a pharmaceutical composition thereof, are suitable for local delivery to the digestive or GI tract by way of oral administration (e.g., solid or liquid dosage forms.).

Solid dosage forms for oral administration include capsules, tablets, pills, powders, and granules. In such solid dosage forms, the compound is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a compound, or pharmaceutically acceptable salt thereof, as provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives, or the like. In another solid dosage form, a powder, marume, solution or suspension (e.g., in propylene carbonate, vegetable oils, PEGs, poloxamer 124 or triglycerides) is encapsulated in a capsule (gelatin or cellulose base capsule). Unit dosage forms in which one or more compounds provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the compounds to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier, and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic, or amphoteric surfactant. As with other carriers or vehicles, an ointment base should be inert, stable, nonirritating, and non-sensitizing.

In any of the foregoing embodiments, pharmaceutical compositions described herein can include one or more one or more of the following: lipids, interbilayer crosslinked multilamellar vesicles, biodegradable poly(D,L-lactic-co-glycolic acid) [PLGA]-based or poly anhydride-based nanoparticles or microparticles, and nanoporous particle-supported lipid bilayers.

Dosages

The dosages may be varied depending on the requirement of the patient, the severity of the condition being treated, and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 1 mg/kg; from about 0.1 mg/kg to about 0.5 mg/kg).

Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

The term “acceptable” with respect to a formulation, composition, or ingredient, as used herein, means having no persistent detrimental effect on the general health of the subject being treated.

“API” refers to an active pharmaceutical ingredient.

The term “excipient” or “pharmaceutically acceptable excipient” means a pharmaceutically acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, P A, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, F L, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid, and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The term “pharmacologically acceptable salts” is not specifically limited as far as it can be used in medicaments. Examples of a salt that the compounds described herein form with a base include the following: salts thereof with inorganic bases such as sodium, potassium, magnesium, calcium, and aluminum; salts thereof with organic bases such as methylamine, ethylamine, and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to a subject. Multiple techniques of administering a compound exist in the art including, but not limited to: rectal, oral, intravenous, aerosol, parenteral, ophthalmic, pulmonary, and topical administration.

Compound Preparation

The compounds disclosed herein can be prepared in a variety of ways using commercially available starting materials, compounds known in the literature, or from readily prepared intermediates, by employing standard synthetic methods and procedures either known to those skilled in the art, or in light of the teachings herein.

Standard synthetic methods and procedures for the preparation of organic molecules and functional group transformations and manipulations can be obtained from the relevant scientific literature or from standard textbooks in the field. Although not limited to any one or several sources, classic texts such as R. Larock, Comprehensive Organic Transformations, VCH Publishers (1989); L. Fieser and M. Fieser, Fieser and Fieser's Reagents for Organic Synthesis, John Wiley and Sons (1994); Smith, M. B., March, J., March's Advanced Organic Chemistry: Reactions, Mechanisms, and Structure, 5th edition, John Wiley & Sons: New York, 2001; and Greene, T. W., Wuts, P. G. M., Protective Groups in Organic Synthesis, 3rd edition, John Wiley & Sons: New York, 1999, are useful and recognized reference textbooks of organic synthesis known to those in the art. The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt thereof.

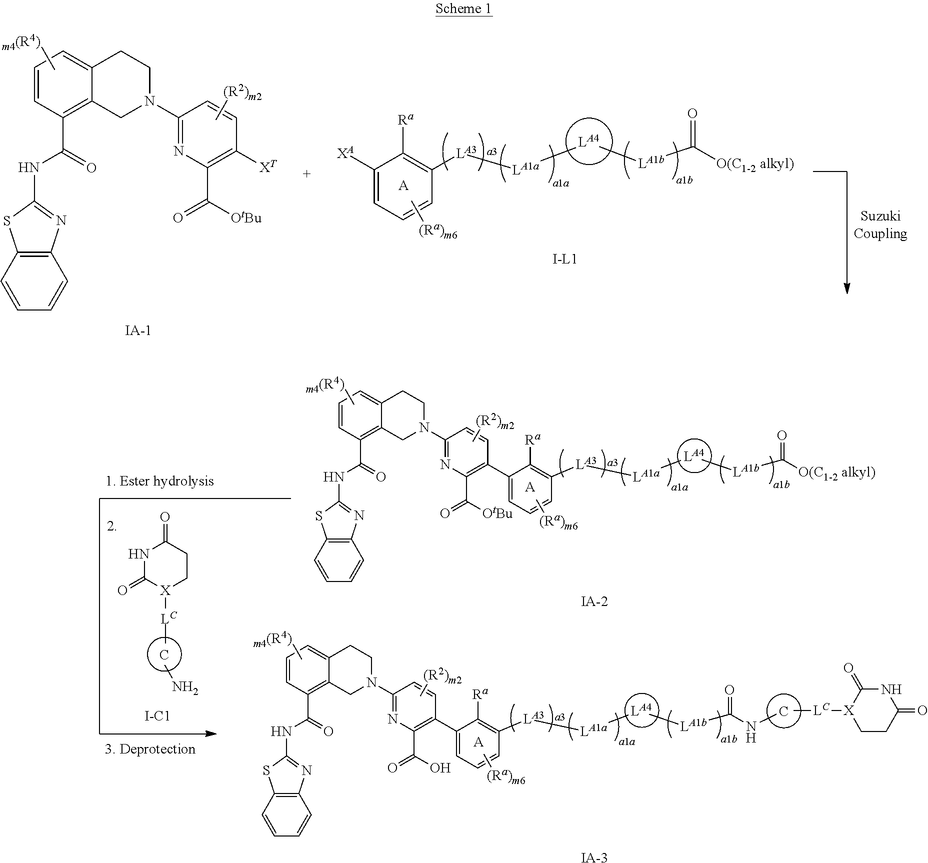

Scheme 1 depicts the synthesis of Compound IA-3 which is a compound of Formula (I-A), wherein R¹ is C(O)OH; and each remaining variable is defined in accordance with Formula (I-A). Compound IA-1 is reacted with Compound I-L1 under standard conditions for Suzuki coupling to provide Compound IA-2, wherein one of X^{A} and X^{T} is B(OH)₂ or Bpin; the other of X^{A} and X^{T} is —Br; and each remaining variable in IA-1 and I-L1 is defined in accordance with Formula (I-A). Hydrolysis of the C₁₋₂ alkyl ester in IA-2 provides the corresponding carboxylic acid which is then reacted with I-C1 under standard conditions for amide bond formation, wherein each variable in I-C1 is defined in accordance with Formula (I-A). Removal of the tert-butyl group under standard conditions for deprotection then provides Compound IA-3.

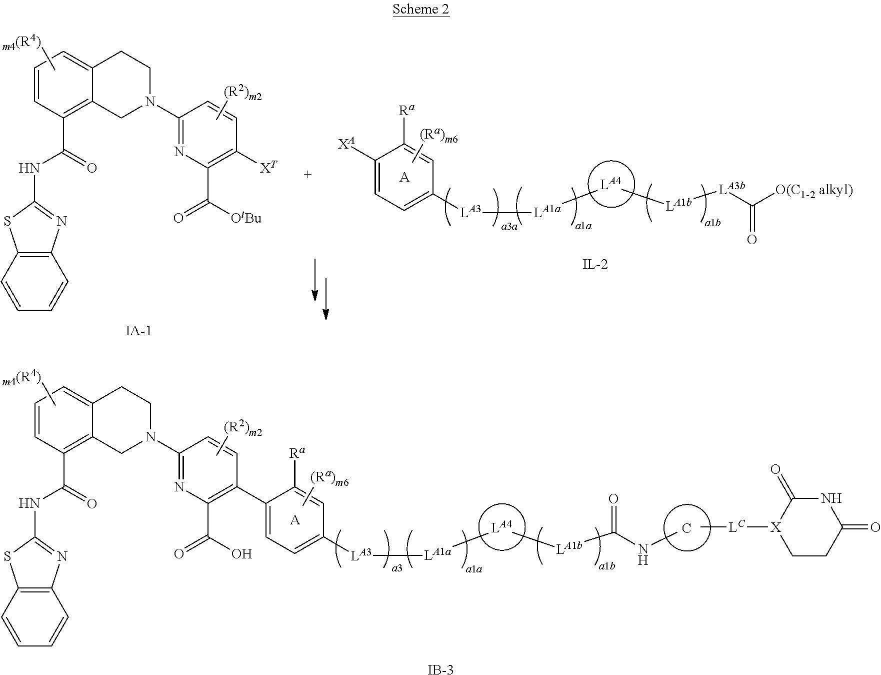

Compound IB-3 which is a compound of Formula (I-B), wherein R¹ is C(O)OH; and each remaining variable is defined in accordance with Formula (I-B), is prepared from IA-1 and I-L2 using methods depicted for Compound IA-3 in Scheme 1, above. One of X^{A} and X^{T} is B(OH)₂ or Bpin; the other of X^{A} and X^{T} is —Br; and each remaining variable in IA-1 and I-L2 is defined in accordance with Formula (I-B).

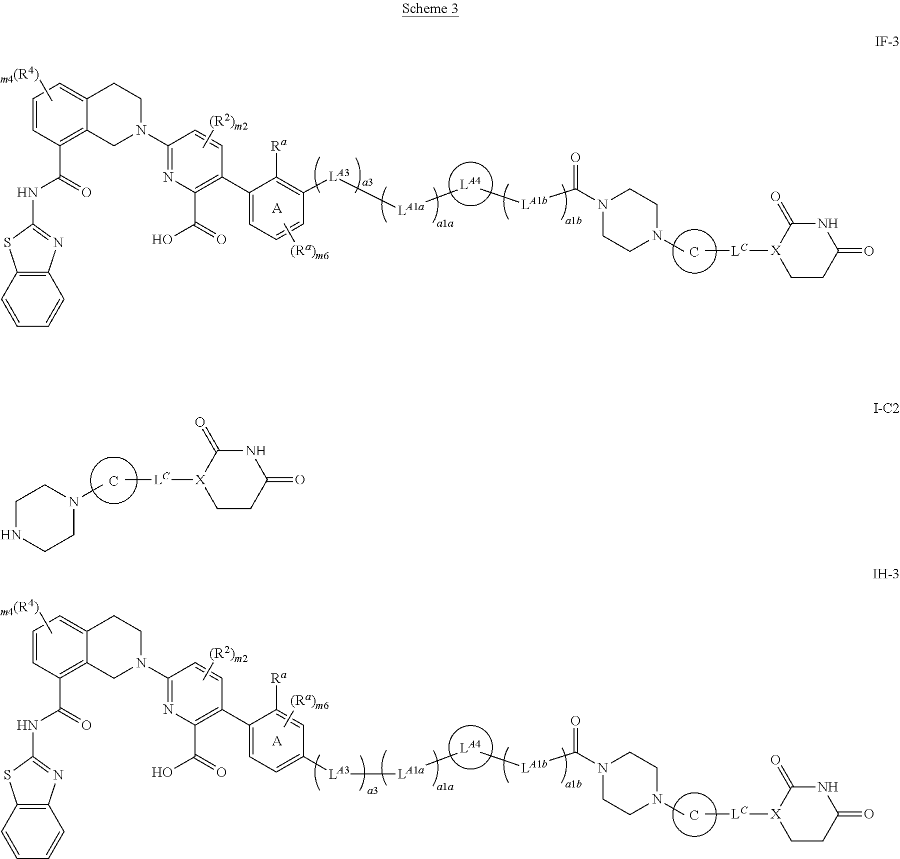

Analogously, as shown in Scheme 3, Compound IF-3 (a compound of Formula (I-F), wherein R¹ is C(O)OH; and each remaining variable is defined in accordance with Formula (I-F)) and Compound IH-3 (a compound of Formula (I-H), wherein R¹ is C(O)OH; and each remaining variable is defined in accordance with Formula (I-H)) are prepared with methods similar to those depicted in Schemes 1 and 2 by using I-C2 (wherein each variable is defined in accordance with Formula (I-F) or (I-H)) instead of I-C1.

Also provided herein are compounds of Formula (SI-A) and Formula (SI-B):

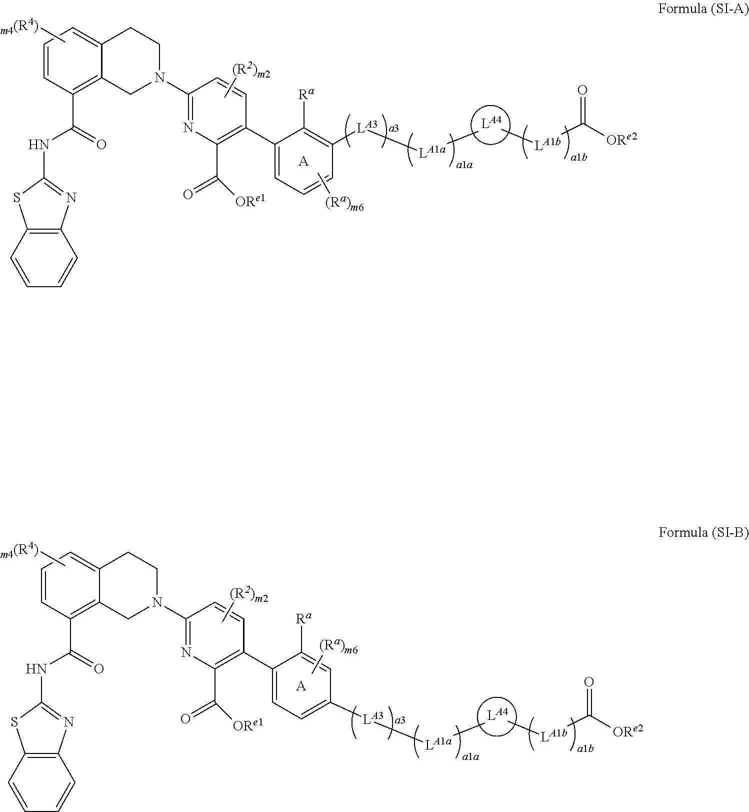

or salts thereof, wherein:
each R² and R⁴ is independently selected from: halo, CN, C₁₋₃ alkyl, C₁₋₃ haloalkyl, C₁₋₃ alkoxy, C₁₋₃ haloalkoxy, OH, and NR^{d}R^{e};
m2 is 0, 1, or 2;
m4 is 0, 1, 2, or 3;
m6 is 0 or 1;
R^{e1} is H or C₁₋₆ alkyl optionally substituted with 1-3-F;
R^{e2} is H or C₁₋₆ alkyl optionally substituted with 1-3-F;
a3 is 0 or 1;
L^{A3} is selected from the group consisting of: —O—, —N(H)—, and —N(C₁₋₃ alkyl)-;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
a1a and a1b are independently integers from 0 to 5, provided that a1a+a1b is from 1 to 5;
each R^{L} is independently selected from the group consisting of: halo; cyano; —OH; —C₁₋₆ alkoxy; —C₁₋₆ haloalkoxy; —NR^{d}R^{e}; C(═O)N(R^{f})₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{c};
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{e};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—; —N(H)—; —N(C₁₋₃ alkyl)-; —S(O)₀₋₂—; C(═O); and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{e} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(R^{f})₂; S(O)₁₋₂(C₁₋₆ alkyl); S(O)₁₋₂(C₁₋₆ haloalkyl); S(O)₁₋₂N(R^{f})₂; and C₁₋₆ alkyl optionally substituted with 1-3 R^{h}.
each R^{f} is independently selected from the group consisting of: H and C₁₋₆ alkyl optionally substituted with 1-3 R^{h}; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂.

In some embodiments of Formula (SI-A) and Formula (SI-B), m2 is 0; m4 is 0; and m6 is 0.

In some embodiments of Formula (SI-A) and Formula (SI-B), each R^{a} is independently C₁₋₆ alkyl optionally substituted with 1-3-F (e.g., CH₃ or CF₃).

In some embodiments of Formula (SI-A) and Formula (SI-B), m2 is 0; m4 is 0; and m6 is 0; and R^{a} is C₁₋₆ alkyl optionally substituted with 1-3-F (e.g., CH₃ or CF₃).

In some embodiments of Formula (SI-A) and Formula (SI-B), a3 is 1. In some embodiments of Formula (SI-A) and Formula (SI-B), L^{A3} is —O—. In some embodiments of Formula (SI-A) and Formula (SI-B), a3 is 1; and L^{A3} is —O—.

In some embodiments of Formula (SI-A) and Formula (SI-B), a1a+a1b is 3 or 4. For example, a1a+a1b can be 3. For example, a1a+a1b can be 4.

In some embodiments of Formula (SI-A) and Formula (SI-B), a1a+a1b is 2 or 5. For example, a1a+a1b can be 5.

In some embodiments of Formula (SI-A) and Formula (SI-B), each occurrence of L^{A1a} and L^{A1b} is —CH₂—. In some embodiments of Formula (SI-A) and Formula (SI-B), one occurrence of L^{A1a} is —CHR^{L}— or —C(R^{L})₂—; each remaining occurrence of L^{A1a} is —CH₂—; and each occurrence of L^{A1b} is —CH₂—. In some embodiments of Formula (SI-A) and Formula (SI-B), one occurrence of L^{A1b} is —CHR^{L}— or —C(R^{L})₂—; each remaining occurrence of L^{A1b} is —CH₂—; and each occurrence of L^{A1a} is —CH₂—. In some embodiments of Formula (SI-A) and Formula (SI-B), each R^{L} is independently selected from the group consisting of: —F and —C₁₋₃ alkyl optionally substituted with 1-3 F (e.g., CH₃ or CF₃).

In some embodiments, the compounds of Formula (SI-A) and Formula (SI-B) are compounds of Formula (SI-A-1) and (SI-B-1), respectively:

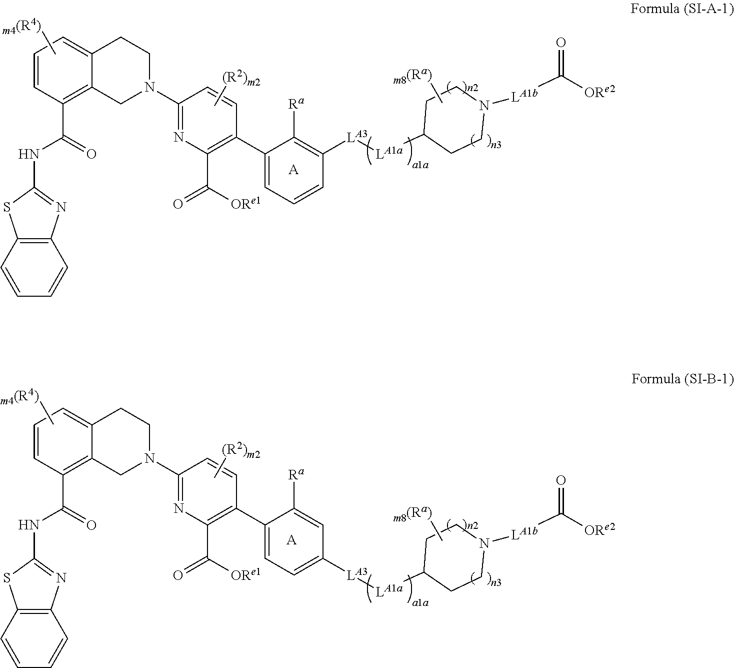

or salts thereof, wherein:
each R² and R⁴ is independently selected from: halo, C₁₋₃ alkyl, C₁₋₃ haloalkyl, C₁₋₃ alkoxy, and C₁₋₃ haloalkoxy;
m2 is 0 or 1;
m4 is 0 or 1;
a1a is 0, 1, 2, or 3;
n2 is 0 or 1;
n3 is 0 or 1;
m8 is 0, 1, or 2;
each R^{a} is independently selected from the group consisting of: —F and —C₁₋₃ alkyl optionally substituted with 1-3 F (e.g., CH₃ or CF₃);
R^{e1} is H or C₁₋₆ alkyl optionally substituted with 1-3-F;
R^{e2} is H or C₁₋₆ alkyl optionally substituted with 1-3-F;
L^{A3} is selected from the group consisting of: —O— and —N(H)—;
L^{A1a} and L^{A1b} are independently selected from the group consisting of: —CH₂—, —CHR^{L}—, and —C(R^{L})₂—;
a1a is an integer from 0 to 3; and
each R^{L} is independently selected from the group consisting of: —F and —C₁₋₃ alkyl optionally substituted with 1-3 F (e.g., CH₃ or CF₃).

In some embodiments of Formula (SI-A-1) and (SI-B-1), m2 is 0; m4 is 0; 0 to 1 occurrence of L^{A1a} is —CHR^{L}— or —C(R^{L})₂—; each remaining occurrence of L^{A1a} is —CH₂—; L^{A3} is —O—; and L^{A1b} is —CH₂—. For example, compounds of Formulas (SI-A-1) and (SI-B-1) can be:

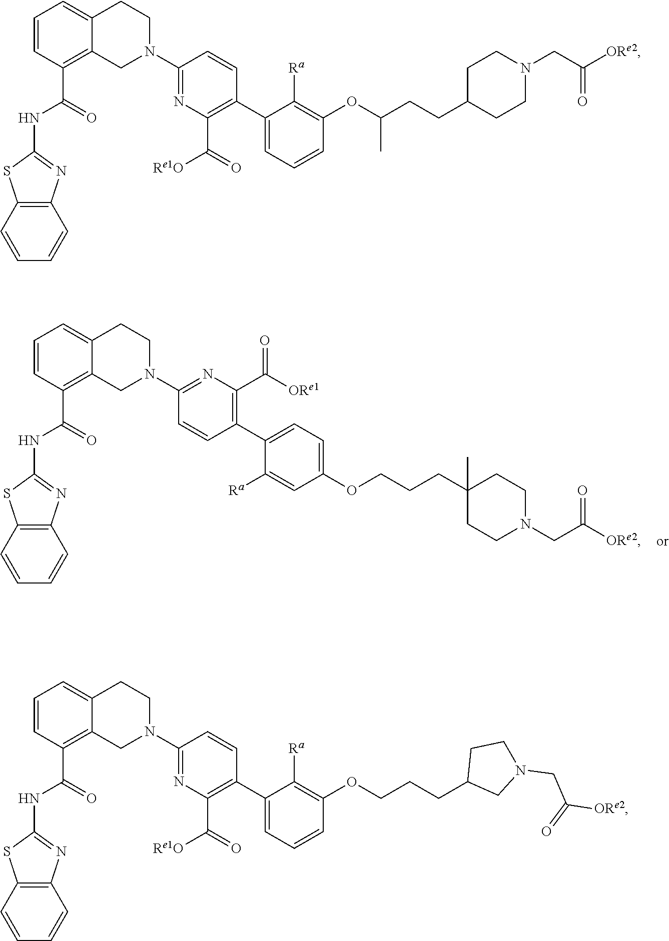

or salts thereof.

In some embodiments, the compounds of Formula (SI-A) and Formula (SI-B) are compounds of Formula (SI-A-2) or Formula (SI-B-2), respectively:

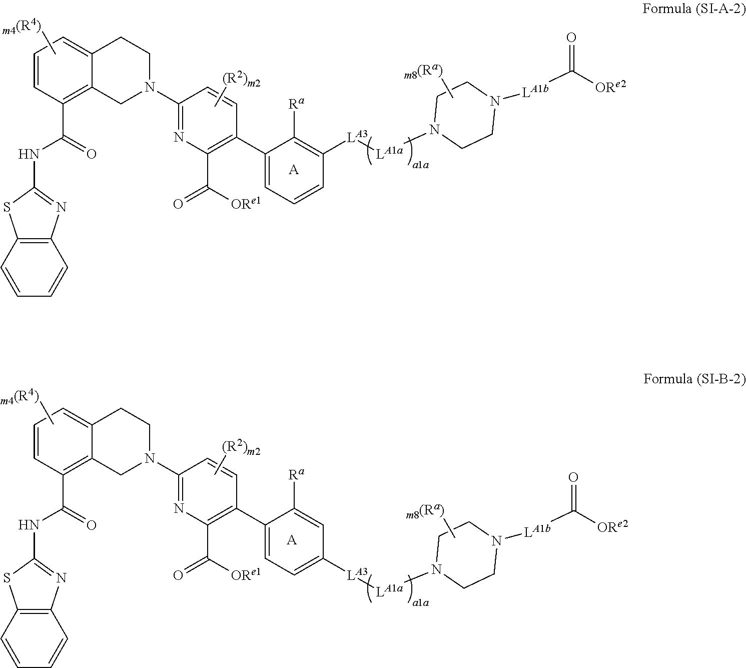

or salts thereof, wherein:
each R² and R⁴ is independently selected from: halo, C₁₋₃ alkyl, C₁₋₃ haloalkyl, C₁₋₃ alkoxy, and C₁₋₃ haloalkoxy;
m2 is 0 or 1;
m4 is 0 or 1;
a1a is 0, 1, 2, or 3;
m8 is 0, 1, or 2;
each R^{a} is independently selected from the group consisting of: —F and —C₁₋₃ alkyl optionally substituted with 1-3 F (e.g., CH₃ or CF₃);
R^{e1} is H or C₁₋₆ alkyl optionally substituted with 1-3-F;
R^{e2} is H or C₁₋₆ alkyl optionally substituted with 1-3-F;
L^{A3} is selected from the group consisting of: —O— and —N(H)—;
L^{A1a} and L^{A1b} are independently selected from the group consisting of: —CH₂—, —CHR^{L}—, and —C(R^{L})₂—;
a1a is an integer from 0 to 3; and
each R^{L} is independently selected from the group consisting of: —F and —C₁₋₃ alkyl optionally substituted with 1-3 F (e.g., CH₃ or CF₃).

In some embodiments of Formula (SI-A-2) and Formula (SI-B-2), m2 is 0; m4 is 0; 0 to 1 occurrence of L^{A1a} is —CHR^{L}— or —C(R^{L})₂—; each remaining occurrence of L^{A1a} is —CH₂—; L^{A3} is —O—; and L^{A1b} is —CH₂—. For example, compounds of Formula (SI-A-2) and Formula (SI-B-2) can be:

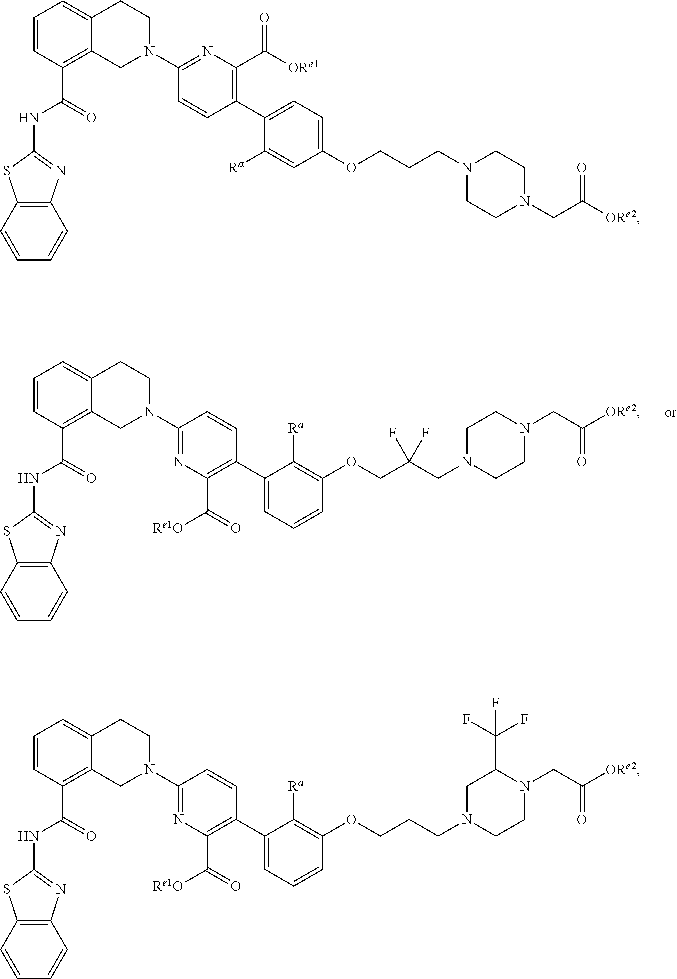

or salts thereof.

In some embodiments, the compounds of Formula (SI-A) or Formula (SI-B) are compounds of Formula (SI-A-3) or Formula (SI-B-3), respectively:

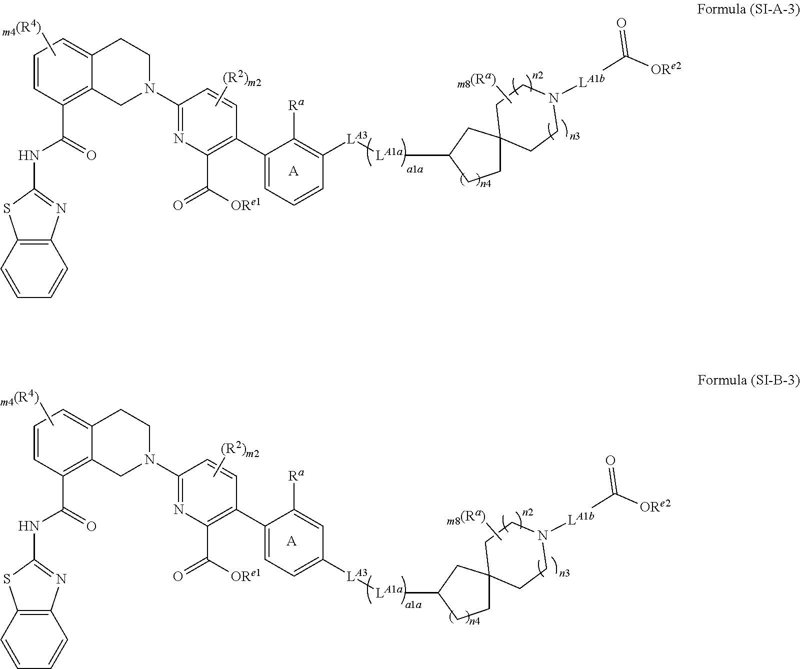

or salts thereof, wherein:
each R² and R⁴ is independently selected from: halo, C₁₋₃ alkyl, C₁₋₃ haloalkyl, C₁₋₃ alkoxy, and C₁₋₃ haloalkoxy;
m2 is 0 or 1;
m4 is 0 or 1;
a1a is 0, 1, 2, or 3;
n2 is 0 or 1;
n3 is 0 or 1;
n4 is 0, 1, or 2;
m8 is 0, 1, or 2;
R^{a} is independently selected from the group consisting of: —F and —C₁₋₃ alkyl optionally substituted with 1-3 F (e.g., CH₃ or CF₃);
R^{e1} is H or C₁₋₆ alkyl optionally substituted with 1-3-F;
R^{e2} is H or C₁₋₆ alkyl optionally substituted with 1-3-F;
L^{A3} is selected from the group consisting of: —O— and —N(H)—;
L^{A1a} and L^{A1b} are independently selected from the group consisting of: —CH₂—, —CHR^{L}—, and —C(R^{L})₂—;
a1a is an integer from 0 to 3; and
each R^{L} is independently selected from the group consisting of: —F and —C₁₋₃ alkyl optionally substituted with 1-3 F (e.g., CH₃ or CF₃).

In some embodiments of Formula (SI-A-3) and Formula (SI-B-3), m2 is 0; m4 is 0; m8 is 0; 0 to 2 occurrences of L^{A1a} is —CH₂—; L^{A3} is —O—; and L^{A1b} is —CH₂—. For example, compounds of Formula (SI-A-3) and Formula (SI-B-3) can be:

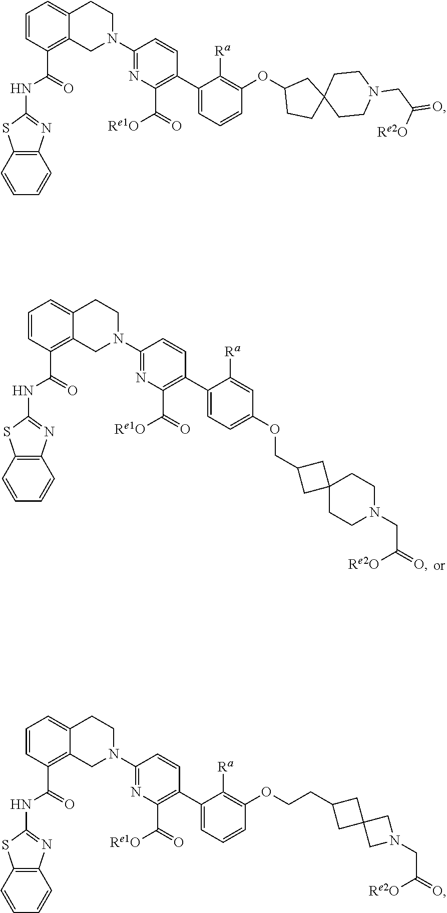

or salts thereof.

Exemplary embodiments of compounds of one or more of Formulas (SI-A), (SI-B), (SI-A-1), (SI-B-1), (SI-A-2), (SI-B-2), (SI-A-3), and (SI-B-3) include certain compounds described in Examples 1, 3, 4, 6, 7, 9, 10, 11, 12, 13, 14, 15, 17, 18, 19, 20, 21, 22, 25, 26, 27, 31, 32, 33, 35, 36, 38, 39, 40, 41, 42, 43, 45, 46, 47, 48, 50, 52, 54, 55, 56, 57, 60, 62, 63, 64, 67, 68, 69, 70, 71, 72, 77, 78, 80, 81, 84, 85, 86, 88, 89, 91, 92, 93, 94, 96, 97, 99, 100, 102, 103, 106, 107, 109, 110, 116, 117, 118, 119, 120, 121, 122, 131, 132, 133, 134, 135, 136, 138, 140, 141, 142, 143, 144, 147, 148, 149, 150, 151, 152, 153, 160, 161, 163, 168, 174, 175, 177, 181, 183, 187, 189, 190, 192, 193, 194, 199, 200, 201, 202, 203, 204, 205, 206, 215, 216, 223, 224, and 229.

Also provided herein are compounds of Formula (SI-E) and Formula (SI-G):

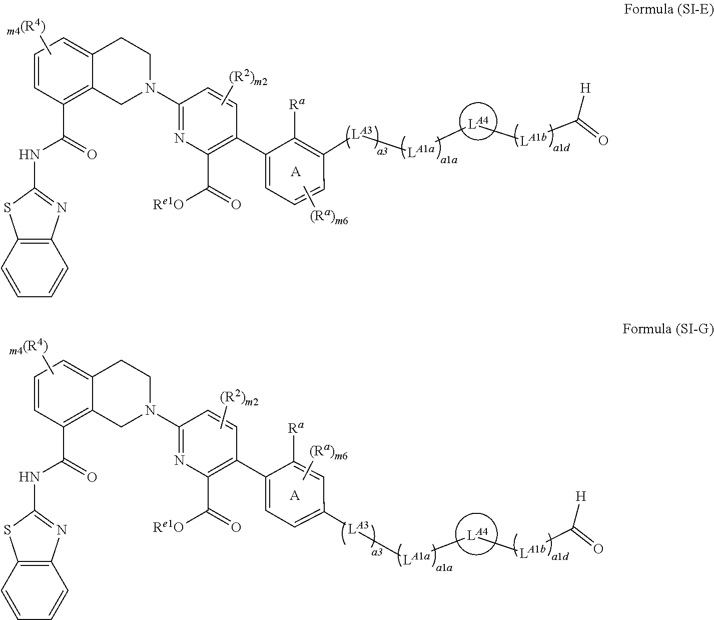

or salts thereof, wherein:
each R² and R⁴ is independently selected from: H, C₁₋₃ alkyl, and halo;
a3 is 0 or 1;
m2 is 0, 1, or 2;
m4 is 0, 1, 2, or 3;
m6 is 0 or 1;
R^{e1} is H or C₁₋₆ alkyl optionally substituted with 1-3-F;
L^{A3} is selected from the group consisting of: —O—, —N(H)—, and —N(C₁₋₃ alkyl)-;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
a1a is an integer from 0 to 5;
a1d is an integer from 0 to 4, provided that a1a+a1d is from 1 to 4;
each R^{L} is independently selected from the group consisting of: halo; cyano; —OH; —C₁₋₆ alkoxy; —C₁₋₆ haloalkoxy; —NR^{d}R^{e}; C(═O)N(R^{f})₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{c};
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—; —N(H)—; —N(C₁₋₃ alkyl)-; —S(O)₀₋₂—; C(═O); and C₁₋₃ alkylene;
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(Rr)₂; S(O)₁₋₂(C₁₋₆ alkyl); S(O)₁₋₂(C₁₋₆ haloalkyl); S(O)₁₋₂N(R^{f})₂; and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂.

In some embodiments of Formula (SI-E) and Formula (SI-G), m2 is 0; m4 is 0; and m6 is 0.

In some embodiments of Formula (SI-E) and Formula (SI-G), R^{a} is C₁₋₆ alkyl optionally substituted with 1-3-F (e.g., CH₃ or CF₃).

In some embodiments of Formula (SI-E) and Formula (SI-G), m2 is 0; m4 is 0; and m6 is 0; and R^{a} is C₁₋₆ alkyl optionally substituted with 1-3-F (e.g., CH₃ or CF₃).

In some embodiments of Formula (SI-E) and Formula (SI-G), a3 is 1. In some embodiments, L^{A3} is —O—. In some embodiments of Formula (SI-E) and Formula (SI-G), a3 is 1; and L^{A3} is —O—.

In some embodiments of Formula (SI-E) and Formula (SI-G), a1a+a1d is 2 or 5. For example, a1a+a1b can be 5. In some such embodiments each occurrence of L^{A1a} and L^{A1b} is —CH₂—.

In some embodiments of Formula (SI-E) and Formula (SI-G), a1a is 0; and a1d is 1, 2 or 3.

In some embodiments of Formula (SI-E) and Formula (SI-G), a1a is 0; and each occurrence of L^{A1b} is —CH₂—. In some embodiments of Formula (SI-E) and Formula (SI-G), a1a is 0; one occurrence of L^{A1b} is —CHR^{L}— or —C(R^{L})₂—; and each remaining occurrence of L^{A1b} is —CH₂—. In some embodiments of Formula (SI-E) and Formula (SI-G), each R^{L} is independently selected from the group consisting of: —F and —C₁₋₃ alkyl optionally substituted with 1-3 F (e.g., CH₃ or CF₃).

In some embodiments, the compounds of Formula (SI-E) are compounds of Formula (SI-E-1):

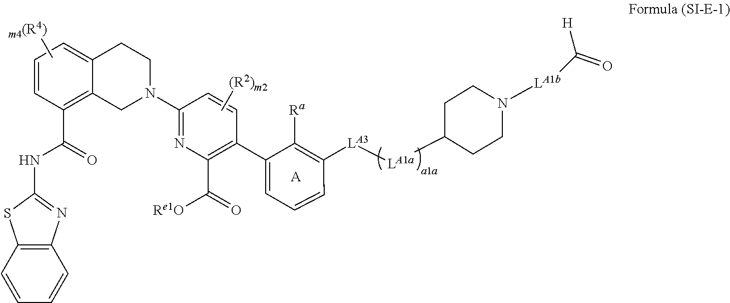

or salts thereof, wherein:
each R² and R⁴ is independently selected from: halo, C₁₋₃ alkyl, C₁₋₃ haloalkyl, C₁₋₃ alkoxy, and C₁₋₃ haloalkoxy;
m2 is 0 or 1;
m4 is 0 or 1;
a1a is 1, 2, or 3;
R^{a} is independently selected from the group consisting of: —F and —C₁₋₃ alkyl optionally substituted with 1-3 F (e.g., CH₃ or CF₃);
R^{e1} is H or C₁₋₆ alkyl optionally substituted with 1-3-F;
L^{A3} is selected from the group consisting of: —O— and —N(H)—;
L^{A1a} and L^{A1b} are independently selected from the group consisting of: —CH₂—, —CHR^{L}—, and —C(R^{L})₂—;
a1a is an integer from 0 to 3; and
each R^{L} is independently selected from the group consisting of: —F and —C₁₋₃ alkyl optionally substituted with 1-3 F (e.g., CH₃ or CF₃).

In some embodiments, of Formula (SI-E-1) m2 is 0; m4 is 0; 1-3 occurrences of L^{A1}a are —CH₂—; L^{A3} is —O—; and L^{A1b} is —CH₂—. For example, compounds of Formula (SI-E-1): can be:

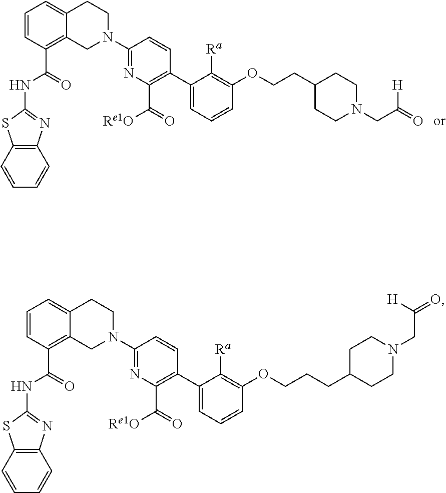

or salts thereof.

In some embodiments, the compounds of Formula (SI-E) and Formula (SI-G) are compounds of Formula (SI-E-2) and Formula (SI-G-2), respectively:

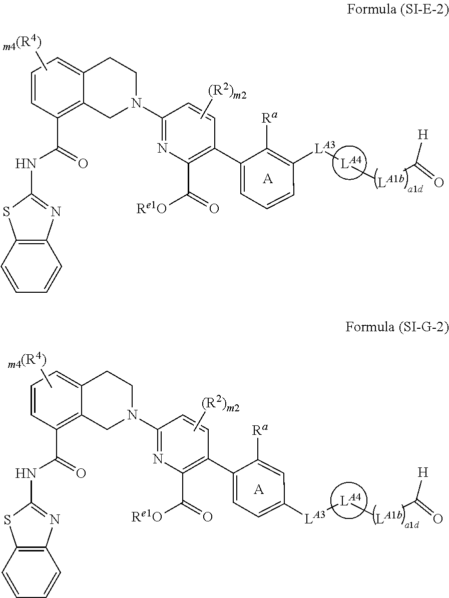

or salts thereof, wherein:
each R² and R⁴ is independently selected from: halo, C₁₋₃ alkyl, C₁₋₃ haloalkyl, C₁₋₃ alkoxy, and C₁₋₃ haloalkoxy;
m2 is 0 or 1;
m4 is 0 or 1;
a1d is 1, 2, or 3;
L^{A4} is cyclohexylene or phenylene (e.g., 1,4-cyclohexylene or 1,4-phenylene);
R^{a} is independently selected from the group consisting of: —F and —C₁₋₃ alkyl optionally substituted with 1-3 F (e.g., CH₃ or CF₃);
R^{e1} is H or C₁₋₆ alkyl optionally substituted with 1-3-F;
L^{A3} is selected from the group consisting of: —O— and —N(H)—;
L^{A1b} is independently selected from the group consisting of: —CH₂—, —CHR^{L}—, and —C(R^{L})₂—; and
each R^{L} is independently selected from the group consisting of: —F and —C₁₋₃ alkyl optionally substituted with 1-3 F (e.g., CH₃ or CF₃).

In some embodiments, of Formula (SI-E-2) and (SI-G-2) m2 is 0; m4 is 0; L^{A3} is —O—; and each occurrence of L^{A1b} is —CH₂—. For example, compounds of Formula (SI-E-2) and Formula (SI-G-2) can be:

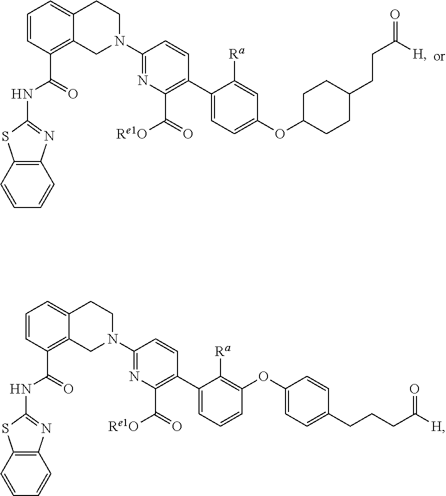

or salts thereof.

Exemplary embodiments of compounds of one or more of Formulas (SI-E), (SI-G), (SI-E-1), (SI-E-2), and (SI-G-2) include certain compounds described in Examples 8, 16, 28, 29, 34, 44, 53, 65, 66, 73, 74, 75, 76, 87, 95, 98, 111, 137, 154, 156, 158, 162, 164, 167, 169, 170, 171, 178, 182, 191, 195, 196, 197, 198, 212, 213, and 214.

Also provided herein are compounds of Formulas (I-Ind-1), (I-Ind-2), and (I-Ind-3):

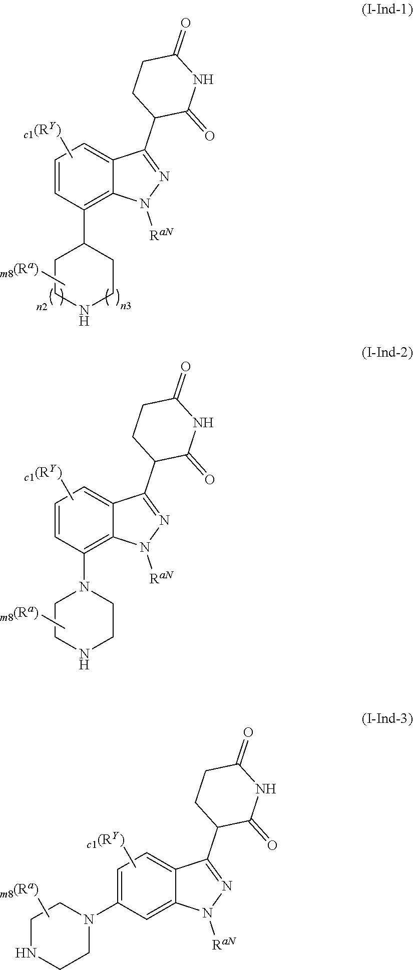

or salts thereof, wherein:
each R^{Y} is independently selected from the group consisting of: R^{a} and R^{b};
c1 is 0, 1, 2, or 3;
n2 and n3 are independently 0, 1, or 2;
R^{aN} is H or C₁₋₆ alkyl optionally substituted with 1-3 R^{e};
m8 is 0 to 4;
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—; —N(H)—; —N(C₁₋₃ alkyl)-; —S(O)₀₋₂—; C(═O); and C₁₋₃ alkylene;
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(Rr)₂; S(O)₁₋₂(C₁₋₆ alkyl); S(O)₁₋₂(C₁₋₆ haloalkyl); S(O)₁₋₂N(R^{f})₂; and C₁₋₆ alkyl optionally substituted with 1-3 R^{h}.
each R^{f} is independently selected from the group consisting of: H and C₁₋₆ alkyl optionally substituted with 1-3 R^{h};
each R^{g} is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂.

In some embodiments of Formulas (I-Ind-1), (I-Ind-2), and (I-Ind-3), c1 is 0.

In some embodiments of Formula (I-Ind-1), n2 and n3 are independently 1.

In some embodiments of Formulas (I-Ind-1), (I-Ind-2), and (I-Ind-3), R^{aN} is C₁₋₆ alkyl optionally substituted with 1-3 R^{e}.

In some embodiments of Formulas (I-Ind-1), (I-Ind-2), and (I-Ind-3), c1 is 0; and R^{aN} is C₁₋₆ alkyl optionally substituted with 1-3 R^{e}.

In some embodiments of Formula (I-Ind-2) and Formula (I-Ind-3), c1 is 0; R^{aN} is —CH₃; and R^{a} is selected from the group consisting of: —F and —C₁₋₃ alkyl optionally substituted with 1-3 F (e.g., CH₃ or CF₃). For example, compounds of Formula (I-Ind-2) and Formula (I-Ind-3) can be:

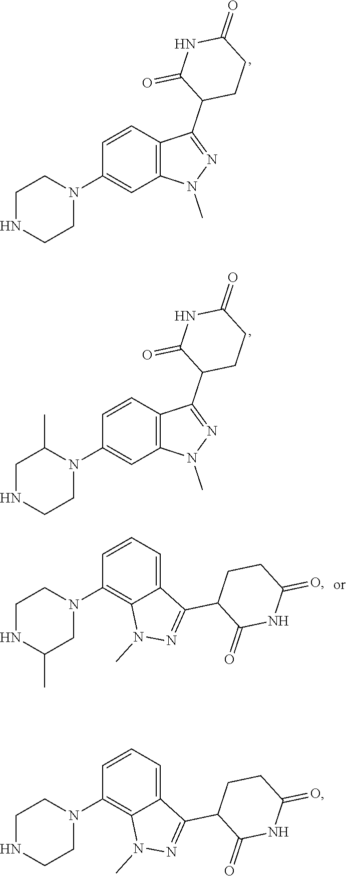 [SMILES: Cn1nc(C2CCC(=O)NC2=O)c2ccc(N3CCNCC3)cc21 ; CC1CNCCN1c1ccc2c(C3CCC(=O)NC3=O)nn(C)c2c1 ; CC1CN(c2cccc3c(C4CCC(=O)NC4=O)nn(C)c23)CCN1 ; Cn1nc(C2CCC(=O)NC2=O)c2cccc(N3CCNCC3)c21]

or salts thereof.

Exemplary embodiments of compounds of one or more of Formulas (I-Ind-1), (I-Ind-2), and (I-Ind-3) include certain compounds described in Examples 6, 7, 8, 9, 10, 11, 12, 13, 14, 15, 16, 25, 26, 27, 28, 29, 34, 38, 44, 49, 53, 58, 59, 61, 66, 67, 73, 74, 75, 76, 85, 86, 87, 90, 95, 96, 98, 101, 113, 137, 145, 147, 149, 150, 151, 152, 154, 156, 157, 158, 162, 163, 164, 167, 168, 169, 170, 171, 172, 175, 177, 178, 182, 184, 185, 191, 195, 196, 197, 207, 208, 209, 210, 211, 212, 213, and 214.

Also provided herein are compounds of Formula (SI-J) and Formula (SI-K):

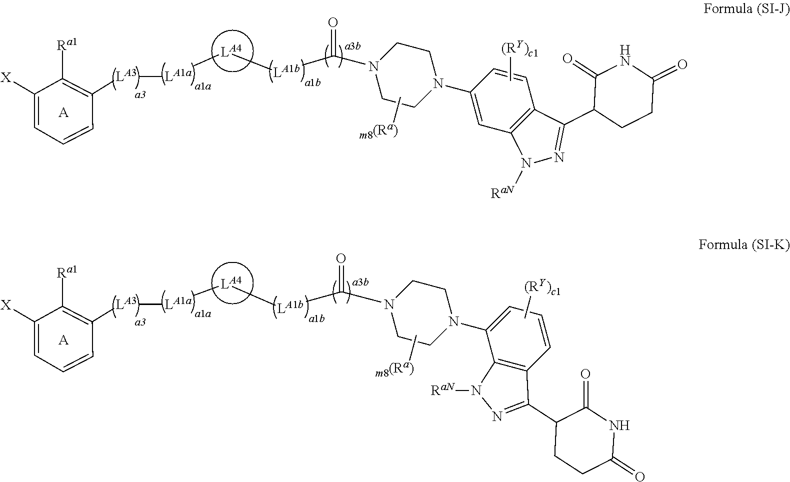

or salts thereof, wherein:
X is selected from the group consisting of:
halo;
—B(OH)₂;
—BF₃⁻;
B(OR^{T})₂, wherein each R^{T} is independently C₁₋₃ alkyl optionally substituted with 1-3 R^{c}; and

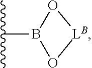

wherein L^{B} is C₂₋₆ alkylene optionally substituted with 1-4 R^{a};
R^{a1} is H or C₁₋₃ alkyl optionally substituted with 1-3 F;
L^{A3} is selected from the group consisting of: —O—, —N(H)—, and —N(C₁₋₃ alkyl)-;
a3 is 0 or 1;
a3b is 0 or 1;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a};
b) phenylene or 5-6 membered heteroarylene, each of which is optionally substituted with 1-3 R^{a};
a1a is an integer from 0 to 5;
a1b is an integer from 0 to 4, provided that a1a+a1b is from 1 to 4;
each R^{L} is independently selected from the group consisting of: halo; cyano; —OH; —C₁₋₆ alkoxy; —C₁₋₆ haloalkoxy; —NR^{d}R^{e}; C(═O)N(R^{f})₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{c};
each R^{Y} is independently selected from the group consisting of: R^{a} and R^{b};
c1 is 0, 1, 2, or 3;
R^{a}N is H or C₁₋₆ alkyl optionally substituted with 1-3 R^{c},
m8 is 0, 1, or 2.
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—; —N(H)—; —N(C₁₋₃ alkyl)-; —S(O)₀₋₂—; C(═O); and C₁₋₃ alkylene;
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{e} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(Rr)₂; S(O)₁₋₂(C₁₋₆ alkyl); S(O)₁₋₂(C₁₋₆ haloalkyl); S(O)₁₋₂N(R^{f})₂; and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂.

In some embodiments of Formula (SI-J) and Formula (SI-K), a3 is 1. In some embodiments of Formula (SI-J) and Formula (SI-K), L^{A3} is —O—. In some embodiments of Formula (SI-J) and Formula (SI-K), a3 is 1; and L^{A3} is —O—.

In some embodiments of Formula (SI-J) and Formula (SI-K), a1a+a1b is 2 or 5. For example, a1a+a1b can be 5.

In some embodiments of Formula (SI-J) and Formula (SI-K), a1a is 0; a3b is 0 or 1; and each occurrence of L^{A1b} is —CH₂—. In some embodiments of Formula (SI-J) and Formula (SI-K), a1a is 0; a3b is 0 or 1; one occurrence of L^{A1b} is —CHR^{L}— or —C(R^{L})₂—; and each remaining occurrence of L^{A1b} is —CH₂—. In some embodiments of Formula (SI-J) and Formula (SI-K), each R^{L} is independently selected from the group consisting of: —F and —C₁₋₃ alkyl optionally substituted with 1-3 F (e.g., CH₃ or CF₃).

In some embodiments, the compounds of Formula (SI-J) are compounds of Formula (SI-J-1):

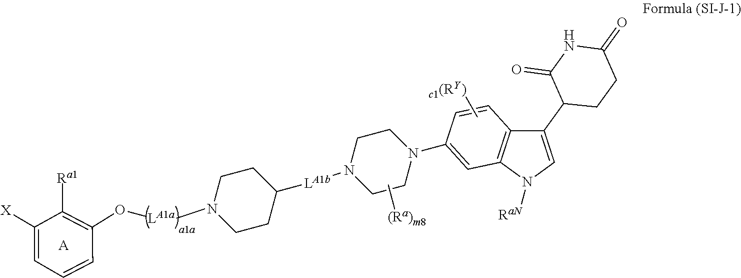

or salts thereof, wherein:
X is Br, Cl, or

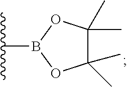 [SMILES: *B1OC(C)(C)C(C)(C)O1]

m8 is 0 or 1;
R^{a1} is C₁₋₃ alkyl optionally substituted with 1-3 F;
R^{a} is independently selected from the group consisting of: —F and —C₁₋₃ alkyl optionally substituted with 1-3 F (e.g., CH₃ or CF₃);
L^{A1a} and L^{A1b} are independently selected from the group consisting of: —CH₂—, —CHR^{L}—, and —C(R^{L})₂—;
a1a is an integer from 1 to 4;
each R^{L} is independently selected from the group consisting of: —F and —C₁₋₃ alkyl optionally substituted with 1-3 F (e.g., CH₃ or CF₃);
each R^{Y} is H or C₁₋₃ alkyl optionally substituted with 1-3 F;
c1 is 0, or 1; and
R^{aN} is C₁₋₆ alkyl optionally substituted with 1-3 F.

In some embodiments of Formula (SI-J-1), c1 is 0; m8 is 0; each occurrence of L^{A1a} is —CH₂—; and L^{A1b} is —CH₂—. For example, compounds of Formula (SI-J-1) can be:

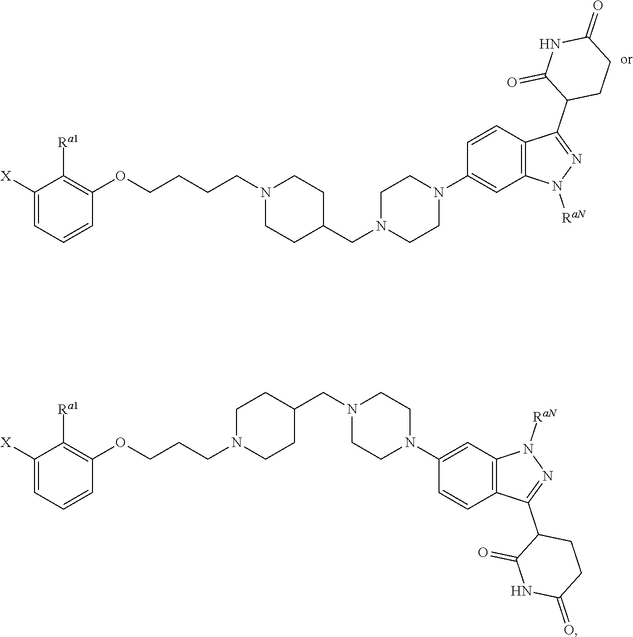

or salts thereof.

In some embodiments, the compounds of Formula (SI-J) or Formula (SI-K) are compounds of Formula (SI-J-2) or Formula (SI-K-2), respectively:

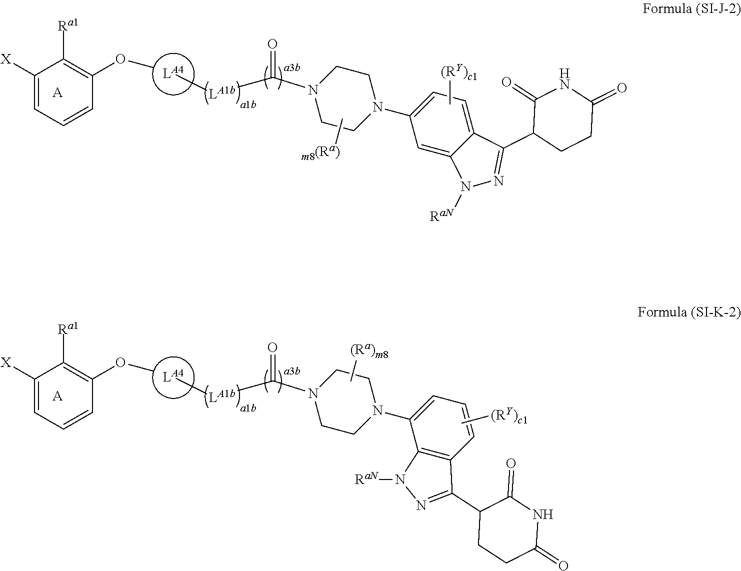

or salts thereof, wherein:
X is Br, Cl, or

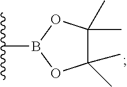 [SMILES: *B1OC(C)(C)C(C)(C)O1]

m8 is 0 or 1;
R^{a1} is C₁₋₃ alkyl optionally substituted with 1-3 F;
L^{A4} is cyclohexylene or phenylene (e.g., 1,4-cyclohexylene or 1,4-phenylene);
L^{A1b} is independently selected from the group consisting of: —CH₂—, —CHR^{L}—, and —C(R^{L})₂—;
a1b is an integer from 1 to 4;
a3b is 0 or 1;
each R^{L} is independently selected from the group consisting of: —F and —C₁₋₃ alkyl optionally substituted with 1-3 F (e.g., CH₃ or CF₃);
each R^{Y} is H or C₁₋₃ alkyl optionally substituted with 1-3 F;
c1 is 0 or 1; and
R^{aN} is C₁₋₆ alkyl optionally substituted with 1-3 F.

In some embodiments of Formula (SI-J-2) and Formula (SI-K-2), c1 is 0; m8 is 0; 0 to 1 occurrence of L^{A1b} are —CHR^{L}— or —C(R^{L})₂—; and each remaining occurrence of L^{A1b} is —CH₂—. For example, compounds of Formula (SI-J-2) and Formula (SI-K-2) can be:

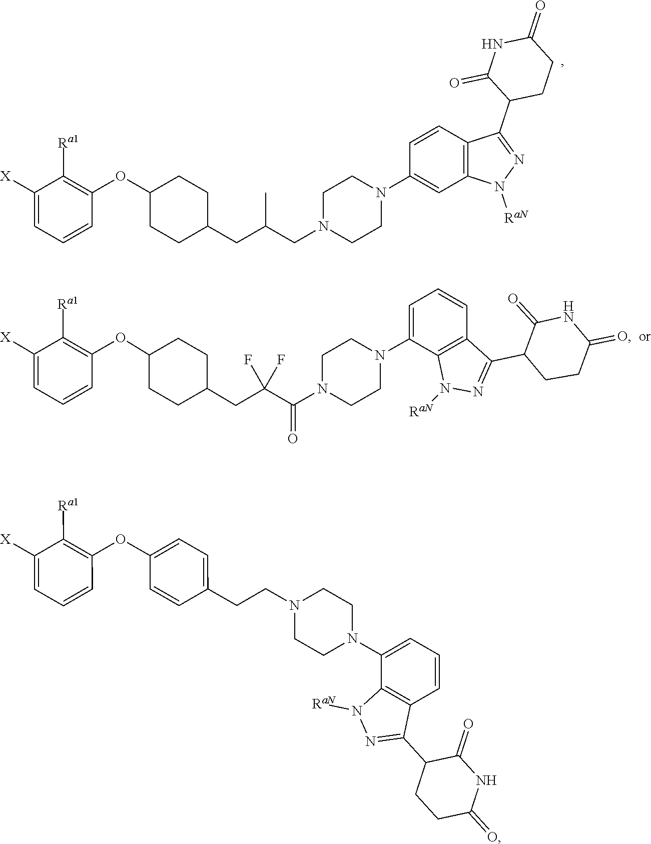

or salts thereof.

For example, compounds of Formula (SI-J-2) and Formula (SI-K-2) can be:

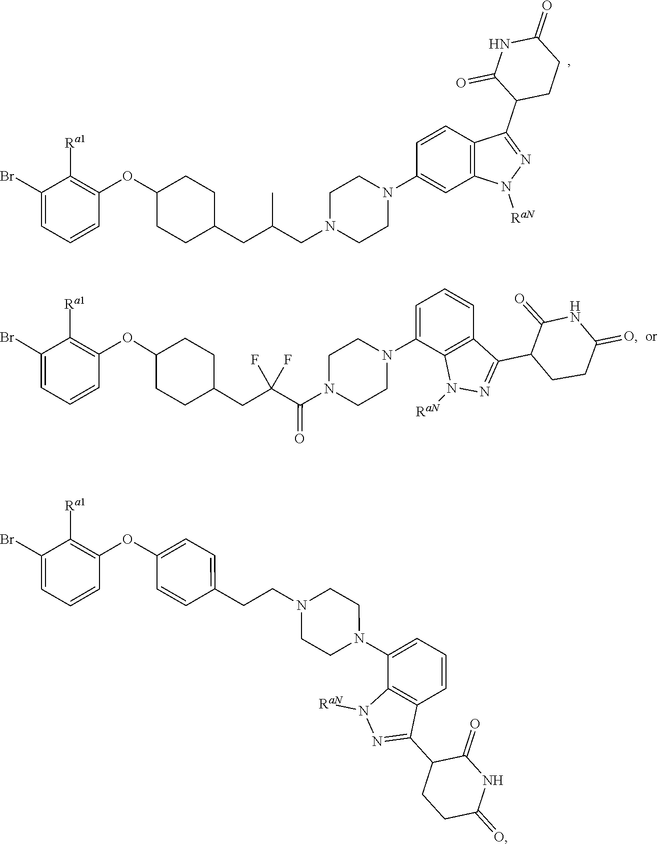

or salts thereof.

In some embodiments of Formula (SI-J-2) and Formula (SI-K-2), X is

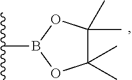 [SMILES: *B1OC(C)(C)C(C)(C)O1]

c1 is 0; m8 is 0; 0 to 1 occurrence of L^{A1b} are —CHR^{L}— or —C(R^{L})₂—; and each remaining occurrence of L^{A1b} is —CH₂—. For example, compounds of Formula (SI-J-2) and Formula (SI-K-2) can be:

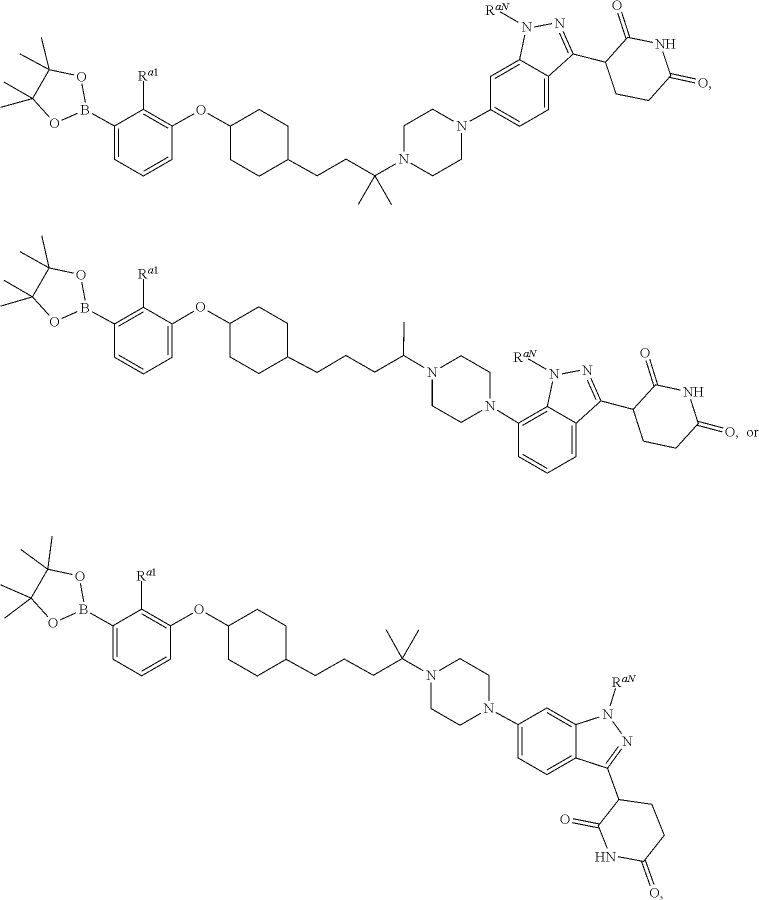

or salts thereof.

Exemplary embodiments of compounds of one or more of Formulas (SI-J), (SI-K), (SI-J-1), and (SI-J-2), and (SI-K-2) include certain compounds described in Examples 49, 58, 59, 61, 90, 101, 113, 145, 157, 172, 184, 185, 207, 208, 209, 210, and 211.

Also provided herein are methods of preparing compounds of Formula (I-A), comprising reacting compounds of Formula (SI-AA) with compounds of Formula (SII) to provide compounds of Formula (I-A), as depicted in Scheme A:

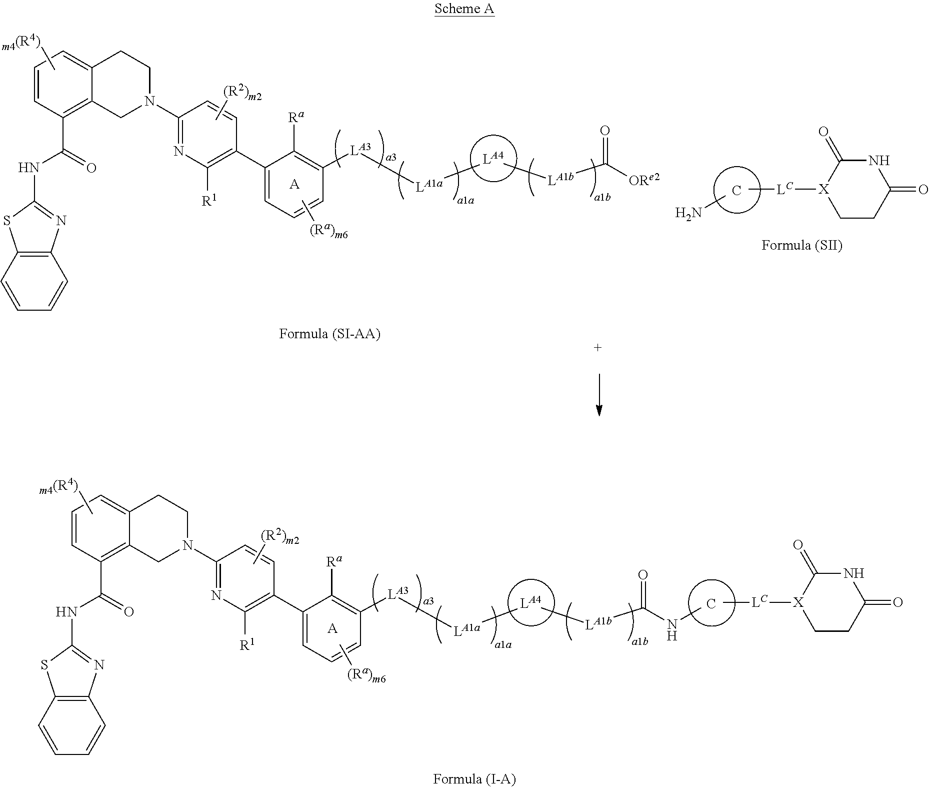

wherein:
m4 is 0, 1, 2, or 3 (e.g., 0);
m2 is 0, 1, or 2 (e.g., 0);
each R² and R⁴ is independently selected from the group consisting of: halo, CN, C₁₋₃ alkyl, C₁₋₃ haloalkyl, C₁₋₃ alkoxy, C₁₋₃ haloalkoxy, OH, and NR^{d}R^{e};
m6 is 0 or 1;
R¹ is selected from the group consisting of:
(a) C(O)OH,
(b) C(O)NR^{d}R^{e}, and
(c) C(O)OC₁₋₆ alkyl, wherein the C₁₋₆ alkyl is optionally substituted with 1-3 R^{c};
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 1 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
each R^{L} is independently selected from the group consisting of: halo; cyano; —OH; —C₁₋₆ alkoxy; —C₁₋₆ haloalkoxy; —NR^{d}R^{e}; C(═O)N(R^{f})₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{e};
R^{e2} is H or C₁₋₆ alkyl optionally substituted with 1-3-F;
Ring C is selected from the group consisting of:

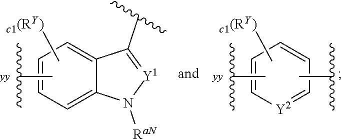

c1 is 0, 1, 2, or 3;
each R^{Y} is independently selected from the group consisting of R^{a} and R^{b};
R^{aN} is H or C₁₋₆ alkyl optionally substituted with 1-3 R^{c};
Y¹ and Y² are independently N, CH, or CR^{Y};
yy represents the point of attachment to the —C(O)NH— moiety;
X is CH or N;
L^{C} is selected from the group consisting of: a bond, —CH₂—, —CHR^{a}—, —C(R^{a})₂—, —N(R^{d})—, and O, provided that when X is N, then L^{C} is other than O;
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{C};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—; —N(H)—; —N(C₁₋₃ alkyl)-; —S(O)₀₋₂—; C(═O); and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(Rr)₂; S(O)₁₋₂(C₁₋₆ alkyl); S(O)₁₋₂(C₁₋₆ haloalkyl); S(O)₁₋₂N(R^{f})₂; and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂.

In the methods of preparing compounds of Formula (I-A) as depicted in Scheme A, the variables m4, R⁴, m2, R², R¹, R^{a}, m6, L^{A3}, a3, L^{A1a}, a1, L^{A4}, L^{A1b}, a1b, Ring C, L^{C}, and X can be as defined herein for Formula (I-A). In some embodiments of the methods, the compounds of Formula (I-A) are compounds of Formula (I-A-1). In some embodiments of the methods, the compounds of Formula (I-A) are compounds of Formula (I-A-2). In some embodiments of the methods, the compounds of Formula (I-A) are compounds of Formula (I-A-3).

In some embodiments of the methods of preparing compounds of Formula (I-A), the compounds of Formula (SI-AA) are compounds of Formula (SI-A). In some embodiments of the methods of preparing compounds of Formula (I-A), the compounds of Formula (SI-AA) are compounds of Formula (SI-A-1). In some embodiments of the methods of preparing compounds of Formula (I-A), the compounds of Formula (SI-AA) are compounds of Formula (SI-A-2). In some embodiments of the methods of preparing compounds of Formula (I-A), the compounds of Formula (SI-AA) are compounds of Formula (SI-A-3).

In some embodiments of the methods, Ring C is

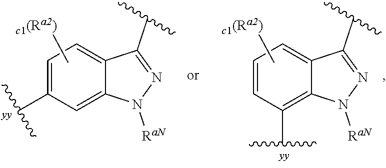

wherein: c1 is 0 or 1; R^{a2} is selected from the group consisting of: halo (e.g., —F) and C₁₋₃ alkyl optionally substituted with 1-3 F; and R^{aN} is C₁₋₃ alkyl.

In some embodiments of the methods, a3 is 1;
L^{A3} is —O—;
a1b is 1;
L^{A1b} is —CH₂—;
each L^{A1a} is independently selected from the group consisting of: —CH₂— and —CH(Me)-, provided that 0-1 L^{A1a} is —CH(Me)-; and
L^{A4} is 4-10 membered nitrogen-containing heterocyclylene optionally substituted with 1-2 substituents independently selected from the group consisting of: —F and C₁₋₃ alkyl optionally substituted with 1-3-F.

In some embodiments of the methods, the

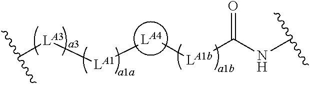

moiety is selected from the groups depicted in Table (L-I-A).

In some embodiments of the methods, R^{e2} is H; and the methods comprise reacting compounds of Formula (SI-AA) with compounds of Formula (SII) under standard conditions for amide bond formation to provide compounds of Formula (I-A). Examples of standard conditions for amide bond formation include those described in Chem. Soc. Rev., 2009, 38, 606-631, which is incorporated herein by reference in its entirety. In some embodiments, the methods comprise reacting compounds of Formula (SI-AA) with compounds of Formula (SII) in the presence of the carboxyl activating agent (e.g., HATU, HOBT, EDCI, DCC, DIC, etc.) and optionally a base (e.g., triethylamine or diisopropylethylamine) to provide compounds of Formula (I-A).

Also provided herein are methods of preparing compounds of Formula (I-B), comprising reacting compounds of Formula (SI-BB) with compounds of Formula (SII) to provide compounds of Formula (I-B), as depicted in Scheme B:

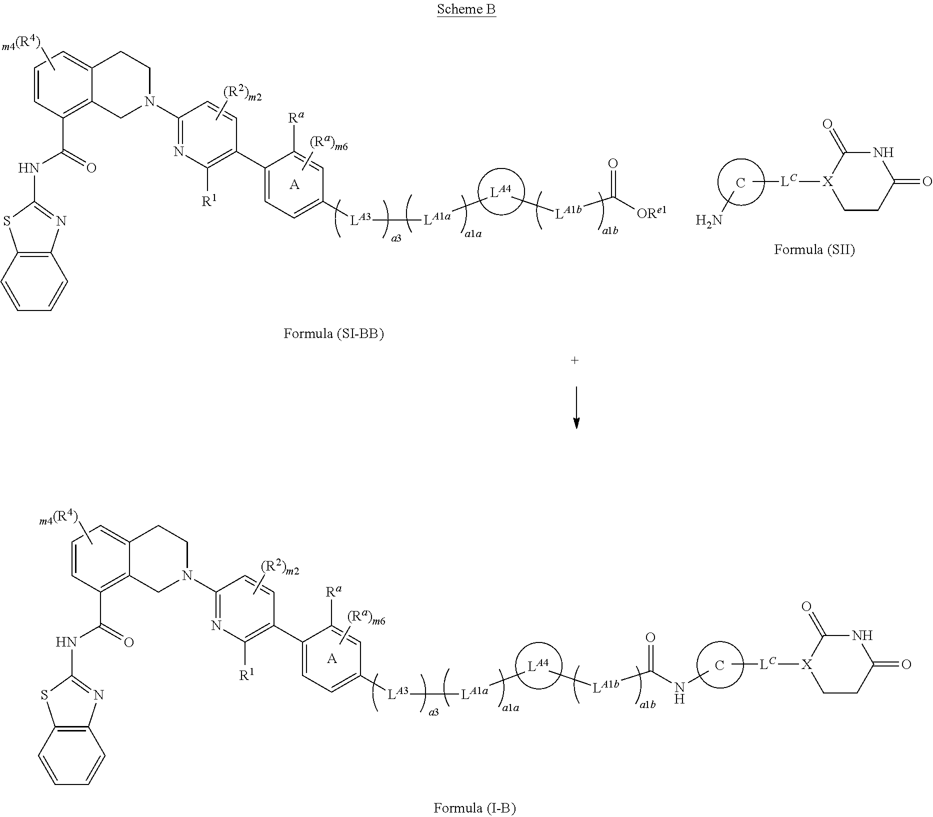

wherein:
m4 is 0, 1, 2, or 3 (e.g., 0);
m2 is 0, 1, or 2 (e.g., 0);
each R² and R⁴ is independently selected from the group consisting of: halo, CN, C₁₋₃ alkyl, C₁₋₃ haloalkyl, C₁₋₃ alkoxy, C₁₋₃ haloalkoxy, OH, and NR^{d}R^{e};
m6 is 0 or 1;
R¹ is selected from the group consisting of:
(a) C(O)OH,
(b) C(O)NR^{d}R^{e}, and
(c) C(O)OC₁₋₆ alkyl, wherein the C₁₋₆ alkyl is optionally substituted with 1-3 R^{c};
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 1 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
each R^{L} is independently selected from the group consisting of: halo; cyano; —OH; —C₁₋₆ alkoxy; —C₁₋₆ haloalkoxy; —NR^{d}R^{e}; C(═O)N(R^{f})₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{c};
R^{e2} is H or C₁₋₆ alkyl optionally substituted with 1-3-F;
Ring C is selected from the group consisting of:

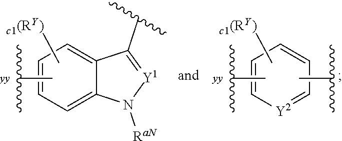

c1 is 0, 1, 2, or 3;
each R^{Y} is independently selected from the group consisting of R^{a} and R^{b};
R^{aN} is H or C₁₋₆ alkyl optionally substituted with 1-3 R^{c};
Y¹ and Y² are independently N, CH, or CR^{Y};
yy represents the point of attachment to the —C(O)NH— moiety;
X is CH or N;
L^{C} is selected from the group consisting of: a bond, —CH₂—, —CHR^{a}—, —C(R^{a})₂—, —N(R^{d})—, and O, provided that when X is N, then L^{C} is other than O;
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—; —N(H)—; —N(C₁₋₃ alkyl)-; —S(O)₀₋₂—; C(═O); and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{C} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(R^{f})₂; S(O)₁₋₂(C₁₋₆ alkyl); S(O)₁₋₂(C₁₋₆ haloalkyl); S(O)₁₋₂N(R^{f})₂; and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂.

In the methods of preparing compounds of Formula (I-B) as depicted in Scheme B, the variables m4, R⁴, m2, R², R¹, R^{a}, m6, L^{A3}, a3, L^{A1a}, a1a, L^{A4}, L^{A1b}, a1b, Ring C, L^{C}, and X can be as defined herein for Formula (I-B). In some embodiments of the methods, the compounds of Formula (I-B) are compounds of Formula (I-B-1). In some embodiments of the methods, the compounds of Formula (I-B) are compounds of Formula (I-B-2). In some embodiments of the methods, the compounds of Formula (I-B) are compounds of Formula (I-B-3).

In some embodiments of the methods of preparing compounds of Formula (I-B), the compounds of Formula (SI-BB) are compounds of Formula (SI-B). In some embodiments of the methods of preparing compounds of Formula (I-B), the compounds of Formula (SI-BB) are compounds of Formula (SI-B-1). In some embodiments of the methods of preparing compounds of Formula (I-B), the compounds of Formula (SI-BB) are compounds of Formula (SI-B-2). In some embodiments of the methods of preparing compounds of Formula (I-B), the compounds of Formula (SI-BB) are compounds of Formula (SI-B-3).

In some embodiments of the methods, Ring C is

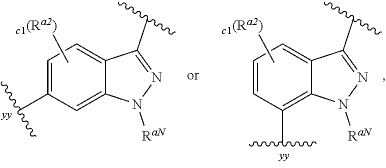

wherein: c1 is 0 or 1; R^{a2} is selected from the group consisting of: halo (e.g., —F) and C₁₋₃ alkyl optionally substituted with 1-3 F; and R^{aN} is C₁₋₃ alkyl.

In some embodiments of the methods, a3 is 1;
L^{A3} is —O—;
a1b is 1;
L^{A1b} is —CH₂—;
each L^{A1a} is independently selected from the group consisting of: —CH₂— and —CH(Me)-, provided that 0-1 L^{A1a} is —CH(Me)-; and
L^{A4} is 4-10 membered nitrogen-containing heterocyclylene optionally substituted with 1-2 substituents independently selected from the group consisting of: —F and C₁₋₃ alkyl optionally substituted with 1-3-F.

In some embodiments of the methods, the

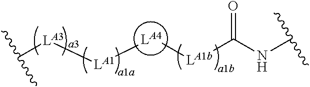

moiety is selected from the groups depicted in Table (L-I-B).

In some embodiments of the methods, R^{e2} is H; and the methods comprise reacting compounds of Formula (SI-BB) with compounds of Formula (SII) under standard conditions for amide bond formation to provide compounds of Formula (I-A). Examples of standard conditions for amide bond formation include those described in Chem. Soc. Rev., 2009, 38, 606-631, which is incorporated herein by reference in its entirety. In some embodiments, the methods comprise reacting compounds of Formula (SI-BB) with compounds of Formula (SII) in the presence of the carboxyl activating agent (e.g., HATU, HOBT, EDCI, DCC, DIC, etc.) and optionally a base (e.g., triethylamine or diisopropylethylamine) to provide compounds of Formula (I-A).

Also provided herein are methods of preparing compounds of Formula (I-E), comprising reacting compounds of Formula (SI-EE) with compounds of Formula (SIII) to provide compounds of Formula (I-E), as depicted in Scheme E:

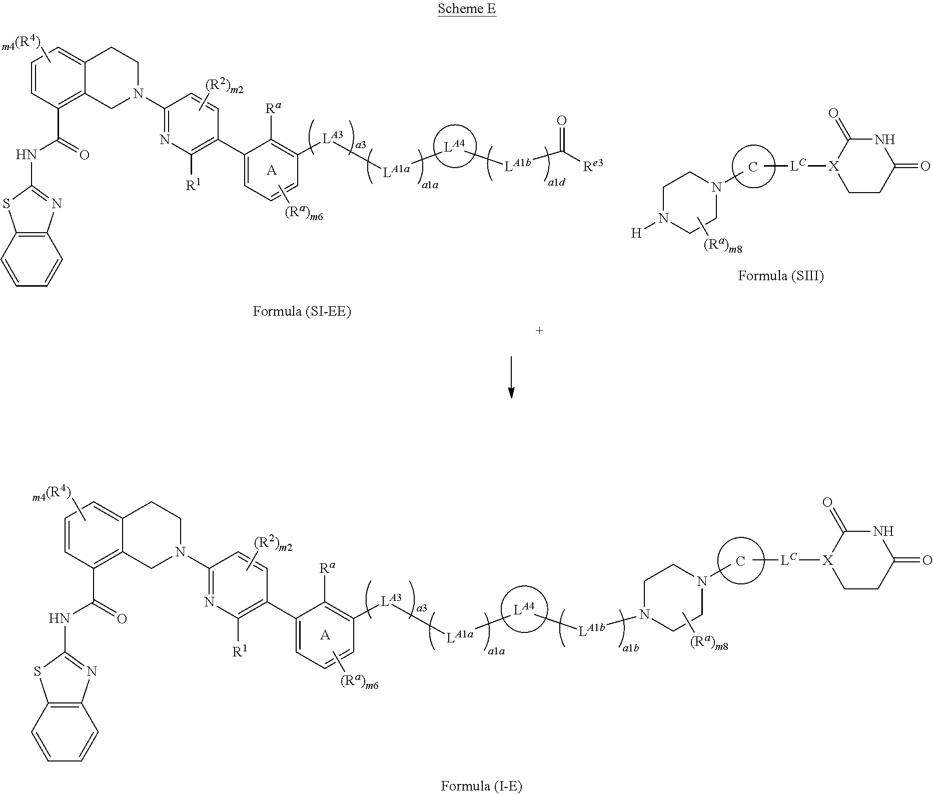

wherein:
m4 is 0, 1, 2, or 3 (e.g., 0);
m2 is 0, 1, or 2 (e.g., 0);
each R² and R⁴ is independently selected from the group consisting of: halo, CN, C₁₋₃ alkyl, C₁₋₃ haloalkyl, C₁₋₃ alkoxy, C₁₋₃ haloalkoxy, OH, and NR^{d}R^{e};
m6 is 0 or 1;
R¹ is selected from the group consisting of:
(a) C(O)OH,
(b) C(O)NR^{d}R^{e}, and
(c) C(O)OC₁₋₆ alkyl, wherein the C₁₋₆ alkyl is optionally substituted with 1-3 R^{e};
a3 is 0 or 1;
L^{A3} is selected from the group consisting of: —O—, —N(H)—, and —N(C₁₋₃ alkyl)-;
L^{A1a} and L^{A1b} are independently selected from the group consisting of: —CH₂—, —CHR^{L}—, and —C(R^{L})₂—;
a1a and a1b are independently integers from 0 to 5, provided that a1a+a1b is from 2 to 5;
a1d is an integer from 0 to 4, provided that a1a+a1d is from 1 to 4;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
each R^{L} is independently selected from the group consisting of: halo; cyano; —OH; —C₁₋₆ alkoxy; —C₁₋₆ haloalkoxy; —NR^{d}R^{e}; C(═O)N(R^{f})₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{c};
R^{e3} is H or R^{L};
m8 is 0, 1, or 2;
Ring C is selected from the group consisting of:

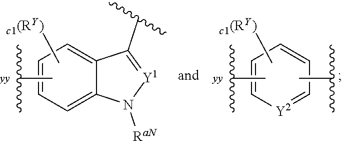

c1 is 0, 1, 2, or 3;
each R^{Y} is independently selected from the group consisting of R^{a} and R^{b};
R^{aN} is H or C₁₋₆ alkyl optionally substituted with 1-3 R^{e};
Y¹ and Y² are independently N, CH, or CR^{Y};
yy represents the point of attachment to the —C(O)NH— moiety;
X is CH or N;
L^{C} is selected from the group consisting of: a bond, —CH₂—, —CHR^{a}—, —C(R^{a})₂—, —N(R^{d})—, and O, provided that when X is N, then L^{C} is other than O;
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{C};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—; —N(H)—; —N(C₁₋₃ alkyl)-; —S(O)₀₋₂—; C(═O); and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(R^{f})₂; S(O)₁₋₂(C₁₋₆ alkyl); S(O)₁₋₂(C₁₋₆ haloalkyl); S(O)₁₋₂N(R^{f})₂; and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂.

In the methods of preparing compounds of Formula (I-E) as depicted in Scheme E, the variables m4, R⁴, m2, R², R¹, R^{a}, m6, L^{A3}, a3, L^{A1a}, a1a, L^{A4}, L^{A1b}, a1b, m8, Ring C, L^{C}, and X can be as defined herein for Formula (I-E). In some embodiments of the methods, the compounds of Formula (I-E) are compounds of Formula (I-E-1). In some embodiments of the methods, the compounds of Formula (I-E) are compounds of Formula (I-E-2). In some embodiments of the methods, the compounds of Formula (I-E) are compounds of Formula (I-E-3).

In some embodiments of the methods of preparing compounds of Formula (I-E), the compounds of Formula (SI-EE) are compounds of Formula (SI-E). In some embodiments, the compounds of Formula (SI-EE) are compounds of Formula (SI-E-1). In some embodiments, the compounds of Formula (SI-EE) are compounds of Formula (SI-E-2).

In some embodiments of the methods of preparing compounds of Formula (I-E), the compounds of Formula (SIII) are compounds of Formula (I-Ind-2) or Formula (I-Ind-3).

In some embodiments of the methods, Ring C is

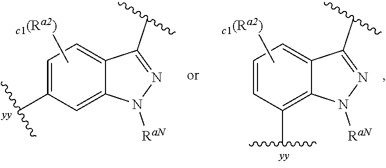

wherein: c1 is 0 or 1; R^{a2} is selected from the group consisting of: halo (e.g., —F) and C₁₋₃ alkyl optionally substituted with 1-3 F; and R^{aN} is C₁₋₃ alkyl.

In some embodiments of the methods, a3 is 1; L^{A3} is —O—; a1a is 0; and L^{A4} is selected from the group consisting of:
C₃₋₁₀ cycloalkylene optionally substituted with 1-3 R^{a}; and
phenylene optionally substituted with 1-3 R^{a}.

In some embodiments of the methods, a3 is 1; L^{A3} is —O—; a1a is 0; a1b is 3 or 4;
each L^{A1b} is independently selected from the group consisting of: —CH₂— and —CH(Me)-, provided that 0-1 of L^{A1b} is —CH(Me)-; and
L^{A4} is selected from the group consisting of:

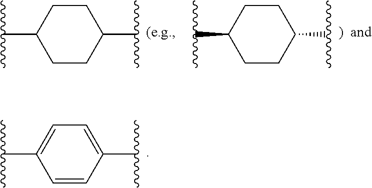 [SMILES: *C1CCC(*)CC1 ; *[C@H]1CC[C@H](*)CC1 ; *c1ccc(*)cc1]

In some embodiments of the methods, a3 is 1; L^{A3} is —O—; a1a is 2, 3, or 4; and L^{A4} is 4-10 membered heterocyclylene optionally substituted with 1-3 R^{a}.

In some embodiments of the methods, R^{e3} is H; and the methods comprising reacting compounds of Formula (SII-EE) with compounds of Formula (SIII) under standard conditions for reductive amination. In some embodiments, the methods comprise reacting compounds of Formula (SII-EE) with compounds of Formula (SIII) in the presence of a reducing agent (e.g., sodium triacetoxyborohydride, sodium cyanoborohydride, or sodium borohydride) optionally in the presence of an acid (e.g., acetic acid).

Also provided herein are methods of preparing compounds of Formula (I-E), comprising reacting compounds of Formula (SV) with compounds of Formula (SI-EE2) to provide compounds of Formula (I-E), as depicted in Scheme E2:

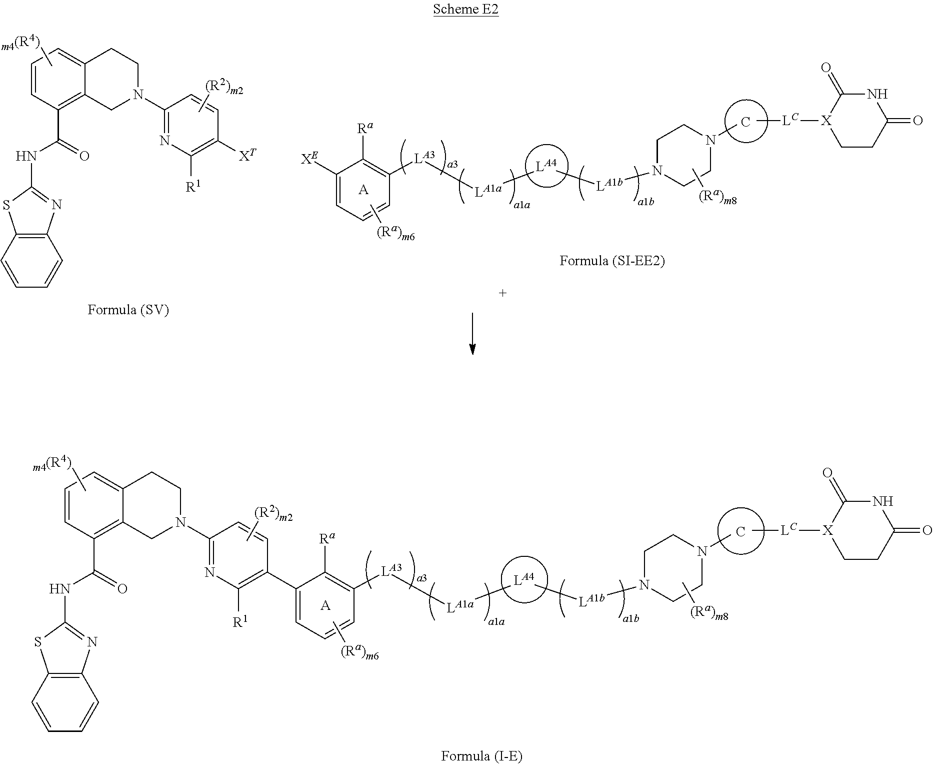

wherein:
m4 is 0, 1, 2, or 3 (e.g., 0);
m2 is 0, 1, or 2 (e.g., 0);
each R² and R⁴ is independently selected from the group consisting of: halo, CN, C₁₋₃ alkyl, C₁₋₃ haloalkyl, C₁₋₃ alkoxy, C₁₋₃ haloalkoxy, OH, and NR^{d}R^{e};
R¹ is selected from the group consisting of:
(a) C(O)OH,
(b) C(O)NR^{d}R^{e}, and
(c) C(O)OC₁₋₆ alkyl, wherein the C₁₋₆ alkyl is optionally substituted with 1-3 R^{c};
one of X^{T} and X^{E} is halo, and
the other of X^{T} and X^{E} is selected from the group consisting of:
—B(OH)₂;
—BF₃⁻;
—B(OR^{T})₂, wherein each R^{T} is independently C₁₋₃ alkyl optionally substituted with 1-3 R^{c}; and

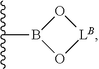

wherein L^{B} is C₂₋₆ alkylene optionally substituted with 1-4 R^{a};
m6 is 0 or 1;
a3 is 0 or 1;
L^{A3} is selected from the group consisting of: —O—, —N(H)—, and —N(C₁₋₃ alkyl)-;
L^{A1a} and L^{A1b} are independently selected from the group consisting of: —CH₂—, —CHR^{L}—, and —C(R^{L})₂—;
a1a and a1b are independently integers from 0 to 5, provided that a1a+a1b is from 2 to 5;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
each R^{L} is independently selected from the group consisting of: halo; cyano; —OH; —C₁₋₆ alkoxy; —C₁₋₆ haloalkoxy; —NR^{d}R^{e}; C(═O)N(R^{f})₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{c};
m8 is 0, 1, or 2;
Ring C is selected from the group consisting of:

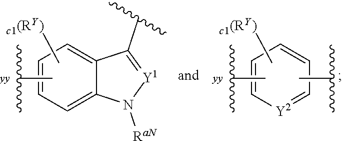

c1 is 0, 1, 2, or 3;
each R^{Y} is independently selected from the group consisting of: R^{a} and R^{b};
R^{aN} is H or C₁₋₆ alkyl optionally substituted with 1-3 R^{e};
Y¹ and Y² are independently N, CH, or CR^{Y};
yy represents the point of attachment to the —C(O)NH— moiety;
X is CH or N;
L^{C} is selected from the group consisting of: a bond, —CH₂—, —CHR^{a}—, —C(R^{a})₂—, —N(R^{d})—, and O, provided that when X is N, then L^{C} is other than O;
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{e};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—; —N(H)—; —N(C₁₋₃ alkyl)-; —S(O)₀₋₂—; C(═O); and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(Rr)₂; S(O)₁₋₂(C₁₋₆ alkyl); S(O)₁₋₂(C₁₋₆ haloalkyl); S(O)₁₋₂N(R^{f})₂; and C₁₋₆ alkyl optionally substituted with 1-3 R^{h}.
each R^{f} is independently selected from the group consisting of: H and C₁₋₆ alkyl optionally substituted with 1-3 R^{h};
each R^{g} is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂.

In the methods of preparing compounds of Formula (I-E) as depicted in Scheme E2, the variables m4, R⁴, m2, R², R¹, R^{a}, m6, L^{A3}, a3, L^{A1a}, a1a, L^{A4}, L^{A1b}, a1b, m8, Ring C, L^{C}, and X can be as defined herein for Formula (I-E). In some embodiments of the methods, the compounds of Formula (I-E) are compounds of Formula (I-E-1). In some embodiments of the methods, the compounds of Formula (I-E) are compounds of Formula (I-E-2). In some embodiments of the methods, the compounds of Formula (I-E) are compounds of Formula (I-E-3).

In some embodiments of the methods, Ring C is

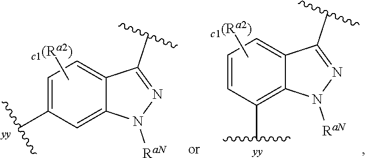

wherein: c1 is 0 or 1; R^{a2} is selected from the group consisting of: halo (e.g., —F) and C₁₋₃ alkyl optionally substituted with 1-3 F; and R^{aN} is C₁₋₃ alkyl.

In some embodiments of the methods, a3 is 1; L^{A3} is —O—; a1a is 0; and L^{A4} is selected from the group consisting of:
C₃₋₁₀ cycloalkylene optionally substituted with 1-3 R^{a}; and
phenylene optionally substituted with 1-3 R^{a}.

In some embodiments of the methods, a3 is 1; L^{A3} is —O—; a1a is 0; a1b is 3 or 4;
each L^{A1b} is independently selected from the group consisting of: —CH₂— and —CH(Me)-, provided that 0-1 of L^{A1b} is —CH(Me)-; and
L^{A4} is selected from the group consisting of:

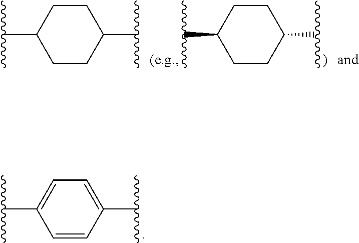 [SMILES: *C1CCC(*)CC1 ; *[C@H]1CC[C@H](*)CC1 ; *c1ccc(*)cc1]

In some embodiments of the methods, a3 is 1; L^{A3} is —O—; a1a is 2, 3, or 4; and L^{A4} is 4-10 membered heterocyclylene optionally substituted with 1-3 R^{a}.

In some embodiments of the methods, X^{T} is —B(OH)₂ or -Bpin; and X^{E} is halo.

In some embodiments of the methods, X^{T} is halo; and X^{E} is —B(OH)₂ or -Bpin.

In some embodiments of the methods, R^{e3} is H; and the methods comprising reacting compounds of Formula (SII-EE2) with compounds of Formula (SV) under standard conditions for Suzuki coupling. In some embodiments, the methods comprise reacting compounds of Formula (SII-EE2) with compounds of Formula (SV) in the presence of a palladium catalyst (e.g., CataCXium A Pd G3).

Also provided herein are methods of preparing compounds of Formula (I-Eb), comprising reacting compounds of Formula (SI-EE) with compounds of Formula (SIV) to provide compounds of Formula (I-Eb), as depicted in Scheme Eb:

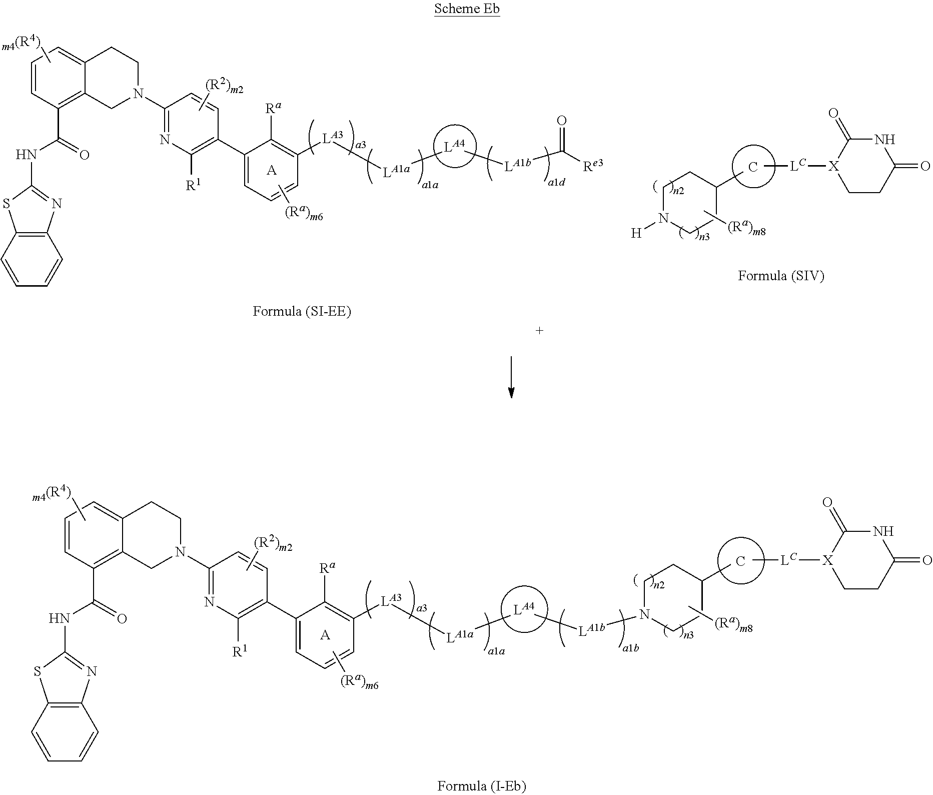

wherein:
m4 is 0, 1, 2, or 3 (e.g., 0);
m2 is 0, 1, or 2 (e.g., 0);
each R² and R⁴ is independently selected from the group consisting of: halo, CN, C₁₋₃ alkyl, C₁₋₃ haloalkyl, C₁₋₃ alkoxy, C₁₋₃ haloalkoxy, OH, and NR^{d}R^{e};
m6 is 0 or 1;
R¹ is selected from the group consisting of:
(a) C(O)OH,
(b) C(O)NR^{d}R^{e}, and
(c) C(O)OC₁₋₆ alkyl, wherein the C₁₋₆ alkyl is optionally substituted with 1-3 R^{e};
a3 is 0 or 1;
L^{A3} is selected from the group consisting of: —O—, —N(H)—, and —N(C₁₋₃ alkyl)-;
L^{A1a} and L^{A1b} are independently selected from the group consisting of: —CH₂—, —CHR^{L}—, and —C(R^{L})₂—;
a1a and a1b are independently integers from 0 to 5, provided that a1a+a1b is from 2 to 5;
a1d is an integer from 0 to 4, provided that a1a+a1d is from 1 to 4;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
each R^{L} is independently selected from the group consisting of: halo; cyano; —OH; —C₁₋₆ alkoxy; —C₁₋₆ haloalkoxy; —NR^{d}R^{e}; C(═O)N(R^{f})₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{c};
R^{c3} is H or R^{L};
n2 and n3 are independently 0, 1, or 2;
m8 is 0, 1, or 2;
Ring C is selected from the group consisting of:

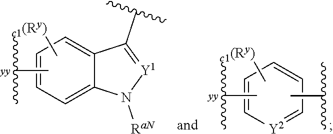

c1 is 0, 1, 2, or 3;
each R^{Y} is independently selected from the group consisting of R^{a} and R^{b};
R^{aN} is H or C₁₋₆ alkyl optionally substituted with 1-3 R^{c};
Y¹ and Y² are independently N, CH, or CR^{Y};
yy represents the point of attachment to the —C(O)NH— moiety;
X is CH or N;
L^{C} is selected from the group consisting of: a bond, —CH₂—, —CHR^{a}—, —C(R^{a})₂—, —N(R^{d})—, and O, provided that when X is N, then L^{C} is other than O;
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{c},
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—; —N(H)—; —N(C₁₋₃ alkyl)-; —S(O)₀₋₂—; C(═O); and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(R^{f})₂; S(O)₁₋₂(C₁₋₆ alkyl); S(O)₁₋₂(C₁₋₆ haloalkyl); S(O)₁₋₂N(R^{f})₂; and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂.

In the methods of preparing compounds of Formula (I-Eb) as depicted in Scheme Eb, the variables m4, R⁴, m2, R², R¹, R^{a}, m6, L^{A3}, a3, L^{A1a}, a1a, L^{A4}, L^{A1b}, a1b, n2, n3, m8, Ring C, L^{C}, and X can be as defined herein for Formula (I-Eb). In some embodiments of the methods, the compounds of Formula (I-Eb) are compounds of Formula (I-Eb-1). In some embodiments of the methods, the compounds of Formula (I-Eb) are compounds of Formula (I-Eb-2). In some embodiments of the methods, the compounds of Formula (I-Eb) are compounds of Formula (I-Eb-3).

In some embodiments of the methods of preparing compounds of Formula (I-Eb), the compounds of Formula (SI-EE) are compounds of Formula (SI-E). In some embodiments, the compounds of Formula (SI-EE) are compounds of Formula (SI-E-1). In some embodiments, the compounds of Formula (SI-EE) are compounds of Formula (SI-E-2).

In some embodiments of the methods of preparing compounds of Formula (I-Eb), the compounds of Formula (SIV) are compounds of Formula (I-Ind-1).

In some embodiments of the methods, Ring C is

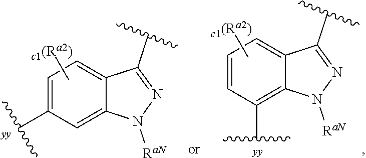

wherein: c1 is 0 or 1; R^{a2} is selected from the group consisting of: halo (e.g., —F) and C₁₋₃ alkyl optionally substituted with 1-3 F; and R^{aN} is C₁₋₃ alkyl.

In some embodiments of the methods, a3 is 1; L^{A3} is —O—; a1a is 0; and L^{A4} is selected from the group consisting of:
C₃₋₁₀ cycloalkylene optionally substituted with 1-3 R^{a}; and
phenylene optionally substituted with 1-3 R^{a}.

In some embodiments of the methods, a3 is 1; L^{A3} is —O—; a1a is 0; a1b is 3 or 4;
each L^{A1b} is independently selected from the group consisting of: —CH₂— and —CH(Me)-, provided that 0-1 of L^{A1b} is —CH(Me)-; and
L^{A4} is selected from the group consisting of:

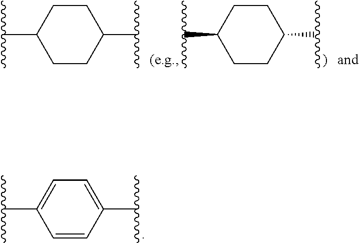 [SMILES: *C1CCC(*)CC1 ; *[C@H]1CC[C@H](*)CC1 ; *c1ccc(*)cc1]

In some embodiments of the methods, a3 is 1; L^{A3} is —O—; a1a is 2, 3, or 4; and L^{A4} is 4-10 membered heterocyclylene optionally substituted with 1-3 R^{a}.

In some embodiments of the methods, R^{e3} is H; and the methods comprising reacting compounds of Formula (SII-EE) with compounds of Formula (SIV) under standard conditions for reductive amination. In some embodiments, the methods comprise reacting compounds of Formula (SII-EE) with compounds of Formula (SIV) in the presence of a reducing agent (e.g., sodium triacetoxyborohydride, sodium cyanoborohydride, or sodium borohydride) optionally in the presence of an acid (e.g., acetic acid).

Also provided herein are methods of preparing compounds of Formula (I-Eb), comprising reacting compounds of Formula (SV) with compounds of Formula (SI-Eb2) to provide compounds of Formula (I-Eb), as depicted in Scheme Eb2:

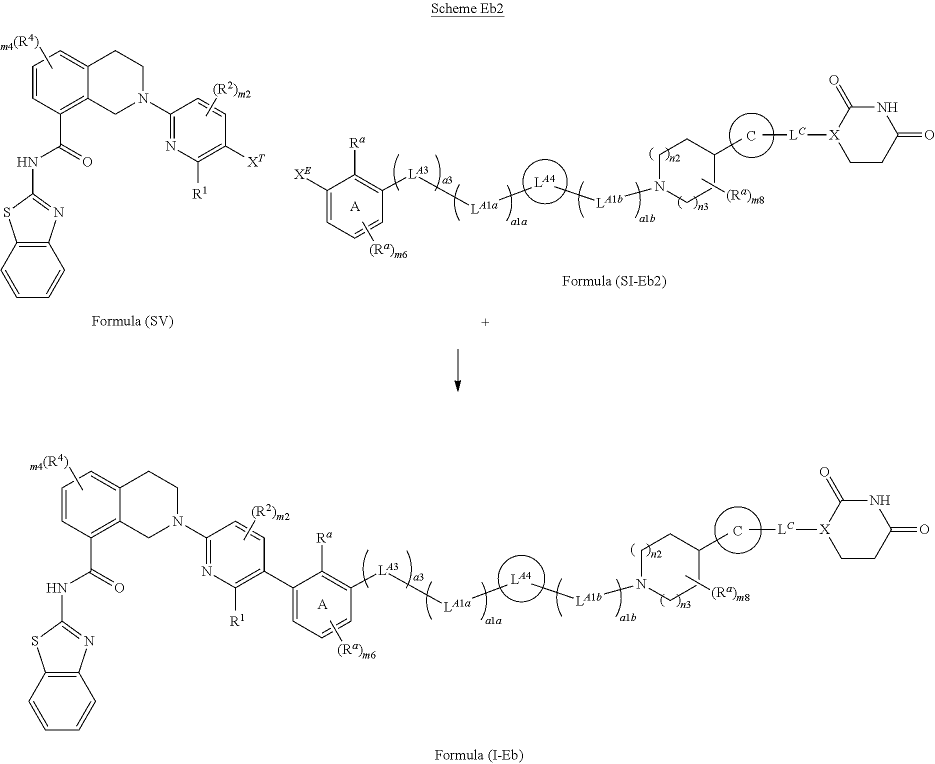

wherein:
m4 is 0, 1, 2, or 3 (e.g., 0);
m2 is 0, 1, or 2 (e.g., 0);
each R² and R⁴ is independently selected from the group consisting of: halo, CN, C₁₋₃ alkyl, C₁₋₃ haloalkyl, C₁₋₃ alkoxy, C₁₋₃ haloalkoxy, OH, and NR^{d}R^{e};
R¹ is selected from the group consisting of:
(a) C(O)OH,
(b) C(O)NR^{d}R^{e}, and
(c) C(O)OC₁₋₆ alkyl, wherein the C₁₋₆ alkyl is optionally substituted with 1-3 R^{c};
one of X^{T} and X^{E} is halo, and
the other of X^{T} and X^{E} is selected from the group consisting of:
—B(OH)₂;
—BF₃⁻;
—B(OR^{T})₂, wherein each R^{T} is independently C₁₋₃ alkyl optionally substituted with 1-3 R^{c}; and

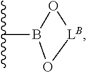

wherein L^{B} is C₂₋₆ alkylene optionally substituted with 1-4 R^{a};
m6 is 0 or 1;
a3 is 0 or 1;
L^{A3} is selected from the group consisting of: —O—, —N(H)—, and —N(C₁₋₃ alkyl)-;
L^{A1a} and L^{A1b} are independently selected from the group consisting of: —CH₂—, —CHR^{L}—, and —C(R^{L})₂—;
a1a and a1b are independently integers from 0 to 5, provided that a1a+a1b is from 2 to 5;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
each R^{L} is independently selected from the group consisting of: halo; cyano; —OH; —C₁₋₆ alkoxy; —C₁₋₆ haloalkoxy; —NR^{d}R^{e}; C(═O)N(R^{f})₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{c};
m8 is 0, 1, or 2;
Ring C is selected from the group consisting of:

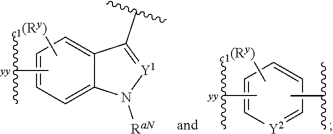

c1 is 0, 1, 2, or 3;
each R^{Y} is independently selected from the group consisting of R^{a} and R^{b};
R^{aN} is H or C₁₋₆ alkyl optionally substituted with 1-3 R^{c};
Y¹ and Y² are independently N, CH, or CR^{Y};
yy represents the point of attachment to the —C(O)NH— moiety;
X is CH or N;
L^{C} is selected from the group consisting of: a bond, —CH₂—, —CHR^{a}—, —C(R^{a})₂—, —N(R^{d})—, and O, provided that when X is N, then L^{C} is other than O;
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{e},
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—; —N(H)—; —N(C₁₋₃ alkyl)-; —S(O)₀₋₂—; C(═O); and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{C} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(R^{f})₂; S(O)₁₋₂(C₁₋₆ alkyl); S(O)₁₋₂(C₁₋₆ haloalkyl); S(O)₁₋₂N(R^{f})₂; and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂.

In the methods of preparing compounds of Formula (I-Eb) as depicted in Scheme Eb2, the variables m4, R⁴, m2, R², R¹, R^{a}, m6, L^{A3}, a3, L^{A1a}, a1a, L^{A4}, L^{A1b}, a1b, n2, n3, m8, Ring C, L^{C}, and X can be as defined herein for Formula (I-Eb). In some embodiments of the methods, the compounds of Formula (I-Eb) are compounds of Formula (I-Eb-1). In some embodiments of the methods, the compounds of Formula (I-Eb) are compounds of Formula (I-Eb-2). In some embodiments of the methods, the compounds of Formula (I-Eb) are compounds of Formula (I-Eb-3).

In some embodiments of the methods, Ring C is

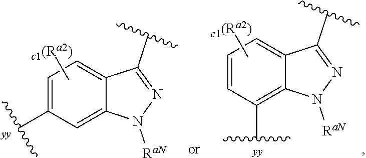

wherein: c1 is 0 or 1; R^{a2} is selected from the group consisting of: halo (e.g., —F) and C₁₋₃ alkyl optionally substituted with 1-3 F; and R^{aN} is C₁₋₃ alkyl.

In some embodiments of the methods, a3 is 1; L^{A3} is —O—; a1a is 0; and L^{A4} is selected from the group consisting of:
C₃₋₁₀ cycloalkylene optionally substituted with 1-3 R^{a}; and
phenylene optionally substituted with 1-3 R^{a}.

In some embodiments of the methods, a3 is 1; L^{A3} is —O—; a1a is 0; a1b is 3 or 4;
each L^{A1b} is independently selected from the group consisting of: —CH₂— and —CH(Me)-, provided that 0-1 of L^{A1b} is —CH(Me)-; and
L^{A4} is selected from the group consisting of:

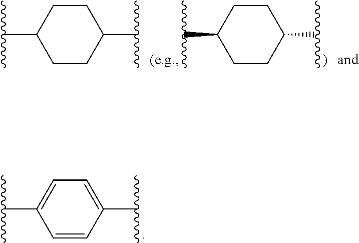 [SMILES: *C1CCC(*)CC1 ; *[C@H]1CC[C@H](*)CC1 ; *c1ccc(*)cc1]

In some embodiments of the methods, a3 is 1; L^{A3} is —O—; a1a is 2, 3, or 4; and L^{A4} is 4-10 membered heterocyclylene optionally substituted with 1-3 R^{a}.

In some embodiments of the methods, X^{T} is —B(OH)₂ or -Bpin; and X^{E} is halo.

In some embodiments of the methods, X^{T} is halo; and X^{E} is —B(OH)₂ or -Bpin.

In some embodiments of the methods, R^{e3} is H; and the methods comprising reacting compounds of Formula (SII-Eb2) with compounds of Formula (SV) under standard conditions for Suzuki coupling. In some embodiments, the methods comprise reacting compounds of Formula (SII-Eb2) with compounds of Formula (SV) in the presence of a palladium catalyst (e.g., CataCXium A Pd G3).

Also provided herein are methods of preparing compounds of Formula (I) wherein R¹ is C(O)OH, as depicted in Scheme I,
the methods comprising:
reacting compounds of Formula (I) wherein R¹ is C(O)OC₁₋₆ alkyl with an acid or a base to provide compounds of Formula (I) wherein R¹ is C(O)OH:

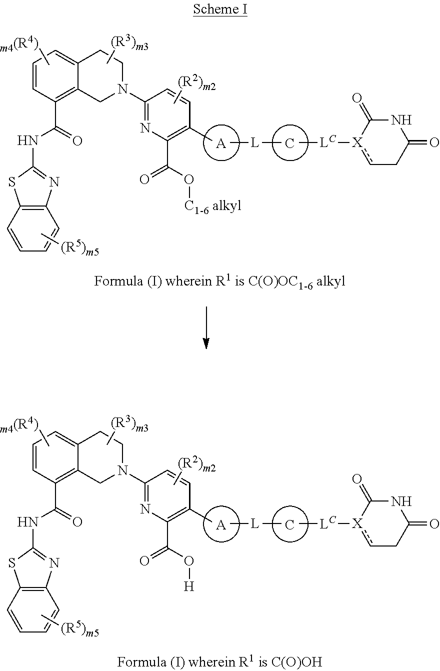

wherein:
Ring A is selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) phenylene or 5-6 membered heteroarylene, each of which is optionally substituted with 1-3 substituents independently selected from the group consisting of: R^{a} and R^{b},
each R², R³, R⁴, and R⁵ is independently selected from the group consisting of: halo, CN, C₁₋₃ alkyl, C₁₋₃ haloalkyl, C₁₋₃ alkoxy, C₁₋₃ haloalkoxy, OH, and NR^{d}R^{e};
m2 is 0, 1, or 2;
m3 and m4 are independently 0, 1, 2, or 3;
m5 is 0, 1, 2, 3, or 4;
L is -(L^{A})ₙ₁-, wherein L^{A} and n1 are defined according to (AA) or (BB):
   (AA)
n1 is an integer from 3 to 15; and
each L^{A} is independently selected from the group consisting of: L^{A1} L^{A3}, and L^{A4}, provided that 1-3 occurrences of L^{A} is L^{A4};
   (BB)
n1 is an integer from 0 to 20; and
each L^{A} is independently selected from the group consisting of: L^{A1} and L^{A3};
each L^{A1} is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b} and
(b) C₆₋₁₅ arylene or 5-15 membered heteroarylene, each of which is optionally substituted with 1-6 substituents independently selected from the group consisting of: R^{a} and R^{b};
provided that L does not contain any N—O, O—O, N—N, N—S(O)₀, or O—S(O)₀₋₂ bonds;
wherein each R^{L} is independently selected from the group consisting of: halo; cyano; —OH; —C₁₋₆ alkoxy; —C₁₋₆ haloalkoxy; —NR^{d}R^{e}; C(═O)N(R′)₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{c};
Ring C is selected from the group consisting of:

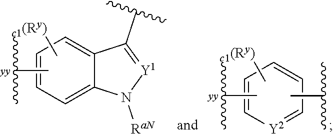

c1 is 0, 1, 2, or 3;
each R^{Y} is independently selected from the group consisting of R^{a} and R^{b};
R^{aN} is H or C₁₋₆ alkyl optionally substituted with 1-3 R^{c};
Y¹ and Y² are independently N, CH, or CR^{Y};
yy represents the point of attachment to L;
X is CH, C, or N;
the [symbol] is a single bond or a double bond;
L^{C} is selected from the group consisting of: a bond, —CH₂—, —CHR^{a}—, —C(R^{a})₂—, —N(R^{d})—, and O, provided that when X is N, then L^{C} is other than O;
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{c};
each R^{b} is independently selected from the group consisting of: -(L^{h})_{b}-R^{b1} and —R^{b1}, wherein:
each b is independently 1, 2, or 3;
each -L^{b} is independently selected from the group consisting of: —O—; —N(H)—; —N(C₁₋₃ alkyl)-; —S(O)₀₋₂—; C(═O); and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(R^{f})₂; S(O)₁₋₂(C₁₋₆ alkyl); S(O)₁₋₂(C₁₋₆ haloalkyl); S(O)₁₋₂N(R^{f})₂; and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂.

In the methods of preparing compounds of Formula (I) wherein R¹ is C(O)OH, as depicted in Scheme I, m5, R⁵, m4, R⁴, m3, R³, m2, R², Ring A, L, Ring C, L^{C}, and X can be as defined for Formula (I) herein. In some embodiments of the methods, L is (L-1). In some embodiments of the methods, L is (L-2). In some embodiments of the methods, L is (L-3). In some embodiments of the methods, L is (L-3a). In some embodiments of the methods, L is (L-3b). In some embodiments of the methods, L is (L-3c). In some embodiments of the methods, L is (L-3d). In some embodiments of the methods, L is (L-4).

In some embodiments, the methods provide compounds of Formula (I-A), wherein R¹ is C(O)OH. In some embodiments, the methods provide compounds of Formula (I-B), wherein R¹ is C(O)OH. In some embodiments, the methods provide compounds of Formula (I-C), wherein R¹ is C(O)OH. In some embodiments, the methods provide compounds of Formula (I-D), wherein R¹ is C(O)OH. In some embodiments, the methods provide compounds of Formula (I-E), wherein R¹ is C(O)OH. In some embodiments, the methods provide compounds of Formula (I-Ea), wherein R¹ is C(O)OH. In some embodiments, the methods provide compounds of Formula (I-Eb), wherein R¹ is C(O)OH. In some embodiments, the methods provide compounds of Formula (I-F), wherein R¹ is C(O)OH. In some embodiments, the methods provide compounds of Formula (I-G), wherein R¹ is C(O)OH.

In some embodiments, the method comprising reacting compounds of Formula (I) wherein R¹ is C(O)O^{t}Bu with an acid (e.g., TFA or HCl) to provide compounds of Formula (I) wherein R¹ is C(O)OH.

### EXAMPLES

In some of the examples disclosed herein, the final product of a described chemical reaction sequence is structurally depicted with an enhanced stereochemical or1 notation at one stereogenic center. In some such examples, in the chemical name of the same compound, this stereogenic center is assigned a tentative configuration (e.g., (R)- or (S)-) based on the wedge/dash representation of the structural formula. However, this stereogenic center should be understood to have a configuration consistent with the or1 notation. Specifically, this stereogenic center has been resolved, but its specific configuration has not been determined. Accordingly, unless otherwise specified, starting materials and intermediates leading to this compound incorporate the or1 notation at this stereogenic center, notwithstanding the tentative assignments provided in their chemical names.

For example, Compound 289a in Example 116 is a single stereoisomer selected from:
6-(8-(benzo[d]thiazol-2-ylcarbamoyl)-3,4-dihydroisoquinolin-2(1H)-yl)-3-(3-(2-((1S)-6-(2-((3-(2,6-dioxopiperidin-3-yl)-1-methyl-1H-indazol-6-yl)amino)-2-oxoethyl)-6-azaspiro[2.5]octan-1-yl)ethoxy)-2-methylphenyl)picolinic acid; and
6-(8-(benzo[d]thiazol-2-ylcarbamoyl)-3,4-dihydroisoquinolin-2(1H)-yl)-3-(3-(2-((1R)-6-(2-((3-(2,6-dioxopiperidin-3-yl)-1-methyl-iH-indazol-6-yl)amino)-2-oxoethyl)-6-azaspiro[2.5]octan-1-yl)ethoxy)-2-methylphenyl)picolinic acid.

The intermediate product provided in Step A of the same example incorporates the or1 notation. It is therefore a single stereoisomer selected from:
tert-butyl (S)-6-(8-(benzo[d]thiazol-2-ylcarbamoyl)-3,4-dihydroisoquinolin-2(1H)-yl)-3-(3-(2-(6-(2-ethoxy-2-oxoethyl)-6-azaspiro[2.5]octan-1-yl)ethoxy)-2-methylphenyl)picolinate; and
tert-butyl (R)-6-(8-(benzo[d]thiazol-2-ylcarbamoyl)-3,4-dihydroisoquinolin-2(1H)-yl)-3-(3-(2-(6-(2-ethoxy-2-oxoethyl)-6-azaspiro[2.5]octan-1-yl)ethoxy)-2-methylphenyl)picolinate.

For further clarification, a chemical name that takes into account the or1 notation is provided for the final product having an enhanced stereochemical or1 notation at one stereogenic center. This chemical name is enclosed in brackets (i.e., “[ ]”). In these chemical names, the prefix “rel” means that the stereochemical configuration shown in a chemical name is relative. To illustrate, when a compound contains one stereogenic center, and its chemical name starts with the prefix “rel,” then this stereogenic center is resolved, but its absolute configuration is either (R)- or (S)-. As such, it should be labelled with an or1 enhanced stereochemical notation in its corresponding structure.

For example, the chemical name that takes into account the or1 notation for Compound 289a is:
[rel-6-(8-(benzo[d]thiazol-2-ylcarbamoyl)-3,4-dihydroisoquinolin-2(1H)-yl)-3-(3-(2-((1S)-6-(2-((3-(2,6-dioxopiperidin-3-yl)-1-methyl-1H-indazol-6-yl)amino)-2-oxoethyl)-6-azaspiro[2.5]octan-1-yl)ethoxy)-2-methylphenyl)picolinic acid], as included in Example 116.

When a stereogenic center is labelled with an asterisk (“*”) in a chemical name of a compound having more than one stereogenic center, the stereogenic center labeled with the asterisk is resolved, but its absolute configuration is either (R)- or (S)—. As such, in a chemical name wherein one stereogenic center is labelled with the asterisk, that stereocenter should be labelled with an or1 enhanced stereochemical notation in its corresponding structure.

For example, the chemical name containing an asterisk for Compound 282b is:

[6-(8-(benzo[d]thiazol-2-ylcarbamoyl)-3,4-dihydroisoquinolin-2(1H)-yl)-3-(3-(((1S,4r)-4-((2S*)-3-(4-(3-(2,6-dioxopiperidin-3-yl)-1-methyl-1H-indazol-6-yl)piperazin-1-yl)-2-methylpropyl)cyclohexyl)oxy)-2-methylphenyl)picolinic acid], as included in Example 185.

### Example 1. Preparation of Compound 121

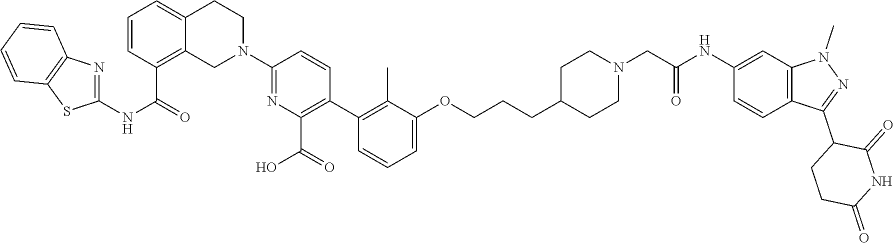 [SMILES: Cc1c(OCCCC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-(8-(Benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-6-yl)amino)-2-oxoethyl)piperidin-4-yl)propoxy)-2-methylphenyl)picolinic acid

### Method 1

### Step A. tert-Butyl 4-(3-(3-bromo-2-methylphenoxy)propyl)piperidine-1-carboxylate

Combined 3-bromo-2-methylphenol (1 equiv.), tert-butyl 4-(3-bromopropyl)piperidine-1-carboxylate (1 equiv.), and potassium carbonate (3 equiv.) in DMF and heated at 50° C. for 16 h. Diluted reaction mixture with ethyl acetate and brine. Separated layers, dried organic layer over anhydrous magnesium sulfate, filtered, and concentrated to an oil that was purified by silica gel chromatography (heptane/ethyl acetate gradient). Combined and concentrated product containing fractions to afford tert-butyl 4-(3-(3-bromo-2-methylphenoxy)propyl)piperidine-1-carboxylate as an oil that was used without further purification assuming theoretical yield. MS (ESI) m/z: 412.2 [M+H]⁺.

### Step B. 4-(3-(3-Bromo-2-methylphenoxy)propyl)piperidine, HCl

Combined tert-butyl 4-(3-(3-bromo-2-methylphenoxy)propyl)piperidine-1-carboxylate (1 equiv.) and 4 M HCl in dioxane (10 equiv.) in DCM and let mix under ambient temperature for 40 h. Concentrated mixture to dryness, triturated residue with diethyl ether and hexanes, re-evaporated, and dried the resulting solids overnight under vacuum to afford 4-(3-(3-bromo-2-methylphenoxy)propyl)piperidine, HCl. MS (ESI) m/z: 312.2 [M+H]⁺.

### Step C. Methyl 2-(4-(3-(3-bromo-2-methylphenoxy)propyl)piperidin-1-yl)acetate

Combined 4-(3-(3-bromo-2-methylphenoxy)propyl)piperidine, HCl (1 equiv.), methyl 2-bromoacetate (1 equiv.), and potassium carbonate (4 equiv.) in DMF and heated overnight at 50° C. for 16 h. Diluted reaction mixture with ethyl acetate and brine. Separated layers, dried organic over anhydrous magnesium sulfate, filtered, and concentrated to an oil that was purified by silica gel chromatography (heptane/ethyl acetate (w/ 10% of a 7N NH₃ solution in methanol)). Combined and concentrated product containing fractions to afford methyl 2-(4-(3-(3-bromo-2-methylphenoxy)propyl)piperidin-1-yl)acetate. MS (ESI) m/z: 384.2 [M+H]⁺.

### Step D. Methyl 2-(4-(3-(2-methyl-3-(4,4,5,5-tetramethyl-1,3,2-dioxaborolan-2-yl)phenoxy)propyl)piperidin-1-yl)acetate

Methyl 2-(4-(3-(3-bromo-2-methylphenoxy)propyl)piperidin-1-yl)acetate (1 equiv.), bis(pinacolato)diboron (1.7 equiv.), and PdCl₂(dppf)-CH₂Cl₂adduct (0.1 equiv.) were combined in 1,4-dioxane. Potassium acetate (3 equiv.) was then added, and the mixture was stirred at 80° C. for 16 h. Diluted reaction mixture with ethyl acetate, water, and brine. Separated layers and washed organic layer with an additional brine. Dried organic layer over anhydrous magnesium sulfate, filtered, and concentrated to dryness under reduced pressure. Purified residue on silica gel (heptane/ethyl acetate gradient). Product containing fractions were combined, concentrated, and dried under vacuum to afford methyl 2-(4-(3-(2-methyl-3-(4,4,5,5-tetramethyl-1,3,2-dioxaborolan-2-yl)phenoxy)propyl)piperidin-1-yl)acetate which was used without further purification. MS (ESI) m/z: 432.4 [M+H]⁺.

### Step E. tert-Butyl 6-(8-(benzo[d]thiazol-2-ylcarbamoyl)-3,4-dihydroisoquinolin-2(1H)-yl)-3-(3-(3-(1-(2-methoxy-2-oxoethyl)piperidin-4-yl)propoxy)-2-methylphenyl)picolinate

Combined tert-butyl 6-(8-(benzo[d]thiazol-2-ylcarbamoyl)-3,4-dihydroisoquinolin-2(1H)-yl)-3-bromopicolinate and methyl 2-(4-(3-(2-methyl-3-(4,4,5,5-tetramethyl-1,3,2-dioxaborolan-2-yl)phenoxy)propyl)piperidin-1-yl)acetate in 1,4-dioxane. Added potassium phosphate (4 equiv.) and CataCxium Pd G3 (0.05 equiv.). Let mixture stir at 100° C. for 1 hr under microwave heating. Diluted reaction mixture with ethyl acetate, water, and brine. Separated layers and washed organic layer with an additional brine. Dried over anhydrous magnesium sulfate, filtered, and concentrated to dryness under reduced pressure to afford tert-butyl 6-(8-(benzo[d]thiazol-2-ylcarbamoyl)-3,4-dihydroisoquinolin-2(1H)-yl)-3-(3-(3-(1-(2-methoxy-2-oxoethyl)piperidin-4-yl)propoxy)-2-methylphenyl)picolinate which was used without further purification assuming theoretical yield.

MS (ESI) m/z: 790.4 [M+H]⁺.

### Step F. 2-(4-(3-(3-(6-(8-(Benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)acetic acid

tert-Butyl 6-(8-(benzo[d]thiazol-2-ylcarbamoyl)-3,4-dihydroisoquinolin-2(1H)-yl)-3-(3-(3-(1-(2-methoxy-2-oxoethyl)piperidin-4-yl)propoxy)-2-methylphenyl)picolinate (1 equiv.) was dissolved in a mixture of THF, methanol, and water. Lithium hydroxide (10 equiv.) was added and the mixture was stirred under ambient temperature for 16 h. The pH was adjusted to ˜5-6 with formic acid and the volatiles were removed under reduced pressure. The residue was triturated in water to provide solids that were filtered, rinsed with water, and dried under vacuum to afford 2-(4-(3-(3-(6-(8-(Benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)acetic acid that was used without further purification. MS (ESI) m/z: 776.4 [M+H]⁺.

### Step G. tert-Butyl 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-6-yl)amino)-2-oxoethyl)piperidin-4-yl)propoxy)-2-methylphenyl)picolinate

2-(4-(3-(3-(6-(8-(Benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)acetic acid (1 equiv.) was dissolved in a mixture of DMF and DIEA (5 equiv.). HATU (1.2 equiv.) was added to the solution and let mix at ambient temperature for 5 min. 3-(6-Amino-1-methyl-1H-indazol-3-yl)piperidine-2,6-dione (1 equiv.) was added to the solution and the mixture was stirred at ambient temperature for 16 h. Additional amounts of DIEA and HATU (⅓ amount originally used) to the reaction mixture and stirred for 5 min. Added 150 mg additional of 3-(6-amino-1-methyl-1H-indazol-3-yl)piperidine-2,6-dione (1 equiv.) to the solution and let continue to mix at ambient temperature for 1 h. Diluted reaction mixture with ethyl acetate and brine. Separated layers, dried organic over anhydrous magnesium sulfate, filtered, and concentrated to an oil affording tert-butyl 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-6-yl)amino)-2-oxoethyl)piperidin-4-yl)propoxy)-2-methylphenyl)picolinate that was used without further purification assuming theoretical yield. MS (ESI) m/z: 1016.4 [M+H]⁺.

### Step H. 6-(8-(Benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-6-yl)amino)-2-oxoethyl)piperidin-4-yl)propoxy)-2-methylphenyl)picolinic acid

Dissolved tert-butyl 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-6-yl)amino)-2-oxoethyl)piperidin-4-yl)propoxy)-2-methylphenyl)picolinate (1 equiv.) in a mixture of DCM and trifluoroacetic acid (50 equiv.). Let reaction stir for 16 h at ambient temperature. Concentrated reaction mixture to dryness under reduced pressure and purified by preparative RP-HPLC (water/acetonitrile, 0.1% formic acid in each). Combined and lyophilized pure product containing fractions to afford 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-6-yl)amino)-2-oxoethyl)piperidin-4-yl)propoxy)-2-methylphenyl)picolinic acid. MS (ESI) m/z: 960.4 [M+H]⁺; H NMR (500 MHz, DMSO) δ 12.87 (s, 1H), 10.88 (s, 1H), 9.97-9.63 (m, 1H), 8.05-8.00 (m, 2H), 7.81-7.76 (m, 1H), 7.65-7.59 (m, 2H), 7.50-7.42 (m, 3H), 7.41-7.31 (m, 2H), 7.24-7.15 (m, 1H), 7.13-7.06 (m, 1H), 7.00-6.94 (m, 1H), 6.91-6.85 (m, 1H), 6.65-6.60 (m, 1H), 4.98 (s, 2H), 4.36-4.29 (m, 1H), 3.99-3.94 (m, 2H), 3.92 (s, 5H), 3.14-3.11 (m, 2H), 3.06-3.00 (m, 2H), 2.90-2.87 (m, 2H), 2.68-2.56 (m, 1H), 2.40-2.07 (m, 1H), 1.90 (s, 3H), 1.82-1.60 (m, 5H), 1.48-1.02 (m, 8H).

### Method 2

### Step A. tert-butyl 4-(3-(3-bromo-2-methylphenoxy)propyl)piperidine-1-carboxylate

A mixture of 3-bromo-2-methyl-phenol (2 g, 10.69 mmol, 1 equiv.), K₂CO₃ (4.43 g, 32.08 mmol, 3 eq.), tert-butyl 4-(3-hydroxypropyl)piperidine-1-carboxylate (3.27 g, 10.69 mmol, 1 equiv.) in MeCN (20 mL) was degassed and purged with N₂ three times. The mixture was stirred at 60° C. for 12 hours under N₂ atmosphere. The reaction mixture was concentrated under reduced pressure to give a residue. The residue was purified by flash silica gel chromatography (0-7% ethyl acetate/petroleum ether) to give tert-butyl 4-(3-(3-bromo-2-methylphenoxy)propyl)piperidine-1-carboxylate (1.8 g, 4.37 mmol, 40.82% yield) as a colorless oil.

¹H NMR (400 MHz, CDCl₃) δ=7.09-7.07 (m, 1H), 6.91 (t, J=8.0 Hz, 1H), 6.68 (s, 1H), 3.86 (t, J=6.4 Hz, 2H), 2.63 (t, J=12.0 Hz, 2H), 2.23 (s, 3H), 1.79-1.69 (m, 2H), 1.69-1.50 (m, 4H), 1.39 (s, 9H), 1.38-1.30 (m, 3H), 1.11-0.98 (m, 2H)

### Step B. 4-(3-(3-bromo-2-methylphenoxy)propyl)piperidine

A mixture of tert-butyl 4-(3-(3-bromo-2-methylphenoxy)propyl)piperidine-1-carboxylate (1.8 g, 4.37 mmol, 1 equiv.) in HCl/EtOAc (20 mL) was stirred at 20° C. for 1 hour. The reaction mixture was concentrated under reduced pressure to give 4-(3-(3-bromo-2-methylphenoxy)propyl)piperidine (1.5 g, crude) as a white solid.

MS (ESI) m/z: 313.9 [M+3]⁺.

### Step C. Ethyl 2-(4-(3-(3-bromo-2-methylphenoxy)propyl)piperidin-1-yl)acetate

A mixture of 4-(3-(3-bromo-2-methylphenoxy)propyl)piperidine (1 g, 3.20 mmol, 1 equiv.), ethyl 2-bromoacetate (534.84 mg, 3.20 mmol, 354.20 uL, 1 equiv.), and K₂CO₃ (885.24 mg, 6.41 mmol, 2 equiv.) in MeCN (15 mL) was degassed and purged with N₂ three times. The mixture was stirred at 60° C. for 2 hours under N₂ atmosphere. The reaction mixture was concentrated under reduced pressure to give a residue. The residue was purified by flash silica gel chromatography (0˜45% ethyl acetate/petroleum ether) to give ethyl 2-(4-(3-(3-bromo-2-methylphenoxy)propyl)piperidin-1-yl)acetate (0.5 g, 1.26 mmol, 39.19% yield) as a yellow oil

MS (ESI) m/z: 399.9 [M+3]⁺.

¹H NMR (400 MHz, CD₃OD) δ=7.14 (d, J=8.0 Hz, 1H), 6.98 (t, J=8.0 Hz, 1H), 6.75 (d, J=8.0 Hz, 1H), 4.19 (q, J=7.2 Hz, 2H), 3.93 (t, J=6.4 Hz, 2H), 3.23-3.18 (m, 2H), 2.95 (d, J=11.6 Hz, 2H), 2.31 (s, 3H), 2.18-2.11 (m, 2H), 1.84-1.79 (m, 2H), 1.72-1.64 (m, 4H), 1.59-1.53 (m, 2H), 1.46-1.40 (m, 4H), 1.35 (dd, J=3.2, 10.8 Hz, 2H), 1.28 (t, J=7.2 Hz, 3H)

### Step D. tert-Butyl 6-(8-(benzo[d]thiazol-2-ylcarbamoyl)-3,4-dihydroisoquinolin-2(1H)-yl)-3-(3-(3-(1-(2-ethoxy-2-oxoethyl)piperidin-4-yl)propoxy)-2-methylphenyl)picolinate

A mixture of ethyl 2-(4-(3-(3-bromo-2-methylphenoxy)propyl)piperidin-1-yl)acetate (500 mg, 1.26 mmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (768.89 mg, 1.26 mmol, 1 equiv.), [2-(2-aminophenyl)phenyl]palladium(1+); bis(1-adamantyl)-butyl-phosphane; methanesulfonate (91.41 mg, 125.52 umol, 0.1 equiv.), and K₃PO₄ (1.5 M, 2.51 mL, 3 equiv.) in 1,4-dioxane (5 mL) was degassed and purged with N₂ three times. The mixture was stirred at 80° C. for 12 hours under N₂ atmosphere. The reaction mixture was concentrated under reduced pressure to give a residue. The residue was purified by flash silica gel chromatography (0˜5% ethyl acetate/petroleum ether) to give tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)piperidin-4-yl)propoxy)-2-methylphenyl)picolinate (800 mg, 696.51 umol, 55.49% yield, 70% purity) as a yellow solid.

MS (ESI) m/z: 804.5 [M+H]⁺.

### Step E. 2-(4-(3-(3-(6-(8-(Benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)acetic acid

To a solution of tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)piperidin-4-yl)propoxy)-2-methylphenyl)picolinate (342.86 mg, 298.51 umol, 70% purity, 1 equiv.) in THE (3 mL) was added LiOH (1 M, 895.52 uL, 3 equiv.). The mixture was stirred at 20° C. for 1 hour. The reaction mixture was adjusted pH to 5, filtered, and concentrated under reduced pressure to give 2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)acetic acid (150 mg, 193.31 umol, 64.76% yield) as a yellow solid.

MS (ESI) m/z: 776.6 [M+H]⁺.

### Step F. tert-Butyl 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-6-yl)amino)-2-oxoethyl)piperidin-4-yl)propoxy)-2-methylphenyl)picolinate

A mixture of 2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)acetic acid (70 mg, 90.21 umol, 1 equiv.), 3-(6-amino-1-methyl-1H-indazol-3-yl)piperidine-2,6-dione (23.30 mg, 90.21 umol, 1 equiv.), HATU (37.73 mg, 99.23 umol, 1.1 equiv.), and TEA (27.39 mg, 270.64 umol, 37.67 uL, 3 equiv.) in DMF (1 mL) was degassed and purged with N₂ three times. The mixture was stirred at 25° C. for 12 hours under N₂ atmosphere. The solution was poured into water (2 mL), filtered, and concentrated under reduced pressure to give tert-butyl 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-6-yl)amino)-2-oxoethyl)piperidin-4-yl)propoxy)-2-methylphenyl)picolinate (60 mg, 59.04 umol, 65.45% yield) as a yellow solid.

MS (ESI) m/z: 508.9 [M12+H]⁺.

### Step G. 6-(8-(Benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-6-yl)amino)-2-oxoethyl)piperidin-4-yl)propoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-6-yl)amino)-2-oxoethyl)piperidin-4-yl) propoxy)-2-methylphenyl)picolinate (60 mg, 59.04 umol, 1 equiv.) in DCM (0.5 mL) was added TFA (0.5 mL). The mixture was stirred at 20° C. for 0.5 hours. The reaction mixture was concentrated under reduced pressure to give a residue. The residue was purified by prep-HPLC (column: Phenomenex luna C18 150×25 mm×10 um; mobile phase: [water (formic acid)-MeCN]; B %: 27%-57%, 15 min) to give 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-iH-indazol-6-yl)amino)-2-oxoethyl)piperidin-4-yl)propoxy)-2-methylphenyl)picolinic acid (35.4 mg, 35.93 umol, 60.86% yield, 97.45% purity) as an off-white solid.

MS (ESI) m/z: 960.6 [M+H]⁺.

¹H NMR (400 MHz, DMSO-d₆) δ=12.99-12.69 (m, 1H), 10.86 (s, 1H), 10.11-9.72 (m, 1H), 8.07-7.98 (m, 2H), 7.82-7.76 (m, 1H), 7.67-7.59 (m, 2H), 7.55-7.25 (m, 6H), 7.24-7.17 (m, 1H), 7.13-7.06 (m, 1H), 7.00-6.93 (m, 1H), 6.88 (d, J=8.4 Hz, 1H), 6.63 (d, J=8.0 Hz, 1H), 5.03-4.94 (m, 2H), 4.32 (dd, J=5.2, 9.2 Hz, 1H), 4.00-3.94 (m, 2H), 3.92 (s, 5H), 3.05-3.01 (m, 4H), 2.69-2.60 (m, 3H), 2.23-2.12 (m, 3H), 1.90 (s, 3H), 1.83-1.67 (m, 5H), 1.46-1.25 (m, 6H)

### Example 2. Preparation of Compound 108

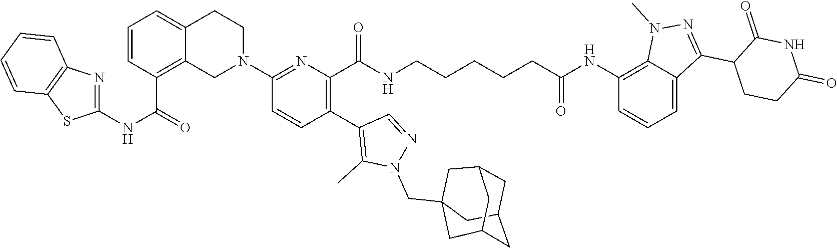 [SMILES: Cc1c(-c2ccc(N3CCc4cccc(C(=O)Nc5nc6ccccc6s5)c4C3)nc2C(=O)NCCCCCC(=O)Nc2cccc3c(C4CCC(=O)NC4=O)nn(C)c23)cnn1CC12CC3CC(CC(C3)C1)C2]

### 2-(5-(1-(((3r,5r,7r)-adamantan-1-yl)methyl)-5-methyl-1H-pyrazol-4-yl)-6-((6-((3-(2,6-dioxopiperidin-3-yl)-1-methyl-1H-indazol-7-yl)amino)-6-oxohexyl)carbamoyl)pyridin-2-yl)-N-(benzo[d]thiazol-2-yl)-1,2,3,4-tetrahydroisoquinoline-8-carboxamide

### Step A. Methyl 6-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)hexanoate

3-(1-(Adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinic acid (1 g, 1.52 mmol, 1 equiv.) and methyl 6-aminohexanoate (551.48 mg, 3.04 mmol, 2 equiv., HCl) were combined in DMF (10 mL). To this mixture was added HATU (692.58 mg, 1.82 mmol, 1.2 equiv.) and DIEA (588.53 mg, 4.55 mmol, 793.17 mL, 3 equiv.). The reaction mixture was stirred at 25° C. for 1 hour and then added dropwise to water 30 (mL), stirred briefly, and filtered to give a solid that was purified by silica gel chromatography (heptane/ethyl acetate gradient). Combined and concentrated product containing fractions to afford methyl 6-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)hexanoate (690 mg, 816.42 mmol, 53.79% yield).

MS (ESI) m/z: 786.9 [M+H]⁺.

### Step B. 6-(3-(1-(Adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)hexanoic acid

To a solution of methyl 6-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)hexanoate (670 mg, 852.42 mmol, 1 equiv.) in THE (7 mL) was added an aqueous solution of LiOH·H2O (1 M, 2.56 mL, 3 equiv.). The mixture was stirred at 25° C. for 12 hours. The pH of the reaction mixture was adjusted to 6 using an aqueous solution of HCl (1 M). The resulting solids were stirred briefly, filtered to give a crude product that was triturated with MeCN (5 mL) at 25° C. for 0.5 hour and then filtered to afford 6-(3-(1-(Adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)hexanoic acid (552.07 mg, 642.20 mmol, 75.34% yield).

MS (ESI) m/z: 772.1 [M+H]⁺; ¹H NMR (400 MHz, DMSO-d6) δ 8.10 (t, J=5.2 Hz, 1H), 7.90 (d, J=7.6 Hz, 1H), 7.66 (t, J=6.8 Hz, 2H), 7.45 (d, J=8.8 Hz, 1H), 7.41-7.26 (m, 4H), 7.26-7.21 (m, 2H), 6.93 (d, J=8.8 Hz, 1H), 5.05 (s, 2H), 3.89 (t, J=5.6 Hz, 2H), 3.04-2.98 (m, 4H), 2.10 (t, J=7.2 Hz, 2H), 2.05 (s, 3H), 1.92 (s, 3H), 1.68-1.61 (m, 4H), 1.58-1.51 (m, 10H), 1.41-1.36 (m, 2H), 1.33-1.27 (m, 2H), 1.15-1.08 (m, 2H).

### Step C. 2-(5-(1-(((3r,5r,7r)-adamantan-1-yl)methyl)-5-methyl-1H-pyrazol-4-yl)-6-((6-((3-(2,6-dioxopiperidin-3-yl)-1-methyl-1H-indazol-7-yl)amino)-6-oxohexyl)carbamoyl)pyridin-2-yl)-N-(benzo[d]thiazol-2-yl)-1,2,3,4-tetrahydroisoquinoline-8-carboxamide

To a solution of 6-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)hexanoic acid (80 mg, 103.63 mmol, 1 equiv.) and HATU (47.28 mg, 124.36 mmol, 1.2 equiv.) in DMF (1 mL) was added DIEA (40.18 mg, 310.89 mmol, 54.15 mL, 3 equiv.) and 3-(7-amino-1-methyl-1H-indazol-3-yl)piperidine-2,6-dione (29.44 mg, 113.99 mmol, 1.1 equiv.). The mixture was stirred at 25° C. for 12 hours, diluted with DMF (1.5 mL), and then purified by preparative RP HPLC (water/ACN, both with 0.1% FA modifier) to afford 2-(5-(1-(((3r,5r,7r)-adamantan-1-yl)methyl)-5-methyl-1H-pyrazol-4-yl)-6-((6-((3-(2,6-dioxopiperidin-3-yl)-1-methyl-1H-indazol-7-yl)amino)-6-oxohexyl)carbamoyl)pyridin-2-yl)-N-(benzo[d]thiazol-2-yl)-1,2,3,4-tetrahydroisoquinoline-8-carboxamide (24.94 mg, 23.73 mmol, 22.90% yield, 96.3% purity).

MS (ESI) m/z: 1013.2 [M+H]⁺;

¹H NMR (400 MHz, DMSO-d6) δ 12.94-12.76 (m, 1H), 10.89 (s, 1H), 9.78 (s, 1H), 8.10 (t, J=5.6 Hz, 1H), 8.02 (d, J=7.6 Hz, 1H), 7.79 (d, J=7.6 Hz, 1H), 7.59 (d, J=7.6 Hz, 2H), 7.50-7.43 (m, 2H), 7.42-7.38 (m, 1H), 7.37-7.30 (m, 2H), 7.25 (s, 1H), 7.11-7.02 (m, 2H), 6.95 (d, J=8.8 Hz, 1H), 4.99 (s, 2H), 4.44-4.30 (m, 1H), 4.00 (s, 3H), 3.89 (t, J=5.2 Hz, 2H), 3.68 (s, 2H), 3.09-2.97 (m, 4H), 2.65-2.61 (m, 1H), 2.32-2.30 (m, 1H), 2.20-2.15 (m, 1H), 2.06 (s, 3H), 1.91 (s, 4H), 1.68-1.58 (m, 4H), 1.58-1.48 (m, 12H), 1.40-1.34 (m, 2H), 1.22 (d, J=9.2 Hz, 2H).

### Example 3. Preparation of Compound 125a

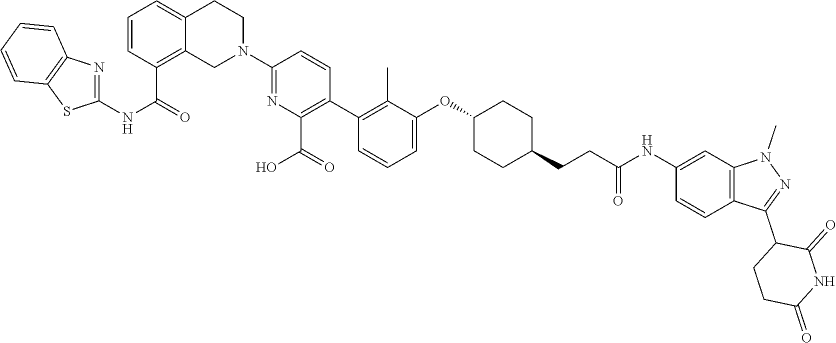 [SMILES: Cc1c(O[C@H]2CC[C@H](CCC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-(3-((3-(2,6-dioxopiperidin-3-yl)-1-methyl-1H-indazol-6-yl)amino)-3-oxopropyl)cyclohexyl)oxy)-2-methylphenyl)picolinic acid

### Step A. Ethyl (1r,4r)-4-(3-bromo-2-methylphenoxy)cyclohexane-1-carboxylate

To a solution of ethyl 4-hydroxycyclohexanecarboxylate (10 g, 58.06 mmol, 1 equiv.), 3-bromo-2-methyl-phenol (11.95 g, 63.87 mmol, 1.1 equiv.), PPh₃ (24.37 g, 92.90 mmol, 1.6 equiv.) in THE (100 mL) was added DIAD (18.79 g, 92.90 mmol, 18.06 mL, 1.6 equiv.) under 0° C. Then the mixture was stirred at 25° C. for 16 h. The reaction mixture was concentrated under reduced pressure to give a residue that was purified by silica gel chromatography (ethyl acetate/petroleum ether gradient) to afford ethyl (1r,4r)-4-(3-bromo-2-methylphenoxy)cyclohexane-1-carboxylate (3.6 g, 10.55 mmol, 18.17% yield). ¹H NMR (400 MHz, CDCl₃) δ 7.14 (d, J=8.0 Hz, 1H), 6.97 (t, J=8.0 Hz, 1H), 6.80 (d, J=8.0 Hz, 1H), 4.20-4.11 (m, 3H), 2.39-2.35 (m, 2H), 2.30 (s, 3H), 2.19-2.14 (m, 2H), 2.09-2.04 (m, 2H), 1.62-1.52 (m, 5H), 1.26 (t, J=6.8 Hz, 3H).

### Step B. ((1r,4r)-4-(3-Bromo-2-methylphenoxy)cyclohexyl)methanol

To a solution of ethyl 4-(3-bromo-2-methyl-phenoxy)cyclohexanecarboxylate (3.6 g, 10.55 mmol, 1 equiv.) in THE (15 mL) was added LiAlH₄ (480.49 mg, 12.66 mmol, 1.2 equiv.) under 0° C. The resulting mixture was then stirred at 25° C. for 1.5 h. The reaction was quenched by addition of a saturated aqueous solution of sodium sulfate (10 mL), and then extracted with ethyl acetate (10 mL×3). The combined organic layers were dried over anhydrous sodium sulfate, filtered, and concentrated under reduced pressure to afford ((1r,4r)-4-(3-Bromo-2-methylphenoxy)cyclohexyl)methanol (2.96 g, crude) that was used without further purification.

¹H NMR (400 MHz, CDCl₃) δ 7.13 (d, J=8.0 Hz, 1H), 6.97 (t, J=8.0 Hz, 1H), 6.81 (d, J=8.4 Hz, 1H), 4.13-4.08 (m, 1H), 3.50 (d, J=6.4 Hz, 2H), 2.30 (s, 3H), 2.20-2.14 (m, 2H), 1.95-1.86 (m, 2H), 1.53-1.45 (m, 3H), 1.14-1.04 (m, 2H).

### Step C. (1r,4r)-4-(3-Bromo-2-methylphenoxy)cyclohexane-1-carbaldehyde

To a solution of DMSO (3.09 g, 39.57 mmol, 3.09 mL, 4 equiv.) in DCM (10 mL) was added dropwise a solution of oxalyl chloride (2.51 g, 19.79 mmol, 1.73 mL, 2 equiv.) in DCM (2 mL) at −70° C. under a nitrogen atmosphere. The mixture was stirred at −70° C. for 1 h. ((1r,4r)-4-(3-Bromo-2-methylphenoxy)cyclohexyl)methanol (2.96 g, 9.89 mmol, 1 equiv.) in DCM (10 mL) was then added dropwise at −70° C. The solution was stirred for 1 h at −70° C. TEA (6.01 g, 59.36 mmol, 8.26 mL, 6 equiv.) was added, and the mixture was stirred at −70° C. for 0.5 h under a nitrogen atmosphere. The reaction mixture was quenched by addition water (10 mL), diluted with DCM (10 mL), and extracted with DCM (10 mL×3). The combined organic layers were washed with water (5 mL×3), dried over anhydrous sodium sulfate, filtered, and concentrated under reduced pressure to afford (1r,4r)-4-(3-bromo-2-methylphenoxy)cyclohexane-1-carbaldehyde (3 g, crude) that was used without further purification.

¹H NMR (400 MHz, DMSO-d6) δ 9.62-9.59 (m, 1H), 7.16-7.01 (m, 3H), 4.36-4.23 (m, 1H), 2.39-2.31 (m, 1H), 2.25-2.18 (m, 3H), 2.03-1.92 (m, 4H), 1.50-1.38 (m, 4H).

### Step D. Ethyl (E)-3-((1r,4r)-4-(3-bromo-2-methylphenoxy)cyclohexyl)acrylate

To a solution of NaH (56.52 mg, 1.41 mmol, 60% purity, 2.1 equiv.) in THE (3 mL) at 0° C. was added ethyl 2-diethoxyphosphorylacetate (301.75 mg, 1.35 mmol, 267.04 mL, 2 equiv.). (1r,4r)-4-(3-Bromo-2-methylphenoxy)cyclohexane-1-carbaldehyde (200 mg, 672.98 mmol, 1 equiv.) was then added, and the mixture was stirred at 25° C. for 6 h. The reaction mixture was quenched by addition of a saturated aqueous solution of ammonium chloride (5 mL) at 0° C., and then extracted with ethyl acetate (10 mL×3). The combined organic layers were washed with water (10 mL), dried over anhydrous sodium sulfate, filtered, and concentrated under reduced pressure to give a residue which was purified by silica gel chromatography (ethyl acetate/petroleum ether gradient) to afford the title compound (140 mg, 362.12 mmol, 53.81% yield).

¹H NMR (400 MHz, CDCl₃) δ 7.14 (d, J=8.0 Hz, 1H), 7.01-6.87 (m, 2H), 6.80 (d, J=8.4 Hz, 1H), 5.82 (dd, J=1.2, 15.6 Hz, 1H), 4.21-4.15 (m, 2H), 4.16-4.07 (m, 1H), 2.30 (s, 3H), 2.26-2.16 (m, 3H), 1.97-1.89 (m, 2H), 1.58-1.48 (m, 2H), 1.33-1.26 (m, 5H).

### Step E. Ethyl 3-((1r,4r)-4-(3-bromo-2-methylphenoxy)cyclohexyl)propanoate

A mixture of ethyl (E)-3-[4-(3-bromo-2-methyl-phenoxy)cyclohexyl]prop-2-enoate (1.6 g, 4.36 mmol, 1 equiv.) and PtO₂ (98.92 mg, 435.64 mmol, 0.1 equiv.) in EtOH (15 mL) was degassed and purged with hydrogen three times. The mixture was stirred at 25° C. for 3 hours under a hydrogen atmosphere (balloon, ˜15 psi). The reaction mixture was filtered and concentrated under reduced pressure to give a residue that was purified by preparative RP-HPLC (water/acetonitrile with 0.1% formic acid in each). Combined and lyophilized pure product containing fractions to afford ethyl 3-((1r,4r)-4-(3-bromo-2-methylphenoxy)cyclohexyl)propanoate (1.4 g, 3.79 mmol, 87.02% yield).

¹H NMR (400 MHz, DMSO-d6) δ 7.17-7.10 (m, 1H), 7.06 (t, J=8.0 Hz, 1H), 7.03-6.99 (m, 1H), 4.27-4.17 (m, 1H), 4.11-4.04 (m, 2H), 2.33-2.26 (m, 2H), 2.20 (s, 3H), 2.03 (d, J=10.0 Hz, 2H), 1.75 (d, J=12.0 Hz, 2H), 1.55-1.42 (m, 2H), 1.39-1.21 (m, 3H), 1.17 (t, J=7.2 Hz, 3H), 1.11-0.97 (m, 2H).

### Step F. tert-Butyl 6-(8-(benzo[d]thiazol-2-ylcarbamoyl)-3,4-dihydroisoquinolin-2(1H)-yl)-3-(3-(((1r,4r)-4-(3-ethoxy-3-oxopropyl)cyclohexyl)oxy)-2-methylphenyl)picolinate

A mixture of ethyl 3-((1r,4r)-4-(3-bromo-2-methylphenoxy)cyclohexyl)propanoate (200 mg, 541.58 mmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (546.40 mg, 758.21 mmol, 1.4 equiv.), [2-(2-aminophenyl)phenyl]palladium(1+); bis(1-adamantyl)-butyl-phosphane; methanesulfonate (78.88 mg, 108.32 mmol, 0.2 equiv.), and an aqueous solution of potassium carbonate (1.5 M, 541.58 mL, 1.5 equiv.) in dioxane (2.5 mL) was degassed and purged with nitrogen three times. The mixture was stirred at 100° C. for 1 h under microwave heating. The reaction mixture was filtered and concentrated under reduced pressure to provide a residue that was purified by silica gel chromatography (ethyl acetate/petroleum ether gradient) to afford tert-butyl 6-(8-(benzo[d]thiazol-2-ylcarbamoyl)-3,4-dihydroisoquinolin-2(1H)-yl)-3-(3-(((1r,4r)-4-(3-ethoxy-3-oxopropyl)cyclohexyl)oxy)-2-methylphenyl)picolinate (440 mg, crude).

¹H NMR (400 MHz, DMSO-d6) δ 8.02 (d, J=7.6 Hz, 1H), 7.78 (d, J=8.0 Hz, 1H), 7.60 (d, J=7.5 Hz, 1H), 7.50-7.40 (m, 3H), 7.40-7.30 (m, 2H), 7.12-7.03 (m, 1H), 6.99-6.89 (m, 2H), 6.59-6.49 (m, 1H), 5.03-4.90 (m, 2H), 4.27-4.14 (m, 1H), 4.03 (d, J=6.8 Hz, 2H), 3.87 (t, J=5.6 Hz, 2H), 3.29 (s, 2H), 3.03 (t, J=5.6 Hz, 2H), 2.29 (t, J=7.6 Hz, 2H), 2.05 (d, J=11.2 Hz, 1H), 1.84 (s, 3H), 1.79-1.70 (m, 2H), 1.50-1.42 (m, 2H), 1.23 (d, J=8.4 Hz, 2H), 1.19-1.17 (m, 3H), 1.07 (s, 2H), 1.00 (s, 9H).

### Step G. 3-((1r,4r)-4-(3-(6-(8-(Benzo[d]thiazol-2-ylcarbamoyl)-3,4-dihydroisoquinolin-2(1H)-yl)-2-(tert-butoxycarbonyl)pyridin-3-yl)-2-methylphenoxy)cyclohexyl)propanoic acid

A mixture of tert-butyl 6-(8-(benzo[d]thiazol-2-ylcarbamoyl)-3,4-dihydroisoquinolin-2(1H)-yl)-3-(3-(((1r,4r)-4-(3-ethoxy-3-oxopropyl)cyclohexyl)oxy)-2-methylphenyl)picolinate (440 mg, 567.77 mmol, 1 equiv.) and LiOH monohydrate (71.47 mg, 1.70 mmol, 3 equiv.) in THE (1.2 mL) and water (0.4 mL) was stirred at 25° C. for 1.5 h. The mixture was concentrated and redissolved in water (20 mL), and an aqueous solution of 1M HCl was added to adjust the pH to 2. The resulting solids were filtered and dried under reduced pressure to afford 3-((1r,4r)-4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)cyclohexyl)propanoic acid (380 mg, 508.76 mmol, 89.61% yield).

### Step H. tert-butyl 6-(8-(benzo[d]thiazol-2-ylcarbamoyl)-3,4-dihydroisoquinolin-2(1H)-yl)-3-(3-(((1r,4r)-4-(3-((3-(2,6-dioxopiperidin-3-yl)-1-methyl-1H-indazol-6-yl)amino)-3-oxopropyl)cyclohexyl)oxy)-2-methylphenyl)picolinate

A mixture of 3-((1r,4r)-4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)cyclohexyl)propanoic acid (120 mg, 160.66 μmol, 1 equiv.), 3-(6-amino-1-methyl-indazol-3-yl)piperidine-2,6-dione (45.64 mg, 176.73 μmol, 1.1 equiv.), HATU (73.31 mg, 192.79 μmol, 1.2 equiv.), and N,N-diisopropylethylamine (62.29 mg, 481.98 μmol, 83.95 uL, 3 equiv.) in DMF (0.5 mL) was stirred at 25° C. for 12 hours. The reaction mixture was diluted with water (1 mL) and extracted with ethyl acetate (1 mL×3). The combined organic layers were washed with water, dried over anhydrous sodium sulfate, and concentrated under reduced pressure to give crude tert-butyl 6-(8-(benzo[d]thiazol-2-ylcarbamoyl)-3,4-dihydroisoquinolin-2(1H)-yl)-3-(3-(((1r,4r)-4-(3-((3-(2,6-dioxopiperidin-3-yl)-1-methyl-1H-indazol-6-yl)amino)-3-oxopropyl)cyclohexyl)oxy)-2-methylphenyl)picolinate (120 mg) as a white solid which was carried forward without further purification.

Step I. 6-(8-(benzo[d]thiazol-2-ylcarbamoyl)-3,4-dihydroisoquinolin-2(1H)-yl)-3-(3-(((1r,4r)-4-(3-((3-(2,6-dioxopiperidin-3-yl)-1-methyl-1H-indazol-6-yl)amino)-3-oxopropyl)cyclohexyl)oxy)-2-methylphenyl)picolinic acid

tert-Butyl 6-(8-(benzo[d]thiazol-2-ylcarbamoyl)-3,4-dihydroisoquinolin-2(1H)-yl)-3-(3-(((1r,4r)-4-(3-((3-(2,6-dioxopiperidin-3-yl)-1-methyl-1H-indazol-6-yl)amino)-3-oxopropyl)cyclohexyl)oxy)-2-methylphenyl)picolinate (120 mg, 121.56 μmol, 1 equiv.) in trifluoroacetic acid (0.5 mL) and DCM (0.5 mL) was stirred at 40° C. for 10 hours. The reaction mixture was concentrated under reduced pressure. The resulting crude material was purified by prep-HPLC (column: Phenomenex luna C18 150×25 mm×10 um; mobile phase: [water (1% formic acid)-acetonitrile]; B %: 56%-86%, 10 min) to give 6-(8-(benzo[d]thiazol-2-ylcarbamoyl)-3,4-dihydroisoquinolin-2(1H)-yl)-3-(3-(((1r,4r)-4-(3-((3-(2,6-dioxopiperidin-3-yl)-1-methyl-1H-indazol-6-yl)amino)-3-oxopropyl)cyclohexyl)oxy)-2-methylphenyl)picolinic acid (35.76 mg, 37.68 μmol, 31% yield, 98.1% purity) as a white solid.

MS (ESI) m/z: 932.3 [M+H]⁺.

¹H NMR (400 MHz, DMSO-d6) δ 12.94-12.74 (m, 1H), 10.89-10.80 (m, 1H), 10.05 (s, 1H), 8.09-8.00 (m, 2H), 7.82-7.75 (m, 1H), 7.65-7.59 (m, 2H), 7.53-7.31 (m, 6H), 7.12-7.05 (m, 2H), 7.00-6.90 (m, 2H), 6.61 (d, J=7.6 Hz, 1H), 4.98 (s, 2H), 4.34-4.28 (m, 1H), 4.26-4.17 (m, 1H), 3.91 (d, J=2.0 Hz, 5H), 3.03 (s, 2H), 2.67-2.61 (m, 2H), 2.42-2.30 (m, 5H), 2.20-2.07 (m, 4H), 1.89-1.80 (m, 5H), 1.57 (d, J=6.0 Hz, 2H), 1.39-1.32 (m, 2H).

### Example 4. Preparation of Compound 130

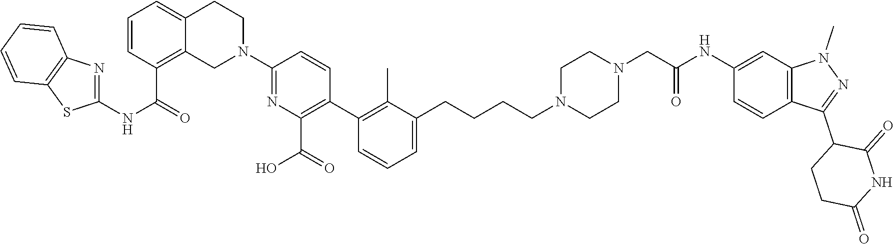 [SMILES: Cc1c(CCCCN2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-[8-(1,3-Benzothiazol-2-ylcarbamoyl)-3,4-dihydro-1H-isoquinolin-2-yl]-3-[3-[4-[4-[2-[[3-(2,6-dioxo-3-piperidyl)-1-methyl-indazol-6-yl]amino]-2-oxo-ethyl]piperazin-1-yl]butyl]-2-methyl-phenyl]pyridine-2-carboxylic acid

### Step A. 4-(3-bromo-2-methyl-phenyl)but-3-yn-1-ol

A mixture of 1-bromo-3-iodo-2-methyl-benzene (5 g, 16.84 mmol, 1 equiv.), but-3-yn-1-ol (1.77 g, 25.26 mmol, 1.91 mL, 1.5 equiv.), Pd(PPh₃)₄ (778.34 mg, 673.56 μmol, 0.04 equiv.), CuI (128.28 mg, 673.56 μmol, 0.04 equiv.) and in TEA (40 mL) was degassed and purged with nitrogen three times, and then the mixture was stirred at 90° C. for 16 hours under a nitrogen atmosphere. The reaction mixture was concentrated under reduced pressure to remove solvent to give a residue which was purified by silica gel chromatography (petroleum ether/ethyl acetate=5/1 to 4/1) to afford 4-(3-bromo-2-methyl-phenyl)but-3-yn-1-ol (3.6 g, 15.06 mmol, 89.4% yield).

¹H NMR (400 MHz, CDCl₃) δ=7.39 (d, J=8.0 Hz, 1H), 7.26 (d, J=7.6 Hz, 1H), 6.88 (t, J=7.6 Hz, 1H), 3.76 (t, J=6.4 Hz, 2H), 2.66 (t, J=6.4 Hz, 2H), 2.45 (s, 3H), 1.79 (s, 1H).

### Step B. 4-(3-Bromo-2-methyl-phenyl)butan-1-ol

A mixture of 4-(3-bromo-2-methyl-phenyl)but-3-yn-1-ol (3.5 g, 14.64 mmol, 1 equiv.), PtO₂ (332.39 mg, 1.46 mmol, 0.1 equiv.) in EtOH (5 mL) was degassed and purged with hydrogen gas three times, and then the mixture was stirred at 30° C. for 5 hours under a hydrogen atmosphere. The reaction mixture was filtered and concentrated under reduced pressure to give 4-(3-bromo-2-methyl-phenyl)butan-1-ol that was used without further purification assuming theoretical yield (4 g, crude).

¹H NMR (400 MHz, CDCl₃) δ=7.32 (d, J=8.0 Hz, 1H), 6.99 (d, J=7.6 Hz, 1H), 6.91-6.85 (m, 1H), 3.67-3.57 (m, 3H), 2.64-2.58 (m, 2H), 2.31 (s, 3H), 1.29 (s, 2H), 1.16 (t, J=7.2 Hz, 2H).

### Step C. 1-Bromo-3-(4-bromobutyl)-2-methyl-benzene

To a solution of 4-(3-bromo-2-methyl-phenyl)butan-1-ol (2.0 g, 8.23 mmol, 1 equiv.) in DCM (10 mL) was added PPh₃ (2.37 g, 9.05 mmol, 1.1 equiv.) and CBr₄ (4.09 g, 12.34 mmol, 1.5 equiv.). The mixture was stirred at 25° C. for 16 hours. The reaction mixture was concentrated under reduced pressure to remove solvent to give a residue which was purified by silica gel chromatography (petroleum ether/ethyl acetate=20/1 to 10/1) to afford 1-bromo-3-(4-bromobutyl)-2-methyl-benzene (1.6 g, 5.23 mmol, 63.6% yield).

¹H NMR (400 MHz, CDCl₃) δ=7.34 (d, J=8.0 Hz, 1H), 6.99 (d, J=7.6 Hz, 1H), 6.92-6.86 (m, 1H), 3.36 (t, J=6.8 Hz, 2H), 2.64-2.58 (m, 2H), 2.32 (s, 3H), 1.90-1.81 (m, 2H), 1.69-1.59 (m, 2H).

### Step D. Ethyl 2-[4-[4-(3-bromo-2-methyl-phenyl)butyl]piperazin-1-yl]acetate

To a solution of 1-bromo-3-(4-bromobutyl)-2-methyl-benzene (1.6 g, 5.23 mmol, 1 equiv.) in MeCN (20 mL) was added ethyl 2-piperazin-1-ylacetate (900.41 mg, 5.23 mmol, 1 equiv.) and K₂CO₃ (2.17 g, 15.68 mmol, 3 equiv.). The mixture was stirred at 60° C. for 16 hours. The reaction mixture was filtered and concentrated under reduced pressure to remove solvent to give a residue which was purified by silica gel chromatography (DCM:MeOH=1/0 to 10:1) to afford ethyl 2-[4-[4-(3-bromo-2-methyl-phenyl)butyl]piperazin-1-yl]acetate (1.6 g, 4.03 mmol, 77.0% yield).

MS (ESI) m/z: 398.9 [M+H]⁺.

### Step E. tert-Butyl 6-[8-(1,3-benzothiazol-2-ylcarbamoyl)-3,4-dihydro-1H-isoquinolin-2-yl]-3-[3-[4-[4-(2-ethoxy-2-oxo-ethyl)piperazin-1-yl]butyl]-2-methyl-phenyl]pyridine-2-carboxylate

A mixture of ethyl 2-[4-[4-(3-bromo-2-methyl-phenyl)butyl]piperazin-1-yl]acetate (300 mg, 0.755 mmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (462.47 mg, 0.755 mmol, 1 equiv.), K₂CO₃ (313.04 mg, 2.27 mmol, 3 equiv.), and Ad₂ⁿBuP Pd G3 (cataCXium® A Pd G3) (54.98 mg, 0.076 mmol, 0.1 equiv.) in dioxane (5 mL) and H₂O (2 mL) was degassed and purged with nitrogen gas three times. The mixture was stirred at 100° C. for 2 hours under a nitrogen atmosphere. The reaction mixture was concentrated under reduced pressure to provide a residue which was purified by silica gel chromatography (DCM:MeOH=1/0 to 10:1) to afford tert-butyl 6-[8-(1,3-benzothiazol-2-ylcarbamoyl)-3,4-dihydro-1H-isoquinolin-2-yl]-3-[3-[4-[4-(2-ethoxy-2-oxo-ethyl)piperazin-1-yl]butyl]-2-methyl-phenyl]pyridine-2-carboxylate (280 mg, 0.035 mmol, 46.2% yield) as a yellow oil.

MS (ESI) m/z: 803.5 [M+H]⁺.

### Step F. 2-[4-[4-[3-[6-[8-(1,3-Benzothiazol-2-ylcarbamoyl)-3,4-dihydro-1H-isoquinolin-2-yl]-2-tert-butoxycarbonyl-3-pyridyl]-2-methyl-phenyl]butyl]piperazin-1-yl]acetic acid

To a solution of tert-butyl 6-[8-(1,3-benzothiazol-2-ylcarbamoyl)-3,4-dihydro-1H-isoquinolin-2-yl]-3-[3-[4-[4-(2-ethoxy-2-oxo-ethyl)piperazin-1-yl]butyl]-2-methyl-phenyl]pyridine-2-carboxylate (250 mg, 0.31 mmol, 1 equiv.) in THE (3 mL) and H₂O (1 mL) was added LiOH·H₂O (78.39 mg, 1.87 mmol, 6.0 equiv.). The mixture was stirred at 25° C. for 12 hours. The reaction mixture was concentrated under reduced pressure to give a residue that was diluted with water (10 mL) and extracted with EtOAc (3×10 mL). The combined organic layers were concentrated under reduced pressure to afford 2-[4-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenyl]butyl]piperazin-1-yl]acetic acid which was used without further purification (200 mg, 0.26 mmol, 82.9% yield).

MS (ESI) m/z: 775.5 [M+H]⁺.

### Step G. tert-Butyl 6-[8-(1,3-benzothiazol-2-ylcarbamoyl)-3,4-dihydro-1H-isoquinolin-2-yl]-3-[3-[4-[4-[2-[[3-(2,6-dioxo-3-piperidyl)-1-methyl-indazol-6-yl]amino]-2-oxo-ethyl]piperazin-1-yl]butyl]-2-methyl-phenyl]pyridine-2-carboxylate

To a solution of 2-[4-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenyl]butyl]piperazin-1-yl]acetic acid (78 mg, 0.10 mmol, 1 equiv.) in DMF (2 mL) was added HATU (38.27 mg, 0.10 mmol, 1 equiv.), 3-(6-amino-1-methyl-indazol-3-yl)piperidine-2,6-dione (26.00 mg, 0.10 mmol, 1 equiv.), and DIEA (39 mg, 0.30 mmol, 1 equiv.). The mixture was stirred at 25° C. for 2 hours, then diluted with water (10 mL), filtered, and concentrated under reduced pressure to afford tert-butyl 6-[8-(1,3-benzothiazol-2-ylcarbamoyl)-3,4-dihydro-1H-isoquinolin-2-yl]-3-[3-[4-[4-[2-[[3-(2,6-dioxo-3-piperidyl)-1-methyl-indazol-6-yl]amino]-2-oxo-ethyl]piperazin-1-yl]butyl]-2-methyl-phenyl]pyridine-2-carboxylate that was used without further purification (100 mg, 0.098 mmol, 97.9% yield).

MS (ESI) m/z: 507.5 [M+2H]/2⁺.

### Step H. 6-[8-(1,3-Benzothiazol-2-ylcarbamoyl)-3,4-dihydro-1H-isoquinolin-2-yl]-3-[3-[4-[4-[2-[[3-(2,6-dioxo-3-piperidyl)-1-methyl-indazol-6-yl]amino]-2-oxo-ethyl]piperazin-1-yl]butyl]-2-methyl-phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[4-[4-[2-[[3-(2,6-dioxo-3-piperidyl)-1-methyl-indazol-6-yl]amino]-2-oxo-ethyl]piperazin-1-yl]butyl]-2-methyl-phenyl]pyridine-2-carboxylate (70 mg, 0.069 mmol, 1 equiv.) in TFA (1 mL) and DCM (1 mL) was stirred at 25° C. for 12 hours. The reaction mixture was concentrated under reduced pressure, and the resulting residue was purified by preparative RP HPLC (water/MeCN, both with 0.1% formic acid modifier) to afford 6-[8-(1,3-benzothiazol-2-ylcarbamoyl)-3,4-dihydro-1H-isoquinolin-2-yl]-3-[3-[4-[4-[2-[[3-(2,6-dioxo-3-piperidyl)-1-methyl-indazol-6-yl]amino]-2-oxo-ethyl]piperazin-1-yl]butyl]-2-methyl-phenyl]pyridine-2-carboxylic acid (12.87 mg, 0.013 mmol, 19.3% yield, 99.4% purity).

MS (ESI) m/z: 959.5 [M+H]⁺;

¹H NMR (400 MHz, DMSO-d6) δ=10.87 (s, 1H), 9.85 (s, 1H), 8.24 (s, 1H), 8.07-7.97 (m, 2H), 7.78 (d, J=8.4 Hz, 1H), 7.63 (d, J=9.2 Hz, 2H), 7.46-7.41 (m, 2H), 7.39-7.33 (m, 2H), 7.20 (d, J=8.8 Hz, 1H), 7.10-7.02 (m, 2H), 6.90 (t, J=9.2 Hz, 2H), 4.99 (s, 2H), 4.33 (dd, J=4.8, 9.4 Hz, 1H), 3.93 (s, 5H), 3.15 (s, 3H), 3.02 (d, J=5.6 Hz, 2H), 2.69-2.58 (m, 6H), 2.41-2.39 (m, 2H), 2.34 (s, 4H), 2.24 (s, 1H), 2.02 (s, 3H), 1.56-1.46 (m, 4H).

### Example 5. Preparation of Compound 133

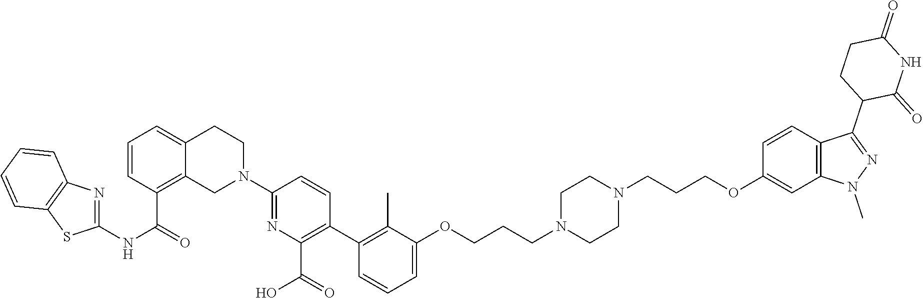 [SMILES: Cc1c(OCCCN2CCN(CCCOc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-(8-(Benzo[d]thiazol-2-ylcarbamoyl)-3,4-dihydroisoquinolin-2(1H)-yl)-3-(3-(3-(4-(3-((3-(2,6-dioxopiperidin-3-yl)-1-methyl-1H-indazol-6-yl)oxy)propyl)piperazin-1-yl)propoxy)-2-methylphenyl)picolinic acid

### Step A. (3-(3-Bromo-2-methylphenoxy)propoxy)(tert-butyl)dimethylsilane

3-Bromo-2-methylphenol (2.00 g, 1 equiv., 10.7 mmol), (3-bromopropoxy)(tert-butyl)dimethylsilane (3.39 g, 1.25 equiv., 13.4 mmol), and potassium carbonate (2.22 g, 1.50 equiv., 16.0 mmol) were combined in DMF and heated overnight at 50° C. Diluted the reaction mixture with ethyl acetate (100 mL) and brine (25 mL). Separated layers, dried organic layer over anhydrous magnesium sulfate, filtered, and concentrated to an oil that was purified by silica gel chromatography (heptane/ethyl acetate gradient). Combined and concentrated product containing fractions to afford (3-(3-bromo-2-methylphenoxy)propoxy)(tert-butyl)dimethylsilane (3.49 g, 9.71 mmol, 90.8% yield) returned.

MS (ESI) m/z: 359.2 [M+H]⁺.

### Step B. tert-Butyldimethyl(3-(2-methyl-3-(4,4,5,5-tetramethyl-1,3,2-dioxaborolan-2-yl)phenoxy)propoxy)silane

Combined (3-(3-bromo-2-methylphenoxy)propoxy)(tert-butyl)dimethylsilane (1.00 g, 1 equiv., 2.78 mmol), bis(pinacolato)diboron (1.20 g, 1.7 equiv., 4.73 mmol), and PdCl₂(dppf)-CH₂Cl₂ adduct (227 mg, 0.1 equiv., 278 μmol) in 1,4-dioxane (15 mL). Added potassium acetate (819 mg, 3 equiv., 8.35 mmol) and let mixture stir at 90° C. overnight. Diluted reaction mixture with 125 mL of ethyl acetate, 25 mL of water, and 25 mL of brine. Separated layers and washed organic layer with an additional 50 mL of brine, dried over anhydrous magnesium sulfate, filtered, and concentrated to dryness under reduced pressure. Purified residue by silica gel chromatography (heptane/ethyl acetate gradient). Combined and concentrated product containing fractions to afford tert-butyldimethyl(3-(2-methyl-3-(4,4,5,5-tetramethyl-1,3,2-dioxaborolan-2-yl)phenoxy)propoxy)silane (0.862 g, 2.12 mmol, 76.2% yield).

MS (ESI) m/z: 407. [M+H]⁺.

### Step C. tert-Butyl 6-(8-(benzo[d]thiazol-2-ylcarbamoyl)-3,4-dihydroisoquinolin-2(1H)-yl)-3-(3-(3-((tert-butyldimethylsilyl)oxy)propoxy)-2-methylphenyl)picolinate

tert-Butyl 6-(8-(benzo[d]thiazol-2-ylcarbamoyl)-3,4-dihydroisoquinolin-2(1H)-yl)-3-bromopicolinate (662 mg, 1 equiv., 1.17 mmol) and tert-butyldimethyl(3-(2-methyl-3-(4,4,5,5-tetramethyl-1,3,2-dioxaborolan-2-yl)phenoxy)propoxy)silane (571 mg, 1.2 equiv., 1.40 mmol) from were dissolved in 1,4-dioxane (8.0 mL). Added potassium phosphate (745 mg, 2.34 mL, 1.5 molar, 3 equiv., 3.51 mmol) and CataCxium Pd G3 (42.6 mg, 0.05 equiv., 58.5 μmol). Let mixture stir at 100° C. for 40 minutes under microwave heating. Diluted reaction mixture with 125 mL of ethyl acetate, 25 mL of water, and 25 mL of brine. Separated layers and washed organic layer with an additional 50 mL of brine, dried over anhydrous magnesium sulfate, filtered, and concentrated to dryness under reduced pressure. Purified residue by silica gel chromatography (heptane/ethyl acetate gradient). Combined and concentrated product containing fractions to afford tert-butyl 6-(8-(benzo[d]thiazol-2-ylcarbamoyl)-3,4-dihydroisoquinolin-2(1H)-yl)-3-(3-(3-((tert-butyldimethylsilyl)oxy)propoxy)-2-methylphenyl)picolinate (530 mg, 693 μmol, 59.2% yield) returned.

MS (ESI) m/z: 765.3 [M+H]⁺.

### Step D. tert-Butyl 6-(8-(benzo[d]thiazol-2-ylcarbamoyl)-3,4-dihydroisoquinolin-2(1H)-yl)-3-(3-(3-hydroxypropoxy)-2-methylphenyl)picolinate

TBAF (217 mg, 831 μL, 1.0 molar, 1.2 equiv., 831 μmol), as a 1N solution in THF, and tert-butyl 6-(8-(benzo[d]thiazol-2-ylcarbamoyl)-3,4-dihydroisoquinolin-2(1H)-yl)-3-(3-(3-((tert-butyldimethylsilyl)oxy)propoxy)-2-methylphenyl)picolinate (530 mg, 1 equiv., 693 μmol) were combined in THF (10.0 mL) and mixed at ambient temperature overnight. Added an additional portion of TBAF (217 mg, 831 μL, 1.0 molar, 1.2 equiv., 831 μmol) and let mix again overnight at ambient temperature. Diluted reaction mixture with 125 mL of ethyl acetate, 25 mL of water, and 25 mL of brine. Separated layers and washed organic layer with an additional 50 mL of brine, dried over anhydrous magnesium sulfate, filtered, and concentrated to dryness under reduced pressure. Purified residue by silica gel chromatography (heptane/ethyl acetate gradient). Combined and concentrated product containing fractions to afford tert-butyl 6-(8-(benzo[d]thiazol-2-ylcarbamoyl)-3,4-dihydroisoquinolin-2(1H)-yl)-3-(3-(3-hydroxypropoxy)-2-methylphenyl)picolinate (322 mg, 495 μmol, 71.4% yield).

MS (ESI) m/z: 651.2 [M+H]⁺.

### Step E. tert-butyl 6-(8-(benzo[d]thiazol-2-ylcarbamoyl)-3,4-dihydroisoquinolin-2(1H)-yl)-3-(3-(3-chloropropoxy)-2-methylphenyl)picolinate

tert-Butyl 6-(8-(benzo[d]thiazol-2-ylcarbamoyl)-3,4-dihydroisoquinolin-2(1H)-yl)-3-(3-(3-hydroxypropoxy)-2-methylphenyl)picolinate (322 mg, 1 equiv., 495 μmol) was dissolved in DCM (10 mL). Added thionyl chloride (118 mg, 72.2 μL, 2 equiv., 990 μmol) and let reaction mix overnight at ambient temperature. Diluted reaction mixture with additional DCM and a saturated aqueous solution of sodium bicarbonate. Separated layers, dried organic layer over anhydrous magnesium sulfate, filtered, and concentrated to dryness under vacuum. Purified residue by silica gel chromatography (heptane/ethyl acetate gradient). Combined and concentrated product containing fractions to afford tert-butyl 6-(8-(benzo[d]thiazol-2-ylcarbamoyl)-3,4-dihydroisoquinolin-2(1H)-yl)-3-(3-(3-chloropropoxy)-2-methylphenyl)picolinate (163 mg, 244 μmol, 49.2% yield).

MS (ESI) m/z: 669.1 [M+H]⁺.

### Step F. tert-Butyl 4-(3-((3-(2,6-bis(benzyloxy)pyridin-3-yl)-1-methyl-1H-indazol-6-yl)oxy)propyl)piperazine-1-carboxylate

tert-Butyl 4-(3-hydroxypropyl)piperazine-1-carboxylate (195 mg, 2 equiv., 799 μmol) and 3-(2,6-bis(benzyloxy)pyridin-3-yl)-6-bromo-1-methyl-1H-indazole (200 mg, 1 equiv., 400 μmol) in dioxane (5 mL). Added tBuBrettPhos Pd G3 (17.1 mg, 0.05 equiv., 20.0 μmol) followed by potassium tert-butoxide (53.8 mg, 480 μL, 1.0 molar, 1.2 equiv., 480 μmol) and let mix at ambient temperature for 48 h. Diluted reaction mixture with 125 mL of ethyl acetate, 25 mL of water, and 25 mL of brine. Separated layers and washed organic layer with an additional 50 mL of brine, dried over anhydrous magnesium sulfate, filtered, and concentrated to dryness under reduced pressure. Purified residue by silica gel chromatography (heptane/ethyl acetate containing 10% of a 7N solution of NH₃ in MeOH). Concentrated product containing fractions to afford tert-butyl 4-(3-((3-(2,6-bis(benzyloxy)pyridin-3-yl)-1-methyl-1H-indazol-6-yl)oxy)propyl)piperazine-1-carboxylate (170 mg, 256 μmol, 64.1% yield).

MS (ESI) m/z: 664.3 [M+H]⁺.

### Step G. tert-Butyl 4-(3-((3-(2,6-dioxopiperidin-3-yl)-1-methyl-1H-indazol-6-yl)oxy)propyl)piperazine-1-carboxylate

Dissolved tert-butyl 4-(3-((3-(2,6-bis(benzyloxy)pyridin-3-yl)-1-methyl-1H-indazol-6-yl)oxy)propyl)piperazine-1-carboxylate (170 mg, 1 equiv., 256 μmol) in Ethyl acetate (10 mL). Added palladium on carbon (545 mg, 5% wt, 1 equiv., 256 μmol) and degassed reaction under vacuum. Let mix at ambient temperature under an hydrogen atmosphere for 48 h. Filtered reaction through a pad of Celite, rinsed Celite with ethyl acetate, and concentrated reaction to afford tert-butyl 4-(3-((3-(2,6-dioxopiperidin-3-yl)-1-methyl-1H-indazol-6-yl)oxy)propyl)piperazine-1-carboxylate (79.6 mg, 164 μmol, 64.0% yield).

MS (ESI) m/z: 486.3 [M+H]⁺.

### Step H. 3-(1-Methyl-6-(3-(piperazin-1-yl)propoxy)-1H-indazol-3-yl)piperidine-2,6-dione, 2HCl

Dissolved tert-butyl 4-(3-((3-(2,6-dioxopiperidin-3-yl)-1-methyl-1H-indazol-6-yl)oxy)propyl)piperazine-1-carboxylate (79.6 mg, 1 equiv., 164 μmol) in DCM (10 mL). Added HCl in dioxane (239 mg, 1.64 mL, 4 molar, 40.0 equiv., 6.56 mmol) and let mix under ambient temperature overnight. Concentrated material to afford 3-(1-methyl-6-(3-(piperazin-1-yl)propoxy)-1H-indazol-3-yl)piperidine-2,6-dione, 2HCl (85 mg, 0.19 mmol).

MS (ESI) m/z: 386.3 [M+H]⁺.

### Step I. tert-Butyl 6-(8-(benzo[d]thiazol-2-ylcarbamoyl)-3,4-dihydroisoquinolin-2(1H)-yl)-3-(3-(3-(4-(3-((3-(2,6-dioxopiperidin-3-yl)-1-methyl-1H-indazol-6-yl)oxy)propyl)piperazin-1-yl)propoxy)-2-methylphenyl)picolinate

3-(1-Methyl-6-(3-(piperazin-1-yl)propoxy)-1H-indazol-3-yl)piperidine-2,6-dione (80 mg, 2.8 equiv., 0.21 mmol) and tert-butyl 6-(8-(benzo[d]thiazol-2-ylcarbamoyl)-3,4-dihydroisoquinolin-2(1H)-yl)-3-(3-(3-chloropropoxy)-2-methylphenyl)picolinate (50 mg, 1 equiv., 75 μmol) were dissolved in DMF (0.5 mL). Added DIEA (48 mg, 65 μL, 5 equiv., 0.37 mmol). Let mix at 50° C. overnight. Added an additional amount of DIEA (same amount as originally used). Let continue to mix overnight at 60° C. Purified reaction mixture by RP HPLC (water/MeCN, both with 0.1% formic acid modifier) to afford tert-butyl 6-(8-(benzo[d]thiazol-2-ylcarbamoyl)-3,4-dihydroisoquinolin-2(1H)-yl)-3-(3-(3-(4-(3-((3-(2,6-dioxopiperidin-3-yl)-1-methyl-1H-indazol-6-yl)oxy)propyl)piperazin-1-yl)propoxy)-2-methylphenyl)picolinate (12 mg, 12 μmol, 16% yield).

MS (ESI) m/z: 1018.2 [M+H]⁺.

### Step J. 6-(8-(Benzo[d]thiazol-2-ylcarbamoyl)-3,4-dihydroisoquinolin-2(1H)-yl)-3-(3-(3-(4-(3-((3-(2,6-dioxopiperidin-3-yl)-1-methyl-1H-indazol-6-yl)oxy)propyl)piperazin-1-yl)propoxy)-2-methylphenyl)picolinic acid

tert-Butyl 6-(8-(benzo[d]thiazol-2-ylcarbamoyl)-3,4-dihydroisoquinolin-2(1H)-yl)-3-(3-(3-(4-(3-((3-(2,6-dioxopiperidin-3-yl)-1-methyl-1H-indazol-6-yl)oxy)propyl)piperazin-1-yl)propoxy)-2-methylphenyl)picolinate (12 mg, 1 equiv., 12 μmol) was dissolved in a mixture of DCM (30 mL) and HCl in dioxane (0.21 g, 1.5 mL, 4.0 molar, 500 equiv., 5.9 mmol). Let reaction stir at ambient temperature for 72 h. The reaction mixture was concentrated under reduced pressure to afford 6-(8-(benzo[d]thiazol-2-ylcarbamoyl)-3,4-dihydroisoquinolin-2(1H)-yl)-3-(3-(3-(4-(3-((3-(2,6-dioxopiperidin-3-yl)-1-methyl-1H-indazol-6-yl)oxy)propyl)piperazin-1-yl)propoxy)-2-methylphenyl)picolinic acid (16.5 mg, 13.9 μmol).

MS (ESI) m/z: 962.2 [M+H]⁺.

### Example 6. Preparation of Compound 138a

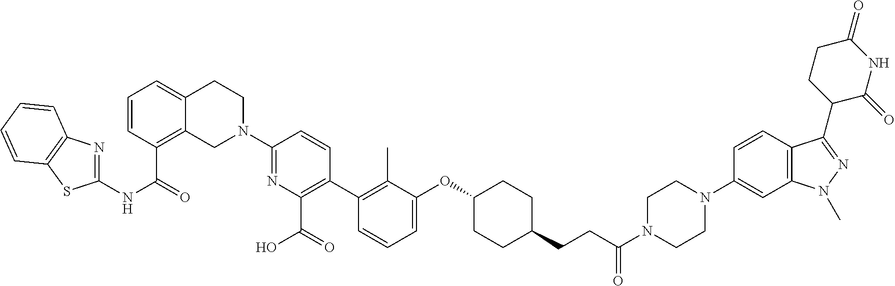 [SMILES: Cc1c(O[C@H]2CC[C@H](CCC(=O)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-6-yl)piperazin-1-yl)-3-oxopropyl)cyclohexyl)oxy)-2-methylphenyl)picolinic acid

### Step A. tert-Butyl 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-6-yl)piperazin-1-yl)-3-oxopropyl)cyclohexyl)oxy)-2-methylphenyl)picolinate

To a solution of 3-((1r,4r)-4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)cyclohexyl)propanoic acid (50 mg, 66.94 μmol, 1 equiv.), 3-(1-methyl-6-(piperazin-1-yl)-1H-indazol-3-yl)piperidine-2,6-dione (26.30 mg, 80.34 μmol, 1.2 equiv.) and EDCI (16.04 mg, 83.68 μmol, 1.2 equiv.) in pyridine (2 mL) was stirred at 25° C. for 3 hours. Then the mixture was diluted with water (2 mL) and extracted with ethyl acetate (1 mL×3). The combined organic layers were filtered and concentrated under reduced pressure to give tert-butyl 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-6-yl)piperazin-1-yl)-3-oxopropyl)cyclohexyl)oxy)-2-methylphenyl)picolinate (60 mg, crude) as a yellow oil which was carried forward without further purification.

MS (ESI) m/z: 1056.47 [M+H]⁺.

### Step B. 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-6-yl)piperazin-1-yl)-3-oxopropyl)cyclohexyl)oxy)-2-methylphenyl)picolinic acid

tert-Butyl 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-6-yl)piperazin-1-yl)-3-oxopropyl)cyclohexyl)oxy)-2-methylphenyl)picolinate (50 mg, 47.34 μmol, 1 equiv.) in trifluoroacetic acid (0.8 mL) and dichloromethane (0.8 mL) was stirred at 40° C. for 12 hours. The reaction mixture was concentrated under reduced pressure to give a residue. The residue was purified by prep-HPLC (column: Phenomenex luna C18 150×25 mm×10 μmol; mobile phase: [water (1% formic acid)-acetonitrile]; B %: 55%-85%, 10 min) to give 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-6-yl)piperazin-1-yl)-3-oxopropyl)cyclohexyl)oxy)-2-methylphenyl)picolinic acid (20.2 mg, 19.3 μmol, 40.7% yield, 95.2% purity) as a white solid.

MS (ESI) m/z: 1000.4 [M+H]⁺;

¹H NMR (400 MHz, DMSO-d6) δ 12.86-12.84 (m, 2H), 10.85 (s, 1H), 8.03 (d, J=8.0 Hz, 1H), 7.79 (d, J=8.0 Hz, 1H), 7.62 (d, J=7.2 Hz, 1H), 7.54-7.44 (m, 4H), 7.39-7.32 (m, 2H), 7.09-7.05 (m, 1H), 6.97-6.89 (m, 4H), 6.61 (d, J=7.2 Hz, 1H), 4.98 (s, 2H), 4.28-4.18 (m, 2H), 3.93-3.90 (m, 5H), 3.65-3.60 (m, 4H), 3.24-3.18 (m, 4H), 3.04-2.99 (m, 2H), 2.67-2.61 (m, 2H), 2.39 (t, J=7.6 Hz, 2H), 2.33-2.28 (m, 1H), 2.10-2.07 (m, 2H), 1.87-1.80 (m, 5H), 1.49-1.44 (m, 2H), 1.35-1.31 (m, 4H), 1.13-1.04 (m, 2H).

### Example 7. Preparation of Compound 142

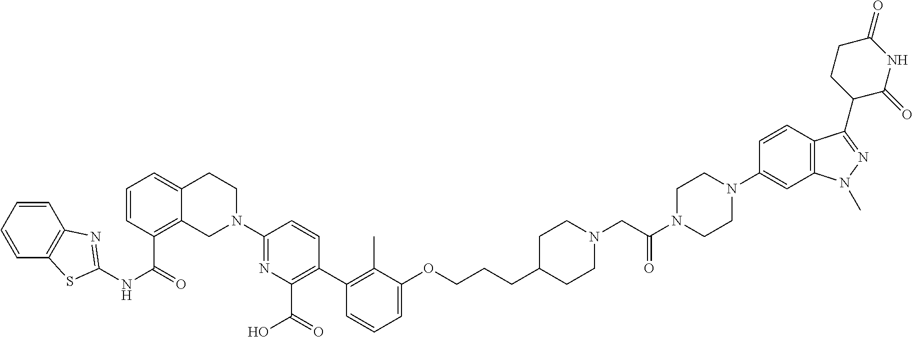 [SMILES: Cc1c(OCCCC2CCN(CC(=O)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-(1-(2-(4-(3-(2,6-dioxopiperidin-3-yl)-1-methyl-1H-indazol-6-yl)piperazin-1-yl)-2-oxoethyl)piperidin-4-yl)propoxy)-2-methylphenyl)picolinic acid

### Step A. tert-Butyl 6-(8-(benzo[d]thiazol-2-ylcarbamoyl)-3,4-dihydroisoquinolin-2(1H)-yl)-3-(3-(3-(1-(2-(4-(3-(2,6-dioxopiperidin-3-yl)-1-methyl-1H-indazol-6-yl)piperazin-1-yl)-2-oxoethyl)piperidin-4-yl)propoxy)-2-methylphenyl)picolinate

To a solution of 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-1-piperidyl]acetic acid (70 mg, 90.21 μmol, 1 equiv.) in DMF (2 mL) was added HATU (34.30 mg, 90.21 μmol, 1 equiv.), 3-(1-methyl-6-piperazin-1-yl-indazol-3-yl)piperidine-2,6-dione (29.53 mg, 90.21 μmol, 1 equiv.), and DIEA (11.66 mg, 90.21 μmol, 15.71 uL, 1 equiv.). The mixture was stirred at 25° C. for 2 hours. The reaction mixture was diluted with water (20 mL) and extracted with EtOAc (10×3 mL). The combined organic layers were filtered and concentrated under reduced pressure to give tert-butyl 6-(8-(benzo[d]thiazol-2-ylcarbamoyl)-3,4-dihydroisoquinolin-2(1H)-yl)-3-(3-(3-(1-(2-(4-(3-(2,6-dioxopiperidin-3-yl)-1-methyl-1H-indazol-6-yl)piperazin-1-yl)-2-oxoethyl)piperidin-4-yl)propoxy)-2-methylphenyl)picolinate (75 mg, 69.10 μmol, 76% yield) as a yellow oil, which was carried forward without further purification.

MS (ESI) m/z 543.4 [M12+H]⁺.

### Step B. 6-(8-(benzo[d]thiazol-2-ylcarbamoyl)-3,4-dihydroisoquinolin-2(1H)-yl)-3-(3-(3-(1-(2-(4-(3-(2,6-dioxopiperidin-3-yl)-1-methyl-1H-indazol-6-yl)piperazin-1-yl)-2-oxoethyl)piperidin-4-yl)propoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(3-(1-(2-(4-(3-(2,6-dioxopiperidin-3-yl)-1-methyl-1H-indazol-6-yl)piperazin-1-yl)-2-oxoethyl)piperidin-4-yl)propoxy)-2-methylphenyl)picolinate (70 mg, 64.50 μmol, 1 equiv.) in trifluoroacetic acid (1 mL) and dichloromethane (2 mL). The mixture was stirred at 25° C. for 12 hours. The reaction mixture was concentrated under reduced pressure to remove solvent to give the crude product. The crude product was purified by reverse-phase HPLC (column: Phenomenex luna C18 150×25 mm×10 um; mobile phase: [water (1% formic acid)-acetonitrile]; B %: 30%-60%, 10 min) to give 6-(8-(benzo[d]thiazol-2-ylcarbamoyl)-3,4-dihydroisoquinolin-2(1H)-yl)-3-(3-(3-(1-(2-(4-(3-(2,6-dioxopiperidin-3-yl)-1-methyl-1H-indazol-6-yl)piperazin-1-yl)-2-oxoethyl)piperidin-4-yl)propoxy)-2-methylphenyl)picolinic acid (25.64 mg, 23.93 μmol, 37% yield, 96% purity) as a white solid.

MS (ESI) m/z: (1029.7 [M+H]⁺;

¹H NMR (400 MHz, CDCl₃) δ 7.82-7.72 (m, 2H), 7.54 (d, J=7.6 Hz, 1H), 7.45 (dd, J=2.8, 8.8 Hz, 1H), 7.37-7.29 (m, 3H), 7.27-7.21 (m, 2H), 7.09-7.02 (m, 1H), 6.94-6.88 (m, 1H), 6.82-6.77 (m, 1H), 6.75-6.70 (m, 1H), 6.64-6.59 (m, 1H), 6.54 (s, 1H), 5.02 (s, 1H), 4.18 (t, J=6.0 Hz, 1H), 3.97-3.84 (m, 4H), 3.83 (d, J=2.0 Hz, 3H), 3.78-3.74 (m, 2H), 3.14 (d, J=7.6 Hz, 4H), 3.03 (d, J=4.0 Hz, 2H), 2.97-2.83 (m, 2H), 2.80 (d, J=100 Hz, 2H), 2.67-2.49 (m, 2H), 2.47-2.35 (m, 2H), 2.30 (dd, J=4.4, 8.8 Hz, 2H), 2.06-1.98 (m, 4H), 1.83 (s, 3H), 1.73-1.68 (m, 2H), 1.62 (d, J=12.4 Hz, 2H), 1.36-1.31 (m, 2H), 1.18 (s, 2H).

### Example 8. Preparation of Compound 148a

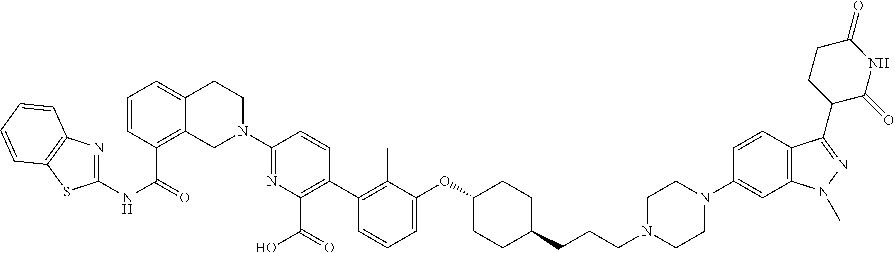 [SMILES: Cc1c(O[C@H]2CC[C@H](CCCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)piperazin-1-yl)propyl)cyclohexyl)oxy)-2-methylphenyl)picolinic acid

### Step A. tert-Butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)piperazin-1-yl)propyl)cyclohexyl)oxy)-2-methylphenyl)picolinate

A mixture of tert-butyl 6-[8-(1,3-benzothiazol-2-ylcarbamoyl)-3,4-dihydro-1H-isoquinolin-2-yl]-3-[2-methyl-3-[4-(3-oxopropyl)cyclohexoxy]phenyl]pyridine-2-carboxylate (80 mg, 109.45 μmol, 1 equiv), 3-(1-methyl-6-piperazin-1-yl-indazol-3-yl)piperidine-2,6-dione (43.00 mg, 131.34 μmol, 1.2 equiv.), 4A molecular sieves (109.45 μmol, 1 equiv.) and NaBH(OAc)₃ (69.59 mg, 328.35 μmol, 3 equiv.) in DCM (6 mL) was degassed and purged with N₂ three times, and then the mixture was stirred at 25° C. for 1.5 hour under nitrogen atmosphere. The reaction mixture was filtered and concentrated under reduced pressure to give tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)piperazin-1-yl)propyl)cyclohexyl)oxy)-2-methylphenyl)picolinate (100 mg, 95.94 μmol, 87% yield) as a white solid.

MS (ESI) m/z: 549.0 [M12+H]⁺.

### Step B. 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)piperazin-1-yl)propyl)cyclohexyl)oxy)-2-methylphenyl)picolinic acid

A solution of tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)piperazin-1-yl)propyl)cyclohexyl)oxy)-2-methylphenyl)picolinate (80 mg, 76.75 μmol, 1 equiv) in dichloromethane (1 mL) and trifluoroacetic acid (1 mL) was stirred at 40° C. After 3 h the reaction solution was concentrated and purified by prep-HPLC (column: Phenomenex luna C18 150×25 mm×10 um; mobile phase: [water (1% formic acid)-acetonitrile]; B %: 30%-60%, 0 min) to give 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)piperazin-1-yl)propyl)cyclohexyl)oxy)-2-methylphenyl)picolinic acid (70.56 mg, 66.37 μmol, 86% yield, 92.7% purity) as a white solid.

MS (ESI) m/z: 986.2 [M+H]⁺;

¹H NMR (400 MHz, DMSO-d6) δ 12.95-12.79 (m, 1H), 10.86 (s, 1H), 8.14 (s, 1H), 8.04 (d, J=7.6 Hz, 1H), 7.79 (d, J=8.0 Hz, 1H), 7.62 (d, J=7.6 Hz, 1H), 7.53-7.50 (m, 1H), 7.48-7.45 (m, 3H), 7.40-7.34 (m, 2H), 7.10-7.06 (m, 1H), 6.98-6.89 (m, 5H), 6.62 (d, J=7.2 Hz, 1H), 4.98 (s, 2H), 4.29-4.21 (m, 2H), 3.94-3.88 (m, 6H), 3.03 (t, J=5.2 Hz, 3H), 2.90-2.79 (m, 2H), 2.68-2.57 (m, 5H), 2.35-2.26 (m, 2H), 2.20-2.08 (m, 4H), 1.88 (s, 3H), 1.83-1.79 (m, 2H), 1.59-1.54 (m, 2H), 1.25 (d, J=6.8 Hz, 6H), 1.12-1.04 (m, 2H).

### Example 9. Preparation of Compound 154a

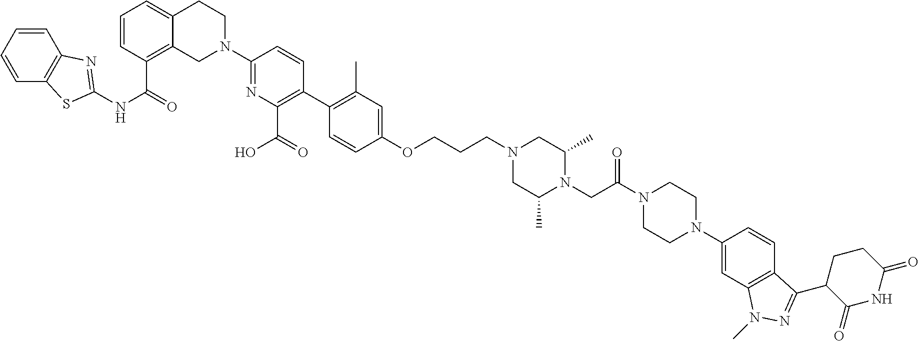 [SMILES: Cc1cc(OCCCN2C[C@@H](C)N(CC(=O)N3CCN(c4ccc5c(C6CCC(=O)NC6=O)nn(C)c5c4)CC3)[C@@H](C)C2)ccc1-c1ccc(N2CCc3cccc(C(=O)Nc4nc5ccccc5s4)c3C2)nc1C(=O)O]

### 6-(8-(Benzo[d]thiazol-2-ylcarbamoyl)-3,4-dihydroisoquinolin-2(1H)-yl)-3-(4-(3-((3R,5S)-4-(2-(4-(3-(2,6-dioxopiperidin-3-yl)-1-methyl-1H-indazol-6-yl)piperazin-1-yl)-2-oxoethyl)-3,5-dimethylpiperazin-1-yl)propoxy)-2-methylphenyl)picolinic acid

### Step A. (3R,5S)-tert-Butyl 4-(2-methoxy-2-oxoethyl)-3,5-dimethylpiperazine-1-carboxylate

A mixture of methyl 2-bromoacetate (9.35 g, 56.00 mmol, 6.19 mL, 1.5 equiv.), (3R, 5S)-tert-butyl 3,5-dimethylpiperazine-1-carboxylate (8 g, 37.33 mmol, 1 equiv.), and K₂CO₃ (15.48 g, 111.99 mmol, 3 equiv.) in MeCN (100 mL) was degassed and purged with N₂ three times, and then stirred at 60° C. After 3 hours, the reaction mixture was diluted with water (100 mL) and extracted with EtOAc (120 mL×3). The combined organic layers were washed with brine (120 mL×3), dried over anhydrous sodium sulfate, filtered, and concentrated under reduced pressure. The resulting crude material was purified by flash silica gel chromatography (0-25% ethyl acetate/petroleum ether) to give (3R,5S)-tert-butyl 4-(2-methoxy-2-oxoethyl)-3,5-dimethylpiperazine-1-carboxylate (10 g, 33.3 mmol, 89.2% yield) as a yellow oil.

¹H NMR (400 MHz, DMSO-d₆) δ=4.10-4.05 (m, 2H), 3.67 (s, 2H), 3.50 (s, 2H), 2.78-2.69 (m, 2H), 2.49-2.32 (m, 2H), 1.39 (s, 9H), 1.20-1.16 (m, 3H), 0.97 (d, J=6.4 Hz, 6H).

### Step B. 2-((2R,6S)-2,6-Dimethylpiperazin-1-yl)acetate hydrochloride

A mixture of (3R,5S)-tert-butyl 4-(2-methoxy-2-oxoethyl)-3,5-dimethylpiperazine-1-carboxylate (10 g, 33.29 mmol, 1 equiv.) and 4 M HCl in dioxane (83.22 mL, 10 equiv.) in dichloromethane (80 mL) was stirred at 25° C. for 16 hours under N₂ atmosphere. The reaction mixture was concentrated under reduced pressure to give methyl 2-((2R,6S)-2,6-dimethylpiperazin-1-yl)acetate hydrochloride (12.25 g, crude) as a yellow solid which was carried forward without further purification. ¹H NMR (400 MHz, CD₃OD) δ=4.44 (s, 2H), 4.36 (q, J=7.2 Hz, 2H), 4.29-4.21 (m, 2H), 3.74-3.67 (m, 2H), 3.58-3.50 (m, 2H), 1.49 (d, J=6.4 Hz, 6H), 1.35 (t, J=7.2 Hz, 3H).

### Step C. 1-Bromo-4-(3-bromopropoxy)-2-methylbenzene

A mixture of 4-bromo-3-methylphenol (3 g, 16.04 mmol, 1 equiv.), 1,3-dibromopropane (16.19 g, 80.20 mmol, 8.18 mL, 5 equiv.) and K₂CO₃ (6.65 g, 48.12 mmol, 3 equiv.) in MeCN (30 mL) was stirred at 70° C. for 12 hours under N₂ atmosphere. The reaction mixture was concentrated under reduced pressure and purified by flash silica gel chromatography (0-5% ethyl acetate/petroleum ether) to give 1-bromo-4-(3-bromopropoxy)-2-methylbenzene (2.5 g, 8.12 mmol, 50.6% yield) as a colorless oil. ¹H NMR (400 MHz, DMSO-d₆) δ=7.44 (d, J=8.8 Hz, 1H), 6.98 (d, J=2.8 Hz, 1H), 6.74 (dd, J=8.8, 2.8 Hz, 1H), 4.08-4.02 (m, 2H), 3.65 (t, J=6.4 Hz, 2H), 2.30 (s, 3H), 2.26-2.19 (m, 2H).

### Step D. Ethyl 2-((2R,6S)-4-(3-(4-bromo-3-methylphenoxy)propyl)-2,6-dimethylpiperazin-1-yl)acetate

A mixture of 1-bromo-4-(3-bromopropoxy)-2-methylbenzene (2.34 g, 7.60 mmol, 1.2 equiv.), methyl 2-((2R,6S)-2,6-dimethylpiperazin-1-yl)acetate hydrochloride (1.5 g, 6.34 mmol, 1 equiv.) and N,N-diisopropylethylamine (4.09 g, 31.68 mmol, 5.52 mL, 5 equiv.) in dimethylformamide (40 mL) was stirred at 60° C. for 16 hours under N₂ atmosphere. The reaction mixture was diluted with water (100 mL) and extracted with dichloromethane (100 mL×3). The combined organic layers were washed with brine (100 mL×6), dried over anhydrous sodium sulfate, filtered, and concentrated under reduced pressure. The resulting crude material was purified by flash silica gel chromatography (0˜7% dichloromethane/MeOH) to give ethyl 2-((2R,6S)-4-(3-(4-bromo-3-methylphenoxy)propyl)-2,6-dimethylpiperazin-1-yl)acetate (1.3 g, 3.04 mmol, 48% yield) as an orange oil. ¹H NMR (400 MHz, CD₃OD) δ=7.37 (d, J=8.8 Hz, 1H), 6.86 (d, J=3.0 Hz, 1H), 6.66 (dd, J=8.8, 3.2 Hz, 1H), 4.16 (q, J=7.2 Hz, 2H), 3.98 (t, J=6.0 Hz, 2H), 3.55 (s, 2H), 3.04-2.98 (m, 2H), 2.86-2.84 (m, 1H), 2.82 (s, 1H), 2.54-2.46 (m, 2H), 2.33 (s, 3H), 2.00-1.92 (m, 2H), 1.92-1.85 (m, 2H), 1.27 (t, J=7.2 Hz, 3H), 1.08 (d, J=6.4 Hz, 6H).

### Step E. tert-Butyl 6-(8-(benzo[d]thiazol-2-ylcarbamoyl)-3,4-dihydroisoquinolin-2(1H)-yl)-3-(4-(3-((3R,5S)-4-(2-ethoxy-2-oxoethyl)-3,5-dimethylpiperazin-1-yl)propoxy)-2-methylphenyl)picolinate

Ethyl 2-((2R,6S)-4-(3-(4-bromo-3-methylphenoxy)propyl)-2,6-dimethylpiperazin-1-yl)acetate (400 mg, 935.95 μmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (687.97 mg, 1.12 mmol, 1.2 equiv.), cataCXium® A Pd G3 (68.16 mg, 93.59 μmol, 0.1 equiv.) and KF (1.5 M, 1.87 mL, 3 equiv.) were taken up into a microwave tube in 1,4-dioxane (11 mL). The sealed tube was heated in a microwave reactor at 100° C. for 60 minutes. The reaction mixture was concentrated under reduced pressure and purified by reverse-phase HPLC to give tert-butyl 6-(8-(benzo[d]thiazol-2-ylcarbamoyl)-3,4-dihydroisoquinolin-2(1H)-yl)-3-(4-(3-((3R,5S)-4-(2-ethoxy-2-oxoethyl)-3,5-dimethylpiperazin-1-yl)propoxy)-2-methylphenyl)picolinate (410 mg, 477.46 umol, 51% yield, 97% purity) as a yellow solid. MS (ESI) m/z: 833.4 [M+H]⁺; ¹H NMR (400 MHz, DMSO-d₆) δ=13.12-12.55 (m, 1H), 8.02 (d, J=8.0 Hz, 1H), 7.77 (d, J=8.0 Hz, 1H), 7.60 (d, J=7.6 Hz, 1H), 7.48-7.41 (m, 3H), 7.39-7.31 (m, 2H), 6.92 (d, J=8.8 Hz, 1H), 6.85 (d, J=8.4 Hz, 1H), 6.81 (s, 1H), 6.75-6.69 (m, 1H), 4.96 (s, 2H), 4.06 (q, J=7.2 Hz, 2H), 3.98 (t, J=6.0 Hz, 2H), 3.86 (t, J=6.0 Hz, 2H), 3.45 (s, 2H), 3.03 (t, J=5.6 Hz, 2H), 2.87-2.80 (m, 2H), 2.72 (d, J=10.4 Hz, 2H), 2.34 (t, J=6.8 Hz, 2H), 2.00 (s, 3H), 1.87-1.78 (m, 2H), 1.66 (t, J=10.4 Hz, 2H), 1.17 (t, J=7.2 Hz, 3H), 1.03 (s, 9H), 0.95 (d, J=6.4 Hz, 6H).

### Step F. 2-((2R,6S)-4-(3-(4-(6-(8-(Benzo[d]thiazol-2-ylcarbamoyl)-3,4-dihydroisoquinolin-2(1H)-yl)-2-(tert-butoxycarbonyl)pyridin-3-yl)-3-methylphenoxy)propyl)-2,6-dimethylpiperazin-1-yl)acetic acid

A mixture of tert-butyl 6-(8-(benzo[d]thiazol-2-ylcarbamoyl)-3,4-dihydroisoquinolin-2(1H)-yl)-3-(4-(3-((3R,5S)-4-(2-ethoxy-2-oxoethyl)-3,5-dimethylpiperazin-1-yl)propoxy)-2-methylphenyl)picolinate (400 mg, 480.17 umol, 1 equiv.) and LiOH·H₂O (100.75 mg, 2.40 mmol, 5 equiv.) in THE (4 mL) and H₂O (1 mL) was stirred at 40° C. under N₂ atmosphere. After 7 hours, the reaction mixture was concentrated under reduced pressure to provide 2-((2R,6S)-4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)propyl)-2,6-dimethylpiperazin-1-yl)acetic acid (400 mg, crude) as a yellow solid, which was carried forward without further purification. MS (ESI) m/z: 805.7 [M+H]⁺.

### Step G. tert-Butyl 6-(8-(benzo[d]thiazol-2-ylcarbamoyl)-3,4-dihydroisoquinolin-2(1H)-yl)-3-(4-(3-((3R,5S)-4-(2-(4-(3-(2,6-dioxopiperidin-3-yl)-1-methyl-1H-indazol-6-yl)piperazin-1-yl)-2-oxoethyl)-3,5-dimethylpiperazin-1-yl)propoxy)-2-methylphenyl)picolinate

2-((2R,6S)-4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)propyl)-2,6-dimethylpiperazin-1-yl)acetic acid (100 mg, 124.22 μmol, 1 equiv.), 3-(1-methyl-6-(piperazin-1-yl)-1H-indazol-3-yl)piperidine-2,6-dione hydrochloride (54.24 mg, 149.07 μmol, 1.2 equiv.), and N,N-diisopropyl-N-diethylamine (48.17 mg, 372.67 μmol, 64.91 μL, 3 equiv.) in DMF (1.5 mL) was stirred at 25° C. After 5 minutes, HATU (56.68 mg, 149.07 umol, 1.2 equiv.) was added, and the mixture was stirred at 25° C. for 1 hour under N₂ atmosphere. The reaction mixture was concentrated under reduced pressure to give tert-butyl 6-(8-(benzo[d]thiazol-2-ylcarbamoyl)-3,4-dihydroisoquinolin-2(1H)-yl)-3-(4-(3-((3R,5S)-4-(2-(4-(3-(2,6-dioxopiperidin-3-yl)-1-methyl-1H-indazol-6-yl)piperazin-1-yl)-2-oxoethyl)-3,5-dimethylpiperazin-1-yl)propoxy)-2-methylphenyl)picolinate (130 mg, 116.7 μmol, 93.9% yield) as a brown solid. MS (ESI) m/z: 1114.5 [M+H]⁺.

### Step H. 6-(8-(Benzo[d]thiazol-2-ylcarbamoyl)-3,4-dihydroisoquinolin-2(1H)-yl)-3-(4-(3-((3R,5S)-4-(2-(4-(3-(2,6-dioxopiperidin-3-yl)-1-methyl-1H-indazol-6-yl)piperazin-1-yl)-2-oxoethyl)-3,5-dimethylpiperazin-1-yl)propoxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(4-(3-((3R,5S)-4-(2-(4-(3-(2,6-dioxopiperidin-3-yl)-1-methyl-1H-indazol-6-yl)piperazin-1-yl)-2-oxoethyl)-3,5-dimethylpiperazin-1-yl)propoxy)-2-methylphenyl)picolinate (120 mg, 107.69 μmol, 1 equiv.) in trifluoroacetic acid (0.6 mL) and dichloromethane (0.6 mL) was stirred at 25° C. After 16 h, the reaction mixture was concentrated under reduced pressure and purified by prep-HPLC (column: Phenomenex luna C18 150×25 mm×10 um; mobile phase: [water (1% formic acid)-acetonitrile]; B %: 25%-55%, 10 min) to give 6-(8-(benzo[d]thiazol-2-ylcarbamoyl)-3,4-dihydroisoquinolin-2(1H)-yl)-3-(4-(3-((3R,5S)-4-(2-(4-(3-(2,6-dioxopiperidin-3-yl)-1-methyl-1H-indazol-6-yl)piperazin-1-yl)-2-oxoethyl)-3,5-dimethylpiperazin-1-yl)propoxy)-2-methylphenyl)picolinic acid (80.4 mg, 73.8 μmol, 68.5% yield, 97.1% purity) as a white solid. MS (ESI) m/z: 1058.8 [M+H]⁺; ¹H NMR (400 MHz, DMSO-d₆) δ=13.38-12.38 (m, 1H), 10.85 (s, 1H), 8.14 (s, 1H), 8.03 (d, J=7.6 Hz, 1H), 7.79 (d, J=8.0 Hz, 1H), 7.62 (d, J=7.6 Hz, 1H), 7.52 (d, J=8.8 Hz, 1H), 7.50-7.31 (m, 6H), 6.96-6.88 (m, 4H), 6.78 (s, 1H), 6.73-6.68 (m, 1H), 4.97 (s, 2H), 4.29-4.24 (m, 1H), 3.98 (t, J=6.0 Hz, 2H), 3.90 (s, 3H), 3.69 (s, 3H), 3.61 (s, 6H), 3.23 (s, 2H), 3.19 (s, 2H), 3.13 (d, J=2.0 Hz, 2H), 3.02 (t, J=5.2 Hz, 3H), 2.79 (d, J=10.0 Hz, 2H), 2.65-2.59 (m, 2H), 2.45 (s, 2H), 2.34-2.26 (m, 1H), 2.20-2.13 (m, 1H), 2.03 (s, 3H), 1.90-1.85 (m, 2H), 0.97 (d, J=6.0 Hz, 6H).

### Example 10. Preparation of Compound 153

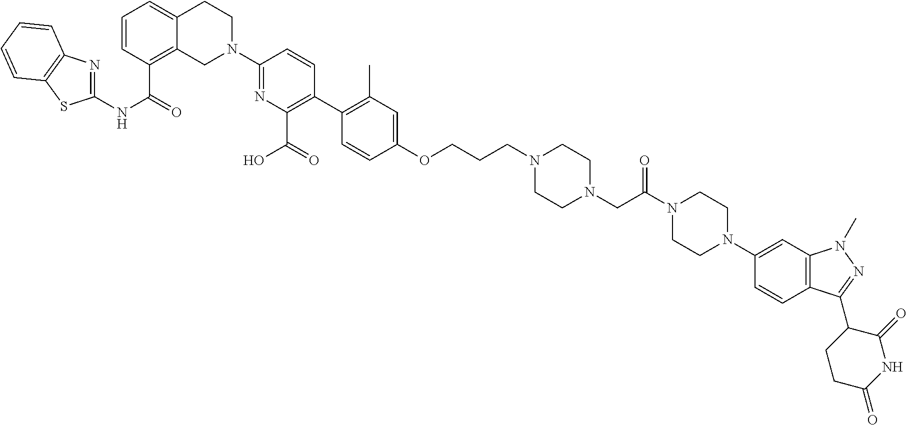 [SMILES: Cc1cc(OCCCN2CCN(CC(=O)N3CCN(c4ccc5c(C6CCC(=O)NC6=O)nn(C)c5c4)CC3)CC2)ccc1-c1ccc(N2CCc3cccc(C(=O)Nc4nc5ccccc5s4)c3C2)nc1C(=O)O]

### 6-(8-(Benzo[d]thiazol-2-ylcarbamoyl)-3,4-dihydroisoquinolin-2(1H)-yl)-3-(4-(3-(4-(2-(4-(3-(2,6-dioxopiperidin-3-yl)-1-methyl-1H-indazol-6-yl)piperazin-1-yl)-2-oxoethyl)piperazin-1-yl)propoxy)-2-methylphenyl)picolinic acid

### Step A. 1-Bromo-4-(3-bromopropoxy)-2-methylbenzene

A mixture of 4-bromo-3-methylphenol (2 g, 10.69 mmol, 1 equiv.), 1,3-dibromopropane (10.79 g, 53.47 mmol, 5.45 mL, 5 equiv.), and K₂CO₃ (4.43 g, 32.08 mmol, 3 equiv.) in MeCN (20 mL) was stirred at 70° C. After 12 h, the reaction mixture was filtered and concentrated under the reduced pressure. The resulting crude material was purified by flash silica gel chromatography (0-5% ethyl acetate/petroleum ether) to give 1-bromo-4-(3-bromopropoxy)-2-methylbenzene (1.55 g, 5.03 mmol, 47.1% yield) as a yellow oil.

¹H NMR (400 MHz, DMSO-d₆) δ=7.44 (d, J=8.8 Hz, 1H), 6.97 (d, J=2.8 Hz, 1H), 6.74 (dd, J=3.2, 8.8 Hz, 1H), 4.05 (t, J=6.0 Hz, 2H), 3.65 (t, J=6.4 Hz, 2H), 2.30 (s, 3H), 2.22 (m, J=6.4 Hz, 2H).

### Step B. Ethyl 2-(4-(3-(4-bromo-3-methylphenoxy)propyl)piperazin-1-yl)acetate

A mixture of 1-bromo-4-(3-bromopropoxy)-2-methylbenzene (1.5 g, 4.87 mmol, 1 equiv.), ethyl 2-(piperazin-1-yl)acetate (838.73 mg, 4.87 mmol, 1 equiv.), and K₂CO₃ (3.37 g, 24.35 mmol, 5 equiv.) in MeCN (15 mL) was stirred at 60° C. After 14 h, the reaction mixture was filtered and concentrated under the reduced pressure. The resulting crude material was purified by silica gel chromatography (0-100% ethyl acetate/petroleum ether) to give ethyl 2-(4-(3-(4-bromo-3-methylphenoxy)propyl)piperazin-1-yl)acetate (1.44 g, 3.28 mmol, 67.4% yield, 91.0% purity) as a yellow oil. ¹H NMR (400 MHz, DMSO-d₆) δ=7.53 (d, J=8.8 Hz, 1H), 7.05 (d, J=2.8 Hz, 1H), 6.82 (dd, J=3.2, 8.8 Hz, 1H), 4.19 (q, J=7.2 Hz, 2H), 4.07 (t, J=6.4 Hz, 2H), 3.29 (s, 2H), 2.62 (dd, J=2.0, 3.6 Hz, 4H), 2.56-2.45 (m, 6H), 2.41 (s, 3H), 1.99-1.91 (m, 2H), 1.30 (t, J=7.2 Hz, 3H).

### Step C. tert-Butyl 6-(8-(benzo[d]thiazol-2-ylcarbamoyl)-3,4-dihydroisoquinolin-2(1H)-yl)-3-(4-(3-(4-(2-ethoxy-2-oxoethyl)piperazin-1-yl)propoxy)-2-methylphenyl)picolinate

A mixture of ethyl 2-(4-(3-(4-bromo-3-methylphenoxy)propyl)piperazin-1-yl)acetate (400 mg, 1.00 mmol, 1 equiv.), tert-butyl 6-(8-((3H-indol-2-yl)carbamoyl)-3,4-dihydroisoquinolin-2(1H)-yl)-3-(4,4,5,5-tetramethyl-1,3,2-dioxaborolan-2-yl)picolinate (612.55 mg, 1.00 mmol, 1 equiv.), and K₂CO₃ (1.5 M, 1.00 mL, 1.5 equiv.) in dioxane (4 mL) was added cataCXium® A Pd G3 (145.65 mg, 200.00 μmol, 0.2 equiv.). The reaction mixture was purged with N₂ three times and the reaction mixture was stirred at 80° C. After 2 h, the reaction solution was partitioned between H₂O (10 mL) and ethyl acetate (10 mL). The organic phase was separated, washed with aqueous NaCl solution (3 mL×2), dried over with Na₂SO₄, filtered, and concentrated under reduced pressure. The resulting residue was purified by silica gel chromatography (0-90% ethyl acetate/petroleum ether) to give tert-butyl 6-(8-(benzo[d]thiazol-2-ylcarbamoyl)-3,4-dihydroisoquinolin-2(1H)-yl)-3-(4-(3-(4-(2-ethoxy-2-oxoethyl)piperazin-1-yl)propoxy)-2-methylphenyl)picolinate (692 mg, 733.27 μmol, 73.3% yield, 85.3% purity) as a yellow solid. ¹H NMR (400 MHz, DMSO-d₆) δ=13.01-12.64 (m, 1H), 8.02 (d, J=7.6 Hz, 1H), 7.77 (d, J=8.0 Hz, 1H), 7.60 (d, J=7.6 Hz, 1H), 7.46-7.41 (m, 3H), 7.39-7.31 (m, 2H), 6.94 (d, J=2.4 Hz, 1H), 6.86 (d, J=8.4 Hz, 1H), 6.81 (d, J=2.4 Hz, 1H), 6.73 (d, J=2.0 Hz, 1H), 4.96 (s, 2H), 4.04 (s, 2H), 4.02-4.00 (m, 2H), 3.86 (t, J=5.6 Hz, 2H), 3.17 (s, 2H), 3.03 (t, J=5.6 Hz, 2H), 2.39 (d, J=5.6 Hz, 8H), 2.33-2.25 (m, 3H), 2.00 (s, 2H), 1.83 (t, J=6.8 Hz, 2H), 1.18 (d, J=1.2 Hz, 3H), 1.03 (s, 9H).

### Step D. 2-(4-(3-(4-(6-(8-(Benzo[d]thiazol-2-ylcarbamoyl)-3,4-dihydroisoquinolin-2(1H)-yl)-2-(tert-butoxycarbonyl)pyridin-3-yl)-3-methylphenoxy)propyl)piperazin-1-yl)acetic acid

tert-Butyl 6-(8-(benzo[d]thiazol-2-ylcarbamoyl)-3,4-dihydroisoquinolin-2(1H)-yl)-3-(4-(3-(4-(2-ethoxy-2-oxoethyl)piperazin-1-yl)propoxy)-2-methylphenyl)picolinate (380 mg, 472.05 μmol, 1 equiv.) and LiOH·H₂O (1 M, 1.42 mL, 3 equiv.) in THE (4 mL) and H₂O (1 mL) was stirred at 25° C. After 13 h, the reaction solution was concentrated, diluted with water (4 mL), and the pH was adjusted to 3 by addition of 1 M HCl. The resulting slurry was filtered, and the filter cake was dried to give 2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)propyl)piperazin-1-yl)acetic acid (333 mg, crude) as a yellow solid which was carried forward without further purification. MS (ESI) m/z: 777.5 [M+H]⁺.

### Step E. tert-Butyl 6-(8-(benzo[d]thiazol-2-ylcarbamoyl)-3,4-dihydroisoquinolin-2(1H)-yl)-3-(4-(3-(4-(2-(4-(3-(2,6-dioxopiperidin-3-yl)-1-methyl-1H-indazol-6-yl)piperazin-1-yl)-2-oxoethyl)piperazin-1-yl)propoxy)-2-methylphenyl)picolinate

To a mixture of 2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)propyl)piperazin-1-yl)acetic acid (120 mg, 154.45 μmol, 1 equiv.), HATU (64.60 mg, 169.90 μmol, 1.1 equiv.), N,N-diisopropyl-N-ethylamine (59.9 mg, 463.4 μmol, 80.7 μL, 3 equiv.) in DMF (1.2 mL) was added 3-(1-methyl-6-(piperazin-1-yl)-1H-indazol-3-yl)piperidine-2,6-dione (50.6 mg, 154.5 μmol, 1 equiv.). The reaction solution was stirred at 25° C. After 2 h, the reaction solution was diluted with water (2 mL) and filtered. The filter cake was dissolved in dichloromethane and methanol and concentrated to provide tert-butyl 6-(8-(benzo[d]thiazol-2-ylcarbamoyl)-3,4-dihydroisoquinolin-2(1H)-yl)-3-(4-(3-(4-(2-(4-(3-(2,6-dioxopiperidin-3-yl)-1-methyl-1H-indazol-6-yl)piperazin-1-yl)-2-oxoethyl)piperazin-1-yl)propoxy)-2-methylphenyl)picolinate (215 mg, crude) as a yellow solid, which was carried forward without further purification. MS (ESI) m/z: 543.9 [M12+H]⁺.

### Step F. 6-(8-(Benzo[d]thiazol-2-ylcarbamoyl)-3,4-dihydroisoquinolin-2(1H)-yl)-3-(4-(3-(4-(2-(4-(3-(2,6-dioxopiperidin-3-yl)-1-methyl-1H-indazol-6-yl)piperazin-1-yl)-2-oxoethyl)piperazin-1-yl)propoxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(4-(3-(4-(2-(4-(3-(2,6-dioxopiperidin-3-yl)-1-methyl-1H-indazol-6-yl)piperazin-1-yl)-2-oxoethyl)piperazin-1-yl)propoxy)-2-methylphenyl)picolinate (215 mg, 197.92 μmol, 1 equiv.) in trifluoroacetic acid (1 mL) and CH₂Cl₂ (3 mL) was stirred at 25° C. After 70 h, the reaction mixture was concentrated under reduced pressure and purified by prep-HPLC (column: Phenomenex luna C18 150×25 mm×10 um; mobile phase: [water (1% formic acid)-MeCN]; B %: 20%-50%, 0 min) to give 6-(8-(benzo[d]thiazol-2-ylcarbamoyl)-3,4-dihydroisoquinolin-2(1H)-yl)-3-(4-(3-(4-(2-(4-(3-(2,6-dioxopiperidin-3-yl)-1-methyl-1H-indazol-6-yl)piperazin-1-yl)-2-oxoethyl)piperazin-1-yl)propoxy)-2-methylphenyl)picolinic acid (22.5 mg, 21.3 μmol, 10.8% yield, 97.4% purity) as a white solid. MS (ESI) m/z: 1030.7 [M+H]⁺; ¹H NMR (400 MHz, DMSO-d₆) δ=10.85 (s, 1H), 8.14 (s, 1H), 8.03 (d, J=8.0 Hz, 1H), 7.79 (d, J=8.0 Hz, 1H), 7.62 (d, J=7.6 Hz, 1H), 7.52 (d, J=8.8 Hz, 1H), 7.49-7.42 (m, 3H), 7.36 (q, J=8.0 Hz, 2H), 6.97-6.88 (m, 4H), 6.78 (d, J=2.0 Hz, 1H), 6.72-6.67 (m, 1H), 4.97 (s, 2H), 4.26 (dd, J=5.2, 9.3 Hz, 1H), 3.97 (t, J=6.4 Hz, 2H), 3.92 (s, 6H), 3.77-3.69 (m, 3H), 3.62 (s, 4H), 3.18 (d, J=4.0 Hz, 6H), 3.08-2.96 (m, 4H), 2.70-2.57 (m, 4H), 2.36-2.26 (m, 2H), 2.09 (d, J=18.0 Hz, 2H), 2.02 (s, 3H), 1.89-1.82 (m, 2H).

### Example 11. Preparation of Compound 152

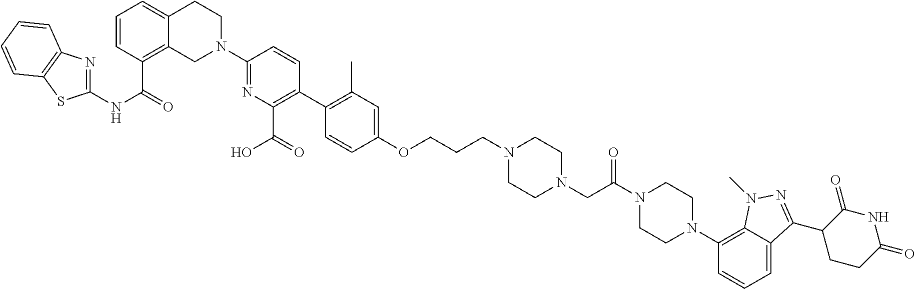 [SMILES: Cc1cc(OCCCN2CCN(CC(=O)N3CCN(c4cccc5c(C6CCC(=O)NC6=O)nn(C)c45)CC3)CC2)ccc1-c1ccc(N2CCc3cccc(C(=O)Nc4nc5ccccc5s4)c3C2)nc1C(=O)O]

### 6-(8-(Benzo[d]thiazol-2-ylcarbamoyl)-3,4-dihydroisoquinolin-2(1H)-yl)-3-(4-(3-(4-(2-(4-(3-(2,6-dioxopiperidin-3-yl)-1-methyl-1H-indazol-7-yl)piperazin-1-yl)-2-oxoethyl)piperazin-1-yl)propoxy)-2-methylphenyl)picolinic acid

### Step A. 1-Bromo-4-(3-bromopropoxy)-2-methylbenzene

A mixture of 4-bromo-3-methylphenol (2 g, 10.69 mmol, 1 equiv.), 1,3-dibromopropane (10.79 g, 53.47 mmol, 5.45 mL, 5 equiv.), and K₂CO₃ (4.43 g, 32.08 mmol, 3 equiv.) in MeCN (20 mL) was stirred at 70° C. After 12 h, the reaction mixture was filtered and concentrated under the reduced pressure. The resulting crude material was purified by flash silica gel chromatography (0-5% ethyl acetate/petroleum ether) to give 1-bromo-4-(3-bromopropoxy)-2-methylbenzene (1.55 g, 5.03 mmol, 47.1% yield) as a yellow oil.

¹H NMR (400 MHz, DMSO-d₆) δ=7.44 (d, J=8.8 Hz, 1H), 6.97 (d, J=2.8 Hz, 1H), 6.74 (dd, J=3.2, 8.8 Hz, 1H), 4.05 (t, J=6.0 Hz, 2H), 3.65 (t, J=6.4 Hz, 2H), 2.30 (s, 3H), 2.22 (m, J=6.4 Hz, 2H).

### Step B. Ethyl 2-(4-(3-(4-bromo-3-methylphenoxy)propyl)piperazin-1-yl)acetate

A mixture of 1-bromo-4-(3-bromopropoxy)-2-methylbenzene (1.5 g, 4.87 mmol, 1 equiv.), ethyl 2-(piperazin-1-yl)acetate (838.73 mg, 4.87 mmol, 1 equiv.), and K₂CO₃ (3.37 g, 24.35 mmol, 5 equiv.) in MeCN (15 mL) was stirred at 60° C. After 14 h, the reaction mixture was filtered and concentrated under the reduced pressure. The resulting crude material was purified by silica gel chromatography (0-100% ethyl acetate/petroleum ether) to give ethyl 2-(4-(3-(4-bromo-3-methylphenoxy)propyl)piperazin-1-yl)acetate (1.44 g, 3.28 mmol, 67.4% yield, 91.0% purity) as a yellow oil. ¹H NMR (400 MHz, DMSO-d₆) δ=7.53 (d, J=8.8 Hz, 1H), 7.05 (d, J=2.8 Hz, 1H), 6.82 (dd, J=3.2, 8.8 Hz, 1H), 4.19 (q, J=7.2 Hz, 2H), 4.07 (t, J=6.4 Hz, 2H), 3.29 (s, 2H), 2.62 (dd, J=2.0, 3.6 Hz, 4H), 2.56-2.45 (m, 6H), 2.41 (s, 3H), 1.99-1.91 (m, 2H), 1.30 (t, J=7.2 Hz, 3H).

### Step C. tert-Butyl 6-(8-(benzo[d]thiazol-2-ylcarbamoyl)-3,4-dihydroisoquinolin-2(1H)-yl)-3-(4-(3-(4-(2-ethoxy-2-oxoethyl)piperazin-1-yl)propoxy)-2-methylphenyl)picolinate

A mixture of ethyl 2-(4-(3-(4-bromo-3-methylphenoxy)propyl)piperazin-1-yl)acetate (400 mg, 1.00 mmol, 1 equiv.), tert-butyl 6-(8-((3H-indol-2-yl)carbamoyl)-3,4-dihydroisoquinolin-2(1H)-yl)-3-(4,4,5,5-tetramethyl-1,3,2-dioxaborolan-2-yl)picolinate (612.55 mg, 1.00 mmol, 1 equiv.), and K₂CO₃ (1.5 M, 1.00 mL, 1.5 equiv.) in dioxane (4 mL) was added cataCXium® A Pd G3 (145.65 mg, 200.00 μmol, 0.2 equiv.). The reaction mixture was purged with N₂ three times. The reaction mixture was stirred at 80° C. After 2 h, the reaction solution was partitioned between H₂O (10 mL) and ethyl acetate (10 mL). The organic phase was separated, washed with aqueous NaCl solution (3 mL×2), dried over with Na₂SO₄, filtered, and concentrated under reduced pressure. The resulting residue was purified by silica gel chromatography (0-90% ethyl acetate/petroleum ether) to give tert-butyl 6-(8-(benzo[d]thiazol-2-ylcarbamoyl)-3,4-dihydroisoquinolin-2(1H)-yl)-3-(4-(3-(4-(2-ethoxy-2-oxoethyl)piperazin-1-yl)propoxy)-2-methylphenyl)picolinate (692 mg, 733.27 μmol, 73.3% yield, 85.3% purity) as a yellow solid. ¹H NMR (400 MHz, DMSO-d₆) δ=13.01-12.64 (m, 1H), 8.02 (d, J=7.6 Hz, 1H), 7.77 (d, J=8.0 Hz, 1H), 7.60 (d, J=7.6 Hz, 1H), 7.46-7.41 (m, 3H), 7.39-7.31 (m, 2H), 6.94 (d, J=2.4 Hz, 1H), 6.86 (d, J=8.4 Hz, 1H), 6.81 (d, J=2.4 Hz, 1H), 6.73 (d, J=2.0 Hz, 1H), 4.96 (s, 2H), 4.04 (s, 2H), 4.02-4.00 (m, 2H), 3.86 (t, J=5.6 Hz, 2H), 3.17 (s, 2H), 3.03 (t, J=5.6 Hz, 2H), 2.39 (d, J=5.6 Hz, 8H), 2.33-2.25 (m, 3H), 2.00 (s, 2H), 1.83 (t, J=6.8 Hz, 2H), 1.18 (d, J=1.2 Hz, 3H), 1.03 (s, 9H).

### Step D. 2-(4-(3-(4-(6-(8-(Benzo[d]thiazol-2-ylcarbamoyl)-3,4-dihydroisoquinolin-2(1H)-yl)-2-(tert-butoxycarbonyl)pyridin-3-yl)-3-methylphenoxy)propyl)piperazin-1-yl)acetic acid

tert-Butyl 6-(8-(benzo[d]thiazol-2-ylcarbamoyl)-3,4-dihydroisoquinolin-2(1H)-yl)-3-(4-(3-(4-(2-ethoxy-2-oxoethyl)piperazin-1-yl)propoxy)-2-methylphenyl)picolinate (380 mg, 472.05 umol, 1 equiv.) and LiOH·H₂O (1 M, 1.42 mL, 3 equiv.) in THE (4 mL) and H₂O (1 ml) was stirred at 25° C. After 13 h, the reaction solution was concentrated, diluted with water (4 mL), and the pH was adjusted to 3 by the addition of 1 M HCl. The resulting slurry was filtered, and the filter cake was dried to give 2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)propyl)piperazin-1-yl)acetic acid (333 mg, crude) as a yellow solid which was carried forward without further purification. MS (ESI) m/z: 777.5 [M+H]⁺.

### Step E. tert-Butyl 6-(8-(benzo[d]thiazol-2-ylcarbamoyl)-3,4-dihydroisoquinolin-2(1H)-yl)-3-(4-(3-(4-(2-(4-(3-(2,6-dioxopiperidin-3-yl)-1-methyl-1H-indazol-7-yl)piperazin-1-yl)-2-oxoethyl)piperazin-1-yl)propoxy)-2-methylphenyl)picolinate

A mixture of 2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)propyl)piperazin-1-yl)acetic acid (170 mg, 218.81 μmol, 1 equiv.), HATU (91.52 mg, 240.69 umol, 1.1 equiv.), and N,N-diisopropyl-N-ethylamine (84.84 mg, 656.42 μmol, 114.34 μL, 3 equiv.) in DMF (2 mL) was added 3-(1-methyl-7-(piperazin-1-yl)-1H-indazol-3-yl)piperidine-2,6-dione (71.63 mg, 218.81 μmol, 1 equiv.), and the mixture was stirred at 25° C. After 12 h, the reaction solution was diluted with water (5 mL) and filtered. The filter cake was washed with dichloromethane (4 mL) and MeOH, and the filtrates were concentrated to provide tert-butyl 6-(8-(benzo[d]thiazol-2-ylcarbamoyl)-3,4-dihydroisoquinolin-2(1H)-yl)-3-(4-(3-(4-(2-(4-(3-(2,6-dioxopiperidin-3-yl)-1-methyl-1H-indazol-7-yl)piperazin-1-yl)-2-oxoethyl)piperazin-1-yl)propoxy)-2-methylphenyl)picolinate (213 mg, crude) as a yellow solid, which was carried forward without further purification. MS (ESI) m/z: 1086.8 [M+H]⁺.

### Step F. 6-(8-(Benzo[d]thiazol-2-ylcarbamoyl)-3,4-dihydroisoquinolin-2(1H)-yl)-3-(4-(3-(4-(2-(4-(3-(2,6-dioxopiperidin-3-yl)-1-methyl-1H-indazol-7-yl)piperazin-1-yl)-2-oxoethyl)piperazin-1-yl)propoxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(4-(3-(4-(2-(4-(3-(2,6-dioxopiperidin-3-yl)-1-methyl-1H-indazol-7-yl)piperazin-1-yl)-2-oxoethyl)piperazin-1-yl)propoxy)-2-methylphenyl)picolinate (200 mg, 184.11 μmol, 1 equiv.) in CH₂Cl₂ (3 mL) and trifluoracetic acid (1 mL) was stirred at 40° C. After 18 h, the reaction solution was concentrated and purified by prep-HPLC (column: Phenomenex luna C18 150×25 mm×10 um; mobile phase: [water (1% formic acid)-MeCN]; B %: 25%-55%, 10 min) to give 6-(8-(benzo[d]thiazol-2-ylcarbamoyl)-3,4-dihydroisoquinolin-2(1H)-yl)-3-(4-(3-(4-(2-(4-(3-(2,6-dioxopiperidin-3-yl)-1-methyl-1H-indazol-7-yl)piperazin-1-yl)-2-oxoethyl)piperazin-1-yl)propoxy)-2-methylphenyl)picolinic acid (64.5 mg, 55.16 μmol, 30.0% yield, 88.1% purity) as a green solid. MS (ESI) m/z: 1030.7 [M+H]⁺; ¹H NMR (400 MHz, DMSO-d₆) δ=10.96-10.83 (m, 1H), 8.13 (s, 1H), 8.03 (d, J=8.0 Hz, 1H), 7.79 (d, J=8.4 Hz, 1H), 7.62 (d, J=7.6 Hz, 1H), 7.50-7.41 (m, 5H), 7.40-7.34 (m, 2H), 7.06-7.02 (m, 2H), 6.94 (t, J=9.2 Hz, 2H), 6.79 (s, 1H), 6.72 (d, J=9.2 Hz, 1H), 4.97 (s, 2H), 4.57-4.37 (m, 2H), 4.35 (dd, J=5.2, 9.2 Hz, 1H), 4.27 (s, 3H), 4.01 (s, 4H), 3.91 (t, J=5.2 Hz, 2H), 3.02 (t, J=5.2 Hz, 4H), 2.98-2.92 (m, 2H), 2.87-2.75 (m, 4H), 2.73-2.65 (m, 4H), 2.64-2.56 (m, 4H), 2.38-2.27 (m, 2H), 2.24-2.09 (m, 2H), 2.03 (s, 5H).

### Example 12. Preparation of Compound 151

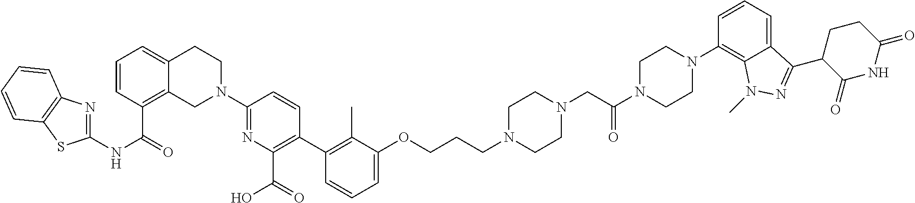 [SMILES: Cc1c(OCCCN2CCN(CC(=O)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-(8-(Benzo[d]thiazol-2-ylcarbamoyl)-3,4-dihydroisoquinolin-2(1H)-yl)-3-(3-(3-(4-(2-(4-(3-(2,6-dioxopiperidin-3-yl)-1-methyl-1H-indazol-7-yl)piperazin-1-yl)-2-oxoethyl)piperazin-1-yl)propoxy)-2-methylphenyl)picolinic acid

### Step A. tert-Butyl 6-(8-(benzo[d]thiazol-2-ylcarbamoyl)-3,4-dihydroisoquinolin-2(1H)-yl)-3-(3-(3-(4-(2-(4-(3-(2,6-dioxopiperidin-3-yl)-1-methyl-1H-indazol-7-yl)piperazin-1-yl)-2-oxoethyl)piperazin-1-yl)propoxy)-2-methylphenyl)picolinate

To a solution of 3-(1-methyl-7-(piperazin-1-yl)-1H-indazol-3-yl)piperidine-2,6-dione hydrochloride (50.56 mg, 139.0 μmol, 1.20 equiv.) in DMF (1 mL) was added N,N-diisopropyl-N-ethylamine (44.9 mg, 347.5 μmol, 60.53 μL, 3.00 equiv.), HOBt (20.4 mg, 150.6 μmol, 1.30 equiv.), EDCI (28.9 mg, 150.6 μmol, 1.30 equiv.), and 2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperazin-1-yl)acetic acid (90 mg, 115.8 μmol, 1.00 equiv.). The reaction solution was stirred at 25° C. After 12 h, the reaction mixture was concentrated under reduced pressure and purified by prep-TLC (SiO₂, DCM:MeOH=20:1) to give tert-butyl 6-(8-(benzo[d]thiazol-2-ylcarbamoyl)-3,4-dihydroisoquinolin-2(1H)-yl)-3-(3-(3-(4-(2-(4-(3-(2,6-dioxopiperidin-3-yl)-1-methyl-1H-indazol-7-yl)piperazin-1-yl)-2-oxoethyl)piperazin-1-yl)propoxy)-2-methylphenyl)picolinate (110 mg, 101.3 μmol, 87.4% yield) as a yellow oil. MS (ESI) m/z: 1086.8 [M+H]⁺.

### Step B. 6-(8-(Benzo[d]thiazol-2-ylcarbamoyl)-3,4-dihydroisoquinolin-2(1H)-yl)-3-(3-(3-(4-(2-(4-(3-(2,6-dioxopiperidin-3-yl)-1-methyl-1H-indazol-7-yl)piperazin-1-yl)-2-oxoethyl)piperazin-1-yl)propoxy)-2-methylphenyl)picolinic acid

A solution tert-butyl 6-(8-(benzo[d]thiazol-2-ylcarbamoyl)-3,4-dihydroisoquinolin-2(1H)-yl)-3-(3-(3-(4-(2-(4-(3-(2,6-dioxopiperidin-3-yl)-1-methyl-1H-indazol-7-yl)piperazin-1-yl)-2-oxoethyl)piperazin-1-yl)propoxy)-2-methylphenyl)picolinate (110 mg, 101.3 μmol, 1.00 equiv.) in dichloromethane (1 mL) and trifluoroacetic acid (1 mL) was stirred at 40° C. After 12 hours, the reaction mixture was concentrated and purified by prep-HPLC (column: Phenomenex luna C18 150*25 mm*10 um; mobile phase: [water (1% formic acid)-MeCN]; B %: 25%-55%, 10 min) to give 6-(8-(benzo[d]thiazol-2-ylcarbamoyl)-3,4-dihydroisoquinolin-2(1H)-yl)-3-(3-(3-(4-(2-(4-(3-(2,6-dioxopiperidin-3-yl)-1-methyl-1H-indazol-7-yl)piperazin-1-yl)-2-oxoethyl)piperazin-1-yl)propoxy)-2-methylphenyl)picolinic acid (22.8 mg, 21.1 μmol, 20.8% yield, 99.3% purity) as a white solid. MS (ESI) m/z: 1030.7 [M+H]⁺; ¹H NMR (400 MHz, DMSO) δ=10.89 (s, 1H), 8.03 (d, J=7.6 Hz, 1H), 7.79 (d, J=8.0 Hz, 1H), 7.62 (d, J=7.2 Hz, 1H), 7.49-7.33 (m, 7H), 7.09 (t, J=8.0 Hz, 1H), 7.02 (d, J=4.8 Hz, 2H), 6.97 (d, J=8.8 Hz, 1H), 6.88 (d, J=8.0 Hz, 1H), 6.63 (d, J=7.6 Hz, 1H), 4.98 (s, 2H), 4.47-4.40 (m, 1H), 4.34 (d, J=9.6 Hz, 1H), 4.27 (s, 3H), 4.22-4.15 (m, 1H), 3.99 (t, J=5.6 Hz, 2H), 3.91 (t, J=5.6 Hz, 2H), 3.18 (s, 8H), 3.02 (t, J=5.6 Hz, 3H), 2.95-2.89 (m, 2H), 2.80-2.74 (m, 2H), 2.62 (d, J=5.2 Hz, 2H), 2.54 (s, 4H), 2.36-2.29 (m, 2H), 2.16 (d, J=13.2 Hz, 1H), 1.92 (d, J=5.6 Hz, 2H), 1.89 (s, 3H).

### Example 13. Preparation of Compound 146a

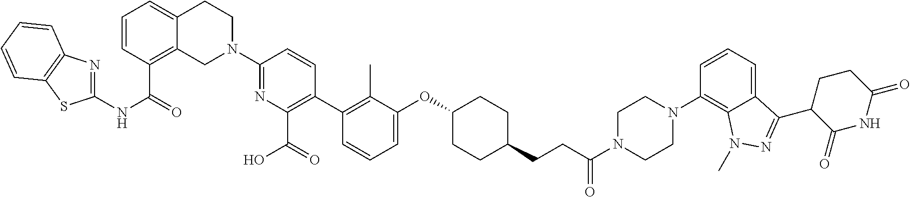 [SMILES: Cc1c(O[C@H]2CC[C@H](CCC(=O)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-7-yl)piperazin-1-yl)-3-oxopropyl)cyclohexyl)oxy)-2-methylphenyl)picolinic acid

### Step A. Ethyl (1r,4r)-4-(3-bromo-2-methylphenoxy)cyclohexane-1-carboxylate

To a solution of ethyl 4-hydroxycyclohexanecarboxylate (10 g, 58.06 mmol, 1 equiv.), 3-bromo-2-methyl-phenol (11.95 g, 63.87 mmol, 1.1 equiv.), and PPh₃ (24.37 g, 92.90 mmol, 1.6 equiv.) in THE (100 mL) was added DIAD (18.79 g, 92.90 mmol, 18.06 mL, 1.6 equiv.) under 0° C. Then the mixture was stirred at 25° C. for 16 h. The reaction mixture was concentrated under reduced pressure to give a residue that was purified by silica gel chromatography (ethyl acetate/petroleum ether gradient) to afford the title compound (3.6 g, 10.55 mmol, 18.17% yield). ¹H NMR (400 MHz, CDCl₃) δ 7.14 (d, J=8.0 Hz, 1H), 6.97 (t, J=8.0 Hz, 1H), 6.80 (d, J=8.0 Hz, 1H), 4.20-4.11 (m, 3H), 2.39-2.35 (m, 2H), 2.30 (s, 3H), 2.19-2.14 (m, 2H), 2.09-2.04 (m, 2H), 1.62-1.52 (m, 5H), 1.26 (t, J=6.8 Hz, 3H).

### Step B. ((1r,4r)-4-(3-Bromo-2-methylphenoxy)cyclohexyl)methanol

To a solution of ethyl 4-(3-bromo-2-methyl-phenoxy)cyclohexanecarboxylate (3.6 g, 10.55 mmol, 1 equiv.) in THE (15 mL) was added LiAlH₄ (480.49 mg, 12.66 mmol, 1.2 equiv.) under 0° C. The resulting mixture was then stirred at 25° C. for 1.5 h. The reaction was quenched by the addition of a saturated aqueous solution of sodium sulfate (10 mL), and then extracted with ethyl acetate (10 mL×3). The combined organic layers were dried over anhydrous sodium sulfate, filtered, and concentrated under reduced pressure to afford the title compound (2.96 g, crude) that was used without further purification. ¹H NMR (400 MHz, CDCl₃) δ 7.13 (d, J=8.0 Hz, 1H), 6.97 (t, J=8.0 Hz, 1H), 6.81 (d, J=8.4 Hz, 1H), 4.13-4.08 (m, 1H), 3.50 (d, J=6.4 Hz, 2H), 2.30 (s, 3H), 2.20-2.14 (m, 2H), 1.95-1.86 (m, 2H), 1.53-1.45 (m, 3H), 1.14-1.04 (m, 2H).

### Step C. (1r,4r)-4-(3-Bromo-2-methylphenoxy)cyclohexane-1-carbaldehyde

To a solution of DMSO (3.09 g, 39.57 mmol, 3.09 mL, 4 equiv.) in DCM (10 mL) was added dropwise a solution of oxalyl chloride (2.51 g, 19.79 mmol, 1.73 mL, 2 equiv.) in DCM (2 mL) at −70° C. under a nitrogen atmosphere. The mixture was stirred at −70° C. for 1 h. [4-(3-bromo-2-methyl-phenoxy)cyclohexyl]methanol (2.96 g, 9.89 mmol, 1 equiv.) in DCM (10 mL) was then added dropwise at −70° C. The solution was stirred for 1 h at −70° C. TEA (6.01 g, 59.36 mmol, 8.26 mL, 6 equiv.) was added, and the mixture was stirred at −70° C. for 0.5 h under a nitrogen atmosphere. The reaction mixture was quenched by the addition of water (10 mL), diluted with DCM (10 mL), and extracted with DCM (10 mL×3). The combined organic layers were washed with water (5 mL×3), dried over anhydrous sodium sulfate, filtered, and concentrated under reduced pressure to afford (1r,4r)-4-(3-bromo-2-methylphenoxy)cyclohexane-1-carbaldehyde (3 g, crude) that was used without further purification. ¹H NMR (400 MHz, DMSO-d6) δ 9.62-9.59 (m, 1H), 7.16-7.01 (m, 3H), 4.36-4.23 (m, 1H), 2.39-2.31 (m, 1H), 2.25-2.18 (m, 3H), 2.03-1.92 (m, 4H), 1.50-1.38 (m, 4H).

### Step D. Ethyl (E)-3-((1r,4r)-4-(3-bromo-2-methylphenoxy)cyclohexyl)acrylate

To a solution of NaH (56.52 mg, 1.41 mmol, 60% purity, 2.1 equiv.) in THE (3 mL) at 0° C. was added ethyl 2-diethoxyphosphorylacetate (301.75 mg, 1.35 mmol, 267.04 mL, 2 equiv.). (1r,4r)-4-(3-Bromo-2-methylphenoxy)cyclohexane-1-carbaldehyde (200 mg, 672.98 mmol, 1 equiv.) was then added, and the mixture was stirred at 25° C. for 6 h. The reaction mixture was quenched by addition of a saturated aqueous solution of ammonium chloride (5 mL) at 0° C., and then extracted with ethyl acetate (10 mL×3). The combined organic layers were washed with water (10 mL), dried over anhydrous sodium sulfate, filtered, and concentrated under reduced pressure to give a residue which was purified by silica gel chromatography (ethyl acetate/petroleum ether gradient) to afford ethyl (E)-3-((1r,4r)-4-(3-bromo-2-methylphenoxy)cyclohexyl)acrylate (140 mg, 362.12 mmol, 53.81% yield). ¹H NMR (400 MHz, CDCl₃) δ 7.14 (d, J=8.0 Hz, 1H), 7.01-6.87 (m, 2H), 6.80 (d, J=8.4 Hz, 1H), 5.82 (dd, J=1.2, 15.6 Hz, 1H), 4.21-4.15 (m, 2H), 4.16-4.07 (m, 1H), 2.30 (s, 3H), 2.26-2.16 (m, 3H), 1.97-1.89 (m, 2H), 1.58-1.48 (m, 2H), 1.33-1.26 (m, 5H).

### Step E. Ethyl 3-((1r,4r)-4-(3-bromo-2-methylphenoxy)cyclohexyl)propanoate

A mixture of ethyl (E)-3-[4-(3-bromo-2-methyl-phenoxy)cyclohexyl]prop-2-enoate (1.6 g, 4.36 mmol, 1 equiv.), PtO₂ (98.92 mg, 435.64 mmol, 0.1 equiv.) in EtOH (15 mL) was degassed and purged with hydrogen three times. The mixture was stirred at 25° C. for 3 hours under a hydrogen atmosphere (balloon, ˜15 psi). The reaction mixture was filtered and concentrated under reduced pressure to give a residue that was purified by preparative RP-HPLC (water/acetonitrile with 0.1% formic acid in each). Combined and lyophilized pure product containing fractions to afford ethyl 3-((1r,4r)-4-(3-bromo-2-methylphenoxy)cyclohexyl)propanoate (1.4 g, 3.79 mmol, 87.02% yield). ¹H NMR (400 MHz, DMSO-d6) δ 7.17-7.10 (m, 1H), 7.06 (t, J=8.0 Hz, 1H), 7.03-6.99 (m, 1H), 4.27-4.17 (m, 1H), 4.11-4.04 (m, 2H), 2.33-2.26 (m, 2H), 2.20 (s, 3H), 2.03 (d, J=10.0 Hz, 2H), 1.75 (d, J=12.0 Hz, 2H), 1.55-1.42 (m, 2H), 1.39-1.21 (m, 3H), 1.17 (t, J=7.2 Hz, 3H), 1.11-0.97 (m, 2H).

### Step F. tert-Butyl 6-(8-(benzo[d]thiazol-2-ylcarbamoyl)-3,4-dihydroisoquinolin-2(1H)-yl)-3-(3-(((1r,4r)-4-(3-ethoxy-3-oxopropyl)cyclohexyl)oxy)-2-methylphenyl)picolinate

A mixture of ethyl 3-[4-(3-bromo-2-methyl-phenoxy)cyclohexyl]propanoate (200 mg, 541.58 mmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (546.40 mg, 758.21 mmol, 1.4 equiv.), [2-(2-aminophenyl)phenyl]palladium(1+); bis(1-adamantyl)-butyl-phosphane; methanesulfonate (78.88 mg, 108.32 mmol, 0.2 equiv.), and an aqueous solution of potassium carbonate (1.5 M, 541.58 mL, 1.5 equiv.) in dioxane (2.5 mL) was degassed and purged with nitrogen three times. The mixture was stirred at 100° C. for 1 h under microwave heating. The reaction mixture was filtered and concentrated under reduced pressure to provide a residue that was purified by silica gel chromatography (ethyl acetate/petroleum ether gradient) to afford the title compound (440 mg, crude). ¹H NMR (400 MHz, DMSO-d6) δ 8.02 (d, J=7.6 Hz, 1H), 7.78 (d, J=8.0 Hz, 1H), 7.60 (d, J=7.5 Hz, 1H), 7.50-7.40 (m, 3H), 7.40-7.30 (m, 2H), 7.12-7.03 (m, 1H), 6.99-6.89 (m, 2H), 6.59-6.49 (m, 1H), 5.03-4.90 (m, 2H), 4.27-4.14 (m, 1H), 4.03 (d, J=6.8 Hz, 2H), 3.87 (t, J=5.6 Hz, 2H), 3.29 (s, 2H), 3.03 (t, J=5.6 Hz, 2H), 2.29 (t, J=7.6 Hz, 2H), 2.05 (d, J=11.2 Hz, 1H), 1.84 (s, 3H), 1.79-1.70 (m, 2H), 1.50-1.42 (m, 2H), 1.23 (d, J=8.4 Hz, 2H), 1.19-1.17 (m, 3H), 1.07 (s, 2H), 1.00 (s, 9H).

### Step G. 3-((1r,4r)-4-(3-(6-(8-(Benzo[d]thiazol-2-ylcarbamoyl)-3,4-dihydroisoquinolin-2(1H)-yl)-2-(tert-butoxycarbonyl)pyridin-3-yl)-2-methylphenoxy)cyclohexyl)propanoic acid

A mixture of tert-butyl 6-(8-(benzo[d]thiazol-2-ylcarbamoyl)-3,4-dihydroisoquinolin-2(1H)-yl)-3-(3-(((1r,4r)-4-(3-ethoxy-3-oxopropyl)cyclohexyl)oxy)-2-methylphenyl)picolinate (440 mg, 567.77 mmol, 1 equiv.) and LiOH monohydrate (71.47 mg, 1.70 mmol, 3 equiv.) in THE (1.2 mL) and water (0.4 mL) was stirred at 25° C. for 1.5 h. The mixture was concentrated and redissolved in water (20 mL), and an aqueous solution of 1M HCl was added to adjust the pH to 2. The resulting solids were filtered and dried under reduced pressure to afford the title compound (380 mg, 508.76 mmol, 89.61% yield).

### Step H. tert-butyl 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-7-yl)piperazin-1-yl)-3-oxopropyl)cyclohexyl)oxy)-2-methylphenyl)picolinate

A mixture of 3-((1r,4r)-4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)cyclohexyl)propanoic acid (100 mg, 133.88 umol, 1 equiv.), 3-(1-methyl-7-piperazin-1-yl-indazol-3-yl)piperidine-2,6-dione (48.21 mg, 147.27 umol, 1.1 equiv.), and EDCI (32.08 mg, 167.35 μmol, 1.25 equiv.) in pyridine (1 mL) was stirred at 25° C. for 12 hours. The mixture was diluted with water (2 mL) and extracted with ethyl acetate (1 mL×3). The combined organic layers were filtered and concentrated under reduced pressure to give tert-butyl 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-7-yl)piperazin-1-yl)-3-oxopropyl)cyclohexyl)oxy)-2-methylphenyl)picolinate (120 mg, crude) as a yellow oil which was carried forward without further purification.

MS(ESI) m/z: 1056.7 [M+H]⁺.

### Step I. 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-7-yl)piperazin-1-yl)-3-oxopropyl)cyclohexyl)oxy)-2-methylphenyl)picolinic acid

tert-butyl 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-7-yl)piperazin-1-yl)-3-oxopropyl)cyclohexyl)oxy)-2-methylphenyl)picolinate (100 mg, 94.67 umol, 1 eq.) in trifluoroacetic acid (0.5 mL) and dichloromethane (0.5 mL) was stirred at 40° C. for 2 hours. The reaction mixture was concentrated under reduced pressure to give a residue. The resulting residue was purified by prep-HPLC (column: Phenomenex luna C18 150×25 mm×10 um; mobile phase: [water (1% formic acid)-MeCN]; 63%-93%, 15 min) to give 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-7-yl)piperazin-1-yl)-3-oxopropyl)cyclohexyl)oxy)-2-methylphenyl)picolinic acid (31.1 mg, 28.2 umol, 29.8% yield) as a yellow solid. MS(ESI) m/z: 1000.6 [M+H]⁺; ¹HNMR (400 MHz, DMSO-d₆) δ=12.95-12.80 (m, 1H), 12.75-12.42 (m, 1H), 10.94-10.85 (m, 1H), 8.03 (d, J=7.6 Hz, 1H), 7.79 (d, J=8.0 Hz, 1H), 7.66-7.59 (m, 1H), 7.48-7.34 (m, 6H), 7.11-7.01 (m, 3H), 6.94 (dd, J=8.4, 15.6 Hz, 2H), 6.61 (d, J=7.6 Hz, 1H), 4.97 (s, 2H), 4.37-4.31 (m, 1H), 4.27 (s, 3H), 4.23-4.14 (m, 1H), 4.07-3.81 (m, 3H), 3.25-3.14 (m, 2H), 3.10-3.00 (m, 2H), 2.96-2.84 (m, 1H), 2.84-2.73 (m, 1H), 2.70-2.60 (m, 3H), 2.41-2.31 (m, 3H), 2.21-2.02 (m, 4H), 1.89-1.79 (m, 5H), 1.50-1.43 (m, 2H), 1.42-1.25 (m, 4H), 1.14-1.03 (m, 2H).

### Example 14. Preparation of Compound 143

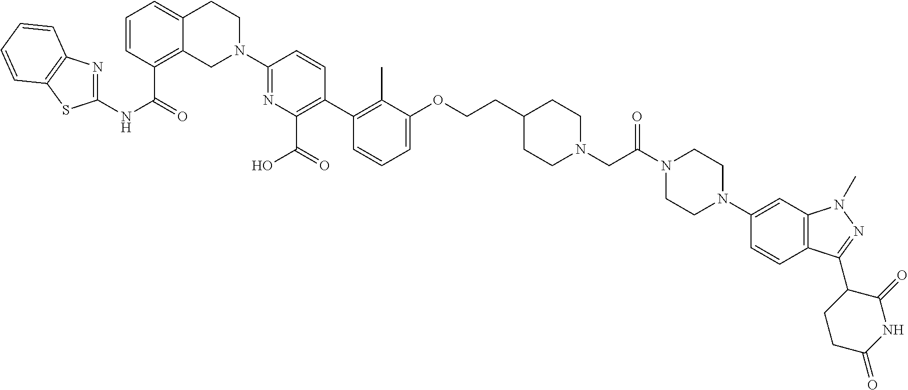 [SMILES: Cc1c(OCCC2CCN(CC(=O)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(2-(1-(2-(4-(3-(2,6-dioxopiperidin-3-yl)-1-methyl-1H-indazol-6-yl)piperazin-1-yl)-2-oxoethyl)piperidin-4-yl)ethoxy)-2-methylphenyl)picolinic acid

### Step A. tert-Butyl 6-(8-(benzo[d]thiazol-2-ylcarbamoyl)-3,4-dihydroisoquinolin-2(1H)-yl)-3-(3-(2-(1-(2-ethoxy-2-oxoethyl)piperidin-4-yl)ethoxy)-2-methylphenyl)picolinate

A mixture of tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (300 mg, 489.8 μmol, 1.00 equiv.), ethyl 2-(4-(2-(3-bromo-2-methylphenoxy)ethyl)piperidin-1-yl)acetate (207.0 mg, 538.7 μmol, 1.10 equiv.), cataCXium Pd G3 (35.7 mg, 49.0 μmol, 0.10 equiv.), and KF (85.37 mg, 1.47 mmol, 34.4 μL, 3.00 equiv.) in dioxane (4 mL) and water (1 mL) was stirred at 100° C. under N₂ atmosphere. After 1 h, the reaction solution was concentrated and purified by prep-TLC (SiO₂, petroleum ether: ethyl acetate=1:1, Rf=0.12) to give tert-butyl 6-(8-(benzo[d]thiazol-2-ylcarbamoyl)-3,4-dihydroisoquinolin-2(1H)-yl)-3-(3-(2-(1-(2-ethoxy-2-oxoethyl)piperidin-4-yl)ethoxy)-2-methylphenyl)picolinate (360 mg, 455.7 μmol, 93.1% yield) as a yellow solid. MS (ESI) m/z: 790.7 [M+H]⁺.

### Step B. 2-(4-(2-(3-(6-(8-(Benzo[d]thiazol-2-ylcarbamoyl)-3,4-dihydroisoquinolin-2(1H)-yl)-2-(tert-butoxycarbonyl)pyridin-3-yl)-2-methylphenoxy)ethyl)piperidin-1-yl)acetic acid

To a solution of tert-butyl 6-(8-(benzo[d]thiazol-2-ylcarbamoyl)-3,4-dihydroisoquinolin-2(1H)-yl)-3-(3-(2-(1-(2-ethoxy-2-oxoethyl)piperidin-4-yl)ethoxy)-2-methylphenyl)picolinate (360 mg, 455.7 μmol, 1.00 equiv.) in THE (4 mL) and H₂O (1 mL) was added LiOH·H₂O (57.4 mg, 1.37 mmol, 3.00 equiv.). The mixture was stirred at 25° C. After 12 h, the reaction solution was acidified to pH=5 with 1M HCl and extracted with EtOAc (20 mL×3). The combined organic layers were washed with brine (30 mL×1), dried over Na₂SO₄, filtered, and concentrated to provide 2-(4-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethyl)piperidin-1-yl)acetic acid (300 mg, 393.74 umol, 86.4% yield) as a yellow solid, which was carried forward without further purification. MS (ESI) m/z: 762.6 [M+H]⁺.

### Step C. tert-Butyl 6-(8-(benzo[d]thiazol-2-ylcarbamoyl)-3,4-dihydroisoquinolin-2(1H)-yl)-3-(3-(2-(1-(2-(4-(3-(2,6-dioxopiperidin-3-yl)-1-methyl-1H-indazol-6-yl)piperazin-1-yl)-2-oxoethyl)piperidin-4-yl)ethoxy)-2-methylphenyl)picolinate

To a solution of 2-(4-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethyl)piperidin-1-yl)acetic acid (100 mg, 131.3 μmol, 1.00 equiv.) in DMF (1 mL) was added N,N-diisopropyl-N-ethylamine (50.9 mg, 393.7 μmol, 68.6 μL, 3.00 equiv.), HATU (59.9 mg, 157.5 μmol, 1.20 equiv.), and 3-(1-methyl-6-piperazin-1-yl-indazol-3-yl)piperidine-2,6-dione hydrochloride (52.5 mg, 144.4 μmol, 1.10 equiv.). The reaction solution was stirred at 25° C. After 1 h, the reaction solution was diluted with water (20 mL) and extracted with dichloromethane (30 mL×3). The combined organic layers were washed with brine, dried over Na₂SO₄, filtered, and concentrated to give tert-butyl 6-(8-(benzo[d]thiazol-2-ylcarbamoyl)-3,4-dihydroisoquinolin-2(1H)-yl)-3-(3-(2-(1-(2-(4-(3-(2,6-dioxopiperidin-3-yl)-1-methyl-1H-indazol-6-yl)piperazin-1-yl)-2-oxoethyl)piperidin-4-yl)ethoxy)-2-methylphenyl)picolinate (120 mg, 112.01 umol, 85.35% yield) as a yellow solid, which was carried forward without further purification. MS (ESI) m/z: 1071.8 [M+H]⁺.

### Step D. 6-(8-(Benzo[d]thiazol-2-ylcarbamoyl)-3,4-dihydroisoquinolin-2(1H)-yl)-3-(3-(2-(1-(2-(4-(3-(2,6-dioxopiperidin-3-yl)-1-methyl-1H-indazol-6-yl)piperazin-1-yl)-2-oxoethyl)piperidin-4-yl)ethoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(2-(1-(2-(4-(3-(2,6-dioxopiperidin-3-yl)-1-methyl-1H-indazol-6-yl)piperazin-1-yl)-2-oxoethyl)piperidin-4-yl)ethoxy)-2-methylphenyl)picolinate (120 mg, 112.01 μmol, 1.00 equiv.) in DCM (1 mL) was added trifluoroacetic acid (1.54 g, 13.51 mmol, 1 mL, 120.6 equiv.). The reaction mixture was stirred at 40° C. After 1 h the reaction solution was concentrated and purified by prep-HPLC (column: Phenomenex luna C¹⁸ 150×25 mm×10 um; mobile phase: [water (1% formic acid)-acetonitrile]; B %: 25%-55%, 10 min) to give 6-(8-(benzo[d]thiazol-2-ylcarbamoyl)-3,4-dihydroisoquinolin-2(1H)-yl)-3-(3-(2-(1-(2-(4-(3-(2,6-dioxopiperidin-3-yl)-1-methyl-1H-indazol-6-yl)piperazin-1-yl)-2-oxoethyl)piperidin-4-yl)ethoxy)-2-methylphenyl)picolinic acid (23.1 mg, 22.2 μmol, 19.8% yield, 97.8% purity) as a white solid. MS (ESI) m/z: 1015.8 [M+H]⁺; ¹H NMR (400 MHz, DMSO-d₆) δ=10.86 (s, 1H), 8.04 (d, J=8.0 Hz, 1H), 7.79 (d, J=7.6 Hz, 1H), 7.63 (d, J=7.2 Hz, 1H), 7.53 (d, J=8.8 Hz, 1H), 7.50-7.32 (m, 6H), 7.12-7.05 (m, 1H), 6.95 (d, J=8.8 Hz, 2H), 6.91-6.86 (m, 2H), 6.63 (d, J=7.6 Hz, 1H), 4.98 (s, 2H), 4.29-4.25 (m, 1H), 4.02-3.98 (m, 2H), 3.92 (s, 2H), 3.90 (s, 3H), 3.75 (s, 2H), 3.63 (s, 2H), 3.26-3.25 (m, 2H), 3.18 (s, 4H), 3.03 (t, J=5.6 Hz, 2H), 2.84 (d, J=11.6 Hz, 2H), 2.63-2.59 (m, 2H), 2.33-2.25 (m, 1H), 2.21-2.13 (m, 1H), 2.05-1.97 (m, 2H), 1.89 (s, 3H), 1.74-1.74 (m, 1H), 1.74-1.66 (m, 4H), 1.54-1.47 (m, 1H), 1.28-1.20 (m, 2H)

### Example 15. Preparation of Compound 140

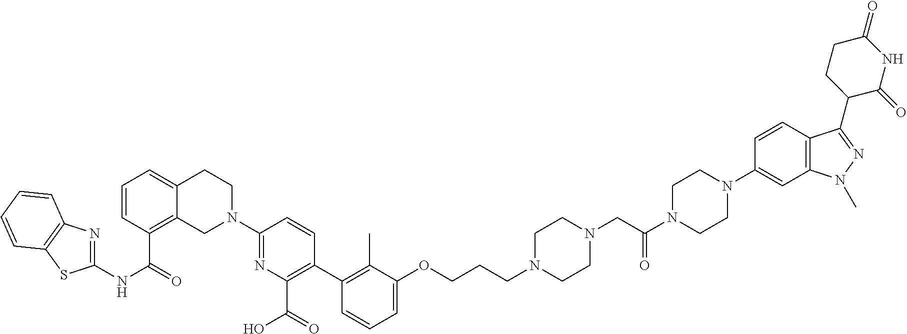 [SMILES: Cc1c(OCCCN2CCN(CC(=O)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(Benzo[d]thiazol-2-ylcarbamoyl)-3,4-dihydroisoquinolin-2(1H)-yl)-3-(3-(3-(4-(2-(4-(3-(2,6-dioxopiperidin-3-yl)-1-methyl-1H-indazol-6-yl)piperazin-1-yl)-2-oxoethyl)piperazin-1-yl)propoxy)-2-methylphenyl)picolinic acid

### Step A. tert-Butyl 6-(8-(benzo[d]thiazol-2-ylcarbamoyl)-3,4-dihydroisoquinolin-2(1H)-yl)-3-(3-(3-(4-(2-ethoxy-2-oxoethyl)piperazin-1-yl)propoxy)-2-methylphenyl)picolinate

To a solution of ethyl 2-(4-(3-(3-bromo-2-methylphenoxy)propyl)piperazin-1-yl)acetate (201.5 mg, 504.6 μmol, 1.00 equiv.) in 1,4-dioxane (6 mL) and H₂O (0.6 mL) were added cataCXium Pd G3 (36.75 mg, 50.5 μmol, 0.10 equiv.), KF (87.95 mg, 1.51 mmol, 35.46 μL, 3.00 equiv.) and tert-butyl 6-(8-((3H-indol-2-yl)carbamoyl)-3,4-dihydroisoquinolin-2(1H)-yl)-3-(4,4,5,5-tetramethyl-1,3,2-dioxaborolan-2-yl)picolinate (300 mg, 504.62 μmol, 1 equiv.). The mixture was stirred at 100° C. for 1 hour under nitrogen atmosphere. The reaction mixture was concentrated and purified by prep-TLC (SiO₂, DCM:MeOH=20:1, Rf=0.4) to give tert-butyl 6-(8-(benzo[d]thiazol-2-ylcarbamoyl)-3,4-dihydroisoquinolin-2(1H)-yl)-3-(3-(3-(4-(2-ethoxy-2-oxoethyl)piperazin-1-yl)propoxy)-2-methylphenyl)picolinate (220 mg, 273.29 μmol, 54.2% yield) as a yellow oil. MS (ESI) m/z: 805.4 [M+H]⁺.

### Step B. 2-(4-(3-(3-(6-(8-(Benzo[d]thiazol-2-ylcarbamoyl)-3,4-dihydroisoquinolin-2(1H)-yl)-2-(tert-butoxycarbonyl)pyridin-3-yl)-2-methylphenoxy) propyl)piperazin-1-yl)acetic acid

To a solution of tert-butyl 6-(8-(benzo[d]thiazol-2-ylcarbamoyl)-3,4-dihydroisoquinolin-2(1H)-yl)-3-(3-(3-(4-(2-ethoxy-2-oxoethyl)piperazin-1-yl)propoxy)-2-methylphenyl)picolinate (220 mg, 273.29 μmol, 1.00 equiv.) in THF (4 mL) was added LiOH·H₂O (19.6 mg, 819.88 μmol, 3.00 equiv.) and H₂O (1 mL). The reaction mixture was stirred at 25° C. After 12 h, the reaction solution was acidified to pH=3 with aqueous citric acid, filtered, and concentrated to provide 2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy) propyl)piperazin-1-yl)acetic acid (175 mg, 225.2 μmol, 82.4% yield) as a yellow solid, which was carried forward without further purification. MS (ESI) m/z: 777.6 [M+H]⁺.

### Step C. tert-Butyl 6-(8-(benzo[d]thiazol-2-ylcarbamoyl)-3,4-dihydroisoquinolin-2(1H)-yl)-3-(3-(3-(4-(2-(4-(3-(2,6-dioxopiperidin-3-yl)-1-methyl-1H-indazol-6-yl)piperazin-1-yl)-2-oxoethyl)piperazin-1-yl)propoxy)-2-methylphenyl)picolinate

To a solution of 3-(1-methyl-6-(piperazin-1-yl)-1H-indazol-3-yl)piperidine-2,6-dione hydrochloride (41.21 mg, 113.27 μmol, 1.10 equiv.) and 2-(4-(3-(3-(6-(8-(Benzo[d]thiazol-2-ylcarbamoyl)-3,4-dihydroisoquinolin-2(1H)-yl)-2-(tert-butoxycarbonyl)pyridin-3-yl)-2-methylphenoxy) propyl)piperazin-1-yl)acetic acid (80 mg, 102.97 umol, 1.00 equiv.) in DMF (1 mL) were added N,N-diisopropyl-N-ethylamine (39.9 mg, 308.9 μmol, 53.80 μL, 3.00 equiv.) and HATU (58.7 mg, 154.5 μmol, 1.50 equiv.). The reaction solution was stirred at 25° C. After 1 h, the reaction solution was concentrated under reduced pressure to give tert-butyl 6-(8-(benzo[d]thiazol-2-ylcarbamoyl)-3,4-dihydroisoquinolin-2(1H)-yl)-3-(3-(3-(4-(2-(4-(3-(2,6-dioxopiperidin-3-yl)-1-methyl-1H-indazol-6-yl)piperazin-1-yl)-2-oxoethyl)piperazin-1-yl)propoxy)-2-methylphenyl)picolinate (110 mg, 101.26 μmol, 98.3% yield) as a yellow solid, which was carried forward without further purification. MS (ESI) m/z: 1086.7 [M+H]⁺.

### Step D. 6-(8-(Benzo[d]thiazol-2-ylcarbamoyl)-3,4-dihydroisoquinolin-2(1H)-yl)-3-(3-(3-(4-(2-(4-(3-(2,6-dioxopiperidin-3-yl)-1-methyl-1H-indazol-6-yl)piperazin-1-yl)-2-oxoethyl)piperazin-1-yl)propoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(3-(4-(2-(4-(3-(2,6-dioxopiperidin-3-yl)-1-methyl-1H-indazol-6-yl)piperazin-1-yl)-2-oxoethyl)piperazin-1-yl)propoxy)-2-methylphenyl)picolinate (110 mg, 101.3 μmol, 1.00 equiv.) in DCM (1 mL) was added trifluoroacetic acid (1.99 g, 17.48 mmol, 1.00 mL, 172.61 equiv.). The reaction solution was stirred at 40° C. After 1 h, the reaction mixture was concentrated and purified by prep-HPLC (column: Phenomenex luna C18 150*25 mm*10 um; mobile phase: [water (1% formic acid)-ACN]; B %: 22%-52%, 10 min) to give 6-(8-(benzo[d]thiazol-2-ylcarbamoyl)-3,4-dihydroisoquinolin-2(1H)-yl)-3-(3-(3-(4-(2-(4-(3-(2,6-dioxopiperidin-3-yl)-1-methyl-1H-indazol-6-yl)piperazin-1-yl)-2-oxoethyl)piperazin-1-yl)propoxy)-2-methylphenyl)picolinic acid (22.1 mg, 20.0 μmol, 19.7% yield, 97.3% purity) as a white solid. MS (ESI) m/z: 1030.7 [M+H]⁺; ¹H NMR (400 MHz, DMSO-d₆) δ=10.84 (s, 1H), 8.16 (s, 1H), 8.03 (d, J=7.6 Hz, 1H), 7.79 (d, J=8.0 Hz, 1H), 7.63 (m, 1H), 7.53 (d, J=8.8 Hz, 1H), 7.49-7.33 (m, 6H), 7.11-7.07 (m, 1H), 6.95 (d, J=8.8 Hz, 2H), 6.90-6.86 (m, 2H), 6.64 (d, J=7.6 Hz, 1H), 4.98 (s, 2H), 4.27 (d, J=9.2 Hz, 1H), 3.98 (t, J=5.6 Hz, 2H), 3.94-3.90 (m, 2H), 3.90 (s, 3H), 3.75-3.73 (m, 2H), 3.63-3.61 (m, 2H), 3.28-3.26 (m, 4H), 3.20-3.18 (m, 4H), 3.03 (t, J=5.6 Hz, 2H), 2.62 (t, J=5.6 Hz, 2H), 2.48-2.44 (m, 8H), 2.34-2.30 (m, 1H), 2.16 (d, J=13.2 Hz, 1H), 1.89 (s, 3H), 1.88-1.86 (m, 2H).

### Example 16. Preparation of Compound 220

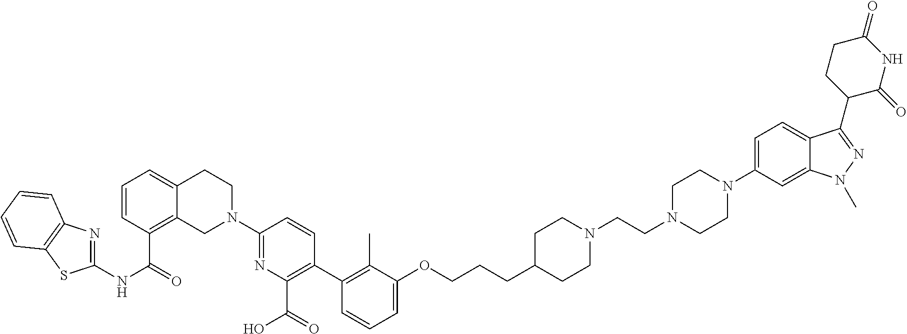 [SMILES: Cc1c(OCCCC2CCN(CCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[1-[2-[4-[3-(2,6-dioxo-3-piperidyl)-1-methyl-indazol-6-yl]piperazin-1-yl]ethyl]-4-piperidyl]propoxy]-2-methyl-phenyl]pyridine-2-carboxylic acid

### Step A. tert-Butyl 4-[3-(3-bromo-2-methyl-phenoxy)propyl]piperidine-1-carboxylate

To a solution of 3-bromo-2-methyl-phenol (1 g, 5.35 mmol, 1 eq.) and tert-butyl 4-(3-bromopropyl)piperidine-1-carboxylate (1.64 g, 5.35 mmol, 1 equiv.) in MeCN (4 mL) was added K₂CO₃ (2.22 g, 16.04 mmol, 3 eq.). The mixture was stirred at 60° C. After 2 h, the reaction solution was concentrated under reduced pressure purified by column chromatography (SiO₂, petroleum ether/ethyl acetate=1/0 to 10/1) to give tert-butyl 4-[3-(3-bromo-2-methyl-phenoxy)propyl]piperidine-1-carboxylate (2.2 g, 5.34 mmol, 99.8% yield) as a colorless oil. MS(ESI) m/z: 312.4 [M+H]⁺; ¹H NMR (400 MHz, CDCl₃) δ=7.14 (d, J=8.0 Hz, 1H), 6.99 (t, J=8.0 Hz, 1H), 6.76 (d, J=8.4 Hz, 1H), 4.14-4.06 (m, 2H), 3.94 (t, J=6.4 Hz, 2H), 2.69 (t, J=2.4, 12.8 Hz, 2H), 2.32 (s, 3H), 1.88-1.79 (m, 2H), 1.70 (d, J=12.8 Hz, 2H), 1.48-1.43 (m, 12H), 1.18-1.08 (m, 2H)

### Step B. 4-[3-(3-bromo-2-methyl-phenoxy)propyl]piperidine

To a solution of tert-butyl 4-[3-(3-bromo-2-methyl-phenoxy)propyl]piperidine-1-carboxylate (2.2 g, 5.34 mmol, 1 equiv.) in 4 M HCl in EtOAc (10 mL) was stirred at 25° C. After 1 h, the reaction mixture was filtered and concentrated under reduced pressure to give 4-[3-(3-bromo-2-methyl-phenoxy)propyl]piperidine (1.6 g, 5.12 mmol, 96.05% yield) as a white solid which was carried forward without further purification. MS(ESI) m/z: 314.4 [M+H]⁺; ¹HNMR (400 MHz, CDCl₃) δ=7.14 (d, J=8.0 Hz, 1H), 6.99 (t, J=8.0 Hz, 1H), 6.76 (d, J=8.4 Hz, 1H), 4.14-4.06 (m, 2H), 3.94 (t, J=6.4 Hz, 2H), 2.69 (t, J=2.4, 12.8 Hz, 2H), 2.32 (s, 3H), 1.88-1.79 (m, 2H), 1.70 (d, J=12.8 Hz, 2H), 1.48-1.43 (m, 12H), 1.18-1.08 (m, 2H).

### Step C. 4-[3-(3-bromo-2-methyl-phenoxy)propyl]-1-(2,2-diethoxyethyl)piperidine

To a solution of 4-[3-(3-bromo-2-methyl-phenoxy)propyl]piperidine (1.5 g, 4.80 mmol, 1 equiv.) and 2-bromo-1,1-diethoxy-ethane (946.71 mg, 4.80 mmol, 722.68 uL, 1 equiv.) in MeCN (5 mL) was added K₂CO₃ (1.99 g, 14.41 mmol, 3 equiv.) and potassium iodide (797.46 mg, 4.80 mmol, 1 equiv.). The mixture was stirred at 80° C. for 8 hours. The reaction mixture was concentrated under reduced pressure and purified by column chromatography (SiO₂, Ethyl acetate/MeOH=1/0 to 20/1) to give 4-[3-(3-bromo-2-methyl-phenoxy)propyl]-1-(2,2-diethoxyethyl)piperidine (1.7 g, 3.97 mmol, 82.6% yield) as a yellow oil. MS(ESI) m/z: 430.5 [M+H]⁺.

### Step D. tert-butyl 6-[8-(1,3-benzothiazol-2-ylcarbamoyl)-3,4-dihydro-1H-isoquinolin-2-yl]-3-[3-[3-[1-(2,2-diethoxyethyl)-4-piperidyl]propoxy]-2-methyl-phenyl]pyridine-2-carboxylate

A mixture of 4-[3-(3-bromo-2-methyl-phenoxy)propyl]-1-(2,2-diethoxyethyl)piperidine (800 mg, 1.87 mmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (1.14 g, 1.87 mmol, 1 equiv.), [2-(2-aminophenyl)phenyl]palladium(1+); bis(1-adamantyl)-butyl-phosphane; methanesulfonate (136.00 mg, 186.74 umol, 0.1 eq.), and KF (1.5 M, 3.73 mL, 3 equiv.) in dioxane (5 mL) and H₂O (1 mL) was degassed and purged with N₂ three times. The reaction mixture was then stirred at 100° C. in a microwave reactor. After 1 h, the reaction solution was concentrated under reduced pressure and purified by column chromatography (SiO₂, ethyl acetate/MeOH=1/0 to 10/1) to give tert-butyl 6-[8-(1,3-benzothiazol-2-ylcarbamoyl)-3,4-dihydro-1H-isoquinolin-2-yl]-3-[3-[3-[1-(2,2-diethoxyethyl)-4-piperidyl]propoxy]-2-methyl-phenyl]pyridine-2-carboxylate (1.5 g, 1.72 mmol, 91.99% yield, 95.52% purity) as a yellow oil, which was carried forward without further purification. MS(ESI) m/z: 834.8 [M+H]⁺; ¹H NMR (400 MHz, CDCl₃) δ=7.85 (d, J=8.0 Hz, 1H), 7.59 (dd, J=7.2, 15.6 Hz, 2H), 7.40-7.30 (m, 5H), 7.14-7.06 (m, 1H), 6.90 (d, J=8.8 Hz, 1H), 6.80 (d, J=8.4 Hz, 1H), 6.69 (d, J=7.6 Hz, 1H), 5.12-4.95 (m, 2H), 4.81-4.60 (m, 1H), 4.12-4.08 (m, 2H), 3.97 (t, J=6.4 Hz, 2H), 3.72-3.68 (m, 2H), 3.61-3.54 (m, 2H), 3.07 (t, J=5.2 Hz, 4H), 2.64-2.52 (m, 2H), 2.05 (s, 3H), 1.84-1.80 (m, 2H), 1.69-1.55 (m, 9H), 1.24-1.20 (m, 6H), 1.15 (s, 9H).

### Step E. 6-[8-(1,3-benzothiazol-2-ylcarbamoyl)-3,4-dihydro-1H-isoquinolin-2-yl]-3-[2-methyl-3-[3-[1-(2-oxoethyl)-4-piperidyl]propoxy]phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[3-[1-(2,2-diethoxyethyl)-4-piperidyl]propoxy]-2-methyl-phenyl]pyridine-2-carboxylate (100 mg, 119.89 umol, 1 equiv.) in formic acid (2 mL) was stirred at 90° C. for 2 hours. The reaction mixture was concentrated under reduced pressure to give 6-[8-(1,3-benzothiazol-2-ylcarbamoyl)-3,4-dihydro-1H-isoquinolin-2-yl]-3-[2-methyl-3-[3-[1-(2-oxoethyl)-4-piperidyl]propoxy]phenyl]pyridine-2-carboxylic acid hydrochloride (70 mg, 99.5 umol, 83% yield) as a dark green oil, which was carried forward without further purification. MS(ESI) m/z: 722.5 [M+H]⁺.

### Step F. 6-[8-(1,3-benzothiazol-2-ylcarbamoyl)-3,4-dihydro-1H-isoquinolin-2-yl]-3-[3-[3-[1-[2-[4-[3-(2,6-dioxo-3-piperidyl)-1-methyl-indazol-6-yl]piperazin-1-yl]ethyl]-4-piperidyl]propoxy]-2-methyl-phenyl]pyridine-2-carboxylic acid

To a solution of 3-(1-methyl-6-piperazin-1-yl-indazol-3-yl)piperidine-2,6-dione (83.72 mg, 255.74 umol, 0.9 eq.) and NaBH(OAc)₃ (180.67 mg, 852.46 umol, 3.0 eq.) in isopropanol (1.5 mL) and DCM (1.5 mL) was added 6-[8-(1,3-benzothiazol-2-ylcarbamoyl)-3,4-dihydro-1H-isoquinolin-2-yl]-3-[2-methyl-3-[3-[1-(2-oxoethyl)-4-piperidyl]propoxy]phenyl]pyridine-2-carboxylic acid (200 mg, 284.15 umol, 1.0 eq.) at 0° C. slowly. The mixture was stirred at 0° C. for 1 hour, then the mixture was warmed to 25° C. and stirred at 25° C. for 12 hours. The mixture was concentrated under reduced pressure, diluted with N,N-dimethylformamide (2 mL) and purified by prep-HPLC (column: Phenomenex luna C18 150×25 mm×10 um; mobile phase: [water (1% formic acid)-acetonitrile]; B %: 20%-50%, 8 min) to give 6-[8-(1,3-benzothiazol-2-ylcarbamoyl)-3,4-dihydro-1H-isoquinolin-2-yl]-3-[3-[3-[1-[2-[4-[3-(2,6-dioxo-3-piperidyl)-1-methyl-indazol-6-yl]piperazin-1-yl]ethyl]-4-piperidyl]propoxy]-2-methyl-phenyl]pyridine-2-carboxylic acid (34.9 mg, 32.8 umol, 11.5% yield, 99.6% purity) as a white solid. MS (ESI) m/z: 1016.8 [M+H]⁺; ¹H NMR (400 MHz, DMSO-d₆) δ=10.85 (s, 1H), 8.01 (d, J=7.6 Hz, 1H), 7.78 (d, J=8.0 Hz, 1H), 7.64-7.59 (m, 1H), 7.54-7.26 (m, 6H), 7.10-7.03 (m, 1H), 6.93-6.89 (m, 1H), 6.88-6.78 (m, 3H), 6.66 (d, J=7.6 Hz, 1H), 4.96 (s, 2H), 4.25 (dd, J=5.2, 9.2 Hz, 1H), 3.95 (s, 4H), 3.91-3.87 (m, 5H), 3.19 (s, 4H), 3.03-3.95 (m, 4H), 2.65-2.58 (m, 4H), 2.58-2.54 (m, 4H), 2.34-2.26 (m, 1H), 2.22-2.10 (m, 3H), 1.90 (s, 3H), 1.77-1.66 (m, 2H), 1.65-1.52 (m, 2H), 1.37-1.22 (m, 3H), 1.21-0.94 (m, 2H).

### Example 17. Preparation of Compound 117a

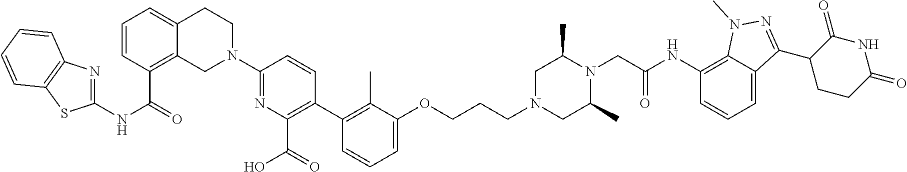 [SMILES: Cc1c(OCCCN2C[C@@H](C)N(CC(=O)Nc3cccc4c(C5CCC(=O)NC5=O)nn(C)c34)[C@@H](C)C2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[(3S,5R)-4-[2-[[3-(2,6-dioxo-3-piperidyl)-1-methyl-indazol-7-yl]amino]-2-oxo-ethyl]-3,5-dimethyl-piperazin-1-yl]propoxy]-2-methyl-phenyl]pyridine-2-carboxylic acid

### Step A. Procedure for Preparation of tert-butyl 6-[8-(1,3-benzothiazol-2-ylcarbamoyl)-3,4-dihydro-1H-isoquinolin-2-yl]-3-[3-[3-[(3S,5R)-4-[2-[[3-(2,6-dioxo-3-piperidyl)-1-methyl-indazol-7-yl]amino]-2-oxo-ethyl]-3,5-dimethyl-piperazin-1-yl]propoxy]-2-methyl-phenyl]pyridine-2-carboxylate

A mixture of 2-[(2S,6R)-4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-2,6-dimethyl-piperazin-1-yl]acetic acid (100 mg, 124.22 μmol, 1 equiv.), 3-(7-amino-1-methyl-indazol-3-yl)piperidine-2,6-dione (32.08 mg, 124.22 μmol, 1 equiv.), HATU (47.23 mg, 124.22 μmol, 1 equiv.), DIEA (16.06 mg, 124.22 μmol, 21.64 μL, 1 equiv.) in DMF (2 mL), and then the mixture was stirred at 60° C. for 12 hours. H₂O (20 mL) was then added dropwise into the reaction mixture, which was filtered to give compound tert-butyl 6-[8-(1,3-benzothiazol-2-ylcarbamoyl)-3,4-dihydro-1H-isoquinolin-2-yl]-3-[3-[3-[(3S,5R)-4-[2-[[3-(2,6-dioxo-3-piperidyl)-1-methyl-indazol-7-yl]amino]-2-oxo-ethyl]-3,5-dimethyl-piperazin-1-yl]propoxy]-2-methyl-phenyl]pyridine-2-carboxylate (90 mg, 86.1 μmol, 69.3% yield) as a yellow solid.

MS (ESI) m/z: 523.4 [M12+H]⁺

### Step B. Procedure for Preparation of 6-[8-(1,3-benzothiazol-2-ylcarbamoyl)-3,4-dihydro-1H-isoquinolin-2-yl]-3-[3-[3-[(3S,5R)-4-[2-[[3-(2,6-dioxo-3-piperidyl)-1-methyl-indazol-7-yl]amino]-2-oxo-ethyl]-3,5-dimethyl-piperazin-1-yl]propoxy]-2-methyl-phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[3-[3-[(3S,5R)-4-[2-[[3-(2,6-dioxo-3-piperidyl)-1-methyl-indazol-7-yl]amino]-2-oxo-ethyl]-3,5-dimethyl-piperazin-1-yl]propoxy]-2-methyl-phenyl]pyridine-2-carboxylate (90 mg, 86.10 μmol, 1 equiv.) in DCM (1 mL) and TFA (1 mL) was stirred at 40° C. for 4 hours. The reaction mixture was concentrated under reduced pressure to remove solvent to give the crude product. The crude product was purified by reverse-phase HPLC to give compound 6-[8-(1,3-benzothiazol-2-ylcarbamoyl)-3,4-dihydro-1H-isoquinolin-2-yl]-3-[3-[3-[(3S,5R)-4-[2-[[3-(2,6-dioxo-3-piperidyl)-1-methyl-indazol-7-yl]amino]-2-oxo-ethyl]-3,5-dimethyl-piperazin-1-yl]propoxy]-2-methyl-phenyl]pyridine-2-carboxylic acid (20 mg, 17.8 μmol, 20.6% yield, 88% purity) as a white solid.

MS (ESI) m/z: 989.3 [M+H]⁺

¹H NMR (400 MHz, DMSO-d₆) δ=10.96 (s, 1H), 9.87 (s, 1H), 8.21 (s, 1H), 8.09 (d, J=8.0 Hz, 1H), 7.85 (d, J=8.0 Hz, 1H), 7.68 (d, J=8.4 Hz, 1H), 7.63 (d, J=8.0 Hz, 1H), 7.58-7.34 (m, 7H), 7.20-7.09 (m, 2H), 7.02 (d, J=8.8 Hz, 1H), 6.94 (d, J=8.4 Hz, 1H), 6.70 (d, J=7.6 Hz, 1H), 5.04 (s, 2H), 4.44 (dd, J=5.2, 10.0 Hz, 1H), 4.18 (s, 3H), 4.06 (t, J=5.6 Hz, 2H), 3.98 (t, J=5.6 Hz, 2H), 3.65 (s, 1H), 3.09 (t, J=5.6 Hz, 2H), 2.87 (d, J=10. Hz, 2H), 2.81-2.69 (m, 4H), 2.53-2.36 (m, 4H), 2.22 (dd, J=5.2, 13.2 Hz, 1H), 2.00-1.90 (m, 7H), 1.15 (d, J=6.0 Hz, 6H).

### Example 18. Preparation of Compound 116a

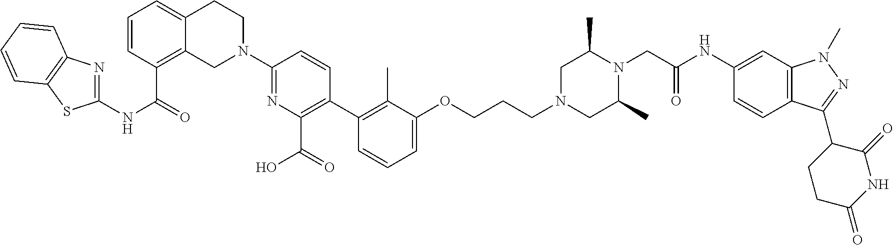 [SMILES: Cc1c(OCCCN2C[C@@H](C)N(CC(=O)Nc3ccc4c(C5CCC(=O)NC5=O)nn(C)c4c3)[C@@H](C)C2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[(3S,5R)-4-[2-[[3-(2,6-dioxo-3-piperidyl)-1-methyl-indazol-6-yl]amino]-2-oxo-ethyl]-3,5-dimethyl-piperazin-1-yl]propoxy]-2-methyl-phenyl]pyridine-2-carboxylic acid

### Step A. Procedure for Preparation of 1-bromo-3-(3-bromopropoxy)-2-methyl-benzene

To a solution of 3-bromo-2-methyl-phenol (2 g, 10.69 mmol, 1 equiv.) in acetone (30 mL) were added 1,3-dibromopropane (5.18 g, 25.66 mmol, 2.62 mL, 2.4 equiv.) and K₂CO₃ (4.43 g, 32.08 mmol, 3 equiv.), the mixture was stirred at 60° C. for 12 hours. The reaction mixture was concentrated under reduced pressure to give the residue. The residue was purified by flash silica gel chromatography (eluent of 0-5% ethyl acetate/petroleum ether) to give the compound 1-bromo-3-(3-bromopropoxy)-2-methyl-benzene (1.6 g, 5.2 mmol, 48.6% yield) as a colorless oil.

¹H NMR (400 MHz, CDCl₃) δ=7.09 (d, J=8.0 Hz, 1H), 6.93 (t, J=8.0 Hz, 1H), 6.71 (d, J=8.4 Hz, 1H), 4.02 (t, J=5.6 Hz, 2H), 3.54 (t, J=6.4 Hz, 2H), 2.27 (t, J=6.0 Hz, 2H), 2.24 (s, 3H)

### Step B. Procedure for Preparation of (3S,5R)-1-[3-(3-bromo-2-methyl-phenoxy)propyl]-3,5-dimethyl-piperazine

A mixture of 1-bromo-3-(3-bromopropoxy)-2-methyl-benzene (1.6 g, 5.19 mmol, 1 equiv.), (2S, 6R)-2,6-dimethylpiperazine (593.17 mg, 5.19 mmol, 1 equiv.), K₂CO₃ (1.44 g, 10.39 mmol, 2 equiv.) in CH₃CN (20 mL) was stirred at 60° C. for 12 hours. The reaction mixture was filtered and concentrated under reduced pressure to give compound (3S,5R)-1-[3-(3-bromo-2-methyl-phenoxy)propyl]-3,5-dimethyl-piperazine (1.75 g, 5.1 mmol, 98.7% yield) as a yellow oil.

MS (ESI) m/z: 341.1 [M+H]⁺

¹H NMR (400 MHz, CDCl₃) δ=7.06 (d, J=7.6 Hz, 1H), 6.91 (t, J=8.0 Hz, 1H), 6.70 (d, J=8.0 Hz, 1H), 3.93 (t, J=6.4 Hz, 2H), 2.86-2.84 (m, 2H), 2.74 (dd, J=2.4, 11.6 Hz, 2H), 2.47-2.42 (m, 2H), 2.24 (s, 3H), 1.95-1.90 (m, 2H), 1.53 (t, J=10.8 Hz, 2H), 0.99 (d, J=6.4 Hz, 6H).

### Step C. Procedure for Preparation of ethyl 2-[(2S,6R)-4-[3-(3-bromo-2-methyl-phenoxy)propyl]-2,6-dimethyl-piperazin-1-yl]acetate

A mixture of (3S,5R)-1-[3-(3-bromo-2-methyl-phenoxy)propyl]-3,5-dimethyl-piperazine (1.75 g, 5.13 mmol, 1 equiv.), ethyl 2-bromoacetate (856.33 mg, 5.13 mmol, 567.10 μL, 1 equiv.), K₂CO₃ (1.42 g, 10.26 mmol, 2.0 equiv.), KI (851.20 mg, 5.13 mmol, 1 equiv.) in CH₃CN (20 mL) was stirred at 50° C. for 16 hours. The reaction mixture was concentrated under reduced pressure to give a residue. The residue was purified by column chromatography (DCM:MeOH=1/0 to 10/1) to give compound ethyl 2-[(2S,6R)-4-[3-(3-bromo-2-methyl-phenoxy)propyl]-2,6-dimethyl-piperazin-1-yl]acetate (2.0 g, 4.7 mmol, 91.3% yield) as a yellow oil.

MS (ESI) m/z: 429.0 [M+H]⁺

### Step D. Procedure for Preparation of tert-butyl 6-[8-(1,3-benzothiazol-2-ylcarbamoyl)-3,4-dihydro-1H-isoquinolin-2-yl]-3-[3-[3-[(3S,5R)-4-(2-ethoxy-2-oxo-ethyl)-3,5-dimethyl-piperazin-1-yl]propoxy]-2-methyl-phenyl]pyridine-2-carboxylate

Ethyl 2-[(2S,6R)-4-[3-(3-bromo-2-methyl-phenoxy)propyl]-2,6-dimethyl-piperazin-1-yl]acetate (200 mg, 467.97 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (286.66 mg, 467.97 μmol, 1 equiv.), K₂CO₃ (129.36 mg, 935.95 μmol, 2.0 equiv.) and Ad₂nBuP Pd G₃(cataCXium® A Pd G₃) (34.08 mg, 46.80 μmol, 0.1 equiv.) were taken up into a microwave tube in dioxane (5 mL) and H₂O (5 mL). The sealed tube was heated at 100° C. for 2 hours under microwave. The reaction mixture was concentrated under reduced pressure to remove solvent to give the crude product. The crude product was purified by reverse-phase HPLC to give compound tert-butyl 6-[8-(1,3-benzothiazol-2-ylcarbamoyl)-3,4-dihydro-1H-isoquinolin-2-yl]-3-[3-[3-[(3S,5R)-4-(2-ethoxy-2-oxo-ethyl)-3,5-dimethyl-piperazin-1-yl]propoxy]-2-methyl-phenyl]pyridine-2-carboxylate (172 mg, 206.4 μmol, 44.1% yield) as a yellow oil.

MS (ESI) m/z: 833.5 [M+H]⁺

### Step E. Procedure for Preparation of 2-[(2S,6R)-4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-2,6-dimethyl-piperazin-1-yl]acetic acid

A mixture of tert-butyl 6-[8-(1,3-benzothiazol-2-ylcarbamoyl)-3,4-dihydro-1H-isoquinolin-2-yl]-3-[3-[3-[(3S,5R)-4-(2-ethoxy-2-oxo-ethyl)-3,5-dimethyl-piperazin-1-yl]propoxy]-2-methyl-phenyl]pyridine-2-carboxylate (270 mg, 324.11 μmol, 1 equiv.), LiOH·H₂O (81.61 mg, 1.94 mmol, 6.0 equiv.) in THE (5 mL) and H₂O (5 mL) was stirred at 20° C. for 12 hours. The reaction mixture was concentrated under reduced pressure to remove THF. The mixture was adjusted pH to 2-3 and then extracted with DCM (10 mL×3). The combined organic layers were concentrated under reduced pressure to give compound 2-[(2S,6R)-4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-2,6-dimethyl-piperazin-1-yl]acetic acid (260 mg, 322.9 μmol, 99.6% yield) as a yellow solid.

MS (ESI) m/z: 805.4[M+H]⁺

### Step F. Procedure for Preparation of tert-butyl 6-[8-(1,3-benzothiazol-2-ylcarbamoyl)-3,4-dihydro-1H-isoquinolin-2-yl]-3-[3-[3-[(3S,5R)-4-[2-[[3-(2,6-dioxo-3-piperidyl)-1-methyl-indazol-6-yl]amino]-2-oxo-ethyl]-3,5-dimethyl-piperazin-1-yl]propoxy]-2-methyl-phenyl]pyridine-2-carboxylate

A mixture of 2-[(2S,6R)-4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-2,6-dimethyl-piperazin-1-yl]acetic acid (100 mg, 124.22 μmol, 1 equiv.), 3-(6-amino-1-methyl-indazol-3-yl)piperidine-2,6-dione (32.08 mg, 124.22 μmol, 1 equiv.), HATU (47.23 mg, 124.22 μmol, 1 equiv.), DIEA (16.06 mg, 124.22 μmol, 21.64 μL, 1.0 equiv.) in DMF (3 mL) was stirred at 25° C. for 2 hours. H₂O (20 mL) was added dropwise into the reaction mixture. The mixture was then filtered and concentrated under reduced pressure to give compound tert-butyl 6-[8-(1,3-benzothiazol-2-ylcarbamoyl)-3,4-dihydro-1H-isoquinolin-2-yl]-3-[3-[3-[(3S,5R)-4-[2-[[3-(2,6-dioxo-3-piperidyl)-1-methyl-indazol-6-yl]amino]-2-oxo-ethyl]-3,5-dimethyl-piperazin-1-yl]propoxy]-2-methyl-phenyl]pyridine-2-carboxylate (110 mg, 105.2 μmol, 84.7% yield) as a pink solid.

MS (ESI) m/z: 523.3.3[M/2+H]⁺

### Step G. Procedure for Preparation of 6-[8-(1,3-benzothiazol-2-ylcarbamoyl)-3,4-dihydro-1H-isoquinolin-2-yl]-3-[3-[3-[(3S,5R)-4-[2-[[3-(2,6-dioxo-3-piperidyl)-1-methyl-indazol-6-yl]amino]-2-oxo-ethyl]-3,5-dimethyl-piperazin-1-yl]propoxy]-2-methyl-phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[3-[3-[(3S,5R)-4-[2-[[3-(2,6-dioxo-3-piperidyl)-1-methyl-indazol-6-yl]amino]-2-oxo-ethyl]-3,5-dimethyl-piperazin-1-yl]propoxy]-2-methyl-phenyl]pyridine-2-carboxylate (110 mg, 105.24 μmol, 1 equiv.) in TFA (1 mL) and DCM (1 mL), the mixture was stirred at 40° C. for 4 hours. The reaction mixture was concentrated under reduced pressure to give a residue. The residue was purified by prep-HPLC to give compound 6-[8-(1,3-benzothiazol-2-ylcarbamoyl)-3,4-dihydro-1H-isoquinolin-2-yl]-3-[3-[3-[(3S,5R)-4-[2-[[3-(2,6-dioxo-3-piperidyl)-1-methyl-indazol-6-yl]amino]-2-oxo-ethyl]-3,5-dimethyl-piperazin-1-yl]propoxy]-2-methyl-phenyl]pyridine-2-carboxylic acid (50.3 mg, 49.2 μmol, 46.8% yield, 96.8% purity) as a white solid.

MS (ESI) m/z: 989.3 [M+H]⁺

¹H NMR (400 MHz, DMSO-d₆) δ=10.89 (s, 1H), 9.78 (s, 1H), 8.15 (s, 1H), 8.05-8.02 (m, 2H), 7.79 (d, J=8.4 Hz, 1H), 7.67-7.60 (m, 2H), 7.48-7.34 (m, 5H), 7.24 (d, J=8.8 Hz, 1H), 7.14-7.06 (m, 1H), 6.98 (d, J=8.8 Hz, 1H), 6.89 (d, J=8.4 Hz, 1H), 6.65 (d, J=7.6 Hz, 1H), 4.99 (s, 2H), 4.34 (dd, J=5.2, 9.6 Hz, 1H), 4.01 (t, J=5.2 Hz, 2H), 3.98-3.85 (m, 6H), 3.03 (t, J=5.2 Hz, 2H), 2.85-2.75 (m, 4H), 2.70-2.56 (m, 3H), 2.40-2.29 (m, 1H), 2.21-2.13 (m, 1H), 2.42-2.12 (m, 1H), 1.95-1.87 (m, 7H), 1.02 (d, J=5.6 Hz, 6H).

### Example 19. Preparation of Compound 119

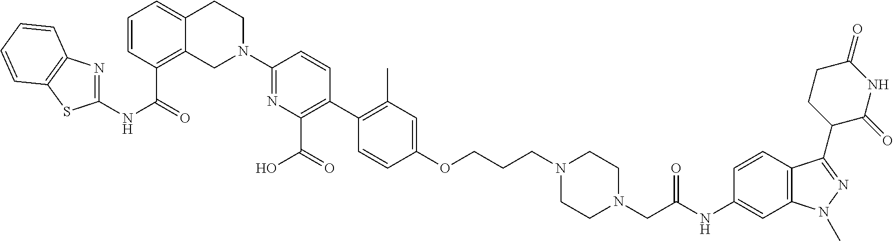 [SMILES: Cc1cc(OCCCN2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[4-[3-[4-[2-[[3-(2,6-dioxo-3-piperidyl)-1-methyl-indazol-6-yl]amino]-2-oxo-ethyl]piperazin-1-yl]propoxy]-2-methyl-phenyl]pyridine-2-carboxylic acid

### Step A. Procedure for Preparation of tert-butyl 4-[3-(4-bromo-3-methyl-phenoxy) propyl]piperazine-1-carboxylate

A mixture of 4-bromo-3-methyl-phenol (382.75 mg, 2.05 mmol, 1 equiv.), tert-butyl 4-(3-hydroxypropyl)piperazine-1-carboxylate (500 mg, 2.05 mmol, 1 equiv.), PPh₃ (697.77 mg, 2.66 mmol, 1.3 equiv.) in THF (9 mL) was degassed and purged with N₂ three times, and then DIAD (496.56 mg, 2.46 mmol, 477.46 μL, 1.2 equiv.) was added dropwise at 0° C. The mixture was stirred at 25° C. for 12 hours under N₂ atmosphere. The reaction mixture was diluted with H₂O (5 mL×3) and extracted with ethyl acetate (10 mL×3). The combined organic layers were washed with brine (5 mL×3), dried over Na₂SO₄, filtered, and concentrated under reduced pressure to give a residue. The residue was purified by flash silica gel chromatography (eluent of 0˜47% ethyl acetate/petroleum ether) to give compound tert-butyl 4-[3-(4-bromo-3-methyl-phenoxy) propyl]piperazine-1-carboxylate (450 mg, 870.9 μmol, 42.5% yield, 80% purity) as a yellow oil.

MS (ESI) m/z: 414.9 [M+H]⁺.

¹H NMR (400 MHz, CD₃OD) δ=7.38 (d, J=8.4 Hz, 1H), 6.85 (d, J=3.2 Hz, 1H), 6.66 (dd, J=3.2, 3.2 Hz, 1H), 4.00 (t, J=6.4 Hz, 2H), 3.43 (d, J=4.4 Hz, 4H), 2.55 (t, J=7.2 Hz, 2H), 2.48-2.40 (m, 4H), 2.33 (s, 3H), 2.00-1.92 (m, 2H), 1.46 (s, 9H).

### Step B. Procedure for Preparation of 1-[3-(4-bromo-3-methyl-phenoxy) propyl] piperazine

To a solution of tert-butyl 4-[3-(4-bromo-3-methyl-phenoxy) propyl] piperazine-1-carboxylate (450 mg, 1.09 mmol, 1 equiv.) was added HCl/dioxane (4 M, 4.50 mL, 16.53 equiv.). The mixture was stirred at 25° C. for 1 hour. The reaction mixture was concentrated under reduced pressure to give compound 1-[3-(4-bromo-3-methyl-phenoxy) propyl] piperazine (400 mg, crude) as a white solid.

MS (ESI) m/z: 315.0 [M+H]⁺.

### Step C. Procedure for Preparation of ethyl 2-[4-[3-(4-bromo-3-methyl-phenoxy)propyl]piperazin-1-yl]acetate

To a solution of 1-[3-(4-bromo-3-methyl-phenoxy) propyl] piperazine (350 mg, 1.00 mmol, 1 eq, HCl) in CH₃CN (3 mL) was added K₂CO₃ (414.98 mg, 3.00 mmol, 3 equiv.) and ethyl 2-bromoacetate (167.15 mg, 1.00 mmol, 110.69 μL, 1 equiv.). The mixture was stirred at 25° C. for 1 hour. The reaction mixture was filtered and concentrated under reduced pressure to give the crude product. The residue was purified by prep-TLC (SiO₂, DCM:MeOH=30:1) to give compound ethyl 2-[4-[3-(4-bromo-3-methyl-phenoxy)propyl]piperazin-1-yl]acetate (230 mg, 518.3 μmol, 51.7% yield, 90% purity) as a yellow oil.

MS (ESI) m/z: 401.1 [M+H]⁺

### Step D. Procedure for Preparation of tert-butyl 6-[8-(1,3-benzothiazol-2-ylcarbamoyl)-3,4-dihydro-1H-isoquinolin-2-yl]-3-[4-[3-[4-(2-ethoxy-2-oxo-ethyl)piperazin-1-yl]propoxy]-2-methyl-phenyl]pyridine-2-carboxylate

A mixture of tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (260 mg, 424.46 μmol, 1 equiv.), ethyl 2-[4-[3-(4-bromo-3-methyl-phenoxy)propyl]piperazin-1-yl]acetate (225.99 mg, 509.35 μmol, 90% purity, 1.2 equiv.) in dioxane (3 mL) was added KF (1.5 M, 848.92 μL, 3 equiv.) and Ad₂nBuP Pd G3(cataCXium® A Pd G₃) (30.91 mg, 42.45 μmol, 0.1 equiv.). After addition, the mixture was degassed and purged with N₂ three times, and then the mixture was stirred at 100° C. for 2 hours under N₂ atmosphere. The reaction mixture was diluted with H₂O 6 mL and extracted with ethyl acetate (5 mL×4). The combined organic layers were washed with brine (5 mL×2), dried over Na₂SO₄, filtered, and concentrated under reduced pressure to give a residue. The residue was purified by flash silica gel chromatography (eluent of 0-9% dichloromethane/methanol) to give compound tert-butyl 6-[8-(1,3-benzothiazol-2-ylcarbamoyl)-3,4-dihydro-1H-isoquinolin-2-yl]-3-[4-[3-[4-(2-ethoxy-2-oxo-ethyl)piperazin-1-yl]propoxy]-2-methyl-phenyl]pyridine-2-carboxylate (220 mg, 218.6 μmol, 51.5% yield, 80% purity) as a yellow oil.

MS (ESI) m/z: 805.3 [M+H]⁺

¹H NMR (400 MHz, CDCl₃) δ=7.85 (d, J=7.6 Hz, 1H), 7.56 (dd, J=5.2, 4.0 Hz, 2H), 7.38-7.34 (m, 3H), 7.32-7.29 (m, 1H), 6.96 (d, J=8.4 Hz, 1H), 6.88 (d, J=8.8 Hz, 1H), 6.78 (d, J=2.8 Hz, 1H), 6.70 (dd, J=2.4, 2.4 Hz, 1H), 6.61 (dd, J=3.2, 2.8 Hz, 1H), 4.11-4.07 (m, 2H), 4.03-3.98 (m, 2H), 3.23 (s, 3H), 3.06 (t, J=5.6 Hz, 2H), 2.72-2.57 (m, 12H), 2.36 (s, 2H), 2.09 (s, 3H), 1.69-1.54 (m, 4H), 1.18 (s, 9H)

### Step E. Procedure for Preparation of 2-[4-[3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]propyl]piperazin-1-yl]acetic acid

To a solution of tert-butyl 6-[8-(1,3-benzothiazol-2-ylcarbamoyl)-3,4-dihydro-1H-isoquinolin-2-yl]-3-[4-[3-[4-(2-ethoxy-2-oxo-ethyl)piperazin-1-yl]propoxy]-2-methyl-phenyl]pyridine-2-carboxylate (220 mg, 218.64 μmol, 80% purity, 1 equiv.) in THE (2 mL) was added LiOH (15.71 mg, 655.91 μmol, 3 equiv.) and H₂O (0.5 mL) at 25° C. The mixture was stirred at 25° C. for 12 hours. The reaction mixture was concentrated under reduced pressure to remove THF. The aqueous phase was adjusted to pH=4˜5 with 1 M HCl. The reaction mixture was filtered. The solid was diluted in ethyl acetate. The combined organic layers were dried over Na₂SO₄, filtered and concentrated under reduced pressure to give compound 2-[4-[3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]propyl]piperazin-1-yl]acetic acid (160 mg, crude) as a yellow oil.

MS (ESI) m/z: 777.2 [M+H]⁺

### Step F. Procedure for Preparation of tert-butyl 6-[8-(1,3-benzothiazol-2-ylcarbamoyl)-3,4-dihydro-1H-isoquinolin-2-yl]-3-[4-[3-[4-[2-[[3-(2,6-dioxo-3-piperidyl)-1-methyl-indazol-6-yl]amino]-2-oxo-ethyl]piperazin-1-yl]propoxy]-2-methyl-phenyl]pyridine-2-carboxylate

To a solution of 2-[4-[3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]propyl]piperazin-1-yl]acetic acid (80 mg, 102.97 μmol, 1 equiv.) in DMF (1 mL) was added HATU (46.98 mg, 123.56 μmol, 1.2 equiv.), DIPEA (39.92 mg, 308.90 μmol, 53.80 μL, 3 equiv.), and 3-(6-amino-1-methyl-indazol-3-yl)piperidine-2,6-dione (31.91 mg, 123.56 μmol, 1.2 equiv.). The mixture was stirred at 25° C. for 3 hours. The reaction mixture was quenched by addition H₂O (2 mL). The reaction mixture was filtered and washed with water (5 mL). The solid was diluted in DCM (10 mL). The combined organic layers were dried over Na₂SO₄, filtered, and concentrated under reduced pressure to give tert-butyl 6-[8-(1,3-benzothiazol-2-ylcarbamoyl)-3,4-dihydro-1H-isoquinolin-2-yl]-3-[4-[3-[4-[2-[[3-(2,6-dioxo-3-piperidyl)-1-methyl-indazol-6-yl]amino]-2-oxo-ethyl]piperazin-1-yl]propoxy]-2-methyl-phenyl]pyridine-2-carboxylate (90 mg, crude) as a yellow solid.

MS (ESI) m/z: 1017.4 [M+H]⁺

### Step G. Procedure for Preparation of 6-[8-(1,3-benzothiazol-2-ylcarbamoyl)-3,4-dihydro-1H-isoquinolin-2-yl]-3-[4-[3-[4-[2-[[3-(2,6-dioxo-3-piperidyl)-1-methyl-indazol-6-yl]amino]-2-oxo-ethyl]piperazin-1-yl]propoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[4-[3-[4-[2-[[3-(2,6-dioxo-3-piperidyl)-1-methyl-indazol-6-yl]amino]-2-oxo-ethyl]piperazin-1-yl]propoxy]-2-methyl-phenyl]pyridine-2-carboxylate (90 mg, 88.48 μmol, 1 equiv.) in DCM (0.5 mL) was added TFA (770.00 mg, 6.75 mmol, 500.00 μL, 76.32 equiv.). The mixture was stirred at 25° C. for 1 hour. The reaction mixture was concentrated under reduced pressure to give a residue. The residue was purified by prep-HPLC to give compound 6-[8-(1,3-benzothiazol-2-ylcarbamoyl)-3,4-dihydro-1H-isoquinolin-2-yl]-3-[4-[3-[4-[2-[[3-(2,6-dioxo-3-piperidyl)-1-methyl-indazol-6-yl]amino]-2-oxo-ethyl]piperazin-1-yl]propoxy]-2-methyl-phenyl]pyridine-2-carboxylic acid (12.5 mg, 12.2 μmol, 13.8% yield, 94.3% purity) as a yellow solid.

MS (ESI) m/z: 961.1 [M+H]⁺

¹H NMR (400 MHz, DMSO-d₆) δ=10.86 (s, 1H), 9.85 (s, 1H), 8.05-8.01 (m, 2H), 7.79 (d, J=9.2 Hz, 1H), 7.63 (d, J=8.8 Hz, 2H), 7.50-7.33 (m, 6H), 7.22-7.18 (m, 1H), 6.93 (d, J=8.4 Hz, 2H), 6.78 (s, 1H), 6.73-6.68 (m, 1H), 4.97 (s, 2H), 4.36-4.29 (m, 1H), 3.98 (s, 2H), 3.92-3.91 (m, 5H), 3.61-3.56 (m, 2H), 3.16 (s, 3H), 3.05-2.99 (m, 4H), 2.97-2.94 (m, 1H), 2.61 (d, J=6.4 Hz, 4H), 2.18 (s, 3H), 2.03 (s, 4H), 1.89-1.84 (m, 2H).

### Example 20. Preparation of Compound 120

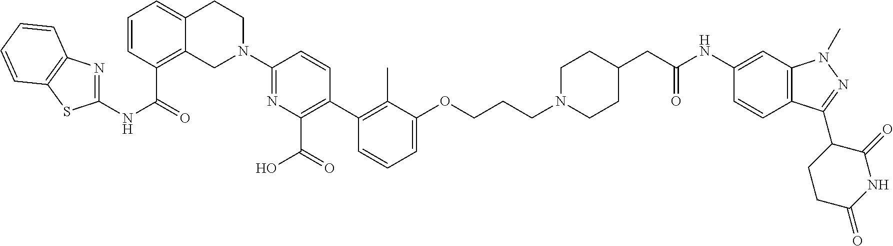 [SMILES: Cc1c(OCCCN2CCC(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[4-[2-[[3-(2,6-dioxo-3-piperidyl)-1-methyl-indazol-6-yl]amino]-2-oxo-ethyl]-1-piperidyl]propoxy]-2-methyl-phenyl]pyridine-2-carboxylic acid

### Step A. Procedure for Preparation of ethyl 2-[1-[3-(3-bromo-2-methyl-phenoxy)propyl]-4-piperidyl]acetate

A mixture of 1-bromo-3-(3-bromopropoxy)-2-methyl-benzene (800 mg, 2.60 mmol, 1 equiv.), ethyl 2-(4-piperidyl)acetate (444.76 mg, 2.60 mmol, 1 equiv.), K₂CO₃ (717.93 mg, 5.19 mmol, 2.0 equiv.) in ACN (20 mL), and then the mixture was stirred at 50° C. for 4 hours. The reaction mixture was concentrated under reduced pressure to remove CH₃CN to give a residue. The residue was purified by column chromatography (SiO₂, DCM:MeOH=1/0 to 10/1) to give compound ethyl 2-[1-[3-(3-bromo-2-methyl-phenoxy)propyl]-4-piperidyl]acetate (650 mg, 1.6 mmol, 62.8% yield) was obtained as a colorless oil.

MS (ESI) m/z: 398.0 [M+H]⁺

¹H NMR (400 MHz, CDCl₃) δ=7.16 (d, J=8.0 Hz, 1H), 7.00 (t, J=8.0 Hz, 1H), 6.79 (d, J=8.4 Hz, 1H), 4.15 (q, J=7.2 Hz, 2H), 4.01 (t, J=6.0 Hz, 2H), 2.95 (d, J=11.6 Hz, 2H), 2.54 (t, J=7.2 Hz, 2H), 2.32 (s, 3H), 2.25 (d, J=7.2 Hz, 2H), 2.05-1.99 (m, 4H), 1.87-1.80 (m, 1H), 1.75 (d, J=14.0 Hz, 2H), 1.41-1.32 (m, 2H), 1.28 (t, J=7.2 Hz, 3H).

### Step B. Procedure for Preparation of tert-butyl 6-[8-(1,3-benzothiazol-2-ylcarbamoyl)-3,4-dihydro-1H-isoquinolin-2-yl]-3-[3-[3-[4-(2-ethoxy-2-oxo-ethyl)-1-piperidyl]propoxy]-2-methyl-phenyl]pyridine-2-carboxylate

Ethyl 2-[1-[3-(3-bromo-2-methyl-phenoxy)propyl]-4-piperidyl]acetate (400 mg, 1.00 mmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (430.58 mg, 702.93 μmol, 0.7 equiv.), K₂CO₃ (277.57 mg, 2.01 mmol, 2.0 equiv.) and Ad₂nBuP Pd G₃(cataCXium® A Pd G₃) (73.13 mg, 100.42 μmol, 0.1 equiv.) were taken up into a microwave tube in dioxane (5 mL) and H₂O (2 mL). The sealed tube was heated at 100° C. for 2 hours under microwave. The reaction mixture was concentrated under reduced pressure to give a residue. The residue was purified by prep-HPLC (neutral condition) to give compound tert-butyl 6-[8-(1,3-benzothiazol-2-ylcarbamoyl)-3,4-dihydro-1H-isoquinolin-2-yl]-3-[3-[3-[4-(2-ethoxy-2-oxo-ethyl)-1-piperidyl]propoxy]-2-methyl-phenyl]pyridine-2-carboxylate (300 mg, 373.1 μmol, 37.1% yield) as a yellow solid.

MS (ESI) m/z: 804.7 [M+H]⁺

### Step C. Procedure for Preparation of 2-[1-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-4-piperidyl]acetic acid

A mixture of tert-butyl 6-[8-(1,3-benzothiazol-2-ylcarbamoyl)-3,4-dihydro-1H-isoquinolin-2-yl]-3-[3-[3-[4-(2-ethoxy-2-oxo-ethyl)-1-piperidyl]propoxy]-2-methyl-phenyl]pyridine-2-carboxylate (300 mg, 373.1 μmol, 1 equiv.), LiOH·H₂O (93.94 mg, 2.24 mmol, 6 equiv.) in THE (6 mL) and H₂O (3 mL) was stirred at 25° C. for 12 hours. The reaction mixture was concentrated under reduced pressure to remove THF, and then adjusted pH to 2˜3 with HCl (3 M). The mixture was then filtered and concentrated under reduced pressure to give compound 2-[1-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-4-piperidyl]acetic acid (280 mg, 360.8 μmol, 96.7% yield) as a yellow solid.

MS (ESI) m/z: 776.2 [M+H]⁺

### Step D. Procedure for Preparation of tert-butyl 6-[8-(1,3-benzothiazol-2-ylcarbamoyl)-3,4-dihydro-1H-isoquinolin-2-yl]-3-[3-[3-[4-[2-[[3-(2,6-dioxo-3-piperidyl)-1-methyl-indazol-6-yl]amino]-2-oxo-ethyl]-1-piperidyl]propoxy]-2-methyl-phenyl]pyridine-2-carboxylate

A mixture of 2-[1-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-4-piperidyl]acetic acid (100 mg, 128.87 μmol, 1 equiv.), 3-(6-amino-1-methyl-indazol-3-yl)piperidine-2,6-dione (33.28 mg, 128.87 μmol, 1 equiv.), HATU (49.00 mg, 128.87 μmol, 1 equiv.), DIEA (16.66 mg, 128.87 μmol, 22.45 μL, 1 equiv.) in DMF (5 mL) was stirred at 25° C. for 2 hours. The reaction mixture was diluted with water 10 mL, filtered to give a the crude product tert-butyl 6-[8-(1,3-benzothiazol-2-ylcarbamoyl)-3,4-dihydro-1H-isoquinolin-2-yl]-3-[3-[3-[4-[2-[[3-(2,6-dioxo-3-piperidyl)-1-methyl-indazol-6-yl]amino]-2-oxo-ethyl]-1-piperidyl]propoxy]-2-methyl-phenyl]pyridine-2-carboxylate (100 mg, 98.4 μmol, 76.4% yield) as a yellow solid.

MS (ESI) m/z: 960.3 [M-56+H]⁺

### Step E. Procedure for Preparation of 6-[8-(1,3-benzothiazol-2-ylcarbamoyl)-3,4-dihydro-1H-isoquinolin-2-yl]-3-[3-[3-[4-[2-[[3-(2,6-dioxo-3-piperidyl)-1-methyl-indazol-6-yl]amino]-2-oxo-ethyl]-1-piperidyl]propoxy]-2-methyl-phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[3-[3-[4-[2-[[3-(2,6-dioxo-3-piperidyl)-1-methyl-indazol-6-yl]amino]-2-oxo-ethyl]-1-piperidyl]propoxy]-2-methyl-phenyl]pyridine-2-carboxylate (100 mg, 98.40 μmol, 1 equiv.) in DCM (1 mL) and TFA (1 mL) was stirred at 40° C. for 12 hours. The reaction mixture was concentrated under reduced pressure to give a crude product. The crude product was purified by reverse-phase HPLC to give compound 6-[8-(1,3-benzothiazol-2-ylcarbamoyl)-3,4-dihydro-1H-isoquinolin-2-yl]-3-[3-[3-[4-[2-[[3-(2,6-dioxo-3-piperidyl)-1-methyl-indazol-6-yl]amino]-2-oxo-ethyl]-1-piperidyl]propoxy]-2-methyl-phenyl]pyridine-2-carboxylic acid (35.1 mg, 34.8 μmol, 35.3% yield, 95% purity) as a white solid.

MS (ESI) m/z: 960.3 [M+H]⁺

¹H NMR (400 MHz, DMSO-d₆) δ=10.86 (s, 1H), 10.08 (s, 1H), 8.18 (s, 1H), 8.09 (s, 1H), 8.03 (d, J=8.0 Hz, 1H), 7.79 (d, J=8.0 Hz, 1H), 7.67-7.59 (m, 2H), 7.46-7.34 (m, 5H), 7.14-7.05 (m, 2H), 6.94 (d, J=8.4 Hz, 1H), 6.87 (d, J=8.4 Hz, 1H), 6.65 (d, J=7.6 Hz, 1H), 4.98 (s, 2H), 4.31 (dd, J=5.2, 9.6 Hz, 1H), 3.99 (t, J=5.6 Hz, 3H), 3.95-3.88 (m, 6H), 3.04 (d, J=5.6 Hz, 2H), 2.93 (d, J=8.4 Hz, 3H), 2.71-2.56 (m, 4H), 2.35-2.32 (m, 1H), 2.29 (d, J=6.4 Hz, 2H), 2.18 (dd, J=5.2, 13.6 Hz, 1H), 2.06 (t, J=11.2 Hz, 2H), 1.91 (s, 3H), 1.69 (d, J=12.4 Hz, 2H), 1.36-1.23 (m, 2H).

### Example 21. Preparation of Compound 127

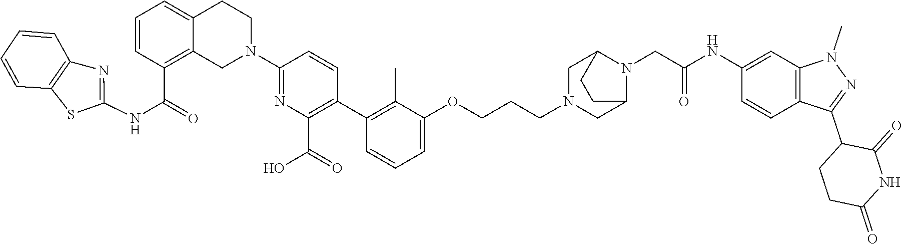 [SMILES: Cc1c(OCCCN2CC3CCC(C2)N3CC(=O)Nc2ccc3c(C4CCC(=O)NC4=O)nn(C)c3c2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-((1R,5S)-8-(2-((3-(2,6-dioxopiperidin-3-yl)-1-methyl-1H-indazol-6-yl)amino)-2-oxoethyl)-3,8-diazabicyclo[3.2.1]octan-3-yl)propoxy)-2-methylphenyl)picolinic acid

### Step A. Procedure for Preparation of (1R, 5S)-tert-butyl 3-(3-(3-bromo-2-methylphenoxy)propyl)-3,8-diazabicyclo[3.2.1]octane-8-carboxylate

To a solution of 1-bromo-3-(3-bromopropoxy)-2-methylbenzene (1 g, 3.25 mmol, 1 equiv.) and (1R,5S)-tert-butyl 3,8-diazabicyclo[3.2.1]octane-8-carboxylate (1.38 g, 6.49 mmol, 2 equiv.) in CH₃CN (10 mL) was added DIPEA (1.26 g, 9.74 mmol, 1.70 mL, 3 equiv.). The mixture was stirred at 60° C. for 12 hours. The mixture was concentrated under reduced pressure to give a residue. The residue was purified by flash silica gel chromatography (eluent of 0-16% ethyl acetate/petroleum ether) to give compound (1R, 5S)-tert-butyl 3-(3-(3-bromo-2-methylphenoxy)propyl)-3,8-diazabicyclo[3.2.1]octane-8-carboxylate (1.5 g, 3.4 mmol, 50.0% yield) as a colorless oil.

MS (ESI) m/z: 441.1 [M+H]⁺.

### Step B. Procedure for Preparation of (1R, 5S)-3-(3-(3-bromo-2-methylphenoxy)propyl)-3,8-diazabicyclo[3.2.1]octane

A mixture of (1R, 5S)-tert-butyl 3-(3-(3-bromo-2-methylphenoxy)propyl)-3,8-diazabicyclo[3.2.1]octane-8-carboxylate (1.5 g, 3.41 mmol, 1 equiv.), HCl/1,4-dioxane (4 M, 10 mL, 11.72 equiv.) in DCM (5 mL) was degassed and purged with N₂ three times, and then the mixture was stirred at 25° C. for 2 hours under N₂ atmosphere. The mixture was concentrated under reduced pressure to give compound (1R, 5S)-3-(3-(3-bromo-2-methylphenoxy)propyl)-3,8-diazabicyclo[3.2.1]octane (2 g, crude) as a white solid.

MS (ESI) m/z: 340.8 [M+H]⁺.

### Step C. Procedure for Preparation of ethyl 2-((1R, 5S)-3-(3-(3-bromo-2-methylphenoxy)propyl)-3,8-diazabicyclo[3.2.1]octan-8-yl)acetate

A mixture of (1R, 5S)-3-(3-(3-bromo-2-methylphenoxy)propyl)-3,8-diazabicyclo[3.2.1]octane (1 g, 2.95 mmol, 1 equiv.), ethyl 2-bromoacetate (984.47 mg, 5.90 mmol, 651.97 μL, 2 equiv.) and DIEA (1.14 g, 8.84 mmol, 1.54 mL, 3 equiv.) in CH₃CN (30 mL) was degassed and purged with N₂ three times, and then the mixture was stirred at 60° C. for 16 hours under N₂ atmosphere. The reaction mixture was diluted with water 50 mL and extracted with EtOAc 50 mL×3. The combined organic layers were washed with brine 50 mL×3, dried over anhydrous sodium sulfate, filtered, and concentrated under reduced pressure to give a residue. The residue was purified by reverse-phase HPLC (with 0.1% HCOOH) to give compound ethyl 2-((1R, 5S)-3-(3-(3-bromo-2-methylphenoxy)propyl)-3,8-diazabicyclo[3.2.1]octan-8-yl)acetate (800 mg, 1.84 mmol, 62.5% yield, 98% purity) as a yellow oil.

MS (ESI) m/z: 426.9 [M+H]⁺.

### Step D. Procedure for Preparation of tert-butyl 6-(8-(benzo[d]thiazol-2-ylcarbamoyl)-3,4-dihydroisoquinolin-2(1H)-yl)-3-(3-(3-((1R,5S)-8-(2-ethoxy-2-oxoethyl)-3,8-diazabicyclo[3.2.1]octan-3-yl)propoxy)-2-methylphenyl)picolinate

Ethyl 2-((1R, 5S)-3-(3-(3-bromo-2-methylphenoxy)propyl)-3,8-diazabicyclo[3.2.1]octan-8-yl)acetate (150 mg, 352.64 μmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (216.01 mg, 352.64 μmol, 1 equiv.), KF (1.5 M, 705.29 μL, 3 equiv.) and Ad₂nBuP Pd G₃ (cataCXium® A Pd G₃) (25.68 mg, 35.26 μmol, 0.1 equiv.) were taken up into a microwave tube in 1,4-dioxane (3.5 mL). The sealed tube was heated at 100° C. for 60 minutes under microwave. The reaction mixture was diluted with water 20 mL and extracted with DCM 25 mL×3. The combined organic layers were washed with brine 15 mL×3, dried over anhydrous sodium sulfate, filtered, and concentrated under reduced pressure to give a residue. The residue was purified by prep-TLC (SiO₂, DCM:MeOH=10:1) to give compound tert-butyl 6-(8-(benzo[d]thiazol-2-ylcarbamoyl)-3,4-dihydroisoquinolin-2(1H)-yl)-3-(3-(3-((1R,5S)-8-(2-ethoxy-2-oxoethyl)-3,8-diazabicyclo[3.2.1]octan-3-yl)propoxy)-2-methylphenyl)picolinate (190 mg, 225.84 μmol, 64.04% yield, 98.78% purity) as a yellow oil.

MS (ESI) m/z: 831.7 [M+H]⁺.

### Step E. Procedure for Preparation of 2-((1R,5S)-3-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-3,8-diazabicyclo[3.2.1]octan-8-yl)acetic acid

A mixture of tert-butyl 6-(8-(benzo[d]thiazol-2-ylcarbamoyl)-3,4-dihydroisoquinolin-2(1H)-yl)-3-(3-(3-((1R,5S)-8-(2-ethoxy-2-oxoethyl)-3,8-diazabicyclo[3.2.1]octan-3-yl)propoxy)-2-methylphenyl)picolinate (190 mg, 228.63 μmol, 1 equiv.) and LiOH H₂O (28.78 mg, 685.89 μmol, 3 equiv.) in THE (1.5 mL) and H₂O (0.5 mL) was degassed and purged with N₂ three times, and then the mixture was stirred at 25° C. for 1 hour under N₂ atmosphere. The mixture was concentrated under reduced pressure to give compound 2-((1R,5S)-3-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-3,8-diazabicyclo[3.2.1]octan-8-yl)acetic acid (150 mg, 186.8 μmol, 81.7% yield) as a white solid.

MS (ESI) m/z: 803.5 [M+H]⁺.

### Step F. Procedure for Preparation of tert-butyl 6-(8-(benzo[d]thiazol-2-ylcarbamoyl)-3,4-dihydroisoquinolin-2(1H)-yl)-3-(3-(3-((1R,5S)-8-(2-((3-(2,6-dioxopiperidin-3-yl)-1-methyl-1H-indazol-6-yl)amino)-2-oxoethyl)-3,8-diazabicyclo[3.2.1]octan-3-yl)propoxy)-2-methylphenyl)picolinate

To a solution of 2-((1R,5S)-3-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-3,8-diazabicyclo[3.2.1]octan-8-yl)acetic acid (100 mg, 124.54 μmol, 1 equiv.) in DMF (2 mL) was added DIEA (48.29 mg, 373.61 μmol, 65.07 uL, 3 equiv.) and HATU (56.82 mg, 149.44 μmol, 1.2 equiv.), 3-(6-amino-1-methyl-1H-indazol-3-yl)piperidine-2,6-dione (35.38 mg, 136.99 μmol, 1.1 equiv.). The mixture was stirred at 25° C. for 1 hour. The reaction was diluted with water (30 mL) and extracted with DCM (20 mL×2). The combined organic layers were washed with brine (30 mL×3), dried over Na₂SO₄, filtered, and concentrated under reduced pressure to give a residue. The residue was purified by prep-TLC (SiO₂, DCM:MeOH=10:1, R_{f}=0.5) to give compound tert-butyl 6-(8-(benzo[d]thiazol-2-ylcarbamoyl)-3,4-dihydroisoquinolin-2(1H)-yl)-3-(3-(3-((1R,5S)-8-(2-((3-(2,6-dioxopiperidin-3-yl)-1-methyl-1H-indazol-6-yl)amino)-2-oxoethyl)-3,8-diazabicyclo[3.2.1]octan-3-yl)propoxy)-2-methylphenyl)picolinate (120 mg, 115.0 μmol, 92.3% yield) as a yellow solid.

MS (ESI) m/z: 1043.1 [M+H]⁺.

### Step G. Procedure for Preparation of 6-(8-(benzo[d]thiazol-2-ylcarbamoyl)-3,4-dihydroisoquinolin-2(1H)-yl)-3-(3-(3-((1R,5S)-8-(2-((3-(2,6-dioxopiperidin-3-yl)-1-methyl-1H-indazol-6-yl)amino)-2-oxoethyl)-3,8-diazabicyclo[3.2.1]octan-3-yl)propoxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(3-(3-((1R,5S)-8-(2-((3-(2,6-dioxopiperidin-3-yl)-1-methyl-1H-indazol-6-yl)amino)-2-oxoethyl)-3,8-diazabicyclo[3.2.1]octan-3-yl)propoxy)-2-methylphenyl)picolinate (100 mg, 95.86 μmol, 1 equiv.) in TFA (0.5 mL) and DCM (0.5 mL) was degassed and purged with N₂ three times, and then the mixture was stirred at 25° C. for 1 hour under N₂ atmosphere. The reaction mixture was filtered and concentrated under reduced pressure to give a residue. The residue was purified by prep-HPLC to give compound 6-(8-(benzo[d]thiazol-2-ylcarbamoyl)-3,4-dihydroisoquinolin-2(1H)-yl)-3-(3-(3-((1R,5S)-8-(2-((3-(2,6-dioxopiperidin-3-yl)-1-methyl-1H-indazol-6-yl)amino)-2-oxoethyl)-3,8-diazabicyclo[3.2.1]octan-3-yl)propoxy)-2-methylphenyl)picolinic acid (28.6 mg, 28.9 μmol, 30.1% yield, 99.5% purity) as a white solid.

MS (ESI) m/z: 494.3 [M12+H]⁺.

¹H NMR (400 MHz, DMSO-d₆) δ=10.86 (s, 1H), 9.93 (s, 1H), 8.07-8.00 (m, 2H), 7.78 (d, J=8.0 Hz, 1H), 7.67-7.59 (m, 2H), 7.49-7.42 (m, 3H), 7.39-7.32 (m, 2H), 7.20 (dd, J=8.8, 2.0 Hz, 1H), 7.14-7.07 (m, 1H), 6.96 (d, J=8.8 Hz, 1H), 6.89 (d, J=8.0 Hz, 1H), 6.64 (d, J=7.6 Hz, 1H), 4.98 (s, 2H), 4.33 (q, J=4.8 Hz, 1H), 4.02 (t, J=6.0 Hz, 2H), 3.94-3.91 (m, 4H), 3.20 (s, 4H), 3.12 (s, 3H), 3.03 (t, J=5.6 Hz, 2H), 2.68-2.61 (m, 4H), 2.42-2.23 (m, 4H), 2.21-2.14 (m, 1H), 1.90 (s, 3H), 1.87 (d, J=6.4 Hz, 1H), 1.85-1.79 (m, 2H), 1.75-1.68 (m, 2H).

### Example 22. Preparation of Compound 131a

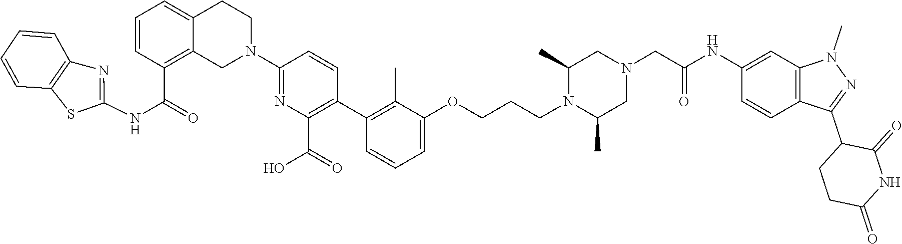 [SMILES: Cc1c(OCCCN2[C@H](C)CN(CC(=O)Nc3ccc4c(C5CCC(=O)NC5=O)nn(C)c4c3)C[C@@H]2C)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[(2R,6S)-4-[2-[[3-(2,6-dioxo-3-piperidyl)-1-methyl-indazol-6-yl]amino]-2-oxo-ethyl]-2,6-dimethyl-piperazin-1-yl]propoxy]-2-methyl-phenyl]pyridine-2-carboxylic acid

### Step A. Procedure for Preparation of tert-butyl (3R,5S)-4-(3-iodopropyl)-3,5-dimethyl-piperazine-1-carboxylate

To a solution of tert-butyl (3R,5S)-3,5-dimethylpiperazine-1-carboxylate (2.0 g, 9.33 mmol, 1 equiv.) in CH₃CN (5 mL) was added 1,3-diiodopropane (2.76 g, 9.33 mmol, 1.08 mL, 1 equiv.) and K₂CO₃ (1.29 g, 9.33 mmol, 1 equiv.). The mixture was stirred at 60° C. for 12 hours. The reaction mixture was concentrated under reduced pressure to remove solvent to give a residue. The residue was purified by column chromatography (SiO₂, DCM:MeOH=10:1) to give the compound tert-butyl (3R,5S)-4-(3-iodopropyl)-3,5-dimethyl-piperazine-1-carboxylate (1.0 g, 2.62 mmol, 28.0% yield) as a yellow oil.

¹H NMR (400 MHz, CDCl₃) δ=5.23-5.07 (m, 1H), 3.96-3.58 (m, 2H), 3.39 (s, 1H), 3.05 (t, J=6.8 Hz, 1H), 2.81-2.71 (m, 1H), 2.64-2.28 (m, 4H), 1.88-1.78 (m, 1H), 1.52 (d, J=14.8 Hz, 1H), 1.39 (s, 9H), 1.02 (d, J=5.2 Hz, 6H).

### Step B. Procedure for Preparation of tert-butyl (3R,5S)-4-[3-(3-bromo-2-methyl-phenoxy)propyl]-3,5-dimethyl-piperazine-1-carboxylate

To a solution of tert-butyl (3R,5S)-4-(3-iodopropyl)-3,5-dimethyl-piperazine-1-carboxylate (1 g, 2.62 mmol, 1 equiv.) in CH₃CN (15 mL) was added K₂CO₃ (1.08 g, 7.85 mmol, 3 equiv.) and 3-bromo-2-methyl-phenol (489.26 mg, 2.62 mmol, 1 equiv.). The mixture was stirred at 60° C. for 12 hours. The reaction mixture was filtered and concentrated under reduced pressure to give a residue. The residue was purified by column chromatography (SiO₂, DCM:MeOH=1/0 to 10/1) to give the compound tert-butyl (3R,5S)-4-[3-(3-bromo-2-methyl-phenoxy)propyl]-3,5-dimethyl-piperazine-1-carboxylate (540 mg, 1.22 mmol, 46.7% yield) as a yellow oil.

MS (ESI) m/z: 443.3 [M+H]⁺

### Step C. Procedure for Preparation of (2R,6S)-1-[3-(3-bromo-2-methyl-phenoxy)propyl]-2,6-dimethyl-piperazine

To a solution of tert-butyl (3R,5S)-4-[3-(3-bromo-2-methyl-phenoxy)propyl]-3,5-dimethyl-piperazine-1-carboxylate (500 mg, 1.13 mmol, 1 equiv.) in EtOAc (5 mL) was added HCl/EtOAc (3 M, 1.13 mL, 3 equiv.). The mixture was stirred at 25° C. for 12 hours. The reaction mixture was concentrated under reduced pressure to remove EtOAc to give the crude product (2R,6S)-1-[3-(3-bromo-2-methyl-phenoxy)propyl]-2,6-dimethyl-piperazine (420 mg, 1.11 mmol, 98.1% yield) as a white solid.

MS (ESI) m/z: 342.8 [M+H]⁺

### Step D. Procedure for Preparation of ethyl 2-[(3R,5S)-4-[3-(3-bromo-2-methyl-phenoxy)propyl]-3,5-dimethyl-piperazin-1-yl]acetate

To a solution of (2R,6S)-1-[3-(3-bromo-2-methyl-phenoxy)propyl]-2,6-dimethyl-piperazine (420 mg, 1.23 mmol, 1 equiv.) in CH₃CN (10 mL) was added K₂CO₃ (510.25 mg, 3.69 mmol, 3 equiv.) and ethyl 2-bromoacetate (205.52 mg, 1.23 mmol, 136.10 μL, 1 equiv.). The mixture was stirred at 60° C. for 12 hours. The reaction mixture was filtered, washed by EtOAc (10 mL), and concentrated under reduced pressure to give a residue. The residue was purified by column chromatography (SiO₂, DCM:MeOH=1/0 to 10/1) to give the compound ethyl 2-[(3R,5S)-4-[3-(3-bromo-2-methyl-phenoxy)propyl]-3,5-dimethyl-piperazin-1-yl]acetate (350 mg, 818.9 μmol, 66.5% yield) as a pink solid.

MS (ESI) m/z: 429.3 [M+H]⁺

### Step E. Procedure for Preparation of tert-butyl 6-[8-(1,3-benzothiazol-2-ylcarbamoyl)-3,4-dihydro-1H-isoquinolin-2-yl]-3-[3-[3-[(2R,6S)-4-(2-ethoxy-2-oxo-ethyl)-2,6-dimethyl-piperazin-1-yl]propoxy]-2-methyl-phenyl]pyridine-2-carboxylate

To a solution of ethyl 2-[(3R,5S)-4-[3-(3-bromo-2-methyl-phenoxy)propyl]-3,5-dimethyl-piperazin-1-yl]acetate (250 mg, 584.97 μmol, 1 equiv.) taken up into a microwave tube in dioxane (5 mL) and H₂O (1 mL) was added tert-butyl 6-[8-(3H-indol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (347.77 mg, 584.97 μmol, 1 equiv.), Ad₂nBuP Pd G₃(cataCXium® A Pd G₃) (42.60 mg, 58.50 μmol, 0.1 equiv.), and K₂CO₃ (242.54 mg, 1.75 mmol, 3 equiv.). The reaction mixture was degassed and purged with N₂ three times and was then stirred at 100° C. for 1 hour under N₂ atmosphere. The reaction mixture was concentrated under reduced pressure to give the crude product. The crude product was purified by reverse-phase HPLC to give the compound tert-butyl 6-[8-(1,3-benzothiazol-2-ylcarbamoyl)-3,4-dihydro-1H-isoquinolin-2-yl]-3-[3-[3-[(2R,6S)-4-(2-ethoxy-2-oxo-ethyl)-2,6-dimethyl-piperazin-1-yl]propoxy]-2-methyl-phenyl]pyridine-2-carboxylate (100 mg, 120.04 μmol, 20.52% yield) as a yellow oil.

¹H NMR (400 MHz, CDCl₃) δ=8.23 (s, 2H), 7.78 (d, J=7.6 Hz, 1H), 7.62-7.56 (m, 2H), 7.41-7.24 (m, 6H), 7.21-7.16 (m, 2H), 7.04 (t, J=8.0 Hz, 1H), 6.83-6.78 (m, 1H), 6.73-6.64 (m, 2H), 5.04-4.89 (m, 2H), 4.12 (q, J=7.2 Hz, 2H), 4.06-3.90 (m, 4H), 3.40-3.20 (m, 4H), 3.19-3.16 (m, 2H), 3.03-2.96 (m, 2H), 2.87 (d, J=11.2 Hz, 2H), 2.69-2.60 (m, 2H), 2.08-1.97 (m, 2H), 1.92-1.88 (m, 3H), 1.30 (s, 6H), 1.23-1.18 (m, 3H), 1.09 (s, 9H).

### Step F. Procedure for Preparation of 2-[(3R,5S)-4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-3,5-dimethyl-piperazin-1-yl]acetic acid

To a solution of tert-butyl 6-[8-(1,3-benzothiazol-2-ylcarbamoyl)-3,4-dihydro-1H-isoquinolin-2-yl]-3-[3-[3-[(2R,6S)-4-(2-ethoxy-2-oxo-ethyl)-2,6-dimethyl-piperazin-1-yl]propoxy]-2-methyl-phenyl]pyridine-2-carboxylate (100 mg, 120.04 μmol, 1 equiv.) in THE (3 mL) and H₂O (1 mL) was added LiOH·H₂O (50.4 mg, 12.00 μmol, 10 equiv.). The mixture was stirred at 25° C. for 12 hours. The reaction mixture was concentrated under reduced pressure to remove solvent. The resulting residue was diluted with H₂O (10 mL) and adjusted to pH=3-4. The mixture was then extracted with EtOAc 10 mL (10 mL×3), and the combined organic layers were washed with brine (10 mL), filtered and concentrated under reduced pressure to give the crude product 2-[(3R,5S)-4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-3,5-dimethyl-piperazin-1-yl]acetic acid (84 mg, 104.3 μmol, 86.9% yield, 100% purity) as a yellow solid.

MS (ESI) m/z: 805.2 [M+H]⁺

### Step G. Procedure for Preparation of tert-butyl 6-[8-(1,3-benzothiazol-2-ylcarbamoyl)-3,4-dihydro-1H-isoquinolin-2-yl]-3-[3-[3-[(2R,6S)-4-[2-[[3-(2,6-dioxo-3-piperidyl)-1-methyl-indazol-6-yl]amino]-2-oxo-ethyl]-2,6-dimethyl-piperazin-1-yl]propoxy]-2-methyl-phenyl]pyridine-2-carboxylate

To a solution of 2-[(3R,5S)-4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-3,5-dimethyl-piperazin-1-yl]acetic acid (84 mg, 104.35 μmol, 1 equiv.) in DMF (1 mL) was added 3-(6-amino-1-methyl-indazol-3-yl)piperidine-2,6-dione (26.95 mg, 104.35 μmol, 1 equiv.), HATU (39.68 mg, 104.35 μmol, 1 equiv.) and DIEA (13.49 mg, 104.35 μmol, 18.18 μL, 1 equiv.). The mixture was stirred at 25° C. for 2 hours. The reaction mixture was diluted with water (10 mL) and extracted with EtOAc (10 mL). The combined extracts were filtered and concentrated under reduced pressure to give the crude product tert-butyl 6-[8-(1,3-benzothiazol-2-ylcarbamoyl)-3,4-dihydro-1H-isoquinolin-2-yl]-3-[3-[3-[(2R,6S)-4-[2-[[3-(2,6-dioxo-3-piperidyl)-1-methyl-indazol-6-yl]amino]-2-oxo-ethyl]-2,6-dimethyl-piperazin-1-yl]propoxy]-2-methyl-phenyl]pyridine-2-carboxylate (80 mg, 67.3 μmol, 64.5% yield, 88% purity) as a yellow solid.

MS (ESI) m/z: 523.2 [M12+H]⁺

### Step H. Procedure for Preparation of 6-[8-(1,3-benzothiazol-2-ylcarbamoyl)-3,4-dihydro-1H-isoquinolin-2-yl]-3-[3-[3-[(2R,6S)-4-[2-[[3-(2,6-dioxo-3-piperidyl)-1-methyl-indazol-6-yl]amino]-2-oxo-ethyl]-2,6-dimethyl-piperazin-1-yl]propoxy]-2-methyl-phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[3-[(2R,6S)-4-[2-[[3-(2,6-dioxo-3-piperidyl)-1-methyl-indazol-6-yl]amino]-2-oxo-ethyl]-2,6-dimethyl-piperazin-1-yl]propoxy]-2-methyl-phenyl]pyridine-2-carboxylate (70 mg, 66.97 μmol, 1 equiv.) in TFA (1 mL) and DCM (1 mL) was stirred at 25° C. for 12 hours. The reaction mixture was concentrated under reduced pressure to give the crude product. The crude product was purified by reverse-phase HPLC to give the compound 6-[8-(1,3-benzothiazol-2-ylcarbamoyl)-3,4-dihydro-1H-isoquinolin-2-yl]-3-[3-[3-[(2R,6S)-4-[2-[[3-(2,6-dioxo-3-piperidyl)-1-methyl-indazol-6-yl]amino]-2-oxo-ethyl]-2,6-dimethyl-piperazin-1-yl]propoxy]-2-methyl-phenyl]pyridine-2-carboxylic acid (41.2 mg, 39.6 μmol, 59.2% yield, 94.9% purity) as a white solid.

MS (ESI) m/z: 989.7 [M+H]⁺.

¹H NMR (400 MHz, DMSO-d₆) δ=10.90 (s, 1H), 9.90-9.85 (m, 1H), 8.16-8.14 (m, 1H), 8.08-8.01 (m, 2H), 7.82-7.77 (m, 1H), 7.63 (d, J=8.8 Hz, 2H), 7.50-7.42 (m, 3H), 7.41-7.33 (m, 2H), 7.22-7.17 (m, 1H), 7.14-7.07 (m, 1H), 7.00-6.94 (m, 1H), 6.92-6.84 (m, 1H), 6.64 (d, J=7.6 Hz, 1H), 5.02-4.95 (m, 2H), 4.36-4.30 (m, 1H), 4.03-3.96 (m, 2H), 3.95-3.89 (m, 5H), 3.12 (s, 2H), 3.03 (t, J=5.6 Hz, 2H), 2.96-2.88 (m, 2H), 2.78 (d, J=9.2 Hz, 4H), 2.64 (dt, J=5.6, 10.4 Hz, 2H), 2.41-2.30 (m, 1H), 2.22-2.13 (m, 1H), 2.07-1.97 (m, 2H), 1.94-1.90 (m, 3H), 1.90-1.80 (m, 2H), 1.07-0.99 (m, 6H).

### Example 23. Preparation of Compound 135

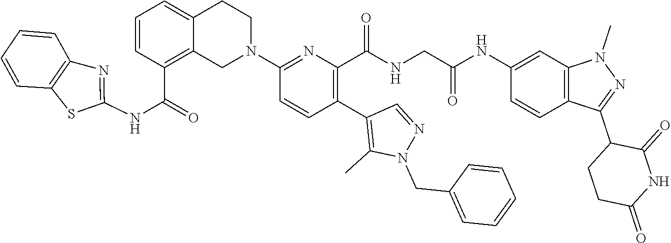 [SMILES: Cc1c(-c2ccc(N3CCc4cccc(C(=O)Nc5nc6ccccc6s5)c4C3)nc2C(=O)NCC(=O)Nc2ccc3c(C4CCC(=O)NC4=O)nn(C)c3c2)cnn1Cc1ccccc1]

### N-(1,3-benzothiazol-2-yl)-2-[5-(1-benzyl-5-methyl-pyrazol-4-yl)-6-[[2-[[3-(2,6-dioxo-3-piperidyl)-1-methyl-indazol-6-yl]amino]-2-oxo-ethyl]carbamoyl]-2-pyridyl]-3,4-dihydro-1H-isoquinoline-8-carboxamide

### Step A. Procedure for Preparation of 1-benzyl-4-iodo-pyrazole

To a solution of bromomethylbenzene (13.6 g, 79.6 mmol, 9.45 mL, 1.05 equiv.) and 4-iodo-1H-pyrazole (14.7 g, 75.8 mmol, 1 equiv.) in acetone (200 mL) was added K₂CO₃ (26.2 g, 189 mmol, 2.5 equiv.). The mixture was stirred at 70° C. for 4 h. After completion, the reaction was filtered and concentrated under reduced pressure to give the crude product. The crude product was purified by flash silica gel chromatography (eluent of 0˜10% ethyl acetate/petroleum ether). 1-Benzyl-4-iodo-pyrazole (20.4 g, 68.2 mmol, 90.0% yield, 95% purity) was obtained as an off-white solid.

¹H NMR (400 MHz, CD₃Cl) δ=7.46 (s, 1H), 7.31 (s, 1H), 7.22-7.28 (m, 3H), 7.14 (dd, J=8.0, 1.75 Hz, 2H), 5.21 (s, 2H)

### Step B. Procedure for Preparation of 1-benzyl-4-iodo-5-methyl-pyrazole

To a solution of 1-benzyl-4-iodo-pyrazole (10.0 g, 35.2 mmol, 1 equiv.) in THE (100 mL) was added LDA (2 M, 21.1 mL, 1.2 equiv.) at −70° C., and then the mixture was stirred at −70° C. for 30 min. Mel (5.50 g, 38.7 mmol, 2.41 mL, 1.1 equiv.) was added dropwise at −70° C. Then the mixture was stirred at 25° C. for 5 h. After completion, the reaction was quenched with a saturated solution of NH₄Cl (50 ml) and extracted with ethyl acetate (70 ml×3). The combined organic layers were dried over Na₂SO₄, filtered, and concentrated under reduced pressure to give 1-benzyl-4-iodo-5-methyl-pyrazole (9.6 g, crude) as a white solid.

MS (ESI) m/z: 298.7 [M+H]⁺.

### Step C. Procedure for Preparation of 1-benzyl-5-methyl-4-(4,4,5,5-tetramethyl-1,3,2-dioxaborolan-2-yl)pyrazole

To a solution of 1-benzyl-4-iodo-5-methyl-pyrazole (8.50 g, 28.5 mmol, 1 equiv.) and 4,4,5,5-tetramethyl-1,3,2-dioxaborolane (14.6 g, 114 mmol, 16.6 mL, 4 equiv.) in CH₃CN (100 mL) was added TEA (11.5 g, 114 mmol, 15.9 mL, 4 equiv.) and cyclopentyl(diphenyl)phosphane; dichloromethane; dichloropalladium; iron (2.33 g, 2.85 mmol, 0.1 equiv.). The mixture was stirred at 80° C. for 4 h under N₂ atmosphere. The reaction mixture was partitioned between water (100 mL) and ethyl acetate (200 mL). The organic phase was separated, washed with ethyl acetate (200 mL×2), dried over Na₂SO₄, filtered, and concentrated under reduced pressure to give a residue. The residue was purified by flash silica gel chromatography (eluent of 0˜20% ethyl acetate/petroleum ether) to give 1-benzyl-5-methyl-4-(4,4,5,5-tetramethyl-1,3,2-dioxaborolan-2-yl)pyrazole (7.00 g, 21.1 mmol, 74.1% yield, 90% purity) as a white solid.

¹H NMR (400 MHz, CDCl₃) δ=7.67 (s, 1H), 7.13-7.27 (m, 3H), 7.02 (d, J=7.2 Hz, 2H), 5.22 (s, 2H), 2.30 (s, 3H), 1.15-1.23 (m, 12H)

### Step D. Procedure for Preparation of tert-butyl 6-[8-(1,3-benzothiazol-2-ylcarbamoyl)-3,4-dihydro-1H-isoquinolin-2-yl]-3-(1-benzyl-5-methyl-pyrazol-4-yl)pyridine-2-carboxylate

To a solution of 1-benzyl-5-methyl-4-(4,4,5,5-tetramethyl-1,3,2-dioxaborolan-2-yl)pyrazole (571 mg, 1.92 mmol, 1.3 equiv.) and tert-butyl 6-[8-(1,3-benzothiazol-2-ylcarbamoyl)-3,4-dihydro-1H-isoquinolin-2-yl]-3-bromo-pyridine-2-carboxylate (833 mg, 1.47 mmol, 1 equiv.) in dioxane (10 mL) and H₂O (3 mL) was added [2-(2-aminophenyl)phenyl]palladium(1+); bis(1-adamantyl)-butyl-phosphane; methanesulfonate (215 mg, 295 μmol, 0.2 equiv.) and K₂CO₃ (611 mg, 4.42 mmol, 3 equiv.). The mixture was stirred at 100° C. for 1 h under N₂ atmosphere. The reaction mixture was partitioned between water (30 mL) and ethyl acetate (30 mL). The organic phase was separated, washed with ethyl acetate (30 mL×2), dried over Na₂SO₄, filtered, and concentrated under reduced pressure to give a residue. The residue was purified by flash silica gel chromatography (eluent of 0˜10% methanol/dichloromethane ether). tert-Butyl 6-[8-(1,3-benzothiazol-2-ylcarbamoyl)-3,4-dihydro-1H-isoquinolin-2-yl]-3-(1-benzyl-5-methyl-pyrazol-4-yl)pyridine-2-carboxylate (0.9 g, 1.23 mmol, 83.7% yield, 90% purity) was obtained as a yellow solid which was used in the next step directly.

MS (ESI) m/z: 657.4 [M+H]⁺.

### Step E. Procedure for Preparation of 6-[8-(1,3-benzothiazol-2-ylcarbamoyl)-3,4-dihydro-1H-isoquinolin-2-yl]-3-(1-benzyl-5-methyl-pyrazol-4-yl)pyridine-2-carboxylic acid

To a solution of tert-butyl 6-[8-(1,3-benzothiazol-2-ylcarbamoyl)-3,4-dihydro-1H-isoquinolin-2-yl]-3-(1-benzyl-5-methyl-pyrazol-4-yl)pyridine-2-carboxylate (0.9 g, 1.37 mmol, 1 equiv.) in DCM (10 mL) was added TFA (7.70 g, 67.5 mmol, 5 mL, 49.3 equiv.). The mixture was stirred at 25° C. for 16 h. The reaction mixture was concentrated under reduced pressure to give 6-[8-(1,3-benzothiazol-2-ylcarbamoyl)-3,4-dihydro-1H-isoquinolin-2-yl]-3-(1-benzyl-5-methyl-pyrazol-4-yl)pyridine-2-carboxylic acid (867 mg, crude) as a yellow oil which was used in next step directly.

### Step F. Procedure for Preparation of methyl 2-[[6-[8-(1,3-benzothiazol-2-ylcarbamoyl)-3,4-dihydro-1H-isoquinolin-2-yl]-3-(1-benzyl-5-methyl-pyrazol-4-yl)pyridine-2-carbonyl]amino]acetate

To a solution of 6-[8-(1,3-benzothiazol-2-ylcarbamoyl)-3,4-dihydro-1H-isoquinolin-2-yl]-3-(1-benzyl-5-methyl-pyrazol-4-yl)pyridine-2-carboxylic acid (260 mg, 433 μmol, 1 equiv.) in DMF (5 mL) was added HATU (197 mg, 519 μmol, 1.2 equiv.) and DIEA (168 mg, 1.30 mmol, 226 μL, 3 equiv.). The mixture was stirred at 25° C. for 30 min. Methyl 2-aminoacetate (65.2 mg, 519 μmol, 1.2 equiv., HCl) was added into the mixture and stirred at 25° C. for 1 h. To the reaction mixture was added water (50 mL), then the mixture was filtered to give a crude product. Methyl 2-[[6-[8-(1,3-benzothiazol-2-ylcarbamoyl)-3,4-dihydro-1H-isoquinolin-2-yl]-3-(1-benzyl-5-methyl-pyrazol-4-yl)pyridine-2-carbonyl]amino]acetate (0.2 g, crude) was obtained as a yellow solid which was used in the next step directly.

MS (ESI) m/z: 672.5 [M+H]⁺.

### Step G. Procedure for Preparation of 2-[[6-[8-(1,3-benzothiazol-2-ylcarbamoyl)-3,4-dihydro-1H-isoquinolin-2-yl]-3-(1-benzyl-5-methyl-pyrazol-4-yl)pyridine-2-carbonyl]amino]acetic acid

To a solution of methyl 2-[[6-[8-(1,3-benzothiazol-2-ylcarbamoyl)-3,4-dihydro-1H-isoquinolin-2-yl]-3-(1-benzyl-5-methyl-pyrazol-4-yl)pyridine-2-carbonyl]amino]acetate (180 mg, 268 μmol, 1 equiv.) in THF (3 mL) was added LiOH·H₂O (0.5 M, 1 mL, 1.87 equiv.). The mixture was stirred at 25° C. for 1 h. The reaction mixture was acidified with aqueous HCl (1 M) till pH=3, and then the mixture was filtered to give 2-[[6-[8-(1,3-benzothiazol-2-ylcarbamoyl)-3,4-dihydro-1H-isoquinolin-2-yl]-3-(1-benzyl-5-methyl-pyrazol-4-yl)pyridine-2-carbonyl]amino]acetic acid (100 mg, crude) as a white solid which was used in the next step directly.

MS (ESI) m/z: 658.1 [M+H]⁺.

### Step H. Procedure for Preparation of N-(1,3-benzothiazol-2-yl)-2-[5-(1-benzyl-5-methyl-pyrazol-4-yl)-6-[[2-[[3-(2,6-dioxo-3-piperidyl)-1-methyl-indazol-6-yl]amino]-2-oxo-ethyl]carbamoyl]-2-pyridyl]-3,4-dihydro-1H-isoquinoline-8-carboxamide

To a solution of 3-(6-amino-1-methyl-indazol-3-yl)piperidine-2,6-dione (47.1 mg, 182 μmol, 1.2 equiv.) in DMF (3 mL) was added HATU (63.6 mg, 167.24 μmol, 1.1 equiv.) and DIEA (58.9 mg, 456 μmol, 79.4 μL, 3 equiv.), and then the mixture was stirred at 25° C. for 30 min. 2-[[6-[8-(1,3-benzothiazol-2-ylcarbamoyl)-3,4-dihydro-1H-isoquinolin-2-yl]-3-(1-benzyl-5-methyl-pyrazol-4-yl)pyridine-2-carbonyl]amino]acetic acid (100 mg, 152 μmol, 1 equiv.) was added into the mixture and stirred at 25° C. for 30 min. The reaction mixture was filtered. The filtrate was purified by prep-HPLC. N-(1,3-benzothiazol-2-yl)-2-[5-(1-benzyl-5-methyl-pyrazol-4-yl)-6-[[2-[[3-(2,6-dioxo-3-piperidyl)-1-methyl-indazol-6-yl]amino]-2-oxo-ethyl]carbamoyl]-2-pyridyl]-3,4-dihydro-1H-isoquinoline-8-carboxamide (10.6 mg, 10.9 μmol, 7.20% yield, 93% purity) was obtained as a yellow solid.

¹H NMR (400 MHz, DMSO-d₆) 6 ppm=10.88 (s, 1H), 10.16 (s, 1H), 8.71 (t, J=5.6 Hz, 1H), 7.95 (s, 1H), 7.78 (d, J=7.2 Hz, 1H), 7.69 (m, 1H), 7.57-7.64 (m, 2H), 7.51 (d, J=8.8 Hz, 1H), 7.24-7.41 (m, 7H), 7.17-7.24 (m, 2H), 7.02-7.13 (m, 4H), 5.27 (s, 2H), 5.05 (s, 2H), 4.32 (m, 1H), 4.07 (m, 2H), 3.98 (s, 2H), 3.87 (s, 3H), 2.96-3.07 (m, 3H), 2.62-2.67 (m, 1H), 2.27-2.35 (m, 1H), 2.16 (s, 1H), 2.03 (s, 3H)

### Example 24. Preparation of Compound 137

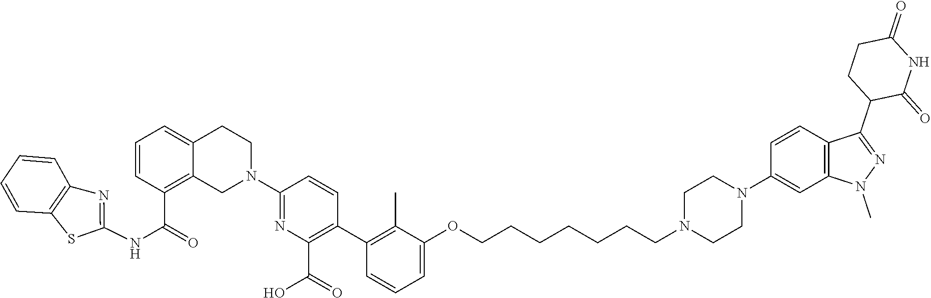 [SMILES: Cc1c(OCCCCCCCN2CCN(c3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-((7-(4-(3-(2,6-dioxopiperidin-3-yl)-1-methyl-1H-indazol-6-yl)piperazin-1-yl)heptyl)oxy)-2-methylphenyl)picolinic acid

### Step A. Procedure for Preparation of tert-butyl 4-[3-(2,6-dibenzyloxy-3-pyridyl)-1-methyl-indazol-6-yl]piperazine-1-carboxylate

To a solution of 6-bromo-3-(2,6-dibenzyloxy-3-pyridyl)-1-methyl-indazole (5 g, 9.99 mmol, 1.00 equiv.) in dioxane (60 mL) was added RuPhos (932.55 mg, 2.00 mmol, 0.20 equiv.), Cs₂CO₃ (9.77 g, 29.98 mmol, 3.00 equiv.), tert-butyl piperazine-1-carboxylate (5.58 g, 29.98 mmol, 3.00 equiv.) and Pd(dba)₂ (574.56 mg, 999.23 μmol, 0.10 equiv.). The mixture was stirred at 100° C. for 3 hours. The reaction mixture was concentrated under reduced pressure to remove solvent and then diluted with H₂O (100 mL) and extracted with ethyl acetate (100 mL×3). The combined organic layers were washed with H₂O (100 mL×3), dried over Na₂SO₄, filtered, and concentrated under reduced pressure to give a residue. The residue was purified by column chromatography (SiO₂, petroleum ether/ethyl acetate=100/1 to 3/1) to give tert-butyl 4-[3-(2,6-dibenzyloxy-3-pyridyl)-1-methyl-indazol-6-yl]piperazine-1-carboxylate (5.6 g, 9.25 mmol, 92.52% yield) as a yellow oil.

MS (ESI) m/z: 606.5 [M+H]⁺.

### Step B. Procedure for Preparation of tert-butyl 4-(3-(2,6-dioxopiperidin-3-yl)-1-methyl-1H-indazol-6-yl)piperazine-1-carboxylate

To a solution of tert-butyl 4-[3-(2,6-dibenzyloxy-3-pyridyl)-1-methyl-indazol-6-yl]piperazine-1-carboxylate (5.6 g, 9.25 mmol, 1.00 equiv.) in THE (40 mL) and EtOH (30 mL) was added Pd(OH)₂ (2.86 g, 4.07 mmol, 20% purity, 0.30 equiv.) and Pd/C (2.85 g, 2.72 mmol, 10% purity, 0.20 equiv.), AcOH (2.45 g, 40.73 mmol, 2.33 mL, 3.00 equiv.) under N₂ atmosphere. The suspension was degassed and purged with H₂ three times. The mixture was stirred under H₂ (50 Psi) at 50° C. for 12 hours. The reaction mixture was filtered and concentrated under reduced pressure to give tert-butyl 4-(3-(2,6-dioxopiperidin-3-yl)-1-methyl-1H-indazol-6-yl)piperazine-1-carboxylate (2.6 g, 6.08 mmol) as a white solid.

MS (ESI) m/z: 428.5 [M+H]⁺.

### Step C. Procedure for Preparation of 3-(1-methyl-6-piperazin-1-yl-indazol-3-yl)piperidine-2,6-dione

To a solution of tert-butyl 4-(3-(2,6-dioxopiperidin-3-yl)-1-methyl-1H-indazol-6-yl)piperazine-1-carboxylate (2.4 g, 5.61 mmol, 1.00 equiv.) in DCM (25 mL) was added HCl/dioxane (4 M, 1.40 mL, 1.00 equiv.). The mixture was stirred at 25° C. for 1 hour. The reaction mixture was concentrated under reduced pressure to give 3-(1-methyl-6-piperazin-1-yl-indazol-3-yl)piperidine-2,6-dione (1 g, 2.75 mmol, 48.9% yield) as a white solid.

¹H NMR (400 MHz, DMSO) δ=10.87 (s, 1H), 9.20 (s, 2H), 7.57 (d, J=8.8 Hz, 1H), 7.00-6.90 (m, 2H), 4.32-4.24 (m, 1H), 3.94-3.89 (m, 3H), 3.50-3.42 (m, 4H), 3.26 (s, 4H), 2.68-2.59 (m, 2H), 2.32-2.13 (m, 2H)

### Step D. Procedure for Preparation of tert-butyl 6-(8-(benzo[d]thiazol-2-ylcarbamoyl)-3,4-dihydroisoquinolin-2(1H)-yl)-3-(3-((7-(4-(3-(2,6-dioxopiperidin-3-yl)-1-methyl-1H-indazol-6-yl)piperazin-1-yl)heptyl)oxy)-2-methylphenyl)picolinate

To a solution of tert-butyl 6-(8-(benzo[d]thiazol-2-ylcarbamoyl)-3,4-dihydroisoquinolin-2(1H)-yl)-3-(2-methyl-3-((7-oxoheptyl)oxy)phenyl)picolinate (100 mg, 141.87 μmol, 1.00 equiv.) in DCM (1.5 mL) was added NMM (14.35 mg, 141.87 μmol, 15.60 μL, 1.00 equiv.) and 3-(1-methyl-6-piperazin-1-yl-indazol-3-yl)piperidine-2,6-dione (56.78 mg, 156.06 μmol, 1.10 equiv., HCl) at 25° C. for 4 hours. NaBH₃CN (26.75 mg, 425.61 μmol, 3.00 equiv.) was added to the mixture and stirred at 25° C. for 1 hour. The reaction was diluted with water (20 mL) and extracted with DCM 30 mL (30 mL×3). The combined organic layers were washed with brine 20 mL (20 mL×1), dried over Na₂SO₄, filtered and concentrated under reduced pressure to give tert-butyl 6-(8-(benzo[d]thiazol-2-ylcarbamoyl)-3,4-dihydroisoquinolin-2(1H)-yl)-3-(3-((7-(4-(3-(2,6-dioxopiperidin-3-yl)-1-methyl-1H-indazol-6-yl)piperazin-1-yl)heptyl)oxy)-2-methylphenyl)picolinate (100 mg, 95.6 μmol, 67.4% yield, 97.2% purity) was obtained as a yellow oil.

MS (ESI) m/z: 960.7 [M+H]⁺.

### Step E. Procedure for Preparation of 6-(8-(benzo[d]thiazol-2-ylcarbamoyl)-3,4-dihydroisoquinolin-2(1H)-yl)-3-(3-((7-(4-(3-(2,6-dioxopiperidin-3-yl)-1-methyl-1H-indazol-6-yl)piperazin-1-yl)heptyl)oxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-((7-(4-(3-(2,6-dioxopiperidin-3-yl)-1-methyl-1H-indazol-6-yl)piperazin-1-yl)heptyl)oxy)-2-methylphenyl)picolinate (100 mg, 98.40 μmol, 1.00 equiv.) in DCM (1 mL) was added TFA (1.54 g, 13.51 mmol, 1 mL, 137.26 equiv.). The mixture was stirred at 40° C. for 1 hour. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-((7-(4-(3-(2,6-dioxopiperidin-3-yl)-1-methyl-1H-indazol-6-yl)piperazin-1-yl)heptyl)oxy)-2-methylphenyl)picolinic acid (35.9 mg, 34.6 μmol, 35.2% yield, 97.1% purity, FA) as a white solid.

MS (ESI) m/z: 960.1 [M+H]⁺.

¹H NMR (400 MHz, DMSO) δ=13.05-12.70 (m, 1H), 10.86 (s, 1H), 8.14 (s, 1H), 8.03 (d, J=8.0 Hz, 1H), 7.79 (d, J=8.0 Hz, 1H), 7.63 (d, J=7.2 Hz, 1H), 7.57-7.40 (m, 5H), 7.40-7.33 (m, 2H), 7.10 (t, J=7.6 Hz, 1H), 6.97-6.87 (m, 4H), 6.64 (d, J=7.6 Hz, 1H), 4.98 (s, 2H), 4.31-4.24 (m, 1H), 3.98 (t, J=6.0 Hz, 2H), 3.92 (s, 2H), 3.90 (s, 3H), 3.59-3.53 (m, 2H), 3.01 (t, J=5.6 Hz, 2H), 2.88-2.75 (m, 4H), 2.65-2.57 (m, 6H), 2.32-2.26 (m, 1H), 2.20-2.14 (m, 1H), 1.91 (s, 3H), 1.78-1.72 (m, 2H), 1.55 (s, 2H), 1.49-1.44 (m, 2H), 1.41-1.32 (m, 4H)

### Example 25. Preparation of Compound 138b

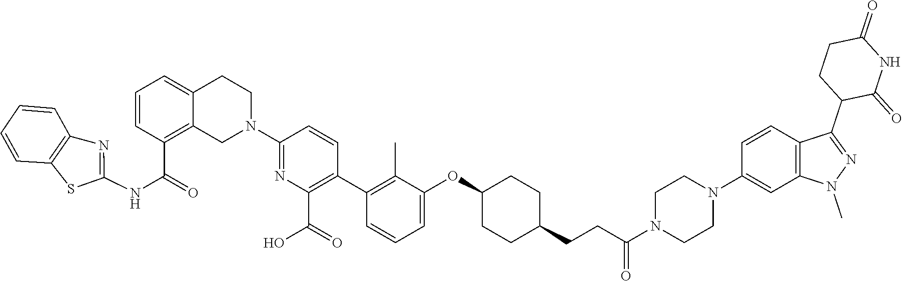 [SMILES: Cc1c(O[C@H]2CC[C@@H](CCC(=O)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4s)-4-(3-(4-(3-(2,6-dioxopiperidin-3-yl)-1-methyl-1H-indazol-6-yl)piperazin-1-yl)-3-oxopropyl)cyclohexyl)oxy)-2-methylphenyl)picolinic acid

### Step A. Procedure for Preparation of tert-butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-6-yl]piperazin-1-yl]-3-oxo-propyl]cyclohexoxy]-2-methyl-phenyl]pyridine-2-carboxylate

To a solution of 3-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]cyclohexyl]propanoic acid (150 mg, 200.83 μmol, 1 equiv.), 3-(1-methyl-6-piperazin-1-yl-indazol-3-yl)piperidine-2,6-dione (87.68 mg, 240.99 μmol, 1.2 equiv., HCl) in DMF (2 mL) was added HATU (91.63 mg, 240.99 μmol, 1.2 equiv.) and DIEA (77.87 mg, 602.48 μmol, 104.94 μL, 3 equiv.). The mixture was stirred at 25° C. for 2 hours. The reaction mixture was diluted with water (10 mL) and filtered to give tert-butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-6-yl]piperazin-1-yl]-3-oxo-propyl]cyclohexoxy]-2-methyl-phenyl]pyridine-2-carboxylate (170 mg, crude) as a yellow solid.

MS (ESI) m/z: 529.0 [½M+H]⁺.

### Step B. Procedure for Preparation of 6-(8-(benzo[d]thiazol-2-ylcarbamoyl)-3,4-dihydroisoquinolin-2(1H)-yl)-3-(3-(((1s,4s)-4-(3-(4-(3-(2,6-dioxopiperidin-3-yl)-1-methyl-1H-indazol-6-yl)piperazin-1-yl)-3-oxopropyl)cyclohexyl)oxy)-2-methylphenyl)picolinic acid

To a solution of tert-butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-6-yl]piperazin-1-yl]-3-oxo-propyl]cyclohexoxy]-2-methyl-phenyl]pyridine-2-carboxylate (170 mg, 160.94 μmol, 1 equiv.) in DCM (1 mL) was added TFA (1.54 g, 13.51 mmol, 1 mL, 83.92 equiv.) The mixture was stirred at 25° C. for 16 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4s)-4-(3-(4-(3-(2,6-dioxopiperidin-3-yl)-1-methyl-1H-indazol-6-yl)piperazin-1-yl)-3-oxopropyl)cyclohexyl)oxy)-2-methylphenyl)picolinic acid (21.1 mg, 20.1 μmol, 12.5% yield, 95.1% purity) as a white solid.

MS (ESI) m/z: 500.9 [½M+H]⁺.

¹H NMR (400 MHz, DMSO-d6) δ=13.12-12.29 (m, 2H), 10.85 (s, 1H), 8.03 (d, J=7.6 Hz, 1H), 7.78 (d, J=8.0 Hz, 1H), 7.62 (d, J=6.8 Hz, 1H), 7.52 (d, J=9.2 Hz, 1H), 7.49-7.42 (m, 3H), 7.40-7.32 (m, 2H), 7.10-7.04 (m, 1H), 6.98-6.91 (m, 2H), 6.91-6.86 (m, 2H), 6.60 (d, J=7.6 Hz, 1H), 4.98 (s, 2H), 4.61 (s, 1H), 4.26 (dd, J=5.0, 9.2 Hz, 1H), 3.93-3.90 (m, 2H), 3.89 (s, 3H), 3.61 (d, J=4.8 Hz, 4H), 3.24-3.16 (m, 4H), 3.02 (t, J=5.6 Hz, 2H), 2.65-2.56 (m, 2H), 2.41-2.36 (m, 2H), 2.33-2.27 (m, 1H), 2.19-2.12 (m, 1H), 1.93 (s, 3H), 1.91-1.86 (m, 1H), 1.55 (d, J=1.6 Hz, 4H), 1.50-1.44 (m, 2H), 1.41-1.20 (m, 4H)

### Example 26. Preparation of Compound 141

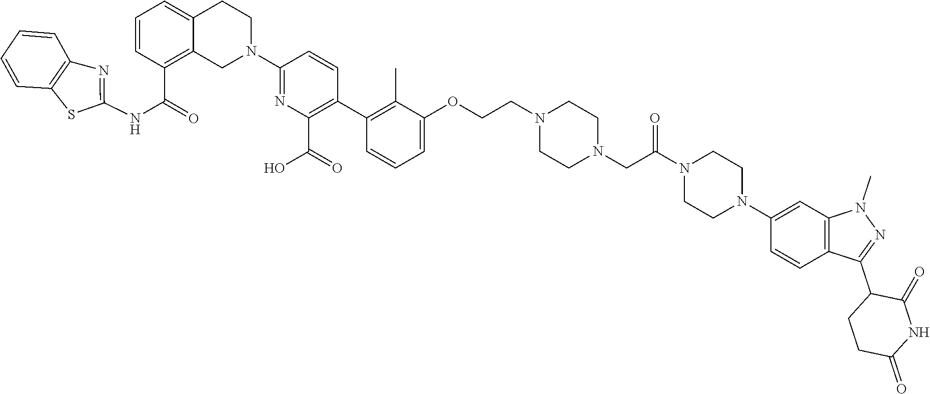 [SMILES: Cc1c(OCCN2CCN(CC(=O)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(2-(4-(2-(4-(3-(2,6-dioxopiperidin-3-yl)-1-methyl-1H-indazol-6-yl)piperazin-1-yl)-2-oxoethyl)piperazin-1-yl)ethoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(2-(4-(2-(4-(3-(2,6-dioxopiperidin-3-yl)-1-methyl-11H-indazol-6-yl)piperazin-1-yl)-2-oxoethyl)piperazin-1-yl)ethoxy)-2-methylphenyl)picolinate

To a solution of 2-(4-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethyl)piperazin-1-yl)acetic acid (100 mg, 131.08 μmol, 1 equiv.) in DMF (1 mL) was added HATU (64.79 mg, 170.40 μmol, 1.3 equiv.), DIEA (50.82 mg, 393.23 μmol, 68.49 μL, 3 equiv.) and 3-(1-methyl-6-piperazin-1-yl-indazol-3-yl)piperidine-2,6-dione (52.46 mg, 144.18 μmol, 1.1 equiv.). The mixture was stirred at 25° C. for 2 hours. The reaction mixture was concentrated under reduced pressure to remove solvent. The residue was purified by prep-TLC (SiO₂, DCM:MeOH=10:1). The compound tert-butyl 6-(8-(benzo[d]thiazol-2-ylcarbamoyl)-3,4-dihydroisoquinolin-2(1H)-yl)-3-(3-(2-(4-(2-(4-(3-(2,6-dioxopiperidin-3-yl)-1-methyl-1H-indazol-6-yl)piperazin-1-yl)-2-oxoethyl)piperazin-1-yl)ethoxy)-2-methylphenyl)picolinate (70 mg, 65.28 μmol, 49.80% yield) was obtained as a white solid.

MS (ESI) m/z: 1072.6 [M+H]⁺.

### Step B. Procedure for Preparation of 6-(8-(benzo[d]thiazol-2-ylcarbamoyl)-3,4-dihydroisoquinolin-2(1H)-yl)-3-(3-(2-(4-(2-(4-(3-(2,6-dioxopiperidin-3-yl)-1-methyl-11H-indazol-6-yl)piperazin-1-yl)-2-oxoethyl)piperazin-1-yl)ethoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(2-(4-(2-(4-(3-(2,6-dioxopiperidin-3-yl)-1-methyl-1H-indazol-6-yl)piperazin-1-yl)-2-oxoethyl)piperazin-1-yl)ethoxy)-2-methylphenyl)picolinate (70 mg, 65.28 μmol, 1 equiv.) in DCM (0.5 mL) was added TFA (770.00 mg, 6.75 mmol, 0.5 mL, 103.44 equiv.). The mixture was stirred at 40° C. for 1 hour. The reaction mixture was concentrated under reduced pressure to remove solvent. The residue was purified by prep-HPLC. The compound 6-(8-(benzo[d]thiazol-2-ylcarbamoyl)-3,4-dihydroisoquinolin-2(1H)-yl)-3-(3-(2-(4-(2-(4-(3-(2,6-dioxopiperidin-3-yl)-1-methyl-1H-indazol-6-yl)piperazin-1-yl)-2-oxoethyl)piperazin-1-yl)ethoxy)-2-methylphenyl)picolinic acid (18.5 mg, 16.8 μmol, 25.7% yield, 96.5% purity) was obtained as a yellow solid.

MS (ESI) m/z: 1016.7 [M+H]⁺.

¹H NMR (400 MHz, DMSO-d₆) δ=10.86 (s, 1H), 8.17 (s, 1H), 8.03 (d, J=8.0 Hz, 1H), 7.79 (d, J=8.0 Hz, 1H), 7.63 (d, J=7.6 Hz, 1H), 7.56-7.32 (m, 7H), 7.09 (t, J=8.0 Hz, 1H), 6.95 (d, J=8.8 Hz, 2H), 6.92-6.87 (m, 2H), 6.64 (d, J=7.6 Hz, 1H), 4.98 (s, 2H), 4.30-4.25 (m, 1H), 4.08-4.04 (m, 2H), 3.92 (s, 2H), 3.90 (s, 3H), 3.74 (s, 2H), 3.62 (d, J=2.0 Hz, 2H), 3.28-3.25 (m, 4H), 3.19 (s, 4H), 3.04 (d, J=5.2 Hz, 2H), 2.72 (t, J=5.6 Hz, 2H), 2.63-2.56 (m, 6H), 2.46 (s, 2H), 2.32-2.25 (m, 1H), 2.19-2.12 (m, 1H), 1.89 (s, 3H)

### Example 27. Preparation of Compound 144

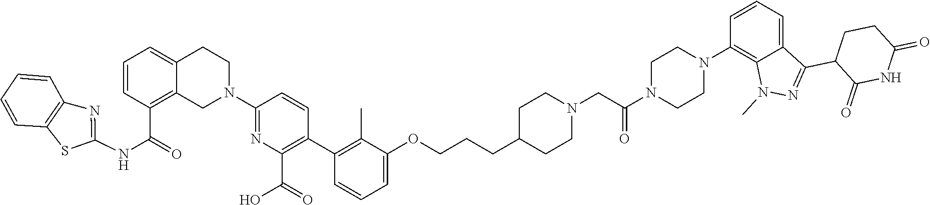 [SMILES: Cc1c(OCCCC2CCN(CC(=O)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[1-[2-[4-[3-(2,6-dioxo-3-piperidyl)-1-methyl-indazol-7-yl]piperazin-1-yl]-2-oxo-ethyl]-4-piperidyl]propoxy]-2-methyl-phenyl]pyridine-2-carboxylic acid

### Step A. Procedure for Preparation of tert-butyl 6-[8-(1,3-benzothiazol-2-ylcarbamoyl)-3,4-dihydro-1H-isoquinolin-2-yl]-3-[3-[3-[1-[2-[4-[3-(2,6-dioxo-3-piperidyl)-1-methyl-indazol-7-yl]piperazin-1-yl]-2-oxo-ethyl]-4-piperidyl]propoxy]-2-methyl-phenyl]pyridine-2-carboxylate

To a solution of 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-1-piperidyl]acetic acid (70 mg, 90.21 μmol, 1 equiv.) in DMF (1 mL) was added 3-(1-methyl-7-piperazin-1-yl-indazol-3-yl)piperidine-2,6-dione (29.53 mg, 90.21 μmol, 1 equiv.), HATU (34.30 mg, 90.21 μmol, 1 equiv.) and DIEA (11.66 mg, 90.21 μmol, 15.71 μL, 1 equiv.). The mixture was stirred at 25° C. for 1 hour. The reaction mixture was concentrated under reduced pressure to give the crude product tert-butyl 6-[8-(1,3-benzothiazol-2-ylcarbamoyl)-3,4-dihydro-1H-isoquinolin-2-yl]-3-[3-[3-[1-[2-[4-[3-(2,6-dioxo-3-piperidyl)-1-methyl-indazol-7-yl]piperazin-1-yl]-2-oxo-ethyl]-4-piperidyl]propoxy]-2-methyl-phenyl]pyridine-2-carboxylate (70 mg, 38.05 μmol, 42.18% yield, 59% purity) as a yellow oil.

MS (ESI) m/z: 543.4 [M12+H]⁺

### Step B. Procedure for Preparation of 6-[8-(1,3-benzothiazol-2-ylcarbamoyl)-3,4-dihydro-1H-isoquinolin-2-yl]-3-[3-[3-[1-[2-[4-[3-(2,6-dioxo-3-piperidyl)-1-methyl-indazol-7-yl]piperazin-1-yl]-2-oxo-ethyl]-4-piperidyl]propoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[3-[1-[2-[4-[3-(2,6-dioxo-3-piperidyl)-1-methyl-indazol-7-yl]piperazin-1-yl]-2-oxo-ethyl]-4-piperidyl]propoxy]-2-methyl-phenyl]pyridine-2-carboxylate (70 mg, 64.50 μmol, 1 equiv.) in TFA (1 mL) and DCM (2 mL). The mixture was stirred at 25° C. for 12 hours. The reaction mixture was concentrated under reduced pressure to remove solvent to give the crude product. The crude product was purified by reverse-phase HPLC to give compound 6-[8-(1,3-benzothiazol-2-ylcarbamoyl)-3,4-dihydro-1H-isoquinolin-2-yl]-3-[3-[3-[1-[2-[4-[3-(2,6-dioxo-3-piperidyl)-1-methyl-indazol-7-yl]piperazin-1-yl]-2-oxo-ethyl]-4-piperidyl]propoxy]-2-methyl-phenyl]pyridine-2-carboxylic acid (15.9 mg, 14.6 μmol, 22.6% yield, 94.1% purity) as a white solid.

MS (ESI) m/z: 1029.1 [M+H]⁺.

¹H NMR (400 MHz, CDCl₃) δ=7.84-7.65 (m, 2H), 7.56 (s, 1H), 7.41-7.28 (m, 4H), 7.24 (t, J=7.6 Hz, 2H), 7.06 (t, J=8.0 Hz, 1H), 6.99-6.84 (m, 3H), 6.73 (d, J=8.4 Hz, 1H), 6.62 (d, J=7.6 Hz, 1H), 5.15 (d, J=5.2 Hz, 2H), 4.59 (d, J=10.8 Hz, 1H), 4.23 (s, 3H), 3.97-3.86 (m, 3H), 3.83-3.72 (m, 1H), 3.37-3.21 (m, 2H), 3.13 (s, 3H), 3.03 (d, J=4.4 Hz, 2H), 2.87-2.72 (m, 4H), 2.64-2.55 (m, 1H), 2.48-2.24 (m, 4H), 2.15-1.98 (m, 4H), 1.84 (s, 3H), 1.75-1.62 (m, 5H), 1.38-1.31 (m, 2H), 1.18 (s, 2H), 0.93-0.63 (m, 1H).

### Example 28. Preparation of Compound 147b

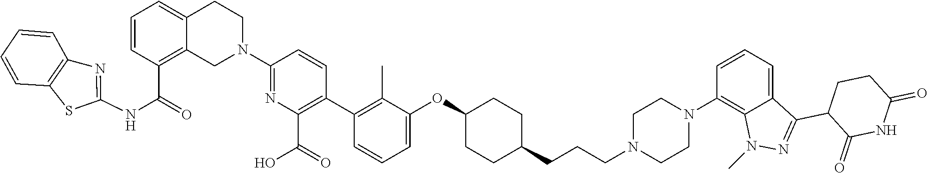 [SMILES: Cc1c(O[C@H]2CC[C@@H](CCCN3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4s)-4-(3-(4-(3-(2,6-dioxopiperidin-3-yl)-1-methyl-1H-indazol-7-yl)piperazin-1-yl)propyl)cyclohexyl)oxy)-2-methylphenyl)picolinic acid

### Step A. Procedure for Preparation of methyl 4-(3-bromo-2-methyl-phenoxy) cyclohexanecarboxylate

To a solution of 3-bromo-2-methyl-phenol (5.91 g, 31.61 mmol, 1 equiv.) in toluene (50 mL) was added methyl 4-hydroxycyclohexanecarboxylate (5 g, 31.61 mmol, 1 equiv.) and 2-(tributyl-λ⁵-phosphanylidene) acetonitrile (9.15 g, 37.93 mmol, 1.2 equiv.) at 25° C. The reaction mixture was degassed and purged with N₂ three times, and then the mixture was stirred at 120° C. for 16 hours under N₂ atmosphere. The reaction mixture was filtered and concentrated under reduced pressure to give a residue. The residue was purified by column chromatography (SiO₂, petroleum ether/ethyl acetate=20/1 to 10/1). The compound methyl 4-(3-bromo-2-methyl-phenoxy) cyclohexanecarboxylate (22.8 g, 43.9 mmol, 69.6% yield, 62.9% purity) was obtained as a colorless oil.

Step B. Procedure for Preparation of [4-(3-bromo-2-methyl-phenoxy) cyclohexyl]methanol

To a solution of methyl 4-(3-bromo-2-methyl-phenoxy) cyclohexanecarboxylate (7.61 g, 14.65 mmol, 62.98% purity, 1 equiv.) in THE (70 mL) was added LiAlH₄ (555.86 mg, 14.65 mmol, 1 equiv.) dropwise at 0° C. under N₂. The reaction mixture was stirred under N₂ at 0° C. for 2 hours. The reaction mixture was quenched with Na₂SO₄·10 H₂O (100 mg) and slowly warmed to 20° C. The mixture was filtered and washed with DCM (50 mL). Then the organic layers were concentrated under reduced pressure to give a residue. The residue was purified by column chromatography (SiO₂, petroleum ether/ethyl acetate=50/1 to 20/1). The compound [4-(3-bromo-2-methyl-phenoxy) cyclohexyl]methanol (8.4 g, 27.7 mmol, 63.0% yield, 98.7% purity) was obtained as a yellow oil.

¹H NMR (400 MHz, CDCl₃-d) δ=7.12 (d, J=8.0 Hz, 1H), 6.98 (t, J=8.0 Hz, 1H), 6.78 (d, J=8.4 Hz, 1H), 4.61-4.54 (m, 1H), 3.52 (d, J=6.0 Hz, 2H), 2.34 (s, 3H), 2.11-2.05 (m, 2H), 1.66-1.61 (m, 3H), 1.59-1.53 (m, 2H), 1.49-1.40 (m, 2H)

### Step C. Procedure for Preparation of 4-(3-bromo-2-methyl-phenoxy) cyclohexanecarbaldehyde

To a solution of DMSO (4.39 g, 56.15 mmol, 4.39 mL, 4 equiv.) in DCM (80 mL) was added dropwise to a solution of oxalyl dichloride (3.56 g, 28.07 mmol, 2.46 mL, 2 equiv.) in DCM (5 mL) at −70° C. under N₂ atmosphere. The mixture was stirred at −70° C. for 30 minutes. After which time [4-(3-bromo-2-methyl-phenoxy) cyclohexyl]methanol (4.2 g, 14.04 mmol, 1 equiv.) in DCM (5 mL) was added dropwise at −70° C. The solution was stirred for 30 minutes at −70° C. Then TEA (8.52 g, 84.22 mmol, 11.72 mL, 6 equiv.) was added into the solution. The solution was stirred at 25° C. for 1 hour under N₂ atmosphere. The reaction mixture was diluted with water 20 mL and extracted with DCM 60 mL (20 mL×3). The combined organic layers were washed with brine (10 mL×2), dried over Na₂SO₄, filtered, and concentrated under reduced pressure to give a residue. The compound 4-(3-bromo-2-methyl-phenoxy) cyclohexanecarbaldehyde (9.33 g, crude) was obtained as a yellow oil.

### Step D. Procedure for Preparation of ethyl (E)-3-[4-(3-bromo-2-methyl-phenoxy) cyclohexyl]prop-2-enoate

To a solution of NaH (282.62 mg, 7.07 mmol, 60% purity, 2.1 equiv.) in THE (10 mL) was added ethyl 2-diethoxyphosphorylacetate (1.51 g, 6.73 mmol, 1.34 mL, 2 equiv.) under 0° C. The mixture was stirred at 0° C. for 4 hours. Then 4-(3-bromo-2-methyl-phenoxy) cyclohexanecarbaldehyde (1 g, 3.36 mmol, 1 equiv.) was added and the mixture was stirred at 25° C. for 12 hours. The reaction mixture was quenched by addition sat. NH₄Cl (10 mL) at 0° C., and then extracted with ethyl acetate 60 mL (20 mL×3). The combined organic layers were washed with brine (20 mL), dried over Na₂SO₄, filtered, and concentrated under reduced pressure to give a residue. The residue was purified by flash silica gel chromatography (Eluent of 0-10% ethyl acetate/petroleum ether). The compound ethyl (E)-3-[4-(3-bromo-2-methyl-phenoxy) cyclohexyl]prop-2-enoate (1.2 g, 2.3 mmol, 68.8% yield, 74% purity) was obtained as a yellow oil.

¹H NMR (400 MHz, CDCl₃-d) δ=7.14 (d, J=8.0 Hz, 1H), 7.01-6.92 (m, 2H), 6.78 (d, J=8.4 Hz, 1H), 5.86-5.79 (m, 1H), 4.60-4.52 (m, 1H), 4.24-4.19 (m, 2H), 2.37-2.32 (m, 3H), 2.31-2.22 (m, 1H), 2.14-2.05 (m, 2H), 1.66-1.62 (m, 4H), 1.36-1.28 (m, 5H)

### Step E. Procedure for Preparation of ethyl 3-[4-(3-bromo-2-methyl-phenoxy) cyclohexyl]propanoate

To a solution of ethyl (E)-3-[4-(3-bromo-2-methyl-phenoxy)cyclohexyl]prop-2-enoate (1.14 g, 3.11 mmol, 1 equiv.) in EtOH (10 mL) was added PtO₂ (141.43 mg, 622.81 μmol, 0.2 equiv.) under N₂ atmosphere. The suspension was degassed and purged with H₂ three times. The mixture was stirred under H₂ (15 Psi) at 25° C. for 40 hours. The reaction mixture was filtered and concentrated under reduced pressure to give a residue. The compound ethyl 3-[4-(3-bromo-2-methyl-phenoxy) cyclohexyl]propanoate (890 mg, crude) was obtained as a yellow oil.

¹H NMR (400 MHz, CDCl₃-d) δ=7.13 (d, J=8.0 Hz, 1H), 6.98 (t, J=8.0 Hz, 1H), 6.78 (d, J=8.0 Hz, 1H), 4.59-4.53 (m, 1H), 4.16 (m, J=7.2 Hz, 2H), 2.39-2.32 (m, 5H), 2.08-1.99 (m, 2H), 1.65-1.54 (m, 5H), 1.44-1.33 (m, 4H), 1.28 (t, J=7.2 Hz, 3H)

SFC (EC2592-447-P1): Column: Chiralpak AD—3 50×4.6 mm I.D., 3 um Mobile phase: Phase A for CO₂, and Phase B for MeOH (0.05% DEA); Gradient elution: MeOH (0.05% DEA) in CO₂ from 5% to 40% Flow rate: 3 mL/min; Detector: PDA; Column Temp: 35 C; Back Pressure: 100 Bar″

### Step F. Procedure for Preparation of 3-[4-(3-bromo-2-methyl-phenoxy) cyclohexyl]propan-1-ol

To a solution of ethyl 3-[4-(3-bromo-2-methyl-phenoxy) cyclohexyl]propanoate (280 mg, 758.21 μmol, 1 equiv.) in THE (3 mL) was added LiAlH₄ (34.53 mg, 909.85 μmol, 1.2 equiv.) dropwise at 0° C. under N₂. The reaction mixture was stirred under N₂ at 0° C. for 2 hours. The reaction mixture was quenched with Na₂SO₄·10 H₂O (10 mg) and slowly warmed to 20° C. The mixture was filtered and washed with DCM (10 mL). Then the organic layers were concentrated under reduced pressure to give a residue. The residue was purified by column chromatography (SiO₂, petroleum ether/ethyl acetate=50/1 to 20/1). The compound 3-[4-(3-bromo-2-methyl-phenoxy) cyclohexyl]propan-1-ol (160 mg, 433.67 μmol, 57.20% yield, 88.7% purity) was obtained as a colorless oil.

Step G. Procedure for Preparation of 3-[4-(3-bromo-2-methyl-phenoxy) cyclohexyl]propanal

A solution of DMSO (135.53 mg, 1.73 mmol, 135.53 μL, 4 equiv.) in DCM (2 mL) was added dropwise to a solution of oxalyl dichloride (110.09 mg, 867.33 μmol, 75.92 μL, 2 equiv.) in DCM (1 mL) at −70° C. under N₂ atmosphere. The mixture was stirred at −70° C. for 30 minutes. After which time 3-[4-(3-bromo-2-methyl-phenoxy) cyclohexyl]propan-1-ol (160 mg, 433.67 μmol, 88.7% purity, 1 equiv.) in DCM (1 mL) was added dropwise at −70° C. The solution was stirred for 30 minutes at −70° C. Then TEA (263.30 mg, 2.60 mmol, 362.17 μL, 6 equiv.) was added into the solution. The solution was stirred at 25° C. for 1 hour under N₂ atmosphere. The reaction mixture was diluted with water (20 mL) and extracted with DCM (10 mL×3). The combined organic layers were washed with brine (10 mL×1), dried over Na₂SO₄, filtered, and concentrated under reduced pressure to give a residue. The compound 3-[4-(3-bromo-2-methyl-phenoxy) cyclohexyl]propanal (215 mg, crude) was obtained as a yellow oil.

### Step H. Procedure for tert-butyl 6-[8-(1,3-benzothiazol-2-ylcarbamoyl)-3,4-dihydro-1H-isoquinolin-2-yl]-3-[2-methyl-3-[4-(3-oxopropyl) cyclohexoxy]phenyl]pyridine-2-carboxylate

tert-Butyl 6-[8-(1,3-benzothiazol-2-ylcarbamoyl)-3,4-dihydro-1H-isoquinolin-2-yl]-3-(4, 4, 5,5-tetramethyl-1, 3,2-dioxaborolan-2-yl) pyridine-2-carboxylate (327.33 mg, 534.37 μmol, 1.1 equiv.), 3-[4-(3-bromo-2-methyl-phenoxy) cyclohexyl]propanal (200 mg, 485.80 μmol, 79% purity, 1 equiv.), KF (84.67 mg, 1.46 mmol, 34.14 μL, 3 equiv.) and [2-(2-aminophenyl) phenyl]palladium (1+); bis (1-adamantyl)-butyl-phosphane; methanesulfonate (70.76 mg, 97.16 mol, 0.2 equiv.) were taken up into a microwave tube in dioxane (4 mL) and H₂O (0.4 mL). The sealed tube was heated at 100° C. for 60 min under microwave. The reaction mixture was filtered and concentrated under reduced pressure to give a residue. The residue was purified by flash silica gel chromatography (eluent of 0˜ 30% ethyl acetate/petroleum ether). The compound tert-butyl 6-[8-(1,3-benzothiazol-2-ylcarbamoyl)-3,4-dihydro-1H-isoquinolin-2-yl]-3-[2-methyl-3-[4-(3-oxopropyl) cyclohexoxy]phenyl]pyridine-2-carboxylate (240 mg, 292.9 μmol, 60.3% yield, 89.2% purity) was obtained as a yellow solid.

MS (ESI) m/z: 731.5 [M+H]⁺.

### Step I. Procedure for tert-butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-7-yl]piperazin-1-yl]propyl]cyclohexoxy]-2-methyl-phenyl]pyridine-2-carboxylate

To a solution of tert-butyl 6-[8-(1,3-benzothiazol-2-ylcarbamoyl)-3,4-dihydro-1H-isoquinolin-2-yl]-3-[2-methyl-3-[4-(3-oxopropyl) cyclohexoxy]phenyl]pyridine-2-carboxylate (140 mg, 191.54 μmol, 1 equiv.) in DCM (2 mL) was added 3-(1-methyl-7-piperazin-1-yl-indazol-3-yl) piperidine-2,6-dione (69.69 mg, 191.54 μmol, 1 equiv., HCl) at 25° C. The mixture was stirred at 25° C. for 16 hours. NaBH(OAc)₃ (121.79 mg, 574.62 μmol, 3 equiv.) was added to the mixture at 25° C. The reaction mixture was stirred at 25° C. for 2 hours. The reaction mixture was filtered and concentrated under reduced pressure to give a residue. The residue was purified by prep-TLC (SiO₂, DCM:MeOH=20:1). The compound tert-butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-7-yl]piperazin-1-yl]propyl]cyclohexoxy]-2-methyl-phenyl]pyridine-2-carboxylate (240 mg, 148.06 mol, 77.3% yield, 64.3% purity) was obtained as a yellow solid.

MS (ESI) m/z: 1042.5 [M+H]⁺.

### Step J. Procedure for Preparation of 6-(8-(benzo[d]thiazol-2-ylcarbamoyl)-3,4-dihydroisoquinolin-2(1H)-yl)-3-(3-(((1r,4s)-4-(3-(4-(3-(2,6-dioxopiperidin-3-yl)-1-methyl-1H-indazol-7-yl)piperazin-1-yl)propyl)cyclohexyl)oxy)-2-methylphenyl)picolinic acid

To a solution of tert-butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-7-yl]piperazin-1-yl]propyl]cyclohexoxy]-2-methyl-phenyl]pyridine-2-carboxylate (240 mg, 148.06 μmol, 64.3% purity, 1 equiv.) in DCM (1.5 mL) and TFA (1.5 mL) was stirred at 25° C. for 16 hours. The reaction mixture was filtered and concentrated under reduced pressure to give a residue. The residue was purified by prep-HPLC. The compound 6-(8-(benzo[d]thiazol-2-ylcarbamoyl)-3,4-dihydroisoquinolin-2(1H)-yl)-3-(3-(((1r,4s)-4-(3-(4-(3-(2,6-dioxopiperidin-3-yl)-1-methyl-1H-indazol-7-yl)piperazin-1-yl)propyl)cyclohexyl)oxy)-2-methylphenyl)picolinic acid (28.9 mg, 28.5 mol, 19.2% yield, 96.9% purity) was obtained as a yellow solid.

MS (ESI) m/z: 986.5 [M+H]⁺.

¹H NMR (400 MHz, DMSO-d₆) δ=10.88 (s, 1H), 8.02 (d, J=8.0 Hz, 1H), 7.79 (d, J=8.0 Hz, 1H), 7.62 (d, J=7.2 Hz, 1H), 7.49-7.42 (m, 3H), 7.40-7.32 (m, 3H), 7.06 (d, J=8.0 Hz, 1H), 7.03-7.00 (m, 2H), 6.96-6.84 (m, 2H), 6.61 (d, J=7.6 Hz, 1H), 4.98 (s, 2H), 4.60 (s, 1H), 4.33 (dd, J=5.2, 9.6 Hz, 1H), 4.22 (s, 3H), 3.91 (t, J=5.6 Hz, 2H), 3.02 (t, J=5.6 Hz, 2H), 2.95-2.85 (m, 3H), 2.69-2.58 (m, 3H), 2.43-2.25 (m, 5H), 2.18 (d, J=5.6 Hz, 1H), 1.97-1.86 (m, 5H), 1.62-1.46 (m, 6H), 1.43-1.10 (m, 6H).

### Example 29. Preparation of Compound 149

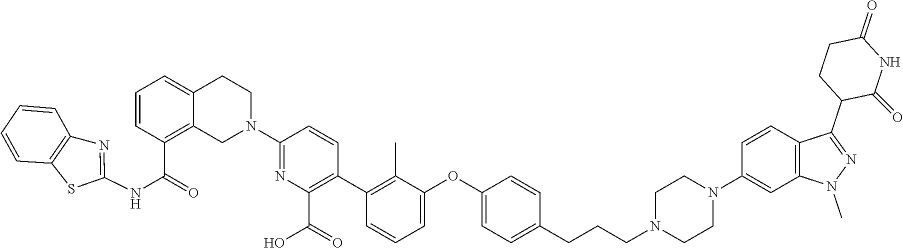 [SMILES: Cc1c(Oc2ccc(CCCN3CCN(c4ccc5c(C6CCC(=O)NC6=O)nn(C)c5c4)CC3)cc2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(4-(3-(4-(3-(2,6-dioxopiperidin-3-yl)-1-methyl-1H-indazol-6-yl)piperazin-1-yl)propyl)phenoxy)-2-methylphenyl)picolinic acid

### Step A. Procedure for Preparation of methyl 3-(4-(3-bromo-2-methylphenoxy)phenyl)propanoate

A mixture of methyl 3-(4-hydroxyphenyl)propanoate (4.64 g, 25.74 mmol, 1.0 equiv.), 1,3-dibromo-2-methyl-benzene (12.87 g, 51.49 mmol, 2.0 equiv.), Cs₂CO₃ (12.58 g, 38.61 mmol, 1.5 equiv.) and 2,2,6,6-tetramethylheptane-3,5-dione (1.19 g, 6.44 mmol, 1.33 mL, 0.25 equiv.) in NMP (50 mL) was degassed and purged with N₂ three times, and then the mixture was added CuI (980.56 mg, 5.15 mmol, 0.2 equiv.) at 120° C. for 12 hours under N₂ atmosphere. The reaction mixture was quenched by addition of water 30 mL at 25° C. and extracted with ethyl acetate (50 mL). The combined organic layers were washed with NaHCO₃ (50 mL×2), dried over Na₂SO₄, filtered, and concentrated under reduced pressure to give a residue. The residue was purified by flash silica gel chromatography (eluent of 0˜5% ethyl acetate/petroleum ether). The compound methyl 3-(4-(3-bromo-2-methylphenoxy)phenyl)propanoate (8.5 g, 24.3 mmol, 94.5% yield) was obtained as a yellow oil.

¹H NMR (400 MHz, CDCl₃) δ=7.24 (d, J=8.0 Hz, 1H), 7.05 (d, J=8.4 Hz, 2H), 6.91 (t, J=8.0 Hz, 1H), 6.78-6.70 (m, 3H), 3.58 (s, 3H), 2.83 (t, J=7.6 Hz, 2H), 2.53 (t, J=7.6 Hz, 2H), 2.24 (s, 3H)

### Step B. Procedure for Preparation of 3-(4-(3-bromo-2-methylphenoxy)phenyl)propan-1-ol

A mixture of methyl 3-[4-(3-bromo-2-methyl-phenoxy)phenyl]propanoate (4.00 g, 11.45 mmol, 1 equiv.) in THE (50 mL) was slowly added LiAlH₄ (521.62 mg, 13.74 mmol, 1.2 equiv.) at 0° C., and then the mixture was stirred at 25° C. for 2 hours. The mixture was poured into ice-water (60 mL) slowly, acidified to pH=4-5, extracted with ethyl acetate 120 mL (40 mL×3), washed with brine 40 mL (20 mL×2), dried over Na₂SO₄, filtered, and concentrated under reduced pressure. The compound 3-(4-(3-bromo-2-methylphenoxy)phenyl)propan-1-ol (3.3 g, 10.3 mmol, 89.7% yield) was obtained as a yellow oil.

### Step C. Procedure for Preparation of 3-(4-(3-bromo-2-methylphenoxy)phenyl)propanal

To a solution of oxalyl dichloride (395.16 mg, 3.11 mmol, 272.53 μL, 2.0 equiv.) in DCM (10 mL) was added the mixture of DMSO (486.48 mg, 6.23 mmol, 486.48 μL, 4.0 equiv.) under −70° C. and stirred for 0.5 hours. 3-[4-(3-bromo-2-methyl-phenoxy)phenyl]propan-1-ol (0.5 g, 1.56 mmol, 1.0 equiv.) in DCM (10 mL) was added into the mixture and stirred for 0.5 hours. TEA (945.09 mg, 9.34 mmol, 1.30 mL, 6 equiv.) was added into the mixture and stirred at −70° C. for 1 hour. The reaction mixture was diluted with citric acid 20 mL and extracted with DCM 45 mL (15 mL×3). The combined organic layers were washed with NaHCO₃ (10 mL×2), dried over Na₂SO₄, filtered, and concentrated under reduced pressure to give a residue. The residue was purified by flash silica gel chromatography (eluent of 0-15% ethyl acetate/petroleum ether). The compound 3-(4-(3-bromo-2-methylphenoxy)phenyl)propanal (250 mg, 783.2 μmol, 50.3% yield) was obtained as a yellow oil.

¹H NMR (400 MHz, DMSO-d₆) δ=9.71 (s, 1H), 7.41 (d, J=8.0 Hz, 1H), 7.24-7.18 (m, 2H), 7.15 (t, J=8.0 Hz, 1H), 6.88-6.81 (m, 3H), 2.82 (d, J=6.4 Hz, 2H), 2.79-2.71 (m, 2H), 2.26 (s, 3H)

### Step D. Procedure for Preparation of tert-butyl 6-(8-(benzo[d]thiazol-2-ylcarbamoyl)-3,4-dihydroisoquinolin-2(1H)-yl)-3-(2-methyl-3-(4-(3-oxopropyl)phenoxy)phenyl)picolinate

A mixture of 3-(4-(3-bromo-2-methylphenoxy)phenyl)propanal (250 mg, 783.23 μmol, 1.0 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (479.76 mg, 783.23 μmol, 1.0 equiv.), K₂CO₃ (1.5 M, 1.57 mL, 3.0 equiv.), and Ad₂nBuP Pd G₃(cataCXium® A Pd G₃) (114.08 mg, 156.65 μmol, 0.2 equiv.) in 1,4-dioxane (3 mL) was degassed and purged with N₂ three times, and then the mixture was stirred at 80° C. for 1 hour under N₂ atmosphere. The reaction mixture was concentrated under reduced pressure to give a residue. The residue was purified by flash silica gel chromatography (eluent of 0-30% ethyl acetate/petroleum ether). The compound tert-butyl 6-(8-(benzo[d]thiazol-2-ylcarbamoyl)-3,4-dihydroisoquinolin-2(1H)-yl)-3-(2-methyl-3-(4-(3-oxopropyl)phenoxy)phenyl)picolinate (200 mg, 219.1 μmol, 27.9% yield, 79.4% purity) was obtained as a yellow oil.

### Step E. Procedure for Preparation of tert-butyl 6-(8-(benzo[d]thiazol-2-ylcarbamoyl)-3,4-dihydroisoquinolin-2(1H)-yl)-3-(3-(4-(3-(4-(3-(2,6-dioxopiperidin-3-yl)-1-methyl-1H-indazol-6-yl)piperazin-1-yl)propyl)phenoxy)-2-methyl phenyl)picolinate

A mixture of tert-butyl 6-(8-(benzo[d]thiazol-2-ylcarbamoyl)-3,4-dihydroisoquinolin-2(1H)-yl)-3-(2-methyl-3-(4-(3-oxopropyl)phenoxy)phenyl)picolinate (100 mg, 108.99 μmol, 79.4% purity, 1.0 equiv.), 3-(1-methyl-6-(piperazin-1-yl)-1H-indazol-3-yl)piperidine-2,6-dione (42.82 mg, 130.78 μmol, 1.2 equiv.), HCOOH (5.24 mg, 108.99 μmol, 1 equiv.), and NaBH(OAc)₃ (34.65 mg, 163.48 μmol, 1.5 equiv.) in DCM (2 mL) was degassed and purged with N₂ three times, and then the mixture was stirred at 25° C. for 16 hours under N₂ atmosphere. The reaction mixture was concentrated under reduced pressure to give a residue. The compound tert-butyl 6-(8-(benzo[d]thiazol-2-ylcarbamoyl)-3,4-dihydroisoquinolin-2(1H)-yl)-3-(3-(4-(3-(4-(3-(2,6-dioxopiperidin-3-yl)-1-methyl-1H-indazol-6-yl)piperazin-1-yl)propyl)phenoxy)-2-methyl phenyl)picolinate (150 mg, crude) was obtained as a yellow oil.

MS (ESI) m/z: 1036.7 [M+H]⁺.

### Step F. Procedure for Preparation of 6-(8-(benzo[d]thiazol-2-ylcarbamoyl)-3,4-dihydroisoquinolin-2(1H)-yl)-3-(3-(4-(3-(4-(3-(2,6-dioxopiperidin-3-yl)-1-methyl-1H-indazol-6-yl)piperazin-1-yl)propyl)phenoxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(3-(4-(3-(4-(3-(2,6-dioxopiperidin-3-yl)-1-methyl-1H-indazol-6-yl)piperazin-1-yl)propyl)phenoxy)-2-methyl phenyl)picolinate (150 mg, 144.75 μmol, 1.0 equiv.), TFA (16.50 mg, 144.75 μmol, 10.72 μL, 1.0 equiv.) in DCM (2 mL) was stirred at 25° C. for 21 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(4-(3-(4-(3-(2,6-dioxopiperidin-3-yl)-1-methyl-1H-indazol-6-yl)piperazin-1-yl)propyl)phenoxy)-2-methylphenyl)picolinic acid (20.9 mg, 20.1 μmol, 13.9% yield, 94.4% purity) as a white solid.

MS (ESI) m/z: 981.2 [M+H]⁺.

¹H NMR (400 MHz, DMSO-d₆) δ=13.05-12.66 (m, 1H), 10.85 (s, 1H), 8.13 (s, 1H), 8.03 (d, J=7.6 Hz, 1H), 7.79 (d, J=8.0 Hz, 1H), 7.62 (d, J=7.6 Hz, 1H), 7.59-7.50 (m, 2H), 7.50-7.42 (m, 3H), 7.40-7.33 (m, 2H), 7.22-7.15 (m, 3H), 7.01 (d, J=8.8 Hz, 1H), 6.90 (dd, J=8.4, 15.2 Hz, 2H), 6.86-6.80 (m, 4H), 4.99 (s, 2H), 4.25 (dd, J=4.8, 8.8 Hz, 1H), 3.93 (s, 2H), 3.89 (s, 3H), 3.03 (t, J=4.8 Hz, 2H), 2.67 (s, 2H), 2.64-2.56 (m, 10H), 2.33 (s, 2H), 2.17 (d, J=5.2 Hz, 2H), 1.90 (s, 3H), 1.83-1.74 (m, 2H)

### Example 30. Preparation of Compound 155

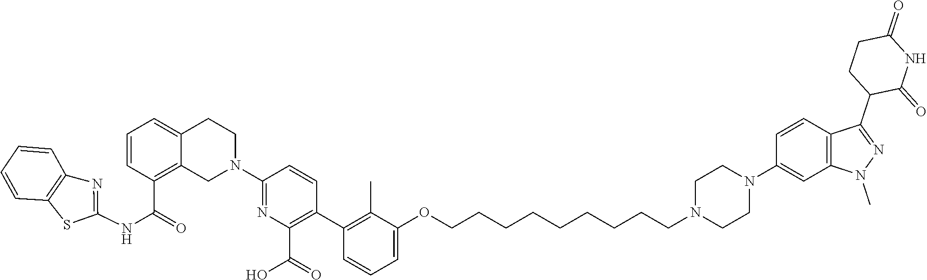 [SMILES: Cc1c(OCCCCCCCCCN2CCN(c3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[9-[4-[3-(2,6-dioxo-3-piperidyl)-1-methyl-indazol-6-yl]piperazin-1-yl]nonoxy]-2-methyl-phenyl]pyridine-2-carboxylic acid

### Step A. Procedure for Preparation of ethyl 9-(3-bromo-2-methyl-phenoxy)nonanoate

To a solution of 3-bromo-2-methyl-phenol (2.0 g, 10.69 mmol, 1 equiv.) in CH₃CN (50 mL) was added K₂CO₃ (4.43 g, 32.07 mmol, 3 equiv.) and ethyl 9-bromononanoate (2.83 g, 10.69 mmol, 1 equiv.). The mixture was stirred at 60° C. for 16 hours. The reaction mixture was concentrated under reduced pressure to give a residue. The residue was purified by column chromatography (SiO₂, petroleum ether/ethyl acetate=1/0 to 10/1) to give compound ethyl 9-(3-bromo-2-methyl-phenoxy)nonanoate (3.2 g, 8.6 mmol, 80.6% yield) as a yellow oil.

¹H NMR (400 MHz, CDCl₃) δ=7.15 (d, J=8.0 Hz, 1H), 7.00 (t, J=8.0 Hz, 1H), 6.78 (d, J=8.0 Hz, 1H), 4.15 (q, J=7.2 Hz, 2H), 3.96 (t, J=6.4 Hz, 2H), 3.43 (t, J=6.8 Hz, 1H), 2.36-2.28 (m, 5H), 1.85-1.77 (m, 2H), 1.65 (t, J=7.2 Hz, 2H), 1.53-1.44 (m, 2H), 1.44-1.31 (m, 8H), 1.28 (t, J=7.2 Hz, 3H).

### Step B. Procedure for Preparation of 9-(3-bromo-2-methyl-phenoxy)nonan-1-ol

A mixture of LiAlH₄ (204.44 mg, 5.39 mmol, 1 equiv.) in THE (25 mL) was degassed and purged with N₂ three times, and ethyl 9-(3-bromo-2-methyl-phenoxy)nonanoate (2.0 g, 5.39 mmol, 1 equiv.) was added at 0° C., and then the mixture was stirred at 0° C. for 2 hours under N₂ atmosphere. The mixture was quenched by water (0.6 mL) at 0° C., and then 15% sodium hydroxide aqueous solution (0.6 mL) and water (0.6 mL), anhydrous sodium sulfate (2.0 g) was added. The mixture was stirred at 25° C. for 30 minutes, filtered, and dissolved in dichloromethane/methanol (4/1, 10 mL). The mixture was stirred at 25° C. for 1 hour, filtered, and concentrated. The residue was purified by column chromatography (SiO₂, petroleum ether/ethyl acetate=1/0 to 4/1) to give the compound 9-(3-bromo-2-methyl-phenoxy)nonan-1-ol (1.5 g, 4.5 mmol, 84.6% yield) as a yellow oil.

¹H NMR (400 MHz, CDCl₃) δ=7.06 (d, J=8.0 Hz, 1H), 6.91 (t, J=8.0 Hz, 1H), 6.68 (d, J=8.0 Hz, 1H), 3.87 (t, J=6.4 Hz, 2H), 3.57 (t, J=6.8 Hz, 2H), 2.24 (s, 3H), 1.75-1.68 (m, 2H), 1.52-1.47 (m, 2H), 1.42-1.34 (m, 4H), 1.27 (s, 6H).

### Step C. Procedure for Preparation of 9-(3-bromo-2-methyl-phenoxy)nonanal

To a solution of DMSO (759.33 mg, 9.72 mmol, 759.33 μL, 4 equiv.) in DCM (20 mL) was added dropwise (COCl)₂ (616.76 mg, 4.86 mmol, 425.35 μL, 2 equiv.) at −70° C. over 10 min. After addition, the mixture was stirred at this temperature for 1 hour, and then 9-(3-bromo-2-methyl-phenoxy)nonan-1-ol (800 mg, 2.43 mmol, 1 equiv.) was added dropwise at 70° C. for 2 hours under N₂ atmosphere. TEA (1.48 g, 14.58 mmol, 2.03 mL, 6 equiv.) was added dropwise at 70° C. for 0.5 hour under N₂. The mixture was stirred at −70° C. for 1 hour. The reaction mixture was extracted with DCM (10 ml) and concentrated under reduced pressure to remove solvent to give a residue. The residue was purified by column chromatography (SiO₂, petroleum ether/ethyl acetate=1/0 to 5/1) to give the compound 9-(3-bromo-2-methyl-phenoxy)nonanal (360 mg, 1.1 mmol, 45.3% yield) as a colorless oil.

¹H NMR (400 MHz, CDCl₃) δ=9.70 (t, J=1.6 Hz, 1H), 7.06 (d, J=8.0 Hz, 1H), 6.91 (t, J=8.0 Hz, 1H), 6.68 (d, J=8.0 Hz, 1H), 3.90-3.84 (m, 2H), 2.39-2.32 (m, 2H), 2.24 (s, 3H), 1.78-1.67 (m, 2H), 1.57 (m, J=7.2 Hz, 2H), 1.42-1.36 (m, 2H), 1.28 (s, 7H), 0.85-0.74 (m, 1H)

### Step D. Procedure for Preparation of tert-butyl 6-(8-(benzo[d]thiazol-2-ylcarbamoyl)-3,4-dihydroisoquinolin-2(1H)-yl)-3-(2-methyl-3-((9-oxononyl)oxy)phenyl)picolinate

9-(3-bromo-2-methyl-phenoxy)nonanal (300 mg, 916.71 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (561.53 mg, 916.71 μmol, 1 equiv.), K₂CO₃ (380.09 mg, 2.75 mmol, 3 equiv.), and Ad₂nBuP Pd G₃(cataCXium® A Pd G₃) (66.76 mg, 91.67 μmol, 0.1 equiv.) were taken up into a microwave tube in 1,4-dioxane (5 mL) and H₂O (1 mL). The sealed tube was heated at 100° C. for 1 hour under microwave. The reaction mixture was concentrated under reduced pressure to remove solution to give a residue. The residue was purified by column chromatography (SiO₂, petroleum ether/ethyl acetate=1/0 to 2/1) to give the compound tert-butyl 6-(8-(benzo[d]thiazol-2-ylcarbamoyl)-3,4-dihydroisoquinolin-2(1H)-yl)-3-(2-methyl-3-((9-oxononyl)oxy)phenyl)picolinate (200 mg, 272.9 μmol, 29.8% yield) as a yellow solid.

MS (ESI) m/z: 733.5 [M+H]⁺

### Step E. Procedure for Preparation of tert-butyl 6-[8-(1,3-benzothiazol-2-ylcarbamoyl)-3,4-dihydro-1H-isoquinolin-2-yl]-3-[3-[9-[4-[3-(2,6-dioxo-3-piperidyl)-1-methyl-indazol-6-yl]piperazin-1-yl]nonoxy]-2-methyl-phenyl]pyridine-2-carboxylate

To a solution of tert-butyl 6-(8-(benzo[d]thiazol-2-ylcarbamoyl)-3,4-dihydroisoquinolin-2(1H)-yl)-3-(2-methyl-3-((9-oxononyl)oxy)phenyl)picolinate (100 mg, 136.44 μmol, 1 equiv.) in EtOH (2 mL) were added 3-(1-methyl-6-piperazin-1-yl-indazol-3-yl)piperidine-2,6-dione (44.67 mg, 136.44 μmol, 1 equiv.), TFA (15.56 mg, 136.44 μmol, 10.10 μL, 1 equiv.). The mixture was stirred at 25° C. for 0.5 hour, and then NaBH(OAc)₃ (86.75 mg, 409.32 μmol, 3 equiv.) was added to the mixture, and the final mixture was stirred at 25° C. for 1 hour. The reaction mixture was concentrated under reduced pressure to give a residue. The residue was purified by column chromatography (SiO₂, DCM:MeOH=1/0 to 20/1) to give the compound tert-butyl 6-[8-(1,3-benzothiazol-2-ylcarbamoyl)-3,4-dihydro-1H-isoquinolin-2-yl]-3-[3-[9-[4-[3-(2,6-dioxo-3-piperidyl)-1-methyl-indazol-6-yl]piperazin-1-yl]nonoxy]-2-methyl-phenyl]pyridine-2-carboxylate (15 mg, 14.4 μmol, 10.5% yield) as a yellow solid.

MS (ESI) m/z: 523.0 [M12+H]⁺

### Step F. Procedure for Preparation of 6-[8-(1,3-benzothiazol-2-ylcarbamoyl)-3,4-dihydro-1H-isoquinolin-2-yl]-3-[3-[9-[4-[3-(2,6-dioxo-3-piperidyl)-1-methyl-indazol-6-yl]piperazin-1-yl]nonoxy]-2-methyl-phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[9-[4-[3-(2,6-dioxo-3-piperidyl)-1-methyl-indazol-6-yl]piperazin-1-yl]nonoxy]-2-methyl-phenyl]pyridine-2-carboxylate (80 mg, 76.61 μmol, 1 equiv.) in TFA (1 mL) and DCM (1 mL) was stirred at 40° C. for 12 hours. The reaction mixture was concentrated under reduced pressure to give a residue. The residue was purified by reverse-phase HPLC to give the compound 6-[8-(1,3-benzothiazol-2-ylcarbamoyl)-3,4-dihydro-1H-isoquinolin-2-yl]-3-[3-[9-[4-[3-(2,6-dioxo-3-piperidyl)-1-methyl-indazol-6-yl]piperazin-1-yl]nonoxy]-2-methyl-phenyl]pyridine-2-carboxylic acid (50.2 mg, 47.0 μmol, 61.3% yield, 92.4% purity) as a white solid.

MS (ESI) m/z: 988.7 [M+H]⁺.

¹H NMR (400 MHz, DMSO-d₆) δ=13.29-12.43 (m, 1H), 10.86 (s, 1H), 8.02 (d, J=8.0 Hz, 1H), 7.78 (d, J=8.0 Hz, 1H), 7.62 (d, J=7.2 Hz, 1H), 7.52 (d, J=8.8 Hz, 1H), 7.49-7.40 (m, 3H), 7.39-7.32 (m, 2H), 7.12-7.06 (m, 1H), 6.98-6.84 (m, 4H), 6.63 (d, J=7.6 Hz, 1H), 4.97 (s, 2H), 4.26 (dd, J=4.8, 9.2 Hz, 1H), 3.96 (t, J=6.4 Hz, 2H), 3.89 (s, 5H), 3.65-3.50 (m, 4H), 3.00 (t, J=5.6 Hz, 2H), 2.97-2.81 (m, 4H), 2.72-2.58 (m, 4H), 2.34-2.26 (m, 1H), 2.20-2.10 (m, 1H), 1.90 (s, 3H), 1.79-1.68 (m, 2H), 1.55 (d, J=2.0 Hz, 2H), 1.45 (d, J=6.8 Hz, 2H), 1.31 (s, 8H).

### Example 31. Preparation of Compound 160a

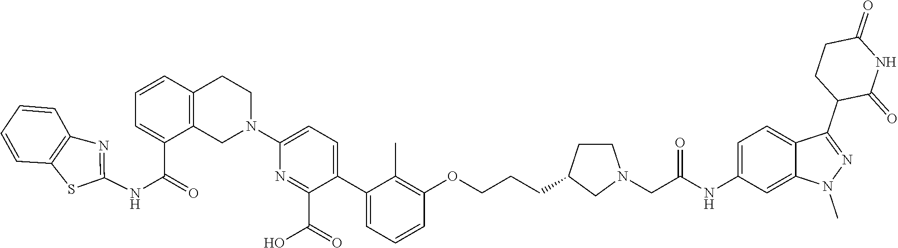 [SMILES: Cc1c(OCCC[C@@H]2CCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[(3R)-1-[2-[[3-(2,6-dioxo-3-piperidyl)-1-methyl-indazol-6-yl]amino]-2-oxo-ethyl]pyrrolidin-3-yl]propoxy]-2-methyl-phenyl]pyridine-2-carboxylic acid

### Step A. Procedure for Preparation of tert-butyl (3S)-3-[(E)-3-ethoxy-3-oxo-prop-1-enyl]pyrrolidine-1-carboxylate

To a solution of tert-butyl (3R)-3-formylpyrrolidine-1-carboxylate (4 g, 20.08 mmol, 1 equiv.) in THE (60 mL) was added NaH (1.20 g, 30.11 mmol, 60% purity, 1.5 equiv.) stirred at 0° C. for 10 min under N₂ atmosphere, then added ethyl 2-diethoxyphosphorylacetate (9.00 g, 40.15 mmol, 7.97 mL, 2 equiv.) the mixture was stirred at 0° C. for 2 hours under N₂ atmosphere. The reaction mixture was quenched by slowly adding water (30 mL) at 0° C. The resulting mixture was then extracted with ethyl acetate (30 mL×3). The combined organic layers were washed with water (30 mL×3), filtered, and concentrated under reduced pressure to give a residue. The residue was purified by flash silica gel chromatography (Eluent of 0˜28% ethyl acetate/petroleum ether) to give tert-butyl (3S)-3-[(E)-3-ethoxy-3-oxo-prop-1-enyl]pyrrolidine-1-carboxylate (4.5 g, 16.7 mmol, 83.2% yield) as a white solid.

¹H NMR (400 MHz, DMSO-d₆) δ=6.88-6.82 (m, 1H), 5.95-5.91 (m, 1H), 5.77-5.74 (m, 1H), 4.14-4.09 (m, 2H), 3.48-3.43 (m, 1H), 3.38-3.29 (m, 2H), 3.25-3.16 (m, 1H), 3.08-2.94 (m, 2H), 1.80-1.70 (m, 1H), 1.39 (s, 9H), 1.22-1.19 (t, J=7.1 Hz, 3H).

### Step B. Procedure for Preparation of tert-butyl (3R)-3-(3-ethoxy-3-oxo-propyl) pyrrolidine-1-carboxylate

To a solution of tert-butyl (3S)-3-[(E)-3-ethoxy-3-oxo-prop-1-enyl]pyrrolidine-1-carboxylate (4.5 g, 16.71 mmol, 1 equiv.) in EtOH (60 mL) was added PtO₂ (1.14 g, 5.01 mmol, 0.3 equiv.) in EtOH 5 mL under H₂ atmosphere (15 Psi). The mixture was stirred at 25° C. for 12 hours. The mixture was filtered and washed with EtOH 15 mL (5 mL×3), concentrated under reduced pressure to give a residue. The residue used in the next step without purification. The compound tert-butyl (3R)-3-(3-ethoxy-3-oxo-propyl) pyrrolidine-1-carboxylate (3.8 g, crude) was obtained as a white solid.

¹H NMR (400 MHz, DMSO-d₆) δ=4.08-4.03 (m, 2H), 3.47-3.38 (m, 2H), 3.30-3.29 (m, 1H), 3.18-3.08 (m, 1H), 2.80-2.74 (m, 1H), 2.34-2.30 (m, 2H), 2.11-2.00 (m, 1H), 1.95-1.90 (m, 1H), 1.66-1.53 (m, 2H), 1.39 (s, 9H), 1.24-1.16 (m, 3H).

### Step C. Procedure for Preparation of tert-butyl (3R)-3-(3-hydroxypropyl)pyrrolidine-1-carboxylate

To a solution of tert-butyl (3R)-3-(3-ethoxy-3-oxo-propyl) pyrrolidine-1-carboxylate (3.5 g, 12.90 mmol, 1 equiv.) in THE (40 mL) was added LiAlH₄ (489.55 mg, 12.90 mmol, 1 equiv.). The mixture was stirred at 0° C. for 2 hours. The reaction mixture was quenched by addition water (0.49 mL) and 15% NaOH (0.49 mL) at 0° C., and then diluted with water (5 mL) and extracted with ethyl acetate (15 mL×3). The combined organic layers were washed with water (15 mL×3), concentrated under reduced pressure to give a residue. The residue was purified by flash silica gel chromatography (Eluent of 0˜40% ethyl acetate/petroleum ether) to give tert-butyl (3R)-3-(3-hydroxypropyl)pyrrolidine-1-carboxylate (2.6 g, 11.34 mmol, 87.9% yield) as a white solid.

MS (ESI) m/z: 130.5 [M+H-100]⁺

### Step D. Procedure for Preparation of tert-butyl (3R)-3-[3-(3-bromo-2-methyl-phenoxy)propyl]pyrrolidine-1-carboxylate

A mixture of tert-butyl (3R)-3-(3-hydroxypropyl)pyrrolidine-1-carboxylate (2.6 g, 11.34 mmol, 1 equiv.), 3-bromo-2-methyl-phenol (2.54 g, 13.61 mmol, 1.2 equiv.), 2-(tributyl-λ5-phosphanylidene)acetonitrile (3.28 g, 13.61 mmol, 1.2 equiv.) in toluene (30 mL) was degassed and purged with N₂ three times, and then the mixture was stirred at 120° C. for 12 hours under N₂ atmosphere. The reaction mixture was concentrated under reduced pressure to give a residue. The residue was purified by flash silica gel chromatography (Eluent of 0˜17% ethyl acetate/petroleum ether) to give tert-butyl (3R)-3-[3-(3-bromo-2-methyl-phenoxy)propyl]pyrrolidine-1-carboxylate (3.4 g, 8.0 mmol, 70.7% yield, 94% purity) as a white solid.

MS (ESI) m/z: 300.2 [M+H-100]⁺

### Step E. Procedure for Preparation of (3R)-3-[3-(3-bromo-2-methyl-phenoxy) propyl]pyrrolidine

To a solution of tert-butyl (3R)-3-[3-(3-bromo-2-methyl-phenoxy)propyl]pyrrolidine-1-carboxylate (3.4 g, 8.54 mmol, 1 equiv.) in HCl/dioxane (4 M) (10 mL). The mixture was stirred at 25° C. for 1 hour. The reaction mixture was concentrated under reduced pressure to give a residue. The residue was used in the next step without purification. The compound (3R)-3-[3-(3-bromo-2-methyl-phenoxy) propyl] pyrrolidine (2.8 g, crude) was obtained as a white solid.

### Step F. Procedure for Preparation of ethyl 2-[(3R)-3-[3-(3-bromo-2-methyl-phenoxy)propyl]pyrrolidin-1-yl]acetate

A mixture of (3R)-3-[3-(3-bromo-2-methyl-phenoxy)propyl]pyrrolidine (2.8 g, 8.37 mmol, 1 equiv., HCl), ethyl 2-bromoacetate (1.12 g, 6.69 mmol, 740.22 μL, 0.8 equiv.), K₂CO₃ (3.47 g, 25.10 mmol, 3 equiv.) in CH₃CN (40 mL) was degassed and purged with N₂ three times, and then the mixture was stirred at 40° C. for 1 hour under N₂ atmosphere. The reaction mixture was concentrated under reduced pressure to give a residue. The residue was purified by flash silica gel chromatography (Eluent of 0-17% ethyl acetate/petroleum ether) to give a crude. The crude was purified by prep-SFC to give ethyl 2-[(3R)-3-[3-(3-bromo-2-methyl-phenoxy)propyl]pyrrolidin-1-yl]acetate (2 g, 4.8 mmol, 57.8% yield, 93% purity) as a white solid.

MS (ESI) m/z: 384.2 [M+H]⁺

### Step G. Procedure for Preparation of tert-butyl 6-[8-(1,3-benzothiazol-2-ylcarbamoyl)-3,4-dihydro-1H-isoquinolin-2-yl]-3-[3-[3-[(3R)-1-(2-ethoxy-2-oxo-ethyl)pyrrolidin-3-yl]propoxy]-2-methyl-phenyl]pyridine-2-carboxylate

Ethyl 2-[(3R)-3-[3-(3-bromo-2-methyl-phenoxy)propyl]pyrrolidin-1-yl]acetate (300 mg, 780.63 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (525.99 mg, 858.69 μmol, 1.1 equiv.), Ad₂nBuP Pd G₃(cataCXium® A Pd G₃) (113.70 mg, 156.13 μmol, 0.2 equiv.) and K₂CO₃ (1.5 M, 780.63 μL, 1.5 equiv.) were taken up into a microwave tube in 1,4-dioxane (4 mL). The sealed tube was heated at 100° C. for 1 hour under microwave. The reaction mixture was concentrated under reduced pressure to give a residue. The residue was purified by flash silica gel chromatography (Eluent of 0˜55% ethyl acetate/petroleum ether) to give tert-butyl 6-[8-(1,3-benzothiazol-2-ylcarbamoyl)-3,4-dihydro-1H-isoquinolin-2-yl]-3-[3-[3-[(3R)-1-(2-ethoxy-2-oxo-ethyl)pyrrolidin-3-yl]propoxy]-2-methyl-phenyl]pyridine-2-carboxylate (450 mg, 569.6 μmol, 72.9% yield) as a white solid.

MS (ESI) m/z: 790.4 [M+H]⁺

### Step H. Procedure for Preparation of 2-[(3R)-3-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy] propyl] pyrrolidin-1-yl] acetic acid

A mixture of tert-butyl 6-[8-(1,3-benzothiazol-2-ylcarbamoyl)-3,4-dihydro-1H-isoquinolin-2-yl]-3-[3-[3-[(3R)-1-(2-ethoxy-2-oxo-ethyl)pyrrolidin-3-yl]propoxy]-2-methyl-phenyl]pyridine-2-carboxylate (450 mg, 569.64 μmol, 1 equiv.), LiOH·H₂O (71.71 mg, 1.71 mmol, 3 equiv.) in THE (8 mL) and H₂O (2 mL) was degassed and purged with N₂ three times, and then the mixture was stirred at 25° C. for 3 hours under N₂ atmosphere. The reaction mixture was concentrated under reduced pressure to remove THF, then added HCl (1 mol/L) and adjusted pH to 5. The mixture was filtered and used in the next step without purification. The compound 2-[(3R)-3-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy] propyl] pyrrolidin-1-yl] acetic acid (360 mg, crude) was obtained as a white solid.

MS (ESI) m/z: 762.8 [M+H]⁺

### Step I. Procedure for Preparation of tert-butyl 6-[8-(1,3-benzothiazol-2-ylcarbamoyl)-3,4-dihydro-1H-isoquinolin-2-yl]-3-[3-[3-[(3R)-1-[2-[[3-(2,6-dioxo-3-piperidyl)-1-methyl-indazol-6-yl]amino]-2-oxo-ethyl]pyrrolidin-3-yl]propoxy]-2-methyl-phenyl]pyridine-2-carboxylate

A mixture of 2-[(3R)-3-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy] propyl] pyrrolidin-1-yl]acetic acid (120 mg, 157.50 μmol, 1 equiv.), 3-(6-amino-1-methyl-indazol-3-yl)piperidine-2,6-dione (48.81 mg, 188.99 μmol, 1.2 equiv.), DIPEA (61.07 mg, 472.49 μmol, 82.30 μL, 3 equiv.), HATU (59.88 mg, 157.50 μmol, 1.5 equiv.) in DMF (3 mL) was degassed and purged with N₂ three times, and then the mixture was stirred at 25° C. for 3 hours under N₂ atmosphere. The reaction mixture was quenched by slow addition of water 30 mL at 0° C., and then extracted with ethyl acetate (30 mL×3). The combined organic layers were washed with water (30 mL×3), filtered, and concentrated under reduced pressure to give a residue. The residue was used in the next step without purification. The compound tert-butyl 6-[8-(1,3-benzothiazol-2-ylcarbamoyl)-3,4-dihydro-1H-isoquinolin-2-yl]-3-[3-[3-[(3R)-1-[2-[[3-(2,6-dioxo-3-piperidyl)-1-methyl-indazol-6-yl]amino]-2-oxo-ethyl]pyrrolidin-3-yl]propoxy]-2-methyl-phenyl]pyridine-2-carboxylate (150 mg, crude) was obtained as a white solid.

MS (ESI) m/z: 1002.6 [M+H]⁺

### Step J. Procedure for Preparation of 6-[8-(1,3-benzothiazol-2-ylcarbamoyl)-3,4-dihydro-1H-isoquinolin-2-yl]-3-[3-[3-[(3R)-1-[2-[[3-(2,6-dioxo-3-piperidyl)-1-methyl-indazol-6-yl]amino]-2-oxo-ethyl]pyrrolidin-3-yl]propoxy]-2-methyl-phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[3-[3-[(3R)-1-[2-[[3-(2,6-dioxo-3-piperidyl)-1-methyl-indazol-6-yl]amino]-2-oxo-ethyl]pyrrolidin-3-yl]propoxy]-2-methyl-phenyl]pyridine-2-carboxylate (150 mg, 149.67 μmol, 1 equiv.) in TFA (3 mL) and DCM (3 mL) was degassed and purged with N₂ three times, and then the mixture was stirred at 40° C. for 2 hours under N₂ atmosphere. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[3-[(3R)-1-[2-[[3-(2,6-dioxo-3-piperidyl)-1-methyl-indazol-6-yl]amino]-2-oxo-ethyl]pyrrolidin-3-yl]propoxy]-2-methyl-phenyl]pyridine-2-carboxylic acid (54.2 mg, 56.2 μmol, 37.5% yield, 98.0% purity) as a white solid.

MS (ESI) m/z: 946.7 [M+H]⁺.

¹H NMR (400 MHz, DMSO-d₆) δ=13.36-12.29 (m, 1H), 10.88 (s, 1H), 10.00 (s, 1H), 8.14 (s, 1H), 8.06-8.00 (m, 2H), 7.78 (d, J=8.0 Hz, 1H), 7.62 (d, J=8.4 Hz, 2H), 7.49-7.42 (m, 3H), 7.39-7.32 (m, 2H), 7.19 (d, J=9.2 Hz, 1H), 7.12-7.05 (m, 1H), 6.95 (d, J=9.2 Hz, 1H), 6.88 (d, J=8.4 Hz, 1H), 6.62 (d, J=7.2 Hz, 1H), 4.99-4.96 (m, 2H), 4.33-4.30 (m, 9.8 Hz, 1H), 4.02-3.93 (m, 3H), 3.92-3.90 (m, 4H), 3.05-2.95 (m, 5H), 2.84-2.79 (m, 2H), 2.66-2.62 (m, 2H), 2.35-2.32 (m, 2H), 2.24-2.15 (m, 2H), 2.05-1.98 (m, 1H), 1.90 (s, 3H), 1.77-1.69 (m, 2H), 1.57-1.51 (m, 2H), 1.47-1.40 (m, 1H).

### Example 32. Preparation of Compound 160b

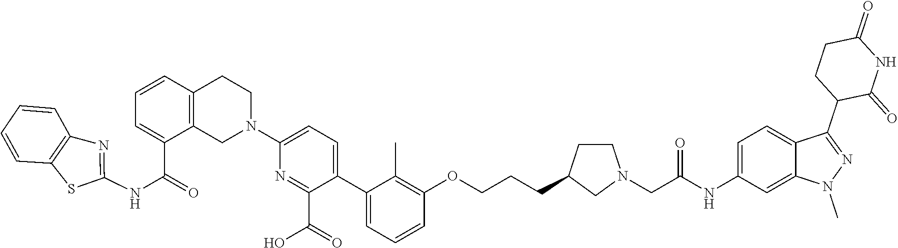 [SMILES: Cc1c(OCCC[C@H]2CCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-((3S)-1-(2-((3-(2,6-dioxopiperidin-3-yl)-1-methyl-1H-indazol-6-yl)amino)-2-oxoethyl)pyrrolidin-3-yl)propoxy)-2-methylphenyl)picolinic acid

### Step A. Procedure for Preparation of (R,E)-tert-butyl 3-(3-ethoxy-3-oxoprop-1-en-1-yl)pyrrolidine-1-carboxylate

A mixture of NaH (1.69 g, 42.16 mmol, 60% purity, 2.1 equiv.) in THE (40 mL) was purged with N₂ for three times. To the mixture was slowly added ethyl 2-(diethoxyphosphoryl)acetate (9.00 g, 40.15 mmol, 7.97 mL, 2 equiv.) at 0° C., and then the mixture was stirred for 0.5 hours under N₂ atmosphere. To the resulting solution was added (S)-tert-butyl 3-formylpyrrolidine-1-carboxylate (4 g, 20.08 mmol, 1 equiv.) at 0° C. The solution was warm to 25° C. and stirred for 16 hours under N₂ atmosphere. The reaction mixture was quenched by addition saturated NH₄Cl (40 mL) under 0° C. The mixture was extracted with ethyl acetate (20 mL×3). The combined organic layers were washed with brine (20 mL×2), dried over Na₂SO₄, filtered, and concentrated under reduced pressure to give (R,E)-tert-butyl 3-(3-ethoxy-3-oxoprop-1-en-1-yl)pyrrolidine-1-carboxylate (2.4 g, 9.0 mmol, 45.1% yield) as a white oil.

¹H NMR (400 MHz, DMSO-d₆) δ=6.85 (dd, J=7.6, 15.6 Hz, 1H), 5.93 (dd, J=0.8, 15.6 Hz, 1H), 4.11 (q, J=7.2 Hz, 2H), 3.45 (dd, J=7.2, 10.0 Hz, 1H), 3.39-3.31 (m, 3H), 3.26-3.15 (m, 1H), 2.07-1.96 (m, 1H), 1.82-1.67 (m, 1H), 1.39 (s, 9H), 1.24-1.17 (m, 3H)

### Step B. Procedure for Preparation of (S)-tert-butyl 3-(3-ethoxy-3-oxopropyl)pyrrolidine-1-carboxylate

A mixture of (R,E)-tert-butyl 3-(3-ethoxy-3-oxoprop-1-en-1-yl)pyrrolidine-1-carboxylate (2.44 g, 9.06 mmol, 1 equiv.) PtO₂ (1.44 g, 6.34 mmol, 0.7 equiv.) in EtOH (25 mL) was degassed and purged with H₂ three times, and then the mixture was stirred at 25° C. for 15 hours under H₂ (15 Psi) atmosphere. The mixture was filtered by EtOH (30 mL), and the filtrate was concentrated under reduced pressure to give (S)-tert-butyl 3-(3-ethoxy-3-oxopropyl)pyrrolidine-1-carboxylate (1.41 g, crude) as a black oil (The filter cake was immersed in EtOH).

¹H NMR (400 MHz, DMSO-d₆) δ=4.05 (q, J=7.2 Hz, 2H), 3.44-3.28 (m, 4H), 2.34-2.27 (m, 2H), 2.11-1.99 (m, 1H), 1.97-1.86 (m, 1H), 1.59 (dq, J=3.2, 7.6 Hz, 2H), 1.49-1.41 (m, 1H), 1.38 (s, 9H), 1.18 (t, J=7.2 Hz, 3H)

### Step C. Procedure for Preparation of (S)-tert-butyl 3-(3-hydroxypropyl)pyrrolidine-1-carboxylate

A mixture of (S)-tert-butyl 3-(3-ethoxy-3-oxopropyl)pyrrolidine-1-carboxylate (1.41 g, 5.20 mmol, 1 equiv.) in THE (25 mL) was slowly added LiAlH₄ (197.20 mg, 5.20 mmol, 1 equiv.) at 0° C., then the mixture was stirred at 25° C. for 5 hours. The mixture was quenched by 6.5 g Na₂SO₄·10 H₂O. The solution was poured onto ice-water (30 mL) slowly and acidified to pH=4-5 with 1 M HCl. The resulting mixture was extracted with EtOAc 60 mL (20 mL×3), washed with brine 40 mL (20 mL×2), dried by sodium sulfate, filtered, and concentrated under reduced pressure to give (S)-tert-butyl 3-(3-hydroxypropyl)pyrrolidine-1-carboxylate (1.08 g, 4.07 mmol, 78.31% yield, 86.4% purity) as a white oil.

### Step D. Procedure for Preparation of (S)-tert-butyl 3-(3-(3-bromo-2-methylphenoxy)propyl)pyrrolidine-1-carboxylate

A mixture of (S)-tert-butyl 3-(3-hydroxypropyl)pyrrolidine-1-carboxylate (1.08 g, 4.71 mmol, 1 equiv.), 3-bromo-2-methylphenol (1.06 g, 5.65 mmol, 1.2 equiv.), 2-(tributyl-λ⁵-phosphanylidene)acetonitrile (1.36 g, 5.65 mmol, 1.2 equiv.) in toluene (10 mL) was degassed and purged with N₂ three times, and then the mixture was stirred at 120° C. for 18 hours under N₂ atmosphere. The mixture concentrated under reduced pressure to give a residue. The residue was purified by flash silica gel chromatography (Eluent of 0˜7% ethyl acetate/petroleum ether) to give (S)-tert-butyl 3-(3-(3-bromo-2-methylphenoxy)propyl)pyrrolidine-1-carboxylate (1.05 g, 2.48 mmol, 52.61% yield, 94.0% purity) as a yellow oil.

### Step E. Procedure for Preparation of (S)-3-(3-(3-bromo-2-methylphenoxy)propyl)pyrrolidine

A mixture of (S)-tert-butyl 3-(3-(3-bromo-2-methylphenoxy)propyl)pyrrolidine-1-carboxylate (905 mg, 2.27 mmol, 1 equiv.) in HCl/EtOAc (10 mL) was stirred at 25° C. for 16 hours. The mixture was concentrated under the reduced pressure to give (S)-3-(3-(3-bromo-2-methylphenoxy)propyl)pyrrolidine (685 mg, 2.2 mmol, 99.2% yield, 98.2% purity) a colorless oil.

MS (ESI) m/z: 300.1 [M+H]⁺

### Step F. Procedure for Preparation of (S)-ethyl 2-(3-(3-(3-bromo-2-methylphenoxy)propyl)pyrrolidin-1-yl)acetate

A mixture of (S)-3-(3-(3-bromo-2-methylphenoxy)propyl)pyrrolidine (685 mg, 2.30 mmol, 1 equiv.), ethyl 2-bromoacetate (383.60 mg, 2.30 mmol, 254.04 μL, 1 equiv.), K₂CO₃ (952.40 mg, 6.89 mmol, 3 equiv.) in CH₃CN (1 mL) was degassed and purged with N₂ three times, and then the mixture was stirred at 60° C. for 6 hours under N₂ atmosphere. The mixture concentrated under the reduced pressure to give a residue. The residue was purified by flash silica gel chromatography (Eluent of 0˜75% ethyl acetate/petroleum ether) to give (S)-ethyl 2-(3-(3-(3-bromo-2-methylphenoxy)propyl)pyrrolidin-1-yl)acetate (903 mg, 2.30 mmol, 99.94% yield, 97.7% purity) as a blue oil.

MS (ESI) m/z: 384.1 [M+H]⁺

¹H NMR (400 MHz, DMSO-d₆) δ=7.16-7.12 (m, 1H), 7.11-7.06 (m, 1H), 6.95 (d, J=8.0 Hz, 1H), 4.10 (q, J=7.2 Hz, 2H), 3.96 (t, J=6.4 Hz, 2H), 3.47 (d, J=4.4 Hz, 2H), 2.96 (t, J=8.4 Hz, 1H), 2.81 (q, J=7.6 Hz, 1H), 2.73-2.66 (m, 1H), 2.40-2.31 (m, 1H), 2.23 (s, 3H), 2.20-2.11 (m, 1H), 2.01-1.91 (m, 1H), 1.77-1.65 (m, 2H), 1.54-1.45 (m, 2H), 1.44-1.34 (m, 1H), 1.19 (t, J=7.2 Hz, 3H)

### Step H. Procedure for Preparation of (S)-tert-butyl 6-(8-(benzo[d]thiazol-2-ylcarbamoyl)-3,4-dihydroisoquinolin-2(1H)-yl)-3-(3-(3-(1-(2-methoxy-2-oxoethyl)pyrrolidin-3-yl)propoxy)-2-methylphenyl)picolinate

(S)-ethyl 2-(3-(3-(3-bromo-2-methylphenoxy)propyl)pyrrolidin-1-yl)acetate (314 mg, 817.06 μmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (550.53 mg, 898.76 μmol, 1.1 equiv.), Ad₂nBuP Pd G₃ (119.01 mg, 163.41 μmol, 0.2 equiv.), KF (1.5 M, 817.06 μL, 1.5 equiv.) in dioxane (10 mL) and were taken up into a microwave tube in dioxane (10 mL), The sealed tube was heated at 100° C. for 2 hours under microwave. The reaction mixture was partitioned between H₂O (15 mL) and ethyl acetate 60 mL (20 mL×3). The organic phase was separated, washed with aqueous NaCl 45 mL (15 mL×3), dried over (Na₂SO₄), filtered, and concentrated under reduced pressure to give a residue. The residue was purified by flash silica gel chromatography (Eluent of 0˜74% ethyl acetate/petroleum ether) to give (S)-tert-butyl 6-(8-(benzo[d]thiazol-2-ylcarbamoyl)-3,4-dihydroisoquinolin-2(1H)-yl)-3-(3-(3-(1-(2-methoxy-2-oxoethyl)pyrrolidin-3-yl)propoxy)-2-methylphenyl)picolinate (368 mg, 465.83 μmol, 57.01% yield) as a yellow solid.

MS (ESI) m/z: 790.3 [M+H]⁺

### Step L Procedure for preparation of (S)-2-(3-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)pyrrolidin-1-yl)acetic acid

A mixture of (S)-tert-butyl 6-(8-(benzo[d]thiazol-2-ylcarbamoyl)-3,4-dihydroisoquinolin-2(1H)-yl)-3-(3-(3-(1-(2-methoxy-2-oxoethyl)pyrrolidin-3-yl)propoxy)-2-methylphenyl)picolinate (368 mg, 465.83 μmol, 1 equiv.), LiOH·H₂O (1 M, 1.40 mL, 3 equiv.) in THE (6 mL) was stirred at 25° C. for 6 hours. The mixture was concentrated and diluted with H₂O (2 mL), then the pH of the mixture was adjusted to 3 with 1 M HCl. Then the mixture was filtered and concentrated to give (S)-2-(3-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)pyrrolidin-1-yl)acetic acid (313 mg, 390.67 μmol, 83.86% yield, 95.1% purity) as a white solid.

MS (ESI) m/z: 762.6 [M+H]⁺

### Step J. Procedure for Preparation of tert-butyl 6-(8-(benzo[d]thiazol-2-ylcarbamoyl)-3,4-dihydroisoquinolin-2(1H)-yl)-3-(3-(3-((3S)-1-(2-((3-(2,6-dioxopiperidin-3-yl)-1-methyl-11H-indazol-6-yl)amino)-2-oxoethyl)pyrrolidin-3-yl)propoxy)-2-methylphenyl)picolinate

A mixture of (S)-2-(3-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)pyrrolidin-1-yl)acetic acid (93 mg, 122.06 μmol, 1 equiv.), 3-(6-amino-1-methyl-1H-indazol-3-yl)piperidine-2,6-dione (37.83 mg, 146.47 μmol, 1.2 equiv.) in pyridine (2 mL) was added EDCI (35.10 mg, 183.09 μmol, 1.5 equiv.). The resulting mixture was stirred at 25° C. for 15 hours. The reaction mixture was partitioned between H₂O (10 mL) and DCM (10 mL×3). The organic phase was separated, washed with aqueous NaCl (10 mL×2), dried over (Na₂SO₄), filtered, and concentrated under reduced pressure to give tert-butyl 6-(8-(benzo[d]thiazol-2-ylcarbamoyl)-3,4-dihydroisoquinolin-2(1H)-yl)-3-(3-(3-((3S)-1-(2-((3-(2,6-dioxopiperidin-3-yl)-1-methyl-1H-indazol-6-yl)amino)-2-oxoethyl)pyrrolidin-3-yl)propoxy)-2-methylphenyl)picolinate (113 mg, crude) as a red solid.

MS (ESI) m/z: 1002.4 [M+H]⁺

### Step K. Procedure for Preparation of 6-(8-(benzo[d]thiazol-2-ylcarbamoyl)-3,4-dihydroisoquinolin-2(1H)-yl)-3-(3-(3-((3S)-1-(2-((3-(2,6-dioxopiperidin-3-yl)-1-methyl-11H-indazol-6-yl)amino)-2-oxoethyl)pyrrolidin-3-yl)propoxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(3-(3-((3S)-1-(2-((3-(2,6-dioxopiperidin-3-yl)-1-methyl-1H-indazol-6-yl)amino)-2-oxoethyl)pyrrolidin-3-yl)propoxy)-2-methylphenyl)picolinate (113 mg, 112.75 μmol, 1 equiv.) in DCM (3 mL) and TFA (1 mL) was stirred at 40° C. for 69 hours under N₂ atmosphere. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(3-((3S)-1-(2-((3-(2,6-dioxopiperidin-3-yl)-1-methyl-1H-indazol-6-yl)amino)-2-oxoethyl)pyrrolidin-3-yl)propoxy)-2-methylphenyl)picolinic acid (57.2 mg, 55.8 μmol, 49.5% yield, 92.4% purity) as a yellow solid

MS (ESI) m/z: 946.7 [M+H]⁺

¹H NMR (400 MHz, DMSO-d₆) δ=12.92-12.81 (m, 1H), 10.89 (s, 1H), 10.12 (d, J=8.0 Hz, 1H), 8.05-8.01 (m, 1H), 7.98 (s, 1H), 7.79 (d, J=7.6 Hz, 1H), 7.69 (d, J=8.8 Hz, 1H), 7.63 (d, J=6.4 Hz, 1H), 7.49-7.42 (m, 3H), 7.40-7.33 (m, 2H), 7.14-7.08 (m, 2H), 6.97 (d, J=8.8 Hz, 1H), 6.89 (d, J=8.4 Hz, 1H), 6.64 (d, J=7.6 Hz, 1H), 4.98 (s, 2H), 4.33 (dd, J=5.2, 10.0 Hz, 1H), 4.27 (dd, J=3.2, 4.4 Hz, 2H), 3.98 (t, J=6.0 Hz, 2H), 3.93 (s, 3H), 3.92-3.88 (m, 2H), 3.76-3.57 (m, 2H), 3.03 (t, J=5.6 Hz, 2H), 2.74-2.59 (m, 4H), 2.36-2.30 (m, 2H), 2.27-2.00 (m, 3H), 1.91 (s, 3H), 1.81-1.74 (m, 2H), 1.66-1.59 (m, 2H)

### Example 33. Preparation of Compound 162a

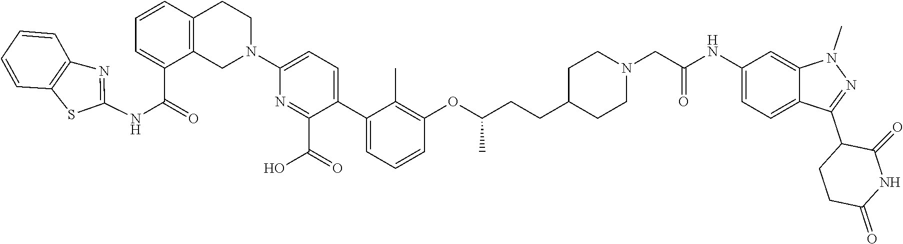 [SMILES: Cc1c(O[C@@H](C)CCC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-H-isoquinolin-2-yl]-3-[3-[(1 S)-3-[1-[2-[[3-(2,6-dioxo-3-piperidyl)-1-methyl-indazol-6-yl]amino]-2-oxo-ethyl]-4-piperidyl]-1-methyl-propoxy]-2-methyl-phenyl]pyridine-2-carboxylic acid

### Step A. Procedure for Preparation of tert-butyl 4-[3-[methoxy(methyl)amino]-3-oxo-propyl]piperidine-1-carboxylate

To a solution of 3-(1-tert-butoxycarbonyl-4-piperidyl)propanoic acid (4 g, 15.54 mmol, 1 equiv.) and N-methoxymethanamine (2.27 g, 23.32 mmol, 1.5 equiv., HCl) in DMF (10 mL) was added DIEA (6.03 g, 46.63 mmol, 8.12 mL, 3 equiv.) and HATU (8.87 g, 23.32 mmol, 1.5 equiv.). The mixture was stirred at 25° C. for 12 hours. The reaction mixture was quenched by addition H₂O (30 mL), and then extracted with ethyl acetate (30 mL×3). The combined organic layers were washed with brine (30 mL×3), dried over anhydrous sodium sulfate, filtered and concentrated under reduced pressure to give a residue. The residue was purified by column chromatography (SiO₂, petroleum ether/ethyl acetate=10/1 to 1/1) to give compound tert-butyl 4-[3-[methoxy(methyl)amino]-3-oxo-propyl]piperidine-1-carboxylate (4.5 g, 14.9 mmol, 96.3% yield) as a yellow oil.

¹H NMR (400 MHz, CDCl₃) δ=3.69 (s, 3H), 3.18 (s, 3H), 2.68 (t, J=12.0 Hz, 2H), 2.45 (t, J=8.0 Hz, 2H), 1.69 (s, 1H), 1.66 (s, 2H), 1.63-1.56 (m, 2H), 1.45 (s, 11H), 1.18-1.05 (m, 2H).

### Step B. Procedure for Preparation of tert-butyl 4-(3-oxobutyl)piperidine-1-carboxylate

A mixture of tert-butyl 4-[3-[methoxy(methyl)amino]-3-oxo-propyl]piperidine-1-carboxylate (4.5 g, 14.98 mmol, 1 equiv.) in THE (50 mL) was degassed and purged with N₂ three times, and then MeMgBr (3 M, 29.96 mL, 6 equiv.) was added dropwise to the mixture and stirred at 0° C. for 4 hours under N₂ atmosphere. The reaction mixture was quenched by addition saturated ammonium chloride (50 mL) at 0° C., and then diluted with water (50 mL) and extracted with CH₂Cl₂ (50 mL×3). The combined organic layers were washed with brine (50 mL×3), dried over anhydrous sodium sulfate, filtered, and concentrated under reduced pressure to give compound tert-butyl 4-(3-oxobutyl)piperidine-1-carboxylate (3.23 g, 12.65 mmol, 84.44% yield) as a white solid.

¹H NMR (400 MHz, DMSO-d₆) δ=3.90 (d, J=11.2 Hz, 2H), 2.64-2.63 (m, 2H), 2.44 (t, J=7.2 Hz, 2H), 2.07 (s, 3H), 1.59 (d, J=12.4 Hz, 2H), 1.41-1.35 (m, 12H), 0.98-0.86 (m, 2H).

### Step C. Procedure for Preparation of tert-butyl 4-(3-hydroxybutyl)piperidine-1-carboxylate

To a solution of tert-butyl 4-(3-oxobutyl)piperidine-1-carboxylate (2 g, 7.83 mmol, 1 equiv.) in EtOH (20 mL) was added NaBH₄ (296.32 mg, 7.83 mmol, 1 equiv.). The mixture was stirred at 0° C. for 1 hour. The reaction mixture was concentrated under reduced pressure to remove EtOH, and then diluted with EtOAc 10 mL and saturated ammonium chloride solution 10 m1 and extracted with EtOAc (10 mL×3). The combined organic layers were washed with brine (10 mL×3), dried over anhydrous sodium sulfate, filtered, and concentrated under reduced pressure to give compound tert-butyl 4-(3-hydroxybutyl)piperidine-1-carboxylate (1.8 g, 6.99 mmol, 89.30% yield) as a white solid.

¹H NMR (400 MHz, DMSO-d₆) δ=4.31 (d, J=4.4 Hz, 1H), 3.90 (d, J=12.4 Hz, 2H), 3.58-3.48 (m, 1H), 2.73-2.61 (m, 2H), 1.61 (d, J=12.4 Hz, 2H), 1.38 (s, 12H), 1.33-1.22 (m, 4H), 1.02 (d, J=6.0 Hz, 3H).

### Step D. Procedure for Preparation of tert-butyl 4-[(3R)-3-hydroxybutyl]piperidine-1-carboxylate

This page was used for purification. This tert-butyl 4-(3-hydroxybutyl)piperidine-1-carboxylate (3 g, 11.66 mmol, 1 equiv.) was separated by SFC to give tert-butyl 4-[(3S)-3-hydroxybutyl]piperidine-1-carboxylate (1.2 g, 4.66 mmol, 40.00% yield) as a colorless oil and tert-butyl 4-[(3R)-3-hydroxybutyl]piperidine-1-carboxylate (1.08 g, 4.20 mmol, 36.00% yield) as a pink oil.

¹H NMR (400 MHz, DMSO-d₆) δ=4.29 (d, J=4.4 Hz, 1H), 3.90 (d, J=12.4 Hz, 2H), 3.61-3.46 (m, 1H), 2.66-2.65 (m, 2H), 1.61 (d, J=12.8 Hz, 2H), 1.38 (s, 9H), 1.35-1.23 (m, 4H), 1.21-1.11 (m, 1H), 1.02 (d, J=6.0 Hz, 3H), 0.99-0.85 (m, 2H).

### Step E. Procedure for Preparation of tert-butyl 4-[(3S)-3-(3-bromo-2-methyl-phenoxy)butyl]piperidine-1-carboxylate

To a solution of tert-butyl 4-[(3R)-3-hydroxybutyl]piperidine-1-carboxylate (1.1 g, 4.27 mmol, 1 equiv.) and 3-bromo-2-methyl-phenol (959.27 mg, 5.13 mmol, 1.2 equiv.) in toluene (10 mL) was added 2-(tributyl-λ5-phosphanylidene)acetonitrile (1.55 g, 6.41 mmol, 1.5 equiv.). The mixture was stirred at 120° C. for 12 hours. The reaction mixture was concentrated under reduced pressure to give a residue. The residue was purified by column chromatography (SiO₂, petroleum ether/ethyl acetate=0/1 to 10/1) to give compound tert-butyl 4-[(3S)-3-(3-bromo-2-methyl-phenoxy)butyl]piperidine-1-carboxylate (1.86 g, crude) as a colorless oil.

¹H NMR (400 MHz, CDCl₃) δ=7.13 (d, J=8.0 Hz, 1H), 6.98 (t, J=8.0 Hz, 1H), 6.76 (d, J=8.4 Hz, 1H), 4.08 (d, J=12.4 Hz, 2H), 2.68 (t, J=11.2 Hz, 2H), 2.30 (s, 3H), 1.70-1.58 (m, 4H), 1.46 (s, 9H), 1.29 (d, J=6.4 Hz, 5H), 1.15-1.07 (m, 2H), 0.94-0.81 (m, 2H).

### Step F. Procedure for Preparation of 4-[(3S)-3-(3-bromo-2-methyl-phenoxy)butyl]piperidine

A solution of tert-butyl 4-[(3S)-3-(3-bromo-2-methyl-phenoxy)butyl]piperidine-1-carboxylate (1.86 g, 4.36 mmol, 1 equiv.) in HCl/EtOAc (4 M, 10 mL, 9.17 equiv.) was stirred at 25° C. for 1 hour. The reaction mixture was concentrated under reduced pressure to give a residue. The residue was purified by column chromatography (SiO₂, petroleum ether/ethyl acetate=1/1 to DCM:MeOH=10:1) to give compound 4-[(3S)-3-(3-bromo-2-methyl-phenoxy)butyl]piperidine (1.55 g, crude) as a light yellow oil.

### Step G. Procedure for Preparation of ethyl 2-[4-[(3S)-3-(3-bromo-2-methyl-phenoxy)butyl]-1-piperidyl]acetate

To a solution of 4-[(3S)-3-(3-bromo-2-methyl-phenoxy)butyl]piperidine (0.8 g, 2.45 mmol, 1 equiv.) and ethyl 2-bromoacetate (409.48 mg, 2.45 mmol, 271.18 μL, 1 equiv.) in CH₃CN (10 mL) was added K₂CO₃ (1.02 g, 7.36 mmol, 3 equiv.). The mixture was stirred at 60° C. for 12 hours. The reaction mixture was filtered and concentrated under reduced pressure to give a residue. The residue was purified by column chromatography (SiO₂, petroleum ether/ethyl acetate=0/1 to DCM: MeOH=10/1) to give compound ethyl 2-[4-[(3S)-3-(3-bromo-2-methyl-phenoxy)butyl]-1-piperidyl]acetate (575 mg, 1.3 mmol, 56.8% yield) as a yellow oil.

MS (ESI) m/z: 414.1 [M+H]⁺

¹H NMR (400 MHz, CDCl₃) δ=7.13 (d, J=8.0 Hz, 1H), 6.98 (t, J=8.0 Hz, 1H), 6.76 (d, J=8.0 Hz, 1H), 4.35-4.27 (m, 1H), 4.24-4.15 (m, 2H), 3.21 (s, 2H), 2.94 (d, J=10.8 Hz, 2H), 2.30 (s, 3H), 2.18-2.15 (m, 2H), 1.75-1.62 (m, 4H), 1.40-1.25 (m, 11H).

### Step H. Procedure for Preparation of tert-butyl 6-[8-(1,3-benzothiazol-2-ylcarbamoyl)-3,4-dihydro-1H-isoquinolin-2-yl]-3-[3-[(1S)-3-[1-(2-ethoxy-2-oxo-ethyl)-4-piperidyl]-1-methyl-propoxy]-2-methyl-phenyl]pyridine-2-carboxylate

Ethyl 2-[4-[(3S)-3-(3-bromo-2-methyl-phenoxy)butyl]-1-piperidyl]acetate (575 mg, 1.39 mmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (854.14 mg, 1.39 mmol, 1 equiv.), [2-(2-aminophenyl)phenyl]palladium(1+); bis(1-adamantyl)-butyl-phosphane; methanesulfonate (203.10 mg, 278.88 μmol, 0.2 equiv.), and KF (1.5 M, 2.79 mL, 3 equiv.) were taken up into a microwave tube in dioxane (5 mL). The sealed tube was heated at 100° C. for 60 minutes under microwave. The reaction mixture was concentrated under reduced pressure to give a residue. The residue was purified by column chromatography (SiO₂, petroleum ether/ethyl acetate=0/1 to 1/1) to give compound tert-butyl 6-[8-(1,3-benzothiazol-2-ylcarbamoyl)-3,4-dihydro-1H-isoquinolin-2-yl]-3-[3-[(1S)-3-[1-(2-ethoxy-2-oxo-ethyl)-4-piperidyl]-1-methyl-propoxy]-2-methyl-phenyl]pyridine-2-carboxylate (0.616 g, 753.03 μmol, 54.00% yield) as a yellow oil.

MS (ESI) m/z: 818.6 [M+H]⁺

¹H NMR (400 MHz, DMSO-d₆) δ=12.86 (s, 1H), 8.02 (d, J=7.6 Hz, 1H), 7.78 (d, J=8.0 Hz, 1H), 7.60 (d, J=7.6 Hz, 1H), 7.49-7.42 (m, 3H), 7.41-7.31 (m, 2H), 7.13-7.04 (m, 1H), 6.97-6.88 (m, 2H), 6.54 (d, J=7.2 Hz, 1H), 4.97 (s, 2H), 4.42-4.41 (m, 1H), 4.07-4.01 (m, 4H), 3.87 (t, J=5.2 Hz, 2H), 3.30 (s, 4H), 3.13 (s, 2H), 3.03 (t, J=6.0 Hz, 3H), 2.77 (d, J=9.2 Hz, 2H), 2.12-2.04 (m, 2H), 1.99 (s, 4H), 1.85 (s, 3H), 1.65-1.53 (m, 4H), 1.00 (s, 9H).

### Step L Procedure for preparation of 2-[4-[(3S)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]butyl]-1-piperidyl]acetic acid

To a solution of tert-butyl 6-[8-(1,3-benzothiazol-2-ylcarbamoyl)-3,4-dihydro-1H-isoquinolin-2-yl]-3-[3-[(1S)-3-[1-(2-ethoxy-2-oxo-ethyl)-4-piperidyl]-1-methyl-propoxy]-2-methyl-phenyl]pyridine-2-carboxylate (0.616 g, 753.03 μmol, 1 equiv.) in THE (3 mL) and H₂O (3 mL) was added LiOH·H₂O (158.00 mg, 3.77 mmol, 5 equiv.). The mixture was stirred at 25° C. for 2 hours. The reaction mixture was quenched by addition water 10 mL, and then filtered and concentrated under reduced pressure to give compound 2-[4-[(3S)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]butyl]-1-piperidyl]acetic acid (640 mg, crude) as a yellow solid.

MS (ESI) m/z: 790.4 [M+H]⁺

### Step J. Procedure for Preparation of tert-butyl 6-[8-(1,3-benzothiazol-2-ylcarbamoyl)-3,4-dihydro-1H-isoquinolin-2-yl]-3-[3-[(1S)-3-[1-[2-[[3-(2,6-dioxo-3-piperidyl)-1-methyl-indazol-6-yl]amino]-2-oxo-ethyl]-4-piperidyl]-1-methyl-propoxy]-2-methyl-phenyl]pyridine-2-carboxylate

To a solution of 2-[4-[(3S)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]butyl]-1-piperidyl]acetic acid (60 mg, 75.95 μmol, 1 equiv.) and 3-(6-amino-1-methyl-indazol-3-yl)piperidine-2,6-dione (23.54 mg, 91.14 μmol, 1.2 equiv.) in DMF (1.0 mL) was added HATU (43.32 mg, 113.93 μmol, 1.5 equiv.) and DIEA (49.08 mg, 379.76 μmol, 66.15 μL, 5 equiv.). The mixture was stirred at 25° C. for 12 hours. The reaction mixture was quenched by addition of water (10 mL), filtered and concentrated under reduced pressure to give tert-butyl 6-[8-(1,3-benzothiazol-2-ylcarbamoyl)-3,4-dihydro-1H-isoquinolin-2-yl]-3-[3-[(1S)-3-[1-[2-[[3-(2,6-dioxo-3-piperidyl)-1-methyl-indazol-6-yl]amino]-2-oxo-ethyl]-4-piperidyl]-1-methyl-propoxy]-2-methyl-phenyl]pyridine-2-carboxylate (120 mg, crude) as a pink solid.

MS (ESI) m/z: 1030.9 [M+H]⁺

### Step K. Procedure for Preparation of 6-[8-(1,3-benzothiazol-2-ylcarbamoyl)-3,4-dihydro-1H-isoquinolin-2-yl]-3-[3-[(1S)-3-[1-[2-[[3-(2,6-dioxo-3-piperidyl)-1-methyl-indazol-6-yl]amino]-2-oxo-ethyl]-4-piperidyl]-1-methyl-propoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[(1S)-3-[1-[2-[[3-(2,6-dioxo-3-piperidyl)-1-methyl-indazol-6-yl]amino]-2-oxo-ethyl]-4-piperidyl]-1-methyl-propoxy]-2-methyl-phenyl]pyridine-2-carboxylate (120 mg, 116.48 μmol, 1 equiv.) in DCM (1 mL) was added TFA (13.28 mg, 116.48 μmol, 8.62 μL, 1 equiv.). The mixture was stirred at 25° C. for 12 hours. The reaction mixture was concentrated under reduced pressure to give a residue. The residue was purified by prep-HPLC to give compound 6-[8-(1,3-benzothiazol-2-ylcarbamoyl)-3,4-dihydro-1H-isoquinolin-2-yl]-3-[3-[(1S)-3-[1-[2-[[3-(2,6-dioxo-3-piperidyl)-1-methyl-indazol-6-yl]amino]-2-oxo-ethyl]-4-piperidyl]-1-methyl-propoxy]-2-methyl-phenyl]pyridine-2-carboxylic acid (24.5 mg, 24.0 μmol, 20.6% yield, 95.2% purity) as a white solid.

MS (ESI) m/z: 974.4 [M+H]⁺

¹H NMR (400 MHz, DMSO-d₆) δ=10.89 (s, 1H), 9.84 (s, 1H), 8.06-7.99 (m, 2H), 7.78 (d, J=7.6 Hz, 1H), 7.62 (d, J=8.4 Hz, 2H), 7.49-7.41 (m, 3H), 7.40-7.31 (m, 2H), 7.23-7.18 (m, 1H), 7.11-7.04 (m, 1H), 6.97-6.85 (m, 2H), 6.61 (d, J=7.6 Hz, 1H), 4.97 (s, 2H), 4.46-4.37 (m, 1H), 4.35-4.28 (m, 1H), 3.91 (s, 5H), 3.12 (s, 2H), 3.02 (br t, J=5.6 Hz, 2H), 2.86 (d, J=11.2 Hz, 2H), 2.69-2.59 (m, 2H), 2.38-2.27 (m, 2H), 2.19-2.12 (m, 2H), 1.88 (s, 3H), 1.72-1.59 (m, 4H), 1.26-1.23 (m, 8H).

### Example 34. Preparation of Compound 163

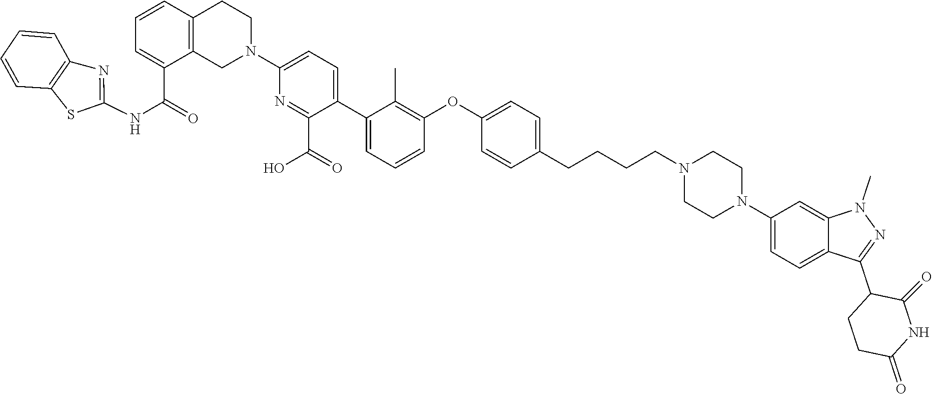 [SMILES: Cc1c(Oc2ccc(CCCCN3CCN(c4ccc5c(C6CCC(=O)NC6=O)nn(C)c5c4)CC3)cc2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(4-(4-(4-(3-(2,6-dioxopiperidin-3-yl)-1-methyl-1H-indazol-6-yl)piperazin-1-yl)butyl)phenoxy)-2-methylphenyl)picolinic acid

### Step A. Procedure for Preparation of ethyl 4-(4-hydroxyphenyl)butanoate

A mixture of 4-(4-hydroxyphenyl)butanoic acid (900 mg, 4.99 mmol, 1 equiv.), SOCl₂ (891.28 mg, 7.49 mmol, 543.47 μL, 1.5 equiv.), in EtOH (10 mL) was degassed and purged with N₂ three times, and then the mixture was stirred at 90° C. for 10 h under N₂ atmosphere. The mixture was concentrated under reduced pressure to give the crude compound ethyl 4-(4-hydroxyphenyl)butanoate (1.24 g, crude) as a yellow oil. The residue was used for next step without other purification.

¹H NMR (400 MHz, DMSO-d₆) δ=9.15 (s, 1H), 6.96 (d, J=8.4 Hz, 2H), 6.67 (d, J=8.4 Hz, 2H), 4.04 (q, J=7.2 Hz, 2H), 2.46 (t, J=7.6 Hz, 2H), 2.25 (t, J=7.6 Hz, 2H), 1.76 (quin, J=7.6 Hz, 2H), 1.17 (t, J=7.2 Hz, 3H)

### Step B. Procedure for Preparation of ethyl 4-(4-(3-bromo-2-methylphenoxy)phenyl)butanoate

A mixture of 1,3-dibromo-2-methyl-benzene (4.18 g, 16.71 mmol, 3 equiv.), ethyl 4-(4-hydroxyphenyl)butanoate (1.16 g, 5.57 mmol, 1 equiv.), CuI (530.42 mg, 2.79 mmol, 0.5 equiv.), Cs₂CO₃ (2.18 g, 6.68 mmol, 1.2 equiv.) and 2,2,6,6-tetramethylheptane-3,5-dione (256.62 mg, 1.39 mmol, 286.72 μL, 0.25 equiv.) in NMP (12.4 mL) was degassed and purged with N₂ three times, and then the mixture was stirred at 120° C. for 16 h under N₂ atmosphere. The mixture was diluted with H₂O (10 mL) and extracted with ethyl acetate (5 mL×3). The combined organic layers were filtered and concentrated under reduced pressure to give a residue. The residue was purified by flash silica gel chromatography (Eluent of 0˜ 5% ethyl acetate/petroleum ether) to give ethyl 4-(4-(3-bromo-2-methylphenoxy)phenyl)butanoate (1.6 g, 4.2 mmol, 76.1% yield) as a colorless oil

¹H NMR (400 MHz, DMSO-d₆) δ=7.41 (d, J=8.0 Hz, 1H), 7.20-7.12 (m, 3H), 6.89-6.83 (m, 3H), 4.04 (q, J=7.2 Hz, 2H), 2.56 (t, J=7.6 Hz, 2H), 2.31-2.27 (m, 2H), 2.26 (s, 3H), 1.80 (m, J=7.6 Hz, 2H), 1.17 (t, J=7.2 Hz, 3H)

### Step C. Procedure for Preparation of 4-(4-(3-bromo-2-methylphenoxy)phenyl)butan-1-ol

To a solution of ethyl 4-[4-(3-bromo-2-methylphenoxy)phenyl]butanoate (600 mg, 1.59 mmol, 1 equiv.) in THF (6 mL) was added LiAlH₄ (54.33 mg, 1.43 mmol, 0.9 equiv.) under 0° C. at N₂ atmosphere, The mixture was stirred at 0° C. for 2 h. The reaction mixture was quenched by addition Na₂SO₄·10H₂O (1 g) under 0° C. and N₂ atmosphere, and then filtered and concentrated under reduced pressure to give a residue. The residue was used for next step without other purification. The compound 4-(4-(3-bromo-2-methylphenoxy)phenyl)butan-1-ol (861 mg, crude) was obtained as a yellow oil.

¹H NMR (400 MHz, DMSO-d₆) δ=7.43 (d, J=7.8 Hz, 1H), 7.21-7.15 (m, 3H), 6.89-6.86 (m, 3H), 4.40 (t, J=5.2 Hz, 1H), 3.45-3.40 (m, 2H), 2.57 (t, J=7.6 Hz, 2H), 2.29 (s, 3H), 1.63-1.56 (m, 2H), 1.48-1.41 (m, 2H)

### Step D. Procedure for Preparation of 4-(4-(3-bromo-2-methylphenoxy)phenyl)butanal

To a solution of DMSO (802.70 mg, 10.27 mmol, 802.70 μL, 4 equiv.) in DCM (8 mL) was added dropwise to a solution of (COCl)₂ (651.98 mg, 5.14 mmol, 449.64 μL, 2 equiv.) in DCM (4 mL) at −70° C. under N₂ atmosphere. The mixture was stirred at −70° C. for 1 h. After which time 4-(4-(3-bromo-2-methylphenoxy)phenyl)butan-1-ol (861 mg, 2.57 mmol, 1 equiv.) in DCM (2 mL) was added dropwise at −70° C. The solution was stirred for 1 h at −70° C. Then TEA (1.56 g, 15.41 mmol, 2.14 mL, 6 equiv.) was added into the solution. The solution was stirred at −70° C. for 0.5 h under N₂ atmosphere. The mixture was diluted with H₂O (10 mL) and extracted with DCM (5 mL×3). The combined organic layers were filtered and concentrated under reduced pressure to give the crude compound 4-(4-(3-bromo-2-methylphenoxy)phenyl)butanal (624 mg, 1.8 mmol, 72.9% yield) was obtained as a yellow oil, and it was used for next step without other purification.

¹H NMR (400 MHz, DMSO-d₆) δ=9.68 (s, 1H), 7.42 (d, J=8.0 Hz, 1H), 7.22-7.15 (m, 3H), 6.90-6.84 (m, 3H), 2.59-2.54 (m, 2H), 2.44 (t, J=7.2 Hz, 2H), 2.27 (s, 3H), 1.82 (m, J=7.5 Hz, 2H)

### Step E. Procedure for Preparation of tert-butyl 6-(8-(benzo[d]thiazol-2-ylcarbamoyl)-3,4-dihydroisoquinolin-2(1H)-yl)-3-(2-methyl-3-(4-(4-oxobutyl)phenoxy)phenyl)picolinate

A mixture of 4-[4-(3-bromo-2-methyl-phenoxy)phenyl]butanal (400 mg, 1.20 mmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (1.03 g, 1.68 mmol, 1.4 equiv.), [2-(2-aminophenyl)phenyl]palladium(1+); bis(1-adamantyl)-butyl-phosphane; methanesulfonate (174.84 mg, 240.08 μmol, 0.2 equiv.), K₂CO₃ (1.5 M, 1.20 mL, 1.5 equiv.) in dioxane (4 mL) and H₂O (1 mL) was degassed and purged with N₂ three times, and then the mixture was stirred at 100° C. for 1 hour at N₂ atmosphere under microwave. The reaction mixture was concentrated under reduced pressure to give a residue. The residue was purified by flash silica gel chromatography (Eluent of 10˜36% ethyl acetate/petroleum ether) to give tert-butyl 6-(8-(benzo[d]thiazol-2-ylcarbamoyl)-3,4-dihydroisoquinolin-2(1H)-yl)-3-(2-methyl-3-(4-(4-oxobutyl)phenoxy)phenyl)picolinate (500 mg, 676.6 μmol, 56.3% yield) as a yellow solid.

MS (ESI) m/z: 739.5 [M+H]⁺

### Step F. Procedure for Preparation of tert-butyl 6-(8-(benzo[d]thiazol-2-ylcarbamoyl)-3,4-dihydroisoquinolin-2(1H)-yl)-3-(3-(4-(4-(4-(3-(2,6-dioxopiperidin-3-yl)-1-methyl-1H-indazol-6-yl)piperazin-1-yl)butyl)phenoxy)-2-methylphenyl)picolinate

A mixture of tert-butyl 6-(8-(benzo[d]thiazol-2-ylcarbamoyl)-3,4-dihydroisoquinolin-2(1H)-yl)-3-(2-methyl-3-(4-(4-oxobutyl)phenoxy)phenyl)picolinate (75 mg, 101.50 μmol, 1 equiv.), 3-(1-methyl-6-piperazin-1-yl-indazol-3-yl)piperidine-2,6-dione (39.88 mg, 121.80 μmol, 1.2 equiv.), 4A MS (10 mg, 101.50 μmol, 1.00 equiv.) and HCOOH (97.53 μg, 2.03 μmol, 0.02 equiv.) in EtOH (2.4 mL) and THE (2.4 mL) was stirred at 25° C. for 2 h. NaBH₃CN (19.14 mg, 304.51 μmol, 3 equiv.) was then added to the mixture, and the ensuing mixture was stirred at 25° C. for 14 h. The mixture was diluted with H₂O (2 mL×2) and extracted with ethyl acetate (2 mL×3). The combined organic layers were filtered and concentrated under reduced pressure to give the crude compound tert-butyl 6-(8-(benzo[d]thiazol-2-ylcarbamoyl)-3,4-dihydroisoquinolin-2(1H)-yl)-3-(3-(4-(4-(4-(3-(2,6-dioxopiperidin-3-yl)-1-methyl-1H-indazol-6-yl)piperazin-1-yl)butyl)phenoxy)-2-methylphenyl)picolinate (100 mg, crude) as a yellow oil, it was used for next step without other purification.

MS (ESI) m/z: 1050.3 [M+H]⁺

### Step G. Procedure for Preparation of 6-(8-(benzo[d]thiazol-2-ylcarbamoyl)-3,4-dihydroisoquinolin-2(1H)-yl)-3-(3-(4-(4-(4-(3-(2,6-dioxopiperidin-3-yl)-1-methyl-1H-indazol-6-yl)piperazin-1-yl)butyl)phenoxy)-2-methylphenyl)picolinic acid

A solution of tert-butyl 6-(8-(benzo[d]thiazol-2-ylcarbamoyl)-3,4-dihydroisoquinolin-2(1H)-yl)-3-(3-(4-(4-(4-(3-(2,6-dioxopiperidin-3-yl)-1-methyl-1H-indazol-6-yl)piperazin-1-yl)butyl)phenoxy)-2-methylphenyl)picolinate (80 mg, 76.17 μmol, 1 equiv.), in TFA (0.8 mL) and DCM (0.8 mL) was stirred at 40° C. for 2 h. The reaction mixture was concentrated under reduced pressure to give a residue. The residue was purified by prep—HPLC to give 6-(8-(benzo[d]thiazol-2-ylcarbamoyl)-3,4-dihydroisoquinolin-2(1H)-yl)-3-(3-(4-(4-(4-(3-(2,6-dioxopiperidin-3-yl)-1-methyl-1H-indazol-6-yl)piperazin-1-yl)butyl)phenoxy)-2-methylphenyl)picolinic acid (27.5 mg, 26.9 μmol, 35.3% yield, 97.0% purity) as a white solid

MS (ESI) m/z: 994.7 [M+H]⁺.

¹H NMR (400 MHz, DMSO-d₆) δ=13.38-12.52 (m, 1H), 10.86 (s, 1H), 8.14 (s, 1H), 8.04 (d, J=7.6 Hz, 1H), 7.80 (d, J=8.0 Hz, 1H), 7.63 (d, J=7.2 Hz, 1H), 7.56-7.50 (m, 2H), 7.50-7.45 (m, 2H), 7.41-7.34 (m, 2H), 7.21-7.16 (m, 3H), 7.01 (d, J=8.8 Hz, 1H), 6.95-6.82 (m, 6H), 5.00 (s, 2H), 4.31-4.23 (m, 1H), 3.95-3.88 (m, 5H), 3.62-3.47 (m, 4H), 3.24-3.15 (m, 4H), 3.03 (t, J=5.6 Hz, 2H), 2.70-2.58 (m, 6H), 2.33-2.26 (m, 1H), 2.21-2.12 (m, 1H), 1.91 (s, 3H), 1.66-1.50 (m, 4H)

### Example 35. Preparation of Compound 164b

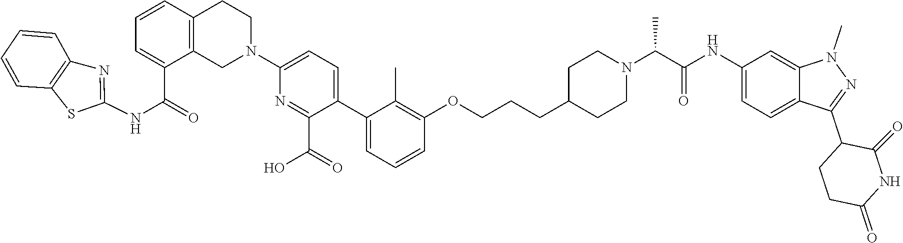 [SMILES: Cc1c(OCCCC2CCN([C@H](C)C(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-(1-((2R)-1-((3-(2,6-dioxopiperidin-3-yl)-1-methyl-1H-indazol-6-yl)amino)-1-oxopropan-2-yl)piperidin-4-yl)propoxy)-2-methylphenyl)picolinic acid

### Step A. Procedure for Preparation of tert-butyl 4-(3-(3-bromo-2-methylphenoxy)propyl)piperidine-1-carboxylate

A mixture of 3-bromo-2-methylphenol (1 g, 5.35 mmol, 1 equiv.), tert-butyl 4-(3-hydroxypropyl)piperidine-1-carboxylate (1.30 g, 5.35 mmol, 1 equiv.) and 2-(tributyl-λ5-phosphanylidene)acetonitrile (1.55 g, 6.42 mmol, 1.2 equiv.) in toluene (20 mL) was degassed and purged with N₂ three times, and then the mixture was stirred at 120° C. for 2 hours under N₂ atmosphere. The reaction mixture was filtered and concentrated under reduced pressure to give a residue. The residue was purified by flash silica gel chromatography (Eluent of 0˜6% ethyl acetate/petroleum ether) to give tert-butyl 4-(3-(3-bromo-2-methylphenoxy)propyl)piperidine-1-carboxylate (2.2 g, 4.8 mmol, 89.8% yield, 90% purity) as a white solid.

¹H NMR (400 MHz, DMSO-d₆) δ=7.18-7.12 (m, 1H), 7.12-7.06 (m, 1H), 6.96 (d, J=8.0 Hz, 1H), 4.02-3.86 (m, 4H), 2.67 (s, 2H), 2.23 (s, 3H), 1.81-1.71 (m, 2H), 1.65 (d, J=12.4 Hz, 2H), 1.49-1.32 (m, 11H), 1.05-0.89 (m, 2H).

### Step B. Procedure for Preparation of 4-(3-(3-bromo-2-methylphenoxy)propyl)piperidine

To a solution of tert-butyl 4-(3-(3-bromo-2-methylphenoxy)propyl)piperidine-1-carboxylate (1.7 g, 3.71 mmol, 90% purity, 1 equiv.) was added HCl/dioxane (4 M, 14.17 mL, 15.27 equiv.). The mixture was stirred at 25° C. for 1 hour. The reaction mixture was filtered and concentrated under reduced pressure to give 4-(3-(3-bromo-2-methylphenoxy)propyl)piperidine (1.5 g, crude, HCl) as a white solid.

¹H NMR (400 MHz, CDCl₃) δ=9.94-9.02 (m, 2H), 7.14 (d, J=8.0 Hz, 1H), 6.99 (t, J=8.0 Hz, 1H), 6.74 (d, J=8.0 Hz, 1H), 3.93 (t, J=6.4 Hz, 2H), 3.50 (d, J=12.4 Hz, 2H), 2.87 (q, J=11.6 Hz, 2H), 2.30 (s, 3H), 1.94 (d, J=13.6 Hz, 2H), 1.86-1.78 (m, 2H), 1.75-1.62 (m, 2H), 1.56-1.49 (m, 2H).

### Step C. Procedure for Preparation of ethyl 2-(4-(3-(3-bromo-2-methylphenoxy)propyl)piperidin-1-yl)propanoate

To a solution of 4-(3-(3-bromo-2-methylphenoxy)propyl)piperidine (1.3 g, 3.73 mmol, 1 eq, HCl) in CH₃CN (25 mL) was added K₂CO₃ (1.55 g, 11.18 mmol, 3 equiv.) and ethyl 2-bromopropanoate (1.01 g, 5.59 mmol, 728.29 μL, 1.5 equiv.). The mixture was stirred at 80° C. for 5 hours. The reaction mixture was filtered and concentrated under reduced pressure to give a residue. The residue was purified by flash silica gel chromatography (Eluent of 0-25% ethyl acetate/petroleum ether) to give ethyl 2-(4-(3-(3-bromo-2-methylphenoxy)propyl)piperidin-1-yl)propanoate (1.2 g, 2.8 mmol, 76.4% yield, 97.8% purity) as a yellow oil.

MS (ESI) m/z: 413.9 [M+H]⁺.

¹H NMR (400 MHz, CDCl₃) δ=7.14 (d, J=8.0 Hz, 1H), 6.99 (t, J=8.0 Hz, 1H), 6.75 (d, J=8.0 Hz, 1H), 4.21-4.15 (m, 2H), 3.93 (t, J=6.4 Hz, 2H), 3.28 (q, J=6.8 Hz, 1H), 2.93 (d, J=10.8 Hz, 2H), 2.34-2.17 (m, 5H), 1.86-1.77 (m, 2H), 1.73 (d, J=9.2 Hz, 2H), 1.46-1.38 (m, 2H), 1.35-1.26 (m, 9H).

### Step D. Procedure for Preparation of (R)-ethyl 2-(4-(3-(3-bromo-2-methylphenoxy)propyl)piperidin-1-yl)propanoate

The ethyl 2-(4-(3-(3-bromo-2-methylphenoxy)propyl)piperidin-1-yl)propanoate residue was further separated by SFC to give (R)-ethyl 2-(4-(3-(3-bromo-2-methylphenoxy)propyl)piperidin-1-yl)propanoate (250 mg, 597.78 μmol, 20.54% yield, 98.6% purity) as a yellow oil and (S)-ethyl 2-(4-(3-(3-bromo-2-methylphenoxy)propyl)piperidin-1-yl)propanoate (250 mg, 555.0 μmol, 19.0% yield, 91.5% purity) as a yellow oil.

¹H NMR (400 MHz, CDCl₃) δ=7.14 (d, J=8.0 Hz, 1H), 6.99 (t, J=8.0 Hz, 1H), 6.75 (d, J=8.4 Hz, 1H), 4.31-4.10 (m, 2H), 3.93 (t, J=6.4 Hz, 2H), 3.33-3.32 (m, 1H), 3.04-2.95 (m, 2H), 2.39-2.15 (m, 5H), 1.87-1.79 (m, 2H), 1.74 (d, J=9.2 Hz, 2H), 1.45-1.39 (m, 2H), 1.37-1.26 (m, 9H).

### Step E. Procedure for Preparation of (R)-tert-butyl 6-(8-(benzo[d]thiazol-2-ylcarbamoyl)-3,4-dihydroisoquinolin-2(1H)-yl)-3-(3-(3-(1-(1-ethoxy-1-oxopropan-2-yl)piperidin-4-yl)propoxy)-2-methylphenyl)picolinate

To a mixture of tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (408.50 mg, 666.89 μmol, 1.1 equiv.), and (R)-ethyl 2-(4-(3-(3-bromo-2-methylphenoxy)propyl)piperidin-1-yl)propanoate (250 mg, 606.27 μmol, 1 equiv.) in dioxane (5 mL) was added Ad₂nBuP Pd G₃(cataCXium® A Pd G₃) (44.15 mg, 60.63 μmol, 0.1 equiv.) and KF (1.5 M, 1.21 mL, 3 equiv.). After addition, the mixture was degassed and purged with N₂ three times, and then the mixture was stirred at 100° C. for 2 hours under N₂ atmosphere. The reaction mixture was filtered and concentrated under reduced pressure to give a residue. The residue was purified by flash silica gel chromatography (Eluent of 0˜60% ethyl acetate/petroleum ether) to give (R)-tert-butyl 6-(8-(benzo[d]thiazol-2-ylcarbamoyl)-3,4-dihydroisoquinolin-2(1H)-yl)-3-(3-(3-(1-(1-ethoxy-1-oxopropan-2-yl)piperidin-4-yl)propoxy)-2-methylphenyl)picolinate (320 mg, 361.1 μmol, 59.5% yield, 92.3% purity) as a yellow solid.

MS (ESI) m/z: 818.8 [M+H]⁺.

### Step F. Procedure for Preparation of (R)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)propanoic acid

To a solution of (R)-tert-butyl 6-(8-(benzo[d]thiazol-2-ylcarbamoyl)-3,4-dihydroisoquinolin-2(1H)-yl)-3-(3-(3-(1-(1-ethoxy-1-oxopropan-2-yl)piperidin-4-yl)propoxy)-2-methylphenyl)picolinate (320 mg, 391.18 μmol, 1 equiv.) in THE (3 mL) was added LiOH·H₂O (49.25 mg, 1.17 mmol, 3 equiv.) and H₂O (0.6 mL). The mixture was stirred at 40° C. for 16 hours. The reaction mixture was concentrated under reduced pressure to remove THF. The aqueous phase was adjusted to pH=4-5 with 1M HCl. The reaction mixture was filtered and diluted in ethyl acetate. The combined organic layers were dried over Na₂SO₄, filtered, and concentrated under reduced pressure to give (R)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)propanoic acid (350 mg, crude) as a yellow solid.

MS (ESI) m/z: 790.7 [M+H]⁺

### Step G. Procedure for Preparation of tert-butyl 6-(8-(benzo[d]thiazol-2-ylcarbamoyl)-3,4-dihydroisoquinolin-2(1H)-yl)-3-(3-(3-(1-((2R)-1-((3-(2,6-dioxopiperidin-3-yl)-1-methyl-11H-indazol-6-yl)amino)-1-oxopropan-2-yl)piperidin-4-yl)propoxy)-2-methylphenyl)picolinate

To a solution of (R)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)propanoic acid (100 mg, 112.93 μmol, 89.21% purity, 1 equiv.), and 3-(6-amino-1-methyl-1H-indazol-3-yl)piperidine-2,6-dione (35.00 mg, 135.52 μmol, 1.2 equiv.) in pyridine (1 mL) was added EDCI (32.47 mg, 169.39 μmol, 1.5 equiv.). The mixture was stirred at 25° C. for 3 hours. The reaction mixture was quenched by addition H₂O (2 mL). The reaction mixture was filtered, washed with 5 mL of water, and diluted in DCM (10 mL). The combined organic layers were dried over Na₂SO₄, filtered, and concentrated under reduced pressure to give tert-butyl 6-(8-(benzo[d]thiazol-2-ylcarbamoyl)-3,4-dihydroisoquinolin-2(1H)-yl)-3-(3-(3-(1-((2R)-1-((3-(2,6-dioxopiperidin-3-yl)-1-methyl-1H-indazol-6-yl)amino)-1-oxopropan-2-yl)piperidin-4-yl)propoxy)-2-methylphenyl)picolinate (100 mg, crude) as a red solid.

MS (ESI) m/z: 1030.8 [M+H]⁺.

### Step H. Procedure for Preparation of 6-(8-(benzo[d]thiazol-2-ylcarbamoyl)-3,4-dihydroisoquinolin-2(1H)-yl)-3-(3-(3-(1-((2R)-1-((3-(2,6-dioxopiperidin-3-yl)-1-methyl-1H-indazol-6-yl)amino)-1-oxopropan-2-yl)piperidin-4-yl)propoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(3-(1-((2R)-1-((3-(2,6-dioxopiperidin-3-yl)-1-methyl-1H-indazol-6-yl)amino)-1-oxopropan-2-yl)piperidin-4-yl)propoxy)-2-methylphenyl)picolinate (100 mg, 97.06 μmol, 1 equiv.) in DCM (1 mL) was added TFA (1.54 g, 13.51 mmol, 1 mL, 139.14 equiv.). The mixture was stirred at 25° C. for 12 hours. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(3-(1-((2R)-1-((3-(2,6-dioxopiperidin-3-yl)-1-methyl-1H-indazol-6-yl)amino)-1-oxopropan-2-yl)piperidin-4-yl)propoxy)-2-methylphenyl)picolinic acid (63.5 mg, 64.6 μmol, 66.5% yield, 99.15% purity) as a white solid.

MS (ESI) m/z: 975.0 [M+H]⁺.

¹H NMR (400 MHz, DMSO-d₆) δ=10.88 (s, 1H), 9.91 (s, 1H), 8.15 (s, 1H), 8.07 (s, 1H), 8.03 (d, J=7.6 Hz, 1H), 7.78 (d, J=8.0 Hz, 1H), 7.62 (d, J=8.8 Hz, 2H), 7.51-7.40 (m, 3H), 7.40-7.30 (m, 2H), 7.19 (dd, J=1.2, 7.6 Hz, 1H), 7.13-7.03 (m, 1H), 6.95 (d, J=8.8 Hz, 1H), 6.87 (d, J=8.4 Hz, 1H), 6.62 (d, J=7.6 Hz, 1H), 4.98 (s, 2H), 4.33-4.29 (m, 1H), 3.98-3.88 (m, 7H), 3.02 (t, J=5.6 Hz, 2H), 2.88-2.77 (m, 2H), 2.68-2.59 (m, 2H), 2.40-2.26 (m, 2H), 2.22-2.10 (m, 2H), 1.89 (s, 3H), 1.82-1.63 (m, 4H), 1.40-1.30 (m, 2H), 1.34-1.09 (m, 7H).

### Example 36. Preparation of Compound 173a

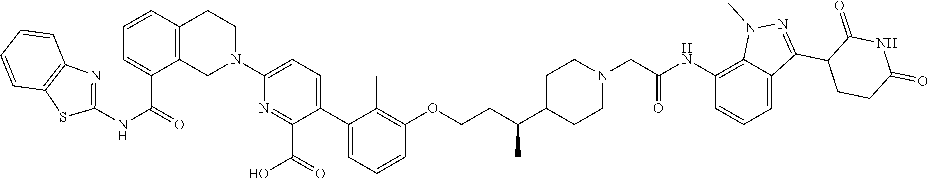 [SMILES: Cc1c(OCC[C@H](C)C2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-((3S)-3-(1-(2-((3-(2,6-dioxopiperidin-3-yl)-1-methyl-1H-indazol-7-yl)amino)-2-oxoethyl)piperidin-4-yl)butoxy)-2-methylphenyl)picolinic acid

### Step A. Procedure for Preparation of (S)-tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)piperidin-4-yl)butoxy)-2-methylphenyl)picolinate

(S)-ethyl 2-(4-(4-(3-bromo-2-methylphenoxy)butan-2-yl)piperidin-1-yl)acetate (90 mg, 218.26 μmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (133.69 mg, 218.26 μmol, 1 equiv.), KF (38.04 mg, 654.77 μmol, 15.34 μL, 3 equiv.) and Ad₂nBuP Pd G₃ (15.89 mg, 21.83 μmol, 0.1 equiv.) were taken up into a microwave tube in dioxane (1 mL) and H₂O (0.1 mL). The sealed tube was heated at 100° C. for 1 hour under microwave. The reaction mixture was concentrated under reduced pressure to give a residue. The residue was purified by prep-TLC (SiO₂, DCM/MeOH=20/1) to give (S)-tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)piperidin-4-yl)butoxy)-2-methylphenyl)picolinate (96 mg, 117.3 μmol, 53.8% yield) as a yellow solid.

MS (ESI) m/z: 818.4 [M+H]⁺.

### Step B. Procedure for Preparation of (S)-2-(4-(4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)butan-2-yl)piperidin-1-yl)acetic acid

To a solution of (S)-tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)piperidin-4-yl)butoxy)-2-methylphenyl)picolinate (90.00 mg, 110.02 μmol, 1 equiv.) in THF (1 mL) and H₂O (0.5 mL) was added LiOH·H₂O (1 M, 330.06 μL, 3 equiv.). The mixture was stirred at 25° C. for 2 hours. The reaction mixture was partitioned between DCM 15 (5 mL×3) mL and H₂O (6 mL). The organic phase was separated, washed with brine 9 mL (3 mL×3), dried over Na₂SO₄, filtered, and concentrated under reduced pressure to give (S)-2-(4-(4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)butan-2-yl)piperidin-1-yl)acetic acid (80.00 mg, crude) as a yellow solid.

MS (ESI) m/z: 818.4 [M+H]⁺.

### Step C. Procedure for Preparation of tert-butyl 6-(8-(benzo[d]thiazol-2-ylcarbamoyl)-3,4-dihydroisoquinolin-2(1H)-yl)-3-(3-((3S)-3-(1-(2-((3-(2,6-dioxopiperidin-3-yl)-1-methyl-11H-indazol-7-yl)amino)-2-oxoethyl)piperidin-4-yl)butoxy)-2-methylphenyl)picolinate

To a solution of (S)-2-(4-(4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)butan-2-yl)piperidin-1-yl)acetic acid (80 mg, 101.27 μmol, 1 equiv.) in pyridine (2 mL) was added dropwise EDCI (19.41 mg, 101.27 μmol, 1 equiv.) at 25° C. After addition, the mixture was stirred at this temperature for 15 minutes, and then 3-(7-amino-1-methyl-1H-indazol-3-yl)piperidine-2,6-dione (31.39 mg, 121.52 μmol, 1.2 equiv.) was added. The resulting mixture was stirred at 40° C. for 12 hours. The reaction mixture was partitioned between DCM (8 mL×3) and H₂O (15 mL). The organic phase was separated, washed with brine (5 mL×3), dried over Na₂SO₄, filtered, and concentrated under reduced pressure to give tert-butyl 6-(8-(benzo[d]thiazol-2-ylcarbamoyl)-3,4-dihydroisoquinolin-2(1H)-yl)-3-(3-((3S)-3-(1-(2-((3-(2,6-dioxopiperidin-3-yl)-1-methyl-1H-indazol-7-yl)amino)-2-oxoethyl)piperidin-4-yl)butoxy)-2-methylphenyl)picolinate (90 mg, 87.4 μmol, 86.3% yield) as a yellow solid.

MS (ESI) m/z: 804.7 [M+H]⁺

### Step D. Procedure for Preparation of 6-(8-(benzo[d]thiazol-2-ylcarbamoyl)-3,4-dihydroisoquinolin-2(1H)-yl)-3-(3-((3S)-3-(1-(2-((3-(2,6-dioxopiperidin-3-yl)-1-methyl-11H-indazol-7-yl)amino)-2-oxoethyl)piperidin-4-yl)butoxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(3-((3S)-3-(1-(2-((3-(2,6-dioxopiperidin-3-yl)-1-methyl-1H-indazol-7-yl)amino)-2-oxoethyl)piperidin-4-yl)butoxy)-2-methylphenyl)picolinate (80 mg, 77.65 μmol, 1 equiv.) in DCM (0.6 mL) and TFA (0.5 mL) was stirred at 25° C. for 12 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-((3S)-3-(1-(2-((3-(2,6-dioxopiperidin-3-yl)-1-methyl-1H-indazol-7-yl)amino)-2-oxoethyl)piperidin-4-yl)butoxy)-2-methylphenyl)picolinic acid (52.8 mg, 53.2 μmol, 68.6% yield, 98.2% purity) as a white solid.

MS (ESI) m/z: 974.5 [M+H]⁺

¹H NMR (400 MHz, DMSO-d₆) δ=12.82 (s, 1H), 12.71-12.39 (m, 1H), 10.98-10.86 (m, 1H), 10.57-10.47 (m, 1H), 8.06-8.00 (m, 1H), 7.79 (d, J=8.0 Hz, 1H), 7.68 (d, J=8.4 Hz, 1H), 7.63 (d, J=7.6 Hz, 1H), 7.50-7.42 (m, 3H), 7.40-7.33 (m, 2H), 7.22-7.18 (m, 1H), 7.16-7.07 (m, 2H), 6.97 (d, J=8.8 Hz, 1H), 6.91 (d, J=8.4 Hz, 1H), 6.66-6.61 (m, 1H), 5.02-4.94 (m, 2H), 4.46-4.35 (m, 1H), 4.31-4.17 (m, 2H), 4.09-4.02 (m, 3H), 3.92 (t, J=6.0 Hz, 2H), 3.77 (s, 1H), 3.63-3.52 (m, 2H), 3.03 (t, J=5.6 Hz, 2H), 2.73-2.61 (m, 2H), 2.33 (d, J=2.0 Hz, 2H), 2.25-2.11 (m, 2H), 1.89 (d, J=2.4 Hz, 3H), 1.85-1.79 (m, 2H), 1.70-1.49 (m, 6H), 1.26-1.20 (m, 1H), 0.96-0.88 (m, 3H)

### Example 37. Preparation of Compound 174b

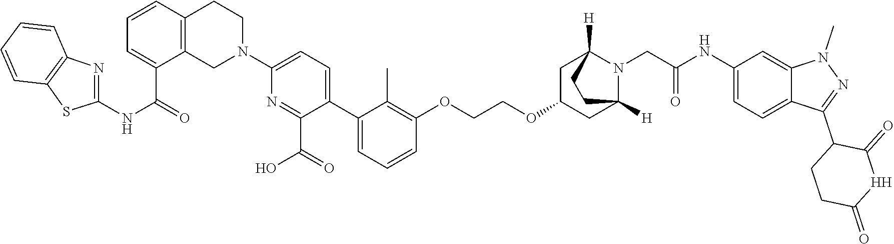

### 6-(8-(benzo[d]thiazol-2-ylcarbamoyl)-3,4-dihydroisoquinolin-2(1H)-yl)-3-(3-(2-(((1R,3s,5S)-8-(2-((3-(2,6-dioxopiperidin-3-yl)-1-methyl-1H-indazol-6-yl)amino)-2-oxoethyl)-8-azabicyclo[3.2.1]octan-3-yl)oxy)ethoxy)-2-methylphenyl)picolinic acid

### Step A. Procedure for Preparation of (1R,3s,5S)-tert-butyl 3-(2-ethoxy-2-oxoethoxy)-8-azabicyclo[3.2.1]octane-8-carboxylate

To a solution of (1R,3s,5S)-tert-butyl 3-hydroxy-8-azabicyclo[3.2.1]octane-8-carboxylate (1 g, 4.40 mmol, 1 equiv.) in DCM (20 mL) was added rhodium(II) acetate dimer (97.23 mg, 219.97 μmol, 0.05 equiv.). Ethyl 2-diazoacetate (652.59 mg, 5.72 mmol, 598.71 μL, 1.3 equiv.) was added at 0° C. The mixture was stirred at 25° C. for 3 hours, and then ethyl 2-diazoacetate (251.00 mg, 2.20 mmol, 230.27 μL, 0.5 equiv.) was added at 0° C. The mixture was stirred at 25° C. for 3 hours. DCM (50 mL) and water (50 mL) were added, and layers were separated. The aqueous phase was extracted with DCM (30 mL×2). The combined organic phase was dried over anhydrous sodium sulfate, filtered, and concentrated under vacuum. The residue was purified by flash silica gel chromatography (Eluent of 0˜22% ethyl acetate/petroleum ether) to give compound (1R,3s,5S)-tert-butyl 3-(2-ethoxy-2-oxoethoxy)-8-azabicyclo[3.2.1]octane-8-carboxylate (830 mg, 2.6 mmol, 60.2% yield) as a yellow oil.

MS (ESI) m/z: 314.0 [M+H]⁺

¹H NMR (400 MHz, DMSO-d₆) δ=4.16-4.00 (m, 6H), 3.90-3.78 (m, 1H), 2.02-1.77 (m, 4H), 1.58 (d, J=5.2 Hz, 2H), 1.46-1.37 (m, 9H), 1.35 (s, 2H), 1.23-1.16 (m, 3H)

### Step B. Procedure for Preparation of (1R,3s,5S)-tert-butyl 3-(2-hydroxyethoxy)-8-azabicyclo[3.2.1]octane-8-carboxylate

To a solution of LiAlH₄ (80.42 mg, 2.12 mmol, 0.8 equiv.) in THE (20 mL) was added (1R,3s,5S)-tert-butyl 3-(2-ethoxy-2-oxoethoxy)-8-azabicyclo[3.2.1]octane-8-carboxylate (830 mg, 2.65 mmol, 1 equiv.) in THE (5 mL) at 0° C. The mixture was stirred at 0° C. for 2 hours. The reaction mixture was quenched by addition water (0.1 mL) and 15% NaOH (0.1 mL) and water (0.3 mL). The combined mixture was dried with Na₂SO₄, filtered, and concentrated under reduced pressure to give compound (1R,3s,5S)-tert-butyl 3-(2-hydroxyethoxy)-8-azabicyclo[3.2.1]octane-8-carboxylate (600 mg, 2.21 mmol, 83.5% yield) as a colorless oil.

MS (ESI) m/z: 294.2 [M+23]⁺

¹H NMR (400 MHz, DMSO-d₆) δ=4.53 (s, 1H), 4.06 (s, 2H), 3.82-3.70 (m, 1H), 3.48-3.37 (m, 4H), 2.03-1.75 (m, 5H), 1.68-1.55 (m, 2H), 1.41 (s, 9H), 1.34 (s, 1H)

### Step C. Procedure for Preparation of (1R,3s,5S)-tert-butyl 3-(2-(3-bromo-2-methylphenoxy)ethoxy)-8-azabicyclo[3.2.1]octane-8-carboxylate

To a solution of (1R,3s,5S)-tert-butyl 3-(2-hydroxyethoxy)-8-azabicyclo[3.2.1]octane-8-carboxylate (500 mg, 1.84 mmol, 1 equiv.) in toluene (10 mL) was added 2-(tributyl-λ5-phosphanylidene)acetonitrile (667.09 mg, 2.76 mmol, 1.5 equiv.) and 3-bromo-2-methylphenol (689.27 mg, 3.69 mmol, 2 equiv.). The mixture was stirred at 120° C. for 12 hours. DCM (50 mL) and water (50 mL) were added and layers were separated. The aqueous phase was extracted with DCM (30 mL×2). The combined organic phase was dried over anhydrous sodium sulfate, filtered and concentrated under vacuum. The residue was purified by flash silica gel chromatography (Eluent of 0˜22% ethyl acetate/petroleum ether) to give compound (1R,3s,5S)-tert-butyl 3-(2-(3-bromo-2-methylphenoxy)ethoxy)-8-azabicyclo[3.2.1]octane-8-carboxylate (620 mg, 1.4 mmol, 76.4% yield) as a yellow oil.

MS (ESI) m/z: 385.8 [M+H]⁺

¹H NMR (400 MHz, DMSO-d₆) δ=7.18-7.14 (m, 1H), 7.09 (t, J=8.0 Hz, 1H), 6.98 (d, J=7.6 Hz, 1H), 4.13-4.00 (m, 5H), 3.91-3.81 (m, 1H), 3.76-3.71 (m, 2H), 2.23 (s, 3H), 2.06-1.71 (m, 5H), 1.62 (d, J=5.2 Hz, 2H), 1.40 (s, 9H)

### Step D. Procedure for Preparation of (1R,3s,5S)-3-(2-(3-bromo-2-methylphenoxy)ethoxy)-8-azabicyclo[3.2.1]octane

To a solution of (1R,3s,5S)-tert-butyl 3-(2-(3-bromo-2-methylphenoxy)ethoxy)-8-azabicyclo[3.2.1]octane-8-carboxylate (320 mg, 726.66 μmol, 1 equiv.) in DCM (4 mL) was added HCl/dioxane (4 M, 1.60 mL, 8.81 equiv.). The mixture was stirred at 25° C. for 1 hour. The reaction mixture was concentrated under reduced pressure to remove solvent to give compound (1R,3S,5S)-3-(2-(3-bromo-2-methylphenoxy)ethoxy)-8-azabicyclo[3.2.1]octane (260 mg, 690.2 μmol, 94.9% yield) as a pink solid.

¹H NMR (400 MHz, DMSO-d₆) δ=9.11-8.71 (m, 2H), 7.19-7.15 (m, 1H), 7.10 (t, J=8.0 Hz, 1H), 6.98 (d, J=8.4 Hz, 1H), 4.08 (t, J=4.4 Hz, 2H), 3.98 (s, 2H), 3.57 (s, 2H), 2.24 (s, 3H), 2.09 (d, J=13.2 Hz, 2H), 1.98-1.81 (m, 4H), 1.72-1.56 (m, 2H)

### Step E. Procedure for Preparation of ethyl 2-((1R,3S,5S)-3-(2-(3-bromo-2-methylphenoxy)ethoxy)-8-azabicyclo[3.2.1]octan-8-yl)acetate

To a solution of (1R,3s,5S)-3-(2-(3-bromo-2-methylphenoxy)ethoxy)-8-azabicyclo[3.2.1]octane (300 mg, 796.36 μmol, 1 equiv., HCl) in CH₃CN (3 mL) was added K₂CO₃ (550.32 mg, 3.98 mmol, 5 equiv.) and ethyl 2-bromoacetate (159.59 mg, 955.63 μmol, 105.69 μL, 1.2 equiv.). The mixture was stirred at 60° C. for 2 hours. DCM (30 mL) and water (30 mL) were added, and layers were separated. The aqueous phase was extracted with DCM (15 mL×2). The combined organic phase was dried over anhydrous sodium sulfate, filtered, and concentrated under vacuum. The residue was purified by column chromatography (SiO₂, petroleum ether/ethyl acetate=10/1 to 0/1) to give compound ethyl 2-((1R,3s,5S)-3-(2-(3-bromo-2-methylphenoxy)ethoxy)-8-azabicyclo[3.2.1]octan-8-yl)acetate (320 mg, 750.5 μmol, 94.2% yield) as a yellow oil.

MS (ESI) m/z: 428.1 [M+H]⁺

### Step F. Procedure for Preparation of tert-butyl 6-(8-(benzo[d]thiazol-2-ylcarbamoyl)-3,4-dihydroisoquinolin-2(1H)-yl)-3-(3-(2-(((1R,3S,5S)-8-(2-ethoxy-2-oxoethyl)-8-azabicyclo[3.2.1]octan-3-yl)oxy)ethoxy)-2-methylphenyl)picolinate

To a solution of ethyl 2-((1R,3s,5S)-3-(2-(3-bromo-2-methylphenoxy)ethoxy)-8-azabicyclo[3.2.1]octan-8-yl)acetate (320 mg, 750.57 μmol, 1 equiv.) in dioxane (4 mL) was added tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (459.76 mg, 750.57 μmol, 1 equiv.), K₃PO₄ (477.96 mg, 2.25 mmol, 3 equiv.), H₂O (0.4 mL), and Ad₂nBup-Pd-G₃ (54.66 mg, 75.06 μmol, 0.1 equiv.). The mixture was stirred at 100° C. for 2 hours. The reaction mixture was concentrated under reduced pressure to remove solvent. The residue was purified by flash silica gel chromatography (Eluent of 0˜76% ethyl acetate/petroleum ether) to give compound tert-butyl 6-(8-(benzo[d]thiazol-2-ylcarbamoyl)-3,4-dihydroisoquinolin-2(1H)-yl)-3-(3-(2-(((1R,3S,5S)-8-(2-ethoxy-2-oxoethyl)-8-azabicyclo[3.2.1]octan-3-yl)oxy)ethoxy)-2-methylphenyl)picolinate (400 mg, 480.8 μmol, 64.0% yield) as a yellow solid.

MS (ESI) m/z: 832.5 [M+H]⁺

### Step G. Procedure for Preparation of 2-((1R,3s,5S)-3-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethoxy)-8-azabicyclo[3.2.1]octan-8-yl)acetic acid

To a solution of tert-butyl 6-(8-(benzo[d]thiazol-2-ylcarbamoyl)-3,4-dihydroisoquinolin-2(1H)-yl)-3-(3-(2-(((1R,3s,5S)-8-(2-ethoxy-2-oxoethyl)-8-azabicyclo[3.2.1]octan-3-yl)oxy)ethoxy)-2-methylphenyl)picolinate (350 mg, 420.67 μmol, 1 equiv.) in THE (4 mL) and H₂O (1 mL) was added LiOH—H₂O (52.96 mg, 1.26 mmol, 3 equiv.). The mixture was stirred at 25° C. for 3 hours. The reaction mixture was concentrated under reduced pressure to remove solvent. To the residue was added 5 mL H₂O. The pH of mixture was adjusted to 4 with 10% citric acid. Then the reaction mixture was filtered and concentrated under reduced pressure to give compound 2-((1R,3sS,5S)-3-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethoxy)-8-azabicyclo[3.2.1]octan-8-yl)acetic acid (320 mg, 398.0 μmol, 94.6% yield) as a yellow solid.

MS (ESI) m/z: 804.3 [M+H]⁺

¹H NMR (400 MHz, DMSO-d₆) δ=13.02-12.73 (m, 1H), 8.01 (d, J=7.6 Hz, 1H), 7.77 (d, J=8.0 Hz, 1H), 7.60 (d, J=7.6 Hz, 1H), 7.47-7.41 (m, 3H), 7.39-7.31 (m, 2H), 7.13-7.06 (m, 1H), 6.97-6.91 (m, 2H), 6.59 (d, J=7.6 Hz, 1H), 5.75 (s, 1H), 4.97 (s, 2H), 3.87 (t, J=5.6 Hz, 2H), 3.17 (s, 2H), 3.03 (t, J=5.6 Hz, 3H), 2.24 (s, 1H), 2.06-1.96 (m, 6H), 1.88 (s, 3H), 1.78-1.68 (m, 6H), 1.01 (s, 9H)

### Step H. Procedure for Preparation of tert-butyl 6-(8-(benzo[d]thiazol-2-ylcarbamoyl)-3,4-dihydroisoquinolin-2(1H)-yl)-3-(3-(2-(((1R,3s,5S)-8-(2-((3-(2,6-dioxopiperidin-3-yl)-1-methyl-1H-indazol-6-yl)amino)-2-oxoethyl)-8-azabicyclo[3.2.1]octan-3-yl)oxy)ethoxy)-2-methylphenyl)picolinate

To a solution of 2-((1R,3s,5S)-3-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethoxy)-8-azabicyclo[3.2.1]octan-8-yl)acetic acid (110 mg, 136.82 μmol, 1 equiv.) in DMF (1 mL) was added HATU (62.43 mg, 164.19 μmol, 1.2 equiv.) and DIEA (53.05 mg, 410.47 μmol, 71.50 μL, 3 equiv.). Then 3-(6-amino-1-methyl-1H-indazol-3-yl)piperidine-2,6-dione (38.87 mg, 150.50 μmol, 1.1 equiv.) was added into the mixture and stirred at 25° C. for 12 hours. DCM (20 mL) and water (20 mL) were added and layers were separated. The aqueous phase was extracted with DCM (10 mL×2). The combined organic phase was dried over anhydrous sodium sulfate, filtered, and concentrated under vacuum to give compound tert-butyl 6-(8-(benzo[d]thiazol-2-ylcarbamoyl)-3,4-dihydroisoquinolin-2(1H)-yl)-3-(3-(2-(((1R,3s,5S)-8-(2-((3-(2,6-dioxopiperidin-3-yl)-1-methyl-1H-indazol-6-yl)amino)-2-oxoethyl)-8-azabicyclo[3.2.1]octan-3-yl)oxy)ethoxy)-2-methylphenyl)picolinate (120 mg, 114.9 μmol, 83.9% yield) as a brown solid.

MS (ESI) m/z: 1044.5 [M+H]⁺

### Step L Procedure for preparation of 6-(8-(benzo[d]thiazol-2-ylcarbamoyl)-3,4-dihydroisoquinolin-2(1H)-yl)-3-(3-(2-(((1R,3s,5S)-8-(2-((3-(2,6-dioxopiperidin-3-yl)-1-methyl-1H-indazol-6-yl)amino)-2-oxoethyl)-8-azabicyclo[3.2.1]octan-3-yl)oxy)ethoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(2-(((1R,3s,5S)-8-(2-((3-(2,6-dioxopiperidin-3-yl)-1-methyl-1H-indazol-6-yl)amino)-2-oxoethyl)-8-azabicyclo[3.2.1]octan-3-yl)oxy)ethoxy)-2-methylphenyl)picolinate (120 mg, 114.92 μmol, 1 equiv.) in DCM (2 mL) was added TFA (3.08 g, 27.01 mmol, 2 mL, 235.06 equiv.). The mixture was stirred at 25° C. for 12 hours. The reaction mixture was concentrated under reduced pressure to remove solvent. The residue was purified by prep-HPLC to give compound 6-(8-(benzo[d]thiazol-2-ylcarbamoyl)-3,4-dihydroisoquinolin-2(1H)-yl)-3-(3-(2-(((1R,3s,5S)-8-(2-((3-(2,6-dioxopiperidin-3-yl)-1-methyl-1H-indazol-6-yl)amino)-2-oxoethyl)-8-azabicyclo[3.2.1]octan-3-yl)oxy)ethoxy)-2-methylphenyl)picolinic acid (35.7 mg, 33.2 μmol, 28.9% yield, 96.1% purity) as an off-white solid.

MS (ESI) m/z: 494.9 [(M+2)/2]⁺

¹H NMR (400 MHz, DMSO-d₆) δ=10.87 (s, 1H), 9.91 (s, 1H), 8.15 (s, 1H), 8.05-8.00 (m, 2H), 7.78 (d, J=8.0 Hz, 1H), 7.65-7.60 (m, 2H), 7.49-7.41 (m, 3H), 7.39-7.31 (m, 2H), 7.26-7.21 (m, 1H), 7.14-7.05 (m, 1H), 6.98-6.87 (m, 2H), 6.65 (d, J=7.6 Hz, 1H), 4.97 (s, 2H), 4.37-4.28 (m, 1H), 4.07 (t, J=4.4 Hz, 2H), 3.94-3.88 (m, 5H), 3.77-3.74 (m, 2H), 3.73-3.65 (m, 2H), 3.17 (s, 2H), 3.02 (t, J=5.6 Hz, 2H), 2.68-2.62 (m, 2H), 2.38-2.29 (m, 2H), 2.20-2.14 (m, 1H), 1.91 (s, 7H), 1.66-1.56 (m, 4H)

### Example 38. Preparation of Compound 178

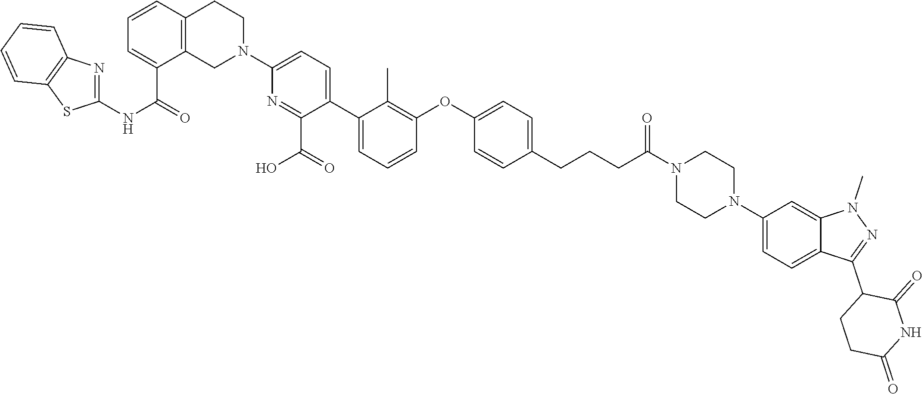 [SMILES: Cc1c(Oc2ccc(CCCC(=O)N3CCN(c4ccc5c(C6CCC(=O)NC6=O)nn(C)c5c4)CC3)cc2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(4-(4-(4-(3-(2,6-dioxopiperidin-3-yl)-1-methyl-1H-indazol-6-yl)piperazin-1-yl)-4-oxobutyl)phenoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(4-(4-ethoxy-4-oxobutyl)phenoxy)-2-methylphenyl)picolinate

Tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (779.34 mg, 1.27 mmol, 1.2 equiv.), ethyl 4-(4-(3-bromo-2-methylphenoxy)phenyl)butanoate (400 mg, 1.06 mmol, 1 equiv.), K₂CO₃ (1.5 M, 1.06 mL, 3 equiv.), and [2-(2-aminophenyl)phenyl]palladium(1+); bis(1-adamantyl)-butylphosphane; methanesulfonate (154.43 mg, 212.05 μmol, 0.2 equiv.) were taken up into a microwave tube in dioxane (4 mL) and H₂O (0.5 mL). The sealed tube was heated at 100° C. for 60 min under microwave. The reaction mixture was concentrated under reduced pressure to give a residue. The residue was purified by flash silica gel chromatography (Eluent of 0˜500% Ethylacetate/petroleum ether) to give tert-butyl 6-(8-(benzo[d]thiazol-2-ylcarbamoyl)-3,4-dihydroisoquinolin-2(1H)-yl)-3-(3-(4-(4-ethoxy-4-oxobutyl)phenoxy)-2-methylphenyl)picolinate (730 mg, crude) as a yellow oil.

MS (ESI) m/z: 783.4 [M+H]⁺.

### Step B. Procedure for Preparation of 4-(4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)phenyl)butanoic acid

A mixture of tert-butyl 6-(8-(benzo[d]thiazol-2-ylcarbamoyl)-3,4-dihydroisoquinolin-2(1H)-yl)-3-(3-(4-(4-ethoxy-4-oxobutyl)phenoxy)-2-methylphenyl)picolinate (243 mg, 310.37 μmol, 1 equiv.), LiOH H₂O (78.14 mg, 1.86 mmol, 5 equiv.) in THF (1.8 mL) and H₂O (0.6 mL) was stirred at 25° C. for 16 hours. The mixture was concentrated to remove the THF. It was then acidified to pH=2˜3 with 1 M HCl (5 mL). A white solid precipitated from the mixture which was collected by filtration to give 4-(4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)phenyl)butanoic acid (212 mg, crude) as a white solid.

MS (ESI) m/z: 755.4 [M+H]⁺.

### Step C. Procedure for Preparation of tert-butyl 6-(8-(benzo[d]thiazol-2-ylcarbamoyl)-3,4-dihydroisoquinolin-2(1H)-yl)-3-(3-(4-(4-(4-(3-(2,6-dioxopiperidin-3-yl)-1-methyl-1H-indazol-6-yl)piperazin-1-yl)-4-oxobutyl)phenoxy)-2-methylphenyl)picolinate

To a solution of 4-(4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)phenyl)butanoic acid (150 mg, 198.70 μmol, 1 equiv.) in DMF (1.5 mL) was added HATU (113.33 mg, 298.06 μmol, 1.5 equiv.) and DIEA (77.04 mg, 596.11 μmol, 103.83 μL, 3 equiv.). The mixture was stirred at 25° C. for 1 hour. Then 3-(1-methyl-6-(piperazin-1-yl)-1H-indazol-3-yl)piperidine-2,6-dione (78.06 mg, 238.45 μmol, 1.2 equiv.) was added. The mixture was stirred at 25° C. for 15 hours. The mixture was diluted with saturated Na₂CO₃ solution (10 mL). The solution was filtered and concentrated under reduced pressure to give tert-butyl 6-(8-(benzo[d]thiazol-2-ylcarbamoyl)-3,4-dihydroisoquinolin-2(1H)-yl)-3-(3-(4-(4-(4-(3-(2,6-dioxopiperidin-3-yl)-1-methyl-1H-indazol-6-yl)piperazin-1-yl)-4-oxobutyl)phenoxy)-2-methylphenyl)picolinate (209 mg crude) as a white solid.

MS (ESI) m/z: 1064.5 [M+H]⁺

### Step D. Procedure for Preparation of 6-(8-(benzo[d]thiazol-2-ylcarbamoyl)-3,4-dihydroisoquinolin-2(1H)-yl)-3-(3-(4-(4-(4-(3-(2,6-dioxopiperidin-3-yl)-1-methyl-1H-indazol-6-yl)piperazin-1-yl)-4-oxobutyl)phenoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(4-(4-(4-(3-(2,6-dioxopiperidin-3-yl)-1-methyl-1H-indazol-6-yl)piperazin-1-yl)-4-oxobutyl)phenoxy)-2-methylphenyl)picolinate (200 mg, 187.92 μmol, 1 equiv.) in DCM (1.5 mL) was added TFA (1.5 mL). The mixture was stirred at 25° C. for 48 hours. The reaction mixture was concentrated under reduced pressure to give a residue. The residue was purified by prep-HPLC to give the compound 6-(8-(benzo[d]thiazol-2-ylcarbamoyl)-3,4-dihydroisoquinolin-2(1H)-yl)-3-(3-(4-(4-(4-(3-(2,6-dioxopiperidin-3-yl)-1-methyl-1H-indazol-6-yl)piperazin-1-yl)-4-oxobutyl)phenoxy)-2-methylphenyl)picolinic acid (26.3 mg, 24.6 μmol, 13.1% yield, 94.4% purity) as a white solid.

MS (ESI) m/z: 1008.5 [M+H]⁺.

¹H NMR (400 MHz, DMSO-d₆) δ=13.02-12.55 (m, 2H), 10.86 (s, 1H), 8.06-8.02 (m, 1H), 7.83-7.76 (m, 1H), 7.67-7.59 (m, 1H), 7.55-7.50 (m, 2H), 7.50-7.43 (m, 2H), 7.40-7.33 (m, 2H), 7.21-7.14 (m, 3H), 7.00 (d, J=8.8 Hz, 1H), 6.94 (d, J=9.2 Hz, 1H), 6.90-6.80 (m, 5H), 4.99 (s, 2H), 4.22 (s, 1H), 3.95-3.88 (m, 5H), 3.61 (d, J=15.6 Hz, 4H), 3.19 (d, J=15.6 Hz, 4H), 3.03 (t, J=5.6 Hz, 2H), 2.62-2.56 (m, 4H), 2.38 (t, J=7.2 Hz, 2H), 2.33-2.27 (m, 1H), 2.19-2.11 (m, 1H), 1.90 (s, 3H), 1.81 (m, J=7.2 Hz, 2H).

### Example 39. Preparation of Compound 180a

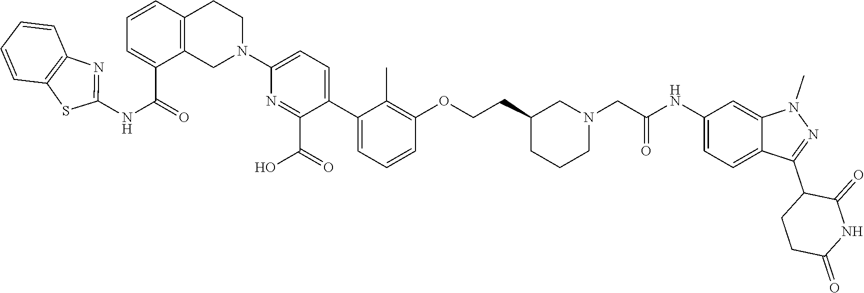 [SMILES: Cc1c(OCC[C@@H]2CCCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(2-((3S)-1-(2-((3-(2,6-dioxopiperidin-3-yl)-1-methyl-1H-indazol-6-yl)amino)-2-oxoethyl)piperidin-3-yl)ethoxy)-2-methylphenyl)picolinic acid

### Step A. Procedure for Preparation of (S)-tert-butyl 3-(2-(3-bromo-2-methylphenoxy)ethyl)piperidine-1-carboxylate

To a solution of 3-bromo-2-methylphenol (815.62 mg, 4.36 mmol, 1 equiv.) in toluene (10 mL) was added 2-(tributyl-λ⁵-phosphanylidene)acetonitrile (1.58 g, 6.54 mmol, 1.5 equiv.) and (S)-tert-butyl 3-(2-hydroxyethyl)piperidine-1-carboxylate (1 g, 4.36 mmol, 1 equiv.). The mixture was stirred at 120° C. for 2 hours under N₂. The reaction mixture was concentrated under reduced pressure to a residue. The residue was purified by flash silica gel chromatography (Eluent of 0-10% ethyl acetate/petroleum ether) to give (S)-tert-butyl 3-(2-(3-bromo-2-methylphenoxy)ethyl)piperidine-1-carboxylate (1.6 g, 4.0 mmol, 92.1% yield) as a yellow oil.

MS (ESI) m/z: 314.0 [M+H]⁺

¹H NMR (400 MHz, DMSO-d₆) δ=7.17-7.13 (m, 1H), 7.12-7.06 (m, 1H), 6.98 (d, J=8.0 Hz, 1H), 4.03 (t, J=6.0 Hz, 2H), 3.91-3.69 (m, 2H), 2.23 (s, 3H), 1.80 (d, J=11.8 Hz, 1H), 1.71-1.53 (m, 5H), 1.47-1.24 (m, 12H)

### Step B. Procedure for Preparation of (S)-3-(2-(3-bromo-2-methylphenoxy)ethyl)piperidine

To a solution of (S)-tert-butyl 3-(2-(3-bromo-2-methylphenoxy)ethyl)piperidine-1-carboxylate (1.6 g, 4.02 mmol, 1 equiv.) in HCl/dioxane (20 mL). The mixture was stirred at 25° C. for 1 hour. The reaction mixture was concentrated under reduced pressure to remove solvent, and then it was filtered and concentrated under reduced pressure to give compound (S)-3-(2-(3-bromo-2-methylphenoxy)ethyl)piperidine (1.03 g, 3.1 mmol, 76.6% yield) as a white solid.

¹H NMR (400 MHz, DMSO-d₆) δ=9.06-8.78 (m, 2H), 7.18-7.14 (m, 1H), 7.12-7.06 (m, 1H), 6.98 (d, J=8.0 Hz, 1H), 4.07-3.98 (m, 2H), 3.29-3.21 (m, 1H), 3.18 (d, J=12.0 Hz, 1H), 2.80-2.68 (m, 1H), 2.60 (t, J=12.0 Hz, 1H), 2.24 (s, 3H), 2.04-1.93 (m, 1H), 1.85-1.60 (m, 5H), 1.29-1.14 (m, 1H)

### Step C. Procedure for Preparation of (S)-ethyl 2-(3-(2-(3-bromo-2-methylphenoxy)ethyl)piperidin-1-yl)acetate

To a solution of (S)-3-(2-(3-bromo-2-methylphenoxy)ethyl)piperidine (500 mg, 1.49 mmol, 1 equiv., HCl) in CH₃CN (5 mL) was added K₂CO₃ (1.03 g, 7.47 mmol, 5 equiv.), KI (24.80 mg, 149.40 μmol, 0.1 equiv.), and ethyl 2-bromoacetate (249.49 mg, 1.49 mmol, 165.23 μL, 1 equiv.). The mixture was stirred at 60° C. for 1 hour. The reaction mixture was concentrated under reduced pressure to remove solvent. The residue was purified by flash silica gel chromatography (Eluent of 0˜100% ethyl acetate/petroleum ether) to give (S)-ethyl 2-(3-(2-(3-bromo-2-methylphenoxy)ethyl)piperidin-1-yl)acetate (360 mg, 936.7 μmol, 62.7% yield) as a yellow oil.

MS (ESI) m/z: 386.0 [M+H]⁺

¹H NMR (400 MHz, DMSO-d₆) δ=7.17-7.13 (m, 1H), 7.11-7.06 (m, 1H), 6.97 (d, J=8.0 Hz, 1H), 4.09-4.03 (m, 2H), 4.03-3.97 (m, 2H), 3.16 (s, 2H), 2.80 (d, J=10.4 Hz, 1H), 2.71 (d, J=10.8 Hz, 1H), 2.22 (s, 3H), 2.18-2.10 (m, 1H), 1.95 (t, J=10.0 Hz, 1H), 1.77-1.69 (m, 2H), 1.67-1.59 (m, 2H), 1.59-1.54 (m, 1H), 1.51-1.37 (m, 1H), 1.16 (t, J=7.2 Hz, 3H), 0.99-0.88 (m, 1H)

### Step D. Procedure for Preparation of (S)-tert-butyl 6-(8-(benzo[d]thiazol-2-ylcarbamoyl)-3,4-dihydroisoquinolin-2(1H)-yl)-3-(3-(2-(1-(2-ethoxy-2-oxoethyl)piperidin-3-yl)ethoxy)-2-methylphenyl)picolinate

To a solution of (S)-ethyl 2-(3-(2-(3-bromo-2-methylphenoxy)ethyl)piperidin-1-yl)acetate (300 mg, 780.63 μmol, 1 equiv.) in dioxane (3 mL) was added tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (573.80 mg, 936.75 μmol, 1.2 equiv.), KF (1.5 M, 1.56 mL, 3 equiv.), and [2-(2-aminophenyl)phenyl]palladium(1+); bis(1-adamantyl)-butyl-phosphane; methanesulfonate (56.85 mg, 78.06 μmol, 0.1 equiv.). The mixture was stirred at 100° C. for 1 hour. The reaction mixture was concentrated under reduced pressure to give a residue. The residue was purified by flash silica gel chromatography (Eluent of 0-40% ethyl acetate/petroleum ether) to give (S)-tert-butyl 6-(8-(benzo[d]thiazol-2-ylcarbamoyl)-3,4-dihydroisoquinolin-2(1H)-yl)-3-(3-(2-(1-(2-ethoxy-2-oxoethyl)piperidin-3-yl)ethoxy)-2-methylphenyl)picolinate (450 mg, 541.1 μmol, 69.3% yield, 95% purity) as a yellow solid.

MS (ESI) m/z: 790.5 [M+H]⁺

### Step E. Procedure for Preparation of (S)-2-(3-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethyl)piperidin-1-yl)acetic acid

To a solution of (S)-tert-butyl 6-(8-(benzo[d]thiazol-2-ylcarbamoyl)-3,4-dihydroisoquinolin-2(1H)-yl)-3-(3-(2-(1-(2-ethoxy-2-oxoethyl)piperidin-3-yl)ethoxy)-2-methylphenyl)picolinate (450 mg, 569.64 μmol, 1 equiv.) in THE (5 mL) and H₂O (1.25 mL) was added LiOH·H₂O (71.71 mg, 1.71 mmol, 3 equiv.). The mixture was stirred at 40° C. for 2 hours. The reaction mixture was concentrated under reduced pressure to remove solvent, the residue was added 5 mL H₂O. The pH of mixture was adjusted to 4 with 1M HCl. Then the reaction mixture was filtered and concentrated under reduced pressure to give (S)-2-(3-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethyl)piperidin-1-yl)acetic acid (430 mg, 564.3 μmol, 99.1% yield) as a yellow solid.

MS (ESI) m/z: 762.5 [M+H]⁺

### Step F. Procedure for Preparation of tert-butyl 6-(8-(benzo[d]thiazol-2-ylcarbamoyl)-3,4-dihydroisoquinolin-2(1H)-yl)-3-(3-(2-((3S)-1-(2-((3-(2,6-dioxopiperidin-3-yl)-1-methyl-11H-indazol-6-yl)amino)-2-oxoethyl)piperidin-3-yl)ethoxy)-2-methylphenyl)picolinate

To a solution of (S)-2-(3-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethyl)piperidin-1-yl)acetic acid (130 mg, 170.62 μmol, 1 equiv.) in DMF (1 mL) was added HATU (84.34 mg, 221.81 μmol, 1.3 equiv.) and DIEA (66.15 mg, 511.86 μmol, 89.16 μL, 3 equiv.). To the mixture was added 3-(6-amino-1-methyl-1H-indazol-3-yl)piperidine-2,6-dione (52.88 mg, 204.74 μmol, 1.2 equiv.) and stirred at 25° C. for 12 hours. The reaction mixture was added into water (5 mL), then filtered and concentrated under reduced pressure to give tert-butyl 6-(8-(benzo[d]thiazol-2-ylcarbamoyl)-3,4-dihydroisoquinolin-2(1H)-yl)-3-(3-(2-((3S)-1-(2-((3-(2,6-dioxopiperidin-3-yl)-1-methyl-1H-indazol-6-yl)amino)-2-oxoethyl)piperidin-3-yl)ethoxy)-2-methylphenyl)picolinate (130 mg, 129.7 μmol, 76.0% yield) as a yellow solid.

MS (ESI) m/z: 1003.7 [M+H]⁺

### Step G. Procedure for Preparation of 6-(8-(benzo[d]thiazol-2-ylcarbamoyl)-3,4-dihydroisoquinolin-2(1H)-yl)-3-(3-(2-((3S)-1-(2-((3-(2,6-dioxopiperidin-3-yl)-1-methyl-1H-indazol-6-yl)amino)-2-oxoethyl)piperidin-3-yl)ethoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(2-((3S)-1-(2-((3-(2,6-dioxopiperidin-3-yl)-1-methyl-1H-indazol-6-yl)amino)-2-oxoethyl)piperidin-3-yl)ethoxy)-2-methylphenyl)picolinate (130 mg, 129.72 μmol, 1 equiv.) in DCM (1.5 mL) was added TFA (14.79 mg, 129.72 μmol, 9.60 μL, 1 equiv.). The mixture was stirred at 25° C. for 12 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(2-((3S)-1-(2-((3-(2,6-dioxopiperidin-3-yl)-1-methyl-1H-indazol-6-yl)amino)-2-oxoethyl)piperidin-3-yl)ethoxy)-2-methylphenyl)picolinic acid (53.6 mg, 53.4 μmol, 41.2% yield, 98.8% purity) as an off-white solid.

MS (ESI) m/z: 946.4 [M+H]⁺

¹H NMR (400 MHz, DMSO-d₆) δ=13.20-12.63 (m, 1H), 10.87 (s, 1H), 9.93-9.83 (m, 1H), 8.15-8.12 (m, 1H), 8.07-7.99 (m, 2H), 7.78 (d, J=8.0 Hz, 1H), 7.67-7.57 (m, 2H), 7.52-7.42 (m, 2H), 7.41-7.32 (m, 3H), 7.17 (d, J=8.4 Hz, 1H), 7.06 (t, J=8.0 Hz, 1H), 6.92 (d, J=8.4 Hz, 1H), 6.86 (d, J=8.4 Hz, 1H), 6.65-6.57 (m, 1H), 4.98 (s, 2H), 4.37-4.26 (m, 1H), 4.06-3.97 (m, 2H), 3.95-3.85 (m, 5H), 3.21-3.09 (m, 3H), 3.04-2.98 (m, 2H), 2.95-2.86 (m, 1H), 2.84-2.75 (m, 1H), 2.68-2.60 (m, 2H), 2.35-2.28 (m, 1H), 2.24-2.11 (m, 2H), 2.07-1.98 (m, 1H), 1.88 (s, 3H), 1.86-1.50 (m, 6H)

### Example 40. Preparation of Compound 180b

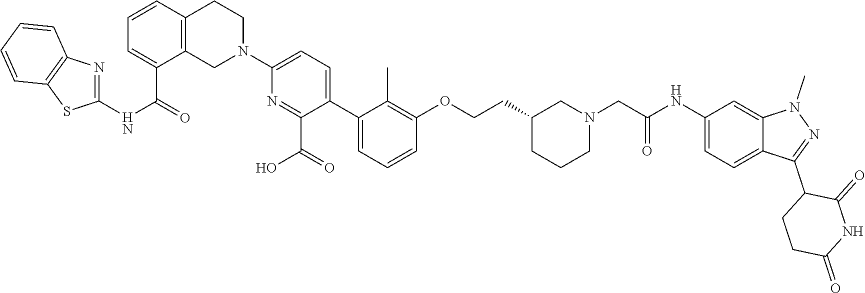 [SMILES: Cc1c(OCC[C@H]2CCCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(2-((3R)-1-(2-((3-(2,6-dioxopiperidin-3-yl)-1-methyl-1H-indazol-6-yl)amino)-2-oxoethyl)piperidin-3-yl)ethoxy)-2-methylphenyl)picolinic acid

### Step A. Procedure for Preparation of (R)-tert-butyl 3-(2-(3-bromo-2-methylphenoxy)ethyl)piperidine-1-carboxylate

A mixture of 3-bromo-2-methylphenol (0.91 g, 4.87 mmol, 1 equiv.), (R)-tert-butyl 3-(2-hydroxyethyl)piperidine-1-carboxylate (1.00 g, 4.38 mmol, 0.9 equiv.), 2-(tributyl-λ⁵-phosphanylidene)acetonitrile (1.76 g, 7.30 mmol, 1.5 equiv.) in toluene (10 mL) was degassed and purged with N₂ three times, and then the mixture was stirred at 120° C. for 2 h under N₂ atmosphere. The reaction mixture was concentrated under reduced pressure to give a residue. The residue was purified by flash silica gel chromatography (Eluent of 0˜ 10% ethyl acetate/petroleum ether) to give (R)-tert-butyl 3-(2-(3-bromo-2-methylphenoxy)ethyl)piperidine-1-carboxylate (1.64 g, crude) as a yellow oil.

¹H NMR (400 MHz, DMSO-d₆) δ=7.15 (d, J=8.0 Hz, 1H), 7.09 (t, J=8.0 Hz, 1H), 6.97 (d, J=8.0 Hz, 1H), 4.03 (t, J=5.6 Hz, 2H), 3.84-3.71 (m, 2H), 2.83-2.64 (m, 2H), 2.23 (s, 3H), 1.81-1.58 (m, 5H), 1.33 (s, 9H), 1.27-1.17 (m, 2H)

### Step B. Procedure for Preparation of (R)-3-(2-(3-bromo-2-methylphenoxy)ethyl)piperidine

A solution of (R)-tert-butyl 3-(2-(3-bromo-2-methylphenoxy)ethyl)piperidine-1-carboxylate (1.64 g, 4.12 mmol, 1 equiv.) in TFA (15 mL) and DCM (15 mL) was stirred at 25° C. for 1 h. The reaction mixture was concentrated under reduced pressure to give a residue. The residue was purified by flash silica gel chromatography (Eluent of 0˜ 10% MeOH/DCM gradient) to give (R)-3-(2-(3-bromo-2-methylphenoxy)ethyl)piperidine (1.34 g, crude) as a yellow oil.

¹H NMR (400 MHz, DMSO-d₆) δ=8.66 (s, 1H), 7.19-7.14 (m, 1H), 7.10 (t, J=8.0 Hz, 1H), 6.98 (d, J=7.6 Hz, 1H), 4.08-4.03 (m, 2H), 3.31-3.20 (m, 2H), 2.81-2.60 (m, 2H), 2.24 (s, 3H), 1.97-1.89 (m, 1H), 1.87-1.53 (m, 6H)

### Step C. Procedure for Preparation of (R)-ethyl 2-(3-(2-(3-bromo-2-methylphenoxy)ethyl)piperidin-1-yl)acetate

A mixture of (R)-3-(2-(3-bromo-2-methylphenoxy)ethyl)piperidine (600 mg, 1.41 mmol, 70% purity, 1 equiv.), ethyl 2-bromoacetate (235.20 mg, 1.41 mmol, 155.76 μL, 1 equiv.), K₂CO₃ (973.22 mg, 7.04 mmol, 5 equiv.), KI (23.38 mg, 140.84 μmol, 0.1 equiv.) in CH₃CN (5 mL) was stirred at 60° C. for 1 h. The reaction mixture was concentrated under reduced pressure to give a residue. The residue was purified by flash silica gel chromatography (Eluent of 0˜100% ethyl acetate/petroleum ether gradient) to give (R)-ethyl 2-(3-(2-(3-bromo-2-methylphenoxy)ethyl)piperidin-1-yl)acetate (420 mg, 1.1 mmol, 77.6% yield, 100% purity) as a white oil.

MS (ESI) m/z: 385.9 [M+H]⁺.

¹H NMR (400 MHz, DMSO-d₆) δ=7.15 (d, J=8.0 Hz, 1H), 7.09 (t, J=8.0 Hz 1H), 6.97 (d, J=8.0 Hz, 1H), 4.09-4.05 (m, 2H), 4.03-3.99 (m, 2H), 3.17 (s, 2H), 2.82-2.70 (m, 1H), 2.73-2.70 (m, 1H), 2.23 (s, 3H), 2.18-1.92 (m, 1H), 1.77-1.57 (m, 5H), 1.50-1.42 (m, 1H), 1.17 (t, J=7.2 Hz, 3H), 0.99-0.92 (m, 1H)

### Step D. Procedure for Preparation of (R)-tert-butyl 6-(8-(benzo[d]thiazol-2-ylcarbamoyl)-3,4-dihydroisoquinolin-2(1H)-yl)-3-(3-(2-(1-(2-ethoxy-2-oxoethyl)piperidin-3-yl)ethoxy)-2-methylphenyl)picolinate

A mixture of tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (621.62 mg, 1.01 mmol, 1.3 equiv.), (R)-ethyl 2-(3-(2-(3-bromo-2-methylphenoxy)ethyl)piperidin-1-yl)acetate (300 mg, 780.63 μmol, 1 equiv.), [2-(2-aminophenyl)phenyl]palladium(1+); bis(1-adamantyl)-butyl-phosphane; methanesulfonate (113.70 mg, 156.13 μmol, 0.2 equiv.), KF (1.5 M, 780.63 μL, 1.5 equiv.) in dioxane (3 mL) was stirred at 100° C. for 1 h under microwave. The reaction mixture was concentrated under reduced pressure to give a residue. The residue was purified by flash silica gel chromatography (Eluent of 0˜ 50% ethyl acetate/petroleum ether) to give (R)-tert-butyl 6-(8-(benzo[d]thiazol-2-ylcarbamoyl)-3,4-dihydroisoquinolin-2(1H)-yl)-3-(3-(2-(1-(2-ethoxy-2-oxoethyl)piperidin-3-yl)ethoxy)-2-methylphenyl)picolinate (573 mg, 725.3 μmol, 92.9% yield) as a yellow oil.

MS (ESI) m/z: 790.4 [M+H]⁺.

### Step E. Procedure for Preparation of (R)-2-(3-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethyl)piperidin-1-yl)acetic acid

A mixture of (R)-tert-butyl 6-(8-(benzo[d]thiazol-2-ylcarbamoyl)-3,4-dihydroisoquinolin-2(1H)-yl)-3-(3-(2-(1-(2-ethoxy-2-oxoethyl)piperidin-3-yl)ethoxy)-2-methylphenyl)picolinate (523 mg, 662.04 μmol, 1 equiv.), LiOH·H₂O (138.91 mg, 3.31 mmol, 5 equiv.) in THE (4 mL) and H₂O (2 mL) was stirred at 40° C. for 1 h. The reaction mixture was diluted with H₂O (2 mL) and concentrated as a turbid liquid. Then the pH of the turbid liquid was adjusted to 3 with 1 N HCl (2 mL). A residue was obtained after trituration and filtered. The residue was used for next step without other purification. The compound (R)-2-(3-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethyl)piperidin-1-yl)acetic acid (458 mg, 601.1 μmol, 90.8% yield) was obtained as a yellow solid.

MS (EIS) m/z: 762.6 [M+H]⁺.

### Step F. Procedure for Preparation of tert-butyl 6-(8-(benzo[d]thiazol-2-ylcarbamoyl)-3,4-dihydroisoquinolin-2(1H)-yl)-3-(3-(2-((3R)-1-(2-((3-(2,6-dioxopiperidin-3-yl)-1-methyl-11H-indazol-6-yl)amino)-2-oxoethyl)piperidin-3-yl)ethoxy)-2-methylphenyl)picolinate

A mixture of (R)-2-(3-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethyl)piperidin-1-yl)acetic acid (80 mg, 105.00 μmol, 1 equiv.), HATU (59.88 mg, 157.50 μmol, 1.5 equiv.), DIPEA (40.71 mg, 314.99 μmol, 54.87 μL, 3 equiv.) in DMF (0.8 mL) was stirred at 25° C. for 0.25 h. Then 3-(6-amino-1-methyl-indazol-3-yl)piperidine-2,6-dione (32.5 mg, 126 μmol, 1.2 equiv.) was added and the mixture was stirred at 40° C. for 2 h. The reaction mixture was quenched by addition brine 5 mL at 25° C., and then diluted and extracted with ethyl acetate (10 mL), concentrated under reduced pressure to give a brown oil. The oil was used for next step without other purification. The compound tert-butyl 6-(8-(benzo[d]thiazol-2-ylcarbamoyl)-3,4-dihydroisoquinolin-2(1H)-yl)-3-(3-(2-((3R)-1-(2-((3-(2,6-dioxopiperidin-3-yl)-1-methyl-1H-indazol-6-yl)amino)-2-oxoethyl)piperidin-3-yl)ethoxy)-2-methylphenyl)picolinate (100 mg, crude) was obtained as a brown oil.

MS (ESI) m/z: 1002.6 [M+H]⁺.

### Step G. Procedure for Preparation of 6-(8-(benzo[d]thiazol-2-ylcarbamoyl)-3,4-dihydroisoquinolin-2(1H)-yl)-3-(3-(2-((3R)-1-(2-((3-(2,6-dioxopiperidin-3-yl)-1-methyl-11H-indazol-6-yl)amino)-2-oxoethyl)piperidin-3-yl)ethoxy)-2-methylphenyl)picolinic acid

A solution of tert-butyl 6-(8-(benzo[d]thiazol-2-ylcarbamoyl)-3,4-dihydroisoquinolin-2(1H)-yl)-3-(3-(2-((3R)-1-(2-((3-(2,6-dioxopiperidin-3-yl)-1-methyl-1H-indazol-6-yl)amino)-2-oxoethyl)piperidin-3-yl)ethoxy)-2-methylphenyl)picolinate (80 mg, 79.83 μmol, 1 equiv.) in DCM (0.5 mL) and TFA (0.5 mL) was stirred at 25° C. for 16 h. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(2-((3R)-1-(2-((3-(2,6-dioxopiperidin-3-yl)-1-methyl-1H-indazol-6-yl)amino)-2-oxoethyl)piperidin-3-yl)ethoxy)-2-methylphenyl)picolinic acid (46.1 mg, 47.2 μmol, 59.1% yield, 96.8% purity) as a white solid.

MS (ESI) m/z: 946.7 [M+H]⁺.

¹H NMR (400 MHz, DMSO-d₆) δ=12.98-12.83 (m, 2H), 10.89 (s, 1H), 10.19-9.64 (m, 1H), 8.04-8.01 (m, 2H), 7.79 (d, J=8.0 Hz, 1H), 7.68-7.63 (m, 2H), 7.50-7.45 (m, 2H), 7.40-7.33 (m, 3H), 7.16-7.07 (m, 2H), 6.97-6.88 (m, 2H), 6.63 (d, J=7.6 Hz, 1H), 4.99 (s, 2H), 4.33 (dd, J=4.4, 9.6 Hz, 1H), 4.05-4.00 (m, 2H), 3.94-3.91 (m, 5H), 3.03 (t, J=5.6 Hz, 2H), 2.68-2.55 (m, 5H), 2.38-2.31 (m, 2H), 2.21-2.13 (m, 2H), 1.91 (s, 3H), 1.86-1.57 (m, 6H), 1.32-0.99 (m, 2H)

### Example 41. Preparation of Compound 181

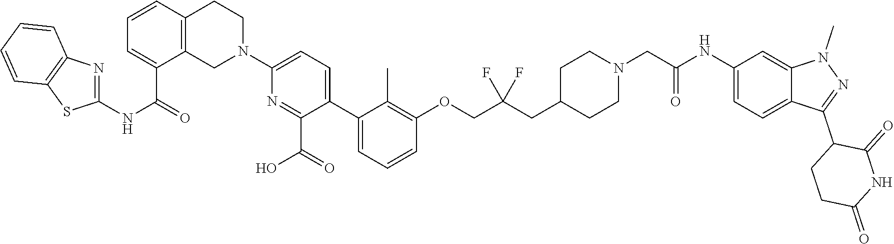 [SMILES: Cc1c(OCC(F)(F)CC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-2-oxo-ethyl]-4-piperidyl]-2,2-difluoro-propoxy]-2-methyl-phenyl]pyridine-2-carboxylic acid

### Step A. Procedure for Preparation of tert-butyl 4-(3-ethoxy-2,2-difluoro-1-hydroxy-3-oxo-propyl)piperidine-1-carboxylate

A mixture of Zn (5.21 g, 79.71 mmol, 2 equiv.) in THE (100 mL) was degassed and purged with N₂ three times and added dropwise 1,2-dibromoethane (748.72 mg, 3.99 mmol, 300.69 μL, 0.1 equiv.) and then the mixture was stirred at 60° C. for 0.5 h under N₂ atmosphere. And then a solution of tert-butyl 4-formylpiperidine-1-carboxylate (8.5 g, 39.86 mmol, 1 equiv.) and ethyl 2-bromo-2,2-difluoro-acetate (11.33 g, 55.80 mmol, 7.17 mL, 1.4 equiv.) in THE (100 mL) was added dropwise to the above solution, and the resulting solution was stirred at 60° C. for 3 hours. The mixture was cooled to 20° C. and then quenched by 1N HCl (200 mL), and then extracted with EtOAc (100 mL×3). The combined organic layers were washed with brine (100 mL×2), dried over Na₂SO₄, filtered and concentrated under reduced pressure to give a residue. The residue was purified by flash silica gel chromatography (Eluent of 0-20% ethyl acetate/petroleum ether) to give compound tert-butyl 4-(3-ethoxy-2,2-difluoro-1-hydroxy-3-oxo-propyl)piperidine-1-carboxylate (19.6 g, crude) as a colorless oil.

¹H NMR (400 MHz, DMSO-d₆) δ=4.35-4.21 (m, 2H), 3.94 (s, 2H), 3.80-3.64 (m, 1H), 3.18-3.16 (m, 1H), 2.67 (s, 2H), 1.82-1.68 (m, 2H), 1.59 (d, J=12.4 Hz, 1H), 1.39 (s, 9H), 1.34-1.12 (m, 5H).

### Step B. Procedure for Preparation of tert-butyl 4-(3-ethoxy-2,2-difluoro-1-methylsulfanylcarbothioyloxy-3-oxo-propyl)piperidine-1-carboxylate

To a solution of tert-butyl 4-(3-ethoxy-2,2-difluoro-1-hydroxy-3-oxo-propyl)piperidine-1-carboxylate (10 g, 29.64 mmol, 1 equiv.) in DMF (100 mL) was added DBU (18.05 g, 118.57 mmol, 17.87 mL, 4 equiv.) and CS₂ (22.57 g, 296.42 mmol, 17.91 mL, 10 equiv.). The mixture was stirred at 20° C. for 1 hour. Then Mel (42.07 g, 296.42 mmol, 18.45 mL, 10 equiv.) was added to the mixture, and the mixture was stirred at 20° C. for 12 hours. The mixture was diluted with water (200 mL) and extracted with EtOAc (100 mL×3). The combined organic layers were washed with brine (100 mL×3), dried over Na₂SO₄, filtered, and concentrated under reduced pressure to give a residue. The residue was purified by flash silica gel chromatography (Eluent of 0˜8% ethyl acetate/petroleum ether) to give compound tert-butyl 4-(3-ethoxy-2,2-difluoro-1-methylsulfanylcarbothioyloxy-3-oxo-propyl)piperidine-1-carboxylate (11 g, 25.7 mmol, 86.8% yield) as a yellow oil.

¹H NMR (400 MHz, CDCl₃) δ=6.40-6.15 (m, 1H), 4.29 (q, J=7.2 Hz, 2H), 4.18-4.10 (m, 2H), 2.80-2.62 (m, 2H), 2.59 (s, 3H), 2.30-2.15 (m, 1H), 1.78 (t, J=11.2 Hz, 2H), 1.45 (s, 10H), 1.33 (t, J=7.2 Hz, 3H).

### Step C. Procedure for Preparation of tert-butyl 4-(3-ethoxy-2,2-difluoro-3-oxo-propyl)piperidine-1-carboxylate

A mixture of tert-butyl 4-(3-ethoxy-2,2-difluoro-1-methylsulfanylcarbothioyloxy-3-oxo-propyl)piperidine-1-carboxylate (10.5 g, 24.56 mmol, 1 equiv.), 2-tert-butylperoxy-2-methyl-propane (3.95 g, 27.02 mmol, 4.98 mL, 1.1 equiv.), phenylphosphonoylbenzene (5.96 g, 29.47 mmol, 1.2 equiv.) in dioxane (100 mL) was degassed and purged with N₂ three times, and then the mixture was stirred at 100° C. for 24 hours under N₂ atmosphere. The reaction mixture was diluted with H₂O (50 mL) and extracted with ethyl acetate (100 mL×2). The combined organic layers were washed with brine 50 mL (25 mL×2), dried over Na₂SO₄, filtered, and concentrated under reduced pressure to give a residue. The residue was purified by flash silica gel chromatography (Eluent of 0˜16% ethyl acetate/petroleum ether) to give compound tert-butyl 4-(3-ethoxy-2,2-difluoro-3-oxo-propyl)piperidine-1-carboxylate (5.1 g, 15.8 mmol, 64.6% yield) as a colorless oil.

¹H NMR (400 MHz, CDCl₃) δ=4.37-4.31 (m, 2H), 4.07 (d, J=12.8 Hz, 2H), 2.71 (t, J=12.8 Hz, 2H), 2.12-1.93 (m, 2H), 1.81-1.71 (m, 3H), 1.46 (s, 9H), 1.37 (t, J=7.2 Hz, 3H), 1.28-1.15 (m, 2H).

### Step D. Procedure for Preparation of tert-butyl 4-(2,2-difluoro-3-hydroxy-propyl)piperidine-1-carboxylate

A mixture of tert-butyl 4-(3-ethoxy-2,2-difluoro-3-oxo-propyl)piperidine-1-carboxylate (5.1 g, 15.87 mmol, 1 equiv.) in THE (60 mL) was added LiAlH₄ (602.27 mg, 15.87 mmol, 1 equiv.), and then the mixture was stirred at 0° C. for 1 hour under N₂ atmosphere. The reaction mixture was quenched by addition H₂O (0.7 mL) at 0° C. The aqueous phase was acidified to pH=4-5 with 1M HCl. The reaction mixture was filtered. The filter cake was diluted in ethyl acetate. The combined organic layers were dried over Na₂SO₄, filtered, and concentrated under reduced pressure to give a residue. The residue was purified by flash silica gel chromatography (Eluent of 0˜18% ethyl acetate/petroleum ether) to give compound tert-butyl 4-(2,2-difluoro-3-hydroxy-propyl)piperidine-1-carboxylate (3.5 g, 11.3 mmol, 71.1% yield, 90% purity) as a colorless oil.

¹H NMR (400 MHz, DMSO-d₆) δ=5.47 (t, J=6.0 Hz, 1H), 3.89 (d, J=12.4 Hz, 2H), 3.61-3.49 (m, 2H), 2.71 (s, 2H), 1.90-1.73 (m, 3H), 1.68 (d, J=13.6 Hz, 2H), 1.38 (s, 9H), 1.14-1.00 (m, 2H).

### Step E. Procedure for Preparation of tert-butyl 4-[3-(3-bromo-2-methyl-phenoxy)-2,2-difluoro-propyl]piperidine-1-carboxylate

A mixture of tert-butyl 4-(2,2-difluoro-3-hydroxy-propyl)piperidine-1-carboxylate (500 mg, 1.79 mmol, 1 equiv.), 1,3-dibromo-2-methyl-benzene (894.77 mg, 3.58 mmol, 2 equiv.), 2,2,6,6-tetramethylheptane-3,5-dione (82.47 mg, 447.51 μmol, 92.14 μL, 0.25 equiv.), CuI (170.46 mg, 895.02 μmol, 0.5 equiv.) and Cs₂CO₃ (699.88 mg, 2.15 mmol, 1.2 equiv.) in NMIP (5 mL) was degassed and purged with N₂ three times, and then the mixture was stirred at 120° C. for 12 hours under N₂ atmosphere. The reaction mixture was quenched by addition water 50 mL, and then extracted with EtOAc (50 mL×3). The combined organic layers were washed with brine (50 mL×3), dried over anhydrous sulfate sodium, filtered, and concentrated under reduced pressure to give a residue. The residue was purified by column chromatography (SiO₂, petroleum ether/ethyl acetate1/0 to 4/1) to give compound tert-butyl 4-[3-(3-bromo-2-methyl-phenoxy)-2,2-difluoro-propyl]piperidine-1-carboxylate (1.78 g, crude) as a yellow oil.

MS (ESI) m/z: 394.2 [M-56+H]⁺

¹H NMR (400 MHz, DMSO-d₆) δ=7.24 (d, J=8.0 Hz, 1H), 7.13 (t, J=8.4 Hz, 1H), 7.05 (d, J=8.0 Hz, 1H), 4.40-4.25 (m, 2H), 3.89 (d, J=12.0 Hz, 2H), 2.83-2.61 (m, 2H), 1.99 (s, 3H), 1.90-1.78 (m, 1H), 1.76-1.64 (m, 2H), 1.38 (s, 9H), 1.16-1.01 (m, 4H).

### Step F. Procedure for Preparation of 4-[3-(3-bromo-2-methyl-phenoxy)-2,2-difluoro-propyl]piperidine

A solution of tert-butyl 4-[3-(3-bromo-2-methyl-phenoxy)-2,2-difluoro-propyl]piperidine-1-carboxylate (1.78 g, 3.97 mmol, 1 equiv.) in HCl/EtOAc (4 M, 15 mL, 15.11 equiv.) was stirred at 25° C. for 2 hours. The reaction mixture was concentrated under reduced pressure to give 4-[3-(3-bromo-2-methyl-phenoxy)-2,2-difluoro-propyl]piperidine (2 g, crude) as a yellow solid.

MS (ESI) m/z: 348.2 [M+H]⁺

### Step G. Procedure for Preparation of ethyl 2-[4-[3-(3-bromo-2-methyl-phenoxy)-2,2-difluoro-propyl]-1-piperidyl]acetate

To a solution of 4-[3-(3-bromo-2-methyl-phenoxy)-2,2-difluoro-propyl]piperidine (500 mg, 1.44 mmol, 1 equiv.) and ethyl 2-bromoacetate (239.79 mg, 1.44 mmol, 158.80 μL, 1 equiv.) in CH₃CN (5 mL) was added K₂CO₃ (595.33 mg, 4.31 mmol, 3 equiv.). The mixture was stirred at 60° C. for 1 hour. The reaction mixture was filtered and concentrated under reduced pressure to give a residue. The residue was purified by column chromatography (SiO₂, petroleum ether/ethyl acetate1/0 to 1/1) to give compound ethyl 2-[4-[3-(3-bromo-2-methyl-phenoxy)-2,2-difluoro-propyl]-1-piperidyl]acetate (380 mg, 874.9 μmol, 60.9% yield) as a yellow oil.

MS (ESI) m/z: 436.0 [M+H]⁺

¹H NMR (400 MHz, DMSO-d₆) δ=7.23 (d, J=8.0 Hz, 1H), 7.12 (t, J=8.0 Hz, 1H), 7.05 (d, J=7.6 Hz, 1H), 4.38-4.25 (m, 2H), 4.06 (q, J=7.2 Hz, 2H), 3.15 (s, 2H), 2.78 (d, J=10.8 Hz, 2H), 2.25 (s, 2H), 2.14 (t, J=11.2 Hz, 2H), 2.06-1.91 (m, 2H), 1.72-1.63 (m, 2H), 1.34-1.21 (m, 2H), 1.21-1.14 (m, 5H).

### Step H. Procedure for Preparation of tert-butyl 6-[8-(1,3-benzothiazol-2-ylcarbamoyl)-3,4-dihydro-1H-isoquinolin-2-yl]-3-[3-[3-[1-(2-ethoxy-2-oxo-ethyl)-4-piperidyl]-2,2-difluoro-propoxy]-2-methyl-phenyl]pyridine-2-carboxylate

Ethyl 2-[4-[3-(3-bromo-2-methyl-phenoxy)-2,2-difluoro-propyl]-1-piperidyl]acetate (380 mg, 874.94 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (535.94 mg, 874.94 μmol, 1 equiv.), [2-(2-aminophenyl)phenyl]palladium(1+); bis(1-adamantyl)-butyl-phosphane; methanesulfonate (63.71 mg, 874.94 μmol, 0.1 equiv.), and KF (1.5 M, 1.75 mL, 3 equiv.) were taken up into a microwave tube in dioxane (2 mL). The sealed tube was heated at 100° C. for 60 minutes under microwave. The reaction mixture was concentrated under reduced pressure to give a residue. The residue was purified by column chromatography (SiO₂, petroleum ether/ethyl acetate10/1 to 1/1) to give compound tert-butyl 6-[8-(1,3-benzothiazol-2-ylcarbamoyl)-3,4-dihydro-1H-isoquinolin-2-yl]-3-[3-[3-[1-(2-ethoxy-2-oxo-ethyl)-4-piperidyl]-2,2-difluoro-propoxy]-2-methyl-phenyl]pyridine-2-carboxylate (512 mg, 609.5 μmol, 69.6% yield) as a yellow solid.

MS (ESI) m/z: 840.5 [M+H]⁺

¹H NMR (400 MHz, DMSO-d₆) δ=12.92-12.81 (m, 1H), 8.02 (d, J=8.4 Hz, 1H), 7.77 (d, J=8.4 Hz, 1H), 7.60 (d, J=7.6 Hz, 1H), 7.45 (d, J=8.8 Hz, 3H), 7.40-7.31 (m, 3H), 7.14 (t, J=8.0 Hz, 1H), 7.00 (d, J=8.4 Hz, 1H), 6.95 (d, J=8.4 Hz, 1H), 6.65 (d, J=7.6 Hz, 1H), 4.98 (s, 2H), 4.35-4.21 (m, 2H), 4.06-4.03 (m, 2H), 3.87 (t, J=5.6 Hz, 2H), 3.14 (s, 2H), 3.03 (t, J=5.6 Hz, 2H), 2.76 (d, J=11.2 Hz, 2H), 2.12 (t, J=10.8 Hz, 2H), 1.99 (s, 3H), 1.89 (s, 3H), 1.71-1.58 (m, 3H), 1.17 (d, J=2.0 Hz, 3H), 1.00 (s, 9H).

### Step I. Procedure for Preparation of 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-2,2-difluoro-propyl]-1-piperidyl]acetic acid

To a solution of tert-butyl 6-[8-(1,3-benzothiazol-2-ylcarbamoyl)-3,4-dihydro-1H-isoquinolin-2-yl]-3-[3-[3-[1-(2-ethoxy-2-oxo-ethyl)-4-piperidyl]-2,2-difluoro-propoxy]-2-methyl-phenyl]pyridine-2-carboxylate (512 mg, 609.53 μmol, 1 equiv.) in H₂O (2.5 mL) and THE (2.5 mL) was added LiOH (72.99 mg, 3.05 mmol, 5 equiv.). The mixture was stirred at 25° C. for 2 hours. The reaction mixture was quenched by addition HCl (lM, 10 mL) and then extracted with CH₂Cl₂ (20 mL×3). The combined organic layers were dried over anhydrous sodium sulfate, filtered, and concentrated under reduced pressure to give compound 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-2,2-difluoro-propyl]-1-piperidyl]acetic acid (600 mg, crude) as a yellow solid.

Step J. Procedure for Preparation of tert-butyl 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-2-oxo-ethyl]-4-piperidyl]-2,2-difluoro-propoxy]-2-methyl-phenyl]pyridine-2-carboxylate

To a solution of 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-2,2-difluoro-propyl]-1-piperidyl]acetic acid (60 mg, 73.90 μmol, 1 equiv.) and 3-(6-amino-1-methyl-indazol-3-yl)piperidine-2,6-dione (20.99 mg, 81.29 μmol, 1.1 equiv.) in DMF (1 mL) was added DIEA (28.65 mg, 221.69 μmol, 38.61 μL, 3 equiv.) and HATU (42.15 mg, 110.85 μmol, 1.5 equiv.). The mixture was stirred at 25° C. for 1 hour. The reaction mixture was quenched by addition water 10 mL, filtered, and concentrated under reduced pressure to give compound tert-butyl 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-2-oxo-ethyl]-4-piperidyl]-2,2-difluoro-propoxy]-2-methyl-phenyl]pyridine-2-carboxylate (120 mg, crude) as a yellow solid.

MS (ESI) m/z: 1052.5 [M+H]⁺

### Step K. Procedure for Preparation of 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-2-oxo-ethyl]-4-piperidyl]-2,2-difluoro-propoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-2-oxo-ethyl]-4-piperidyl]-2,2-difluoro-propoxy]-2-methyl-phenyl]pyridine-2-carboxylate (120 mg, 114.05 μmol, 1 equiv.) in DCM (1 mL) was added TFA (13.00 mg, 114.05 μmol, 8.44 μL, 1 equiv.). The mixture was stirred at 25° C. for 12 hours. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-2-oxo-ethyl]-4-piperidyl]-2,2-difluoro-propoxy]-2-methyl-phenyl]pyridine-2-carboxylic acid (35.7 mg, 34.8 μmol, 30.5% yield, 97.1% purity) as a white solid.

MS (ESI) m/z: 996.8 [M+H]⁺

¹H NMR (400 MHz, DMSO-d₆) δ=10.88 (s, 1H), 9.82 (s, 1H), 8.06-8.00 (m, 2H), 7.78 (d, J=8.0 Hz, 1H), 7.62 (d, J=8.8 Hz, 2H), 7.49-7.42 (m, 3H), 7.40-7.31 (m, 2H), 7.24-7.18 (m, 1H), 7.12 (t, J=8.0 Hz, 1H), 7.00-6.92 (m, 2H), 6.71 (d, J=7.6 Hz, 1H), 4.98 (s, 2H), 4.35-4.24 (m, 3H), 3.95-3.88 (m, 5H), 3.12 (s, 2H), 3.03 (t, J=5.6 Hz, 2H), 2.86 (d, J=11.2 Hz, 2H), 2.68-2.59 (m, 2H), 2.39-2.28 (m, 2H), 2.23-2.14 (m, 3H), 2.11-1.97 (m, 2H), 1.93 (s, 3H), 1.78-1.71 (m, 2H), 1.49-1.35 (m, 2H).

### Example 42. Preparation of Compound 187a

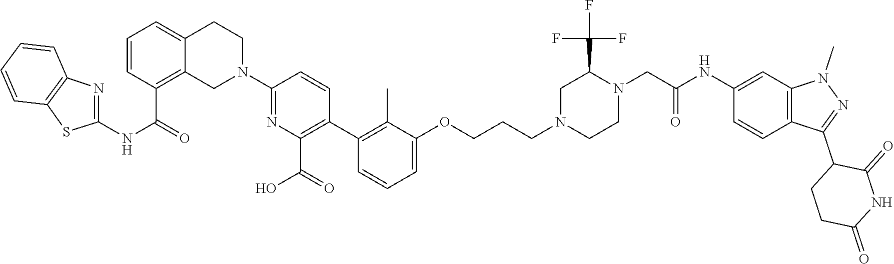 [SMILES: Cc1c(OCCCN2CCN(CC(=O)Nc3ccc4c(C5CCC(=O)NC5=O)nn(C)c4c3)[C@H](C(F)(F)F)C2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-((3S)-4-(2-((3-(2,6-dioxopiperidin-3-yl)-1-methyl-1H-indazol-6-yl)amino)-2-oxoethyl)-3-(trifluoromethyl)piperazin-1-yl)propoxy)-2-methylphenyl)picolinic acid

### Step A. Procedure for Preparation of (S)-tert-butyl 4-(2-ethoxy-2-oxoethyl)-3-(trifluoromethyl)piperazine-1-carboxylate

A mixture of (S)-tert-butyl 3-(trifluoromethyl) piperazine-1-carboxylate (300 mg, 1.18 mmol, 1 equiv.), ethyl 2-bromoacetate (1.18 g, 7.08 mmol, 782.99 μL, 6 equiv.) and DIEA (762.48 mg, 5.90 mmol, 1.03 mL, 5 equiv.) in THE (3 mL) was degassed and purged with N₂ three times, and then the mixture was stirred at 60° C. for 3 hours under N₂ atmosphere. The reaction mixture was diluted with water (20 mL) and extracted with EtOAc (20 mL×3). The combined organic layers were washed with brine (20 mL×3), dried over anhydrous sodium sulfate, filtered, and concentrated under reduced pressure to give a residue. The residue was purified by flash silica gel chromatography (Eluent of 0˜20% ethyl acetate/petroleum ether) to give (S)-tert-butyl 4-(2-ethoxy-2-oxoethyl)-3-(trifluoromethyl)piperazine-1-carboxylate (140 mg, 411.36 μmol, 34.86% yield) as a colorless oil.

¹H NMR (400 MHz, CD₃Cl) δ=4.20-4.11 (m, 2H), 4.01-3.79 (m, 1H), 3.76-3.61 (m, 2H), 3.60-3.50 (m, 1H), 3.49-3.40 (m, 2H), 3.39-3.24 (m, 1H), 3.04-2.77 (m, 2H), 1.43 (s, 9H), 1.25 (t, J=7.2 Hz, 3H).

### Step B. Procedure for Preparation of (S)-ethyl 2-(2-(trifluoromethyl)piperazin-1-yl) acetate

A mixture of (S)-tert-butyl 4-(2-ethoxy-2-oxoethyl)-3-(trifluoromethyl)piperazine-1-carboxylate (140 mg, 411.36 μmol, 1 equiv.) in HCl/dioxane (2 mL) was degassed and purged with N₂ three times, and then the mixture was stirred at 25° C. for 4 hours under N₂ atmosphere. The reaction mixture was concentrated under reduced pressure to give (S)-ethyl 2-(2-(trifluoromethyl)piperazin-1-yl) acetate (150 mg, crude) as a yellow solid.

### Step C. Procedure for Preparation of 1-bromo-3-(3-bromopropoxy)-2-methylbenzene

A mixture of 1,3-dibromopropane (16.19 g, 80.20 mmol, 8.18 mL, 5 equiv.), 3-bromo-2-methylphenol (3 g, 16.04 mmol, 1 equiv.), and K₂CO₃ (6.65 g, 48.12 mmol, 3 equiv.) in CH₃CN (30 mL) was degassed and purged with N₂ three times, and then the mixture was stirred at 70° C. for 16 hours under N₂ atmosphere. The reaction mixture was concentrated under reduced pressure to give a residue. The residue was purified by flash silica gel chromatography (Eluent of 0˜5% ethyl acetate/petroleum ether) to give compound 1-bromo-3-(3-bromopropoxy)-2-methylbenzene (3.6 g, 11.69 mmol, 72.87% yield) as a colorless oil.

¹H NMR (400 MHz, DMSO-d₆) δ=7.20-7.15 (m, 1H), 7.10 (t, J=8.0 Hz, 1H), 6.99 (d, J=8.0 Hz, 1H), 4.09 (t, J=6.0 Hz, 2H), 3.69 (t, J=6.4 Hz, 2H), 3.61 (t, J=6.4 Hz, 1H), 2.30-2.26 (m, 2H), 2.24 (s, 3H).

### Step D. Procedure for Preparation of (S)-ethyl 2-(4-(3-(3-bromo-2-methylphenoxy)propyl)-2-(trifluoromethyl)piperazin-1-yl)acetate

A mixture of (S)-ethyl 2-(2-(trifluoromethyl)piperazin-1-yl)acetate (150 mg, 542.14 μmol, 1 equiv., HCl), 1-bromo-3-(3-bromopropoxy)-2-methylbenzene (200.38 mg, 650.56 μmol, 1.2 equiv.), and K₂CO₃ (449.56 mg, 3.25 mmol, 6 equiv.) in CH₃CN (4 mL) was degassed and purged with N₂ three times, and then the mixture was stirred at 60° C. for 16 hours under N₂ atmosphere. The reaction mixture was concentrated under reduced pressure to give a residue. The residue was purified by flash silica gel chromatography (Eluent of 0-30% ethyl acetate/petroleum ether) to give compound (S)-ethyl 2-(4-(3-(3-bromo-2-methylphenoxy)propyl)-2-(trifluoromethyl)piperazin-1-yl)acetate (100 mg, 205.1 μmol, 37.5% yield, 95.8% purity) as a colorless oil.

MS (ESI) m/z: 469.0 [M+H]⁺

### Step E. Procedure for Preparation of (S)-tert-butyl 6-(8-(benzo[d]thiazol-2-ylcarbamoyl)-3,4-dihydroisoquinolin-2(1H)-yl)-3-(3-(3-(4-(2-ethoxy-2-oxoethyl)-3-(trifluoromethyl)piperazin-1-yl)propoxy)-2-methylphenyl)picolinate

(S)-ethyl 2-(4-(3-(3-bromo-2-methylphenoxy)propyl)-2-(trifluoromethyl)piperazin-1-yl)acetate (100 mg, 213.99 μmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (157.29 mg, 256.78 μmol, 1.2 equiv.), Ad₂nBuP Pd G₃(cataCXium® A Pd G₃) (15.58 mg, 21.40 μmol, 0.1 equiv.), and KF (1.5 M, 427.97 uL, 3 equiv.) were taken up into a microwave tube in dioxane (2.5 mL). The sealed tube was heated at 100° C. for 1 hour under microwave. The reaction mixture was diluted with water (20 mL) and extracted with EtOAc (25 mL×3). The combined organic layers were washed with brine (15 mL×3), dried over anhydrous sodium sulfate, filtered, and concentrated under reduced pressure to give a residue. The residue was purified by prep-TLC (SiO₂, petroleum ether:ethyl acetate=2:1) to give (S)-tert-butyl 6-(8-(benzo[d]thiazol-2-ylcarbamoyl)-3,4-dihydroisoquinolin-2(1H)-yl)-3-(3-(3-(4-(2-ethoxy-2-oxoethyl)-3-(trifluoromethyl)piperazin-1-yl)propoxy)-2-methylphenyl)picolinate (150 mg, 171.82 μmol, 80.30% yield) as a yellow oil.

MS (ESI) m/z: 873.3 [M+H]⁺

### Step F. Procedure for Preparation of (S)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-2-(trifluoromethyl)piperazin-1-yl)acetic acid

A mixture of (S)-tert-butyl 6-(8-(benzo[d]thiazol-2-ylcarbamoyl)-3,4-dihydroisoquinolin-2(1H)-yl)-3-(3-(3-(4-(2-ethoxy-2-oxoethyl)-3-(trifluoromethyl)piperazin-1-yl)propoxy)-2-methylphenyl)picolinate (155 mg, 177.55 μmol, 1 equiv.) and LiOH·H₂O (22.35 mg, 532.65 μmol, 3 equiv.) in THE (2 mL) and H₂O (0.5 mL) was degassed and purged with N₂ three times, and then the mixture was stirred at 25° C. for 16 hours under N₂ atmosphere. The reaction mixture was diluted with water (20 mL) and extracted with EtOAc (25 mL×3). The combined organic layers were washed with brine (15 mL×3), dried over anhydrous sodium sulfate, filtered and concentrated under reduced pressure to give compound (S)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-2-(trifluoromethyl)piperazin-1-yl)acetic acid (150 mg, crude) as a yellow solid.

MS (ESI) m/z: 845.3 [M+H]⁺

¹H NMR (400 MHz, DMSO-d₆) δ=13.06-12.60 (m, 1H), 10.73-10.30 (m, 1H), 8.02 (d, J=8.0 Hz, 1H), 7.78 (d, J=8.0 Hz, 1H), 7.60 (d, J=7.6 Hz, 1H), 7.49-7.41 (m, 3H), 7.40-7.31 (m, 2H), 7.12 (t, J=8.0 Hz, 1H), 6.98-6.90 (m, 2H), 6.61 (d, J=7.6 Hz, 1H), 4.98 (d, J=4.0 Hz, 2H), 4.38-4.13 (m, 1H), 3.87 (t, J=5.6 Hz, 2H), 3.64-3.47 (m, 4H), 3.09-2.97 (m, 4H), 2.28-2.10 (m, 2H), 1.99 (s, 3H), 1.90 (s, 2H), 1.17 (t, J=7.2 Hz, 4H), 1.02 (s, 9H).

### Step G. Procedure for Preparation of tert-butyl 6-(8-(benzo[d]thiazol-2-ylcarbamoyl)-3,4-dihydroisoquinolin-2(1H)-yl)-3-(3-(3-((3S)-4-(2-((3-(2,6-dioxopiperidin-3-yl)-1-methyl-11H-indazol-6-yl)amino)-2-oxoethyl)-3-(trifluoromethyl)piperazin-1-yl)propoxy)-2-methylphenyl)picolinate

A mixture of (S)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-2-(trifluoromethyl)piperazin-1-yl)acetic acid (75 mg, 88.76 μmol, 1 equiv.), 3-(6-amino-1-methyl-indazol-3-yl)piperidine-2,6-dione (25.22 mg, 97.64 μmol, 1.1 equiv.), and DIEA (34.42 mg, 266.29 μmol, 46.38 μL, 3 equiv.) in DMF (1 mL) was degassed and purged with N₂ three times. The mixture was then stirred at 25° C. for 5 minutes. After 5 minutes, HATU (40.50 mg, 106.52 μmol, 1.2 equiv.) was added, and the mixture was stirred at 25° C. for 1 hour under N₂ atmosphere. The resulting reaction mixture was diluted with water (20 mL) and extracted with EtOAc (20 mL×3). The combined organic layers were washed with brine (20 mL×6), dried over anhydrous sodium sulfate, filtered, and concentrated under reduced pressure to give tert-butyl 6-(8-(benzo[d]thiazol-2-ylcarbamoyl)-3,4-dihydroisoquinolin-2(1H)-yl)-3-(3-(3-((3S)-4-(2-((3-(2,6-dioxopiperidin-3-yl)-1-methyl-1H-indazol-6-yl)amino)-2-oxoethyl)-3-(trifluoromethyl)piperazin-1-yl)propoxy)-2-methylphenyl)picolinate (100 mg, crude) as a brown solid.

MS (ESI) m/z: 1085.4 [M+H]⁺

### Step H. Procedure for Preparation of 6-(8-(benzo[d]thiazol-2-ylcarbamoyl)-3,4-dihydroisoquinolin-2(1H)-yl)-3-(3-(3-((3S)-4-(2-((3-(2,6-dioxopiperidin-3-yl)-1-methyl-11H-indazol-6-yl)amino)-2-oxoethyl)-3-(trifluoromethyl)piperazin-1-yl)propoxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(3-(3-((3S)-4-(2-((3-(2,6-dioxopiperidin-3-yl)-1-methyl-1H-indazol-6-yl)amino)-2-oxoethyl)-3-(trifluoromethyl)piperazin-1-yl)propoxy)-2-methylphenyl)picolinate (100 mg, 92.15 μmol, 1 equiv.) in TFA (1 mL) and DCM (1 mL) as degassed and purged with N₂ three times, and then the mixture was stirred at 25° C. for 1 hour under N₂ atmosphere. The reaction mixture was concentrated under reduced pressure to give a residue. The residue was purified by prep-HPLC to give compound 6-(8-(benzo[d]thiazol-2-ylcarbamoyl)-3,4-dihydroisoquinolin-2(1H)-yl)-3-(3-(3-((3S)-4-(2-((3-(2,6-dioxopiperidin-3-yl)-1-methyl-1H-indazol-6-yl)amino)-2-oxoethyl)-3-(trifluoromethyl)piperazin-1-yl)propoxy)-2-methylphenyl)picolinic acid (32.1 mg, 30.7 μmol, 33.4% yield, 98.4% purity) as an off-white solid.

MS (ESI) m/z: 1029.4 [M+H]⁺

¹H NMR (400 MHz, DMSO-d₆) δ=13.03-12.32 (m, 2H), 10.90 (s, 1H), 9.79 (s, 1H), 8.14 (s, 1H), 8.03 (d, J=8.0 Hz, 1H), 7.79 (d, J=8.0 Hz, 1H), 7.65-7.57 (m, 2H), 7.50-7.41 (m, 3H), 7.40-7.32 (m, 2H), 7.25-7.19 (m, 1H), 7.13-7.04 (m, 2H), 6.96 (d, J=8.8 Hz, 1H), 6.86 (d, J=8.0 Hz, 1H), 6.63 (d, J=7.6 Hz, 1H), 4.98 (s, 2H), 4.41-4.34 (m, 1H), 4.08 (s, 3H), 4.01 (t, J=5.6 Hz, 2H), 3.91 (t, J=5.6 Hz, 2H), 3.79-3.67 (m, 2H), 3.48 (d, J=16.8 Hz, 1H), 3.06-2.97 (t, J=5.2 Hz, 3H), 2.87-2.75 (m, 2H), 2.75-2.62 (m, 2H), 2.61-2.53 (m, 3H), 2.48-2.41 (m, 2H), 2.40-2.31 (m, 1H), 2.21-2.12 (m, 1H), 1.96-1.85 (m, 5H).

### Example 43. Preparation of Compound 187b

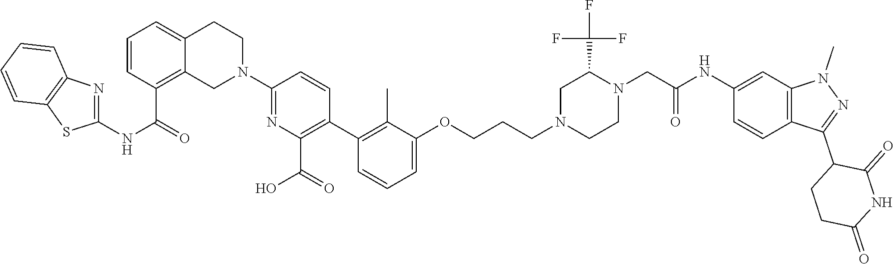 [SMILES: Cc1c(OCCCN2CCN(CC(=O)Nc3ccc4c(C5CCC(=O)NC5=O)nn(C)c4c3)[C@@H](C(F)(F)F)C2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-((3R)-4-(2-((3-(2,6-dioxopiperidin-3-yl)-1-methyl-1H-indazol-6-yl)amino)-2-oxoethyl)-3-(trifluoromethyl)piperazin-1-yl)propoxy)-2-methylphenyl)picolinic acid

### Step A. Procedure for Preparation of (R)-tert-butyl 4-(2-ethoxy-2-oxoethyl)-3-(trifluoromethyl)piperazine-1-carboxylate

To a solution of (R)-tert-butyl 3-(trifluoromethyl)piperazine-1-carboxylate (700 mg, 2.75 mmol, 1 equiv.) in THE (5 mL) was added DIEA (711.65 mg, 5.51 mmol, 959.10 μL, 2.0 equiv.) and ethyl 2-bromoacetate (597.73 mg, 3.58 mmol, 395.84 μL, 1.3 equiv.). The mixture was stirred at 25° C. for 12 hours. The reaction mixture was concentrated under reduced pressure to remove solvent to give a residue. The residue was purified by column chromatography (SiO₂, petroleum ether/ethyl acetate=10/1 to 4/1) to give a compound (R)-tert-butyl 4-(2-ethoxy-2-oxoethyl)-3-(trifluoromethyl)piperazine-1-carboxylate (300 mg, 537.7 μmol, 45.7% yield, 61% purity) as a white oil.

MS (ESI) m/z: 240.5 [M-100+1]⁺

¹H NMR (400 MHz, DMSO-d₆) δ=4.14-4.01 (m, 2H), 3.70-3.52 (m, 3H), 3.32 (s, 3H), 1.38 (s, 9H), 1.23-1.11 (m, 4H), 0.94 (d, J=6.4 Hz, 2H)

### Step B. Procedure for Preparation of (R)-ethyl 2-(2-(trifluoromethyl)piperazin-1-yl)acetate

To a solution of (R)-tert-butyl 4-(2-ethoxy-2-oxoethyl)-3-(trifluoromethyl)piperazine-1-carboxylate (250 mg, 734.56 μmol, 1 equiv.) was added HCl/dioxane (4 M, 183.64 μL, 1 equiv.). The mixture was stirred at 25° C. for 2 hours. The reaction mixture was concentrated under reduced pressure to remove solvent and provide (R)-ethyl 2-(2-(trifluoromethyl)piperazin-1-yl)acetate (240 mg, crude, HCl) as a white solid.

### Step C. Procedure for Preparation of (R)-ethyl 2-(4-(3-(3-bromo-2-methylphenoxy)propyl)-2-(trifluoromethyl)piperazin-1-yl)acetate

To a solution of 1-bromo-3-(3-bromopropoxy)-2-methylbenzene (307.72 mg, 999.08 μmol, 1.2 equiv.) in CH₃CN (10 mL) was added K₂CO₃ (345.20 mg, 2.50 mmol, 3 equiv.) and (R)-ethyl 2-(2-(trifluoromethyl)piperazin-1-yl)acetate (200 mg, 832.56 μmol, 1 equiv.). The mixture was stirred at 60° C. for 12 hours. The reaction mixture was concentrated under reduced pressure to remove solvent. The resulting residue was purified by column chromatography (SiO₂, petroleum ether/ethyl acetate=10/1 to 4/1) to give (R)-ethyl 2-(4-(3-(3-bromo-2-methylphenoxy)propyl)-2-(trifluoromethyl)piperazin-1-yl)acetate (200 mg, 425.53 μmol, 51.11% yield, 99.43% purity) as a white oil.

MS (ESI) m/z: 469.3 [M+H]⁺

### Step D. Procedure for Preparation of (R)-tert-butyl 6-(8-(benzo[d]thiazol-2-ylcarbamoyl)-3,4-dihydroisoquinolin-2(1H)-yl)-3-(3-(3-(4-(2-ethoxy-2-oxoethyl)-3-(trifluoromethyl)piperazin-1-yl)propoxy)-2-methylphenyl)picolinate

To a solution of (R)-ethyl 2-(4-(3-(3-bromo-2-methylphenoxy)propyl)-2-(trifluoromethyl)piperazin-1-yl)acetate (180 mg, 385.18 μmol, 1 equiv.) in dioxane (5 mL) was added K₂CO₃ (1.5 M, 385.18 μL, 1.5 equiv.), [2-(2-aminophenyl)phenyl]palladium(1+); bis(1-adamantyl)-butyl-phosphane; methanesulfonate (56.10 mg, 77.04 μmol, 0.2 equiv.), and tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (283.13 mg, 462.22 μmol, 1.2 equiv.). The mixture was stirred at 100° C. for 1 hour. The reaction mixture was concentrated under reduced pressure to remove solvent. The residue was purified by column chromatography (SiO₂, petroleum ether/ethyl acetate=10/1 to 2/1) to give (R)-tert-butyl 6-(8-(benzo[d]thiazol-2-ylcarbamoyl)-3,4-dihydroisoquinolin-2(1H)-yl)-3-(3-(3-(4-(2-ethoxy-2-oxoethyl)-3-(trifluoromethyl)piperazin-1-yl)propoxy)-2-methylphenyl)picolinate (160 mg, 174.1 μmol, 45.2% yield, 95% purity) as a colorless oil.

MS (ESI) m/z: 873.7 [M+H]⁺

### Step E. Procedure for Preparation of (R)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-2-(trifluoromethyl)piperazin-1-yl)acetic acid

To a solution of (R)-tert-butyl 6-(8-(benzo[d]thiazol-2-ylcarbamoyl)-3,4-dihydroisoquinolin-2(1H)-yl)-3-(3-(3-(4-(2-ethoxy-2-oxoethyl)-3-(trifluoromethyl)piperazin-1-yl)propoxy)-2-methylphenyl)picolinate (150 mg, 171.82 μmol, 1 equiv.) in THE (4 mL) was added LiOH (12.34 mg, 515.47 μmol, 3 equiv.) and H₂O (1 mL). The mixture was stirred at 25° C. for 2 hours. The pH was adjusted to 6 with 1M HCl. The residue was diluted with H₂O (10 mL) and extracted with DCM (10 mL×3). The combined organic layers were washed with aqueous brine (10 mL×3), dried over Na₂SO₄, filtered and concentrated under reduced pressure to give a compound (R)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-2-(trifluoromethyl)piperazin-1-yl)acetic acid (130 mg, crude) was obtained as a yellow solid.

MS (ESI) m/z: 845.5 [M+H]⁺

### Step F. Procedure for Preparation of tert-butyl 6-(8-(benzo[d]thiazol-2-ylcarbamoyl)-3,4-dihydroisoquinolin-2(1H)-yl)-3-(3-(3-((3R)-4-(2-((3-(2,6-dioxopiperidin-3-yl)-1-methyl-11H-indazol-6-yl)amino)-2-oxoethyl)-3-(trifluoromethyl)piperazin-1-yl)propoxy)-2-methylphenyl)picolinate

To a solution of (R)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-2-(trifluoromethyl)piperazin-1-yl)acetic acid (65 mg, 76.93 μmol, 1 equiv.) in DMF (2 mL) was added HATU (35.10 mg, 92.31 μmol, 1.2 equiv.), 3-(6-amino-1-methyl-1H-indazol-3-yl)piperidine-2,6-dione (23.84 mg, 92.31 μmol, 1.2 equiv.), and DIEA (29.83 mg, 230.79 μmol, 40.20 μL, 3 equiv.). The mixture was stirred at 25° C. for 1 hour. The residue was diluted with H₂O (5 mL) and extracted with DCM (5 mL×3). The combined organic layers were washed with aqueous brine (5 mL×3), dried over Na₂SO₄, filtered, and concentrated under reduced pressure to give a compound tert-butyl 6-(8-(benzo[d]thiazol-2-ylcarbamoyl)-3,4-dihydroisoquinolin-2(1H)-yl)-3-(3-(3-((3R)-4-(2-((3-(2,6-dioxopiperidin-3-yl)-1-methyl-1H-indazol-6-yl)amino)-2-oxoethyl)-3-(trifluoromethyl)piperazin-1-yl)propoxy)-2-methylphenyl)picolinate (100 mg, crude) as a brown oil.

MS (ESI) m/z: 543.1 [(M+H)/2]⁺

### Step G. Procedure for Preparation of 6-(8-(benzo[d]thiazol-2-ylcarbamoyl)-3,4-dihydroisoquinolin-2(1H)-yl)-3-(3-(3-((3R)-4-(2-((3-(2,6-dioxopiperidin-3-yl)-1-methyl-11H-indazol-6-yl)amino)-2-oxoethyl)-3-(trifluoromethyl)piperazin-1-yl)propoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(3-((3R)-4-(2-((3-(2,6-dioxopiperidin-3-yl)-1-methyl-1H-indazol-6-yl)amino)-2-oxoethyl)-3-(trifluoromethyl)piperazin-1-yl)propoxy)-2-methylphenyl)picolinate (100 mg, 92.15 μmol, 1 equiv.) in DCM (1 mL) was added TFA (10.51 mg, 92.15 μmol, 6.82 μL, 1 equiv.). The mixture was stirred at 25° C. for 1 hour. The reaction mixture was concentrated under reduced pressure to remove solvent. The residue was purified by prep-HPLC to give a compound 6-(8-(benzo[d]thiazol-2-ylcarbamoyl)-3,4-dihydroisoquinolin-2(1H)-yl)-3-(3-(3-((3R)-4-(2-((3-(2,6-dioxopiperidin-3-yl)-1-methyl-1H-indazol-6-yl)amino)-2-oxoethyl)-3-(trifluoromethyl)piperazin-1-yl)propoxy)-2-methylphenyl)picolinic acid (37.52 mg, 35.6 μmol, 38.8% yield, 97.7% purity) as a yellow gum.

MS (ESI) m/z: 1029.8 [M+H]⁺

¹H NMR (400 MHz, DMSO-d₆) δ=12.87 (d, J=2.0 Hz, 1H), 12.73-12.39 (m, 1H), 10.88 (s, 1H), 9.90 (s, 1H), 8.10-8.00 (m, 2H), 7.79 (d, J=7.6 Hz, 1H), 7.67-7.59 (m, 2H), 7.49-7.31 (m, 5H), 7.16-7.06 (m, 2H), 6.98 (d, J=8.8 Hz, 1H), 6.88 (d, J=8.0 Hz, 1H), 6.64 (d, J=7.6 Hz, 1H), 4.98 (s, 2H), 4.35-4.28 (m, 1H), 4.02 (t, J=5.6 Hz, 2H), 3.96-3.89 (m, 5H), 3.76-3.61 (m, 2H), 3.49 (m, 2H), 3.08-2.88 (m, 4H), 2.86-2.71 (m, 2H), 2.69-2.56 (m, 4H), 2.34-2.31 (m, 2H), 2.20-2.13 (m, 1H), 1.96-1.85 (m, 5H)

### Example 44. Preparation of Compound 190

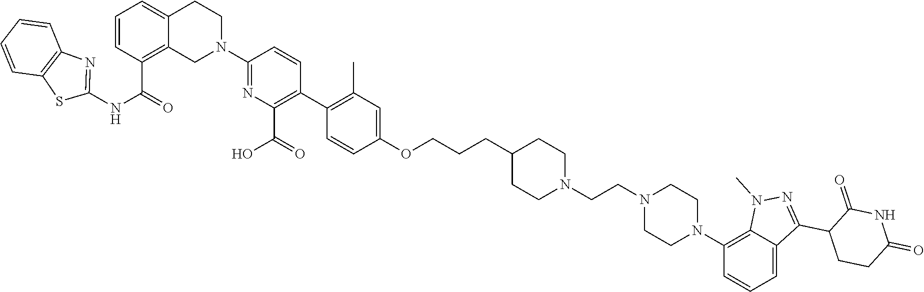 [SMILES: Cc1cc(OCCCC2CCN(CCN3CCN(c4cccc5c(C6CCC(=O)NC6=O)nn(C)c45)CC3)CC2)ccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(4-(3-(1-(2-(4-(3-(2,6-dioxopiperidin-3-yl)-1-methyl-1H-indazol-7-yl)piperazin-1-yl)ethyl)piperidin-4-yl)propoxy)-2-methylphenyl)picolinic acid

### Step A. Procedure for Preparation of tert-butyl 4-(3-(4-bromo-3-methylphenoxy)propyl)piperidine-1-carboxylate

To a solution of tert-butyl 4-(3-bromopropyl)piperidine-1-carboxylate (2.0 g, 6.53 mmol, 1.0 equiv.) in CH₃CN (20 mL) was added K₂CO₃ (2.71 g, 19.59 mmol, 3.0 equiv.) and 4-bromo-3-methyl-phenol (1.83 g, 9.80 mmol, 1.5 equiv.). The mixture was stirred at 60° C. for 16 hours. The reaction mixture was concentrated under reduced pressure to give tert-butyl 4-(3-(4-bromo-3-methylphenoxy)propyl)piperidine-1-carboxylate (2.5 g, crude) as a yellow solid.

MS (ESI) m/z: 313.8 [M-100+H]⁺.

### Step B. Procedure for Preparation of 4-(3-(4-bromo-3-methylphenoxy)propyl)piperidine

To a solution of tert-butyl 4-(3-(4-bromo-3-methylphenoxy)propyl)piperidine-1-carboxylate (2.5 g, 6.06 mmol, 1.0 equiv.) in HCl/EtOAc (15 mL). The mixture was stirred at 25° C. for 1 hour. The mixture was filtered and concentrated under reduced pressure to give compound 4-(3-(4-bromo-3-methylphenoxy)propyl)piperidine (1.2 g, 3.4 mmol, 56.7% yield) as an off-white solid.

### Step C. Procedure for Preparation of 4-[3-(4-bromo-3-methyl-phenoxy)propyl]-1-(2,2-diethoxyethyl)piperidine

To a solution of 4-(3-(4-bromo-3-methylphenoxy)propyl)piperidine (500 mg, 1.43 mmol, 1.0 equiv., HCl) in CH₃CN (5 mL) was added K₂CO₃ (594.51 mg, 4.30 mmol, 3.0 equiv.) and 2-bromo-1,1-diethoxyethane (282.57 mg, 1.43 mmol, 215.70 μL, 1.0 equiv.). The mixture was stirred at 80° C. for 5 hours. The reaction mixture was concentrated under reduced pressure to remove solvent. The residue was purified by flash silica gel chromatography (Eluent of 0-5% DCM/MeOH) to give 4-[3-(4-bromo-3-methyl-phenoxy)propyl]-1-(2,2-diethoxyethyl)piperidine (400 mg, 924.3 μmol, 64.4% yield, 99% purity) as a white solid.

MS (ESI) m/z: 430.0 [M+H]⁺.

¹H NMR (400 MHz, DMSO-d₆) δ=7.42 (d, J=8.8 Hz, 1H), 6.94 (d, J=2.8 Hz, 1H), 6.73-6.68 (m, 1H), 4.55 (t, J=5.2 Hz, 1H), 3.91 (t, J=6.4 Hz, 2H), 3.62-3.52 (m, 2H), 3.50-3.39 (m, 3H), 2.85 (d, J=11.2 Hz, 2H), 2.36 (d, J=5.2 Hz, 2H), 2.29 (s, 3H), 1.95 (t, J=11.2 Hz, 2H), 1.73-1.64 (m, 2H), 1.60 (d, J=11.6 Hz, 2H), 1.36-1.25 (m, 2H), 1.10 (t, J=7.2 Hz, 8H)

### Step D. Procedure for Preparation of tert-butyl 6-(8-(benzo[d]thiazol-2-ylcarbamoyl)-3,4-dihydroisoquinolin-2(1H)-yl)-3-(4-(3-(1-(2,2-diethoxyethyl)piperidin-4-yl)propoxy)-2-methylphenyl)picolinate

A mixture of 4-[3-(4-bromo-3-methyl-phenoxy)propyl]-1-(2,2-diethoxyethyl)piperidine (300 mg, 700.28 μmol, 1.0 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (514.74 mg, 840.33 μmol, 1.2 equiv.), and KF (1.5 M, 1.40 mL, 3.0 equiv.) in dioxane (3 mL) was degassed and purged with N₂ three times, and then [2-(2-aminophenyl)phenyl]palladium(1+); bis(1-adamantyl)-butyl-phosphane; methanesulfonate (51.00 mg, 70.03 μmol, 0.1 equiv.) was added into the mixture. The resulting mixture was stirred at 100° C. for 1 hour under N₂ atmosphere. The reaction mixture was concentrated under reduced pressure to remove solvent. The residue was purified by flash silica gel chromatography (Eluent of 0˜100% ethyl acetate/petroleum ether) to give tert-butyl 6-(8-(benzo[d]thiazol-2-ylcarbamoyl)-3,4-dihydroisoquinolin-2(1H)-yl)-3-(4-(3-(1-(2,2-diethoxyethyl)piperidin-4-yl)propoxy)-2-methylphenyl)picolinate (400 mg, 479.5 μmol, 68.4% yield) as a yellow oil.

MS (ESI) m/z: 834.2 [M+H]⁺.

### Step E. Procedure for Preparation of 6-(8-(benzo[d]thiazol-2-ylcarbamoyl)-3,4-dihydroisoquinolin-2(1H)-yl)-3-(2-methyl-4-(3-(1-(2-oxoethyl)piperidin-4-yl)propoxy)phenyl)picolinic acid

A solution of tert-butyl 6-(8-(benzo[d]thiazol-2-ylcarbamoyl)-3,4-dihydroisoquinolin-2(1H)-yl)-3-(4-(3-(1-(2,2-diethoxyethyl)piperidin-4-yl)propoxy)-2-methylphenyl)picolinate (200 mg, 239.79 μmol, 1 equiv.) in HCOOH (1.5 mL) was stirred at 105° C. for 1 hour. The reaction mixture was concentrated under reduced pressure to give 6-(8-(benzo[d]thiazol-2-ylcarbamoyl)-3,4-dihydroisoquinolin-2(1H)-yl)-3-(2-methyl-4-(3-(1-(2-oxoethyl)piperidin-4-yl)propoxy)phenyl)picolinic acid (150 mg, crude) as a yellow solid.

MS (ESI) m/z: 704.4 [M+H]⁺

### Step F. Procedure for Preparation of 6-(8-(benzo[d]thiazol-2-ylcarbamoyl)-3,4-dihydroisoquinolin-2(1H)-yl)-3-(4-(3-(1-(2-(4-(3-(2,6-dioxopiperidin-3-yl)-1-methyl-1H-indazol-7-yl)piperazin-1-yl)ethyl)piperidin-4-yl)propoxy)-2-methylphenyl)picolinic acid

To a solution of 3-(1-methyl-6-(piperazin-1-yl)-1H-indazol-3-yl)piperidine-2,6-dione (93.05 mg, 255.74 μmol, 1.2 equiv., HCl) in DCM (3 mL) was added NaBH(OAc)₃ (135.50 mg, 639.34 μmol, 3.0 equiv.) and 6-(8-(benzo[d]thiazol-2-ylcarbamoyl)-3,4-dihydroisoquinolin-2(1H)-yl)-3-(2-methyl-4-(3-(1-(2-oxoethyl)piperidin-4-yl)propoxy)phenyl)picolinic acid (150 mg, 213.11 μmol, 1.0 equiv.) at 0° C. The mixture was stirred at 0° C. for 1 hour. Then the mixture was stirred at 25° C. for 11 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(4-(3-(1-(2-(4-(3-(2,6-dioxopiperidin-3-yl)-1-methyl-1H-indazol-7-yl)piperazin-1-yl)ethyl)piperidin-4-yl)propoxy)-2-methylphenyl)picolinic acid (22.6 mg, 21.8 μmol, 10.2% yield, 97.7% purity) as a white solid.

MS (ESI) m/z: 1015.7 [M+H]⁺

¹H NMR (400 MHz, DMSO-d₆) δ=10.88 (s, 1H), 8.01 (d, J=8.0 Hz, 1H), 7.77 (d, J=8.0 Hz, 1H), 7.61 (d, J=7.2 Hz, 1H), 7.48-7.30 (m, 6H), 7.01 (d, J=4.4 Hz, 2H), 6.94 (d, J=8.4 Hz, 1H), 6.88 (d, J=9.2 Hz, 1H), 6.77 (s, 1H), 6.71-6.66 (m, 1H), 4.95 (s, 2H), 4.36-4.30 (m, 1H), 4.23 (s, 3H), 3.94 (s, 2H), 3.90-3.86 (m, 2H), 2.99 (s, 6H), 2.93-2.77 (m, 4H), 2.70-2.53 (m, 8H), 2.36-2.26 (m, 2H), 2.17-2.10 (m, 2H), 2.04 (s, 3H), 1.69 (d, J=6.8 Hz, 2H), 1.66-1.56 (m, 2H), 1.30 (s, 2H), 1.25-1.20 (m, 1H), 1.18-1.06 (m, 2H)

### Example 45. Preparation of Compound 193a

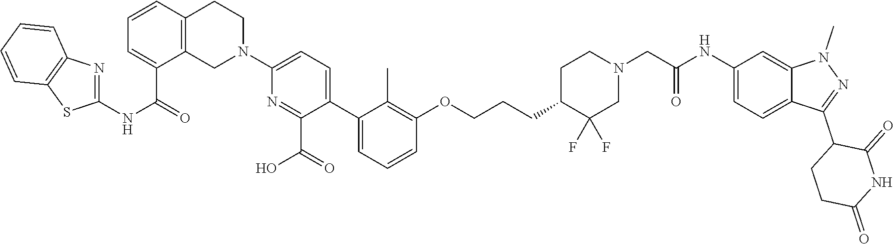 [SMILES: Cc1c(OCCC[C@@H]2CCN(CC(=O)Nc3ccc4c(C5CCC(=O)NC5=O)nn(C)c4c3)CC2(F)F)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-((4R)-1-(2-((3-(2,6-dioxopiperidin-3-yl)-1-methyl-1H-indazol-6-yl)amino)-2-oxoethyl)-3,3-difluoropiperidin-4-yl)propoxy)-2-methylphenyl)picolinic acid

### Step A. Procedure for Preparation of (R)-4-(3-(3-bromo-2-methylphenoxy)propyl)-3,3-difluoropiperidine

To a solution of (R)-tert-butyl 4-(3-(3-bromo-2-methylphenoxy)propyl)-3,3-difluoropiperidine-1-carboxylate (500.00 mg, 1.12 mmol, 1 equiv.) was added HCl/dioxane (4 M, 278.81 μL, 1 equiv.). The mixture was stirred at 25° C. for 2 hours. The reaction mixture was concentrated under reduced pressure to remove solvent. The compound (R)-4-(3-(3-bromo-2-methylphenoxy)propyl)-3,3-difluoropiperidine (350 mg, crude, HCl) was obtained as a white solid.

### Step B. Procedure for Preparation of (R)-ethyl 2-(4-(3-(3-bromo-2-methylphenoxy)propyl)-3,3-difluoropiperidin-1-yl)acetate

To a solution of (R)-4-(3-(3-bromo-2-methylphenoxy)propyl)-3,3-difluoropiperidine (700.00 mg, 2.01 mmol, 1 equiv.) in CH₃CN (10 mL) was added K₂CO₃ (833.46 mg, 6.03 mmol, 3 equiv.) and ethyl 2-bromoacetate (302.13 mg, 1.81 mmol, 200.09 μL, 0.9 equiv.). The mixture was stirred at 60° C. for 1 hour. The reaction mixture was concentrated under reduced pressure to give a residue. The residue was purified by column chromatography (SiO₂, petroleum ether/ethyl acetate 1/0 to 2/1). The compound (R)-ethyl 2-(4-(3-(3-bromo-2-methylphenoxy)propyl)-3,3-difluoropiperidin-1-yl)acetate (600 mg, 1.3 mmol, 68.7% yield, 100% purity) was obtained as a white solid.

MS (ESI) m/z: 436.0 [M+H]⁺

### Step C. Procedure for Preparation of (R)-tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)-3,3-difluoropiperidin-4-yl)propoxy)-2-methylphenyl)picolinate

To a solution of (R)-ethyl 2-(4-(3-(3-bromo-2-methylphenoxy)propyl)-3,3-difluoropiperidin-1-yl)acetate (400 mg, 920.99 μmol, 1 equiv.) in dioxane (10 mL) was added [2-(2-aminophenyl)phenyl]palladium(1+); bis(1-adamantyl)-butyl-phosphane; methanesulfonate (67.07 mg, 92.10 μmol, 0.2 equiv.), K₂CO₃ (1 M, 2.76 mL, 3 equiv.) and tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (564.15 mg, 920.99 μmol, 1 equiv.). The mixture was stirred at 100° C. for 1 hour. The reaction mixture was concentrated under reduced pressure to give a residue. The residue was purified by column chromatography (SiO₂, petroleum ether/ethyl acetate=10/1 to 2/1). The compound (R)-tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)-3,3-difluoropiperidin-4-yl)propoxy)-2-methylphenyl)picolinate (600 mg, 707.1 μmol, 76.8% yield, 99% purity) was obtained as a yellow solid.

MS (ESI) m/z: 840.3 [M+H]⁺

### Step D. Procedure for Preparation of (R)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-3,3-difluoropiperidin-1-yl)acetic acid

To a solution of (R)-tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)-3,3-difluoropiperidin-4-yl)propoxy)-2-methylphenyl)picolinate (600 mg, 714.30 μmol, 1 equiv.) in THE (10 mL) and H₂O (4 mL) was added LiOH·H₂O (51.32 mg, 2.14 mmol, 3 equiv.). The mixture was stirred at 25° C. for 1 hour. The pH was adjusted to 6 with 1 M HCl. The residue was diluted with H₂O (10 mL) and extracted with (10 mL×3). The combined organic layers were washed with aqueous NaCl (10 mL×3), dried over Na₂SO₄, filtered, and concentrated under reduced pressure to give a residue. The compound (R)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-3,3-difluoropiperidin-1-yl)acetic acid (500 mg, crude) was obtained as a yellow solid.

MS (ESI) m/z: 812.8 [M+H]⁺

### Step E. Procedure for Preparation of tert-butyl 6-(8-(benzo[d]thiazol-2-ylcarbamoyl)-3,4-dihydroisoquinolin-2(1H)-yl)-3-(3-(3-((4R)-1-(2-((3-(2,6-dioxopiperidin-3-yl)-1-methyl-11H-indazol-6-yl)amino)-2-oxoethyl)-3,3-difluoropiperidin-4-yl)propoxy)-2-methylphenyl)picolinate

To a solution of (R)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-3,3-difluoropiperidin-1-yl)acetic acid (100.00 mg, 123.16 μmol, 1 equiv.) in DMF (2 mL) was added HATU (56.20 mg, 147.79 μmol, 1.2 equiv.), DIEA (47.75 mg, 369.48 μmol, 64.36 μL, 3 equiv.), and 3-(6-amino-1-methyl-indazol-3-yl)piperidine-2,6-dione (38.17 mg, 147.79 μmol, 1.2 equiv.). The mixture was stirred at 25° C. for 1 hour. The mixture was diluted with H₂O (10 mL) and extracted with DCM (10 mL×3). The combined organic layers were washed with aqueous NaCl (10 mL×3), dried over Na₂SO₄, filtered, and concentrated under reduced pressure to give a residue. The compound tert-butyl 6-(8-(benzo[d]thiazol-2-ylcarbamoyl)-3,4-dihydroisoquinolin-2(1H)-yl)-3-(3-(3-((4R)-1-(2-((3-(2,6-dioxopiperidin-3-yl)-1-methyl-1H-indazol-6-yl)amino)-2-oxoethyl)-3,3-difluoropiperidin-4-yl)propoxy)-2-methylphenyl)picolinate (120 mg, crude) was obtained as a brown oil.

MS (ESI) m/z: 527.2 [(M+2)/2]⁺

### Step F. Procedure for Preparation of 6-(8-(benzo[d]thiazol-2-ylcarbamoyl)-3,4-dihydroisoquinolin-2(1H)-yl)-3-(3-(3-((4R)-1-(2-((3-(2,6-dioxopiperidin-3-yl)-1-methyl-11H-indazol-6-yl)amino)-2-oxoethyl)-3,3-difluoropiperidin-4-yl)propoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(3-((4R)-1-(2-((3-(2,6-dioxopiperidin-3-yl)-1-methyl-1H-indazol-6-yl)amino)-2-oxoethyl)-3,3-difluoropiperidin-4-yl)propoxy)-2-methylphenyl)picolinate (120.00 mg, 114.05 μmol, 1 equiv.) in DCM (1 mL) was added TFA (1.54 g, 13.51 mmol, 1 mL, 118.42 equiv.). The mixture was stirred at 25° C. for 2 hours. The reaction mixture was concentrated under reduced pressure to give a residue. The residue was purified by prep-HPLC. The compound 6-(8-(benzo[d]thiazol-2-ylcarbamoyl)-3,4-dihydroisoquinolin-2(1H)-yl)-3-(3-(3-((4R)-1-(2-((3-(2,6-dioxopiperidin-3-yl)-1-methyl-1H-indazol-6-yl)amino)-2-oxoethyl)-3,3-difluoropiperidin-4-yl)propoxy)-2-methylphenyl)picolinic acid (28.9 mg, 28.3 μmol, 24.8% yield, 97.7% purity) was obtained as a yellow solid.

MS (ESI) m/z: 996.7 [M+H]⁺

¹H NMR (400 MHz, DMSO-d₆) δ 12.87 (d, J=5.2 Hz, 1H), 12.74-12.23 (m, 1H), 10.88 (s, 1H), 10.04-9.85 (m, 1H), 8.08-8.00 (m, 2H), 7.79 (d, J=8.0 Hz, 1H), 7.66-7.59 (m, 2H), 7.50-7.42 (m, 3H), 7.40-7.32 (m, 2H), 7.14-7.06 (m, 2H), 6.97 (d, J=8.8 Hz, 1H), 6.89 (d, J=8.4 Hz, 1H), 6.64 (d, J=7.2 Hz, 1H), 4.98 (s, 2H), 4.32 (dd, J=5.2, 9.6 Hz, 1H), 3.99 (s, 2H), 3.94-3.89 (m, 5H), 3.03 (t, J=5.6 Hz, 2H), 2.74-2.54 (m, 4H), 2.33 (d, J=2.0 Hz, 2H), 2.26-2.06 (m, 2H), 1.94 (s, 1H), 1.92-1.66 (m, 9H), 1.51-1.35 (m, 2H)

### Example 46. Preparation of Compound 193b

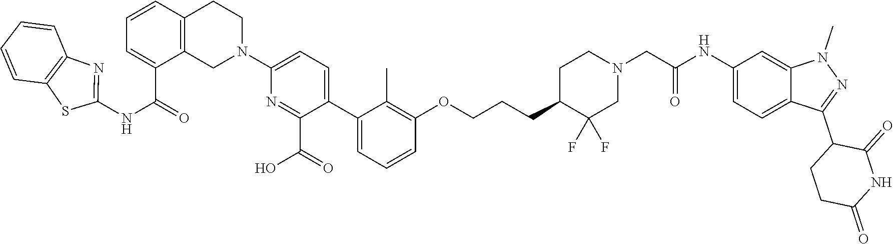 [SMILES: Cc1c(OCCC[C@H]2CCN(CC(=O)Nc3ccc4c(C5CCC(=O)NC5=O)nn(C)c4c3)CC2(F)F)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl-3-(3-(3-((4S)-1-(2-((3-(2,6-dioxopiperidin-3-yl)-1-methyl-1H-indazol-6-yl)amino)-2-oxoethyl)-3,3-difluoropiperidin-4-yl)propoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(3-((4S)-1-(2-((3-(2,6-dioxopiperidin-3-yl)-1-methyl-1H-indazol-6-yl)amino)-2-oxoethyl)-3,3-difluoropiperidin-4-yl)propoxy)-2-methylphenyl)picolinate

To a solution of (S)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-3,3-difluoropiperidin-1-yl)acetic acid (100 mg, 123.16 μmol, 1 equiv.) in DMF (2 mL) was added HATU (56.20 mg, 147.79 μmol, 1.2 equiv.), DIEA (47.75 mg, 369.48 μmol, 64.36 μL, 3 equiv.), and 3-(6-amino-1-methyl-indazol-3-yl) piperidine-2,6-dione (38.17 mg, 147.79 μmol, 1.2 equiv.). The mixture was stirred at 25° C. for 1 hour. The residue was diluted with H₂O (10 mL) and extracted with DCM (10 mL×3). The combined organic layers were washed with aqueous NaCl (10 mL×3), dried over Na₂SO₄, filtered, and concentrated under reduced pressure to give a residue. The compound tert-butyl 6-(8-(benzo[d]thiazol-2-ylcarbamoyl)-3,4-dihydroisoquinolin-2(1H)-yl)-3-(3-(3-((4S)-1-(2-((3-(2,6-dioxopiperidin-3-yl)-1-methyl-1H-indazol-6-yl)amino)-2-oxoethyl)-3,3-difluoropiperidin-4-yl)propoxy)-2-methylphenyl)picolinate (120 mg, crude) was obtained as a yellow oil.

MS (ESI) m/z: 527.1 [(M+2)/2]⁺

### Step B. Procedure for Preparation of 6-(8-(benzo[d]thiazol-2-ylcarbamoyl)-3,4-dihydroisoquinolin-2(1H)-yl)-3-(3-(3-((4S)-1-(2-((3-(2,6-dioxopiperidin-3-yl)-1-methyl-11H-indazol-6-yl)amino)-2-oxoethyl)-3,3-difluoropiperidin-4-yl)propoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(3-((4S)-1-(2-((3-(2,6-dioxopiperidin-3-yl)-1-methyl-1H-indazol-6-yl)amino)-2-oxoethyl)-3,3-difluoropiperidin-4-yl)propoxy)-2-methylphenyl)picolinate (120.00 mg, 114.05 μmol, 1 equiv.) in DCM (1 mL) was added TFA (1.54 g, 13.51 mmol, 1 mL, 118.42 equiv.). The mixture was stirred at 25° C. for 2 hours. The reaction mixture was concentrated under reduced pressure to give a residue. The residue was purified by prep-HPLC. The compound 6-(8-(benzo[d]thiazol-2-ylcarbamoyl)-3,4-dihydroisoquinolin-2(1H)-yl)-3-(3-(3-((4S)-1-(2-((3-(2,6-dioxopiperidin-3-yl)-1-methyl-1H-indazol-6-yl)amino)-2-oxoethyl)-3,3-difluoropiperidin-4-yl)propoxy)-2-methylphenyl)picolinic acid (23.5 mg, 22.7 μmol, 19.8% yield, 95.8% purity) was obtained as a yellow solid.

MS (ESI) m/z: 996.7 [M+H]⁺

¹H NMR (400 MHz, DMSO-d₆) δ=13.01-12.76 (m, 1H), 12.69-12.32 (m, 1H), 10.88 (s, 1H), 10.04 (d, J=12.0 Hz, 1H), 8.06-8.00 (m, 2H), 7.79 (d, J=8.0 Hz, 1H), 7.67-7.59 (m, 2H), 7.48-7.42 (m, 3H), 7.40-7.31 (m, 2H), 7.14-7.06 (m, 2H), 6.97 (d, J=8.8 Hz, 1H), 6.89 (d, J=8.0 Hz, 1H), 6.63 (d, J=7.6 Hz, 1H), 4.98 (s, 2H), 4.32 (dd, J=5.2, 9.6 Hz, 1H), 3.99 (s, 2H), 3.92 (s, 5H), 3.03 (s, 2H), 2.67-2.60 (m, 4H), 2.33 (s, 2H), 2.23-2.10 (m, 2H), 2.06-1.98 (m, 1H), 1.90 (s, 9H), 1.52-1.35 (m, 2H)

### Example 47. Preparation of Compound 197a

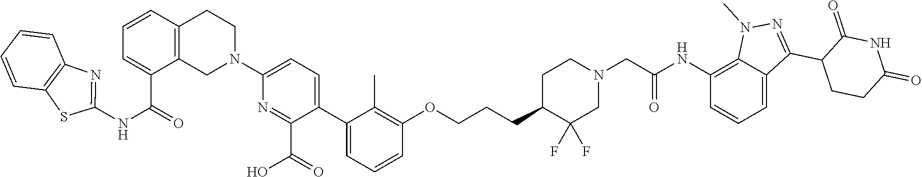 [SMILES: Cc1c(OCCC[C@H]2CCN(CC(=O)Nc3cccc4c(C5CCC(=O)NC5=O)nn(C)c34)CC2(F)F)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-((4S)-1-(2-((3-(2,6-dioxopiperidin-3-yl)-1-methyl-1H-indazol-7-yl)amino)-2-oxoethyl)-3,3-difluoropiperidin-4-yl)propoxy)-2-methylphenyl)picolinic acid

### Step A. Procedure for Preparation of tert-butyl (4E)-4-(3-benzyloxypropylidene)-3,3-difluoro-piperidine-1-carboxylate

A mixture of tert-butyl 3,3-difluoro-4-oxo-piperidine-1-carboxylate (8 g, 34.01 mmol, 1 equiv.) in THE (100 mL) was added NaHMDS (1 M, 44.21 mL, 1.3 equiv.) at −70° C. under N₂ atmosphere. Then the 3-benzyloxypropyl (triphenyl) phosphonium; bromide (25.07 g, 51.01 mmol, 1.5 equiv.) was added the mixture at −70° C. under N₂ atmosphere. The mixture was stirred at 25° C. for 20 hours under N₂ atmosphere. The reaction mixture was quenched by addition NH₄Cl 100 mL at 0° C. Then diluted with H₂O 100 mL and extracted with ethyl acetate 300 mL (100 mL×3). The combined organic layers were washed with aqueous NaCl 300 mL (100 mL×3), dried over Na₂SO₄, filtered and concentrated under reduced pressure to give a residue. The residue was purified by column chromatography (SiO₂, petroleum ether/ethyl acetate=10/1 to 3/1). The compound tert-butyl (4E)-4-(3-benzyloxypropylidene)-3,3-difluoro-piperidine-1-carboxylate (4.6 g, 11.9 mmol, 35.0% yield, 95.2% purity) was obtained as a white oil.

MS (ESI) m/z: 268.3 [M-100]⁺

### Step B. Procedure for Preparation of tert-butyl 3,3-difluoro-4-(3-hydroxypropyl)piperidine-1-carboxylate

To a solution of tert-butyl (4E)-4-(3-benzyloxypropylidene)-3,3-difluoro-piperidine-1-carboxylate (3.6 g, 9.80 mmol, 1 equiv.) in MeOH (50 mL) was added Pd/C (2 g, 19.60 mmol, 10% purity, 0.5 equiv.) and Pd(OH)₂ (2 g, 2.85 mmol, 20% purity, 0.5 equiv.) under N₂ atmosphere. The suspension was degassed and purged with H₂ three times. The mixture was stirred under H₂ (15 psi.) at 25° C. for 5 hours. The reaction mixture was filtered and concentrated under reduced pressure to remove solvent. The compound tert-butyl 3,3-difluoro-4-(3-hydroxypropyl) piperidine-1-carboxylate (2.1 g, crude) was obtained as a white oil.

MS (ESI) m/z: 180.4 [M+H-100]⁺

### Step C. Procedure for Preparation of tert-butyl 4-(3-(3-bromo-2-methylphenoxy)propyl)-3,3-difluoropiperidine-1-carboxylate

To a solution of 3-bromo-2-methyl-phenol (1.41 g, 7.52 mmol, 1 equiv.) in toluene (50 mL) was added 2-(tributyl-λ⁵-phosphanylidene)acetonitrile (2.72 g, 11.28 mmol, 1.5 equiv.) and tert-butyl 3,3-difluoro-4-(3-hydroxypropyl)piperidine-1-carboxylate (2.1 g, 7.52 mmol, 1 equiv.). The suspension was degassed and purged with N₂ three times. The mixture was stirred under N₂ at 120° C. for 2 hours. The reaction mixture was concentrated under reduced pressure to give a residue. The residue was purified by column chromatography (SiO₂, petroleum ether/ethyl acetate=1/0 to 10/1). The compound tert-butyl 4-(3-(3-bromo-2-methylphenoxy)propyl)-3,3-difluoropiperidine-1-carboxylate (2.1 g, 4.4 mmol, 58.5% yield, 94% purity) was obtained as a white solid.

### Step D. Procedure for Preparation of (S)-tert-butyl 4-(3-(3-bromo-2-methylphenoxy)propyl)-3,3-difluoropiperidine-1-carboxylate

The tert-butyl 4-(3-(3-bromo-2-methylphenoxy)propyl)-3,3-difluoropiperidine-1-carboxylate residue was purified by prep-HPLC. The compound (S)-tert-butyl 4-(3-(3-bromo-2-methylphenoxy)propyl)-3,3-difluoropiperidine-1-carboxylate (1 g, 2.2 mmol, 47.1% yield, 99% purity) was obtained as a white oil.

MS (ESI) m/z: 348.3 [M+H-100]⁺

¹H NMR (400 MHz, DMSO-d₆) δ=7.18-7.13 (m, 1H), 7.09 (t, J=2.4, 8.0 Hz, 1H), 6.97 (d, J=8.0 Hz, 1H), 4.13-3.95 (m, 4H), 3.90 (d, J=11.2 Hz, 1H), 3.16-2.69 (m, 2H), 2.23 (d, J=2.4 Hz, 3H), 1.99 (d, J=3.2 Hz, 1H), 1.85 (d, J=8.4 Hz, 5H), 1.39 (d, J=2.4 Hz, 9H)

### Step E. Procedure for Preparation of (S)-4-(3-(3-bromo-2-methylphenoxy)propyl)-3,3-difluoropiperidine

To a solution of (S)-tert-butyl 4-(3-(3-bromo-2-methylphenoxy)propyl)-3,3-difluoropiperidine-1-carboxylate (1 g, 2.23 mmol, 1 equiv.) was added HCl/dioxane (4 M, 557.61 μL, 1 equiv.). The mixture was stirred at 25° C. for 2 hours. The reaction mixture was concentrated under reduced pressure to remove solvent. The compound (S)-4-(3-(3-bromo-2-methylphenoxy)propyl)-3,3-difluoropiperidine (700 mg, crude) was obtained as a white solid.

### Step F. Procedure for Preparation of (S)-ethyl 2-(4-(3-(3-bromo-2-methylphenoxy)propyl)-3,3-difluoropiperidin-1-yl)acetate

To a solution of (S)-4-(3-(3-bromo-2-methylphenoxy)propyl)-3,3-difluoropiperidine (700 mg, 2.01 mmol, 1 equiv.) in CH₃CN (10 mL) was added K₂CO₃ (833.46 mg, 6.03 mmol, 3 equiv.) and ethyl 2-bromoacetate (302.13 mg, 1.81 mmol, 200.09 μL, 0.9 equiv.). The mixture was stirred at 60° C. for 1 hour. The reaction mixture was concentrated under reduced pressure to give a residue. The residue was purified by column chromatography (SiO₂, petroleum ether/ethyl acetate, from 1/0 to 2/1). The compound (S)-ethyl 2-(4-(3-(3-bromo-2-methylphenoxy)propyl)-3,3-difluoropiperidin-1-yl)acetate (600 mg, 1.4 mmol, 68.7% yield) was obtained as a white solid.

MS (ESI) m/z: 436.0 [M+H]⁺

### Step G. Procedure for Preparation of (S)-tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)-3,3-difluoropiperidin-4-yl)propoxy)-2-methylphenyl)picolinate

To a solution of (S)-ethyl 2-(4-(3-(3-bromo-2-methylphenoxy)propyl)-3,3-difluoropiperidin-1-yl)acetate (400 mg, 920.99 μmol, 1 equiv.) in dioxane (10 mL) was added [2-(2-aminophenyl)phenyl]palladium(1+); bis(1-adamantyl)-butyl-phosphane; methanesulfonate (67.07 mg, 92.10 μmol, 0.1 equiv.), K₂CO₃ (1.5 M, 3760 μL, 3 equiv.), and tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (564.15 mg, 920.99 μmol, 1 equiv.). The mixture was stirred at 100° C. for 1 hour. The reaction mixture was concentrated under reduced pressure to give a residue. The residue was purified by column chromatography (SiO₂, petroleum ether/ethyl acetate, 10/1 to 2/1). The compound (S)-tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)-3,3-difluoropiperidin-4-yl)propoxy)-2-methylphenyl)picolinate (670 mg, 789.6 μmol, 85.7% yield, 99% purity) was obtained as a yellow solid.

MS (ESI) m/z: 840.4 [M+H]⁺

### Step H. Procedure for Preparation of (S)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-3,3-difluoropiperidin-1-yl)acetic acid

To a solution of (S)-tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)-3,3-difluoropiperidin-4-yl)propoxy)-2-methylphenyl)picolinate (580 mg, 690.49 μmol, 1 equiv.) in THE (10 mL) and H₂O (4 mL) was added LiOH·H₂O (49.61 mg, 2.07 mmol, 3 equiv.). The mixture was stirred at 25° C. for 1 hour. The pH was adjusted to 6 with 1M HCl. The residue was diluted with H₂O (10 mL) and extracted with (10 mL×3). The combined organic layers were washed with aqueous NaCl (10 mL×3), dried over Na₂SO₄, filtered, and concentrated under reduced pressure to give a residue. The compound (S)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-3,3-difluoropiperidin-1-yl)acetic acid (430 mg, crude) was obtained as a yellow solid.

MS (ESI) m/z: 812.5 [M+H]⁺

### Step L Procedure for preparation of tert-butyl 6-(8-(benzo[d]thiazol-2-ylcarbamoyl)-3,4-dihydroisoquinolin-2(1H)-yl)-3-(3-(3-((4S)-1-(2-((3-(2,6-dioxopiperidin-3-yl)-1-methyl-11H-indazol-7-yl)amino)-2-oxoethyl)-3,3-difluoropiperidin-4-yl)propoxy)-2-methylphenyl)picolinate

To a solution of (S)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-3,3-difluoropiperidin-1-yl)acetic acid (100 mg, 123.16 μmol, 1 equiv.) in DMF (2 mL) was added HATU (56.20 mg, 147.80 μmol, 1.2 equiv.), DIEA (47.75 mg, 369.49 μmol, 64.36 μL, 3 equiv.), and 3-(7-amino-1-methyl-indazol-3-yl) piperidine-2,6-dione (38.17 mg, 147.80 μmol, 1.2 equiv.). The mixture was stirred at 25° C. for 1 hour. The residue was diluted with H₂O (10 mL) and extracted with DCM (10 mL×3). The combined organic layers were washed with aqueous NaCl (10 mL×3), dried over Na₂SO₄, filtered, and concentrated under reduced pressure to give a residue. The compound tert-butyl 6-(8-(benzo[d]thiazol-2-ylcarbamoyl)-3,4-dihydroisoquinolin-2(1H)-yl)-3-(3-(3-((4S)-1-(2-((3-(2,6-dioxopiperidin-3-yl)-1-methyl-1H-indazol-7-yl)amino)-2-oxoethyl)-3,3-difluoropiperidin-4-yl)propoxy)-2-methylphenyl)picolinate (120 mg, crude) was obtained as a brown oil.

MS (ESI) m/z: 527.0 [M/2+H]⁺

### Step J. Procedure for Preparation of 6-(8-(benzo[d]thiazol-2-ylcarbamoyl)-3,4-dihydroisoquinolin-2(1H)-yl)-3-(3-(3-((4S)-1-(2-((3-(2,6-dioxopiperidin-3-yl)-1-methyl-11H-indazol-7-yl)amino)-2-oxoethyl)-3,3-difluoropiperidin-4-yl)propoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(3-((45)-1-(2-((3-(2,6-dioxopiperidin-3-yl)-1-methyl-1H-indazol-7-yl)amino)-2-oxoethyl)-3,3-difluoropiperidin-4-yl)propoxy)-2-methylphenyl)picolinate (120.00 mg, 114.05 μmol, 1 equiv.) in DCM (1.5 mL) was added TFA (2.31 g, 20.26 mmol, 1.5 mL, 177.64 equiv.). The mixture was stirred at 25° C. for 2 hours. The reaction mixture was concentrated under reduced pressure to give a residue. The residue was purified by prep-HPLC. The compound 6-(8-(benzo[d]thiazol-2-ylcarbamoyl)-3,4-dihydroisoquinolin-2(1H)-yl)-3-(3-(3-((4S)-1-(2-((3-(2,6-dioxopiperidin-3-yl)-1-methyl-1H-indazol-7-yl)amino)-2-oxoethyl)-3,3-difluoropiperidin-4-yl)propoxy)-2-methylphenyl)picolinic acid (29.1 mg, 28.9 μmol, 25.3% yield, 99.0% purity) was obtained as a yellow solid.

MS (ESI) m/z: 996.7 [M+H]⁺

¹H NMR (400 MHz, DMSO-d₆) δ=12.90-12.80 (m, 1H), 12.71-12.47 (m, 1H), 10.90 (s, 1H), 9.89-9.81 (m, 1H), 8.03 (d, J=8.0 Hz, 1H), 7.79 (d, J=7.6 Hz, 1H), 7.64-7.56 (m, 2H), 7.49-7.43 (m, 3H), 7.40-7.33 (m, 2H), 7.28 (d, J=7.2 Hz, 1H), 7.09 (q, J=7.6 Hz, 2H), 6.97 (d, J=8.8 Hz, 1H), 6.89 (d, J=8.0 Hz, 1H), 6.63 (d, J=8.0 Hz, 1H), 4.98 (s, 2H), 4.37 (dd, J=4.8, 10.0 Hz, 1H), 4.08 (s, 3H), 3.99 (s, 2H), 3.92 (s, 2H), 3.03 (s, 2H), 2.67 (s, 4H), 2.33 (d, J=2.0 Hz, 2H), 2.23-2.08 (m, 2H), 2.06-1.96 (m, 1H), 1.96-1.75 (m, 9H), 1.53-1.36 (m, 2H)

### Example 48. Preparation of Compound 197b

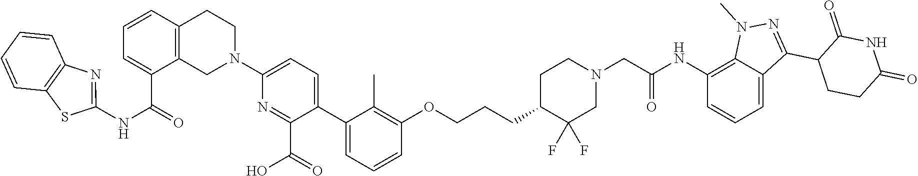 [SMILES: Cc1c(OCCC[C@@H]2CCN(CC(=O)Nc3cccc4c(C5CCC(=O)NC5=O)nn(C)c34)CC2(F)F)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-((4R)-1-(2-((3-(2,6-dioxopiperidin-3-yl)-1-methyl-1H-indazol-7-yl)amino)-2-oxoethyl)-3,3-difluoropiperidin-4-yl)propoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(3-((4R)-1-(2-((3-(2,6-dioxopiperidin-3-yl)-1-methyl-1H-indazol-7-yl)amino)-2-oxoethyl)-3,3-difluoropiperidin-4-yl)propoxy)-2-methylphenyl)picolinate

To a solution of (R)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-3,3-difluoropiperidin-1-yl)acetic acid (100.00 mg, 123.16 μmol, 1 equiv.) in DMF (2 mL) was added HATU (56.20 mg, 147.80 μmol, 1.2 equiv.), DIEA (47.75 mg, 369.49 μmol, 64.36 μL, 3 equiv.), and 3-(7-amino-1-methyl-indazol-3-yl) piperidine-2,6-dione (38.17 mg, 147.80 μmol, 1.2 equiv.). The mixture was stirred at 25° C. for 1 hour. The residue was diluted with H₂O (10 mL) and extracted with DCM (10 mL×3). The combined organic layers were washed with aqueous NaCl (10 mL×3), dried over Na₂SO₄, filtered, and concentrated under reduced pressure to give a residue. The compound tert-butyl 6-(8-(benzo[d]thiazol-2-ylcarbamoyl)-3,4-dihydroisoquinolin-2(1H)-yl)-3-(3-(3-((4R)-1-(2-((3-(2,6-dioxopiperidin-3-yl)-1-methyl-1H-indazol-7-yl)amino)-2-oxoethyl)-3,3-difluoropiperidin-4-yl)propoxy)-2-methylphenyl)picolinate (120 mg, crude) was obtained as a brown oil.

MS (ESI) m/z: 527.2 [(M+2)/2]⁺

### Step B. Procedure for Preparation of 6-(8-(benzo[d]thiazol-2-ylcarbamoyl)-3,4-dihydroisoquinolin-2(1H)-yl)-3-(3-(3-((4R)-1-(2-((3-(2,6-dioxopiperidin-3-yl)-1-methyl-11H-indazol-7-yl)amino)-2-oxoethyl)-3,3-difluoropiperidin-4-yl)propoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(3-((4R)-1-(2-((3-(2,6-dioxopiperidin-3-yl)-1-methyl-1H-indazol-7-yl)amino)-2-oxoethyl)-3,3-difluoropiperidin-4-yl)propoxy)-2-methylphenyl)picolinate (120.00 mg, 114.05 μmol, 1 equiv.) in DCM (1.5 mL) was added TFA (2.31 g, 20.26 mmol, 1.5 mL, 177.64 equiv.). The mixture was stirred at 25° C. for 2 hours. The reaction mixture was concentrated under reduced pressure to give a residue. The residue was purified by prep-HPLC. The compound 6-(8-(benzo[d]thiazol-2-ylcarbamoyl)-3,4-dihydroisoquinolin-2(1H)-yl)-3-(3-(3-((4R)-1-(2-((3-(2,6-dioxopiperidin-3-yl)-1-methyl-1H-indazol-7-yl)amino)-2-oxoethyl)-3,3-difluoropiperidin-4-yl)propoxy)-2-methylphenyl)picolinic acid (27.7 mg, 27.2 μmol, 23.8% yield, 97.8% purity) was obtained as a yellow solid.

MS (ESI) m/z: 996.7 [M+H]⁺

¹H NMR (400 MHz, DMSO-d₆) δ=12.87 (d, J=5.2 Hz, 1H), 12.74-12.23 (m, 1H), 10.88 (s, 1H), 10.04-9.85 (m, 1H), 8.08-8.00 (m, 2H), 7.79 (d, J=8.0 Hz, 1H), 7.66-7.59 (m, 2H), 7.50-7.42 (m, 3H), 7.40-7.32 (m, 2H), 7.14-7.06 (m, 2H), 6.97 (d, J=8.8 Hz, 1H), 6.89 (d, J=8.4 Hz, 1H), 6.64 (d, J=7.2 Hz, 1H), 4.98 (s, 2H), 4.32 (dd, J=5.2, 9.6 Hz, 1H), 3.99 (s, 2H), 3.94-3.89 (m, 5H), 3.03 (t, J=5.6 Hz, 2H), 2.74-2.54 (m, 4H), 2.33 (d, J=2.0 Hz, 2H), 2.26-2.06 (m, 2H), 1.94 (s, 1H), 1.92-1.66 (m, 9H), 1.51-1.35 (m, 2H)

### Example 49. Preparation of Compound 200

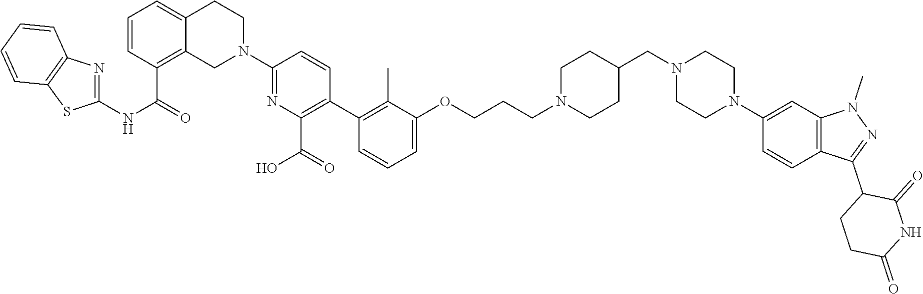 [SMILES: Cc1c(OCCCN2CCC(CN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[4-[[4-[3-(2,6-dioxo-3-piperidyl)-1-methyl-indazol-6-yl]piperazin-1-yl]methyl]-1-piperidyl]propoxy]-2-methyl-phenyl] pyridine-2-carboxylic acid

### Step A. Procedure for Preparation of methyl piperidine-4-carboxylate

To a solution 1-(tert-butyl) 4-methyl piperidine-1,4-dicarboxylate (10 g, 41.10 mmol, 1 equiv.) in DCM (10 mL) and HCl/dioxane (4 M, 10 mL, 9.73e-1 equiv.) was stirred at 25° C. for 2 hours. The reaction mixture was filtered and concentrated under reduced pressure to give a residue. The compound methyl piperidine-4-carboxylate (7.7 g, crude) was obtained as a white solid.

### Step B. Procedure for Preparation of 1-bromo-3-(3-bromopropoxy)-2-methyl-benzene

A mixture of 3-bromo-2-methyl-phenol (2 g, 10.69 mmol, 1 equiv.), 1,3-dibromopropane (10.79 g, 53.47 mmol, 5.45 mL, 5 equiv.), Cs₂CO₃ (10.45 g, 32.08 mmol, 3 equiv.), and KI (177.51 mg, 1.07 mmol, 0.1 equiv.) in CH₃CN (15 mL) was stirred at 25° C. for 5 hours. The reaction mixture was filtered and concentrated under reduced pressure to give a residue. The residue was purified by flash silica gel chromatography (Eluent of 0-20% ethyl acetate/Petroleum). The compound 1-bromo-3-(3-bromopropoxy)-2-methyl-benzene (2.2 g, 7.1 mmol, 66.8% yield) was obtained as a colorless oil.

¹H NMR (400 MHz, DMSO-d₆) δ=7.19-7.15 (m, 1H), 7.10 (t, J=8.0 Hz, 1H), 6.99 (d, J=8.0 Hz, 1H), 4.09 (t, J=5.6 Hz, 2H), 3.69 (t, J=6.4 Hz, 2H), 2.34-2.18 (m, 6H)

### Step C. Procedure for Preparation of methyl 1-[3-(3-bromo-2-methyl-phenoxy) propyl]piperidine-4-carboxylate

To a solution of 1-bromo-3-(3-bromopropoxy)-2-methyl-benzene (1.82 g, 5.91 mmol, 1 eq) in CH₃CN (20 mL) was added methyl piperidine-4-carboxylate (1.17 g, 6.50 mmol, 1.1 equiv. HCl), K₂CO₃ (2.45 g, 17.73 mmol, 3 eq) and KI (98.09 mg, 590.89 μmol, 0.1 eq) at 25° C. The reaction mixture was stirred at 60° C. for 12 hours. The reaction mixture was filtered and concentrated under reduced pressure to give a residue. The residue was purified by flash silica gel chromatography (Eluent of 0˜100% ethyl acetate/petroleum ether). The compound methyl 1-[3-(3-bromo-2-methyl-phenoxy) propyl]piperidine-4-carboxylate (1.9 g, 5.0 mmol, 85.1% yield, 98% purity) was obtained as a colorless oil.

MS (ESI) m/z: 370.2 [M+H]⁺.

### Step D. Procedure for Preparation of [1-[3-(3-bromo-2-methyl-phenoxy) propyl]-4-piperidyl]methanol

To a solution of methyl 1-[3-(3-bromo-2-methyl-phenoxy) propyl]piperidine-4-carboxylate (1.9 g, 5.13 mmol, 1 equiv.) in THE (20 mL) was added LiAlH₄ (214.23 mg, 5.64 mmol, 1.1 equiv.) at 0° C. The reaction mixture was stirred at 0° C. for 1 hour. The reaction mixture was quenched with Na₂SO₄·10 H₂O (20 mg) and slowly warmed to 20° C. The mixture was filtered and washed with DCM (50 mL). Then the organic layers were concentrated under reduced pressure to give [1-[3-(3-bromo-2-methyl-phenoxy) propyl]-4-piperidyl]methanol (1.55 g, crude) as a colorless oil.

MS (ESI) m/z: 342.2 [M+H]⁺.

### Step E. Procedure for Preparation of 1-[3-(3-bromo-2-methyl-phenoxy) propyl]piperidine-4-carbaldehyde

A solution of DMSO (1.23 g, 15.78 mmol, 1.23 mL, 4 equiv.) in DCM (10 mL) was added dropwise to a solution of oxalyl dichloride (1.00 g, 7.89 mmol, 690.54 μL, 2 equiv.) in DCM (1 mL) at −70° C. under N₂ atmosphere. The mixture was stirred at −70° C. for 30 minutes. After which time [1-[3-(3-bromo-2-methyl-phenoxy) propyl]-4-piperidyl]methanol (1.35 g, 3.94 mmol, 1 equiv.) in DCM (1 mL) was added dropwise at −70° C. The solution was stirred for 30 minutes at −70° C. Then TEA (2.39 g, 23.67 mmol, 3.29 mL, 6 equiv.) was added into the solution. The solution was stirred at 25° C. for 1 hour under N₂ atmosphere. The reaction mixture was diluted with water (10 mL) and extracted with DCM (10 mL×3). The combined organic layers were washed with brine (10 mL×1), dried over Na₂SO₄, filtered, and concentrated under reduced pressure to give a residue. The residue was purified by column chromatography (SiO₂, petroleum ether/ethyl acetate=20/1 to 10/1). The compound 1-[3-(3-bromo-2-methyl-phenoxy) propyl]piperidine-4-carbaldehyde (1.4 g, crude) was obtained as a yellow oil.

¹H NMR (400 MHz, CDCl₃-d) δ=9.68-9.63 (m, 1H), 7.13 (d, J=8.0 Hz, 1H), 6.98 (t, J=8.0 Hz, 1H), 6.76 (d, J=8.0 Hz, 1H), 4.01-3.97 (m, 2H), 2.91-2.81 (m, 2H), 2.56-2.51 (m, 2H), 2.30 (s, 3H), 2.28-2.22 (m, 1H), 2.17-2.09 (m, 2H), 2.03-1.95 (m, 2H), 1.94-1.87 (m, 2H), 1.76-1.64 (m, 2H)

### Step F. Procedure for Preparation of 3-[6-[4-[[1-[3-(3-bromo-2-methyl-phenoxy) propyl]-4-piperidyl]methyl]piperazin-1-yl]-1-methyl-indazol-3-yl]piperidine-2,6-dione

To a solution of 1-[3-(3-bromo-2-methyl-phenoxy) propyl]piperidine-4-carbaldehyde (200 mg, 587.80 μmol, 1 equiv.) in DCM (2 mL) was added 3-(1-methyl-6-piperazin-1-yl-indazol-3-yl) piperidine-2,6-dione (106.93 mg, 293.90 μmol, 0.5 equiv., HCl) and AcOH (3.53 mg, 58.78 μmol, 0.1 equiv.) at 25° C. The mixture was stirred at 25° C. for 18 hours. NaBH(OAc)₃ (373.73 mg, 1.76 mmol, 3 equiv.) was added to the mixture at 25° C. The reaction mixture was stirred at 25° C. for 2 hours. The reaction mixture was filtered and concentrated under reduced pressure to give a residue. The residue was purified by prep-TLC (SiO₂, DCM:MeOH=20:1). The compound 3-[6-[4-[[1-[3-(3-bromo-2-methyl-phenoxy) propyl]-4-piperidyl]methyl] piperazin-1-yl]-1-methyl-indazol-3-yl]piperidine-2,6-dione (100 mg, 135.0 μmol, 22.9% yield, 88% purity) was obtained as a yellow oil.

MS (ESI) m/z: 653.0 [M+H]⁺.

### Step G. Procedure for Preparation of tert-butyl 6-[8-(1,3-benzothiazol-2-ylcarbamoyl)-3,4-dihydro-1H-isoquinolin-2-yl]-3-[3-[3-[4-[[4-[3-(2,6-dioxo-3-piperidyl)-1-methyl-indazol-6-yl]piperazin-1-yl]methyl]-1-piperidyl]propoxy]-2-methyl-phenyl]pyridine-2-carboxylate

3-[6-[4-[[1-[3-(3-bromo-2-methyl-phenoxy) propyl]-4-piperidyl]methyl]piperazin-1-yl]-1-methyl-indazol-3-yl]piperidine-2,6-dione (100 mg, 153.46 μmol, 1 equiv.), tert-butyl 6-[8-(1,3-benzothiazol-2-ylcarbamoyl)-3,4-dihydro-1H-isoquinolin-2-yl]-3-(4, 4, 5,5-tetramethyl-1, 3,2-dioxaborolan-2-yl) pyridine-2-carboxylate (122.20 mg, 199.50 μmol, 1.3 equiv.), KF (26.75 mg, 460.38 μmol, 10.79 μL, 3 equiv.) and [2-(2-aminophenyl) phenyl]palladium (1+); bis (1-adamantyl)-butyl-phosphane; methanesulfonate (11.18 mg, 15.35 μmol, 0.1 equiv.) were taken up into a microwave tube in dioxane (1 mL) and H₂O (0.1 mL). The sealed tube was heated at 100° C. for 1 hour under microwave. The reaction mixture was filtered and concentrated under reduced pressure to give a residue. The residue was purified by prep-TLC (SiO₂, DCM:MeOH=10:1). The compound tert-butyl 6-[8-(1,3-benzothiazol-2-ylcarbamoyl)-3,4-dihydro-1H-isoquinolin-2-yl]-3-[3-[3-[4-[[4-[3-(2,6-dioxo-3-piperidyl)-1-methyl-indazol-6-yl]piperazin-1-yl]methyl]-1-piperidyl]propoxy]-2-methyl-phenyl]pyridine-2-carboxylate (40 mg, 34.5 μmol, 22.5% yield, 91.2% purity) was obtained as a yellow oil.

MS (ESI) m/z: 1057.4 [M+H]⁺.

### Step H. Procedure for Preparation of 6-[8-(1,3-benzothiazol-2-ylcarbamoyl)-3,4-dihydro-1H-isoquinolin-2-yl]-3-[3-[3-[4-[[4-[3-(2,6-dioxo-3-piperidyl)-1-methyl-indazol-6-yl]piperazin-1-yl]methyl]-1-piperidyl]propoxy]-2-methyl-phenyl] pyridine-2-carboxylic acid

A solution of tert-butyl 6-[8-(1,3-benzothiazol-2-ylcarbamoyl)-3,4-dihydro-1H-isoquinolin-2-yl]-3-[3-[3-[4-[[4-[3-(2,6-dioxo-3-piperidyl)-1-methyl-indazol-6-yl]piperazin-1-yl]methyl]-1-piperidyl]propoxy]-2-methyl-phenyl]pyridine-2-carboxylate (40 mg, 37.83 μmol, 1 equiv.) in DCM (0.5 mL) and TFA (0.5 mL) was stirred at 25° C. for 18 hours. The reaction mixture was filtered and concentrated under reduced pressure to give a residue. The residue was purified by prep-HPLC. The compound 6-[8-(1, 3-benzothiazol-2-ylcarbamoyl)-3, 4-dihydro-1H-isoquinolin-2-yl]-3-[3-[3-[4-[[4-[3-(2, 6-dioxo-3-piperidyl)-1-methyl-indazol-6-yl]piperazin-1-yl]methyl]-1-piperidyl]propoxy]-2-methyl-phenyl] pyridine-2-carboxylic acid (21.1 mg, 20.9 μmol, 55.4% yield, 99.3% purity) was obtained as a pink solid.

MS (ESI) m/z: 1001.4 [M+H]⁺.

¹H NMR (400 MHz, DMSO-d₆) δ=10.85 (s, 1H), 8.03 (d, J=8.0 Hz, 1H), 7.79 (d, J=8.0 Hz, 1H), 7.62 (d, J=8.0 Hz, 1H), 7.52-7.31 (m, 6H), 7.14-7.06 (m, 1H), 6.96 (d, J=8.4 Hz, 1H), 6.93-6.86 (m, 2H), 6.83 (s, 1H), 6.65 (d, J=7.6 Hz, 1H), 4.98 (s, 2H), 4.25 (dd, J=4.8, 8.8 Hz, 1H), 4.04-3.99 (m, 2H), 3.93-3.86 (m, 5H), 3.22-3.20 (m, 4H), 3.04-2.99 (m, 4H), 2.90-2.81 (m, 4H), 2.67 (s, 1H), 2.63-2.58 (m, 2H), 2.33-2.28 (m, 2H), 2.23-2.19 (m, 2H), 2.18-2.11 (m, 2H), 2.06-1.97 (m, 3H), 1.91 (s, 3H), 1.80 (d, J=12.8 Hz, 2H), 1.69 (d, J=3.2 Hz, 1H), 1.29-1.17 (m, 2H)

### Example 50. Preparation of Compound 202a

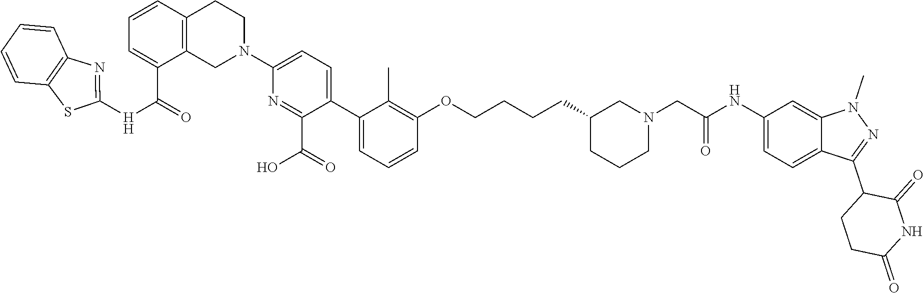

### 6-[8-(1,3-benzothiazol-2-ylcarbamoyl)-3,4-dihydro-1H-isoquinolin-2-yl]-3-[3-[4-[(3S)-1-[2-[[3-(2,6-dioxo-3-piperidyl)-1-methyl-indazol-6-yl]amino]-2-oxo-ethyl]-3-piperidyl]butoxy]-2-methyl-phenyl]pyridine-2-carboxylic acid

### Step A. Procedure for Preparation of tert-butyl (3S)-3-formylpiperidine-1-carboxylate

A mixture of tert-butyl (3S)-3-(hydroxymethyl)piperidine-1-carboxylate (4 g, 18.58 mmol, 1 equiv.) and DMP (8.67 g, 20.44 mmol, 6.33 mL, 1.1 equiv.) in DCM (50 mL) was stirred at 20° C. for 1 hour under N₂ atmosphere. The mixture was filtered and concentrated under reduced pressure to give a residue. The residue was purified by column chromatography (SiO₂, petroleum ether/ethyl acetate=50/1 to 3/1) to give tert-butyl (3S)-3-formylpiperidine-1-carboxylate (3 g, 14.0 mmol, 75.7% yield) as a colorless oil.

¹H NMR ¹H NMR (400 MHz, DMSO-d₆) δ=9.59 (s, 1H), 3.72 (d, J=8.0 Hz, 1H), 3.43 (s, 1H), 3.14-3.01 (m, 1H), 2.49-2.41 (m, 1H), 1.89-1.80 (m, 1H), 1.65-1.48 (m, 2H), 1.39 (s, 11H)

### Step B. Procedure for Preparation of tert-butyl (3R)-3-[(E)-4-benzyloxybut-1-enyl]piperidine-1-carboxylate

A mixture of 3-benzyloxypropyl(triphenyl)phosphonium; bromide (3.46 g, 7.03 mmol, 1 equiv.) in THE (5 mL) was degassed and purged with N₂ three times and cooled to −70° C. LiHMDS (1 M, 10.55 mL, 1.5 equiv.) was added dropwise at −70° C. After stirring at −70° C. for 1 hour, tert-butyl (3S)-3-formylpiperidine-1-carboxylate (1.5 g, 7.03 mmol, 1 equiv.) in THE (3 mL) was added dropwise at −70° C. The mixture was stirred at −70° C. for 1 hour and was quenched by addition of saturated NH₄Cl solution 20 mL dropwise at 0° C. The resulting mixture was extracted with EtOAc (20 mL×3). The combined organic layers were washed with brine (10 mL×3), dried over anhydrous Na₂SO₄, filtered, and concentrated under reduced pressure to give a residue. The residue was purified by column chromatography (SiO₂, petroleum ether/ethyl acetate 200/1 to 10/1) to give tert-butyl (3R)-3-[(E)-4-benzyloxybut-1-enyl]piperidine-1-carboxylate (1.8 g, 4.6 mmol, 65.9% yield, 89% purity) as a colorless oil.

¹H NMR (400 MHz, DMSO-d₆) δ=7.39-7.18 (m, 5H), 5.48-5.36 (m, 1H), 5.28-5.18 (m, 1H), 4.46 (s, 2H), 3.84-3.64 (m, 2H), 3.44 (t, J=6.8 Hz, 2H), 2.72 (s, 1H), 2.63-2.51 (m, 1H), 2.41-2.25 (m, 3H), 1.66 (d, J=12.4 Hz, 1H), 1.60-1.51 (m, 1H), 1.38 (s, 10H), 1.28-1.17 (m, 1H)

### Step C. Procedure for Preparation of tert-butyl (3S)-3-(4-hydroxybutyl)piperidine-1-carboxylate

A mixture of tert-butyl (3R)-3-[(E)-4-benzyloxybut-1-enyl]piperidine-1-carboxylate (1.8 g, 5.21 mmol, 1 equiv.), Pd/C (0.1 g, 521.02 μmol, 10% purity, 0.1 equiv.) and Pd(OH)₂ (0.1 g, 71.21 μmol, 10% purity, 0.01.37 equiv.) in MeOH (200 mL) was degassed and purged with H₂ (15 Psi) three times, and then the mixture was stirred at 20° C. for 16 hours under H₂ (15 Psi) atmosphere. The mixture was filtered and concentrated under reduced pressure to give crude tert-butyl (3S)-3-(4-hydroxybutyl)piperidine-1-carboxylate (1.3 g, 4.0 mmol, 77.5% yield, 80% purity) as a gray oil.

¹H NMR (400 MHz, DMSO-d₆) δ=3.72 (s, 2H), 3.38 (q, J=6.0 Hz, 1H), 2.84-2.70 (m, 1H), 2.50 (s, 1H), 1.74 (d, J=10.4 Hz, 1H), 1.62-1.50 (m, 1H), 1.38 (s, 11H), 1.34-1.11 (m, 7H), 1.08 (d, J=7.6 Hz, 1H), 0.87 (t, J=6.4 Hz, 1H)

### Step D. Procedure for Preparation of tert-butyl (3S)-3-[4-(3-bromo-2-methyl-phenoxy)butyl]piperidine-1-carboxylate

A mixture of 3-bromo-2-methyl-phenol (399.69 mg, 2.14 mmol, 1.1 equiv.), tert-butyl (3S)-3-(4-hydroxybutyl)piperidine-1-carboxylate (0.5 g, 1.94 mmol, 1 equiv.) in toluene (5 mL) was degassed and purged with N₂ three times, and then 2-(tributyl-λ⁵-phosphanylidene)acetonitrile (515.78 mg, 2.14 mmol, 2.14 mL, 1.1 equiv.) was added dropwise. The mixture was stirred at 110° C. for 16 hours under N₂ atmosphere. The reaction mixture was concentrated under reduced pressure to give a residue. The residue was purified by flash silica gel chromatography (Eluent of 0˜25% ethyl acetate/petroleum ether) to give tert-butyl (3S)-3-[4-(3-bromo-2-methyl-phenoxy)butyl]piperidine-1-carboxylate (0.5 g, 1.1 mmol, 55.5% yield, 92% purity) as a colorless oil.

MS (ESI) m/z: 326.2 [M-100+H]+

### Step E. Procedure for Preparation of (3S)-3-[4-(3-bromo-2-methyl-phenoxy)butyl]piperidine

To a solution of tert-butyl (3S)-3-[4-(3-bromo-2-methyl-phenoxy)butyl]piperidine-1-carboxylate (0.5 g, 1.17 mmol, 1 equiv.) in HCl/dioxane (5 mL) was stirred at 20° C. for 1 hour. The reaction mixture was concentrated under reduced pressure to give a residue. The crude product was triturated with petroleum ether/ethyl acetate=5:1 (2 mL) at 20° C. for 30 min to give (3S)-3-[4-(3-bromo-2-methyl-phenoxy)butyl]piperidine (0.3 g, 919.4 μmol, 78.4% yield) as a white solid.

¹H NMR (400 MHz, DMSO-d₆) δ=8.81-8.58 (m, 1H), 8.52-8.30 (m, 1H), 7.19-7.14 (m, 1H), 7.13-7.07 (m, 1H), 6.97 (d, J=7.6 Hz, 1H), 3.99 (br t, J=6.4 Hz, 2H), 2.83-2.68 (m, 2H), 2.24 (s, 3H), 1.82-1.71 (m, 4H), 1.63-1.55 (m, 1H), 1.50-1.41 (m, 2H), 1.35-1.22 (m, 5H), 1.18-1.05 (m, 1H)

### Step F. Procedure for Preparation of ethyl 2-[(3S)-3-[4-(3-bromo-2-methyl-phenoxy)butyl]-1-piperidyl]acetate

To a solution of ethyl 2-bromoacetate (91.16 mg, 545.86 μmol, 60.37 μL, 0.9 equiv.) and (3S)-3-[4-(3-bromo-2-methyl-phenoxy)butyl]piperidine (220 mg, 606.51 μmol, 1 equiv., HCl) in CH₃CN (2 mL) was added K₂CO₃ (251.47 mg, 1.82 mmol, 3 equiv.). The mixture was stirred at 60° C. for 1 hour. The reaction mixture was filtered and concentrated under reduced pressure to give a residue. The residue was purified by flash silica gel chromatography (Eluent of 0˜25% ethyl acetate/petroleum ether) to give ethyl 2-[(3S)-3-[4-(3-bromo-2-methyl-phenoxy)butyl]-1-piperidyl]acetate (0.18 g, 436.5 μmol, 71.9% yield) as a light yellow oil

¹H NMR (400 MHz, CDCl₃) δ=7.06 (d, J=8.0 Hz, 1H), 6.91 (t, J=8.0 Hz, 1H), 6.68 (d, J=8.0 Hz, 1H), 4.11 (q, J=7.2 Hz, 2H), 3.86 (t, J=6.4 Hz, 2H), 3.12 (s, 2H), 2.85-2.77 (m, 2H), 2.23 (s, 3H), 2.08-1.99 (m, 1H), 1.73-1.67 (m, 3H), 1.63-1.55 (m, 3H), 1.48-1.36 (m, 2H), 1.23-1.18 (m, 5H), 0.84-0.75 (m, 2H)

### Step G. Procedure for Preparation of tert-butyl 6-[8-(1,3-benzothiazol-2-ylcarbamoyl)-3,4-dihydro-1H-isoquinolin-2-yl]-3-[3-[4-[(3S)-1-(2-ethoxy-2-oxo-ethyl)-3-piperidyl]butoxy]-2-methyl-phenyl]pyridine-2-carboxylate

A mixture of ethyl 2-[(3S)-3-[4-(3-bromo-2-methyl-phenoxy)butyl]-1-piperidyl]acetate (0.18 g, 436.51 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (267.38 mg, 436.51 μmol, 1 equiv.), K₃PO₄ (1.5 M, 873.02 μL, 3 equiv.), and [2-(2-aminophenyl)phenyl]palladium(1+); bis(1-adamantyl)-butyl-phosphane; methanesulfonate (31.79 mg, 43.65 μmol, 0.1 equiv.) in dioxane (3 mL) was degassed and purged with N₂ three times, and then the mixture was stirred at 80° C. for 1 hour under N₂ atmosphere. The reaction mixture was concentrated under reduced pressure to give a residue. The residue was purified by flash silica gel chromatography (Eluent of 0˜30% ethyl acetate/petroleum ether) to give tert-butyl 6-[8-(1,3-benzothiazol-2-ylcarbamoyl)-3,4-dihydro-1H-isoquinolin-2-yl]-3-[3-[4-[(3S)-1-(2-ethoxy-2-oxo-ethyl)-3-piperidyl]butoxy]-2-methyl-phenyl]pyridine-2-carboxylate (0.24 g, 228.8 μmol, 52.4% yield, 78% purity) as a yellow oil.

MS (ESI) m/z: 381.7 [(M-56)/2+H]⁺

### Step H. Procedure for Preparation of 2-[(3S)-3-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]butyl]-1-piperidyl]acetic acid

To a solution of tert-butyl 6-[8-(1,3-benzothiazol-2-ylcarbamoyl)-3,4-dihydro-1H-isoquinolin-2-yl]-3-[3-[4-[(3S)-1-(2-ethoxy-2-oxo-ethyl)-3-piperidyl]butoxy]-2-methyl-phenyl]pyridine-2-carboxylate (0.24 g, 228.84 μmol, 78% purity, 1 equiv.) in THE (1 mL) was added LiOH·H₂O (1.5 M, 762.81 μL, 5 equiv.). The mixture was stirred at 20° C. for 1 hour. The mixture was concentrated under reduced pressure, adjusted to pH<7, and filtered to give crude 2-[(3S)-3-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]butyl]-1-piperidyl]acetic acid (0.16 g, 202.5 μmol, 88.5% yield) as a brown solid

¹H NMR (400 MHz, DMSO-d₆) δ=13.32-12.25 (m, 1H), 8.01 (d, J=7.6 Hz, 1H), 7.77 (d, J=8.0 Hz, 1H), 7.60 (d, J=7.6 Hz, 1H), 7.49-7.40 (m, 3H), 7.40-7.31 (m, 2H), 7.14-7.06 (m, 1H), 6.93 (dd, J=8.4, 12.4 Hz, 2H), 6.57 (d, J=7.6 Hz, 1H), 4.98 (s, 2H), 3.97 (t, J=6.0 Hz, 2H), 3.87 (t, J=6.0 Hz, 2H), 3.18 (s, 2H), 3.15 (d, J=2.0 Hz, 2H), 3.03 (t, J=6.0 Hz, 2H), 2.24 (t, J=11.2 Hz, 1H), 1.87 (s, 3H), 1.76-1.56 (m, 7H), 1.50-1.37 (m, 2H), 1.29-1.18 (m, 3H), 1.00 (s, 9H), 0.95-0.88 (m, 1H)

### Step L Procedure for preparation of tert-butyl 6-[8-(1,3-benzothiazol-2-ylcarbamoyl)-3,4-dihydro-1H-isoquinolin-2-yl]-3-[3-[4-[(3S)-1-[2-[[3-(2,6-dioxo-3-piperidyl)-1-methyl-indazol-6-yl]amino]-2-oxo-ethyl]-3-piperidyl]butoxy]-2-methyl-phenyl]pyridine-2-carboxylate

To a solution of 2-[(3S)-3-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]butyl]-1-piperidyl]acetic acid (0.05 g, 63.29 μmol, 1 equiv.) in DMF (0.5 mL) was added HATU (28.88 mg, 75.95 μmol, 1.2 equiv.), TEA (19.21 mg, 189.88 μmol, 26.43 μL, 3 equiv.), and 3-(6-amino-1-methyl-indazol-3-yl)piperidine-2,6-dione (16.35 mg, 63.29 μmol, 1 equiv.). The mixture was stirred at 40° C. for 0.5 hour. The solution was poured into water (2 mL) and then filtered to give a crude tert-butyl 6-[8-(1,3-benzothiazol-2-ylcarbamoyl)-3,4-dihydro-1H-isoquinolin-2-yl]-3-[3-[4-[(3S)-1-[2-[[3-(2,6-dioxo-3-piperidyl)-1-methyl-indazol-6-yl]amino]-2-oxo-ethyl]-3-piperidyl]butoxy]-2-methyl-phenyl]pyridine-2-carboxylate (60 mg, 58.4 μmol, 92.0% yield) as a white solid.

MS (ESI) m/z: 516.1 [M/2+H]⁺

### Step J. Procedure for Preparation of 6-[8-(1,3-benzothiazol-2-ylcarbamoyl)-3,4-dihydro-1H-isoquinolin-2-yl]-3-[3-[4-[(3S)-1-[2-[[3-(2,6-dioxo-3-piperidyl)-1-methyl-indazol-6-yl]amino]-2-oxo-ethyl]-3-piperidyl]butoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[4-[(3S)-1-[2-[[3-(2,6-dioxo-3-piperidyl)-1-methyl-indazol-6-yl]amino]-2-oxo-ethyl]-3-piperidyl]butoxy]-2-methyl-phenyl]pyridine-2-carboxylate (0.06 g, 58.24 μmol, 1 equiv.) in TFA (0.3 mL) was added DCM (0.5 mL). The mixture was stirred at 20° C. for 16 hours. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[4-[(3S)-1-[2-[[3-(2,6-dioxo-3-piperidyl)-1-methyl-indazol-6-yl]amino]-2-oxo-ethyl]-3-piperidyl]butoxy]-2-methyl-phenyl]pyridine-2-carboxylic acid (21.5 mg, 21.4 μmol, 36.8% yield, 96.9% purity) as a yellow solid

MS (ESI) m/z: 975.0 [M+H]⁺

¹H NMR (400 MHz, DMSO-d₆) δ=13.09-12.58 (m, 1H), 10.88 (s, 1H), 10.03-9.69 (m, 1H), 8.07-8.00 (m, 2H), 7.79 (d, J=8.0 Hz, 1H), 7.67-7.57 (m, 2H), 7.50-7.32 (m, 5H), 7.23-7.17 (m, 1H), 7.07 (t, J=8.0 Hz, 1H), 6.99-6.93 (m, 1H), 6.85 (d, J=8.4 Hz, 1H), 6.62 (d, J=7.6 Hz, 1H), 4.98 (s, 2H), 4.32 (dd, J=5.2, 9.6 Hz, 1H), 4.00-3.85 (m, 7H), 3.19-3.12 (m, 2H), 3.03 (t, J=5.6 Hz, 2H), 2.90-2.77 (m, 2H), 2.67-2.59 (m, 2H), 2.38-2.30 (m, 1H), 2.21-2.07 (m, 2H), 1.95-1.83 (m, 4H), 1.78-1.68 (m, 3H), 1.67-1.56 (m, 3H), 1.52-1.42 (m, 2H), 1.32-1.20 (m, 2H), 0.96-0.82 (m, 1H)

### Example 51. Preparation of Compound 205

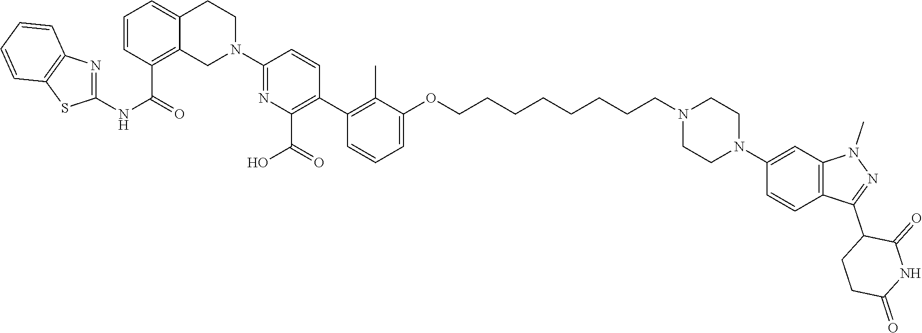 [SMILES: Cc1c(OCCCCCCCCN2CCN(c3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[8-[4-[3-(2,6-dioxo-3-piperidyl)-1-methyl-indazol-6-yl]piperazin-1-yl]octoxy]-2-methyl-phenyl]pyridine-2-carboxylic acid

### Step A. Procedure for Preparation of ethyl 8-(3-bromo-2-methyl-phenoxy)octanoate

A mixture of 3-bromo-2-methyl-phenol (2 g, 10.69 mmol, 1 equiv.), ethyl 8-bromooctanoate (3.22 g, 12.83 mmol, 1.2 equiv.), K₂CO₃ (2.96 g, 21.39 mmol, 2 equiv.) in CH₃CN (20 mL) was degassed and purged with N₂ three times, and then the mixture was stirred at 60° C. for 2 hours under N₂ atmosphere. The reaction mixture was concentrated under reduced pressure to give a residue. The residue was purified by flash silica gel chromatography (Eluent of 0˜8% ethyl acetate/petroleum ether) to give ethyl 8-(3-bromo-2-methyl-phenoxy)octanoate (3.5 g, 9.8 mmol, 91.6% yield) as a colorless.

¹H NMR (400 MHz, DMSO-d₆) δ=7.13-7.06 (m, 2H), 6.95-6.93 (m, 1H), 4.06-4.01 (m, 2H), 3.96 (s, 2H), 2.23 (s, 3H), 1.71 (s, 2H), 1.52 (s, 2H), 1.41 (s, 2H), 1.37-1.21 (m, 6H), 1.18-1.15 (m, 3H).

### Step B. Procedure for Preparation of 8-(3-bromo-2-methyl-phenoxy) octan-1-ol

To a solution of ethyl 8-(3-bromo-2-methyl-phenoxy)octanoate (3.5 g, 9.80 mmol, 1 equiv.) in THE (30 mL) was added LiAlH₄ (557.71 mg, 14.69 mmol, 1.5 equiv.). The mixture was stirred at 0° C. for 2 hours. The reaction mixture was quenched by addition H₂O (0.64 mL), 15% NaOH (0.64 mL), and then extracted with ethyl acetate 30 mL (10 mL×3). The combined organic layers were dried over Na₂SO₄, filtered and concentrated under reduced pressure to give The compound 8-(3-bromo-2-methyl-phenoxy) octan-1-ol (2.8 g, crude) as a colorless oil.

### Step C. Procedure for Preparation of 8-(3-bromo-2-methyl-phenoxy)octanal

DMSO (2.78 g, 35.53 mmol, 2.78 mL, 4 equiv.) in DCM (60 mL) was added a solution of (COCl)₂ (2.25 g, 17.76 mmol, 1.55 mL, 2 equiv.) in DCM (60 mL) at −70° C. under N₂ atmosphere. The mixture was stirred at −70° C. for 1 hour. After which time 8-(3-bromo-2-methyl-phenoxy) octan-1-ol (2.8 g, 8.88 mmol, 1 equiv.) in DCM (60 mL) was added dropwise at −70° C. The solution was stirred for 1 hour at −70° C. Then TEA (5.39 g, 53.29 mmol, 7.42 mL, 6 equiv.) was added into the solution. The solution was stirred at −70° C. for 1.5 hours under N₂ atmosphere. The reaction mixture was concentrated under reduced pressure to give a residue. The residue was purified by flash silica gel chromatography (Eluent of 0˜30% ethyl acetate/petroleum ether) to give 8-(3-bromo-2-methyl-phenoxy)octanal (2.3 g, 7.3 mmol, 82.6% yield) as a white solid.

¹H NMR (400 MHz, DMSO-d₆) δ=9.66 (d, J=1.6 Hz, 1H), 7.16-7.07 (m, 2H), 6.96 (d, J=8.0 Hz, 1H), 3.99-3.94 (m, 2H), 2.51 (d, J=1.6 Hz, 2H), 2.23 (s, 3H), 1.46-1.16 (m, 12H).

### Step D. Procedure for Preparation of tert-butyl 6-[8-(1,3-benzothiazol-2-ylcarbamoyl)-3,4-dihydro-1H-isoquinolin-2-yl]-3-[2-methyl-3-(8-oxooctoxy)phenyl]pyridine-2-carboxylate

A mixture of 8-(3-bromo-2-methyl-phenoxy)octanal (200 mg, 638.51 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (391.12 mg, 638.51 μmol, 1 equiv.), Ad2nBuP Pd G3(cataCXium® A Pd G3) (93.00 mg, 127.70 μmol, 0.2 equiv.), K₂CO₃ (1.5 M, 638.51 μL, 1.5 equiv.) in 1,4-dioxane (6 mL) was degassed and purged with N₂ three times, and then the mixture was stirred at 100° C. for 1 hour under microwave. The reaction mixture was concentrated under reduced pressure to give a residue. The residue was purified by prep-HPLC to give tert-butyl 6-[8-(1,3-benzothiazol-2-ylcarbamoyl)-3,4-dihydro-1H-isoquinolin-2-yl]-3-[2-methyl-3-(8-oxooctoxy)phenyl]pyridine-2-carboxylate (65 mg, 90.4 μmol, 14.1% yield) as a white solid.

MS (ESI) m/z: 719.8 [M+H]⁺

### Step E. Procedure for Preparation of tert-butyl 6-[8-(1,3-benzothiazol-2-ylcarbamoyl)-3,4-dihydro-1H-isoquinolin-2-yl]-3-[3-[8-[4-[3-(2,6-dioxo-3-piperidyl)-1-methyl-indazol-6-yl]piperazin-1-yl]octoxy]-2-methyl-phenyl]pyridine-2-carboxylate

A mixture of tert-butyl 6-[8-(1,3-benzothiazol-2-ylcarbamoyl)-3,4-dihydro-1H-isoquinolin-2-yl]-3-[2-methyl-3-(8-oxooctoxy)phenyl]pyridine-2-carboxylate (65 mg, 90.42 μmol, 1 equiv.), 3-(1-methyl-6-piperazin-1-yl-indazol-3-yl)piperidine-2,6-dione (35.52 mg, 108.50 μmol, 1.2 equiv.), NaBH(OAc)₃ (57.49 mg, 271.25 μmol, 3 equiv.), 4A MS (10 mg, 1.00 equiv.) and AcOH (5.43 mg, 90.42 μmol, 5.18 μL, 1 equiv.) in DCM (2 mL) was degassed and purged with N₂ three times, and then the mixture was stirred at 25° C. for 4 hours under N₂ atmosphere. The reaction mixture was concentrated under reduced pressure to give tert-butyl 6-[8-(1,3-benzothiazol-2-ylcarbamoyl)-3,4-dihydro-1H-isoquinolin-2-yl]-3-[3-[8-[4-[3-(2,6-dioxo-3-piperidyl)-1-methyl-indazol-6-yl]piperazin-1-yl]octoxy]-2-methyl-phenyl]pyridine-2-carboxylate (80 mg, crude) as a white solid.

MS (ESI) m/z: 1030.9 [M+H]⁺

### Step F. Procedure for Preparation of 6-[8-(1,3-benzothiazol-2-ylcarbamoyl)-3,4-dihydro-1H-isoquinolin-2-yl]-3-[3-[8-[4-[3-(2,6-dioxo-3-piperidyl)-1-methyl-indazol-6-yl]piperazin-1-yl]octoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[8-[4-[3-(2,6-dioxo-3-piperidyl)-1-methyl-indazol-6-yl]piperazin-1-yl]octoxy]-2-methyl-phenyl]pyridine-2-carboxylate (80 mg, 77.65 μmol, 1 equiv.) in TFA (1 mL) and DCM (1 mL). The mixture was stirred at 40° C. for 2 hours. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[8-[4-[3-(2,6-dioxo-3-piperidyl)-1-methyl-indazol-6-yl]piperazin-1-yl]octoxy]-2-methyl-phenyl]pyridine-2-carboxylic acid (27.8 mg, 27.3 μmol, 35.2% yield, 95.8% purity) as a white solid.

MS (ESI) m/z: 974.5 [M+H]⁺

¹H NMR (400 MHz, DMSO-d₆) δ=10.85 (s, 1H), 8.14 (s, 1H), 8.04 (d, J=7.6 Hz, 1H), 7.78 (d, J=8.0 Hz, 1H), 7.62 (d, J=6.8 Hz, 1H), 7.50-7.48 (m, 2H), 7.44-7.41 (m, 2H), 7.36 (d, J=8.4 Hz, 2H), 7.11-7.07 (m, 1H), 6.95-6.83 (m, 5H), 6.63 (d, J=7.6 Hz, 1H), 4.98-4.96 (m, 2H), 4.27-4.24 (m, 1H), 3.98-3.95 (m, 2H), 3.90-3.88 (m, 5H), 3.24-3.21 (m, 6H), 3.01-2.98 (m, 2H), 2.62-2.59 (m, 6H), 2.31-2.26 (m, 1H), 2.19-2.13 (m, 1H), 1.90 (s, 3H), 1.77-1.73 (m, 2H), 1.48-1.44 (m, 4H), 1.36-1.29 (m, 6H).

### Example 52. Preparation of Compound 207a

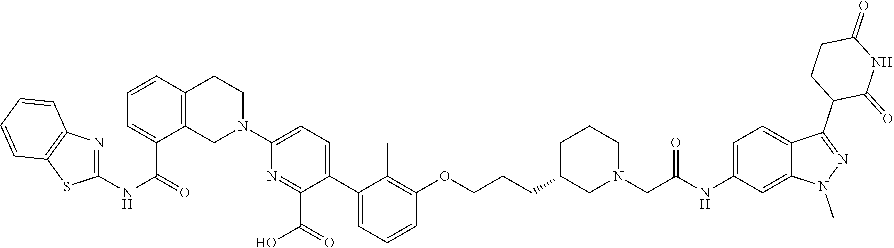 [SMILES: Cc1c(OCCC[C@@H]2CCCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-((3S)-1-(2-((3-(2,6-dioxo piperidin-3-yl)-1-methyl-1H-indazol-6-yl)amino)-2-oxoethyl)piperidin-3-yl)propoxy)-2-methylphenyl) picolinic acid

### Step A. Procedure for Preparation of (R)-tert-butyl 3-formylpiperidine-1-carboxylate

To a solution of tert-butyl (3R)-3-(hydroxymethyl) piperidine-1-carboxylate (10 g, 46.45 mmol, 1 equiv.) in DCM (10 mL) was added DMP (19.70 g, 46.45 mmol, 14.38 mL, 1 equiv.) at 0° C. The reaction mixture was degassed and purged with N₂ three times, and then the mixture was stirred at 0° C. for 2 hours under N₂ atmosphere. The reaction mixture was diluted with Na₂S203 (10 mL) and extracted with DCM (20 mL×3). The combined organic layers were washed with brine (10 mL×3), dried over anhydrous sodium sulfate, filtered, and concentrated under reduced pressure to give (R)-tert-butyl 3-formylpiperidine-1-carboxylate (9.29 g, crude) as a colorless oil.

¹H NMR (400 MHz, DMSO-d₆) δ=9.59 (s, 1H), 3.82-3.65 (m, 1H), 3.55-3.35 (m, 2H), 3.12-3.02 (m, 1H), 2.49-2.41 (m, 1H), 1.89-1.81 (m, 1H), 1.64-1.51 (m, 2H), 1.39 (s, 10H)

### Step B. Procedure for Preparation of (S,E)-tert-butyl 3-(3-ethoxy-3-oxoprop-1-en-1-yl)piperidine-1-carboxylate

To a solution of ethyl 2-(diethoxyphosphoryl)acetate (19.53 g, 87.12 mmol, 17.28 mL, 2 equiv.) in THE (100 mL) was added NaH (2.30 g, 57.50 mmol, 60% purity, 1.32 equiv.) in portions under N₂ at 0° C. The mixture was stirred at 0° C. for 1 hour. (R)-tert-butyl 3-formylpiperidine-1-carboxylate (9.29 g, 43.56 mmol, 1 equiv.) was added to the mixture, and then the mixture was stirred at 25° C. for 16 hours. The reaction mixture was quenched by addition NH₄Cl (50 mL) at 0° C., and extracted with ethyl acetate (150 mL×3). The combined organic layers were washed with brine 100 mL (100 mL×1), dried over Na₂SO₄, filtered, and concentrated under reduced pressure to give a residue. The residue was purified by column chromatography (SiO₂, petroleum ether/ethyl acetate 1/0 to 20/1) to give (S,E)-tert-butyl 3-(3-ethoxy-3-oxoprop-1-en-1-yl)piperidine-1-carboxylate (6.7 g, 22.1 mmol, 50.8% yield, 93.7% purity) as a colorless oil.

¹H NMR (400 MHz, CDCl₃) δ=6.85 (dd, J=6.8, 16.0 Hz, 1H), 5.87 (d, J=16.0 Hz, 1H), 4.20 (q, J=7.2 Hz, 2H), 3.95-3.86 (m, 1H), 2.96-2.61 (m, 2H), 2.46-2.28 (m, 1H), 1.93-1.85 (m, 1H), 1.74-1.57 (m, 2H), 1.56-1.49 (m, 1H), 1.47 (s, 9H), 1.44-1.34 (m, 1H), 1.30 (t, J=7.2 Hz, 3H)

### Step C. Procedure for Preparation of (S)-tert-butyl 3-(3-ethoxy-3-oxopropyl)piperidine-1-carboxylate

To a solution of (S,E)-tert-butyl 3-(3-ethoxy-3-oxoprop-1-en-1-yl)piperidine-1-carboxylate (2 g, 7.06 mmol, 1 equiv.) in EtOH (20 mL) was added PtO₂ (160.27 mg, 705.81 μmol, 0.1 equiv.) under N₂. The suspension was degassed under vacuum and purged with H₂ three times. The mixture was stirred under H₂ (15 psi) at 25° C. for 24 hours. The reaction mixture was filtered and concentrated under reduced pressure to give a residue. The residue was purified by flash silica gel chromatography (Eluent of 0˜10% Ethylacetate/petroleum ether) to give (S)-tert-butyl 3-(3-ethoxy-3-oxopropyl)piperidine-1-carboxylate (2.8 g, crude) as a colorless oil.

¹H NMR (400 MHz, CDCl₃) δ=4.18-4.09 (m, 2H), 3.96-3.88 (m, 1H), 2.85-2.74 (m, 1H), 2.51 (t, J=11.6 Hz, 1H), 2.34 (t, J=7.6 Hz, 2H), 1.87-1.78 (m, 1H), 1.67-1.59 (m, 2H), 1.59-1.49 (m, 2H), 1.46 (s, 9H), 1.45-1.38 (m, 1H), 1.26 (t, J=7.1 Hz, 3H), 1.15-1.04 (m, 1H).

SFC: Column: Chiralpak IC-3 50×4.6 mm ID, 3 um, Mobile phase: Phase A for CO₂, and Phase B for MeOH (0.05% DEA); Gradient elution: B in A from 5% to 40% Flow rate: 3 mL/min; Detector: PDA; Column Temp: 35 C; Back Pressure: 100 Bar

### Step D. Procedure for Preparation of (S)-tert-butyl 3-(3-hydroxypropyl)piperidine-1-carboxylate

To a solution of (S)-tert-butyl 3-(3-ethoxy-3-oxopropyl)piperidine-1-carboxylate (2.8 g, 9.81 mmol, 1 equiv.) in THE (30 mL) was added LiAlH₄ (372.39 mg, 9.81 mmol, 1 equiv.) dropwise at 0° C. under N₂. The reaction mixture was stirred under N₂ at 0° C. for 2 hours. The reaction mixture was quenched with Na₂SO₄·10 H₂O (100 mg) and slowly warmed to 20° C. The mixture was filtered and the filter cake was washed with DCM (50 mL). Then the organic layers were concentrated under reduced pressure to give a residue. The residue was purified by flash silica gel chromatography (Eluent of 0˜10% Ethylacetate/petroleum ether) to give (S)-tert-butyl 3-(3-hydroxypropyl)piperidine-1-carboxylate (2.2 g, 7.5 mmol, 76.8% yield, 83.4% purity) as a colorless oil.

¹H NMR (400 MHz, DMSO-d₆) δ=4.51-4.23 (m, 1H), 3.86-3.63 (m, 2H), 3.46-3.34 (m, 3H), 2.80-2.71 (m, 1H), 1.78-1.70 (m, 1H), 1.60-1.52 (m, 1H), 1.46-1.40 (m, 2H), 1.38 (s, 9H), 1.35-1.24 (m, 2H), 1.22-1.12 (m, 2H), 1.08-0.99 (m, 1H)

### Step E. Procedure for Preparation of (S)-tert-butyl 3-(3-(3-bromo-2-methylphenoxy)propyl)piperidine-1-carboxylate

To a solution of (S)-tert-butyl 3-(3-hydroxypropyl)piperidine-1-carboxylate (2.2 g, 9.04 mmol, 1 equiv.) in toluene (25 mL) was added 3-bromo-2-methylphenol (1.69 g, 9.04 mmol, 1 equiv.) and 2-(tributyl-λ⁵-phosphanylidene) acetonitrile (2.62 g, 10.85 mmol, 1.2 equiv.) at 25° C. The reaction mixture was degassed and purged with N₂ three times, and then the mixture was stirred at 120° C. for 16 hours under N₂ atmosphere. The mixture was filtered, and the filter cake was washed with DCM (50 mL). Then the organic layers were concentrated under reduced pressure to give a residue. The residue was purified by column chromatography (SiO₂, petroleum ether/ethyl acetate 1/0 to 20/1) to give (S)-tert-butyl 3-(3-(3-bromo-2-methylphenoxy)propyl)piperidine-1-carboxylate (3.1 g, 6.1 mmol, 68.0% yield, 80% purity) as a colorless oil.

¹H NMR (400 MHz, CDCl₃) δ=7.14 (d, J=8.0 Hz, 1H), 6.99 (t, J=8.0 Hz, 1H), 6.75 (d, J=8.0 Hz, 1H), 4.03-3.89 (m, 4H), 2.83-2.75 (m, 1H), 2.55-2.45 (m, 1H), 2.31 (s, 3H), 1.92-1.80 (m, 3H), 1.70-1.62 (m, 1H), 1.55-1.48 (m, 1H), 1.47 (s, 9H), 1.45-1.32 (m, 3H), 1.17-1.05 (m, 1H)

### Step F. Procedure for Preparation of (S)-3-(3-(3-bromo-2-methylphenoxy)propyl)piperidine

To a solution of (S)-tert-butyl 3-(3-(3-bromo-2-methylphenoxy)propyl)piperidine-1-carboxylate (3.17 g, 6.15 mmol, 80% purity, 1 equiv.) in DCM (10 mL) was added HCl/dioxane (4 M, 15 mL, 9.76 equiv.) at 25° C. The reaction mixture was stirred at 25° C. for 1 hour. The reaction mixture was filtered to give (S)-3-(3-(3-bromo-2-methylphenoxy)propyl)piperidine (2.24 g, crude) as a yellow solid.

¹H NMR (400 MHz, CD₃OD) δ=7.14-7.10 (m, 1H), 7.03 (t, J=8.0 Hz, 1H), 6.89 (d, J=8.0 Hz, 1H), 4.00 (t, J=6.4 Hz, 2H), 3.41-3.32 (m, 2H), 2.95-2.86 (m, 1H), 2.66 (t, J=12.0 Hz, 1H), 2.29 (s, 3H), 2.04-1.67 (m, 6H), 1.57-1.46 (m, 2H), 1.33-1.19 (m, 1H)

### Step G. Procedure for Preparation of (S)-ethyl 2-(3-(3-(3-bromo-2-methylphenoxy)propyl)piperidin-1-yl)acetate

To a solution of (S)-3-(3-(3-bromo-2-methylphenoxy)propyl)piperidine (2.2 g, 6.31 mmol, 1 equiv., HCl) in CH₃CN (20 mL) was added ethyl 2-bromoacetate (948.26 mg, 5.68 mmol, 627.98 μL, 0.9 equiv.) and K₂CO₃ (2.62 g, 18.93 mmol, 3 equiv.) at 25° C. The reaction mixture was stirred at 60° C. for 2 hours. The reaction mixture was filtered and concentrated under reduced pressure to give a residue. The residue was purified by flash silica gel chromatography (Eluent of 0˜2% MeOH/DCM) to give (S)-ethyl 2-(3-(3-(3-bromo-2-methylphenoxy)propyl)piperidin-1-yl)acetate (1.9 g, 4.7 mmol, 75.6% yield) as a colorless oil.

¹H NMR (400 MHz, CDCl₃-d) δ=7.14 (d, J=8.0 Hz, 1H), 6.99 (t, J=8.0 Hz, 1H), 6.75 (d, J=8.0 Hz, 1H), 4.19 (q, J=7.2 Hz, 2H), 3.93 (t, J=6.4 Hz, 2H), 3.21 (s, 2H), 2.95-2.86 (m, 2H), 2.31 (s, 3H), 2.18-2.08 (m, 1H), 1.90-1.76 (m, 4H), 1.75-1.65 (m, 3H), 1.44-1.32 (m, 2H), 1.31-1.26 (m, 3H), 0.98-0.84 (m, 1H)

### Step H. Procedure for Preparation of (S)-tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)piperidin-3-yl)propoxy)-2-methylphenyl)picolinate

tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (600 mg, 979.52 μmol, 1 equiv.), (S)-ethyl 2-(3-(3-(3-bromo-2-methylphenoxy)propyl)piperidin-1-yl)acetate (390.18 mg, 979.52 μmol, 1 equiv.), KF (1.5 M, 1.96 mL, 3 equiv.) and [2-(2-aminophenyl) phenyl]palladium (1+); bis (1-adamantyl)-butyl-phosphane; methanesulfonate (71.34 mg, 97.95 μmol, 0.1 equiv.) were taken up into a microwave tube in dioxane (6 mL). The sealed tube was heated at 100° C. for 60 minutes under microwave. The reaction mixture was filtered and concentrated under reduced pressure to give a residue. The residue was purified by flash silica gel chromatography (Eluent of 0˜50% ethyl acetate/petroleum ether) to give (S)-tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)piperidin-3-yl)propoxy)-2-methylphenyl)picolinate (476 mg, 558.2 μmol, 57.0% yield, 94.3% purity) as a white solid.

MS (ESI) m/z: 804.8 [M+H]⁺.

### Step I. Procedure for preparation of (S)-2-(3-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)acetic acid

To a solution of (S)-tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)piperidin-3-yl)propoxy)-2-methylphenyl)picolinate (476 mg, 592.04 μmol, 1 equiv.) in THE (5 mL) and H₂O (0.5 mL) was added LiOH·H₂O (74.53 mg, 1.78 mmol, 3 equiv.) at 25° C. The reaction mixture was stirred at 25° C. for 16 hours. The reaction mixture was concentrated under reduced pressure to give (S)-2-(3-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)acetic acid (580 mg, crude) as a yellow solid.

MS (ESI) m/z: 776.8 [M+H]⁺.

### Step J. Procedure for Preparation of tert-butyl 6-(8-(benzo[d]thiazol-2-ylcarbamoyl)-3,4-dihydroisoquinolin-2(1H)-yl)-3-(3-(3-((3S)-1-(2-((3-(2,6-dioxopiperidin-3-yl)-1-methyl-1H-indazol-6-yl)amino)-2-oxoethyl)piperidin-3-yl)propoxy)-2-methylphenyl)picolinate

To a solution of (S)-2-(3-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)acetic acid (150 mg, 193.31 μmol, 1 equiv.) in DMF (1.5 mL) was added 3-(6-amino-1-methyl-1H-indazol-3-yl)piperidine-2,6-dione (49.93 mg, 193.31 μmol, 1 equiv.), HATU (80.85 mg, 212.64 mol, 1.1 equiv.), and DIPEA (74.95 mg, 579.93 μmol, 101.01 μL, 3 equiv.) at 25° C. The reaction mixture was stirred at 25° C. for 16 hours. The mixture was triturated with water and filtered to give tert-butyl 6-(8-(benzo[d]thiazol-2-ylcarbamoyl)-3,4-dihydroisoquinolin-2(1H)-yl)-3-(3-(3-((3S)-1-(2-((3-(2,6-dioxopiperidin-3-yl)-1-methyl-1H-indazol-6-yl)amino)-2-oxoethyl)piperidin-3-yl)propoxy)-2-methylphenyl)picolinate (140 mg, 112.3 μmol, 58.0% yield, 81.5% purity) as a yellow solid.

MS (ESI) m/z: 1016.8 [M+H]⁺.

### Step K. Procedure for Preparation of 6-(8-(benzo[d]thiazol-2-ylcarbamoyl)-3,4-dihydroisoquinolin-2(1H)-yl)-3-(3-(3-((3S)-1-(2-((3-(2,6-dioxo piperidin-3-yl)-1-methyl-11H-indazol-6-yl)amino)-2-oxoethyl)piperidin-3-yl)propoxy)-2-methylphenyl) picolinic acid

A solution of tert-butyl 6-(8-(benzo[d]thiazol-2-ylcarbamoyl)-3,4-dihydroisoquinolin-2(1H)-yl)-3-(3-(3-((3S)-1-(2-((3-(2,6-dioxopiperidin-3-yl)-1-methyl-1H-indazol-6-yl)amino)-2-oxoethyl)piperidin-3-yl)propoxy)-2-methylphenyl)picolinate (140 mg, 112.28 μmol, 81.5% purity, 1 equiv.) in DCM (1 mL) and TFA (1 mL) was stirred at 25° C. for 16 hours. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(3-((3S)-1-(2-((3-(2,6-dioxo piperidin-3-yl)-1-methyl-1H-indazol-6-yl)amino)-2-oxoethyl)piperidin-3-yl)propoxy)-2-methylphenyl) picolinic acid (41.0 mg, 41.5 μmol, 37.0% yield, 97.1% purity) as a white solid.

MS (ESI) m/z: 960.7 [M+H]⁺.

¹H NMR (400 MHz, DMSO-d₆) δ=10.87 (s, 1H), 9.85 (s, 1H), 8.06-8.00 (m, 2H), 7.78 (d, J=8.0 Hz, 1H), 7.65-7.58 (m, 2H), 7.49-7.32 (m, 5H), 7.20 (d, J=8.4 Hz, 1H), 7.09-7.04 (m, 1H), 6.94 (d, J=8.8 Hz, 1H), 6.85 (d, J=8.4 Hz, 1H), 6.62 (d, J=7.6 Hz, 1H), 4.98 (s, 2H), 4.31 (dd, J=5.2, 9.6 Hz, 1H), 3.97-3.91 (m, 3H), 3.90 (s, 4H), 3.20-3.06 (m, 4H), 3.02 (t, J=5.6 Hz, 2H), 2.90-2.77 (m, 2H), 2.69-2.59 (m, 2H), 2.38-2.28 (m, 1H), 2.20-2.09 (m, 2H), 1.90 (s, 1H), 1.88 (s, 3H), 1.81-1.72 (m, 3H), 1.66-1.58 (m, 2H), 1.37 (d, J=6.4 Hz, 2H)

### Example 53. Preparation of Compound 208

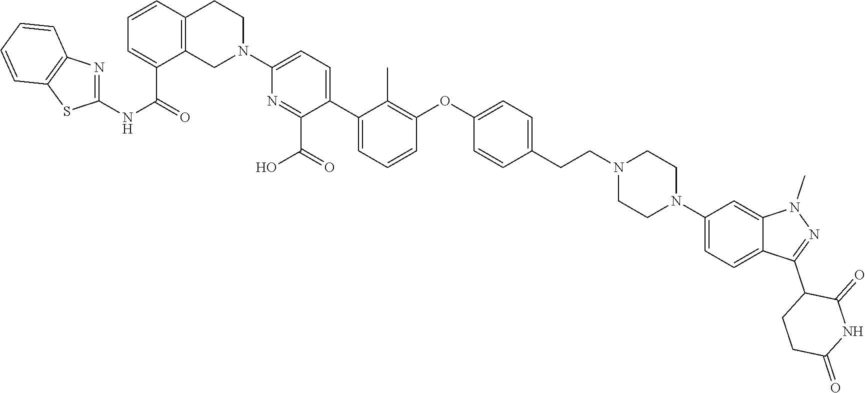 [SMILES: Cc1c(Oc2ccc(CCN3CCN(c4ccc5c(C6CCC(=O)NC6=O)nn(C)c5c4)CC3)cc2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[4-[2-[4-[3-(2,6-dioxo-3-piperidyl)-1-methyl-indazol-6-yl]piperazin-1-yl]ethyl]phenoxy]-2-methyl-phenyl]pyridine-2-carboxylic acid

### Step A. Procedure for Preparation of methyl 2-[4-(3-bromo-2-methyl-phenoxy)phenyl]acetate

A mixture of methyl 2-(4-hydroxyphenyl)acetate (1.7 g, 10.23 mmol, 1 equiv.), 1,3-dibromo-2-methyl-benzene (7.67 g, 30.69 mmol, 3 equiv.), CuI (974.18 mg, 5.12 mmol, 0.5 equiv.), cesium carbonate (4.00 g, 12.28 mmol, 1.2 equiv.), and 2,2,6,6-tetramethylheptane-3,5-dione (471.30 mg, 2.56 mmol, 526.59 μL, 0.25 equiv.) in NMP (20 mL) was stirred at 120° C. for 16 hours under N₂ atmosphere. The reaction mixture was concentrated under reduced pressure to give a residue. The residue was purified by flash silica gel chromatography (Eluent of 0˜3% ethyl acetate/petroleum ether) to give methyl 2-[4-(3-bromo-2-methyl-phenoxy)phenyl]acetate (3 g, 8.9 mmol, 87.4% yield) as a yellow oil.

¹H NMR (400 MHz, DMSO-d₆) δ=7.44 (d, J=8.0 Hz, 1H), 7.26 (d, J=8.8 Hz, 2H), 7.17 (t, J=8.0 Hz, 1H), 6.91 (d, J=8.4 Hz, 1H), 6.88 (d, J=8.4 Hz, 2H), 3.65 (s, 2H), 3.61 (s, 3H), 2.26 (s, 3H)

### Step B. Procedure for Preparation of 2-[4-(3-bromo-2-methyl-phenoxy)phenyl]ethanol

To a solution of methyl 2-[4-(3-bromo-2-methyl-phenoxy)phenyl]acetate (1 g, 2.98 mmol, 1 equiv.) in THF (10 mL) was purged with N₂ three times and added in portions LiAlH₄ (90.59 mg, 2.39 mmol, 0.8 equiv.) under N₂ at 0° C. The mixture was stirred under N₂ at 0° C. for 0.5 hour. The reaction mixture was quenched with Na₂SO₄·10 H₂O (100 mg) and slowly warmed to 20° C. The mixture was filtered and washed with DCM (10 mL). Then the organic layers were concentrated under reduced pressure to give a residue. The residue was purified by flash silica gel chromatography (Eluent of 0˜30% ethyl acetate/petroleum ether) to give 2-[4-(3-bromo-2-methyl-phenoxy)phenyl]ethanol (760 mg, 2.4 mmol, 82.9% yield) as a white oil.

¹H NMR (400 MHz, DMSO-d₆) δ=7.41 (dd, J=0.8, 8.0 Hz, 1H), 7.22-7.19 (m, 2H), 7.17-7.12 (m, 1H), 6.87-6.82 (m, 3H), 4.64-4.61 (m, 1H), 3.61-3.57 (m, 2H), 2.72-2.67 (m, 2H), 2.26 (s, 3H)

### Step C. Procedure for Preparation of 2-[4-(3-bromo-2-methyl-phenoxy)phenyl]acetaldehyde

To a solution of 2-[4-(3-bromo-2-methyl-phenoxy)phenyl]ethanol (760 mg, 2.47 mmol, 1 equiv.) in DCM (7.6 mL) was added DMP (1.26 g, 2.97 mmol, 919.16 μL, 1.2 equiv.) in an ice bath. The mixture was stirred at 0° C. for 2 hours. The reaction mixture was quenched by addition Na₂S₂O₃ (5 mL) at 0° C. and extracted with DCM (5 mL), filtered, and concentrated under reduced pressure to give 2-[4-(3-bromo-2-methyl-phenoxy)phenyl]acetaldehyde (700 mg, crude) as a yellow solid.

¹H NMR (400 MHz, DMSO-d₆) δ=9.69-9.66 (m, 1H), 7.98 (dd, J=1.2, 8.0 Hz, 1H), 7.70 (dd, J=1.6, 7.6 Hz, 1H), 7.44 (dd, J=0.8, 8.0 Hz, 1H), 7.25-7.22 (m, 2H), 6.92-6.89 (m, 2H), 3.77-3.71 (m, 2H), 1.91 (s, 3H)

### Step D. Procedure for Preparation of 2-[[4-(3-bromo-2-methyl-phenoxy)phenyl]methyl]-1,3-dioxolane

To a solution of 2-[4-(3-bromo-2-methyl-phenoxy)phenyl]acetaldehyde (600 mg, 1.97 mmol, 1 equiv.) and ethylene glycol (1.85 g, 29.81 mmol, 1.67 mL, 15.16 equiv.) in toluene (50 mL) was added TsOH (67.71 mg, 393.23 μmol, 0.2 equiv.). The mixture was stirred at 140° C. for 16 hours. The reaction mixture was concentrated under reduced pressure to give a residue. The residue was purified by flash silica gel chromatography (Eluent of 0˜10% ethyl acetate/petroleum ether) to give 2-[[4-(3-bromo-2-methyl-phenoxy)phenyl]methyl]-1,3-dioxolane (680 mg, crude) as a yellow oil.

¹H NMR (400 MHz, DMSO-d₆) δ=7.42 (dd, J=0.8, 8.0 Hz, 1H), 7.26-7.22 (m, 2H), 7.15 (t, J=8.0 Hz, 1H), 6.90-6.85 (m, 2H), 6.85-6.83 (m, 1H), 4.96-4.92 (m, 1H), 3.89-3.85 (m, 2H), 3.77-3.73 (m, 2H), 2.84 (d, J=5.2 Hz, 2H), 2.26 (s, 3H)

### Step E. Procedure for Preparation of tert-butyl 6-[8-(1,3-benzothiazol-2-ylcarbamoyl)-3,4-dihydro-1H-isoquinolin-2-yl]-3-[3-[4-(1,3-dioxolan-2-ylmethyl)phenoxy]-2-methyl-phenyl]pyridine-2-carboxylate

2-[[4-(3-bromo-2-methyl-phenoxy)phenyl]methyl]-1,3-dioxolane (680 mg, 1.95 mmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (1.19 g, 1.95 mmol, 1 equiv.), KF (1.5 M, 3.89 mL, 3 equiv.), and [2-(2-aminophenyl)phenyl]palladium(1+); bis(1-adamantyl)-butyl-phosphane; methanesulfonate (283.62 mg, 389.44 μmol, 0.2 equiv.) were taken up into a microwave tube in dioxane (6.8 mL). The sealed tube was heated at 100° C. for 1 hour under microwave. The reaction mixture was concentrated under reduced pressure to give a residue. The residue was purified by flash silica gel chromatography (Eluent of 0-20% ethyl acetate/petroleum ether) to give tert-butyl 6-[8-(1,3-benzothiazol-2-ylcarbamoyl)-3,4-dihydro-1H-isoquinolin-2-yl]-3-[3-[4-(1,3-dioxolan-2-ylmethyl)phenoxy]-2-methyl-phenyl]pyridine-2-carboxylate (650 mg, 800.8 μmol, 41.1% yield, 93% purity) as a white solid.

MS (ESI) m/z: 755.5[M+H]⁺.

¹H NMR (400 MHz, DMSO-d₆) δ=12.86 (s, 1H), 8.02 (d, J=8.0 Hz, 1H), 7.78 (d, J=8.0 Hz, 1H), 7.60 (d, J=7.6 Hz, 1H), 7.52 (d, J=8.8 Hz, 1H), 7.49-7.43 (m, 2H), 7.36 (d, J=7.6, 12.4 Hz, 2H), 7.24-7.16 (m, 3H), 6.97 (d, J=8.8 Hz, 1H), 6.89-6.79 (m, 4H), 4.99 (s, 2H), 4.93 (t, J=5.2 Hz, 1H), 3.90-3.84 (m, 4H), 3.80-3.72 (m, 2H), 3.04 (t, J=6.0 Hz, 2H), 2.83 (d, J=4.8 Hz, 2H), 1.90 (s, 3H), 1.06 (d, J=2.0 Hz, 9H)

### Step F. Procedure for Preparation of 6-[8-(1,3-benzothiazol-2-ylcarbamoyl)-3,4-dihydro-1H-isoquinolin-2-yl]-3-[2-methyl-3-[4-(2-oxoethyl)phenoxy]phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[4-(1,3-dioxolan-2-ylmethyl)phenoxy]-2-methyl-phenyl]pyridine-2-carboxylate (200 mg, 264.94 μmol, 1 equiv.) in HCOOH (3 mL) was stirred at 100° C. for 2 hours. The reaction mixture was concentrated under reduced pressure to give 6-[8-(1,3-benzothiazol-2-ylcarbamoyl)-3,4-dihydro-1H-isoquinolin-2-yl]-3-[2-methyl-3-[4-(2-oxoethyl)phenoxy]phenyl]pyridine-2-carboxylic acid (180 mg, 237.5 μmol, 89.6% yield, 86.4% purity) as a white solid.

MS (ESI) m/z: 655.5[M+H]⁺.

### Step G. Procedure for Preparation of 6-[8-(1,3-benzothiazol-2-ylcarbamoyl)-3,4-dihydro-1H-isoquinolin-2-yl]-3-[3-[4-[2-[4-[3-(2,6-dioxo-3-piperidyl)-1-methyl-indazol-6-yl]piperazin-1-yl]ethyl]phenoxy]-2-methyl-phenyl]pyridine-2-carboxylic acid

To a solution of 6-[8-(1,3-benzothiazol-2-ylcarbamoyl)-3,4-dihydro-1H-isoquinolin-2-yl]-3-[2-methyl-3-[4-(2-oxoethyl)phenoxy]phenyl]pyridine-2-carboxylic acid (50 mg, 76.37 μmol, 1 equiv.) and 3-(1-methyl-6-piperazin-1-yl-indazol-3-yl)piperidine-2,6-dione (75.00 mg, 229.10 μmol, 3 equiv.) in EtOH (1 mL) and THE (1 mL) was added AcOH (458.58 μg, 7.64 μmol, 4.37e-1 μL, 0.1 equiv.). The mixture was stirred at 25° C. for 15.5 hours. Then was added NaBH₃CN (14.40 mg, 229.10 μmol, 3 equiv.). The mixture was stirred at 25° C. for 0.5 hours. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[4-[2-[4-[3-(2,6-dioxo-3-piperidyl)-1-methyl-indazol-6-yl]piperazin-1-yl]ethyl]phenoxy]-2-methyl-phenyl]pyridine-2-carboxylic acid (14.4 mg, 14.0 μmol, 36.6% yield, 93.7% purity) as a yellow solid.

MS (ESI) m/z: 967.1[M+H]⁺.

¹H NMR (400 MHz, DMSO-d₆) δ=10.85 (s, 1H), 8.03 (d, J=7.6 Hz, 1H), 7.79 (d, J=8.0 Hz, 1H), 7.62 (d, J=7.6 Hz, 1H), 7.53-7.48 (m, 2H), 7.48-7.43 (m, 2H), 7.39-7.33 (m, 2H), 7.22 (d, J=8.4 Hz, 2H), 7.17 (t, J=7.6 Hz, 1H), 7.00 (d, J=8.8 Hz, 1H), 6.94-6.91 (m, 1H), 6.89 (d, J=7.2 Hz, 2H), 6.85-6.81 (m, 3H), 5.00 (s, 2H), 4.26 (dd, J=5.2, 9.2 Hz, 1H), 3.92 (t, J=6.0 Hz, 2H), 3.89 (s, 3H), 3.24 (s, 4H), 3.03 (t, J=5.6 Hz, 2H), 2.79-2.74 (m, 2H), 2.66 (s, 4H), 2.63-2.58 (m, 4H), 2.35-2.25 (m, 1H), 2.20-2.11 (m, 1H), 1.90 (s, 3H)

### Example 54. Preparation of Compound 219a

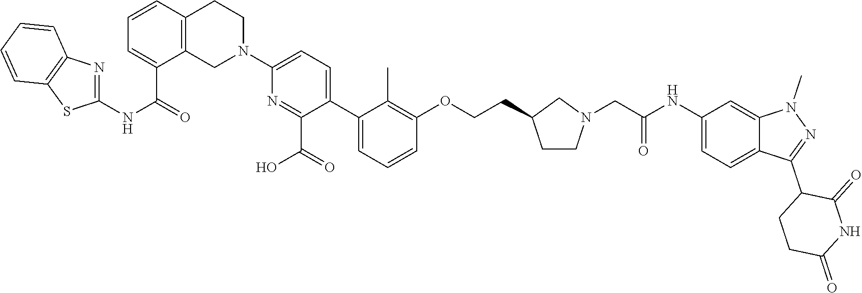 [SMILES: Cc1c(OCC[C@@H]2CCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[2-[(3S)-1-[2-[[3-(2,6-dioxo-3-piperidyl)-1-methyl-indazol-6-yl]amino]-2-oxo-ethyl]pyrrolidin-3-yl]ethoxy]-2-methyl-phenyl]pyridine-2-carboxylic acid

### Step A. Procedure for Preparation of tert-butyl (3S)-3-[2-(3-bromo-2-methyl-phenoxy)ethyl]pyrrolidine-1-carboxylate

A mixture of 3-bromo-2-methyl-phenol (1.30 g, 6.97 mmol, 1 equiv.), tert-butyl (3S)-3-(2-hydroxyethyl)pyrrolidine-1-carboxylate (1.5 g, 6.97 mmol, 1 equiv.), 2-(tributyl-λ⁵-phosphanylidene)acetonitrile (2.52 g, 10.45 mmol, 1.5 equiv.) in toluene (20 mL) was degassed and purged with N₂ three times, and then the mixture was stirred at 120° C. for 6 hours under N₂ atmosphere. The reaction mixture was concentrated under reduced pressure to give a residue. The residue was purified by flash silica gel chromatography (Eluent of 0˜10% ethyl acetate/petroleum ether) to give tert-butyl (3S)-3-[2-(3-bromo-2-methyl-phenoxy)ethyl]pyrrolidine-1-carboxylate (2.3 g, 6.1 mmol, 88.8% yield) as a yellow oil.

### Step B. Procedure for Preparation of (3S)-3-[2-(3-bromo-2-methyl-phenoxy)ethyl]pyrrolidine

A mixture of tert-butyl (3S)-3-[2-(3-bromo-2-methyl-phenoxy)ethyl]pyrrolidine-1-carboxylate (2.38 g, 6.19 mmol, 1 equiv.) in HCl/EtOAc (10 mL) was degassed and purged with N₂ three times, and then the mixture was stirred at 25° C. for 2 hours under N₂ atmosphere. The reaction mixture was concentrated under reduced pressure to give a residue. The compound (3S)-3-[2-(3-bromo-2-methyl-phenoxy)ethyl]pyrrolidine (1.6 g, 4.9 mmol, 80.5% yield) was obtained as a white solid.

### Step C. Procedure for Preparation of ethyl 2-[(3S)-3-[2-(3-bromo-2-methyl-phenoxy)ethyl]pyrrolidin-1-yl]acetate

A mixture of (3S)-3-[2-(3-bromo-2-methyl-phenoxy)ethyl]pyrrolidine (1.6 g, 5.63 mmol, 1 equiv.), ethyl 2-bromoacetate (940.22 mg, 5.63 mmol, 622.66 μL, 1 equiv.), K₂CO₃ (3.89 g, 28.15 mmol, 5 equiv.) in CH₃CN (10 mL) was degassed and purged with N₂ three times, and then the mixture was stirred at 60° C. for 2 hours under N₂ atmosphere. The reaction mixture was concentrated under reduced pressure to give a residue. The residue was purified by flash silica gel chromatography (Eluent of 0˜23% ethyl acetate/petroleum ether) to give ethyl 2-[(3S)-3-[2-(3-bromo-2-methyl-phenoxy)ethyl]pyrrolidin-1-yl]acetate (1.1 g, 2.9 mmol, 51.6% yield, 97.9% purity) as a yellow oil.

MS (ESI) m/z: 372.3 [M+2]⁺

### Step D. Procedure for Preparation of tert-butyl 6-[8-(1,3-benzothiazol-2-ylcarbamoyl)-3,4-dihydro-1H-isoquinolin-2-yl]-3-[3-[2-[(3S)-1-(2-ethoxy-2-oxo-ethyl)pyrrolidin-3-yl]ethoxy]-2-methyl-phenyl]pyridine-2-carboxylate

A mixture of ethyl 2-[(3S)-3-[2-(3-bromo-2-methyl-phenoxy)ethyl]pyrrolidin-1-yl]acetate (300 mg, 810.20 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (496.28 mg, 810.20 μmol, 1 equiv.), K₂CO₃ (1.5 M, 810.20 μL, 1.5 equiv.), Ad₂nBuP Pd G₃ (118.01 mg, 162.04 μmol, 0.2 equiv.) in dioxane (5 mL) was degassed and purged with N₂ three times, and then the mixture was stirred at 100° C. for 1 hour in microwave. The reaction mixture was concentrated under reduced pressure to give a residue. The residue was purified by flash silica gel chromatography (Eluent of 0˜100% ethyl acetate/petroleum ether) to give tert-butyl 6-[8-(1,3-benzothiazol-2-ylcarbamoyl)-3,4-dihydro-1H-isoquinolin-2-yl]-3-[3-[2-[(3S)-1-(2-ethoxy-2-oxo-ethyl)pyrrolidin-3-yl]ethoxy]-2-methyl-phenyl]pyridine-2-carboxylate (430 mg, 554.1 μmol, 68.4% yield) as a yellow oil.

MS (ESI) m/z: 776.9 [M+H]⁺.

### Step E. Procedure for Preparation of 2-[(3S)-3-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]ethyl]pyrrolidin-1-yl]acetic acid

A mixture of tert-butyl 6-[8-(1,3-benzothiazol-2-ylcarbamoyl)-3,4-dihydro-1H-isoquinolin-2-yl]-3-[3-[2-[(3S)-1-(2-ethoxy-2-oxo-ethyl)pyrrolidin-3-yl]ethoxy]-2-methyl-phenyl]pyridine-2-carboxylate (105 mg, 135.32 μmol, 1 equiv.), LiOH (1 M, 405.95 μL, 3 equiv.) in THE (1 mL) was degassed and purged with N₂ three times, and then the mixture was stirred at 25° C. for 2 hours under N₂ atmosphere. The reaction mixture was concentrated under reduced pressure to give a residue. The residue was taken up in HCl 0.5 mL and filtered. The solid was dissolved in DCM and concentrated under reduced pressure to give a residue. The compound 2-[(3S)-3-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]ethyl]pyrrolidin-1-yl]acetic acid (98 mg, 131.0 μmol, 96.8% yield) was obtained as a white solid.

### Step F. Procedure for Preparation of tert-butyl 6-[8-(1,3-benzothiazol-2-ylcarbamoyl)-3,4-dihydro-1H-isoquinolin-2-yl]-3-[3-[2-[(3S)-1-[2-[[3-(2,6-dioxo-3-piperidyl)-1-methyl-indazol-6-yl]amino]-2-oxo-ethyl]pyrrolidin-3-yl]ethoxy]-2-methyl-phenyl]pyridine-2-carboxylate

A mixture of 2-[(3S)-3-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]ethyl]pyrrolidin-1-yl]acetic acid (80 mg, 106.97 μmol, 1 equiv.), 3-(6-amino-1-methyl-indazol-3-yl)piperidine-2,6-dione (27.63 mg, 106.97 μmol, 1 equiv.), EDCI (30.76 mg, 160.45 μmol, 1.5 equiv.) in pyridine (1 mL) was degassed and purged with N₂ three times, and then the mixture was stirred at 25° C. for 2 hours under N₂ atmosphere. The reaction mixture was then diluted with water 5 mL and extracted with ethyl acetate 30 mL (15 mL×2). The combined organic layers were washed with brine 20 mL (10 mL×2), dried over Na₂SO₄, filtered, and concentrated under reduced pressure to give a residue. The compound tert-butyl 6-[8-(1,3-benzothiazol-2-ylcarbamoyl)-3,4-dihydro-1H-isoquinolin-2-yl]-3-[3-[2-[(3S)-1-[2-[[3-(2,6-dioxo-3-piperidyl)-1-methyl-indazol-6-yl]amino]-2-oxo-ethyl]pyrrolidin-3-yl]ethoxy]-2-methyl-phenyl]pyridine-2-carboxylate (150 mg, 74.8 μmol, 69.9% yield, 49.3% purity) was obtained as a red oil.

MS (ESI) m/z: 989.4 [M+H]⁺.

### Step G. Procedure for Preparation of 6-[8-(1,3-benzothiazol-2-ylcarbamoyl)-3,4-dihydro-1H-isoquinolin-2-yl]-3-[3-[2-[(3S)-1-[2-[[3-(2,6-dioxo-3-piperidyl)-1-methyl-indazol-6-yl]amino]-2-oxo-ethyl]pyrrolidin-3-yl]ethoxy]-2-methyl-phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[3-[2-[(3S)-1-[2-[[3-(2,6-dioxo-3-piperidyl)-1-methyl-indazol-6-yl]amino]-2-oxo-ethyl]pyrrolidin-3-yl]ethoxy]-2-methyl-phenyl]pyridine-2-carboxylate (150 mg, 151.80 μmol, 1 equiv.) in TFA (1 mL) and DCM (1 mL) was degassed and purged with N₂ three times, and then the mixture was stirred at 25° C. for 12 hours under N₂ atmosphere. The reaction mixture was diluted with water 5 mL and extracted with ethyl acetate 10 mL (5 mL×2). The combined organic layers were washed with brine 10 mL (5 mL×2), dried over Na₂SO₄, filtered, and concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[2-[(3S)-1-[2-[[3-(2,6-dioxo-3-piperidyl)-1-methyl-indazol-6-yl]amino]-2-oxo-ethyl]pyrrolidin-3-yl]ethoxy]-2-methyl-phenyl]pyridine-2-carboxylic acid (28.7 mg, 29.8 μmol, 19.6% yield, 96.7% purity) as a purple solid.

MS (ESI) m/z: 933.6 [M+H]⁺.

¹H NMR (400 MHz, DMSO-d₆) δ=13.01-12.71 (m, 1H), 10.88 (s, 1H), 10.49-9.89 (m, 1H), 8.14 (s, 1H), 8.06-8.00 (m, 2H), 7.81-7.76 (m, 1H), 7.66-7.61 (m, 2H), 7.48-7.43 (m, 2H), 7.40 (d, J=4.8 Hz, 1H), 7.39-7.32 (m, 2H), 7.16 (d, J=9.2 Hz, 1H), 7.12 (s, 1H), 6.93 (dd, J=8.4, 18.4 Hz, 2H), 6.64 (d, J=7.6 Hz, 1H), 4.98 (s, 2H), 4.32 (dd, J=5.2, 9.6 Hz, 1H), 4.01 (s, 2H), 3.93 (d, J=1.6 Hz, 2H), 3.91 (s, 3H), 3.03 (t, J=6.0 Hz, 4H), 2.71-2.57 (m, 4H), 2.46 (s, 2H), 2.37-2.27 (m, 2H), 2.20-2.11 (m, 2H), 1.91 (s, 5H), 1.68-1.58 (m, 1H)

### Example 55. Preparation of Compound 219b

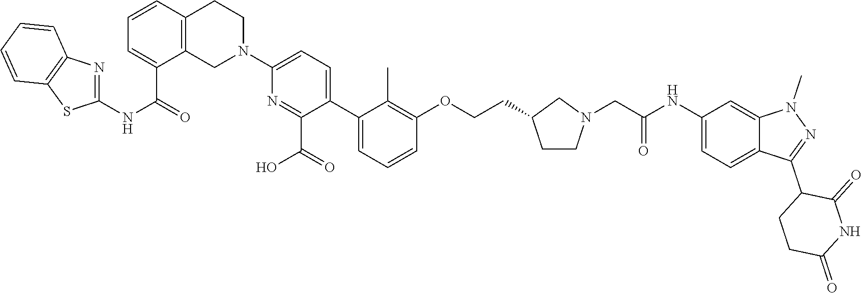 [SMILES: Cc1c(OCC[C@H]2CCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[2-[(3R)-1-[2-[[3-(2,6-dioxo-3-piperidyl)-1-methyl-indazol-6-yl]amino]-2-oxo-ethyl]pyrrolidin-3-yl]ethoxy]-2-methyl-phenyl]pyridine-2-carboxylic acid

### Step A. Procedure for Preparation of tert-butyl (3R)-3-[2-(3-bromo-2-methyl-phenoxy)ethyl]pyrrolidine-1-carboxylate

To a solution of 3-bromo-2-methyl-phenol (764.51 mg, 4.09 mmol, 1.1 equiv.) in THE (32 mL) was added tert-butyl (3R)-3-(2-hydroxyethyl)pyrrolidine-1-carboxylate (800 mg, 3.72 mmol, 1 equiv.), DIAD (1.20 g, 5.95 mmol, 1.16 mL, 1.6 equiv.) and PPh3 (1.56 g, 5.95 mmol, 1.6 equiv.). The mixture was stirred at 25° C. for 8 hours. The reaction mixture was concentrated under reduced pressure to give a residue. The residue was purified by flash silica gel chromatography (Eluent of 0˜37% ethyl acetate/petroleum ether) to give tert-butyl (3R)-3-[2-(3-bromo-2-methyl-phenoxy)ethyl]pyrrolidine-1-carboxylate (400 mg, 822.2 μmol, 22.1% yield, 79% purity) as a white solid.

MS (ESI) m/z: 329.7 [M-56+1]⁺

¹H NMR (400 MHz, CD₃OD) δ=7.14-7.12 (m, 1H), 7.06-7.00 (m, 1H), 6.90-6.88 (m, 1H), 4.08-3.99 (m, 2H), 3.65-3.57 (m, 1H), 3.49-3.44 (m, 1H), 3.38-3.35 (s, 1H), 3.03-2.96 (m, 1H), 2.31 (s, 3H), 2.15-2.06 (m, 1H), 1.94-1.87 (m, 2H), 1.69-1.56 (m, 1H), 1.47 (s, 9H), 1.32-1.24 (m, 1H).

### Step B. Procedure for Preparation of (3R)-3-[2-(3-bromo-2-methyl-phenoxy)ethyl]pyrrolidine

To a solution of tert-butyl (3R)-3-[2-(3-bromo-2-methyl-phenoxy)ethyl]pyrrolidine-1-carboxylate (300 mg, 208.17 μmol, 1 equiv.) in HCl/EtOAc (10 mL). The mixture was stirred at 25° C. for 2 hours. The reaction mixture was concentrated under reduced pressure to give a residue. The residue was used in the next step without purification. The compound (3R)-3-[2-(3-bromo-2-methyl-phenoxy)ethyl] pyrrolidine (180 mg, 561.3 μmol, 71.9% yield) was obtained as a white solid.

MS (ESI) m/z: 285.8 [M+H]⁺

¹H NMR (400 MHz, DMSO-d₆) δ=7.18-7.17 (m, 1H), 7.11 (t, J=8.0 Hz, 1H), 7.02-6.97 (m, 1H), 4.05-4.02 (m, 2H), 3.28-3.18 (m, 1H), 2.83-2.75 (m, 1H), 2.42-2.34 (m, 1H), 2.25 (s, 3H), 2.13-2.09 (m, 1H), 2.00-1.98 (s, 1H), 1.92-1.86 (m, 2H), 1.64-1.54 (m, 1H), 1.25-1.13 (m, 2H).

### Step C. Procedure for Preparation of ethyl 2-[(3R)-3-[2-(3-bromo-2-methyl-phenoxy)ethyl]pyrrolidin-1-yl]acetate

A mixture of (3R)-3-[2-(3-bromo-2-methyl-phenoxy)ethyl]pyrrolidine (160 mg, 479.02 μmol, 96% purity, 1 equiv., HCl), ethyl 2-bromoacetate (80.00 mg, 479.02 μmol, 52.98 μL, 1 equiv.), and K₂CO₃ (198.61 mg, 1.44 mmol, 3 equiv.) in CH₃CN (4 mL) was degassed and purged with N₂ three times, and then the mixture was stirred at 60° C. for 2 hours under N₂ atmosphere. The reaction mixture was concentrated under reduced pressure to give a residue. The residue was purified by flash silica gel chromatography (Eluent of 0˜37% ethyl acetate/petroleum ether) to give ethyl 2-[(3R)-3-[2-(3-bromo-2-methyl-phenoxy)ethyl]pyrrolidin-1-yl]acetate (150 mg, 405.1 μmol, 84.5% yield) as a white solid.

MS (ESI) m/z: 371.9 [M+H]⁺

### Step D. Procedure for Preparation of tert-butyl 6-[8-(1,3-benzothiazol-2-ylcarbamoyl)-3,4-dihydro-1H-isoquinolin-2-yl]-3-[3-[2-[(3R)-1-(2-ethoxy-2-oxo-ethyl)pyrrolidin-3-yl]ethoxy]-2-methyl-phenyl]pyridine-2-carboxylate

A mixture of ethyl 2-[(3R)-3-[2-(3-bromo-2-methyl-phenoxy)ethyl]pyrrolidin-1-yl]acetate (80 mg, 216.05 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (158.81 mg, 259.26 μmol, 1.2 equiv.), Ad₂nBuP Pd G3(cataCXium® A Pd G3) (31.47 mg, 43.21 μmol, 0.2 equiv.), and K₂CO₃ (1.5 M, 216.05 μL, 1.5 equiv.) in 1,4-dioxane (4 mL) was degassed and purged with N₂ three times, and then the mixture was stirred at 100° C. for 1 hour under microwave. The reaction mixture was concentrated under reduced pressure to give a residue. The residue was purified by flash silica gel chromatography (Eluent of 0˜37% ethyl acetate/petroleum ether) to give tert-butyl 6-[8-(1,3-benzothiazol-2-ylcarbamoyl)-3,4-dihydro-1H-isoquinolin-2-yl]-3-[3-[2-[(3R)-1-(2-ethoxy-2-oxo-ethyl)pyrrolidin-3-yl]ethoxy]-2-methyl-phenyl]pyridine-2-carboxylate (80 mg, 103.1 μmol, 47.7% yield) as a white solid.

MS (ESI) m/z: 776.7 [M+H]⁺

### Step E. Procedure for Preparation of 2-[(3R)-3-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]ethyl]pyrrolidin-1-yl]acetic acid

To a solution of tert-butyl 6-[8-(1,3-benzothiazol-2-ylcarbamoyl)-3,4-dihydro-1H-isoquinolin-2-yl]-3-[3-[2-[(3R)-1-(2-ethoxy-2-oxo-ethyl)pyrrolidin-3-yl]ethoxy]-2-methyl-phenyl]pyridine-2-carboxylate (80 mg, 103.10 μmol, 1 equiv.) in THE (2 mL) was added LiOH·H₂O (12.98 mg, 309.30 μmol, 3 equiv.) and H₂O (0.5 mL). The mixture was stirred at 25° C. for 2 hours. The reaction mixture was concentrated under reduced pressure to give a residue. The residue used in the next step without purification. The compound 2-[(3R)-3-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]ethyl]pyrrolidin-1-yl]acetic acid (80 mg, crude) was obtained as a white solid.

MS (ESI) m/z: 748.7 [M+H]⁺

### Step F. Procedure for Preparation of tert-butyl 6-[8-(1,3-benzothiazol-2-ylcarbamoyl)-3,4-dihydro-1H-isoquinolin-2-yl]-3-[3-[2-[(3R)-1-[2-[[3-(2,6-dioxo-3-piperidyl)-1-methyl-indazol-6-yl]amino]-2-oxo-ethyl]pyrrolidin-3-yl]ethoxy]-2-methyl-phenyl]pyridine-2-carboxylate

A mixture of 2-[(3R)-3-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]ethyl]pyrrolidin-1-yl]acetic acid (80 mg, 106.97 μmol, 1 equiv.), 3-(6-amino-1-methyl-indazol-3-yl)piperidine-2,6-dione (33.15 mg, 128.36 μmol, 1.2 equiv.), HATU (40.67 mg, 106.97 μmol, 1 equiv.), DIPEA (41.47 mg, 320.90 μmol, 55.89 μL, 3 equiv.) in DMF (3 mL) was degassed and purged with N₂ three times, and then the mixture was stirred at 25° C. for 2 hours under N₂ atmosphere. The reaction mixture was diluted with water (6 mL) and extracted with ethyl acetate (3 mL×3). The combined organic layers were washed with water (3 mL×3), filtered, and concentrated under reduced pressure to give a residue. The residue was used in the next step without purification. The compound tert-butyl 6-[8-(1,3-benzothiazol-2-ylcarbamoyl)-3,4-dihydro-1H-isoquinolin-2-yl]-3-[3-[2-[(3R)-1-[2-[[3-(2,6-dioxo-3-piperidyl)-1-methyl-indazol-6-yl]amino]-2-oxo-ethyl]pyrrolidin-3-yl]ethoxy]-2-methyl-phenyl]pyridine-2-carboxylate (70 mg, 70.8 μmol, 66.2% yield) was obtained as a white solid.

MS (ESI) m/z: 721.5 [M+H]⁺

### Step G. Procedure for Preparation of 6-[8-(1,3-benzothiazol-2-ylcarbamoyl)-3,4-dihydro-1H-isoquinolin-2-yl]-3-[3-[2-[(3R)-1-[2-[[3-(2,6-dioxo-3-piperidyl)-1-methyl-indazol-6-yl]amino]-2-oxo-ethyl]pyrrolidin-3-yl]ethoxy]-2-methyl-phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[2-[(3R)-1-[2-[[3-(2,6-dioxo-3-piperidyl)-1-methyl-indazol-6-yl]amino]-2-oxo-ethyl]pyrrolidin-3-yl]ethoxy]-2-methyl-phenyl]pyridine-2-carboxylate (70 mg, 70.84 μmol, 1 equiv.) in DCM (1 mL) and TFA (1 mL) was stirred at 40° C. for 1 hour. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[2-[(3R)-1-[2-[[3-(2,6-dioxo-3-piperidyl)-1-methyl-indazol-6-yl]amino]-2-oxo-ethyl]pyrrolidin-3-yl]ethoxy]-2-methyl-phenyl]pyridine-2-carboxylic acid (32.4 mg, 34.5 μmol, 48.8% yield, 99.4% purity) as a white solid.

MS (ESI) m/z: 932.8 [M+H]⁺

¹H NMR (400 MHz, DMSO-d₆) δ=10.87 (s, 1H), 9.89 (s, 1H), 8.14 (s, 1H), 8.04-8.02 (m, 2H), 7.78 (d, J=8.0 Hz, 1H), 7.63-7.59 (m, 2H), 7.48-7.33 (m, 5H), 7.21-7.18 (m, 1H), 7.11-7.06 (m, 1H), 6.97-6.84 (m, 2H), 6.62 (d, J=7.2 Hz, 1H), 5.04-4.95 (m, 2H), 4.33-4.29 (m, 1H), 4.00-3.90 (m, 7H), 3.04-3.02 (m, 2H), 2.98-2.94 (m, 1H), 2.81-2.77 (m, 1H), 2.70-2.60 (m, 4H), 2.40-2.27 (m, 4H), 2.18-2.14 (m, 1H), 2.08-1.98 (m, 1H), 1.93-1.85 (m, 5H), 1.55-1.50 (m, 1H).

### Example 56. Preparation of Compound 226

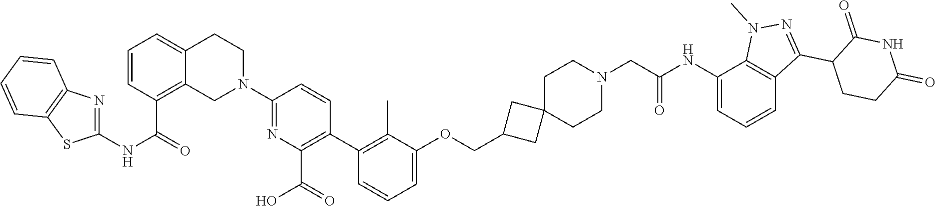 [SMILES: Cc1c(OCC2CC3(CCN(CC(=O)Nc4cccc5c(C6CCC(=O)NC6=O)nn(C)c45)CC3)C2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[[7-[2-[[3-(2,6-dioxo-3-piperidyl)-1-methyl-indazol-7-yl]amino]-2-oxo-ethyl]-7-azaspiro[3.5]nonan-2-yl]methoxy]-2-methyl-phenyl]pyridine-2-carboxylic acid

### Step A. Procedure for Preparation of tert-butyl 2-[(3-bromo-2-methyl-phenoxy)methyl]-7-azaspiro[3.5]nonane-7-carboxylate

A mixture of 3-bromo-2-methyl-phenol (1 g, 5.35 mmol, 1 equiv.), tert-butyl 2-(hydroxymethyl)-7-azaspiro[3.5]nonane-7-carboxylate (1.50 g, 5.88 mmol, 1.1 equiv.), and 2-(tributyl-λ5-phosphanylidene) acetonitrile (1.55 g, 6.42 mmol, 1.2 equiv.) in toluene (10 mL) was degassed and purged with N₂ three times, and then the mixture was stirred at 120° C. for 2 hours under N₂ atmosphere. The reaction mixture was concentrated under reduced pressure to give a residue. The residue was purified by column chromatography (SiO₂, petroleum ether/ethyl acetate=0/1 to 10/1) to give compound tert-butyl 2-[(3-bromo-2-methyl-phenoxy)methyl]-7-azaspiro[3.5]nonane-7-carboxylate (2.35 g, crude) as a green oil.

¹H NMR (400 MHz, CDCl₃) δ=7.15 (d, J=8.0 Hz, 1H), 6.99 (t, J=8.0 Hz, 1H), 6.76 (d, J=8.4 Hz, 1H), 3.91 (d, J=6.0 Hz, 2H), 3.42-3.35 (m, 2H), 3.34-3.26 (m, 2H), 2.83-2.67 (m, 1H), 2.33 (s, 3H), 2.03-1.94 (m, 2H), 1.78-1.69 (m, 2H), 1.66-1.60 (m, 2H), 1.52-1.49 (m, 2H), 1.46 (s, 9H).

### Step B. Procedure for Preparation of 2-[(3-bromo-2-methyl-phenoxy)methyl]-7-azaspiro[3.5]nonane

To a solution of tert-butyl 2-[(3-bromo-2-methyl-phenoxy)methyl]-7-azaspiro[3.5]nonane-7-carboxylate (1.85, 4.36 mmol, 1 equiv.) in DCM (10 mL) was added HCl/1,4-dioxane (4 M, 10 mL, 9.18 equiv.). The mixture was stirred at 25° C. for 1 hour. The reaction mixture was concentrated under reduced pressure to give a residue. The residue was purified by column chromatography (SiO₂, DCM:MeOH=0/1 to 10/1) to give compound 2-[(3-bromo-2-methyl-phenoxy)methyl]-7-azaspiro[3.5]nonane (1.4 g, 4.32 mmol, 99.04% yield) as a white solid.

¹H NMR (400 MHz, DMSO-d₆) δ=7.18-7.14 (m, 1H), 7.09 (t, J=8.0 Hz, 1H), 6.96 (d, J=8.0 Hz, 1H), 3.96 (d, J=6.0 Hz, 2H), 3.02-2.97 (m, 2H), 2.93-2.88 (m, 2H), 2.74-2.66 (m, 1H), 2.24 (s, 3H), 2.01-1.92 (m, 2H), 1.81-1.76 (m, 2H), 1.75-1.70 (m, 2H), 1.70-1.67 (m, 2H).

### Step C. Procedure for Preparation of ethyl 2-[2-[(3-bromo-2-methyl-phenoxy)methyl]-7-azaspiro[3.5]nonan-7-yl]acetate

To a solution of 2-[(3-bromo-2-methyl-phenoxy)methyl]-7-azaspiro[3.5]nonane (500 mg, 1.54 mmol, 1 equiv.) and ethyl 2-bromoacetate (257.51 mg, 1.54 mmol, 170.54 μL, 1 equiv.) in CH₃CN (5 mL) was added K₂CO₃ (1.07 g, 7.71 mmol, 5 equiv.). The mixture was stirred at 60° C. for 12 hours. The reaction mixture was filtered and concentrated under reduced pressure to give a residue. The residue was purified by prep-TLC (SiO₂, DCM:MeOH=10:1) to give compound ethyl 2-[2-[(3-bromo-2-methyl-phenoxy)methyl]-7-azaspiro[3.5]nonan-7-yl]acetate (240 mg, 584.8 μmol, 37.9% yield) as a colorless oil.

¹H NMR (400 MHz, CDCl₃) δ=7.15 (d, J=8.0 Hz, 1H), 6.99 (t, J=8.0 Hz, 1H), 6.76 (d, J=8.4 Hz, 1H), 4.25-4.15 (m, 2H), 3.90 (d, J=6.4 Hz, 2H), 3.20 (s, 2H), 2.80-2.66 (m, 1H), 2.62-2.39 (m, 4H), 2.32 (s, 3H), 2.04-1.90 (m, 2H), 1.76 (t, J=4.4 Hz, 2H), 1.58 (s, 4H), 1.28 (t, J=7.2 Hz, 3H).

### Step D. Procedure for Preparation of tert-butyl 6-[8-(1,3-benzothiazol-2-ylcarbamoyl)-3,4-dihydro-1H-isoquinolin-2-yl]-3-[3-[[7-(2-ethoxy-2-oxo-ethyl)-7-azaspiro[3.5]nonan-2-yl]methoxy]-2-methyl-phenyl]pyridine-2-carboxylate

Ethyl 2-[2-[(3-bromo-2-methyl-phenoxy)methyl]-7-azaspiro[3.5]nonan-7-yl]acetate (240 mg, 584.87 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (358.26 mg, 584.87 μmol, 1 equiv.) K₂CO₃ (1.5 M, 1.17 mL, 3 equiv.) and Ad₂nBuP Pd G₃(cataCXium® A Pd G₃) (85.19 mg, 116.97 μmol, 0.2 equiv.) were taken up into a microwave tube in 1,4-dioxane (3 mL). The sealed tube was heated at 100° C. for 1 hour under microwave. The reaction mixture was filtered and concentrated under reduced pressure to give a residue. The residue was purified by column chromatography (SiO₂, petroleum ether/ethyl acetate=0/1 to 1/1) to give compound tert-butyl 6-[8-(1,3-benzothiazol-2-ylcarbamoyl)-3,4-dihydro-1H-isoquinolin-2-yl]-3-[3-[[7-(2-ethoxy-2-oxo-ethyl)-7-azaspiro[3.5]nonan-2-yl]methoxy]-2-methyl-phenyl]pyridine-2-carboxylate (240 mg, 294.1 μmol, 50.2% yield) as a yellow solid.

¹H NMR (400 MHz, CDCl₃) δ=7.86 (d, J=7.6 Hz, 1H), 7.72 (d, J=8.0 Hz, 1H), 7.57 (d, J=7.2 Hz, 1H), 7.47-7.41 (m, 1H), 7.38 (d, J=8.4 Hz, 2H), 7.35-7.30 (m, 2H), 7.14-7.06 (m, 1H), 6.90 (d, J=8.8 Hz, 1H), 6.80 (d, J=8.0 Hz, 1H), 6.70 (d, J=7.6 Hz, 1H), 5.16-4.90 (m, 2H), 4.23-4.16 (m, 2H), 4.11 (t, J=6.0 Hz, 2H), 3.94 (d, J=6.0 Hz, 2H), 3.32-3.14 (m, 2H), 3.07 (t, J=5.6 Hz, 2H), 2.77-2.68 (m, 1H), 2.64-2.36 (m, 4H), 2.00 (s, 3H), 1.95 (d, J=9.6 Hz, 2H), 1.82-1.66 (m, 6H), 1.28 (s, 3H), 1.15 (s, 9H).

### Step E. Procedure for Preparation of 2-[2-[[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]methyl]-7-azaspiro[3.5]nonan-7-yl]acetic acid

To a solution of tert-butyl 6-[8-(1,3-benzothiazol-2-ylcarbamoyl)-3,4-dihydro-1H-isoquinolin-2-yl]-3-[3-[[7-(2-ethoxy-2-oxo-ethyl)-7-azaspiro[3.5]nonan-2-yl]methoxy]-2-methyl-phenyl]pyridine-2-carboxylate (240 mg, 294.11 μmol, 1 equiv.) in THE (0.5 mL) and H₂O (0.5 mL) was added LiOH·H₂O (61.71 mg, 1.47 mmol, 5 equiv.). The mixture was stirred at 25° C. for 12 hours. The reaction mixture was concentrated under reduced pressure to remove THE and adjusted pH to 5 with HCl (1M). The mixture was then filtered concentrated under reduced pressure to give compound 2-[2-[[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]methyl]-7-azaspiro[3.5]nonan-7-yl]acetic acid (270 mg, crude) as a yellow solid.

MS (ESI) m/z: 788.9 [M+H]⁺

### Step F. Procedure for Preparation of tert-butyl 6-[8-(1,3-benzothiazol-2-ylcarbamoyl)-3,4-dihydro-1H-isoquinolin-2-yl]-3-[3-[[7-[2-[[3-(2,6-dioxo-3-piperidyl)-1-methyl-indazol-7-yl]amino]-2-oxo-ethyl]-7-azaspiro[3.5]nonan-2-yl]methoxy]-2-methyl-phenyl]pyridine-2-carboxylate

To a solution of 2-[2-[[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]methyl]-7-azaspiro[3.5]nonan-7-yl]acetic acid (80 mg, 101.53 μmol, 1 equiv.) and 3-(7-amino-1-methyl-indazol-3-yl)piperidine-2,6-dione (31.47 mg, 121.83 μmol, 1.2 equiv.) in pyridine (1 mL) was added EDCI (29.19 mg, 152.29 μmol, 1.5 equiv.). The mixture was stirred at 25° C. for 2 hours. The reaction mixture was quenched by addition water 2 mL, and then diluted with EtOAc 2 mL and extracted with EtOAc (2 mL×3). The combined organic layers were washed with brine (2 mL×3), dried over anhydrous sodium sulfate, filtered and concentrated under reduced pressure to give compound tert-butyl 6-[8-(1,3-benzothiazol-2-ylcarbamoyl)-3,4-dihydro-1H-isoquinolin-2-yl]-3-[3-[[7-[2-[[3-(2,6-dioxo-3-piperidyl)-1-methyl-indazol-7-yl]amino]-2-oxo-ethyl]-7-azaspiro[3.5]nonan-2-yl]methoxy]-2-methyl-phenyl]pyridine-2-carboxylate (100 mg, crude) as a brown oil.

MS (ESI) m/z: 1028.8 [M+H]⁺

### Step G. Procedure for Preparation of 6-[8-(1,3-benzothiazol-2-ylcarbamoyl)-3,4-dihydro-1H-isoquinolin-2-yl]-3-[3-[[7-[2-[[3-(2,6-dioxo-3-piperidyl)-1-methyl-indazol-7-yl]amino]-2-oxo-ethyl]-7-azaspiro[3.5]nonan-2-yl]methoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[[7-[2-[[3-(2,6-dioxo-3-piperidyl)-1-methyl-indazol-7-yl]amino]-2-oxo-ethyl]-7-azaspiro[3.5]nonan-2-yl]methoxy]-2-methyl-phenyl]pyridine-2-carboxylate (100 mg, 97.26 μmol, 1 equiv.) in DCM (0.5 mL) was added TFA (1.54 g, 13.51 mmol, 1.0 mL, 138.87 equiv.). The mixture was stirred at 25° C. for 12 hours. The reaction mixture was filtered and concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[[7-[2-[[3-(2,6-dioxo-3-piperidyl)-1-methyl-indazol-7-yl]amino]-2-oxo-ethyl]-7-azaspiro[3.5]nonan-2-yl]methoxy]-2-methyl-phenyl]pyridine-2-carboxylic acid (26.7 mg, 27.3 μmol, 28.1% yield, 99.3% purity) as a yellow solid.

MS (ESI) m/z: 972.7 [M+H]⁺

¹H NMR (400 MHz, DMSO-d₆) δ=10.89 (s, 1H), 9.84 (s, 1H), 8.06-7.98 (m, 1H), 7.82-7.74 (m, 1H), 7.62 (d, J=6.4 Hz, 1H), 7.56 (d, J=8.0 Hz, 1H), 7.50-7.40 (m, 3H), 7.39-7.29 (m, 3H), 7.07 (t, J=8.0 Hz, 2H), 6.98-6.82 (m, 2H), 6.64 (d, J=8.0 Hz, 1H), 4.98 (s, 2H), 4.43-4.31 (m, 1H), 4.11 (s, 3H), 3.93 (d, J=5.6 Hz, 4H), 3.13 (s, 2H), 3.02 (s, 2H), 2.82-2.67 (m, 2H), 2.65-2.60 (m, 1H), 2.46-2.34 (m, 4H), 2.23-2.11 (m, 2H), 1.96-1.87 (m, 5H), 1.75-1.65 (m, 4H), 1.61 (d, J=4.8 Hz, 2H).

### Example 57. Preparation of Compound 229

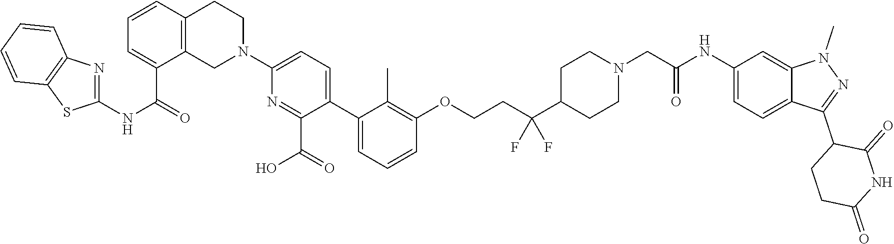 [SMILES: Cc1c(OCCC(F)(F)C2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-6-yl)amino)-2-oxoethyl)piperidin-4-yl)-3,3-difluoropropoxy)-2-methylphenyl)picolinic acid

### Step A. Procedure for Preparation of tert-butyl 4-(methoxy(methyl)carbamoyl)piperidine-1-carboxylate

A mixture of 1-(tert-butoxycarbonyl)piperidine-4-carboxylic acid (50.0 g, 218 mmol, 1.0 equiv.), N,O-dimethylhydroxylamine (27.7 g, 283 mmol, 1.3 equiv.), EDCI (100 g, 523 mmol, 2.4 equiv.), NMM (33.1 g, 327 mmol, 36.0 mL, 1.5 equiv.), and HOBt (14.7 g, 109 mmol, 0.50 equiv.) in CH₂Cl₂ (500 mL) was stirred at 20° C. for 5 hours. After completion, the reaction mixture was diluted with H₂O (500 mL) and extracted with CH₂Cl₂ (400 mL×2). The combined organic layers were dried over anhydrous sodium sulfate, filtered, and concentrated under reduced pressure. The residue was purified by column chromatography (SiO₂, dichloromethane/methanol=100/1 to 20/1) to give compound tert-butyl 4-(methoxy(methyl)carbamoyl)piperidine-1-carboxylate (52.3 g, 192 mmol, 88.1% yield) as a yellow oil.

¹H NMR (400 MHz) δ=4.30-4.00 (m, 2H), 3.71 (s, 3H), 3.18 (s, 3H), 2.90-2.67 (m, 3H), 1.75-1.62 (m, 4H), 1.45 (s, 9H).

### Step B. Procedure for Preparation of tert-butyl 4-(but-3-enoyl)piperidine-1-carboxylate

To a solution of tert-butyl 4-(methoxy(methyl)carbamoyl)piperidine-1-carboxylate (15.0 g, 55.1 mmol, 1.0 equiv.) in THE (200 mL) was added dropwise allyl magnesium bromide (1 M, 66.1 mL, 1.2 equiv.) at 0° C. After addition, the mixture was stirred 20° C. for 3 hours. The reaction mixture was quenched by addition saturated NH₄Cl aqueous solution (200 mL) at 0° C. and then extracted with CH₂Cl₂ (200 mL×2). The combined organic layers were dried over anhydrous sodium sulfate, filtered, and concentrated under reduced pressure. The residue was purified by column chromatography (SiO₂, dichloromethane/methanol=100/1 to 10/1) to give tert-butyl 4-(but-3-enoyl)piperidine-1-carboxylate (14.0 g, 49.7 mmol, 90.3% yield, 90% purity) as a brown oil.

¹H NMR (400 MHz, CDCl₃) δ=5.99-5.81 (m, 1H), 5.22-5.06 (m, 2H), 4.11 (d, J=7.2 Hz, 2H), 3.22 (d, J=6.8 Hz, 2H), 2.77 (t, J=12.0 Hz, 2H), 2.58-2.47 (m, 1H), 1.84-1.75 (m, 2H), 1.58-1.48 (m, 2H), 1.44 (s, 9H).

### Step C. Procedure for Preparation of tert-butyl 4-(1,1-difluorobut-3-en-1-yl)piperidine-1-carboxylate

To a solution of tert-butyl 4-(but-3-enoyl)piperidine-1-carboxylate (14.0 g, 55.3 mmol, 1.0 equiv.) in CH₂Cl₂ (200 mL) was added DAST (44.5 g, 276 mmol, 36.5 mL, 5.0 equiv.) dropwise at 0° C. After that, the mixture was stirred at 20° C. for 5 hours. The reaction mixture was quenched by addition saturated NaHCO₃ aqueous solution (200 mL) at 0° C. and then extracted with CH₂Cl₂ (200 mL×3). The combined organic layers were dried over anhydrous sodium sulfate, filtered, and concentrated under reduced pressure. The residue was purified by column chromatography (SiO₂, petroleum ether/ethyl acetate=50/1 to 5/1) to give tert-butyl 4-(1,1-difluorobut-3-en-1-yl)piperidine-1-carboxylate (4.40 g, 16.0 mmol, 28.9% yield) as a colorless oil.

¹H NMR (400 MHz, CDCl₃) δ=5.90-5.70 (m, 1H), 5.35-5.01 (m, 2H), 4.32-4.14 (m, 2H), 2.80-2.49 (m, 4H), 1.96-1.85 (m, 1H), 1.76 (d, J=12.8 Hz, 2H), 1.46 (s, 9H), 1.44-1.37 (m, 2H).

### Step D. Procedure for Preparation of tert-butyl 4-(1,1-difluoro-3-hydroxypropyl)piperidine-1-carboxylate

Ozone was bubbled into a solution of tert-butyl 4-(1,1-difluorobut-3-en-1-yl)piperidine-1-carboxylate (4.40 g, 16.0 mmol, 1.0 equiv.) in CH₂Cl₂ (30.0 mL) and ethanol (10.0 mL) at −78° C. for 1 hour until the solution became blue. After excess ozone was degassed and purged by nitrogen, NaBH₄ (1.81 g, 47.9 mmol, 3.0 equiv.) was added in portions at 0° C., and the reaction was stirred at 0° C. for 1 hour. The reaction mixture was quenched by addition saturated ammonium chloride aqueous solution (80 mL) at 0° C., and then extracted with CH₂Cl₂ (40 mL×2). The combined organic layers were dried over anhydrous sodium sulfate, filtered, and concentrated under reduced pressure to give a residue. The residue was purified by column chromatography (SiO₂, petroleum ether/ethyl acetate=40/1 to 1/1) to give compound tert-butyl 4-(1,1-difluoro-3-hydroxypropyl)piperidine-1-carboxylate (3.50 g, 11.3 mmol, 70.6% yield, 90.0% purity) as a yellow oil.

¹H NMR (400 MHz, CDCl₃) δ=4.25-4.04 (m, 2H), 3.90-3.77 (m, 2H), 2.71-2.48 (m, 2H), 2.10-1.96 (m, 2H), 1.92-1.82 (m, 1H), 1.71 (d, J=13.2 Hz, 2H), 1.39 (s, 9H), 1.37-1.27 (m, 2H).

### Step E. Procedure for Preparation of tert-butyl 4-(3-(3-bromo-2-methylphenoxy)-1,1-difluoropropyl)piperidine-1-carboxylate

To a solution of tert-butyl 4-(1,1-difluoro-3-hydroxypropyl)piperidine-1-carboxylate (3.50 g, 12.5 mmol, 1.0 equiv.) in toluene (40.0 mL) was added 3-bromo-2-methylphenol (2.58 g, 13.8 mmol, 1.1 equiv.) and CMPB (6.05 g, 25.1 mmol, 2.0 equiv.). The reaction was stirred at 120° C. for 2 hours. After completion, the mixture was concentrated under reduced pressure and the residue was purified by column chromatography (SiO₂, petroleum ether/ethyl acetate=30/1 to 3/1) to give tert-butyl 4-(3-(3-bromo-2-methylphenoxy)-1,1-difluoropropyl)piperidine-1-carboxylate (2.4 g, 3.5 mmol, 28.3% yield, 63.6% purity) as a yellow oil.

MS (ESI) m/z: 448.3 [M+H]⁺

¹H NMR (400 MHz, CDCl₃) δ=7.10 (d, J=8.0 Hz, 1H), 6.94 (t, J=8.4 Hz, 1H), 6.71 (d, J=8.4 Hz, 1H), 4.19-4.06 (m, 4H), 2.64-2.54 (m, 2H), 2.38-2.27 (m, 2H), 2.23 (s, 3H), 1.97-1.89 (m, 1H), 1.76 (d, J=13.2 Hz, 2H), 1.40-1.39 (m, 11H).

### Step F. Procedure for Preparation of 4-(3-(3-bromo-2-methylphenoxy)-1,1-difluoropropyl)piperidine

A mixture of tert-butyl 4-(3-(3-bromo-2-methylphenoxy)-1,1-difluoropropyl)piperidine-1-carboxylate (2.26 g, 5.04 mmol, 1.0 equiv.) in HCl/1,4-dioxane (20.0 mL) was stirred at 25° C. for 1 hour. After completion, the reaction mixture was concentrated under reduced pressure to give 4-(3-(3-bromo-2-methylphenoxy)-1,1-difluoropropyl)piperidine (1.76 g, crude) as a yellow oil.

MS (ESI) m/z: 347.8 [M+H]⁺

### Step G. Procedure for Preparation of ethyl 2-(4-(3-(3-bromo-2-methylphenoxy)-1,1-difluoropropyl)piperidin-1-yl)acetate

To a solution of 4-(3-(3-bromo-2-methylphenoxy)-1,1-difluoropropyl)piperidine (1.76 g, 5.05 mmol, 1.0 equiv.) and ethyl 2-bromoacetate (802 mg, 4.80 mmol, 531 μL, 0.95 equiv.) in CH₃CN (20 mL) was added K₂CO₃ (1.40 g, 10.1 mmol, 2.0 equiv.). The mixture was stirred at 60° C. for 2 hours. After completion, the reaction mixture was diluted with H₂O (30 mL) and extracted with CH₂Cl₂ (30 mL×2). The combined organic layers were dried over anhydrous sodium sulfate, filtered, and concentrated under reduced pressure to give a residue. The residue was purified by column chromatography (SiO₂, petroleum ether/ethyl acetate=30/1 to 3/1) to give ethyl 2-(4-(3-(3-bromo-2-methylphenoxy)-1,1-difluoropropyl)piperidin-1-yl)acetate (1.10 g, 2.45 mmol, 48.5% yield, 96.8% purity) as a yellow oil.

MS (ESI) m/z: 435.9 [M+H]⁺

¹H NMR (400 MHz, CDCl₃) δ=7.10 (d, J=8.0 Hz, 1H), 6.94 (t, J=8.0 Hz, 1H), 6.71 (d, J=8.0 Hz, 1H), 4.18-4.05 (m, 4H), 3.15 (s, 2H), 2.97 (d, J=11.2 Hz, 2H), 2.38-2.25 (m, 2H), 2.23 (s, 3H), 2.11 (t, J=11.2 Hz, 2H), 1.85-1.72 (m, 3H), 1.69-1.58 (m, 2H), 1.21 (t, J=7.2 Hz, 3H).

### Step H. Procedure for Preparation of tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)piperidin-4-yl)-3,3-difluoropropoxy)-2-methylphenyl)picolinate

A mixture of ethyl 2-(4-(3-(3-bromo-2-methylphenoxy)-1,1-difluoropropyl)piperidin-1-yl)acetate (313 mg, 684 μmol, 95% purity, 1.0 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (440 mg, 718 μmol, 1.05 equiv.), Ad₂nBuP Pd G₃ (cataCXium® A Pd G₃) (99.6 mg, 137 μmol, 0.20 equiv.), K₃PO₄ (436 mg, 2.05 mmol, 3.0 equiv.) and H₂O (0.30 mL) in 1,4-dioxane (3.00 mL) was degassed and purged with nitrogen three times, and then the mixture was stirred at 90° C. for 2 hours under nitrogen atmosphere. The reaction mixture was diluted with H₂O (10 mL) and extracted with CH₂Cl₂ (10 mL×3). The combined organic layers were dried over anhydrous sodium sulfate, filtered, and concentrated under reduced pressure to give a residue. The residue was purified by column chromatography (SiO₂, petroleum ether/ethyl acetate=30/1 to 1/1) to give compound tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)piperidin-4-yl)-3,3-difluoropropoxy)-2-methylphenyl)picolinate (357 mg, 372 μmol, 54.4% yield, 87.6% purity) as a yellow solid.

MS (ESI) m/z: 840.7 [M+H]⁺

¹H NMR (400 MHz, CDCl₃) δ=7.86 (d, J=7.6 Hz, 1H), 7.66 (d, J=8.0 Hz, 1H), 7.57 (d, J=8.0 Hz, 1H), 7.44-7.28 (m, 5H), 7.12 (t, J=8.0 Hz, 1H), 6.90 (d, J=8.8 Hz, 1H), 6.83 (d, J=8.0 Hz, 1H), 6.73 (d, J=8.0 Hz, 1H), 5.16-4.93 (m, 2H), 4.26-4.14 (m, 4H), 4.13-4.08 (m, 2H), 3.24 (s, 2H), 3.11-3.01 (m, 4H), 2.45-2.33 (m, 2H), 2.29-2.12 (m, 2H), 1.98 (s, 3H), 1.89-1.83 (m, 2H), 1.79-1.70 (m, 2H), 1.28-1.25 (m, 4H), 1.15 (s, 9H).

### Step I. Procedure for preparation of 2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)-1,1-difluoropropyl)piperidin-1-yl)acetic acid

To a solution of tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)piperidin-4-yl)-3,3-difluoropropoxy)-2-methylphenyl)picolinate (307 mg, 365 μmol, 1.0 equiv.) in THE (2.00 mL) and H₂O (2.00 mL) was added LiOH·H₂O (43.8 mg, 1.83 mmol, 5.0 equiv.). The mixture was stirred at 20° C. for 2 hours. The reaction mixture was concentrated under reduced pressure to give compound 2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)-1,1-difluoropropyl)piperidin-1-yl)acetic acid (190 mg, crude) as a yellow solid.

MS (ESI) m/z: 812.4 [M+H]⁺

### Step J Procedure for preparation of tert-butyl 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-6-yl)amino)-2-oxoethyl)piperidin-4-yl)-3,3-difluoropropoxy)-2-methylphenyl)picolinate

To a solution of 2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)-1,1-difluoropropyl)piperidin-1-yl)acetic acid (100 mg, 123 μmol, 1.0 equiv.) in DMF (1.00 mL) were added 3-(6-amino-1-methyl-1H-indazol-3-yl)piperidine-2,6-dione (35.0 mg, 135 μmol, 1.1 equiv.), HATU (56.2 mg, 148 μmol, 1.2 equiv.), and DIEA (47.8 mg, 369 μmol, 64.4 μL, 3.0 equiv.). The mixture was stirred at 20° C. for 1 hour. After completion, the reaction mixture was diluted with water (3 mL) and then extracted with CH₂Cl₂ (3 mL×2). The combined organic layers were washed with brine (3 mL×3), dried over anhydrous sodium sulfate, filtered, and concentrated under reduced pressure to give tert-butyl 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-6-yl)amino)-2-oxoethyl)piperidin-4-yl)-3,3-difluoropropoxy)-2-methylphenyl)picolinate (100 mg, crude) as a brown solid.

MS (ESI) m/z: 1052.8 [M+H]⁺

### Step K. Procedure for Preparation of 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-6-yl)amino)-2-oxoethyl)piperidin-4-yl)-3,3-difluoropropoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-6-yl)amino)-2-oxoethyl)piperidin-4-yl)-3,3-difluoropropoxy)-2-methylphenyl)picolinate (100 mg, 95.0 μmol, 1.0 equiv.) in CH₂Cl₂ (1.00 mL) was added TFA (3.08 g, 27.0 mmol, 2 mL, 284 equiv.). The mixture was stirred at 20° C. for 6 hours. After completion, the reaction mixture was concentrated under reduced pressure and the residue was purified by prep-HPLC to give 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-6-yl)amino)-2-oxoethyl)piperidin-4-yl)-3,3-difluoropropoxy)-2-methylphenyl)picolinic acid (23.9 mg, 23.8 μmol, 25.0% yield, 98.7% purity) as a white solid.

MS (ESI) m/z: 996.3 [M+H]⁺

¹H NMR (400 MHz, DMSO-d₆) δ=13.11-12.55 (m, 1H), 10.88 (s, 1H), 10.08-9.67 (m, 1H), 8.03 (d, J=6.4 Hz, 2H), 7.79 (d, J=8.0 Hz, 1H), 7.67-7.57 (m, 2H), 7.52-7.41 (m, 3H), 7.41-7.31 (m, 2H), 7.20 (d, J=8.4 Hz, 1H), 7.16-7.07 (m, 1H), 7.01-6.89 (m, 2H), 6.66 (d, J=7.6 Hz, 1H), 4.98 (s, 2H), 4.38-4.27 (m, 1H), 4.16 (t, J=6.0 Hz, 2H), 3.92 (s, 5H), 3.21-3.10 (m, 2H), 3.07-2.90 (m, 4H), 2.66-2.56 (m, 2H), 2.48-2.33 (m, 3H), 2.27-2.06 (m, 3H), 2.04-1.94 (m, 1H), 1.90 (s, 3H), 1.88-1.73 (m, 2H), 1.70-1.50 (m, 2H).

### Example 58. Preparation of Compound 235

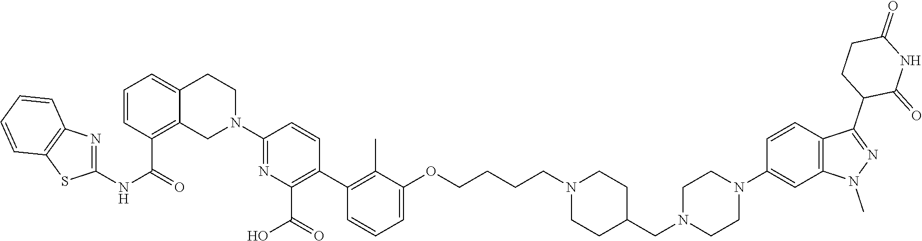 [SMILES: Cc1c(OCCCCN2CCC(CN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[4-[4-[[4-[3-(2,6-dioxo-3-piperidyl)-1-methyl-indazol-6-yl]piperazin-1-yl]methyl]-1-piperidyl]butoxy]-2-methyl-phenyl]pyridine-2-carboxylic acid

### Step A. Procedure for Preparation of 1-bromo-3-(4-bromobutoxy)-2-methyl-benzene

A mixture of 3-bromo-2-methyl-phenol (3.0 g, 16.04 mmol, 1.0 equiv.), 1,4-dibromobutane (10.39 g, 48.12 mmol, 5.80 mL, 3.0 equiv.), Cs₂CO₃ (15.68 g, 48.12 mmol, 3.0 equiv.), and KI (7.99 g, 48.12 mmol, 3.0 equiv.) in CH₃CN (30 mL) was stirred at 25° C. for 5 hours. The reaction mixture was concentrated under reduced pressure to give 1-bromo-3-(4-bromobutoxy)-2-methyl-benzene (3.3 g, 10.2 mmol, 63.9% yield) colorless oil.

### Step B. Procedure for Preparation of [1-[4-(3-bromo-2-methyl-phenoxy) butyl]-4-piperidyl]methanol

A mixture of 1-bromo-3-(4-bromobutoxy)-2-methyl-benzene (1.0 g, 3.11 mmol, 1.0 equiv.), 4-piperidylmethanol (565.03 mg, 3.73 mmol, 1.2 equiv., HCl), K₂CO₃ (1.29 g, 9.32 mmol, 3.0 equiv.), and KI (257.74 mg, 1.55 mmol, 0.5 equiv.) in CH₃CN (30 mL) was stirred at 60° C. for 12 hours. The reaction mixture was concentrated under reduced pressure to give a residue. The residue was purified by reverse-phase HPLC (0.1% NH₃H₂O) to give [1-[4-(3-bromo-2-methyl-phenoxy) butyl]-4-piperidyl] methanol (666 mg, 1.8 mmol, 58.3% yield, 97% purity) as a primrose yellow solid.

MS (ESI) m/z: 357.9 [M+H]⁺.

### Step C. Procedure for Preparation of 1-[4-(3-bromo-2-methyl-phenoxy)butyl]piperidine-4-carbaldehyde

A solvent of DMSO (350.87 mg, 4.49 mmol, 350.87 μL, 4.0 equiv.) in DCM (4 mL) was added dropwise to a solution of oxalyl dichloride (284.99 mg, 2.25 mmol, 196.54 μL, 2.0 equiv.) in DCM (0.5 mL) at −70° C. under N₂ atmosphere. The mixture was stirred at −70° C. for 30 minutes. After which time [1-[4-(3-bromo-2-methyl-phenoxy) butyl]-4-piperidyl] methanol (400 mg, 1.12 mmol, 1.0 equiv.) in DCM (0.5 mL) was added dropwise at −70° C. The solution was stirred at −70° C. for 30 minutes. Then TEA (681.61 mg, 6.74 mmol, 937.56 μL, 6.0 equiv.) was added into the solution. The solution was stirred at 25° C. for 1 hour under N₂ atmosphere. The reaction mixture was diluted with water (20 mL) and extracted with DCM 60 (20 mL×3). The combined organic layers were washed with brine (10 mL×2), dried over Na₂SO₄, filtered, and concentrated under reduced pressure to give 1-[4-(3-bromo-2-methyl-phenoxy)butyl]piperidine-4-carbaldehyde (300 mg, 846.7 μmol, 75.4% yield) as brown oil.

MS (ESI) m/z: 355.1 [M+H]⁺.

### Step D. Procedure for Preparation of 3-[6-[4-[[1-[4-(3-bromo-2-methyl-phenoxy)butyl]-4-piperidyl]methyl]piperazin-1-yl]-1-methyl-indazol-3-yl]piperidine-2,6-dione

To a mixture of 1-[4-(3-bromo-2-methyl-phenoxy)butyl]piperidine-4-carbaldehyde (100 mg, 282.26 μmol, 1 equiv.), 3-(1-methyl-6-piperazin-1-yl-indazol-3-yl)piperidine-2,6-dione (110.89 mg, 338.71 μmol, 1.2 equiv.) and CH₃COOH (169.50 ug, 2.82 μmol, 0.161 μL, 0.01 equiv.) in a solvent of DCM (1 mL) and t-BuOH (1.0 mL) was added NaBH(OAc)₃ (179.47 mg, 846.79 μmol, 3.0 equiv.). Then the mixture was stirred at 25° C. for 2.5 hours. The reaction mixture was concentrated under reduced pressure to give a residue. The residue was purified by prep-TLC (SiO₂, DCM:MeOH=10:1) to give 3-[6-[4-[[1-[4-(3-bromo-2-methyl-phenoxy)butyl]-4-piperidyl]methyl]piperazin-1-yl]-1-methyl-indazol-3-yl]piperidine-2,6-dione (80 mg, 81.5 μmol, 28.8% yield, 67.8% purity) as a white solid.

MS (ESI) m/z: 667.2 [M+H]⁺.

### Step E. Procedure for Preparation of tert-butyl 6-[8-(1,3-benzothiazol-2-ylcarbamoyl)-3,4-dihydro-1H-isoquinolin-2-yl]-3-[3-[4-[4-[[4-[3-(2,6-dioxo-3-piperidyl)-1-methyl-indazol-6-yl]piperazin-1-yl]methyl]-1-piperidyl]butoxy]-2-methyl-phenyl]pyridine-2-carboxylate

A mixture of 3-[6-[4-[[1-[4-(3-bromo-2-methyl-phenoxy)butyl]-4-piperidyl]methyl]piperazin-1-yl]-1-methyl-indazol-3-yl]piperidine-2,6-dione (80 mg, 120.18 μmol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (95.70 mg, 156.24 μmol, 1.3 equiv.), [2-(2-aminophenyl)phenyl]palladium(1+); bis(1-adamantyl)-butyl-phosphane; methanesulfonate (8.75 mg, 12.02 μmol, 0.1 equiv.), and KF (34.91 mg, 600.91 μmol, 14.08 μL, 5.0 equiv.) in a solvent of dioxane (2.0 mL) was stirred at 100° C. for 1 hour under microwave. The reaction mixture was concentrated under reduced pressure to give a residue, which was purified by prep-TLC (SiO₂, DCM:MeOH=10:1) to give tert-butyl 6-[8-(1,3-benzothiazol-2-ylcarbamoyl)-3,4-dihydro-1H-isoquinolin-2-yl]-3-[3-[4-[4-[[4-[3-(2,6-dioxo-3-piperidyl)-1-methyl-indazol-6-yl]piperazin-1-yl]methyl]-1-piperidyl]butoxy]-2-methyl-phenyl]pyridine-2-carboxylate (40 mg, 27.3 μmol, 22.7% yield, 73.3% purity) as brown oil.

MS (ESI) m/z: 1071.6 [M+H]⁺.

### Step F. Procedure for Preparation of 6-[8-(1,3-benzothiazol-2-ylcarbamoyl)-3,4-dihydro-1H-isoquinolin-2-yl]-3-[3-[4-[4-[[4-[3-(2,6-dioxo-3-piperidyl)-1-methyl-indazol-6-yl]piperazin-1-yl]methyl]-1-piperidyl]butoxy]-2-methyl-phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[3-[4-[4-[[4-[3-(2,6-dioxo-3-piperidyl)-1-methyl-indazol-6-yl]piperazin-1-yl]methyl]-1-piperidyl]butoxy]-2-methyl-phenyl]pyridine-2-carboxylate (30 mg, 28.00 μmol, 1.0 equiv.) in TFA (0.8 mL) and DCM (0.8 mL) was stirred at 25° C. for 12 hours. The reaction mixture was concentrated under reduced pressure to give a residue, which was purified by reverse-phase HPLC to give the compound 6-[8-(1,3-benzothiazol-2-ylcarbamoyl)-3,4-dihydro-1H-isoquinolin-2-yl]-3-[3-[4-[4-[[4-[3-(2,6-dioxo-3-piperidyl)-1-methyl-indazol-6-yl]piperazin-1-yl]methyl]-1-piperidyl]butoxy]-2-methyl-phenyl]pyridine-2-carboxylic acid (22.9 mg, 21.4 μmol, 76.5% yield, 99.1% purity, FA) as a yellow solid.

MS (ESI) m/z: 1015.6 [M+H]⁺.

¹H NMR (400 MHz, DMSO-d₆) δ=10.84 (s, 1H), 8.14 (s, 1H), 8.02 (d, J=8.0 Hz, 1H), 7.78 (d, J=8.0 Hz, 1H), 7.62 (d, J=7.2 Hz, 1H), 7.51-7.48 (m, 1H), 7.47-7.40 (m, 3H), 7.39-7.30 (m, 2H), 7.13-7.07 (m, 1H), 6.96-6.87 (m, 3H), 6.83 (s, 1H), 6.65 (d, J=7.6 Hz, 1H), 4.97 (s, 2H), 4.28-4.22 (m, 1H), 4.02 (s, 2H), 3.94-3.90 (m, 2H), 3.88 (s, 3H), 3.21 (d, J=0.8 Hz, 4H), 3.04-3.00 (m, 3H), 2.77-2.70 (m, 2H), 2.65-2.57 (m, 4H), 2.54 (s, 2H), 2.31-2.26 (m, 1H), 2.23-2.11 (m, 4H), 1.92 (s, 3H), 1.86-1.61 (m, 9H), 1.30-1.19 (m, 2H).

### Example 59. Preparation of Compound 239a

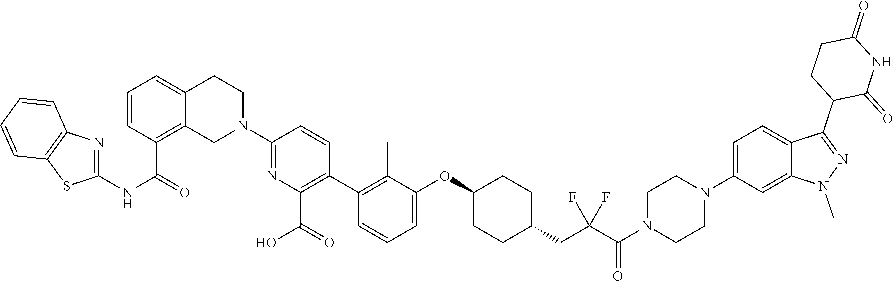 [SMILES: Cc1c(O[C@H]2CC[C@H](CC(F)(F)C(=O)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-6-yl)piperazin-1-yl)-2,2-difluoro-3-oxopropyl)cyclohexyl)oxy)-2-methylphenyl)picolinic acid

### Step A. Procedure for Preparation of methyl 4-(3-bromo-2-methyl-phenoxy) cyclohexanecarboxylate

To a solution of 3-bromo-2-methyl-phenol (30 g, 160.40 mmol, 1 equiv.) in toluene (300 mL) was added methyl 4-hydroxycyclohexanecarboxylate (27.91 g, 176.44 mmol, 1.1 equiv.) and 2-(tributyl-λ⁵-phosphanylidene) acetonitrile (46.46 g, 192.48 mmol, 1.2 equiv.) at 25° C. The reaction mixture was degassed and purged with N₂ three times, and then the mixture was stirred at 120° C. for 16 hours under N₂ atmosphere. The reaction mixture was filtered and concentrated under reduced pressure to give a residue. The residue was purified by column chromatography (SiO₂, petroleum ether/ethyl acetate=1/0 to 10/1) to give methyl 4-(3-bromo-2-methyl-phenoxy) cyclohexanecarboxylate (49 g, 80.5 mmol, 50.2% yield, 53.8% purity) as a colorless oil.

### Step B. Procedure for Preparation of [4-(3-bromo-2-methyl-phenoxy) cyclohexyl]methanol

To a solution of methyl 4-(3-bromo-2-methyl-phenoxy) cyclohexanecarboxylate (49 g, 80.57 mmol, 53.8% purity, 1 equiv.) in THE (500 mL) was added LiAlH₄ (3.97 g, 104.73 mmol, 1.3 equiv.) dropwise at 0° C. under N₂. The reaction mixture was stirred under N₂ at 0° C. for 2 hours. The reaction mixture was quenched with Na₂SO₄·10 H₂O (2 g) and slowly warmed to 20° C. The mixture was filtered and washed with DCM (500 mL). Then the organic layers were concentrated under reduced pressure to give a residue. The residue was purified by column chromatography (SiO₂, petroleum ether/ethyl acetate=20/1 to 5/1) to give [4-(3-bromo-2-methyl-phenoxy) cyclohexyl]methanol (15.7 g, 52.4 mmol, 65.1% yield) as a yellow oil.

¹H NMR (400 MHz, CDCl₃-d) δ=7.14 (d, J=8.0 Hz, 1H), 6.98 (t, J=8.0 Hz, 1H), 6.82 (d, J=8.4 Hz, 1H), 4.12-4.07 (m, 1H), 3.51 (d, J=6.4 Hz, 2H), 2.31 (s, 3H), 2.23-2.14 (m, 2H), 1.96-1.88 (m, 2H), 1.63-1.54 (m, 1H), 1.50-1.46 (m, 2H), 1.16-1.05 (m, 2H)

### Step C. Procedure for Preparation of 4-(3-bromo-2-methyl-phenoxy) cyclohexanecarbaldehyde

A solution of DMSO (16.40 g, 209.89 mmol, 16.40 mL, 4 equiv.) in DCM (10 mL) was added dropwise to a solution of oxalyl dichloride (13.32 g, 104.95 mmol, 9.19 mL, 2 equiv.) in DCM (130 mL) at −70° C. under N₂ atmosphere. The mixture was stirred at −70° C. for 30 minutes. After which time [4-(3-bromo-2-methyl-phenoxy) cyclohexyl]methanol (15.7 g, 52.47 mmol, 1 equiv.) in DCM (10 mL) was added dropwise at −70° C. The solution was stirred for 30 minutes at −70° C. Then TEA (31.86 g, 314.84 mmol, 43.82 mL, 6 equiv.) was added into the solution. The solution was stirred at 25° C. for 1 hour under N₂ atmosphere. The reaction mixture was diluted with water (20 mL) and extracted with DCM (20 mL×3). The combined organic layers were washed with brine (10 mL×2), dried over Na₂SO₄, filtered, and concentrated under reduced pressure to give 4-(3-bromo-2-methyl-phenoxy) cyclohexanecarbaldehyde (22 g, crude) as a yellow oil.

### Step D. Procedure for Preparation of ethyl 3-[4-(3-bromo-2-methyl-phenoxy) cyclohexyl]-2, 2-difluoro-3-hydroxy-propanoate

To a mixture of Zn (2.50 g, 38.28 mmol, 2.5 equiv.) in THE (5 mL) was added 1, 2-dibromoethane (287.62 mg, 1.53 mmol, 115.51 μL, 0.1 equiv.), ethyl 2-bromo-2, 2-difluoro-acetate (4.35 g, 21.43 mmol, 2.75 mL, 1.4 equiv.) at 60° C. The reaction mixture was degassed and purged with N₂ three times, and then the mixture was stirred at 60° C. for 0.5 hours under N₂ atmosphere. Then a solution of 4-(3-bromo-2-methyl-phenoxy) cyclohexanecarbaldehyde (1.5 g, 5.05 mmol, 0.33 equiv.) in THE (1 mL) was added dropwise at 60° C. The mixture was stirred at 60° C. for 16 hours. The mixture was cooled to 20° C. and then filtered. The filtrate was quenched by 1N HCl (100 mL), and then extracted with EtOAc (100 mL×3). The combined organic layers were washed with brine saturated NaHCO₃ (300 mL) and brine (100 mL×1), dried over Na₂SO₄, filtered and concentrated under reduced pressure to give a residue. The residue was purified by flash silica gel chromatography (Eluent of 0˜10% ethyl acetate/petroleum ether) to give ethyl 3-[4-(3-bromo-2-methyl-phenoxy) cyclohexyl]-2, 2-difluoro-3-hydroxy-propanoate (670 mg, 1.5 mmol, 10.3% yield) as a colorless oil.

¹H NMR (400 MHz, CDCl₃-d) δ=7.15 (d, J=8.0 Hz, 1H), 6.98 (t, J=8.0 Hz, 1H), 6.81 (d, J=8.0 Hz, 1H), 4.38 (q, J=7.2 Hz, 2H), 4.15-4.07 (m, 1H), 3.95-3.85 (m, 1H), 2.30 (s, 3H), 2.25-2.17 (m, 2H), 2.13-2.05 (m, 2H), 1.94-1.87 (m, 1H), 1.82-1.73 (m, 1H), 1.52-1.43 (m, 3H), 1.39 (t, J=7.2 Hz, 3H)

### Step E. Procedure for Preparation of ethyl 3-[4-(3-bromo-2-methyl-phenoxy)cyclohexyl]-2,2-difluoro-3-methylsulfanylcarbothioyloxy-propanoate

To a solution of ethyl 3-[4-(3-bromo-2-methyl-phenoxy) cyclohexyl]-2, 2-difluoro-3-hydroxy-propanoate (670 mg, 1.59 mmol, 1 equiv.) in DMF (8.5 mL) was added DBU (968.50 mg, 6.36 mmol, 958.91 μL, 4 equiv.) and CS₂ (1.21 g, 15.90 mmol, 961.07 μL, 10 equiv.) at 20° C. The mixture was stirred at 20° C. for 1 hour under N₂ atmosphere. Then Mel (2.26 g, 15.90 mmol, 990.11 μL, 10 equiv.) was added dropwise at 20° C. and the mixture was stirred at 20° C. for 1 hour under N₂ atmosphere. The mixture was diluted with water 10 mL and extracted with EtOAc (20 mL×3). The combined organic layers were washed with brine (10 mL×2), dried over Na₂SO₄, filtered, and concentrated under reduced pressure to give a residue. The residue was purified by flash silica gel chromatography (Eluent of 0˜ 10% ethyl acetate/petroleum ether) to give ethyl 3-[4-(3-bromo-2-methyl-phenoxy)cyclohexyl]-2,2-difluoro-3-methylsulfanylcarbothioyloxy-propanoate (764 mg, 1.4 mmol, 93.9% yield) as a colorless oil.

¹H NMR (400 MHz, CDCl₃-d) δ=7.15 (d, J=7.6 Hz, 1H), 6.97 (t, J=8.0 Hz, 1H), 6.79 (d, J=8.4 Hz, 1H), 6.31-6.22 (m, 1H), 4.30 (q, J=7.2 Hz, 2H), 4.15-4.04 (m, 1H), 2.63-2.56 (m, 3H), 2.29 (s, 3H), 2.23-2.17 (m, 2H), 2.16-2.08 (m, 1H), 2.04-1.95 (m, 2H), 1.52-1.37 (m, 4H), 1.34 (t, J=7.2 Hz, 3H)

### Step F. Procedure for Preparation of ethyl 3-[4-(3-bromo-2-methyl-phenoxy) cyclohexyl]-2, 2-difluoro-propanoate

A solution of ethyl 3-[4-(3-bromo-2-methyl-phenoxy) cyclohexyl]-2, 2-difluoro-3-methylsulfanyl carbothioyloxy-propanoate (764 mg, 1.49 mmol, 1 equiv.), 2-tert-butylperoxy-2-methyl-propane (240.28 mg, 1.64 mmol, 302.62 μL, 1.1 equiv.) and phenylphosphonoylbenzene (664.48 mg, 3.29 mmol, 2.2 equiv.) in dioxane (8 mL) was degassed and purged with N₂ three times, and then the mixture was stirred at 100° C. for 24 hours under N₂ atmosphere. The reaction mixture was filtered and concentrated under reduced pressure to give a residue. The residue was purified by flash silica gel chromatography (Eluent of 0˜10% ethyl acetate/petroleum ether) to give ethyl 3-[4-(3-bromo-2-methyl-phenoxy) cyclohexyl]-2, 2-difluoro-propanoate (580 mg, 1.4 mmol, 95.8% yield) as a colorless oil.

¹H NMR (400 MHz, CDCl₃-d) δ=7.15 (d, J=7.6 Hz, 1H), 6.97 (t, J=8.0 Hz, 1H), 6.80 (d, J=8.0 Hz, 1H), 4.34 (q, J=7.2 Hz, 2H), 4.14-4.05 (m, 1H), 2.30 (s, 3H), 2.18-2.10 (m, 2H), 2.08-1.98 (m, 2H), 1.95 (d, J=12.8 Hz, 2H), 1.77-1.64 (m, 1H), 1.54-1.45 (m, 2H), 1.37 (t, J=7.2 Hz, 3H), 1.23-1.10 (m, 2H)

### Step G. Procedure for Preparation of 3-[4-(3-bromo-2-methyl-phenoxy) cyclohexyl]-2, 2-difluoro-propanoic acid

To a solution of ethyl 3-[4-(3-bromo-2-methyl-phenoxy) cyclohexyl]-2, 2-difluoro-propanoate (550 mg, 1.36 mmol, 1 equiv.) in THE (5 mL) and H₂O (0.5 mL) was added LiOH·H₂O (170.85 mg, 4.07 mmol, 3 equiv.). The mixture was stirred at 25° C. for 16 hours. The reaction mixture was filtered and concentrated under reduced pressure to give a residue. The residue was treated with water (4 mL). The pH was adjusted to around 3 by progressively adding diluted HCl. The mixture was filtered to give 3-[4-(3-bromo-2-methyl-phenoxy) cyclohexyl]-2, 2-difluoro-propanoic acid (510 mg, 1.30 mmol, 95.94% yield, 96.3% purity) as a white solid.

¹H NMR (400 MHz, CDCl₃-d) δ=7.09 (d, J=8.0 Hz, 1H), 6.86 (t, J=8.0 Hz, 1H), 6.68 (d, J=8.4 Hz, 1H), 4.02-3.90 (m, 1H), 2.22 (s, 3H), 2.05-1.98 (m, 2H), 1.96-1.91 (m, 2H), 1.87-1.84 (m, 2H), 1.71-1.60 (m, 1H), 1.43-1.34 (m, 2H), 1.10-0.96 (m, 2H)

### Step H. Procedure for Preparation of 3-[6-[4-[3-[4-(3-bromo-2-methyl-phenoxy) cyclohexyl]-2, 2-difluoro-propanoyl]piperazin-1-yl]-1-methyl-indazol-3-yl]piperidine-2, 6-dione

To a solution of 3-[4-(3-bromo-2-methyl-phenoxy) cyclohexyl]-2, 2-difluoro-propanoic acid (80 mg, 212.08 μmol, 1 equiv.) in DMF (1 mL) was added HATU (104.83 mg, 275.70 μmol, 1.3 equiv.), DIPEA (82.23 mg, 636.23 μmol, 110.82 μL, 3 equiv.), and 3-(1-methyl-6-piperazin-1-yl-indazol-3-yl) piperidine-2, 6-dione (90.26 mg, 248.07 μmol, 1.17 equiv., HCl) at 25° C. The reaction mixture was stirred at 40° C. for 16 hours. The reaction mixture was diluted with water (5 mL). The mixture was triturated with water (5 mL) at 25° C. for 10 min. The mixture was filtered to give 3-[6-[4-[3-[4-(3-bromo-2-methyl-phenoxy) cyclohexyl]-2, 2-difluoro-propanoyl]piperazin-1-yl]-1-methyl-indazol-3-yl]piperidine-2, 6-dione (110 mg, 155.2 μmol, 73.2% yield, 96.9% purity) as a yellow solid.

MS (ESI) m/z: 688.6 [M+H]⁺.

### Step I. Procedure for preparation of tert-butyl 6-[8-(1, 3-benzothiazol-2-ylcarbamoyl)-3, 4-dihydro-1H-isoquinolin-2-yl]-3-[3-[4-[3-[4-[3-(2, 6-dioxo-3-piperidyl)-1-methyl-indazol-6-yl]piperazin-1-yl]-2, 2-difluoro-3-oxo-propyl]cyclohexoxy]-2-methyl-phenyl]pyridine-2-carboxylate

3-[6-[4-[3-[4-(3-bromo-2-methyl-phenoxy) cyclohexyl]-2, 2-difluoro-propanoyl]piperazin-1-yl]-1-methyl-indazol-3-yl]piperidine-2, 6-dione (110 mg, 160.21 μmol, 1 equiv.), tert-butyl 6-[8-(1, 3-benzothiazol-2-ylcarbamoyl)-3, 4-dihydro-1H-isoquinolin-2-yl]-3-(4, 4, 5, 5-tetramethyl-1, 3, 2-dioxaborolan-2-yl) pyridine-2-carboxylate (127.58 mg, 208.28 μmol, 1.3 equiv.), KF (27.92 mg, 480.64 μmol, 11.26 μL, 3 equiv.), and [2-(2-aminophenyl) phenyl]palladium (1+); bis (1-adamantyl)-butyl-phosphane; methanesulfonate (23.34 mg, 32.04 μmol, 0.2 equiv.) were taken up into a microwave tube in dioxane (1 mL) and H₂O (0.1 mL). The sealed tube was heated at 100° C. for 60 min under microwave. The reaction mixture was filtered and concentrated under reduced pressure to give a residue. The residue was purified by prep-TLC (SiO₂, DCM:MeOH=10:1) to give tert-butyl 6-[8-(1, 3-benzothiazol-2-ylcarbamoyl)-3, 4-dihydro-1H-isoquinolin-2-yl]-3-[3-[4-[3-[4-[3-(2, 6-dioxo-3-piperidyl)-1-methyl-indazol-6-yl]piperazin-1-yl]-2, 2-difluoro-3-oxo-propyl]cyclohexoxy]-2-methyl-phenyl]pyridine-2-carboxylate (95 mg, 75.8 μmol, 47.1% yield, 86.9% purity) as a white solid.

MS (ESI) m/z: 1092.9 [M+H]⁺.

### Step J. Procedure for Preparation of 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-6-yl)piperazin-1-yl)-2,2-difluoro-3-oxopropyl)cyclohexyl)oxy)-2-methylphenyl)picolinic acid

solution of tert-butyl 6-[8-(1, 3-benzothiazol-2-ylcarbamoyl)-3, 4-dihydro-1H-isoquinolin-2-yl]-3-[3-[4-[3-[4-[3-(2, 6-dioxo-3-piperidyl)-1-methyl-indazol-6-yl]piperazin-1-yl]-2, 2-difluoro-3-oxo-propyl]cyclohexoxy]-2-methyl-phenyl]pyridine-2-carboxylate (95 mg, 86.98 μmol, 1 equiv.) in DCM (0.5 mL) and TFA (0.5 mL) was stirred at 25° C. for 40 hours. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-6-yl)piperazin-1-yl)-2,2-difluoro-3-oxopropyl)cyclohexyl)oxy)-2-methylphenyl)picolinic acid (37.8 mg, 35.6 μmol, 40.9% yield, 97.4% purity) as an off-white solid.

MS (ESI) m/z: 1036.9 [M+H]⁺.

¹H NMR (400 MHz, DMSO-d₆) δ=12.95-12.29 (m, 2H), 10.85 (s, 1H), 8.03 (d, J=8.0 Hz, 1H), 7.79 (d, J=8.0 Hz, 1H), 7.62 (d, J=7.6 Hz, 1H), 7.54 (d, J=8.8 Hz, 1H), 7.49-7.42 (m, 3H), 7.40-7.32 (m, 2H), 7.10-7.04 (m, 1H), 6.98-6.89 (m, 4H), 6.61 (d, J=7.6 Hz, 1H), 4.97 (s, 2H), 4.27 (dd, J=5.2, 9.2 Hz, 1H), 4.23-4.14 (m, 1H), 3.90 (s, 5H), 3.85-3.79 (m, 2H), 3.76-3.69 (m, 2H), 3.28-3.28 (m, 2H), 3.02 (t, J=5.2 Hz, 2H), 2.68-2.58 (m, 2H), 2.35-2.27 (m, 2H), 2.21-1.99 (m, 6H), 1.86 (s, 5H), 1.73-1.64 (m, 1H), 1.46-1.33 (m, 2H), 1.30-1.20 (m, 2H)

### Example 60. Preparation of Compound 240b

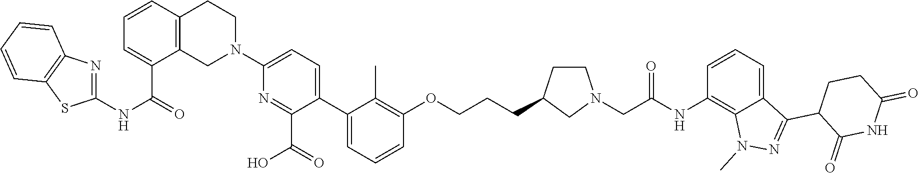 [SMILES: Cc1c(OCCC[C@H]2CCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-((3S)-1-(2-((3-(2,6-dioxopiperidin-3-yl)-1-methyl-1H-indazol-7-yl)amino)-2-oxoethyl)pyrrolidin-3-yl)propoxy)-2-methylphenyl)picolinic acid

### Step A. Procedure for Preparation of (S)-tert-butyl 6-(8-(benzo[d]thiazol-2-ylcarbamoyl)-3,4-dihydroisoquinolin-2(1H)-yl)-3-(3-(3-(1-(2-methoxy-2-oxoethyl)pyrrolidin-3-yl)propoxy)-2-methylphenyl)picolinate

(S)-ethyl 2-(3-(3-(3-bromo-2-methylphenoxy)propyl)pyrrolidin-1-yl)acetate (126 mg, 327.86 μmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (220.91 mg, 360.65 μmol, 1.1 equiv.), Ad₂nBuP Pd G₃ (47.75 mg, 65.57 μmol, 0.2 equiv.), and K₂CO₃ (1.5 M, 327.86 μL, 1.5 equiv.) in dioxane (3 mL) and were taken up into a microwave tube in dioxane (3 mL). The sealed tube was heated at 100° C. for 2 hours under microwave. The reaction mixture was partitioned between H₂O (15 mL) and ethyl acetate (20 mL×3). The organic phase was separated, washed with aqueous NaCl (15 mL×3), dried over Na₂SO₄, filtered, and concentrated under reduced pressure to give a residue. The residue was purified by prep-TLC (SiO₂, petroleum ether:ethyl acetate=0:1) to give (S)-tert-butyl 6-(8-(benzo[d]thiazol-2-ylcarbamoyl)-3,4-dihydroisoquinolin-2(1H)-yl)-3-(3-(3-(1-(2-methoxy-2-oxoethyl)pyrrolidin-3-yl)propoxy)-2-methylphenyl)picolinate (220 mg, 238.1 μmol, 72.6% yield, 85.5% purity) as a yellow solid

MS (ESI) m/z: 790.5 [M+H]⁺.

### Step B. Procedure for Preparation of (S)-2-(3-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)pyrrolidin-1-yl)acetic acid

A mixture of (S)-tert-butyl 6-(8-(benzo[d]thiazol-2-ylcarbamoyl)-3,4-dihydroisoquinolin-2(1H)-yl)-3-(3-(3-(1-(2-methoxy-2-oxoethyl)pyrrolidin-3-yl)propoxy)-2-methylphenyl)picolinate (220 mg, 278.49 μmol, 1 equiv.), and LiOH·H₂O (1 M, 835.46 μL, 3 equiv.) in THE (6 mL) was stirred at 25° C. for 15 hours. The mixture was concentrated and diluted with H₂O (2 mL), then the pH of the mixture was adjusted to 3 with 1 M HCl. Then the mixture was filtered and concentrated to give (S)-2-(3-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)pyrrolidin-1-yl)acetic acid (130 mg, 162.2 μmol, 58.2% yield, 95.1% purity) as a white solid.

MS (ESI) m/z: 762.1 [M+H]⁺.

### Step C. Procedure for Preparation of tert-butyl 6-(8-(benzo[d]thiazol-2-ylcarbamoyl)-3,4-dihydroisoquinolin-2(1H)-yl)-3-(3-(3-((3S)-1-(2-((3-(2,6-dioxopiperidin-3-yl)-1-methyl-11H-indazol-7-yl)amino)-2-oxoethyl)pyrrolidin-3-yl)propoxy)-2-methylphenyl)picolinate

A mixture of (S)-2-(3-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)pyrrolidin-1-yl)acetic acid (130 mg, 170.62 μmol, 1 equiv.), 3-(7-amino-1-methyl-1H-indazol-3-yl)piperidine-2,6-dione (52.88 mg, 204.74 μmol, 1.2 equiv.) in pyridine (2 mL) was added EDCI (49.06 mg, 255.93 μmol, 1.5 equiv.), and then the mixture was stirred at 25° C. for 3 hours. The reaction mixture was partitioned between H₂O (10 mL) and DCM (10 mL×3). The organic phase was separated, washed with aqueous NaCl (10 mL×2), dried over Na₂SO₄, filtered, and concentrated under reduced pressure to give tert-butyl 6-(8-(benzo[d]thiazol-2-ylcarbamoyl)-3,4-dihydroisoquinolin-2(1H)-yl)-3-(3-(3-((3S)-1-(2-((3-(2,6-dioxopiperidin-3-yl)-1-methyl-1H-indazol-7-yl)amino)-2-oxoethyl)pyrrolidin-3-yl)propoxy)-2-methylphenyl)picolinate (130 mg, crude) as a yellow solid.

MS (ESI) m/z: 1002.4 [M+H]⁺.

### Step D. Procedure for Preparation of 6-(8-(benzo[d]thiazol-2-ylcarbamoyl)-3,4-dihydroisoquinolin-2(1H)-yl)-3-(3-(3-((3S)-1-(2-((3-(2,6-dioxopiperidin-3-yl)-1-methyl-11H-indazol-7-yl)amino)-2-oxoethyl)pyrrolidin-3-yl)propoxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(3-(3-((3S)-1-(2-((3-(2,6-dioxopiperidin-3-yl)-1-methyl-1H-indazol-7-yl)amino)-2-oxoethyl)pyrrolidin-3-yl)propoxy)-2-methylphenyl)picolinate (126.15 mg, 125.88 μmol, 1 equiv.) in DCM (3 mL) and TFA (1 mL) was stirred at 40° C. for 39 hours. The reaction mixture was concentrated under the reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(3-((3S)-1-(2-((3-(2,6-dioxopiperidin-3-yl)-1-methyl-1H-indazol-7-yl)amino)-2-oxoethyl)pyrrolidin-3-yl)propoxy)-2-methylphenyl)picolinic acid (24.9 mg, 25.3 μmol, 20.1% yield, 96.1% purity) as a yellow solid

MS (ESI) m/z: 946.6 [M+H]⁺.

¹H NMR (400 MHz, DMSO-d₆) δ=10.89 (s, 1H), 9.88 (s, 1H), 8.16 (s, 1H), 8.02 (d, J=7.2 Hz, 1H), 7.78 (d, J=8.0 Hz, 1H), 7.62 (d, J=8.0 Hz, 1H), 7.56 (d, J=8.0 Hz, 1H), 7.49-7.41 (m, 3H), 7.39-7.32 (m, 2H), 7.28 (d, J=7.2 Hz, 1H), 7.07 (q, J=8.0 Hz, 2H), 6.96-6.92 (m, 1H), 6.88 (d, J=8.0 Hz, 1H), 6.63 (d, J=7.6 Hz, 1H), 4.97 (s, 2H), 4.36 (dd, J=5.2, 10 Hz, 1H), 4.08 (s, 3H), 3.98 (s, 2H), 3.91 (t, J=5.6 Hz, 2H), 3.02 (s, 2H), 2.99-2.93 (m, 2H), 2.86-2.76 (m, 2H), 2.62-2.58 (m, 2H), 2.26-2.11 (m, 3H), 2.01 (dd, J=1.6, 5.6 Hz, 2H), 1.90 (s, 3H), 1.78-1.71 (m, 2H), 1.55-1.40 (m, 4H)

### Example 61. Preparation of Compound 241

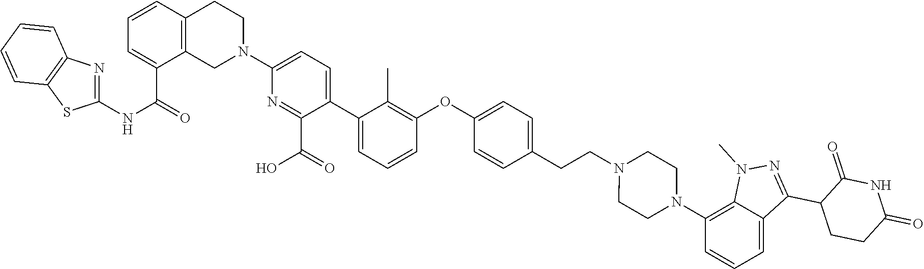 [SMILES: Cc1c(Oc2ccc(CCN3CCN(c4cccc5c(C6CCC(=O)NC6=O)nn(C)c45)CC3)cc2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[4-[2-[4-[3-(2,6-dioxo-3-piperidyl)-1-methyl-indazol-7-yl]piperazin-1-yl]ethyl]phenoxy]-2-methyl-phenyl]pyridine-2-carboxylic acid

### Step A. Procedure for Preparation of 1-bromo-3-(4-(2-chloroethyl)phenoxy)-2-methylbenzene

To a solution of 2-(4-(3-bromo-2-methylphenoxy)phenyl)ethan-1-ol (100 mg, 325.54 μmol, 1 equiv.) in SOCl₂ (3 mL). The mixture was stirred at 80° C. for 2 hours. The reaction mixture was concentrated under reduced pressure to give a product 1-bromo-3-(4-(2-chloroethyl)phenoxy)-2-methylbenzene (100 mg, crude) was obtained as a yellow oil.

### Step B. Procedure for Preparation of 3-(7-(4-(4-(3-bromo-2-methylphenoxy)phenethyl)piperazin-1-yl)-1-methyl-1H-indazol-3-yl)piperidine-2,6-dione

To a solution of 1-bromo-3-(4-(2-chloroethyl)phenoxy)-2-methylbenzene (100 mg, 307.10 μmol, 1 equiv.), 3-(1-methyl-7-(piperazin-1-yl)-1H-indazol-3-yl)piperidine-2,6-dione (100.54 mg, 307.10 μmol, 1 equiv.) in NMP (1 mL) was added KI (50.98 mg, 307.10 μmol, 1 equiv.) and DIEA (198.45 mg, 1.54 mmol, 267.46 μL, 5 equiv.). The mixture was stirred at 60° C. for 16 hours. The reaction mixture was concentrated under reduced pressure to give a residue. The residue was purified by column chromatography (SiO₂, by prep-TLC (SiO₂, DCM:MeOH=20:1) to give 3-(7-(4-(4-(3-bromo-2-methylphenoxy)phenethyl)piperazin-1-yl)-1-methyl-1H-indazol-3-yl)piperidine-2,6-dione (65 mg, 82.2 μmol, 26.8% yield, 78% purity) as a yellow oil.

MS (ESI) m/z: 618.1 [M+H]⁺.

### Step C. Procedure for Preparation of tert-butyl 6-[8-(1,3-benzothiazol-2-ylcarbamoyl)-3,4-dihydro-1H-isoquinolin-2-yl]-3-[3-[4-[2-[4-[3-(2,6-dioxo-3-piperidyl)-1-methyl-indazol-7-yl]piperazin-1-yl]ethyl]phenoxy]-2-methyl-phenyl]pyridine-2-carboxylate

3-(7-(4-(4-(3-bromo-2-methylphenoxy)phenethyl)piperazin-1-yl)-1-methyl-1H-indazol-3-yl)piperidine-2,6-dione (60 mg, 97.32 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (65.57 mg, 107.05 μmol, 1.1 equiv.) and [2-(2-aminophenyl)phenyl]palladium(1+); bis(1-adamantyl)-butyl-phosphane; methanesulfonate (7.09 mg, 9.73 μmol, 0.1 equiv.), and KF (16.96 mg, 291.95 μmol, 6.84 μL, 3 equiv.) were taken up into a microwave tube in dioxane (2 mL) and H₂O (0.2 mL). The sealed tube was heated at 100° C. for 1 hour under microwave. The reaction mixture was concentrated under reduced pressure to give a residue. The residue was purified by column chromatography (SiO₂, by prep-TLC (SiO₂, DCM:MeOH=20:1) to give tert-butyl 6-[8-(1,3-benzothiazol-2-ylcarbamoyl)-3,4-dihydro-1H-isoquinolin-2-yl]-3-[3-[4-[2-[4-[3-(2,6-dioxo-3-piperidyl)-1-methyl-indazol-7-yl]piperazin-1-yl]ethyl]phenoxy]-2-methyl-phenyl]pyridine-2-carboxylate (55 mg, 45.5 μmol, 46.7% yield, 84.6% purity) as a white oil.

MS (ESI) m/z: 1022.3 [M+H]⁺.

### Step D. Procedure for Preparation of 6-[8-(1,3-benzothiazol-2-ylcarbamoyl)-3,4-dihydro-1H-isoquinolin-2-yl]-3-[3-[4-[2-[4-[3-(2,6-dioxo-3-piperidyl)-1-methyl-indazol-7-yl]piperazin-1-yl]ethyl]phenoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[4-[2-[4-[3-(2,6-dioxo-3-piperidyl)-1-methyl-indazol-7-yl]piperazin-1-yl]ethyl]phenoxy]-2-methyl-phenyl]pyridine-2-carboxylate (50 mg, 48.91 μmol, 1 equiv.) in DCM (0.5 mL) was added TFA (770.00 mg, 6.75 mmol, 0.5 mL, 138.06 equiv.). The mixture was stirred at 25° C. for 3 hours. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[4-[2-[4-[3-(2,6-dioxo-3-piperidyl)-1-methyl-indazol-7-yl]piperazin-1-yl]ethyl]phenoxy]-2-methyl-phenyl]pyridine-2-carboxylic acid (4.1 mg, 4.1 μmol, 8.4% yield, 95% purity) as a yellow solid.

MS (ESI) m/z: 966.6[M+H]⁺.

¹H NMR (400 MHz, DMSO-d₆) δ=10.94-10.82 (m, 1H), 8.29 (s, 1H), 8.00 (d, J=7.6 Hz, 1H), 7.76 (d, J=8.0 Hz, 1H), 7.64 (d, J=6.8 Hz, 1H), 7.43 (d, J=6.4 Hz, 2H), 7.39-7.36 (m, 1H), 7.36-7.29 (m, 2H), 7.22 (d, J=8.4 Hz, 2H), 7.16-7.10 (m, 1H), 7.06-7.00 (m, 2H), 6.94 (d, J=8.0 Hz, 1H), 6.87-6.78 (m, 4H), 4.99 (s, 2H), 4.34 (dd, J=5.2, 9.6 Hz, 1H), 4.25 (s, 3H), 3.91 (t, J=5.6 Hz, 2H), 3.04-2.99 (m, 4H), 2.78-2.74 (m, 2H), 2.67 (d, J=1.6 Hz, 1H), 2.63-2.57 (m, 4H), 2.52 (s, 2H), 2.43 (s, 1H), 2.36-2.30 (m, 2H), 2.20-2.14 (m, 1H), 1.94 (s, 3H)

### Example 62. Preparation of Compound 245a

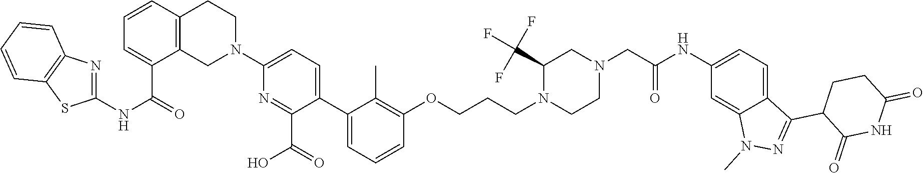 [SMILES: Cc1c(OCCCN2CCN(CC(=O)Nc3ccc4c(C5CCC(=O)NC5=O)nn(C)c4c3)C[C@@H]2C(F)(F)F)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-((2R)-4-(2-((3-(2,6-dioxopiperidin-3-yl)-1-methyl-1H-indazol-6-yl)amino)-2-oxoethyl)-2-(trifluoromethyl)piperazin-1-yl)propoxy)-2-methylphenyl)picolinic acid

### Step A. Procedure for Preparation of tert-butyl (R)-4-allyl-3-(trifluoromethyl)piperazine-1-carboxylate

To a solution of tert-butyl (R)-3-(trifluoromethyl)piperazine-1-carboxylate (500 mg, 1.97 mmol, 1.0 equiv.) in CH₃CN (7 mL) was added K₂CO₃ (815.38 mg, 5.90 mmol, 3.0 equiv.), KI (32.65 mg, 196.66 μmol, 0.1 equiv.), and 3-bromoprop-1-ene (237.91 mg, 1.97 mmol, 1.0 equiv.). The mixture was stirred at 80° C. for 12 hours. The reaction mixture was filtered and concentrated under reduced pressure to give a residue. The residue was purified by column chromatography (SiO₂, petroleum ether/ethyl acetate=1/0 to 50/1) to give tert-butyl (R)-4-allyl-3-(trifluoromethyl)piperazine-1-carboxylate (400 mg, crude) as a yellow oil.

¹H NMR (400 MHz, CDCl₃) δ=5.95-5.64 (m, 1H), 5.41-5.07 (m, 2H), 4.23-3.55 (m, 2H), 3.50-3.28 (m, 3H), 3.24-3.08 (m, 2H), 2.91 (t, J=10.8 Hz, 1H), 2.65 (d, J=10.8 Hz, 1H), 1.46 (s, 9H).

### Step B. Procedure for Preparation of tert-butyl (R)-4-(3-hydroxypropyl)-3-(trifluoromethyl) piperazine-1-carboxylate

To a mixture of tert-butyl (R)-4-allyl-3-(trifluoromethyl)piperazine-1-carboxylate (400 mg, 1.36 mmol, 1 equiv.) in THE (5 mL) was added BH₃·THF (1 M, 4.08 mL, 3.0 equiv.). The mixture was degassed and purged with N₂ three times, and then the mixture was stirred at 0° C. for 2 hours. Afterwards, NaOH (2 M, 1.36 mL, 2.0 equiv.) was added slowly, and the reaction was stirred for 2 hours under N₂ atmosphere. Then H₂O₂ (924.58 mg, 8.15 mmol, 783.55 μL, 30% purity, 6.0 equiv.) was added to the mixture at 0° C., and the mixture was stirred at 25° C. for 3 hours. The reaction mixture was quenched by addition Na₂SO₃ (saturated solution, 80 mL) at 25° C., and the pH of the solution was adjusted to 5-7. The resulting mixture was extracted with DCM (20 mL×3). The combined organic layers were washed with brine (10 mL×3), dried over Na₂SO₄, and then concentrated (below 30° C.) under reduced pressure to give a residue. The residue was purified by column chromatography (SiO₂, petroleum ether/ethyl acetate 1/0 to 2/1) to give tert-butyl (R)-4-(3-hydroxypropyl)-3-(trifluoromethyl) piperazine-1-carboxylate (180 mg, crude) as a colorless oil.

¹H NMR (400 MHz, CDCl₃) δ=4.25-3.88 (m, 2H), 3.78 (t, J=5.6 Hz, 2H), 3.34 (s, 1H), 3.26-3.05 (m, 2H), 3.01-2.93 (m, 3H), 2.77-2.64 (m, 1H), 1.80-1.74 (m, 2H), 1.46 (s, 9H).

### Step C. Procedure for Preparation of tert-butyl (R)-4-(3-(3-bromo-2-methylphenoxy)propyl)-3-(trifluoromethyl)piperazine-1-carboxylate

A mixture of tert-butyl (R)-4-(3-hydroxypropyl)-3-(trifluoromethyl)piperazine-1-carboxylate (180 mg, 576.32 μmol, 1.0 equiv.), 3-bromo-2-methylphenol (107.79 mg, 576.32 μmol, 1.0 equiv.), and 2-(tributyl-λ5-phosphanylidene)acetonitrile (166.92 mg, 691.58 μmol, 1.2 equiv.) in toluene (4 mL) was degassed and purged with N₂ three times, and then the mixture was stirred at 120° C. for 4 hours under N₂ atmosphere. The reaction mixture was filtered and concentrated under reduced pressure to give a residue. The residue was purified by prep-HPLC to give tert-butyl (R)-4-(3-(3-bromo-2-methylphenoxy)propyl)-3-(trifluoromethyl)piperazine-1-carboxylate (110 mg, crude) as a yellow oil.

MS (ESI) m/z: 481.1 [M+H]⁺.

¹H NMR (400 MHz, CDCl₃) δ=7.16 (d, J=8.0 Hz, 1H), 7.00 (t, J=8.0 Hz, 1H), 6.77 (d, J=8.0 Hz, 1H), 4.13-3.91 (m, 3H), 3.43-3.23 (m, 1H), 3.17-3.04 (m, 2H), 3.02-2.90 (m, 3H), 2.66 (d, J=10.8 Hz, 1H), 2.31 (s, 3H), 2.03-1.93 (m, 2H), 1.58 (s, 1H), 1.46 (s, 9H).

### Step D. Procedure for Preparation of (R)-1-(3-(3-bromo-2-methylphenoxy)propyl)-2-(trifluoromethyl)piperazine

A solution of tert-butyl (R)-4-(3-(3-bromo-2-methylphenoxy)propyl)-3-(trifluoromethyl)piperazine-1-carboxylate (110 mg, 228.53 μmol, 1.0 equiv.) in HCl/dioxane (4 M, 1 mL, 17.50 equiv.) was stirred at 25° C. for 2 hours. The reaction mixture was filtered and concentrated under reduced pressure to give (R)-1-(3-(3-bromo-2-methylphenoxy)propyl)-2-(trifluoromethyl)piperazine (110 mg, crude) as a white solid.

MS (ESI) m/z: 381.0 [M+H]⁺.

### Step E. Procedure for Preparation of ethyl (R)-2-(4-(3-(3-bromo-2-methylphenoxy)propyl)-3-(trifluoromethyl)piperazin-1-yl)acetate

To a solution of (R)-1-(3-(3-bromo-2-methylphenoxy)propyl)-2-(trifluoromethyl)piperazine (110 mg, 288.54 μmol, 1.0 equiv.) in CH₃CN (2 mL) was added K₂CO₃ (199.40 mg, 1.44 mmol, 5.0 equiv.), KI (4.79 mg, 28.85 μmol, 0.1 equiv.), and ethyl 2-bromoacetate (48.19 mg, 288.54 μmol, 31.91 μL, 1.0 equiv.). The mixture was stirred at 60° C. for 4 hours. The reaction mixture was filtered and concentrated under reduced pressure to give a residue. The residue was purified by prep-HPLC to give ethyl (R)-2-(4-(3-(3-bromo-2-methylphenoxy)propyl)-3-(trifluoromethyl)piperazin-1-yl)acetate (120 mg, crude) as a black brown oil.

MS (ESI) m/z: 467.0 [M+H]⁺

### Step F. Procedure for Preparation of tert-butyl (R)-6-(8-(benzo[d]thiazol-2-ylcarbamoyl)-3,4-dihydroisoquinolin-2(1H)-yl)-3-(3-(3-(4-(2-ethoxy-2-oxoethyl)-2-(trifluoromethyl)piperazin-1-yl)propoxy)-2-methylphenyl)picolinate

To a mixture of tert-butyl 6-(8-((3H-indol-2-yl)carbamoyl)-3,4-dihydroisoquinolin-2(1H)-yl)-3-(4,4,5,5-tetramethyl-1,3,2-dioxaborolan-2-yl)picolinate (157.29 mg, 256.78 μmol, 1.0 equiv.) and ethyl (R)-2-(4-(3-(3-bromo-2-methylphenoxy)propyl)-3-(trifluoromethyl)piperazin-1-yl)acetate (120 mg, 256.78 μmol, 1.0 equiv.) in dioxane (3 mL) was added KF (1.5 M, 513.57 μL, 3.0 equiv.) and Ad₂nBuP Pd G₃ (18.70 mg, 25.68 μmol, 0.1 equiv.). After addition, the mixture was degassed and purged with N₂ three times, and then the mixture was stirred at 100° C. for 2 hours under N₂ atmosphere. The reaction mixture was filtered and concentrated under reduced pressure to give a residue. The residue was purified by flash silica gel chromatography (Eluent of 0˜40% ethyl acetate/petroleum ether) to give compound tert-butyl (R)-6-(8-(benzo[d]thiazol-2-ylcarbamoyl)-3,4-dihydroisoquinolin-2(1H)-yl)-3-(3-(3-(4-(2-ethoxy-2-oxoethyl)-2-(trifluoromethyl)piperazin-1-yl)propoxy)-2-methylphenyl)picolinate (200 mg, crude) as a yellow solid.

MS (ESI) m/z: 873.6 [M+H]⁺.

### Step G. Procedure for Preparation of (R)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-3-(trifluoromethyl)piperazin-1-yl)acetic acid

To a solution of tert-butyl (R)-6-(8-(benzo[d]thiazol-2-ylcarbamoyl)-3,4-dihydroisoquinolin-2(1H)-yl)-3-(3-(3-(4-(2-ethoxy-2-oxoethyl)-2-(trifluoromethyl)piperazin-1-yl)propoxy)-2-methylphenyl)picolinate (200 mg, 229.10 μmol, 1.0 equiv.) in THE (2 mL) was added LiOH·H₂O (28.84 mg, 687.29 μmol, 3.0 equiv.) and H₂O (0.6 mL). The mixture was stirred at 25° C. for 12 hours. The reaction mixture was concentrated under reduced pressure to remove THF. The aqueous phase was adjusted to pH=4-5 with 1M HCl. The reaction mixture was filtered and diluted with ethyl acetate. The combined organic layers were dried over Na₂SO₄, filtered, and concentrated under reduced pressure to give (R)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-3-(trifluoromethyl)piperazin-1-yl)acetic acid (180 mg, crude) as a white solid.

MS (ESI) m/z: 845.6 [M+H]⁺.

### Step H. Procedure for Preparation of tert-butyl 6-(8-(benzo[d]thiazol-2-ylcarbamoyl)-3,4-dihydroisoquinolin-2(1H)-yl)-3-(3-(3-((2R)-4-(2-((3-(2,6-dioxopiperidin-3-yl)-1-methyl-11H-indazol-6-yl)amino)-2-oxoethyl)-2-(trifluoromethyl)piperazin-1-yl)propoxy)-2-methylphenyl)picolinate

To a solution of (R)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-3-(trifluoromethyl)piperazin-1-yl)acetic acid (90 mg, 106.52 μmol, 1.0 equiv.), 3-(6-amino-1-methyl-1H-indazol-3-yl)piperidine-2,6-dione (27.51 mg, 106.52 μmol, 1.0 equiv.) in pyridine (1.5 mL) was added EDCI (30.63 mg, 159.77 μmol, 1.5 equiv.). The mixture was stirred at 25° C. for 12 hours. The reaction mixture was diluted with water (2 mL) and extracted with ethyl acetate (2 mL×5). The combined organic layers were washed with brine (5 mL×2), dried over Na₂SO₄, filtered and concentrated under reduced pressure to give tert-butyl 6-(8-(benzo[d]thiazol-2-ylcarbamoyl)-3,4-dihydroisoquinolin-2(1H)-yl)-3-(3-(3-((2R)-4-(2-((3-(2,6-dioxopiperidin-3-yl)-1-methyl-1H-indazol-6-yl)amino)-2-oxoethyl)-2-(trifluoromethyl)piperazin-1-yl)propoxy)-2-methylphenyl)picolinate (100 mg, crude) as a white solid.

MS (ESI) m/z: 1085.4 [M+H]⁺.

### Step L Procedure for preparation of 6-(8-(benzo[d]thiazol-2-ylcarbamoyl)-3,4-dihydroisoquinolin-2(1H)-yl)-3-(3-(3-((2R)-4-(2-((3-(2,6-dioxopiperidin-3-yl)-1-methyl-11H-indazol-6-yl)amino)-2-oxoethyl)-2-(trifluoromethyl)piperazin-1-yl)propoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(3-((2R)-4-(2-((3-(2,6-dioxopiperidin-3-yl)-1-methyl-1H-indazol-6-yl)amino)-2-oxoethyl)-2-(trifluoromethyl)piperazin-1-yl)propoxy)-2-methylphenyl)picolinate (100 mg, 92.15 μmol, 1.0 equiv.) in DCM (1 mL) was added TFA (770.00 mg, 6.75 mmol, 0.5 mL, 73.28 equiv.). The mixture was stirred at 25° C. for 12 hours. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(3-((2R)-4-(2-((3-(2,6-dioxopiperidin-3-yl)-1-methyl-1H-indazol-6-yl)amino)-2-oxoethyl)-2-(trifluoromethyl)piperazin-1-yl)propoxy)-2-methylphenyl)picolinic acid (56.5 mg, 54.5 μmol, 59.1% yield, 99.3% purity) as a white solid.

MS (ESI) m/z: 1029.3 [M+H]⁺.

¹H NMR (400 MHz, DMSO-d₆) δ=12.98-12.33 (m, 2H), 10.88 (s, 1H), 9.96-9.63 (m, 1H), 8.13 (s, 1H), 8.08-7.99 (m, 2H), 7.79 (d, J=8.0 Hz, 1H), 7.69-7.59 (m, 2H), 7.51-7.41 (m, 3H), 7.40-7.30 (m, 2H), 7.14-7.01 (m, 2H), 6.97 (d, J=8.8 Hz, 1H), 6.88 (d, J=8.4 Hz, 1H), 6.63 (d, J=7.6 Hz, 1H), 4.98 (s, 2H), 4.32 (dd, J=5.2, 4.8 Hz, 1H), 4.01 (t, J=6.0 Hz, 2H), 3.95-3.86 (m, 5H), 3.60-3.46 (m, 2H), 3.23-3.15 (m, 1H), 3.03 (t, J=5.6 Hz, 3H), 2.94-2.83 (m, 3H), 2.77-2.57 (m, 6H), 2.40-2.28 (m, 1H), 2.23-2.10 (m, 1H), 1.99-1.82 (m, 5H).

### Example 63. Preparation of Compound 245b

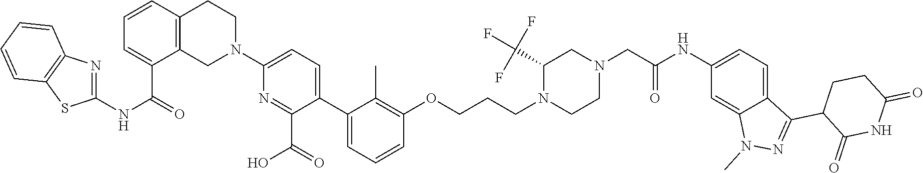 [SMILES: Cc1c(OCCCN2CCN(CC(=O)Nc3ccc4c(C5CCC(=O)NC5=O)nn(C)c4c3)C[C@H]2C(F)(F)F)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-((2S)-4-(2-((3-(2,6-dioxopiperidin-3-yl)-1-methyl-1H-indazol-6-yl)amino)-2-oxoethyl)-2-(trifluoromethyl)piperazin-1-yl)propoxy)-2-methylphenyl)picolinic acid

### Step A. Procedure for Preparation of (S)-tert-butyl 4-allyl-3-(trifluoromethyl)piperazine-1-carboxylate

A mixture of (S)-tert-butyl 3-(trifluoromethyl)piperazine-1-carboxylate (400 mg, 1.57 mmol, 1 equiv.), 3-bromoprop-1-ene (209.36 mg, 1.73 mmol, 1.10 equiv.), KI (26.12 mg, 157.33 μmol, 0.10 equiv.), and K₂CO₃ (652.30 mg, 4.72 mmol, 3.00 equiv.) in CH₃CN (5 mL) was degassed and purged with N₂ three times, and then the mixture was stirred at 80° C. for 6 hours under N₂ atmosphere. The reaction mixture was diluted with water (20 mL) and extracted with EtOAc (25 mL×3). The combined organic layers were washed with brine (15 mL×3), dried over anhydrous sodium sulfate, filtered, and concentrated under reduced pressure to give a residue. The residue was purified by column chromatography (SiO₂, petroleum ether/ethyl acetate 300/1 to 10/1) to give (S)-tert-butyl 4-allyl-3-(trifluoromethyl)piperazine-1-carboxylate (280 mg, 951.3 μmol, 60.4% yield) as a colorless oil.

¹H NMR (400 MHz, CDCl₃) δ=5.91-5.68 (m, 1H), 5.32-5.16 (m, 2H), 4.17-3.84 (m, 2H), 3.44-3.30 (m, 3H), 3.26-3.05 (m, 2H), 2.98-2.86 (m, 1H), 2.68-2.57 (m, 1H), 1.46 (s, 9H)

### Step B. Procedure for Preparation of (S)-tert-butyl 4-(3-hydroxypropyl)-3-(trifluoromethyl)piperazine-1-carboxylate

To a mixture of (S)-tert-butyl 4-allyl-3-(trifluoromethyl)piperazine-1-carboxylate (150 mg, 509.66 μmol, 1.00 equiv.) in THF (1 mL) was added BH₃·THF (1 M, 1.53 mL, 3.00 equiv.). The mixture was degassed and purged with N₂ three times, and then the mixture was stirred at 0° C. for 1.5 hours under N₂ atmosphere. Then NaOH (2 M, 509.66 μL, 2.00 equiv.) was slowly added, and the mixture was stirred for 2 hours under N₂ atmosphere. After stirring, H₂O₂ (346.72 mg, 3.06 mmol, 293.83 μL, 30% purity, 6.00 equiv.) was slowly added into the mixture. The mixture was stirred at 25° C. for 3 hours. The reaction mixture was quenched by addition of Na₂SO₃ (saturated solution, 50 mL) at 25° C. The pH of the solution was adjusted to 5-7, and then extracted with DCM (10 mL×3). The combined organic layers were washed with brine (10 mL×3), dried over Na₂SO₄, and then concentrated (below 30° C.) under reduced pressure to give a residue. The residue was purified by prep-TLC (SiO₂, petroleum ether:ethyl acetate=3:1) to give (S)-tert-butyl 4-(3-hydroxypropyl)-3-(trifluoromethyl)piperazine-1-carboxylate (40 mg, 128.0 μmol, 25.1% yield) as a colorless oil.

¹H NMR (400 MHz, CDCl₃) δ=4.31-4.04 (m, 1H), 3.87-3.75 (m, 1H), 3.75-3.30 (m, 1H), 3.32-3.18 (m, 1H), 3.18-3.01 (m, 2H), 3.00-2.78 (m, 2H), 2.73-2.53 (m, 2H), 2.52-2.38 (m, 1H), 1.44 (s, 9H), 1.13 (t, J=6.4 Hz, 3H)

### Step C. Procedure for Preparation of (S)-tert-butyl 4-(3-(3-bromo-2-methylphenoxy)propyl)-3-(trifluoromethyl)piperazine-1-carboxylate

A mixture of (S)-tert-butyl 4-(3-hydroxypropyl)-3-(trifluoromethyl)piperazine-1-carboxylate (40 mg, 128.07 μmol, 1.00 equiv.), 3-bromo-2-methylphenol (35.93 mg, 192.11 μmol, 1.50 equiv.), and 2-(tributylphosphoranylidene)acetonitrile (46.37 mg, 192.11 μmol, 1.50 equiv.) in toluene (1 mL) was degassed and purged with N₂ three times, and then the mixture was stirred at 120° C. for 1 hour under N₂ atmosphere. The reaction mixture was diluted with water (20 mL) and extracted with EtOAc (25 mL×3). The combined organic layers were washed with brine (15 mL×3), dried over anhydrous sodium sulfate, filtered, and concentrated under reduced pressure to give (S)-tert-butyl 4-(3-(3-bromo-2-methylphenoxy)propyl)-3-(trifluoromethyl)piperazine-1-carboxylate (40 mg, 83.0 μmol, 64.9% yield) as a colorless oil.

¹H NMR (400 MHz, DMSO-d₆) δ=7.17-7.13 (m, 1H), 7.11-7.04 (m, 2H), 4.64-4.55 (m, 1H), 4.07-3.93 (m, 2H), 3.78-3.45 (m, 3H), 3.06-2.87 (m, 4H), 2.84-2.71 (m, 2H), 2.24 (d, J=3.2 Hz, 1H), 2.22 (d, J=5.2 Hz, 3H), 1.37 (d, J=2.8 Hz, 9H)

### Step D. Procedure for Preparation of (S)-1-(3-(3-bromo-2-methylphenoxy)propyl)-2-(trifluoromethyl)piperazine

A mixture of (S)-tert-butyl 4-(3-(3-bromo-2-methylphenoxy)propyl)-3-(trifluoromethyl)piperazine-1-carboxylate (40 mg, 83.10 μmol, 1 equiv.) in HCl/EtOAc (5 mL) was degassed and purged with N₂ three times, and then the mixture was stirred at 25° C. for 16 hours under N₂ atmosphere. The reaction mixture was concentrated under reduced pressure to give (S)-1-(3-(3-bromo-2-methylphenoxy)propyl)-2-(trifluoromethyl)piperazine (40 mg, crude) as a yellow oil.

¹H NMR (400 MHz, DMSO-d₆) δ=9.69 (d, J=2.4 Hz, 1H), 8.72-8.54 (m, 1H), 7.20-7.13 (m, 1H), 7.13-7.06 (m, 1H), 7.06-6.99 (m, 1H), 4.65-4.50 (m, 1H), 3.95-3.82 (m, 1H), 3.56 (s, 2H), 3.15-2.90 (m, 6H), 2.25 (d, J=7.2 Hz, 1H), 2.21 (d, J=3.6 Hz, 2H), 1.23-1.17 (m, 3H)

### Step E. Procedure for Preparation of (S)-ethyl 2-(4-(3-(3-bromo-2-methylphenoxy)propyl)-3-(trifluoromethyl)piperazin-1-yl)acetate

A mixture of ethyl 2-bromoacetate (15.99 mg, 95.76 μmol, 10.59 μL, 1.00 equiv.), (S)-1-(3-(3-bromo-2-methylphenoxy)propyl)-2-(trifluoromethyl)piperazine (40.00 mg, 95.76 μmol, 1.00 equiv., HCl), K₂CO₃ (66.18 mg, 478.82 μmol, 5.00 equiv.), KI (1.59 mg, 9.58 μmol, 0.10 equiv.) in CH₃CN (2 mL) was degassed and purged with N₂ three times, and then the mixture was stirred at 60° C. for 1 hour under N₂ atmosphere. The reaction mixture was diluted with water (20 mL) and extracted with EtOAc (25 mL×3). The combined organic layers were washed with brine (15 mL×3), dried over anhydrous sodium sulfate, filtered and concentrated under reduced pressure to give a residue. The crude product was purified by reverse-phase HPLC to give (S)-ethyl 2-(4-(3-(3-bromo-2-methylphenoxy)propyl)-3-(trifluoromethyl)piperazin-1-yl)acetate (30 mg, 64.2 μmol, 67.0% yield) as a colorless oil.

¹H NMR (400 MHz, DMSO-d₆) δ=7.14 (d, J=7.2 Hz, 1H), 7.11-7.03 (m, 2H), 4.65-4.51 (m, 1H), 4.08-4.04 (m, 2H), 3.54-3.41 (m, 1H), 3.01-2.93 (m, 2H), 2.87-2.78 (m, 2H), 2.24 (d, J=4.0 Hz, 2H), 2.21 (d, J=3.2 Hz, 3H), 1.24-1.21 (m, 4H), 1.20-1.13 (m, 6H)

### Step F. Procedure for Preparation of (S)-tert-butyl 6-(8-(benzo[d]thiazol-2-ylcarbamoyl)-3,4-dihydroisoquinolin-2(1H)-yl)-3-(3-(3-(4-(2-ethoxy-2-oxoethyl)-2-(trifluoromethyl)piperazin-1-yl)propoxy)-2-methylphenyl)picolinate

(S)-ethyl 2-(4-(3-(3-bromo-2-methylphenoxy)propyl)-3-(trifluoromethyl)piperazin-1-yl)acetate (30 mg, 64.20 μmol, 1.00 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (39.32 mg, 64.20 μmol, 1.00 equiv.), Ad₂nBuP Pd G₃ (4.68 mg, 6.42 μmol, 0.10 equiv.), and KF (1.5 M, 128.39 μL, 3.00 equiv.) were taken up into a microwave tube in dioxane (1.5 mL). The sealed tube was heated at 100° C. for 1 hour under microwave. The reaction mixture was concentrated under reduced pressure to give a residue. The residue was purified by prep-TLC (SiO₂, petroleum ether: ethyl acetate=1:1) to give a compound (S)-tert-butyl 6-(8-(benzo[d]thiazol-2-ylcarbamoyl)-3,4-dihydroisoquinolin-2(1H)-yl)-3-(3-(3-(4-(2-ethoxy-2-oxoethyl)-2-(trifluoromethyl)piperazin-1-yl)propoxy)-2-methylphenyl)picolinate (45 mg, 51.5 μmol, 80.3% yield) as a brown oil.

MS (ESI) m/z: 437.6 [M12+H]⁺

### Step G. Procedure for Preparation of (S)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-3-(trifluoromethyl)piperazin-1-yl)acetic acid

A mixture of (S)-tert-butyl 6-(8-(benzo[d]thiazol-2-ylcarbamoyl)-3,4-dihydroisoquinolin-2(1H)-yl)-3-(3-(3-(4-(2-ethoxy-2-oxoethyl)-2-(trifluoromethyl)piperazin-1-yl)propoxy)-2-methylphenyl)picolinate (45 mg, 51.55 μmol, 1.00 equiv.) and LiOH·H₂O (6.49 mg, 154.64 μmol, 3.00 equiv.) in THE (1.5 mL) and H₂O (0.5 mL) was degassed and purged with N₂ three times, and then the mixture was stirred at 40° C. for 3 hours under N₂ atmosphere. HCl (1 M) was slowly added dropwise to adjust pH to 3-4 and a white solid precipitated. The white solid was compound (S)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-3-(trifluoromethyl)piperazin-1-yl)acetic acid (50 mg, crude).

¹H NMR (400 MHz, DMSO-d₆) δ=12.99-12.68 (m, 1H), 8.05-7.99 (m, 1H), 7.81-7.75 (m, 1H), 7.60 (d, J=7.6 Hz, 1H), 7.50-7.42 (m, 3H), 7.42-7.27 (m, 3H), 7.15-7.05 (m, 1H), 7.04-6.90 (m, 2H), 6.63-6.53 (m, 1H), 5.03-4.95 (m, 2H), 4.61 (s, 1H), 3.87 (t, J=5.6 Hz, 3H), 3.20-3.10 (m, 2H), 3.09-2.99 (m, 4H), 2.99-2.88 (m, 2H), 1.92-1.87 (m, 1H), 1.85 (s, 2H), 1.36 (s, 2H), 1.25-1.15 (m, 4H), 1.05-0.97 (m, 9H)

### Step H. Procedure for Preparation of tert-butyl 6-(8-(benzo[d]thiazol-2-ylcarbamoyl)-3,4-dihydroisoquinolin-2(1H)-yl)-3-(3-(3-((2S)-4-(2-((3-(2,6-dioxopiperidin-3-yl)-1-methyl-11H-indazol-6-yl)amino)-2-oxoethyl)-2-(trifluoromethyl)piperazin-1-yl)propoxy)-2-methylphenyl)picolinate

A mixture of (S)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-3-(trifluoromethyl)piperazin-1-yl)acetic acid (50 mg, 59.18 μmol, 1.00 equiv.), 3-(6-amino-1-methyl-1H-indazol-3-yl)piperidine-2,6-dione (16.81 mg, 65.09 μmol, 1.10 equiv.), and DIEA (22.94 mg, 177.53 μmol, 30.92 μL, 3.00 equiv.) in DMF (1 mL) was degassed and purged with N₂ three times. The mixture was stirred at 25° C. for 5 minutes. After 5 minutes, HATU (27.00 mg, 71.01 μmol, 1.20 equiv.) was added, and the mixture was stirred at 25° C. for 1 hour under N₂ atmosphere. The reaction mixture was slowly added dropwise into water (10 mL), and a pink solid precipitated. The pink solid was tert-butyl 6-(8-(benzo[d]thiazol-2-ylcarbamoyl)-3,4-dihydroisoquinolin-2(1H)-yl)-3-(3-(3-((2S)-4-(2-((3-(2,6-dioxopiperidin-3-yl)-1-methyl-1H-indazol-6-yl)amino)-2-oxoethyl)-2-(trifluoromethyl)piperazin-1-yl)propoxy)-2-methylphenyl)picolinate (60 mg, crude)

MS (ESI) m/z: 543.5 [M12+H]⁺

### Step I. Procedure for preparation of 6-(8-(benzo[d]thiazol-2-ylcarbamoyl)-3,4-dihydroisoquinolin-2(1H)-yl)-3-(3-(3-((2S)-4-(2-((3-(2,6-dioxopiperidin-3-yl)-1-methyl-11H-indazol-6-yl)amino)-2-oxoethyl)-2-(trifluoromethyl)piperazin-1-yl)propoxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(3-(3-((2S)-4-(2-((3-(2,6-dioxopiperidin-3-yl)-1-methyl-1H-indazol-6-yl)amino)-2-oxoethyl)-2-(trifluoromethyl)piperazin-1-yl)propoxy)-2-methylphenyl)picolinate (60 mg, 55.29 μmol, 1.00 equiv.) in DCM (1 mL) and TFA (0.3 mL) was degassed and purged with N₂ three times, and then the mixture was stirred at 40° C. for 6 hours under N₂ atmosphere. The reaction mixture was concentrated under reduced pressure to give a residue. The residue was purified by prep-HPLC to give a compound 6-(8-(benzo[d]thiazol-2-ylcarbamoyl)-3,4-dihydroisoquinolin-2(1H)-yl)-3-(3-(3-((2S)-4-(2-((3-(2,6-dioxopiperidin-3-yl)-1-methyl-1H-indazol-6-yl)amino)-2-oxoethyl)-2-(trifluoromethyl)piperazin-1-yl)propoxy)-2-methylphenyl)picolinic acid (30.5 mg, 29.3 μmol, 53.1% yield, 99.1% purity) as a yellow solid.

MS (ESI) m/z: 1029.3 [M+H]⁺

¹H NMR (400 MHz, DMSO-d₆) δ=10.96-10.75 (m, 1H), 8.31 (s, 1H), 8.07 (s, 1H), 7.98 (d, J=8.0 Hz, 1H), 7.78-7.72 (m, 1H), 7.64 (d, J=8.0 Hz, 2H), 7.47-7.38 (m, 2H), 7.37-7.29 (m, 3H), 7.08-7.00 (m, 2H), 6.92-6.77 (m, 2H), 6.71 (d, J=7.2 Hz, 1H), 4.97 (s, 2H), 4.62-4.48 (m, 1H), 4.35-4.29 (m, 1H), 3.92-3.87 (m, 5H), 3.24 (s, 2H), 3.11 (d, J=4.4 Hz, 2H), 3.00 (s, 2H), 2.90-2.83 (m, 2H), 2.67-2.61 (m, 2H), 2.41-2.27 (m, 4H), 2.22-2.10 (m, 2H), 1.96-1.89 (m, 3H), 1.26-1.18 (m, 4H)

¹⁹F NMR (377 MHz, DMSO-d₆) δ=−63.93 (s, 1F), −73.42 (s, 1F)

### Example 64. Preparation of Compound 246b

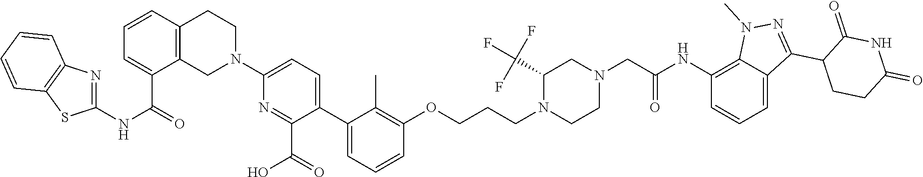 [SMILES: Cc1c(OCCCN2CCN(CC(=O)Nc3cccc4c(C5CCC(=O)NC5=O)nn(C)c34)C[C@H]2C(F)(F)F)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-((2S)-4-(2-((3-(2,6-dioxopiperidin-3-yl)-1-methyl-1H-indazol-7-yl)amino)-2-oxoethyl)-2-(trifluoromethyl)piperazin-1-yl)propoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(3-((2S)-4-(2-((3-(2,6-dioxopiperidin-3-yl)-1-methyl-11H-indazol-7-yl)amino)-2-oxoethyl)-2-(trifluoromethyl)piperazin-1-yl)propoxy)-2-methylphenyl)picolinate

A mixture of 3-(7-amino-1-methyl-1H-indazol-3-yl)piperidine-2,6-dione (37.60 mg, 145.57 μmol, 1.5 eq), (S)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-3-(trifluoromethyl)piperazin-1-yl)acetic acid (100 mg, 97.05 μmol, 82% purity, 1 equiv.), and DIEA (50.17 mg, 388.19 μmol, 67.62 μL, 4 equiv.) in DMF (1 mL) was degassed and purged with N₂ three times. The mixture was stirred at 25° C. for 5 minutes. After 5 minutes, HATU (50 mg, 131.50 μmol, 1.35 equiv.) was added, and the mixture was stirred at 25° C. for 1 hour under N₂ atmosphere. Water was added dropwise, and a yellow solid precipitated. The yellow solid was purified by prep-TLC (SiO₂, DCM:MeOH=12:1) to give tert-butyl 6-(8-(benzo[d]thiazol-2-ylcarbamoyl)-3,4-dihydroisoquinolin-2(1H)-yl)-3-(3-(3-((2S)-4-(2-((3-(2,6-dioxopiperidin-3-yl)-1-methyl-1H-indazol-7-yl)amino)-2-oxoethyl)-2-(trifluoromethyl)piperazin-1-yl)propoxy)-2-methylphenyl)picolinate (70 mg, 64.5 μmol, 66.4% yield) as a white solid.

MS (ESI) m/z: 543.5 [M12+H]⁺

### Step B. Procedure for Preparation of 6-(8-(benzo[d]thiazol-2-ylcarbamoyl)-3,4-dihydroisoquinolin-2(1H)-yl)-3-(3-(3-((2S)-4-(2-((3-(2,6-dioxopiperidin-3-yl)-1-methyl-11H-indazol-7-yl)amino)-2-oxoethyl)-2-(trifluoromethyl)piperazin-1-yl)propoxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(3-(3-((2S)-4-(2-((3-(2,6-dioxopiperidin-3-yl)-1-methyl-1H-indazol-7-yl)amino)-2-oxoethyl)-2-(trifluoromethyl)piperazin-1-yl)propoxy)-2-methylphenyl)picolinate (70 mg, 64.50 μmol, 1 equiv.) in TFA (1 mL) and DCM (1 mL) was degassed and purged with N₂ three times, and then the mixture was stirred at 40° C. for 4 hours under N₂ atmosphere. The reaction mixture was concentrated under reduced pressure to give a residue. The residue was purified by prep-HPLC to give a compound 6-(8-(benzo[d]thiazol-2-ylcarbamoyl)-3,4-dihydroisoquinolin-2(1H)-yl)-3-(3-(3-((2S)-4-(2-((3-(2,6-dioxopiperidin-3-yl)-1-methyl-1H-indazol-7-yl)amino)-2-oxoethyl)-2-(trifluoromethyl)piperazin-1-yl)propoxy)-2-methylphenyl)picolinic acid (34.5 mg, 33.4 μmol, 51.8% yield, 99.4% purity) as a yellow solid.

MS (ESI) m/z: 1029.4 [M+H]⁺

¹H NMR (400 MHz, DMSO-d₆) δ=12.85 (s, 2H), 10.90 (s, 1H), 9.81-9.57 (m, 1H), 8.13 (s, 1H), 8.03 (d, J=8.0 Hz, 1H), 7.79 (d, J=8.0 Hz, 1H), 7.65-7.56 (m, 2H), 7.49-7.43 (m, 3H), 7.40-7.32 (m, 2H), 7.23-7.18 (m, 1H), 7.12-7.05 (m, 2H), 6.99-6.91 (m, 2H), 6.66-6.61 (m, 1H), 4.98 (s, 2H), 4.65-4.50 (m, 1H), 4.42-4.34 (m, 1H), 4.07-4.02 (m, 3H), 3.92 (t, J=5.6 Hz, 2H), 3.83 (s, 2H), 3.21-3.09 (m, 2H), 3.03 (t, J=5.4 Hz, 2H), 2.95-2.83 (m, 2H), 2.74-2.61 (m, 4H), 2.43-2.28 (m, 2H), 2.27-2.25 (m, 1H), 2.26-2.00 (m, 1H), 1.93-1.86 (m, 3H), 1.24 (d, J=3.2 Hz, 4H)

¹⁹F NMR (376 MHz, DMSO-d₆) δ=−64.19-−65.16 (m, 1F), −73.55 (s, 1F)

### Example 65. Preparation of Compound 248

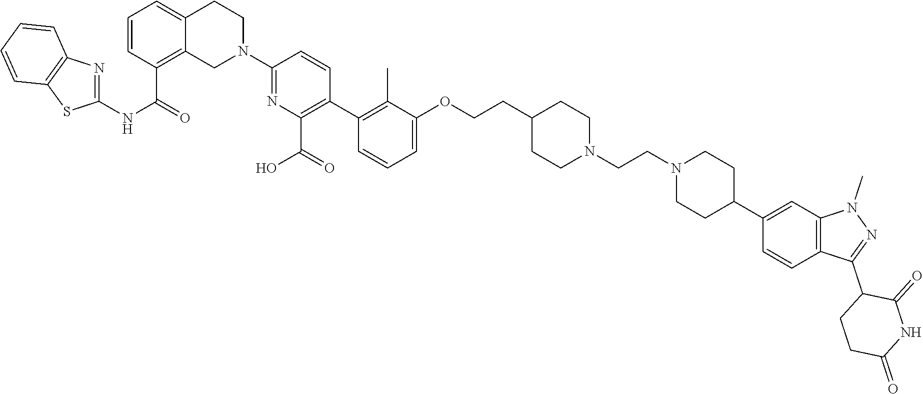 [SMILES: Cc1c(OCCC2CCN(CCN3CCC(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[2-[1-[2-[4-[3-(2,6-dioxo-3-piperidyl)-1-methyl-indazol-6-yl]-1-piperidyl]ethyl]-4-piperidyl]ethoxy]-2-methyl-phenyl]pyridine-2-carboxylic acid

### Step A. Procedure for Preparation of 6-[8-(1,3-benzothiazol-2-ylcarbamoyl)-3,4-dihydro-1H-isoquinolin-2-yl]-3-[2-methyl-3-[2-[1-(2-oxoethyl)-4-piperidyl]ethoxy]phenyl]pyridine-2-carboxylic acid

A mixture of tert-butyl 6-(8-(benzo[d]thiazol-2-ylcarbamoyl)-3,4-dihydroisoquinolin-2(1H)-yl)-3-(3-(2-(1-(2,2-diethoxyethyl)piperidin-4-yl)ethoxy)-2-methylphenyl)picolinate (200 mg, 243.89 μmol, 1.0 equiv.) in HCOOH (1.5 mL) was degassed and purged with N₂ three times, and then the mixture was stirred at 85° C. for 2 hours under N₂ atmosphere. The reaction mixture was swept with nitrogen until it was dried to give 6-[8-(1,3-benzothiazol-2-ylcarbamoyl)-3,4-dihydro-1H-isoquinolin-2-yl]-3-[2-methyl-3-[2-[1-(2-oxoethyl)-4-piperidyl]ethoxy]phenyl]pyridine-2-carboxylic acid (168.2 mg, crude) as a white oil was used into the next step without further purification.

MS (ESI) m/z: 708.1 [M+H₂O+H]⁺

### Step B. Procedure for Preparation of 6-[8-(1,3-benzothiazol-2-ylcarbamoyl)-3,4-dihydro-1H-isoquinolin-2-yl]-3-[3-[2-[1-[2-[4-[3-(2,6-dioxo-3-piperidyl)-1-methyl-indazol-6-yl]-1-piperidyl]ethyl]-4-piperidyl]ethoxy]-2-methyl-phenyl]pyridine-2-carboxylic acid

To a solution of 3-[1-methyl-6-(4-piperidyl)indazol-3-yl]piperidine-2,6-dione (79.58 mg, 243.83 μmol, 1 equiv.), NaBH(OAc)₃ (155.03 mg, 731.49 μmol, 3.0 equiv.) in DCM (5 mL) and IPA (5 mL) was added 6-[8-(1,3-benzothiazol-2-ylcarbamoyl)-3,4-dihydro-1H-isoquinolin-2-yl]-3-[2-methyl-3-[2-[1-(2-oxoethyl)-4-piperidyl]ethoxy]phenyl]pyridine-2-carboxylic acid (168.2 mg, 243.83 μmol, 1.0 equiv.) in DCM (5 mL) at 0° C. The mixture was stirred at 25° C. for 2 hours. The reaction mixture was quenched by addition H₂O (20 mL) and extracted with DCM (20 mL×3). The combined organic layers were washed with saturated brine (20 mL×2), dried over anhydrous Na₂SO₄, filtered, and concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[2-[1-[2-[4-[3-(2,6-dioxo-3-piperidyl)-1-methyl-indazol-6-yl]-1-piperidyl]ethyl]-4-piperidyl]ethoxy]-2-methyl-phenyl]pyridine-2-carboxylic acid (19.2 mg, 18.9 μmol, 7.7% yield, 98.7% purity) as a white solid.

MS (ESI) m/z: 1000.7 [M+H]⁺

¹H NMR (400 MHz, DMSO-d₆) δ=10.87 (s, 1H), 8.20 (s, 1H), 8.02 (d, J=7.6 Hz, 1H), 7.78 (d, J=8.4 Hz, 1H), 7.64-7.57 (m, 2H), 7.48-7.31 (m, 6H), 7.11-6.99 (m, 2H), 6.91-6.85 (m, 2H), 6.66 (d, J=7.6 Hz, 1H), 4.96 (s, 2H), 4.32 (dd, J=5.2, 9.6 Hz, 1H), 3.98 (t, J=4.8 Hz, 2H), 3.95 (s, 3H), 3.90 (t, J=5.6 Hz, 2H), 3.06-2.98 (m, 6H), 2.96-2.93 (m, 2H), 2.67-2.60 (m, 3H), 2.20-2.02 (m, 6H), 1.90 (s, 3H), 1.81-1.73 (m, 4H), 1.72-1.63 (m, 4H), 1.58-1.44 (m, 2H), 1.27-1.19 (m, 2H)

### Example 66. Preparation of Compound 250a

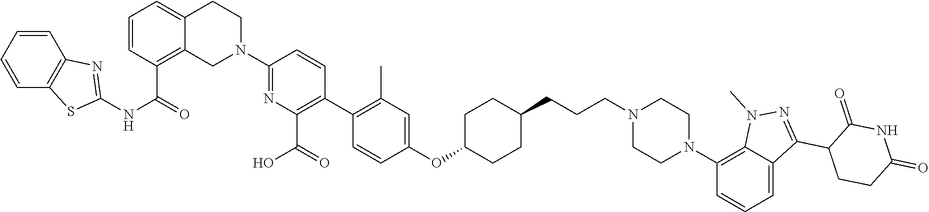 [SMILES: Cc1cc(O[C@H]2CC[C@H](CCCN3CCN(c4cccc5c(C6CCC(=O)NC6=O)nn(C)c45)CC3)CC2)ccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(4-(((1s,4r)-4-(3-(4-(3-(2,6-dioxopiperidin-3-yl)-1-methyl-1H-indazol-7-yl)piperazin-1-yl)propyl)cyclohexyl)oxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(4-(((1s,4r)-4-(3-(4-(3-(2,6-dioxopiperidin-3-yl)-1-methyl-1H-indazol-7-yl)piperazin-1-yl)propyl)cyclohexyl)oxy)-2-methylphenyl)picolinate

To a solution of 3-(1-methyl-7-piperazin-1-yl-indazol-3-yl)piperidine-2,6-dione (56.34 mg, 154.85 μmol, 1.2 equiv.) in DCM (2 mL) was added NaBH(OAc)₃ (82.05 mg, 387.12 μmol, 3 equiv.). Then tert-butyl 6-(8-(benzo[d]thiazol-2-ylcarbamoyl)-3,4-dihydroisoquinolin-2(1H)-yl)-3-(2-methyl-4-(((1r,4r)-4-(3-oxopropyl)cyclohexyl)oxy)phenyl)picolinate (94.32 mg, 129.04 μmol, 1 equiv.) was added into the mixture at 0° C. The mixture was stirred at 25° C. for 1 hour. DCM (20 mL) and water (20 mL) were added and layers were separated. The aqueous phase was extracted with DCM (10 mL×2). The combined organic extracts were dried over anhydrous sodium sulfate, filtered, and concentrated under vacuum to give tert-butyl 6-(8-(benzo[d]thiazol-2-ylcarbamoyl)-3,4-dihydroisoquinolin-2(1H)-yl)-3-(4-(((1s,4r)-4-(3-(4-(3-(2,6-dioxopiperidin-3-yl)-1-methyl-1H-indazol-7-yl)piperazin-1-yl)propyl)cyclohexyl)oxy)-2-methylphenyl)picolinate (120 mg, 115.1 μmol, 89.2% yield) as a yellow solid.

MS (ESI) m/z: 522.0 [M12+H]⁺

### Step B. Procedure for Preparation of 6-(8-(benzo[d]thiazol-2-ylcarbamoyl)-3,4-dihydroisoquinolin-2(1H)-yl)-3-(4-(((1s,4r)-4-(3-(4-(3-(2,6-dioxopiperidin-3-yl)-1-methyl-1H-indazol-7-yl)piperazin-1-yl)propyl)cyclohexyl)oxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(4-(((1s,4r)-4-(3-(4-(3-(2,6-dioxopiperidin-3-yl)-1-methyl-1H-indazol-7-yl)piperazin-1-yl)propyl)cyclohexyl)oxy)-2-methylphenyl)picolinate (120.00 mg, 115.13 μmol, 1 equiv.) in DCM (1 mL) was added TFA (1.54 g, 13.51 mmol, 1 mL, 117.31 equiv.). The mixture was stirred at 25° C. for 12 hours. The reaction mixture was concentrated under reduced pressure to remove solvent. The residue was purified by prep-HPLC to give 6-(8-(benzo[d]thiazol-2-ylcarbamoyl)-3,4-dihydroisoquinolin-2(1H)-yl)-3-(4-(((1s,4r)-4-(3-(4-(3-(2,6-dioxopiperidin-3-yl)-1-methyl-1H-indazol-7-yl)piperazin-1-yl)propyl)cyclohexyl)oxy)-2-methylphenyl)picolinic acid (41.7 mg, 41.9 μmol, 36.5% yield, 99.2% purity) as a yellow solid.

MS (ESI) m/z: 986.3 [M+H]⁺

¹H NMR (400 MHz) δ=12.88 (s, 1H), 12.65-12.52 (m, 1H), 10.90 (s, 1H), 9.57-9.44 (m, 1H), 8.04 (d, J=8.0 Hz, 1H), 7.80 (d, J=8.0 Hz, 1H), 7.63 (d, J=7.2 Hz, 1H), 7.49 (d, J=7.6 Hz, 1H), 7.47-7.43 (m, 2H), 7.40-7.33 (m, 2H), 7.13-7.05 (m, 2H), 6.98-6.89 (m, 2H), 6.79 (d, J=2.4 Hz, 1H), 6.74-6.69 (m, 1H), 4.98 (s, 2H), 4.39-4.34 (m, 1H), 4.27 (s, 3H), 4.22 (s, 1H), 3.92 (t, J=5.6 Hz, 2H), 3.62 (d, J=11.2 Hz, 2H), 3.44-3.40 (m, 2H), 3.23-3.17 (m, 2H), 3.13-3.06 (m, 2H), 3.03 (t, J=5.6 Hz, 2H), 2.71-2.62 (m, 2H), 2.55 (s, 2H), 2.36-2.31 (m, 1H), 2.21-2.14 (m, 1H), 2.13-2.07 (m, 2H), 2.03 (s, 3H), 1.86-1.80 (m, 2H), 1.72 (d, J=6.4 Hz, 2H), 1.38-1.23 (m, 5H), 1.16-1.05 (m, 2H)

### Example 67. Preparation of Compound 252a

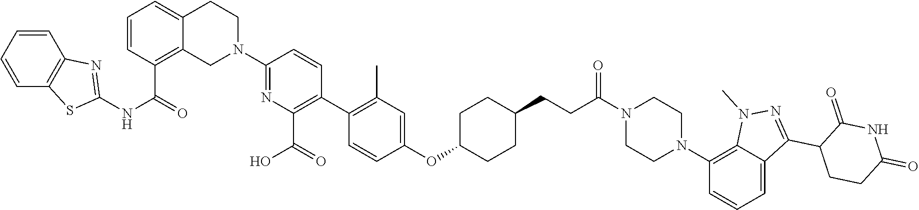 [SMILES: Cc1cc(O[C@H]2CC[C@H](CCC(=O)N3CCN(c4cccc5c(C6CCC(=O)NC6=O)nn(C)c45)CC3)CC2)ccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(4-(((1r,4r)-4-(3-(4-(3-(2,6-dioxopiperidin-3-yl)-1-methyl-1H-indazol-7-yl)piperazin-1-yl)-3-oxopropyl)cyclohexyl)oxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(4-(((1r,4r)-4-(3-(4-(3-(2,6-dioxopiperidin-3-yl)-1-methyl-1H-indazol-7-yl)piperazin-1-yl)-3-oxopropyl)cyclohexyl)oxy)-2-methylphenyl)picolinate

To a solution of 3-[4-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]cyclohexyl]propanoic acid (100 mg, 133.88 μmol, 1 equiv.) in pyridine (1 mL) was added EDCI (33.37 mg, 174.05 μmol, 1.3 equiv.) and 3-(1-methyl-7-piperazin-1-yl-indazol-3-yl)piperidine-2,6-dione (58.46 mg, 160.66 μmol, 1.2 equiv., HCl). The mixture was stirred at 40° C. for 4 hours. The reaction mixture was concentrated under reduced pressure to remove solvent. The residue was purified by column chromatography (SiO₂, petroleum ether/ethyl acetate=4/1 to 0/1, DCM:MeOH=10:1) to give tert-butyl 6-(8-(benzo[d]thiazol-2-ylcarbamoyl)-3,4-dihydroisoquinolin-2(1H)-yl)-3-(4-(((1r,4r)-4-(3-(4-(3-(2,6-dioxopiperidin-3-yl)-1-methyl-1H-indazol-7-yl)piperazin-1-yl)-3-oxopropyl)cyclohexyl)oxy)-2-methylphenyl)picolinate (140 mg, 132.5 μmol, 99.0% yield) as a yellow solid.

MS (ESI) m/z: 1056.3 [M+H]⁺

### Step B. Procedure for Preparation of 6-(8-(benzo[d]thiazol-2-ylcarbamoyl)-3,4-dihydroisoquinolin-2(1H)-yl)-3-(4-(((1r,4r)-4-(3-(4-(3-(2,6-dioxopiperidin-3-yl)-1-methyl-1H-indazol-7-yl)piperazin-1-yl)-3-oxopropyl)cyclohexyl)oxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(4-(((1r,4r)-4-(3-(4-(3-(2,6-dioxopiperidin-3-yl)-1-methyl-1H-indazol-7-yl)piperazin-1-yl)-3-oxopropyl)cyclohexyl)oxy)-2-methylphenyl)picolinate (140 mg, 132.54 μmol, 1 equiv.) in DCM (1 mL) was added TFA (1.54 g, 13.51 mmol, 1 mL, 101.90 equiv.). The mixture was stirred at 25° C. for 12 hours. The reaction mixture was concentrated under reduced pressure to remove solvent. The residue was purified by prep-HPLC to give 6-(8-(benzo[d]thiazol-2-ylcarbamoyl)-3,4-dihydroisoquinolin-2(1H)-yl)-3-(4-(((1r,4r)-4-(3-(4-(3-(2,6-dioxopiperidin-3-yl)-1-methyl-1H-indazol-7-yl)piperazin-1-yl)-3-oxopropyl)cyclohexyl)oxy)-2-methylphenyl)picolinic acid (26.3 mg, 23.9 μmol, 18.0% yield, 90.8% purity) as a yellow solid.

MS (ESI) m/z: 1000.3 [M+H]⁺

¹H NMR (400 MHz) δ=12.86 (s, 1H), 12.69-12.47 (m, 1H), 10.89 (s, 1H), 8.03 (d, J=7.6 Hz, 1H), 7.79 (d, J=8.0 Hz, 1H), 7.62 (d, J=7.2 Hz, 1H), 7.49-7.42 (m, 4H), 7.39-7.33 (m, 2H), 7.06-7.00 (m, 2H), 6.96-6.88 (m, 2H), 6.78 (d, J=2.0 Hz, 1H), 6.73-6.68 (m, 1H), 4.97 (s, 2H), 4.37-4.32 (m, 1H), 4.27 (s, 3H), 3.90 (t, J=5.6 Hz, 2H), 3.59-3.53 (m, 2H), 3.23-3.13 (m, 2H), 3.02 (t, J=5.6 Hz, 2H), 2.68-2.61 (m, 5H), 2.43-2.37 (m, 2H), 2.35-2.31 (m, 1H), 2.24-2.13 (m, 2H), 2.09-2.04 (m, 2H), 2.02 (s, 3H), 1.86-1.77 (m, 2H), 1.47 (d, J=6.4 Hz, 2H), 1.36-1.25 (m, 4H), 1.15-1.05 (m, 2H)

### Example 68. Preparation of Compound 253a

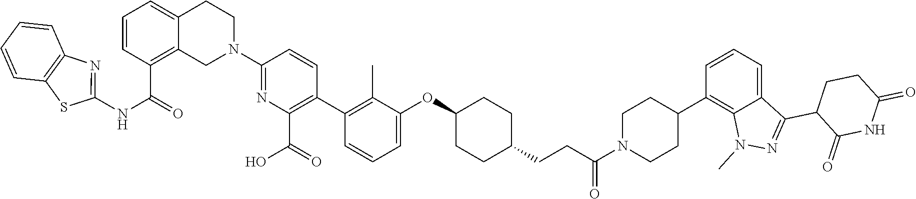 [SMILES: Cc1c(O[C@H]2CC[C@H](CCC(=O)N3CCC(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-7-yl)piperidin-1-yl)-3-oxopropyl)cyclohexyl)oxy)-2-methylphenyl)picolinic acid

### Step A. Procedure for Preparation of 3-((1r,4r)-4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)cyclohexyl)propanoic acid

To a solution of tert-butyl 6-(8-(benzo[d]thiazol-2-ylcarbamoyl)-3,4-dihydroisoquinolin-2(1H)-yl)-3-(3-(((1r,4r)-4-(3-ethoxy-3-oxopropyl)cyclohexyl)oxy)-2-methylphenyl)picolinate (75 mg, 96.78 μmol, 1 equiv.) in THF (0.75 mL) and H₂O (0.25 mL) was added LiOH·H₂O (20.31 mg, 483.89 μmol, 5 equiv.). The mixture was stirred at 25° C. for 10 hours. The reaction mixture was extracted with EtOAc (5 mL). The combined organic layers were concentrated under reduced pressure to give the crude product 3-((1r,4r)-4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)cyclohexyl)propanoic acid (60 mg, crude) as a white solid, which was used into the next step without further purification.

MS (ESI) m/z: 747.4 [M+H]⁺.

### Step B. Procedure for Preparation of tert-butyl 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-7-yl)piperidin-1-yl)-3-oxopropyl)cyclohexyl)oxy)-2-methylphenyl)picolinate

To a solution of 3-((1r,4r)-4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)cyclohexyl)propanoic acid (55 mg, 73.64 μmol, 1 equiv.) in pyridine (1 mL) was added 3-[1-methyl-7-(4-piperidyl)indazol-3-yl]piperidine-2,6-dione (36.05 mg, 110.45 μmol, 1.5 equiv.) and EDCI (42.35 mg, 220.91 μmol, 3 equiv.). The mixture was stirred at 25° C. for 2 hours. The reaction mixture was quenched by addition 1M HCl 10 (mL) at 25° C., and then diluted with H₂O 10 (mL) and extracted with DCM (10 mL). The combined organic layers were concentrated under reduced pressure to give the crude product tert-butyl 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-7-yl)piperidin-1-yl)-3-oxopropyl)cyclohexyl)oxy)-2-methylphenyl)picolinate (70 mg, crude) as a black solid, which was used into the next step without further purification.

MS (ESI) m/z: 1055.7 [M+H]⁺

### Step C. Procedure for Preparation of 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-7-yl)piperidin-1-yl)-3-oxopropyl)cyclohexyl)oxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-7-yl)piperidin-1-yl)-3-oxopropyl)cyclohexyl)oxy)-2-methylphenyl)picolinate (60 mg, 56.86 μmol, 1 equiv.) in TFA (0.5 mL) and DCM (1 mL) was degassed and purged with N₂ three times, and then the mixture was stirred at 25° C. for 16 hours under N₂ atmosphere. The reaction mixture was concentrated under reduced pressure to give a residue. The residue was purified by prep-HPLC. The compound 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-7-yl)piperidin-1-yl)-3-oxopropyl)cyclohexyl)oxy)-2-methylphenyl)picolinic acid (18.8 mg, 18.74 μmol, 32.9% yield, 99.5% purity) was obtained as a white solid.

MS (ESI) m/z: 999.6 [M+H]⁺.

¹H NMR (400 MHz, DMSO-d₆) δ=12.86 (d, J=1.2 Hz, 1H), 12.70-12.42 (m, 1H), 10.88 (s, 1H), 8.06-8.01 (m, 1H), 7.79 (d, J=8.4 Hz, 1H), 7.62 (d, J=6.4 Hz, 1H), 7.55 (d, J=8.0 Hz, 1H), 7.49-7.43 (m, 3H), 7.39-7.32 (m, 2H), 7.23 (d, J=7.2 Hz, 1H), 7.10-7.04 (m, 2H), 6.94 (dd, J=8.4, 15.6 Hz, 2H), 6.61 (d, J=7.2 Hz, 1H), 4.98 (s, 2H), 4.67-4.53 (m, 1H), 4.35 (dd, J=5.2, 9.6 Hz, 1H), 4.24 (s, 3H), 4.22-4.16 (m, 1H), 4.08-3.99 (m, 1H), 3.92 (t, J=5.7 Hz, 2H), 3.67-3.57 (m, 1H), 3.02 (t, J=5.6 Hz, 2H), 2.74-2.61 (m, 3H), 2.41-2.36 (m, 3H), 2.33 (td, J=1.8, 3.6 Hz, 1H), 2.16 (dd, J=5.6, 13.6 Hz, 1H), 2.11-2.06 (m, 2H), 1.98-1.90 (m, 2H), 1.87 (s, 3H), 1.82 (d, J=12.4 Hz, 2H), 1.72-1.65 (m, 1H), 1.60-1.52 (m, 1H), 1.50-1.44 (m, 2H), 1.39-1.28 (m, 3H), 1.15-1.06 (m, 2H)

### Example 69. Preparation of Compound 259a

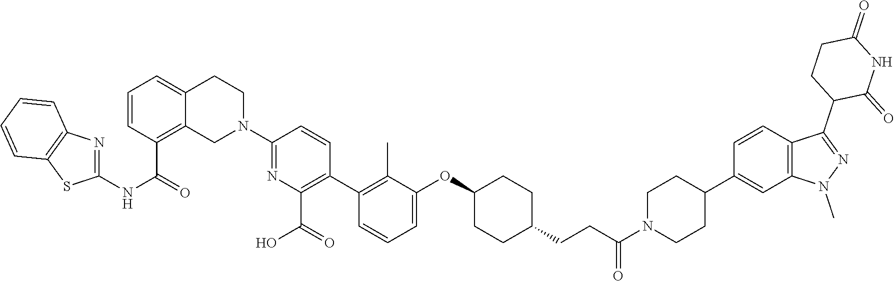 [SMILES: Cc1c(O[C@H]2CC[C@H](CCC(=O)N3CCC(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-6-yl)piperidin-1-yl)-3-oxopropyl)cyclohexyl)oxy)-2-methylphenyl)picolinic acid

### Step A. Procedure for Preparation of tert-butyl 6-[8-(1,3-benzothiazol-2-ylcarbamoyl)-3,4-dihydro-1H-isoquinolin-2-yl]-3-[3-[4-(3-ethoxy-3-oxo-propyl)cyclohexoxy]-2-methyl-phenyl]pyridine-2-carboxylate

A mixture of ethyl 3-[4-(3-bromo-2-methyl-phenoxy)cyclohexyl]propanoate (500 mg, 1.35 mmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (912.29 mg, 1.49 mmol, 1.1 equiv.), KF (1.5 M, 1.35 mL, 1.5 equiv.), and Ad₂nBuP Pd G₃(cataCXium® A Pd G₃) (295.81 mg, 406.18 μmol, 0.3 equiv.) in dioxane (15 mL) was degassed and purged with N₂ three times, and then the mixture was stirred at 100° C. for 1 hour under microwave. The reaction mixture was filtered and concentrated under reduced pressure to give a residue. The residue was purified by flash silica gel chromatography (Eluent of 0˜38% ethyl acetate/petroleum ether) to give tert-butyl 6-[8-(1,3-benzothiazol-2-ylcarbamoyl)-3,4-dihydro-1H-isoquinolin-2-yl]-3-[3-[4-(3-ethoxy-3-oxo-propyl)cyclohexoxy]-2-methyl-phenyl]pyridine-2-carboxylate (700 mg, 496.8 μmol, 36.6% yield, 55% purity) as a yellow solid.

MS (ESI) m/z: 775.5 [M+H]⁺

### Step B. Procedure for Preparation of 3-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]cyclohexyl]propanoic acid

To a solution of tert-butyl 6-[8-(1,3-benzothiazol-2-ylcarbamoyl)-3,4-dihydro-1H-isoquinolin-2-yl]-3-[3-[4-(3-ethoxy-3-oxo-propyl)cyclohexoxy]-2-methyl-phenyl]pyridine-2-carboxylate (470 mg, 606.48 μmol, 1 equiv.) in THE (5 mL) was added LiOH·H₂O (152.70 mg, 3.64 mmol, 6 equiv.) and H₂O (1 mL). The mixture was stirred at 40° C. for 1 hour. The reaction mixture was filtered and concentrated under reduced pressure to give a residue. The residue was purified by flash silica gel chromatography (Eluent of 0˜ 78% ethyl acetate/petroleum ether) to give 3-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]cyclohexyl]propanoic acid (400 mg, 535.5 μmol, 88.3% yield) was obtained as a white solid.

MS (ESI) m/z: 747.4 [M+H]⁺

### Step C. Procedure for Preparation of tert-butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-6-yl]-1-piperidyl]-3-oxo-propyl]cyclohexoxy]-2-methyl-phenyl]pyridine-2-carboxylate

A mixture of 3-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]cyclohexyl]propanoic acid (299.60 mg, 401.12 μmol, 1 equiv.), EDCI (115.34 mg, 601.68 μmol, 1.5 equiv.) in pyridine (3 mL) was degassed and purged with N₂ three times, and then the mixture was stirred at 0° C. for 1 hour under N₂ atmosphere. 3-[1-methyl-6-(4-piperidyl)indazol-3-yl]piperidine-2,6-dione (196.39 mg, 601.68 μmol, 1.5 equiv.) was added, and the mixture was stirred at 60° C. for 5 hours. The reaction mixture was filtered and concentrated under reduced pressure to give a residue. The residue was purified by flash silica gel chromatography (Eluent of 0˜ 67% ethyl acetate/petroleum ether). The compound tert-butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-6-yl]-1-piperidyl]-3-oxo-propyl]cyclohexoxy]-2-methyl-phenyl]pyridine-2-carboxylate (150 mg, 142.1 μmol, 35.4% yield) was obtained as a white solid.

MS (ESI) m/z: 528.7 [/₂M+H]⁺.

### Step D. Procedure for Preparation of 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-6-yl)piperidin-1-yl)-3-oxopropyl)cyclohexyl)oxy)-2-methylphenyl)picolinic acid

A solution of tert-butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-6-yl]-1-piperidyl]-3-oxo-propyl]cyclohexoxy]-2-methyl-phenyl]pyridine-2-carboxylate (150 mg, 142.14 μmol, 1 equiv.) in TFA (1 mL) and DCM (3 mL) was stirred at 40° C. for 1 hour. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-6-yl)piperidin-1-yl)-3-oxopropyl)cyclohexyl)oxy)-2-methylphenyl)picolinic acid (28.6 mg, 25.2 μmol, 17.7% yield, 88.0% purity) as an off-white solid.

MS (ESI) m/z: 999.0 [M+H]⁺

¹H NMR (400 MHz, DMSO-d₆) δ=10.89-10.84 (m, 1H), 8.02 (d, J=6.8 Hz, 1H), 7.78 (d, J=7.6 Hz, 1H), 7.64-7.59 (m, 2H), 7.46-7.42 (m, 3H), 7.40-7.31 (m, 2H), 7.13-6.99 (m, 3H), 6.92 (t, J=7.6 Hz, 2H), 6.62 (d, J=7.6 Hz, 1H), 4.97 (s, 2H), 4.60-4.56 (m, 1H), 4.34-4.30 (m, 1H), 4.22-4.17 (m, 1H), 4.05-4.03 (m, 1H), 3.96 (s, 3H), 3.92-3.89 (m, 2H), 3.18-3.08 (m, 2H), 3.03-3.00 (m, 2H), 2.95-2.84 (m, 1H), 2.68-2.58 (m, 4H), 2.40-2.32 (m, 4H), 2.11-2.05 (m, 2H), 1.87 (s, 3H), 1.85-1.81 (m, 2H), 1.63-1.51 (m, 2H), 1.48-1.45 (m, 2H), 1.38-1.30 (m, 3H), 1.12-1.06 (m, 2H).

### Example 70. Preparation of Compound 263a

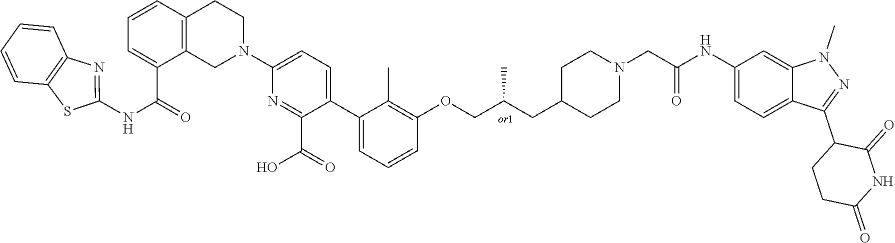 [SMILES: Cc1c(OC[C@H](C)CC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[(2R)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylic acid

### [rel-6-(8-(benzo[d]thiazol-2-ylcarbamoyl)-3,4-dihydroisoquinolin-2(1H)-yl)-3-(3-((2R)-3-(1-(2-((3-(2,6-dioxopiperidin-3-yl)-1-methyl-1H-indazol-6-yl)amino)-2-oxoethyl)piperidin-4-yl)-2-methylpropoxy)-2-methylphenyl)picolinic acid]

### Step A. Procedure for Preparation of tert-butyl 4-[(E)-3-ethoxy-2-methyl-3-oxo-prop-1-enyl]piperidine-1-carboxylate

A mixture of ethyl 2-diethoxyphosphorylpropanoate (8.38 g, 35.17 mmol, 7.69 mL, 1.50 equiv.) in THE (50 mL) was degassed and purged with N₂ three times, and then to the mixture was added LiHMDS (1 M, 46.89 mL, 2.00 equiv.) dropwise at 0° C. under N₂ atmosphere. After stirring at 0° C. for 1 hour, tert-butyl 4-formylpiperidine-1-carboxylate (5 g, 23.44 mmol, 1.00 equiv.) in THF (20 mL) was added dropwise at 0° C. The mixture was stirred at 25° C. for 15 hours. After completion, the solution was cooled to 0° C., and then added into a saturated NH₄Cl solution (100 mL) slowly. The solution was extracted with ethyl acetate (100 mL×3). The combined organic layers were washed with brine (200 mL), dried over anhydrous sodium sulfate, filtered, and concentrated under reduced pressure to give a residue. The residue was purified by flash silica gel chromatography (Eluent of 0˜25% ethyl acetate/petroleum ether) to give tert-butyl 4-[(E)-3-ethoxy-2-methyl-3-oxo-prop-1-enyl]piperidine-1-carboxylate (4.3 g, 14.4 mmol, 61.6% yield) as a colorless oil.

¹H NMR (400 MHz, DMSO-d₆) δ=6.44-6.36 (m, 1H), 5.78-5.66 (m, 1H), 4.14-3.97 (m, 2H), 3.92-3.78 (m, 2H), 2.65 (s, 2H), 1.79-1.71 (m, 3H), 1.54-1.45 (m, 2H), 1.36-1.25 (m, 10H), 1.19-1.12 (m, 3H), 1.08-1.02 (m, 1H)

### Step B. Procedure for Preparation of tert-butyl 4-(3-ethoxy-2-methyl-3-oxo-propyl)piperidine-1-carboxylate

A mixture of tert-butyl 4-[(E)-3-ethoxy-2-methyl-3-oxo-prop-1-enyl]piperidine-1-carboxylate (4.3 g, 14.46 mmol, 1.00 equiv.), Pd/C (2 g, 1.45 mmol, 10% purity, 0.10 equiv.), Pd(OH)₂ (2.03 g, 1.45 mmol, 10% purity, 0.10 equiv.) in EtOH (40 mL) was degassed and purged with H₂ (15 Psi) three times, and then the mixture was stirred at 25° C. for 16 hours under H₂ (15 Psi) atmosphere. The reaction mixture was filtered and concentrated under reduced pressure to give crude tert-butyl 4-(3-ethoxy-2-methyl-3-oxo-propyl)piperidine-1-carboxylate (3.5 g, 11.6 mmol, 80.8% yield) as a black oil.

### Step C. Procedure for Preparation of tert-butyl 4-(3-hydroxy-2-methyl-propyl)piperidine-1-carboxylate

A mixture of tert-butyl 4-(3-ethoxy-2-methyl-3-oxo-propyl)piperidine-1-carboxylate (3.5 g, 11.69 mmol, 1.00 equiv.) in THE (50 mL) was degassed and purged with N₂ for three times, and then LiAlH₄ (665.52 mg, 17.53 mmol, 1.50 equiv.) was added in portions and stirred at 25° C. for 1 hour under N₂ atmosphere. To the reaction mixture was added H₂O (0.7 mL) at 0° C., then filtered and concentrated to give a crude product. The residue was purified by flash silica gel chromatography (Eluent of 0˜30% ethyl acetate/petroleum ether) to give tert-butyl 4-(3-hydroxy-2-methyl-propyl)piperidine-1-carboxylate (2.6 g, 10.1 mmol, 86.4% yield) as a colorless oil

¹H NMR (400 MHz, DMSO-d₆) δ=4.38 (t, J=5.2 Hz, 1H), 3.97-3.85 (m, 2H), 3.29-3.21 (m, 1H), 3.20-3.12 (m, 1H), 2.78-2.56 (m, 2H), 1.65-1.54 (m, 3H), 1.51-1.43 (m, 1H), 1.39 (s, 9H), 1.28-1.21 (m, 1H), 1.00-0.85 (m, 3H), 0.82 (d, J=6.8 Hz, 3H)

### Step D. Procedure for Preparation of tert-butyl 4-[3-(3-bromo-2-methyl-phenoxy)-2-methyl-propyl]piperidine-1-carboxylate

A mixture of tert-butyl 4-(3-hydroxy-2-methyl-propyl)piperidine-1-carboxylate (2.6 g, 10.10 mmol, 1.00 equiv.), 3-bromo-2-methyl-phenol (1.89 g, 10.10 mmol, 1.00 equiv.), 2-(tributyl-λ⁵-phosphanylidene)acetonitrile (2.93 g, 12.12 mmol, 1.20 equiv.) in toluene (20 mL) was degassed and purged with N₂ three times, and then the mixture was stirred at 110° C. for 16 hours under N₂ atmosphere. The reaction mixture was concentrated under reduced pressure to give a residue. The residue was purified by flash silica gel chromatography (Eluent of 0-5% ethyl acetate/petroleum ether) to give tert-butyl 4-[3-(3-bromo-2-methyl-phenoxy)-2-methyl-propyl]piperidine-1-carboxylate (3.2 g, 7.1 mmol, 70.5% yield, 95% purity) as a colorless oil.

MS (ESI) m/z: 448.2 [M+23]⁺.

### Step E. Procedure for Preparation of 4-[3-(3-bromo-2-methyl-phenoxy)-2-methyl-propyl]piperidine

To a solution of tert-butyl 4-[3-(3-bromo-2-methyl-phenoxy)-2-methyl-propyl]piperidine-1-carboxylate (3 g, 7.04 mmol, 1.00 equiv.) in HCl/EtOAc (1 mL) was stirred at 25° C. for 0.5 hour. The reaction mixture was filtered to give crude 4-[3-(3-bromo-2-methyl-phenoxy)-2-methyl-propyl]piperidine (2 g, 5.5 mmol, 78.3% yield, HCl) as a white solid.

### Step F. Procedure for Preparation of ethyl 2-[4-[3-(3-bromo-2-methyl-phenoxy)-2-methyl-propyl]-1-piperidyl]acetate

To a solution of 4-[3-(3-bromo-2-methyl-phenoxy)-2-methyl-propyl]piperidine (2 g, 5.51 mmol, 1 equiv., HCl) in CH₃CN (20 mL) was added K₂CO₃ (2.29 g, 16.54 mmol, 3 equiv.) and ethyl 2-bromoacetate (920.80 mg, 5.51 mmol, 609.80 μL, 1 equiv.). The mixture was stirred at 40° C. for 1 hour. The reaction mixture was filtered and concentrated under reduced pressure to give a residue. The residue was purified by flash silica gel chromatography (Eluent of 0˜30% ethyl acetate/petroleum ether) to give ethyl 2-[4-[3-(3-bromo-2-methyl-phenoxy)-2-methyl-propyl]-1-piperidyl]acetate (2.2 g, 5.3 mmol, 96.7% yield) as a colorless oil.

MS (ESI) m/z: 412.2 [M+H]⁺.

### Step G. Procedure for Preparation of ethyl 2-[4-[(2R)-3-(3-bromo-2-methyl-phenoxy)-2-methyl-propyl]-1-piperidyl]acetate

Ethyl 2-[4-[3-(3-bromo-2-methyl-phenoxy)-2-methyl-propyl]-1-piperidyl]acetate (1 g, 2.43 mmol, 1.00 equiv.) was separated by SFC to give ethyl 2-[4-[(2R)-3-(3-bromo-2-methyl-phenoxy)-2-methyl-propyl]-1-piperidyl]acetate (0.29 g, 703.2 μmol, 29.0% yield) as a colorless oil.

Step H. Procedure for Preparation of tert-butyl 6-[8-(1,3-benzothiazol-2-ylcarbamoyl)-3,4-dihydro-1H-isoquinolin-2-yl]-3-[3-[(2R)-3-[1-(2-ethoxy-2-oxo-ethyl)-4-piperidyl]-2-methyl-propoxy]-2-methyl-phenyl]pyridine-2-carboxylate

A mixture of ethyl 2-[4-[(2R)-3-(3-bromo-2-methyl-phenoxy)-2-methyl-propyl]-1-piperidyl]acetate (150 mg, 363.76 μmol, 1.00 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (222.82 mg, 363.76 μmol, 1.00 equiv.), Ad₂nBuP Pd G₃ (264.92 mg, 363.76 μmol, 1.00 equiv.), and K₃PO₄ (231.64 mg, 1.09 mmol, 3.00 equiv.) in dioxane (2 mL) and H₂O (0.2 mL) was degassed and purged with N₂ three times, and then the mixture was stirred at 75° C. for 16 hours under N₂ atmosphere. The reaction mixture was concentrated under reduced pressure to give a residue. The residue was purified by prep-TLC (SiO₂, petroleum ether/ethyl acetate=1:1) to give tert-butyl 6-[8-(1,3-benzothiazol-2-ylcarbamoyl)-3,4-dihydro-1H-isoquinolin-2-yl]-3-[3-[(2R)-3-[1-(2-ethoxy-2-oxo-ethyl)-4-piperidyl]-2-methyl-propoxy]-2-methyl-phenyl]pyridine-2-carboxylate (150 mg, 183.3 μmol, 50.4% yield) was obtained as a yellow solid.

MS (ESI) m/z: 818.5 [M+H]⁺.

### Step I. Procedure for preparation of 2-[4-[(2R)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-2-methyl-propyl]-1-piperidyl]acetic acid

To a solution of tert-butyl 6-[8-(1,3-benzothiazol-2-ylcarbamoyl)-3,4-dihydro-1H-isoquinolin-2-yl]-3-[3-[(2R)-3-[1-(2-ethoxy-2-oxo-ethyl)-4-piperidyl]-2-methyl-propoxy]-2-methyl-phenyl]pyridine-2-carboxylate (140 mg, 171.14 μmol, 1.00 equiv.) in THE (2 mL) was added LiOH·H₂O (21.55 mg, 513.43 μmol, 3.00 equiv.) and H₂O (0.5 mL). The mixture was stirred at 25° C. for 3 minutes. The reaction mixture was concentrated under reduced pressure to give crude 2-[4-[(2R)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-2-methyl-propyl]-1-piperidyl]acetic acid (100 mg, 126.5 μmol, 73.9% yield) as a yellow solid.

MS (ESI) m/z: 790.5 [M+H]⁺.

### Step J. Procedure for Preparation of tert-butyl 6-[8-(1,3-benzothiazol-2-ylcarbamoyl)-3,4-dihydro-1H-isoquinolin-2-yl]-3-[3-[(2R)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylate

To a solution of 2-[4-[(2R)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-2-methyl-propyl]-1-piperidyl]acetic acid (80 mg, 101.27 μmol, 1.00 equiv.) and 3-(6-amino-1-methyl-indazol-3-yl)piperidine-2,6-dione (28.77 mg, 111.40 μmol, 1.10 equiv.) in DMF (1 mL) was added HATU (46.21 mg, 121.52 μmol, 1.20 equiv.) and TEA (30.74 mg, 303.81 μmol, 42.29 μL, 3.00 equiv.). The mixture was stirred at 40° C. for 1 hour. The solution was poured into water (2 mL) and filtered to give tert-butyl 6-[8-(1,3-benzothiazol-2-ylcarbamoyl)-3,4-dihydro-1H-isoquinolin-2-yl]-3-[3-[(2R)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylate (80 mg, 77.6 μmol, 76.6% yield) as a pink solid.

MS (ESI) m/z: 1030.5 [M+H]⁺.

### Step K. Procedure for Preparation of 6-[8-(1,3-benzothiazol-2-ylcarbamoyl)-3,4-dihydro-1H-isoquinolin-2-yl]-3-[3-[(2R)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylic acid [rel-6-(8-(benzo[d]thiazol-2-ylcarbamoyl)-3,4-dihydroisoquinolin-2(1H)-yl)-3-(3-((2R)-3-(1-(2-((3-(2,6-dioxopiperidin-3-yl)-1-methyl-1H-indazol-6-yl)amino)-2-oxoethyl)piperidin-4-yl)-2-methylpropoxy)-2-methylphenyl)picolinic acid]

To a solution of tert-butyl 6-[8-(1,3-benzothiazol-2-ylcarbamoyl)-3,4-dihydro-1H-isoquinolin-2-yl]-3-[3-[(2R)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylate (80 mg, 77.65 μmol, 1.00 equiv.) in DCM (1 mL) was added TFA (616.00 mg, 5.40 mmol, 400.00 μL, 69.57 equiv.). The mixture was stirred at 40° C. for 16 hours. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[(2R)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylic acid [rel-6-(8-(benzo[d]thiazol-2-ylcarbamoyl)-3,4-dihydroisoquinolin-2(1H)-yl)-3-(3-((2R)-3-(1-(2-((3-(2,6-dioxopiperidin-3-yl)-1-methyl-1H-indazol-6-yl)amino)-2-oxoethyl)piperidin-4-yl)-2-methylpropoxy)-2-methylphenyl)picolinic acid] (28.5 mg, 27.7 μmol, 35.7% yield, 99.1% purity) as a yellow solid.

MS (ESI) m/z: 488.0 [M12+H]⁺.

¹H NMR (400 MHz, DMSO-d₆) δ=13.36-11.83 (m, 1H), 10.88 (s, 1H), 9.83 (s, 1H), 8.16 (s, 1H), 8.08-7.97 (m, 2H), 7.78 (d, J=8.0 Hz, 1H), 7.62 (d, J=8.4 Hz, 2H), 7.49-7.41 (m, 3H), 7.40-7.31 (m, 2H), 7.21 (dd, J=1.6, 8.8 Hz, 1H), 7.12-7.05 (m, 1H), 6.95 (d, J=8.8 Hz, 1H), 6.87 (d, J=8.4 Hz, 1H), 6.63 (d, J=7.6 Hz, 1H), 4.98 (s, 2H), 4.32 (dd, J=5.2, 9.6 Hz, 1H), 3.95-3.86 (m, 5H), 3.83-3.73 (m, 2H), 3.13 (s, 2H), 3.02 (t, J=5.2 Hz, 2H), 2.91-2.83 (m, 2H), 2.67-2.59 (m, 2H), 2.20-2.14 (m, 2H), 2.02 (s, 1H), 1.95-1.87 (m, 3H), 1.74-1.61 (m, 2H), 1.49-1.37 (m, 2H), 1.36-1.09 (m, 4H), 1.04-0.97 (m, 3H)

### Example 71. Preparation of Compound 267

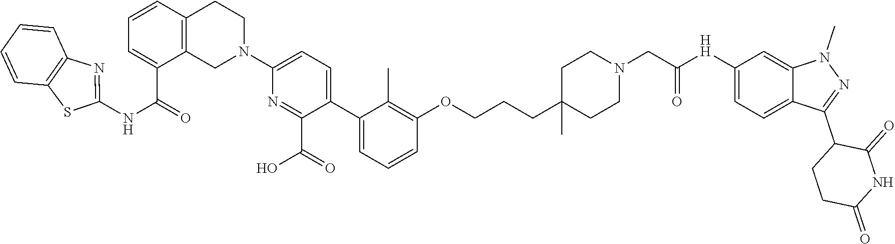

### 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-2-oxo-ethyl]-4-methyl-4-piperidyl]propoxy]-2-methyl-phenyl]pyridine-2-carboxylic acid

### Step A. Procedure for Preparation of tert-butyl 4-[(E)-3-ethoxy-3-oxo-prop-1-enyl]-4-methyl-piperidine-1-carboxylate

To a solution of ethyl 2-diethoxyphosphorylacetate (1.97 g, 8.80 mmol, 1.75 mL, 2.0 equiv.) in THE (10 mL) was added LiHMDS (1 M, 13.20 mL, 3.0 equiv.) at 0° C., and then the reaction mixture was stirred at 0° C. for 1 hour. After that, tert-butyl 4-formyl-4-methyl-piperidine-1-carboxylate (1 g, 4.40 mmol, 1.0 equiv.) was added into above mixture, and the resulting mixture was stirred at 25° C. for 11 hours. The reaction mixture was quenched by adding saturated ammonium chloride solution (10 mL) at 25° C. It was then diluted with EtOAc (10 mL) and extracted with EtOAc 30 mL (10 mL×3). The combined organic layers were washed with brine (30 mL), dried over anhydrous sodium sulfate, filtered, and concentrated under reduced pressure to tert-butyl 4-[(E)-3-ethoxy-3-oxo-prop-1-enyl]-4-methyl-piperidine-1-carboxylate (1.08 g, crude) as a yellow oil.

¹H NMR (400 MHz, DMSO-d₆) δ=6.85 (d, J=16.0 Hz, 1H), 5.83 (d, J=16.0 Hz, 1H), 4.15-4.08 (m, 2H), 3.37 (d, J=4.0 Hz, 2H), 3.26-3.19 (m, 2H), 1.62-1.53 (m, 2H), 1.38 (s, 9H), 1.36-1.32 (m, 2H), 1.21 (t, J=7.2 Hz, 3H), 1.05 (s, 3H).

### Step B. Procedure for Preparation of tert-butyl 4-(3-ethoxy-3-oxo-propyl)-4-methyl-piperidine-1-carboxylate

A mixture of tert-butyl 4-[(E)-3-ethoxy-3-oxo-prop-1-enyl]-4-methyl-piperidine-1-carboxylate (1.08 g, 3.63 mmol, 1.0 equiv.) Pd/C (500 mg, 10% purity, 1.0 equiv.) in MeOH (10 mL) was degassed and purged with H₂ three times, and then the mixture was stirred at 25° C. for 12 hours under H₂ atmosphere. The reaction mixture was filtered and concentrated under reduced pressure to give tert-butyl 4-(3-ethoxy-3-oxo-propyl)-4-methyl-piperidine-1-carboxylate (1.06 g, 3.5 mmol, 97.4% yield) as a yellow oil.

¹H NMR (400 MHz, CDCl₃) δ=4.18-4.09 (m, 2H), 3.58 (d, J=12.8 Hz, 3H), 3.25-3.15 (m, 3H), 2.41 (s, 1H), 2.32-2.23 (m, 2H), 1.66-1.60 (m, 3H), 1.46 (s, 9H), 1.27 (t, J=7.2 Hz, 3H), 0.93 (s, 3H).

### Step C. Procedure for Preparation of tert-butyl 4-(3-hydroxypropyl)-4-methyl-piperidine-1-carboxylate

To a solution of tert-butyl 4-(3-ethoxy-3-oxo-propyl)-4-methyl-piperidine-1-carboxylate (1 g, 3.34 mmol, 1.0 equiv.) in THE (10 mL) was added LiAlH₄ (152.12 mg, 4.01 mmol, 1.2 equiv.). The mixture was stirred at 0° C. for 1 hour. The reaction mixture was quenched with saturated sodium sulfate solution (0.456 mL) at 0° C., filtered, and concentrated to give a residue. The residue was purified by column chromatography (SiO₂, petroleum ether/ethyl acetate 10/1 to 2/1) to give tert-butyl 4-(3-hydroxypropyl)-4-methyl-piperidine-1-carboxylate (500 mg, 1.9 mmol, 58.1% yield) as a colorless oil.

¹H NMR (400 MHz, DMSO-d₆) δ=4.36 (t, J=5.2 Hz, 1H), 3.45-3.39 (m, 2H), 3.36 (d, J=5.6 Hz, 2H), 3.13 (d, J=5.6 Hz, 2H), 1.38 (s, 9H), 1.36-1.31 (m, 2H), 1.26-1.20 (m, 6H), 0.88-0.86 (m, 3H).

### Step D. Procedure for Preparation of tert-butyl 4-[3-(3-bromo-2-methyl-phenoxy)propyl]-4-methyl-piperidine-1-carboxylate

To a solution of tert-butyl 4-(3-hydroxypropyl)-4-methyl-piperidine-1-carboxylate (250 mg, 971.37 μmol, 1.0 equiv.) in toluene (3 mL) and 3-bromo-2-methyl-phenol (218.01 mg, 1.17 mmol, 1.2 equiv.) was added 2-(tributyl-λ5-phosphanylidene)acetonitrile (351.67 mg, 1.46 mmol, 1.5 equiv.). The mixture was stirred at 120° C. for 12 hours. The reaction mixture was concentrated under reduced pressure to give a residue. The residue was purified by column chromatography (SiO₂, petroleum ether/ethyl acetate 1/0 to 20/1) to give tert-butyl 4-[3-(3-bromo-2-methyl-phenoxy)propyl]-4-methyl-piperidine-1-carboxylate (200 mg, 469.0 μmol, 48.2% yield) as a yellow oil.

¹H NMR (400 MHz, DMSO-d₆) δ=7.16-7.13 (m, 1H), 7.12-7.06 (m, 1H), 6.95 (d, J=8.0 Hz, 1H), 3.96 (t, J=6.4 Hz, 2H), 3.45-3.40 (m, 2H), 3.22-3.12 (m, 2H), 2.23 (s, 3H), 1.74-1.64 (m, 2H), 1.41-1.36 (m, 11H), 1.31-1.24 (m, 4H), 0.92 (s, 3H).

### Step E. Procedure for Preparation of 4-[3-(3-bromo-2-methyl-phenoxy)propyl]-4-methyl-piperidine

A solution of tert-butyl 4-[3-(3-bromo-2-methyl-phenoxy)propyl]-4-methyl-piperidine-1-carboxylate (200 mg, 469.06 μmol, 1.0 equiv.) in HCl/dioxane (2 mL) was stirred at 25° C. for 1 hour. The reaction mixture was concentrated under reduced pressure to give compound 4-[3-(3-bromo-2-methyl-phenoxy)propyl]-4-methyl-piperidine (240 mg, crude) as a white solid.

### Step F. Procedure for Preparation of ethyl 2-[4-[3-(3-bromo-2-methyl-phenoxy)propyl]-4-methyl-1-piperidyl]acetate

To a solution of 4-[3-(3-bromo-2-methyl-phenoxy)propyl]-4-methyl-piperidine (240 mg, 735.59 μmol, 1.0 equiv.) and ethyl 2-bromoacetate (98.27 mg, 588.47 μmol, 65.08 μL, 1.0 equiv.) in CH₃CN (2 mL) was added K₂CO₃ (304.99 mg, 2.21 mmol, 3.0 equiv.). The mixture was stirred at 60° C. for 12 hours. The reaction mixture was filtered and concentrated under reduced pressure to give ethyl 2-[4-[3-(3-bromo-2-methyl-phenoxy)propyl]-4-methyl-1-piperidyl]acetate (300 mg, 727.5 μmol, 98.9% yield) as a yellow oil.

MS (ESI) m/z: 412.2 [M+H]⁺

¹H NMR (400 MHz, DMSO-d₆) δ=7.17-7.13 (m, 1H), 7.12-7.06 (m, 1H), 6.95 (d, J=7.6 Hz, 1H), 4.11-4.04 (m, 2H), 3.96 (t, J=6.4 Hz, 2H), 3.18 (s, 2H), 2.53 (s, 1H), 2.48-2.47 (m, 1H), 2.46-2.39 (m, 2H), 2.24 (s, 3H), 1.75-1.60 (m, 2H), 1.40-1.29 (m, 6H), 1.20-1.16 (m, 3H), 0.88 (s, 3H).

### Step G. Procedure for Preparation of tert-butyl 6-[8-(1,3-benzothiazol-2-ylcarbamoyl)-3,4-dihydro-1H-isoquinolin-2-yl]-3-[3-[3-[1-(2-ethoxy-2-oxo-ethyl)-4-methyl-4-piperidyl]propoxy]-2-methyl-phenyl]pyridine-2-carboxylate

Ethyl 2-[4-[3-(3-bromo-2-methyl-phenoxy)propyl]-4-methyl-1-piperidyl]acetate (300 mg, 727.52 μmol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (534.77 mg, 873.02 μmol, 1.2 equiv.), [2-(2-aminophenyl)phenyl]palladium(1+); bis(1-adamantyl)-butyl-phosphane; methanesulfonate (52.98 mg, 72.75 μmol, 0.1 equiv.), and KF (1.5 M, 1.46 mL, 3 equiv.) were taken up into a microwave tube in dioxane (3 mL). The sealed tube was heated at 100° C. for 1 hour under microwave. The reaction mixture was filtered and concentrated under reduced pressure to give a residue. The residue was purified by column chromatography (SiO₂, petroleum ether/ethyl acetate 10/1 to 1/1) to give tert-butyl 6-[8-(1,3-benzothiazol-2-ylcarbamoyl)-3,4-dihydro-1H-isoquinolin-2-yl]-3-[3-[3-[1-(2-ethoxy-2-oxo-ethyl)-4-methyl-4-piperidyl]propoxy]-2-methyl-phenyl]pyridine-2-carboxylate (320 mg, 391.1 μmol, 53.7% yield) as a yellow solid.

MS (ESI) m/z: 818.5 [M+H]⁺

### Step H. Procedure for Preparation of 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-4-methyl-1-piperidyl]acetic acid

To a solution of tert-butyl 6-[8-(1,3-benzothiazol-2-ylcarbamoyl)-3,4-dihydro-1H-isoquinolin-2-yl]-3-[3-[3-[1-(2-ethoxy-2-oxo-ethyl)-4-methyl-4-piperidyl]propoxy]-2-methyl-phenyl]pyridine-2-carboxylate (120 mg, 146.69 μmol, 1.0 equiv.) in THE (1 mL) and H₂O (1 mL) was added LiOH·H₂O (13 mg, 1.47 mmol, 10 equiv.). The mixture was stirred at 25° C. for 1 hour. The reaction mixture was adjusted to pH=˜4 with HCl (1 M) and extracted with DCM (2 mL). The combined organic layers were concentrated under reduced pressure to give 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-4-methyl-1-piperidyl]acetic acid (100 mg, 126.5 μmol, 86.2% yield) as a white solid.

### Step I. Procedure for preparation of tert-butyl 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-2-oxo-ethyl]-4-methyl-4-piperidyl]propoxy]-2-methyl-phenyl]pyridine-2-carboxylate

To a solution of 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-4-methyl-1-piperidyl]acetic acid (120 mg, 151.90 μmol, 1.0 equiv.) and 3-(6-amino-1-methyl-indazol-3-yl)piperidine-2,6-dione (47.08 mg, 182.28 μmol, 1.2 equiv.) in pyridine (1 mL) was added EDCI (43.68 mg, 227.85 μmol, 1.5 equiv.). The mixture was stirred at 25° C. for 1 hour. The reaction mixture was quenched by adding water (5 mL), and then filtered and concentrated under reduced pressure to give compound tert-butyl 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-2-oxo-ethyl]-4-methyl-4-piperidyl]propoxy]-2-methyl-phenyl]pyridine-2-carboxylate (100 mg, 97.0 μmol, 63.9% yield) as a yellow solid.

MS (ESI) m/z: 1030.4 [M+H]⁺

### Step J. Procedure for Preparation of 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-2-oxo-ethyl]-4-methyl-4-piperidyl]propoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-2-oxo-ethyl]-4-methyl-4-piperidyl]propoxy]-2-methyl-phenyl]pyridine-2-carboxylate (100 mg, 97.06 μmol, 1.0 equiv.) in DCM (1 mL) and TFA (1 mL). The mixture was stirred at 25° C. for 12 hours. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-2-oxo-ethyl]-4-methyl-4-piperidyl]propoxy]-2-methyl-phenyl]pyridine-2-carboxylic acid (48.9 mg, 49.5 μmol, 51.0% yield, 98.6% purity) as a white solid.

MS (ESI) m/z: 974.4 [M+H]⁺

¹H NMR (400 MHz, DMSO-d₆) δ=12.96-12.74 (m, 1H), 10.87 (s, 1H), 8.02 (d, J=8.0 Hz, 2H), 7.79 (d, J=8.0 Hz, 1H), 7.70-7.59 (m, 2H), 7.50-7.42 (m, 3H), 7.40-7.32 (m, 2H), 7.19-7.14 (m, 1H), 7.10 (t, J=8.0 Hz, 1H), 6.97 (d, J=8.8 Hz, 1H), 6.89 (d, J=8.4 Hz, 1H), 6.63 (d, J=7.6 Hz, 1H), 4.98 (s, 2H), 4.38-4.28 (m, 1H), 3.97 (d, J=5.2 Hz, 2H), 3.95-3.88 (m, 5H), 3.23-3.13 (m, 2H), 3.03 (t, J=6.0 Hz, 2H), 2.72-2.55 (m, 5H), 2.37-2.30 (m, 1H), 2.23-2.12 (m, 1H), 1.92 (s, 3H), 1.82-1.37 (m, 9H), 0.97 (s, 3H).

### Example 72. Preparation of Compound 272a

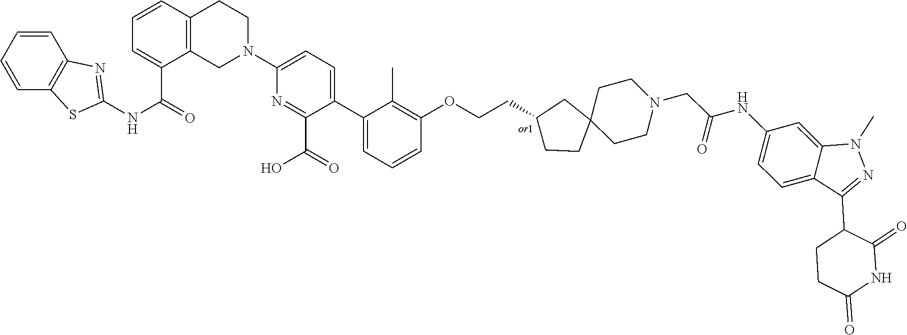 [SMILES: Cc1c(OCC[C@H]2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(2-((2S)-8-(2-((3-(2,6-dioxopiperidin-3-yl)-1-methyl-1H-indazol-6-yl)amino)-2-oxoethyl)-8-azaspiro[4.5]decan-2-yl)ethoxy)-2-methylphenyl)picolinic acid

### [rel-6-(8-(benzo[d]thiazol-2-ylcarbamoyl)-3,4-dihydroisoquinolin-2(1H)-yl)-3-(3-(2-((2S)-8-(2-((3-(2,6-dioxopiperidin-3-yl)-1-methyl-1H-indazol-6-yl)amino)-2-oxoethyl)-8-azaspiro[4.5]decan-2-yl)ethoxy)-2-methylphenyl)picolinic acid]

### Step A. Procedure for Preparation of tert-butyl (E)-2-(2-ethoxy-2-oxoethylidene)-8-azaspiro[4.5]decane-8-carboxylate

To a solution of NaH (331.54 mg, 8.29 mmol, 60% purity, 2.1 equiv.) in THE (10 mL) was added dropwise ethyl 2-(diethoxyphosphoryl)acetate (1.77 g, 7.89 mmol, 1.57 mL, 2 equiv.) in THE (5 mL) at 0° C. for 15 min under N₂ atmosphere. Then tert-butyl 2-oxo-8-azaspiro[4.5]decane-8-carboxylate (1 g, 3.95 mmol, 1 equiv.) was added at 0° C. under N₂ atmosphere and the mixture was stirred for 2 hours. The reaction mixture was quenched by addition NH₄Cl (15 mL) at 0° C., and then diluted with H₂O (10 mL) and extracted with ethyl acetate 30 mL (10 mL×3). The combined organic layers were washed with NaHCO₃ 30 mL (10 mL×3), dried over Na₂SO₄, filtered, and concentrated under reduced pressure to give a residue. The residue was purified by flash silica gel chromatography (Eluent of 0˜ 20% ethyl acetate/petroleum ether) to give tert-butyl (E)-2-(2-ethoxy-2-oxoethylidene)-8-azaspiro[4.5]decane-8-carboxylate (0.8 g, 2.4 mmol, 62.6% yield) as a white solid.

¹H NMR (400 MHz, DMSO-d₆) δ=5.76 (s, 1H), 4.05 (q, J=7.2 Hz, 2H), 3.32-3.15 (m, 5H), 2.76-2.72 (m, 1H), 2.64-2.51 (m, 1H), 2.37 (s, 1H), 1.74-1.52 (m, 2H), 1.42-1.29 (m, 13H), 1.22-1.14 (m, 3H)

### Step B. Procedure for Preparation of tert-butyl 2-(2-ethoxy-2-oxoethyl)-8-azaspiro[4.5]decane-8-carboxylate

A mixture of tert-butyl (E)-2-(2-ethoxy-2-oxoethylidene)-8-azaspiro[4.5]decane-8-carboxylate (0.8 g, 2.47 mmol, 1 equiv.), Pd/C (0.2 g, 10% purity) in EtOH (10 mL) was degassed and purged with N₂ three times, and then the mixture was stirred at 25° C. for 2 hours under H₂ (15 Psi) atmosphere. The reaction mixture was concentrated under reduced pressure to give a residue. The residue was used for next step without other purification. The compound tert-butyl 2-(2-ethoxy-2-oxoethyl)-8-azaspiro[4.5]decane-8-carboxylate (800 mg, 2.1 mmol, 88.5% yield, 89% purity) was obtained as a colorless oil.

MS (ESI) m/z: 348.2 [M+22]⁺.

### Step C. Procedure for Preparation of tert-butyl 2-(2-hydroxyethyl)-8-azaspiro[4.5]decane-8-carboxylate

To a solution of tert-butyl 2-(2-ethoxy-2-oxoethyl)-8-azaspiro[4.5]decane-8-carboxylate (800 mg, 2.46 mmol, 1 equiv.) in THE (10 mL) was added LAH (111.96 mg, 2.95 mmol, 1.2 equiv.) under 0° C. The mixture was stirred at 0° C. for 2 hours. The reaction mixture was quenched by addition Na₂SO₄·10H₂O (300 mg) under 0° C. and N₂ atmosphere, and then filtered and concentrated under reduced pressure to give a residue. The residue was used for next step without other purification. The compound tert-butyl 2-(2-hydroxyethyl)-8-azaspiro[4.5]decane-8-carboxylate (800 mg, 2.1 mmol, 86.2% yield, 75% purity) was obtained as a colorless oil.

¹H NMR (400 MHz, DMSO-d₆) δ=4.33 (d, J=3.2 Hz, 1H), 3.37 (t, J=6.8 Hz, 2H), 3.28-3.23 (m, 3H), 1.98-1.90 (m, 1H), 1.78-1.67 (m, 2H), 1.59-1.39 (m, 4H), 1.38 (s, 9H), 1.36-1.26 (m, 5H), 1.20-1.12 (m, 1H), 0.91 (dd, J=10.0, 12.8 Hz, 1H)

### Step D. Procedure for Preparation of tert-butyl 2-(2-(3-bromo-2-methylphenoxy)ethyl)-8-azaspiro[4.5]decane-8-carboxylate

A mixture of tert-butyl 2-(2-hydroxyethyl)-8-azaspiro[4.5]decane-8-carboxylate (750.06 mg, 2.65 mmol, 1.1 equiv.), 3-bromo-2-methyl-phenol (450 mg, 2.41 mmol, 1 equiv.), 2-(tributyl-λ⁵-phosphanylidene)acetonitrile (871.04 mg, 3.61 mmol, 1.5 equiv.) in toluene (8 mL) was degassed and purged with N₂ three times, and then the mixture was stirred at 120° C. for 10 hours under N₂ atmosphere. The reaction mixture was filtered and concentrated under reduced pressure to give a residue. The residue was purified by flash silica gel chromatography (Eluent of 0˜20% ethyl acetate/petroleum ether) to give tert-butyl 2-(2-(3-bromo-2-methylphenoxy)ethyl)-8-azaspiro[4.5]decane-8-carboxylate (800 mg, 1.3 mmol, 55.1% yield, 75% purity) as a colorless oil.

¹H NMR (400 MHz, DMSO-d₆) δ=7.17-7.12 (m, 1H), 7.11-7.05 (m, 1H), 6.99-6.95 (m, 1H), 3.98 (t, J=6.4 Hz, 2H), 3.26 (d, J=4.4 Hz, 4H), 2.23 (s, 3H), 2.14-2.02 (m, 1H), 1.83-1.72 (m, 4H), 1.43-1.28 (m, 16H), 1.10-0.99 (m, 1H)

### Step E. Procedure for Preparation of 2-(2-(3-bromo-2-methylphenoxy)ethyl)-8-azaspiro[4.5]decane

A solution of tert-butyl 2-(2-(3-bromo-2-methylphenoxy)ethyl)-8-azaspiro[4.5]decane-8-carboxylate (800 mg, 1.33 mmol, 75% purity, 1 equiv.) in HCl/dioxane (4 M, 8 mL) was stirred at 25° C. for 2 hours. The reaction mixture was concentrated under reduced pressure to give a residue. The residue was used for next step without other purification. The compound 2-(2-(3-bromo-2-methylphenoxy)ethyl)-8-azaspiro[4.5]decane (300 mg, 771.7 μmol, 58.1% yield, HCl) was obtained as a white solid.

MS (ESI) m/z: 352.0 [M+H]⁺.

### Step F. Procedure for Preparation of ethyl 2-[3-[2-(3-bromo-2-methyl-phenoxy)ethyl]-8-azaspiro[4.5]decan-8-yl]acetate

A mixture of 2-(2-(3-bromo-2-methylphenoxy)ethyl)-8-azaspiro[4.5]decane (300 mg, 851.53 μmol, 1 equiv.), ethyl 2-bromoacetate (142.21 mg, 851.53 μmol, 94.18 μL, 1 equiv.), K₂CO₃ (353.06 mg, 2.55 mmol, 3 equiv.), KI (70.68 mg, 425.76 μmol, 0.5 equiv.) in CH₃CN (5 mL) was degassed and purged with N₂ three times, and then the mixture was stirred at 60° C. for 2 hours under N₂ atmosphere. The reaction mixture was filtered and concentrated under reduced pressure to give a residue. The residue was purified by flash silica gel chromatography (Eluent of 0˜10% ethyl acetate/petroleum ether). The compound ethyl 2-[3-[2-(3-bromo-2-methyl-phenoxy)ethyl]-8-azaspiro[4.5]decan-8-yl]acetate (200 mg, 452.8 μmol, 53.1% yield, 99.2% purity) was obtained as a colorless oil.

¹H NMR (400 MHz, DMSO-d₆) δ=7.16-7.11 (m, 1H), 7.11-7.04 (m, 1H), 6.95 (d, J=8.0 Hz, 1H), 4.06 (q, J=7.2 Hz, 2H), 3.96 (t, J=6.4 Hz, 2H), 3.19-3.15 (m, 2H), 2.47-2.37 (m, 4H), 2.22 (s, 3H), 2.10-2.01 (m, 1H), 1.82-1.69 (m, 4H), 1.46-1.33 (m, 6H), 1.27-1.21 (m, 1H), 1.17 (t, J=7.2 Hz, 3H), 0.98 (dd, J=10.2, 12.4 Hz, 1H)

### Step G. Procedure for Preparation of ethyl (S)-2-(2-(2-(3-bromo-2-methylphenoxy)ethyl)-8-azaspiro[4.5]decan-8-yl)acetate

The compound ethyl 2-[3-[2-(3-bromo-2-methyl-phenoxy)ethyl]-8-azaspiro[4.5]decan-8-yl]acetate (200 mg, 452.88 μmol, 53.18% yield, 99.27% purity) was purified by SFC to give ethyl (R)-2-(2-(2-(3-bromo-2-methylphenoxy)ethyl)-8-azaspiro[4.5]decan-8-yl)acetate (50 mg, 114.05 μmol, 25.00% yield) as a colorless oil, and ethyl (S)-2-(2-(2-(3-bromo-2-methylphenoxy)ethyl)-8-azaspiro[4.5]decan-8-yl)acetate (66 mg, 150.5 μmol, 33.0% yield) as a colorless oil.

### Step H. Procedure for Preparation of tert-butyl (S)-6-(8-(benzo[d]thiazol-2-ylcarbamoyl)-3,4-dihydroisoquinolin-2(1H)-yl)-3-(3-(2-(8-(2-ethoxy-2-oxoethyl)-8-azaspiro[4.5]decan-2-yl)ethoxy)-2-methylphenyl)picolinate

A mixture of ethyl (S)-2-(2-(2-(3-bromo-2-methylphenoxy)ethyl)-8-azaspiro[4.5]decan-8-yl)acetate (66 mg, 150.55 μmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (129.10 mg, 210.77 μmol, 1.4 equiv.), [2-(2-aminophenyl)phenyl]palladium(1+); bis(1-adamantyl)-butyl-phosphane; methanesulfonate (21.93 mg, 30.11 μmol, 0.2 equiv.), K₂CO₃ (1.5 M, 150.55 μL, 1.5 equiv.) in dioxane (0.7 mL) was degassed and purged with N₂ three times, and then the mixture was stirred at 100° C. for 1 hour under M. W. The reaction mixture was filtered and concentrated under reduced pressure to give a residue. The residue was purified by flash silica gel chromatography (Eluent of 0˜50% ethyl acetate/petroleum ether) to give tert-butyl (S)-6-(8-(benzo[d]thiazol-2-ylcarbamoyl)-3,4-dihydroisoquinolin-2(1H)-yl)-3-(3-(2-(8-(2-ethoxy-2-oxoethyl)-8-azaspiro[4.5]decan-2-yl)ethoxy)-2-methylphenyl)picolinate (120 mg, 142.1 μmol, 94.4% yield) as a colorless oil.

MS (ESI) m/z: 844.4 [M+H]⁺.

### Step I. Procedure for Preparation of (S)-2-(2-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethyl)-8-azaspiro[4.5]decan-8-yl)acetic acid

A mixture of tert-butyl (S)-6-(8-(benzo[d]thiazol-2-ylcarbamoyl)-3,4-dihydroisoquinolin-2(1H)-yl)-3-(3-(2-(8-(2-ethoxy-2-oxoethyl)-8-azaspiro[4.5]decan-2-yl)ethoxy)-2-methylphenyl)picolinate (120 mg, 142.17 μmol, 1 equiv.), LiOH·H2O (17.90 mg, 426.51 μmol, 3 equiv.) in THE (3 mL) and H₂O (1 mL) was stirred at 25° C. for 10 hours. The mixture was concentrated, and the pH was adjusted to 3, then triturated and filtered to obtain a residue. The residue was used for next step without other purification. The compound (S)-2-(2-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethyl)-8-azaspiro[4.5]decan-8-yl)acetic acid (100 mg, 122.5 μmol, 86.2% yield) was obtained as a white solid.

MS (ESI) m/z: 816.4 [M+H]⁺.

### Step J. Procedure for Preparation of tert-butyl 6-[8-(1,3-benzothiazol-2-ylcarbamoyl)-3,4-dihydro-1H-isoquinolin-2-yl]-3-[3-[2-[(3S)-8-[2-[[3-(2,6-dioxo-3-piperidyl)-1-methyl-indazol-6-yl]amino]-2-oxo-ethyl]-8-azaspiro[4.5]decan-3-yl]ethoxy]-2-methyl-phenyl]pyridine-2-carboxylate

A mixture of (S)-2-(2-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethyl)-8-azaspiro[4.5]decan-8-yl)acetic acid (100 mg, 122.55 μmol, 1 equiv.), 3-(6-amino-1-methyl-1H-indazol-3-yl)piperidine-2,6-dione (37.98 mg, 147.06 μmol, 1.2 equiv.), EDCI (35.24 mg, 183.82 μmol, 1.5 equiv.) in pyridine (1 mL) was degassed and purged with N₂ three times, and then the mixture was stirred at 25° C. for 2 hours under N₂ atmosphere. The reaction mixture was filtered and concentrated under reduced pressure to give a residue. The residue was used for next step without other purification. The compound tert-butyl 6-[8-(1,3-benzothiazol-2-ylcarbamoyl)-3,4-dihydro-1H-isoquinolin-2-yl]-3-[3-[2-[(3S)-8-[2-[[3-(2,6-dioxo-3-piperidyl)-1-methyl-indazol-6-yl]amino]-2-oxo-ethyl]-8-azaspiro[4.5]decan-3-yl]ethoxy]-2-methyl-phenyl]pyridine-2-carboxylate (100 mg, 94.7 μmol, 77.2% yield) was obtained as a colorless oil.

MS (ESI) m/z: 1056.4 [M+H]⁺.

### Step K. Procedure for Preparation of 6-(8-(benzo[d]thiazol-2-ylcarbamoyl)-3,4-dihydroisoquinolin-2(1H)-yl)-3-(3-(2-((2S)-8-(2-((3-(2,6-dioxopiperidin-3-yl)-1-methyl-11H-indazol-6-yl)amino)-2-oxoethyl)-8-azaspiro[4.5]decan-2-yl)ethoxy)-2-methylphenyl)picolinic acid [rel-6-(8-(benzo[d]thiazol-2-ylcarbamoyl)-3,4-dihydroisoquinolin-2(1H)-yl)-3-(3-(2-((2S)-8-(2-((3-(2,6-dioxopiperidin-3-yl)-1-methyl-1H-indazol-6-yl)amino)-2-oxoethyl)-8-azaspiro[4.5]decan-2-yl)ethoxy)-2-methylphenyl)picolinic acid]

A solution of tert-butyl 6-[8-(1,3-benzothiazol-2-ylcarbamoyl)-3,4-dihydro-1H-isoquinolin-2-yl]-3-[3-[2-[(3S)-8-[2-[[3-(2,6-dioxo-3-piperidyl)-1-methyl-indazol-6-yl]amino]-2-oxo-ethyl]-8-azaspiro[4.5]decan-3-yl]ethoxy]-2-methyl-phenyl]pyridine-2-carboxylate (100 mg, 94.67 μmol, 1 equiv.) in TFA (0.5 mL) and DCM (0.5 mL) was stirred at 25° C. for 10 hours. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(2-((2R)-8-(2-((3-(2,6-dioxopiperidin-3-yl)-1-methyl-1H-indazol-6-yl)amino)-2-oxoethyl)-8-azaspiro[4.5]decan-2-yl)ethoxy)-2-methylphenyl)picolinic acid [rel-6-(8-(benzo[d]thiazol-2-ylcarbamoyl)-3,4-dihydroisoquinolin-2(1H)-yl)-3-(3-(2-((2S)-8-(2-((3-(2,6-dioxopiperidin-3-yl)-1-methyl-1H-indazol-6-yl)amino)-2-oxoethyl)-8-azaspiro[4.5]decan-2-yl)ethoxy)-2-methylphenyl)picolinic acid] (14.8 mg, 14.5 μmol, 15.3% yield, 97.9% purity) as a yellow solid.

MS (ESI) m/z: 1000.4 [M+H]⁺.

¹H NMR (400 MHz, DMSO-d₆) δ=12.98-12.71 (m, 1H), 10.87 (s, 1H), 9.95-9.69 (m, 1H), 8.06-8.00 (m, 2H), 7.78 (d, J=8.0 Hz, 1H), 7.66-7.59 (m, 2H), 7.49-7.21 (m, 5H), 7.22 (s, 1H), 7.09 (t, J=8.0 Hz, 1H), 6.96 (d, J=9.2 Hz, 1H), 6.89 (d, J=8.0 Hz, 1H), 6.63 (d, J=7.6 Hz, 1H), 4.98 (s, 2H), 4.35-4.28 (m, 1H), 4.00-3.88 (m, 8H), 3.17-2.99 (m, 4H), 2.67-2.60 (m, 4H), 2.39-2.29 (m, 2H), 2.21-2.08 (m, 2H), 1.89 (s, 3H), 1.85-1.75 (m, 4H), 1.60-1.43 (m, 6H), 1.39-1.20 (m, 2H)

### Example 73. Preparation of Compound 273b

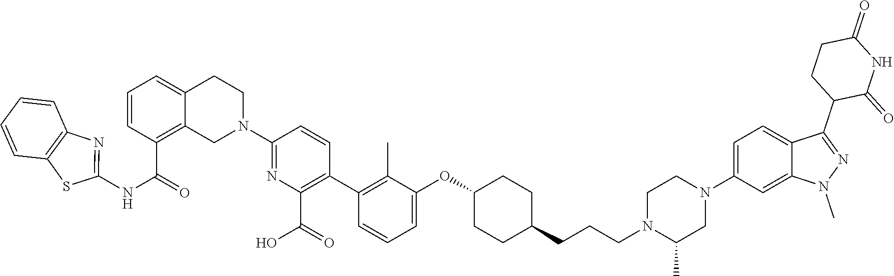 [SMILES: Cc1c(O[C@H]2CC[C@H](CCCN3CCN(c4ccc5c(C6CCC(=O)NC6=O)nn(C)c5c4)C[C@@H]3C)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1R,4r)-4-(3-((2S)-4-(3-(2,6-dioxopiperidin-3-yl)-1-methyl-1H-indazol-6-yl)-2-methylpiperazin-1-yl)propyl)cyclohexyl)oxy)-2-methylphenyl)picolinic acid

### Step A. Procedure for Preparation of tert-butyl (2S)-4-[3-(2,6-dibenzyloxy-3-pyridyl)-1-methyl-indazol-6-yl]-2-methyl-piperazine-1-carboxylate

A mixture of 6-bromo-3-(2,6-dibenzyloxy-3-pyridyl)-1-methyl-indazole (1.5 g, 3.00 mmol, 1.0 equiv.), tert-butyl (2S)-2-methylpiperazine-1-carboxylate (1.80 g, 9.00 mmol, 3.0 equiv.), RuPhos (279.77 mg, 600.00 μmol, 0.2 equiv.), Cs₂CO₃ (2.93 g, 9.00 mmol, 3.0 equiv.), and Pd₂(dba)₃ (274.50 mg, 300.00 μmol, 0.1 equiv.) in toluene (15 mL) was degassed and purged with N₂ three times, and then the mixture was stirred at 110° C. for 16 hours under N₂ atmosphere. The reaction mixture was concentrated under reduced pressure to remove toluene. The resulting residue was purified by flash silica gel chromatography (Eluent of 16% ethyl acetate/petroleum ether) to give tert-butyl (2S)-4-[3-(2,6-dibenzyloxy-3-pyridyl)-1-methyl-indazol-6-yl]-2-methyl-piperazine-1-carboxylate (1.2 g, 1.8 mmol, 61.1% yield, 94.7% purity) was obtained as brown oil.

MS (ESI) m/z: 620.6 [M+H]⁺.

### Step B. Procedure for Preparation of tert-butyl (2S)-4-[3-(2,6-dioxo-3-piperidyl)-1-methyl-indazol-6-yl]-2-methyl-piperazine-1-carboxylate

A mixture of tert-butyl (2S)-4-[3-(2,6-dibenzyloxy-3-pyridyl)-1-methyl-indazol-6-yl]-2-methyl-piperazine-1-carboxylate (1.20 g, 1.94 mmol, 1 equiv.), Pd/C (300 mg, 1.94 mmol, 10% purity, 1.00 equiv.), Pd(OH)₂ (300 mg, 2.14 mmol, 1.10 equiv.), and AcOH (116.28 mg, 1.94 mmol, 110.74 uL, 1.0 equiv.) in THE (10 mL) and EtOH (10 mL) was degassed and purged with N₂ three times, and then the mixture was stirred at 25° C. for 16 hours under N₂ (15 Psi) atmosphere. The mixture was filtered, and filter cake was washed by THF (20 mL). The filtrate was concentrated under reduced pressure to give tert-butyl (2S)-4-[3-(2,6-dioxo-3-piperidyl)-1-methyl-indazol-6-yl]-2-methyl-piperazine-1-carboxylate (900 mg, 1.4 mmol, 74.5% yield, 70.8% purity) as brown oil.

MS (ESI) m/z: 442.2 [M+H]⁺.

### Step C. Procedure for Preparation of 3-[1-methyl-6-[(3S)-3-methylpiperazin-1-yl]indazol-3-yl]piperidine-2,6-dione

A mixture of tert-butyl (2S)-4-[3-(2,6-dioxo-3-piperidyl)-1-methyl-indazol-6-yl]-2-methyl-piperazine-1-carboxylate (900.00 mg, 2.04 mmol, 1 equiv.) in a solvent of DCM (5 mL) and HCl/dioxane (10 mL) was stirred at 25° C. for 2 hours. The reaction mixture was treated with EtOAc (20 mL) and then filtered. The filtrate was concentrated under reduced pressure to give 3-[1-methyl-6-[(3S)-3-methylpiperazin-1-yl]indazol-3-yl]piperidine-2,6-dione (600 mg, 1.7 mmol, 86.2% yield) as a greyish-green solid.

MS (ESI) m/z: 342.1 [M+H]⁺.

### Step D. Procedure for Preparation of tert-butyl 6-[8-(1,3-benzothiazol-2-ylcarbamoyl)-3,4-dihydro-1H-isoquinolin-2-yl]-3-[3-[4-[3-[(2S)-4-[3-(2,6-dioxo-3-piperidyl)-1-methyl-indazol-6-yl]-2-methyl-piperazin-1-yl]propyl]cyclohexoxy]-2-methyl-phenyl]pyridine-2-carboxylate

A mixture of tert-butyl 6-[8-(1,3-benzothiazol-2-ylcarbamoyl)-3,4-dihydro-1H-isoquinolin-2-yl]-3-[2-methyl-3-[4-(3-oxopropyl)cyclohexoxy]phenyl]pyridine-2-carboxylate (150 mg, 205.22 μmol, 1.0 equiv.) and 3-[1-methyl-6-[(3S)-3-methylpiperazin-1-yl]indazol-3-yl]piperidine-2,6-dione (84.08 mg, 246.27 μmol, 1.2 equiv.), NaBH(OAc)₃ (130.49 mg, 615.67 μmol, 3.0 equiv.) in DCM (3 mL), and then the mixture was stirred at 25° C. for 3.5 hours. The reaction mixture was diluted with water (20 mL) and extracted with ethyl acetate (20 mL×3). The combined organic layers were washed with brine (10 mL×2), dried over Na₂SO₄, filtered, and concentrated under reduced pressure to give a residue. The residue was purified by prep-TLC (SiO₂, DCM:MeOH=10:1) to give compound tert-butyl 6-[8-(1,3-benzothiazol-2-ylcarbamoyl)-3,4-dihydro-1H-isoquinolin-2-yl]-3-[3-[4-[3-[(2S)-4-[3-(2,6-dioxo-3-piperidyl)-1-methyl-indazol-6-yl]-2-methyl-piperazin-1-yl]propyl]cyclohexoxy]-2-methyl-phenyl]pyridine-2-carboxylate (60 mg, 55.8 μmol, 27.2% yield, 98.3% purity) as a brown solid.

MS (ESI) m/z: 1057.6 [M+H]⁺.

### Step E. Procedure for Preparation of 6-(8-(benzo[d]thiazol-2-ylcarbamoyl)-3,4-dihydroisoquinolin-2(1H)-yl)-3-(3-(((1R,4r)-4-(3-((2S)-4-(3-(2,6-dioxopiperidin-3-yl)-1-methyl-1H-indazol-6-yl)-2-methylpiperazin-1-yl)propyl)cyclohexyl)oxy)-2-methylphenyl)picolinic acid

To a solution of tert-butyl 6-[8-(1,3-benzothiazol-2-ylcarbamoyl)-3,4-dihydro-1H-isoquinolin-2-yl]-3-[3-[4-[3-[(2S)-4-[3-(2,6-dioxo-3-piperidyl)-1-methyl-indazol-6-yl]-2-methyl-piperazin-1-yl]propyl]cyclohexoxy]-2-methyl-phenyl]pyridine-2-carboxylate (50 mg, 47.33 μmol, 1.0 equiv.) in DCM (1 mL) was added TFA (1 mL). The mixture was stirred at 25° C. for 16 hours. The reaction mixture was concentrated under reduced pressure to remove DCM and TFA to give a residue. The crude product was purified by prep-HPLC to give 6-(8-(benzo[d]thiazol-2-ylcarbamoyl)-3,4-dihydroisoquinolin-2(1H)-yl)-3-(3-(((1R,4r)-4-(3-((2S)-4-(3-(2,6-dioxopiperidin-3-yl)-1-methyl-1H-indazol-6-yl)-2-methylpiperazin-1-yl)propyl)cyclohexyl)oxy)-2-methylphenyl)picolinic acid (31.1 mg, 27.9 μmol, 58.9% yield, 89.5% purity) as a brown solid.

MS (ESI) m/z: 1000.5 [M+H]⁺

¹H NMR (400 MHz, DMSO-d₆) δ=12.95-12.79 (m, 1H), 10.90 (s, 1H), 8.03 (d, J=7.6 Hz, 1H), 7.79 (d, J=7.2 Hz, 1H), 7.62 (d, J=6.0 Hz, 1H), 7.51-7.42 (m, 4H), 7.40-7.29 (m, 2H), 7.12-7.04 (m, 3H), 6.95 (dd, J=8.4, 13.8 Hz, 2H), 6.66-6.58 (m, 1H), 4.98 (s, 2H), 4.36 (dd, J=5.2, 9.6 Hz, 1H), 4.26 (s, 4H), 3.94-3.88 (m, 2H), 3.74-3.56 (m, 2H), 3.03 (t, J=5.6 Hz, 4H), 2.93-2.84 (m, 1H), 2.68-2.61 (m, 2H), 2.54 (s, 6H), 2.37-2.29 (m, 2H), 2.19-2.05 (m, 4H), 1.91-1.79 (m, 6H), 1.38-1.26 (m, 6H), 1.15-1.06 (m, 2H).

### Example 74. Preparation of Compound 274a

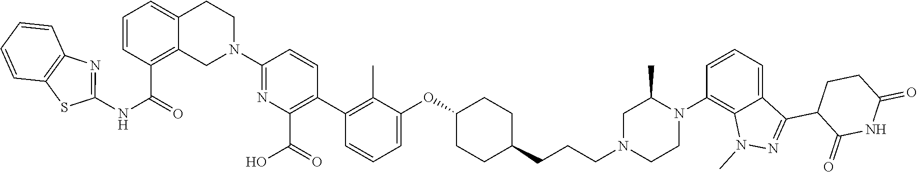 [SMILES: Cc1c(O[C@H]2CC[C@H](CCCN3CCN(c4cccc5c(C6CCC(=O)NC6=O)nn(C)c45)[C@H](C)C3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1S,4r)-4-(3-((3R)-4-(3-(2,6-dioxopiperidin-3-yl)-1-methyl-1H-indazol-7-yl)-3-methylpiperazin-1-yl)propyl)cyclohexyl)oxy)-2-methylphenyl)picolinic acid

### Step A. Procedure for Preparation of tert-butyl (3R)-4-[3-(2,6-dibenzyloxy-3-pyridyl)-1-methyl-indazol-7-yl]-3-methyl-piperazine-1-carboxylate

A mixture of 7-bromo-3-(2,6-dibenzyloxy-3-pyridyl)-1-methyl-indazole (1.5 g, 3.00 mmol, 1 equiv.), tert-butyl (3R)-3-methylpiperazine-1-carboxylate (900.55 mg, 4.50 mmol, 1.5 equiv.), Cs₂CO₃ (2.93 g, 8.99 mmol, 3 equiv.) and 1,3-bis[2,6-bis(1-propylbutyl)phenyl]-4,5-dichloro-2H-imidazol-1-ium-2-ide; 3-chloropyridine; dichloropalladium (145.80 mg, 149.88 μmol, 0.05 equiv.) in 2-methyl-2-butanol (15 mL) was degassed and purged with N₂ three times, and then the mixture was stirred at 100° C. for 16 hours under N₂ atmosphere. The reaction mixture was filtered and diluted with H₂O (30 mL) and extracted with EtOAc (30 mL×3). The combined organic layers were washed with brine (50 mL), dried over Na₂SO₄, filtered, and concentrated under reduced pressure to give a residue. The residue was purified by flash silica gel chromatography (Eluent of 0˜30% ethyl acetate/petroleum ether) to afford tert-butyl (3R)-4-[3-(2,6-dibenzyloxy-3-pyridyl)-1-methyl-indazol-7-yl]-3-methyl-piperazine-1-carboxylate (260 mg, 385.9 μmol, 12.8% yield, 92% purity) as a light yellow oil.

MS (ESI) m/z: 620.4 [M+H]⁺.

### Step B. Procedure for Preparation of tert-butyl (3R)-4-[3-(2,6-dioxo-3-piperidyl)-1-methyl-indazol-7-yl]-3-methyl-piperazine-1-carboxylate

To a solution of tert-butyl (3R)-4-[3-(2,6-dibenzyloxy-3-pyridyl)-1-methyl-indazol-7-yl]-3-methyl-piperazine-1-carboxylate (300 mg, 484.07 μmol, 1 equiv.) in THF (5 mL) and EtOH (5 mL) was added Pd/C (100 mg, 484.07 μmol, 10% purity), Pd(OH)₂ (100 mg, 71.20 μmol, 10% purity) and AcOH (87.21 mg, 1.45 mmol, 83.05 μL, 3 equiv.) under N₂ atmosphere. The suspension was degassed and purged with H₂ three times. The mixture was stirred under H₂ (15 Psi) at 25° C. for 16 hours. The reaction mixture was filtered was washed with THE (30 mL). The filtrate was concentrated to afford tert-butyl (3R)-4-[3-(2,6-dioxo-3-piperidyl)-1-methyl-indazol-7-yl]-3-methyl-piperazine-1-carboxylate (300 mg, crude) as a black brown oil, which was used in the next step without further purification.

### Step C. Procedure for Preparation of 3-[1-methyl-7-[(2R)-2-methylpiperazin-1-yl]indazol-3-yl]piperidine-2,6-dione

To a solution of tert-butyl (3R)-4-[3-(2,6-dioxo-3-piperidyl)-1-methyl-indazol-7-yl]-3-methyl-piperazine-1-carboxylate (300 mg, 679.47 μmol, 1 equiv.) in dioxane (5 mL) was added HCl/EtOAc (4 M, 169.87 μL, 1 equiv.). The mixture was stirred at 25° C. for 2 hours. The reaction mixture was filtered to afford 3-[1-methyl-7-[(2R)-2-methylpiperazin-1-yl]indazol-3-yl]piperidine-2,6-dione (150 mg, crude) as a light yellow solid, which was used in the next step without further purification.

Step D. Procedure for Preparation of tert-butyl 6-[8-(1,3-benzothiazol-2-ylcarbamoyl)-3,4-dihydro-1H-isoquinolin-2-yl]-3-[3-[4-[3-[(3R)-4-[3-(2,6-dioxo-3-piperidyl)-1-methyl-indazol-7-yl]-3-methyl-piperazin-1-yl]propyl]cyclohexoxy]-2-methyl-phenyl]pyridine-2-carboxylate

To a solution of 3-[1-methyl-7-[(2R)-2-methylpiperazin-1-yl]indazol-3-yl]piperidine-2,6-dione (77.07 mg, 225.75 μmol, 1.1 equiv.) in DCM (2 mL) was added tert-butyl 6-[8-(1,3-benzothiazol-2-ylcarbamoyl)-3,4-dihydro-1H-isoquinolin-2-yl]-3-[2-methyl-3-[4-(3-oxopropyl)cyclohexoxy]phenyl]pyridine-2-carboxylate (150 mg, 205.22 μmol, 1 equiv.). The mixture was stirred at 25° C. for 1 hour, and then treated with NaBH(OAc)₃ (130.49 mg, 615.67 μmol, 3 equiv.). The mixture was stirred at 25° C. for 16 hours. The reaction mixture was filtered and concentrated to give a residue. The residue was purified by prep-TLC (SiO2, DCM:MeOH=15:1) to afford tert-butyl 6-[8-(1,3-benzothiazol-2-ylcarbamoyl)-3,4-dihydro-1H-isoquinolin-2-yl]-3-[3-[4-[3-[(3R)-4-[3-(2,6-dioxo-3-piperidyl)-1-methyl-indazol-7-yl]-3-methyl-piperazin-1-yl]propyl]cyclohexoxy]-2-methyl-phenyl]pyridine-2-carboxylate (140 mg, 107.3 μmol, 52.3% yield, 81% purity) as a light yellow solid

MS (ESI) m/z: 529.1 [M12+H]⁺.

### Step E. Procedure for Preparation of 6-(8-(benzo[d]thiazol-2-ylcarbamoyl)-3,4-dihydroisoquinolin-2(1H)-yl)-3-(3-(((1S,4r)-4-(3-((3R)-4-(3-(2,6-dioxopiperidin-3-yl)-1-methyl-1H-indazol-7-yl)-3-methylpiperazin-1-yl)propyl)cyclohexyl)oxy)-2-methylphenyl)picolinic acid

To a solution of tert-butyl 6-[8-(1,3-benzothiazol-2-ylcarbamoyl)-3,4-dihydro-1H-isoquinolin-2-yl]-3-[3-[4-[3-[(3R)-4-[3-(2,6-dioxo-3-piperidyl)-1-methyl-indazol-7-yl]-3-methyl-piperazin-1-yl]propyl]cyclohexoxy]-2-methyl-phenyl]pyridine-2-carboxylate (140 mg, 132.54 μmol, 1 equiv.) in DCM (1 mL) was added TFA (1.54 g, 13.51 mmol, 1 mL, 101.91 equiv.). The mixture was stirred at 25° C. for 16 hours. The reaction mixture was concentrated under reduced pressure to give a residue. The residue was purified by prep-HPLC to afford 6-(8-(benzo[d]thiazol-2-ylcarbamoyl)-3,4-dihydroisoquinolin-2(1H)-yl)-3-(3-(((1S,4r)-4-(3-((3R)-4-(3-(2,6-dioxopiperidin-3-yl)-1-methyl-1H-indazol-7-yl)-3-methylpiperazin-1-yl)propyl)cyclohexyl)oxy)-2-methylphenyl)picolinic acid (42.9 mg, 38.9 μmol, 29.3% yield, 90.6% purity) as a white solid.

MS (ESI) m/z: 501.1[M/2+H]⁺

¹H NMR (400 MHz, DMSO-d₆) δ=10.88 (s, 1H), 8.03 (d, J=7.6 Hz, 1H), 7.79 (d, J=8.0 Hz, 1H), 7.62 (d, J=7.2 Hz, 1H), 7.50-7.42 (m, 4H), 7.39-7.32 (m, 2H), 7.27-7.20 (m, 1H), 7.11-7.04 (m, 2H), 6.98-6.90 (m, 2H), 6.61 (d, J=7.2 Hz, 1H), 4.98 (s, 2H), 4.36-4.32 (m, 1H), 4.27 (s, 3H), 4.23-4.16 (m, 1H), 3.94-3.81 (m, 2H), 3.05-3.00 (m, 3H), 2.90-2.84 (m, 1H), 2.82-2.73 (m, 2H), 2.68-2.61 (m, 3H), 2.39-2.28 (m, 4H), 2.21-2.15 (m, 1H), 2.11-2.05 (m, 2H), 1.95-1.90 (m, 1H), 1.87 (s, 3H), 1.84-1.77 (m, 2H), 1.53-1.46 (m, 2H), 1.41-1.33 (m, 2H), 1.29-1.20 (m, 3H), 1.12-1.02 (m, 2H), 0.87-0.77 (m, 3H)

### Example 75. Preparation of Compound 274b

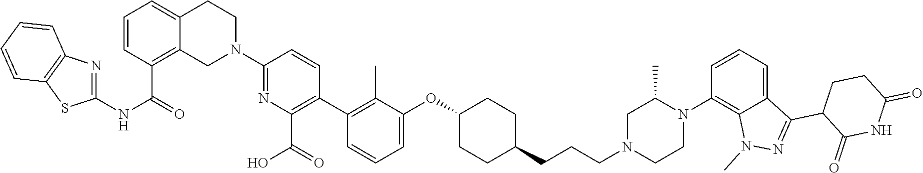 [SMILES: Cc1c(O[C@H]2CC[C@H](CCCN3CCN(c4cccc5c(C6CCC(=O)NC6=O)nn(C)c45)[C@@H](C)C3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1R,4r)-4-(3-((3S)-4-(3-(2,6-dioxopiperidin-3-yl)-1-methyl-1H-indazol-7-yl)-3-methylpiperazin-1-yl)propyl)cyclohexyl)oxy)-2-methylphenyl)picolinic acid

### Step A. Procedure for Preparation of tert-butyl (3S)-4-[3-(2,6-dibenzyloxy-3-pyridyl)-1-methyl-indazol-7-yl]-3-methyl-piperazine-1-carboxylate

A mixture of 7-bromo-3-(2,6-dibenzyloxy-3-pyridyl)-1-methyl-indazole (1.7 g, 3.40 mmol, 1.0 equiv.), tert-butyl (3S)-3-methylpiperazine-1-carboxylate (1.02 g, 5.10 mmol, 1.5 equiv.), Cs₂CO₃ (3.32 g, 10.19 mmol, 3.0 equiv.), and 1,3-bis[2,6-bis(1-propylbutyl)phenyl]-4,5-dichloro-2H-imidazol-1-ium-2-ide; 3-chloropyridine; dichloro palladium (165.24 mg, 169.87 μmol, 0.05 equiv.) in 2-methyl-2-butanol (15 mL) was degassed and purged with N₂ three times, and then the mixture was stirred at 100° C. for 16 hours under N₂ atmosphere. The reaction mixture was concentrated under reduced pressure to remove solvent. The residue was diluted with H₂O (100 mL) and extracted with ethyl acetate (100 mL×3). The combined organic layers were dried over Na₂SO₄, filtered, and concentrated under reduced pressure to give a residue. The residue was purified by prep-HPLC to give tert-butyl (3S)-4-[3-(2,6-dibenzyloxy-3-pyridyl)-1-methyl-indazol-7-yl]-3-methyl-piperazine-1-carboxylate (300 mg, 371.8 μmol, 10.9% yield, 76.8% purity) as yellow oil.

MS (ESI) m/z: 620.4 [M+H]⁺

### Step B. Procedure for Preparation of tert-butyl (3S)-4-[3-(2,6-dioxo-3-piperidyl)-1-methyl-indazol-7-yl]-3-methyl-piperazine-1-carboxylate

To a solution of tert-butyl (3S)-4-[3-(2,6-dibenzyloxy-3-pyridyl)-1-methyl-indazol-7-yl]-3-methyl-piperazine-1-carboxylate (300 mg, 484.07 μmol, 1.0 equiv.) in MeOH (10 mL) was added Pd/C (50 mg, 48.41 μmol, 10% purity, 0.1 equiv.) and Pd(OH)₂ (67.98 mg, 48.41 μmol, 10% purity, 0.1 equiv.) under N₂. The suspension was degassed under vacuum and purged with H₂ several times. The mixture was stirred under H₂ (15 psi) at 25° C. for 16 hours. After the completion of the reaction, the reaction solution was filtered through celite under a moderate N₂ atmosphere and concentrated to give tert-butyl (3S)-4-[3-(2,6-dioxo-3-piperidyl)-1-methyl-indazol-7-yl]-3-methyl-piperazine-1-carboxylate (132 mg, 298.9 μmol, 61.7% yield) as a yellow solid.

MS (ESI) m/z: 442.3 [M+H]⁺

### Step C. Procedure for Preparation of 3-[1-methyl-7-[(2S)-2-methylpiperazin-1-yl]indazol-3-yl]piperidine-2,6-dione

To a solution of tert-butyl (3S)-4-[3-(2,6-dioxo-3-piperidyl)-1-methyl-indazol-7-yl]-3-methyl-piperazine-1-carboxylate (200 mg, 452.98 μmol, 1.0 equiv.) in HCl/dioxane (10 mL) and dioxane (10 mL). The mixture was stirred at 25° C. for 2 hours. The mixture was filtered to give 3-[1-methyl-7-[(2S)-2-methylpiperazin-1-yl]indazol-3-yl]piperidine-2,6-dione (132 mg, 349.4 μmol, 77.1% yield, 90.3% purity) as yellow oil which was used in the next step without further purification.

MS (ESI) m/z: 342.2 [M+H]⁺

### Step D. Procedure for Preparation of tert-butyl 6-[8-(1,3-benzothiazol-2-ylcarbamoyl)-3,4-dihydro-1H-isoquinolin-2-yl]-3-[3-[4-[3-[(3S)-4-[3-(2,6-dioxo-3-piperidyl)-1-methyl-indazol-7-yl]-3-methyl-piperazin-1-yl]propyl]cyclohexoxy]-2-methyl-phenyl]pyridine-2-carboxylate

To a solution of 3-[1-methyl-7-[(2S)-2-methylpiperazin-1-yl]indazol-3-yl]piperidine-2,6-dione (90 mg, 263.62 μmol, 1.0 equiv.) in DCM (5 mL) was added dropwise NaBH(OAc)₃ (167.61 mg, 790.85 μmol, 3.0 equiv.) at 25° C. After addition, the mixture was stirred at this temperature for 0.5 hour, and then tert-butyl 6-[8-(1,3-benzothiazol-2-ylcarbamoyl)-3,4-dihydro-1H-isoquinolin-2-yl]-3-[2-methyl-3-[4-(3-oxopropyl)cyclohexoxy]phenyl]pyridine-2-carboxylate (192.68 mg, 263.62 μmol, 1.0 equiv.) was added dropwise at 0° C. The resulting mixture was stirred at 25° C. for 16 hours. The reaction mixture was concentrated under reduced pressure to remove solvent. The residue was diluted with H₂O (50 mL) and extracted with ethyl acetate (50 mL×3). The combined organic layers were dried over Na₂SO₄, filtered, and concentrated under reduced pressure to give a tert-butyl 6-[8-(1,3-benzothiazol-2-ylcarbamoyl)-3,4-dihydro-1H-isoquinolin-2-yl]-3-[3-[4-[3-[(3S)-4-[3-(2,6-dioxo-3-piperidyl)-1-methyl-indazol-7-yl]-3-methyl-piperazin-1-yl]propyl]cyclohexoxy]-2-methyl-phenyl]pyridine-2-carboxylate (128 mg, 119.9 μmol, 45.5% yield, 99% purity) as a brown solid.

MS (ESI) m/z: 529.1 [M/2+H]⁺

### Step E. Procedure for Preparation of 6-(8-(benzo[d]thiazol-2-ylcarbamoyl)-3,4-dihydroisoquinolin-2(1H)-yl)-3-(3-(((1R,4r)-4-(3-((3S)-4-(3-(2,6-dioxopiperidin-3-yl)-1-methyl-1H-indazol-7-yl)-3-methylpiperazin-1-yl)propyl)cyclohexyl)oxy)-2-methylphenyl)picolinic acid

A solution of tert-butyl 6-[8-(1,3-benzothiazol-2-ylcarbamoyl)-3,4-dihydro-1H-isoquinolin-2-yl]-3-[3-[4-[3-[(3S)-4-[3-(2,6-dioxo-3-piperidyl)-1-methyl-indazol-7-yl]-3-methyl-piperazin-1-yl]propyl]cyclohexoxy]-2-methyl-phenyl]pyridine-2-carboxylate (95 mg, 89.93 μmol, 1.0 equiv.) in TFA (3 mL) and DCM (6 mL) was stirred at 25° C. for 16 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1R,4r)-4-(3-((3S)-4-(3-(2,6-dioxopiperidin-3-yl)-1-methyl-1H-indazol-7-yl)-3-methylpiperazin-1-yl)propyl)cyclohexyl)oxy)-2-methylphenyl)picolinic acid (56.45 mg, 56.44 μmol, 62.75% yield) as a yellow solid.

MS (ESI) m/z: 1001.3 [M+H]⁺

¹H NMR (400 MHz, DMSO-d₆) δ=10.88 (s, 1H), 8.15 (s, 1H), 8.03 (d, J=8.0 Hz, 1H), 7.79 (d, J=8.0 Hz, 1H), 7.62 (d, J=7.6 Hz, 1H), 7.49-7.42 (m, 4H), 7.39-7.33 (m, 2H), 7.24 (d, J=5.6 Hz, 1H), 7.07 (t, J=7.6 Hz, 2H), 6.94 (dd, J=8.8, 12.8 Hz, 2H), 6.62 (d, J=7.6 Hz, 1H), 4.98 (s, 2H), 4.34 (dd, J=5.2, 10.6 Hz, 1H), 4.27 (s, 3H), 4.23-4.17 (m, 1H), 3.91 (t, J=6.0 Hz, 2H), 3.02 (t, J=5.6 Hz, 2H), 2.88-2.84 (m, 1H), 2.81-2.73 (m, 2H), 2.70-2.61 (m, 4H), 2.33 (d, J=1.6 Hz, 3H), 2.29-2.23 (m, 1H), 2.17 (dd, J=5.2, 13.2 Hz, 1H), 2.11-2.05 (m, 2H), 1.94-1.85 (m, 4H), 1.80 (d, J=10.4 Hz, 2H), 1.53-1.46 (m, 2H), 1.40-1.33 (m, 2H), 1.29-1.21 (m, 3H), 1.13-1.04 (m, 2H), 0.86-0.77 (m, 3H).

### Example 76. Preparation of Compound 275a

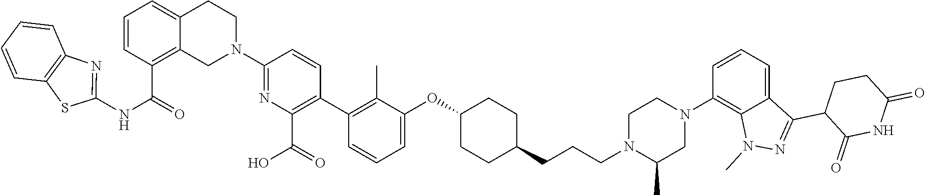 [SMILES: Cc1c(O[C@H]2CC[C@H](CCCN3CCN(c4cccc5c(C6CCC(=O)NC6=O)nn(C)c45)C[C@H]3C)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1S,4r)-4-(3-((2R)-4-(3-(2,6-dioxopiperidin-3-yl)-1-methyl-1H-indazol-7-yl)-2-methylpiperazin-1-yl)propyl)cyclohexyl)oxy)-2-methylphenyl)picolinic acid

### Step A. Procedure for Preparation of tert-butyl (2R)-4-[3-(2,6-dibenzyloxy-3-pyridyl)-1-methyl-indazol-7-yl]-2-methyl-piperazine-1-carboxylate

A mixture of 7-bromo-3-(2,6-dibenzyloxy-3-pyridyl)-1-methyl-indazole (1.5 g, 3.00 mmol, 1 equiv.), tert-butyl (2R)-2-methylpiperazine-1-carboxylate (1.80 g, 8.99 mmol, 3 equiv.), RuPhos (279.77 mg, 599.54 μmol, 0.2 equiv.), Cs₂CO₃ (2.93 g, 8.99 mmol, 3 equiv.), and Pd₂(dba)₃ (274.50 mg, 299.77 μmol, 0.1 equiv.) in toluene (15 mL) was degassed and purged with N₂ three times, and then the mixture was stirred at 110° C. for 16 hours under N₂ atmosphere. The reaction mixture was filtered and concentrated under reduced pressure to give a residue. The residue was purified by flash silica gel chromatography (Eluent of 0-15% ethyl acetate/petroleum ether) to afford tert-butyl (2R)-4-[3-(2,6-dibenzyloxy-3-pyridyl)-1-methyl-indazol-7-yl]-2-methyl-piperazine-1-carboxylate (1 g, 1.4 mmol, 49.5% yield, 92% purity) as a yellow oil.

MS (ESI) m/z: 620.4 [M+H]⁺.

### Step B. Procedure for Preparation of tert-butyl (2R)-4-[3-(2,6-dioxo-3-piperidyl)-1-methyl-indazol-7-yl]-2-methyl-piperazine-1-carboxylate

To a solution of tert-butyl (2R)-4-[3-(2,6-dibenzyloxy-3-pyridyl)-1-methyl-indazol-7-yl]-2-methyl-piperazine-1-carboxylate (1.14 g, 1.84 mmol, 1 equiv.) in THE (10 mL) and EtOH (10 mL) was added Pd/C (300 mg, 1.84 mmol, 10% purity, 1.00 equiv.), Pd(OH)₂ (300 mg, 213.61 μmol, 10% purity, 1.16e-1 equiv.), and AcOH (110.46 mg, 1.84 mmol, 105.20 μL, 1 equiv.) under N₂ atmosphere. The suspension was degassed and purged with H₂ three times. The mixture was stirred under H₂ (15 Psi) at 25° C. for 16 hours. The reaction mixture was filtered, and the filter cake was washed with THF (50 mL). The filtrate was concentrated to afford tert-butyl (2R)-4-[3-(2,6-dioxo-3-piperidyl)-1-methyl-indazol-7-yl]-2-methyl-piperazine-1-carboxylate (900 mg, crude) as a black, brown oil, which was used in the next step without further purification.

### Step C. Procedure for Preparation of 3-[1-methyl-7-[(3R)-3-methylpiperazin-1-yl]indazol-3-yl]piperidine-2,6-dione

To a solution of tert-butyl (2R)-4-[3-(2,6-dioxo-3-piperidyl)-1-methyl-indazol-7-yl]-2-methyl-piperazine-1-carboxylate (900 mg, 2.04 mmol, 1 equiv.) in dioxane (10 mL) was added HCl/EtOAc (4 M, 509.60 uL, 1 equiv.). The mixture was stirred at 25° C. for 2 hours. The reaction mixture was filtered to afford 3-[1-methyl-7-[(3R)-3-methylpiperazin-1-yl]indazol-3-yl]piperidine-2,6-dione (550 mg, crude) as a light-yellow solid, was used in the next step without further purification.

Step D. Procedure for Preparation of tert-butyl 6-[8-(1,3-benzothiazol-2-ylcarbamoyl)-3,4-dihydro-1H-isoquinolin-2-yl]-3-[3-[4-[3-[(2R)-4-[3-(2,6-dioxo-3-piperidyl)-1-methyl-indazol-7-yl]-2-methyl-piperazin-1-yl]propyl]cyclohexoxy]-2-methyl-phenyl]pyridine-2-carboxylate

To a solution of 3-[1-methyl-7-[(3R)-3-methylpiperazin-1-yl]indazol-3-yl]piperidine-2,6-dione (84.08 mg, 246.27 μmol, 1.2 equiv.) in DCM (2 mL) was added tert-butyl 6-[8-(1,3-benzothiazol-2-ylcarbamoyl)-3,4-dihydro-1H-isoquinolin-2-yl]-3-[2-methyl-3-[4-(3-oxopropyl)cyclohexoxy]phenyl]pyridine-2-carboxylate (150 mg, 205.22 μmol, 1 equiv.). The mixture was stirred at 25° C. for 1 hour and then treated with NaBH(OAc)₃ (130.49 mg, 615.67 μmol, 3 equiv.). The mixture was stirred at 25° C. for 16 hours. The reaction mixture was filtered and concentrated under reduced pressure to give a residue. The residue was purified by prep-TLC (SiO2, DCM:MeOH=15:1) to afford tert-butyl 6-[8-(1,3-benzothiazol-2-ylcarbamoyl)-3,4-dihydro-1H-isoquinolin-2-yl]-3-[3-[4-[3-[(2R)-4-[3-(2,6-dioxo-3-piperidyl)-1-methyl-indazol-7-yl]-2-methyl-piperazin-1-yl]propyl]cyclohexoxy]-2-methyl-phenyl]pyridine-2-carboxylate (30 mg, 24.4 μmol, 11.9% yield, 86% purity) as a light yellow solid.

MS (ESI) m/z: 1056.5 [M+H]⁺.

### Step E. Procedure for Preparation of 6-(8-(benzo[d]thiazol-2-ylcarbamoyl)-3,4-dihydroisoquinolin-2(1H)-yl)-3-(3-(((1S,4r)-4-(3-((2R)-4-(3-(2,6-dioxopiperidin-3-yl)-1-methyl-1H-indazol-7-yl)-2-methylpiperazin-1-yl)propyl)cyclohexyl)oxy)-2-methylphenyl)picolinic acid

To a solution of tert-butyl 6-[8-(1,3-benzothiazol-2-ylcarbamoyl)-3,4-dihydro-1H-isoquinolin-2-yl]-3-[3-[4-[3-[(2R)-4-[3-(2,6-dioxo-3-piperidyl)-1-methyl-indazol-7-yl]-2-methyl-piperazin-1-yl]propyl]cyclohexoxy]-2-methyl-phenyl]pyridine-2-carboxylate (30 mg, 28.40 μmol, 1 equiv.) in DCM (0.5 mL) was added TFA (770.00 mg, 6.75 mmol, 0.5 mL, 237.78 equiv.). The mixture was stirred at 25° C. for 16 hours. The reaction mixture was concentrated under reduced pressure to give a residue. The residue was purified by prep-HPLC to afford 6-(8-(benzo[d]thiazol-2-ylcarbamoyl)-3,4-dihydroisoquinolin-2(1H)-yl)-3-(3-(((1S,4r)-4-(3-((2R)-4-(3-(2,6-dioxopiperidin-3-yl)-1-methyl-1H-indazol-7-yl)-2-methylpiperazin-1-yl)propyl)cyclohexyl)oxy)-2-methylphenyl)picolinic acid (18.9 mg, 18.8 μmol, 66.3% yield, 99.6% purity) as a yellow solid.

MS (ESI) m/z: 1000.5 [M+H]⁺.

¹H NMR (400 MHz, DMSO-d₆) δ=10.88 (s, 1H), 8.02 (d, J=7.6 Hz, 1H), 7.78 (d, J=12.6 Hz, 1H), 7.61 (d, J=7.2 Hz, 1H), 7.51-7.30 (m, 6H), 7.10-6.98 (m, 3H), 6.97-6.87 (m, 2H), 6.62 (d, J=7.6 Hz, 1H), 4.97 (s, 2H), 4.37-4.30 (m, 1H), 4.24 (s, 3H), 4.21-4.13 (m, 1H), 3.96-3.85 (m, 2H), 3.16-3.07 (m, 2H), 3.06-2.98 (m, 3H), 2.97-2.90 (m, 1H), 2.88-2.73 (m, 2H), 2.70-2.60 (m, 3H), 2.36-2.28 (m, 2H), 2.20-2.13 (m, 1H), 2.11-2.04 (m, 2H), 1.87 (s, 3H), 1.80 (d, J=11.6 Hz, 2H), 1.54-1.16 (m, 8H), 1.13-1.02 (m, 5H)

### Example 77. Preparation of Compound 276a

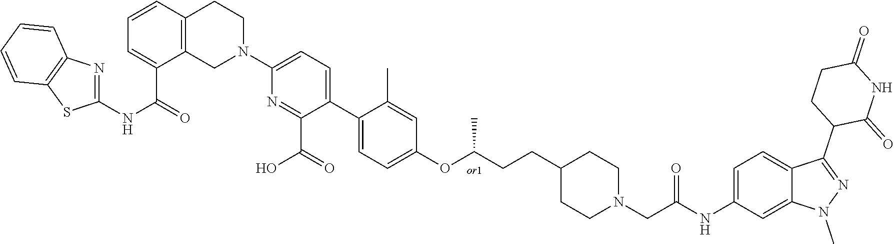 [SMILES: Cc1cc(O[C@H](C)CCC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[4-[(1R)-3-[1-[2-[[3-(2,6-dioxo-3-piperidyl)-1-methyl-indazol-6-yl]amino]-2-oxo-ethyl]-4-piperidyl]-1-methyl-propoxy]-2-methyl-phenyl]pyridine-2-carboxylic acid

### [rel-6-(8-(benzo[d]thiazol-2-ylcarbamoyl)-3,4-dihydroisoquinolin-2(1H)-yl)-3-(4-(((2R)-4-(1-(2-((3-(2,6-dioxopiperidin-3-yl)-1-methyl-1H-indazol-6-yl)amino)-2-oxoethyl)piperidin-4-yl)butan-2-yl)oxy)-2-methylphenyl)picolinic acid]

### Step A. Procedure for Preparation of tert-butyl 4-[3-[methoxy(methyl)amino]-3-oxo-propyl]piperidine-1-carboxylate

To a solution of 3-(1-tert-butoxycarbonyl-4-piperidyl)propanoic acid (2 g, 7.77 mmol, 1 equiv.) in DMF (25 mL) was added HATU (4.43 g, 11.66 mmol, 1.5 equiv.), DIPEA (5.02 g, 38.86 mmol, 6.77 mL, 5 equiv.), and N-methoxymethanamine (1.14 g, 11.66 mmol, 1.5 equiv., HCl). The mixture was stirred at 25° C. for 12 hours. The reaction mixture was filtered and concentrated under reduced pressure to give a residue. The residue was purified by prep-HPLC to give the compound tert-butyl 4-[3-[methoxy(methyl)amino]-3-oxo-propyl]piperidine-1-carboxylate (1.7 g, 5.6 mmol, 72.8% yield) as a yellow oil.

¹H NMR (400 MHz, CDCl₃) δ=4.08 (d, J=13.2 Hz, 2H), 3.69 (s, 3H), 3.19 (s, 3H), 2.77-2.62 (m, 2H), 2.45 (t, J=7.6 Hz, 2H), 1.68 (d, J=12.4 Hz, 2H), 1.64-1.56 (m, 2H), 1.46 (s, 10H), 1.18-1.05 (m, 2H).

### Step B. Procedure for Preparation of tert-butyl 4-(3-oxobutyl)piperidine-1-carboxylate

A mixture of tert-butyl 4-[3-[methoxy(methyl)amino]-3-oxo-propyl]piperidine-1-carboxylate (1.7 g, 5.66 mmol, 1 equiv.) in THE (10 mL) was degassed and purged with N₂ three times, and then MeMgBr (3 M, 11.32 mL, 6 equiv.) was added dropwise to the mixture and stirred at 0° C. for 1 hour under N₂ atmosphere. The reaction mixture was quenched by addition of saturated ammonium chloride (10 mL) at 0° C., and then diluted with water (10 mL) and extracted with CH₂Cl₂ (10 mL×3). The combined organic layers were washed with brine (10 mL×3), dried over anhydrous sodium sulfate, filtered, and concentrated under reduced pressure to give compound tert-butyl 4-(3-oxobutyl)piperidine-1-carboxylate (1.3 g, 5.0 mmol, 89.9% yield) as a white solid.

¹H NMR (400 MHz, DMSO-d₆) δ=3.90 (d, J=12.0 Hz, 2H), 2.69-2.59 (m, 2H), 2.44 (t, J=7.2 Hz, 2H), 2.07 (s, 3H), 1.59 (d, J=12.4 Hz, 3H), 1.38 (s, 9H), 1.35-1.31 (m, 1H), 0.97-0.87 (m, 2H).

### Step C. Procedure for Preparation of tert-butyl 4-(3-hydroxybutyl)piperidine-1-carboxylate

To a solution of tert-butyl 4-(3-oxobutyl)piperidine-1-carboxylate (1.3 g, 5.09 mmol, 1 equiv.) in EtOH (10 mL) was added NaBH₄ (231.13 mg, 6.11 mmol, 1.2 equiv.). The mixture was stirred at 0° C. for 1 hour. The reaction mixture was concentrated under reduced pressure to remove EtOH, and then diluted with EtOAc (10 mL) and NH₄Cl (10 m1) and extracted with EtOAc (10 mL×3). The combined organic layers were washed with brine (10 mL×3), dried over anhydrous sodium sulfate, filtered, and concentrated under reduced pressure to give compound tert-butyl 4-(3-hydroxybutyl)piperidine-1-carboxylate (1.3 g, 4.5 mmol, 89.3% yield, 90% purity) as a yellow oil.

¹H NMR (400 MHz, CDCl₃) δ=4.19-3.96 (m, 2H), 3.85-3.71 (m, 1H), 2.68 (t, J=12.4 Hz, 2H), 1.66 (d, J=12.8 Hz, 2H), 1.53-1.48 (m, 1H), 1.46 (s, 9H), 1.42-1.23 (m, 4H), 1.23-1.17 (m, 3H), 1.17-1.03 (m, 2H).

### Step D. Procedure for Preparation of tert-butyl 4-[3-(4-bromo-3-methyl-phenoxy)butyl]piperidine-1-carboxylate

A mixture of 4-bromo-3-methyl-phenol (1.05 g, 5.60 mmol, 1.2 equiv.), tert-butyl 4-(3-hydroxybutyl)piperidine-1-carboxylate (1.2 g, 4.66 mmol, 1 equiv.), and 2-(tributyl-λ5-phosphanylidene)acetonitrile (1.35 g, 5.60 mmol, 1.2 equiv.) in toluene (10 mL) was degassed and purged with N₂ three times, and then the mixture was stirred at 120° C. for 4 hours under N₂ atmosphere. The reaction mixture was filtered and concentrated under reduced pressure to give a residue. The residue was purified by prep-HPLC to give compound tert-butyl 4-[3-(4-bromo-3-methyl-phenoxy)butyl]piperidine-1-carboxylate (1 g, 2.3 mmol, 49.4% yield, 98.3% purity) as a yellow oil.

MS (ESI) m/z: 369.9 [M-56+H]⁺

¹H NMR (400 MHz, CDCl₃) δ=7.38 (d, J=8.8 Hz, 1H), 6.77 (d, J=2.4 Hz, 1H), 6.63-6.55 (m, 1H), 4.33-4.22 (m, 1H), 4.08 (d, J=13.2 Hz, 2H), 2.67 (t, J=11.6 Hz, 2H), 2.36 (s, 3H), 1.70-1.60 (m, 3H), 1.46 (s, 9H), 1.43-1.19 (m, 7H), 1.15-1.03 (m, 2H).

### Step E. Procedure for Preparation of tert-butyl 4-[(3R)-3-(4-bromo-3-methyl-phenoxy)butyl]piperidine-1-carboxylate

Tert-butyl 4-[3-(4-bromo-3-methyl-phenoxy)butyl]piperidine-1-carboxylate was further separated by SFC to give tert-butyl 4-[(3R)-3-(4-bromo-3-methyl-phenoxy)butyl]piperidine-1-carboxylate (400 mg, 920.7 μmol, 39.2% yield, 98.1% purity) as a yellow oil and tert-butyl 4-[(3S)-3-(4-bromo-3-methyl-phenoxy)butyl]piperidine-1-carboxylate (350 mg, 820.4 μmol, 34.9% yield, 99.9% purity) as a yellow oil.

### Step F. Procedure for Preparation of 4-[(3R)-3-(4-bromo-3-methyl-phenoxy)butyl]piperidine

To a solution of tert-butyl 4-[(3R)-3-(4-bromo-3-methyl-phenoxy)butyl]piperidine-1-carboxylate (400 mg, 938.12 μmol, 1 equiv.) was added HCl/EtOAc (4 M, 234.53 μL, 1 equiv.). The mixture was stirred at 25° C. for 1 hour. The reaction mixture was filtered and concentrated under reduced pressure to give 4-[(3R)-3-(4-bromo-3-methyl-phenoxy)butyl]piperidine (400 mg, crude) as a yellow oil.

Step G. Procedure for Preparation of ethyl 2-[4-[(3R)-3-(4-bromo-3-methyl-phenoxy)butyl]-1-piperidyl]acetate

To a solution of 4-[(3R)-3-(4-bromo-3-methyl-phenoxy)butyl]piperidine (400 mg, 1.23 mmol, 1 equiv.), ethyl 2-bromoacetate (204.74 mg, 1.23 mmol, 135.59 μL, 1 equiv.) in CH₃CN (5 mL) was added K₂CO₃ (508.31 mg, 3.68 mmol, 3 equiv.). The mixture was stirred at 60° C. for 2 hours. The reaction mixture was filtered and concentrated under reduced pressure to give ethyl 2-[4-[(3R)-3-(4-bromo-3-methyl-phenoxy)butyl]-1-piperidyl]acetate (350 mg, crude) was obtained as a yellow oil.

MS (ESI) m/z: 412.9 [M+H]⁺

### Step H. Procedure for Preparation of tert-butyl 6-[8-(1,3-benzothiazol-2-ylcarbamoyl)-3,4-dihydro-1H-isoquinolin-2-yl]-3-[4-[(1R)-3-[1-(2-ethoxy-2-oxo-ethyl)-4-piperidyl]-1-methyl-propoxy]-2-methyl-phenyl]pyridine-2-carboxylate

A mixture of ethyl 2-[4-[(3R)-3-(4-bromo-3-methyl-phenoxy)butyl]-1-piperidyl]acetate (150 mg, 363.76 μmol, 1 equiv.) and tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (222.82 mg, 363.76 μmol, 1 equiv.) in dioxane (3 mL) was added KF (1.5 M, 727.52 μL, 3 equiv.) and Ad₂nBuP Pd G₃ (cataCXium® A Pd G₃) (26.49 mg, 36.38 μmol, 0.1 equiv.). After addition, the mixture was degassed and purged with N₂ for three times, and then the mixture was stirred at 100° C. for 2 hours under N₂ atmosphere. The reaction mixture was filtered and concentrated under reduced pressure to give a residue. The residue was purified by flash silica gel chromatography (Eluent of 0˜45% ethyl acetate/petroleum ether) to give tert-butyl 6-[8-(1,3-benzothiazol-2-ylcarbamoyl)-3,4-dihydro-1H-isoquinolin-2-yl]-3-[4-[(1R)-3-[1-(2-ethoxy-2-oxo-ethyl)-4-piperidyl]-1-methyl-propoxy]-2-methyl-phenyl]pyridine-2-carboxylate (150 mg, 171.0 μmol, 47.0% yield, 93.2% purity) as a yellow solid.

MS (ESI) m/z: 818.3 [M+H]⁺

### Step L Procedure for preparation of 2-[4-[(3R)-3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]butyl]-1-piperidyl]acetic acid

To a solution of tert-butyl 6-[8-(1,3-benzothiazol-2-ylcarbamoyl)-3,4-dihydro-1H-isoquinolin-2-yl]-3-[4-[(1R)-3-[1-(2-ethoxy-2-oxo-ethyl)-4-piperidyl]-1-methyl-propoxy]-2-methyl-phenyl]pyridine-2-carboxylate (150 mg, 183.37 μmol, 1 equiv.) in THE (1 mL) and H₂O (1 mL) was added LiOH·H₂O (21.96 mg, 916.83 μmol, 5 equiv.). The mixture was stirred at 25° C. for 1 hour. The reaction mixture was quenched by HCl (1M, 5 mL) and then filtered and concentrated under reduced pressure to give 2-[4-[(3R)-3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]butyl]-1-piperidyl]acetic acid (140 mg, 177.2 μmol, 96.6% yield) was obtained as a white solid.

MS (ESI) m/z: 790.4 [M+H]⁺

### Step J. Procedure for Preparation of tert-butyl 6-[8-(1,3-benzothiazol-2-ylcarbamoyl)-3,4-dihydro-1H-isoquinolin-2-yl]-3-[4-[(1R)-3-[1-[2-[[3-(2,6-dioxo-3-piperidyl)-1-methyl-indazol-6-yl]amino]-2-oxo-ethyl]-4-piperidyl]-1-methyl-propoxy]-2-methyl-phenyl]pyridine-2-carboxylate

To a solution of 2-[4-[(3R)-3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]butyl]-1-piperidyl]acetic acid (140 mg, 177.22 μmol, 1 equiv.) and 3-(6-amino-1-methyl-indazol-3-yl)piperidine-2,6-dione (54.93 mg, 212.66 μmol, 1.2 equiv.) in pyridine (1 mL) was added EDCI (50.96 mg, 265.83 μmol, 1.5 equiv.). The mixture was stirred at 25° C. for 1 hour. The reaction mixture was quenched by addition of water (10 mL), and then filtered and concentrated under reduced pressure to give tert-butyl 6-[8-(1,3-benzothiazol-2-ylcarbamoyl)-3,4-dihydro-1H-isoquinolin-2-yl]-3-[4-[(1R)-3-[1-[2-[[3-(2,6-dioxo-3-piperidyl)-1-methyl-indazol-6-yl]amino]-2-oxo-ethyl]-4-piperidyl]-1-methyl-propoxy]-2-methyl-phenyl]pyridine-2-carboxylate (150 mg, 145.6 μmol, 82.1% yield) as a red solid.

MS (ESI) m/z: 1030.4 [M+H]⁺

### Step K. Procedure for Preparation of 6-[8-(1,3-benzothiazol-2-ylcarbamoyl)-3,4-dihydro-1H-isoquinolin-2-yl]-3-[4-[(1R)-3-[1-[2-[[3-(2,6-dioxo-3-piperidyl)-1-methyl-indazol-6-yl]amino]-2-oxo-ethyl]-4-piperidyl]-1-methyl-propoxy]-2-methyl-phenyl]pyridine-2-carboxylic acid [rel-6-(8-(benzo[d]thiazol-2-ylcarbamoyl)-3,4-dihydroisoquinolin-2(1H)-yl)-3-(4-(((2R)-4-(1-(2-((3-(2,6-dioxopiperidin-3-yl)-1-methyl-1H-indazol-6-yl)amino)-2-oxoethyl)piperidin-4-yl)butan-2-yl)oxy)-2-methylphenyl)picolinic acid]

To a solution of tert-butyl 6-[8-(1,3-benzothiazol-2-ylcarbamoyl)-3,4-dihydro-1H-isoquinolin-2-yl]-3-[4-[(1R)-3-[1-[2-[[3-(2,6-dioxo-3-piperidyl)-1-methyl-indazol-6-yl]amino]-2-oxo-ethyl]-4-piperidyl]-1-methyl-propoxy]-2-methyl-phenyl]pyridine-2-carboxylate (150 mg, 145.60 μmol, 1 equiv.) in DCM (2 mL) was added TFA (1.15 g, 10.13 mmol, 750.00 μL, 69.57 equiv.). The mixture was stirred at 25° C. for 12 hours. The reaction mixture was filtered and concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[4-[(1R)-3-[1-[2-[[3-(2,6-dioxo-3-piperidyl)-1-methyl-indazol-6-yl]amino]-2-oxo-ethyl]-4-piperidyl]-1-methyl-propoxy]-2-methyl-phenyl]pyridine-2-carboxylic acid [rel-6-(8-(benzo[d]thiazol-2-ylcarbamoyl)-3,4-dihydroisoquinolin-2(1H)-yl)-3-(4-(((2R)-4-(1-(2-((3-(2,6-dioxopiperidin-3-yl)-1-methyl-1H-indazol-6-yl)amino)-2-oxoethyl)piperidin-4-yl)butan-2-yl)oxy)-2-methylphenyl)picolinic acid](91.9 mg, 93 μmol, 64.2% yield, 99.1% purity) as a white solid.

MS (ESI) m/z: 974.3 [M+H]⁺

¹H NMR (400 MHz, DMSO-d₆) δ=10.87 (s, 1H), 9.82 (s, 1H), 8.15 (s, 1H), 8.09-7.99 (m, 2H), 7.79 (d, J=8.0 Hz, 1H), 7.63 (d, J=8.8 Hz, 2H), 7.50-7.42 (m, 3H), 7.40-7.31 (m, 2H), 7.25-7.19 (m, 1H), 6.92 (t, J=9.2 Hz, 2H), 6.76 (d, J=2.0 Hz, 1H), 6.73-6.64 (m, 1H), 4.97 (s, 2H), 4.43-4.36 (m, 1H), 4.35-4.29 (m, 1H), 3.97-3.87 (m, 5H), 3.13 (s, 2H), 3.02 (t, J=5.2 Hz, 2H), 2.87 (d, J=10.4 Hz, 2H), 2.67-2.59 (m, 2H), 2.37-2.29 (m, 1H), 2.21-2.09 (m, 3H), 2.02 (s, 3H), 1.72-1.62 (m, 3H), 1.61-1.51 (m, 1H), 1.43-1.34 (m, 1H), 1.33-1.20 (m, 7H).

### Example 78. Preparation of Compound 276b

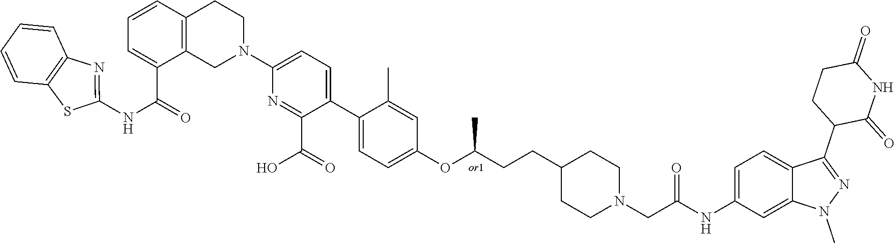 [SMILES: Cc1cc(O[C@@H](C)CCC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[4-[(1S)-3-[1-[2-[[3-(2,6-dioxo-3-piperidyl)-1-methyl-indazol-6-yl]amino]-2-oxo-ethyl]-4-piperidyl]-1-methyl-propoxy]-2-methyl-phenyl]pyridine-2-carboxylic acid

### [rel-6-(8-(benzo[d]thiazol-2-ylcarbamoyl)-3,4-dihydroisoquinolin-2(1H)-yl)-3-(4-(((2S)-4-(1-(2-((3-(2,6-dioxopiperidin-3-yl)-1-methyl-1H-indazol-6-yl)amino)-2-oxoethyl)piperidin-4-yl)butan-2-yl)oxy)-2-methylphenyl)picolinic acid]

### Step A. Procedure for Preparation of 4-[(3S)-3-(4-bromo-3-methyl-phenoxy)butyl]piperidine

A solution of tert-butyl 4-[(3S)-3-(4-bromo-3-methyl-phenoxy)butyl]piperidine-1-carboxylate (350.00 mg, 820.85 μmol, 1 equiv.) in HCl/EtOAc (4 mL) was stirred at 25° C. for 1 hour. The reaction mixture was filtered and concentrated under reduced pressure to give 4-[(3S)-3-(4-bromo-3-methyl-phenoxy)butyl]piperidine (350 mg, crude) as a yellow oil.

MS (ESI) m/z: 326.0 [M+H]⁺

### Step B. Procedure for Preparation of ethyl 2-[4-[(3S)-3-(4-bromo-3-methyl-phenoxy)butyl]-1-piperidyl]acetate

To a solution of 4-[(3S)-3-(4-bromo-3-methyl-phenoxy)butyl]piperidine (350 mg, 1.07 mmol, 1 equiv.), ethyl 2-bromoacetate (179.15 mg, 1.07 mmol, 118.64 μL, 1 equiv.) in CH₃CN (4 mL) was added K₂CO₃ (444.79 mg, 3.22 mmol, 3 equiv.). The mixture was stirred at 60° C. for 2 hours. The reaction mixture was filtered and concentrated under reduced pressure to give ethyl 2-[4-[(3S)-3-(4-bromo-3-methyl-phenoxy)butyl]-1-piperidyl]acetate (340 mg, crude) as a yellow oil.

MS (ESI) m/z: 412.0 [M+H]⁺

### Step C. Procedure for Preparation of tert-butyl 6-[8-(1,3-benzothiazol-2-ylcarbamoyl)-3,4-dihydro-1H-isoquinolin-2-yl]-3-[4-[(1S)-3-[1-(2-ethoxy-2-oxo-ethyl)-4-piperidyl]-1-methyl-propoxy]-2-methyl-phenyl]pyridine-2-carboxylate

A mixture of ethyl 2-[4-[(3S)-3-(4-bromo-3-methyl-phenoxy)butyl]-1-piperidyl]acetate (150.00 mg, 363.76 μmol, 1 equiv.) and tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (222.82 mg, 363.76 μmol, 1 equiv.) in dioxane (3 mL) was added KF (1.5 M, 727.52 μL, 3 equiv.) and Ad₂nBuP Pd G₃(cataCXium® A Pd G₃) (26.49 mg, 36.38 μmol, 0.1 equiv.). After addition, the mixture was degassed and purged with N₂ three times, and then the mixture was stirred at 100° C. for 2 hours under N₂ atmosphere. The reaction mixture was filtered and concentrated under reduced pressure to give a residue. The residue was purified by flash silica gel chromatography (Eluent of 0˜40% ethyl acetate/petroleum ether) to give compound tert-butyl 6-[8-(1,3-benzothiazol-2-ylcarbamoyl)-3,4-dihydro-1H-isoquinolin-2-yl]-3-[4-[(1S)-3-[1-(2-ethoxy-2-oxo-ethyl)-4-piperidyl]-1-methyl-propoxy]-2-methyl-phenyl]pyridine-2-carboxylate (180 mg, 210.7 μmol, 57.9% yield, 95.7% purity) as a white solid.

MS (ESI) m/z: 818.8 [M+H]⁺

### Step D. Procedure for Preparation of 2-[4-[(3S)-3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]butyl]-1-piperidyl]acetic acid

To a solution of tert-butyl 6-[8-(1,3-benzothiazol-2-ylcarbamoyl)-3,4-dihydro-1H-isoquinolin-2-yl]-3-[4-[(1S)-3-[1-(2-ethoxy-2-oxo-ethyl)-4-piperidyl]-1-methyl-propoxy]-2-methyl-phenyl]pyridine-2-carboxylate (150 mg, 183.37 μmol, 1 equiv.) in THE (1 mL) and H₂O (1 mL) was added LiOH (21.96 mg, 916.83 μmol, 5 equiv.). The mixture was stirred at 25° C. for 1 hour. The reaction mixture was quenched by HCl 1M (5 mL), and then filtered and concentrated under reduced pressure to give 2-[4-[(3S)-3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]butyl]-1-piperidyl]acetic acid (130 mg, 164.5 μmol, 89.7% yield) as a white solid.

MS (ESI) m/z: 790.3 [M+H]⁺

### Step E. Procedure for Preparation of tert-butyl 6-[8-(1,3-benzothiazol-2-ylcarbamoyl)-3,4-dihydro-1H-isoquinolin-2-yl]-3-[4-[(1S)-3-[1-[2-[[3-(2,6-dioxo-3-piperidyl)-1-methyl-indazol-6-yl]amino]-2-oxo-ethyl]-4-piperidyl]-1-methyl-propoxy]-2-methyl-phenyl]pyridine-2-carboxylate

To a solution of 2-[4-[(3S)-3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]butyl]-1-piperidyl]acetic acid (130.00 mg, 164.56 μmol, 1 equiv.) and 3-(6-amino-1-methyl-indazol-3-yl)piperidine-2,6-dione (51.00 mg, 197.47 μmol, 1.2 equiv.) in pyridine (1 mL) was added EDCI (47.32 mg, 246.84 μmol, 1.5 equiv.). The mixture was stirred at 25° C. for 1 hour. The reaction mixture was quenched by addition of water (10 mL), and then filtered and concentrated under reduced pressure to give tert-butyl 6-[8-(1,3-benzothiazol-2-ylcarbamoyl)-3,4-dihydro-1H-isoquinolin-2-yl]-3-[4-[(1S)-3-[1-[2-[[3-(2,6-dioxo-3-piperidyl)-1-methyl-indazol-6-yl]amino]-2-oxo-ethyl]-4-piperidyl]-1-methyl-propoxy]-2-methyl-phenyl]pyridine-2-carboxylate (150 mg, 145.6 μmol, 88.4% yield) as a red solid.

MS (ESI) m/z: 1030.4 [M+H]⁺

### Step F. Procedure for Preparation of 6-[8-(1,3-benzothiazol-2-ylcarbamoyl)-3,4-dihydro-1H-isoquinolin-2-yl]-3-[4-[(1S)-3-[1-[2-[[3-(2,6-dioxo-3-piperidyl)-1-methyl-indazol-6-yl]amino]-2-oxo-ethyl]-4-piperidyl]-1-methyl-propoxy]-2-methyl-phenyl]pyridine-2-carboxylic acid [rel-6-(8-(benzo[d]thiazol-2-ylcarbamoyl)-3,4-dihydroisoquinolin-2(1H)-yl)-3-(4-(((2S)-4-(1-(2-((3-(2,6-dioxopiperidin-3-yl)-1-methyl-1H-indazol-6-yl)amino)-2-oxoethyl)piperidin-4-yl)butan-2-yl)oxy)-2-methylphenyl)picolinic acid]

To a solution of tert-butyl 6-[8-(1,3-benzothiazol-2-ylcarbamoyl)-3,4-dihydro-1H-isoquinolin-2-yl]-3-[4-[(1S)-3-[1-[2-[[3-(2,6-dioxo-3-piperidyl)-1-methyl-indazol-6-yl]amino]-2-oxo-ethyl]-4-piperidyl]-1-methyl-propoxy]-2-methyl-phenyl]pyridine-2-carboxylate (150.00 mg, 145.60 μmol, 1 equiv.) in DCM (2 mL) was added TFA (1.15 g, 10.13 mmol, 750.00 μL, 69.57 equiv.). The mixture was stirred at 25° C. for 12 hours. The reaction mixture was filtered and concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[4-[(1S)-3-[1-[2-[[3-(2,6-dioxo-3-piperidyl)-1-methyl-indazol-6-yl]amino]-2-oxo-ethyl]-4-piperidyl]-1-methyl-propoxy]-2-methyl-phenyl]pyridine-2-carboxylic acid [rel-6-(8-(benzo[d]thiazol-2-ylcarbamoyl)-3,4-dihydroisoquinolin-2(1H)-yl)-3-(4-(((2S)-4-(1-(2-((3-(2,6-dioxopiperidin-3-yl)-1-methyl-1H-indazol-6-yl)amino)-2-oxoethyl)piperidin-4-yl)butan-2-yl)oxy)-2-methylphenyl)picolinic acid](70.2 mg, 71.2 μmol, 48.9% yield, 98.8% purity) as a yellow solid.

MS (ESI) m/z: 974.4 [M+H]⁺

¹H NMR (400 MHz, DMSO-d₆) δ=10.87 (s, 1H), 9.82 (s, 1H), 8.15 (s, 1H), 8.07-8.00 (m, 2H), 7.79 (d, J=8.0 Hz, 1H), 7.62 (d, J=8.4 Hz, 2H), 7.50-7.41 (m, 3H), 7.40-7.31 (m, 2H), 7.25-7.18 (m, 1H), 6.96-6.88 (m, 2H), 6.76 (d, J=2.0 Hz, 1H), 6.72-6.65 (m, 1H), 4.97 (s, 2H), 4.43-4.36 (m, 1H), 4.35-4.29 (m, 1H), 3.95-3.87 (m, 5H), 3.13 (s, 2H), 3.02 (t, J=5.6 Hz, 2H), 2.87 (d, J=10.4 Hz, 2H), 2.68-2.60 (m, 2H), 2.37-2.30 (m, 1H), 2.20-2.10 (m, 3H), 2.02 (s, 3H), 1.66 (d, J=8.0 Hz, 3H), 1.61-1.52 (m, 1H), 1.43-1.35 (m, 1H), 1.32-1.21 (m, 7H).

### Example 79. Preparation of Compound 279

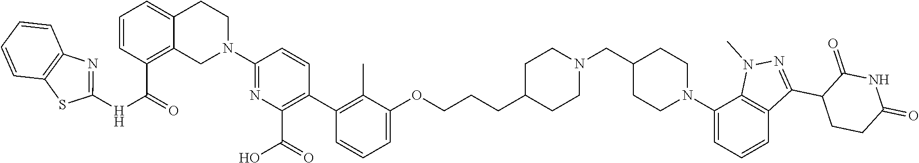

### 6-[8-(1,3-benzothiazol-2-ylcarbamoyl)-3,4-dihydro-1H-isoquinolin-2-yl]-3-[3-[3-[1-[[1-[3-(2,6-dioxo-3-piperidyl)-1-methyl-indazol-7-yl]-4-piperidyl]methyl]-4-piperidyl]propoxy]-2-methyl-phenyl]pyridine-2-carboxylic acid

### Step A. Procedure for Preparation of 3-(2,6-dibenzyloxy-3-pyridyl)-7-[4-(dimethoxymethyl)-1-piperidyl]-1-methyl-indazole

A mixture of 7-bromo-3-(2,6-dibenzyloxy-3-pyridyl)-1-methyl-indazole (1 g, 2.00 mmol, 1 equiv.), 4-(dimethoxymethyl)piperidine (477.31 mg, 3.00 mmol, 1.5 equiv.), 1,3-bis[2,6-bis(1-propylbutyl)phenyl]-4,5-dichloro-2H-imidazol-1-ium-2-ide; 3-chloropyridine; dichloropalladium (194.40 mg, 199.85 μmol, 0.1 equiv.), and Cs₂CO₃ (1.95 g, 6.00 mmol, 3 equiv.) in 2-methyl-2-butanol (15 mL) was degassed and purged with N₂ three times, and then the mixture was stirred at 110° C. for 3 hours under N₂ atmosphere. The reaction mixture was filtered and concentrated under reduced pressure to remove solvent. The residue was purified by flash silica gel chromatography (Eluent of 0˜20% ethyl acetate/petroleum ether). The compound 3-(2,6-dibenzyloxy-3-pyridyl)-7-[4-(dimethoxymethyl)-1-piperidyl]-1-methyl-indazole (360 mg, 622.0 μmol, 31.1% yield) was obtained as a yellow solid.

MS (ESI) m/z: 579.5 [M+H]+.

### Step B. Procedure for Preparation of 3-[7-[4-(dimethoxymethyl)-1-piperidyl]-1-methyl-indazol-3-yl]piperidine-2,6-dione

A mixture of 3-(2,6-dibenzyloxy-3-pyridyl)-7-[4-(dimethoxymethyl)-1-piperidyl]-1-methyl-indazole (360 mg, 622.08 μmol, 1 equiv.), Pd/C (150 mg, 622.08 μmol, 10% purity, 1 equiv.), Pd(OH)₂ (150 mg, 106.81 μmol, 10% purity, 0.172 equiv.) and AcOH (112.07 mg, 1.87 mmol, 106.74 μL, 3 equiv.) in EtOH (2.5 mL) and THE (2.5 mL) was degassed and purged with H₂ three times, and then the mixture was stirred at 25° C. for 12 hours under H₂ (15 Psi) atmosphere. The mixture was filtered with THE (30 mL), and the filtrate was concentrated under reduced pressure to give 3-[7-[4-(dimethoxymethyl)-1-piperidyl]-1-methyl-indazol-3-yl]piperidine-2,6-dione (330 mg, crude) as a black solid, which was used in the next step without further purification.

MS (ESI) m/z: 401.3 [M+H]⁺.

### Step C. Procedure for Preparation of 1-[3-(2,6-dioxo-3-piperidyl)-1-methyl-indazol-7-yl]piperidine-4-carbaldehyde

A mixture of 3-[7-[4-(dimethoxymethyl)-1-piperidyl]-1-methyl-indazol-3-yl]piperidine-2,6-dione (330 mg, 824.03 μmol, 1 equiv.) in HCOOH (3.5 mL) was degassed and purged with N₂ three times, and then the mixture was stirred at 80° C. for 2 hours under N₂ atmosphere. The reaction mixture was concentrated under reduced pressure to give 1-[3-(2,6-dioxo-3-piperidyl)-1-methyl-indazol-7-yl]piperidine-4-carbaldehyde (220 mg, crude) as a brown solid.

MS (ESI) m/z: 355.2 [M+H]⁺.

### Step D. Procedure for Preparation of 6-[8-(1,3-benzothiazol-2-ylcarbamoyl)-3,4-dihydro-1H-isoquinolin-2-yl]-3-[3-[3-[1-[[1-[3-(2,6-dioxo-3-piperidyl)-1-methyl-indazol-7-yl]-4-piperidyl]methyl]-4-piperidyl]propoxy]-2-methyl-phenyl]pyridine-2-carboxylic acid

A solution of 6-[8-(1,3-benzothiazol-2-ylcarbamoyl)-3,4-dihydro-1H-isoquinolin-2-yl]-3-[2-methyl-3-[3-(4-piperidyl)propoxy]phenyl]pyridine-2-carboxylic acid (150 mg, 226.65 μmol, 1 equiv.) and 1-[3-(2,6-dioxo-3-piperidyl)-1-methyl-indazol-7-yl]piperidine-4-carbaldehyde (240.98 mg, 679.95 μmol, 3 equiv.) in DCM (15 mL) was stirred at 25° C. for 11 h. To the mixture was then added NaBH(OAc)₃ (144.11 mg, 679.95 μmol, 3 equiv.). The mixture was stirred at 25° C. for 1 h. The reaction mixture was quenched by addition H₂O (30 mL) and extracted with DCM (30 mL×2). The combined organic layers were concentrated under reduced pressure to give a residue. The residue was purified by prep-HPLC. The compound 6-[8-(1,3-benzothiazol-2-ylcarbamoyl)-3,4-dihydro-1H-isoquinolin-2-yl]-3-[3-[3-[1-[[1-[3-(2,6-dioxo-3-piperidyl)-1-methyl-indazol-7-yl]-4-piperidyl]methyl]-4-piperidyl]propoxy]-2-methyl-phenyl]pyridine-2-carboxylic acid (23.9 mg, 21.6 μmol, 9.5% yield, 90.5% purity) was obtained as an off-white solid.

¹H NMR (400 MHz, DMSO-d₆) δ=10.87 (s, 1H), 8.01 (d, J=7.6 Hz, 1H), 7.78 (d, J=8.4 Hz, 1H), 7.62 (d, J=7.6 Hz, 1H), 7.48-7.40 (m, 3H), 7.39-7.31 (m, 3H), 7.11-7.05 (m, 1H), 7.03-6.98 (m, 2H), 6.95-6.90 (m, 1H), 6.87 (d, J=7.6 Hz, 1H), 6.64 (d, J=7.6 Hz, 1H), 4.97 (s, 2H), 4.32 (dd, J=5.2, 9.6 Hz, 1H), 4.23 (s, 3H), 3.97-3.89 (m, 4H), 3.02 (t, J=5.6 Hz, 2H), 2.90-2.83 (m, 3H), 2.65-2.62 (m, 2H), 2.30-2.12 (m, 5H), 1.92-1.82 (m, 7H), 1.78-1.62 (m, 6H), 1.38-1.23 (m, 6H), 1.20-1.10 (m, 2H)

### Example 80. Preparation of Compound 280

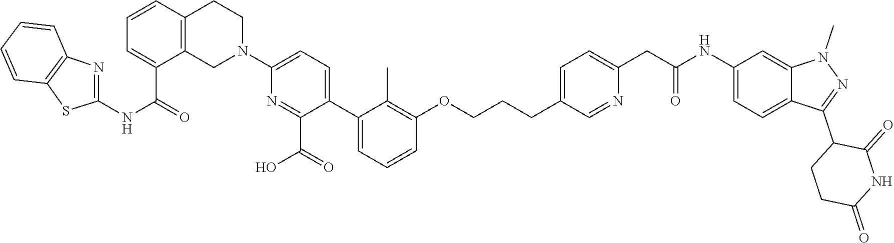 [SMILES: Cc1c(OCCCc2ccc(CC(=O)Nc3ccc4c(C5CCC(=O)NC5=O)nn(C)c4c3)nc2)cccc1-c1ccc(N2CCc3cccc(C(=O)Nc4nc5ccccc5s4)c3C2)nc1C(=O)O]

### 6-[8-(1, 3-benzothiazol-2-ylcarbamoyl)-3, 4-dihydro-1H-isoquinolin-2-yl]-3-[3-[3-[6-[2-[[3-(2, 6-dioxo-3-piperidyl)-1-methyl-indazol-6-yl]amino]-2-oxo-ethyl]-3-pyridyl]propoxy]-2-methyl-phenyl]pyridine-2-carboxylic acid

### Step A. Procedure for Preparation of methyl 2-(5-bromo-2-pyridyl) acetate

To a solution of 2-(5-bromo-2-pyridyl) acetic acid (2.00 g, 9.26 mmol, 1 equiv.) in MeOH (20 mL) was added SOCl₂ (2.20 g, 18.52 mmol, 1.34 mL, 2 equiv.) at 0° C. The mixture was stirred at 25° C. for 3 hours. The reaction mixture was concentrated under reduced pressure to remove MeOH. The residue was diluted with water (20 mL) and extracted with DCM (20 mL×3). The combined organic layers were washed with NaHCO₃ (30 mL×1), dried over Na₂SO₄, filtered, and concentrated under reduced pressure to give methyl 2-(5-bromo-2-pyridyl) acetate (2.1 g, crude) as a brown oil.

¹H NMR (400 MHz, CCDCl₃-d) δ=8.62 (d, J=2.4 Hz, 1H), 7.80 (dd, J=2.4, 8.4 Hz, 1H), 7.22 (d, J=8.4 Hz, 1H), 3.83 (s, 2H), 3.73 (s, 3H)

### Step B. Procedure for Preparation of methyl 2-[5-[3-[tert-butyl (dimethyl) silyl]oxypropyl]-2-pyridyl]acetate

To an 15 mL vial equipped with a stir bar was added methyl 2-(5-bromo-2-pyridyl) acetate (950 mg, 4.13 mmol, 1 equiv.), 3-bromopropoxy-tert-butyl-dimethyl-silane (1.36 g, 5.37 mmol, 1.3 equiv.), bis[3, 5-difluoro-2-[5-(trifluoromethyl)-2-pyridyl]phenyl]iridium (1+); 4-tert-butyl-2-(4-tert-butyl-2-pyridyl) pyridine; hexafluorophosphate (46.33 mg, 41.29 μmol, 0.01 equiv.), NiCl₂. Dtbbpy (24.65 mg, 61.94 μmol, 0.015 equiv.), TTMSS (1.03 g, 4.13 mmol, 1.27 mL, 1 equiv.), and Na₂CO₃ (875.34 mg, 8.26 mmol, 2 equiv.) in DME (10 mL). The vial was sealed and placed under nitrogen was added. The reaction was stirred and irradiated with a 10 W blue LED lamp (3 cm away), with cooling water to keep the reaction temperature at 25° C. for 14 hours. The reaction mixture was concentrated and the residue was purified by flash silica gel chromatography (Eluent of 0˜10% ethyl acetate/petroleum ether). The compound methyl 2-[5-[3-[tert-butyl (dimethyl) silyl]oxypropyl]-2-pyridyl]acetate (730 mg, 2.1 mmol, 51.3% yield, 94% purity) was obtained as a yellow oil.

MS (ESI) m/z: 324.2 [M+H]⁺.

¹H NMR (400 MHz, CDCl₃-d) δ=8.40 (d, J=2.0 Hz, 1H), 7.50 (dd, J=2.4, 8.0 Hz, 1H), 7.22 (d, J=8.0 Hz, 1H), 3.83 (s, 2H), 3.73 (s, 3H), 3.64 (t, J=6.0 Hz, 2H), 2.73-2.65 (m, 2H), 1.84-1.79 (m, 2H), 0.91 (s, 8H), 0.08-0.02 (m, 6H)

### Step C. Procedure for Preparation of methyl 2-[5-(3-hydroxypropyl)-2-pyridyl]acetate

To a solution of methyl 2-[5-[3-[tert-butyl (dimethyl) silyl]oxypropyl]-2-pyridyl]acetate (370 mg, 1.14 mmol, 1 equiv.) in THE (1.5 mL) was added pyridine; hydrofluoride (1.65 g, 11.65 mmol, 1.5 mL, 70% purity, 10.19 equiv.) at 25° C. The reaction mixture was stirred at 25° C. for 1 hour. The reaction mixture was diluted with water (5 mL) and extracted with EtOAc (10 mL×5). The combined organic layers were dried over Na₂SO₄, filtered, and concentrated under reduced pressure to give a residue. The compound methyl 2-[5-(3-hydroxypropyl)-2-pyridyl]acetate (200 mg, crude) was obtained as a yellow oil.

### Step D. Procedure for Preparation of methyl 2-[5-[3-(3-bromo-2-methyl-phenoxy) propyl]-2-pyridyl]acetate

To a solution of 3-bromo-2-methyl-phenol (196.65 mg, 1.05 mmol, 1.1 equiv.) in toluene (4 mL) was added methyl 2-[5-(3-hydroxypropyl)-2-pyridyl]acetate (200 mg, 955.84 μmol, 1 equiv.) and 2-(tributyl-λ⁵-phosphanylidene) acetonitrile (299.90 mg, 1.24 mmol, 1.3 equiv.) at 25° C. The reaction mixture was degassed and purged with N₂ three times, and then the mixture was stirred at 120° C. for 16 hours under N₂ atmosphere. The reaction mixture was filtered and concentrated under reduced pressure to give a residue. The residue was purified by flash silica gel chromatography (Eluent of 0˜50% ethyl acetate/petroleum ether). The compound methyl 2-[5-[3-(3-bromo-2-methyl-phenoxy) propyl]-2-pyridyl]acetate (190 mg, 497.2 μmol, 52.0% yield, 99% purity) was obtained as a yellow oil.

MS (ESI) m/z: 377.9 [M+H]⁺.

¹H NMR (400 MHz, CDCl₃-d) δ=8.43 (d, J=1.6 Hz, 1H), 7.52 (dd, J=2.0, 8.0 Hz, 1H), 7.24 (d, J=8.0 Hz, 1H), 7.16 (d, J=8.0 Hz, 1H), 6.99 (t, J=8.0 Hz, 1H), 6.73 (d, J=8.4 Hz, 1H), 3.97 (t, J=6.0 Hz, 2H), 3.84 (s, 2H), 3.73 (s, 3H), 2.84 (t, J=7.6 Hz, 2H), 2.34 (s, 3H), 2.18-2.09 (m, 2H)

### Step E. Procedure for Preparation of tert-butyl 6-[8-(1, 3-benzothiazol-2-ylcarbamoyl)-3, 4-dihydro-1H-isoquinolin-2-yl]-3-[3-[3-[6-(2-methoxy-2-oxo-ethyl)-3-pyridyl]propoxy]-2-methyl-phenyl]pyridine-2-carboxylate

Tert-butyl 6-[8-(1, 3-benzothiazol-2-ylcarbamoyl)-3, 4-dihydro-1H-isoquinolin-2-yl]-3-(4, 4, 5, 5-tetramethyl-1, 3, 2-dioxaborolan-2-yl) pyridine-2-carboxylate (267.38 mg, 436.50 μmol, 1.1 equiv.), methyl 2-[5-[3-(3-bromo-2-methyl-phenoxy) propyl]-2-pyridyl]acetate (190 mg, 396.82 mol, 79% purity, 1 equiv.), KF (69.17 mg, 1.19 mmol, 27.89 μL, 3 equiv.), and [2-(2-aminophenyl) phenyl]palladium (1+); bis (1-adamantyl)-butyl-phosphane; methanesulfonate (57.80 mg, 79.36 mol, 0.2 equiv.) were taken up into a microwave tube in dioxane (2 mL) and H₂O (0.2 mL). The sealed tube was heated at 100° C. for 1 hour under microwave. The reaction mixture was filtered and concentrated under reduced pressure to give a residue. The residue was purified by flash silica gel chromatography (Eluent of 0˜30% ethyl acetate/petroleum ether). The compound tert-butyl 6-[8-(1, 3-benzothiazol-2-ylcarbamoyl)-3, 4-dihydro-1H-isoquinolin-2-yl]-3-[3-[3-[6-(2-methoxy-2-oxo-ethyl)-3-pyridyl]propoxy]-2-methyl-phenyl]pyridine-2-carboxylate (130 mg, 97.8 μmol, 24.6% yield, 59% purity) was obtained as a yellow solid.

MS (ESI) m/z: 784.5 [M+H]⁺.

### Step F. Procedure for Preparation of 2-[5-[3-[3-[6-[8-(1, 3-benzothiazol-2-ylcarbamoyl)-3, 4-dihydro-1H-isoquinolin-2-yl]-2-tert-butoxycarbonyl-3-pyridyl]-2-methyl-phenoxy]propyl]-2-pyridyl]acetic acid

To a solution of tert-butyl 6-[8-(1, 3-benzothiazol-2-ylcarbamoyl)-3, 4-dihydro-1H-isoquinolin-2-yl]-3-[3-[3-[6-(2-methoxy-2-oxo-ethyl)-3-pyridyl]propoxy]-2-methyl-phenyl]pyridine-2-carboxylate (130 mg, 97.84 μmol, 59% purity, 1 equiv.) in THE (1 mL) and H₂O (0.1 mL) was added LiOH·H₂O (12.32 mg, 293.52 μmol, 3 equiv.). The mixture was stirred at 25° C. for 16 hours. The reaction mixture was concentrated under reduced pressure to give a residue. The residue was treated with water (4 mL). The pH was adjusted to around 3 by progressively adding diluted HCl. The mixture was filtered and concentrated to give a residue. The compound 2-[5-[3-[3-[6-[8-(1, 3-benzothiazol-2-ylcarbamoyl)-3, 4-dihydro-1H-isoquinolin-2-yl]-2-tert-butoxycarbonyl-3-pyridyl]-2-methyl-phenoxy]propyl]-2-pyridyl]acetic acid (168 mg, crude) was obtained as a yellow solid.

MS (ESI) m/z: 770.5 [M+H]⁺.

### Step G. Procedure for Preparation of tert-butyl 6-[8-(1, 3-benzothiazol-2-ylcarbamoyl)-3, 4-dihydro-1H-isoquinolin-2-yl]-3-[3-[3-[6-[2-[[3-(2, 6-dioxo-3-piperidyl)-1-methyl-indazol-6-yl]amino]-2-oxo-ethyl]-3-pyridyl]propoxy]-2-methyl-phenyl]pyridine-2-carboxylate

To a solution of 2-[5-[3-[3-[6-[8-(1, 3-benzothiazol-2-ylcarbamoyl)-3, 4-dihydro-1H-isoquinolin-2-yl]-2-tert-butoxycarbonyl-3-pyridyl]-2-methyl-phenoxy]propyl]-2-pyridyl]acetic acid (160 mg, 207.82 μmol, 1 equiv.) in pyridine (0.5 mL) was added EDCI (47.81 mg, 249.38 μmol, 1.2 equiv.) and 3-(6-amino-1-methyl-indazol-3-yl) piperidine-2, 6-dione (64.41 mg, 249.38 μmol, 1.2 equiv.) at 25° C. The reaction mixture was stirred at 25° C. for 16 hours. The mixture was triturated with water (5 mL) at 25° C. for 10 min. The mixture was filtered to give the compound tert-butyl 6-[8-(1, 3-benzothiazol-2-ylcarbamoyl)-3, 4-dihydro-1H-isoquinolin-2-yl]-3-[3-[3-[6-[2-[[3-(2, 6-dioxo-3-piperidyl)-1-methyl-indazol-6-yl]amino]-2-oxo-ethyl]-3-pyridyl]propoxy]-2-methyl-phenyl]pyridine-2-carboxylate (80 mg, 38.9 μmol, 18.7% yield, 49.2% purity) as a yellow solid.

MS (ESI) m/z: 1010.3 [M+H]⁺.

### Step H. Procedure for Preparation of 6-[8-(1, 3-benzothiazol-2-ylcarbamoyl)-3, 4-dihydro-1H-isoquinolin-2-yl]-3-[3-[3-[6-[2-[[3-(2, 6-dioxo-3-piperidyl)-1-methyl-indazol-6-yl]amino]-2-oxo-ethyl]-3-pyridyl]propoxy]-2-methyl-phenyl]pyridine-2-carboxylic acid

To a solution of tert-butyl 6-[8-(1, 3-benzothiazol-2-ylcarbamoyl)-3, 4-dihydro-1H-isoquinolin-2-yl]-3-[3-[3-[6-[2-[[3-(2, 6-dioxo-3-piperidyl)-1-methyl-indazol-6-yl]amino]-2-oxo-ethyl]-3-pyridyl]propoxy]-2-methyl-phenyl]pyridine-2-carboxylate (80 mg, 50.92 μmol, 64.3% purity, 1 equiv.) in DCM (0.5 mL) and TFA (0.5 mL) was stirred at 25° C. for 16 hours. The reaction mixture was filtered and concentrated under reduced pressure to give a residue. The residue was purified by prep-HPLC. The compound 6-[8-(1, 3-benzothiazol-2-ylcarbamoyl)-3, 4-dihydro-1H-isoquinolin-2-yl]-3-[3-[3-[6-[2-[[3-(2, 6-dioxo-3-piperidyl)-1-methyl-indazol-6-yl]amino]-2-oxo-ethyl]-3-pyridyl]propoxy]-2-methyl-phenyl]pyridine-2-carboxylic acid (21.3 mg, 20.9 μmol, 41.2% yield, 93.7% purity) was obtained as a yellow solid.

MS (ESI) m/z: 954.3 [M+H]⁺.

¹H NMR (400 MHz, DMSO-d₆) δ=10.86 (s, 1H), 10.43 (s, 1H), 8.39 (d, J=2.0 Hz, 1H), 8.07 (s, 1H), 8.02 (d, J=8.0 Hz, 1H), 7.78 (d, J=7.6 Hz, 1H), 7.67-7.60 (m, 3H), 7.49-7.42 (m, 3H), 7.39-7.32 (m, 3H), 7.14-7.05 (m, 2H), 6.95 (d, J=8.8 Hz, 1H), 6.86 (d, J=8.0 Hz, 1H), 6.64 (d, J=7.2 Hz, 1H), 4.98 (s, 2H), 4.31 (dd, J=5.2, 9.6 Hz, 1H), 4.00-3.96 (m, 2H), 3.94-3.90 (m, 2H), 3.89 (s, 3H), 3.85 (s, 2H), 3.02 (t, J=5.6 Hz, 2H), 2.78 (t, J=7.2 Hz, 2H), 2.67-2.62 (m, 2H), 2.37-2.29 (m, 1H), 2.21-2.13 (m, 1H), 2.10-2.02 (m, 2H), 1.92 (s, 3H)

### Example 81. Preparation of Compound 289b

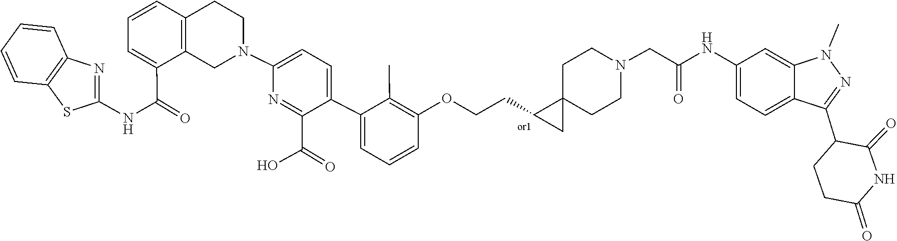 [SMILES: Cc1c(OCC[C@H]2CC23CCN(CC(=O)Nc2ccc4c(C5CCC(=O)NC5=O)nn(C)c4c2)CC3)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(2-((1R)-6-(2-((3-(2,6-dioxopiperidin-3-yl)-1-methyl-1H-indazol-6-yl)amino)-2-oxoethyl)-6-azaspiro[2.5]octan-1-yl)ethoxy)-2-methylphenyl)picolinic acid

### [rel-6-(8-(benzo[d]thiazol-2-ylcarbamoyl)-3,4-dihydroisoquinolin-2(1H)-yl)-3-(3-(2-((1R)-6-(2-((3-(2,6-dioxopiperidin-3-yl)-1-methyl-1H-indazol-6-yl)amino)-2-oxoethyl)-6-azaspiro[2.5]octan-1-yl)ethoxy)-2-methylphenyl)picolinic acid]

### Step A. Procedure for Preparation of 6-(tert-butyl) 1-methyl 6-azaspiro[2.5]octane-1,6-dicarboxylate

A mixture of 6-(tert-butoxycarbonyl)-6-azaspiro[2.5]octane-1-carboxylic acid (5 g, 19.58 mmol, 1 equiv.), Cs₂CO₃ (7.66 g, 23.50 mmol, 1.2 equiv.) in DMF (50 mL), then added Mel (3.34 g, 23.50 mmol, 1.46 mL, 1.2 equiv.) to the mixture, the mixture was stirred at 25° C. for 2 hours, The mixture was diluted with water (10 mL) and extracted with EtOAc 15 mL (5 mL×3). The combined organic layers were washed with brine (5 mL×2), dried over Na₂SO₄, filtered and concentrated under reduced pressure to give the compound 6-(tert-butyl) 1-methyl 6-azaspiro[2.5]octane-1,6-dicarboxylate (5 g, crude) as a colorless oil.

¹H NMR (400 MHz, DMSO-d₆) δ=3.60 (s, 3H), 3.41-3.26 (m, 4H), 3.17-3.09 (m, 1H), 1.72-1.43 (m, 4H), 1.39 (s, 9H), 0.99 (d, J=6.8 Hz, 2H)

### Step B. Procedure for Preparation of tert-butyl 1-(hydroxymethyl)-6-azaspiro[2.5]octane-6-carboxylate

To a solution of 6-(tert-butyl) 1-methyl 6-azaspiro[2.5]octane-1,6-dicarboxylate (5 g, crude) in THE (100 mL) was added LAH (1.69 g, 44.55 mmol, 1.2 equiv.) at 0° C. Then the mixture was stirred at 20° C. for 2 hours. The reaction mixture was quenched by addition Na₂SO₄·10H₂O (1 g) under 0° C. and N₂ atmosphere, and then filtered and concentrated under reduced pressure to give a residue. The residue was used for next step without other purification. The compound tert-butyl 1-(hydroxymethyl)-6-azaspiro[2.5]octane-6-carboxylate (8.4 g, 34.8 mmol, 93.8% yield) was obtained as a yellow oil.

¹H NMR (400 MHz, DMSO-d₆) δ=5.75 (s, 1H), 4.40 (t, J=5.2 Hz, 1H), 3.56-3.43 (m, 2H), 3.38-3.33 (m, 1H), 3.30-3.21 (m, 3H), 1.39 (s, 9H), 1.36-1.25 (m, 2H), 1.22-1.15 (m, 1H), 0.87-0.77 (m, 1H), 0.43 (dd, J=4.4, 8.8 Hz, 1H), 0.14 (t, J=4.8 Hz, 1H)

### Step C. Procedure for Preparation of tert-butyl 1-formyl-6-azaspiro[2.5]octane-6-carboxylate

A mixture of DMSO (8.29 g, 106.08 mmol, 8.29 mL, 4 equiv.) in DCM (65 mL) was added dropwise to a solution of (COCl)₂ (6.73 g, 53.04 mmol, 4.64 mL, 2 equiv.) in DCM (10 mL) at −70° C. under N₂ atmosphere, The mixture was stirred at −70° C. for 1 hour, tert-butyl 1-(hydroxymethyl)-6-azaspiro[2.5]octane-6-carboxylate (6.4 g, 26.52 mmol, 1 equiv.) in DCM (10 mL) and was added dropwise at −70° C. The solution was stirred for 1 hour at −70° C. Then TEA (16.10 g, 159.12 mmol, 22.15 mL, 6 equiv.) was added into the solution. The solution was stirred at −70° C. for 0.5 hour under N₂ atmosphere. The mixture was diluted with H₂O (10 mL) and extracted with DCM 15 mL (5 mL×3). The combined organic layers were filtered and concentrated under reduced pressure to give a residue. The residue was purified by flash silica gel chromatography (Eluent of 0˜ 30% ethyl acetate/petroleum ether) to give tert-butyl 1-formyl-6-azaspiro[2.5]octane-6-carboxylate (4.7 g, 19.6 mmol, 74.1% yield) as a yellow oil.

### Step D. Procedure for Preparation of tert-butyl (E)-1-(2-methoxyvinyl)-6-azaspiro[2.5]octane-6-carboxylate

A mixture of methoxymethyl(triphenyl)phosphonium; bromide (9.71 g, 25.07 mmol, 2 equiv.) in THE (25 mL) was added LiHMDS (1 M, 25.07 mL, 2 equiv.) at 0° C. for 1 hour under N₂ atmosphere, then tert-butyl 1-formyl-6-azaspiro[2.5]octane-6-carboxylate (3 g, 12.54 mmol, 1 equiv.) in THE (5 mL) was added dropwise at 0° C., the resulting mixture was stirred at 20° C. for 1 hour under N₂ atmosphere. The reaction mixture was quenched by addition sat. NH₄Cl (5 mL) at 0° C., and then diluted with EtOAc (1 mL) and extracted with 3 mL (1 mL×3). The combined organic layers were washed with H₂O (3 mL), dried over Na₂SO₄, filtered, and concentrated under reduced pressure to give a residue. The residue was purified by flash silica gel chromatography (Eluent of 0˜8% ethyl acetate/petroleum ether) to give tert-butyl (E)-1-(2-methoxyvinyl)-6-azaspiro[2.5]octane-6-carboxylate (4 g, crude) as a yellow oil.

¹H NMR (400 MHz, CDCl₃) δ=5.97 (dd, J=0.8, 6.4 Hz, 1H), 4.07 (dd, J=6.4, 9.2 Hz, 1H), 3.62 (s, 3H), 1.46-1.45 (m, 12H), 1.40-1.32 (m, 4H), 1.28-1.15 (m, 1H), 0.75 (dd, J=4.4, 8.4 Hz, 1H), 0.35-0.29 (m, 2H)

### Step E. Procedure for Preparation of tert-butyl 1-(2-oxoethyl)-6-azaspiro[2.5]octane-6-carboxylate

A mixture of tert-butyl (E)-1-(2-methoxyvinyl)-6-azaspiro[2.5]octane-6-carboxylate (3 g, 11.22 mmol, 1 equiv.), TFA (3.84 g, 33.66 mmol, 2.49 mL, 3 equiv.), in H₂O (30 mL) and CH₃CN (120 mL) was stirred at 25° C. for 16 hours. The reaction mixture was quenched by addition NaHCO₃ (5 mL), and then diluted with H₂O (20 mL) and extracted with DCM 90 mL (30 mL×3). Then the combined organic layers were filtered and concentrated. The residue was used for next step without other purification. The compound tert-butyl 1-(2-oxoethyl)-6-azaspiro[2.5]octane-6-carboxylate (3 g, crude) was obtained as a yellow oil.

### Step F. Procedure for Preparation of tert-butyl 1-(2-hydroxyethyl)-6-azaspiro[2.5]octane-6-carboxylate

To a solution of tert-butyl 1-(2-oxoethyl)-6-azaspiro[2.5]octane-6-carboxylate (3 g, 11.84 mmol, 1 equiv.) in MeOH (30 mL) was added NaBH₄ (537.58 mg, 14.21 mmol, 1.2 equiv.) at 0° C., The mixture was stirred at 20° C. for 1 hour. The reaction mixture was quenched by addition sat. NH₄Cl (5 mL) at 0° C., and then diluted with ethyl acetate (1 mL) and extracted with 3 mL (1 mL×3). The combined organic layers were washed with H₂O 3 mL, dried over Na₂SO₄, filtered, and concentrated under reduced pressure to give a residue. The residue was purified by flash silica gel chromatography (Eluent of 0˜30% ethyl acetate/petroleum ether) to give tert-butyl 1-(2-hydroxyethyl)-6-azaspiro[2.5]octane-6-carboxylate (1.8 g, 7.0 mmol, 59.5% yield) as a colorless oil.

¹H NMR (400 MHz, CDCl₃) δ=3.75-3.69 (m, 2H), 3.67-3.56 (m, 2H), 3.26-3.20 (m, 2H), 1.82-1.74 (m, 1H), 1.64-1.58 (m, 2H), 1.46 (s, 9H), 1.44-1.37 (m, 1H), 1.36-1.27 (m, 1H), 1.17-1.09 (m, 1H), 0.68-0.61 (m, 1H), 0.53-0.50 (m, 1H), 0.07 (t, J=4.8 Hz, 1H)

### Step G. Procedure for Preparation of tert-butyl 1-(2-(3-bromo-2-methylphenoxy)ethyl)-6-azaspiro[2.5]octane-6-carboxylate

A mixture of tert-butyl 1-(2-hydroxyethyl)-6-azaspiro[2.5]octane-6-carboxylate (1 g, 3.92 mmol, 1.2 equiv.), 3-bromo-2-methyl-phenol (610.38 mg, 3.26 mmol, 1 equiv.), 2-(tributyl-λ⁵-phosphanylidene)acetonitrile (1.18 g, 4.90 mmol, 1.5 equiv.), in toluene (10 mL) was stirred at 120° C. for 16 hours. The reaction mixture was concentrated under reduced pressure to give a residue. The residue was purified by flash silica gel chromatography (Eluent of 0˜ 20% ethyl acetate/petroleum ether) to give tert-butyl 1-(2-(3-bromo-2-methylphenoxy)ethyl)-6-azaspiro[2.5]octane-6-carboxylate (1.3 g, 3.1 mmol, 93.9% yield) as a yellow oil.

MS (ESI) m/z: 368.1[M+H]⁺.

### Step H. Procedure for Preparation of 1-(2-(3-bromo-2-methylphenoxy)ethyl)-6-azaspiro[2.5]octane

A mixture of tert-butyl 1-(2-(3-bromo-2-methylphenoxy)ethyl)-6-azaspiro[2.5]octane-6-carboxylate (1.3 g, 3.06 mmol, 1 equiv.), in TFA (3 mL) and DCM (9 mL) was stirred at 25° C. for 2 hours. The combined organic layers were filtered and concentrated under reduced pressure to give a residue. The residue was used for next step without other purification. The compound 1-(2-(3-bromo-2-methylphenoxy)ethyl)-6-azaspiro[2.5]octane (1.3 g, crude, TFA) was obtained as a white solid.

MS (ESI) m/z: 323.9[M+H]⁺.

### Step I. Procedure for Preparation of ethyl 2-(1-(2-(3-bromo-2-methylphenoxy)ethyl)-6-azaspiro[2.5]octan-6-yl)acetate

A mixture of 1-(2-(3-bromo-2-methylphenoxy)ethyl)-6-azaspiro[2.5]octane (1.3 g, 2.97 mmol, 1 equiv., TFA), ethyl 2-bromoacetate (544.89 mg, 3.26 mmol, 360.85 μL, 1.1 equiv.), TEA (1.50 g, 14.83 mmol, 2.06 mL, 5 equiv.), in CH₃CN (10 mL) was stirred at 25° C. for 1 hour. The combined organic layers were filtered and concentrated under reduced pressure to give a residue The residue was purified by flash silica gel chromatography (Eluent of 0˜50% ethyl acetate/petroleum ether) to give ethyl 2-(1-(2-(3-bromo-2-methylphenoxy)ethyl)-6-azaspiro[2.5]octan-6-yl)acetate (1 g, 2.4 mmol, 80.6% yield, 98.0% purity) as a colorless oil.

MS (ESI) m/z: 410.0 [M+H]⁺.

### Step J. Procedure for Preparation of ethyl (R)-2-(1-(2-(3-bromo-2-methylphenoxy)ethyl)-6-azaspiro[2.5]octan-6-yl)acetate

The ethyl 2-(1-(2-(3-bromo-2-methylphenoxy)ethyl)-6-azaspiro[2.5]octan-6-yl)acetate residue was purified by prep-HPLC to give ethyl (S)-2-(1-(2-(3-bromo-2-methylphenoxy)ethyl)-6-azaspiro[2.5]octan-6-yl)acetate (450 mg, 1.10 mmol, 45.00% yield) as a colorless oil and ethyl (R)-2-(1-(2-(3-bromo-2-methylphenoxy)ethyl)-6-azaspiro[2.5]octan-6-yl)acetate (390 mg, 950.4 μmol, 39.00% yield) as a colorless oil.

### Step K. Procedure for Preparation of tert-butyl (R)-6-(8-(benzo[d]thiazol-2-ylcarbamoyl)-3,4-dihydroisoquinolin-2(1H)-yl)-3-(3-(2-(6-(2-ethoxy-2-oxoethyl)-6-azaspiro[2.5]octan-1-yl)ethoxy)-2-methylphenyl)picolinate

A mixture of ethyl (R)-2-(1-(2-(3-bromo-2-methylphenoxy)ethyl)-6-azaspiro[2.5]octan-6-yl)acetate (100.00 mg, 243.70 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (179.13 mg, 292.44 μmol, 1.2 equiv.), [2-(2-aminophenyl)phenyl]palladium(1+); bis(1-adamantyl)-butyl-phosphane; methanesulfonate (35.50 mg, 48.74 μmol, 0.2 equiv.), K₂CO₃ (1.5 M, 487.40 μL, 3 equiv.) in dioxane (1 mL) was degassed and purged with N₂ three times, and then the mixture was stirred at 100° C. for 1 hour under N₂ atmosphere under MW. The combined organic layers were filtered and concentrated under reduced pressure to give a residue. The residue was purified by flash silica gel chromatography (Eluent of 0˜60% ethyl acetate/petroleum ether) to give tert-butyl (R)-6-(8-(benzo[d]thiazol-2-ylcarbamoyl)-3,4-dihydroisoquinolin-2(1H)-yl)-3-(3-(2-(6-(2-ethoxy-2-oxoethyl)-6-azaspiro[2.5]octan-1-yl)ethoxy)-2-methylphenyl)picolinate (130 mg, 159.3 μmol, 65.4% yield) as a yellow oil.

MS (ESI) m/z: 816.3 [M+H]⁺.

### Step L. Procedure for Preparation of (R)-2-(1-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethyl)-6-azaspiro[2.5]octan-6-yl)acetic acid

A mixture of tert-butyl (R)-6-(8-(benzo[d]thiazol-2-ylcarbamoyl)-3,4-dihydroisoquinolin-2(1H)-yl)-3-(3-(2-(6-(2-ethoxy-2-oxoethyl)-6-azaspiro[2.5]octan-1-yl)ethoxy)-2-methylphenyl)picolinate (130 mg, 159.31 μmol, 1 equiv.), LiOH·H₂O (20.06 mg, 477.93 μmol, 3 equiv.), in THF (0.8 mL) and H₂O (0.4 mL) was stirred at 25° C. for 2 hours. The reaction mixture was diluted with H₂O (5 mL) and concentrated as a turbid liquid. Then the pH of the turbid liquid was adjusted to 3 with 1 N HCl (5 mL), the residue was extracted with DCM/MeOH(20:1), The combined organic layers were filtered and concentrated. The residue was used for next step without other purification. The compound (R)-2-(1-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethyl)-6-azaspiro[2.5]octan-6-yl)acetic acid (100 mg, 126.9 μmol, 79.7% yield) was obtained as a yellow solid.

MS (ESI) m/z: 788.4 [M+H]⁺.

### Step M. Procedure for Preparation of tert-butyl 6-(8-(benzo[d]thiazol-2-ylcarbamoyl)-3,4-dihydroisoquinolin-2(1H)-yl)-3-(3-(2-((1R)-6-(2-((3-(2,6-dioxopiperidin-3-yl)-1-methyl-11H-indazol-6-yl)amino)-2-oxoethyl)-6-azaspiro[2.5]octan-1-yl)ethoxy)-2-methylphenyl)picolinate

A mixture of (R)-2-(1-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethyl)-6-azaspiro[2.5]octan-6-yl)acetic acid (90 mg, 114.22 μmol, 1 equiv.), 3-(6-amino-1-methyl-indazol-3-yl)piperidine-2,6-dione (35.40 mg, 137.06 μmol, 1.2 equiv.), EDCI (32.84 mg, 171.33 μmol, 1.5 equiv.), in pyridine (0.5 mL) was stirred at 25° C. for 2 hour The mixture was diluted with H₂O mL (10 mL×3) and extracted with ethyl acetate 24 mL (8 mL×3). The combined organic layers were filtered and concentrated under reduced pressure to give a residue. The residue was used for next step without other purification. The compound tert-butyl 6-(8-(benzo[d]thiazol-2-ylcarbamoyl)-3,4-dihydroisoquinolin-2(1H)-yl)-3-(3-(2-((1R)-6-(2-((3-(2,6-dioxopiperidin-3-yl)-1-methyl-1H-indazol-6-yl)amino)-2-oxoethyl)-6-azaspiro[2.5]octan-1-yl)ethoxy)-2-methylphenyl)picolinate (90 mg, crude) was obtained as a yellow solid.

MS (ESI) m/z: 1028.5 [M+H]⁺.

### Step N. Procedure for Preparation of 6-(8-(benzo[d]thiazol-2-ylcarbamoyl)-3,4-dihydroisoquinolin-2(1H)-yl)-3-(3-(2-((1R)-6-(2-((3-(2,6-dioxopiperidin-3-yl)-1-methyl-11H-indazol-6-yl)amino)-2-oxoethyl)-6-azaspiro[2.5]octan-1-yl)ethoxy)-2-methylphenyl)picolinic acid [rel-6-(8-(benzo[d]thiazol-2-ylcarbamoyl)-3,4-dihydroisoquinolin-2(1H)-yl)-3-(3-(2-((1R)-6-(2-((3-(2,6-dioxopiperidin-3-yl)-1-methyl-1H-indazol-6-yl)amino)-2-oxoethyl)-6-azaspiro[2.5]octan-1-yl)ethoxy)-2-methylphenyl)picolinic acid]

A mixture of tert-butyl 6-(8-(benzo[d]thiazol-2-ylcarbamoyl)-3,4-dihydroisoquinolin-2(1H)-yl)-3-(3-(2-((1R)-6-(2-((3-(2,6-dioxopiperidin-3-yl)-1-methyl-1H-indazol-6-yl)amino)-2-oxoethyl)-6-azaspiro[2.5]octan-1-yl)ethoxy)-2-methylphenyl)picolinate (90 mg, 87.53 μmol, 1 equiv.) in TFA (0.8 mL) and DCM (0.8 mL) was stirred at 25° C. for 2 hours, The combined organic layers were filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(2-((1R)-6-(2-((3-(2,6-dioxopiperidin-3-yl)-1-methyl-1H-indazol-6-yl)amino)-2-oxoethyl)-6-azaspiro[2.5]octan-1-yl)ethoxy)-2-methylphenyl)picolinic acid [rel-6-(8-(benzo[d]thiazol-2-ylcarbamoyl)-3,4-dihydroisoquinolin-2(1H)-yl)-3-(3-(2-((1R)-6-(2-((3-(2,6-dioxopiperidin-3-yl)-1-methyl-1H-indazol-6-yl)amino)-2-oxoethyl)-6-azaspiro[2.5]octan-1-yl)ethoxy)-2-methylphenyl)picolinic acid] (98.7% purity) as a white solid.

MS (ESI) m/z: 972.3 [M+H]⁺.

¹H NMR (400 MHz, DMSO-d₆) δ=12.98-12.77 (m, 1H), 10.88 (s, 1H), 10.44-9.44 (m, 1H), 8.07-8.01 (m, 2H), 7.78 (d, J=8.0 Hz, 1H), 7.69-7.59 (m, 2H), 7.49-7.32 (m, 5H), 7.23-7.15 (m, 1H), 7.10 (t, J=7.6 Hz, 1H), 6.99-6.88 (m, 2H), 6.63 (d, J=7.6 Hz, 1H), 4.97 (s, 2H), 4.33 (dd, J=5.2, 9.6 Hz, 1H), 4.13-4.02 (m, 2H), 3.94-3.89 (m, 5H), 3.47-3.35 (m, 6H), 3.02 (t, J=5.2 Hz, 2H), 2.69-2.53 (m, 4H), 2.37-2.31 (m, 1H), 2.21-2.13 (m, 1H), 1.92 (s, 3H), 1.76-1.57 (m, 2H), 1.47-1.01 (m, 2H), 0.92-0.68 (m, 1H), 0.61-0.35 (m, 1H), 0.23-0.00 (m, 1H)

### Example 82. Preparation or Compound 290

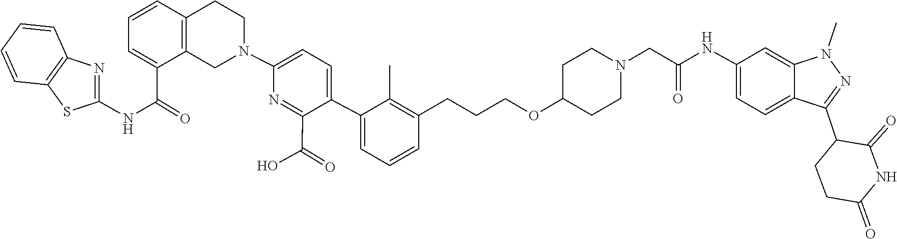 [SMILES: Cc1c(CCCOC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[[1-[2-[[3-(2,6-dioxo-3-piperidyl)-1-methyl-indazol-6-yl]amino]-2-oxo-ethyl]-4-piperidyl]oxy]propyl]-2-methyl-phenyl]pyridine-2-carboxylic acid

### Step A. Procedure for Preparation of tert-butyl 4-prop-2-ynoxypiperidine-1-carboxylate

A solution of tert-butyl 4-hydroxypiperidine-1-carboxylate (10 g, 49.69 mmol, 1.0 equiv.) in anhydrous THF (50 mL) was cooled to 0° C. Subsequently, NaH (2.38 g, 59.62 mmol, 60% purity, 1.2 equiv.) was added under N₂ atmosphere. The reaction mixture was stirred at 0° C. for 0.5 hours. Then, 3-bromoprop-1-yne (7.39 g, 49.69 mmol, 5.35 mL, 1.0 equiv.) was added. The resulting reaction mixture was stirred at 25° C. for 15.5 hours. Once completed, the reaction mixture was quenched with water (1 mL), then diluted with ethyl acetate (100 mL). The organic layers were washed with brine (20 mL), dried over anhydrous sodium sulfate, and filtered. The filtrate was concentrated in vacuo. The residue was purified by flash silica gel chromatography (Eluent of 0˜10% ethyl acetate/petroleum ether) to give tert-butyl 4-prop-2-ynoxypiperidine-1-carboxylate (8.1 g, 29.9 mmol, 60.1% yield, 88% purity) as a yellow oil.

¹H NMR (400 MHz, DMSO-d₆) δ=4.17 (d, J=2.4 Hz, 2H), 3.69-3.56 (m, 3H), 3.41-3.36 (m, 1H), 3.03 (t, J=9.6 Hz, 2H), 1.83-1.75 (m, 2H), 1.45-1.37 (m, 9H), 1.36-1.30 (m, 2H)

### Step B. Procedure for Preparation of tert-butyl 4-[3-(3-bromo-2-methyl-phenyl) prop-2-ynoxy]piperidine-1-carboxylate

A mixture of tert-butyl 4-prop-2-ynoxypiperidine-1-carboxylate (2.1 g, 8.78 mmol, 1.0 equiv.), 1-bromo-3-iodo-2-methyl-benzene (2.61 g, 8.78 mmol, 1.0 equiv.), TEA (2.66 g, 26.33 mmol, 3.66 mL, 3.0 equiv.), CuI (334.25 mg, 1.76 mmol, 0.2 equiv.), and Pd(PPh₃)₂Cl₂ (615.93 mg, 877.52 μmol, 0.1 equiv.) in DMF (30 mL) was degassed and purged with N₂ for three times, and then the mixture was stirred at 60° C. for 2 hours under N₂ atmosphere. The reaction mixture was partitioned between ethyl acetate (150 mL) and H₂O (150 mL). The organic phase was separated, washed with NaCl (aq.) (100 mL×3), dried over Na₂SO₄, filtered, and concentrated under reduced pressure to give a residue. The residue was purified by flash silica gel chromatography (Eluent of 0˜8% ethyl acetate/petroleum ether) to give tert-butyl 4-[3-(3-bromo-2-methyl-phenyl) prop-2-ynoxy]piperidine-1-carboxylate (2.6 g, 6.3 mmol, 71.5% yield, 99% purity) as a yellow solid.

¹H NMR (400 MHz, DMSO-d₆) δ=7.63 (dd, J=0.8, 8.0 Hz, 1H), 7.49-7.43 (m, 1H), 7.15 (t, J=8.0 Hz, 1H), 4.49 (s, 2H), 3.78-3.69 (m, 1H), 3.68-3.61 (m, 2H), 3.05 (s, 2H), 2.48 (s, 3H), 1.94-1.78 (m, 2H), 1.40 (s, 11H).

### Step C. Procedure for Preparation of tert-butyl 4-[3-(3-bromo-2-methyl-phenyl)propoxy]piperidine-1-carboxylate

To a solution of tert-butyl 4-[3-(3-bromo-2-methyl-phenyl) prop-2-ynoxy]piperidine-1-carboxylate (1.5 g, 3.67 mmol, 1.0 equiv.) in EtOAc (10 mL) was added Rh/Al₂O₃ (75.61 mg, 734.70 μmol, 0.2 equiv.). The mixture was stirred at 25° C. for 6 hours. Filtration of the reaction mixture then gives the crude product tert-butyl 4-[3-(3-bromo-2-methyl-phenyl)propoxy]piperidine-1-carboxylate (1.4 g, 3.5 mmol, 95.7% yield) as yellow oil.

MS (ESI) m/z: 356.1 [M-56+H]⁺

### Step D. Procedure for Preparation of 4-[3-(3-bromo-2-methyl-phenyl)propoxy] piperidine

To a solution of tert-butyl 4-[3-(3-bromo-2-methyl-phenyl)propoxy]piperidine-1-carboxylate (1.4 g, 3.40 mmol, 1.0 equiv.) in HCl/EtOAc (50 mL) and EtOAc (10 mL). The mixture was stirred at 25° C. for 1 hour. The reaction mixture was concentrated under reduced pressure to remove HCl/EtOAc (50 mL) and EtOAc (10 mL) to give the crude product 4-[3-(3-bromo-2-methyl-phenyl)propoxy] piperidine (1.01 g, 3.2 mmol, 95.3% yield) as yellow oil.

MS (ESI) m/z: 312.2 [M+H]⁺

### Step E. Procedure for Preparation of ethyl 2-[4-[3-(3-bromo-2-methyl-phenyl)propoxy]-1-piperidyl]acetate

To a solution of ethyl 2-bromoacetate (420.10 mg, 2.52 mmol, 278.40 μL, 1.1 equiv.) and 4-[3-(3-bromo-2-methyl-phenyl)propoxy]piperidine (800 mg, 2.29 mmol, 1.0 equiv., HCl) and KI (380.84 mg, 2.29 mmol, 1.0 equiv.) in CH₃CN (5 mL) was added TEA (696.44 mg, 6.88 mmol, 957.97 μL, 3.0 equiv.). The mixture was stirred at 25° C. for 1 hour. The reaction mixture was concentrated under reduced pressure. The residue was diluted with H₂O (50 mL) and extracted with ethyl acetate (50 mL×3). The combined organic layers were washed with NaCl (aq.) (50 mL×3), dried over Na₂SO₄, filtered, and concentrated under reduced pressure to give a residue. The residue was purified by flash silica gel chromatography (Eluent of 30˜50% ethyl acetate/petroleum ether) to give ethyl 2-[4-[3-(3-bromo-2-methyl-phenyl)propoxy]-1-piperidyl]acetate (785 mg, 1.54 mmol, 67.00% yield, 78% purity) as a yellow oil.

MS (ESI) m/z: 399.2 [M+H]⁺

### Step F. Procedure for Preparation of tert-butyl 6-[8-(1,3-benzothiazol-2-ylcarbamoyl)-3,4-dihydro-1H-isoquinolin-2-yl]-3-[3-[3-[[1-(2-ethoxy-2-oxo-ethyl)-4-piperidyl]oxy]propyl]-2-methyl-phenyl]pyridine-2-carboxylate

A mixture of ethyl 2-[4-[3-(3-bromo-2-methyl-phenyl)propoxy]-1-piperidyl]acetate (400 mg, 1.00 mmol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (615.11 mg, 1.00 mmol, 1.0 equiv.), [2-(2-aminophenyl)phenyl]palladium(1+); bis(1-adamantyl)-butyl-phosphane; methanesulfonate (73.13 mg, 100.42 μmol, 0.1 equiv.), K₂CO₃ (416.35 mg, 3.01 mmol, 3.0 equiv.) and in dioxane (8 mL) and H₂O (1 mL) was degassed and purged with N₂ three times, and then the mixture was stirred at 100° C. for 1 hour under N₂ atmosphere under microwave. The reaction mixture was concentrated under reduced pressure to remove dioxane (8 mL). The residue was diluted with H₂O (50 mL) and extracted with ethyl acetate (50 mL×3). The combined organic layers were dried over Na₂SO₄, filtered, and concentrated under reduced pressure to give a residue. The residue was purified by flash silica gel chromatography (Eluent of 50˜80% ethyl acetate/petroleum ether) to give tert-butyl 6-[8-(1,3-benzothiazol-2-ylcarbamoyl)-3,4-dihydro-1H-isoquinolin-2-yl]-3-[3-[3-[[1-(2-ethoxy-2-oxo-ethyl)-4-piperidyl]oxy]propyl]-2-methyl-phenyl]pyridine-2-carboxylate (353 mg, 424.8 μmol, 42.3% yield, 96.8% purity) as yellow solid.

MS (ESI) m/z: 804.6 [M+H]⁺

### Step G. Procedure for Preparation of 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenyl]propoxy]-1-piperidyl]acetic acid

To a solution of tert-butyl 6-[8-(1,3-benzothiazol-2-ylcarbamoyl)-3,4-dihydro-1H-isoquinolin-2-yl]-3-[3-[3-[[1-(2-ethoxy-2-oxo-ethyl)-4-piperidyl]oxy]propyl]-2-methyl-phenyl]pyridine-2-carboxylate (333 mg, 414.18 μmol, 1.0 equiv.) in THE (10 mL) was added LiOH·H₂O (69.52 mg, 1.66 mmol, 4.0 equiv.) and H₂O (10 mL). The mixture was stirred at 25° C. for 2 hours. The reaction mixture was concentrated under reduced pressure to remove THF. Then H₂O (30 mL) was added to the mixture, and its pH was adjusted to 3-4 with HCl (1 N). The mixture was filtered to give 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenyl]propoxy]-1-piperidyl]acetic acid (253 mg, 326.0 mol, 78.7% yield) as a yellow solid.

MS (ESI) m/z: 776.5 [M+H]⁺

### Step H. Procedure for Preparation of tert-butyl 6-[8-(1,3-benzothiazol-2-ylcarbamoyl)-3,4-dihydro-1H-isoquinolin-2-yl]-3-[3-[3-[[1-[2-[[3-(2,6-dioxo-3-piperidyl)-1-methyl-indazol-6-yl]amino]-2-oxo-ethyl]-4-piperidyl]oxy]propyl]-2-methyl-phenyl]pyridine-2-carboxylate

To a solution of 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenyl]propoxy]-1-piperidyl]acetic acid (200 mg, 257.75 μmol, 1.0 equiv.) in pyridine (8 mL) was added EDCI (74.12 mg, 386.62 μmol, 1.5 equiv.) and 3-(6-amino-1-methyl-indazol-3-yl)piperidine-2,6-dione (66.57 mg, 257.75 μmol, 1.0 equiv.) The mixture was stirred at 25° C. for 16 hours. The reaction mixture was concentrated under reduced pressure to give a residue. Tert-butyl 6-[8-(1,3-benzothiazol-2-ylcarbamoyl)-3,4-dihydro-1H-isoquinolin-2-yl]-3-[3-[3-[[1-[2-[[3-(2,6-dioxo-3-piperidyl)-1-methyl-indazol-6-yl]amino]-2-oxo-ethyl]-4-piperidyl]oxy]propyl]-2-methyl-phenyl]pyridine-2-carboxylate (215 mg, crude) was obtained as a yellow solid.

MS (ESI) m/z: 1016.5 [M+H]⁺

### Step L Procedure for preparation of 6-[8-(1,3-benzothiazol-2-ylcarbamoyl)-3,4-dihydro-1H-isoquinolin-2-yl]-3-[3-[3-[[1-[2-[[3-(2,6-dioxo-3-piperidyl)-1-methyl-indazol-6-yl]amino]-2-oxo-ethyl]-4-piperidyl]oxy]propyl]-2-methyl-phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[3-[[1-[2-[[3-(2,6-dioxo-3-piperidyl)-1-methyl-indazol-6-yl]amino]-2-oxo-ethyl]-4-piperidyl]oxy]propyl]-2-methyl-phenyl]pyridine-2-carboxylate (150 mg, 147.61 μmol, 1 equiv.) in TFA (10 mL) and DCM (10 mL) was stirred at 30° C. for 2 hours. The reaction mixture was concentrated under reduced pressure to get a residue. The residue was purified by prep-HPLC to give 6-[8-(1,3-benzothiazol-2-ylcarbamoyl)-3,4-dihydro-1H-isoquinolin-2-yl]-3-[3-[3-[[1-[2-[[3-(2,6-dioxo-3-piperidyl)-1-methyl-indazol-6-yl]amino]-2-oxo-ethyl]-4-piperidyl]oxy]propyl]-2-methyl-phenyl]pyridine-2-carboxylic acid (90.0 mg, 93.3 μmol, 63.2% yield, 99.5% purity) as a yellow solid.

MS (ESI) m/z: 960.5 [M+H]⁺

¹H NMR (400 MHz, DMSO-d₆) δ=13.07-12.64 (m, 1H), 10.89 (s, 1H), 9.88 (s, 1H), 8.14 (s, 1H), 8.09-7.96 (m, 2H), 7.79 (d, J=8.0 Hz, 1H), 7.63 (d, J=8.4 Hz, 2H), 7.52-7.43 (m, 3H), 7.37 (d, J=7.6, 12.0 Hz, 2H), 7.24 (d, J=8.4 Hz, 1H), 7.14-7.04 (m, 2H), 6.98 (d, J=8.8 Hz, 1H), 6.87 (d, J=6.8 Hz, 1H), 4.99 (s, 2H), 4.33 (dd, J=5.2, 9.6 Hz, 1H), 3.92 (s, 5H), 3.44 (t, J=6.0 Hz, 2H), 3.16 (s, 2H), 3.03 (t, J=5.6 Hz, 2H), 2.83-2.75 (m, 2H), 2.70-2.61 (m, 4H), 2.38-2.29 (m, 3H), 2.22-2.14 (m, 1H), 2.01 (s, 3H), 1.88 (d, J=9.6 Hz, 2H), 1.80-1.70 (m, 2H), 1.64-1.53 (m, 2H)

### Example 83. Preparation of Compound 291

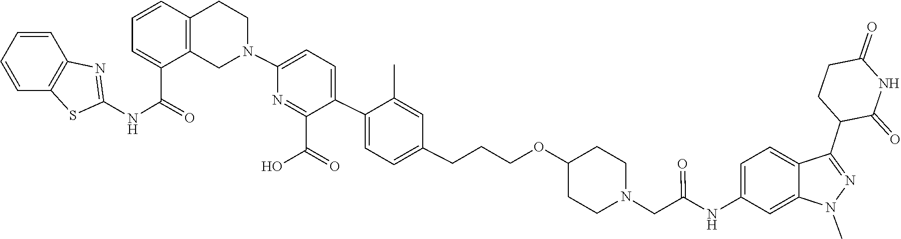 [SMILES: Cc1cc(CCCOC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[4-[3-[[1-[2-[[3-(2,6-dioxo-3-piperidyl)-1-methyl-indazol-6-yl]amino]-2-oxo-ethyl]-4-piperidyl]oxy]propyl]-2-methyl-phenyl]pyridine-2-carboxylic acid

### Step A. Procedure for Preparation of tert-butyl 4-prop-2-ynoxypiperidine-1-carboxylate

To a solution of tert-butyl 4-hydroxypiperidine-1-carboxylate (2 g, 9.94 mmol, 1 equiv.) in THE (20 mL) was cooled to 0° C. NaH (476.94 mg, 11.92 mmol, 60% purity, 1.2 equiv.) was added into the mixture at 0° C. The reaction mixture was stirred at 0° C. for 0.5 hour. Then 3-bromoprop-1-yne (1.18 g, 9.94 mmol, 856.62 μL, 1 equiv.) was added into the mixture. The reaction mixture was stirred at 25° C. for 12 hours. Once completed, the reaction mixture was quenched with water (20 mL), then extracted with ethyl acetate (20 mL×3). The organic layers were washed with brine (20 mL), dried over anhydrous sodium sulfate, filtered, and concentrated in vacuo. The residue was purified by flash silica gel chromatography (Eluent of 0˜8% ethyl acetate/petroleum ether) to give tert-butyl 4-prop-2-ynoxypiperidine-1-carboxylate (2.4 g, 8.8 mmol, 88.8% yield, 88% purity) as a yellow solid.

MS (ESI) m/z: 184.2 [M-56+H]⁺

¹H NMR (400 MHz, DMSO-d₆) δ=4.17 (d, J=2.4 Hz, 2H), 3.67-3.56 (m, 3H), 3.38 (t, J=2.4 Hz, 1H), 3.02 (t, J=9.6 Hz, 2H), 1.82-1.74 (m, 2H), 1.41-1.37 (m, 9H), 1.35-1.27 (m, 2H)

### Step B. Procedure for Preparation of tert-butyl 4-[3-(4-bromo-3-methyl-phenyl)prop-2-ynoxy]piperidine-1-carboxylate

A mixture of tert-butyl 4-prop-2-ynoxypiperidine-1-carboxylate (2 g, 8.36 mmol, 1 equiv.), 1-bromo-4-iodo-2-methyl-benzene (2.48 g, 8.36 mmol, 1 equiv.), TEA (2.54 g, 25.07 mmol, 3.49 mL, 3 equiv.), Pd(PPh₃)₂Cl₂ (586.60 mg, 835.74 μmol, 0.1 equiv.), and CuI (318.33 mg, 1.67 mmol, 0.2 equiv.) in DMF (20 mL) was degassed and purged with N₂ three times, and then the mixture was stirred at 60° C. for 3 hours under N₂ atmosphere. The reaction mixture was quenched by addition H₂O (50 mL) and extracted with EtOAc (50 mL×3). The combined organic layers were washed with saturated salt solution (50 mL×1), dried over Na₂SO₄, filtered, and concentrated under reduced pressure to give a residue. The residue was purified by flash silica gel chromatography (Eluent of 0˜5% Ethylacetate/petroleum ether) to give tert-butyl 4-[3-(4-bromo-3-methyl-phenyl)prop-2-ynoxy]piperidine-1-carboxylate (2.4 g, 4.8 mmol, 57.0% yield, 81% purity) as a yellow oil.

MS (ESI) m/z: 308.1 [M-100+H]⁺

¹H NMR (400 MHz, DMSO-d₆) δ=7.58 (d, J=8.0 Hz, 1H), 7.45 (d, J=1.6 Hz, 1H), 7.20 (dd, J=2.0, 8.4 Hz, 1H), 4.41 (s, 2H), 3.76-3.56 (m, 3H), 3.05 (t, J=10.0 Hz, 2H), 2.33 (s, 3H), 1.90-1.77 (m, 2H), 1.39 (s, 9H), 1.38-1.32 (m, 2H)

### Step C. Procedure for Preparation of tert-butyl 4-[3-(4-bromo-3-methyl-phenyl)propoxy]piperidine-1-carboxylate

To a solution of tert-butyl 4-[3-(4-bromo-3-methyl-phenyl)prop-2-ynoxy]piperidine-1-carboxylate (2.2 g, 5.39 mmol, 1 equiv.) in EtOAc (20 mL) was added Rh/Al₂O₃ (1.11 g, 538.78 mol, 5% purity, 0.1 equiv.) under N₂ atmosphere. The suspension was degassed and purged with H₂ three times. The mixture was stirred under H₂ (15 psi) at 25° C. for 2 hours. The reaction solution was filtered to give tert-butyl 4-[3-(4-bromo-3-methyl-phenyl) propoxy]piperidine-1-carboxylate (2.2 g, 4.8 mmol, 90.8% yield, 91.7% purity) as a brown oil.

MS (ESI) m/z: 356.2 [M-56+H]⁺

¹H NMR (400 MHz, DMSO-d₆) δ=7.42 (d, J=8.0 Hz, 1H), 7.06 (s, 1H), 6.91-6.85 (m, 1H), 3.81-3.72 (m, 2H), 3.46-3.38 (m, 3H), 3.13-3.03 (m, 2H), 2.63 (t, J=7.6 Hz, 2H), 2.37 (s, 3H), 1.90-1.78 (m, 4H), 1.55-1.48 (m, 2H), 1.47 (s, 9H)

### Step D. Procedure for Preparation of 4-[3-(4-bromo-3-methyl-phenyl)propoxy]piperidine

A mixture of tert-butyl 4-[3-(4-bromo-3-methyl-phenyl)propoxy]piperidine-1-carboxylate (2 g, 4.85 mmol, 1 equiv.) in HCl/EtOAc (20 mL) was degassed and purged with N₂ three times, and then the mixture was stirred at 25° C. for 2 hours under N₂ atmosphere. The reaction mixture was concentrated under reduced pressure to give 4-[3-(4-bromo-3-methyl-phenyl)propoxy]piperidine (2 g, crude) as a brown solid.

¹H NMR (400 MHz, DMSO-d₆) δ=7.43 (d, J=8.0 Hz, 1H), 7.05 (d, J=1.6 Hz, 1H), 6.86 (dd, J=2.0, 8.0 Hz, 1H), 3.61 (s, 1H), 3.40 (t, J=6.2 Hz, 2H), 3.35-3.14 (m, 4H), 2.66-2.57 (m, 2H), 2.38 (s, 3H), 2.13 (s, 1H), 2.10-2.06 (m, 2H), 1.99-1.91 (m, 2H), 1.90-1.81 (m, 2H)

### Step E. Procedure for Preparation of ethyl 2-[4-[3-(4-bromo-3-methyl-phenyl)propoxy]-1-piperidyl]acetate

A mixture of 4-[3-(4-bromo-3-methyl-phenyl)propoxy]piperidine (2 g, 5.74 mmol, 1 equiv., HCl), ethyl 2-bromoacetate (862.05 mg, 5.16 mmol, 571.27 μL, 0.9 equiv.) and K₂CO₃ (2.38 g, 17.21 mmol, 3 equiv.) in CH₃CN (20 mL) was degassed and purged with N₂ three times, and then the mixture was stirred at 40° C. for 4 hours under N₂ atmosphere. The reaction mixture was filtered and concentrated under reduced pressure to give a residue. The residue was purified by flash silica gel chromatography (Eluent of 0˜28% Ethylacetate/petroleum ether) to give ethyl 2-[4-[3-(4-bromo-3-methyl-phenyl)propoxy]-1-piperidyl]acetate (1.8 g, 4.3 mmol, 74.8% yield, 94.9% purity) as a yellow oil.

MS (ESI) m/z: 398.2 [M+H]⁺

¹H NMR (400 MHz, DMSO-d₆) δ=7.42 (d, J=8.0 Hz, 1H), 7.07 (d, J=1.6 Hz, 1H), 6.88 (dd, J=2.0, 8.0 Hz, 1H), 4.19 (q, J=7.2 Hz, 2H), 3.41 (t, J=6.4 Hz, 2H), 3.33-3.26 (m, 1H), 3.22 (s, 2H), 2.87-2.77 (m, 2H), 2.62 (t, J=7.6 Hz, 2H), 2.41-2.32 (m, 4H), 1.98-1.81 (m, 4H), 1.71-1.65 (m, 2H), 1.28 (t, J=7.2 Hz, 3H)

### Step F. Procedure for Preparation of tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate

A mixture of tert-butyl 6-[8-(1,3-benzothiazol-2-ylcarbamoyl)-3,4-dihydro-1H-isoquinolin-2-yl]-3-bromo-pyridine-2-carboxylate (1.5 g, 2.65 mmol, 1 equiv.), 4,4,5,5-tetramethyl-1,3,2-dioxaborolane (1.70 g, 13.26 mmol, 1.92 mL, 5 equiv.), N,N-diethylethanamine (805.25 mg, 7.96 mmol, 1.11 mL, 3 equiv.), and cyclopentyl(diphenyl)phosphane; dichloropalladium; iron (194.09 mg, 265.26 umol, 0.1 equiv.) in THE (3 mL) and CH₃CN (10 mL) was degassed and purged with N₂ three times, and then the mixture was stirred at 100° C. for 1 hour under N₂ atmosphere in microwave. The reaction was filtered to give tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (2 g, 3.2 mmol, 41.0% yield) as a white solid.

MS (ESI) m/z: 613.3 [M+H]⁺

### Step G. Procedure for Preparation of tert-butyl 6-[8-(1,3-benzothiazol-2-ylcarbamoyl)-3,4-dihydro-1H-isoquinolin-2-yl]-3-[4-[3-[[1-(2-ethoxy-2-oxo-ethyl)-4-piperidyl]oxy]propyl]-2-methyl-phenyl]pyridine-2-carboxylate

A mixture of ethyl 2-[4-[3-(4-bromo-3-methyl-phenyl)propoxy]-1-piperidyl]acetate (400 mg, 1.00 mmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (738.13 mg, 1.21 mmol, 1.2 equiv.), Ad₂nBuP Pd G₃ (73.13 mg, 100.42 μmol, 0.1 equiv.), and K₂CO₃ (416.35 mg, 3.01 mmol, 3 equiv.) in dioxane (5 mL) and H₂O (2 mL) was degassed and purged with N₂ three times, and then the mixture was stirred at 100° C. for 1 hour under N₂ atmosphere in microwave. The reaction mixture was concentrated under reduced pressure to give a residue. The residue was purified by flash silica gel chromatography (Eluent of 0-51% ethyl acetate/petroleum ether) to give tert-butyl 6-[8-(1,3-benzothiazol-2-ylcarbamoyl)-3,4-dihydro-1H-isoquinolin-2-yl]-3-[4-[3-[[1-(2-ethoxy-2-oxo-ethyl)-4-piperidyl]oxy]propyl]-2-methyl-phenyl]pyridine-2-carboxylate (500 mg, 590.8 μmol, 58.8% yield, 95% purity) as a yellow oil.

MS (ESI) m/z: 403.0 [M/2+H]⁺

### Step H. Procedure for Preparation of 2-[4-[3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenyl]propoxy]-1-piperidyl]acetic acid

A mixture of tert-butyl 6-[8-(1,3-benzothiazol-2-ylcarbamoyl)-3,4-dihydro-1H-isoquinolin-2-yl]-3-[4-[3-[[1-(2-ethoxy-2-oxo-ethyl)-4-piperidyl]oxy]propyl]-2-methyl-phenyl]pyridine-2-carboxylate (200 mg, 248.75 μmol, 1 equiv.), LiOH·H₂O (41.75 mg, 995.02 μmol, 4 equiv.) in THE (8 mL) and H₂O (2 mL) was degassed and purged with N₂ three times, and then the mixture was stirred at 25° C. for 12 hours under N₂ atmosphere. The reaction mixture was concentrated under reduced pressure to remove THF. The residue was diluted with H₂O (5 mL) and adjusted to pH=4-5 with HCl. Then the mixture was extracted with EtOAc (50 mL×3). The combined organic layers were washed with saturated salt solution (50 mL×1), dried over Na₂SO₄, filtered, and concentrated under reduced pressure to give 2-[4-[3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenyl]propoxy]-1-piperidyl]acetic acid (260 mg, crude) as a yellow solid.

MS (ESI) m/z: 776.6 [M+H]⁺

### Step L Procedure for preparation of tert-butyl 6-[8-(1,3-benzothiazol-2-ylcarbamoyl)-3,4-dihydro-1H-isoquinolin-2-yl]-3-[4-[3-[[1-[2-[[3-(2,6-dioxo-3-piperidyl)-1-methyl-indazol-6-yl]amino]-2-oxo-ethyl]-4-piperidyl]oxy]propyl]-2-methyl-phenyl]pyridine-2-carboxylate

A mixture of 2-[4-[3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenyl]propoxy]-1-piperidyl]acetic acid (200.0 mg, 257.75 μmol, 1 equiv.), 3-(6-amino-1-methyl-indazol-3-yl)piperidine-2,6-dione (79.88 mg, 309.30 μmol, 1.2 equiv.), EDCI (74.12 mg, 386.62 μmol, 1.5 equiv.) in pyridine (2 mL) was degassed and purged with N₂ for three times, and then the mixture was stirred at 25° C. for 2 hours under N₂ atmosphere. The reaction mixture was quenched by addition H₂O (20 mL) and extracted with EtOAc (20 mL×5). The combined organic layers were washed with saturated salt solution (20 mL×1), dried over Na₂SO₄, filtered, and concentrated under reduced pressure to give tert-butyl 6-[8-(1,3-benzothiazol-2-ylcarbamoyl)-3,4-dihydro-1H-isoquinolin-2-yl]-3-[4-[3-[[1-[2-[[3-(2,6-dioxo-3-piperidyl)-1-methyl-indazol-6-yl]amino]-2-oxo-ethyl]-4-piperidyl]oxy]propyl]-2-methyl-phenyl]pyridine-2-carboxylate (500 mg, crude) as a brown oil.

MS (ESI) m/z: 1016.6 [M+H]⁺

### Step J. Procedure for Preparation of 6-[8-(1,3-benzothiazol-2-ylcarbamoyl)-3,4-dihydro-1H-isoquinolin-2-yl]-3-[4-[3-[[1-[2-[[3-(2,6-dioxo-3-piperidyl)-1-methyl-indazol-6-yl]amino]-2-oxo-ethyl]-4-piperidyl]oxy]propyl]-2-methyl-phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[4-[3-[[1-[2-[[3-(2,6-dioxo-3-piperidyl)-1-methyl-indazol-6-yl]amino]-2-oxo-ethyl]-4-piperidyl]oxy]propyl]-2-methyl-phenyl]pyridine-2-carboxylate (150 mg, 147.61 μmol, 1 equiv.) in TFA (2 mL) and DCM (2 mL) was degassed and purged with N₂ three times, and then the mixture was stirred at 40° C. for 2 hours under N₂ atmosphere. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[4-[3-[[1-[2-[[3-(2,6-dioxo-3-piperidyl)-1-methyl-indazol-6-yl]amino]-2-oxo-ethyl]-4-piperidyl]oxy]propyl]-2-methyl-phenyl]pyridine-2-carboxylic acid (46.3 mg, 47.3 μmol, 32.0% yield, 98.9% purity) as a yellow solid.

MS (ESI) m/z: 960.7 [M+H]⁺

¹H NMR (400 MHz, DMSO-d₆) δ=10.88 (s, 1H), 9.87 (s, 1H), 8.07-8.00 (m, 2H), 7.79 (d, J=8.4 Hz, 1H), 7.63 (d, J=8.8 Hz, 2H), 7.51-7.42 (m, 3H), 7.40-7.31 (m, 2H), 7.23 (d, J=8.8 Hz, 1H), 7.05 (s, 1H), 7.01-6.89 (m, 3H), 4.97 (s, 2H), 4.32 (dd, J=4.8, 9.2 Hz, 1H), 3.92 (s, 3H), 3.43 (t, J=6.4 Hz, 4H), 3.15 (s, 2H), 3.02 (t, J=5.6 Hz, 2H), 2.78 (dd, J=4.4, 6.4 Hz, 2H), 2.67 (s, 1H), 2.62-2.57 (m, 5H), 2.32 (d, J=5.2 Hz, 2H), 2.21-2.13 (m, 1H), 2.04 (s, 3H), 1.87 (d, J=9.6 Hz, 2H), 1.76 (s, 2H), 1.63-1.51 (m, 2H)

### Example 84. Preparation of Compound 122

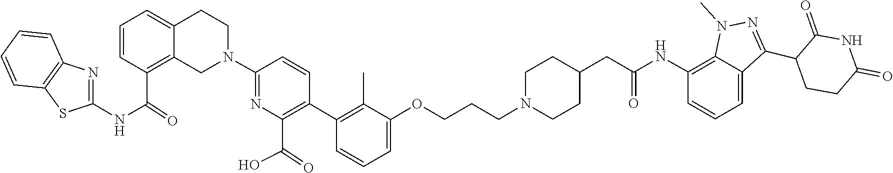 [SMILES: Cc1c(OCCCN2CCC(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[4-[2-[[3-(2,6-dioxo-3-piperidyl)-1-methyl-indazol-7-yl]amino]-2-oxo-ethyl]-1-piperidyl]propoxy]-2-methyl-phenyl]pyridine-2-carboxylic acid

### Step A. Procedure for Preparation of tert-butyl 6-[8-(1,3-benzothiazol-2-ylcarbamoyl)-3,4-dihydro-1H-isoquinolin-2-yl]-3-[3-[3-[4-[2-[[3-(2,6-dioxo-3-piperidyl)-1-methyl-indazol-7-yl]amino]-2-oxo-ethyl]-1-piperidyl]propoxy]-2-methyl-phenyl]pyridine-2-carboxylate

A mixture of 2-[1-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-4-piperidyl]acetic acid (107.79 mg, 138.92 μmol, 1 equiv.), 3-(7-amino-1-methyl-indazol-3-yl)piperidine-2,6-dione (71.76 mg, 277.84 μmol, 2.0 equiv.), HATU (105.64 mg, 277.84 μmol, 2.0 equiv.), and DIEA (17.95 mg, 138.92 μmol, 24.20 μL, 1 equiv.) in DMF (2 mL) was stirred at 25° C. for 12 hours. The reaction mixture was diluted with water (10 mL), filtered, and concentrated under reduced pressure to give the crude product tert-butyl 6-[8-(1,3-benzothiazol-2-ylcarbamoyl)-3,4-dihydro-1H-isoquinolin-2-yl]-3-[3-[3-[4-[2-[[3-(2,6-dioxo-3-piperidyl)-1-methyl-indazol-7-yl]amino]-2-oxo-ethyl]-1-piperidyl]propoxy]-2-methyl-phenyl]pyridine-2-carboxylate (90 mg, 88.5 μmol, 63.7% yield) as a yellow solid.

MS (ESI) m/z: 508.8 [M12+H]⁺

### Step B. Procedure for Preparation of 6-[8-(1,3-benzothiazol-2-ylcarbamoyl)-3,4-dihydro-1H-isoquinolin-2-yl]-3-[3-[3-[4-[2-[[3-(2,6-dioxo-3-piperidyl)-1-methyl-indazol-7-yl]amino]-2-oxo-ethyl]-1-piperidyl]propoxy]-2-methyl-phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[3-[3-[4-[2-[[3-(2,6-dioxo-3-piperidyl)-1-methyl-indazol-7-yl]amino]-2-oxo-ethyl]-1-piperidyl]propoxy]-2-methyl-phenyl]pyridine-2-carboxylate (90 mg, 88.56 μmol, 1 equiv.) in TFA (1 mL) and DCM (1 mL) was stirred at 40° C. for 4 hours. The reaction mixture was concentrated under reduced pressure to give the crude product. The crude product was purified by prep-HPLC to give compound 6-[8-(1,3-benzothiazol-2-ylcarbamoyl)-3,4-dihydro-1H-isoquinolin-2-yl]-3-[3-[3-[4-[2-[[3-(2,6-dioxo-3-piperidyl)-1-methyl-indazol-7-yl]amino]-2-oxo-ethyl]-1-piperidyl]propoxy]-2-methyl-phenyl]pyridine-2-carboxylic acid (25 mg, 24.4 μmol, 27.6% yield, 93.9% purity) as a white solid.

MS (ESI) m/z: 960.3 [M+H]⁺

¹H NMR (400 MHz, DMSO-d₆) δ=10.91 (s, 1H), 9.86 (s, 1H), 8.03 (d, J=8.0 Hz, 1H), 7.79 (d, J=8.0 Hz, 1H), 7.61 (dd, J=7.6, 13.2 Hz, 2H), 7.45-7.35 (m, 6H), 7.11-7.06 (m, 3H), 6.94-6.87 (m, 2H), 6.65 (d, J=7.6 Hz, 1H), 4.98 (s, 2H), 4.36 (dd, J=5.2, 10.4 Hz, 1H), 4.05 (s, 3H), 3.94-3.90 (m, 5H), 3.05-2.94 (m, 6H), 2.73-2.58 (m, 3H), 2.36-2.32 (m, 3H), 2.22-2.00 (m, 4H), 1.91 (s, 3H), 1.74 (d, J=12.4 Hz, 2H), 1.33-1.29 (m, 2H)

### Example 85. Preparation of Compound 145

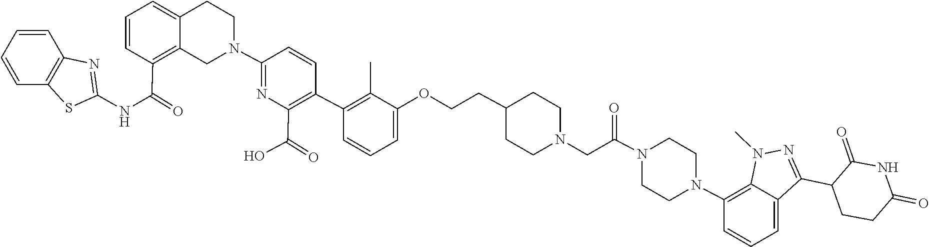 [SMILES: Cc1c(OCCC2CCN(CC(=O)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(2-(1-(2-(4-(3-(2,6-dioxopiperidin-3-yl)-1-methyl-1H-indazol-7-yl)piperazin-1-yl)-2-oxoethyl)piperidin-4-yl)ethoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(2-(1-(2-(4-(3-(2,6-dioxopiperidin-3-yl)-1-methyl-1H-indazol-7-yl)piperazin-1-yl)-2-oxoethyl)piperidin-4-yl)ethoxy)-2-methylphenyl)picolinate

To a solution of 2-(4-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethyl)piperidin-1-yl)acetic acid (100 mg, 131.25 μmol, 1.00 equiv.) in DMF (1 mL) was added DIEA (50.89 mg, 393.74 μmol, 68.58 μL, 3.00 equiv.), HATU (59.88 mg, 157.50 μmol, 1.20 equiv.), and 3-(1-methyl-7-piperazin-1-yl-indazol-3-yl)piperidine-2,6-dione (52.53 mg, 144.37 μmol, 1.10 equiv.). The mixture was stirred at 25° C. for 1 hour. The reaction was diluted with water (20 mL) and extracted with DCM (30 mL×3). The combined organic layers were washed with brine (20 mL×1), dried over Na₂SO₄, filtered, and concentrated under reduced pressure to give tert-butyl 6-(8-(benzo[d]thiazol-2-ylcarbamoyl)-3,4-dihydroisoquinolin-2(1H)-yl)-3-(3-(2-(1-(2-(4-(3-(2,6-dioxopiperidin-3-yl)-1-methyl-1H-indazol-7-yl)piperazin-1-yl)-2-oxoethyl)piperidin-4-yl)ethoxy)-2-methylphenyl)picolinate (130 mg, 119.8 μmol, 91.3% yield) as a yellow solid.

MS (ESI) m/z: 1071.8 [M+H]⁺.

### Step B. Procedure for Preparation of 6-(8-(benzo[d]thiazol-2-ylcarbamoyl)-3,4-dihydroisoquinolin-2(1H)-yl)-3-(3-(2-(1-(2-(4-(3-(2,6-dioxopiperidin-3-yl)-1-methyl-1H-indazol-7-yl)piperazin-1-yl)-2-oxoethyl)piperidin-4-yl)ethoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(2-(1-(2-(4-(3-(2,6-dioxopiperidin-3-yl)-1-methyl-1H-indazol-7-yl)piperazin-1-yl)-2-oxoethyl)piperidin-4-yl)ethoxy)-2-methylphenyl)picolinate (130 mg, 121.35 μmol, 1.00 equiv.) in DCM (1 mL) was added TFA (1.67 g, 14.63 mmol, 1.0 mL, 120.57 equiv.). The mixture was stirred at 40° C. for 1 hour. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(2-(1-(2-(4-(3-(2,6-dioxopiperidin-3-yl)-1-methyl-1H-indazol-7-yl)piperazin-1-yl)-2-oxoethyl)piperidin-4-yl)ethoxy)-2-methylphenyl)picolinic acid (20.6 mg, 18.4 μmol, 15.1% yield, 95.6% purity) as a white solid.

MS (ESI) m/z: 1015.6 [M+H]⁺.

¹H NMR (400 MHz, DMSO-d₆) δ=10.89 (s, 1H), 8.06-7.99 (m, 1H), 7.78 (d, J=8.8 Hz, 1H), 7.63 (d, J=7.6 Hz, 1H), 7.52-7.28 (m, 7H), 7.10-7.00 (m, 3H), 6.94-6.85 (m, 2H), 6.65 (d, J=7.2 Hz, 1H), 5.02-4.96 (m, 2H), 4.37-4.33 (m, 1H), 4.28 (s, 3H), 4.00 (t, J=6.0 Hz, 2H), 3.94-3.90 (m, 2H), 3.06-3.00 (m, 4H), 2.87-2.80 (m, 4H), 2.64-2.59 (m, 6H), 2.21-2.13 (m, 2H), 1.98 (d, J=10.4 Hz, 2H), 1.89 (s, 3H), 1.74-1.67 (m, 4H), 1.52-1.47 (m, 1H), 1.28-1.20 (m, 4H)

### Example 86. Preparation of Compound 146b

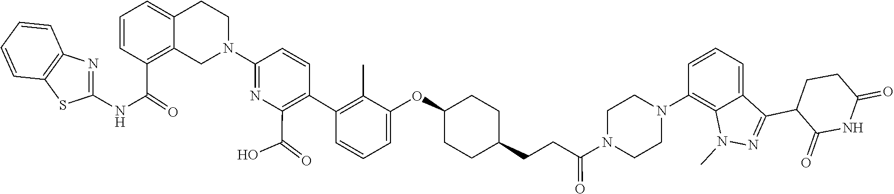 [SMILES: Cc1c(O[C@H]2CC[C@@H](CCC(=O)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4s)-4-(3-(4-(3-(2,6-dioxopiperidin-3-yl)-1-methyl-1H-indazol-7-yl)piperazin-1-yl)-3-oxopropyl)cyclohexyl)oxy)-2-methylphenyl)picolinic acid

### Step A. Procedure for Preparation of tert-butyl 6-[8-(1, 3-benzothiazol-2-ylcarbamoyl)-3, 4-dihydro-1H-isoquinolin-2-yl]-3-[3-[4-(3-ethoxy-3-oxo-propyl) cyclohexoxy]-2-methyl-phenyl]pyridine-2-carboxylate

tert-butyl 6-[8-(1, 3-benzothiazol-2-ylcarbamoyl)-3, 4-dihydro-1H-isoquinolin-2-yl]-3-(4, 4, 5, 5-tetramethyl-1, 3, 2-dioxaborolan-2-yl) pyridine-2-carboxylate (737.46 mg, 1.20 mmol, 1.3 equiv.), ethyl 3-[4-(3-bromo-2-methyl-phenoxy) cyclohexyl]propanoate (400 mg, 926.10 μmol, 85.5% purity, 1 equiv.), KF (161.42 mg, 2.78 mmol, 65.09 μL, 3 equiv.), and [2-(2-aminophenyl) phenyl]palladium (1+); bis (1-adamantyl)-butyl-phosphane; methanesulfonate (134.89 mg, 185.22 mol, 0.2 equiv.) were taken up into a microwave tube in dioxane (4 mL) and H₂O (0.4 mL). The sealed tube was heated at 100° C. for 60 min under microwave. The reaction mixture was filtered and concentrated under reduced pressure to give a residue. The residue was purified by flash silica gel chromatography (Eluent of 0˜50% ethyl acetate/petroleum ether). The compound tert-butyl 6-[8-(1, 3-benzothiazol-2-ylcarbamoyl)-3, 4-dihydro-1H-isoquinolin-2-yl]-3-[3-[4-(3-ethoxy-3-oxo-propyl) cyclohexoxy]-2-methyl-phenyl]pyridine-2-carboxylate (630 mg, 734.1 μmol, 79.3% yield, 90.3% purity) was obtained as a yellow oil.

MS (ESI) m/z: 775.6 [M+H]⁺.

### Step B. Procedure for Preparation of 3-[4-[3-[6-[8-(1, 3-benzothiazol-2-ylcarbamoyl)-3, 4-dihydro-1H-isoquinolin-2-yl]-2-tert-butoxycarbonyl-3-pyridyl]-2-methyl-phenoxy]cyclohexyl]propanoic acid

To a solution of tert-butyl 6-[8-(1, 3-benzothiazol-2-ylcarbamoyl)-3, 4-dihydro-1H-isoquinolin-2-yl]-3-[3-[4-(3-ethoxy-3-oxo-propyl) cyclohexoxy]-2-methyl-phenyl]pyridine-2-carboxylate (600 mg, 774.23 μmol, 1 equiv.) in THE (6 mL) and H₂O (0.6 mL) was added LiOH·H₂O (97.47 mg, 2.32 mmol, 3 equiv.). The mixture was stirred at 25° C. for 40 hours. The reaction mixture was concentrated under reduced pressure to give a residue. The residue was treated with water (4 mL). The pH of the mixture was adjusted to around 3 by progressively adding diluted HCl. The mixture was filtered to give 3-[4-[3-[6-[8-(1, 3-benzothiazol-2-ylcarbamoyl)-3, 4-dihydro-1H-isoquinolin-2-yl]-2-tert-butoxycarbonyl-3-pyridyl]-2-methyl-phenoxy]cyclohexyl]propanoic acid (557 mg, 618.96 μmol, 79.95% yield, 83% purity) as a yellow solid.

MS (ESI) m/z: 747.6 [M+H]⁺.

### Step C. Procedure for Preparation of tert-butyl 6-[8-(1, 3-benzothiazol-2-ylcarbamoyl)-3, 4-dihydro-1H-isoquinolin-2-yl]-3-[3-[4-[3-[4-[3-(2, 6-dioxo-3-piperidyl)-1-methyl-indazol-7-yl]piperazin-1-yl]-3-oxo-propyl] cyclohexoxy]-2-methyl-phenyl]pyridine-2-carboxylate

To a solution of 3-[4-[3-[6-[8-(1, 3-benzothiazol-2-ylcarbamoyl)-3, 4-dihydro-1H-isoquinolin-2-yl]-2-tert-butoxycarbonyl-3-pyridyl]-2-methyl-phenoxy]cyclohexyl]propanoic acid (200 mg, 267.77 μmol, 1 equiv.) in DMF (1 mL) was added HATU (152.72 mg, 401.65 μmol, 1.5 equiv.), DIPEA (103.82 mg, 803.31 μmol, 139.92 μL, 3 equiv.), and 3-(1-methyl-7-piperazin-1-yl-indazol-3-yl) piperidine-2, 6-dione (131.49 mg, 401.65 μmol, 1.5 equiv.) at 25° C. The reaction mixture was stirred at 25° C. for 16 hours. The mixture was triturated with water (5 mL) and filtered to give a residue. The compound tert-butyl 6-[8-(1, 3-benzothiazol-2-ylcarbamoyl)-3, 4-dihydro-1H-isoquinolin-2-yl]-3-[3-[4-[3-[4-[3-(2, 6-dioxo-3-piperidyl)-1-methyl-indazol-7-yl]piperazin-1-yl]-3-oxo-propyl]cyclohexoxy]-2-methyl-phenyl]pyridine-2-carboxylate (270 mg, 180.9 μmol, 67.5% yield, 70.8% purity) was obtained as a yellow solid.

MS (ESI) m/z: 1056.4 [M+H]⁺.

### Step D. Procedure for Preparation of 6-(8-(benzo[d]thiazol-2-ylcarbamoyl)-3,4-dihydroisoquinolin-2(1H)-yl)-3-(3-(((1s,4s)-4-(3-(4-(3-(2,6-dioxopiperidin-3-yl)-1-methyl-1H-indazol-7-yl)piperazin-1-yl)-3-oxopropyl)cyclohexyl)oxy)-2-methylphenyl)picolinic acid

A solution of tert-butyl 6-[8-(1, 3-benzothiazol-2-ylcarbamoyl)-3, 4-dihydro-1H-isoquinolin-2-yl]-3-[3-[4-[3-[4-[3-(2, 6-dioxo-3-piperidyl)-1-methyl-indazol-7-yl]piperazin-1-yl]-3-oxo-propyl]cyclohexoxy]-2-methyl-phenyl]pyridine-2-carboxylate (240 mg, 227.21 μmol, 1 eq) in DCM (1 mL) and TFA (1 mL) was stirred at 25° C. for 16 hours. The reaction mixture was filtered and concentrated under reduced pressure to give a residue. The residue was purified by prep-HPLC. The compound 6-(8-(benzo[d]thiazol-2-ylcarbamoyl)-3,4-dihydroisoquinolin-2(1H)-yl)-3-(3-(((1s,4s)-4-(3-(4-(3-(2,6-dioxopiperidin-3-yl)-1-methyl-1H-indazol-7-yl)piperazin-1-yl)-3-oxopropyl)cyclohexyl)oxy)-2-methylphenyl)picolinic acid (67.2 mg, 66.5 μmol, 29.2% yield, 98.9% purity) was obtained as a white solid.

MS (ESI) m/z: 1000.4 [M+H]⁺.

¹H NMR (400 MHz, DMSO-d₆) δ=12.99-12.23 (m, 2H), 10.89 (s, 1H), 8.03 (d, J=8.0 Hz, 1H), 7.79 (d, J=8.0 Hz, 1H), 7.62 (d, J=7.6 Hz, 1H), 7.52-7.31 (m, 6H), 7.06 (d, J=8.0 Hz, 1H), 7.04-6.99 (m, 2H), 6.96 (d, J=8.8 Hz, 1H), 6.89 (d, J=8.4 Hz, 1H), 6.60 (d, J=7.2 Hz, 1H), 4.98 (s, 2H), 4.61 (s, 1H), 4.53-4.40 (m, 1H), 4.34 (dd, J=5.2, 9.6 Hz, 1H), 4.26 (s, 3H), 3.91 (t, J=5.6 Hz, 3H), 3.25-3.10 (m, 2H), 3.02 (t, J=5.6 Hz, 2H), 2.72-2.52 (m, 4H), 2.47-2.26 (m, 4H), 2.18 (d, J=5.4 Hz, 1H), 2.01-1.84 (m, 5H), 1.67-1.18 (m, 10H)

### Example 87. Preparation of Compound 150

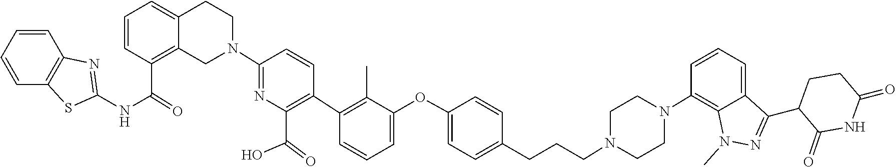 [SMILES: Cc1c(Oc2ccc(CCCN3CCN(c4cccc5c(C6CCC(=O)NC6=O)nn(C)c45)CC3)cc2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-7-yl]piperazin-1-yl]propyl]phenoxy]-2-methyl-phenyl]pyridine-2-carboxylic acid

### Step A. Procedure for Preparation of tert-butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-7-yl]piperazin-1-yl]propyl]phenoxy]-2-methyl-phenyl]pyridine-2-carboxylate

A mixture of tert-butyl 6-(8-(benzo[d]thiazol-2-ylcarbamoyl)-3,4-dihydroisoquinolin-2(1H)-yl)-3-(2-methyl-3-(4-(3-oxopropyl)phenoxy)phenyl)picolinate (100 mg, 137.96 μmol, 1.0 equiv.), 3-(1-methyl-7-piperazin-1-yl-indazol-3-yl)piperidine-2,6-dione (54.20 mg, 165.55 μmol, 1.2 equiv.), HCOOH (6.63 mg, 137.96 μmol, 1.0 equiv.), and NaBH(OAc)₃ (43.86 mg, 206.94 μmol, 1.5 equiv.) in DCM (2 mL) was degassed and purged with N₂ three times, and then the mixture was stirred at 25° C. for 15 hours under N₂ atmosphere. The reaction mixture was concentrated under reduced pressure to give a residue. The compound tert-butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-7-yl]piperazin-1-yl]propyl]phenoxy]-2-methyl-phenyl]pyridine-2-carboxylate (150 mg, crude) was obtained as a yellow oil.

MS (ESI) m/z: 1036.7 [M+H]⁺.

### Step B. Procedure for Preparation of 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-7-yl]piperazin-1-yl]propyl]phenoxy]-2-methyl-phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-7-yl]piperazin-1-yl]propyl]phenoxy]-2-methyl-phenyl]pyridine-2-carboxylate (150 mg, 144.75 μmol, 1 equiv.) in TFA (1 mL) and DCM (1 mL) was stirred at 40° C. for 21 hours under N₂ atmosphere. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-7-yl]piperazin-1-yl]propyl]phenoxy]-2-methyl-phenyl]pyridine-2-carboxylic acid (30.1 mg, 29.9 μmol, 20.6% yield, 97.4% purity) as a white solid.

MS (ESI) m/z: 981.0 [M+H]⁺.

¹H NMR (400 MHz, DMSO-d₆) δ=10.89 (s, 1H), 8.15 (s, 1H), 8.03 (d, J=8.0 Hz, 1H), 7.79 (d, J=8.0 Hz, 1H), 7.63 (d, J=7.6 Hz, 1H), 7.53 (d, J=8.8 Hz, 1H), 7.50-7.31 (m, 6H), 7.21-7.15 (m, 3H), 7.05-6.98 (m, 3H), 6.89 (d, J=7.6 Hz, 1H), 6.86-6.80 (m, 3H), 5.00 (s, 2H), 4.34 (dd, J=5.2, 9.6 Hz, 1H), 4.23 (s, 3H), 3.93 (t, J=6.0 Hz, 2H), 3.04 (d, J=5.6 Hz, 2H), 2.98-2.84 (m, 4H), 2.71-2.55 (m, 7H), 2.40 (t, J=6.8 Hz, 2H), 2.36-2.28 (m, 2H), 2.21-2.13 (m, 1H), 1.91 (s, 3H), 1.81-1.74 (m, 2H)

### Example 88. Preparation of Compound 162b

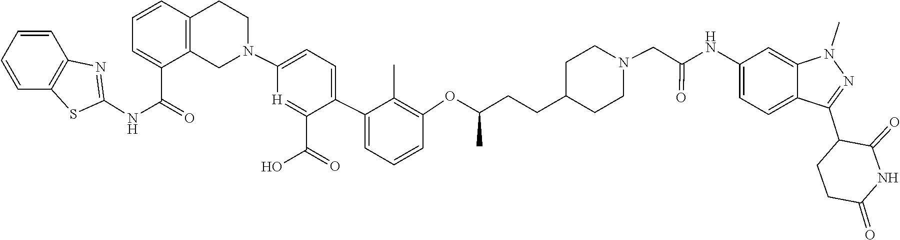

### 6-[8-(1,3-benzothiazol-2-ylcarbamoyl)-3,4-dihydro-1H-isoquinolin-2-yl]-3-[3-[(1R)-3-[1-[2-[[3-(2,6-dioxo-3-piperidyl)-1-methyl-indazol-6-yl]amino]-2-oxo-ethyl]-4-piperidyl]-1-methyl-propoxy]-2-methyl-phenyl]pyridine-2-carboxylic acid

### Step A. Procedure for Preparation of tert-butyl 4-[(3R)-3-(3-bromo-2-methyl-phenoxy)butyl]piperidine-1-carboxylate

A mixture of tert-butyl 4-[(3S)-3-hydroxybutyl]piperidine-1-carboxylate (1.08 g, 4.20 mmol, 1 equiv.), 3-bromo-2-methyl-phenol (784.85 mg, 4.20 mmol, 1 equiv.), and 2-(tributyl-λ⁵-phosphanylidene)acetonitrile (1.22 g, 5.04 mmol, 1.2 equiv.) in toluene (2 mL) was degassed and purged with N₂ three times, and then the mixture was stirred at 120° C. for 1 hour under N₂ atmosphere. The reaction mixture was concentrated under reduced pressure to give a residue. The residue was purified by column chromatography (SiO₂, petroleum ether/ethyl acetate=10/1 to 3/1) to give tert-butyl 4-[(3R)-3-(3-bromo-2-methyl-phenoxy)butyl]piperidine-1-carboxylate (1.3 g, 3.0 mmol, 72.6% yield) as a colorless oil.

¹H NMR (400 MHz, CDCl₃) δ=7.13 (t, J=8.4 Hz, 1H), 6.98 (t, J=8.0 Hz, 1H), 6.76 (d, J=8.4 Hz, 1H), 4.36-4.25 (m, 1H), 4.16-4.01 (m, 2H), 2.68 (t, J=12.4 Hz, 2H), 2.30 (s, 3H), 1.80-1.58 (m, 5H), 1.47 (s, 9H), 1.44-1.35 (m, 2H), 1.29 (d, J=6.0 Hz, 3H), 1.16-1.05 (m, 2H).

### Step B. Procedure for Preparation of 4-[(3R)-3-(3-bromo-2-methyl-phenoxy)butyl]piperidine

A solution of tert-butyl 4-[(3R)-3-(3-bromo-2-methyl-phenoxy)butyl]piperidine-1-carboxylate (1.3 g, 3.05 mmol, 1 equiv.) in HCl/EtOAc (1 mL) was stirred at 25° C. for 12 hours. The reaction mixture was concentrated under reduced pressure to give a residue. The residue was purified by column chromatography (SiO₂, petroleum ether/ethyl acetate=10/1 to 3/1) to give 4-[(3R)-3-(3-bromo-2-methyl-phenoxy)butyl]piperidine (650 mg, 1.9 mmol, 65.3% yield) as a colorless oil.

¹H NMR (400 MHz, CDCl₃) δ=9.59 (s, 1H), 7.13 (d, J=8.0 Hz, 1H), 6.98 (t, J=8.0 Hz, 1H), 6.74 (d, J=8.4 Hz, 1H), 4.39-4.20 (m, 1H), 3.48 (d, J=12.4 Hz, 2H), 2.84 (m, J=12.0 Hz, 2H), 2.28 (s, 3H), 1.90 (d, J=13.6 Hz, 2H), 1.71-1.37 (m, 7H), 1.28 (d, J=6.0 Hz, 3H).

### Step C. Procedure for Preparation of ethyl 2-[4-[(3R)-3-(3-bromo-2-methyl-phenoxy) butyl]-1-piperidyl] acetate

To a solution of 4-[(3R)-3-(3-bromo-2-methyl-phenoxy)butyl]piperidine (650 mg, 1.99 mmol, 1 equiv.), and ethyl 2-bromoacetate (332.70 mg, 1.99 mmol, 220.33 μL, 1 equiv.) in CH₃CN (3 mL) was added K₂CO₃ (275.34 mg, 1.99 mmol, 1 equiv.). The mixture was stirred at 60° C. for 2 hours. The reaction mixture was filtered and concentrated under reduced pressure to give a residue. The residue was purified by column chromatography (SiO₂, petroleum ether/ethyl acetate=10/1 to 2/1) to give ethyl 2-[4-[(3R)-3-(3-bromo-2-methyl-phenoxy) butyl]-1-piperidyl] acetate (600 mg, 1.46 mmol, 73.04% yield) as a yellow oil.

MS (ESI) m/z: 414.2 [M+H]⁺

¹H NMR (400 MHz, CDCl₃) δ=7.13 (d, J=8.0 Hz, 1H), 6.98 (t, J=8.0 Hz, 1H), 6.76 (d, J=8.4 Hz, 1H), 4.35-4.26 (m, 1H), 4.19 (q, J=7.2 Hz, 2H), 3.19 (s, 2H), 2.93 (d, J=11.2 Hz, 2H), 2.30 (s, 3H), 2.13 (t, J=11.2 Hz, 2H), 1.80-1.56 (m, 6H), 1.36-1.26 (m, 9H).

### Step D. Procedure for Preparation of tert-butyl 6-[8-(1,3-benzothiazol-2-ylcarbamoyl)-3,4-dihydro-1H-isoquinolin-2-yl]-3-[3-[(1R)-3-[1-(2-ethoxy-2-oxo-ethyl)-4-piperidyl]-1-methyl-propoxy]-2-methyl-phenyl]pyridine-2-carboxylate

A mixture of ethyl 2-[4-[(3R)-3-(3-bromo-2-methyl-phenoxy)butyl]-1-piperidyl]acetate (700 mg, 1.70 mmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (1.04 g, 1.70 mmol, 1 equiv.), [2-(2-aminophenyl)phenyl]palladium(1+); bis(1-adamantyl)-butyl-phosphane; methanesulfonate (123.63 mg, 169.75 μmol, 0.1 equiv.), KF (1.5 M, 3.40 mL, 3 equiv.) in dioxane (5 mL) and water (1 mL) was degassed and purged with N₂ three times, and then the mixture was stirred at 100° C. for 1 hour under microwave. The reaction mixture was filtered and concentrated under reduced pressure to give a residue. The residue was purified by column chromatography (SiO₂, petroleum ether/ethyl acetate=10/1 to 2/1) to give tert-butyl 6-[8-(1,3-benzothiazol-2-ylcarbamoyl)-3,4-dihydro-1H-isoquinolin-2-yl]-3-[3-[(1R)-3-[1-(2-ethoxy-2-oxo-ethyl)-4-piperidyl]-1-methyl-propoxy]-2-methyl-phenyl]pyridine-2-carboxylate (850 mg, 1.0 mmol, 61.21% yield) as a yellow oil.

MS (ESI) m/z: 818.7 [M+H]⁺

¹H NMR (400 MHz, CDCl₃) δ=7.86 (d, J=8.0 Hz, 1H), 7.63 (d, J=8.0 Hz, 1H), 7.57 (d, J=7.2 Hz, 1H), 7.43-7.28 (m, 5H), 7.08 (t, J=8.0 Hz, 1H), 6.90 (d, J=8.8 Hz, 1H), 6.80 (d, J=8.0 Hz, 1H), 6.67 (d, J=7.6 Hz, 1H), 5.23-4.85 (m, 2H), 4.42-4.29 (m, 1H), 4.14-4.10 (m, 4H), 3.22 (s, 2H), 3.07 (t, J=6.4 Hz, 2H), 3.01-2.88 (m, 2H), 2.25-2.09 (m, 2H), 2.05 (s, 5H), 1.68 (s, 4H), 1.50-1.41 (m, 2H), 1.32 (d, J=5.6 Hz, 3H), 1.26 (d, J=3.2 Hz, 4H), 1.14 (s, 9H).

### Step E. Procedure for Preparation of 2-[4-[(3R)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]butyl]-1-piperidyl]acetic acid

To a solution of tert-butyl 6-[8-(1,3-benzothiazol-2-ylcarbamoyl)-3,4-dihydro-1H-isoquinolin-2-yl]-3-[3-[(1R)-3-[1-(2-ethoxy-2-oxo-ethyl)-4-piperidyl]-1-methyl-propoxy]-2-methyl-phenyl]pyridine-2-carboxylate (200 mg, 244.49 μmol, 1 equiv.) in THE (0.5 mL), H₂O (0.5 mL) was added LiOH·H₂O (51.30 mg, 1.22 mmol, 5 equiv.). The mixture was stirred at 25° C. for 2 hours. The pH was adjusted to 6 with 1M HCl. The residue was extracted with DCM (2 mL×3). The combined organic layers were filtered and concentrated under reduced pressure to give a residue. The residue was purified by column chromatography (SiO₂, petroleum ether/ethyl acetate=10/1 to 3/1) to give 2-[4-[(3R)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]butyl]-1-piperidyl]acetic acid (160 mg, 202.54 μmol, 82.84% yield) as a yellow solid.

MS (ESI) m/z: 790.7 [M+H]⁺

### Step F. Procedure for Preparation of tert-butyl 6-[8-(1,3-benzothiazol-2-ylcarbamoyl)-3,4-dihydro-1H-isoquinolin-2-yl]-3-[3-[(1R)-3-[1-[2-[[3-(2,6-dioxo-3-piperidyl)-1-methyl-indazol-6-yl]amino]-2-oxo-ethyl]-4-piperidyl]-1-methyl-propoxy]-2-methyl-phenyl]pyridine-2-carboxylate

To a solution of 2-[4-[(3R)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]butyl]-1-piperidyl]acetic acid (80 mg, 101.27 μmol, 1 equiv.), 3-(6-amino-1-methyl-indazol-3-yl)piperidine-2,6-dione (28.77 mg, 111.40 μmol, 1.1 equiv.) in DMF (1 mL) was added HATU (57.76 mg, 151.90 μmol, 1.5 equiv.) and DIEA (39.26 mg, 303.81 μmol, 52.92 μL, 3 equiv.). The mixture was stirred at 25° C. for 2 hours. The mixture was quenched by addition of water (2 mL) at 25° C. when precipitation occurred. The mixture was then filtered and concentrated under reduced pressure to give tert-butyl 6-[8-(1,3-benzothiazol-2-ylcarbamoyl)-3,4-dihydro-1H-isoquinolin-2-yl]-3-[3-[(1R)-3-[1-[2-[[3-(2,6-dioxo-3-piperidyl)-1-methyl-indazol-6-yl]amino]-2-oxo-ethyl]-4-piperidyl]-1-methyl-propoxy]-2-methyl-phenyl]pyridine-2-carboxylate (100 mg, 97.0 μmol, 95.8% yield) as a white solid.

MS (ESI) m/z: 1030.8 [M+H]

### Step G. Procedure for Preparation of 6-[8-(1,3-benzothiazol-2-ylcarbamoyl)-3,4-dihydro-1H-isoquinolin-2-yl]-3-[3-[(1R)-3-[1-[2-[[3-(2,6-dioxo-3-piperidyl)-1-methyl-indazol-6-yl]amino]-2-oxo-ethyl]-4-piperidyl]-1-methyl-propoxy]-2-methyl-phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[(1R)-3-[1-[2-[[3-(2,6-dioxo-3-piperidyl)-1-methyl-indazol-6-yl]amino]-2-oxo-ethyl]-4-piperidyl]-1-methyl-propoxy]-2-methyl-phenyl]pyridine-2-carboxylate (100 mg, 97.06 μmol, 1 equiv.) in DCM (1 mL) and TFA (1 mL) was stirred at 25° C. for 2 hours. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[(1R)-3-[1-[2-[[3-(2,6-dioxo-3-piperidyl)-1-methyl-indazol-6-yl]amino]-2-oxo-ethyl]-4-piperidyl]-1-methyl-propoxy]-2-methyl-phenyl]pyridine-2-carboxylic acid (31.6 mg, 31.2 μmol, 32.1% yield, 95.9% purity) as a white solid.

MS (ESI) m/z: 974.7[M+H]

¹H NMR (400 MHz, DMSO-d₆) δ=13.07-12.65 (s, 1H), 10.87 (s, 1H), 9.99-9.72 (s, 1H), 8.13 (s, 1H), 8.08-7.97 (m, 2H), 7.78 (d, J=8.0 Hz, 1H), 7.66-7.59 (m, 2H), 7.50-7.41 (m, 3H), 7.40-7.31 (m, 2H), 7.20 (d, J=8.4 Hz, 1H), 7.12-7.04 (m, 1H), 6.96 (d, J=8.8 Hz, 1H), 6.89 (d, J=8.8 Hz, 1H), 6.61 (d, J=7.2 Hz, 1H), 4.98 (s, 2H), 4.45-4.37 (m, 1H), 4.32 (dd, J=4.8, 9.6 Hz, 1H), 3.92 (s, 5H), 3.02 (t, J=5.6 Hz, 2H), 2.94-2.85 (m, 2H), 2.70-2.59 (m, 4H), 2.38-2.28 (m, 2H), 2.21-2.13 (m, 2H), 1.88 (s, 3H), 1.71-1.63 (m, 3H), 1.45-1.21 (m, 9H).

### Example 89. Preparation of Compound 164a

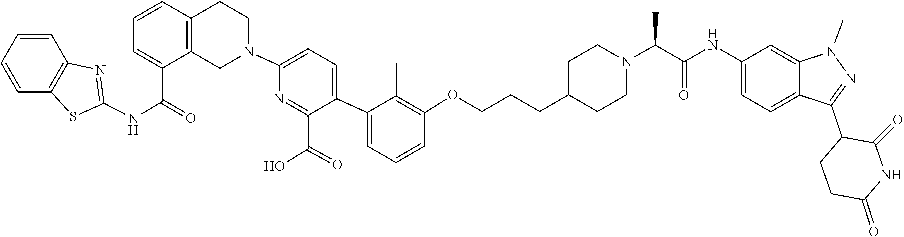 [SMILES: Cc1c(OCCCC2CCN([C@@H](C)C(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-(1-((2S)-1-((3-(2,6-dioxopiperidin-3-yl)-1-methyl-1H-indazol-6-yl)amino)-1-oxopropan-2-yl)piperidin-4-yl)propoxy)-2-methylphenyl)picolinic acid

### Step A. Procedure for Preparation of (S)-tert-butyl 6-(8-(benzo[d]thiazol-2-ylcarbamoyl)-3,4-dihydroisoquinolin-2(1H)-yl)-3-(3-(3-(1-(1-ethoxy-1-oxopropan-2-yl)piperidin-4-yl)propoxy)-2-methylphenyl)picolinate

A mixture of (S)-ethyl 2-(4-(3-(3-bromo-2-methylphenoxy)propyl)piperidin-1-yl)propanoate (450 mg, 1.09 mmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (735.30 mg, 1.20 mmol, 1.1 equiv.) in dioxane (10 mL) was added KF (1.5 M, 2.18 mL, 3 equiv.) and Ad₂nBuP Pd G₃(cataCXium® A Pd G₃) (79.47 mg, 109.13 μmol, 0.1 equiv.). After addition, the mixture was degassed and purged with N₂ three times, and then the mixture was stirred at 100° C. for 2 hours under N₂ atmosphere. The reaction mixture was filtered and concentrated under reduced pressure to give a residue. The residue was purified by flash silica gel chromatography (Eluent of 0˜45% ethyl acetate/petroleum ether) to give (S)-tert-butyl 6-(8-(benzo[d]thiazol-2-ylcarbamoyl)-3,4-dihydroisoquinolin-2(1H)-yl)-3-(3-(3-(1-(1-ethoxy-1-oxopropan-2-yl)piperidin-4-yl)propoxy)-2-methylphenyl)picolinate (600 mg, 690.7 μmol, 63.2% yield, 94.1% purity) as a yellow solid.

MS (ESI) m/z: 818.5 [M+H]⁺

### Step B. Procedure for Preparation of (S)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)propanoic acid

To a solution of (S)-tert-butyl 6-(8-(benzo[d]thiazol-2-ylcarbamoyl)-3,4-dihydroisoquinolin-2(1H)-yl)-3-(3-(3-(1-(1-ethoxy-1-oxopropan-2-yl)piperidin-4-yl)propoxy)-2-methylphenyl)picolinate (600 mg, 733.47 μmol, 1 equiv.) in THE (6 mL) was added LiOH·H₂O (92.34 mg, 2.20 mmol, 3 equiv.) and H₂O (1.5 mL). The mixture was stirred at 40° C. for 16 hours. The reaction mixture was concentrated under reduced pressure to remove THF. The aqueous phase was adjusted to pH=4˜5 with 1M HCl. The reaction mixture was filtered and diluted in ethyl acetate. The combined organic layers were dried over Na₂SO₄, filtered and concentrated under reduced pressure to give (S)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)propanoic acid (600 mg, crude) as a yellow solid.

MS (ESI) m/z: 790.8 [M+H]⁺

### Step C. Procedure for Preparation of tert-butyl 6-(8-(benzo[d]thiazol-2-ylcarbamoyl)-3,4-dihydroisoquinolin-2(1H)-yl)-3-(3-(3-(1-((2S)-1-((3-(2,6-dioxopiperidin-3-yl)-1-methyl-11H-indazol-6-yl)amino)-1-oxopropan-2-yl)piperidin-4-yl)propoxy)-2-methylphenyl)picolinate

To a solution of (S)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)propanoic acid (100 mg, 126.59 μmol, 1 equiv.), 3-(6-amino-1-methyl-1H-indazol-3-yl)piperidine-2,6-dione (39.23 mg, 151.90 μmol, 1.2 equiv.) in pyridine (1 mL) was added EDCI (36.40 mg, 189.88 μmol, 1.5 equiv.). The mixture was stirred at 25° C. for 3 hours. The reaction mixture was quenched by addition H₂O 2 mL. The reaction mixture was filtered and washed with 5 mL of water. After addition, the filter cake was diluted in DCM 10 mL. The combined organic layers were dried over Na₂SO₄, filtered and concentrated under reduced pressure to give tert-butyl 6-(8-(benzo[d]thiazol-2-ylcarbamoyl)-3,4-dihydroisoquinolin-2(1H)-yl)-3-(3-(3-(1-((2S)-1-((3-(2,6-dioxopiperidin-3-yl)-1-methyl-1H-indazol-6-yl)amino)-1-oxopropan-2-yl)piperidin-4-yl)propoxy)-2-methylphenyl)picolinate (100 mg, crude) as a red solid.

MS (ESI) m/z: 1030.8 [M+H]⁺

### Step D. Procedure for Preparation of 6-(8-(benzo[d]thiazol-2-ylcarbamoyl)-3,4-dihydroisoquinolin-2(1H)-yl)-3-(3-(3-(1-((2S)-1-((3-(2,6-dioxopiperidin-3-yl)-1-methyl-11H-indazol-6-yl)amino)-1-oxopropan-2-yl)piperidin-4-yl)propoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(3-(1-((2S)-1-((3-(2,6-dioxopiperidin-3-yl)-1-methyl-1H-indazol-6-yl)amino)-1-oxopropan-2-yl)piperidin-4-yl)propoxy)-2-methylphenyl)picolinate (100 mg, 89.71 μmol, 92.42% purity, 1 equiv.) in DCM (1 mL) was added TFA (1.54 g, 13.51 mmol, 1 mL, 150.56 equiv.). The mixture was stirred at 25° C. for 12 hours. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(3-(1-((2S)-1-((3-(2,6-dioxopiperidin-3-yl)-1-methyl-1H-indazol-6-yl)amino)-1-oxopropan-2-yl)piperidin-4-yl)propoxy)-2-methylphenyl)picolinic acid (63.4 mg, 64.2 μmol, 71.6% yield, 98.6% purity) as a white solid.

MS (ESI) m/z: 974.7 [M+H]⁺.

¹H NMR (400 MHz, DMSO-d₆) δ=13.22-12.35 (m, 1H), 10.88 (s, 1H), 9.92 (s, 1H), 8.14 (s, 1H), 8.08 (s, 1H), 8.03 (d, J=7.6 Hz, 1H), 7.79 (d, J=8.0 Hz, 1H), 7.62 (d, J=8.6 Hz, 2H), 7.51-7.41 (m, 3H), 7.41-7.30 (m, 2H), 7.19 (dd, J=1.6, 7.2 Hz, 1H), 7.14-7.03 (m, 1H), 6.96 (d, J=8.8 Hz, 1H), 6.87 (d, J=8.4 Hz, 1H), 6.62 (d, J=8.4 Hz, 1H), 4.98 (s, 2H), 4.33-4.29 (m, 1H), 3.97-3.88 (m, 7H), 3.02 (t, J=5.6 Hz, 2H), 2.87-2.79 (m, 2H), 2.67-2.59 (m, 2H), 2.38-2.27 (m, 2H), 2.21-2.11 (m, 2H), 1.89 (s, 3H), 1.81-1.65 (m, 4H), 1.43-1.34 (m, 2H), 1.33-1.12 (m, 7H).

### Example 90. Preparation of Compound 168a

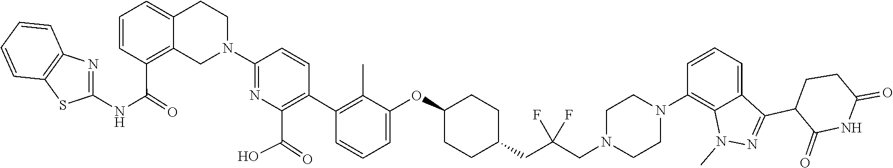 [SMILES: Cc1c(O[C@H]2CC[C@H](CC(F)(F)CN3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-7-yl)piperazin-1-yl)-2,2-difluoropropyl)cyclohexyl)oxy)-2-methylphenyl)picolinic acid

### Step A. Procedure for Preparation of 3-[7-[4-[3-[4-(3-bromo-2-methyl-phenoxy)cyclohexyl]-2,2-difluoro-propyl]piperazin-1-yl]-1-methyl-indazol-3-yl]piperidine-2,6-dione

A mixture of 3-((1r,4r)-4-(3-bromo-2-methylphenoxy)cyclohexyl)-2,2-difluoropropyl trifluoromethanesulfonate (270 mg, 545.13 μmol, 1 equiv.), 3-(1-methyl-7-piperazin-1-yl-indazol-3-yl)piperidine-2,6-dione (151.70 mg, 463.36 μmol, 0.85 equiv.), and K₂CO₃ (188.35 mg, 1.36 mmol, 2.5 equiv.) in CH₃CN (1.5 mL) was stirred at 50° C. for 10 hours. The reaction mixture was filtered and concentrated under reduced pressure to give a residue. The residue was purified by flash silica gel chromatography (Eluent of 0˜ 20% ethyl acetate/petroleum ether) to give 3-[7-[4-[3-[4-(3-bromo-2-methyl-phenoxy)cyclohexyl]-2,2-difluoro-propyl]piperazin-1-yl]-1-methyl-indazol-3-yl]piperidine-2,6-dione (150 mg, 223.0 μmol, 40.9% yield) as a colorless oil.

MS (ESI) m/z: 673.6 [M+H]⁺.

### Step B. Procedure for Preparation of tert-butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-7-yl]piperazin-1-yl]-2,2-difluoro-propyl]cyclohexoxy]-2-methyl-phenyl]pyridine-2-carboxylate

A mixture of tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (191.25 mg, 312.22 μmol, 1.4 equiv.), 3-[7-[4-[3-[4-(3-bromo-2-methyl-phenoxy)cyclohexyl]-2,2-difluoro-propyl]piperazin-1-yl]-1-methyl-indazol-3-yl]piperidine-2,6-dione (150 mg, 223.01 μmol, 1 equiv.), [2-(2-aminophenyl)phenyl]palladium(1+); bis(1-adamantyl)-butyl-phosphane; methanesulfonate (32.48 mg, 44.60 μmol, 0.2 equiv.), and KF (1.5 M, 223.01 uL, 1.5 eq) in dioxane (0.4 mL) was degassed and purged with N₂ three times, and then the mixture was stirred at 100° C. for 1 hour under microwave. The reaction mixture was filtered and concentrated under reduced pressure to give a residue. The residue was purified by flash silica gel chromatography (Eluent of 0˜50% ethyl acetate/petroleum ether) to give tert-butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-7-yl]piperazin-1-yl]-2,2-difluoro-propyl]cyclohexoxy]-2-methyl-phenyl]pyridine-2-carboxylate (200 mg, 185.5 umol, 83.2% yield) as a pale yellow solid.

MS (ESI) m/z: 1079.7 [M+H]⁺.

### Step C. Procedure for Preparation of 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-7-yl)piperazin-1-yl)-2,2-difluoropropyl)cyclohexyl)oxy)-2-methylphenyl)picolinic acid

A solution of tert-butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-7-yl]piperazin-1-yl]-2,2-difluoro-propyl]cyclohexoxy]-2-methyl-phenyl]pyridine-2-carboxylate (200 mg, 185.48 μmol, 1 equiv.) in TFA (0.5 mL) and DCM (0.5 mL) was stirred at 25° C. for 10 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-7-yl)piperazin-1-yl)-2,2-difluoropropyl)cyclohexyl)oxy)-2-methylphenyl)picolinic acid (97.3 mg, 91.9 μmol, 49.6% yield, 96.6% purity) as a off-white solid.

MS (ESI) m/z: 1023.7 [M+H]⁺.

¹H NMR (400 MHz, DMSO-d₆) δ=12.97-12.44 (m, 2H), 10.88 (s, 1H), 8.03 (d, J=8.0 Hz, 1H), 7.79 (d, J=8.0 Hz, 1H), 7.62 (d, J=6.8 Hz, 1H), 7.49-7.33 (m, 6H), 7.13-6.91 (m, 5H), 6.62 (d, J=7.6 Hz, 1H), 5.05-4.94 (m, 2H), 4.36-4.30 (m, 1H), 4.24 (s, 3H), 3.92 (t, J=5.2 Hz, 2H), 3.02 (t, J=5.6 Hz, 2H), 2.95-2.77 (m, 4H), 2.95-2.58 (m, 5H), 2.39-2.26 (m, 2H), 2.21-1.84 (m, 12H), 1.75-1.67 (m, 1H), 1.46-1.35 (m, 2H), 1.27-1.18 (m, 2H).

F NMR (400 MHz, DMSO-d₆) δ=−73.67, −94.95.

### Example 91. Preparation of Compound 169a

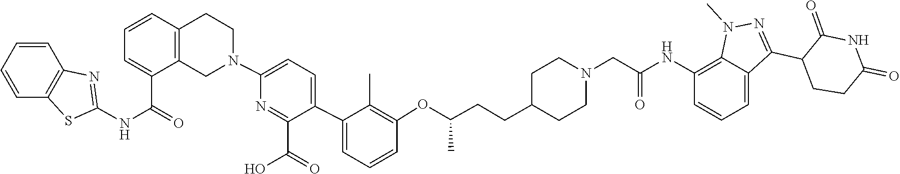 [SMILES: Cc1c(O[C@@H](C)CCC2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[(1S)-3-[1-[2-[[3-(2,6-dioxo-3-piperidyl)-1-methyl-indazol-7-yl]amino]-2-oxo-ethyl]-4-piperidyl]-1-methyl-propoxy]-2-methyl-phenyl]pyridine-2-carboxylic acid

### Step A. Procedure for Preparation of tert-butyl 6-[8-(1,3-benzothiazol-2-ylcarbamoyl)-3,4-dihydro-1H-isoquinolin-2-yl]-3-[3-[(1S)-3-[1-[2-[[3-(2,6-dioxo-3-piperidyl)-1-methyl-indazol-7-yl]amino]-2-oxo-ethyl]-4-piperidyl]-1-methyl-propoxy]-2-methyl-phenyl]pyridine-2-carboxylate

To a solution of 2-[4-[(3S)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]butyl]-1-piperidyl]acetic acid (100 mg, 126.59 μmol, 1 equiv.) and 3-(7-amino-1-methyl-indazol-3-yl)piperidine-2,6-dione (39.23 mg, 151.90 μmol, 1.2 equiv.) in DMF (0.5 mL) was added HATU (72.20 mg, 189.9 μmol, 1.5 equiv.) and DIEA (81.80 mg, 632.93 μmol, 110.24 μL, 5 equiv.). The mixture was stirred at 25° C. for 12 hours. The reaction mixture was quenched by addition water (10 mL), filtered, and concentrated under reduced pressure to give tert-butyl 6-[8-(1,3-benzothiazol-2-ylcarbamoyl)-3,4-dihydro-1H-isoquinolin-2-yl]-3-[3-[(1S)-3-[1-[2-[[3-(2,6-dioxo-3-piperidyl)-1-methyl-indazol-7-yl]amino]-2-oxo-ethyl]-4-piperidyl]-1-methyl-propoxy]-2-methyl-phenyl]pyridine-2-carboxylate (120 mg, 116.5 μmol, 92.0% yield) as a yellow solid.

MS (ESI) m/z: 1030.9 [M+H]⁺

### Step B. Procedure for Preparation of 6-[8-(1,3-benzothiazol-2-ylcarbamoyl)-3,4-dihydro-1H-isoquinolin-2-yl]-3-[3-[(1S)-3-[1-[2-[[3-(2,6-dioxo-3-piperidyl)-1-methyl-indazol-7-yl]amino]-2-oxo-ethyl]-4-piperidyl]-1-methyl-propoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[(1S)-3-[1-[2-[[3-(2,6-dioxo-3-piperidyl)-1-methyl-indazol-7-yl]amino]-2-oxo-ethyl]-4-piperidyl]-1-methyl-propoxy]-2-methyl-phenyl]pyridine-2-carboxylate (120 mg, 116.48 μmol, 1 equiv.) in DCM (1 mL) was added TFA (13.28 mg, 116.48 μmol, 8.62 μL, 1 equiv.). The mixture was stirred at 25° C. for 12 hours. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[(1S)-3-[1-[2-[[3-(2,6-dioxo-3-piperidyl)-1-methyl-indazol-7-yl]amino]-2-oxo-ethyl]-4-piperidyl]-1-methyl-propoxy]-2-methyl-phenyl]pyridine-2-carboxylic acid (20.7 mg, 19.5 μmol, 16.8% yield, 91.9% purity) as a white solid.

MS (ESI) m/z: 974.5 [M+H]⁺

¹H NMR (400 MHz, DMSO-d₆) δ=10.91 (s, 1H), 9.86 (s, 1H), 8.03 (d, J=8.0 Hz, 1H), 7.78 (d, J=8.0 Hz, 1H), 7.62 (d, J=7.2 Hz, 1H), 7.56 (d, J=8.4 Hz, 1H), 7.50-7.42 (m, 3H), 7.40-7.32 (m, 2H), 7.30 (d, J=7.2 Hz, 1H), 7.11-7.03 (m, 2H), 6.95 (d, J=8.8 Hz, 1H), 6.88 (d, J=8.4 Hz, 1H), 6.61 (d, J=7.2 Hz, 1H), 4.97 (s, 2H), 4.49-4.33 (m, 2H), 4.10 (s, 3H), 3.91 (t, J=5.6 Hz, 2H), 3.16 (br s, 2H), 3.02 (t, J=5.6 Hz, 2H), 2.93 (d, J=10.4 Hz, 2H), 2.69-2.59 (m, 2H), 2.39-2.31 (m, 1H), 2.23-2.11 (m, 3H), 1.88 (s, 3H), 1.74-1.56 (m, 4H), 1.38-1.18 (m, 8H).

### Example 92. Preparation of Compound 169b

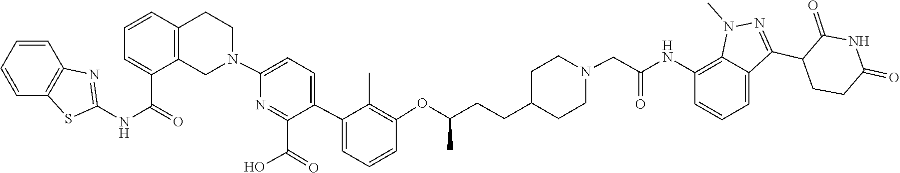 [SMILES: Cc1c(O[C@H](C)CCC2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[(1R)-3-[1-[2-[[3-(2,6-dioxo-3-piperidyl)-1-methyl-indazol-7-yl]amino]-2-oxo-ethyl]-4-piperidyl]-1-methyl-propoxy]-2-methyl-phenyl] pyridine-2-carboxylic acid

### Step A. Procedure for Preparation of tert-butyl 6-(8-(benzo[d]thiazol-2-ylcarbamoyl)-3,4-dihydroisoquinolin-2(1H)-yl)-3-(3-(((2R)-4-(1-(2-((3-(2,6-dioxopiperidin-3-yl)-1-methyl-11H-indazol-7-yl)amino)-2-oxoethyl)piperidin-4-yl)butan-2-yl)oxy)-2-methylphenyl)picolinate

To a solution of 2-[4-[(3R)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]butyl]-1-piperidyl]acetic acid (80 mg, 101.27 μmol, 1 equiv.) and 3-(7-amino-1-methyl-indazol-3-yl)piperidine-2,6-dione (28.77 mg, 111.40 μmol, 1.1 equiv.) in DMF (1 mL) was added HATU (57.76 mg, 151.91 μmol, 1.5 equiv.) and DIEA (39.26 mg, 303.81 μmol, 52.92 μL, 3 equiv.). The mixture was stirred at 25° C. for 2 hours. The mixture was quenched by water (2 mL) at 25° C., filtered, and concentrated under reduced pressure to give compound tert-butyl 6-(8-(benzo[d]thiazol-2-ylcarbamoyl)-3,4-dihydroisoquinolin-2(1H)-yl)-3-(3-(((2R)-4-(1-(2-((3-(2,6-dioxopiperidin-3-yl)-1-methyl-1H-indazol-7-yl)amino)-2-oxoethyl)piperidin-4-yl)butan-2-yl)oxy)-2-methylphenyl)picolinate (100 mg, 97.1 μmol, 95.9% yield) as a white solid.

MS (ESI) m/z: 1030.8[M+H]⁺

### Step B. Procedure for Preparation of 6-[8-(1,3-benzothiazol-2-ylcarbamoyl)-3,4-dihydro-1H-isoquinolin-2-yl]-3-[3-[(1R)-3-[1-[2-[[3-(2,6-dioxo-3-piperidyl)-1-methyl-indazol-7-yl]amino]-2-oxo-ethyl]-4-piperidyl]-1-methyl-propoxy]-2-methyl-phenyl] pyridine-2-carboxylic acid

A solution of tert-butyl 6-(8-(benzo[d]thiazol-2-ylcarbamoyl)-3,4-dihydroisoquinolin-2(1H)-yl)-3-(3-(((2R)-4-(1-(2-((3-(2,6-dioxopiperidin-3-yl)-1-methyl-1H-indazol-7-yl)amino)-2-oxoethyl)piperidin-4-yl)butan-2-yl)oxy)-2-methylphenyl)picolinate (100 mg, 97.06 μmol, 1 equiv.) in DCM (1 mL) and TFA (1 mL) was stirred at 25° C. for 2 hours. The reaction mixture was concentrated under reduced pressure to give a residue. The residue was purified by prep-HPLC to give compound 6-[8-(1,3-benzothiazol-2-ylcarbamoyl)-3,4-dihydro-1H-isoquinolin-2-yl]-3-[3-[(1R)-3-[1-[2-[[3-(2,6-dioxo-3-piperidyl)-1-methyl-indazol-7-yl]amino]-2-oxo-ethyl]-4-piperidyl]-1-methyl-propoxy]-2-methyl-phenyl] pyridine-2-carboxylic acid (31.9 mg, 32.3 μmol, 33.3% yield, 98.8% purity) as an off-white solid.

MS (ESI) m/z: 974.7 [M+H]⁺

¹H NMR (400 MHz, DMSO-d₆) δ=12.95-12.78 (m, 1H), 12.73-12.38 (m, 1H), 10.91 (s, 1H), 10.04-9.46 (m, 1H), 8.03 (d, J=8.0 Hz, 1H), 7.79 (d, J=8.0 Hz, 1H), 7.62 (d, J=6.8 Hz, 2H), 7.50-7.42 (m, 3H), 7.40-7.32 (m, 2H), 7.29-7.18 (m, 1H), 7.08 (t, J=8.0 Hz, 2H), 6.96 (d, J=8.4 Hz, 1H), 6.89 (d, J=8.0 Hz, 1H), 6.61 (d, J=7.6 Hz, 1H), 4.98 (s, 2H), 4.46-4.34 (m, 2H), 4.08 (s, 3H), 3.91 (t, J=5.6 Hz, 2H), 3.65-3.38 (m, 2H), 3.02 (t, J=5.6 Hz, 4H), 2.71-2.61 (m, 2H), 2.42-2.30 (m, 2H), 2.23-2.09 (m, 2H), 1.88 (s, 3H), 1.73-1.56 (m, 3H), 1.53-1.19 (m, 9H).

### Example 93. Preparation of Compound 170

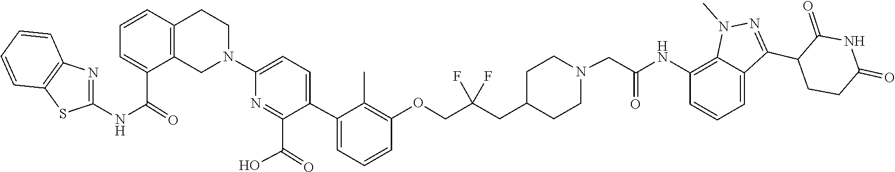 [SMILES: Cc1c(OCC(F)(F)CC2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-7-yl]amino]-2-oxo-ethyl]-4-piperidyl]-2,2-difluoro-propoxy]-2-methyl-phenyl]pyridine-2-carboxylic acid

### Step A. Procedure for Preparation of tert-butyl 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-7-yl]amino]-2-oxo-ethyl]-4-piperidyl]-2,2-difluoro-propoxy]-2-methyl-phenyl]pyridine-2-carboxylate

To a solution of 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-2,2-difluoro-propyl]-1-piperidyl]acetic acid (100 mg, 123.16 μmol, 1 equiv.) and 3-(7-amino-1-methyl-indazol-3-yl)piperidine-2,6-dione (38.17 mg, 147.80 μmol, 1.2 equiv.) in DMF (1 mL) was added HATU (56.20 mg, 147.80 μmol, 1.2 equiv.) and DIEA (47.75 mg, 369.49 μmol, 64.36 μL, 3 equiv.). The mixture was stirred at 25° C. for 1 hour. The reaction mixture was added into water (5 mL) and filtered to give tert-butyl 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-7-yl]amino]-2-oxo-ethyl]-4-piperidyl]-2,2-difluoro-propoxy]-2-methyl-phenyl]pyridine-2-carboxylate (110 mg, crude) as a yellow solid.

MS (ESI) m/z: 1052.5 [M+H]⁺

### Step B. Procedure for Preparation of 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-7-yl]amino]-2-oxo-ethyl]-4-piperidyl]-2,2-difluoro-propoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-7-yl]amino]-2-oxo-ethyl]-4-piperidyl]-2,2-difluoro-propoxy]-2-methyl-phenyl]pyridine-2-carboxylate (110 mg, 104.54 μmol, 1 equiv.) in DCM (1.1 mL) was added TFA (1.69 g, 14.86 mmol, 1.1 mL, 142.11 equiv.). The mixture was stirred at 25° C. for 12 hours. The mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-7-yl]amino]-2-oxo-ethyl]-4-piperidyl]-2,2-difluoro-propoxy]-2-methyl-phenyl]pyridine-2-carboxylic acid (35.5 mg, 35.4 μmol, 33.9% yield, 99.1% purity, FA) as an off-white solid.

MS (ESI) m/z: 996.6 [M+H]⁺

¹H NMR (400 MHz, DMSO-d₆) δ=13.27-11.99 (m, 1H), 10.90 (s, 1H), 9.85 (s, 1H), 8.03 (d, J=7.6 Hz, 1H), 7.79 (d, J=8.0 Hz, 1H), 7.62 (d, J=7.6 Hz, 1H), 7.56 (d, J=8.0 Hz, 1H), 7.50-7.42 (m, 3H), 7.40-7.32 (m, 2H), 7.29 (d, J=7.2 Hz, 1H), 7.16-7.10 (m, 1H), 7.07 (t, J=7.6 Hz, 1H), 7.00-6.92 (m, 2H), 6.71 (d, J=7.6 Hz, 1H), 4.98 (s, 2H), 4.41-4.34 (m, 1H), 4.29 (t, J=12 Hz, 2H), 4.10 (s, 3H), 3.92 (t, J=6.0 Hz, 2H), 3.15 (s, 2H), 3.02 (t, J=5.6 Hz, 2H), 2.95-2.88 (m, 2H), 2.71-2.57 (m, 2H), 2.39-2.32 (m, 1H), 2.26-2.12 (m, 3H), 2.09-1.97 (m, 2H), 1.92 (s, 3H), 1.80-1.65 (m, 3H), 1.50-1.37 (m, 2H)

### Example 94. Preparation of Compound 173b

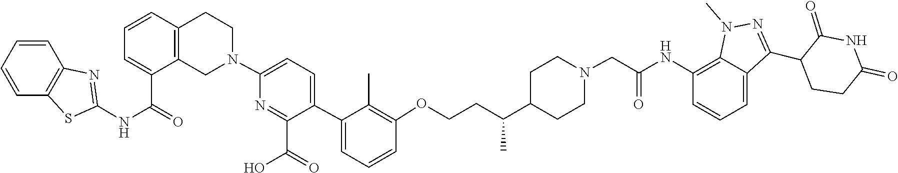 [SMILES: Cc1c(OCC[C@@H](C)C2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[(3R)-3-[1-[2-[[3-(2,6-dioxo-3-piperidyl)-1-methyl-indazol-7-yl]amino]-2-oxo-ethyl]-4-piperidyl]butoxy]-2-methyl-phenyl]pyridine-2-carboxylic acid

### Step A. Procedure for Preparation of tert-butyl 6-[8-(1,3-benzothiazol-2-ylcarbamoyl)-3,4-dihydro-1H-isoquinolin-2-yl]-3-[3-[(3R)-3-[1-[2-[[3-(2,6-dioxo-3-piperidyl)-1-methyl-indazol-7-yl]amino]-2-oxo-ethyl]-4-piperidyl]butoxy]-2-methyl-phenyl]pyridine-2-carboxylate

To a solution of 2-[4-[(1R)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-1-methyl-propyl]-1-piperidyl]acetic acid (100 mg, 126.59 μmol, 1 equiv.) and HATU (72.20 mg, 189.89 μmol, 1.5 equiv.) in DMF (1 mL) was added DIEA (49.08 mg, 379.77 μmol, 66.15 μL, 3 equiv.) and 3-(7-amino-1-methyl-indazol-3-yl)piperidine-2,6-dione (39.23 mg, 151.91 μmol, 1.2 equiv.). The mixture was stirred at 25° C. for 1 hour. The mixture was added into H₂O (5 mL) and filtered to give tert-butyl 6-[8-(1,3-benzothiazol-2-ylcarbamoyl)-3,4-dihydro-1H-isoquinolin-2-yl]-3-[3-[(3R)-3-[1-[2-[[3-(2,6-dioxo-3-piperidyl)-1-methyl-indazol-7-yl]amino]-2-oxo-ethyl]-4-piperidyl]butoxy]-2-methyl-phenyl]pyridine-2-carboxylate (105 mg, crude) as a yellow solid.

MS (ESI) m/z: 1030.8 [M+H]⁺

### Step B. Procedure for Preparation of 6-[8-(1,3-benzothiazol-2-ylcarbamoyl)-3,4-dihydro-1H-isoquinolin-2-yl]-3-[3-[(3R)-3-[1-[2-[[3-(2,6-dioxo-3-piperidyl)-1-methyl-indazol-7-yl]amino]-2-oxo-ethyl]-4-piperidyl]butoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[(3R)-3-[1-[2-[[3-(2,6-dioxo-3-piperidyl)-1-methyl-indazol-7-yl]amino]-2-oxo-ethyl]-4-piperidyl]butoxy]-2-methyl-phenyl]pyridine-2-carboxylate (105 mg, 101.92 μmol, 1 equiv.) in DCM (0.5 mL) was added TFA (770.00 mg, 6.75 mmol, 0.5 mL, 66.26 equiv.). The mixture was stirred at 25° C. for 12 hours. The mixture was concentrated under reduced pressure to remove DCM. The residue was purified by prep-HPLC to give 6-[8-(1,3-benzothiazol-2-ylcarbamoyl)-3,4-dihydro-1H-isoquinolin-2-yl]-3-[3-[(3R)-3-[1-[2-[[3-(2,6-dioxo-3-piperidyl)-1-methyl-indazol-7-yl]amino]-2-oxo-ethyl]-4-piperidyl]butoxy]-2-methyl-phenyl]pyridine-2-carboxylic acid (22.3 mg, 22.2 μmol, 21.8% yield, 96.9% purity) as a white solid.

MS (ESI) m/z: 974.6 [M+H]⁺

¹H NMR (400 MHz, DMSO-d₆) δ=13.37-11.91 (m, 1H), 10.90 (s, 1H), 9.87 (s, 1H), 8.02 (d, J=8.0 Hz, 1H), 7.78 (d, J=8.0 Hz, 1H), 7.62 (d, J=8.0 Hz, 1H), 7.56 (d, J=8.0 Hz, 1H), 7.49-7.40 (m, 3H), 7.40-7.29 (m, 3H), 7.08 (q, J=7.6 Hz, 2H), 6.96-6.87 (m, 2H), 6.63 (d, J=7.6 Hz, 1H), 4.98 (s, 2H), 4.40-4.34 (m, 1H), 4.11 (s, 3H), 4.06-3.95 (m, 2H), 3.91 (t, J=5.2 Hz, 2H), 3.15 (s, 2H), 3.04-2.97 (m, 4H), 2.70-2.60 (m, 2H), 2.41-2.29 (m, 2H), 2.18-2.13 (m, 2H), 1.89 (s, 3H), 1.66-1.52 (m, 4H), 1.49-1.38 (m, 2H), 1.32-1.15 (m, 2H), 0.92 (d, J=6.8 Hz, 3H)

### Example 95. Preparation of Compound 175

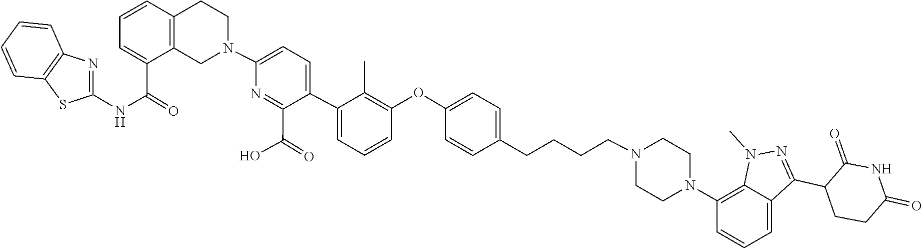 [SMILES: Cc1c(Oc2ccc(CCCCN3CCN(c4cccc5c(C6CCC(=O)NC6=O)nn(C)c45)CC3)cc2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(4-(4-(4-(3-(2,6-dioxopiperidin-3-yl)-1-methyl-1H-indazol-7-yl)piperazin-1-yl)butyl)phenoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(4-(4-(4-(3-(2,6-dioxopiperidin-3-yl)-1-methyl-1H-indazol-7-yl)piperazin-1-yl)butyl)phenoxy)-2-methylphenyl)picolinate

A mixture of tert-butyl 6-(8-(benzo[d]thiazol-2-ylcarbamoyl)-3,4-dihydroisoquinolin-2(1H)-yl)-3-(2-methyl-3-(4-(4-oxobutyl)phenoxy)phenyl)picolinate (75.00 mg, 101.50 μmol, 1 equiv.), 3-(1-methyl-7-piperazin-1-yl-indazol-3-yl)piperidine-2,6-dione (39.88 mg, 121.80 μmol, 1.2 equiv.), 4A MS (10 mg, 101.50 μmol, 1.00 equiv.) and HCOOH (97.52 ug, 2.03 μmol, 0.02 equiv.) in EtOH (2.4 mL) and THE (2.4 mL) was stirred at 25° C. for 2 h. NaBH₃CN (19.14 mg, 304.51 μmol, 3 equiv.) was added to the mixture, and the mixture was stirred at 25° C. for 14 h. The mixture was diluted with H₂O (2 mL×2) and extracted with ethyl acetate (2 mL×3). The combined organic layers were filtered and concentrated under reduced pressure to give tert-butyl 6-(8-(benzo[d]thiazol-2-ylcarbamoyl)-3,4-dihydroisoquinolin-2(1H)-yl)-3-(3-(4-(4-(4-(3-(2,6-dioxopiperidin-3-yl)-1-methyl-1H-indazol-7-yl)piperazin-1-yl)butyl)phenoxy)-2-methylphenyl)picolinate (100 mg, crude) as a yellow oil. It was used for next step without other purification.

MS (ESI) m/z:1050.6 [M+H]⁺.

### Step B. Procedure for Preparation of 6-(8-(benzo[d]thiazol-2-ylcarbamoyl)-3,4-dihydroisoquinolin-2(1H)-yl)-3-(3-(4-(4-(4-(3-(2,6-dioxopiperidin-3-yl)-1-methyl-1H-indazol-7-yl)piperazin-1-yl)butyl)phenoxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(3-(4-(4-(4-(3-(2,6-dioxopiperidin-3-yl)-1-methyl-1H-indazol-7-yl)piperazin-1-yl)butyl)phenoxy)-2-methylphenyl)picolinate (80 mg, 75.73 μmol, 1 equiv.), in TFA (0.8 mL) and DCM (0.8 mL) was stirred at 40° C. for 2 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(4-(4-(4-(3-(2,6-dioxopiperidin-3-yl)-1-methyl-1H-indazol-7-yl)piperazin-1-yl)butyl)phenoxy)-2-methylphenyl)picolinic acid (28.2 mg, 27.9 μmol, 36.9% yield, 98.3% purity) as a white solid.

MS (ESI) m/z: 994.8 [M+H]⁺.

¹H NMR (400 MHz, DMSO-d₆) δ=10.87 (s, 1H), 8.18 (s, 1H), 8.02 (d, J=7.2 Hz, 1H), 7.78 (d, J=8.0 Hz, 1H), 7.61 (d, J=7.2 Hz, 1H), 7.50-7.43 (m, 3H), 7.39-7.32 (m, 3H), 7.19-7.13 (m, 3H), 7.02-6.94 (m, 3H), 6.89 (d, J=7.2 Hz, 1H), 6.81 (d, J=8.0 Hz, 3H), 4.98 (s, 2H), 4.33 (dd, J=5.2, 9.2 Hz, 1H), 4.22 (s, 3H), 3.91 (t, J=5.6 Hz, 2H), 3.05-3.00 (m, 4H), 2.91-2.87 (m, 2H), 2.69-2.58 (m, 6H), 2.39 (t, J=7.2 Hz, 2H), 2.35-2.28 (m, 2H), 2.23-2.10 (m, 2H), 1.91 (s, 3H), 1.64-1.56 (m, 2H), 1.50-1.48 (m, 2H)

### Example 96. Preparation of Compound 191

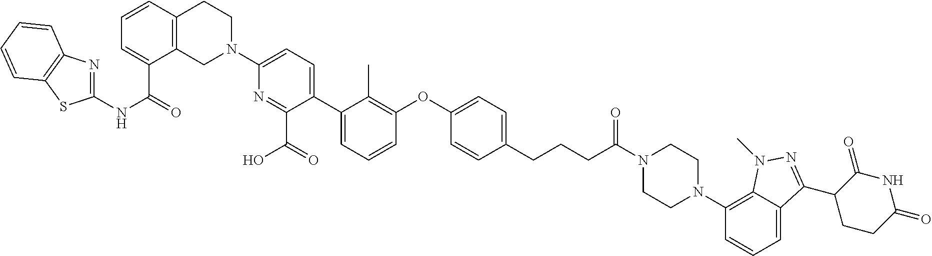 [SMILES: Cc1c(Oc2ccc(CCCC(=O)N3CCN(c4cccc5c(C6CCC(=O)NC6=O)nn(C)c45)CC3)cc2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(4-(4-(4-(3-(2,6-dioxopiperidin-3-yl)-1-methyl-1H-indazol-7-yl)piperazin-1-yl)-4-oxobutyl)phenoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(4-(4-(4-(3-(2,6-dioxopiperidin-3-yl)-1-methyl-1H-indazol-7-yl)piperazin-1-yl)-4-oxobutyl)phenoxy)-2-methylphenyl)picolinate

A mixture of 4-(4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)phenyl)butanoic acid (100 mg, 132.47 μmol, 1 equiv.), HATU (75.55 mg, 198.70 μmol, 1.5 equiv.), and DIEA (51.36 mg, 397.41 μmol, 69.22 μL, 3 equiv.) in DMAC (2 mL) was stirred at 25° C. for 0.25 h. 3-(1-Methyl-7-piperazin-1-yl-indazol-3-yl)piperidine-2,6-dione (52.04 mg, 158.96 μmol, 1.2 equiv.) was added to the mixture and stirred at 25° C. for 16 h. The mixture was diluted with H₂O (2 mL×2) and extracted with ethyl acetate (2 mL×3). The combined organic layers were filtered and concentrated under reduced pressure to give the crude compound tert-butyl 6-(8-(benzo[d]thiazol-2-ylcarbamoyl)-3,4-dihydroisoquinolin-2(1H)-yl)-3-(3-(4-(4-(4-(3-(2,6-dioxopiperidin-3-yl)-1-methyl-1H-indazol-7-yl)piperazin-1-yl)-4-oxobutyl)phenoxy)-2-methylphenyl)picolinate (100 mg, crude) as a yellow solid, it was used for next step without other purification.

MS (ESI) m/z: 1064.8 [M+H]⁺.

### Step B. Procedure for Preparation of 6-(8-(benzo[d]thiazol-2-ylcarbamoyl)-3,4-dihydroisoquinolin-2(1H)-yl)-3-(3-(4-(4-(4-(3-(2,6-dioxopiperidin-3-yl)-1-methyl-1H-indazol-7-yl)piperazin-1-yl)-4-oxobutyl)phenoxy)-2-methylphenyl)picolinic acid

A solution of tert-butyl 6-(8-(benzo[d]thiazol-2-ylcarbamoyl)-3,4-dihydroisoquinolin-2(1H)-yl)-3-(3-(4-(4-(4-(3-(2,6-dioxopiperidin-3-yl)-1-methyl-1H-indazol-7-yl)piperazin-1-yl)-4-oxobutyl)phenoxy)-2-methylphenyl)picolinate (100 mg, 93.96 μmol, 1 equiv.) in DCM (0.8 mL) and TFA (0.8 mL) was stirred at 40° C. for 2 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(4-(4-(4-(3-(2,6-dioxopiperidin-3-yl)-1-methyl-1H-indazol-7-yl)piperazin-1-yl)-4-oxobutyl)phenoxy)-2-methylphenyl)picolinic acid (33.1 mg, 30.5 μmol, 32.5% yield, 93.1% purity) as a white solid.

MS (ESI) m/z: 1008.8 [M+H]⁺.

¹H NMR (400 MHz, DMSO-d₆) δ=13.20-12.33 (m, 1H), 10.88 (s, 1H), 8.03 (d, J=7.6 Hz, 1H), 7.79 (d, J=8.0 Hz, 1H), 7.62 (d, J=7.6 Hz, 1H), 7.54-7.36 (m, 6H), 7.20-7.14 (m, 3H), 7.05-6.97 (m, 3H), 6.90-6.81 (m, 4H), 4.99 (s, 2H), 4.56-4.42 (m, 1H), 4.34 (dd, J=4.8, 9.6 Hz, 1H), 4.26 (s, 3H), 4.00-3.81 (m, 4H), 3.05-3.01 (m, 2H), 2.69-2.55 (m, 6H), 2.43-2.24 (m, 6H), 2.21-2.12 (m, 1H), 1.90 (s, 3H), 1.85-1.79 (m, 2H)

### Example 97. Preparation of Compound 196

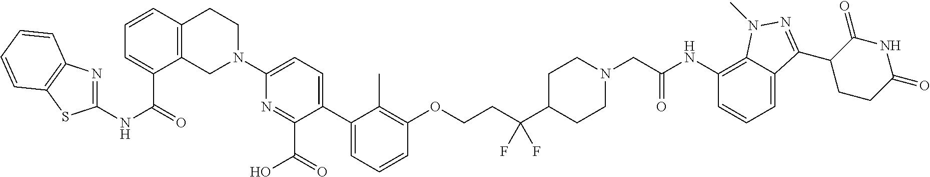 [SMILES: Cc1c(OCCC(F)(F)C2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-7-yl)amino)-2-oxoethyl)piperidin-4-yl)-3,3-difluoropropoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-7-yl)amino)-2-oxoethyl)piperidin-4-yl)-3,3-difluoropropoxy)-2-methylphenyl)picolinate

To a solution of 2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)-1,1-difluoropropyl)piperidin-1-yl)acetic acid (50.0 mg, 61.6 μmol, 1.0 equiv.) in DMF (0.50 mL) were added 3-(7-amino-1-methyl-1H-indazol-3-yl)piperidine-2,6-dione (17.5 mg, 67.7 μmol, 1.1 equiv.), HATU (28.1 mg, 73.9 μmol, 1.2 equiv.), and DIEA (23.9 mg, 185 μmol, 32.2 μL, 3.0 equiv.). The mixture was stirred at 20° C. for 1 hour. The reaction mixture was filtered and concentrated under reduced pressure. The residue was purified by prep-HPLC to give tert-butyl 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-7-yl)amino)-2-oxoethyl)piperidin-4-yl)-3,3-difluoropropoxy)-2-methylphenyl)picolinate (30.0 mg, 28.5 μmol, 46.3% yield) as a yellow solid.

MS (ESI) m/z: 1052.5 [M+H]⁺

### Step B. Procedure for Preparation of 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-7-yl)amino)-2-oxoethyl)piperidin-4-yl)-3,3-difluoropropoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-7-yl)amino)-2-oxoethyl)piperidin-4-yl)-3,3-difluoropropoxy)-2-methylphenyl)picolinate (30.0 mg, 28.5 μmol, 1.0 equiv.) in CH₂Cl₂ (1.00 mL) was added TFA (924 mg, 8.10 mmol, 600 μL, 284 equiv.). The mixture was stirred at 20° C. for 6 hours. After completion, the reaction mixture was concentrated under reduced pressure and the residue was purified by prep-HPLC to give 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-7-yl)amino)-2-oxoethyl)piperidin-4-yl)-3,3-difluoropropoxy)-2-methylphenyl)picolinic acid (10.64 mg, 10.6 μmol, 37.1% yield, 99.0% purity) as a yellow solid.

MS (ESI) m/z: 996.7 [M+H]⁺

¹H NMR (400 MHz, DMSO-d₆) δ=13.01-12.74 (m, 1H), 12.71-12.41 (m, 1H), 10.90 (s, 1H), 9.86 (s, 1H), 8.03 (d, J=7.6 Hz, 1H), 7.79 (d, J=7.6 Hz, 1H), 7.68-7.52 (m, 2H), 7.51-7.41 (m, 3H), 7.41-7.31 (m, 2H), 7.27 (d, J=7.2 Hz, 1H), 7.16-7.03 (m, 2H), 7.01-6.89 (m, 2H), 6.66 (d, J=7.6 Hz, 1H), 4.98 (s, 2H), 4.43-4.32 (m, 1H), 4.16 (t, J=5.6 Hz, 2H), 4.09 (s, 3H), 3.95-3.89 (m, 2H), 3.17 (s, 2H), 3.03 (t, J=5.6 Hz, 4H), 2.66-2.54 (m, 2H), 2.46-2.36 (m, 3H), 2.23-2.12 (m, 3H), 2.04-1.94 (m, 1H), 1.90 (s, 3H), 1.86-1.75 (m, 2H), 1.70-1.56 (m, 2H).

### Example 98. Preparation of Compound 199

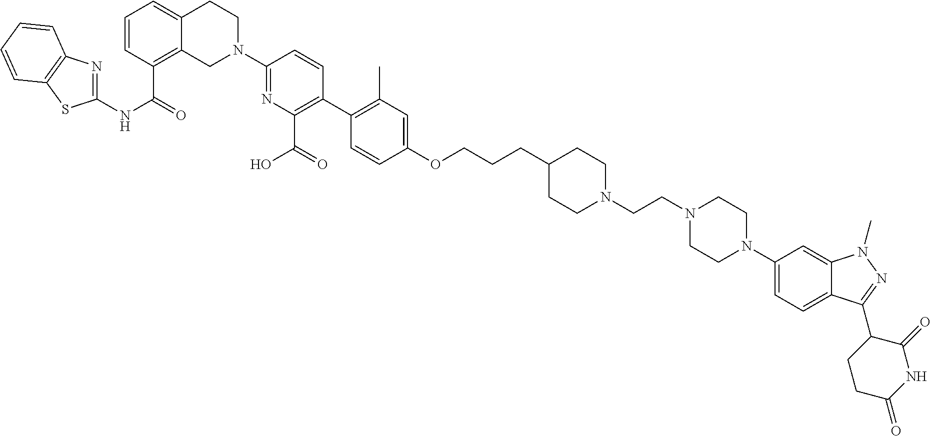 [SMILES: Cc1cc(OCCCC2CCN(CCN3CCN(c4ccc5c(C6CCC(=O)NC6=O)nn(C)c5c4)CC3)CC2)ccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[4-[3-[1-[2-[4-[3-(2,6-dioxo-3-piperidyl)-1-methyl-indazol-6-yl]piperazin-1-yl]ethyl]-4-piperidyl]propoxy]-2-methyl-phenyl]pyridine-2-carboxylic acid

### Step A. Procedure for Preparation of 6-[8-(1,3-benzothiazol-2-ylcarbamoyl)-3,4-dihydro-1H-isoquinolin-2-yl]-3-[4-[3-[1-[2-[4-[3-(2,6-dioxo-3-piperidyl)-1-methyl-indazol-6-yl]piperazin-1-yl]ethyl]-4-piperidyl]propoxy]-2-methyl-phenyl]pyridine-2-carboxylic acid

To a solution of 3-(1-methyl-6-piperazin-1-yl-indazol-3-yl)piperidine-2,6-dione (83.72 mg, 255.74 μmol, 0.9 equiv.) and NaBH(OAc)₃ (180.67 mg, 852.46 μmol, 3.0 equiv.) in IPA (2 mL) and DCM (2 mL) was added 6-[8-(1,3-benzothiazol-2-ylcarbamoyl)-3,4-dihydro-1H-isoquinolin-2-yl]-3-[2-methyl-4-[3-[1-(2-oxoethyl)-4-piperidyl]propoxy]phenyl]pyridine-2-carboxylic acid (200 mg, 284.15 μmol, 1.0 equiv.) at 0° C. slowly. The mixture was stirred at 0° C. for 1 hour, and then the mixture was warmed to 25° C. and stirred at 25° C. for 12 hours. The mixture was concentrated under reduced pressure to remove DCM and IPA. The residue was purified by prep-HPLC to give 6-[8-(1,3-benzothiazol-2-ylcarbamoyl)-3,4-dihydro-1H-isoquinolin-2-yl]-3-[4-[3-[1-[2-[4-[3-(2,6-dioxo-3-piperidyl)-1-methyl-indazol-6-yl]piperazin-1-yl]ethyl]-4-piperidyl]propoxy]-2-methyl-phenyl]pyridine-2-carboxylic acid (21.17 mg, 20.8 μmol, 7.3% yield, 99.8% purity) as a yellow solid.

MS (ESI) m/z: 1016.0 [M+H]⁺

¹H NMR (400 MHz, DMSO-d₆) δ=10.84 (s, 1H), 8.02 (d, J=7.6 Hz, 1H), 7.78 (d, J=8.0 Hz, 1H), 7.62 (d, J=7.2 Hz, 1H), 7.52-7.28 (m, 6H), 6.98-6.80 (m, 4H), 6.79-6.75 (m, 1H), 6.72-6.65 (m, 1H), 4.95 (s, 2H), 4.27-4.23 (m, 1H), 3.94-3.89 (m, 4H), 3.88 (s, 3H), 3.23-3.17 (m, 6H), 3.03-3.97 (m, 4H), 2.62-2.55 (m, 7H), 2.35-2.23 (m, 2H), 2.19-2.08 (m, 3H), 2.04 (s, 3H), 1.74-1.66 (m, 2H), 1.66-1.59 (m, 2H), 1.33-1.23 (m, 3H), 1.18-1.06 (m, 2H)

### Example 99. Preparation of Compound 202b

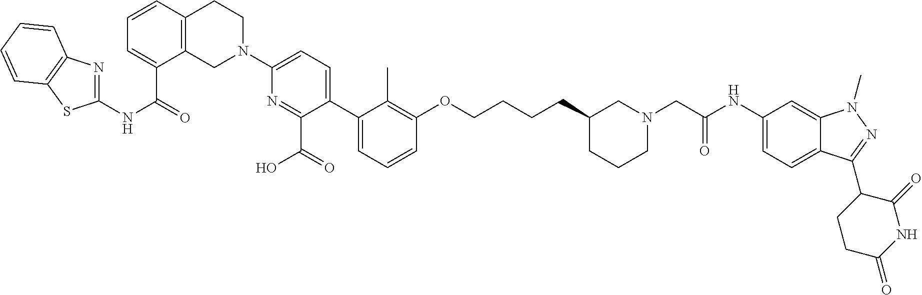 [SMILES: Cc1c(OCCCC[C@@H]2CCCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[4-[(3R)-1-[2-[[3-(2,6-dioxo-3-piperidyl)-1-methyl-indazol-6-yl]amino]-2-oxo-ethyl]-3-piperidyl]butoxy]-2-methyl-phenyl]pyridine-2-carboxylic acid

### Step A. Procedure for Preparation of tert-butyl 6-[8-(1,3-benzothiazol-2-ylcarbamoyl)-3,4-dihydro-1H-isoquinolin-2-yl]-3-[3-[4-[(3R)-1-[2-[[3-(2,6-dioxo-3-piperidyl)-1-methyl-indazol-6-yl]amino]-2-oxo-ethyl]-3-piperidyl]butoxy]-2-methyl-phenyl]pyridine-2-carboxylate

A mixture of 2-[(3R)-3-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]butyl]-1-piperidyl]acetic acid (91 mg, 115.19 μmol, 1 equiv.), 3-(6-amino-1-methyl-indazol-3-yl)piperidine-2,6-dione (35.70 mg, 138.23 μmol, 1.2 equiv.), HATU (52.56 mg, 138.23 μmol, 1.2 equiv.), and DIEA (44.66 mg, 345.58 μmol, 60.19 μL, 3 equiv.) in DMF (3 mL) was stirred at 25° C. for 12 hours. The reaction mixture was quenched by addition of water (3 mL), and then extracted with ethyl acetate (3 mL×3). The organic layers were combined and concentrated under reduced pressure to give tert-butyl 6-[8-(1,3-benzothiazol-2-ylcarbamoyl)-3,4-dihydro-1H-isoquinolin-2-yl]-3-[3-[4-[(3R)-1-[2-[[3-(2,6-dioxo-3-piperidyl)-1-methyl-indazol-6-yl]amino]-2-oxo-ethyl]-3-piperidyl]butoxy]-2-methyl-phenyl]pyridine-2-carboxylate (100 mg, crude) as a purple oil.

MS (ESI) m/z: 1030.6 [M+H]⁺.

### Step B. Procedure for Preparation of 6-[8-(1,3-benzothiazol-2-ylcarbamoyl)-3,4-dihydro-1H-isoquinolin-2-yl]-3-[3-[4-[(3R)-1-[2-[[3-(2,6-dioxo-3-piperidyl)-1-methyl-indazol-6-yl]amino]-2-oxo-ethyl]-3-piperidyl]butoxy]-2-methyl-phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[3-[4-[(3R)-1-[2-[[3-(2,6-dioxo-3-piperidyl)-1-methyl-indazol-6-yl]amino]-2-oxo-ethyl]-3-piperidyl]butoxy]-2-methyl-phenyl]pyridine-2-carboxylate (100 mg, 97.06 μmol, 1 equiv.) in TFA (1 mL) and DCM (1 mL) was stirred at 25° C. for 16 hours. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[4-[(3R)-1-[2-[[3-(2,6-dioxo-3-piperidyl)-1-methyl-indazol-6-yl]amino]-2-oxo-ethyl]-3-piperidyl]butoxy]-2-methyl-phenyl]pyridine-2-carboxylic acid (24.2 mg, 24.6 μmol, 25.3% yield, 98.8% purity) as a yellow solid.

MS (ESI) m/z: 974.9 [M+H]⁺.

¹H NMR (400 MHz, DMSO-d₆) δ=10.87 (s, 1H), 9.84 (s, 1H), 8.15 (s, 1H), 8.05-7.99 (m, 2H), 7.78 (d, J=8.0 Hz, 1H), 7.64-7.59 (m, 2H), 7.48-7.32 (m, 5H), 7.10-7.30 (m, 1H), 7.10-7.03 (m, 1H), 6.94 (d, J=8.8 Hz, 1H), 6.83 (d, J=8.0 Hz, 1H), 6.61 (d, J=7.6 Hz, 1H), 4.98 (s, 2H), 4.31 (dd, J=5.2, 9.6 Hz, 1H), 3.94-3.88 (m, 7H), 3.12 (d, J=11.2 Hz, 2H), 3.02 (t, J=5.6 Hz, 2H), 2.85-2.85 (m, 1H), 2.85-2.77 (m, 2H), 2.67-2.58 (m, 2H), 2.37-2.27 (m, 1H), 2.19-2.10 (m, 2H), 1.87 (s, 4H), 1.77-1.40 (m, 9H), 1.30-1.23 (m, 2H).

### Example 100. Preparation of Compound 210a

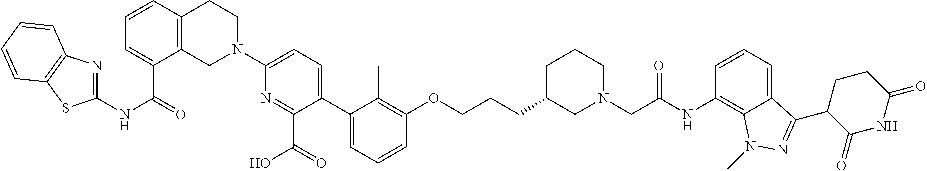 [SMILES: Cc1c(OCCC[C@@H]2CCCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-((3S)-1-(2-((3-(2,6-dioxopiperidin-3-yl)-1-methyl-1H-indazol-7-yl)amino)-2-oxoethyl)piperidin-3-yl)propoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(3-((3S)-1-(2-((3-(2,6-dioxopiperidin-3-yl)-1-methyl-1H-indazol-7-yl)amino)-2-oxoethyl)piperidin-3-yl)propoxy)-2-methylphenyl)picolinate

To a solution of (S)-2-(3-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)acetic acid (150 mg, 193.31 μmol, 1 equiv.) in DMF (1.5 mL) was added 3-(7-amino-1-methyl-1H-indazol-3-yl)piperidine-2,6-dione (49.93 mg, 193.31 μmol, 1 equiv.), HATU (80.85 mg, 212.64 mol, 1.1 equiv.) and DIPEA (74.95 mg, 579.93 μmol, 101.01 μL, 3 equiv.) at 25° C. The reaction mixture was stirred at 25° C. for 2 hours. The mixture was triturated with water (5 mL) and filtered to give tert-butyl 6-(8-(benzo[d]thiazol-2-ylcarbamoyl)-3,4-dihydroisoquinolin-2(1H)-yl)-3-(3-(3-((3S)-1-(2-((3-(2,6-dioxopiperidin-3-yl)-1-methyl-1H-indazol-7-yl)amino)-2-oxoethyl)piperidin-3-yl)propoxy)-2-methylphenyl)picolinate (132 mg, 105.2 μmol, 54.4% yield, 81% purity) as a yellow solid.

MS (ESI) m/z: 1016.7 [M+H]⁺.

### Step B. Procedure for Preparation of 6-(8-(benzo[d]thiazol-2-ylcarbamoyl)-3,4-dihydroisoquinolin-2(1H)-yl)-3-(3-(3-((3S)-1-(2-((3-(2,6-dioxopiperidin-3-yl)-1-methyl-11H-indazol-7-yl)amino)-2-oxoethyl)piperidin-3-yl)propoxy)-2-methylphenyl)picolinic acid

A solution of tert-butyl 6-(8-(benzo[d]thiazol-2-ylcarbamoyl)-3,4-dihydroisoquinolin-2(1H)-yl)-3-(3-(3-((3S)-1-(2-((3-(2,6-dioxopiperidin-3-yl)-1-methyl-1H-indazol-7-yl)amino)-2-oxoethyl)piperidin-3-yl)propoxy)-2-methylphenyl)picolinate (132 mg, 105.86 μmol, 81.5% purity, 1 equiv.) in DCM (1 mL) and TFA (1 mL) was stirred at 25° C. for 16 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(3-((3S)-1-(2-((3-(2,6-dioxopiperidin-3-yl)-1-methyl-1H-indazol-7-yl)amino)-2-oxoethyl)piperidin-3-yl)propoxy)-2-methylphenyl)picolinic acid (54.61 mg, 55.0 μmol, 51.9% yield, 96.7% purity) as a yellow solid.

MS (ESI) m/z: 960.6 [M+H]⁺.

¹H NMR (400 MHz, DMSO-d₆) δ=13.01-12.36 (m, 2H), 10.92 (s, 1H), 10.55 (s, 1H), 8.03 (d, J=8.0 Hz, 1H), 7.79 (d, J=8.0 Hz, 1H), 7.73-7.57 (m, 2H), 7.52-7.41 (m, 3H), 7.40-7.32 (m, 2H), 7.21 (d, J=7.2 Hz, 1H), 7.17-7.07 (m, 2H), 6.97 (d, J=8.8 Hz, 1H), 6.88 (d, J=8.4 Hz, 1H), 6.64 (d, J=7.6 Hz, 1H), 4.98 (s, 2H), 4.44-4.36 (m, 1H), 4.27 (s, 2H), 4.07 (s, 3H), 3.99-3.90 (m, 4H), 3.76 (s, 2H), 3.03 (t, J=5.6 Hz, 2H), 2.89-2.77 (m, 1H), 2.75-2.55 (m, 4H), 2.43-2.31 (m, 1H), 2.22-2.11 (m, 1H), 2.01-1.94 (m, 1H), 1.90 (s, 3H), 1.89-1.83 (m, 2H), 1.82-1.72 (m, 3H), 1.48-1.36 (m, 2H)

### Example 101. Preparation of Compound 212a

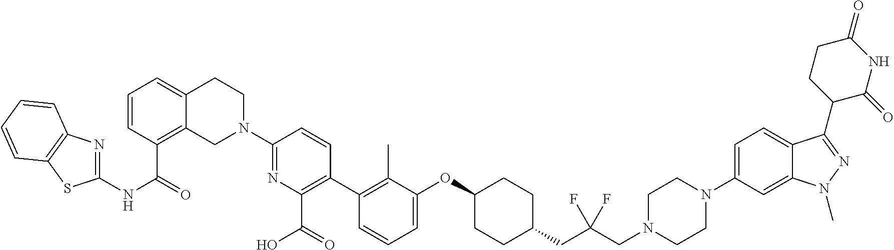 [SMILES: Cc1c(O[C@H]2CC[C@H](CC(F)(F)CN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-6-yl)piperazin-1-yl)-2,2-difluoropropyl)cyclohexyl)oxy)-2-methylphenyl)picolinic acid

### Step A. Procedure for Preparation of 3-[4-(3-bromo-2-methyl-phenoxy)cyclohexyl]-2,2-difluoro-propan-1-ol

A mixture of ethyl 3-((1r,4r)-4-(3-bromo-2-methylphenoxy)cyclohexyl)-2,2-difluoropropanoate (200 mg, 493.49 μmol, 1 equiv.), LiAlH₄ (15.92 mg, 419.47 μmol, 0.85 equiv.) in THE (2 mL) was stirred at 0° C. for 2 hours. The reaction mixture was quenched by addition Na₂SO₄·10H₂O (100 mg) under 0° C. and N₂ atmosphere, and then filtered and concentrated under reduced pressure to give a residue. The residue was purified by flash silica gel chromatography (Eluent of 0˜ 10% ethyl acetate/petroleum ether) to give 3-((1r,4r)-4-(3-bromo-2-methylphenoxy)cyclohexyl)-2,2-difluoropropan-1-ol (130 mg, 357.9 μmol, 72.5% yield) as a colorless oil.

¹H NMR (400 MHz, DMSO-d₆) δ=7.17-7.11 (m, 1H), 7.09-7.00 (m, 2H), 5.46 (s, 1H), 4.28-4.17 (m, 1H), 3.55 (t, J=13.6 Hz, 2H), 2.21 (s, 3H), 2.06-1.97 (m, 2H), 1.85-1.57 (m, 5H), 1.44-1.34 (m, 2H), 1.21-1.11 (m, 2H).

F NMR (400 MHz, DMSO-d₆) δ=−103.576

### Step B. Procedure for Preparation of 3-((1r,4r)-4-(3-bromo-2-methylphenoxy)cyclohexyl)-2,2-difluoropropyl trifluoromethanesulfonate

To a solution of 3-((1r,4r)-4-(3-bromo-2-methylphenoxy)cyclohexyl)-2,2-difluoropropan-1-ol (200 mg, 550.61 μmol, 1 equiv.) and pyridine (65.33 mg, 825.91 μmol, 66.66 μL, 1.5 equiv.) in CH₃CN (1.5 mL) was added trifluoromethylsulfonyl trifluoromethanesulfonate (170.88 mg, 605.67 μmol, 99.93 μL, 1.1 equiv.). The mixture was stirred at 0° C. for 1 hour. The reaction mixture was diluted with H₂O (2 mL) and extracted with DCM (10 mL). The combined organic layers were washed with 1N citric acid (2 mL) and 1N NaHCO₃ (2 mL), dried over Na₂SO₄, filtered, and concentrated under reduced pressure to give a residue. The residue was used for next step without other purification. The compound 3-((1r,4r)-4-(3-bromo-2-methylphenoxy)cyclohexyl)-2,2-difluoropropyl trifluoromethanesulfonate (270 mg, 545.13 μmol, 99.00% yield) was obtained as a colorless oil.

### Step C. Procedure for Preparation of 3-(6-(4-(3-((1r,4r)-4-(3-bromo-2-methylphenoxy)cyclohexyl)-2,2-difluoropropyl)piperazin-1-yl)-1-methyl-1H-indazol-3-yl)piperidine-2,6-dione

A mixture of 3-((1r,4r)-4-(3-bromo-2-methylphenoxy)cyclohexyl)-2,2-difluoropropyl trifluoromethanesulfonate (270 mg, 545.13 μmol, 1 equiv.), 3-(1-methyl-6-(piperazin-1-yl)-1H-indazol-3-yl)piperidine-2,6-dione (151.70 mg, 463.36 μmol, 0.85 equiv.), K₂CO₃ (188.35 mg, 1.36 mmol, 2.5 equiv.) in CH₃CN (1.5 mL) was stirred at 50° C. for 10 hours. The reaction mixture was filtered and concentrated under reduced pressure to give a residue. The residue was purified by flash silica gel chromatography (Eluent of 0˜20% ethyl acetate/petroleum ether) to give 3-(6-(4-(3-((1r,4r)-4-(3-bromo-2-methylphenoxy)cyclohexyl)-2,2-difluoropropyl)piperazin-1-yl)-1-methyl-1H-indazol-3-yl)piperidine-2,6-dione (120 mg, 178.4 μmol, 32.7% yield) as a white solid.

MS (ESI) m/z: 673.5 [M+H]⁺.

### Step D. Procedure for Preparation of tert-butyl 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-6-yl)piperazin-1-yl)-2,2-difluoropropyl)cyclohexyl)oxy)-2-methylphenyl)picolinate

A mixture of 3-(6-(4-(3-((1r,4r)-4-(3-bromo-2-methylphenoxy)cyclohexyl)-2,2-difluoropropyl)piperazin-1-yl)-1-methyl-1H-indazol-3-yl)piperidine-2,6-dione (153.00 mg, 249.78 μmol, 1.4 equiv.), 3-[6-[4-[3-[4-(3-bromo-2-methyl-phenoxy)cyclohexyl]-2,2-difluoro-propyl]piperazin-1-yl]-1-methyl-indazol-3-yl]piperidine-2,6-dione (120 mg, 178.41 μmol, 1 equiv.), [2-(2-aminophenyl)phenyl]palladium(1+); bis(1-adamantyl)-butyl-phosphane; methanesulfonate (25.99 mg, 35.68 μmol, 0.2 equiv.), and KF (1.5 M, 178.41 μL, 1.5 equiv.) in dioxane (0.4 mL) was degassed and purged with N₂ three times, and then the mixture was stirred at 100° C. for 1 hour under microwave. The reaction mixture was filtered and concentrated under reduced pressure to give a residue. The residue was purified by flash silica gel chromatography (Eluent of 0˜ 50% ethyl acetate/petroleum ether) to give tert-butyl 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-6-yl)piperazin-1-yl)-2,2-difluoropropyl)cyclohexyl)oxy)-2-methylphenyl)picolinate (100 mg, 92.7 μmol, 51.9% yield) as a pale yellow solid.

MS (ESI) m/z: 1080.4 [M+H]⁺.

### Step E. Procedure for Preparation of 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-6-yl)piperazin-1-yl)-2,2-difluoropropyl)cyclohexyl)oxy)-2-methylphenyl)picolinic acid

A solution of tert-butyl 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-6-yl)piperazin-1-yl)-2,2-difluoropropyl)cyclohexyl)oxy)-2-methylphenyl)picolinate (100 mg, 92.74 μmol, 1 equiv.) in TFA (0.5 mL) and DCM (0.5 mL) was stirred at 25° C. for 10 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-6-yl)piperazin-1-yl)-2,2-difluoropropyl)cyclohexyl)oxy)-2-methylphenyl)picolinic acid (37.0 mg, 35.5 μmol, 38.3% yield, 98.1% purity) as a white solid.

MS (ESI) m/z: 1022.6 [M+H]⁺.

¹H NMR (400 MHz, DMSO-d₆) δ=13.54-11.96 (m, 2H), 10.85 (s, 1H), 8.03 (d, J=7.6 Hz, 1H), 7.79 (d, J=8.0 Hz, 1H), 7.65-7.59 (m, 1H), 7.53-7.43 (m, 4H), 7.41-7.32 (m, 2H), 7.10-7.05 (m, 1H), 6.98-6.86 (m, 4H), 6.61 (d, J=7.2 Hz, 1H), 4.98 (s, 2H), 4.28-4.19 (m, 2H), 3.93-3.88 (m, 5H), 3.22 (s, 2H), 3.05-2.99 (m, 2H), 2.80-2.59 (m, 8H), 2.34-2.05 (m, 6H), 1.98-1.86 (m, 7H), 1.73-1.66 (m, 1H), 1.44-1.37 (m, 2H), 1.24-1.18 (m, 2H).

F NMR (400 MHz, DMSO-d₆) δ=−73.56, −94.83.

### Example 102. Preparation of Compound 216a

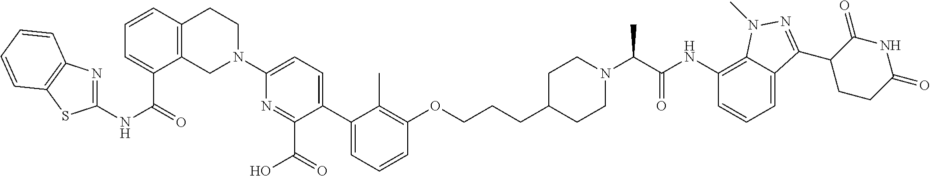 [SMILES: Cc1c(OCCCC2CCN([C@@H](C)C(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-(1-((2S)-1-((3-(2,6-dioxopiperidin-3-yl)-1-methyl-1H-indazol-7-yl)amino)-1-oxopropan-2-yl)piperidin-4-yl)propoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(3-(1-((2S)-1-((3-(2,6-dioxopiperidin-3-yl)-1-methyl-1H-indazol-7-yl)amino)-1-oxopropan-2-yl)piperidin-4-yl)propoxy)-2-methylphenyl)picolinate

To a solution of (S)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)propanoic acid (100 mg, 126.59 μmol, 1 equiv.), and 3-(7-amino-1-methyl-1H-indazol-3-yl)piperidine-2,6-dione (39.23 mg, 151.91 μmol, 1.2 equiv.) in pyridine (1 mL) was added EDCI (36.40 mg, 189.89 μmol, 1.5 equiv.). The mixture was stirred at 25° C. for 2 hours. The reaction mixture was quenched by addition of H₂O (2 mL). The reaction mixture was filtered, and the solid was diluted in DCM (10 mL). The mixture was dried over Na₂SO₄, filtered, and concentrated under reduced pressure to give tert-butyl 6-(8-(benzo[d]thiazol-2-ylcarbamoyl)-3,4-dihydroisoquinolin-2(1H)-yl)-3-(3-(3-(1-((2S)-1-((3-(2,6-dioxopiperidin-3-yl)-1-methyl-1H-indazol-7-yl)amino)-1-oxopropan-2-yl)piperidin-4-yl)propoxy)-2-methylphenyl)picolinate (100 mg, crude) as a yellow solid.

MS (ESI) m/z: 1030.8 [M+H]⁺

### Step B. Procedure for Preparation of 6-(8-(benzo[d]thiazol-2-ylcarbamoyl)-3,4-dihydroisoquinolin-2(1H)-yl)-3-(3-(3-(1-((2S)-1-((3-(2,6-dioxopiperidin-3-yl)-1-methyl-1H-indazol-7-yl)amino)-1-oxopropan-2-yl)piperidin-4-yl)propoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(3-(1-((2S)-1-((3-(2,6-dioxopiperidin-3-yl)-1-methyl-1H-indazol-7-yl)amino)-1-oxopropan-2-yl)piperidin-4-yl)propoxy)-2-methylphenyl)picolinate (100 mg, 87.33 μmol, 89.97% purity, 1 equiv.) in DCM (1 mL) was added TFA (1.54 g, 13.51 mmol, 1 mL, 154.66 equiv.). The mixture was stirred at 25° C. for 12 hours. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(3-(1-((2S)-1-((3-(2,6-dioxopiperidin-3-yl)-1-methyl-1H-indazol-7-yl)amino)-1-oxopropan-2-yl)piperidin-4-yl)propoxy)-2-methylphenyl)picolinic acid (51.4 mg, 52.5 μmol, 60.1% yield, 99.3% purity) as a white solid.

MS (ESI) m/z: 975.0 [M+H]⁺.

¹H NMR (400 MHz, DMSO-d₆) δ=13.16-12.54 (m, 1H), 10.90 (s, 1H), 10.05-9.73 (m, 1H), 8.14 (s, 1H), 8.03 (d, J=8.0 Hz, 1H), 7.79 (d, J=8.0 Hz, 1H), 7.66-7.54 (m, 2H), 7.50-7.41 (m, 3H), 7.41-7.32 (m, 2H), 7.25 (d, J=7.2 Hz, 1H), 7.08 (q, J=8.0 Hz, 2H), 6.96 (d, J=9.2 Hz, 1H), 6.91-6.84 (m, 1H), 6.63 (d, J=7.6 Hz, 1H), 4.98 (s, 2H), 4.39-4.35 (m, 1H), 4.10 (s, 3H), 3.97-3.89 (m, 4H), 3.02 (t, J=5.6 Hz, 2H), 2.98-2.85 (m, 2H), 2.70-2.59 (m, 2H), 2.44-2.30 (m, 2H), 2.28-2.11 (m, 2H), 1.89 (s, 3H), 1.83-1.65 (m, 4H), 1.50-1.09 (m, 9H).

### Example 103. Preparation of Compound 216b

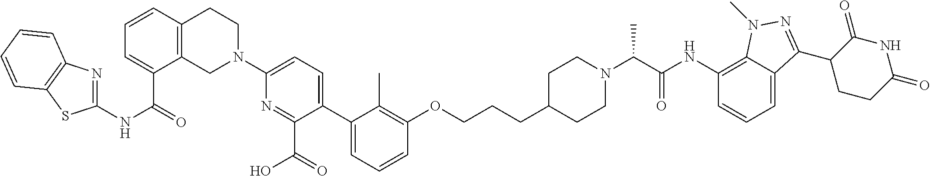 [SMILES: Cc1c(OCCCC2CCN([C@H](C)C(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-(1-((2R)-1-((3-(2,6-dioxopiperidin-3-yl)-1-methyl-1H-indazol-7-yl)amino)-1-oxopropan-2-yl)piperidin-4-yl)propoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(3-(1-((2R)-1-((3-(2,6-dioxopiperidin-3-yl)-1-methyl-11H-indazol-7-yl)amino)-1-oxopropan-2-yl)piperidin-4-yl)propoxy)-2-methylphenyl)picolinate

To a solution of (R)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)propanoic acid (100 mg, 112.93 μmol, 89.21% purity, 1 equiv.) and 3-(7-amino-1-methyl-1H-indazol-3-yl)piperidine-2,6-dione (35.00 mg, 135.51 μmol, 1.2 equiv.) in pyridine (1 mL) was added EDCI (32.47 mg, 169.39 μmol, 1.5 equiv.). The mixture was stirred at 25° C. for 3 hours. The reaction mixture was quenched by addition of H₂O (2 mL). The reaction mixture was filtered and the filter cake was washed with water (5 mL). The filter cake was then diluted in DCM (10 mL). The organic layers were dried over Na₂SO₄, filtered and concentrated under reduced pressure to give tert-butyl 6-(8-(benzo[d]thiazol-2-ylcarbamoyl)-3,4-dihydroisoquinolin-2(1H)-yl)-3-(3-(3-(1-((2R)-1-((3-(2,6-dioxopiperidin-3-yl)-1-methyl-1H-indazol-7-yl)amino)-1-oxopropan-2-yl)piperidin-4-yl)propoxy)-2-methylphenyl)picolinate (100 mg, crude) as a brown solid.

MS (ESI) m/z: 1030.8 [M+H]⁺

### Step B. Procedure for Preparation of 6-(8-(benzo[d]thiazol-2-ylcarbamoyl)-3,4-dihydroisoquinolin-2(1H)-yl)-3-(3-(3-(1-((2R)-1-((3-(2,6-dioxopiperidin-3-yl)-1-methyl-11H-indazol-7-yl)amino)-1-oxopropan-2-yl)piperidin-4-yl)propoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(3-(1-((2R)-1-((3-(2,6-dioxopiperidin-3-yl)-1-methyl-1H-indazol-7-yl)amino)-1-oxopropan-2-yl)piperidin-4-yl)propoxy)-2-methylphenyl)picolinate (100 mg, 97.06 μmol, 1 equiv.) in DCM (1 mL) was added TFA (1.54 g, 13.51 mmol, 1 mL, 139.14 equiv.). The mixture was stirred at 25° C. for 12 hours. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(3-(1-((2R)-1-((3-(2,6-dioxopiperidin-3-yl)-1-methyl-1H-indazol-7-yl)amino)-1-oxopropan-2-yl)piperidin-4-yl)propoxy)-2-methylphenyl)picolinic acid (55.0 mg, 55.0 μmol, 56.7% yield, 97.5% purity) as a white solid.

MS (ESI) m/z: 975.0 [M+H]⁺.

¹H NMR (400 MHz, DMSO-d₆) δ=10.89 (s, 1H), 9.86 (s, 1H), 8.15 (s, 1H), 8.03 (d, J=8.0 Hz, 1H), 7.79 (d, J=8.0 Hz, 1H), 7.62 (d, J=7.2 Hz, 1H), 7.56 (d, J=8.0 Hz, 1H), 7.51-7.41 (m, 3H), 7.40-7.31 (m, 2H), 7.25 (d, J=7.2 Hz, 1H), 7.08 (q, J=8.0 Hz, 2H), 6.96 (d, J=8.8 Hz, 1H), 6.87 (d, J=8.4 Hz, 1H), 6.63 (d, J=7.6 Hz, 1H), 4.98 (s, 2H), 4.38-4.34 (m, 1H), 4.10 (s, 3H), 3.97-3.89 (m, 4H), 3.02 (t, J=5.6 Hz, 2H), 2.91-2.84 (m, 2H), 2.68-2.60 (m, 2H), 2.39-2.30 (m, 2H), 2.24-2.12 (m, 2H), 1.89 (s, 3H), 1.81-1.68 (m, 4H), 1.41-1.40 (m, 2H), 1.35-1.17 (m, 7H).

### Example 104. Preparation of Compound 217

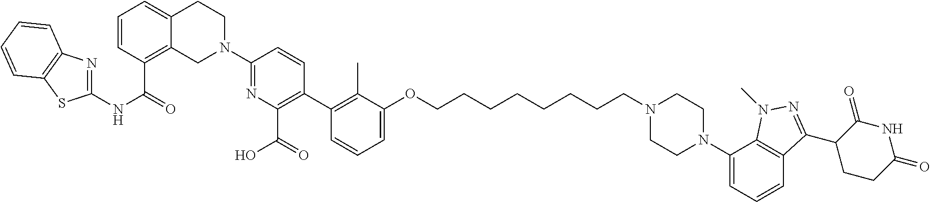 [SMILES: Cc1c(OCCCCCCCCN2CCN(c3cccc4c(C5CCC(=O)NC5=O)nn(C)c34)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[8-[4-[3-(2,6-dioxo-3-piperidyl)-1-methyl-indazol-7-yl]piperazin-1-yl]octoxy]-2-methyl-phenyl]pyridine-2-carboxylic acid

### Step A. Procedure for Preparation of tert-butyl 6-[8-(1,3-benzothiazol-2-ylcarbamoyl)-3,4-dihydro-1H-isoquinolin-2-yl]-3-[3-[8-[4-[3-(2,6-dioxo-3-piperidyl)-1-methyl-indazol-7-yl]piperazin-1-yl]octoxy]-2-methyl-phenyl]pyridine-2-carboxylate

A mixture of tert-butyl 6-[8-(1,3-benzothiazol-2-ylcarbamoyl)-3,4-dihydro-1H-isoquinolin-2-yl]-3-[2-methyl-3-(8-oxooctoxy)phenyl]pyridine-2-carboxylate (100 mg, 139.10 μmol, 1 equiv.), 3-(1-methyl-7-piperazin-1-yl-indazol-3-yl)piperidine-2,6-dione (54.65 mg, 166.92 μmol, 1.2 equiv.), NaBH(OAc)₃ (88.44 mg, 417.30 μmol, 3 equiv.), and AcOH (8.35 mg, 139.10 μmol, 7.96 μL, 1 equiv.) in DCM (2 mL) was degassed and purged with N₂ three times, and then the mixture was stirred at 25° C. for 3 hours under N₂ atmosphere. The reaction mixture was concentrated under reduced pressure to give a residue. The residue was used in the next step without purification. tert-Butyl 6-[8-(1,3-benzothiazol-2-ylcarbamoyl)-3,4-dihydro-1H-isoquinolin-2-yl]-3-[3-[8-[4-[3-(2,6-dioxo-3-piperidyl)-1-methyl-indazol-7-yl]piperazin-1-yl]octoxy]-2-methyl-phenyl]pyridine-2-carboxylate (100 mg, crude) was obtained as a white solid.

MS (ESI) m/z: 1030.8 [M+H]⁺

### Step B. Procedure for Preparation of 6-[8-(1,3-benzothiazol-2-ylcarbamoyl)-3,4-dihydro-1H-isoquinolin-2-yl]-3-[3-[8-[4-[3-(2,6-dioxo-3-piperidyl)-1-methyl-indazol-7-yl]piperazin-1-yl]octoxy]-2-methyl-phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[8-[4-[3-(2,6-dioxo-3-piperidyl)-1-methyl-indazol-7-yl]piperazin-1-yl]octoxy]-2-methyl-phenyl]pyridine-2-carboxylate (90.00 mg, 87.35 μmol, 1 equiv.) in TFA (1 mL) and DCM (1 mL). The mixture was stirred at 40° C. for 1 hour. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[8-[4-[3-(2,6-dioxo-3-piperidyl)-1-methyl-indazol-7-yl]piperazin-1-yl]octoxy]-2-methyl-phenyl]pyridine-2-carboxylic acid (21.5 mg, 20.4 μmol, 23.4% yield, 96.9% purity, FA) as a white solid.

MS (ESI) m/z: 975.1 [M+H]⁺

¹H NMR (400 MHz, DMSO-d₆) δ=12.90-12.82 (m, 1H), 10.89 (s, 1H), 8.14 (s, 1H), 8.01 (d, J=7.6 Hz, 1H), 7.77 (d, J=8.0 Hz, 1H), 7.62 (d, J=7.2 Hz, 1H), 7.42-7.39 (m, 7H), 7.11-7.01 (m, 3H), 6.96 (d, J=8.8 Hz, 1H), 6.88 (d, J=8.4 Hz, 1H), 6.63 (d, J=7.2 Hz, 1H), 5.00-4.95 (m, 2H), 4.36-4.33 (m, 1H), 4.25-4.22 (m, 3H), 3.99-3.89 (m, 5H), 3.08-2.96 (m, 6H), 2.92-2.85 (m, 3H), 2.70-2.56 (m, 3H), 2.34-2.30 (m, 1H), 2.18-2.14 (m, 1H), 1.90 (s, 3H), 1.78-1.72 (m, 2H), 1.64-1.55 (m, 2H), 1.49-1.44 (m, 2H), 1.41-1.25 (m, 7H).

### Example 105. Preparation of Compound 224a

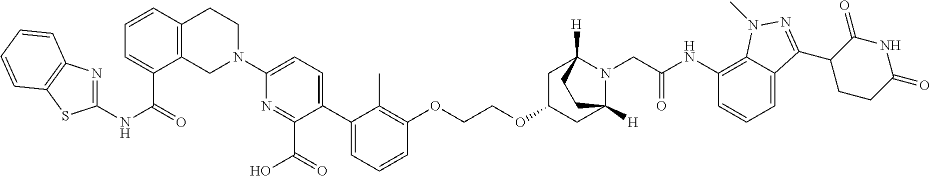 [SMILES: Cc1c(OCCO[C@@H]2C[C@H]3CC[C@@H](C2)N3CC(=O)Nc2cccc3c(C4CCC(=O)NC4=O)nn(C)c23)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(2-(((1R,3s,5S)-8-(2-((3-(2,6-dioxopiperidin-3-yl)-1-methyl-1H-indazol-7-yl)amino)-2-oxoethyl)-8-azabicyclo[3.2.1]octan-3-yl)oxy)ethoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(2-(((1R,3s,5S)-8-(2-((3-(2,6-dioxopiperidin-3-yl)-1-methyl-1H-indazol-7-yl)amino)-2-oxoethyl)-8-azabicyclo[3.2.1]octan-3-yl)oxy)ethoxy)-2-methylphenyl)picolinate

To a solution of 2-((1R,3s,5S)-3-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethoxy)-8-azabicyclo[3.2.1]octan-8-yl)acetic acid (110 mg, 136.82 μmol, 1 equiv.) in DMF (1 mL) was added HATU (62.43 mg, 164.19 μmol, 1.2 equiv.) and DIEA (53.05 mg, 410.47 μmol, 71.49 μL, 3 equiv.). Then 3-(7-amino-1-methyl-1H-indazol-3-yl)piperidine-2,6-dione (38.87 mg, 150.50 μmol, 1.1 equiv.) was added into the mixture and stirred at 25° C. for 12 hours. DCM 20 mL and water 20 mL were added and layers were separated. The aqueous was washed with DCM 20 mL (10 mL×2), dried over anhydrous sodium sulfate, filtered and concentrated under vacuum to give compound tert-butyl 6-(8-(benzo[d]thiazol-2-ylcarbamoyl)-3,4-dihydroisoquinolin-2(1H)-yl)-3-(3-(2-(((1R,3s,5S)-8-(2-((3-(2,6-dioxopiperidin-3-yl)-1-methyl-1H-indazol-7-yl)amino)-2-oxoethyl)-8-azabicyclo[3.2.1]octan-3-yl)oxy)ethoxy)-2-methylphenyl)picolinate (100 mg, 95.7 μmol, 69.9% yield) as a yellow solid.

MS (ESI) m/z: 1044.9 [M+H]⁺

### Step B. Procedure for Preparation of 6-(8-(benzo[d]thiazol-2-ylcarbamoyl)-3,4-dihydroisoquinolin-2(1H)-yl)-3-(3-(2-(((1R,3s,5S)-8-(2-((3-(2,6-dioxopiperidin-3-yl)-1-methyl-1H-indazol-7-yl)amino)-2-oxoethyl)-8-azabicyclo[3.2.1]octan-3-yl)oxy)ethoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(2-(((1R,3s,5S)-8-(2-((3-(2,6-dioxopiperidin-3-yl)-1-methyl-1H-indazol-7-yl)amino)-2-oxoethyl)-8-azabicyclo[3.2.1]octan-3-yl)oxy)ethoxy)-2-methylphenyl)picolinate (100 mg, 95.76 μmol, 1 equiv.) in DCM (2 mL) was added TFA (3.08 g, 27.01 mmol, 2 mL, 282.07 equiv.) and. The mixture was stirred at 25° C. for 12 hours. The reaction mixture was concentrated under reduced pressure to remove solvent. The residue was purified by prep-HPLC to give compound 6-(8-(benzo[d]thiazol-2-ylcarbamoyl)-3,4-dihydroisoquinolin-2(1H)-yl)-3-(3-(2-(((1R,3s,5S)-8-(2-((3-(2,6-dioxopiperidin-3-yl)-1-methyl-1H-indazol-7-yl)amino)-2-oxoethyl)-8-azabicyclo[3.2.1]octan-3-yl)oxy)ethoxy)-2-methylphenyl)picolinic acid (20.7 mg, 18.8 μmol, 19.7% yield, 94.0% purity, FA) as a yellow solid.

MS (ESI) m/z: 988.8 [M+H]⁺

¹H NMR (400 MHz, DMSO-d₆) δ=10.89 (s, 1H), 9.99 (s, 1H), 8.15 (s, 1H), 8.02 (d, J=7.6 Hz, 1H), 7.78 (d, J=8.0 Hz, 1H), 7.62 (d, J=7.2 Hz, 1H), 7.56 (d, J=8.0 Hz, 1H), 7.49-7.40 (m, 3H), 7.39-7.29 (m, 3H), 7.12-7.03 (m, 2H), 6.95 (d, J=8.8 Hz, 1H), 6.89 (d, J=8.0 Hz, 1H), 6.64 (d, J=7.6 Hz, 1H), 4.97 (s, 2H), 4.41-4.31 (m, 1H), 4.12 (s, 3H), 4.08-4.03 (m, 2H), 3.93-3.89 (m, 2H), 3.76-3.73 (m, 2H), 3.72-3.65 (m, 2H), 3.21 (s, 2H), 3.02 (t, J=5.2 Hz, 2H), 2.66 (d, J=4.0 Hz, 2H), 2.33 (d, J=1.6 Hz, 1H), 2.23-2.10 (m, 2H), 1.94-1.84 (m, 7H), 1.66 (t, J=10.8 Hz, 2H), 1.59 (d, J=7.6 Hz, 2H)

### Example 106. Preparation of Compound 231a

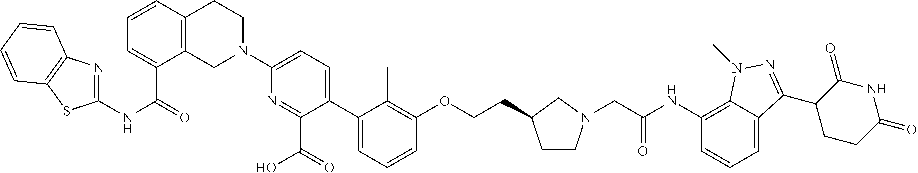 [SMILES: Cc1c(OCC[C@@H]2CCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[2-[(3S)-1-[2-[[3-(2,6-dioxo-3-piperidyl)-1-methyl-indazol-7-yl]amino]-2-oxo-ethyl]pyrrolidin-3-yl]ethoxy]-2-methyl-phenyl]pyridine-2-carboxylic acid

### Step A. Procedure for Preparation of tert-butyl 6-[8-(1,3-benzothiazol-2-ylcarbamoyl)-3,4-dihydro-1H-isoquinolin-2-yl]-3-[3-[2-[(3S)-1-[2-[[3-(2,6-dioxo-3-piperidyl)-1-methyl-indazol-7-yl]amino]-2-oxo-ethyl]pyrrolidin-3-yl]ethoxy]-2-methyl-phenyl]pyridine-2-carboxylate

A mixture of 2-[(3S)-3-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]ethyl]pyrrolidin-1-yl]acetic acid ((110 mg, 147.08 μmol, 1 equiv.), 3-(7-amino-1-methyl-indazol-3-yl)piperidine-2,6-dione (37.99 mg, 147.08 μmol, 1 equiv.), and EDCI (42.29 mg, 220.62 μmol, 1.5 equiv.) in pyridine (1 mL) was degassed and purged with N₂ three times, and then the mixture was stirred at 25° C. for 4 hours under N₂ atmosphere. The reaction mixture was diluted with water 5 mL and extracted with ethyl acetate 10 mL (5 mL×2). The combined organic layers were washed with brine (5 mL×2), dried over Na₂SO₄, filtered, and concentrated under reduced pressure to give a residue. The compound tert-butyl 6-[8-(1,3-benzothiazol-2-ylcarbamoyl)-3,4-dihydro-1H-isoquinolin-2-yl]-3-[3-[2-[(3S)-1-[2-[[3-(2,6-dioxo-3-piperidyl)-1-methyl-indazol-7-yl]amino]-2-oxo-ethyl]pyrrolidin-3-yl]ethoxy]-2-methyl-phenyl]pyridine-2-carboxylate (186 mg, crude) was obtained as a red oil.

### Step B. Procedure for Preparation of 6-[8-(1,3-benzothiazol-2-ylcarbamoyl)-3,4-dihydro-1H-isoquinolin-2-yl]-3-[3-[2-[(3S)-1-[2-[[3-(2,6-dioxo-3-piperidyl)-1-methyl-indazol-7-yl]amino]-2-oxo-ethyl]pyrrolidin-3-yl]ethoxy]-2-methyl-phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[3-[2-[(3S)-1-[2-[[3-(2,6-dioxo-3-piperidyl)-1-methyl-indazol-7-yl]amino]-2-oxo-ethyl]pyrrolidin-3-yl]ethoxy]-2-methyl-phenyl]pyridine-2-carboxylate (100 mg, 101.20 μmol, 1 equiv.) in TFA (1 mL) and DCM (1 mL) was degassed and purged with N₂ three times, and then the mixture was stirred at 25° C. for 24 hours under N₂ atmosphere. The reaction mixture was concentrated under reduced pressure to give a residue. The residue was purified by prep-HPLC. The compound 6-[8-(1,3-benzothiazol-2-ylcarbamoyl)-3,4-dihydro-1H-isoquinolin-2-yl]-3-[3-[2-[(3S)-1-[2-[[3-(2,6-dioxo-3-piperidyl)-1-methyl-indazol-7-yl]amino]-2-oxo-ethyl]pyrrolidin-3-yl]ethoxy]-2-methyl-phenyl]pyridine-2-carboxylic acid (48.5 mg, 51.3 μmol, 50.7% yield, 98.6% purity) was obtained as a yellow solid.

MS (ESI) m/z: 933.0 [M+H]⁺.

¹H NMR (400 MHz, DMSO-d₆) δ=12.89-12.83 (m, 1H), 12.67-12.45 (m, 1H), 10.91 (s, 1H), 10.45 (d, J=8.8 Hz, 1H), 10.29-10.07 (m, 1H), 8.02 (d, J=7.6 Hz, 1H), 7.78 (d, J=7.6 Hz, 1H), 7.71-7.56 (m, 2H), 7.50-7.30 (m, 5H), 7.25-7.06 (m, 3H), 7.01-6.87 (m, 2H), 6.65 (d, J=7.6 Hz, 1H), 4.98 (s, 2H), 4.43-4.37 (m, 2H), 4.05 (s, 5H), 3.91 (t, J=6.0 Hz, 2H), 3.71-3.62 (m, 1H), 3.03 (t, J=5.6 Hz, 2H), 2.74-2.60 (m, 3H), 2.43-2.26 (m, 6H), 2.23-2.09 (m, 2H), 1.99-1.85 (m, 5H)

### Example 107. Preparation of Compound 231b

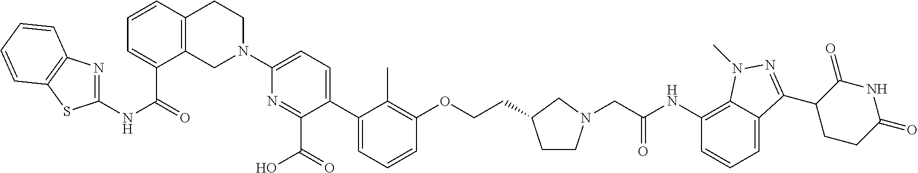 [SMILES: Cc1c(OCC[C@H]2CCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[2-[(3R)-1-[2-[[3-(2,6-dioxo-3-piperidyl)-1-methyl-indazol-7-yl]amino]-2-oxo-ethyl]pyrrolidin-3-yl]ethoxy]-2-methyl-phenyl]pyridine-2-carboxylic acid

### Step A. Procedure for Preparation of tert-butyl 6-[8-(1,3-benzothiazol-2-ylcarbamoyl)-3,4-dihydro-1H-isoquinolin-2-yl]-3-[3-[2-[(3R)-1-[2-[[3-(2,6-dioxo-3-piperidyl)-1-methyl-indazol-7-yl]amino]-2-oxo-ethyl]pyrrolidin-3-yl]ethoxy]-2-methyl-phenyl]pyridine-2-carboxylate

A mixture of 2-[(3R)-3-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]ethyl]pyrrolidin-1-yl]acetic acid (65 mg, 86.91 μmol, 1 equiv.), 3-(7-amino-1-methyl-indazol-3-yl)piperidine-2,6-dione (26.94 mg, 104.29 μmol, 1.2 eq), HATU (33.05 mg, 86.91 μmol, 1 equiv.), and DIPEA (33.70 mg, 260.73 μmol, 45.41 μL, 3 equiv.) in DMF (3 mL) was degassed and purged with N₂ three times, and then the mixture was stirred at 25° C. for 4 hours under N₂ atmosphere. The reaction mixture was added to water (3 mL), and then filtered. The mixture was concentrated to give a residue under reduced pressure. The residue was used in the next step without purification. The compound tert-butyl 6-[8-(1,3-benzothiazol-2-ylcarbamoyl)-3,4-dihydro-1H-isoquinolin-2-yl]-3-[3-[2-[(3R)-1-[2-[[3-(2,6-dioxo-3-piperidyl)-1-methyl-indazol-7-yl]amino]-2-oxo-ethyl]pyrrolidin-3-yl]ethoxy]-2-methyl-phenyl]pyridine-2-carboxylate (70 mg, crude) was obtained as a yellow solid.

MS (ESI) m/z: 988.8 [M+H]⁺

### Step B. Procedure for Preparation of 6-[8-(1,3-benzothiazol-2-ylcarbamoyl)-3,4-dihydro-1H-isoquinolin-2-yl]-3-[3-[2-[(3R)-1-[2-[[3-(2,6-dioxo-3-piperidyl)-1-methyl-indazol-7-yl]amino]-2-oxo-ethyl]pyrrolidin-3-yl]ethoxy]-2-methyl-phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[2-[(3R)-1-[2-[[3-(2,6-dioxo-3-piperidyl)-1-methyl-indazol-7-yl]amino]-2-oxo-ethyl]pyrrolidin-3-yl]ethoxy]-2-methyl-phenyl]pyridine-2-carboxylate (70 mg, 70.84 μmol, 1 equiv.) in TFA (2 mL) and DCM (2 mL) was stirred at 40° C. for 3 hours. The reaction mixture was concentrated under reduced pressure to remove DCM and TFA. The residue was purified by prep-HPLC. The compound 6-[8-(1,3-benzothiazol-2-ylcarbamoyl)-3,4-dihydro-1H-isoquinolin-2-yl]-3-[3-[2-[(3R)-1-[2-[[3-(2,6-dioxo-3-piperidyl)-1-methyl-indazol-7-yl]amino]-2-oxo-ethyl]pyrrolidin-3-yl]ethoxy]-2-methyl-phenyl]pyridine-2-carboxylic acid (24.2 mg, 25.8 μmol, 36.5% yield, 99.3% purity) was obtained as a yellow solid.

MS (ESI) m/z: 932.0 [M+H]⁺

¹H NMR (400 MHz, DMSO-d₆) δ=13.55-12.12 (m, 1H), 10.90 (s, 1H), 9.90 (s, 1H), 8.14 (s, 1H), 8.03 (d, J=7.6 Hz, 1H), 7.79 (d, J=8.0 Hz, 1H), 7.62 (d, J=7.6 Hz, 1H), 7.57 (d, J=8.0 Hz, 1H), 7.49-7.42 (m, 3H), 7.39-7.32 (m, 2H), 7.26 (d, J=7.2 Hz, 1H), 7.11-7.04 (m, 2H), 6.96 (d, J=8.8 Hz, 1H), 6.90 (d, J=8.0 Hz, 1H), 6.63 (d, J=7.2 Hz, 1H), 4.98 (s, 2H), 4.39-4.35 (m, 1H), 4.07 (s, 3H), 4.01-3.98 (m, 2H), 3.93-3.90 (m, 2H), 3.05-3.00 (m, 4H), 2.89-2.80 (m, 2H), 2.71-2.61 (m, 4H), 2.40-2.36 (m, 1H), 2.18-2.13 (m, 1H), 2.10-2.05 (m, 2H), 1.92-1.85 (m, 5H), 1.59-1.52 (m, 1H).

### Example 108. Preparation of Compound 237

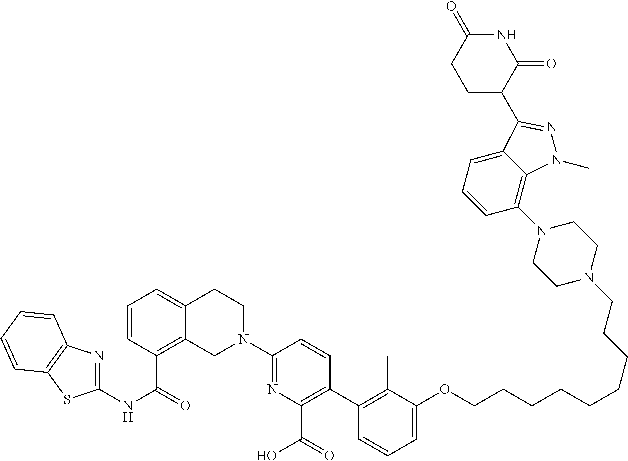 [SMILES: Cc1c(OCCCCCCCCCN2CCN(c3cccc4c(C5CCC(=O)NC5=O)nn(C)c34)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[9-[4-[3-(2,6-dioxo-3-piperidyl)-1-methyl-indazol-7-yl]piperazin-1-yl]nonoxy]-2-methyl-phenyl]pyridine-2-carboxylic acid

### Step A. Procedure for Preparation of tert-butyl 6-[8-(1,3-benzothiazol-2-ylcarbamoyl)-3,4-dihydro-1H-isoquinolin-2-yl]-3-[3-[9-[4-[3-(2,6-dioxo-3-piperidyl)-1-methyl-indazol-7-yl]piperazin-1-yl]nonoxy]-2-methyl-phenyl]pyridine-2-carboxylate

To a solution of 3-(1-methyl-7-piperazin-1-yl-indazol-3-yl)piperidine-2,6-dione (44.67 mg, 136.44 μmol, 1 equiv.) in DCM (5 mL) was added tert-butyl 6-[8-(1,3-benzothiazol-2-ylcarbamoyl)-3,4-dihydro-1H-isoquinolin-2-yl]-3-[2-methyl-3-(9-oxononoxy)phenyl]pyridine-2-carboxylate (100 mg, 136.44 μmol, 1 equiv.) and AcOH (8.19 mg, 136.44 μmol, 7.80 μL, 1 equiv.). The mixture was stirred at 25° C. for 1 hour, and then NaBH(OAc)₃ (28.92 mg, 136.44 μmol, 1 equiv.) was added. The mixture was stirred at 25° C. for 2 hours. The reaction mixture was concentrated under reduced pressure to remove solvent to give the crude product tert-butyl 6-[8-(1,3-benzothiazol-2-ylcarbamoyl)-3,4-dihydro-1H-isoquinolin-2-yl]-3-[3-[9-[4-[3-(2,6-dioxo-3-piperidyl)-1-methyl-indazol-7-yl]piperazin-1-yl]nonoxy]-2-methyl-phenyl]pyridine-2-carboxylate (100 mg, 95.7 μmol, 70.1% yield) as a yellow solid.

MS (ESI) m/z: 523.1 [M/2+H]⁺

### Step B. Procedure for Preparation of 6-[8-(1,3-benzothiazol-2-ylcarbamoyl)-3,4-dihydro-1H-isoquinolin-2-yl]-3-[3-[9-[4-[3-(2,6-dioxo-3-piperidyl)-1-methyl-indazol-7-yl]piperazin-1-yl]nonoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[9-[4-[3-(2,6-dioxo-3-piperidyl)-1-methyl-indazol-7-yl]piperazin-1-yl]nonoxy]-2-methyl-phenyl]pyridine-2-carboxylate (100 mg, 95.76 μmol, 1 equiv.) in TFA (1 mL) and DCM (1 mL). The mixture was stirred at 40° C. for 12 hours. The reaction mixture was concentrated under reduced pressure to give a residue. The residue was purified by reverse-phase HPLC to give 6-[8-(1,3-benzothiazol-2-ylcarbamoyl)-3,4-dihydro-1H-isoquinolin-2-yl]-3-[3-[9-[4-[3-(2,6-dioxo-3-piperidyl)-1-methyl-indazol-7-yl]piperazin-1-yl]nonoxy]-2-methyl-phenyl]pyridine-2-carboxylic acid (35.9 mg, 35.3 μmol, 36.9% yield, 97.1% purity) as a white solid.

MS (ESI) m/z: 988.7 [M+H]⁺.

¹H NMR (400 MHz, DMSO-d₆) δ=13.38-12.40 (m, 1H), 10.92 (s, 1H), 8.20 (s, 1H), 8.09 (d, J=8.0 Hz, 1H), 7.85 (d, J=8.0 Hz, 1H), 7.68 (d, J=7.6 Hz, 1H), 7.58-7.47 (m, 4H), 7.38 (s, 2H), 7.17-7.12 (m, 1H), 7.03-6.90 (m, 4H), 6.69 (d, J=7.6 Hz, 1H), 5.03 (s, 2H), 4.32 (dd, J=5.2, 9.2 Hz, 1H), 4.02 (t, J=6.0 Hz, 2H), 3.98-3.93 (m, 5H), 3.44-3.42 (m, 5H), 3.05 (t, J=5.6 Hz, 2H), 2.75-2.64 (m, 6H), 2.41-2.29 (m, 2H), 2.26-2.18 (m, 1H), 1.96 (s, 3H), 1.82-1.75 (m, 2H), 1.57-1.48 (m, 4H), 1.36 (s, 8H).

### Example 109. Preparation of Compound 242

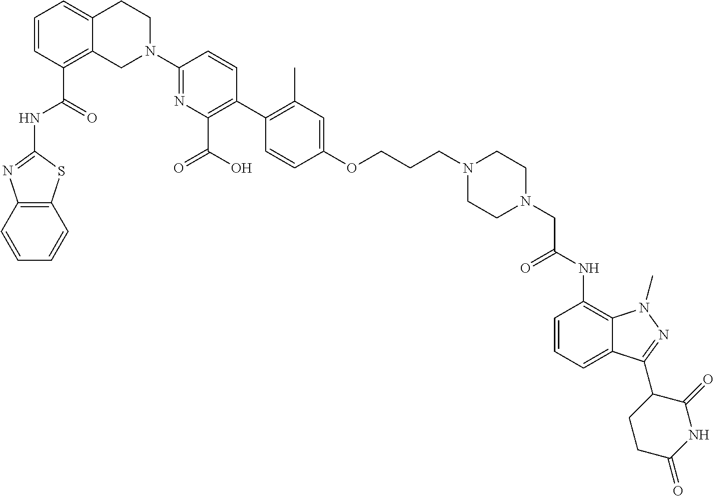 [SMILES: Cc1cc(OCCCN2CCN(CC(=O)Nc3cccc4c(C5CCC(=O)NC5=O)nn(C)c34)CC2)ccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[4-[3-[4-[2-[[3-(2,6-dioxo-3-piperidyl)-1-methyl-indazol-7-yl]amino]-2-oxo-ethyl]piperazin-1-yl]propoxy]-2-methyl-phenyl]pyridine-2-carboxylic acid

### Step A. Procedure for Preparation of tert-butyl 6-[8-(1,3-benzothiazol-2-ylcarbamoyl)-3,4-dihydro-1H-isoquinolin-2-yl]-3-[4-[3-[4-[2-[[3-(2,6-dioxo-3-piperidyl)-1-methyl-indazol-7-yl]amino]-2-oxo-ethyl]piperazin-1-yl]propoxy]-2-methyl-phenyl]pyridine-2-carboxylate

To a solution of 2-[4-[3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]propyl]piperazin-1-yl]acetic acid (80 mg, 102.97 μmol, 1 equiv.) in DMF (1 mL) was added HATU (78.30 mg, 205.94 μmol, 2 equiv.), DIPEA (39.92 mg, 308.90 μmol, 53.81 μL, 3 equiv.), and 3-(7-amino-1-methyl-indazol-3-yl)piperidine-2,6-dione (53.19 mg, 205.94 μmol, 2 equiv.). The mixture was stirred at 25° C. for 3 hours. The reaction mixture was quenched by addition of H₂O (2 mL). The reaction mixture was filtered and washed with water (5 mL). The mixture was then diluted with DCM (10 mL). The organic layers were dried over Na₂SO₄, filtered, and concentrated under reduced pressure to give tert-butyl 6-[8-(1,3-benzothiazol-2-ylcarbamoyl)-3,4-dihydro-1H-isoquinolin-2-yl]-3-[4-[3-[4-[2-[[3-(2,6-dioxo-3-piperidyl)-1-methyl-indazol-7-yl]amino]-2-oxo-ethyl]piperazin-1-yl]propoxy]-2-methyl-phenyl]pyridine-2-carboxylate (100 mg, crude) as a yellow solid.

MS (ESI) m/z: 1017.5 [M+H]⁺.

### Step B. Procedure for Preparation of 6-[8-(1,3-benzothiazol-2-ylcarbamoyl)-3,4-dihydro-1H-isoquinolin-2-yl]-3-[4-[3-[4-[2-[[3-(2,6-dioxo-3-piperidyl)-1-methyl-indazol-7-yl]amino]-2-oxo-ethyl]piperazin-1-yl]propoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[4-[3-[4-[2-[[3-(2,6-dioxo-3-piperidyl)-1-methyl-indazol-7-yl]amino]-2-oxo-ethyl]piperazin-1-yl]propoxy]-2-methyl-phenyl]pyridine-2-carboxylate (100 mg, 98.31 μmol, 1 equiv.) in DCM (0.5 mL) was added TFA (770.00 mg, 6.75 mmol, 500.00 μL, 68.69 equiv.). The mixture was stirred at 25° C. for 1 hour. The reaction mixture was concentrated under reduced pressure to give a residue. The crude product was purified by prep-HPLC to give 6-[8-(1,3-benzothiazol-2-ylcarbamoyl)-3,4-dihydro-1H-isoquinolin-2-yl]-3-[4-[3-[4-[2-[[3-(2,6-dioxo-3-piperidyl)-1-methyl-indazol-7-yl]amino]-2-oxo-ethyl]piperazin-1-yl]propoxy]-2-methyl-phenyl]pyridine-2-carboxylic acid (13.2 mg, 12.3 μmol, 12.5% yield, 89.4% purity) as a yellow solid.

MS (ESI) m/z: 961.7 [M+H]⁺

¹H NMR (400 MHz, DMSO-d₆) δ=10.88 (s, 1H), 9.83 (s, 1H), 8.14 (s, 1H), 8.03 (d, J=8.0 Hz, 1H), 7.79 (d, J=8.4 Hz, 1H), 7.62 (d, J=6.4 Hz, 1H), 7.57 (d, J=7.6 Hz, 1H), 7.51-7.41 (m, 3H), 7.40-7.29 (m, 3H), 7.08 (t, J=7.6 Hz, 1H), 6.98-6.89 (m, 2H), 6.79 (d, J=1.6 Hz, 1H), 6.73-6.69 (m, 1H), 4.97 (s, 2H), 4.32-4.33 (m, 1H), 4.12 (s, 2H), 4.07-3.95 (m, 3H), 3.91 (t, J=5.2 Hz, 2H), 3.64-3.56 (m, 2H), 3.20 (s, 3H), 3.03 (t, J=5.6 Hz, 4H), 2.96 (s, 1H), 2.64-2.58 (m, 4H), 2.25-2.13 (m, 3H), 2.07-1.99 (m, 4H), 1.91-1.83 (m, 2H).

### Example 110. Preparation of Compound 246a

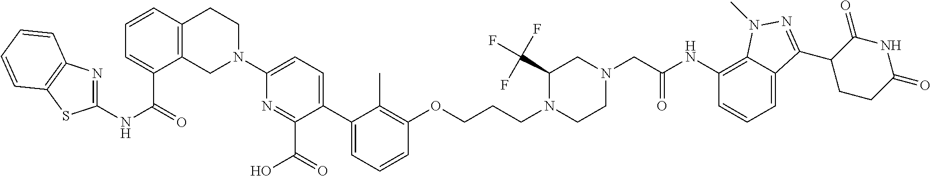 [SMILES: Cc1c(OCCCN2CCN(CC(=O)Nc3cccc4c(C5CCC(=O)NC5=O)nn(C)c34)C[C@@H]2C(F)(F)F)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-((2R)-4-(2-((3-(2,6-dioxopiperidin-3-yl)-1-methyl-1H-indazol-7-yl)amino)-2-oxoethyl)-2-(trifluoromethyl)piperazin-1-yl)propoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(3-((2R)-4-(2-((3-(2,6-dioxopiperidin-3-yl)-1-methyl-1H-indazol-7-yl)amino)-2-oxoethyl)-2-(trifluoromethyl)piperazin-1-yl)propoxy)-2-methylphenyl)picolinate

To a solution of (R)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-3-(trifluoromethyl)piperazin-1-yl)acetic acid (90 mg, 106.52 μmol, 1.0 equiv.) and 3-(7-amino-1-methyl-1H-indazol-3-yl)piperidine-2,6-dione (27.51 mg, 106.52 μmol, 1.0 equiv.) in pyridine (1.5 mL) was added EDCI (30.63 mg, 159.77 μmol, 1.5 equiv.). The mixture was stirred at 25° C. for 12 hours. The reaction mixture was diluted with water (2 mL) and extracted with ethyl acetate (2 mL×5). The combined organic layers were washed with brine (5 mL×2), dried over Na₂SO₄, filtered, and concentrated under reduced pressure to give tert-butyl 6-(8-(benzo[d]thiazol-2-ylcarbamoyl)-3,4-dihydroisoquinolin-2(1H)-yl)-3-(3-(3-((2R)-4-(2-((3-(2,6-dioxopiperidin-3-yl)-1-methyl-1H-indazol-7-yl)amino)-2-oxoethyl)-2-(trifluoromethyl)piperazin-1-yl)propoxy)-2-methylphenyl)picolinate (100 mg, crude) as a white solid.

MS (ESI) m/z: 1085.4 [M+H]⁺.

### Step B. Procedure for Preparation of 6-(8-(benzo[d]thiazol-2-ylcarbamoyl)-3,4-dihydroisoquinolin-2(1H)-yl)-3-(3-(3-((2R)-4-(2-((3-(2,6-dioxopiperidin-3-yl)-1-methyl-11H-indazol-7-yl)amino)-2-oxoethyl)-2-(trifluoromethyl)piperazin-1-yl)propoxy)-2-methylphenyl)picolinic acid

A solution of tert-butyl 6-(8-(benzo[d]thiazol-2-ylcarbamoyl)-3,4-dihydroisoquinolin-2(1H)-yl)-3-(3-(3-((2R)-4-(2-((3-(2,6-dioxopiperidin-3-yl)-1-methyl-1H-indazol-7-yl)amino)-2-oxoethyl)-2-(trifluoromethyl)piperazin-1-yl)propoxy)-2-methylphenyl)picolinate (100 mg, 92.15 μmol, 1.0 equiv.) in DCM (1 mL) was added TFA (770.00 mg, 6.75 mmol, 0.5 mL, 73.28 equiv.) was stirred at 25° C. for 12 hours. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(3-((2R)-4-(2-((3-(2,6-dioxopiperidin-3-yl)-1-methyl-1H-indazol-7-yl)amino)-2-oxoethyl)-2-(trifluoromethyl)piperazin-1-yl)propoxy)-2-methylphenyl)picolinic acid (72.0 mg, 69.5 μmol, 75.4% yield, 99.3% purity) as a white solid.

MS (ESI) m/z: 1029.4 [M+H]⁺.

¹H NMR (400 MHz, DMSO-d₆) δ=13.13-12.14 (m, 2H), 10.90 (s, 1H), 9.90-9.62 (m, 1H), 8.13 (s, 1H), 8.03 (d, J=7.6 Hz, 1H), 7.79 (d, J=8.0 Hz, 1H), 7.61 (t, J=6.4 Hz, 2H), 7.51-7.41 (m, 3H), 7.41-7.31 (m, 2H), 7.21 (d, J=7.2 Hz, 1H), 7.13-7.05 (m, 2H), 6.97 (d, J=8.8 Hz, 1H), 6.88 (d, J=8.4 Hz, 1H), 6.63 (d, J=7.6 Hz, 1H), 4.98 (s, 2H), 4.38 (dd, J=4.8, 5.2 Hz, 1H), 4.07 (s, 3H), 4.00 (t, J=5.6 Hz, 2H), 3.92 (t, J=5.6 Hz, 2H), 3.65-3.46 (m, 2H), 3.03 (t, J=5.6 Hz, 3H), 2.95-2.80 (m, 4H), 2.78-2.54 (m, 6H), 2.43-2.31 (m, 1H), 2.23-2.11 (m, 1H), 1.97-1.86 (m, 5H).

### Example 111. Preparation of Compound 255

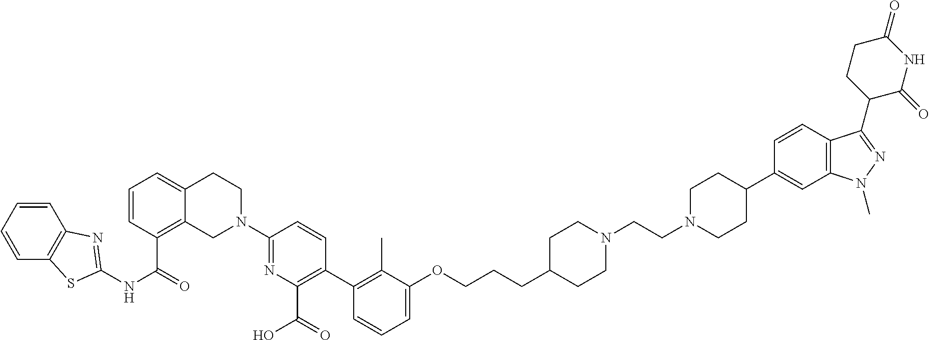 [SMILES: Cc1c(OCCCC2CCN(CCN3CCC(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[1-[2-[4-[3-(2,6-dioxo-3-piperidyl)-1-methyl-indazol-6-yl]-1-piperidyl]ethyl]-4-piperidyl]propoxy]-2-methyl-phenyl]pyridine-2-carboxylic acid

### Step A. Procedure for Preparation of tert-butyl 4-[3-(2,6-dibenzyloxy-3-pyridyl)-1-methyl-indazol-6-yl]-3,6-dihydro-2H-pyridine-1-carboxylate

A mixture of 6-bromo-3-(2,6-dibenzyloxy-3-pyridyl)-1-methyl-indazole (3.2 g, 6.40 mmol, 1.00 equiv.), tert-butyl 4-(4,4,5,5-tetramethyl-1,3,2-dioxaborolan-2-yl)-3,6-dihydro-2H-pyridine-1-carboxylate (2 g, 6.47 mmol, 1.01 equiv.), K₃PO₄ (4.07 g, 19.19 mmol, 3.00 equiv.), Ad₂nBuP Pd G₃ (465.73 mg, 639.51 μmol, 0.10 equiv.), and H₂O (3.20 g, 177.63 mmol, 3.20 mL, 27.78 equiv.) in dioxane (25 mL) was degassed and purged with N₂ three times, and then the mixture was stirred at 60° C. for 16 hours under N₂ atmosphere. The reaction mixture was concentrated under reduced pressure to give a residue. The residue was purified by flash silica gel chromatography (Eluent of 0˜20% ethyl acetate/petroleum ether) to give tert-butyl 4-[3-(2,6-dibenzyloxy-3-pyridyl)-1-methyl-indazol-6-yl]-3,6-dihydro-2H-pyridine-1-carboxylate (3.5 g, 5.8 mmol, 90.8% yield) as a white solid.

¹H NMR (400 MHz, DMSO-d₆) δ=7.92 (d, J=8.0 Hz, 1H), 7.64 (d, J=8.8 Hz, 1H), 7.59 (s, 1H), 7.51-7.45 (m, 2H), 7.42-7.28 (m, 8H), 7.17 (dd, J=1.2, 8.4 Hz, 1H), 6.59 (d, J=8.0 Hz, 1H), 6.26 (s, 1H), 5.46 (s, 2H), 5.43 (s, 2H), 4.08-4.06 (m, 3H), 4.05-4.03 (m, 2H), 3.59 (t, J=5.6 Hz, 2H), 2.62-2.55 (m, 2H), 1.45 (s, 9H)

### Step B. Procedure for Preparation of tert-butyl 4-[3-(2,6-dioxo-3-piperidyl)-1-methyl-indazol-6-yl]piperidine-1-carboxylate

A mixture of tert-butyl 4-[3-(2,6-dibenzyloxy-3-pyridyl)-1-methyl-indazol-6-yl]-3,6-dihydro-2H-pyridine-1-carboxylate (3.5 g, 5.81 mmol, 1.00 equiv.), Pd/C (3 g, 5.81 mmol, 10% purity, 1.00 equiv.), and EtOH (15 mL) in THE (15 mL) was degassed and purged with H₂ (15 Psi) three times, and then the mixture was stirred at 25° C. for 16 hours under H₂ (15 Psi) atmosphere. The reaction mixture was filtered and concentrated under reduced pressure to give crude tert-butyl 4-[3-(2,6-dioxo-3-piperidyl)-1-methyl-indazol-6-yl]piperidine-1-carboxylate (2.5 g, crude) as a black solid.

MS (ESI) m/z 427.3 [M+H]⁺.

### Step C. Procedure for Preparation of 3-[1-methyl-6-(4-piperidyl)indazol-3-yl]piperidine-2,6-dione

To a solution of tert-butyl 4-[3-(2,6-dioxo-3-piperidyl)-1-methyl-indazol-6-yl]piperidine-1-carboxylate (2.5 g, 5.86 mmol, 1.00 equiv.) in HCl/dioxane (20 mL) stirred at 25° C. for 1 hour. The reaction mixture was filtered to give crude 3-[1-methyl-6-(4-piperidyl)indazol-3-yl]piperidine-2,6-dione (1.5 g, 4.6 mmol, 78.4% yield) as a white solid.

### Step D. Procedure for Preparation of 6-[8-(1,3-benzothiazol-2-ylcarbamoyl)-3,4-dihydro-1H-isoquinolin-2-yl]-3-[3-[3-[1-[2-[4-[3-(2,6-dioxo-3-piperidyl)-1-methyl-indazol-6-yl]-1-piperidyl]ethyl]-4-piperidyl]propoxy]-2-methyl-phenyl]pyridine-2-carboxylic acid

A solution of 6-[8-(1,3-benzothiazol-2-ylcarbamoyl)-3,4-dihydro-1H-isoquinolin-2-yl]-3-[2-methyl-3-[3-[1-(2-oxoethyl)-4-piperidyl]propoxy]phenyl]pyridine-2-carboxylic acid (80 mg, 113.66 μmol, 1.00 equiv.) and 3-[1-methyl-6-(4-piperidyl)indazol-3-yl]piperidine-2,6-dione (37.10 mg, 113.66 μmol, 1.00 equiv.) in DCM (1 mL) and IPA (0.1 mL) was stirred at 25° C. for 15 hours. Then NaBH(OAc)₃ (72.27 mg, 340.98 μmol, 3 equiv.) was added into the solution. The mixture was stirred at 25° C. for 1 hour. The reaction mixture was filtered and concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[3-[1-[2-[4-[3-(2,6-dioxo-3-piperidyl)-1-methyl-indazol-6-yl]-1-piperidyl]ethyl]-4-piperidyl]propoxy]-2-methyl-phenyl]pyridine-2-carboxylic acid (14.0 mg, 11.9 μmol, 10.5% yield, 90.5% purity) as a yellow solid.

MS (ESI) m/z 1014.5 [M+H]⁺.

¹H NMR (400 MHz, DMSO-d₆) δ=10.87 (s, 1H), 8.23 (s, 2H), 8.01 (d, J=7.6 Hz, 1H), 7.77 (d, J=8.0 Hz, 1H), 7.64-7.56 (m, 2H), 7.49-7.43 (m, 1H), 7.42-7.38 (m, 2H), 7.37-7.30 (m, 3H), 7.09-6.99 (m, 2H), 6.89-6.81 (m, 2H), 6.66 (d, J=7.6 Hz, 1H), 4.94 (s, 2H), 4.31 (dd, J=5.2, 9.6 Hz, 1H), 4.00-3.95 (m, 2H), 3.95 (s, 3H), 3.87 (t, J=5.2 Hz, 2H), 3.04-2.94 (m, 8H), 2.63-2.59 (m, 2H), 2.18-2.03 (m, 6H), 1.90 (s, 3H), 1.82-1.65 (m, 8H), 1.62-1.51 (m, 2H), 1.40-1.24 (m, 4H), 1.16-0.99 (m, 2H)

### Example 112. Preparation of Compound 261

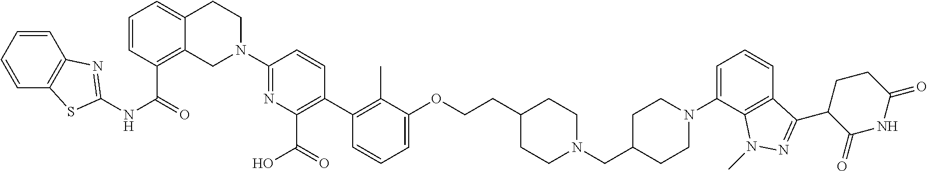 [SMILES: Cc1c(OCCC2CCN(CC3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[2-[1-[[1-[3-(2,6-dioxo-3-piperidyl)-1-methyl-indazol-7-yl]-4-piperidyl]methyl]-4-piperidyl]ethoxy]-2-methyl-phenyl]pyridine-2-carboxylic acid

### Step A. Procedure for Preparation of 6-[8-(1,3-benzothiazol-2-ylcarbamoyl)-3,4-dihydro-1H-isoquinolin-2-yl]-3-[3-[2-[1-[[1-[3-(2,6-dioxo-3-piperidyl)-1-methyl-indazol-7-yl]-4-piperidyl]methyl]-4-piperidyl]ethoxy]-2-methyl-phenyl]pyridine-2-carboxylic acid

To a solution of 6-[8-(1,3-benzothiazol-2-ylcarbamoyl)-3,4-dihydro-1H-isoquinolin-2-yl]-3-[2-methyl-3-[2-(4-piperidyl)ethoxy]phenyl]pyridine-2-carboxylic acid (130 mg, 189.99 μmol, 1.0 equiv., HCl) and 1-[3-(2,6-dioxo-3-piperidyl)-1-methyl-indazol-7-yl]piperidine-4-carbaldehyde (74.07 mg, 208.99 μmol, 1.1 equiv.) in DCM (1 mL) and IPA (0.1 mL) was stirred at 25° C. for 12 hours. Then NaBH(OAc)₃ (120.80 mg, 569.97 μmol, 3.0 equiv.) was added at 0° C. After addition, the mixture was stirred at 25° C. for 0.5 hour. The mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[2-[1-[[1-[3-(2,6-dioxo-3-piperidyl)-1-methyl-indazol-7-yl]-4-piperidyl]methyl]-4-piperidyl]ethoxy]-2-methyl-phenyl]pyridine-2-carboxylic acid (36.1 mg, 33.8 μmol, 17.8% yield, 96.5% purity) as a pink solid.

MS (ESI) m/z: 986.5 [M+H]⁺

¹H NMR (400 MHz, DMSO-d₆) δ=10.87 (s, 1H), 8.02 (d, J=7.6 Hz, 1H), 7.78 (d, J=8.0 Hz, 1H), 7.62 (d, J=7.2 Hz, 1H), 7.49-7.30 (m, 6H), 7.08 (t, J=8.0 Hz, 1H), 7.03-6.97 (m, 2H), 6.95-6.86 (m, 2H), 6.64 (d, J=7.6 Hz, 1H), 4.97 (s, 2H), 4.32 (dd, J=5.2, 9.6 Hz, 1H), 4.23 (s, 3H), 4.02-3.96 (m, 2H), 3.91 (t, J=5.6 Hz, 2H), 3.25-3.21 (m, 4H), 3.04-3.00 (m, 2H), 2.86 (d, J=9.6 Hz, 2H), 2.70-2.60 (m, 4H), 2.25-2.12 (m, 3H), 1.95-1.81 (m, 7H), 1.72-1.65 (m, 4H), 1.54-1.45 (m, 1H), 1.41-1.29 (m, 2H), 1.28-1.19 (m, 2H)

### Example 113. Preparation of Compound 24b

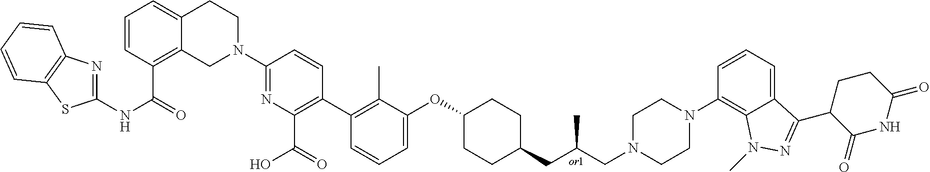 [SMILES: Cc1c(O[C@H]2CC[C@H](C[C@@H](C)CN3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1R,4r)-4-((2R)-3-(4-(3-(2,6-dioxopiperidin-3-yl)-1-methyl-1H-indazol-7-yl)piperazin-1-yl)-2-methylpropyl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1R,4r)-4-((2R*)-3-(4-(3-(2,6-dioxopiperidin-3-yl)-1-methyl-1H-indazol-7-yl)piperazin-1-yl)-2-methylpropyl)cyclohexyl)oxy)-2-methylphenyl)picolinic acid]

### Step A. Procedure for Preparation of 3-(7-(4-((R)-3-((1r,4R)-4-(3-bromo-2-methylphenoxy)cyclohexyl)-2-methylpropyl)piperazin-1-yl)-1-methyl-1H-indazol-3-yl)piperidine-2,6-dione

To a solution of 3-(1-methyl-7-(piperazin-1-yl)-1H-indazol-3-yl)piperidine-2,6-dione (128.69 mg, 353.70 μmol, 1.2 equiv., HCl) in DCM (2 mL) was added NaBH(OAc)₃ (187.41 mg, 884.26 μmol, 3 equiv.). Then (R)-3-((1r,4R)-4-(3-bromo-2-methylphenoxy)cyclohexyl)-2-methylpropanal (100 mg, 294.75 μmol, 1 equiv.) was added into the mixture and stirred at 0° C. for 1 hour. Then the mixture was stirred at 25° C. for 15 hours. DCM (30 mL) and water (30 mL) were added, and layers were separated. The aqueous phase was extracted with DCM (10 mL×2). The combined organic extracts were dried over anhydrous sodium sulfate, filtered, and concentrated under vacuum. The residue was purified by column chromatography (SiO₂, petroleum ether/ethyl acetate=4/1 to DCM:MeOH=10:1) to give 3-(7-(4-((R)-3-((1r,4R)-4-(3-bromo-2-methylphenoxy)cyclohexyl)-2-methylpropyl)piperazin-1-yl)-1-methyl-1H-indazol-3-yl)piperidine-2,6-dione (80 mg, 122.95 μmol, 41.71% yield) as a yellow solid.

MS (ESI) m/z: 652.1 [M+H]⁺(⁸⁰Br)

### Step B. Procedure for Preparation of tert-butyl 6-(8-(benzo[d]thiazol-2-ylcarbamoyl)-3,4-dihydroisoquinolin-2(1H)-yl)-3-(3-(((1R,4r)-4-((2R)-3-(4-(3-(2,6-dioxopiperidin-3-yl)-1-methyl-1H-indazol-7-yl)piperazin-1-yl)-2-methylpropyl)cyclohexyl)oxy)-2-methylphenyl)picolinate

A mixture of 3-(7-(4-((R)-3-((1r,4R)-4-(3-bromo-2-methylphenoxy)cyclohexyl)-2-methylpropyl)piperazin-1-yl)-1-methyl-1H-indazol-3-yl)piperidine-2,6-dione (80.00 mg, 122.95 μmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (90.38 mg, 147.55 μmol, 1.2 equiv.), KF (21.43 mg, 368.86 μmol, 8.64 μL, 3 equiv.), H₂O (0.1 mL), and [2-(2-aminophenyl)phenyl]palladium(1+); bis(1-adamantyl)-butyl-phosphane; methanesulfonate (8.95 mg, 12.30 μmol, 0.1 equiv.) in dioxane (1 mL) was degassed and purged with N₂ three times, and then the mixture was stirred at 100° C. for 1 hour under N₂ atmosphere. The reaction mixture was concentrated under reduced pressure to give a residue. The residue was purified by column chromatography (SiO₂, petroleum ether/ethyl acetate 3/1, DCM:MeOH=10:1) to give tert-butyl 6-(8-(benzo[d]thiazol-2-ylcarbamoyl)-3,4-dihydroisoquinolin-2(1H)-yl)-3-(3-(((1R,4r)-4-((2R)-3-(4-(3-(2,6-dioxopiperidin-3-yl)-1-methyl-1H-indazol-7-yl)piperazin-1-yl)-2-methylpropyl)cyclohexyl)oxy)-2-methylphenyl)picolinate (90 mg, 85.2 μmol, 69.3% yield) as a yellow solid.

MS (ESI) m/z: 1056.4 [M+H]⁺

### Step C. Procedure for Preparation of 6-(8-(benzo[d]thiazol-2-ylcarbamoyl)-3,4-dihydroisoquinolin-2(1H)-yl)-3-(3-(((1R,4r)-4-((2R)-3-(4-(3-(2,6-dioxopiperidin-3-yl)-1-methyl-1H-indazol-7-yl)piperazin-1-yl)-2-methylpropyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1R,4r)-4-((2R*)-3-(4-(3-(2,6-dioxopiperidin-3-yl)-1-methyl-1H-indazol-7-yl)piperazin-1-yl)-2-methylpropyl)cyclohexyl)oxy)-2-methylphenyl)picolinic acid]

To a solution of tert-butyl 6-(8-(benzo[d]thiazol-2-ylcarbamoyl)-3,4-dihydroisoquinolin-2(1H)-yl)-3-(3-(((1R,4r)-4-((2R)-3-(4-(3-(2,6-dioxopiperidin-3-yl)-1-methyl-1H-indazol-7-yl)piperazin-1-yl)-2-methylpropyl)cyclohexyl)oxy)-2-methylphenyl)picolinate (90 mg, 85.20 μmol, 1 equiv.) in DCM (1 mL) was added TFA (2.31 g, 20.26 mmol, 1.50 mL, 237.78 equiv.). The mixture was stirred at 25° C. for 12 hours. The reaction mixture was concentrated under reduced pressure to remove solvent. The residue was purified by prep-HPLC to give 6-(8-(benzo[d]thiazol-2-ylcarbamoyl)-3,4-dihydroisoquinolin-2(1H)-yl)-3-(3-(((1R,4r)-4-((2R)-3-(4-(3-(2,6-dioxopiperidin-3-yl)-1-methyl-1H-indazol-7-yl)piperazin-1-yl)-2-methylpropyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1R,4r)-4-((2R*)-3-(4-(3-(2,6-dioxopiperidin-3-yl)-1-methyl-1H-indazol-7-yl)piperazin-1-yl)-2-methylpropyl)cyclohexyl)oxy)-2-methylphenyl)picolinic acid] (40.4 mg, 39.5 μmol, 46.3% yield, 97.7% purity) as a white solid.

MS (ESI) m/z: 1000.5 [M+H]⁺.

¹H NMR (400 MHz, DMSO) δ=12.87 (s, 1H), 12.68-12.47 (m, 1H), 10.90 (s, 1H), 8.03 (d, J=8.0 Hz, 1H), 7.79 (d, J=8.0 Hz, 1H), 7.62 (d, J=8.4 Hz, 1H), 7.50-7.42 (m, 4H), 7.40-7.32 (m, 2H), 7.12-7.03 (m, 3H), 6.99-6.91 (m, 2H), 6.62 (d, J=7.6 Hz, 1H), 4.98 (s, 2H), 4.39-4.33 (m, 1H), 4.26 (s, 3H), 4.23-4.16 (m, 1H), 3.91 (t, J=6.0 Hz, 2H), 3.70-3.50 (m, 2H), 3.22-3.11 (m, 6H), 3.02 (t, J=5.6 Hz, 2H), 2.63-2.52 (m, 4H), 2.18 (d, J=5.6 Hz, 1H), 2.16-2.02 (m, 4H), 1.87 (s, 3H), 1.83-1.72 (m, 2H), 1.52-1.28 (m, 4H), 1.27-1.21 (m, 1H), 1.18-1.09 (m, 2H), 1.00 (d, J=6.0 Hz, 3H)

### Example 114. Preparation of Compound 287

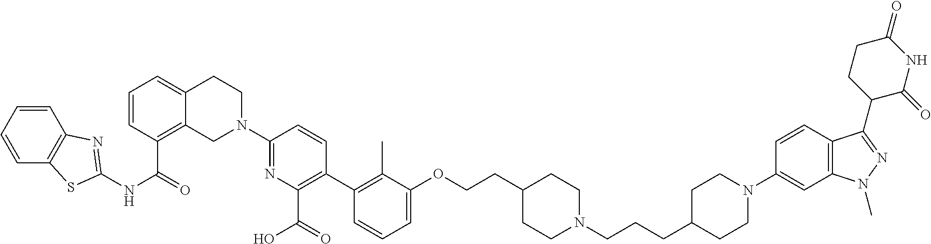 [SMILES: Cc1c(OCCC2CCN(CCCC3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[2-[1-[3-[1-[3-(2,6-dioxo-3-piperidyl)-1-methyl-indazol-6-yl]-4-piperidyl]propyl]-4-piperidyl]ethoxy]-2-methyl-phenyl]pyridine-2-carboxylic acid

### Step A. Procedure for Preparation of 3-(2,6-dibenzyloxy-3-pyridyl)-6-[4-(3,3-dimethoxypropyl)-1-piperidyl]-1-methyl-indazole

A mixture of 6-bromo-3-(2,6-dibenzyloxy-3-pyridyl)-1-methyl-indazole (200.00 mg, 399.69 μmol, 1.0 equiv.), 4-(3,3-dimethoxypropyl)piperidine (97.31 mg, 519.60 μmol, 1.3 equiv.), Cs₂CO₃ (390.68 mg, 1.20 mmol, 3.0 equiv.), and 1,3-bis[2,6-bis(1-propylbutyl)phenyl]-4,5-dichloro-2H-imidazol-1-ium-2-ide; 3-chloropyridine; dichloropalladium (38.88 mg, 39.97 μmol, 0.1 equiv.) in 2-methyl-2-butanol (2 mL) was degassed and purged with N₂ three times, and then the mixture was stirred at 100° C. for 16 hours under N₂ atmosphere. The mixture was filtered and concentrated under reduced pressure to give a residue. The residue was purified by flash silica gel chromatography (Eluent of 0-30% ethyl acetate/petroleum ether) to give 3-(2,6-dibenzyloxy-3-pyridyl)-6-[4-(3,3-dimethoxypropyl)-1-piperidyl]-1-methyl-indazole (150 mg, 236.6 μmol, 59.2% yield, 95.7% purity) as a green oil.

MS (ESI) m/z: 607.5 [M+H]⁺

¹H NMR (400 MHz, DMSO-d₆) δ=7.88 (d, J=8.0 Hz, 1H), 7.50-7.44 (m, 3H), 7.42-7.24 (m, 8H), 6.83-6.77 (m, 2H), 6.56 (d, J=8.0 Hz, 1H), 5.44 (s, 2H), 5.41 (s, 2H), 4.33 (t, J=5.6 Hz, 1H), 3.95 (s, 3H), 3.79-3.72 (m, 2H), 3.22 (s, 6H), 2.73-2.64 (m, 2H), 1.80-1.72 (m, 2H), 1.60-1.52 (m, 2H), 1.47-1.38 (m, 1H), 1.30-1.22 (m, 4H)

### Step B. Procedure for Preparation of 3-[6-[4-(3,3-dimethoxypropyl)-1-piperidyl]-1-methyl-indazol-3-yl]piperidine-2,6-dione

To a solution of 3-(2,6-dibenzyloxy-3-pyridyl)-6-[4-(3,3-dimethoxypropyl)-1-piperidyl]-1-methyl-indazole (150 mg, 247.22 μmol, 1.0 equiv.) in THE (2 mL) and EtOH (2 mL) was added Pd(OH)₂ (100 mg, 712.05 μmol, 2.88 equiv.) and Pd/C (100 mg, 10% purity) under N₂. The suspension was degassed under vacuum and purged with H₂ several times. The mixture was stirred under H₂ (15 psi) at 20° C. for 16 hours. The reaction mixture was filtered and concentrated under reduced pressure to give 3-[6-[4-(3,3-dimethoxypropyl)-1-piperidyl]-1-methyl-indazol-3-yl]piperidine-2,6-dione (90 mg, crude) as a yellow oil.

MS (ESI) m/z: 429.2 [M+H]⁺

¹H NMR (400 MHz, DMSO-d₆) δ=10.84 (s, 1H), 7.47 (d, J=9.2 Hz, 1H), 6.91-6.87 (m, 1H), 6.83-6.80 (m, 1H), 4.37-4.30 (m, 2H), 4.27-4.22 (m, 1H), 3.88 (s, 3H), 3.79-3.73 (m, 2H), 3.46-3.42 (m, 1H), 3.22 (s, 6H), 2.72-2.58 (m, 4H), 1.80-1.72 (m, 2H), 1.59-1.53 (m, 2H), 1.38 (s, 1H), 1.28-1.21 (m, 4H)

### Step C. Procedure for Preparation of 3-[1-[3-(2,6-dioxo-3-piperidyl)-1-methyl-indazol-6-yl]-4-piperidyl]propanal

A mixture of 3-[6-[4-(3,3-dimethoxypropyl)-1-piperidyl]-1-methyl-indazol-3-yl]piperidine-2,6-dione (90 mg, 210.02 μmol, 1.0 equiv.) and HCOOH (10.09 mg, 210.02 μmol, 1 mL, 1.0 equiv.) was stirred at 90° C. for 1 hour. The reaction mixture was concentrated under reduced pressure to give 3-[1-[3-(2,6-dioxo-3-piperidyl)-1-methyl-indazol-6-yl]-4-piperidyl]propanal (75 mg, crude) as a pink oil.

MS (ESI) m/z: 383.3 [M+H]⁺

### Step D. Procedure for Preparation of 6-[8-(1,3-benzothiazol-2-ylcarbamoyl)-3,4-dihydro-1H-isoquinolin-2-yl]-3-[3-[2-[1-[3-[1-[3-(2,6-dioxo-3-piperidyl)-1-methyl-indazol-6-yl]-4-piperidyl]propyl]-4-piperidyl]ethoxy]-2-methyl-phenyl]pyridine-2-carboxylic acid

To a solution of 6-[8-(1,3-benzothiazol-2-ylcarbamoyl)-3,4-dihydro-1H-isoquinolin-2-yl]-3-[2-methyl-3-[2-(4-piperidyl)ethoxy]phenyl]pyridine-2-carboxylic acid (100 mg, 146.15 μmol, 1.0 equiv., HCl) and 3-[1-[3-(2,6-dioxo-3-piperidyl)-1-methyl-indazol-6-yl]-4-piperidyl]propanal (72.66 mg, 189.99 μmol, 1.3 equiv.) in DCM (3 mL) and t-BuOH (1 mL) was stirred at 20° C. for 3 hours. NaBH(OAc)₃ (278.77 mg, 1.32 mmol, 9.0 equiv.) was added at 0° C. Then the mixture was warmed to 20° C. and stirred at 20° C. for 0.5 hour. The mixture was concentrated under reduced pressure to remove DCM and t-BuOH. The residue was purified by prep-HPLC to give 6-[8-(1,3-benzothiazol-2-ylcarbamoyl)-3,4-dihydro-1H-isoquinolin-2-yl]-3-[3-[2-[1-[3-[1-[3-(2,6-dioxo-3-piperidyl)-1-methyl-indazol-6-yl]-4-piperidyl]propyl]-4-piperidyl]ethoxy]-2-methyl-phenyl]pyridine-2-carboxylic acid (58.69 mg, 57.38 μmol, 39.26% yield, 99.16% purity) as a white solid.

MS (ESI) m/z: 1015.0 [M+H]⁺

¹H NMR (400 MHz, DMSO-d₆) δ=10.85 (s, 1H), 8.01 (d, J=7.6 Hz, 1H), 7.78 (d, J=8.0 Hz, 1H), 7.62 (d, J=6.4 Hz, 1H), 7.50-7.39 (m, 3H), 7.39-7.27 (m, 3H), 7.10-7.04 (m, 1H), 6.92-6.82 (m, 3H), 6.81 (s, 1H), 6.66 (d, J=7.6 Hz, 1H), 4.96 (s, 2H), 4.26-4.22 (m, 1H), 3.98-3.94 (m, 2H), 3.92-3.89 (m, 2H), 3.87 (s, 3H), 3.80-3.71 (m, 4H), 3.00 (t, J=5.6 Hz, 2H), 2.98-2.92 (m, 2H), 2.70-2.64 (m, 2H), 2.64-2.58 (m, 2H), 2.48-2.41 (m, 2H), 2.18-2.12 (m, 2H), 1.90 (s, 3H), 1.74 (d, J=11.6 Hz, 2H), 1.70-1.58 (m, 4H), 1.52 (s, 3H), 1.43-1.32 (m, 1H), 1.31-1.11 (m, 6H)

### Example 115. Preparation of Compound 288

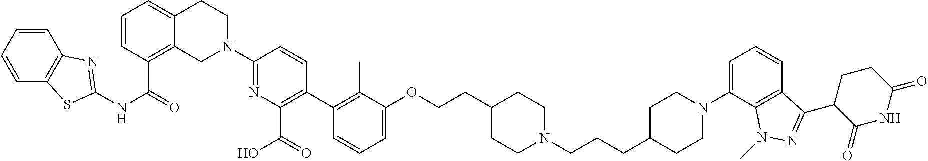 [SMILES: Cc1c(OCCC2CCN(CCCC3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[2-[1-[3-[1-[3-(2,6-dioxo-3-piperidyl)-1-methyl-indazol-7-yl]-4-piperidyl]propyl]-4-piperidyl]ethoxy]-2-methyl-phenyl]pyridine-2-carboxylic acid

### Step A. Procedure for Preparation of benzyl 4-(3-oxopropyl)piperidine-1-carboxylate

To a solution of (COCl)₂ (1.83 g, 14.42 mmol, 1.26 mL, 2.0 equiv.) in DCM (20 mL) was added DMSO (2.25 g, 28.84 mmol, 2.25 mL, 4.0 equiv.) dropwise slowly at −70° C. under N₂ atmosphere, and then the mixture was stirred at −70° C. for 1 hour. To the mixture was added benzyl 4-(3-hydroxypropyl)piperidine-1-carboxylate (2 g, 7.21 mmol, 1.0 equiv.) dropwise slowly at −70° C., and the mixture was stirred for 1 hour. Then TEA (4.38 g, 43.27 mmol, 6.02 mL, 6.0 equiv.) was added dropwise slowly at −70° C., and then the mixture was stirred at 20° C. for 0.5 hour under N₂ atmosphere. The reaction mixture was diluted with H₂O (50 mL) and extracted with DCM (50 mL×3). The combined organic layers were washed with H₂O (50 mL×3), dried over Na₂SO₄, filtered, and concentrated under reduced pressure to give a residue to benzyl 4-(3-oxopropyl)piperidine-1-carboxylate (1.97 g, crude) as a yellow oil.

¹H NMR (400 MHz, DMSO-d₆) δ=9.69-9.63 (m, 1H), 7.48-7.20 (m, 5H), 5.05 (s, 2H), 4.04-3.93 (m, 2H), 2.83-2.66 (m, 2H), 2.48-2.42 (m, 2H), 1.68-1.57 (m, 2H), 1.50-1.42 (m, 2H), 1.42-1.33 (m, 1H), 1.04-0.91 (m, 2H)

### Step B. Procedure for Preparation of benzyl 4-(3,3-dimethoxypropyl)piperidine-1-carboxylate

To a solution of benzyl 4-(3-oxopropyl)piperidine-1-carboxylate (1.97 g, 7.15 mmol, 1 equiv.) in MeOH (20 mL) was added TsOH (1.48 g, 8.59 mmol, 1.2 equiv.) and 4A MS (3 g, 7.15 mmol, 1.0 equiv.). The mixture was stirred at 80° C. for 24 hours. The mixture was filtered to give a filtrate, which was treated with saturated NaHCO₃ solution to pH=8 and concentrated under reduced pressure to give a residue. The residue was diluted with H₂O (50 mL) and extracted with DCM (50 mL×3). The combined organic layers were dried over Na₂SO₄, filtered, and concentrated under reduced pressure to give benzyl 4-(3,3-dimethoxypropyl)piperidine-1-carboxylate (1.9 g, crude) as a yellow oil.

¹H NMR (400 MHz, DMSO-d₆) δ=7.40-7.28 (m, 5H), 5.05 (s, 2H), 4.30 (t, J=5.6 Hz, 1H), 4.01-3.94 (m, 2H), 3.20 (s, 6H), 2.84-2.66 (m, 2H), 1.67-1.60 (m, 2H), 1.54-1.47 (m, 2H), 1.43-1.33 (m, 1H), 1.23-1.16 (m, 2H), 1.02-0.91 (m, 2H)

### Step C. Procedure for Preparation of 4-(3,3-dimethoxypropyl)piperidine

To a solution of benzyl 4-(3,3-dimethoxypropyl)piperidine-1-carboxylate (1.9 g, 5.91 mmol, 1.0 equiv.) in THF (50 mL) was added Pd/C (500 mg, 10% purity) under N₂. The suspension was degassed under vacuum and purged with H₂ several times. The mixture was stirred under H₂ (50 psi) at 50° C. for 16 hours. The reaction mixture was filtered, and the filtrate was concentrated under reduced pressure to give a residue. The residue was purified by flash silica gel chromatography (Eluent of 0˜100% Methanol/Dichloromethane) to give 4-(3,3-dimethoxypropyl)piperidine (655 mg, 3.1 mmol, 53.2% yield, 90.0% purity) as a yellow oil.

¹H NMR (400 MHz, DMSO-d₆) δ=4.29 (t, J=5.6 Hz, 1H), 3.19 (s, 6H), 2.97-2.88 (m, 2H), 2.8-2.40 (m, 2H), 1.61-1.53 (m, 2H), 1.53-1.46 (m, 2H), 1.30-1.20 (m, 1H), 1.20-1.13 (m, 2H), 1.04-0.92 (m, 2H)

### Step D. Procedure for Preparation of 3-(2,6-dibenzyloxy-3-pyridyl)-7-[4-(3,3-dimethoxypropyl)-1-piperidyl]-1-methyl-indazole

A mixture of 7-bromo-3-(2,6-dibenzyloxy-3-pyridyl)-1-methyl-indazole (200 mg, 399.69 μmol, 1.0 equiv.), 4-(3,3-dimethoxypropyl)piperidine (97.31 mg, 519.60 μmol, 1.3 equiv.), Cs₂CO₃ (390.68 mg, 1.20 mmol, 3.0 equiv.), and 1,3-bis[2,6-bis(1-propylbutyl)phenyl]-4,5-dichloro-2H-imidazol-1-ium-2-ide; 3-chloropyridine; dichloropalladium (38.88 mg, 39.97 μmol, 0.1 equiv.) in 2-methyl-2-butanol (2 mL) was degassed and purged with N₂ three times, and then the mixture was stirred at 100° C. for 16 hours under N₂ atmosphere. The mixture was filtered and concentrated under reduced pressure to give a residue. The residue was purified by flash silica gel chromatography (Eluent of 0˜15% ethyl acetate/petroleum ether) to give 3-(2,6-dibenzyloxy-3-pyridyl)-7-[4-(3,3-dimethoxypropyl)-1-piperidyl]-1-methyl-indazole (100 mg, 148.3 μmol, 37.1% yield, 90.0% purity) as a yellow oil.

MS (ESI) m/z: 607.4 [M+H]⁺

¹H NMR (400 MHz, DMSO-d₆) δ=7.84 (d, J=8.0 Hz, 1H), 7.50-7.44 (m, 2H), 7.42-7.33 (m, 5H), 7.32-7.26 (m, 4H), 7.01-6.96 (m, 1H), 6.95-6.89 (m, 1H), 6.58 (d, J=8.0 Hz, 1H), 5.42 (d, J=5.6 Hz, 4H), 4.35 (t, J=5.6 Hz, 1H), 4.31 (s, 3H), 3.23 (s, 8H), 2.71-2.63 (m, 2H), 1.85-1.76 (m, 2H), 1.61-1.55 (m, 2H), 1.44-1.36 (m, 3H), 1.34-1.29 (m, 2H)

### Step E. Procedure for Preparation of 3-[7-[4-(3,3-dimethoxypropyl)-1-piperidyl]-1-methyl-indazol-3-yl]piperidine-2,6-dione

To a solution of 3-(2,6-dibenzyloxy-3-pyridyl)-7-[4-(3,3-dimethoxypropyl)-1-piperidyl]-1-methyl-indazole (100 mg, 164.81 μmol, 1.0 equiv.) in THE (1 mL) and EtOH (1 mL) was added Pd(OH)₂ (23.15 mg, 164.81 μmol, 1.0 equiv.) and Pd/C (50 mg, 10% purity) under N₂. The suspension was degassed under vacuum and purged with H₂ several times. The mixture was stirred under H₂ (15 psi) at 20° C. for 16 hours. The reaction mixture was filtered and concentrated under reduced pressure to give 3-[7-[4-(3,3-dimethoxypropyl)-1-piperidyl]-1-methyl-indazol-3-yl]piperidine-2,6-dione (65 mg, crude) as a yellow oil.

MS (ESI) m/z: 429.1 [M+H]⁺

¹H NMR (400 MHz, DMSO-d₆) δ=7.39-7.32 (m, 1H), 7.03-6.95 (m, 2H), 4.37-4.30 (m, 2H), 4.23 (s, 3H), 3.23 (s, 6H), 3.21-3.18 (m, 2H), 2.74-2.58 (m, 4H), 2.38-2.25 (m, 2H), 1.84-1.76 (m, 2H), 1.61-1.54 (m, 2H), 1.42-1.37 (m, 2H), 1.35-1.26 (m, 3H)

### Step F. Procedure for Preparation of 3-[1-[3-(2,6-dioxo-3-piperidyl)-1-methyl-indazol-7-yl]-4-piperidyl]propanal

A mixture of 3-[7-[4-(3,3-dimethoxypropyl)-1-piperidyl]-1-methyl-indazol-3-yl]piperidine-2,6-dione (60 mg, 140.02 μmol, 1.0 equiv.) and HCOOH (6.73 mg, 140.02 μmol, 1 mL, 1.0 equiv.) was stirred at 90° C. for 1 hour. The reaction mixture was concentrated under reduced pressure to give 3-[1-[3-(2,6-dioxo-3-piperidyl)-1-methyl-indazol-7-yl]-4-piperidyl]propanal (60 mg, crude) as a pink oil.

MS (ESI) m/z: 383.2 [M+H]⁺

### Step G. Procedure for Preparation of 6-[8-(1,3-benzothiazol-2-ylcarbamoyl)-3,4-dihydro-1H-isoquinolin-2-yl]-3-[3-[2-[1-[3-[1-[3-(2,6-dioxo-3-piperidyl)-1-methyl-indazol-7-yl]-4-piperidyl]propyl]-4-piperidyl]ethoxy]-2-methyl-phenyl]pyridine-2-carboxylic acid

To a solution of 6-[8-(1,3-benzothiazol-2-ylcarbamoyl)-3,4-dihydro-1H-isoquinolin-2-yl]-3-[2-methyl-3-[2-(4-piperidyl)ethoxy]phenyl]pyridine-2-carboxylic acid (80 mg, 123.50 μmol, 1.0 equiv.) and NaBH(OAc)₃ (235.57 mg, 1.11 mmol, 9.0 equiv.) in DCM (2 mL) and t-BuOH (0.5 mL), and then 3-[1-[3-(2,6-dioxo-3-piperidyl)-1-methyl-indazol-7-yl]-4-piperidyl]propanal (56.68 mg, 148.20 μmol, 1.2 equiv.) was added dropwise at 0° C. After addition, the mixture was warmed to 20° C. and stirred at 20° C. for 16 hours. The mixture was concentrated under reduced pressure to remove DCM and t-BuOH. The residue was purified by prep-HPLC to give 6-[8-(1,3-benzothiazol-2-ylcarbamoyl)-3,4-dihydro-1H-isoquinolin-2-yl]-3-[3-[2-[1-[3-[1-[3-(2,6-dioxo-3-piperidyl)-1-methyl-indazol-7-yl]-4-piperidyl]propyl]-4-piperidyl]ethoxy]-2-methyl-phenyl]pyridine-2-carboxylic acid (46.3 mg, 41.8 μmol, 33.8% yield, 95.6% purity, FA) as a white solid.

MS (ESI) m/z: 1015.5 [M+H]⁺

¹H NMR (400 MHz, DMSO-d₆) δ=10.89 (s, 1H), 8.01 (d, J=8.0 Hz, 1H), 7.77 (d, J=8.0 Hz, 1H), 7.61 (d, J=7.6 Hz, 1H), 7.48-7.42 (m, 1H), 7.42-7.29 (m, 5H), 7.10-7.04 (m, 1H), 7.03-6.96 (m, 2H), 6.89-6.82 (m, 2H), 6.66 (d, J=7.6 Hz, 1H), 4.95 (s, 2H), 4.35-4.31 (m, 1H), 4.22 (s, 3H), 3.98-3.94 (m, 2H), 3.90 (t, J=5.6 Hz, 2H), 3.24-3.15 (m, 4H), 3.0-2.95 (m, 4H), 2.69-2.59 (m, 4H), 2.35-2.28 (m, 1H), 2.25-2.10 (m, 3H), 1.90 (s, 3H), 1.82-1.74 (m, 2H), 1.71-1.60 (m, 4H), 1.54 (s, 3H), 1.36 (s, 3H), 1.29-1.14 (m, 4H)

### Example 116. Preparation of Compound 289a

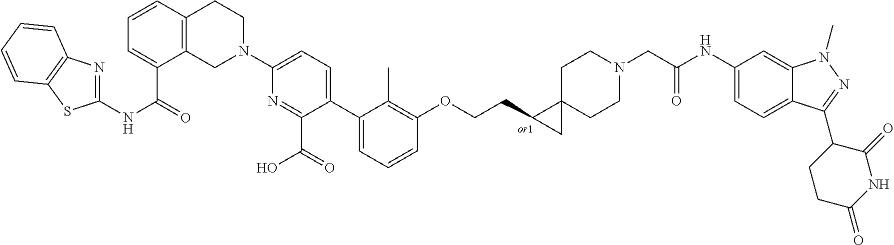 [SMILES: Cc1c(OCC[C@@H]2CC23CCN(CC(=O)Nc2ccc4c(C5CCC(=O)NC5=O)nn(C)c4c2)CC3)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(2-((1S)-6-(2-((3-(2,6-dioxopiperidin-3-yl)-1-methyl-1H-indazol-6-yl)amino)-2-oxoethyl)-6-azaspiro[2.5]octan-1-yl)ethoxy)-2-methylphenyl)picolinic acid

### [rel-6-(8-(benzo[d]thiazol-2-ylcarbamoyl)-3,4-dihydroisoquinolin-2(1H)-yl)-3-(3-(2-((1S)-6-(2-((3-(2,6-dioxopiperidin-3-yl)-1-methyl-1H-indazol-6-yl)amino)-2-oxoethyl)-6-azaspiro[2.5]octan-1-yl)ethoxy)-2-methylphenyl)picolinic acid]

### Step A. Procedure for Preparation of tert-butyl (S)-6-(8-(benzo[d]thiazol-2-ylcarbamoyl)-3,4-dihydroisoquinolin-2(1H)-yl)-3-(3-(2-(6-(2-ethoxy-2-oxoethyl)-6-azaspiro[2.5]octan-1-yl)ethoxy)-2-methylphenyl)picolinate

A mixture of ethyl (S)-2-(1-(2-(3-bromo-2-methylphenoxy)ethyl)-6-azaspiro[2.5]octan-6-yl)acetate (100 mg, 243.70 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (179.13 mg, 292.44 μmol, 1.2 equiv.), [2-(2-aminophenyl)phenyl]palladium(1+); bis(1-adamantyl)-butyl-phosphane; methanesulfonate (35.50 mg, 48.74 μmol, 0.2 equiv.), and K₂CO₃ (1.5 M, 487.40 μL, 3 equiv.) in dioxane (1 mL) and H₂O (1 mL) was degassed and purged with N₂ three times, and then the mixture was stirred at 100° C. for 1 hour under N₂ atmosphere under microwave. The combined organic layers were filtered and concentrated under reduced pressure to give a residue. The residue was purified by flash silica gel chromatography (Eluent of 0˜ 60% ethyl acetate/petroleum ether) to give tert-butyl (S)-6-(8-(benzo[d]thiazol-2-ylcarbamoyl)-3,4-dihydroisoquinolin-2(1H)-yl)-3-(3-(2-(6-(2-ethoxy-2-oxoethyl)-6-azaspiro[2.5]octan-1-yl)ethoxy)-2-methylphenyl)picolinate (140 mg, 171.6 μmol, 70.4% yield) as a yellow oil.

MS (ESI) m/z: 816.4 [M+H]⁺.

### Step B. Procedure for Preparation of (S)-2-(1-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethyl)-6-azaspiro[2.5]octan-6-yl)acetic acid

A mixture of tert-butyl (S)-6-(8-(benzo[d]thiazol-2-ylcarbamoyl)-3,4-dihydroisoquinolin-2(1H)-yl)-3-(3-(2-(6-(2-ethoxy-2-oxoethyl)-6-azaspiro[2.5]octan-1-yl)ethoxy)-2-methylphenyl)picolinate (140 mg, 171.57 μmol, 1 equiv.) and LiOH·H₂O (21.60 mg, 514.70 μmol, 3 equiv.) in THE (0.8 mL) and H₂O (0.4 mL) was stirred at 25° C. for 2 hours. The reaction mixture was diluted with H₂O (5 mL) and concentrated as a turbid liquid. Then the pH of the turbid liquid was adjusted to 3 with 1 N HCl (5 mL), and the mixture was extracted with DCM/MeOH (20:1), The combined organic layers were filtered and concentrated to give (S)-2-(1-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethyl)-6-azaspiro[2.5]octan-6-yl)acetic acid (120 mg, 152.3 μmol, 88.8% yield) as a yellow solid.

MS (ESI) m/z: 788.3 [M+H]⁺

### Step C. Procedure for Preparation of tert-butyl 6-(8-(benzo[d]thiazol-2-ylcarbamoyl)-3,4-dihydroisoquinolin-2(1H)-yl)-3-(3-(2-((1S)-6-(2-((3-(2,6-dioxopiperidin-3-yl)-1-methyl-11H-indazol-6-yl)amino)-2-oxoethyl)-6-azaspiro[2.5]octan-1-yl)ethoxy)-2-methylphenyl)picolinate

A mixture of (S)-2-(1-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethyl)-6-azaspiro[2.5]octan-6-yl)acetic acid (110 mg, 139.60 μmol, 1 equiv.), 3-(6-amino-1-methyl-indazol-3-yl)piperidine-2,6-dione (43.27 mg, 167.52 μmol, 1.2 equiv.), and EDCI (40.14 mg, 209.40 μmol, 1.5 equiv.) in pyridine (1.2 mL) was stirred at 25° C. for 2 hours. The mixture was diluted with H₂O (10 mL×3) and extracted with ethyl acetate (8 mL×3). The combined organic layers were filtered and concentrated under reduced pressure to give tert-butyl 6-(8-(benzo[d]thiazol-2-ylcarbamoyl)-3,4-dihydroisoquinolin-2(1H)-yl)-3-(3-(2-((1S)-6-(2-((3-(2,6-dioxopiperidin-3-yl)-1-methyl-1H-indazol-6-yl)amino)-2-oxoethyl)-6-azaspiro[2.5]octan-1-yl)ethoxy)-2-methylphenyl)picolinate (100 mg, crude) as a yellow solid.

MS (ESI) m/z: 1028.6 [M+H]⁺

### Step D. Procedure for Preparation of 6-(8-(benzo[d]thiazol-2-ylcarbamoyl)-3,4-dihydroisoquinolin-2(1H)-yl)-3-(3-(2-((1S)-6-(2-((3-(2,6-dioxopiperidin-3-yl)-1-methyl-11H-indazol-6-yl)amino)-2-oxoethyl)-6-azaspiro[2.5]octan-1-yl)ethoxy)-2-methylphenyl)picolinic acid [rel-6-(8-(benzo[d]thiazol-2-ylcarbamoyl)-3,4-dihydroisoquinolin-2(1H)-yl)-3-(3-(2-((1S)-6-(2-((3-(2,6-dioxopiperidin-3-yl)-1-methyl-1H-indazol-6-yl)amino)-2-oxoethyl)-6-azaspiro[2.5]octan-1-yl)ethoxy)-2-methylphenyl)picolinic acid]

A mixture of tert-butyl 6-(8-(benzo[d]thiazol-2-ylcarbamoyl)-3,4-dihydroisoquinolin-2(1H)-yl)-3-(3-(2-((1S)-6-(2-((3-(2,6-dioxopiperidin-3-yl)-1-methyl-1H-indazol-6-yl)amino)-2-oxoethyl)-6-azaspiro[2.5]octan-1-yl)ethoxy)-2-methylphenyl)picolinate (100 mg, 97.26 μmol, 1 equiv.) in TFA (0.1 mL) and DCM (0.1 mL) was stirred at 25° C. for 16 hours. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(2-((1S)-6-(2-((3-(2,6-dioxopiperidin-3-yl)-1-methyl-1H-indazol-6-yl)amino)-2-oxoethyl)-6-azaspiro[2.5]octan-1-yl)ethoxy)-2-methylphenyl)picolinic acid [rel-6-(8-(benzo[d]thiazol-2-ylcarbamoyl)-3,4-dihydroisoquinolin-2(1H)-yl)-3-(3-(2-((1S)-6-(2-((3-(2,6-dioxopiperidin-3-yl)-1-methyl-1H-indazol-6-yl)amino)-2-oxoethyl)-6-azaspiro[2.5]octan-1-yl)ethoxy)-2-methylphenyl)picolinic acid] (98.4% purity) as a yellow solid.

MS (ESI) m/z: 972.4 [M+H]⁺

¹H NMR (400 MHz, DMSO-d₆) δ=12.94-12.80 (m, 1H), 10.93-10.82 (m, 1H), 10.19-9.74 (m, 1H), 8.06-7.99 (m, 2H), 7.81-7.76 (m, 1H), 7.67-7.60 (m, 2H), 7.47-7.34 (m, 5H), 7.21-7.07 (m, 2H), 6.98-6.89 (m, 2H), 6.63 (d, J=7.6 Hz, 1H), 4.97 (s, 2H), 4.33 (dd, J=5.2, 9.6 Hz, 1H), 4.11-4.03 (m, 2H), 3.94-3.90 (m, 5H), 3.50-3.39 (m, 4H), 3.02 (t, J=5.2 Hz, 2H), 2.68-2.54 (m, 6H), 2.36-2.29 (m, 1H), 2.21-2.14 (m, 1H), 1.92 (s, 3H), 1.73-1.54 (m, 2H), 1.47-0.98 (m, 2H), 0.94-0.72 (m, 1H), 0.66-0.41 (m, 1H), 0.36-0.17 (m, 1H)

### Example 117. Preparation of Compound 101

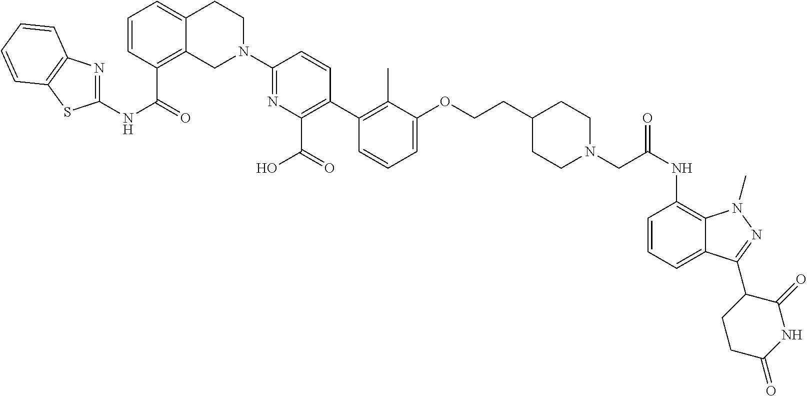 [SMILES: Cc1c(OCCC2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[2-[1-[2-[[3-(2,6-dioxo-3-piperidyl)-1-methyl-indazol-7-yl]amino]-2-oxo-ethyl]-4-piperidyl]ethoxy]-2-methyl-phenyl]pyridine-2-carboxylic acid

### Step A. Procedure for Preparation of 2-(tert-butyl) 8-methyl 3,4-dihydroisoquinoline-2,8(1H)-dicarboxylate [SMILEs: O═C(N1CC2=C(C(OC)═O)C═CC═C₂CC1)OC(C)(C)C]

To a solution of 2-tert-butoxycarbonyl-3,4-dihydro-1H-isoquinoline-8-carboxylic acid (62 g, 223.57 mmol, 1 equiv.) in DMF (600 mL) was added Mel (38.08 g, 268.29 mmol, 16.70 mL, 1.2 equiv.) and K₂CO₃ (61.80 g, 447.15 mmol, 2 equiv.). The mixture was stirred at 50° C. for 2 hours. The mixture was poured into ice water (800 mL) (pH>8), extracted with EtOAc (300 mL×3). The organic layers were combined and washed with brine (300 mL×3), dried over Na₂SO₄, and concentrated in vacuo to give product. The crude product 2-(tert-butyl) 8-methyl 3,4-dihydroisoquinoline-2,8(1H)-dicarboxylate (66 g, crude) as a brown oil was used in the next step without further purification.

Step B. Procedure for Preparation of tert-butyl 8-(1,3-benzothiazol-2-ylcarbamoyl)-3,4-dihydro-1H-isoquinoline-2-carboxylate

To a solution of 1, 3-benzothiazol-2-amine (35.73 g, 237.87 mmol, 1.05 equiv.) in 2-MeTHF (350 mL) was added LiHMDS (1 M, 453.08 mL, 2 equiv.) at 0° C., and the solution was stirred at 0° C. for 30 min. Then a solution of 2-(tert-butyl) 8-methyl 3,4-dihydroisoquinoline-2,8(1H)-dicarboxylate (66.87 g, 226.54 mmol, 98.7% purity, 1 equiv.) in 2-MeTHF (350 mL) was added to the above solution at 0° C. and stirred for 30 min again. The resulting mixture was warmed to 60° C. and stirred for 17 hours. The reaction mixture was poured into sat.NH₄Cl (1 L), extracted with EtOAc (500 mL×3). The combined organic layer was washed with brine, dried over Na₂SO₄, concentrated in vacuo to give a crude product. The crude product was purified by re-crystallization from EtOAc (500 mL) at 25° C. The filtrate was concentrated in vacuo to give a residue. The residue was dissolved EtOAc (300 mL), the solution was washed with 10% citric acid (100 mL×3) and brine (150 mL), dried over Na₂SO₄, and concentrated under reduced pressure to give a residue. The residue was purified by re-crystallization from ethyl acetate/petroleum ether=1/1 (600 m1) at 25° C. The second recrystallized mother liquor was concentrated in vacuo to give residue. The residue was purified by flash silica gel chromatography (Eluent of 0˜40% DCM/petroleum ether) to give title product (5 g). Combined three batch products to afford tert-butyl 8-(1,3-benzothiazol-2-ylcarbamoyl)-3,4-dihydro-1H-isoquinoline-2-carboxylate (66.7 g, 162.88 mmol, 72.85% yield of two steps) as a light yellow solid.

MS (ESI) m/z: 410.0 [M+H]⁺.

¹H NMR (400 MHz, CDCl₃) δ=7.87-7.79 (m, 1H), 7.51 (d, J=7.4 Hz, 1H), 7.33-7.26 (m, 3H), 7.22 (d, J=7.4 Hz, 1H), 7.17-7.10 (m, 1H), 4.91 (s, 2H), 3.64 (s, 2H), 2.88 (s, 2H), 1.57-1.34 (m, 9H)

### Step C. Procedure for Preparation of N-(1,3-benzothiazol-2-yl)-1,2,3,4-tetrahydroisoquinoline-8-carboxamide

To a mixture of tert-butyl 8-(1,3-benzothiazol-2-ylcarbamoyl)-3,4-dihydro-1H-isoquinoline-2-carboxylate (61.7 g, 150.67 mmol, 1 equiv.) in DCM (1000 mL) was added HCl/dioxane (600 mL). The mixture was stirred at 25° C. for 6 hours. TLC (petroleum ether:ethyl acetate=3:1, R_{f}=0) showed the reaction was completed. The reaction mixture was concentrated in vacuo to give N-(1,3-benzothiazol-2-yl)-1,2,3,4-tetrahydroisoquinoline-8-carboxamide (59 g, crude, 2HCl) as a white solid. The solid was used in the next step without further purification.

¹H NMR (400 MHz, DMSO-d₆) δ=9.63 (br s, 2H), 8.02 (d, J=7.8 Hz, 1H), 7.87-7.61 (m, 2H), 7.55-7.22 (m, 4H), 4.47-4.36 (m, 2H), 3.56 (s, 3H), 3.43-3.28 (m, 2H), 3.11 (t, J=5.8 Hz, 2H)

### Step D. Procedure for Preparation of tert-butyl 6-[8-(1,3-benzothiazol-2-ylcarbamoyl)-3,4-dihydro-1H-isoquinolin-2-yl]-3-bromo-pyridine-2-carboxylate

To a mixture of N-(1,3-benzothiazol-2-yl)-1,2,3,4-tetrahydroisoquinoline-8-carboxamide (30 g, 73.76 mmol, 94% purity, 1 equiv., 2HCl) in DMA (300 mL) was added Cs₂CO₃ (120.17 g, 368.81 mmol, 5 equiv.) and tert-butyl 3-bromo-6-chloro-pyridine-2-carboxylate (21.80 g, 74.50 mmol, 1.01 equiv.). The mixture was stirred at 120° C. for 12 hours. Then tert-butyl 3-bromo-6-chloro-pyridine-2-carboxylate (5.8 g, 19.83 mmol, 2.69 e¹ equiv.) was added to the reaction mixture, and the resulting mixture was stirred at 120° C. for 7 hours. The reaction mixture was cooled to room temperature, and poured onto ice water (1 L). The mixture was stirred for 10 min and filtered. The cake was washed with EtOH and collected. The cake was triturated with EtOH (200 mL) at 25° C. for 30 min to give tert-butyl 6-[8-(1,3-benzothiazol-2-ylcarbamoyl)-3,4-dihydro-1H-isoquinolin-2-yl]-3-bromo-pyridine-2-carboxylate (24.6 g, 43.50 mmol, 58.98% yield) was obtained as a white solid.

MS (ESI) m/z: 565.0 [M+H]⁺

¹H NMR (400 MHz, DMSO-d₆) δ=12.70 (brs, 1H), 8.01 (d, J=7.8 Hz, 1H), 7.77 (dd, J=2.8, 8.4 Hz, 2H), 7.58 (d, J=7.4 Hz, 1H), 7.50-7.39 (m, 2H), 7.39-7.30 (m, 2H), 6.86 (d, J=9.2 Hz, 1H), 4.93 (s, 2H), 3.77 (t, J=6.0 Hz, 2H), 3.00 (t, J=5.8 Hz, 2H), 1.34 (s, 9H)

### Step E. Procedure for Preparation of tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl) pyridine-2-carboxylate

tert-Butyl 6-[8-(1,3-benzothiazol-2-ylcarbamoyl)-3,4-dihydro-1H-isoquinolin-2-yl]-3-bromo-pyridine-2-carboxylate (1.5 g, 2.65 mmol, 1 equiv.), 4,4,5,5-tetramethyl-1,3,2-dioxaborolane (746.84 mg, 5.84 mmol, 846.76 μL, 2.2 equiv.), TEA (836.05 mg, 8.26 mmol, 1.15 mL, 3.11 equiv.), and Pd(dppf)Cl₂·CH₂Cl₂ (108.31 mg, 132.63 μmol, 0.05 equiv.) were taken up into a microwave tube in CH₃CN (15 mL) and THE (5 mL). The sealed tube was heated at 100° C. for 30 min under microwave. The reaction mixture was poured into water (200 mL) and extracted with ethyl acetate (150 mL×2). The combined organic phase was concentrated in vacuo to give a residue. The residue was purified by column chromatography (SiO₂, petroleum ether/ethyl acetate=6/1 to 1/2) to afford title product. The compound tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl) pyridine-2-carboxylate (9.2 g, 14.27 mmol, 55.0% yield, 95% purity) was obtained as a white solid.

MS (ESI) m/z: 613.7 [M+H]⁺.

¹H NMR (400 MHz, DMSO-d₆) δ=12.84 (s, 1H), 8.03 (d, J=7.6 Hz, 1H), 7.78 (d, J=8.2 Hz, 1H), 7.68 (d, J=8.6 Hz, 1H), 7.56 (d, J=7.2 Hz, 1H), 7.50-7.39 (m, 2H), 7.38-7.30 (m, 2H), 6.86 (d, J=8.8 Hz, 1H), 5.01 (s, 2H), 3.82 (t, J=5.8 Hz, 2H), 3.00 (t, J=5.8 Hz, 2H), 1.28 (s, 9H), 1.22 (s, 12H)

### Step F. Procedure for Preparation of tert-butyl 4-[2-(3-bromo-2-methyl-phenoxy)ethyl]piperidine-1-carboxylate

A mixture of tert-butyl 4-(2-hydroxyethyl)piperidine-1-carboxylate (5 g, 21.80 mmol, 1 equiv.), 3-bromo-2-methyl-phenol (4.08 g, 21.80 mmol, 1 equiv.) and PPh₃ (7.43 g, 28.35 mmol, 1.3 equiv.) in THE (40 mL) was degassed and purged with N₂ for three times, and then the DIAD (5.29 g, 26.16 mmol, 5.09 mL, 1.2 equiv.) was added dropwise at 0° C. The mixture was stirred at 25° C. for 12 hours under N₂ atmosphere. The mixture solution diluted with H₂O (100 mL) and extracted with EtOAC 180 mL (60 mL×3). The combined organic layers were washed with H₂O 180 mL (60 mL×3), dried over Na₂SO₄, filtered, and concentrated under reduced pressure to give a residue. The residue was purified by column chromatography (petroleum ether/ethyl acetate=100/1 to 5/1). The compound tert-butyl 4-[2-(3-bromo-2-methyl-phenoxy)ethyl] piperidine-1-carboxylate (8.2 g, 18.5 mmol, 84.9% yield, 90% purity) was obtained as a brown oil.

MS (ESI) m/z: 344.0 [M-56]⁺

### Step G. Procedure for Preparation of 4-[2-(3-bromo-2-methyl-phenoxy)ethyl]piperidine

To a solution of tert-butyl 4-[2-(3-bromo-2-methyl-phenoxy)ethyl]piperidine-1-carboxylate (8.2 g, 20.59 mmol, 1 equiv.) was added HCl/dioxane (100 mL). The mixture was stirred at 25° C. for 2 hours. The reaction mixture was concentrated under reduced pressure to remove solvent. The compound 4-[2-(3-bromo-2-methyl-phenoxy)ethyl]piperidine (6 g, crude) was obtained as a white solid.

Step H. Procedure for Preparation of ethyl 2-[4-[2-(3-bromo-2-methyl-phenoxy)ethyl]-1-piperidyl]acetate

To a solution of 4-[2-(3-bromo-2-methyl-phenoxy)ethyl]piperidine (3 g, 10.06 mmol, 1 equiv.) in CH₃CN (30 mL) was added ethyl 2-bromoacetate (2.02 g, 12.07 mmol, 1.34 mL, 1.2 equiv.) and TEA (3.05 g, 30.18 mmol, 4.20 mL, 3 equiv.). The mixture was stirred at 20° C. for 12 hours. The mixture solution was diluted with H₂O (30 mL) and extracted with ethyl acetate (60 mL×3). The combined organic layers were washed with H₂O (10 mL×3), dried over Na₂SO₄, filtered, and concentrated under reduced pressure to give a residue. The residue was purified by column chromatography (petroleum ether/ethyl acetate=100/1 to 5/1). The compound ethyl 2-[4-[2-(3-bromo-2-methyl-phenoxy)ethyl]-1-piperidyl]acetate (3 g, 7.7 mmol, 76.8% yield, 99% purity) was obtained as a white liquid.

MS (ESI) m/z: 385.8 [M+H]⁺(⁸⁰Br)

### Step L Procedure for preparation of tert-butyl 6-[8-(1,3-benzothiazol-2-ylcarbamoyl)-3,4-dihydro-1H-isoquinolin-2-yl]-3-[3-[2-[1-(2-ethoxy-2-oxo-ethyl)-4-piperidyl]ethoxy]-2-methyl-phenyl]pyridine-2-carboxylate

To a solution of ethyl 2-[4-[2-(3-bromo-2-methyl-phenoxy)ethyl]-1-piperidyl]acetate (700 mg, 1.82 mmol, 1 equiv.) in 1,4-dioxane (8 mL) was added ditert-butyl(cyclopentyl)phosphane; dichloropalladium; iron (237.43 mg, 364.29 μmol, 0.2 equiv.), KF (1.5 M, 1.82 mL, 1.5 equiv.) and tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl) pyridine-2-carboxylate (1.23 g, 2.00 mmol, 1.1 equiv.). After addition, the mixture was stirred at 90° C. for 2 hours. The mixture solution diluted with H₂O (30 mL) and extracted with ethyl acetate (60 mL×3). The combined organic layers were washed with H₂O (10 mL×3), dried over Na₂SO₄, filtered, and concentrated under reduced pressure to give a residue. The residue was purified by column chromatography (petroleum ether/ethyl acetate=10/1 to 1/1) to give tert-butyl 6-[8-(1,3-benzothiazol-2-ylcarbamoyl)-3,4-dihydro-1H-isoquinolin-2-yl]-3-[3-[2-[1-(2-ethoxy-2-oxo-ethyl)-4-piperidyl]ethoxy]-2-methyl-phenyl]pyridine-2-carboxylate (600 mg, 721.5 μmol, 39.6% yield, 95% purity) as a brown solid.

MS (ESI) m/z: 790.5 [M+H]⁺

### Step J. Procedure for Preparation of 2-[4-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]ethyl]-1-piperidyl]acetic acid

To a solution of tert-butyl 6-[8-(1,3-benzothiazol-2-ylcarbamoyl)-3,4-dihydro-1H-isoquinolin-2-yl]-3-[3-[2-[1-(2-ethoxy-2-oxo-ethyl)-4-piperidyl]ethoxy]-2-methyl-phenyl]pyridine-2-carboxylate (600 mg, 759.51 μmol, 1 equiv.) in THE (3 mL) was added LiOH·H₂O (95.62 mg, 2.28 mmol, 3 equiv.) and H₂O (1 mL). The mixture was stirred at 20° C. for 1 hour. The pH was adjusted to 5 with citric acid. The mixture solution diluted with H₂O (10 mL) and extracted with DCM (10 mL×3). The combined organic layers were washed with H₂O (10 mL×3), dried over Na₂SO₄, filtered, and concentrated under reduced pressure to give a residue. The compound 2-[4-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]ethyl]-1-piperidyl]acetic acid (410 mg, crude) was obtained as a yellow solid.

MS (ESI) m/z: 762.3 [M+H]⁺

### Step K. Procedure for Preparation of tert-butyl 6-[8-(1,3-benzothiazol-2-ylcarbamoyl)-3,4-dihydro-1H-isoquinolin-2-yl]-3-[3-[2-[1-[2-[[3-(2,6-dioxo-3-piperidyl)-1-methyl-indazol-7-yl]amino]-2-oxo-ethyl]-4-piperidyl]ethoxy]-2-methyl-phenyl]pyridine-2-carboxylate

To a solution of 2-[4-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]ethyl]-1-piperidyl]acetic acid (110 mg, 144.37 μmol, 1 equiv.) and 3-(7-amino-1-methyl-indazol-3-yl)piperidine-2,6-dione (48.47 mg, 187.68 μmol, 1.3 equiv.) in DMF (1 mL) was added HATU (71.36 mg, 187.68 μmol, 1.3 equiv.) and DIEA (55.98 mg, 433.11 μmol, 75.44 μL, 3 equiv.). The mixture was stirred at 40° C. for 2 hours. The reaction mixture was treated with H₂O (10 mL), dried over Na₂SO₄, filtered, and concentrated under reduced pressure to give a crude tert-butyl 6-[8-(1,3-benzothiazol-2-ylcarbamoyl)-3,4-dihydro-1H-isoquinolin-2-yl]-3-[3-[2-[1-[2-[[3-(2,6-dioxo-3-piperidyl)-1-methyl-indazol-7-yl]amino]-2-oxo-ethyl]-4-piperidyl]ethoxy]-2-methyl-phenyl]pyridine-2-carboxylate (120 mg, crude) as a white solid.

MS (ESI) m/z: 1002.7 [M+H]+

### Step L. Procedure for Preparation of 6-[8-(1,3-benzothiazol-2-ylcarbamoyl)-3,4-dihydro-1H-isoquinolin-2-yl]-3-[3-[2-[1-[2-[[3-(2,6-dioxo-3-piperidyl)-1-methyl-indazol-7-yl]amino]-2-oxo-ethyl]-4-piperidyl]ethoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[2-[1-[2-[[3-(2,6-dioxo-3-piperidyl)-1-methyl-indazol-7-yl]amino]-2-oxo-ethyl]-4-piperidyl]ethoxy]-2-methyl-phenyl]pyridine-2-carboxylate (70 mg, 69.85 μmol, 1 equiv.) in DCM (1 mL) was added TFA (1.54 g, 13.51 mmol, 1 mL, 193.37 equiv.). The mixture was stirred at 20° C. for 2 hours. The reaction mixture was filtered and concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[2-[1-[2-[[3-(2,6-dioxo-3-piperidyl)-1-methyl-indazol-7-yl]amino]-2-oxo-ethyl]-4-piperidyl]ethoxy]-2-methyl-phenyl]pyridine-2-carboxylic acid (23.1 mg, 24.4 μmol, 35% yield, 99% purity) as a white solid.

MS (ESI) m/z: 946.5 [M+H]⁺

¹H NMR (400 MHz, DMSO-d₆) δ=10.9 (s, 1H), 9.88 (s, 1H), 8.08-8.00 (m, 1H), 7.84-7.76 (m, 1H), 7.64-7.56 (m, 2H), 7.52-7.28 (m, 7H), 7.12-7.04 (m, 2H), 6.96-6.88 (m, 2H), 6.60 (d, J=1.6 Hz, 1H), 4.98 (s, 2H), 4.40-4.32 (m, 1H), 4.12-4.08 (m, 3H), 4.04-3.96 (m, 2H), 3.92-3.88 (m, 2H), 3.16-3.08 (m, 2H), 3.04-3.00 (m, 2H), 2.96-2.92 (m, 2H), 2.72-2.60 (m, 1H), 2.36-2.32 (m, 1H), 2.24-2.12 (m, 4H), 1.92 (s, 3H), 1.76-1.68 (m, 4H), 1.56-1.48 (m, 1H), 1.44-1.36 (m, 2H)

### Example 118. Preparation of Compound 102

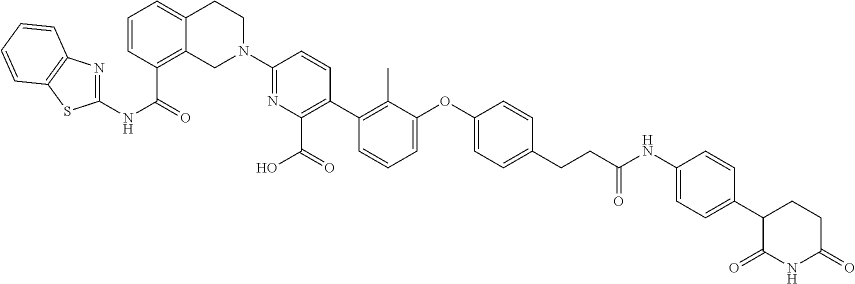 [SMILES: Cc1c(Oc2ccc(CCC(=O)Nc3ccc(C4CCC(=O)NC4=O)cc3)cc2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[4-[3-[4-(2,6-dioxo-3-piperidyl)anilino]-3-oxo-propyl]phenoxy]-2-methyl-phenyl]pyridine-2-carboxylic acid

### Step A. Procedure for Preparation of 2,6-dibenzyloxy-3-(4-nitrophenyl)pyridine

To a solution of (2, 6-dibenzyloxy-3-pyridyl)boronic acid (4 g, 11.93 mmol, 1 equiv.), 1-bromo-4-nitro-benzene (2.65 g, 13.13 mmol, 1.1 equiv.) in 1,4-dioxane (40 mL) and H₂O (4 mL) was added Pd(dppf)Cl₂ (873.26 mg, 1.19 mmol, 0.1 equiv.) and Na₂CO₃ (3.79 g, 35.80 mmol, 3 equiv.). The mixture was stirred at 100° C. for 2 hours. The reaction mixture was filtered and concentrated under reduced pressure to give a residue. The residue was purified by column chromatography (SiO₂, petroleum ether/ethyl acetate=100/1 to 50/1) to give 2,6-dibenzyloxy-3-(4-nitrophenyl)pyridine (4.6 g, 11.1 mmol, 93.4% yield) as a yellow solid.

¹H NMR (400 MHz, CDCl₃) δ=8.15 (d, J=8.8 Hz, 2H), 7.65 (d, J=8.8 Hz, 2H), 7.58 (d, J=8.0 Hz, 1H), 7.39-7.21 (m, 10H), 6.46 (d, J=8.0 Hz, 1H), 5.37 (s, 2H), 5.33 (s, 2H)

### Step B. Procedure for Preparation of 3-(4-aminophenyl)piperidine-2,6-dione

To a solution of 2,6-dibenzyloxy-3-(4-nitrophenyl)pyridine (4.6 g, 11.15 mmol, 1 equiv.) in THF (40 mL) and EtOH (30 mL) was added Pd(OH)₂ (1.57 g, 1.12 mmol, 10% purity, 0.1 equiv.) and Pd/C (1.17 g, 1.12 mmol, 10% purity, 0.1 equiv.) under N₂ atmosphere. The suspension was degassed and purged with H₂ three times. The mixture was stirred under H₂ (50 Psi or atm.) at 50° C. for 12 hours. The reaction mixture was filtered and concentrated under reduced pressure to give a residue. The residue was purified by column chromatography (SiO₂, dichloromethane:methanol=100/1 to 20/1) to give 3-(4-aminophenyl)piperidine-2,6-dione (900 mg, 4.4 mmol, 39.5% yield) as a white solid.

¹H NMR (400 MHz, DMSO-d₆) δ=10.79-10.68 (m, 1H), 6.92-6.78 (m, 2H), 6.59-6.45 (m, 2H), 4.98 (b s, 2H), 3.66-3.52 (m, 1H), 2.65-2.55 (m, 2H), 2.12-1.94 (m, 2H)

### Step C. Procedure for Preparation of methyl 3-[4-(3-bromo-2-methyl-phenoxy) phenyl]propanoate

To a solution of methyl 3-(4-hydroxyphenyl) propanoate (1 g, 5.55 mmol, 1 equiv.) in NMP (10 mL) was added 1, 3-dibromo-2-methyl-benzene (4.16 g, 16.65 mmol, 3 equiv.), CuI (528.44 mg, 2.77 mmol, 0.5 equiv.), Cs₂CO₃ (2.17 g, 6.66 mmol, 1.2 equiv.), and dipivaloylmethane (255.65 mg, 1.39 mmol, 285.65 μL, 0.25 equiv.) at 25° C. The reaction mixture was stirred at 120° C. for 16 hours under N₂. The reaction mixture was diluted with water (20 mL) and extracted with EtOAc (30 mL×3). The combined organic layers were washed with brine (20 mL×3), dried over Na₂SO₄, filtered, and concentrated under reduced pressure to give a residue. The residue was purified by flash silica gel chromatography (Eluent of 0-10% ethyl acetate/petroleum ether). The compound methyl 3-[4-(3-bromo-2-methyl-phenoxy) phenyl]propanoate (1.68 g, 4.33 mmol, 78.02% yield, 90% purity) was obtained as a yellow oil.

MS (ESI) m/z: 350.7 [M+H]⁺

¹H NMR (400 MHz, CD₃OD) δ=7.35 (d, J=7.6 Hz, 1H), 7.21-7.16 (m, 2H), 7.06 (t, J=8.0 Hz, 1H), 6.85-6.77 (m, 3H), 3.67-3.61 (m, 3H), 2.89 (t, J=7.6 Hz, 2H), 2.65-2.59 (m, 2H), 2.30 (s, 3H)

### Step D. Procedure for Preparation of tert-butyl 6-[8-(1, 3-benzothiazol-2-ylcarbamoyl)-3, 4-dihydro-1H-isoquinolin-2-yl]-3-[3-[4-(3-methoxy-3-oxo-propyl) phenoxy]-2-methyl-phenyl]pyridine-2-carboxylate

To a solution of methyl 3-[4-(3-bromo-2-methyl-phenoxy) phenyl]propanoate (1 g, 2.86 mmol, 1 equiv.) in dioxane (10 mL) was added tert-butyl 6-[8-(1, 3-benzothiazol-2-ylcarbamoyl)-3, 4-dihydro-1H-isoquinolin-2-yl]-3-(4, 4, 5, 5-tetramethyl-1, 3, 2-dioxaborolan-2-yl) pyridine-2-carboxylate (1.93 g, 3.15 mmol, 1.1 equiv.), Ad₂nBuP Pd G₃ (cataCXium® A Pd G₃) (417.09 mg, 572.71 μmol, 0.2 equiv.), and K₃PO₄ (1.5 M, 5.73 mL, 3 equiv.) at 25° C. The reaction mixture was purged with N₂ 3 time and stirred under N₂ at 80° C. for 16 hours. The reaction mixture was diluted with water (10 mL) and extracted with EtOAc (20 mL×3). The combined organic layers were washed with brine (20 mL×1), dried over Na₂SO₄, filtered, and concentrated under reduced pressure to give a residue. The residue was purified by flash silica gel chromatography (Eluent of 0-50% ethyl acetate/petroleum ether). The compound tert-butyl 6-[8-(1, 3-benzothiazol-2-ylcarbamoyl)-3, 4-dihydro-1H-isoquinolin-2-yl]-3-[3-[4-(3-methoxy-3-oxo-propyl) phenoxy]-2-methyl-phenyl]pyridine-2-carboxylate (1 g, 1.30 mmol, 45.34% yield, 98% purity) was obtained as a yellow solid.

MS (ESI) m/z: 755.3 [M+H]⁺.

### Step E. Procedure for Preparation of 3-[4-[3-[6-[8-(1, 3-benzothiazol-2-ylcarbamoyl)-3, 4-dihydro-1H-isoquinolin-2-yl]-2-tert-butoxycarbonyl-3-pyridyl]-2-methyl-phenoxy]phenyl]propanoic acid

To a solution of tert-butyl 6-[8-(1, 3-benzothiazol-2-ylcarbamoyl)-3, 4-dihydro-1H-isoquinolin-2-yl]-3-[3-[4-(3-methoxy-3-oxo-propyl) phenoxy]-2-methyl-phenyl]pyridine-2-carboxylate (500 mg, 662.35 μmol, 1 eq) in THE (5 mL) was added LiOH·H₂O (1 M, 1.99 mL, 3 eq) at 25° C. The reaction mixture was stirred at 25° C. for 2 hours. The reaction mixture was acidified with 1N HCl to pH=6, filtered, and concentrated under reduced pressure to give a residue. The compound 3-[4-[3-[6-[8-(1, 3-benzothiazol-2-ylcarbamoyl)-3, 4-dihydro-1H-isoquinolin-2-yl]-2-tert-butoxycarbonyl-3-pyridyl]-2-methyl-phenoxy]phenyl]propanoic acid (616 mg, crude) was obtained as a white solid.

MS (ESI) m/z: 741.3 [M+H]⁺.

### Step F. Procedure for Preparation of tert-butyl 6-[8-(1,3-benzothiazol-2-ylcarbamoyl)-3,4-dihydro-1H-isoquinolin-2-yl]-3-[3-[4-[3-[4-(2,6-dioxo-3-piperidyl)anilino]-3-oxo-propyl]phenoxy]-2-methyl-phenyl]pyridine-2-carboxylate

To a solution of 3-[4-[3-[6-[8-(1, 3-benzothiazol-2-ylcarbamoyl)-3, 4-dihydro-1H-isoquinolin-2-yl]-2-tert-butoxycarbonyl-3-pyridyl]-2-methyl-phenoxy]phenyl]propanoic acid (150 mg, 202.47 μmol, 1 equiv.) in DMF (1 mL) was added DIEA (78.50 mg, 607.40 μmol, 105.80 μL, 3 equiv.), HATU (92.38 mg, 242.96 μmol, 1.2 equiv.), and 3-(4-aminophenyl)piperidine-2,6-dione (45.48 mg, 222.71 μmol, 1.1 equiv.). The mixture was stirred at 25° C. for 1 hour. The reaction mixture was added slowly to water (10 mL). The resulting mixture was filtered, and the filtrate was concentrated under reduced pressure to give tert-butyl 6-[8-(1,3-benzothiazol-2-ylcarbamoyl)-3,4-dihydro-1H-isoquinolin-2-yl]-3-[3-[4-[3-[4-(2,6-dioxo-3-piperidyl)anilino]-3-oxo-propyl]phenoxy]-2-methyl-phenyl]pyridine-2-carboxylate (100 mg, 107.9 μmol, 53.3% yield) as a white solid was used into the next step without further purification.

MS (ESI) m/z: 927.4 [M+H]⁺.

### Step G. Procedure for Preparation of 6-[8-(1,3-benzothiazol-2-ylcarbamoyl)-3,4-dihydro-1H-isoquinolin-2-yl]-3-[3-[4-[3-[4-(2,6-dioxo-3-piperidyl)anilino]-3-oxo-propyl]phenoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[4-[3-[4-(2,6-dioxo-3-piperidyl)anilino]-3-oxo-propyl]phenoxy]-2-methyl-phenyl]pyridine-2-carboxylate (90 mg, 97.08 μmol, 1 equiv.) in DCM (1 mL) was added TFA (1.54 g, 13.51 mmol, 1 mL, 139.13 equiv.). The mixture was stirred at 40° C. for 1 hour. The reaction mixture was concentrated under reduced pressure to remove solvent. The residue was purified by prep-HPLC to give 6-[8-(1,3-benzothiazol-2-ylcarbamoyl)-3,4-dihydro-1H-isoquinolin-2-yl]-3-[3-[4-[3-[4-(2,6-dioxo-3-piperidyl)anilino]-3-oxo-propyl]phenoxy]-2-methyl-phenyl]pyridine-2-carboxylic acid (29.2 mg, 31.7 μmol, 32.6% yield, 94.4% purity) as a yellow solid.

MS (ESI) m/z: 871.3 [M+H]⁺.

¹H NMR (400 MHz, DMSO) δ=12.92-12.77 (m, 1H), 10.84-10.77 (m, 1H), 9.89 (s, 1H), 8.04 (d, J=8.0 Hz, 1H), 7.83-7.76 (m, 1H), 7.63 (d, J=7.6 Hz, 1H), 7.55-7.50 (m, 3H), 7.48-7.44 (m, 2H), 7.40-7.33 (m, 2H), 7.25-7.09 (m, 6H), 7.03-6.99 (m, 1H), 6.88 (d, J=7.6 Hz, 1H), 6.85-6.79 (m, 3H), 5.00 (s, 2H), 3.94 (t, J=5.6 Hz, 2H), 3.81-3.77 (m, 1H), 3.04 (t, J=5.6 Hz, 2H), 2.87 (d, J=7.6 Hz, 2H), 2.60 (s, 2H), 2.21-2.09 (m, 2H), 2.06-1.96 (m, 2H), 1.90 (s, 3H)

### Example 119. Preparation of Compound 103

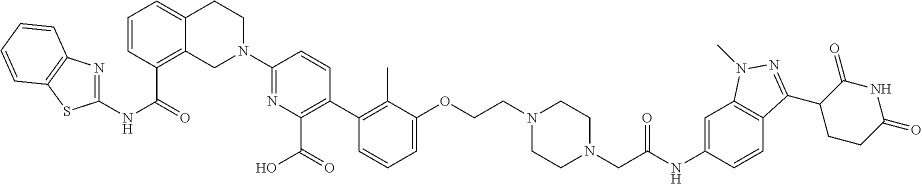 [SMILES: Cc1c(OCCN2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[2-[4-[2-[[3-(2,6-dioxo-3-piperidyl)-1-methyl-indazol-6-yl]amino]-2-oxo-ethyl]piperazin-1-yl]ethoxy]-2-methyl-phenyl]pyridine-2-carboxylic acid

### Step A. Procedure for Preparation of 1-bromo-3-(2-bromoethoxy)-2-methyl-benzene

To a solution of 1,2-dibromoethane (36.16 g, 192.48 mmol, 14.52 mL, 8 equiv.) in CH₃CN (40 mL) was added Cs₂CO₃ (15.68 g, 48.12 mmol, 2 equiv.) and 3-bromo-2-methyl-phenol (4.5 g, 24.06 mmol, 1 equiv.). The mixture was stirred at 80° C. for 15 hours. The solution was filtered and concentrated under reduced pressure to give a residue. The residue was purified by column chromatography (SiO₂, petroleum ether/ethyl acetate=50/1 to 20/1) to obtain 1-bromo-3-(2-bromoethoxy)-2-methyl-benzene (4.5 g, 15.3 mmol, 63.6% yield) as a yellow oil.

¹H NMR (400 MHz, CDCl₃) δ=7.20 (d, J=8.0 Hz, 1H), 7.01 (t, J=8.0 Hz, 1H), 6.76 (d, J=8.0 Hz, 1H), 4.29 (t, J=6.0 Hz, 2H), 3.67 (t, J=6.0 Hz, 2H), 2.36 (s, 3H).

### Step B. Procedure for Preparation of ethyl 2-[4-[2-(3-bromo-2-methyl-phenoxy)ethyl]piperazin-1-yl]acetate

To a solution of ethyl 2-piperazin-1-ylacetate (585.83 mg, 3.40 mmol, 1.00 equiv.) in DMF (10 mL) was added DIPEA (1.54 g, 11.91 mmol, 2.07 mL, 3.5 equiv.) and 1-bromo-3-(2-bromoethoxy)-2-methyl-benzene (1 g, 3.40 mmol, 1 equiv.). The mixture was stirred at 60° C. for 15 hours. The reaction mixture was partitioned between water (10 mL) and ethyl acetate (30 mL). The organic phase was separated and washed with brine (10 mL×2), dried over Na₂SO₄, filtered, and concentrated under reduced pressure to give a residue. The residue was purified by column chromatography (SiO₂, petroleum ether/ethyl acetate=100/1 to 1/100). The compound ethyl 2-[4-[2-(3-bromo-2-methyl-phenoxy)ethyl]piperazin-1-yl]acetate (2.6 g, 6.49 mmol, 95.42% yield, 96.2% purity) was obtained as a yellow oil.

MS (ESI) m/z: 385.1 [M+H]⁺

¹H NMR (400 MHz, CDCl₃) δ=7.15 (d, J=8.0 Hz, 1H), 6.99 (t, J=8.0 Hz, 1H), 6.76 (d, J=8.4 Hz, 1H), 4.19 (q, J=7.2 Hz, 2H), 4.12-4.08 (m, 2H), 3.21 (s, 2H), 2.86 (t, J=5.6 Hz, 2H), 2.72-2.61 (m, 8H), 2.30 (s, 3H), 1.29-1.26 (m, 3H).

### Step C. Procedure for Preparation of tert-butyl 6-[8-(1,3-benzothiazol-2-ylcarbamoyl)-3,4-dihydro-1H-isoquinolin-2-yl]-3-[3-[2-[4-(2-ethoxy-2-oxo-ethyl)piperazin-1-yl]ethoxy]-2-methyl-phenyl]pyridine-2-carboxylate

A mixture of ethyl 2-[4-[2-(3-bromo-2-methyl-phenoxy)ethyl]piperazin-1-yl]acetate (1 g, 2.60 mmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (1.27 g, 2.08 mmol, 0.8 equiv.), K₂CO₃ (1.5 M, 2.60 mL, 1.5 equiv.), and Ad₂nBuP Pd G₃(cataCXium® A Pd G₃) (378.03 mg, 519.08 μmol, 0.2 equiv.) in 1,4-dioxane (10 mL) was degassed and purged with N₂ three times. Then the mixture was stirred at 80° C. for 6 hours under N₂ atmosphere. The reaction mixture was concentrated under reduced pressure to give a residue. The residue was purified by column chromatography (SiO₂, DCM/MeOH=8:1). The compound tert-butyl 6-[8-(1,3-benzothiazol-2-ylcarbamoyl)-3,4-dihydro-1H-isoquinolin-2-yl]-3-[3-[2-[4-(2-ethoxy-2-oxo-ethyl)piperazin-1-yl]ethoxy]-2-methyl-phenyl]pyridine-2-carboxylate (1.3 g, 1.6 mmol, 63.3% yield) was obtained as a yellow oil.

MS (ESI) m/z: 791.3 [M+H]⁺

### Step D. Procedure for Preparation of 2-[4-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]ethyl]piperazin-1-yl]acetic acid

To a solution of tert-butyl 6-[8-(1,3-benzothiazol-2-ylcarbamoyl)-3,4-dihydro-1H-isoquinolin-2-yl]-3-[3-[2-[4-(2-ethoxy-2-oxo-ethyl)piperazin-1-yl]ethoxy]-2-methyl-phenyl]pyridine-2-carboxylate (1.3 g, 1.64 mmol, 1 equiv.) in THE (2 mL) was added LiOH·H₂O (1 M, 4.93 mL, 3 equiv.). Then, the mixture was stirred at 25° C. for 2 hours. The reaction mixture was filtered and concentrated under reduced pressure to give a residue. The residue was purified by reverse-phase HPLC. The compound 2-[4-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]ethyl]piperazin-1-yl]acetic acid (250 mg, 327.6 μmol, 19.9% yield) was obtained as a white solid.

MS (ESI) m/z: 763.3 [M+H]⁺

### Step E. Procedure for Preparation of tert-butyl 6-[8-(1,3-benzothiazol-2-ylcarbamoyl)-3,4-dihydro-1H-isoquinolin-2-yl]-3-[3-[2-[4-[2-[[3-(2,6-dioxo-3-piperidyl)-1-methyl-indazol-6-yl]amino]-2-oxo-ethyl]piperazin-1-yl]ethoxy]-2-methyl-phenyl]pyridine-2-carboxylate

To a solution of 2-[4-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]ethyl]piperazin-1-yl]acetic acid (140 mg, 183.51 umol, 1 equiv.) and 3-(6-amino-1-methyl-indazol-3-yl)piperidine-2,6-dione (56.87 mg, 220.21 umol, 1.2 equiv.) in DMF (0.1 mL) was added DIPEA (71.15 mg, 550.52 μmol, 95.89 μL, 3 equiv.) and HATU (83.73 mg, 220.21 umol, 1.2 equiv.). Then, the mixture was stirred at 25° C. for 15 hours. The reaction mixture was added to water (2 mL) slowly, and a grey solid precipitated. The combined solution was filtered, dissolved in DCM (10 mL), and concentrated under reduced pressure to give a residue. The compound tert-butyl 6-[8-(1,3-benzothiazol-2-ylcarbamoyl)-3,4-dihydro-1H-isoquinolin-2-yl]-3-[3-[2-[4-[2-[[3-(2,6-dioxo-3-piperidyl)-1-methyl-indazol-6-yl]amino]-2-oxo-ethyl]piperazin-1-yl]ethoxy]-2-methyl-phenyl]pyridine-2-carboxylate (220 mg, crude) was obtained as a brown solid.

MS (ESI) m/z: 1003.5 [M+H]⁺

### Step F. Procedure for Preparation of 6-[8-(1,3-benzothiazol-2-ylcarbamoyl)-3,4-dihydro-1H-isoquinolin-2-yl]-3-[3-[2-[4-[2-[[3-(2,6-dioxo-3-piperidyl)-1-methyl-indazol-6-yl]amino]-2-oxo-ethyl]piperazin-1-yl]ethoxy]-2-methyl-phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[3-[2-[4-[2-[[3-(2,6-dioxo-3-piperidyl)-1-methyl-indazol-6-yl]amino]-2-oxo-ethyl]piperazin-1-yl]ethoxy]-2-methyl-phenyl]pyridine-2-carboxylate (113.46 mg, 113.10 umol, 1 equiv.) and TFA (12.90 mg, 113.10 umol, 8.37 μL, 1 equiv.) in DCM (1 mL) was stirred at 25° C. for 4 hours. The reaction mixture was concentrated under reduced pressure to give a residue. The residue was purified by prep-HPLC. The compound 6-[8-(1,3-benzothiazol-2-ylcarbamoyl)-3,4-dihydro-1H-isoquinolin-2-yl]-3-[3-[2-[4-[2-[[3-(2,6-dioxo-3-piperidyl)-1-methyl-indazol-6-yl]amino]-2-oxo-ethyl]piperazin-1-yl]ethoxy]-2-methyl-phenyl]pyridine-2-carboxylic acid (53 mg, 56.0 μmol, 49.5% yield) was obtained as a yellow solid.

MS (ESI) m/z: 947.3 [M+H]⁺

¹H NMR (400 MHz, DMSO-d₆) δ=12.99-12.68 (m, 1H), 10.81 (s, 1H), 9.85-9.73 (m, 1H), 8.03 (d, J=8.0 Hz, 1H), 7.79 (d, J=8.0 Hz, 1H), 7.63 (d, J=7.2 Hz, 1H), 7.55 (d, J=8.4 Hz, 2H), 7.49-7.39 (m, 4H), 7.38-7.33 (m, 2H), 7.16 (d, J=7.6 Hz, 3H), 6.98 (d, J=8.88.9 Hz, 2H), 6.69 (d, J=5.2 Hz, 1H), 4.98 (s, 2H), 4.44-4.23 (m, 2H), 3.92 (t, J=6.0 Hz, 2H), 3.80 (dd, J=4.4, 11.2 Hz, 1H), 3.62-3.54 (m, 2H), 3.28 (s, 2H), 3.04-3.02 (m, 2H), 2.72-2.61 (m, 4H), 2.54 (s, 4H), 2.24-2.10 (m, 2H), 2.09-1.96 (m, 2H), 1.94 (s, 3H)

### Example 120. Preparation of Compound 104

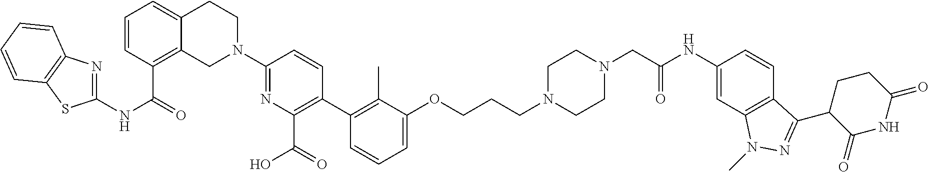 [SMILES: Cc1c(OCCCN2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[4-[2-[[3-(2,6-dioxo-3-piperidyl)-1-methyl-indazol-6-yl]amino]-2-oxo-ethyl]piperazin-1-yl]propoxy]-2-methyl-phenyl]pyridine-2-carboxylic acid

### Step A. Procedure for Preparation of 1-bromo-3-(3-bromopropoxy)-2-methyl-benzene

A mixture of 3-bromo-2-methyl-phenol (2 g, 10.69 mmol, 1 equiv.), 1,3-dibromopropane (10.79 g, 53.47 mmol, 5.45 mL, 5 equiv.) and Cs₂CO₃ (10.45 g, 32.08 mmol, 3 equiv.) in CH₃CN (25 mL) was stirred at 25° C. for 5 hours. The reaction mixture was filtered and concentrated under reduced pressure to give a residue. The residue was purified by flash silica gel chromatography (Eluent of 0˜1% ethyl acetate/petroleum ether). The compound 1-bromo-3-(3-bromopropoxy)-2-methyl-benzene (2.4 g, 7.0 mmol, 65.8% yield, 90% purity) was obtained as a white solid.

### Step B. Procedure for Preparation of ethyl 2-[4-[3-(3-bromo-2-methyl-phenoxy)propyl]piperazin-1-yl]acetate

A mixture of 1-bromo-3-(3-bromopropoxy)-2-methyl-benzene (2.15 g, 6.97 mmol, 1 equiv.), ethyl 2-piperazin-1-ylacetate (1.2 g, 6.97 mmol, 1 equiv.) and DIPEA (3.60 g, 27.87 mmol, 4.85 mL, 4 equiv.) in DMF (25 mL) was stirred at 60° C. for 3 hours. The reaction mixture was filtered and concentrated under reduced pressure to give a residue. The residue was purified by flash silica gel chromatography (Eluent of 0˜10% ethyl acetate/petroleum ether) to give ethyl 2-[4-[3-(3-bromo-2-methyl-phenoxy)propyl]piperazin-1-yl]acetate (1.3 g, 3.1 mmol, 46.0% yield, 95% purity) as a white solid.

MS (ESI) m/z: 401.1 [M+H]⁺ (⁸⁰Br).

### Step C. Procedure for Preparation of tert-butyl 6-[8-(1,3-benzothiazol-2-ylcarbamoyl)-3,4-dihydro-1H-isoquinolin-2-yl]-3-[3-[3-[4-(2-ethoxy-2-oxo-ethyl)piperazin-1-yl]propoxy]-2-methyl-phenyl]pyridine-2-carboxylate

A mixture of ethyl 2-[4-[3-(3-bromo-2-methyl-phenoxy)propyl]piperazin-1-yl]acetate (380 mg, 951.61 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (816.07 mg, 1.33 mmol, 1.4 equiv.), [2-(2-aminophenyl)phenyl]palladium(1+); bis(1-adamantyl)-butyl-phosphane; methanesulfonate (138.61 mg, 190.32 μmol, 0.2 equiv.), and K₂CO₃ (1.5 M, 951.61 μL, 1.5 equiv.) in dioxane (3 mL) was degassed and purged with N₂ three times, and then the mixture was stirred at 100° C. for 1 hour under microwave. The reaction mixture was filtered and concentrated under reduced pressure to give a residue. The residue was purified by flash silica gel chromatography (Eluent of 0˜10% DCM/MeOH) to give tert-butyl 6-[8-(1,3-benzothiazol-2-ylcarbamoyl)-3,4-dihydro-1H-isoquinolin-2-yl]-3-[3-[3-[4-(2-ethoxy-2-oxo-ethyl)piperazin-1-yl]propoxy]-2-methyl-phenyl]pyridine-2-carboxylate (546 mg, 610.44 μmol, 64.15% yield, 90% purity) as a white solid.

MS (ESI) m/z: 805.5 [M+H]⁺.

¹H NMR (400 MHz, CD₃OD) δ=7.90 (d, J=8.0 Hz, 1H), 7.75 (d, J=8.0 Hz, 1H), 7.58 (d, J=7.6 Hz, 1H), 7.42 (d, J=8.4 Hz, 1H), 7.37-7.34 (m, 1H), 7.14-7.06 (m, 2H), 7.02 (t, J=8.0 Hz, 1H), 6.94 (d, J=8.8 Hz, 1H), 6.89 (dd, J=4.4, 8.0 Hz, 2H), 6.63 (d, J=7.6 Hz, 1H), 5.02 (s, 2H), 4.17 (dq, J=2.8, 7.2 Hz, 4H), 3.98 (t, J=6.0 Hz, 2H), 3.25 (d, J=3.6 Hz, 3H), 3.08 (t, J=6.0 Hz, 2H), 2.67-2.63 (m, 10H), 2.28 (s, 2H), 1.93 (s, 3H), 1.80 (s, 2H), 1.08 (s, 9H)

### Step D. Procedure for Preparation of 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]piperazin-1-yl]acetic acid

A mixture of tert-butyl 6-[8-(1,3-benzothiazol-2-ylcarbamoyl)-3,4-dihydro-1H-isoquinolin-2-yl]-3-[3-[3-[4-(2-ethoxy-2-oxo-ethyl)piperazin-1-yl]propoxy]-2-methyl-phenyl]pyridine-2-carboxylate (230 mg, 285.72 μmol, 1 equiv.) and LiOH·H2O (35.97 mg, 857.15 μmol, 3 equiv.) in THE (1.2 mL) and H₂O (0.4 mL) was stirred at 25° C. for 1.5 hour. The reaction mixture was concentrated and redissolved by H₂O (2 mL), then 1M HCl was added to adjust the pH to 2, the residue was filtered and dried. The residue was used for next step without further purification. The compound 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]piperazin-1-yl]acetic acid (206 mg, 225.3 μmol, 78.8% yield) was obtained as a white solid.

### Step E. Procedure for Preparation of tert-butyl 6-[8-(1,3-benzothiazol-2-ylcarbamoyl)-3,4-dihydro-1H-isoquinolin-2-yl]-3-[3-[3-[4-[2-[[3-(2,6-dioxo-3-piperidyl)-1-methyl-indazol-6-yl]amino]-2-oxo-ethyl]piperazin-1-yl]propoxy]-2-methyl-phenyl]pyridine-2-carboxylate

A mixture of 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]piperazin-1-yl]acetic acid (150 mg, 193.06 μmol, 1 equiv.), 3-(6-amino-1-methyl-indazol-3-yl)piperidine-2,6-dione (59.84 mg, 231.68 μmol, 1.2 equiv.), HATU (73.41 mg, 193.06 μmol, 1 equiv.), and DIPEA (74.86 mg, 579.19 μmol, 100.88 μL, 3 equiv.) in DMF (2 mL) was stirred at 25° C. for 10 hours. The reaction mixture was quenched by addition H₂O (2 mL), and then extracted with ethyl acetate 6 mL (2 mL*3). The combined organic layers were washed with saturated sodium chloride (3 mL), dried over anhydrous sodium sulfate, filtered, and concentrated under reduced pressure to give a residue. The reaction mixture was used for next step without other purification. The compound tert-butyl 6-[8-(1,3-benzothiazol-2-ylcarbamoyl)-3,4-dihydro-1H-isoquinolin-2-yl]-3-[3-[3-[4-[2-[[3-(2,6-dioxo-3-piperidyl)-1-methyl-indazol-6-yl]amino]-2-oxo-ethyl]piperazin-1-yl]propoxy]-2-methyl-phenyl]pyridine-2-carboxylate (150 mg, 125.3 μmol, 64.9% yield) was obtained as a white solid.

MS (ESI) m/z: 1017.6 [M+H]⁺.

### Step F. Procedure for Preparation of 6-[8-(1,3-benzothiazol-2-ylcarbamoyl)-3,4-dihydro-1H-isoquinolin-2-yl]-3-[3-[3-[4-[2-[[3-(2,6-dioxo-3-piperidyl)-1-methyl-indazol-6-yl]amino]-2-oxo-ethyl]piperazin-1-yl]propoxy]-2-methyl-phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[3-[3-[4-[2-[[3-(2,6-dioxo-3-piperidyl)-1-methyl-indazol-6-yl]amino]-2-oxo-ethyl]piperazin-1-yl]propoxy]-2-methyl-phenyl]pyridine-2-carboxylate (120 mg, 117.97 μmol, 1 equiv.) and TFA (1.54 g, 13.51 mmol, 1 mL, 114.49 equiv.) in DCM (1 mL) was stirred at 40° C. for 3 hours. The reaction mixture was filtered and concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[3-[4-[2-[[3-(2,6-dioxo-3-piperidyl)-1-methyl-indazol-6-yl]amino]-2-oxo-ethyl]piperazin-1-yl]propoxy]-2-methyl-phenyl]pyridine-2-carboxylic acid (42.32 mg, 43.3 μmol, 36.7% yield, 98.3% purity) was obtained as a white solid.

MS (ESI) m/z: 961.2 [M+H]⁺.

¹H NMR (400 MHz, d₆-DMSO) δ=10.88 (s, 1H), 9.85 (s, 1H), 8.15 (s, 1H), 8.07-8.00 (m, 2H), 7.79 (d, J=8.0 Hz, 1H), 7.63 (d, J=8.8 Hz, 2H), 7.50-7.41 (m, 3H), 7.40-7.31 (m, 2H), 7.20 (dd, J=8.8, 1.6 Hz, 1H), 7.13-7.06 (m, 1H), 6.96 (d, J=8.8 Hz, 1H), 6.88 (d, J=8.4 Hz, 1H), 6.64 (d, J=7.6 Hz, 1H), 4.98 (s, 2H), 4.32 (dd, J=9.6, 5.2 Hz, 1H), 4.00 (t, J=6.0 Hz, 2H), 3.92 (s, 5H), 3.16 (s, 3H), 3.02 (t, J=5.6 Hz, 2H), 2.65-2.52 (m, 10H), 2.41-2.28 (m, 2H), 2.21-2.14 (m, 1H), 1.95-1.88 (m, 5H).

### Example 121. Preparation of Compound 105

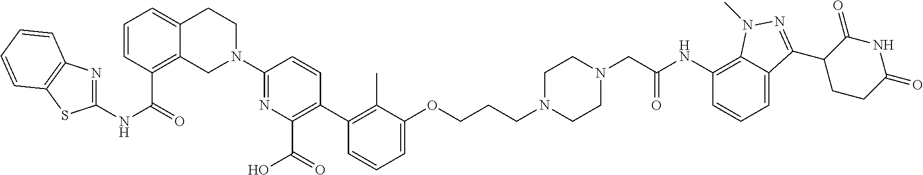 [SMILES: Cc1c(OCCCN2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[4-[2-[[3-(2,6-dioxo-3-piperidyl)-1-methyl-indazol-7-yl]amino]-2-oxo-ethyl]piperazin-1-yl]propoxy]-2-methyl-phenyl]pyridine-2-carboxylic acid

### Step A. Procedure for Preparation of 7-bromo-3-iodo-1-methyl-1H-indazole

To a solution of compound 7-bromo-3-iodo-1H-indazole (298 g, 849 mmol, 1.00 eq) in DMF (1.79 L) was added Cs₂CO₃ (415 g, 1.27 mol, 1.50 eq) at 0° C., then the suspension was stirred at 0° C. for 1 h. Then CH₃I (181 g, 1.27 mol, 79 mL, 1.50 eq) was added dropwise to the reaction at 0° C., after addition, the suspension was stirred at 25° C. for 2 h. The reaction suspension was poured into water (8.4 L) and stirred for 10 min. The aqueous phase was extracted with ethyl acetate (800 mL, 400 mL). The combined organic phase was washed with brine (400 mL), dried with anhydrous Na₂SO₄, filtered, and concentrated in vacuum. The residue was purified by column chromatography (SiO₂, petroleum ether/ethyl acetate=100/1 to 0/1) to give compound 7-bromo-3-iodo-1-methyl-1H-indazole (160 g, 52.3% yield) as an off-white solid.

MS (ESI) m/z: 336.8 [M+H]⁺

¹H NMR: (400 MHz, DMSO-d₆) δ 7.73 (d, J=6.4 Hz, 1H), 7.46 (d, J=8.0 Hz, 1H), 7.12 (t, J=8.0 Hz, 1H), 4.34 (s, 1H).

### Step B. Procedure for Preparation of 3-(2,6-bis(benzyloxy)pyridin-3-yl)-7-bromo-1-methyl-1H-indazole

To a solution of compound 7-bromo-3-iodo-1-methyl-1H-indazole (140 g, 415 mmol, 1.00 eq) in dioxane (980 mL) and H₂O (490 mL) was added compound (2,6-bis(benzyloxy)pyridin-3-yl)boronic acid (139 g, 415 mmol, 1.00 eq), K₃PO₄ (220 g, 1.04 mol, 2.50 eq) and Pd(PPh₃)₄ (14.4 g, 12.5 mmol, 0.03 eq). Then the suspension was purged with N₂ for three times and stirred at 90° C. for 24 h. The reaction was cooled to 20° C. and filtered by a pad of celite. The filtrate was poured into water (900 mL) and the aqueous phase was extracted with ethyl acetate (900 mL, 450 mL). The combined organic phase was washed with brine (400 mL), dried with anhydrous Na₂SO₄, filtered, and concentrated in vacuum. The residue was purified by column chromatography (SiO2, petroleum ether/ethyl acetate=100/1 to 0/1) to give compound 3-(2,6-bis(benzyloxy)pyridin-3-yl)-7-bromo-1-methyl-1H-indazole (120 g, 240 mmol, 57.7% yield) as an off-white solid.

MS (ESI) m/z: 500.1 [M+H]⁺

¹H NMR: (400 MHz, DMSO-d₆) δ 7.86 (d, J=8.0 Hz, 1H), 7.46-7.59 (m, 2H), 7.35-7.39 (m, 9H), 7.27-7.33 (m, 7H), 6.93 (t, J=8.0 Hz, 1H), 6.60 (d, J=8.0 Hz, 1H), 5.43 (d, J=1.2 Hz, 4H), 4.35 (s, 3H).

### Step C. Procedure for Preparation of N-benzyl-3-(2,6-bis(benzyloxy)pyridin-3-yl)-1-methyl-1H-indazol-7-amine

To a solution of compound 3-(2,6-bis(benzyloxy)pyridin-3-yl)-7-bromo-1-methyl-1H-indazole (120 g, 240 mmol, 1.00 eq), phenylmethanamine (38.6 g, 360 mmol, 39.2 mL, 1.50 eq), Cs₂CO₃ (156 g, 479 mmol, 2.00 eq), BINAP (10.5 g, 16.8 mmol, 0.07 eq) and Pd₂(dba)₃ (4.39 g, 4.80 mmol, 0.02 eq) in dioxane (1.2 L) was degassed and purged with N₂ three times, and then the mixture was stirred at 110° C. for 16 h under N₂ atmosphere. The reaction suspension was filtered by a pad of celite and the filter cake was washed with THE (300 mL). The filtrate was concentrated under vacuum. The residue was purified by column chromatography (SiO₂, petroleum ether/ethyl acetate=100/1 to 0/1). The crude was triturated with EtOH (250 mL) at 20° C. for 1 h. The solid was collected by filtration and dried under vacuum to give compound N-benzyl-3-(2,6-bis(benzyloxy)pyridin-3-yl)-1-methyl-1H-indazol-7-amine (110 g, 209 mmol, 87.1% yield) as a yellow solid.

MS (ESI) m/z: 527.2 [M+H]⁺

¹H NMR: (400 MHz, DMSO-d₆) δ 7.81 (d, J=8.0 Hz, 1H), 7.34-7.46 (m, 4H), 7.26-7.33 (m, 12H), 6.89 (d, J=8.0 Hz, 1H)), 6.74 (t, J=7.2 Hz, 1H), 6.57 (d, J=8.0 Hz, 1H), 6.30 (d, J=7.2 Hz, 1H), 6.09 (t, J=8.0 Hz, 1H), 5.40 (s, 4H), 4.40 (s, 5H).

### Step D. Procedure for Preparation of 3-(7-amino-1-methyl-1H-indazol-3-yl)piperidine-2,6-dione

To a solution of N-benzyl-3-(2,6-bis(benzyloxy)pyridin-3-yl)-1-methyl-1H-indazol-7-amine (60 g, 114 mmol, 1.00 eq) and AcOH (6.84 g, 114 mmol, 6.52 mL, 1.00 eq) in THE (300 mL) and EtOH (300 mL) was added Pd/C (15.0 g, 10% purity) and Pd(OH)₂ (15.0 g, 21.4 mmol, 20% purity) then the suspension was stirred at 50° C. under 50 psi (H₂) for 12 h. The two reactions were combined for work-up. The mixture was filtered, and the filter cake was washed with THE (4.0 L). The filtrate was concentrated under reduced pressure to give a residue. The crude product was triturated with EtOAC/MTBE=1/1 (150 mL) at 25° C. for 5 h. The solid was collected by filtration and dried under vacuum to give 3-(7-amino-1-methyl-1H-indazol-3-yl)piperidine-2,6-dione (51.0 g, 83.9% yield) as a off-white solid.

MS (ESI) m/z: 259.2 [M+H]⁺

¹H NMR: (400 MHz, DMSO-d₆) δ 10.85 (s, 1H), 6.91 (d, J=8.0 Hz, 1H), 6.80 (t, J=7.6 Hz, 1H), 6.55 (dd, J=7.2 Hz, 1H), 5.18 (s, 2H), 4.24 (dd, J=4.4, 5.2 Hz, 1H), 4.20 (s, 3H), 3.58-3.61 (m, 1H), 2.58-2.64 (m, 2H), 2.26-2.29 (m, 1H), 2.15-2.16 (m, 1H)

### Step E. Procedure for Preparation of tert-butyl 6-[8-(1,3-benzothiazol-2-ylcarbamoyl)-3,4-dihydro-1H-isoquinolin-2-yl]-3-[3-[3-[4-[2-[[3-(2,6-dioxo-3-piperidyl)-1-methyl-indazol-7-yl]amino]-2-oxo-ethyl]piperazin-1-yl]propoxy]-2-methyl-phenyl]pyridine-2-carboxylate

A mixture of 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]piperazin-1-yl]acetic acid (200 mg, 257.42 μmol, 1 equiv.), 3-(7-amino-1-methyl-1H-indazol-3-yl)piperidine-2,6-dione (73.13 mg, 283.16 μmol, 1.1 equiv.), HATU (97.88 mg, 257.42 μmol, 1 equiv.), and DIPEA (99.81 mg, 772.26 μmol, 134.51 μL, 3 equiv.) in DMF (2 mL) was stirred at 25° C. for 10 hours. The reaction mixture was quenched by addition of H₂O (2 mL), and then extracted with ethyl acetate 6 mL (2 mL×3). The combined organic layers were washed with saturated sodium chloride (3 mL), dried over anhydrous sodium sulfate, filtered, and concentrated under reduced pressure to give a residue. The residue was used for next step without other purification. The compound tert-butyl 6-[8-(1,3-benzothiazol-2-ylcarbamoyl)-3,4-dihydro-1H-isoquinolin-2-yl]-3-[3-[3-[4-[2-[[3-(2,6-dioxo-3-piperidyl)-1-methyl-indazol-7-yl]amino]-2-oxo-ethyl]piperazin-1-yl]propoxy]-2-methyl-phenyl]pyridine-2-carboxylate (130 mg, 108.6 μmol, 42.2% yield, 85% purity) was obtained as a white solid.

MS (ESI) m/z: 1017.6 [M+H]⁺.

### Step F. Procedure for Preparation of 6-[8-(1,3-benzothiazol-2-ylcarbamoyl)-3,4-dihydro-1H-isoquinolin-2-yl]-3-[3-[3-[4-[2-[[3-(2,6-dioxo-3-piperidyl)-1-methyl-indazol-7-yl]amino]-2-oxo-ethyl]piperazin-1-yl]propoxy]-2-methyl-phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[3-[3-[4-[2-[[3-(2,6-dioxo-3-piperidyl)-1-methyl-indazol-7-yl]amino]-2-oxo-ethyl]piperazin-1-yl]propoxy]-2-methyl-phenyl]pyridine-2-carboxylate (130 mg, 127.80 μmol, 1 equiv.) and TFA (14.57 mg, 127.80 μmol, 9.46 μL, 1 equiv.) in DCM (2 mL) was stirred at 40° C. for 2 hour. The reaction mixture was filtered and concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[3-[4-[2-[[3-(2,6-dioxo-3-piperidyl)-1-methyl-indazol-7-yl]amino]-2-oxo-ethyl]piperazin-1-yl]propoxy]-2-methyl-phenyl]pyridine-2-carboxylic acid (18.8 mg, 18.6 μmol, 14.6% yield, 95.5% purity) as a white solid.

MS (ESI) m/z: 961.2 [M+H]⁺.

¹H NMR (400 MHz, d₆-DMSO) δ=12.28-11.84 (m, 1H), 10.95-10.80 (m, 2H), 8.03 (d, J=7.6 Hz, 1H), 7.79 (d, J=8.0 Hz, 1H), 7.64 (dd, J=14.0, 7.6 Hz, 2H), 7.49-7.25 (m, 6H), 7.21 (d, J=7.2 Hz, 1H), 7.12 (t, J=7.6 Hz, 2H), 6.99 (d, J=8.8 Hz, 1H), 6.91 (d, J=8.4 Hz, 1H), 6.66 (d, J=7.6 Hz, 1H), 4.98 (s, 2H), 4.50-4.35 (m, 2H), 4.16-4.03 (m, 6H), 3.92 (t, J=5.6 Hz, 2H), 3.79-3.65 (m, 4H), 3.56-3.25 (m, 5H), 3.03 (t, J=5.6 Hz, 2H), 2.71-2.60 (m, 2H), 2.44-2.10 (m, 5H), 1.93 (s, 3H).

### Example 122. Preparation of Compound 107

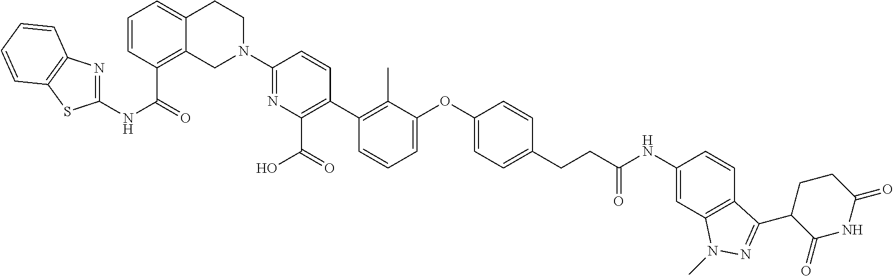 [SMILES: Cc1c(Oc2ccc(CCC(=O)Nc3ccc4c(C5CCC(=O)NC5=O)nn(C)c4c3)cc2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[4-[3-[[3-(2,6-dioxo-3-piperidyl)-1-methyl-indazol-6-yl]amino]-3-oxo-propyl]phenoxy]-2-methyl-phenyl]pyridine-2-carboxylic acid

### Step A. Procedure for Preparation of tert-butyl 6-[8-(1, 3-benzothiazol-2-ylcarbamoyl)-3, 4-dihydro-1H-isoquinolin-2-yl]-3-[3-[4-[3-[[3-(2, 6-dioxo-3-piperidyl)-1-methyl-indazol-6-yl]amino]-3-oxo-propyl]phenoxy]-2-methyl-phenyl]pyridine-2-carboxylate

To a solution of 3-[4-[3-[6-[8-(1, 3-benzothiazol-2-ylcarbamoyl)-3, 4-dihydro-1H-isoquinolin-2-yl]-2-tert-butoxycarbonyl-3-pyridyl]-2-methyl-phenoxy]phenyl]propanoic acid (200 mg, 269.95 mol, 1.1 equiv.) in DMF (2 mL) was added 3-(6-amino-1-methyl-indazol-3-yl) piperidine-2, 6-dione (63.38 mg, 245.41 μmol, 1 equiv.), HATU (102.64 mg, 269.95 μmol, 1.1 equiv.), and DIPEA (95.15 mg, 736.24 μmol, 128.24 μL, 3 equiv.) at 25° C. The reaction mixture was stirred at 25° C. for 16 hours. The reaction mixture was diluted with water (10 mL) and filtered to give a residue. The compound tert-butyl 6-[8-(1, 3-benzothiazol-2-ylcarbamoyl)-3, 4-dihydro-1H-isoquinolin-2-yl]-3-[3-[4-[3-[[3-(2, 6-dioxo-3-piperidyl)-1-methyl-indazol-6-yl]amino]-3-oxo-propyl]phenoxy]-2-methyl-phenyl]pyridine-2-carboxylate (240 mg, crude) was obtained as a yellow solid.

MS (ESI) m/z: 981.5 [M+H]⁺.

### Step B. Procedure for Preparation of 6-[8-(1,3-benzothiazol-2-ylcarbamoyl)-3,4-dihydro-1H-isoquinolin-2-yl]-3-[3-[4-[3-[[3-(2,6-dioxo-3-piperidyl)-1-methyl-indazol-6-yl]amino]-3-oxo-propyl]phenoxy]-2-methyl-phenyl]pyridine-2-carboxylic acid

A solution of tert-butyl 6-[8-(1, 3-benzothiazol-2-ylcarbamoyl)-3, 4-dihydro-1H-isoquinolin-2-yl]-3-[3-[4-[3-[[3-(2, 6-dioxo-3-piperidyl)-1-methyl-indazol-6-yl]amino]-3-oxo-propyl]phenoxy]-2-methyl-phenyl]pyridine-2-carboxylate (240 mg, 244.62 μmol, 1 equiv.) in DCM (1 mL) and TFA (1.5 mL) was stirred at 40° C. for 2 hour. The reaction mixture was concentrated under reduced pressure to give a residue. The residue was purified by prep-HPLC. The compound 6-[8-(1,3-benzothiazol-2-ylcarbamoyl)-3,4-dihydro-1H-isoquinolin-2-yl]-3-[3-[4-[3-[[3-(2,6-dioxo-3-piperidyl)-1-methyl-indazol-6-yl]amino]-3-oxo-propyl]phenoxy]-2-methyl-phenyl]pyridine-2-carboxylic acid (40.5 mg, 41.4 μmol, 16.9% yield, 99.2% purity) was obtained as a yellow solid.

MS (ESI) m/z: 925.6 [M+H]⁺.

¹H NMR (400 MHz, DMSO-d₆) δ=10.87 (s, 1H), 10.09 (s, 1H), 8.08-8.00 (m, 2H), 7.79 (d, J=8.0 Hz, 1H), 7.66-7.31 (m, 8H), 7.23 (d, J=8.4 Hz, 2H), 7.16 (t, J=7.6 Hz, 1H), 7.08-7.04 (m, 1H), 7.00 (d, J=8.8 Hz, 1H), 6.88 (d, J=7.2 Hz, 1H), 6.82 (d, J=8.4 Hz, 3H), 4.99 (s, 2H), 4.31 (dd, J=5.2, 9.6 Hz, 1H), 3.95-3.91 (m, 2H), 3.91 (s, 3H), 3.03 (t, J=5.6 Hz, 2H), 2.94-2.87 (m, 2H), 2.69-2.61 (m, 4H), 2.38-2.28 (m, 1H), 2.20-2.17 (m, 1H), 1.89 (s, 3H)

### Example 123. Preparation of Compound 109

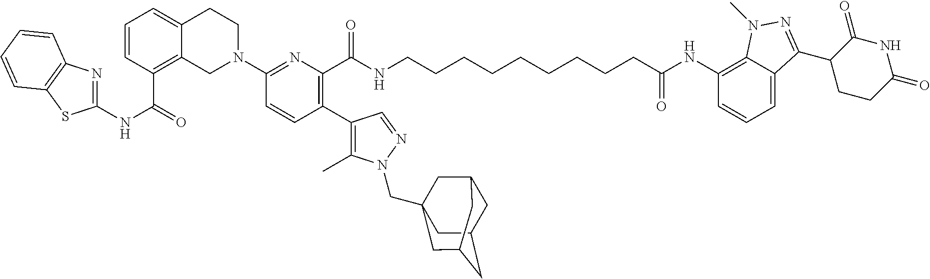 [SMILES: Cc1c(-c2ccc(N3CCc4cccc(C(=O)Nc5nc6ccccc6s5)c4C3)nc2C(=O)NCCCCCCCCCC(=O)Nc2cccc3c(C4CCC(=O)NC4=O)nn(C)c23)cnn1CC12CC3CC(CC(C3)C1)C2]

### 2-(5-(1-((3R, 5R, 7R)-adamantan-1-ylmethyl)-5-methyl-1H-pyrazol-4-yl)-6-((10-((3-(2, 6-dioxopiperidin-3-yl)-1-methyl-1H-indazol-7-yl) amino)-10-oxodecyl) carbamoyl) pyridin-2-yl)-N-(benzo[d]thiazol-2-yl)-1, 2, 3, 4-tetrahydroisoquinoline-8-carboxamide

### Step A. Procedure for Preparation of methyl 10-aminodecanoate

To a solution of 10-aminodecanoic acid (1 g, 5.34 mmol, 1 equiv.) in MeOH (10 mL) was added SOCl₂ (952.89 mg, 8.01 mmol, 581.03 μL, 1.5 equiv.) at 25° C. The reaction mixture was stirred at 70° C. for 16 hours. The reaction mixture was concentrated under reduced pressure to give methyl 10-aminodecanoate (1.22 g, crude) as an off-white solid.

¹H NMR (400 MHz, CD₃OD) δ=3.64 (s, 3H), 2.91 (t, J=7.6 Hz, 2H), 2.32 (t, J=7.6 Hz, 2H), 1.58-1.68 (m, 4H), 1.40-1.32 (m, 10H).

### Step B. Procedure for Preparation of methyl 10-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)decanoate

To a solution of 3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinic acid (1 g, 1.52 mmol, 1 equiv.) in DMF (10 mL) was added methyl 10-aminodecanoate (360.90 mg, 1.52 mmol, 1 equiv., HCl), HATU (577.15 mg, 1.52 mmol, 1 equiv.), and DIPEA (588.53 mg, 4.55 mmol, 793.17 μL, 3 equiv.) at 25° C. The reaction mixture was stirred at 25° C. for 1.5 hours. The reaction mixture was diluted with H₂O (10 mL) and extracted with EtOAc (30 mL×3). The combined organic layers were washed with NaHCO₃ (10 mL×3), brine (10 mL×3), dried over anhydrous Na₂SO₄, filtered, and concentrated under reduced pressure to give a residue. The residue was purified by flash silica gel chromatography (Eluent of 0˜8% MeOH/DCM) to give methyl 10-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)decanoate (861 mg, 989.7 μmol, 65.2% yield, 96.8% purity) as a yellow oil.

MS (ESI) m/z: 842.9 [M+H]⁺.

### Step C. Procedure for Preparation of 10-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)decanoic acid

To a solution of methyl 10-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)decanoate (831 mg, 986.82 μmol, 1 equiv.) in THE (1 mL) and H₂O (0.1 mL) was added LiOH·H₂O (124.23 mg, 2.96 mmol, 3 equiv.) at 25° C. The reaction mixture was stirred at 40° C. for 20 hours. The reaction mixture was concentrated under reduced pressure to give a residue. The residue was treated with water (4 mL). The pH was adjusted to about 3 by progressively adding 1 M HCl. The mixture was filtered and concentrated under reduced pressure to give 10-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)decanoic acid (670 mg, 778.6 μmol, 78.9% yield, 96.2% purity) as a yellow solid.

MS (ESI) m/z: 828.8 [M+H]⁺.

¹H NMR (400 MHz, CD₃OD) δ=7.92 (d, J=7.6 Hz, 1H), 7.78 (d, J=8.0 Hz, 1H), 7.58 (dd, J=1.2, 7.6 Hz, 1H), 7.49-7.44 (m, 2H), 7.42-7.39 (m, 1H), 7.39-7.37 (m, 1H), 7.36 (d, J=2.4 Hz, 1H), 7.33 (s, 1H), 7.03 (d, J=8.8 Hz, 1H), 5.14 (s, 2H), 3.93 (t, J=6.0 Hz, 2H), 3.74 (s, 2H), 3.16 (t, J=7.2 Hz, 2H), 3.08 (t, J=6.0 Hz, 2H), 2.23 (t, J=7.2 Hz, 2H), 2.10 (s, 3H), 1.97 (s, 3H), 1.77-1.65 (m, 6H), 1.61 (d, J=2.0 Hz, 6H), 1.57-1.51 (m, 2H), 1.42 (s, 2H), 1.33-1.23 (m, 4H), 1.16 (d, J=3.2 Hz, 6H)

### Step D. Procedure for Preparation of 2-(5-(1-((3R, 5R, 7R)-adamantan-1-ylmethyl)-5-methyl-1H-pyrazol-4-yl)-6-((10-((3-(2, 6-dioxopiperidin-3-yl)-1-methyl-1H-indazol-7-yl) amino)-10-oxodecyl) carbamoyl) pyridin-2-yl)-N-(benzo[d]thiazol-2-yl)-1, 2, 3, 4-tetrahydroisoquinoline-8-carboxamide

To a solution of 10-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)decanoic acid (100.00 mg, 120.76 μmol, 1 equiv.) in DMF (1 mL) was added 3-(7-amino-1-methyl-1H-indazol-3-yl)piperidine-2,6-dione (31.19 mg, 120.76 μmol, 1 equiv.), HATU (45.92 mg, 120.76 μmol, 1 equiv.) and DIPEA (46.82 mg, 362.29 μmol, 63.10 μL, 3 equiv.) at 25° C. The reaction mixture was stirred at 25° C. for 16 hours. The reaction mixture was quenched by addition water 1 mL at 25° C., and filtered to give a residue. The residue was purified by prep-HPLC to give compound 2-(5-(1-((3R, 5R, 7R)-adamantan-1-ylmethyl)-5-methyl-1H-pyrazol-4-yl)-6-((10-((3-(2, 6-dioxopiperidin-3-yl)-1-methyl-1H-indazol-7-yl) amino)-10-oxodecyl) carbamoyl) pyridin-2-yl)-N-(benzo[d]thiazol-2-yl)-1, 2, 3, 4-tetrahydroisoquinoline-8-carboxamide (29.2 mg, 25.6 μmol, 21.2% yield, 93.5% purity) was obtained as a yellow solid.

MS (ESI) m/z: 1068.3 [M+H]⁺

¹H NMR (400 MHz, DMSO-d₆) δ=12.85-12.80 (m, 1H), 10.91 (s, 1H), 9.82 (s, 1H), 8.06 (s, 1H), 8.01 (d, J=8.0 Hz, 1H), 7.78 (d, J=8.0 Hz, 1H), 7.62-7.57 (m, 2H), 7.49-7.43 (m, 2H), 7.43-7.39 (m, 1H), 7.37-7.32 (m, 2H), 7.23 (s, 1H), 7.11-7.04 (m, 2H), 6.95 (d, J=8.8 Hz, 1H), 5.00 (s, 2H), 4.37 (dd, J=5.2, 10.0 Hz, 1H), 4.03 (s, 3H), 3.88 (t, J=6.0 Hz, 2H), 3.67 (s, 2H), 3.01 (d, J=4.4 Hz, 4H), 2.69-2.56 (m, 2H), 2.40-2.32 (m, 3H), 2.20-2.13 (m, 1H), 1.91 (s, 4H), 1.66-1.59 (m, 5H), 1.56 (s, 2H), 1.51 (s, 7H), 1.33-1.20 (m, 7H), 1.20-1.07 (m, 7H)

### Example 124. Preparation of Compound 110

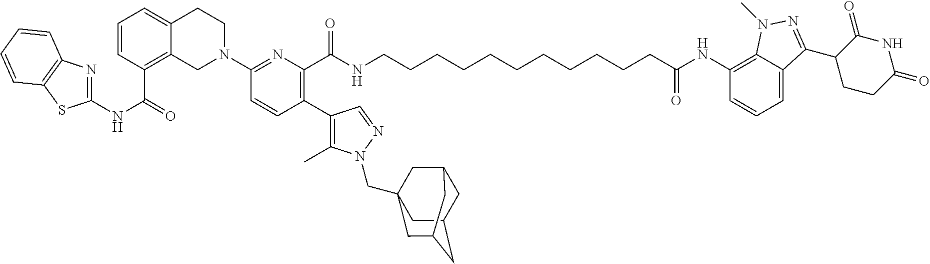 [SMILES: Cc1c(-c2ccc(N3CCc4cccc(C(=O)Nc5nc6ccccc6s5)c4C3)nc2C(=O)NCCCCCCCCCCCC(=O)Nc2cccc3c(C4CCC(=O)NC4=O)nn(C)c23)cnn1CC12CC3CC(CC(C3)C1)C2]

### 2-(5-(1-((3r,5r,7r)-adamantan-1-ylmethyl)-5-methyl-1H-pyrazol-4-yl)-6-((12-((3-(2,6-dioxopiperidin-3-yl)-1-methyl-1H-indazol-7-yl)amino)-12-oxododecyl)carbamoyl)pyridin-2-yl)-N-(benzo[d]thiazol-2-yl)-1,2,3,4-tetrahydroisoquinoline-8-carboxamide

### Step A. Procedure for Preparation of methyl 12-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)dodecanoate

To a solution of 3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinic acid (1 g, 1.52 mmol, 1 equiv.) in DMF (10 mL) was added methyl 12-aminododecanoate; hydrochloride (403.48 mg, 1.52 mmol, 1 equiv.), DIPEA (588.53 mg, 4.55 mmol, 793.17 μL, 3 equiv.) and HATU (577.15 mg, 1.52 mmol, 1 equiv.) at 25° C. The reaction mixture was stirred at 25° C. for 1.5 hours. The reaction mixture was diluted with H₂O (10 mL) and extracted with EtOAc (30 mL×3). The combined organic layers were washed with NaHCO₃ (10 mL×3), brine (10 mL×3), dried over Na₂SO₄, filtered, and concentrated under reduced pressure to give a residue. The residue was purified by flash silica gel chromatography (Eluent of 0˜8% MeOH/DCM) to give methyl 12-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)dodecanoate (1 g, 1.1 mmol, 74.0% yield, 97.8% purity) as a yellow oil.

MS (ESI) m/z: 871.0 [M+H]⁺.

### Step B. Procedure for Preparation of 12-(3-(1-(adamantan-1-ylmethyl)-5-methyl-11H-pyrazol-4-yl)-6-(8-(benzo[d]thiazol-2-ylcarbamoyl)-3,4-dihydroisoquinolin-2(1H)-yl)picolinamido)dodecanoic acid

To a solution of methyl 12-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)dodecanoate (970 mg, 1.11 mmol, 1 equiv.) in THE (1 mL) and H₂O (0.1 mL) was added LiOH·H₂O (140.34 mg, 3.34 mmol, 3 equiv.) at 25° C. The reaction mixture was stirred at 40° C. for 20 hours. The reaction mixture was concentrated under reduced pressure to give a residue. The residue was treated with water (4 mL). The pH was adjusted to about 3 by progressively adding 1 M HCl. The mixture was filtered and concentrated under reduced pressure to give 12-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)dodecanoic acid (658 mg, 732.3 μmol, 65.6% yield, 95.2% purity) as a yellow solid.

MS (ESI) m/z: 856.9 [M+H]⁺.

¹H NMR (400 MHz, DMSO-d₆) δ=8.06 (s, 1H), 7.97 (d, J 8.0 Hz, 1H), 7.74 (d, J 8.0 Hz, 1H), 7.62 (d, J 7.2 Hz, 1H), 7.46-7.41 (m, 2H), 7.38-7.31 (m, 2H), 7.23 (s, 1H), 6.94 (d, J 8.8 Hz, 1H), 5.02 (s, 2H), 3.88 (t, J 6.0 Hz, 2H), 3.67 (s, 2H), 3.04-2.97 (m, 4H), 2.17 (t, J 7.2 Hz, 2H), 2.04 (s, 3H), 1.92 (s, 3H), 1.67-1.55 (m, 6H), 1.52 (s, 7H), 1.49-1.44 (m, 2H), 1.30-1.25 (m, 2H), 1.24-1.17 (m, 7H), 1.11 (d, J 11.6 Hz, 6H)

### Step C. Procedure for Preparation of 2-(5-(1-((3r,5r,7r)-adamantan-1-ylmethyl)-5-methyl-1H-pyrazol-4-yl)-6-((12-((3-(2,6-dioxopiperidin-3-yl)-1-methyl-1H-indazol-7-yl)amino)-12-oxododecyl)carbamoyl)pyridin-2-yl)-N-(benzo[d]thiazol-2-yl)-1,2,3,4-tetrahydroisoquinoline-8-carboxamide

A mixture of 12-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)dodecanoic acid (80.00 mg, 93.44 μmol, 1 equiv.), 3-(7-amino-1-methyl-indazol-3-yl)piperidine-2,6-dione (26.55 mg, 102.79 μmol, 1.10 equiv.), HATU (39.08 mg, 102.79 μmol, 1.10 equiv.), TEA (47.28 mg, 467.22 μmol, 65.03 μL, 5.00 equiv.) in DMF (1 mL) was degassed and purged with N₂ three times, and then the mixture was stirred at 20° C. for 12 hours under N₂ atmosphere. The reaction mixture was concentrated under reduced pressure to give a residue. The residue was purified by prep-HPLC to give 2-(5-(1-((3r,5r,7r)-adamantan-1-ylmethyl)-5-methyl-1H-pyrazol-4-yl)-6-((12-((3-(2,6-dioxopiperidin-3-yl)-1-methyl-1H-indazol-7-yl)amino)-12-oxododecyl)carbamoyl)pyridin-2-yl)-N-(benzo[d]thiazol-2-yl)-1,2,3,4-tetrahydroisoquinoline-8-carboxamide (16.3 mg, 14.3 μmol, 15.3% yield, 96.3% purity) as a yellow solid.

MS (ESI) m/z: 1096.3 [M+1]⁺.

¹H NMR (400 MHz, CD₃OD) δ=7.91 (d, J=8.0 Hz, 1H), 7.79 (d, J=8.0 Hz, 1H), 7.68-7.63 (m, 1H), 7.56 (d, J=6.8 Hz, 1H), 7.49-7.43 (m, 2H), 7.42-7.38 (m, 1H), 7.37-7.31 (m, 3H), 7.17-7.09 (m, 2H), 7.01 (d, J=8.8 Hz, 1H), 5.13 (s, 2H), 4.41-4.33 (m, 1H), 4.09 (s, 3H), 3.92 (t, J=6.0 Hz, 2H), 3.73 (s, 2H), 3.17 (t, J=7.2 Hz, 2H), 3.07 (t, J=5.6 Hz, 2H), 2.84-2.65 (m, 2H), 2.52-2.39 (m, 3H), 2.37-2.26 (m, 1H), 2.10 (s, 3H), 1.97 (s, 3H), 1.78-1.69 (m, 5H), 1.69-1.64 (m, 3H), 1.61 (d, J=1.6 Hz, 6H), 1.47-1.38 (m, 4H), 1.36-1.27 (m, 5H), 1.23-1.16 (s, 7H)

### Example 125. Preparation of Compound 111

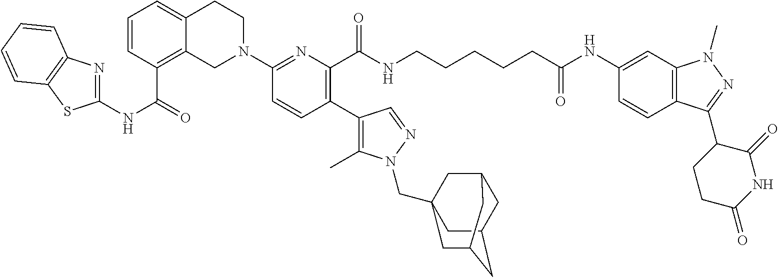 [SMILES: Cc1c(-c2ccc(N3CCc4cccc(C(=O)Nc5nc6ccccc6s5)c4C3)nc2C(=O)NCCCCCC(=O)Nc2ccc3c(C4CCC(=O)NC4=O)nn(C)c3c2)cnn1CC12CC3CC(CC(C3)C1)C2]

### 2-(5-(1-(((3r,5r,7r)-adamantan-1-yl)methyl)-5-methyl-1H-pyrazol-4-yl)-6-((6-((3-(2,6-dioxopiperidin-3-yl)-1-methyl-1H-indazol-6-yl)amino)-6-oxohexyl)carbamoyl)pyridin-2-yl)-N-(benzo[d]thiazol-2-yl)-1,2,3,4-tetrahydroisoquinoline-8-carboxamide

### Step A. Procedure for Preparation of methyl 6-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)hexanoate

To a solution of 3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinic acid (1 g, 1.52 mmol, 1 equiv.) in DMF (10 mL) and methyl 6-aminohexanoate (551.48 mg, 3.04 mmol, 2 equiv., HCl) was added HATU (692.58 mg, 1.82 mmol, 1.2 equiv.) and DIEA (588.53 mg, 4.55 mmol, 793.17 μL, 3 equiv.). The mixture was stirred at 25° C. for 1 hour. The mixture was treated dropwise with water, filtered, and concentrated under reduced pressure. The residue was purified by flash silica gel chromatography (Eluent of 0-60% ethyl acetate/petroleum ether) to give methyl 6-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)hexanoate (690 mg, 816.4 μmol, 53.7% yield, 93% purity) as a yellow solid.

MS (ESI) m/z: 786.9 [M+H]⁺

### Step B. Procedure for Preparation of 6-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)hexanoic acid

To a solution of methyl 6-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)hexanoate (670 mg, 852.42 μmol, 1 equiv.) in THE (7 mL) was added LiOH·H₂O (1 M, 2.56 mL, 3 equiv.). The mixture was stirred at 25° C. for 12 hours. The reaction mixture was added HCl (1 M) to pH=6, filtered to give a filter cake. The crude product was triturated with CH₃CN (5 mL) at 25° C. for 0.5 hour to give 6-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)hexanoic acid (552.07 mg, 642.20 μmol, 75.3% yield, 89.8% purity) as a yellow solid.

MS (ESI) m/z: 772.1 [M+H]⁺

¹H NMR (400 MHz, DMSO-d₆) δ=8.10 (t, J=5.2 Hz, 1H), 7.90 (d, J=7.6 Hz, 1H), 7.66 (t, J=6.8 Hz, 2H), 7.45 (d, J=8.8 Hz, 1H), 7.41-7.26 (m, 4H), 7.26-7.21 (m, 2H), 6.93 (d, J=8.8 Hz, 1H), 5.05 (s, 2H), 3.89 (t, J=5.6 Hz, 2H), 3.04-2.98 (m, 4H), 2.10 (t, J=7.2 Hz, 2H), 2.05 (s, 3H), 1.92 (s, 3H), 1.68-1.61 (m, 4H), 1.58-1.51 (m, 10H), 1.41-1.36 (m, 2H), 1.33-1.27 (m, 2H), 1.15-1.08 (m, 2H).

### Step C. Procedure for Preparation of 2-(5-(1-(((3r,5r,7r)-adamantan-1-yl)methyl)-5-methyl-1H-pyrazol-4-yl)-6-((6-((3-(2,6-dioxopiperidin-3-yl)-1-methyl-1H-indazol-6-yl)amino)-6-oxohexyl)carbamoyl)pyridin-2-yl)-N-(benzo[d]thiazol-2-yl)-1,2,3,4-tetrahydroisoquinoline-8-carboxamide

To a solution of 6-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)hexanoic acid (80 mg, 103.63 μmol, 1 equiv.) and 3-(6-amino-1-methyl-1H-indazol-3-yl)piperidine-2,6-dione (29.44 mg, 113.99 μmol, 1.1 equiv.) in DMF (1 mL) was added HATU (47.28 mg, 124.36 μmol, 1.2 equiv.) and DIEA (40.18 mg, 310.89 μmol, 54.15 μL, 3 equiv.). The mixture was stirred at 25° C. for 12 hours. The reaction mixture was concentrated under reduced pressure to give a residue. The residue was purified by prep-HPLC to give 2-(5-(1-(((3r,5r,7r)-adamantan-1-yl)methyl)-5-methyl-1H-pyrazol-4-yl)-6-((6-((3-(2,6-dioxopiperidin-3-yl)-1-methyl-1H-indazol-6-yl)amino)-6-oxohexyl)carbamoyl)pyridin-2-yl)-N-(benzo[d]thiazol-2-yl)-1,2,3,4-tetrahydroisoquinoline-8-carboxamide (68.6 mg, 62.5 μmol, 60.3% yield, 92.1% purity) as a yellow solid.

MS (ESI) m/z: 1012.4 [M+H]⁺

¹H NMR (400 MHz, DMSO-d₆) δ=12.92-12.80 (m, 1H), 10.88 (s, 1H), 10.03 (s, 1H), 8.12-8.05 (m, 2H), 8.02 (d, J=8.0 Hz, 1H), 7.78 (d, J=8.4 Hz, 1H), 7.63-7.57 (m, 2H), 7.47-7.42 (m, 2H), 7.42-7.38 (m, 1H), 7.37-7.29 (m, 2H), 7.24 (s, 1H), 7.07 (dd, J=1.2, 8.8 Hz, 1H), 6.94 (d, J=8.8 Hz, 1H), 4.98 (s, 2H), 4.34-4.28 (m, 1H), 3.91-3.85 (m, 5H), 3.66 (s, 2H), 3.07-2.97 (m, 4H), 2.64-2.61 (m, 1H), 2.27 (t, J=7.2 Hz, 2H), 2.22-2.13 (m, 2H), 2.04 (s, 3H), 1.89 (s, 3H), 1.64-1.58 (m, 3H), 1.56-1.46 (m, 12H), 1.38-1.32 (m, 2H), 1.22-1.14 (m, 2H).

### Example 126. Preparation of Compound 112

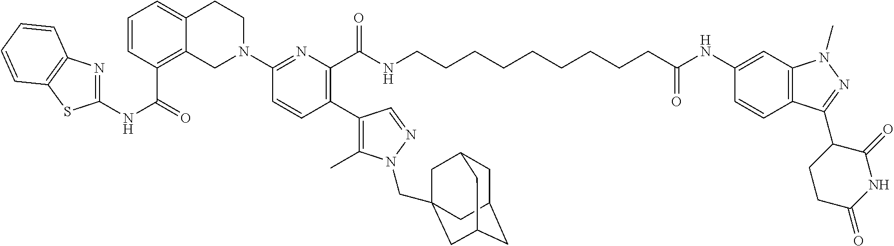 [SMILES: Cc1c(-c2ccc(N3CCc4cccc(C(=O)Nc5nc6ccccc6s5)c4C3)nc2C(=O)NCCCCCCCCCC(=O)Nc2ccc3c(C4CCC(=O)NC4=O)nn(C)c3c2)cnn1CC12CC3CC(CC(C3)C1)C2]

### 2-[5-[1-(1-adamantylmethyl)-5-methyl-pyrazol-4-yl]-6-[[10-[[3-(2,6-dioxo-3-piperidyl)-1-methyl-indazol-6-yl]amino]-10-oxo-decyl]carbamoyl]-2-pyridyl]-N-(1,3-benzothiazol-2-yl)-3,4-dihydro-1H-isoquinoline-8-carboxamide

### Step A. Procedure for Preparation of 2-[5-[1-(1-adamantylmethyl)-5-methyl-pyrazol-4-yl]-6-[[10-[[3-(2,6-dioxo-3-piperidyl)-1-methyl-indazol-6-yl]amino]-10-oxo-decyl]carbamoyl]-2-pyridyl]-N-(1,3-benzothiazol-2-yl)-3,4-dihydro-1H-isoquinoline-8-carboxamide

To a solution of 10-[[3-[1-(1-adamantylmethyl)-5-methyl-pyrazol-4-yl]-6-[8-(1,3-benzothiazol-2-ylcarbamoyl)-3,4-dihydro-1H-isoquinolin-2-yl]pyridine-2-carbonyl]amino]decanoic acid (80 mg, 96.61 μmol, 1 equiv.), HATU (36.73 mg, 96.61 μmol, 1 equiv.), and DIPEA (37.46 mg, 289.83 μmol, 50.48 μL, 3 equiv.) in DMF (1 mL) was added 3-(6-amino-1-methyl-indazol-3-yl)piperidine-2,6-dione (27.45 mg, 106.27 μmol, 1.1 equiv.). The mixture was stirred at 25° C. for 6 hours. The mixture was concentrated as a residue. The residue was purified by prep-TLC (ethyl acetate:MeOH=30:1) to give 2-[5-[1-(1-adamantylmethyl)-5-methyl-pyrazol-4-yl]-6-[[10-[[3-(2,6-dioxo-3-piperidyl)-1-methyl-indazol-6-yl]amino]-10-oxo-decyl]carbamoyl]-2-pyridyl]-N-(1,3-benzothiazol-2-yl)-3,4-dihydro-1H-isoquinoline-8-carboxamide (25.6 mg, 22.5 μmol, 23.3% yield, 93.6% purity) as a white solid.

MS (ESI) m/z: 535.1 [M+2H]⁺.

¹H NMR (400 MHz, CD₃OD) δ=8.06 (d, J=1.2 Hz, 1H), 7.90 (d, J=7.6 Hz, 1H), 7.77 (d, J=8.0 Hz, 1H), 7.61 (d, J=8.8 Hz, 1H), 7.55 (dd, J=1.2, 7.2 Hz, 1H), 7.47-7.42 (m, 2H), 7.38-7.30 (m, 4H), 7.08 (dt, J=1.6, 4.2 Hz, 1H), 7.00 (d, J=8.8 Hz, 1H), 5.14-5.10 (m, 2H), 4.34-4.30 (m, 1H), 3.97 (s, 1H), 3.94 (s, 3H), 3.93-3.90 (m, 2H), 3.71 (s, 2H), 3.66-3.53 (m, 2H), 3.16 (t, J=7.3 Hz, 2H), 3.05 (t, J=6.0 Hz, 2H), 2.80-2.69 (m, 3H), 2.46-2.29 (m, 5H), 2.23-2.16 (m, 2H), 2.09 (s, 3H), 2.05-1.94 (m, 4H), 1.70-1.65 (m, 5H), 1.61-1.56 (m, 3H), 1.32-1.26 (m, 6H), 1.20-1.18 (m, 4H).

### Example 127. Preparation of Compound 113

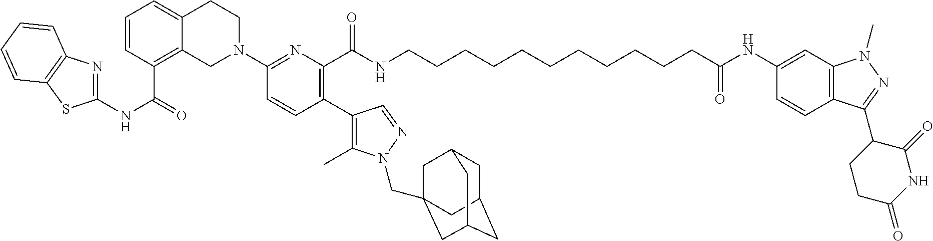 [SMILES: Cc1c(-c2ccc(N3CCc4cccc(C(=O)Nc5nc6ccccc6s5)c4C3)nc2C(=O)NCCCCCCCCCCCC(=O)Nc2ccc3c(C4CCC(=O)NC4=O)nn(C)c3c2)cnn1CC12CC3CC(CC(C3)C1)C2]

### 2-(5-(1-((3r,5r,7r)-adamantan-1-ylmethyl)-5-methyl-1H-pyrazol-4-yl)-6-((12-((3-(2,6-dioxopiperidin-3-yl)-1-methyl-1H-indazol-6-yl)amino)-12-oxododecyl)carbamoyl)pyridin-2-yl)-N-(benzo[d]thiazol-2-yl)-1,2,3,4-tetrahydroisoquinoline-8-carboxamide

### Step A. Procedure for Preparation of 2-(5-(1-((3r,5r,7r)-adamantan-1-ylmethyl)-5-methyl-1H-pyrazol-4-yl)-6-((12-((3-(2,6-dioxopiperidin-3-yl)-1-methyl-1H-indazol-6-yl)amino)-12-oxododecyl)carbamoyl)pyridin-2-yl)-N-(benzo[d]thiazol-2-yl)-1,2,3,4-tetrahydroisoquinoline-8-carboxamide

A mixture of 12-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)dodecanoic acid (60.00 mg, 70.08 μmol, 1 equiv.), 3-(6-amino-1-methyl-indazol-3-yl)piperidine-2,6-dione (18.10 mg, 70.08 μmol, 1.00 equiv.), TEA (21.27 mg, 210.24 μmol, 29.26 μL, 3.00 equiv.), HATU (29.31 mg, 77.09 μmol, 1.10 equiv.) in DMF (1 mL) was degassed and purged with N₂ and stirred at 20° C. for 3 hours under N₂ atmosphere. The reaction mixture was concentrated under reduced pressure to give a residue. The residue was purified by prep-HPLC to give 2-(5-(1-((3r,5r,7r)-adamantan-1-ylmethyl)-5-methyl-1H-pyrazol-4-yl)-6-((12-((3-(2,6-dioxopiperidin-3-yl)-1-methyl-1H-indazol-6-yl)amino)-12-oxododecyl)carbamoyl)pyridin-2-yl)-N-(benzo[d]thiazol-2-yl)-1,2,3,4-tetrahydroisoquinoline-8-carboxamide (18.6 mg, 16.1 μmol, 23.0% yield, 95.3% purity) as an off-white solid

MS (ESI) m/z: 1096.5 [M+1]⁺.

¹H NMR (400 MHz, CD₃OD) δ=8.52 (s, 1H), 8.06 (s, 1H), 7.89 (d, J=8.0 Hz, 1H), 7.77 (d, J=8.1 Hz, 1H), 7.62 (d, J=8.8 Hz, 1H), 7.56 (d, J=6.4 Hz, 1H), 7.49-7.42 (m, 2H), 7.41-7.30 (m, 4H), 7.07 (dd, J=1.6, 8.8 Hz, 1H), 7.00 (d, J=8.4 Hz, 1H), 5.12 (s, 2H), 4.32 (dd, J=5.2, 8.8 Hz, 1H), 3.95 (s, 3H), 3.91 (t, J=6.0 Hz, 2H), 3.72 (s, 2H), 3.15 (t, J=7.2 Hz, 2H), 3.06 (t, J=5.6 Hz, 2H), 2.83-2.65 (m, 2H), 2.49-2.36 (m, 3H), 2.34-2.25 (m, 1H), 2.09 (s, 3H), 1.95 (s, 3H), 1.76-1.68 (m, 5H), 1.66 (s, 3H), 1.60 (s, 6H), 1.42-1.27 (m, 9H), 1.20-1.14 (s, 7H)

### Example 128. Preparation of Compound 115

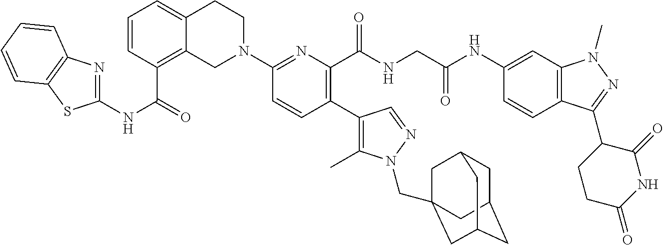 [SMILES: Cc1c(-c2ccc(N3CCc4cccc(C(=O)Nc5nc6ccccc6s5)c4C3)nc2C(=O)NCC(=O)Nc2ccc3c(C4CCC(=O)NC4=O)nn(C)c3c2)cnn1CC12CC3CC(CC(C3)C1)C2]

### 2-(5-(1-((1R,3S)-adamantan-1-ylmethyl)-5-methyl-1H-pyrazol-4-yl)-6-((2-((3-(2,6-dioxopiperidin-3-yl)-1-methyl-1H-indazol-6-yl)amino)-2-oxoethyl)carbamoyl)pyridin-2-yl)-N-(benzo[d]thiazol-2-yl)-1,2,3,4-tetrahydroisoquinoline-8-carboxamide

### Step A. Procedure for Preparation of 1-(1-adamantylmethyl)pyrazole

To a solution of 1H-pyrazole (13.50 g, 198.30 mmol, 1.00 equiv.) in DMF (200 mL) was added Cs₂CO₃ (129.22 g, 396.61 mmol, 2.00 equiv.) and 1-(bromomethyl)adamantane (45.44 g, 198.30 mmol, 1.00 equiv.). The mixture was stirred at 80° C. for 12 hours. The reaction was diluted with water (600 mL) and extracted with ethyl acetate (300 mL×2). The combined organic layers were washed with brine (150 mL×1), dried over Na₂SO₄, filtered, and concentrated under reduced pressure to give a residue. The residue was purified by column chromatography (SiO₂, petroleum ether/ethyl acetate=100/1 to 50/1) to give 1-(1-adamantylmethyl)pyrazole (42 g, 194.1 mmol, 97.9% yield) as a white solid.

MS (ESI) m/z: 217.3 [M+H]⁺.

¹H NMR (400 MHz, CDCl₃) δ=7.51 (d, J=0.8 Hz, 1H), 7.33 (d, J=2.0 Hz, 1H), 6.26 (t, J=2.0 Hz, 1H), 3.83 (s, 2H), 2.00 (s, 2H), 1.70 (s, 2H), 1.64-1.57 (m, 4H), 1.53 (s, 6H)

### Step B. Procedure for Preparation of 1-(1-adamantylmethyl)-4-iodo-pyrazole

To a solution of 1-(1-adamantylmethyl)pyrazole (38.00 g, 175.66 mmol, 1.00 equiv.) in DMF (400 mL) was added NIS (51.38 g, 228.36 mmol, 1.30 equiv.). The mixture was stirred at 40° C. for 10 hours. The reaction was diluted with water (1.5 L) and extracted with ethyl acetate (1 L×2). The combined organic layers were washed with brine (1500 mL×1), dried over Na₂SO₄, filtered, and concentrated under reduced pressure to give 1-(1-adamantylmethyl)-4-iodo-pyrazole (60.00 g, 175.3 mmol, 99.8% yield) as a red solid.

MS (ESI) m/z: 342.9 [M+H]⁺.

### Step C. Procedure for Preparation of 1-(1-adamantylmethyl)-4-iodo-5-methyl-pyrazole

To a solution of 1-(1-adamantylmethyl)-4-iodo-pyrazole (30.00 g, 87.66 mmol, 1.00 equiv.) in THE (300 mL) was added LDA (2 M, 78.90 mL, 1.80 equiv.) dropwise at −75° C. The reaction was stirred for 2 hours during which time the temperature raised to −20° C. Mel (16.18 g, 113.96 mmol, 7.09 mL, 1.30 equiv.) was added dropwise. The reaction was stirred at 25° C. for 10 hours. The reaction was diluted with water (200 mL) and extracted with ethyl acetate (200 mL×2). The combined organic layers were washed with brine (60 mL×1), dried over Na₂SO₄, filtered, and concentrated under reduced pressure to give a residue. The residue was purified by column chromatography (SiO₂, dichloromethane:methanol=100/1 to 20/1) to give a residue. The crude product was triturated with petroleum ether=100 mL at 25° C. for 30 min to give 1-(1-adamantylmethyl)-4-iodo-5-methyl-pyrazole (26 g, 72.9 mmol, 83.2% yield) as a red solid.

MS (ESI) m/z: 356.9 [M+H]⁺.

¹H NMR (400 MHz, CDCl₃) δ=7.37 (s, 1H), 3.70 (s, 2H), 2.22 (s, 3H), 1.91 (s, 3H), 1.61 (s, 2H), 1.58-1.49 (m, 4H), 1.47 (d, J=2.4 Hz, 6H)

### Step D. Procedure for Preparation of 1-(1-adamantylmethyl)-5-methyl-4-(4,4,5,5-tetramethyl-1,3,2-dioxaborolan-2-yl)pyrazole

To a solution of 1-(1-adamantylmethyl)-4-iodo-5-methyl-pyrazole (15.00 g, 42.11 mmol, 1 equiv.) in dioxane (150 mL) was added TEA (12.78 g, 126.32 mmol, 17.58 mL, 3.00 equiv.), Pd(dppf)Cl₂·CH₂Cl₂ (3.44 g, 4.21 mmol, 0.10 equiv.) and 4,4,5,5-tetramethyl-1,3,2-dioxaborolane (10.78 g, 84.21 mmol, 12.22 mL, 2.00 equiv.). The mixture was stirred at 100° C. for 2 hours. The reaction mixture was filtered and concentrated under reduced pressure to give a residue. The residue was purified by column chromatography (SiO₂, petroleum ether/ethyl acetate=100/1 to 10/1) to give 1-(1-adamantylmethyl)-5-methyl-4-(4,4,5,5-tetramethyl-1,3,2-dioxaborolan-2-yl)pyrazole (12 g, 33.6 mmol, 79.9% yield) as a white solid.

MS (ESI) m/z: 357.1 [M+H]⁺.

¹H NMR (400 MHz, CDCl₃) δ=7.71 (s, 1H), 3.74 (s, 2H), 2.44 (s, 3H), 1.99 (s, 3H), 1.73-1.68 (m, 3H), 1.64 (s, 3H), 1.60 (d, J=2.4 Hz, 6H), 1.33 (s, 12H).

### Step E. Procedure for Preparation of tert-butyl 3-(1-(adamantan-1-ylmethyl)-5-methyl-11H-pyrazol-4-yl)-6-(8-(benzo[d]thiazol-2-ylcarbamoyl)-3,4-dihydroisoquinolin-2(1H)-yl)picolinate

A mixture of 1-(1-adamantylmethyl)-5-methyl-4-(4,4,5,5-tetramethyl-1,3,2-dioxaborolan-2-yl)pyrazole (4.10 g, 11.49 mmol, 1.30 equiv.), tert-butyl 6-(8-(benzo[d]thiazol-2-ylcarbamoyl)-3,4-dihydroisoquinolin-2(1H)-yl)-3-bromopicolinate (5.00 g, 8.84 mmol, 1.00 equiv.), Ad2nBup-Pd-G3 (643.94 mg, 884.20 μmol, 0.10 equiv.), K₂CO₃ (3.67 g, 26.53 mmol, 3.00 equiv.) in dioxane (50 mL) and H₂O (15 mL) was degassed and purged with N₂ three times and then the mixture was stirred at 100° C. for 2 hours under N₂ atmosphere. The reaction was diluted with water (120 mL) and extracted with ethyl acetate (100 mL×3). The combined organic layers were washed with brine (150 mL×1), dried over Na₂SO₄, filtered and concentrated under reduced pressure to give a residue. The residue was purified by column chromatography (SiO₂, petroleum ether/ethyl acetate=100/1 to 1/1) to give tert-butyl 3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinate (6.00 g, 6.6 mmol, 74.9% yield, 79% purity) as a yellow solid.

MS (ESI) m/z: 715.1 [M+H]⁺.

### Step F. Procedure for Preparation of 3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinic acid

To a solution of tert-butyl 3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinate (12.00 g, 16.79 mmol, 1.00 equiv.) in DCM (100 mL) was added TFA (123.20 g, 1.08 mol, 80 mL, 64.37 equiv.). The mixture was stirred at 40° C. for 1 hour. The reaction was diluted with DCM (300 mL) and extracted with water (800 mL×3). The combined organic layers were washed with brine (500 mL×1), dried over Na₂SO₄, filtered, and concentrated under reduced pressure to give a residue. The crude product was triturated with MeCN (100 mL) at 25° C. for 12 hours to give 3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinic acid (11 g, 16.70 mmol, 99.47% yield) as a yellow solid.

MS (ESI) m/z: 659.1 [M+H]⁺.

¹H NMR (400 MHz, DMSO) δ=12.96-12.77 (m, 1H), 8.04 (d, J=8.0 Hz, 1H), 7.80 (d, J=8.0 Hz, 1H), 7.62 (d, J=7.6 Hz, 1H), 7.54-7.32 (m, 6H), 7.27 (s, 1H), 6.95 (d, J=8.8 Hz, 1H), 4.96 (s, 2H), 3.92-3.85 (m, 2H), 3.71 (s, 2H), 3.02 (t, J=5.2 Hz, 2H), 2.10 (s, 3H), 1.93 (s, 3H), 1.67-1.62 (m, 3H), 1.58-1.50 (m, 9H).

### Step G. Procedure for Preparation of methyl 2-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)acetate

To a solution of 3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinic acid (940.00 mg, 1.43 mmol, 1.00 equiv.) in DMF (10 mL) was added HATU (651.02 mg, 1.71 mmol, 1.20 equiv.), DIEA (553.22 mg, 4.28 mmol, 745.58 uL, 3.00 equiv.) and methyl 2-aminoacetate (214.97 mg, 1.71 mmol, 1.20 equiv., HCl). The mixture was stirred at 25° C. for 1 hour. The reaction was diluted with water (80 mL) and extracted with ethyl acetate 50 mL (50 mL×3). The combined organic layers were washed with brine 60 mL (60 mL×1), dried over Na₂SO₄, filtered, and concentrated under reduced pressure to give a residue. The residue was purified by column chromatography (SiO₂, petroleum ether/ethyl acetate=100/1 to 1/1) to give methyl 2-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)acetate (900 mg, 1.1 mmol, 82.1% yield, 95% purity) as a yellow solid.

MS (ESI) m/z: 730.1 [M+H]⁺.

### Step H. Procedure for Preparation of 2-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)acetic acid

To a solution of methyl 2-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)acetate (900.00 mg, 1.23 mmol, 1.00 equiv.) in THF (10 mL) and H₂O (3 mL) was added LiOH·H₂O (155.23 mg, 3.70 mmol, 3.00 equiv.). The mixture was stirred at 25° C. for 2 hours. The reaction mixture was acidified to pH=5 with 1M HCl and extracted with DCM (50 mL×3). The combined organic layers were washed with brine (50 mL×1), dried over Na₂SO₄, filtered, and concentrated under reduced pressure to give a residue. The crude product was triturated with petroleum ether: ethyl acetate=1:1 (10 mL) at 25° C. for 30 min to give 2-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)acetic acid (500.3 mg, 698.8 μmol, 56.6% yield) as a yellow solid.

MS (ESI) m/z: 716.2 [M+H]⁺.

¹H NMR (400 MHz, DMSO) δ=12.67-12.39 (m, 1H), 8.63-8.54 (m, 1H), 8.03 (d, J=7.8 Hz, 1H), 7.80 (d, J=8.0 Hz, 1H), 7.60 (d, J=6.8 Hz, 1H), 7.52-7.32 (m, 6H), 7.29 (s, 1H), 7.04-6.98 (m, 1H), 5.00 (s, 2H), 3.97 (t, J=5.6 Hz, 2H), 3.86 (d, J=5.6 Hz, 2H), 3.68 (s, 2H), 3.02 (t, J=5.6 Hz, 2H), 2.05 (s, 3H), 1.93 (s, 3H), 1.68-1.63 (m, 3H), 1.59-1.52 (m, 9H)

### Step I. Procedure for preparation of 2-(5-(1-((1R,3S)-adamantan-1-ylmethyl)-5-methyl-11H-pyrazol-4-yl)-6-((2-((3-(2,6-dioxopiperidin-3-yl)-1-methyl-1H-indazol-6-yl)amino)-2-oxoethyl)carbamoyl)pyridin-2-yl)-N-(benzo[d]thiazol-2-yl)-1,2,3,4-tetrahydroisoquinoline-8-carboxamide

A mixture of 2-(3-(1-(adamantan-1-ylmethyl)-5-methyl-1H-pyrazol-4-yl)-6-(8-(benzo[d]thiazol-2-ylcarbamoyl)-3,4-dihydroisoquinolin-2(1H)-yl)picolinamido)acetic acid (60 mg, 83.82 μmol, 1 equiv.), 3-(6-amino-1-methyl-indazol-3-yl)piperidine-2,6-dione (21.65 mg, 83.82 μmol, 1 equiv.), HATU (31.87 mg, 83.82 μmol, 1 equiv.), and DIPEA (32.50 mg, 251.45 μmol, 43.80 μL, 3 equiv.) in DMF (0.6 mL) was stirred at 25° C. and 1 hour, then add 2-[[3-[1-(1-adamantylmethyl)-5-methyl-pyrazol-4-yl]-6-[8-(1,3-benzothiazol-2-ylcarbamoyl)-3,4-dihydro-1H-isoquinolin-2-yl]pyridine-2-carbonyl]amino]acetic acid (60 mg, 83.82 μmol, 1 equiv.) into the mixture at 25° C. and then stirred at 25° C. for 14 hours. The mixture was filtered, and the filtrate was purified by prep-HPLC to give 2-(5-(1-((1R,3S)-adamantan-1-ylmethyl)-5-methyl-1H-pyrazol-4-yl)-6-((2-((3-(2,6-dioxopiperidin-3-yl)-1-methyl-1H-indazol-6-yl)amino)-2-oxoethyl)carbamoyl)pyridin-2-yl)-N-(benzo[d]thiazol-2-yl)-1,2,3,4-tetrahydroisoquinoline-8-carboxamide (13.9 mg, 12.8 μmol, 15.3% yield, 97.5% purity) as a yellow solid.

MS (ESI) m/z: 965.5 [M+H]⁺.

¹H NMR (400 MHz, CD₃OD) δ=8.28 (s, 1H), 7.95 (s, 1H), 7.53-7.62 (m, 4H), 7.36-7.42 (m, 3H), 7.26 (t, J=7.6 Hz, 1H), 7.15 (d, J=8.8 Hz, 1H), 7.08 (t, J=7.6 Hz, 1H), 6.98 (d, J=7.6 Hz, 1H), 5.24 (s, 2H), 4.35 (dd, J=9.2, 5.2 Hz, 1H), 4.21 (s, 2H), 4.00 (t, J=6.0 Hz, 2H), 3.89 (s, 3H), 3.72 (s, 2H), 3.11 (t, J=6.0 Hz, 2H), 2.74-2.84 (m, 2H), 2.44-2.52 (m, 1H), 2.34 (dd, J=13.2, 6.0 Hz, 1H), 2.11 (s, 2H), 1.92 (s, 3H), 1.68-1.71 (m, 2H), 1.57-1.63 (m, 10H).

### Example 129. Preparation of Compound 118

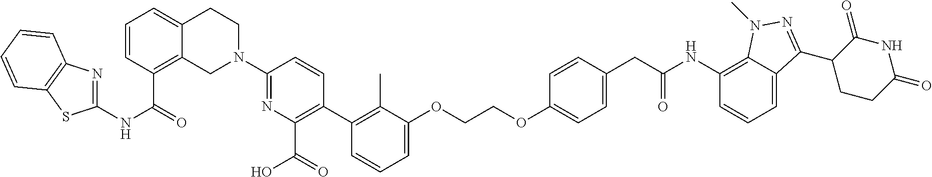 [SMILES: Cc1c(OCCOc2ccc(CC(=O)Nc3cccc4c(C5CCC(=O)NC5=O)nn(C)c34)cc2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[2-[4-[2-[[3-(2,6-dioxo-3-piperidyl)-1-methyl-indazol-7-yl]amino]-2-oxo-ethyl]phenoxy]ethoxy]-2-methyl-phenyl]pyridine-2-carboxylic acid

### Step A. Procedure for Preparation of 1-bromo-3-(2-bromoethoxy)-2-methylbenzene

A mixture of 3-bromo-2-methylphenol (2 g, 10.69 mmol, 1 equiv.), 1,2-dibromoethane (10.00 g, 53.23 mmol, 4.02 mL, 5.00 equiv.) and K₂CO₃ (4.41 g, 31.94 mmol, 3 equiv.) in CH₃CN (20 mL) was degassed and purged with N₂ and stirred at 80° C. for 12 hours. The reaction mixture was concentrated and diluted with water 30 mL and extracted with EtOAc (30 mL×3). The combined organic layers were washed with brine (30 mL×3), dried over anhydrous sodium sulfate, filtered, and concentrated under reduced pressure to give a residue. The residue was purified by flash silica gel chromatography (Eluent of 0˜5% ethyl acetate/petroleum ether) to give 1-bromo-3-(2-bromoethoxy)-2-methylbenzene (1.8 g, 6.1 mmol, 57.5% yield) as a yellow oil.

¹H NMR (400 MHz, CD₃OD) δ=7.19-7.15 (m, 1H), 7.04 (t, J=8.0 Hz, 1H), 6.88 (d, J=8.0 Hz, 1H), 4.33-4.28 (m, 2H), 3.73 (t, J=5.6 Hz, 2H), 2.34 (s, 3H)

### Step B. Procedure for Preparation of methyl 2-(4-(2-(3-bromo-2-methylphenoxy)ethoxy)phenyl)acetate

A mixture of 1-bromo-3-(2-bromoethoxy)-2-methylbenzene (200 mg, 680.31 μmol, 1 equiv.), methyl 2-(4-hydroxyphenyl)acetate (135.66 mg, 816.37 μmol, 1.2 equiv.), and K₂CO₃ (282.07 mg, 2.04 mmol, 3 equiv.) in CH₃CN (3 mL) was degassed and purged with N₂ three times, and then the mixture was stirred at 60° C. for 16 hours under N₂ atmosphere. The reaction mixture was filtered and concentrated under reduced pressure to give a residue. The residue was purified by prep-TLC (SiO₂, petroleum ether:ethyl acetate=8:1) to give methyl 2-(4-(2-(3-bromo-2-methylphenoxy)ethoxy)phenyl)acetate (150 mg, 395.5 μmol, 58.1% yield) as a colorless oil.

¹H NMR (400 MHz, CD₃OD) δ=7.23-7.16 (m, 3H), 7.07 (t, J=8.0 Hz, 1H), 6.99-6.93 (m, 3H), 4.36-4.33 (m, 4H), 3.69 (s, 3H), 3.60 (s, 2H), 2.29 (s, 3H)

### Step C. Procedure for Preparation of tert-butyl 6-(8-(benzo[d]thiazol-2-ylcarbamoyl)-3,4-dihydroisoquinolin-2(1H)-yl)-3-(3-(2-(4-(2-methoxy-2-oxoethyl)phenoxy)ethoxy)-2-methylphenyl)picolinate

Tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (218.05 mg, 355.97 μmol, 0.9 equiv.), methyl 2-(4-(2-(3-bromo-2-methylphenoxy)ethoxy)phenyl)acetate (150 mg, 395.52 μmol, 1 equiv.), KF (1.5 M, 791.05 μL, and Ad₂nBup-Pd-G₃ (28.80 mg, 39.55 μmol, 0.1 equiv.) were taken up into a microwave tube in 1,4-dioxane (4 mL). The sealed tube was heated at 100° C. for 2 hours under microwave. The reaction mixture was diluted with water (30 mL) and extracted with EtOAc (30 mL×3). The combined organic layers were washed with brine (30 mL×2), dried over anhydrous sodium sulfate, filtered, and concentrated under reduced pressure to give a residue. The residue was purified by prep-TLC (SiO₂, petroleum ether:ethyl acetate=1.5:1) to give tert-butyl 6-(8-(benzo[d]thiazol-2-ylcarbamoyl)-3,4-dihydroisoquinolin-2(1H)-yl)-3-(3-(2-(4-(2-methoxy-2-oxoethyl)phenoxy)ethoxy)-2-methylphenyl)picolinate (220 mg, 280.2 μmol, 70.8% yield) as a yellow oil.

MS (ESI) m/z: 785.4 [M+H]⁺

### Step D. Procedure for Preparation of 2-(4-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethoxy)phenyl)acetic acid

A mixture of tert-butyl 6-(8-(benzo[d]thiazol-2-ylcarbamoyl)-3,4-dihydroisoquinolin-2(1H)-yl)-3-(3-(2-(4-(2-methoxy-2-oxoethyl)phenoxy)ethoxy)-2-methylphenyl)picolinate (220 mg, 280.28 μmol, 1 equiv.) and LiOH·H₂O (35.29 mg, 840.85 μmol, 3 equiv.) in THE (1.6 mL) and H₂O (0.4 mL) was degassed and purged with N₂ and stirred at 25° C. for 1 hour under N₂ atmosphere. The reaction mixture was diluted with water (30 mL) and extracted with EtOAc (30 mL×3). The combined organic layers were washed with brine (30 mL×3), dried over anhydrous sodium sulfate, filtered and concentrated under reduced pressure to give 2-(4-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethoxy)phenyl)acetic acid (380 mg, crude) as a yellow solid.

MS (ESI) m/z: 771.6 [M+H]⁺

### Step E. Procedure for Preparation of tert-butyl 6-[8-(1,3-benzothiazol-2-ylcarbamoyl)-3,4-dihydro-1H-isoquinolin-2-yl]-3-[3-[2-[4-[2-[[3-(2,6-dioxo-3-piperidyl)-1-methyl-indazol-7-yl]amino]-2-oxo-ethyl]phenoxy]ethoxy]-2-methyl-phenyl]pyridine-2-carboxylate

A mixture of 2-[4-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]ethoxy]phenyl]acetic acid (180 mg, 233.50 μmol, 1 equiv.), 3-(7-amino-1-methyl-indazol-3-yl)piperidine-2,6-dione (60.31 mg, 233.50 μmol, 1 equiv.), DIEA (90.53 mg, 700.50 μmol, 122.01 μL, 3 equiv.) in DMF (3 mL) was degassed and purged with N₂ and stirred at 25° C. for 5 minutes. After 5 minutes, added HATU (106.54 mg, 280.20 μmol, 1.2 equiv.), the mixture was stirred at 40° C. for 1 hour under N₂ atmosphere. The reaction mixture was diluted with water (20 mL) and extracted with EtOAc (25 mL×3). The combined organic layers were washed with brine (15 mL×3), dried over anhydrous sodium sulfate, filtered and concentrated under reduced pressure to give a residue. The residue was used in the next step without purification. The compound tert-butyl 6-[8-(1,3-benzothiazol-2-ylcarbamoyl)-3,4-dihydro-1H-isoquinolin-2-yl]-3-[3-[2-[4-[2-[[3-(2,6-dioxo-3-piperidyl)-1-methyl-indazol-7-yl]amino]-2-oxo-ethyl]phenoxy]ethoxy]-2-methyl-phenyl]pyridine-2-carboxylate (200 mg, crude) was obtained as a yellow oil.

MS (ESI) m/z: 1011.3 [M+H]⁺.

### Step F. Procedure for Preparation of 6-[8-(1,3-benzothiazol-2-ylcarbamoyl)-3,4-dihydro-1H-isoquinolin-2-yl]-3-[3-[2-[4-[2-[[3-(2,6-dioxo-3-piperidyl)-1-methyl-indazol-7-yl]amino]-2-oxo-ethyl]phenoxy]ethoxy]-2-methyl-phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[3-[2-[4-[2-[[3-(2,6-dioxo-3-piperidyl)-1-methyl-indazol-7-yl]amino]-2-oxo-ethyl]phenoxy]ethoxy]-2-methyl-phenyl]pyridine-2-carboxylate (200 mg, 197.79 μmol, 1 equiv.) in DCM (1 mL) and TFA (1 mL) was degassed and purged with N₂ and stirred at 40° C. for 3 hours under N₂ atmosphere. The reaction mixture was filtered and concentrated under reduced pressure to give a residue. The residue was purified by prep-HPLC. The compound 6-[8-(1,3-benzothiazol-2-ylcarbamoyl)-3,4-dihydro-1H-isoquinolin-2-yl]-3-[3-[2-[4-[2-[[3-(2,6-dioxo-3-piperidyl)-1-methyl-indazol-7-yl]amino]-2-oxo-ethyl]phenoxy]ethoxy]-2-methyl-phenyl]pyridine-2-carboxylic acid (25.65 mg, 26.6 μmol, 13.4% yield, 99.2% purity) was obtained as a yellow solid.

MS (ESI) m/z: 956.2 [M+H]⁺

¹H NMR (400 MHz, DMSO-d₆) δ=12.88-12.80 (m, 1H), 10.87 (s, 1H), 9.99 (s, 1H), 8.03 (d, J=7.6 Hz, 1H), 7.78 (d, J=8.4 Hz, 1H), 7.64-7.55 (m, 2H), 7.49-7.42 (m, 3H), 7.42-7.24 (m, 5H), 7.14-7.04 (m, 3H), 7.00-6.94 (m, 4H), 6.67 (d, J=7.6 Hz, 1H), 4.98 (s, 2H), 4.37-4.30 (m, 5H), 3.91 (t, J=5.6 Hz, 2H), 3.87 (s, 3H), 3.65 (s, 2H), 3.02 (s, 2H), 2.70-2.64 (m, 2H), 2.19-2.09 (m, 2H), 1.89 (s, 3H)

### Example 130. Preparation of Compound 123

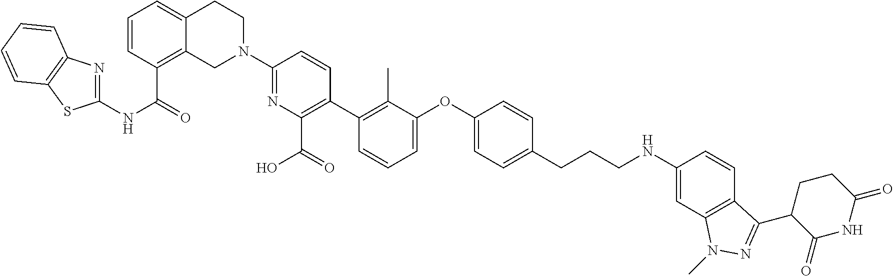 [SMILES: Cc1c(Oc2ccc(CCCNc3ccc4c(C5CCC(=O)NC5=O)nn(C)c4c3)cc2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[4-[3-[[3-(2,6-dioxo-3-piperidyl)-1-methyl-indazol-6-yl]amino]propyl]phenoxy]-2-methyl-phenyl]pyridine-2-carboxylic acid

### Step A. Procedure for Preparation of ethyl 3-[4-(3-bromo-2-methyl-phenoxy)phenyl]propanoate

A mixture of ethyl 3-(4-hydroxyphenyl)propanoate (5 g, 25.74 mmol, 1 equiv.), 1,3-dibromo-2-methyl-benzene (12.87 g, 51.49 mmol, 2 equiv.), Cs₂CO₃ (10.07 g, 30.89 mmol, 1.2 equiv.) and 2,2,6,6-tetramethylheptane-3,5-dione (1.19 g, 6.44 mmol, 1.33 mL, 0.25 equiv.) in NMP (50 mL) was degassed and purged with N₂ and then CuI (2.45 g, 12.87 mmol, 0.5 equiv.) was added to the mixture and the mixture was stirred at 120° C. for 12 hours under N₂ atmosphere. The reaction mixture was filtered, and the filtrate was diluted with water 30 mL and extracted with ethyl acetate (40 mL×2). The combined organic layers were washed with brine (20 mL×2), dried over Na₂SO₄, filtered and concentrated under reduced pressure to give a residue. The residue was purified by flash silica gel chromatography (Eluent of 0˜₃₀% ethyl acetate/petroleum ether). The compound ethyl 3-[4-(3-bromo-2-methyl-phenoxy)phenyl]propanoate (7.6 g, 20.9 mmol, 81.2% yield) was obtained as a colorless oil.

¹H NMR (400 MHz, CDCl₃) δ=7.35 (d, J=8.0 Hz, 1H), 7.15 (d, J=8.4 Hz, 2H), 7.01 (t, J=8.0 Hz, 1H), 6.86-6.80 (m, 3H), 4.14 (q, J=7.2 Hz, 2H), 2.93 (t, J=8.0 Hz, 2H), 2.61 (t, J 8.0 Hz, 2H), 2.34 (s, 3H), 1.25 (t, J=7.2 Hz, 3H)

### Step B. Procedure for Preparation of 3-[4-(3-bromo-2-methyl-phenoxy)phenyl]propan-1-ol

A mixture of ethyl 3-[4-(3-bromo-2-methyl-phenoxy)phenyl]propanoate (7.6 g, 20.92 mmol, 1 equiv.) in THE (70 mL) was slowly added LiAlH₄ (794.01 mg, 20.92 mmol, 1 equiv.) at 0° C., then the mixture was stirred at 25° C. for 2 hours under N₂ atmosphere The mixture was poured into ice-water (150 mL) slowly and the pH was acidized to pH 4-5, extracted with EtOAc (60 mL×3), washed with brine (30 mL×2), dried by sodium sulfate, filtered and concentrated under reduced pressure. The compound 3-[4-(3-bromo-2-methyl-phenoxy)phenyl]propan-1-ol (6 g, 18.6 mmol, 89.2% yield) was obtained as a yellow oil.

¹H NMR (HNMR, 400 MHz, CDCl₃) δ=7.32 (d, J=8.0 Hz, 1H), 7.13 (d, J=8.4 Hz, 2H), 6.98 (t, J=8.0 Hz, 1H), 6.85-6.79 (m, 3H), 3.67 (t, J=6.4 Hz, 2H), 2.70-2.66 (m, 2H), 2.33 (s, 3H), 1.91-1.83 (m, 2H)

### Step C. Procedure for Preparation of 3-[4-(3-bromo-2-methyl-phenoxy)phenyl]propanal

To a solution of oxalyl dichloride (2.37 g, 18.68 mmol, 1.64 mL, 2 equiv.) in DCM (5 mL) was added the mixture of DMSO (2.92 g, 37.36 mmol, 2.92 mL, 4 equiv.) in DCM (5 mL) under −70° C. and stirred for 0.5 hours. And 3-[4-(3-bromo-2-methyl-phenoxy)phenyl]propan-1-ol (3 g, 9.34 mmol, 1 equiv.) in DCM (5 mL) was stirred for 10 mins and added into the mixture for 20 mins. TEA (5.67 g, 56.04 mmol, 7.80 mL, 6 equiv.) was added into the mixture and stirred at −70° C. for 2 hours. The reaction mixture was quenched by addition of water 60 mL at 0° C., and then extracted with DCM 90 mL (30 mL×2). The combined organic layers were washed with brine 40 mL (20 mL×2), dried over Na₂SO₄, filtered, and concentrated under reduced pressure to give a residue. The residue was purified by flash silica gel chromatography (Eluent of 0˜15% ethyl acetate/petroleum ether). The compound 3-[4-(3-bromo-2-methyl-phenoxy)phenyl]propanal (2.6 g, 8.1 mmol, 87.21% yield) was obtained as a yellow oil.

¹H NMR (HNMR, 400 MHz, CDCl₃) δ=9.84 (s, 1H), 7.35 (d, J=8.0 Hz, 1H), 7.15 (d, J=8.4 Hz, 2H), 7.02 (t, J=8.0 Hz, 1H), 6.86-6.81 (m, 3H), 2.97-2.91 (m, 2H), 2.82-2.76 (m, 2H), 2.34 (s, 3H)

### Step D. Procedure for Preparation of tert-butyl 6-[8-(1,3-benzothiazol-2-ylcarbamoyl)-3,4-dihydro-1H-isoquinolin-2-yl]-3-[2-methyl-3-[4-(3-oxopropyl)phenoxy]phenyl] pyridine-2-carboxylate

A mixture of 3-[4-(3-bromo-2-methyl-phenoxy)phenyl]propanal (100 mg, 313.29 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (153.52 mg, 250.63 μmol, 0.8 equiv.), Ad₂nBuP Pd G₃(cataCXium® A Pd G₃) (45.63 mg, 62.66 μmol, 0.2 equiv.), K₂CO₃ (1.5 M, 313.29 μL, 1.5 equiv.) in 1,4-dioxane (1 mL) was degassed and purged with N₂ and stirred at 80° C. for 2 hours under N₂ atmosphere. The reaction mixture was concentrated under reduced pressure to give a residue. The residue was purified by prep-TLC (SiO₂, DCM:MeOH=10:1). The compound tert-butyl 6-[8-(1,3-benzothiazol-2-ylcarbamoyl)-3,4-dihydro-1H-isoquinolin-2-yl]-3-[2-methyl-3-[4-(3-oxopropyl)phenoxy]phenyl] pyridine-2-carboxylate (100 mg, 107.3 μmol, 11.4% yield, 77.7% purity) was obtained as a yellow oil.

MS (ESI) m/z: 725.2 [M+H]⁺

### Step E. Procedure for Preparation of tert-butyl 6-[8-(1,3-benzothiazol-2-ylcarbamoyl)-3,4-dihydro-1H-isoquinolin-2-yl]-3-[3-[4-[3-[[3-(2,6-dioxo-3-piperidyl)-1-methyl-indazol-6-yl]amino]propyl]phenoxy]-2-methyl-phenyl]pyridine-2-carboxylate

A mixture of tert-butyl 6-[8-(1,3-benzothiazol-2-ylcarbamoyl)-3,4-dihydro-1H-isoquinolin-2-yl]-3-[2-methyl-3-[4-(3-oxopropyl)phenoxy]phenyl] pyridine-2-carboxylate (100 mg, 137.96 μmol, 1 equiv.), 3-(6-amino-1-methyl-indazol-3-yl)piperidine-2,6-dione (35.63 mg, 137.96 μmol, 1 equiv.), HCOOH (6.63 mg, 137.96 μmol, 1 equiv.), and NaBH(OAc)₃ (43.86 mg, 206.94 μmol, 1.5 equiv.) in DCM (1 mL) was stirred at 25° C. for 15 hours under N₂ atmosphere. The reaction mixture was concentrated under reduced pressure to give a residue. The crude product tert-butyl 6-[8-(1,3-benzothiazol-2-ylcarbamoyl)-3,4-dihydro-1H-isoquinolin-2-yl]-3-[3-[4-[3-[[3-(2,6-dioxo-3-piperidyl)-1-methyl-indazol-6-yl]amino]propyl]phenoxy]-2-methyl-phenyl]pyridine-2-carboxylate (120 mg, crude) was obtained as yellow solid and used into the next step without further purification.

MS (ESI) m/z: 969.4 [M+H]⁺.

### Step F. Procedure for Preparation of 6-[8-(1,3-benzothiazol-2-ylcarbamoyl)-3,4-dihydro-1H-isoquinolin-2-yl]-3-[3-[4-[3-[[3-(2,6-dioxo-3-piperidyl)-1-methyl-indazol-6-yl]amino]propyl]phenoxy]-2-methyl-phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[3-[4-[3-[[3-(2,6-dioxo-3-piperidyl)-1-methyl-indazol-6-yl]amino]propyl]phenoxy]-2-methyl-phenyl]pyridine-2-carboxylate (120 mg, 124.08 μmol, 1 equiv.), TFA (42.44 mg, 372.23 μmol, 27.56 μL, 3 equiv.) in DCM (1 mL) was stirred at 25° C. for 44 hours under N₂ atmosphere. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[4-[3-[[3-(2,6-dioxo-3-piperidyl)-1-methyl-indazol-6-yl]amino]propyl]phenoxy]-2-methyl-phenyl]pyridine-2-carboxylic acid (29.69 mg, 22.8 μmol, 18.4% yield, 69.9% purity) as a white solid.

MS (ESI) m/z: 911.4 [M+H]⁺

¹H NMR (400 MHz, DMSO-d₆) δ=10.86 (s, 1H), 8.08 (d, J=8.0 Hz, 1H), 7.84 (d, J=8.0 Hz, 1H), 7.68 (d, J=7.6 Hz, 1H), 7.57 (d, J=8.8 Hz, 1H), 7.55-7.48 (m, 2H), 7.45-7.37 (m, 3H), 7.34-7.15 (m, 4H), 7.04 (d, J=8.4 Hz, 1H), 6.94 (d, J=7.6 Hz, 1H), 6.89 (d, J=8.0 Hz, 3H), 6.59 (d, J=8.4 Hz, 1H), 6.32 (s, 1H), 6.00 (s, 1H), 5.05 (s, 2H), 4.23 (dd, J=5.2, 8.4 Hz, 1H), 3.98 (t, J=5.6 Hz, 2H), 3.84 (s, 3H), 3.17-3.12 (m, 2H), 3.09 (t, J=5.2 Hz, 2H), 2.76-2.72 (m, 2H), 2.66 (t, J=6.4 Hz, 2H), 2.36-2.25 (m, 2H), 2.24-2.10 (m, 2H), 1.96 (s, 3H)

### Example 131. Preparation of Compound 124a

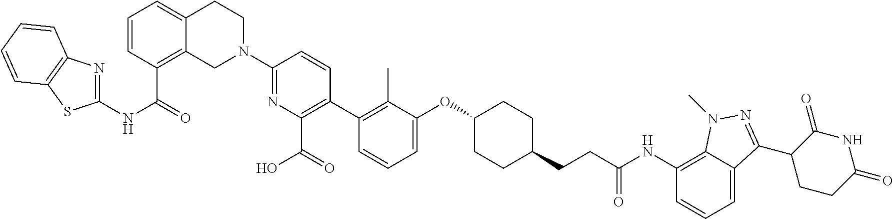 [SMILES: Cc1c(O[C@H]2CC[C@H](CCC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-(3-((3-(2,6-dioxopiperidin-3-yl)-1-methyl-1H-indazol-7-yl)amino)-3-oxopropyl)cyclohexyl)oxy)-2-methylphenyl)picolinic acid

### Step A. Procedure for Preparation of ethyl 4-(3-bromo-2-methyl-phenoxy)cyclohexanecarboxylate

To a solution of ethyl 4-hydroxycyclohexanecarboxylate (10 g, 58.06 mmol, 1 equiv.), 3-bromo-2-methyl-phenol (11.95 g, 63.87 mmol, 1.1 equiv.), and PPh₃ (24.37 g, 92.90 mmol, 1.6 equiv.) in THE (100 mL) was added DIAD (18.79 g, 92.90 mmol, 18.06 mL, 1.6 equiv.) under 0° C. Then the mixture was stirred at 25° C. for 16 hours. The reaction mixture was concentrated under reduced pressure to give a residue. The residue was purified by flash silica gel chromatography (Eluent of 0˜10% ethyl acetate/petroleum ether) to give ethyl 4-(3-bromo-2-methyl-phenoxy)cyclohexanecarboxylate (3.6 g, 10.55 mmol, 18.17% yield) as a white solid.

¹H NMR (400 MHz, CDCl₃) δ=7.14 (d, J=8.0 Hz, 1H), 6.97 (t, J=8.0 Hz, 1H), 6.80 (d, J=8.0 Hz, 1H), 4.20-4.11 (m, 3H), 2.39-2.35 (m, 2H), 2.30 (s, 3H), 2.19-2.14 (m, 2H), 2.09-2.04 (m, 2H), 1.62-1.52 (m, 5H), 1.26 (t, J=6.8 Hz, 3H).

### Step B. Procedure for Preparation of [4-(3-bromo-2-methyl-phenoxy)cyclohexyl]methanol

To a solution of ethyl 4-(3-bromo-2-methyl-phenoxy)cyclohexanecarboxylate (3.6 g, 10.55 mmol, 1 equiv.) in THE (15 mL) was added LiAlH₄ (480.49 mg, 12.66 mmol, 1.2 equiv.) under 0° C. Then the mixture was stirred at 25° C. for 1.5 hours. The reaction mixture was quenched by addition Na₂SO₄·10H₂O (1 g), and then extracted with ethyl acetate (10 mL×3). The combined organic layers were dried over Na₂SO₄, filtered, and concentrated under reduced pressure to give compound [4-(3-bromo-2-methyl-phenoxy)cyclohexyl]methanol (2.96 g, crude) as a white solid.

¹H NMR (400 MHz, CDCl₃) δ=7.13 (d, J=8.0 Hz, 1H), 6.97 (t, J=8.0 Hz, 1H), 6.81 (d, J=8.4 Hz, 1H), 4.13-4.08 (m, 1H), 3.50 (d, J=6.4 Hz, 2H), 2.30 (s, 3H), 2.20-2.14 (m, 2H), 1.95-1.86 (m, 2H), 1.53-1.45 (m, 3H), 1.14-1.04 (m, 2H).

### Step C. Procedure for Preparation of 4-(3-bromo-2-methyl-phenoxy)cyclohexanecarbaldehyde

To a solution of DMSO (3.09 g, 39.57 mmol, 3.09 mL, 4 equiv.) in DCM (10 mL) was added dropwise a solution of (COCl)₂ (2.51 g, 19.79 mmol, 1.73 mL, 2 equiv.) in DCM (2 mL) at −70° C. under N₂ atmosphere. The mixture was stirred at −70° C. for 1 hour. After that, [4-(3-bromo-2-methyl-phenoxy)cyclohexyl]methanol (2.96 g, 9.89 mmol, 1 equiv.) in DCM (10 mL) was added dropwise at −70° C. The solution was stirred for 1 hour at −70° C. Then TEA (6.01 g, 59.36 mmol, 8.26 mL, 6 equiv.) was added into the solution. The solution was stirred at −70° C. for 0.5 hour under N₂ atmosphere. The reaction mixture was quenched by addition H₂O (10 mL), and then diluted with DCM (10 mL) and extracted with DCM (10 mL×3). The combined organic layers were washed with H₂O 5 mL×3, dried over anhydrous sodium sulfate, filtered and concentrated under reduced pressure to give compound 4-(3-bromo-2-methyl-phenoxy)cyclohexanecarbaldehyde (3 g, crude) as a white solid.

¹H NMR (400 MHz, DMSO-d₆) δ=9.62-9.59 (m, 1H), 7.16-7.01 (m, 3H), 4.36-4.23 (m, 1H), 2.39-2.31 (m, 1H), 2.25-2.18 (m, 3H), 2.03-1.92 (m, 4H), 1.50-1.38 (m, 4H).

### Step D. Procedure for Preparation of ethyl (E)-3-[4-(3-bromo-2-methyl-phenoxy)cyclohexyl]prop-2-enoate

To a solution of NaH (56.52 mg, 1.41 mmol, 60% purity, 2.1 equiv.) in THE (3 mL) was added ethyl 2-diethoxyphosphorylacetate (301.75 mg, 1.35 mmol, 267.04 μL, 2 equiv.) under 0° C. Then 4-(3-bromo-2-methyl-phenoxy)cyclohexanecarbaldehyde (200 mg, 672.98 μmol, 1 equiv.) was added and the mixture was stirred at 25° C. for 6 hours. The reaction mixture was quenched by addition sat. NH₄Cl (5 mL) at 0° C., and then diluted with ethyl acetate (5 mL) and extracted with ethyl acetate (10 mL×3). The combined organic layers were washed with H₂O (10 mL), dried over Na₂SO₄, filtered, and concentrated under reduced pressure to give a residue. The residue was purified by flash silica gel chromatography (Eluent of 0-10% ethyl acetate/petroleum ether) to give ethyl (E)-3-[4-(3-bromo-2-methyl-phenoxy)cyclohexyl]prop-2-enoate (140 mg, 362.1 μmol, 53.8% yield, 95% purity) as a colorless oil.

¹H NMR (400 MHz, CDCl₃) δ=7.14 (d, J=8.0 Hz, 1H), 7.01-6.87 (m, 2H), 6.80 (d, J=8.4 Hz, 1H), 5.82 (dd, J=1.2, 15.6 Hz, 1H), 4.21-4.15 (m, 2H), 4.16-4.07 (m, 1H), 2.30 (s, 3H), 2.26-2.16 (m, 3H), 1.97-1.89 (m, 2H), 1.58-1.48 (m, 2H), 1.33-1.26 (m, 5H).

### Step E. Procedure for Preparation of ethyl 3-[4-(3-bromo-2-methyl-phenoxy)cyclohexyl]propanoate

A mixture of ethyl (E)-3-[4-(3-bromo-2-methyl-phenoxy)cyclohexyl]prop-2-enoate (1.6 g, 4.36 mmol, 1 equiv.) and PtO₂ (98.92 mg, 435.64 μmol, 0.1 equiv.) in EtOH (15 mL) was degassed and purged with H₂ three times, and then the mixture was stirred at 25° C. for 3 hours under H₂ (15 Psi) atmosphere. The reaction mixture was filtered and concentrated under reduced pressure to give a residue. The residue was purified by prep-HPLC to give ethyl 3-[4-(3-bromo-2-methyl-phenoxy)cyclohexyl]propanoate (1.4 g, 3.7 mmol, 87.0% yield) as a white solid.

¹H NMR (400 MHz, DMSO-d₆) δ=7.17-7.10 (m, 1H), 7.06 (t, J=8.0 Hz, 1H), 7.03-6.99 (m, 1H), 4.27-4.17 (m, 1H), 4.11-4.04 (m, 2H), 2.33-2.26 (m, 2H), 2.20 (s, 3H), 2.03 (d, J=10.0 Hz, 2H), 1.75 (d, J=12.0 Hz, 2H), 1.55-1.42 (m, 2H), 1.39-1.21 (m, 3H), 1.17 (t, J=7.2 Hz, 3H), 1.11-0.97 (m, 2H).

### Step F. Procedure for Preparation of tert-butyl 6-(8-(benzo[d]thiazol-2-ylcarbamoyl)-3,4-dihydroisoquinolin-2(1H)-yl)-3-(3-(((1r,4r)-4-(3-ethoxy-3-oxopropyl)cyclohexyl)oxy)-2-methylphenyl)picolinate

A mixture of ethyl 3-[4-(3-bromo-2-methyl-phenoxy)cyclohexyl]propanoate (200 mg, 541.58 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (546.40 mg, 758.21 μmol, 85% purity, 1.4 equiv.), [2-(2-aminophenyl)phenyl]palladium(1+); bis(1-adamantyl)-butyl-phosphane; methanesulfonate (78.88 mg, 108.32 μmol, 0.2 equiv.), and K₂CO₃ (1.5 M, 541.58 μL, 1.5 equiv.) in dioxane (2.5 mL) was degassed and purged with N₂ and stirred at 100° C. for 1 hour under microwave. The reaction mixture was filtered and concentrated under reduced pressure to give a residue. The mixture was purified by flash silica gel chromatography (Eluent of 0˜50% ethyl acetate/petroleum ether) to give tert-butyl 6-(8-(benzo[d]thiazol-2-ylcarbamoyl)-3,4-dihydroisoquinolin-2(1H)-yl)-3-(3-(((1r,4r)-4-(3-ethoxy-3-oxopropyl)cyclohexyl)oxy)-2-methylphenyl)picolinate (440 mg, crude) as a white solid.

¹H NMR (400 MHz, DMSO-d₆) δ=8.02 (d, J=7.6 Hz, 1H), 7.78 (d, J=8.0 Hz, 1H), 7.60 (d, J=7.5 Hz, 1H), 7.50-7.40 (m, 3H), 7.40-7.30 (m, 2H), 7.12-7.03 (m, 1H), 6.99-6.89 (m, 2H), 6.59-6.49 (m, 1H), 5.03-4.90 (m, 2H), 4.27-4.14 (m, 1H), 4.03 (d, J=6.8 Hz, 2H), 3.87 (t, J=5.6 Hz, 2H), 3.29 (s, 2H), 3.03 (t, J=5.6 Hz, 2H), 2.29 (t, J=7.6 Hz, 2H), 2.05 (d, J=11.2 Hz, 1H), 1.84 (s, 3H), 1.79-1.70 (m, 2H), 1.50-1.42 (m, 2H), 1.23 (d, J=8.4 Hz, 2H), 1.19-1.17 (m, 3H), 1.07 (s, 2H), 1.00 (s, 9H).

### Step G. Procedure for Preparation of 3-((1r,4r)-4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)cyclohexyl)propanoic acid

A mixture of tert-butyl 6-(8-(benzo[d]thiazol-2-ylcarbamoyl)-3,4-dihydroisoquinolin-2(1H)-yl)-3-(3-(((1r,4r)-4-(3-ethoxy-3-oxopropyl)cyclohexyl)oxy)-2-methylphenyl)picolinate (440 mg, 567.77 μmol, 1 equiv.), LiOH·H₂O (71.47 mg, 1.70 mmol, 3 equiv.) in THE (1.2 mL) and H₂O (0.4 mL) was stirred at 25° C. for 1.5 hours. The mixture was concentrated and redissolved by H₂O (2 mL), and then 1M HCl was added to adjust the pH to 2. The residue was filtered and dried to give 3-((1r,4r)-4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)cyclohexyl)propanoic acid (380 mg, 508.7 μmol, 89.6% yield) was obtained as a gray solid.

### Step H. Procedure for Preparation of tert-butyl 6-(8-(benzo[d]thiazol-2-ylcarbamoyl)-3,4-dihydroisoquinolin-2(1H)-yl)-3-(3-(((1r,4r)-4-(3-((3-(2,6-dioxopiperidin-3-yl)-1-methyl-1H-indazol-7-yl)amino)-3-oxopropyl)cyclohexyl)oxy)-2-methylphenyl)picolinate

A mixture of 3-((1r,4r)-4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)cyclohexyl)propanoic acid (150 mg, 200.83 μmol, 1 equiv.), 3-(7-amino-1-methyl-indazol-3-yl)piperidine-2,6-dione (57.06 mg, 220.91 μmol, 1.1 equiv.), HATU (76.36 mg, 200.83 μmol, 1 equiv.), and DIPEA (77.86 mg, 602.48 μmol, 104.94 μL, 3 equiv.) in DMF (0.5 mL) was stirred at 25° C. for 12 hours. The reaction mixture was diluted with H₂O (1 mL) and extracted with ethyl acetate (1 mL×3). The combined organic layers were washed with H₂O (1 mL×3), dried over anhydrous Na₂SO₄, filtered, and concentrated under reduced pressure to give tert-butyl 6-(8-(benzo[d]thiazol-2-ylcarbamoyl)-3,4-dihydroisoquinolin-2(1H)-yl)-3-(3-(((1r,4r)-4-(3-((3-(2,6-dioxopiperidin-3-yl)-1-methyl-1H-indazol-7-yl)amino)-3-oxopropyl)cyclohexyl)oxy)-2-methylphenyl)picolinate (130 mg, crude) as a white solid.

### Step L Procedure for preparation of 6-(8-(benzo[d]thiazol-2-ylcarbamoyl)-3,4-dihydroisoquinolin-2(1H)-yl)-3-(3-(((1r,4r)-4-(3-((3-(2,6-dioxopiperidin-3-yl)-1-methyl-1H-indazol-7-yl)amino)-3-oxopropyl)cyclohexyl)oxy)-2-methylphenyl)picolinic acid

Tert-butyl 6-(8-(benzo[d]thiazol-2-ylcarbamoyl)-3,4-dihydroisoquinolin-2(1H)-yl)-3-(3-(((1r,4r)-4-(3-((3-(2,6-dioxopiperidin-3-yl)-1-methyl-1H-indazol-7-yl)amino)-3-oxopropyl)cyclohexyl)oxy)-2-methylphenyl)picolinate (130 mg, 131.69 μmol, 1 equiv.) in TFA (0.5 mL) and DCM (0.5 mL) was stirred at 40° C. for 6 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1r,4r)-4-(3-((3-(2,6-dioxopiperidin-3-yl)-1-methyl-1H-indazol-7-yl)amino)-3-oxopropyl)cyclohexyl)oxy)-2-methylphenyl)picolinic acid (47.2 mg, 50.2 μmol, 38.1% yield, 99.03% purity) as a white solid.

MS (ESI) m/z: 931.7 [M+H]⁺.

¹H NMR (400 MHz, DMSO-d₆) δ=10.88 (s, 1H), 9.88 (s, 1H), 8.03 (d, J=7.6 Hz, 1H), 7.79 (d, J=8.0 Hz, 1H), 7.61 (dd, J=7.6, 12.0 Hz, 2H), 7.52-7.30 (m, 6H), 7.16-7.02 (m, 3H), 6.96 (dd, J=8.8, 13.2 Hz, 2H), 6.62 (d, J=7.2 Hz, 1H), 4.98 (s, 2H), 4.37 (dd, J=4.4, 8.8 Hz, 1H), 4.27-4.16 (m, 2H), 4.05 (s, 3H), 3.91 (s, 2H), 3.03 (s, 2H), 2.74-2.60 (m, 2H), 2.47-2.32 (m, 4H), 2.20-2.06 (m, 3H), 1.88 (s, 3H), 1.59 (d, J=6.4 Hz, 2H), 1.45-1.30 (m, 3H), 1.21-1.13 (m, 2H).

### Example 132. Preparation of Compound 128

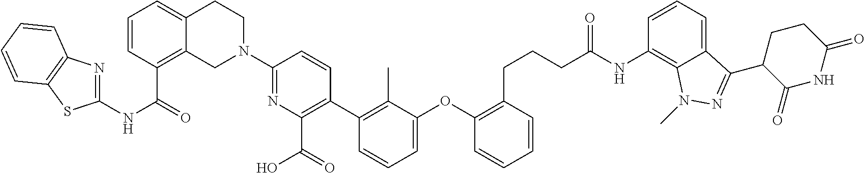 [SMILES: Cc1c(Oc2ccccc2CCCC(=O)Nc2cccc3c(C4CCC(=O)NC4=O)nn(C)c23)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(2-(4-((3-(2,6-dioxopiperidin-3-yl)-1-methyl-1H-indazol-7-yl)amino)-4-oxobutyl)phenoxy)-2-methylphenyl)picolinic acid

### Step A. Procedure for Preparation of ethyl 4-(2-(3-bromo-2-methylphenoxy)phenyl)butanoate

A mixture of ethyl 4-(2-hydroxyphenyl)butanoate (1.8 g, 8.64 mmol, 1 equiv.), 1,3-dibromo-2-methylbenzene (4.32 g, 17.29 mmol, 2 equiv.), 2,2,6,6-tetramethylheptane-3,5-dione (398.19 mg, 2.16 mmol, 444.90 μL, 0.25 equiv.), CuI (823.06 mg, 4.32 mmol, 0.5 equiv.), and Cs₂CO₃ (3.38 g, 10.37 mmol, 1.2 equiv.) in NMP (20 mL) was degassed and purged with N₂, and the mixture was stirred at 120° C. for 12 hours under N₂ atmosphere. The reaction mixture was concentrated under reduced pressure (3 g, crude) to give a residue. The residue was purified by flash silica gel chromatography (Eluent of 0-1% ethyl acetate/petroleum ether) to give ethyl 4-(2-(3-bromo-2-methylphenoxy)phenyl)butanoate (1.9 g, 5.0 mmol, 63.3% yield) as a blackish green oil.

¹H NMR (400 MHz, DMSO-d₆) δ=7.37 (d, J=8.0 Hz, 1H), 7.31 (d, J=7.2 Hz, 1H), 7.23-7.16 (m, 1H), 7.14-7.06 (m, 2H), 6.74-6.66 (m, 2H), 3.99 (q, J=7.2 Hz, 2H), 2.60 (t, J=7.6 Hz, 2H), 2.33-2.26 (m, 5H), 1.88-1.78 (m, 2H), 1.12 (t, J=7.2 Hz, 3H)

### Step B. Procedure for Preparation of tert-butyl 6-(8-(benzo[d]thiazol-2-ylcarbamoyl)-3,4-dihydroisoquinolin-2(1H)-yl)-3-(3-(2-(4-ethoxy-4-oxobutyl)phenoxy)-2-methylphenyl)picolinate

Ethyl 4-(2-(3-bromo-2-methylphenoxy)phenyl)butanoate (190.93 mg, 506.09 μmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (310 mg, 506.09 μmol, 1 equiv.), K₂CO₃ (209.83 mg, 1.52 mmol, 3 equiv.), and Ad₂nBuP Pd G₃(cataCXium® A Pd G₃) (36.86 mg, 50.61 μmol, 0.1 equiv.) were taken up into a microwave tube in dioxane (5 mL). The sealed tube was heated at 100° C. for 120 minutes under microwave. The reaction mixture was diluted with water (20 mL) and extracted with EtOAc 25 mL×3. The combined organic layers were washed with brine (15 mL×3), dried over anhydrous sodium sulfate, filtered, and concentrated under reduced pressure to give a residue. The residue was purified by prep-TLC (SiO₂, petroleum ether:ethyl acetate=1:1) to give compound tert-butyl 6-(8-(benzo[d]thiazol-2-ylcarbamoyl)-3,4-dihydroisoquinolin-2(1H)-yl)-3-(3-(2-(4-ethoxy-4-oxobutyl)phenoxy)-2-methylphenyl)picolinate (250 mg, 319.31 μmol, 63.09% yield) as a yellow solid.

MS (ESI) m/z: 783.2 [M+H]⁺.

### Step C. Procedure for Preparation of 4-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)phenyl)butanoic acid

A mixture of tert-butyl 6-(8-(benzo[d]thiazol-2-ylcarbamoyl)-3,4-dihydroisoquinolin-2(1H)-yl)-3-(3-(2-(4-ethoxy-4-oxobutyl)phenoxy)-2-methylphenyl)picolinate (250 mg, 319.31 μmol, 1 equiv.) and LiOH H₂O (40.20 mg, 957.92 μmol, 3 equiv.) in THE (2 mL) and H₂O (0.5 mL) was degassed and purged with N₂ and stirred at 25° C. for 1 hour under N₂ atmosphere. The reaction mixture was diluted with water (20 mL) and extracted with EtOAc (25 mL×3). The combined organic layers were washed with brine (15 mL×3), dried over anhydrous sodium sulfate, filtered and concentrated under reduced pressure to give 4-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)phenyl)butanoic acid (150 mg, crude) as a yellow solid.

MS (ESI) m/z: 755.5 [M+H]⁺

### Step D. Procedure for Preparation of tert-butyl 6-(8-(benzo[d]thiazol-2-ylcarbamoyl)-3,4-dihydroisoquinolin-2(1H)-yl)-3-(3-(2-(4-((3-(2,6-dioxopiperidin-3-yl)-1-methyl-1H-indazol-7-yl)amino)-4-oxobutyl)phenoxy)-2-methylphenyl)picolinate

A mixture of 4-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)phenyl)butanoic acid (90 mg, 119.22 μmol, 1 equiv.), 3-(7-amino-1-methyl-1H-indazol-3-yl)piperidine-2,6-dione (33.87 mg, 131.14 μmol, 1.1 equiv.), DIEA (46.23 mg, 357.67 μmol, 62.30 uL, 3 equiv.), and HATU (54.40 mg, 143.07 μmol, 1.2 equiv.) in DMF (1.2 mL) was degassed and purged with N₂ and stirred at 25° C. for 1 hour under N₂ atmosphere. The mixture was concentrated under reduced pressure to give tert-butyl 6-(8-(benzo[d]thiazol-2-ylcarbamoyl)-3,4-dihydroisoquinolin-2(1H)-yl)-3-(3-(2-(4-((3-(2,6-dioxopiperidin-3-yl)-1-methyl-1H-indazol-7-yl)amino)-4-oxobutyl)phenoxy)-2-methylphenyl)picolinate (100 mg, 100.5 μmol, 84.3% yield) as a yellow oil.

MS (ESI) m/z: 995.2 [M+H]⁺.

### Step E. Procedure for Preparation of 6-(8-(benzo[d]thiazol-2-ylcarbamoyl)-3,4-dihydroisoquinolin-2(1H)-yl)-3-(3-(2-(4-((3-(2,6-dioxopiperidin-3-yl)-1-methyl-1H-indazol-7-yl)amino)-4-oxobutyl)phenoxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(3-(2-(4-((3-(2,6-dioxopiperidin-3-yl)-1-methyl-1H-indazol-7-yl)amino)-4-oxobutyl)phenoxy)-2-methylphenyl)picolinate (100 mg, 100.49 μmol, 1 equiv.) in TFA (1 mL) and DCM (1 mL) was degassed and purged with N₂ and stirred at 35° C. for 1 hour under N₂ atmosphere. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(2-(4-((3-(2,6-dioxopiperidin-3-yl)-1-methyl-1H-indazol-7-yl)amino)-4-oxobutyl)phenoxy)-2-methylphenyl)picolinic acid (24.52 mg, 25.9 μmol, 25.8% yield, 99.4% purity) as a yellow solid.

MS (ESI) m/z: 939.4 [M+H]⁺

¹H NMR (400 MHz, DMSO-d₆) δ=12.86-12.68 (m, 1H), 10.89 (s, 1H), 9.86 (s, 1H), 8.03 (d, J=7.6 Hz, 1H), 7.79 (d, J=7.6 Hz, 1H), 7.63 (d, J=7.6 Hz, 1H), 7.57 (d, J=6.4 Hz, 1H), 7.55-7.27 (m, 7H), 7.20-7.14 (m, 2H), 7.09-7.02 (m, 3H), 6.98 (d, J=8.4 Hz, 1H), 6.88 (d, J=7.2 Hz, 1H), 6.77 (d, J=8.0 Hz, 1H), 6.63 (d, J=8.0 Hz, 1H), 5.00 (s, 2H), 4.39-4.32 (m, 1H), 3.98 (s, 3H), 3.93 (t, J=5.6 Hz, 2H), 3.03 (t, J=5.6 Hz, 2H), 2.75 (t, J=7.6 Hz, 2H), 2.66-2.59 (m, 2H), 2.45 (t, J=7.2 Hz, 2H), 2.38-2.31 (m, 1H), 2.19-2.12 (m, 1H), 2.04-1.94 (m, 5H).

### Example 133. Preparation of Compound 129

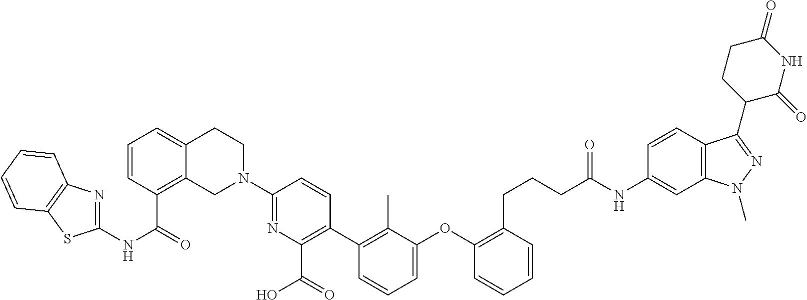 [SMILES: Cc1c(Oc2ccccc2CCCC(=O)Nc2ccc3c(C4CCC(=O)NC4=O)nn(C)c3c2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(2-(4-((3-(2,6-dioxopiperidin-3-yl)-1-methyl-1H-indazol-6-yl)amino)-4-oxobutyl)phenoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(2-(4-((3-(2,6-dioxopiperidin-3-yl)-1-methyl-1H-indazol-6-yl)amino)-4-oxobutyl)phenoxy)-2-methylphenyl)picolinate

A mixture of 4-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)phenyl)butanoic acid (100 mg, 105.98 μmol, 80% purity, 1 equiv.), 3-(6-amino-1-methyl-1H-indazol-3-yl)piperidine-2,6-dione (38.32 mg, 148.37 μmol, 1.4 equiv.), DIEA (41.09 mg, 317.93 μmol, 55.38 μL, 3 equiv.), and HATU (48.35 mg, 127.17 μmol, 1.2 equiv.) in DMF (1.4 mL) was degassed and purged with N₂ and stirred at 25° C. for 2 hours under N₂ atmosphere. The reaction mixture was diluted with water (20 mL) and extracted with DCM (25 mL×3). The combined organic layers were washed with brine (15 mL×3), dried over anhydrous sodium sulfate, filtered and concentrated under reduced pressure to give a residue. The residue was tert-butyl 6-(8-(benzo[d]thiazol-2-ylcarbamoyl)-3,4-dihydroisoquinolin-2(1H)-yl)-3-(3-(2-(4-((3-(2,6-dioxopiperidin-3-yl)-1-methyl-1H-indazol-6-yl)amino)-4-oxobutyl)phenoxy)-2-methylphenyl)picolinate (100 mg, crude) as a brown oil.

MS (ESI) m/z: 995.4 [M+H]⁺.

### Step B. Procedure for Preparation of 6-(8-(benzo[d]thiazol-2-ylcarbamoyl)-3,4-dihydroisoquinolin-2(1H)-yl)-3-(3-(2-(4-((3-(2,6-dioxopiperidin-3-yl)-1-methyl-1H-indazol-6-yl)amino)-4-oxobutyl)phenoxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(3-(2-(4-((3-(2,6-dioxopiperidin-3-yl)-1-methyl-1H-indazol-6-yl)amino)-4-oxobutyl)phenoxy)-2-methylphenyl)picolinate (100 mg, 100.49 μmol, 1 equiv.) in TFA (0.5 mL) and DCM (0.5 mL) was degassed and purged with N₂ and stirred at 40° C. for 1 hour under N₂ atmosphere. The reaction mixture was filtered and concentrated under reduced pressure to give a residue. The crude product was purified by prep-HPLC to give 6-(8-(benzo[d]thiazol-2-ylcarbamoyl)-3,4-dihydroisoquinolin-2(1H)-yl)-3-(3-(2-(4-((3-(2,6-dioxopiperidin-3-yl)-1-methyl-1H-indazol-6-yl)amino)-4-oxobutyl)phenoxy)-2-methylphenyl)picolinic acid (20.0 mg, 20.4 μmol, 20.3% yield, 95.7% purity) as a yellow solid.

MS (ESI) m/z: 939.5 [M+H]⁺

¹H NMR (400 MHz, DMSO-d₆) δ=12.85-12.58 (m, 2H), 10.84 (s, 1H), 10.06 (s, 1H), 8.08-8.00 (m, 2H), 7.79 (d, J=8.0 Hz, 1H), 7.65-7.56 (m, 2H), 7.51-7.42 (m, 3H), 7.40-7.30 (m, 3H), 7.18-7.12 (m, 2H), 7.09-7.02 (m, 2H), 7.00-6.95 (m, 1H), 6.86 (d, J=7.6 Hz, 1H), 6.77 (d, J=8.0 Hz, 1H), 6.65-6.59 (m, 1H), 5.00 (s, 2H), 4.34-4.25 (m, 1H), 3.96-3.90 (m, 2H), 3.88 (s, 2H), 3.04 (t, J=5.6 Hz, 2H), 2.75-2.57 (m, 4H), 2.41 (t, J=7.6 Hz, 3H), 2.33-2.14 (m, 3H), 1.99-1.93 (m, 4H)

### Example 134. Preparation of Compound 132

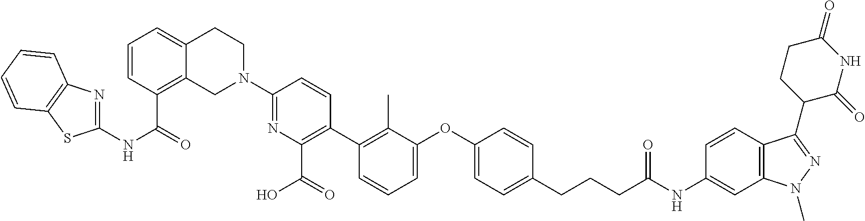 [SMILES: Cc1c(Oc2ccc(CCCC(=O)Nc3ccc4c(C5CCC(=O)NC5=O)nn(C)c4c3)cc2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(4-(4-((3-(2,6-dioxopiperidin-3-yl)-1-methyl-1H-indazol-6-yl)amino)-4-oxobutyl)phenoxy)-2-methylphenyl)picolinic acid

### Step A. Procedure for Preparation of methyl 4-(4-hydroxyphenyl)butanoate

To a solution of 4-(4-hydroxyphenyl)butanoic acid (2 g, 11.10 mmol, 1 equiv.) in MeOH (25 mL) was added H₂SO₄ (23.55 g, 240.13 mmol, 12.8 mL, 21.64 equiv.). The mixture was stirred at 25° C. for 12 hours. The reaction mixture was diluted with H₂O (50 mL) and extracted with ethyl acetate (50 mL×2). The combined organic layers were washed with brine (20 mL×2), dried over Na₂SO₄, filtered, and concentrated under reduced pressure to give a residue. The residue was purified by flash silica gel chromatography (Eluent of 0˜20% ethyl acetate/petroleum ether) to give methyl 4-(4-hydroxyphenyl)butanoate (1.7 g, 7.8 mmol, 70.9% yield, 90% purity) as a colorless oil.

¹H NMR (400 MHz, CDCl₃) δ=7.04 (d, J=6.8 Hz, 2H), 6.78-6.75 (m, 2H), 3.69-3.67 (m, 3H), 2.58 (t, J=7.2 Hz, 2H), 2.33 (dt, J=1.6, 6.0 Hz, 2H), 1.98-1.91 (m, 2H).

### Step B. Procedure for Preparation of methyl 4-(4-(3-bromo-2-methylphenoxy)phenyl)butanoate

A mixture of methyl 4-(4-hydroxyphenyl)butanoate (500 mg, 2.57 mmol, 1 equiv.), 1,3-dibromo-2-methylbenzene (1.93 g, 7.72 mmol, 3 equiv.), CuI (245.14 mg, 1.29 mmol, 0.5 equiv.), Cs₂CO₃ (1.01 g, 3.09 mmol, 1.2 equiv.), and 2,2,6,6-tetramethylheptane-3,5-dione (118.60 mg, 643.58 μmol, 132.51 uL, 0.25 equiv.) in NMP (20 mL) was degassed and purged with N₂ and stirred at 120° C. for 12 hours under N₂ atmosphere. The reaction mixture was diluted with H₂O (30 mL) and extracted with ethyl acetate (20 mL×4). The combined organic layers were washed with brine (15 mL×2), dried over Na₂SO₄, filtered, and concentrated under reduced pressure to give a residue. The residue was purified by flash silica gel chromatography (Eluent of 0-7% ethyl acetate/petroleum ether) to give methyl 4-(4-(3-bromo-2-methylphenoxy)phenyl)butanoate (900 mg, 2.2 mmol, 86.6% yield, 90% purity) as a yellow oil.

¹H NMR (400 MHz, CDCl₃) δ=7.36-7.32 (m, 1H), 7.13 (d, J=8.4 Hz, 2H), 7.01 (t, J=8.4 Hz, 1H), 6.88-6.82 (m, 3H), 3.68 (s, 3H), 2.63 (t, J=7.6 Hz, 2H), 2.38-2.33 (m, 5H), 1.95 (q, J=7.6 Hz, 2H).

### Step C. Procedure for Preparation of tert-butyl 6-(8-(benzo[d]thiazol-2-ylcarbamoyl)-3,4-dihydroisoquinolin-2(1H)-yl)-3-(3-(4-(4-methoxy-4-oxobutyl)phenoxy)-2-methylphenyl)picolinate

A mixture of tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (850 mg, 1.39 mmol, 1 equiv.) and methyl 4-(4-(3-bromo-2-methylphenoxy)phenyl)butanoate (672.08 mg, 1.67 mmol, 90% purity, 1.2 equiv.) in 1,4-dioxane (10 mL) was added Ad₂nBuP Pd G₃(cataCXium® A Pd G₃) (101.06 mg, 138.77 μmol, 0.1 equiv.) and KF (1.5 M, 2.78 mL, 3 equiv.). After addition, the mixture was degassed and purged with N₂ and stirred at 100° C. for 2 hours under microwave. The reaction mixture was filtered and concentrated under reduced pressure to give a residue. The residue was purified by flash silica gel chromatography (Eluent of 0˜30% Dichloromethane/Methanol) to give tert-butyl 6-(8-(benzo[d]thiazol-2-ylcarbamoyl)-3,4-dihydroisoquinolin-2(1H)-yl)-3-(3-(4-(4-methoxy-4-oxobutyl)phenoxy)-2-methylphenyl)picolinate (520 mg, 635.7 μmol, 45.8% yield, 94% purity) as a yellow solid.

MS (ESI) m/z: 769.9 [M+H]⁺.

### Step D. Procedure for Preparation of 4-(4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy) phenyl)butanoic acid

To a solution of tert-butyl 6-(8-(benzo[d]thiazol-2-ylcarbamoyl)-3,4-dihydroisoquinolin-2(1H)-yl)-3-(3-(4-(4-methoxy-4-oxobutyl)phenoxy)-2-methylphenyl)picolinate (520 mg, 635.70 μmol, 94% purity, 1 equiv.) in THE (5 mL) was added LiOH·H₂O (80.03 mg, 1.91 mmol, 3 equiv.) and H₂O (1.25 mL) at 25° C. The mixture was stirred at 25° C. for 12 hours. The reaction mixture was concentrated under reduced pressure to remove THF. The aqueous phase was adjusted to pH=4˜5 with 1M HCl. The reaction mixture was filtered and diluted in ethyl acetate. The combined organic layers were dried over Na₂SO₄, filtered, and concentrated under reduced pressure to give 4-(4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy) phenyl)butanoic acid (500 mg, crude) as a yellow solid.

MS (ESI) m/z: 755.5 [M+H]⁺.

### Step E. Procedure for Preparation of tert-butyl 6-(8-(benzo[d]thiazol-2-ylcarbamoyl)-3,4-dihydroisoquinolin-2(1H)-yl)-3-(3-(4-(4-((3-(2,6-dioxopiperidin-3-yl)-1-methyl-1H-indazol-6-yl)amino)-4-oxobutyl)phenoxy)-2-methylphenyl)picolinate

A mixture of 4-(4-(3-(6-(8-(benzo[d]thiazol-2-ylcarbamoyl)-3, 4-dihydroisoquinolin-2(1H)-yl)-2-(tert-butoxycarbonyl)pyridin-3-yl)-2-methylphenoxy)phenyl)butanoic acid (100 mg, 132.47 μmol, 1 equiv.), HATU (55.41 mg, 145.72 μmol, 1.1 equiv.), and DIEA (51.36 mg, 397.41 μmol, 69.22 μL, 3 equiv.) in DMF (1 mL) was added 3-(6-amino-1-methyl-1H-indazol-3-yl)piperidine-2,6-dione (34.21 mg, 132.47 μmol, 1 equiv.), and then the mixture was stirred at 25° C. for 2 hours. The reaction mixture was treated with water (2 mL), filtered, washed with CH₂Cl₂ (10 mL), and concentrated under the reduced pressure to give tert-butyl 6-(8-(benzo[d]thiazol-2-ylcarbamoyl)-3,4-dihydroisoquinolin-2(1H)-yl)-3-(3-(4-(4-((3-(2,6-dioxopiperidin-3-yl)-1-methyl-1H-indazol-6-yl)amino)-4-oxobutyl)phenoxy)-2-methylphenyl)picolinate (103 mg, 83.8 μmol, 63.3% yield, 81% purity) as a yellow solid.

MS (ESI) m/z: 995.5 [M+H]⁺.

### Step F. Procedure for Preparation of 6-(8-(benzo[d]thiazol-2-ylcarbamoyl)-3,4-dihydroisoquinolin-2(1H)-yl)-3-(3-(4-(4-((3-(2,6-dioxopiperidin-3-yl)-1-methyl-1H-indazol-6-yl)amino)-4-oxobutyl)phenoxy)-2-methylphenyl)picolinic acid

tert-Butyl 6-(8-(benzo[d]thiazol-2-ylcarbamoyl)-3,4-dihydroisoquinolin-2(1H)-yl)-3-(3-(4-(4-((3-(2,6-dioxopiperidin-3-yl)-1-methyl-1H-indazol-6-yl)amino)-4-oxobutyl)phenoxy)-2-methylphenyl)picolinate (100 mg, 100.49 μmol, 1 equiv.) in CH₂Cl₂ (1.5 mL) and TFA (0.5 mL) was stirred at 40° C. for 2 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(4-(4-((3-(2,6-dioxopiperidin-3-yl)-1-methyl-1H-indazol-6-yl)amino)-4-oxobutyl)phenoxy)-2-methylphenyl)picolinic acid (28.7 mg, 29.9 μmol, 29.7% yield, 97.7% purity) as a white solid.

MS (ESI) m/z: 939.7 [M+H]⁺.

¹H NMR (400 MHz, DMSO-d₆) δ=10.88 (s, 1H), 10.08 (s, 1H), 8.08 (s, 1H), 8.03 (d, J=7.6 Hz, 1H), 7.79 (d, J=8.0 Hz, 1H), 7.64-7.58 (m, 2H), 7.52 (d, J=8.6 Hz, 1H), 7.49-7.44 (m, 2H), 7.39-7.33 (m, 2H), 7.24-7.11 (m, 4H), 7.07 (d, J=8.8 Hz, 1H), 6.99 (d, J=8.4 Hz, 1H), 6.89 (d, J=7.6 Hz, 1H), 6.83 (d, J=8.4 Hz, 3H), 4.99 (s, 2H), 4.30 (dd, J=4.8, 9.6 Hz, 1H), 3.96-3.92 (m, 2H), 3.90 (s, 3H), 3.03 (t, J=5.4 Hz, 2H), 2.70-2.64 (m, 2H), 2.40-2.35 (m, 2H), 2.35 (s, 2H), 2.24-2.08 (m, 2H), 1.93-1.88 (m, 5H)

### Example 135. Preparation of Compound 134

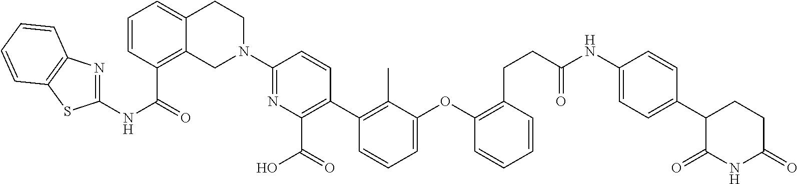 [SMILES: Cc1c(Oc2ccccc2CCC(=O)Nc2ccc(C3CCC(=O)NC3=O)cc2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(2-(3-((4-(2,6-dioxopiperidin-3-yl)phenyl)amino)-3-oxopropyl)phenoxy)-2-methylphenyl)picolinic acid

### Step A. Procedure for Preparation of ethyl 3-(2-(3-bromo-2-methylphenoxy)phenyl)propanoate

A mixture of ethyl 3-(2-hydroxyphenyl)propanoate (2 g, 10.30 mmol, 1 equiv.), 1,3-dibromo-2-methylbenzene (5.15 g, 20.59 mmol, 2 equiv.), CuI (980.56 mg, 5.15 mmol, 0.5 equiv.), Cs₂CO₃ (4.03 g, 12.36 mmol, 1.2 equiv.), and 2,2,6,6-tetramethylheptane-3,5-dione (474.38 mg, 2.57 mmol, 530.04 uL, 0.25 equiv.) in NMP (20 mL) was degassed and purged with N₂ and stirred at 120° C. for 12 hours under N₂ atmosphere. The reaction mixture was diluted with H₂O (50 mL) and extracted with EtOAc (100 mL×3). The combined organic layers were washed with saturated NaHCO₃ solution (100 mL×3), dried over Na₂SO₄, filtered, and concentrated under reduced pressure to give a residue. The residue was purified by flash silica gel chromatography (Eluent of 0˜10% ethyl acetate/petroleum ether) to give ethyl 3-(2-(3-bromo-2-methylphenoxy)phenyl)propanoate (1.7 g, crude) as a green oil.

¹H NMR (400 MHz, CD₃OD) δ=7.37-7.27 (m, 2H), 7.22-7.13 (m, 1H), 7.10-7.01 (m, 2H), 6.75-6.63 (m, 2H), 4.11-4.04 (m, 2H), 2.96 (t, J=7.6 Hz, 2H), 2.68-2.60 (m, 2H), 2.41-2.34 (m, 3H), 1.21-1.17 (m, 3H).

### Step B. Procedure for Preparation of tert-butyl 6-(8-(benzo[d]thiazol-2-ylcarbamoyl)-3,4-dihydroisoquinolin-2(1H)-yl)-3-(3-(2-(3-ethoxy-3-oxopropyl)phenoxy)-2-methylphenyl)picolinate

A mixture of ethyl 3-(2-(3-bromo-2-methylphenoxy)phenyl)propanoate (400 mg, 1.10 mmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (674.53 mg, 1.10 mmol, 1 equiv.), di-tert-butyl(cyclopentyl)phosphane; dichloropalladium; iron (71.77 mg, 110.12 μmol, 0.1 equiv.), and K₂CO₃ (1.5 M, 2.20 mL, 3 equiv.) in dioxane (5.5 mL) was degassed and purged with N₂ and stirred at 80° C. for 12 hours under N₂ atmosphere. The reaction mixture was diluted with H₂O (10 mL) and extracted with solvent (20 mL×3). The combined organic layers were washed with brine (60 mL), dried over Na₂SO₄, filtered, and concentrated under reduced pressure to give a residue. The residue was purified by flash silica gel chromatography (Eluent of 0˜35% ethyl acetate/petroleum ether) to give tert-butyl 6-(8-(benzo[d]thiazol-2-ylcarbamoyl)-3,4-dihydroisoquinolin-2(1H)-yl)-3-(3-(2-(3-ethoxy-3-oxopropyl)phenoxy)-2-methylphenyl)picolinate (520 mg, 517.8 μmol, 47.0% yield, 76.5% purity) as a yellow oil.

¹H NMR (400 MHz, CDCl₃) δ=7.34 (d, J=8.0 Hz, 1H), 7.17-7.13 (m, 2H), 7.01 (t, J=8.0 Hz, 1H), 6.86-6.81 (m, 3H), 3.69 (t, J=6.4 Hz, 2H), 2.72-2.68 (m, 2H), 2.36 (s, 3H), 1.92-1.87 (m, 2H)

### Step C. Procedure for Preparation of 3-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)phenyl)propanoic acid

To a solution of tert-butyl 6-(8-(benzo[d]thiazol-2-ylcarbamoyl)-3,4-dihydroisoquinolin-2(1H)-yl)-3-(3-(2-(3-ethoxy-3-oxopropyl)phenoxy)-2-methylphenyl)picolinate (520 mg, 517.89 μmol, 76.58% purity, 1 equiv.) in THE (3 mL) was added LiOH·H₂O (1 M, 2.5 mL, 4.83 equiv.). The mixture was stirred at 25° C. for 12 hours. The mixture was concentrated under reduced pressure to remove THF, treated with HCl (1 M) to pH=6, and filtered to give 3-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)phenyl)propanoic acid (380 mg, crude) as a yellow solid.

¹H NMR (400 MHz, CDCl₃) δ=9.87-9.80 (m, 1H), 7.38-7.31 (m, 1H), 7.16-7.13 (m, 2H), 7.05-6.97 (m, 1H), 6.86-6.81 (m, 3H), 2.98-2.92 (m, 2H), 2.81-2.76 (m, 2H), 2.34 (s, 3H)

### Step D. Procedure for Preparation of tert-butyl 6-(8-(benzo[d]thiazol-2-ylcarbamoyl)-3,4-dihydroisoquinolin-2(1H)-yl)-3-(3-(2-(3-((4-(2,6-dioxopiperidin-3-yl)phenyl)amino)-3-oxopropyl)phenoxy)-2-methylphenyl)picolinate

To a solution of 3-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)phenyl)propanoic acid (90 mg, 121.48 μmol, 1 equiv.) in DMF (1 mL) was added HATU (69.29 mg, 182.22 μmol, 1.5 equiv.), DIEA (47.10 mg, 364.44 μmol, 63.48 μL, 3 equiv.), and 3-(4-aminophenyl)piperidine-2,6-dione (37.21 mg, 182.22 μmol, 1.5 equiv.). The mixture was stirred at 40° C. for 1 hour. The reaction was added into H₂O (5 mL) and filtered to give tert-butyl 6-(8-(benzo[d]thiazol-2-ylcarbamoyl)-3,4-dihydroisoquinolin-2(1H)-yl)-3-(3-(2-(3-((4-(2,6-dioxopiperidin-3-yl)phenyl)amino)-3-oxopropyl)phenoxy)-2-methylphenyl)picolinate (100 mg, crude) as a yellow solid.

MS (ESI) m/z: 927.7 [M+H]⁺

### Step E. Procedure for Preparation of 6-(8-(benzo[d]thiazol-2-ylcarbamoyl)-3,4-dihydroisoquinolin-2(1H)-yl)-3-(3-(2-(3-((4-(2,6-dioxopiperidin-3-yl)phenyl)amino)-3-oxopropyl)phenoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(2-(3-((4-(2,6-dioxopiperidin-3-yl)phenyl)amino)-3-oxopropyl)phenoxy)-2-methylphenyl)picolinate (100 mg, 107.87 μmol, 1 equiv.) in DCM (0.5 mL) was added TFA (770.00 mg, 6.75 mmol, 0.5 mL, 62.61 equiv.). The mixture was stirred at 25° C. for 12 hours. The mixture was concentrated under reduced pressure to remove DCM. The residue was purified by prep-HPLC to give 6-(8-(benzo[d]thiazol-2-ylcarbamoyl)-3,4-dihydroisoquinolin-2(1H)-yl)-3-(3-(2-(3-((4-(2,6-dioxopiperidin-3-yl)phenyl)amino)-3-oxopropyl)phenoxy)-2-methylphenyl)picolinic acid (23.9 mg, 26.7 μmol, 24.8% yield, 97.58% purity) as a yellow solid.

MS (ESI) m/z: 871.4 [M+H]⁺

¹H NMR (400 MHz, DMSO-d₆) δ=12.91-12.82 (m, 1H), 12.80-12.58 (m, 1H), 10.80 (s, 1H), 9.94-9.89 (m, 1H), 8.03 (d, J=8.0 Hz, 1H), 7.82-7.76 (m, 1H), 7.64-7.60 (m, 1H), 7.55-7.50 (m, 3H), 7.48-7.43 (m, 2H), 7.40-7.31 (m, 3H), 7.20-7.11 (m, 4H), 7.04-6.99 (m, 2H), 6.88 (d, J=6.8 Hz, 1H), 6.81 (d, J=7.6 Hz, 1H), 6.58 (d, J=8.0 Hz, 1H), 5.00 (s, 2H), 3.95-3.91 (m, 2H), 3.80-3.77 (m, 1H), 3.07-3.01 (m, 4H), 3.01-2.95 (m, 4H), 2.70-2.69 (m, 1H), 2.17-2.12 (m, 1H), 1.95 (s, 3H)

### Example 136. Preparation of Compound 139

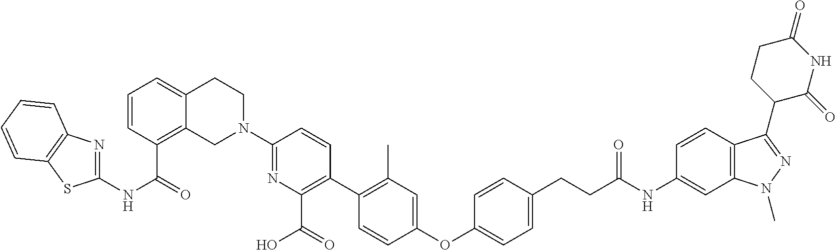 [SMILES: Cc1cc(Oc2ccc(CCC(=O)Nc3ccc4c(C5CCC(=O)NC5=O)nn(C)c4c3)cc2)ccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(4-(4-(3-((3-(2,6-dioxopiperidin-3-yl)-1-methyl-1H-indazol-6-yl)amino)-3-oxopropyl)phenoxy)-2-methylphenyl)picolinic acid

### Step A. Procedure for Preparation of methyl 3-(4-(4-bromo-3-methylphenoxy)phenyl)propanoate

To a solution of (4-bromo-3-methylphenyl)boronic acid (2.00 g, 9.31 mmol, 1 equiv.) in DCM (20 mL) was added methyl 3-(4-hydroxyphenyl)propanoate (2.01 g, 11.17 mmol, 1.2 equiv.), TEA (941.94 mg, 9.31 mmol, 1.30 mL, 1 equiv.), 4A MS (20 mg, 465.44 umol), and Cu(OAc)₂ (1.69 g, 9.31 mmol, 1 equiv.) at 25° C. The reaction mixture was purged with O₂ and stirred at 25° C. for 40 hours under O₂ (15 Psi). The reaction mixture was filtered and concentrated under reduced pressure to give a residue. The residue was purified by flash silica gel chromatography (Eluent of 0˜10% Ethylacetate/petroleum ether). Methyl 3-(4-(4-bromo-3-methylphenoxy)phenyl)propanoate (360 mg, 1.0 mmol, 11.0% yield) was obtained as an colorless oil.

¹H NMR (400 MHz, CDCl₃) δ=7.45 (d, J=8.8 Hz, 1H), 7.17 (d, J=8.4 Hz, 2H), 6.95-6.90 (m, 2H), 6.89 (d, J=2.8 Hz, 1H), 6.70 (dd, J=2.8, 8.8 Hz, 1H), 3.69 (s, 3H), 2.95 (t, J=8.0 Hz, 2H), 2.68-2.60 (m, 2H), 2.36 (s, 3H)

### Step B. Procedure for Preparation of tert-butyl 6-(8-(benzo[d]thiazol-2-ylcarbamoyl)-3,4-dihydroisoquinolin-2(1H)-yl)-3-(4-(4-(3-methoxy-3-oxopropyl)phenoxy)-2-methylphenyl)picolinate

Methyl 3-(4-(4-bromo-3-methylphenoxy)phenyl)propanoate (330 mg, 944.97 μmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (578.84 mg, 944.97 μmol, 1 equiv.), KF (1.5 M, 1.89 mL, 3 equiv.), and [2-(2-aminophenyl)phenyl]palladium(1+); bis(1-adamantyl)-butyl-phosphane; methanesulfonate (137.64 mg, 188.99 μmol, 0.2 equiv.) were taken up into a microwave tube in dioxane (3.3 mL). The sealed tube was heated at 100° C. for 1 hour under microwave. The reaction mixture was concentrated under reduced pressure to give a residue. The residue was purified by flash silica gel chromatography (Eluent of 0˜20% ethyl acetate/petroleum ether). tert-Butyl 6-(8-(benzo[d]thiazol-2-ylcarbamoyl)-3,4-dihydroisoquinolin-2(1H)-yl)-3-(4-(4-(3-methoxy-3-oxopropyl)phenoxy)-2-methylphenyl)picolinate (328 mg, 427.9 μmol, 45.2% yield, 98.5% purity) was obtained as a yellow oil.

MS (ESI) m/z: 755.5[M+H]⁺.

### Step C. Procedure for Preparation of 3-(4-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)phenyl)propanoic acid

A mixture of tert-butyl 6-(8-(benzo[d]thiazol-2-ylcarbamoyl)-3,4-dihydroisoquinolin-2(1H)-yl)-3-(4-(4-(3-methoxy-3-oxopropyl)phenoxy)-2-methylphenyl)picolinate (298 mg, 394.76 μmol, 1 equiv.), LiOH·H2O (82.83 mg, 1.97 mmol, 5 equiv.) and H₂O (1 mL) was added THF (3 mL). The mixture was stirred at 25° C. for 2 hours. The reaction mixture was concentrated under reduced pressure to give a residue. The residue was treated with water (4 mL). The pH was adjusted to around 3 by progressively adding diluted HCl. The mixture was filtered to give a residue. 3-(4-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)phenyl)propanoic acid (278 mg, 339.2 μmol, 85.9% yield, 90.4% purity) was obtained as a yellow solid.

MS (ESI) m/z: 741.4[M+H]⁺.

### Step D. Procedure for Preparation of tert-butyl 6-(8-(benzo[d]thiazol-2-ylcarbamoyl)-3,4-dihydroisoquinolin-2(1H)-yl)-3-(4-(4-(3-((3-(2,6-dioxopiperidin-3-yl)-1-methyl-1H-indazol-6-yl)amino)-3-oxopropyl)phenoxy)-2-methylphenyl)picolinate

A mixture of 3-(4-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)phenyl)propanoic acid (119 mg, 160.62 μmol, 1 equiv.), 3-(6-amino-1-methyl-1H-indazol-3-yl)piperidine-2,6-dione (49.78 mg, 192.75 μmol, 1.2 equiv.) in pyridine (2 mL) was added EDCI (46.19 mg, 240.93 μmol, 1.5 equiv.). The mixture was stirred at 25° C. for 16 hours. The reaction mixture was added into H₂O (5 mL), filtered, and concentrated under reduced pressure to give a residue. The crude product tert-butyl 6-(8-(benzo[d]thiazol-2-ylcarbamoyl)-3,4-dihydroisoquinolin-2(1H)-yl)-3-(4-(4-(3-((3-(2,6-dioxopiperidin-3-yl)-1-methyl-1H-indazol-6-yl)amino)-3-oxopropyl)phenoxy)-2-methylphenyl)picolinate (180 mg, crude) was obtained as a yellow oil and used into the next step without further purification.

MS (ESI) m/z: 982.5[M+H]⁺.

### Step E. Procedure for Preparation of 6-(8-(benzo[d]thiazol-2-ylcarbamoyl)-3,4-dihydroisoquinolin-2(1H)-yl)-3-(4-(4-(3-((3-(2,6-dioxopiperidin-3-yl)-1-methyl-1H-indazol-6-yl)amino)-3-oxopropyl)phenoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(4-(4-(3-((3-(2,6-dioxopiperidin-3-yl)-1-methyl-1H-indazol-6-yl)amino)-3-oxopropyl)phenoxy)-2-methylphenyl)picolinate (160 mg, 163.08 μmol, 1 equiv.) in CH₂Cl₂ (1.5 mL) was added TFA (2.05 g, 18.01 mmol, 1.33 mL, 110.43 equiv.). The mixture was stirred at 40° C. for 16 hours. The reaction mixture was concentrated under reduced pressure to give a residue. The residue was purified by prep-HPLC. 6-(8-(benzo[d]thiazol-2-ylcarbamoyl)-3,4-dihydroisoquinolin-2(1H)-yl)-3-(4-(4-(3-((3-(2,6-dioxopiperidin-3-yl)-1-methyl-1H-indazol-6-yl)amino)-3-oxopropyl)phenoxy)-2-methylphenyl)picolinic acid (76.1 mg, 80.0 μmol, 49.1% yield, 97.4% purity) was obtained as a yellow solid.

MS (ESI) m/z: 925.5[M+H]⁺.

¹HNMR (400 MHz, DMSO-d₆) δ=13.02-12.46 (m, 2H), 10.90-10.84 (m, 1H), 10.11 (s, 1H), 8.10-8.00 (m, 2H), 7.79 (d, J=8.0 Hz, 1H), 7.65-7.58 (m, 2H), 7.50-7.43 (m, 3H), 7.40-7.33 (m, 2H), 7.29 (d, J=8.4 Hz, 2H), 7.07 (dd, J=1.2, 8.8 Hz, 1H), 7.01-6.93 (m, 4H), 6.84 (d, J=2.4 Hz, 1H), 6.73 (dd, J=2.4, 8.4 Hz, 1H), 4.98 (s, 2H), 4.30 (dd, J=5.2, 9.6 Hz, 1H), 3.95-3.87 (m, 5H), 3.02 (t, J=5.6 Hz, 2H), 2.93 (t, J=7.6 Hz, 2H), 2.71-2.61 (m, 4H), 2.36-2.30 (m, 1H), 2.20-2.14 (m, 1H), 2.01 (s, 3H)

### Example 137. Preparation of Compound 147a

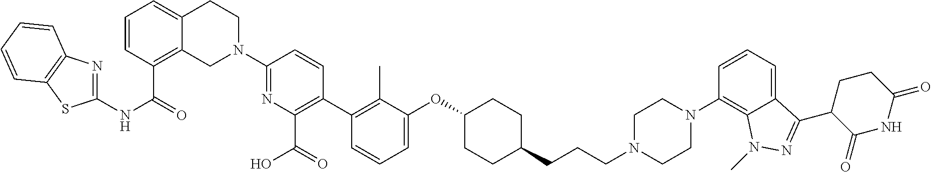 [SMILES: Cc1c(O[C@H]2CC[C@H](CCCN3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-7-yl)piperazin-1-yl)propyl)cyclohexyl)oxy)-2-methylphenyl)picolinic acid

### Step A. Procedure for Preparation of 3-((1r, 4s)-4-(3-bromo-2-methylphenoxy)cyclohexyl)propan-1-ol

To a solution of ethyl 3-((1r, 4r)-4-(3-bromo-2-methylphenoxy)cyclohexyl)propanoate (300 mg, 812.36 μmol, 1 equiv.) in THE (5 mL) was added LiAlH₄ (37.00 mg, 974.84 μmol, 1.2 equiv.) under 0° C. Then the mixture was stirred at 25° C. for 1.5 hours. The reaction mixture was quenched by addition H₂O 0.5 mL, 15% NaOH 0.5 mL, H₂O 1.5 mL, and then extracted with EtOAc 3 mL (1 mL×3). The combined organic layers were dried over Na₂SO₄, filtered and concentrated under reduced pressure to give 3-((1r, 4s)-4-(3-bromo-2-methylphenoxy)cyclohexyl)propan-1-ol (277 mg, crude) as a white solid.

### Step B. Procedure for Preparation of 3-((1r,4r)-4-(3-bromo-2-methylphenoxy)cyclohexyl)propanal

To a solution of DMSO (264.53 mg, 3.39 mmol, 264.53 μL, 4 equiv.) in DCM (2 mL) was added dropwise a solution of (COCl)₂ (214.88 mg, 1.69 mmol, 148.19 μL, 2 equiv.) in DCM (2 mL) at −70° C. under N₂ atmosphere. The mixture was stirred at −70° C. for 1 hour. After which time, 3-((1r, 4s)-4-(3-bromo-2-methylphenoxy)cyclohexyl)propan-1-ol (277 mg, 846.43 μmol, 1 equiv.) in DCM (2 mL) was added dropwise at −70° C. The solution was stirred for 1 hour at −70° C. Then TEA (513.90 mg, 5.08 mmol, 706.88 μL, 6 equiv.) was added into the solution. The solution was stirred at −70° C. for 0.5 hour under N₂ atmosphere. The reaction mixture was quenched by addition of H₂O (10 mL), and then diluted with DCM (10 mL) and extracted with DCM (10 mL×3). The combined organic layers were washed with H₂O (5 mL×3), dried over anhydrous sodium sulfate, filtered, and concentrated under reduced pressure to give a yellow residue. The residue was purified by flash silica gel chromatography (Eluent of 0˜10% ethyl acetate/petroleum ether) to give 3-((1r,4r)-4-(3-bromo-2-methylphenoxy)cyclohexyl)propanal (220 mg, 676.42 μmol, 79.91% yield) as a yellow solid.

### Step C. Procedure for Preparation of tert-butyl 6-(8-(benzo[d]thiazol-2-ylcarbamoyl)-3,4-dihydroisoquinolin-2(1H)-yl)-3-(2-methyl-3-(((1r,4r)-4-(3 oxopropyl)cyclohexyl)oxy)phenyl)picolinate

A mixture of 3-((1r,4r)-4-(3-bromo-2-methylphenoxy)cyclohexyl)propanal (220 mg, 676.42 μmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (497.21 mg, 811.71 μmol, 1.2 equiv.), [2-(2-aminophenyl)phenyl]palladium(1+); bis(1-adamantyl)-butyl-phosphane; methanesulfonate (98.52 mg, 135.28 μmol, 0.2 equiv.), and K₂CO₃ (1.5 M, 676.42 μL, 1.5 equiv.) in dioxane (5 mL) was degassed and purged with N₂ and stirred at 100° C. for 1 hour under microwave. The reaction mixture was filtered and concentrated under reduced pressure to give a residue. The residue was purified by flash silica gel chromatography (Eluent of 0˜10% ethyl acetate/petroleum ether) to give tert-butyl 6-(8-(benzo[d]thiazol-2-ylcarbamoyl)-3,4-dihydroisoquinolin-2(1H)-yl)-3-(2-methyl-3-(((1r,4r)-4-(3 oxopropyl)cyclohexyl)oxy)phenyl)picolinate (280 mg, 383.08 μmol, 56.63% yield) as a white solid.

MS (ESI) m/z: 731.5 [M+H]⁺.

¹H NMR (400 MHz, DMSO-d₆) δ=12.86-12.82 (m, 1H), 9.94 (s, 1H), 8.03 (d, J=7.6 Hz, 1H), 7.79 (d, J=8.0 Hz, 1H), 7.69-7.64 (m, 1H), 7.57 (d, J=8.0 Hz, 1H), 7.50-7.44 (m, 2H), 7.41-7.32 (m, 4H), 7.20 (d, J=7.2 Hz, 1H), 7.06 (d, J=8.4 Hz, 1H), 5.07-4.89 (m, 3H), 2.98 (t, J=6.0 Hz, 2H), 1.99 (s, 3H), 1.36 (s, 2H), 1.33 (s, 4H), 1.24 (s, 9H), 1.16 (s, 9H).

### Step D. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-7-yl)piperazin-1-yl)propyl)cyclohexyl)oxy)-2-methylphenyl)picolinate

A mixture of tert-butyl 6-(8-(benzo[d]thiazol-2-ylcarbamoyl)-3,4-dihydroisoquinolin-2(1H)-yl)-3-(2-methyl-3-(((1r,4r)-4-(3 oxopropyl)cyclohexyl)oxy)phenyl)picolinate (80 mg, 109.45 μmol, 1 equiv.), 3-(1-methyl-7-piperazin-1-yl-indazol-3-yl)piperidine-2,6-dione (43.00 mg, 131.34 μmol, 1.2 equiv.), and NaBH(OAc)₃ (69.59 mg, 328.36 μmol, 3 equiv.) in DCM (2 mL) was degassed and purged with N₂ three times, and then the mixture was stirred at 0° C. for 1.5 hour under N₂ atmosphere. The reaction mixture was filtered and concentrated under reduced pressure to give tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-7-yl)piperazin-1-yl)propyl)cyclohexyl)oxy)-2-methylphenyl)picolinate (100 mg, 95.9 μmol, 87.6% yield) as a white solid.

### Step E. Procedure for Preparation of 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-7-yl)piperazin-1-yl)propyl)cyclohexyl)oxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-7-yl)piperazin-1-yl)propyl)cyclohexyl)oxy)-2-methylphenyl)picolinate (80 mg, 76.75 μmol, 1 equiv.) in DCM (1 mL) and TFA (1 mL). The mixture was stirred at 40° C. for 3 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-7-yl)piperazin-1-yl)propyl)cyclohexyl)oxy)-2-methylphenyl)picolinic acid (45.5 mg, 44.3 μmol, 57.7% yield, 95.9% purity) as a white solid.

MS (ESI) m/z: 986.6 [M+H]⁺.

¹H NMR (400 MHz, DMSO-d₆) δ=12.91-12.83 (m, 1H), 10.89 (s, 1H), 8.14 (s, 1H), 8.04 (d, J=8.0 Hz, 1H), 7.79 (d, J=8.0 Hz, 1H), 7.63 (d, J=8.0 Hz, 1H), 7.51-7.34 (m, 7H), 7.08-7.03 (m, 3H), 6.95 (d, J=15.6 Hz, 2H), 6.62 (d, J=7.2 Hz, 1H), 4.98 (s, 2H), 4.34 (d, J=9.6 Hz, 1H), 4.25 (s, 3H), 3.93 (t, J=5.6 Hz, 2H), 3.03 (t, J=5.6 Hz, 4H), 2.95-2.81 (m, 3H), 2.70-2.61 (m, 4H), 2.36-2.32 (m, 3H), 2.20-2.16 (m, 1H), 2.10-2.08 (m, 2H), 1.88 (s, 3H), 1.81 (d, J=11.2 Hz, 2H), 1.53 (d, J=8.0 Hz, 2H), 1.39-1.24 (m, 6H), 1.17-1.06 (m, 3H).

### Example 138. Preparation of Compound 156

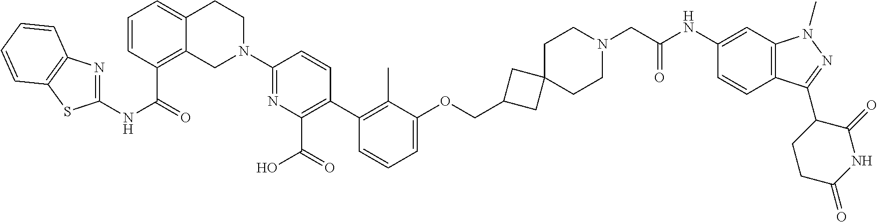 [SMILES: Cc1c(OCC2CC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-methylphenyl)picolinic acid

### Step A. Procedure for Preparation of tert-butyl 2-((3-bromo-2-methylphenoxy)methyl)-7-azaspiro[3.5]nonane-7-carboxylate

A mixture of tert-butyl 2-(hydroxymethyl)-7-azaspiro[3.5]nonane-7-carboxylate (1 g, 3.92 mmol, 1.2 equiv.), 3-bromo-2-methylphenol (610.38 mg, 3.26 mmol, 1 equiv.), and 2-(tributyl-λ⁵-phosphanylidene)acetonitrile (1.18 g, 4.90 mmol, 1.5 equiv.) in toluene (10 mL) was stirred at 120° C. for 2 h under N₂ atmosphere. The combined organic layers were filtered and concentrated under reduced pressure to give a residue. The residue was purified by flash silica gel chromatography (Eluent of 0˜8% ethyl acetate/petroleum ether) to give tert-butyl 2-((3-bromo-2-methylphenoxy)methyl)-7-azaspiro[3.5]nonane-7-carboxylate (1.34 g, 3.16 mmol, 96.76% yield) as a white solid.

¹H NMR (400 MHz, DMSO-d₆) δ=7.16 (d, J=8.0 Hz, 1H), 7.09 (t, J=8.0 Hz, 1H), 6.96 (d, J=8.0 Hz, 1H), 3.95 (d, J=6.2 Hz, 2H), 3.29-3.25 (m, 2H), 3.22-3.15 (m, 2H), 2.77-2.64 (m, 1H), 2.24 (s, 3H), 1.94-1.86 (m, 2H), 1.69-1.62 (m, 2H), 1.56-1.50 (m, 2H), 1.44-1.40 (m, 2H), 1.38 (s, 9H)

### Step B. Procedure for Preparation of 2-((3-bromo-2-methylphenoxy)methyl)-7-azaspiro[3.5]nonane

A mixture of tert-butyl 2-((3-bromo-2-methylphenoxy)methyl)-7-azaspiro[3.5]nonane-7-carboxylate (1.34 g, 3.16 mmol, 1 equiv.) in HCl/dioxane (15 mL) was stirred at 25° C. for 1 h. The combined organic layers were filtered and concentrated under reduced pressure to give a residue. The residue was used in the next step without other purification. The compound 2-((3-bromo-2-methylphenoxy)methyl)-7-azaspiro[3.5]nonane (1.32 g, crude) was obtained as a white solid.

¹H NMR (400 MHz, DMSO-d₆) δ=9.0-8.75 (m, 2H), 7.15 (d, J=7.2 Hz, 1H), 7.09 (t, J=8.0 Hz, 1H), 6.96 (d, J=8.0 Hz, 1H), 3.95 (d, J=6.2 Hz, 2H), 3.02-2.93 (m, 2H), 2.93-2.84 (m, 2H), 2.74-2.63 (m, 1H), 2.23 (s, 3H), 2.00-1.92 (m, 2H), 1.82-1.77 (m, 2H), 1.74-1.66 (m, 4H)

### Step C. Procedure for Preparation of ethyl 2-(2-((3-bromo-2-methylphenoxy)methyl)-7-azaspiro[3.5]nonan-7-yl)acetate

A mixture of 2-((3-bromo-2-methylphenoxy)methyl)-7-azaspiro[3.5]nonane (1.5 g, 4.63 mmol, 1.0 equiv.), ethyl 2-bromoacetate (772.54 mg, 4.63 mmol, 511.62 μL, 1.0 equiv.), KI (383.96 mg, 2.31 mmol, 0.5 equiv.), and K₂CO₃ (1.92 g, 13.88 mmol, 3.0 equiv.) in CH₃CN (15 mL) was stirred at 60° C. for 2 hours. The reaction mixture was filtered and concentrated under reduced pressure to give a residue. The mixture was purified by flash silica gel chromatography (Eluent of 0˜18% ethyl acetate/petroleum ether) to give ethyl 2-(2-((3-bromo-2-methylphenoxy)methyl)-7-azaspiro[3.5]nonan-7-yl)acetate (1.8 g, 4.4 mmol, 94.8% yield) as a yellow oil.

¹H NMR (400 MHz, DMSO-d₆) δ=7.14 (d, J=7.6 Hz, 1H), 7.07 (t, J=8.0 Hz, 1H), 6.94 (d, J=7.6 Hz, 1H), 4.06 (q, J=7.2 Hz, 2H), 3.92 (d, J=6.0 Hz, 2H), 3.14 (s, 2H), 2.70-2.61 (m, 1H), 2.45-2.40 (m, 2H), 2.36-2.31 (m, 2H), 2.23 (s, 3H), 1.89-1.83 (m, 2H), 1.64-1.57 (m, 4H), 1.50-1.47 (m, 2H), 1.18 (t, J=7.2 Hz, 3H)

### Step D. Procedure for Preparation of tert-butyl 6-(8-(benzo[d]thiazol-2-ylcarbamoyl)-3,4-dihydroisoquinolin-2(1H)-yl)-3-(3-((7-(2-ethoxy-2-oxoethyl)-7-azaspiro[3.5]nonan-2-yl)methoxy)-2-methylphenyl)picolinate

A mixture of ethyl 2-(2-((3-bromo-2-methylphenoxy)methyl)-7-azaspiro[3.5]nonan-7-yl)acetate (750 mg, 1.83 mmol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (1.34 g, 2.19 mmol, 1.2 equiv.), [2-(2-aminophenyl)phenyl]palladium(1⁺); bis(1-adamantyl)-butyl-phosphane; methanesulfonate (133.11 mg, 182.77 μmol, 0.1 equiv.), and K₂CO₃ (1.5 M, 1.83 mL, 1.5 equiv.) in dioxane (7.5 mL) was degassed and purged with N₂ three times, and then the mixture was stirred at 100° C. for 1 hour under microwave. The combined organic layers were filtered and concentrated under reduced pressure to give a residue. The mixture was purified by flash silica gel chromatography (Eluent of 48% ethyl acetate/petroleum ether) to give tert-butyl 6-(8-(benzo[d]thiazol-2-ylcarbamoyl)-3,4-dihydroisoquinolin-2(1H)-yl)-3-(3-((7-(2-ethoxy-2-oxoethyl)-7-azaspiro[3.5]nonan-2-yl)methoxy)-2-methylphenyl)picolinate (2.5 g, 3.0 mmol, 83.8% yield) as a yellow solid.

MS (ESI) m/z: 816.4[M+H]⁺.

### Step E. Procedure for Preparation of 2-(2-((3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)methyl)-7-azaspiro[3.5]nonan-7-yl)acetic acid

A mixture of tert-butyl 6-(8-(benzo[d]thiazol-2-ylcarbamoyl)-3,4-dihydroisoquinolin-2(1H)-yl)-3-(3-((7-(2-ethoxy-2-oxoethyl)-7-azaspiro[3.5]nonan-2-yl)methoxy)-2-methylphenyl)picolinate (1.9 g, 2.33 mmol, 1.0 equiv.) and LiOH·H₂O (488.50 mg, 11.64 mmol, 5 equiv.) in H₂O (7 mL) and THE (14 mL) was stirred at 25° C. for 16 hours. The reaction mixture was diluted with H₂O (5 mL) and concentrated as a turbid liquid. Then the pH of the turbid liquid was adjusted to 3 with 1 N HCl (5 mL), the residue was extracted with DCM/MeOH(20:1). The combined organic layers were filtered and concentrated to give 2-(2-((3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)methyl)-7-azaspiro[3.5]nonan-7-yl)acetic acid (2.1 g, crude) was obtained as a yellow solid.

MS (ESI) m/z: 788.3 [M+H]⁺.

### Step F. Procedure for Preparation of tert-butyl 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-methylphenyl)picolinate

To a solution of 2-(2-((3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)methyl)-7-azaspiro[3.5]nonan-7-yl)acetic acid (80 mg, 101.53 μmol, 1 equiv.) and 3-(6-amino-1-methyl-indazol-3-yl)piperidine-2,6-dione (31.47 mg, 121.83 umol, 1.2 equiv.) in pyridine (0.5 mL) was added EDCI (29.19 mg, 152.29 μmol, 1.5 equiv.). The mixture was stirred at 25° C. for 2 hours. The reaction mixture was quenched by the addition of water (1 mL). The organic phase was separated, extracted with ethyl acetate (1 mL×3), and concentrated under reduced pressure to give a residue to give tert-butyl 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-methylphenyl)picolinate (150 mg, crude) as a brown oil.

MS (ESI) m/z: 1028.7 [M+H]⁺

### Step G. Procedure for Preparation of 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-methylphenyl)picolinic acid

A solution of tert-butyl 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-methylphenyl)picolinate (80 mg, 77.80 umol, 1 equiv.) in DCM (1 mL) and TFA (1.54 g, 13.51 mmol, 1 mL, 173.59 equiv.) was stirred at 25° C. for 12 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-methylphenyl)picolinic acid (34.54 mg, 35.14 μmol, 45.16% yield, 98.9% purity) as a white solid.

MS (ESI) m/z: 972.7 [M+H]⁺.

¹HNMR (400 MHz, DMSO-d₆) δ=10.88 (s, 1H), 9.83 (s, 1H), 8.15 (s, 1H), 8.04-8.01 (m, 2H), 7.78 (d, J=8.0 Hz, 1H), 7.62 (d, J=8.4 Hz, 2H), 7.49-7.40 (m, 3H), 7.40-7.32 (m, 2H), 7.24-7.05 (m, 2H), 6.95 (d, J=8.8 Hz, 1H), 6.88 (d, J=8.4 Hz, 1H), 6.63 (d, J=7.6 Hz, 1H), 4.98 (s, 1H), 4.32 (dd, J=5.6, 9.6 Hz, 1H), 3.95-3.88 (m, 7H), 3.10 (s, 2H), 3.02 (t, J=5.5 Hz, 2H), 2.70-2.60 (m, 4H), 2.39-2.30 (m, 4H), 2.21-2.13 (m, 1H), 1.94-1.86 (m, 5H), 1.73-1.64 (m, 4H), 1.60 (s, 2H).

### Example 139. Preparation of Compound 157

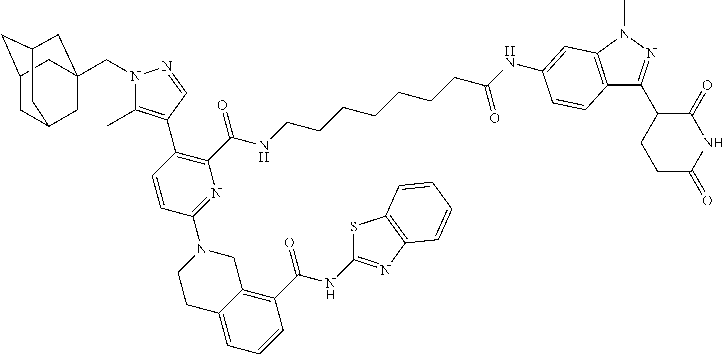 [SMILES: Cc1c(-c2ccc(N3CCc4cccc(C(=O)Nc5nc6ccccc6s5)c4C3)nc2C(=O)NCCCCCCCC(=O)Nc2ccc3c(C4CCC(=O)NC4=O)nn(C)c3c2)cnn1CC12CC3CC(CC(C3)C1)C2]

### 2-(5-(1-(((3r,5r,7r)-adamantan-1-yl)methyl)-5-methyl-1H-pyrazol-4-yl)-6-((8-((3-(2,6-dioxopiperidin-3-yl)-1-methyl-1H-indazol-6-yl)amino)-8-oxooctyl)carbamoyl)pyridin-2-yl)-N-(benzo[d]thiazol-2-yl)-1,2,3,4-tetrahydroisoquinoline-8-carboxamide

### Step A. Procedure for Preparation of 2-(5-(1-(((3r,5r,7r)-adamantan-1-yl)methyl)-5-methyl-1H-pyrazol-4-yl)-6-((8-((3-(2,6-dioxopiperidin-3-yl)-1-methyl-1H-indazol-6-yl)amino)-8-oxooctyl)carbamoyl)pyridin-2-yl)-N-(benzo[d]thiazol-2-yl)-1,2,3,4-tetrahydroisoquinoline-8-carboxamide

A mixture of 8-[[3-[1-(1-adamantylmethyl)-5-methyl-pyrazol-4-yl]-6-[8-(1,3-benzothiazol-2-ylcarbamoyl)-3,4-dihydro-1H-isoquinolin-2-yl]pyridine-2-carbonyl]amino]octanoic acid (110 mg, 137.50 μmol, 1 equiv.), 3-(6-amino-1-methyl-indazol-3-yl)piperidine-2,6-dione (42.61 mg, 165.00 μmol, 1.2 equiv.), and HATU (62.74 mg, 165.00 μmol, 1.2 equiv.) in DMF (1.4 mL) was degassed and purged with N₂ and stirred at 25° C. for 5 minutes. After 5 minutes, to the mixture was added DIPEA (53.31 mg, 412.49 μmol, 71.85 μL, 3 equiv.). The mixture was stirred at 25° C. for 1 hour under N₂ atmosphere. The reaction mixture was diluted with water (20 mL) and extracted with DCM (25 mL×3). The combined organic layers were washed with brine (15 mL×3), dried over anhydrous sodium sulfate filtered, and concentrated under reduced pressure to give a residue. The compound 2-(5-(1-(((3r,5r,7r)-adamantan-1-yl)methyl)-5-methyl-1H-pyrazol-4-yl)-6-((8-((3-(2,6-dioxopiperidin-3-yl)-1-methyl-1H-indazol-6-yl)amino)-8-oxooctyl)carbamoyl)pyridin-2-yl)-N-(benzo[d]thiazol-2-yl)-1,2,3,4-tetrahydroisoquinoline-8-carboxamide (139.8 mg, 128.6 μmol, 93.5% yield, 95.7% purity) was obtained as a yellow solid.

MS (ESI) m/z: 520.9 [M+H]⁺

¹H NMR (400 MHz, CD₃OD) δ=8.33 (t, J=6.4 Hz, 1H), 8.05 (s, 1H), 7.91 (d, J=8.0 Hz, 1H), 7.79-7.76 (m, 1H), 7.60 (d, J=8.8 Hz, 1H), 7.60-7.53 (m, 1H), 7.47-7.43 (m, 2H), 7.33 (t, J=4.0 Hz, 3H), 7.05 (dd, J=8.4, 1.2 Hz, 1H), 6.99 (d, J=8.8 Hz, 1H), 5.09 (s, 2H), 4.33 (q, J=5.2 Hz, 1H), 3.93 (s, 3H), 3.90 (t, J=6.4 Hz, 2H), 3.72 (s, 2H), 3.21-3.11 (m, 3H), 3.04 (t, J=6.0 Hz, 2H), 2.81-2.70 (m, 2H), 2.37 (t, J=7.6 Hz, 2H), 2.09 (s, 3H), 1.98-1.92 (m, 4H), 1.74-1.69 (m, 4H), 1.65 (s, 4H), 1.62-1.61 (m, 2H), 1.60-1.57 (m, 6H), 1.47-1.43 (m, 2H), 1.31-1.28 (m, 4H)

### Example 140. Preparation of Compound 158

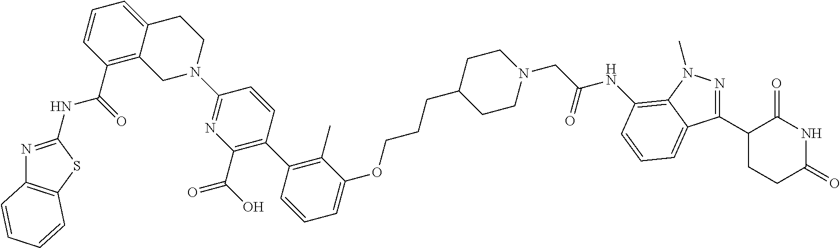 [SMILES: Cc1c(OCCCC2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-7-yl)amino)-2-oxoethyl)piperidin-4-yl)propoxy)-2-methylphenyl)picolinic acid

### Step A. Procedure for Preparation of tert-butyl 4-(3-(3-bromo-2-methylphenoxy)propyl)piperidine-1-carboxylate

A mixture of 3-bromo-2-methyl-phenol (2 g, 10.69 mmol, 1 equiv.), K₂CO₃ (4.43 g, 32.08 mmol, 3 equiv.), and tert-butyl 4-(3-hydroxypropyl)piperidine-1-carboxylate (3.27 g, 10.69 mmol, 1 equiv.) in CH₃CN (20 mL) was degassed and purged with N₂ and stirred at 60° C. for 12 hours under N₂ atmosphere. The reaction mixture was concentrated under reduced pressure to give a residue. The residue was purified by flash silica gel chromatography (Eluent of 0˜7% ethyl acetate/petroleum ether) to give tert-butyl 4-(3-(3-bromo-2-methylphenoxy)propyl)piperidine-1-carboxylate (1.8 g, 4.3 mmol, 40.8% yield) as a colorless oil

¹H NMR (400 MHz, CDCl₃) δ=7.09-7.07 (m, 1H), 6.91 (t, J=8.0 Hz, 1H), 6.68 (s, 1H), 3.86 (t, J=6.4 Hz, 2H), 2.63 (t, J=12.0 Hz, 2H), 2.23 (s, 3H), 1.79-1.69 (m, 2H), 1.69-1.50 (m, 4H), 1.39 (s, 9H), 1.38-1.30 (m, 3H), 1.11-0.98 (m, 2H)

### Step B. Procedure for Preparation of 4-(3-(3-bromo-2-methylphenoxy)propyl)piperidine

A mixture of tert-butyl 4-(3-(3-bromo-2-methylphenoxy)propyl)piperidine-1-carboxylate (1.8 g, 4.37 mmol, 1 equiv.) in HCl/EtOAc (20 mL) was stirred at 20° C. for 1 hour. The reaction mixture was concentrated under reduced pressure to give 4-(3-(3-bromo-2-methylphenoxy)propyl)piperidine (1.5 g, crude) as a white solid.

MS (ESI) m/z: 313.9 [M+3]⁺.

### Step C. Procedure for Preparation of ethyl 2-(4-(3-(3-bromo-2-methylphenoxy)propyl)piperidin-1-yl)acetate

A mixture of 4-(3-(3-bromo-2-methylphenoxy)propyl)piperidine (1 g, 3.20 mmol, 1 equiv.), ethyl 2-bromoacetate (534.84 mg, 3.20 mmol, 354.20 μL, 1 equiv.) and K₂CO₃ (885.24 mg, 6.41 mmol, 2 equiv.) in CH₃CN (15 mL) was degassed and purged with N₂ and stirred at 60° C. for 2 hours under N₂ atmosphere. The reaction mixture was concentrated under reduced pressure to give a residue. The residue was purified by flash silica gel chromatography (Eluent of 0˜45% ethyl acetate/petroleum ether) to give ethyl 2-(4-(3-(3-bromo-2-methylphenoxy)propyl)piperidin-1-yl)acetate (0.5 g, 1.2 mmol, 39.1% yield) as a yellow oil MS (ESI) m/z: 399.9 [M+3]⁺.

¹H NMR (400 MHz, CD₃OD) δ=7.14 (d, J=8.0 Hz, 1H), 6.98 (t, J=8.0 Hz, 1H), 6.75 (d, J=8.0 Hz, 1H), 4.19 (q, J=7.2 Hz, 2H), 3.93 (t, J=6.4 Hz, 2H), 3.23-3.18 (m, 2H), 2.95 (d, J=11.6 Hz, 2H), 2.31 (s, 3H), 2.18-2.11 (m, 2H), 1.84-1.79 (m, 2H), 1.72-1.64 (m, 4H), 1.59-1.53 (m, 2H), 1.46-1.40 (m, 4H), 1.35 (dd, J=3.2, 10.8 Hz, 2H), 1.28 (t, J=7.2 Hz, 3H)

### Step D. Procedure for Preparation of tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)piperidin-4-yl)propoxy)-2-methylphenyl)picolinate

A mixture of ethyl 2-(4-(3-(3-bromo-2-methylphenoxy)propyl)piperidin-1-yl)acetate (500 mg, 1.26 mmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (768.89 mg, 1.26 mmol, 1 equiv.), [2-(2-aminophenyl)phenyl]palladium(1⁺); bis(1-adamantyl)-butyl-phosphane; methanesulfonate (91.41 mg, 125.52 μmol, 0.1 equiv.) and K₃PO₄ (1.5 M, 2.51 mL, 3 equiv.) in 1,4-dioxane (5 mL) was degassed and purged with N₂ and stirred at 80° C. for 12 hours under N₂ atmosphere. The reaction mixture was concentrated under reduced pressure to give a residue. The residue was purified by flash silica gel chromatography (Eluent of 0˜5% ethyl acetate/petroleum ether) to give tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)piperidin-4-yl)propoxy)-2-methylphenyl)picolinate (800 mg, 696.5 μmol, 55.4% yield, 70% purity) as a yellow solid.

MS (ESI) m/z: 804.5 [M+H]⁺.

### Step E. Procedure for Preparation of 2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)acetic acid

To a solution of tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)piperidin-4-yl)propoxy)-2-methylphenyl)picolinate (342.86 mg, 298.51 μmol, 70% purity, 1 equiv.) in THF (3 mL) was added LiOH (1 M, 895.52 μL, 3 equiv.). The mixture was stirred at 20° C. for 1 hour. The reaction mixture was adjusted to pH=5, filtered, and concentrated under reduced pressure to give 2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)acetic acid (150 mg, 193.3 μmol, 64.7% yield) as a yellow solid.

MS (ESI) m/z: 776.6 [M+H]⁺

### Step F. Procedure for Preparation of tert-butyl 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-7-yl)amino)-2-oxoethyl)piperidin-4-yl)propoxy)-2-methylphenyl)picolinate

A mixture of 2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)acetic acid (70 mg, 90.21 μmol, 1 equiv.), 3-(7-amino-1-methyl-1H-indazol-3-yl)piperidine-2,6-dione (23.30 mg, 90.21 μmol, 1 equiv.), HATU (37.73 mg, 99.23 μmol, 1.1 equiv.), and TEA (27.39 mg, 270.64 μmol, 37.67 μL, 3 equiv.) in DMF (1 mL) was degassed and purged with N₂ and stirred at 40° C. for 12 hours under N₂ atmosphere. The solution was poured into water (2 mL) and filtered to give tert-butyl 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-7-yl)amino)-2-oxoethyl)piperidin-4-yl)propoxy)-2-methylphenyl)picolinate (60 mg, 59.0 μmol, 65.4% yield) as a yellow solid.

MA (ESI) m/z: 508.9 [M/2+H]⁺

### Step G. Procedure for Preparation of 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-7-yl)amino)-2-oxoethyl)piperidin-4-yl)propoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-7-yl)amino)-2-oxoethyl)piperidin-4-yl)propoxy)-2-methylphenyl)picolinate (60 mg, 59.04 μmol, 1 equiv.) in DCM (0.5 mL) was added TFA (0.5 mL). The mixture was stirred at 40° C. for 0.5 hour. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-7-yl)amino)-2-oxoethyl)piperidin-4-yl)propoxy)-2-methylphenyl)picolinic acid (29.1 mg, 29.4 μmol, 49.8% yield, 96.8% purity) as a yellow solid

MS (ESI) m/z: 960.6 [M+1]⁺.

¹H NMR (400 MHz, DMSO-d₆) δ=13.15-12.41 (m, 1H), 10.94-10.81 (m, 1H), 9.92-9.73 (m, 1H), 8.03 (d, J=7.2 Hz, 1H), 7.79 (d, J=8.0 Hz, 1H), 7.63 (d, J=6.4 Hz, 1H), 7.60-7.54 (m, 1H), 7.52-7.20 (m, 7H), 7.13-7.05 (m, 2H), 7.01-6.93 (m, 1H), 6.88 (d, J=8.0 Hz, 1H), 6.64 (d, J=7.2 Hz, 1H), 4.99 (s, 2H), 4.38 (dd, J=4.8, 9.2 Hz, 1H), 4.12 (s, 3H), 4.03-3.85 (m, 4H), 3.03 (s, 4H), 2.98-2.94 (m, 2H), 2.69-2.62 (m, 2H), 2.22-2.17 (m, 2H), 1.90 (s, 3H), 1.83-1.67 (m, 5H), 1.46-1.26 (m, 6H)

### Example 141. Preparation of Compound 159a

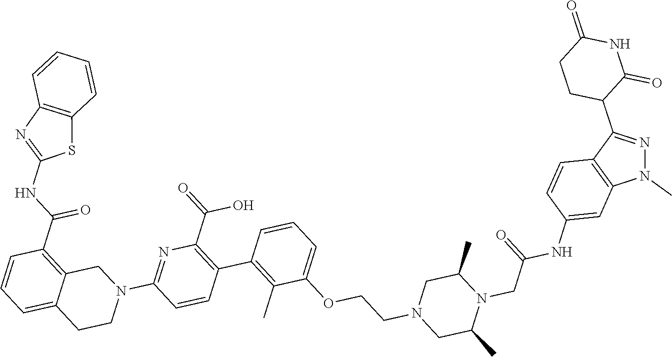 [SMILES: Cc1c(OCCN2C[C@@H](C)N(CC(=O)Nc3ccc4c(C5CCC(=O)NC5=O)nn(C)c4c3)[C@@H](C)C2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(2-((3R,5S)-4-(2-((3-(2,6-dioxopiperidin-3-yl)-1-methyl-1H-indazol-6-yl)amino)-2-oxoethyl)-3,5-dimethylpiperazin-1-yl)ethoxy)-2-methylphenyl)picolinic acid

### Step A. Procedure for Preparation of ethyl 2-[(2R, 6S)-4-[2-(3-bromo-2-methyl-phenoxy)ethyl]-2,6-dimethyl-piperazin-1-yl]acetate

A mixture of (3R,5S)-1-[2-(3-bromo-2-methyl-phenoxy)ethyl]-3,5-dimethyl-piperazine (2.7 g, 8.25 mmol, 1 equiv.), ethyl 2-bromoacetate (3.44 g, 20.63 mmol, 2.28 mL, 2.5 equiv.), and Et₃N (3.34 g, 33.00 mmol, 4.59 mL, 4 equiv.) in DMF (10 mL) was degassed and purged with N₂ and stirred at 50° C. for 4 hours under N₂ atmosphere. The reaction mixture was filtered and concentrated under reduced pressure to give a residue. The residue was purified by column chromatography (SiO₂, dichloromethane:methanol=I/O to 5/1). The compound ethyl 2-[(2R, 6S)-4-[2-(3-bromo-2-methyl-phenoxy)ethyl]-2,6-dimethyl-piperazin-1-yl]acetate (1.2 g, 2.9 mmol, 35.1% yield) was obtained as a white solid.

MS (ESI) m/z: 414.9 [M+H]⁺.

¹H NMR (400 MHz, CDCl₃) δ=7.20 (d, J=8.0 Hz, 1H), 7.02 (t, J=8.0 Hz, 1H), 6.79 (d, J=8.0 Hz, 1H), 4.38 (s, 2H), 4.20 (q, J=6.8 Hz, 2H), 3.56 (s, 2H), 3.42-3.40 (m, 2H), 3.24-3.18 (m, 4H), 2.53-2.46 (m, 2H), 2.30 (s, 3H), 1.31 (t, J=7.2 Hz, 3H), 1.14 (d, J=6.0 Hz, 6H).

### Step B. Procedure for Preparation of tert-butyl 6-[8-(1,3-benzothiazol-2-ylcarbamoyl)-3,4-dihydro-1H-isoquinolin-2-yl]-3-[3-[2-[(3S,5R)-4-(2-ethoxy-2-oxo-ethyl)-3,5-dimethyl-piperazin-1-yl]ethoxy]-2-methyl-phenyl]pyridine-2-carboxylate

To a solution of ethyl 2-[(2R, 6S)-4-[2-(3-bromo-2-methyl-phenoxy)ethyl]-2,6-dimethyl-piperazin-1-yl]acetate (1.2 g, 2.90 mmol, 1 equiv.) and tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (1.96 g, 3.19 mmol, 1.1 equiv.) in 1,4-dioxane (8 mL) was added KF (1.5 M, 5.81 mL, 3 equiv.) and ditert-butyl(cyclopentyl)phosphane; dichloropalladium; iron (189.21 mg, 290.31 μmol, 0.1 equiv.). The mixture was stirred at 100° C. for 1 hour under microwave. The reaction mixture was filtered and concentrated under reduced pressure to give a residue. The residue was purified by column chromatography (SiO₂, petroleum ether/ethyl acetate=1/1). The compound tert-butyl 6-[8-(1,3-benzothiazol-2-ylcarbamoyl)-3,4-dihydro-1H-isoquinolin-2-yl]-3-[3-[2-[(3S,5R)-4-(2-ethoxy-2-oxo-ethyl)-3,5-dimethyl-piperazin-1-yl]ethoxy]-2-methyl-phenyl] pyridine-2-carboxylate (2 g, 2.4 mmol, 84.1% yield) was obtained as a yellow solid.

MS (ESI) m/z: 819.3 [M+H]⁺.

### Step C. Procedure for Preparation of 2-((2R,6S)-4-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethyl)-2,6-dimethylpiperazin-1-yl)acetic acid

To a solution of tert-butyl 6-[8-(1,3-benzothiazol-2-ylcarbamoyl)-3,4-dihydro-1H-isoquinolin-2-yl]-3-[3-[2-[(3S,5R)-4-(2-ethoxy-2-oxo-ethyl)-3,5-dimethyl-piperazin-1-yl]ethoxy]-2-methyl-phenyl]pyridine-2-carboxylate (2 g, 2.44 mmol, 1 equiv.) in THE (10 mL) was added LiOH·H₂O (1 M, 7.33 mL, 3 equiv.). The mixture was stirred at 25° C. for 2 hours. The reaction mixture was concentrated under reduced pressure to remove THF, and then acidified with HCl (1M), filtered, and concentrated under reduced pressure to give a residue. The compound 2-[(2S,6R)-4-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]ethyl]-2,6-dimethyl-piperazin-1-yl]acetic acid (1.9 g, 2.4 mmol, 98.3% yield) was obtained as a brown solid.

MS (ESI) m/z: 791.5 [M+H]⁺

### Step D. Procedure for Preparation of tert-butyl 6-(8-(benzo[d]thiazol-2-ylcarbamoyl)-3,4-dihydroisoquinolin-2(1H)-yl)-3-(3-(2-((3R,5S)-4-(2-((3-(2,6-dioxopiperidin-3-yl)-1-methyl-1H-indazol-6-yl)amino)-2-oxoethyl)-3,5-dimethylpiperazin-1-yl)ethoxy)-2-methylphenyl)picolinate

A mixture of 2-((2R,6S)-4-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethyl)-2,6-dimethylpiperazin-1-yl)acetic acid (160 mg, 202.28 μmol, 1.00 equiv.), 3-(6-amino-1-methyl-1H-indazol-3-yl)piperidine-2,6-dione (62.69 mg, 242.74 μmol, 1.20 equiv.), and DIPEA (78.43 mg, 606.85 μmol, 105.70 μL, 3.00 equiv.) in DMF (2 mL) was degassed and purged with N₂ three times, the mixture was stirred at 25° C. for 5 mins. After 5 mins, to the mixture was added HATU (92.30 mg, 242.74 μmol, 1.20 equiv.), and the mixture was stirred at 25° C. for 16 hours under N₂ atmosphere. The reaction mixture was diluted with water (30 mL) and extracted with DCM (30 mL×3). The combined organic layers were dried over anhydrous sodium sulfate filtered and concentrated under reduced pressure to give a crude tert-butyl 6-(8-(benzo[d]thiazol-2-ylcarbamoyl)-3,4-dihydroisoquinolin-2(1H)-yl)-3-(3-(2-((3R,5S)-4-(2-((3-(2,6-dioxopiperidin-3-yl)-1-methyl-1H-indazol-6-yl)amino)-2-oxoethyl)-3,5-dimethylpiperazin-1-yl)ethoxy)-2-methylphenyl)picolinate (280 mg, crude) as a brown solid.

MS (ESI) m/z: 1031.6[M+H]⁺

### Step E. Procedure for Preparation of 6-(8-(benzo[d]thiazol-2-ylcarbamoyl)-3,4-dihydroisoquinolin-2(1H)-yl)-3-(3-(2-((3R,5S)-4-(2-((3-(2,6-dioxopiperidin-3-yl)-1-methyl-1H-indazol-6-yl)amino)-2-oxoethyl)-3,5-dimethylpiperazin-1-yl)ethoxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(3-(2-((3R,5S)-4-(2-((3-(2,6-dioxopiperidin-3-yl)-1-methyl-1H-indazol-6-yl)amino)-2-oxoethyl)-3,5-dimethylpiperazin-1-yl)ethoxy)-2-methylphenyl)picolinate (280 mg, 271.52 μmol, 1.00 equiv.) in DCM (1.4 mL) and TFA (1.4 mL) was degassed and purged with N₂ three times, and then the mixture was stirred at 25° C. for 16 hours under N₂ atmosphere. The reaction mixture was concentrated under reduced pressure to give a residue. The residue was purified by reverse-phase HPLC to give 6-(8-(benzo[d]thiazol-2-ylcarbamoyl)-3,4-dihydroisoquinolin-2(1H)-yl)-3-(3-(2-((3R,5S)-4-(2-((3-(2,6-dioxopiperidin-3-yl)-1-methyl-1H-indazol-6-yl)amino)-2-oxoethyl)-3,5-dimethylpiperazin-1-yl)ethoxy)-2-methylphenyl)picolinic acid (81.8 mg, 78.0 μmol, 28.7% yield, 92.4% purity) as a pink solid.

MS (ESI) m/z: 957.7 [M+H]⁺

¹H NMR (400 MHz, DMSO-d₆) δ=13.02-12.70 (m, 1H), 10.88 (s, 1H), 9.80 (s, 1H), 8.17-8.11 (m, 1H), 8.02 (s, 2H), 7.78 (d, J=8.0 Hz, 1H), 7.68-7.58 (m, 2H), 7.50-7.30 (m, 6H), 7.22 (d, J=7.2 Hz, 1H), 7.16-7.08 (m, 1H), 7.01-6.89 (m, 2H), 6.66 (d, J=6.8 Hz, 1H), 4.98 (s, 2H), 4.37-4.29 (m, 1H), 4.23-4.06 (m, 2H), 3.92 (s, 4H), 3.21-2.77 (m, 9H), 2.74-2.58 (m, 4H), 2.44-2.27 (m, 2H), 2.26-2.07 (m, 2H), 1.91 (s, 3H), 1.02 (s, 6H)

### Example 142. Preparation of Compound 166

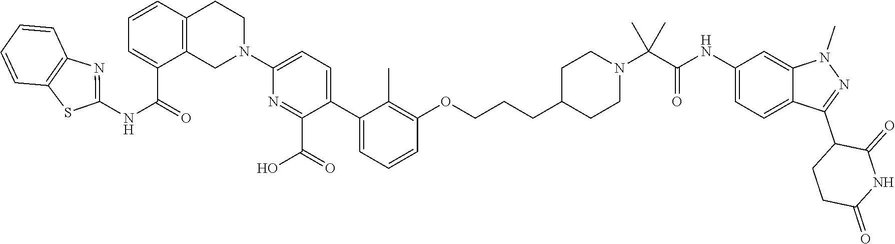 [SMILES: Cc1c(OCCCC2CCN(C(C)(C)C(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-1,1-dimethyl-2-oxo-ethyl]-4-piperidyl]propoxy]-2-methyl-phenyl]pyridine-2-carboxylic acid

### Step A. Procedure for Preparation of methyl 2-[4-[3-(3-bromo-2-methyl-phenoxy)propyl]-1-piperidyl]-2-methyl-propanoate

To a solution of 4-[3-(3-bromo-2-methyl-phenoxy)propyl]piperidine (500 mg, 1.60 mmol, 1 equiv.) and methyl 2-bromo-2-methyl-propanoate (347.86 mg, 1.92 mmol, 248.47 μL, 1.2 equiv.) in CH₃CN (2 mL) was added K₂CO₃ (663.95 mg, 4.80 mmol, 3 equiv.) and KI (132.91 mg, 800.65 μmol, 0.5 equiv.). The mixture was stirred at 80° C. for 12 hours. The reaction mixture was filtered and concentrated under reduced pressure to give a residue. The residue was purified by column chromatography (SiO₂, petroleum ether/ethyl acetate=0/1 to 1/1) to give methyl 2-[4-[3-(3-bromo-2-methyl-phenoxy)propyl]-1-piperidyl]-2-methyl-propanoate (340 mg, 824.5 μmol, 51.4% yield) as a yellow oil.

MS (ESI) m/z: 412.2 [M+H]⁺

### Step B. Procedure for Preparation of tert-butyl 6-[8-(1,3-benzothiazol-2-ylcarbamoyl)-3,4-dihydro-1H-isoquinolin-2-yl]-3-[3-[3-[1-(2-methoxy-1,1-dimethyl-2-oxo-ethyl)-4-piperidyl]propoxy]-2-methyl-phenyl]pyridine-2-carboxylate

A mixture of methyl 2-[4-[3-(3-bromo-2-methyl-phenoxy)propyl]-1-piperidyl]-2-methyl-propanoate (340 mg, 824.52 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (505.06 mg, 824.52 μmol, 1 equiv.), Ad₂nBuP Pd G₃ (cataCXium® A Pd G₃) (60.05 mg, 82.45 μmol, 0.1 equiv.), and KF (1.5 M, 549.68 uL, 1 equiv.) in dioxane (2 mL) was degassed and purged with N₂ and stirred at 100° C. for 1 hour under N₂ atmosphere. The reaction mixture was filtered and concentrated under reduced pressure to give a residue. The residue was purified by column chromatography (SiO₂, petroleum ether/ethyl acetate=I/O to 1/1) to give tert-butyl 6-[8-(1,3-benzothiazol-2-ylcarbamoyl)-3,4-dihydro-1H-isoquinolin-2-yl]-3-[3-[3-[1-(2-methoxy-1,1-dimethyl-2-oxo-ethyl)-4-piperidyl]propoxy]-2-methyl-phenyl]pyridine-2-carboxylate (370 mg, 452.3 μmol, 54.8% yield) as a yellow oil.

MS (ESI) m/z: 818.7 [M+H]⁺

¹HNMR (400 MHz, CDCl₃) δ=11.11-10.17 (m, 1H), 7.87-7.82 (m, 1H), 7.56 (d, J=7.6 Hz, 1H), 7.48 (d, J=7.2 Hz, 1H), 7.38 (d, J=8.4 Hz, 1H), 7.35-7.31 (m, 2H), 7.31-7.27 (m, 1H), 7.25-7.22 (m, 1H), 7.09 (t, J=8.0 Hz, 1H), 6.90 (d, J=8.8 Hz, 1H), 6.80 (d, J=8.4 Hz, 1H), 6.68 (d, J=7.2 Hz, 1H), 5.12-4.95 (m, 2H), 4.11-4.08 (m, 2H), 3.96 (t, J=6.4 Hz, 2H), 3.71 (s, 3H), 3.06 (t, J=6.0 Hz, 2H), 2.91 (d, J=10.0 Hz, 2H), 2.16-2.08 (m, 2H), 1.98 (s, 3H), 1.85-1.77 (m, 2H), 1.73 (d, J=8.0 Hz, 2H), 1.45-1.38 (m, 2H), 1.35-1.28 (m, 9H), 1.15 (s, 9H).

### Step C. Procedure for Preparation of 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-1-piperidyl]-2-methyl-propanoic acid

To a solution of tert-butyl 6-[8-(1,3-benzothiazol-2-ylcarbamoyl)-3,4-dihydro-1H-isoquinolin-2-yl]-3-[3-[3-[1-(2-methoxy-1,1-dimethyl-2-oxo-ethyl)-4-piperidyl]propoxy]-2-methyl-phenyl]pyridine-2-carboxylate (370 mg, 452.30 μmol, 1 equiv.) in H₂O (1 mL), MeOH (1 mL) and THE (1 mL) was added LiOH·H₂O (189.80 mg, 4.52 mmol, 10 equiv.). The mixture was stirred at 50° C. for 2 hours. The pH was adjusted to 6 with 1M HCl. The mixture was extracted with DCM (2 mL×3). The combined organic layers were filtered and concentrated under reduced pressure to give a residue. The residue was purified by prep-TLC (SiO₂, DCM:MeOH=10:1) to give compound 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-1-piperidyl]-2-methyl-propanoic acid (166 mg, 206.4 μmol, 45.6% yield) as a yellow oil.

MS (ESI) m/z: 804.7 [M+H]⁺

### Step D. Procedure for Preparation of tert-butyl 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-1,1-dimethyl-2-oxo-ethyl]-4-piperidyl]propoxy]-2-methyl-phenyl]pyridine-2-carboxylate

To a solution of 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-1-piperidyl]-2-methyl-propanoic acid (100 mg, 124.38 μmol, 1 equiv.), 3-(6-amino-1-methyl-indazol-3-yl)piperidine-2,6-dione (48.19 mg, 186.57 μmol, 1.5 equiv.) in DMF (1 mL) was added HATU (118.23 mg, 310.94 μmol, 2.5 equiv.) and DIEA (48.22 mg, 373.13 μmol, 64.99 μL, 3 equiv.). The mixture was stirred at 40° C. for 4 hours. The reaction mixture was quenched by water (2 mL) at 25° C. filtered and concentrated under reduced pressure to give a residue. The residue was purified by prep-TLC (SiO₂, ethyl acetate) to give tert-butyl 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-1,1-dimethyl-2-oxo-ethyl]-4-piperidyl]propoxy]-2-methyl-phenyl]pyridine-2-carboxylate (50 mg, 47.8 μmol, 38.5% yield) as a colorless oil.

MS (ESI) m/z: 1044.9 [M+H]⁺

### Step E. Procedure for Preparation of 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-1,1-dimethyl-2-oxo-ethyl]-4-piperidyl]propoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-1,1-dimethyl-2-oxo-ethyl]-4-piperidyl]propoxy]-2-methyl-phenyl]pyridine-2-carboxylate (50 mg, 47.88 μmol, 1 equiv.) in DCM (1 mL) was added TFA (1 mL). The mixture was stirred at 25° C. for 12 hours. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-1,1-dimethyl-2-oxo-ethyl]-4-piperidyl]propoxy]-2-methyl-phenyl]pyridine-2-carboxylic acid (27.5 mg, 27.8 μmol, 58.1% yield, 99.9% purity) as a white solid.

MS (ESI) m/z: 988.8[M+H]⁺

¹HNMR (400 MHz, DMSO-d₆,) 6=13.49-11.83 (m, 1H), 10.88 (s, 1H), 9.74 (s, 1H), 8.16 (s, 1H), 8.08-7.99 (m, 2H), 7.78 (d, J=8.0 Hz, 1H), 7.62 (d, J=8.4 Hz, 2H), 7.49-7.41 (m, 3H), 7.39-7.32 (m, 2H), 7.23 (d, J=8.8 Hz, 1H), 7.11-7.06 (m, 1H), 6.95 (d, J=8.8 Hz, 1H), 6.87 (d, J=8.0 Hz, 1H), 6.63 (d, J=7.6 Hz, 1H), 4.98 (s, 2H), 4.32 (dd, J=5.2, 9.6 Hz, 1H), 3.95 (t, J=6.4 Hz, 2H), 3.92 (s, 5H), 3.18-3.17 (m, 1H), 3.17 (s, 2H), 3.02 (t, J=5.6 Hz, 2H), 2.74 (d, J=10.0 Hz, 2H), 2.67-2.59 (m, 2H), 2.15 (d, J=5.6 Hz, 2H), 1.90 (s, 3H), 1.81-1.70 (m, 4H), 1.45-1.39 (m, 2H), 1.32 (s, 3H), 1.19 (s, 6H).

### Example 143. Preparation of Compound 167a

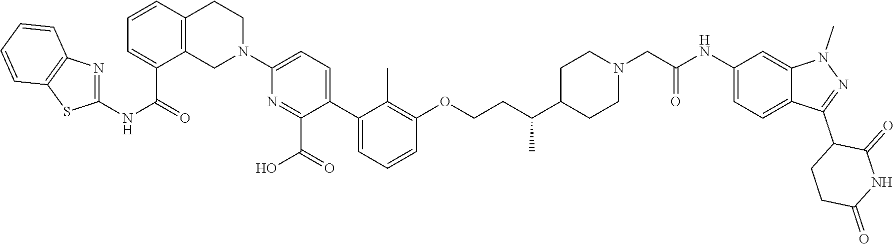 [SMILES: Cc1c(OCC[C@@H](C)C2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[(3R)-3-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylic acid

### Step A. Procedure for Preparation of tert-butyl 4-[(E)-3-ethoxy-1-methyl-3-oxo-prop-1-enyl]piperidine-1-carboxylate

To a solution of NaH (2.51 g, 62.69 mmol, 60% purity, 1.5 equiv.) in THE (100 mL) was added ethyl 2-diethoxyphosphorylacetate (11.24 g, 50.15 mmol, 9.95 mL, 1.2 equiv.) at 0° C. slowly. The reaction mixture was stirred at 0° C. for 1 hour, after which tert-butyl 4-acetylpiperidine-1-carboxylate (9.5 g, 41.80 mmol, 1 equiv.) was added at 0° C. slowly. The mixture was warmed to 25° C. and then stirred at 25° C. for 12 hours. The reaction mixture was cooled to 0° C., and saturated NH₄Cl solution was added dropwise. Then the mixture was diluted with H₂O (100 mL) and extracted with EtOAc (200 mL×3). The combined organic layers were washed with brine (300 mL×1), dried over Na₂SO₄, filtered, and concentrated under reduced pressure to give a residue. The residue was purified by column chromatography (SiO₂, petroleum ether/ethyl acetate=20/1 to 10/1) to give tert-butyl 4-[(E)-3-ethoxy-1-methyl-3-oxo-prop-1-enyl]piperidine-1-carboxylate (5.7 g, 15.4 mmol, 36.9% yield, 80.6% purity) as a yellow oil.

MS (ESI) m/z: 198.0 [M−100+H]⁺

¹H NMR (400 MHz, DMSO-d₆) δ=5.68-5.60 (m, 1H), 4.09-4.03 (m, 2H), 4.03-3.90 (m, 2H), 2.80-2.59 (m, 2H), 2.25-2.15 (m, 1H), 2.08 (s, 3H), 1.69-1.59 (m, 2H), 1.39 (s, 9H), 1.32-1.21 (m, 2H), 1.19 (t, J=7.2 Hz, 3H)

### Step B. Procedure for Preparation of tert-butyl 4-(3-ethoxy-1-methyl-3-oxo-propyl)piperidine-1-carboxylate

To a solution of tert-butyl 4-[(E)-3-ethoxy-1-methyl-3-oxo-prop-1-enyl]piperidine-1-carboxylate (5.7 g, 19.17 mmol, 1 equiv.) in EtOH (60 mL) was added PtO₂ (870.47 mg, 3.83 mmol, 0.2 equiv.) under N₂. The suspension was degassed under vacuum and purged with H₂ several times. The mixture was stirred under H₂ (15 psi) at 25° C. for 12 hours. The reaction mixture was filtered, and the filtrate was concentrated under reduced pressure to give tert-butyl 4-(3-ethoxy-1-methyl-3-oxo-propyl)piperidine-1-carboxylate (5.7 g, crude) as a colorless oil.

MS (ESI) m/z: 200.2 [M−100+H]⁺

¹H NMR (400 MHz, DMSO-d₆) δ=4.09-4.02 (m, 2H), 3.97 (d, J=11.6 Hz, 2H), 2.60 (s, 2H), 2.36 (dd, J=5.2, 14.8 Hz, 1H), 2.10-2.00 (m, 1H), 1.82-1.71 (m, 1H), 1.55 (d, J=12.0 Hz, 2H), 1.38 (s, 9H), 1.35-1.26 (m, 1H), 1.17 (t, J=7.2 Hz, 3H), 1.10-0.96 (m, 2H), 0.84 (d, J=6.8 Hz, 3H)

### Step C. Procedure for Preparation of tert-butyl 4-(3-hydroxy-1-methyl-propyl)piperidine-1-carboxylate

To a solution of tert-butyl 4-(3-ethoxy-1-methyl-3-oxo-propyl)piperidine-1-carboxylate (5.7 g, 19.04 mmol, 1 equiv.) in THE (60 mL) was added LiAlH₄ (722.57 mg, 19.04 mmol, 1 equiv.) at 0° C. The mixture was then degassed and purged with N₂ and stirred at 0° C. for 2 hours. The reaction mixture was quenched by addition Na₂SO₄·10H₂O (2 g) at 0° C. and filtered to give a filtrate. The filtrate was extracted with ethyl acetate (100 mL×3). The combined organic phase was washed with brine (100 mL×2), dried with anhydrous Na₂SO₄, filtered, and concentrated under reduced pressure to give a residue. The residue was purified by flash silica gel chromatography (Eluent of 0˜30% ethyl acetate/petroleum ether) to give tert-butyl 4-(3-hydroxy-1-methyl-propyl)piperidine-1-carboxylate (4.2 g, 15.6 mmol, 81.7% yield, 95.6% purity) as a colorless oil.

¹H NMR (400 MHz, DMSO-d₆) δ=4.30 (t, J=5.2 Hz, 1H), 4.02-3.92 (m, 2H), 3.49-3.41 (m, 1H), 3.41-3.34 (m, 1H), 2.73-2.55 (m, 2H), 1.56-1.52 (m, 1H), 1.52-1.47 (m, 2H), 1.38 (s, 9H), 1.34-1.28 (m, 1H), 1.28-1.10 (m, 2H), 1.10-0.95 (m, 2H), 0.78 (d, J=6.8 Hz, 3H)

### Step D. Procedure for Preparation of tert-butyl 4-[3-(3-bromo-2-methyl-phenoxy)-1-methyl-propyl]piperidine-1-carboxylate

A mixture of tert-butyl 4-(3-hydroxy-1-methyl-propyl)piperidine-1-carboxylate (2 g, 7.77 mmol, 1 equiv.), 3-bromo-2-methyl-phenol (1.45 g, 7.77 mmol, 1 equiv.), 3-bromo-2-methyl-phenol (1.45 g, 7.77 mmol, 1 equiv.), and 2-(tributyl-λ5-phosphanylidene)acetonitrile (2.25 g, 9.33 mmol, 1.2 equiv.) in toluene (20 mL) was degassed and purged with N₂ and stirred at 120° C. for 4 hours under N₂ atmosphere. The mixture was concentrated under reduced pressure to give a residue. The residue was purified by reverse-phase HPLC (with 0.1% HCOOH) to give tert-butyl 4-[3-(3-bromo-2-methyl-phenoxy)-1-methyl-propyl]piperidine-1-carboxylate (2.5 g, 5.8 mmol, 75.0% yield, 99.5% purity) as a brown oil.

MS (ESI) m/z: 328.3 [M−100+3]⁺ (⁸⁰Br).

¹H NMR (400 MHz, DMSO-d₆) δ=7.17-7.12 (m, 1H), 7.12-7.06 (m, 1H), 6.98 (d, J=7.6 Hz, 1H), 4.08-3.93 (m, 4H), 2.71-2.54 (m, 2H), 2.22 (s, 3H), 1.88-1.80 (m, 1H), 1.60-1.48 (m, 4H), 1.39 (s, 10H), 1.16-1.00 (m, 2H), 0.87 (d, J=6.4 Hz, 3H)

### Step E. Procedure for Preparation of tert-butyl 4-[(1R)-3-(3-bromo-2-methyl-phenoxy)-1-methyl-propyl]piperidine-1-carboxylate

The compound tert-butyl 4-[3-(3-bromo-2-methyl-phenoxy)-1-methyl-propyl]piperidine-1-carboxylate (2.5 g, 5.86 mmol, 1 equiv.) was purified by prep-HPLC to give tert-butyl 4-[(1R)-3-(3-bromo-2-methyl-phenoxy)-1-methyl-propyl]piperidine-1-carboxylate (0.9 g, 2.0 mmol, 35.2% yield, 97.7% purity) as a brown oil and tert-butyl 4-[(1S)-3-(3-bromo-2-methyl-phenoxy)-1-methyl-propyl]piperidine-1-carboxylate (0.7 g, 1.4 mmol, 25.0% yield, 89.3% purity) as a brown oil.

¹H NMR (400 MHz, DMSO-d₆) δ=7.17-7.12 (m, 1H), 7.12-7.06 (m, 1H), 6.98 (d, J=7.6 Hz, 1H), 4.08-3.90 (m, 4H), 2.77-2.54 (m, 2H), 2.22 (s, 3H), 1.90-1.79 (m, 1H), 1.61-1.47 (m, 4H), 1.38 (s, 10H), 1.17-1.05 (m, 2H), 0.87 (d, J=6.8 Hz, 3H)

### Step F. Procedure for Preparation of 4-[(1R)-3-(3-bromo-2-methyl-phenoxy)-1-methyl-propyl]piperidine

A mixture of tert-butyl 4-[(1R)-3-(3-bromo-2-methyl-phenoxy)-1-methyl-propyl]piperidine-1-carboxylate (0.9 g, 2.11 mmol, 1 equiv.) and HCl/dioxane (4 M, 527.69 μL, 1 equiv.) was stirred at 25° C. for 2 hours. The mixture was filtered to give a filter cake. The crude product was triturated with EtOAc 5 mL at 25° C. for 10 min to give 4-[(1R)-3-(3-bromo-2-methyl-phenoxy)-1-methyl-propyl]piperidine (620 mg, 1.6 mmol, 78.9% yield, 97.4% purity) as a white solid.

MS (ESI) m/z: 326.0 [M+H]⁺

SFC Method details: Column: Chiralpak 1H-3 50×4.6 mm I.D., 3 um; Mobile phase: Phase A for Hexane (0.05% IPAm), and Phase B for IPA (0.05% IPAm); Gradient elution: 10% B in A; Flow rate: 1 mL/min; Detector: PDA; Column Temp: 35° C.;

### Step G. Procedure for Preparation of ethyl 2-[4-[(1R)-3-(3-bromo-2-methyl-phenoxy)-1-methyl-propyl]-1-piperidyl]acetate

To a solution of 4-[(1R)-3-(3-bromo-2-methyl-phenoxy)-1-methyl-propyl]piperidine (620 mg, 1.71 mmol, 1 equiv., HCl) and ethyl 2-bromoacetate (256.90 mg, 1.54 mmol, 170.13 uL, 0.9 equiv.) in MeCN (7 mL) was added K₂CO₃ (708.71 mg, 5.13 mmol, 3 equiv.). The mixture was stirred at 60° C. for 2 hours. The mixture was filtered to give a filtrate, which was concentrated under reduced pressure to give a residue. The residue was purified by flash silica gel chromatography (Eluent of 0-10% methanol/dichloromethane) to give ethyl 2-[4-[(1R)-3-(3-bromo-2-methyl-phenoxy)-1-methyl-propyl]-1-piperidyl]acetate (530 mg, 1.2 mmol, 74.5% yield, 99.1% purity) as a yellow oil.

MS (ESI) m/z: 414.0 [M+3]⁺ (⁸⁰Br).

¹H NMR (400 MHz, DMSO-d₆) δ=7.17-7.13 (m, 1H), 7.12-7.06 (m, 1H), 6.98 (d, J=7.6 Hz, 1H), 4.09-4.04 (m, 2H), 4.04-3.91 (m, 2H), 3.14 (s, 2H), 2.84 (d, J=11.2 Hz, 2H), 2.22 (s, 3H), 2.13-2.04 (m, 2H), 1.90-1.80 (m, 1H), 1.60-1.46 (m, 4H), 1.33-1.20 (m, 2H), 1.20-1.10 (m, 4H), 0.88 (d, J=6.4 Hz, 3H)

### Step H. Procedure for Preparation of tert-butyl 6-[8-(1,3-benzothiazol-2-ylcarbamoyl)-3,4-dihydro-1H-isoquinolin-2-yl]-3-[3-[(3R)-3-[1-(2-ethoxy-2-oxo-ethyl)-4-piperidyl]butoxy]-2-methyl-phenyl]pyridine-2-carboxylate

Ethyl 2-[4-[(1R)-3-(3-bromo-2-methyl-phenoxy)-1-methyl-propyl]-1-piperidyl]acetate (400 mg, 970.03 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (594.19 mg, 970.03 μmol, 1 equiv.), KF (169.07 mg, 2.91 mmol, 68.17 μL, 3 equiv.), and Ad₂nBuP Pd G₃ (70.64 mg, 97.00 μmol, 0.1 equiv.) were taken up into a microwave tube in dioxane (4 mL) and H₂O (0.4 mL). The sealed tube was heated at 100° C. for 1 hour under microwave. The mixture was concentrated under reduced pressure to give a residue. The residue was purified by flash silica gel chromatography (Eluent of 0˜50% ethyl acetate/petroleum ether) to give tert-butyl 6-[8-(1,3-benzothiazol-2-ylcarbamoyl)-3,4-dihydro-1H-isoquinolin-2-yl]-3-[3-[(3R)-3-[1-(2-ethoxy-2-oxo-ethyl)-4-piperidyl]butoxy]-2-methyl-phenyl]pyridine-2-carboxylate (470 mg, 529.1 μmol, 54.5% yield, 92.1% purity) as a yellow solid.

MS (ESI) m/z: 818.8 [M+H]⁺

¹H NMR (400 MHz, DMSO-d₆) δ=12.86 (s, 1H), 8.01 (d, J=7.6 Hz, 1H), 7.78 (d, J=8.0 Hz, 1H), 7.60 (d, J=7.6 Hz, 1H), 7.50-7.39 (m, 3H), 7.39-7.31 (m, 2H), 7.12-7.06 (m, 1H), 6.96-6.88 (m, 2H), 6.57 (d, J=7.6 Hz, 1H), 4.97 (s, 2H), 4.09-4.03 (m, 2H), 4.02-3.92 (m, 2H), 3.87 (t, J=5.6 Hz, 2H), 3.14 (s, 2H), 3.03 (t, J=5.6 Hz, 2H), 2.83 (d, J=10.0 Hz, 2H), 2.13-2.03 (m, 2H), 1.86 (s, 3H), 1.84-1.76 (m, 1H), 1.61-1.44 (m, 4H), 1.33-1.21 (m, 2H), 1.18-1.14 (m, 4H), 1.03-0.97 (m, 9H), 0.86 (d, J=5.6 Hz, 3H)

### Step I. Procedure for preparation of 2-[4-[(1R)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-1-methyl-propyl]-1-piperidyl]acetic acid

To a solution of tert-butyl 6-[8-(1,3-benzothiazol-2-ylcarbamoyl)-3,4-dihydro-1H-isoquinolin-2-yl]-3-[3-[(3R)-3-[1-(2-ethoxy-2-oxo-ethyl)-4-piperidyl]butoxy]-2-methyl-phenyl]pyridine-2-carboxylate (470 mg, 574.55 μmol, 1 equiv.) in THE (4 mL) was added LiOH (1 M, 1.72 mL, 3 equiv.). The mixture was stirred at 25° C. for 12 hours. The mixture was concentrated under reduced pressure to removed THF, added HCl (1 M) to pH=6, and filtered to give 2-[4-[(1R)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-1-methyl-propyl]-1-piperidyl]acetic acid (370 mg, crude) as a yellow solid.

MS (ESI) m/z: 713.2 [M+H]⁺

### Step J. Procedure for Preparation of tert-butyl 6-[8-(1,3-benzothiazol-2-ylcarbamoyl)-3,4-dihydro-1H-isoquinolin-2-yl]-3-[3-[(3R)-3-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylate

To a solution of 2-[4-[(1R)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-1-methyl-propyl]-1-piperidyl]acetic acid (100 mg, 126.59 μmol, 1 equiv.) and HATU (72.20 mg, 189.88 μmol, 1.5 equiv.) in DMF (1 mL) was added DIEA (49.08 mg, 379.76 μmol, 66.15 μL, 3 equiv.) and 3-(6-amino-1-methyl-indazol-3-yl)piperidine-2,6-dione (39.23 mg, 151.90 μmol, 1.2 equiv.). The mixture was stirred at 25° C. for 1 hour. The mixture was added into H₂O (5 mL), filtered to give tert-butyl 6-[8-(1,3-benzothiazol-2-ylcarbamoyl)-3,4-dihydro-1H-isoquinolin-2-yl]-3-[3-[(3R)-3-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylate (110 mg, crude) as a pink solid.

MS (ESI) m/z: 1030.7 [M+H]⁺

### Step K. Procedure for Preparation of 6-[8-(1,3-benzothiazol-2-ylcarbamoyl)-3,4-dihydro-1H-isoquinolin-2-yl]-3-[3-[(3R)-3-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[(3R)-3-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylate (110 mg, 106.77 μmol, 1 equiv.) in DCM (0.5 mL) was added TFA (770.00 mg, 6.75 mmol, 0.5 mL, 63.25 equiv.). The mixture was stirred at 25° C. for 12 hours. The mixture was concentrated under reduced pressure to remove DCM. The residue was purified by prep-HPLC to give 6-[8-(1,3-benzothiazol-2-ylcarbamoyl)-3,4-dihydro-1H-isoquinolin-2-yl]-3-[3-[(3R)-3-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylic acid (34.3 mg, 33.6 μmol, 31.5% yield, 95.4% purity) as a yellow solid.

MS (ESI) m/z: 974.5 [M+H]⁺

¹H NMR (400 MHz, DMSO-d₆) δ=13.16-11.97 (m, 2H), 10.88 (s, 1H), 9.87 (s, 1H), 8.14 (s, 1H), 8.07-8.00 (m, 2H), 7.79 (d, J=8.0 Hz, 1H), 7.63 (d, J=8.8 Hz, 2H), 7.49-7.43 (m, 3H), 7.39-7.32 (m, 2H), 7.21 (dd, J=1.2, 8.8 Hz, 1H), 7.10 (t, J=8.0 Hz, 1H), 6.96 (d, J=8.8 Hz, 1H), 6.90 (d, J=8.4 Hz, 1H), 6.63 (d, J=7.6 Hz, 1H), 4.98 (s, 2H), 4.32 (dd, J=5.2, 9.6 Hz, 1H), 4.04-3.97 (m, 2H), 3.94-3.89 (m, 5H), 3.17 (s, 2H), 3.02 (t, J=5.6 Hz, 2H), 2.99-2.93 (m, 2H), 2.66-2.60 (m, 2H), 2.42-2.24 (m, 2H), 2.19-2.14 (m, 2H), 1.89 (s, 3H), 1.65-1.54 (m, 4H), 1.46-1.35 (m, 2H), 1.30-1.18 (m, 2H), 0.93 (d, J=6.4 Hz, 3H)

### Example 144. Preparation of Compound 167b

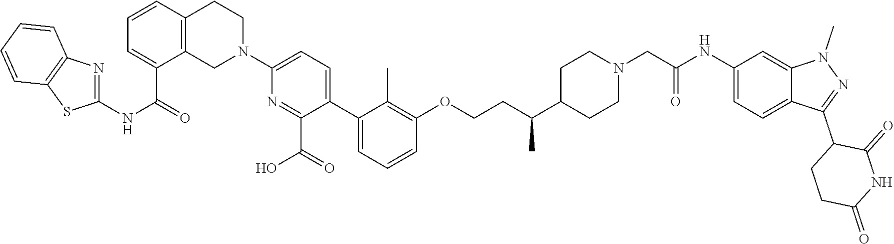 [SMILES: Cc1c(OCC[C@H](C)C2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[(3S)-3-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylic acid

### Step A. Procedure for Preparation of 4-[(1S)-3-(3-bromo-2-methyl-phenoxy)-1-methyl-propyl]piperidine

A mixture of tert-butyl 4-[(1S)-3-(3-bromo-2-methyl-phenoxy)-1-methyl-propyl]piperidine-1-carboxylate (700 mg, 1.64 mmol, 1 equiv.) and HCl/EtOAc (4 M, 7.00 mL, 17.06 equiv.) was stirred at 25° C. for 2 hours. The mixture was concentrated under reduced pressure to give a residue. The residue was triturated with EtOAc (3 mL) at 25° C. for 10 min to give 4-[(1S)-3-(3-bromo-2-methyl-phenoxy)-1-methyl-propyl]piperidine (550 mg, 1.5 mmol, 91.4% yield, 98.9% purity, HCl) as a yellow solid.

MS (ESI) m/z: 327.8 [M+3]⁺(⁸⁰Br).

### Step B. Procedure for Preparation of ethyl 2-[4-[(1S)-3-(3-bromo-2-methyl-phenoxy)-1-methyl-propyl]-1-piperidyl]acetate

To a solution of 4-[(1S)-3-(3-bromo-2-methyl-phenoxy)-1-methyl-propyl]piperidine (550 mg, 1.52 mmol, 1 equiv., HCl) and ethyl 2-bromoacetate (227.90 mg, 1.36 mmol, 150.93 μL, 0.9 equiv.) in CH₃CN (6 mL) was added K₂CO₃ (628.67 mg, 4.55 mmol, 3 equiv.) The mixture was stirred at 60° C. for 1 hour. The mixture was filtered to give a filtrate, which was concentrated under reduced pressure to give a residue. The residue was purified by flash silica gel chromatography (Eluent of 0-10% methanol/dichloromethane) to give ethyl 2-[4-[(1S)-3-(3-bromo-2-methyl-phenoxy)-1-methyl-propyl]-1-piperidyl]acetate (480 mg, 1.2 mmol, 76.3% yield, 99.5% purity) as a yellow oil.

MS (ESI) m/z: 414.0 [M+3]⁺(⁸⁰Br).

¹H NMR (400 MHz, DMSO-d₆) δ=7.17-7.12 (m, 1H), 7.12-7.06 (m, 1H), 6.98 (d, J=8.0 Hz, 1H), 4.10-4.04 (m, 2H), 4.04-3.92 (m, 2H), 3.14 (s, 2H), 2.84 (d, J=11.2 Hz, 2H), 2.22 (s, 3H), 2.14-2.03 (m, 2H), 1.90-1.80 (m, 1H), 1.60-1.46 (m, 4H), 1.34-1.20 (m, 1H), 1.18 (t, J=7.2 Hz, 4H), 0.88 (d, J=6.4 Hz, 3H)

### Step C. Procedure for Preparation of tert-butyl 6-[8-(1,3-benzothiazol-2-ylcarbamoyl)-3,4-dihydro-1H-isoquinolin-2-yl]-3-[3-[(3S)-3-[1-(2-ethoxy-2-oxo-ethyl)-4-piperidyl]butoxy]-2-methyl-phenyl]pyridine-2-carboxylate

Ethyl 2-[4-[(1S)-3-(3-bromo-2-methyl-phenoxy)-1-methyl-propyl]-1-piperidyl]acetate (400 mg, 970.03 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (594.19 mg, 970.03 μmol, 1 equiv.), Ad₂nBuP Pd G₃ (70.64 mg, 97.00 μmol, 0.1 equiv.), and KF (169.07 mg, 2.91 mmol, 68.17 μL, 3 equiv.) were taken up into a microwave tube in dioxane (4 mL) and H₂O (0.4 mL). The sealed tube was heated at 100° C. for 1 hour under microwave. The mixture was concentrated under reduced pressure to give a residue. The residue was purified by flash silica gel chromatography (Eluent of 0˜50% ethyl acetate/petroleum ether) to give tert-butyl 6-[8-(1,3-benzothiazol-2-ylcarbamoyl)-3,4-dihydro-1H-isoquinolin-2-yl]-3-[3-[(3S)-3-[1-(2-ethoxy-2-oxo-ethyl)-4-piperidyl]butoxy]-2-methyl-phenyl]pyridine-2-carboxylate (530 mg, 595.7 μmol, 61.4% yield, 91.9% purity) as a yellow solid.

MS (ESI) m/z: 818.7 [M+H]⁺

¹H NMR (400 MHz, DMSO-d₆) δ=12.85 (s, 1H), 8.01 (d, J=8.0 Hz, 1H), 7.77 (d, J=8.0 Hz, 1H), 7.60 (d, J=7.6 Hz, 1H), 7.50-7.39 (m, 3H), 7.38-7.31 (m, 2H), 7.13-7.06 (m, 1H), 6.96-6.89 (m, 2H), 6.57 (d, J=7.6 Hz, 1H), 4.97 (s, 2H), 4.08-4.03 (m, 2H), 4.02-3.92 (m, 2H), 3.87 (t, J=5.6 Hz, 2H), 3.13 (s, 2H), 3.02 (t, J=5.6 Hz, 2H), 2.83 (d, J=10.0 Hz, 2H), 2.06 (t, J=10.4 Hz, 2H), 1.86 (s, 3H), 1.84-1.75 (m, 1H), 1.60-1.44 (m, 4H), 1.34-1.20 (m, 2H), 1.18-1.14 (m, 4H), 1.03-0.98 (m, 9H), 0.88-0.82 (m, 3H)

### Step D. Procedure for Preparation of 2-[4-[(1S)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-1-methyl-propyl]-1-piperidyl]acetic acid

To a solution of tert-butyl 6-[8-(1,3-benzothiazol-2-ylcarbamoyl)-3,4-dihydro-1H-isoquinolin-2-yl]-3-[3-[(3S)-3-[1-(2-ethoxy-2-oxo-ethyl)-4-piperidyl]butoxy]-2-methyl-phenyl]pyridine-2-carboxylate (530 mg, 647.90 μmol, 1 equiv.) in THE (4 mL) was added LiOH (1 M, 1.94 mL, 3 equiv.). The mixture was stirred at 25° C. for 12 hours. The mixture was concentrated under reduced pressure to remove THF, treated with HCl (1 M) to pH=6, and filtered to give 2-[4-[(1S)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-1-methyl-propyl]-1-piperidyl]acetic acid (500 mg, crude) as a yellow solid.

MS (ESI) m/z: 790.3 [M+H]⁺

### Step E. Procedure for Preparation of tert-butyl 6-[8-(1,3-benzothiazol-2-ylcarbamoyl)-3,4-dihydro-1H-isoquinolin-2-yl]-3-[3-[(3S)-3-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylate

To a solution of 2-[4-[(1S)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-1-methyl-propyl]-1-piperidyl]acetic acid (150 mg, 189.88 μmol, 1 equiv.) and HATU (86.64 mg, 227.85 μmol, 1.2 equiv.) in DMF (1.5 mL) was added DIEA (73.62 mg, 569.64 μmol, 99.22 μL, 3 equiv.) and 3-(6-amino-1-methyl-indazol-3-yl)piperidine-2,6-dione (68.66 mg, 265.83 μmol, 1.4 equiv.). The mixture was stirred at 25° C. for 1 hour. The mixture was added into H₂O (5 mL) and filtered to give a filter cake, which was concentrated under reduced pressure to give a residue. The residue was purified by prep-TLC (SiO₂, DCM:MeOH=10:1) to give tert-butyl 6-[8-(1,3-benzothiazol-2-ylcarbamoyl)-3,4-dihydro-1H-isoquinolin-2-yl]-3-[3-[(3S)-3-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylate (100 mg, 95.0 μmol, 50.1% yield, 97.9% purity) as a yellow solid.

MS (ESI) m/z: 1030.6 [M+H]⁺

### Step F. Procedure for Preparation of 6-[8-(1,3-benzothiazol-2-ylcarbamoyl)-3,4-dihydro-1H-isoquinolin-2-yl]-3-[3-[(3S)-3-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[(3S)-3-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylate (100 mg, 97.06 μmol, 1 equiv.) in DCM (0.5 mL) was added TFA (770.00 mg, 6.75 mmol, 0.5 mL, 69.57 equiv.). The mixture was stirred at 25° C. for 12 hours. The mixture was concentrated under reduced pressure to remove DCM. The residue was purified by prep-HPLC to give 6-[8-(1,3-benzothiazol-2-ylcarbamoyl)-3,4-dihydro-1H-isoquinolin-2-yl]-3-[3-[(3S)-3-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylic acid (33.4 mg, 31.4 μmol, 32.4% yield, 96.1% purity) as an off-white solid.

MS (ESI) m/z: 974.5 [M+H]⁺

¹H NMR (400 MHz, DMSO-d₆) δ=13.01-12.69 (m, 1H), 10.89 (s, 1H), 8.07-7.97 (m, 2H), 7.79 (d, J=8.0 Hz, 1H), 7.69-7.58 (m, 2H), 7.50-7.41 (m, 3H), 7.40-7.32 (m, 2H), 7.17 (d, J=8.8 Hz, 1H), 7.13-7.07 (m, 1H), 6.97 (d, J=8.8 Hz, 1H), 6.91 (d, J=8.4 Hz, 1H), 6.63 (d, J=7.6 Hz, 1H), 4.98 (s, 2H), 4.36-4.30 (m, 1H), 4.06-3.97 (m, 2H), 3.93 (s, 5H), 3.11-2.92 (m, 4H), 2.69-2.60 (m, 2H), 2.40-2.27 (m, 2H), 2.25-2.04 (m, 2H), 1.90 (s, 5H), 1.78-1.21 (m, 8H), 0.93 (d, J=6.4 Hz, 3H)

### Example 145. Preparation of Compound 171a

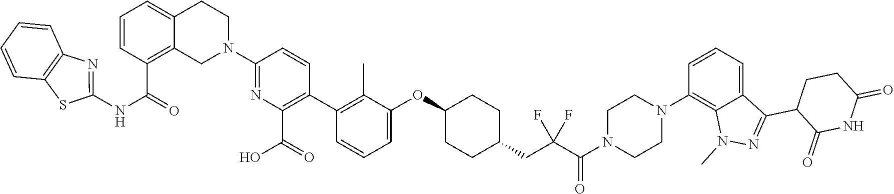 [SMILES: Cc1c(O[C@H]2CC[C@H](CC(F)(F)C(=O)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-7-yl)piperazin-1-yl)-2,2-difluoro-3-oxopropyl)cyclohexyl)oxy)-2-methylphenyl)picolinic acid

### Step A. Procedure for Preparation of 3-[4-(3-bromo-2-methyl-phenoxy)cyclohexyl]-2,2-difluoro-propanoic acid

A mixture of ethyl 3-[4-(3-bromo-2-methyl-phenoxy)cyclohexyl]-2,2-difluoro-propanoate (800 mg, 1.97 mmol, 1 equiv.), LiOH·H₂O (248.51 mg, 5.92 mmol, 3 equiv.), in THE (6 mL) and H₂O (2 mL) was stirred at 40° C. for 16 hours. The reaction mixture was diluted with H₂O (2 mL) and concentrated as a turbid liquid. Then the pH of the turbid liquid was adjusted to 3 with 1 N HCl (0.5 mL), and the residue was extracted with DCM (10 mL), The combined organic layers were filtered and concentrated. The residue was used in the next step without other purification. The compound 3-[4-(3-bromo-2-methyl-phenoxy)cyclohexyl]-2,2-difluoro-propanoic acid (800 mg, crude) was obtained as a yellow oil.

### Step B. Procedure for Preparation of 3-(7-(4-(3-((1r,4r)-4-(3-bromo-2-methylphenoxy)cyclohexyl)-2,2-difluoropropanoyl)piperazin-1-yl)-1-methyl-1H-indazol-3-yl)piperidine-2,6-dione

A mixture of 3-[4-(3-bromo-2-methyl-phenoxy)cyclohexyl]-2,2-difluoro-propanoic acid (100 mg, 265.10 μmol, 1 equiv.), HATU (251.99 mg, 662.74 μmol, 2.5 equiv.), and DIEA (102.79 mg, 795.29 μmol, 138.53 uL, 3 equiv.) in DMF (1 mL) was stirred at 40° C. for 0.25 h. 3-(1-Methyl-7-piperazin-1-yl-indazol-3-yl)piperidine-2,6-dione (104.15 mg, 318.12 μmol, 1.2 equiv.) was then added to the mixture, and the mixture was stirred at 40° C. for 15.75 hours. The reaction mixture was diluted with H₂O (4 mL×3) and extracted with ethyl acetate (8 mL×3). The combined organic layers were filtered and concentrated under reduced pressure to give a residue. The mixture was purified by flash silica gel chromatography (Eluent of 0˜70% ethyl acetate/Petroleum) to give 3-(7-(4-(3-((1r,4r)-4-(3-bromo-2-methylphenoxy)cyclohexyl)-2,2-difluoropropanoyl)piperazin-1-yl)-1-methyl-1H-indazol-3-yl)piperidine-2,6-dione (125 mg, 182.1 μmol, 68.7% yield) as a yellow oil.

MS (ESI) m/z: 686.2 [M+H]⁺.

### Step C. Procedure for Preparation of tert-butyl 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-7-yl)piperazin-1-yl)-2,2-difluoro-3-oxopropyl)cyclohexyl)oxy)-2-methylphenyl)picolinate

A mixture of 3-(7-(4-(3-((1r,4r)-4-(3-bromo-2-methylphenoxy)cyclohexyl)-2,2-difluoropropanoyl)piperazin-1-yl)-1-methyl-1H-indazol-3-yl)piperidine-2,6-dione (125 mg, 182.06 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (133.82 mg, 218.47 μmol, 1.2 equiv.), [2-(2-aminophenyl)phenyl]palladium(1⁺); bis(1-adamantyl)-butyl-phosphane; methanesulfonate (26.52 mg, 36.41 μmol, 0.2 equiv.), and KF (1.5 M, 182.06 μL, 1.5 equiv.) in dioxane (1.5 mL) and H₂O (0.5 mL) was degassed and purged with N₂ three times, and then the mixture was stirred at 100° C. for 1 h under microwave. The combined organic layers were filtered and concentrated under reduced pressure to give a residue. The mixture was purified by flash silica gel chromatography (Eluent of 0˜70% ethyl acetate/petroleum ether) to give tert-butyl 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-7-yl)piperazin-1-yl)-2,2-difluoro-3-oxopropyl)cyclohexyl)oxy)-2-methylphenyl)picolinate (170 mg, 155.6 μmol, 85.5% yield) as a yellow oil.

MS (ESI) m/z: 1092.4 [M+H]⁺.

### Step D. Procedure for Preparation of 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-7-yl)piperazin-1-yl)-2,2-difluoro-3-oxopropyl)cyclohexyl)oxy)-2-methylphenyl)picolinic acid

A solution of tert-butyl 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-7-yl)piperazin-1-yl)-2,2-difluoro-3-oxopropyl)cyclohexyl)oxy)-2-methylphenyl)picolinate (170 mg, 155.64 μmol, 1 equiv.) in DCM (0.8 mL) and TFA (0.8 mL) was stirred at 40° C. for 4 h. The combined organic layers were filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1r,4r)-4-(3-(4-(3-(2,6-dioxopiperidin-3-yl)-1-methyl-1H-indazol-7-yl)piperazin-1-yl)-2,2-difluoro-3-oxopropyl)cyclohexyl)oxy)-2-methylphenyl)picolinic acid (34.9 mg, 32.4 μmol, 20.8% yield, 96.1% purity) as a white solid.

MS (ESI) m/z: 1036.3 [M+H]⁺.

¹H NMR (400 MHz, DMSO-d₆) δ=13.25-12.24 (m, 2H), 10.88 (s, 1H), 8.03 (d, J=8.0 Hz, 1H), 7.79 (d, J=8.0 Hz, 1H), 7.62 (d, J=7.2 Hz, 1H), 7.49-7.43 (m, 4H), 7.39-7.33 (m, 2H), 7.09-7.01 (m, 3H), 6.97-6.91 (m, 2H), 6.62 (d, J=7.2 Hz, 1H), 4.98 (s, 2H), 4.35 (dd, J=4.8, 9.6 Hz, 1H), 4.28 (s, 3H), 4.25-4.13 (m, 2H), 3.93-3.90 (m, 2H), 3.64-3.49 (m, 1H), 3.29-3.09 (m, 4H), 3.04-3.01 (m, 2H), 2.90-2.71 (m, 2H), 2.68-2.62 (m, 2H), 2.36-2.31 (m, 1H), 2.21-2.06 (m, 5H), 1.92-1.85 (m, 5H), 1.74-1.65 (m, 1H), 1.45-1.34 (m, 2H), 1.31-1.23 (m, 2H)

¹⁹F NMR (400 MHz, DMSO-d₆) δ=−95.763 ppm.

### Example 146. Preparation of Compound 174a

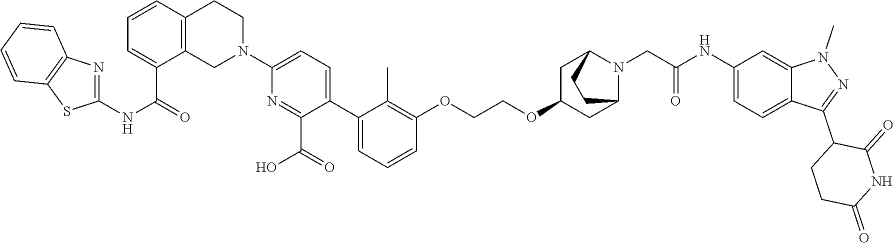 [SMILES: Cc1c(OCCO[C@H]2C[C@H]3CC[C@@H](C2)N3CC(=O)Nc2ccc3c(C4CCC(=O)NC4=O)nn(C)c3c2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(2-(((1R,3r,5S)-8-(2-((3-(2,6-dioxopiperidin-3-yl)-1-methyl-1H-indazol-6-yl)amino)-2-oxoethyl)-8-azabicyclo[3.2.1]octan-3-yl)oxy)ethoxy)-2-methylphenyl)picolinic acid

### Step A. Procedure for Preparation of tert-butyl (1R, 5S)-3-(2-ethoxy-2-oxo-ethoxy)-8-azabicyclo[3.2.1]octane-8-carboxylate

A mixture of (1R, 3r, 5S)-tert-butyl 3-hydroxy-8-azabicyclo[3.2.1]octane-8-carboxylate (2.0 g, 8.80 mmol, 1.0 equiv.), rhodium (II) acetate dimer (194.45 mg, 439.95 μmol, 0.05 equiv.) in DCM (30 mL), ethyl 2-diazoacetate (1.71 g, 14.96 mmol, 1.57 mL, 1.7 equiv.) was stirred at 20° C. for 2 hours under N₂, and then treated with additional ethyl 2-diazoacetate (1.51 g, 13.20 mmol, 1.38 mL, 1.5 equiv.). The mixture was stirred at 20° C. for another 2 hours under N₂, and to the mixture was added ethyl 2-diazoacetate (501.99 mg, 4.40 mmol, 460.54 μL, 0.5 equiv.). The mixture was stirred at 20° C. for 12 hours under N₂. The reaction mixture was filtered under reduced pressure to give a residue. The residue was purified by flash silica gel chromatography (Eluent of 0˜15% ethyl acetate/petroleum ether) to give tert-butyl (1R, 5S)-3-(2-ethoxy-2-oxo-ethoxy)-8-azabicyclo [3.2.1]octane-8-carboxylate (2.46 g, 7.85 mmol, 89.18% yield) as a colorless oil.

¹H NMR (400 MHz, DMSO-d₆) δ=4.12-4.06 (m, 4H), 3.99 (s, 2H), 3.63 (s, 1H), 2.03 (d, J=6.4 Hz, 2H), 1.88-1.78 (m, 5H), 1.39 (s, 8H), 1.24-1.14 (m, 5H)

### Step B. Procedure for Preparation of tert-butyl (1R, 5S)-3-(2-hydroxyethoxy)-8-azabicyclo [3.2.1]octane-8-carboxylate

To a solution of tert-butyl (1R, 5S)-3-(2-ethoxy-2-oxo-ethoxy)-8-azabicyclo[3.2.1]octane-8-carboxylate (800 mg, 2.55 mmol, 1.0 equiv.) in THE (8 mL) was purged with N₂ and added in portions LiAlH₄ (96.89 mg, 2.55 mmol, 1.0 equiv.) at 0° C. The mixture was stirred under N₂ at 0° C. for 2 hours. The reaction mixture was quenched with Na₂SO₄·10H₂O and slowly warmed to 20° C. The mixture was filtered and the filter cake was washed with DCM (8 mL). Then the organic layer were concentrated under reduced pressure to give tert-butyl (1R, 5S)-3-(2-hydroxyethoxy)-8-azabicyclo[3.2.1]octane-8-carboxylate (553 mg, crude) as a yellow oil.

### Step C. Procedure for Preparation of tert-butyl (1R, 5S)-3-[2-(3-bromo-2-methyl-phenoxy) ethoxy]-8-azabicyclo[3.2.1]octane-8-carboxylate

To a solution of tert-butyl (1R, 5S)-3-(2-hydroxyethoxy)-8-azabicyclo[3.2.1]octane-8-carboxylate (1.40 g, 5.16 mmol, 1.0 equiv.) and 3-bromo-2-methyl-phenol (1.93 g, 10.32 mmol, 2.0 equiv.) in toluene (20 mL) was added 2-(tributyl-λ⁵-phosphanylidene) acetonitrile (1.87 g, 7.74 mmol, 1.5 equiv.). The mixture was stirred at 120° C. for 12 hours under N₂ atmosphere. The reaction mixture was concentrated under reduced pressure to remove solvent. The residue was purified by flash silica gel chromatography (Eluent of 0-30% ethyl acetate/petroleum ether) to give compound tert-butyl (1R, 5S)-3-[2-(3-bromo-2-methyl-phenoxy) ethoxy]-8-azabicyclo[3.2.1]octane-8-carboxylate (1.80 g, 3.9 mmol, 76.2% yield, 96.2% purity) as a light yellow oil.

¹H NMR (400 MHz, DMSO-d₆) δ=7.18-7.15 (m, 1H), 7.09 (t, J=8.0 Hz, 1H), 7.00 (d, J=8.0 Hz, 1H), 4.14-4.09 (m, 2H), 3.99 (s, 2H), 3.71-3.67 (m, 3H), 2.24 (s, 3H), 2.04-1.98 (m, 2H), 1.86-1.73 (m, 6H), 1.39 (s, 9H)

### Step D. Procedure for Preparation of ethyl 2-[(1R, 5S)-3-[2-(3-bromo-2-methyl-phenoxy)ethoxy]-8-azabicyclo[3.2.1]octan-8-yl]acetate

To a solution of tert-butyl (1R, 5S)-3-[2-(3-bromo-2-methyl-phenoxy) ethoxy]-8-azabicyclo[3.2.1]octane-8-carboxylate (1.80 g, 4.09 mmol, 1.0 equiv.) was added HCl/EtOAc (15 mL). The mixture was stirred at 25° C. for 1 hour. The reaction mixture was concentrated under reduced pressure to give (1R, 5S)-3-[2-(3-bromo-2-methyl-phenoxy) ethoxy]-8-azabicyclo[3.2.1]octane (1.19 g, crude, HCl) as a white solid.

MS (ESI) m/z: 342.0 [M+H]⁺.

### Step E. Procedure for Preparation of ethyl 2-[(1R, 5S)-3-[2-(3-bromo-2-methyl-phenoxy)ethoxy]-8-azabicyclo[3.2.1]octan-8-yl]acetate

To a solution of (1R, 5S)-3-[2-(3-bromo-2-methyl-phenoxy) ethoxy]-8-azabicyclo[3.2.1]octane (1.19 g, 3.16 mmol, 1.0 equiv. HCl) and ethyl 2-bromoacetate (474.78 mg, 2.84 mmol, 314.43 μL, 0.9 equiv.) in CH₃CN (20 mL) was added K₂CO₃ (2.18 g, 15.79 mmol, 5.0 equiv.). The mixture was stirred at 60° C. for 2 hours. The reaction mixture was quenched by addition H₂O (30 mL) at 10° C., and then diluted with ethyl acetate (30 mL) and extracted with ethyl acetate (25 mL×2). The combined organic layers were washed with brine (25 mL×2), dried over Na₂SO₄, filtered, and concentrated under reduced pressure to give a residue. The residue was purified by flash silica gel chromatography (Eluent of 30˜60% ethyl acetate/petroleum ether) to give ethyl 2-[(1R, 5S)-3-[2-(3-bromo-2-methyl-phenoxy)ethoxy]-8-azabicyclo[3.2.1]octan-8-yl]acetate (912 mg, 2.1 mmol, 67.0% yield, 98.9% purity) as a colorless oil.

MS (ESI) m/z: 428.0 [M+H]⁺.

### Step F. Procedure for Preparation of tert-butyl 6-[8-(1,3-benzothiazol-2-ylcarbamoyl)-3,4-dihydro-1H-isoquinolin-2-yl]-3-[3-[2-[[(1R, 5S)-8-(2-methoxy-2-oxo-ethyl)-8-azabicyclo[3.2.1]octan-3-yl]oxy]ethoxy]-2-methyl-phenyl]pyridine-2-carboxylate

Ethyl 2-[(1R, 5S)-3-[2-(3-bromo-2-methyl-phenoxy)ethoxy]-8-azabicyclo[3.2.1]octan-8-yl]acetate (250 mg, 606.33 μmol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (408.54 mg, 666.96 μmol, 1.1 eq), [2-(2-aminophenyl)phenyl]palladium(1⁺); bis(1-adamantyl)-butyl-phosphane; methanesulfonate (44.16 mg, 60.63 μmol, 0.1 equiv.) and K₃PO₄ (386.11 mg, 1.82 mmol, 3.0 equiv.) were taken up into a microwave tube in H₂O (1 mL) and dioxane (10 mL). The sealed tube was heated at 100° C. for 2 hours under microwave. The reaction mixture was filtered under reduced pressure to give a residue. The residue was purified by flash silica gel chromatography (Eluent of 0˜5% ethyl acetate/Methanol) to give tert-butyl 6-[8-(1,3-benzothiazol-2-ylcarbamoyl)-3,4-dihydro-1H-isoquinolin-2-yl]-3-[3-[2-[[(1R, 5S)-8-(2-methoxy-2-oxo-ethyl)-8-azabicyclo[3.2.1]octan-3-yl]oxy]ethoxy]-2-methyl-phenyl]pyridine-2-carboxylate (212.5 mg, 201.7 μmol, 33.2% yield, 77.6% purity) as a yellow solid.

MS (ESI) m/z: 832.5 [M+H]⁺.

### Step G. Procedure for Preparation of 2-[(1R, 5S)-3-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]ethoxy]-8-azabicyclo[3.2.1]octan-8-yl]acetic acid

To a solution of tert-butyl 6-[8-(1,3-benzothiazol-2-ylcarbamoyl)-3,4-dihydro-1H-isoquinolin-2-yl]-3-[3-[2-[[(1R, 5S)-8-(2-methoxy-2-oxo-ethyl)-8-azabicyclo[3.2.1]octan-3-yl]oxy]ethoxy]-2-methyl-phenyl]pyridine-2-carboxylate (1.4 g, 1.68 mmol, 1.0 equiv.) in MeOH (10 mL) and H₂O (5 mL) was added LiOH·H₂O (353.05 mg, 8.41 mmol, 5.0 equiv.). The reaction mixture was stirred at 40° C. for 1 hour. The reaction mixture was treated with H₂O (5 ml) and adjusted to pH=4˜5 with 1M HCl. Then the reaction mixture concentrated under reduced pressure to give a residue. The compound 2-[(1R, 5S)-3-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]ethoxy]-8-azabicyclo[3.2.1]octan-8-yl]acetic acid (767 mg, crude) was obtained as a yellow solid.

MS (ESI) m/z: 804.2 [M+H]⁺.

### Step H. Procedure for Preparation of tert-butyl 6-[8-(1,3-benzothiazol-2-ylcarbamoyl)-3,4-dihydro-1H-isoquinolin-2-yl]-3-[3-[2-[[(1R,5S)-8-[2-[[3-(2,6-dioxo-3-piperidyl)-1-methyl-indazol-6-yl]amino]-2-oxo-ethyl]-8-azabicyclo[3.2.1]octan-3-yl]oxy]ethoxy]-2-methyl-phenyl]pyridine-2-carboxylate

To a solution of 2-[(1R, 5S)-3-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]ethoxy]-8-azabicyclo[3.2.1]octan-8-yl]acetic acid (120 mg, 149.26 μmol, 1.0 equiv.) and 3-(6-amino-1-methyl-indazol-3-yl)piperidine-2,6-dione (46.26 mg, 179.11 μmol, 1.2 equiv.) in pyridine (2 mL) was added EDCI (42.92 mg, 223.89 μmol, 1.5 equiv.). The mixture was stirred at 25° C. for 16 hours. The reaction mixture was added into H₂O (5 mL). The mixture was triturated with water (5 mL) at 25° C. for 30 minutes and filtered to give tert-butyl 6-[8-(1,3-benzothiazol-2-ylcarbamoyl)-3,4-dihydro-1H-isoquinolin-2-yl]-3-[3-[2-[[(1R,5S)-8-[2-[[3-(2,6-dioxo-3-piperidyl)-1-methyl-indazol-6-yl]amino]-2-oxo-ethyl]-8-azabicyclo[3.2.1]octan-3-yl]oxy]ethoxy]-2-methyl-phenyl]pyridine-2-carboxylate (137 mg, 112.9 μmol, 75.6% yield, 86.4% purity) as a brown solid.

MS (ESI) m/z: 1044.9 [M+H]⁺

### Step I. Procedure for Preparation of 6-(8-(benzo[d]thiazol-2-ylcarbamoyl)-3,4-dihydroisoquinolin-2(1H)-yl)-3-(3-(2-(((1R,3r,5S)-8-(2-((3-(2,6-dioxopiperidin-3-yl)-1-methyl-1H-indazol-6-yl)amino)-2-oxoethyl)-8-azabicyclo[3.2.1]octan-3-yl)oxy)ethoxy)-2-methylphenyl)picolinic acid

To a solution of tert-butyl 6-[8-(1,3-benzothiazol-2-ylcarbamoyl)-3,4-dihydro-1H-isoquinolin-2-yl]-3-[3-[2-[[(1R,5S)-8-[2-[[3-(2,6-dioxo-3-piperidyl)-1-methyl-indazol-6-yl]amino]-2-oxo-ethyl]-8-azabicyclo[3.2.1]octan-3-yl]oxy]ethoxy]-2-methyl-phenyl]pyridine-2-carboxylate (137 mg, 131.20 μmol, 1.0 equiv.) in DCM (1 mL) and TFA (1 mL), the mixture was stirred at 25° C. for 16 hours. The mixture was concentrated to remove DCM. The residue was purified by prep-HPLC to give 6-(8-(benzo[d]thiazol-2-ylcarbamoyl)-3,4-dihydroisoquinolin-2(1H)-yl)-3-(3-(2-(((1R,3r,5S)-8-(2-((3-(2,6-dioxopiperidin-3-yl)-1-methyl-1H-indazol-6-yl)amino)-2-oxoethyl)-8-azabicyclo[3.2.1]octan-3-yl)oxy)ethoxy)-2-methylphenyl)picolinic acid (32.8 mg, 32.2 μmol, 24.5% yield, 96.9% purity) as a white solid.

MS (ESI) m/z: 988.6 [M+H]⁺

¹H NMR (400 MHz, DMSO-d₆) δ=10.86 (s, 1H), 9.92 (s, 1H), 8.16 (s, 1H), 8.05-7.98 (m, 2H), 7.77 (d, J=8.0 Hz, 1H), 7.65-7.60 (m, 2H), 7.47-7.41 (m, 3H), 7.38-7.31 (m, 2H), 7.24 (dd, J=1.6, 8.8 Hz, 1H), 7.09 (t, J=8.0 Hz, 1H), 6.96-6.88 (m, 2H), 6.65 (d, J=7.6 Hz, 1H), 4.97 (s, 2H), 4.32 (dd, J=5.2, 9.6 Hz, 1H), 4.08 (s, 2H), 3.93-3.89 (m, 5H), 3.69 (d, J=4.4 Hz, 2H), 3.62 (s, 2H), 3.08 (s, 2H), 3.02 (t, J=5.6 Hz, 2H), 2.70-2.60 (m, 2H), 2.35-2.30 (m, 1H), 2.20-2.10 (m, 1H), 2.07-1.99 (m, 5H), 1.91 (s, 3H), 1.83 (d, J=14.0 Hz, 4H).

### Example 147. Preparation of Compound 176a

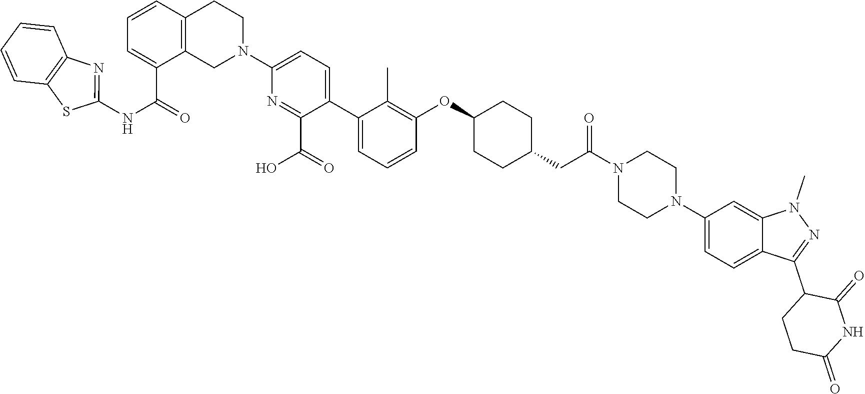 [SMILES: Cc1c(O[C@H]2CC[C@H](CC(=O)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-(2-(4-(3-(2,6-dioxopiperidin-3-yl)-1-methyl-1H-indazol-6-yl)piperazin-1-yl)-2-oxoethyl)cyclohexyl)oxy)-2-methylphenyl)picolinic acid

### Step A. Procedure for Preparation of methyl 2-(4-hydroxycyclohexyl) acetate

To a solution of 2-(4-hydroxycyclohexyl) acetic acid (2 g, 12.64 mmol, 1.0 equiv.) in MeOH (20 mL) was added H₂SO₄ (124.00 mg, 1.26 mmol, 67.39 μL, 0.1 equiv.). The mixture was stirred at 70° C. for 3 hours under N₂ atmosphere. The reaction was diluted with water (20 mL) and extracted with DCM (50 mL×2). The combined organic layers were washed with brine (50 mL×1), dried over Na₂SO₄, filtered, and concentrated under reduced pressure to give a residue. It was used in the next step without further purification to give methyl 2-(4-hydroxycyclohexyl) acetate (2 g, 11.6 mmol, 91.8% yield) as a white oil.

### Step B. Procedure for Preparation of methyl 2-(4-(3-bromo-2-methylphenoxy) cyclohexyl) acetate

To a solution of methyl 2-(4-hydroxycyclohexyl) acetate (2 g, 11.61 mmol, 1.0 equiv.) in toluene (20 mL) was added 2-(tributyl-λ5-phosphanylidene) acetonitrile (4.20 g, 17.42 mmol, 1.5 equiv.) and 3-bromo-2-methylphenol (2.61 g, 13.94 mmol, 1.2 equiv.). The mixture was degassed and purged with N₂ and stirred at 120° C. for 2 hours under N₂ atmosphere. The reaction mixture was concentrated under reduced pressure to remove solvent. The residue was purified by flash silica gel chromatography (Eluent of 0˜15% ethyl acetate/petroleum ether) to give methyl 2-(4-(3-bromo-2-methylphenoxy) cyclohexyl) acetate (3.8 g, 10.9 mmol, 94.0% yield, 98.0% purity) as a white oil.

¹H NMR (400 MHz, CDCl₃-d) δ=7.08-7.03 (m, 1H), 6.89 (t, J=8.0 Hz, 1H), 6.75-6.66 (m, 1H), 4.47 (s, 1H), 3.61 (s, 3H), 2.28-2.15 (m, 5H), 2.09-1.93 (m, 2H), 1.83-1.77 (m, 1H), 1.51 (d, J=13.2 Hz, 4H), 1.44-1.32 (m, 2H)

### Step C. Procedure for Preparation of methyl 2-((1r,4r)-4-(3-bromo-2-methylphenoxy) cyclohexyl) acetate

The methyl 2-(4-(3-bromo-2-methylphenoxy) cyclohexyl) acetate residue was purified by prep-HPLC to give methyl 2-((1r,4r)-4-(3-bromo-2-methylphenoxy) cyclohexyl) acetate (200 mg, 586.10 μmol, 11.11% yield) as a yellow oil.

¹H NMR (400 MHz, CDCl₃-d) 6=7.06 (d, J=8.0 Hz, 1H), 6.89 (t, J=8.0 Hz, 1H), 6.72 (d, J=8.0 Hz, 1H), 4.02 (tt, J=4.4, 10.6 Hz, 1H), 3.61 (s, 3H), 2.22 (s, 3H), 2.17 (d, J=6.4 Hz, 2H), 2.11-2.01 (m, 2H), 1.82-1.77 (m, 2H), 1.54 (s, 1H), 1.48-1.37 (m, 2H), 1.10-0.99 (m, 2H)

### Step D. Procedure for Preparation of tert-butyl 6-(8-(benzo[d]thiazol-2-ylcarbamoyl)-3,4-dihydroisoquinolin-2(1H)-yl)-3-(3-(((1r,4r)-4-(2-methoxy-2-oxoethyl)cyclohexyl)oxy)-2-methylphenyl)picolinate

To a solution of tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl) picolinate (195.82 mg, 319.69 μmol, 1.0 equiv.) in dioxane (2 mL) was added Ad2nBuP Pd G₃ (23.28 mg, 31.97 μmol, 0.1 equiv.), KF (1.5 M, 639.38 μL, 3.0 equiv.), and methyl 2-((1r,4r)-4-(3-bromo-2-methylphenoxy) cyclohexyl) acetate (120 mg, 351.66 μmol, 1.1 equiv.). The mixture was stirred at 100° C. for 1 hour. The reaction was concentrated under reduced pressure to give a residue. The residue was purified by flash silica gel chromatography (Eluent of 0˜30% ethyl acetate/petroleum ether) to give tert-butyl 6-(8-(benzo[d]thiazol-2-ylcarbamoyl)-3,4-dihydroisoquinolin-2(1H)-yl)-3-(3-(((1r,4r)-4-(2-methoxy-2-oxoethyl)cyclohexyl)oxy)-2-methylphenyl)picolinate (200 mg, 267.7 μmol, 83.7% yield) as a yellow oil.

MS (ESI) m/z: 747.8 [M+H]⁺.

### Step E. Procedure for Preparation of 2-((1r,4r)-4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)cyclohexyl)acetic acid

To a solution of tert-butyl 6-(8-(benzo[d]thiazol-2-ylcarbamoyl)-3,4-dihydroisoquinolin-2(1H)-yl)-3-(3-(((1r,4r)-4-(2-methoxy-2-oxoethyl)cyclohexyl)oxy)-2-methylphenyl)picolinate (190 mg, 254.38 μmol, 1.0 equiv.) in THE (2 mL) was added LiOH·H₂O (32.02 mg, 763.14 μmol, 3.0 equiv.) and H₂O (0.5 mL). The mixture was stirred at 25° C. for 12 hours. The reaction mixture was acidified to pH=5-6 with citric acid, filtered, and concentrated under reduced pressure to give a residue. 2-((1r,4r)-4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)cyclohexyl)acetic acid (150 mg, 204.67 μmol, 80.46% yield) was provided as a yellow solid. It was used in the next step without further purification.

MS (ESI) m/z: 733.4 [M+H]⁺.

### Step F. Procedure for Preparation of tert-butyl 6-(8-(benzo[d]thiazol-2-ylcarbamoyl)-3,4-dihydroisoquinolin-2(1H)-yl)-3-(3-(((1r,4r)-4-(2-(4-(3-(2,6-dioxopiperidin-3-yl)-1-methyl-1H-indazol-6-yl)piperazin-1-yl)-2-oxoethyl)cyclohexyl)oxy)-2-methylphenyl)picolinate

To a solution of 2-((1r,4r)-4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)cyclohexyl)acetic acid (40 mg, 54.58 μmol, 1.0 equiv.) in DMF (1 mL) was added HATU (62.26 mg, 163.74 μmol, 3.0 equiv.), DIEA (10.58 mg, 81.87 μmol, 14.26 μL, 1.5 equiv.), and 3-(1-methyl-6-(piperazin-1-yl)-1H-indazol-3-yl)piperidine-2,6-dione (19.65 mg, 60.04 μmol, 1.1 equiv.). The mixture was stirred at 50° C. for 1 hour. The reaction mixture was concentrated under reduced pressure to remove solvent. The residue was purified by prep-TLC (SiO₂, petroleum ether/ethyl acetate=0/1) to give tert-butyl 6-(8-(benzo[d]thiazol-2-ylcarbamoyl)-3,4-dihydroisoquinolin-2(1H)-yl)-3-(3-(((1r,4r)-4-(2-(4-(3-(2,6-dioxopiperidin-3-yl)-1-methyl-1H-indazol-6-yl)piperazin-1-yl)-2-oxoethyl)cyclohexyl)oxy)-2-methylphenyl)picolinate (55 mg, 52.8 μmol, 96.7% yield) as a yellow oil.

MS (ESI) m/z: 1043.5 [M+H]⁺.

### Step G. Procedure for Preparation of 6-(8-(benzo[d]thiazol-2-ylcarbamoyl)-3,4-dihydroisoquinolin-2(1H)-yl)-3-(3-(((1r,4r)-4-(2-(4-(3-(2,6-dioxopiperidin-3-yl)-1-methyl-1H-indazol-6-yl)piperazin-1-yl)-2-oxoethyl)cyclohexyl)oxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(((1r,4r)-4-(2-(4-(3-(2,6-dioxopiperidin-3-yl)-1-methyl-1H-indazol-6-yl)piperazin-1-yl)-2-oxoethyl)cyclohexyl)oxy)-2-methylphenyl)picolinate (70 mg, 67.16 μmol, 1.0 equiv.) in DCM (1 mL) was added TFA (7.66 mg, 67.16 μmol, 4.97 μL, 1.0 equiv.). The mixture was stirred at 40° C. for 4 hours. The reaction mixture was concentrated under reduced pressure to remove solvent. The residue was purified by prep-HPLC to give 6-(8-(benzo[d]thiazol-2-ylcarbamoyl)-3,4-dihydroisoquinolin-2(1H)-yl)-3-(3-(((1r,4r)-4-(2-(4-(3-(2,6-dioxopiperidin-3-yl)-1-methyl-1H-indazol-6-yl)piperazin-1-yl)-2-oxoethyl)cyclohexyl)oxy)-2-methylphenyl)picolinic acid (25.0 mg, 24.8 μmol, 36.9% yield, 97.7% purity) as a yellow solid.

MS (ESI) m/z: 986.4 [M+H]⁺.

¹H NMR (400 MHz, DMSO-d₆) δ=12.92-12.81 (m, 1H), 12.76-12.31 (m, 1H), 10.86 (s, 1H), 8.04 (d, J=8.0 Hz, 1H), 7.80 (d, J=8.0 Hz, 1H), 7.69-7.59 (m, 1H), 7.53 (d, J=8.8 Hz, 1H), 7.50-7.43 (m, 3H), 7.41-7.33 (m, 2H), 7.12-7.04 (m, 1H), 6.99-6.88 (m, 4H), 6.62 (d, J=7.6 Hz, 1H), 4.98 (s, 2H), 4.27 (dd, J=5.2, 9.2 Hz, 1H), 4.24-4.15 (m, 1H), 3.92 (s, 2H), 3.90 (s, 3H), 3.65 (s, 4H), 3.24 (d, J=4.0 Hz, 2H), 3.19 (d, J=1.6 Hz, 2H), 3.03 (t, J=5.6 Hz, 2H), 2.61 (t, J=6.0 Hz, 2H), 2.40-2.22 (m, 4H), 2.16 (dd, J=5.6, 12.7 Hz, 1H), 2.12-2.05 (m, 2H), 1.88 (s, 3H), 1.82 (d, J=13.2 Hz, 2H), 1.44-1.34 (m, 2H), 1.22-1.12 (m, 2H)

### Example 148. Preparation of Compound 177a

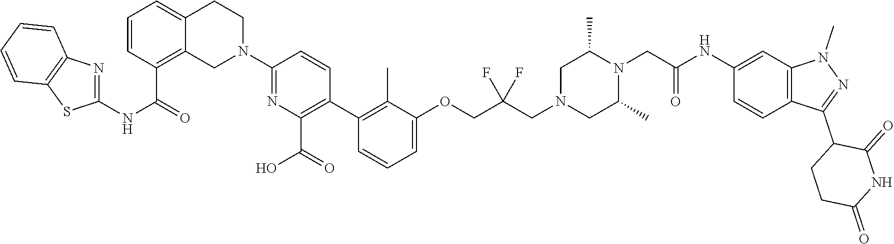 [SMILES: Cc1c(OCC(F)(F)CN2C[C@@H](C)N(CC(=O)Nc3ccc4c(C5CCC(=O)NC5=O)nn(C)c4c3)[C@@H](C)C2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[(3R,5S)-4-[2-[[3-(2,6-dioxo-3-piperidyl)-1-methyl-indazol-6-yl]amino]-2-oxo-ethyl]-3,5-dimethyl-piperazin-1-yl]-2,2-difluoro-propoxy]-2-methyl-phenyl]pyridine-2-carboxylic acid

### Step A. Procedure for Preparation of 3-[tert-butyl(diphenyl)silyl]oxy-2,2-difluoro-propan-1-ol

To a solution of 2,2-difluoropropane-1,3-diol (12.0 g, 107.07 mmol, 1.0 equiv.) in THE (120 mL) was added NaH (4.71 g, 117.78 mmol, 60% purity, 1.1 equiv.) in portions under N₂ at 0° C. The reaction mixture was degassed and purged with N₂ and stirred at 25° C. for 1 hour under N₂ atmosphere. Tert-butyl-chloro-diphenyl-silane (32.37 g, 117.78 mmol, 30.25 mL, 1.1 equiv.) was added to the mixture, and then the mixture was stirred under N₂ at 25° C. for 15 hours. The reaction mixture was quenched by addition NH₄Cl (200 mL) at 0° C., and extracted with ethyl acetate (300 mL×3). The combined organic layers were washed with brine (300 mL), dried over Na₂SO₄, filtered, and concentrated under reduced pressure to give a residue. The residue was purified by flash silica gel chromatography (Eluent of 0˜15% ethyl acetate/petroleum ether) to give 3-[tert-butyl(diphenyl)silyl]oxy-2,2-difluoro-propan-1-ol (42.0 g, crude) was obtained as colorless oil

¹H NMR (400 MHz, CDCl₃-d) δ=7.68 (d, J=6.4 Hz, 4H), 7.50-7.38 (m, 6H), 4.00-3.86 (m, 4H), 1.88-1.76 (m, 1H), 1.08 (s, 9H).

### Step B. Procedure for Preparation of [3-(3-bromo-2-methyl-phenoxy)-2,2-difluoro-propoxy]-tert-butyl-diphenyl-silane

A mixture of 3-bromo-2-methyl-phenol (10.0 g, 53.47 mmol, 1.0 equiv.), 3-[tert-butyl(diphenyl)silyl]oxy-2,2-difluoro-propan-1-ol (22.49 g, 64.16 mmol, 1.2 equiv.), 2-(tributyl-λ5-phosphanylidene)acetonitrile (15.49 g, 64.16 mmol, 1.2 equiv.) in toluene (200 mL) was degassed and purged with N₂ and stirred at 120° C. for 16 hours under N₂ atmosphere. The reaction mixture was concentrated under reduced pressure to remove solvent. The residue was purified by flash silica gel chromatography (Eluent of 0˜5% ethyl acetate/petroleum ether) to give [3-(3-bromo-2-methyl-phenoxy)-2, 2-difluoro-propoxy]-tert-butyl-diphenyl-silane (19.0 g, 36.5 mmol, 68.4% yield) as colorless oil.

¹H NMR (400 MHz, DMSO-d₆) δ=7.57 (d, J=7.2 Hz, 4H), 7.50-7.45 (m, 2H), 7.42-7.37 (m, 4H), 7.25 (d, J=7.2 Hz, 1H), 7.15-7.08 (m, 2H), 4.44 (t, J=12.0 Hz, 2H), 4.02 (t, J=12.8 Hz, 2H), 2.11 (s, 3H), 0.97 (s, 9H).

### Step C. Procedure for Preparation of 3-(3-bromo-2-methyl-phenoxy)-2, 2-difluoro-propan-1-ol

A mixture of [3-(3-bromo-2-methyl-phenoxy)-2,2-difluoro-propoxy]-tert-butyl-diphenyl-silane (19.0 g, 36.57 mmol, 1.0 equiv.) and TBAF (1 M, 73.15 mL, 2.0 equiv.) in THE (150 mL), and then the mixture was stirred at 25° C. for 3 hours. The reaction mixture was quenched by addition H₂O (200 mL) at 25° C., and then diluted with ethyl acetate (200 mL) and extracted with ethyl acetate (200 mL×1). The combined organic layers were washed with H₂O (200 mL×2), dried over Na₂SO₄, filtered and concentrated under reduced pressure to give a residue. The residue was purified by flash silica gel chromatography (Eluent of 0˜12% ethyl acetate/petroleum ether) to give 3-(3-bromo-2-methyl-phenoxy)-2, 2-difluoro-propan-1-ol (7.2 g, 25.6 mmol, 70.0% yield) was obtained as light yellow oil.

¹H NMR (400 MHz, DMSO-d₆) δ=7.23 (d, J=8.0 Hz, 1H), 7.13 (t, J=8.0 Hz, 1H), 7.08-7.04 (m, 1H), 5.67 (t, J=6.4 Hz, 1H), 4.33 (t, J=12.8 Hz, 2H), 3.79 (dt, J=6.0, 13.6 Hz, 2H), 3.17 (s, 1H), 2.25 (s, 3H).

### Step D. Procedure for Preparation of 3-(3-bromo-2-methyl-phenoxy)-2, 2-difluoro-propanal

To a solution of (COCl)₂ (4.52 g, 35.58 mmol, 3.11 mL, 2.0 equiv.) in DCM (100 mL) was added DMSO (5.56 g, 71.15 mmol, 5.56 mL, 4.0 equiv.) in DCM (20 mL) under −70° C. The solution was stirred for 1 hour. 3-(3-Bromo-2-methyl-phenoxy)-2,2-difluoro-propan-1-ol (5.0 g, 17.79 mmol, 1.0 equiv.) in DCM (20 mL) was added into the mixture and stirred for 1 hour. TEA (10.80 g, 106.73 mmol, 14.85 mL, 6.0 equiv.) in DCM (10 mL) was added into the mixture and stirred at −70° C. for 2 hours. The reaction mixture was quenched by addition H₂O (100 mL) at 0° C., and then diluted with DCM (200 mL) and extracted with DCM (100 mL×2). The combined organic layers were dried over Na₂SO₄, filtered and concentrated under reduced pressure to give 3-(3-bromo-2-methyl-phenoxy)-2, 2-difluoro-propanal (5.0 g, crude) as yellow oil, was used into the next step without further purification.

### Step E. Procedure for Preparation of ethyl 2-[(2R,6S)-4-[3-(3-bromo-2-methyl-phenoxy)-2,2-difluoro-propyl]-2,6-dimethyl-piperazin-1-yl]acetate

To a solution of 3-(3-bromo-2-methyl-phenoxy)-2,2-difluoro-propanal (5.0 g, 17.92 mmol, crude purity, 1.0 equiv.), ethyl 2-[(2R,6S)-2,6-dimethylpiperazin-1-yl]acetate (5.09 g, 21.50 mmol, 1.2 equiv., HCl), 4A MS (2.0 g) in DCM (150 mL). The mixture was stirred at 25° C. for 1 hour, was added sodium triacetoxyborohydride (15.19 g, 71.66 mmol, 4.0 equiv.). The mixture was stirred at 25° C. for 19 hours. The reaction mixture was quenched by addition water (200 mL) at 25° C., and then diluted with DCM (100 mL) and extracted with DCM 200 mL (200 mL×1). The combined organic layers were concentrated under reduced pressure to give a residue. The residue was purified by flash silica gel chromatography (Eluent of 0-20% ethyl acetate/petroleum ether) to give ethyl 2-[(2R,6S)-4-[3-(3-bromo-2-methyl-phenoxy)-2,2-difluoro-propyl]-2,6-dimethyl-piperazin-1-yl]acetate (0.9 g, 1.8 mmol, 10.3% yield, 95% purity) as light brown oil.

¹H NMR (400 MHz, CDCl₃) δ=7.22 (d, J=7.6 Hz, 1H), 7.03 (t, J=8.0 Hz, 1H), 6.82 (d, J=8.0 Hz, 1H), 4.25 (t, J=11.6 Hz, 2H), 4.16 (q, J=7.2 Hz, 2H), 3.54 (s, 2H), 2.97 (s, 2H), 2.85 (t, J=13.6 Hz, 2H), 2.74 (d, J=10.8 Hz, 2H), 2.33 (s, 3H), 2.16 (t, J=10.8 Hz, 2H), 1.27 (t, J=7.2 Hz, 3H), 1.04 (d, J=6.4 Hz, 6H)

### Step F. Procedure for Preparation of tert-butyl 6-[8-(1,3-benzothiazol-2-ylcarbamoyl)-3,4-dihydro-1H-isoquinolin-2-yl]-3-[3-[3-[(3R,5S)-4-(2-ethoxy-2-oxo-ethyl)-3,5-dimethyl-piperazin-1-yl]-2,2-difluoro-propoxy]-2-methyl-phenyl]pyridine-2-carboxylate

Ethyl 2-[(2R,6S)-4-[3-(3-bromo-2-methyl-phenoxy)-2,2-difluoro-propyl]-2,6-dimethyl-piperazin-1-yl]acetate (900 mg, 1.94 mmol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (1.25 g, 2.04 mmol, 1.05 equiv.), K₃PO₄ (1.24 g, 5.83 mmol, 3.0 equiv.), and Ad₂nBuP Pd G₃ (282.91 mg, 388.47 μmol, 0.2 equiv.) were taken up into a microwave tube in dioxane (8 mL) and H₂O (0.5 mL). The sealed tube was heated at 100° C. for 2 hours under microwave. The crude reaction mixture was concentrated in vacuo to give a residue. The residue was purified by flash silica gel chromatography (Eluent of 0-45% ethyl acetate/petroleum ether) to give tert-butyl 6-[8-(1,3-benzothiazol-2-ylcarbamoyl)-3,4-dihydro-1H-isoquinolin-2-yl]-3-[3-[3-[(3R,5S)-4-(2-ethoxy-2-oxo-ethyl)-3, 5-dimethyl-piperazin-1-yl]-2,2-difluoro-propoxy]-2-methyl-phenyl]pyridine-2-carboxylate (1.1 g, 1.2 mmol, 63.3% yield, 97.1% purity) was obtained as a yellow solid.

MS (ESI) m/z: 869.4 [M+H]⁺.

### Step G. Procedure for Preparation of 2-[(2R,6S)-4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-2,2-difluoro-propyl]-2,6-dimethyl-piperazin-1-yl]acetic acid

To a solution of tert-butyl 6-[8-(1,3-benzothiazol-2-ylcarbamoyl)-3,4-dihydro-1H-isoquinolin-2-yl]-3-[3-[3-[(3R,5S)-4-(2-ethoxy-2-oxo-ethyl)-3, 5-dimethyl-piperazin-1-yl]-2,2-difluoro-propoxy]-2-methyl-phenyl]pyridine-2-carboxylate (1.0 g, 1.15 mmol, 1.0 equiv.) in H₂O (1 mL) and MeOH (5 mL), was added LiOH·H₂O (241.44 mg, 5.75 mmol, 5.0 equiv.). The mixture was stirred at 25° C. for 16 hours. The reaction mixture was concentrated under reduced pressure to remove MeOH. The residue was diluted with H₂O (5 mL) and adjust pH=4 by citric acid, extracted with DCM (100 mL×2). The combined organic layers were dried over Na₂SO₄, filtered and concentrated under reduced pressure to give 2-[(2R,6S)-4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-2,2-difluoro-propyl]-2,6-dimethyl-piperazin-1-yl]acetic acid (870 mg, 1.0 mmol, 89.9% yield) as a yellow solid.

MS (ESI) m/z: 841.4 [M+H]⁺.

### Step H. Procedure for Preparation of tert-butyl 6-[8-(1,3-benzothiazol-2-ylcarbamoyl)-3,4-dihydro-1H-isoquinolin-2-yl]-3-[3-[3-[(3R,5S)-4-[2-[[3-(2,6-dioxo-3-piperidyl)-1-methyl-indazol-6-yl]amino]-2-oxo-ethyl]-3,5-dimethyl-piperazin-1-yl]-2,2-difluoro-propoxy]-2-methyl-phenyl]pyridine-2-carboxylate

To a solution of 2-[(2R,6S)-4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-2,2-difluoro-propyl]-2,6-dimethyl-piperazin-1-yl]acetic acid (130 mg, 154.58 μmol, 1.0 equiv.) and 3-(6-amino-1-methyl-indazol-3-yl)piperidine-2,6-dione (51.90 mg, 200.96 μmol, 1.3 equiv.) in DMF (2 mL) was added HATU (88.17 mg, 231.87 μmol, 1.5 eq) and DIEA (99.89 mg, 772.91 μmol, 134.62 μL, 5.0 equiv.). The mixture was stirred at 25° C. for 16 hours. The mixture was added into H₂O (5.0 ml) and filtered to give tert-butyl 6-[8-(1,3-benzothiazol-2-ylcarbamoyl)-3,4-dihydro-1H-isoquinolin-2-yl]-3-[3-[3-[(3R,5S)-4-[2-[[3-(2,6-dioxo-3-piperidyl)-1-methyl-indazol-6-yl]amino]-2-oxo-ethyl]-3,5-dimethyl-piperazin-1-yl]-2,2-difluoro-propoxy]-2-methyl-phenyl]pyridine-2-carboxylate (166 mg, 153.5 μmol, 99.3% yield) as a brown solid.

MS (ESI) m/z: 1081.7 [M+H]⁺.

### Step I. Procedure for preparation of 6-[8-(1,3-benzothiazol-2-ylcarbamoyl)-3,4-dihydro-1H-isoquinolin-2-yl]-3-[3-[3-[(3R,5S)-4-[2-[[3-(2,6-dioxo-3-piperidyl)-1-methyl-indazol-6-yl]amino]-2-oxo-ethyl]-3,5-dimethyl-piperazin-1-yl]-2,2-difluoro-propoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[3-[(3R,5S)-4-[2-[[3-(2,6-dioxo-3-piperidyl)-1-methyl-indazol-6-yl]amino]-2-oxo-ethyl]-3,5-dimethyl-piperazin-1-yl]-2,2-difluoro-propoxy]-2-methyl-phenyl]pyridine-2-carboxylate (166 mg, 153.53 μmol, 1 equiv.) in TFA (1 mL) and DCM (1 mL). The mixture was stirred at 25° C. for 16 hours. The mixture was concentrated to remove DCM. The residue was purified by prep-HPLC to give 6-[8-(1,3-benzothiazol-2-ylcarbamoyl)-3,4-dihydro-1H-isoquinolin-2-yl]-3-[3-[3-[(3R,5S)-4-[2-[[3-(2,6-dioxo-3-piperidyl)-1-methyl-indazol-6-yl]amino]-2-oxo-ethyl]-3,5-dimethyl-piperazin-1-yl]-2,2-difluoro-propoxy]-2-methyl-phenyl]pyridine-2-carboxylic acid (53.6 mg, 48.9 μmol, 31.8% yield, 93.5% purity) as a yellow solid.

MS (ESI) m/z: 513.4 [M12+H]⁺.

¹H NMR (400 MHz, DMSO-d₆) δ=12.94-12.12 (m, 1H), 10.88 (s, 1H), 9.77 (s, 1H), 8.14 (s, 1H), 8.02 (d, 2H), 7.81 (d, J=8.0 Hz, 1H), 7.63 (t, J=8.0 Hz, 2H), 7.54-7.47 (m, 3H), 7.37-7.34 (m, 2H), 7.21 (d, J=1.2, 8.8 Hz, 1H), 7.16-7.11 (m, 1H), 7.00 (t, J=8.4 Hz, 2H), 6.72 (d, J=7.6 Hz, 1H), 4.98 (s, 2H), 4.35-4.32 (m, 3H), 3.92-3.90 (m, 5H), 3.02 (t, J=5.6 Hz, 2H), 3.00-2.91 (m, 3H), 2.83-2.71 (m, 4H), 2.70-2.61 (m, 2H), 2.38-2.32 (m, 1H), 2.20-2.14 (m, 3H), 1.93 (s, 3H), 0.95 (d, J=6.0 Hz, 6H)

¹⁹F NMR (400 MHz, DMSO-d₆) δ=106.40

### Example 149. Preparation of Compound 179a

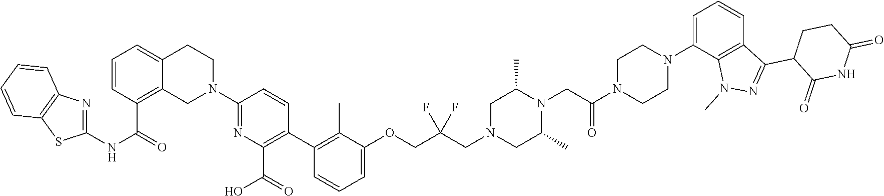 [SMILES: Cc1c(OCC(F)(F)CN2C[C@@H](C)N(CC(=O)N3CCN(c4cccc5c(C6CCC(=O)NC6=O)nn(C)c45)CC3)[C@@H](C)C2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[(3R,5S)-4-[2-[4-[3-(2,6-dioxo-3-piperidyl)-1-methyl-indazol-7-yl]piperazin-1-yl]-2-oxo-ethyl]-3, 5-dimethyl-piperazin-1-yl]-2,2-difluoro-propoxy]-2-methyl-phenyl]pyridine-2-carboxylic acid

### Step A. Procedure for Preparation of tert-butyl 6-[8-(1,3-benzothiazol-2-ylcarbamoyl)-3,4-dihydro-1H-isoquinolin-2-yl]-3-[3-[3-[(3R,5S)-4-[2-[4-[3-(2,6-dioxo-3-piperidyl)-1-methyl-indazol-7-yl]piperazin-1-yl]-2-oxo-ethyl]-3,5-dimethyl-piperazin-1-yl]-2,2-difluoro-propoxy]-2-methyl-phenyl]pyridine-2-carboxylate

To a solution of 2-[(2R,6S)-4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-2,2-difluoro-propyl]-2,6-dimethyl-piperazin-1-yl]acetic acid (120 mg, 142.69 μmol, 1.0 equiv.) and 3-(1-methyl-7-piperazin-1-yl-indazol-3-yl)piperidine-2,6-dione (51.39 mg, 156.96 μmol, 1.1 equiv.) in DMF (2 mL) was added HATU (81.38 mg, 214.04 μmol, 1.5 equiv.) and DIEA (92.21 mg, 713.46 μmol, 124.27 μL, 5.0 equiv.). The mixture was stirred at 25° C. for 16 hours. The mixture was added into H₂O (5.0 ml) and filtered to give tert-butyl 6-[8-(1,3-benzothiazol-2-ylcarbamoyl)-3,4-dihydro-1H-isoquinolin-2-yl]-3-[3-[3-[(3R,5S)-4-[2-[4-[3-(2,6-dioxo-3-piperidyl)-1-methyl-indazol-7-yl]piperazin-1-yl]-2-oxo-ethyl]-3,5-dimethyl-piperazin-1-yl]-2,2-difluoro-propoxy]-2-methyl-phenyl]pyridine-2-carboxylate (145 mg, 126.0 μmol, 88.3% yield) was obtained as a brown solid.

MS (ESI) m/z: 1150.7 [M+H]⁺.

### Step B. Procedure for Preparation of 6-[8-(1,3-benzothiazol-2-ylcarbamoyl)-3,4-dihydro-1H-isoquinolin-2-yl]-3-[3-[3-[(3R,5S)-4-[2-[4-[3-(2,6-dioxo-3-piperidyl)-1-methyl-indazol-7-yl]piperazin-1-yl]-2-oxo-ethyl]-3,5-dimethyl-piperazin-1-yl]-2,2-difluoro-propoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[3-[(3R,5S)-4-[2-[4-[3-(2,6-dioxo-3-piperidyl)-1-methyl-indazol-7-yl]piperazin-1-yl]-2-oxo-ethyl]-3, 5-dimethyl-piperazin-1-yl]-2,2-difluoro-propoxy]-2-methyl-phenyl]pyridine-2-carboxylate (145 mg, 126.05 μmol, 1.0 equiv.) in DCM (1 mL) and TFA (1 mL). The mixture was stirred at 25° C. for 16 hours. The mixture was concentrated to remove DCM. The residue was purified by prep-HPLC to give 6-[8-(1,3-benzothiazol-2-ylcarbamoyl)-3,4-dihydro-1H-isoquinolin-2-yl]-3-[3-[3-[(3R,5S)-4-[2-[4-[3-(2,6-dioxo-3-piperidyl)-1-methyl-indazol-7-yl]piperazin-1-yl]-2-oxo-ethyl]-3,5-dimethyl-piperazin-1-yl]-2,2-difluoro-propoxy]-2-methyl-phenyl]pyridine-2-carboxylic acid (49.2 mg, 43.4 μmol, 34.4% yield, 96.6% purity) as a white solid.

MS (ESI) m/z: 548.0 [M12+H]⁺.

¹H NMR (400 MHz, DMSO-d₆) δ=12.89 (d, J=9.6 Hz, 1H), 10.88 (s, 1H), 8.13 (s, 1H), 8.03 (d, J=7.6 Hz, 1H), 7.78 (d, J=8.0 Hz, 1H), 7.62 (d, J=7.6 Hz, 1H), 7.49-7.31 (m, 6H), 7.17-7.10 (m, 1H), 7.05-6.95 (m, 4H), 6.71 (d, J=8.0 Hz, 1H), 4.98 (s, 2H), 4.38-4.29 (m, 3H), 4.26 (s, 3H), 3.92 (t, J=5.6 Hz, 2H), 3.10 (s, 4H), 3.03 (t, J=5.6 Hz, 4H), 2.86 (t, J=13.6 Hz, 4H), 2.75-2.57 (m, 7H), 2.33 (s, 2H), 2.16 (dd, J=5.2, 12.8 Hz, 1H), 2.07 (s, 2H), 1.92 (s, 3H), 0.92 (d, J=4.8 Hz, 6H).

¹⁹F NMR (400 MHz, DMSO-d₆) δ=106.40.

### Example 150. Preparation of Compound 182a

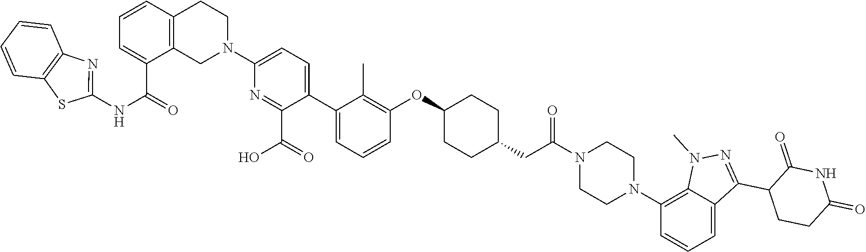 [SMILES: Cc1c(O[C@H]2CC[C@H](CC(=O)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-(2-(4-(3-(2,6-dioxopiperidin-3-yl)-1-methyl-1H-indazol-7-yl)piperazin-1-yl)-2-oxoethyl)cyclohexyl)oxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(((1r,4r)-4-(2-(4-(3-(2,6-dioxopiperidin-3-yl)-1-methyl-1H-indazol-7-yl)piperazin-1-yl)-2-oxoethyl)cyclohexyl)oxy)-2-methylphenyl)picolinate

To a solution of 2-((1r,4r)-4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)cyclohexyl)acetic acid (30 mg, 40.93 μmol, 1.0 equiv.) in pyridine (1 mL) was added EDCI (10.20 mg, 53.21 μmol, 1.3 equiv.) and 3-(1-methyl-7-(piperazin-1-yl)-1H-indazol-3-yl)piperidine-2,6-dione (13.40 mg, 40.93 μmol, 1.0 equiv.). The mixture was stirred at 60° C. for 12 hours. The reaction mixture was concentrated under reduced pressure to remove solvent. The residue was purified by prep-TLC (SiO₂, petroleum ether/ethyl acetate=0/1) to give tert-butyl 6-(8-(benzo[d]thiazol-2-ylcarbamoyl)-3,4-dihydroisoquinolin-2(1H)-yl)-3-(3-(((1r,4r)-4-(2-(4-(3-(2,6-dioxopiperidin-3-yl)-1-methyl-1H-indazol-7-yl)piperazin-1-yl)-2-oxoethyl)cyclohexyl)oxy)-2-methylphenyl)picolinate (30 mg, 28.8 μmol, 70.3% yield) as a yellow oil.

MS (ESI) m/z: 1042.5 [M+H]⁺.

### Step B. Procedure for Preparation of 6-(8-(benzo[d]thiazol-2-ylcarbamoyl)-3,4-dihydroisoquinolin-2(1H)-yl)-3-(3-(((1r,4r)-4-(2-(4-(3-(2,6-dioxopiperidin-3-yl)-1-methyl-1H-indazol-7-yl)piperazin-1-yl)-2-oxoethyl)cyclohexyl)oxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(((1r,4r)-4-(2-(4-(3-(2,6-dioxopiperidin-3-yl)-1-methyl-1H-indazol-7-yl)piperazin-1-yl)-2-oxoethyl)cyclohexyl)oxy)-2-methylphenyl)picolinate (50 mg, 47.97 μmol, 1.0 equiv.) in DCM (1 mL) was added TFA (5.47 mg, 47.97 μmol, 3.55 μL, 1.0 equiv.). The mixture was stirred at 40° C. for 4 hours. The reaction mixture was concentrated under reduced pressure to remove solvent. The residue was purified by prep-HPLC to give 6-(8-(benzo[d]thiazol-2-ylcarbamoyl)-3,4-dihydroisoquinolin-2(1H)-yl)-3-(3-(((1r,4r)-4-(2-(4-(3-(2,6-dioxopiperidin-3-yl)-1-methyl-1H-indazol-7-yl)piperazin-1-yl)-2-oxoethyl)cyclohexyl)oxy)-2-methylphenyl)picolinic acid (16.7 mg, 15.8 μmol, 33.0% yield, 93.5% purity) as a yellow solid.

MS (ESI) m/z: 986.5 [M+H]⁺.

¹H NMR (400 MHz, DMSO-d₆) δ=13.10-12.26 (m, 2H), 10.89 (s, 1H), 8.04 (d, J=8.0 Hz, 1H), 7.84-7.75 (m, 1H), 7.63 (d, J=7.6 Hz, 1H), 7.52-7.41 (m, 4H), 7.40-7.32 (m, 2H), 7.12-7.00 (m, 3H), 6.95 (dd, J=8.4, 14.8 Hz, 2H), 6.62 (d, J=7.2 Hz, 1H), 4.98 (s, 2H), 4.35 (dd, J=5.2, 9.6 Hz, 1H), 4.27 (s, 3H), 4.20 (t, J=9.2 Hz, 1H), 3.92 (s, 2H), 3.29-3.14 (m, 4H), 3.04 (d, J=5.2 Hz, 2H), 2.94-2.72 (m, 2H), 2.71-2.52 (m, 6H), 2.32 (d, J=4.4 Hz, 2H), 2.17 (dd, J=5.6, 13.2 Hz, 1H), 2.12-2.03 (m, 2H), 1.88 (s, 3H), 1.83 (d, J=12.0 Hz, 2H), 1.45-1.33 (m, 2H), 1.22-1.11 (m, 2H)

### Example 151. Preparation of Compound 183a

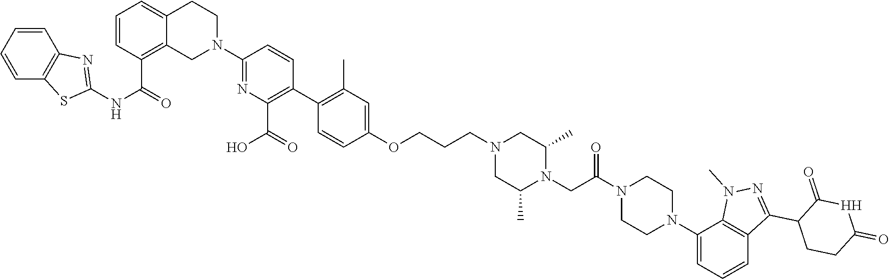

### 6-[8-(1,3-benzothiazol-2-ylcarbamoyl)-3,4-dihydro-1H-isoquinolin-2-yl]-3-[4-[3-[(3S,5R)-4-[2-[4-[3-(2,6-dioxo-3-piperidyl)-1-methyl-indazol-7-yl]piperazin-1-yl]-2-oxo-ethyl]-3, 5-dimethyl-piperazin-1-yl]propoxy]-2-methyl-phenyl]pyridine-2-carboxylic acid

### Step A. Procedure for Preparation of (3R,5S)-tert-butyl 4-(2-methoxy-2-oxoethyl)-3,5-dimethylpiperazine-1-carboxylate

A mixture of methyl 2-bromoacetate (9.35 g, 56.00 mmol, 6.19 mL, 1.5 equiv.), (3R, 5S)-tert-butyl 3,5-dimethylpiperazine-1-carboxylate (8 g, 37.33 mmol, 1 equiv.), and K₂CO₃ (15.48 g, 111.99 mmol, 3 equiv.) in CH₃CN (100 mL) was degassed and purged with N₂ and stirred at 60° C. for 3 hours under N₂ atmosphere. The reaction mixture was diluted with water (100 mL) and extracted with ethyl acetate (120 mL×3). The combined organic layers were washed with brine (120 mL×3), dried over anhydrous sodium sulfate, filtered, and concentrated under reduced pressure to give a residue. The residue was purified by flash silica gel chromatography (Eluent of 0˜25% ethyl acetate/petroleum ether) to give (3R,5S)-tert-butyl 4-(2-methoxy-2-oxoethyl)-3,5-dimethylpiperazine-1-carboxylate (10 g, 33.2 mmol, 89.1% yield) as a yellow oil.

¹H NMR (400 MHz, DMSO-d₆) δ=4.10-4.05 (m, 2H), 3.67 (s, 2H), 3.50 (s, 2H), 2.78-2.69 (m, 2H), 2.49-2.32 (m, 2H), 1.39 (s, 9H), 1.20-1.16 (m, 3H), 0.97 (d, J=6.4 Hz, 6H)

### Step B. Procedure for Preparation of methyl 2-((2R,6S)-2,6-dimethylpiperazin-1-yl)acetate

A mixture of (3R,5S)-tert-butyl 4-(2-methoxy-2-oxoethyl)-3,5-dimethylpiperazine-1-carboxylate (10 g, 33.29 mmol, 1 equiv.) and HCl/1,4-dioxane (4 M, 83.22 mL, 10 equiv.) in DCM (80 mL) was degassed and purged with N₂ three times, and then the mixture was stirred at 25° C. for 16 hours under N₂ atmosphere. The reaction mixture was concentrated under reduced pressure to give methyl 2-((2R,6S)-2,6-dimethylpiperazin-1-yl)acetate (12.25 g, crude, HCl) as a yellow solid.

¹H NMR (400 MHz, CD₃OD) δ=4.44 (s, 2H), 4.36 (q, J=7.2 Hz, 2H), 4.29-4.21 (m, 2H), 3.74-3.67 (m, 2H), 3.58-3.50 (m, 2H), 1.49 (d, J=6.4 Hz, 6H), 1.35 (t, J=7.2 Hz, 3H)

### Step C. Procedure for Preparation of 1-bromo-4-(3-bromopropoxy)-2-methylbenzene

A mixture of 4-bromo-3-methylphenol (3 g, 16.04 mmol, 1 equiv.), 1,3-dibromopropane (16.19 g, 80.20 mmol, 8.18 mL, 5 equiv.) and K₂CO₃ (6.65 g, 48.12 mmol, 3 equiv.) in CH₃CN (30 mL) was degassed and purged with N₂ and stirred at 70° C. for 12 hours under N₂ atmosphere. The reaction mixture was concentrated under reduced pressure to give a residue. The residue was purified by flash silica gel chromatography (Eluent of 0˜5% ethyl acetate/petroleum ether) to give compound 1-bromo-4-(3-bromopropoxy)-2-methylbenzene (2.5 g, 8.1 mmol, 50.6% yield) as a colorless oil.

¹H NMR (400 MHz, DMSO-d₆) δ=7.44 (d, J=8.8 Hz, 1H), 6.98 (d, J=2.8 Hz, 1H), 6.74 (dd, J=8.8, 2.8 Hz, 1H), 4.08-4.02 (m, 2H), 3.65 (t, J=6.4 Hz, 2H), 2.30 (s, 3H), 2.26-2.19 (m, 2H)

### Step D. Procedure for Preparation of ethyl 2-((2R,6S)-4-(3-(4-bromo-3-methylphenoxy)propyl)-2,6-dimethylpiperazin-1-yl)acetate

A mixture of 1-bromo-4-(3-bromopropoxy)-2-methylbenzene (2.34 g, 7.60 mmol, 1.2 equiv.), methyl 2-((2R,6S)-2,6-dimethylpiperazin-1-yl)acetate (1.5 g, 6.34 mmol, 1 equiv., HCl) and DIEA (4.09 g, 31.68 mmol, 5.52 mL, 5 equiv.) in DMF (40 mL) was degassed and purged with N₂ and stirred at 60° C. for 16 hours under N₂ atmosphere. The reaction mixture was diluted with water (100 mL) and extracted with DCM (100 mL×3). The combined organic layers were washed with brine (100 mL×6), dried over anhydrous sodium sulfate, filtered, and concentrated under reduced pressure to give a residue. The residue was purified by flash silica gel chromatography (Eluent of 0˜7% DCM/MeOH) to give compound ethyl 2-((2R,6S)-4-(3-(4-bromo-3-methylphenoxy)propyl)-2,6-dimethylpiperazin-1-yl)acetate (1.3 g, 3.0 mmol, 48.0% yield) as an orange oil.

¹H NMR (400 MHz, CD₃OD) δ=7.37 (d, J=8.8 Hz, 1H), 6.86 (d, J=3.0 Hz, 1H), 6.66 (dd, J=8.8, 3.2 Hz, 1H), 4.16 (q, J=7.2 Hz, 2H), 3.98 (t, J=6.0 Hz, 2H), 3.55 (s, 2H), 3.04-2.98 (m, 2H), 2.86-2.84 (m, 1H), 2.82 (s, 1H), 2.54-2.46 (m, 2H), 2.33 (s, 3H), 2.00-1.92 (m, 2H), 1.92-1.85 (m, 2H), 1.27 (t, J=7.2 Hz, 3H), 1.08 (d, J=6.4 Hz, 6H)

### Step E. Procedure for Preparation of tert-butyl 6-(8-(benzo[d]thiazol-2-ylcarbamoyl)-3,4-dihydroisoquinolin-2(1H)-yl)-3-(4-(3-((3R,5S)-4-(2-ethoxy-2-oxoethyl)-3,5-dimethylpiperazin-1-yl)propoxy)-2-methylphenyl)picolinate

Ethyl 2-((2R,6S)-4-(3-(4-bromo-3-methylphenoxy)propyl)-2,6-dimethylpiperazin-1-yl)acetate (400 mg, 935.95 μmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (687.97 mg, 1.12 mmol, 1.2 equiv.), Ad2nBuP Pd G₃ (cataCXium® A Pd G₃) (68.16 mg, 93.59 μmol, 0.1 equiv.) and KF (1.5 M, 1.87 mL, 3 equiv.) were taken up into a microwave tube in 1,4-dioxane (11 mL). The sealed tube was heated at 100° C. for 60 minutes under microwave. The reaction mixture was concentrated under reduced pressure to give a residue. The residue was purified by reverse-phase HPLC (with 0.1% HCOOH) to give tert-butyl 6-(8-(benzo[d]thiazol-2-ylcarbamoyl)-3,4-dihydroisoquinolin-2(1H)-yl)-3-(4-(3-((3R,5S)-4-(2-ethoxy-2-oxoethyl)-3,5-dimethylpiperazin-1-yl)propoxy)-2-methylphenyl)picolinate (410 mg, 477.4 μmol, 51.0% yield, 97.0% purity) as a yellow solid.

MS (ESI) m/z: 833.4 [M+H]⁺

¹H NMR (400 MHz, DMSO-d₆) δ=13.12-12.55 (m, 1H), 8.02 (d, J=8.0 Hz, 1H), 7.77 (d, J=8.0 Hz, 1H), 7.60 (d, J=7.6 Hz, 1H), 7.48-7.41 (m, 3H), 7.39-7.31 (m, 2H), 6.92 (d, J=8.8 Hz, 1H), 6.85 (d, J=8.4 Hz, 1H), 6.81 (s, 1H), 6.75-6.69 (m, 1H), 4.96 (s, 2H), 4.06 (q, J=7.2 Hz, 2H), 3.98 (t, J=6.0 Hz, 2H), 3.86 (t, J=6.0 Hz, 2H), 3.45 (s, 2H), 3.03 (t, J=5.6 Hz, 2H), 2.87-2.80 (m, 2H), 2.72 (d, J=10.4 Hz, 2H), 2.34 (t, J=6.8 Hz, 2H), 2.00 (s, 3H), 1.87-1.78 (m, 2H), 1.66 (t, J=10.4 Hz, 2H), 1.17 (t, J=7.2 Hz, 3H), 1.03 (s, 9H), 0.95 (d, J=6.4 Hz, 6H)

### Step F. Procedure for Preparation of 2-((2R,6S)-4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)propyl)-2,6-dimethylpiperazin-1-yl)acetic acid

A mixture of tert-butyl 6-(8-(benzo[d]thiazol-2-ylcarbamoyl)-3,4-dihydroisoquinolin-2(1H)-yl)-3-(4-(3-((3R,5S)-4-(2-ethoxy-2-oxoethyl)-3, 5-dimethylpiperazin-1-yl)propoxy)-2-methylphenyl)picolinate (400 mg, 480.17 μmol, 1 equiv.) and LiOH·H₂O (100.75 mg, 2.40 mmol, 5 equiv.) in THE (4 mL) and H₂O (1 mL) was degassed and purged with N₂ and stirred at 40° C. for 7 hours under N₂ atmosphere. The reaction mixture was concentrated under reduced pressure to give 2-((2R,6S)-4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)propyl)-2,6-dimethylpiperazin-1-yl)acetic acid (400 mg, crude) as a yellow solid.

MS (ESI) m/z: 805.7 [M+H]⁺

### Step G. Procedure for Preparation of tert-butyl 6-[8-(1,3-benzothiazol-2-ylcarbamoyl)-3,4-dihydro-1H-isoquinolin-2-yl]-3-[4-[3-[(3S,5R)-4-[2-[4-[3-(2,6-dioxo-3-piperidyl)-1-methyl-indazol-7-yl]piperazin-1-yl]-2-oxo-ethyl]-3,5-dimethyl-piperazin-1-yl]propoxy]-2-methyl-phenyl]pyridine-2-carboxylate

To a solution of 2-((2R,6S)-4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)propyl)-2,6-dimethylpiperazin-1-yl)acetic acid (80 mg, 99.38 μmol, 1 equiv.), 3-(1-methyl-7-piperazin-1-yl-indazol-3-yl)piperidine-2,6-dione (39.04 mg, 119.26 μmol, 1.2 equiv.) in DMF (1 mL) was added DIEA (51.38 mg, 397.52 μmol, 69.24 μL, 4 equiv.) and HATU (56.68 mg, 149.07 μmol, 1.5 equiv.). The mixture was stirred at 25° C. for 12 hours. The reaction mixture was quenched by water (2 mL) at 25° C. and precipitated in water to give compound tert-butyl 6-[8-(1,3-benzothiazol-2-ylcarbamoyl)-3,4-dihydro-1H-isoquinolin-2-yl]-3-[4-[3-[(3S,5R)-4-[2-[4-[3-(2,6-dioxo-3-piperidyl)-1-methyl-indazol-7-yl]piperazin-1-yl]-2-oxo-ethyl]-3,5-dimethyl-piperazin-1-yl]propoxy]-2-methyl-phenyl]pyridine-2-carboxylate (180 mg, crude) as a white solid.

MS (ESI) m/z: 1114.9[M+H]⁺

### Step H. Procedure for Preparation of 6-[8-(1,3-benzothiazol-2-ylcarbamoyl)-3,4-dihydro-1H-isoquinolin-2-yl]-3-[4-[3-[(3S,5R)-4-[2-[4-[3-(2,6-dioxo-3-piperidyl)-1-methyl-indazol-7-yl]piperazin-1-yl]-2-oxo-ethyl]-3,5-dimethyl-piperazin-1-yl]propoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[4-[3-[(3S,5R)-4-[2-[4-[3-(2,6-dioxo-3-piperidyl)-1-methyl-indazol-7-yl]piperazin-1-yl]-2-oxo-ethyl]-3,5-dimethyl-piperazin-1-yl]propoxy]-2-methyl-phenyl]pyridine-2-carboxylate (180 mg, 161.53 μmol, 1 equiv.) in DCM (2 mL) was added TFA (18.42 mg, 161.53 μmol, 11.96 μL, 1 equiv.). The mixture was stirred at 25° C. for 12 hours. The reaction mixture concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[4-[3-[(3S,5R)-4-[2-[4-[3-(2,6-dioxo-3-piperidyl)-1-methyl-indazol-7-yl]piperazin-1-yl]-2-oxo-ethyl]-3,5-dimethyl-piperazin-1-yl]propoxy]-2-methyl-phenyl]pyridine-2-carboxylic acid as a white solid.

MS (ESI) m/z: 1058.8 [M+H]⁺.

¹HNMR (400 MHz, DMSO-d₆) δ=13.22-12.27 (m, 2H), 10.90 (s, 1H), 8.13 (s, 1H), 8.03 (d, J=7.6 Hz, 1H), 7.79 (d, J=7.6 Hz, 1H), 7.62 (d, J=8.0 Hz, 1H), 7.49-7.42 (m, 4H), 7.40-7.32 (m, 2H), 7.06-7.02 (m, 2H), 6.94 (dd, J=8.8, 10.8 Hz, 2H), 6.79 (d, J=2.0 Hz, 1H), 6.72 (dd, J=2.4, 8.4 Hz, 1H), 4.97 (s, 2H), 4.35 (dd, J=5.2, 9.6 Hz, 1H), 4.27 (s, 3H), 4.03-3.99 (m, 2H), 3.91 (t, J=6.0 Hz, 2H), 3.78-3.57 (m, 2H), 3.29-3.15 (m, 8H), 3.02 (t, J=5.2 Hz, 4H), 2.71-2.60 (m, 4H), 2.37-2.30 (m, 2H), 2.19-2.13 (m, 2H), 2.09-1.84 (m, 7H), 1.02 (s, 6H).

### Example 152. Preparation of Compound 185a

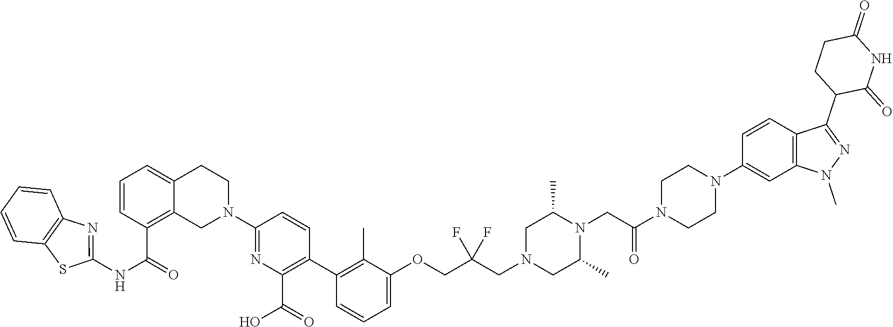 [SMILES: Cc1c(OCC(F)(F)CN2C[C@@H](C)N(CC(=O)N3CCN(c4ccc5c(C6CCC(=O)NC6=O)nn(C)c5c4)CC3)[C@@H](C)C2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[(3R,5S)-4-[2-[4-[3-(2,6-dioxo-3-piperidyl)-1-methyl-indazol-6-yl]piperazin-1-yl]-2-oxo-ethyl]-3, 5-dimethyl-piperazin-1-yl]-2,2-difluoro-propoxy]-2-methyl-phenyl]pyridine-2-carboxylic acid

### Step A. Procedure for Preparation of tert-butyl 6-[8-(1,3-benzothiazol-2-ylcarbamoyl)-3,4-dihydro-1H-isoquinolin-2-yl]-3-[3-[3-[(3R,5S)-4-[2-[4-[3-(2,6-dioxo-3-piperidyl)-1-methyl-indazol-6-yl]piperazin-1-yl]-2-oxo-ethyl]-3,5-dimethyl-piperazin-1-yl]-2,2-difluoro-propoxy]-2-methyl-phenyl]pyridine-2-carboxylate

To a solution of 2-[(2R, 6S)-4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-2,2-difluoro-propyl]-2,6-dimethyl-piperazin-1-yl]acetic acid (100 mg, 118.91 μmol, 1.0 equiv.) and 3-(1-methyl-6-piperazin-1-yl-indazol-3-yl)piperidine-2,6-dione (58.39 mg, 178.36 μmol, 1.5 equiv.) in DMF (3 mL) was added HATU (54.26 mg, 142.69 μmol, 1.2 equiv.) and DIEA (46.10 mg, 356.73 μmol, 62.14 μL, 3.0 equiv.). The mixture was stirred at 25° C. for 2 hours. The reaction mixture was added into H₂O (5 mL). The mixture was triturated with water (5 mL) at 25° C. for 30 minutes and filtered to give tert-butyl 6-[8-(1,3-benzothiazol-2-ylcarbamoyl)-3,4-dihydro-1H-isoquinolin-2-yl]-3-[3-[3-[(3R,5S)-4-[2-[4-[3-(2,6-dioxo-3-piperidyl)-1-methyl-indazol-6-yl]piperazin-1-yl]-2-oxo-ethyl]-3,5-dimethyl-piperazin-1-yl]-2,2-difluoro-propoxy]-2-methyl-phenyl]pyridine-2-carboxylate (135 mg, 117.4 μmol, 98.7% yield) as a yellow solid.

MS (ESI) m/z: 1150.6 [M+H]⁺.

### Step B. Procedure for Preparation of 6-[8-(1,3-benzothiazol-2-ylcarbamoyl)-3,4-dihydro-1H-isoquinolin-2-yl]-3-[3-[3-[(3R,5S)-4-[2-[4-[3-(2,6-dioxo-3-piperidyl)-1-methyl-indazol-6-yl]piperazin-1-yl]-2-oxo-ethyl]-3,5-dimethyl-piperazin-1-yl]-2,2-difluoro-propoxy]-2-methyl-phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[3-[(3R,5S)-4-[2-[4-[3-(2,6-dioxo-3-piperidyl)-1-methyl-indazol-6-yl]piperazin-1-yl]-2-oxo-ethyl]-3,5-dimethyl-piperazin-1-yl]-2,2-difluoro-propoxy]-2-methyl-phenyl]pyridine-2-carboxylate (135 mg, 117.36 μmol, 1.0 equiv.) in DCM (1 mL) and TFA (1 mL) was stirred at 25° C. for 16 hours. The mixture was concentrated to remove DCM. The residue was purified by prep-HPLC to give 6-[8-(1,3-benzothiazol-2-ylcarbamoyl)-3,4-dihydro-1H-isoquinolin-2-yl]-3-[3-[3-[(3R,5S)-4-[2-[4-[3-(2,6-dioxo-3-piperidyl)-1-methyl-indazol-6-yl]piperazin-1-yl]-2-oxo-ethyl]-3,5-dimethyl-piperazin-1-yl]-2,2-difluoro-propoxy]-2-methyl-phenyl]pyridine-2-carboxylic acid (68.0 mg, 59.6 μmol, 50.8% yield, 95.8% purity) as a white solid. MS (ESI) m/z: 1094.4 [M+H]⁺.

¹H NMR (400 MHz, DMSO-d₆) δ=12.90-12.43 (m, 2H), 10.85 (s, 1H), 8.13 (s, 1H), 8.02 (d, J=8.0 Hz, 1H), 7.78 (d, J=8.0 Hz, 1H), 7.62 (d, J=7.2 Hz, 1H), 7.53 (d, J=8.8 Hz, 1H), 7.50-7.43 (m, 3H), 7.41-7.302 (m, 2H), 7.18-7.13 (m, 1H), 7.04-6.85 (m, 4H), 6.71 (d, J=7.6 Hz, 1H), 4.99 (s, 2H), 4.41-4.22 (m, 3H), 3.97-3.85 (m, 5H), 3.68-3.50 (m, 6H), 3.22 (d, J=2.4 Hz, 4H), 3.03 (t, J=5.6 Hz, 2H), 2.93-2.80 (m, 2H), 2.68-2.50 (m, 6H), 2.37-2.10 (m, 3H), 2.09-1.98 (m, 1H), 1.92 (s, 3H), 1.17-0.78 (m, 6H)

¹⁹F NMR (400 MHz, DMSO-d₆) δ=106.40.

### Example 153. Preparation of Compound 186a

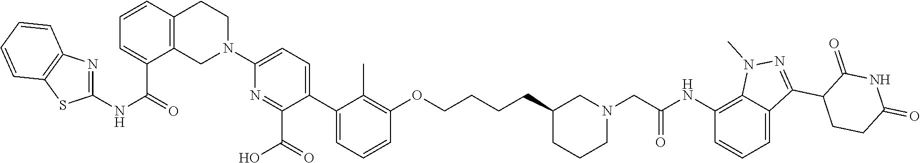 [SMILES: Cc1c(OCCCC[C@@H]2CCCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[4-[(3R)-1-[2-[[3-(2,6-dioxo-3-piperidyl)-1-methyl-indazol-7-yl]amino]-2-oxo-ethyl]-3-piperidyl]butoxy]-2-methyl-phenyl]pyridine-2-carboxylic acid

### Step A. Procedure for Preparation of tert-butyl (3R)-3-[4-(3-bromo-2-methyl-phenoxy)butyl]piperidine-1-carboxylate

A mixture of tert-butyl (3R)-3-(4-hydroxybutyl)piperidine-1-carboxylate (1.3 g, 5.05 mmol, 1 equiv.), 3-bromo-2-methyl-phenol (755.79 mg, 4.04 mmol, 0.8 equiv.), and 2-(tributyl-λ5-phosphanylidene)acetonitrile (1.46 g, 6.06 mmol, 1.2 equiv.) in toluene (30 mL) was stirred at 120° C. for 2 hours under N₂ atmosphere. The reaction mixture was filtered and concentrated under reduced pressure to give a residue. The residue was purified by flash silica gel chromatography (Eluent of 10% ethyl acetate/petroleum ether). Then the residue was further separated by SFC to give tert-butyl (3R)-3-[4-(3-bromo-2-methyl-phenoxy)butyl]piperidine-1-carboxylate (1.1 g, 2.5 mmol, 50.7% yield, 99.3% purity) as a yellow oil.

MS (ESI) m/z: 328.0 [M−100+H]⁺.

¹H NMR (400 MHz, DMSO-d₆) δ=7.17-7.13 (m, 1H), 7.12-7.06 (m, 1H), 6.97 (d, J=8.0 Hz, 1H), 3.98 (t, J=6.4 Hz, 2H), 3.85-3.68 (m, 2H), 2.80-2.73 (m, 1H), 2.49-2.26 (m, 1H), 2.23 (s, 3H), 1.78-1.69 (m, 3H), 1.60-1.53 (m, 1H), 1.49-1.43 (m, 2H), 1.38 (s, 9H), 1.36-1.27 (m, 2H), 1.27-1.20 (m, 2H), 1.14-1.01 (m, 1H).

### Step B. Procedure for Preparation of (3R)-3-[4-(3-bromo-2-methyl-phenoxy)butyl]piperidine

A mixture of tert-butyl (3R)-3-[4-(3-bromo-2-methyl-phenoxy)butyl]piperidine-1-carboxylate (400 mg, 938.12 μmol, 1 equiv.) in DCM (2.5 mL) and HCl/dioxane (12 mL) was stirred at 25° C. for 16 hours. The reaction mixture was concentrated under reduced pressure to give (3R)-3-[4-(3-bromo-2-methyl-phenoxy)butyl]piperidine (349 mg, crude) as a white solid.

MS (ESI) m/z: 328.1 [M+H]⁺.

¹H NMR (400 MHz, DMSO-d₆) δ=7.17-7.13 (m, 1H), 7.12-7.06 (m, 1H), 6.96 (d, J=8.0 Hz, 1H), 3.98 (t, J=6.4 Hz, 2H), 3.18 (d, J=12.0 Hz, 2H), 2.78-2.67 (m, 1H), 2.48-2.45 (m, 1H), 2.23 (s, 3H), 1.83-1.52 (m, 7H), 1.48-1.39 (m, 2H), 1.32-1.22 (m, 2H), 1.16-1.04 (m, 1H).

### Step C. Procedure for Preparation of ethyl 2-[(3R)-3-[4-(3-bromo-2-methyl-phenoxy)butyl]-1-piperidyl]acetate

A mixture of (3R)-3-[4-(3-bromo-2-methyl-phenoxy)butyl]piperidine (349 mg, 1.07 mmol, 1 equiv.), ethyl 2-bromoacetate (142.91 mg, 855.73 μmol, 94.64 μL, 0.8 equiv.), and K₂CO₃ (443.50 mg, 3.21 mmol, 3 equiv.) in DMF (10 mL) was stirred at 60° C. for 2 hours under N₂ atmosphere. The reaction mixture was concentrated under reduced pressure to give a residue. The crude product was purified by reverse-phase HPLC (with 0.1% HCOOH) to give ethyl 2-[(3R)-3-[4-(3-bromo-2-methyl-phenoxy)butyl]-1-piperidyl]acetate (374 mg, 896.9 μmol, 83.8% yield, 98.9% purity) as a yellow oil.

MS (ESI) m/z: 414.0 [M+H]⁺.

¹H NMR (400 MHz, DMSO-d₆) δ=8.14 (s, 1H), 7.16-7.12 (m, 1H), 7.11-7.05 (m, 1H), 6.95 (d, J=7.6 Hz, 1H), 4.12 (q, J=7.2 Hz, 2H), 3.97 (t, J=6.4 Hz, 2H), 3.51 (s, 2H), 3.05-2.92 (m, 2H), 2.44-2.33 (m, 1H), 2.22 (s, 3H), 2.13 (t, J=10.8 Hz, 1H), 1.73-1.69 (m, 2H), 1.66-1.58 (m, 2H), 1.43 (td, J=7.6, 14.8 Hz, 2H), 1.33-1.09 (m, 6H), 0.84-0.95 (m, 1H).

### Step D. Procedure for Preparation of tert-butyl 6-[8-(1,3-benzothiazol-2-ylcarbamoyl)-3,4-dihydro-1H-isoquinolin-2-yl]-3-[3-[4-[(3R)-1-(2-ethoxy-2-oxo-ethyl)-3-piperidyl]butoxy]-2-methyl-phenyl]pyridine-2-carboxylate

Ethyl 2-[(3R)-3-[4-(3-bromo-2-methyl-phenoxy)butyl]-1-piperidyl]acetate (421 mg, 1.02 mmol, 1.05 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (596.69 mg, 974.12 μmol, 1 equiv.), Ad₂nBuP Pd G₃ (cataCXium® A Pd G₃) (70.94 mg, 97.41 μmol, 0.1 equiv.), and KF (1.5 M, 1.95 mL, 3 equiv.) were taken up into a microwave tube in 1,4-dioxane (10 mL). The sealed tube was heated at 100° C. for 60 minutes under microwave. The reaction mixture was filtered and concentrated under reduced pressure to give a residue. The residue was purified by prep-HPLC to give tert-butyl 6-[8-(1,3-benzothiazol-2-ylcarbamoyl)-3,4-dihydro-1H-isoquinolin-2-yl]-3-[3-[4-[(3R)-1-(2-ethoxy-2-oxo-ethyl)-3-piperidyl]butoxy]-2-methyl-phenyl]pyridine-2-carboxylate (511 mg, 593.5 μmol, 60.9% yield, 95.0% purity) as a yellow oil.

MS (ESI) m/z: 818.4 [M+H]⁺.

¹H NMR (400 MHz, CDCl₃) δ=7.80 (d, J=7.6 Hz, 1H), 7.59 (dd, J=7.6, 15.2 Hz, 2H), 7.41-7.35 (m, 1H), 7.34-7.29 (m, 3H), 7.27 (d, J=7.6 Hz, 1H), 7.03 (t, J=8.0 Hz, 1H), 6.82 (d, J=8.8 Hz, 1H), 6.73 (d, J=8.0 Hz, 1H), 6.64 (d, J=7.6 Hz, 1H), 5.24 (s, 1H), 5.05-4.91 (m, 2H), 4.18 (q, J=7.2 Hz, 2H), 4.03 (t, J=5.6 Hz, 2H), 3.90 (t, J=6.0 Hz, 3H), 3.81 (s, 2H), 3.01 (t, J=5.6 Hz, 3H), 2.04-1.95 (m, 2H), 1.91 (s, 5H), 1.76-1.71 (m, 2H), 1.54-1.38 (m, 3H), 1.27-1.22 (m, 4H), 1.20 (d, J=6.0 Hz, 3H), 1.08 (s, 9H).

### Step E. Procedure for Preparation of 2-[(3R)-3-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]butyl]-1-piperidyl]acetic acid

A mixture of tert-butyl 6-[8-(1,3-benzothiazol-2-ylcarbamoyl)-3,4-dihydro-1H-isoquinolin-2-yl]-3-[3-[4-[(3R)-1-(2-ethoxy-2-oxo-ethyl)-3-piperidyl]butoxy]-2-methyl-phenyl]pyridine-2-carboxylate (112 mg, 136.91 μmol, 1 equiv.) and LiOH·H₂O (17.24 mg, 410.74 μmol, 3 equiv.) in THE (2 mL) and H₂O (1 mL) was stirred at 25° C. for 16 hours. The reaction mixture was quenched by water (5 mL), and then extracted with ethyl acetate (10 mL×3). The organic layers was combined and concentrated under reduced pressure to give 2-[(3R)-3-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]butyl]-1-piperidyl]acetic acid (105 mg, crude) as a yellow oil.

MS (ESI) m/z: 790.4 [M+H]⁺.

### Step F. Procedure for Preparation of tert-butyl 6-[8-(1,3-benzothiazol-2-ylcarbamoyl)-3,4-dihydro-1H-isoquinolin-2-yl]-3-[3-[4-[(3R)-1-[2-[[3-(2,6-dioxo-3-piperidyl)-1-methyl-indazol-7-yl]amino]-2-oxo-ethyl]-3-piperidyl]butoxy]-2-methyl-phenyl]pyridine-2-carboxylate

A mixture of 2-[(3R)-3-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]butyl]-1-piperidyl]acetic acid (95 mg, 120.26 μmol, 1 equiv.), 3-(7-amino-1-methyl-indazol-3-yl)piperidine-2,6-dione (37.27 mg, 144.31 μmol, 1.2 equiv.), HATU (54.87 mg, 144.31 μmol, 1.2 equiv.), and DIEA (46.63 mg, 360.78 μmol, 62.84 μL, 3 equiv.) in DMF (4 mL) was stirred at 25° C. for 12 hours. The reaction mixture was quenched by water (5 mL) and then extracted with ethyl acetate (10 mL×3). The organic layers was combined and concentrated under reduced pressure to give tert-butyl 6-[8-(1,3-benzothiazol-2-ylcarbamoyl)-3,4-dihydro-1H-isoquinolin-2-yl]-3-[3-[4-[(3R)-1-[2-[[3-(2,6-dioxo-3-piperidyl)-1-methyl-indazol-7-yl]amino]-2-oxo-ethyl]-3-piperidyl]butoxy]-2-methyl-phenyl]pyridine-2-carboxylate (100 mg, crude) as a purple oil.

MS (ESI) m/z: 1030.7 [M+H]⁺.

### Step G. Procedure for Preparation of 6-[8-(1,3-benzothiazol-2-ylcarbamoyl)-3,4-dihydro-1H-isoquinolin-2-yl]-3-[3-[4-[(3R)-1-[2-[[3-(2,6-dioxo-3-piperidyl)-1-methyl-indazol-7-yl]amino]-2-oxo-ethyl]-3-piperidyl]butoxy]-2-methyl-phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[3-[4-[(3R)-1-[2-[[3-(2,6-dioxo-3-piperidyl)-1-methyl-indazol-7-yl]amino]-2-oxo-ethyl]-3-piperidyl]butoxy]-2-methyl-phenyl]pyridine-2-carboxylate (100 mg, 97.06 μmol, 1 equiv.) in TFA (1 mL) and DCM (1 mL) was stirred at 25° C. for 16 hours. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[4-[(3R)-1-[2-[[3-(2,6-dioxo-3-piperidyl)-1-methyl-indazol-7-yl]amino]-2-oxo-ethyl]-3-piperidyl]butoxy]-2-methyl-phenyl]pyridine-2-carboxylic acid (27.9 mg, 27.4 μmol, 28.2% yield, 95.8% purity) as a yellow solid.

MS (ESI) m/z: 974.6 [M+H]⁺.

¹H NMR (400 MHz, DMSO-d₆,) δ=10.89 (s, 1H), 9.83 (s, 1H), 8.13 (s, 1H), 8.02 (d, J=8.0 Hz, 1H), 7.78 (d, J=8.0 Hz, 1H), 7.61 (d, J=7.2 Hz, 1H), 7.55 (d, J=8.0 Hz, 1H), 7.50-7.33 (m, 5H), 7.29 (d, J=7.2 Hz, 1H), 7.00-7.10 (m, 2H), 6.95 (d, J=8.8 Hz, 1H), 6.86 (d, J=8.0 Hz, 1H), 6.61 (d, J=7.6 Hz, 1H), 4.97 (s, 2H), 4.35 (dd, J=5.2, 10.0 Hz, 1H), 4.10 (s, 3H), 3.80-4.00 (m, 4H), 3.16 (s, 2H), 3.02 (t, J=5.6 Hz, 2H), 2.95-2.84 (m, 2H), 2.67-2.57 (m, 2H), 2.38-2.28 (m, 1H), 2.21-2.07 (m, 2H), 1.87 (s, 4H), 1.77-1.40 (m, 9H), 1.32-1.20 (m, 2H).

### Example 154. Preparation of Compound 188

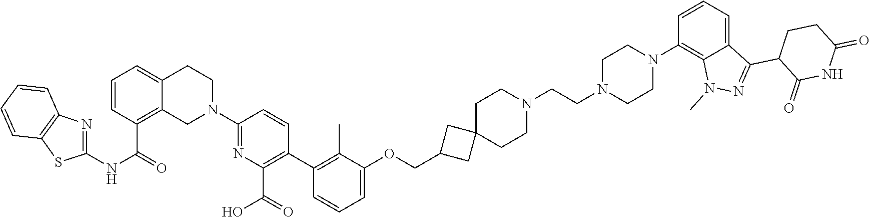 [SMILES: Cc1c(OCC2CC3(CCN(CCN4CCN(c5cccc6c(C7CCC(=O)NC7=O)nn(C)c56)CC4)CC3)C2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[[7-[2-[4-[3-(2,6-dioxo-3-piperidyl)-1-methyl-indazol-7-yl]piperazin-1-yl]ethyl]-7-azaspiro[3.5]nonan-2-yl]methoxy]-2-methyl-phenyl]pyridine-2-carboxylic acid

### Step A. Procedure for Preparation of tert-butyl 2-((3-bromo-2-methylphenoxy)methyl)-7-azaspiro[3.5]nonane-7-carboxylate

A mixture of tert-butyl 2-(hydroxymethyl)-7-azaspiro[3.5]nonane-7-carboxylate (1 g, 3.92 mmol, 1.2 equiv.), 3-bromo-2-methylphenol (610.38 mg, 3.26 mmol, 1 equiv.), and 2-(tributyl-λ⁵-phosphanylidene)acetonitrile (1.18 g, 4.90 mmol, 1.5 equiv.) in toluene (10 mL) was stirred at 120° C. for 2 h under N₂ atmosphere. The combined organic layers were filtered and concentrated under reduced pressure to give a residue. The residue was purified by flash silica gel chromatography (Eluent of 0˜8% ethyl acetate/petroleum ether) to give tert-butyl 2-((3-bromo-2-methylphenoxy)methyl)-7-azaspiro[3.5]nonane-7-carboxylate (1.3 g, 3.1 mmol, 96.7% yield) as a white solid.

¹H NMR (400 MHz, DMSO-d₆) δ=7.16 (d, J=8.0 Hz, 1H), 7.09 (t, J=8.0 Hz, 1H), 6.96 (d, J=8.0 Hz, 1H), 3.95 (d, J=6.2 Hz, 2H), 3.29-3.25 (m, 2H), 3.22-3.15 (m, 2H), 2.77-2.64 (m, 1H), 2.24 (s, 3H), 1.94-1.86 (m, 2H), 1.69-1.62 (m, 2H), 1.56-1.50 (m, 2H), 1.44-1.40 (m, 2H), 1.38 (s, 9H)

### Step B. Procedure for Preparation of 2-((3-bromo-2-methylphenoxy)methyl)-7-azaspiro[3.5]nonane

A mixture of tert-butyl 2-((3-bromo-2-methylphenoxy)methyl)-7-azaspiro[3.5]nonane-7-carboxylate (1.34 g, 3.16 mmol, 1 equiv.) in HCl/dioxane (15 mL) was stirred at 25° C. for 1 h, The combined organic layers were filtered and concentrated under reduced pressure to give a residue. The residue was used in the next step without other purification. The compound 2-((3-bromo-2-methylphenoxy)methyl)-7-azaspiro[3.5]nonane (1.32 g, crude, HCl) was obtained as a white solid.

¹H NMR (400 MHz, DMSO-d₆) δ=9.0-8.75 (m, 2H), 7.15 (d, J=7.2 Hz, 1H), 7.09 (t, J=8.0 Hz, 1H), 6.96 (d, J=8.0 Hz, 1H), 3.95 (d, J=6.2 Hz, 2H), 3.02-2.93 (m, 2H), 2.93-2.84 (m, 2H), 2.74-2.63 (m, 1H), 2.23 (s, 3H), 2.00-1.92 (m, 2H), 1.82-1.77 (m, 2H), 1.74-1.66 (m, 4H)

### Step C. Procedure for Preparation of 2-((3-bromo-2-methylphenoxy)methyl)-7-(2,2-diethoxyethyl)-7-azaspiro[3.5]nonane

A mixture of 2-((3-bromo-2-methylphenoxy)methyl)-7-azaspiro[3.5]nonane (1.22 g, 3.38 mmol, 1 equiv., HCl), 2-bromo-1,1-diethoxy-ethane (666.52 mg, 3.38 mmol, 508.80 μL, 1 equiv.), K₂CO₃ (2.34 g, 16.91 mmol, 5 equiv.), and KI (280.72 mg, 1.69 mmol, 0.5 equiv.) in CH₃CN (10 mL) was stirred at 80° C. for 16 hours. The combined organic layers were filtered and concentrated under reduced pressure to give a residue. The mixture was purified by flash silica gel chromatography (Eluent of 0˜100% ethyl acetate/petroleum ether) to give 2-((3-bromo-2-methylphenoxy)methyl)-7-(2,2-diethoxyethyl)-7-azaspiro[3.5]nonane (1.3 g, 2.9 mmol, 87.2% yield) as a yellow oil.

MS (ESI) m/z: 442.4[M+H]⁺.

### Step D. Procedure for Preparation of tert-butyl 6-(8-(benzo[d]thiazol-2-ylcarbamoyl)-3,4-dihydroisoquinolin-2(1H)-yl)-3-(3-((7-(2-ethoxy-2-methoxyethyl)-7-azaspiro[3.5]nonan-2-yl)methoxy)-2-methylphenyl)picolinate

A mixture of 2-((3-bromo-2-methylphenoxy)methyl)-7-(2,2-diethoxyethyl)-7-azaspiro[3.5]nonane (400 mg, 908.24 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (723.24 mg, 1.18 mmol, 1.3 equiv.), [2-(2-aminophenyl)phenyl]palladium(1⁺); bis(1-adamantyl)-butyl-phosphane; methanesulfonate (132.29 mg, 181.65 μmol, 0.2 equiv.), and K₂CO₃ (1.5 M, 1.98 mL, 3 equiv.) in dioxane (4 mL) was degassed and purged with N₂ and stirred at 100° C. for 1 h under N₂ atmosphere. The combined organic layers were filtered and concentrated under reduced pressure to give a residue. The mixture was purified by flash silica gel chromatography (Eluent of 50˜60% ethyl acetate/petroleum ether) to give tert-butyl 6-(8-(benzo[d]thiazol-2-ylcarbamoyl)-3,4-dihydroisoquinolin-2(1H)-yl)-3-(3-((7-(2-ethoxy-2-methoxyethyl)-7-azaspiro[3.5]nonan-2-yl)methoxy)-2-methylphenyl)picolinate (595 mg, 703.2 μmol, 77.4% yield) as a yellow oil.

MS (ESI) m/z: 846.7[M+H]⁺.

### Step E. Procedure for Preparation of 6-[8-(1,3-benzothiazol-2-ylcarbamoyl)-3,4-dihydro-1H-isoquinolin-2-yl]-3-[2-methyl-3-[[7-(2-oxoethyl)-7-azaspiro[3.5]nonan-2-yl]methoxy]phenyl]pyridine-2-carboxylic acid

A mixture of tert-butyl 6-(8-(benzo[d]thiazol-2-ylcarbamoyl)-3,4-dihydroisoquinolin-2(1H)-yl)-3-(3-((7-(2-ethoxy-2-methoxyethyl)-7-azaspiro[3.5]nonan-2-yl)methoxy)-2-methylphenyl)picolinate (245 mg, 289.57 μmol, 1 equiv.), in HCOOH (3 mL) was stirred at 90° C. for 1.5 hours. The combined organic layers were filtered and concentrated under reduced pressure to give a residue. The residue was used in the next step without other purification. The compound 6-(8-(benzo[d]thiazol-2-ylcarbamoyl)-3,4-dihydroisoquinolin-2(1H)-yl)-3-(2-methyl-3-((7-(2-oxoethyl)-7-azaspiro[3.5]nonan-2-yl)methoxy)phenyl)picolinic acid (200 mg, crude) was obtained as a yellow oil.

MS (ESI) m/z: 716.6[M+H]⁺.

### Step F. Procedure for Preparation of 6-[8-(1,3-benzothiazol-2-ylcarbamoyl)-3,4-dihydro-1H-isoquinolin-2-yl]-3-[3-[[7-[2-[4-[3-(2,6-dioxo-3-piperidyl)-1-methyl-indazol-7-yl]piperazin-1-yl]ethyl]-7-azaspiro[3.5]nonan-2-yl]methoxy]-2-methyl-phenyl]pyridine-2-carboxylic acid

A mixture of 3-(1-methyl-7-piperazin-1-yl-indazol-3-yl)piperidine-2,6-dione (27.44 mg, 83.82 μmol, 1.2 equiv.), NaBH(OAc)₃ (44.41 mg, 209.54 μmol, 3.0 equiv.), and NMM (7.06 mg, 69.85 μmol, 7.68 μL, 1.0 equiv.) in DCM (1 mL) and isopropanol (1 mL) was stirred at 0° C. for 5 min. To the mixture was then added 6-[8-(1,3-benzothiazol-2-ylcarbamoyl)-3,4-dihydro-1H-isoquinolin-2-yl]-3-[2-methyl-3-[[7-(2-oxoethyl)-7-azaspiro[3.5]nonan-2-yl]methoxy]phenyl]pyridine-2-carboxylic acid (50 mg, 69.85 μmol, 1.0 equiv.). The mixture was stirred at 0° C. for 30 min. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[[7-[2-[4-[3-(2,6-dioxo-3-piperidyl)-1-methyl-indazol-7-yl]piperazin-1-yl]ethyl]-7-azaspiro[3.5]nonan-2-yl]methoxy]-2-methyl-phenyl]pyridine-2-carboxylic acid (22.3 mg, 21.6 μmol, 15.5% yield, 99.5% purity) as a white solid.

MS (ESI) m/z: 1027.4[M+H]⁺

¹H NMR (400 MHz, DMSO-d₆) δ=10.87 (s, 1H), 8.05-7.97 (m, 1H), 7.78 (d, J=8.4 Hz, 1H), 7.65-7.59 (m, 1H), 7.48-7.31 (m, 6H), 7.10-6.98 (m, 3H), 6.94-6.83 (m, 2H), 6.64 (d, J=7.6 Hz, 1H), 4.97 (s, 2H), 4.33 (dd, J=4.8, 9.2 Hz, 1H), 4.23 (s, 3H), 3.94-3.87 (m, 4H), 3.58-3.50 (m, 4H), 3.04-2.97 (m, 4H), 2.93-2.77 (m, 4H), 2.70-2.59 (m, 4H), 2.41-2.26 (m, 6H), 2.19-2.14 (m, 1H), 1.93-1.84 (m, 5H), 1.68-1.56 (m, 4H), 1.52-1.45 (m, 2H)

### Example 155. Preparation of Compound 194

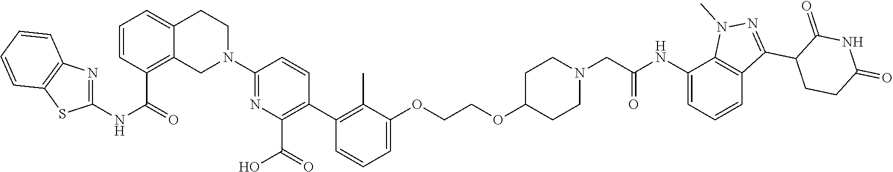 [SMILES: Cc1c(OCCOC2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[2-[[1-[2-[[3-(2,6-dioxo-3-piperidyl)-1-methyl-indazol-7-yl]amino]-2-oxo-ethyl]-4-piperidyl]oxy]ethoxy]-2-methyl-phenyl]pyridine-2-carboxylic acid

### Step A. Procedure for Preparation of tert-butyl 4-(2-(3-bromo-2-methylphenoxy)ethoxy)piperidine-1-carboxylate

A mixture of 3-bromo-2-methylphenol (655.68 mg, 3.51 mmol, 1 equiv.), tert-butyl 4-(2-hydroxyethoxy)piperidine-1-carboxylate (860 mg, 3.51 mmol, 1 equiv.) and 2-(tributyl-λ5-phosphanylidene)acetonitrile (1.02 g, 4.21 mmol, 1.2 equiv.) in toluene (20 mL) was degassed, purged with N₂, and stirred at 120° C. for 12 hours under N₂ atmosphere. The reaction mixture was filtered and concentrated under reduced pressure to give a residue. The residue was purified by flash silica gel chromatography (Eluent of 0-23% ethyl acetate/petroleum ether) to give tert-butyl 4-(2-(3-bromo-2-methylphenoxy)ethoxy)piperidine-1-carboxylate (800 mg, 1.7 mmol, 49.5% yield, 90% purity) as a yellow oil.

¹H NMR (400 MHz, CDCl₃) δ=7.17 (d, J=8.0 Hz, 1H), 7.00 (t, J=8.0 Hz, 1H), 6.79 (d, J=8.4 Hz, 1H), 4.12-4.09 (m, 2H), 3.85-3.83 (m, 2H), 3.79-3.71 (m, 2H), 3.60-3.51 (m, 1H), 3.16-3.09 (m, 2H), 2.33 (s, 3H), 1.86-1.83 (m, 2H), 1.60-1.54 (m, 2H), 1.47 (s, 9H).

### Step B. Procedure for Preparation of 4-(2-(3-bromo-2-methylphenoxy)ethoxy)piperidine

To a solution of tert-butyl 4-(2-(3-bromo-2-methylphenoxy)ethoxy)piperidine-1-carboxylate (800 mg, 1.93 mmol, 1 equiv.) was added HCl/dioxane (4 M, 965.41 μL, 2 equiv.). The mixture was stirred at 25° C. for 1 hour. The reaction mixture was filtered and concentrated under reduced pressure to give 4-(2-(3-bromo-2-methylphenoxy)ethoxy)piperidine (800 mg, crude) as a white solid.

### Step C. Procedure for Preparation of ethyl 2-(4-(2-(3-bromo-2-methylphenoxy)ethoxy)piperidin-1-yl)acetate

To a solution of 4-(2-(3-bromo-2-methylphenoxy)ethoxy)piperidine (400 mg, 1.14 mmol, 1 eq, HCl) in CH₃CN (4 mL) was added K₂CO₃ (472.95 mg, 3.42 mmol, 3 equiv.) and ethyl 2-bromoacetate (190.49 mg, 1.14 mmol, 126.15 μL, 1 equiv.). The mixture was stirred at 60° C. for 1 hour. The reaction mixture was filtered and concentrated under reduced pressure to give a residue. The residue was purified by flash silica gel chromatography (Eluent of 0˜70% ethyl acetate/petroleum ether) to give ethyl 2-(4-(2-(3-bromo-2-methylphenoxy)ethoxy)piperidin-1-yl)acetate (420 mg, 944.28 μmol, 82.7% yield, 90% purity) as a yellow oil.

MS (ESI) m/z: 402.0 [M+H]⁺.

¹H NMR (400 MHz, CDCl₃) δ=7.17 (d, J=8.0 Hz, 1H), 7.01 (t, J=8.2 Hz, 1H), 6.80 (d, J=8.0 Hz, 1H), 4.22 (q, J=6.8 Hz, 2H), 4.12 (t, J=4.4 Hz, 2H), 3.87-3.80 (m, 2H), 3.62 (s, 1H), 3.48 (t, J=6.0 Hz, 2H), 3.05 (s, 2H), 2.93-2.59 (m, 2H), 2.32 (s, 3H), 2.14-2.02 (m, 2H), 1.87 (s, 2H), 1.29 (t, J=6.8 Hz, 3H).

### Step D. Procedure for Preparation of tert-butyl 6-(8-(benzo[d]thiazol-2-ylcarbamoyl)-3,4-dihydroisoquinolin-2(1H)-yl)-3-(3-(2-((1-(2-ethoxy-2-oxoethyl)piperidin-4-yl)oxy)ethoxy)-2-methylphenyl)picolinate

A mixture of tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (679.40 mg, 998.24 μmol, 90% purity, 1.2 equiv.) and ethyl 2-(4-(2-(3-bromo-2-methylphenoxy)ethoxy)piperidin-1-yl)acetate (370 mg, 831.86 μmol, 90% purity, 1 equiv.) in dioxane (6 mL) was added KF (1.5 M, 1.66 mL, 3 equiv.) and Ad₂nBuP Pd G₃ (cataCXium® A Pd G₃) (60.58 mg, 83.19 μmol, 0.1 equiv.). After addition, the mixture was degassed and purged with N₂ and stirred at 100° C. for 2 hours under microwave. The reaction mixture was filtered and concentrated under reduced pressure to give a residue. The residue was purified by flash silica gel chromatography (Eluent of 0-65% dichloromethane/methanol) to give tert-butyl 6-(8-(benzo[d]thiazol-2-ylcarbamoyl)-3,4-dihydroisoquinolin-2(1H)-yl)-3-(3-(2-((1-(2-ethoxy-2-oxoethyl)piperidin-4-yl)oxy)ethoxy)-2-methylphenyl)picolinate (600 mg, 669.9 μmol, 80.5% yield, 90% purity) as a yellow solid.

MS (ESI) m/z: 806.5 [(M−56)/2+375.6]⁺.

¹H NMR (400 MHz, CDCl₃) δ=10.47-9.88 (m, 1H), 7.86 (d, J=8.0 Hz, 1H), 7.62-7.55 (m, 2H), 7.38-7.28 (m, 5H), 7.10 (t, J=8.0 Hz, 1H), 6.90 (d, J=8.8 Hz, 1H), 6.84 (d, J=8.0 Hz, 1H), 6.72 (d, J=7.2 Hz, 1H), 5.15-4.90 (m, 2H), 4.18 (q, J=7.2 Hz, 2H), 4.12-4.09 (m, 4H), 3.84 (t, J=5.2 Hz, 2H), 3.51-3.46 (m, 1H), 3.20 (s, 2H), 3.07 (t, J=5.6 Hz, 2H), 2.81 (s, 2H), 2.40-2.29 (m, 2H), 2.00 (s, 3H), 1.96-1.90 (m, 2H), 1.77-1.70 (m, 2H), 1.27 (t, J=7.2 Hz, 3H), 1.15 (s, 9H).

### Step E. Procedure for Preparation of 2-[4-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]ethoxy]-1-piperidyl]acetic acid

To a solution of tert-butyl 6-(8-(benzo[d]thiazol-2-ylcarbamoyl)-3,4-dihydroisoquinolin-2(1H)-yl)-3-(3-(2-((1-(2-ethoxy-2-oxoethyl)piperidin-4-yl)oxy)ethoxy)-2-methylphenyl)picolinate (600 mg, 699.77 μmol, 94% purity, 1 equiv.) in THE (6 mL) was added LiOH·H₂O (88.09 mg, 2.10 mmol, 3 equiv.) and H₂O (1.5 mL) at 25° C. The mixture was stirred at 25° C. for 12 hours. The reaction mixture was concentrated under reduced pressure to remove THF. The aqueous phase was adjusted to pH=4-5 with 1M HCl. The reaction mixture was filtered. After filtering, the filter cake was diluted in ethyl acetate. The combined organic layers were dried over Na₂SO₄, filtered, and concentrated under reduced pressure to give 2-[4-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]ethoxy]-1-piperidyl]acetic acid (800 mg, crude) as a yellow solid.

MS (ESI) m/z: 778.4 [M+H]⁺

### Step F. Procedure for Preparation of tert-butyl 6-[8-(1,3-benzothiazol-2-ylcarbamoyl)-3,4-dihydro-1H-isoquinolin-2-yl]-3-[3-[2-[[1-[2-[[3-(2,6-dioxo-3-piperidyl)-1-methyl-indazol-7-yl]amino]-2-oxo-ethyl]-4-piperidyl]oxy]ethoxy]-2-methyl-phenyl]pyridine-2-carboxylate

To a solution of 2-[4-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]ethoxy]-1-piperidyl]acetic acid (100 mg, 128.55 μmol, 1 equiv.) and 3-(7-amino-1-methyl-indazol-3-yl)piperidine-2,6-dione (33.20 mg, 128.55 μmol, 1 equiv.) in pyridine (2 mL) was added EDCI (36.96 mg, 192.82 μmol, 1.5 equiv.). The mixture was stirred at 25° C. for 2 hours. The reaction mixture was concentrated under reduced pressure to give tert-butyl 6-[8-(1,3-benzothiazol-2-ylcarbamoyl)-3,4-dihydro-1H-isoquinolin-2-yl]-3-[3-[2-[[1-[2-[[3-(2,6-dioxo-3-piperidyl)-1-methyl-indazol-7-yl]amino]-2-oxo-ethyl]-4-piperidyl]oxy]ethoxy]-2-methyl-phenyl]pyridine-2-carboxylate (100 mg, 98.2 μmol, 76.4% yield) as a yellow solid.

MS (ESI) m/z: 1018.7[M+H]⁺

### Step G. Procedure for Preparation of 6-[8-(1,3-benzothiazol-2-ylcarbamoyl)-3,4-dihydro-1H-isoquinolin-2-yl]-3-[3-[2-[[1-[2-[[3-(2,6-dioxo-3-piperidyl)-1-methyl-indazol-7-yl]amino]-2-oxo-ethyl]-4-piperidyl]oxy]ethoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[2-[[1-[2-[[3-(2,6-dioxo-3-piperidyl)-1-methyl-indazol-7-yl]amino]-2-oxo-ethyl]-4-piperidyl]oxy]ethoxy]-2-methyl-phenyl]pyridine-2-carboxylate (100 mg, 98.21 μmol, 1 equiv.) in DCM (0.5 mL) was added TFA (641.67 mg, 5.63 mmol, 416.67 μL, 57.30 equiv.). The mixture was stirred at 25° C. for 12 hours. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[2-[[1-[2-[[3-(2,6-dioxo-3-piperidyl)-1-methyl-indazol-7-yl]amino]-2-oxo-ethyl]-4-piperidyl]oxy]ethoxy]-2-methyl-phenyl]pyridine-2-carboxylic acid (41.4 mg, 43.0 μmol, 43.8% yield, 97.6% purity) as a white solid.

MS (ESI) m/z: 962.6 [M+H]⁺.

¹H NMR (400 MHz, DMSO-d₆) δ=13.03-12.38 (m, 2H), 10.89 (s, 1H), 9.87 (s, 1H), 8.14 (s, 1H), 8.03 (d, J=8.0 Hz, 1H), 7.79 (d, J=8.0 Hz, 1H), 7.62 (d, J=7.2 Hz, 1H), 7.56 (d, J=8.0 Hz, 1H), 7.50-7.42 (m, 3H), 7.38-7.32 (m, 2H), 7.29 (d, J=7.2 Hz, 1H), 7.12-7.04 (m, 2H), 6.97 (d, J=8.8 Hz, 1H), 6.91 (d, J=8.0 Hz, 1H), 6.65 (d, J=7.6 Hz, 1H), 4.98 (s, 2H), 4.37 (dd, J=5.2, 10.4 Hz, 1H), 4.12-4.05 (m, 5H), 3.92 (t, J=6.0 Hz, 2H), 3.81-3.75 (m, 2H), 3.48-3.43 (m, 2H), 3.17 (s, 2H), 3.02 (t, J=5.2 Hz, 2H), 2.86-2.79 (m, 2H), 2.68-2.60 (m, 2H), 2.38-2.32 (m, 2H), 2.19-2.13 (m, 1H), 1.91 (s, 5H), 1.67-1.56 (m, 2H).

### Example 156. Preparation of Compound 195

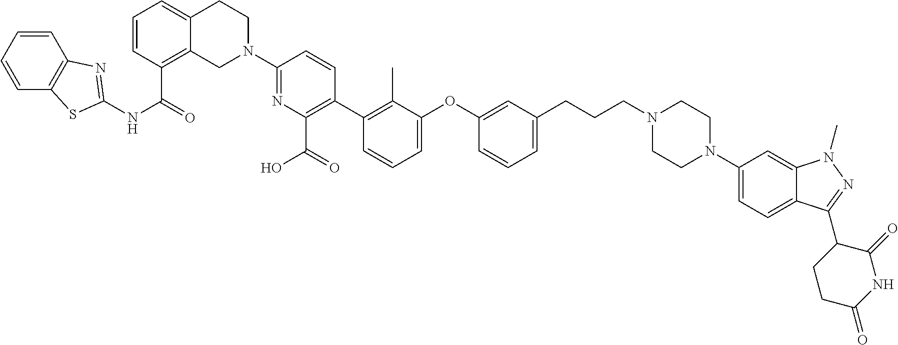 [SMILES: Cc1c(Oc2cccc(CCCN3CCN(c4ccc5c(C6CCC(=O)NC6=O)nn(C)c5c4)CC3)c2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[3-[4-[3-(2,6-dioxo-3-piperidyl)-1-methyl-indazol-6-yl]piperazin-1-yl]propyl]phenoxy]-2-methyl-phenyl]pyridine-2-carboxylic acid

### Step A. Procedure for Preparation of ethyl 3-(3-(3-bromo-2-methylphenoxy)phenyl)propanoate

A mixture of ethyl 3-(3-hydroxyphenyl)propanoate (1 g, 5.15 mmol, 1 equiv.), 1,3-dibromo-2-methylbenzene (2.57 g, 10.30 mmol, 2 equiv.), 2,2,6,6-tetramethylheptane-3,5-dione (237.19 mg, 1.29 mmol, 265.02 μL, 0.25 equiv.), and Cs₂CO₃ (2.52 g, 7.72 mmol, 1.5 equiv.) in NMP (10 mL) was added CuI (490.28 mg, 2.57 mmol, 0.5 equiv.). The reaction mixture was purged with N₂ and stirred at 120° C. for 15 hours under N₂ atmosphere. The reaction mixture was diluted with H₂O (10 mL) and extracted with ethyl acetate (10 mL×3). The combined organic layers were washed with sat. NaHCO₃ (10 mL×2), filtered, and concentrated under reduced pressure to give a residue. The residue was purified by flash silica gel chromatography (Eluent of 0˜12 ethyl acetate/petroleum ether) to give ethyl 3-(3-(3-bromo-2-methylphenoxy)phenyl)propanoate (650 mg, 1.7 mmol, 34.700 yield) as a yellow oil

¹H NMR (400 MHz, DMSO-d₆) δ=7.42 (d, J=8.0 Hz, 1H), 7.29-7.21 (m, 1H), 7.15 (t, J=8.0 Hz, 1H), 6.97 (d, J=7.6 Hz, 1H), 6.88 (d, J=8.0 Hz, 1H), 6.82-6.77 (m, 1H), 6.75-6.69 (m, 1H), 4.00 (q, J=7.2 Hz, 2H), 2.85-2.78 (m, 2H), 2.58 (t, J=7.6 Hz, 2H), 2.24 (s, 3H), 1.12 (t, J=7.2 Hz, 3H)

### Step B. Procedure for Preparation of 3-(3-(3-bromo-2-methylphenoxy)phenyl)propan-1-ol

A mixture of ethyl 3-(3-(3-bromo-2-methylphenoxy)phenyl)propanoate (650 mg, 1.79 mmol, 1 equiv.) in THE (7 mL) was slowly added LiAlH₄ (54.33 mg, 1.43 mmol, 0.8 equiv.) at 0° C., and then the mixture was stirred at 25° C. for 4 hours. The mixture was quenched by Na₂SO₄ (2 g). The solution was poured into ice-water (15 mL) slowly, and the pH was adjusted to 4-5 with 1 M HCl. The resulting mixture was extracted with EtOAc (15 mL×3), washed with brine (10 mL×2), dried by sodium sulfate, filtered, and concentrated under reduced pressure to give 3-(3-(3-bromo-2-methylphenoxy)phenyl)propan-1-ol (556 mg, 1.5 mmol, 84.2% yield, 87.1% purity) as a yellow oil.

¹H NMR (400 MHz, DMSO-d₆) δ=7.42 (d, J=8.0 Hz, 1H), 7.29-7.22 (m, 1H), 7.16 (t, J=8.0 Hz, 1H), 6.96 (d, J=7.6 Hz, 1H), 6.92-6.86 (m, 1H), 6.78 (s, 1H), 6.73-6.67 (m, 1H), 4.45 (t, J=5.2 Hz, 1H), 3.42-3.36 (m, 2H), 2.61-2.55 (m, 2H), 2.26 (s, 3H), 1.72-1.63 (m, 2H)

### Step C. Procedure for Preparation of 3-(3-(3-bromo-2-methylphenoxy)phenyl)propanal

A mixture of (COCl)₂ (439.41 mg, 3.46 mmol, 303.04 μL, 2 equiv.) in DCM (8 mL) at −78° C. was charged with DMSO (540.98 mg, 6.92 mmol, 540.98 μL, 4 equiv.) and stirred for 0.5 hours. The solution was charged with 3-(3-(3-bromo-2-methylphenoxy)phenyl)propan-1-ol (556 mg, 1.73 mmol, 1 equiv.) in DCM (4 mL) and stirred at −70° C. for 1 hour. Then the mixture was charged with TEA (1.05 g, 10.39 mmol, 1.45 mL, 6 equiv.), warmed to 25° C., and stirred 0.5 hours under N₂ atmosphere. The reaction mixture was diluted with H₂O (10 mL) and extracted with DCM (10 mL×3). The combined organic layers were washed with sat. NaCl (10 mL×2), filtered, and concentrated under reduced pressure to give 3-(3-(3-bromo-2-methylphenoxy)phenyl)propanal (293 mg, 728.8 μmol, 42.1% yield, 79.4% purity) as a yellow solid

¹H NMR (400 MHz, DMSO-d₆) δ=9.69 (s, 1H), 7.43 (d, J=8.0 Hz, 1H), 7.30-7.22 (m, 1H), 7.16 (t, J=8.0 Hz, 1H), 7.01-6.94 (m, 1H), 6.91-6.83 (m, 2H), 6.74-6.67 (m, 1H), 2.87-2.80 (m, 2H), 2.79-2.72 (m, 2H), 2.26 (s, 3H)

### Step D. Procedure for Preparation of tert-butyl 6-(8-(benzo[d]thiazol-2-ylcarbamoyl)-3,4-dihydroisoquinolin-2(1H)-yl)-3-(2-methyl-3-(3-(3-oxopropyl)phenoxy)phenyl)picolinate

3-(3-(3-Bromo-2-methylphenoxy)phenyl)propanal (210 mg, 525.67 μmol, 79.9% purity, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (354.20 mg, 578.24 μmol, 1.1 eq), K₂CO₃ (217.95 mg, 1.58 mmol, 1.13 mL, 3 equiv.), and Ad₂nBuP Pd G₃ (76.57 mg, 105.13 μmol, 0.2 equiv.) were taken up into a microwave tube in dioxane (2.4 mL) and H₂O (0.8 mL). The sealed tube was heated at 100° C. for 1 hour under microwave. The reaction mixture was partitioned between H₂O (10 mL) and ethyl acetate (8 mL×3). The organic phase was separated, washed with aqueous NaCl (5 mL×3), dried over (Na₂SO₄), filtered, and concentrated under reduced pressure to give a residue. The residue was purified by flash silica gel chromatography (Eluent of 0˜26% ethyl acetate/petroleum ether) to give tert-butyl 6-(8-(benzo[d]thiazol-2-ylcarbamoyl)-3,4-dihydroisoquinolin-2(1H)-yl)-3-(2-methyl-3-(3-(3-oxopropyl)phenoxy)phenyl)picolinate (120 mg, 132.7 μmol, 25.2% yield, 80.2% purity) as a yellow solid MS (ESI) m/z: 725.3 [M+H]⁺.

### Step E. Procedure for Preparation of tert-butyl 6-[8-(1,3-benzothiazol-2-ylcarbamoyl)-3,4-dihydro-1H-isoquinolin-2-yl]-3-[3-[3-[3-[4-[3-(2,6-dioxo-3-piperidyl)-1-methyl-indazol-6-yl]piperazin-1-yl]propyl]phenoxy]-2-methyl-phenyl]pyridine-2-carboxylate

To a solution of tert-butyl 6-(8-(benzo[d]thiazol-2-ylcarbamoyl)-3,4-dihydroisoquinolin-2(1H)-yl)-3-(2-methyl-3-(3-(3-oxopropyl)phenoxy)phenyl)picolinate (70 mg, 96.57 μmol, 1 equiv.) and 3-(1-methyl-6-piperazin-1-yl-indazol-3-yl)piperidine-2,6-dione (31.62 mg, 96.57 μmol, 1 equiv.) in DCM (3 mL) was added AcOH (5.80 mg, 96.57 μmol, 5.52 μL, 1 equiv.), and the mixture was stirred at 25° C. for 1 hour. Then NaBH(OAc)₃ (61.40 mg, 289.71 μmol, 3 equiv.) was added to the mixture. The resulting mixture was stirred at 25° C. for 2 hours. The reaction mixture was concentrated under reduced pressure to give tert-butyl 6-[8-(1,3-benzothiazol-2-ylcarbamoyl)-3,4-dihydro-1H-isoquinolin-2-yl]-3-[3-[3-[3-[4-[3-(2,6-dioxo-3-piperidyl)-1-methyl-indazol-6-yl]piperazin-1-yl]propyl]phenoxy]-2-methyl-phenyl]pyridine-2-carboxylate (80 mg, crude) as a green oil.

MS (ESI) m/z: 519.1 [M12+H]⁺.

### Step F. Procedure for Preparation of 6-[8-(1,3-benzothiazol-2-ylcarbamoyl)-3,4-dihydro-1H-isoquinolin-2-yl]-3-[3-[3-[3-[4-[3-(2,6-dioxo-3-piperidyl)-1-methyl-indazol-6-yl]piperazin-1-yl]propyl]phenoxy]-2-methyl-phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[3-[3-[4-[3-(2,6-dioxo-3-piperidyl)-1-methyl-indazol-6-yl]piperazin-1-yl]propyl]phenoxy]-2-methyl-phenyl]pyridine-2-carboxylate (80 mg, 77.20 μmol, 1 equiv.) in DCM (1 mL) and TFA (1 mL) was stirred at 25° C. for 16 hours. The reaction mixture was concentrated under reduced pressure to remove solvent to give a residue. The residue was purified by prep-HPLC to give 6-[8-(1,3-benzothiazol-2-ylcarbamoyl)-3,4-dihydro-1H-isoquinolin-2-yl]-3-[3-[3-[3-[4-[3-(2,6-dioxo-3-piperidyl)-1-methyl-indazol-6-yl]piperazin-1-yl]propyl]phenoxy]-2-methyl-phenyl]pyridine-2-carboxylic acid (20.4 mg, 20.5 μmol, 26.5% yield, 98.1% purity) as a white solid.

MS (ESI) m/z: 980.9 [M+H]⁺.

¹H NMR (400 MHz, DMSO-d₆) δ=13.16-12.60 (m, 1H), 10.85 (s, 1H), 8.13 (s, 1H), 8.01 (d, J=8.0 Hz, 1H), 7.78 (d, J=8.0 Hz, 1H), 7.61 (d, J=7.2 Hz, 1H), 7.54-7.43 (m, 3H), 7.41-7.30 (m, 3H), 7.27-7.17 (m, 2H), 7.02-6.81 (m, 7H), 6.74 (d, J=8.4 Hz, 1H), 6.68 (s, 1H), 4.95 (s, 2H), 4.26 (d, J=5.2, 8.8 Hz, 1H), 3.88 (s, 6H), 2.91 (s, 4H), 2.72 (d, J=1.6 Hz, 2H), 2.64-2.55 (m, 6H), 2.38-2.23 (m, 2H), 2.19-2.12 (m, 1H), 1.96-1.75 (m, 6H).

### Example 157. Preparation of Compound 201

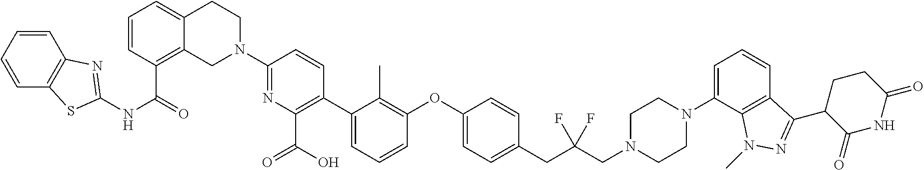 [SMILES: Cc1c(Oc2ccc(CC(F)(F)CN3CCN(c4cccc5c(C6CCC(=O)NC6=O)nn(C)c45)CC3)cc2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-7-yl]piperazin-1-yl]-2,2-difluoro-propyl]phenoxy]-2-methyl-phenyl]pyridine-2-carboxylic acid

### Step A. Procedure for Preparation of 3-(4-(3-bromo-2-methylphenoxy)phenyl)-2,2-difluoropropan-1-ol

To a solution of ethyl 3-(4-(3-bromo-2-methylphenoxy)phenyl)-2,2-difluoropropanoate (1 g, 2.50 mmol, 1 equiv.) in THF (10 mL) was added LiAlH₄ (95.07 mg, 2.50 mmol, 1 equiv.). The mixture was stirred at 25° C. for 2 hours. The reaction mixture was quenched by addition H₂O (1.8 mL), 15% NaOH (1.8 mL), and H₂O 5.4 mL. The resulting mixture was extracted with ethyl acetate (10 mL×3). The combined organic layers were dried over Na₂SO₄, filtered, and concentrated under reduced pressure to give a residue. The residue was purified by flash silica gel chromatography (Eluent of 0˜10% ethyl acetate/petroleum ether) to give 3-(4-(3-bromo-2-methylphenoxy)phenyl)-2,2-difluoropropan-1-ol (530 mg, 1.1 mmol, 47.3% yield, 80% purity) as a colorless oil.

¹H NMR (400 MHz, DMSO-d₆) δ=7.44 (d, J=8.0 Hz, 1H), 7.27 (d, J=8.4 Hz, 2H), 7.17 (t, J=8.1 Hz, 1H), 6.93-6.87 (m, 3H), 5.61-5.57 (m, 1H), 3.56-3.50 (m, 2H), 3.24-3.17 (m, 2H), 2.26 (s, 3H)

F NMR (400 MHz, DMSO-d₆) δ=−105.73.

### Step B. Procedure for Preparation of 3-(4-(3-bromo-2-methylphenoxy)phenyl)-2,2-difluoropropyl trifluoromethanesulfonate

To a solution of 3-(4-(3-bromo-2-methylphenoxy)phenyl)-2,2-difluoropropan-1-ol (100 mg, 279.96 μmol, 1 equiv.), Na₂SO₄ (71.58 mg, 503.93 μmol, 51.13 μL, 1.8 equiv.), and pyridine (33.22 mg, 419.95 μmol, 33.90 μL, 1.5 equiv.) in DCM (5 mL) was added trifluoromethylsulfonyl trifluoromethanesulfonate (118.48 mg, 419.95 μmol, 69.29 μL, 1.5 equiv.). The mixture was stirred at 0° C. for 1 hour. The reaction mixture was diluted with H₂O (2 mL) and extracted with DCM (10 mL). The combined organic layers were washed with 1N citric acid (2 mL) and 1N NaHCO₃ (2 mL), dried over Na₂SO₄, filtered, and concentrated under reduced pressure to give a residue. The residue was used for next step without further purification. The compound 3-(4-(3-bromo-2-methylphenoxy)phenyl)-2,2-difluoropropyl trifluoromethanesulfonate (130 mg, crude) was obtained as a colorless oil.

### Step C. Procedure for Preparation of 3-[7-[4-[3-[4-(3-bromo-2-methyl-phenoxy)phenyl]-2,2-difluoro-propyl]piperazin-1-yl]-1-methyl-indazol-3-yl]piperidine-2,6-dione

A mixture of [3-[4-(3-bromo-2-methyl-phenoxy)phenyl]-2,2-difluoro-propyl]trifluoromethanesulfonate (120 mg, 245.27 μmol, 1 equiv.), 3-(1-methyl-7-piperazin-1-yl-indazol-3-yl)piperidine-2,6-dione (64.24 mg, 196.22 μmol, 0.8 equiv.), K₂CO₃ (84.75 mg, 613.18 μmol, 2.5 equiv.), and KI (20.36 mg, 122.64 μmol, 0.5 equiv.) in CH₃CN (1.5 mL) was stirred at 50° C. for 10 hours. The reaction mixture was filtered and concentrated under reduced pressure to give a residue. The residue was purified by flash silica gel chromatography (Eluent of 0˜20% ethyl acetate/petroleum ether) to give 3-[7-[4-[3-[4-(3-bromo-2-methyl-phenoxy)phenyl]-2,2-difluoro-propyl]piperazin-1-yl]-1-methyl-indazol-3-yl]piperidine-2,6-dione (40 mg, 60.0 μmol, 24.4% yield) as a colorless oil.

MS (ESI) m/z: 668.3 [M+H]⁺.

### Step D. Procedure for Preparation of tert-butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-6-yl]piperazin-1-yl]-2,2-difluoro-propyl]phenoxy]-2-methyl-phenyl]pyridine-2-carboxylate

A mixture of 3-[7-[4-[3-[4-(3-bromo-2-methyl-phenoxy)phenyl]-2,2-difluoro-propyl]piperazin-1-yl]-1-methyl-indazol-3-yl]piperidine-2,6-dione (40.00 mg, 60.01 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (51.46 mg, 84.01 μmol, 1.4 equiv.), [2-(2-aminophenyl)phenyl]palladium(1⁺); bis(1-adamantyl)-butyl-phosphane; methanesulfonate (8.74 mg, 12.00 μmol, 0.2 equiv.), and KF (1.5 M, 60.01 μL, 1.5 equiv.) in dioxane (0.4 mL) was degassed and purged with N₂ three times, and then the mixture was stirred at 100° C. for 1 hour under microwave. The reaction mixture was concentrated under reduced pressure to give a residue. The residue was purified by flash silica gel chromatography (Eluent of 0˜100% ethyl acetate/petroleum ether) to give tert-butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-6-yl]piperazin-1-yl]-2,2-difluoro-propyl]phenoxy]-2-methyl-phenyl]pyridine-2-carboxylate (60 mg, 55.9 μmol, 93.2% yield) as a yellow solid.

MS (ESI) m/z: 1072.6 [M+H]⁺.

### Step E. Procedure for Preparation of 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-7-yl]piperazin-1-yl]-2,2-difluoro-propyl]phenoxy]-2-methyl-phenyl]pyridine-2-carboxylic acid

tert-Butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-6-yl]piperazin-1-yl]-2,2-difluoro-propyl]phenoxy]-2-methyl-phenyl]pyridine-2-carboxylate (60 mg, 55.96 μmol, 1 equiv.) in TFA (0.5 mL) and DCM (0.5 mL) was stirred at 40° C. for 1 hour. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-7-yl]piperazin-1-yl]-2,2-difluoro-propyl]phenoxy]-2-methyl-phenyl]pyridine-2-carboxylic acid (29.9 mg, 28.2 μmol, 50.4% yield, 95.9% purity) as an off-white solid.

MS (ESI) m/z: 1016.9 [M+H]⁺.

¹H NMR (400 MHz, DMSO-d₆) δ=12.97-12.50 (m, 2H), 10.89 (s, 1H), 8.03 (d, J=8.0 Hz, 1H), 7.79 (d, J=8.0 Hz, 1H), 7.62 (d, J=7.2 Hz, 1H), 7.54 (d, J=8.8 Hz, 1H), 7.49-7.44 (m, 2H), 7.41-7.33 (m, 3H), 7.28 (d, J=8.4 Hz, 2H), 7.20 (t, J=7.6 Hz, 1H), 7.08-6.99 (m, 3H), 6.96-6.79 (m, 4H), 4.99 (s, 2H), 4.34 (dd, J=5.2, 9.6 Hz, 1H), 4.23 (s, 3H), 3.93 (t, J=5.6 Hz, 2H), 3.29-3.24 (m, 2H), 3.15-2.85 (m, 8H), 2.80-2.60 (m, 6H), 2.35-2.12 (m, 2H), 1.89 (s, 3H).

F NMR (400 MHz, DMSO-d₆) δ=−96.31

### Example 158. Preparation of Compound 204a

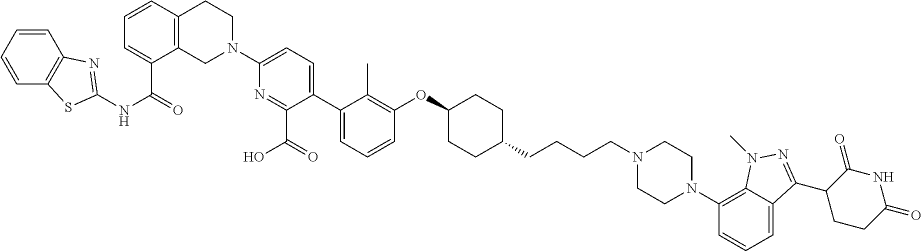 [SMILES: Cc1c(O[C@H]2CC[C@H](CCCCN3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-7-yl)piperazin-1-yl)butyl)cyclohexyl)oxy)-2-methylphenyl)picolinic acid

### Step A. Procedure for Preparation of 1-(((1r,4r)-4-((E)-4-(benzyloxy)but-1-en-1-yl)cyclohexyl)oxy)-3-bromo-2-methylbenzene

A mixture of 3-benzyloxypropyl(triphenyl)phosphonium; bromide (5.56 g, 11.31 mmol, 1.2 equiv.) in THE (30 mL) was added LiHMDS (1 M, 12.25 mL, 1.3 equiv.) at −70° C. under N₂ atmosphere for 1 hour. Then 4-(3-bromo-2-methyl-phenoxy)cyclohexanecarbaldehyde (2.8 g, 9.42 mmol, 1 equiv.) was added to the mixture at −70° C. under N₂ atmosphere. The mixture was stirred at 25° C. for 11 hours under N₂ atmosphere. The reaction mixture was quenched by addition NH₄Cl (100 mL) at 0° C., diluted with H₂O (80 mL), and extracted with ethyl acetate (80 mL×3). The combined organic layers were washed with aqueous NaCl (100 mL×3), dried over Na₂SO₄, filtered, and concentrated under reduced pressure to give a residue. The residue was purified by column chromatography (SiO₂, petroleum ether/ethyl acetate=30/1 to 10/1) to give 1-(((1r,4r)-4-((E)-4-(benzyloxy)but-1-en-1-yl)cyclohexyl)oxy)-3-bromo-2-methylbenzene (3 g, 6.9 mmol, 74.1% yield) as a yellow oil.

¹H NMR (400 MHz, CDCl₃) δ=7.21-7.12 (m, 5H), 6.98 (d, J=8.0 Hz, 1H), 6.81 (t, J=8.0 Hz, 1H), 6.65 (d, J=8.0 Hz, 1H), 5.25-5.06 (m, 2H), 4.42-4.33 (m, 2H), 4.00-3.87 (m, 1H), 3.33 (t, J=6.8 Hz, 2H), 2.29-2.21 (m, 2H), 2.14 (s, 3H), 1.97 (d, J=10.4 Hz, 2H), 1.66-1.50 (m, 2H), 1.41-1.26 (m, 3H), 1.10-0.97 (m, 2H)

### Step B. Procedure for Preparation of 4-((1r,4s)-4-(3-bromo-2-methylphenoxy)cyclohexyl)butan-1-ol

To a solution of 1-(((1r,4r)-4-((E)-4-(benzyloxy)but-1-en-1-yl)cyclohexyl)oxy)-3-bromo-2-methylbenzene (2.6 g, 6.06 mmol, 1 equiv.) in EtOAc (20 mL) was added PtO₂ (1.37 g, 6.06 mmol, 1.0 equiv.) under N₂ atmosphere. The suspension was degassed and purged with H₂. The mixture was stirred under H₂ (15 Psi) at 40° C. for 12 hours. The reaction mixture was filtered and concentrated under reduced pressure to give a residue. The reaction mixture was filtered and concentrated under reduced pressure to give a residue. The residue was purified by prep-HPLC to give 4-((1r,4s)-4-(3-bromo-2-methylphenoxy)cyclohexyl)butan-1-ol (1 g, 2.9 mmol, 48.3% yield) as a yellow oil.

¹H NMR (400 MHz, CDCl₃) δ=7.06 (d, J=7.6 Hz, 1H), 6.89 (t, J=8.0 Hz, 1H), 6.76-6.69 (m, 1H), 4.06-3.97 (m, 1H), 3.58 (t, J=6.8 Hz, 2H), 2.22 (s, 3H), 2.08-2.01 (m, 2H), 1.80-1.74 (m, 2H), 1.51-1.47 (m, 2H), 1.39-1.28 (m, 4H), 1.23-1.14 (m, 3H), 1.00-0.88 (m, 2H)

### Step C. Procedure for Preparation of 4-((1r,4s)-4-(3-bromo-2-methylphenoxy)cyclohexyl)butanal

To a solution of oxalyl dichloride (371.92 mg, 2.93 mmol, 256.50 uL, 2 equiv.) in DCM (5 mL) was added dropwise to a solution of DMSO (457.86 mg, 5.86 mmol, 457.86 uL, 4 equiv.) in DCM (5 mL) at −70° C. under N₂ atmosphere. The mixture was stirred at −70° C. for 30 minutes. After which time, 4-((1r,4s)-4-(3-bromo-2-methylphenoxy)cyclohexyl)butan-1-ol (500 mg, 1.47 mmol, 1 equiv.) in DCM (5 mL) was added dropwise at −70° C. The solution was stirred for 30 minutes at −70° C. Then TEA (889.50 mg, 8.79 mmol, 1.22 mL, 6 equiv.) was added into the solution. The solution was stirred at −70° C. for 2 hour under N₂ atmosphere. The reaction was diluted with water (50 mL) and extracted with DCM (80 mL×2). The combined organic layers were washed with brine (60 mL×1), dried over Na₂SO₄, filtered, and concentrated under reduced pressure to give a residue. The residue was purified by column chromatography (SiO₂, petroleum ether/ethyl acetate=100/1 to 15/1) to give 4-((1r,4s)-4-(3-bromo-2-methylphenoxy)cyclohexyl)butanal (400 mg, 1.1 mmol, 80.4% yield) as a yellow oil.

### Step D. Procedure for Preparation of tert-butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate

A mixture of tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (400 mg, 653.01 μmol, 1 equiv.), 4-((1r,4s)-4-(3-bromo-2-methylphenoxy)cyclohexyl)butanal (243.70 mg, 718.31 μmol, 1.1 equiv.), [2-(2-aminophenyl)phenyl]palladium (1⁺); bis(1-adamantyl)-butyl-phosphane; methanesulfonate (47.56 mg, 65.30 μmol, 0.1 equiv.), and K₂CO₃ (270.75 mg, 1.96 mmol, 3 equiv.) in dioxane (4 mL) and H₂O (1 mL) was degassed and purged with N₂ and stirred at 100° C. for 1 hour under N₂ atmosphere. The reaction mixture was concentrated under reduced pressure to give a residue. The residue was purified by column chromatography (SiO₂, petroleum ether/ethyl acetate=20/1 to 2/1) to give tert-butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate (120 mg, 161.0 μmol, 24.6% yield) as a yellow solid

MS (ESI) m/z: 745.5 [M+H]⁺.

### Step E. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-7-yl)piperazin-1-yl)butyl)cyclohexyl)oxy)-2-methylphenyl)picolinate

To a solution of 3-(1-methyl-7-piperazin-1-yl-indazol-3-yl)piperidine-2,6-dione (52.74 mg, 161.09 μmol, 1.2 equiv.) in DCM (1 mL) was added NaBH(OAc)₃ (85.35 mg, 402.72 μmol, 3 equiv.), AcOH (16.12 mg, 268.48 μmol, 15.35 μL, 2 equiv.), and tert-butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate (100 mg, 134.24 μmol, 1 equiv.) at 0° C. The mixture was stirred at 25° C. for 1 hour. The reaction was diluted with water (20 mL) and extracted with DCM (20 mL×2). The combined organic layers were washed with brine (30 mL×1), dried over Na₂SO₄, filtered, and concentrated under reduced pressure to give tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-7-yl)piperazin-1-yl)butyl)cyclohexyl)oxy)-2-methylphenyl)picolinate (120 mg, 113.6 μmol, 84.6% yield) as a yellow solid

MS (ESI) m/z: 1056.9 [M+H]⁺.

### Step F. Procedure for Preparation of 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-7-yl)piperazin-1-yl)butyl)cyclohexyl)oxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-7-yl)piperazin-1-yl)butyl)cyclohexyl)oxy)-2-methylphenyl)picolinate (120 mg, 113.60 μmol, 1 equiv.) in DCM (1 mL) was added TFA (1.54 g, 13.51 mmol, 1 mL, 118.89 equiv.). The mixture was stirred at 40° C. for 1 hour. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-7-yl)piperazin-1-yl)butyl)cyclohexyl)oxy)-2-methylphenyl)picolinic acid (52.3 mg, 48.2 μmol, 42.4% yield, 96.4% purity, FA) as a yellow solid.

MS (ESI) m/z: 1000.9 [M+H]⁺.

¹H NMR (400 MHz, DMSO-d₆) δ=13.01-12.71 (m, 1H), 10.88 (s, 1H), 8.13 (s, 1H), 8.03 (d, J=8.0 Hz, 1H), 7.78 (d, J=8.0 Hz, 1H), 7.62 (d, J=7.2 Hz, 1H), 7.49-7.41 (m, 4H), 7.40-7.31 (m, 2H), 7.10-7.01 (m, 3H), 6.98-6.89 (m, 2H), 6.61 (d, J=7.6 Hz, 1H), 4.97 (s, 2H), 4.37-4.30 (m, 1H), 4.24 (s, 3H), 4.19 (s, 1H), 3.91 (t, J=5.6 Hz, 2H), 3.26-3.18 (m, 4H), 3.02 (t, J=5.6 Hz, 2H), 2.68-2.61 (m, 2H), 2.57-2.51 (m, 4H), 2.36-2.29 (m, 1H), 2.20-2.09 (m, 2H), 2.07 (s, 2H), 1.87 (s, 3H), 1.83-1.74 (m, 2H), 1.62-1.50 (m, 2H), 1.45-1.19 (m, 8H), 1.12-1.00 (m, 2H)

### Example 159. Preparation of Compound 206

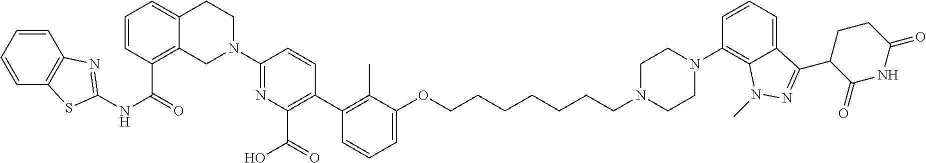 [SMILES: Cc1c(OCCCCCCCN2CCN(c3cccc4c(C5CCC(=O)NC5=O)nn(C)c34)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[7-[4-[3-(2,6-dioxo-3-piperidyl)-1-methyl-indazol-7-yl]piperazin-1-yl]heptoxy]-2-methyl-phenyl]pyridine-2-carboxylic acid

### Step A. Procedure for Preparation of tert-butyl 4-[3-(2,6-dibenzyloxy-3-pyridyl)-1-methyl-indazol-7-yl]piperazine-1-carboxylate

To a solution of 7-bromo-3-(2,6-dibenzyloxy-3-pyridyl)-1-methyl-indazole (5 g, 9.99 mmol, 1 equiv.) in dioxane (100 mL) was added Pd2(dba)3 (915.01 mg, 999.23 μmol, 0.1 equiv.), RuPhos (932.56 mg, 2.00 mmol, 0.2 eq) and Cs₂CO₃ (9.77 g, 29.98 mmol, 3 equiv.) then then tert-butyl piperazine-1-carboxylate (5.58 g, 29.98 mmol, 3 equiv.) was added in the mixture. The suspension was degassed and purged with N₂ and stirred under N₂ at 100° C. for 3 hours. The reaction mixture was concentrated under reduced pressure to remove solvent. Then diluted with H₂O 100 mL and extracted with ethyl acetate 300 mL (100 mL×3). The combined organic layers were washed with H₂O 300 mL (100 mL×3), dried over Na₂SO₄, filtered and concentrated under reduced pressure to give a residue. The residue was purified by column chromatography (SiO₂, petroleum ether/ethyl acetate=100/1 to 4/1). The compound tert-butyl 4-[3-(2,6-dibenzyloxy-3-pyridyl)-1-methyl-indazol-7-yl]piperazine-1-carboxylate (5.6 g, 8.6 mmol, 86.9% yield, 94% purity) was obtained as a yellow oil.

MS (ESI) m/z: 606.5 [M+H]⁺.

### Step B. Procedure for Preparation of tert-butyl 4-[3-(2,6-dioxo-3-piperidyl)-1-methyl-indazol-7-yl] piperazine-1-carboxylate

To a solution of tert-butyl 4-[3-(2,6-dibenzyloxy-3-pyridyl)-1-methyl-indazol-7-yl]piperazine-1-carboxylate (5.6 g, 9.25 mmol, 1 equiv.) in EtOH (50 mL) and THE (50 mL) was added AcOH (555.17 mg, 9.25 mmol, 528.73 μL, 1 equiv.), Pd(OH)₂ (5 g, 7.12 mmol, 20% purity, 0.77 equiv.) and Pd/C (5 g, 4.63 mmol, 10% purity, 0.5 equiv.) under N₂ atmosphere. The suspension was degassed and purged with H₂ three times. The mixture was stirred under H₂ (50 Psi) at 50° C. for 12 hours. The reaction mixture was filtered and concentrated under reduced pressure to remove solvent. The compound tert-butyl 4-[3-(2,6-dioxo-3-piperidyl)-1-methyl-indazol-7-yl]piperazine-1-carboxylate (1.3 g, crude) was obtained as a white solid.

MS (ESI) m/z: 428.3 [M+H]⁺.

### Step C. Procedure for Preparation of 3-(1-methyl-7-piperazin-1-yl-indazol-3-yl)piperidine-2, 6-dione

To a solution of tert-butyl 4-[3-(2,6-dioxo-3-piperidyl)-1-methyl-indazol-7-yl]piperazine-1-carboxylate (1 g, 2.34 mmol, 1 equiv.) was added HCl/dioxane (4 M, 584.80 μL, 1 equiv.). The mixture was stirred at 25° C. for 1 hour. The reaction mixture was concentrated under reduced pressure to remove solvent. The compound 3-(1-methyl-7-piperazin-1-yl-indazol-3-yl)piperidine-2, 6-dione (500 mg, crude, HCl) was obtained as a gray solid.

### Step D. Procedure for Preparation of 7-(3-bromo-2-methyl-phenoxy)heptanal

To a solution of DMSO (830.02 mg, 10.62 mmol, 830.02 uL, 4 equiv.) in DCM (10 mL) was added (COCl)₂ (674.22 mg, 5.31 mmol, 464.98 μL, 2 equiv.) at −70° C. for 1 hour, after addition, 7-(3-bromo-2-methyl-phenoxy)heptan-1-ol (800 mg, 2.66 mmol, 1 equiv.) was added at −70° C. The reaction mixture was stirred at −70° C. for 1 hour, Et₃N (1.61 g, 15.94 mmol, 2.22 mL, 6 equiv.) was added at −70° C., the reaction mixture was stirred at −70° C. for 1 hour. The mixture solution diluted with H₂O 10 mL and extracted with DCM 30 mL (10 mL×3). The combined organic layers were washed with H₂O 30 mL (10 mL×3), dried over Na₂SO₄, filtered and concentrated under reduced pressure to give a residue. The residue was purified by column chromatography (SiO₂, petroleum ether/ethyl acetate=100/1 to 10/1). The compound 7-(3-bromo-2-methyl-phenoxy)heptanal (640 mg, 1.9 mmol, 72.4% yield, 90% purity) was obtained as a yellow oil.

### Step E. Procedure for Preparation of tert-butyl 6-[8-(1,3-benzothiazol-2-ylcarbamoyl)-3,4-dihydro-1H-isoquinolin-2-yl]-3-[2-methyl-3-(7-oxoheptoxy)phenyl]pyridine-2-carboxylate

To a solution of 7-(3-bromo-2-methyl-phenoxy)heptanal (203.52 mg, 680.22 μmol, 1 equiv.) in dioxane (5 mL) was added K₂CO₃ (141.02 mg, 1.02 mmol, 1.5 equiv.), di-tert-butyl(cyclopentyl) phosphane; dichloropalladium; iron (88.67 mg, 136.04 μmol, 0.2 equiv.) and tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl) pyridine-2-carboxylate (500 mg, 816.27 μmol, 1.2 equiv.). The mixture was stirred at 100° C. for 1 hour. The reaction mixture was concentrated under reduced pressure to remove solvent. The residue was purified by column chromatography (SiO₂, petroleum ether: ethyl acetate=2:1). The compound tert-butyl 6-[8-(1,3-benzothiazol-2-ylcarbamoyl)-3,4-dihydro-1H-isoquinolin-2-yl]-3-[2-methyl-3-(7-oxoheptoxy)phenyl]pyridine-2-carboxylate (250 mg, 329.1 μmol, 48.3% yield, 92.8% purity) was obtained as a yellow solid.

MS (ESI) m/z: 705.4 [M+H]⁺

### Step F. Procedure for Preparation of tert-butyl 6-[8-(1,3-benzothiazol-2-ylcarbamoyl)-3,4-dihydro-1H-isoquinolin-2-yl]-3-[3-[7-[4-[3-(2,6-dioxo-3-piperidyl)-1-methyl-indazol-7-yl]piperazin-1-yl]heptoxy]-2-methyl-phenyl]pyridine-2-carboxylate

To a solution of tert-butyl 6-[8-(1,3-benzothiazol-2-ylcarbamoyl)-3,4-dihydro-1H-isoquinolin-2-yl]-3-[2-methyl-3-(7-oxoheptoxy)phenyl]pyridine-2-carboxylate (100 mg, 141.87 μmol, 1 equiv.) in DCM (2 mL) was added 3-(1-methyl-7-piperazin-1-yl-indazol-3-yl)piperidine-2,6-dione (55.73 mg, 170.24 μmol, 1.2 equiv.) and NMM (43.05 mg, 425.61 μmol, 46.79 μL, 3 equiv.). The suspension was degassed and purged with N₂ and stirred under N₂ at 20° C. for 3 hours. Then the NaBH₃CN (26.75 mg, 425.61 μmol, 3 equiv.) was added in the mixture. The mixture was stirred under N₂ at 20° C. for 1 hour. The reaction mixture was concentrated under reduced pressure to remove solvent. The compound tert-butyl 6-[8-(1,3-benzothiazol-2-ylcarbamoyl)-3,4-dihydro-1H-isoquinolin-2-yl]-3-[3-[7-[4-[3-(2,6-dioxo-3-piperidyl)-1-methyl-indazol-7-yl]piperazin-1-yl]heptoxy]-2-methyl-phenyl]pyridine-2-carboxylate (100 mg, crude) was obtained as a yellow oil.

MS (ESI) m/z: 1016.8 [M+H]⁺

### Step G. Procedure for Preparation of 6-[8-(1,3-benzothiazol-2-ylcarbamoyl)-3,4-dihydro-1H-isoquinolin-2-yl]-3-[3-[7-[4-[3-(2,6-dioxo-3-piperidyl)-1-methyl-indazol-7-yl]piperazin-1-yl]heptoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[7-[4-[3-(2,6-dioxo-3-piperidyl)-1-methyl-indazol-7-yl]piperazin-1-yl]heptoxy]-2-methyl-phenyl] pyridine-2-carboxylate (80 mg, 78.72 μmol, 1 equiv.) in DCM (1.5 mL) was added TFA (8.98 mg, 78.72 μmol, 5.83 μL, 1 equiv.). The mixture was stirred at 25° C. for 12 hours. The reaction mixture was concentrated under reduced pressure to remove solvent. The residue was diluted with DMF (1.5 mL). The residue was purified by prep-HPLC. The compound 6-[8-(1,3-benzothiazol-2-ylcarbamoyl)-3,4-dihydro-1H-isoquinolin-2-yl]-3-[3-[7-[4-[3-(2,6-dioxo-3-piperidyl)-1-methyl-indazol-7-yl]piperazin-1-yl]heptoxy]-2-methyl-phenyl]pyridine-2-carboxylic acid (22.8 mg, 21.0 μmol, 26.7% yield, 92.7% purity, FA) was obtained as a pink solid.

MS (ESI) m/z: 960.5 [M+H]⁺.

¹H NMR (400 MHz, DMSO-d₆) δ=10.89 (s, 1H), 8.14 (s, 1H), 8.02 (d, J=7.6 Hz, 1H), 7.79 (d, J=8.0 Hz, 1H), 7.62 (d, J=7.2 Hz, 1H), 7.50-7.41 (m, 4H), 7.41-7.34 (m, 3H), 7.13-7.06 (m, 1H), 7.05-6.98 (m, 2H), 6.95 (d, J=8.8 Hz, 1H), 6.89 (d, J=8.4 Hz, 1H), 6.63 (d, J=8.0 Hz, 1H), 4.98 (s, 2H), 4.36-4.30 (m, 1H), 4.23 (s, 3H), 3.98 (d, J=5.6 Hz, 2H), 3.93-3.89 (m, 2H), 3.08-2.98 (m, 7H), 2.67 (d, J=2.0 Hz, 2H), 2.60 (s, 1H), 2.33 (d, J=2.0 Hz, 3H), 2.16 (d, J=7.2 Hz, 2H), 1.91 (s, 3H), 1.74 (d, J=6.8 Hz, 3H), 1.52-1.45 (m, 4H), 1.40-1.33 (m, 4H)

### Example 160. Preparation of Compound 207b

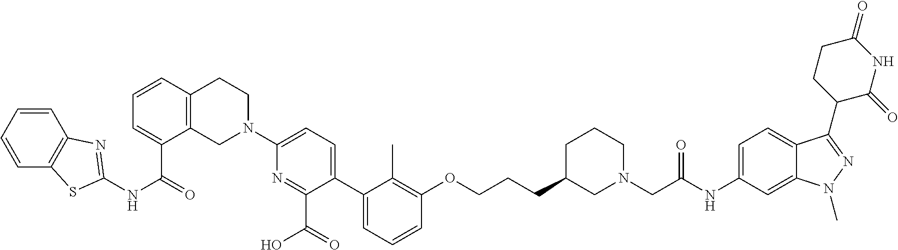 [SMILES: Cc1c(OCCC[C@H]2CCCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-((3R)-1-(2-((3-(2,6-dioxopiperidin-3-yl)-1-methyl-1H-indazol-6-yl)amino)-2-oxoethyl)piperidin-3-yl)propoxy)-2-methylphenyl)picolinic acid

### Step A. Procedure for Preparation of (S)-tert-butyl 3-formylpiperidine-1-carboxylate

A mixture of (S)-tert-butyl 3-(hydroxymethyl)piperidine-1-carboxylate (5.00 g, 23.22 mmol, 1 equiv.) and DMP (10.84 g, 25.55 mmol, 7.91 mL, 1.1 equiv.) in DCM (50 mL) was degassed and purged with N₂ and stirred at 0° C. for 3 hours under N₂ atmosphere. The reaction mixture was diluted with saturated sodium bicarbonate solution (70 mL) and extracted with DCM (70 mL×3). The combined organic layers were washed with brine (70 mL×3), dried over anhydrous sodium sulfate, filtered, and concentrated under reduced pressure to give (S)-tert-butyl 3-formylpiperidine-1-carboxylate (4 g, crude) as a white solid.

### Step B. Procedure for Preparation of (R,E)-tert-butyl 3-(3-ethoxy-3-oxoprop-1-en-1-yl)piperidine-1-carboxylate

A mixture of (S)-tert-butyl 3-formylpiperidine-1-carboxylate (4.00 g, 18.76 mmol, 1 equiv.) in THE (100 mL) was added NaH (0.99 g, 24.75 mmol, 60% purity, 1.32 equiv.) at 0° C., and the mixture was stirred at 0° C. for 60 minutes. Ethyl 2-(diethoxyphosphoryl)acetate (8.41 g, 37.51 mmol, 7.44 mL, 2 equiv.) was added to the mixture, and then the mixture was stirred at 25° C. for 48 hours under N₂ atmosphere. The reaction mixture was quenched by addition water (150 mL) at 0° C. and extracted with EtOAc (200 mL×3). The combined organic layers were washed with brine (200 mL×3), dried over anhydrous sodium sulfate filtered, and concentrated under reduced pressure to give a residue. The residue was purified by flash silica gel chromatography (Eluent of 0˜18% ethyl acetate/petroleum ether) to give (R,E)-tert-butyl 3-(3-ethoxy-3-oxoprop-1-en-1-yl)piperidine-1-carboxylate (3.1 g, 11.0 mmol, 59.1% yield, 99.1% purity) as a colorless oil.

MS (ESI) m/z: 184.1 [M−100+H]⁺

¹H NMR (400 MHz, CD₃Cl) δ=6.84 (dd, J=16.0, 6.8 Hz, 1H), 5.86 (d, J=16.0 Hz, 1H), 4.19 (q, J=7.2 Hz, 2H), 3.93-3.88 (m, 1H), 2.88-2.57 (m, 2H), 2.38-2.26 (m, 1H), 1.92-1.83 (m, 1H), 1.77-1.62 (m, 2H), 1.54-1.48 (m, 1H), 1.46 (s, 9H), 1.42-1.33 (m, 1H), 1.29 (t, J=7.2 Hz, 3H).

### Step C. Procedure for Preparation of (R)-tert-butyl 3-(3-ethoxy-3-oxopropyl)piperidine-1-carboxylate

To a solution of (R,E)-tert-butyl 3-(3-ethoxy-3-oxoprop-1-en-1-yl)piperidine-1-carboxylate (3.16 g, 11.15 mmol, 1 equiv.) in EtOH (30 mL) was added PtO₂ (253.13 mg, 1.11 mmol, 0.1 equiv.) under N₂. The suspension was degassed under vacuum and purged with H₂ several times. The mixture was stirred at 25° C. for 16 hours under H₂ (15 psi) atmosphere. The reaction mixture was filtered and concentrated under reduced pressure to give (R)-tert-butyl 3-(3-ethoxy-3-oxopropyl)piperidine-1-carboxylate (2.1 g, 7.6 mmol, 68.5% yield) as a colorless oil.

¹H NMR (400 MHz, CD₃Cl) δ=4.09 (q, J=7.2 Hz, 2H), 3.88-3.80 (m, 1H), 2.84-2.69 (m, 1H), 2.61-2.37 (m, 1H), 2.30 (t, J=8.0 Hz, 2H), 1.83-1.75 (m, 1H), 1.66-1.45 (m, 4H), 1.42 (s, 9H), 1.41-1.32 (m, 2H), 1.22 (t, J=7.2 Hz, 3H), 1.14-1.02 (m, 1H)

### Step D. Procedure for Preparation of (R)-tert-butyl 3-(3-hydroxypropyl)piperidine-1-carboxylate

A mixture of (R)-tert-butyl 3-(3-ethoxy-3-oxopropyl)piperidine-1-carboxylate (1 g, 3.50 mmol, 1 equiv.) in THE (20 mL) was added LiAlH₄ (106.40 mg, 2.80 mmol, 0.8 equiv.) at 0° C. The mixture was stirred at 0° C. for 10 minutes, and then the mixture was stirred at 0° C. for 18 hours under N₂ atmosphere. The reaction mixture was quenched by addition saturated sodium carbonate solution (0.3 mL) at 0° C., and then diluted with EtOAc (20 mL). The combined organic layers were filtered and concentrated under reduced pressure to give a residue. The residue was purified by flash silica gel chromatography (Eluent of 0˜30% ethyl acetate/petroleum ether) and prep-HPLC to give (R)-tert-butyl 3-(3-hydroxypropyl)piperidine-1-carboxylate (370 mg, 1.4 mmol, 42.2% yield, 97.4% purity) as a colorless oil.

¹H NMR (400 MHz, CD₃C1) 6=4.01-3.83 (m, 2H), 3.65 (t, J=6.4 Hz, 2H), 2.85-2.74 (m, 1H), 2.55-2.43 (m, 1H), 1.90-1.79 (m, 1H), 1.66-1.58 (m, 3H), 1.46 (s, 9H), 1.45-1.36 (m, 2H), 1.36-1.20 (m, 2H), 1.15-1.03 (m, 1H).

SFC Column: Chiralpak IC-3 50×4.6 mm I.D., 3 μm; Mobile phase: Phase A for CO₂, and Phase B for MeOH (0.05% DEA); Gradient elution: B in A from 5% to 40%; Flow rate: 3 mL/min; Detector: PDA; Column Temp: 35C; Back Pressure: 100 Bar.

### Step E. Procedure for Preparation of (R)-tert-butyl 3-(3-(3-bromo-2-methylphenoxy)propyl)piperidine-1-carboxylate

A mixture of (R)-tert-butyl 3-(3-hydroxypropyl)piperidine-1-carboxylate (320 mg, 1.32 mmol, 1 equiv.), 3-bromo-2-methyl-phenol (270.55 mg, 1.45 mmol, 1.1 equiv.), and 2-(tributyl-λ⁵-phosphanylidene)acetonitrile (380.86 mg, 1.58 mmol, 1.2 equiv.) in toluene (10 mL) was degassed and purged with N₂ and stirred at 120° C. for 16 hours under N₂ atmosphere. The reaction mixture was diluted with water (30 mL) and extracted with EtOAc (30 mL×3). The combined organic layers were washed with brine (30 mL×3), dried over anhydrous sodium sulfate, filtered, and concentrated under reduced pressure to give a residue. The residue was purified by reverse-phase HPLC (with 0.1% HCOOH) to give (R)-tert-butyl 3-(3-(3-bromo-2-methylphenoxy)propyl)piperidine-1-carboxylate (300 mg, 727.5 μmol, 55.3% yield, 100% purity) as a yellow oil.

MS (ESI) m/z: 313.8 [M−100+H]⁺

¹H NMR (400 MHz, DMSO-d₆) δ=7.16-7.12 (m, 1H), 7.11-7.05 (m, 1H), 6.95 (d, J=7.6 Hz, 1H), 3.96 (t, J=6.4 Hz, 2H), 3.84-3.62 (m, 2H), 2.84-2.75 (m, 1H), 2.23 (s, 3H), 1.80-1.70 (m, 3H), 1.62-1.52 (m, 1H), 1.37 (s, 9H), 1.35-1.04 (m, 6H).

### Step F. Procedure for Preparation of (R)-3-(3-(3-bromo-2-methylphenoxy)propyl)piperidine

A mixture of (R)-tert-butyl 3-(3-(3-bromo-2-methylphenoxy)propyl)piperidine-1-carboxylate (270 mg, 654.77 μmol, 1 equiv.) in HCl/dioxane (5 mL) and DCM (5 mL) was degassed and purged with N₂ and stirred at 25° C. for 1 hour under N₂ atmosphere. The reaction mixture was concentrated under reduced pressure to give (R)-3-(3-(3-bromo-2-methylphenoxy)propyl)piperidine (260 mg, crude, HCl) as a white solid.

MS (ESI) m/z: 311.9 [M+H]⁺

¹H NMR (400 MHz, DMSO-d₆) δ=7.17-7.13 (m, 1H), 7.12-7.06 (m, 1H), 6.96 (d, J=8.0 Hz, 1H), 3.97 (t, J=6.0 Hz, 2H), 3.39 (s, 3H), 3.24-3.12 (m, 2H), 2.78-2.66 (m, 1H), 2.23 (s, 3H), 1.80-1.72 (m, 4H), 1.46-1.31 (m, 2H), 1.26-1.02 (m, 2H)

### Step G. Procedure for Preparation of (R)-ethyl 2-(3-(3-(3-bromo-2-methylphenoxy)propyl)piperidin-1-yl)acetate

A mixture of (R)-3-(3-(3-bromo-2-methylphenoxy)propyl)piperidine (250 mg, 716.94 μmol, 1 equiv., HCl), ethyl 2-bromoacetate (143.68 mg, 860.33 μmol, 95.15 μL, 1.2 equiv.), and K₂CO₃ (495.43 mg, 3.58 mmol, 5 equiv.) in CH₃CN (4 mL) and DMF (5 mL) was degassed and purged with N₂ and stirred at 70° C. for 1 hour under N₂ atmosphere. The reaction mixture was filtered and concentrated under reduced pressure to give a residue. The residue was purified by reverse-phase HPLC (with 0.1% HCOOH) to give (R)-ethyl 2-(3-(3-(3-bromo-2-methylphenoxy)propyl)piperidin-1-yl)acetate (130 mg, 283.1 μmol, 39.4% yield, 96.9% purity, FA) as a yellow oil.

MS (ESI) m/z: 398.0 [M+H]⁺

¹H NMR (400 MHz, DMSO-d₆,) δ=8.13 (s, 2H), 7.17-7.13 (m, 1H), 7.11-7.06 (m, 1H), 6.95 (d, J=8.0 Hz, 1H), 4.13-4.06 (m, 2H), 3.96 (t, J=6.0 Hz, 2H), 3.34 (s, 2H), 2.93-2.81 (m, 2H), 2.23 (s, 3H), 2.04-1.97 (m, 1H), 1.77-1.69 (m, 3H), 1.67-1.42 (m, 4H), 1.37-1.29 (m, 2H), 1.19 (t, J=7.2 Hz, 3H), 0.93-0.82 (m, 1H)

### Step H. Procedure for Preparation of (R)-tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)piperidin-3-yl)propoxy)-2-methylphenyl)picolinate

(R)-ethyl 2-(3-(3-(3-bromo-2-methylphenoxy)propyl)piperidin-1-yl)acetate (120 mg, 301.26 μmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (221.44 mg, 361.51 μmol, 1.2 equiv.), KF (1.5 M, 602.51 μL, 3 equiv.), and Ad₂nBuP Pd G₃ (cataCXium® A Pd G₃) (21.94 mg, 30.13 μmol, 0.1 equiv.) were taken up into a microwave tube in dioxane (3 mL). The sealed tube was heated at 100° C. for 1 hour under microwave. The reaction mixture was concentrated under reduced pressure to give a residue. The residue was purified by prep-TLC (SiO₂, petroleum ether:ethyl acetate=1:1) to give (R)-tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)piperidin-3-yl)propoxy)-2-methylphenyl)picolinate (200 mg, 233.3 μmol, 77.4% yield, 93.8% purity) as a yellow oil.

MS (ESI) m/z: 804.8 [M+H]⁺

¹H NMR (400 MHz, DMSO-d₆) δ=12.99-12.72 (m, 1H), 8.05-7.98 (m, 1H), 7.77 (d, J=8.0 Hz, 1H), 7.60 (d, J=7.6 Hz, 1H), 7.48-7.41 (m, 3H), 7.39-7.31 (m, 2H), 7.12-7.06 (m, 1H), 6.96-6.88 (m, 2H), 6.57 (d, J=7.6 Hz, 1H), 4.97 (s, 2H), 4.10-3.99 (m, 4H), 3.93 (s, 6H), 3.15 (s, 2H), 3.03 (t, J=5.6 Hz, 2H), 2.80-2.67 (m, 2H), 1.99 (s, 2H), 1.86 (s, 3H), 1.77-1.65 (m, 4H), 1.18-1.15 (m, 4H), 1.00 (s, 9H)

### Step I. Procedure for preparation of (R)-2-(3-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)acetic acid

A mixture of (R)-tert-butyl 6-(8-(benzo[d]thiazol-2-ylcarbamoyl)-3,4-dihydroisoquinolin-2(1H)-yl)-3-(3-(3-(1-(2-ethoxy-2-oxoethyl)piperidin-3-yl)propoxy)-2-methylphenyl)picolinate (190 mg, 236.32 μmol, 1 equiv.) and LiOH H₂O (49.58 mg, 1.18 mmol, 5 equiv.) in THE (4 mL) and H₂O (1 mL) was degassed and purged with N₂ and stirred at 25° C. for 36 hours under N₂ atmosphere. The reaction mixture was diluted with water (20 mL) and extracted with EtOAc (25 mL×3). The combined organic layers were washed with brine (15 mL×3), dried over anhydrous sodium sulfate, filtered and concentrated under reduced pressure to give (R)-2-(3-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)acetic acid (100 mg, 128.8 μmol, 54.5% yield) as a yellow oil.

MS (ESI) m/z: 776.3 [M+H]⁺

¹H NMR (400 MHz, DMSO-d₆,) δ=13.24-12.50 (m, 1H), 8.73-8.37 (m, 1H), 8.09-7.99 (m, 1H), 7.82-7.72 (m, 1H), 7.64-7.57 (m, 1H), 7.53-7.29 (m, 4H), 7.20-7.05 (m, 1H), 7.04-6.83 (m, 2H), 6.50 (d, J=5.2 Hz, 1H), 5.27-4.82 (m, 4H), 4.11-3.94 (m, 6H), 3.91-3.83 (s, 3H), 3.44-3.36 (m, 2H), 3.08-3.00 (m, 2H), 1.99 (s, 2H), 1.91-1.87 (m, 4H), 1.81-1.76 (m, 4H), 1.07 (s, 9H).

### Step J. Procedure for Preparation of tert-butyl 6-(8-(benzo[d]thiazol-2-ylcarbamoyl)-3,4-dihydroisoquinolin-2(1H)-yl)-3-(3-(3-((3R)-1-(2-((3-(2,6-dioxopiperidin-3-yl)-1-methyl-11H-indazol-6-yl)amino)-2-oxoethyl)piperidin-3-yl)propoxy)-2-methylphenyl)picolinate

A mixture of (R)-2-(3-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)acetic acid (90 mg, 115.99 μmol, 1 equiv.), 3-(6-amino-1-methyl-1H-indazol-3-yl)piperidine-2,6-dione (32.95 mg, 127.59 μmol, 1.1 equiv.), and DIEA (44.97 mg, 347.97 μmol, 60.61 μL, 3 equiv.) in DMF (1.2 mL) was degassed and purged with N₂ and stirred at 25° C. for 5 minutes. After 5 minutes, HATU (52.92 mg, 139.19 μmol, 1.2 equiv.) was added, and the mixture was stirred at 25° C. for 1 hour under N₂ atmosphere. The reaction mixture was diluted with water (20 mL) and extracted with EtOAc (25 mL×3). The combined organic layers were washed with brine (15 mL×6), dried over anhydrous sodium sulfate, filtered, and concentrated under reduced pressure to give tert-butyl 6-(8-(benzo[d]thiazol-2-ylcarbamoyl)-3,4-dihydroisoquinolin-2(1H)-yl)-3-(3-(3-((3R)-1-(2-((3-(2,6-dioxopiperidin-3-yl)-1-methyl-1H-indazol-6-yl)amino)-2-oxoethyl)piperidin-3-yl)propoxy)-2-methylphenyl)picolinate (110 mg, crude) as a pink oil.

MS (ESI) m/z: 508.9 [M12+H]⁺

### Step K. Procedure for Preparation of 6-(8-(benzo[d]thiazol-2-ylcarbamoyl)-3,4-dihydroisoquinolin-2(1H)-yl)-3-(3-(3-((3R)-1-(2-((3-(2,6-dioxopiperidin-3-yl)-1-methyl-1H-indazol-6-yl)amino)-2-oxoethyl)piperidin-3-yl)propoxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(3-(3-((3R)-1-(2-((3-(2,6-dioxopiperidin-3-yl)-1-methyl-1H-indazol-6-yl)amino)-2-oxoethyl)piperidin-3-yl)propoxy)-2-methylphenyl)picolinate (100 mg, 98.40 μmol, 1 equiv.) in DCM (0.5 mL) and TFA (0.5 mL) was degassed and purged with N₂ and stirred at 25° C. for 16 hours under N₂ atmosphere. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(3-((3R)-1-(2-((3-(2,6-dioxopiperidin-3-yl)-1-methyl-1H-indazol-6-yl)amino)-2-oxoethyl)piperidin-3-yl)propoxy)-2-methylphenyl)picolinic acid (19.0 mg, 19.8 μmol, 20.1% yield, 100% purity) as a yellow solid.

MS (ESI) m/z: 960.6 [M+H]⁺

¹H NMR (400 MHz, DMSO-d₆,) δ=13.11-12.36 (m, 1H), 10.92-10.84 (m, 1H), 9.85 (s, 1H), 8.15 (s, 1H), 8.06-7.99 (m, 2H), 7.81-7.75 (m, 1H), 7.65-7.58 (m, 2H), 7.50-7.31 (m, 5H), 7.23-7.17 (m, 1H), 7.10-7.03 (m, 1H), 6.97-6.91 (m, 1H), 6.88-6.82 (m, 1H), 6.64-6.59 (m, 1H), 4.97 (s, 2H), 4.34-4.27 (m, 1H), 4.00-3.91 (m, 3H), 3.90 (s, 3H), 3.21-3.08 (m, 4H), 3.05-2.99 (m, 2H), 2.89-2.78 (m, 2H), 2.67-2.58 (m, 2H), 2.37-2.28 (m, 2H), 2.20-2.11 (m, 2H), 1.88 (s, 3H), 1.82-1.67 (m, 4H), 1.66-1.58 (m, 2H), 1.41-1.32 (m, 2H).

### Example 161. Preparation of Compound 209a

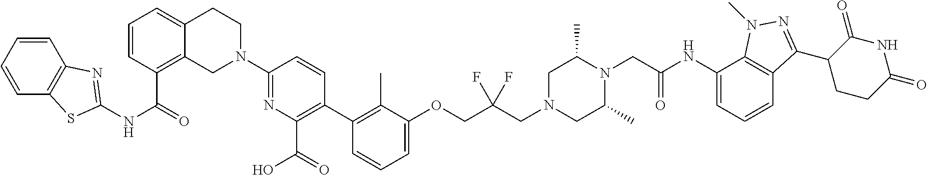 [SMILES: Cc1c(OCC(F)(F)CN2C[C@@H](C)N(CC(=O)Nc3cccc4c(C5CCC(=O)NC5=O)nn(C)c34)[C@@H](C)C2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[(3R,5S)-4-[2-[[3-(2,6-dioxo-3-piperidyl)-1-methyl-indazol-7-yl]amino]-2-oxo-ethyl]-3,5-dimethyl-piperazin-1-yl]-2,2-difluoro-propoxy]-2-methyl-phenyl]pyridine-2-carboxylic acid

### Step A. Procedure for Preparation of tert-butyl 6-[8-(1,3-benzothiazol-2-ylcarbamoyl)-3,4-dihydro-1H-isoquinolin-2-yl]-3-[3-[3-[(3R,5S)-4-[2-[[3-(2,6-dioxo-3-piperidyl)-1-methyl-indazol-7-yl]amino]-2-oxo-ethyl]-3,5-dimethyl-piperazin-1-yl]-2,2-difluoro-propoxy]-2-methyl-phenyl]pyridine-2-carboxylate

A solution of 2-[(2R,6S)-4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-2,2-difluoro-propyl]-2,6-dimethyl-piperazin-1-yl]acetic acid (100 mg, 118.91 μmol, 1.0 equiv.), 3-(7-amino-1-methyl-indazol-3-yl)piperidine-2,6-dione (46.07 mg, 178.36 μmol, 1.5 equiv.), and EDCI (34.19 mg, 178.36 μmol, 1.5 equiv.) in pyridine (1.0 mL) was stirred at 25° C. for 5 hours. The mixture was quenched by water (10 mL) and filtered. The filter cake was concentrated under vacuum to give tert-butyl 6-[8-(1,3-benzothiazol-2-ylcarbamoyl)-3,4-dihydro-1H-isoquinolin-2-yl]-3-[3-[3-[(3R,5S)-4-[2-[[3-(2,6-dioxo-3-piperidyl)-1-methyl-indazol-7-yl]amino]-2-oxo-ethyl]-3,5-dimethyl-piperazin-1-yl]-2,2-difluoro-propoxy]-2-methyl-phenyl]pyridine-2-carboxylate (135 mg, crude) as a white solid.

MS (ESI) m/z: 1082.8 [M+H]⁺.

### Step B. Procedure for Preparation of 6-[8-(1,3-benzothiazol-2-ylcarbamoyl)-3,4-dihydro-1H-isoquinolin-2-yl]-3-[3-[3-[(3R,5S)-4-[2-[[3-(2,6-dioxo-3-piperidyl)-1-methyl-indazol-7-yl]amino]-2-oxo-ethyl]-3,5-dimethyl-piperazin-1-yl]-2,2-difluoro-propoxy]-2-methyl-phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[3-[(3R,5S)-4-[2-[[3-(2,6-dioxo-3-piperidyl)-1-methyl-indazol-7-yl]amino]-2-oxo-ethyl]-3,5-dimethyl-piperazin-1-yl]-2,2-difluoro-propoxy]-2-methyl-phenyl]pyridine-2-carboxylate (130 mg, 120.23 μmol, 1.0 equiv.) in TFA (1 mL) and DCM (1 mL) was stirred at 25° C. for 16 hours. The mixture was concentrated to remove DCM. The residue was purified by prep-HPLC to give 6-[8-(1,3-benzothiazol-2-ylcarbamoyl)-3,4-dihydro-1H-isoquinolin-2-yl]-3-[3-[3-[(3R,5S)-4-[2-[[3-(2,6-dioxo-3-piperidyl)-1-methyl-indazol-7-yl]amino]-2-oxo-ethyl]-3, 5-dimethyl-piperazin-1-yl]-2,2-difluoro-propoxy]-2-methyl-phenyl]pyridine-2-carboxylic acid (61.9 mg, 58.1 μmol, 48.3% yield, 96.1% purity) as a white solid.

MS (ESI) m/z: 513.0 [M12+H]⁺.

¹H NMR (400 MHz, DMSO-d₆) δ=12.87 (s, 1H), 10.90 (s, 1H), 9.79 (s, 1H), 8.14 (s, 1H), 8.03 (d, J=7.6 Hz, 1H), 7.79 (d, J=8.0 Hz, 1H), 7.62 (d, J=7.6 Hz, 1H), 7.57 (d, J=8.0 Hz, 1H), 7.49-7.42 (m, 3H), 7.39-7.32 (m, 2H), 7.30 (d, J=7.2 Hz, 1H), 7.16-7.11 (m, 1H), 7.07 (t, J=8.0 Hz, 1H), 6.98 (dd, J=6.0, 8.4 Hz, 2H), 6.71 (d, J=7.6 Hz, 1H), 5.06-4.93 (m, 2H), 4.40-4.31 (m, 3H), 4.10 (s, 3H), 3.92 (t, J=6.0 Hz, 2H), 3.03 (t, J=5.6 Hz, 2H), 2.94 (s, 1H), 2.92-2.85 (m, 2H), 2.78 (s, 1H), 2.75 (d, J=10.8 Hz, 2H), 2.70-2.66 (m, 2H), 2.62 (t, J=5.2 Hz, 1H), 2.39-2.32 (m, 1H), 2.24-2.15 (m, 3H), 1.95 (s, 1H), 1.93 (s, 3H), 1.04 (d, J=6.0 Hz, 6H).

¹⁹F NMR (400 MHz, DMSO-d₆) δ=106.40.

### Example 162. Preparation of Compound 211a

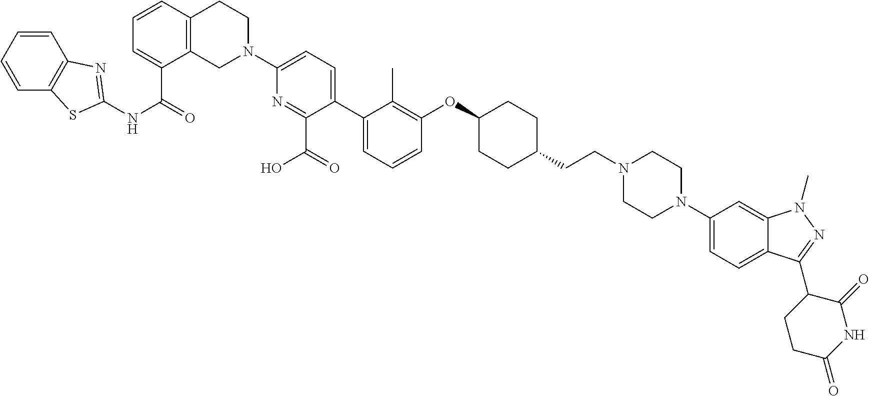 [SMILES: Cc1c(O[C@H]2CC[C@H](CCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-(2-(4-(3-(2,6-dioxopiperidin-3-yl)-1-methyl-1H-indazol-6-yl)piperazin-1-yl)ethyl)cyclohexyl)oxy)-2-methylphenyl)picolinic acid

### Step A. Procedure for Preparation of methyl 4-(3-bromo-2-methyl-phenoxy)cyclohexane carboxylate

To a solution of methyl 4-hydroxycyclohexanecarboxylate (16 g, 101.14 mmol, 1.0 equiv.) and 3-bromo-2-methyl-phenol (20.81 g, 111.26 mmol, 1.1 equiv.) in Tol. (500 mL) was added 2-(tributyl-λ5-phosphanylidene)acetonitrile (29.29 g, 121.37 mmol, 1.2 equiv.). The mixture was stirred at 120° C. for 12 hours. The reaction mixture was concentrated under reduced pressure to give the compound methyl 4-(3-bromo-2-methyl-phenoxy)cyclohexane carboxylate (125 g, crude) was obtained as a black oil, which was used in the next step without further purification.

### Step B. Procedure for Preparation of [4-(3-bromo-2-methyl-phenoxy)cyclohexyl]methanol

A mixture of methyl 4-(3-bromo-2-methyl-phenoxy)cyclohexane carboxylate (28 g, 34.23 mmol, 40% purity, 1.0 equiv.) in THE (300 mL) was slowly added LAH (1.36 g, 35.94 mmol, 1.05 equiv.) at 0° C. then the mixture was stirred at 0° C. for 1 hour. The mixture was quenched by 2.5 g Na₂SO₄·10 H₂O. The solution was poured into ice-water (250 mL) slowly and the pH was adjusted to pH 4-5 with 1 M HCl, extracted with EtOAc 600 mL (200 mL×3), washed with brine 300 mL (150 mL×2), dried over by sodium sulfate, filtered and concentrated under reduced pressure. The residue was purified by flash silica gel chromatography (Eluent of 0-18% ethyl acetate/petroleum ether). The compound [4-(3-bromo-2-methyl-phenoxy)cyclohexyl]methanol (10 g, 32.3 mmol, 47.2% yield, 96.7% purity) was obtained as a brown oil.

### Step C. Procedure for Preparation of 4-(3-bromo-2-methyl-phenoxy)cyclohexane carbaldehyde

To a solution of DMSO (5.22 g, 66.84 mmol, 5.22 mL, 4 equiv.) in DCM (200 mL) was added the mixture of (COCl)₂ (4.24 g, 33.42 mmol, 2.93 mL, 2 equiv.) under −78° C. and stirred for 1 hour. And [4-(3-bromo-2-methyl-phenoxy)cyclohexyl]methanol (5 g, 16.71 mmol, 1.0 equiv.) was added into the mixture and stirred for 1 hour. TEA (10.15 g, 100.27 mmol, 13.96 mL, 6.0 equiv.) was added into the mixture and stirred at −70° C. for 1 hour. The reaction mixture was diluted with H₂O (200) mL and extracted with DCM 60 mL (30 mL×2). Dried over [Na₂SO₄] and concentrated under reduced pressure to give 4-(3-bromo-2-methyl-phenoxy)cyclohexane carbaldehyde (17 g, crude) as a yellow oil, which was used into nest step without further purification.

### Step D. Procedure for Preparation of 1-bromo-3-[4-[(E)-2-methoxyvinyl]cyclohexoxy]-2-methyl-benzene

A mixture of methoxymethyl (triphenyl)phosphonium; bromide (1.69 g, 4.37 mmol, 1.30 equiv.) in THE (10 mL) was degassed and purged with N₂. The mixture was slowly added LiHMDS (1 M, 4.37 mL, 1.30 equiv.) at 0° C. and stirred for 2 hours. Then, 4-(3-bromo-2-methyl-phenoxy)cyclohexane carbaldehyde (1 g, 3.36 mmol, 1.00 equiv.) was added to the mixture and stirred at 25° C. for 13 hours under N₂ atmosphere. The reaction mixture was quenched by addition saturated NH₄Cl (50 mL), and then extracted with ethyl acetate (40 mL×3). The combined organic layers were washed with brine 90 mL (30 mL×3), dried over Na₂SO₄, filtered and concentrated under reduced pressure to give a residue. The residue was purified by column chromatography (SiO₂, petroleum ether/ethyl acetate=1/0 to 10/1) to give 1-bromo-3-[4-[(E)-2-methoxyvinyl]cyclohexoxy]-2-methyl-benzene (0.6 g, 1.8 mmol, 54.8% yield) as a white solid.

### Step E. Procedure for Preparation of 2-[4-(3-bromo-2-methyl-phenoxy)cyclohexyl]acetaldehyde

A mixture of 1-bromo-3-[4-[(E)-2-methoxyvinyl]cyclohexoxy]-2-methyl-benzene (0.6 g, 1.84 mmol, 1 equiv.), HCl (2 M, 2.77 mL, 3 equiv.) in THE (2 mL) was stirred at 80° C. for 12 hours under N₂ atmosphere. The reaction mixture was diluted with water 10 mL and extracted with ethyl acetate (20 mL×3). The combined organic layers were washed with brine 30 mL (10 mL×3), dried over Na₂SO₄, filtered and concentrated under reduced pressure to give a residue. The residue was purified by column chromatography (SiO₂, petroleum ether/ethyl acetate=1/0 to 10/1) to give 2-[4-(3-bromo-2-methyl-phenoxy)cyclohexyl]acetaldehyde (0.3 g, 889.7 μmol, 48.3% yield, 92.3% purity) was obtained as a colorless oil.

### Step F. Procedure for Preparation of 2-[4-(3-bromo-2-methyl-phenoxy)cyclohexyl]ethanol

A mixture of 2-[4-(3-bromo-2-methyl-phenoxy)cyclohexyl]acetaldehyde (200.00 mg, 642.65 μmol, 1.00 equiv.), LiAlH₄ (24.39 mg, 642.65 μmol, 1.00 equiv.) in THF (5 mL) was degassed and purged with N₂ and stirred at 0° C. for 1 hour under N₂ atmosphere. The reaction mixture was quenched by addition water 5 mL, and then extracted with ethyl acetate (10 mL×2). The combined organic layers were washed with brine 20 mL (10 mL×2), dried over Na₂SO₄, filtered and concentrated under reduced pressure to give a residue. The residue was further separated by SFC. The compound 2-[4-(3-bromo-2-methyl-phenoxy)cyclohexyl]ethanol (60 mg, 191.5 μmol, 29.8% yield) was obtained as a white solid.

¹H NMR (400 MHz, CH₃OD) δ=7.05 (d, J=8.0 Hz, 1H), 6.95 (t, J=8.0 Hz, 1H), 6.85 (d, J=8.0 Hz, 1H), 4.20-4.06 (m, 1H), 3.62-3.52 (m, 2H), 2.21 (s, 3H), 2.08 (d, J=12.4 Hz, 2H), 1.81 (d, J=13.2 Hz, 2H), 1.46-1.33 (m, 5H), 1.12-0.99 (m, 2H).

### Step G. Procedure for Preparation of 2-[4-(3-bromo-2-methyl-phenoxy)cyclohexyl]acetaldehyde

To a solution of oxalyl dichloride (50.91 mg, 401.07 μmol, 35.11 μL, 2 equiv.) in DCM (3 mL) was added the mixture of DMSO (62.67 mg, 802.13 μmol, 62.67 μL, 4 equiv.) under −70° C. and stirred for 1 h. And 2-[4-(3-bromo-2-methyl-phenoxy)cyclohexyl]ethanol (62.81 mg, 200.53 μmol, 1 equiv.) was added into the mixture and stirred for 1 h. TEA (121.75 mg, 1.20 mmol, 167.47 μL, 6 equiv.) was added into the mixture and stirred at −70° C. for 1 h. The reaction mixture was filtered and concentrated under reduced pressure to remove solvent. The residue was diluted with ethyl acetate (200 mL) and extracted with H₂O (30 mL×2). Dried over Na₂SO₄, and concentrated under reduced pressure to give a residue. The compound 2-[4-(3-bromo-2-methyl-phenoxy)cyclohexyl]acetaldehyde (50 mg, 160.66 μmol, 80.1% yield) was obtained as a colorless oil.

### Step H. Procedure for Preparation of tert-butyl 6-[8-(1,3-benzothiazol-2-ylcarbamoyl)-3,4-dihydro-1H-isoquinolin-2-yl]-3-[2-methyl-3-[4-(2-oxoethyl)cyclohexoxy]phenyl]pyridine-2-carboxylate

A mixture of 2-[4-(3-bromo-2-methyl-phenoxy)cyclohexyl]acetaldehyde (50 mg, 160.66 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (108.25 mg, 176.73 μmol, 1.1 equiv.), Ad₂nBuP Pd G₃ (11.70 mg, 16.07 μmol, 0.1 equiv.), KF (1.5 M, 321.32 μL, 3 equiv.) in dioxane (2 mL) was degassed and purged with N₂ three times, and then the mixture was stirred at 100° C. for 1 h under N₂ atmosphere in microwave. The reaction mixture was concentrated under reduced pressure to give a residue. The residue was purified by prep-TLC (SiO₂, petroleum ether: ethyl acetate=2:1). The compound tert-butyl 6-[8-(1,3-benzothiazol-2-ylcarbamoyl)-3,4-dihydro-1H-isoquinolin-2-yl]-3-[2-methyl-3-[4-(2-oxoethyl)cyclohexoxy]phenyl]pyridine-2-carboxylate (130 mg, 117.6 μmol, 73.2% yield, 64.9% purity) was obtained as a yellow solid.

MS (ESI) m/z: 717.5 [M+H]⁺.

### Step I. Procedure for preparation of tert-butyl 6-[8-(1,3-benzothiazol-2-ylcarbamoyl)-3,4-dihydro-1H-isoquinolin-2-yl]-3-[3-[4-[2-[4-[3-(2,6-dioxo-3-piperidyl)-1-methyl-indazol-6-yl]piperazin-1-yl]ethyl]cyclohexoxy]-2-methyl-phenyl]pyridine-2-carboxylate

A mixture of 3-(1-methyl-6-piperazin-1-yl-indazol-3-yl)piperidine-2,6-dione (27.40 mg, 83.70 μmol, 1 equiv.), NaBH(OAc)₃ (26.61 mg, 125.54 μmol, 1.5 equiv.), AcOH (1.01 mg, 16.74 μmol, 9.57e-1 μL, 0.2 equiv.) in DCM (2 mL) was slowly added tert-butyl 6-[8-(1,3-benzothiazol-2-ylcarbamoyl)-3,4-dihydro-1H-isoquinolin-2-yl]-3-[2-methyl-3-[4-(2-oxoethyl)cyclohexoxy]phenyl]pyridine-2-carboxylate (60 mg, 83.70 μmol, 1 equiv.) at 0° C., the mixture was stirred at 25° C. for 1 h under N₂ atmosphere. The reaction mixture was filtered and concentrated under reduced pressure to give a residue. The compound tert-butyl 6-[8-(1,3-benzothiazol-2-ylcarbamoyl)-3,4-dihydro-1H-isoquinolin-2-yl]-3-[3-[4-[2-[4-[3-(2,6-dioxo-3-piperidyl)-1-methyl-indazol-6-yl]piperazin-1-yl]ethyl]cyclohexoxy]-2-methyl-phenyl]pyridine-2-carboxylate (65 mg, crude) was obtained as a yellow solid.

### Step J. Procedure for Preparation of 6-(8-(benzo[d]thiazol-2-ylcarbamoyl)-3,4-dihydroisoquinolin-2(1H)-yl)-3-(3-(((1r,4r)-4-(2-(4-(3-(2,6-dioxopiperidin-3-yl)-1-methyl-1H-indazol-6-yl)piperazin-1-yl)ethyl)cyclohexyl)oxy)-2-methylphenyl)picolinic acid

A mixture of tert-butyl 6-[8-(1,3-benzothiazol-2-ylcarbamoyl)-3,4-dihydro-1H-isoquinolin-2-yl]-3-[3-[4-[2-[4-[3-(2,6-dioxo-3-piperidyl)-1-methyl-indazol-6-yl]piperazin-1-yl]ethyl]cyclohexoxy]-2-methyl-phenyl]pyridine-2-carboxylate (62 mg, 60.30 μmol, 1 equiv.) in TFA (0.5 mL) and DCM (0.5 mL) was stirred at 40° C. for 1 h under N₂ atmosphere. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1r,4r)-4-(2-(4-(3-(2,6-dioxopiperidin-3-yl)-1-methyl-1H-indazol-6-yl)piperazin-1-yl)ethyl)cyclohexyl)oxy)-2-methylphenyl)picolinic acid (24.1 mg, 23.8 μmol, 39.5% yield, 95.9% purity) as a yellow solid.

MS (ESI) m/z: 972.7 [M+H]⁺.

¹H NMR (400 MHz, DMSO-d₆) δ=12.87 (d, J=4.8 Hz, 1H), 12.76-12.28 (m, 1H), 10.86 (s, 1H), 8.04 (d, J=7.2 Hz, 1H), 7.80 (d, J=8.0 Hz, 1H), 7.63 (d, J=7.2 Hz, 1H), 7.57 (d, J=9.2 Hz, 1H), 7.45 (d, J=8.4 Hz, 3H), 7.37 (d, J=10.0 Hz, 2H), 7.09 (s, 1H), 6.99 (s, 4H), 6.63 (d, J=7.2 Hz, 1H), 4.99 (s, 2H), 4.29 (dd, J=5.2, 9.2 Hz, 1H), 4.24 (s, 1H), 3.93 (s, 3H), 3.66-3.60 (m, 2H), 3.24-3.16 (m, 4H), 3.04 (d, J=6.0 Hz, 4H), 2.62 (s, 2H), 2.34 (s, 2H), 2.21-2.06 (m, 4H), 1.88 (s, 3H), 1.85-1.79 (m, 2H), 1.66-1.59 (m, 2H), 1.38 (d, J=1.6 Hz, 4H), 1.24 (s, 1H), 1.17 (s, 2H)

### Example 163. Preparation of Compound 214a

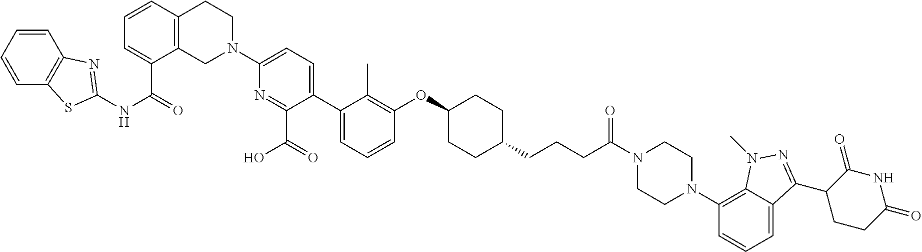 [SMILES: Cc1c(O[C@H]2CC[C@H](CCCC(=O)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-7-yl)piperazin-1-yl)-4-oxobutyl)cyclohexyl)oxy)-2-methylphenyl)picolinic acid

### Step A. Procedure for Preparation of 4-((1r,4s)-4-(3-bromo-2-methylphenoxy)cyclohexyl)butanoic acid

A mixture of 4-((1r,4s)-4-(3-bromo-2-methylphenoxy)cyclohexyl)butanal (500 mg, 1.47 mmol, 1 equiv.), NaH₂PO4 (529.11 mg, 4.41 mmol, 3 equiv.), sodium chlorite (398.85 mg, 4.41 mmol, 3 equiv.), and 2-methylbut-2-ene (618.55 mg, 8.82 mmol, 934.36 μL, 6 equiv.) in t-BuOH (6 mL), THE (2 mL), and H₂O (1 mL) was stirred at 25° C. for 12 hours under N₂ atmosphere. The reaction was diluted with water (50 mL) and extracted with DCM (80 mL×2). The combined organic layers were washed with brine (60 mL×1), dried over Na₂SO₄, filtered, and concentrated under reduced pressure to give a residue. The residue was purified by column chromatography (SiO₂, petroleum ether/ethyl acetate=20/1 to 8/1) to give 4-((1r,4s)-4-(3-bromo-2-methylphenoxy)cyclohexyl)butanoic acid (400 mg, 1.1 mmol, 76.5% yield) as a white solid.

¹H NMR (400 MHz, DMSO-d₆) δ=11.98 (s, 1H), 7.17-7.11 (m, 1H), 7.10-6.99 (m, 2H), 4.30-4.16 (m, 1H), 2.21 (s, 3H), 2.20-2.17 (m, 1H), 2.04 (d, J=11.6 Hz, 2H), 1.76 (d, J=11.6 Hz, 2H), 1.56-1.47 (m, 2H), 1.42-1.33 (m, 2H), 1.31-1.15 (m, 4H), 1.09-0.98 (m, 2H)

### Step B. Procedure for Preparation of 4-((1r,4s)-4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)cyclohexyl)butanoic acid

A mixture of tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (600 mg, 979.52 μmol, 1 equiv.), 4-((1r,4s)-4-(3-bromo-2-methylphenoxy)cyclohexyl)butanoic acid (382.79 mg, 1.08 mmol, 1.1 equiv.), [2-(2-aminophenyl)phenyl]palladium(1⁺); bis(1-adamantyl)-butyl-phosphane; methanesulfonate (71.34 mg, 97.95 μmol, 0.1 equiv.), and KF (170.73 mg, 2.94 mmol, 68.84 μL, 3 equiv.) in dioxane (6 mL) and H₂O (2 mL) was degassed and purged with N₂ and stirred at 100° C. for 12 hours under N₂ atmosphere. The reaction mixture was concentrated under reduced pressure to give a residue. The residue was purified by column chromatography (SiO₂, dichloromethane:methanol=20/1 to 10/1) to give 4-((1r,4s)-4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)cyclohexyl)butanoic acid (400 mg, 525.6 μmol, 53.6% yield) as a yellow solid MS (ESI) m/z: 761.8 [M+H]⁺.

### Step C. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-7-yl)piperazin-1-yl)-4-oxobutyl)cyclohexyl)oxy)-2-methylphenyl)picolinate

To a solution of 4-((1r,4s)-4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)cyclohexyl)butanoic acid (100 mg, 131.42 μmol, 1 equiv.) in pyridine (1 mL) was added EDCI (32.75 mg, 170.84 μmol, 1.3 equiv.) and 3-(1-methyl-7-piperazin-1-yl-indazol-3-yl)piperidine-2,6-dione (51.63 mg, 157.70 μmol, 1.2 equiv.). The mixture was stirred at 60° C. for 12 hours. The reaction was diluted with water (20 mL) and extracted with DCM (20 mL×2). The combined organic layers were washed with brine (30 mL×1), dried over Na₂SO₄, filtered, and concentrated under reduced pressure to give a residue. The residue was purified by prep-TLC to give tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-7-yl)piperazin-1-yl)-4-oxobutyl)cyclohexyl)oxy)-2-methylphenyl)picolinate (60 mg, 56.0 μmol, 42.6% yield) as a yellow solid.

MS (ESI) m/z: 1170.8 [M+H]⁺.

### Step D. Procedure for Preparation of 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-7-yl)piperazin-1-yl)-4-oxobutyl)cyclohexyl)oxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-7-yl)piperazin-1-yl)-4-oxobutyl)cyclohexyl)oxy)-2-methylphenyl)picolinate (60 mg, 56.06 μmol, 1 equiv.) in DCM (0.5 mL) was added TFA (770.00 mg, 6.75 mmol, 0.5 mL, 120.47 equiv.). The mixture was stirred at 40° C. for 1 hour. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-7-yl)piperazin-1-yl)-4-oxobutyl)cyclohexyl)oxy)-2-methylphenyl)picolinic acid (15.3 mg, 14.8 μmol, 26.5% yield, 97.8% purity) as a yellow solid.

MS (ESI) m/z: 1014.9 [M+H]⁺.

¹H NMR (400 MHz, DMSO-d₆) δ=12.87 (s, 1H), 12.72-12.45 (m, 1H), 10.89 (s, 1H), 8.04 (d, J=8.0 Hz, 1H), 7.80 (d, J=8.4 Hz, 1H), 7.63 (d, J=7.2 Hz, 1H), 7.48-7.42 (m, 4H), 7.40-7.33 (m, 2H), 7.08-7.02 (m, 3H), 6.98-6.92 (m, 2H), 6.62 (d, J=7.6 Hz, 1H), 4.98 (s, 2H), 4.53-4.43 (m, 1H), 4.37-4.33 (m, 1H), 4.28 (s, 3H), 4.24-4.17 (m, 1H), 3.92 (t, J=5.6 Hz, 2H), 3.24-3.17 (m, 2H), 3.03 (t, J=5.2 Hz, 2H), 2.68-2.61 (m, 4H), 2.40-2.30 (m, 4H), 2.24-2.12 (m, 2H), 2.11-2.05 (m, 2H), 2.00-1.99 (m, 1H), 1.88 (s, 3H), 1.80 (d, J=12.8 Hz, 2H), 1.59-1.53 (m, 2H), 1.41-1.34 (m, 2H), 1.30-1.20 (m, 4H), 1.12-1.02 (m, 2H)

### Example 164. Preparation of Compound 215a

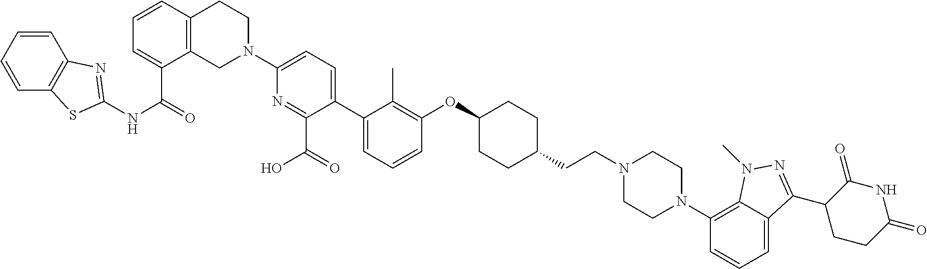 [SMILES: Cc1c(O[C@H]2CC[C@H](CCN3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-(2-(4-(3-(2,6-dioxopiperidin-3-yl)-1-methyl-1H-indazol-7-yl)piperazin-1-yl)ethyl)cyclohexyl)oxy)-2-methylphenyl)picolinic acid

### Step A. Procedure for Preparation of tert-butyl 6-[8-(1,3-benzothiazol-2-ylcarbamoyl)-3,4-dihydro-1H-isoquinolin-2-yl]-3-[3-[4-[2-[4-[3-(2,6-dioxo-3-piperidyl)-1-methyl-indazol-7-yl]piperazin-1-yl]ethyl]cyclohexoxy]-2-methyl-phenyl]pyridine-2-carboxylate

To a mixture of 3-(1-methyl-7-piperazin-1-yl-indazol-3-yl)piperidine-2,6-dione (27.40 mg, 83.70 μmol, 1 equiv.), NaBH(OAc)₃ (26.61 mg, 125.54 μmol, 1.5 equiv.), and AcOH (5.03 mg, 83.70 μmol, 4.79 μL, 0.1 equiv.) in DCM (2 mL) was added tert-butyl 6-[8-(1,3-benzothiazol-2-ylcarbamoyl)-3,4-dihydro-1H-isoquinolin-2-yl]-3-[2-methyl-3-[4-(2-oxoethyl)cyclohexoxy]phenyl]pyridine-2-carboxylate (60 mg, 83.70 μmol, 1 equiv.) at 0° C. The mixture was degassed and purged with N₂ and stirred at 25° C. for 1 hour under N₂ atmosphere. The reaction mixture was filtered and concentrated under reduced pressure to give tert-butyl 6-[8-(1,3-benzothiazol-2-ylcarbamoyl)-3,4-dihydro-1H-isoquinolin-2-yl]-3-[3-[4-[2-[4-[3-(2,6-dioxo-3-piperidyl)-1-methyl-indazol-7-yl]piperazin-1-yl]ethyl]cyclohexoxy]-2-methyl-phenyl]pyridine-2-carboxylate (62 mg, crude) as a yellow solid.

MS (ESI) m/z: 514.9 [M+2H/2]⁺

### Step B. Procedure for Preparation of 6-(8-(benzo[d]thiazol-2-ylcarbamoyl)-3,4-dihydroisoquinolin-2(1H)-yl)-3-(3-(((1r,4r)-4-(2-(4-(3-(2,6-dioxopiperidin-3-yl)-1-methyl-1H-indazol-7-yl)piperazin-1-yl)ethyl)cyclohexyl)oxy)-2-methylphenyl)picolinic acid

A mixture of tert-butyl 6-[8-(1,3-benzothiazol-2-ylcarbamoyl)-3,4-dihydro-1H-isoquinolin-2-yl]-3-[3-[4-[2-[4-[3-(2,6-dioxo-3-piperidyl)-1-methyl-indazol-7-yl]piperazin-1-yl]ethyl]cyclohexoxy]-2-methyl-phenyl]pyridine-2-carboxylate (60 mg, 58.35 μmol, 1 equiv.) in TFA (0.5 mL) and DCM (1.5 mL) was degassed and purged with N₂ and stirred at 25° C. for 25 hours under N₂ atmosphere. The reaction mixture was concentrated under reduced pressure to give a residue. The residue was purified by prep-HPLC. The compound 6-(8-(benzo[d]thiazol-2-ylcarbamoyl)-3,4-dihydroisoquinolin-2(1H)-yl)-3-(3-(((1r,4r)-4-(2-(4-(3-(2,6-dioxopiperidin-3-yl)-1-methyl-1H-indazol-7-yl)piperazin-1-yl)ethyl)cyclohexyl)oxy)-2-methylphenyl)picolinic acid (23.2 mg, 22.7 μmol, 38.9% yield, 95.0% purity) was obtained as a yellow solid.

MS (ESI) m/z: 972.6 [M+H]⁺

### Example 165. Preparation of Compound 222

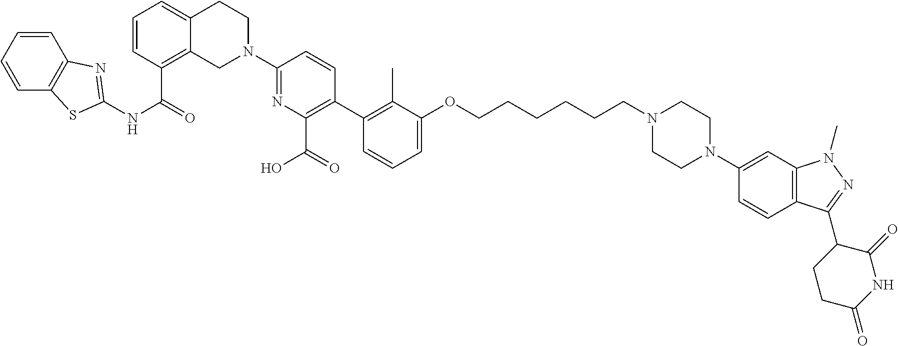 [SMILES: Cc1c(OCCCCCCN2CCN(c3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-((6-(4-(3-(2,6-dioxopiperidin-3-yl)-1-methyl-1H-indazol-6-yl)piperazin-1-yl)hexyl)oxy)-2-methylphenyl)picolinic acid

### Step A. Procedure for Preparation of methyl 6-(3-bromo-2-methylphenoxy)hexanoate

To a solution of 6-(3-bromo-2-methylphenoxy)hexan-1-ol (2 g, 10.69 mmol, 1 equiv.) in CH₃CN (20 mL) was added K₂CO₃ (4.43 g, 32.08 mmol, 3 equiv.) and methyl 6-bromohexanoate (2.46 g, 11.76 mmol, 1.1 equiv.). The mixture was stirred at 60° C. for 2 hours. After cooling to room temperature, ethyl acetate (50 mL) and water (50 mL) were added, and layers were separated. The aqueous phase was extracted with organic layers and washed with ethyl acetate (30 mL×2). The combined organic extracts were dried over anhydrous sodium sulfate, filtered, and concentrated under vacuum. The residue was purified by flash silica gel chromatography (Eluent of 0˜2% ethyl acetate/petroleum ether) to give methyl 6-(3-bromo-2-methylphenoxy)hexanoate (3 g, 9.5 mmol, 89.0% yield) as a colorless oil.

¹H NMR (400 MHz, CDCl₃) δ=7.14 (d, J=8.0 Hz, 1H), 6.99 (t, J=8.0 Hz, 1H), 6.75 (d, J=8.4 Hz, 1H), 3.95 (t, J=6.4 Hz, 2H), 3.68 (s, 3H), 2.36 (t, J=7.6 Hz, 2H), 2.31 (s, 3H), 1.85-1.80 (m, 2H), 1.75-1.69 (m, 2H), 1.55-1.50 (m, 2H)

### Step B. Procedure for Preparation of 6-(3-bromo-2-methylphenoxy)hexan-1-ol

To a solution of methyl 6-(3-bromo-2-methylphenoxy)hexanoate (1.5 g, 4.76 mmol, 1 equiv.) in THE (15 mL) was added LiAlH₄ (180.60 mg, 4.76 mmol, 1 equiv.) at 0° C. The mixture was stirred at 0° C. for 2 hours. The reaction mixture was quenched by addition of water (0.2 mL) and 15% NaOH (0.2 mL) and water (0.6 mL). The combined mixture was treated with Na₂SO₄, filtered, and concentrated under reduced pressure to give 6-(3-bromo-2-methylphenoxy)hexan-1-ol (1.2 g, 4.1 mmol, 87.8% yield) as a yellow oil. The 6-(3-bromo-2-methylphenoxy)hexan-1-ol residue was further purified by prep-HPLC to give 6-(3-bromo-2-methylphenoxy)hexan-1-ol (501.28 mg, 1.7 mmol, 66.2% yield, 99.3% purity) as a yellow oil.

MS (ESI) m/z: 288.8 [M+H]⁺

¹H NMR (400 MHz, DMSO-d₆) δ=7.16-7.13 (m, 1H), 7.11-7.06 (m, 1H), 6.96 (d, J=8.0 Hz, 1H), 4.33 (t, J=5.2 Hz, 1H), 3.97 (t, J=6.4 Hz, 2H), 3.43-3.36 (m, 2H), 2.23 (s, 3H), 1.79-1.67 (m, 2H), 1.46-1.40 (m, 4H), 1.39-1.30 (m, 2H)

### Step D. Procedure for Preparation of 6-(3-bromo-2-methylphenoxy)hexanal

To a solution of oxalyl dichloride (1.06 g, 8.36 mmol, 731.51 uL, 2 equiv.) in DCM (2 mL) was dropwise added a solution of DMSO (1.31 g, 16.71 mmol, 1.31 mL, 4 equiv.) in DCM (2 mL) at −70° C. under N₂ atmosphere. The mixture was stirred at −70° C. for 1 hour. Then 6-(3-bromo-2-methylphenoxy)hexan-1-ol (1.2 g, 4.18 mmol, 1 equiv.) in DCM (10 mL) was added dropwise at −70° C. The solution was stirred for 1 hour at −70° C. Then TEA (2.54 g, 25.07 mmol, 3.49 mL, 6 equiv.) was added to the solution. The solution was stirred at −70° C. for 0.5 hour under N₂ atmosphere. Water (50 mL) was added, and layers were separated. The aqueous phase was extracted with DCM 60 mL (30 mL×2), dried over anhydrous sodium sulfate, filtered, and concentrated under vacuum to give 6-(3-bromo-2-methylphenoxy)hexanal (1.0 g, 3.5 mmol, 83.9% yield) as a yellow oil.

¹H NMR (400 MHz, DMSO-d₆) δ=9.72-9.61 (m, 1H), 7.17-7.12 (m, 1H), 7.11-7.06 (m, 1H), 6.96 (d, J=8.0 Hz, 1H), 3.97 (t, J=6.4 Hz, 2H), 2.47-2.43 (m, 2H), 2.23 (s, 3H), 1.79-1.68 (m, 2H), 1.62-1.55 (m, 2H), 1.47-1.38 (m, 2H)

### Step E. Procedure for Preparation of tert-butyl 6-(8-(benzo[d]thiazol-2-ylcarbamoyl)-3,4-dihydroisoquinolin-2(1H)-yl)-3-(2-methyl-3-((6-oxohexyl)oxy)phenyl)picolinate

To a solution of 6-(3-bromo-2-methylphenoxy)hexanal (446.94 mg, 1.57 mmol, 1.2 equiv.) in 1,4-dioxane (8 mL) was added KF (1.5 M, 2.61 mL, 3 equiv.), [2-(2-aminophenyl)phenyl]palladium(1+); bis(1-adamantyl)-butyl-phosphane; methanesulfonate (95.11 mg, 130.60 μmol, 0.1 equiv.), and tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (800 mg, 1.31 mmol, 1 equiv.). The mixture was stirred at 100° C. for 1 hour under N₂. Water (50 mL) was added, and the mixture was extracted with DCM 60 (30 mL×2), dried over anhydrous sodium sulfate, filtered, and concentrated under vacuum. The residue was purified by flash silica gel chromatography (Eluent of 0˜30% ethyl acetate/petroleum ether) to give tert-butyl 6-(8-(benzo[d]thiazol-2-ylcarbamoyl)-3,4-dihydroisoquinolin-2(1H)-yl)-3-(2-methyl-3-((6-oxohexyl)oxy)phenyl)picolinate (220 mg, 287.8 μmol, 22.0% yield, 90.3% purity) as a yellow solid.

MS (ESI) m/z: 691.4 [M+H]⁺

### Step F. Procedure for Preparation of tert-butyl 6-(8-(benzo[d]thiazol-2-ylcarbamoyl)-3,4-dihydroisoquinolin-2(1H)-yl)-3-(3-((6-(4-(3-(2,6-dioxopiperidin-3-yl)-1-methyl-1H-indazol-6-yl)piperazin-1-yl)hexyl)oxy)-2-methylphenyl)picolinate

To a solution of tert-butyl 6-(8-(benzo[d]thiazol-2-ylcarbamoyl)-3,4-dihydroisoquinolin-2(1H)-yl)-3-(2-methyl-3-((6-oxohexyl)oxy)phenyl)picolinate (90 mg, 130.27 μmol, 1 equiv.) in DCM (1 mL) was added NMM (13.18 mg, 130.27 μmol, 14.32 μL, 1 equiv.) and 3-(1-methyl-6-(piperazin-1-yl)-1H-indazol-3-yl)piperidine-2,6-dione (46.91 mg, 143.30 μmol, 1.1 equiv.). The mixture was stirred at 25° C. for 15 hours. Then NaBH₃CN (24.56 mg, 390.82 μmol, 3 equiv.) was added into the mixture and stirred at 25° C. for 1 hour. Water (50 mL) was added, and layers were separated. The organic layers were dried over anhydrous sodium sulfate, filtered, and concentrated under vacuum. The residue was purified by prep-TLC (SiO₂, DCM:MeOH=10:1) to give tert-butyl 6-(8-(benzo[d]thiazol-2-ylcarbamoyl)-3,4-dihydroisoquinolin-2(1H)-yl)-3-(3-((6-(4-(3-(2,6-dioxopiperidin-3-yl)-1-methyl-1H-indazol-6-yl)piperazin-1-yl)hexyl)oxy)-2-methylphenyl)picolinate (125 mg, 124.7 μmol, 95.7% yield) as a yellow oil.

MS (ESI) m/z: 1002.5 [M+H]⁺

### Step G. Procedure for Preparation of 6-(8-(benzo[d]thiazol-2-ylcarbamoyl)-3,4-dihydroisoquinolin-2(1H)-yl)-3-(3-((6-(4-(3-(2,6-dioxopiperidin-3-yl)-1-methyl-1H-indazol-6-yl)piperazin-1-yl)hexyl)oxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-((6-(4-(3-(2,6-dioxopiperidin-3-yl)-1-methyl-1H-indazol-6-yl)piperazin-1-yl)hexyl)oxy)-2-methylphenyl)picolinate (125 mg, 124.72 μmol, 1 equiv.) in DCM (2 mL) was added TFA (1.54 g, 13.47 mmol, 1 mL, 114.3 equiv.). The mixture was stirred at 40° C. for 12 hours. The reaction mixture was concentrated under reduced pressure to remove solvent. The residue was purified by prep-HPLC to give 6-(8-(benzo[d]thiazol-2-ylcarbamoyl)-3,4-dihydroisoquinolin-2(1H)-yl)-3-(3-((6-(4-(3-(2,6-dioxopiperidin-3-yl)-1-methyl-1H-indazol-6-yl)piperazin-1-yl)hexyl)oxy)-2-methylphenyl)picolinic acid (37.0 mg, 38.0 μmol, 30.5% yield, 97.1% purity) as a yellow solid.

MS (ESI) m/z: 947.1 [M+H]⁺

¹H NMR (400 MHz, DMSO-d₆) δ=13.02-12.73 (m, 1H), 10.85 (s, 1H), 9.58-9.35 (m, 1H), 8.02 (d, J=8.0 Hz, 1H), 7.78 (d, J=8.0 Hz, 1H), 7.62 (d, J=7.6 Hz, 1H), 7.56 (d, J=8.8 Hz, 1H), 7.49-7.42 (m, 3H), 7.40-7.32 (m, 2H), 7.10 (t, J=8.0 Hz, 1H), 6.99-6.94 (m, 3H), 6.89 (d, J=8.4 Hz, 1H), 6.63 (d, J=7.6 Hz, 1H), 4.98 (s, 2H), 4.30-4.25 (m, 1H), 3.98 (t, J=6.0 Hz, 2H), 3.91 (s, 3H), 3.60 (d, J=10.4 Hz, 6H), 3.18 (d, J=10.0 Hz, 4H), 3.07-2.99 (m, 4H), 2.65-2.58 (m, 2H), 2.35-2.28 (m, 1H), 2.19-2.15 (m, 1H), 1.90 (s, 3H), 1.80-1.69 (m, 4H), 1.55-1.46 (m, 2H), 1.44-1.34 (m, 2H)

### Example 166. Preparation of Compound 224b

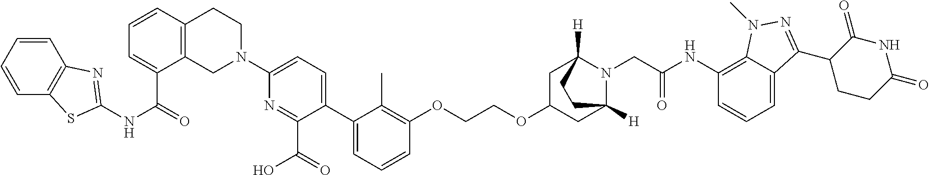 [SMILES: Cc1c(OCCOC2C[C@H]3CC[C@@H](C2)N3CC(=O)Nc2cccc3c(C4CCC(=O)NC4=O)nn(C)c23)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(2-(((1R,3r,5S)-8-(2-((3-(2,6-dioxopiperidin-3-yl)-1-methyl-1H-indazol-7-yl)amino)-2-oxoethyl)-8-azabicyclo[3.2.1]octan-3-yl)oxy)ethoxy)-2-methylphenyl)picolinic acid

### Step A. Procedure for Preparation of tert-butyl 6-[8-(1,3-benzothiazol-2-ylcarbamoyl)-3,4-dihydro-1H-isoquinolin-2-yl]-3-[3-[2-[[(1R,5S)-8-[2-[[3-(2,6-dioxo-3-piperidyl)-1-methyl-indazol-7-yl]amino]-2-oxo-ethyl]-8-azabicyclo[3.2.1]octan-3-yl]oxy]ethoxy]-2-methyl-phenyl]pyridine-2-carboxylate

To a solution of 2-[(1R,5S)-3-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]ethoxy]-8-azabicyclo[3.2.1]octan-8-yl]acetic acid (170 mg, 211.45 μmol, 1.0 equiv.) and 3-(7-amino-1-methyl-indazol-3-yl)piperidine-2,6-dione (60.07 mg, 232.60 μmol, 1.1 equiv.) in DMF (2 mL) was added HATU (104.52 mg, 274.89 μmol, 1.3 equiv.) and DIPEA (81.98 mg, 634.36 μmol, 110.49 μL, 3.0 equiv.). The mixture was stirred at 25° C. for 16 hours. The reaction mixture was added to H₂O (5.0 ml). The mixture was triturated with H₂O (5.0 mL) at 25° C. for 30 min and filtered to give tert-butyl 6-[8-(1,3-benzothiazol-2-ylcarbamoyl)-3,4-dihydro-1H-isoquinolin-2-yl]-3-[3-[2-[[(1R,5S)-8-[2-[[3-(2,6-dioxo-3-piperidyl)-1-methyl-indazol-7-yl]amino]-2-oxo-ethyl]-8-azabicyclo[3.2.1]octan-3-yl]oxy]ethoxy]-2-methyl-phenyl]pyridine-2-carboxylate (200 mg, 191.5 μmol, 90.5% yield) as a brown solid.

MS (ESI) m/z: 1044.6 [M+H]⁺.

### Step B. Procedure for Preparation of 6-(8-(benzo[d]thiazol-2-ylcarbamoyl)-3,4-dihydroisoquinolin-2(1H)-yl)-3-(3-(2-(((1R,3r,5S)-8-(2-((3-(2,6-dioxopiperidin-3-yl)-1-methyl-1H-indazol-7-yl)amino)-2-oxoethyl)-8-azabicyclo[3.2.1]octan-3-yl)oxy)ethoxy)-2-methylphenyl)picolinic acid

A solution of tert-butyl 6-[8-(1,3-benzothiazol-2-ylcarbamoyl)-3,4-dihydro-1H-isoquinolin-2-yl]-3-[3-[2-[[(1R,5S)-8-[2-[[3-(2,6-dioxo-3-piperidyl)-1-methyl-indazol-7-yl]amino]-2-oxo-ethyl]-8-azabicyclo[3.2.1]octan-3-yl]oxy]ethoxy]-2-methyl-phenyl]pyridine-2-carboxylate (200 mg, 191.53 μmol, 1.0 equiv.) in DCM (1 mL) and TFA (1 mL) was stirred at 25° C. for 16 hours. The reaction mixture was concentrated under reduced pressure to remove DCM. The resulting residue was purified by prep-HPLC to give 6-(8-(benzo[d]thiazol-2-ylcarbamoyl)-3,4-dihydroisoquinolin-2(1H)-yl)-3-(3-(2-(((1R,3r,5S)-8-(2-((3-(2,6-dioxopiperidin-3-yl)-1-methyl-1H-indazol-7-yl)amino)-2-oxoethyl)-8-azabicyclo[3.2.1]octan-3-yl)oxy)ethoxy)-2-methylphenyl)picolinic acid (31.4 mg, 29.1 μmol, 15.1% yield, 91.3% purity) as a yellow solid.

MS (ESI) m/z: 988.4 [M+H]⁺.

¹H NMR (400 MHz, DMSO-d₆) δ=13.01-12.74 (m, 1H), 12.69-12.21 (m, 1H), 10.90 (s, 1H), 10.06 (d, J=8.4 Hz, 1H), 8.13 (s, 1H), 8.03 (d, J=8.0 Hz, 1H), 7.79 (d, J=8.4 Hz, 1H), 7.60 (dd, J=7.6, 14.0 Hz, 2H), 7.49-7.43 (m, 3H), 7.40-7.29 (m, 3H), 7.13-7.06 (m, 2H), 6.97 (d, J=8.8 Hz, 1H), 6.92 (d, J=8.4 Hz, 1H), 6.65 (d, J=8.0 Hz, 1H), 4.98 (s, 2H), 4.38 (dd, J=4.8, 10.4 Hz, 1H), 4.13-4.07 (m, 5H), 3.92 (t, J=6.0 Hz, 2H), 3.71 (s, 2H), 3.64-3.60 (m, 1H), 3.44-3.40 (m, 2H), 3.03 (t, J=5.6 Hz, 2H), 2.65-2.55 (m, 5H), 2.37 (s, 1H), 2.20-2.04 (m, 6H), 1.92 (s, 3H), 1.89-1.82 (m, 2H).

### Example 167. Preparation of Compound 225

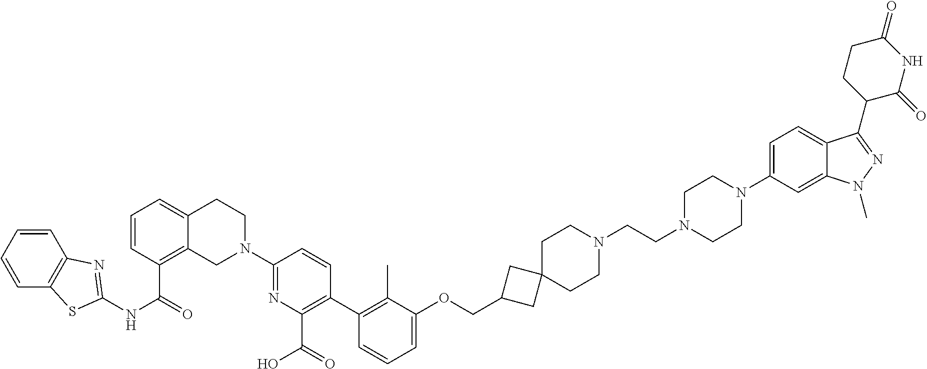 [SMILES: Cc1c(OCC2CC3(CCN(CCN4CCN(c5ccc6c(C7CCC(=O)NC7=O)nn(C)c6c5)CC4)CC3)C2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-((7-(2-(4-(3-(2,6-dioxopiperidin-3-yl)-1-methyl-1H-indazol-6-yl)piperazin-1-yl)ethyl)-7-azaspiro[3.5]nonan-2-yl)methoxy)-2-methylphenyl)picolinic acid

### Step A. Procedure for Preparation of 6-(8-(benzo[d]thiazol-2-ylcarbamoyl)-3,4-dihydroisoquinolin-2(1H)-yl)-3-(3-((7-(2-(4-(3-(2,6-dioxopiperidin-3-yl)-1-methyl-1H-indazol-6-yl)piperazin-1-yl)ethyl)-7-azaspiro[3.5]nonan-2-yl)methoxy)-2-methylphenyl)picolinic acid

A mixture of 3-(1-methyl-6-piperazin-1-yl-indazol-3-yl)piperidine-2,6-dione (27.44 mg, 83.82 μmol, 1.2 equiv.), NaBH(OAc)₃ (44.41 mg, 209.54 μmol, 3.0 equiv.), N N (7.06 mg, 69.85 μmol, 7.68 μL, 1.0 equiv.) in DCM (0.5 mL) and isopropanol (0.5 mL) was stirred at 0° C. for 5 min. 6-[8-(1,3-Benzothiazol-2-ylcarbamoyl)-3,4-dihydro-1H-isoquinolin-2-yl]-3-[2-methyl-3-[[7-(2-oxoethyl)-7-azaspiro[3.5]nonan-2-yl]methoxy]phenyl]pyridine-2-carboxylic acid (50 mg, 69.85 μmol, 1.0 equiv.) was then added to the mixture, which was stirred at 0° C. for 30 min. The mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-((7-(2-(4-(3-(2,6-dioxopiperidin-3-yl)-1-methyl-1H-indazol-6-yl)piperazin-1-yl)ethyl)-7-azaspiro[3.5]nonan-2-yl)methoxy)-2-methylphenyl)picolinic acid (21.1 mg, 19.9 μmol, 9.5% yield, 96.8% purity) as a white solid

MS (ESI) m/z: 1027.4 [M+H]⁺

¹H NMR (400 MHz, DMSO-d₆) δ=10.84 (s, 1H), 8.02 (d, J=8.0 Hz, 1H), 7.78 (d, J=8.0 Hz, 1H), 7.62 (d, J=7.6 Hz, 1H), 7.50-7.33 (m, 6H), 7.07 (t, J=8.0 Hz, 1H), 6.93-6.81 (m, 4H), 6.65 (d, J=7.6 Hz, 1H), 4.97 (s, 2H), 4.25 (dd, J=5.2, 9.2 Hz, 1H), 3.92-3.87 (m, 7H), 3.21-3.17 (m, 6H), 3.02-3.0 (m, 4H), 2.65-2.54 (m, 8H), 2.38-2.24 (m, 4H), 2.19-2.12 (m, 1H), 1.92-1.84 (m, 5H), 1.67-1.57 (m, 4H), 1.50-1.45 (m, 2H)

### Example 168. Preparation of Compound 227a

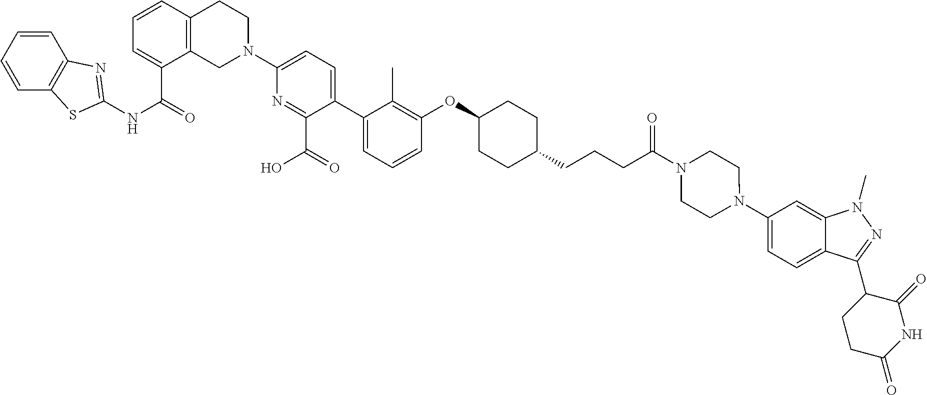 [SMILES: Cc1c(O[C@H]2CC[C@H](CCCC(=O)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)-4-oxobutyl)cyclohexyl)oxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)-4-oxobutyl)cyclohexyl)oxy)-2-methylphenyl)picolinate

To a solution of 4-((1r,4s)-4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)cyclohexyl)butanoic acid (100 mg, 131.42 μmol, 1 equiv.) in pyridine (1 mL) was added EDCI (32.75 mg, 170.84 μmol, 1.3 equiv.) and 3-(1-methyl-6-piperazin-1-yl-indazol-3-yl)piperidine-2,6-dione (51.63 mg, 157.70 μmol, 1.2 equiv.). The mixture was stirred at 60° C. for 12 hours. The reaction was diluted with water (20 mL) and extracted with DCM (20 mL×2). The combined organic layers were washed with brine (30 mL×1), dried over Na₂SO₄, filtered, and concentrated under reduced pressure to give a residue. The residue was purified by prep-TLC (SiO₂), to give tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)-4-oxobutyl)cyclohexyl)oxy)-2-methylphenyl)picolinate (100 mg, 93.4 μmol, 71.1% yield) as a yellow solid

MS (ESI) m/z: 1170.8 [M+H]⁺.

### Step B. Procedure for Preparation of 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)-4-oxobutyl)cyclohexyl)oxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)-4-oxobutyl)cyclohexyl)oxy)-2-methylphenyl)picolinate (100 mg, 93.43 μmol, 1 equiv.) in DCM (1 mL) was added TFA (1.54 g, 13.51 mmol, 1 mL, 144.56 equiv.). The mixture was stirred at 40° C. for 1 hour. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)-4-oxobutyl)cyclohexyl)oxy)-2-methylphenyl)picolinic acid (28.6 mg, 27.2 μmol, 29.1% yield, 96.4% purity) as a yellow solid.

MS (ESI) m/z: 1015.0 [M+H]⁺.

¹H NMR (400 MHz, DMSO-d₆) δ=12.87 (s, 1H), 12.69-12.43 (m, 1H), 10.86 (s, 1H), 8.04 (d, J=8.0 Hz, 1H), 7.80 (d, J=7.6 Hz, 1H), 7.63 (d, J=6.4 Hz, 1H), 7.53 (d, J=8.8 Hz, 1H), 7.50-7.43 (m, 3H), 7.40-7.33 (m, 2H), 7.11-7.05 (m, 1H), 6.98-6.88 (m, 4H), 6.61 (d, J=7.6 Hz, 1H), 4.98 (s, 2H), 4.30-4.25 (m, 1H), 4.23-4.17 (m, 1H), 3.92 (s, 2H), 3.90 (s, 3H), 3.63 (s, 4H), 3.24 (s, 2H), 3.18 (d, J=1.2 Hz, 2H), 3.05-3.01 (m, 2H), 2.63-2.59 (m, 2H), 2.38-2.33 (m, 2H), 2.19-2.13 (m, 1H), 2.11-2.05 (m, 2H), 1.87 (s, 3H), 1.82-1.75 (m, 2H), 1.59-1.52 (m, 2H), 1.40-1.21 (m, 6H), 1.11-1.01 (m, 2H)

### Example 169. Preparation of Compound 230

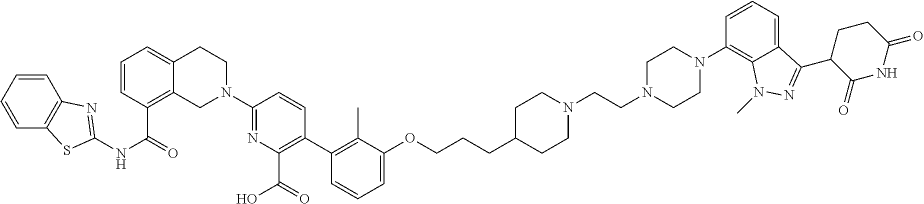 [SMILES: Cc1c(OCCCC2CCN(CCN3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-(1-(2-(4-(3-(2,6-dioxopiperidin-3-yl)-1-methyl-1H-indazol-7-yl)piperazin-1-yl)ethyl)piperidin-4-yl)propoxy)-2-methylphenyl)picolinic acid

### Step A. Procedure for Preparation of tert-butyl 4-[3-(3-bromo-2-methyl-phenoxy)propyl]piperidine-1-carboxylate

To a solution of 3-bromo-2-methyl-phenol (1 g, 5.35 mmol, 1 equiv.) and tert-butyl 4-(3-bromopropyl)piperidine-1-carboxylate (1.64 g, 5.35 mmol, 1 equiv.) in CH₃CN (4 mL) was added K₂CO₃ (2.22 g, 16.04 mmol, 3 equiv.). The mixture was stirred at 60° C. for 2 hours. The reaction mixture was concentrated under reduced pressure to give a residue. The residue was purified by column chromatography (SiO₂, petroleum ether/ethyl acetate=I/O to 10/1) to give tert-butyl 4-[3-(3-bromo-2-methyl-phenoxy)propyl]piperidine-1-carboxylate (2.2 g, 5.3 mmol, 99.7% yield) as a colorless oil.

MS (ESI) m/z: 312.4 [M+H]⁺

¹H NMR (400 MHz, CDCl₃) δ=7.14 (d, J=8.0 Hz, 1H), 6.99 (t, J=8.0 Hz, 1H), 6.76 (d, J=8.4 Hz, 1H), 4.14-4.06 (m, 2H), 3.94 (t, J=6.4 Hz, 2H), 2.69 (t, J=2.4, 12.8 Hz, 2H), 2.32 (s, 3H), 1.88-1.79 (m, 2H), 1.70 (d, J=12.8 Hz, 2H), 1.48-1.43 (m, 12H), 1.18-1.08 (m, 2H)

### Step B. Procedure for Preparation of 4-[3-(3-bromo-2-methyl-phenoxy)propyl]piperidine

To a solution of tert-butyl 4-[3-(3-bromo-2-methyl-phenoxy)propyl]piperidine-1-carboxylate (2.2 g, 5.34 mmol, 1 equiv.) in HCl/EtOAc (10 mL). The mixture was stirred at 25° C. for 1 hour. The reaction mixture was filtered and concentrated under reduced pressure to give a residue to give 4-[3-(3-bromo-2-methyl-phenoxy)propyl]piperidine (1.6 g, 5.1 mmol, 96.0% yield) as a white solid.

MS (ESI) m/z: 314.4 [M+H]⁺

¹HNMR (400 MHz, CDCl₃) δ=7.14 (d, J=8.0 Hz, 1H), 6.99 (t, J=8.0 Hz, 1H), 6.76 (d, J=8.4 Hz, 1H), 4.14-4.06 (m, 2H), 3.94 (t, J=6.4 Hz, 2H), 2.69 (t, J=2.4, 12.8 Hz, 2H), 2.32 (s, 3H), 1.88-1.79 (m, 2H), 1.70 (d, J=12.8 Hz, 2H), 1.48-1.43 (m, 12H), 1.18-1.08 (m, 2H)

### Step C. Procedure for Preparation of 4-[3-(3-bromo-2-methyl-phenoxy)propyl]-1-(2,2-diethoxyethyl)piperidine

To a solution of 4-[3-(3-bromo-2-methyl-phenoxy)propyl]piperidine (1.5 g, 4.80 mmol, 1 equiv.) 2-bromo-1,1-diethoxy-ethane (946.71 mg, 4.80 mmol, 722.68 μL, 1 equiv.) in CH₃CN (5 mL) was added K₂CO₃ (1.99 g, 14.41 mmol, 3 equiv.) and KI (797.46 mg, 4.80 mmol, 1 equiv.). The mixture was stirred at 80° C. for 8 hours. The reaction mixture was concentrated under reduced pressure to give a residue. The residue was purified by column chromatography (SiO₂, ethyl acetate/MeOH=1/0 to 20/1) to give 4-[3-(3-bromo-2-methyl-phenoxy)propyl]-1-(2,2-diethoxyethyl)piperidine (1.7 g, 3.9 mmol, 82.6% yield) as a yellow oil.

MS (ESI) m/z: 430.5 [M+H]⁺

### Step D. Procedure for Preparation of tert-butyl 6-(8-(benzo[d]thiazol-2-ylcarbamoyl)-3,4-dihydroisoquinolin-2(1H)-yl)-3-(3-(3-(1-(2,2-diethoxyethyl)piperidin-4-yl)propoxy)-2-methylphenyl)picolinate

A mixture of 4-[3-(3-bromo-2-methyl-phenoxy)propyl]-1-(2,2-diethoxyethyl)piperidine (800 mg, 1.87 mmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (1.14 g, 1.87 mmol, 1 equiv.), [2-(2-aminophenyl)phenyl]palladium(1+); bis(1-adamantyl)-butyl-phosphane; methanesulfonate (136.00 mg, 186.74 μmol, 0.1 equiv.), and KF (1.5 M, 3.73 mL, 3 equiv.) in dioxane (5 mL) and H₂O (1 mL) was degassed and purged with N₂ three times, and then the mixture was stirred at 100° C. for 1 hour under microwave. The reaction mixture was concentrated under reduced pressure to give a residue. The residue was purified by column chromatography (SiO₂, ethyl acetate/MeOH=1/0 to 10/1) to give tert-butyl 6-(8-(benzo[d]thiazol-2-ylcarbamoyl)-3,4-dihydroisoquinolin-2(1H)-yl)-3-(3-(3-(1-(2,2-diethoxyethyl)piperidin-4-yl)propoxy)-2-methylphenyl)picolinate (1.5 g, 1.7 mmol, 91.9% yield, 95.5% purity) as a yellow oil.

MS (ESI) m/z: 834.8 [M+H]⁺

¹H NMR (400 MHz, CDCl₃) δ=7.85 (d, J=8.0 Hz, 1H), 7.59 (dd, J=7.2, 15.6 Hz, 2H), 7.40-7.30 (m, 5H), 7.14-7.06 (m, 1H), 6.90 (d, J=8.8 Hz, 1H), 6.80 (d, J=8.4 Hz, 1H), 6.69 (d, J=7.6 Hz, 1H), 5.12-4.95 (m, 2H), 4.81-4.60 (m, 1H), 4.12-4.08 (m, 2H), 3.97 (t, J=6.4 Hz, 2H), 3.72-3.68 (m, 2H), 3.61-3.54 (m, 2H), 3.07 (t, J=5.2 Hz, 4H), 2.64-2.52 (m, 2H), 2.05 (s, 3H), 1.84-1.80 (m, 2H), 1.69-1.55 (m, 9H), 1.24-1.20 (m, 6H), 1.15 (s, 9H).

### Step E. Procedure for Preparation of 6-(8-(benzo[d]thiazol-2-ylcarbamoyl)-3,4-dihydroisoquinolin-2(1H)-yl)-3-(2-methyl-3-(3-(1-(2-oxoethyl)piperidin-4-yl)propoxy)phenyl)picolinic acid

To a solution of tert-butyl 6-(8-(benzo[d]thiazol-2-ylcarbamoyl)-3,4-dihydroisoquinolin-2(1H)-yl)-3-(3-(3-(1-(2,2-diethoxyethyl)piperidin-4-yl)propoxy)-2-methylphenyl)picolinate (200 mg, 239.79 μmol, 1.0 equiv.) was added HCOOH (11.52 mg, 239.79 μmol, 2 mL, 1.0 equiv.). The mixture was stirred at 90° C. for 1.5 hours. The reaction mixture was filtered and concentrated under reduced pressure to give 6-(8-(benzo[d]thiazol-2-ylcarbamoyl)-3,4-dihydroisoquinolin-2(1H)-yl)-3-(2-methyl-3-(3-(1-(2-oxoethyl)piperidin-4-yl)propoxy)phenyl)picolinic acid (100 mg, crude) as a black oil, which was used in the next step without further purification.

MS (ESI) m/z: 704.3 [M+H]⁺.

### Step F. Procedure for Preparation of 6-(8-(benzo[d]thiazol-2-ylcarbamoyl)-3,4-dihydroisoquinolin-2(1H)-yl)-3-(3-(3-(1-(2-(4-(3-(2,6-dioxopiperidin-3-yl)-1-methyl-11H-indazol-7-yl)piperazin-1-yl)ethyl)piperidin-4-yl)propoxy)-2-methylphenyl)picolinic acid

To a solution of 3-(1-methyl-7-(piperazin-1-yl)-1H-indazol-3-yl)piperidine-2,6-dione (46.51 mg, 142.08 μmol, 1.0 equiv.) and NaBH(OAc)₃ (90.34 mg, 426.23 μmol, 3.0 equiv.) in DCM (1 mL) and IPA (1 mL) was added 6-(8-(benzo[d]thiazol-2-ylcarbamoyl)-3,4-dihydroisoquinolin-2(1H)-yl)-3-(2-methyl-3-(3-(1-(2-oxoethyl)piperidin-4-yl)propoxy)phenyl)picolinic acid (100 mg, 142.08 μmol, 1.0 equiv.) at 0° C. The mixture was stirred at 0° C. for 1 hour. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(3-(1-(2-(4-(3-(2,6-dioxopiperidin-3-yl)-1-methyl-1H-indazol-7-yl)piperazin-1-yl)ethyl)piperidin-4-yl)propoxy)-2-methylphenyl)picolinic acid (19.3 mg, 16.8 μmol, 11.8% yield, 88.2% purity) as a yellow solid.

MS (ESI) m/z: 1015.4 [M+H]⁺.

¹H NMR (400 MHz, DMSO-d₆) δ=10.87 (s, 1H), 8.00 (d, J=7.6 Hz, 1H), 7.76 (d, J=8.0 Hz, 1H), 7.62 (d, J=7.2 Hz, 1H), 7.46-7.29 (m, 6H), 7.10-6.99 (m, 3H), 6.85 (d, J=8.0 Hz, 2H), 6.65 (d, J=7.6 Hz, 1H), 4.96 (s, 2H), 4.34-4.31 (m, 1H), 4.20 (s, 3H), 3.99-3.93 (m, 2H), 3.88-3.87 (m, 2H), 2.99-2.91 (m, 6H), 2.69-2.59 (m, 4H), 2.53 (s, 4H), 2.34-2.28 (m, 2H), 2.21-2.10 (m, 2H), 2.10-1.95 (m, 3H), 1.90 (s, 3H), 1.78-1.66 (m, 3H), 1.65-1.54 (m, 2H), 1.37-1.21 (m, 5H).

### Example 170. Preparation of Compound 232

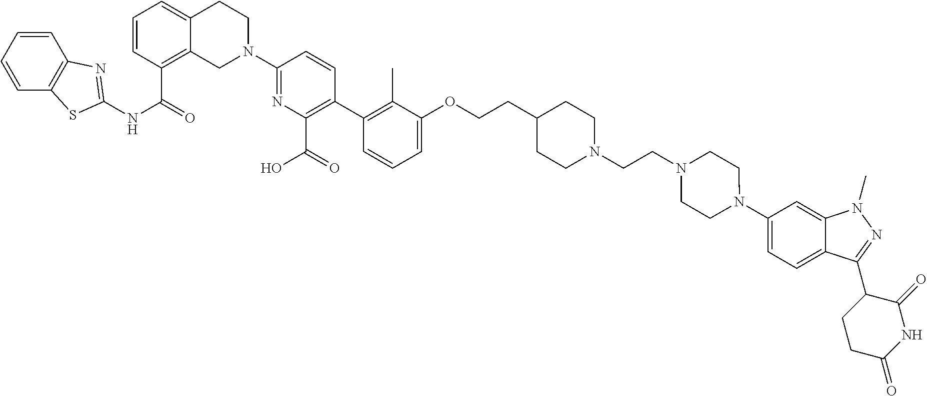 [SMILES: Cc1c(OCCC2CCN(CCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[2-[1-[2-[4-[3-(2,6-dioxo-3-piperidyl)-1-methyl-indazol-6-yl]piperazin-1-yl]ethyl]-4-piperidyl]ethoxy]-2-methyl-phenyl]pyridine-2-carboxylic acid

### Step A. Procedure for Preparation of 6-[8-(1,3-benzothiazol-2-ylcarbamoyl)-3,4-dihydro-1H-isoquinolin-2-yl]-3-[3-[2-[1-[2-[4-[3-(2,6-dioxo-3-piperidyl)-1-methyl-indazol-6-yl]piperazin-1-yl]ethyl]-4-piperidyl]ethoxy]-2-methyl-phenyl]pyridine-2-carboxylic acid

To a solution of 3-(1-methyl-6-piperazin-1-yl-indazol-3-yl)piperidine-2,6-dione (72.61 mg, 221.80 μmol, 0.9 equiv.) and NaBH(OAc)₃ (156.69 mg, 739.32 μmol, 3.0 equiv.) in DCM (1.5 mL) and IPA (1.5 mL) was added 6-[8-(1,3-benzothiazol-2-ylcarbamoyl)-3,4-dihydro-1H-isoquinolin-2-yl]-3-[2-methyl-3-[2-[1-(2-oxoethyl)-4-piperidyl]ethoxy]phenyl]pyridine-2-carboxylic acid (170 mg, 246.44 μmol, 1.0 equiv.) at 0° C. slowly. The mixture was stirred at 0° C. for 1 hour. Then the mixture was warmed to 25° C. and stirred at 25° C. for 12 hours. The mixture was concentrated under reduced pressure to remove DCM and IPA and diluted with DMF (3 mL). The resulting mixture was purified by prep-HPLC to give 6-[8-(1,3-benzothiazol-2-ylcarbamoyl)-3,4-dihydro-1H-isoquinolin-2-yl]-3-[3-[2-[1-[2-[4-[3-(2,6-dioxo-3-piperidyl)-1-methyl-indazol-6-yl]piperazin-1-yl]ethyl]-4-piperidyl]ethoxy]-2-methyl-phenyl]pyridine-2-carboxylic acid (19.3 mg, 17.4 μmol, 7.0% yield, 94.2% purity) as a yellow solid.

MS (ESI) m/z: 1001.5 [M+H]⁺

¹H NMR (400 MHz, DMSO-d₆) δ=10.84 (s, 1H), 8.02 (d, J=8.0 Hz, 1H), 7.78 (d, J=8.0 Hz, 1H), 7.62 (d, J=7.6 Hz, 1H), 7.52-7.29 (m, 6H), 7.11-7.04 (m, 1H), 6.95-6.85 (m, 3H), 6.82 (s, 1H), 6.64 (d, J=7.6 Hz, 1H), 4.97 (s, 2H), 4.28-4.22 (m, 1H), 3.98 (d, J=5.2 Hz, 2H), 3.93-3.86 (m, 5H), 3.22-2.17 (m, 4H), 3.01 (t, J=5.6 Hz, 2H), 2.96 (d, J=10.0 Hz, 2H), 2.65-2.59 (m, 2H), 2.59-2.52 (m, 7H), 2.35-2.24 (m, 2H), 2.18-2.12 (m, 1H), 2.12-2.04 (m, 2H), 1.90 (s, 3H), 1.72-1.63 (m, 4H), 1.55-1.47 (m, 1H), 1.27-1.17 (m, 2H)

### Example 171. Preparation of Compound 233

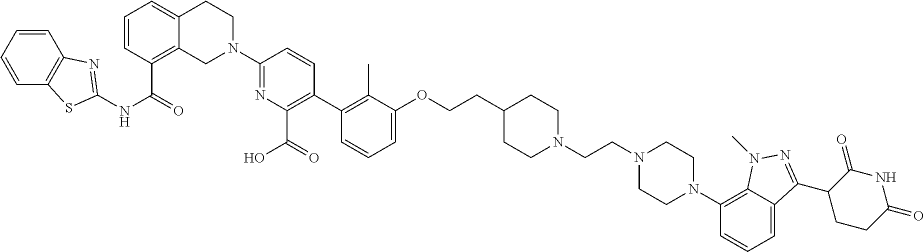 [SMILES: Cc1c(OCCC2CCN(CCN3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[2-[1-[2-[4-[3-(2,6-dioxo-3-piperidyl)-1-methyl-indazol-7-yl]piperazin-1-yl]ethyl]-4-piperidyl]ethoxy]-2-methyl-phenyl]pyridine-2-carboxylic acid

### Step A. Procedure for Preparation of 6-[8-(1,3-benzothiazol-2-ylcarbamoyl)-3,4-dihydro-1H-isoquinolin-2-yl]-3-[3-[2-[1-[2-[4-[3-(2,6-dioxo-3-piperidyl)-1-methyl-indazol-7-yl]piperazin-1-yl]ethyl]-4-piperidyl]ethoxy]-2-methyl-phenyl]pyridine-2-carboxylic acid

To a solution of 3-(1-methyl-7-piperazin-1-yl-indazol-3-yl)piperidine-2,6-dione (46.98 mg, 143.52 μmol, 1.1 equiv.) and NaBH(OAc)₃ (82.95 mg, 391.41 μmol, 3 equiv.) in DCM (1.5 mL) and IPA (1.5 mL) was added 6-[8-(1,3-benzothiazol-2-ylcarbamoyl)-3,4-dihydro-1H-isoquinolin-2-yl]-3-[2-methyl-3-[2-[1-(2-oxoethyl)-4-piperidyl]ethoxy]phenyl]pyridine-2-carboxylic acid (90 mg, 130.47 μmol, 1 equiv.) at 0° C. slowly. The mixture was stirred at 0° C. for 1 hour. Then the mixture was warmed to 25° C. and stirred at 25° C. for 12 hours. The mixture was concentrated under reduced pressure to remove DCM and IPA and diluted with DMF (2 mL). The resulting mixture was purified by prep-HPLC to give 6-[8-(1,3-benzothiazol-2-ylcarbamoyl)-3,4-dihydro-1H-isoquinolin-2-yl]-3-[3-[2-[1-[2-[4-[3-(2,6-dioxo-3-piperidyl)-1-methyl-indazol-7-yl]piperazin-1-yl]ethyl]-4-piperidyl]ethoxy]-2-methyl-phenyl]pyridine-2-carboxylic acid (12.5 mg, 11.8 μmol, 9.0% yield, 98.3% purity) as a yellow solid.

MS (ESI) m/z: 1001.7 [M+H]⁺

¹H NMR (400 MHz, DMSO-d₆) δ=10.89 (s, 1H), 8.02 (d, J=8.0 Hz, 1H), 7.78 (d, J=8.0 Hz, 1H), 7.62 (d, J=7.2 Hz, 1H), 7.48-7.40 (m, 3H), 7.40-7.32 (m, 3H), 7.11-7.06 (m, 1H), 7.04-6.99 (m, 2H), 6.96-6.86 (m, 2H), 6.64 (d, J=7.6 Hz, 1H), 4.97 (s, 2H), 4.36-4.31 (m, 1H), 4.23 (s, 3H), 3.99 (d, J=5.2 Hz, 2H), 3.91 (t, J=6.0 Hz, 2H), 3.15-3.07 (m, 5H), 3.03-3.00 (m, 2H), 2.76-2.57 (m, 10H), 2.38-2.28 (m, 4H), 2.20-2.13 (m, 1H), 1.90 (s, 3H), 1.78-1.66 (m, 4H), 1.63-1.55 (m, 1H), 1.36-1.25 (m, 2H)

### Example 172. Preparation of Compound 234

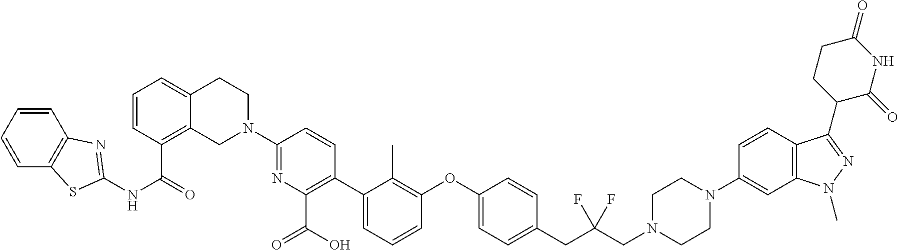 [SMILES: Cc1c(Oc2ccc(CC(F)(F)CN3CCN(c4ccc5c(C6CCC(=O)NC6=O)nn(C)c5c4)CC3)cc2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-6-yl]piperazin-1-yl]-2,2-difluoro-propyl]phenoxy]-2-methyl-phenyl]pyridine-2-carboxylic acid

### Step A. Procedure for Preparation of 3-(6-(4-(3-(4-(3-bromo-2-methylphenoxy)phenyl)-2,2-difluoropropyl)piperazin-1-yl)-1-methyl-1H-indazol-3-yl)piperidine-2,6-dione

A mixture of 3-(4-(3-bromo-2-methylphenoxy)phenyl)-2,2-difluoropropyl trifluoromethanesulfonate (120 mg, 245.27 μmol, 1 equiv.), 3-(1-methyl-6-piperazin-1-yl-indazol-3-yl)piperidine-2,6-dione (64.24 mg, 196.22 μmol, 0.8 equiv.) and K₂CO₃ (84.75 mg, 613.18 μmol, 2.5 equiv.) in CH₃CN (1.5 mL) was degassed and purged with N₂ and stirred at 50° C. for 10 hours under N₂ atmosphere. The reaction mixture was concentrated under reduced pressure to give a residue. The residue was purified by flash silica gel chromatography (Eluent of 0˜50% ethyl acetate:petroleum ether) to give 3-(6-(4-(3-(4-(3-bromo-2-methylphenoxy)phenyl)-2,2-difluoropropyl)piperazin-1-yl)-1-methyl-1H-indazol-3-yl)piperidine-2,6-dione (80 mg, 120.0 μmol, 48.9% yield) as a colorless oil.

MS (ESI) m/z: 668.2 [M+H]⁺.

### Step B. Procedure for Preparation of tert-butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-6-yl]piperazin-1-yl]-2,2-difluoro-propyl]phenoxy]-2-methyl-phenyl]pyridine-2-carboxylate

A mixture of 3-(6-(4-(3-(4-(3-bromo-2-methylphenoxy)phenyl)-2,2-difluoropropyl)piperazin-1-yl)-1-methyl-1H-indazol-3-yl)piperidine-2,6-dione (80 mg, 120.02 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (102.92 mg, 168.03 μmol, 1.4 equiv.), [2-(2-aminophenyl)phenyl]palladium(1⁺); bis(1-adamantyl)-butyl-phosphane; methanesulfonate (17.48 mg, 24.00 μmol, 0.2 equiv.), and KF (1.5 M, 120.02 μL, 1.5 equiv.) in dioxane (0.1 mL) was degassed and purged with N₂ and stirred at 100° C. for 1 hour under microwave. The reaction mixture was filtered and concentrated under reduced pressure to give a residue. The residue was purified by flash silica gel chromatography (Eluent of 0˜50% ethyl acetate/petroleum ether) to give tert-butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-6-yl]piperazin-1-yl]-2,2-difluoro-propyl]phenoxy]-2-methyl-phenyl]pyridine-2-carboxylate (110 mg, 102.5 μmol, 85.4% yield) as a colorless oil.

MS (ESI) m/z: 1072.8 [M+H]⁺.

### Step C. Procedure for Preparation of 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-6-yl]piperazin-1-yl]-2,2-difluoro-propyl]phenoxy]-2-methyl-phenyl]pyridine-2-carboxylic acid

tert-Butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-6-yl]piperazin-1-yl]-2,2-difluoro-propyl]phenoxy]-2-methyl-phenyl]pyridine-2-carboxylate (110 mg, 102.59 μmol, 1 equiv.) in TFA (0.5 mL) and DCM (0.5 mL) was stirred at 25° C. for 10 hours. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-6-yl]piperazin-1-yl]-2,2-difluoro-propyl]phenoxy]-2-methyl-phenyl]pyridine-2-carboxylic acid (39.5 mg, 37.8 μmol, 36.9% yield, 97.4% purity) as an off-white solid

MS (ESI) m/z: 1016.5 [M+H]⁺.

¹H NMR (400 MHz, DMSO-d₆) δ=12.98-12.47 (m, 2H), 10.85 (s, 1H), 8.03 (d, J=8.0 Hz, 1H), 7.79 (d, J=8.0 Hz, 1H), 7.63 (d, J=7.6 Hz, 1H), 7.55-7.43 (m, 4H), 7.36 (td, J=7.6, 11.6 Hz, 2H), 7.27 (d, J=8.4 Hz, 2H), 7.22-7.16 (m, 1H), 7.01 (d, J=8.8 Hz, 1H), 6.95-6.82 (m, 6H), 5.00 (s, 2H), 4.26 (dd, J=5.2, 9.2 Hz, 1H), 3.96-3.90 (m, 2H), 3.89 (s, 3H), 3.30-3.19 (m, 6H), 3.10-3.00 (m, 2H), 2.77-2.54 (m, 8H), 2.32-2.13 (m, 2H), 1.89 (s, 3H)

F NMR (400 MHz, DMSO-d₆) δ=−96.27

### Example 173. Preparation of Compound 238

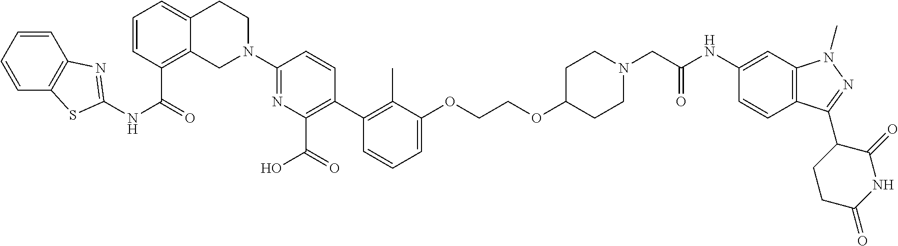 [SMILES: Cc1c(OCCOC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(2-((1-(2-((3-(2,6-dioxopiperidin-3-yl)-1-methyl-1H-indazol-6-yl)amino)-2-oxoethyl)piperidin-4-yl)oxy)ethoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(2-((1-(2-((3-(2,6-dioxopiperidin-3-yl)-1-methyl-1H-indazol-6-yl)amino)-2-oxoethyl)piperidin-4-yl)oxy)ethoxy)-2-methylphenyl)picolinate

A mixture of 2-(4-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethoxy)piperidin-1-yl)acetic acid (110 mg, 141.40 μmol, 1 equiv.) and 3-(6-amino-1-methyl-1H-indazol-3-yl)piperidine-2,6-dione (43.82 mg, 169.68 μmol, 1.2 equiv.) in pyridine (1.1 mL) was added EDCI (40.66 mg, 212.10 μmol, 1.5 equiv.), and then the mixture was stirred at 25° C. for 20 hours. The reaction mixture was partitioned between H₂O (3 mL) and ethyl acetate (8 mL). The organic phase was separated, washed with sat. NaCl aq. (2 mL×3), dried over (Na₂SO₄), filtered, and concentrated under reduced pressure to give tert-butyl 6-(8-(benzo[d]thiazol-2-ylcarbamoyl)-3,4-dihydroisoquinolin-2(1H)-yl)-3-(3-(2-((1-(2-((3-(2,6-dioxopiperidin-3-yl)-1-methyl-1H-indazol-6-yl)amino)-2-oxoethyl)piperidin-4-yl)oxy)ethoxy)-2-methylphenyl)picolinate (163 mg, crude) as a red solid.

MS (ESI) m/z: 1018.7 [M+H]⁺.

### Step B. Procedure for Preparation of 6-(8-(benzo[d]thiazol-2-ylcarbamoyl)-3,4-dihydroisoquinolin-2(1H)-yl)-3-(3-(2-((1-(2-((3-(2,6-dioxopiperidin-3-yl)-1-methyl-11H-indazol-6-yl)amino)-2-oxoethyl)piperidin-4-yl)oxy)ethoxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(3-(2-((1-(2-((3-(2,6-dioxopiperidin-3-yl)-1-methyl-1H-indazol-6-yl)amino)-2-oxoethyl)piperidin-4-yl)oxy)ethoxy)-2-methylphenyl)picolinate (150 mg, 147.32 μmol, 1 equiv.) in TFA (0.5 mL) and DCM (1.5 mL) was stirred at 25° C. for 18 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(2-((1-(2-((3-(2,6-dioxopiperidin-3-yl)-1-methyl-1H-indazol-6-yl)amino)-2-oxoethyl)piperidin-4-yl)oxy)ethoxy)-2-methylphenyl)picolinic acid (54.21 mg, 55.5 μmol, 37.7% yield, 98.5% purity) as a pink solid.

MS (ESI) m/z: 963.8 [M+H]⁺.

¹H NMR (400 MHz, DMSO-d₆) δ=13.12-12.53 (m, 2H), 10.88 (s, 1H), 10.13-9.65 (m, 1H), 8.13 (s, 1H), 8.02 (d, J=4.0 Hz, 2H), 7.81-7.76 (m, 1H), 7.62 (t, J=7.6 Hz, 2H), 7.49-7.43 (m, 3H), 7.40-7.32 (m, 2H), 7.20 (d, J=8.4 Hz, 1H), 7.10 (t, J=8.0 Hz 1H), 6.97 (d, J=8.4 Hz, 1H), 6.91 (d, J=8.0 Hz, 1H), 6.65 (d, J=7.6 Hz, 1H), 4.98 (s, 2H), 4.32 (dd, J=4.8, 9.6 Hz, 1H), 4.09 (s, J=3.2 Hz, 2H), 3.92 (s, 5H), 3.79 (t, J=4.0 Hz, 2H), 3.63-3.43 (m, 3H), 3.03 (t, J=5.6 Hz, 2H), 2.85-2.72 (m, 2H), 2.67-2.60 (m, 3H), 2.36-2.28 (m, 2H), 2.19-2.13 (m, 1H), 1.91 (s, 5H), 1.74-1.54 (m, 2H)

### Example 174. Preparation of Compound 243

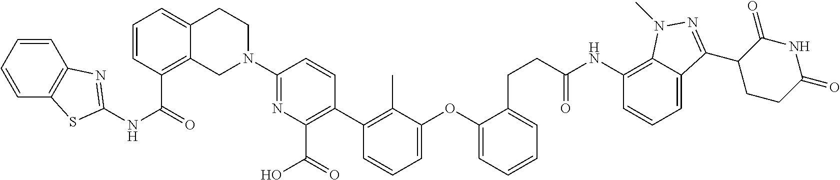 [SMILES: Cc1c(Oc2ccccc2CCC(=O)Nc2cccc3c(C4CCC(=O)NC4=O)nn(C)c23)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(2-(3-((3-(2,6-dioxopiperidin-3-yl)-1-methyl-1H-indazol-7-yl)amino)-3-oxopropyl)phenoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(2-(3-((3-(2,6-dioxopiperidin-3-yl)-1-methyl-1H-indazol-7-yl)amino)-3-oxopropyl)phenoxy)-2-methylphenyl)picolinate

To a solution of 3-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)phenyl)propanoic acid (90 mg, 121.48 μmol, 1 equiv.) in DMF (1 mL) was added HATU (78.52 mg, 206.52 μmol, 1.7 equiv.), DIEA (47.10 mg, 364.44 μmol, 63.48 μL, 3 equiv.), and 3-(7-amino-1-methyl-1H-indazol-3-yl)piperidine-2,6-dione (34.51 mg, 133.63 μmol, 1.1 equiv.). The mixture was stirred at 40° C. for 1 hour. The reaction was added to H₂O (5 mL) and filtered to give tert-butyl 6-(8-(benzo[d]thiazol-2-ylcarbamoyl)-3,4-dihydroisoquinolin-2(1H)-yl)-3-(3-(2-(3-((3-(2,6-dioxopiperidin-3-yl)-1-methyl-1H-indazol-7-yl)amino)-3-oxopropyl)phenoxy)-2-methylphenyl)picolinate (103 mg, crude) as a yellow solid.

MS (ESI) m/z: 981.9 [M+H]⁺

### Step B. Procedure for Preparation of 6-(8-(benzo[d]thiazol-2-ylcarbamoyl)-3,4-dihydroisoquinolin-2(1H)-yl)-3-(3-(2-(3-((3-(2,6-dioxopiperidin-3-yl)-1-methyl-1H-indazol-7-yl)amino)-3-oxopropyl)phenoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(2-(3-((3-(2,6-dioxopiperidin-3-yl)-1-methyl-1H-indazol-7-yl)amino)-3-oxopropyl)phenoxy)-2-methylphenyl)picolinate (103 mg, 104.98 μmol, 1 equiv.) in DCM (0.5 mL) was added TFA (770.00 mg, 6.75 mmol, 0.5 mL, 64.33 equiv.). The mixture was stirred at 25° C. for 12 hours. The mixture was concentrated under reduced pressure to remove DCM and diluted with DMF (2 mL). The resulting mixture was purified by prep-HPLC to give 6-(8-(benzo[d]thiazol-2-ylcarbamoyl)-3,4-dihydroisoquinolin-2(1H)-yl)-3-(3-(2-(3-((3-(2,6-dioxopiperidin-3-yl)-1-methyl-1H-indazol-7-yl)amino)-3-oxopropyl)phenoxy)-2-methylphenyl)picolinic acid (26.4 mg, 27.6 μmol, 26.3% yield, 96.7% purity) as a yellow solid.

MS (ESI) m/z: 925.8 [M+H]⁺

¹H NMR (400 MHz, DMSO-d₆) δ=12.87 (s, 1H), 12.79-12.56 (m, 1H), 10.89 (s, 1H), 9.89 (s, 1H), 8.04 (d, J=8.0 Hz, 1H), 7.79 (d, J=7.6 Hz, 1H), 7.62 (d, J=6.8 Hz, 1H), 7.58 (d, J=7.6 Hz, 1H), 7.54 (d, J=8.8 Hz, 1H), 7.49-7.43 (m, 2H), 7.40-7.33 (m, 3H), 7.21-7.15 (m, 2H), 7.09-7.00 (m, 4H), 6.89 (d, J=7.6 Hz, 1H), 6.83 (d, J=8.0 Hz, 1H), 6.61 (d, J=8.0 Hz, 1H), 5.00 (s, 2H), 4.39-4.32 (m, 1H), 3.94 (t, J=5.2 Hz, 2H), 3.86 (s, 3H), 3.06-3.00 (m, 4H), 3.06-3.00 (m, 2H), 2.61 (d, J=5.2 Hz, 2H), 2.57 (s, 1H), 2.19-2.12 (m, 1H), 2.00-1.94 (m, 3H)

### Example 175. Preparation of Compound 244

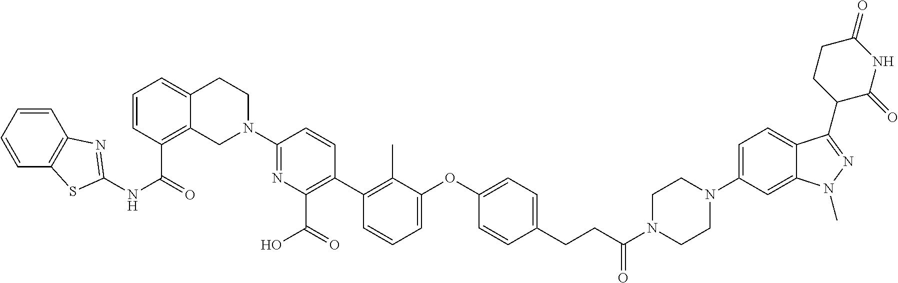 [SMILES: Cc1c(Oc2ccc(CCC(=O)N3CCN(c4ccc5c(C6CCC(=O)NC6=O)nn(C)c5c4)CC3)cc2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(4-(3-(4-(3-(2,6-dioxopiperidin-3-yl)-1-methyl-1H-indazol-6-yl)piperazin-1-yl)-3-oxopropyl)phenoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(4-(3-methoxy-3-oxopropyl)phenoxy)-2-methylphenyl)picolinate

A mixture of methyl 3-(4-(3-bromo-2-methylphenoxy)phenyl)propanoate (300 mg, 859.06 μmol, 1 equiv.) and tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (631.46 mg, 1.03 mmol, 1.2 equiv.), K₂CO₃ (1.5 M, 859.06 μL, 1.5 equiv.) in dioxane (3 mL) was added Ad₂nBuP Pd G₃ (cataCXium® A Pd G₃) (125.13 mg, 171.81 μmol, 0.2 equiv.). The mixture was purged with N₂ and stirred at 80° C. for 3 hours under N₂ atmosphere. The reaction mixture was partitioned between H₂O (4 mL) and ethyl acetate (4 mL). The organic phase was separated, washed with aqueous NaCl (1 mL×3), dried over with Na₂SO₄, filtered, and concentrated under reduced pressure. The residue was purified by flash silica gel chromatography (Eluent of 0˜29% ethyl acetate/petroleum ether) to give tert-butyl 6-(8-(benzo[d]thiazol-2-ylcarbamoyl)-3,4-dihydroisoquinolin-2(1H)-yl)-3-(3-(4-(3-methoxy-3-oxopropyl)phenoxy)-2-methylphenyl)picolinate (377 mg, 464.4 μmol, 54.0% yield, 93% purity) as a yellow solid.

MS (ESI) m/z: 755.4 [M+H]⁺.

### Step B. Procedure for Preparation of 3-(4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)phenyl)propanoic acid

A mixture of tert-butyl 6-(8-(benzo[d]thiazol-2-ylcarbamoyl)-3,4-dihydroisoquinolin-2(1H)-yl)-3-(3-(4-(3-methoxy-3-oxopropyl)phenoxy)-2-methylphenyl)picolinate (377 mg, 499.41 μmol, 1 equiv.) and LiOH·H₂O (1 M, 1.50 mL, 3 equiv.) in THE (4 mL) and H₂O (1 ml) was stirred at 25° C. for 2 hours. The mixture was concentrated and diluted with H₂O (4 mL), and then the pH of the mixture was adjusted to 3 with 1 M HCl. Then the mixture was filtered, and the filtrate was concentrated to give 3-(4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)phenyl)propanoic acid (333 mg, crude) as a yellow solid.

MS (ESI) m/z: 741.5 [M+H]⁺.

### Step C. Procedure for Preparation of tert-butyl 6-(8-(benzo[d]thiazol-2-ylcarbamoyl)-3,4-dihydroisoquinolin-2(1H)-yl)-3-(3-(4-(3-(4-(3-(2,6-dioxopiperidin-3-yl)-1-methyl-1H-indazol-6-yl)piperazin-1-yl)-3-oxopropyl)phenoxy)-2-methylphenyl)picolinate

To a mixture of 3-(4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)phenyl)propanoic acid (90 mg, 121.48 μmol, 1 equiv.), HATU (69.29 mg, 182.22 μmol, 1.5 equiv.), and DIEA (62.80 mg, 485.92 μmol, 84.64 μL, 4 equiv.) in DMF (1.5 mL) was added 3-(1-methyl-6-(piperazin-1-yl)-1H-indazol-3-yl)piperidin-2-one (47.72 mg, 145.78 μmol, 1.2 equiv.). The mixture was then stirred at 25° C. for 2 hours. To the reaction mixture was added water (5 mL), and the resulting mixture was filtered. The filter cake was washed with CH₂Cl₂ (5 mL) and CH₃OH (5 mL) and concentrated under reduced pressure to give tert-butyl 6-(8-(benzo[d]thiazol-2-ylcarbamoyl)-3,4-dihydroisoquinolin-2(1H)-yl)-3-(3-(4-(3-(4-(3-(2,6-dioxopiperidin-3-yl)-1-methyl-1H-indazol-6-yl)piperazin-1-yl)-3-oxopropyl)phenoxy)-2-methylphenyl)picolinate (100 mg, crude) as a white solid.

MS (ESI) m/z: 1050.7 [M+H]⁺.

### Step D. Procedure for Preparation 6-(8-(benzo[d]thiazol-2-ylcarbamoyl)-3,4-dihydroisoquinolin-2(1H)-yl)-3-(3-(4-(3-(4-(3-(2,6-dioxopiperidin-3-yl)-1-methyl-1H-indazol-6-yl)piperazin-1-yl)-3-oxopropyl)phenoxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(3-(4-(3-(4-(3-(2,6-dioxopiperidin-3-yl)-1-methyl-1H-indazol-6-yl)piperazin-1-yl)-3-oxopropyl)phenoxy)-2-methylphenyl)picolinate (100 mg, 95.22 μmol, 1 equiv.) in TFA (1 mL) and DCM (1 mL) was stirred at 40° C. for 67 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(4-(3-(4-(3-(2,6-dioxopiperidin-3-yl)-1-methyl-1H-indazol-6-yl)piperazin-1-yl)-3-oxopropyl)phenoxy)-2-methylphenyl)picolinic acid (39.5 mg, 38.7 μmol, 40.6% yield, 97.2% purity) as a white solid.

MS (ESI) m/z: 994.6 [M+H]⁺.

¹H NMR (400 MHz, DMSO-d₆) δ=12.86 (s, 1H), 12.74-12.57 (m, 1H), 10.84 (s, 1H), 8.03 (d, J=8.0 Hz, 1H), 7.79 (d, J=8.4 Hz, 1H), 7.62 (d, J=7.6 Hz, 1H), 7.52 (d, J=8.8 Hz, 2H), 7.48-7.42 (m, 2H), 7.36 (td, J=7.6, 10.8 Hz, 2H), 7.22 (d, J=8.8 Hz, 2H), 7.11 (t, J=7.6 Hz, 1H), 7.00 (d, J=8.8 Hz, 1H), 6.92 (d, J=9.2 Hz, 1H), 6.88-6.84 (m, 2H), 6.82-6.75 (m, 3H), 5.00 (s, 2H), 4.25 (dd, J=5.2, 9.2 Hz, 1H), 3.93 (t, J=5.6 Hz, 2H), 3.88 (s, 3H), 3.61 (d, J=17.6 Hz, 4H), 3.14 (d, J=4.0 Hz, 4H), 3.03 (t, J=5.6 Hz, 2H), 2.85-2.78 (m, 2H), 2.69-2.65 (m, 2H), 2.60 (d, J=5.6 Hz, 2H), 2.32-2.23 (m, 1H), 2.15 (s, 1H), 1.86 (s, 3H)

### Example 176. Preparation of Compound 247

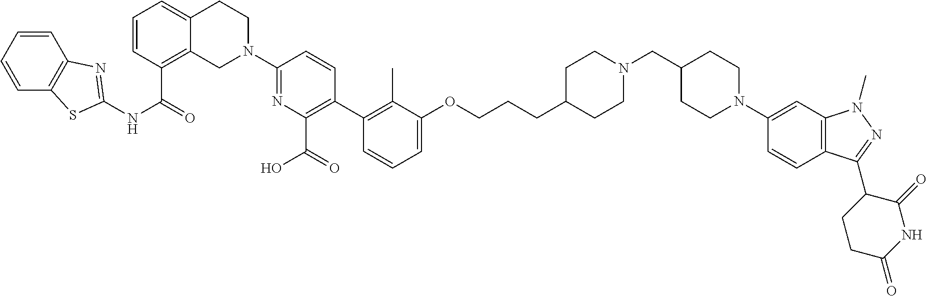 [SMILES: Cc1c(OCCCC2CCN(CC3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[1-[[1-[3-(2,6-dioxo-3-piperidyl)-1-methyl-indazol-6-yl]-4-piperidyl]methyl]-4-piperidyl]propoxy]-2-methyl-phenyl]pyridine-2-carboxylic acid

### Step A. Procedure for Preparation of 6-[8-(1,3-benzothiazol-2-ylcarbamoyl)-3,4-dihydro-1H-isoquinolin-2-yl]-3-[2-methyl-3-[3-(4-piperidyl)propoxy]phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[3-[1-(2,2-diethoxyethyl)-4-piperidyl]propoxy]-2-methyl-phenyl]pyridine-2-carboxylate (1 g, 1.20 mmol, 1.0 equiv.) in HCOOH (10 mL) was stirred at 100° C. for 2 hours. The reaction mixture was concentrated under reduced pressure to give 6-[8-(1,3-benzothiazol-2-ylcarbamoyl)-3,4-dihydro-1H-isoquinolin-2-yl]-3-[2-methyl-3-[3-(4-piperidyl)propoxy]phenyl]pyridine-2-carboxylic acid (1 g, crude) as a yellow oil, which was used in the next step without further purification.

MS (ESI) m/z: 662.5 [M+H]⁺.

### Step B. Procedure for Preparation of 3-[6-[4-(dimethoxymethyl)-1-piperidyl]-1-methyl-indazol-3-yl]piperidine-2,6-dione

A mixture of 3-(2,6-dibenzyloxy-3-pyridyl)-7-[4-(dimethoxymethyl)-1-piperidyl]-1-methyl-indazole (800 mg, 1.38 mmol, 1.0 equiv.), Pd/C (500 mg, 138.24 μmol, 10% purity, 0.1 equiv.), Pd(OH)₂ (500 mg, 3.56 mmol, 2.58 equiv.) and in THE (5 mL) and EtOH (5 mL) was degassed, purged with H₂ (50 Psi), and stirred at 25° C. for 12 hours under H₂ (50 Psi) atmosphere. The reaction mixture was filtered, and the filter cake was immersed with MeOH (30 ml). The filtrate was concentrated under reduced pressure to give 3-[6-[4-(dimethoxymethyl)-1-piperidyl]-1-methyl-indazol-3-yl]piperidine-2,6-dione (380 mg, 677.1 μmol, 48.9% yield, 71.3% purity) as a brown solid.

MS (ESI) m/z: 401.1 [M+H]⁺.

### Step C. Procedure for Preparation of 1-[3-(2,6-dioxo-3-piperidyl)-1-methyl-indazol-6-yl]piperidine-4-carbaldehyde

A mixture of 3-[6-[4-(dimethoxymethyl)-1-piperidyl]-1-methyl-indazol-3-yl]piperidine-2,6-dione (100 mg, 249.71 μmol, 1.0 equiv.) in HCOOH (1 mL) was degassed and purged with N₂ and stirred at 90° C. for 2 hours under N₂ atmosphere. The residue was concentrated to give 1-[3-(2,6-dioxo-3-piperidyl)-1-methyl-indazol-6-yl]piperidine-4-carbaldehyde (50 mg, crude) as a brown oil.

MS (ESI) m/z: 387.1 [M+H]⁺.

### Step D. Procedure for Preparation of 6-[8-(1,3-benzothiazol-2-ylcarbamoyl)-3,4-dihydro-1H-isoquinolin-2-yl]-3-[3-[3-[1-[[1-[3-(2,6-dioxo-3-piperidyl)-1-methyl-indazol-6-yl]-4-piperidyl]methyl]-4-piperidyl]propoxy]-2-methyl-phenyl]pyridine-2-carboxylic acid

To a solution of 6-[8-(1,3-benzothiazol-2-ylcarbamoyl)-3,4-dihydro-1H-isoquinolin-2-yl]-3-[2-methyl-3-[3-(4-piperidyl)propoxy]phenyl]pyridine-2-carboxylic acid (100 mg, 151.10 μmol, 1.0 equiv.) in DCM (2 mL) and IPA (2 mL) was added 1-[3-(2,6-dioxo-3-piperidyl)-1-methyl-indazol-6-yl]piperidine-4-carbaldehyde (64.26 mg, 181.32 μmol, 1.2 equiv.) at 25° C. for 16 hours. After addition, the mixture was stirred at this temperature, and then NaBH(OAc)₃ (96.07 mg, 453.30 μmol, 3.0 equiv.) was added at 0° C. The mixture was stirred at 25° C. for 1 hour, after which it was concentrated. The crude product was purified by prep-HPLC to give 6-[8-(1,3-benzothiazol-2-ylcarbamoyl)-3,4-dihydro-1H-isoquinolin-2-yl]-3-[3-[3-[1-[[1-[3-(2,6-dioxo-3-piperidyl)-1-methyl-indazol-6-yl]-4-piperidyl]methyl]-4-piperidyl]propoxy]-2-methyl-phenyl]pyridine-2-carboxylic acid (21.7 mg, 21.1 μmol, 14.0% yield, 97.2% purity) as a yellow solid.

MS (ESI) m/z: 500.9 [M/2+H]⁺.

1H NMR (400 MHz, DMSO-d₆) δ=10.84 (s, 1H), 8.18 (s, 1H), 8.01-8.01 (m, 1H), 8.01 (d, J=8.0 Hz, 1H), 7.78 (d, J=8.0 Hz, 1H), 7.62 (d, J=8.0 Hz, 1H), 7.47-7.42 (m, 3H), 7.40-7.32 (m, 3H), 7.10-7.05 (m, 1H), 6.93-6.85 (m, 3H), 6.81 (s, 1H), 6.64 (d, J=7.6 Hz, 1H), 4.97 (s, 2H), 4.24 (dd, J=5.2, 9.2 Hz, 1H), 3.92 (d, J=5.6 Hz, 2H), 3.87 (s, 3H), 3.78-3.74 (m, 2H), 3.02 (t, J=5.2 Hz, 2H), 2.92-2.80 (m, 4H), 2.74-2.69 (m, 2H), 2.68-2.64 (m, 1H), 2.63-2.55 (m, 2H), 2.30-2.23 (m, 2H), 2.19-2.12 (m, 4H), 1.90 (s, 3H), 1.79-1.73 (m, 4H), 1.70-1.59 (m, 3H), 1.39-1.34 (m, 2H), 1.29-1.19 (m, 4H)

### Example 177. Preparation of Compound 251a

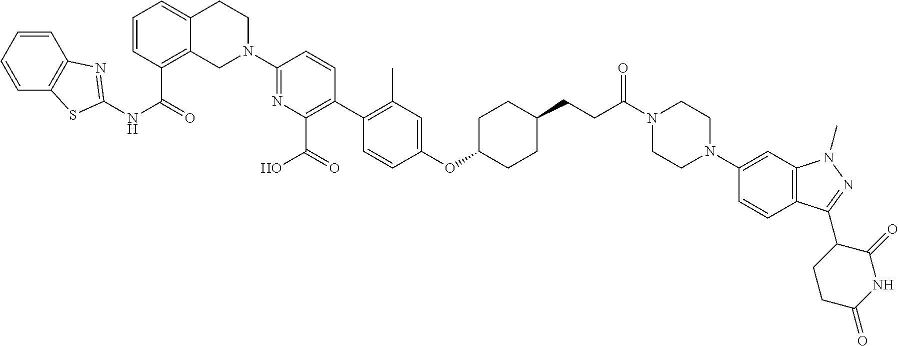 [SMILES: Cc1cc(O[C@H]2CC[C@H](CCC(=O)N3CCN(c4ccc5c(C6CCC(=O)NC6=O)nn(C)c5c4)CC3)CC2)ccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(4-(((1r,4r)-4-(3-(4-(3-(2,6-dioxopiperidin-3-yl)-1-methyl-1H-indazol-6-yl)piperazin-1-yl)-3-oxopropyl)cyclohexyl)oxy)-2-methylphenyl)picolinic acid

### Step A. Procedure for Preparation of (1r,4r)-methyl 4-(4-bromo-3-methylphenoxy)cyclohexanecarboxylate

To a solution of (1r,4r)-methyl 4-hydroxycyclohexanecarboxylate (2.0 g, 12.64 mmol, 1.1 equiv.) in toluene (20 mL) was added 2-(tributyl-λ5-phosphanylidene)acetonitrile (3.33 g, 13.79 mmol, 1.2 equiv.) and 4-bromo-3-methylphenol (2.15 g, 11.49 mmol, 1.0 equiv.). The mixture was stirred at 120° C. for 12 hours. The reaction mixture was concentrated under reduced pressure to remove solvent. The residue was purified by column chromatography (SiO₂, petroleum ether/ethyl acetate=1/0) to give (1r,4r)-methyl 4-(4-bromo-3-methylphenoxy)cyclohexanecarboxylate (2.4 g, crude) as a yellow oil.

### Step B. Procedure for Preparation of ((1r,4r)-4-(4-bromo-3-methylphenoxy)cyclohexyl)methanol

To a solution of (1r,4r)-methyl 4-(4-bromo-3-methylphenoxy)cyclohexanecarboxylate (2.4 g, 7.33 mmol, 1 equiv.) in THF (25 mL) was added LAH (278.38 mg, 7.33 mmol, 1 equiv.). The mixture was stirred at 0° C. for 1 hour under N₂ atmosphere. The reaction mixture was quenched by addition H₂O 0.3 mL, 15% NaOH 0.3 mL and H₂O 0.9 mL, the combined mixture was mixed with Na₂SO₄, then filtered and concentrated under reduced pressure to give a residue. The residue was purified by flash silica gel chromatography (Eluent of 0-20% ethyl acetate/petroleum ether) to give compound ((1r,4r)-4-(4-bromo-3-methylphenoxy)cyclohexyl)methanol (1.3 g, 4.3 mmol, 59.2% yield) as a yellow oil.

¹H NMR (400 MHz) δ=7.40 (d, J=8.8 Hz, 1H), 6.94 (d, J=2.8 Hz, 1H), 6.75-6.67 (m, 1H), 4.43 (t, J=5.6 Hz, 1H), 4.26-4.15 (m, 1H), 3.23 (t, J=5.6 Hz, 2H), 2.28 (s, 3H), 2.08-2.00 (m, 2H), 1.77 (d, J=11.6 Hz, 2H), 1.43-1.33 (m, 1H), 1.33-1.22 (m, 2H), 1.11-0.98 (m, 2H)

### Step C. Procedure for Preparation of (1r,4r)-4-(4-bromo-3-methylphenoxy)cyclohexanecarbaldehyde

To a solution of oxalyl dichloride (1.10 g, 8.69 mmol, 760.68 μL, 2 equiv.) in DCM (20 mL) was added dropwise a solution of DMSO (1.36 g, 17.38 mmol, 1.36 mL, 4 equiv.) in DCM (2 mL) at −70° C. under N₂ atmosphere. The mixture was stirred at −70° C. for 1 hour. After which time ((1r,4r)-4-(4-bromo-3-methylphenoxy)cyclohexyl)methanol (1.3 g, 4.34 mmol, 1 equiv.) in DCM (2 mL) was added dropwise at −70° C. The solution was stirred for 1 hour at −70° C. Then TEA (2.64 g, 26.07 mmol, 3.63 mL, 6 equiv.) was added into the solution. The solution was stirred at −70° C. for 0.5 hour under N₂ atmosphere. DCM (50 mL) and water (50 mL) were added, and the layers were separated. The aqueous layer was extracted with DCM (30 mL×2). The combined organic extracts were dried over anhydrous sodium sulfate, filtered, and concentrated under vacuum to give (1r,4r)-4-(4-bromo-3-methylphenoxy)cyclohexanecarbaldehyde (1.2 g, 4.04 mmol, 92.9% yield) as a yellow oil.

### Step D. Procedure for Preparation of (E)-ethyl 3-((1r,4r)-4-(4-bromo-3-methylphenoxy)cyclohexyl)acrylate

To a solution of ethyl 2-(diethoxyphosphoryl)acetate (1.81 g, 8.08 mmol, 1.60 mL, 2.0 equiv.) in THE (15 mL) was added NaH (339.15 mg, 8.48 mmol, 60% purity, 2.1 equiv.). The mixture was stirred at 0° C. for 1 hour. Then (1r,4r)-4-(4-bromo-3-methylphenoxy)cyclohexanecarbaldehyde (1.2 g, 4.04 mmol, 1 equiv.) was added into the mixture, which was stirred at 25° C. for 0.5 hour under N₂. The reaction mixture was quenched by addition water (10 mL) at 0° C., and then diluted with water (10 mL) and extracted with ethyl acetate (30 mL×3). The combined organic layers were washed with water (10 mL×3), filtered, and concentrated under reduced pressure to give a residue. The residue was purified by column chromatography (SiO₂, petroleum ether/ethyl acetate=0/1 to 10/1) to give (E)-ethyl 3-((1r,4r)-4-(4-bromo-3-methylphenoxy)cyclohexyl)acrylate (1 g, 2.7 mmol, 67.4% yield) as a white solid.

¹H NMR (400 MHz) δ=7.41 (d, J=8.8 Hz, 1H), 6.96 (d, J=2.8 Hz, 1H), 6.76-6.70 (m, 1H), 6.16-6.07 (m, 1H), 5.74 (d, J=11.6 Hz, 1H), 4.32-4.22 (m, 1H), 4.14-4.06 (m, 2H), 3.25-3.15 (m, 1H), 2.29 (s, 3H), 2.12-2.02 (m, 2H), 1.77-1.65 (m, 2H), 1.39-1.28 (m, 4H), 1.21 (t, J=7.2 Hz, 3H)

### Step E. Procedure for Preparation of ethyl 3-((1r,4r)-4-(4-bromo-3-methylphenoxy)cyclohexyl)propanoate

To a solution of (E)-ethyl 3-((1r,4r)-4-(4-bromo-3-methylphenoxy)cyclohexyl)acrylate (1 g, 2.72 mmol, 1 equiv.) in EtOH (10 mL) was added PtO₂ (61.83 mg, 272.27 μmol, 0.1 equiv.) under H₂ atmosphere (15 psi). The mixture was stirred at 25° C. for 12 hours. The reaction mixture was filtered and concentrated under reduced pressure to give a filter cake which was rinsed with a little EtOH. The filter liquor was collected and concentrated under reduced pressure to give a residue. The residue was purified by column chromatography (SiO₂, petroleum ether/ethyl acetate=20/1 to 10/1) to give ethyl 3-((1r,4r)-4-(4-bromo-3-methylphenoxy)cyclohexyl)propanoate (700 mg, 1.8 mmol, 65.2% yield, 93.7% purity) as a colorless oil. 1H NMR (400 MHz) δ=7.40 (d, J=8.4 Hz, 1H), 6.94 (d, J=2.8 Hz, 1H), 6.74-6.67 (m, 1H), 4.28-4.16 (m, 1H), 4.09-4.00 (m, 2H), 2.34-2.22 (m, 5H), 2.02 (d, J=10.0 Hz, 2H), 1.74 (d, J=12.0 Hz, 2H), 1.51-1.40 (m, 2H), 1.34-1.22 (m, 3H), 1.21-1.15 (m, 3H), 1.11-0.98 (m, 2H)

### Step F. Procedure for Preparation of tert-butyl 6-(8-(benzo[d]thiazol-2-ylcarbamoyl)-3,4-dihydroisoquinolin-2(1H)-yl)-3-(4-(((1r,4r)-4-(3-ethoxy-3-oxopropyl)cyclohexyl)oxy)-2-methylphenyl)picolinate

To a solution of tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (331.74 mg, 487.42 μmol, 90% purity, 1.2 equiv.) in dioxane (3 mL) was added ethyl 3-((1r,4r)-4-(4-bromo-3-methylphenoxy)cyclohexyl)propanoate (150 mg, 406.18 μmol, 1.0 equiv.), KF (1.5 M, 812.36 μL, 3.0 equiv.), and [2-(2-aminophenyl)phenyl]palladium(1+); bis(1-adamantyl)-butyl-phosphane; methanesulfonate (29.58 mg, 40.62 μmol, 0.1 equiv.). The mixture was stirred at 100° C. for 1 hour. The reaction mixture was concentrated under reduced pressure to remove solvent. The residue was purified by flash silica gel chromatography (Eluent of 0-30% ethyl acetate/petroleum ether) to give compound tert-butyl 6-(8-(benzo[d]thiazol-2-ylcarbamoyl)-3,4-dihydroisoquinolin-2(1H)-yl)-3-(4-(((1r,4r)-4-(3-ethoxy-3-oxopropyl)cyclohexyl)oxy)-2-methylphenyl)picolinate (360 mg, 371.6 μmol, 91.4% yield, 80% purity) as a yellow solid.

MS (ESI) m/z: 775.5 [M+H]⁺

### Step G. Procedure for Preparation of 3-((1r,4r)-4-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)cyclohexyl)propanoic acid

To a solution of tert-butyl 6-(8-(benzo[d]thiazol-2-ylcarbamoyl)-3,4-dihydroisoquinolin-2(1H)-yl)-3-(4-(((1r,4r)-4-(3-ethoxy-3-oxopropyl)cyclohexyl)oxy)-2-methylphenyl)picolinate (360 mg, 371.63 μmol, 80% purity, 1.0 equiv.) in THE (4 mL) and H₂O (1.3 mL) was added LiOH·H₂O (46.78 mg, 1.11 mmol, 3.0 equiv.). The mixture was stirred at 25° C. for 12 hours. The reaction mixture was concentrated under reduced pressure to remove solvent, to the residue was added H₂O (5 mL). The pH of the mixture was adjusted to 4 with 1M HCl. Then the reaction mixture was filtered and concentrated under reduced pressure to give 3-((1r,4r)-4-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)cyclohexyl)propanoic acid (300 mg, 313.2 μmol, 84.3% yield, 78% purity) as a yellow solid.

MS (ESI) m/z: 747.2 [M+H]⁺

### Step H. Procedure for Preparation of tert-butyl 6-(8-(benzo[d]thiazol-2-ylcarbamoyl)-3,4-dihydroisoquinolin-2(1H)-yl)-3-(4-(((1r,4r)-4-(3-(4-(3-(2,6-dioxopiperidin-3-yl)-1-methyl-1H-indazol-6-yl)piperazin-1-yl)-3-oxopropyl)cyclohexyl)oxy)-2-methylphenyl)picolinate

To a solution of 3-((1r,4r)-4-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)cyclohexyl)propanoic acid (100 mg, 133.88 μmol, 1.0 equiv.) in pyridine (1 mL) was added EDCI (33.37 mg, 174.05 μmol, 1.3 equiv.) and 3-(1-methyl-6-(piperazin-1-yl)-1H-indazol-3-yl)piperidine-2,6-dione (58.46 mg, 160.66 μmol, 1.2 equiv.). The mixture was stirred at 40° C. for 4 hours. The reaction mixture was concentrated under reduced pressure to remove solvent. The residue was purified by column chromatography (SiO₂, petroleum ether/ethyl acetate=4/1 to 0/1, DCM:MeOH=10:1) to give tert-butyl 6-(8-(benzo[d]thiazol-2-ylcarbamoyl)-3,4-dihydroisoquinolin-2(1H)-yl)-3-(4-(((1r,4r)-4-(3-(4-(3-(2,6-dioxopiperidin-3-yl)-1-methyl-1H-indazol-6-yl)piperazin-1-yl)-3-oxopropyl)cyclohexyl)oxy)-2-methylphenyl)picolinate (90 mg, 85.2 μmol, 63.6% yield) as a yellow solid.

MS (ESI) m/z: 1056.4 [M+H]⁺

### Step I. Procedure for preparation of 6-(8-(benzo[d]thiazol-2-ylcarbamoyl)-3,4-dihydroisoquinolin-2(1H)-yl)-3-(4-(((1r,4r)-4-(3-(4-(3-(2,6-dioxopiperidin-3-yl)-1-methyl-1H-indazol-6-yl)piperazin-1-yl)-3-oxopropyl)cyclohexyl)oxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(4-(((1r,4r)-4-(3-(4-(3-(2,6-dioxopiperidin-3-yl)-1-methyl-1H-indazol-6-yl)piperazin-1-yl)-3-oxopropyl)cyclohexyl)oxy)-2-methylphenyl)picolinate (90 mg, 85.20 μmol, 1.0 equiv.) in DCM (1 mL) was added TFA (1.54 g, 13.51 mmol, 1 mL, 158.51 equiv.). The mixture was stirred at 25° C. for 12 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(4-(((1r,4r)-4-(3-(4-(3-(2,6-dioxopiperidin-3-yl)-1-methyl-1H-indazol-6-yl)piperazin-1-yl)-3-oxopropyl)cyclohexyl)oxy)-2-methylphenyl)picolinic acid (20.34 mg, 18.6 μmol, 21.8% yield, 91.6% purity) as an off-white solid.

MS (ESI) m/z: 1000.4 [M+H]⁺

¹H NMR (400 MHz, DMSO-d₆) δ=12.86 (s, 1H), 12.70-12.48 (m, 1H), 10.85 (s, 1H), 8.03 (d, J=7.6 Hz, 1H), 7.79 (d, J=7.6 Hz, 1H), 7.62 (d, J=7.2 Hz, 1H), 7.53 (d, J=8.8 Hz, 1H), 7.49-7.42 (m, 3H), 7.39-7.32 (m, 2H), 6.95 (d, J=8.8 Hz, 2H), 6.91-6.86 (m, 2H), 6.78 (d, J=2.0 Hz, 1H), 6.73-6.68 (m, 1H), 4.97 (s, 2H), 4.29-4.25 (m, 1H), 4.24-4.17 (m, 1H), 3.93-3.86 (m, 5H), 3.63 (s, 2H), 3.23 (s, 2H), 3.18 (s, 2H), 3.02 (t, J=6.0 Hz, 2H), 2.67-2.57 (m, 2H), 2.41-2.37 (m, 2H), 2.34-2.23 (m, 2H), 2.21-2.13 (m, 1H), 2.10-2.03 (m, 2H), 2.01 (s, 3H), 1.86-1.78 (m, 2H), 1.49-1.41 (m, 2H), 1.35-1.22 (m, 4H), 1.15-1.04 (m, 2H)

### Example 178. Preparation of Compound 256

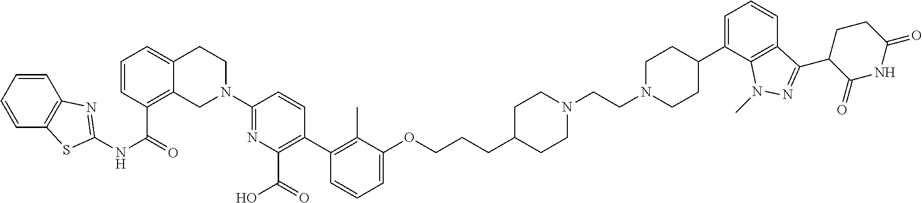 [SMILES: Cc1c(OCCCC2CCN(CCN3CCC(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[1-[2-[4-[3-(2,6-dioxo-3-piperidyl)-1-methyl-indazol-7-yl]-1-piperidyl]ethyl]-4-piperidyl]propoxy]-2-methyl-phenyl]pyridine-2-carboxylic acid

### Step A. Procedure for Preparation of 6-[8-(1,3-benzothiazol-2-ylcarbamoyl)-3,4-dihydro-1H-isoquinolin-2-yl]-3-[2-methyl-3-[3-[1-(2-oxoethyl)-4-piperidyl]propoxy]phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[3-[1-(2,2-diethoxyethyl)-4-piperidyl]propoxy]-2-methyl-phenyl]pyridine-2-carboxylate (200 mg, 239.79 μmol, 1.0 equiv.) in HCOOH (2 mL) was stirred at 100° C. for 2 hours. The reaction mixture was concentrated under reduced pressure to give 6-[8-(1,3-benzothiazol-2-ylcarbamoyl)-3,4-dihydro-1H-isoquinolin-2-yl]-3-[2-methyl-3-[3-[1-(2-oxoethyl)-4-piperidyl]propoxy]phenyl]pyridine-2-carboxylic acid (200 mg, crude) as a black oil.

MS (ESI) m/z: 772.2 [M+H]⁺

### Step B. Procedure for Preparation of 6-[8-(1,3-benzothiazol-2-ylcarbamoyl)-3,4-dihydro-1H-isoquinolin-2-yl]-3-[3-[3-[1-[2-[4-[3-(2,6-dioxo-3-piperidyl)-1-methyl-indazol-7-yl]-1-piperidyl]ethyl]-4-piperidyl]propoxy]-2-methyl-phenyl]pyridine-2-carboxylic acid

To a solution of NaBH(OAc)₃ (73.74 mg, 347.92 μmol, 3.0 equiv.) and 3-[1-methyl-7-(4-piperidyl)indazol-3-yl]piperidine-2,6-dione (37.85 mg, 115.97 μmol, 1 equiv.) in DCM (1 mL) was added 6-[8-(1,3-benzothiazol-2-ylcarbamoyl)-3,4-dihydro-1H-isoquinolin-2-yl]-3-[2-methyl-3-[3-[1-(2-oxoethyl)-4-piperidyl]propoxy]phenyl]pyridine-2-carboxylic acid (81.63 mg, 115.97 μmol, 1.0 equiv.) dropwise at 0° C. The mixture was stirred at 25° C. for 12 hours. The reaction mixture was filtered and concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[3-[1-[2-[4-[3-(2,6-dioxo-3-piperidyl)-1-methyl-indazol-7-yl]-1-piperidyl]ethyl]-4-piperidyl]propoxy]-2-methyl-phenyl]pyridine-2-carboxylic acid (17.1 mg, 18.8 μmol, 16.2% yield, 95.3% purity) as a white solid.

MS (ESI) m/z: 1014.6 [M+H]⁺

¹H NMR (400 MHz, DMSO-d₆) δ=10.89 (s, 1H), 8.15 (s, 1H), 8.02 (d, J=8.0 Hz, 1H), 7.78 (d, J=8.0 Hz, 1H), 7.62 (d, J=6.4 Hz, 1H), 7.54 (d, J=8.0 Hz, 1H), 7.49-7.30 (m, 5H), 7.23 (d, J=7.2 Hz, 1H), 7.12-7.03 (m, 2H), 6.94-6.84 (m, 2H), 6.64 (d, J=7.6 Hz, 1H), 4.96 (s, 2H), 4.42-4.31 (m, 1H), 4.20 (s, 2H), 3.98 (s, 3H), 3.89 (s, 2H), 3.20-3.09 (m, 6H), 2.99 (s, 2H), 2.84-2.73 (m, 3H), 2.71-2.62 (m, 4H), 2.44 (s, 2H), 2.38-2.29 (m, 4H), 2.20-2.13 (m, 1H), 1.93-1.83 (m, 5H), 1.77-1.65 (m, 4H), 1.37 (s, 3H), 1.23 (s, 1H).

### Example 179. Preparation of Compound 257

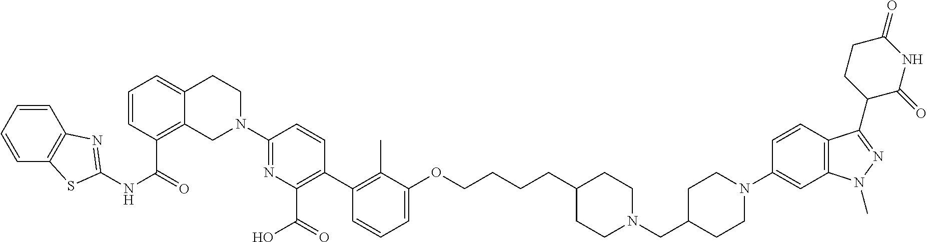 [SMILES: Cc1c(OCCCCC2CCN(CC3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[4-[1-[[1-[3-(2,6-dioxo-3-piperidyl)-1-methyl-indazol-6-yl]-4-piperidyl]methyl]-4-piperidyl]butoxy]-2-methyl-phenyl]pyridine-2-carboxylic acid

### Step A. Procedure for Preparation of tert-butyl 4-[4-(3-bromo-2-methyl-phenoxy)butyl]piperidine-1-carboxylate

A mixture of 3-bromo-2-methyl-phenol (650 mg, 3.48 mmol, 1.0 equiv.), tert-butyl 4-(4-hydroxybutyl)piperidine-1-carboxylate (1.07 g, 4.17 mmol, 1.2 equiv.), and 2-(tributyl-λ5-phosphanylidene)acetonitrile (1.26 g, 5.21 mmol, 1.5 equiv.) in toluene (8 mL) was degassed, purged with N₂, and stirred at 120° C. for 2 hours under N₂ atmosphere. The residue was concentrated, and the crude product was purified by reverse-phase HPLC to give tert-butyl 4-[4-(3-bromo-2-methyl-phenoxy)butyl]piperidine-1-carboxylate (1.1 g, 2.5 mmol, 74.2% yield) as a brown oil.

MS (ESI) m/z: 335.1 [M+H]⁺.

1H NMR (400 MHz, DMSO-d6) δ=7.17-7.12 (m, 1H), 7.11-7.05 (m, 1H), 6.95 (d, J=8.0 Hz, 1H), 3.97 (t, J=6.4 Hz, 2H), 3.91 (d, J=12.0 Hz, 2H), 2.66 (d, J=2.0 Hz, 2H), 2.22 (s, 3H), 1.75-1.67 (m, 2H), 1.62 (d, J=12.0 Hz, 2H), 1.48-1.40 (m, 3H), 1.38 (s, 9H), 1.28-1.22 (m, 2H), 1.02-0.88 (m, 2H).

### Step B. Procedure for Preparation of tert-butyl 6-[8-(1,3-benzothiazol-2-ylcarbamoyl)-3,4-dihydro-1H-isoquinolin-2-yl]-3-[3-[4-(1-tert-butoxycarbonyl-4-piperidyl)butoxy]-2-methyl-phenyl]pyridine-2-carboxylate

tert-Butyl 4-[4-(3-bromo-2-methyl-phenoxy)butyl]piperidine-1-carboxylate (500 mg, 1.17 mmol, 1.0 equiv.), tert-butyl.6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (718.30 mg, 1.17 mmol, 1.0 equiv.) and Ad₂nBuP Pd G₃ (85.40 mg, 117.26 μmol, 0.1 equiv.), and K₂CO₃ (486.20 mg, 3.52 mmol, 3.0 equiv.) were taken up into a microwave tube in dioxane (6 mL) and H₂O (2 mL). The sealed tube was heated at 100° C. for 1 hour under microwave. The mixture was concentrated, and the resulting residue was purified by flash silica gel chromatography (Eluent of 33% ethyl acetate/petroleum ether) to give tert-butyl 6-[8-(1,3-benzothiazol-2-ylcarbamoyl)-3,4-dihydro-1H-isoquinolin-2-yl]-3-[3-[4-(1-tert-butoxycarbonyl-4-piperidyl)butoxy]-2-methyl-phenyl]pyridine-2-carboxylate (700 mg, 841.2 μmol, 71.7% yield) as a brown oil.

MS (ESI) m/z: 832.1 [M+H]⁺.

1H NMR (400 MHz, DMSO-d₆) δ=12.86 (s, 1H), 8.02 (d, J=7.6 Hz, 1H), 7.80-7.75 (m, 1H), 7.60 (d, J=7.6 Hz, 1H), 7.48-7.42 (m, 3H), 7.39-7.34 (m, 2H), 7.12-7.07 (m, 1H), 6.93 (d, J=8.4, 14.1 Hz, 2H), 6.57 (d, J=7.6 Hz, 1H), 4.98 (s, 2H), 4.03 (d, J=7.2 Hz, 3H), 3.03 (t, J=5.6 Hz, 2H), 1.99 (s, 4H), 1.87 (s, 2H), 1.37-1.36 (m, 9H), 1.18 (t, J=7.2 Hz, 5H), 1.07 (s, 8H), 1.00 (s, 9H).

### Step C. Procedure for Preparation of 6-[8-(1,3-benzothiazol-2-ylcarbamoyl)-3,4-dihydro-1H-isoquinolin-2-yl]-3-[2-methyl-3-[4-(4-piperidyl)butoxy]phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[4-(1-tertbutoxycarbonyl-4-piperidyl)butoxy]-2-methyl-phenyl]pyridine-2-carboxylate (130 mg, 156.24 μmol, 1.0 equiv.) in HCOOH (2 mL) was stirred at 90° C. for 2 hours. The mixture was concentrated to provide 6-[8-(1,3-benzothiazol-2-ylcarbamoyl)-3,4-dihydro-1H-isoquinolin-2-yl]-3-[2-methyl-3-[4-(4-piperidyl)butoxy]phenyl]pyridine-2-carboxylic acid (81 mg, 119.8 μmol, 76.7% yield) as a brown oil, which was used in the next step without further purification.

MS (ESI) m/z: 676.2 [M+H]⁺.

### Step D. Procedure for Preparation of 1-[3-(2,6-dioxo-3-piperidyl)-1-methyl-indazol-6-yl]piperidine-4-carbaldehyde

A mixture of 3-[6-[4-(dimethoxymethyl)-1-piperidyl]-1-methyl-indazol-3-yl]piperidine-2,6-dione (100 mg, 249.71 μmol, 1 equiv.) in HCOOH (1 mL) was degassed and purged with N₂ three times. The mixture was then stirred at 90° C. for 2 hours under N₂ atmosphere. The reaction mixture was then concentrated to provide 1-[3-(2,6-dioxo-3-piperidyl)-1-methyl-indazol-6-yl]piperidine-4-carbaldehyde (74 mg, crude) as a brown oil, which was used in the next step without further purification.

MS (ESI) m/z: 373.1 [M+H]⁺.

### Step E. Procedure for Preparation of 6-[8-(1,3-benzothiazol-2-ylcarbamoyl)-3,4-dihydro-1H-isoquinolin-2-yl]-3-[3-[4-[1-[[1-[3-(2,6-dioxo-3-piperidyl)-1-methyl-indazol-6-yl]-4-piperidyl]methyl]-4-piperidyl]butoxy]-2-methyl-phenyl]pyridine-2-carboxylic acid

To a solution of 6-[8-(1,3-benzothiazol-2-ylcarbamoyl)-3,4-dihydro-1H-isoquinolin-2-yl]-3-[2-methyl-3-[4-(4-piperidyl)butoxy]phenyl]pyridine-2-carboxylic acid (103.29 mg, 152.84 μmol, 1 equiv.) in DCM (0.5 mL) and IPA (0.5 mL) was added 1-[3-(2,6-dioxo-3-piperidyl)-1-methyl-indazol-6-yl]piperidine-4-carbaldehyde (65 mg, 183.41 μmol, 1.2 equiv.) at 25° C. for 11 hours. The mixture was stirred at this temperature, and then NaBH(OAc)₃ (97.18 mg, 458.52 μmol, 3 equiv.) was added at 0° C. The resulting mixture was stirred at 25° C. for 1 hour. The reaction mixture was concentrated and purified by reverse-phase HPLC to give 6-[8-(1,3-benzothiazol-2-ylcarbamoyl)-3,4-dihydro-1H-isoquinolin-2-yl]-3-[3-[4-[1-[[1-[3-(2,6-dioxo-3-piperidyl)-1-methyl-indazol-6-yl]-4-piperidyl]methyl]-4-piperidyl]butoxy]-2-methyl-phenyl]pyridine-2-carboxylic acid (37.1 mg, 35.7 μmol, 23.3% yield, 97.4% purity) as an off-white solid.

MS (ESI) m/z: 507.9 [M/2+H]⁺.

1H NMR (400 MHz, DMSO-d₆) δ=10.84 (s, 1H), 8.18 (s, 1H), 8.01 (d, J=8.0 Hz, 1H), 7.78 (d, J=8.0 Hz, 1H), 7.62 (d, J=7.2 Hz, 1H), 7.50-7.30 (m, 6H), 7.12-7.04 (m, 1H), 6.94-6.84 (m, 3H), 6.80 (s, 1H), 6.63 (d, J=7.6 Hz, 1H), 4.96 (s, 2H), 4.24 (dd, J=4.8, 8.8 Hz, 1H), 3.99-3.95 (m, 2H), 3.91 (t, J=5.2 Hz, 2H), 3.87 (s, 3H), 3.79-3.70 (m, 2H), 3.56 (s, 2H), 3.00 (t, J=5.6 Hz, 2H), 2.87 (d, J=10.0 Hz, 2H), 2.74-2.66 (m, 2H), 2.64-2.57 (m, 2H), 2.35-2.24 (m, 2H), 2.20 (d, J=6.4 Hz, 2H), 1.97-1.86 (m, 5H), 1.82-1.77 (m, 2H), 1.74-1.69 (m, 2H), 1.61 (d, J=11.2 Hz, 2H), 1.44 (d, J=4.8 Hz, 2H), 1.31-1.16 (m, 6H)

### Example 180. Preparation of Compound 258

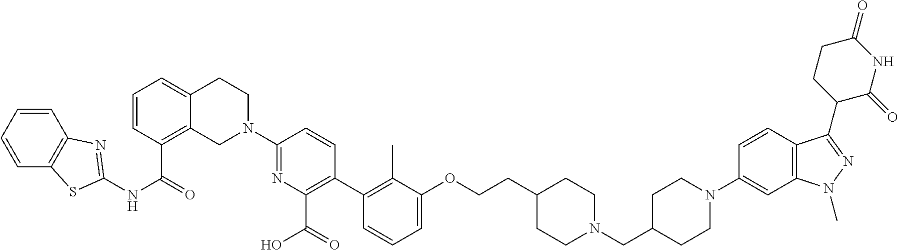 [SMILES: Cc1c(OCCC2CCN(CC3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[2-[1-[[1-[3-(2,6-dioxo-3-piperidyl)-1-methyl-indazol-6-yl]-4-piperidyl]methyl]-4-piperidyl]ethoxy]-2-methyl-phenyl]pyridine-2-carboxylic acid

### Step A. Procedure for Preparation of tert-butyl 4-[2-(3-bromo-2-methyl-phenoxy)ethyl]piperidine-1-carboxylate

To a solution of tert-butyl 4-(2-bromoethyl)piperidine-1-carboxylate (1 g, 3.42 mmol, 1.0 equiv.) and 3-bromo-2-methyl-phenol (704.07 mg, 3.76 mmol, 1.1 equiv.) in CH₃CN (10 mL) was added K₂CO₃ (1.42 g, 10.27 mmol, 3.0 equiv.). The mixture was stirred at 60° C. for 16 hours. The mixture was filtered to give a filtrate, which was concentrated under reduced pressure to give a residue. The residue was purified by flash silica gel chromatography (Eluent of 0˜4% ethyl acetate/petroleum ether) to give tert-butyl 4-[2-(3-bromo-2-methyl-phenoxy)ethyl]piperidine-1-carboxylate (1.2 g, 2.9 mmol, 86.9% yield, 98.8% purity) as a yellow oil.

MS (ESI) m/z: 298.2 [M−100+H]⁺

¹H NMR (400 MHz, CD₃OD) δ=7.13-7.09 (m, 1H), 7.02 (t, J=8.0 Hz, 1H), 6.89 (d, J=8.0 Hz, 1H), 4.10-4.07 (m, 1H), 4.07-4.03 (m, 3H), 2.84-2.69 (m, 2H), 2.28 (s, 3H), 1.79-1.73 (m, 5H), 1.45 (s, 9H), 1.22-1.11 (m, 2H)

### Step B. Procedure for Preparation of tert-butyl 6-[8-(1,3-benzothiazol-2-ylcarbamoyl)-3,4-dihydro-1H-isoquinolin-2-yl]-3-[3-[2-(1-tert-butoxycarbonyl-4-piperidyl)ethoxy]-2-methyl-phenyl]pyridine-2-carboxylate

tert-Butyl 4-[2-(3-bromo-2-methyl-phenoxy)ethyl]piperidine-1-carboxylate (1.18 g, 2.96 mmol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (1.81 g, 2.96 mmol, 1.0 equiv.), KF (516.31 mg, 8.89 mmol, 208.19 μL, 3.0 equiv.), and Ad₂nBuP Pd G₃ (215.74 mg, 296.23 μmol, 0.1 equiv.) were taken up into a microwave tube in dioxane (10 mL) and H₂O (1 mL). The sealed tube was heated at 100° C. for 2 hours under microwave. The mixture was concentrated under reduced pressure to give a residue. The residue was purified by flash silica gel chromatography (Eluent of 0˜30% ethyl acetate/petroleum ether) to give tert-butyl 6-[8-(1,3-benzothiazol-2-ylcarbamoyl)-3,4-dihydro-1H-isoquinolin-2-yl]-3-[3-[2-(1-tert-butoxycarbonyl-4-piperidyl)ethoxy]-2-methyl-phenyl]pyridine-2-carboxylate (1.7 g, 2.0 mmol, 69.2% yield, 96.9% purity) was obtained as a yellow solid.

MS (ESI) m/z: 804.5 [M+H]⁺

¹H NMR (400 MHz, DMSO-d₆) δ=12.86 (s, 1H), 8.01 (d, J=7.6 Hz, 1H), 7.78 (d, J=8.0 Hz, 1H), 7.60 (d, J=7.6 Hz, 1H), 7.49-7.41 (m, 3H), 7.39-7.31 (m, 2H), 7.09 (t, J=8.0 Hz, 1H), 6.97-6.89 (m, 2H), 6.57 (d, J=7.6 Hz, 1H), 4.98 (s, 2H), 4.04-3.97 (m, 2H), 3.96-3.83 (m, 4H), 3.07-2.99 (m, 2H), 2.76-2.57 (m, 2H), 1.87 (s, 3H), 1.66 (s, 5H), 1.37 (s, 9H), 1.11-1.03 (m, 2H), 1.00 (s, 9H)

### Step C. Procedure for Preparation of 6-[8-(1,3-benzothiazol-2-ylcarbamoyl)-3,4-dihydro-1H-isoquinolin-2-yl]-3-[2-methyl-3-[2-(4-piperidyl)ethoxy]phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[3-[2-(1-tert-butoxycarbonyl-4-piperidyl)ethoxy]-2-methyl-phenyl]pyridine-2-carboxylate (1.7 g, 2.11 mmol, 1.0 equiv.) and HCl/dioxane (4 M, 17 mL, 32.2 equiv.) was stirred at 25° C. for 2 hours. The mixture was concentrated under reduced pressure to give 6-[8-(1,3-benzothiazol-2-ylcarbamoyl)-3,4-dihydro-1H-isoquinolin-2-yl]-3-[2-methyl-3-[2-(4-piperidyl)ethoxy]phenyl]pyridine-2-carboxylic acid (1.5 g, 1.8 mmol, 88.5% yield, 85.4% purity) as a yellow solid.

MS (ESI) m/z: 648.4 [M+H]⁺

### Step D. Procedure for Preparation of 6-[8-(1,3-benzothiazol-2-ylcarbamoyl)-3,4-dihydro-1H-isoquinolin-2-yl]-3-[3-[2-[1-[[1-[3-(2,6-dioxo-3-piperidyl)-1-methyl-indazol-6-yl]-4-piperidyl]methyl]-4-piperidyl]ethoxy]-2-methyl-phenyl]pyridine-2-carboxylic acid

A solution of 6-[8-(1,3-benzothiazol-2-ylcarbamoyl)-3,4-dihydro-1H-isoquinolin-2-yl]-3-[2-methyl-3-[2-(4-piperidyl)ethoxy]phenyl]pyridine-2-carboxylic acid (150 mg, 231.56 μmol, 1.0 equiv.) and 1-[3-(2,6-dioxo-3-piperidyl)-1-methyl-indazol-6-yl]piperidine-4-carbaldehyde (82.06 mg, 231.56 μmol, 1.0 equiv.) in DCM (5 mL) and IPA (0.5 mL) was stirred at 25° C. for 12 hours. Then NaBH(OAc)₃ (147.23 mg, 694.68 μmol, 3.0 equiv.) was added at 0° C. After the addition, the mixture was stirred at 25° C. for 0.5 hour. The mixture was concentrated under reduced pressure to give a residue, which was diluted with DMF (2 mL) and purified by prep-HPLC to give 6-[8-(1,3-benzothiazol-2-ylcarbamoyl)-3,4-dihydro-1H-isoquinolin-2-yl]-3-[3-[2-[1-[[1-[3-(2,6-dioxo-3-piperidyl)-1-methyl-indazol-6-yl]-4-piperidyl]methyl]-4-piperidyl]ethoxy]-2-methyl-phenyl]pyridine-2-carboxylic acid (40 mg, 37.6 μmol, 16.2% yield, 97.0% purity) as an off-white solid.

MS (ESI) m/z: 986.5 [M+H]⁺

¹H NMR (400 MHz, DMSO-d₆) δ=10.85 (s, 1H), 8.02 (d, J=7.6 Hz, 1H), 7.78 (d, J=8.0 Hz, 1H), 7.62 (d, J=7.2 Hz, 1H), 7.50-7.41 (m, 4H), 7.39-7.31 (m, 2H), 7.11-7.05 (m, 1H), 6.97-6.86 (m, 3H), 6.82-6.76 (m, 1H), 6.64 (d, J=7.6 Hz, 1H), 4.97 (s, 2H), 4.26-4.22 (m, 1H), 3.99 (t, J=6.0 Hz, 2H), 3.91 (t, J=5.6 Hz, 2H), 3.89-3.86 (m, 3H), 3.78-3.74 (m, 2H), 3.57-3.55 (m, 2H), 3.02 (t, J=6.4 Hz, 2H), 2.88-2.83 (m, 2H), 2.74-2.68 (m, 2H), 2.63-2.58 (m, 2H), 2.19-2.15 (m, 2H), 1.95-1.84 (m, 5H), 1.82-1.75 (m, 2H), 1.74-1.64 (m, 5H), 1.55-1.44 (m, 1H), 1.29-1.18 (m, 4H)

### Example 181. Preparation of Compound 266b

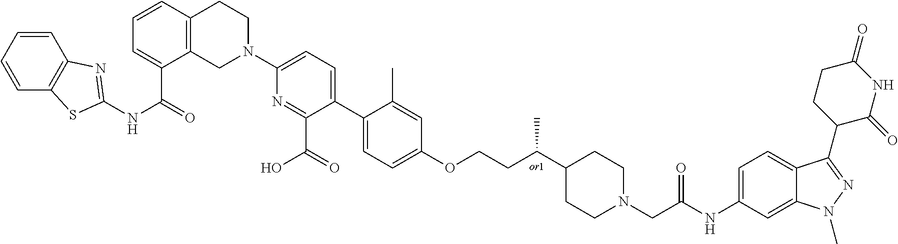 [SMILES: Cc1cc(OCC[C@H](C)C2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[4-[(3S)-3-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylic acid

### [rel-6-(8-(benzo[d]thiazol-2-ylcarbamoyl)-3,4-dihydroisoquinolin-2(1H)-yl)-3-(4-((3S)-3-(1-(2-((3-(2,6-dioxopiperidin-3-yl)-1-methyl-1H-indazol-6-yl)amino)-2-oxoethyl)piperidin-4-yl)butoxy)-2-methylphenyl)picolinic acid]

### Step A. Procedure for Preparation of tert-butyl 4-[3-(4-bromo-3-methyl-phenoxy)-1-methyl-propyl]piperidine-1-carboxylate

To a solution of tert-butyl 4-(3-hydroxy-1-methyl-propyl)piperidine-1-carboxylate (2.2 g, 8.55 mmol, 1.0 equiv.) and 4-bromo-3-methyl-phenol (1.76 g, 9.40 mmol, 1.1 equiv.) in toluene (20 mL) was added 2-(tributyl-λ5-phosphanylidene)acetonitrile (2.48 g, 10.26 mmol, 1.2 equiv.). The mixture was stirred at 120° C. for 12 hours. The mixture was concentrated under reduced pressure to give a residue. The residue was purified by reverse-phase HPLC (with 0.1% HCOOH) to give tert-butyl 4-[3-(4-bromo-3-methyl-phenoxy)-1-methyl-propyl]piperidine-1-carboxylate (2.7 g, 6.1 mmol, 72.1% yield, 97.4% purity) as a brown oil.

MS (ESI) m/z: 448.2 [M+23]⁺

¹H NMR (400 MHz, DMSO-d₆) δ=7.42 (d, J=8.8 Hz, 1H), 6.95 (d, J=2.8 Hz, 1H), 6.71 (dd, J=2.8, 8.8 Hz, 1H), 4.06-3.89 (m, 4H), 2.72-2.54 (m, 2H), 2.29 (s, 3H), 1.84-1.74 (m, 1H), 1.58-1.45 (m, 4H), 1.38 (s, 10H), 1.15-0.97 (m, 2H), 0.85 (d, J=6.4 Hz, 3H)

### Step B. Procedure for Preparation of tert-butyl 4-[(1S)-3-(4-bromo-3-methyl-phenoxy)-1-methyl-propyl]piperidine-1-carboxylate

The compound tert-butyl 4-[3-(4-bromo-3-methyl-phenoxy)-1-methyl-propyl]piperidine-1-carboxylate (1.4 g, 3.28 mmol, 1.0 equiv.) was purified by prep-HPLC to give tert-butyl 4-[(1R)-3-(4-bromo-3-methyl-phenoxy)-1-methyl-propyl]piperidine-1-carboxylate (430 mg, 1.00 mmol, 30.51% yield, 99.32% purity) as a brown oil and tert-butyl 4-[(1S)-3-(4-bromo-3-methyl-phenoxy)-1-methyl-propyl]piperidine-1-carboxylate (380 mg, 865.7 μmol, 26.3% yield, 97.1% purity) as a brown oil

MS (ESI) m/z: 448.2 [M+23]⁺

### Step C. Procedure for Preparation of 4-[(1S)-3-(4-bromo-3-methyl-phenoxy)-1-methyl-propyl]piperidine

A mixture of tert-butyl 4-[(1S)-3-(4-bromo-3-methyl-phenoxy)-1-methyl-propyl]piperidine-1-carboxylate (380 mg, 891.21 μmol, 1.0 equiv.) and HCl/EtOAc (4 M, 4.5 mL, 20.2 equiv.) was stirred at 25° C. for 1 hour. The reaction mixture was concentrated under reduced pressure to give 4-[(1S)-3-(4-bromo-3-methyl-phenoxy)-1-methyl-propyl]piperidine (320 mg, crude) as a yellow solid.

MS (ESI) m/z: 326.0 [M+H]⁺(⁸⁰Br).

### Step D. Procedure for Preparation of ethyl 2-[4-[(1S)-3-(4-bromo-3-methyl-phenoxy)-1-methyl-propyl]-1-piperidyl]acetate

To a solution of 4-[(1S)-3-(4-bromo-3-methyl-phenoxy)-1-methyl-propyl]piperidine (320 mg, 882.19 μmol, 1.0 equiv., HCl) and ethyl 2-bromoacetate (132.59 mg, 793.97 μmol, 87.81 μL, 0.9 equiv.) in CH₃CN (3.5 mL) was added K₂CO₃ (365.77 mg, 2.65 mmol, 3.0 equiv.). The mixture was stirred at 60° C. for 2.5 hours. The reaction mixture was filtered and concentrated under reduced pressure to give an oil. The oil was purified by column chromatography (SiO₂, petroleum ether/ethyl acetate 4/1 to 4/1) to give ethyl 2-[4-[(1S)-3-(4-bromo-3-methyl-phenoxy)-1-methyl-propyl]-1-piperidyl]acetate (228 mg, 519.5 μmol, 58.9% yield, 93.9% purity) as a colorless oil.

MS (ESI) m/z: 412.2 [M+H]⁺.

¹H NMR (400 MHz, DMSO-d₆) δ=7.42 (d, J=8.8 Hz, 1H), 6.95 (d, J=2.4 Hz, 1H), 6.71 (dd, J=2.4, 8.8 Hz, 1H), 4.07 (q, J=7.2 Hz, 2H), 4.02-3.89 (m, 2H), 3.14 (s, 2H), 2.84 (d, J=10.0 Hz, 2H), 2.29 (s, 3H), 2.08 (t, J=11.2 Hz, 2H), 1.85-1.75 (m, 1H), 1.58-1.43 (m, 4H), 1.32-1.21 (m, 2H), 1.21-1.15 (m, 4H), 0.86 (d, J=6.4 Hz, 3H)

### Step E. Procedure for Preparation of tert-butyl 6-[8-(1,3-benzothiazol-2-ylcarbamoyl)-3,4-dihydro-1H-isoquinolin-2-yl]-3-[4-[(3S)-3-[1-(2-ethoxy-2-oxo-ethyl)-4-piperidyl]butoxy]-2-methyl-phenyl]pyridine-2-carboxylate

Ethyl 2-[4-[(1S)-3-(4-bromo-3-methyl-phenoxy)-1-methyl-propyl]-1-piperidyl]acetate (150 mg, 363.76 μmol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (222.82 mg, 363.76 μmol, 1.0 equiv.), KF (63.40 mg, 1.09 mmol, 25.56 μL, 3 equiv.), and Ad₂nBuP Pd G₃ (26.49 mg, 36.38 μmol, 0.1 equiv.) were taken up into a microwave tube in dioxane (2 mL) and H₂O (0.2 mL). The sealed tube was heated at 100° C. for 1 hour under microwave. The reaction mixture was concentrated under reduced pressure to give a residue. The residue was purified by column chromatography (SiO₂, petroleum ether/ethyl acetate=3/1 to 1/1) to give tert-butyl 6-[8-(1,3-benzothiazol-2-ylcarbamoyl)-3,4-dihydro-1H-isoquinolin-2-yl]-3-[4-[(3S)-3-[1-(2-ethoxy-2-oxo-ethyl)-4-piperidyl]butoxy]-2-methyl-phenyl]pyridine-2-carboxylate (160 mg, 180.6 μmol, 49.6% yield, 92.4% purity) as a yellow oil.

MS (ESI) m/z: 410.0 [M12+H]⁺

¹H NMR (400 MHz, DMSO-d₆) δ=12.85 (s, 1H), 8.02 (d, J=7.6 Hz, 1H), 7.77 (d, J=8.0 Hz, 1H), 7.60 (d, J=7.6 Hz, 1H), 7.49-7.41 (m, 3H), 7.39-7.32 (m, 2H), 6.92 (d, J=8.8 Hz, 1H), 6.85 (d, J=8.4 Hz, 1H), 6.82 (d, J=2.4 Hz, 1H), 6.72 (dd, J=2.4, 8.4 Hz, 1H), 4.96 (s, 2H), 4.07-3.92 (m, 5H), 3.86 (t, J=6.0 Hz, 2H), 3.14 (s, 2H), 3.03 (t, J=6.0 Hz, 2H), 2.84 (d, J=9.2 Hz, 2H), 2.08 (t, J=11.2 Hz, 2H), 1.99 (s, 3H), 1.84-1.75 (m, 1H), 1.51 (d, J=12.4 Hz, 4H), 1.31-1.22 (m, 3H), 1.19-1.17 (m, 3H), 1.03 (s, 9H), 0.86 (d, J=6.8 Hz, 3H)

### Step F. Procedure for Preparation of 2-[4-[(1S)-3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]-1-methyl-propyl]-1-piperidyl]acetic acid

To a solution of tert-butyl 6-[8-(1,3-benzothiazol-2-ylcarbamoyl)-3,4-dihydro-1H-isoquinolin-2-yl]-3-[4-[(3S)-3-[1-(2-ethoxy-2-oxo-ethyl)-4-piperidyl]butoxy]-2-methyl-phenyl]pyridine-2-carboxylate (160 mg, 195.59 μmol, 1.0 equiv.) in THE (1.5 mL) and H₂O (0.5 mL) was added LiOH·H₂O (14.05 mg, 586.77 μmol, 3 equiv.). The mixture was stirred at 25° C. for 16 hours. The reaction mixture was concentrated under reduced pressure to remove THF. The resulting residue was diluted with H₂O (10 mL), treated with HCl (1 M) until pH=2, and filtered to give a filter cake, which was dried to give 2-[4-[(1S)-3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]-1-methyl-propyl]-1-piperidyl]acetic acid (65 mg, 74.4 μmol, 38.0% yield, 90.5% purity) as a yellow solid.

MS (ESI) m/z: 790.5 [M+H]⁺

¹H NMR (400 MHz, DMSO-d₆) δ=12.96-12.76 (m, 1H), 8.02 (d, J=7.2 Hz, 1H), 7.77 (d, J=7.2 Hz, 1H), 7.59 (d, J=6.8 Hz, 1H), 7.49-7.30 (m, 5H), 6.92 (d, J=8.4 Hz, 1H), 6.86 (d, J=8.4 Hz, 1H), 6.82 (d, J=2.0 Hz, 1H), 6.73 (d, J=2.4 Hz, 1H), 4.96 (s, 2H), 4.08-3.91 (m, 2H), 3.86 (t, J=5.6 Hz, 2H), 3.05-3.02 (m, 2H), 3.03 (t, J=6.8 Hz, 2H), 2.62-2.54 (m, 5H), 2.00 (s, 3H), 1.83-1.76 (m, 1H), 1.67-1.60 (m, 2H), 1.58-1.41 (m, 4H), 1.38-1.32 (m, 1H), 1.23 (s, 2H), 1.06-0.99 (m, 9H), 0.87 (d, J=6.4 Hz, 3H)

### Step G. Procedure for Preparation of tert-butyl 6-[8-(1,3-benzothiazol-2-ylcarbamoyl)-3,4-dihydro-1H-isoquinolin-2-yl]-3-[4-[(3S)-3-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylate

To a solution of 2-[4-[(1S)-3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]-1-methyl-propyl]-1-piperidyl]acetic acid (60 mg, 75.95 μmol, 1.0 equiv.) in DMF (1 mL) was added HATU (34.65 mg, 91.14 μmol, 1.2 equiv.) and DIEA (29.45 mg, 227.85 μmol, 39.69 μL, 3.0 equiv.). The mixture was stirred at 25° C. for 16 hours. The mixture was added to H₂O (10 mL), and the resulting mixture was filtered to give a filter cake, which was dried to give tert-butyl 6-[8-(1,3-benzothiazol-2-ylcarbamoyl)-3,4-dihydro-1H-isoquinolin-2-yl]-3-[4-[(3S)-3-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylate (106 mg, 102.9 μmol, 135.4% yield) as an off-white solid.

MS (ESI) m/z: 1030.5 [M+H]⁺

¹H NMR (400 MHz, DMSO-d₆) δ=12.85 (s, 1H), 10.88 (s, 1H), 8.02 (d, J=8.0 Hz, 2H), 7.78 (d, J=8.0 Hz, 1H), 7.70-7.62 (m, 1H), 7.60 (d, J=7.6 Hz, 1H), 7.48-7.41 (m, 3H), 7.36 (dd, J=7.2, 13.6 Hz, 2H), 7.23-7.14 (m, 1H), 6.92 (d, J=8.8 Hz, 1H), 6.86 (d, J=8.4 Hz, 1H), 6.83 (d, J=2.4 Hz, 1H), 6.73 (J=2.8, 8.4 Hz, 1H), 4.96 (s, 2H), 4.33 (dd, J=5.2, 10.0 Hz, 1H), 4.05-3.96 (m, 2H), 3.93 (s, 3H), 3.86 (t, J=5.6 Hz, 2H), 3.03 (t, J=6.0 Hz, 2H), 2.63 (dd, J=4.8, 9.6 Hz, 2H), 2.60-2.54 (m, 4H), 2.24-2.11 (m, 2H), 2.01 (s, 3H), 1.91-1.75 (m, 2H), 1.69-1.43 (m, 6H), 1.23 (s, 2H), 1.03 (s, 9H), 0.90 (d, J=6.4 Hz, 3H)

### Step H. Procedure for Preparation of 6-[8-(1,3-benzothiazol-2-ylcarbamoyl)-3,4-dihydro-1H-isoquinolin-2-yl]-3-[4-[(3S)-3-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylic acid [rel-6-(8-(benzo[d]thiazol-2-ylcarbamoyl)-3,4-dihydroisoquinolin-2(1H)-yl)-3-(4-((3S)-3-(1-(2-((3-(2,6-dioxopiperidin-3-yl)-1-methyl-1H-indazol-6-yl)amino)-2-oxoethyl)piperidin-4-yl)butoxy)-2-methylphenyl)picolinic acid]

A solution of tert-butyl 6-[8-(1,3-benzothiazol-2-ylcarbamoyl)-3,4-dihydro-1H-isoquinolin-2-yl]-3-[4-[(3S)-3-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylate (100 mg, 97.06 μmol, 1.0 equiv.) in DCM (0.5 mL) and TFA (0.5 mL) was stirred at 25° C. for 12 hours. The reaction mixture was concentrated under reduced pressure to remove DCM. The resulting residue was diluted with DMF (1.5 mL) and purified by prep-HPLC to give 6-[8-(1,3-benzothiazol-2-ylcarbamoyl)-3,4-dihydro-1H-isoquinolin-2-yl]-3-[4-[(3S)-3-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylic acid [rel-6-(8-(benzo[d]thiazol-2-ylcarbamoyl)-3,4-dihydroisoquinolin-2(1H)-yl)-3-(4-((3S)-3-(1-(2-((3-(2,6-dioxopiperidin-3-yl)-1-methyl-1H-indazol-6-yl)amino)-2-oxoethyl)piperidin-4-yl)butoxy)-2-methylphenyl)picolinic acid] (22.6 mg, 89.8% purity) as a yellow solid.

MS (ESI) m/z: 974.5 [M+H]⁺

¹H NMR (400 MHz, DMSO-d₆) δ=13.04-12.67 (m, 1H), 10.88 (s, 1H), 9.99-9.75 (m, 1H), 8.03 (d, J=9.2 Hz, 2H), 7.79 (d, J=8.0 Hz, 1H), 7.66-7.59 (m, 2H), 7.49-7.42 (m, 3H), 7.40-7.32 (m, 2H), 7.21 (dd, J=1.2, 8.8 Hz, 1H), 6.93 (dd, J=8.4, 14.0 Hz, 2H), 6.80 (d, J=2.4 Hz, 1H), 6.71 (dd, J=2.8, 8.8 Hz, 1H), 4.97 (s, 2H), 4.32 (dd, J=5.2, 9.6 Hz, 1H), 4.02-3.95 (m, 2H), 3.94-3.88 (m, 5H), 3.21-3.15 (m, 2H), 3.02 (t, J=5.2 Hz, 2H), 3.00-2.92 (m, 2H), 2.64-2.60 (m, 2H), 2.40-2.27 (m, 2H), 2.20-2.13 (m, 2H), 2.03 (s, 3H), 1.89-1.82 (m, 1H), 1.65-1.52 (m, 4H), 1.46-1.35 (m, 2H), 1.23 (s, 1H), 0.91 (d, J=6.4 Hz, 3H)

### Example 182. Preparation of Compound 270a

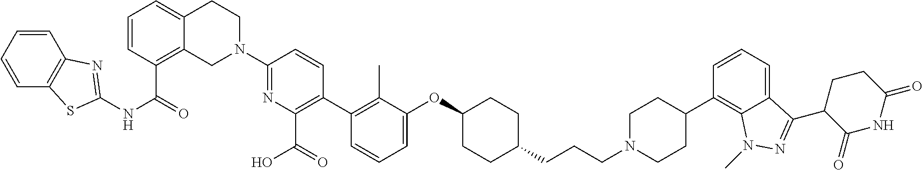 [SMILES: Cc1c(O[C@H]2CC[C@H](CCCN3CCC(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-7-yl)piperidin-1-yl)propyl)cyclohexyl)oxy)-2-methylphenyl)picolinic acid

### Step A. Procedure for Preparation of 3-[4-(3-bromo-2-methyl-phenoxy)cyclohexyl]propan-1-ol

To a solution of ethyl 3-[4-(3-bromo-2-methyl-phenoxy)cyclohexyl]propanoate (600 mg, 1.62 mmol, 1 equiv.) in THE (10 mL) was added LiAlH₄ (55.50 mg, 1.46 mmol, 0.9 equiv.) at 0° C. The mixture was stirred at 0° C. for 1.5 hours. The reaction mixture was quenched by addition H₂O (0.05 mL), 15% NaOH (0.05 mL), and H₂O (15 mL), and then extracted with ethyl acetate (10 mL×3). The combined organic layers were dried over Na₂SO₄, filtered, and concentrated under reduced pressure to give a residue. The residue was used in the next step without purification. The compound 3-[4-(3-bromo-2-methyl-phenoxy)cyclohexyl]propan-1-ol (500 mg, 1.5 mmol, 94.0% yield) was obtained as a white solid.

¹H NMR (400 MHz, DMSO-d₆) δ=7.14-7.02 (m, 3H), 4.23-4.21 (m, 1H), 3.37-3.36 (m, 3H), 3.34-3.35 (m, 1H), 2.26-2.24 (m, 1H), 2.21-2.19 (m, 2H), 2.06-2.04 (m, 2H), 1.76-1.72 (m, 2H), 1.41-1.42 (m, 2H), 1.40-1.39 (m, 2H), 1.38-1.36 (m, 2H), 1.20-1.15 (m, 3H), 1.03-0.99 (m, 2H).

### Step B. Procedure for Preparation of 3-[4-(3-bromo-2-methyl-phenoxy)cyclohexyl]propanal

To a solution of (COCl)₂ (167.23 mg, 1.41 mmol, 101.97 μL, 2 equiv.) in DCM (30 mL) was added dropwise to a solution of DMSO (219.65 mg, 2.81 mmol, 219.65 μL, 4 equiv.) in DCM (30 mL) at −70° C. under N₂ atmosphere. The mixture was stirred at −70° C. for 1 hour. After which time 3-[4-(3-bromo-2-methyl-phenoxy)cyclohexyl]propan-1-ol (230 mg, 702.81 μmol, 1 equiv.) in DCM (30 mL) was added dropwise at −70° C. The solution was stirred for 1 hour at −70° C. Then TEA (426.70 mg, 4.22 mmol, 586.94 μL, 6 equiv.) was added into the solution. The solution was stirred at −70° C. for 1 hour under N₂ atmosphere. The reaction mixture was filtered and concentrated under reduced pressure to give a residue. The residue was purified by flash silica gel chromatography (Eluent of 0˜43% ethyl acetate/petroleum ether) to give 3-[4-(3-bromo-2-methyl-phenoxy)cyclohexyl]propanal (200 mg, 467.3 μmol, 66.5% yield, 76% purity) as a white solid.

### Step C. Procedure for Preparation of tert-butyl 6-[8-(1,3-benzothiazol-2-ylcarbamoyl)-3,4-dihydro-1H-isoquinolin-2-yl]-3-[2-methyl-3-[4-(3-oxopropyl)cyclohexoxy]phenyl]pyridine-2-carboxylate

A mixture of tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (414.34 mg, 676.42 μmol, 1.1 eq), 3-[4-(3-bromo-2-methyl-phenoxy)cyclohexyl]propanal (200 mg, 614.93 μmol, 1 eq), KF (1.5 M, 614.93 uL, 1.5 equiv.), and Ad₂nBuP Pd G₃ (cataCXium® A Pd G₃) (134.35 mg, 184.48 μmol, 0.3 equiv.) in 1,4-dioxane (15 mL) was degassed and purged with N₂ and stirred at 100° C. for 1 hour under microwave. The reaction mixture was filtered and concentrated under reduced pressure to give a residue. The residue was purified by flash silica gel chromatography (Eluent of 0-37% ethyl acetate/petroleum ether). The compound tert-butyl 6-[8-(1,3-benzothiazol-2-ylcarbamoyl)-3,4-dihydro-1H-isoquinolin-2-yl]-3-[2-methyl-3-[4-(3-oxopropyl)cyclohexoxy]phenyl]pyridine-2-carboxylate (300 mg, 410.4 μmol, 66.7% yield) was obtained as a yellow oil.

### Step D. Procedure for Preparation of tert-butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-7-yl]-1-piperidyl]propyl]cyclohexoxy]-2-methyl-phenyl]pyridine-2-carboxylate

A mixture of tert-butyl 6-[8-(1,3-benzothiazol-2-ylcarbamoyl)-3,4-dihydro-1H-isoquinolin-2-yl]-3-[2-methyl-3-[4-(3-oxopropyl)cyclohexoxy]phenyl]pyridine-2-carboxylate (50 mg, 68.41 μmol, 1 equiv.), 3-[1-methyl-7-(4-piperidyl)indazol-3-yl]piperidine-2,6-dione (33.49 mg, 102.61 μmol, 1.5 equiv.) in DCM (1 mL) was degassed and purged with N₂ three times, and then the mixture was stirred at 25° C. for 10 hours under N₂ atmosphere. NaBH(OAc)₃ (29.00 mg, 136.82 μmol, 2 equiv.) was then added to the mixture, which was stirred at 25° C. for 2 hours. The reaction mixture was filtered and concentrated under reduced pressure to give tert-butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-7-yl]-1-piperidyl]propyl]cyclohexoxy]-2-methyl-phenyl]pyridine-2-carboxylate (60 mg, 57.6 μmol, 84.2% yield) as a white solid.

MS (ESI) m/z: 1042.3 [M+H]⁺

### Step E. Procedure for Preparation of 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-7-yl)piperidin-1-yl)propyl)cyclohexyl)oxy)-2-methylphenyl)picolinic acid

To a solution of tert-butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-7-yl]-1-piperidyl]propyl]cyclohexoxy]-2-methyl-phenyl]pyridine-2-carboxylate (50.00 mg, 48.02 μmol, 1 equiv.) in DCM (0.2 mL) was added TFA (5.47 mg, 48.02 μmol, 3.56 μL, 1 equiv.). The mixture was stirred at 25° C. for 0.5 hour. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-7-yl)piperidin-1-yl)propyl)cyclohexyl)oxy)-2-methylphenyl)picolinic acid (21.9 mg, 21.9 μmol, 45.6% yield, 98.4% purity) as a white solid.

MS (ESI) m/z: 493.9 [M12+H]⁺

¹H NMR (400 MHz, DMSO-d₆) δ=10.94-10.82 (m, 1H), 8.17 (s, 1H), 8.03 (d, J=8.4 Hz, 1H), 7.79 (d, J=8.4 Hz, 1H), 7.62 (d, J=7.8 Hz, 1H), 7.49-7.41 (m, 3H), 7.37 (J=7.2 Hz, 3H), 7.07-7.00 (m, 3H), 6.96-6.88 (m, 2H), 6.63-6.61 (m, 1H), 4.97 (s, 2H), 4.35-4.31 (m, 1H), 4.24 (s, 3H), 4.21-4.16 (m, 1H), 3.92-3.89 (m, 2H), 3.03-3.01 (m, 4H), 2.68-2.60 (m, 3H), 2.37-2.34 (m, 4H), 2.22-2.14 (m, 3H), 2.12-2.02 (m, 3H), 1.88 (s, 3H), 1.82-1.78 (m, 2H), 1.52-1.46 (m, 2H), 1.39-1.32 (m, 2H), 1.30-1.19 (m, 4H), 1.10-1.02 (m, 2H).

### Example 183. Preparation of Compound 271a

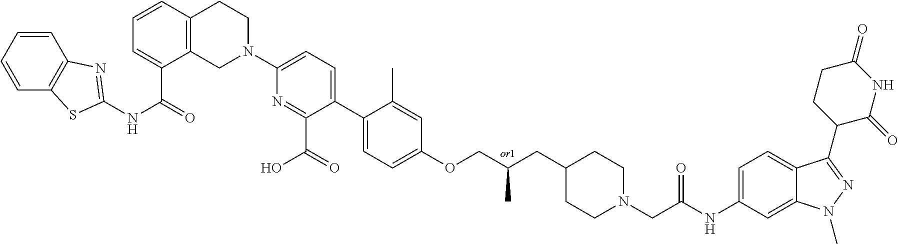 [SMILES: Cc1cc(OC[C@H](C)CC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(4-((2R)-3-(1-(2-((3-(2,6-dioxopiperidin-3-yl)-1-methyl-1H-indazol-6-yl)amino)-2-oxoethyl)piperidin-4-yl)-2-methylpropoxy)-2-methylphenyl)picolinic acid

### [rel-6-(8-(benzo[d]thiazol-2-ylcarbamoyl)-3,4-dihydroisoquinolin-2(1H)-yl)-3-(4-((2R)-3-(1-(2-((3-(2,6-dioxopiperidin-3-yl)-1-methyl-1H-indazol-6-yl)amino)-2-oxoethyl)piperidin-4-yl)-2-methylpropoxy)-2-methylphenyl)picolinic acid]

### Step A. Procedure for Preparation of tert-butyl 4-(3-(4-bromo-3-methylphenoxy)-2-methylpropyl)piperidine-1-carboxylate

A mixture of tert-butyl 4-(3-hydroxy-2-methylpropyl)piperidine-1-carboxylate (2.2 g, 8.55 mmol, 1 equiv.), 4-bromo-3-methyl-phenol (1.92 g, 10.26 mmol, 1.2 equiv.), and 2-(tributyl-λ5-phosphanylidene)acetonitrile (2.48 g, 10.26 mmol, 1.2 equiv.) in toluene (20 mL) was degassed and purged with N₂ and stirred at 120° C. for 4 hours under N₂ atmosphere. The reaction mixture was filtered and concentrated under reduced pressure to give a residue. The residue was purified by prep-HPLC (with HCOOH) to give tert-butyl 4-(3-(4-bromo-3-methylphenoxy)-2-methylpropyl)piperidine-1-carboxylate (2.6 g, 6.0 mmol, 70.8% yield, 99.2% purity) as a yellow oil.

MS (ESI) m/z: 370.7 [M+H]⁺.

¹H NMR (400 MHz, CDCl₃) δ=7.39 (d, J=8.8 Hz, 1H), 6.78 (d, J=2.8 Hz, 1H), 6.60 (dd, J=2.8, 5.6 Hz, 1H), 4.09 (d, J=13.2 Hz, 2H), 3.78-3.66 (m, 2H), 2.73-2.66 (m, 2H), 2.36 (s, 3H), 2.06-2.01 (m, 1H), 1.71-1.64 (m, 2H), 1.46 (s, 9H), 1.44-1.37 (m, 1H), 1.22-1.03 (m, 4H), 1.01 (d, J=6.4 Hz, 3H).

### Step B. Procedure for Preparation of tert-butyl (R)-4-(3-(4-bromo-3-methylphenoxy)-2-methylpropyl)piperidine-1-carboxylate

tert-Butyl 4-(3-(4-bromo-3-methylphenoxy)-2-methylpropyl)piperidine-1-carboxylate obtained from the preceding step was resolved by SFC to give tert-butyl (S)-4-(3-(4-bromo-3-methylphenoxy)-2-methylpropyl)piperidine-1-carboxylate (1 g, 2.28 mmol, 40.58% yield, 97.39% purity) as a yellow oil and tert-butyl (R)-4-(3-(4-bromo-3-methylphenoxy)-2-methylpropyl)piperidine-1-carboxylate (0.9 g, 2.1 mmol, 37.2% yield, 99.4% purity) as a yellow oil.

### Step C. Procedure for Preparation of (R)-4-(3-(4-bromo-3-methylphenoxy)-2-methylpropyl)piperidine

To a solution of tert-butyl (R)-4-(3-(4-bromo-3-methylphenoxy)-2-methylpropyl)piperidine-1-carboxylate (900 mg, 2.11 mmol, 1 equiv.) was added HCl/EtOAc (4 M, 527.69 μL, 1 equiv.). The mixture was stirred at 25° C. for 2 hours. The reaction mixture was filtered and concentrated under reduced pressure to give (R)-4-(3-(4-bromo-3-methylphenoxy)-2-methylpropyl)piperidine (700 mg, crude) as a white solid.

MS (ESI) m/z: 326.7 [M+H]⁺.

### Step D. Procedure for Preparation of ethyl (R)-2-(4-(3-(4-bromo-3-methylphenoxy)-2-methylpropyl)piperidin-1-yl)acetate

To a solution of ethyl 2-bromoacetate (153.55 mg, 919.48 μmol, 101.69 μL, 1 equiv.) and (R)-4-(3-(4-bromo-3-methylphenoxy)-2-methylpropyl)piperidine (300.00 mg, 919.48 μmol, 1 equiv.) in CH₃CN (4 mL) was added K₂CO₃ (381.23 mg, 2.76 mmol, 3 equiv.). The mixture was stirred at 40° C. for 1 hour. The reaction mixture was filtered and concentrated under reduced pressure to give ethyl (R)-2-(4-(3-(4-bromo-3-methylphenoxy)-2-methylpropyl)piperidin-1-yl)acetate (300 mg, crude) as a white solid.

MS (ESI) m/z: 412.0 [M+H]⁺.

### Step E. Procedure for Preparation of tert-butyl (R)-6-(8-(benzo[d]thiazol-2-ylcarbamoyl)-3,4-dihydroisoquinolin-2(1H)-yl)-3-(4-(3-(1-(2-ethoxy-2-oxoethyl)piperidin-4-yl)-2-methylpropoxy)-2-methylphenyl)picolinate

A mixture of tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (222.82 mg, 363.76 μmol, 1 equiv.), ethyl (R)-2-(4-(3-(4-bromo-3-methylphenoxy)-2-methylpropyl)piperidin-1-yl)acetate (150.00 mg, 363.76 μmol, 1 equiv.) in dioxane (3 mL) was added KF (1.5 M, 727.52 μL, 3 equiv.) and Ad₂nBuP Pd G₃ (cataCXium® A Pd G₃) (26.49 mg, 36.38 μmol, 0.1 equiv.). After addition, the mixture was degassed and purged with N₂ three times, and then the mixture was stirred at 100° C. for 2 hours under N₂ atmosphere. The reaction mixture was filtered and concentrated under reduced pressure to give a residue. The residue was purified by flash silica gel chromatography (Eluent of 0˜38% ethyl acetate/petroleum ether) to give compound tert-butyl (R)-6-(8-(benzo[d]thiazol-2-ylcarbamoyl)-3,4-dihydroisoquinolin-2(1H)-yl)-3-(4-(3-(1-(2-ethoxy-2-oxoethyl)piperidin-4-yl)-2-methylpropoxy)-2-methylphenyl)picolinate (170 mg, 182.3 μmol, 50.1% yield, 87.7% purity) as a yellow solid.

MS (ESI) m/z: 819.6 [M+H]⁺.

¹H NMR (400 MHz, CDCl₃) δ=7.84 (d, J=7.6 Hz, 1H), 7.56 (t, J=7.2 Hz, 2H), 7.41-7.28 (m, 5H), 7.00-6.84 (m, 2H), 6.77 (d, J=2.4 Hz, 1H), 6.69 (dd, J=2.8, 5.6 Hz, 1H), 5.04 (s, 2H), 4.22 (q, J=7.2 Hz, 2H), 4.13-4.04 (m, 2H), 3.83-3.69 (m, 2H), 3.38 (S, 2H), 3.18-2.95 (m, 4H), 2.13-1.99 (m, 5H), 1.55-1.43 (m, 4H), 1.34-1.24 (m, 5H), 1.19 (s, 9H), 1.15-1.07 (m, 2H), 1.06-0.98 (m, 3H).

### Step F. Procedure for Preparation of (R)-2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)-2-methylpropyl)piperidin-1-yl)acetic acid

To a solution of tert-butyl (R)-6-(8-(benzo[d]thiazol-2-ylcarbamoyl)-3,4-dihydroisoquinolin-2(1H)-yl)-3-(4-(3-(1-(2-ethoxy-2-oxoethyl)piperidin-4-yl)-2-methylpropoxy)-2-methylphenyl)picolinate (170.00 mg, 207.82 μmol, 1 equiv.) in THF (2 mL) was added LiOH·H₂O (26.16 mg, 623.45 μmol, 3 equiv.) and H₂O (0.5 mL). The mixture was stirred at 25° C. for 2 hours. The reaction mixture was concentrated under reduced pressure to remove THF. The aqueous phase was adjusted to pH=4˜5 with 1M HCl. The reaction mixture was filtered. After addition, the filter cake was diluted in ethyl acetate. The combined organic layers were dried over Na₂SO₄, filtered and concentrated under reduced pressure to give a residue compound (R)-2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)-2-methylpropyl)piperidin-1-yl)acetic acid (160 mg, crude) as a yellow solid.

MS (ESI) m/z: 790.4 [M+H]⁺.

### Step G. Procedure for Preparation of tert-butyl 6-(8-(benzo[d]thiazol-2-ylcarbamoyl)-3,4-dihydroisoquinolin-2(1H)-yl)-3-(4-((2R)-3-(1-(2-((3-(2,6-dioxopiperidin-3-yl)-1-methyl-11H-indazol-6-yl)amino)-2-oxoethyl)piperidin-4-yl)-2-methylpropoxy)-2-methylphenyl)picolinate

To a solution of (R)-2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)-2-methylpropyl)piperidin-1-yl)acetic acid (160.00 mg, 202.54 μmol, 1 equiv.), 3-(6-amino-1-methyl-indazol-3-yl)piperidine-2,6-dione (52.31 mg, 202.54 μmol, 1 equiv.) in pyridine (2 mL) was added EDCI (58.24 mg, 303.81 μmol, 1.5 equiv.). The mixture was stirred at 25° C. for 1 hour. The reaction mixture was quenched by the addition of H₂O (2 mL). The reaction mixture was filtered, and the filter cake was washed with water (5 mL). Afterwards, the filter cake was washed with DCM (10 mL). The combined organic layers were dried over Na₂SO₄, filtered, and concentrated under reduced pressure to give tert-butyl 6-(8-(benzo[d]thiazol-2-ylcarbamoyl)-3,4-dihydroisoquinolin-2(1H)-yl)-3-(4-((2R)-3-(1-(2-((3-(2,6-dioxopiperidin-3-yl)-1-methyl-1H-indazol-6-yl)amino)-2-oxoethyl)piperidin-4-yl)-2-methylpropoxy)-2-methylphenyl)picolinate (160 mg, crude) as a yellow solid.

MS (ESI) m/z: 1030.4 [M+H]⁺.

### Step H. Procedure for Preparation of 6-(8-(benzo[d]thiazol-2-ylcarbamoyl)-3,4-dihydroisoquinolin-2(1H)-yl)-3-(4-((2R)-3-(1-(2-((3-(2,6-dioxopiperidin-3-yl)-1-methyl-1H-indazol-6-yl)amino)-2-oxoethyl)piperidin-4-yl)-2-methylpropoxy)-2-methylphenyl)picolinic acid [rel-6-(8-(benzo[d]thiazol-2-ylcarbamoyl)-3,4-dihydroisoquinolin-2(1H)-yl)-3-(4-((2R)-3-(1-(2-((3-(2,6-dioxopiperidin-3-yl)-1-methyl-1H-indazol-6-yl)amino)-2-oxoethyl)piperidin-4-yl)-2-methylpropoxy)-2-methylphenyl)picolinic acid]

To a solution of tert-butyl 6-(8-(benzo[d]thiazol-2-ylcarbamoyl)-3,4-dihydroisoquinolin-2(1H)-yl)-3-(4-((2R)-3-(1-(2-((3-(2,6-dioxopiperidin-3-yl)-1-methyl-1H-indazol-6-yl)amino)-2-oxoethyl)piperidin-4-yl)-2-methylpropoxy)-2-methylphenyl)picolinate (160.00 mg, 155.30 μmol, 1 equiv.) in DCM (1 mL) was added TFA (1.54 g, 13.51 mmol, 1 mL, 86.97 equiv.). The mixture was stirred at 25° C. for 1 hour. The reaction mixture was filtered and concentrated under reduced pressure to give a residue (100 mg, crude) as a brown oil, which was purified by prep-HPLC to give 6-(8-(benzo[d]thiazol-2-ylcarbamoyl)-3,4-dihydroisoquinolin-2(1H)-yl)-3-(4-((2R)-3-(1-(2-((3-(2,6-dioxopiperidin-3-yl)-1-methyl-1H-indazol-6-yl)amino)-2-oxoethyl)piperidin-4-yl)-2-methylpropoxy)-2-methylphenyl)picolinic acid [rel-6-(8-(benzo[d]thiazol-2-ylcarbamoyl)-3,4-dihydroisoquinolin-2(1H)-yl)-3-(4-((2R)-3-(1-(2-((3-(2,6-dioxopiperidin-3-yl)-1-methyl-1H-indazol-6-yl)amino)-2-oxoethyl)piperidin-4-yl)-2-methylpropoxy)-2-methylphenyl)picolinic acid] (74.6 mg, 70.3 μmol, 68.5% yield, 91.8% purity) as an off white solid.

MS (ESI) m/z: 974.4 [M+H]⁺

¹H NMR (400 MHz, DMSO-d₆) δ=13.11-12.52 (m, 1H), 10.89 (s, 1H), 10.49-9.96 (m, 1H), 8.14 (s, 1H), 8.07-7.99 (m, 2H), 7.79 (d, J=8.0 Hz, 1H), 7.64 (dd, J=8.8, 8.0 Hz, 2H), 7.50-7.41 (m, 3H), 7.41-7.32 (m, 2H), 7.18 (d, J=8.8 Hz, 1H), 6.94 (dd, J=8.8, 4.8 Hz, 2H), 6.79 (s, 1H), 6.76-6.65 (m, 1H), 4.97 (s, 2H), 4.33 (dd, J=5.2, 4.4 Hz, 1H), 3.98-3.89 (m, 5H), 3.88-3.65 (m, 4H), 3.02 (t, J=5.2 Hz, 2H), 2.75-2.54 (m, 4H), 2.35-2.33 (m, 1H), 2.23-2.11 (m, 1H), 2.08-1.91 (m, 5H), 1.78 (d, J=11.6 Hz, 2H), 1.62-1.49 (m, 1H), 1.48-1.30 (m, 3H), 1.23-1.13 (m, 2H), 1.04-0.94 (m, 3H).

### Example 184. Preparation of Compound 282a

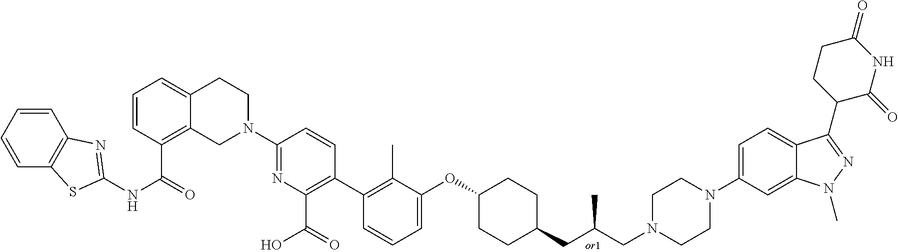 [SMILES: Cc1c(O[C@H]2CC[C@H](C[C@@H](C)CN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1R,4r)-4-((2R)-3-(4-(3-(2,6-dioxopiperidin-3-yl)-1-methyl-1H-indazol-6-yl)piperazin-1-yl)-2-methylpropyl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1R,4r)-4-((2R*)-3-(4-(3-(2,6-dioxopiperidin-3-yl)-1-methyl-1H-indazol-6-yl)piperazin-1-yl)-2-methylpropyl)cyclohexyl)oxy)-2-methylphenyl)picolinic acid]

### Step A. Procedure for Preparation of ((1r,4r)-4-(3-bromo-2-methylphenoxy)cyclohexyl)methanol

To a mixture of methyl (1r,4r)-4-(3-bromo-2-methylphenoxy)cyclohexane-1-carboxylate (40 g, 48.90 mmol, 40% purity, 1 equiv.) in THE (350 mL) was slowly added LiAlH₄ (1.95 g, 51.34 mmol, 1.05 equiv.) at 0° C., and then the mixture was stirred at 25° C. for 2 hours. The mixture was quenched by Na₂SO₄·10 H₂O (3 g). The resulting solution was poured onto ice-water (300 mL) slowly. The pH of the resulting mixture was adjusted to pH 4˜5 with 1 M HCl. The mixture was then extracted with EtOAc (300 mL×3), washed with brine (400 mL), dried over sodium sulfate, filtered, and concentrated under reduced pressure. The residue was purified by flash silica gel chromatography (Eluent of 0˜₁₂% ethyl acetate/petroleum ether) to give ((1r,4r)-4-(3-bromo-2-methylphenoxy)cyclohexyl)methanol (20.05 g, 67.0 mmol, 45.6% yield) as a white solid.

¹H NMR (400 MHz, DMSO-d₆) δ=7.15-7.11 (m, 1H), 7.06 (t, J=8.0 Hz, 1H), 7.03-6.99 (m, 1H), 4.26-4.17 (m, 1H), 2.21 (s, 3H), 2.06 (dd, J=2.4, 11.6 Hz, 2H), 1.98 (s, 1H), 1.78 (d, J=11.6 Hz, 2H), 1.58 (d, J=4.8 Hz, 2H), 1.40-1.34 (m, 5H)

### Step B. Procedure for Preparation of (1r,4r)-4-(3-bromo-2-methylphenoxy)cyclohexane-1-carbaldehyde

To a mixture of (COCl)₂ (8.48 g, 66.84 mmol, 5.85 mL, 2 equiv.) in DCM (80 mL) at −78° C. was added DMSO (10.45 g, 133.69 mmol, 10.45 mL, 4 equiv.). The mixture was stirred for 0.5 hours and was then treated with ((1r,4r)-4-(3-bromo-2-methylphenoxy)cyclohexyl)methanol (10 g, 33.42 mmol, 1 equiv.) in DCM (200 mL) and stirred at −78° C. for 1 hour. Then the mixture was treated with TEA (20.29 g, 200.53 mmol, 27.91 mL, 6 equiv.) and warmed to 25° C. following by stirring for 0.5 hour under N₂ atmosphere. The reaction mixture was diluted with H₂O (150 mL) and extracted with DCM (100 mL×3). The combined organic layers were washed with brine (200 mL), filtered, and concentrated under reduced pressure to give (1r,4r)-4-(3-bromo-2-methylphenoxy)cyclohexane-1-carbaldehyde (33.2 g, crude) as a white solid

MS (ESI) m/z: 296.9 [M+H]⁺.

¹H NMR (400 MHz, DMSO-d₆) δ=9.61 (s, 1H), 7.16-7.13 (m, 1H), 7.10-7.05 (m, 1H), 7.04-7.01 (m, 1H), 4.34-4.27 (m, 1H), 2.39-2.33 (m, 1H), 2.25-2.20 (m, 3H), 2.03-1.93 (m, 4H), 1.60-1.55 (m, 2H), 1.46 (d, J=9.6 Hz, 2H)

### Step C. Procedure for Preparation of (R)-3-((1r,4R)-4-(3-bromo-2-methylphenoxy)cyclohexyl)-2-methylpropan-1-ol

A mixture of NaH (1.13 g, 28.27 mmol, 60% purity, 2.1 equiv.) in THE (100 mL) was purged with N₂ for three times. To the mixture was slowly added ethyl 2-diethoxyphosphorylpropanoate (6.41 g, 26.92 mmol, 5.88 mL, 2 equiv.) at 0° C. The mixture was stirred for 2 hours under N₂ atmosphere, treated with (1r,4r)-4-(3-bromo-2-methylphenoxy)cyclohexane-1-carbaldehyde (4.00 g, 13.46 mmol, 1 equiv.) at 0° C., and warm to 25° C. followed by stirring for 3 hours under N₂ atmosphere. The reaction mixture was quenched by addition saturated NH₄Cl (100 mL) under 0° C. The mixture was extracted with DCM (50 mL×3). The combined organic layers were washed with brine (100 mL×2), dried over Na₂SO₄, filtered, and concentrated under reduced pressure to give a residue. The residue was purified by flash silica gel chromatography (Eluent of 0-5% ethyl acetate/petroleum ether) to give (R)-3-((1r,4R)-4-(3-bromo-2-methylphenoxy)cyclohexyl)-2-methylpropan-1-ol (1.1 g, 2.88 mmol, 21.43% yield) as a yellow solid.

¹H NMR (400 MHz, DMSO-d₆) δ=7.18-7.00 (m, 3H), 6.53-6.46 (m, 1H), 4.34-4.24 (m, 1H), 4.16-4.06 (m, 2H), 2.43-2.37 (m, 1H), 2.22 (s, 3H), 2.10-2.03 (m, 2H), 1.81 (s, 3H), 1.72-1.64 (m, 2H), 1.52-1.42 (m, 2H), 1.38-1.30 (m, 2H), 1.21 (br t, J=7.2 Hz, 3H)

### Step D. Procedure for Preparation of ethyl 3-[4-(3-bromo-2-methyl-phenoxy)cyclohexyl]-2-methyl-propanoate

To a solution of ethyl (E)-3-[4-(3-bromo-2-methyl-phenoxy)cyclohexyl]-2-methyl-prop-2-enoate (1 g, 2.62 mmol, 1 equiv.) in THE (15 mL) was added PtO₂ (119.11 mg, 524.52 μmol, 0.2 equiv.) under H₂ atmosphere (15 psi). The mixture was stirred at 25° C. for 16 hours. The reaction mixture was filtered and concentrated under reduced pressure to give a little cake which was rinsed with EtOH. The filtrate was collected and concentrated it under reduced pressure to give a residue. The residue was purified by column chromatography (SiO₂, petroleum ether/ethyl acetate 0/1 to 80/1) to give ethyl 3-[4-(3-bromo-2-methyl-phenoxy)cyclohexyl]-2-methyl-propanoate (950 mg, 2.48 mmol, 94.50% yield) as a yellow oil.

¹H NMR (400 MHz, CDCl₃) δ=7.14 (d, J=8.0 Hz, 1H), 6.97 (t, J=8.0 Hz, 1H), 6.87-6.76 (m, 1H), 4.20-4.03 (m, 3H), 2.58-2.47 (m, 1H), 2.36-2.20 (m, 3H), 2.18-2.08 (m, 2H), 1.94-1.86 (m, 1H), 1.84-1.76 (m, 1H), 1.69-1.61 (m, 1H), 1.50-1.38 (m, 2H), 1.36-1.24 (m, 5H), 1.15 (d, J=6.8 Hz, 3H), 1.08-0.96 (m, 2H)

### Step E. Procedure for Preparation of 3-[4-(3-bromo-2-methyl-phenoxy)cyclohexyl]-2-methyl-propan-1-ol

To a solution of ethyl 3-[4-(3-bromo-2-methyl-phenoxy)cyclohexyl]-2-methyl-propanoate (950 mg, 2.48 mmol, 1 equiv.) in THE (15 mL) was added LiAlH₄ (94.05 mg, 2.48 mmol, 1 equiv.). The mixture was stirred at 0° C. for 2 hours under N₂ atmosphere The reaction mixture was quenched by addition H₂O (0.1 mL), 15% NaOH (0.1 mL), and H₂O (0.3 mL), The combined mixture was mixed with Na₂SO₄, then filtered and concentrated under reduced pressure to give a residue. The residue was purified by prep-HPLC to give 3-[4-(3-bromo-2-methyl-phenoxy)cyclohexyl]-2-methyl-propan-1-ol (280 mg, 814.4 μmol, 32.8% yield, 99.2% purity) as a yellow oil.

MS (ESI) m/z: 342.2 [M+H]⁺.

¹H NMR (400 MHz, CDCl₃) δ=7.13 (d, J=8.0 Hz, 1H), 6.96 (t, J=8.0 Hz, 1H), 6.80 (d, J=8.0 Hz, 1H), 4.10-4.04 (m, 1H), 3.55-3.48 (m, 1H), 3.46-3.39 (m, 1H), 2.29 (s, 3H), 2.16-2.09 (m, 2H), 1.90-1.77 (m, 3H), 1.77-1.68 (m, 1H), 1.48-1.36 (m, 4H), 1.08-0.96 (m, 3H), 0.92 (d, J=6.4 Hz, 3H)

### Step F. Procedure for Preparation of (2R)-3-[4-(3-bromo-2-methyl-phenoxy)cyclohexyl]-2-methyl-propan-1-ol

The 3-[4-(3-bromo-2-methyl-phenoxy)cyclohexyl]-2-methyl-propan-1-ol residue was separated by prep-HPLC to give (R)-3-((1r,4R)-4-(3-bromo-2-methylphenoxy)cyclohexyl)-2-methylpropan-1-ol (100 mg, 293.01 μmol, 35.71% yield) and (2S)-3-[4-(3-bromo-2-methyl-phenoxy)cyclohexyl]-2-methyl-propan-1-ol (100 mg, 293.01 μmol, 35.71% yield) as a yellow oil.

### Step G. Procedure for Preparation of (R)-3-((1r,4R)-4-(3-bromo-2-methylphenoxy)cyclohexyl)-2-methylpropanal

A solution of oxalyl dichloride (74.38 mg, 586.03 μmol, 51.30 μL, 2 equiv.) in DCM (5 mL) was added dropwise to a solution of DMSO (91.57 mg, 1.17 mmol, 91.57 μL, 4 equiv.) in DCM (2 mL) at −70° C. under N₂ atmosphere. The mixture was stirred at −70° C. for 30 minutes. After which time (2R)-3-[4-(3-bromo-2-methyl-phenoxy)cyclohexyl]-2-methyl-propan-1-ol (100 mg, 293.01 μmol, 1 equiv.) in DCM (2 mL) was added dropwise at −70° C. The solution was stirred for 30 minutes at −70° C. Then TEA (177.90 mg, 1.76 mmol, 244.70 μL, 6 equiv.) was added into the solution. The solution was stirred at −70° C. for 2 hours under N₂ atmosphere. The reaction was diluted with water (60 mL) and extracted with DCM (40 mL×2). The combined organic layers were washed with brine (60 mL), dried over Na₂SO₄, filtered, and concentrated under reduced pressure to give (R)-3-((1r,4R)-4-(3-bromo-2-methylphenoxy)cyclohexyl)-2-methylpropanal (90 mg, 265.28 μmol, 90.53% yield) as a yellow oil.

### Step H. Procedure for Preparation of 3-[6-[4-[(2R)-3-[4-(3-bromo-2-methyl-phenoxy)cyclohexyl]-2-methyl-propyl]piperazin-1-yl]-1-methyl-indazol-3-yl]piperidine-2,6-dione

To a solution of 3-(1-methyl-6-piperazin-1-yl-indazol-3-yl)piperidine-2,6-dione (84.92 mg, 259.38 μmol, 1.1 equiv.) in DCM (1 mL) was added NaBH(OAc)₃ (149.93 mg, 707.41 μmol, 3 equiv.), AcOH (28.32 mg, 471.61 μmol, 26.97 μL, 2 equiv.), and (2R)-3-[4-(3-bromo-2-methyl-phenoxy)cyclohexyl]-2-methyl-propanal (80 mg, 235.80 μmol, 1 equiv.) at 0° C. The mixture was stirred at 25° C. for 1 hour. The reaction was diluted with water (40 mL) and extracted with DCM (25 mL×2). The combined organic layers were washed with brine (50 mL), dried over Na₂SO₄, filtered, and concentrated under reduced pressure to give a residue. The residue was purified by prep-TLC (SiO₂, DCM:MeOH=20:1) to give 3-[6-[4-[(2R)-3-[4-(3-bromo-2-methyl-phenoxy)cyclohexyl]-2-methyl-propyl]piperazin-1-yl]-1-methyl-indazol-3-yl]piperidine-2,6-dione (65 mg, 99.90 μmol, 42.37% yield) as a white solid.

MS (ESI) m/z: 650.4 [M+H]⁺.

### Step I. Procedure for preparation of tert-butyl 6-[8-(1,3-benzothiazol-2-ylcarbamoyl)-3,4-dihydro-1H-isoquinolin-2-yl]-3-[3-[4-[(2R)-3-[4-[3-(2,6-dioxo-3-piperidyl)-1-methyl-indazol-6-yl]piperazin-1-yl]-2-methyl-propyl]cyclohexoxy]-2-methyl-phenyl]pyridine-2-carboxylate

To a solution of tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (60 mg, 97.95 μmol, 1 equiv.) in dioxane (2 mL) and H₂O (0.5 mL) was added [2-(2-aminophenyl)phenyl]palladium(1⁺); bis(1-adamantyl)-butyl-phosphane; methanesulfonate (7.13 mg, 9.80 μmol, 0.1 equiv.), 3-[6-[4-[(2R)-3-[4-(3-bromo-2-methyl-phenoxy)cyclohexyl]-2-methyl-propyl]piperazin-1-yl]-1-methyl-indazol-3-yl]piperidine-2,6-dione (63.73 mg, 97.95 μmol, 1 equiv.), and KF (17.07 mg, 293.86 μmol, 6.88 μL, 3 equiv.). The mixture was stirred at 100° C. for 1 hour. The reaction was diluted with water (20 mL) and extracted with DCM (10 mL×2). The combined organic layers were washed with brine (30 mL), dried over Na₂SO₄, filtered, and concentrated under reduced pressure to give as a residue. The residue was purified by column chromatography (SiO₂, dichloromethane: methanol=50/1 to 20/1) to give tert-butyl 6-[8-(1,3-benzothiazol-2-ylcarbamoyl)-3,4-dihydro-1H-isoquinolin-2-yl]-3-[3-[4-[(2R)-3-[4-[3-(2,6-dioxo-3-piperidyl)-1-methyl-indazol-6-yl]piperazin-1-yl]-2-methyl-propyl]cyclohexoxy]-2-methyl-phenyl]pyridine-2-carboxylate (50 mg, 47.33 μmol, 48.32% yield) as a yellow solid.

MS (ESI) m/z: 1056.4 [M+H]⁺.

### Step J. Procedure for Preparation of 6-(8-(benzo[d]thiazol-2-ylcarbamoyl)-3,4-dihydroisoquinolin-2(1H)-yl)-3-(3-(((1R,4r)-4-((2R)-3-(4-(3-(2,6-dioxopiperidin-3-yl)-1-methyl-1H-indazol-6-yl)piperazin-1-yl)-2-methylpropyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1R,4r)-4-((2R*)-3-(4-(3-(2,6-dioxopiperidin-3-yl)-1-methyl-1H-indazol-6-yl)piperazin-1-yl)-2-methylpropyl)cyclohexyl)oxy)-2-methylphenyl)picolinic acid]

To a solution of tert-butyl 6-[8-(1,3-benzothiazol-2-ylcarbamoyl)-3,4-dihydro-1H-isoquinolin-2-yl]-3-[3-[4-[(2R)-3-[4-[3-(2,6-dioxo-3-piperidyl)-1-methyl-indazol-6-yl]piperazin-1-yl]-2-methyl-propyl]cyclohexoxy]-2-methyl-phenyl]pyridine-2-carboxylate (50 mg, 47.33 μmol, 1 equiv.) in DCM (0.5 mL) was added TFA (770.00 mg, 6.75 mmol, 0.5 mL, 142.67 equiv.). The mixture was stirred at 40° C. for 1 hour. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1R,4r)-4-((2R)-3-(4-(3-(2,6-dioxopiperidin-3-yl)-1-methyl-1H-indazol-6-yl)piperazin-1-yl)-2-methylpropyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1R,4r)-4-((2R*)-3-(4-(3-(2,6-dioxopiperidin-3-yl)-1-methyl-1H-indazol-6-yl)piperazin-1-yl)-2-methylpropyl)cyclohexyl)oxy)-2-methylphenyl)picolinic acid] (15.2 mg, 14.9 μmol, 31.6% yield, 98.6% purity) as a yellow solid.

MS (ESI) m/z: 1000.4 [M+H]⁺.

¹H NMR (400 MHz, DMSO-d₆) δ=12.91-12.80 (m, 1H), 10.85 (s, 1H), 8.04 (d, J=8.0 Hz, 1H), 7.80 (d, J=8.0 Hz, 1H), 7.63 (d, J=7.6 Hz, 1H), 7.53-7.49 (m, 1H), 7.49-7.43 (m, 3H), 7.41-7.33 (m, 2H), 7.11-7.05 (m, 1H), 6.99-6.86 (m, 4H), 6.62 (d, J=7.6 Hz, 1H), 4.98 (s, 2H), 4.29-4.18 (m, 2H), 3.92 (s, 2H), 3.90 (s, 3H), 3.23 (d, J=5.2 Hz, 4H), 3.03 (t, J=5.2 Hz, 2H), 2.66-2.55 (m, 8H), 2.35-2.30 (m, 1H), 2.18 (d, J=6.0 Hz, 1H), 2.11-2.06 (m, 2H), 1.87 (s, 3H), 1.83-1.73 (m, 2H), 1.48-1.27 (m, 5H), 1.10 (d, J=11.2 Hz, 1H), 1.05-0.97 (m, 2H), 0.94-0.86 (m, 3H)

### Example 185. Preparation of Compound 282b

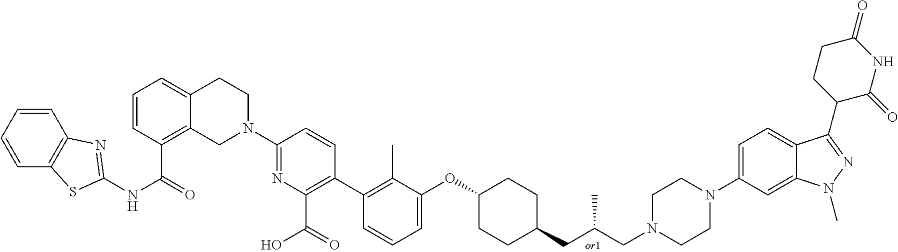 [SMILES: Cc1c(O[C@H]2CC[C@H](C[C@H](C)CN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1S,4r)-4-((2S)-3-(4-(3-(2,6-dioxopiperidin-3-yl)-1-methyl-1H-indazol-6-yl)piperazin-1-yl)-2-methylpropyl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1S,4r)-4-((2S*)-3-(4-(3-(2,6-dioxopiperidin-3-yl)-1-methyl-1H-indazol-6-yl)piperazin-1-yl)-2-methylpropyl)cyclohexyl)oxy)-2-methylphenyl)picolinic acid]

### Step A. Procedure for Preparation of (S)-3-((1r,4S)-4-(3-bromo-2-methylphenoxy)cyclohexyl)-2-methylpropanal

To a solution of oxalyl dichloride (74.38 mg, 586.03 μmol, 51.30 μL, 2 equiv.) in DCM (5 mL) was added a solution of DMSO (91.58 mg, 1.17 mmol, 91.58 μL, 4 equiv.) in DCM (2 mL) dropwise at −70° C. under N₂ atmosphere. The mixture was stirred at −70° C. for 1 hour. After which time (2S)-3-[4-(3-bromo-2-methyl-phenoxy)cyclohexyl]-2-methyl-propan-1-ol (100 mg, 293.01 μmol, 1 equiv.) in DCM (2 mL) was added dropwise at −70° C. The solution was stirred for 1 hour at −70° C. Then TEA (177.90 mg, 1.76 mmol, 244.70 μL, 6 equiv.) was added into the solution. The solution was stirred at −70° C. for 0.5 hour under N₂ atmosphere, after which water (30 mL) was added, and the reaction was extracted with EtOAc (10 mL×2), dried over anhydrous sodium sulfate, filtered, and concentrated under vacuum to give (S)-3-((1r,4S)-4-(3-bromo-2-methylphenoxy)cyclohexyl)-2-methylpropanal (80 mg, crude) as a yellow oil.

### Step B. Procedure for Preparation of 3-(6-(4-((S)-3-((1r,4S)-4-(3-bromo-2-methylphenoxy)cyclohexyl)-2-methylpropyl)piperazin-1-yl)-1-methyl-1H-indazol-3-yl)piperidine-2,6-dione

To a solution of 3-(1-methyl-6-(piperazin-1-yl)-1H-indazol-3-yl)piperidine-2,6-dione (90.08 mg, 247.59 μmol, 1.2 equiv., HCl) in DCM (2 mL) was added NaBH(OAc)₃ (131.19 mg, 618.98 μmol, 3 equiv.). (S)-3-((1r,4S)-4-(3-bromo-2-methylphenoxy)cyclohexyl)-2-methylpropanal (70 mg, 206.33 μmol, 1 equiv.) was then added into the mixture, which was stirred at 0° C. for 1 hour. Then the mixture was stirred at 25° C. for 15 hours. The mixture was filtered and concentrated under vacuum. The residue was purified by column chromatography (SiO₂, petroleum ether/ethyl acetate=4/1 to DCM:MeOH=10:1) to give 3-(6-(4-((S)-3-((1r,4S)-4-(3-bromo-2-methylphenoxy)cyclohexyl)-2-methylpropyl)piperazin-1-yl)-1-methyl-1H-indazol-3-yl)piperidine-2,6-dione (70 mg, 107.59 μmol, 52.14% yield) as a yellow solid.

MS (ESI) m/z: 650.1 [M+H]⁺

### Step C. Procedure for Preparation of tert-butyl 6-(8-(benzo[d]thiazol-2-ylcarbamoyl)-3,4-dihydroisoquinolin-2(1H)-yl)-3-(3-(((1S,4r)-4-((2S)-3-(4-(3-(2,6-dioxopiperidin-3-yl)-1-methyl-1H-indazol-6-yl)piperazin-1-yl)-2-methylpropyl)cyclohexyl)oxy)-2-methylphenyl)picolinate

A mixture of 3-(6-(4-((S)-3-((1r,4S)-4-(3-bromo-2-methylphenoxy)cyclohexyl)-2-methylpropyl)piperazin-1-yl)-1-methyl-1H-indazol-3-yl)piperidine-2,6-dione (70 mg, 107.59 μmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (79.08 mg, 129.10 μmol, 1.2 equiv.), H₂O (0.1 mL), KF (18.75 mg, 322.76 μmol, 7.56 μL, 3 equiv.), and [2-(2-aminophenyl)phenyl]palladium(1⁺); bis(1-adamantyl)-butyl-phosphane; methanesulfonate (7.84 mg, 10.76 μmol, 0.1 equiv.) in dioxane (1 mL) was degassed and purged with N₂ and stirred at 100° C. for 1 hour under N₂ atmosphere. The reaction mixture was concentrated under reduced pressure to give a residue. The residue was purified by flash silica gel chromatography (Eluent of 0-25% ethyl acetate/petroleum ether) to give tert-butyl 6-(8-(benzo[d]thiazol-2-ylcarbamoyl)-3,4-dihydroisoquinolin-2(1H)-yl)-3-(3-(((1S,4r)-4-((2S)-3-(4-(3-(2,6-dioxopiperidin-3-yl)-1-methyl-1H-indazol-6-yl)piperazin-1-yl)-2-methylpropyl)cyclohexyl)oxy)-2-methylphenyl)picolinate (60 mg, 56.80 μmol, 52.80% yield) as a yellow solid.

MS (ESI) m/z: 1056.7 [M+H]⁺

### Step D. Procedure for Preparation of 6-(8-(benzo[d]thiazol-2-ylcarbamoyl)-3,4-dihydroisoquinolin-2(1H)-yl)-3-(3-(((1S,4r)-4-((2S)-3-(4-(3-(2,6-dioxopiperidin-3-yl)-1-methyl-1H-indazol-6-yl)piperazin-1-yl)-2-methylpropyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1S,4r)-4-((2S*)-3-(4-(3-(2,6-dioxopiperidin-3-yl)-1-methyl-1H-indazol-6-yl)piperazin-1-yl)-2-methylpropyl)cyclohexyl)oxy)-2-methylphenyl)picolinic acid]

To a solution of tert-butyl 6-(8-(benzo[d]thiazol-2-ylcarbamoyl)-3,4-dihydroisoquinolin-2(1H)-yl)-3-(3-(((1S,4r)-4-((2S)-3-(4-(3-(2,6-dioxopiperidin-3-yl)-1-methyl-1H-indazol-6-yl)piperazin-1-yl)-2-methylpropyl)cyclohexyl)oxy)-2-methylphenyl)picolinate (60.00 mg, 56.80 μmol, 1 equiv.) in DCM (1 mL) was added TFA (1.54 g, 13.51 mmol, 1.00 mL, 237.78 equiv.). The mixture was stirred at 25° C. for 16 hours. The reaction mixture was concentrated under reduced pressure to remove solvent. The residue was purified by prep-HPLC to give 6-(8-(benzo[d]thiazol-2-ylcarbamoyl)-3,4-dihydroisoquinolin-2(1H)-yl)-3-(3-(((1S,4r)-4-((2S)-3-(4-(3-(2,6-dioxopiperidin-3-yl)-1-methyl-1H-indazol-6-yl)piperazin-1-yl)-2-methylpropyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1S,4r)-4-((2S*)-3-(4-(3-(2,6-dioxopiperidin-3-yl)-1-methyl-1H-indazol-6-yl)piperazin-1-yl)-2-methylpropyl)cyclohexyl)oxy)-2-methylphenyl)picolinic acid] (19.87 mg, 18.78 μmol, 33.07% yield, 98.90% purity, FA) was obtained as a yellow solid.

MS (ESI) m/z: 1000.3 [M+H]⁺

¹H NMR (400 MHz, DMSO) δ=13.04-12.72 (m, 1H), 12.69-12.33 (m, 1H), 10.86 (s, 1H), 8.14 (s, 1H), 8.04 (d, J=8.0 Hz, 1H), 7.79 (d, J=8.0 Hz, 1H), 7.63 (d, J=6.8 Hz, 1H), 7.51-7.44 (m, 4H), 7.40-7.34 (m, 2H), 7.10-7.04 (m, 1H), 6.99-6.90 (m, 3H), 6.85 (s, 1H), 6.61 (d, J=7.6 Hz, 1H), 4.98 (s, 2H), 4.29-4.17 (m, 2H), 3.96-3.90 (m, 2H), 3.89 (s, 3H), 3.23 (s, 4H), 3.03 (t, J=5.6 Hz, 2H), 2.65-2.59 (m, 2H), 2.55-2.53 (m, 2H), 2.36-2.24 (m, 2H), 2.21-2.05 (m, 6H), 1.87 (s, 3H), 1.86-1.70 (m, 4H), 1.44-1.32 (m, 4H), 1.04-0.96 (m, 2H), 0.88 (d, J=6.4 Hz, 3H)

### Example 186. Preparation of Compound 285

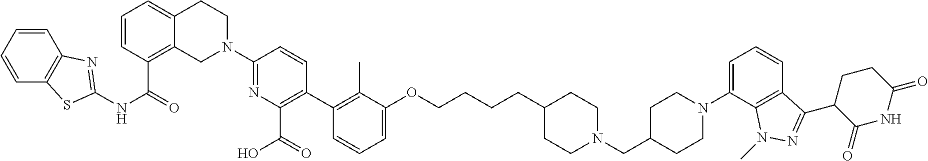 [SMILES: Cc1c(OCCCCC2CCN(CC3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[4-[1-[[1-[3-(2,6-dioxo-3-piperidyl)-1-methyl-indazol-7-yl]-4-piperidyl]methyl]-4-piperidyl]butoxy]-2-methyl-phenyl]pyridine-2-carboxylic acid

### Step A. Procedure for Preparation of 6-[8-(1,3-benzothiazol-2-ylcarbamoyl)-3,4-dihydro-1H-isoquinolin-2-yl]-3-[3-[4-[1-[[1-[3-(2,6-dioxo-3-piperidyl)-1-methyl-indazol-7-yl]-4-piperidyl]methyl]-4-piperidyl]butoxy]-2-methyl-phenyl]pyridine-2-carboxylic acid

To a solution of 6-[8-(1,3-benzothiazol-2-ylcarbamoyl)-3,4-dihydro-1H-isoquinolin-2-yl]-3-[2-methyl-3-[4-(4-piperidyl)butoxy]phenyl]pyridine-2-carboxylic acid (158.28 mg, 234.20 μmol, 1 equiv.) in DCM (1 mL) and IPA (1 mL) was added 1-[3-(2,6-dioxo-3-piperidyl)-1-methyl-indazol-7-yl]piperidine-4-carbaldehyde (83 mg, 234.20 μmol, 1 equiv.) at 25° C. for 11 hours. The mixture was stirred at this temperature, and then NaBH(OAc)₃ (148.91 mg, 702.59 μmol, 3 equiv.) was added at 0° C. The resulting mixture was stirred at 25° C. for 1 hour. The mixture was concentrated to give a residue. The residue was purified by reverse-phase HPLC to give 6-[8-(1,3-benzothiazol-2-ylcarbamoyl)-3,4-dihydro-1H-isoquinolin-2-yl]-3-[3-[4-[1-[[1-[3-(2,6-dioxo-3-piperidyl)-1-methyl-indazol-7-yl]-4-piperidyl]methyl]-4-piperidyl]butoxy]-2-methyl-phenyl]pyridine-2-carboxylic acid (27.7 mg, 26.2 μmol, 11.2% yield, 96.0% purity) as an off-white solid.

MS (ESI) m/z: 508.1 [M/2+H]⁺.

¹H NMR (400 MHz, DMSO-d₆) δ=10.87 (s, 1H), 8.18 (s, 1H), 8.01 (d, J=7.6 Hz, 1H), 7.78 (d, J=8.4 Hz, 1H), 7.62 (d, J=6.8 Hz, 1H), 7.49-7.29 (m, 6H), 7.10-7.04 (m, 1H), 7.02-6.96 (m, 2H), 6.94-6.83 (m, 2H), 6.64 (d, J=7.6 Hz, 1H), 4.96 (s, 2H), 4.33 (d, J=5.2, 9.6 Hz, 1H), 4.23 (s, 3H), 3.97 (t, J=6.0 Hz, 2H), 3.90 (t, J=5.6 Hz, 2H), 3.21-3.19 (m, 2H), 3.00 (t, J=5.6 Hz, 2H), 2.87 (dd, J=2.0, 8.4 Hz, 2H), 2.68-2.61 (m, 4H), 2.33 (s, 1H), 2.23 (d, J=3.6 Hz, 2H), 2.20-2.13 (m, 1H), 1.96-1.88 (m, 5H), 1.84 (d, J=11.6 Hz, 2H), 1.76-1.66 (m, 3H), 1.65-1.59 (m, 2H), 1.48-1.40 (m, 2H), 1.38-1.21 (m, 5H), 1.19-1.07 (m, 2H).

### Example 187. Preparation of Compound 106

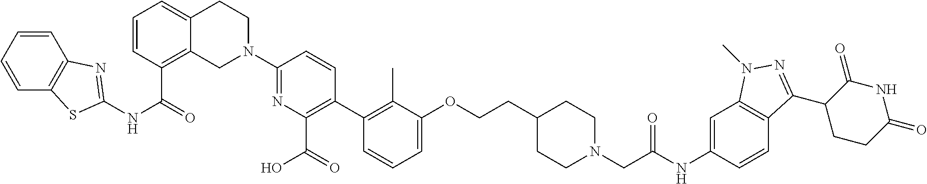 [SMILES: Cc1c(OCCC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[2-[1-[2-[[3-(2,6-dioxo-3-piperidyl)-1-methyl-indazol-6-yl]amino]-2-oxo-ethyl]-4-piperidyl]ethoxy]-2-methyl-phenyl]pyridine-2-carboxylic acid

### Step A. Procedure for Preparation of 3-(2,6-dibenzyloxy-3-pyridyl)-1-methyl-6-nitro-indazole

To a solution of (2,6-dibenzyloxy-3-pyridyl)boronic acid (4.5 g, 13.43 mmol, 1 equiv.) and 3-iodo-1-methyl-6-nitro-indazole (4.48 g, 14.77 mmol, 1.1 equiv.) in dioxane (45 mL) and H₂O (4.5 mL) was added Na₂CO₃ (4.27 g, 40.28 mmol, 3 equiv.) and Pd(dppf)Cl₂ (982.42 mg, 1.34 mmol, 0.1 equiv.). The mixture was stirred at 100° C. for 2 hours. The reaction mixture was filtered and concentrated under reduced pressure to give a residue. The residue was purified by column chromatography (SiO₂, petroleum ether/ethyl acetate=1/0 to 5/1). The compound 3-(2,6-dibenzyloxy-3-pyridyl)-1-methyl-6-nitro-indazole (3.8 g, 8.1 mmol, 60.6% yield) was obtained as a yellow solid.

MS (ESI) m/z: 467.1 [M+H]⁺.

### Step B. Procedure for Preparation of 3-(2,6-dibenzyloxy-3-pyridyl)-1-methyl-indazol-6-amine

A mixture of 3-(2,6-dibenzyloxy-3-pyridyl)-1-methyl-6-nitro-indazole (3.8 g, 8.15 mmol, 1 equiv.), Pd/C (900 mg, 10% purity), Pd(OH)₂ (900 mg, 20% purity) in EtOH (40 mL) and THE (40 mL) was degassed and purged with H₂ and stirred at 50° C. for 12 hours under H₂ atmosphere (50 psi). The reaction mixture was filtered and concentrated under reduced pressure to give a residue. The residue was purified by column chromatography (SiO₂, petroleum ether/ethyl acetate=10/1 to 1/1). The compound 3-(2,6-dibenzyloxy-3-pyridyl)-1-methyl-indazol-6-amine (1.7 g, 3.8 mmol, 47.8% yield) was obtained as a pink solid.

MS (ESI) m/z: 437.1 [M+H]⁺.

¹H NMR (400 MHz, DMSO-d₆) δ=7.86 (d, J=8.4 Hz, 1H), 7.48-7.45 (m, 2H), 7.42-7.37 (m, 4H), 7.36-7.34 (m, 1H), 7.34-7.29 (m, 4H), 6.55 (d, J=8.0 Hz, 1H), 6.42-6.37 (m, 2H), 5.42 (d, J=10.4 Hz, 4H), 5.32 (s, 2H), 3.84 (s, 3H).

### Step C. Procedure for Preparation of 3-(6-amino-1-methyl-indazol-3-yl)piperidine-2,6-dione

To a solution of 3-(2,6-dibenzyloxy-3-pyridyl)-1-methyl-indazol-6-amine (1.7 g, 3.89 mmol, 1 equiv.) in THE (15 mL) and EtOH (15 mL) was added Pd/C (500 mg, 10% purity), Pd(OH)₂ (500 mg, 20% purity) and acetic acid (233.88 mg, 3.89 mmol, 222.74 μL, 1 equiv.). The mixture was stirred at 50° C. for 24 hours. The reaction mixture was filtered and concentrated under reduced pressure to give a residue. The residue was purified by column chromatography (SiO₂, dichloromethane:methanol=100/1 to 10/1). The compound 3-(6-amino-1-methyl-indazol-3-yl)piperidine-2,6-dione (600 mg, 2.3 mmol, 59.6% yield) was obtained as a pink solid.

MS (ESI) m/z: 259.1 [M+H]⁺

¹H NMR (400 MHz, CDCl₃) δ=7.93 (s, 1H), 7.45 (d, J=8.8 Hz, 1H), 6.58 (d, J=8.8 Hz, 1H), 6.50 (s, 1H), 4.24 (t, J=6.0 Hz, 1H), 3.89 (s, 3H), 3.03-2.95 (m, 1H), 2.70-2.62 (m, 1H), 2.54-2.45 (m, 1H), 2.40-2.31 (m, 1H).

### Step D. Procedure for Preparation of tert-butyl 6-[8-(1,3-benzothiazol-2-ylcarbamoyl)-3,4-dihydro-1H-isoquinolin-2-yl]-3-[3-[2-[1-[2-[[3-(2,6-dioxo-3-piperidyl)-1-methyl-indazol-6-yl]amino]-2-oxo-ethyl]-4-piperidyl]ethoxy]-2-methyl-phenyl]pyridine-2-carboxylate

To a solution of 2-[4-[2-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]ethyl]-1-piperidyl]acetic acid (100 mg, 131.25 μmol, 1 equiv.) and 3-(6-amino-1-methyl-indazol-3-yl)piperidine-2,6-dione (40.68 mg, 157.50 μmol, 1.2 equiv.) in DMF (1 mL) was added DIEA (50.89 mg, 393.74 μmol, 68.58 μL, 3 equiv.) and HATU (64.87 mg, 170.62 μmol, 1.3 equiv.). The mixture was stirred at 25° C. for 2 hours. To the reaction mixture was added H₂O (10 mL), and then the reaction mixture was filtered. The residue was dissolved with DCM (10 mL), dried over Na₂SO₄, filtered, and concentrated under reduced pressure to give tert-butyl 6-[8-(1,3-benzothiazol-2-ylcarbamoyl)-3,4-dihydro-1H-isoquinolin-2-yl]-3-[3-[2-[1-[2-[[3-(2,6-dioxo-3-piperidyl)-1-methyl-indazol-6-yl]amino]-2-oxo-ethyl]-4-piperidyl]ethoxy]-2-methyl-phenyl]pyridine-2-carboxylate (200 mg, crude) as a yellow solid.

MS (ESI) m/z: 501.8 [M+H]⁺/2

### Step E. Procedure for Preparation of 6-[8-(1,3-benzothiazol-2-ylcarbamoyl)-3,4-dihydro-1H-isoquinolin-2-yl]-3-[3-[2-[1-[2-[[3-(2,6-dioxo-3-piperidyl)-1-methyl-indazol-6-yl]amino]-2-oxo-ethyl]-4-piperidyl]ethoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[2-[1-[2-[[3-(2,6-dioxo-3-piperidyl)-1-methyl-indazol-6-yl]amino]-2-oxo-ethyl]-4-piperidyl]ethoxy]-2-methyl-phenyl]pyridine-2-carboxylate (105.70 mg, 105.47 μmol, 1 equiv.) in DCM (1 mL) was added TFA (1.54 g, 13.51 mmol, 1 mL, 128.06 equiv.). The mixture was stirred at 40° C. for 2 hours. The reaction mixture filtered and concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[2-[1-[2-[[3-(2,6-dioxo-3-piperidyl)-1-methyl-indazol-6-yl]amino]-2-oxo-ethyl]-4-piperidyl]ethoxy]-2-methyl-phenyl]pyridine-2-carboxylic acid (26 mg, 26.1 μmol, 24.7% yield, 95% purity) as a white solid.

MS (ESI) m/z: 946.4 [M+H]⁺

¹H NMR (400 MHz, DMSO-d₆) δ=10.85 (s, 1H), 9.96-9.68 (m, 1H), 8.08-7.92 (m, 2H), 7.76 (d, J=7.2 Hz, 1H), 7.80-7.64 (m, 2H), 7.68-7.32 (m, 6H), 7.24-7.16 (m, 1H), 7.12-7.04 (m, 1H), 6.96 (d, J=8.4 Hz, 1H), 6.92 (d, J=8.0 Hz, 1H), 6.60 (d, J=7.2 Hz, 1H), 4.96 (s, 2H), 4.32 (dd, J=9.2, 5.2 Hz, 1H), 4.04 (s, 2H), 3.92 (s, 5H), 3.24-3.08 (m, 1H), 3.06-3.01 (m, 3H), 2.65-2.57 (m, 2H), 2.28-2.12 (m, 4H), 2.08-1.96 (m, 1H), 1.92 (s, 3H), 1.76-1.68 (m, 4H), 1.60-1.52 (m, 1H), 1.48-1.29 (m, 3H).

### Example 188. Preparation of Compound 114

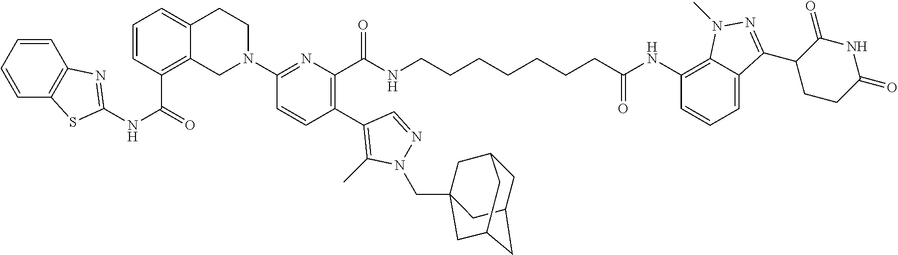 [SMILES: Cc1c(-c2ccc(N3CCc4cccc(C(=O)Nc5nc6ccccc6s5)c4C3)nc2C(=O)NCCCCCCCC(=O)Nc2cccc3c(C4CCC(=O)NC4=O)nn(C)c23)cnn1CC12CC3CC(CC(C3)C1)C2]

### 2-[5-[1-(1-adamantylmethyl)-5-methyl-pyrazol-4-yl]-6-[[8-[[3-(2,6-dioxo-3-piperidyl)-1-methyl-indazol-7-yl]amino]-8-oxo-octyl]carbamoyl]-2-pyridyl]-N-(1,3-benzothiazol-2-yl)-3,4-dihydro-1H-isoquinoline-8-carboxamide

### Step A. Procedure for Preparation of methyl 8-[[3-[1-(1-adamantylmethyl)-5-methyl-pyrazol-4-yl]-6-[8-(1,3-benzothiazol-2-ylcarbamoyl)-3,4-dihydro-1H-isoquinolin-2-yl]pyridine-2-carbonyl]amino]octanoate

A mixture of 3-[1-(1-adamantylmethyl)-5-methyl-pyrazol-4-yl]-6-[8-(1,3-benzothiazol-2-ylcarbamoyl)-3,4-dihydro-1H-isoquinolin-2-yl]pyridine-2-carboxylic acid (1 g, 1.52 mmol, 1 equiv.), methyl 8-aminooctanoate (636.64 mg, 3.04 mmol, 2 equiv., HCl), and HATU (692.58 mg, 1.82 mmol, 1.2 equiv.) in DMF (16 mL) was degassed and purged with N₂ three times, and the mixture was stirred at 25° C. for 5 minutes. After 5 minutes, DIEA (588.53 mg, 4.55 mmol, 793.17 μL, 3 equiv.) was added, and the mixture was stirred at 25° C. for 1 hour under N₂ atmosphere. The reaction mixture was diluted with water (40 mL) and extracted with DCM (50 mL×3). The combined organic layers were washed with brine (30 mL×4), dried over anhydrous sodium sulfate, filtered, and concentrated under reduced pressure to give a residue. The residue was purified by column chromatography (SiO₂, petroleum ether/ethyl acetate=10/1 to 1/1.5). The compound methyl 8-[[3-[1-(1-adamantylmethyl)-5-methyl-pyrazol-4-yl]-6-[8-(1,3-benzothiazol-2-ylcarbamoyl)-3,4-dihydro-1H-isoquinolin-2-yl]pyridine-2-carbonyl]amino]octanoate (710 mg, 846.0 μmol, 55.7% yield, 97% purity) was obtained as a yellow solid.

MS (ESI) m/z: 814.3 [M+H]⁺

### Step B. Procedure for Preparation of 8-[[3-[1-(1-adamantylmethyl)-5-methyl-pyrazol-4-yl]-6-[8-(1,3-benzothiazol-2-ylcarbamoyl)-3,4-dihydro-1H-isoquinolin-2-yl]pyridine-2-carbonyl]amino]octanoic acid

A mixture of methyl 8-[[3-[1-(1-adamantylmethyl)-5-methyl-pyrazol-4-yl]-6-[8-(1,3-benzothiazol-2-ylcarbamoyl)-3,4-dihydro-1H-isoquinolin-2-yl]pyridine-2-carbonyl]amino]octanoate (710 mg, 872.19 μmol, 1 equiv.) and LiOH·H₂O (109.80 mg, 2.62 mmol, 3 equiv.) in THE (9.0 mL) and H₂O (3.0 mL) was degassed and purged with N₂ and stirred at 40° C. for 6 hours under N₂ atmosphere. The crude product was triturated with petroleum ether at 25° C. for 1 hour. The reaction mixture was concentrated under reduced pressure to give a residue. The compound 8-[[3-[1-(1-adamantylmethyl)-5-methyl-pyrazol-4-yl]-6-[8-(1,3-benzothiazol-2-ylcarbamoyl)-3,4-dihydro-1H-isoquinolin-2-yl]pyridine-2-carbonyl]amino]octanoic acid (500 mg, 593.7 μmol, 68.1% yield, 95% purity) was obtained as a yellow solid.

MS (ESI) m/z: 800.4 [M+H]⁺

¹H NMR (400 MHz, DMSO-d₆) δ=13.01-11.96 (m, 1H), 8.06 (t, J=5.6 Hz, 1H), 7.96 (d, J=7=8.0 Hz, 1H), 7.74 (d, J=8.0 Hz, 1H), 7.62 (d, J=6.4 Hz, 1H), 7.47-7.27 (m, 6H), 7.23 (s, 1H), 6.94 (d, J=8.8 Hz, 1H), 5.02 (s, 2H), 3.88 (t, J=6.0 Hz, 2H), 3.02-2.98 (m, 4H), 2.14 (t, J=7.2 Hz, 2H), 2.05 (s, 3H), 1.91 (s, J=5.2 Hz, 3H), 1.69-1.60 (m, 4H), 1.58-1.50 (m, 10H), 1.44-1.39 (m, 2H), 1.30-1.25 (m, 2H), 1.16-1.06 (m, 6H)

### Step C. Procedure for Preparation of 2-[5-[1-(1-adamantylmethyl)-5-methyl-pyrazol-4-yl]-6-[[8-[[3-(2,6-dioxo-3-piperidyl)-1-methyl-indazol-7-yl]amino]-8-oxo-octyl]carbamoyl]-2-pyridyl]-N-(1,3-benzothiazol-2-yl)-3,4-dihydro-1H-isoquinoline-8-carboxamide

A mixture of 8-[[3-[1-(1-adamantylmethyl)-5-methyl-pyrazol-4-yl]-6-[8-(1,3-benzothiazol-2-ylcarbamoyl)-3,4-dihydro-1H-isoquinolin-2-yl]pyridine-2-carbonyl]amino]octanoic acid (40 mg, 50.00 μmol, 1 equiv.), 3-(7-amino-1-methyl-indazol-3-yl)piperidine-2,6-dione (15.50 mg, 60.00 μmol, 1.2 equiv.), and DIPEA (26 mg, 201.17 μmol, 35.04 μL, 4.02 equiv.) in DMF (0.5 mL) was degassed and purged with N₂ three times. The mixture was stirred at 25° C. for 5 minutes. After 5 minutes, HATU (22.81 mg, 60.00 μmol, 1.2 equiv.) was added to the mixture, and the mixture was stirred at 25° C. for 1 hour under N₂ atmosphere. The reaction mixture was filtered and concentrated under reduced pressure to give a residue. The crude product was purified by reverse-phase HPLC. The compound 2-[5-[1-(1-adamantylmethyl)-5-methyl-pyrazol-4-yl]-6-[[8-[[3-(2,6-dioxo-3-piperidyl)-1-methyl-indazol-7-yl]amino]-8-oxo-octyl]carbamoyl]-2-pyridyl]-N-(1,3-benzothiazol-2-yl)-3,4-dihydro-1H-isoquinoline-8-carboxamide (26.2 mg, 25.2 μmol, 50.4% yield, 100% purity) was obtained as a white solid.

MS (ESI) m/z: 1040.6 [M+H]⁺

¹H NMR (400 MHz, DMSO-d₆) δ=12.97-12.69 (m, 1H), 10.89 (s, 1H), 9.80 (s, 1H), 8.09-8.03 (m, 1H), 8.03-7.97 (m, 1H), 7.77 (d, J=7.6 Hz, 1H), 7.59 (t, J=7.2 Hz, 2H), 7.49-7.42 (m, 2H), 7.41-7.37 (m, 1H), 7.37-7.30 (m, 2H), 7.23 (s, 1H), 7.12-7.03 (m, 2H), 6.94 (d, J=8.4 Hz, 1H), 5.00 (s, 2H), 4.40-4.34 (m, 1H), 4.02 (s, 3H), 3.88 (t, J=5.6 Hz, 2H), 3.67 (s, 2H), 3.04-2.97 (m, 4H), 2.69-2.64 (m, 2H), 2.37-2.33 (m, 4H), 2.20-2.15 (m, 1H), 2.05 (s, 3H), 1.91 (s, 3H), 1.68-1.60 (m, 4H), 1.56 (s, 4H), 1.51 (s, 3H), 1.33-1.06 (m, 10H)

### Example 189. Preparation of Compound 240a

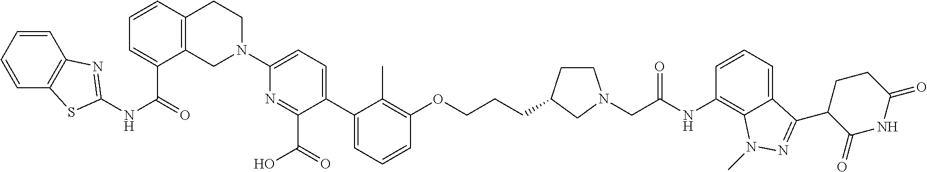 [SMILES: Cc1c(OCCC[C@@H]2CCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[(3R)-1-[2-[[3-(2,6-dioxo-3-piperidyl)-1-methyl-indazol-7-yl]amino]-2-oxo-ethyl]pyrrolidin-3-yl]propoxy]-2-methyl-phenyl]pyridine-2-carboxylic acid

### Step A. Procedure for Preparation of tert-butyl 6-[8-(1,3-benzothiazol-2-ylcarbamoyl)-3,4-dihydro-1H-isoquinolin-2-yl]-3-[3-[3-[(3R)-1-[2-[[3-(2,6-dioxo-3-piperidyl)-1-methyl-indazol-7-yl]amino]-2-oxo-ethyl]pyrrolidin-3-yl]propoxy]-2-methyl-phenyl]pyridine-2-carboxylate

A mixture of 2-[(3R)-3-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]pyrrolidin-1-yl]acetic acid (120 mg, 157.50 μmol, 1 equiv.), 3-(7-amino-1-methyl-indazol-3-yl)piperidine-2,6-dione (48.81 mg, 188.99 μmol, 1.2 equiv.), DIPEA (61.06 mg, 472.49 μmol, 82.30 μL, 3 equiv.), and HATU (59.88 mg, 157.50 μmol, 1.5 equiv.) in DMF (3 mL) was degassed and purged with N₂ and stirred at 40° C. for 6 hours under N₂ atmosphere. The reaction mixture was treated with water (3 mL) and then filtered to give a residue which was dried under reduced pressure. The residue was used in the next step without purification. The compound tert-butyl 6-[8-(1,3-benzothiazol-2-ylcarbamoyl)-3,4-dihydro-1H-isoquinolin-2-yl]-3-[3-[3-[(3R)-1-[2-[[3-(2,6-dioxo-3-piperidyl)-1-methyl-indazol-7-yl]amino]-2-oxo-ethyl]pyrrolidin-3-yl]propoxy]-2-methyl-phenyl]pyridine-2-carboxylate (150 mg, crude) was obtained as a white solid.

MS (ESI) m/z: 1002.7 [M+H]⁺

### Step B. Procedure for Preparation of 6-[8-(1,3-benzothiazol-2-ylcarbamoyl)-3,4-dihydro-1H-isoquinolin-2-yl]-3-[3-[3-[(3R)-1-[2-[[3-(2,6-dioxo-3-piperidyl)-1-methyl-indazol-7-yl]amino]-2-oxo-ethyl]pyrrolidin-3-yl]propoxy]-2-methyl-phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[3-[3-[(3R)-1-[2-[[3-(2,6-dioxo-3-piperidyl)-1-methyl-indazol-7-yl]amino]-2-oxo-ethyl]pyrrolidin-3-yl]propoxy]-2-methyl-phenyl]pyridine-2-carboxylate (150.00 mg, 149.67 μmol, 1 equiv.) in TFA (3 mL) and DCM (3 mL) was degassed and purged with N₂ and stirred at 25° C. for 3 hours under N₂ atmosphere. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[3-[(3R)-1-[2-[[3-(2,6-dioxo-3-piperidyl)-1-methyl-indazol-7-yl]amino]-2-oxo-ethyl]pyrrolidin-3-yl]propoxy]-2-methyl-phenyl]pyridine-2-carboxylic acid (30.8 mg, 31.8 μmol, 21.2% yield, 97.8% purity) as a white solid,

MS (ESI) m/z: 946.4 [M+H]⁺

¹H NMR (400 MHz, DMSO-d₆) δ=12.90-12.82 (m, 1H), 10.90 (s, 1H), 10.02-9.94 (m, 1H), 8.03 (d, J=8.0 Hz, 1H), 7.79 (d, J=8.0 Hz, 1H), 7.61 (d, J=16.8 Hz, 2H), 7.49-7.42 (m, 3H), 7.40-7.33 (m, 2H), 7.27 (d, J=7.6 Hz, 1H), 7.11-7.06 (m, 2H), 6.97-6.95 (m, 1H), 6.89 (d, J=8.4 Hz, 1H), 6.63 (d, J=7.6 Hz, 1H), 4.98 (s, 2H), 4.39-4.36 (m, 1H), 4.08 (s, 3H), 4.01-3.96 (m, 2H), 3.93-3.91 (m, 2H), 3.61-3.41 (m, 4H), 3.04-3.02 (m, 2H), 2.96-2.90 (m, 1H), 2.69-2.60 (m, 2H), 2.42-2.31 (m, 2H), 2.28-2.14 (m, 2H), 2.08-2.00 (m, 1H), 1.90 (s, 3H), 1.76-1.74 (m, 2H), 1.60-1.52 (m, 2H), 1.51-1.42 (m, 1H).

### Example 190. Preparation of Compound 218a

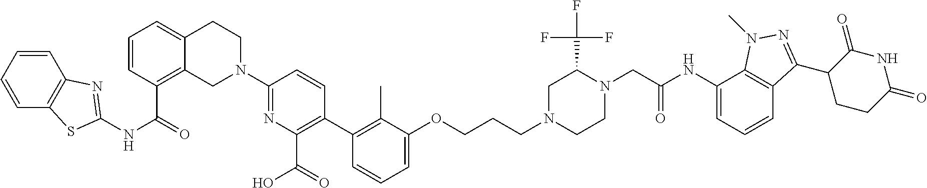 [SMILES: Cc1c(OCCCN2CCN(CC(=O)Nc3cccc4c(C5CCC(=O)NC5=O)nn(C)c34)[C@@H](C(F)(F)F)C2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-((3R)-4-(2-((3-(2,6-dioxopiperidin-3-yl)-1-methyl-1H-indazol-7-yl)amino)-2-oxoethyl)-3-(trifluoromethyl)piperazin-1-yl)propoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(3-((3R)-4-(2-((3-(2,6-dioxopiperidin-3-yl)-1-methyl-11H-indazol-7-yl)amino)-2-oxoethyl)-3-(trifluoromethyl)piperazin-1-yl)propoxy)-2-methylphenyl)picolinate

A mixture of (R)-2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-2-(trifluoromethyl)piperazin-1-yl)acetic acid (75 mg, 88.76 μmol, 1 equiv.), 3-(7-amino-1-methyl-1H-indazol-3-yl)piperidine-2,6-dione (27.51 mg, 106.52 μmol, 1.2 equiv.), and DIEA (34.42 mg, 266.29 μmol, 46.38 μL, 3 equiv.) in DMF (1 mL) was degassed and purged with N₂ and stirred at 25° C. for 5 minutes. After 5 minutes, HATU (40.50 mg, 106.52 μmol, 1.2 equiv.) was added, and the mixture was stirred at 25° C. for 1 hour under N₂ atmosphere. The reaction mixture was diluted with water (20 mL) and extracted with EtOAc (20 mL×3). The combined organic layers were washed with brine (20 mL×6), dried over anhydrous sodium sulfate, filtered, and concentrated under reduced pressure to give tert-butyl 6-(8-(benzo[d]thiazol-2-ylcarbamoyl)-3,4-dihydroisoquinolin-2(1H)-yl)-3-(3-(3-((3R)-4-(2-((3-(2,6-dioxopiperidin-3-yl)-1-methyl-1H-indazol-7-yl)amino)-2-oxoethyl)-3-(trifluoromethyl)piperazin-1-yl)propoxy)-2-methylphenyl)picolinate (100 mg, crude) as a brown solid.

MS (ESI) m/z: 1085.8 [M+H]⁺

### Step B. Procedure for Preparation of 6-(8-(benzo[d]thiazol-2-ylcarbamoyl)-3,4-dihydroisoquinolin-2(1H)-yl)-3-(3-(3-((3R)-4-(2-((3-(2,6-dioxopiperidin-3-yl)-1-methyl-11H-indazol-7-yl)amino)-2-oxoethyl)-3-(trifluoromethyl)piperazin-1-yl)propoxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(3-(3-((3R)-4-(2-((3-(2,6-dioxopiperidin-3-yl)-1-methyl-1H-indazol-7-yl)amino)-2-oxoethyl)-3-(trifluoromethyl)piperazin-1-yl)propoxy)-2-methylphenyl)picolinate (100 mg, 92.15 μmol, 1 equiv.) in TFA (0.5 mL) and DCM (0.5 mL) was degassed and purged with N₂ and stirred at 25° C. for 1 hour under N₂ atmosphere. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(3-((3R)-4-(2-((3-(2,6-dioxopiperidin-3-yl)-1-methyl-1H-indazol-7-yl)amino)-2-oxoethyl)-3-(trifluoromethyl)piperazin-1-yl)propoxy)-2-methylphenyl)picolinic acid (29.2 mg, 26.6 μmol, 28.9% yield, 93.9% purity) as an off-white solid.

MS (ESI) m/z: 1029.4 [M+H]⁺

¹H NMR (400 MHz, DMSO-d₆) δ=13.03-12.32 (m, 2H), 10.90 (s, 1H), 9.79 (s, 1H), 8.14 (s, 1H), 8.03 (d, J=8.0 Hz, 1H), 7.79 (d, J=8.0 Hz, 1H), 7.65-7.57 (m, 2H), 7.50-7.41 (m, 3H), 7.40-7.32 (m, 2H), 7.25-7.19 (m, 1H), 7.12-7.04 (m, 2H), 6.96 (d, J=8.8 Hz, 1H), 6.86 (d, J=8.0 Hz, 1H), 6.63 (d, J=7.6 Hz, 1H), 4.98 (s, 2H), 4.38 (dd, J=10.0, 5.2 Hz, 1H), 4.08 (s, 3H), 4.01 (t, J=5.6 Hz, 2H), 3.91 (t, J=5.6 Hz, 2H), 3.79-3.67 (m, 2H), 3.51-3.44 (m, 1H), 3.02 (t, J=5.6 Hz, 3H), 2.87-2.75 (m, 2H), 2.75-2.62 (m, 2H), 2.61-2.53 (m, 3H), 2.48-2.41 (m, 2H), 2.40-2.31 (m, 1H), 2.21-2.12 (m, 1H), 1.96-1.85 (m, 5H).

### Example 191. Preparation of Compound 161a

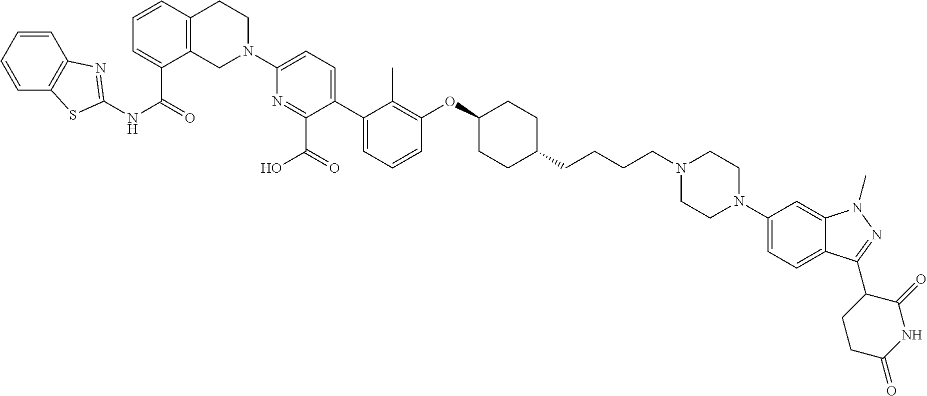 [SMILES: Cc1c(O[C@H]2CC[C@H](CCCCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)butyl)cyclohexyl)oxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)butyl)cyclohexyl)oxy)-2-methylphenyl)picolinate

A mixture of 6-[8-(1,3-benzothiazol-2-ylcarbamoyl)-3,4-dihydro-1H-isoquinolin-2-yl]-3-[2-methyl-3-[4-(4-oxobutyl)cyclohexoxy]phenyl]pyridine-2-carboxylic acid (80 mg, 116.14 μmol, 1 equiv.), 3-(1-methyl-6-piperazin-1-yl-indazol-3-yl)piperidine-2,6-dione (38.02 mg, 116.14 μmol, 1 equiv.), and AcOH (6.97 mg, 116.14 μmol, 6.65 μL, 1 equiv.) in DCM (1 mL) was stirred at 25° C. for 1 hour. NaBH(OAc)₃ (73.84 mg, 348.42 μmol, 3 equiv.) was added, and then the mixture was stirred at 25° C. for 1 hour. The reaction mixture was concentrated under reduced pressure to give tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)butyl)cyclohexyl)oxy)-2-methylphenyl)picolinate (100 mg, crude) as a yellow solid. It was used in the next step without further purification.

MS (ESI) m/z: 529.0 [M+H]⁺.

### Step B. Procedure for Preparation of 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)butyl)cyclohexyl)oxy)-2-methylphenyl)picolinic acid

A solution of tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)butyl)cyclohexyl)oxy)-2-methylphenyl)picolinate (100 mg, 94.67 μmol, 1 equiv.) in TFA (1 mL) and DCM (1 mL) was stirred at 25° C. for 12 hours. The reaction mixture was concentrated under reduced pressure to give a residue. The residue was purified by reverse-phase HPLC to give 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)butyl)cyclohexyl)oxy)-2-methylphenyl)picolinic acid (27.8 mg, 26.0 μmol, 27.5% yield, 93.6% purity) as a white solid.

MS (ESI) m/z: 1000.6 [M+H]⁺.

¹H NMR (400 MHz, DMSO-d₆) δ=10.94 (s, 1H), 8.27 (s, 1H), 8.11 (d, J=7.8 Hz, 1H), 7.87 (d, J=8.0 Hz, 1H), 7.71 (d, J=7.2 Hz, 1H), 7.60-7.40 (m, 6H), 7.18-7.11 (m, 1H), 7.05-6.97 (m, 3H), 6.92 (s, 1H), 6.71 (d, J=7.6 Hz, 1H), 5.06 (s, 2H), 4.37-4.24 (m, 2H), 3.97 (s, 3H), 3.30 (s, 6H), 3.11 (t, J=5.4 Hz, 3H), 2.72-2.66 (m, 2H), 2.48-2.32 (m, 4H), 2.29-2.05 (m, 4H), 1.96 (s, 3H), 1.86 (d, J=11.4 Hz, 2H), 1.56-1.29 (m, 10H), 1.20-1.07 (m, 2H)

### Example 192. Preparation of Compound 184

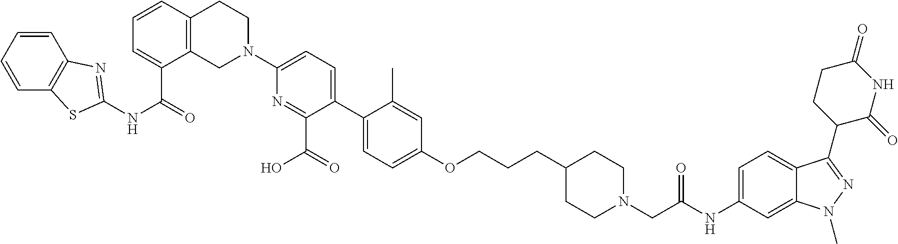 [SMILES: Cc1cc(OCCCC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[4-[3-[1-[2-[[3-(2,6-dioxo-3-piperidyl)-1-methyl-indazol-6-yl]amino]-2-oxo-ethyl]-4-piperidyl]propoxy]-2-methyl-phenyl]pyridine-2-carboxylic acid

### Step A. Procedure for Preparation of tert-butyl 4-[3-(4-bromo-3-methyl-phenoxy)propyl]piperidine-1-carboxylate

A mixture of tert-butyl 4-(3-bromopropyl)piperidine-1-carboxylate (4.00 g, 13.06 mmol, 1 equiv.), 4-bromo-3-methylphenol (3.66 g, 19.59 mmol, 1.5 equiv.) and K₂CO₃ (5.42 g, 39.19 mmol, 3.0 equiv.) in CH₃CN (20 mL) was degassed and purged with N₂ three times, and then the mixture was stirred at 60° C. for 12 hours under N₂ atmosphere. The reaction was diluted with water (100 mL) and extracted with ethyl acetate (100 mL×2). The combined organic layers were washed with brine (100 mL×1), dried over Na₂SO₄, filtered, and concentrated under reduced pressure to give tert-butyl 4-[3-(4-bromo-3-methyl-phenoxy)propyl]piperidine-1-carboxylate (6.7 g, crude) as a colorless oil.

MS (ESI) m/z: 312.2 [M+H−100]⁺.

### Step B. Procedure for Preparation of 4-[3-(4-bromo-3-methyl-phenoxy)propyl]piperidine

A mixture of tert-butyl 4-[3-(4-bromo-3-methyl-phenoxy)propyl]piperidine-1-carboxylate (6.7 g, 16.25 mmol, 1.0 equiv.) in HCl/dioxane (10 mL) was degassed and purged with N₂ three times, and then the mixture was stirred at 25° C. for 2 hours. The reaction mixture was concentrated under reduced pressure to give a residue. The residue was purified by flash silica gel chromatography (eluent of 0˜4% dichloromethane:methanol ether) to give 4-[3-(4-bromo-3-methyl-phenoxy)propyl]piperidine (2.5 g, 8.0 mmol, 49.2% yield) as a white solid.

### Step C. Procedure for Preparation of ethyl 2-[4-[3-(4-bromo-3-methyl-phenoxy)propyl]-1-piperidyl]acetate

A mixture of 4-[3-(4-bromo-3-methyl-phenoxy)propyl]piperidine (2.5 g, 7.17 mmol, 1.0 equiv., HCl), ethyl 2-bromoacetate (1.20 g, 7.17 mmol, 792.91 μL, 1 equiv.), K₂CO₃ (990.88 mg, 7.17 mmol, 1.0 equiv.), and KI (238.02 mg, 1.43 mmol, 0.2 equiv.) in CH₃CN (60 mL) was degassed and purged with N₂ three times. The mixture was then stirred at 25° C. for 1 hour under N₂ atmosphere. The reaction mixture was concentrated under reduced pressure to give a residue. The residue was purified by flash silica gel chromatography (eluent of 0-18% ethyl acetate/petroleum ether) to give ethyl 2-[4-[3-(4-bromo-3-methyl-phenoxy)propyl]-1-piperidyl]acetate (2.3 g, 5.7 mmol, 79.9% yield, 99.3% purity) as yellow oil.

MS (ESI) m/z: 398.2 [M+H]⁺.

### Step D. Procedure for Preparation of tert-butyl 6-[8-(1,3-benzothiazol-2-ylcarbamoyl)-3,4-dihydro-1H-isoquinolin-2-yl]-3-[4-[3-[1-(2-ethoxy2-oxo-ethyl)-4-piperidyl]propoxy]-2-methyl-phenyl]pyridine-2-carboxylate

A mixture of ethyl 2-[4-[3-(4-bromo-3-methyl-phenoxy)propyl]-1-piperidyl]acetate (2.3 g, 5.77 mmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (3.18 g, 5.20 mmol, 0.9 equiv.), K₂CO₃ (1.5 M, 11.55 mL, 3.0 equiv.), and Ad2nBuP Pd G3 (841.01 mg, 1.15 mmol, 0.2 equiv.) in dioxane (20 mL) was degassed and purged with N₂ three times. The mixture was then stirred at 80° C. for 1 hour under N₂ atmosphere. The reaction mixture was concentrated under reduced pressure to give a residue. The residue was purified by flash silica gel chromatography (eluent of 0˜59% ethyl acetate/petroleum ether) to give tert-butyl 6-[8-(1,3-benzothiazol-2-ylcarbamoyl)-3,4-dihydro-1H-isoquinolin-2-yl]-3-[4-[3-[1-(2-ethoxy2-oxo-ethyl)-4-piperidyl]propoxy]-2-methyl-phenyl]pyridine-2-carboxylate (3.2 g, 3.9 mmol, 68.9% yield) as a yellow oil.

MS (ESI) m/z: 804.5 [M+H]⁺.

### Step E. Procedure for Preparation of 2-[4-[3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]propyl]-1-piperidyl]acetic acid

A mixture of tert-butyl 6-[8-(1,3-benzothiazol-2-ylcarbamoyl)-3,4-dihydro-1H-isoquinolin-2-yl]-3-[4-[3-[1-(2-ethoxy2-oxo-ethyl)-4-piperidyl]propoxy]-2-methyl-phenyl]pyridine-2-carboxylate (3.2 g, 3.98 mmol, 1.0 equiv.) and LiOH·H₂O (1 M, 11.94 mL, 3.0 equiv.) in THF (20 mL) was degassed and purged with N₂ three times. The mixture was then stirred at 25° C. for 2 hours under N₂ atmosphere. The reaction mixture was concentrated under reduced pressure to give a residue. Then HCl (1 M) was added to the residue to adjust the pH to 4-5. A white precipitate formed, and the solid was collected by filtration and dried to give 2-[4-[3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]propyl]-1-piperidyl]acetic acid (2.6 g, 3.2 mmol, 82.7% yield, 98.3% purity) as a white solid.

MS (ESI) m/z: 776.5 [M+H]⁺.

### Step F. Procedure for Preparation of tert-butyl 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-1-methyl-1H-indazol-6-yl)amino)-2-oxoethyl)piperidin-4-yl)propoxy)-2-methylphenyl)picolinate

2-[4-[3-[4-[6-[8-(1,3-Benzothiazol-2-ylcarbamoyl)-3,4-dihydro-1H-isoquinolin-2-yl]-2-tert-butoxycarbonyl-3-pyridyl]-3-methyl-phenoxy]propyl]-1-piperidyl]acetic acid (50 mg, 64 μmol, 1 equiv.) was dissolved in DCM (5 mL). To the mixture was added HATU (27 mg, 71 μmol, 1.1 equiv.), followed by 3-(6-amino-1-methyl-1H-indazol-3-yl)piperidine-2,6-dione (20 mg, 77 μmol, 1.2 equiv.) and DIEA (25 mg, 34 μL, 0.19 mmol, 3 equiv.). The mixture was then stirred at 25° C. overnight. The solvents were removed. The resulting residue was used in the next step without further purification.

### Step G. Procedure for Preparation of 6-[8-(1,3-benzothiazol-2-ylcarbamoyl)-3,4-dihydro-1H-isoquinolin-2-yl]-3-[4-[3-[1-[2-[[3-(2,6-dioxo-3-piperidyl)-1-methyl-indazol-6-yl]amino]-2-oxo-ethyl]-4-piperidyl]propoxy]-2-methyl-phenyl]pyridine-2-carboxylic acid

To a solution of tert-butyl 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-1-methyl-1H-indazol-6-yl)amino)-2-oxoethyl)piperidin-4-yl)propoxy)-2-methylphenyl)picolinate (65 mg, 64 μmol, 1 equiv.) in DCM (4 mL) was added TFA (1 mL). The reaction mixture was stirred at 25° C. over night. The solvents were then removed, and the residue was purified by prep-HPLC to give 6-[8-(1,3-benzothiazol-2-ylcarbamoyl)-3,4-dihydro-1H-isoquinolin-2-yl]-3-[4-[3-[1-[2-[[3-(2,6-dioxo-3-piperidyl)-1-methyl-indazol-6-yl]amino]-2-oxo-ethyl]-4-piperidyl]propoxy]-2-methyl-phenyl]pyridine-2-carboxylic acid (32 mg, 33 μmol, 52% yield) as a white solid.

MS (ESI) m/z: 960 [M+H]⁺.

¹H NMR (400 MHz, DMSO-d₆) δ=12.45-12.85 (m, 2H), 10.88 (s, 1H), 8.03 (d, J=6.4 Hz, 2H), 7.79 (d, J=8.0 Hz, 1H), 7.66-7.59 (m, 2H), 7.50-7.41 (m, 3H), 7.40-7.32 (m, 2H), 7.20 (d, J=8.8 Hz, 1H), 6.97-6.80 (m, 2H), 6.78 (s, 1H), 6.75-6.70 (m, 1H), 4.97 (s, 2H), 4.32 (dd, J=5.2, 9.6 Hz, 1H), 3.97-3.89 (m, 7H), 3.02 (t, J=6.4 Hz, 2H), 2.65-2.63 (m, 2H), 2.51-2.48 (m, 4H), 2.38-2.29 (m, 2H), 2.20-2.12 (m, 1H), 2.03 (s, 3H), 1.76-1.73 (m, 4H), 1.39 (s, 5H)

### Example 193. Preparation of Compound 262b

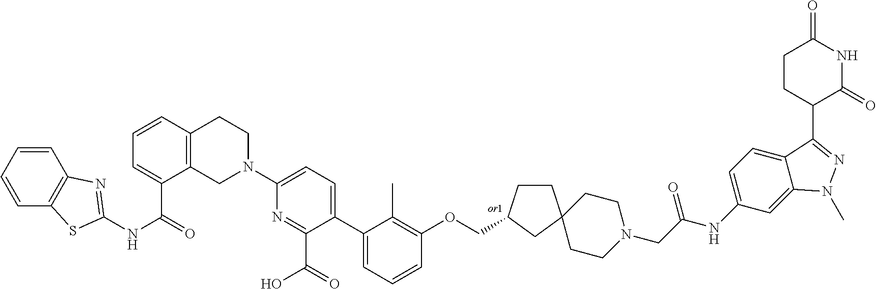 [SMILES: Cc1c(OC[C@@H]2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((2R)-8-(2-((3-(2,6-dioxopiperidin-3-yl)-1-methyl-1H-indazol-6-yl)amino)-2-oxoethyl)-8-azaspiro[4.5]decan-2-yl)methoxy)-2-methylphenyl)picolinic acid

### [rel-6-(8-(benzo[d]thiazol-2-ylcarbamoyl)-3,4-dihydroisoquinolin-2(1H)-yl)-3-(3-(((2R)-8-(2-((3-(2,6-dioxopiperidin-3-yl)-1-methyl-1H-indazol-6-yl)amino)-2-oxoethyl)-8-azaspiro[4.5]decan-2-yl)methoxy)-2-methylphenyl)picolinic acid]

### Step A. Procedure for Preparation of tert-butyl 3-[(3-bromo-2-methyl-phenoxy)methyl]-8-azaspiro[4.5]decane-8-carboxylate

A mixture of tert-butyl 3-(hydroxymethyl)-8-azaspiro[4.5]decane-8-carboxylate (1 g, 3.71 mmol, 1.2 equiv.), 3-bromo-2-methyl-phenol (578.60 mg, 3.09 mmol, 1 equiv.), and 2-(tributyl-λ5-phosphanylidene)acetonitrile (1.49 g, 6.19 mmol, 2 equiv.) in toluene (5 mL) was stirred at 120° C. for 12 hours under N₂. The mixture was concentrated to remove toluene. The residue was purified by flash silica gel chromatography (eluent of 0˜20% ethyl acetate/petroleum ether) to give tert-butyl 3-[(3-bromo-2-methyl-phenoxy)methyl]-8-azaspiro[4.5]decane-8-carboxylate (1.1 g, 2.3 mmol, 74.6% yield, 92% purity) as a colorless oil.

¹H NMR (400 MHz, CDCl₃-d) 6=7.13 (d, J=8.0 Hz, 1H), 6.98 (t, J=8.0 Hz, 1H), 6.74 (d, J=8.0 Hz, 1H), 3.85 (d, J=6.4 Hz, 2H), 3.43-3.33 (m, 4H), 2.55-2.45 (m, 1H), 2.34-2.30 (m, 3H), 1.95-1.78 (m, 2H), 1.62-1.50 (m, 6H), 1.46 (s, 9H), 1.30-1.19 (m, 2H)

### Step B. Procedure for Preparation of tert-butyl (3R)-3-[(3-bromo-2-methyl-phenoxy)methyl]-8-azaspiro[4.5]decane-8-carboxylate

The stereoisomers of tert-butyl 3-[(3-bromo-2-methyl-phenoxy)methyl]-8-azaspiro[4.5]decane-8-carboxylate were separated by SFC to give tert-butyl (3S)-3-[(3-bromo-2-methyl-phenoxy)methyl]-8-azaspiro[4.5]decane-8-carboxylate (peak 1) (400 mg, 912.41 μmol, 36.36% yield), tert-butyl (3R)-3-[(3-bromo-2-methyl-phenoxy)methyl]-8-azaspiro[4.5]decane-8-carboxylate (peak 2) (300 mg, 684.3 μmol, 27.2% yield) was obtained as a white oil.

¹H NMR (400 MHz, DMSO-d₆) δ=7.17-7.12 (m, 1H), 7.11-7.05 (m, 1H), 6.95 (d, J=8.0 Hz, 1H), 3.88 (d, J=6.4 Hz, 2H), 3.28 (d, J=5.2 Hz, 4H), 2.47-2.41 (m, 1H), 2.23 (s, 3H), 1.84 (d, J=8.0 Hz, 1H), 1.76 (dd, J=8.0, 12.8 Hz, 1H), 1.53-1.45 (m, 3H), 1.38 (s, 13H), 1.19 (dd, J=9.6, 12.4 Hz, 1H)

¹H NMR (400 MHz, DMSO-d₆) δ=7.18-7.12 (m, 1H), 7.11-7.04 (m, 1H), 6.96 (d, J=8.0 Hz, 1H), 3.88 (d, J=6.4 Hz, 2H), 3.31-3.26 (m, 4H), 2.47-2.41 (m, 1H), 1.89-1.81 (m, 1H), 1.80-1.72 (m, 1H), 1.54-1.46 (m, 3H), 1.41-1.34 (m, 13H), 1.19 (dd, J=9.2, 12.8 Hz, 1H)

### Step C. Procedure for Preparation of (3R)-3-[(3-bromo-2-methyl-phenoxy)methyl]-8-azaspiro[4.5]decane

A solution of tert-butyl (3R)-3-[(3-bromo-2-methyl-phenoxy)methyl]-8-azaspiro[4.5]decane-8-carboxylate (300 mg, 684.31 μmol, 1 equiv.) in HCl/dioxane (1.5 mL) and DCM (1.5 mL) was stirred at 25° C. for 0.5 hours. The reaction mixture was concentrated under reduced pressure to give (3R)-3-[(3-bromo-2-methyl-phenoxy)methyl]-8-azaspiro[4.5]decane (260 mg, crude) as a white solid.

MS (ESI) m/z: 337.8 [M+H]⁺.

### Step D. Procedure for Preparation of ethyl 2-[(3R)-3-[(3-bromo-2-methyl-phenoxy)methyl]-8-azaspiro[4.5]decan-8-yl]acetate

A mixture of (3R)-3-[(3-bromo-2-methyl-phenoxy)methyl]-8-azaspiro[4.5]decane (260 mg, 693.81 μmol, 1 equiv. HCl), ethyl 2-bromoacetate (104.28 mg, 624.43 μmol, 69.06 uL, 0.9 equiv.), KI (11.52 mg, 69.38 μmol, 0.1 equiv.), and K₂CO₃ (287.67 mg, 2.08 mmol, 3 equiv.) in CH₃CN (2.6 mL) was stirred at 60° C. for 0.5 hours. The reaction mixture was concentrated under reduced pressure to give a residue. The residue was purified by flash silica gel chromatography (eluent of 0˜22% ethyl acetate/petroleum ether) to give ethyl 2-[(3R)-3-[(3-bromo-2-methyl-phenoxy)methyl]-8-azaspiro[4.5]decan-8-yl]acetate (220 mg, 508.0 μmol, 73.2% yield, 98% purity) as a colorless oil.

MS (ESI) m/z: 424.2 [M+H]⁺.

¹H NMR (400 MHz, CDCl₃) δ=7.14 (d, J=8.0 Hz, 1H), 6.99 (t, J=8.0 Hz, 1H), 6.75 (d, J=8.0 Hz, 1H), 4.20 (q, J=7.2 Hz, 2H), 3.89-3.81 (m, 2H), 3.25 (s, 2H), 2.58 (d, J=1.6 Hz, 4H), 2.49 (s, 1H), 2.32 (s, 3H), 1.91 (d, J=6.0 Hz, 1H), 1.82 (dd, J=8.0, 12.8 Hz, 1H), 1.60-1.45 (m, 7H), 1.29 (t, J=7.2 Hz, 3H), 1.23 (dd, J=9.6, 13.2 Hz, 1H)

### Step E. Procedure for Preparation of tert-butyl 6-[8-(1,3-benzothiazol-2-ylcarbamoyl)-3,4-dihydro-1H-isoquinolin-2-yl]-3-[3-[[(3R)-8-(2-ethoxy-2-oxo-ethyl)-8-azaspiro[4.5]decan-3-yl]methoxy]-2-methyl-phenyl]pyridine-2-carboxylate

Ethyl 2-[(3R)-3-[(3-bromo-2-methyl-phenoxy)methyl]-8-azaspiro[4.5]decan-8-yl]acetate (120 mg, 282.77 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (207.85 mg, 339.33 μmol, 1.2 equiv.), [2-(2-aminophenyl)phenyl]palladium(1+); bis(1-adamantyl)-butyl-phosphane; methanesulfonate (20.59 mg, 28.28 μmol, 0.1 equiv.), and KF (49.29 mg, 848.32 μmol, 19.87 uL, 3 equiv.) were taken up into a microwave tube in 1,4-dioxane (2.3 mL) and H₂O (0.2 mL). The sealed tube was heated at 100° C. for 1 hour under microwave. The reaction mixture was concentrated under reduced pressure to give a residue. The residue was purified by flash silica gel chromatography (eluent of 0˜13% dichloromethane:methanol) to give tert-butyl 6-[8-(1,3-benzothiazol-2-ylcarbamoyl)-3,4-dihydro-1H-isoquinolin-2-yl]-3-[3-[[(3R)-8-(2-ethoxy-2-oxo-ethyl)-8-azaspiro[4.5]decan-3-yl]methoxy]-2-methyl-phenyl]pyridine-2-carboxylate (260 mg, crude) as a yellow oil.

### Step F. Procedure for Preparation of 2-[(3R)-3-[[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]methyl]-8-azaspiro[4.5]decan-8-yl]acetic acid

To a solution of tert-butyl 6-[8-(1,3-benzothiazol-2-ylcarbamoyl)-3,4-dihydro-1H-isoquinolin-2-yl]-3-[3-[[(3R)-8-(2-ethoxy-2-oxo-ethyl)-8-azaspiro[4.5]decan-3-yl]methoxy]-2-methyl-phenyl]pyridine-2-carboxylate (260 mg, 313.24 μmol, 1 equiv.) in THE (3 mL) and H₂O (1 mL) was added LiOH·H₂O (65.72 mg, 1.57 mmol, 5 equiv.). The mixture was stirred at 25° C. for 16 hours. The reaction mixture was concentrated under reduced pressure to give a residue. The residue was treated with water (6 mL), and its pH was adjusted to 3 by progressively adding diluted HCl (1M). The resulting mixture was filtered to give 2-[(3R)-3-[[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]methyl]-8-azaspiro[4.5]decan-8-yl]acetic acid (170 mg, 165.1 μmol, 52.7% yield, 77.9% purity) as a white solid.

MS (ESI) m/z: 802.3 [M+H]⁺.

### Step G. Procedure for Preparation of tert-butyl 6-[8-(1,3-benzothiazol-2-ylcarbamoyl)-3,4-dihydro-1H-isoquinolin-2-yl]-3-[3-[[(3R)-8-[2-[[3-(2,6-dioxo-3-piperidyl)-1-methyl-indazol-6-yl]amino]-2-oxo-ethyl]-8-azaspiro[4.5]decan-3-yl]methoxy]-2-methyl-phenyl]pyridine-2-carboxylate

To a solution of 2-[(3R)-3-[[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]methyl]-8-azaspiro[4.5]decan-8-yl]acetic acid (170 mg, 211.97 μmol, 1 equiv.), and 3-(6-amino-1-methyl-indazol-3-yl)piperidine-2,6-dione (65.70 mg, 254.37 μmol, 1.2 equiv.) in DMF (1.7 mL) was added HATU (120.90 mg, 317.96 μmol, 1.5 equiv.) and DIEA (82.19 mg, 635.92 μmol, 110.76 μL, 3 equiv.). The mixture was stirred at 25° C. for 16 hours. The reaction mixture was diluted with water (10 mL) and filtered to give tert-butyl 6-[8-(1,3-benzothiazol-2-ylcarbamoyl)-3,4-dihydro-1H-isoquinolin-2-yl]-3-[3-[[(3R)-8-[2-[[3-(2,6-dioxo-3-piperidyl)-1-methyl-indazol-6-yl]amino]-2-oxo-ethyl]-8-azaspiro[4.5]decan-3-yl]methoxy]-2-methyl-phenyl]pyridine-2-carboxylate (170 mg, 117.4 μmol, 55.4% yield, 72% purity) as a pink solid.

MS (ESI) m/z: 521.9 [½M+H]⁺

### Step H. Procedure for Preparation of 6-(8-(benzo[d]thiazol-2-ylcarbamoyl)-3,4-dihydroisoquinolin-2(1H)-yl)-3-(3-(((2R)-8-(2-((3-(2,6-dioxopiperidin-3-yl)-1-methyl-1H-indazol-6-yl)amino)-2-oxoethyl)-8-azaspiro[4.5]decan-2-yl)methoxy)-2-methylphenyl)picolinic acid [rel-6-(8-(benzo[d]thiazol-2-ylcarbamoyl)-3,4-dihydroisoquinolin-2(1H)-yl)-3-(3-(((2R)-8-(2-((3-(2,6-dioxopiperidin-3-yl)-1-methyl-1H-indazol-6-yl)amino)-2-oxoethyl)-8-azaspiro[4.5]decan-2-yl)methoxy)-2-methylphenyl)picolinic acid]

A solution of tert-butyl 6-[8-(1,3-benzothiazol-2-ylcarbamoyl)-3,4-dihydro-1H-isoquinolin-2-yl]-3-[3-[[(3R)-8-[2-[[3-(2,6-dioxo-3-piperidyl)-1-methyl-indazol-6-yl]amino]-2-oxo-ethyl]-8-azaspiro[4.5]decan-3-yl]methoxy]-2-methyl-phenyl]pyridine-2-carboxylate (170.00 mg, 163.11 μmol, 1 equiv.) in TFA (1 mL) and DCM (2 mL) was stirred at 25° C. for 2 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((2R)-8-(2-((3-(2,6-dioxopiperidin-3-yl)-1-methyl-1H-indazol-6-yl)amino)-2-oxoethyl)-8-azaspiro[4.5]decan-2-yl)methoxy)-2-methylphenyl)picolinic acid [rel-6-(8-(benzo[d]thiazol-2-ylcarbamoyl)-3,4-dihydroisoquinolin-2(1H)-yl)-3-(3-(((2R)-8-(2-((3-(2,6-dioxopiperidin-3-yl)-1-methyl-1H-indazol-6-yl)amino)-2-oxoethyl)-8-azaspiro[4.5]decan-2-yl)methoxy)-2-methylphenyl)picolinic acid] (36.2 mg, 34.4 μmol, 21.1% yield, 93.9% purity) was obtained as an off-white solid.

MS (ESI) m/z: 986.4 [M+H]⁺.

¹H NMR (400 MHz, DMSO-d₆) δ=10.87 (s, 1H), 9.87-9.80 (m, 1H), 8.16 (s, 1H), 8.06-8.00 (m, 2H), 7.78 (d, J=8.0 Hz, 1H), 7.62 (d, J=8.4 Hz, 2H), 7.49-7.41 (m, 3H), 7.36 (d, J=7.6, 10.2 Hz, 2H), 7.22 (dd, J=1.6, 8.8 Hz, 1H), 7.11-7.05 (m, 1H), 6.95 (d, J=8.8 Hz, 1H), 6.87 (d, J=8.4 Hz, 1H), 6.63 (d, J=7.6 Hz, 1H), 4.98 (s, 2H), 4.32 (dd, J=5.2, 9.6 Hz, 1H), 3.92 (s, 4H), 3.91-3.83 (m, 3H), 3.13 (s, 4H), 3.02 (t, J=5.6 Hz, 2H), 2.74-2.57 (m, 3H), 2.56 (s, 2H), 2.38-2.30 (m, 1H), 2.21-2.12 (m, 1H), 1.90 (s, 3H), 1.87-1.72 (m, 2H), 1.60-1.46 (m, 7H), 1.26-1.20 (m, 1H)

### Example 194. Preparation of Compound 262a

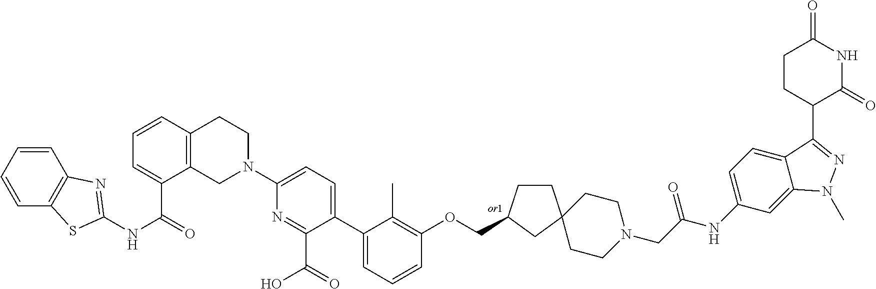 [SMILES: Cc1c(OC[C@H]2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[[(3S)-8-[2-[[3-(2,6-dioxo-3-piperidyl)-1-methyl-indazol-6-yl]amino]-2-oxo-ethyl]-8-azaspiro[4.5]decan-3-yl]methoxy]-2-methyl-phenyl]pyridine-2-carboxylic acid

### [rel-6-(8-(benzo[d]thiazol-2-ylcarbamoyl)-3,4-dihydroisoquinolin-2(1H)-yl)-3-(3-(((2S)-8-(2-((3-(2,6-dioxopiperidin-3-yl)-1-methyl-1H-indazol-6-yl)amino)-2-oxoethyl)-8-azaspiro[4.5]decan-2-yl)methoxy)-2-methylphenyl)picolinic acid]

### Step A. Procedure for Preparation of (3S)-3-[(3-bromo-2-methyl-phenoxy)methyl]-8-azaspiro[4.5]decane

A solution of tert-butyl (3S)-3-[(3-bromo-2-methyl-phenoxy)methyl]-8-azaspiro[4.5]decane-8-carboxylate (400 mg, 912.41 μmol, 1 equiv.) in HCl/dioxane (2 mL) and DCM (2 mL) was stirred at 25° C. for 0.5 hours. The reaction mixture was concentrated under reduced pressure to give (3S)-3-[(3-bromo-2-methyl-phenoxy)methyl]-8-azaspiro[4.5]decane (320 mg, crude) as a white solid.

MS (ESI) m/z: 338.0 [M+H]⁺.

### Step B. Procedure for Preparation of ethyl 2-[(3S)-3-[(3-bromo-2-methyl-phenoxy)methyl]-8-azaspiro[4.5]decan-8-yl]acetate

A mixture of (3S)-3-[(3-bromo-2-methyl-phenoxy)methyl]-8-azaspiro[4.5]decane (320 mg, 853.92 μmol, 1 equiv. HCl.), ethyl 2-bromoacetate (128.34 mg, 768.53 μmol, 85.00 μL, 0.9 equiv.), KI (14.18 mg, 85.39 μmol, 0.1 equiv.), and K₂CO₃ (354.05 mg, 2.56 mmol, 3 equiv.) in CH₃CN (3.2 mL) was stirred at 60° C. for 2.5 hours. The reaction mixture was concentrated under reduced pressure to give a residue. The residue was purified by flash silica gel chromatography (eluent of 0˜22% ethyl acetate/petroleum ether) to give ethyl 2-[(3S)-3-[(3-bromo-2-methyl-phenoxy)methyl]-8-azaspiro[4.5]decan-8-yl]acetate (230 mg, 536.6 μmol, 62.8% yield, 99% purity) was obtained as a colorless oil.

MS (ESI) m/z: 426.2 [M+H]⁺.

¹H NMR (400 MHz, DMSO-d₆) δ=7.16-7.12 (m, 1H), 7.11-7.05 (m, 1H), 6.95 (d, J=8.0 Hz, 1H), 4.10-4.03 (m, 2H), 3.92-3.83 (m, 2H), 3.16 (s, 2H), 2.43 (d, J=5.2, 10.4 Hz, 4H), 2.23 (s, 3H), 1.84-1.77 (m, 1H), 1.72 (dd, J=8.0, 12.8 Hz, 1H), 1.50-1.38 (m, 7H), 1.20-1.14 (m, 4H)

### Step C. Procedure for Preparation of tert-butyl 6-[8-(1,3-benzothiazol-2-ylcarbamoyl)-3,4-dihydro-1H-isoquinolin-2-yl]-3-[3-[[(3S)-8-(2-ethoxy-2-oxo-ethyl)-8-azaspiro[4.5]decan-3-yl]methoxy]-2-methyl-phenyl]pyridine-2-carboxylate

Ethyl 2-[(3S)-3-[(3-bromo-2-methyl-phenoxy)methyl]-8-azaspiro[4.5]decan-8-yl]acetate (230 mg, 541.98 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (398.38 mg, 650.37 μmol, 1.2 equiv.), [2-(2-aminophenyl)phenyl]palladium(1⁺); bis(1-adamantyl)-butyl-phosphane; methanesulfonate (39.47 mg, 54.20 μmol, 0.1 equiv.), and KF (94.46 mg, 1.63 mmol, 38.09 μL, 3 equiv.) were taken up into a microwave tube in 1,4-dioxane (2.3 mL) and H₂O (0.2 mL). The sealed tube was heated at 100° C. for 1 hour under microwave. The reaction mixture was concentrated under reduced pressure to give a residue. The residue was purified by flash silica gel chromatography (eluent of 0˜13% dichloromethane: methanol) to give tert-butyl 6-[8-(1,3-benzothiazol-2-ylcarbamoyl)-3,4-dihydro-1H-isoquinolin-2-yl]-3-[3-[[(3S)-8-(2-ethoxy-2-oxo-ethyl)-8-azaspiro[4.5]decan-3-yl]methoxy]-2-methyl-phenyl]pyridine-2-carboxylate (480 mg, 414.6 μmol, 76.5% yield, 71.7% purity) as a yellow oil.

MS (ESI) m/z: 426.0 [M+H]⁺.

### Step D. Procedure for Preparation of 2-[(3S)-3-[[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]methyl]-8-azaspiro[4.5]decan-8-yl]acetic acid

To a solution of tert-butyl 6-[8-(1,3-benzothiazol-2-ylcarbamoyl)-3,4-dihydro-1H-isoquinolin-2-yl]-3-[3-[[(3S)-8-(2-ethoxy-2-oxo-ethyl)-8-azaspiro[4.5]decan-3-yl]methoxy]-2-methyl-phenyl]pyridine-2-carboxylate (200.00 mg, 240.95 μmol, 1 equiv.) in THE (1 mL) and H₂O (0.3 mL) was added LiOH·H₂O (50.56 mg, 1.20 mmol, 5 equiv.). The mixture was stirred at 25° C. for 16 hours. The reaction mixture was concentrated under reduced pressure to give a residue. The residue was treated with water (6 mL). The pH was adjusted to around 3 by progressively adding dilute HCl. The mixture was filtered to give 2-[(3S)-3-[[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]methyl]-8-azaspiro[4.5]decan-8-yl]acetic acid (100 mg, 89.8 μmol, 37.2% yield, 72% purity) as a white solid.

MS (ESI) m/z: 802.6 [M+H]⁺.

### Step E. Procedure for Preparation of tert-butyl 6-[8-(1,3-benzothiazol-2-ylcarbamoyl)-3,4-dihydro-1H-isoquinolin-2-yl]-3-[3-[[(3S)-8-[2-[[3-(2,6-dioxo-3-piperidyl)-1-methyl-indazol-6-yl]amino]-2-oxo-ethyl]-8-azaspiro[4.5]decan-3-yl]methoxy]-2-methyl-phenyl]pyridine-2-carboxylate

To a solution of 3-(6-amino-1-methyl-indazol-3-yl)piperidine-2,6-dione (38.65 mg, 149.63 μmol, 1.2 equiv.), 2-[(3S)-3-[[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]methyl]-8-azaspiro[4.5]decan-8-yl]acetic acid (100.00 mg, 124.69 μmol, 1 equiv.) in DMF (1 mL) was added HATU (71.12 mg, 187.04 μmol, 1.5 equiv.) and DIEA (48.35 mg, 374.08 μmol, 65.16 μL, 3 equiv.). The mixture was stirred at 25° C. for 16 hours. The reaction mixture was diluted with water (10 mL) and filtered to give tert-butyl 6-[8-(1,3-benzothiazol-2-ylcarbamoyl)-3,4-dihydro-1H-isoquinolin-2-yl]-3-[3-[[(3S)-8-[2-[[3-(2,6-dioxo-3-piperidyl)-1-methyl-indazol-6-yl]amino]-2-oxo-ethyl]-8-azaspiro[4.5]decan-3-yl]methoxy]-2-methyl-phenyl]pyridine-2-carboxylate (160 mg, 89.3 μmol, 71.6% yield, 58.2% purity) as a pink solid.

MS (ESI) m/z: 521.9 [½M+H]⁺

### Step F. Procedure for Preparation of 6-[8-(1,3-benzothiazol-2-ylcarbamoyl)-3,4-dihydro-1H-isoquinolin-2-yl]-3-[3-[[(3S)-8-[2-[[3-(2,6-dioxo-3-piperidyl)-1-methyl-indazol-6-yl]amino]-2-oxo-ethyl]-8-azaspiro[4.5]decan-3-yl]methoxy]-2-methyl-phenyl]pyridine-2-carboxylic acid [rel-6-(8-(benzo[d]thiazol-2-ylcarbamoyl)-3,4-dihydroisoquinolin-2(1H)-yl)-3-(3-(((2S)-8-(2-((3-(2,6-dioxopiperidin-3-yl)-1-methyl-1H-indazol-6-yl)amino)-2-oxoethyl)-8-azaspiro[4.5]decan-2-yl)methoxy)-2-methylphenyl)picolinic acid]

To a solution of tert-butyl 6-[8-(1,3-benzothiazol-2-ylcarbamoyl)-3,4-dihydro-1H-isoquinolin-2-yl]-3-[3-[[(3S)-8-[2-[[3-(2,6-dioxo-3-piperidyl)-1-methyl-indazol-6-yl]amino]-2-oxo-ethyl]-8-azaspiro[4.5]decan-3-yl]methoxy]-2-methyl-phenyl]pyridine-2-carboxylate (160.00 mg, 153.51 μmol, 1 equiv.) in TFA (1 mL) and DCM (2 mL). The mixture was stirred at 25° C. for 2 hours. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[[(3S)-8-[2-[[3-(2,6-dioxo-3-piperidyl)-1-methyl-indazol-6-yl]amino]-2-oxo-ethyl]-8-azaspiro[4.5]decan-3-yl]methoxy]-2-methyl-phenyl]pyridine-2-carboxylic acid [rel-6-(8-(benzo[d]thiazol-2-ylcarbamoyl)-3,4-dihydroisoquinolin-2(1H)-yl)-3-(3-(((2S)-8-(2-((3-(2,6-dioxopiperidin-3-yl)-1-methyl-1H-indazol-6-yl)amino)-2-oxoethyl)-8-azaspiro[4.5]decan-2-yl)methoxy)-2-methylphenyl)picolinic acid] (20.9 mg, 20.5 μmol, 13.3% yield, 96.8% purity) was obtained as an off-white solid.

MS (ESI) m/z: 493.9 [½M+H]⁺.

¹H NMR (400 MHz, DMSO-d₆) δ=10.87 (s, 1H), 9.87 (s, 1H), 8.05-8.00 (m, 2H), 7.79 (d, J=8.0 Hz, 1H), 7.65-7.59 (m, 2H), 7.50-7.41 (m, 3H), 7.40-7.32 (m, 2H), 7.21 (dd, J=1.6, 8.8 Hz, 1H), 7.11-7.05 (m, 1H), 6.96 (d, J=8.8 Hz, 1H), 6.87 (d, J=8.4 Hz, 1H), 6.62 (d, J=7.6 Hz, 1H), 4.98 (s, 2H), 4.32 (dd, J=5.2, 9.6 Hz, 1H), 3.92 (s, 4H), 3.91-3.84 (m, 3H), 3.17 (s, 4H), 3.02 (t, J=5.6 Hz, 2H), 2.71-2.60 (m, 3H), 2.55-2.53 (m, 2H), 2.37-2.30 (m, 1H), 2.22-2.12 (m, 1H), 1.90 (s, 3H), 1.87-1.75 (m, 2H), 1.60-1.45 (m, 7H), 1.23 (t, J=11.2 Hz, 1H)

### Example 195. Preparation of Compound 249

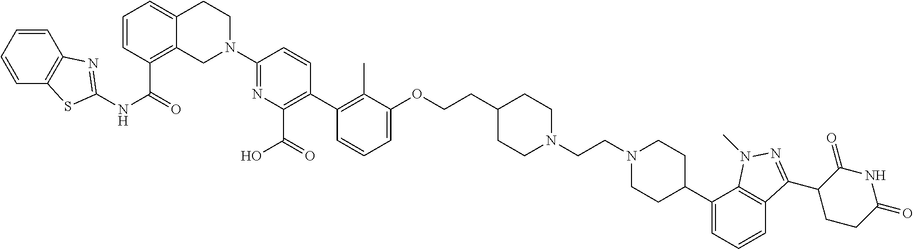 [SMILES: Cc1c(OCCC2CCN(CCN3CCC(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3, 4-dihydro-1H-isoquinolin-2-yl]-3-[3-[2-[1-[2-[4-[3-(2, 6-dioxo-3-piperidyl)-1-methyl-indazol-7-yl]-1-piperidyl]ethyl]-4-piperidyl]ethoxy]-2-methyl-phenyl] pyridine-2-carboxylic acid

### Step A. Procedure for Preparation of tert-butyl 4-[3-(2, 6-dibenzyloxy-3-pyridyl)-1-methyl-indazol-7-yl]-3,6-dihydro-2H-pyridine-1-carboxylate

A mixture of 7-bromo-3-(2,6-dibenzyloxy-3-pyridyl)-1-methyl-indazole (3.0 g, 6.00 mmol, 1.0 equiv.), tert-butyl 4-(4,4,5,5-tetramethyl-1,3,2-dioxaborolan-2-yl)-3,6-dihydro-2H-pyridine-1-carboxylate (1.85 g, 6.00 mmol, 1.0 equiv.), and Ad₂nBuP Pd G₃ (436.63 mg, 599.54 μmol, 0.1 equiv.) and K₃PO₄ (3.82 g, 17.99 mmol, 3.0 equiv.) in dioxane (30 mL) was degassed and purged with N₂ three times, and then the mixture was stirred at 80° C. for 16 hours under N₂ atmosphere. The reaction was quenched by H₂O (120 mL) and extracted by ethyl acetate (150 mL×2). The organic phase was washed with brine (100 mL×2), dried with Na₂SO₄, and concentrated under reduced pressure to give a residue. The residue was purified by flash silica gel chromatography (eluent of 0˜30% ethyl acetate/petroleum ether) to give tert-butyl 4-[3-(2, 6-dibenzyloxy-3-pyridyl)-1-methyl-indazol-7-yl]-3,6-dihydro-2H-pyridine-1-carboxylate (2.4 g, 3.8 mmol, 63.6% yield, 95.0% purity) as yellow oil.

MS (ESI) m/z: 603.4 [M+H]⁺

¹H NMR (400 MHz, DMSO-d₆) δ=7.88 (d, J=8.4 Hz, 1H), 7.57 (dd, J=0.8, 8.4 Hz, 1H), 7.50-7.46 (m, 2H), 7.43-7.29 (m, 8H), 7.09 (dd, J=0.8, 6.8 Hz, 1H), 7.00 (d, J=8.4 Hz, 1H), 6.60 (d, J=8.4 Hz, 1H), 5.79 (s, 1H), 5.44 (d, J=4.8 Hz, 2H), 4.11-4.02 (m, 7H), 3.63 (t, J=5.2 Hz, 2H), 1.45 (s, 9H).

### Step B. Procedure for Preparation of tert-butyl 4-[3-(2, 6-dioxo-3-piperidyl)-1-methyl-indazol-7-yl] piperidine-1-carboxylate

A mixture of tert-butyl 4-[3-(2, 6-dibenzyloxy-3-pyridyl)-1-methyl-indazol-7-yl]-3,6-dihydro-2H-pyridine-1-carboxylate (2.4 g, 3.98 mmol, 1.0 equiv.), Pd/C (2 g, 10% purity, 1.0 equiv.), Pd(OH)₂ (2.0 g, 2.85 mmol, 20% purity) and AcOH (717.37 mg, 11.95 mmol, 683.21 μL, 3.0 equiv.) in THE (15 mL) and EtOH (15 mL) was degassed and purged with H₂ three times, and then the mixture was stirred at 25° C. for 16 hours under H₂ (15 Psi) atmosphere. The mixture solution was filtered and concentrated under reduced pressure to give tert-butyl 4-[3-(2, 6-dioxo-3-piperidyl)-1-methyl-indazol-7-yl] piperidine-1-carboxylate (1.6 g, crude) as a purple solid.

MS (ESI) m/z: 427.3 [M+H]⁺

### Step C. Procedure for Preparation of 3-[1-methyl-7-(4-piperidyl)indazol-3-yl]piperidine-2,6-dione

To a solution of tert-butyl 4-[3-(2, 6-dioxo-3-piperidyl)-1-methyl-indazol-7-yl] piperidine-1-carboxylate (1.6 g, 3.75 mmol, 1.0 equiv.) inn HCl/dioxane (10 mL). The mixture was stirred at 25° C. for 1 hour. The mixture was filtered to give 3-[1-methyl-7-(4-piperidyl)indazol-3-yl]piperidine-2,6-dione (1.1 g, crude) as a white solid.

### Step D. Procedure for Preparation of 6-[8-(1,3-benzothiazol-2-ylcarbamoyl)-3,4-dihydro-1H-isoquinolin-2-yl]-3-[2-methyl-3-[2-[1-(2-oxoethyl)-4-piperidyl]ethoxy]phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[2-[1-(2,2-diethoxyethyl)-4-piperidyl]ethoxy]-2-methyl-phenyl]pyridine-2-carboxylate (60 mg, 73.17 μmol, 1.0 equiv.) in HCOOH (1 mL) was stirred at 85 C for 1.5 hours. The reaction mixture was concentrated under reduced pressure to remove HCOOH. The residue was diluted with H₂O (10 mL) and extracted with ethyl acetate (10 mL×3). Then the mixture was dried, filtered, and concentrated under reduced pressure to give 6-[8-(1,3-benzothiazol-2-ylcarbamoyl)-3,4-dihydro-1H-isoquinolin-2-yl]-3-[2-methyl-3-[2-[1-(2-oxoethyl)-4-piperidyl]ethoxy]phenyl]pyridine-2-carboxylic acid (45 mg, crude) as a yellow oil and it was used into the next step without further purification.

MS (ESI) m/z: 708.4 [M+H₂O+H]⁺

### Step E. Procedure for Preparation of 6-[8-(1,3-benzothiazol-2-ylcarbamoyl)-3, 4-dihydro-1H-isoquinolin-2-yl]-3-[3-[2-[1-[2-[4-[3-(2,6-dioxo-3-piperidyl)-1-methyl-indazol-7-yl]-1-piperidyl]ethyl]-4-piperidyl]ethoxy]-2-methyl-phenyl] pyridine-2-carboxylic acid

A solution of 3-[1-methyl-7-(4-piperidyl)indazol-3-yl]piperidine-2,6-dione (14.19 mg, 43.49 μmol, 1.0 equiv.) and NaBH(OAc)₃ (27.65 mg, 130.47 μmol, 3.0 equiv.) in DCM (0.5 mL) and IPA (0.5 mL) was stirred at 0° C. 6-[8-(1,3-Benzothiazol-2-ylcarbamoyl)-3,4-dihydro-1H-isoquinolin-2-yl]-3-[2-methyl-3-[2-[1-(2-oxoethyl)-4-piperidyl]ethoxy]phenyl]pyridine-2-carboxylic acid (30 mg, 43.49 μmol, 1.0 equiv.) was added dropwise slowly at 0° C. After addition, the mixture was stirred at 0° C. for 1 hour, warmed to 25° C., and stirred for 2 hours. The reaction mixture was concentrated under reduced pressure to remove DCM (0.5 mL). The residue was diluted with H₂O (10 mL) and extracted with ethyl acetate (10 mL×3). The reaction mixture was then filtered, and the filtrate was concentrated under reduced pressure to give a residue. The residue was purified by prep-HPLC to give 6-[8-(1,3-benzothiazol-2-ylcarbamoyl)-3, 4-dihydro-1H-isoquinolin-2-yl]-3-[3-[2-[1-[2-[4-[3-(2,6-dioxo-3-piperidyl)-1-methyl-indazol-7-yl]-1-piperidyl]ethyl]-4-piperidyl]ethoxy]-2-methyl-phenyl] pyridine-2-carboxylic acid (7.3 mg, 7.2 μmol, 16.7% yield, 99.2% purity) as a yellow solid.

MS (ESI) m/z: 500.5 [M12+H]⁺

¹H NMR (400 MHz, DMSO-d₆) δ=10.92-10.86 (m, 1H), 8.02 (d, J=8.0 Hz, 1H), 7.78 (d, J=8.0 Hz, 1H), 7.63 (d, J=7.6 Hz, 1H), 7.53 (d, J=8.0 Hz, 1H), 7.50-7.28 (m, 3H), 7.39-7.32 (m, 2H), 7.24 (d, J=7.2 Hz, 1H), 7.10-7.05 (m, 2H), 6.94-6.87 (m, 2H), 6.66 (d, J=6.8 Hz, 1H), 4.98 (s, 2H), 4.39-4.33 (m, 1H), 4.20 (s, 2H), 4.02-3.97 (m, 3H), 3.91 (t, J=5.2 Hz, 2H), 3.08-3.01 (m, 6H), 2.98-2.85 (m, 2H), 2.75-2.61 (m, 5H), 2.23-2.14 (m, 3H), 2.09-1.95 (m, 3H), 1.91 (s, 3H), 1.87-1.81 (m, 2H), 1.79-1.76 (m, 1H), 1.75-1.60 (m, 5H), 1.55-1.41 (m, 1H), 1.29-1.21 (m, 2H)

### Example 196. Preparation of Compound 148b

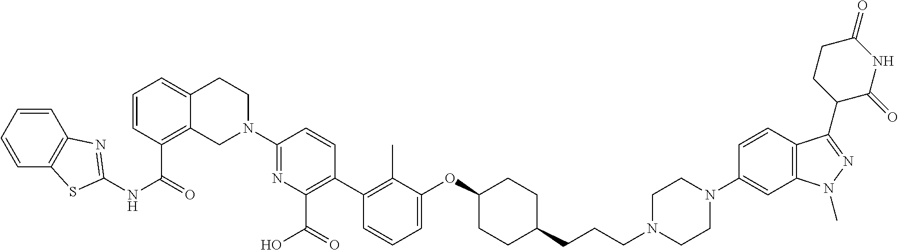 [SMILES: Cc1c(O[C@H]2CC[C@@H](CCCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4s)-4-(3-(4-(3-(2,6-dioxopiperidin-3-yl)-1-methyl-1H-indazol-6-yl)piperazin-1-yl)propyl)cyclohexyl)oxy)-2-methylphenyl)picolinic acid

### Step A. Procedure for Preparation of 3-[4-(3-bromo-2-methyl-phenoxy)cyclohexyl]propanal

A solution of DMSO (95.50 mg, 1.22 mmol, 95.50 μL, 4 equiv.) in DCM (5 mL) was added dropwise to a solution of oxalyl dichloride (77.57 mg, 611.14 μmol, 53.50 μL, 2 equiv.) in DCM (1 mL) at −70° C. under N₂ atmosphere. The mixture was stirred at −70° C. for 30 minutes. After which time 3-[4-(3-bromo-2-methyl-phenoxy)cyclohexyl]propan-1-ol (100 mg, 305.57 μmol, 1 equiv.) in DCM (1 mL) was added dropwise at −70° C. The solution was stirred for 30 minutes at −70° C. Then TEA (185.52 mg, 1.83 mmol, 255.19 μL, 6 equiv.) was added into the solution. The solution was stirred at 25° C. for 2 hour under N₂ atmosphere. The reaction mixture was quenched by addition H₂O (15 mL) at 25° C. and then extracted with DCM (20 mL×3). The combined organic layers were dried over Na₂SO₄, filtered, and concentrated under reduced pressure to give a residue. The residue was purified by flash silica gel chromatography (eluent of 0-10% ethyl acetate/petroleum ether) to give 3-[4-(3-bromo-2-methyl-phenoxy)cyclohexyl]propanal (100 mg, crude) as a yellow oil.

### Step B. Procedure for Preparation of tert-butyl 6-[8-(1,3-benzothiazol-2-ylcarbamoyl)-3,4-dihydro-1H-isoquinolin-2-yl]-3-[2-methyl-3-[4-(3-oxopropyl)cyclohexoxy]phenyl]pyridine-2-carboxylate

3-[4-(3-bromo-2-methyl-phenoxy)cyclohexyl]propanal (100 mg, 307.47 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (188.34 mg, 307.47 μmol, 1 equiv.), KF (53.59 mg, 922.40 mol, 21.61 μL, 3 equiv.), and Ad2nBuP Pd G3 (cataCXium® A Pd G3) (22.39 mg, 30.75 mol, 0.1 equiv.) were taken up into a microwave tube in dioxane (1 mL) and H₂O (0.1 mL). The sealed tube was heated at 100° C. for 60 min under microwave. The reaction mixture was concentrated under reduced pressure to give a residue. The residue was purified by flash silica gel chromatography (eluent of 0˜8% dichloromethane/methanol ether) to give tert-butyl 6-[8-(1,3-benzothiazol-2-ylcarbamoyl)-3,4-dihydro-1H-isoquinolin-2-yl]-3-[2-methyl-3-[4-(3-oxopropyl)cyclohexoxy]phenyl]pyridine-2-carboxylate (110 mg, 138.3 μmol, 44.9% yield, 91.9% purity) as a yellow oil.

MS (ESI) m/z: 731.6[M+H]⁺.

### Step C. Procedure for Preparation of tert-butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-6-yl]piperazin-1-yl]propyl]cyclohexoxy]-2-methyl-phenyl]pyridine-2-carboxylate

To a solution of tert-butyl 6-[8-(1,3-benzothiazol-2-ylcarbamoyl)-3,4-dihydro-1H-isoquinolin-2-yl]-3-[2-methyl-3-[4-(3-oxopropyl)cyclohexoxy]phenyl]pyridine-2-carboxylate (110 mg, 150.50 μmol, 1 equiv.) and 3-(1-methyl-6-piperazin-1-yl-indazol-3-yl)piperidine-2,6-dione (60.23 mg, 165.55 μmol, 1.1 equiv. HCl) in DCM (1.1 mL) was added AcOH (903.76 ug, 15.05 μmol, 0.861 μL, 0.1 equiv.). The mixture was stirred at 25° C. for 15.5 hours. NaBH(OAc)₃ (95.69 mg, 451.49 μmol, 3 equiv.) was then added. The mixture was stirred at 25° C. for 0.5 hour. The reaction mixture was concentrated under reduced pressure to give a residue. The residue was purified by flash silica gel chromatography (eluent of 0-10% dichloromethane/methanol) to give tert-butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-6-yl]piperazin-1-yl]propyl]cyclohexoxy]-2-methyl-phenyl]pyridine-2-carboxylate (110 mg, 77.5 μmol, 51.5% yield, 73.5% purity) as a yellow solid.

MS (ESI) m/z: 521.9[½ M+H]⁺.

### Step D. Procedure for Preparation of 6-(8-(benzo[d]thiazol-2-ylcarbamoyl)-3,4-dihydroisoquinolin-2(1H)-yl)-3-(3-(((1r,4s)-4-(3-(4-(3-(2,6-dioxopiperidin-3-yl)-1-methyl-1H-indazol-6-yl)piperazin-1-yl)propyl)cyclohexyl)oxy)-2-methylphenyl)picolinic acid

To a solution of tert-butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-6-yl]piperazin-1-yl]propyl]cyclohexoxy]-2-methyl-phenyl]pyridine-2-carboxylate (110 mg, 105.54 μmol, 1 equiv.) in TFA (0.6 mL) was added DCM (0.6 mL). The mixture was stirred at 25° C. for 16 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1r,4s)-4-(3-(4-(3-(2,6-dioxopiperidin-3-yl)-1-methyl-1H-indazol-6-yl)piperazin-1-yl)propyl)cyclohexyl)oxy)-2-methylphenyl)picolinic acid (23.2 mg, 23.0 μmol, 21.8% yield, 97.8% purity) as a grey solid.

MS (ESI) m/z: 986.4 [M+H]⁺.

1H NMR (400 MHz, DMSO-d₆) δ=10.85 (s, 1H), 8.15 (s, 1H), 8.07-7.98 (m, 1H), 7.78 (d, J=8.0 Hz, 1H), 7.65-7.59 (m, 1H), 7.51-7.42 (m, 4H), 7.40-7.31 (m, 2H), 7.07 (t, J=8.0 Hz, 1H), 6.98-6.90 (m, 2H), 6.90-6.87 (m, 1H), 6.83 (s, 1H), 6.60 (d, J=7.6 Hz, 1H), 4.97 (s, 2H), 4.61 (s, 1H), 4.25 (dd, J=5.2, 9.2 Hz, 1H), 3.93-3.86 (m, 5H), 3.21 (s, 4H), 3.02 (t, J=5.2 Hz, 2H), 2.62-2.58 (m, 2H), 2.54 (s, 4H), 2.35-2.31 (m, 2H), 2.20-2.12 (m, 1H), 1.93 (s, 5H), 1.61-1.46 (m, 6H), 1.40-1.17 (m, 6H)

### Example 197. Preparation of Compound 254a

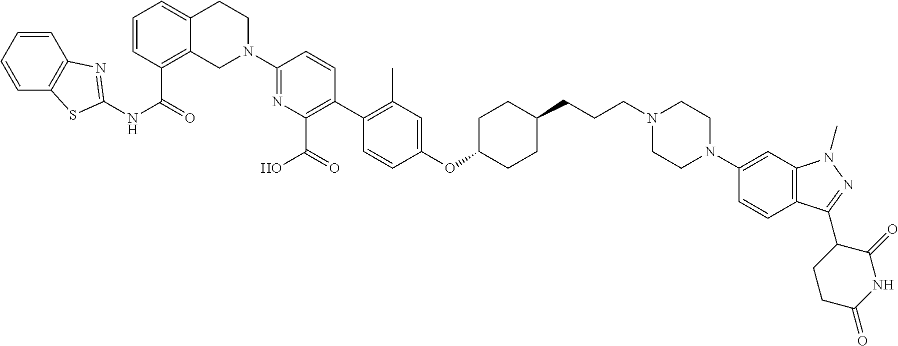 [SMILES: Cc1cc(O[C@H]2CC[C@H](CCCN3CCN(c4ccc5c(C6CCC(=O)NC6=O)nn(C)c5c4)CC3)CC2)ccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(4-(((1s,4r)-4-(3-(4-(3-(2,6-dioxopiperidin-3-yl)-1-methyl-1H-indazol-6-yl)piperazin-1-yl)propyl)cyclohexyl)oxy)-2-methylphenyl)picolinic acid

### Step A. Procedure for Preparation of (1r,4r)-methyl 4-(4-bromo-3-methylphenoxy)cyclohexanecarboxylate

To a solution of (1s,4s)-methyl 4-hydroxycyclohexanecarboxylate (2.0 g, 12.64 mmol, 1.1 equiv.) in toluene (20 mL) was added 2-(tributyl-λ5-phosphanylidene)acetonitrile (3.33 g, 13.79 mmol, 1.2 equiv.) and 4-bromo-3-methylphenol (2.15 g, 11.49 mmol, 1.0 equiv.). The mixture was stirred at 120° C. for 12 hours. The reaction mixture was concentrated under reduced pressure to remove solvent. The residue was purified by column chromatography (SiO₂, petroleum ether/ethyl acetate=1/0) to give (1r,4r)-methyl 4-(4-bromo-3-methylphenoxy)cyclohexanecarboxylate (2.4 g, crude) as a yellow oil.

### Step B. Procedure for Preparation of ((1r,4r)-4-(4-bromo-3-methylphenoxy)cyclohexyl)methanol

To a solution of (1r,4r)-methyl 4-(4-bromo-3-methylphenoxy)cyclohexanecarboxylate (2.4 g, 7.33 mmol, 1 equiv.) in THE (25 mL) was added LiAlH₄ (278.38 mg, 7.33 mmol, 1 equiv.). The mixture was stirred at 0° C. for 1 hour under N₂ atmosphere. The reaction mixture was quenched by addition H₂O (0.3 mL), 15% NaOH (0.3 mL), and H₂O (0.9 mL). The combined mixture was mixed with Na₂SO₄, and then filtered and concentrated under reduced pressure to give a residue. The residue was purified by flash silica gel chromatography (eluent of 0˜20% ethyl acetate/petroleum ether) to give compound ((1r,4r)-4-(4-bromo-3-methylphenoxy)cyclohexyl)methanol (1.3 g, 4.3 mmol, 59.2% yield) as a yellow oil.

¹H NMR (400 MHz) δ=7.40 (d, J=8.8 Hz, 1H), 6.94 (d, J=2.8 Hz, 1H), 6.75-6.67 (m, 1H), 4.43 (t, J=5.6 Hz, 1H), 4.26-4.15 (m, 1H), 3.23 (t, J=5.6 Hz, 2H), 2.28 (s, 3H), 2.08-2.00 (m, 2H), 1.77 (d, J=11.6 Hz, 2H), 1.43-1.33 (m, 1H), 1.33-1.22 (m, 2H), 1.11-0.98 (m, 2H)

### Step C. Procedure for Preparation of (1r,4r)-4-(4-bromo-3-methylphenoxy)cyclohexanecarbaldehyde

A solution of oxalyl dichloride (1.10 g, 8.69 mmol, 760.68 μL, 2 equiv.) in DCM (20 mL) was added dropwise to a solution of DMSO (1.36 g, 17.38 mmol, 1.36 mL, 4 equiv.) in DCM (2 mL) at −70° C. under N₂ atmosphere. The mixture was stirred at −70° C. for 1 hour. After which time ((1r,4r)-4-(4-bromo-3-methylphenoxy)cyclohexyl)methanol (1.3 g, 4.34 mmol, 1 equiv.) in DCM (2 mL) was added dropwise at −70° C. The solution was stirred for 1 hour at −70° C. Then TEA (2.64 g, 26.07 mmol, 3.63 mL, 6 equiv.) was added into the solution. The solution was stirred at −70° C. for 0.5 hour under N₂ atmosphere. DCM (50 mL) and water (50 mL) were added, and the layers were separated. The aqueous phase was washed with DCM (30 mL×2), dried over anhydrous sodium sulfate, filtered, and concentrated under vacuum to give (1r,4r)-4-(4-bromo-3-methylphenoxy)cyclohexanecarbaldehyde (1.2 g, 4.0 mmol, 92.9% yield) as a yellow oil.

### Step D. Procedure for Preparation of (E)-ethyl 3-((1r,4r)-4-(4-bromo-3-methylphenoxy)cyclohexyl)acrylate

To a solution of ethyl 2-(diethoxyphosphoryl)acetate (1.81 g, 8.08 mmol, 1.60 mL, 2.0 equiv.) in THE (15 mL) was added NaH (339.15 mg, 8.48 mmol, 60% purity, 2.1 equiv.). The mixture was stirred at 0° C. for 1 hour. Then (1r,4r)-4-(4-bromo-3-methylphenoxy)cyclohexanecarbaldehyde (1.2 g, 4.04 mmol, 1 equiv.) was added into the mixture and stirred at 25° C. for 0.5 hour under N₂. The reaction mixture was quenched by addition water (10 mL) at 0° C. and extracted with ethyl acetate (30 mL×3). The combined organic layers were washed with water (10 mL×3), filtered, and concentrated under reduced pressure to give a residue. The residue was purified by column chromatography (SiO₂, petroleum ether/ethyl acetate=0/1 to 10/1) to give (E)-ethyl 3-((1r,4r)-4-(4-bromo-3-methylphenoxy)cyclohexyl)acrylate (1 g, 2.7 mmol, 67.4% yield) as a white solid.

¹H NMR (400 MHz) δ=7.41 (d, J=8.8 Hz, 1H), 6.96 (d, J=2.8 Hz, 1H), 6.76-6.70 (m, 1H), 6.16-6.07 (m, 1H), 5.74 (d, J=11.6 Hz, 1H), 4.32-4.22 (m, 1H), 4.14-4.06 (m, 2H), 3.25-3.15 (m, 1H), 2.29 (s, 3H), 2.12-2.02 (m, 2H), 1.77-1.65 (m, 2H), 1.39-1.28 (m, 4H), 1.21 (t, J=7.2 Hz, 3H)

### Step E. Procedure for Preparation of ethyl 3-((1r,4r)-4-(4-bromo-3-methylphenoxy)cyclohexyl)propanoate

To a solution of (E)-ethyl 3-((1r,4r)-4-(4-bromo-3-methylphenoxy)cyclohexyl)acrylate (1 g, 2.72 mmol, 1 equiv.) in EtOH (10 mL) was added PtO₂ (61.83 mg, 272.27 μmol, 0.1 equiv.) under H₂ atmosphere (15 psi). The mixture was stirred at 25° C. for 12 hours. The reaction mixture was filtered and concentrated under reduced pressure to give a filter cake, rinsing with EtOH. The filter cake was dried under reduced pressure to give a residue. The residue was purified by column chromatography (SiO₂, petroleum ether/ethyl acetate=20/1 to 10/1) to give ethyl 3-((1r,4r)-4-(4-bromo-3-methylphenoxy)cyclohexyl)propanoate (700 mg, 1.7 mmol, 65.2% yield, 93.7% purity) as a colorless oil.

1H NMR (400 MHz) δ=7.40 (d, J=8.4 Hz, 1H), 6.94 (d, J=2.8 Hz, 1H), 6.74-6.67 (m, 1H), 4.28-4.16 (m, 1H), 4.09-4.00 (m, 2H), 2.34-2.22 (m, 5H), 2.02 (d, J=10.0 Hz, 2H), 1.74 (d, J=12.0 Hz, 2H), 1.51-1.40 (m, 2H), 1.34-1.22 (m, 3H), 1.21-1.15 (m, 3H), 1.11-0.98 (m, 2H)

### Step F. Procedure for Preparation of 3-((1r,4s)-4-(4-bromo-3-methylphenoxy)cyclohexyl)propan-1-ol

To a solution of ethyl 3-((1r,4r)-4-(4-bromo-3-methylphenoxy)cyclohexyl)propanoate (500 mg, 1.35 mmol, 1 equiv.) in THF (10 mL) was added LiAlH₄ (51.38 mg, 1.35 mmol, 1 equiv.) The mixture was stirred at 0° C. for 1 hour under N₂ atmosphere. The reaction mixture was quenched by addition of H₂O (0.1 mL), 15% NaOH (0.1 mL), and H₂O (0.3 mL). The combined mixture was mixed with Na₂SO₄, filtered, and concentrated under reduced pressure to give a residue. The residue was purified by flash silica gel chromatography (eluent of 0˜20% ethyl acetate/petroleum ether) to give 3-((1r,4s)-4-(4-bromo-3-methylphenoxy)cyclohexyl)propan-1-ol (400 mg, 1.2 mmol, 90.2% yield) as a yellow oil.

1H NMR (400 MHz,) 6=7.40 (d, J=8.8 Hz, 1H), 6.94 (d, J=2.8 Hz, 1H), 6.74-6.69 (m, 1H), 4.34 (t, J=5.2 Hz, 1H), 4.26-4.17 (m, 1H), 3.39-3.33 (m, 2H), 2.28 (s, 3H), 2.02 (d, J=10.0 Hz, 2H), 1.79-1.72 (m, 2H), 1.47-1.38 (m, 2H), 1.34-1.25 (m, 2H), 1.24-1.16 (m, 3H), 1.10-0.96 (m, 2H)

### Step G. Procedure for Preparation of 3-((1r,4r)-4-(4-bromo-3-methylphenoxy)cyclohexyl)propanal

A solution of oxalyl dichloride (310.29 mg, 2.44 mmol, 213.99 μL, 2.0 equiv.) in DCM (20 mL) was added dropwise to a solution of DMSO (381.99 mg, 4.89 mmol, 381.99 μL, 4.0 equiv.) in DCM (2 mL) at −70° C. under N₂ atmosphere. The mixture was stirred at −70° C. for 1 hour. After which time 3-((1r,4s)-4-(4-bromo-3-methylphenoxy)cyclohexyl)propan-1-ol (400 mg, 1.22 mmol, 1 equiv.) in DCM (2 mL) was added dropwise at −70° C. The solution was stirred for 1 hour at −70° C. Then TEA (742.10 mg, 7.33 mmol, 1.02 mL, 6 equiv.) was added into the solution. The solution was stirred at −70° C. for 0.5 hour under N₂ atmosphere. DCM (50 mL) and water (50 mL) were added, and layers were separated. The aqueous layer was extracted with DCM (30 mL×2). The combined organic extracts were dried over anhydrous sodium sulfate, filtered, and concentrated under vacuum to give 3-((1r,4r)-4-(4-bromo-3-methylphenoxy)cyclohexyl)propanal (270 mg, 830.1 μmol, 67.9% yield) as a yellow oil.

### Step H. Procedure for Preparation of tert-butyl 6-(8-(benzo[d]thiazol-2-ylcarbamoyl)-3,4-dihydroisoquinolin-2(1H)-yl)-3-(2-methyl-4-(((1r,4r)-4-(3-oxopropyl)cyclohexyl)oxy)phenyl)picolinate

To a solution of 3-((1r,4r)-4-(4-bromo-3-methylphenoxy)cyclohexyl)propanal (270 mg, 830.16 μmol, 1.0 equiv.) in dioxane (5 mL) was added KF (1.5 M, 1.66 mL, 3.0 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (508.51 mg, 830.16 μmol, 1.0 equiv.), and [2-(2-aminophenyl)phenyl]palladium(1⁺); bis(1-adamantyl)-butyl-phosphane; methanesulfonate (60.46 mg, 83.02 μmol, 0.1 equiv.). The mixture was stirred at 100° C. for 1 hour. The reaction mixture was concentrated under reduced pressure to remove solvent. The residue was purified by flash silica gel chromatography (eluent of 0˜50% ethyl acetate/petroleum ether) to give tert-butyl 6-(8-(benzo[d]thiazol-2-ylcarbamoyl)-3,4-dihydroisoquinolin-2(1H)-yl)-3-(2-methyl-4-(((1r,4r)-4-(3-oxopropyl)cyclohexyl)oxy)phenyl)picolinate (230 mg, 314.6 μmol, 37.9% yield) as a yellow solid.

MS (ESI) m/z: 731.5[M+H]⁺

### Step I. Procedure for preparation of tert-butyl 6-(8-(benzo[d]thiazol-2-ylcarbamoyl)-3,4-dihydroisoquinolin-2(1H)-yl)-3-(4-(((1s,4r)-4-(3-(4-(3-(2,6-dioxopiperidin-3-yl)-1-methyl-1H-indazol-6-yl)piperazin-1-yl)propyl)cyclohexyl)oxy)-2-methylphenyl)picolinate

To a solution of 3-(1-methyl-6-(piperazin-1-yl)-1H-indazol-3-yl)piperidine-2,6-dione (59.73 mg, 164.18 μmol, 1.2 equiv.) in DCM (0.5 mL) was added NaBH(OAc)₃ (86.99 mg, 410.45 μmol, 3.0 equiv.). Then tert-butyl 6-(8-(benzo[d]thiazol-2-ylcarbamoyl)-3,4-dihydroisoquinolin-2(1H)-yl)-3-(2-methyl-4-(((1r,4r)-4-(3-oxopropyl)cyclohexyl)oxy)phenyl)picolinate (100 mg, 136.82 μmol, 1.0 equiv.) was added into the mixture at 0° C. The mixture was stirred at 25° C. for 1 hour. The reaction mixture was concentrated under reduced pressure to give tert-butyl 6-(8-(benzo[d]thiazol-2-ylcarbamoyl)-3,4-dihydroisoquinolin-2(1H)-yl)-3-(4-(((1s,4r)-4-(3-(4-(3-(2,6-dioxopiperidin-3-yl)-1-methyl-1H-indazol-6-yl)piperazin-1-yl)propyl)cyclohexyl)oxy)-2-methylphenyl)picolinate (120 mg, 115.1 μmol, 84.1% yield) as a yellow solid.

MS (ESI) m/z: 1042.4 [M+H]⁺

### Step J. Procedure for Preparation of 6-(8-(benzo[d]thiazol-2-ylcarbamoyl)-3,4-dihydroisoquinolin-2(1H)-yl)-3-(4-(((1s,4r)-4-(3-(4-(3-(2,6-dioxopiperidin-3-yl)-1-methyl-1H-indazol-6-yl)piperazin-1-yl)propyl)cyclohexyl)oxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(4-(((1s,4r)-4-(3-(4-(3-(2,6-dioxopiperidin-3-yl)-1-methyl-1H-indazol-6-yl)piperazin-1-yl)propyl)cyclohexyl)oxy)-2-methylphenyl)picolinate (120 mg, 115.13 μmol, 1.0 equiv.) in DCM (1 mL) was added TFA (1.54 g, 13.51 mmol, 1 mL, 117.31 equiv.). The mixture was stirred at 25° C. for 12 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(4-(((1s,4r)-4-(3-(4-(3-(2,6-dioxopiperidin-3-yl)-1-methyl-1H-indazol-6-yl)piperazin-1-yl)propyl)cyclohexyl)oxy)-2-methylphenyl)picolinic acid (43.4 mg, 43.7 μmol, 37.9% yield, 99.2% purity) as a white solid.

MS (ESI) m/z: 986.4 [M+H]⁺

¹H NMR (400 MHz) δ=12.92-12.82 (m, 1H), 12.67-12.46 (m, 1H), 10.87 (s, 1H), 9.55-9.43 (m, 1H), 8.04 (d, J=8.0 Hz, 1H), 7.80 (d, J=8.0 Hz, 1H), 7.65-7.55 (m, 2H), 7.49-7.44 (m, 2H), 7.41-7.33 (m, 2H), 7.01-6.94 (m, 3H), 6.91 (d, J=8.4 Hz, 1H), 6.78 (d, J=2.3 Hz, 1H), 6.74-6.69 (m, 1H), 4.98 (s, 2H), 4.32-4.21 (m, 2H), 3.93 (s, 3H), 3.92-3.89 (m, 2H), 3.62 (d, J=10.4 Hz, 2H), 3.21-3.13 (m, 4H), 3.09-3.01 (m, 4H), 2.69-2.60 (m, 2H), 2.55-2.52 (m, 2H), 2.34-2.31 (m, 1H), 2.20-2.14 (m, 1H), 2.09 (d, J=11.6 Hz, 2H), 2.03 (s, 3H), 1.86-1.79 (m, 2H), 1.72 (d, J=4.0 Hz, 2H), 1.38-1.22 (m, 5H), 1.16-1.04 (m, 2H)

### Example 198. Preparation of Compound 269a

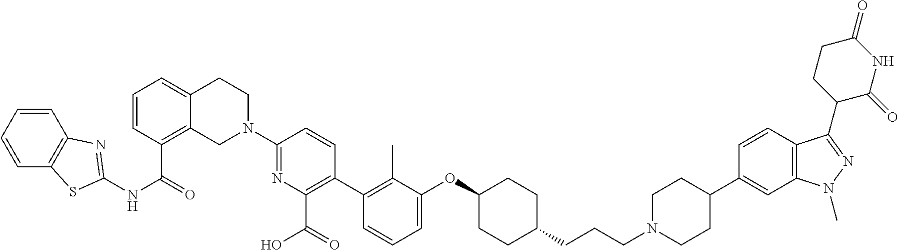 [SMILES: Cc1c(O[C@H]2CC[C@H](CCCN3CCC(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)piperidin-1-yl)propyl)cyclohexyl)oxy)-2-methylphenyl)picolinic acid

### Step A. Procedure for Preparation of 3-[4-(3-bromo-2-methyl-phenoxy)cyclohexyl]propan-1-01

To a solution of ethyl 3-[4-(3-bromo-2-methyl-phenoxy)cyclohexyl]propanoate (600 mg, 1.62 mmol, 1 equiv.) in THE (10 mL) was added LiAlH₄ (55.50 mg, 1.46 mmol, 0.9 equiv.) at 0° C. The mixture was stirred at 0° C. for 1.5 hours. The reaction mixture was quenched by addition H₂O (0.05 mL), 15% NaOH (0.05 mL), H₂O (15 mL), and then extracted with ethyl acetate 30 mL (10 mL×3). The combined organic layers were dried over Na₂SO₄, filtered, and concentrated under reduced pressure to give a residue. The residue was used in the next step without purification. The compound 3-[4-(3-bromo-2-methyl-phenoxy)cyclohexyl]propan-1-ol (500 mg, 1.5 mmol, 94.0% yield) was obtained as a white solid.

¹H NMR (400 MHz, DMSO-d₆) δ=7.14-7.02 (m, 3H), 4.23-4.21 (m, 1H), 3.37-3.36 (m, 3H), 3.34-3.35 (m, 1H), 2.26-2.24 (m, 1H), 2.21-2.19 (m, 2H), 2.06-2.04 (m, 2H), 1.76-1.72 (m, 2H), 1.41-1.42 (m, 2H), 1.40-1.39 (m, 2H), 1.38-1.36 (m, 2H), 1.20-1.15 (m, 3H), 1.03-0.99 (m, 2H).

### Step B. Procedure for Preparation of 3-[4-(3-bromo-2-methyl-phenoxy)cyclohexyl]propanal

A solution of (COCl)₂ (167.23 mg, 1.41 mmol, 101.97 μL, 2 equiv.) in DCM (30 mL) was added dropwise to a solution of DMSO (219.65 mg, 2.81 mmol, 219.65 μL, 4 equiv.) in DCM (30 mL) at −70° C. under N₂ atmosphere. The mixture was stirred at −70° C. for 1 hour. After which time 3-[4-(3-bromo-2-methyl-phenoxy)cyclohexyl]propan-1-ol (230 mg, 702.81 μmol, 1 equiv.) in DCM (30 mL) was added dropwise at −70° C. The solution was stirred for 1 hour at −70° C. Then TEA (426.70 mg, 4.22 mmol, 586.94 μL, 6 equiv.) was added into the solution. The solution was stirred at −70° C. for 1 hour under N₂ atmosphere. The reaction mixture was filtered and concentrated under reduced pressure to give a residue. The residue was purified by flash silica gel chromatography (eluent of 0˜43% ethyl acetate/petroleum ether gradient) to give 3-[4-(3-bromo-2-methyl-phenoxy)cyclohexyl]propanal (200 mg, 467.3 μmol, 66.5% yield, 76% purity) as a white solid.

### Step C. Procedure for Preparation of tert-butyl 6-[8-(1,3-benzothiazol-2-ylcarbamoyl)-3,4-dihydro-1H-isoquinolin-2-yl]-3-[2-methyl-3-[4-(3-oxopropyl)cyclohexoxy]phenyl]pyridine-2-carboxylate

A mixture of tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (414.34 mg, 676.42 μmol, 1.1 equiv.), 3-[4-(3-bromo-2-methyl-phenoxy)cyclohexyl]propanal (200 mg, 614.93 μmol, 1 equiv.), KF (1.5 M, 614.93 μL, 1.5 equiv.), and Ad₂nBuP Pd G₃ (cataCXium® A Pd G₃) (134.35 mg, 184.48 μmol, 0.3 equiv.) in 1,4-dioxane (15 mL) was degassed and purged with N₂ three times. The mixture was then stirred at 100° C. for 1 hour under microwave. The reaction mixture was filtered and concentrated under reduced pressure to give a residue. The residue was purified by flash silica gel chromatography (eluent of 0˜37% ethyl acetate/petroleum ether). The compound tert-butyl 6-[8-(1,3-benzothiazol-2-ylcarbamoyl)-3,4-dihydro-1H-isoquinolin-2-yl]-3-[2-methyl-3-[4-(3-oxopropyl)cyclohexoxy]phenyl]pyridine-2-carboxylate (300 mg, 410.4 μmol, 66.7% yield) was obtained as a yellow oil.

### Step D. Procedure for Preparation of tert-butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-6-yl]-1-piperidyl]propyl]cyclohexoxy]-2-methyl-phenyl]pyridine-2-carboxylate

A mixture of tert-butyl 6-[8-(1,3-benzothiazol-2-ylcarbamoyl)-3,4-dihydro-1H-isoquinolin-2-yl]-3-[2-methyl-3-[4-(3-oxopropyl)cyclohexoxy]phenyl]pyridine-2-carboxylate (50 mg, 68.41 μmol, 1 equiv.), and 3-[1-methyl-6-(4-piperidyl)indazol-3-yl]piperidine-2,6-dione (33.49 mg, 102.61 μmol, 1.5 equiv.) in DCM (1 mL) was degassed and purged with N₂ three times. The mixture was then stirred at 25° C. for 10 hours under N₂ atmosphere. NaBH(OAc)₃ (29.00 mg, 136.82 μmol, 2 equiv.) was then added to the mixture, which was stirred at 25° C. for 2 hours. The reaction mixture was filtered and concentrated under reduced pressure to give a residue. The compound tert-butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-6-yl]-1-piperidyl]propyl]cyclohexoxy]-2-methyl-phenyl]pyridine-2-carboxylate (60 mg, 57.6 μmol, 84.2% yield) was obtained as a white solid, which was used in the next step without further purification.

MS (ESI) m/z: 986.4 [M+H−56]⁺

### Step E. Procedure for Preparation of 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)piperidin-1-yl)propyl)cyclohexyl)oxy)-2-methylphenyl)picolinic acid

To a solution of tert-butyl 6-[8-(1,3-benzothiazol-2-ylcarbamoyl)-3,4-dihydro-1H-isoquinolin-2-yl]-3-[3-[4-[3-[4-[3-(2,6-dioxo-3-piperidyl)-1-methyl-indazol-6-yl]-1-piperidyl]propyl]cyclohexoxy]-2-methyl-phenyl]pyridine-2-carboxylate (50 mg, 48.02 μmol, 1 equiv.) in DCM (0.2 mL) was added TFA (769.98 mg, 6.75 mmol, 499.99 μL, 140.64 equiv.). The mixture was stirred at 25° C. for 0.5 hour. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)piperidin-1-yl)propyl)cyclohexyl)oxy)-2-methylphenyl)picolinic acid (16.6 mg, 16.2 μmol, 33.7% yield, 95.9% purity) as a brown solid.

MS (ESI) m/z: 986.4 [M+H−56]⁺

¹H NMR (400 MHz, DMSO-d₆) δ=10.91-10.74 (m, 1H), 8.30 (s, 2H), 7.99 (d, J=6.1 Hz, 1H), 7.76 (d, J=7.2 Hz, 1H), 7.63 (d, J=7.2 Hz, 1H), 7.49 (d, J=8.8 Hz, 1H), 7.45-7.38 (m, 2H), 7.34 (d, J=5.6 Hz, 2H), 7.07-6.98 (m, 1H), 6.91 (d, J=9.2 Hz, 1H), 6.85-6.81 (m, 2H), 6.69-6.67 (m, 1H), 5.04-4.91 (m, 2H), 4.26-4.24 (m, 1H), 3.91-3.89 (m, 5H), 3.22-3.20 (m, 4H), 3.02-3.00 (m, 4H), 2.64-2.58 (m, 2H), 2.35-2.29 (m, 4H), 2.21-2.12 (m, 2H), 2.09-2.07 (m, 3H), 1.89 (s, 3H), 1.82-1.80 (m, 2H), 1.49-1.47 (m, 2H), 1.39-1.33 (m, 2H), 1.27-1.16 (m, 4H), 1.10-1.03 (m, 2H).

### Example 199. Preparation of Compound 263b

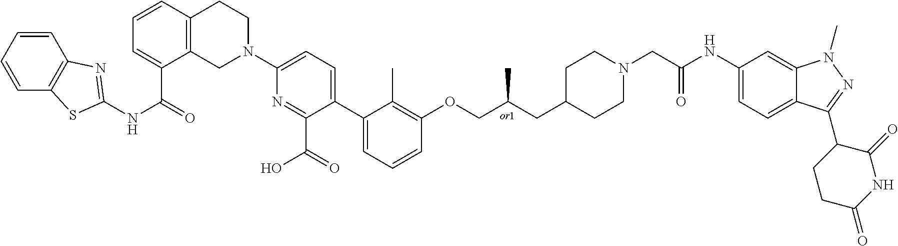 [SMILES: Cc1c(OC[C@@H](C)CC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[(2S)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylic acid

### [rel-6-(8-(benzo[d]thiazol-2-ylcarbamoyl)-3,4-dihydroisoquinolin-2(1H)-yl)-3-(3-((2S)-3-(1-(2-((3-(2,6-dioxopiperidin-3-yl)-1-methyl-1H-indazol-6-yl)amino)-2-oxoethyl)piperidin-4-yl)-2-methylpropoxy)-2-methylphenyl)picolinic acid]

### Step A. Procedure for Preparation of ethyl 2-[4-[(2S)-3-(3-bromo-2-methyl-phenoxy)-2-methyl-propyl]-1-piperidyl]acetate

The stereoisomers of ethyl 2-[4-[3-(3-bromo-2-methyl-phenoxy)-2-methyl-propyl]-1-piperidyl]acetate (1 g, 2.43 mmol, 1.00 equiv.) were separated by SFC to give ethyl 2-[4-[(2S)-3-(3-bromo-2-methyl-phenoxy)-2-methyl-propyl]-1-piperidyl]acetate (0.3 g, 727.5 μmol, 30.0% yield) as a colorless oil.

### Step B. Procedure for Preparation of tert-butyl 6-[8-(1,3-benzothiazol-2-ylcarbamoyl)-3,4-dihydro-1H-isoquinolin-2-yl]-3-[3-[(2S)-3-[1-(2-ethoxy-2-oxo-ethyl)-4-piperidyl]-2-methyl-propoxy]-2-methyl-phenyl]pyridine-2-carboxylate

A mixture of ethyl 2-[4-[(2S)-3-(3-bromo-2-methyl-phenoxy)-2-methyl-propyl]-1-piperidyl]acetate (150.00 mg, 363.76 μmol, 1.00 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (222.82 mg, 363.76 μmol, 1.00 equiv.), Ad₂nBuP Pd G₃ (26.49 mg, 36.38 μmol, 0.10 equiv.), and K₃PO₄ (231.64 mg, 1.09 mmol, 3.00 equiv.) in dioxane (2 mL) and H₂O (0.2 mL) was degassed and purged with N₂ three times. The mixture was then stirred at 80° C. for 16 hours under N₂ atmosphere. The reaction mixture was concentrated under reduced pressure to give a residue. The residue was purified by prep-TLC (SiO₂, petroleum ether/ethyl acetate=1:1) to give tert-butyl 6-[8-(1,3-benzothiazol-2-ylcarbamoyl)-3,4-dihydro-1H-isoquinolin-2-yl]-3-[3-[(2S)-3-[1-(2-ethoxy-2-oxo-ethyl)-4-piperidyl]-2-methyl-propoxy]-2-methyl-phenyl]pyridine-2-carboxylate (140 mg, 171.1 μmol, 47.0% yield) was obtained as a yellow solid.

MS (ESI) m/z: 409.9 [M/2+H]⁺.

### Step C. Procedure for Preparation of 2-[4-[(2S)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-2-methyl-propyl]-1-piperidyl]acetic acid

To a solution of tert-butyl 6-[8-(1,3-benzothiazol-2-ylcarbamoyl)-3,4-dihydro-1H-isoquinolin-2-yl]-3-[3-[(2S)-3-[1-(2-ethoxy-2-oxo-ethyl)-4-piperidyl]-2-methyl-propoxy]-2-methyl-phenyl]pyridine-2-carboxylate (140 mg, 171.14 μmol, 1.00 equiv.) in THE (2 mL) and H₂O (0.5 mL) was added LiOH·H₂O (21.55 mg, 513.43 μmol, 3.00 equiv.). The mixture was stirred at 25° C. for 3 minutes. The reaction mixture was concentrated under reduced pressure to give crude 2-[4-[(2S)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-2-methyl-propyl]-1-piperidyl]acetic acid (100 mg, 126.5 μmol, 73.9% yield) as a yellow solid.

MS (ESI) m/z 790.4 [M+H]⁺.

### Step D. Procedure for Preparation of tert-butyl 6-[8-(1,3-benzothiazol-2-ylcarbamoyl)-3,4-dihydro-1H-isoquinolin-2-yl]-3-[3-[(2S)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylate

To a solution of 2-[4-[(2S)-3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]-2-methyl-propyl]-1-piperidyl]acetic acid (80 mg, 101.27 μmol, 1.00 equiv.) 3-(6-amino-1-methyl-indazol-3-yl)piperidine-2,6-dione (28.77 mg, 111.40 μmol, 1.10 equiv.) in DMF (1 mL) was added HATU (46.21 mg, 121.52 μmol, 1.20 equiv.) and TEA (30.74 mg, 303.81 μmol, 42.29 μL, 3.00 equiv.). The mixture was stirred at 40° C. for 16 hours. The solution was then poured into water (2 mL) and filtered to give tert-butyl 6-[8-(1,3-benzothiazol-2-ylcarbamoyl)-3,4-dihydro-1H-isoquinolin-2-yl]-3-[3-[(2S)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylate (80 mg, 77.6 μmol, 76.6% yield) as a pink solid.

MS (ESI) m/z: 516.0 [M12+H]⁺.

### Step E. Procedure for Preparation of 6-[8-(1,3-benzothiazol-2-ylcarbamoyl)-3,4-dihydro-1H-isoquinolin-2-yl]-3-[3-[(2S)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylic acid [rel-6-(8-(benzo[d]thiazol-2-ylcarbamoyl)-3,4-dihydroisoquinolin-2(1H)-yl)-3-(3-((2S)-3-(1-(2-((3-(2,6-dioxopiperidin-3-yl)-1-methyl-1H-indazol-6-yl)amino)-2-oxoethyl)piperidin-4-yl)-2-methylpropoxy)-2-methylphenyl)picolinic acid]

To a solution of tert-butyl 6-[8-(1,3-benzothiazol-2-ylcarbamoyl)-3,4-dihydro-1H-isoquinolin-2-yl]-3-[3-[(2S)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylate (80 mg, 77.65 μmol, 1.00 equiv.) in DCM (1 mL) was added TFA (616.00 mg, 5.40 mmol, 400.00 μL, 69.57 equiv.). The mixture was stirred at 40° C. for 16 hours. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[(2S)-3-[1-[2-[[3-(2,6-dioxo-3-piperidyl)-1-methyl-indazol-6-yl]amino]-2-oxo-ethyl]-4-piperidyl]-2-methyl-propoxy]-2-methyl-phenyl]pyridine-2-carboxylic acid [rel-6-(8-(benzo[d]thiazol-2-ylcarbamoyl)-3,4-dihydroisoquinolin-2(1H)-yl)-3-(3-((2S)-3-(1-(2-((3-(2,6-dioxopiperidin-3-yl)-1-methyl-1H-indazol-6-yl)amino)-2-oxoethyl)piperidin-4-yl)-2-methylpropoxy)-2-methylphenyl)picolinic acid] (18.8 mg, 17.8 μmol, 22.9% yield, 96.4% purity) as a yellow solid

MS (ESI) m/z: 488.0 [M12+H]⁺

¹H NMR (400 MHz, DMSO-d₆) δ=10.88 (s, 1H), 9.90-9.75 (m, 1H), 8.19 (s, 1H), 8.08-7.97 (m, 2H), 7.78 (d, J=8.0 Hz, 1H), 7.62 (d, J=8.4 Hz, 2H), 7.48-7.40 (m, 3H), 7.39-7.31 (m, 2H), 7.21 (dd, J=1.6, 8.8 Hz, 1H), 7.11-7.04 (m, 1H), 6.92 (d, J=8.4 Hz, 1H), 6.86 (d, J=8.4 Hz, 1H), 6.64 (d, J=7.5 Hz, 1H), 5.01-4.94 (m, 2H), 4.32 (dd, J=5.2, 9.6 Hz, 1H), 3.93-3.88 (m, 5H), 3.83-3.74 (m, 2H), 3.12 (s, 2H), 3.02 (t, J=5.2 Hz, 2H), 2.90-2.83 (m, 2H), 2.66-2.60 (m, 2H), 2.19-2.14 (m, 2H), 2.03 (s, 2H), 1.94-1.89 (m, 3H), 1.71-1.63 (m, 2H), 1.49-1.39 (m, 2H), 1.32-1.13 (m, 4H), 1.03-0.98 (m, 3H)

### Example 200. Preparation of Compound 264

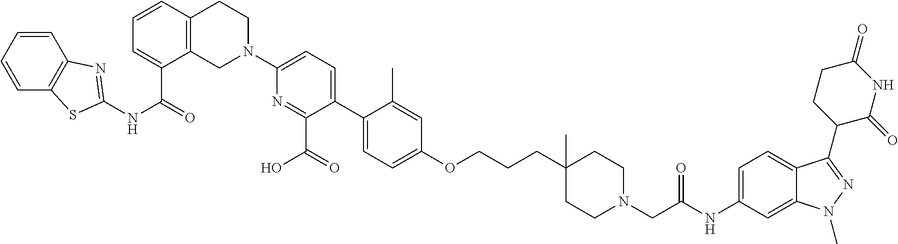 [SMILES: Cc1cc(OCCCC2(C)CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[4-[3-[1-[2-[[3-(2,6-dioxo-3-piperidyl)-1-methyl-indazol-6-yl]amino]-2-oxo-ethyl]-4-methyl-4-piperidyl]propoxy]-2-methyl-phenyl]pyridine-2-carboxylic acid

### Step A. Procedure for Preparation of tert-butyl 4-[3-(4-bromo-3-methyl-phenoxy)propyl]-4-methyl-piperidine-1-carboxylate

To a solution of tert-butyl 4-(3-hydroxypropyl)-4-methyl-piperidine-1-carboxylate (250 mg, 971.37 μmol, 1.0 equiv.) in toluene (3 mL) and 4-bromo-3-methyl-phenol (218.01 mg, 1.17 mmol, 1.2 equiv.) was added 2-(tributyl-λ5-phosphanylidene)acetonitrile (351.67 mg, 1.46 mmol, 1.5 equiv.). The mixture was stirred at 120° C. for 12 hours. The reaction mixture was concentrated under reduced pressure to give a residue. The residue was purified by column chromatography (SiO₂, petroleum ether/ethyl acetate=I/O to 10/1) to give tert-butyl 4-[3-(4-bromo-3-methyl-phenoxy)propyl]-4-methyl-piperidine-1-carboxylate (300 mg, 703.5 μmol, 72.4% yield) as a yellow oil.

### Step B. Procedure for Preparation of 4-[3-(4-bromo-3-methyl-phenoxy)propyl]-4-methyl-piperidine

A solution of tert-butyl 4-[3-(4-bromo-3-methyl-phenoxy)propyl]-4-methyl-piperidine-1-carboxylate (300 mg, 703.59 μmol, 1.0 equiv.) in HCl/dioxane (3 mL) was stirred at 25° C. for 1 hour. The reaction mixture was concentrated under reduced pressure to give 4-[3-(4-bromo-3-methyl-phenoxy)propyl]-4-methyl-piperidine (180 mg, 551.6 μmol, 78.4% yield) as a white solid.

### Step C. Procedure for Preparation of ethyl 2-[4-[3-(4-bromo-3-methyl-phenoxy)propyl]-4-methyl-1-piperidyl]acetate

To a solution of 4-[3-(4-bromo-3-methyl-phenoxy)propyl]-4-methyl-piperidine (180 mg, 551.69 μmol, 1.0 equiv.) and ethyl 2-bromoacetate (92.13 mg, 551.69 μmol, 61.01 μL, 1.0 equiv.) in CH₃CN (2 mL) was added K₂CO₃ (228.74 mg, 1.66 mmol, 3.0 equiv.). The mixture was stirred at 60° C. for 1 hour. The reaction mixture was filtered and concentrated under reduced pressure to give ethyl 2-[4-[3-(4-bromo-3-methyl-phenoxy)propyl]-4-methyl-1-piperidyl]acetate (190 mg, 460.7 μmol, 83.5% yield) as a yellow oil.

MS (ESI) m/z: 412.0 [M+H]⁺

¹H NMR (400 MHz, DMSO-d₆) δ=7.42 (d, J=8.4 Hz, 1H), 6.94 (d, J=2.8 Hz, 1H), 6.74-6.68 (m, 1H), 4.11-4.04 (m, 2H), 3.92 (t, J=6.4 Hz, 2H), 3.18 (s, 2H), 2.46-2.37 (m, 3H), 2.29 (s, 3H), 1.69-1.58 (m, 2H), 1.41-1.25 (m, 7H), 1.20-1.16 (m, 3H), 0.86 (s, 3H).

### Step D. Procedure for Preparation of tert-butyl 6-[8-(1,3-benzothiazol-2-ylcarbamoyl)-3,4-dihydro-1H-isoquinolin-2-yl]-3-[4-[3-[1-(2-ethoxy-2-oxo-ethyl)-4-methyl-4-piperidyl]propoxy]-2-methyl-phenyl]pyridine-2-carboxylate

Ethyl 2-[4-[3-(4-bromo-3-methyl-phenoxy)propyl]-4-methyl-1-piperidyl]acetate (190 mg, 460.76 μmol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (338.69 mg, 552.92 μmol, 1.2 equiv.), [2-(2-aminophenyl)phenyl]palladium(1+); bis(1-adamantyl)-butyl-phosphane; methanesulfonate (33.56 mg, 46.08 μmol, 0.1 equiv.), and KF (1.5 M, 921.53 μL, 3.0 equiv.) were taken up into a microwave tube in dioxane (3 mL). The sealed tube was heated at 100° C. for 1 hour under microwave. The reaction mixture was filtered and concentrated under reduced pressure to give a residue. The residue was purified by column chromatography (SiO₂, petroleum ether/ethyl acetate=10/1 to 1/1) to give tert-butyl 6-[8-(1,3-benzothiazol-2-ylcarbamoyl)-3,4-dihydro-1H-isoquinolin-2-yl]-3-[4-[3-[1-(2-ethoxy-2-oxo-ethyl)-4-methyl-4-piperidyl]propoxy]-2-methyl-phenyl]pyridine-2-carboxylate (230 mg, 281.1 μmol, 61.0% yield) as a yellow solid.

MS (ESI) m/z: 818.4 [M+H]⁺

### Step E. Procedure for Preparation of 2-[4-[3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]propyl]-4-methyl-1-piperidyl]acetic acid

To a solution of tert-butyl 6-[8-(1,3-benzothiazol-2-ylcarbamoyl)-3,4-dihydro-1H-isoquinolin-2-yl]-3-[4-[3-[1-(2-ethoxy-2-oxo-ethyl)-4-methyl-4-piperidyl]propoxy]-2-methyl-phenyl]pyridine-2-carboxylate (230 mg, 281.16 μmol, 1.0 equiv.) in THE (1 mL) and H₂O (1 mL) was added LiOH·H₂O (67.34 mg, 2.81 mmol, 10.0 equiv.). The mixture was stirred at 25° C. for 1 hour. The reaction mixture was adjusted pH to 3-4 with HCl (1 M), and extracted with DCM (2 mL×3). The combined organic layers were concentrated under reduced pressure to give 2-[4-[3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]propyl]-4-methyl-1-piperidyl]acetic acid (200 mg, 253.1 μmol, 90.0% yield) as a white solid.

### Step F. Procedure for Preparation of tert-butyl 6-[8-(1,3-benzothiazol-2-ylcarbamoyl)-3,4-dihydro-1H-isoquinolin-2-yl]-3-[4-[3-[1-[2-[[3-(2,6-dioxo-3-piperidyl)-1-methyl-indazol-6-yl]amino]-2-oxo-ethyl]-4-methyl-4-piperidyl]propoxy]-2-methyl-phenyl]pyridine-2-carboxylate

To a solution of 2-[4-[3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]propyl]-4-methyl-1-piperidyl]acetic acid (100 mg, 126.59 μmol, 1.0 equiv.) and 3-(6-amino-1-methyl-indazol-3-yl)piperidine-2,6-dione (39.23 mg, 151.90 μmol, 1.2 equiv.) in pyridine (1 mL) was added EDCI (36.40 mg, 189.88 μmol, 1.5 equiv.). The mixture was stirred at 25° C. for 1 hour. The reaction mixture was quenched by adding water (5 mL), and then filtered and concentrated under reduced pressure to give tert-butyl 6-[8-(1,3-benzothiazol-2-ylcarbamoyl)-3,4-dihydro-1H-isoquinolin-2-yl]-3-[4-[3-[1-[2-[[3-(2,6-dioxo-3-piperidyl)-1-methyl-indazol-6-yl]amino]-2-oxo-ethyl]-4-methyl-4-piperidyl]propoxy]-2-methyl-phenyl]pyridine-2-carboxylate (100 mg, 97.0 μmol, 76.6% yield) as a yellow solid.

MS (ESI) m/z: 516.0 [M12+H]⁺

### Step G. Procedure for Preparation of 6-[8-(1,3-benzothiazol-2-ylcarbamoyl)-3,4-dihydro-1H-isoquinolin-2-yl]-3-[4-[3-[1-[2-[[3-(2,6-dioxo-3-piperidyl)-1-methyl-indazol-6-yl]amino]-2-oxo-ethyl]-4-methyl-4-piperidyl]propoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[4-[3-[1-[2-[[3-(2,6-dioxo-3-piperidyl)-1-methyl-indazol-6-yl]amino]-2-oxo-ethyl]-4-methyl-4-piperidyl]propoxy]-2-methyl-phenyl]pyridine-2-carboxylate (100 mg, 97.06 μmol, 1.0 equiv.) in DCM (1 mL) and TFA (1 mL). The mixture was stirred at 25° C. for 12 hours. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[4-[3-[1-[2-[[3-(2,6-dioxo-3-piperidyl)-1-methyl-indazol-6-yl]amino]-2-oxo-ethyl]-4-methyl-4-piperidyl]propoxy]-2-methyl-phenyl]pyridine-2-carboxylic acid (37.8 mg, 37.4 μmol, 38.6% yield, 96.6% purity) as a white solid.

MS (ESI) m/z: 974.4 [M+H]⁺

¹H NMR (400 MHz, DMSO-d₆) δ=10.86 (s, 1H), 9.82 (s, 1H), 8.17 (s, 1H), 8.07-7.99 (m, 2H), 7.78 (d, J=8.0 Hz, 1H), 7.63 (d, J=8.8 Hz, 2H), 7.49-7.40 (m, 3H), 7.39-7.31 (m, 2H), 7.25-7.19 (m, 1H), 6.92 (d, J=8.4 Hz, 2H), 6.79 (d, J=1.6 Hz, 1H), 6.74-6.66 (m, 1H), 4.97 (s, 2H), 4.37-4.27 (m, 1H), 3.97-3.88 (m, 7H), 3.17-3.15 (m, 2H), 3.05-3.00 (m, 2H), 2.66-2.55 (m, 6H), 2.35-2.31 (m, 1H), 2.21-2.14 (m, 1H), 2.03 (s, 3H), 1.74-1.64 (m, 2H), 1.55-1.46 (m, 2H), 1.44-1.35 (m, 4H), 0.92 (s, 3H).

### Example 201. Preparation of Compound 265

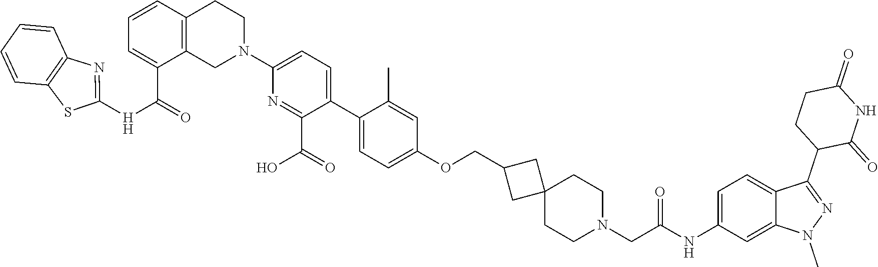

### 6-(8-(benzo[d]thiazol-2-ylcarbamoyl)-3, 4-dihydroisoquinolin-2(1H)-yl)-3-(4-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-methylphenyl)picolinic acid

### Step A. Procedure for Preparation of tert-butyl 2-((4-bromo-3-methylphenoxy)methyl)-7-azaspiro[3.5]nonane-7-carboxylate

A mixture of tert-butyl 2-(hydroxymethyl)-7-azaspiro[3.5]nonane-7-carboxylate (600 mg, 2.35 mmol, 1 equiv.), 4-bromo-3-methyl-phenol (571.31 mg, 3.06 mmol, 1.3 equiv.), and 2-(tributyl-λ⁵-phosphanylidene)acetonitrile (850.66 mg, 3.53 mmol, 1.5 equiv.) in toluene (12 mL) was degassed and purged with N₂ three times. The mixture was then stirred at 120° C. for 5 hours under N₂ atmosphere. The reaction mixture was diluted with water (10 mL) and extracted with EtOAc (25 mL×3). The combined organic layers were washed with brine (25 mL×3), dried over anhydrous sodium sulfate, filtered, and concentrated under reduced pressure to give a residue. The residue was purified by prep-TLC (SiO₂, petroleum ether:ethyl acetate=10:1) to give tert-butyl 2-((4-bromo-3-methylphenoxy)methyl)-7-azaspiro[3.5]nonane-7-carboxylate (693 mg, 1.6 mmol, 69.4% yield) as yellow oil.

### Step B. Procedure for Preparation of 2-[(4-bromo-3-methyl-phenoxy)methyl]-7-azaspiro[3.5]nonane

A mixture of tert-butyl 2-((4-bromo-3-methylphenoxy)methyl)-7-azaspiro[3.5]nonane-7-carboxylate (693 mg, 1.63 mmol, 1 equiv.) in HCl/dioxane (6 mL) and DCM (6 mL) was degassed and purged with N₂ three times. The mixture was then stirred at 25° C. for 16 hours under N₂ atmosphere. The reaction mixture was concentrated under reduced pressure to give 2-[(4-bromo-3-methyl-phenoxy)methyl]-7-azaspiro[3.5]nonane (1.54 g, crude) as white solid.

¹HNMR (400 MHz, CDCl₃) δ=9.42-9.36 (m, 1H), 7.40 (d, J=8.4 Hz, 1H), 6.78 (d, J=2.8 Hz, 1H), 6.59 (dd, J=8.4, 2.8 Hz, 1H), 3.88 (d, J=5.6 Hz, 2H), 3.18-3.12 (m, 2H), 3.12-3.05 (m, 2H), 2.76-2.69 (m, 1H), 2.36 (s, 3H), 2.09-2.02 (m, 2H), 2.02-1.99 (m, 2H), 1.94-1.88 (m, 2H), 1.81-1.75 (m, 2H)

### Step C. Procedure for Preparation of ethyl 2-[2-[(4-bromo-3-methyl-phenoxy)methyl]-7-azaspiro[3.5]nonan-7-yl]acetate

A mixture of 2-[(4-bromo-3-methyl-phenoxy)methyl]-7-azaspiro[3.5]nonane (1.54 g, 4.27 mmol, 1 equiv., HCl), ethyl 2-bromoacetate (712.97 mg, 4.27 mmol, 472.17 μL, 1 equiv.), and K₂CO₃ (1.77 g, 12.81 mmol, 3 equiv.) in DMF (23 mL) was degassed and purged with N₂ three times. The mixture was then stirred at 60° C. for 2 hours under N₂ atmosphere. The reaction mixture was diluted with water (25 mL) and extracted with EtOAc (25 mL×3). The combined organic layers were washed with brine (25 mL) and dried over anhydrous sodium sulfate, filtered, and concentrated under reduced pressure to give a residue. The residue was purified by prep-TLC (SiO₂, petroleum ether: ethyl acetate=10:1) to give ethyl 2-[2-[(4-bromo-3-methyl-phenoxy)methyl]-7-azaspiro[3.5]nonan-7-yl]acetate (252 mg, 614.1 μmol, 14.3% yield) as a colorless oil.

MS (ESI) m/z: 410.2 [M+H]⁺

¹HNMR (400 MHz, CDCl₃) δ=7.39 (d, J=8.8 Hz, 1H), 6.78 (d, J=2.8 Hz, 1H), 6.60 (dd, J=8.8, 3.2 Hz, 1H), 4.25-4.15 (m, 2H), 3.88 (d, J=6.4 Hz, 2H), 3.58-3.41 (m, 1H), 3.35-3.10 (m, 2H), 2.76-2.61 (m, 2H), 2.36 (s, 3H), 2.01-1.95 (m, 2H), 1.83-1.74 (m, 2H), 1.74-1.67 (m, 2H), 1.67-1.64 (m, 2H), 1.64-1.60 (m, 2H), 1.36-1.34 (m, 1H), 1.35 (s, 1H), 1.29 (t, J=7.2 Hz, 3H)

### Step D. Procedure for Preparation of tert-butyl 6-(8-(benzo[d]thiazol-2-ylcarbamoyl)-3,4-dihydroisoquinolin-2(1H)-yl)-3-(4-((7-(2-ethoxy-2-oxoethyl)-7-azaspiro[3.5]nonan-2-yl)methoxy)-2-methylphenyl)picolinate

Ethyl 2-[2-[(4-bromo-3-methyl-phenoxy)methyl]-7-azaspiro[3.5]nonan-7-yl]acetate (100 mg, 243.70 μmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (149.28 mg, 243.70 μmol, 1 equiv.), Ad₂nBuP Pd G₃ (cataCXium® A Pd G₃) (17.75 mg, 24.37 μmol, 0.1 equiv.), and KF (1.5 M, 487.40 μL, 3 equiv.) were taken up into a microwave tube in dioxane (1 mL). The sealed tube was heated at 100° C. for 1 hour under microwave. The reaction mixture was diluted with water (25 mL) and extracted with EtOAc (25 mL×3). The combined organic layers were washed with brine (25 mL), dried over anhydrous sodium sulfate, filtered, and concentrated under reduced pressure to give a residue. The residue was purified by prep-TLC (SiO₂, petroleum ether: ethyl acetate=1:1) to give tert-butyl 6-(8-(benzo[d]thiazol-2-ylcarbamoyl)-3,4-dihydroisoquinolin-2(1H)-yl)-3-(4-((7-(2-ethoxy-2-oxoethyl)-7-azaspiro[3.5]nonan-2-yl)methoxy)-2-methylphenyl)picolinate (147 mg, 180.1 μmol, 73.9% yield) as a light yellow solid.

MS (ESI) m/z: 816.3 [M+H]⁺

¹HNMR (400 MHz, CDCl₃) δ=7.86 (d, J=7.6 Hz, 1H), 7.76 (d, J=8.4 Hz, 1H), 7.58 (d, J=11.2 Hz, 1H), 7.48-7.42 (m, 1H), 7.40 (d, J=8.8 Hz, 1H), 7.36 (d, J=5.2 Hz, 1H), 7.35 (s, 1H), 7.30 (d, J=6.0 Hz, 1H), 7.00-6.96 (m, 1H), 6.88 (d, J=8.8 Hz, 1H), 6.77 (d, J=2.8 Hz, 1H), 6.71-6.67 (m, 1H), 5.07-4.98 (m, 2H), 4.28-4.14 (m, 2H), 4.11-4.08 (m, 2H), 3.93 (d, J=6 Hz, 1H), 3.10-3.05 (m, 2H), 2.78-2.69 (m, 1H), 2.10 (s, 3H), 2.02-1.94 (m, 1H), 1.94-1.84 (m, 2H), 1.76 (d, J=3.6 Hz, 2H), 1.74-1.68 (m, 2H), 1.32-1.29 (m, 1H), 1.32-1.29 (m, 1H), 1.28-1.26 (m, 2H), 1.25 (s, 2H), 1.19 (s, 9H), 1.09 (s, 2H)

### Step E. Procedure for Preparation of 2-[2-[[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]methyl]-7-azaspiro[3.5]nonan-7-yl] acetic acid

A mixture of tert-butyl 6-(8-(benzo[d]thiazol-2-ylcarbamoyl)-3,4-dihydroisoquinolin-2(1H)-yl)-3-(4-((7-(2-ethoxy-2-oxoethyl)-7-azaspiro[3.5]nonan-2-yl)methoxy)-2-methylphenyl)picolinate (147 mg, 180.14 μmol, 1 equiv.) and LiOH·H₂O (37.80 mg, 900.72 μmol, 5 equiv.) in THF (1.5 mL) and H₂O (1.5 mL) was degassed and purged with N₂ three times. The mixture was then stirred at 25° C. for 16 hours under N₂ atmosphere. The mixture was concentrated under reduced pressure to give a residue. To this residue, HCl (1 M, 1 mL) was slowly added dropwise to until pH=3-4, when a yellow solid was precipitated and collected by filtration. The yellow solid was dissolved in DCM (10 mL) and concentrated under reduced pressure to give 2-[2-[[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]methyl]-7-azaspiro[3.5]nonan-7-yl] acetic acid (101 mg, crude).

¹HNMR (400 MHz, DMSO) δ=13.18-12.53 (m, 1H), 8.01 (d, J=8.0 Hz, 1H), 7.77 (d, J=8.0 Hz, 1H), 7.59 (d, J=7.6 Hz, 1H), 7.49-7.45 (m, 1H), 7.43 (d, J=2.8 Hz, 1H), 7.39 (d, J=7.2 Hz, 1H), 7.37-7.34 (m, 1H), 7.34-7.31 (m, 1H), 6.97-6.90 (m, 1H), 6.856 (d, J=8.4 Hz, 1H), 6.81 (d, J=1.6 Hz, 1H), 6.71 (d, J=8.0 Hz, 1H), 4.96 (s, 2H), 3.92 (d, J=6 Hz, 2H), 3.87-3.82 (m, 2H), 3.24 (s, 2H), 3.06-2.99 (m, 2H), 2.93 (s, 2H), 2.85 (s, 2H), 2.67-2.61 (m, 1H), 2.00 (s, 3H), 1.94-1.89 (m, 2H), 1.75 (s, 2H), 1.67 (s, 2H), 1.64-1.55 (m, 2H), 1.06-0.99 (m, 9H)

### Step F. Procedure for Preparation of tert-butyl 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-methylphenyl)picolinate

A mixture of 2-[2-[[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]methyl]-7-azaspiro[3.5]nonan-7-yl] acetic acid (101 mg, 128.18 μmol, 1 equiv.), 3-(6-amino-1H-indazol-3-yl)piperidine-2,6-dione (39.73 mg, 153.81 μmol, 1.2 equiv.), and DIEA (49.70 mg, 384.54 μmol, 66.98 μL, 3 equiv.) in DMF (1 mL) was degassed and purged with N₂ three times. The mixture was stirred at 25° C. for 5 minutes. After 5 minutes, HATU (73.11 mg, 192.27 μmol, 1.5 equiv.) was added, and the mixture was stirred at 25° C. for 2 hours under N₂ atmosphere. To the mixture was added water (2 mL) dropwise, and a pink solid was precipitated and collected by filtration. The pink solid was dissolved in DCM (10 mL) and concentrated under reduced pressure to give tert-butyl 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-methylphenyl)picolinate (116 mg, crude).

MS (ESI) m/z: 1028.6 [M+H]⁺

¹HNMR (400 MHz, DMSO) δ=12.90-12.79 (m, 1H), 10.89 (s, 1H), 10.53-10.18 (m, 1H), 8.02 (d, J=4.0 Hz, 1H), 8.01 (s, 1H), 7.78 (d, J=8.0 Hz, 1H), 7.67 (d, J=8.4 Hz, 1H), 7.60 (d, J=7.6 Hz, 1H), 7.47 (d, J=8.4 Hz, 1H), 7.44-7.43 (m, 1H), 7.40 (d, J=7.6 Hz, 1H), 7.36 (d, J=5.6 Hz, 1H), 7.33 (d, J=7.6 Hz, 1H), 7.17 (d, J=8.8 Hz, 1H), 6.92 (d, J=9.2 Hz, 1H), 6.86 (d, J=8.4 Hz, 1H), 6.82 (s, 1H), 6.73 (d, J=8.4 Hz, 1H), 4.97 (s, 2H), 4.33 (dd, J=9.6, 5.2 Hz, 1H), 3.87-3.84 (m, 2H), 3.05-3.02 (m, 2H), 2.66 (dd, J=9.6, 4.8 Hz, 2H), 2.39-2.33 (m, 1H), 2.25-2.11 (m, 2H), 2.01 (s, 3H), 1.94 (d, J=10.4 Hz, 2H), 1.89 (s, 2H), 1.81 (s, 2H), 1.73 (s, 2H), 1.68 (s, 2H), 1.66-1.59 (m, 2H), 1.23 (s, 2H), 1.20-1.14 (m, 2H), 1.04 (s, 9H)

### Step G. Procedure for Preparation of 6-(8-(benzo[d]thiazol-2-ylcarbamoyl)-3, 4-dihydroisoquinolin-2(1H)-yl)-3-(4-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(4-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-methylphenyl)picolinate (116 mg, 112.82 μmol, 1 equiv.) in DCM (1 mL) and TFA (1 mL) was degassed and purged with N₂ three times, and then the mixture was stirred at 25° C. for 16 hours under N₂ atmosphere. The reaction mixture was filtered and concentrated under reduced pressure to give a residue. The residue was purified by prep-HPLC to give 6-(8-(benzo[d]thiazol-2-ylcarbamoyl)-3, 4-dihydroisoquinolin-2(1H)-yl)-3-(4-((7-(2-((3-(2,6-dioxopiperidin-3-yl)-1-methyl-1H-indazol-6-yl)amino)-2-oxoethyl)-7-azaspiro[3.5]nonan-2-yl)methoxy)-2-methylphenyl)picolinic acid (31.3 mg, 32.2 μmol, 28.5% yield) as a yellow solid.

MS (ESI) m/z: 487.1 [M12+H]⁺

¹HNMR (400 MHz, DMSO) δ=10.87 (s, 1H), 9.83 (s, 1H), 8.20-8.14 (m, 1H), 8.024 (d, J=8.8 Hz, 1H), 7.78 (d, J=8 Hz, 1H), 7.63 (d, J=4.4 Hz, 1H), 7.61 (d, J=2.8 Hz, 1H), 7.47 (d, J=7.6 Hz, 1H), 7.45 (s, 1H), 7.43 (s, 1H), 7.39 (d, J=6.8 Hz, 1H), 7.36 (s, 1H), 7.33 (d, J=6.8 Hz, 1H), 7.23 (d, J=10 Hz, 1H), 6.93 (d, J=4.0 Hz, 1H), 6.91 (d, J=3.6 Hz, 1H), 6.78 (s, 1H), 6.69 (d, J=8.8 Hz, 1H), 4.96 (s, 2H), 4.32 (dd, J=9.6, 4.8 Hz, 1H), 3.95-3.87 (m, 8H), 3.01 (t, J=9.6 Hz, 2H), 2.68-2.64 (m, 2H), 2.64-2.60 (m, 2H), 2.43-2.37 (m, 2H), 2.33 (s, 2H), 2.24-2.11 (m, 2H), 2.02 (s, 3H), 1.92 (t, J=9.6 Hz, 2H), 1.66-1.72 (m, 2H), 1.63-1.57 (m, 4H)

### Example 202. Preparation of Compound 277

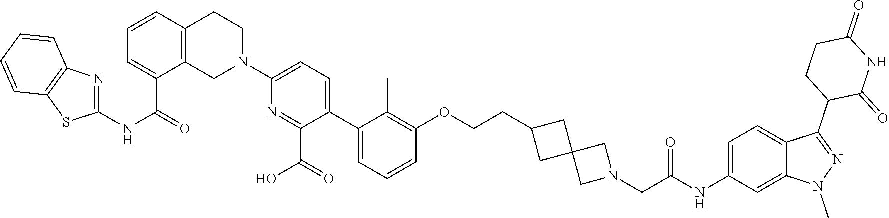 [SMILES: Cc1c(OCCC2CC3(C2)CN(CC(=O)Nc2ccc4c(C5CCC(=O)NC5=O)nn(C)c4c2)C3)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(2-(2-(2-((3-(2,6-dioxopiperidin-3-yl)-1-methyl-1H-indazol-6-yl)amino)-2-oxoethyl)-2-azaspiro[3.3]heptan-6-yl)ethoxy)-2-methylphenyl)picolinic acid

### Step A. Procedure for Preparation of tert-butyl 6-(2-methoxy-2-oxoethyl)-2-azaspiro[3.3]heptane-2-carboxylate

To a mixture of 2-(2-(tert-butoxycarbonyl)-2-azaspiro[3.3]heptan-6-yl)acetic acid (500 mg, 1.96 mmol, 1 equiv.) and Cs₂CO₃ (765.71 mg, 2.35 mmol, 1.2 equiv.) in DMF (1 mL) was added Mel (333.57 mg, 2.35 mmol, 146.30 uL, 1.2 equiv.). The mixture was stirred at 25° C. for 2 hours, diluted with water (10 mL), and extracted with EtOAc (5 mL×3). The combined organic layers were washed with brine (5 mL×2), dried over Na₂SO₄, filtered, and concentrated under reduced pressure to give a residue. The residue was purified by flash silica gel chromatography (eluent of 0˜36% ethyl acetate/petroleum ether) to give tert-butyl 6-(2-methoxy-2-oxoethyl)-2-azaspiro[3.3]heptane-2-carboxylate (400 mg, 1.4 mmol, 75.8% yield) as a white solid.

MS (ESI) m/z: 214.2 [M+H]⁺.

### Step B. Procedure for Preparation of tert-butyl 6-(2-hydroxyethyl)-2-azaspiro[3.3]heptane-2-carboxylate

A mixture of tert-butyl 6-(2-methoxy-2-oxoethyl)-2-azaspiro[3.3]heptane-2-carboxylate (400 mg, 1.49 mmol, 1 equiv.), LiAlH₄ (67.63 mg, 1.78 mmol, 1.2 equiv.) in THE (1 mL) was degassed and purged with N₂ three times, and then the mixture was stirred at 0° C. for 1 hour under N₂ atmosphere. The reaction mixture was quenched by the addition of H₂O (0.135 mL), 15% NaOH (0.405 mL), and H₂O 0.135 mL). The resulting mixture was then diluted with water (3 mL) and extracted with ethyl acetate (2 mL×3). The combined organic layers were dried over Na₂SO₄, filtered, and concentrated under reduced pressure to give a residue. The residue was purified by flash silica gel chromatography (eluent of 0˜8% DCM/MeOH) to give tert-butyl 6-(2-hydroxyethyl)-2-azaspiro[3.3]heptane-2-carboxylate (350 mg, 1.4 mmol, 97.6% yield) as a white solid.

MS (ESI) m/z: 186.3 [M+H]⁺.

### Step C. Procedure for Preparation of tert-butyl 6-(2-(3-bromo-2-methylphenoxy)ethyl)-2-azaspiro[3.3]heptane-2-carboxylate

To a solution of tert-butyl 6-(2-hydroxyethyl)-2-azaspiro[3.3]heptane-2-carboxylate (350 mg, 1.45 mmol, 1 equiv.) and 3-bromo-2-methyl-phenol (325.51 mg, 1.74 mmol, 1.2 equiv.) in toluene (15 mL) was added 2-(tributyl-X⁵-phosphanylidene)acetonitrile (700.08 mg, 2.90 mmol, 2 equiv.). The mixture was stirred at 120° C. for 2 hours. The reaction mixture was filtered and concentrated under reduced pressure to give a residue. The residue was purified by flash silica gel chromatography (eluent of 0˜18% ethyl acetate/petroleum ether) to give tert-butyl 6-(2-(3-bromo-2-methylphenoxy)ethyl)-2-azaspiro[3.3]heptane-2-carboxylate (300 mg, 731.0 μmol, 50.4% yield) as a white solid.

MS (ESI) m/z: 354.9 [M+H]⁺.

### Step D. Procedure for Preparation of 6-(2-(3-bromo-2-methylphenoxy)ethyl)-2-azaspiro[3.3]heptane

A solution of tert-butyl 6-(2-(3-bromo-2-methylphenoxy)ethyl)-2-azaspiro[3.3]heptane-2-carboxylate (200 mg, 487.40 μmol, 1 equiv.) in TFA (1 mL) and DCM (3 mL) was stirred at 25° C. for 1 hour. The reaction mixture was filtered and concentrated under reduced pressure to give a residue. The residue was purified by flash silica gel chromatography (eluent of 0˜68% ethyl acetate/petroleum ether) to give 6-(2-(3-bromo-2-methylphenoxy)ethyl)-2-azaspiro[3.3]heptane (250 mg, crude) as a white oil.

MS (ESI) m/z: 310.0 [M+H]⁺.

### Step E. Procedure for Preparation of ethyl 2-(6-(2-(3-bromo-2-methylphenoxy)ethyl)-2-azaspiro[3.3]heptan-2-yl)acetate

A mixture of 6-(2-(3-bromo-2-methylphenoxy)ethyl)-2-azaspiro[3.3]heptane (250 mg, 805.86 μmol, 1 equiv.) and ethyl 2-oxoacetate (98.72 mg, 967.03 μmol, 1.2 equiv.) in DCM (4 mL) was degassed and purged with N₂ three times, and then the mixture was stirred at 25° C. for 8 hours under N₂ atmosphere. NaBH(OAc)₃ (170.79 mg, 805.86 μmol, 1 equiv.) was added to the mixture, which was stirred at 25° C. for 1 hour. The reaction mixture was filtered, and the filtrate was concentrated under reduced pressure to give a residue. The residue was purified by flash silica gel chromatography (eluent of 0˜67% ethyl acetate/petroleum ether) to give ethyl 2-(6-(2-(3-bromo-2-methylphenoxy)ethyl)-2-azaspiro[3.3]heptan-2-yl)acetate (250 mg, 630.8 μmol, 78.2% yield) as a white oil.

MS (ESI) m/z: 396.2 [M+H]⁺.

### Step F. Procedure for Preparation of tert-butyl 6-(8-(benzo[d]thiazol-2-ylcarbamoyl)-3,4-dihydroisoquinolin-2(1H)-yl)-3-(3-(2-(2-(2-ethoxy-2-oxoethyl)-2-azaspiro[3.3]heptan-6-yl)ethoxy)-2-methylphenyl)picolinate

A mixture of ethyl 2-(6-(2-(3-bromo-2-methylphenoxy)ethyl)-2-azaspiro[3.3]heptan-2-yl)acetate (210 mg, 529.88 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (421.95 mg, 688.84 μmol, 1.3 equiv.), [2-(2-aminophenyl)phenyl]palladium(1⁺); bis(1-adamantyl)-butyl-phosphane; methanesulfonate (38.59 mg, 52.99 μmol, 0.1 equiv.), and K₂CO₃ (1.5 M, 529.88 μL, 1.5 equiv.) in dioxane (3 mL) was degassed and purged with N₂ three times. The mixture was stirred at 100° C. for 1 hour under N₂ atmosphere under microwave. The combined organic layers were filtered and concentrated under reduced pressure to give a residue. The residue was purified by flash silica gel chromatography (eluent of 0˜65% ethyl acetate/petroleum ether) to give tert-butyl 6-(8-(benzo[d]thiazol-2-ylcarbamoyl)-3,4-dihydroisoquinolin-2(1H)-yl)-3-(3-(2-(2-(2-ethoxy-2-oxoethyl)-2-azaspiro[3.3]heptan-6-yl)ethoxy)-2-methylphenyl)picolinate (200 mg, 249.3 μmol, 47.0% yield) as a yellow oil.

MS (ESI) m/z: 802.3 [M+H]⁺.

### Step G. Procedure for Preparation of 2-(6-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethyl)-2-azaspiro[3.3]heptan-2-yl)acetic acid

A mixture of tert-butyl 6-(8-(benzo[d]thiazol-2-ylcarbamoyl)-3,4-dihydroisoquinolin-2(1H)-yl)-3-(3-(2-(2-(2-ethoxy-2-oxoethyl)-2-azaspiro[3.3]heptan-6-yl)ethoxy)-2-methylphenyl)picolinate (200 mg, 249.38 μmol, 1 equiv.), and LiOH·H₂O (52.32 mg, 1.25 mmol, 5 equiv.) in THF (2 mL) and H₂O (1 mL) was stirred at 25° C. for 12 hours. The reaction mixture was diluted with H₂O (5 mL) and concentrated as a turbid liquid. Then the pH of the turbid liquid was adjusted to 3 with 1 N HCl (5 mL), and the residue was extracted with DCM/MEOH (20:1), The combined organic layers were filtered and concentrated. The residue was used in the next step without further purification. The compound 2-(6-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethyl)-2-azaspiro[3.3]heptan-2-yl)acetic acid (196 mg, crude) was obtained as a yellow solid.

MS (ESI) m/z: 774.3 [M+H]⁺.

### Step H. Procedure for Preparation of tert-butyl 6-(8-(benzo[d]thiazol-2-ylcarbamoyl)-3,4-dihydroisoquinolin-2(1H)-yl)-3-(3-(2-(2-(2-((3-(2,6-dioxopiperidin-3-yl)-1-methyl-1H-indazol-6-yl)amino)-2-oxoethyl)-2-azaspiro[3.3]heptan-6-yl)ethoxy)-2-methylphenyl)picolinate

A mixture of 2-(6-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)ethyl)-2-azaspiro[3.3]heptan-2-yl)acetic acid (176 mg, 227.41 μmol, 1 equiv.), 3-(6-amino-1-methyl-indazol-3-yl)piperidine-2,6-dione (70.48 mg, 272.89 μmol, 1.2 equiv.), and EDCI (65.39 mg, 341.11 μmol, 1.5 equiv.) in pyridine (1 mL) was stirred at 25° C. for 2 hours. The reaction mixture was diluted with H₂O (10 mL×3) and extracted with ethyl acetate (8 mL×3). The combined organic layers were filtered and concentrated under reduced pressure to give a residue. The residue was used in the next step without further purification. The compound tert-butyl 6-(8-(benzo[d]thiazol-2-ylcarbamoyl)-3,4-dihydroisoquinolin-2(1H)-yl)-3-(3-(2-(2-(2-((3-(2,6-dioxopiperidin-3-yl)-1-methyl-1H-indazol-6-yl)amino)-2-oxoethyl)-2-azaspiro[3.3]heptan-6-yl)ethoxy)-2-methylphenyl)picolinate (150 mg, 147.9 μmol, 65.0% yield) was obtained as a yellow oil.

MS (ESI) m/z: 1014.3 [M+H]⁺.

### Step I. Procedure for preparation of 6-(8-(benzo[d]thiazol-2-ylcarbamoyl)-3,4-dihydroisoquinolin-2(1H)-yl)-3-(3-(2-(2-(2-((3-(2,6-dioxopiperidin-3-yl)-1-methyl-1H-indazol-6-yl)amino)-2-oxoethyl)-2-azaspiro[3.3]heptan-6-yl)ethoxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(3-(2-(2-(2-((3-(2,6-dioxopiperidin-3-yl)-1-methyl-1H-indazol-6-yl)amino)-2-oxoethyl)-2-azaspiro[3.3]heptan-6-yl)ethoxy)-2-methylphenyl)picolinate (150 mg, 147.90 μmol, 1 equiv.) in TFA (1 mL) and DCM (1 mL) was stirred at 25° C. for 16 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(2-(2-(2-((3-(2,6-dioxopiperidin-3-yl)-1-methyl-1H-indazol-6-yl)amino)-2-oxoethyl)-2-azaspiro[3.3]heptan-6-yl)ethoxy)-2-methylphenyl)picolinic acid (11.8 mg, 12.3 μmol, 8.3% yield, 99.6% purity) as a yellow solid.

MS (ESI) m/z: 958.3 [M+H]⁺.

¹H NMR (400 MHz, DMSO-d₆) δ=10.87 (s, 1H), 9.84 (s, 1H), 8.04-7.99 (m, 2H), 7.80-7.75 (m, 1H), 7.64-7.59 (m, 2H), 7.47-7.32 (m, 5H), 7.19-7.15 (m, 1H), 7.10-7.04 (m, 1H), 6.92-6.82 (m, 2H), 6.69-6.59 (m, 1H), 5.04-4.93 (m, 2H), 4.31 (dd, J=4.8, 9.6 Hz, 1H), 3.92-3.89 (m, 6H), 3.23-3.21 (m, 4H), 3.04-3.00 (m, 2H), 2.69-2.62 (m, 4H), 2.34-2.27 (m, 4H), 2.23-2.10 (m, 2H), 1.89 (s, 3H), 1.85-1.78 (m, 4H)

### Example 203. Preparation of Compound 286

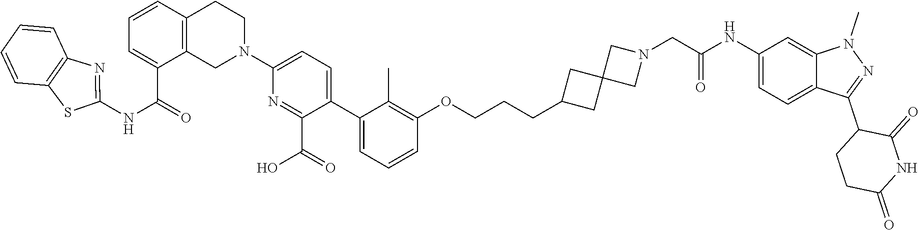 [SMILES: Cc1c(OCCCC2CC3(C2)CN(CC(=O)Nc2ccc4c(C5CCC(=O)NC5=O)nn(C)c4c2)C3)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[2-[2-[[3-(2,6-dioxo-3-piperidyl)-1-methyl-indazol-6-yl]amino]-2-oxo-ethyl]-2-azaspiro[3.3]heptan-6-yl]propoxy]-2-methyl-phenyl]pyridine-2-carboxylic acid

### Step A. Procedure for Preparation of tert-butyl 6-[(E)-3-ethoxy-3-oxo-prop-1-enyl]-2-azaspiro[3.3]heptane-2-carboxylate

A mixture of tert-butyl 6-formyl-2-azaspiro[3.3]heptane-2-carboxylate (300 mg, 1.33 mmol, 1 equiv.) in THE (1 mL) was added LiHMDS (1 M, 1.60 mL, 1.2 equiv.) at −78° C. for 0.5 hour under N₂ atmosphere, and then ethyl 2-diethoxyphosphorylacetate (358.25 mg, 1.60 mmol, 317.04 μL, 1.2 equiv.) in THE (0.5 mL) was added dropwise at −78° C. The resulting mixture was stirred at 20° C. for 1.5 hours under N₂ atmosphere. The reaction mixture was quenched by addition of saturated NH₄Cl (5 mL) at 0° C., diluted with EtOAc (1 mL), and extracted with (1 mL×3). The combined organic layers were washed with H₂O (3 mL), dried over Na₂SO₄, filtered, and concentrated under reduced pressure to give a residue. The residue was purified by flash silica gel chromatography (eluent of 0˜20% ethyl acetate/petroleum ether) to give tert-butyl 6-[(E)-3-ethoxy-3-oxo-prop-1-enyl]-2-azaspiro[3.3]heptane-2-carboxylate (230 mg, 778.6 μmol, 58.4% yield) as a colorless oil.

MS (ESI) m/z: 240.1 [M−56+H]⁺.

### Step B. Procedure for Preparation of tert-butyl 6-(3-ethoxy-3-oxo-propyl)-2-azaspiro[3.3]heptane-2-carboxylate

A mixture of tert-butyl 6-[(E)-3-ethoxy-3-oxo-prop-1-enyl]-2-azaspiro[3.3]heptane-2-carboxylate (230 mg, 778.68 μmol, 1 equiv.), Pd/C (50 mg, 80% purity), in MeOH (2 mL) was stirred at 25° C. for 2 hours under H₂ atmosphere. The reaction mixture was filtered and concentrated under reduced pressure to give tert-butyl 6-(3-ethoxy-3-oxo-propyl)-2-azaspiro[3.3]heptane-2-carboxylate (300 mg, crude) as a colorless oil.

¹H NMR (400 MHz, DMSO-d₆) δ=4.03 (q, J=7.2 Hz, 2H), 3.85-3.66 (m, 4H), 2.22-2.14 (m, 4H), 2.03 (d, J=7.6, 15.4 Hz, 1H), 1.76-1.69 (m, 2H), 1.60-1.52 (m, 2H), 1.35 (s, 9H), 1.17 (t, J=7.2 Hz, 3H).

### Step C. Procedure for Preparation of tert-butyl 6-(3-hydroxypropyl)-2-azaspiro[3.3]heptane-2-carboxylate

To a mixture of tert-butyl 6-(3-ethoxy-3-oxo-propyl)-2-azaspiro[3.3]heptane-2-carboxylate (300 mg, 1.01 mmol, 1 equiv.) in THE (3 mL) was added LiAlH₄ (32.54 mg, 857.46 μmol, 0.85 equiv.). The reaction mixture was stirred at 0° C. for 2 hours and was quenched by addition Na₂SO₄·10H₂O (1 g) under 0° C. and N₂ atmosphere. The resulting mixture was filtered, and the filtrate was concentrated under reduced pressure to give a residue. The residue was then purified by flash silica gel chromatography (eluent of 0˜40% ethyl acetate/petroleum ether) to give tert-butyl 6-(3-hydroxypropyl)-2-azaspiro[3.3]heptane-2-carboxylate (210 mg, 822.3 μmol, 81.5% yield) as a colorless oil.

¹H NMR (400 MHz, DMSO-d₆) δ=4.31 (t, J=5.2 Hz, 1H), 3.85-3.68 (m, 4H), 3.37-3.32 (m, 2H), 2.24-2.16 (m, 2H), 2.07-1.98 (m, 1H), 1.74-1.65 (m, 2H), 1.35 (s, 9H), 1.33-1.28 (m, 4H)

### Step D. Procedure for Preparation of tert-butyl 6-[3-(3-bromo-2-methyl-phenoxy)propyl]-2-azaspiro[3.3]heptane-2-carboxylate

A mixture of tert-butyl 6-(3-hydroxypropyl)-2-azaspiro[3.3]heptane-2-carboxylate (210.00 mg, 822.3 μmol, 1.2 equiv.), 3-bromo-2-methyl-phenol (128.18 mg, 685.33 μmol, 1 equiv.), and 2-(tributyl-λ5-phosphanylidene)acetonitrile (248.11 mg, 1.03 mmol, 1.5 equiv.) in toluene (1 mL) was stirred at 120° C. for 16 hours. The reaction mixture was concentrated under reduced pressure to give a residue. The mixture was purified by flash silica gel chromatography (eluent of 0˜20% ethyl acetate/petroleum ether) to give tert-butyl 6-[3-(3-bromo-2-methyl-phenoxy)propyl]-2-azaspiro[3.3]heptane-2-carboxylate (290 mg, 683.3 μmol, 99.7% yield) as a colorless oil.

MS (ESI) m/z: 446.0[M+23]⁺.

### Step E. Procedure for Preparation of 6-[3-(3-bromo-2-methyl-phenoxy)propyl]-2-azaspiro[3.3]heptane

A mixture of tert-butyl 6-[3-(3-bromo-2-methyl-phenoxy)propyl]-2-azaspiro[3.3]heptane-2-carboxylate (290 mg, 683.36 μmol, 1 equiv.) in TFA (0.5 mL) and DCM (1.5 mL) was stirred at 25° C. for 2 hours. The reaction mixture was concentrated under reduced pressure to give 6-[3-(3-bromo-2-methyl-phenoxy)propyl]-2-azaspiro[3.3]heptane (200 mg, 616.8 μmol, 90.2% yield, crude) as a yellow oil.

MS (ESI) m/z: 325.0[M+H]⁺(⁸⁰Br).

### Step F. Procedure for Preparation of ethyl 2-[6-[3-(3-bromo-2-methyl-phenoxy)propyl]-2-azaspiro[3.3]heptan-2-yl]acetate

A mixture of 6-[3-(3-bromo-2-methyl-phenoxy)propyl]-2-azaspiro[3.3]heptane (180 mg, 555.12 μmol, 1 equiv.) and ethyl 2-oxoacetate (136.01 mg, 666.14 μmol, 50% purity, 1.2 equiv.) in DCM (2 mL) was stirred at 25° C. for 2 hours. To the mixture was added NaBH(OAc)₃ (352.96 mg, 1.67 mmol, 3 equiv.). The mixture was then stirred at 25° C. for 1 hour. The reaction mixture was concentrated under reduced pressure to give ethyl 2-[6-[3-(3-bromo-2-methyl-phenoxy)propyl]-2-azaspiro[3.3]heptan-2-yl]acetate (200 mg, crude) as a yellow oil.

MS (ESI) m/z: 411.0[M+H]⁺(⁸⁰Br)

### Step G. Procedure for Preparation of tert-butyl 6-[8-(1,3-benzothiazol-2-ylcarbamoyl)-3,4-dihydro-1H-isoquinolin-2-yl]-3-[3-[3-[2-(2-ethoxy-2-oxo-ethyl)-2-azaspiro[3.3]heptan-6-yl]propoxy]-2-methyl-phenyl]pyridine-2-carboxylate

Ethyl 2-[6-[3-(3-bromo-2-methyl-phenoxy)propyl]-2-azaspiro[3.3]heptan-2-yl]acetate (35 mg, 85.29 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (73.76 mg, 102.35 μmol, 1.2 equiv.), KF (14.87 mg, 255.88 μmol, 5.99 μL, 3 equiv.), and [2-(2-aminophenyl)phenyl]palladium(1+); bis(1-adamantyl)-butyl-phosphane; methanesulfonate (12.42 mg, 17.06 μmol, 0.2 equiv.) were taken up into a microwave tube in dioxane (0.3 mL) and H₂O (0.03 mL). The sealed tube was heated at 100° C. for 1 hour under microwave. The reaction mixture was concentrated under reduced pressure to give a residue. The residue was purified by prep-TLC (SiO2, petroleum ether:ethyl acetate=1:1). The compound tert-butyl 6-[8-(1,3-benzothiazol-2-ylcarbamoyl)-3,4-dihydro-1H-isoquinolin-2-yl]-3-[3-[3-[2-(2-ethoxy-2-oxo-ethyl)-2-azaspiro[3.3]heptan-6-yl]propoxy]-2-methyl-phenyl]pyridine-2-carboxylate (50 mg, 53.8 μmol, 63.1% yield, 87.9% purity) was obtained as a yellow oil.

MS (ESI) m/z: 816.3[M+H]⁺.

¹H NMR (400 MHz, CDCl₃-d) 6=7.88-7.82 (m, 1H), 7.57 (d, J=8.0 Hz, 1H), 7.47-7.40 (m, 1H), 7.38-7.33 (m, 3H), 7.08 (t, J=8.0 Hz, 1H), 6.91-6.88 (m, 1H), 6.78 (d, J=7.6 Hz, 1H), 6.69 (d, J=6.4 Hz, 1H), 5.30 (s, 1H), 4.18 (d, J=6.4 Hz, 1H), 4.12-4.07 (m, 2H), 3.94 (t, J=6.0 Hz, 2H), 3.10-3.00 (m, 2H), 2.39-2.25 (m, 2H), 2.21-2.11 (m, 1H), 2.08-2.00 (m, 1H), 1.98 (s, 3H), 1.84-1.74 (m, 2H), 1.73-1.67 (m, 3H), 1.29-1.24 (m, 6H), 1.14 (s, 9H), 0.93-0.79 (m, 2H), 0.07 (s, 6H)

### Step H. Procedure for Preparation of 2-[6-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-2-azaspiro[3.3]heptan-2-yl]acetic acid

To a solution of tert-butyl 6-[8-(1,3-benzothiazol-2-ylcarbamoyl)-3,4-dihydro-1H-isoquinolin-2-yl]-3-[3-[3-[2-(2-ethoxy-2-oxo-ethyl)-2-azaspiro[3.3]heptan-6-yl]propoxy]-2-methyl-phenyl]pyridine-2-carboxylate (50 mg, 61.27 μmol, 1 equiv.) in THF (1 mL) was added LiOH·H₂O (12.86 mg, 306.37 μmol, 5 equiv.) and H₂O (0.3 mL). The mixture was stirred at 25° C. for 16 hours. The reaction mixture was concentrated under reduced pressure to give a residue. The residue was treated with water (6 mL). The pH of the resulting mixture was adjusted to around 3 by progressively adding dilute HCl (1M). The mixture was filtered to give 2-[6-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-2-azaspiro[3.3]heptan-2-yl]acetic acid (25 mg, 30.6 μmol, 50.0% yield, 96.7% purity) as a yellow solid.

MS (ESI) m/z: 788.6 [M+H]⁺.

### Step I. Procedure for Preparation of tert-butyl 6-[8-(1,3-benzothiazol-2-ylcarbamoyl)-3,4-dihydro-1H-isoquinolin-2-yl]-3-[3-[3-[2-[2-[[3-(2,6-dioxo-3-piperidyl)-1-methyl-indazol-6-yl]amino]-2-oxo-ethyl]-2-azaspiro[3.3]heptan-6-yl]propoxy]-2-methyl-phenyl]pyridine-2-carboxylate

To a solution of 2-[6-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-2-azaspiro[3.3]heptan-2-yl]acetic acid (20 mg, 25.38 μmol, 1 equiv.) and 3-(6-amino-1-methyl-indazol-3-yl)piperidine-2,6-dione (7.87 mg, 30.46 μmol, 1.2 equiv.) in pyridine (0.3 mL) was added EDCI (5.84 mg, 30.46 μmol, 1.2 eq). The mixture was stirred at 25° C. for 2 hours. The reaction mixture was concentrated under reduced pressure to give a residue. The residue was diluted with water (10 mL) and filtered to give tert-butyl 6-[8-(1,3-benzothiazol-2-ylcarbamoyl)-3,4-dihydro-1H-isoquinolin-2-yl]-3-[3-[3-[2-[2-[[3-(2,6-dioxo-3-piperidyl)-1-methyl-indazol-6-yl]amino]-2-oxo-ethyl]-2-azaspiro[3.3]heptan-6-yl]propoxy]-2-methyl-phenyl]pyridine-2-carboxylate (20 mg, 17.0 μmol, 67.1% yield, 87.6% purity) as a white solid.

MS (ESI) m/z: 514.9[½M+H]⁺.

### Step J. Procedure for Preparation of 6-[8-(1,3-benzothiazol-2-ylcarbamoyl)-3,4-dihydro-1H-isoquinolin-2-yl]-3-[3-[3-[2-[2-[[3-(2,6-dioxo-3-piperidyl)-1-methyl-indazol-6-yl]amino]-2-oxo-ethyl]-2-azaspiro[3.3]heptan-6-yl]propoxy]-2-methyl-phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[3-[2-[2-[[3-(2,6-dioxo-3-piperidyl)-1-methyl-indazol-6-yl]amino]-2-oxo-ethyl]-2-azaspiro[3.3]heptan-6-yl]propoxy]-2-methyl-phenyl]pyridine-2-carboxylate (20 mg, 19.45 μmol, 1 equiv.) in TFA (0.2 mL) and DCM (1 mL) was stirred at 25° C. for 16 hours. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[3-[2-[2-[[3-(2,6-dioxo-3-piperidyl)-1-methyl-indazol-6-yl]amino]-2-oxo-ethyl]-2-azaspiro[3.3]heptan-6-yl]propoxy]-2-methyl-phenyl]pyridine-2-carboxylic acid (7.9 mg, 7.7 μmol, 39.5% yield, 94.4% purity) as a yellow solid.

MS (ESI) m/z: 486.8 [M/2+H]⁺.

¹H NMR (400 MHz, DMSO-d₆) δ=10.95-10.80 (m, 1H), 9.89-9.77 (m, 1H), 8.30 (s, 1H), 8.05-7.95 (m, 2H), 7.76 (d, J=8.0 Hz, 1H), 7.66-7.55 (m, 2H), 7.48-7.38 (m, 2H), 7.38-7.29 (m, 3H), 7.17 (dd, J=1.2, 8.8 Hz, 1H), 7.08-7.00 (m, 1H), 6.82 (dd, J=4.0, 8.4 Hz, 2H), 6.67 (d, J=7.6 Hz, 1H), 4.96 (s, 2H), 4.31 (dd, J=5.2, 9.6 Hz, 1H), 3.90 (s, 3H), 3.34 (s, 2H), 3.22 (d, J=10.4 Hz, 4H), 3.00 (t, J=5.2 Hz, 3H), 2.70-2.55 (m, 3H), 2.38-2.27 (m, 2H), 2.26-2.13 (m, 4H), 2.13-2.07 (m, 1H), 1.91 (s, 3H), 1.75-1.68 (m, 2H), 1.67-1.60 (m, 2H), 1.54-1.46 (m, 2H)

### Example 204. Preparation of Compound 268a

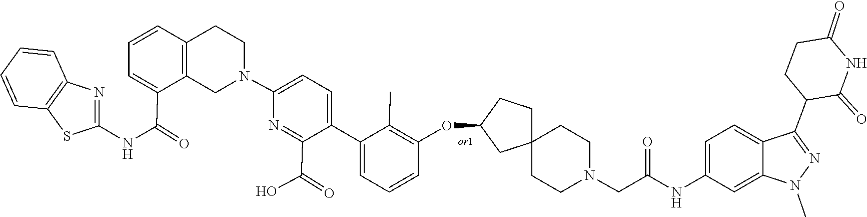 [SMILES: Cc1c(O[C@H]2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[[(3S)-8-[2-[[3-(2,6-dioxo-3-piperidyl)-1-methyl-indazol-6-yl]amino]-2-oxo-ethyl]-8-azaspiro[4.5]decan-3-yl]oxy]-2-methyl-phenyl]pyridine-2-carboxylic acid

### [rel-6-(8-(benzo[d]thiazol-2-ylcarbamoyl)-3,4-dihydroisoquinolin-2(1H)-yl)-3-(3-(((2S)-8-(2-((3-(2,6-dioxopiperidin-3-yl)-1-methyl-1H-indazol-6-yl)amino)-2-oxoethyl)-8-azaspiro[4.5]decan-2-yl)oxy)-2-methylphenyl)picolinic acid]

### Step A. Procedure for Preparation of tert-butyl 3-hydroxy-8-azaspiro[4.5]decane-8-carboxylate

To a solution of tert-butyl 3-oxo-8-azaspiro[4.5]decane-8-carboxylate (1 g, 3.95 mmol, 1 equiv.) in EtOH (10 mL) was added NaBH₄ (179.20 mg, 4.74 mmol, 1.2 equiv.) at 0° C. The mixture was stirred at 20° C. for 2 hours. The reaction mixture was quenched by addition saturated NH₄Cl (5 mL) at 0° C., and then diluted with ethyl acetate (1 mL) and extracted with 3 mL (1 mL×3). The combined organic layers were washed with H₂O (3 mL), dried over sodium sulfate, filtered, and concentrated under reduced pressure to give a residue. The residue was used in the next step without further purification. The compound tert-butyl 3-hydroxy-8-azaspiro[4.5]decane-8-carboxylate (1 g, crude) was obtained as a white oil.

### Step B. Procedure for Preparation of tert-butyl 3-(3-bromo-2-methyl-phenoxy)-8-azaspiro[4.5]decane-8-carboxylate

A mixture of 3-bromo-2-methyl-phenol (610.38 mg, 3.26 mmol, 1 equiv.), tert-butyl 3-hydroxy-8-azaspiro[4.5]decane-8-carboxylate (1 g, 3.92 mmol, 1.2 equiv.), and 2-(tributyl-λ⁵-phosphanylidene)acetonitrile (1.18 g, 4.89 mmol, 1.5 equiv.) in toluene (10 mL) was stirred at 120° C. for 16 hours. The reaction mixture was concentrated under reduced pressure to give a residue. The residue was purified by flash silica gel chromatography (eluent of 0˜10% ethyl acetate/petroleum ether) to give tert-butyl 3-(3-bromo-2-methyl-phenoxy)-8-azaspiro[4.5]decane-8-carboxylate (600 mg, 1.3 mmol, 39.9% yield, 92% purity) as a yellow oil.

MS (ESI) m/z: 367.9[M−56+H]⁺.

¹H NMR (400 MHz, DMSO-d₆) δ=7.15-7.11 (m, 1H), 7.10-7.05 (m, 1H), 6.93 (d, J=7.6 Hz, 1H), 4.88-4.84 (m, 1H), 3.30-3.24 (m, 4H), 2.20 (s, 3H), 2.16-2.02 (m, 2H), 1.94-1.88 (m, 1H), 1.80-1.73 (m, 1H), 1.69-1.63 (m, 2H), 1.58-1.49 (m, 2H), 1.48-1.45 (m, 2H), 1.38 (s, 9H)

### Step C. Procedure for Preparation of tert-butyl (S)-2-(3-bromo-2-methylphenoxy)-8-azaspiro[4.5]decane-8-carboxylate

The tert-butyl 3-(3-bromo-2-methyl-phenoxy)-8-azaspiro[4.5]decane-8-carboxylate residue was purified by prep-HPLC to give tert-butyl (R)-2-(3-bromo-2-methylphenoxy)-8-azaspiro[4.5]decane-8-carboxylate (200 mg, 471.29 μmol, 33.33% yield) and tert-butyl (S)-2-(3-bromo-2-methylphenoxy)-8-azaspiro[4.5]decane-8-carboxylate (220 mg, 518.4 μmol, 36.7% yield) as a colorless oil.

¹H NMR (400 MHz, DMSO-d₆) δ=7.14-7.11 (m, 1H), 7.10-7.05 (m, 1H), 6.93 (d, J=8.0 Hz, 1H), 4.88-4.84 (m, 1H), 3.31-3.23 (m, 4H), 2.20 (s, 3H), 2.12-2.01 (m, 1H), 1.93-1.88 (m, 1H), 1.81-1.74 (m, 1H), 1.69-1.63 (m, 2H), 1.58-1.44 (m, 4H), 1.38 (s, 10H)

¹H NMR (400 MHz, DMSO-d₆) δ=7.14-7.11 (m, 1H), 7.10-7.05 (m, 1H), 6.93 (d, J=8.0 Hz, 1H), 4.88-4.84 (m, 1H), 3.30-3.24 (m, 4H), 2.20 (s, 3H), 2.10-2.04 (m, 1H), 1.93-1.88 (m, 1H), 1.82-1.73 (m, 1H), 1.69-1.63 (m, 2H), 1.58-1.46 (m, 4H), 1.38 (s, 10H)

### Step D. Procedure for Preparation of (S)-2-(3-bromo-2-methylphenoxy)-8-azaspiro[4.5]decane

A solution of tert-butyl (S)-2-(3-bromo-2-methylphenoxy)-8-azaspiro[4.5]decane-8-carboxylate (200 mg, 471.29 μmol, 1 equiv.) in HCl/dioxane (2 mL) was stirred at 25° C. for 1 hour. The reaction mixture was concentrated under reduced pressure to give a residue. The residue was used in the next step without further purification. The compound (S)-2-(3-bromo-2-methylphenoxy)-8-azaspiro[4.5]decane (150 mg, crude) was obtained as a colorless oil.

MS (ESI) m/z: 324.1[M+H]⁺

### Step E. Procedure for Preparation of ethyl (S)-2-(2-(3-bromo-2-methylphenoxy)-8-azaspiro[4.5]decan-8-yl)acetate

A mixture of (S)-2-(3-bromo-2-methylphenoxy)-8-azaspiro[4.5]decane (140 mg, 431.76 μmol, 1 equiv.), ethyl 2-bromoacetate (72.10 mg, 431.76 μmol, 47.75 μL, 1 equiv.), K₂CO₃ (179.01 mg, 1.30 mmol, 3 equiv.), and KI (35.84 mg, 215.88 μmol, 0.5 equiv.) in CH₃CN (2 mL) was stirred at 60° C. for 2 hours. The reaction mixture was filtered and concentrated under reduced pressure to give a residue. The residue was purified by flash silica gel chromatography (eluent of 0˜40% ethyl acetate/petroleum ether) to give ethyl (S)-2-(2-(3-bromo-2-methylphenoxy)-8-azaspiro[4.5]decan-8-yl)acetate (110 mg, 268.0 μmol, 62.0% yield) as a yellow oil.

MS (ESI) m/z: 410.0[M+H]⁺¹H NMR (400 MHz, DMSO-d₆) δ=7.14-7.11 (m, 1H), 7.10-7.05 (m, 1H), 6.93 (d, J=8.0 Hz, 1H), 4.87-4.82 (m, 1H), 4.09-4.04 (m, 2H), 3.16 (s, 2H), 2.48-2.35 (m, 4H), 2.19 (s, 3H), 2.10-2.00 (m, 1H), 1.88-1.83 (m, 1H), 1.81-1.71 (m, 1H), 1.65-1.51 (m, 5H), 1.45-1.43 (m, 2H), 1.17 (t, J=7.2 Hz, 3H)

### Step F. Procedure for Preparation of tert-butyl (S)-6-(8-(benzo[d]thiazol-2-ylcarbamoyl)-3,4-dihydroisoquinolin-2(1H)-yl)-3-(3-((8-(2-ethoxy-2-oxoethyl)-8-azaspiro[4.5]decan-2-yl)oxy)-2-methylphenyl)picolinate

A mixture of ethyl (S)-2-(2-(3-bromo-2-methylphenoxy)-8-azaspiro[4.5]decan-8-yl)acetate (100 mg, 243.70 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (179.13 mg, 292.44 μmol, 1.2 equiv.), [2-(2-aminophenyl)phenyl]palladium(1+); bis(1-adamantyl)-butyl-phosphane; methanesulfonate (35.50 mg, 48.74 μmol, 0.2 equiv.), and K₂CO₃ (1.5 M, 487.40 uL, 3 equiv.) in dioxane (2 mL) was degassed and purged with N₂ three times, and then the mixture was stirred at 100° C. for 1 hour under N₂ atmosphere under microwave. The reaction mixture was filtered and concentrated under reduced pressure to give a residue. The mixture was purified by flash silica gel chromatography (eluent of 0˜52% ethyl acetate/petroleum ether) to give tert-butyl (S)-6-(8-(benzo[d]thiazol-2-ylcarbamoyl)-3,4-dihydroisoquinolin-2(1H)-yl)-3-(3-((8-(2-ethoxy-2-oxoethyl)-8-azaspiro[4.5]decan-2-yl)oxy)-2-methylphenyl)picolinate (152 mg, 186.2 μmol, 76.4% yield) as a yellow oil.

MS (ESI) m/z: 816.3 [M+H]⁺

### Step G. Procedure for Preparation of (S)-2-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)-8-azaspiro[4.5]decan-8-yl)acetic acid

A mixture of tert-butyl (S)-6-(8-(benzo[d]thiazol-2-ylcarbamoyl)-3,4-dihydroisoquinolin-2(1H)-yl)-3-(3-((8-(2-ethoxy-2-oxoethyl)-8-azaspiro[4.5]decan-2-yl)oxy)-2-methylphenyl)picolinate (142 mg, 174.02 μmol, 1 equiv.) and LiOH·H₂O (36.51 mg, 870.08 μmol, 5 equiv.) in THE (2 mL) and H₂O (1 mL) was stirred at 25° C. for 16 hours. The reaction mixture was diluted with H₂O (5 mL) and concentrated as a turbid liquid. Then the pH of the turbid liquid was adjusted to 3 with 1 N HCl (5 mL), and the residue was extracted with DCM/MeOH (20:1). The combined organic layers were filtered and concentrated. The residue was used in the next step without further purification. The compound (S)-2-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)-8-azaspiro[4.5]decan-8-yl)acetic acid (140 mg, crude) was obtained as a yellow solid.

MS (ESI) m/z: 788.3 [M+H]⁺

### Step H. Procedure for Preparation of tert-butyl 6-(8-(benzo[d]thiazol-2-ylcarbamoyl)-3,4-dihydroisoquinolin-2(1H)-yl)-3-(3-(((2S)-8-(2-((3-(2,6-dioxopiperidin-3-yl)-1-methyl-1H-indazol-6-yl)amino)-2-oxoethyl)-8-azaspiro[4.5]decan-2-yl)oxy)-2-methylphenyl)picolinate

A mixture of (S)-2-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)-8-azaspiro[4.5]decan-8-yl)acetic acid (100 mg, 126.91 μmol, 1 equiv.), 3-(6-amino-1-methyl-indazol-3-yl)piperidine-2,6-dione (39.33 mg, 152.29 μmol, 1.2 equiv.), and EDCI (36.49 mg, 190.36 μmol, 1.5 equiv.) in pyridine (1 mL) was stirred at 25° C. for 2 hours. The mixture was diluted with H₂O mL (10 mL×3) and extracted with ethyl acetate 24 mL (8 mL×3). The combined organic layers were filtered and concentrated under reduced pressure to give a residue. The residue was used in the next step without further purification. The compound tert-butyl 6-(8-(benzo[d]thiazol-2-ylcarbamoyl)-3,4-dihydroisoquinolin-2(1H)-yl)-3-(3-(((2S)-8-(2-((3-(2,6-dioxopiperidin-3-yl)-1-methyl-1H-indazol-6-yl)amino)-2-oxoethyl)-8-azaspiro[4.5]decan-2-yl)oxy)-2-methylphenyl)picolinate (100 mg, 97.2 μmol, 76.6% yield) was obtained as a yellow solid.

MS (ESI) m/z: 1028.4[M+H]⁺.

### Step I. Procedure for Preparation of 6-[8-(1,3-benzothiazol-2-ylcarbamoyl)-3,4-dihydro-1H-isoquinolin-2-yl]-3-[3-[[(3S)-8-[2-[[3-(2,6-dioxo-3-piperidyl)-1-methyl-indazol-6-yl]amino]-2-oxo-ethyl]-8-azaspiro[4.5]decan-3-yl]oxy]-2-methyl-phenyl]pyridine-2-carboxylic acid [rel-6-(8-(benzo[d]thiazol-2-ylcarbamoyl)-3,4-dihydroisoquinolin-2(1H)-yl)-3-(3-(((2S)-8-(2-((3-(2,6-dioxopiperidin-3-yl)-1-methyl-1H-indazol-6-yl)amino)-2-oxoethyl)-8-azaspiro[4.5]decan-2-yl)oxy)-2-methylphenyl)picolinic acid]

A solution of tert-butyl 6-(8-(benzo[d]thiazol-2-ylcarbamoyl)-3,4-dihydroisoquinolin-2(1H)-yl)-3-(3-(((2S)-8-(2-((3-(2,6-dioxopiperidin-3-yl)-1-methyl-1H-indazol-6-yl)amino)-2-oxoethyl)-8-azaspiro[4.5]decan-2-yl)oxy)-2-methylphenyl)picolinate (90 mg, 87.53 μmol, 1 equiv.) in TFA (0.8 mL) and DCM (0.8 mL) was stirred at 25° C. for 16 hours. The reaction mixture was concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[[(3S)-8-[2-[[3-(2,6-dioxo-3-piperidyl)-1-methyl-indazol-6-yl]amino]-2-oxo-ethyl]-8-azaspiro[4.5]decan-3-yl]oxy]-2-methyl-phenyl]pyridine-2-carboxylic acid [rel-6-(8-(benzo[d]thiazol-2-ylcarbamoyl)-3,4-dihydroisoquinolin-2(1H)-yl)-3-(3-(((2S)-8-(2-((3-(2,6-dioxopiperidin-3-yl)-1-methyl-1H-indazol-6-yl)amino)-2-oxoethyl)-8-azaspiro[4.5]decan-2-yl)oxy)-2-methylphenyl)picolinic acid](24.2 mg, 24.1 μmol, 27.5% yield, 96.0% purity) as a white solid.

MS (ESI) m/z: 972.3[M+H]⁺

¹H NMR (400 MHz, DMSO-d₆) δ=10.95-10.82 (m, 1H), 9.89-9.79 (m, 1H), 8.07-7.99 (m, 2H), 7.78 (d, J=8.0 Hz, 1H), 7.62 (d, J=8.8 Hz, 2H), 7.50-7.42 (m, 3H), 7.40-7.32 (m, 2H), 7.22 (d, J=8.8 Hz, 1H), 7.08 (t, J=8.0 Hz, 1H), 6.94 (d, J=8.4 Hz, 1H), 6.85 (d, J=8.4 Hz, 1H), 6.61 (d, J=7.6 Hz, 1H), 4.97 (s, 2H), 4.79-4.70 (m, 1H), 4.32 (dd, J=5.2, 9.6 Hz, 1H), 3.95-3.88 (m, 5H), 3.13 (s, 2H), 3.06-2.99 (m, 2H), 2.69-2.59 (m, 4H), 2.54 (s, 2H), 2.39-2.27 (m, 2H), 2.21-2.14 (m, 1H), 2.11-2.03 (m, 2H), 1.96-1.90 (m, 1H), 1.86 (s, 3H), 1.71-1.63 (m, 4H), 1.55 (d, J=5.2 Hz, 2H)

### Example 205. Preparation of Compound 268b

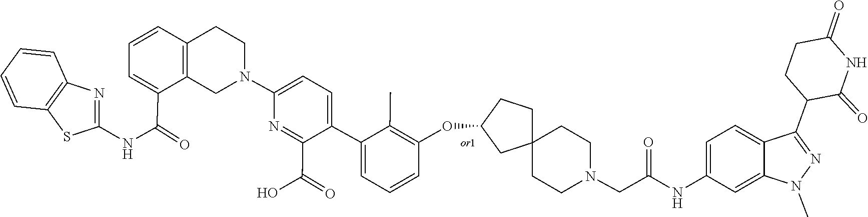 [SMILES: Cc1c(O[C@@H]2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O]

### 6-(8-(benzo[d]thiazol-2-yl-carbamoyl)-3,4dihydroisoquinolin-2(1H)-yl)-3-(3-(((2R)-8-(2-((3-(2,6-dioxopiperidin-3-yl)-1-methyl-1H-indazol-6-yl)amino)-2-oxoethyl)-8-azaspiro[4.5]decan-2-yl)oxy)-2-methylphenyl)picolinic acid

### [rel-6-(8-(benzo[d]thiazol-2-ylcarbamoyl)-3,4-dihydroisoquinolin-2(1H)-yl)-3-(3-(((2R)-8-(2-((3-(2,6-dioxopiperidin-3-yl)-1-methyl-1H-indazol-6-yl)amino)-2-oxoethyl)-8-azaspiro[4.5]decan-2-yl)oxy)-2-methylphenyl)picolinic acid]

### Step A. Procedure for Preparation of (3R)-3-(3-bromo-2-methyl-phenoxy)-8-azaspiro[4.5]decane

A mixture of tert-butyl (3R)-3-(3-bromo-2-methyl-phenoxy)-8-azaspiro[4.5]decane-8-carboxylate (200 mg, 471.29 μmol, 1 equiv.) in HCl/dioxane (2 mL) was stirred at 25° C. for 2 hours. The reaction mixture was concentrated under reduced pressure to give a residue. The residue was used in the next step without purification. The compound (3R)-3-(3-bromo-2-methyl-phenoxy)-8-azaspiro[4.5]decane (150 mg, crude) was obtained as a white solid.

MS (ESI) m/z: 325.9 [M+H]⁺

### Step B. Procedure for Preparation of ethyl (R)-2-(2-(3-bromo-2-methylphenoxy)-8-azaspiro[4.5]decan-8-yl)acetate

A solution of (3R)-3-(3-bromo-2-methyl-phenoxy)-8-azaspiro[4.5]decane (150 mg, 462.60 μmol, 1 equiv.), ethyl 2-bromoacetate (77.25 mg, 462.60 μmol, 51.16 μL, 1 equiv.), K₂CO₃ (191.80 mg, 1.39 mmol, 3 equiv.), and KI (38.40 mg, 231.30 μmol, 0.5 equiv.) in CH₃CN (2 mL) was stirred at 60° C. for 2 hours. The reaction mixture was filtered and concentrated under reduced pressure to give a residue. The residue was then purified by flash silica gel chromatography (eluent of 0˜40% ethyl acetate/petroleum ether) to give ethyl (R)-2-(2-(3-bromo-2-methylphenoxy)-8-azaspiro[4.5]decan-8-yl)acetate (150 mg, 365.5 μmol, 79.0% yield) as a yellow oil.

MS (ESI) m/z: 411.9 [M+H]⁺

¹H NMR (400 MHz, DMSO-d₆) δ=7.14-7.04 (m, 2H), 6.93 (d, J=8.0 Hz, 1H), 4.84 (dd, J=2.8, 6.0 Hz, 1H), 4.06 (d, J=7.2 Hz, 2H), 3.16 (s, 2H), 2.46-2.39 (m, 4H), 2.21-2.16 (m, 1H), 2.19 (s, 3H), 2.06-2.00 (m, 1H), 1.88-1.82 (m, 1H), 1.65-1.59 (m, 2H), 1.58-1.49 (m, 4H), 1.47-1.42 (m, 2H), 1.19-1.15 (m, 3H)

### Step C. Procedure for Preparation of tert-butyl 6-[8-(1,3-benzothiazol-2-ylcarbamoyl)-3,4-dihydro-1H-isoquinolin-2-yl]-3-[3-[[(3R)-8-(2-ethoxy-2-oxo-ethyl)-8-azaspiro[4.5]decan-3-yl]oxy]-2-methyl-phenyl]pyridine-2-carboxylate

A mixture of ethyl (R)-2-(2-(3-bromo-2-methylphenoxy)-8-azaspiro[4.5]decan-8-yl)acetate (150 mg, 365.55 μmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (268.70 mg, 438.66 μmol, 1.2 equiv.), K₂CO₃ (1.5 M, 731.09 μL, 3 equiv.), and [2-(2-aminophenyl)phenyl]palladium(1⁺); bis(1-adamantyl)-butyl-phosphane; methane sulfonate (53.24 mg, 73.11 μmol, 0.2 equiv.) in dioxane (1.5 mL) was degassed and purged with N₂ three times, and then the mixture was stirred at 100° C. for 1 hour under N₂ atmosphere under microwave. The reaction mixture was filtered and concentrated under reduced pressure to give a residue. The residue was purified by flash silica gel chromatography (eluent of 0˜52% ethyl acetate/petroleum ether) to give tert-butyl 6-[8-(1,3-benzothiazol-2-ylcarbamoyl)-3,4-dihydro-1H-isoquinolin-2-yl]-3-[3-[[(3R)-8-(2-ethoxy-2-oxo-ethyl)-8-azaspiro[4.5]decan-3-yl]oxy]-2-methyl-phenyl]pyridine-2-carboxylate (200 mg, 242.6 μmol, 66.3% yield, 99% purity) as a yellow oil.

MS (ESI) m/z: 816.7 [M+H]⁺.

### Step D. Procedure for Preparation of (R)-2-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)-8-azaspiro[4.5]decan-8-yl)acetic acid

A mixture of give tert-butyl 6-[8-(1,3-benzothiazol-2-ylcarbamoyl)-3,4-dihydro-1H-isoquinolin-2-yl]-3-[3-[[(3R)-8-(2-ethoxy-2-oxo-ethyl)-8-azaspiro[4.5]decan-3-yl]oxy]-2-methyl-phenyl]pyridine-2-carboxylate (140 mg, 171.57 μmol, 1 equiv.), LiOH·H₂O (36.00 mg, 857.83 μmol, 5 equiv.) in THE (2 mL) and H₂O (1 mL) was stirred at 25° C. for 16 hours. The reaction mixture was diluted with H₂O (5 mL) and concentrated as a turbid liquid. Then the pH of the turbid liquid was adjusted to 3 with 1 N HCl (5 mL), the residue was extracted with DCM/MeOH (20:1), the combined organic layers were filtered and concentrated. The residue was used in the next step without further purification. The compound (R)-2-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)-8-azaspiro[4.5]decan-8-yl)acetic acid (200 mg, crude) was obtained as a yellow solid.

MS (ESI) m/z: 788.4 [M+H]⁺.

### Step E. Procedure for Preparation of tert-butyl 6-(8-(benzo[d]thiazol-2-ylcarbamoyl)-3,4-dihydroisoquinolin-2(1 h)-yl)-3-(3-(((2r)-8-(2-((3-(2,6-dioxopiperidin-3-yl)-1-methyl-1 h-indazol-6-yl)amino)-2-oxoethyl)-8-azaspiro[4.5]decan-2-yl)oxy)-2-methylphenyl)picolinate

A mixture of (R)-2-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)-8-azaspiro[4.5]decan-8-yl)acetic acid (120 mg, 152.29 μmol, 1 equiv.), 3-(6-amino-1-methyl-1H-indazol-3-yl)piperidine-2,6-dione (47.20 mg, 182.75 μmol, 1.2 equiv.), and EDCI (43.79 mg, 228.44 μmol, 1.5 equiv.) in pyridine (1.2 mL) was stirred at 25° C. for 2 hours. The reaction mixture was filtered and concentrated under reduced pressure to give a residue. The residue was used in the next step without further purification. The compound tert-butyl 6-(8-(benzo[d]thiazol-2-ylcarbamoyl)-3,4-dihydroisoquinolin-2(1 h)-yl)-3-(3-(((2r)-8-(2-((3-(2,6-dioxopiperidin-3-yl)-1-methyl-1 h-indazol-6-yl)amino)-2-oxoethyl)-8-azaspiro[4.5]decan-2-yl)oxy)-2-methylphenyl)picolinate (100 mg, crude) was obtained as a yellow oil.

MS (ESI) m/z: 1028.4[M+H]⁺.

### Step F. Procedure for Preparation of 6-(8-(benzo[d]thiazol-2-yl-carbamoyl)-3,4dihydroisoquinolin-2(1H)-yl)-3-(3-(((2R)-8-(2-((3-(2,6-dioxopiperidin-3-yl)-1-methyl-11H-indazol-6-yl)amino)-2-oxoethyl)-8-azaspiro[4.5]decan-2-yl)oxy)-2-methylphenyl)picolinic acid [rel-6-(8-(benzo[d]thiazol-2-ylcarbamoyl)-3,4-dihydroisoquinolin-2(1H)-yl)-3-(3-(((2R)-8-(2-((3-(2,6-dioxopiperidin-3-yl)-1-methyl-1H-indazol-6-yl)amino)-2-oxoethyl)-8-azaspiro[4.5]decan-2-yl)oxy)-2-methylphenyl)picolinic acid]

A mixture of tert-butyl 6-(8-(benzo[d]thiazol-2-ylcarbamoyl)-3,4-dihydroisoquinolin-2(1 h)-yl)-3-(3-(((2r)-8-(2-((3-(2,6-dioxopiperidin-3-yl)-1-methyl-1 h-indazol-6-yl)amino)-2-oxoethyl)-8-azaspiro[4.5]decan-2-yl)oxy)-2-methylphenyl)picolinate (90 mg, 87.53 μmol, 1 equiv.) in DCM (0.8 mL) and TFA (0.8 mL) was stirred at 25° C. for 16 hours. The reaction mixture was filtered and concentrated under reduced pressure to give a residue. The residue was purified by prep-HPLC to give 6-(8-(benzo[d]thiazol-2-yl-carbamoyl)-3,4dihydroisoquinolin-2(1H)-yl)-3-(3-(((2R)-8-(2-((3-(2,6-dioxopiperidin-3-yl)-1-methyl-1H-indazol-6-yl)amino)-2-oxoethyl)-8-azaspiro[4.5]decan-2-yl)oxy)-2-methylphenyl)picolinic acid [rel-6-(8-(benzo[d]thiazol-2-ylcarbamoyl)-3,4-dihydroisoquinolin-2(1H)-yl)-3-(3-(((2R)-8-(2-((3-(2,6-dioxopiperidin-3-yl)-1-methyl-1H-indazol-6-yl)amino)-2-oxoethyl)-8-azaspiro[4.5]decan-2-yl)oxy)-2-methylphenyl)picolinic acid] as a white solid.

MS (ESI) m/z: 972.5[M+H]⁺.

¹H NMR (400 MHz, DMSO-d₆) δ=10.90-10.84 (m, 1H), 9.82 (s, 1H), 8.16 (s, 1H), 8.06-8.01 (m, 2H), 7.78 (d, J=8.4 Hz, 1H), 7.62 (d, J=8.8 Hz, 2H), 7.48-7.42 (m, 1H), 7.35 (td, J=8.0, 10.4 Hz, 2H), 7.24-7.20 (m, 1H), 7.11-7.05 (m, 1H), 6.94 (J=8.4 Hz, 1H), 6.87-6.83 (m, 1H), 6.64-6.60 (m, 1H), 5.01-4.95 (m, 2H), 4.86-4.80 (m, 1H), 4.32 (dd, J=5.2, 10.0 Hz, 1H), 3.92 (s, 5H), 3.29-3.23 (m, 4H), 3.13 (s, 2H), 3.05-3.00 (m, 2H), 2.68-2.63 (m, 2H), 2.40-2.28 (m, 2H), 2.21-2.14 (m, 1H), 2.12-2.02 (m, 2H), 1.86 (s, 3H), 1.75-1.63 (m, 4H), 1.65-1.47 (m, 4H)

### Example 206. Preparation of Compound 272b

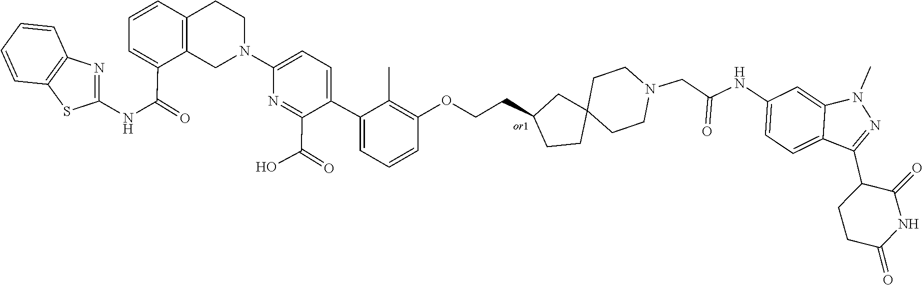 [SMILES: Cc1c(OCC[C@@H]2CCC3(CCN(CC(=O)Nc4ccc5c(C6CCC(=O)NC6=O)nn(C)c5c4)CC3)C2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydro-isoquinolin-2(1 h)-yl)-3-(3-(2-((2r)-8-(2-((3-(2,6-dioxopiperidin-3-yl)-1-methyl-1 h-indazol-6-yl)amino)-2-oxoethyl)-8-azaspiro[4.5]decan-2-yl)ethoxy)-2-methylphenyl)picolinic acid

### [rel-6-(8-(benzo[d]thiazol-2-ylcarbamoyl)-3,4-dihydroisoquinolin-2(1H)-yl)-3-(3-(2-((2R)-8-(2-((3-(2,6-dioxopiperidin-3-yl)-1-methyl-1H-indazol-6-yl)amino)-2-oxoethyl)-8-azaspiro[4.5]decan-2-yl)ethoxy)-2-methylphenyl)picolinic acid]

### Step A. Procedure for Preparation of tert-butyl (R)-6-(8-(benzo[d]thiazol-2-ylcarbamoyl)-3,4-dihydroisoquinolin-2(1H)-yl)-3-(3-(2-(8-(2-ethoxy-2-oxoethyl)-8-azaspiro[4.5]decan-2-yl)ethoxy)-2-methylphenyl) picolinate

A mixture of ethyl (R)-2-(2-(2-(3-bromo-2-methylphenoxy)ethyl)-8-azaspiro[4.5]decan-8-yl) acetate (97.81 mg, 159.67 μmol, 1.4 equiv.), ethyl 2-[(3R)-3-[2-(3-bromo-2-methylphenoxy)ethyl]-8-azaspiro[4.5]decan-8-yl]acetate (50 mg, 114.05 μmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (16.61 mg, 22.81 μmol, 0.2 equiv.), and K₂CO₃ (1.5 M, 114.05 μL, 1.5 equiv.) in dioxane (0.5 mL) was degassed and purged with N₂ three times, and then the mixture was stirred at 100° C. for 1 hour under microwave. The reaction mixture was filtered and concentrated under reduced pressure to give a residue. The residue was purified by flash silica gel chromatography (eluent of 0˜50% ethyl acetate/petroleum ether). The compound tert-butyl (R)-6-(8-(benzo[d]thiazol-2-ylcarbamoyl)-3,4-dihydroisoquinolin-2(1H)-yl)-3-(3-(2-(8-(2-ethoxy-2-oxoethyl)-8-azaspiro[4.5]decan-2-yl)ethoxy)-2-methylphenyl) picolinate (95 mg, 112.5 μmol, 98.6% yield) was obtained as a colorless oil.

MS (ESI) m/z: 844.4 [M+H]⁺

### Step B. Procedure for Preparation of (r)-2-(2-(2-(3-(6-(8-(benzo[d]thiazol-2-ylcarbamoyl)-3,4-dihydroisoquinolin-2(1 h)-yl)-2-(tert-butoxycarbonyl)pyridin-3-yl)-2-methylphenoxy)ethyl)-8-azaspiro[4.5]decan-8-yl)acetic acid

A mixture of tert-butyl (R)-6-(8-(benzo[d]thiazol-2-ylcarbamoyl)-3,4-dihydroisoquinolin-2(1H)-yl)-3-(3-(2-(8-(2-ethoxy-2-oxoethyl)-8-azaspiro[4.5]decan-2-yl)ethoxy)-2-methylphenyl)picolinate (95 mg, 112.55 μmol, 1 equiv.) and LiOH H₂O (14.17 mg, 337.65 μmol, 3 equiv.) in THF (3 mL) and H₂O (1 mL) was stirred at 25° C. for 10 hours. The mixture was concentrated and the pH was adjusted to 3, then triturated and filtered to obtain a residue. The residue was used in next step without further purification. The compound (r)-2-(2-(2-(3-(6-(8-(benzo[d]thiazol-2-ylcarbamoyl)-3,4-dihydroisoquinolin-2(1 h)-yl)-2-(tert-butoxycarbonyl)pyridin-3-yl)-2-methylphenoxy)ethyl)-8-azaspiro[4.5]decan-8-yl)acetic acid (80 mg, 98.0 μmol, 87.1% yield) was obtained as a white solid.

MS (ESI) m/z: 816.4 [M+H]⁺

### Step C. Procedure for Preparation of tert-butyl 6-(8-(benzo[d]thiazol-2-ylcarbamoyl)-3,4-dihydroisoquinolin-2(1H)-yl)-3-(3-(2-((2R)-8-(2-((3-(2,6-dioxopiperidin-3-yl)-1-methyl-11H-indazol-6-yl)amino)-2-oxoethyl)-8-azaspiro[4.5]decan-2-yl)ethoxy)-2-methylphenyl)picolinate

A mixture of (r)-2-(2-(2-(3-(6-(8-(benzo[d]thiazol-2-ylcarbamoyl)-3,4-dihydroisoquinolin-2(1 h)-yl)-2-(tert-butoxycarbonyl)pyridin-3-yl)-2-methylphenoxy)ethyl)-8-azaspiro[4.5]decan-8-yl)acetic acid (80.00 mg, 98.04 μmol, 1 equiv.), 3-(6-amino-1-methyl-1H-indazol-3-yl)piperidine-2,6-dione (30.38 mg, 117.64 μmol, 1.2 equiv.), and EDCI (28.19 mg, 147.06 μmol, 1.5 equiv.) in pyridine (1 mL) was degassed and purged with N₂ three times, and then the mixture was stirred at 25° C. for 2 hours under N₂ atmosphere. The reaction mixture was filtered and concentrated under reduced pressure to give a residue. The residue was used in the next step without further purification. The compound tert-butyl 6-(8-(benzo[d]thiazol-2-ylcarbamoyl)-3,4-dihydroisoquinolin-2(1H)-yl)-3-(3-(2-((2R)-8-(2-((3-(2,6-dioxopiperidin-3-yl)-1-methyl-1H-indazol-6-yl)amino)-2-oxoethyl)-8-azaspiro[4.5]decan-2-yl)ethoxy)-2-methylphenyl)picolinate (80 mg, 75.7 μmol, 77.2% yield) was obtained as a colorless oil.

MS (ESI) m/z: 1056.4[M+H]⁺

### Step D. Procedure for Preparation of 6-(8-(benzo[d]thiazol-2-ylcarbamoyl)-3,4-dihydro-isoquinolin-2(1 h)-yl)-3-(3-(2-((2r)-8-(2-((3-(2,6-dioxopiperidin-3-yl)-1-methyl-1 h-indazol-6-yl)amino)-2-oxoethyl)-8-azaspiro[4.5]decan-2-yl)ethoxy)-2-methylphenyl)picolinic acid [rel-6-(8-(benzo[d]thiazol-2-ylcarbamoyl)-3,4-dihydroisoquinolin-2(1H)-yl)-3-(3-(2-((2R)-8-(2-((3-(2,6-dioxopiperidin-3-yl)-1-methyl-1H-indazol-6-yl)amino)-2-oxoethyl)-8-azaspiro[4.5]decan-2-yl)ethoxy)-2-methylphenyl)picolinic acid]

A mixture of tert-butyl 6-(8-(benzo[d]thiazol-2-ylcarbamoyl)-3,4-dihydroisoquinolin-2(1H)-yl)-3-(3-(2-((2R)-8-(2-((3-(2,6-dioxopiperidin-3-yl)-1-methyl-1H-indazol-6-yl)amino)-2-oxoethyl)-8-azaspiro[4.5]decan-2-yl)ethoxy)-2-methylphenyl)picolinate (80 mg, 75.74 μmol, 1 equiv.) in DCM (0.8 mL) and TFA (0.8 mL) was degassed and purged with N₂ three times, and then the mixture was stirred at 25° C. for 16 hours. The reaction mixture was filtered and concentrated under reduced pressure to give a residue. The residue was purified by prep-HPLC. The compound 6-(8-(benzo[d]thiazol-2-ylcarbamoyl)-3,4-dihydro-isoquinolin-2(1 h)-yl)-3-(3-(2-((2r)-8-(2-((3-(2,6-dioxopiperidin-3-yl)-1-methyl-1 h-indazol-6-yl)amino)-2-oxoethyl)-8-azaspiro[4.5]decan-2-yl)ethoxy)-2-methylphenyl)picolinic acid [rel-6-(8-(benzo[d]thiazol-2-ylcarbamoyl)-3,4-dihydroisoquinolin-2(1H)-yl)-3-(3-(2-((2R)-8-(2-((3-(2,6-dioxopiperidin-3-yl)-1-methyl-1H-indazol-6-yl)amino)-2-oxoethyl)-8-azaspiro[4.5]decan-2-yl)ethoxy)-2-methylphenyl)picolinic acid] (22.69 mg, 22.5 μmol, 29.7% yield, 99.1% purity) was obtained as a yellow solid.

MS (ESI) m/z: 1000.4 [M+H]⁺.

¹H NMR (400 MHz, DMSO-d₆) δ=12.91-12.81 (m, 1H), 10.88 (s, 1H), 9.89-9.75 (m, 1H), 8.02 (d, J=7.2 Hz, 2H), 7.79 (d, J=8.0 Hz, 1H), 7.66-7.60 (m, 2H), 7.49-7.42 (m, 3H), 7.40-7.32 (m, 2H), 7.24-7.17 (m, 1H), 7.09 (t, J=8.0 Hz, 1H), 6.97 (d, J=9.2 Hz, 1H), 6.89 (d, J=8.0 Hz, 1H), 6.63 (d, J=8.0 Hz, 1H), 4.98 (s, 2H), 4.32 (dd, J=5.2, 9.6 Hz, 1H), 3.99-3.89 (m, 7H), 3.30-3.28 (m, 4H), 3.05-3.00 (m, 2H), 2.67-2.58 (m, 4H), 2.37-2.31 (m, 2H), 1.91-1.76 (m, 7H), 1.42-1.42 (m, 1H), 1.64-1.42 (m, 5H), 1.35-1.19 (m, 1H), 1.19-1.18 (m, 1H), 1.10-1.03 (m, 1H)

### Example 207. Preparation of Compound 281a

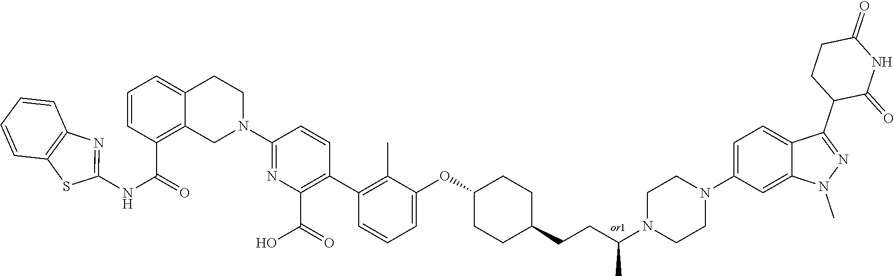

### 6-(8-(benzo[d]thiazol-2-ylcarbamoyl)-3,4-dihydroisoquinolin-2(1H)-yl)-3-(3-(((1R,4r)-4-((3S)-3-(4-(3-(2,6-dioxopiperidin-3-yl)-1-methyl-1H-indazol-6-yl)piperazin-1-yl)butyl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1R,4r)-4-((3S*)-3-(4-(3-(2,6-dioxopiperidin-3-yl)-1-methyl-1H-indazol-6-yl)piperazin-1-yl)butyl)cyclohexyl)oxy)-2-methylphenyl)picolinic acid]

### Step A. Procedure for Preparation of ((1r,4r)-4-(3-bromo-2-methylphenoxy)cyclohexyl)methanol

To a mixture of methyl (1r,4r)-4-(3-bromo-2-methylphenoxy)cyclohexane-1-carboxylate (40 g, 48.90 mmol, 40% purity, 1 equiv.) in THE (350 mL) was slowly added LAH (1.95 g, 51.34 mmol, 1.05 equiv.) at 0° C. and then the mixture was stirred at 25° C. for 2 hours. The mixture was quenched by Na₂SO₄·10 H₂O (3 g). The solution was slowly poured into ice-water (300 mL) and the pH was acidized to pH 4˜5 with 1 M HCl, extracted with EtOAc 900 mL (300 mL×3), washed with brine (400 mL), dried by sodium sulfate, filtered, and concentrated under reduced pressure. The residue was purified by flash silica gel chromatography (eluent of 0˜12% ethyl acetate/petroleum ether) to give ((1r,4r)-4-(3-bromo-2-methylphenoxy)cyclohexyl)methanol (20.5 g, 67.0 mmol, 45.6% yield) as a white solid.

¹H NMR (400 MHz, DMSO-d₆) δ=7.15-7.11 (m, 1H), 7.06 (t, J=8.0 Hz, 1H), 7.03-6.99 (m, 1H), 4.26-4.17 (m, 1H), 2.21 (s, 3H), 2.06 (dd, J=2.4, 11.6 Hz, 2H), 1.98 (s, 1H), 1.78 (d, J=11.6 Hz, 2H), 1.58 (d, J=4.8 Hz, 2H), 1.40-1.34 (m, 5H)

### Step B. Procedure for Preparation of (1s,4s)-4-(3-bromo-2-methylphenoxy)cyclohexane-1-carbaldehyde

To a mixture of (COCl)₂ (8.48 g, 66.84 mmol, 5.85 mL, 2 equiv.) in DCM (80 mL) at −78° C. was added DMSO (10.45 g, 133.69 mmol, 10.45 mL, 4 equiv.) and the mixture was stirred for 0.5 hours. To the solution was added ((1r,4r)-4-(3-bromo-2-methylphenoxy)cyclohexyl)methanol (10 g, 33.42 mmol, 1 equiv.) in DCM (200 mL) and the mixture was stirred at −78° C. for 1 hour. Then to the mixture was added TEA (20.29 g, 200.53 mmol, 27.91 mL, 6 equiv.) and the mixture was warmed to 25° C. and stirred for 0.5 hour under N₂ atmosphere. The reaction mixture was diluted with H₂O (150 mL) and extracted with DCM 300 mL (100 mL×3). The combined organic layers were washed with saturated NaCl (200 mL), filtered and concentrated under reduced pressure to give (1s,4s)-4-(3-bromo-2-methylphenoxy)cyclohexane-1-carbaldehyde (33.2 g, crude) as a white solid.

MS (ESI) m/z: 296.9 [M+H]⁺.

¹H NMR (400 MHz, DMSO-d₆) δ=9.61 (s, 1H), 7.16-7.13 (m, 1H), 7.10-7.05 (m, 1H), 7.04-7.01 (m, 1H), 4.34-4.27 (m, 1H), 2.39-2.33 (m, 1H), 2.25-2.20 (m, 3H), 2.03-1.93 (m, 4H), 1.60-1.55 (m, 2H), 1.46 (d, J=9.6 Hz, 2H)

### Step C. Procedure for Preparation of ethyl (E)-3-((1s,4s)-4-(3-bromo-2-methylphenoxy)cyclohexyl)acrylate

A mixture of NaH (4.95 g, 123.83 mmol, 60% purity, 1.6 equiv.) in THE (200 mL) was purged with N₂ three times and ethyl 2-(diethoxyphosphoryl)acetate (24.29 g, 108.35 mmol, 21.50 mL, 1.4 equiv.) was slowly added at 0° C., and the mixture was stirred for 1.5 hours under N₂ atmosphere. To the solution was added (1s,4s)-4-(3-bromo-2-methylphenoxy)cyclohexane-1-carbaldehyde (23 g, 77.39 mmol, 1 equiv.) at 0° C., the mixture was warmed to 25° C., and stirred for 1.5 hours under N₂ atmosphere. The reaction mixture was quenched by addition of saturated NH₄Cl (150 mL) under 0° C. The mixture was extracted with DCM 300 mL (100 mL×3). The combined organic layers were washed with brine 200 mL (100 mL×2), dried over sodium sulfate, filtered, and concentrated under reduced pressure to give a residue. The residue was purified by flash silica gel chromatography (eluent of 0˜13% ethyl acetate/petroleum ether) to give ethyl (E)-3-((1s,4s)-4-(3-bromo-2-methylphenoxy)cyclohexyl)acrylate (11.3 g, 30.7 mmol, 39.7% yield) as a colorless oil.

### Step D. Procedure for Preparation of ethyl 3-((1r,4r)-4-(3-bromo-2-methylphenoxy)cyclohexyl)propanoate

A mixture of ethyl (E)-3-((1s,4s)-4-(3-bromo-2-methylphenoxy)cyclohexyl)acrylate (11 g, 29.95 mmol, 1 equiv.) and PtO₂ (3.40 g, 14.98 mmol, 0.5 equiv.) in EtOH (200 mL) was degassed and purged with H₂ three times, and then the mixture was stirred at 20° C. for 23 hours under H₂ atmosphere. The mixture was filtered by EtOH (50 mL) and concentrated under reduced pressure to give ethyl 3-((1r,4r)-4-(3-bromo-2-methylphenoxy)cyclohexyl)propanoate (9.1 g, 20.1 mmol, 67.3% yield, 81.8% purity) as a gray oil.

¹H NMR (400 MHz, DMSO-d₆) δ=7.15-7.11 (m, 1H), 7.06 (t, J=8.0 Hz, 1H), 7.02-6.99 (m, 1H), 4.26-4.17 (m, 1H), 4.05 (q, J=7.2 Hz, 2H), 2.29 (t, J=7.6 Hz, 2H), 2.20 (s, 3H), 2.03 (dd, J=2.8, 12.4 Hz, 2H), 1.78-1.71 (m, 2H), 1.46 (q, J=7.2 Hz, 2H), 1.38-1.29 (m, 2H), 1.27-1.20 (m, 1H), 1.17 (t, J=7.2 Hz, 3H), 1.10-0.99 (m, 2H)

### Step E. Procedure for Preparation of 3-((1r,4r)-4-(3-bromo-2-methylphenoxy)cyclohexyl)propanoic acid

A mixture of ethyl 3-((1r,4r)-4-(3-bromo-2-methylphenoxy)cyclohexyl)propanoate (3 g, 8.12 mmol, 1 equiv.) and LiOH·H₂O (1 M, 8.12 mL, 1 equiv.) in THE (30 mL) was stirred at 25° C. for 18 hours. The mixture was concentrated and diluted with H₂O (20 mL), then the pH of the mixture was adjusted to 3 with 1 M HCl. Then the mixture was filtered and concentrated to give 3-((1r,4r)-4-(3-bromo-2-methylphenoxy)cyclohexyl)propanoic acid (2.5 g, 6.8 mmol, 83.6% yield, 92.4% purity) as a colorless oil.

¹H NMR (400 MHz, DMSO-d₆) δ=12.27-11.77 (m, 1H), 7.15-7.11 (m, 1H), 7.06 (t, J=8.0 Hz, 1H), 7.02-6.99 (m, 1H), 4.26-4.17 (m, 1H), 2.25-2.21 (m, 2H), 2.20 (s, 3H), 2.07-2.00 (m, 2H), 1.75 (d, J=11.6 Hz, 2H), 1.43 (q, J=7.2 Hz, 2H), 1.38-1.30 (m, 2H), 1.25 (td, J=3.6, 7.2 Hz, 1H), 1.09-0.98 (m, 2H)

### Step F. Procedure for Preparation of 3-((1r,4r)-4-(3-bromo-2-methylphenoxy)cyclohexyl)-N-methoxy-N-methylpropanamide

A mixture of 3-((1r,4r)-4-(3-bromo-2-methylphenoxy)cyclohexyl)propanoic acid (2.4 g, 7.03 mmol, 1 equiv.) and N,O-dimethylhydroxylamine (1.29 g, 21.10 mmol, 3 equiv.) in THE (24 mL) was added HATU (8.02 g, 21.10 mmol, 3 equiv.) and TEA (2.14 g, 21.10 mmol, 2.94 mL, 3 equiv.) and stirred at 25° C. for 18 hours. The reaction mixture was filtered and concentrated under reduced pressure to give a residue. The residue was purified by flash silica gel chromatography (eluent of 0˜26% ethyl acetate/petroleum ether) to give 3-((1r,4r)-4-(3-bromo-2-methylphenoxy)cyclohexyl)-N-methoxy-N-methylpropanamide (2.5 g, 6.4 mmol, 91.8% yield, 99.2% purity) as a colorless oil.

MS (ESI) m/z: 384.0 [M+H]⁺

¹H NMR (400 MHz, DMSO-d₆) δ=7.15-7.12 (m, 1H), 7.06 (t, J=8.0 Hz, 1H), 7.03-7.00 (m, 1H), 4.22 (t, J=4.0 Hz, 1H), 3.65 (s, 3H), 3.07 (s, 3H), 2.38 (t, J=7.6 Hz, 2H), 2.20 (s, 3H), 2.08-2.00 (m, 2H), 1.77 (d, J=11.6 Hz, 2H), 1.46-1.39 (m, 2H), 1.39-1.30 (m, 2H), 1.30-1.25 (m, 1H), 1.11-0.99 (m, 2H)

### Step G. Procedure for Preparation of 4-((1r,4r)-4-(3-bromophenoxy)cyclohexyl)butan-2-one

To a solution of 3-((1r,4r)-4-(3-bromo-2-methylphenoxy)cyclohexyl)-N-methoxy-N-methylpropanamide (2.3 g, 5.98 mmol, 1 equiv.) in THE (23 mL) was added bromo(methyl)magnesium (3 M, 3.99 mL, 2 equiv.) at −78° C. The mixture was stirred at 25° C. for 18 hours. The reaction mixture was quenched by addition of saturated NH₄Cl (25 mL) at 0° C. The mixture was extracted with DCM 90 mL (30 mL×3). The combined organic layers were washed with brine 60 mL (30 mL×2), dried over sodium sulfate, filtered, and concentrated under reduced pressure to give 4-((1r,4r)-4-(3-bromophenoxy)cyclohexyl)butan-2-one (2.05 g, crude) as a colorless oil.

¹H NMR (400 MHz, DMSO-d₆) δ=7.16-7.12 (m, 1H), 7.06 (t, J=8.0 Hz, 1H), 7.03-7.00 (m, 1H), 4.27-4.17 (m, 1H), 2.44 (t, J=7.6 Hz, 2H), 2.20 (s, 3H), 2.08 (s, 3H), 2.03 (d, J=9.6 Hz, 2H), 1.77-1.71 (m, 2H), 1.41-1.36 (m, 2H), 1.35-1.27 (m, 2H), 1.22-1.15 (m, 1H), 1.09-0.99 (m, 2H)

### Step H. Procedure for Preparation of 4-((1r,4s)-4-(3-bromo-2-methylphenoxy)cyclohexyl)butan-2-ol

To a mixture of 4-((1r,4r)-4-(3-bromophenoxy)cyclohexyl)butan-2-one (1.97 g, 5.81 mmol, 1 equiv.) in THF (20 mL) was slowly added LAH (220.50 mg, 5.81 mmol, 1 equiv.) at 0° C., and then the mixture was stirred at 25° C. for 2 hours. The mixture was quenched by 3 g Na₂SO₄·10H₂O. The solution was slowly poured into ice-water (50 mL) and the pH was acidized to pH 4-5 with 1 M HCl, extracted with EtOAc 150 mL (50 mL×3), washed with brine 80 mL, dried by sodium sulfate, filtered and concentrated under reduced pressure to give 4-((1r,4s)-4-(3-bromo-2-methylphenoxy)cyclohexyl)butan-2-ol (1.7 g, crude) as a colorless oil.

### Step I. Procedure for preparation of (R)-4-((1r,4S)-4-(3-bromo-2-methylphenoxy)cyclohexyl)butan-2-ol

### 4-((1r,4s)-4-(3-bromo-2-methylphenoxy)cyclohexyl)butan-2-ol was purified by prep-HPLC to give (S)-4-((1r,4R)-4-(3-bromo-2-methylphenoxy)cyclohexyl)butan-2-ol and (R)-4-((1r,4S)-4-(3-bromo-2-methylphenoxy)cyclohexyl)butan-2-ol

¹H NMR (400 MHz, DMSO-d₆) δ=7.16-7.11 (m, 1H), 7.06 (t, J=8.0 Hz, 1H), 7.03-6.99 (m, 1H), 4.33 (s, 1H), 4.26-4.18 (m, 1H), 3.56-3.50 (m, 1H), 2.20 (s, 3H), 2.03 (d, J=10.8 Hz, 2H), 1.75 (d, J=12.4 Hz, 2H), 1.41-1.25 (m, 6H), 1.21-1.16 (m, 1H), 1.08-0.98 (m, 5H)

¹H NMR (400 MHz, DMSO-d₆) δ=7.14-7.11 (m, 1H), 7.06 (t, J=8.0 Hz, 1H), 7.02-6.98 (m, 1H), 4.35 (d, J=4.8 Hz, 1H), 4.26-4.16 (m, 1H), 3.53 (td, J=5.5, 11.1 Hz, 1H), 2.20 (s, 3H), 2.03 (d, J=10.0 Hz, 2H), 1.75 (d, J=12.4 Hz, 2H), 1.39-1.23 (m, 6H), 1.19-1.15 (m, 1H), 1.06-0.98 (m, 5H)

### Step J. Procedure for Preparation of (R)-4-((1r,4S)-4-(3-bromo-2-methylphenoxy)cyclohexyl)butan-2-yl 4-methylbenzenesulfonate

To a mixture of (R)-4-((1r,4S)-4-(3-bromo-2-methylphenoxy)cyclohexyl)butan-2-ol (360 mg, 1.05 mmol, 1 equiv.), TEA (320.22 mg, 3.16 mmol, 440.46 μL, 3 equiv.), and DMAP (19.33 mg, 158.23 μmol, 0.15 equiv.) in DCM (5 mL) was added TsCl (301.65 mg, 1.58 mmol, 1.5 equiv.) at 0° C., and then the mixture was stirred at 40° C. for 3 hours. The reaction mixture was concentrated under reduced pressure to give a residue. The residue was purified by flash silica gel chromatography (eluent of 0˜17% ethyl acetate/petroleum ether) to give (R)-4-((1r,4S)-4-(3-bromo-2-methylphenoxy)cyclohexyl)butan-2-yl 4-methylbenzenesulfonate (488 mg, 984.9 μmol, 93.3% yield) as a yellow solid.

### Step K. Procedure for Preparation of 3-(6-(4-((S)-4-((1r,4R)-4-(3-bromo-2-methylphenoxy)cyclohexyl)butan-2-yl)piperazin-1-yl)-1-methyl-1H-indazol-3-yl)piperidine-2,6-dione

To a solution of (R)-4-((1r,4S)-4-(3-bromo-2-methylphenoxy)cyclohexyl)butan-2-yl 4-methylbenzenesulfonate (200 mg, 403.66 μmol, 1 equiv.) in ACN (4 mL) was added K₂CO₃ (167.36 mg, 1.21 mmol, 3 equiv.), KI (67.01 mg, 403.66 μmol, 1 equiv.), and 3-(1-methyl-6-piperazin-1-yl-indazol-3-yl)piperidine-2,6-dione (161.55 mg, 444.02 μmol, 1.1 equiv., HCl). The mixture was stirred at 60° C. for 10 hours. The reaction mixture was concentrated under reduced pressure to give a residue. The residue was purified by column chromatography (SiO₂, petroleum ether:ethyl acetate=10/1 to 0/1) to give 3-(6-(4-((S)-4-((1r,4R)-4-(3-bromo-2-methylphenoxy)cyclohexyl)butan-2-yl)piperazin-1-yl)-1-methyl-1H-indazol-3-yl)piperidine-2,6-dione (120 mg, 184.4 μmol, 45.6% yield) as a yellow oil.

MS (ESI) m/z: 682.4 [M+H]⁺.

### Step L. Procedure for Preparation of tert-butyl 6-(8-(benzo[d]thiazol-2-ylcarbamoyl)-3,4-dihydroisoquinolin-2(1H)-yl)-3-(3-(((1R,4r)-4-((3S)-3-(4-(3-(2,6-dioxopiperidin-3-yl)-1-methyl-1H-indazol-6-yl)piperazin-1-yl)butyl)cyclohexyl)oxy)-2-methylphenyl)picolinate

A mixture of tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (100 mg, 163.25 μmol, 1 eq), 3-(6-(4-((S)-4-((1r,4R)-4-(3-bromo-2-methylphenoxy)cyclohexyl)butan-2-yl)piperazin-1-yl)-1-methyl-1H-indazol-3-yl)piperidine-2,6-dione (106.22 mg, 163.25 μmol, 1 equiv.), [2-(2-aminophenyl)phenyl]palladium(1+); bis(1-adamantyl)-butyl-phosphane; methanesulfonate (11.89 mg, 16.33 μmol, 0.1 equiv.), and KF (28.46 mg, 489.76 μmol, 11.47 μL, 3 equiv.) in dioxane (4 mL) and H₂O (1 mL) was degassed and purged with N₂ three times and then the mixture was stirred at 100° C. for 1 hour under N₂ atmosphere. The reaction mixture was filtered and concentrated under reduced pressure to give a residue. The residue was purified by column chromatography (SiO₂, dichloromethane:methanol=100/1 to 30/1) to give tert-butyl 6-(8-(benzo[d]thiazol-2-ylcarbamoyl)-3,4-dihydroisoquinolin-2(1H)-yl)-3-(3-(((1R,4r)-4-((3S)-3-(4-(3-(2,6-dioxopiperidin-3-yl)-1-methyl-1H-indazol-6-yl)piperazin-1-yl)butyl)cyclohexyl)oxy)-2-methylphenyl)picolinate (110 mg, 104.1 μmol, 63.7% yield) as a yellow solid.

MS (ESI) m/z: 1056.5 [M+H]⁺.

### Step M. Procedure for Preparation of 6-(8-(benzo[d]thiazol-2-ylcarbamoyl)-3,4-dihydroisoquinolin-2(1H)-yl)-3-(3-(((1R,4r)-4-((3S)-3-(4-(3-(2,6-dioxopiperidin-3-yl)-1-methyl-1H-indazol-6-yl)piperazin-1-yl)butyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1R,4r)-4-((3S*)-3-(4-(3-(2,6-dioxopiperidin-3-yl)-1-methyl-1H-indazol-6-yl)piperazin-1-yl)butyl)cyclohexyl)oxy)-2-methylphenyl)picolinic acid]

To a solution of tert-butyl 6-(8-(benzo[d]thiazol-2-ylcarbamoyl)-3,4-dihydroisoquinolin-2(1H)-yl)-3-(3-(((1R,4r)-4-((3S)-3-(4-(3-(2,6-dioxopiperidin-3-yl)-1-methyl-1H-indazol-6-yl)piperazin-1-yl)butyl)cyclohexyl)oxy)-2-methylphenyl)picolinate (100.09 mg, 94.76 μmol, 1 equiv.) in DCM (1 mL) was added TFA (1.54 g, 13.51 mmol, 1 mL, 142.54 equiv.). The mixture was stirred at 40° C. for 1 hour. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1R,4r)-4-((3S)-3-(4-(3-(2,6-dioxopiperidin-3-yl)-1-methyl-1H-indazol-6-yl)piperazin-1-yl)butyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1R,4r)-4-((3S*)-3-(4-(3-(2,6-dioxopiperidin-3-yl)-1-methyl-1H-indazol-6-yl)piperazin-1-yl)butyl)cyclohexyl)oxy)-2-methylphenyl)picolinic acid] (16.5 mg, 15.1 μmol, 16.0% yield, 95.6% purity) as a yellow solid.

MS (ESI) m/z: 1000.4 [M+H]⁺.

¹H NMR (400 MHz, DMSO-d₆) δ=10.85 (s, 1H), 8.15 (s, 1H), 8.04 (d, J=7.6 Hz, 1H), 7.79 (d, J=8.0 Hz, 1H), 7.63 (d, J=7.2 Hz, 1H), 7.52-7.43 (m, 4H), 7.41-7.32 (m, 2H), 7.10-7.04 (m, 1H), 6.98-6.90 (m, 3H), 6.84 (s, 1H), 6.62 (d, J=7.2 Hz, 1H), 4.98 (s, 2H), 4.28-4.24 (m, 1H), 4.20 (t, J=10.8 Hz, 1H), 3.92 (s, 2H), 3.89 (s, 3H), 3.21 (s, 4H), 3.03 (t, J=5.2 Hz, 2H), 2.68 (s, 2H), 2.63-2.57 (m, 5H), 2.32-2.25 (m, 1H), 2.20-2.13 (m, 1H), 2.11-2.05 (m, 2H), 1.87 (s, 3H), 1.83-1.77 (m, 2H), 1.58-1.50 (m, 1H), 1.40-1.33 (m, 2H), 1.31-1.22 (m, 4H), 1.12-1.04 (m, 2H), 0.98 (d, J=6.4 Hz, 3H)

### Example 208. Preparation of Compound 281b

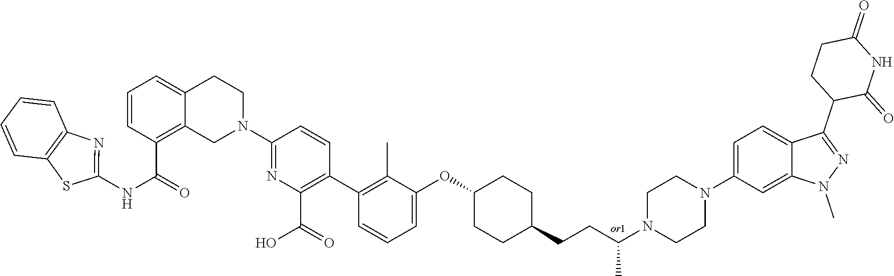 [SMILES: Cc1c(O[C@H]2CC[C@H](CC[C@@H](C)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1S,4r)-4-((3R)-3-(4-(3-(2,6-dioxopiperidin-3-yl)-1-methyl-1H-indazol-6-yl)piperazin-1-yl)butyl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1S,4r)-4-((3R*)-3-(4-(3-(2,6-dioxopiperidin-3-yl)-1-methyl-1H-indazol-6-yl)piperazin-1-yl)butyl)cyclohexyl)oxy)-2-methylphenyl)picolinic acid]

### Step A. Procedure for Preparation of [(1S)-3-[4-(3-bromo-2-methyl-phenoxy)cyclohexyl]-1-methyl-propyl] 4-methylbenzenesulfonate

To a solution of (2S)-4-[4-(3-bromo-2-methyl-phenoxy)cyclohexyl]butan-2-ol (300 mg, 879.04 μmol, 1 equiv.) in DCM (3 mL) was added 4-methylbenzenesulfonyl chloride (184.34 mg, 966.94 μmol, 1.1 equiv.), TEA (444.75 mg, 4.40 mmol, 611.76 μL, 5 equiv.), and DMAP (10.74 mg, 87.90 μmol, 0.1 equiv.) at 25° C. The reaction mixture was stirred at 60° C. for 16 hours. The reaction mixture was filtered and concentrated under reduced pressure to give a residue. The residue was purified by flash silica gel chromatography (eluent of 0˜30% ethyl acetate/petroleum ether). The compound [(1S)-3-[4-(3-bromo-2-methyl-phenoxy)cyclohexyl]-1-methyl-propyl] 4-methylbenzenesulfonate (167 mg, 322.2 μmol, 36.6% yield, 95.6% purity) was obtained as a yellow oil.

MS (ESI) m/z: 517.1 [M+Na]⁺.

¹H NMR (400 MHz, CDCl₃-d) δ=7.81 (d, J=8.4 Hz, 2H), 7.35 (d, J=8.0 Hz, 2H), 7.14 (d, J=8.0 Hz, 1H), 6.97 (t, J=8.0 Hz, 1H), 6.79 (d, J=8.0 Hz, 1H), 4.65-4.56 (m, 1H), 4.10-3.99 (m, 1H), 2.46 (s, 3H), 2.29 (s, 3H), 2.09 (dd, J=2.8, 12.4 Hz, 2H), 1.78-1.67 (m, 2H), 1.66-1.60 (m, 1H), 1.55-1.49 (m, 1H), 1.45-1.33 (m, 2H), 1.26 (d, J=6.3 Hz, 3H), 1.22-1.10 (m, 3H), 1.00-0.87 (m, 2H)

### Step B. Procedure for Preparation of 3-(6-(4-((R)-4-((1r,4S)-4-(3-bromo-2-methylphenoxy)cyclohexyl)butan-2-yl)piperazin-1-yl)-1-methyl-1H-indazol-3-yl)piperidine-2,6-dione

To a solution of [(1S)-3-[4-(3-bromo-2-methyl-phenoxy)cyclohexyl]-1-methyl-propyl] 4-methylbenzenesulfonate (80 mg, 161.46 μmol, 1 equiv.) in CH₃CN (1 mL) was added 3-(1-methyl-6-piperazin-1-yl-indazol-3-yl)piperidine-2,6-dione (63.43 mg, 193.76 μmol, 1.2 equiv.), K₂CO₃ (66.95 mg, 484.39 μmol, 3 equiv.), and KI (26.80 mg, 161.46 μmol, 1 equiv.) at 25° C. The reaction was stirred at 60° C. for 16 hours. The reaction mixture was filtered and concentrated under reduced pressure to give a residue. The residue was purified by flash silica gel chromatography (eluent of 0˜10% ethyl acetate/petroleum ether). The compound 3-(6-(4-((R)-4-((1r,4S)-4-(3-bromo-2-methylphenoxy)cyclohexyl)butan-2-yl)piperazin-1-yl)-1-methyl-1H-indazol-3-yl)piperidine-2,6-dione (36 mg, 51.8 μmol, 32.1% yield, 93.7% purity) was obtained as a yellow oil.

### Step C. Procedure for Preparation of tert-butyl 6-(8-(benzo[d]thiazol-2-ylcarbamoyl)-3,4-dihydroisoquinolin-2(1H)-yl)-3-(3-((4-((3R)-3-(4-(3-(2,6-dioxopiperidin-3-yl)-1-methyl-1H-indazol-6-yl)piperazin-1-yl)butyl)cyclohexyl)oxy)-2-methylphenyl)picolinate

tert-Butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (37.28 mg, 60.86 μmol, 1.1 equiv.), 3-(6-(4-((R)-4-((1r,4S)-4-(3-bromo-2-methylphenoxy)cyclohexyl)butan-2-yl)piperazin-1-yl)-1-methyl-1H-indazol-3-yl)piperidine-2,6-dione (36 mg, 55.33 μmol, 1 equiv.), KF (9.64 mg, 165.99 mol, 3.89 μL, 3 equiv.), and [2-(2-aminophenyl)phenyl]palladium(1+); bis(1-adamantyl)-butyl-phosphane; methanesulfonate (8.06 mg, 11.07 μmol, 0.2 equiv.) were taken up into a microwave tube in dioxane (2 mL) and H₂O (0.2 mL). The sealed tube was heated at 100° C. for 1 hour under microwave. The reaction mixture was filtered and concentrated under reduced pressure to give a residue. The residue was purified by flash silica gel chromatography (eluent of 0˜50% ethyl acetate/petroleum ether). The compound tert-butyl 6-(8-(benzo[d]thiazol-2-ylcarbamoyl)-3,4-dihydroisoquinolin-2(1H)-yl)-3-(3-((4-((3R)-3-(4-(3-(2,6-dioxopiperidin-3-yl)-1-methyl-1H-indazol-6-yl)piperazin-1-yl)butyl)cyclohexyl)oxy)-2-methylphenyl)picolinate (69 mg, 29.4 μmol, 53.1% yield, 45% purity) was obtained as a yellow solid.

MS (ESI) m/z: 1156.4 [M+H]⁺.

### Step D. Procedure for Preparation of 6-(8-(benzo[d]thiazol-2-ylcarbamoyl)-3,4-dihydroisoquinolin-2(1H)-yl)-3-(3-(((1S,4r)-4-((3R)-3-(4-(3-(2,6-dioxopiperidin-3-yl)-1-methyl-1H-indazol-6-yl)piperazin-1-yl)butyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1S,4r)-4-((3R*)-3-(4-(3-(2,6-dioxopiperidin-3-yl)-1-methyl-1H-indazol-6-yl)piperazin-1-yl)butyl)cyclohexyl)oxy)-2-methylphenyl)picolinic acid]

A solution of tert-butyl 6-(8-(benzo[d]thiazol-2-ylcarbamoyl)-3,4-dihydroisoquinolin-2(1H)-yl)-3-(3-((4-((3R)-3-(4-(3-(2,6-dioxopiperidin-3-yl)-1-methyl-1H-indazol-6-yl)piperazin-1-yl)butyl)cyclohexyl)oxy)-2-methylphenyl)picolinate (69 mg, 29.42 μmol, 45% purity, 1 equiv.) in DCM (0.5 mL) and TFA (0.5 mL) was stirred at 25° C. for 16 hours. The reaction mixture was filtered and concentrated under reduced pressure to give a residue. The residue was purified by prep-HPLC. The compound 6-(8-(benzo[d]thiazol-2-ylcarbamoyl)-3,4-dihydroisoquinolin-2(1H)-yl)-3-(3-(((1S,4r)-4-((3R)-3-(4-(3-(2,6-dioxopiperidin-3-yl)-1-methyl-1H-indazol-6-yl)piperazin-1-yl)butyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1S,4r)-4-((3R*)-3-(4-(3-(2,6-dioxopiperidin-3-yl)-1-methyl-1H-indazol-6-yl)piperazin-1-yl)butyl)cyclohexyl)oxy)-2-methylphenyl)picolinic acid] (13.0 mg, 12.2 μmol, 41.5% yield, 93.6% purity) was obtained as a yellow solid.

MS (ESI) m/z: 1000.4 [M+H]⁺.

¹H NMR (400 MHz, DMSO-d₆) δ=12.95-12.39 (m, 2H), 10.85 (s, 1H), 8.03 (d, J=8.0 Hz, 1H), 7.79 (d, J=8.4 Hz, 1H), 7.65-7.60 (m, 1H), 7.55 (d, J=8.4 Hz, 1H), 7.49-7.42 (m, 3H), 7.40-7.32 (m, 2H), 7.08 (t, J=8.0 Hz, 1H), 7.00-6.90 (m, 4H), 6.62 (d, J=7.6 Hz, 1H), 4.98 (s, 2H), 4.30-4.17 (m, 2H), 3.91 (s, 6H), 3.03 (t, J=5.6 Hz, 2H), 2.69-2.56 (m, 5H), 2.36-2.26 (m, 2H), 2.22-2.04 (m, 4H), 1.87 (s, 3H), 1.85-1.78 (m, 2H), 1.59-1.00 (m, 14H)

### Example 209. Preparation of Compound 283a

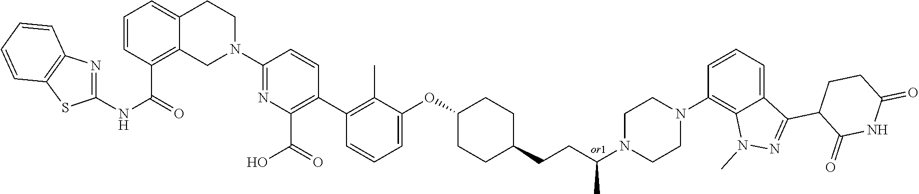 [SMILES: Cc1c(O[C@H]2CC[C@H](CC[C@H](C)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1R,4r)-4-((3S)-3-(4-(3-(2,6-dioxopiperidin-3-yl)-1-methyl-1H-indazol-7-yl)piperazin-1-yl)butyl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1R,4r)-4-((3S*)-3-(4-(3-(2,6-dioxopiperidin-3-yl)-1-methyl-1H-indazol-7-yl)piperazin-1-yl)butyl)cyclohexyl)oxy)-2-methylphenyl)picolinic acid]

### Step A. Procedure for Preparation of 3-(7-(4-((S)-4-((1r,4R)-4-(3-bromo-2-methylphenoxy)cyclohexyl)butan-2-yl)piperazin-1-yl)-1-methyl-1H-indazol-3-yl)piperidine-2,6-dione

To a solution of [(1R)-3-[4-(3-bromo-2-methyl-phenoxy)cyclohexyl]-1-methyl-propyl] 4-methylbenzenesulfonate (200 mg, 403.66 μmol, 1 equiv.) in CH₃CN (4 mL) was added K₂CO₃ (167.36 mg, 1.21 mmol, 3 equiv.), KI (67.01 mg, 403.66 μmol, 1 equiv.), and 3-(1-methyl-7-piperazin-1-yl-indazol-3-yl)piperidine-2,6-dione (161.55 mg, 444.03 μmol, 1.1 equiv., HCl). The mixture was stirred at 60° C. for 10 hours. The reaction mixture was concentrated under reduced pressure to give a residue. The residue was purified by column chromatography (SiO₂, petroleum ether:ethyl acetate=10/1 to 0/1) to give 3-(7-(4-((S)-4-((1r,4R)-4-(3-bromo-2-methylphenoxy)cyclohexyl)butan-2-yl)piperazin-1-yl)-1-methyl-1H-indazol-3-yl)piperidine-2,6-dione (120 mg, 184.4 μmol, 45.6% yield) was obtained as a yellow oil.

MS (ESI) m/z: 650.3 [M+H]⁺.

### Step B. Procedure for Preparation of tert-butyl 6-[8-(1,3-benzothiazol-2-ylcarbamoyl)-3,4-dihydro-1H-isoquinolin-2-yl]-3-[3-[4-[(3S)-3-[4-[3-(2,6-dioxo-3-piperidyl)-1-methyl-indazol-7-yl]piperazin-1-yl]butyl]cyclohexoxy]-2-methyl-phenyl]pyridine-2-carboxylate

A mixture of tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (110 mg, 179.58 μmol, 1 equiv.), 3-(7-(4-((S)-4-((1r,4R)-4-(3-bromo-2-methylphenoxy)cyclohexyl)butan-2-yl)piperazin-1-yl)-1-methyl-1H-indazol-3-yl)piperidine-2,6-dione (116.84 mg, 179.58 μmol, 1 equiv.), [2-(2-aminophenyl)phenyl]palladium(1+); bis(1-adamantyl)-butyl-phosphane; methanesulfonate (13.08 mg, 17.96 μmol, 0.1 equiv.), and KF (31.30 mg, 538.74 μmol, 12.62 μL, 3 equiv.) in dioxane (4 mL) and H₂O (1 mL) was degassed and purged with N₂ three times and then the mixture was stirred at 100° C. for 1 hour under N₂ atmosphere. The reaction mixture was filtered and concentrated under reduced pressure to give a residue. The residue was purified by column chromatography (SiO₂, dichloromethane: methanol=100/1 to 30/1) to give tert-butyl 6-[8-(1,3-benzothiazol-2-ylcarbamoyl)-3,4-dihydro-1H-isoquinolin-2-yl]-3-[3-[4-[(3S)-3-[4-[3-(2,6-dioxo-3-piperidyl)-1-methyl-indazol-7-yl]piperazin-1-yl]butyl]cyclohexoxy]-2-methyl-phenyl]pyridine-2-carboxylate (100 mg, 94.6 μmol, 52.7% yield) as a yellow solid.

MS (ESI) m/z: 1056.5 [M+H]⁺.

### Step C. Procedure for Preparation of 6-(8-(benzo[d]thiazol-2-ylcarbamoyl)-3,4-dihydroisoquinolin-2(1H)-yl)-3-(3-(((1R,4r)-4-((3S)-3-(4-(3-(2,6-dioxopiperidin-3-yl)-1-methyl-1H-indazol-7-yl)piperazin-1-yl)butyl)cyclohexyl)oxy)-2-methylphenyl)picolinic acid [6-(8-(benzo[d] thiazol-2-ylcarbamoyl)-3,4-dihydroisoquinolin-2(1H)-yl)-3-(3-(((1R,4r)-4-((3S*)-3-(4-(3-(2,6-dioxopiperidin-3-yl)-1-methyl-1H-indazol-7-yl)piperazin-1-yl)butyl)cyclohexyl)oxy)-2-methylphenyl)picolinic acid]

To a solution of tert-butyl 6-[8-(1,3-benzothiazol-2-ylcarbamoyl)-3,4-dihydro-1H-isoquinolin-2-yl]-3-[3-[4-[(3S)-3-[4-[3-(2,6-dioxo-3-piperidyl)-1-methyl-indazol-7-yl]piperazin-1-yl]butyl]cyclohexoxy]-2-methyl-phenyl]pyridine-2-carboxylate (100 mg, 94.67 μmol, 1 equiv.) in DCM (1 mL) was added TFA (1.54 g, 13.51 mmol, 1 mL, 142.67 equiv.) and stirred at 40° C. for 1 hour. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1R,4r)-4-((3S)-3-(4-(3-(2,6-dioxopiperidin-3-yl)-1-methyl-1H-indazol-7-yl)piperazin-1-yl)butyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1R,4r)-4-((3S*)-3-(4-(3-(2,6-dioxopiperidin-3-yl)-1-methyl-1H-indazol-7-yl)piperazin-1-yl)butyl)cyclohexyl)oxy)-2-methylphenyl)picolinic acid] (58.3 mg, 55.0 μmol, 58.1% yield, 98.7% purity) as a off-white solid.

MS (ESI) m/z: 1000.4 [M+H]⁺.

¹H NMR (400 MHz, DMSO-d₆) δ=13.05-12.61 (m, 1H), 10.89 (s, 1H), 8.14 (s, 1H), 8.04 (d, J=7.6 Hz, 1H), 7.79 (d, J=8.0 Hz, 1H), 7.63 (d, J=7.2 Hz, 1H), 7.50-7.43 (m, 3H), 7.42-7.33 (m, 3H), 7.11-7.00 (m, 3H), 6.98-6.90 (m, 2H), 6.62 (d, J=7.6 Hz, 1H), 4.98 (s, 2H), 4.37-4.32 (m, 1H), 4.25 (s, 3H), 4.21 (s, 1H), 3.92 (t, J=5.2 Hz, 2H), 3.27-3.23 (m, 4H), 3.03 (t, J=5.2 Hz, 2H), 2.95-2.82 (m, 4H), 2.66-2.54 (m, 3H), 2.34-2.27 (m, 1H), 2.20-2.14 (m, 1H), 2.09 (d, J=9.6 Hz, 2H), 1.88 (s, 3H), 1.85-1.79 (m, 2H), 1.68-1.58 (m, 1H), 1.36 (d, J=2.4 Hz, 2H), 1.33-1.22 (m, 4H), 1.17-1.08 (m, 2H), 1.07 (s, 3H)

### Example 210. Preparation of Compound 284a

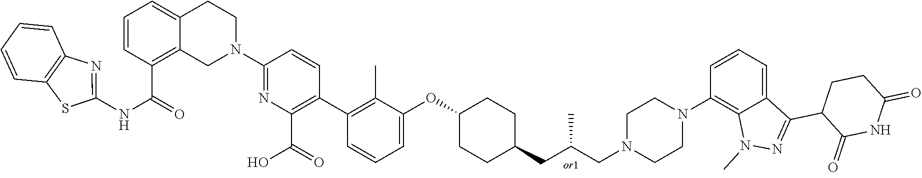 [SMILES: Cc1c(O[C@H]2CC[C@H](C[C@H](C)CN3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1S,4r)-4-((2S)-3-(4-(3-(2,6-dioxopiperidin-3-yl)-1-methyl-1H-indazol-7-yl)piperazin-1-yl)-2-methylpropyl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1S,4r)-4-((2S*)-3-(4-(3-(2,6-dioxopiperidin-3-yl)-1-methyl-1H-indazol-7-yl)piperazin-1-yl)-2-methylpropyl)cyclohexyl)oxy)-2-methylphenyl)picolinic acid]

### Step A. Procedure for Preparation of (S)-3-((1r,4S)-4-(3-bromo-2-methylphenoxy)cyclohexyl)-2-methylpropanal

To a solution of oxalyl dichloride (74.38 mg, 586.03 μmol, 51.30 μL, 2 equiv.) in DCM (5 mL) was added dropwise to a solution of DMSO (91.58 mg, 1.17 mmol, 91.58 μL, 4 equiv.) in DCM (2 mL) at −70° C. under N₂ atmosphere. The mixture was stirred at −70° C. for 1 hour. After which time (2S)-3-[4-(3-bromo-2-methyl-phenoxy)cyclohexyl]-2-methyl-propan-1-ol (100 mg, 293.01 μmol, 1 equiv.) in DCM (2 mL) was added dropwise at −70° C. The solution was stirred for 1 hour at −70° C. Then TEA (177.90 mg, 1.76 mmol, 244.70 μL, 6 equiv.) was added into the solution. The solution was stirred at −70° C. for 0.5 hour under N₂ atmosphere. Water (30 mL) was then added and extracted with EtOAc 20 mL (10 mL×2), dried over anhydrous sodium sulfate, filtered and concentrated under vacuum to give (S)-3-((1r,4S)-4-(3-bromo-2-methylphenoxy)cyclohexyl)-2-methylpropanal (80 mg, crude) as a yellow oil.

### Step B. Procedure for Preparation of 3-(7-(4-((S)-3-((1r,4S)-4-(3-bromo-2-methylphenoxy)cyclohexyl)-2-methylpropyl)piperazin-1-yl)-1-methyl-1H-indazol-3-yl)piperidine-2,6-dione

To a solution of 3-(1-methyl-7-(piperazin-1-yl)-1H-indazol-3-yl)piperidine-2,6-dione (102.95 mg, 282.96 μmol, 1.2 equiv., HCl) in DCM (1 mL) was added NaBH(OAc)₃ (149.93 mg, 707.41 μmol, 3 equiv.), then (S)-3-((1r,4S)-4-(3-bromo-2-methylphenoxy)cyclohexyl)-2-methylpropanal (80 mg, 235.80 μmol, 1 equiv.) was added into the mixture and stirred at 0° C. for 1 hour. Then the mixture was stirred at 25° C. for 15 hours. The mixture was filtered and concentrated under vacuum to give a residue. The residue was purified by column chromatography (SiO₂, petroleum ether/ethyl acetate=4/1 to DCM:MeOH=10:1) to give 3-(7-(4-((S)-3-((1r,4S)-4-(3-bromo-2-methylphenoxy)cyclohexyl)-2-methylpropyl)piperazin-1-yl)-1-methyl-1H-indazol-3-yl)piperidine-2,6-dione (40 mg, 61.4 μmol, 26.0% yield) as a yellow oil.

MS (ESI) m/z: 650.2 [M+H]⁺.

### Step C. Procedure for Preparation of tert-butyl 6-(8-(benzo[d]thiazol-2-ylcarbamoyl)-3,4-dihydroisoquinolin-2(1H)-yl)-3-(3-(((1S,4r)-4-((2S)-3-(4-(3-(2,6-dioxopiperidin-3-yl)-1-methyl-1H-indazol-7-yl)piperazin-1-yl)-2-methylpropyl)cyclohexyl)oxy)-2-methylphenyl)picolinate

A mixture of 3-(7-(4-((S)-3-((1r,4S)-4-(3-bromo-2-methylphenoxy)cyclohexyl)-2-methylpropyl)piperazin-1-yl)-1-methyl-1H-indazol-3-yl)piperidine-2,6-dione (40 mg, 61.48 μmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (45.19 mg, 73.77 μmol, 1.2 equiv.), KF (10.71 mg, 184.43 μmol, 4.32 μL, 3 equiv.), H₂O (0.1 mL), and [2-(2-aminophenyl)phenyl]palladium(1+); bis(1-adamantyl)-butyl-phosphane; methanesulfonate (4.48 mg, 6.15 μmol, 0.1 equiv.) in dioxane (1 mL) was degassed and purged with N₂ three times, and then the mixture was stirred at 100° C. for 1 hour under N₂ atmosphere. The reaction mixture was concentrated under reduced pressure to give a residue. The residue was purified by column chromatography (SiO₂, petroleum ether/ethyl acetate=3/1, DCM:MeOH=10:1) to give tert-butyl 6-(8-(benzo[d]thiazol-2-ylcarbamoyl)-3,4-dihydroisoquinolin-2(1H)-yl)-3-(3-(((1S,4r)-4-((2S)-3-(4-(3-(2,6-dioxopiperidin-3-yl)-1-methyl-1H-indazol-7-yl)piperazin-1-yl)-2-methylpropyl)cyclohexyl)oxy)-2-methylphenyl)picolinate (30 mg, 28.4 μmol, 46.2% yield) as a yellow solid.

MS (ESI) m/z: 1056.4 [M+H]⁺.

### Step D. Procedure for Preparation of 6-(8-(benzo[d]thiazol-2-ylcarbamoyl)-3,4-dihydroisoquinolin-2(1H)-yl)-3-(3-(((1S,4r)-4-((2S)-3-(4-(3-(2,6-dioxopiperidin-3-yl)-1-methyl-1H-indazol-7-yl)piperazin-1-yl)-2-methylpropyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1S,4r)-4-((2S*)-3-(4-(3-(2,6-dioxopiperidin-3-yl)-1-methyl-1H-indazol-7-yl)piperazin-1-yl)-2-methylpropyl)cyclohexyl)oxy)-2-methylphenyl)picolinic acid]

To a solution of tert-butyl 6-(8-(benzo[d]thiazol-2-ylcarbamoyl)-3,4-dihydroisoquinolin-2(1H)-yl)-3-(3-(((1S,4r)-4-((2S)-3-(4-(3-(2,6-dioxopiperidin-3-yl)-1-methyl-1H-indazol-7-yl)piperazin-1-yl)-2-methylpropyl)cyclohexyl)oxy)-2-methylphenyl)picolinate (30 mg, 28.40 μmol, 1 equiv.) in DCM (1 mL) was added TFA (770.00 mg, 6.75 mmol, 0.5 mL, 237.78 equiv.). The mixture was stirred at 25° C. for 8 hours. The reaction mixture was concentrated under reduced pressure to remove solvent and to give a residue. The residue was purified by prep-HPLC to give 6-(8-(benzo[d]thiazol-2-ylcarbamoyl)-3,4-dihydroisoquinolin-2(1H)-yl)-3-(3-(((1S,4r)-4-((2S)-3-(4-(3-(2,6-dioxopiperidin-3-yl)-1-methyl-1H-indazol-7-yl)piperazin-1-yl)-2-methylpropyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1S,4r)-4-((2S*)-3-(4-(3-(2,6-dioxopiperidin-3-yl)-1-methyl-1H-indazol-7-yl)piperazin-1-yl)-2-methylpropyl)cyclohexyl)oxy)-2-methylphenyl)picolinic acid] (15.4 mg, 14.4 μmol, 50.7% yield, 97.6% purity) as a yellow solid.

MS (ESI) m/z: 1000.5 [M+H]⁺

¹H NMR (400 MHz, DMSO) δ=10.87 (s, 1H), 8.22 (s, 1H), 8.01 (d, J=8.0 Hz, 1H), 7.80-7.74 (m, 1H), 7.63 (d, J=6.8 Hz, 1H), 7.48-7.32 (m, 6H), 7.07-6.98 (m, 3H), 6.94-6.85 (m, 2H), 6.64 (d, J=7.6 Hz, 1H), 5.75 (s, 1H), 5.01-4.94 (m, 2H), 4.36-4.31 (m, 1H), 4.24 (s, 3H), 4.22-4.15 (m, 1H), 3.93-3.88 (m, 2H), 3.02 (t, J=5.6 Hz, 2H), 2.64-2.54 (m, 8H), 2.21-2.04 (m, 6H), 1.88 (s, 3H), 1.84-1.72 (m, 3H), 1.49-1.24 (m, 5H), 1.14-1.07 (m, 1H), 1.03-0.96 (m, 2H), 0.91-0.85 (m, 3H)

### Example 211. Preparation of Compound 283b

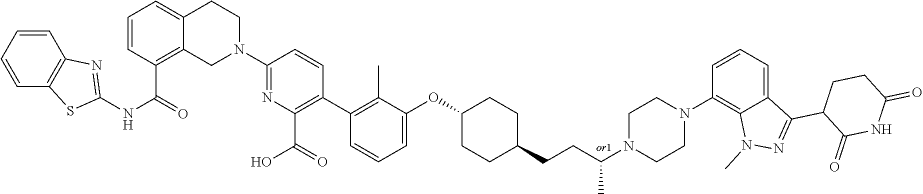 [SMILES: Cc1c(O[C@H]2CC[C@H](CC[C@@H](C)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1S,4r)-4-((3R)-3-(4-(3-(2,6-dioxopiperidin-3-yl)-1-methyl-1H-indazol-7-yl)piperazin-1-yl)butyl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1S,4r)-4-((3R*)-3-(4-(3-(2,6-dioxopiperidin-3-yl)-1-methyl-1H-indazol-7-yl)piperazin-1-yl)butyl)cyclohexyl)oxy)-2-methylphenyl)picolinic acid]

### Step A. Procedure for Preparation of 3-[7-[4-[(1R)-3-[4-(3-bromo-2-methyl-phenoxy) cyclohexyl]-1-methyl-propyl]piperazin-1-yl]-1-methyl-indazol-3-yl]piperidine-2, 6-dione

To a solution of [(1S)-3-[4-(3-bromo-2-methyl-phenoxy)cyclohexyl]-1-methyl-propyl]4-methylbenzenesulfonate (80 mg, 161.46 μmol, 1 equiv.) in CH₃CN (1 mL) was added 3-(1-methyl-7-piperazin-1-yl-indazol-3-yl)piperidine-2,6-dione (63.43 mg, 193.75 μmol, 1.2 equiv.), K₂CO₃ (66.95 mg, 484.38 μmol, 3 equiv.), and KI (26.80 mg, 161.46 μmol, 1 equiv.) at 25° C. The reaction was stirred at 60° C. for 16 hours. The reaction mixture was filtered and concentrated under reduced pressure to give a residue. The residue was purified by flash silica gel chromatography (eluent of 0˜10% ethyl acetate/petroleum ether). The compound 3-[7-[4-[(1R)-3-[4-(3-bromo-2-methyl-phenoxy)cyclohexyl]-1-methyl-propyl]piperazin-1-yl]-1-methyl-indazol-3-yl]piperidine-2,6-dione (68 mg, 101.1 μmol, 62.6% yield, 96.8% purity) was obtained as a yellow oil.

MS (ESI) m/z: 650.2 [M+H]⁺.

### Step B. Procedure for Preparation of tert-butyl 6-[8-(1, 3-benzothiazol-2-ylcarbamoyl)-3, 4-dihydro-1H-isoquinolin-2-yl]-3-[3-[4-[(3R)-3-[4-[3-(2,6-dioxo-3-piperidyl)-1-methyl-indazol-7-yl]piperazin-1-yl]butyl]cyclohexoxy]-2-methyl-phenyl]pyridine-2-carboxylate

tert-Butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (70.42 mg, 114.96 μmol, 1.1 equiv.), 3-[7-[4-[(1R)-3-[4-(3-bromo-2-methyl-phenoxy)cyclohexyl]-1-methyl-propyl]piperazin-1-yl]-1-methyl-indazol-3-yl]piperidine-2,6-dione (68 mg, 104.51 μmol, 1 equiv.), KF (18.22 mg, 313.53 mol, 7.35 μL, 3 equiv.), and [2-(2-aminophenyl)phenyl]palladium (1+); bis(1-adamantyl)-butyl-phosphane; methanesulfonate (15.22 mg, 20.90 μmol, 0.2 equiv.) were taken up into a microwave tube in dioxane (2 mL) and H₂O (0.2 mL). The sealed tube was heated at 100° C. for 60 min under microwave. The reaction mixture was filtered and concentrated under reduced pressure to give a residue. The residue was purified by flash silica gel chromatography (eluent of 0˜30% ethyl acetate/petroleum ether). The compound tert-butyl 6-[8-(1,3-benzothiazol-2-ylcarbamoyl)-3,4-dihydro-1H-isoquinolin-2-yl]-3-[3-[4-[(3R)-3-[4-[3-(2,6-dioxo-3-piperidyl)-1-methyl-indazol-7-yl]piperazin-1-yl]butyl]cyclohexoxy]-2-methyl-phenyl]pyridine-2-carboxylate (100 mg, 53.9 mol, 51.6% yield, 57% purity) was obtained as a yellow solid.

MS (ESI) m/z: 1056.4 [M+H]⁺.

### Step C. Procedure for Preparation of 6-(8-(benzo[d]thiazol-2-ylcarbamoyl)-3,4-dihydroisoquinolin-2(1H)-yl)-3-(3-(((1S,4r)-4-((3R)-3-(4-(3-(2,6-dioxopiperidin-3-yl)-1-methyl-1H-indazol-7-yl)piperazin-1-yl)butyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1S,4r)-4-((3R*)-3-(4-(3-(2,6-dioxopiperidin-3-yl)-1-methyl-1H-indazol-7-yl)piperazin-1-yl)butyl)cyclohexyl)oxy)-2-methylphenyl)picolinic acid]

A solution of tert-butyl 6-[8-(1,3-benzothiazol-2-ylcarbamoyl)-3,4-dihydro-1H-isoquinolin-2-yl]-3-[3-[4-[(3R)-3-[4-[3-(2,6-dioxo-3-piperidyl)-1-methyl-indazol-7-yl]piperazin-1-yl]butyl]cyclohexoxy]-2-methyl-phenyl]pyridine-2-carboxylate (100 mg, 53.96 μmol, 57% purity, 1 equiv.) in DCM (0.5 mL) and TFA (0.5 mL) was stirred at 25° C. for 16 hours. The reaction mixture was filtered and concentrated under reduced pressure to give a residue. The residue was purified by prep-HPLC to give compound 6-(8-(benzo[d]thiazol-2-ylcarbamoyl)-3,4-dihydroisoquinolin-2(1H)-yl)-3-(3-(((1S,4r)-4-((3R)-3-(4-(3-(2,6-dioxopiperidin-3-yl)-1-methyl-1H-indazol-7-yl)piperazin-1-yl)butyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1S,4r)-4-((3R*)-3-(4-(3-(2,6-dioxopiperidin-3-yl)-1-methyl-1H-indazol-7-yl)piperazin-1-yl)butyl)cyclohexyl)oxy)-2-methylphenyl)picolinic acid] (17.5 mg, 15.9 μmol, 29.5% yield, 90.7% purity) was obtained as an off-white solid.

MS (ESI) m/z: 1000.4 [M+H]⁺.

¹H NMR (400 MHz, DMSO-d₆) δ=10.89 (s, 1H), 8.03 (d, J=8.0 Hz, 1H), 7.79 (d, J=8.0 Hz, 1H), 7.62 (d, J=7.2 Hz, 1H), 7.50-7.43 (m, 4H), 7.40-7.32 (m, 2H), 7.10-7.02 (m, 3H), 6.94 (dd, J=8.4, 15.6 Hz, 2H), 6.62 (d, J=7.6 Hz, 1H), 4.98 (s, 2H), 4.35 (dd, J=5.2, 9.2 Hz, 1H), 4.30-4.19 (m, 4H), 3.91 (t, J=5.2 Hz, 2H), 3.02 (t, J=5.2 Hz, 3H), 2.75-2.52 (m, 5H), 2.44-2.23 (m, 2H), 2.23-2.04 (m, 4H), 1.87 (s, 3H), 1.83 (s, 2H), 1.65-0.86 (m, 15H)

### Example 212. Preparation of Compound 278a

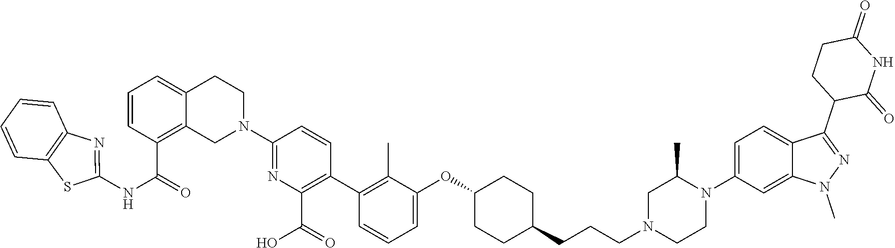 [SMILES: Cc1c(O[C@H]2CC[C@H](CCCN3CCN(c4ccc5c(C6CCC(=O)NC6=O)nn(C)c5c4)[C@H](C)C3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1S,4r)-4-(3-((3R)-4-(3-(2,6-dioxopiperidin-3-yl)-1-methyl-1H-indazol-6-yl)-3-methylpiperazin-1-yl)propyl)cyclohexyl)oxy)-2-methylphenyl)picolinic acid

### Step A. Procedure for Preparation of methyl 4-(3-bromo-2-methyl-phenoxy)cyclohexanecarboxylate

To a solution of methyl 4-hydroxycyclohexanecarboxylate (16 g, 101.14 mmol, 1.0 equiv.) and 3-bromo-2-methyl-phenol (20.81 g, 111.26 mmol, 1.1 equiv.) in toluene (500 mL) was added 2-(tributyl-λ5-phosphanylidene)acetonitrile (29.29 g, 121.37 mmol, 1.2 equiv.). The mixture was stirred at 120° C. for 12 hours. The reaction mixture was then concentrated under reduced pressure to give the compound methyl 4-(3-bromo-2-methyl-phenoxy)cyclohexanecarboxylate (125 g, crude) was obtained as a black oil, which was used in the next step without further purification.

### Step B. Procedure for Preparation of [4-(3-bromo-2-methyl-phenoxy)cyclohexyl]methanol

A mixture of methyl 4-(3-bromo-2-methyl-phenoxy)cyclohexanecarboxylate (28 g, 34.23 mmol, 40% purity, 1.0 equiv.) in THE (300 mL) was slowly added LAH (1.36 g, 35.94 mmol, 1.05 equiv.) at 0° C. The mixture was then stirred at 0° C. for 1 hour. The mixture was quenched by 2.5 g Na₂SO₄·10 H₂O. The solution was poured into ice-water (250 mL) slowly and the pH was adjusted to pH 4-5 with 1 M HCl, extracted with EtOAc 600 mL (200 mL×3), washed with brine 300 mL (150 mL×2), dried over by sodium sulfate, filtered, and concentrated under reduced pressure. The residue was purified by flash silica gel chromatography (eluent of 0-18% ethyl acetate/petroleum ether). The compound [4-(3-bromo-2-methyl-phenoxy)cyclohexyl]methanol (10 g, 32.3 mmol, 47.2% yield, 96.7% purity) was obtained as a brown oil.

### Step C. Procedure for Preparation of 4-(3-bromo-2-methyl-phenoxy)cyclohexanecarbaldehyde

To a solution of DMSO (5.22 g, 66.84 mmol, 5.22 mL, 4 equiv.) in DCM (200 mL) was added the mixture of (COCl)₂ (4.24 g, 33.42 mmol, 2.93 mL, 2 equiv.) and DCM at −78° C. and stirred for 1 hour. Then [4-(3-bromo-2-methyl-phenoxy)cyclohexyl]methanol (5 g, 16.71 mmol, 1.0 equiv.) was added into the mixture and stirred for 1 hour. TEA (10.15 g, 100.27 mmol, 13.96 mL, 6.0 equiv.) was then added into the mixture and stirred at −70° C. for 1 hour. The reaction mixture was diluted with H₂O (200 mL) and extracted with DCM 60 mL (30 mL×2). The combined organic layers were dried over sodium sulfate and concentrated under reduced pressure to give 4-(3-bromo-2-methyl-phenoxy)cyclohexanecarbaldehyde (17 g, crude) as a yellow oil, which was used in the next step without further purification.

### Step D. Procedure for Preparation of ethyl (E)-3-[4-(3-bromo-2-methyl-phenoxy)cyclohexyl]prop-2-enoate

A mixture of 4-(3-bromo-2-methyl-phenoxy)cyclohexanecarbaldehyde (17 g, 57.20 mmol, 1.0 equiv.), ethyl 2-diethoxyphosphorylacetate (17.95 g, 80.08 mmol, 15.89 mL, 1.4 equiv.), and NaH (3.66 g, 91.52 mmol, 60% purity, 1.6 equiv.) in THE (200 mL) was degassed and purged with N₂ three times, and then the mixture was stirred at 0° C. for 2 hours under N₂ atmosphere. The reaction mixture was quenched by addition NH₄Cl (50 mL) at 25° C., and then diluted with NH₄Cl (50 mL) and extracted with ethyl acetate 300 mL (100 mL×3). The combined organic layers were then dried over sodium sulfate, filtered and concentrated under reduced pressure to give a residue. The residue was purified by flash silica gel chromatography (eluent of 0-5% ethyl acetate/petroleum ether). The compound ethyl (E)-3-[4-(3-bromo-2-methyl-phenoxy)cyclohexyl]prop-2-enoate (8 g, 21.78 mmol, 38.0% yield) was obtained as a white solid.

¹H NMR (400 MHz, DMSO-d₆) δ=7.17-7.13 (m, 1H), 7.11-7.05 (m, 1H), 7.05-7.00 (m, 1H), 6.85 (dd, J=6.8, 15.8 Hz, 1H), 5.83 (dd, J=1.2, 15.8 Hz, 1H), 4.31-4.20 (m, 1H), 4.11 (q, J=7.2 Hz, 2H), 2.23 (m, 1H), 2.21 (s, 3H), 2.12-2.04 (m, 2H), 1.82 (d, J=11.2 Hz, 2H), 1.49-1.39 (m, 2H), 1.37-1.26 (m, 2H), 1.21 (t, J=7.2 Hz, 3H)

### Step E. Procedure for Preparation of ethyl 3-[4-(3-bromo-2-methyl-phenoxy)cyclohexyl]propanoate

A mixture of ethyl (E)-3-[4-(3-bromo-2-methyl-phenoxy)cyclohexyl]prop-2-enoate (8 g, 21.78 mmol, 1 equiv.) and PtO₂ (4.95 g, 21.78 mmol, 1.0 equiv.) in EtOH (100 mL) was degassed and purged with H₂ three times, and then the mixture was stirred at 25° C. for 2 hours under H₂ atmosphere. The reaction mixture was filtered and concentrated under reduced pressure to give a residue. The compound ethyl 3-[4-(3-bromo-2-methyl-phenoxy)cyclohexyl]propanoate (8 g, crude) was obtained as a brown oil.

### Step F. Procedure for Preparation of 3-[4-(3-bromo-2-methyl-phenoxy)cyclohexyl]propan-1-01

To a mixture of ethyl 3-[4-(3-bromo-2-methyl-phenoxy)cyclohexyl]propanoate (8 g, 21.66 mmol, 1.0 equiv.) in THE (100 mL) was slowly added LAH (986.65 mg, 26.00 mmol, 1.2 equiv.) at 0° C. and then the mixture was stirred at 0° C. for 1.5 hours. The mixture was quenched by 2.0 g Na₂SO₄·10 H₂O. The solution was slowly poured into ice-water (80 mL) and the pH was acidized to pH 4˜5 with 1 M HCl. The mixture was extracted with EtOAc 180 mL (60 mL×3), washed with brine 100 mL (50 mL×2), dried by sodium sulfate, filtered, and concentrated under reduced pressure to give compound 3-[4-(3-bromo-2-methyl-phenoxy)cyclohexyl]propan-1-ol (5 g, crude) as a colorless oil.

¹H NMR (400 MHz, DMSO-d₆) δ=7.15-7.11 (m, 1H), 7.06 (t, J=8.0 Hz, 1H), 7.02-6.99 (m, 1H), 4.36 (t, J=5.2 Hz, 1H), 4.21 (m, J=4.4, 10.6 Hz, 1H), 3.39-3.37 (m, 2H), 2.20 (s, 3H), 2.07-2.01 (m, 2H), 1.78-1.72 (m, 2H), 1.41 (dd, J=7.2, 14.7 Hz, 3H), 1.37-1.29 (m, 2H), 1.22-1.18 (m, 2H), 1.07-0.97 (m, 2H)

### Step G. Procedure for Preparation of 3-[4-(3-bromo-2-methyl-phenoxy)cyclohexyl]propanal

To a solution of DMSO (2.39 g, 30.56 mmol, 2.39 mL, 4.0 equiv.) in DCM (40 mL) was added (COCl)₂ (1.94 g, 15.28 mmol, 1.34 mL, 2.0 equiv.) under −78° C. and stirred for 1 hour. Then 3-[4-(3-bromo-2-methyl-phenoxy)cyclohexyl]propan-1-ol (2.5 g, 7.64 mmol, 1.0 equiv.) was added into the mixture and stirred for 1 hour. TEA (4.64 g, 45.84 mmol, 6.38 mL, 6.0 equiv.) was then added into the mixture and stirred at −70° C. for 1 hour. The reaction mixture was diluted with H₂O (30 mL), extracted with DCM 30 mL (15 mL×2), dried over sodium sulfate, and concentrated under reduced pressure to give the compound 3-[4-(3-bromo-2-methyl-phenoxy)cyclohexyl]propanal (2.5 g, crude) as a yellow oil, which was used in the next step without further purification.

¹H NMR (400 MHz, DMSO-d₆) δ=9.67 (t, J=1.6 Hz, 1H), 7.16-7.11 (m, 1H), 7.06 (t, J=8.0 Hz, 1H), 7.03-6.99 (m, 1H), 4.27-4.17 (m, 1H), 2.45 (m, J=1.6, 7.6 Hz, 2H), 2.20 (s, 3H), 2.08-2.00 (m, 2H), 1.80-1.71 (m, 2H), 1.45 (q, J=7.2 Hz, 2H), 1.40-1.30 (m, 2H), 1.30-1.23 (m, 1H), 1.10-0.97 (m, 2H)

### Step H. Procedure for Preparation of tert-butyl 6-[8-(1,3-benzothiazol-2-ylcarbamoyl)-3,4-dihydro-1H-isoquinolin-2-yl]-3-[2-methyl-3-[4-(3-oxopropyl)cyclohexoxy]phenyl]pyridine-2-carboxylate

A mixture of 3-[4-(3-bromo-2-methyl-phenoxy)cyclohexyl]propanal (500 mg, 1.54 mmol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (847.51 mg, 1.38 mmol, 0.9 equiv.), K₂CO₃ (318.70 mg, 2.31 mmol, 1.5 equiv.), Ad₂nBuP Pd G₃ (cataCXium® A Pd G₃) (75 mg, 102.98 μmol, 0.2 equiv.) in dioxane (5 mL) was degassed and purged with N₂ three times, and then the mixture was stirred at 100° C. for 1 hour under microwave. The reaction mixture was filtered and concentrated under reduced pressure to remove solvent. The residue was purified by flash silica gel chromatography (eluent of 0˜35% ethyl acetate/petroleum ether). The compound tert-butyl 6-[8-(1,3-benzothiazol-2-ylcarbamoyl)-3,4-dihydro-1H-isoquinolin-2-yl]-3-[2-methyl-3-[4-(3-oxopropyl)cyclohexoxy]phenyl]pyridine-2-carboxylate (700 mg, 957.7 μmol, 31.1% yield) was obtained as a yellow solid.

MS (ESI) m/z: 731.4 [M+H]⁺

### Step I. Procedure for preparation of tert-butyl (3R)-4-[3-(2,6-dibenzyloxy-3-pyridyl)-1-methyl-indazol-6-yl]-3-methyl-piperazine-1-carboxylate

A mixture of 6-bromo-3-(2,6-dibenzyloxy-3-pyridyl)-1-methyl-indazole (1.5 g, 3.00 mmol, 1.0 equiv.), tert-butyl (3R)-3-methylpiperazine-1-carboxylate (1.20 g, 6.00 mmol, 2.0 equiv.), 1,3-bis[2,6-bis(1-propylbutyl)phenyl]-4,5-dichloro-2H-imidazol-1-ium-2-ide; 3-chloropyridine; dichloropalladium (291.61 mg, 299.77 μmol, 0.1 equiv.), and Cs₂CO₃ (2.93 g, 8.99 mmol, 3 equiv.) in 2-methyl-2-butanol (20 mL) was degassed and purged with N₂ three times, and then the mixture was stirred at 100° C. for 2 hours under N₂ atmosphere. The reaction mixture was filtered and concentrated under reduced pressure to remove solvent. The residue was purified by flash silica gel chromatography (eluent of 0˜35% ethyl acetate/petroleum ether). The compound tert-butyl (3R)-4-[3-(2,6-dibenzyloxy-3-pyridyl)-1-methyl-indazol-6-yl]-3-methyl-piperazine-1-carboxylate (580 mg, 935.8 μmol, 31.2% yield) was obtained as a yellow oil.

MS (ESI) m/z: 620.4 [M+H]⁺

### Step J. Procedure for Preparation of tert-butyl (3R)-4-[3-(2,6-dioxo-3-piperidyl)-1-methyl-indazol-6-yl]-3-methyl-piperazine-1-carboxylate

A mixture of tert-butyl (3R)-4-[3-(2,6-dibenzyloxy-3-pyridyl)-1-methyl-indazol-6-yl]-3-methyl-piperazine-1-carboxylate (580 mg, 935.86 μmol, 1.0 equiv.), Pd/C (200 mg, 10% purity, 1.0 equiv.), Pd(OH)₂ (200 mg, 1.42 mmol, 1.52 equiv.), and AcOH (168.60 mg, 2.81 mmol, 160.57 μL, 3.0 equiv.) in EtOH (5 mL) and THE (5 mL) was degassed and purged with H₂ three times, and then the mixture was stirred at 25° C. for 12 hours under H₂ atmosphere. The mixture was filtered, washed with THE (30 mL), and concentrated under reduced pressure to give the compound tert-butyl (3R)-4-[3-(2,6-dioxo-3-piperidyl)-1-methyl-indazol-6-yl]-3-methyl-piperazine-1-carboxylate (480 mg, crude) as a yellow solid.

MS (ESI) m/z: 442.3 [M+H]⁺

### Step K. Procedure for Preparation of 3-[1-methyl-6-[(3S)-3-methyl-4-piperidyl]indazol-3-yl]piperidine-2,6-dione

A mixture of tert-butyl (3R)-4-[3-(2,6-dioxo-3-piperidyl)-1-methyl-indazol-6-yl]-3-methyl-piperazine-1-carboxylate (480 mg, 858.85 μmol, 79% purity, 1.0 equiv.) in HCl/EtOAc (7 mL) was degassed and purged with N₂ three times, and then the mixture was stirred at 25° C. for 2 hours under N₂ atmosphere. The reaction mixture was concentrated under reduced pressure to remove solvent. The compound 3-[1-methyl-6-[(3S)-3-methyl-4-piperidyl]indazol-3-yl]piperidine-2,6-dione (470 mg, crude) was obtained as a brown solid.

MS (ESI) m/z: 342.2 [M+H]⁺

### Step L. Procedure for Preparation of tert-butyl 6-[8-(1,3-benzothiazol-2-ylcarbamoyl)-3,4-dihydro-1H-isoquinolin-2-yl]-3-[3-[4-[3-[(3R)-4-[3-(2,6-dioxo-3-piperidyl)-1-methyl-indazol-6-yl]-3-methyl-piperazin-1-yl]propyl]cyclohexoxy]-2-methyl-phenyl]pyridine-2-carboxylate

To a solution of 3-[1-methyl-6-[(3S)-3-methyl-4-piperidyl]indazol-3-yl]piperidine-2,6-dione (100 mg, 293.76 μmol, 1 equiv.) in DCM (3 mL) was added the mixture of tert-butyl 6-[8-(1,3-benzothiazol-2-ylcarbamoyl)-3,4-dihydro-1H-isoquinolin-2-yl]-3-[2-methyl-3-[4-(3-oxopropyl)cyclohexoxy]phenyl]pyridine-2-carboxylate (150.30 mg, 205.63 μmol, 0.7 equiv.) and acetic acid at 25° C. The mixture was stirred for 11 hours. NaBH(OAc)₃ (186.78 mg, 881.27 μmol, 3.0 equiv.) was added into the mixture and stirred for 1 hour. The reaction mixture was concentrated under reduced pressure to remove solvent. The residue was purified by flash silica gel chromatography (eluent of 0-87% ethyl acetate/petroleum ether). The compound tert-butyl 6-[8-(1,3-benzothiazol-2-ylcarbamoyl)-3,4-dihydro-1H-isoquinolin-2-yl]-3-[3-[4-[3-[(3R)-4-[3-(2,6-dioxo-3-piperidyl)-1-methyl-indazol-6-yl]-3-methyl-piperazin-1-yl]propyl]cyclohexoxy]-2-methyl-phenyl]pyridine-2-carboxylate (140 mg, 132.5 μmol, 45.1% yield) was obtained as a yellow solid.

MS (ESI) m/z: 529.1 [M12+H]⁺

### Step M. Procedure for Preparation of 6-(8-(benzo[d]thiazol-2-ylcarbamoyl)-3,4-dihydroisoquinolin-2(1H)-yl)-3-(3-(((1S,4r)-4-(3-((3R)-4-(3-(2,6-dioxopiperidin-3-yl)-1-methyl-1H-indazol-6-yl)-3-methylpiperazin-1-yl)propyl)cyclohexyl)oxy)-2-methylphenyl)picolinic acid

A solution of tert-butyl 6-[8-(1,3-benzothiazol-2-ylcarbamoyl)-3,4-dihydro-1H-isoquinolin-2-yl]-3-[3-[4-[3-[(3R)-4-[3-(2,6-dioxo-3-piperidyl)-1-methyl-indazol-6-yl]-3-methyl-piperazin-1-yl]propyl]cyclohexoxy]-2-methyl-phenyl]pyridine-2-carboxylate (140 mg, 132.54 μmol, 1.0 equiv.) in DCM (1.5 mL) and TFA (1.5 mL) was stirred at 25° C. for 16 hours. The reaction mixture was concentrated under reduced pressure to remove solvent. The residue was purified by prep-HPLC to give compound 6-(8-(benzo[d]thiazol-2-ylcarbamoyl)-3,4-dihydroisoquinolin-2(1H)-yl)-3-(3-(((1S,4r)-4-(3-((3R)-4-(3-(2,6-dioxopiperidin-3-yl)-1-methyl-1H-indazol-6-yl)-3-methylpiperazin-1-yl)propyl)cyclohexyl)oxy)-2-methylphenyl)picolinic acid (20.2 mg, 19.0 μmol, 14.3% yield, 94.2% purity) as a brown solid.

MS (ESI) m/z: 501.1 [M12+H]⁺

¹H NMR (400 MHz, DMSO-d₆) δ=12.86 (s, 1H), 10.90-10.84 (m, 1H), 9.16 (s, 1H), 8.03 (d, J=7.6 Hz, 1H), 7.79 (d, J=8.0 Hz, 1H), 7.62 (d, J=7.2 Hz, 1H), 7.56 (d, J=8.8 Hz, 1H), 7.50-7.41 (m, 3H), 7.37 (m, 2H), 7.08 (t, J=7.8 Hz, 1H), 6.99-6.89 (m, 4H), 6.62 (d, J=8.0 Hz, 1H), 4.98 (s, 2H), 4.49 (d, J=2.0 Hz, 1H), 4.25 (s, 2H), 3.97 (s, 1H), 3.94-3.89 (m, 4H), 3.73-3.49 (m, 3H), 3.19-3.12 (m, 3H), 3.03 (t, J=5.2 Hz, 3H), 2.68-2.61 (m, 2H), 2.54 (s, 4H), 2.33 (s, 1H), 2.19-2.09 (m, 3H), 1.89-1.80 (m, 5H), 1.45-1.31 (m, 3H), 1.24 (d, J=4.6 Hz, 2H), 1.09 (d, J=7.0 Hz, 3H), 0.96 (d, J=6.8 Hz, 1H)

### Example 213. Preparation of Compound 273a

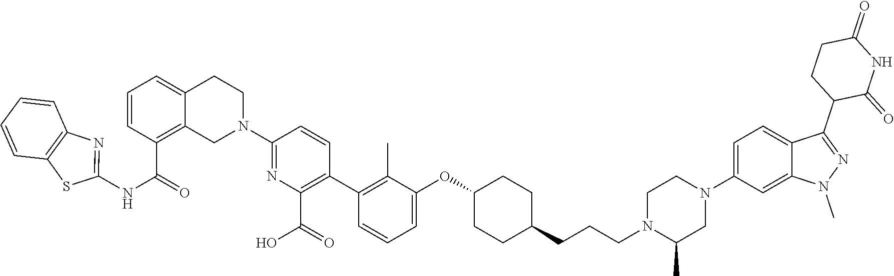 [SMILES: Cc1c(O[C@H]2CC[C@H](CCCN3CCN(c4ccc5c(C6CCC(=O)NC6=O)nn(C)c5c4)C[C@H]3C)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1S,4r)-4-(3-((2R)-4-(3-(2,6-dioxopiperidin-3-yl)-1-methyl-1H-indazol-6-yl)-2-methylpiperazin-1-yl)propyl)cyclohexyl)oxy)-2-methylphenyl)picolinic acid

### Step A. Procedure for Preparation of tert-butyl (2R)-4-[3-(2,6-dibenzyloxy-3-pyridyl)-1-methyl-indazol-6-yl]-2-methyl-piperazine-1-carboxylate

A mixture of 6-bromo-3-(2,6-dibenzyloxy-3-pyridyl)-1-methyl-indazole (1.5 g, 3.00 mmol, 1 equiv.), tert-butyl (2R)-2-methylpiperazine-1-carboxylate (1.80 g, 8.99 mmol, 3 equiv.), RuPhos (279.77 mg, 599.54 μmol, 0.2 equiv.), Pd₂(dba)₃ (274.50 mg, 299.77 μmol, 0.1 equiv.), and Cs₂CO₃ (2.93 g, 8.99 mmol, 3 equiv.) in toluene (2 mL) was degassed and purged with N₂ three times, and then the mixture was stirred at 110° C. for 16 hours under N₂ atmosphere. The reaction mixture was filtered and concentrated under reduced pressure to give a residue. The residue was purified by flash silica gel chromatography (eluent of 0-30% ethyl acetate/petroleum ether) to afford tert-butyl (2R)-4-[3-(2,6-dibenzyloxy-3-pyridyl)-1-methyl-indazol-6-yl]-2-methyl-piperazine-1-carboxylate (900 mg, 1.2 mmol, 42.6% yield, 88% purity) as a yellow oil.

MS (ESI) m/z: 620.5 [M+H]⁺.

### Step B. Procedure for Preparation of tert-butyl (2R)-4-[3-(2,6-dioxo-3-piperidyl)-1-methyl-indazol-6-yl]-2-methyl-piperazine-1-carboxylate

To a solution of tert-butyl (2R)-4-[3-(2,6-dibenzyloxy-3-pyridyl)-1-methyl-indazol-6-yl]-2-methyl-piperazine-1-carboxylate (1.05 g, 1.69 mmol, 1 equiv.) in EtOH (10 mL) and THE (10 mL) was added Pd/C (300 mg, 1.69 mmol, 10% purity, 1.00 equiv.), Pd(OH)₂ (300 mg, 213.62 μmol, 10% purity, 1.26e-1 equiv.), and AcOH (101.74 mg, 1.69 mmol, 96.90 μL, 1 equiv.) under N₂ atmosphere. The suspension was degassed and purged with H₂ three times. The mixture was then stirred under H₂ (15 Psi) at 25° C. for 16 hours. The reaction mixture was filtered and washed with THE (50 mL). The filtrate was concentrated to afford tert-butyl (2R)-4-[3-(2,6-dioxo-3-piperidyl)-1-methyl-indazol-6-yl]-2-methyl-piperazine-1-carboxylate (630 mg, crude) as a black brown oil, and was used in the next step without further purification.

### Step C. Procedure for Preparation of 3-[1-methyl-6-[(3R)-3-methylpiperazin-1-yl]indazol-3-yl]piperidine-2,6-dione

To a solution of tert-butyl (2R)-4-[3-(2,6-dioxo-3-piperidyl)-1-methyl-indazol-6-yl]-2-methyl-piperazine-1-carboxylate (630 mg, 1.43 mmol, 1 equiv.) in dioxane (8 mL) was added HCl/dioxane (4 M, 8 mL, 22.43 equiv.). The mixture was stirred at 25° C. for 2 hours. The reaction mixture was filtered to afford 3-[1-methyl-6-[(3R)-3-methylpiperazin-1-yl]indazol-3-yl]piperidine-2,6-dione (400 mg, crude) as a black brown solid, which was used in the next step without further purification.

### Step D. Procedure for Preparation of tert-butyl 6-[8-(1,3-benzothiazol-2-ylcarbamoyl)-3,4-dihydro-1H-isoquinolin-2-yl]-3-[3-[4-[3-[(2R)-4-[3-(2,6-dioxo-3-piperidyl)-1-methyl-indazol-6-yl]-2-methyl-piperazin-1-yl]propyl]cyclohexoxy]-2-methyl-phenyl]pyridine-2-carboxylate

To a solution of 3-[1-methyl-6-[(3R)-3-methylpiperazin-1-yl]indazol-3-yl]piperidine-2,6-dione (84.08 mg, 246.27 μmol, 1.2 equiv.) in DCM (2 mL) was added tert-butyl 6-[8-(1,3-benzothiazol-2-ylcarbamoyl)-3,4-dihydro-1H-isoquinolin-2-yl]-3-[2-methyl-3-[4-(3-oxopropyl)cyclohexoxy]phenyl]pyridine-2-carboxylate (150 mg, 205.22 μmol, 1 equiv.). The mixture was stirred at 25° C. for 1 hour, and then NaBH(OAc)₃ (130.49 mg, 615.67 μmol, 3 equiv.) was added and the mixture was stirred at 25° C. for 16 h. The reaction mixture was filtered and concentrated under vacuum to give a residue. The residue was purified by flash silica gel chromatography (eluent of 0-10% methanol/dichloromethane) to afford tert-butyl 6-[8-(1,3-benzothiazol-2-ylcarbamoyl)-3,4-dihydro-1H-isoquinolin-2-yl]-3-[3-[4-[3-[(2R)-4-[3-(2,6-dioxo-3-piperidyl)-1-methyl-indazol-6-yl]-2-methyl-piperazin-1-yl]propyl]cyclohexoxy]-2-methyl-phenyl]pyridine-2-carboxylate (80 mg, 72.7 μmol, 35.4% yield, 96% purity) as a light yellow solid.

MS (ESI) m/z: 1056.5 [M+H]⁺.

### Step E. Procedure for Preparation of 6-(8-(benzo[d]thiazol-2-ylcarbamoyl)-3,4-dihydroisoquinolin-2(1H)-yl)-3-(3-(((1S,4r)-4-(3-((2R)-4-(3-(2,6-dioxopiperidin-3-yl)-1-methyl-1H-indazol-6-yl)-2-methylpiperazin-1-yl)propyl)cyclohexyl)oxy)-2-methylphenyl)picolinic acid

To a solution of tert-butyl 6-[8-(1,3-benzothiazol-2-ylcarbamoyl)-3,4-dihydro-1H-isoquinolin-2-yl]-3-[3-[4-[3-[(2R)-4-[3-(2,6-dioxo-3-piperidyl)-1-methyl-indazol-6-yl]-2-methyl-piperazin-1-yl]propyl]cyclohexoxy]-2-methyl-phenyl]pyridine-2-carboxylate (70 mg, 66.27 μmol, 1 equiv.) in DCM (1 mL) was added TFA (1.54 g, 13.51 mmol, 1 mL, 203.82 equiv.). The mixture was stirred at 25° C. for 16 hours. The reaction mixture was concentrated under reduced pressure to give a residue. The residue was purified by prep-HPLC to afford 6-(8-(benzo[d]thiazol-2-ylcarbamoyl)-3,4-dihydroisoquinolin-2(1H)-yl)-3-(3-(((1S,4r)-4-(3-((2R)-4-(3-(2,6-dioxopiperidin-3-yl)-1-methyl-1H-indazol-6-yl)-2-methylpiperazin-1-yl)propyl)cyclohexyl)oxy)-2-methylphenyl)picolinic acid (27.8 mg, 27.6 μmol, 41.6% yield, 99.1% purity) as a white solid.

MS (ESI) m/z: 500.9 [M12+H]⁺.

¹H NMR (400 MHz, DMSO-d₆) δ=10.84 (s, 1H), 8.02 (d, J=7.2 Hz, 1H), 7.78 (d, J=8.0 Hz, 1H), 7.65-7.59 (m, 1H), 7.51-7.42 (m, 4H), 7.40-7.32 (m, 2H), 7.10-7.03 (m, 1H), 6.97-6.89 (m, 3H), 6.82 (s, 1H), 6.65-6.58 (m, 1H), 4.98 (s, 2H), 4.29-4.15 (m, 1H), 3.95-3.86 (m, 5H), 3.56-3.49 (m, 2H), 3.05-3.00 (m, 2H), 2.93-2.84 (m, 2H), 2.67 (s, 1H), 2.62-2.59 (m, 2H), 2.33-2.31 (m, 3H), 2.10-2.07 (m, 2H), 1.87 (s, 3H), 1.81-1.78 (m, 2H), 1.46-1.20 (m, 10H), 1.13-1.05 (m, 5H)

### Example 214. Preparation of Compound 278b

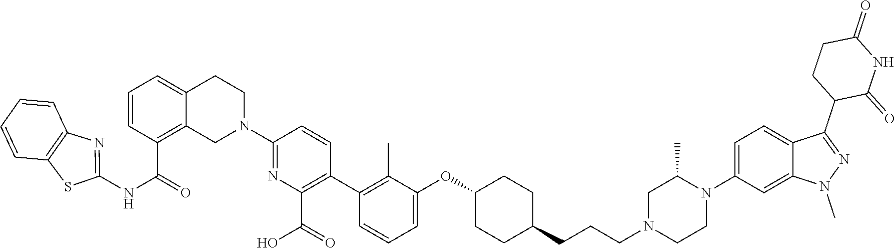 [SMILES: Cc1c(O[C@H]2CC[C@H](CCCN3CCN(c4ccc5c(C6CCC(=O)NC6=O)nn(C)c5c4)[C@@H](C)C3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1R,4r)-4-(3-((3S)-4-(3-(2,6-dioxopiperidin-3-yl)-1-methyl-1H-indazol-6-yl)-3-methylpiperazin-1-yl)propyl)cyclohexyl)oxy)-2-methylphenyl)picolinic acid

### Step A. Procedure for Preparation of tert-butyl (3S)-4-[3-(2,6-dibenzyloxy-3-pyridyl)-1-methyl-indazol-6-yl]-3-methyl-piperazine-1-carboxylate

A mixture of 6-bromo-3-(2,6-dibenzyloxy-3-pyridyl)-1-methyl-indazole (1.2 g, 2.40 mmol, 1.0 equiv.), tert-butyl (3S)-3-methylpiperazine-1-carboxylate (1.92 g, 9.59 mmol, 4.0 equiv.), Cs₂CO₃ (2.34 g, 7.19 mmol, 3.0 equiv.) and 1,3-bis[2,6-bis(1-propylbutyl)phenyl]-4,5-dichloro-2H-imidazol-1-ium-2-ide; 3-chloropyridine; dichloropalladium (200.00 mg, 205.60 μmol, 0.085 equiv.) in 2-methyl-2-butanol (30 mL) was degassed and purged with N₂ three times, and then the mixture was stirred at 100° C. for 16 hours under N₂ atmosphere. The mixture was filtered and concentrated under reduced pressure to give a residue. The residue was purified by flash silica gel chromatography (eluent of 0˜30% ethyl acetate/petroleum ether) to give tert-butyl (3S)-4-[3-(2,6-dibenzyloxy-3-pyridyl)-1-methyl-indazol-6-yl]-3-methyl-piperazine-1-carboxylate (1.2 g, 1.8 mmol, 76.8% yield, 95.2% purity) as a yellow oil.

¹H NMR (400 MHz, DMSO-d₆) δ=7.89 (d, J=8.0 Hz, 1H), 7.52 (d, J=9.2 Hz, 1H), 7.49-7.44 (m, 2H), 7.43-7.33 (m, 5H), 7.32-7.25 (m, 3H), 6.85-6.78 (m, 2H), 6.57 (d, J=8.0 Hz, 1H), 5.44 (s, 2H), 5.41 (s, 2H), 4.11-4.05 (m, 1H), 3.96 (s, 3H), 3.84-3.65 (m, 2H), 3.30 (d, J=12.0 Hz, 2H), 3.10-2.98 (m, 2H), 1.43 (s, 9H), 0.90 (d, J=6.4 Hz, 3H)

### Step B. Procedure for Preparation of tert-butyl (3S)-4-[3-(2,6-dioxo-3-piperidyl)-1-methyl-indazol-6-yl]-3-methyl-piperazine-1-carboxylate

To a solution of tert-butyl (3S)-4-[3-(2,6-dibenzyloxy-3-pyridyl)-1-methyl-indazol-6-yl]-3-methyl-piperazine-1-carboxylate (1.2 g, 1.94 mmol, 1.0 equiv.) in THE (5 mL) and EtOH (5 mL) was added Pd/C (300 mg, 10% purity), Pd(OH)₂ (300 mg, 2.14 mmol, 1.10 equiv.) and AcOH (11.63 mg, 193.63 μmol, 11.07 μL, 0.1 equiv.) under N₂ atmosphere. The suspension was degassed and purged with H₂ three times. The mixture was then stirred under H₂ (15 Psi) at 25° C. for 16 hours. The reaction mixture was filtered and washed with THE (50 mL). The filtrate was concentrated to give tert-butyl (3S)-4-[3-(2,6-dioxo-3-piperidyl)-1-methyl-indazol-6-yl]-3-methyl-piperazine-1-carboxylate (930 mg, 1.90 mmol, 97.9% yield, 90% purity) as a black brown solid.

### Step C. Procedure for Preparation of 3-[1-methyl-6-[(2S)-2-methylpiperazin-1-yl]indazol-3-yl]piperidine-2,6-dione

To a mixture of tert-butyl (3S)-4-[3-(2,6-dioxo-3-piperidyl)-1-methyl-indazol-6-yl]-3-methyl-piperazine-1-carboxylate (927.88 mg, 2.10 mmol, 1.0 equiv.) in DCM (5 mL) was added HCl/dioxane (4 M, 9.98 mL, 18.99 equiv.). The reaction was stirred at 25° C. for 2 hours. The reaction was then filtered to give 3-[1-methyl-6-[(2S)-2-methylpiperazin-1-yl]indazol-3-yl]piperidine-2,6-dione (600 mg, 1.4 mmol, 70.1% yield, 92.8% purity) as a white solid.

### Step D. Procedure for Preparation of tert-butyl 6-[8-(1,3-benzothiazol-2-ylcarbamoyl)-3,4-dihydro-1H-isoquinolin-2-yl]-3-[3-[4-[3-[(3S)-4-[3-(2,6-dioxo-3-piperidyl)-1-methyl-indazol-6-yl]-3-methyl-piperazin-1-yl]propyl]cyclohexoxy]-2-methyl-phenyl]pyridine-2-carboxylate

A mixture of 3-[1-methyl-6-[(2S)-2-methylpiperazin-1-yl]indazol-3-yl]piperidine-2,6-dione (270 mg, 714.54 μmol, 3.48 equiv.), tert-butyl 6-[8-(1,3-benzothiazol-2-ylcarbamoyl)-3,4-dihydro-1H-isoquinolin-2-yl]-3-[2-methyl-3-[4-(3-oxopropyl)cyclohexoxy]phenyl]pyridine-2-carboxylate (150 mg, 205.22 μmol, 1.0 equiv.), and NaBH(OAc)₃ (260.97 mg, 1.23 mmol, 6.0 equiv.) in DCM (2 mL) was stirred at 25° C. for 16 hours. The reaction mixture was filtered and concentrated under reduced pressure to give a residue. The residue was purified by prep-TLC (SiO₂, DCM:MeOH=15:1) to give tert-butyl 6-[8-(1,3-benzothiazol-2-ylcarbamoyl)-3,4-dihydro-1H-isoquinolin-2-yl]-3-[3-[4-[3-[(3S)-4-[3-(2,6-dioxo-3-piperidyl)-1-methyl-indazol-6-yl]-3-methyl-piperazin-1-yl]propyl]cyclohexoxy]-2-methyl-phenyl]pyridine-2-carboxylate (60 mg, 55.2 μmol, 26.9% yield, 97.3% purity) as a brown oil.

MS (ESI) m/z: 529.1 [M+H]⁺.

### Step E. Procedure for Preparation of 6-(8-(benzo[d]thiazol-2-ylcarbamoyl)-3,4-dihydroisoquinolin-2(1H)-yl)-3-(3-(((1R,4r)-4-(3-((3S)-4-(3-(2,6-dioxopiperidin-3-yl)-1-methyl-1H-indazol-6-yl)-3-methylpiperazin-1-yl)propyl)cyclohexyl)oxy)-2-methylphenyl)picolinic acid

To a solution of tert-butyl 6-[8-(1,3-benzothiazol-2-ylcarbamoyl)-3,4-dihydro-1H-isoquinolin-2-yl]-3-[3-[4-[3-[(3S)-4-[3-(2,6-dioxo-3-piperidyl)-1-methyl-indazol-6-yl]-3-methyl-piperazin-1-yl]propyl]cyclohexoxy]-2-methyl-phenyl]pyridine-2-carboxylate (60 mg, 56.80 μmol, 1 equiv.) in TFA (2 mL) and DCM (2 mL). The mixture was stirred at 25° C. for 12 hours. The mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1R,4r)-4-(3-((3S)-4-(3-(2,6-dioxopiperidin-3-yl)-1-methyl-1H-indazol-6-yl)-3-methylpiperazin-1-yl)propyl)cyclohexyl)oxy)-2-methylphenyl)picolinic acid (16.5 mg, 14.9 μmol, 26.2% yield, 89.0% purity) as a white solid.

MS (ESI) m/z: 500.8 [M/2+H]⁺.

¹H NMR (400 MHz, DMSO-d₆) δ=10.84 (s, 1H), 8.17 (s, 1H), 8.03 (d, J=7.6 Hz, 1H), 7.78 (d, J=8.0 Hz, 1H), 7.62 (d, J=7.2 Hz, 1H), 7.51-7.41 (m, 4H), 7.39-7.31 (m, 2H), 7.11-7.03 (m, 1H), 6.96-6.86 (m, 3H), 6.77 (s, 1H), 6.62 (d, J=7.2 Hz, 1H), 4.98 (s, 2H), 4.29-4.18 (m, 2H), 4.14-4.07 (m, 1H), 3.94-3.86 (m, 5H), 3.02 (m, 3H), 2.87 (d, J=10.4 Hz, 1H), 2.72 (d, J=10.0 Hz, 1H), 2.64-2.59 (m, 2H), 2.34-2.23 (m, 4H), 2.19-2.06 (m, 4H), 1.87 (s, 3H), 1.84-1.75 (m, 2H), 1.52-1.44 (m, 2H), 1.41-1.22 (m, 6H), 1.15-1.05 (m, 2H), 1.01 (d, J=6.4 Hz, 3H)

### Example 215. Preparation of Compound 266a

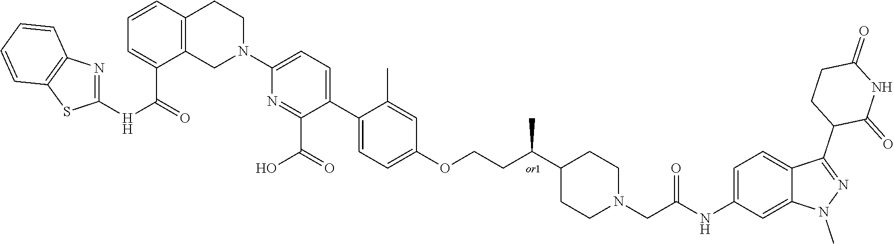

### 6-[8-(1,3-benzothiazol-2-ylcarbamoyl)-3,4-dihydro-1H-isoquinolin-2-yl]-3-[4-[(3R)-3-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylic acid

### [rel-6-(8-(benzo[d]thiazol-2-ylcarbamoyl)-3,4-dihydroisoquinolin-2(1H)-yl)-3-(4-((3R)-3-(1-(2-((3-(2,6-dioxopiperidin-3-yl)-1-methyl-1H-indazol-6-yl)amino)-2-oxoethyl)piperidin-4-yl)butoxy)-2-methylphenyl)picolinic acid]

### Step A. Procedure for Preparation of tert-butyl 4-[3-(4-bromo-3-methyl-phenoxy)-1-methyl-propyl]piperidine-1-carboxylate

To a solution of tert-butyl 4-(3-hydroxy-1-methyl-propyl)piperidine-1-carboxylate (2.2 g, 8.55 mmol, 1.0 equiv.) and 4-bromo-3-methyl-phenol (1.76 g, 9.40 mmol, 1.1 equiv.) in toluene (20 mL) was added 2-(tributyl-λ5-phosphanylidene)acetonitrile (2.48 g, 10.26 mmol, 1.2 equiv.). The mixture was stirred at 120° C. for 12 hours. The mixture was concentrated under reduced pressure to give a residue. The residue was purified by reverse-phase HPLC to give tert-butyl 4-[3-(4-bromo-3-methyl-phenoxy)-1-methyl-propyl]piperidine-1-carboxylate (2.7 g, 6.1 mmol, 72.1% yield, 97.4% purity) as a brown oil.

MS (ESI) m/z: 448.2 [M+23]⁺

¹H NMR (400 MHz, DMSO-d₆) δ=7.42 (d, J=8.8 Hz, 1H), 6.95 (d, J=2.8 Hz, 1H), 6.71 (dd, J=2.8, 8.8 Hz, 1H), 4.06-3.89 (m, 4H), 2.72-2.54 (m, 2H), 2.29 (s, 3H), 1.84-1.74 (m, 1H), 1.58-1.45 (m, 4H), 1.38 (s, 10H), 1.15-0.97 (m, 2H), 0.85 (d, J=6.4 Hz, 3H)

### Step B. Procedure for Preparation of tert-butyl 4-[(1S)-3-(4-bromo-3-methyl-phenoxy)-1-methyl-propyl]piperidine-1-carboxylate

The compound tert-butyl 4-[3-(4-bromo-3-methyl-phenoxy)-1-methyl-propyl]piperidine-1-carboxylate (1.4 g, 3.28 mmol, 1.0 equiv.) was purified by prep-HPLC to give tert-butyl 4-[(1R)-3-(4-bromo-3-methyl-phenoxy)-1-methyl-propyl]piperidine-1-carboxylate (430 mg, 1.00 mmol, 30.51% yield, 99.32% purity) as a brown oil and tert-butyl 4-[(1S)-3-(4-bromo-3-methyl-phenoxy)-1-methyl-propyl]piperidine-1-carboxylate (380 mg, 865.7 μmol, 26.3% yield, 97.1% purity) as a brown oil

MS (ESI) m/z: 448.2 [M+23]⁺

MS (ESI) m/z: 448.2 [M+23]⁺

### Step C. Procedure for Preparation of 4-[(1S)-3-(4-bromo-3-methyl-phenoxy)-1-methyl-propyl]piperidine

A mixture of tert-butyl 4-[(1S)-3-(4-bromo-3-methyl-phenoxy)-1-methyl-propyl]piperidine-1-carboxylate (430 mg, 1.01 mmol, 1.0 equiv.) and HCl/EtOAc (4 M, 5 mL, 19.83 equiv.) was stirred at 25° C. for 1 hour. The mixture was concentrated under reduced pressure to give 4-[(1R)-3-(4-bromo-3-methyl-phenoxy)-1-methyl-propyl]piperidine (380 mg, crude) as a brown solid.

MS (ESI) m/z: 328.2 [M+H]⁺(⁸⁰Br).

### Step D. Procedure for Preparation of ethyl 2-[4-[(1S)-3-(4-bromo-3-methyl-phenoxy)-1-methyl-propyl]-1-piperidyl]acetate

To a solution of 4-[(1R)-3-(4-bromo-3-methyl-phenoxy)-1-methyl-propyl]piperidine (380 mg, 1.05 mmol, 1.0 equiv., HCl) and ethyl 2-bromoacetate (157.46 mg, 942.85 μmol, 104.28 μL, 0.9 equiv.) in CH₃CN (4 mL) in DMF (0.5 mL) was added K₂CO₃ (434.36 mg, 3.14 mmol, 3.0 equiv.). The mixture was stirred at 60° C. for 1 hour. The mixture was then filtered to give a filtrate, which was concentrated under reduced pressure to give a residue. The residue was purified by column chromatography (SiO₂, petroleum ether/ethyl acetate=5/1 to 4/1) to give ethyl 2-[4-[(1R)-3-(4-bromo-3-methyl-phenoxy)-1-methyl-propyl]-1-piperidyl]acetate (370 mg, 892.1 μmol, 85.1% yield, 99.4% purity) as a light yellow oil.

MS (ESI) m/z: 414.2 [M+2+H]⁺(⁸⁰Br).

¹H NMR (400 MHz, DMSO-d₆) δ=7.42 (d, J=8.8 Hz, 1H), 6.95 (d, J=2.8 Hz, 1H), 6.71 (dd, J=2.8, 8.8 Hz, 1H), 4.07 (q, J=7.2 Hz, 2H), 4.03-3.90 (m, 2H), 3.14 (s, 2H), 2.88-2.80 (m, 2H), 2.29 (s, 3H), 2.13-2.04 (m, 2H), 1.84-1.75 (m, 1H), 1.56-1.43 (m, 4H), 1.33-1.21 (m, 2H), 1.21-1.15 (m, 4H), 0.86 (d, J=6.4 Hz, 3H)

### Step E. Procedure for Preparation of tert-butyl 6-[8-(1,3-benzothiazol-2-ylcarbamoyl)-3,4-dihydro-1H-isoquinolin-2-yl]-3-[4-[(3R)-3-[1-(2-ethoxy-2-oxo-ethyl)-4-piperidyl]butoxy]-2-methyl-phenyl]pyridine-2-carboxylate

Ethyl 2-[4-[(1R)-3-(4-bromo-3-methyl-phenoxy)-1-methyl-propyl]-1-piperidyl]acetate (150 mg, 363.76 μmol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (222.82 mg, 363.76 μmol, 1.0 equiv.), Ad₂nBuP Pd G₃ (26.49 mg, 36.38 μmol, 0.1 equiv.), and KF (63.40 mg, 1.09 mmol, 25.56 μL, 3.0 equiv.) were taken up into a microwave tube in dioxane (2 mL) and H₂O (0.2 mL). The sealed tube was heated at 100° C. for 1 hour under microwave. The mixture was concentrated under reduced pressure to give a residue. The residue was purified by column chromatography (SiO₂, petroleum ether/ethyl acetate=3/1 to 2/1) to give tert-butyl 6-[8-(1,3-benzothiazol-2-ylcarbamoyl)-3,4-dihydro-1H-isoquinolin-2-yl]-3-[4-[(3R)-3-[1-(2-ethoxy-2-oxo-ethyl)-4-piperidyl]butoxy]-2-methyl-phenyl]pyridine-2-carboxylate (170 mg, 179.1 μmol, 49.2% yield, 86.1% purity) as a yellow oil.

MS (ESI) m/z: 818.5 [M+H]⁺

¹H NMR (400 MHz, DMSO-d₆) δ=12.98-12.70 (m, 1H), 8.01 (d, J=7.6 Hz, 1H), 7.77 (d, J=8.0 Hz, 1H), 7.59 (d, J=7.6 Hz, 1H), 7.50-7.28 (m, 5H), 6.92 (d, J=8.8 Hz, 1H), 6.85 (d, J=8.4 Hz, 1H), 6.83-6.80 (m, 1H), 6.72 (dd, J=2.4, 8.4 Hz, 1H), 4.96 (s, 2H), 4.06 (q, J=6.8 Hz, 2H), 4.03-3.91 (m, 2H), 3.86 (t, J=6.0 Hz, 2H), 3.14 (s, 2H), 3.03 (t, J=6.0 Hz, 2H), 2.87-2.80 (m, 2H), 2.12-2.03 (m, 2H), 2.02-1.97 (m, 3H), 1.82-1.75 (m, 1H), 1.56-1.45 (m, 4H), 1.26-1.22 (m, 2H), 1.20-1.15 (m, 4H), 1.07-0.99 (m, 9H), 0.86 (d, J=6.4 Hz, 3H)

### Step F. Procedure for Preparation of 2-[4-[(1R)-3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]-1-methyl-propyl]-1-piperidyl]acetic acid

To a solution of tert-butyl 6-[8-(1,3-benzothiazol-2-ylcarbamoyl)-3,4-dihydro-1H-isoquinolin-2-yl]-3-[4-[(3R)-3-[1-(2-ethoxy-2-oxo-ethyl)-4-piperidyl]butoxy]-2-methyl-phenyl]pyridine-2-carboxylate (170 mg, 207.82 μmol, 1.0 equiv.) in THE (1.5 mL) and H₂O (0.5 mL) was added LiOH·H₂O (26.16 mg, 623.45 μmol, 3.0 equiv.). The mixture was stirred at 25° C. for 16 hours. The mixture was concentrated under reduced pressure to remove THF, diluted with H₂O (5 mL), added HCl (1 M) to pH=2, filtered, and concentrated under reduced pressure to give 2-[4-[(1R)-3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]-1-methyl-propyl]-1-piperidyl]acetic acid (160 mg, crude) as a yellow solid.

MS (ESI) m/z: 790.5 [M+H]⁺

¹H NMR (400 MHz, DMSO-d₆) δ=13.15-12.50 (m, 1H), 8.08-7.96 (m, 1H), 7.86-7.70 (m, 1H), 7.66-7.54 (m, 1H), 7.53-7.24 (m, 5H), 6.95-6.89 (m, 1H), 6.86 (d, J=8.0 Hz, 1H), 6.84-6.79 (m, 1H), 6.76-6.69 (m, 1H), 4.96 (s, 2H), 4.06-3.93 (m, 3H), 3.88-3.84 (m, 2H), 3.39-3.34 (m, 2H), 3.03 (t, J=5.6 Hz, 2H), 2.80-2.70 (m, 2H), 2.02-1.98 (m, 3H), 1.84-1.77 (m, 1H), 1.72-1.67 (m, 2H), 1.58-1.47 (m, 4H), 1.23 (s, 2H), 1.03 (s, 9H), 0.87 (d, J=6.0 Hz, 3H)

### Step G. Procedure for Preparation of tert-butyl 6-[8-(1,3-benzothiazol-2-ylcarbamoyl)-3,4-dihydro-1H-isoquinolin-2-yl]-3-[4-[(3R)-3-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylate

To a solution of 2-[4-[(1R)-3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]-1-methyl-propyl]-1-piperidyl]acetic acid (160 mg, 202.54 μmol, 1.0 equiv.) and HATU (92.41 mg, 243.04 μmol, 1.2 eq.) in DMF (1.5 mL) was added DIEA (78.53 mg, 607.61 μmol, 105.83 μL, 3.0 equiv.) and 3-(6-amino-1-methyl-indazol-3-yl)piperidine-2,6-dione (62.77 mg, 243.04 μmol, 1.2 equiv.). The mixture was stirred at 25° C. for 16 hours. The mixture was added into H₂O (10 mL), filtered, and concentrated under reduced pressure to give tert-butyl 6-[8-(1,3-benzothiazol-2-ylcarbamoyl)-3,4-dihydro-1H-isoquinolin-2-yl]-3-[4-[(3R)-3-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylate (140 mg, crude) as a pink solid.

MS (ESI) m/z: 1030.6 [M+H]⁺

¹H NMR (400 MHz, DMSO-d₆) δ=13.13-12.25 (m, 1H), 10.88 (s, 1H), 9.82 (s, 1H), 8.09-8.04 (m, 1H), 7.99 (d, J=8.0 Hz, 1H), 7.75 (d, J=8.0 Hz, 1H), 7.66-7.56 (m, 2H), 7.48-7.40 (m, 3H), 7.38-7.29 (m, 2H), 7.25-7.18 (m, 1H), 6.92 (d, J=8.8 Hz, 1H), 6.86 (d, J=8.4 Hz, 1H), 6.84-6.80 (m, 1H), 6.76-6.70 (m, 1H), 4.97 (s, 2H), 4.36-4.28 (m, 1H), 4.08-3.89 (m, 5H), 3.86 (t, J=5.6 Hz, 2H), 3.12 (s, 2H), 3.04-3.01 (m, 2H), 2.97-2.89 (m, 2H), 2.17-2.08 (m, 2H), 2.01 (s, 3H), 1.92-1.80 (m, 2H), 1.65-1.44 (m, 6H), 1.41-1.33 (m, 2H), 1.17 (s, 2H), 1.03 (s, 9H), 0.90 (d, J=6.8 Hz, 3H)

### Step H. Procedure for Preparation of 6-[8-(1,3-benzothiazol-2-ylcarbamoyl)-3,4-dihydro-1H-isoquinolin-2-yl]-3-[4-[(3R)-3-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylic acid [rel-6-(8-(benzo[d]thiazol-2-ylcarbamoyl)-3,4-dihydroisoquinolin-2(1H)-yl)-3-(4-((3R)-3-(1-(2-((3-(2,6-dioxopiperidin-3-yl)-1-methyl-1H-indazol-6-yl)amino)-2-oxoethyl)piperidin-4-yl)butoxy)-2-methylphenyl)picolinic acid]

To a solution of tert-butyl 6-[8-(1,3-benzothiazol-2-ylcarbamoyl)-3,4-dihydro-1H-isoquinolin-2-yl]-3-[4-[(3R)-3-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylate (140 mg, 135.89 μmol, 1.0 equiv.) in DCM (0.7 mL) was added TFA (1.08 g, 9.45 mmol, 0.7 mL, 69.57 equiv.). The mixture was stirred at 25° C. for 12 hours. The mixture was concentrated under reduced pressure to remove DCM. The mixture was purified by prep-HPLC to give 6-[8-(1,3-benzothiazol-2-ylcarbamoyl)-3,4-dihydro-1H-isoquinolin-2-yl]-3-[4-[(3R)-3-[1-[2-[[3-(2,6-dioxo-3-piperidyl)-1-methyl-indazol-6-yl]amino]-2-oxo-ethyl]-4-piperidyl]butoxy]-2-methyl-phenyl]pyridine-2-carboxylic acid [rel-6-(8-(benzo[d]thiazol-2-ylcarbamoyl)-3,4-dihydroisoquinolin-2(1H)-yl)-3-(4-((3R)-3-(1-(2-((3-(2,6-dioxopiperidin-3-yl)-1-methyl-1H-indazol-6-yl)amino)-2-oxoethyl)piperidin-4-yl)butoxy)-2-methylphenyl)picolinic acid] (26.1 mg, 24.8 μmol, 18.3% yield, 92.5% purity) as a yellow solid.

MS (ESI) m/z: 974.5 [M+H]⁺

¹H NMR (400 MHz, DMSO-d₆) δ=12.99-12.74 (m, 1H), 10.89 (s, 1H), 8.10-7.94 (m, 2H), 7.79 (d, J=8.0 Hz, 1H), 7.69-7.59 (m, 2H), 7.50-7.41 (m, 3H), 7.40-7.32 (m, 2H), 7.20-7.14 (m, 1H), 6.99-6.88 (m, 2H), 6.82-6.78 (m, 1H), 6.75-6.68 (m, 1H), 4.97 (s, 2H), 4.37-4.29 (m, 1H), 4.02-3.95 (m, 2H), 3.95-3.86 (m, 5H), 3.02 (t, J=5.6 Hz, 2H), 2.66-2.62 (m, 2H), 2.61-2.55 (m, 4H), 2.20-2.15 (m, 1H), 2.03 (s, 3H), 1.91-1.77 (m, 2H), 1.74-1.41 (m, 7H), 1.31-1.11 (m, 2H), 0.91 (d, J=6.4 Hz, 3H)

### Example 216. Preparation of Compound 126

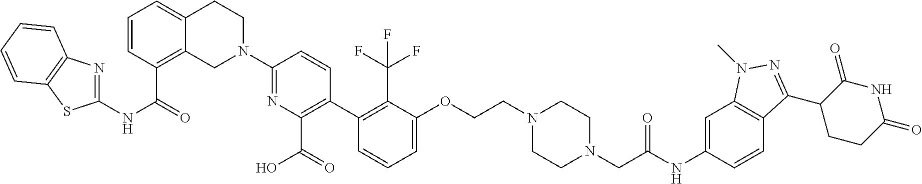 [SMILES: Cn1nc(C2CCC(=O)NC2=O)c2ccc(NC(=O)CN3CCN(CCOc4cccc(-c5ccc(N6CCc7cccc(C(=O)Nc8nc9ccccc9s8)c7C6)nc5C(=O)O)c4C(F)(F)F)CC3)cc21]

### 6-(8-(benzo[d]thiazol-2-ylcarbamoyl)-3,4-dihydroisoquinolin-2(1H)-yl)-3-(3-(2-(4-(2-((3-(2,6-dioxopiperidin-3-yl)-1-methyl-1H-indazol-6-yl)amino)-2-oxoethyl)piperazin-1-yl)ethoxy)-2-(trifluoromethyl)phenyl)picolinic acid

### Step A. Procedure for Preparation of 1-bromo-3-(2-bromoethoxy)-2-(trifluoromethyl)benzene

3-Bromo-2-(trifluoromethyl)phenol (601 mg, 1 equiv. 2.49 mmol), 1,2-dibromoethane (4.68 g, 2.15 mL, 10 equiv., 24.9 mmol), and potassium carbonate (431 mg, 1.25 equiv., 3.12 mmol) were combined in DMF (10 mL) and heated overnight at 50° C. The reaction mixture was diluted with ethyl acetate (100 mL) and brine (25 mL). The layers were separated, and the organic layers were dried over anhydrous magnesium sulfate, filtered, and concentrated to an oil, which was purified by silica gel chromatography (heptane/ethyl acetate gradient). The combined and concentrated product containing fractions afforded 1-bromo-3-(2-bromoethoxy)-2-(trifluoromethyl)benzene (602 mg, 1.73 mmol, 69.4% yield).

MS (ESI) m/z: 429.0 [M+H₂Br]+.

### Step B. Procedure for Preparation of methyl 2-(piperazin-1-yl)acetate hydrochloride

tert-Butyl 4-(2-methoxy-2-oxoethyl)piperazine-1-carboxylate (1.0 g, 1 equiv., 3.9 mmol) and HCl (0.71 g, 4.8 mL, 4 molar, 5 equiv., 19 mmol) (as a solution in dioxane) were combined in DCM (20 mL). The reaction was stirred overnight at ambient temperature. The reaction mixture was then concentrated to dryness, the solids were triturated in a mixture of diethyl ether and hexanes, and reconcentrated to provide methyl 2-(piperazin-1-yl)acetate hydrochloride (0.733 g, 3.77 mmol, 97% yield).

MS (ESI) m/z: 159.1 [M+H]⁺.

### Step C. Procedure for Preparation of methyl 2-(4-(2-(3-bromo-2-(trifluoromethyl)phenoxy)ethyl)piperazin-1-yl)acetate

Methyl 2-(piperazin-1-yl)acetate hydrochloride (281 mg, 1 equiv., 1.44 mmol), 1-bromo-3-(2-bromoethoxy)-2-(trifluoromethyl)benzene (602 mg, 1.2 equiv., 1.73 mmol), and potassium carbonate (598 mg, 3 equiv., 4.33 mmol) were combined in DMF (8 mL) and heated overnight at 50° C. The reaction mixture was diluted with ethyl acetate (100 mL) and brine (25 mL). The layers were separated, and the organic layer was dried over anhydrous magnesium sulfate, filtered, and concentrated to an oil, that was purified by silica gel chromatography (dichloromethane/methanol gradient). The combined and concentrated product containing fractions afforded methyl 2-(4-(2-(3-bromo-2-(trifluoromethyl)phenoxy)ethyl)piperazin-1-yl)acetate (398 mg, 936 μmol, 64.9% yield).

MS (ESI) m/z: 426.9 [M+H]+.

### Step D. Procedure for Preparation of methyl 2-(4-(2-(3-(4,4,5,5-tetramethyl-1,3,2-dioxaborolan-2-yl)-2-(trifluoromethyl)phenoxy)ethyl)piperazin-1-yl)acetate

Methyl 2-(4-(2-(3-bromo-2-(trifluoromethyl)phenoxy)ethyl)piperazin-1-yl)acetate (398 mg, 1 equiv., 936 μmol), bis(pinacolato)diboron (404 mg, 1.7 equiv., 1.59 mmol), and PdCl₂(dppf) (76.4 mg, 0.1 equiv., 93.6 μmol) were combined in 1,4-dioxane (5 mL). Potassium acetate (276 mg, 3 equiv., 2.81 mmol) was added and the reaction mixture stirred at 90° C. overnight. The reaction mixture was then diluted with 125 mL ethyl acetate, 25 mL water, and 25 mL brine. The layers were separated and the organic layer was washed with an additional 50 mL brine, dried over anhydrous magnesium sulfate, filtered, and concentrated to dryness under reduced pressure. The residue was purified on silica gel with hexanes/ethyl acetate. The product containing fractions were combined, concentrated, and dried under vacuum to afford methyl 2-(4-(2-(3-(4,4,5,5-tetramethyl-1,3,2-dioxaborolan-2-yl)-2-(trifluoromethyl)phenoxy)ethyl)piperazin-1-yl)acetate (328 mg, 694 μmol, 74.2% yield).

MS (ESI) m/z: 473.2 [M+H]⁺.

### Step E. Procedure for Preparation of tert-butyl 6-(8-(benzo[d]thiazol-2-ylcarbamoyl)-3,4-dihydroisoquinolin-2(1H)-yl)-3-(3-(2-(4-(2-methoxy-2-oxoethyl)piperazin-1-yl)ethoxy)-2-(trifluoromethyl)phenyl)picolinate

tert-Butyl 6-(8-(benzo[d]thiazol-2-ylcarbamoyl)-3,4-dihydroisoquinolin-2(1H)-yl)-3-bromopicolinate (327 mg, 1.0 equiv., 579 μmol) and methyl 2-(4-(2-(3-(4,4,5,5-tetramethyl-1,3,2-dioxaborolan-2-yl)-2-(trifluoromethyl)phenoxy)ethyl)piperazin-1-yl)acetate (328 mg, 1.2 equiv., 694 μmol) were combined in 1,4-dioxane (4.0 mL). An aqueous solution of potassium phosphate tribasic (369 mg, 1.16 mL, 1.5 molar, 3 equiv., 1.74 mmol) and cataCXium©Pd G3 (21.1 mg, 0.05 equiv., 28.9 μmol) was added. The reaction was stirred at 100° C. for 45 minutes using microwave heating and then cooled to ambient temperature. The reaction mixture was diluted with 125 mL ethyl acetate, 25 mL water, and 25 mL brine. The layers were separated and the organic layer was washed with an additional 50 mL brine, dried over anhydrous magnesium sulfate, filtered, and concentrated to dryness under reduced pressure. The residue was purified by silica gel chromatography (hexanes/ethyl acetate). The product containing fractions were combined and concentrated to afford tert-butyl 6-(8-(benzo[d]thiazol-2-ylcarbamoyl)-3,4-dihydroisoquinolin-2(1H)-yl)-3-(3-(2-(4-(2-methoxy-2-oxoethyl)piperazin-1-yl)ethoxy)-2-(trifluoromethyl)phenyl)picolinate (82 mg, 99 μmol, 17% yield).

MS (ESI) m/z: 831.3 [M+H]+.

### Step F. Procedure for Preparation of 2-(4-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-(trifluoromethyl)phenoxy)ethyl)piperazin-1-yl)acetic acid

tert-Butyl 6-(8-(benzo[d]thiazol-2-ylcarbamoyl)-3,4-dihydroisoquinolin-2(1H)-yl)-3-(3-(2-(4-(2-methoxy-2-oxoethyl)piperazin-1-yl)ethoxy)-2-(trifluoromethyl)phenyl)picolinate was dissolved in a mixture of THF (12 mL), methanol (3 mL), and water (3 mL). Lithium hydroxide (24 mg, 10 equiv., 0.99 mmol) was added and the reaction was stirred at ambient temperature overnight. The pH of the reaction was adjusted to ˜9-10 with a 1N aqueous solution of HCl and the volatiles were removed under reduced pressure. The residue was purified by reverse-phase HPLC (ACN/Water with 0.1% formic acid). The combined product containing fractions were lyophilized overnight to afford 2-(4-(2-(3-(6-(8-(benzo[d]thiazol-2-ylcarbamoyl)-3,4-dihydroisoquinolin-2(1H)-yl)-2-(tert-butoxycarbonyl)pyridin-3-yl)-2-(trifluoromethyl)phenoxy)ethyl)piperazin-1-yl)acetic acid (50 mg, 61 μmol, 62% yield).

MS (ESI) m/z: 817.1 [M+H]⁺.

### Step G. Procedure for Preparation of tert-butyl 6-(8-(benzo[d]thiazol-2-ylcarbamoyl)-3,4-dihydroisoquinolin-2(1H)-yl)-3-(3-(2-(4-(2-((3-(2,6-dioxopiperidin-3-yl)-1-methyl-1H-indazol-6-yl)amino)-2-oxoethyl)piperazin-1-yl)ethoxy)-2-(trifluoromethyl)phenyl)picolinate

2-(4-(2-(3-(6-(8-(Benzo[d]thiazol-2-ylcarbamoyl)-3,4-dihydroisoquinolin-2(1H)-yl)-2-(tert-butoxycarbonyl)pyridin-3-yl)-2-(trifluoromethyl)phenoxy)ethyl)piperazin-1-yl)acetic acid (50 mg, 1 equiv., 61 μmol) was dissolved in a mixture of DMF (5 mL) and DIEA (40 mg, 53 μL, 5 equiv., 0.31 mmol). HATU (28 mg, 1.2. equiv., 73 μmol) was added and the reaction was stirred at ambient temperature for a few minutes. 3-(6-amino-1-methyl-1H-indazol-3-yl)piperidine-2,6-dione (16 mg, 1 equiv., 61 μmol) was then added and the reaction was stirred overnight at ambient temperature. The reaction mixture was diluted with ethyl acetate (125 mL) and brine (30 mL). The layers were separated, and the organic layer dried over anhydrous magnesium sulfate, filtered, and concentrated to afford tert-butyl 6-(8-(benzo[d]thiazol-2-ylcarbamoyl)-3,4-dihydroisoquinolin-2(1H)-yl)-3-(3-(2-(4-(2-((3-(2,6-dioxopiperidin-3-yl)-1-methyl-1H-indazol-6-yl)amino)-2-oxoethyl)piperazin-1-yl)ethoxy)-2-(trifluoromethyl)phenyl)picolinate (65 mg, 61 μmol, quantitative yield assumed).

MS (ESI) m/z: 1057.0 [M+H]⁺.

### Step H. Procedure for Preparation of 6-(8-(benzo[d]thiazol-2-ylcarbamoyl)-3,4-dihydroisoquinolin-2(1H)-yl)-3-(3-(2-(4-(2-((3-(2,6-dioxopiperidin-3-yl)-1-methyl-1H-indazol-6-yl)amino)-2-oxoethyl)piperazin-1-yl)ethoxy)-2-(trifluoromethyl)phenyl)picolinic acid

tert-Butyl 6-(8-(benzo[d]thiazol-2-ylcarbamoyl)-3,4-dihydroisoquinolin-2(1H)-yl)-3-(3-(2-(4-(2-((3-(2,6-dioxopiperidin-3-yl)-1-methyl-1H-indazol-6-yl)amino)-2-oxoethyl)piperazin-1-yl)ethoxy)-2-(trifluoromethyl)phenyl)picolinate (65 mg, 1 equiv., 61 μmol) were dissolved in a mixture of DCM (10 mL) and trifluoroacetic acid (3.5 g, 2.4 mL, 500 equiv., 31 mmol). The reaction was stirred for about 60 hours at ambient temperature. The volatiles were removed under reduced pressure and the material purified by reverse-phase HPLC. The product containing fractions were lyophilized overnight to afford 6-(8-(benzo[d]thiazol-2-ylcarbamoyl)-3,4-dihydroisoquinolin-2(1H)-yl)-3-(3-(2-(4-(2-((3-(2,6-dioxopiperidin-3-yl)-1-methyl-1H-indazol-6-yl)amino)-2-oxoethyl)piperazin-1-yl)ethoxy)-2-(trifluoromethyl)phenyl)picolinic acid (40 mg, 40 μmol, 65% yield for two steps). ¹H NMR was consistent with structure.

MS (ESI) m/z: 501.2 [M+2H/2]⁺

### Example 217. Preparation of Compound 136

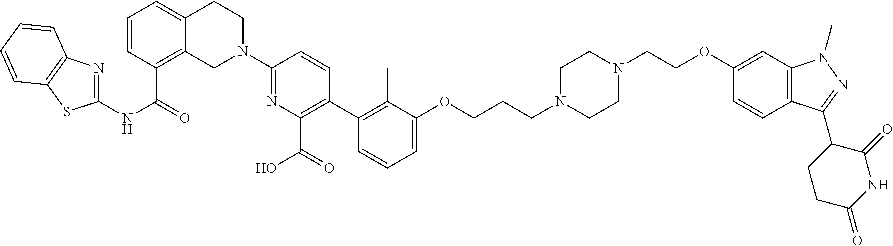 [SMILES: Cc1c(OCCCN2CCN(CCOc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-(4-(2-((3-(2,6-dioxopiperidin-3-yl)-1-methyl-1H-indazol-6-yl)oxy)ethyl)piperazin-1-yl)propoxy)-2-methylphenyl)picolinic acid

### Step A. Procedure for Preparation of (3-(3-bromo-2-methylphenoxy)propoxy)(tert-butyl)dimethylsilane

3-Bromo-2-methylphenol (2.00 g, 1 Eq, 10.7 mmol), (3-bromopropoxy)(tert-butyl)dimethylsilane (3.39 g, 1.25 equiv., 13.4 mmol), and potassium carbonate (2.22 g, 1.50 equiv., 16.0 mmol) were combined in DMF and heated overnight at 50° C. The reaction mixture was diluted with ethyl acetate (100 mL) and brine (25 mL). The layers were separated and the organic layers were dried over anhydrous magnesium sulfate, filtered, and concentrated to an oil that was purified by silica gel chromatography (heptane/ethyl acetate gradient). The combined and concentrated product containing fractions afforded (3-(3-bromo-2-methylphenoxy)propoxy)(tert-butyl)dimethylsilane (3.49 g, 9.71 mmol, 90.8% yield). ¹H NMR was consistent with structure.

### Step B. Procedure for Preparation of tert-butyldimethyl(3-(2-methyl-3-(4,4,5,5-tetramethyl-1,3,2-dioxaborolan-2-yl)phenoxy)propoxy)silane

Combined (3-(3-bromo-2-methylphenoxy)propoxy)(tert-butyl)dimethylsilane (1.00 g, 1 equiv., 2.78 mmol), bis(pinacolato)diboron (1.20 g, 1.7 equiv., 4.73 mmol), and PdCl₂(dppf) (227 mg, 0.1 equiv., 278 μmol) in 1,4-dioxane (15 mL). Potassium acetate (819 mg, 3 equiv., 8.35 mmol) was added and the mixture stirred at 90° C. overnight. The reaction mixture was diluted with 125 mL ethyl acetate, 25 mL water, and 25 mL brine. The layers were separated and the organic layer was washed with an additional 50 mL brine, dried over anhydrous magnesium sulfate, filtered, and concentrated to dryness under reduced pressure. The residue was purified by silica gel chromatography (hexanes/ethyl acetate). The product containing fractions were combined, concentrated, and dried under reduced pressure to afford tert-butyldimethyl(3-(2-methyl-3-(4,4,5,5-tetramethyl-1,3,2-dioxaborolan-2-yl)phenoxy)propoxy)silane (0.862 g, 2.12 mmol, 76.2% yield). ¹H NMR was consistent with structure.

MS (ESI) m/z: 407.4 [M+H]+.

### Step C. Procedure for Preparation of tert-butyl 6-(8-(benzo[d]thiazol-2-ylcarbamoyl)-3,4-dihydroisoquinolin-2(1H)-yl)-3-(3-(3-((tert-butyldimethylsilyl)oxy)propoxy)-2-methylphenyl)picolinate

tert-Butyl 6-(8-(benzo[d]thiazol-2-ylcarbamoyl)-3,4-dihydroisoquinolin-2(1H)-yl)-3-bromopicolinate (662 mg, 1 equiv., 1.17 mmol) and tert-butyldimethyl(3-(2-methyl-3-(4,4,5,5-tetramethyl-1,3,2-dioxaborolan-2-yl)phenoxy)propoxy)silane (571 mg, 1.2 equiv., 1.40 mmol) were combined in 1,4-dioxane (8.0 mL). Potassium phosphate (745 mg, 2.34 mL, 1.5 molar, 3 equiv., 3.51 mmol) and cataCXium© Pd G3 (42.6 mg, 0.05 equiv., 58.5 μmol) were added to the mixture. The reaction was stirred at 100° C. for 40 minutes in the microwave and then cooled to ambient temperature overnight. The reaction mixture was then diluted with 125 mL ethyl acetate, 25 mL water, and 25 mL brine. The layers were separated and the organic layer was washed with an additional 50 mL brine, dried over anhydrous magnesium sulfate, filtered, and concentrated to dryness under reduced pressure. The residue was purified by silica gel chromatography (hexanes/ethyl acetate). The product containing fractions were combined, concentrated, and dried under reduced pressure to afford tert-butyl 6-(8-(benzo[d]thiazol-2-ylcarbamoyl)-3,4-dihydroisoquinolin-2(1H)-yl)-3-(3-(3-((tert-butyldimethylsilyl)oxy)propoxy)-2-methylphenyl)picolinate (530 mg, 693 μmol, 59.2% yield).

MS (ESI) m/z: 765.3 [M+H]⁺.

### Step D. Procedure for Preparation of tert-butyl 6-(8-(benzo[d]thiazol-2-ylcarbamoyl)-3,4-dihydroisoquinolin-2(1H)-yl)-3-(3-(3-hydroxypropoxy)-2-methylphenyl)picolinate

TBAF (217 mg, 831 μL, 1.0 molar, 1.2 equiv., 831 μmol) as a 1N solution was combined in THE with tert-butyl 6-(8-(benzo[d]thiazol-2-ylcarbamoyl)-3,4-dihydroisoquinolin-2(1H)-yl)-3-(3-(3-((tert-butyldimethylsilyl)oxy)propoxy)-2-methylphenyl)picolinate (530 mg, 1 equiv., 693 μmol) dissolved in THE (10.0 mL). The reaction mixture was stirred at ambient temperature overnight. An additional portion of TBAF (217 mg, 831 μL, 1.0 molar, 1.2 equiv., 831 μmol) was added and the reaction was stirred again overnight at ambient temperature. The reaction mixture was then diluted with 125 mL ethyl acetate, 25 mL water, and 25 mL brine. The layers were separated and the organic layer was washed with an additional 50 mL brine, dried over anhydrous magnesium sulfate, filtered, and concentrated to dryness under reduced pressure. The residue was purified by silica gel (hexanes/ethyl acetate). The product containing fractions were combined, concentrated, triturated with a mixture of diethyl ether and hexanes, and re-concentrated to afford tert-butyl 6-(8-(benzo[d]thiazol-2-ylcarbamoyl)-3,4-dihydroisoquinolin-2(1H)-yl)-3-(3-(3-hydroxypropoxy)-2-methylphenyl)picolinate (322 mg, 495 μmol, 71.4% yield).

MS (ESI) m/z: 651.2 [M+H]⁺.

### Step E. Procedure for Preparation of tert-butyl 4-(2-((3-(2,6-bis(benzyloxy)pyridin-3-yl)-1-methyl-1H-indazol-6-yl)oxy)ethyl)piperazine-1-carboxylate

tert-Butyl 4-(2-hydroxyethyl)piperazine-1-carboxylate (920.5 mg, 2 equiv., 3.997 mmol) and 3-(2,6-bis(benzyloxy)pyridin-3-yl)-6-bromo-1-methyl-1H-indazole (1000 mg, 1 equiv., 1.998 mmol) were dissolved in dioxane (15 mL). To this solution tBuBrettPhos Pd G3 (85.38 mg, 0.05 equiv., 99.92 μmol) was added followed by potassium tert-butoxide (269.1 mg, 2.398 mL, 1.0 molar in THF, 1.2 equiv., 2.398 mmol). The reaction was then stirred at ambient temperature for about 60 hours. The reaction mixture was diluted with 125 mL ethyl acetate, 25 mL water, and 25 mL brine. The layers were separated and the organic layer was washed with an additional 50 mL brine, dried over anhydrous magnesium sulfate, filtered, and concentrated to dryness under reduced pressure. The residue was purified by silica gel chromatography (heptane/ethyl acetate containing 10% of a 7N solution of NH₃ in MeOH). The concentrated product containing fractions afforded tert-butyl 4-(2-((3-(2,6-bis(benzyloxy)pyridin-3-yl)-1-methyl-1H-indazol-6-yl)oxy)ethyl)piperazine-1-carboxylate (821 mg, 1.26 mmol, 63.2% yield).

MS (ESI) m/z: 650.3 [M+H]+.

### Step F. Procedure for Preparation of tert-butyl 4-(2-((3-(2,6-dioxopiperidin-3-yl)-1-methyl-1H-indazol-6-yl)oxy)ethyl)piperazine-1-carboxylate

tert-Butyl 4-(2-((3-(2,6-bis(benzyloxy)pyridin-3-yl)-1-methyl-1H-indazol-6-yl)oxy)ethyl)piperazine-1-carboxylate (821 mg, 1 equiv., 1.26 mmol) was dissolved in ethyl acetate (10 mL). Palladium on carbon (672 mg, 10% Wt, 0.25 equiv., 316 μmol) was added and the reaction was degassed under vacuum and then stirred at ambient temperature under an H₂ atmosphere overnight. The reaction was filtered through a pre-wetted pad of celite, concentrated to an oil under reduced pressure, and redissolved in ethyl acetate. Palladium on carbon (672 mg, 10% Wt, 0.25 eq, 316 μmol) was then added and the reaction was again degassed and stirred under a hydrogen atmosphere at ambient temperature for about 60 hours. The reaction was filtered through a pre-wetted pad of celite, and concentrated to an oil under reduced pressure to afford tert-butyl 4-(2-((3-(2,6-dioxopiperidin-3-yl)-1-methyl-1H-indazol-6-yl)oxy)ethyl)piperazine-1-carboxylate (596 mg, 1.26 mmol, quantitative yield assumed).

MS (ESI) m/z: 472.3 [M+H]⁺.

### Step G. Procedure for Preparation of 3-(1-methyl-6-(2-(piperazin-1-yl)ethoxy)-1H-indazol-3-yl)piperidine-2,6-dione hydrochloride

Tert-butyl 4-(2-((3-(2,6-dioxopiperidin-3-yl)-1-methyl-1H-indazol-6-yl)oxy)ethyl)piperazine-1-carboxylate (594 mg, 1 equiv., 914 μmol) was dissolved in DCM (50 mL), and 4N HCl in dioxane (667 mg, 4.57 mL, 4.0 molar, 20 equiv., 18.3 mmol) was added. The reaction mixture was stirred at ambient temperature overnight. The reaction was then concentrated under reduced pressure, and triturated in a mixture of diethyl ether and hexanes. The reconcentrated material afforded 3-(1-methyl-6-(2-(piperazin-1-yl)ethoxy)-1H-indazol-3-yl)piperidine-2,6-dione hydrochloride (306 mg, 750 μmol, 82.1% yield).

MS (ESI) m/z: 372.3 [M+H]+.

### Step H. Procedure for Preparation of tert-butyl 6-(8-(benzo[d]thiazol-2-ylcarbamoyl)-3,4-dihydroisoquinolin-2(1H)-yl)-3-(3-(3-chloropropoxy)-2-methylphenyl)picolinate

tert-Butyl 6-(8-(benzo[d]thiazol-2-ylcarbamoyl)-3,4-dihydroisoquinolin-2(1H)-yl)-3-(3-(3-hydroxypropoxy)-2-methylphenyl)picolinate (322 mg, 1 equiv., 495 μmol) was dissolved in DCM (10 mL). Thionyl chloride (118 mg, 72.2 μL, 2 equiv., 990 μmol) was added and the reaction mixture was stirred overnight at ambient temperature. The reaction mixture was diluted with additional DCM and a saturated aqueous solution of sodium bicarbonate. The layers were separated, and the organic layer dried over anhydrous magnesium sulfate, filtered, and concentrated to dryness under vacuum. The residue was purified by silica gel chromatography with hexanes/ethyl acetate. The product containing fractions were combined, concentrated, triturated with a mixture of diethyl ether and hexanes, and reconcentrated to afford tert-butyl 6-(8-(benzo[d]thiazol-2-ylcarbamoyl)-3,4-dihydroisoquinolin-2(1H)-yl)-3-(3-(3-chloropropoxy)-2-methylphenyl)picolinate (163 mg, 244 μmol, 49.2% yield).

MS (ESI) m/z: 669.1 [M+H]+.

### Step I. Procedure for preparation of tert-butyl 6-(8-(benzo[d]thiazol-2-ylcarbamoyl)-3,4-dihydroisoquinolin-2(1H)-yl)-3-(3-(3-(4-(2-((3-(2,6-dioxopiperidin-3-yl)-1-methyl-1H-indazol-6-yl)oxy)ethyl)piperazin-1-yl)propoxy)-2-methylphenyl)picolinate

tert-Butyl 6-(8-(benzo[d]thiazol-2-ylcarbamoyl)-3,4-dihydroisoquinolin-2(1H)-yl)-3-(3-(3-chloropropoxy)-2-methylphenyl)picolinate (75 mg, 1 equiv., 0.11 mmol) and 3-(1-methyl-6-(2-(piperazin-1-yl)ethoxy)-1H-indazol-3-yl)piperidine-2,6-dione hydrochloride (62 mg, 1.5 equiv., 0.17 mmol) were dissolved in DMF (0.5 mL). DIEA (72 mg, 98 μL, 5 equiv., 0.56 mmol) was added and the reaction was stirred at 60° C. overnight. An additional amount of DIEA (72 mg, 98 μL, 5 equiv., 0.56 mmol) was added and the reaction was stirred again overnight at 60° C. The reaction was purified by reverse-phase HPLC (ACN/water, with 0.1% formic acid). The concentrated product containing fractions afforded tert-butyl 6-(8-(benzo[d]thiazol-2-ylcarbamoyl)-3,4-dihydroisoquinolin-2(1H)-yl)-3-(3-(3-(4-(2-((3-(2,6-dioxopiperidin-3-yl)-1-methyl-1H-indazol-6-yl)oxy)ethyl)piperazin-1-yl)propoxy)-2-methylphenyl)picolinate (110 mg, 0.11 mmol, quantitative yield assumed).

MS (ESI) m/z: 1004.2 [M+H]+.

### Step J. Procedure for Preparation of 6-(8-(benzo[d]thiazol-2-ylcarbamoyl)-3,4-dihydroisoquinolin-2(1H)-yl)-3-(3-(3-(4-(2-((3-(2,6-dioxopiperidin-3-yl)-1-methyl-1H-indazol-6-yl)oxy)ethyl)piperazin-1-yl)propoxy)-2-methylphenyl)picolinic acid

tert-Butyl 6-(8-(benzo[d]thiazol-2-ylcarbamoyl)-3,4-dihydroisoquinolin-2(1H)-yl)-3-(3-(3-(4-(2-((3-(2,6-dioxopiperidin-3-yl)-1-methyl-1H-indazol-6-yl)oxy)ethyl)piperazin-1-yl)propoxy)-2-methylphenyl)picolinate (110 mg, 1 equiv., 110 μmol) was dissolved in a mixture of DCM (30 mL) and HCl in dioxane (2.00 g, 13.7 mL, 4.0 molar, 500 equiv., 54.8 mmol). The reaction was concentrated to dryness and the residue redissolved in DMSO. The material was purified by reverse-phase HPLC (ACN/water, with 0.1% formic acid). The product containing fractions were combined and lyophilized overnight to afford 6-(8-(benzo[d]thiazol-2-ylcarbamoyl)-3,4-dihydroisoquinolin-2(1H)-yl)-3-(3-(3-(4-(2-((3-(2,6-dioxopiperidin-3-yl)-1-methyl-1H-indazol-6-yl)oxy)ethyl)piperazin-1-yl)propoxy)-2-methylphenyl)picolinic acid (10.2 mg, 10.4 μmol, 9.46% yield over two steps). ¹H NMR was consistent with structure.

MS (ESI) m/z: 948.2 [M+H]⁺.

### Example 218. Preparation of Compound 165

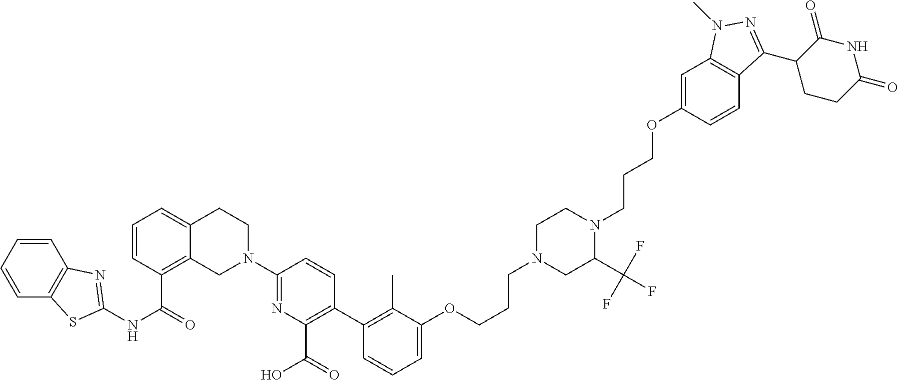 [SMILES: Cc1c(OCCCN2CCN(CCCOc3ccc4c(C5CCC(=O)NC5=O)nn(C)c4c3)C(C(F)(F)F)C2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-(4-(3-((3-(2,6-dioxopiperidin-3-yl)-1-methyl-1H-indazol-6-yl)oxy)propyl)-3-(trifluoromethyl)piperazin-1-yl)propoxy)-2-methylphenyl)picolinic acid

### Step A. Procedure for Preparation of tert-butyl 4-allyl-3-(trifluoromethyl)piperazine-1-carboxylate

tert-Butyl 3-(trifluoromethyl)piperazine-1-carboxylate (500 mg, 1 equiv., 1.97 mmol), allyl bromide (1.19 g, 851 μL, 5 equiv., 9.83 mmol), and DIEA (2.54 g, 3.43 mL, 10 equiv., 19.7 mmol) were combined in acetonitrile (8 mL) and heated overnight at 60° C. Added ½ the amounts of bromide and DIEA as originally used and continued heating at 60° C. for an additional night. The reaction mixture was diluted with ethyl acetate (100 mL) and brine (25 mL). The layers were separated, and the organic layers were over anhydrous magnesium sulfate, filtered, and concentrated to an oil that was purified by silica gel chromatography (heptane/ethyl acetate gradient). The combined and concentrated product containing fractions afforded tert-butyl 4-allyl-3-(trifluoromethyl)piperazine-1-carboxylate (533 mg, 1.81 mmol, 92.1% yield).

MS (ESI) m/z: 948.2 [M+H]⁺.

### Step B. Procedure for Preparation of tert-butyl 4-(3-hydroxypropyl)-3-(trifluoromethyl)piperazine-1-carboxylate

tert-Butyl 4-allyl-3-(trifluoromethyl)piperazine-1-carboxylate (400 mg, 1 equiv., 1.36 mmol) and 9-borabicyclo[3.3.1]nonane (199 mg, 3.26 mL, 0.5 molar, 1.2 equiv., 1.63 mmol) were combined in THE (10 mL) and stirred overnight at ambient temperature. Water (10 mL) was added to the reaction followed by sodium perborate tetrahydrate (627 mg, 3 equiv., 4.08 mmol) and the biphasic mixture was stirred for a few hours at ambient temperature. The reaction mixture was then diluted with ethyl acetate (125 mL), water (30 mL), and brine (30 mL). The layers were separated and the organic layer was dried over anhydrous magnesium sulfate, filtered and purified by silica gel chromatography (ethyl acetate/hexanes). The combined and concentrated product containing fractions afforded tert-butyl 4-(3-hydroxypropyl)-3-(trifluoromethyl)piperazine-1-carboxylate (371 mg, 1.19 mmol, 87.4% yield).

¹H NMR (400 MHz, DMSO) δ 4.37 (t, J=5.2 Hz, 1H), 3.97 (s, 1H), 3.82-3.52 (m, 2H), 3.49-3.36 (m, 2H), 3.24 (s, 1H), 3.05 (d, J=65.8 Hz, 1H), 2.85-2.60 (m, 4H), 1.57 (p, J=6.6 Hz, 2H), 1.38 (s, 9H).

### Step C. Procedure for Preparation of tert-butyl 4-(3-((3-(2,6-bis(benzyloxy)pyridin-3-yl)-1-methyl-1H-indazol-6-yl)oxy)propyl)-3-(trifluoromethyl)piperazine-1-carboxylate

tert-Butyl 4-(3-hydroxypropyl)-3-(trifluoromethyl)piperazine-1-carboxylate (370 mg, 2 equiv., 1.43 mmol) and 3-(2,6-bis(benzyloxy)pyridin-3-yl)-6-bromo-1-methyl-1H-indazole (358 mg, 1 equiv., 716 μmol) were dissolved in dioxane (10 mL). To this mixture tBuBrettPhos Pd G3 (30.6 mg, 0.05 equiv., 35.8 μmol) was added followed by potassium tert-butoxide (96.4 mg, 859 μL, 1.0 molar, 1.2 equiv., 859 μmol) and the reaction was stirred at ambient temperature overnight. The reaction was then diluted with ethyl acetate and brine. The layers were separated and the aqueous layers extracted with additional ethyl acetate. The combined organic layers were dried over anhydrous magnesium sulfate, filtered, and purified by silica gel chromatography (ethyl acetate/heptane). The combined product containing fractions were dried under vacuum over the weekend to afford tert-butyl 4-(3-((3-(2,6-bis(benzyloxy)pyridin-3-yl)-1-methyl-1H-indazol-6-yl)oxy)propyl)-3-(trifluoromethyl)piperazine-1-carboxylate (524 mg, quantitative yield assumed).

MS (ESI) m/z: 338.8 [M+2H/2]+.

### Step D. Procedure for Preparation of tert-butyl 4-(3-((3-(2,6-dioxopiperidin-3-yl)-1-methyl-1H-indazol-6-yl)oxy)propyl)-3-(trifluoromethyl)piperazine-1-carboxylate

tert-Butyl 4-(3-((3-(2,6-bis(benzyloxy)pyridin-3-yl)-1-methyl-1H-indazol-6-yl)oxy)propyl)-3-(trifluoromethyl)piperazine-1-carboxylate (433 mg, 1 equiv., 639 μmol) was dissolved in EtOAc (15 mL). To this solution Pd/C (340 mg, 10% Wt, 0.5 equiv., 319 μmol) was added. The resulting mixture was evacuated and backfilled with a balloon of hydrogen (3×). The mixture was stirred under a balloon of hydrogen at ambient temperature overnight. The reaction mixture was filtered through celite, and the filter cake was rinsed with ethyl acetate. The material was concentrated to dryness under reduced pressure and the residue purified by silica gel chromatography (ethyl acetate/hexanes) to afford tert-butyl 4-(3-((3-(2,6-dioxopiperidin-3-yl)-1-methyl-1H-indazol-6-yl)oxy)propyl)-3-(trifluoromethyl)piperazine-1-carboxylate (120 mg, 217 μmol, 33.9% yield).

MS (ESI) m/z: 554.3 [M+H]+.

### Step E. Procedure for Preparation of 3-(1-methyl-6-(3-(2-(trifluoromethyl)piperazin-1-yl)propoxy)-1H-indazol-3-yl)piperidine-2,6-dione hydrochloride

tert-Butyl 4-(3-((3-(2,6-dioxopiperidin-3-yl)-1-methyl-1H-indazol-6-yl)oxy)propyl)-3-(trifluoromethyl)piperazine-1-carboxylate (120 mg, 1 equiv., 217 μmol) was dissolved in DCM (10 mL). To this solution, HCl in dioxane (158 mg, 1.08 mL, 4.0 molar, 20 equiv., 4.34 mmol) was added and the reaction was stirred overnight at ambient temperature. The reaction was concentrated under reduced pressure to solids that were triturated with diethyl ether/hexanes, re-concentrated, and dried overnight under vacuum to afford 3-(1-methyl-6-(3-(2-(trifluoromethyl)piperazin-1-yl)propoxy)-1H-indazol-3-yl)piperidine-2,6-dione hydrochloride (136 mg, quantitative yield assumed).

MS (ESI) m/z: 454.1 [M+H]+.

### Step F. Procedure for Preparation of tert-butyl 6-(8-(benzo[d]thiazol-2-ylcarbamoyl)-3,4-dihydroisoquinolin-2(1H)-yl)-3-(3-(3-(4-(3-((3-(2,6-dioxopiperidin-3-yl)-1-methyl-1H-indazol-6-yl)oxy)propyl)-3-(trifluoromethyl)piperazin-1-yl)propoxy)-2-methylphenyl)picolinate

tert-Butyl 6-(8-(benzo[d]thiazol-2-ylcarbamoyl)-3,4-dihydroisoquinolin-2(1H)-yl)-3-(3-(3-chloropropoxy)-2-methylphenyl)picolinate (100 mg, 1.0 equiv., 149 μmol) and 3-(1-methyl-6-(3-(2-(trifluoromethyl)piperazin-1-yl)propoxy)-1H-indazol-3-yl)piperidine-2,6-dione hydrochloride (68 mg, 1.0 equiv., 0.15 mmol) were dissolved in NMP (1.0 mL). DIEA (97 mg, 0.13 mL, 5 equiv., 0.75 mmol) and sodium iodide (22 mg, 1 equiv., 0.15 mmol) were added and the reaction was stirred at 60° C. overnight. The reaction was purified by reverse-phase HPLC (ACN/water, with 0.1% formic acid). Product containing fractions were lyophilized to afford tert-butyl 6-(8-(benzo[d]thiazol-2-ylcarbamoyl)-3,4-dihydroisoquinolin-2(1H)-yl)-3-(3-(3-(4-(3-((3-(2,6-dioxopiperidin-3-yl)-1-methyl-1H-indazol-6-yl)oxy)propyl)-3-(trifluoromethyl)piperazin-1-yl)propoxy)-2-methylphenyl)picolinate (16 mg, 15 μmol, 9.8% yield).

MS (ESI) m/z: 544.0 [M+2H/2]+.

### Step G. Procedure for Preparation of 6-(8-(benzo[d]thiazol-2-ylcarbamoyl)-3,4-dihydroisoquinolin-2(1H)-yl)-3-(3-(3-(4-(3-((3-(2,6-dioxopiperidin-3-yl)-1-methyl-1H-indazol-6-yl)oxy)propyl)-3-(trifluoromethyl)piperazin-1-yl)propoxy)-2-methylphenyl)picolinic acid

tert-Butyl 6-(8-(benzo[d]thiazol-2-ylcarbamoyl)-3,4-dihydroisoquinolin-2(1H)-yl)-3-(3-(3-(4-(3-((3-(2,6-dioxopiperidin-3-yl)-1-methyl-1H-indazol-6-yl)oxy)propyl)-3-(trifluoromethyl)piperazin-1-yl)propoxy)-2-methylphenyl)picolinate (16 mg, 1 equiv., 15 μmol) was dissolved in DCM (5.0 mL). Trifluoroacetic acid (1.7 g, 1.1 mL, 1000 equiv., 15 mmol) was added and the reaction was stirred at ambient temperature overnight. The reaction mixture was concentrated to dryness under reduced pressure and the resulting residue was purified by reverse-phase HPLC (ACN/water, with 0.1% formic acid). The product containing fractions were combined and lyophilized to afford 6-(8-(benzo[d]thiazol-2-ylcarbamoyl)-3,4-dihydroisoquinolin-2(1H)-yl)-3-(3-(3-(4-(3-((3-(2,6-dioxopiperidin-3-yl)-1-methyl-1H-indazol-6-yl)oxy)propyl)-3-(trifluoromethyl)piperazin-1-yl)propoxy)-2-methylphenyl)picolinic acid (16.8 mg, 14.7 μmol, 100% yield). ¹H NMR was consistent with structure.

MS (ESI) m/z: 1030.4 [M+H]+.

### Example 219. Preparation of Compound 172

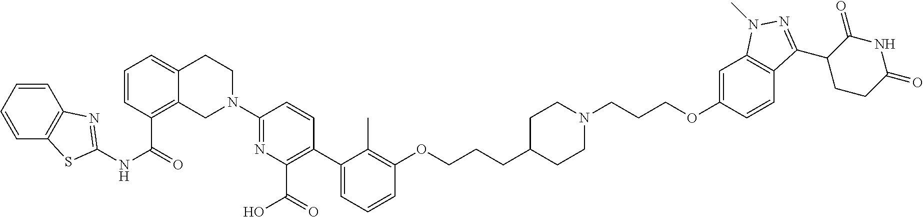 [SMILES: Cc1c(OCCCC2CCN(CCCOc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-(1-(3-((3-(2,6-dioxopiperidin-3-yl)-1-methyl-1H-indazol-6-yl)oxy)propyl)piperidin-4-yl)propoxy)-2-methylphenyl)picolinic acid

### Step A. Procedure for Preparation of 3-(2,6-bis(benzyloxy)pyridin-3-yl)-6-(3-((tert-butyldimethylsilyl)oxy)propoxy)-1-methyl-1H-indazole

Dissolved 3-((tert-butyldimethylsilyl)oxy)propan-1-ol (100 mg, 2 equiv., 525 μmol) and 3-(2,6-bis(benzyloxy)pyridin-3-yl)-6-bromo-1-methyl-1H-indazole (131 mg, 1 equiv., 263 μmol) in dioxane (4 mL). To this mixture was added tBuBrettPhos Pd G3 (11.2 mg, 0.05 equiv., 13.1 μmol) followed by potassium tert-butoxide (35.4 mg, 315 μL, 1.0 M in THF, 1.2 equiv., 315 μmol) and the reaction was allowed to mix at ambient temperature overnight. The reaction mixture was diluted with ethyl acetate and brine. The layers were separated and the extracted aqueous layers were washed with additional ethyl acetate. The combined organic layers were dried over anhydrous magnesium sulfate, filtered, and purified by silica gel chromatography (ethyl acetate/heptane). Product containing fractions were combined to afford 3-(2,6-bis(benzyloxy)pyridin-3-yl)-6-(3-((tert-butyldimethylsilyl)oxy)propoxy)-1-methyl-1H-indazole (160 mg, quantitative yield assumed). MS (ESI) m/z: 610.4 [M+H]⁺.

### Step B. Procedure for Preparation of 3-(6-(3-((tert-butyldimethylsilyl)oxy)propoxy)-1-methyl-1H-indazol-3-yl)piperidine-2,6-dione

3-(2,6-bis(benzyloxy)pyridin-3-yl)-6-(3-((tert-butyldimethylsilyl)oxy)propoxy)-1-methyl-1H-indazole (30 mg, 1 equiv., 49 μmol) was dissolved in ethyl acetate (10 mL). Palladium on carbon (52 mg, 10% Wt, 1 equiv., 49 μmol) was added and the reaction mixture was evacuated and back-filled with hydrogen (balloon). This process was repeated twice the mixture was stirred overnight at ambient temperature under a hydrogen atmosphere. The reaction mixture was then filtered through a bed of celite and concentrated to afford 3-(6-(3-((tert-butyldimethylsilyl)oxy)propoxy)-1-methyl-1H-indazol-3-yl)piperidine-2,6-dione (21 mg, quantitative yield assumed) that was used without further purification. MS (ESI) m/z: 432.3 [M+H]+.

### Step C. Procedure for Preparation of 3-(6-(3-hydroxypropoxy)-1-methyl-1H-indazol-3-yl)piperidine-2,6-dione

Dissolved 3-(6-(3-((tert-butyldimethylsilyl)oxy)propoxy)-1-methyl-1H-indazol-3-yl)piperidine-2,6-dione (363 mg, 1 equiv., 841 μmol) in THE (10 mL). Added TBAF (264 mg, 1.01 mL, 1.0 M in THF, 1.2 equiv., 1.01 mmol) and reaction was stirred overnight at ambient temperature. The reaction mixture was then concentrated to dryness under reduce pressure and purified by reverse-phase HPLC (ACN/water, with 0.1% formic acid). Pure product containing fractions were combined and concentrated under reduced pressure to afford 3-(6-(3-hydroxypropoxy)-1-methyl-1H-indazol-3-yl)piperidine-2,6-dione (115 mg, 0.36 mmol, 43.1% yield). MS (ESI) m/z: 318.0 [M+H]+.

### Step D. Procedure for Preparation of 3-(6-(3-chloropropoxy)-1-methyl-1H-indazol-3-yl)piperidine-2,6-dione

Dissolved 3-(6-(3-hydroxypropoxy)-1-methyl-1H-indazol-3-yl)piperidine-2,6-dione (143 mg, 1 equiv., 451 μmol) in DCM (10 mL). Added thionyl chloride (107 mg, 65.8 μL, 2 equiv., 901 μmol) and stirred reaction overnight at ambient temperature. An additional equivalent of thionyl chloride was added and the reaction was stirred overnight at ambient temperature. The reaction mixture was diluted with additional DCM and a saturated aqueous solution of sodium bicarbonate. The layers were separated, and the organic layer was dried over anhydrous magnesium sulfate, filtered, and concentrated to dryness under reduced pressure to afford 3-(6-(3-chloropropoxy)-1-methyl-1H-indazol-3-yl)piperidine-2,6-dione (143 mg, 451 μmol, 39.7% yield). MS (ESI) m/z: 336.1 [M+H]+.

### Step E. Procedure for Preparation of tert-butyl 6-(8-(benzo[d]thiazol-2-ylcarbamoyl)-3,4-dihydroisoquinolin-2(1H)-yl)-3-(3-(3-(1-(3-((3-(2,6-dioxopiperidin-3-yl)-1-methyl-1H-indazol-6-yl)oxy)propyl)piperidin-4-yl)propoxy)-2-methylphenyl)picolinate

Dissolved 3-(6-(3-chloropropoxy)-1-methyl-1H-indazol-3-yl)piperidine-2,6-dione (30 mg, 1 equiv., 89 μmol) and tert-butyl 6-(8-(benzo[d]thiazol-2-ylcarbamoyl)-3,4-dihydroisoquinolin-2(1H)-yl)-3-(2-methyl-3-(3-(piperidin-4-yl)propoxy)phenyl)picolinate (64 mg, 1 equiv., 89 μmol) in NMP (1 mL). Added DIEA (58 mg, 78 μL, 5 equiv., 0.45 mmol) and sodium iodide (13 mg, 1 equiv., 89 μmol) to the reaction mixture and reaction was stirred overnight at 60° C. The reaction mixture was purified by reversed-phase HPLC (ACN/water, with 0.1% formic acid. The combined and concentrated product containing fractions afforded tert-butyl 6-(8-(benzo[d]thiazol-2-ylcarbamoyl)-3,4-dihydroisoquinolin-2(1H)-yl)-3-(3-(3-(1-(3-((3-(2,6-dioxopiperidin-3-yl)-1-methyl-1H-indazol-6-yl)oxy)propyl)piperidin-4-yl)propoxy)-2-methylphenyl)picolinate (91 mg, quantitative yield assumed). MS (ESI) m/z: 1017.5 [M+H]⁺.

### Step F. Procedure for Preparation of 6-(8-(benzo[d]thiazol-2-ylcarbamoyl)-3,4-dihydroisoquinolin-2(1H)-yl)-3-(3-(3-(1-(3-((3-(2,6-dioxopiperidin-3-yl)-1-methyl-1H-indazol-6-yl)oxy)propyl)piperidin-4-yl)propoxy)-2-methylphenyl)picolinic acid

Dissolved tert-butyl 6-(8-(benzo[d]thiazol-2-ylcarbamoyl)-3,4-dihydroisoquinolin-2(1H)-yl)-3-(3-(3-(1-(3-((3-(2,6-dioxopiperidin-3-yl)-1-methyl-1H-indazol-6-yl)oxy)propyl)piperidin-4-yl)propoxy)-2-methylphenyl)picolinate (90 mg, 1 equiv., 88 μmol) in DCM (20 mL). Added a 4.0 M solution of hydrochloric acid in dioxane (0.16 g, 1.1 mL, 4.0 molar, 50 equiv., 4.4 mmol) and stirred reaction overnight at ambient temperature. The reaction was concentrated to dryness under reduced pressure, redissolved in a mixture of ACN and water, and lyophilized material to afford 6-(8-(benzo[d]thiazol-2-ylcarbamoyl)-3,4-dihydroisoquinolin-2(1H)-yl)-3-(3-(3-(1-(3-((3-(2,6-dioxopiperidin-3-yl)-1-methyl-1H-indazol-6-yl)oxy)propyl)piperidin-4-yl)propoxy)-2-methylphenyl)picolinic acid (10.6 mg, 10.7 μmol, 12% yield). ¹H NMR (400 MHz, DMSO-d₆) δ 12.87 (s, 1H), 10.87 (s, 1H), 9.49 (s, 1H), 8.04 (d, J=7.8 Hz, 1H), 7.80 (d, J=7.9 Hz, 1H), 7.61 (dd, J=17.5, 8.1 Hz, 2H), 7.52-7.42 (m, 4H), 7.41-7.32 (m, 2H), 7.19-7.05 (m, 2H), 6.98 (d, J=8.8 Hz, 1H), 6.89 (d, J=8.2 Hz, 1H), 6.82-6.70 (m, 1H), 6.64 (d, J=7.6 Hz, 1H), 4.99 (s, 2H), 4.31 (dd, J=9.3, 5.0 Hz, 1H), 4.23-4.08 (m, 2H), 4.03-3.86 (m, 7H), 3.22 (s, 2H), 2.94 (t, J=11.6 Hz, 2H), 2.77-2.58 (m, 2H), 2.26 (d, J=55.2 Hz, 4H), 1.83 (d, J=58.1 Hz, 10H), 1.57 (s, 1H), 1.54-1.29 (m, 5H), 1.24 (d, J=4.6 Hz, 3H). MS (ESI) m/z: 961.4 [M+H]⁺.

### Example 220. Preparation of Compound 189

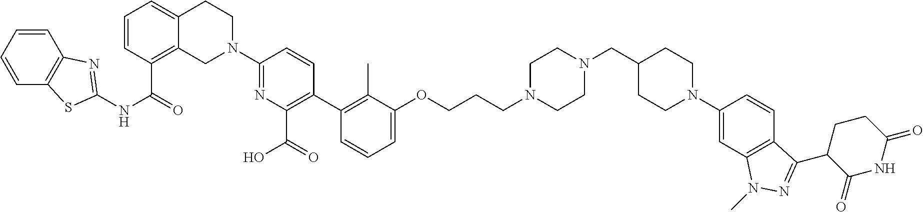 [SMILES: Cc1c(OCCCN2CCN(CC3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-(4-((1-(3-(2,6-dioxopiperidin-3-yl)-1-methyl-1H-indazol-6-yl)piperidin-4-yl)methyl)piperazin-1-yl)propoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(3-(4-((1-(3-(2,6-dioxopiperidin-3-yl)-1-methyl-1H-indazol-6-yl)piperidin-4-yl)methyl)piperazin-1-yl)propoxy)-2-methylphenyl)picolinate

tert-Butyl 6-(8-(benzo[d]thiazol-2-ylcarbamoyl)-3,4-dihydroisoquinolin-2(1H)-yl)-3-(3-(3-chloropropoxy)-2-methylphenyl)picolinate (83 mg, 1 equiv., 0.12 mmol) was dissolved in NMP (1 mL). To the mixture was added 3-(1-methyl-6-(4-(piperazin-1-ylmethyl)piperidin-1-yl)-1H-indazol-3-yl)piperidine-2,6-dione hydrochloride (57 mg, 1 equiv., 0.12 mmol) and sodium iodide (19 mg, 1 equiv., 0.12 mmol), followed by DIEA (80 mg, 0.11 mL, 5 equiv., 0.62 mmol). The mixture was then heated at 60° C. overnight. The reaction mixture was purified by reverse-phase HPLC (ACN/water, with 0.1% formic acid). Product containing fractions were combined and lyophilized to afford tert-butyl 6-(8-(benzo[d]thiazol-2-ylcarbamoyl)-3,4-dihydroisoquinolin-2(1H)-yl)-3-(3-(3-(4-((1-(3-(2,6-dioxopiperidin-3-yl)-1-methyl-1H-indazol-6-yl)piperidin-4-yl)methyl)piperazin-1-yl)propoxy)-2-methylphenyl)picolinate (40 mg, 0.038 mmol, 31% yield).

MS (ESI) m/z: 529.5 [M+2H/2]+.

### Step B. Procedure for Preparation of 6-(8-(benzo[d]thiazol-2-ylcarbamoyl)-3,4-dihydroisoquinolin-2(1H)-yl)-3-(3-(3-(4-((1-(3-(2,6-dioxopiperidin-3-yl)-1-methyl-1H-indazol-6-yl)piperidin-4-yl)methyl)piperazin-1-yl)propoxy)-2-methylphenyl)picolinic acid

tert-Butyl 6-(8-(benzo[d]thiazol-2-ylcarbamoyl)-3,4-dihydroisoquinolin-2(1H)-yl)-3-(3-(3-(4-((1-(3-(2,6-dioxopiperidin-3-yl)-1-methyl-1H-indazol-6-yl)piperidin-4-yl)methyl)piperazin-1-yl)propoxy)-2-methylphenyl)picolinate (40 mg, 1 equiv., 38 μmol) was dissolved in DCM (2.4 mL). To the mixture was added 0.6 mL TFA and the reaction mixture was then stirred at ambient temperature for two nights. The reaction mixture was concentrated under reduced pressure and the residue was resuspended in ACN/water and lyophilized to afford 6-(8-(benzo[d]thiazol-2-ylcarbamoyl)-3,4-dihydroisoquinolin-2(1H)-yl)-3-(3-(3-(4-((1-(3-(2,6-dioxopiperidin-3-yl)-1-methyl-1H-indazol-6-yl)piperidin-4-yl)methyl)piperazin-1-yl)propoxy)-2-methylphenyl)picolinic acid ditrifluoroacetate (51 mg, 41 μmol, quantitative yield). ¹H NMR was consistent with structure. MS (ESI) m/z: 1000.7 [M+H]+.

### Example 221. Preparation of Compound 192

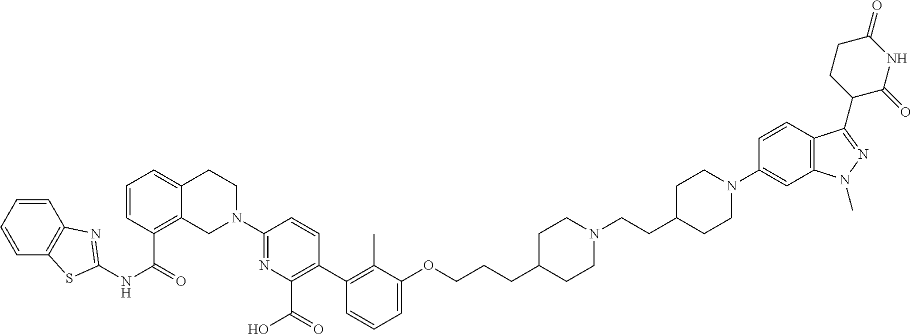 [SMILES: Cc1c(OCCCC2CCN(CCC3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-(1-(2-(1-(3-(2,6-dioxopiperidin-3-yl)-1-methyl-1H-indazol-6-yl)piperidin-4-yl)ethyl)piperidin-4-yl)propoxy)-2-methylphenyl)picolinic acid

### Step A. Procedure for Preparation of 6-(4-(2-(benzyloxy)ethyl)piperidin-1-yl)-3-(2,6-bis(benzyloxy)pyridin-3-yl)-1-methyl-1H-indazole

3-(2,6-Bis(benzyloxy)pyridin-3-yl)-6-bromo-1-methyl-1H-indazole (1.0 g, 1 equiv., 2.0 mmol) was suspended in 1,4-dioxane (15 mL). To the mixture was added 4-(2-(benzyloxy)ethyl)piperidine hydrochloride (0.61 g, 1.2 equiv., 2.4 mmol), followed by (2-dicyclohexylphosphino-2′,6′-diisopropoxy-1,1′-biphenyl)[2-(2′-amino-1,1′-biphenyl)]palladium(II) methanesulfonate (RuPhos Pd G3, 0.17 g, 0.1 eq, 0.20 mmol), and sodium 2-methylpropan-2-olate (0.58 g, 3.0 mL, 2.0 M in THF, 3 equiv., 6.0 mmol). The mixture was then purged with N₂ and heated at 100° C. for 2 h. The mixture was cooled and then diluted with ethyl acetate and water. The organic layer was separated, washed with brine, dried over anhydrous sodium sulfate, filtered, and concentrated under reduced pressure to dryness. The residue was purified by RP-HPLC. The desired fractions were combined and concentrated under reduced pressure to afford 6-(4-(2-(benzyloxy)ethyl)piperidin-1-yl)-3-(2,6-bis(benzyloxy)pyridin-3-yl)-1-methyl-1H-indazole (0.65 g, 1.0 mmol, 51% yield). MS (ESI) m/z: 661.5 [M+Na]⁺.

### Step B. Procedure for Preparation of 3-(6-(4-(2-hydroxyethyl)piperidin-1-yl)-1-methyl-11H-indazol-3-yl)piperidine-2,6-dione

6-(4-(2-(Benzyloxy)ethyl)piperidin-1-yl)-3-(2,6-bis(benzyloxy)pyridin-3-yl)-1-methyl-1H-indazole (650 mg, 1 equiv., 1.02 mmol) was dissolved in ethyl acetate (15 mL). To the mixture was added Pd/C (541 mg, 10% Wt, 0.5 equiv., 509 μmol). The mixture was evacuated and backfilled with H₂ (3×). The mixture was then stirred at ambient temperature under a H₂ atmosphere (balloon pressure). The reaction mixture was filtered and the filter cake was washed with ethyl acetate. The filtrate was concentrated under reduced pressure and the resulting crude material was then purified by reverse-phase HPLC (ACN/water, with 0.1% formic acid) to afford 3-(6-(4-(2-hydroxyethyl)piperidin-1-yl)-1-methyl-1H-indazol-3-yl)piperidine-2,6-dione (101 mg, 273 μmol, 26.8%). ¹H NMR was consistent with structure. MS (ESI) m/z: 371.3 [M+H]+.

### Step C. Procedure for Preparation of 2-(1-(3-(2,6-dioxopiperidin-3-yl)-1-methyl-1H-indazol-6-yl)piperidin-4-yl)acetaldehyde

3-(6-(4-(2-Hydroxyethyl)piperidin-1-yl)-1-methyl-1H-indazol-3-yl)piperidine-2,6-dione (40 mg, 1 equiv., 0.11 mmol) was dissolved in DCM (4 mL). To the mixture was added Dess-Martin periodinane (50 mg, 37 μL, 1.1 equiv., 0.12 mmol). The mixture was then stirred at ambient temperature for ˜1 h. A few drops methanol was added to the reaction mixture and it was then concentrated under reduced pressure to afford 2-(1-(3-(2,6-dioxopiperidin-3-yl)-1-methyl-1H-indazol-6-yl)piperidin-4-yl)acetaldehyde (40 mg, quantitative yield assumed) as a crude material that was used without further purification. MS (ESI) m/z: 369.3 [M+H]+.

### Step D. Procedure for Preparation of tert-butyl 6-(8-(benzo[d]thiazol-2-ylcarbamoyl)-3,4-dihydroisoquinolin-2(1H)-yl)-3-(3-(3-(1-(2-(1-(3-(2,6-dioxopiperidin-3-yl)-1-methyl-1H-indazol-6-yl)piperidin-4-yl)ethyl)piperidin-4-yl)propoxy)-2-methylphenyl)picolinate

tert-Butyl 6-(8-(benzo[d]thiazol-2-ylcarbamoyl)-3,4-dihydroisoquinolin-2(1H)-yl)-3-(2-methyl-3-(3-(piperidin-4-yl)propoxy)phenyl)picolinate (60 mg, 1 equiv., 84 μmol) and 2-(1-(3-(2,6-dioxopiperidin-3-yl)-1-methyl-1H-indazol-6-yl)piperidin-4-yl)acetaldehyde (40 mg, 1.3 equiv., 0.11 mmol) were suspended in ethanol (8 mL). To the mixture was added sodium cyanoborohydride (26 mg, 5 equiv., 0.42 mmol), followed by acetic acid (15 mg, 14 μL, 3 equiv., 0.25 mmol). The mixture was then stirred at ambient temperature overnight. The mixture was evaporated under reduced pressure and the residue was then purified by reverse-phase HPLC (ACN/water, with 0.1% formic acid) to afford tert-butyl 6-(8-(benzo[d]thiazol-2-ylcarbamoyl)-3,4-dihydroisoquinolin-2(1H)-yl)-3-(3-(3-(1-(2-(1-(3-(2,6-dioxopiperidin-3-yl)-1-methyl-1H-indazol-6-yl)piperidin-4-yl)ethyl)piperidin-4-yl)propoxy)-2-methylphenyl)picolinate (51 mg, 48 μmol, 57% yield). MS (ESI) m/z: 536.0 [M+2H/2]+.

### Step E. Procedure for Preparation of 6-(8-(benzo[d]thiazol-2-ylcarbamoyl)-3,4-dihydroisoquinolin-2(1H)-yl)-3-(3-(3-(1-(2-(1-(3-(2,6-dioxopiperidin-3-yl)-1-methyl-1H-indazol-6-yl)piperidin-4-yl)ethyl)piperidin-4-yl)propoxy)-2-methylphenyl)picolinic acid

tert-Butyl 6-(8-(benzo[d]thiazol-2-ylcarbamoyl)-3,4-dihydroisoquinolin-2(1H)-yl)-3-(3-(3-(1-(2-(1-(3-(2,6-dioxopiperidin-3-yl)-1-methyl-1H-indazol-6-yl)piperidin-4-yl)ethyl)piperidin-4-yl)propoxy)-2-methylphenyl)picolinate (30 mg, 1 equiv., 28 μmol) was dissolved in DCM (2.4 mL). To the mixture was added 0.6 mL TFA. The mixture was then stirred at ambient temperature for two nights. The solvents were removed under reduced pressure and the residue was purified by reverse-phase HPLC (ACN/water, with 0.1% formic acid). The desired fractions were combined and concentrated under reduced pressure to afford 6-(8-(benzo[d]thiazol-2-ylcarbamoyl)-3,4-dihydroisoquinolin-2(1H)-yl)-3-(3-(3-(1-(2-(1-(3-(2,6-dioxopiperidin-3-yl)-1-methyl-1H-indazol-6-yl)piperidin-4-yl)ethyl)piperidin-4-yl)propoxy)-2-methylphenyl)picolinic acid (24 mg, 24 μmol, 84% yield). ¹H NMR was consistent with structure. MS (ESI) m/z: 1014.8 [M+H]+.

### Example 222. Preparation of Compound 198

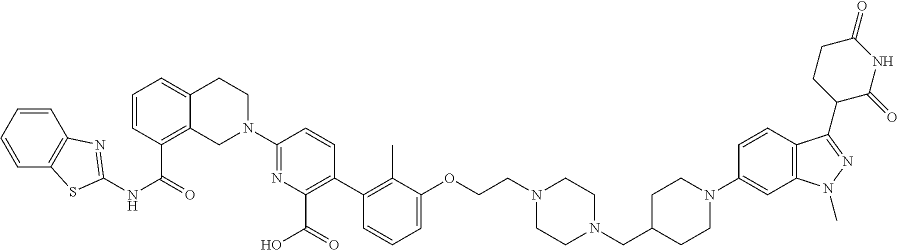 [SMILES: Cc1c(OCCN2CCN(CC3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(2-(4-((1-(3-(2,6-dioxopiperidin-3-yl)-1-methyl-1H-indazol-6-yl)piperidin-4-yl)methyl)piperazin-1-yl)ethoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(2-(4-((1-(3-(2,6-dioxopiperidin-3-yl)-1-methyl-1H-indazol-6-yl)piperidin-4-yl)methyl)piperazin-1-yl)ethoxy)-2-methylphenyl)picolinate

tert-Butyl 6-(8-(benzo[d]thiazol-2-ylcarbamoyl)-3,4-dihydroisoquinolin-2(1H)-yl)-3-(3-(2-chloroethoxy)-2-methylphenyl)picolinate (100 mg, 1 equiv., 153 μmol), 3-(1-methyl-6-(4-(piperazin-1-ylmethyl)piperidin-1-yl)-1H-indazol-3-yl)piperidine-2,6-dione hydrochloride (70.4 mg, 1 equiv., 153 μmol), and sodium iodide (27.5 mg, 1.2 equiv., 183 μmol) were combined. To this mixture was added DIEA (98.6 mg, 133 μL, 5 equiv., 763 μmol). The resulting mixture was heated at 60° C. overnight, cooled, and then purified directly by reverse-phase HPLC (ACN/water, with 0.1% formic acid) to afford tert-butyl 6-(8-(benzo[d]thiazol-2-ylcarbamoyl)-3,4-dihydroisoquinolin-2(1H)-yl)-3-(3-(2-(4-((1-(3-(2,6-dioxopiperidin-3-yl)-1-methyl-1H-indazol-6-yl)piperidin-4-yl)methyl)piperazin-1-yl)ethoxy)-2-methylphenyl)picolinate (33 mg, 32 μmol, 21% yield). MS (ESI) m/z: 522.5 [M+2H/2]+.

### Step B. Procedure for Preparation of 6-(8-(benzo[d]thiazol-2-ylcarbamoyl)-3,4-dihydroisoquinolin-2(1H)-yl)-3-(3-(2-(4-((1-(3-(2,6-dioxopiperidin-3-yl)-1-methyl-1H-indazol-6-yl)piperidin-4-yl)methyl)piperazin-1-yl)ethoxy)-2-methylphenyl)picolinic acid

tert-Butyl 6-(8-(benzo[d]thiazol-2-ylcarbamoyl)-3,4-dihydroisoquinolin-2(1H)-yl)-3-(3-(2-(4-((1-(3-(2,6-dioxopiperidin-3-yl)-1-methyl-1H-indazol-6-yl)piperidin-4-yl)methyl)piperazin-1-yl)ethoxy)-2-methylphenyl)picolinate (33 mg, 1 equiv., 32 μmol)) was dissolved in DCM (2.4 mL). To the mixture was added 0.6 mL TFA. The mixture was then stirred at ambient temperature for two nights. The reaction was concentrated under reduced pressure, resuspended in ACN/water, and lyophilized overnight to afford 6-(8-(benzo[d]thiazol-2-ylcarbamoyl)-3,4-dihydroisoquinolin-2(1H)-yl)-3-(3-(2-(4-((1-(3-(2,6-dioxopiperidin-3-yl)-1-methyl-1H-indazol-6-yl)piperidin-4-yl)methyl)piperazin-1-yl)ethoxy)-2-methylphenyl)picolinic acid difluoroacetate (40 mg, 33 μmol, quantitative yield). ¹H NMR was consistent with structure. MS (ESI) m/z: 987.6 [M+H]+.

### Example 223. Preparation of Compound 203

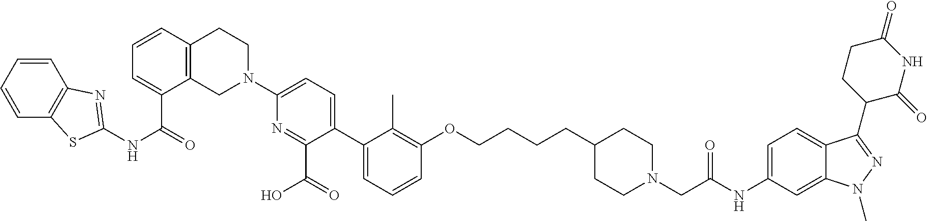 [SMILES: Cc1c(OCCCCC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(4-(1-(2-((3-(2,6-dioxopiperidin-3-yl)-1-methyl-1H-indazol-6-yl)amino)-2-oxoethyl)piperidin-4-yl)butoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(4-(1-(2-((3-(2,6-dioxopiperidin-3-yl)-1-methyl-1H-indazol-6-yl)amino)-2-oxoethyl)piperidin-4-yl)butoxy)-2-methylphenyl)picolinate

To a solution of 2-(4-(4-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)butyl)piperidin-1-yl)acetic acid (60 mg, 1 equiv., 76 μmol) in DCM (5 mL) was added HATU (32 mg, 1.1 equiv., 84 μmol), followed by 3-(6-amino-1-methyl-1H-indazol-3-yl)piperidine-2,6-dione (22 mg, 1.1 equiv., 84 μmol) and diisopropylethylamine (29 mg, 40 μL, 3 equiv., 0.23 mmol). The reaction solution was then stirred at room temperature. After 16 h, the reaction solution was filtered and concentrated to provide crude tert-butyl 6-(8-(benzo[d]thiazol-2-ylcarbamoyl)-3,4-dihydroisoquinolin-2(1H)-yl)-3-(3-(4-(1-(2-((3-(2,6-dioxopiperidin-3-yl)-1-methyl-1H-indazol-6-yl)amino)-2-oxoethyl)piperidin-4-yl)butoxy)-2-methylphenyl)picolinate, which was carried forward without further purification. MS (ESI) m/z: 1029.6 [M+H]⁺.

### Step B. Procedure for Preparation of 6-(8-(benzo[d]thiazol-2-ylcarbamoyl)-3,4-dihydroisoquinolin-2(1H)-yl)-3-(3-(4-(1-(2-((3-(2,6-dioxopiperidin-3-yl)-1-methyl-1H-indazol-6-yl)amino)-2-oxoethyl)piperidin-4-yl)butoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(4-(1-(2-((3-(2,6-dioxopiperidin-3-yl)-1-methyl-1H-indazol-6-yl)amino)-2-oxoethyl)piperidin-4-yl)butoxy)-2-methylphenyl)picolinate (78 mg, 76 μmol, 1 equiv.) in DCM (4 mL) was added trifluoroacetic acid (1 mL), and the reaction solution was stirred at room temperature. After 16 h, the reaction solution was concentrated, dissolved in DMSO, and purified by reverse-phase HPLC to provide 6-(8-(benzo[d]thiazol-2-ylcarbamoyl)-3,4-dihydroisoquinolin-2(1H)-yl)-3-(3-(4-(1-(2-((3-(2,6-dioxopiperidin-3-yl)-1-methyl-1H-indazol-6-yl)amino)-2-oxoethyl)piperidin-4-yl)butoxy)-2-methylphenyl)picolinic acid (30 mg, 31 μmol, 41% yield) as a white solid. MS (ESI) m/z: 974 [M+H]⁺. ¹H NMR is consistent with structure.

### Example 224. Preparation of Compound 236

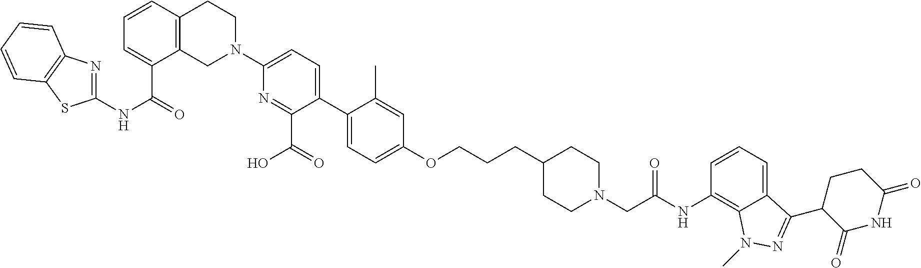 [SMILES: Cc1cc(OCCCC2CCN(CC(=O)Nc3cccc4c(C5CCC(=O)NC5=O)nn(C)c34)CC2)ccc1-c1ccc(N2CCc3cccc(C(=O)Nc4nc5ccccc5s4)c3C2)nc1C(=O)O]

### 6-(8-(Benzo[d]thiazol-2-ylcarbamoyl)-3,4-dihydroisoquinolin-2(1H)-yl)-3-(4-(3-(1-(2-((3-(2,6-dioxopiperidin-3-yl)-1-methyl-1H-indazol-7-yl)amino)-2-oxoethyl)piperidin-4-yl)propoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-1-methyl-1H-indazol-7-yl)amino)-2-oxoethyl)piperidin-4-yl)propoxy)-2-methylphenyl)picolinate

A mixture of 2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)propyl)piperidin-1-yl)acetic acid (37 mg, 1 equiv, 48 μmol), 3-(7-amino-1-methyl-1H-indazol-3-yl)piperidine-2,6-dione (15 mg, 1.2 equiv, 57 μmol), N,N,N′,N′-tetramethylchloroformamidinium-hexafluorophosphate (16 mg, 1.2 equiv, 57 μmol) and 1-methylimidazole (14 mg, 13 μL, 3.5 equiv., 0.17 mmol) was suspended in MeCN (5 mL) and the reaction solution was stirred at room temperature. After 16 h, the reaction solution was concentrated, re-suspended in ethyl acetate and water, and extracted with ethyl acetate. The combined organic layers were washed with brine, dried over anhydrous sodium sulfate and concentrated to provide tert-butyl 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-1-methyl-1H-indazol-7-yl)amino)-2-oxoethyl)piperidin-4-yl)propoxy)-2-methylphenyl)picolinate, which was carried forward without further purification. MS (ESI) m/z: 1016.6 [M+H]⁺.

### Step B. Procedure for Preparation of 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-1-methyl-1H-indazol-7-yl)amino)-2-oxoethyl)piperidin-4-yl)propoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-1-methyl-1H-indazol-7-yl)amino)-2-oxoethyl)piperidin-4-yl)propoxy)-2-methylphenyl)picolinate (49 mg, 48 μmol, 1 equiv.) in DCM (4 mL) was added 1 mL trifluoroacetic acid and the reaction solution was stirred at room temperature. After 12 h, the reaction solution was concentrated, the material re-suspended in DMSO, and the material purified by reverse-phase HPLC to provide 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-1-methyl-1H-indazol-7-yl)amino)-2-oxoethyl)piperidin-4-yl)propoxy)-2-methylphenyl)picolinic acid. (21 mg, 22 μmol, 46%) as a white solid. MS (ESI) m/z: 960.5 [M+H]⁺. ¹H NMR is consistent with structure.

### Example 225. Preparation of Compound 223

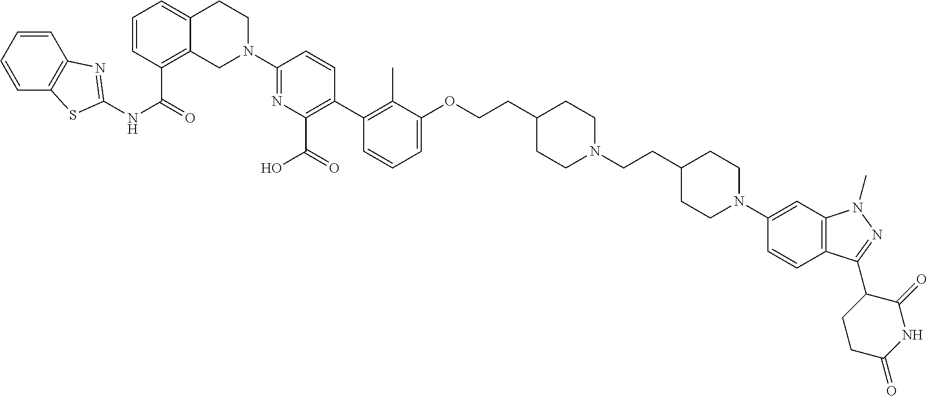 [SMILES: Cc1c(OCCC2CCN(CCC3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(2-(1-(2-(1-(3-(2,6-dioxopiperidin-3-yl)-1-methyl-1H-indazol-6-yl)piperidin-4-yl)ethyl)piperidin-4-yl)ethoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(2-(1-(2-(1-(3-(2,6-dioxopiperidin-3-yl)-1-methyl-1H-indazol-6-yl)piperidin-4-yl)ethyl)piperidin-4-yl)ethoxy)-2-methylphenyl)picolinate

To a solution of tert-butyl 6-(8-(benzo[d]thiazol-2-ylcarbamoyl)-3,4-dihydroisoquinolin-2(1H)-yl)-3-(2-methyl-3-(2-(piperidin-4-yl)ethoxy)phenyl)picolinate (77 mg, 1 equiv., 0.11 mmol) in DCM (5 mL) was added 2-(1-(3-(2,6-dioxopiperidin-3-yl)-1-methyl-1H-indazol-6-yl)piperidin-4-yl)acetaldehyde (41 mg, 1 equiv., 0.11 mmol), followed by sodium cyanoborohydride (35 mg, 32 μL, 5 equiv., 0.55 mmol) and acetic acid (20 mg, 19 μL, 3 equiv., 0.33 mmol). The reaction solution was stirred at room temperature. After 14 h, the reaction solution was concentrated, re-suspended in DMSO, and purified by reverse-phase HPLC to provide tert-butyl 6-(8-(benzo[d]thiazol-2-ylcarbamoyl)-3,4-dihydroisoquinolin-2(1H)-yl)-3-(3-(2-(1-(2-(1-(3-(2,6-dioxopiperidin-3-yl)-1-methyl-1H-indazol-6-yl)piperidin-4-yl)ethyl)piperidin-4-yl)ethoxy)-2-methylphenyl)picolinate (40 mg, 38 μmol, 34% yield) as an off-white solid. MS (ESI) m/z: 529.0 [M+H]⁺.

### Step B. Procedure for Preparation of 6-(8-(benzo[d]thiazol-2-ylcarbamoyl)-3,4-dihydroisoquinolin-2(1H)-yl)-3-(3-(2-(1-(2-(1-(3-(2,6-dioxopiperidin-3-yl)-1-methyl-1H-indazol-6-yl)piperidin-4-yl)ethyl)piperidin-4-yl)ethoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(2-(1-(2-(1-(3-(2,6-dioxopiperidin-3-yl)-1-methyl-1H-indazol-6-yl)piperidin-4-yl)ethyl)piperidin-4-yl)ethoxy)-2-methylphenyl)picolinate (10 mg, 1 equiv., 9.5 μmol) in DCM (4 mL) was added trifluoroacetic acid (1.1 mg, 0.73 μL, 1 equiv., 9.5 μmol) and the reaction solution was stirred at room temperature. After 16 h, the reaction solution was concentrated, re-suspended in DMSO, and purified by reverse-phase HPLC to provide 6-(8-(benzo[d]thiazol-2-ylcarbamoyl)-3,4-dihydroisoquinolin-2(1H)-yl)-3-(3-(2-(1-(2-(1-(3-(2,6-dioxopiperidin-3-yl)-1-methyl-1H-indazol-6-yl)piperidin-4-yl)ethyl)piperidin-4-yl)ethoxy)-2-methylphenyl)picolinic acid (6 mg, 6 μmol, 60% yield). MS (ESI) m/z: 501.3 [2M+H]*; ¹H NMR (400 MHz, DMSO) δ 10.76 (s, 1H), 7.94 (d, J 8.1 Hz, 1H), 7.70 (d, J 8.1 Hz, 1H), 7.56 (d, J 7.4 Hz, 1H), 7.33 (ddd, J=35.0, 14.7, 7.8 Hz, 7H), 7.00 (t, J=7.8 Hz, 1H), 6.82 (d, J=7.8 Hz, 3H), 6.74 (s, 1H), 6.58 (d, J=7.6 Hz, 1H), 6.47 (s, 1H), 4.91 (s, 2H), 4.17 (dd, J=9.2, 5.1 Hz, 2H), 3.92 (s, 2H), 3.82 (d, J=12.4 Hz, 5H), 3.68 (d, J=12.2 Hz, 2H), 2.95 (s, 2H), 2.81 (d, J=10.8 Hz, 3H), 2.64 (d, J=11.9 Hz, 3H), 2.09 (s, 1H), 1.83 (d, J=5.1 Hz, 5H), 1.69 (d, J=12.8 Hz, 3H), 1.61 (s, 3H), 1.41 (s, 1H), 1.33 (d, J=16.1 Hz, 3H), 1.17 (s, 3H).

### Example 226. Preparation of Compound 221

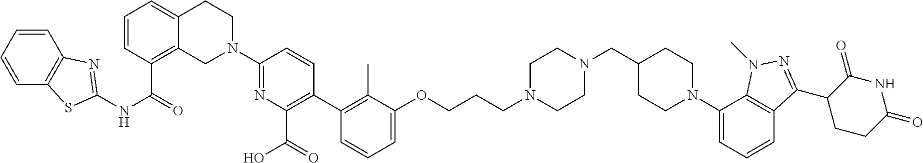 [SMILES: Cc1c(OCCCN2CCN(CC3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-(4-((1-(3-(2,6-dioxopiperidin-3-yl)-1-methyl-1H-indazol-7-yl)piperidin-4-yl)methyl)piperazin-1-yl)propoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(3-(4-((1-(3-(2,6-dioxopiperidin-3-yl)-1-methyl-1H-indazol-7-yl)piperidin-4-yl)methyl)piperazin-1-yl)propoxy)-2-methylphenyl)picolinate

To a solution of tert-butyl 6-(8-(benzo[d]thiazol-2-ylcarbamoyl)-3,4-dihydroisoquinolin-2(1H)-yl)-3-(3-(3-chloropropoxy)-2-methylphenyl)picolinate (100 mg, 1 equiv., 149 μmol) in NMP (1 mL) was added 3-(1-methyl-7-(4-(piperazin-1-ylmethyl)piperidin-1-yl)-1H-indazol-3-yl)piperidine-2,6-dione (63.4 mg, 1 equiv., 149 μmol), followed by sodium iodide (26.9 mg, 1.2 equiv., 179 μmol) and diisopropylethylamine (96.6 mg, 130 μL, 5 equiv., 747 μmol) and the reaction solution was stirred at 60° C. After 48 h, the reaction solution was concentrated, re-suspended in DMSO, and purified by reverse-phase HPLC to provide tert-butyl 6-(8-(benzo[d]thiazol-2-ylcarbamoyl)-3,4-dihydroisoquinolin-2(1H)-yl)-3-(3-(3-(4-((1-(3-(2,6-dioxopiperidin-3-yl)-1-methyl-1H-indazol-7-yl)piperidin-4-yl)methyl)piperazin-1-yl)propoxy)-2-methylphenyl)picolinate (45 mg, 43 μmol, 28% yield) as a white solid. MS (ESI) m/z: 529.5 [2M+H]⁺.

### Step B. Procedure for Preparation of 6-(8-(benzo[d]thiazol-2-ylcarbamoyl)-3,4-dihydroisoquinolin-2(1H)-yl)-3-(3-(3-(4-((1-(3-(2,6-dioxopiperidin-3-yl)-1-methyl-1H-indazol-7-yl)piperidin-4-yl)methyl)piperazin-1-yl)propoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(3-(4-((1-(3-(2,6-dioxopiperidin-3-yl)-1-methyl-1H-indazol-7-yl)piperidin-4-yl)methyl)piperazin-1-yl)propoxy)-2-methylphenyl)picolinate (45 mg, 1 equiv, 43 μmol) in DCM (2.4 mL) was added 0.6 mL TFA and the reaction solution was stirred at room temperature. After 40 h, the reaction solution was concentrated, re-suspended in water/acetonitrile, and lyophilized to provide 6-(8-(benzo[d]thiazol-2-ylcarbamoyl)-3,4-dihydroisoquinolin-2(1H)-yl)-3-(3-(3-(4-((1-(3-(2,6-dioxopiperidin-3-yl)-1-methyl-1H-indazol-7-yl)piperidin-4-yl)methyl)piperazin-1-yl)propoxy)-2-methylphenyl)picolinic acid trifluoroacetate (55 mg, 45 μmol, 110%) as an off-white solid. MS (ESI) m/z: 501.5 [M+H]⁺;

### Example 227. Preparation of Compound 228

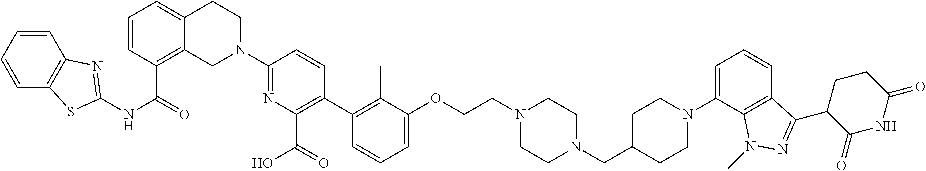 [SMILES: Cc1c(OCCN2CCN(CC3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(2-(4-((1-(3-(2,6-dioxopiperidin-3-yl)-1-methyl-1H-indazol-7-yl)piperidin-4-yl)methyl)piperazin-1-yl)ethoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(2-(4-((1-(3-(2,6-dioxopiperidin-3-yl)-1-methyl-1H-indazol-7-yl)piperidin-4-yl)methyl)piperazin-1-yl)ethoxy)-2-methylphenyl)picolinate

tert-Butyl 6-(8-(benzo[d]thiazol-2-ylcarbamoyl)-3,4-dihydroisoquinolin-2(1H)-yl)-3-(3-(2-chloroethoxy)-2-methylphenyl)picolinate (100 mg, 1 equiv., 153 μmol) was suspended in NMP (1 mL). To the mixture was added 3-(1-methyl-7-(4-(piperazin-1-ylmethyl)piperidin-1-yl)-1H-indazol-3-yl)piperidine-2,6-dione (64.8 mg, 1 equiv., 153 μmol), followed by sodium iodide (27.5 mg, 1.2 equiv, 183 μmol) and DIEA (98.6 mg, 133 μL, 5 equiv., 763 μmol). The mixture was then purged with nitrogen and heated at 60° C. After 48 h, the reaction solution was concentrated, re-suspended in DMSO, and purified by reverse-phase HPLC to provide tert-butyl 6-(8-(benzo[d]thiazol-2-ylcarbamoyl)-3,4-dihydroisoquinolin-2(1H)-yl)-3-(3-(2-(4-((1-(3-(2,6-dioxopiperidin-3-yl)-1-methyl-1H-indazol-7-yl)piperidin-4-yl)methyl)piperazin-1-yl)ethoxy)-2-methylphenyl)picolinate (41 mg, 39 μmol, 26% yield). MS (ESI) m/z: 522.5 [2M+H]⁺.

### Step B. Procedure for Preparation of 6-(8-(benzo[d]thiazol-2-ylcarbamoyl)-3,4-dihydroisoquinolin-2(1H)-yl)-3-(3-(2-(4-((1-(3-(2,6-dioxopiperidin-3-yl)-1-methyl-1H-indazol-7-yl)piperidin-4-yl)methyl)piperazin-1-yl)ethoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(2-(4-((1-(3-(2,6-dioxopiperidin-3-yl)-1-methyl-1H-indazol-7-yl)piperidin-4-yl)methyl)piperazin-1-yl)ethoxy)-2-methylphenyl)picolinate (41 mg, 1 equiv., 39 μmol) in DCM (2.4 mL) was added 0.6 mL TFA and the reaction solution was stirred at room temperature. After 40 h, the reaction solution was concentrated to provide 6-(8-(benzo[d]thiazol-2-ylcarbamoyl)-3,4-dihydroisoquinolin-2(1H)-yl)-3-(3-(2-(4-((1-(3-(2,6-dioxopiperidin-3-yl)-1-methyl-1H-indazol-7-yl)piperidin-4-yl)methyl)piperazin-1-yl)ethoxy)-2-methylphenyl)picolinic acid, trifluoroacetate (46 mg, 38 μmol, 96% yield) as a light brown solid. MS (ESI) m/z: 987.6 [M+H]⁺. ¹H NMR is consistent with structure.

### Example 228. Preparation of Compound 260

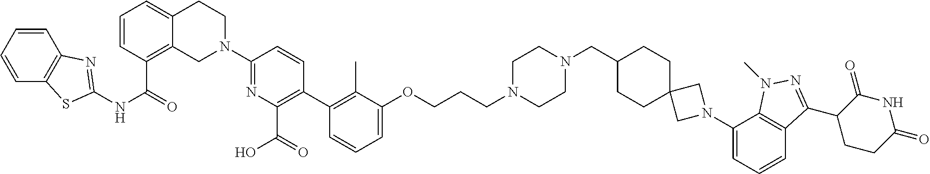 [SMILES: Cc1c(OCCCN2CCN(CC3CCC4(CC3)CN(c3cccc5c(C6CCC(=O)NC6=O)nn(C)c35)C4)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-(4-((2-(3-(2,6-dioxopiperidin-3-yl)-1-methyl-1H-indazol-7-yl)-2-azaspiro[3.5]nonan-7-yl)methyl)piperazin-1-yl)propoxy)-2-methylphenyl)picolinic acid

### Step A. Procedure for Preparation of (2-Azaspiro[3.5]nonan-7-yl)methanol

To a stirred solution of tert-butyl 7-(hydroxymethyl)-2-azaspiro[3.5]nonane-2-carboxylate (8.70 g, 34.07 mmol) in DCM (87.0 mL) was added 4M hydrochloric acid in dioxane (43.5 mL) at 0° C. and the resulting reaction mixture was stirred at room temperature. After 2 h, the reaction mixture was concentrated, and the crude solids were triturated with petroleum ether to afford (2-azaspiro[3.5]nonan-7-yl)methanol (6.07 g, 34.07 mmol), which was carried forward without further purification. ¹H NMR (DMSO-D6) δ 3.62-3.54 (m, 4H), 3.18 (d, J=6.00 Hz, 2H), 2.00-1.97 (m, 2H), 1.62-1.58 (m, 2H), 1.39-1.29 (m, 3H), 0.89-0.75 (m, 2H).

### Step B. Procedure for Preparation of (2-(3-(2,6-Bis(benzyloxy)pyridin-3-yl)-1-methyl-1H-indazol-7-yl)-2-azaspiro[3.5]nonan-7-yl)methanol

To a stirred solution of 3-(2,6-bis(benzyloxy)pyridin-3-yl)-7-bromo-1-methyl-1H-indazole (5.0 g, 9.99 mmol) and (2-azaspiro[3.5]nonan-7-yl)methanol (3.10 g, 19.98 mmol) in 1,4-dioxane (100 mL), was added cesium carbonate (9.76 g, 29.97 mmol) and the solution was degassed with argon for 20 minutes. Pd-PEPPSI-1HeptCl (0.49 g, 0.49 mmol) was added and the resulting reaction mixture was stirred at 100° C. After 16 h, the reaction mixture was cooled to room temperature, filtered through celite and concentrated. The resulting crude material was purified by silica gel column chromatography (40% ethyl acetate in petroleum ether) to provide (2-(3-(2,6-bis(benzyloxy)pyridin-3-yl)-1-methyl-1H-indazol-7-yl)-2-azaspiro[3.5]nonan-7-yl)methanol (4.05 g, 71 mmol, 71%) as a pale yellow solid. MS (ESI) m/z: 575.70 [M+H]⁺; ¹H NMR; 400 MHz, DMSO-d6: S 7.84 (d, J=8.00 Hz, 1H), 7.48-7.26 (m, 10H), 7.15 (d, J=8.00 Hz, 1H), 6.90 (t, J=7.60 Hz, 1H), 6.72 (d, J=7.20 Hz, 1H), 6.57 (d, J=8.00 Hz, 1H), 5.42 (d, J=6.00 Hz, 4H), 4.39 (t, J=5.20 Hz, 1H), 4.26 (s, 3H), 3.59 (d, J=20.00 Hz, 4H), 3.23 (t, J=5.60 Hz, 2H), 1.98 (d, J=13.20 Hz, 2H), 1.67 (d, J=10.80 Hz, 2H), 1.52-1.35 (m, 3H), 1.00 (q, J=11.60 Hz, 2H).

### Step C. Procedure for Preparation of (2-(3-(2,6-bis(benzyloxy)pyridin-3-yl)-1-methyl-1H-indazol-7-yl)-2-azaspiro[3.5]nonan-7-yl)methyl methanesulfonate

To a stirred solution of (2-(3-(2,6-bis(benzyloxy)pyridin-3-yl)-1-methyl-1H-indazol-7-yl)-2-azaspiro[3.5]nonan-7-yl)methanol (4.0 g, 6.95 mmol) in DCM (40 mL) was added triethylamine (2.9 mL, 20.87 mmol) followed by methanesulfonyl chloride (0.64 mL, 8.35 mmol) and the resulting reaction mixture was stirred at room temperature. After 1 h, the reaction solution was diluted with saturated aqueous sodium bicarbonate (20 mL) and extracted with dichloromethane (2×30 mL). The combined organic layers were dried over anhydrous sodium sulfate, filtered and concentrated to provide (2-(3-(2,6-bis(benzyloxy)pyridin-3-yl)-1-methyl-1H-indazol-7-yl)-2-azaspiro[3.5]nonan-7-yl)methyl methanesulfonate (4.31 g, 95% yield) as a pale yellow solid, which was carried forward without further purification.

MS (ESI) m/z: 653.6 [M+H]⁺.

### Step D. Procedure for Preparation of tert-Butyl 4-((2-(3-(2,6-bis(benzyloxy)pyridin-3-yl)-1-methyl-1H-indazol-7-yl)-2-azaspiro[3.5]nonan-7-yl)methyl)piperazine-1-carboxylate

To a stirred solution of (2-(3-(2,6-bis(benzyloxy)pyridin-3-yl)-1-methyl-1H-indazol-7-yl)-2-azaspiro[3.5]nonan-7-yl)methyl methanesulfonate (4.30 g, 6.58 mmol) and tert-butyl piperazine-1-carboxylate (4.90 g, 26.34 mmol) in DMSO (43.0 mL) was added N,N-diisopropylethylamine (3.44 mL, 19.76 mmol) and the reaction mixture was stirred at 100° C. After 16 h, the reaction solution was cooled to room temperature and diluted with cold water to precipitate the product out of solution. The resulting solids were collected by filtration and purified by silica gel column chromatography (30-40% EtOAc in petroleum ether) to provide tert-butyl 4-((2-(3-(2,6-bis(benzyloxy)pyridin-3-yl)-1-methyl-1H-indazol-7-yl)-2-azaspiro[3.5]nonan-7-yl)methyl)piperazine-1-carboxylate (2.84 g, 3.81 mmol, 58%) yield as an off-white solid. MS (ESI) m/z: 743.8 [M+H]⁺; ¹H NMR; 400 MHz, DMSO-d6: δ 7.84 (d, J=8.00 Hz, 1H), 7.47 (d, J=7.20 Hz, 2H), 7.41-7.26 (m, 8H), 7.15 (d, J=8.00 Hz, 1H), 6.90 (t, J=7.60 Hz, 1H), 6.71 (d, J=7.20 Hz, 1H), 6.57 (d, J=8.00 Hz, 1H), 5.42 (d, J=6.00 Hz, 4H), 4.25 (s, 3H), 3.58 (d, J=19.60 Hz, 4H), 3.32-3.30 (m, 3H), 2.27-2.26 (m, 4H), 2.09 (d, J=7.20 Hz, 2H), 1.96 (d, J=12.80 Hz, 2H), 1.70 (d, J=11.20 Hz, 2H), 1.49 (t, J=2.80 Hz, 3H), 1.39 (s, 9H), 1.00-0.21 (m, 2H).

### Step E. Procedure for Preparation of tert-butyl 4-((2-(3-(2,6-dioxopiperidin-3-yl)-1-methyl-1H-indazol-7-yl)-2-azaspiro[3.5]nonan-7-yl)methyl)piperazine-1-carboxylate

To a solution of tert-butyl 4-((2-(3-(2,6-bis(benzyloxy)pyridin-3-yl)-1-methyl-1H-indazol-7-yl)-2-azaspiro[3.5]nonan-7-yl)methyl)piperazine-1-carboxylate (2.50 g, 3.36 mmol) in THE (75.0 mL) was added palladium hydroxide on carbon (2.50 g, 100% w/w) and the reaction mixture was stirred under hydrogen atmosphere (60 psi) at room temperature. After 4 h, the reaction mixture was diluted with THE (60 mL) and filtered through celite and washed with 200 mL of THF: DCM (1:1). The combined organic solutions were concentrated to provide tert-butyl 4-((2-(3-(2,6-dioxopiperidin-3-yl)-1-methyl-1H-indazol-7-yl)-2-azaspiro[3.5]nonan-7-yl)methyl)piperazine-1-carboxylate (1.85 g, 3.26 mmol, 97% yield) as a pale yellow solid. MS (ESI) m/z: 565.4 [M+H]⁺.

### Step F. Procedure for Preparation of 3-(1-methyl-7-(7-(piperazin-1-ylmethyl)-2-azaspiro[3.5]nonan-2-yl)-1H-indazol-3-yl)piperidine-2,6-dione trifluoroacetate

To a stirred solution of tert-butyl 4-((2-(3-(2,6-dioxopiperidin-3-yl)-1-methyl-1H-indazol-7-yl)-2-azaspiro[3.5]nonan-7-yl)methyl)piperazine-1-carboxylate (1.80 g, 3.18 mmol) in DCM (18 mL) was added trifluoroacetic acid (18.0 mL) at 0° C., and the reaction mixture was stirred at room temperature. After 3 h, the reaction solution was concentrated, and the resulting crude material was triturated with diethyl ether to provide 3-(1-methyl-7-(7-(piperazin-1-ylmethyl)-2-azaspiro[3.5]nonan-2-yl)-1H-indazol-3-yl)piperidine-2,6-dione trifluoroacetate (2.75 g) as an off-white solid which was carried forward without further purification. MS (ESI) m/z: 465.5 [M+H]*; ¹H NMR; 400 MHz, DMSO-d6: δ 10.85 (s, 1H), 7.21 (d, J=7.60 Hz, 1H), 6.99 (t, J=7.60 Hz, 1H), 6.73 (d, J=7.20 Hz, 1H), 4.32-4.30 (m, 1H), 4.17 (s, 3H), 3.71-3.56 (br, 4H), 3.41-2.81 (br, 10H), 2.67-2.60 (m, 2H), 2.33-2.17 (m, 2H), 1.99 (d, J=12.80 Hz, 2H), 1.74-1.71 (m, 3H), 1.51 (t, J=12.80 Hz, 2H), 1.36-1.06 (m, 2H).

### Step G. Procedure for Preparation of tert-butyl 6-(8-(benzo[d]thiazol-2-ylcarbamoyl)-3,4-dihydroisoquinolin-2(1H)-yl)-3-(3-(3-(4-((2-(3-(2,6-dioxopiperidin-3-yl)-1-methyl-1H-indazol-7-yl)-2-azaspiro[3.5]nonan-7-yl)methyl)piperazin-1-yl)propoxy)-2-methylphenyl)picolinate

tert-butyl 6-(8-(benzo[d]thiazol-2-ylcarbamoyl)-3,4-dihydroisoquinolin-2(1H)-yl)-3-(3-(3-chloropropoxy)-2-methylphenyl)picolinate (60 mg, 90 μmol, 1 equiv.), 3-(1-methyl-7-(7-(piperazin-1-ylmethyl)-2-azaspiro[3.5]nonan-2-yl)-1H-indazol-3-yl)piperidine-2,6-dione (46 mg, 99 μmol, 1.1 equiv.) and sodium iodide (16 mg, 0.11 mmol, 1.2 equiv.) were suspended in NMP (2 mL). To the mixture was added diisopropylethylamine (58 mg, 78 μL, 0.45 mmol, 5 equiv.). The mixture was then purged with nitrogen and heated at 60° C. After 72 h, additional diisopropylethylamine (58 mg, 78 μL, 0.45 mmol, 5 equiv.) was added. The mixture was stirred at 60° C. and the crude material was then purified by reverse-phase HPLC to afford tert-butyl 6-(8-(benzo[d]thiazol-2-ylcarbamoyl)-3,4-dihydroisoquinolin-2(1H)-yl)-3-(3-(3-(4-((2-(3-(2,6-dioxopiperidin-3-yl)-1-methyl-1H-indazol-7-yl)-2-azaspiro[3.5]nonan-7-yl)methyl)piperazin-1-yl)propoxy)-2-methylphenyl)picolinate (19 mg, 17 μmol, 19% yield) as an off-white solid. MS (ESI) m/z: 549.6 [M+2H/2]*.

### Step H. Procedure for Preparation of 6-(8-(benzo[d]thiazol-2-ylcarbamoyl)-3,4-dihydroisoquinolin-2(1H)-yl)-3-(3-(3-(4-((2-(3-(2,6-dioxopiperidin-3-yl)-1-methyl-1H-indazol-7-yl)-2-azaspiro[3.5]nonan-7-yl)methyl)piperazin-1-yl)propoxy)-2-methylphenyl)picolinic acid

tert-butyl 6-(8-(benzo[d]thiazol-2-ylcarbamoyl)-3,4-dihydroisoquinolin-2(1H)-yl)-3-(3-(3-(4-((2-(3-(2,6-dioxopiperidin-3-yl)-1-methyl-1H-indazol-7-yl)-2-azaspiro[3.5]nonan-7-yl)methyl)piperazin-1-yl)propoxy)-2-methylphenyl)picolinate (19 mg, 1 equiv., 17 μmol) was dissolved in 1,2-dichloromethane (2.4 mL). To the mixture was added 0.6 mL trifluoroacetic acid and the mixture was stirred at room temperature. After 48 h, the reaction solution was concentrated, the material was resuspended in water/acetonitrile, and lyophilized to afford 6-(8-(benzo[d]thiazol-2-ylcarbamoyl)-3,4-dihydroisoquinolin-2(1H)-yl)-3-(3-(3-(4-((2-(3-(2,6-dioxopiperidin-3-yl)-1-methyl-1H-indazol-7-yl)-2-azaspiro[3.5]nonan-7-yl)methyl)piperazin-1-yl)propoxy)-2-methylphenyl)picolinic acid (22 mg, 17 μmol) as an off-white solid. MS (ESI) m/z: 1041.8 [M+H]⁺. ¹H NMR is consistent with structure.

### Example 229. Preparation of Compound 292

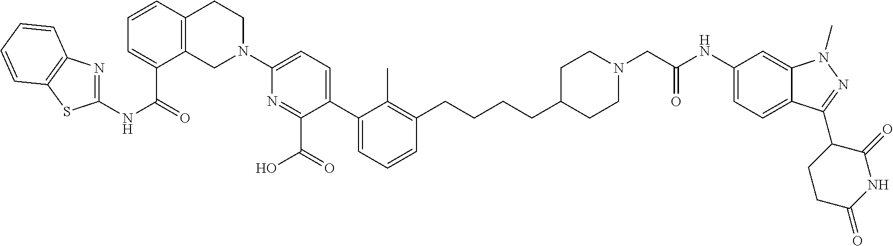 [SMILES: Cc1c(CCCCC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[4-[1-[2-[[3-(2,6-dioxo-3-piperidyl)-1-methyl-indazol-6-yl]amino]-2-oxo-ethyl]-4-piperidyl]butyl]-2-methyl-phenyl]pyridine-2-carboxylic acid

### Step A. Procedure for Preparation of tert-butyl 4-(3-oxopropyl)piperidine-1-carboxylate

To a solution of tert-butyl 4-(3-hydroxypropyl)piperidine-1-carboxylate (5.0 g, 20.55 mmol, 1.0 equiv.) in DCM (50 mL) was added DMP (10.46 g, 24.66 mmol, 7.64 mL, 1.2 equiv.) under 0° C. Then the mixture was stirred at 25° C. for 16 hours. The reaction mixture was then filtered and washed with ethyl acetate (30 mL×2). The filtrate was concentrated under reduced pressure to give a residue which was purified by flash silica gel chromatography (17˜35% ethyl acetate in petroleum ether) to give tert-butyl 4-(3-oxopropyl)piperidine-1-carboxylate (5.5 g, 19.67 mmol, 95.7% yield) as a light yellow oil.

¹H NMR (400 MHz, DMSO-d₆) δ=9.67 (t, J=1.6 Hz, 1H), 3.91 (m, 2H), 2.75-2.54 (m, 2H), 2.45 (m, 2H), 1.60 (d, J=12.8 Hz, 2H), 1.49-1.40 (m, 2H), 1.38 (s, 9H), 1.36-1.30 (m, 1H), 1.00-0.88 (m, 2H).

### Step B. Procedure for Preparation of tert-butyl 4-but-3-ynylpiperidine-1-carboxylate

A mixture of tert-butyl 4-(3-oxopropyl)piperidine-1-carboxylate (5.0 g, 20.72 mmol, 1.0 equiv.), 1-diazo-1-dimethoxyphosphoryl-propan-2-one (4.78 g, 24.86 mmol, 1.2 equiv.) and K₂CO₃ (7.16 g, 51.80 mmol, 2.5 equiv.) in MeOH (100 mL) was degassed and purged with N₂ three times. The reaction mixture was stirred at 40° C. for 16 hours under N₂ atmosphere. The reaction mixture was then concentrated under reduced pressure to give a residue which was purified by column chromatography (SiO₂, petroleum ether:ethyl acetate=20:1) to give tert-butyl 4-but-3-ynylpiperidine-1-carboxylate (4.38 g, 18.4 mmol, 89.0% yield) as a colorless oil.

¹H NMR (400 MHz, DMSO-d₆) δ=3.91 (d, J=11.6 Hz, 2H), 2.75 (t, J=2.4 Hz, 1H), 2.72-2.58 (m, 2H), 2.18 (m, 2H), 1.62 (d, J=12.4 Hz, 2H), 1.50 (m, 1H), 1.43-1.34 (m, 11H), 0.94 (m, 2H).

### Step C. Procedure for Preparation of tert-butyl 4-[4-(3-bromo-2-methyl-phenyl)but-3-ynyl]piperidine-1-carboxylate

A mixture of tert-butyl 4-but-3-ynylpiperidine-1-carboxylate (600 mg, 2.53 mmol, 1.0 equiv.), 1-bromo-3-iodo-2-methyl-benzene (900.79 mg, 3.03 mmol, 1.2 equiv.), TEA (1.28 g, 12.64 mmol, 1.76 mL, 5.0 equiv.), CuI (96.29 mg, 505.61 μmol, 0.2 equiv.), and Pd(PPh₃)₂Cl₂ (177.44 mg, 252.81 μmol, 0.1 equiv.) in DMF (20 mL) was degassed and purged with N₂. The reaction mixture was stirred at 60° C. for 16 hours under N₂ atmosphere. The reaction mixture was then diluted with water (50 mL) and extracted with ethyl acetate (40 mL×3). The combined organic layers were washed with brine (50 mL×2), dried over Na₂SO₄, filtered, and concentrated under reduced pressure to give a residue which was purified by column chromatography (SiO₂, petroleum ether: ethyl acetate=20:1) to give tert-butyl 4-[4-(3-bromo-2-methyl-phenyl)but-3-ynyl]piperidine-1-carboxylate (950 mg, 2.34 mmol, 92.4% yield) as a yellow oil.

¹H NMR (400 MHz, DMSO-d₆) δ=7.56 (d, J=8.0 Hz, 1H), 7.37 (d, J=7.6 Hz, 1H), 7.10 (t, J=8.0 Hz, 1H), 3.93 (d, J=12.8 Hz, 2H), 2.70-2.65 (m, 1H), 2.46 (s, 4H), 1.68 (d, J=13.2 Hz, 2H), 1.55-1.49 (m, 2H), 1.39 (s, 9H), 1.06-0.95 (m, 2H), −0.09 (s, 3H).

### Step D. Procedure for Preparation of tert-butyl 4-[4-(3-bromo-2-methyl-phenyl)butyl]piperidine-1-carboxylate

A mixture of tert-butyl 4-[4-(3-bromo-2-methyl-phenyl)but-3-ynyl]piperidine-1-carboxylate (950 mg, 2.34 mmol, 1 equiv.) and Rh/Al₂O₃ (300 mg, 2.92 mmol, 1.25 equiv.) in EtOAc (10 mL) was degassed and purged with N₂ three times and with H₂ three times. The reaction mixture was stirred at 25° C. for 16 hours under H₂ (15 Psi) atmosphere. The mixture was filtered, and the filter cake was washed with ethyl acetate (40 mL). The filtrate was concentrated under reduced pressure to give tert-butyl 4-[4-(3-bromo-2-methyl-phenyl)butyl]piperidine-1-carboxylate (700 mg, 1.71 mmol, 72.6% yield) as a colorless oil.

¹H NMR (400 MHz, DMSO-d₆) δ=7.56 (d, J=8.0 Hz, 1H), 7.37 (d, J=7.6 Hz, 1H), 7.10 (t, J=7.6 Hz, 1H), 3.93 (m, 2H), 2.75-2.62 (m, 2H), 2.52-2.51 (m, 3H), 2.46 (s, 4H), 1.68 (m, 2H), 1.57 (m, 1H), 1.51 (q, J=7.2 Hz, 2H), 1.38 (s, 11H), 1.10-0.92 (m, 2H).

### Step E. Procedure for Preparation of 4-[4-(3-bromo-2-methyl-phenyl)butyl]piperidine

To a solution of tert-butyl 4-[4-(3-bromo-2-methyl-phenyl)butyl]piperidine-1-carboxylate (700 mg, 1.71 mmol, 1.0 equiv.) in DCM (2 mL) was added HCl/dioxane (10 mL). The mixture was stirred at 25° C. for 16 hours. The reaction mixture was concentrated under reduced pressure to remove HCl/dioxane and DCM and then concentrated under reduced pressure to give 4-[4-(3-bromo-2-methyl-phenyl)butyl]piperidine (590 mg, 1.70 mmol, 99.7% yield) as a white solid.

¹H NMR (400 MHz, DMSO-d₆) δ=7.42 (d, J=7.6 Hz, 1H), 7.15 (d, J=7.6 Hz, 1H), 7.06-6.98 (m, 1H), 3.19 (m, 2H), 2.78 (m, 2H), 2.67-2.60 (m, 2H), 2.33 (s, 3H), 1.74 (m, 2H), 1.51-1.44 (m, 3H), 1.35-1.21 (m, 7H).

### Step F. Procedure for Preparation of ethyl 2-[4-[4-(3-bromo-2-methyl-phenyl)butyl]-1-piperidyl]acetate

A mixture of 4-[4-(3-bromo-2-methyl-phenyl)butyl]piperidine (300 mg, 865.22 μmol, 1.0 equiv.), ethyl 2-bromoacetate (130.04 mg, 778.70 μmol, 86.18 μL, 0.90 equiv.), TEA (262.65 mg, 2.60 mmol, 361.28 μL, 3.0 equiv.), and KI (28.73 mg, 173.04 μmol, 0.2 equiv.) in MeCN (5 mL) was stirred at 25° C. for 1 hour. The reaction mixture was diluted with water (40 mL) and extracted with ethyl acetate (40 mL×3). The combined organic layers were washed with brine (40 mL×2), dried over Na₂SO₄, filtered, and concentrated under reduced pressure to give ethyl 2-[4-[4-(3-bromo-2-methyl-phenyl)butyl]-1-piperidyl]acetate (280 mg, 706.4 μmol, 81.6% yield) as a yellow oil.

MS (ESI) m/z: 396.2 [M+H]⁺.

### Step G. Procedure for Preparation of tert-butyl 6-[8-(1,3-benzothiazol-2-ylcarbamoyl)-3,4-dihydro-1H-isoquinolin-2-yl]-3-[3-[4-[1-(2-ethoxy-2-oxo-ethyl)-4-piperidyl]butyl]-2-methyl-phenyl]pyridine-2-carboxylate

A mixture of ethyl 2-[4-[4-(3-bromo-2-methyl-phenyl)butyl]-1-piperidyl]acetate (280 mg, 706.43 μmol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (519.26 mg, 847.71 μmol, 1.2 equiv.), Ad₂nBuP Pd G₃ (102.89 mg, 141.29 μmol, 0.2 equiv.), and K₂CO₃ (146.45 mg, 1.06 mmol, 1.5 equiv.) in dioxane (5 mL) and H₂O (1 mL) was degassed and purged with N₂ three times. The reaction mixture was stirred at 100° C. for 1 hour under microwave. The reaction mixture was diluted with water (40 mL) and extracted with ethyl acetate (40 mL×3). The combined organic layers were washed with brine (40 mL×2), dried over Na₂SO₄, filtered, and concentrated under reduced pressure to give a residue which was purified by flash silica gel chromatography (30-40% ethyl acetate in petroleum ether) to give tert-butyl 6-[8-(1,3-benzothiazol-2-ylcarbamoyl)-3,4-dihydro-1H-isoquinolin-2-yl]-3-[3-[4-[1-(2-ethoxy-2-oxo-ethyl)-4-piperidyl]butyl]-2-methyl-phenyl]pyridine-2-carboxylate (270 mg, 267.63 μmol, 37.9% yield) as a yellow solid.

MS (ESI) m/z: 802.6 [M+H]⁺

### Step H. Procedure for Preparation of 2-[4-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenyl]butyl]-1-piperidyl]acetic acid

A mixture of tert-butyl 6-[8-(1,3-benzothiazol-2-ylcarbamoyl)-3,4-dihydro-1H-isoquinolin-2-yl]-3-[3-[4-[1-(2-ethoxy-2-oxo-ethyl)-4-piperidyl]butyl]-2-methyl-phenyl]pyridine-2-carboxylate (270 mg, 336.64 μmol, 1.0 equiv.) and LiOH·H₂O (42.38 mg, 1.01 mmol, 3.0 equiv.) in THE (2 mL) and H₂O (2 mL) was stirred at 25° C. for 12 hours under N₂ atmosphere. Afterwards, H₂O (5 mL) was added, and the pH of the reaction mixture was adjusted to ˜6 by progressively adding diluted HCl. The mixture was filtered to give 2-[4-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenyl]butyl]-1-piperidyl]acetic acid (240 mg, 216.44 μmol, 64.2% yield) as a yellow solid

MS (ESI) m/z: 775.0 [M+H]⁺

### Step I. Procedure for preparation of tert-butyl 6-[8-(1,3-benzothiazol-2-ylcarbamoyl)-3,4-dihydro-1H-isoquinolin-2-yl]-3-[3-[4-[1-[2-[[3-(2,6-dioxo-3-piperidyl)-1-methyl-indazol-6-yl]amino]-2-oxo-ethyl]-4-piperidyl]butyl]-2-methyl-phenyl]pyridine-2-carboxylate

A mixture of 2-[4-[4-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenyl]butyl]-1-piperidyl]acetic acid (100 mg, 129.20 μmol, 1.0 equiv.), 3-(6-amino-1-methyl-indazol-3-yl)piperidine-2,6-dione (45.70 mg, 155.04 μmol, 1.2 equiv., HCl), and EDCI (37.15 mg, 193.80 μmol, 1.5 equiv.) in pyridine (2 mL), was stirred at 25° C. for 16 hours. The reaction mixture was then concentrated under reduced pressure, diluted with water, and extracted with DCM (40 mL×3). The combined organic layers were washed with brine (40 mL×2), dried over Na₂SO₄, filtered, and concentrated under reduced pressure to give a residue which was purified by prep-TLC (SiO₂, petroleum ether/ethyl acetate=0:1) to give tert-butyl 6-[8-(1,3-benzothiazol-2-ylcarbamoyl)-3,4-dihydro-1H-isoquinolin-2-yl]-3-[3-[4-[1-[2-[[3-(2,6-dioxo-3-piperidyl)-1-methyl-indazol-6-yl]amino]-2-oxo-ethyl]-4-piperidyl]butyl]-2-methyl-phenyl]pyridine-2-carboxylate (70 mg, 65.68 μmol, 50.8% yield) as a red oil.

MS (ESI) m/z: 1014.6 [M+H]⁺.

### Step J. Procedure for Preparation of 6-[8-(1,3-benzothiazol-2-ylcarbamoyl)-3,4-dihydro-1H-isoquinolin-2-yl]-3-[3-[4-[1-[2-[[3-(2,6-dioxo-3-piperidyl)-1-methyl-indazol-6-yl]amino]-2-oxo-ethyl]-4-piperidyl]butyl]-2-methyl-phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[3-[4-[1-[2-[[3-(2,6-dioxo-3-piperidyl)-1-methyl-indazol-6-yl]amino]-2-oxo-ethyl]-4-piperidyl]butyl]-2-methyl-phenyl]pyridine-2-carboxylate (70 mg, 69.02 μmol, 1.0 equiv.) and DCM (1.5 mL) and TFA (0.5 mL) was stirred at 25° C. for 12 hours. The reaction mixture was concentrated under reduced pressure to give a residue which was purified by prep-HPLC to give 6-[8-(1,3-benzothiazol-2-ylcarbamoyl)-3,4-dihydro-1H-isoquinolin-2-yl]-3-[3-[4-[1-[2-[[3-(2,6-dioxo-3-piperidyl)-1-methyl-indazol-6-yl]amino]-2-oxo-ethyl]-4-piperidyl]butyl]-2-methyl-phenyl]pyridine-2-carboxylic acid (28.14 mg, 28.91 μmol, 41.9% yield) as a yellow solid.

MS (ESI) m/z: 958.5 [M+H]⁺.

¹H NMR (400 MHz, DMSO-d₆) δ=13.01-12.75 (m, 1H), 10.89 (s, 1H), 8.13 (s, 1H), 8.07-7.98 (m, 2H), 7.79 (d, J=8.0 Hz, 1H), 7.64 (m, 2H), 7.50-7.41 (m, 3H), 7.36 (m, 2H), 7.16 (m, 1H), 7.07 (q, J=7.6 Hz, 2H), 6.97 (d, J=8.8 Hz, 1H), 6.86 (m, 1H), 4.98 (m, 2H), 4.33 (m, 1H), 4.04-3.81 (m, 6H), 3.03 (m, 2H), 2.70-2.56 (m, 6H), 2.54 (s, 2H), 2.39-2.30 (m, 2H), 2.21-2.14 (m, 1H), 1.99 (s, 3H), 1.75 (m, 2H), 1.52 (m, 2H), 1.45-1.24 (m, 7H).

### Example 230: Preparation of Compound 293a

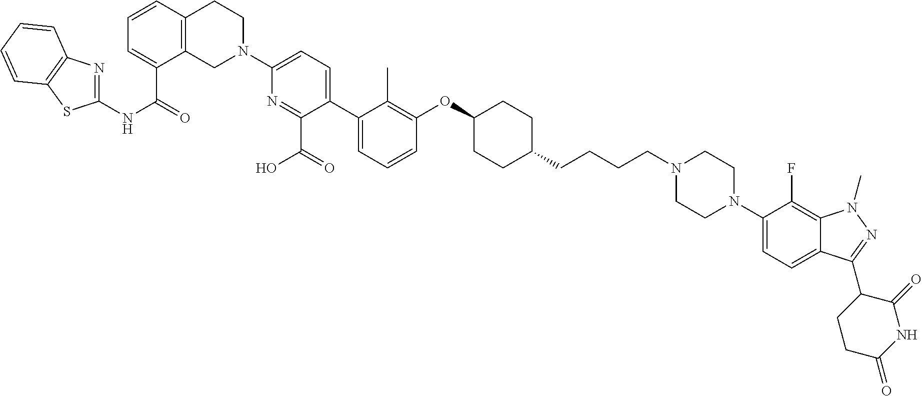 [SMILES: Cc1c(O[C@H]2CC[C@H](CCCCN3CCN(c4ccc5c(C6CCC(=O)NC6=O)nn(C)c5c4F)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-7-fluoro-1-methyl-1H-indazol-6-yl)piperazin-1-yl)butyl)cyclohexyl)oxy)-2-methylphenyl)picolinic acid

### Step A. Procedure for Preparation of (E)-4-bromo-2,3-difluorobenzaldehyde O-methyl oxime

To a solution of O-methylhydroxylamine (6.24 g, 74.66 mmol, 1.1 equiv., HCl) in DME (150 mL) was added K₂CO₃ (28.14 g, 203.62 mmol, 3.0 equiv.) and 4-bromo-2,3-difluoro-benzaldehyde (15.0 g, 67.87 mmol, 1.0 equiv.). The mixture was stirred at 40° C. for 3 hours. The reaction mixture was filtered and concentrated under reduced pressure to give (E)-4-bromo-2,3-difluorobenzaldehyde O-methyl oxime (52 g, crude) as a white solid.

### Step B. Procedure for Preparation of 6-bromo-7-fluoro-1H-indazole

To a solution of (E)-4-bromo-2,3-difluorobenzaldehyde O-methyl oxime (26 g, 103.98 mmol, 1.0 equiv.) in DME (260 mL) was added N₂H₄·H₂O (19.42 g, 380.17 mmol, 18.82 mL, 98% purity, 3.66 equiv.). The mixture was stirred at 90° C. for 16 hours. The reaction mixture was cooled to 20° C., concentrated under reduced pressure, added into H₂O, filtered, and concentrated under reduced pressure to give a residue. The residue was triturated with petroleum ether at 20° C. for 10 min to give 6-bromo-7-fluoro-1H-indazole (44.0 g, 193.70 mmol, 93.1% yield) as a white solid.

MS (ESI) m/z: 215.2 [M+H]⁺

¹H NMR (400 MHz, DMSO-d₆) δ=8.22 (d, J=3.4 Hz, 1H), 7.56 (d, J=8.5 Hz, 1H), 7.29 (dd, J=5.8, 8.5 Hz, 1H)

¹⁹F NMR (377 MHz, DMSO-d₆) δ=−125.15 (s, 1F)

### Step C. Procedure for Preparation of 6-bromo-7-fluoro-3-iodo-1H-indazole

To a solution of 6-bromo-7-fluoro-1H-indazole (44 g, 204.63 mmol, 1.0 equiv.) in DMF (400 mL) was added KOH (22.96 g, 409.26 mmol, 2.0 equiv.) and 12 (83.10 g, 327.41 mmol, 65.95 mL, 1.6 equiv.). The mixture was stirred at 25° C. for 16 hours. The reaction mixture was quenched by addition of saturated Na₂SO₃ solution (20 mL) at 20° C., diluted with H₂O, filtered, and concentrated under reduced pressure to give a residue. The residue was triturated with H₂O at 20° C. for 10 min to give 6-bromo-7-fluoro-3-iodo-1H-indazole (49.0 g, 134.96 mmol, 65.9% yield) as a light brown solid.

MS (ESI) m/z: 341.0 [M+H]⁺

¹H NMR (400 MHz, DMSO-d₆) δ=7.40-7.34 (m, 1H), 7.25-7.20 (m, 1H)

¹⁹F NMR (377 MHz, DMSO-d₆) δ=−124.27 (s, 1F)

### Step D. Procedure for Preparation of 6-bromo-7-fluoro-3-iodo-1-methyl-indazole

To a solution of 6-bromo-7-fluoro-3-iodo-1H-indazole (49.0 g, 143.73 mmol, 1.0 equiv.) in DMF (500 mL) was added K₂CO₃ (59.59 g, 431.19 mmol, 3.0 equiv.) and Mel (40.80 g, 287.46 mmol, 17.90 mL, 2.0 equiv.). The mixture was stirred at 40° C. for 16 hours. The reaction mixture was quenched by the addition of saturated NH₄Cl solution (100 mL) at 0° C., diluted with H₂O, and extracted with EtOAc (1000 mL×3). The combined organic layers were washed with H₂O, dried over Na₂SO₄, filtered, and concentrated under reduced pressure to give a residue. The residue was purified by column chromatography (SiO₂, petroleum ether/ethyl acetate=200/1 to 100/1) to give 6-bromo-7-fluoro-3-iodo-1-methyl-indazole (29.3 g, 81.75 mmol, 56.8% yield) as a white solid.

MS (ESI) m/z: 357.0 [M+H]⁺

¹H NMR (400 MHz, DMSO-d₆) δ=7.42-7.34 (m, 1H), 7.21 (d, J=8.8 Hz, 1H), 4.21-4.13 (m, 3H)

¹⁹F NMR (377 MHz, DMSO-d₆) δ=−126.90 (s, 1F)

### Step E. Procedure for Preparation of 3-(2,6-bis(benzyloxy)pyridin-3-yl)-6-bromo-7-fluoro-1-methyl-1H-indazole

A mixture of 6-bromo-7-fluoro-3-iodo-1-methyl-indazole (29.3 g, 82.55 mmol, 1.0 equiv.), 2,6-dibenzyloxy-3-(4,4,5,5-tetramethyl-1,3,2-dioxaborolan-2-yl)pyridine (37.89 g, 90.80 mmol, 1.1 equiv.), Cs₂CO₃ (80.69 g, 247.64 mmol, 3.0 equiv.), and Pd(dppf)Cl₂·CH₂Cl₂ (3.37 g, 4.13 mmol, 0.05 equiv.) in THE (300 mL) and H₂O (30 mL) was degassed and purged with N₂ three times. The mixture was stirred at 60° C. for 16 hours under N₂ atmosphere. The mixture was filtered and concentrated under reduced pressure to give a residue. The residue was purified by column chromatography (SiO₂, petroleum ether/ethyl acetate=200/1 to 50/1) to give 3-(2,6-bis(benzyloxy)pyridin-3-yl)-6-bromo-7-fluoro-1-methyl-1H-indazole (30.35 g, 53.95 mmol, 65.3% yield) as a light yellow solid.

MS (ESI) m/z: 520.2 [M+H]⁺

¹H NMR (400 MHz, CDCl₃) δ=7.82 (d, J=8.0 Hz, 1H), 7.42-7.38 (m, 2H), 7.36-7.29 (m, 3H), 7.27-7.19 (m, 6H), 6.99 (dd, J=5.6, 8.4 Hz, 1H), 6.48 (d, J=8.0 Hz, 1H), 5.40 (s, 2H), 5.37 (s, 2H), 4.23-4.18 (m, 3H)

¹⁹F NMR (377 MHz, CDCl₃) δ=−128.97 (s, 1F)

### Step F. Procedure for Preparation of tert-butyl 4-(3-(2,6-bis(benzyloxy)pyridin-3-yl)-7-fluoro-1-methyl-1H-indazol-6-yl)piperazine-1-carboxylate

A mixture of 3-(2,6-bis(benzyloxy)pyridin-3-yl)-6-bromo-7-fluoro-1-methyl-1H-indazole (500 mg, 964.55 μmol, 1 equiv.), tert-butyl piperazine-1-carboxylate (359.30 mg, 1.93 mmol, 2 equiv.), RuPhos (90.02 mg, 192.91 μmol, 0.2 equiv.), Pd₂(dba)₃ (88.33 mg, 96.46 μmol, 0.1 equiv.), and Cs₂CO₃ (942.81 mg, 2.89 mmol, 3 equiv.) in toluene (6 mL) was degassed and purged with N₂ three times, and then the mixture was stirred at 110° C. for 12 hours under N₂ atmosphere. The reaction mixture was filtered and concentrated under reduced pressure to give a residue. The residue was purified by flash silica gel chromatography (0˜20% ethyl acetate/petroleum ether) to give tert-butyl 4-(3-(2,6-bis(benzyloxy)pyridin-3-yl)-7-fluoro-1-methyl-1H-indazol-6-yl)piperazine-1-carboxylate (420 mg, 637.49 μmol, 66.0% yield) as a yellow oil.

MS (ESI) m/z: 624.3 [M+H]⁺.

¹H NMR (400 MHz, DMSO-d₆, EC11342-39-P1B1) δ=7.87 (d, J=8.0 Hz, 1H), 7.51-7.42 (m, 2H), 7.41-7.25 (m, 9H), 6.84 (dd, J=7.6, 8.4 Hz, 1H), 6.58 (d, J=8.0 Hz, 1H), 5.43 (d, J=12.8 Hz, 4H), 4.14 (s, 3H), 3.49 (d, J=4.4 Hz, 4H), 3.08-2.97 (m, 4H), 1.43 (s, 9H).

### Step G. Procedure for Preparation of tert-butyl 4-(3-(2,6-dioxopiperidin-3-yl)-7-fluoro-1-methyl-1H-indazol-6-yl)piperazine-1-carboxylate

To a solution of tert-butyl 4-(3-(2,6-bis(benzyloxy)pyridin-3-yl)-7-fluoro-1-methyl-1H-indazol-6-yl)piperazine-1-carboxylate (400 mg, 641.32 μmol, 1 equiv.) in EtOH (2 mL) and THF (2 mL) was added Pd/C (90.06 mg, 641.32 μmol, 1 equiv.) and Pd(OH)₂ (90.06 mg, 641.32 μmol, 1 equiv.) under N₂ atmosphere. The suspension was degassed under vacuum and purged with H₂ several times. The mixture was then stirred at 25° C. for 2 hours under H₂ (15 psi) atmosphere. The reaction mixture was filtered and concentrated under reduced pressure to give a residue. The residue was purified by flash silica gel chromatography (0-50% ethyl acetate/petroleum ether) to give tert-butyl 4-(3-(2,6-dioxopiperidin-3-yl)-7-fluoro-1-methyl-1H-indazol-6-yl)piperazine-1-carboxylate (180 mg, 402.92 μmol, 62.8% yield) as a white solid.

MS (ESI) m/z: 446.2 [M+H]⁺

### Step H. Procedure for Preparation of 3-(7-fluoro-1-methyl-6-(piperazin-1-yl)-1H-indazol-3-yl)piperidine-2,6-dione

A solution of tert-butyl 4-(3-(2,6-dioxopiperidin-3-yl)-7-fluoro-1-methyl-1H-indazol-6-yl)piperazine-1-carboxylate (190 mg, 426.50 μmol, 1 equiv.) in HCl/dioxane (2 mL) was stirred at 25° C. for 12 hours. The reaction mixture was filtered and concentrated under reduced pressure to give 3-(7-fluoro-1-methyl-6-(piperazin-1-yl)-1H-indazol-3-yl)piperidine-2,6-dione (150 mg, crude) as a white solid and used into the next step without further purification.

MS (ESI) m/z: 346.0 [M+H]⁺.

### Step I. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-7-fluoro-1-methyl-1H-indazol-6-yl)piperazin-1-yl)butyl)cyclohexyl)oxy)-2-methylphenyl)picolinate

A solution tert-butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate (90 mg, 120.82 μmol, 1 equiv.) and 3-(7-fluoro-1-methyl-6-(piperazin-1-yl)-1H-indazol-3-yl)piperidine-2,6-dione (55.36 mg, 144.98 μmol, 1.2 equiv., HCl) in DCM (1 mL) was stirred at 0° C. for 2 hours. Then NaBH(OAc)₃ (76.82 mg, 362.45 μmol, 3 equiv.) was added into the solution. The solution was stirred at 25° C. for 4 hours. The reaction mixture was filtered and concentrated under reduced pressure to give a residue. The residue was purified by prep-TLC to give tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-7-fluoro-1-methyl-1H-indazol-6-yl)piperazin-1-yl)butyl)cyclohexyl)oxy)-2-methylphenyl)picolinate (100 mg, 87.50 μmol, 72.4% yield) as a yellow solid.

MS (ESI) m/z: 1075.5 [M+H]⁺.

### Step J. Procedure for Preparation of 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-7-fluoro-1-methyl-1H-indazol-6-yl)piperazin-1-yl)butyl)cyclohexyl)oxy)-2-methylphenyl)picolinic acid

A solution of tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-7-fluoro-1-methyl-1H-indazol-6-yl)piperazin-1-yl)butyl)cyclohexyl)oxy)-2-methylphenyl)picolinate (90 mg, 83.77 μmol, 1 equiv.) in DCM (1 mL) and TFA (1 mL) was stirred at 25° C. for 12 hours. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-7-fluoro-1-methyl-1H-indazol-6-yl)piperazin-1-yl)butyl)cyclohexyl)oxy)-2-methylphenyl)picolinic acid (46.43 mg, 44.11 μmol, 52.6% yield) as a white solid.

MS (ESI) m/z: 1018.6 [M+H]⁺.

¹H NMR (400 MHz, DMSO-d₆) δ=10.89 (s, 1H), 8.14 (s, 1H), 8.03 (d, J=7.6 Hz, 1H), 7.79 (d, J=8.0 Hz, 1H), 7.62 (d, J=7.2 Hz, 1H), 7.41 (s, 7H), 7.10-7.03 (m, 1H), 6.93 (dd, J=9.2, 14.4 Hz, 3H), 6.61 (d, J=7.2 Hz, 1H), 4.97 (s, 2H), 4.32 (dd, J=5.2, 10.0 Hz, 1H), 4.22-4.13 (m, 1H), 4.06 (s, 3H), 3.91 (t, J=5.8 Hz, 2H), 3.09 (s, 4H), 3.02 (t, J=5.6 Hz, 2H), 2.68-2.59 (m, 2H), 2.54 (s, 2H), 2.37-2.31 (m, 4H), 2.19-2.09 (m, 1H), 2.07 (d, J=9.2 Hz, 2H), 1.87 (s, 3H), 1.81-1.74 (m, 2H), 1.48-1.40 (m, 2H), 1.37-1.28 (m, 4H), 1.23 (s, 3H), 1.11-0.99 (m, 2H).

### Example 231. Preparation of Compound 294

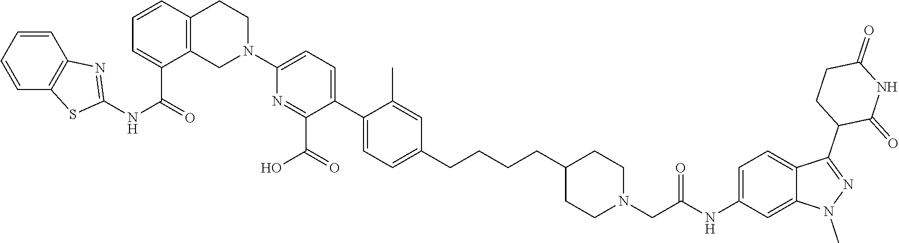 [SMILES: Cc1cc(CCCCC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O]

### 6-[8-(1,3-benzothiazol-2-ylcarbamoyl-3,4-dihydro-1H-isoquinolin-2-yl]-3-[4-[4-[1-[2-[[3-(2,6-dioxo-3-piperidyl)-1-methyl-indazol-6-yl]amino]-2-oxo-ethyl]-4-piperidyl]butyl]-2-methyl-phenyl]pyridine-2-carboxylic acid

### Step A. Procedure for Preparation of tert-butyl 4-[4-(4-bromo-3-methyl-phenyl)but-3-ynyl]piperidine-1-carboxylate

A mixture of 1-bromo-4-iodo-2-methyl-benzene (1.05 g, 3.54 mmol, 1.2 equiv.), tert-butyl 4-but-3-ynylpiperidine-1-carboxylate (700 mg, 2.95 mmol, 1.0 equiv.), TEA (1.49 g, 14.75 mmol, 2.05 mL, 5.0 equiv.), CuI (112.34 mg, 589.88 μmol, 0.2 equiv.), and dichloropalladiumtriphenylphosphane (207.02 mg, 294.94 μmol, 0.1 equiv.) in DMF (20 mL) was degassed and purged with N₂ three times. The mixture was stirred at 60° C. for 12 hours under N₂ atmosphere. The reaction mixture was partitioned between ethyl acetate (80 mL) and water (50 mL). The organic phase was separated, washed with brine (30 mL), dried over Na₂SO₄, filtered, and concentrated under reduced pressure to give a residue. The residue was purified by flash silica gel chromatography (0˜7% ethyl acetate/petroleum ether) to give tert-butyl 4-[4-(4-bromo-3-methyl-phenyl)but-3-ynyl]piperidine-1-carboxylate (880 mg, 2.01 mmol, 68.2% yield) as a yellow oil.

MS (ESI) m/z: 350.0 [M+H]⁺.

### Step B. Procedure for Preparation of tert-butyl 4-[4-(4-bromo-3-methyl-phenyl)butyl]piperidine-1-carboxylate

A mixture of tert-butyl 4-[4-(4-bromo-3-methyl-phenyl)but-3-ynyl]piperidine-1-carboxylate (880 mg, 2.17 mmol, 1.0 equiv.) and Rh/Al₂O₃ (300.85 mg, 2.92 mmol, 1.35 equiv.) in EtOAc (10 mL) was degassed and purged with H₂ three times, and then the mixture was stirred at 25° C. for 16 hours under H₂ atmosphere. The mixture was filtered, and the filter cake was washed with ethyl acetate (40 mL). The filtrate was concentrated under reduced pressure to give tert-butyl 4-[4-(4-bromo-3-methyl-phenyl)butyl]piperidine-1-carboxylate (800 mg, crude) as a yellow oil.

MS (ESI) m/z: 354.2 [M−56+H]⁺.

### Step C. Procedure for Preparation of 4-[4-(4-bromo-3-methyl-phenyl)butyl]piperidine

A solution of tert-butyl 4-[4-(4-bromo-3-methyl-phenyl)butyl]piperidine-1-carboxylate (800 mg, 1.95 mmol, 1.0 equiv.) in HCl/dioxane (10 mL) was stirred at 25° C. for 12 hours. The reaction mixture was concentrated under reduced pressure to give 4-[4-(4-bromo-3-methyl-phenyl)butyl]piperidine (660 mg, crude, HCl) as a white solid.

MS (ESI) m/z: 310.2 [M+H]⁺

¹H NMR (400 MHz, CDCl₃) δ=7.34 (d, J=8.0 Hz, 1H), 6.95 (s, 1H), 6.77 (d, J=8.0 Hz, 1H), 3.39 (d, J=12.0 Hz, 2H), 2.77-2.68 (m, 2H), 2.45 (t, J=8.0 Hz, 2H), 2.29 (s, 3H), 2.03 (s, 1H), 1.79 (d, J=13.6 Hz, 3H), 1.56 (d, J=2.8 Hz, 1H), 1.40 (dd, J=6.0, 7.2 Hz, 2H), 1.25 (d, J=3.6 Hz, 5H)

### Step D. Procedure for Preparation of ethyl 2-[4-[4-(4-bromo-3-methyl-phenyl)butyl]-1-piperidyl]acetate

To a solution of 4-[4-(4-bromo-3-methyl-phenyl)butyl]piperidine (300 mg, 865.22 μmol, 1.0 equiv., HCl) and ethyl 2-bromoacetate (130.04 mg, 778.70 μmol, 86.18 μL, 0.9 equiv.) in MeCN (5 mL) was added TEA (262.65 mg, 2.60 mmol, 361.28 μL, 3.0 equiv.) and KI (143.63 mg, 865.22 μmol, 1.0 equiv.). The mixture was stirred at 40° C. for 2 hours. The reaction mixture was concentrated under reduced pressure to give ethyl 2-[4-[4-(4-bromo-3-methyl-phenyl)butyl]-1-piperidyl]acetate (280 mg, crude) as a yellow solid.

MS (ESI) m/z: 398.2 [M+H]⁺

### Step E. Procedure for Preparation of tert-butyl 6-[8-(1,3-benzothiazol-2-ylcarbamoyl)-3,4-dihydro-1H-isoquinolin-2-yl]-3-[4-[4-[1-(2-ethoxy-2-oxo-ethyl)-4-piperidyl]butyl]-2-methyl-phenyl]pyridine-2-carboxylate

To a solution of ethyl 2-[4-[4-(4-bromo-3-methyl-phenyl)butyl]-1-piperidyl]acetate (280 mg, 706.43 μmol, 1.0 equiv.) and tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (519.26 mg, 847.71 μmol, 1.2 equiv.) in dioxane (5 mL) and H₂O (0.5 mL) was added K₂CO₃ (292.90 mg, 2.12 mmol, 3.0 equiv.) and Ad₂nBuP Pd G₃ (102.89 mg, 141.29 μmol, 0.2 equiv.). The mixture was stirred at 100° C. for 2 hours. The reaction mixture was partitioned between DCM (20 mL) and water (10 mL). The organic phase was separated, washed with brine (10 mL), dried over Na₂SO₄, filtered, and concentrated under reduced pressure to give a residue. The residue was purified by flash silica gel chromatography (0-39% ethyl acetate/petroleum ether) to give tert-butyl 6-[8-(1,3-benzothiazol-2-ylcarbamoyl)-3,4-dihydro-1H-isoquinolin-2-yl]-3-[4-[4-[1-(2-ethoxy-2-oxo-ethyl)-4-piperidyl]butyl]-2-methyl-phenyl]pyridine-2-carboxylate (320 mg, 279.2 mol, 39.5% yield) as a white solid.

MS (ESI) m/z: 802.6 [M+H]⁺

### Step F. Procedure for Preparation of 2-[4-[4-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenyl]butyl]-1-piperidyl]acetic acid

To a solution of tert-butyl 6-[8-(1,3-benzothiazol-2-ylcarbamoyl)-3,4-dihydro-1H-isoquinolin-2-yl]-3-[4-[4-[1-(2-ethoxy-2-oxo-ethyl)-4-piperidyl]butyl]-2-methyl-phenyl]pyridine-2-carboxylate (320 mg, 398.99 μmol, 1.0 equiv.) in THE (5 mL) and H₂O (1 mL) was added LiOH·H₂O (83.71 mg, 1.99 mmol, 5.0 equiv.). The mixture was stirred at 25° C. for 12 hours. The reaction mixture was acidified to pH=3-4 with citric acid, filtered, and concentrated under reduced pressure to give 2-[4-[4-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenyl]butyl]-1-piperidyl]acetic acid (300 mg, crude) as a white solid.

MS (ESI) m/z: 774.6 [M+H]⁺

### Step G. Procedure for Preparation of tert-butyl 6-[8-(1,3-benzothiazol-2-ylcarbamoyl)-3,4-dihydro-1H-isoquinolin-2-yl]-3-[4-[4-[1-[2-[[3-(2,6-dioxo-3-piperidyl)-1-methyl-indazol-6-yl]amino]-2-oxo-ethyl]-4-piperidyl]butyl]-2-methyl-phenyl]pyridine-2-carboxylate

To a solution of 2-[4-[4-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenyl]butyl]-1-piperidyl]acetic acid (100 mg, 129.20 μmol, 1.0 equiv.) and 3-(6-amino-1-methyl-indazol-3-yl)piperidine-2,6-dione (33.37 mg, 129.20 μmol, 1.0 equiv.) in pyridine (1 mL) was added EDCI (37.15 mg, 193.80 μmol, 1.5 equiv.). The mixture was stirred at 25° C. for 12 hours. The reaction mixture was partitioned between ethyl acetate and water. The organic phase was separated, washed with brine, dried over Na₂SO₄, filtered, and concentrated under reduced pressure to give a residue. The residue was purified by prep-TLC (SiO₂, petroleum ether:ethyl acetate=0:1) to give tert-butyl 6-[8-(1,3-benzothiazol-2-ylcarbamoyl)-3,4-dihydro-1H-isoquinolin-2-yl]-3-[4-[4-[1-[2-[[3-(2,6-dioxo-3-piperidyl)-1-methyl-indazol-6-yl]amino]-2-oxo-ethyl]-4-piperidyl]butyl]-2-methyl-phenyl]pyridine-2-carboxylate (70 mg, 66.9 μmol, 51.8% yield) as a white solid.

MS (ESI) m/z: 1014.4 [M+H]⁺

### Step H. Procedure for Preparation of 6-[8-(1,3-benzothiazol-2-ylcarbamoyl)-3,4-dihydro-1H-isoquinolin-2-yl]-3-[4-[4-[1-[2-[[3-(2,6-dioxo-3-piperidyl)-1-methyl-indazol-6-yl]amino]-2-oxo-ethyl]-4-piperidyl]butyl]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[4-[4-[1-[2-[[3-(2,6-dioxo-3-piperidyl)-1-methyl-indazol-6-yl]amino]-2-oxo-ethyl]-4-piperidyl]butyl]-2-methyl-phenyl]pyridine-2-carboxylate (70 mg, 69.02 μmol, 1.0 equiv.) in DCM (1 mL) was added TFA (7.87 mg, 69.02 μmol, 5.13 μL, 1.0 equiv.). The mixture was stirred at 25° C. for 12 hours. The reaction mixture was concentrated under reduced pressure to give a residue. The residue was purified by reverse-phase HPLC to give 6-[8-(1,3-benzothiazol-2-ylcarbamoyl)-3,4-dihydro-1H-isoquinolin-2-yl]-3-[4-[4-[1-[2-[[3-(2,6-dioxo-3-piperidyl)-1-methyl-indazol-6-yl]amino]-2-oxo-ethyl]-4-piperidyl]butyl]-2-methyl-phenyl]pyridine-2-carboxylic acid (25.45 mg, 26.3 μmol, 38.1% yield) as a yellow solid.

MS (ESI) m/z: 958.6 [M+H]⁺

¹H NMR (400 MHz, DMSO-d6) δ=12.87 (s, 1H), 10.88 (s, 1H), 10.03-9.65 (m, 1H), 8.13 (s, 1H), 8.07-8.00 (m, 2H), 7.79 (d, J=7.6 Hz, 1H), 7.68-7.60 (m, 2H), 7.51-7.42 (m, 3H), 7.41-7.31 (m, 2H), 7.20 (s, 1H), 7.04 (s, 1H), 7.00-6.89 (m, 3H), 4.97 (s, 2H), 4.33 (dd, J=5.2, 9.6 Hz, 1H), 3.96-3.87 (m, 5H), 3.02 (t, J=5.6 Hz, 2H), 2.65-2.60 (m, 2H), 2.59-2.54 (m, 5H), 2.22-2.15 (m, 2H), 2.03 (s, 3H), 1.91-1.62 (m, 3H), 1.61-1.44 (m, 3H), 1.40-1.16 (m, 8H)

### Example 232. Preparation of Compound 295

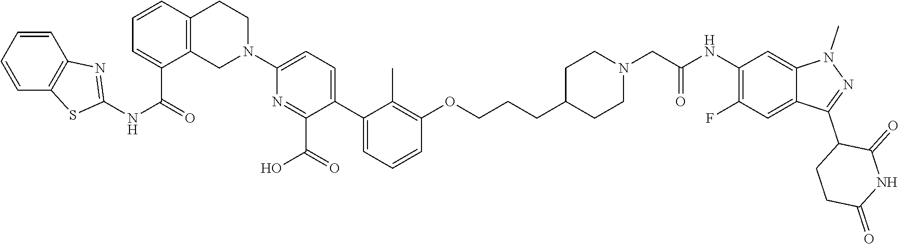 [SMILES: Cc1c(OCCCC2CCN(CC(=O)Nc3cc4c(cc3F)c(C3CCC(=O)NC3=O)nn4C)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-5-fluoro-1-methyl-indazol-6-yl]amino]-2-oxo-ethyl]-4-piperidyl]propoxy]-2-methyl-phenyl]pyridine-2-carboxylic acid

### Step A. Procedure for Preparation of tert-butyl 4-[3-(3-bromo-2-methyl-phenoxy)propyl]piperidine-1-carboxylate

A mixture of 3-bromo-2-methyl-phenol (600 mg, 3.21 mmol, 1 equiv.), tert-butyl 4-(3-hydroxypropyl)piperidine-1-carboxylate (936.76 mg, 3.85 mmol, 1.2 equiv.), and 2-(tributyl-phosphanylidene)acetonitrile (1.55 g, 6.42 mmol, 2 equiv.) in toluene (10 mL) was degassed and purged with N₂ three times. The mixture was stirred at 120° C. for 4 hours under N₂ atmosphere. The reaction mixture was filtered and concentrated under reduced pressure to give a residue. The residue was purified by flash silica gel chromatography (0˜11% ethyl acetate/petroleum ether) to give tert-butyl 4-[3-(3-bromo-2-methyl-phenoxy)propyl]piperidine-1-carboxylate (1.2 g, 2.91 mmol, 90.7% yield) as a white solid.

¹H NMR (400 MHz, DMSO-d₆) δ=7.17-7.13 (m, 1H), 7.12-7.07 (m, 1H), 6.96 (d, J=7.6 Hz, 1H), 4.01-3.86 (m, 4H), 2.75-2.58 (m, 2H), 2.23 (s, 3H), 1.80-1.71 (m, 2H), 1.67-1.64 (m, 2H), 1.49-1.42 (m, 1H), 1.39 (s, 9H), 1.37-1.33 (m, 2H), 1.04-0.89 (m, 2H).

### Step B. Procedure for Preparation of 4-[3-(3-bromo-2-methyl-phenoxy)propyl]piperidine

A solution of tert-butyl 4-[3-(3-bromo-2-methyl-phenoxy)propyl]piperidine-1-carboxylate (1.2 g, 2.91 mmol, 1 equiv.) in DCM (9 mL) and TFA (1 mL) was stirred at 25° C. for 1 hour. The reaction mixture was filtered and concentrated under reduced pressure to 4-[3-(3-bromo-2-methyl-phenoxy)propyl]piperidine (1.45 g, crude) as a white solid.

### Step C. Procedure for Preparation of ethyl 2-[4-[3-(3-bromo-2-methyl-phenoxy)propyl]-1-piperidyl]acetate

A mixture of 4-[3-(3-bromo-2-methyl-phenoxy)propyl]piperidine (1.3 g, 2.44 mmol, 1 equiv., TFA), ethyl 2-bromoacetate (366.70 mg, 2.20 mmol, 243.01 μL, 0.9 equiv.), and K₂CO₃ (1.01 g, 7.32 mmol, 3 equiv.) in MeCN (10 mL) was degassed and purged with N₂ three times. The mixture was stirred at 60° C. for 2 hours under N₂ atmosphere. The reaction mixture was filtered and concentrated under reduced pressure to give a residue. The residue was purified by flash silica gel chromatography (0˜25% ethyl acetate/petroleum ether) to give ethyl 2-[4-[3-(3-bromo-2-methyl-phenoxy)propyl]-1-piperidyl]acetate (700 mg, 1.76 mmol, 72.0% yield) as a white solid.

MS (ESI) m/z: 400.0 [M+H]⁺

### Step D. Procedure for Preparation of tert-butyl 6-[8-(1,3-benzothiazol-2-ylcarbamoyl)-3,4-dihydro-1H-isoquinolin-2-yl]-3-[3-[3-[1-(2-ethoxy-2-oxo-ethyl)-4-piperidyl]propoxy]-2-methyl-phenyl]pyridine-2-carboxylate

A mixture of ethyl 2-[4-[3-(3-bromo-2-methyl-phenoxy)propyl]-1-piperidyl]acetate (180 mg, 451.88 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (332.16 mg, 542.26 μmol, 1.2 equiv.), Ad₂nBuP Pd G₃ (98.73 mg, 135.56 μmol, 0.3 equiv.), and KF (39.38 mg, 677.82 μmol, 15.88 μL, 1.5 equiv.) in dioxane (4 mL) and H₂O (0.4 mL) was degassed and purged with N₂ three times. The mixture was stirred at 80° C. for 1 hour under microwave. The reaction mixture was filtered and concentrated under reduced pressure to give a residue. The residue was purified by flash silica gel chromatography (0˜24% ethyl acetate/petroleum ether) to give tert-butyl 6-[8-(1,3-benzothiazol-2-ylcarbamoyl)-3,4-dihydro-1H-isoquinolin-2-yl]-3-[3-[3-[1-(2-ethoxy-2-oxo-ethyl)-4-piperidyl]propoxy]-2-methyl-phenyl]pyridine-2-carboxylate (200 mg, 248.7 μmol, 55.0% yield) as a white solid.

MS (ESI) m/z: 804.7 [M+H]⁺

### Step E. Procedure for Preparation of 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-1-piperidyl]acetic acid

A mixture of tert-butyl 6-[8-(1,3-benzothiazol-2-ylcarbamoyl)-3,4-dihydro-1H-isoquinolin-2-yl]-3-[3-[3-[1-(2-ethoxy-2-oxo-ethyl)-4-piperidyl]propoxy]-2-methyl-phenyl]pyridine-2-carboxylate (200 mg, 248.75 μmol, 1 equiv.) and lithium hydroxide hydrate (31.32 mg, 746.26 μmol, 3 equiv.) in THE (2 mL) and H₂O (0.4 mL) was degassed and purged with N₂ three times. The mixture was stirred at 40° C. for 3 hours under N₂ atmosphere. The reaction mixture was concentrated under reduced pressure to remove THF. The resulting residue was diluted with H₂O and extracted with DCM/MeOH (10:1) (3 mL×3). The combined organic layers were washed with H₂O (3 mL×3), filtered, and concentrated under reduced pressure to give 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-1-piperidyl]acetic acid (150 mg, crude) as a white solid.

MS (ESI) m/z: 776.6 [M+H]⁺

### Step F. Procedure for Preparation of tert-butyl 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-5-fluoro-1-methyl-indazol-6-yl]amino]-2-oxo-ethyl]-4-piperidyl]propoxy]-2-methyl-phenyl]pyridine-2-carboxylate

A mixture of 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-1-piperidyl]acetic acid (80 mg, 103.10 μmol, 1 equiv.), 3-(6-amino-5-fluoro-1-methyl-indazol-3-yl)piperidine-2,6-dione (28.48 mg, 103.10 μmol, 1 equiv.), and EDCI (59.29 mg, 309.30 μmol, 3 equiv.) in pyridine (3 mL) was degassed and purged with N₂ three times. The mixture was stirred at 40° C. for 2 hours under N₂ atmosphere. The reaction mixture was filtered and concentrated under reduced pressure to give a residue. The residue was purified by flash silica gel chromatography (0-38% ethyl acetate/petroleum ether) to give tert-butyl 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-5-fluoro-1-methyl-indazol-6-yl]amino]-2-oxo-ethyl]-4-piperidyl]propoxy]-2-methyl-phenyl]pyridine-2-carboxylate (100 mg, 96.69 μmol, 93.7% yield) as a white solid.

MS (ESI) m/z: 1034.4 [M+H]⁺

### Step G. Procedure for Preparation of 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-5-fluoro-1-methyl-indazol-6-yl]amino]-2-oxo-ethyl]-4-piperidyl]propoxy]-2-methyl-phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-5-fluoro-1-methyl-indazol-6-yl]amino]-2-oxo-ethyl]-4-piperidyl]propoxy]-2-methyl-phenyl]pyridine-2-carboxylate (100 mg, 96.69 μmol, 1 equiv.) in TFA (1 mL) and DCM (1 mL) was stirred at 40° C. for 1 hour. The reaction mixture was filtered and concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-5-fluoro-1-methyl-indazol-6-yl]amino]-2-oxo-ethyl]-4-piperidyl]propoxy]-2-methyl-phenyl]pyridine-2-carboxylic acid (17.10 mg, 16.78 μmol, 17.3% yield) as an off-white solid.

MS (ESI) m/z: 978.8 [M+H]⁺

¹H NMR (400 MHz, DMSO-d₆) δ=12.86 (d, J=8.0 Hz, 1H), 12.69-12.41 (m, 1H), 10.89 (s, 1H), 8.32-8.22 (m, 1H), 8.03 (d, J=7.6 Hz, 1H), 7.79 (d, J=7.6 Hz, 1H), 7.70-7.59 (m, 2H), 7.52-7.28 (m, 6H), 7.13-7.06 (m, 1H), 6.97 (d, J=8.4 Hz, 1H), 6.88 (d, J=8.4 Hz, 1H), 6.63 (d, J=8.0 Hz, 1H), 5.08-4.86 (m, 2H), 4.38-4.29 (m, 1H), 3.96 (s, 3H), 3.95-3.90 (m, 2H), 3.22-3.15 (m, 2H), 3.04-3.01 (m, 2H), 2.94-2.85 (m, 1H), 2.68-2.60 (m, 4H), 2.34-2.30 (m, 4H), 2.20-2.13 (m, 2H), 1.90 (s, 3H), 1.82-1.73 (m, 4H), 1.43-1.40 (m, 2H), 1.28-1.19 (m, 2H).

### Example 233. Preparation of Compound 297a

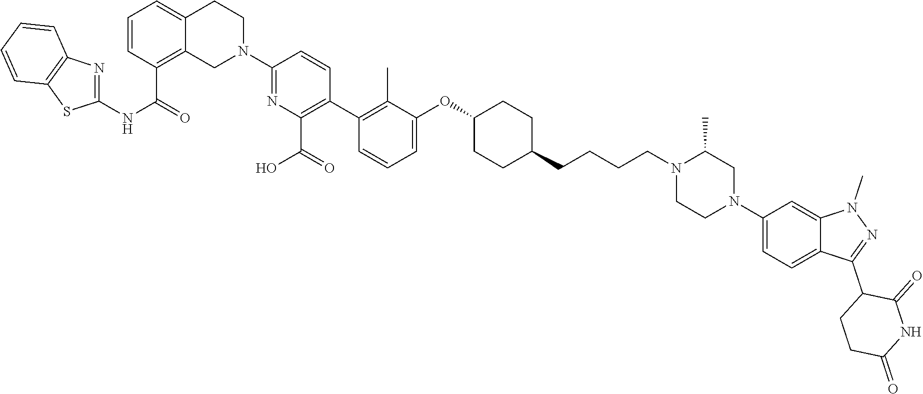 [SMILES: Cc1c(O[C@H]2CC[C@H](CCCCN3CCN(c4ccc5c(C6CCC(=O)NC6=O)nn(C)c5c4)C[C@H]3C)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(4-((2R)-4-(3-(2,6-dioxopiperidin-3-yl)-1-methyl-1H-indazol-6-yl)-2-methylpiperazin-1-yl)butyl)cyclohexyl)oxy)-2-methylphenyl)picolinic acid

### Step A. Procedure for Preparation of methyl (1r,4r)-4-(3-bromo-2-methylphenoxy)cyclohexane-1-carboxylate

A mixture of methyl (1s,4s)-4-hydroxycyclohexane-1-carboxylate (10.0 g, 63.21 mmol, 1.0 equiv.), 3-bromo-2-methyl-phenol (14.19 g, 75.86 mmol, 1.2 equiv.), and 2-(tributyl-λ5-phosphanylidene)acetonitrile (21.36 g, 88.50 mmol, 1.4 equiv.) in toluene (100 mL) was degassed and purged with N₂ three times, and then the mixture was stirred at 125° C. for 16 hours under N₂ atmosphere. The reaction mixture was concentrated under reduced pressure to give a residue. The residue was purified by flash silica gel chromatography (0-7% ethyl acetate/petroleum ether) to give methyl (1r,4r)-4-(3-bromo-2-methylphenoxy)cyclohexane-1-carboxylate (76 g, 139.36 mmol, crude) as a light yellow oil.

### Step B. Procedure for Preparation of ((1r,4r)-4-(3-bromo-2-methylphenoxy)cyclohexyl)methanol

To mixture of methyl (1r,4r)-4-(3-bromo-2-methylphenoxy)cyclohexane-1-carboxylate (25.0 g, 45.84 mmol, 60% purity, 1.0 equiv.) in THE (200 mL) was added LiAlH₄ (1.91 g, 50.43 mmol, 1.1 equiv.) at 0° C. for 10 min under N₂, and then the mixture was stirred at 0° C. for 2 hours under N₂ atmosphere. The mixture solution was quenched by Na₂SO₄·10H₂O (50 g), filtered, and concentrated under reduced pressure to give a residue. The residue was purified by column chromatography (SiO₂, petroleum ether/ethyl acetate=20/1 to 5/1) to give ((1r,4r)-4-(3-bromo-2-methylphenoxy)cyclohexyl)methanol (34.0 g, 113.20 mmol, 82.3% yield) as a colorless oil.

¹H NMR (400 MHz, DMSO-d₆) δ=7.13 (d, J=7.6 Hz, 1H), 7.06 (t, J=8.0 Hz, 1H), 7.01 (d, J=7.6 Hz, 1H), 4.44 (t, J=5.2 Hz, 1H), 4.25-4.18 (m, 1H), 3.23 (t, J=5.6 Hz, 2H), 2.21 (s, 3H), 2.06 (dd, J=2.8, 12.0 Hz, 2H), 1.78 (d, J=12.0 Hz, 2H), 1.39-1.30 (m, 3H), 1.09-1.02 (m, 2H)

### Step C. Procedure for Preparation of (1r,4r)-4-(3-bromo-2-methylphenoxy)cyclohexane-1-carbaldehyde

To a solution of oxalyl dichloride (9.33 g, 73.52 mmol, 6.44 mL, 2.0 equiv.) in DCM (80 mL) was added DMSO (11.49 g, 147.04 mmol, 11.49 mL, 4.0 equiv.) in DCM (40 mL) dropwise at −70° C. under N₂ for 30 min. The reaction mixture was stirred at −70° C. for 1 hour. ((1r,4r)-4-(3-bromo-2-methylphenoxy)cyclohexyl)methanol (11 g, 36.76 mmol, 1.0 equiv.) in DCM (40 mL) was added into the mixture at −70° C. under N₂ for 30 min. The resulting mixture was stirred at −70° C. for 1 hour under N₂. TEA (22.32 g, 220.56 mmol, 30.70 mL, 6.0 equiv.) in DCM (40 mL) was added into the mixture, and the reaction mixture was stirred at −70° C. for 1 hour. The reaction mixture was quenched by addition of H₂O, diluted, and extracted with DCM. The combined organic layers were washed with brine (400 mL×2), dried over Na₂SO₄, filtered, and concentrated under reduced pressure to give (1r,4r)-4-(3-bromo-2-methylphenoxy)cyclohexane-1-carbaldehyde (33.0 g, crude) as a yellow solid.

¹H NMR (400 MHz, DMSO-d₆) δ=9.60 (s, 1H), 7.14 (d, J=8.0 Hz, 1H), 7.07 (t, J=8.0 Hz, 1H), 7.02 (d, J=8.0 Hz, 1H), 4.32-4.27 (m, 1H), 2.38-2.33 (m, 1H), 2.22 (s, 3H), 2.02-1.93 (m, 4H), 1.54-1.38 (m, 4H)

### Step D. Procedure for Preparation of 1-(((1r,4r)-4-((E)-4-(benzyloxy)but-1-en-1-yl)cyclohexyl)oxy)-3-bromo-2-methylbenzene

To a mixture of 3-benzyloxypropyl(triphenyl)phosphonium; bromide (24.80 g, 50.47 mmol, 1.2 equiv.) in THE (150 mL) was added LiHMDS (1 M, 63.09 mL, 1.5 equiv.) dropwise at −70° C. under N₂ in THE (150 mL). The mixture was stirred at −50° C. for 3 hours, and then treated with (1r,4r)-4-(3-bromo-2-methylphenoxy)cyclohexane-1-carbaldehyde (12.5 g, 42.06 mmol, 1.0 equiv.). The mixture was stirred at 25° C. for 16 hours under N₂ atmosphere. The mixture was quenched by aqueous NH₄Cl (1000 mL) and extracted with EtOAc (1000 mL×2). The combined organic layers were washed with brine (500 mL×2) and then concentrated under reduced pressure to give a residue. The residue was purified by flash silica gel chromatography (0-5% ethyl acetate/petroleum ether) to give 1-(((1r,4r)-4-((E)-4-(benzyloxy)but-1-en-1-yl)cyclohexyl)oxy)-3-bromo-2-methylbenzene (24.0 g, 50.11 mmol, 59.5% yield) as a colorless oil.

¹H NMR (400 MHz, DMSO-d₆) δ=7.40-7.27 (m, 5H), 7.14 (d, J=8.0 Hz, 1H), 7.07 (t, J=8.0 Hz, 1H), 7.02 (d, J=8.0 Hz, 1H), 5.37-5.23 (m, 2H), 4.46 (s, 2H), 4.27-4.20 (m, 1H), 3.43 (t, J=6.8 Hz, 2H), 2.34-2.27 (m, 3H), 2.21 (s, 3H), 2.04-2.00 (m, 2H), 1.64-1.60 (m, 2H), 1.46-1.36 (m, 2H), 1.26-1.16 (m, 2H).

### Step E. Procedure for Preparation of 4-((1r,4s)-4-(3-bromo-2-methylphenoxy)cyclohexyl)butan-1-ol

To a solution of 1-(((1r,4r)-4-((E)-4-(benzyloxy)but-1-en-1-yl)cyclohexyl)oxy)-3-bromo-2-methylbenzene (16.0 g, 37.26 mmol, 1.0 equiv.) in ethyl acetate (50 mL) was added PtO₂ (5.08 g, 22.36 mmol, 0.6 equiv.) under N₂. The suspension was degassed under vacuum and purged with H₂ several times. The mixture was stirred under H₂ (50 psi) at 45° C. for 16 hours. The reaction mixture was filtered, and the filtrate was concentrated under reduced pressure to give a residue. The residue was purified by flash silica gel chromatography (0˜30% ethyl acetate/petroleum ether) to give 4-((1r,4s)-4-(3-bromo-2-methylphenoxy)cyclohexyl)butan-1-ol (3.6 g, 9.15 mmol, 24.5% yield) as a light brown oil.

¹H NMR (400 MHz, DMSO-d₆) δ=7.13 (d, J=8.0 Hz, 1H), 7.07 (t, J=8.0 Hz, 1H), 7.01 (d, J=8.0 Hz, 1H), 4.33-4.331 (m, 1H), 4.25-4.19 (m, 1H), 3.40-3.36 (m, 2H), 2.21 (s, 3H), 2.05-2.02 (m, 2H), 1.78-1.74 (m, 2H), 1.41-1.16 (m, 9H), 1.07-0.97 (m, 2H)

### Step F. Procedure for Preparation of 4-((1r,4s)-4-(3-bromo-2-methylphenoxy)cyclohexyl)butanal

To a solution of oxalyl dichloride (2.60 g, 20.51 mmol, 1.80 mL, 2.0 equiv.) in DCM (80 mL) was added DMSO (3.21 g, 41.02 mmol, 3.21 mL, 4.0 equiv.) in DCM (40 mL) dropwise at −70° C. under N₂ for 30 min. The reaction mixture was stirred at −70° C. for 1 hour. 4-((1r,4s)-4-(3-bromo-2-methylphenoxy)cyclohexyl)butan-1-ol (3.5 g, 10.26 mmol, 1.0 equiv.) in DCM (40 mL) was added into the mixture at −70° C. under N₂ for 30 min, and the resulting mixture was stirred at −70° C. for 1 hour under N₂. TEA (6.23 g, 61.53 mmol, 8.56 mL, 6.0 equiv.) in DCM (40 mL) was added into the mixture and stirred at −70° C. for 1 hour. The reaction mixture was quenched by addition of H₂O (400 mL), and extracted with DCM (300 mL×1). The combined organic layers were washed with brine (400 mL×2), dried over Na₂SO₄, filtered, and concentrated under reduced pressure to give 4-((1r,4s)-4-(3-bromo-2-methylphenoxy)cyclohexyl)butanal (3.4 g, 8.58 mmol, 83.7% yield) as a yellow oil.

¹H NMR (400 MHz, DMSO-d₆) δ=9.66 (s, 1H), 7.13 (d, J=8.0 Hz, 1H), 7.06 (t, J=8.0 Hz, 1H), 7.01 (d, J=8.0 Hz, 1H), 4.26-4.16 (m, 1H), 2.43-2.39 (m, 2H), 2.20 (s, 3H), 2.03 (d, J=10.0 Hz, 2H), 1.76 (d, J=12.0 Hz, 2H), 1.57-1.50 (m, 2H), 1.39-1.29 (m, 2H), 1.26-1.26 (m, 3H), 1.07-0.98 (m, 2H)

### Step G. Procedure for Preparation of tert-butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate

A mixture of 4-((1r,4s)-4-(3-bromo-2-methylphenoxy)cyclohexyl)butanal (3.3 g, 8.33 mmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (5.10 g, 8.33 mmol, 1.0 equiv.), Ad₂nBuP Pd G₃ (910.09 mg, 1.25 mmol, 0.15 equiv.), and K₂CO₃ (3.45 g, 24.99 mmol, 3.0 equiv.) in dioxane (10 mL) and H₂O (1 mL) was degassed and purged with N₂ three times. The mixture was stirred at 100° C. for 3 hours under N₂ atmosphere. The reaction mixture was diluted by addition of H₂O (300 mL) and extracted with ethyl acetate (500 mL×2). The combined organic layers were washed with brine (250 mL×2), dried over Na₂SO₄, filtered, and concentrated under reduced pressure to give a residue. The residue was purified by flash silica gel chromatography (0-30% ethyl acetate/petroleum ether) to give tert-butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate (2.1 g, 2.62 mmol, 31.4% yield) as a yellow solid.

MS (ESI) m/z: 745.1 [M+H]⁺.

### Step H. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(4-((2R)-4-(3-(2,6-dioxopiperidin-3-yl)-1-methyl-1H-indazol-6-yl)-2-methylpiperazin-1-yl)butyl)cyclohexyl)oxy)-2-methylphenyl)picolinate

To a solution of tert-butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate (150 mg, 201.36 mol, 1 equiv.) in DCM (2 mL) was added 3-[1-methyl-6-[(3R)-3-methylpiperazin-1-yl]indazol-3-yl]piperidine-2,6-dione (75.62 mg, 221.49 μmol, 1.1 equiv.). The mixture was stirred at 25° C. for 1 hour, and then NaBH(OAc)₃ (128.03 mg, 604.08 μmol, 3 equiv.) was added. The mixture was stirred at 25° C. for 16 hours. The reaction mixture was diluted with H₂O (20 mL) and extracted with DCM (20 mL×3). The combined organic layers were washed with brine (30 mL), dried over Na₂SO₄, filtered, and concentrated under reduced pressure to afford tert-butyl 6-(8-(benzo[d]thiazol-2-ylcarbamoyl)-3,4-dihydroisoquinolin-2(1H)-yl)-3-(3-(((1s,4r)-4-(4-((2R)-4-(3-(2,6-dioxopiperidin-3-yl)-1-methyl-1H-indazol-6-yl)-2-methylpiperazin-1-yl)butyl)cyclohexyl)oxy)-2-methylphenyl)picolinate (180 mg, crude) as a yellow oil.

### Step I. Procedure for preparation of 6-(8-(benzo[d]thiazol-2-ylcarbamoyl)-3,4-dihydroisoquinolin-2(1H)-yl)-3-(3-(((1s,4r)-4-(4-((2R)-4-(3-(2,6-dioxopiperidin-3-yl)-1-methyl-1H-indazol-6-yl)-2-methylpiperazin-1-yl)butyl)cyclohexyl)oxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(((1s,4r)-4-(4-((2R)-4-(3-(2,6-dioxopiperidin-3-yl)-1-methyl-1H-indazol-6-yl)-2-methylpiperazin-1-yl)butyl)cyclohexyl)oxy)-2-methylphenyl)picolinate (180 mg, 75.68 μmol, 1 equiv.) in DCM (2 mL) was added TFA (3.07 g, 26.92 mmol, 2 mL, 355.78 equiv.). The mixture was stirred at 25° C. for 16 hours. The reaction mixture was concentrated under reduced pressure to give a residue. The residue was purified by prep-HPLC to afford 6-(8-(benzo[d]thiazol-2-ylcarbamoyl)-3,4-dihydroisoquinolin-2(1H)-yl)-3-(3-(((1s,4r)-4-(4-((2R)-4-(3-(2,6-dioxopiperidin-3-yl)-1-methyl-1H-indazol-6-yl)-2-methylpiperazin-1-yl)butyl)cyclohexyl)oxy)-2-methylphenyl)picolinic acid (27.57 mg, 26.64 μmol, 35.2% yield) as a yellow solid.

MS (ESI) m/z: 1014.6 [M+H]⁺.

¹H NMR (400 MHz, DMSO-d₆) δ=10.85 (s, 1H), 8.03 (d, J=8.0 Hz, 1H), 7.79 (d, J=8.0 Hz, 1H), 7.62 (d, J=7.2 Hz, 1H), 7.50-7.42 (m, 4H), 7.39-7.33 (m, 2H), 7.08-7.04 (m, 1H), 6.95-6.90 (m, 3H), 6.82 (s, 1H), 6.62 (d, J=7.6 Hz, 1H), 4.97 (s, 2H), 4.27-4.24 (m, 1H), 4.20-4.15 (m, 1H), 3.92-3.89 (m, 5H), 3.53-3.51 (m, 2H), 3.02 (t, J=5.6 Hz, 2H), 2.94-2.84 (m, 2H), 2.74-2.67 (m, 2H), 2.62-2.59 (m, 2H), 2.39-2.13 (m, 5H), 2.08-2.06 (m, 2H), 1.87 (s, 3H), 1.79-1.77 (m, 2H), 1.43-1.24 (m, 9H), 1.09-1.03 (m, 5H)

### Example 234. Preparation of Compound 298a

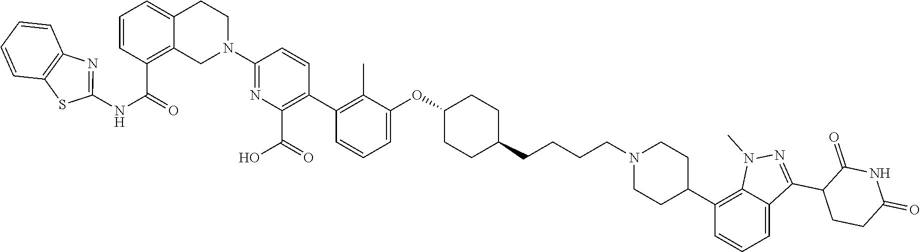 [SMILES: Cc1c(O[C@H]2CC[C@H](CCCCN3CCC(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-7-yl)piperidin-1-yl)butyl)cyclohexyl)oxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-7-yl)piperidin-1-yl)butyl)cyclohexyl)oxy)-2-methylphenyl)picolinate

To a solution of tert-butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate (150 mg, 201.36 mol, 1.0 equiv.) in DCM (2 mL) was added AcOH (12.09 mg, 201.36 μmol, 11.53 μL, 1.0 equiv.) and 3-(1-methyl-7-(piperidin-4-yl)-1H-indazol-3-yl)piperidine-2,6-dione (72.29 mg, 221.49 mol, 1.1 equiv.). The mixture was stirred at 25° C. for 1 hour. Then NaBH(OAc)₃ (128.03 mg, 604.08 μmol, 3.0 equiv.) was added into the mixture, and the resulting mixture was stirred at 25° C. for 11 hours. The reaction mixture was concentrated under reduced pressure to remove solvent. The residue was diluted with H₂O (50 mL) and extracted with DCM (50 mL×2). The combined organic layers were washed with brine (50 mL), dried over Na₂SO₄, filtered, and concentrated under reduced pressure to give a residue. The residue was used in the next step without further purification to give tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-7-yl)piperidin-1-yl)butyl)cyclohexyl)oxy)-2-methylphenyl)picolinate (100 mg, 94.76 μmol, 47.0% yield) as a yellow oil.

### Step B. Procedure for Preparation of 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-7-yl)piperidin-1-yl)butyl)cyclohexyl)oxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-7-yl)piperidin-1-yl)butyl)cyclohexyl)oxy)-2-methylphenyl)picolinate (100 mg, 94.76 μmol, 1.0 equiv.) in TFA (1 mL) was added DCM (1 mL). The mixture was stirred at 40° C. for 1 hour. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-7-yl)piperidin-1-yl)butyl)cyclohexyl)oxy)-2-methylphenyl)picolinic acid (31.22 mg, 29.96 μmol, 31.6% yield) as a white solid.

MS (ESI) m/z: 1000.0 [M+H]⁺.

¹H NMR (400 MHz, DMSO-d₆) δ=10.89 (s, 1H), 8.16 (s, 1H), 8.03 (d, J=8.0 Hz, 1H), 7.79 (d, J=8.0 Hz, 1H), 7.63 (d, J=7.2 Hz, 1H), 7.55 (d, J=8.0 Hz, 1H), 7.50-7.42 (m, 3H), 7.40-7.33 (m, 2H), 7.26 (d, J=7.2 Hz, 1H), 7.07 (t, J=7.6 Hz, 2H), 6.97-6.90 (m, 2H), 6.63 (d, J=7.2 Hz, 1H), 4.98 (s, 2H), 4.35 (dd, J=5.2, 9.8 Hz, 1H), 4.21 (s, 3H), 3.96-3.89 (m, 2H), 4.00 (s, 1H), 3.08 (dd, J=1.2, 9.4 Hz, 2H), 3.03 (t, J=5.2 Hz, 2H), 2.71-2.65 (m, 2H), 2.36-2.31 (m, 4H), 2.20-2.11 (m, 2H), 2.11-2.06 (m, 2H), 1.88 (s, 5H), 1.80 (d, J=12.0 Hz, 4H), 1.53-1.45 (m, 2H), 1.38-1.20 (m, 8H), 1.06 (d, J=11.6 Hz, 2H)

### Example 235. Preparation of Compound 299a

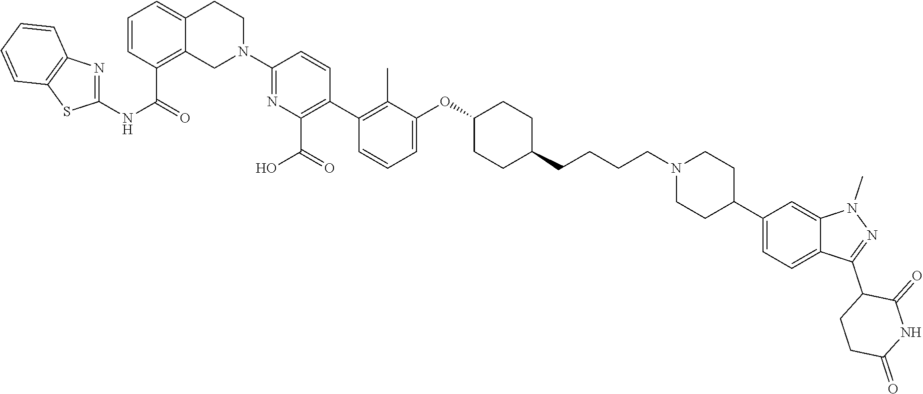 [SMILES: Cc1c(O[C@H]2CC[C@H](CCCCN3CCC(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperidin-1-yl)butyl)cyclohexyl)oxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperidin-1-yl)butyl)cyclohexyl)oxy)-2-methylphenyl)picolinate

To a solution of tert-butyl 6-(8-(benzo[d]thiazol-2-ylcarbamoyl)-3,4-dihydroisoquinolin-2(1H)-10 yl)-3-(2-methyl-3-(((1s,4r)-4-(4-oxobutyl)cyclohexyl)oxy)phenyl)picolinate (100 mg, 134.24 μmol, 1 equiv.) and 3-(1-methyl-6-(piperidin-4-yl)-1H-indazol-3-yl)piperidine-2,6-dione (52.58 mg, 161.09 μmol, 1.2 eq) in DCM (2 mL) was added NaBH(OAc)₃ (85.35 mg, 402.72 μmol, 3 equiv.). The mixture was stirred at 25° C. for 15 hours. The reaction mixture was concentrated under reduced pressure to give tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperidin-1-yl)butyl)cyclohexyl)oxy)-2-methylphenyl)picolinate (155 mg, 108.77 μmol, 81.0% yield) as a yellow solid.

MS (ESI) m/z: 528.8 [M/2+H]⁺.

### Step B. Procedure for Preparation of 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperidin-1-yl)butyl)cyclohexyl)oxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperidin-1-yl)butyl)cyclohexyl)oxy)-2-methylphenyl)picolinate (150 mg, 142.14 μmol, 1 equiv.) in DCM (1 mL) and TFA (1 mL) was stirred at 25° C. for 15 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperidin-1-yl)butyl)cyclohexyl)oxy)-2-methylphenyl)picolinic acid (35.5 mg, 34.0 μmol, 23.9% yield) as a yellow solid

MS (ESI) m/z: 999.8 [M+H]⁺.

¹H NMR (400 MHz, DMSO-d₆) δ=13.04-12.76 (m, 1H), 10.89 (s, 1H), 8.13 (s, 1H), 8.03 (d, J=8.0 Hz, 1H), 7.79 (d, J=8.0 Hz, 1H), 7.67 (d, J=8.4 Hz, 1H), 7.62 (d, J=7.2 Hz, 1H), 7.49-7.43 (m, 3H), 7.41-7.33 (m, 3H), 7.10-7.01 (m, 2H), 6.95 (dd, J=8.8, 16.8 Hz, 2H), 6.62 (d, J=7.6 Hz, 1H), 4.98 (s, 2H), 4.34 (dd, J=5.2, 10.0 Hz, 1H), 4.23-4.18 (m, 1H), 3.98 (s, 3H), 3.94-3.89 (m, 4H), 3.62 (d, J=12.0 Hz, 2H), 3.10 (d, J=8.8 Hz, 2H), 3.05-2.93 (m, 4H), 2.67 (dd, J=1.6, 3.6 Hz, 1H), 2.65-2.59 (m, 2H), 2.38-2.30 (m, 2H), 2.23-2.13 (m, 2H), 2.12-2.06 (m, 4H), 1.98-1.92 (m, 2H), 1.87 (s, 3H), 1.80 (d, J=11.6 Hz, 2H), 1.68 (s, 1H), 1.34 (d, J=6.8 Hz, 2H), 1.27 (d, J=5.2 Hz, 2H), 1.12-1.02 (m, 2H)

### Example 236. Preparation of Compound 300a

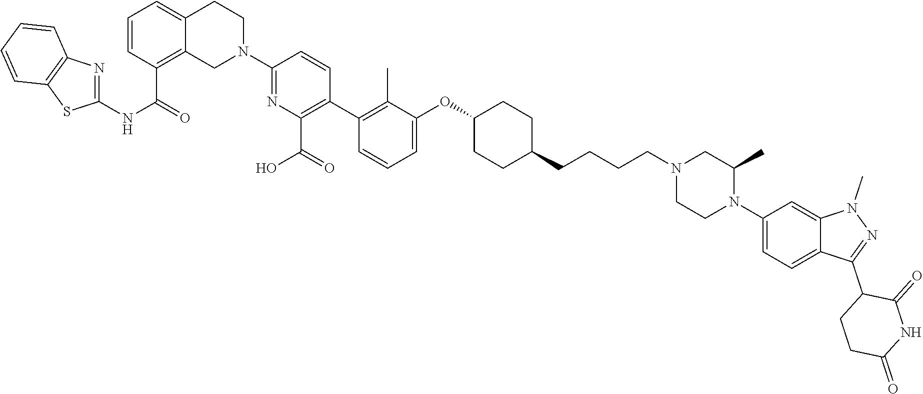 [SMILES: Cc1c(O[C@H]2CC[C@H](CCCCN3CCN(c4ccc5c(C6CCC(=O)NC6=O)nn(C)c5c4)[C@H](C)C3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(4-((3R)-4-(3-(2,6-dioxopiperidin-3-yl)-1-methyl-1H-indazol-6-yl)-3-methylpiperazin-1-yl)butyl)cyclohexyl)oxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(4-((3R)-4-(3-(2,6-dioxopiperidin-3-yl)-1-methyl-1H-indazol-6-yl)-3-methylpiperazin-1-yl)butyl)cyclohexyl)oxy)-2-methylphenyl)picolinate

To a solution of tert-butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate (100 mg, 134.24 mol, 1 equiv.) and 3-[1-methyl-6-[(2R)-2-methylpiperazin-1-yl]indazol-3-yl]piperidine-2,6-dione (45.83 mg, 134.24 μmol, 1 equiv.) in DCM (2 mL) was added NaBH(OAc)₃ (227.61 mg, 1.07 mmol, 8 equiv.). The mixture was stirred at 25° C. for 16 hours. The reaction mixture was concentrated under reduced pressure to give a residue. The residue was purified by prep-TLC (SiO₂, DCM:MeOH=12:1) to give tert-butyl 6-(8-(benzo[d]thiazol-2-ylcarbamoyl)-3,4-dihydroisoquinolin-2(1H)-yl)-3-(3-(((1s,4r)-4-(4-((3R)-4-(3-(2,6-dioxopiperidin-3-yl)-1-methyl-1H-indazol-6-yl)-3-methylpiperazin-1-yl)butyl)cyclohexyl)oxy)-2-methylphenyl)picolinate (55 mg, 49.84 μmol, 37.1% yield) as a yellow oil.

MS (ESI) m/z: 1071.2 [M+H]⁺.

### Step B. Procedure for Preparation of 6-(8-(benzo[d]thiazol-2-ylcarbamoyl)-3,4-dihydroisoquinolin-2(1H)-yl)-3-(3-(((1s,4r)-4-(4-((3R)-4-(3-(2,6-dioxopiperidin-3-yl)-1-methyl-1H-indazol-6-yl)-3-methylpiperazin-1-yl)butyl)cyclohexyl)oxy)-2-methylphenyl)picolinic acid

A solution of tert-butyl 6-(8-(benzo[d]thiazol-2-ylcarbamoyl)-3,4-dihydroisoquinolin-2(1H)-yl)-3-(3-(((1s,4r)-4-(4-((3R)-4-(3-(2,6-dioxopiperidin-3-yl)-1-methyl-1H-indazol-6-yl)-3-methylpiperazin-1-yl)butyl)cyclohexyl)oxy)-2-methylphenyl)picolinate (55 mg, 51.39 μmol, 1 equiv.) in DCM (1 mL) and TFA (1 mL) was stirred at 25° C. for 16 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-(4-((3R)-4-(3-(2,6-dioxopiperidin-3-yl)-1-methyl-1H-indazol-6-yl)-3-methylpiperazin-1-yl)butyl)cyclohexyl)oxy)-2-methylphenyl)picolinic acid (19.08 mg, 18.36 μmol, 35.72% yield, 97.58% purity) as a white solid.

¹H NMR (400 MHz, DMSO-d₆) δ=10.85 (s, 1H), 8.15 (s, 1H), 8.03 (d, J=8.0 Hz, 1H), 7.79 (d, J=8.8 Hz, 1H), 7.62 (d, J=7.6 Hz, 1H), 7.52-7.42 (m, 4H), 7.40-7.30 (m, 2H), 7.06 (d, J=8.0 Hz, 1H), 7.00-6.86 (m, 3H), 6.78 (s, 1H), 6.61 (d, J=7.6 Hz, 1H), 4.98 (s, 2H), 4.27-4.22 (m, 1H), 4.15-4.08 (m, 1H), 3.94-3.90 (m, 2H), 3.88 (s, 3H), 3.06-3.00 (m, 3H), 2.88 (d, J=4.0 Hz, 1H), 2.64-2.60 (m, 2H), 2.28 (s, 2H), 2.20-2.12 (m, 3H), 2.08 (s, 3H), 1.93 (s, 1H), 1.87 (s, 3H), 1.82-1.76 (m, 2H), 1.49-1.22 (m, 11H), 1.02 (d, J=5.6 Hz, 5H)

### Example 237: Preparation of Compound 300b

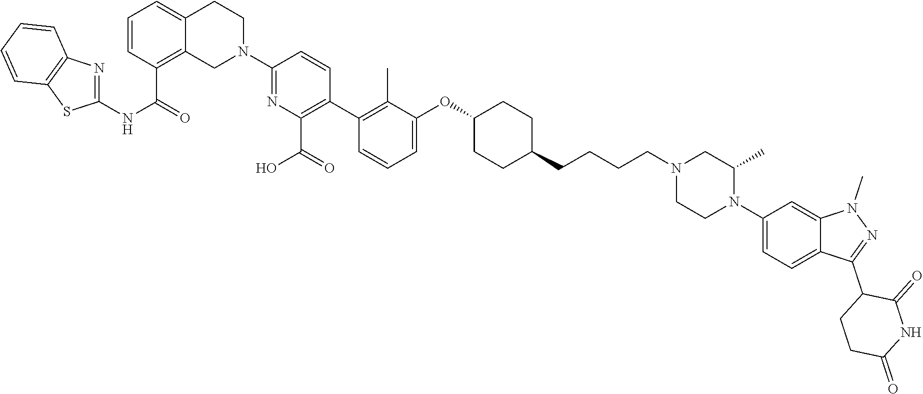 [SMILES: Cc1c(O[C@H]2CC[C@H](CCCCN3CCN(c4ccc5c(C6CCC(=O)NC6=O)nn(C)c5c4)[C@@H](C)C3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-(4-((3S)-4-(3-(2,6-dioxopiperidin-3-yl)-1-methyl-1H-indazol-6-yl)-3-methylpiperazin-1-yl)butyl)cyclohexyl)oxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(((1r,4r)-4-(4-((3S)-4-(3-(2,6-dioxopiperidin-3-yl)-1-methyl-1H-indazol-6-yl)-3-methylpiperazin-1-yl)butyl)cyclohexyl)oxy)-2-methylphenyl)picolinate

To a solution of tert-butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate (109.10 mg, 146.45 mol, 1 equiv.) and 3-[1-methyl-6-[(2S)-2-methylpiperazin-1-yl]indazol-3-yl]piperidine-2,6-dione (50 mg, 146.45 μmol, 1 equiv.) in DCM (3 mL) was added NaBH(OAc)₃ (186.24 mg, 878.72 mol, 6 equiv.). The mixture was stirred at 25° C. for 12 hours. The reaction mixture was filtered and concentrated under reduced pressure to give a residue. The residue was purified by prep-TLC (SiO₂, DCM:MeOH=10:1) to give tert-butyl 6-(8-(benzo[d]thiazol-2-ylcarbamoyl)-3,4-dihydroisoquinolin-2(1H)-yl)-3-(3-(((1r,4r)-4-(4-((3S)-4-(3-(2,6-dioxopiperidin-3-yl)-1-methyl-1H-indazol-6-yl)-3-methylpiperazin-1-yl)butyl)cyclohexyl)oxy)-2-methylphenyl)picolinate (60 mg, 53.34 μmol, 36.4% yield) as a brown solid.

### Step B. Procedure for Preparation of 6-(8-(benzo[d]thiazol-2-ylcarbamoyl)-3,4-dihydroisoquinolin-2(1H)-yl)-3-(3-(((1r,4r)-4-(4-((3S)-4-(3-(2,6-dioxopiperidin-3-yl)-1-methyl-1H-indazol-6-yl)-3-methylpiperazin-1-yl)butyl)cyclohexyl)oxy)-2-methylphenyl)picolinic acid

A solution of tert-butyl 6-(8-(benzo[d]thiazol-2-ylcarbamoyl)-3,4-dihydroisoquinolin-2(1H)-yl)-3-(3-(((1r,4r)-4-(4-((3S)-4-(3-(2,6-dioxopiperidin-3-yl)-1-methyl-1H-indazol-6-yl)-3-methylpiperazin-1-yl)butyl)cyclohexyl)oxy)-2-methylphenyl)picolinate (60 mg, 56.06 μmol, 1 equiv.) in DCM (2 mL) and TFA (2 mL) was stirred at 25° C. for 16 hour. The reaction mixture was concentrated under reduced pressure to give a residue. The residue was purified by reverse-phase HPLC (with 0.1% formic acid) to give 6-(8-(benzo[d]thiazol-2-ylcarbamoyl)-3,4-dihydroisoquinolin-2(1H)-yl)-3-(3-(((1r,4r)-4-(4-((3S)-4-(3-(2,6-dioxopiperidin-3-yl)-1-methyl-1H-indazol-6-yl)-3-methylpiperazin-1-yl)butyl)cyclohexyl)oxy)-2-methylphenyl)picolinic acid (37.55 mg, 36.92 μmol, 65.8% yield) as a white solid.

MS (ESI) m/z: 1014.6 [M+H]⁺.

¹H NMR (400 MHz, DMSO-d₆) δ=10.84 (s, 1H), 8.16 (s, 1H), 8.03 (d, J=7.6 Hz, 1H), 7.78 (d, J=8.0 Hz, 1H), 7.62 (d, J=7.6 Hz, 1H), 7.53-7.40 (m, 4H), 7.40-7.31 (m, 2H), 7.09-7.03 (m, 1H), 6.96-6.85 (m, 3H), 6.77 (s, 1H), 6.64-6.58 (m, 1H), 5.01-4.91 (m, 2H), 4.29-4.15 (m, 2H), 4.14-4.04 (m, 1H), 3.96-3.84 (m, 5H), 3.04-3.01 (m, 2H), 2.90-2.84 (m, 1H), 2.71 (d, J=11.2 Hz, 1H), 2.66-2.59 (m, 2H), 2.28 (d, J=7.6 Hz, 5H), 2.18-2.03 (m, 5H), 1.93-1.86 (m, 3H), 1.82-1.74 (m, 2H), 1.48-1.42 (m, 2H), 1.39-1.22 (m, 7H), 1.10-0.98 (m, 5H)

### Example 238. Preparation of Compound 297b

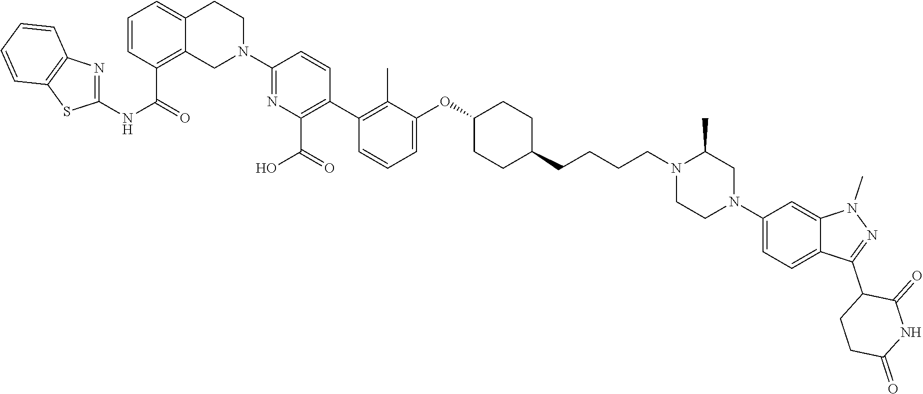 [SMILES: Cc1c(O[C@H]2CC[C@H](CCCCN3CCN(c4ccc5c(C6CCC(=O)NC6=O)nn(C)c5c4)C[C@@H]3C)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-(4-((2S)-4-(3-(2,6-dioxopiperidin-3-yl)-1-methyl-1H-indazol-6-yl)-2-methylpiperazin-1-yl)butyl)cyclohexyl)oxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(((1r,4r)-4-(4-((2S)-4-(3-(2,6-dioxopiperidin-3-yl)-1-methyl-1H-indazol-6-yl)-2-methylpiperazin-1-yl)butyl)cyclohexyl)oxy)-2-methylphenyl)picolinate

A mixture of tert-butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate (100 mg, 134.24 μmol, 1.0 equiv.), and 3-[1-methyl-6-[(3S)-3-methylpiperazin-1-yl]indazol-3-yl]piperidine-2,6-dione (60.87 mg, 161.09 μmol, 1.2 equiv., HCl) in DCM (2 mL) was stirred at 25° C. for 1 hour. To this reaction mixture was added NaBH(OAc)₃ (142.25 mg, 671.20 μmol, 5.0 equiv.), and the mixture was stirred at 25° C. for 3 hours. The reaction mixture was diluted with water (40 mL) and extracted with DCM (40 mL×3). The combined organic layers were washed with brine (40 mL×2), dried over Na₂SO₄, filtered, and concentrated under reduced pressure to give tert-butyl 6-(8-(benzo[d]thiazol-2-ylcarbamoyl)-3,4-dihydroisoquinolin-2(1H)-yl)-3-(3-(((1r,4r)-4-(4-((2S)-4-(3-(2,6-dioxopiperidin-3-yl)-1-methyl-1H-indazol-6-yl)-2-methylpiperazin-1-yl)butyl)cyclohexyl)oxy)-2-methylphenyl)picolinate (115 mg, 107.4 μmol, 80.0% yield) as a yellow solid.

MS (ESI) m/z: 536.1 [M/2+H]⁺.

### Step B. Procedure for Preparation of 6-(8-(benzo[d]thiazol-2-ylcarbamoyl)-3,4-dihydroisoquinolin-2(1H)-yl)-3-(3-(((1r,4r)-4-(4-((2S)-4-(3-(2,6-dioxopiperidin-3-yl)-1-methyl-1H-indazol-6-yl)-2-methylpiperazin-1-yl)butyl)cyclohexyl)oxy)-2-methylphenyl)picolinic acid

A solution of tert-butyl 6-(8-(benzo[d]thiazol-2-ylcarbamoyl)-3,4-dihydroisoquinolin-2(1H)-yl)-3-(3-(((1r,4r)-4-(4-((2S)-4-(3-(2,6-dioxopiperidin-3-yl)-1-methyl-1H-indazol-6-yl)-2-methylpiperazin-1-yl)butyl)cyclohexyl)oxy)-2-methylphenyl)picolinate (115.00 mg, 107.44 mol, 1.0 equiv.), DCM (1.5 mL), and TFA (0.5 mL) was stirred at 25° C. for 12 hours. The reaction mixture was concentrated under reduced pressure to remove TFA and DCM to give a residue. The residue was purified by prep-HPLC to give 6-(8-(benzo[d]thiazol-2-ylcarbamoyl)-3,4-dihydroisoquinolin-2(1H)-yl)-3-(3-(((1r,4r)-4-(4-((2S)-4-(3-(2,6-dioxopiperidin-3-yl)-1-methyl-1H-indazol-6-yl)-2-methylpiperazin-1-yl)butyl)cyclohexyl)oxy)-2-methylphenyl)picolinic acid (18.53 mg, 17.26 μmol, 16.0% yield) was obtained as an off-white solid.

¹H NMR (400 MHz, DMSO-d₆) δ=10.85 (s, 1H), 8.18 (s, 1H), 8.03 (d, J=8.0 Hz, 1H), 7.78 (d, J=8.0 Hz, 1H), 7.66-7.59 (m, 1H), 7.50-7.41 (m, 4H), 7.39-7.32 (m, 2H), 7.10-7.01 (m, 1H), 6.95-6.87 (m, 3H), 6.85-6.79 (m, 1H), 6.62 (m, 1H), 4.97 (s, 2H), 4.25 (m, 1H), 4.21-4.13 (m, 1H), 3.93-3.86 (m, 5H), 3.56-3.48 (m, 3H), 3.08-2.96 (m, 3H), 2.93-2.84 (m, 2H), 2.71 (m, 1H), 2.62-2.59 (m, 2H), 2.25-2.13 (m, 3H), 2.10-2.04 (m, 2H), 1.93 (s, 1H), 1.87 (s, 3H), 1.81-1.73 (m, 2H), 1.46-1.18 (m, 11H), 1.08 (m, 3H).

### Example 239. Preparation of Compound 302

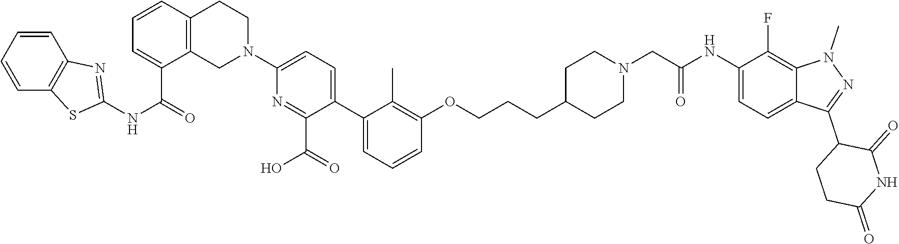 [SMILES: Cc1c(OCCCC2CCN(CC(=O)Nc3ccc4c(C5CCC(=O)NC5=O)nn(C)c4c3F)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-7-fluoro-1-methyl-indazol-6-yl]amino]-2-oxo-ethyl]-4-piperidyl]propoxy]-2-methyl-phenyl]pyridine-2-carboxylic acid

### Step A. Procedure for Preparation of (E)-4-bromo-2,3-difluorobenzaldehyde O-methyl oxime

To a solution of O-methylhydroxylamine (6.24 g, 74.66 mmol, 1.1 equiv., HCl) in DME (150 mL) was added K₂CO₃ (28.14 g, 203.62 mmol, 3.0 equiv.) and 4-bromo-2,3-difluoro-benzaldehyde (15.0 g, 67.87 mmol, 1.0 equiv.). The mixture was stirred at 40° C. for 3 hours. The reaction mixture was filtered and concentrated under reduced pressure to give (E)-4-bromo-2,3-difluorobenzaldehyde O-methyl oxime (52 g, crude) as a white solid.

### Step B. Procedure for Preparation of 6-bromo-7-fluoro-1H-indazole

To a solution of (E)-4-bromo-2,3-difluorobenzaldehyde O-methyl oxime (26 g, 103.98 mmol, 1.0 equiv.) in DME (260 mL) was added N₂H₄·H₂O (19.42 g, 380.17 mmol, 18.82 mL, 98% purity, 3.66 equiv.). The mixture was stirred at 90° C. for 16 hours. The reaction mixture was cooled to 20° C., concentrated under reduced pressure, added into H₂O, filtered, and concentrated under reduced pressure to give a residue. The residue was triturated with petroleum ether at 20° C. for 10 min to give 6-bromo-7-fluoro-1H-indazole (44.0 g, 193.70 mmol, 93.1% yield) as a white solid.

MS (ESI) m/z: 215.2 [M+H]⁺

¹H NMR (400 MHz, DMSO-d₆) δ=8.22 (d, J=3.4 Hz, 1H), 7.56 (d, J=8.5 Hz, 1H), 7.29 (dd, J=5.8, 8.5 Hz, 1H)

¹⁹F NMR (377 MHz, DMSO-d₆) δ=−125.15 (s, 1F)

### Step C. Procedure for Preparation of 6-bromo-7-fluoro-3-iodo-1H-indazole

To a solution of 6-bromo-7-fluoro-1H-indazole (44 g, 204.63 mmol, 1.0 equiv.) in DMF (400 mL) was added KOH (22.96 g, 409.26 mmol, 2.0 equiv.) and 12 (83.10 g, 327.41 mmol, 65.95 mL, 1.6 equiv.). The mixture was stirred at 25° C. for 16 hours. The reaction mixture was quenched by addition of saturated Na₂SO₃ solution (20 mL) at 20° C., diluted with H₂O, filtered, and concentrated under reduced pressure to give a residue. The residue was triturated with H₂O at 20° C. for 10 min to give 6-bromo-7-fluoro-3-iodo-1H-indazole (49.0 g, 134.96 mmol, 65.9% yield) as a light brown solid.

MS (ESI) m/z: 341.0 [M+H]⁺

¹H NMR (400 MHz, DMSO-d₆) δ=7.40-7.34 (m, 1H), 7.25-7.20 (m, 1H)

¹⁹F NMR (377 MHz, DMSO-d₆) δ=−124.27 (s, 1F)

### Step D. Procedure for Preparation of 6-bromo-7-fluoro-3-iodo-1-methyl-indazole

To a solution of 6-bromo-7-fluoro-3-iodo-1H-indazole (49.0 g, 143.73 mmol, 1.0 equiv.) in DMF (500 mL) was added K₂CO₃ (59.59 g, 431.19 mmol, 3.0 equiv.) and Mel (40.80 g, 287.46 mmol, 17.90 mL, 2.0 equiv.). The mixture was stirred at 40° C. for 16 hours. The reaction mixture was quenched by addition of saturated NH₄Cl solution (100 mL) at 0° C., diluted with H₂O, and extracted with EtOAc (1000 mL×3). The combined organic layers were washed with H₂O (3000 mL×3), dried over Na₂SO₄, filtered, and concentrated under reduced pressure to give a residue. The residue was purified by column chromatography (SiO₂, petroleum ether/ethyl acetate=200/1 to 100/1) to give 6-bromo-7-fluoro-3-iodo-1-methyl-indazole (29.3 g, 81.75 mmol, 56.8% yield) as a white solid.

MS (ESI) m/z: 357.0 [M+H]⁺

¹H NMR (400 MHz, DMSO-d₆) δ=7.42-7.34 (m, 1H), 7.21 (d, J=8.8 Hz, 1H), 4.21-4.13 (m, 3H)

¹⁹F NMR (377 MHz, DMSO-d₆) δ=−126.90 (s, 1F)

### Step E. Procedure for Preparation of 3-(2,6-bis(benzyloxy)pyridin-3-yl)-6-bromo-7-fluoro-1-methyl-1H-indazole

A mixture of 6-bromo-7-fluoro-3-iodo-1-methyl-indazole (29.3 g, 82.55 mmol, 1.0 equiv.), 2,6-dibenzyloxy-3-(4,4,5,5-tetramethyl-1,3,2-dioxaborolan-2-yl)pyridine (37.89 g, 90.80 mmol, 1.1 equiv.), Cs₂CO₃ (80.69 g, 247.64 mmol, 3.0 equiv.), and Pd(dppf)Cl₂·CH₂Cl₂ (3.37 g, 4.13 mmol, 0.05 equiv.) in THE (300 mL) and H₂O (30 mL) was degassed and purged with N₂ three times, and then the mixture was stirred at 60° C. for 16 hours under N₂ atmosphere. The mixture was filtered and concentrated under reduced pressure to give a residue. The residue was purified by column chromatography (SiO₂, petroleum ether/ethyl acetate=200/1 to 50/1) to give 3-(2,6-bis(benzyloxy)pyridin-3-yl)-6-bromo-7-fluoro-1-methyl-1H-indazole (30.35 g, 53.95 mmol, 65.3% yield) as a light yellow solid.

MS (ESI) m/z: 520.2 [M+H]⁺

¹H NMR (400 MHz, CDCl₃) δ=7.82 (d, J=8.0 Hz, 1H), 7.42-7.38 (m, 2H), 7.36-7.29 (m, 3H), 7.27-7.19 (m, 6H), 6.99 (dd, J=5.6, 8.4 Hz, 1H), 6.48 (d, J=8.0 Hz, 1H), 5.40 (s, 2H), 5.37 (s, 2H), 4.23-4.18 (m, 3H)

¹⁹F NMR (377 MHz, CDCl₃) δ=−128.97 (s, 1F)

### Step F. Procedure for Preparation of N-benzyl-3-(2,6-dibenzyloxy-3-pyridyl)-7-fluoro-1-methyl-indazol-6-amine

A mixture of 3-(2,6-bis(benzyloxy)pyridin-3-yl)-6-bromo-7-fluoro-1-methyl-1H-indazole (5 g, 9.65 mmol, 1.0 equiv.), phenylmethanamine (1.55 g, 14.47 mmol, 1.58 mL, 1.5 equiv.), Cs₂CO₃ (9.43 g, 28.94 mmol, 3.0 equiv.), and tBuBrettPhos Pd G₃ (412.07 mg, 482.28 μmol, 0.05 equiv.) in 2-methylbutan-2-ol (30 mL) was degassed and purged with N₂ three times. The mixture was stirred at 80° C. for 16 hours under N₂ atmosphere. The mixture was filtered and concentrated under reduced pressure to give a residue. The residue was purified by flash silica gel chromatography (0˜10% ethyl acetate/petroleum ether) to give N-benzyl-3-(2,6-dibenzyloxy-3-pyridyl)-7-fluoro-1-methyl-indazol-6-amine (8.5 g, 15.46 mmol, 80.1% yield) as a yellow oil.

MS (ESI) m/z: 545.5 [M+H]⁺

¹H NMR (400 MHz, DMSO-d₆) δ=7.83 (d, J=8.0 Hz, 1H), 7.48-7.43 (m, 2H), 7.41-7.27 (m, 9H), 7.26-7.16 (m, 4H), 7.14 (d, J=8.8 Hz, 1H), 6.55 (d, J=8.0 Hz, 1H), 6.49 (dd, J=7.2, 8.8 Hz, 1H), 6.37-6.31 (m, 1H), 5.39 (d, J=5.6 Hz, 4H), 4.43 (d, J=6.4 Hz, 2H), 4.14-4.05 (m, 3H)

¹⁹F NMR (377 MHz, DMSO-d₆) δ=−162.61 (s, 1F)

### Step G. Procedure for Preparation of 3-(6-amino-7-fluoro-1-methyl-indazol-3-yl)piperidine-2,6-dione

To a solution of N-benzyl-3-(2,6-dibenzyloxy-3-pyridyl)-7-fluoro-1-methyl-indazol-6-amine (2.0 g, 3.67 mmol, 1.0 equiv.) in THE (40 mL) was added Pd/C (1.00 g, 939.67 μmol, 10% purity, 0.256 equiv.) and Pd(OH)₂ (1.00 g, 1.42 mmol, 20% purity, 0.388 equiv.) under N₂. The suspension was degassed under vacuum and purged with H₂ several times. The mixture was stirred under H₂ (15 psi) at 20° C. for 3 hours. The mixture was filtered and concentrated under reduced pressure to give a residue. The residue was purified by reverse-phase HPLC (with 0.05% formic acid) to give 3-(6-amino-7-fluoro-1-methyl-indazol-3-yl)piperidine-2,6-dione (1336.60 mg, 4.39 mmol, 87.8% yield) as a gray solid.

MS (ESI) m/z: 277.2 [M+H]⁺

¹H NMR (400 MHz, DMSO-d₆) δ=10.84 (s, 1H), 7.17 (d, J=8.8 Hz, 1H), 6.63 (t, J=8.0 Hz, 1H), 5.34-5.26 (m, 2H), 4.25-4.18 (m, 1H), 3.99 (s, 3H), 2.64-2.58 (m, 2H), 2.31-2.22 (m, 1H), 2.19-2.10 (m, 1H)

¹⁹F NMR (377 MHz, DMSO-d₆) δ=−163.31 (s, 1F)

### Step H. Procedure for Preparation of tert-butyl 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-7-fluoro-1-methyl-indazol-6-yl]amino]-2-oxo-ethyl]-4-piperidyl]propoxy]-2-methyl-phenyl]pyridine-2-carboxylate

To a solution of 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-1-piperidyl]acetic acid (118 mg, 152.07 μmol, 1 equiv.) and 3-(6-amino-7-fluoro-1-methyl-indazol-3-yl)piperidine-2,6-dione (50.41 mg, 182.49 μmol, 1.2 equiv.) in pyridine (1 mL) was added EDCI (43.73 mg, 228.11 μmol, 1.5 equiv.). The mixture was stirred at 25° C. for 2 hours. The reaction mixture was then added dropwise into water, and then filtered and concentrated to give tert-butyl 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-7-fluoro-1-methyl-indazol-6-yl]amino]-2-oxo-ethyl]-4-piperidyl]propoxy]-2-methyl-phenyl]pyridine-2-carboxylate (137 mg, 132.4 μmol, 87.1% yield) as a brown solid.

MS (ESI) m/z: 518.2 [M/2+H]⁺.

### Step I. Procedure for preparation of 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-7-fluoro-1-methyl-indazol-6-yl]amino]-2-oxo-ethyl]-4-piperidyl]propoxy]-2-methyl-phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-7-fluoro-1-methyl-indazol-6-yl]amino]-2-oxo-ethyl]-4-piperidyl]propoxy]-2-methyl-phenyl]pyridine-2-carboxylate (137 mg, 132.47 μmol, 1 equiv.) in TFA (1 mL) and DCM (3 mL) was stirred at 25° C. for 2 hours. The residue was concentrated under reduced pressure, and the resulting residue was purified by prep-HPLC to give 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-7-fluoro-1-methyl-indazol-6-yl]amino]-2-oxo-ethyl]-4-piperidyl]propoxy]-2-methyl-phenyl]pyridine-2-carboxylic acid (38.1 mg, 38.95 μmol, 29.4% yield) as an off-white solid.

MS (ESI) m/z: 978.7 [M+H]⁺.

1H NMR (400 MHz, DMSO-d₆) δ=12.88 (dd, J=3.2, 6.0 Hz, 1H), 10.92 (s, 1H), 9.79-9.62 (m, 1H), 8.03 (d, J=8.4 Hz, 1H), 7.79 (d, J=8.0 Hz, 1H), 7.65-7.53 (m, 2H), 7.51-7.31 (m, 7H), 7.09 (t, J=8.0 Hz, 1H), 6.97 (d, J=8.8 Hz, 1H), 6.88 (d, J=8.4 Hz, 1H), 6.63 (d, J=7.6 Hz, 1H), 4.98 (s, 2H), 4.37 (dd, J=5.2, 10.4 Hz, 1H), 4.10 (s, 3H), 4.00-3.94 (m, 2H), 3.92 (t, J=5.6 Hz, 2H), 3.15 (d, J=2.4 Hz, 2H), 3.04-3.01 (m, 2H), 2.95-2.88 (m, 2H), 2.20-2.14 (m, 4H), 1.90 (s, 3H), 1.83-1.70 (m, 5H), 1.45-1.38 (m, 2H), 1.28 (dd, J=4.8, 13.2 Hz, 3H).

### Example 240. Preparation of Compound 303

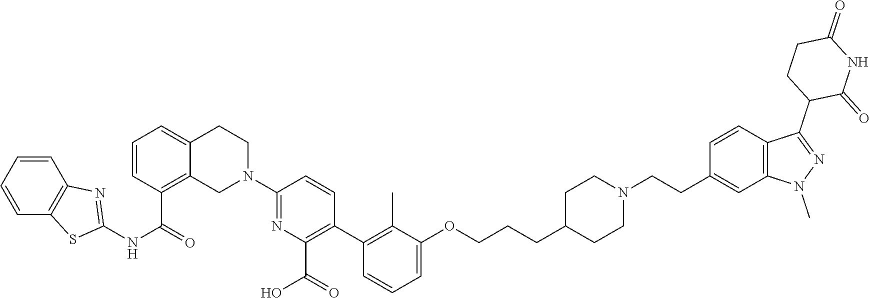 [SMILES: Cc1c(OCCCC2CCN(CCc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-(1-(2-(3-(2,6-dioxopiperidin-3-yl)-1-methyl-1H-indazol-6-yl)ethyl)piperidin-4-yl)propoxy)-2-methylphenyl)picolinic acid

### Step A. Procedure for Preparation of (E)-3-(2, 6-bis(benzyloxy)pyridin-3-yl)-6-(2-ethoxyvinyl)-1-methyl-1H-indazole

A mixture of 3-(2, 6-bis(benzyloxy)pyridin-3-yl)-6-bromo-1-methyl-1H-indazole (3 g, 6.00 mmol, 1 equiv.), (E)-2-(2-ethoxyvinyl)-4, 4, 5, 5-tetramethyl-1, 3, 2-dioxaborolane (1.42 g, 7.19 mmol, 1.2 equiv.), K₃PO₄ (3.18 g, 14.99 mmol, 2.5 equiv.), Pd(OAc)₂ (67.30 mg, 299.77 μmol, 0.05 equiv.), and dicyclohexyl-[2-(2, 6-dimethoxyphenyl)phenyl]phosphane (2.46 g, 6.00 mmol, 1 equiv.) in MeCN (30 ml) and H₂O (10 ml) was degassed and purged with N₂ three times. The mixture was stirred at 60° C. for 2 hours under N₂ atmosphere. The reaction was concentrated under reduced pressure to give a residue. The residue was purified by flash silica gel chromatography (0˜ 20% ethyl acetate/petroleum ether) to give (E)-3-(2, 6-bis(benzyloxy)pyridin-3-yl)-6-(2-ethoxyvinyl)-1-methyl-1H-indazole (2.8 g, 5.70 mmol, 95.0% yield) as a yellow oil.

MS (ESI) m/z: 492.3 [M+H]⁺.

### Step B. Procedure for Preparation of 2-(3-(2, 6-bis(benzyloxy)pyridin-3-yl)-1-methyl-1H-indazol-6-yl)acetaldehyde

A mixture of (E)-3-(2, 6-bis(benzyloxy)pyridin-3-yl)-6-(2-ethoxyvinyl)-1-methyl-1H-indazole (2.8 g, 5.70 mmol, 1 equiv.) in HCOOH (30 mL) was stirred at 40° C. for 2 hours. The reaction mixture was filtered and concentrated under reduced pressure to give 2-(3-(2, 6-bis(benzyloxy)pyridin-3-yl)-1-methyl-1H-indazol-6-yl)acetaldehyde (2.5 g, crude) as a yellow solid.

MS (ESI) m/z: 464.2 [M+H]⁺.

### Step C. Procedure for Preparation of 3-(2, 6-bis(benzyloxy)pyridin-3-yl)-6-(2, 2-dimethoxyethyl)-1-methyl-1H-indazole

A mixture of 2-(3-(2, 6-bis(benzyloxy)pyridin-3-yl)-1-methyl-1H-indazol-6-yl)acetaldehyde (2.5 g, 5.39 mmol, 1 equiv.) and TsOH (92.88 mg, 539.34 μmol, 0.1 equiv.) in MeOH (10 ml) and toluene (20 ml) was degassed and purged with N₂ three times. The mixture was stirred at 130° C. for 12 hours under N₂ atmosphere. The reaction mixture was filtered and concentrated under reduced pressure to give a residue. The residue was purified by prep-HPLC to give 3-(2, 6-bis(benzyloxy)pyridin-3-yl)-6-(2, 2-dimethoxyethyl)-1-methyl-1H-indazole (460 mg, 902.6 μmol, 16.7% yield) as a yellow oil.

MS (ESI) m/z: 510.4 [M+H]⁺.

### Step D. Procedure for Preparation of 3-(6-(2, 2-dimethoxyethyl)-1-methyl-1H-indazol-3-yl)piperidine-2, 6-dione

A mixture of 3-(2, 6-bis(benzyloxy)pyridin-3-yl)-6-(2, 2-dimethoxyethyl)-1-methyl-1H-indazole (460.00 mg, 902.68 μmol, 1 equiv.), Pd/C (960.63 mg, 902.68 μmol, 10% purity, 1 equiv.), and Pd(OH)₂ (1.27 g, 902.68 μmol, 10% purity, 1 equiv.) in THE (8 ml) was stirred at 50° C. for 12 hours under H₂ atmosphere. The reaction mixture was filtered, and the filtrate was concentrated under reduced pressure to give 3-(6-(2, 2-dimethoxyethyl)-1-methyl-1H-indazol-3-yl)piperidine-2, 6-dione (330 mg, crude) as a yellow solid.

MS (ESI) m/z: 332.2 [M+H]⁺.

¹H NMR (400 MHz, DMSO-d₆) δ=10.88 (s, 1H), 7.60 (d, J=8.4 Hz, 1H), 7.44 (s, 1H), 7.02 (dd, J=1.2, 8.4 Hz, 1H), 4.64 (t, J=5.6 Hz, 1H), 4.34 (dd, J=4.8, 10.0 Hz, 1H), 3.96 (s, 3H), 3.25 (s, 6H), 2.98 (d, J=5.6 Hz, 2H), 2.71-2.60 (m, 2H), 2.37-2.16 (m, 2H)

### Step E. Procedure for Preparation of 2-(3-(2, 6-dioxopiperidin-3-yl)-1-methyl-1H-indazol-6-yl)acetaldehyde

A mixture of 3-(6-(2, 2-dimethoxyethyl)-1-methyl-1H-indazol-3-yl)piperidine-2, 6-dione (100 mg, 301.78 μmol, 1 equiv.) in HCOOH (2 ml) was stirred at 100° C. for 1 hour. The reaction mixture was filtered and concentrated under reduced pressure to give 2-(3-(2, 6-dioxopiperidin-3-yl)-1-methyl-1H-indazol-6-yl)acetaldehyde (70 mg, crude) as a green solid.

MS (ESI) m/z: 286.2 [M+H]⁺.

### Step F. Procedure for Preparation of 6-(8-(benzo[d]thiazol-2-ylcarbamoyl)-3,4-dihydroisoquinolin-2(1H)-yl)-3-(3-(3-(1-(2-(3-(2,6-dioxopiperidin-3-yl)-1-methyl-1H-indazol-6-yl)ethyl)piperidin-4-yl)propoxy)-2-methylphenyl)picolinic acid

A mixture of 6-(8-(benzo[d]thiazol-2-ylcarbamoyl)-3,4-dihydroisoquinolin-2(1H)-yl)-3-(2-methyl-3-(3-(piperidin-4-yl)propoxy)phenyl)picolinic acid (150 mg, 226.65 μmol, 1 equiv.), 2-(3-(2, 6-dioxopiperidin-3-yl)-1-methyl-1H-indazol-6-yl)acetaldehyde (71.13 mg, 249.32 μmol, 1.1 equiv.), and 4A MS (30 mg) in DCM (4 ml) was stirred at 25° C. for 12 hours. Afterwards, NaBH(OAc)₃ (96.07 mg, 453.30 μmol, 2 equiv.) was added into the mixture, and then the mixture was stirred at 25° C. for 0.5 hour. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(3-(1-(2-(3-(2,6-dioxopiperidin-3-yl)-1-methyl-1H-indazol-6-yl)ethyl)piperidin-4-yl)propoxy)-2-methylphenyl)picolinic acid (29.55 mg, 28.42 μmol, 12.54% yield) as a yellow solid.

MS (ESI) m/z: 931.6 [M+H]⁺.

¹H NMR (400 MHz, DMSO-d₆) δ=10.88 (s, 1H), 8.16 (s, 1H), 8.01 (d, J=7.6 Hz, 1H), 7.78 (d, J=8.0 Hz, 1H), 7.64-7.59 (m, 3H), 7.47-7.42 (m, 3H), 7.40-7.34 (m, 2H), 7.09 (t, J=8.0 Hz, 1H), 7.00 (d, J=8.0 Hz, 1H), 6.93 (d, J=8.8 Hz, 1H), 6.87 (d, J=8.0 Hz, 1H), 6.64 (d, J=7.6 Hz, 1H), 4.98 (s, 2H), 4.33 (dd, J=5.2, 9.6 Hz, 1H), 3.98-3.90 (m, 7H), 3.02 (d, J=4.8 Hz, 2H), 2.94-2.88 (m, 2H), 2.76-2.68 (m, 2H), 2.63 (dd, J=6.0, 11.6 Hz, 2H), 2.22-2.09 (m, 4H), 1.90 (s, 3H), 1.79-1.66 (m, 4H), 1.46-1.25 (m, 4H), 1.24-1.03 (m, 3H)

### Example 241. Preparation of Compound 304

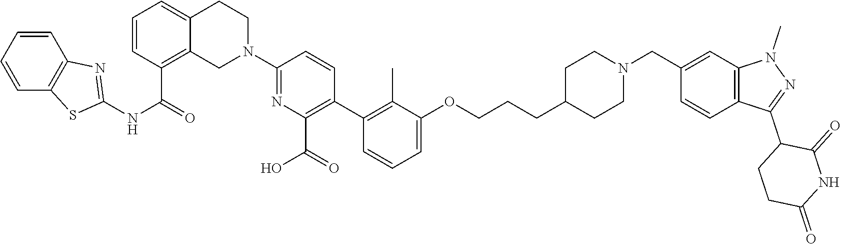 [SMILES: Cc1c(OCCCC2CCN(Cc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-(1-((3-(2,6-dioxopiperidin-3-yl)-1-methyl-1H-indazol-6-yl)methyl)piperidin-4-yl)propoxy)-2-methylphenyl)picolinic acid

### Step A. Procedure for Preparation of 3-(2,6-bis(benzyloxy)pyridin-3-yl)-1-methyl-6-vinyl-1H-indazole

A mixture of 3-(2,6-bis(benzyloxy)pyridin-3-yl)-6-bromo-1-methyl-1H-indazole (5 g, 9.99 mmol, 1 equiv.), trifluoro(vinyl)-14-borane potassium salt (1.34 g, 9.99 mmol, 1 equiv.), Pd(dppf)Cl₂ (219.34 mg, 299.77 μmol, 0.03 equiv.), and K₃PO₄ (3.18 g, 14.99 mmol, 1.5 equiv.) in dioxane (27.3 mL) and H₂O (2.7 mL) was degassed and purged with N₂ three times. The mixture was stirred at 80° C. for 2 hours under N₂ atmosphere. The reaction was filtered and concentrated under reduced pressure to give a residue. The residue was purified by flash silica gel chromatography (0˜15% ethyl acetate/petroleum ether) to give 3-(2,6-bis(benzyloxy)pyridin-3-yl)-1-methyl-6-vinyl-1H-indazole (4.4 g, 9.83 mmol, 98.39% yield) as a yellow oil.

MS (ESI) m/z: 448.2 [M+H]⁺

### Step B. Procedure for Preparation of 1-(3-(2,6-bis(benzyloxy)pyridin-3-yl)-1-methyl-1H-indazol-6-yl)ethane-1,2-diol

A mixture of 3-(2,6-bis(benzyloxy)pyridin-3-yl)-1-methyl-6-vinyl-1H-indazole (1.90 g, 4.25 mmol, 1 equiv.), K₂OsO₄.2H₂O (156.43 mg, 424.56 μmol, 0.1 equiv.), and NMO (1.49 g, 12.74 mmol, 1.34 mL, 3 equiv.) in acetone (35 mL) and H₂O (3.5 mL) was degassed and purged with N₂ three times. The mixture was stirred at 65° C. for 2 hours under N₂ atmosphere. The reaction was concentrated under reduced pressure to give a residue. The residue was purified by flash silica gel chromatography (0˜66% ethyl acetate/petroleum ether) to give 1-(3-(2,6-bis(benzyloxy)pyridin-3-yl)-1-methyl-1H-indazol-6-yl)ethane-1,2-diol (2.2 g, 4.57 mmol, 53.81% yield) as a yellow oil.

MS (ESI) m/z: 482.3 [M+H]⁺

### Step C. Procedure for Preparation of 3-(2,6-bis(benzyloxy)pyridin-3-yl)-1-methyl-1H-indazole-6-carbaldehyde

A mixture of 1-(3-(2,6-bis(benzyloxy)pyridin-3-yl)-1-methyl-1H-indazol-6-yl)ethane-1,2-diol (2.2 g, 4.57 mmol, 1 equiv.) and NaIO₄ (1.95 g, 9.14 mmol, 506.32 μL, 2 equiv.) in acetone (20 mL) and H₂O (2 mL) was stirred at 25° C. for 2 hours. The mixture was quenched by sat. Na₂SO₃ (20 mL) and exacted with DCM (30 mL, 10 mL×3). The combined organic phase was concentrated to a residue. The residue was purified by flash silica gel chromatography (0˜30% ethyl acetate/petroleum ether) to give 3-(2,6-bis(benzyloxy)pyridin-3-yl)-1-methyl-1H-indazole-6-carbaldehyde (2 g, 4.45 mmol, 97.39% yield) as a yellow oil.

MS (ESI) m/z: 450.2 [M+H]⁺

### Step D. Procedure for Preparation of 3-(2,6-bis(benzyloxy)pyridin-3-yl)-6-(1,3-dioxolan-2-yl)-1-methyl-1H-indazole

A mixture of 3-(2,6-bis(benzyloxy)pyridin-3-yl)-1-methyl-1H-indazole-6-carbaldehyde (700 mg, 1.56 mmol, 1 equiv.), ethylene glycol (483.30 mg, 7.79 mmol, 434.24 μL, 5 equiv.), and TsOH (26.82 mg, 155.73 μmol, 0.1 equiv.) in toluene (15 mL) was degassed and purged with N₂ three times. The mixture was stirred at 120° C. for 12 hours under N₂ atmosphere. The reaction was filtered and concentrated under reduced pressure to give 3-(2,6-bis(benzyloxy)pyridin-3-yl)-6-(1,3-dioxolan-2-yl)-1-methyl-1H-indazole (500 mg, 1.01 mmol, 65.05% yield) as a yellow solid.

MS (ESI) m/z: 494.2 [M+H]⁺.

### Step E. Procedure for Preparation of 3-(6-(1,3-dioxolan-2-yl)-1-methyl-1H-indazol-3-yl)piperidine-2,6-dione

A mixture of 3-(2,6-bis(benzyloxy)pyridin-3-yl)-6-(1,3-dioxolan-2-yl)-1-methyl-1H-indazole (500 mg, 1.01 mmol, 1 equiv.), Pd/C (600 mg, 563.80 μmol, 10% purity, 0.557 equiv.), and Pd(OH)₂ (600 mg, 427.23 μmol, 10% purity, 0.422 equiv.) in THF (15 mL) was degassed and purged with N₂ three times. The mixture was stirred at 25° C. for 12 hours under N₂ atmosphere. The reaction mixture was filtered and concentrated under reduced pressure to give a residue. The residue was purified by flash silica gel chromatography (0˜66% ethyl acetate/petroleum ether) to give 3-(6-(1,3-dioxolan-2-yl)-1-methyl-1H-indazol-3-yl)piperidine-2,6-dione (260 mg, 816.31 mol, 80.58% yield) as a yellow oil.

MS (ESI) m/z: 316.1 [M+H]⁺.

### Step F. Procedure for Preparation of 3-(2,6-dioxopiperidin-3-yl)-1-methyl-1H-indazole-6-carbaldehyde

A mixture of 3-(6-(1,3-dioxolan-2-yl)-1-methyl-1H-indazol-3-yl)piperidine-2,6-dione (100 mg, 317.13 μmol, 1 equiv.) in HCOOH (3 mL) was degassed and purged with N₂ three times, and then the mixture was stirred at 25° C. for 2 hours under N₂ atmosphere. The reaction mixture was filtered and concentrated under reduced pressure to give 3-(2,6-dioxopiperidin-3-yl)-1-methyl-1H-indazole-6-carbaldehyde (100 mg, crude) as a yellow oil.

MS (ESI) m/z: 272.0 [M+H]⁺.

### Step G. Procedure for Preparation of 6-(8-(benzo[d]thiazol-2-ylcarbamoyl)-3,4-dihydroisoquinolin-2(1H)-yl)-3-(3-(3-(1-((3-(2,6-dioxopiperidin-3-yl)-1-methyl-1H-indazol-6-yl)methyl)piperidin-4-yl)propoxy)-2-methylphenyl)picolinic acid

The pH of a mixture containing 3-(2,6-dioxopiperidin-3-yl)-1-methyl-1H-indazole-6-carbaldehyde (100 mg, 368.64 μmol, 1 equiv.), 6-(8-(benzo[d]thiazol-2-ylcarbamoyl)-3,4-dihydroisoquinolin-2(1H)-yl)-3-(2-methyl-3-(3-(piperidin-4-yl)propoxy)phenyl)picolinic acid (243.97 mg, 368.64 μmol, 1 equiv.), and DCM (3 mL) was adjusted to ˜6 with DIEA (0.5 mL). The mixture was stirred at 40° C. for 2 hours, and then NaBH(OAc)₃ (234.39 mg, 1.11 mmol, 3 equiv.) was added. The reaction mixture was then stirred at 40° C. for 0.5 hour. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(3-(1-((3-(2,6-dioxopiperidin-3-yl)-1-methyl-1H-indazol-6-yl)methyl)piperidin-4-yl)propoxy)-2-methylphenyl)picolinic acid (25.22 mg, 26.94 μmol, 7.31% yield) as a yellow solid.

MS (ESI) m/z: 917.8 [M+H]⁺.

¹H NMR (400 MHz, DMSO-d₆) δ=12.99-12.77 (m, 1H), 10.94-10.85 (m, 1H), 8.02 (d, J=8.0 Hz, 1H), 7.81-7.73 (m, 2H), 7.63 (d, J=7.6 Hz, 2H), 7.48-7.41 (m, 3H), 7.36 (td, J=7.6, 11.2 Hz, 2H), 7.18 (d, J=8.4 Hz, 1H), 7.09 (t, J=8.0 Hz, 1H), 6.96 (d, J=8.8 Hz, 1H), 6.87 (d, J=8.4 Hz, 1H), 6.63 (d, J=7.6 Hz, 1H), 4.98 (s, 2H), 4.38 (dd, J=5.2, 9.6 Hz, 1H), 4.00 (s, 3H), 3.98-3.88 (m, 4H), 3.03 (t, J=5.6 Hz, 2H), 2.75-2.59 (m, 4H), 2.40-2.31 (m, 4H), 2.21-2.14 (m, 1H), 1.89 (s, 3H), 1.85-1.69 (m, 4H), 1.52-1.20 (m, 6H)

### Example 242. Preparation of Compound 317

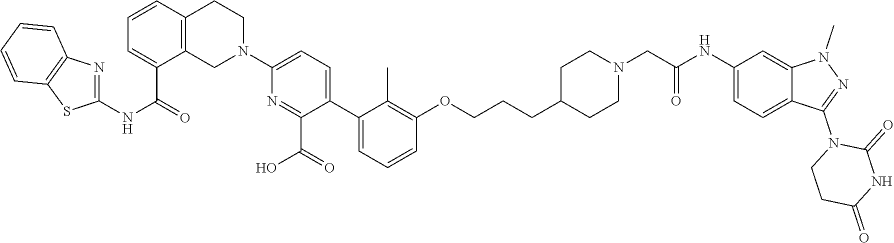 [SMILES: Cc1c(OCCCC2CCN(CC(=O)Nc3ccc4c(N5CCC(=O)NC5=O)nn(C)c4c3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-(1-(2-((3-(2,4-dioxotetrahydropyrimidin-1(2H)-yl)-1-methyl-1H-indazol-6-yl)amino)-2-oxoethyl)piperidin-4-yl)propoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(3-(1-(2-((3-(2,4-dioxotetrahydropyrimidin-1(2H)-yl)-1-methyl-1H-indazol-6-yl)amino)-2-oxoethyl)piperidin-4-yl)propoxy)-2-methylphenyl)picolinate

To a solution of 2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)piperidin-1-yl)acetic acid (100 mg, 128.87 μmol, 1 equiv.) in pyridine (3 mL) was added EDCI (32.12 mg, 167.54 μmol, 1.3 equiv.) and 1-(6-amino-1-methyl-1H-indazol-3-yl)dihydropyrimidine-2,4(1H,3H)-dione (43.4 mg, 167.54 μmol, 1.3 equiv.). The mixture was stirred at 40° C. for 1 hour. The reaction mixture was concentrated under reduced pressure followed by the addition of DCM (50 mL) and water (50 mL). The aqueous layer was washed with DCM, (15 mL×2), dried over anhydrous sodium sulfate, filtered, and concentrated under reduced pressure to give tert-butyl 6-(8-(benzo[d]thiazol-2-ylcarbamoyl)-3,4-dihydroisoquinolin-2(1H)-yl)-3-(3-(3-(1-(2-((3-(2,4-dioxotetrahydropyrimidin-1(2H)-yl)-1-methyl-1H-indazol-6-yl)amino)-2-oxoethyl)piperidin-4-yl)propoxy)-2-methylphenyl)picolinate (150 mg, 125.34 μmol, 97.26% yield) as a yellow oil.

MS (ESI) m/z: 1017.7 [M+H]⁺

### Step B. Procedure for Preparation of 6-(8-(benzo[d]thiazol-2-ylcarbamoyl)-3,4-dihydroisoquinolin-2(1H)-yl)-3-(3-(3-(1-(2-((3-(2,4-dioxotetrahydropyrimidin-1(2H)-yl)-1-methyl-1H-indazol-6-yl)amino)-2-oxoethyl)piperidin-4-yl)propoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(3-(1-(2-((3-(2,4-dioxotetrahydropyrimidin-1(2H)-yl)-1-methyl-1H-indazol-6-yl)amino)-2-oxoethyl)piperidin-4-yl)propoxy)-2-methylphenyl)picolinate (150 mg, 147.46 μmol, 1 equiv.) in DCM (1 mL) was added TFA (3.07 g, 26.93 mmol, 2 mL, 182.59 equiv.). The mixture was stirred at 40° C. for 1 hour. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(3-(1-(2-((3-(2,4-dioxotetrahydropyrimidin-1(2H)-yl)-1-methyl-1H-indazol-6-yl)amino)-2-oxoethyl)piperidin-4-yl)propoxy)-2-methylphenyl)picolinic acid (54.02 mg, 56.94 μmol, 38.61% yield) as a white solid.

MS (ESI) m/z: 961.6 [M+H]⁺

¹H NMR (400 MHz, DMSO-d₆) δ=13.07-12.69 (m, 1H), 10.54 (s, 1H), 10.22-9.76 (m, 1H), 8.13 (s, 1H), 8.08-7.98 (m, 2H), 7.79 (d, J=8.0 Hz, 1H), 7.65-7.54 (m, 2H), 7.51-7.43 (m, 3H), 7.40-7.32 (m, 2H), 7.18 (d, J=9.2 Hz, 1H), 7.13-7.05 (m, 1H), 6.97 (d, J=8.4 Hz, 1H), 6.88 (d, J=8.4 Hz, 1H), 6.63 (d, J=7.6 Hz, 1H), 4.98 (s, 2H), 3.96 (t, J=6.0 Hz, 2H), 3.94-3.88 (m, 7H), 3.11-3.06 (m, 1H), 3.03 (t, J=5.6 Hz, 4H), 2.75 (t, J=6.8 Hz, 2H), 2.40-2.20 (m, 2H), 1.90 (s, 3H), 1.81-1.69 (m, 4H), 1.45-1.30 (m, 5H), 1.17 (t, J=7.2 Hz, 1H)

### Example 243. Preparation of Compound 310a

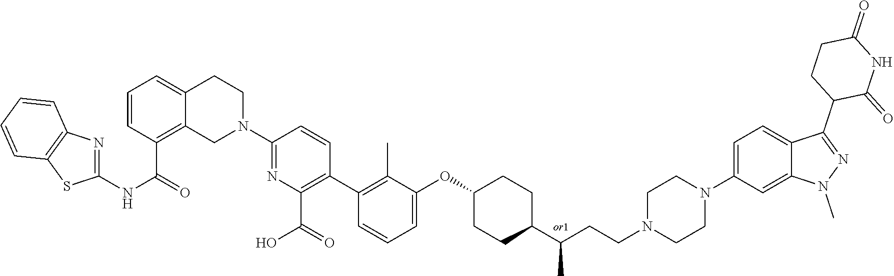 [SMILES: Cc1c(O[C@H]2CC[C@H]([C@H](C)CCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1R,4r)-4-((2R)-4-(4-(3-(2,6-dioxopiperidin-3-yl)-1-methyl-1H-indazol-6-yl)piperazin-1-yl)butan-2-yl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1R,4r)-4-((2R*)-4-(4-(3-(2,6-dioxopiperidin-3-yl)-1-methyl-1H-indazol-6-yl)piperazin-1-yl)butan-2-yl)cyclohexyl)oxy)-2-methylphenyl)picolinic acid]

### Step A. Procedure for Preparation of methyl (1r,4r)-4-(3-bromo-2-methylphenoxy)cyclohexane-1-carboxylate

To a solution of methyl (1s,4s)-4-hydroxycyclohexane-1-carboxylate (5 g, 31.61 mmol, 1 equiv.) and 3-bromo-2-methyl-phenol (7.09 g, 37.93 mmol, 1.2 equiv.) in toluene (50 mL) was added 2-(tributyl-phosphanylidene)acetonitrile (9.15 g, 37.93 mmol, 1.2 equiv.). The mixture was stirred at 110° C. for 16 hours under N₂ atmosphere. The reaction mixture was concentrated under reduced pressure to give a residue. The residue was purified by reverse-phase HPLC (with 0.1% formic acid) to give methyl (1r,4r)-4-(3-bromo-2-methylphenoxy)cyclohexane-1-carboxylate (4 g, 12.22 mmol, 19.34% yield) as a yellow oil.

¹H NMR (400 MHz, CDCl₃) δ=7.14 (d, J=8.0 Hz, 1H), 6.97 (t, J=8.0 Hz, 1H), 6.80 (d, J=8.4 Hz, 1H), 4.22-4.11 (m, 1H), 3.69 (s, 3H), 2.43-2.34 (m, 1H), 2.30 (s, 3H), 2.19-2.13 (m, 2H), 2.10-2.03 (m, 2H), 1.62-1.50 (m, 4H)

### Step B. Procedure for Preparation of (1r,4r)-4-(3-bromo-2-methylphenoxy)cyclohexane-1-carboxylic acid

To a solution of methyl (1r,4r)-4-(3-bromo-2-methylphenoxy)cyclohexane-1-carboxylate (3.95 g, 12.07 mmol, 1 equiv.) in THE (100 mL) and H₂O (10 mL) was added LiOH·H₂O (1.52 g, 36.21 mmol, 3 equiv.). The mixture was stirred at 25° C. for 16 hours. The reaction mixture was concentrated under reduced pressure to give a residue. The pH of the residue was adjusted to ˜3 by progressively adding diluted 1M HCl. The reaction mixture was partitioned between DCM (20 mL) and H₂O (5 mL). The organic phase was separated, dried over Na₂SO₄, and concentrated under reduced pressure to give (1r,4r)-4-(3-bromo-2-methylphenoxy)cyclohexane-1-carboxylic acid (3.6 g, 10.84 mmol, 44.92% yield) as a yellow solid.

MS (ESI) m/z: 314.1 [M+H]⁺.

¹H NMR (400 MHz, CDCl₃) δ=7.15 (d, J=8.0 Hz, 1H), 6.98 (t, J=8.0 Hz, 1H), 6.80 (d, J=8.4 Hz, 1H), 4.21-4.15 (m, 1H), 2.48-2.38 (m, 1H), 2.30 (s, 3H), 2.19-2.08 (m, 4H), 1.65-1.52 (m, 4H)

### Step C. Procedure for Preparation of (1r,4r)-4-(3-bromo-2-methylphenoxy)-N-methoxy-N-methylcyclohexane-1-carboxamide

To a solution of (1r,4r)-4-(3-bromo-2-methylphenoxy)cyclohexane-1-carboxylic acid (3.4 g, 10.86 mmol, 1 equiv.) and N-methoxymethanamine (3.18 g, 32.57 mmol, 3 equiv. HCl) in DCM (5 mL) was added HATU (6.19 g, 16.28 mmol, 1.5 equiv.) and TEA (5.49 g, 54.28 mmol, 7.56 mL, 5 equiv.). The mixture was stirred at 25° C. for 2 hours. The reaction mixture was concentrated under reduced pressure to give a residue. The residue was purified by flash silica gel chromatography (0˜10% ethyl acetate/petroleum ether) to give (1r,4r)-4-(3-bromo-2-methylphenoxy)-N-methoxy-N-methylcyclohexane-1-carboxamide (3.3 g, 8.69 mmol, 80.04% yield, 93.8% purity) as a yellow solid.

MS (ESI) m/z: 356.1 [M+H]⁺.

¹H NMR (400 MHz, CDCL₃) δ=7.15 (d, J=8.0 Hz, 1H), 6.99 (t, J=8.0 Hz, 1H), 6.82 (d, J=8.0 Hz, 1H), 4.17 (d, J=11.6 Hz, 1H), 3.73 (s, 3H), 3.20 (s, 3H), 2.80-2.70 (m, 1H), 2.30 (s, 3H), 2.24 (dd, J=2.8, 12.4 Hz, 2H), 1.96-1.89 (m, 2H), 1.72-1.62 (m, 2H), 1.57-1.46 (m, 2H)

### Step D. Procedure for Preparation of 1-((1r,4r)-4-(3-bromo-2-methylphenoxy)cyclohexyl)ethan-1-one

A mixture of (1r,4r)-4-(3-bromo-2-methylphenoxy)-N-methoxy-N-methylcyclohexane-1-carboxamide (3.1 g, 8.70 mmol, 1 equiv.) in THE (30 mL) was degassed and purged with N₂ three times. To this mixture was slowly added MeMgBr (3 M, 5.80 mL, 2 equiv.). The reaction mixture was stirred at −78° C. for 0.5 hours under N₂ atmosphere. The mixture was slowly warmed to 25° C. and stirred for 15.5 hours under N₂ atmosphere. The reaction mixture was quenched by the addition of saturated NH₄Cl (10 mL) and extracted with DCM (50 mL×2). The combined organic layers were washed with brine (5 mL×2), dried over Na₂SO₄, filtered, and concentrated under reduced pressure to give 1-((1r,4r)-4-(3-bromo-2-methylphenoxy)cyclohexyl)ethan-1-one (2.7 g, crude) as a yellow oil.

¹H NMR (400 MHz, DMSO-d₆) δ=7.16-7.13 (m, 1H), 7.10-7.02 (m, 2H), 4.27 (d, J=4.8, 9.2 Hz, 1H), 2.45-2.38 (m, 1H), 2.25-2.21 (m, 3H), 2.12 (s, 3H), 2.09-2.03 (m, 2H), 1.95-1.86 (m, 2H), 1.47-1.35 (m, 4H)

### Step E. Procedure for Preparation of ethyl (E)-3-((1r,4r)-4-(3-bromo-2-methylphenoxy)cyclohexyl)but-2-enoate

A mixture of ethyl 2-diethoxyphosphorylacetate (2.16 g, 9.64 mmol, 1.91 mL, 1.2 equiv.) in THE (9 mL) was degassed and purged with N₂ three times. To this mixture was slowly added LiHMDS (1 M, 12.05 mL, 1.5 equiv.). The reaction mixture was stirred at −78° C. for 0.5 hours under N₂ atmosphere. To the reaction mixture was added 1-((1r,4r)-4-(3-bromo-2-methylphenoxy)cyclohexyl)ethan-1-one (2.5 g, 8.03 mmol, 1 equiv.) in THE (20 mL). The mixture was slowly warm to −25° C. and stirred for 2.5 hours under N₂ atmosphere. The reaction mixture was quenched by the addition of saturated NH₄Cl (10 mL) and extracted with ethyl acetate (10 mL×2). The combined organic layers were washed with brine (5 mL×2), dried over Na₂SO₄, and concentrated under reduced pressure to give a residue. The residue was purified by flash silica gel chromatography (0˜8% ethyl acetate/petroleum ether) to give ethyl (E)-3-((1r,4r)-4-(3-bromo-2-methylphenoxy)cyclohexyl)but-2-enoate (1.7 g, 4.27 mmol, 53.11% yield) as a yellow oil.

MS (ESI) m/z: 383.1 [M+H]⁺.

¹H NMR (400 MHz, DMSO-d₆) δ=7.19-7.13 (m, 1H), 7.11-7.01 (m, 2H), 5.67 (s, 1H), 4.33-4.23 (m, 1H), 4.06 (q, J=7.2 Hz, 2H), 3.33-3.27 (m, 1H), 2.21 (s, 2H), 2.10 (s, 6H), 1.74 (d, J=6.8 Hz, 2H), 1.62-1.48 (m, 1H), 1.43 (t, J=10.0 Hz, 3H), 1.19 (t, J=7.2 Hz, 3H)

### Step F. Procedure for Preparation of ethyl 3-((1r,4r)-4-(3-bromo-2-methylphenoxy)cyclohexyl)butanoate

To a solution of ethyl (E)-3-((1r,4r)-4-(3-bromo-2-methylphenoxy)cyclohexyl)but-2-enoate (1.7 g, 4.46 mmol, 1 equiv.) in EtOH (30 mL) was added PtO₂ (126.55 mg, 445.84 μmol, 80% purity, 0.1 equiv.) under N₂. The suspension was degassed under vacuum and purged with H₂ several times. The mixture was stirred under H₂ (15 psi) at 25° C. for 2 hours. The reaction mixture was filtered and concentrated under reduced pressure to give ethyl 3-((1r,4r)-4-(3-bromo-2-methylphenoxy)cyclohexyl)butanoate (1.7 g, crude) as a yellow oil.

¹H NMR (400 MHz, DMSO-d₆) δ=7.16-7.11 (m, 1H), 7.10-6.98 (m, 2H), 4.26-4.14 (m, 1H), 4.06 (q, J=7.2 Hz, 2H), 2.36 (dd, J=5.2, 14.8 Hz, 1H), 2.29-2.15 (m, 3H), 2.11-2.02 (m, 3H), 1.87-1.75 (m, 1H), 1.68 (d, J=10.4 Hz, 2H), 1.38-1.26 (m, 2H), 1.26-1.06 (m, 6H), 0.89-0.80 (m, 3H)

### Step G. Procedure for Preparation of 3-((1r,4r)-4-(3-bromo-2-methylphenoxy)cyclohexyl)butan-1-ol

To a solution of ethyl 3-((1r,4r)-4-(3-bromo-2-methylphenoxy)cyclohexyl)butanoate (1.7 g, 4.43 mmol, 1 equiv.) in THE (17 mL) was added LiAlH₄ (168.33 mg, 4.43 mmol, 1 equiv.). The mixture was stirred at 0° C. for 2 hours. The reaction mixture was quenched with Na₂SO₄·10 H₂O (100 mg) and slowly warmed to 20° C. The mixture was filtered and washed with DCM (50 mL). Then the organic layers were concentrated under reduced pressure to give a residue. The residue was purified by prep-HPLC to give 3-((1r,4r)-4-(3-bromo-2-methylphenoxy)cyclohexyl)butan-1-ol (600 mg, 1.64 mmol, 36.91% yield) as a yellow oil.

### Step H. Procedure for Preparation of (R)-3-((1r,4R)-4-(3-bromo-2-methylphenoxy)cyclohexyl)butan-1-ol

3-((1r,4r)-4-(3-bromo-2-methylphenoxy)cyclohexyl)butan-1-ol (480 mg, 1.41 mmol, 1 equiv.) was separated by SFC to give (R)-3-((1r,4R)-4-(3-bromo-2-methylphenoxy)cyclohexyl)butan-1-ol (90 mg, 240.24 μmol, 17.08% yield) as a white solid.

¹H NMR (400 MHz, DMSO-d₆) δ=7.15-7.10 (m, 1H), 7.09-7.00 (m, 2H), 4.31 (t, J=5.2 Hz, 1H), 4.25-4.13 (m, 1H), 3.50-3.34 (m, 2H), 2.20 (s, 3H), 2.08 (d, J=11.6 Hz, 2H), 1.69-1.60 (m, 2H), 1.56-1.42 (m, 2H), 1.38-1.28 (m, 3H), 1.21-1.10 (m, 3H), 0.80 (d, J=6.8 Hz, 3H)

### Step I. Procedure for Preparation of (R)-3-((1r,4R)-4-(3-bromo-2-methylphenoxy)cyclohexyl)butanal

To a solution of (R)-3-((1r,4R)-4-(3-bromo-2-methylphenoxy)cyclohexyl)butan-1-ol (90 mg, 263.71 μmol, 1 equiv.) in DCM (2 mL) was added DMP (111.85 mg, 263.71 μmol, 81.70 μL, 1 equiv.). The mixture was stirred at 0° C. for 2 hours. The reaction mixture was concentrated under reduced pressure to give a residue. The residue was purified by prep-TLC (SiO₂, petroleum ether:ethyl acetate=6:1) to give (R)-3-((1r,4R)-4-(3-bromo-2-methylphenoxy)cyclohexyl)butanal (50 mg, 130.87 μmol, 49.63% yield) as a yellow oil.

¹H NMR (400 MHz, DMSO-d₆) δ=9.67 (s, 1H), 7.16-7.12 (m, 1H), 7.10-7.00 (m, 2H), 4.29-4.16 (m, 1H), 2.25-2.16 (m, 4H), 2.08 (d, J=10.8 Hz, 2H), 2.01-1.90 (m, 1H), 1.69 (d, J=12.0 Hz, 2H), 1.24-1.11 (m, 6H), 0.86 (d, J=6.4 Hz, 3H)

### Step J. Procedure for Preparation of 3-(6-(4-((R)-3-((1r,4R)-4-(3-bromo-2-methylphenoxy)cyclohexyl)butyl)piperazin-1-yl)-1-methyl-1H-indazol-3-yl)piperidine-2,6-dione

A solution of (R)-3-((1r,4R)-4-(3-bromo-2-methylphenoxy)cyclohexyl)butanal (40 mg, 117.90 μmol, 1 equiv.) and 3-(1-methyl-6-piperazin-1-yl-indazol-3-yl)piperidine-2,6-dione (42.46 mg, 129.69 μmol, 1.1 equiv.) in DCM (1 mL) at 25° C. was stirred at 25° C. for 15.5 hours. NaBH(OAc)₃ (74.96 mg, 353.70 μmol, 3 equiv.) was added to the reaction mixture, and the resulting mixture was stirred at 25° C. for 0.5 hours. The reaction mixture was concentrated under reduced pressure to give a residue. The residue was purified by prep-TLC (SiO₂, DCM:MeOH=10:1) to give 3-(6-(4-((R)-3-((1r,4R)-4-(3-bromo-2-methylphenoxy)cyclohexyl)butyl)piperazin-1-yl)-1-methyl-1H-indazol-3-yl)piperidine-2,6-dione (40 mg, 60.86 μmol, 51.62% yield) as a yellow oil.

MS (ESI) m/z: 652.3 [M+H]⁺.

### Step K. Procedure for Preparation of tert-butyl 6-(8-(benzo[d]thiazol-2-ylcarbamoyl)-3,4-dihydroisoquinolin-2(1H)-yl)-3-(3-(((1R,4r)-4-((2R)-4-(4-(3-(2,6-dioxopiperidin-3-yl)-1-methyl-1H-indazol-6-yl)piperazin-1-yl)butan-2-yl)cyclohexyl)oxy)-2-methylphenyl)picolinate

To a solution of 3-(6-(4-((R)-3-((1r,4R)-4-(3-bromo-2-methylphenoxy)cyclohexyl)butyl)piperazin-1-yl)-1-methyl-1H-indazol-3-yl)piperidine-2,6-dione (40 mg, 61.48 μmol, 1 equiv.) and tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (45.19 mg, 73.77 μmol, 1.2 equiv.) in dioxane (2 mL) and H₂O (0.1 mL) was added [2-(2-aminophenyl)phenyl]palladium(1⁺); bis(1-adamantyl)-butyl-phosphane; methanesulfonate (4.48 mg, 6.15 μmol, 0.1 equiv.) and KF (10.71 mg, 184.43 μmol, 4.32 μL, 3 equiv.). The mixture was stirred at 100° C. for 16 hours. The reaction mixture was concentrated under reduced pressure to give a residue. The residue was purified by flash silica gel chromatography (0˜10% dichloromethane: methanol) to give tert-butyl 6-(8-(benzo[d]thiazol-2-ylcarbamoyl)-3,4-dihydroisoquinolin-2(1H)-yl)-3-(3-(((1R,4r)-4-((2R)-4-(4-(3-(2,6-dioxopiperidin-3-yl)-1-methyl-1H-indazol-6-yl)piperazin-1-yl)butan-2-yl)cyclohexyl)oxy)-2-methylphenyl)picolinate (35 mg, 29.06 μmol, 47.27% yield) as a yellow solid.

MS (ESI) m/z: 529.2 [½M+H]⁺.

### Step L. Procedure for Preparation of 6-(8-(benzo[d]thiazol-2-ylcarbamoyl)-3,4-dihydroisoquinolin-2(1H)-yl)-3-(3-(((1R,4r)-4-((2R)-4-(4-(3-(2,6-dioxopiperidin-3-yl)-1-methyl-1H-indazol-6-yl)piperazin-1-yl)butan-2-yl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1R,4r)-4-((2R*)-4-(4-(3-(2,6-dioxopiperidin-3-yl)-1-methyl-1H-indazol-6-yl)piperazin-1-yl)butan-2-yl)cyclohexyl)oxy)-2-methylphenyl)picolinic acid]

A solution of tert-butyl 6-(8-(benzo[d]thiazol-2-ylcarbamoyl)-3,4-dihydroisoquinolin-2(1H)-yl)-3-(3-(((1R,4r)-4-((2R)-4-(4-(3-(2,6-dioxopiperidin-3-yl)-1-methyl-1H-indazol-6-yl)piperazin-1-yl)butan-2-yl)cyclohexyl)oxy)-2-methylphenyl)picolinate (35 mg, 33.13 μmol, 1 equiv.) in DCM (0.5 mL) and TFA (0.2 mL) was stirred at 25° C. for 16 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1R,4r)-4-((2R)-4-(4-(3-(2,6-dioxopiperidin-3-yl)-1-methyl-1H-indazol-6-yl)piperazin-1-yl)butan-2-yl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1R,4r)-4-((2R*)-4-(4-(3-(2, 6-dioxopiperidin-3-yl)-1-methyl-1H-indazol-6-yl)piperazin-1-yl)butan-2-yl)cyclohexyl)oxy)-2-methylphenyl)picolinic acid] (20.05 mg, 19.22 μmol, 58.02% yield) as a yellow solid.

MS (ESI) m/z: 501.0 [M/2+H]⁺.

¹H NMR (400 MHz, DMSO-d₆) δ=12.97-12.74 (m, 1H), 10.87 (s, 1H), 8.03 (d, J=7.6 Hz, 1H), 7.79 (d, J=8.0 Hz, 1H), 7.62 (d, J=7.2 Hz, 1H), 7.57 (d, J=8.8 Hz, 1H), 7.51-7.43 (m, 3H), 7.40-7.32 (m, 2H), 7.12-7.05 (m, 1H), 7.02-6.91 (m, 4H), 6.62 (d, J=7.6 Hz, 1H), 4.98 (s, 2H), 4.28 (dd, J=5.2, 9.6 Hz, 1H), 4.22-4.15 (m, 2H), 3.97 (s, 4H), 3.92 (s, 5H), 3.64 (d, J=11.2 Hz, 2H), 3.09-2.99 (m, 4H), 2.69-2.60 (m, 2H), 2.39-2.21 (m, 2H), 2.21-2.10 (m, 3H), 1.87 (s, 3H), 1.79-1.67 (m, 3H), 1.58-1.48 (m, 1H), 1.46-1.32 (m, 3H), 1.31-1.18 (m, 3H), 0.88 (d, J=6.4 Hz, 3H)

### Example 244. Preparation of Compound 310b

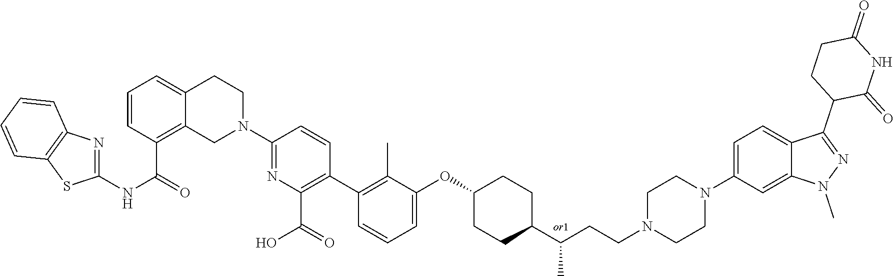 [SMILES: Cc1c(O[C@H]2CC[C@H]([C@@H](C)CCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1S,4r)-4-((2S)-4-(4-(3-(2,6-dioxopiperidin-3-yl)-1-methyl-1H-indazol-6-yl)piperazin-1-yl)butan-2-yl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1S,4r)-4-((2S*)-4-(4-(3-(2,6-dioxopiperidin-3-yl)-1-methyl-1H-indazol-6-yl)piperazin-1-yl)butan-2-yl)cyclohexyl)oxy)-2-methylphenyl)picolinic acid]

### Step A. Procedure for Preparation of (S)-3-((1r,4s)-4-(3-bromo-2-methylphenoxy)cyclohexyl)butanal

To a solution of (S)-3-((1r,4s)-4-(3-bromo-2-methylphenoxy)cyclohexyl)butan-1-ol (110 mg, 322.31 μmol, 1 equiv.) in DCM (1 mL) was added DMP (150.38 mg, 354.55 μmol, 109.84 μL, 1.1 equiv.) at 0° C. The mixture was stirred at 0° C. for 1 hour. The reaction mixture was concentrated under reduced pressure to give a residue. The residue was purified by prep-TLC (SiO₂, petroleum ether: ethyl acetate=6:1) to give (S)-3-((1r,4s)-4-(3-bromo-2-methylphenoxy)cyclohexyl)butanal (80 mg, 211.73 μmol, 65.69% yield) as a yellow oil.

### Step B. Procedure for Preparation of 3-(6-(4-((S)-3-((1r,4s)-4-(3-bromo-2-methylphenoxy)cyclohexyl)butyl)piperazin-1-yl)-1-methyl-1H-indazol-3-yl)piperidine-2,6-dione

To a solution of (S)-3-((1r,4s)-4-(3-bromo-2-methylphenoxy)cyclohexyl)butanal (70 mg, 206.33 mol, 1.0 equiv.) in DCM (1 mL) was added 3-(1-methyl-6-piperazin-1-yl-indazol-3-yl) piperidine-2, 6-dione (97.59 mg, 268.23 μmol, 1.3 equiv., HCl) at 25° C. The mixture was stirred at 25° C. for 16 hours. NaBH(OAc)₃ (174.92 mg, 825.31 μmol, 4.0 equiv.) was then added to the mixture at 25° C. The reaction mixture was stirred at 25° C. for 2 hours. The reaction mixture was filtered and concentrated under reduced pressure to give a residue. The residue was purified by prep-TLC (SiO₂, DCM:MeOH=20:1) to give 3-(6-(4-((S)-3-((1r,4s)-4-(3-bromo-2-methylphenoxy)cyclohexyl)butyl)piperazin-1-yl)-1-methyl-1H-indazol-3-yl)piperidine-2,6-dione (100 mg, 153.69 μmol, 74.49% yield) as a yellow oil.

MS (ESI) m/z: 650.3 [M+H]⁺.

### Step C. Procedure for Preparation of tert-butyl 6-(8-(benzo[d]thiazol-2-ylcarbamoyl)-3,4-dihydroisoquinolin-2(1H)-yl)-3-(3-(((1s,4r)-4-((2S)-4-(4-(3-(2,6-dioxopiperidin-3-yl)-1-methyl-1H-indazol-6-yl)piperazin-1-yl)butan-2-yl)cyclohexyl)oxy)-2-methylphenyl)picolinate

A mixture of tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl) pyridine-2-carboxylate (101.68 mg, 165.99 μmol, 1.2 equiv.), 3-(6-(4-((S)-3-((1r,4s)-4-(3-bromo-2-methylphenoxy)cyclohexyl)butyl)piperazin-1-yl)-1-methyl-1H-indazol-3-yl)piperidine-2,6-dione (90 mg, 138.32 μmol, 1.0 equiv.), KF (24.11 mg, 414.97 μmol, 9.72 μL, 3.0 equiv.), and [2-(2-aminophenyl) phenyl]palladium (1⁺); bis (1-adamantyl)-butyl-phosphane; methanesulfonate (20.15 mg, 27.66 μmol, 0.2 equiv.) in dioxane (1 mL) and H₂O (0.1 mL) was degassed and purged with N₂ three times. The mixture was stirred at 100° C. for 16 hours under a N₂ atmosphere. The reaction mixture was filtered and concentrated under reduced pressure to give a residue. The residue was purified by flash silica gel chromatography (0˜10% DCM/MeOH) to give tert-butyl 6-(8-(benzo[d]thiazol-2-ylcarbamoyl)-3,4-dihydroisoquinolin-2(1H)-yl)-3-(3-(((1s,4r)-4-((2S)-4-(4-(3-(2,6-dioxopiperidin-3-yl)-1-methyl-1H-indazol-6-yl)piperazin-1-yl)butan-2-yl)cyclohexyl)oxy)-2-methylphenyl)picolinate (120 mg, 96.33 μmol, 69.64% yield) as a yellow solid.

MS (ESI) m/z: 1057.7 [M+H]⁺.

### Step D. Procedure for Preparation of 6-(8-(benzo[d]thiazol-2-ylcarbamoyl)-3,4-dihydroisoquinolin-2(1H)-yl)-3-(3-(((1S,4r)-4-((2S)-4-(4-(3-(2,6-dioxopiperidin-3-yl)-1-methyl-1H-indazol-6-yl)piperazin-1-yl)butan-2-yl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1S,4r)-4-((2S*)-4-(4-(3-(2,6-dioxopiperidin-3-yl)-1-methyl-1H-indazol-6-yl)piperazin-1-yl)butan-2-yl)cyclohexyl)oxy)-2-methylphenyl)picolinic acid]

A solution of tert-butyl 6-(8-(benzo[d]thiazol-2-ylcarbamoyl)-3,4-dihydroisoquinolin-2(1H)-yl)-3-(3-(((1s,4r)-4-((2S)-4-(4-(3-(2,6-dioxopiperidin-3-yl)-1-methyl-1H-indazol-6-yl)piperazin-1-yl)butan-2-yl)cyclohexyl)oxy)-2-methylphenyl)picolinate (110 mg, 104.14 μmol, 1.0 equiv.) in TFA (0.5 mL) and DCM (0.5 mL) was stirred at 25° C. for 16 hours. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1S,4r)-4-((2S)-4-(4-(3-(2,6-dioxopiperidin-3-yl)-1-methyl-1H-indazol-6-yl)piperazin-1-yl)butan-2-yl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1S,4r)-4-((2S*)-4-(4-(3-(2,6-dioxopiperidin-3-yl)-1-methyl-1H-indazol-6-yl)piperazin-1-yl)butan-2-yl)cyclohexyl)oxy)-2-methylphenyl)picolinic acid] (24.29 mg, 22.70 μmol, 21.80% yield) as a white solid.

MS (ESI) m/z: 501.1 [M/2+H]⁺.

¹H NMR (400 MHz, DMSO-d₆) δ=10.85 (s, 1H), 8.03 (d, J=7.6 Hz, 1H), 7.79 (d, J=8.0 Hz, 1H), 7.62 (d, J=7.2 Hz, 1H), 7.52-7.42 (m, 4H), 7.40-7.32 (m, 2H), 7.11-7.04 (m, 1H), 6.98-6.89 (m, 3H), 6.84 (s, 1H), 6.61 (d, J=7.2 Hz, 1H), 4.98 (s, 2H), 4.29-4.13 (m, 2H), 3.95-3.85 (m, 5H), 3.22 (s, 2H), 3.02 (t, J=5.6 Hz, 2H), 2.70-2.55 (m, 5H), 2.42-2.23 (m, 4H), 2.17-2.07 (m, 3H), 1.87 (s, 3H), 1.74-1.65 (m, 2H), 1.63-1.55 (m, 1H), 1.48-1.14 (m, 8H), 0.86 (d, J=6.8 Hz, 3H)

### Example 245. Preparation of Compound 321a

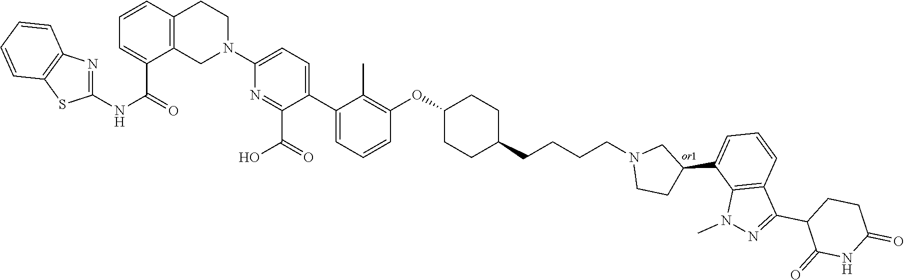 [SMILES: Cc1c(O[C@H]2CC[C@H](CCCCN3CC[C@H](c4cccc5c(C6CCC(=O)NC6=O)nn(C)c45)C3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(4-((3R)-3-(3-(2,6-dioxopiperidin-3-yl)-1-methyl-1H-indazol-7-yl)pyrrolidin-1-yl)butyl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1S,4r)-4-(4-((3R*)-3-(3-(2,6-dioxopiperidin-3-yl)-1-methyl-1H-indazol-7-yl)pyrrolidin-1-yl)butyl)cyclohexyl)oxy)-2-methylphenyl)picolinic acid]

### Step A. Procedure for Preparation of 4-((1r, 4s)-4-(3-bromo-2-methylphenoxy)cyclohexyl)butanal

A solution of DMSO (274.73 mg, 3.52 mmol, 274.73 μL, 4 equiv.) in DCM (20 mL) was added dropwise to a solution of (COCl)₂ (223.15 mg, 1.76 mmol, 153.89 μL, 2 equiv.) in DCM (20 mL) at −70° C. under N₂ atmosphere. The mixture was stirred at −70° C. for 1 hour. After which time, 4-((1r, 4s)-4-(3-bromo-2-methylphenoxy)cyclohexyl)butan-1-ol (300 mg, 879.04 μmol, 1 equiv.) in DCM (20 mL) was added dropwise at −70° C. The solution was stirred for 1 hour at −70° C. Then TEA (533.69 mg, 5.27 mmol, 734.10 μL, 6 equiv.) was added into the solution. The solution was stirred at −70° C. for 1 hour under N₂ atmosphere. The mixture was diluted with H₂O (10 mL) and extracted with DCM (10 mL×3). The combined organic layers were filtered and concentrated under reduced pressure to give 4-((1r, 4s)-4-(3-bromo-2-methylphenoxy)cyclohexyl)butanal (200 mg, crude) as a white solid.

### Step B. Procedure for Preparation of 3-(7-((R)-1-(4-((1r, 4s)-4-(3-bromo-2-methylphenoxy)cyclohexyl)butyl)pyrrolidin-3-yl)-1-methyl-1H-indazol-3-yl)piperidine-2,6-dione

To a solution of 4-((1r, 4s)-4-(3-bromo-2-methylphenoxy)cyclohexyl)butanal (150 mg, 442.13 mol, 1 equiv.) and 3-(1-methyl-7-((R)-pyrrolidin-3-yl)-1H-indazol-3-yl)piperidine-2,6-dione (165.73 mg, 530.56 μmol, 1.2 equiv.), NaOAc (36.27 mg, 442.13 μmol, 1 equiv.) in DCM (2 mL), was added NaBH(OAc)₃ (281.12 mg, 1.33 mmol, 3 equiv.). The mixture and stirred at 25° C. for 2.5 hours. The reaction mixture was filtered and concentrated under reduced pressure to give a residue. The residue was purified by column chromatography by prep-TLC (SiO₂, DCM:MeOH=10:1) to give 3-(7-((R)-1-(4-((1r, 4s)-4-(3-bromo-2-methylphenoxy)cyclohexyl)butyl)pyrrolidin-3-yl)-1-methyl-1H-indazol-3-yl)piperidine-2,6-dione (80 mg, 125.86 μmol, 28.47% yield) as a white solid.

MS (ESI) m/z: 635.4 [M+H]⁺.

### Step C. Procedure for Preparation of tert-butyl 6-(8-(benzo[d]thiazol-2-ylcarbamoyl)-3,4-dihydroisoquinolin-2(1H)-yl)-3-(3-(((1S,4r)-4-(4-((3R)-3-(3-(2,6-dioxopiperidin-3-yl)-1-methyl-1H-indazol-7-yl)pyrrolidin-1-yl)butyl)cyclohexyl)oxy)-2-methylphenyl)picolinate

A mixture of 3-(7-((R)-1-(4-((1r, 4S)-4-(3-bromo-2-methylphenoxy)cyclohexyl)butyl)pyrrolidin-3-yl)-1-methyl-1H-indazol-3-yl)piperidine-2,6-dione (80.00 mg, 125.86 μmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (92.51 mg, 151.03 μmol, 1.2 equiv.), [2-(2-aminophenyl)phenyl]palladium(1⁺); bis(1-adamantyl)-butyl-phosphane; methanesulfonate (9.17 mg, 12.59 μmol, 0.1 equiv.), and KF (1.5 M, 251.72 μL, 3 equiv.) in dioxane (3 mL) was degassed and purged with N₂ three times. The mixture was stirred at 100° C. for 12 hours under N₂ atmosphere. The reaction mixture was filtered and concentrated under reduced pressure to give a residue. The residue was purified by flash silica gel chromatography (0˜10% methanol:ethyl acetate) to give tert-butyl 6-(8-(benzo[d]thiazol-2-ylcarbamoyl)-3,4-dihydroisoquinolin-2(1H)-yl)-3-(3-(((1S,4r)-4-(4-((3R)-3-(3-(2,6-dioxopiperidin-3-yl)-1-methyl-1H-indazol-7-yl)pyrrolidin-1-yl)butyl)cyclohexyl)oxy)-2-methylphenyl)picolinate (98 mg, 94.11 μmol, 74.78% yield) as a white solid.

MS (ESI) m/z: 521.7 [M/2+H]⁺.

### Step D. Procedure for Preparation of 6-(8-(benzo[d]thiazol-2-ylcarbamoyl)-3,4-dihydroisoquinolin-2(1H)-yl)-3-(3-(((1s,4r)-4-(4-((3R)-3-(3-(2,6-dioxopiperidin-3-yl)-1-methyl-1H-indazol-7-yl)pyrrolidin-1-yl)butyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1S,4r)-4-(4-((3R*)-3-(3-(2,6-dioxopiperidin-3-yl)pyrrolidin-1-yl)butyl)cyclohexyl)oxy)-2-methylphenyl)picolinic acid]

A solution of tert-butyl 6-(8-(benzo[d]thiazol-2-ylcarbamoyl)-3,4-dihydroisoquinolin-2(1H)-yl)-3-(3-(((1S,4r)-4-(4-((3R)-3-(3-(2,6-dioxopiperidin-3-yl)-1-methyl-1H-indazol-7-yl)pyrrolidin-1-yl)butyl)cyclohexyl)oxy)-2-methylphenyl)picolinate (90.00 mg, 86.43 μmol, 1 equiv.) in TFA (1 mL) and DCM (1 mL) was stirred at 25° C. for 1 hour. The mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-(4-((3R)-3-(3-(2,6-dioxopiperidin-3-yl)-1-methyl-1H-indazol-7-yl)pyrrolidin-1-yl)butyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1S,4r)-4-(4-((3R*)-3-(3-(2,6-dioxopiperidin-3-yl)-1-methyl-1H-indazol-7-yl)pyrrolidin-1-yl)butyl)cyclohexyl)oxy)-2-methylphenyl)picolinic acid] (43.68 mg, 42.41 μmol, 49.07% yield) as an off-white solid.

MS (ESI) m/z: 985.8 [M+H]⁺.

¹H NMR (400 MHz, DMSO-d₆) δ=12.86 (d, J=16.9 Hz, 1H), 10.89 (s, 1H), 8.14 (s, 1H), 8.01 (d, J=8.0 Hz, 1H), 7.78 (d, J=8.0 Hz, 1H), 7.62 (d, J=7.6 Hz, 1H), 7.58 (d, J=8.0 Hz, 1H), 7.49-7.42 (m, 4H), 7.36 (td, J=7.6, 11.5 Hz, 2H), 7.13-7.03 (m, 2H), 6.92 (dd, J=8.8, 16.0 Hz, 2H), 6.62 (d, J=7.2 Hz, 1H), 4.97 (s, 2H), 4.36 (dd, J=4.8, 10.0 Hz, 1H), 4.24-4.16 (m, 5H), 3.91 (t, J=5.8 Hz, 2H), 3.02 (t, J=5.2 Hz, 4H), 2.87-2.74 (m, 2H), 2.70-2.60 (m, 2H), 2.45-2.27 (m, 3H), 2.18-1.99 (m, 4H), 1.87 (s, 3H), 1.78 (d, J=12.4 Hz, 2H), 1.57-1.50 (m, 2H), 1.41-1.14 (m, 8H), 1.10-1.00 (m, 2H)

### Example 246. Preparation of Compound 321b

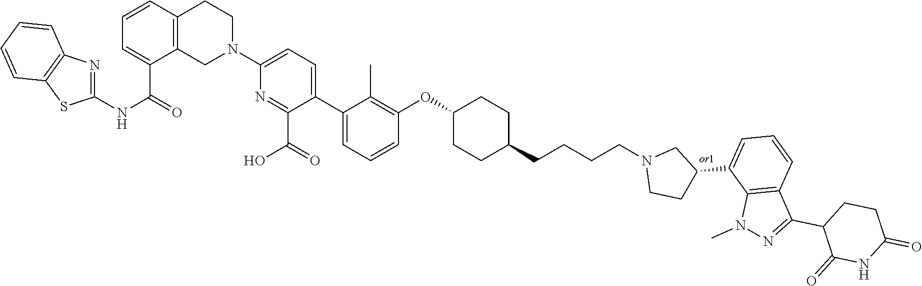 [SMILES: Cc1c(O[C@H]2CC[C@H](CCCCN3CC[C@@H](c4cccc5c(C6CCC(=O)NC6=O)nn(C)c45)C3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-(4-((3S)-3-(3-(2,6-dioxopiperidin-3-yl)-1-methyl-1H-indazol-7-yl)pyrrolidin-1-yl)butyl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1R,4r)-4-(4-((3S*)-3-(3-(2,6-dioxopiperidin-3-yl)-1-methyl-1H-indazol-7-yl)pyrrolidin-1-yl)butyl)cyclohexyl)oxy)-2-methylphenyl)picolinic acid]

### Step A. Procedure for Preparation of 4-((1r,4s)-4-(3-bromo-2-methylphenoxy)cyclohexyl)butanal

A mixture of DMSO (457.88 mg, 5.86 mmol, 457.88 μL, 4.0 equiv.) in DCM (50 mL) was degassed and purged with N₂ three times. To this solution was added oxalyl dichloride (371.91 mg, 2.93 mmol, 256.49 μL, 2.0 equiv.). The reaction mixture was stirred at −70° C. for 1 hour under N₂ atmosphere. A mixture of 4-((1r,4s)-4-(3-bromo-2-methylphenoxy)cyclohexyl)butan-1-ol (500 mg, 1.47 mmol, 1.0 equiv.) in DCM (100 mL) was added, and the resulting mixture was stirred at −70° C. for 1.5 hours under N₂ atmosphere. TEA (889.49 mg, 8.79 mmol, 1.22 mL, 6.0 equiv.) was added, and the mixture was stirred at −70° C. for 0.5 hour under N₂ atmosphere. The reaction mixture was diluted with H₂O (50 mL) and extracted with DCM (50 mL×2). The combined organic layers were concentrated under reduced pressure to give a residue. The residue was purified by flash silica gel chromatography (0˜20% ethyl acetate/petroleum ether) to give 4-((1r,4s)-4-(3-bromo-2-methylphenoxy)cyclohexyl)butanal (200 mg, 560.03 μmol, 38.23% yield) as a white solid.

¹H NMR (400 MHz, DMSO-d₆) δ=9.66 (s, 1H), 7.17-7.11 (m, 1H), 7.10-7.00 (m, 2H), 4.23 (t, J=10.1 Hz, 1H), 2.41 (t, J=6.8 Hz, 2H), 2.21 (s, 3H), 2.04 (d, J=10.8 Hz, 2H), 1.76 (d, J=12.4 Hz, 2H), 1.58-1.50 (m, 2H), 1.41-1.30 (m, 2H), 1.29-1.23 (m, 1H), 1.22-1.15 (m, 2H), 1.08-0.98 (m, 2H)

### Step B. Procedure for Preparation of 3-(7-((S)-1-(4-((1r,4r)-4-(3-bromo-2-methylphenoxy)cyclohexyl)butyl)pyrrolidin-3-yl)-1-methyl-1H-indazol-3-yl)piperidine-2,6-dione

To a solution of 4-((1r,4s)-4-(3-bromo-2-methylphenoxy)cyclohexyl)butanal (100 mg, 294.75 mol, 1.0 equiv.) in DCM (2 mL) was added 3-[1-methyl-7-[(3S)-pyrrolidin-3-yl]indazol-3-yl]piperidine-2,6-dione (110.49 mg, 353.70 μmol, 1.2 equiv.). The mixture was stirred at 25° C. for 1 hour. NaBH(OAc)₃ (187.41 mg, 884.26 μmol, 3.0 equiv.) was added to the mixture which was stirred for 0.5 hour. The reaction mixture was diluted with H₂O (5 mL) and extracted with DCM (5 mL×2). The combined organic layers were concentrated under reduced pressure to give 3-(7-((S)-1-(4-((1r,4r)-4-(3-bromo-2-methylphenoxy)cyclohexyl)butyl)pyrrolidin-3-yl)-1-methyl-1H-indazol-3-yl)piperidine-2,6-dione (200 mg, 289.48 μmol, 98.21% yield) as a white solid.

¹H NMR (400 MHz, DMSO-d₆) δ=10.88 (s, 1H), 7.61-7.52 (m, 1H), 7.47-7.41 (m, 1H), 7.16-7.00 (m, 4H), 4.35 (dd, J=5.6, 9.2 Hz, 1H), 4.22 (s, 3H), 3.20 (s, 1H), 2.68-2.61 (m, 4H), 2.33 (s, 1H), 2.21 (s, 3H), 2.17-2.12 (m, 1H), 2.07-2.02 (m, 2H), 1.91 (s, 5H), 1.78 (d, J=10.4 Hz, 2H), 1.58-1.49 (m, 2H), 1.43-1.17 (m, 10H), 1.09-0.98 (m, 2H)

MS (ESI) m/z: 635.5 [M+H]⁺.

### Step C. Procedure for Preparation of tert-butyl 6-(8-(benzo[d]thiazol-2-ylcarbamoyl)-3,4-dihydroisoquinolin-2(1H)-yl)-3-(3-(((1r,4r)-4-(4-((3S)-3-(3-(2,6-dioxopiperidin-3-yl)-1-methyl-1H-indazol-7-yl)pyrrolidin-1-yl)butyl)cyclohexyl)oxy)-2-methylphenyl)picolinate

To a solution of tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (106.00 mg, 173.06 mol, 1.1 equiv.), 3-(7-((S)-1-(4-((1r,4r)-4-(3-bromo-2-methylphenoxy)cyclohexyl)butyl)pyrrolidin-3-yl)-1-methyl-1H-indazol-3-yl)piperidine-2,6-dione (100 mg, 157.32 μmol, 1.0 equiv.), and KF (1.5 M, 314.65 μL, 3.0 equiv.) in dioxane (2 mL), was added [2-(2-aminophenyl)phenyl]palladium(1⁺); bis(1-adamantyl)-butyl-phosphane; methanesulfonate (22.91 mg, 31.46 μmol, 0.2 equiv.). The mixture was stirred and heated at 100° C. for 1.5 hours. The reaction mixture was concentrated under reduced pressure to give a residue. The residue was purified by flash silica gel chromatography (0˜10% DCM/MeOH gradient) to give tert-butyl 6-(8-(benzo[d]thiazol-2-ylcarbamoyl)-3,4-dihydroisoquinolin-2(1H)-yl)-3-(3-(((1r,4r)-4-(4-((3S)-3-(3-(2,6-dioxopiperidin-3-yl)-1-methyl-1H-indazol-7-yl)pyrrolidin-1-yl)butyl)cyclohexyl)oxy)-2-methylphenyl)picolinate (90 mg, crude) as a white solid.

MS (ESI) m/z: 521.7 [M+H]⁺.

### Step D. Procedure for Preparation of 6-(8-(benzo[d]thiazol-2-ylcarbamoyl)-3,4-dihydroisoquinolin-2(1H)-yl)-3-(3-(((1r,4r)-4-(4-((3S)-3-(3-(2,6-dioxopiperidin-3-yl)-1-methyl-1H-indazol-7-yl)pyrrolidin-1-yl)butyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1R,4r)-4-(4-((3S*)-3-(3-(2,6-dioxopiperidin-3-yl)-1-methyl-1H-indazol-7-yl)pyrrolidin-1-yl)butyl)cyclohexyl)oxy)-2-methylphenyl)picolinic acid]

To a solution of tert-butyl 6-(8-(benzo[d]thiazol-2-ylcarbamoyl)-3,4-dihydroisoquinolin-2(1H)-yl)-3-(3-(((1r,4r)-4-(4-((3S)-3-(3-(2,6-dioxopiperidin-3-yl)-1-methyl-1H-indazol-7-yl)pyrrolidin-1-yl)butyl)cyclohexyl)oxy)-2-methylphenyl)picolinate (90.00 mg, 86.43 μmol, 1.0 equiv.) in DCM (1 mL) was added TFA (1 mL). The mixture was stirred at 25° C. for 2 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1r,4r)-4-(4-((3S)-3-(3-(2,6-dioxopiperidin-3-yl)-1-methyl-1H-indazol-7-yl)pyrrolidin-1-yl)butyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1R,4r)-4-(4-((3 S*)-3-(3-(2,6-dioxopiperidin-3-yl)-1-methyl-1H-indazol-7-yl)pyrrolidin-1-yl)butyl)cyclohexyl)oxy)-2-methylphenyl)picolinic acid](39.67 mg, 39.86 μmol, 46.12% yield) as a white solid.

MS (ESI) m/z: 985.8 [M+H]⁺.

¹H NMR (400 MHz, DMSO-d₆) δ=10.89 (s, 1H), 8.03 (d, J=8.4 Hz, 1H), 7.79 (d, J=8.4 Hz, 1H), 7.61 (t, J=6.4 Hz, 2H), 7.50-7.41 (m, 4H), 7.40-7.31 (m, 2H), 7.13 (t, J=7.6 Hz, 1H), 7.10-7.03 (m, 1H), 6.99-6.88 (m, 2H), 6.61 (d, J=7.2 Hz, 1H), 4.97 (s, 2H), 4.40-4.33 (m, 1H), 4.29-4.14 (m, 5H), 3.94-3.88 (m, 2H), 3.08-2.97 (m, 4H), 2.61 (s, 3H), 2.32 (d, J=1.6 Hz, 2H), 2.22-2.03 (m, 5H), 1.87 (s, 3H), 1.78 (d, J=14.0 Hz, 2H), 1.63-1.54 (m, 2H), 1.45-1.30 (m, 5H), 1.29-1.21 (m, 3H), 1.17-0.97 (m, 3H)

### Example 247. Preparation of Compound 320a

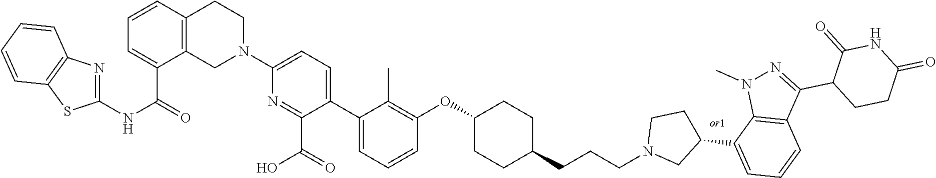 [SMILES: Cc1c(O[C@H]2CC[C@H](CCCN3CC[C@H](c4cccc5c(C6CCC(=O)NC6=O)nn(C)c45)C3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(3-((3R)-3-(3-(2,6-dioxopiperidin-3-yl)-1-methyl-1H-indazol-7-yl)pyrrolidin-1-yl)propyl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1S,4r)-4-(3-((3R*)-3-(3-(2,6-dioxopiperidin-3-yl)-1-methyl-1H-indazol-7-yl)pyrrolidin-1-yl)propyl)cyclohexyl)oxy)-2-methylphenyl)picolinic acid]

### Step A. Procedure for Preparation of tert-butyl (3R)-3-[3-(2,6-dioxo-3-piperidyl)-1-methyl-indazol-7-yl]pyrrolidine-1-carboxylate

A mixture of tert-butyl (3R)-3-[3-(2,6-dibenzyloxy-3-pyridyl)-1-methyl-indazol-7-yl]pyrrolidine-1-carboxylate (4.30 g, 7.28 mmol, 1 equiv.), Pd(OH)₂ (1 g, 7.12 mmol, 0.978 equiv.), AcOH (437.14 mg, 7.28 mmol, 416.72 μL, 1 equiv.), and Pd/C (1 g, 10% purity, 1.00 equiv.) in THF (25 mL) and EtOH (25 mL) was degassed and purged with H₂ three times. The mixture was stirred at 20° C. for 16 hours under H₂ (15 Psi) atmosphere. The reaction mixture was filtered and concentrated under reduced pressure to give tert-butyl (3R)-3-[3-(2,6-dioxo-3-piperidyl)-1-methyl-indazol-7-yl]pyrrolidine-1-carboxylate (2.7 g, 6.55 mmol, 89.92% yield) as a gray gum.

MS (ESI) m/z: 357.3 [M+H]⁺

### Step B. Procedure for Preparation of 3-[1-methyl-7-[(3R)-pyrrolidin-3-yl]indazol-3-yl]piperidine-2,6-dione

To a solution of tert-butyl (3R)-3-[3-(2,6-dioxo-3-piperidyl)-1-methyl-indazol-7-yl]pyrrolidine-1-carboxylate (2.7 g, 6.55 mmol, 1 equiv.) in DCM (20 mL) was added HCl/dioxane (4 M, 1.64 mL, 1 equiv.). The mixture was stirred at 20° C. for 1 hour. The reaction mixture was filtered and concentrated under reduced pressure to give a residue. The residue was triturated with MeCN at 20° C. for 10 min and filtered to give 3-[1-methyl-7-[(3R)-pyrrolidin-3-yl]indazol-3-yl]piperidine-2,6-dione (2.79 g, crude, HCl) as a yellow solid. MS (ESI) m/z: 313.1 [M+H]⁺. ¹H NMR (400 MHz, D₂O) δ=7.58 (d, J=8.0 Hz, 1H), 7.38 (d, J=7.2 Hz, 1H), 7.18 (t, J=7.6 Hz, 1H), 4.40 (dd, J=5.2, 11.6 Hz, 1H), 4.31 (q, J=8.0 Hz, 1H), 4.19 (s, 3H), 3.76 (dd, J=7.8, 11.6 Hz, 1H), 3.60-3.37 (m, 3H), 2.85-2.68 (m, 2H), 2.51-2.38 (m, 2H), 2.28-2.17 (m, 2H).

### Step C. Procedure for Preparation of 3-((1r,4r)-4-(3-bromo-2-methylphenoxy)cyclohexyl)propanal

A mixture of 3-((1r,4s)-4-(3-bromo-2-methylphenoxy)cyclohexyl)propan-1-ol (800 mg, 2.44 mmol, 1.0 equiv.) and DMP (2.07 g, 4.89 mmol, 1.51 mL, 2.0 equiv.) in DCM (10 mL) was degassed and purged with N₂ three times at 0° C. The mixture was stirred at 25° C. for 6 hours under N₂ atmosphere. The reaction mixture was filtered and concentrated under reduced pressure to give a residue. The residue was purified by flash silica gel chromatography (0˜56% ethyl acetate/petroleum ether) to give 3-((1r,4r)-4-(3-bromo-2-methylphenoxy)cyclohexyl)propanal (600 mg, 1.84 mmol, 75.46% yield) as a white solid.

### Step D. Procedure for Preparation of 3-(7-((R)-1-(3-((1r,4s)-4-(3-bromo-2-methylphenoxy)cyclohexyl)propyl)pyrrolidin-3-yl)-1-methyl-1H-indazol-3-yl)piperidine-2,6-dione

A solution of 3-((1r,4r)-4-(3-bromo-2-methylphenoxy)cyclohexyl)propanal (100 mg, 307.47 mol, 1.0 equiv.), 3-[1-methyl-7-[(3R)-pyrrolidin-3-yl]indazol-3-yl]piperidine-2,6-dione (105.65 mg, 338.21 μmol, 1.1 equiv.), NaOAc (37.83 mg, 461.20 μmol, 1.5 equiv.), and NaBH(OAc)₃ (325.82 mg, 1.54 mmol, 5.0 equiv.) in DCM (2 mL) was degassed and purged with N₂ three times. The mixture was stirred at 25° C. for 3 hours under N₂ atmosphere. The reaction mixture was filtered and concentrated under reduced pressure to give a residue. The residue was purified by flash silica gel chromatography (0˜8% MeOH/DCM) to give 3-(7-((R)-1-(3-((1r,4s)-4-(3-bromo-2-methylphenoxy)cyclohexyl)propyl)pyrrolidin-3-yl)-1-methyl-1H-indazol-3-yl)piperidine-2,6-dione (70 mg, 112.61 μmol, 36.63% yield) as a white solid.

MS (ESI) m/z: 621.2 [M+H]⁺

### Step E. Procedure for Preparation of tert-butyl 6-(8-(benzo[d]thiazol-2-ylcarbamoyl)-3,4-dihydroisoquinolin-2(1H)-yl)-3-(3-(((1S,4r)-4-(3-((3R)-3-(3-(2,6-dioxopiperidin-3-yl)-1-methyl-1H-indazol-7-yl)pyrrolidin-1-yl)propyl)cyclohexyl)oxy)-2-methylphenyl)picolinate

A mixture of 3-(7-((R)-1-(3-((1r,4s)-4-(3-bromo-2-methylphenoxy)cyclohexyl)propyl)pyrrolidin-3-yl)-1-methyl-1H-indazol-3-yl)piperidine-2,6-dione (70 mg, 112.61 μmol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (82.78 mg, 135.13 μmol, 1.2 equiv.), Ad₂nBuP Pd G₃ (cataCXium® A Pd G₃) (16.40 mg, 22.52 μmol, 0.2 equiv.), and KF (19.63 mg, 337.83 μmol, 7.91 μL, 3.0 equiv.) in dioxane (2 mL) and H₂O (0.2 mL) was degassed and purged with N₂ three times. The mixture was stirred at 80° C. for 12 hours under N₂ atmosphere. The reaction mixture was filtered and concentrated under reduced pressure to give a residue. The residue was purified by prep-TLC (SiO₂, DCM:MeOH=10:1) to give tert-butyl 6-(8-(benzo[d]thiazol-2-ylcarbamoyl)-3,4-dihydroisoquinolin-2(1H)-yl)-3-(3-(((1S,4r)-4-(3-((3R)-3-(3-(2,6-dioxopiperidin-3-yl)-1-methyl-1H-indazol-7-yl)pyrrolidin-1-yl)propyl)cyclohexyl)oxy)-2-methylphenyl)picolinate (60 mg, 58.41 μmol, 51.87% yield) as a yellow oil.

MS (ESI) m/z: 514.2 [½M+H]⁺

### Step F. Procedure for Preparation of 6-(8-(benzo[d]thiazol-2-ylcarbamoyl)-3,4-dihydroisoquinolin-2(1H)-yl)-3-(3-(((1s,4r)-4-(3-((3R)-3-(3-(2,6-dioxopiperidin-3-yl)-1-methyl-1H-indazol-7-yl)pyrrolidin-1-yl)propyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1S,4r)-4-(3-((3R*)-3-(3-(2,6-dioxopiperidin-3-yl)-1-methyl-1H-indazol-7-yl)pyrrolidin-1-yl)propyl)cyclohexyl)oxy)-2-methylphenyl)picolinic acid]

To a solution of tert-butyl 6-(8-(benzo[d]thiazol-2-ylcarbamoyl)-3,4-dihydroisoquinolin-2(1H)-yl)-3-(3-(((1S,4r)-4-(3-((3R)-3-(3-(2,6-dioxopiperidin-3-yl)-1-methyl-1H-indazol-7-yl)pyrrolidin-1-yl)propyl)cyclohexyl)oxy)-2-methylphenyl)picolinate (60 mg, 58.41 μmol, 1.0 equiv.) in DCM (1 mL) was added TFA (0.3 mL). The mixture was stirred at 40° C. for 15 hours. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-(3-((3R)-3-(3-(2,6-dioxopiperidin-3-yl)-1-methyl-1H-indazol-7-yl)pyrrolidin-1-yl)propyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1S,4r)-4-(3-((3R*)-3-(3-(2,6-dioxopiperidin-3-yl)-1-methyl-1H-indazol-7-yl)pyrrolidin-1-yl)propyl)cyclohexyl)oxy)-2-methylphenyl)picolinic acid] (15.57 mg, 15.54 μmol, 26.60% yield) as a white solid.

MS (ESI) m/z: 971.5 [M+H]⁺

¹H NMR (400 MHz, DMSO-d₆) δ=13.42-11.72 (m, 2H), 10.89 (s, 1H), 8.16 (s, 1H), 8.06-7.97 (m, 1H), 7.78 (d, J=8.0 Hz, 1H), 7.62 (d, J=6.4 Hz, 1H), 7.55 (d, J=8.0 Hz, 1H), 7.49-7.40 (m, 4H), 7.39-7.31 (m, 2H), 7.12-7.01 (m, 2H), 6.96-6.86 (m, 2H), 6.63 (d, J=6.4 Hz, 1H), 4.97 (s, 2H), 4.37-4.35 (m, 1H), 4.21 (s, 3H), 3.91-3.89 (m, 2H), 3.06-2.97 (m, 3H), 2.69-2.60 (m, 4H), 2.39-2.30 (m, 4H), 2.21-2.11 (m, 2H), 2.09-2.06 (m, 2H), 1.83-1.77 (m, 3H), 1.85-1.69 (m, 3H), 1.54 (d, J=7.6 Hz, 2H), 1.42-1.19 (m, 6H), 1.12-0.99 (m, 2H).

### Example 248. Preparation of Compound 320b

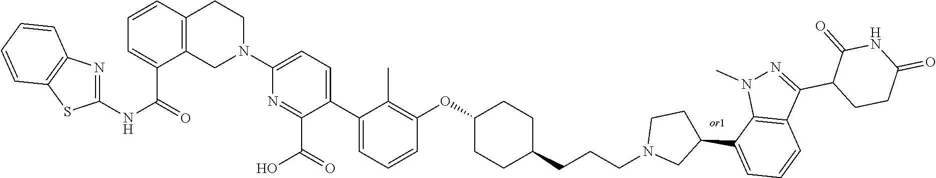 [SMILES: Cc1c(O[C@H]2CC[C@H](CCCN3CC[C@@H](c4cccc5c(C6CCC(=O)NC6=O)nn(C)c45)C3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-(3-((3S)-3-(3-(2,6-dioxopiperidin-3-yl)-1-methyl-1H-indazol-7-yl)pyrrolidin-1-yl)propyl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1R,4r)-4-(3-((3S*)-3-(3-(2,6-dioxopiperidin-3-yl)-1-methyl-1H-indazol-7-yl)pyrrolidin-1-yl)propyl)cyclohexyl)oxy)-2-methylphenyl)picolinic acid]

### Step A. Procedure for Preparation of tert-butyl 3-[3-(2,6-dibenzyloxy-3-pyridyl)-1-methyl-indazol-7-yl]pyrrolidine-1-carboxylate

A mixture of 7-bromo-3-(2,6-dibenzyloxy-3-pyridyl)-1-methyl-indazole (20 g, 39.97 mmol, 1.0 equiv.), tert-butyl 3-bromopyrrolidine-1-carboxylate (10.00 g, 39.97 mmol, 1.0 equiv.), Ir[dF(CF₃)ppy]₂(dtbpy)(PF₆) (448.42 mg, 399.69 μmol, 0.01 equiv.), NiCl₂.dtbbpy (795.38 mg, 2.00 mmol, 0.05 equiv.), quinuclidine (444.40 mg, 4.00 mmol, 0.1 equiv.), and TMP (11.29 g, 79.94 mmol, 13.57 mL, 2.0 equiv.) in MeCN (100 mL) was degassed three times. The reaction vial was then sealed with parafilm, placed 2 cm away from one blue LED, and irradiated at 25° C. for 14 hours. The mixture was filtered and concentrated under reduced pressure to give a residue. The residue was purified by reverse-phase HPLC to give tert-butyl 3-[3-(2,6-dibenzyloxy-3-pyridyl)-1-methyl-indazol-7-yl]pyrrolidine-1-carboxylate (8.73 g, 13.48 mmol, 33.73% yield) as a brown oil. MS (ESI) m/z: 591.4 [M+H]⁺. 1H NMR (400 MHz, DMSO-d₆) δ=8.14 (s, 1H), 7.84 (d, J=8.0 Hz, 1H), 7.61-7.13 (m, 12H), 7.00 (t, J=7.6 Hz, 1H), 6.59 (d, J=8.0 Hz, 1H), 5.43 (s, 4H), 4.30 (s, 3H), 4.25-4.17 (m, 1H), 3.75 (t, J=8.8 Hz, 1H), 3.49-3.39 (m, 3H), 2.33 (s, 1H), 2.12-2.04 (m, 1H), 1.43 (s, 9H)

### Step B. Procedure for Preparation of tert-butyl (3R)-3-[3-(2,6-dibenzyloxy-3-pyridyl)-1-methyl-indazol-7-yl]pyrrolidine-1-carboxylate and tert-butyl (3S)-3-[3-(2,6-dibenzyloxy-3-pyridyl)-1-methyl-indazol-7-yl]pyrrolidine-1-carboxylate

The stereoisomers of tert-butyl 3-[3-(2,6-dibenzyloxy-3-pyridyl)-1-methyl-indazol-7-yl]pyrrolidine-1-carboxylate (9.59 g, 16.23 mmol, 1 equiv.) were separated by prep-HPLC to afford tert-butyl (3S)-3-[3-(2,6-dibenzyloxy-3-pyridyl)-1-methyl-indazol-7-yl]pyrrolidine-1-carboxylate (4.5 g, 7.55 mmol, 46.51% yield) as a brown oil and tert-butyl (3R)-3-[3-(2,6-dibenzyloxy-3-pyridyl)-1-methyl-indazol-7-yl]pyrrolidine-1-carboxylate (4.3 g, 7.08 mmol, 43.63% yield) as a brown oil. ¹H NMR (400 MHz, DMSO-d₆) δ=7.84 (d, J=8.0 Hz, 1H), 7.53-7.20 (m, 12H), 7.00 (t, J=7.6 Hz, 1H), 6.59 (d, J=8.0 Hz, 1H), 5.43 (s, 4H), 4.30 (s, 3H), 4.25-4.16 (m, 1H), 3.80-3.69 (m, 1H), 3.50-3.38 (m, 3H), 2.33 (s, 1H), 2.07 (s, 1H), 1.43 (s, 9H).

¹H NMR (400 MHz, DMSO-d₆) δ=7.84 (d, J=8.0 Hz, 1H), 7.58-7.16 (m, 12H), 7.00 (t, J=7.6 Hz, 1H), 6.59 (d, J=8.0 Hz, 1H), 5.43 (s, 4H), 4.30 (s, 3H), 4.24-4.14 (m, 1H), 3.80-3.70 (m, 1H), 3.49-3.38 (m, 3H), 2.32 (s, 1H), 2.14-2.00 (m, 1H), 1.48-1.37 (m, 9H)

### Step C. Procedure for Preparation of tert-butyl (3S)-3-[3-(2,6-dioxo-3-piperidyl)-1-methyl-indazol-7-yl]pyrrolidine-1-carboxylate

To a solution of tert-butyl (3S)-3-[3-(2,6-dibenzyloxy-3-pyridyl)-1-methyl-indazol-7-yl]pyrrolidine-1-carboxylate (4.5 g, 7.62 mmol, 1 equiv.) and Pd(OH)₂ (937.50 mg, 6.68 mmol, 0.876 equiv.), Pd/C (937.50 mg, 880.94 μmol, 10% purity, 0.116 equiv.), and AcOH (457.47 mg, 7.62 mmol, 436.10 μL, 1 equiv.) in THE (25 mL) and EtOH (25 mL) was degassed and purged with H₂ three times. The mixture was stirred at 20° C. for 16 hours under H₂ atmosphere. The mixture was filtered and concentrated under reduced pressure to give tert-butyl (3S)-3-[3-(2,6-dioxo-3-piperidyl)-1-methyl-indazol-7-yl]pyrrolidine-1-carboxylate (3 g, 7.27 mmol, 95.47% yield) as a yellow gum. MS (ESI) m/z: 357.3 [M+H]⁺

### Step D. Procedure for Preparation of 3-[1-methyl-7-[(3S)-pyrrolidin-3-yl]indazol-3-yl]piperidine-2,6-dion

To a solution of tert-butyl (3S)-3-[3-(2,6-dioxo-3-piperidyl)-1-methyl-indazol-7-yl]pyrrolidine-1-carboxylate (3 g, 7.27 mmol, 1 eq) in DCM (20 mL) was added HCl/dioxane (4 M, 17.14 mL, 9.43 eq). The mixture was stirred at 20° C. for 1 hour. The reaction mixture was filtered and concentrated under reduced pressure to give a residue. The residue was triturated with MeCN (10 mL) at 20° C. for 10 min then filtered to give 3-[1-methyl-7-[(3S)-pyrrolidin-3-yl]indazol-3-yl]piperidine-2,6-dione (2.23 g, 6.72 mmol, 92.44% yield) as a white solid. MS (ESI) m/z: 313.0 [M+H]⁺. H NMR (400 MHz, D₂O) δ=7.61 (d, J=7.6 Hz, 1H), 7.40 (d, J=7.2 Hz, 1H), 7.20 (t, J=7.6 Hz, 1H), 4.43 (dd, J=5.2, 11.6 Hz, 1H), 4.38-4.31 (m, 1H), 4.21 (s, 3H), 3.78 (dd, J=7.6, 11.6 Hz, 1H), 3.63-3.53 (m, 1H), 3.52-3.38 (m, 2H), 2.86-2.69 (m, 2H), 2.58-2.38 (m, 2H), 2.33-2.16 (m, 2H).

### Step E. Procedure for Preparation of 3-(7-((S)-1-(3-((1r,4R)-4-(3-bromo-2-methylphenoxy)cyclohexyl)propyl)pyrrolidin-3-yl)-1-methyl-1H-indazol-3-yl)piperidine-2,6-dione

A mixture of 3-((1r,4r)-4-(3-bromo-2-methylphenoxy)cyclohexyl)propanal (100.00 mg, 307.47 mol, 1 equiv.), 3-(1-methyl-7-((S)-pyrrolidin-3-yl)-1H-indazol-3-yl)piperidine-2,6-dione (115.25 mg, 368.96 μmol, 1.2 equiv.), and NaBH(OAc)₃ (195.49 mg, 922.40 μmol, 3 equiv.) in DCM (4 mL) was degassed and purged with N₂ three times. The mixture was stirred at 25° C. for 10 hours under N₂ atmosphere. The reaction mixture was filtered and concentrated under reduced pressure to give a residue. The residue was purified by prep-TLC (SiO₂, DCM:MeOH=10:1) to give 3-(7-((S)-1-(3-((1r,4R)-4-(3-bromo-2-methylphenoxy)cyclohexyl)propyl)pyrrolidin-3-yl)-1-methyl-1H-indazol-3-yl)piperidine-2,6-dione (135 mg, 193.29 μmol, 62.87% yield) as a white oil.

MS (ESI) m/z: 623.4 [M+H]⁺

### Step F. Procedure for Preparation of tert-butyl 6-(8-(benzo[d]thiazol-2-ylcarbamoyl)-3,4-dihydroisoquinolin-2(1H)-yl)-3-(3-(((1r,4r)-4-(3-((3S)-3-(3-(2,6-dioxopiperidin-3-yl)-1-methyl-1H-indazol-7-yl)pyrrolidin-1-yl)propyl)cyclohexyl)oxy)-2-methylphenyl)picolinate

A mixture of 3-(7-((S)-1-(3-((1r,4R)-4-(3-bromo-2-methylphenoxy)cyclohexyl)propyl)pyrrolidin-3-yl)-1-methyl-1H-indazol-3-yl)piperidine-2,6-dione (135 mg, 217.18 μmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (172.94 mg, 282.33 μmol, 1.3 equiv.), Ad₂nBuP Pd G₃ (cataCXium® A Pd G₃) (31.63 mg, 43.44 μmol, 0.2 equiv.), and KF (1.5 M, 434.36 μL, 3 equiv.) in dioxane (5 mL) was degassed and purged with N₂ three times. The mixture was stirred at 80° C. for 10 hours under N₂ atmosphere. The reaction mixture was filtered and concentrated under reduced pressure to give a residue. The residue was purified by flash silica gel chromatography (0-9% ethyl acetate/methanol) to give compound tert-butyl 6-(8-(benzo[d]thiazol-2-ylcarbamoyl)-3,4-dihydroisoquinolin-2(1H)-yl)-3-(3-(((1r,4r)-4-(3-((3S)-3-(3-(2,6-dioxopiperidin-3-yl)-1-methyl-1H-indazol-7-yl)pyrrolidin-1-yl)propyl)cyclohexyl)oxy)-2-methylphenyl)picolinate (150 mg, 146.02 μmol, 67.23% yield) as a brown oil.

MS (ESI) m/z: 514.7 [M/2+H]⁺

### Step G. Procedure for Preparation of 6-(8-(benzo[d]thiazol-2-ylcarbamoyl)-3,4-dihydroisoquinolin-2(1H)-yl)-3-(3-(((1r,4r)-4-(3-((3S)-3-(3-(2,6-dioxopiperidin-3-yl)-1-methyl-1H-indazol-7-yl)pyrrolidin-1-yl)propyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1R,4r)-4-(3-((3S*)-3-(3-(2,6-dioxopiperidin-3-yl)-1-methyl-1H-indazol-7-yl)pyrrolidin-1-yl)propyl)cyclohexyl)oxy)-2-methylphenyl)picolinic acid]

A mixture of tert-butyl 6-(8-(benzo[d]thiazol-2-ylcarbamoyl)-3,4-dihydroisoquinolin-2(1H)-yl)-3-(3-(((1R,4r)-4-(3-((3S)-3-(3-(2,6-dioxopiperidin-3-yl)-1-methyl-1H-indazol-7-yl)pyrrolidin-1-yl)propyl)cyclohexyl)oxy)-2-methylphenyl)picolinate (140 mg, 136.28 μmol, 1 equiv.) in TFA (1.5 mL) and DCM (1.5 mL) was degassed and purged with N₂ three times. The mixture was stirred at 25° C. for 2 hours under N₂ atmosphere. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1r,4r)-4-(3-((3S)-3-(3-(2,6-dioxopiperidin-3-yl)-1-methyl-1H-indazol-7-yl)pyrrolidin-1-yl)propyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1R,4r)-4-(3-((3S*)-3-(3-(2,6-dioxopiperidin-3-yl)-1-methyl-1H-indazol-7-yl)pyrrolidin-1-yl)propyl)cyclohexyl)oxy)-2-methylphenyl)picolinic acid](78.67 mg, 79.21 μmol, 58.13% yield) as a white solid,

MS (ESI) m/z: 486.5 [M/2+H]⁺.

1H NMR (400 MHz, DMSO-d₆) δ=13.14-12.29 (m, 1H), 10.89 (s, 1H), 8.14 (s, 1H), 8.02 (d, J=8.0 Hz, 1H), 7.79 (d, J=8.4 Hz, 1H), 7.65-7.58 (m, 2H), 7.50-7.41 (m, 4H), 7.40-7.32 (m, 2H), 7.13 (t, J=7.6 Hz, 1H), 7.06 (t, J=8.0 Hz, 1H), 6.93 (dd, J=8.8, 15.6 Hz, 2H), 6.62 (d, J=7.6 Hz, 1H), 4.98 (s, 2H), 4.37 (dd, J=5.2, 10.0 Hz, 1H), 4.31-4.18 (m, 4H), 3.91 (r t, J=5.4 Hz, 2H), 3.62-3.46 (m, 2H), 3.21 (s, 2H), 3.01 (d, J=5.6 Hz, 4H), 2.69-2.61 (m, 2H), 2.43-2.29 (m, 2H), 2.19-2.06 (m, 4H), 1.87 (s, 3H), 1.80 (d, J=11.6 Hz, 2H), 1.63 (s, 2H), 1.40-1.24 (m, 5H), 1.14-1.01 (m, 2H)

### Example 249: Preparation of Compound 305

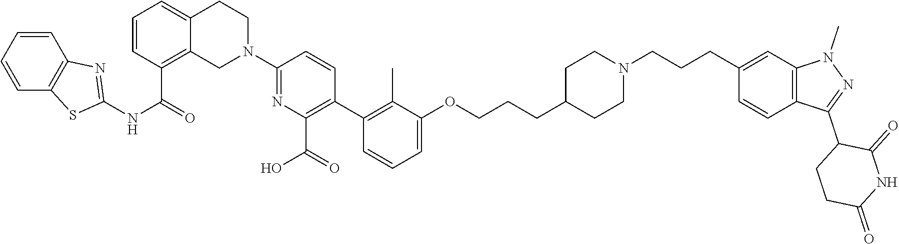 [SMILES: Cc1c(OCCCC2CCN(CCCc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-(1-(3-(3-(2,6-dioxopiperidin-3-yl)-1-methyl-1H-indazol-6-yl)propyl)piperidin-4-yl)propoxy)-2-methylphenyl)picolinic acid

### Step A. Procedure for Preparation of tert-butyl 4-(3-(3-bromo-2-methylphenoxy)propyl)piperidine-1-carboxylate

A mixture of 3-bromo-2-methylphenol (2 g, 10.69 mmol, 1 equiv.), tert-butyl 4-(3-hydroxypropyl) piperidine-1-carboxylate (3.12 g, 12.83 mmol, 1.2 equiv.), and 2-(tributyl-phosphanylidene) acetonitrile (5.16 g, 21.39 mmol, 2 equiv.) in toluene. (30 mL) was degassed and purged with N₂ three times. The mixture was stirred at 120° C. for 12 hours under N₂ atmosphere. The reaction mixture was filtered and concentrated under reduced pressure to give a residue. The residue was purified by flash silica gel chromatography (0˜7% ethyl acetate/petroleum ether) to give tert-butyl 4-(3-(3-bromo-2-methylphenoxy)propyl)piperidine-1-carboxylate (4.31 g, 10.45 mmol, 97.74% yield) as a white solid.

MS (ESI) m/z: 357.9 [M+H]⁺.

### Step B. Procedure for Preparation of tert-butyl 6-(8-(benzo[d]thiazol-2-ylcarbamoyl)-3,4-dihydroisoquinolin-2(1H)-yl)-3-(3-(3-(1-(tert-butoxycarbonyl)piperidin-4-yl)propoxy)-2-methylphenyl)picolinate

A mixture of tert-butyl 4-(3-(3-bromo-2-methylphenoxy)propyl)piperidine-1-carboxylate (500 mg, 1.21 mmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (891.28 mg, 1.46 mmol, 1.2 equiv.), Ad₂nBuP Pd G₃ (cataCXium® A Pd G₃) (264.92 mg, 363.76 μmol, 0.3 equiv.), and KF (105.67 mg, 1.82 mmol, 42.61 μL, 1.5 equiv.) in dioxane (2 mL) and H₂O (0.2 mL) was degassed and purged with N₂ three times. The mixture was stirred at 80° C. for 1 hour under N₂ atmosphere. The reaction was filtered and concentrated under reduced pressure to give a residue. The residue was purified by flash silica gel chromatography (0˜17% ethyl acetate/petroleum ether) to give tert-butyl 6-(8-(benzo[d]thiazol-2-ylcarbamoyl)-3,4-dihydroisoquinolin-2(1H)-yl)-3-(3-(3-(1-(tert-butoxycarbonyl)piperidin-4-yl)propoxy)-2-methylphenyl)picolinate (800 mg, 977.96 μmol, 80.65% yield) as a white solid.

MS (ESI) m/z: 818.9 [M+H]⁺.

### Step C. Procedure for Preparation of 6-(8-(benzo[d]thiazol-2-ylcarbamoyl)-3,4-dihydroisoquinolin-2(1H)-yl)-3-(2-methyl-3-(3-(piperidin-4-yl)propoxy)phenyl)picolinic acid

A solution of tert-butyl 6-(8-(benzo[d]thiazol-2-ylcarbamoyl)-3,4-dihydroisoquinolin-2(1H)-yl)-3-(3-(3-(1-(tert-butoxycarbonyl)piperidin-4-yl)propoxy)-2-methylphenyl)picolinate (800 mg, 977.96 μmol, 1 equiv.) in TFA (4 mL) and DCM (4 mL) was stirred at 25° C. for 2 hours. The reaction mixture was filtered and concentrated under reduced pressure to give 6-(8-(benzo[d]thiazol-2-ylcarbamoyl)-3,4-dihydroisoquinolin-2(1H)-yl)-3-(2-methyl-3-(3-(piperidin-4-yl)propoxy)phenyl)picolinic acid (460 mg, 902.68 μmol, 16.74% yield) as a yellow oil.

MS (ESI) m/z: 662.6 [M+H]⁺.

### Step D. Procedure for Preparation of 3-(2,6-bis(benzyloxy)pyridin-3-yl)-1-methyl-6-(3-((tetrahydro-2H-pyran-2-yl)oxy)prop-1-yn-1-yl)-1H-indazole

A mixture of 3-(2,6-bis(benzyloxy)pyridin-3-yl)-6-bromo-1-methyl-1H-indazole (3 g, 6.00 mmol, 1 equiv.), 2-(prop-2-yn-1-yloxy)tetrahydro-2H-pyran (840.43 mg, 6.00 mmol, 842.96 μL, 1 equiv.), CuI (228.36 mg, 1.20 mmol, 0.2 equiv.), Pd(PPh₃)₄ (692.80 mg, 599.54 μmol, 0.1 equiv.), and DMF (20 mL) in DIEA (20 mL) was degassed and purged with N₂ three times. The mixture was stirred at 80° C. for 16 hours under N₂ atmosphere. The reaction was filtered and concentrated under reduced pressure to give a residue. The residue was purified by flash silica gel chromatography (0˜13% ethyl acetate/petroleum ether) to give 3-(2,6-bis(benzyloxy)pyridin-3-yl)-1-methyl-6-(3-((tetrahydro-2H-pyran-2-yl)oxy)prop-1-yn-1-yl)-1H-indazole (1.2 g, 2.14 mmol, 35.76% yield) as a yellow oil.

MS (ESI) m/z: 560.3 [M+H]⁺.

### Step E. Procedure for Preparation of 3-(1-methyl-6-(3-((tetrahydro-2H-pyran-2-yl)oxy)propyl)-1H-indazol-3-yl)piperidine-2,6-dione

A mixture of 3-(2,6-bis(benzyloxy)pyridin-3-yl)-1-methyl-6-(3-((tetrahydro-2H-pyran-2-yl)oxy)prop-1-yn-1-yl)-1H-indazole (800 mg, 1.43 mmol, 1 equiv.), Pd/C (450 mg, 1.43 mmol, 10% purity, 1 equiv.), and Pd(OH)₂ (450 mg, 1.43 mmol, 30% purity, 1 equiv.) in THE (4 mL) was degassed and purged with H₂ three times. The mixture was stirred at 25° C. for 12 hours under H₂ atmosphere. The reaction mixture was filtered and concentrated under reduced pressure to give a residue. The residue was purified by prep-TLC (SiO₂, petroleum ether:ethyl acetate=1:1) to give 3-(1-methyl-6-(3-((tetrahydro-2H-pyran-2-yl)oxy)propyl)-1H-indazol-3-yl)piperidine-2,6-dione (300 mg, 778.30 μmol, 54.45% yield) as a white solid.

MS (ESI) m/z: 386.3 [M+H]⁺.

### Step F. Procedure for Preparation of 3-(6-(3-hydroxypropyl)-1-methyl-1H-indazol-3-yl)piperidine-2,6-dione

A mixture of 3-(1-methyl-6-(3-((tetrahydro-2H-pyran-2-yl)oxy)propyl)-1H-indazol-3-yl)piperidine-2,6-dione (300 mg, 778.30 μmol, 1 equiv.) in MeOH (4 mL) was added HCl (12 M, 12.97 μL, 0.2 equiv.) at 0° C. The mixture was stirred at 25° C. for 2 hours. The reaction mixture was filtered and concentrated under reduced pressure to give 3-(6-(3-hydroxypropyl)-1-methyl-1H-indazol-3-yl)piperidine-2,6-dione (340 mg, crude) as a yellow oil.

MS (ESI) m/z: 302.0 [M+H]⁺.

### Step G. Procedure for Preparation of 3-(3-(2,6-dioxopiperidin-3-yl)-1-methyl-1H-indazol-6-yl)propanal

A mixture of 3-(6-(3-hydroxypropyl)-1-methyl-1H-indazol-3-yl)piperidine-2,6-dione (220 mg, 730.07 μmol, 1 equiv.) and DMP (464.48 mg, 1.10 mmol, 339.29 μL, 1.5 equiv.) in DCM (4 mL) was stirred at 25° C. for 2 hours. The reaction mixture was filtered and concentrated under reduced pressure to give a residue. The residue was purified by flash silica gel chromatography (0˜20% ethyl acetate/petroleum ether) to give 3-(3-(2,6-dioxopiperidin-3-yl)-1-methyl-1H-indazol-6-yl)propanal (240 mg, 577.30 μmol, 79.07% yield) as a yellow solid.

MS (ESI) m/z: 300.1 [M+H]⁺.

### Step H. Procedure for Preparation of 6-(8-(benzo[d]thiazol-2-ylcarbamoyl)-3,4-dihydroisoquinolin-2(1H)-yl)-3-(3-(3-(1-(3-(3-(2,6-dioxopiperidin-3-yl)-1-methyl-1H-indazol-6-yl)propyl)piperidin-4-yl)propoxy)-2-methylphenyl)picolinic acid

A mixture of 6-(8-(benzo[d]thiazol-2-ylcarbamoyl)-3,4-dihydroisoquinolin-2(1H)-yl)-3-(2-methyl-3-(3-(piperidin-4-yl)propoxy)phenyl)picolinic acid (241.20 mg, 364.46 μmol, 1.0 equiv.), 3-(3-(2,6-dioxopiperidin-3-yl)-1-methyl-1H-indazol-6-yl)propanal (120 mg, 400.90 μmol, 1.1 equiv.), and 4A MS (30 mg) in DCM (6 mL) was stirred at 25° C. for 12 hours. To the reaction mixture was added NaBH(OAc)₃ (154.49 mg, 728.92 μmol, 2 equiv.). The resulting mixture was stirred at 25° C. for 0.5 hour. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(3-(1-(3-(3-(2,6-dioxopiperidin-3-yl)-1-methyl-1H-indazol-6-yl)propyl)piperidin-4-yl)propoxy)-2-methylphenyl)picolinic acid (89.66 mg, 89.27 μmol, 24.49% yield) as a yellow solid.

MS (ESI) m/z: 945.6 [M+H]⁺.

¹H NMR (400 MHz, DMSO-d₆) δ=10.88 (s, 1H), 8.15 (d, J=2.4 Hz, 1H), 8.01 (d, J=8.0 Hz, 1H), 7.78 (d, J=8.4 Hz, 1H), 7.62 (dd, J=4.0, 6.8 Hz, 2H), 7.46-7.39 (m, 4H), 7.39-7.30 (m, 2H), 7.07 (t, J=7.2 Hz, 1H), 6.99 (d, J=8.8 Hz, 1H), 6.94-6.90 (m, 1H), 6.86 (d, J=8.0 Hz, 1H), 6.63 (d, J=7.6 Hz, 1H), 5.01-4.94 (m, 2H), 4.33 (dd, J=5.2, 9.6 Hz, 1H), 3.95 (s, 5H), 3.91 (t, J=5.6 Hz, 2H), 3.12 (d, J=3.6 Hz, 2H), 3.02 (d, J=4.8 Hz, 2H), 2.72 (d, J=7.2 Hz, 3H), 2.69-2.61 (m, 5H), 2.20-2.14 (m, 1H), 1.89 (s, 5H), 1.73 (d, J=2.4 Hz, 4H), 1.48-1.30 (m, 4H), 1.25-1.14 (m, 2H)

### Example 250. Preparation of Compound 307a

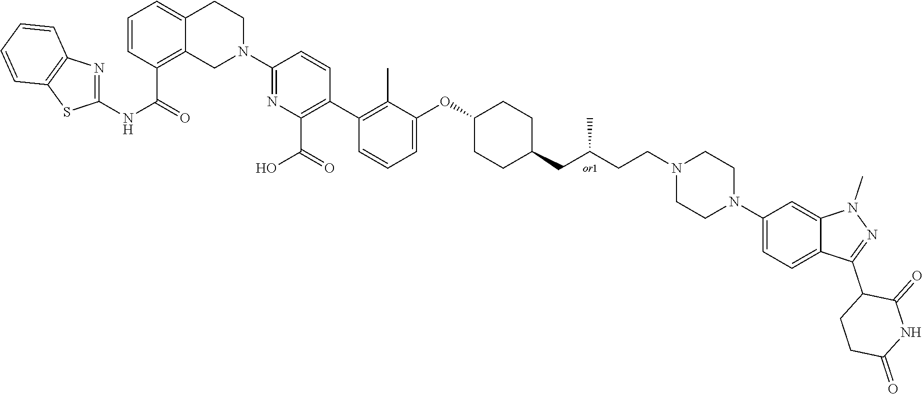 [SMILES: Cc1c(O[C@H]2CC[C@H](C[C@H](C)CCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-((2S)-4-(4-(3-(2,6-dioxopiperidin-3-yl)-1-methyl-1H-indazol-6-yl)piperazin-1-yl)-2-methylbutyl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1S,4r)-4-((2S*)-4-(4-(3-(2,6-dioxopiperidin-3-yl)-1-methyl-1H-indazol-6-yl)piperazin-1-yl)-2-methylbutyl)cyclohexyl)oxy)-2-methylphenyl)picolinic acid]

### Step A. Procedure for Preparation of methyl 2-(4-hydroxycyclohexyl)acetate

To a solution of 2-(4-hydroxycyclohexyl)acetic acid (10.0 g, 63.21 mmol, 1 equiv.) in MeOH (100 mL) was added H₂SO₄ (18.60 g, 189.64 mmol, 10.11 mL, 3.0 equiv.). The mixture was stirred at 70° C. for 3 hours. The reaction mixture was diluted with H₂O (100 mL) and extracted with DCM (150 mL×3). The combined organic layers were dried over Na₂SO₄, filtered, and concentrated under reduced pressure to give methyl 2-(4-hydroxycyclohexyl)acetate (10.0 g, crude) as a yellow oil.

### Step B. Procedure for Preparation of methyl 2-[4-(3-bromo-2-methyl-phenoxy)cyclohexyl]acetate

A mixture of methyl 2-(4-hydroxycyclohexyl)acetate (10 g, 58.06 mmol, 1.0 equiv.), 3-bromo-2-methyl-phenol (21.72 g, 116.13 mmol, 2.0 equiv.), and 2-(tributyl-phosphanylidene)acetonitrile (16.82 g, 69.68 mmol, 1.2 equiv.) in toluene (100 mL) was degassed and purged with N₂ three times, and then the mixture was stirred at 120° C. for 16 hours under a N₂ atmosphere. The mixture was concentrated under reduced pressure to give a residue. The residue was purified by flash silica gel chromatography (0˜4% ethyl acetate/petroleum ether) to give methyl 2-[4-(3-bromo-2-methyl-phenoxy)cyclohexyl]acetate (14.5 g, crude) as a yellow oil.

¹H NMR (400 MHz, DMSO-d₆) δ=7.16-7.11 (m, 1H), 7.06 (t, J=8.0 Hz, 1H), 6.82-6.78 (m, 1H), 4.63 (s, 1H), 3.60-3.57 (m, 3H), 2.26 (s, 3H), 2.22 (d, J=13.2 Hz, 2H), 1.93-1.74 (m, 3H), 1.62-1.48 (m, 3H), 1.46-1.22 (m, 3H)

### Step C. Procedure for Preparation of 2-[4-(3-bromo-2-methyl-phenoxy)cyclohexyl]acetic acid

To a solution of methyl 2-[4-(3-bromo-2-methyl-phenoxy)cyclohexyl]acetate (14.00 g, 41.03 mmol, 1.0 equiv.) in THE (100 mL) and H₂O (30 mL) was added LiOH·H₂O (5.16 g, 123.08 mmol, 3.0 equiv.). The mixture was stirred at 30° C. for 16 hours. The reaction mixture was concentrated under reduced pressure to remove THE (100 mL). The residue was diluted with H₂O (20 mL), and its pH was adjusted with HCl (1 M) to 4. The resulting mixture was extracted with EtOAc (100 mL×3). The combined organic layers were washed with brine (100 mL×1), dried over Na₂SO₄, filtered, and concentrated under reduced pressure to give a residue. The residue was purified by column chromatography (SiO₂, petroleum ether/ethyl acetate=50/1 to 10/1) to give 2-[4-(3-bromo-2-methyl-phenoxy)cyclohexyl]acetic acid (7.75 g, crude) as a yellow oil.

¹H NMR (400 MHz, DMSO-d₆) δ=12.03 (s, 1H), 7.14-7.11 (m, 1H), 7.07 (t, J=8.0 Hz, 1H), 7.00-6.96 (m, 1H), 4.64 (s, 1H), 2.26 (s, 3H), 2.15 (d, J=7.2 Hz, 2H), 1.92-1.84 (m, 2H), 1.83-1.71 (m, 2H), 1.55-1.50 (m, 2H), 1.43-1.25 (m, 3H)

### Step D. Procedure for Preparation of 2-[4-(3-bromo-2-methyl-phenoxy)cyclohexyl]-N-methoxy-N-methyl-acetamide

To a solution of 2-[4-(3-bromo-2-methyl-phenoxy)cyclohexyl]acetic acid (7.75 g, 23.68 mmol, 1.0 equiv.) in DCM (80 mL) was added HATU (10.81 g, 28.42 mmol, 1.2 equiv.), DIEA (15.31 g, 118.42 mmol, 20.63 mL, 5 equiv.) and N-methoxymethanamine (6.93 g, 71.05 mmol, 3.0 equiv., HCl). The mixture was stirred at 20° C. for 16 hours. The reaction mixture was concentrated under reduced pressure to give a residue. The residue was purified by flash silica gel chromatography (0˜5% ethyl acetate/petroleum ether) to give 2-[4-(3-bromo-2-methyl-phenoxy)cyclohexyl]-N-methoxy-N-methyl-acetamide (7.0 g, 18.38 mmol, 77.61% yield) as a yellow oil.

MS (ESI) m/z: 369.9 [M+H]⁺

### Step E. Procedure for Preparation of 2-((1r,4r)-4-(3-bromo-2-methylphenoxy)cyclohexyl)-N-methoxy-N-methylacetamide

Stereoisomers of 2-[4-(3-bromo-2-methyl-phenoxy)cyclohexyl]-N-methoxy-N-methyl-acetamide (7.0 g, 18.90 mmol, 1.0 equiv.) was separated by prep-HPLC to give 2-((1r,4r)-4-(3-bromo-2-methylphenoxy)cyclohexyl)-N-methoxy-N-methylacetamide (1.6 g, 4.23 mmol, 22.38% yield) as a yellow solid.

MS (ESI) m/z: 370.2 [M+H]⁺

¹H NMR (400 MHz, DMSO-d₆) δ=7.16-7.12 (m, 1H), 7.07 (t, J=8.0 Hz, 1H), 7.03-6.99 (m, 1H), 4.28-4.16 (m, 1H), 3.65 (s, 3H), 3.08 (s, 3H), 2.29 (d, J=6.4 Hz, 2H), 2.21 (s, 3H), 2.08-2.00 (m, 2H), 1.82-1.69 (m, 3H), 1.43-1.31 (m, 2H), 1.20-1.05 (m, 2H)

### Step F. Procedure for Preparation of 1-((1r,4r)-4-(3-bromo-2-methylphenoxy)cyclohexyl)propan-2-one

To a solution of 2-((1r,4r)-4-(3-bromo-2-methylphenoxy)cyclohexyl)-N-methoxy-N-methylacetamide (1.6 g, 4.32 mmol, 1.0 equiv.) in THE (16 mL) was added MeMgBr (3 M, 2.88 mL, 2 equiv.) dropwise at −70° C. under N₂ atmosphere. The mixture was stirred at 20° C. for 2 hours under N₂ atmosphere. The reaction mixture was added dropwise into saturated NH₄Cl solution 20 mL at 0° C. and extracted with EtOAc (30 mL×3). The combined organic layers were washed with brine (50 mL×1), dried over Na₂SO₄, filtered, and concentrated under reduced pressure to give 1-((1r,4r)-4-(3-bromo-2-methylphenoxy)cyclohexyl)propan-2-one (1.4 g, crude) as a yellow oil.

¹H NMR (400 MHz, DMSO-d₆) δ=7.17-7.11 (m, 1H), 7.06 (t, J=8.0 Hz, 1H), 7.03-6.98 (m, 1H), 4.27-4.16 (m, 1H), 2.34 (d, J=6.4 Hz, 2H), 2.21 (s, 3H), 2.07 (s, 3H), 2.04-1.98 (m, 2H), 1.82-1.66 (m, 3H), 1.44-1.31 (m, 2H), 1.12-1.00 (m, 2H)

### Step G. Procedure for Preparation of ethyl (E)-4-((1r,4r)-4-(3-bromo-2-methylphenoxy)cyclohexyl)-3-methylbut-2-enoate

A solution of ethyl 2-diethoxyphosphorylacetate (1.16 g, 5.17 mmol, 1.02 mL, 1.2 equiv.) in THE (15 mL) was degassed and purged with N₂ three times. The mixture was treated with LiHMDS (1 M, 6.46 mL, 1.5 equiv.) dropwise at −70° C. and stirred at −70° C. for 1 hour. Then, 1-((1r,4r)-4-(3-bromo-2-methylphenoxy)cyclohexyl)propan-2-one (1.4 g, 4.30 mmol, 1.0 equiv.) was added at −70° C. slowly. The mixture was warmed to 20° C. and stirred at 20° C. for 15 hours under N₂ atmosphere. The reaction mixture was added dropwise into saturated NH₄Cl solution (20 mL) at 0° C., and then extracted with EtOAc (30 mL×3). The combined organic layers were washed with brine (50 mL×1), dried over Na₂SO₄, filtered, and concentrated under reduced pressure to give a residue. The residue was purified by flash silica gel chromatography (0˜5% ethyl acetate/petroleum ether) to give ethyl (E)-4-((1r,4r)-4-(3-bromo-2-methylphenoxy)cyclohexyl)-3-methylbut-2-enoate (450 mg, 1.11 mmol, 25.86% yield) as a light yellow oil.

MS (ESI) m/z: 395.1 [M+H]⁺

¹H NMR (400 MHz, DMSO-d₆) δ=7.16-7.11 (m, 1H), 7.10-6.99 (m, 2H), 5.76-5.59 (m, 1H), 4.30-4.17 (m, 1H), 4.12-4.01 (m, 2H), 2.20 (s, 3H), 2.11-2.01 (m, 6H), 1.86 (s, 1H), 1.68 (d, J=12.8 Hz, 2H), 1.63-1.53 (m, 1H), 1.42-1.30 (m, 2H), 1.19 (t, J=7.2 Hz, 3H), 1.15-0.99 (m, 2H)

### Step H. Procedure for Preparation of ethyl 4-((1r,4r)-4-(3-bromo-2-methylphenoxy)cyclohexyl)-3-methylbutanoate

To a solution of ethyl (E)-4-((1r,4r)-4-(3-bromo-2-methylphenoxy)cyclohexyl)-3-methylbut-2-enoate (630 mg, 1.59 mmol, 1.0 equiv.) in EtOH (15 mL) was added PtO₂ (72.37 mg, 318.72 μmol, 0.2 equiv.) under N₂. The suspension was degassed under vacuum and purged with H₂ several times. The mixture was stirred under H₂ (15 psi) at 20° C. for 2 hours. The mixture was filtered to give a filtrate, which was concentrated under reduced pressure to give a residue. The residue was purified by reversed-phase HPLC (with 0.1% NH₃·H₂O) to give ethyl 4-((1r,4r)-4-(3-bromo-2-methylphenoxy)cyclohexyl)-3-methylbutanoate (440 mg, 1.01 mmol, 63.18% yield) as a yellow oil.

MS (ESI) m/z: 397.3 [M+H]⁺

¹H NMR (400 MHz, DMSO-d₆) δ=7.16-7.11 (m, 1H), 7.10-7.00 (m, 2H), 4.34 (t, J=4.8 Hz, 1H), 4.28-4.18 (m, 1H), 4.09-4.01 (m, 2H), 2.20 (s, 3H), 2.13-2.00 (m, 4H), 1.98-1.88 (m, 1H), 1.78-1.67 (m, 2H), 1.40-1.30 (m, 3H), 1.22-1.08 (m, 5H), 1.02-0.95 (m, 1H), 0.87 (d, J=6.8 Hz, 3H)

### Step I. Procedure for preparation of 4-((1r,4r)-4-(3-bromo-2-methylphenoxy)cyclohexyl)-3-methylbutan-1-ol

A solution of ethyl 4-((1r,4r)-4-(3-bromo-2-methylphenoxy)cyclohexyl)-3-methylbutanoate (440 mg, 1.11 mmol, 1.0 equiv.) in THE (5 mL) was degassed and purged with N₂ three times. LiAlH₄ (42.03 mg, 1.11 mmol, 1.0 equiv.) was then added. The mixture was stirred at 0° C. for 1 hour under N₂ atmosphere. To this mixture was added Na₂SO₄·10 H₂O (2 g) at 0° C. The resulting mixture was filtered to give a filtrate. The filtrate was concentrated under reduced pressure to give a residue. The residue was purified by prep-TLC (SiO₂, petroleum ether:ethyl acetate=8:1) to give 4-((1r,4r)-4-(3-bromo-2-methylphenoxy)cyclohexyl)-3-methylbutan-1-ol (300 mg, 806.85 mol, 72.86% yield) as a light yellow oil.

¹H NMR (400 MHz, DMSO-d₆) δ=7.16-7.11 (m, 1H), 7.10-7.03 (m, 1H), 7.03-6.99 (m, 1H), 4.32-4.17 (m, 2H), 3.48-3.34 (m, 2H), 2.21 (s, 3H), 2.04 (d, J=10.8 Hz, 2H), 1.81-1.67 (m, 2H), 1.66-1.55 (m, 1H), 1.48-1.30 (m, 4H), 1.24-1.10 (m, 2H), 1.07-0.92 (m, 3H), 0.83 (d, J=6.8 Hz, 3H)

### Step J. Procedure for Preparation of (S)-4-((1r,4s)-4-(3-bromo-2-methylphenoxy)cyclohexyl)-3-methylbutan-1-ol

Stereoisomers of 4-((1r,4r)-4-(3-bromo-2-methylphenoxy)cyclohexyl)-3-methylbutan-1-ol (300 mg, 844.34 μmol, 1 equiv.) were separated by prep-HPLC to give (S)-4-((1r,4s)-4-(3-bromo-2-methylphenoxy)cyclohexyl)-3-methylbutan-1-ol (150 mg, 408.03 μmol, 48.33% yield) as a light yellow oil.

### Step K. Procedure for Preparation of (S)-4-((1r,4s)-4-(3-bromo-2-methylphenoxy)cyclohexyl)-3-methylbutanal

A solution of (COCl)₂ (48.58 mg, 382.77 μmol, 33.51 μL, 2.0 equiv.) in DCM (2 mL) was degassed and purged with N₂ three times. The mixture was treated with DMSO (59.81 mg, 765.53 μmol, 59.81 μL, 4.0 equiv.) dropwise at −70° C., and then the mixture was stirred at −70° C. for 3 hours. (S)-4-((1r,4s)-4-(3-bromo-2-methylphenoxy)cyclohexyl)-3-methylbutan-1-ol (68 mg, 191.38 mol, 1.0 equiv.) in DCM (0.5 mL) was added dropwise at −70° C. The mixture was stirred at −70° C. for 1 hour, and then TEA (116.20 mg, 1.15 mmol, 159.83 μL, 6.0 equiv.) was added dropwise at −70° C. The resulting mixture was stirred at 20° C. for 0.5 hour. The reaction mixture was diluted with DCM (10 mL) and extracted with H₂O (10 mL×3). The combined organic layers were washed with saturated brine (20 mL×1), dried over Na₂SO₄, filtered, and concentrated under reduced pressure to give (S)-4-((1r,4s)-4-(3-bromo-2-methylphenoxy)cyclohexyl)-3-methylbutanal (55 mg, crude) as a yellow oil.

¹H NMR (400 MHz, DMSO-d₆) δ=9.66 (s, 1H), 7.16-7.11 (m, 1H), 7.11-6.99 (m, 2H), 4.29-4.17 (m, 1H), 2.42-2.36 (m, 1H), 2.21 (s, 4H), 2.12-2.00 (m, 3H), 1.82-1.69 (m, 2H), 1.42-1.29 (m, 3H), 1.15-1.08 (m, 2H), 1.05-0.94 (m, 2H), 0.88 (d, J=6.8 Hz, 3H)

### Step L. Procedure for Preparation of 3-(6-(4-((S)-4-((1r,4s)-4-(3-bromo-2-methylphenoxy)cyclohexyl)-3-methylbutyl)piperazin-1-yl)-1-methyl-1H-indazol-3-yl)piperidine-2,6-dione

A solution of (S)-4-((1r,4s)-4-(3-bromo-2-methylphenoxy)cyclohexyl)-3-methylbutanal (110 mg, 311.36 μmol, 1.0 equiv.) and 3-(1-methyl-6-piperazin-1-yl-indazol-3-yl)piperidine-2,6-dione (124.61 mg, 342.49 μmol, 1.1 equiv., HCl) in DCM (2 mL) was stirred at 20° C. for 16 hours. The mixture was then treated with NaBH(OAc)₃ (197.97 mg, 934.07 μmol, 3.0 equiv.). Afterwards, the mixture was stirred at 20° C. for 0.5 hour. The reaction mixture was diluted with H₂O (10 mL) and extracted with DCM (10 mL×3). The combined organic layers were washed with brine (20 mL×1), dried over Na₂SO₄, filtered, and concentrated under reduced pressure to give a residue. The residue was purified by flash silica gel chromatography (0˜4% methanol/dichloromethane) to give 3-(6-(4-((S)-4-((1r,4s)-4-(3-bromo-2-methylphenoxy)cyclohexyl)-3-methylbutyl)piperazin-1-yl)-1-methyl-1H-indazol-3-yl)piperidine-2,6-dione (128 mg, 180.06 mol, 57.83% yield) as a yellow oil.

MS (ESI) m/z: 666.1 [M+H]⁺.

¹H NMR (400 MHz, DMSO-d₆) δ=10.85 (s, 1H), 7.52 (d, J=8.0 Hz, 1H), 7.17-7.11 (m, 1H), 7.10-6.99 (m, 2H), 6.97-6.81 (m, 2H), 4.31-4.17 (m, 2H), 3.90 (s, 3H), 3.40-3.11 (m, 6H), 2.71-2.53 (m, 4H), 2.39-2.24 (m, 2H), 2.24-2.11 (m, 4H), 2.05 (d, J=10.0 Hz, 2H), 1.81-1.71 (m, 2H), 1.68-1.50 (m, 2H), 1.38-1.29 (m, 4H), 1.27-1.14 (m, 2H), 1.12-0.93 (m, 3H), 0.88 (d, J=5.6 Hz, 3H)

### Step M. Procedure for Preparation of tert-butyl 6-(8-(benzo[d]thiazol-2-ylcarbamoyl)-3,4-dihydroisoquinolin-2(1H)-yl)-3-(3-(((1s,4r)-4-((2S)-4-(4-(3-(2,6-dioxopiperidin-3-yl)-1-methyl-1H-indazol-6-yl)piperazin-1-yl)-2-methylbutyl)cyclohexyl)oxy)-2-methylphenyl)picolinate

A mixture of 3-(6-(4-((S)-4-((1r,4s)-4-(3-bromo-2-methylphenoxy)cyclohexyl)-3-methylbutyl)piperazin-1-yl)-1-methyl-1H-indazol-3-yl)piperidine-2,6-dione (128 mg, 192.58 mol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (129.76 mg, 211.83 mol, 1.1 equiv.), KF (33.56 mg, 577.73 μmol, 13.53 μL, 3.0 equiv.), and Ad₂nBuP Pd G₃ (14.02 mg, 19.26 μmol, 0.1 equiv.) in dioxane (3 mL) and H₂O (0.3 mL) was degassed and purged with N₂ three times. The mixture was stirred at 100° C. for 3 hours under N₂ atmosphere. The mixture was concentrated under reduced pressure to give a residue. The residue was purified by prep-TLC (SiO₂, petroleum ether:ethyl acetate=1:1) to give tert-butyl 6-(8-(benzo[d]thiazol-2-ylcarbamoyl)-3,4-dihydroisoquinolin-2(1H)-yl)-3-(3-(((1s,4r)-4-((2S)-4-(4-(3-(2,6-dioxopiperidin-3-yl)-1-methyl-1H-indazol-6-yl)piperazin-1-yl)-2-methylbutyl)cyclohexyl)oxy)-2-methylphenyl)picolinate (90 mg, crude) as a yellow oil.

MS (ESI) m/z: 1071.1 [M+H]⁺

### Step N. Procedure for Preparation of 6-(8-(benzo[d]thiazol-2-ylcarbamoyl)-3,4-dihydroisoquinolin-2(1H)-yl)-3-(3-(((1s,4r)-4-((2S)-4-(4-(3-(2,6-dioxopiperidin-3-yl)-1-methyl-1H-indazol-6-yl)piperazin-1-yl)-2-methylbutyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1S,4r)-4-((2S*)-4-(4-(3-(2,6-dioxopiperidin-3-yl)-1-methyl-1H-indazol-6-yl)piperazin-1-yl)-2-methylbutyl)cyclohexyl)oxy)-2-methylphenyl)picolinic acid]

To a solution of tert-butyl 6-(8-(benzo[d]thiazol-2-ylcarbamoyl)-3,4-dihydroisoquinolin-2(1H)-yl)-3-(3-(((1s,4r)-4-((2S)-4-(4-(3-(2,6-dioxopiperidin-3-yl)-1-methyl-1H-indazol-6-yl)piperazin-1-yl)-2-methylbutyl)cyclohexyl)oxy)-2-methylphenyl)picolinate (90 mg, 84.08 μmol, 1 equiv.) in DCM (2 mL) was added TFA (1 mL). The mixture was stirred at 40° C. for 10 min. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-((2S)-4-(4-(3-(2,6-dioxopiperidin-3-yl)-1-methyl-1H-indazol-6-yl)piperazin-1-yl)-2-methylbutyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1S,4r)-4-((2S*)-4-(4-(3-(2,6-dioxopiperidin-3-yl)-1-methyl-1H-indazol-6-yl)piperazin-1-yl)-2-methylbutyl)cyclohexyl)oxy)-2-methylphenyl)picolinic acid] (35.31 mg, 33.77 μmol, 40.16% yield) as a yellow solid.

MS (ESI) m/z: 1015.3 [M+H]⁺

¹H NMR (400 MHz, DMSO-d₆) δ=13.23-12.06 (m, 2H), 10.85 (s, 1H), 8.03 (d, J=7.6 Hz, 1H), 7.79 (d, J=8.0 Hz, 1H), 7.62 (d, J=7.6 Hz, 1H), 7.53-7.41 (m, 4H), 7.40-7.30 (m, 2H), 7.11-7.02 (m, 1H), 6.99-6.87 (m, 3H), 6.84 (s, 1H), 6.61 (d, J=7.6 Hz, 1H), 4.97 (s, 2H), 4.32-4.13 (m, 2H), 3.98-3.82 (m, 5H), 3.23 (s, 4H), 3.02 (s, 2H), 2.60 (s, 6H), 2.35-2.26 (m, 2H), 2.22-2.00 (m, 4H), 1.87 (s, 3H), 1.82-1.71 (m, 2H), 1.59-1.46 (m, 2H), 1.42-1.13 (m, 6H), 1.08-1.01 (m, 2H), 0.87 (d, J=5.6 Hz, 3H)

### Example 251. Preparation of Compound 307b

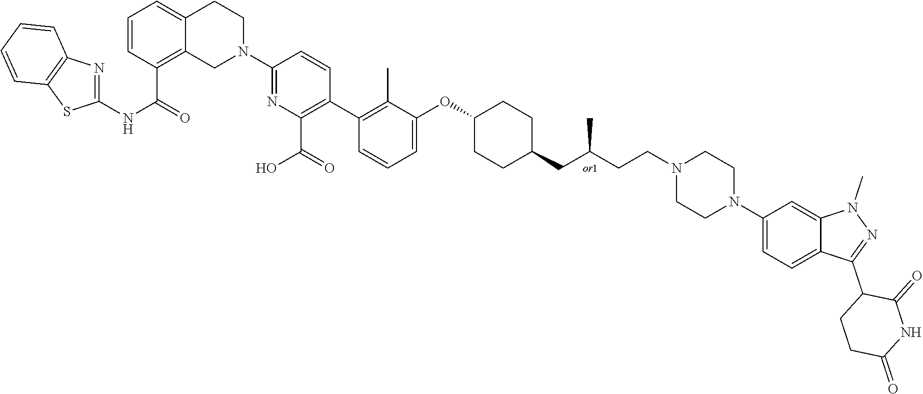 [SMILES: Cc1c(O[C@H]2CC[C@H](C[C@@H](C)CCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-((2R)-4-(4-(3-(2,6-dioxopiperidin-3-yl)-1-methyl-1H-indazol-6-yl)piperazin-1-yl)-2-methylbutyl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1R,4r)-4-((2R*)-4-(4-(3-(2,6-dioxopiperidin-3-yl)-1-methyl-1H-indazol-6-yl)piperazin-1-yl)-2-methylbutyl)cyclohexyl)oxy)-2-methylphenyl)picolinic acid]

### Step A. Procedure for Preparation of (R)-4-((1r,4r)-4-(3-bromo-2-methylphenoxy)cyclohexyl)-3-methylbutanal

A solution of (COCl)₂ (85.73 mg, 675.47 μmol, 59.13 μL, 2 equiv.) in DCM (2 mL) was cooled to −78° C., and DMSO (105.55 mg, 1.35 mmol, 105.55 μL, 4 equiv.) was added dropwise. The mixture was stirred at −78-25° C. for 2 hours under N₂ atmosphere. Then (R)-4-((1r,4R)-4-(3-bromo-2-methylphenoxy)cyclohexyl)-3-methylbutan-1-ol (120 mg, 337.73 μmol, 1 equiv.) was added dropwise, and the mixture was stirred at −78-25° C. for 2 hours under N₂ atmosphere. TEA (205.05 mg, 2.03 mmol, 282.05 μL, 6 equiv.) was added dropwise to the mixture at −78° C., and the mixture was warmed to 25° C. and stirred at 25° C. for 1 hour under N₂ atmosphere. The reaction mixture was diluted with H₂O (50 mL) and extracted with DCM (20 mL×3). The combined organic layers were washed with H₂O (10 mL×3), dried over Na₂SO₄, filtered, and concentrated under reduced pressure to give (R)-4-((1r,4r)-4-(3-bromo-2-methylphenoxy)cyclohexyl)-3-methylbutanal (110 mg, crude) was obtained as a yellow oil.

¹H NMR (400 MHz, DMSO-d₆) δ=9.66 (s, 1H), 7.17-6.78 (m, 3H), 4.29-4.12 (m, 1H), 2.43-2.34 (m, 1H), 2.21 (s, 3H), 2.12-1.99 (m, 3H), 1.82-1.67 (m, 2H), 1.36 (d, J=10.8 Hz, 2H), 1.24-0.97 (m, 6H), 0.88 (d, J=6.8 Hz, 3H).

### Step B. Procedure for Preparation of 3-(6-(4-((R)-4-((1r,4r)-4-(3-bromo-2-methylphenoxy)cyclohexyl)-3-methylbutyl)piperazin-1-yl)-1-methyl-1H-indazol-3-yl)piperidine-2,6-dione

A solution of (R)-4-((1r,4r)-4-(3-bromo-2-methylphenoxy)cyclohexyl)-3-methylbutanal (110 mg, 311.36 μmol, 1 equiv.) and 3-(1-methyl-6-(piperazin-1-yl)-1H-indazol-3-yl)piperidine-2,6-dione (122.32 mg, 373.63 μmol, 1.2 equiv.) in DCM (2 mL) was stirred at 0° C. for 2 hours. To this mixture was added NaBH(OAc)₃ (197.97 mg, 934.07 μmol, 3 equiv.). The resulting reaction mixture was stirred at 0° C. for 12 hours. The reaction mixture was quenched by addition of H₂O (5 mL) at 25° C., and extracted with DCM (5 mL×3). The combined organic layers were washed with brine (5 mL×3), dried over Na₂SO₄, filtered, and concentrated under reduced pressure to give 3-(6-(4-((R)-4-((1r,4r)-4-(3-bromo-2-methylphenoxy)cyclohexyl)-3-methylbutyl)piperazin-1-yl)-1-methyl-1H-indazol-3-yl)piperidine-2,6-dione (200 mg, crude) as a yellow oil.

MS (ESI) m/z: 664.2 [M+H]⁺.

### Step C. Procedure for Preparation of tert-butyl 6-(8-(benzo[d]thiazol-2-ylcarbamoyl)-3,4-dihydroisoquinolin-2(1H)-yl)-3-(3-(((1r,4r)-4-((2R)-4-(4-(3-(2,6-dioxopiperidin-3-yl)-1-methyl-1H-indazol-6-yl)piperazin-1-yl)-2-methylbutyl)cyclohexyl)oxy)-2-methylphenyl)picolinate

A mixture of 3-(6-(4-((r)-4-((1r,4R)-4-(3-bromo-2-methylphenoxy)cyclohexyl)-3-methylbutyl)piperazin-1-yl)-1-methyl-1H-indazol-3-yl)piperidine-2,6-dione (180 mg, 270.81 mol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (199.06 mg, 324.97 μmol, 1.2 equiv.), Ad₂nBup-Pd-G₃(cataCXium® A Pd G₃) (19.72 mg, 27.08 μmol, 0.1 equiv.), and KF (47.20 mg, 812.43 μmol, 60.18 μL, 3 equiv.) in dioxane (5 mL) and H₂O (0.5 mL) was degassed and purged with N₂ three times. The mixture was stirred at 100° C. for 2 hours under a N₂ atmosphere. The reaction mixture was filtered and concentrated under reduced pressure to give a residue. The residue was purified by prep-TLC (SiO₂, DCM:MeOH=20:1) to give tert-butyl 6-(8-(benzo[d]thiazol-2-ylcarbamoyl)-3,4-dihydroisoquinolin-2(1H)-yl)-3-(3-(((1r,4r)-4-((2R)-4-(4-(3-(2,6-dioxopiperidin-3-yl)-1-methyl-1H-indazol-6-yl)piperazin-1-yl)-2-methylbutyl)cyclohexyl)oxy)-2-methylphenyl)picolinate (170 mg, 150.89 μmol, 55.72% yield) as a yellow oil.

MS (ESI) m/z: 1070.6 [M+H]⁺.

### Step D. Procedure for Preparation of 6-(8-(benzo[d]thiazol-2-ylcarbamoyl)-3,4-dihydroisoquinolin-2(1H)-yl)-3-(3-(((1r,4r)-4-((2R)-4-(4-(3-(2,6-dioxopiperidin-3-yl)-1-methyl-1H-indazol-6-yl)piperazin-1-yl)-2-methylbutyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1R,4r)-4-((2R*)-4-(4-(3-(2,6-dioxopiperidin-3-yl)-1-methyl-1H-indazol-6-yl)piperazin-1-yl)-2-methylbutyl)cyclohexyl)oxy)-2-methylphenyl)picolinic acid]

A solution of tert-butyl 6-(8-(benzo[d]thiazol-2-ylcarbamoyl)-3,4-dihydroisoquinolin-2(1H)-yl)-3-(3-(((1r,4r)-4-((2R)-4-(4-(3-(2,6-dioxopiperidin-3-yl)-1-methyl-1H-indazol-6-yl)piperazin-1-yl)-2-methylbutyl)cyclohexyl)oxy)-2-methylphenyl)picolinate (170 mg, 158.83 μmol, 1 equiv.) in TFA (1 mL) and DCM (2 mL) was stirred at 25° C. for 12 hours. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1r,4r)-4-((2R)-4-(4-(3-(2,6-dioxopiperidin-3-yl)-1-methyl-1H-indazol-6-yl)piperazin-1-yl)-2-methylbutyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1R,4r)-4-((2R*)-4-(4-(3-(2,6-dioxopiperidin-3-yl)-1-methyl-1H-indazol-6-yl)piperazin-1-yl)-2-methylbutyl)cyclohexyl)oxy)-2-methylphenyl)picolinic acid] (48.43 mg, 47.06 μmol, 29.63% yield) as a white solid.

MS (ESI) m/z: 1014.5 [M+H]⁺.

¹H NMR (400 MHz, DMSO-d₆) δ=12.99-12.73 (m, 1H), 10.85 (s, 1H), 8.13 (s, 1H), 8.03 (d, J=8.0 Hz, 1H), 7.79 (d, J=8.0 Hz, 1H), 7.62 (d, J=6.8 Hz, 1H), 7.53-7.43 (m, 4H), 7.40-7.32 (m, 2H), 7.11-7.03 (m, 1H), 6.98-6.86 (m, 4H), 6.61 (d, J=7.6 Hz, 1H), 4.97 (s, 2H), 4.28-4.16 (m, 2H), 3.93-3.87 (m, 5H), 3.28-3.24 (m, 2H), 3.02 (s, 2H), 2.82-2.57 (m, 6H), 2.39-2.23 (m, 2H), 2.21-1.98 (m, 4H), 1.87 (s, 3H), 1.81-1.71 (m, 2H), 1.62-1.50 (m, 2H), 1.48-1.25 (m, 5H), 1.25-0.94 (m, 5H), 0.88 (d, J=5.6 Hz, 3H).

### Example 252. Preparation of Compound 308a

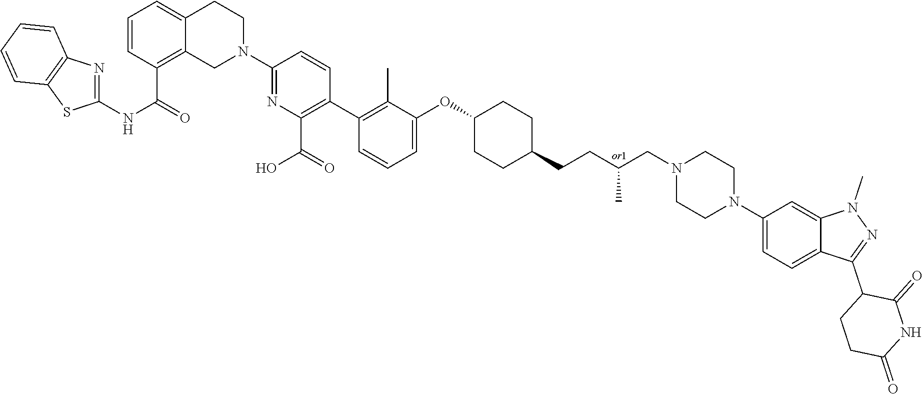 [SMILES: Cc1c(O[C@H]2CC[C@H](CC[C@@H](C)CN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-((3R)-4-(4-(3-(2,6-dioxopiperidin-3-yl)-1-methyl-1H-indazol-6-yl)piperazin-1-yl)-3-methylbutyl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1S,4r)-4-((3R*)-4-(4-(3-(2,6-dioxopiperidin-3-yl)-1-methyl-1H-indazol-6-yl)piperazin-1-yl)-3-methylbutyl)cyclohexyl)oxy)-2-methylphenyl)picolinic acid]

### Step A. Procedure for Preparation of 2-(4-(3-bromo-2-methylphenoxy)cyclohexyl)ethan-1-ol

To a solution of methyl 2-(4-(3-bromo-2-methylphenoxy)cyclohexyl)acetate (14.5 g, 42.49 mmol, 1 equiv.) in THE (150 mL) at 0° C. was added LiAlH₄ (1.94 g, 50.99 mmol, 1.2 equiv.). The mixture was stirred at 25° C. for 16 hours under a N₂ atmosphere. The reaction mixture was quenched by the addition of saturated sodium carbonate solution (6.0 mL) at 0° C., and then diluted with EtOAc (80 mL). The combined organic layers were filtered and concentrated under reduced pressure to give a residue. The residue was purified by flash silica gel chromatography (0-50% ethyl acetate/petroleum ether) and reverse-phase HPLC (with 0.1% formic acid) to give 2-(4-(3-bromo-2-methylphenoxy)cyclohexyl)ethan-1-ol (7.5 g, 23.58 mmol, 55.49% yield) as a purple oil.

MS (ESI) m/z: 314.2 [M+H]⁺

¹H NMR (400 MHz, DMSO-d₆) δ=7.15-7.10 (m, 1H), 7.06 (t, J=8.0 Hz, 1H), 7.00-6.96 (m, 1H), 4.63 (s, 1H), 4.33-4.29 (m, 1H), 3.46-3.42 (m, 2H), 2.28-2.27 (m, 1H), 2.26 (s, 3H), 2.20 (s, 1H), 1.91-1.83 (m, 2H), 1.53 (s, 1H), 1.49 (s, 2H), 1.37 (d, J=6.4 Hz, 2H), 1.22 (s, 2H)

### Step B. Procedure for Preparation of 2-((1r,4r)-4-(3-bromo-2-methylphenoxy)cyclohexyl)ethan-1-ol

The stereoisomers of 2-(4-(3-bromo-2-methylphenoxy)cyclohexyl)ethan-1-ol were separated by SFC to give 2-((1r,4r)-4-(3-bromo-2-methylphenoxy)cyclohexyl)ethan-1-ol (700 mg, 2.23 mmol, 23.33% yield) as a colorless oil.

¹H NMR (400 MHz, DMSO-d₆) δ=7.14-7.10 (m, 1H), 7.06 (t, J=8.0 Hz, 1H), 6.99-6.95 (m, 1H), 4.63 (s, 1H), 4.30 (t, J=5.2 Hz, 1H), 3.47-3.42 (m, 2H), 2.27-2.25 (m, 3H), 1.91-1.84 (m, 2H), 1.51 (dd, J=10.0, 6.8 Hz, 5H), 1.40-1.35 (m, 2H), 1.32-1.23 (m, 2H)

### Step C. Procedure for Preparation of 2-((1r,4r)-4-(3-bromo-2-methylphenoxy)cyclohexyl)acetaldehyde

To a solution of (COCl)₂ (405.22 mg, 3.19 mmol, 279.46 μL, 2 equiv.) in DCM (3 mL) was added DMSO (498.89 mg, 6.39 mmol, 498.89 μL, 4 equiv.) dropwise at −70° C. over 2 minutes. After addition, the mixture was stirred at this temperature for 30 minutes, and then 2-((1r,4r)-4-(3-bromo-2-methylphenoxy)cyclohexyl)ethan-1-ol (500.00 mg, 1.60 mmol, 1 equiv.) in DCM (3 mL) was added dropwise at −70° C. After addition, the mixture was stirred at this temperature for 30 minutes, and then TEA (969.15 mg, 9.58 mmol, 1.33 mL, 6 equiv.) was added into the mixture. The resulting mixture was stirred at 25° C. for 1 hour. The reaction mixture was diluted with water (20 mL) and extracted with DCM (25 mL×3). The combined organic layers were washed with brine (15 mL×3), dried over anhydrous sodium sulfate, filtered, and concentrated under reduced pressure to give a residue. The residue was purified by flash silica gel chromatography (0-7% ethyl acetate/petroleum ether) to give 2-((1r,4r)-4-(3-bromo-2-methylphenoxy)cyclohexyl)acetaldehyde (370 mg, 1.19 mmol, 74.48% yield) as a yellow oil.

¹H NMR (400 MHz, DMSO-d₆) δ=9.69-9.64 (m, 1H), 7.16-7.12 (m, 1H), 7.10-7.04 (m, 1H), 7.03-7.00 (m, 1H), 4.28-4.18 (m, 1H), 2.34 (dd, J=6.8, 2.0 Hz, 2H), 2.21 (s, 3H), 2.04 (d, J=9.6 Hz, 2H), 1.92-1.82 (m, 1H), 1.75 (d, J=12.0 Hz, 2H), 1.45-1.36 (m, 2H), 1.21-1.14 (m, 2H)

### Step D. Procedure for Preparation of ethyl (E)-4-((1r,4r)-4-(3-bromo-2-methylphenoxy)cyclohexyl)-2-methylbut-2-enoate

To a mixture of ethyl 2-(diethoxyphosphoryl)propanoate (424.82 mg, 1.78 mmol, 388.32 μL, 1.5 equiv.) was added NaH (104.62 mg, 2.62 mmol, 60% purity, 2.2 equiv.) in THE (5 mL) at −10° C. under N₂ atmosphere for 3 hours. Then 2-((1r,4r)-4-(3-bromo-2-methylphenoxy)cyclohexyl)acetaldehyde (370 mg, 1.19 mmol, 1 equiv.) was added at 0° C. under N₂ atmosphere. The mixture was stirred at 25° C. for 16 hours under N₂ atmosphere. The reaction mixture was quenched by addition water (30 mL) at 0° C. and extracted with EtOAc (20 mL×3). The combined organic layers were washed with brine (20 mL×3), dried over anhydrous sodium sulfate filtered, and concentrated under reduced pressure to give a residue. The residue was purified by flash silica gel chromatography (0-7% ethyl acetate/petroleum ether) to give ethyl (E)-4-((1r,4r)-4-(3-bromo-2-methylphenoxy)cyclohexyl)-2-methylbut-2-enoate (200 mg, 505.91 μmol, 42.55% yield) as a colorless oil.

MS (ESI) m/z: 396.9 [M+H]⁺

¹H NMR (400 MHz, DMSO-d₆) δ=7.16-7.11 (m, 1H), 7.09-7.04 (m, 1H), 7.03-7.00 (m, 1H), 6.71 (t, J=7.6 Hz, 1H), 4.28-4.19 (m, 1H), 4.12 (q, J=7.2 Hz, 2H), 2.20 (s, 3H), 2.11 (t, J=7.2 Hz, 2H), 2.04 (d, J=10.0 Hz, 2H), 1.78-1.76 (m, 3H), 1.72 (s, 2H), 1.51-1.44 (m, 1H), 1.42-1.32 (m, 2H), 1.24-1.19 (m, 3H), 1.18-1.07 (m, 2H)

### Step E. Procedure for Preparation of ethyl 4-((1r,4s)-4-(3-bromo-2-methylphenoxy)cyclohexyl)-2-methylbutanoate

A mixture of ethyl (E)-4-((1r,4r)-4-(3-bromo-2-methylphenoxy)cyclohexyl)-2-methylbut-2-enoate (200 mg, 505.91 μmol, 1 equiv.) and PtO₂ (57.44 mg, 50.59 μmol, 20% purity, 0.1 equiv.) in EtOH (10 mL) was degassed and purged with N₂ three times. The mixture was stirred at 25° C. for 16 hours under H₂ atmosphere. The reaction mixture was filtered and concentrated under reduced pressure to give ethyl 4-((1r,4s)-4-(3-bromo-2-methylphenoxy)cyclohexyl)-2-methylbutanoate (170 mg, 408.72 μmol, 80.79% yield) as a colorless oil.

MS (ESI) m/z: 397.2 [M+H]⁺

¹H NMR (400 MHz, DMSO-d₆) δ=7.15-7.10 (m, 1H), 7.06 (t, J=8.0 Hz, 1H), 7.03-6.98 (m, 1H), 4.38 (s, 1H), 4.26-4.17 (m, 1H), 4.09-4.02 (m, 2H), 3.44 (q, J=7.2 Hz, 2H), 2.41-2.31 (m, 1H), 2.22-2.17 (m, 3H), 2.03 (d, J=9.6 Hz, 2H), 1.78-1.70 (m, 2H), 1.59-1.50 (m, 1H), 1.44-1.37 (m, 1H), 1.36-1.28 (m, 2H), 1.16 (s, 4H), 1.05 (d, J=2.0 Hz, 4H)

### Step F. Procedure for Preparation of 4-((1r,4s)-4-(3-bromo-2-methylphenoxy)cyclohexyl)-2-methylbutan-1-ol

A solution of ethyl 4-((1r,4s)-4-(3-bromo-2-methylphenoxy)cyclohexyl)-2-methylbutanoate (170 mg, 427.84 μmol, 1 equiv.) in THF (5 mL) at 0° C. was stirred at 0° C. for 15 minutes. After 15 minutes, LiAlH₄ (32.47 mg, 855.68 μmol, 2 equiv.) was added, and the mixture was stirred at 25° C. for 16 hours under N₂ atmosphere. The reaction mixture was quenched by the addition of saturated sodium carbonate solution (0.15 mL) at 0° C. and then diluted with EtOAc 20 mL. The combined organic layers were filtered and concentrated under reduced pressure to give a residue. The residue was purified by prep-TLC (SiO₂, petroleum ether: ethyl acetate=3:1) and prep-HPLC to give 4-((1r,4s)-4-(3-bromo-2-methylphenoxy)cyclohexyl)-2-methylbutan-1-ol (100 mg, 280.58 mol, 65.58% yield, 99.693% purity) as a yellow oil.

MS (ESI) m/z: 356.4 [M+H]⁺

¹H NMR (400 MHz, CDCl₃) δ=7.14 (d, J=8.0 Hz, 1H), 6.97 (t, J=8.0 Hz, 1H), 6.81 (d, J=8.0 Hz, 1H), 4.14-4.04 (m, 1H), 3.55-3.49 (m, 1H), 3.48-3.42 (m, 1H), 2.32-2.29 (m, 3H), 2.17-2.10 (m, 2H), 1.86 (td, J=10.0, 2.8 Hz, 2H), 1.65-1.55 (m, 1H), 1.49-1.43 (m, 4H), 1.31-1.21 (m, 3H), 1.18-1.11 (m, 1H), 1.07-0.98 (m, 2H), 0.93 (d, J=6.4 Hz, 3H)

### Step G. Procedure for Preparation of (R)-4-((1r,4s)-4-(3-bromo-2-methylphenoxy)cyclohexyl)-2-methylbutan-1-ol

The stereoisomers of 4-((1r,4s)-4-(3-bromo-2-methylphenoxy)cyclohexyl)-2-methylbutan-1-ol were separated by SFC to give (R)-4-((1r,4s)-4-(3-bromo-2-methylphenoxy)cyclohexyl)-2-methylbutan-1-ol (40 mg, 112.58 μmol, 40.00% yield) was obtained as a colorless oil.

### Step H. Procedure for Preparation of (R)-4-((1r,4s)-4-(3-bromo-2-methylphenoxy)cyclohexyl)-2-methylbutanal

To a solution of (R)-4-((1r,4s)-4-(3-bromo-2-methylphenoxy)cyclohexyl)-2-methylbutan-1-ol (40 mg, 112.58 μmol, 1 equiv.) in DCM (1 mL) was added Dess-Martin periodinane (95.50 mg, 225.16 mol, 69.76 μL, 2 equiv.). The mixture was stirred at 25° C. for 1 hour. The reaction mixture was diluted with water (10 mL) and extracted with DCM (10 mL×3). The combined organic layers were dried over sodium sulfate, filtered, and concentrated under reduced pressure to give (R)-4-((1r,4s)-4-(3-bromo-2-methylphenoxy)cyclohexyl)-2-methylbutanal (45 mg, crude) as a yellow oil.

### Step I. Procedure for preparation of 3-(6-(4-((R)-4-((1r,4s)-4-(3-bromo-2-methylphenoxy)cyclohexyl)-2-methylbutyl)piperazin-1-yl)-1-methyl-1H-indazol-3-yl)piperidine-2,6-dione

To a solution of (R)-4-((1r,4s)-4-(3-bromo-2-methylphenoxy)cyclohexyl)-2-methylbutanal (45 mg, 127.37 μmol, 1 equiv.) in DCM (2 mL) was added 3-(1-methyl-6-piperazin-1-yl-indazol-3-yl)piperidine-2,6-dione (41.70 mg, 127.37 μmol, 1 equiv.) at 25° C. The reaction mixture was stirred for 15 hours. To this mixture, NaBH(OAc)₃ (80.99 mg, 382.12 μmol, 3 equiv.) was added at 0° C. The resulting mixture was stirred at 25° C. for 1 hour. The residue was concentrated under reduced pressure to give a residue. The residue was purified by prep-TLC (SiO₂, DCM:MeOH=10:1) to give 3-(6-(4-((R)-4-((1r,4s)-4-(3-bromo-2-methylphenoxy)cyclohexyl)-2-methylbutyl)piperazin-1-yl)-1-methyl-1H-indazol-3-yl)piperidine-2,6-dione (60 mg, crude) as a white solid.

MS (ESI) m/z: 664.5 [M+H]⁺.

### Step J. Procedure for Preparation of tert-butyl 6-(8-(benzo[d]thiazol-2-ylcarbamoyl)-3,4-dihydroisoquinolin-2(1H)-yl)-3-(3-(((1s,4r)-4-((3R)-4-(4-(3-(2,6-dioxopiperidin-3-yl)-1-methyl-1H-indazol-6-yl)piperazin-1-yl)-3-methylbutyl)cyclohexyl)oxy)-2-methylphenyl)picolinate

A mixture of 3-(6-(4-((R)-4-((1r,4s)-4-(3-bromo-2-methylphenoxy)cyclohexyl)-2-methylbutyl)piperazin-1-yl)-1-methyl-1H-indazol-3-yl)piperidine-2,6-dione (41 mg, 61.68 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (37.78 mg, 61.68 μmol, 1 equiv.), KF (10.75 mg, 185.05 μmol, 4.34 μL, 3 equiv.), and [2-(2-aminophenyl)phenyl]palladium(1⁺); bis(1-adamantyl)-butyl-phosphane; methanesulfonate (4.49 mg, 6.17 μmol, 0.1 equiv.) in dioxane (5 mL) and H₂O (0.5 mL) was degassed and purged with N₂ three times. The mixture was stirred at 100° C. for 2 hours under N₂ atmosphere. The reaction mixture was diluted with water (10 mL) and extracted with ethyl acetate (10 mL×3). The combined organic layers were dried over Na₂SO₄, filtered, and concentrated under reduced pressure to give a residue. The residue was purified by prep-TLC (SiO₂, DCM:MeOH=10:1) to give tert-butyl 6-(8-(benzo[d]thiazol-2-ylcarbamoyl)-3,4-dihydroisoquinolin-2(1H)-yl)-3-(3-(((1s,4r)-4-((3R)-4-(4-(3-(2,6-dioxopiperidin-3-yl)-1-methyl-1H-indazol-6-yl)piperazin-1-yl)-3-methylbutyl)cyclohexyl)oxy)-2-methylphenyl)picolinate (60 mg, 56.06 μmol, 90.88% yield) as a white solid.

MS (ESI) m/z: 536.3 [M+H]⁺.

¹H NMR (400 MHz, DMSO-d₆) δ=12.85 (d, J=2.8 Hz, 1H), 10.83 (s, 1H), 8.01 (d, J=7.6 Hz, 1H), 7.77 (d, J=7.2 Hz, 1H), 7.60 (d, J=6.4 Hz, 1H), 7.52-7.41 (m, 4H), 7.40-7.30 (m, 2H), 7.11-7.03 (m, 1H), 6.98-6.88 (m, 3H), 6.83 (s, 1H), 6.55 (d, J=6.8 Hz, 1H), 4.97 (s, 2H), 4.29-4.16 (m, 2H), 3.88 (s, 5H), 3.21 (s, 4H), 3.06-3.00 (m, 2H), 2.69-2.58 (m, 4H), 2.33 (s, 3H), 2.22-2.14 (m, 3H), 2.09-2.01 (m, 3H), 1.84 (s, 3H), 1.81-1.72 (m, 2H), 1.70-1.59 (m, 2H), 1.27-1.17 (m, 4H), 1.07 (s, 3H), 0.99 (s, 9H), 0.88 (d, J=6.0 Hz, 3H)

### Step K. Procedure for Preparation of 6-(8-(benzo[d]thiazol-2-ylcarbamoyl)-3,4-dihydroisoquinolin-2(1H)-yl)-3-(3-(((1s,4r)-4-((3R)-4-(4-(3-(2,6-dioxopiperidin-3-yl)-1-methyl-1H-indazol-6-yl)piperazin-1-yl)-3-methylbutyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1S,4r)-4-((3R*)-4-(4-(3-(2,6-dioxopiperidin-3-yl)-1-methyl-1H-indazol-6-yl)piperazin-1-yl)-3-methylbutyl)cyclohexyl)oxy)-2-methylphenyl)picolinic acid]

A solution of tert-butyl 6-(8-(benzo[d]thiazol-2-ylcarbamoyl)-3,4-dihydroisoquinolin-2(1H)-yl)-3-(3-(((1s,4r)-4-((3R)-4-(4-(3-(2,6-dioxopiperidin-3-yl)-1-methyl-1H-indazol-6-yl)piperazin-1-yl)-3-methylbutyl)cyclohexyl)oxy)-2-methylphenyl)picolinate (55 mg, 51.39 μmol, 1 equiv.) in DCM (3 mL) and TFA (1 mL) was stirred at 25° C. for 12 hours. The residue was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-((3R)-4-(4-(3-(2,6-dioxopiperidin-3-yl)-1-methyl-1H-indazol-6-yl)piperazin-1-yl)-3-methylbutyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1S,4r)-4-((3R*)-4-(4-(3-(2,6-dioxopiperidin-3-yl)-1-methyl-1H-indazol-6-yl)piperazin-1-yl)-3-methylbutyl)cyclohexyl)oxy)-2-methylphenyl)picolinic acid] (20.39 mg, 19.74 μmol, 80.10% yield) as a yellow solid.

MS (ESI) m/z: 1014.6 [M+H]⁺.

¹H NMR (400 MHz, DMSO-d₆) δ=10.85 (s, 1H), 8.17 (s, 1H), 8.03 (d, J=7.2 Hz, 1H), 7.81-7.75 (m, 1H), 7.62 (d, J=6.4 Hz, 1H), 7.51-7.41 (m, 4H), 7.40-7.32 (m, 2H), 7.10-7.02 (m, 1H), 6.96-6.87 (m, 3H), 6.84 (s, 1H), 6.61 (d, J=6.8 Hz, 1H), 5.02-4.92 (m, 2H), 4.28-4.16 (m, 2H), 3.93-3.87 (m, 5H), 3.21 (s, 4H), 3.02 (s, 2H), 2.63-2.58 (m, 3H), 2.22-2.01 (m, 7H), 1.87 (s, 3H), 1.79 (d, J=2.8 Hz, 2H), 1.66 (dt, J=2.4, 4.4 Hz, 1H), 1.50-1.30 (m, 4H), 1.27-1.17 (m, 3H), 1.14-0.95 (m, 4H), 0.88 (d, J=6.0 Hz, 3H).

### Example 253. Preparation of Compound 309

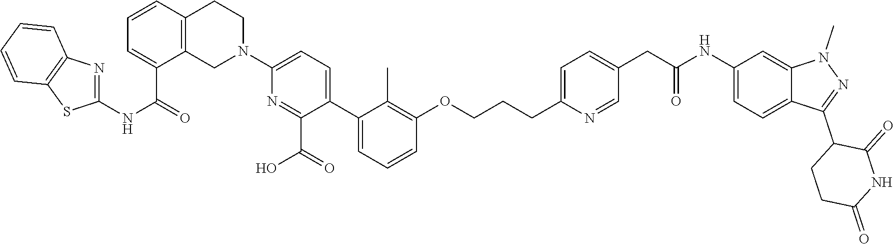 [SMILES: Cc1c(OCCCc2ccc(CC(=O)Nc3ccc4c(C5CCC(=O)NC5=O)nn(C)c4c3)cn2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[5-[2-[[3-(2,6-dioxo-3-piperidyl)-1-methyl-indazol-6-yl]amino]-2-oxo-ethyl]-2-pyridyl]propoxy]-2-methyl-phenyl]pyridine-2-carboxylic acid

### Step A. Procedure for Preparation of 1-bromo-2-methyl-3-prop-2-ynoxy-benzene

A mixture of 3-bromo-2-methyl-phenol (4 g, 21.39 mmol, 1 equiv.), 3-bromoprop-1-yne (3.82 g, 25.66 mmol, 2.77 mL, 1.2 equiv.), K₂CO₃ (8.87 g, 64.16 mmol, 3 equiv.), and KI (355.02 mg, 2.14 mmol, 0.1 equiv.) in THE (30 mL) was degassed and purged with N₂ three times. The mixture was stirred at 60° C. for 16 hours under N₂ atmosphere. The reaction mixture was concentrated under reduced pressure to give a residue. The residue was purified by flash silica gel chromatography (0-3% ethyl acetate/petroleum ether) to give 1-bromo-2-methyl-3-prop-2-ynoxy-benzene (4.12 g, 18.30 mmol, 85.59% yield) as a colorless oil.

¹H NMR (400 MHz, CDCl₃) δ=7.24-7.19 (m, 1H), 7.03 (t, J=8.0 Hz, 1H), 6.92 (d, J=8.4 Hz, 1H), 4.72 (d, J=2.4 Hz, 2H), 2.53 (s, 1H), 2.35 (s, 3H)

### Step B. Procedure for Preparation of methyl 2-[6-[3-(3-bromo-2-methyl-phenoxy)prop-1-ynyl]-3-pyridyl]acetate

A mixture of 1-bromo-2-methyl-3-prop-2-ynoxy-benzene (1.82 g, 8.08 mmol, 1.5 equiv.), methyl 2-(6-chloro-3-pyridyl)acetate (1 g, 5.39 mmol, 1 equiv.), K₂CO₃ (2.23 g, 16.16 mmol, 3 equiv.), acetonitrile; dichloropalladium (27.95 mg, 107.75 μmol, 0.02 equiv.), and XPhos (154.11 mg, 323.26 μmol, 0.06 equiv.) in MeCN (10 mL) was degassed and purged with N₂ three times. The mixture was stirred at 60° C. for 1 hour under N₂ atmosphere. The reaction mixture was diluted with water (20 mL) and extracted with EtOAc (25 mL×3). The combined organic layers were washed with brine (15 mL×3), dried over anhydrous sodium sulfate, filtered, and concentrated under reduced pressure to give a residue. The residue was purified by flash silica gel chromatography (0-25% ethyl acetate/petroleum ether) to give methyl 2-[6-[3-(3-bromo-2-methyl-phenoxy)prop-1-ynyl]-3-pyridyl]acetate (180 mg, 480.99 μmol, 8.93% yield) as a brown oil.

MS (ESI) m/z: 375.7 [M+H]⁺

¹H NMR (400 MHz, CDCl₃) δ=7.66-7.58 (m, 1H), 7.44-7.36 (m, 1H), 7.23-7.17 (m, 1H), 7.07-6.98 (m, 3H), 4.96-4.91 (m, 2H), 3.72 (s, 3H), 3.65 (s, 2H), 2.36 (s, 3H)

### Step C. Procedure for Preparation of methyl 2-[6-[3-(3-bromo-2-methyl-phenoxy)propyl]-3-pyridyl]acetate

A mixture of methyl 2-[6-[3-(3-bromo-2-methyl-phenoxy)prop-1-ynyl]-3-pyridyl]acetate (160 mg, 427.55 μmol, 1 equiv.) and Rh/Al₂O₃ (88.00 mg, 42.75 μmol, 5% purity, 0.1 equiv.) in EtOAc (10 mL) was degassed and purged with H₂ three times. The reaction mixture was stirred at 25° C. for 3 hours under H₂ atmosphere. The reaction mixture was filtered and concentrated under reduced pressure to give a residue. The residue was purified by flash silica gel chromatography (0-5% DCM/MeOH) to give methyl 2-[6-[3-(3-bromo-2-methyl-phenoxy)propyl]-3-pyridyl]acetate (65 mg, 171.84 μmol, 40.19% yield) as a brown oil.

MS (ESI) m/z: 379.9 [M+H]⁺

¹H NMR (400 MHz, CDCl₃) δ=8.46 (s, 1H), 7.65-7.58 (m, 1H), 7.19 (d, J=7.2 Hz, 1H), 7.15 (d, J=8.0 Hz, 1H), 7.02-6.96 (m, 1H), 6.75 (d, J=8.4 Hz, 1H), 4.01 (t, J=6.0 Hz, 2H), 3.63 (s, 3H), 3.03 (t, J=7.2 Hz, 2H), 2.33-2.30 (m, 3H), 2.28-2.21 (m, 2H), 1.31-1.25 (m, 2H)

### Step D. Procedure for Preparation of tert-butyl 6-[8-(1,3-benzothiazol-2-ylcarbamoyl)-3,4-dihydro-1H-isoquinolin-2-yl]-3-[3-[3-[5-(2-methoxy-2-oxo-ethyl)-2-pyridyl]propoxy]-2-methyl-phenyl]pyridine-2-carboxylate

tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (116.60 mg, 190.35 μmol, 1.2 equiv.), methyl 2-[6-[3-(3-bromo-2-methyl-phenoxy)propyl]-3-pyridyl]acetate (60 mg, 158.62 mol, 1 equiv.), Ad₂nBuP Pd G₃ (11.55 mg, 15.86 μmol, 0.1 equiv.), and KF (1.5 M, 317.24 μL, 3 equiv.) were taken up into a microwave tube in dioxane (1 mL), and the tube was sealed. The sealed tube was heated at 100° C. for 1 hour under microwave. The reaction mixture was diluted with water (20 mL) and extracted with EtOAc (25 mL×3). The combined organic layers were washed with brine (15 mL×3), dried over anhydrous sodium sulfate, filtered, and concentrated under reduced pressure to give a residue. The residue was purified by prep-TLC (SiO₂, DCM:MeOH=15:1) to give tert-butyl 6-[8-(1,3-benzothiazol-2-ylcarbamoyl)-3,4-dihydro-1H-isoquinolin-2-yl]-3-[3-[3-[5-(2-methoxy-2-oxo-ethyl)-2-pyridyl]propoxy]-2-methyl-phenyl]pyridine-2-carboxylate (50 mg, 63.78 μmol, 40.21% yield) as a brown oil.

MS (ESI) m/z: 784.7 [M+H]⁺

### Step E. Procedure for Preparation of 2-[6-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-3-pyridyl]acetic acid

A mixture of tert-butyl 6-[8-(1,3-benzothiazol-2-ylcarbamoyl)-3,4-dihydro-1H-isoquinolin-2-yl]-3-[3-[3-[5-(2-methoxy-2-oxo-ethyl)-2-pyridyl]propoxy]-2-methyl-phenyl]pyridine-2-carboxylate (50 mg, 63.78 μmol, 1 equiv.) and LiOH H₂O (8.03 mg, 191.34 μmol, 3 equiv.) in THE (2 mL) and H₂O (0.5 mL) was degassed and purged with N₂ three times. The mixture was stirred at 25° C. for 2 hours under N₂ atmosphere. The reaction mixture was diluted with water (20 mL) and extracted with DCM (25 mL×3). The combined organic layers were washed with brine (15 mL×3), dried over anhydrous sodium sulfate, filtered, and concentrated under reduced pressure to give 2-[6-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-3-pyridyl]acetic acid (50 mg, crude) as a brown solid.

MS (ESI) m/z: 770.7 [M+H]⁺

### Step F. Procedure for Preparation of tert-butyl 6-[8-(1,3-benzothiazol-2-ylcarbamoyl)-3,4-dihydro-1H-isoquinolin-2-yl]-3-[3-[3-[5-[2-[[3-(2,6-dioxo-3-piperidyl)-1-methyl-indazol-6-yl]amino]-2-oxo-ethyl]-2-pyridyl]propoxy]-2-methyl-phenyl]pyridine-2-carboxylate

A mixture of 3-(6-amino-1-methyl-indazol-3-yl)piperidine-2,6-dione (25.16 mg, 97.41 μmol, 1.5 equiv.), 2-[6-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-3-pyridyl]acetic acid (50 mg, 64.94 mol, 1 equiv.), and EDCI (18.67 mg, 97.41 μmol, 1.5 equiv.) in pyridine (2 mL) was degassed and purged with N₂ three times. The mixture was stirred at 25° C. for 3 hours under N₂ atmosphere. The reaction mixture was diluted with water (20 mL) and extracted with EtOAc (25 mL×3). The combined organic layers were washed with brine (15 mL×3), dried over anhydrous sodium sulfate, filtered, and concentrated under reduced pressure to give tert-butyl 6-[8-(1,3-benzothiazol-2-ylcarbamoyl)-3,4-dihydro-1H-isoquinolin-2-yl]-3-[3-[3-[5-[2-[[3-(2,6-dioxo-3-piperidyl)-1-methyl-indazol-6-yl]amino]-2-oxo-ethyl]-2-pyridyl]propoxy]-2-methyl-phenyl]pyridine-2-carboxylate (70 mg, crude) as a green-black oil.

MS (ESI) m/z: 1010.4 [M+H]⁺

### Step G. Procedure for Preparation of 6-[8-(1,3-benzothiazol-2-ylcarbamoyl)-3,4-dihydro-1H-isoquinolin-2-yl]-3-[3-[3-[5-[2-[[3-(2,6-dioxo-3-piperidyl)-1-methyl-indazol-6-yl]amino]-2-oxo-ethyl]-2-pyridyl]propoxy]-2-methyl-phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[3-[3-[5-[2-[[3-(2,6-dioxo-3-piperidyl)-1-methyl-indazol-6-yl]amino]-2-oxo-ethyl]-2-pyridyl]propoxy]-2-methyl-phenyl]pyridine-2-carboxylate (70 mg, 69.30 μmol, 1 equiv.) in DCM (2 mL) and TFA (2 mL) was degassed and purged with N₂ three times. The mixture was stirred at 40° C. for 24 hours under N₂ atmosphere. The reaction mixture was filtered and concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[3-[5-[2-[[3-(2,6-dioxo-3-piperidyl)-1-methyl-indazol-6-yl]amino]-2-oxo-ethyl]-2-pyridyl]propoxy]-2-methyl-phenyl]pyridine-2-carboxylic acid (16.1 mg, 15.81 μmol, 22.82% yield) as a white solid.

MS (ESI) m/z: 954.6 [M+H]⁺

¹H NMR (400 MHz, DMSO-d₆) δ=12.90-12.40 (m, 1H), 10.91-10.82 (m, 1H), 10.44-10.38 (m, 1H), 8.47-8.42 (m, 1H), 8.18-8.15 (m, 1H), 8.07-7.98 (m, 2H), 7.79 (d, J=7.6 Hz, 1H), 7.69-7.58 (m, 3H), 7.50-7.41 (m, 3H), 7.40-7.32 (m, 2H), 7.26 (d, J=7.6 Hz, 1H), 7.13-7.04 (m, 2H), 6.98-6.92 (m, 1H), 6.90-6.82 (m, 1H), 6.67-6.61 (m, 1H), 4.98 (s, 2H), 4.34-4.27 (m, 1H), 4.05-3.98 (m, 2H), 3.95-3.86 (m, 5H), 3.70 (s, 2H), 3.05-2.99 (m, 2H), 2.91 (t, J=7.2 Hz, 2H), 2.71-2.61 (m, 2H), 2.16 (d, J=8.0 Hz, 2H), 1.91 (s, 3H), 1.24 (s, 2H)

### Example 254. Preparation of Compound 311a

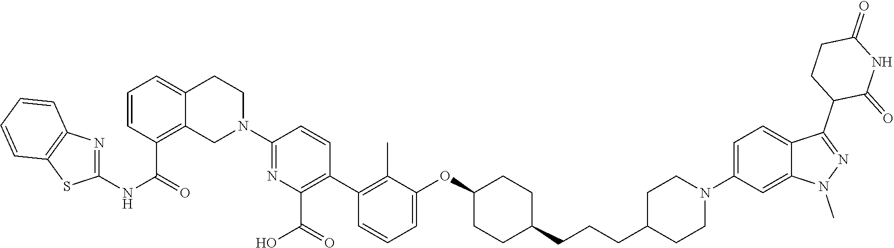 [SMILES: Cc1c(O[C@H]2CC[C@@H](CCCC3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4s)-4-(3-(1-(3-(2,6-dioxopiperidin-3-yl)-1-methyl-1H-indazol-6-yl)piperidin-4-yl)propyl)cyclohexyl)oxy)-2-methylphenyl) picolinic acid

### Step A. Procedure for Preparation of methyl 2-(4-hydroxycyclohexyl)acetate

To a solution of 2-(4-hydroxycyclohexyl)acetic acid (10 g, 63.21 mmol, 1 equiv.) in MeOH (100 mL) was added H₂SO₄ (12.40 g, 126.43 mmol, 6.74 mL, 2 equiv.) at 0° C. The mixture was stirred at 60° C. for 3 hours. The reaction mixture was quenched with NaHCO₃ (300 mL) and extracted with EtOAc (50 mL×6). The combined organic layers were washed with saturated salt solution (100 mL×1), dried over Na₂SO₄, filtered, and concentrated under reduced pressure to give methyl 2-(4-hydroxycyclohexyl)acetate (13.5 g, crude) as a light yellow oil.

### Step B. Procedure for Preparation of methyl 2-[4-(3-bromo-2-methyl-phenoxy)cyclohexyl]acetate

A mixture of methyl 2-(4-hydroxycyclohexyl)acetate (12 g, 69.68 mmol, 1 equiv.), 3-bromo-2-methyl-phenol (15.64 g, 83.61 mmol, 1.2 equiv.), and 2-(tributyl-λ5-phosphanylidene)acetonitrile (25.23 g, 104.52 mmol, 1.5 equiv.) in toluene (120 mL) was degassed and purged with N₂ three times. The mixture was stirred at 120° C. for 12 hours under N₂ atmosphere. The reaction mixture was concentrated under reduced pressure to give a residue. The residue was purified by flash silica gel chromatography (0-5% ethyl acetate/petroleum ether) to give methyl 2-[4-(3-bromo-2-methyl-phenoxy)cyclohexyl]acetate (20 g, crude) as a yellow oil.

### Step C. Procedure for Preparation of 2-[4-(3-bromo-2-methyl-phenoxy)cyclohexyl]ethanol

To a solution of methyl 2-[4-(3-bromo-2-methyl-phenoxy)cyclohexyl]acetate (20 g, 58.61 mmol, 1 equiv.) in THE (10 mL) was added LiAlH₄ (2.67 g, 70.33 mmol, 1.2 equiv.) at 0° C. The mixture was stirred at 0° C. for 1 hour. The reaction mixture was quenched by sodium sulfate decahydrate (10 g) at 0° C., filtered, and concentrated under reduced pressure to give a residue. The residue was purified by flash silica gel chromatography (0˜20% ethyl acetate/petroleum ether) to give 2-[4-(3-bromo-2-methyl-phenoxy)cyclohexyl]ethanol (7 g, crude) as a yellow oil.

¹H NMR (400 MHz, CDCl₃) δ=7.16-7.10 (m, 1H), 6.97 (t, J=8.0 Hz, 1H), 6.83-6.75 (m, 1H), 4.55-4.04 (m, 1H), 3.75-3.71 (m, 2H), 2.35-2.30 (m, 3H), 2.18-2.10 (m, 1H), 2.05-1.98 (m, 2H), 1.92-1.84 (m, 1H), 1.60 (d, J=2.0 Hz, 1H), 1.57-1.53 (m, 3H), 1.47-1.34 (m, 3H), 1.14-1.01 (m, 1H).

### Step D. Procedure for Preparation of 2-((1s,4s)-4-(3-bromo-2-methylphenoxy)cyclohexyl)acetaldehyde

To a solution of DMSO (6.98 g, 89.39 mmol, 6.98 mL, 4 equiv.) in DCM (20 mL) was added oxalyl dichloride (5.67 g, 44.70 mmol, 3.91 mL, 2 equiv.) in DCM (5 mL). The mixture was stirred at −70° C. for 1 hour. 2-[4-(3-bromo-2-methyl-phenoxy)cyclohexyl]ethanol (7 g, 22.35 mmol, 1 equiv.) in DCM (5 mL) was added to the mixture at −70° C. and stirred for 1 hour. TEA (13.57 g, 134.09 mmol, 18.66 mL, 6 equiv.) was added into the mixture at −70° C. The mixture was stirred at −70° C. for 1 hour. The reaction mixture was quenched by 50 mL H₂O and extracted with DCM 150 mL (50 mL×3). The combined organic layers were washed with saturated salt solution (20 mL×1), dried over anhydrous Na₂SO₄, filtered, and concentrated under reduced pressure to give a residue. The residue was purified by flash silica gel chromatography (0˜5% ethyl acetate/petroleum ether) to give 2-((1s,4s)-4-(3-bromo-2-methylphenoxy)cyclohexyl)acetaldehyde (4 g, crude) as a light yellow oil.

¹H NMR (400 MHz, CDCl₃) δ=9.79 (t, J=2.0 Hz, 1H), 7.15 (d, J=8.0 Hz, 1H), 6.97 (t, J=8.0 Hz, 1H), 6.80 (d, J=8.0 Hz, 1H), 4.16-4.05 (m, 1H), 2.39-2.34 (m, 2H), 2.30 (s, 3H), 2.19-2.11 (m, 2H), 2.01-1.97 (m, 1H), 1.89 (d, J=13.2 Hz, 2H), 1.55-1.50 (m, 2H), 1.21-1.08 (m, 2H).

### Step E. Procedure for Preparation of tert-butyl 4-((E)-3-((1s,4s)-4-(3-bromo-2-methylphenoxy)cyclohexyl)prop-1-en-1-yl)piperidine-1-carboxylate

To a solution of (1-tert-butoxycarbonyl-4-piperidyl)methyl-triphenyl-phosphonium; iodide (453.04 mg, 771.18 μmol, 1.2 equiv.) in THE (10 mL) was added LDA (2 M, 963.97 μL, 3 equiv.) at 0° C. The mixture was stirred for 2 hours at 0° C. 2-((1s,4s)-4-(3-bromo-2-methylphenoxy)cyclohexyl)acetaldehyde (200 mg, 642.65 μmol, 1 equiv.) in THE (5 mL) was added into the mixture at 0° C. The mixture was stirred at 25° C. for 12 hours. The reaction mixture was diluted with NH₄Cl (30 mL) and extracted with EtOAc (20 mL×3). The combined organic layers were washed with aqueous NaCl (30 mL×1), dried over Na₂SO₄, filtered, and concentrated under reduced pressure to give a residue. The residue was purified by reverse-phase HPLC (with 0.1% formic acid) to give tert-butyl 4-((E)-3-((1s,4s)-4-(3-bromo-2-methylphenoxy)cyclohexyl)prop-1-en-1-yl)piperidine-1-carboxylate (270 mg, 460.52 μmol, 35.83% yield) as a yellow oil.

MS (ESI) m/z: 436.3 [M−56+H]⁺

### Step F. Procedure for Preparation of tert-butyl 4-(3-((1s,4r)-4-(3-bromo-2-methylphenoxy)cyclohexyl)propyl)piperidine-1-carboxylate

To a solution of tert-butyl 4-((E)-3-((1s,4s)-4-(3-bromo-2-methylphenoxy)cyclohexyl)prop-1-en-1-yl)piperidine-1-carboxylate (270 mg, 548.24 μmol, 1 equiv.) in EtOH (10 mL) was added PtO₂ (99.59 mg, 438.59 μmol, 0.8 equiv.) under N₂ atmosphere. The suspension was degassed and purged with H₂ three times. The mixture was stirred under H₂ (15 Psi) at 25° C. for 1 hour. The reaction solution was filtered through the diatom layer under a moderate N₂ atmosphere to give tert-butyl 4-(3-((1s,4r)-4-(3-bromo-2-methylphenoxy)cyclohexyl)propyl)piperidine-1-carboxylate (250 mg, crude) as a yellow oil.

MS (ESI) m/z: 438.3 [M+H−56]⁺

### Step G. Procedure for Preparation of 4-(3-((1s,4r)-4-(3-bromo-2-methylphenoxy)cyclohexyl)propyl)piperidine

A mixture of tert-butyl 4-(3-((1s,4r)-4-(3-bromo-2-methylphenoxy)cyclohexyl)propyl)piperidine-1-carboxylate (250 mg, 505.56 μmol, 1 equiv.) in HCl/dioxane (10 mL) was degassed and purged with N₂ three times. The mixture was stirred at 25° C. for 2 hours under N₂ atmosphere. The reaction mixture was concentrated under reduced pressure to give a residue. The residue was purified by reverse-phase HPLC (with 0.1% formic acid) to give 4-(3-((1s,4r)-4-(3-bromo-2-methylphenoxy)cyclohexyl)propyl)piperidine (220 mg, crude) as a yellow oil.

MS (ESI) m/z: 394.3 [M+H]⁺

### Step H. Procedure for Preparation of 3-(6-iodo-1-methyl-indazol-3-yl)piperidine-2,6-dione

A solution of 3-(6-amino-1-methyl-indazol-3-yl)piperidine-2,6-dione (1 g, 3.87 mmol, 1 equiv.) in HCl (12 M, 1.61 mL, 5 equiv.) and H₂O (5 mL) was stirred for 30 min. The mixture was cooled to 0° C., and NaNO₂ (280.51 mg, 4.07 mmol, 1.05 equiv.) in H₂O (5 mL) was added. The mixture was stirred at 0° C. for 30 min. KI (1.29 g, 7.74 mmol, 2 equiv.) in H₂O (10 mL) was added to the mixture at 0° C. The mixture was stirred at 25° C. for 12 hours. The reaction mixture was quenched with H₂O (60 mL) and extracted with EtOAc (10 mL×3). The combined organic layers were washed with brine (20 mL×1), dried over Na₂SO₄, filtered, and concentrated under reduced pressure to give a residue. The residue was purified by flash silica gel chromatography (0˜40% ethyl acetate/petroleum ether) to give 3-(6-iodo-1-methyl-indazol-3-yl)piperidine-2,6-dione (400 mg, 975.20 μmol, 25.19% yield, 90% purity) as a yellow solid.

MS (ESI) m/z: 370.1 [M+H]⁺

### Step I. Procedure for Preparation of 3-(6-(4-(3-((1s,4r)-4-(3-bromo-2-methylphenoxy)cyclohexyl)propyl)piperidin-1-yl)-1-methyl-1H-indazol-3-yl)piperidine-2,6-dione

A mixture of 3-(6-iodo-1-methyl-indazol-3-yl)piperidine-2,6-dione (101.09 mg, 273.84 μmol, 1.2 equiv.), 4-(3-((1s,4r)-4-(3-bromo-2-methylphenoxy)cyclohexyl)propyl)piperidine-1-carboxylate (90 mg, 228.20 μmol, 1 equiv.), Cs₂CO₃ (223.06 mg, 684.61 μmol, 3 equiv.), and 1,3-bis[2,6-bis(1-propylbutyl)phenyl]-4,5-dichloro-2H-imidazol-1-ium-2-ide; 3-chloropyridine; dichloropalladium (44.40 mg, 45.64 μmol, 0.2 equiv.) in dioxane (4 mL) was degassed and purged with N₂ three times. The mixture was stirred at 90° C. for 5 hours under N₂ atmosphere in a microwave reactor. The reaction mixture was quenched with H₂O (20 mL) and extracted with EtOAc (10 mL×3). The combined organic layers were washed with brine (20 mL×1), dried over Na₂SO₄, filtered, and concentrated under reduced pressure to give a residue. The residue was purified by flash silica gel chromatography (0˜35% ethyl acetate/petroleum ether) to give 3-(6-(4-(3-((1s,4r)-4-(3-bromo-2-methylphenoxy)cyclohexyl)propyl)piperidin-1-yl)-1-methyl-1H-indazol-3-yl)piperidine-2,6-dione (20 mg, 27.06 μmol, 11.86% yield) as a yellow solid.

MS (ESI) m/z: 635.4 [M+H]⁺

### Step J. Procedure for Preparation of tert-butyl 6-(8-(benzo[d]thiazol-2-ylcarbamoyl)-3,4-dihydroisoquinolin-2(1H)-yl)-3-(3-(((1r,4s)-4-(3-(1-(3-(2,6-dioxopiperidin-3-yl)-1-methyl-1H-indazol-6-yl)piperidin-4-yl)propyl)cyclohexyl)oxy)-2-methylphenyl)picolinate

A mixture of 3-(6-(4-(3-((1s,4r)-4-(3-bromo-2-methylphenoxy)cyclohexyl)propyl)piperidin-1-yl)-1-methyl-1H-indazol-3-yl)piperidine-2,6-dione (20 mg, 31.46 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (23.13 mg, 37.76 μmol, 1.2 equiv.), KF (3.66 mg, 62.93 μmol, 1.47 μL, 2 equiv.), and Ad₂nBuP Pd G₃ (2.29 mg, 3.15 μmol, 0.1 equiv.) in dioxane (3 mL) and H₂O (0.5 mL) was degassed and purged with N₂ three times. The mixture was stirred at 100° C. for 1 hour under N₂ atmosphere in a microwave reactor. The reaction mixture was concentrated under reduced pressure to give a residue. The residue was purified by prep-TLC (SiO₂, petroleum ether:ethyl acetate=0.1) to give tert-butyl 6-(8-(benzo[d]thiazol-2-ylcarbamoyl)-3,4-dihydroisoquinolin-2(1H)-yl)-3-(3-(((1r,4s)-4-(3-(1-(3-(2,6-dioxopiperidin-3-yl)-1-methyl-1H-indazol-6-yl)piperidin-4-yl)propyl)cyclohexyl)oxy)-2-methylphenyl)picolinate (15 mg, 10.08 μmol, 32.05% yield) as a yellow oil.

MS (ESI) m/z: 1041.6 [M+H]⁺

### Step K. Procedure for Preparation of 6-(8-(benzo[d]thiazol-2-ylcarbamoyl)-3,4-dihydroisoquinolin-2(1H)-yl)-3-(3-(((1r,4s)-4-(3-(1-(3-(2,6-dioxopiperidin-3-yl)-1-methyl-1H-indazol-6-yl)piperidin-4-yl)propyl)cyclohexyl)oxy)-2-methylphenyl) picolinic acid

A solution of tert-butyl 6-(8-(benzo[d]thiazol-2-ylcarbamoyl)-3,4-dihydroisoquinolin-2(1H)-yl)-3-(3-(((1r,4s)-4-(3-(1-(3-(2,6-dioxopiperidin-3-yl)-1-methyl-1H-indazol-6-yl)piperidin-4-yl)propyl)cyclohexyl)oxy)-2-methylphenyl)picolinate (15 mg, 14.41 μmol, 1 equiv.) in DCM (2 mL) and TFA (2 mL) was degassed and purged with N₂ three times. The mixture was stirred at 25° C. for 12 hours under N₂ atmosphere. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1r,4s)-4-(3-(1-(3-(2,6-dioxopiperidin-3-yl)-1-methyl-1H-indazol-6-yl)piperidin-4-yl)propyl)cyclohexyl)oxy)-2-methylphenyl) picolinic acid (12.54 mg, 11.97 μmol, 83.09% yield) as a yellow solid.

MS (ESI) m/z: 985.8 [M+H]⁺

¹H NMR (400 MHz, DMSO-d₆) δ=12.91-12.82 (m, 1H), 12.67-12.44 (m, 1H), 10.84 (s, 1H), 8.03 (d, J=7.6 Hz, 1H), 7.79 (d, J=8.0 Hz, 1H), 7.65-7.59 (m, 1H), 7.51-7.42 (m, 4H), 7.41-7.30 (m, 2H), 7.11-7.02 (m, 1H), 6.96 (d, J=8.8 Hz, 1H), 6.93-6.83 (m, 3H), 6.60 (d, J=7.6 Hz, 1H), 4.98 (s, 2H), 4.61 (s, 1H), 4.30-4.21 (m, 1H), 3.96-3.85 (m, 5H), 3.80-3.71 (m, 2H), 3.05-3.00 (m, 2H), 2.75-2.58 (m, 4H), 2.20-2.11 (m, 2H), 1.94-1.87 (m, 5H), 1.78-1.72 (m, 2H), 1.56-1.47 (m, 4H), 1.34-1.23 (m, 12H)

### Example 255. Preparation of Compound 313

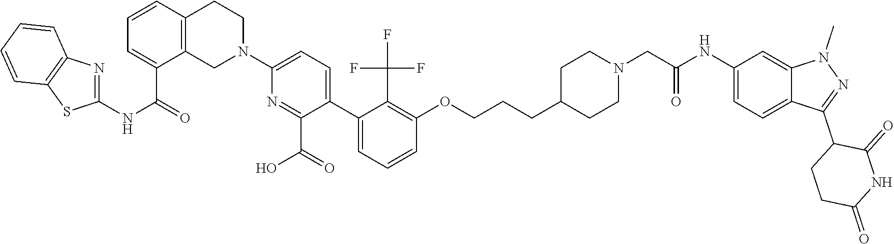 [SMILES: Cn1nc(C2CCC(=O)NC2=O)c2ccc(NC(=O)CN3CCC(CCCOc4cccc(-c5ccc(N6CCc7cccc(C(=O)Nc8nc9ccccc9s8)c7C6)nc5C(=O)O)c4C(F)(F)F)CC3)cc21]

### 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-2-oxo-ethyl]-4-piperidyl]propoxy]-2-(trifluoromethyl)phenyl]pyridine-2-carboxylic acid

### Step A. Procedure for Preparation of tert-butyl 4-[3-[3-bromo-2-(trifluoromethyl)phenoxy]propyl]piperidine-1-carboxylate

To a solution of 1-bromo-3-fluoro-2-(trifluoromethyl)benzene (500 mg, 2.06 mmol, 1 equiv.) in MeCN (6 mL) was added t-BuOK (692.68 mg, 6.17 mmol, 3 equiv.) and tert-butyl 4-(3-hydroxypropyl)piperidine-1-carboxylate (500.71 mg, 2.06 mmol, 1 equiv.). The mixture was stirred at 60° C. for 12 hours. The reaction was concentrated under reduced pressure to give a residue. The residue was purified by flash silica gel chromatography (0˜7% ethyl acetate/petroleum ether) to give tert-butyl 4-[3-[3-bromo-2-(trifluoromethyl)phenoxy]propyl]piperidine-1-carboxylate (857 mg, 1.82 mmol, 88.66% yield) as a white solid.

MS (ESI) m/z: 412.2 [M+H]⁺.

¹H NMR (400 MHz, DMSO-d₆) δ=7.50-7.44 (m, 1H), 7.38 (d, J=8.0 Hz, 1H), 7.27 (d, J=8.4 Hz, 1H), 4.08 (t, J=6.4 Hz, 2H), 3.91 (d, J=12.0 Hz, 2H), 2.75-2.57 (m, 2H), 1.78-1.69 (m, 2H), 1.63 (d, J=11.6 Hz, 2H), 1.38 (s, 9H), 1.37-1.30 (m, 3H), 1.01-0.88 (m, 2H).

### Step B. Procedure for Preparation of 4-[3-[3-bromo-2-(trifluoromethyl)phenoxy]propyl]piperidine

A solution of tert-butyl 4-[3-[3-bromo-2-(trifluoromethyl)phenoxy]propyl]piperidine-1-carboxylate (857 mg, 1.84 mmol, 1 equiv.) in HCl/dioxane (10 mL) was stirred at 25° C. for 12 hours. The reaction was concentrated under reduced pressure to give 4-[3-[3-bromo-2-(trifluoromethyl)phenoxy]propyl]piperidine (730 mg, 1.80 mmol, 97.9% yield) as a yellow solid.

MS (ESI) m/z: 366.2 [M+H]⁺.

¹H NMR (400 MHz, DMSO-d₆) δ=9.34-8.36 (m, 2H), 7.52-7.44 (m, 1H), 7.38 (d, J=8.0 Hz, 1H), 7.28 (d, J=8.4 Hz, 1H), 4.10 (t, J=6.0 Hz, 2H), 3.22 (d, J=10.8 Hz, 2H), 2.89-2.71 (m, 2H), 1.83-1.68 (m, 4H), 1.63-1.47 (m, 1H), 1.41-1.23 (m, 4H).

### Step C. Procedure for Preparation of ethyl 2-[4-[3-[3-bromo-2-(trifluoromethyl)phenoxy]propyl]-1-piperidyl]acetate

To a solution of 4-[3-[3-bromo-2-(trifluoromethyl)phenoxy]propyl]piperidine (730 mg, 1.81 mmol, 1 equiv., HCl) in MeCN (8 mL) was added K₂CO₃ (751.65 mg, 5.44 mmol, 3 equiv.) and ethyl 2-bromoacetate (272.48 mg, 1.63 mmol, 180.57 μL, 0.9 equiv.). The mixture was stirred at 60° C. for 1 hour. The reaction was concentrated under reduced pressure to give a residue. The residue was purified by flash silica gel chromatography (0˜45% ethyl acetate/petroleum ether) to give ethyl 2-[4-[3-[3-bromo-2-(trifluoromethyl)phenoxy]propyl]-1-piperidyl]acetate (650 mg, 1.42 mmol, 78.07% yield, 98.48% purity) as a yellow solid.

MS (ESI) m/z: 452.2 [M+H]⁺.

¹H NMR (400 MHz, DMSO-d₆) δ=7.50-7.44 (m, 1H), 7.37 (d, J=8.0 Hz, 1H), 7.27 (d, J=8.4 Hz, 1H), 4.06 (q, J=7.2 Hz, 4H), 3.15 (s, 2H), 2.79 (d, J=11.6 Hz, 2H), 2.15-2.05 (m, 2H), 1.76-1.68 (m, 2H), 1.61 (d, J=11.2 Hz, 2H), 1.37-1.29 (m, 2H), 1.18 (t, J=7.2 Hz, 4H), 1.11 (dd, J=3.6, 12.0 Hz, 2H).

### Step D. Procedure for Preparation of tert-butyl 6-[8-(1,3-benzothiazol-2-ylcarbamoyl)-3,4-dihydro-1H-isoquinolin-2-yl]-3-[3-[3-[1-(2-ethoxy-2-oxo-ethyl)-4-piperidyl]propoxy]-2-(trifluoromethyl)phenyl]pyridine-2-carboxylate

Ethyl 2-[4-[3-[3-bromo-2-(trifluoromethyl)phenoxy]propyl]-1-piperidyl]acetate (200 mg, 442.18 mol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (297.94 mg, 486.40 mol, 1.1 equiv.), Ad₂nBuP Pd G₃ (64.41 mg, 88.44 μmol, 0.2 equiv.), and KF (1.5 M, 884.36 μL, 3 equiv.) were taken up into a microwave tube in dioxane (5 mL), and the tube was sealed. The sealed tube was heated at 100° C. for 1 hour in a microwave reactor. The reaction was concentrated under reduced pressure to give a residue. The residue was purified by flash silica gel chromatography (0˜7% dichloromethane:methanol) to give tert-butyl 6-[8-(1,3-benzothiazol-2-ylcarbamoyl)-3,4-dihydro-1H-isoquinolin-2-yl]-3-[3-[3-[1-(2-ethoxy-2-oxo-ethyl)-4-piperidyl]propoxy]-2-(trifluoromethyl)phenyl]pyridine-2-carboxylate (310 mg, 275.54 μmol, 62.31% yield) as a brown oil.

MS (ESI) m/z: 430.0 [M/2+H]⁺.

¹H NMR (400 MHz, DMSO-d₆) δ=12.87 (s, 1H), 8.02 (d, J=7.6 Hz, 1H), 7.78 (d, J=8.0 Hz, 1H), 7.60 (d, J=8.0 Hz, 1H), 7.50-7.44 (m, 3H), 7.41-7.32 (m, 4H), 6.95 (d, J=8.8 Hz, 1H), 6.65 (d, J=7.6 Hz, 1H), 4.10-4.03 (m, 6H), 3.88 (t, J=6.0 Hz, 2H), 3.18-3.12 (m, 3H), 3.03 (t, J=5.6 Hz, 2H), 2.78 (d, J=8.4 Hz, 2H), 2.27-2.23 (m, 1H), 2.09 (s, 2H), 1.75-1.70 (m, 3H), 1.59 (s, 2H), 1.36-1.33 (m, 2H), 1.05-0.99 (m, 12H).

### Step E. Procedure for Preparation of 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-(trifluoromethyl)phenoxy]propyl]-1-piperidyl]acetic acid

To a solution of tert-butyl 6-[8-(1,3-benzothiazol-2-ylcarbamoyl)-3,4-dihydro-1H-isoquinolin-2-yl]-3-[3-[3-[1-(2-ethoxy-2-oxo-ethyl)-4-piperidyl]propoxy]-2-(trifluoromethyl)phenyl]pyridine-2-carboxylate (310 mg, 361.31 μmol, 1 equiv.) in THE (6 mL) and H₂O (2 mL) was added LiOH·H₂O (75.81 mg, 1.81 mmol, 5 equiv.). The mixture was stirred at 25° C. for 12 hours. The reaction was concentrated under reduced pressure and diluted with HCl (1M). The pH of the reaction mixture was adjusted to 2-3. The resulting solid was filtered and dried. The solid was purified by prep-TLC (SiO₂, DCM:MeOH=8:1) to give 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-(trifluoromethyl)phenoxy]propyl]-1-piperidyl]acetic acid (140 mg, 145.71 μmol, 40.33% yield) as a yellow solid.

MS (ESI) m/z: 830.6 [M+H]⁺.

### Step F. Procedure for Preparation of tert-butyl 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-2-oxo-ethyl]-4-piperidyl]propoxy]-2-(trifluoromethyl)phenyl]pyridine-2-carboxylate

A solution of 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-(trifluoromethyl)phenoxy]propyl]-1-piperidyl]acetic acid (75 mg, 90.37 μmol, 1 equiv.) and 3-(6-amino-1-methyl-indazol-3-yl)piperidine-2,6-dione (70.02 mg, 271.11 μmol, 3 equiv.) in pyridine (1 mL) was stirred at 40° C. for 12 hours. To this mixture was added EDCI (25.99 mg, 135.55 μmol, 1.5 equiv.). The resulting mixture was stirred at 40° C. for 12 hours. The reaction mixture was diluted with water (5 mL) and extracted with DCM (5 mL×3). The combined organic layers were dried over Na₂SO₄, filtered, and concentrated under reduced pressure to give a residue. The residue was purified by reversed-phase HPLC (with 0.1% formic acid) to give tert-butyl 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-2-oxo-ethyl]-4-piperidyl]propoxy]-2-(trifluoromethyl)phenyl]pyridine-2-carboxylate (45 mg, 40.67 mol, 45.0% yield) as a white solid.

MS (ESI) m/z: 1070.4 [M+H]⁺.

### Step G. Procedure for Preparation of 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-2-oxo-ethyl]-4-piperidyl]propoxy]-2-(trifluoromethyl)phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-2-oxo-ethyl]-4-piperidyl]propoxy]-2-(trifluoromethyl)phenyl]pyridine-2-carboxylate (40 mg, 37.38 μmol, 1 equiv.) in TFA (1 mL) and DCM (1 mL) was stirred at 25° C. for 12 hours. The solution was concentrated under reduced pressure to give a residue. The residue was purified by reverse-phase HPLC to give 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-2-oxo-ethyl]-4-piperidyl]propoxy]-2-(trifluoromethyl)phenyl]pyridine-2-carboxylic acid (31.12 mg, 30.06 μmol, 80.4% yield) as a yellow solid.

MS (ESI) m/z: 1015.4 [M/2+H]⁺.

¹H NMR (400 MHz, DMSO-d6) δ=10.87 (s, 1H), 9.82 (s, 1H), 8.08-7.98 (m, 2H), 7.78 (d, J=8.0 Hz, 1H), 7.62 (d, J=8.4 Hz, 2H), 7.50-7.43 (m, 3H), 7.40-7.31 (m, 3H), 7.25-7.15 (m, 2H), 7.00-6.93 (m, 1H), 6.68 (d, J=7.6 Hz, 1H), 5.06-4.92 (m, 2H), 4.32 (d, J=5.2, 9.6 Hz, 1H), 4.09 (t, J=5.6 Hz, 2H), 3.97-3.88 (m, 5H), 3.12 (s, 2H), 3.02 (s, 2H), 2.90-2.84 (m, 4H), 2.20-2.13 (m, 4H), 1.79-1.73 (m, 2H), 1.67 (d, J=7.2 Hz, 2H), 1.44-1.37 (m, 2H), 1.32-1.24 (m, 3H)

### Example 256. Preparation of Compound 314

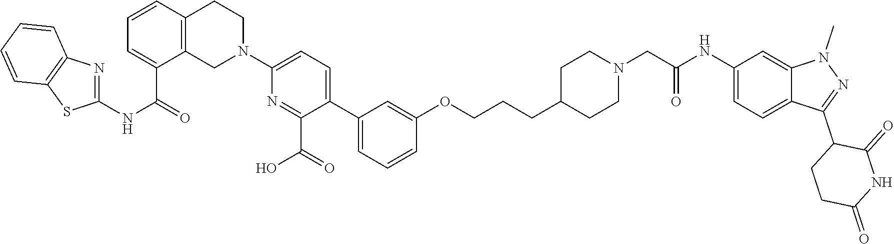 [SMILES: Cn1nc(C2CCC(=O)NC2=O)c2ccc(NC(=O)CN3CCC(CCCOc4cccc(-c5ccc(N6CCc7cccc(C(=O)Nc8nc9ccccc9s8)c7C6)nc5C(=O)O)c4)CC3)cc21]

### 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-6-yl)amino)-2-oxoethyl)piperidin-4-yl)propoxy)phenyl)picolinic acid

### Step A. Procedure for Preparation of tert-butyl 4-(3-(3-bromophenoxy)propyl)piperidine-1-carboxylate

A mixture of 3-bromophenol (500 mg, 2.89 mmol, 1 equiv.), tert-butyl 4-(3-hydroxypropyl)piperidine-1-carboxylate (703.27 mg, 2.89 mmol, 1 equiv.), and 2-(tributyl-phosphanylidene)acetonitrile (837.03 mg, 3.47 mmol, 1.2 equiv.) in toluene (6 mL) was degassed and purged with N₂ three times. The mixture was stirred at 120° C. for 16 hours under N₂ atmosphere. The reaction mixture was filtered and concentrated under reduced pressure to give a residue. The residue was purified by flash silica gel chromatography (0˜20% ethyl acetate/petroleum ether) to give tert-butyl 4-(3-(3-bromophenoxy)propyl)piperidine-1-carboxylate (850 mg, 2.13 mmol, 73.8% yield) as a white oil.

MS (ESI) m/z: 342.1 [M+H]⁺.

### Step B. Procedure for Preparation of 4-(3-(3-bromophenoxy)propyl)piperidine

A mixture of tert-butyl 4-(3-(3-bromophenoxy)propyl)piperidine-1-carboxylate (850 mg, 2.13 mmol, 1 equiv.) in TFA (4 mL) and DCM (4 mL) was stirred at 40° C. for 1 hour. The reaction mixture was filtered and concentrated under reduced pressure to give 4-(3-(3-bromophenoxy)propyl)piperidine (630 mg, 2.11 mmol, 99.0% yield) as a yellow oil.

MS (ESI) m/z: 298.1 [M+H]⁺.

### Step C. Procedure for Preparation of ethyl 2-(4-(3-(3-bromophenoxy)propyl)piperidin-1-yl)acetate

A mixture of 4-(3-(3-bromophenoxy)propyl)piperidine (630 mg, 2.11 mmol, 1 equiv.), ethyl 2-bromoacetate (352.80 mg, 2.11 mmol, 233.80 μL, 1 equiv.), and K₂CO₃ (1.46 g, 10.56 mmol, 5 equiv.) in MeCN (8 mL) was stirred at 60° C. for 2 hours. The combined organic layers were filtered and concentrated under reduced pressure to give a residue. The residue was purified by flash silica gel chromatography (0˜40% ethyl acetate/petroleum ether) to give ethyl 2-(4-(3-(3-bromophenoxy)propyl)piperidin-1-yl)acetate (720 mg, 1.87 mmol, 88.6% yield) as a yellow oil.

MS (ESI) m/z: 384.1 [M+H]⁺.

### Step D. Procedure for Preparation of tert-butyl 6-(8-(benzo[d]thiazol-2-ylcarbamoyl)-3, 4-dihydroisoquinolin-2(1H)-yl)-3-(3-(3-(1-(2-ethoxy-2-oxoethyl) piperidin-4-yl)propoxy)phenyl)picolinate

A mixture of ethyl 2-(4-(3-(3-bromophenoxy)propyl)piperidin-1-yl)acetate (150 mg, 390.31 μmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl) picolinate (286.90 mg, 468.38 μmol, 1.2 equiv.), K₂CO₃ (1.5 M, 390.31 μL, 1.5 eq), and [2-(2-aminophenyl)phenyl]palladium(1⁺); bis(1-adamantyl)-butyl-phosphane; methanesulfonate (56.85 mg, 78.06 μmol, 0.2 equiv.) in dioxane (2 mL) was stirred at 100° C. for 1.5 hours in a microwave reactor. The combined organic layers were filtered and concentrated under reduced pressure to give a residue. The residue was purified by flash silica gel chromatography (0˜40% ethyl acetate/petroleum ether) to give tert-butyl 6-(8-(benzo[d]thiazol-2-ylcarbamoyl)-3, 4-dihydroisoquinolin-2(1H)-yl)-3-(3-(3-(1-(2-ethoxy-2-oxoethyl) piperidin-4-yl)propoxy)phenyl)picolinate (200 mg, 253.17 μmol, 64.6% yield) as a yellow solid.

MS (ESI) m/z: 790.6 [M+H]⁺.

### Step E. Procedure for Preparation of 2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)phenoxy)propyl)piperidin-1-yl)acetic acid

A mixture of tert-butyl 6-(8-(benzo[d]thiazol-2-ylcarbamoyl)-3, 4-dihydroisoquinolin-2(1H)-yl)-3-(3-(3-(1-(2-ethoxy-2-oxoethyl) piperidin-4-yl)propoxy)phenyl)picolinate (200 mg, 253.17 μmol, 1 equiv.) and LiOH H₂O (53.12 mg, 1.27 mmol, 5 equiv.) in THF (4 mL) and H₂O (2 mL) was stirred at 40° C. for 2 hours. The reaction mixture was diluted with H₂O (5 mL) and concentrated to provide a turbid liquid. Then the pH of the turbid liquid was adjusted to ˜3 with HCl (1 M, 5 mL). The resulting mixture was filtered, dried, and extracted with DCM/MeOH (10:1). The organic layers were combined and concentrated under reduced pressure to give 2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)phenoxy)propyl)piperidin-1-yl)acetic acid (180 mg, 236.24 μmol, 93.31% yield) as a yellow solid.

MS (ESI) m/z: 762.6 [M+H]⁺.

### Step F. Procedure for Preparation of tert-butyl 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-6-yl)amino)-2-oxoethyl)piperidin-4-yl)propoxy)phenyl)picolinate

A mixture of 2-(4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)phenoxy)propyl)piperidin-1-yl)acetic acid (100 mg, 131.25 mol, 1 equiv.), 3-(6-amino-1-methyl-1H-indazol-3-yl)piperidine-2,6-dione (40.68 mg, 157.50 mol, 1.2 equiv.), and EDCI (32.71 mg, 170.62 μmol, 1.3 equiv.) in pyridine (1 mL) was stirred at 40° C. for 2 hours. The combined organic layers were filtered and concentrated under reduced pressure to give a residue. The residue was purified by flash silica gel chromatography (0˜10% ethyl acetate/petroleum ether) to give tert-butyl 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-6-yl)amino)-2-oxoethyl)piperidin-4-yl)propoxy)phenyl)picolinate (80 mg, 79.83 μmol, 60.82% yield) as a black solid.

MS (ESI) m/z: 1002.7 [M+H]⁺.

### Step G. Procedure for Preparation of 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-6-yl)amino)-2-oxoethyl)piperidin-4-yl)propoxy)phenyl)picolinic acid

A mixture of tert-butyl 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-6-yl)amino)-2-oxoethyl)piperidin-4-yl)propoxy)phenyl)picolinate (80 mg, 79.83 μmol, 1 equiv.) in TFA (0.6 mL) and DCM (0.6 mL) was stirred at 40° C. for 2 hours. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(3-(1-(2-((3-(2,6-dioxopiperidin-3-yl)-1-methyl-1H-indazol-6-yl)amino)-2-oxoethyl)piperidin-4-yl)propoxy)phenyl)picolinic acid (30.38 mg, 31.68 μmol, 39.69% yield) as a white solid.

MS (ESI) m/z: 946.6 [M+H]⁺.

¹H NMR (400 MHz, DMSO-d₆) δ=13.07-12.74 (m, 1H), 10.88 (s, 1H), 9.85 (s, 1H), 8.14 (s, 1H), 8.07-8.01 (m, 2H), 7.80 (d, J=7.6 Hz, 1H), 7.68-7.61 (m, 3H), 7.50-7.43 (m, 2H), 7.39-7.33 (m, 2H), 7.27 (t, J=8.4 Hz, 1H), 7.22 (d, J=7.6 Hz, 1H), 6.96 (d, J=9.2 Hz, 1H), 6.91-6.85 (m, 3H), 4.98 (s, 2H), 4.33 (dd, J=5.2, 10.0 Hz, 1H), 3.98-3.94 (m, 2H), 3.93-3.88 (m, 5H), 3.17-3.14 (m, 2H), 3.02 (t, J=5.2 Hz, 2H), 2.89 (d, J=10.0 Hz, 2H), 2.68-2.63 (m, 2H), 2.33 (s, 3H), 2.20-2.16 (m, 2H), 1.78-1.67 (m, 4H), 1.40-1.34 (m, 2H), 1.29 (s, 2H)

### Example 258. Preparation of Compound 316a

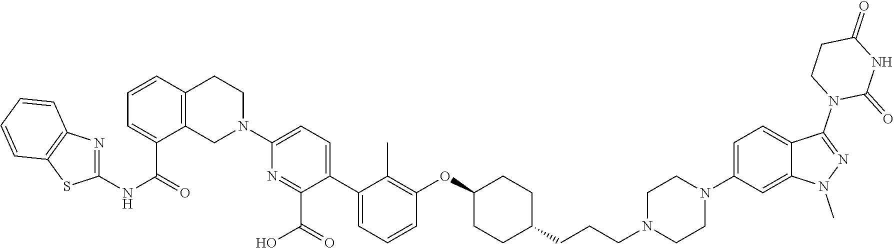 [SMILES: Cc1c(O[C@H]2CC[C@H](CCCN3CCN(c4ccc5c(N6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(3-(4-(3-(2,4-dioxotetrahydropyrimidin-1(2H)-yl)-1-methyl-1H-indazol-6-yl)piperazin-1-yl)propyl)cyclohexyl)oxy)-2-methylphenyl)picolinic acid

### Step A. Procedure for Preparation of ethyl (E)-3-((1r,4r)-4-(3-bromo-2-methylphenoxy)cyclohexyl)acrylate

To a mixture of NaH (2.22 g, 55.52 mmol, 60% purity, 1.5 equiv.) in THE (300 mL) was added dropwise ethyl 2-diethoxyphosphorylacetate (11.62 g, 51.82 mmol, 10.28 mL, 1.4 equiv.) at 0° C. under N₂. The mixture was stirred at 0° C. for 1 hour under N₂. To this mixture was added (1r,4r)-4-(3-bromo-2-methylphenoxy)cyclohexane-1-carbaldehyde (11 g, 37.01 mmol, 1.0 equiv.). The resulting reaction mixture was stirred at 25° C. for 2 hours under N₂ atmosphere. The reaction mixture was quenched with aqueous NH₄Cl (250 mL) at 0° C. and extracted with ethyl acetate (250 mL×2). The combined organic layers were washed with brine (250 mL×2), dried over Na₂SO₄, filtered, and concentrated under reduced pressure to give a residue. The residue was purified by flash silica gel chromatography (0-10% ethyl acetate/petroleum ether) to give compound ethyl (E)-3-((1r,4r)-4-(3-bromo-2-methylphenoxy)cyclohexyl)acrylate (24 g, 65.35 mmol, 88.2% yield) as a white solid.

¹H NMR (400 MHz, DMSO-d₆) δ=7.18-7.12 (m, 1H), 7.10-7.03 (m, 2H), 6.85 (m, 1H), 5.90-5.72 (m, 1H), 4.31-4.22 (m, 1H), 4.11 (q, J=7.2 Hz, 2H), 2.30-2.23 (m, 1H), 2.21 (s, 3H), 2.08 (m, 2H), 1.81 (m, 2H), 1.51-1.25 (m, 4H), 1.21 (t, J=7.2 Hz, 3H).

### Step B. Procedure for Preparation of ethyl 3-((1r,4r)-4-(3-bromo-2-methylphenoxy)cyclohexyl)propanoate

A mixture of ethyl (E)-3-((1r,4r)-4-(3-bromo-2-methylphenoxy)cyclohexyl)acrylate (12 g, 32.67 mmol, 1.0 equiv.) and PtO₂ (2.97 g, 13.07 mmol, 0.4 equiv.) in EtOH (200 mL) was degassed and purged with N₂ three times. The mixture was stirred at 25° C. for 16 hours under H₂ (15 Psi) atmosphere. The mixture was filtered washed with ethyl acetate (100 mL). The filtrate was concentrated under reduced pressure to give ethyl 3-((1r,4r)-4-(3-bromo-2-methylphenoxy)cyclohexyl)propanoate (24 g, 64.99 mmol, 99.4% yield) as a white solid.

¹H NMR (400 MHz, DMSO-d₆) δ=7.15-7.11 (m, 1H), 7.06 (t, J=8.0 Hz, 1H), 7.02-6.98 (m, 1H), 4.26-4.16 (m, 1H), 4.04 (q, J=7.2 Hz, 2H), 2.32-2.24 (m, 2H), 2.20 (s, 3H), 2.03 (m, 2H), 1.74 (m, 2H), 1.45 (q, J=7.2 Hz, 2H), 1.37-1.23 (m, 3H), 1.17 (t, J=7.2 Hz, 3H), 1.08-1.01 (m, 2H).

### Step C. Procedure for Preparation of 3-((1r,4s)-4-(3-bromo-2-methylphenoxy)cyclohexyl)propan-1-ol

To a solution of ethyl 3-((1r,4r)-4-(3-bromo-2-methylphenoxy)cyclohexyl)propanoate (12 g, 32.49 mmol, 1.0 equiv.) in THF (150 mL) was added LiAlH₄ (1.23 g, 32.49 mmol, 1.0 equiv.). The mixture was degassed and purged with N₂ three times. The reaction mixture was then stirred at 0° C. for 2 hours under N₂ atmosphere. The reaction mixture was quenched by the addition of Na₂SO₄·10H₂O (20 g) at 0° C. under a N₂ atmosphere, filtered, and concentrated under reduced pressure to give 3-((1r,4s)-4-(3-bromo-2-methylphenoxy)cyclohexyl)propan-1-ol (20.8 g, 63.56 mmol, 97.8% yield) as a yellow oil.

¹H NMR (400 MHz, DMSO-d₆) δ=7.15-7.11 (m, 1H), 7.06 (t, J=8.0 Hz, 1H), 7.03-6.98 (m, 1H), 4.34 (m, 1H), 4.26-4.17 (m, 1H), 3.37 (m, 2H), 2.20 (s, 3H), 2.08-1.99 (m, 2H), 1.81-1.71 (m, 2H), 1.45-1.39 (m, 2H), 1.38-1.29 (m, 2H), 1.20 (m, 7.2 Hz, 3H), 1.08-0.96 (m, 2H)

### Step D. Procedure for Preparation of 3-((1r,4r)-4-(3-bromo-2-methylphenoxy)cyclohexyl)propanal

A mixture of 3-((1r,4s)-4-(3-bromo-2-methylphenoxy)cyclohexyl)propan-1-ol (50 mg, 152.79 mol, 1.0 equiv.) and DMP (77.76 mg, 183.34 μmol, 56.80 μL, 1.2 equiv.) in DCM (1 mL) was degassed and purged with N₂ three times. The mixture was stirred at 25° C. for 20 hours under N₂ atmosphere. The mixture was filtered and washed with DCM (20 mL). The filtrate was concentrated under reduced pressure to give a residue. The residue was purified by prep-TLC (SiO₂, petroleum ether: ethyl acetate=3:1) to give 3-((1r,4r)-4-(3-bromo-2-methylphenoxy)cyclohexyl)propanal (30 mg, 92.24 μmol, 60.3% yield) as a colorless oil.

¹H NMR (400 MHz, DMSO-d₆) δ=9.67 (s, 1H), 7.16-7.11 (m, 1H), 7.09-6.99 (m, 2H), 4.29-4.16 (m, 1H), 2.47-2.41 (m, 2H), 2.20 (s, 3H), 2.04 (m, 2H), 1.75 (m, 2H), 1.45 (q, J=7.2 Hz, 2H), 1.38-1.23 (m, 3H), 1.10-0.98 (m, 2H).

### Step E. Procedure for Preparation of 1-(6-(4-(3-((1r,4s)-4-(3-bromo-2-methylphenoxy)cyclohexyl)propyl)piperazin-1-yl)-1-methyl-1H-indazol-3-yl)dihydropyrimidine-2,4(1H,3H)-dione

A solution of 3-((1r,4r)-4-(3-bromo-2-methylphenoxy)cyclohexyl)propanal (200 mg, 614.93 mol, 1.0 equiv.), 1-(1-methyl-6-piperazin-1-yl-indazol-3-yl)hexahydropyrimidine-2,4-dione (288.46 mg, 737.92 μmol, 1.2 equiv.), and KOAc (60.35 mg, 614.93 μmol, 1.0 equiv.) in DCM (6 mL) was stirred at 25° C. for 1 hour. To this solution was added NaBH(OAc)₃ (390.99 mg, 1.84 mmol, 3.0 equiv.), and the resulting mixture was stirred at 25° C. for 2 hours. After completion, the reaction mixture was poured into water (20 mL), and extracted with ethyl acetate (20 mL×3). The combined organic layers were washed with brine (20 mL), dried over anhydrous sodium sulfate, filtered, and concentrated under reduced pressure to give a residue. The residue was purified by prep-TLC (SiO₂, DCM:MeOH=10:1) to give 1-(6-(4-(3-((1r,4s)-4-(3-bromo-2-methylphenoxy)cyclohexyl)propyl)piperazin-1-yl)-1-methyl-1H-indazol-3-yl)dihydropyrimidine-2,4(1H,3H)-dione (120 mg, 166.28 μmol, 27.04% yield) as a yellow solid.

MS (ESI) m/z: 637.4 [M+H]⁺.

### Step F. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,4-dioxotetrahydropyrimidin-1(2H)-yl)-1-methyl-1H-indazol-6-yl)piperazin-1-yl)propyl)cyclohexyl)oxy)-2-methylphenyl)picolinate

A mixture of 1-(6-(4-(3-((1r,4s)-4-(3-bromo-2-methylphenoxy)cyclohexyl)propyl)piperazin-1-yl)-1-methyl-1H-indazol-3-yl)dihydropyrimidine-2,4(1H,3H)-dione (120 mg, 188.20 μmol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (172.92 mg, 282.30 μmol, 1.5 equiv.), Ad2nBuP Pd G₃ (13.71 mg, 18.82 μmol, 0.1 equiv.), and KF (32.80 mg, 564.61 μmol, 13.23 μL, 3.0 equiv.) in dioxane (4 mL) and H₂O (0.4 mL) was degassed and purged with N₂ three times. The mixture was stirred at 100° C. for 2 hours under N₂ atmosphere in a microwave reactor. After completion, the reaction mixture was poured into water (50 mL) and extracted with DCM (50 mL×3). The combined organic layers were washed with brine (50 mL), dried over anhydrous sodium sulfate, filtered, and concentrated under reduced pressure to give a residue. The residue was purified by prep-TLC (SiO₂, DCM:MeOH=10:1) to give tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,4-dioxotetrahydropyrimidin-1(2H)-yl)-1-methyl-1H-indazol-6-yl)piperazin-1-yl)propyl)cyclohexyl)oxy)-2-methylphenyl)picolinate (110 mg, 99.80 μmol, 53.03% yield) as a yellow solid.

MS (ESI) m/z: 1043.8 [M+H]⁺.

### Step G. Procedure for Preparation of 6-(8-(benzo[d]thiazol-2-ylcarbamoyl)-3,4-dihydroisoquinolin-2(1H)-yl)-3-(3-(((1s,4r)-4-(3-(4-(3-(2,4-dioxotetrahydropyrimidin-1(2H)-yl)-1-methyl-1H-indazol-6-yl)piperazin-1-yl)propyl)cyclohexyl)oxy)-2-methylphenyl)picolinic acid

A solution of tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,4-dioxotetrahydropyrimidin-1(2H)-yl)-1-methyl-1H-indazol-6-yl)piperazin-1-yl)propyl)cyclohexyl)oxy)-2-methylphenyl)picolinate (110 mg, 105.44 μmol, 1.0 equiv.) in DCM (1 mL) and TFA (2 mL) was stirred at 40° C. for 2 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-(3-(4-(3-(2,4-dioxotetrahydropyrimidin-1(2H)-yl)-1-methyl-1H-indazol-6-yl)piperazin-1-yl)propyl)cyclohexyl)oxy)-2-methylphenyl)picolinic acid (71.61 mg, 65.81 μmol, 62.42% yield) as a white solid.

MS (ESI) m/z: 987.7 [M+H]⁺.

¹H NMR (400 MHz, DMSO-d₆) δ=12.99-12.75 (m, 1H), 10.51 (s, 1H), 8.03 (d, J=8.0 Hz, 1H), 7.79 (m, 1H), 7.62 (m, 1H), 7.53-7.46 (m, 2H), 7.45 (m, 2H), 7.40-7.33 (m, 2H), 7.11-7.04 (m, 1H), 6.98-6.91 (m, 4H), 6.62 (m, 1H), 4.98 (m, 2H), 4.26-4.17 (m, 1H), 3.94-3.88 (m, 7H), 3.03 (m, 2H), 2.74 (m, 2H), 2.60 (m, 2H), 2.46-2.30 (m, 7H), 2.10 (m, 2H), 1.87 (s, 3H), 1.81 (m, J=10.4 Hz, 2H), 1.72-1.62 (m, 2H), 1.41-1.30 (m, 3H), 1.28-1.18 (m, 3H), 1.14-1.05 (m, 2H)

### Example 259. Preparation of Compound 328a

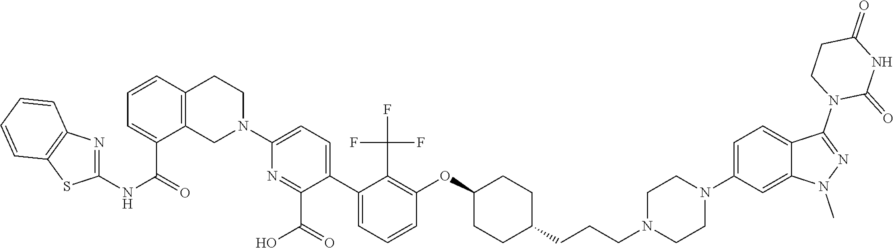 [SMILES: Cn1nc(N2CCC(=O)NC2=O)c2ccc(N3CCN(CCC[C@H]4CC[C@H](Oc5cccc(-c6ccc(N7CCc8cccc(C(=O)Nc9nc%10ccccc%10s9)c8C7)nc6C(=O)O)c5C(F)(F)F)CC4)CC3)cc21]

### 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)piperazin-1-yl)propyl)cyclohexyl)oxy)-2-(trifluoromethyl)phenyl)picolinic acid

### Step A. Procedure for Preparation of ethyl (E)-3-((1r,4r)-4-(3-bromo-2-(trifluoromethyl)phenoxy)cyclohexyl)acrylate

To a solution of (1r,4r)-4-(3-bromo-2-(trifluoromethyl)phenoxy)cyclohexane-1-carbaldehyde (368.69 mg, 1.64 mmol, 326.28 μL, 1.5 equiv.) in THE (20 mL) was added LiHMDS (1 M, 2.19 mL, 2 equiv.) at −10° C. The reaction mixture was stirred for 1 hour. Then (1r,4r)-4-(3-bromo-2-(trifluoromethyl)phenoxy)cyclohexane-1-carbaldehyde (385 mg, 1.10 mmol, 1 equiv.) was added, and the mixture was stirred at 0° C. for 1 hour under N₂ atmosphere. The reaction mixture was quenched with NH₄Cl (20 mL) at 0° C. and extracted with ethyl acetate (20 mL). The combined organic layers were washed with brine (20 mL), dried over sodium sulfate, filtered, and concentrated under reduced pressure to give a residue. The residue was purified by flash silica gel chromatography (0-4% ethyl acetate/petroleum ether) to give ethyl (E)-3-((1r,4r)-4-(3-bromo-2-(trifluoromethyl)phenoxy)cyclohexyl)acrylate (430 mg, 918.70 μmol, 83.52% yield) as a colorless oil.

¹H NMR (400 MHz, DMSO-d₆) δ=7.50-7.41 (m, 1H), 7.40-7.32 (m, 2H), 6.84 (dd, J=6.4, 16.0 Hz, 1H), 5.83 (d, J=16.0 Hz, 1H), 4.57-4.41 (m, 1H), 4.11 (q, J=7.2 Hz, 2H), 2.25 (d, J=6.8 Hz, 1H), 2.07 (d, J=10.0 Hz, 2H), 1.82 (d, J=12.0 Hz, 2H), 1.50-1.38 (m, 2H), 1.37-1.25 (m, 2H), 1.20 (t, J=7.2 Hz, 3H).

### Step B. Procedure for Preparation of ethyl 3-((1r,4r)-4-(3-bromo-2-(trifluoromethyl)phenoxy)cyclohexyl)propanoate

To a solution of ethyl (E)-3-((1r,4r)-4-(3-bromo-2-(trifluoromethyl)phenoxy)cyclohexyl)acrylate (430 mg, 1.02 mmol, 1 equiv.) in EtOH (4 mL) was added PtO₂ (23.18 mg, 102.08 μmol, 0.1 equiv.) under N₂ atmosphere. The suspension was degassed and purged with H₂ three times. The mixture was stirred under H₂ (15 Psi) at 25° C. for 12 hours. The reaction solution was filtered through the diatom layer under a moderate N₂ atmosphere. The residue was purified by flash silica gel chromatography (0-6% ethyl acetate/petroleum ether) to give ethyl 3-((1r,4r)-4-(3-bromo-2-(trifluoromethyl)phenoxy)cyclohexyl)propanoate (300 mg, 637.90 μmol, 62.49% yield) as a yellow oil.

¹H NMR (400 MHz, DMSO-d₆) δ=7.48-7.41 (m, 1H), 7.39-7.32 (m, 2H), 4.50-4.38 (m, 1H), 4.04 (q, J=7.2 Hz, 2H), 2.29 (t, J=7.6 Hz, 2H), 2.04 (d, J=10.0 Hz, 2H), 1.75 (d, J=12.0 Hz, 2H), 1.46 (q, J=7.2 Hz, 2H), 1.36-1.28 (m, 2H), 1.26-1.22 (m, 1H), 1.17 (t, J=7.2 Hz, 3H), 1.10-1.00 (m, 2H).

### Step C. Procedure for Preparation of 3-((1r,4s)-4-(3-bromo-2-(trifluoromethyl)phenoxy)cyclohexyl)propan-1-ol

To a solution of ethyl 3-((1r,4r)-4-(3-bromo-2-(trifluoromethyl)phenoxy)cyclohexyl)propanoate (150 mg, 354.39 μmol, 1 equiv.) in THF (10 mL) was added LiAlH₄ (13.45 mg, 354.39 μmol, 1 equiv.). The mixture was stirred at 0° C. for 1 hour. The reaction was quenched with saturated Na₂SO₄ (3 mL). The organic phase was concentrated under reduced pressure to give a residue. The residue was purified by flash silica gel chromatography (0-20% ethyl acetate/petroleum ether) to give 3-((1r,4s)-4-(3-bromo-2-(trifluoromethyl)phenoxy)cyclohexyl)propan-1-ol (50 mg, 131.16 mol, 37.01% yield) as a yellow oil.

¹H NMR (400 MHz, DMSO-d₆) δ=7.48-7.41 (m, 1H), 7.38-7.32 (m, 2H), 4.50-4.38 (m, 1H), 4.34 (t, J=5.2 Hz, 1H), 3.40-3.34 (m, 2H), 2.04 (d, J=10.0 Hz, 2H), 1.76 (d, J=12.4 Hz, 2H), 1.46-1.31 (m, 4H), 1.23-1.16 (m, 3H), 1.09-0.98 (m, 2H).

### Step D. Procedure for Preparation of 3-((1r,4r)-4-(3-bromo-2-(trifluoromethyl)phenoxy)cyclohexyl)propanal

A solution of (COCl)₂ (33.29 mg, 262.31 μmol, 22.96 μL, 2 equiv.) in DCM (5 mL) was cooled to −78° C., and DMSO (40.99 mg, 524.62 μmol, 40.99 μL, 4 equiv.) was added dropwise. The mixture was stirred at −70° C. for 2 hours under N₂ atmosphere. Then 3-((1r,4s)-4-(3-bromo-2-(trifluoromethyl)phenoxy)cyclohexyl)propan-1-ol (50 mg, 131.16 μmol, 1 equiv.) was added dropwise, and the mixture was stirred at −70° C. for 1 hour under N₂ atmosphere. TEA (79.63 mg, 786.93 μmol, 109.53 μL, 6 equiv.) was added dropwise to the mixture at −70° C., and the mixture was warmed to 25° C. and stirred at 25° C. for 1 hour under N₂ atmosphere. The reaction mixture was diluted with H₂O (20 mL) and extracted with DCM (20 mL×3). The combined organic layers were washed with brine (10 mL×3), dried over Na₂SO₄, filtered, and concentrated under reduced pressure to give 3-((1r,4r)-4-(3-bromo-2-(trifluoromethyl)phenoxy)cyclohexyl)propanal (50 mg, crude) as a yellow oil.

¹H NMR (400 MHz, DMSO-d₆) δ=9.67 (t, J=1.2 Hz, 1H), 7.48-7.41 (m, 1H), 7.39-7.33 (m, 2H), 4.50-4.38 (m, 1H), 2.45 (t, J=1.6, 7.6 Hz, 2H), 2.08-2.00 (m, 2H), 1.79-1.71 (m, 2H), 1.45 (q, J=7.2 Hz, 2H), 1.39-1.27 (m, 3H), 1.09-1.01 (m, 2H).

### Step E. Procedure for Preparation of 3-(6-(4-(3-((1r,4s)-4-(3-bromo-2-(trifluoromethyl)phenoxy)cyclohexyl)propyl)piperazin-1-yl)-1-methyl-1H-indazol-3-yl)piperidine-2,6-dione

To a solution of 3-((1r,4r)-4-(3-bromo-2-(trifluoromethyl)phenoxy)cyclohexyl)propanal (60 mg, 158.22 μmol, 1 equiv.) and 3-(1-methyl-6-(piperazin-1-yl)-1H-indazol-3-yl)piperidine-2,6-dione (69.08 mg, 189.87 μmol, 1.2 equiv., HCl) in DCM (3 mL) was added NaOAc (12.98 mg, 158.22 mol, 1 equiv.). The mixture was stirred at 0° C. for 1 hour. To this mixture was then added NaBH(OAc)₃ (167.67 mg, 791.12 μmol, 5 equiv.). The mixture was stirred at 0 to 25° C. for 11 hours. The reaction mixture was quenched by addition of H₂O (5 mL) at 25° C. and extracted with DCM (5 mL×3). The combined organic layers were washed with brine (5 mL×3), dried over Na₂SO₄, filtered, and concentrated under reduced pressure to give a residue. The residue was purified by flash silica gel chromatography (0-7% MeOH/DCM) to give 3-(6-(4-(3-((1r,4s)-4-(3-bromo-2-(trifluoromethyl)phenoxy)cyclohexyl)propyl)piperazin-1-yl)-1-methyl-1H-indazol-3-yl)piperidine-2,6-dione (110 mg, crude) as a yellow oil.

MS (ESI) m/z: 690.2 [M+H]⁺.

### Step F. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)piperazin-1-yl)propyl)cyclohexyl)oxy)-2-(trifluoromethyl)phenyl)picolinate

A mixture of 3-(6-(4-(3-((1r,4s)-4-(3-bromo-2-(trifluoromethyl)phenoxy)cyclohexyl)propyl)piperazin-1-yl)-1-methyl-1H-indazol-3-yl)piperidine-2,6-dione (110 mg, 159.28 μmol, 1 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (117.08 mg, 191.14 μmol, 1.2 equiv.), KF (27.76 mg, 477.85 μmol, 11.19 μL, 3 equiv.), and Ad₂nBuP Pd G₃ (23.20 mg, 31.86 μmol, 0.2 equiv.) in dioxane (2 mL) and H₂O (0.2 mL) was degassed and purged with N₂ three times. The mixture was stirred at 100° C. for 2 hours under N₂ atmosphere. The reaction mixture was filtered and concentrated under reduced pressure to give a residue. The residue was purified by prep-TLC (SiO₂, DCM:MeOH=10:1) to give tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)piperazin-1-yl)propyl)cyclohexyl)oxy)-2-(trifluoromethyl)phenyl)picolinate (110 mg, 89.36 μmol, 56.10% yield) as a yellow oil.

MS (ESI) m/z: 1096.6 [M+H]⁺.

### Step G. Procedure for Preparation of 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)piperazin-1-yl)propyl)cyclohexyl)oxy)-2-(trifluoromethyl)phenyl)picolinic acid

A solution of tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)piperazin-1-yl)propyl)cyclohexyl)oxy)-2-(trifluoromethyl)phenyl)picolinate (110 mg, 100.34 μmol, 1 equiv.) in TFA (1 mL) and DCM (2 mL) was stirred at 25° C. for 12 hours. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)piperazin-1-yl)propyl)cyclohexyl)oxy)-2-(trifluoromethyl)phenyl)picolinic acid (31.76 mg, 28.28 μmol, 28.19% yield) as a white solid.

MS (ESI) m/z: 1040.6 [M+H]⁺.

¹H NMR (400 MHz, DMSO-d₆) δ=13.15-12.42 (m, 1H), 10.85 (s, 1H), 8.14 (s, 1H), 8.03 (d, J=7.6 Hz, 1H), 7.79 (d, J=8.0 Hz, 1H), 7.62 (d, J=7.6 Hz, 1H), 7.52-7.32 (m, 7H), 7.25 (d, J=8.4 Hz, 1H), 7.01-6.88 (m, 2H), 6.85 (s, 1H), 6.64 (d, J=7.6 Hz, 1H), 5.06-4.91 (m, 2H), 4.43 (t, J=10.0 Hz, 1H), 4.26 (dd, J=4.8, 9.2 Hz, 1H), 3.93 (t, J=5.6 Hz, 2H), 3.89 (s, 3H), 3.25 (s, 4H), 3.02 (t, J=5.6 Hz, 2H), 2.70-2.56 (m, 6H), 2.42 (d, J=1.6 Hz, 2H), 2.35-2.24 (m, 2H), 2.19-2.13 (m, 1H), 2.12-2.04 (m, 2H), 1.87-1.75 (m, 2H), 1.52 (s, 2H), 1.42-1.32 (m, 2H), 1.25 (d, J=7.2 Hz, 2H), 1.08 (q, J=12.0 Hz, 2H).

### Example 260. Preparation of Compound 329a

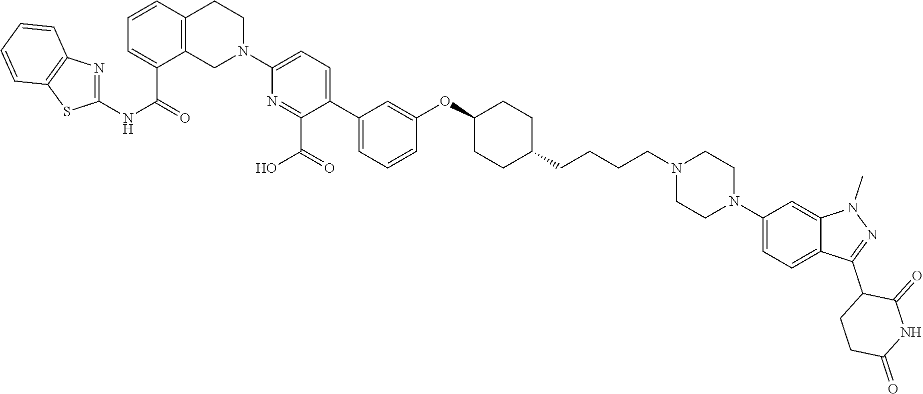 [SMILES: Cn1nc(C2CCC(=O)NC2=O)c2ccc(N3CCN(CCCC[C@H]4CC[C@H](Oc5cccc(-c6ccc(N7CCc8cccc(C(=O)Nc9nc%10ccccc%10s9)c8C7)nc6C(=O)O)c5)CC4)CC3)cc21]

### 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)butyl)cyclohexyl)oxy)phenyl)picolinic acid

### Step A. Procedure for Preparation of methyl (1r,4r)-4-(3-bromophenoxy)cyclohexane-1-carboxylate

To a solution of 3-bromophenol (2.5 g, 14.45 mmol, 1.0 equiv.) in toluene (25 mL) was added methyl 4-hydroxycyclohexanecarboxylate (2.29 g, 14.45 mmol, 1.0 equiv.) and 2-(tributyl-λ⁵-phosphanylidene)acetonitrile (4.19 g, 17.34 mmol, 1.2 equiv.) at 25° C. The reaction mixture was degassed and purged with N₂ three times. The mixture was stirred at 120° C. for 16 hours under N₂ atmosphere. The reaction mixture was filtered and concentrated under reduced pressure to give a residue. The residue was purified by flash silica gel chromatography (0˜5% ethyl acetate/petroleum ether) to give methyl (1r,4r)-4-(3-bromophenoxy)cyclohexane-1-carboxylate (6 g, 13.39 mmol, 46.34% yield) as a colorless oil.

¹H NMR (400 MHz, CDCl₃-d) δ=7.16-7.06 (m, 2H), 7.05 (s, 1H), 6.82 (d, J=8.4 Hz, 1H), 4.23-4.16 (m, 1H), 3.70 (s, 3H), 2.43-2.33 (m, 1H), 2.17 (d, J=12.4 Hz, 2H), 2.10 (s, 2H), 1.66-1.55 (m, 2H), 1.54-1.42 (m, 2H)

### Step B. Procedure for Preparation of ((1r,4r)-4-(3-bromophenoxy)cyclohexyl)methanol

To a solution of methyl (1r,4r)-4-(3-bromophenoxy)cyclohexane-1-carboxylate (3 g, 6.71 mmol, 1.0 equiv.) in THF (30 mL) was added LiAlH₄ (305.36 mg, 8.05 mmol, 1.2 equiv.) at 0° C. under N₂. The reaction mixture was stirred under N₂ at 0° C. for 1 hour. The reaction mixture was quenched with Na₂SO₄·10 H₂O (1 g) and slowly warmed to 20° C. The mixture was filtered, and the filter cake was washed with DCM (100 mL). The filtrate was concentrated under reduced pressure to give a residue. The residue was purified by flash silica gel chromatography (0˜20% ethyl acetate/petroleum ether) to give ((1r,4r)-4-(3-bromophenoxy)cyclohexyl)methanol (1.68 g, 5.68 mmol, 84.78% yield) as a yellow oil.

¹H NMR (400 MHz, CDCl₃-d) δ=7.15-7.10 (m, 1H), 7.08-7.04 (m, 2H), 6.85-6.80 (m, 1H), 4.20-4.10 (m, 1H), 3.51 (d, J=6.4 Hz, 2H), 2.24-2.14 (m, 2H), 1.98-1.88 (m, 2H), 1.63-1.52 (m, 1H), 1.51-1.40 (m, 3H), 1.18-1.05 (m, 2H)

### Step C. Procedure for Preparation of (1r,4r)-4-(3-bromophenoxy)cyclohexane-1-carbaldehyde

A solution of DMSO (1.84 g, 23.56 mmol, 1.84 mL, 4.0 equiv.) in DCM (20 mL) was added dropwise to a solution of oxalyl dichloride (1.50 g, 11.78 mmol, 1.03 mL, 2.0 equiv.) in DCM (5 mL) at −70° C. under a N₂ atmosphere. The mixture was stirred at −70° C. for 30 minutes. Afterwards, ((1r,4r)-4-(3-bromophenoxy)cyclohexyl)methanol (1.68 g, 5.89 mmol, 1.0 equiv.) in DCM (5 mL) was added dropwise at −70° C. The solution was stirred for 30 minutes at −70° C. Then, TEA (3.58 g, 35.35 mmol, 4.92 mL, 6.0 equiv.) was added into the solution. The solution was stirred at 25° C. for 1 hour under N₂ atmosphere. The reaction mixture was diluted with water (20 mL) and extracted with DCM (20 mL×3). The combined organic layers were washed with brine (10 mL×2), dried over Na₂SO₄, filtered, and concentrated under reduced pressure to give (1r,4r)-4-(3-bromophenoxy)cyclohexane-1-carbaldehyde (1.7 g, crude) as a yellow oil.

¹H NMR (400 MHz, DMSO-d₆) δ=9.60 (s, 1H), 7.26-7.18 (m, 1H), 7.15 (t, J=2.0 Hz, 1H), 7.12-7.06 (m, 1H), 6.96 (dd, J=1.6, 8.4 Hz, 1H), 4.41-4.30 (m, 1H), 2.38-2.29 (m, 1H), 2.08-1.92 (m, 4H), 1.48-1.36 (m, 4H)

### Step D. Procedure for Preparation of 1-(((1r,4r)-4-((E)-4-(benzyloxy)but-1-en-1-yl)cyclohexyl)oxy)-3-bromobenzene

A mixture of 3-benzyloxypropyl (triphenyl) phosphonium; bromide (3.10 g, 6.30 mmol, 1.05 equiv.) in THE (15 mL) was degassed and purged with N₂ three times. To this solution was added LiHMDS (1 M, 7.20 mL, 1.2 equiv.) dropwise. The reaction mixture was stirred at −70° C. for 2 hours under N₂ atmosphere. (1r,4r)-4-(3-bromophenoxy)cyclohexane-1-carbaldehyde (1.7 g, 6.00 mmol, 1.0 equiv.) was added to the mixture, and stirred was continued under N₂ at 25° C. for 14 hours. The reaction mixture was quenched by aqueous NH₄Cl (20 mL) at 0° C. and extracted with EtOAc (20 mL×3). The combined organic layers were washed with brine (10 mL×1), dried over Na₂SO₄, filtered, and concentrated under reduced pressure to give a residue. The residue was purified by flash silica gel chromatography (0˜7% ethyl acetate/petroleum ether) to give 1-(((1r,4r)-4-((E)-4-(benzyloxy)but-1-en-1-yl)cyclohexyl)oxy)-3-bromobenzene (1.98 g, 4.77 mmol, 79.40% yield) as a colorless oil.

¹H NMR (400 MHz, CDCl₃-d) δ=7.40-7.33 (m, 4H), 7.32-7.27 (m, 1H), 7.16-7.09 (m, 1H), 7.07 (s, 2H), 6.83 (d, J=8.4 Hz, 1H), 5.48-5.36 (m, 1H), 5.36-5.27 (m, 1H), 4.54 (s, 2H), 4.20-4.08 (m, 1H), 3.50 (t, J=6.8 Hz, 2H), 2.41 (q, J=7.2 Hz, 2H), 2.34 (dd, J=3.6, 7.6 Hz, 1H), 2.14 (d, J=10.4 Hz, 2H), 1.76 (d, J=12.4 Hz, 2H), 1.53-1.41 (m, 2H), 1.29-1.16 (m, 2H)

### Step E. Procedure for Preparation of 1-(((1s,4r)-4-(4-(benzyloxy)butyl)cyclohexyl)oxy)-3-bromobenzene

To a solution of 1-(((1r,4r)-4-((E)-4-(benzyloxy)but-1-en-1-yl)cyclohexyl)oxy)-3-bromobenzene (1.68 g, 4.04 mmol, 1.0 equiv.) in EtOH (20 mL) was added PtO₂ (183.69 mg, 808.93 μmol, 0.2 equiv.) under N₂ atmosphere. The suspension was degassed and purged with H₂ three times. The mixture was stirred under H₂ (15 Psi) at 25° C. for 2 hours. The reaction mixture was filtered and concentrated under reduced pressure to give 1-(((1s,4r)-4-(4-(benzyloxy)butyl)cyclohexyl)oxy)-3-bromobenzene (1.32 g, crude) as a yellow oil.

### Step F. Procedure for Preparation of 4-((1r,4s)-4-(3-bromophenoxy)cyclohexyl)butan-1-ol

To a solution of 1-(((1s,4r)-4-(4-(benzyloxy)butyl)cyclohexyl)oxy)-3-bromobenzene (1.32 g, 3.16 mmol, 1.0 equiv.) in DCM (15 mL) was added TMSI (1.90 g, 9.49 mmol, 1.29 mL, 3.0 equiv.) at 0° C. under N₂. The mixture was stirred at 25° C. for 2 hours under N₂ atmosphere. The reaction mixture was quenched by the addition of H₂O (20 mL) and extracted with DCM (20 mL×2). The combined organic layers were washed with H₂O (20 mL×1), dried over Na₂SO₄, filtered, and concentrated under reduced pressure to give a residue. The residue was purified by flash silica gel chromatography (0˜20% ethyl acetate/petroleum ether) to give 4-((1r,4s)-4-(3-bromophenoxy)cyclohexyl)butan-1-ol (930 mg, 2.50 mmol, 78.98% yield) as a yellow oil.

¹H NMR (400 MHz, DMSO-d₆) δ=7.21 (t, J=8.0 Hz, 1H), 7.11 (d, J=2.0 Hz, 1H), 7.07 (d, J=8.0 Hz, 1H), 6.93 (dd, J=1.6, 8.4 Hz, 1H), 4.39-4.23 (m, 2H), 3.43-3.35 (m, 2H), 2.02 (d, J=9.6 Hz, 2H), 1.75 (d, J=12.4 Hz, 2H), 1.44-1.36 (m, 2H), 1.33-1.26 (m, 4H), 1.21-1.15 (m, 3H), 1.11-0.97 (m, 2H)

### Step G. Procedure for Preparation of 4-((1r,4s)-4-(3-bromophenoxy)cyclohexyl)butanal

A solution of DMSO (381.99 mg, 4.89 mmol, 381.99 μL, 4.0 equiv.) in DCM (4 mL) was added dropwise to a solution of oxalyl dichloride (310.29 mg, 2.44 mmol, 213.99 μL, 2.0 equiv.) in DCM (0.5 mL) at −70° C. under N₂ atmosphere. The mixture was stirred at −70° C. for 30 minutes. After which time, 4-((1r,4s)-4-(3-bromophenoxy)cyclohexyl)butan-1-ol (400.00 mg, 1.22 mmol, 1.0 equiv.) in DCM (0.5 mL) was added dropwise at −70° C. The solution was stirred for 30 minutes at −70° C. Then TEA (742.10 mg, 7.33 mmol, 1.02 mL, 6.0 equiv.) was added into the solution. The resulting reaction mixture was stirred at 25° C. for 1 hour under N₂ atmosphere. The reaction mixture was diluted with water (20 mL) and extracted with DCM (10 mL×3). The combined organic layers were washed with brine (10 mL×1), dried over Na₂SO₄, filtered, and concentrated under reduced pressure to give 4-((1r,4s)-4-(3-bromophenoxy)cyclohexyl)butanal (500 mg, crude) as a colorless oil.

¹H NMR (400 MHz, DMSO-d₆) δ=9.66 (t, J=1.6 Hz, 1H), 7.21 (t, J=8.0 Hz, 1H), 7.13-7.11 (m, 1H), 7.09-7.05 (m, 1H), 6.96-6.91 (m, 1H), 4.34-4.26 (m, 1H), 2.43-2.39 (m, 2H), 2.07-1.98 (m, 2H), 1.75 (d, J=12.4 Hz, 2H), 1.58-1.49 (m, 2H), 1.37-1.27 (m, 2H), 1.26-1.19 (m, 3H), 1.10-0.99 (m, 2H)

### Step H. Procedure for Preparation of 3-(6-(4-(4-((1r,4s)-4-(3-bromophenoxy)cyclohexyl)butyl)piperazin-1-yl)-1-methyl-1H-indazol-3-yl)piperidine-2,6-dione

To a solution of 4-((1r,4s)-4-(3-bromophenoxy)cyclohexyl)butanal (250 mg, 628.77 μmol, 1.0 equiv.) in DCM (5 mL) was added 3-(1-methyl-6-piperazin-1-yl-indazol-3-yl) piperidine-2, 6-dione (251.65 mg, 691.64 μmol, 1.1 equiv., HCl) at 25° C. The mixture was stirred at 25° C. for 16 hours. NaBH(OAc)₃ (399.78 mg, 1.89 mmol, 3 equiv.) was added to the mixture at 25° C. The reaction mixture was stirred at 25° C. for 2 hours. The reaction mixture was filtered and concentrated under reduced pressure to give a residue. The residue was purified by prep-TLC (SiO₂, DCM:MeOH=20:1) to give 3-(6-(4-(4-((1r,4s)-4-(3-bromophenoxy)cyclohexyl)butyl)piperazin-1-yl)-1-methyl-1H-indazol-3-yl)piperidine-2,6-dione (237 mg, 323.88 μmol, 51.51% yield) as a yellow solid.

MS (ESI) m/z: 636.3 [M+H]⁺.

### Step I. Procedure for preparation of tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)butyl)cyclohexyl)oxy)phenyl)picolinate

A mixture of 3-(6-(4-(4-((1r,4s)-4-(3-bromophenoxy)cyclohexyl)butyl)piperazin-1-yl)-1-methyl-1H-indazol-3-yl)piperidine-2,6-dione (237.00 mg, 372.28 μmol, 1.0 equiv.), tert-butyl 6-[8-(1, 3-benzothiazol-2-ylcarbamoyl)-3, 4-dihydro-1H-isoquinolin-2-yl]-3-(4, 4, 5, 5-tetramethyl-1, 3, 2-dioxaborolan-2-yl) pyridine-2-carboxylate (250.84 mg, 409.51 μmol, 1.1 equiv.), KF (64.88 mg, 1.12 mmol, 26.16 μL, 3.0 equiv.), and [2-(2-aminophenyl) phenyl]palladium (1⁺); bis (1-adamantyl)-butyl-phosphane; methanesulfonate (54.22 mg, 74.46 μmol, 0.2 equiv.) in dioxane (3 mL) and H₂O (0.3 mL) was degassed and purged with N₂ three times. The mixture was stirred at 100° C. for 16 hours under N₂ atmosphere. The reaction mixture was filtered and concentrated under reduced pressure to give a residue. The residue was purified by flash silica gel chromatography (0˜6% DCM/MeOH) to give tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)butyl)cyclohexyl)oxy)phenyl)picolinate (300 mg, 245.80 μmol, 66.03% yield) as a yellow solid.

MS (ESI) m/z: 1042.6 [M+H]⁺.

### Step J. Procedure for Preparation of 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)butyl)cyclohexyl)oxy)phenyl)picolinic acid

A solution of tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)butyl)cyclohexyl)oxy)phenyl)picolinate (300 mg, 287.83 μmol, 1.0 equiv.) in DCM (1.5 mL) and TFA (1.5 mL) was stirred at 25° C. for 16 hours. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)butyl)cyclohexyl)oxy)phenyl)picolinic acid (58.65 mg, 56.03 μmol, 19.47% yield) as a white solid.

MS (ESI) m/z: 986.6 [M+H]⁺.

¹H NMR (400 MHz, DMSO-d₆) δ=10.85 (s, 1H), 8.03 (d, J=8.0 Hz, 1H), 7.79 (d, J=8.0 Hz, 1H), 7.64 (dd, J=8.0, 12.0 Hz, 2H), 7.52-7.41 (m, 3H), 7.39-7.32 (m, 2H), 7.25 (t, J=8.0 Hz, 1H), 6.93 (dd, J=9.2, 16.0 Hz, 2H), 6.89-6.82 (m, 4H), 4.97 (s, 2H), 4.30-4.14 (m, 2H), 3.93-3.87 (m, 5H), 3.24 (s, 2H), 3.00 (t, J=5.6 Hz, 2H), 2.69-2.57 (m, 7H), 2.42 (s, 2H), 2.35-2.25 (m, 1H), 2.20-2.12 (m, 1H), 2.06 (d, J=9.2 Hz, 2H), 1.76 (d, J=12.4 Hz, 2H), 1.53-1.44 (m, 2H), 1.36-1.17 (m, 8H), 1.09-0.97 (m, 2H)

### Example 261. Preparation of Compound 330a

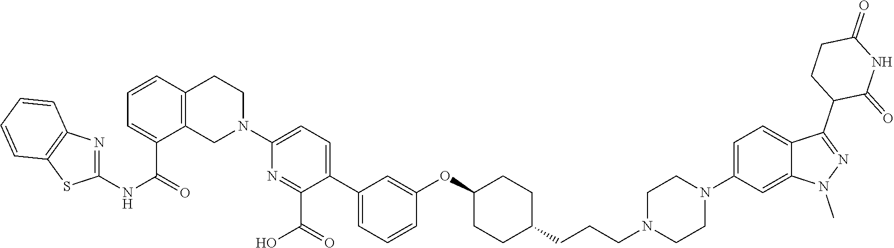 [SMILES: Cn1nc(C2CCC(=O)NC2=O)c2ccc(N3CCN(CCC[C@H]4CC[C@H](Oc5cccc(-c6ccc(N7CCc8cccc(C(=O)Nc9nc%10ccccc%10s9)c8C7)nc6C(=O)O)c5)CC4)CC3)cc21]

### 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)piperazin-1-yl)propyl)cyclohexyl)oxy)phenyl)picolinic acid

### Step A. Procedure for Preparation of ethyl (E)-3-((1r,4r)-4-(3-bromophenoxy)cyclohexyl)acrylate

A mixture of ethyl 2-diethoxyphosphorylacetate (1.19 g, 5.30 mmol, 1.05 mL, 1.0 equiv.) in THE (5 mL) was degassed and purged with N₂ three times. To this mixture was slowly added LiHMDS (1 M, 6.89 mL, 1.3 equiv.). The reaction mixture was stirred at −78° C. for 1.5 hours under a N₂ atmosphere. Then (1r,4r)-4-(3-bromophenoxy)cyclohexane-1-carbaldehyde (1.5 g, 5.30 mmol, 1.0 equiv.) in THE (15 mL) was added. The resulting mixture was slowly warm to −40° C. and stirred for 1.5 hours under N₂ atmosphere. The reaction mixture was quenched by the addition of saturated NH₄Cl (15 mL) and extracted with ethyl acetate (15 mL×2). The combined organic layers were washed with brine (15 mL×2), dried over Na₂SO₄, filtered, and concentrated under reduced pressure to give a residue. The residue was purified by flash silica gel chromatography (0˜15% ethyl acetate/petroleum ether) to give ethyl (E)-3-((1r,4r)-4-(3-bromophenoxy)cyclohexyl)acrylate (850 mg, 2.41 mmol, 45.42% yield) as a yellow oil.

¹H NMR (400 MHz, DMSO-d₆) δ=7.25-7.19 (m, 1H), 7.14 (t, J=2.0 Hz, 1H), 7.11-7.07 (m, 1H), 6.95 (dd, J=1.6, 8.4 Hz, 1H), 6.84 (dd, J=6.8, 16.0 Hz, 1H), 5.83 (dd, J=1.2, 16.0 Hz, 1H), 4.39-4.26 (m, 1H), 4.16-4.06 (m, 2H), 2.27-2.17 (m, 1H), 2.07 (d, J=10.4 Hz, 2H), 1.81 (d, J=11.6 Hz, 2H), 1.44-1.30 (m, 4H), 1.21 (t, J=7.2 Hz, 3H)

### Step B. Procedure for Preparation of ethyl 3-((1r,4r)-4-(3-bromophenoxy)cyclohexyl)propanoate

To a solution of ethyl (E)-3-((1r,4r)-4-(3-bromophenoxy)cyclohexyl)acrylate (750 mg, 2.12 mmol, 1.0 equiv.) in EtOH (5 mL) was added PtO₂ (48.21 mg, 212.31 μmol, 0.1 equiv.) under N₂. The suspension was degassed under vacuum and purged with H₂ several times. The mixture was stirred under H₂ (15 psi) at 25° C. for 2 hours. The reaction mixture was filtered and concentrated under reduced pressure to give ethyl 3-((1r,4r)-4-(3-bromophenoxy)cyclohexyl)propanoate (750 mg, crude) as a yellow oil.

¹H NMR (400 MHz, DMSO-d₆) δ=7.28-7.16 (m, 1H), 7.12 (t, J=2.0 Hz, 1H), 7.08 (d, J=8.0 Hz, 1H), 6.96-6.85 (m, 1H), 4.36-4.20 (m, 1H), 4.05 (q, J=7.2 Hz, 2H), 2.30 (t, J=7.6 Hz, 2H), 2.02 (d, J=9.6 Hz, 2H), 1.75 (d, J=12.4 Hz, 2H), 1.46 (q, J=7.2 Hz, 2H), 1.36-1.22 (m, 3H), 1.20-1.14 (m, 3H), 1.12-1.02 (m, 2H)

### Step C. Procedure for Preparation of 3-((1r,4s)-4-(3-bromophenoxy)cyclohexyl)propan-1-ol

To a solution of ethyl 3-((1r,4r)-4-(3-bromophenoxy)cyclohexyl)propanoate (750 mg, 2.11 mmol, 1.0 equiv.) in THE (8 mL) was added LiAlH₄ (80.13 mg, 2.11 mmol, 1.0 equiv.). The mixture was stirred at 0° C. for 1 hour. The reaction mixture was quenched with Na₂SO₄·10 H₂O (200 mg) and slowly warmed to 20° C. The mixture was filtered and washed with DCM (250 mL). The filtrate was concentrated under reduced pressure to give 3-((1r,4s)-4-(3-bromophenoxy)cyclohexyl)propan-1-ol (700 mg, crude) as a yellow oil.

¹H NMR (400 MHz, DMSO-d₆) δ=7.28-7.19 (m, 1H), 7.12 (d, J=2.0 Hz, 1H), 7.08 (d, J=8.0 Hz, 1H), 6.97-6.87 (m, 1H), 4.36 (t, J=5.2 Hz, 1H), 4.33-4.25 (m, 1H), 3.37 (q, J=6.4 Hz, 2H), 2.03 (d, J=9.6 Hz, 2H), 1.76 (d, J=12.0 Hz, 2H), 1.43 (d, J=7.2 Hz, 2H), 1.37-1.27 (m, 2H), 1.25-1.15 (m, 3H), 1.11-0.99 (m, 2H)

### Step D. Procedure for Preparation of 3-((1r,4r)-4-(3-bromophenoxy)cyclohexyl)propanal

A solution of oxalyl dichloride (567.30 mg, 4.47 mmol, 391.24 μL, 2.0 equiv.) in DCM (10 mL) was added dropwise to a solution of DMSO (698.45 mg, 8.94 mmol, 698.45 μL, 4.0 equiv.) in DCM (5 mL) at −78° C. under N₂ atmosphere. The mixture was stirred at −78° C. for 90 minutes. After which time 3-((1r,4s)-4-(3-bromophenoxy)cyclohexyl)propan-1-ol (700 mg, 2.23 mmol, 1.0 equiv.) in DCM (2 mL) was added dropwise at −78° C. The solution was stirred for 30 minutes at −78° C. Then TEA (1.36 g, 13.41 mmol, 1.87 mL, 6 equiv.) was added into the solution. The resulting solution was stirred at 25° C. for 1 hour under a N₂ atmosphere. The reaction mixture was quenched with H₂O (5 mL) at 25° C. and extracted with DCM (5 mL×3). The combined organic layers were dried over Na₂SO₄, filtered, and concentrated under reduced pressure to give 3-((1r,4r)-4-(3-bromophenoxy)cyclohexyl)propanal (600 mg, crude) as a yellow oil.

¹H NMR (400 MHz, DMSO-d₆) δ=9.68 (s, 1H), 7.28-7.17 (m, 1H), 7.12 (d, J=2.0 Hz, 1H), 7.08 (d, J=8.0 Hz, 1H), 6.96-6.85 (m, 1H), 2.46-2.43 (m, 2H), 2.03 (d, J=12.0 Hz, 2H), 1.75 (d, J=12.4 Hz, 2H), 1.52-1.39 (m, 2H), 1.32-1.16 (m, 4H), 1.12-0.99 (m, 2H)

### Step E. Procedure for Preparation of 3-(6-(4-(3-((1r,4s)-4-(3-bromophenoxy)cyclohexyl)propyl)piperazin-1-yl)-1-methyl-1H-indazol-3-yl)piperidine-2,6-dione

A solution of 3-(1-methyl-6-piperazin-1-yl-indazol-3-yl)piperidine-2,6-dione (210.39 mg, 642.65 mol, 1.0 equiv.) and 3-((1r,4r)-4-(3-bromophenoxy)cyclohexyl)propanal (200 mg, 642.65 μmol, 1.0 equiv.) in DCM (3 mL) was stirred at 25° C. for 0.5 hours. Then NaBH(OAc)₃ (408.61 mg, 1.93 mmol, 3 equiv.) was added. The mixture was stirred at 25° C. for 1.5 hours. The reaction mixture was concentrated under reduced pressure to give a residue. The residue was purified by prep-TLC (SiO₂, DCM:MeOH=10:1) to give 3-(6-(4-(3-((1r,4s)-4-(3-bromophenoxy)cyclohexyl)propyl)piperazin-1-yl)-1-methyl-1H-indazol-3-yl)piperidine-2,6-dione (190 mg, 248.60 μmol, 38.68% yield) as a yellow oil.

MS (ESI) m/z: 622.4 [M+H]⁺.

### Step F. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)piperazin-1-yl)propyl)cyclohexyl)oxy)phenyl)picolinate

To a solution of 3-(6-(4-(3-((1r,4s)-4-(3-bromophenoxy)cyclohexyl)propyl)piperazin-1-yl)-1-methyl-1H-indazol-3-yl)piperidine-2,6-dione (190 mg, 305.17 μmol, 1.0 equiv.) and tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (224.32 mg, 366.21 μmol, 1.2 equiv.) in dioxane (3 mL) and H₂O (0.3 mL) was added [2-(2-aminophenyl)phenyl]palladium(1⁺); bis(1-adamantyl)-butyl-phosphane; methanesulfonate (22.22 mg, 30.52 μmol, 0.1 equiv.) and KF (53.19 mg, 915.52 μmol, 21.45 μL, 3.0 equiv.). The mixture was stirred at 100° C. for 16 hours. The reaction mixture was concentrated under reduced pressure to give a residue. The residue was purified by flash silica gel chromatography (0˜6% dichloromethane/methanol) to give tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)piperazin-1-yl)propyl)cyclohexyl)oxy)phenyl)picolinate (210 mg, 180.72 μmol, 59.22% yield) as a yellow oil.

MS (ESI) m/z: 515.1 [M/2+H]⁺.

### Step G. Procedure for Preparation of 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)piperazin-1-yl)propyl)cyclohexyl)oxy)phenyl)picolinic acid

To a solution of tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)piperazin-1-yl)propyl)cyclohexyl)oxy)phenyl)picolinate (210 mg, 204.23 μmol, 1.0 equiv.) in DCM (1 mL) was added TFA (2 mL). The mixture was stirred at 25° C. for 16 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)piperazin-1-yl)propyl)cyclohexyl)oxy)phenyl)picolinic acid (86.61 mg, 85.10 μmol, 41.67% yield) as a yellow solid.

MS (ESI) m/z: 972.6 [M+H]⁺.

¹H NMR (400 MHz, DMSO-d₆) δ=13.36-12.34 (m, 1H), 10.85 (s, 1H), 8.03 (d, J=7.6 Hz, 1H), 7.79 (d, J=8.0 Hz, 1H), 7.68-7.60 (m, 2H), 7.54-7.45 (m, 2H), 7.43 (d, J=8.4 Hz, 1H), 7.35 (q, J=7.2 Hz, 2H), 7.25 (t, J=7.6 Hz, 1H), 6.93 (dd, J=9.2, 14.0 Hz, 2H), 6.89-6.82 (m, 4H), 4.97 (s, 2H), 4.29-4.18 (m, 2H), 3.90-3.88 (m, 5H), 3.26-2.24 (m, 2H), 3.02-3.01 (m, 2H), 2.69-2.61 (m, 5H), 2.55-2.53 (m, 2H), 2.45-2.43 (m, 2H), 2.34-2.25 (m, 1H), 2.17-2.15 (m, 1H), 2.07-2.05 (m, 2H), 1.79-1.77 (m, 2H), 1.53-1.51 (m, 2H), 1.39-1.13 (m, 6H), 1.11-0.98 (m, 2H)

### Example 262. Preparation of Compound 213

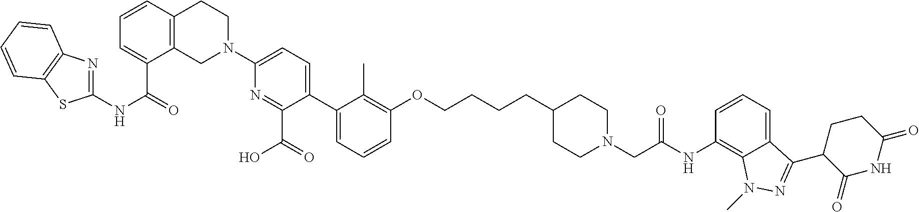 [SMILES: Cc1c(OCCCCC2CCN(CC(=O)Nc3cccc4c(C5CCC(=O)NC5=O)nn(C)c34)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(4-(1-(2-((3-(2,6-dioxopiperidin-3-yl)-1-methyl-1H-indazol-7-yl)amino)-2-oxoethyl)piperidin-4-yl)butoxy)-2-methylphenyl)picolinic acid

### Step A. Procedure for Preparation of methyl 2-(4-(4-(2-methyl-3-(4,4,5,5-tetramethyl-1,3,2-dioxaborolan-2-yl)phenoxy)butyl)piperidin-1-yl)acetate

A solution of tert-butyl 4-(3-(4-bromo-3-methylphenoxy)propyl)piperidine-1-carboxylate (2.2 g, 1 equiv., 5.3 mmol), 4,4,4′,4′,5,5,5′,5′-octamethyl-2,2′-bi(1,3,2-dioxaborolane) (2.0 g, 1.5 equiv, 8.0 mmol), potassium acetate (1.6 g, 3 equiv., 16 mmol), and PdCl2(dppf)-CH₂Cl₂ adduct (0.22 g, 0.05 equiv., 0.26 mmol) in 1,4-dioxane (15 mL) was purged with nitrogen gas and heated to 85° C. After 16 h the reaction solution was concentrated, and the resulting crude material was purified by silica gel column chromatography (0-100% ethyl acetate in heptane) to provide methyl 2-(4-(4-(2-methyl-3-(4,4,5,5-tetramethyl-1,3,2-dioxaborolan-2-yl)phenoxy)butyl)piperidin-1-yl)acetate (2.6 g, 5.7 mmol) as a colorless oil.

### Step B. Procedure for Preparation of tert-Butyl 6-(8-(benzo[d]thiazol-2-ylcarbamoyl)-3,4-dihydroisoquinolin-2(1H)-yl)-3-(3-(4-(1-(2-methoxy-2-oxoethyl)piperidin-4-yl)butoxy)-2-methylphenyl)picolinate

A solution of tert-butyl 6-(8-(benzo[d]thiazol-2-ylcarbamoyl)-3,4-dihydroisoquinolin-2(1H)-yl)-3-bromopicolinate (500 mg, 1 equiv., 884 μmol), methyl 2-(4-(4-(2-methyl-3-(4,4,5,5-tetramethyl-1,3,2-dioxaborolan-2-yl)phenoxy)butyl)piperidin-1-yl)acetate (512 mg, 1.3 equiv., 1.15 mmol), and CataCXium PD G3 (64.4 mg, 0.1 equiv., 88.4 μmol) in 1,4-dioxane (7.5 mL) and K₃PO₄ (1.5 M, 2.5 mL) was heated in the microwave at 100° C. After 40 minutes, the reaction vessel was removed from microwave and heated to 80° C. by conventional heating. After 12 hours, the reaction solution was filtered and concentrated to provide crude tert-butyl 6-(8-(benzo[d]thiazol-2-ylcarbamoyl)-3,4-dihydroisoquinolin-2(1H)-yl)-3-(3-(4-(1-(2-methoxy-2-oxoethyl)piperidin-4-yl)butoxy)-2-methylphenyl)picolinate, which was carried forward without further purification.

### Step C. Procedure for Preparation of 2-(4-(4-(3-(6-(8-(Benzo[d]thiazol-2-ylcarbamoyl)-3,4-dihydroisoquinolin-2(1H)-yl)-2-(tert-butoxycarbonyl)pyridin-3-yl)-2-methylphenoxy)butyl)piperidin-1-yl)acetic acid

To a solution of tert-butyl 6-(8-(benzo[d]thiazol-2-ylcarbamoyl)-3,4-dihydroisoquinolin-2(1H)-yl)-3-(3-(4-(1-(2-methoxy-2-oxoethyl)piperidin-4-yl)butoxy)-2-methylphenyl)picolinate (711 mg, 884 μmol, 1 equiv.) in methanol (5 mL) was added an aqueous solution ofNaOH (1 M, 1 mL). The reaction solution was stirred at 60° C. After 3 hours, the reaction solution was concentrated and purified by silica gel column chromatography to provide 2-(4-(4-(3-(6-(8-(Benzo[d]thiazol-2-ylcarbamoyl)-3,4-dihydroisoquinolin-2(1H)-yl)-2-(tert-butoxycarbonyl)pyridine-3-yl)-2-methylphenoxy)butyl)piperidin-1-yl)acetic acid (278 mg, 352 μmol, 40% yield over two steps).

MS (ESI) m/z: 791.2 [M+H]⁺.

### Step D. Procedure for Preparation of tert-butyl 6-(8-(benzo[d]thiazol-2-ylcarbamoyl)-3,4-dihydroisoquinolin-2(1H)-yl)-3-(3-(4-(1-(2-((3-(2,6-dioxopiperidin-3-yl)-1-methyl-1H-indazol-7-yl)amino)-2-oxoethyl)piperidin-4-yl)butoxy)-2-methylphenyl)picolinate

A solution of 2-(4-(4-(3-(6-(8-(benzo[d]thiazol-2-ylcarbamoyl)-3,4-dihydroisoquinolin-2(1H)-yl)-2-(tert-butoxycarbonyl)pyridine-3-yl)-2-methylphenoxy)butyl)piperidin-1-yl)acetic acid (60 mg, 1 equiv., 76 μmol), 3-(7-amino-1-methyl-1H-indazol-3-yl)piperidine-2,6-dione (24 mg, 1.2 equiv., 91 μmol), N,N,N′,N′-Tetramethylchloroformamidinium-hexafluorophosphate (26 mg, 1.2 equiv., 91 μmol), and 1-methylimidazole (22 mg, 21 μL, 3.5 equiv., 0.27 mmol) in MeCN (5 mL) was stirred at room temperature. After 16 h the reaction solution was filtered and concentrated to provide crude tert-butyl 6-(8-(benzo[d]thiazol-2-ylcarbamoyl)-3,4-dihydroisoquinolin-2(1H)-yl)-3-(3-(4-(1-(2-((3-(2,6-dioxopiperidin-3-yl)-1-methyl-1H-indazol-7-yl)amino)-2-oxoethyl)piperidin-4-yl)butoxy)-2-methylphenyl)picolinate, which was carried forward without further purification.

MS (ESI) m/z: 516.0 [M+H/2]⁺.

### Step E. Procedure for Preparation of 6-(8-(benzo[d]thiazol-2-ylcarbamoyl)-3,4-dihydroisoquinolin-2(1H)-yl)-3-(3-(4-(1-(2-((3-(2,6-dioxopiperidin-3-yl)-1-methyl-1H-indazol-7-yl)amino)-2-oxoethyl)piperidin-4-yl)butoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(4-(1-(2-((3-(2,6-dioxopiperidin-3-yl)-1-methyl-1H-indazol-7-yl)amino)-2-oxoethyl)piperidin-4-yl)butoxy)-2-methylphenyl)picolinate (78 mg, 76 μmol, 1 equiv.) in DCM (4 mL) was added TFA (1 mL). The reaction solution was stirred at room temperature. After 16 hours, the reaction solution was concentrated. The resulting residue was re-suspended in DMSO and purified by reverse-phase HPLC to provide 6-(8-(benzo[d]thiazol-2-ylcarbamoyl)-3,4-dihydroisoquinolin-2(1H)-yl)-3-(3-(4-(1-(2-((3-(2,6-dioxopiperidin-3-yl)-1-methyl-1H-indazol-7-yl)amino)-2-oxoethyl)piperidin-4-yl)butoxy)-2-methylphenyl)picolinic acid (17 mg, 17 μmol, 23% yield) as a white solid.

MS (ESI) m/z: 974 [M+H]⁺. ¹H NMR is consistent with structure.

### Example 263. Preparation of Compound 331a

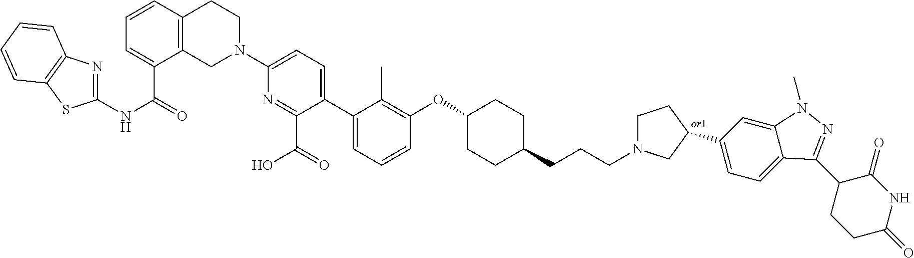 [SMILES: Cc1c(O[C@H]2CC[C@H](CCCN3CC[C@H](c4ccc5c(C6CCC(=O)NC6=O)nn(C)c5c4)C3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(3-((3R)-3-(3-(2,6-dioxopiperidin-3-yl)-1-methyl-1H-indazol-6-yl)pyrrolidin-1-yl)propyl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1S,4r)-4-(3-((3R*)-3-(3-(2,6-dioxopiperidin-3-yl)-1-methyl-1H-indazol-6-yl)pyrrolidin-1-yl)propyl)cyclohexyl)oxy)-2-methylphenyl)picolinic acid]

### Step A. Procedure for Preparation of tert-butyl (3R)-3-[3-(2,6-dioxo-3-piperidyl)-1-methyl-indazol-6-yl]pyrrolidine-1-carboxylate

To a solution of tert-butyl (3R)-3-[3-(2,6-dibenzyloxy-3-pyridyl)-1-methyl-indazol-6-yl]pyrrolidine-1-carboxylate (4.2 g, 7.11 mmol, 1 equiv.) in EtOH (20 mL) and THE (20 mL) was added Pd/C (2.10 g, 1.97 mmol, 10% purity, 0.278 equiv.), Pd(OH)₂ (2.10 g, 14.95 mmol, 2.10 equiv.) and AcOH (426.96 mg, 7.11 mmol, 407.02 μL, 1 equiv.) under N₂ atmosphere. The suspension was degassed and purged with H₂ three times. The mixture was stirred under H₂ (15 Psi) at 25° C. for 16 hours. The reaction mixture was filtered, and the filter cake was washed with THE (200 mL). The filtrate was concentrated to afford tert-butyl (3R)-3-[3-(2,6-dioxo-3-piperidyl)-1-methyl-indazol-6-yl]pyrrolidine-1-carboxylate (3.05 g, crude) as a brown solid, which was used in the next step without further purification.

### Step B. Procedure for Preparation of 3-[1-methyl-6-[(3R)-pyrrolidin-3-yl]indazol-3-yl]piperidine-2,6-dione

To a solution of tert-butyl (3R)-3-[3-(2,6-dioxo-3-piperidyl)-1-methyl-indazol-6-yl]pyrrolidine-1-carboxylate (3.0 g, 7.27 mmol, 1 equiv.) in dioxane (10 mL) was added HCl/dioxane (4 M, 21.05 mL, 11.58 equiv.). The mixture was stirred at 25° C. for 16 hours. The reaction mixture was filtered to afford 3-[1-methyl-6-[(3R)-pyrrolidin-3-yl]indazol-3-yl]piperidine-2,6-dione (1345.01 mg, 7.26 mmol, 99.80% yield, HCl) as a yellow solid. MS (ESI) m/z: 313.2 [M+H]⁺. ¹H NMR (400 MHz, DMSO-d₆) δ=10.89 (s, 1H), 9.88-9.57 (m, 2H), 7.68 (d, J=8.4 Hz, 1H), 7.63 (s, 1H), 7.13 (dd, J=1.2, 8.4 Hz, 1H), 4.36 (dd, J=5.2, 9.6 Hz, 1H), 4.00 (s, 3H), 3.67-3.58 (m, 2H), 3.44-3.37 (m, 1H), 3.29-3.15 (m, 2H), 2.73-2.55 (m, 2H), 2.43-2.32 (m, 2H), 2.17 (dd, J=5.2, 13.6 Hz, 1H), 2.08-1.98 (m, 1H).

### Step C. Procedure for Preparation of 3-((1r,4r)-4-(3-bromo-2-methylphenoxy)cyclohexyl)propanal

A mixture of 3-((1r,4s)-4-(3-bromo-2-methylphenoxy)cyclohexyl)propan-1-ol (500 mg, 1.53 mmol, 1.0 equiv.) and DMP (972.04 mg, 2.29 mmol, 710.04 μL, 1.5 equiv.) in DCM (10 mL) was degassed and purged with N₂ three times. The mixture was stirred at 35° C. for 3 hours under N₂ atmosphere. The mixture was filtered and washed with DCM (20 mL). The filtrate was concentrated under reduced pressure to give a residue. The residue was purified by flash silica gel chromatography (0-10% ethyl acetate/petroleum ether) to give 3-((1r,4r)-4-(3-bromo-2-methylphenoxy)cyclohexyl)propanal (530 mg, 1.63 mmol, 53.33% yield) as a white solid.

¹H NMR (400 MHz, DMSO-d₆) δ=9.68 (s, 1H), 7.17-7.11 (m, 1H), 7.10-6.98 (m, 2H), 4.22 (m, 1H), 2.45 (m, 2H), 2.20 (s, 3H), 2.04 (m, 2H), 1.75 (m, 2H), 1.45 (q, J=7.2 Hz, 2H), 1.36-1.22 (m, 3H), 1.04 (q, J=12.0 Hz, 2H).

### Step D. Procedure for Preparation of 3-(6-((R)-1-(3-((1r,4s)-4-(3-bromo-2-methylphenoxy)cyclohexyl)propyl)pyrrolidin-3-yl)-1-methyl-1H-indazol-3-yl)piperidine-2,6-dione

To a solution of 3-((1r,4r)-4-(3-bromo-2-methylphenoxy)cyclohexyl)propanal (100 mg, 307.47 μmol, 1.0 equiv.), 3-(1-methyl-6-((R)-pyrrolidin-3-yl)-1H-indazol-3-yl)piperidine-2,6-dione (115.25 mg, 368.96 μmol, 1.2 equiv.), and NaOAc (25.22 mg, 307.47 μmol, 1.0 equiv.) in DCM (2 mL) was added NaBH(OAc)₃ (325.82 mg, 1.54 mmol, 5.0 equiv.). The resulting mixture was stirred at 25° C. for 1 hour. After completion, the reaction mixture was poured into water (10 mL), and then extracted with ethyl acetate (10 mL×3). The combined organic layers were washed with brine (10 mL), dried over anhydrous sodium sulfate, filtered, and concentrated under reduced pressure to give 3-(6-((R)-1-(3-((1r,4s)-4-(3-bromo-2-methylphenoxy)cyclohexyl)propyl)pyrrolidin-3-yl)-1-methyl-1H-indazol-3-yl)piperidine-2,6-dione (200 mg, crude) as a yellow oil.

MS (ESI) m/z: 621.4 [M+H]⁺.

### Step E. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(3-((3R)-3-(3-(2,6-dioxopiperidin-3-yl)-1-methyl-1H-indazol-6-yl)pyrrolidin-1-yl)propyl)cyclohexyl)oxy)-2-methylphenyl)picolinate

A mixture of 3-(6-((R)-1-(3-((1r,4s)-4-(3-bromo-2-methylphenoxy)cyclohexyl)propyl)pyrrolidin-3-yl)-1-methyl-1H-indazol-3-yl)piperidine-2,6-dione (200 mg, 248.71 μmol, 1.0 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (182.82 mg, 298.45 μmol, 1.2 equiv.), Ad₂nBuP Pd G₃ (cataCXium® A Pd G₃) (36.23 mg, 49.74 μmol, 0.2 equiv.), and KF (72.25 mg, 1.24 mmol, 29.13 μL, 5.0 equiv.) in dioxane (6 mL) and H₂O (0.6 mL) was degassed and purged with N₂ three times. The mixture was stirred at 100° C. for 3 hours under N₂ atmosphere. The reaction mixture was concentrated under reduced pressure to give a residue. The residue was purified by prep-TLC (SiO₂, petroleum ether: ethyl acetate=0:1). The product thus obtained was purified by prep-TLC (SiO₂, DCM:MeOH=10:1) to give tert-butyl 6-(8-(benzo[d]thiazol-2-ylcarbamoyl)-3,4-dihydroisoquinolin-2(1H)-yl)-3-(3-(((1s,4r)-4-(3-((3R)-3-(3-(2,6-dioxopiperidin-3-yl)-1-methyl-1H-indazol-6-yl)pyrrolidin-1-yl)propyl)cyclohexyl)oxy)-2-methylphenyl)picolinate (120 mg, 93.24 μmol, 37.49% yield) as a yellow oil.

MS (ESI) m/z: 1027.6 [M+H]⁺.

### Step F. Procedure for Preparation of 6-(8-(benzo[d]thiazol-2-ylcarbamoyl)-3,4-dihydroisoquinolin-2(1H)-yl)-3-(3-(((1s,4r)-4-(3-((3R)-3-(3-(2,6-dioxopiperidin-3-yl)-1-methyl-1H-indazol-6-yl)pyrrolidin-1-yl)propyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1S,4r)-4-(3-((3R*)-3-(3-(2,6-dioxopiperidin-3-yl)-1-methyl-1H-indazol-6-yl)pyrrolidin-1-yl)propyl)cyclohexyl)oxy)-2-methylphenyl)picolinic acid]

A solution of tert-butyl 6-(8-(benzo[d]thiazol-2-ylcarbamoyl)-3,4-dihydroisoquinolin-2(1H)-yl)-3-(3-(((1 s,4r)-4-(3-((3R)-3-(3-(2,6-dioxopiperidin-3-yl)-1-methyl-1H-indazol-6-yl)pyrrolidin-1-yl)propyl)cyclohexyl)oxy)-2-methylphenyl)picolinate (120 mg, 116.81 μmol, 1.0 equiv.) in TFA (1.5 mL) and DCM (0.5 mL) was stirred at 35° C. for 1.5 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-(3-((3R)-3-(3-(2,6-dioxopiperidin-3-yl)-1-methyl-1H-indazol-6-yl)pyrrolidin-1-yl)propyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1S,4r)-4-(3-((3R*)-3-(3-(2,6-dioxopiperidin-3-yl)-1-methyl-1H-indazol-6-yl)pyrrolidin-1-yl)propyl)cyclohexyl)oxy)-2-methylphenyl)picolinic acid](65.65 mg, 65.30 μmol, 55.90% yield) as a yellow solid.

MS (ESI) m/z: 971.7 [M+H]⁺.

¹H NMR (400 MHz, DMSO-d₆) δ=12.96-12.77 (m, 1H), 12.67-12.45 (m, 1H), 10.93 (d, J=1.2 Hz, 1H), 8.08-7.98 (m, 1H), 7.79 (m, 1H), 7.71 (d, J=8.8 Hz, 1H), 7.64-7.54 (m, 2H), 7.49-7.42 (m, 3H), 7.40-7.32 (m, 2H), 7.17-7.05 (m, 2H), 6.95 (m, 2H), 6.62 (d, J=7.6 Hz, 1H), 5.05-4.91 (m, 2H), 4.39-4.31 (m, 1H), 4.26-4.16 (m, 1H), 4.00 (s, 3H), 3.92 (m, 2H), 3.62-3.54 (m, 1H), 3.27-3.17 (m, 4H), 3.03 (m, 2H), 2.61 (m, 4H), 2.23-2.15 (m, 2H), 2.13-2.07 (m, 2H), 1.87 (s, 3H), 1.85-1.79 (m, 2H), 1.74-1.65 (m, 2H), 1.42-1.23 (m, 6H), 1.17-1.01 (m, 3H).

### Example 264. Preparation of Compound 331b

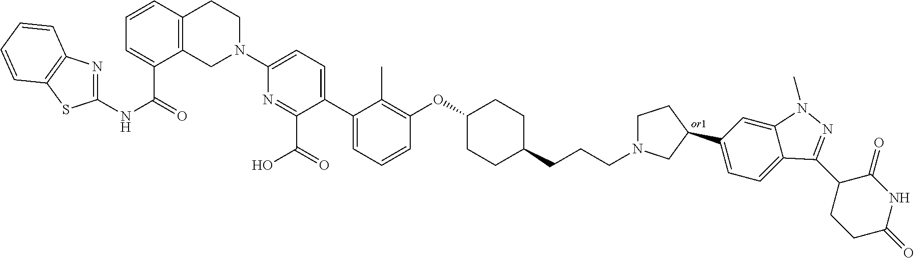 [SMILES: Cc1c(O[C@H]2CC[C@H](CCCN3CC[C@@H](c4ccc5c(C6CCC(=O)NC6=O)nn(C)c5c4)C3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-(3-((3S)-3-(3-(2,6-dioxopiperidin-3-yl)-1-methyl-1H-indazol-6-yl)pyrrolidin-1-yl)propyl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1R,4r)-4-(3-((3S*)-3-(3-(2,6-dioxopiperidin-3-yl)-1-methyl-1H-indazol-6-yl)pyrrolidin-1-yl)propyl)cyclohexyl)oxy)-2-methylphenyl)picolinic acid]

### Step A. Procedure for Preparation of tert-butyl 3-[3-(2,6-dibenzyloxy-3-pyridyl)-1-methyl-indazol-6-yl]pyrrolidine-1-carboxylate

A solution of 6-bromo-3-(2,6-dibenzyloxy-3-pyridyl)-1-methyl-indazole (15 g, 29.98 mmol, 1 equiv.), tert-butyl 3-bromopyrrolidine-1-carboxylate (9.75 g, 38.97 mmol, 1.3 equiv.), Ir[dF(CF₃)ppy]2(dtbpy)(PF₆) (336.31 mg, 299.77 μmol, 0.01 equiv.), NiCl₂.dtbbpy (178.96 mg, 449.65 μmol, 0.015 equiv.), TTMSS (7.45 g, 29.98 mmol, 9.25 mL, 1 equiv.), and Na₂CO₃ (6.35 g, 59.95 mmol, 2 equiv.) in DME (2 mL) was sealed and placed under nitrogen. The reaction was stirred and irradiated with a 10 W blue LED lamp (3 cm away) with cooling water to keep the reaction temperature at 25° C. for 14 hours. The reaction mixture was filtered, diluted with H₂O (500 mL), and extracted with ethyl acetate (200 mL×3). The combined organic layers were washed with brine (300 mL), dried over Na₂SO₄, filtered, and concentrated under reduced pressure to give a residue. The residue was purified by flash silica gel chromatography (0-35% ethyl acetate/petroleum ether) to afford tert-butyl 3-[3-(2,6-dibenzyloxy-3-pyridyl)-1-methyl-indazol-6-yl]pyrrolidine-1-carboxylate (10.5 g, 17.06 mmol, 56.92% yield) as a yellow oil. MS (ESI) m/z: 591.3 [M+H]⁺.

### Step B. Procedure for Preparation of tert-butyl (3R)-3-[3-(2,6-dibenzyloxy-3-pyridyl)-1-methyl-indazol-6-yl]pyrrolidine-1-carboxylate and tert-butyl (3S)-3-[3-(2,6-dibenzyloxy-3-pyridyl)-1-methyl-indazol-6-yl]pyrrolidine-1-carboxylate

The stereoisomers of tert-butyl 3-[3-(2,6-dibenzyloxy-3-pyridyl)-1-methyl-indazol-6-yl]pyrrolidine-1-carboxylate were separated by SFC to afford tert-butyl (3S)-3-[3-(2,6-dibenzyloxy-3-pyridyl)-1-methyl-indazol-6-yl]pyrrolidine-1-carboxylate (3.5 g, 5.75 mmol, 32.33% yield) was obtained as a yellow oil and tert-butyl (3R)-3-[3-(2,6-dibenzyloxy-3-pyridyl)-1-methyl-indazol-6-yl]pyrrolidine-1-carboxylate (4.2 g, 6.97 mmol, 39.20% yield) as a yellow oil.

MS (ESI) m/z: 591.3 [M+H]⁺. MS (ESI) m/z: 591.3 [M+H]⁺.

### Step C. Procedure for Preparation of tert-butyl (3S)-3-[3-(2,6-dioxo-3-piperidyl)-1-methyl-indazol-6-yl]pyrrolidine-1-carboxylate

To a solution of tert-butyl (3S)-3-[3-(2,6-dibenzyloxy-3-pyridyl)-1-methyl-indazol-6-yl]pyrrolidine-1-carboxylate (3.5 g, 5.93 mmol, 1 equiv.) in EtOH (20 mL) and THE (20 mL) was added Pd/C (2 g, 1.88 mmol, 10% purity, 0.317 equiv.), Pd(OH)₂ (2 g, 14.24 mmol, 2.40 equiv.), and AcOH (355.81 mg, 5.93 mmol, 339.19 μL, 1 equiv.) under N₂ atmosphere. The suspension was degassed and purged with H₂ three times. The mixture was stirred under H₂ (15 Psi) at 25° C. for 16 hours. The reaction mixture was filtered, and the filter cake was washed with THE (200 mL). The filtrate was concentrated under vacuum to afford tert-butyl (3S)-3-[3-(2,6-dioxo-3-piperidyl)-1-methyl-indazol-6-yl]pyrrolidine-1-carboxylate (2.7 g, crude) as a brown solid, which was used in the next step without further purification.

### Step D. Procedure for Preparation of 3-[1-methyl-6-[(3S)-pyrrolidin-3-yl]indazol-3-yl]piperidine-2,6-dione

To a solution of tert-butyl (3S)-3-[3-(2,6-dioxo-3-piperidyl)-1-methyl-indazol-6-yl]pyrrolidine-1-carboxylate (2.65 g, 6.42 mmol, 1 eq) in dioxane (10 mL) was added HCl/dioxane (4 M, 20.78 mL, 12.94 equiv.). The mixture was stirred at 25° C. for 16 hours. The reaction mixture was filtered to afford 3-[1-methyl-6-[(3S)-pyrrolidin-3-yl]indazol-3-yl]piperidine-2,6-dione (1287.35 mg, 6.30 mmol, 98.13% yield, 2HCl) as a pink solid. MS (ESI) m/z: 313.3 [M+H]⁺. ¹H NMR (400 MHz, DMSO-d₆) δ=10.89 (s, 1H), 9.84-9.64 (m, 2H), 7.68 (d, J=8.4 Hz, 1H), 7.62 (s, 1H), 7.12 (d, J=8.4 Hz, 1H), 4.36 (dd, J=5.2, 9.6 Hz, 1H), 3.99 (s, 3H), 3.67-3.58 (m, 2H), 3.44-3.37 (m, 1H), 3.28-3.14 (m, 2H), 2.72-2.55 (m, 2H), 2.43-2.31 (m, 2H), 2.20-2.12 (m, 1H), 2.07-1.97 (m, 1H).

### Step E. Procedure for Preparation of 3-(6-((S)-1-(3-((1r,4r)-4-(3-bromo-2-methylphenoxy)cyclohexyl)propyl)pyrrolidin-3-yl)-1-methyl-1H-indazol-3-yl)piperidine-2,6-dione

To a solution of 3-((1r,4r)-4-(3-bromo-2-methylphenoxy)cyclohexyl)propanal (100 mg, 307.47 mol, 1.0 equiv.) and 3-[1-methyl-6-[(3S)-pyrrolidin-3-yl]indazol-3-yl]piperidine-2,6-dione (105.65 mg, 338.21 μmol, 1.1 equiv.) in DCM (5 mL) was added NaOAc (50.45 mg, 614.93 μmol, 2.0 equiv.) at 25° C. for 1 hour. Then sodium triacetoxyborohydride (325.82 mg, 1.54 mmol, 5.0 equiv.) was added to the mixture which was stirred at 25° C. for 1 hour. The reaction mixture was partitioned between DCM (15 mL) and water (8 mL). The organic phase was separated, dried over Na₂SO₄, filtered, and concentrated under reduced pressure to give 3-(6-((S)-1-(3-((1r,4r)-4-(3-bromo-2-methylphenoxy)cyclohexyl)propyl)pyrrolidin-3-yl)-1-methyl-1H-indazol-3-yl)piperidine-2,6-dione (130 mg, crude) as a yellow oil.

MS (ESI) m/z: 621.2 [M+H]⁺.

### Step F. Procedure for Preparation of tert-butyl 6-(8-(benzo[d]thiazol-2-ylcarbamoyl)-3,4-dihydroisoquinolin-2(1H)-yl)-3-(3-(((1r,4r)-4-(3-((3S)-3-(3-(2,6-dioxopiperidin-3-yl)-1-methyl-1H-indazol-6-yl)pyrrolidin-1-yl)propyl)cyclohexyl)oxy)-2-methylphenyl)picolinate

A mixture of 3-(6-((S)-1-(3-((1r,4r)-4-(3-bromo-2-methylphenoxy)cyclohexyl)propyl)pyrrolidin-3-yl)-1-methyl-1H-indazol-3-yl)piperidine-2,6-dione (130 mg, 209.14 μmol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (140.92 mg, 230.05 μmol, 1.1 equiv.), Ad₂nBuP Pd G₃ (30.46 mg, 41.83 μmol, 0.2 equiv.), and KF (60.75 mg, 1.05 mmol, 24.50 μL, 5.0 equiv.) in dioxane (6 mL) and H₂O (0.6 mL) was degassed and purged with N₂ three times. The mixture was stirred at 80° C. for 12 hours under N₂ atmosphere. The reaction mixture was concentrated under reduced pressure to give a residue. The residue was purified by prep-TLC (SiO₂, DCM:MeOH=12:1) to give tert-butyl 6-(8-(benzo[d]thiazol-2-ylcarbamoyl)-3,4-dihydroisoquinolin-2(1H)-yl)-3-(3-(((1r,4r)-4-(3-((3S)-3-(3-(2,6-dioxopiperidin-3-yl)-1-methyl-1H-indazol-6-yl)pyrrolidin-1-yl)propyl)cyclohexyl)oxy)-2-methylphenyl)picolinate (100 mg, 70.09 μmol, 33.51% yield) as a yellow solid.

MS (ESI) m/z: 514.5 [M/2+H]⁺.

### Step G. Procedure for Preparation of 6-(8-(benzo[d]thiazol-2-ylcarbamoyl)-3,4-dihydroisoquinolin-2(1H)-yl)-3-(3-(((1r,4r)-4-(3-((3S)-3-(3-(2,6-dioxopiperidin-3-yl)-1-methyl-1H-indazol-6-yl)pyrrolidin-1-yl)propyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1R,4r)-4-(3-((3S*)-3-(3-(2,6-dioxopiperidin-3-yl)-1-methyl-1H-indazol-6-yl)pyrrolidin-1-yl)propyl)cyclohexyl)oxy)-2-methylphenyl)picolinic acid]

A solution of tert-butyl 6-(8-(benzo[d]thiazol-2-ylcarbamoyl)-3,4-dihydroisoquinolin-2(1H)-yl)-3-(3-(((1R,4r)-4-(3-((3S)-3-(3-(2,6-dioxopiperidin-3-yl)-1-methyl-1H-indazol-6-yl)pyrrolidin-1-yl)propyl)cyclohexyl)oxy)-2-methylphenyl)picolinate (100 mg, 97.34 μmol, 1.0 equiv.) in TFA (3 mL) and DCM (1 mL) was stirred at 40° C. for 2 hours. The reaction mixture was concentrated under reduced pressure to give a residue. The residue was purified by reverse-phase HPLC to give 6-(8-(benzo[d]thiazol-2-ylcarbamoyl)-3,4-dihydroisoquinolin-2(1H)-yl)-3-(3-(((1r,4r)-4-(3-((3S)-3-(3-(2,6-dioxopiperidin-3-yl)-1-methyl-1H-indazol-6-yl)pyrrolidin-1-yl)propyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1R,4r)-4-(3-((3S*)-3-(3-(2,6-dioxopiperidin-3-yl)-1-methyl-1H-indazol-6-yl)pyrrolidin-1-yl)propyl)cyclohexyl)oxy)-2-methylphenyl)picolinic acid](37.80 mg, 37.28 μmol, 38.30% yield) as a white solid.

MS (ESI) m/z: 971.7 [M+H]⁺.

¹H NMR (400 MHz, DMSO-d₆) δ=10.87 (s, 1H), 8.15 (s, 1H), 8.02 (d, J=8.0 Hz, 1H), 7.78 (d, J=8.0 Hz, 1H), 7.66-7.60 (m, 2H), 7.48-7.41 (m, 4H), 7.37 (dd, J=8.0, 10.0 Hz, 2H), 7.09 (s, 2H), 6.93 (dd, J=8.8, 13.2 Hz, 2H), 6.62 (d, J=8.0 Hz, 1H), 4.97 (s, 2H), 4.38-4.27 (m, 1H), 4.25-4.12 (m, 1H), 3.96 (s, 3H), 3.94-3.87 (m, 2H), 3.52-3.45 (m, 1H), 3.16-3.07 (m, 1H), 3.02 (t, J=4.8 Hz, 2H), 2.93-2.80 (m, 2H), 2.61 (s, 3H), 2.36-2.27 (m, 4H), 2.22-2.12 (m, 2H), 2.07 (dd, J=2.0, 8.4 Hz, 2H), 1.87 (s, 3H), 1.82-1.76 (m, 2H), 1.57-1.48 (m, 2H), 1.40-1.32 (m, 2H), 1.30-1.22 (m, 3H), 1.12-1.00 (m, 2H)

### Example 265. Preparation of Compound 332a

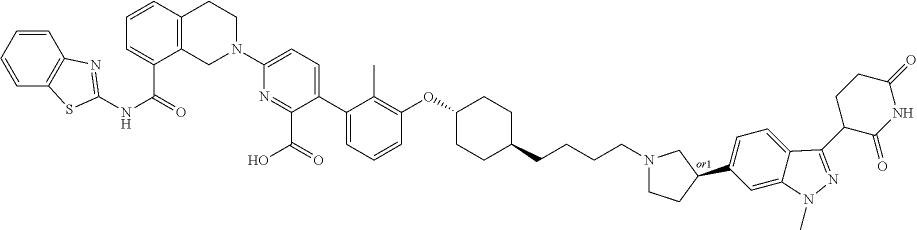 [SMILES: Cc1c(O[C@H]2CC[C@H](CCCCN3CC[C@H](c4ccc5c(C6CCC(=O)NC6=O)nn(C)c5c4)C3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(4-((3R)-3-(3-(2,6-dioxopiperidin-3-yl)-1-methyl-1H-indazol-6-yl)pyrrolidin-1-yl)butyl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1S,4r)-4-(4-((3R*)-3-(3-(2,6-dioxopiperidin-3-yl)-1-methyl-1H-indazol-6-yl)pyrrolidin-1-yl)butyl)cyclohexyl)oxy)-2-methylphenyl)picolinic acid]

### Step A. Procedure for Preparation of 3-(6-((R)-1-(4-((1r,4s)-4-(3-bromo-2-methylphenoxy)cyclohexyl)butyl)pyrrolidin-3-yl)-1-methyl-1H-indazol-3-yl)piperidine-2,6-dione

To a solution of 4-((1r,4s)-4-(3-bromo-2-methylphenoxy)cyclohexyl)butanal (100 mg, 294.75 mol, 1.0 equiv.) in DCM (1 mL) was added NaOAc (36.27 mg, 442.13 μmol, 1.5 equiv.) and 3-(1-methyl-6-((R)-pyrrolidin-3-yl)-1H-indazol-3-yl)piperidine-2,6-dione (113.10 mg, 324.23 mol, 1.1 equiv., HCl). The mixture was stirred at 25° C. for 0.5 hours. Then, NaBH(OAc)₃ (312.35 mg, 1.47 mmol, 5.0 equiv.) was added into the mixture, and the reaction was stirred at 25° C. for 1.5 hours. The reaction mixture was diluted with DCM (100 mL) and water (100 mL). The layers were separated. The organic layers were combined, dried over anhydrous sodium sulfate, filtered, and concentrated under reduced pressure to give 3-(6-((R)-1-(4-((1r,4s)-4-(3-bromo-2-methylphenoxy)cyclohexyl)butyl)pyrrolidin-3-yl)-1-methyl-1H-indazol-3-yl)piperidine-2,6-dione (150 mg, 235.99 μmol, 80.06% yield) as a yellow oil.

MS (ESI) m/z: 635.4 [M+H]⁺

### Step B. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(4-((3R)-3-(3-(2,6-dioxopiperidin-3-yl)-1-methyl-1H-indazol-6-yl)pyrrolidin-1-yl)butyl)cyclohexyl)oxy)-2-methylphenyl)picolinate

A mixture of 3-(6-((R)-1-(4-((1r,4s)-4-(3-bromo-2-methylphenoxy)cyclohexyl)butyl)pyrrolidin-3-yl)-1-methyl-1H-indazol-3-yl)piperidine-2,6-dione (150 mg, 235.99 μmol, 1.0 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (187.92 mg, 306.78 μmol, 1.3 equiv.), KF (68.55 mg, 1.18 mmol, 27.64 μL, 5 equiv.), H₂O (0.2 mL), and [2-(2-aminophenyl)phenyl]palladium(1⁺); bis(1-adamantyl)-butyl-phosphane; methanesulfonate (34.37 mg, 47.20 μmol, 0.2 equiv.) in dioxane (2 mL) was degassed and purged with N₂ three times. The mixture was stirred at 100° C. for 3 hours under a N₂ atmosphere. The reaction mixture was concentrated under reduced pressure to give a residue. The residue was purified by prep-TLC (SiO₂, DCM:MeOH=15:1) to give tert-butyl 6-(8-(benzo[d]thiazol-2-ylcarbamoyl)-3,4-dihydroisoquinolin-2(1H)-yl)-3-(3-(((1s,4r)-4-(4-((3R)-3-(3-(2,6-dioxopiperidin-3-yl)-1-methyl-1H-indazol-6-yl)pyrrolidin-1-yl)butyl)cyclohexyl)oxy)-2-methylphenyl)picolinate (100 mg, 76.83 μmol, 32.56% yield) as a yellow solid.

MS (ESI) m/z: 1041.7 [M+H]⁺

### Step C. Procedure for Preparation of 6-(8-(benzo[d]thiazol-2-ylcarbamoyl)-3,4-dihydroisoquinolin-2(1H)-yl)-3-(3-(((1s,4r)-4-(4-((3R)-3-(3-(2,6-dioxopiperidin-3-yl)-1-methyl-1H-indazol-6-yl)pyrrolidin-1-yl)butyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1S,4r)-4-(4-((3R*)-3-(3-(2,6-dioxopiperidin-3-yl)-1-methyl-1H-indazol-6-yl)pyrrolidin-1-yl)butyl)cyclohexyl)oxy)-2-methylphenyl)picolinic acid]

To a solution of tert-butyl 6-(8-(benzo[d]thiazol-2-ylcarbamoyl)-3,4-dihydroisoquinolin-2(1H)-yl)-3-(3-(((1s,4r)-4-(4-((3R)-3-(3-(2,6-dioxopiperidin-3-yl)-1-methyl-1H-indazol-6-yl)pyrrolidin-1-yl)butyl)cyclohexyl)oxy)-2-methylphenyl)picolinate (100 mg, 76.83 μmol, 1.0 equiv.) in DCM (1 mL) was added TFA (4.61 g, 40.39 mmol, 3 mL, 525.68 equiv.). The mixture was stirred at 35° C. for 1 hour. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-(4-((3R)-3-(3-(2,6-dioxopiperidin-3-yl)-1-methyl-1H-indazol-6-yl)pyrrolidin-1-yl)butyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1S,4r)-4-(4-((3R*)-3-(3-(2,6-dioxopiperidin-3-yl)-1-methyl-1H-indazol-6-yl)pyrrolidin-1-yl)butyl)cyclohexyl)oxy)-2-methylphenyl)picolinic acid] (47.78 mg, 47.56 μmol, 61.91% yield) as a white solid.

MS (ESI) m/z: 985.7 [M+H]⁺

¹H NMR (400 MHz, DMSO-d₆) δ=13.08-12.48 (m, 1H), 10.88 (s, 1H), 8.03 (d, J=8.0 Hz, 1H), 7.79 (d, J=8.0 Hz, 1H), 7.70 (d, J=8.4 Hz, 1H), 7.62 (d, J=7.2 Hz, 1H), 7.55 (s, 1H), 7.51-7.42 (m, 3H), 7.40-7.32 (m, 2H), 7.13 (d, J=8.4 Hz, 1H), 7.10-7.04 (m, 1H), 6.99-6.89 (m, 2H), 6.61 (d, J=8.0 Hz, 1H), 4.98 (s, 2H), 4.39-4.32 (m, 1H), 4.24-4.15 (m, 1H), 3.99 (s, 3H), 3.91 (t, J=5.6 Hz, 2H), 3.80-3.57 (m, 2H), 3.23-3.10 (m, 4H), 3.02 (t, J=5.6 Hz, 2H), 2.71-2.65 (m, 1H), 2.64-2.60 (m, 1H), 2.35-2.30 (m, 1H), 2.23-2.01 (m, 5H), 1.87 (s, 3H), 1.79-1.77 (m, 2H), 1.64 (s, 2H), 1.41-1.32 (m, 4H), 1.25-1.23 (m, 4H), 1.12-1.00 (m, 2H)

### Example 266. Preparation of Compound 332b

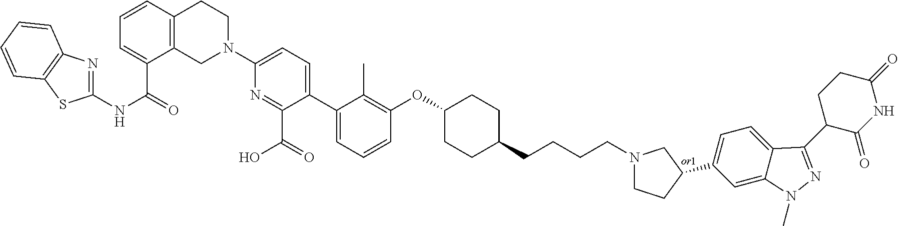 [SMILES: Cc1c(O[C@H]2CC[C@H](CCCCN3CC[C@@H](c4ccc5c(C6CCC(=O)NC6=O)nn(C)c5c4)C3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-(4-((3S)-3-(3-(2,6-dioxopiperidin-3-yl)-1-methyl-1H-indazol-6-yl)pyrrolidin-1-yl)butyl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1R,4r)-4-(4-((3S*)-3-(3-(2,6-dioxopiperidin-3-yl)-1-methyl-1H-indazol-6-yl)pyrrolidin-1-yl)butyl)cyclohexyl)oxy)-2-methylphenyl)picolinic acid]

### Step A. Procedure for Preparation of 3-(6-((S)-1-(4-((1r,4r)-4-(3-bromo-2-methylphenoxy)cyclohexyl)butyl)pyrrolidin-3-yl)-1-methyl-1H-indazol-3-yl)piperidine-2,6-dione

To a solution of 4-((1r,4s)-4-(3-bromo-2-methylphenoxy)cyclohexyl)butanal (100 mg, 294.75 μmol, 1.0 equiv.) in DCM (2 mL) was added 3-(1-methyl-6-((S)-pyrrolidin-3-yl)-1H-indazol-3-yl)piperidine-2,6-dione (101.28 mg, 324.23 μmol, 1.1 equiv.), NaOAc (36.27 mg, 442.13 μmol, 1.5 equiv.), and NaBH(OAc)₃ (312.35 mg, 1.47 mmol, 5.0 equiv.). The mixture was stirred at 25° C. for 2 hours. The reaction mixture was concentrated under reduced pressure to give a residue. The residue was diluted with H₂O (20 mL) and extracted with DCM (30 mL×2). The combined organic layers were washed with DCM (20 mL), dried over Na₂SO₄, filtered, and concentrated under reduced pressure to give 3-(6-((S)-1-(4-((1r,4r)-4-(3-bromo-2-methylphenoxy)cyclohexyl)butyl)pyrrolidin-3-yl)-1-methyl-1H-indazol-3-yl)piperidine-2,6-dione (150 mg, 235.99 μmol, 80.06% yield) as a yellow solid.

MS (ESI) m/z: 635.4 [M+H]⁺.

### Step B. Procedure for Preparation of tert-butyl 6-(8-(benzo[d]thiazol-2-ylcarbamoyl)-3,4-dihydroisoquinolin-2(1H)-yl)-3-(3-(((1r,4r)-4-(4-((3S)-3-(3-(2,6-dioxopiperidin-3-yl)-1-methyl-1H-indazol-6-yl)pyrrolidin-1-yl)butyl)cyclohexyl)oxy)-2-methylphenyl)picolinate

A mixture of 3-(6-((S)-1-(4-((1r,4r)-4-(3-bromo-2-methylphenoxy)cyclohexyl)butyl)pyrrolidin-3-yl)-1-methyl-1H-indazol-3-yl)piperidine-2,6-dione (150 mg, 235.99 μmol, 1.0 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (173.46 mg, 283.18 μmol, 1.2 equiv.), KF (27.42 mg, 471.97 mol, 11.06 μL, 2.0 equiv.), [2-(2-aminophenyl)phenyl]palladium(1⁺); bis(1-adamantyl)-butyl-phosphane; methanesulfonate (34.37 mg, 47.20 μmol, 0.2 equiv.), and H₂O (1 mL) in dioxane (2 mL) was degassed and purged with N₂ three times. The mixture was stirred at 100° C. for 3 hours under N₂ atmosphere. The reaction mixture was concentrated under reduced pressure to give a residue. The residue was purified by prep-TLC (SiO₂, petroleum ether/ethyl acetate=0/1 to DCM/MeOH=15/1) to give tert-butyl 6-(8-(benzo[d]thiazol-2-ylcarbamoyl)-3,4-dihydroisoquinolin-2(1H)-yl)-3-(3-(((1r,4r)-4-(4-((3S)-3-(3-(2,6-dioxopiperidin-3-yl)-1-methyl-1H-indazol-6-yl)pyrrolidin-1-yl)butyl)cyclohexyl)oxy)-2-methylphenyl)picolinate (100 mg, 96.03 μmol, 40.69% yield) as a yellow solid.

MS (ESI) m/z: 1041.7 [M+H]⁺

### Step C. Procedure for Preparation of 6-(8-(benzo[d]thiazol-2-ylcarbamoyl)-3,4-dihydroisoquinolin-2(1H)-yl)-3-(3-(((1r,4r)-4-(4-((3S)-3-(3-(2,6-dioxopiperidin-3-yl)-1-methyl-1H-indazol-6-yl)pyrrolidin-1-yl)butyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1R,4r)-4-(4-((3S*)-3-(3-(2,6-dioxopiperidin-3-yl)-1-methyl-1H-indazol-6-yl)pyrrolidin-1-yl)butyl)cyclohexyl)oxy)-2-methylphenyl)picolinic acid]

To a solution of tert-butyl 6-(8-(benzo[d]thiazol-2-ylcarbamoyl)-3,4-dihydroisoquinolin-2(1H)-yl)-3-(3-(((1r,4r)-4-(4-((3S)-3-(3-(2,6-dioxopiperidin-3-yl)-1-methyl-1H-indazol-6-yl)pyrrolidin-1-yl)butyl)cyclohexyl)oxy)-2-methylphenyl)picolinate (100 mg, 96.03 μmol, 1.0 equiv.) in TFA (2 mL) was added DCM (2 mL). The mixture was stirred at 40° C. for 1 hour. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1r,4r)-4-(4-((3S)-3-(3-(2,6-dioxopiperidin-3-yl)-1-methyl-1H-indazol-6-yl)pyrrolidin-1-yl)butyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1R,4r)-4-(4-((3S*)-3-(3-(2,6-dioxopiperidin-3-yl)-1-methyl-1H-indazol-6-yl)pyrrolidin-1-yl)butyl)cyclohexyl)oxy)-2-methylphenyl)picolinic acid](34.50 mg, 32.95 μmol, 34.31% yield) as a white solid.

MS (ESI) m/z: 985.7 [M+H]⁺.

¹H NMR (400 MHz, DMSO-d₆) δ=10.89 (s, 1H), 8.03 (d, J=8.0 Hz, 1H), 7.79 (d, J=8.0 Hz, 1H), 7.69 (d, J=8.4 Hz, 1H), 7.63 (d, J=7.6 Hz, 1H), 7.54 (s, 1H), 7.50-7.43 (m, 3H), 7.40-7.33 (m, 2H), 7.14-7.05 (m, 2H), 6.99-6.89 (m, 2H), 6.65-6.60 (m, 1H), 4.98 (s, 2H), 4.36 (dd, J=5.2, 9.6 Hz, 1H), 4.25-4.15 (m, 1H), 3.99 (s, 3H), 3.92 (t, J=5.6 Hz, 2H), 3.65-3.54 (m, 2H), 3.04-3.02 (m, 4H), 2.70-2.55 (m, 4H), 2.44-2.30 (m, 4H), 2.25-2.12 (m, 2H), 2.12-2.04 (m, 3H), 1.80-1.78 (m, 2H), 1.64-1.56 (m, 2H), 1.38-1.36 (m, 4H), 1.26-1.24 (m, 4H), 1.12-1.02 (m, 2H).

### Example 267. Preparation of Compound 333a

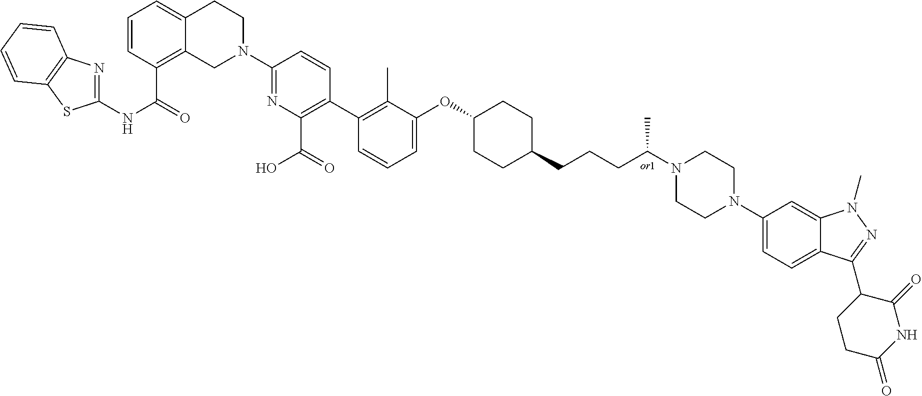 [SMILES: Cc1c(O[C@H]2CC[C@H](CCC[C@H](C)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-((4S)-4-(4-(3-(2,6-dioxopiperidin-3-yl)-1-methyl-1H-indazol-6-yl)piperazin-1-yl)pentyl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1R,4r)-4-((4S*)-4-(4-(3-(2,6-dioxopiperidin-3-yl)-1-methyl-1H-indazol-6-yl)piperazin-1-yl)pentyl)cyclohexyl)oxy)-2-methylphenyl)picolinic acid]

### Step A. Procedure for Preparation of 5-((1r,4s)-4-(3-bromo-2-methylphenoxy)cyclohexyl)pentan-2-ol

To a solution of 4-((1r,4s)-4-(3-bromo-2-methylphenoxy)cyclohexyl)butanal (2.5 g, 7.37 mmol, 1.0 equiv.) in THE (25 mL) was added MeMgBr (3 M, 4.91 mL, 2.0 equiv.) at −70° C. The mixture was stirred at 25° C. for 12 hours at N₂ atmosphere. The mixture was quenched with aq. NH₄Cl (200 mL) and extracted with EtOAc (50 mL×3). The combined organic phases were washed with brine (100 mL×1), dried over Na₂SO₄, filtered, and concentrated under reduced pressure to give a residue. The residue was purified by flash silica gel chromatography (0˜13% ethyl acetate/petroleum ether) to give 5-((1r,4s)-4-(3-bromo-2-methylphenoxy)cyclohexyl)pentan-2-ol (2.0 g, 5.07 mmol, 68.75% yield) as a brown oil.

¹H NMR (400 MHz, DMSO-d₆) δ=7.13 (d, J=8.0 Hz, 1H), 7.06 (t, J=8.0 Hz, 1H), 7.01 (d, J=8.0 Hz, 1H), 4.28 (d, J=4.0 Hz, 1H), 4.26-4.17 (m, 1H), 3.61-3.51 (m, 1H), 2.20 (s, 3H), 2.04 (d, J=10.4 Hz, 2H), 1.76 (d, J=12.4 Hz, 2H), 1.38-1.23 (m, 7H), 1.20-1.12 (m, 2H), 1.03 (d, J=6.0 Hz, 5H)

### Step B. Procedure for Preparation of 5-((1r,4s)-4-(3-bromo-2-methylphenoxy)cyclohexyl)pentan-2-one

To a solution of 5-((1r,4s)-4-(3-bromo-2-methylphenoxy)cyclohexyl)pentan-2-ol (2.3 g, 6.47 mmol, 1.0 equiv.) in DCM (20 mL) was added DMP (5.49 g, 12.95 mmol, 4.01 mL, 2.0 equiv.) at 0° C. The mixture was stirred at 25° C. for 12 hours at N₂ atmosphere. The reaction mixture was filtered and concentrated under reduced pressure to give a residue. The residue was purified by flash silica gel chromatography (0˜13% ethyl acetate/petroleum ether) to give 5-((1r,4s)-4-(3-bromo-2-methylphenoxy)cyclohexyl)pentan-2-one (2.0 g, 5.66 mmol, 87.45% yield) as a white oil.

¹H NMR (400 MHz, DMSO-d₆) δ=7.13 (d, J=8.0 Hz, 1H), 7.07 (t, J=8.0 Hz, 1H), 7.01 (d, J=8.0 Hz, 1H), 4.30-4.17 (m, 1H), 2.40 (t, J=7.2 Hz, 2H), 2.21 (s, 3H), 2.09-1.99 (m, 5H), 1.75 (d, J=12.4 Hz, 2H), 1.47 (td, J=7.2, 15.1 Hz, 2H), 1.41-1.28 (m, 2H), 1.24 (d, J=5.6 Hz, 1H), 1.18-1.09 (m, 2H), 1.09-0.96 (m, 2H)

### Step C. Procedure for Preparation of tert-butyl 4-(5-((1r,4s)-4-(3-bromo-2-methylphenoxy)cyclohexyl)pentan-2-yl)piperazine-1-carboxylate

A mixture of 5-((1r,4s)-4-(3-bromo-2-methylphenoxy)cyclohexyl)pentan-2-one (500 mg, 1.42 mmol, 1.0 equiv.) and tert-butyl piperazine-1-carboxylate (289.95 mg, 1.56 mmol, 1.1 equiv.) in DCM (10 mL) was stirred for 2 hours. NaBH(OAc)₃ (1.50 g, 7.08 mmol, 5.0 equiv.) was added into the mixture, and then the mixture was stirred at 25° C. for 12 hours under N₂ atmosphere. The reaction mixture was quenched with H₂O (50 mL) and extracted with EtOAc (50 mL×3). The combined organic layers were washed with brine (50 mL×1), dried over Na₂SO₄, filtered, and concentrated under reduced pressure to give a residue. The residue was purified by flash silica gel chromatography (0˜20% ethyl acetate/petroleum ether) to give tert-butyl 4-(5-((1r,4s)-4-(3-bromo-2-methylphenoxy)cyclohexyl)pentan-2-yl)piperazine-1-carboxylate (0.5 g, 926.38 μmol, 65.46% yield) as a white solid.

### Step D. Procedure for Preparation of tert-butyl 4-((S)-5-((1s,4s)-4-(3-bromo-2-methylphenoxy)cyclohexyl)pentan-2-yl)piperazine-1-carboxylate

The stereoisomers of tert-butyl 4-(5-((1r,4s)-4-(3-bromo-2-methylphenoxy)cyclohexyl)pentan-2-yl)piperazine-1-carboxylate (1.0 g, 1.91 mmol, 1.0 equiv.) were separated by SFC to give tert-butyl 4-((S)-5-((1s,4s)-4-(3-bromo-2-methylphenoxy)cyclohexyl)pentan-2-yl)piperazine-1-carboxylate (490 mg, 935.93 μmol, 49.00% yield) as a yellow oil.

### Step E. Procedure for Preparation of 1-((S)-5-((1s, 4s)-4-(3-bromo-2-methylphenoxy)cyclohexyl)pentan-2-yl)piperazine

A mixture of tert-butyl 4-((S)-5-((1s,4s)-4-(3-bromo-2-methylphenoxy)cyclohexyl)pentan-2-yl)piperazine-1-carboxylate (600 mg, 1.15 mmol, 1.0 equiv.) in HCl/dioxane (10 mL) was stirred at 25° C. for 1 hour. The reaction mixture was concentrated under reduced pressure to give a residue. The residue was purified by reverse-phase HPLC (with 0.1% formic acid) to give 1-((S)-5-((is, 4s)-4-(3-bromo-2-methylphenoxy)cyclohexyl)pentan-2-yl)piperazine (400 mg, 925.78 mol, 80.78% yield) as a brown oil.

MS (ESI) m/z: 423.3 [M+H]⁺

### Step F. Procedure for Preparation of [3-(2,6-dibenzyloxy-3-pyridyl)-1-methyl-indazol-6-yl]boronic acid

To a solution of 6-bromo-3-(2,6-dibenzyloxy-3-pyridyl)-1-methyl-indazole (2.0 g, 4.00 mmol, 1.0 equiv.) and triisopropyl borate (2.26 g, 11.99 mmol, 2.76 mL, 3.0 equiv.) in THE (5 mL) was added n-BuLi (2.5 M, 3.20 mL, 2.0 equiv.) at −78° C. The mixture was stirred at −78° C. for 1 hour under N₂ atmosphere. The reaction mixture was diluted with NH₄Cl (100 mL) and extracted with EtOAc (50 mL×3). The combined organic layers were washed with brine (50 mL×1), dried over Na₂SO₄, filtered, and concentrated under reduced pressure to give a residue. The residue was purified by flash silica gel chromatography (0˜2% dichloromethane:methanol) to give [3-(2,6-dibenzyloxy-3-pyridyl)-1-methyl-indazol-6-yl]boronic acid (1.9 g, 3.47 mmol, 86.84% yield,) as a white solid.

MS (ESI) m/z: 466.3 [M+H]⁺

### Step G. Procedure for Preparation of 3-(2,6-bis(benzyloxy)pyridin-3-yl)-6-(4-((S)-5-((1r,4s)-4-(3-bromo-2-methylphenoxy)cyclohexyl)pentan-2-yl)piperazin-1-yl)-1-methyl-1H-indazole

A mixture of 1-((S)-5-((is, 4s)-4-(3-bromo-2-methylphenoxy)cyclohexyl)pentan-2-yl)piperazine (50 mg, 118.08 μmol, 1.0 equiv.), [3-(2,6-dibenzyloxy-3-pyridyl)-1-methyl-indazol-6-yl]boronic acid (65.93 mg, 141.70 μmol, 1.2 equiv.), Cu(OAc)₂ (4.29 mg, 23.62 μmol, 0.2 equiv.), TEA (35.85 mg, 354.25 μmol, 49.31 μL, 3 equiv.), and 4A MS (1 g, 118.08 μmol, 1.0 equiv.) in DCE (10 mL) was degassed and purged with O₂ three times. The mixture was stirred at 25° C. for 12 hours under O₂ atmosphere. The reaction mixture was filtered and concentrated under reduced pressure to give a residue. The residue was purified by prep-TLC (SiO₂, DCM:MeOH=20:1) to give 3-(2,6-bis(benzyloxy)pyridin-3-yl)-6-(4-((S)-5-((1r,4s)-4-(3-bromo-2-methylphenoxy)cyclohexyl)pentan-2-yl)piperazin-1-yl)-1-methyl-1H-indazole (100 mg, 58.13 μmol, 12.31% yield) as a yellow oil.

MS (ESI) m/z: 842.7 [M+H]⁺

### Step H. Procedure for Preparation of 3-(2,6-bis(benzyloxy)pyridin-3-yl)-1-methyl-6-(4-((S)-5-((1r,4R)-4-(2-methyl-3-(4,4,5,5-tetramethyl-1,3,2-dioxaborolan-2-yl)phenoxy)cyclohexyl)pentan-2-yl)piperazin-1-yl)-1H-indazole

A mixture of 3-(2,6-bis(benzyloxy)pyridin-3-yl)-6-(4-((S)-5-((1r,4s)-4-(3-bromo-2-methylphenoxy)cyclohexyl)pentan-2-yl)piperazin-1-yl)-1-methyl-1H-indazole (100 mg, 118.64 mol, 1.0 equiv.), B₂pin₂ (60.25 mg, 237.28 μmol, 2.0 equiv.), KOAc (34.93 mg, 355.91 μmol, 3.0 equiv.), and Pd(dppf)Cl₂ (8.68 mg, 11.86 μmol, 0.1 equiv.) in DMSO (10 mL) was degassed and purged with N₂ three times. The mixture was stirred at 100° C. for 12 hours under N₂ atmosphere. The reaction mixture was diluted with H₂O (50 mL) and extracted with EtOAc (50 mL×3). The combined organic layers were washed with brine (50 mL×1), dried over Na₂SO₄, filtered, and concentrated under reduced pressure to give a residue. The residue was purified by prep-TLC (SiO₂, DCM:MeOH=20:1) to give 3-(2,6-bis(benzyloxy)pyridin-3-yl)-1-methyl-6-(4-((S)-5-((1r,4R)-4-(2-methyl-3-(4,4,5,5-tetramethyl-1,3,2-dioxaborolan-2-yl)phenoxy)cyclohexyl)pentan-2-yl)piperazin-1-yl)-1H-indazole (70 mg, 39.33 μmol, 33.15% yield) as a yellow oil.

MS (ESI) m/z: 890.8 [M+H]⁺

### Step I. Procedure for preparation of 3-(1-methyl-6-(4-((S)-5-((1r,4r)-4-(2-methyl-3-(4,4,5,5-tetramethyl-1,3,2-dioxaborolan-2-yl)phenoxy)cyclohexyl)pentan-2-yl)piperazin-1-yl)-1H-indazol-3-yl)piperidine-2,6-dione

To a solution of 3-(2,6-bis(benzyloxy)pyridin-3-yl)-1-methyl-6-(4-((S)-5-((1r,4R)-4-(2-methyl-3-(4,4,5,5-tetramethyl-1,3,2-dioxaborolan-2-yl)phenoxy)cyclohexyl)pentan-2-yl)piperazin-1-yl)-1H-indazole (70 mg, 78.65 μmol, 1.0 equiv.) in THE (5 mL) and EtOH (5 mL) was added Pd/C (8.37 mg, 7.87 μmol, 10% purity, 0.1 equiv.) and Pd(OH)₂ (5.52 mg, 7.87 μmol, 20% purity, 0.1 equiv.) under N₂ atmosphere. The suspension was degassed and purged with H₂ three times. The mixture was stirred under H₂ (15 Psi) at 25° C. for 12 hours. The reaction mixture was then filtered and concentrated under reduced pressure to give 3-(1-methyl-6-(4-((S)-5-((1r,4r)-4-(2-methyl-3-(4,4,5,5-tetramethyl-1,3,2-dioxaborolan-2-yl)phenoxy)cyclohexyl)pentan-2-yl)piperazin-1-yl)-1H-indazol-3-yl)piperidine-2,6-dione (80 mg, 50.58 μmol, 64.31% yield) as a yellow oil.

MS (ESI) m/z: 712.7 [M+H]⁺

### Step J. Procedure for Preparation of tert-butyl 6-(8-(benzo[d]thiazol-2-ylcarbamoyl)-3,4-dihydroisoquinolin-2(1H)-yl)-3-(3-(((1r,4r)-4-((4S)-4-(4-(3-(2,6-dioxopiperidin-3-yl)-1-methyl-1H-indazol-6-yl)piperazin-1-yl)pentyl)cyclohexyl)oxy)-2-methylphenyl)picolinate

A mixture of 3-(1-methyl-6-(4-((S)-5-((1r,4r)-4-(2-methyl-3-(4,4,5,5-tetramethyl-1,3,2-dioxaborolan-2-yl)phenoxy)cyclohexyl)pentan-2-yl)piperazin-1-yl)-1H-indazol-3-yl)piperidine-2,6-dione (80 mg, 112.40 μmol, 1.0 equiv.), tert-butyl 6-[8-(1,3-benzothiazol-2-ylcarbamoyl)-3,4-dihydro-1H-isoquinolin-2-yl]-3-bromo-pyridine-2-carboxylate (76.27 mg, 134.88 μmol, 1.2 equiv.), [2-(2-aminophenyl)phenyl]palladium(1⁺); bis(1-adamantyl)-butyl-phosphane; methanesulfonate (8.19 mg, 11.24 μmol, 0.1 equiv.), and KF (19.59 mg, 337.20 μmol, 7.90 μL, 3.0 equiv.) in dioxane (10 mL) and H₂O (1 mL) was degassed and purged with N₂ three times. The mixture was stirred at 100° C. for 12 hours under N₂ atmosphere. The reaction mixture was filtered and concentrated under reduced pressure to give a residue. The residue was purified by prep-TLC (SiO₂, DCM:MeOH=20:1) to give tert-butyl 6-(8-(benzo[d]thiazol-2-ylcarbamoyl)-3,4-dihydroisoquinolin-2(1H)-yl)-3-(3-(((1r,4r)-4-((4S)-4-(4-(3-(2,6-dioxopiperidin-3-yl)-1-methyl-1H-indazol-6-yl)piperazin-1-yl)pentyl)cyclohexyl)oxy)-2-methylphenyl)picolinate (30 mg, 19.62 μmol, 17.46% yield) as a yellow oil.

MS (ESI) m/z: 1070.5 [M+H]⁺

### Step K. Procedure for Preparation of 6-(8-(benzo[d]thiazol-2-ylcarbamoyl)-3,4-dihydroisoquinolin-2(1H)-yl)-3-(3-(((1r,4r)-4-((4S)-4-(4-(3-(2,6-dioxopiperidin-3-yl)-1-methyl-1H-indazol-6-yl)piperazin-1-yl)pentyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1R,4r)-4-((4S*)-4-(4-(3-(2,6-dioxopiperidin-3-yl)-1-methyl-1H-indazol-6-yl)piperazin-1-yl)pentyl)cyclohexyl)oxy)-2-methylphenyl)picolinic acid]

A mixture of tert-butyl 6-(8-(benzo[d]thiazol-2-ylcarbamoyl)-3,4-dihydroisoquinolin-2(1H)-yl)-3-(3-(((1r,4r)-4-((4S)-4-(4-(3-(2,6-dioxopiperidin-3-yl)-1-methyl-1H-indazol-6-yl)piperazin-1-yl)pentyl)cyclohexyl)oxy)-2-methylphenyl)picolinate (30 mg, 28.03 μmol, 1.0 equiv.) in TFA (2 mL) and DCM (2 mL) was stirred at 25° C. for 12 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1r,4r)-4-((4S)-4-(4-(3-(2,6-dioxopiperidin-3-yl)-1-methyl-1H-indazol-6-yl)piperazin-1-yl)pentyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1R,4r)-4-((4S*)-4-(4-(3-(2,6-dioxopiperidin-3-yl)-1-methyl-1H-indazol-6-yl)piperazin-1-yl)pentyl)cyclohexyl)oxy)-2-methylphenyl)picolinic acid] (14.11 mg, 13.56 μmol, 48.39% yield) as a yellow solid.

MS (ESI) m/z: 1014.5 [M+H]⁺

¹H NMR (400 MHz, DMSO-d₆) δ=10.84 (s, 1H), 8.02 (d, J=8.0 Hz, 1H), 7.78 (d, J=8.4 Hz, 1H), 7.62 (d, J=7.2 Hz, 1H), 7.51-7.32 (m, 6H), 7.10-7.01 (m, 1H), 6.96-6.86 (m, 3H), 6.84-6.79 (m, 1H), 6.62 (d, J=7.6 Hz, 1H), 4.97 (s, 2H), 4.28-4.12 (m, 2H), 3.93-3.86 (m, 5H), 3.19 (s, 4H), 3.02 (t, J=5.6 Hz, 2H), 2.65-2.55 (m, 6H), 2.35-2.27 (m, 2H), 2.19-2.13 (m, 1H), 2.10-2.04 (m, 2H), 1.87 (s, 3H), 1.81-1.74 (m, 2H), 1.52-1.46 (m, 1H), 1.38-1.31 (m, 4H), 1.28 (s, 2H), 1.23 (s, 2H), 1.09-1.01 (m, 2H), 0.95 (d, J=6.4 Hz, 3H)

### Example 268. Preparation of Compound 319a

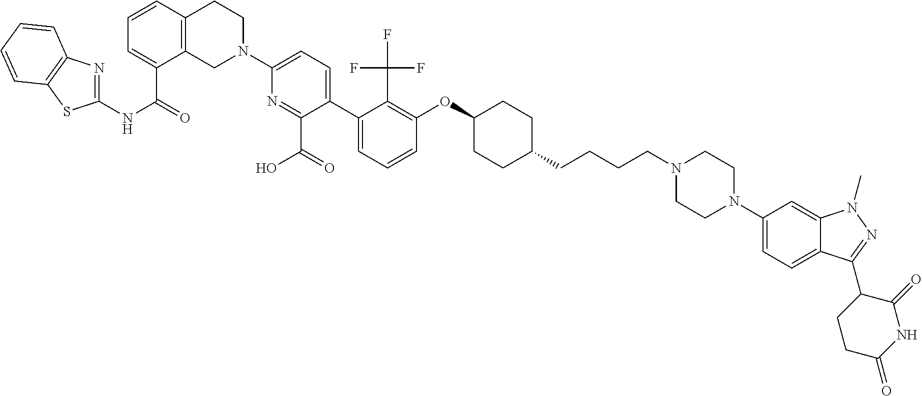 [SMILES: Cn1nc(C2CCC(=O)NC2=O)c2ccc(N3CCN(CCCC[C@H]4CC[C@H](Oc5cccc(-c6ccc(N7CCc8cccc(C(=O)Nc9nc%10ccccc%10s9)c8C7)nc6C(=O)O)c5C(F)(F)F)CC4)CC3)cc21]

### 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)butyl)cyclohexyl)oxy)-2-(trifluoromethyl)phenyl)picolinic acid

### Step A. Procedure for Preparation of methyl (1r,4r)-4-(3-bromo-2-(trifluoromethyl)phenoxy)cyclohexane-1-carboxylate

A mixture of methyl (1s,4s)-4-hydroxycyclohexane-1-carboxylate (1.58 g, 9.96 mmol, 1.2 equiv.), 3-bromo-2-(trifluoromethyl)phenol (2 g, 8.30 mmol, 1.0 equiv.), and 2-(tributyl-phosphanylidene)acetonitrile (2.60 g, 10.79 mmol, 1.3 equiv.) in toluene (40 mL) was degassed and purged with N₂ three times. The mixture was stirred at 120° C. for 12 hours under N₂ atmosphere. The reaction mixture was filtered and concentrated under reduced pressure to give a residue. The residue was purified by column chromatography (SiO₂, petroleum ether/ethyl acetate=100/1) to give methyl (1r,4r)-4-(3-bromo-2-(trifluoromethyl)phenoxy)cyclohexane-1-carboxylate (2 g, 2.62 mmol, 31.61% yield, 50% purity) as a yellow oil.

¹H NMR (400 MHz, DMSO-d₆) δ=7.49-7.42 (m, 1H), 7.41-7.34 (m, 2H), 4.60-4.46 (m, 1H), 3.60 (s, 3H), 2.45-2.40 (m, 1H), 2.06-2.00 (m, 2H), 1.96-1.89 (m, 2H), 1.58-1.43 (m, 4H).

### Step B. Procedure for Preparation of ((1r,4r)-4-(3-bromo-2-(trifluoromethyl)phenoxy)cyclohexyl)methanol

A mixture of methyl (1r,4r)-4-(3-bromo-2-(trifluoromethyl)phenoxy)cyclohexane-1-carboxylate (2 g, 2.62 mmol, 1.0 equiv.) in THE (40 mL) was degassed and purged with N₂ three times. To this mixture was added LiAlH₄ (2.5 M, 1.05 mL, 1.0 equiv.). The reaction mixture was stirred at 0° C. for 2 hours under N₂ atmosphere. The reaction mixture was quenched with aqueous Na₂SO₄ (0.4 mL) at 0° C. The mixture was diluted with ethyl acetate (60 mL), filtered, and concentrated under reduced pressure to give a residue. The residue was purified by flash silica gel chromatography (0˜19% ethyl acetate/petroleum ether) to give ((1r,4r)-4-(3-bromo-2-(trifluoromethyl)phenoxy)cyclohexyl)methanol (450 mg, 1.15 mmol, 43.71% yield) as a colorless oil.

¹H NMR (400 MHz, DMSO-d₆) δ=7.48-7.41 (m, 1H), 7.39-7.32 (m, 2H), 4.48-4.39 (m, 2H), 3.23 (t, J=5.6 Hz, 2H), 2.05 (d, J=9.2 Hz, 2H), 1.84-1.72 (m, 2H), 1.40-1.28 (m, 3H), 1.12-0.99 (m, 2H).

### Step C. Procedure for Preparation of (1r,4r)-4-(3-bromo-2-(trifluoromethyl)phenoxy)cyclohexane-1-carbaldehyde

A solution of oxalyl dichloride (323.45 mg, 2.55 mmol, 223.07 μL, 2.0 equiv.) in DCM (40 mL) was cooled to −78° C., and DMSO (398.22 mg, 5.10 mmol, 398.22 μL, 4.0 equiv.) was added dropwise. The mixture was stirred at −78° C. for 2 hours under N₂ atmosphere. Then ((1r,4r)-4-(3-bromo-2-(trifluoromethyl)phenoxy)cyclohexyl)methanol (450 mg, 1.27 mmol, 1.0 equiv.) was added dropwise. The mixture was stirred at −78° C. for 1 hour under N₂ atmosphere. TEA (773.59 mg, 7.64 mmol, 1.06 mL, 6 equiv.) was added dropwise to the mixture at −78° C., and the mixture was warmed to 25° C. and stirred for 1 hour under N₂ atmosphere. The reaction mixture was quenched with water (40 mL) at 0° C. and extracted with ethyl acetate (50 mL×3). The combined organic layers were washed with brine (20 mL×3), dried over Na₂SO₄, filtered, and concentrated under reduced pressure to give a residue. The residue was purified by flash silica gel chromatography (0˜5% ethyl acetate/petroleum ether) to give (1r,4r)-4-(3-bromo-2-(trifluoromethyl)phenoxy)cyclohexane-1-carbaldehyde (400 mg, 1.03 mmol, 80.46% yield) as a red oil.

¹H NMR (400 MHz, DMSO-d₆) δ=9.61 (s, 1H), 7.45 (t, J=8.4 Hz, 1H), 7.39-7.34 (m, 2H), 4.57-4.48 (m, 1H), 2.42-2.35 (m, 1H), 2.04-1.90 (m, 4H), 1.55-1.40 (m, 4H).

### Step D. Procedure for Preparation of 1-(((1r,4r)-4-((E)-4-(benzyloxy)but-1-en-1-yl)cyclohexyl)oxy)-3-bromo-2-(trifluoromethyl)benzene

To a solution of (3-(benzyloxy)propyl)triphenylphosphonium bromide (1.12 g, 2.28 mmol, 2.0 equiv.) in THE (30 mL) was added LiHMDS (1 M, 3.42 mL, 3.0 equiv.) at 0° C. The reaction mixture was stirred for 1 hour. Afterwards, (1r,4r)-4-(3-bromo-2-(trifluoromethyl)phenoxy)cyclohexane-1-carbaldehyde (400 mg, 1.14 mmol, 1.0 equiv.) was added, and the mixture was stirred at 0° C. for 3 hours under N₂ atmosphere. The reaction mixture was quenched with aqueous NH₄Cl (30 mL) at 0° C. and extracted with ethyl acetate (40 mL). The combined organic layers were washed with brine (20 mL), dried over sodium sulfate, filtered, and concentrated under reduced pressure to give a residue. The residue was purified by flash silica gel chromatography (0˜3% ethyl acetate/petroleum ether) to give 1-(((1r,4r)-4-((E)-4-(benzyloxy)but-1-en-1-yl)cyclohexyl)oxy)-3-bromo-2-(trifluoromethyl)benzene (470 mg, 875.12 mol, 76.77% yield) as a colorless oil.

¹H NMR (400 MHz, DMSO-d₆) δ=7.44 (d, J=7.6 Hz, 1H), 7.40-7.24 (m, 7H), 5.52-5.18 (m, 2H), 4.55-4.36 (m, 3H), 3.47-3.37 (m, 2H), 2.36-2.27 (m, 3H), 2.03 (d, J=9.6 Hz, 2H), 1.63 (d, J=12.4 Hz, 2H), 1.51-1.34 (m, 2H), 1.31-1.12 (m, 2H).

### Step E. Procedure for Preparation of 1-(((1s,4r)-4-(4-(benzyloxy)butyl)cyclohexyl)oxy)-3-bromo-2-(trifluoromethyl)benzene

To a solution of 1-(((1r,4r)-4-((E)-4-(benzyloxy)but-1-en-1-yl)cyclohexyl)oxy)-3-bromo-2-(trifluoromethyl)benzene (470 mg, 972.36 μmol, 1.0 equiv.) in EtOAc (5 mL) was added Rh/Al₂O₃ (235.00 mg, 2.28 mmol, 2.35 equiv.) under N₂ atmosphere. The suspension was degassed and purged with H₂ three times. The mixture was stirred under H₂ (15 Psi) at 25° C. for 12 hours. The reaction solution was filtered through the diatom layer under a moderate N₂ atmosphere. The reaction mixture was filtered and concentrated under reduced pressure to give 1-(((1s,4r)-4-(4-(benzyloxy)butyl)cyclohexyl)oxy)-3-bromo-2-(trifluoromethyl)benzene (460 mg, crude) as a colorless oil.

¹H NMR (400 MHz, DMSO-d₆) δ=7.48-7.41 (m, 1H), 7.38-7.27 (m, 7H), 4.44 (s, 3H), 3.43-3.39 (m, 2H), 2.03 (d, J=9.2 Hz, 2H), 1.75 (d, J=12.0 Hz, 2H), 1.55-1.49 (m, 2H), 1.36-1.29 (m, 4H), 1.24-1.14 (m, 3H), 1.10-0.94 (m, 2H).

### Step F. Procedure for Preparation of 4-((1r,4s)-4-(3-bromo-2-(trifluoromethyl)phenoxy)cyclohexyl)butan-1-ol

To a solution of 1-(((1s,4r)-4-(4-(benzyloxy)butyl)cyclohexyl)oxy)-3-bromo-2-(trifluoromethyl)benzene (460 mg, 947.72 μmol, 1.0 equiv.) in DCM (10 mL) was added TMSI (568.90 mg, 2.84 mmol, 387.00 μL, 3.0 equiv.) at 0° C. The mixture was stirred at 25° C. for 2 hours under N₂ atmosphere. The reaction mixture was quenched with water (10 mL) and extracted with dichloromethane (10 mL×3). The combined organic layers were washed with brine (10 mL×3), dried over Na₂SO₄, filtered, and concentrated under reduced pressure to give a residue. The residue was purified by flash silica gel chromatography (0˜20% ethyl acetate/petroleum ether) to give compound 4-((1r,4s)-4-(3-bromo-2-(trifluoromethyl)phenoxy)cyclohexyl)butan-1-ol (220 mg, 500.94 μmol, 52.86% yield) as a colorless oil.

¹H NMR (400 MHz, DMSO-d₆) δ=7.43 (d, J=8.0 Hz, 1H), 7.39-7.27 (m, 2H), 4.44 (t, J=10.4 Hz, 1H), 4.32 (t, J=4.8 Hz, 1H), 3.43-3.34 (m, 2H), 2.12-1.93 (m, 2H), 1.76 (d, J=12.4 Hz, 2H), 1.46-1.34 (m, 3H), 1.34-1.22 (m, 4H), 1.21-1.13 (m, 2H), 1.10-0.95 (m, 2H).

### Step G. Procedure for Preparation of 4-((1r,4s)-4-(3-bromo-2-(trifluoromethyl)phenoxy)cyclohexyl)butanal

To a solution of oxalyl dichloride (141.30 mg, 1.11 mmol, 97.45 μL, 2.0 equiv.) in DCM (30 mL) cooled to −78° C., was added DMSO (173.95 mg, 2.23 mmol, 173.95 μL, 4.0 equiv.) dropwise. The mixture was stirred at −78° C. for 2 hours under N₂ atmosphere. Then, 4-((1r,4s)-4-(3-bromo-2-(trifluoromethyl)phenoxy)cyclohexyl)butan-1-ol (220 mg, 556.60 μmol, 1.0 equiv.) was added dropwise, and the mixture was stirred at −78° C. for 1 hour under N₂ atmosphere. TEA (337.94 mg, 3.34 mmol, 464.84 μL, 6.0 equiv.) was added dropwise to the mixture at −78° C., and the mixture was warmed to 25° C. and stirred at 25° C. for 1 hour under N₂ atmosphere. The reaction mixture was quenched with water (5 mL) and extracted with dichloromethane (10 mL×3). The combined organic layers were washed with brine (10 mL×3), dried over Na₂SO₄, filtered, and concentrated under reduced pressure to give a residue. The residue was purified by flash silica gel chromatography (0˜12% ethyl acetate/petroleum ether) to give 4-((1r,4s)-4-(3-bromo-2-(trifluoromethyl)phenoxy)cyclohexyl)butanal (190 mg, 386.53 μmol, 69.45% yield) as a yellow oil.

¹H NMR (400 MHz, DMSO-d₆) δ=9.66 (s, 1H), 7.49-7.40 (m, 1H), 7.39-7.31 (m, 2H), 4.52-4.35 (m, 1H), 2.41 (t, J=6.8 Hz, 2H), 2.04 (d, J=10.4 Hz, 2H), 1.76 (d, J=12.0 Hz, 2H), 1.59-1.46 (m, 2H), 1.42-1.24 (m, 3H), 1.22-1.13 (m, 2H), 1.10-0.97 (m, 2H).

### Step H. Procedure for Preparation of 3-(6-(4-(4-((1r,4s)-4-(3-bromo-2-(trifluoromethyl)phenoxy)cyclohexyl)butyl)piperazin-1-yl)-1-methyl-1H-indazol-3-yl)piperidine-2,6-dione

A solution of 4-((1r,4s)-4-(3-bromo-2-(trifluoromethyl)phenoxy)cyclohexyl)butanal (190 mg, 483.17 μmol, 1.0 equiv.), 3-(1-methyl-6-(piperazin-1-yl)-1H-indazol-3-yl)piperidine-2,6-dione (193.38 mg, 531.48 μmol, 1.1 eq, HCl), and NaOAc (39.64 mg, 483.17 μmol, 1.0 equiv.) in DCM (10 mL) was stirred at room temperature for 12 hours. NaBH(OAc)₃ (512.02 mg, 2.42 mmol, 5 equiv.) was then added. The mixture was stirred at 25° C. for 30 min. The reaction mixture was quenched with water (10 mL) and extracted with dichloromethane (10 mL×3). The combined organic layers were washed with brine (10 mL×3), dried over Na₂SO₄, filtered, and concentrated under reduced pressure to give a residue. The residue was purified by flash silica gel chromatography (0˜20% ethyl acetate/petroleum ether) to give 3-(6-(4-(4-((1r,4s)-4-(3-bromo-2-(trifluoromethyl)phenoxy)cyclohexyl)butyl)piperazin-1-yl)-1-methyl-1H-indazol-3-yl)piperidine-2,6-dione (240 mg, crude) as a white oil.

MS (ESI) m/z: 706.1 [M+H]⁺.

### Step I. Procedure for preparation of tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)butyl)cyclohexyl)oxy)-2-(trifluoromethyl)phenyl)picolinate

A mixture of 3-(6-(4-(4-((1r,4s)-4-(3-bromo-2-(trifluoromethyl)phenoxy)cyclohexyl)butyl)piperazin-1-yl)-1-methyl-1H-indazol-3-yl)piperidine-2,6-dione (240 mg, 340.61 μmol, 1.0 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (250.37 mg, 408.73 μmol, 1.2 equiv.), KF (59.36 mg, 1.02 mmol, 23.94 μL, 3 equiv.), and Ad₂nBuP Pd G₃ (cataCXium® A Pd G₃) (49.61 mg, 68.12 μmol, 0.2 equiv.) in dioxane (4 mL) and H₂O (0.4 mL) was degassed and purged with N₂ three times. The mixture was stirred at 100° C. for 1 hour under N₂ atmosphere. The reaction mixture was filtered and concentrated under reduced pressure to give a residue. The residue was purified by flash silica gel chromatography (0˜20% dichloromethane/methanol) to give tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)butyl)cyclohexyl)oxy)-2-(trifluoromethyl)phenyl)picolinate (300 mg, 221.56 μmol, 65.05% yield) as a yellow solid.

MS (ESI) m/z: 1110.5 [M+H]⁺.

### Step J. Procedure for Preparation of 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)butyl)cyclohexyl)oxy)-2-(trifluoromethyl)phenyl)picolinic acid

To a solution of tert-butyl 6-(8-(benzo[d]thiazol-2-ylcarbamoyl)-3,4-dihydroisoquinolin-2(1H)-yl)-3-(3-(((1S,4R)-4-(4-(4-(3-(2,6-dioxopiperidin-3-yl)-1-methyl-1H-indazol-6-yl)piperazin-1-yl)butyl)cyclohexyl)oxy)-2-(trifluoromethyl)phenyl)picolinate (300 mg, 270.20 μmol, 1.0 equiv.) in DCM (2 mL) was added TFA (3.07 g, 26.92 mmol, 2 mL, 99.65 equiv.). The mixture was stirred at 25° C. for 12 hours. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)butyl)cyclohexyl)oxy)-2-(trifluoromethyl)phenyl)picolinic acid (30.62 mg, 28.20 μmol, 10.44% yield) as a brown solid.

MS (ESI) m/z: 1054.5 [M+H]⁺

¹H NMR (400 MHz, DMSO-d₆) δ=13.27-12.29 (m, 1H), 10.85 (s, 1H), 8.14 (s, 1H), 8.03 (d, J=7.6 Hz, 1H), 7.79 (d, J=8.0 Hz, 1H), 7.62 (d, J=7.6 Hz, 1H), 7.52-7.31 (m, 7H), 7.28-7.22 (m, 1H), 7.02-6.88 (m, 2H), 6.85 (s, 1H), 6.64 (d, J=7.6 Hz, 1H), 5.08-4.87 (m, 2H), 4.48-4.35 (m, 1H), 4.27-4.23 (m, 1H), 3.93 (t, J=5.2 Hz, 2H), 3.89 (s, 3H), 3.24 (s, 6H), 3.02 (t, J=4.8 Hz, 2H), 2.61 (d, J=6.0 Hz, 4H), 2.43 (s, 2H), 2.23-2.13 (m, 2H), 2.11-2.04 (m, 2H), 1.83-1.75 (m, 2H), 1.52-1.44 (m, 2H), 1.38-1.28 (m, 4H), 1.23 (s, 2H), 1.16-0.99 (m, 3H).

### Example 269. Preparation of Compound 308b

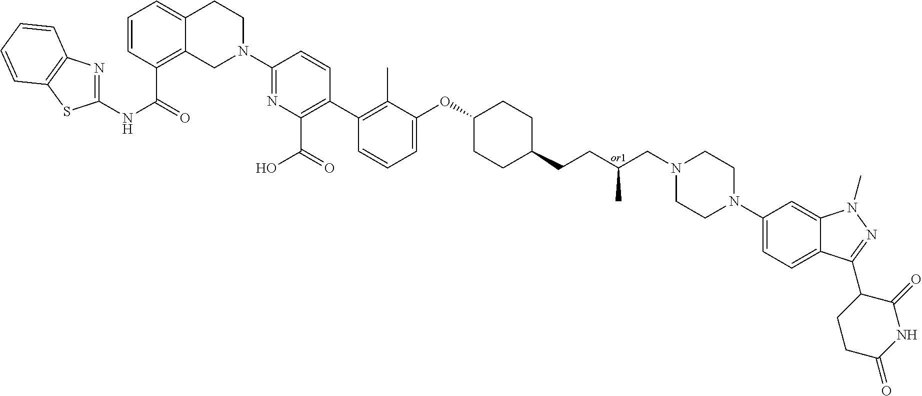 [SMILES: Cc1c(O[C@H]2CC[C@H](CC[C@H](C)CN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-((3S)-4-(4-(3-(2,6-dioxopiperidin-3-yl)-1-methyl-1H-indazol-6-yl)piperazin-1-yl)-3-methylbutyl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1R,4r)-4-((3S*)-4-(4-(3-(2,6-dioxopiperidin-3-yl)-1-methyl-1H-indazol-6-yl)piperazin-1-yl)-3-methylbutyl)cyclohexyl)oxy)-2-methylphenyl)picolinic acid]

### Step A. Procedure for Preparation of (S)-4-((1r,4r)-4-(3-bromo-2-methylphenoxy)cyclohexyl)-2-methylbutanal

A mixture of (S)-4-((1r,4r)-4-(3-bromo-2-methylphenoxy)cyclohexyl)-2-methylbutan-1-ol (120 mg, 337.73 μmol, 1.0 equiv.) and (1,1-diacetoxy-3-oxo-1,2-benziodoxol-1-yl) acetate (286.49 mg, 675.47 μmol, 209.27 μL, 2.0 equiv.) in DCM (5 mL) was degassed and purged with N₂ three times. The mixture was stirred at 25° C. for 3 hours under N₂ atmosphere. The reaction mixture was diluted with water (20 mL) and extracted with EtOAc (25 mL×3). The combined organic layers were washed with brine (15 mL×3), dried over anhydrous sodium sulfate, filtered, and concentrated under reduced pressure to give (S)-4-((1r,4r)-4-(3-bromo-2-methylphenoxy)cyclohexyl)-2-methylbutanal (90 mg, 254.75 μmol, 75.43% yield) as a yellow oil.

### Step B. Procedure for Preparation of 3-(6-(4-((S)-4-((1r,4r)-4-(3-bromo-2-methylphenoxy)cyclohexyl)-2-methylbutyl)piperazin-1-yl)-1-methyl-1H-indazol-3-yl)piperidine-2,6-dione

A mixture of (S)-4-((1r,4r)-4-(3-bromo-2-methylphenoxy)cyclohexyl)-2-methylbutanal (90 mg, 254.75 μmol, 1.0 equiv.), 3-(1-methyl-6-(piperazin-1-yl)-1H-indazol-3-yl)piperidine-2,6-dione (100.08 mg, 305.70 μmol, 1.2 equiv.) in DCM (3 mL) was degassed and purged with N₂ three times, and then the mixture was stirred at 25° C. for 15 hours under N₂ atmosphere. After 15 hours, NaBH(OAc)₃ (161.97 mg, 764.24 μmol, 3.0 equiv.) was added, and the mixture was stirred at 25° C. for 30 minutes under N₂ atmosphere. The reaction mixture was diluted with water (20 mL) and extracted with EtOAc (25 mL×3). The combined organic layers were washed with brine (15 mL)×3, dried over anhydrous sodium sulfate, filtered, and concentrated under reduced pressure to give a residue. The residue was purified by flash silica gel chromatography (0-10% DCM/MeOH) to give 3-(6-(4-((S)-4-((1r,4r)-4-(3-bromo-2-methylphenoxy)cyclohexyl)-2-methylbutyl)piperazin-1-yl)-1-methyl-1H-indazol-3-yl)piperidine-2,6-dione (60 mg, 90.27 μmol, 35.44% yield) as a yellow solid.

MS (ESI) m/z: 666.2 [M+H]⁺

¹H NMR (400 MHz, DMSO-d₆) δ=10.84 (s, 1H), 7.49 (d, J=8.8 Hz, 1H), 7.15-7.12 (m, 1H), 7.07 (t, J=8.0 Hz, 1H), 7.03-7.00 (m, 1H), 6.95-6.88 (m, 1H), 6.84 (s, 1H), 4.31-4.19 (m, 2H), 3.92-3.85 (m, 3H), 3.21 (s, 4H), 2.69-2.65 (m, 2H), 2.63-2.58 (m, 4H), 2.20 (s, 3H), 2.17 (d, J=5.6 Hz, 2H), 2.08-2.02 (m, 2H), 1.83-1.75 (m, 2H), 1.71-1.60 (m, 1H), 1.50-1.38 (m, 2H), 1.37-1.29 (m, 2H), 1.26-1.19 (m, 4H), 1.13-0.98 (m, 4H), 0.88 (d, J=6.4 Hz, 2H)

### Step C. Procedure for Preparation of tert-butyl 6-(8-(benzo[d]thiazol-2-ylcarbamoyl)-3,4-dihydroisoquinolin-2(1H)-yl)-3-(3-(((1r,4r)-4-((3S)-4-(4-(3-(2,6-dioxopiperidin-3-yl)-1-methyl-1H-indazol-6-yl)piperazin-1-yl)-3-methylbutyl)cyclohexyl)oxy)-2-methylphenyl)picolinate

A mixture of 3-(6-(4-((S)-4-((1r,4r)-4-(3-bromo-2-methylphenoxy)cyclohexyl)-2-methylbutyl)piperazin-1-yl)-1-methyl-1H-indazol-3-yl)piperidine-2,6-dione (82.94 mg, 135.40 mol, 1.5 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (60 mg, 90.27 μmol, 1.0 equiv.), KF (1.5 M, 180.54 μL, 3.0 equiv.), and Ad₂nBuP Pd G₃ (cataCXium® A Pd G₃) (6.57 mg, 9.03 μmol, 0.1 equiv.) in dioxane (2 mL) was degassed and purged with N₂ three times. The mixture was stirred at 100° C. for 2 hours under N₂ atmosphere. The reaction mixture was diluted with water (20 mL) and extracted with DCM (25 mL×3). The combined organic layers were washed with brine (15 mL×3), dried over anhydrous sodium sulfate, filtered and concentrated under reduced pressure to give a residue. The residue was purified by prep-TLC (SiO₂, DCM:MeOH=10:1) to give tert-butyl 6-(8-(benzo[d]thiazol-2-ylcarbamoyl)-3,4-dihydroisoquinolin-2(1H)-yl)-3-(3-(((1r,4r)-4-((3S)-4-(4-(3-(2,6-dioxopiperidin-3-yl)-1-methyl-1H-indazol-6-yl)piperazin-1-yl)-3-methylbutyl)cyclohexyl)oxy)-2-methylphenyl)picolinate (90 mg, 79.95 μmol, 88.57% yield) as a brown solid.

MS (ESI) m/z: 1070.8 [M+H]⁺

### Step D. Procedure for Preparation of 6-(8-(benzo[d]thiazol-2-ylcarbamoyl)-3,4-dihydroisoquinolin-2(1H)-yl)-3-(3-(((1r,4r)-4-((3S)-4-(4-(3-(2,6-dioxopiperidin-3-yl)-1-methyl-1H-indazol-6-yl)piperazin-1-yl)-3-methylbutyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1R,4r)-4-((3S*)-4-(4-(3-(2,6-dioxopiperidin-3-yl)-1-methyl-1H-indazol-6-yl)piperazin-1-yl)-3-methylbutyl)cyclohexyl)oxy)-2-methylphenyl)picolinic acid]

A solution of tert-butyl 6-(8-(benzo[d]thiazol-2-ylcarbamoyl)-3,4-dihydroisoquinolin-2(1H)-yl)-3-(3-(((1r,4r)-4-((3S)-4-(4-(3-(2,6-dioxopiperidin-3-yl)-1-methyl-1H-indazol-6-yl)piperazin-1-yl)-3-methylbutyl)cyclohexyl)oxy)-2-methylphenyl)picolinate (90 mg, 84.08 μmol, 1.0 equiv.) in DCM (1 mL) and TFA (0.3 mL) was degassed and purged with N₂ three times. The mixture was stirred at 25° C. for 16 hours under N₂ atmosphere. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1r,4r)-4-((3S)-4-(4-(3-(2,6-dioxopiperidin-3-yl)-1-methyl-1H-indazol-6-yl)piperazin-1-yl)-3-methylbutyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1R,4r)-4-((3S*)-4-(4-(3-(2,6-dioxopiperidin-3-yl)-1-methyl-1H-indazol-6-yl)piperazin-1-yl)-3-methylbutyl)cyclohexyl)oxy)-2-methylphenyl)picolinic acid] (27.48 mg, 26.56 μmol, 31.59% yield) as a white solid.

MS (ESI) m/z: 1014.4 [M+H]⁺

¹H NMR (400 MHz, DMSO-d₆) δ=12.91-12.58 (m, 1H), 10.90-10.78 (m, 1H), 8.14 (d, J=1.2 Hz, 1H), 8.03 (d, J=8.0 Hz, 1H), 7.81-7.75 (m, 1H), 7.62 (d, J=7.6 Hz, 1H), 7.51-7.42 (m, 4H), 7.39-7.33 (m, 2H), 7.10-7.02 (m, 1H), 6.97-6.88 (m, 3H), 6.84 (s, 1H), 6.64-6.57 (m, 1H), 4.97 (s, 2H), 4.28-4.14 (m, 2H), 3.92-3.90 (m, 2H), 3.88 (s, 3H), 3.22-3.20 (m, 4H), 3.03-3.01 (m, 2H), 2.65-2.57 (m, 2H), 2.37-2.24 (m, 2H), 2.20-2.14 (m, 2H), 2.14-2.02 (m, 4H), 1.90-1.85 (m, 3H), 1.81-1.74 (m, 2H), 1.70-1.63 (m, 1H), 1.47-1.41 (m, 1H), 1.35 (dd, J=2.8, 1.6 Hz, 2H), 1.29-1.13 (m, 4H), 1.13-0.96 (m, 4H), 0.91-0.85 (m, 3H)

### Example 270. Preparation of Compound 296a

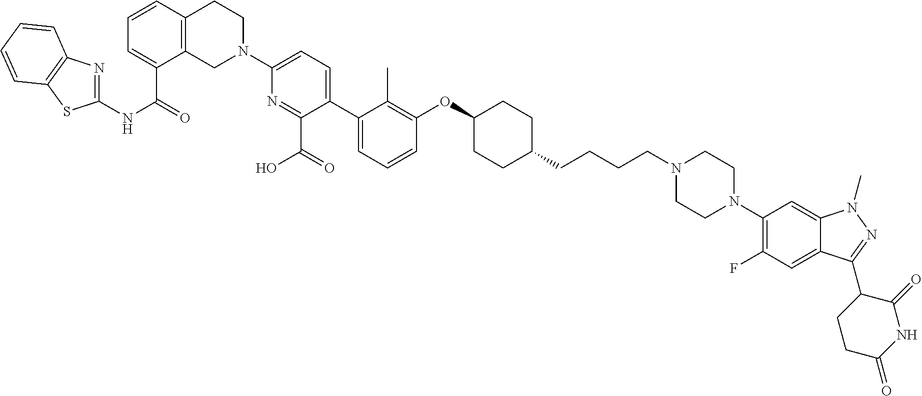 [SMILES: Cc1c(O[C@H]2CC[C@H](CCCCN3CCN(c4cc5c(cc4F)c(C4CCC(=O)NC4=O)nn5C)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-5-fluoro-1-methyl-1H-indazol-6-yl)piperazin-1-yl)butyl)cyclohexyl)oxy)-2-methylphenyl)picolinic acid

### Step A. Procedure for Preparation of 4-((1r,4s)-4-(3-bromo-2-methylphenoxy)cyclohexyl)butanal

To a solution of DMSO (457.88 mg, 5.86 mmol, 457.88 μL, 4 equiv.) in DCM (20 mL) was added oxalyl dichloride (371.91 mg, 2.93 mmol, 256.49 μL, 2 equiv.) in DCM (2 mL) dropwise at −78° C. under N₂ atmosphere. The mixture was stirred at −78° C. for 90 minutes. After which time 4-((1r,4s)-4-(3-bromo-2-methylphenoxy)cyclohexyl)butan-1-ol (500 mg, 1.47 mmol, 1 equiv.) in DCM (2 mL) was added dropwise at −78° C. The reaction mixture was stirred for 30 minutes at −78° C. TEA (889.49 mg, 8.79 mmol, 1.22 mL, 6 equiv.) was then added into the solution. The solution was stirred at 25° C. for 1 hour under N₂ atmosphere. The reaction mixture was quenched by addition of H₂O (5 mL) at 25° C. and extracted with DCM (5 mL×3). The combined organic layers were dried over Na₂SO₄, filtered, and concentrated under reduced pressure to give a residue. The residue was purified by flash silica gel chromatography (0-5% ethyl acetate/petroleum ether) to give 4-((1r,4s)-4-(3-bromo-2-methylphenoxy)cyclohexyl)butanal (400 mg, 1.02 mmol, 69.29% yield) as a yellow oil.

MS (ESI) m/z: 340.1 [M+H]⁺.

¹H NMR (400 MHz, CDCl₃) δ=9.77 (s, 1H), 7.17-7.08 (m, 1H), 6.96 (t, J=8.0 Hz, 1H), 6.83-6.75 (m, 1H), 4.15-4.02 (m, 1H), 2.46-2.40 (m, 2H), 2.29 (s, 3H), 2.17-2.08 (m, 2H), 1.85 (d, J=12.8 Hz, 2H), 1.66 (d, J=7.6, 15.6 Hz, 2H), 1.50-1.38 (m, 3H), 1.28-1.21 (m, 2H), 1.09-0.96 (m, 2H)

### Step B. Procedure for Preparation of tert-butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate

4-((1r,4s)-4-(3-bromo-2-methylphenoxy)cyclohexyl)butanal (400 mg, 1.18 mmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (866.64 mg, 1.41 mmol, 1.2 equiv.), KF (205.49 mg, 3.54 mmol, 82.86 μL, 3 equiv.), and [2-(2-aminophenyl)phenyl]palladium(1+)bis(1-adamantyl)-butyl-phosphane methanesulfonate (171.73 mg, 235.80 μmol, 0.2 equiv.) were taken up into a microwave tube in dioxane (1 mL) and H₂O (0.1 mL). The sealed tube was heated at 100° C. for 1 hour in a microwave reactor. The reaction mixture was concentrated under reduced pressure to give a residue. The residue was purified by flash silica gel chromatography (0˜30% ethyl acetate/petroleum ether) to give tert-butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate (430 mg, 499.30 μmol, 42.35% yield) as a yellow solid.

MS (ESI) m/z: 745.7 [M+H]⁺.

### Step C. Procedure for Preparation of tert-butyl 4-[3-(2,6-dibenzyloxy-3-pyridyl)-5-fluoro-1-methyl-indazol-6-yl]piperazine-1-carboxylate

To a solution of 6-bromo-3-(2,6-dibenzyloxy-3-pyridyl)-5-fluoro-1-methyl-indazole (850 mg, 1.64 mmol, 1 equiv.) and tert-butyl piperazine-1-carboxylate (916.21 mg, 4.92 mmol, 3 equiv.) in toluene (20 mL) was added Pd₂(dba)₃ (150.15 mg, 163.97 μmol, 0.1 equiv.), Cs₂CO₃ (1.60 g, 4.92 mmol, 3 equiv.), and RuPhos (153.03 mg, 327.95 μmol, 0.2 equiv.). The mixture was stirred at 110° C. for 16 hours. The reaction mixture was concentrated under reduced pressure to give a residue. The residue was purified by flash silica gel chromatography (0-10% ethyl acetate/petroleum ether) to give tert-butyl 4-[3-(2,6-dibenzyloxy-3-pyridyl)-5-fluoro-1-methyl-indazol-6-yl]piperazine-1-carboxylate (700 mg, 1.11 mmol, 67.42% yield) as a white solid.

MS (ESI) m/z: 624.5 [M+H]⁺.

### Step D. Procedure for Preparation of tert-butyl 4-[3-(2,6-dioxo-3-piperidyl)-5-fluoro-1-methyl-indazol-6-yl]piperazine-1-carboxylate

To a solution of tert-butyl 4-[3-(2,6-dibenzyloxy-3-pyridyl)-5-fluoro-1-methyl-indazol-6-yl]piperazine-1-carboxylate (600 mg, 961.98 μmol, 1 equiv.) in THF (3 mL) and EtOH (3 mL) was added Pd(OH)₂ (202.64 mg, 144.30 μmol, 10% purity, 0.15 equiv.), Pd/C (200 mg, 144.30 μmol, 10% purity, 0.15 equiv.), and AcOH (173.31 mg, 2.89 mmol, 165.21 μL, 3 equiv.) under a N₂ atmosphere. The suspension was degassed under vacuum and purged with H₂ several times. The mixture was stirred under H₂ (15 psi) at 25° C. for 1 hour. The reaction mixture was filtered and concentrated under reduced pressure to give a residue to give tert-butyl 4-[3-(2,6-dioxo-3-piperidyl)-5-fluoro-1-methyl-indazol-6-yl]piperazine-1-carboxylate (400 mg, 858.39 μmol, 89.23% yield) as a yellow solid.

MS (ESI) m/z: 446.1 [M+H]⁺.

### Step E. Procedure for Preparation of 3-(5-fluoro-1-methyl-6-piperazin-1-yl-indazol-3-yl)piperidine-2,6-dione

A solution of tert-butyl 4-[3-(2,6-dioxo-3-piperidyl)-5-fluoro-1-methyl-indazol-6-yl]piperazine-1-carboxylate (380 mg, 853.00 μmol, 1 equiv.) in HCl/dioxane (2 mL) and DCM (2 mL) was stirred at 25° C. for 5 hours. The reaction mixture was filtered and concentrated under reduced pressure to give a residue to give 3-(5-fluoro-1-methyl-6-piperazin-1-yl-indazol-3-yl)piperidine-2,6-dione (300 mg, 762.66 μmol, 89.41% yield) as a white solid.

MS (ESI) m/z: 345.9 [M+H]⁺.

### Step F. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-5-fluoro-1-methyl-1H-indazol-6-yl)piperazin-1-yl)butyl)cyclohexyl)oxy)-2-methylphenyl)picolinate

A solution of tert-butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate (100 mg, 134.24 μmol, 1 equiv.) and 3-(5-fluoro-1-methyl-6-piperazin-1-yl-indazol-3-yl)piperidine-2,6-dione (46.36 mg, 134.24 μmol, 1 equiv.) in DCM (1.5 mL) was stirred at 25° C. for 15.5 hours. To this solution was added NaBH(OAc)₃ (85.35 mg, 402.72 μmol, 3 equiv.). The resulting mixture was stirred at 25° C. for 0.5 hours. The reaction mixture was concentrated under reduced pressure to give tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-5-fluoro-1-methyl-1H-indazol-6-yl)piperazin-1-yl)butyl)cyclohexyl)oxy)-2-methylphenyl)picolinate (100 mg, 61.16 μmol, 45.56% yield) as a yellow oil.

MS (ESI) m/z: 538.0 [M/2+H]⁺.

### Step G. Procedure for Preparation of 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-5-fluoro-1-methyl-1H-indazol-6-yl)piperazin-1-yl)butyl)cyclohexyl)oxy)-2-methylphenyl)picolinic acid

A solution of tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-5-fluoro-1-methyl-1H-indazol-6-yl)piperazin-1-yl)butyl)cyclohexyl)oxy)-2-methylphenyl)picolinate (90 mg, 83.77 μmol, 1 equiv.) in DCM (1.5 mL) and TFA (1 mL) was stirred at 25° C. for 16 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-5-fluoro-1-methyl-1H-indazol-6-yl)piperazin-1-yl)butyl)cyclohexyl)oxy)-2-methylphenyl)picolinic acid (26.86 mg, 25.88 μmol, 30.89% yield) as a white solid.

MS (ESI) m/z: 1018.5 [M+H]⁺.

¹H NMR (400 MHz, DMSO-d₆) δ=10.85 (s, 1H), 8.16 (s, 1H), 8.03 (d, J=8.0 Hz, 1H), 7.79 (d, J=8.0 Hz, 1H), 7.62 (d, J=7.6 Hz, 1H), 7.51-7.41 (m, 4H), 7.40-7.32 (m, 2H), 7.12-7.02 (m, 2H), 6.93 (dd, J=8.8, 13.6 Hz, 2H), 6.61 (d, J=7.6 Hz, 1H), 4.97 (s, 2H), 4.27 (dd, J=5.2, 9.6 Hz, 1H), 4.24-4.13 (m, 1H), 3.94 (s, 3H), 3.91 (t, J=6.0 Hz, 2H), 3.07 (s, 3H), 3.02 (t, J=5.6 Hz, 2H), 2.68-2.60 (m, 2H), 2.55 (d, J=6.0 Hz, 4H), 2.38-2.31 (m, 3H), 2.13 (dd, J=5.6, 13.6 Hz, 1H), 2.07 (t, J=4.8 Hz, 2H), 1.87 (s, 3H), 1.78 (d, J=10.0 Hz, 2H), 1.58-1.19 (m, 10H), 1.13-0.98 (m, 2H)

### Example 271. Preparation of Compound 306

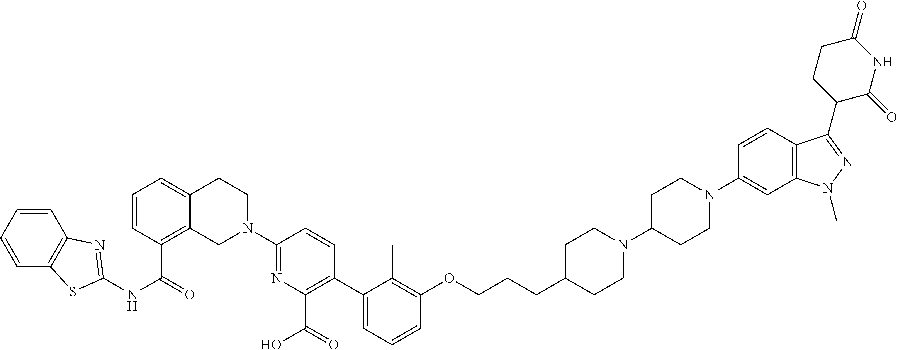 [SMILES: Cc1c(OCCCC2CCN(C3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-(1′-(3-(2,6-dioxopiperidin-3-yl)-1-methyl-1H-indazol-6-yl)-[1,4′-bipiperidin]-4-yl)propoxy)-2-methylphenyl)picolinic acid

### Step A. Preparation of 8-(3-(2,6-bis(benzyloxy)pyridin-3-yl)-1-methyl-1H-indazol-6-yl)-1,4-dioxa-8-azaspiro[4.5]decane

3-(2,6-bis(benzyloxy)pyridin-3-yl)-6-bromo-1-methyl-1H-indazole (500 mg, 1 equiv, 999 μmol) and 1,4-dioxa-8-azaspiro[4.5]decane (172 mg, 153 μL, 1.2 equiv., 1.20 mmol) were suspended in 1,4-dioxane (10 mL). To this mixture (2-dicyclohexylphosphino-2′,6′-diisopropoxy-1,1′-biphenyl)[2-(2′-amino-1,1′-biphenyl)]palladium(II) methanesulfonate (83.6 mg, 0.1 equiv, 99.9 mol), followed by sodium 2-methylpropan-2-olate (288 mg, 1.50 mL, 2.0 molar, 3 equiv, 3.00 mmol), was added dropwise. The mixture was purged with N₂ and heated at 100° C. for 1 hour. The reaction was quenched with water, and concentrated to dryness. The residue was extracted with ethyl acetate and water. The organic layer was separated, washed with brine, dried over Na₂SO₄, filtered, and evaporated to dryness. The residue was then purified by silica gel chromatography (0-100% ethyl acetate in heptane) to afford 8-(3-(2,6-bis(benzyloxy)pyridin-3-yl)-1-methyl-1H-indazol-6-yl)-1,4-dioxa-8-azaspiro[4.5]decane (452 mg, 803 μmol, 80.4%) as an orange oil.

MS (ESI) m/z: 563.4 [M+H]⁺

### Step B. Preparation of 3-(1-methyl-6-(1,4-dioxa-8-azaspiro[4.5]decan-8-yl)-1H-indazol-3-yl)piperidine-2,6-dione

8-(3-(2,6-bis(benzyloxy)pyridin-3-yl)-1-methyl-1H-indazol-6-yl)-1,4-dioxa-8-azaspiro[4.5]decane (450 mg, 1 equiv., 800 μmol) was dissolved in ethyl acetate (10 mL) and DMF (1 mL). To this mixture was added Pd/C (426 mg, 10% Wt, 0.5 equiv., 400 μmol). The resulting mixture was stirred at 50° C. under a H₂ atmosphere overnight. The mixture was filtered through celite and rinsed with ethyl acetate (3 times). The filtrate was then concentrated under reduced pressure to afford 3-(1-methyl-6-(1,4-dioxa-8-azaspiro[4.5]decan-8-yl)-1H-indazol-3-yl)piperidine-2,6-dione (225 mg, 585 μmol, 73.2%) as an off-white solid.

MS (ESI) m/z: 385.1 [M+H]⁺

### Step C. Preparation of 3-(1-methyl-6-(4-oxopiperidin-1-yl)-1H-indazol-3-yl)piperidine-2,6-dione

3-(1-methyl-6-(1,4-dioxa-8-azaspiro[4.5]decan-8-yl)-1H-indazol-3-yl)piperidine-2,6-dione (180 mg, 1 equiv., 468 μmol) was dissolved in water (2 mL) and formic acid (21.6 mg, 17.7 μL, 1 equiv., 468 μmol). The reaction was heated at 60˜80° C. for 8 hours. The reaction was cooled down to room temperature and concentrated to afford 3-(1-methyl-6-(4-oxopiperidin-1-yl)-1H-indazol-3-yl)piperidine-2,6-dione (155 mg, 455 μmol, 97.3%) as a brown solid.

MS (ESI) m/z: 341.1 [M+H]⁺

### Step D. Preparation of 2-methyl-3-(4,4,5,5-tetramethyl-1,3,2-dioxaborolan-2-yl)phenol

3-bromo-2-methylphenol (2 g, 1 equiv, 0.01 mol), 4,4,4′,4′,5,5,5′,5′-octamethyl-2,2′-bi(1,3,2-dioxaborolane) (4 g, 1.5 equiv, 0.02 mol), potassium acetate (3 g, 3 equiv, 0.03 mol), and PdCl₂(dppf)-CH₂Cl₂ adduct (0.4 g, 0.05 equiv, 0.5 mmol) were suspended in 1,4-dioxane (20 mL). The mixture was purged with N₂, stirred, and heated at 85° C. overnight. The mixture was cooled down to room temperature. To this mixture was added ethyl acetate (100 mL). The resulting mixture was filtered and evaporated to dryness. The resulting residue was then purified by silica gel chromatography (0-10% MeOH in DCM) to afford 2-methyl-3-(4,4,5,5-tetramethyl-1,3,2-dioxaborolan-2-yl)phenol (3.2 g, 14 mmol, 100%) as a light yellow solid.

MS (ESI) m/z: 235.3 [M+H]⁺

### Step E. Preparation of tert-butyl 4-(3-(2-methyl-3-(4,4,5,5-tetramethyl-1,3,2-dioxaborolan-2-yl)phenoxy)propyl)piperidine-1-carboxylate

2-methyl-3-(4,4,5,5-tetramethyl-1,3,2-dioxaborolan-2-yl)phenol (300 mg, 1 equiv., 1.28 mmol) was dissolved in acetonitrile (5 mL). To this mixture was added tert-butyl 4-(3-bromopropyl)piperidine-1-carboxylate (471 mg, 1.2 equiv., 1.54 mmol), followed by K₂CO₃ (531 mg, 3 equiv, 3.84 mmol). The mixture was then purged with N₂, stirred, and heated at 70° C. overnight. After completion, the mixture was then cooled down to room temperature, filtered, and rinsed with acetonitrile. The filtrate was then evaporated to dryness. The residue was then purified by silica gel chromatography (0-100% ethyl acetate in heptane) to afford tert-butyl 4-(3-(2-methyl-3-(4,4,5,5-tetramethyl-1,3,2-dioxaborolan-2-yl)phenoxy)propyl)piperidine-1-carboxylate (620 mg, 1.35 mmol, 105%) as a colorless oil, which was carried forward without further purification.

### Step F. Preparation of 4-(3-(2-methyl-3-(4,4,5,5-tetramethyl-1,3,2-dioxaborolan-2-yl)phenoxy)propyl)piperidine, HCl

tert-butyl 4-(3-(2-methyl-3-(4,4,5,5-tetramethyl-1,3,2-dioxaborolan-2-yl)phenoxy)propyl)piperidine-1-carboxylate (620 mg, 1 equiv., 1.35 mmol) was dissolved in HCl in dioxane (4 M, 4 mL). The mixture was then stirred at room temperature for 1 hr. The reaction was concentrated under reduced pressure to afford 4-(3-(2-methyl-3-(4,4,5,5-tetramethyl-1,3,2-dioxaborolan-2-yl)phenoxy)propyl)piperidine, HCl (531 mg, 1.34 mmol, 99.4%) as a white solid.

MS (ESI) m/z: 361.0 [M+H]⁺

### Step G. Preparation of tert-butyl 6-(8-(benzo[d]thiazol-2-ylcarbamoyl)-3,4-dihydroisoquinolin-2(1H)-yl)-3-(2-methyl-3-(3-(piperidin-4-yl)propoxy)phenyl)picolinate

tert-butyl 6-(8-(benzo[d]thiazol-2-ylcarbamoyl)-3,4-dihydroisoquinolin-2(1H)-yl)-3-bromopicolinate (300 mg, 1 equiv., 531 μmol) was suspended in 1,4-dioxane (3 mL) and 1.5 M K₃PO₄ (1 mL). To this mixture was added 4-(3-(2-methyl-3-(4,4,5,5-tetramethyl-1,3,2-dioxaborolan-2-yl)phenoxy)propyl)piperidine, HCl (252 mg, 1.2 equiv., 637 μmol), followed by mesylate[(di(1-adamantyl)-n-butylphosphine)-2-(2′-amino-1,1′-biphenyl)]palladium(II) (38.6 mg, 0.1 equiv., 53.1 μmol). The mixture was then heated at 100° C. for 40 minutes in a microwave reactor. The mixture was extracted with ethyl acetate and water. The organic layer was then separated, washed with brine, dried over Na₂SO₄, filtered, and evaporated to dryness. The crude product was then purified by reverse phase-HPLC to afford tert-butyl 6-(8-(benzo[d]thiazol-2-ylcarbamoyl)-3,4-dihydroisoquinolin-2(1H)-yl)-3-(2-methyl-3-(3-(piperidin-4-yl)propoxy)phenyl)picolinate (103 mg, 143 μmol, 27.0%) as a white solid.

MS (ESI) m/z: 718.5 [M+H]⁺

### Step H. Preparation of tert-butyl 6-(8-(benzo[d]thiazol-2-ylcarbamoyl)-3,4-dihydroisoquinolin-2(1H)-yl)-3-(3-(3-(1′-(3-(2,6-dioxopiperidin-3-yl)-1-methyl-1H-indazol-6-yl)-[1,4′-bipiperidin]-4-yl)propoxy)-2-methylphenyl)picolinate

tert-butyl 6-(8-(benzo[d]thiazol-2-ylcarbamoyl)-3,4-dihydroisoquinolin-2(1H)-yl)-3-(2-methyl-3-(3-(piperidin-4-yl)propoxy)phenyl)picolinate (75 mg, 1 equiv., 0.10 mmol) and 3-(1-methyl-6-(4-oxopiperidin-1-yl)-1H-indazol-3-yl)piperidine-2,6-dione (53 mg, 1.5 equiv, 0.16 mmol) were suspended in DCM (10 mL). To this mixture was added sodium triacetoxyborohydride (66 mg, 3 equiv, 0.31 mmol). The mixture was then purged with N₂ and stirred at room temperature overnight. The reaction mixture was concentrated to dryness and purified by reverse phase-HPLC to afford tert-butyl 6-(8-(benzo[d]thiazol-2-ylcarbamoyl)-3,4-dihydroisoquinolin-2(1H)-yl)-3-(3-(3-(1′-(3-(2,6-dioxopiperidin-3-yl)-1-methyl-1H-indazol-6-yl)-[1,4′-bipiperidin]-4-yl)propoxy)-2-methylphenyl)picolinate (17 mg, 16 μmol, 16%) as a white solid.

MS (ESI) m/z: 521.9 [M+2H]²⁺

### Step I. Preparation of 6-(8-(benzo[d]thiazol-2-ylcarbamoyl)-3,4-dihydroisoquinolin-2(1H)-yl)-3-(3-(3-(1′-(3-(2,6-dioxopiperidin-3-yl)-1-methyl-1H-indazol-6-yl)-[1,4′-bipiperidin]-4-yl)propoxy)-2-methylphenyl)picolinic acid, trifluoroacetic acid

tert-butyl 6-(8-(benzo[d]thiazol-2-ylcarbamoyl)-3,4-dihydroisoquinolin-2(1H)-yl)-3-(3-(3-(1′-(3-(2,6-dioxopiperidin-3-yl)-1-methyl-1H-indazol-6-yl)-[1,4′-bipiperidin]-4-yl)propoxy)-2-methylphenyl)picolinate (17 mg, 1 equiv, 16 μmol) was dissolved in DCM (4 mL). To this mixture was added 2 mL TFA. The mixture was then stirred at room temperature overnight. The mixture was then concentrated to afford 6-(8-(benzo[d]thiazol-2-ylcarbamoyl)-3,4-dihydroisoquinolin-2(1H)-yl)-3-(3-(3-(1′-(3-(2,6-dioxopiperidin-3-yl)-1-methyl-1H-indazol-6-yl)-[1,4′-bipiperidin]-4-yl)propoxy)-2-methylphenyl)picolinic acid, trifluoroacetic acid (18 mg, 16 μmol, 100%) as a white solid.

MS (ESI) m/z: 986.5 [M+H]⁺;

¹H NMR (400 MHz, DMSO) δ 10.78 (s, 1H), 8.90 (s, 1H), 7.96 (d, J=7.9 Hz, 1H), 7.72 (d, J=8.0 Hz, 1H), 7.56 (d, J=7.5 Hz, 1H), 7.48-7.24 (m, 6H), 7.03 (t, J=7.9 Hz, 1H), 6.94-6.78 (m, 4H), 6.57 (d, J=7.5 Hz, 1H), 4.91 (s, 2H), 4.19 (dd, J=9.3, 5.1 Hz, 1H), 3.94-3.83 (m, 5H), 3.83 (s, 3H), 3.43 (d, J=11.5 Hz, 2H), 3.00-2.84 (m, 4H), 2.72 (t, J=12.3 Hz, 2H), 2.60-2.45 (m, 2H), 2.23 (dd, J=9.1, 4.8 Hz, 1H), 2.09 (dt, J=7.6, 5.5 Hz, 1H), 2.04 (s, 1H), 2.01 (s, 1H), 1.92-1.82 (m, 2H), 1.83 (s, 3H), 1.70 (d, J=9.7 Hz, 4H), 1.32 (dd, J=23.5, 10.4 Hz, 4H).

### Example 272. Preparation of Compound 312

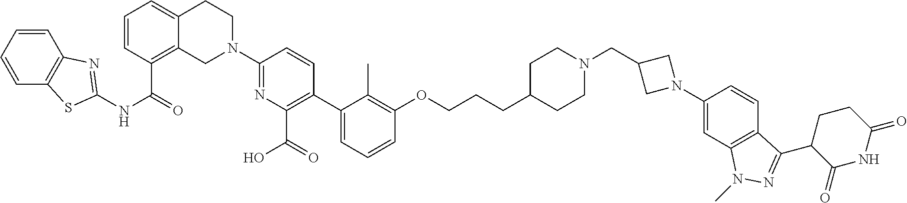 [SMILES: Cc1c(OCCCC2CCN(CC3CN(c4ccc5c(C6CCC(=O)NC6=O)nn(C)c5c4)C3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-(1-((1-(3-(2,6-dioxopiperidin-3-yl)-1-methyl-1H-indazol-6-yl)azetidin-3-yl)methyl)piperidin-4-yl)propoxy)-2-methylphenyl)picolinic acid

### Step A. Preparation of (1-(3-(2,6-bis(benzyloxy)pyridin-3-yl)-1-methyl-1H-indazol-6-yl)azetidin-3-yl)methanol

3-(2,6-bis(benzyloxy)pyridin-3-yl)-6-bromo-1-methyl-1H-indazole (2.0 g, 1 equiv., 4.0 mmol) and azetidin-3-ylmethanol, HCl (0.59 g, 1.2 equiv., 4.8 mmol) were suspended in 1,4-dioxane (20 mL). To this mixture was added (2-dicyclohexylphosphino-2′,6′-diisopropoxy-1,1′-biphenyl)[2-(2′-amino-1,1′-biphenyl)]palladium(II) methanesulfonate (0.33 g, 0.1 equiv., 0.40 mmol). The mixture was purged with N₂. 2-methylpropan-2-olate potassium (0.54 g, 4.8 mL, 1.0 M, 1.2 equiv., 4.8 mmol) was then added dropwise. The mixture was then heated at 40° C. under N₂ and stirred overnight. The mixture was cooled down to room temperature, quenched with water, filtered, and rinsed with ethyl acetate (3×). The filtrate was evaporated to dryness and then purified by silica gel chromatography (0-100% ethyl acetate in heptane) to afford (1-(3-(2,6-bis(benzyloxy)pyridin-3-yl)-1-methyl-1H-indazol-6-yl)azetidin-3-yl)methanol (260 mg, 513 μmol, 13%) as an off-white solid.

MS (ESI) m/z: 507.3 [M+H]⁺

### Step B. Preparation of 3-(6-(3-(hydroxymethyl)azetidin-1-yl)-1-methyl-1H-indazol-3-yl)piperidine-2,6-dione

(1-(3-(2,6-bis(benzyloxy)pyridin-3-yl)-1-methyl-1H-indazol-6-yl)azetidin-3-yl)methanol (260 mg, 1 equiv., 513 μmol) was dissolved in ethyl acetate (5 mL). To the mixture was added Pd(OH)₂ (90.1 mg, 20% Wt, 0.25 equiv., 128 μmol). The mixture was stirred at 50° C. under a H₂ balloon overnight. The mixture was then cooled down to room temperature and filtered through celite. The filtrate was then concentrated and purified by reverse-phase HPLC to afford 3-(6-(3-(hydroxymethyl)azetidin-1-yl)-1-methyl-1H-indazol-3-yl)piperidine-2,6-dione (15 mg, 46 μmol, 8.9%) as a white solid.

MS (ESI) m/z: 329.2 [M+H]⁺

### Step C. Preparation of 1-(3-(2,6-dioxopiperidin-3-yl)-1-methyl-1H-indazol-6-yl)azetidine-3-carbaldehyde

3-(6-(3-(hydroxymethyl)azetidin-1-yl)-1-methyl-1H-indazol-3-yl)piperidine-2,6-dione (30 mg, 1 equiv., 91 μmol) was dissolved in DCM (2 mL). To this mixture was added Dess-Martin periodinane (43 mg, 31 μL, 1.1 equiv., 0.10 mmol). The mixture was stirred at room temperature for ˜2 hrs. The mixture was carried onto the following step without further purification.

### Step D. Preparation of tert-butyl 6-(8-(benzo[d]thiazol-2-ylcarbamoyl)-3,4-dihydroisoquinolin-2(1H)-yl)-3-(3-(3-(1-((1-(3-(2,6-dioxopiperidin-3-yl)-1-methyl-1H-indazol-6-yl)azetidin-3-yl)methyl)piperidin-4-yl)propoxy)-2-methylphenyl)picolinate

tert-butyl 6-(8-(benzo[d]thiazol-2-ylcarbamoyl)-3,4-dihydroisoquinolin-2(1H)-yl)-3-(2-methyl-3-(3-(piperidin-4-yl)propoxy)phenyl)picolinate (15 mg, 1 equiv., 21 μmol) was added to a mixture of 1-(3-(2,6-dioxopiperidin-3-yl)-1-methyl-1H-indazol-6-yl)azetidine-3-carbaldehyde (8 mg, 1.2 equiv., 25 μmol). To this mixture was added sodium triacetoxyborohydride (22 mg, 5 equiv., 0.10 mmol). The mixture was purged with N₂ and stirred at room temperature overnight. The reaction mixture was concentrated to dryness and purified by reverse phase HPLC to afford tert-butyl 6-(8-(benzo[d]thiazol-2-ylcarbamoyl)-3,4-dihydroisoquinolin-2(1H)-yl)-3-(3-(3-(1-((1-(3-(2,6-dioxopiperidin-3-yl)-1-methyl-1H-indazol-6-yl)azetidin-3-yl)methyl)piperidin-4-yl)propoxy)-2-methylphenyl)picolinate (4.7 mg, 4.6 μmol, 22%) as a white solid.

MS (ESI) m/z: 514.9 [M+2H]²⁺

### Step E. Preparation of 6-(8-(benzo[d]thiazol-2-ylcarbamoyl)-3,4-dihydroisoquinolin-2(1H)-yl)-3-(3-(3-(1-((1-(3-(2,6-dioxopiperidin-3-yl)-1-methyl-1H-indazol-6-yl)azetidin-3-yl)methyl)piperidin-4-yl)propoxy)-2-methylphenyl)picolinic acid, trifluoroacetic acid

tert-butyl 6-(8-(benzo[d]thiazol-2-ylcarbamoyl)-3,4-dihydroisoquinolin-2(1H)-yl)-3-(3-(3-(1-((1-(3-(2,6-dioxopiperidin-3-yl)-1-methyl-1H-indazol-6-yl)azetidin-3-yl)methyl)piperidin-4-yl)propoxy)-2-methylphenyl)picolinate (4.7 mg, 1 equiv., 4.6 μmol) was dissolved in DCM (2 mL). To this mixture was added TFA (1 mL). The mixture was then stirred at room temperature for ˜24 hours. The mixture was then concentrated under reduced pressure to afford 6-(8-(benzo[d]thiazol-2-ylcarbamoyl)-3,4-dihydroisoquinolin-2(1H)-yl)-3-(3-(3-(1-((1-(3-(2,6-dioxopiperidin-3-yl)-1-methyl-1H-indazol-6-yl)azetidin-3-yl)methyl)piperidin-4-yl)propoxy)-2-methylphenyl)picolinic acid, trifluoroacetic acid (4.9 mg, 4.5 μmol, 99%) as an off-white solid.

MS (ESI) m/z: 486.9 [M+2H]²⁺;

¹H NMR (400 MHz, DMSO) δ 12.79 (s, 1H), 10.76 (d, J=6.2 Hz, 1H), 7.94 (dd, J=17.4, 8.0 Hz, 2H), 7.72 (d, J=8.0 Hz, 1H), 7.64 (dd, J=7.7, 1.7 Hz, 1H), 7.56 (d, J=7.6 Hz, 1H), 7.46-7.24 (m, 7H), 7.17 (td, J=7.6, 1.7 Hz, 1H), 7.04 (t, J=7.9 Hz, 1H), 6.91 (d, J=9.0 Hz, 1H), 6.82 (d, J=8.2 Hz, 1H), 6.57 (d, J=7.7 Hz, 1H), 6.30 (d, J=9.6 Hz, 1H), 4.91 (s, 2H), 4.22-4.09 (m, 1H), 4.02 (t, J=7.5 Hz, 1H), 3.94-3.83 (m, 4H), 3.78 (s, 2H), 3.57 (t, J=6.7 Hz, 1H), 3.13 (s, 2H), 2.96 (s, 2H), 2.85 (d, J=12.7 Hz, 2H), 2.55 (d, J=6.3 Hz, 1H), 2.47 (s, 1H), 2.08 (dd, J=13.4, 6.0 Hz, 1H), 1.83 (d, J=4.5 Hz, 4H), 1.71 (s, 3H), 1.50 (s, 1H), 1.34 (s, 4H).

### Example 273. Preparation of Compound 318a

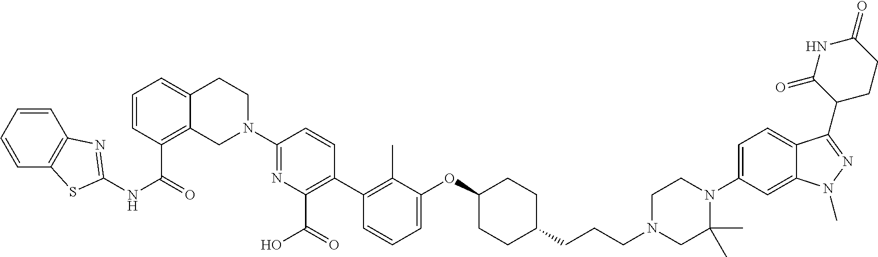 [SMILES: Cc1c(O[C@H]2CC[C@H](CCCN3CCN(c4ccc5c(C6CCC(=O)NC6=O)nn(C)c5c4)C(C)(C)C3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)-3,3-dimethylpiperazin-1-yl)propyl)cyclohexyl)oxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)-3,3-dimethylpiperazin-1-yl)propyl)cyclohexyl)oxy)-2-methylphenyl)picolinate

tert-butyl 6-(8-(benzo[d]thiazol-2-ylcarbamoyl)-3,4-dihydroisoquinolin-2(1H)-yl)-3-(2-methyl-3-(((1r,4r)-4-(3-oxopropyl)cyclohexyl)oxy)phenyl)picolinate (30 mg, 1 equiv., 41 μmol) and 3-(6-(2,2-dimethylpiperazin-1-yl)-1-methyl-1H-indazol-3-yl)piperidine-2,6-dione, HCl (18 mg, 1.1 equiv., 45 μmol) were suspended in DCM (2 mL). To this mixture was added sodium triacetoxyborohydride (26 mg, 18 μL, 3 equiv., 0.12 mmol). The mixture was then stirred at room temperature overnight. The mixture was then extracted with DCM and water. The organic layer was washed with brine, dried over Na₂SO₄, filtered, and evaporated to dryness to afford tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)-3,3-dimethylpiperazin-1-yl)propyl)cyclohexyl)oxy)-2-methylphenyl)picolinate as a light brown solid, which was carried forward without further purification.

MS (ESI) m/z: 535.9 [M+2H]²⁺.

### Step B. Procedure for Preparation of 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)-3,3-dimethylpiperazin-1-yl)propyl)cyclohexyl)oxy)-2-methylphenyl)picolinic acid

tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)-3,3-dimethylpiperazin-1-yl)propyl)cyclohexyl)oxy)-2-methylphenyl)picolinate (44 mg, 1 equiv., 41 μmol) was dissolved in DCM (3 mL). To this mixture was added 1 mL TFA (1.4 g, 0.95 mL, 300 equiv., 12 mmol). The mixture was then stirred at room temperature overnight. The reaction mixture was concentrated, and then purified by reverse phase-HPLC to afford 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)-3,3-dimethylpiperazin-1-yl)propyl)cyclohexyl)oxy)-2-methylphenyl)picolinic acid (21 mg, 21 μmol, 50%) as a white solid.

MS (ESI) m/z: 1014.5 [M+H]⁺;

¹H NMR (400 MHz, DMSO) δ 12.79 (s, 1H), 12.49 (s, 1H), 10.80 (s, 1H), 7.96 (d, J=7.8 Hz, 1H), 7.72 (d, J=8.1 Hz, 1H), 7.56 (d, J=7.5 Hz, 1H), 7.46 (d, J=8.7 Hz, 1H), 7.44-7.35 (m, 3H), 7.29 (dt, J=9.4, 7.3 Hz, 2H), 7.18 (s, 1H), 7.01 (t, J=7.9 Hz, 1H), 6.93-6.80 (m, 3H), 6.55 (d, J=7.5 Hz, 1H), 4.91 (s, 2H), 4.25 (dd, J=9.6, 5.1 Hz, 1H), 4.12 (d, J=10.8 Hz, 1H), 3.86 (d, J=11.1 Hz, 5H), 3.07 (s, 2H), 2.96 (t, J=5.9 Hz, 2H), 2.55 (td, J=10.7, 4.8 Hz, 2H), 2.47 (s, 1H), 2.24 (s, 5H), 2.10 (dd, J=13.3, 5.6 Hz, 1H), 2.03 (s, 2H), 2.00 (s, 1H), 1.80 (s, 3H), 1.74 (d, J=12.5 Hz, 2H), 1.43 (s, 2H), 1.20 (d, J=6.3 Hz, 2H), 1.00 (s, 7H).

### Example 274. Preparation of Compound 322a

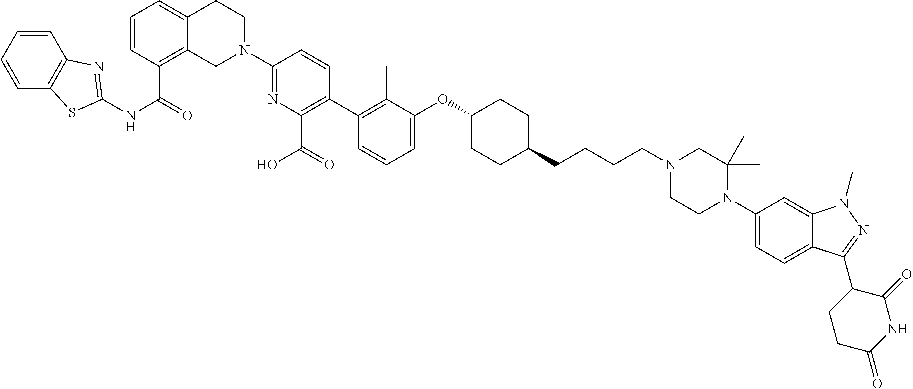 [SMILES: Cc1c(O[C@H]2CC[C@H](CCCCN3CCN(c4ccc5c(C6CCC(=O)NC6=O)nn(C)c5c4)C(C)(C)C3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)-3,3-dimethylpiperazin-1-yl)butyl)cyclohexyl)oxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)-3,3-dimethylpiperazin-1-yl)butyl)cyclohexyl)oxy)-2-methylphenyl)picolinate

tert-butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate (60 mg, 1 equiv., 81 μmol) and 3-(6-(2,2-dimethylpiperazin-1-yl)-1-methyl-1H-indazol-3-yl)piperidine-2,6-dione, HCl (35 mg, 1.1 equiv., 89 μmol) were suspended in DCM (5 mL). To this mixture was added sodium triacetoxyborohydride (51 mg, 3 equiv., 0.24 mmol). The mixture was then stirred at room temperature overnight. The mixture was partitioned between DCM and water. The organic layer was washed with brine, dried over Na₂SO₄, filtered, and evaporated to dryness to afford tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)-3,3-dimethylpiperazin-1-yl)butyl)cyclohexyl)oxy)-2-methylphenyl)picolinate, which was carried forward without further purification.

MS (ESI) m/z: 542.9 [M+2H]²⁺.

### Step B. Procedure for Preparation of 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)-3,3-dimethylpiperazin-1-yl)butyl)cyclohexyl)oxy)-2-methylphenyl)picolinic acid

tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)-3,3-dimethylpiperazin-1-yl)butyl)cyclohexyl)oxy)-2-methylphenyl)picolinate (88 mg, 1 equiv., 81 μmol) was dissolved in DCM (4 mL). To this mixture was added TFA (1 mL). The mixture was then stirred at room temperature overnight. The mixture was then concentrated and purified by reverse phase-HPLC to afford 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)-3,3-dimethylpiperazin-1-yl)butyl)cyclohexyl)oxy)-2-methylphenyl)picolinic acid (41 mg, 40 μmol, 49%) as a white solid.

MS (ESI) m/z: 1028.5 [M+H]⁺;

¹H NMR (400 MHz, DMSO) δ 12.79 (s, 1H), 12.49 (s, 1H), 10.81 (s, 1H), 7.96 (d, J=7.7 Hz, 1H), 7.72 (d, J=8.0 Hz, 1H), 7.59-7.49 (m, 2H), 7.44-7.21 (m, 5H), 7.01 (t, J=7.8 Hz, 1H), 6.93-6.81 (m, 2H), 6.55 (d, J=7.5 Hz, 1H), 4.91 (s, 2H), 4.27 (dd, J=9.6, 5.0 Hz, 1H), 4.14 (s, 1H), 3.88 (d, J=23.4 Hz, 4H), 3.67 (s, 1H), 3.41 (d, J=16.0 Hz, 2H), 3.06 (s, 3H), 2.96 (t, J=6.2 Hz, 2H), 2.89 (s, 1H), 2.56 (d, J=5.9 Hz, 1H), 2.10 (dd, J=13.2, 5.6 Hz, 1H), 2.06-1.98 (m, 2H), 1.80 (s, 2H), 1.73 (d, J=12.7 Hz, 2H), 1.28 (s, 5H), 1.21 (s, 2H), 1.13 (s, 2H), 1.05 (s, 3H), 0.99 (s, 2H).

### Example 275. Preparation of Compound 323a

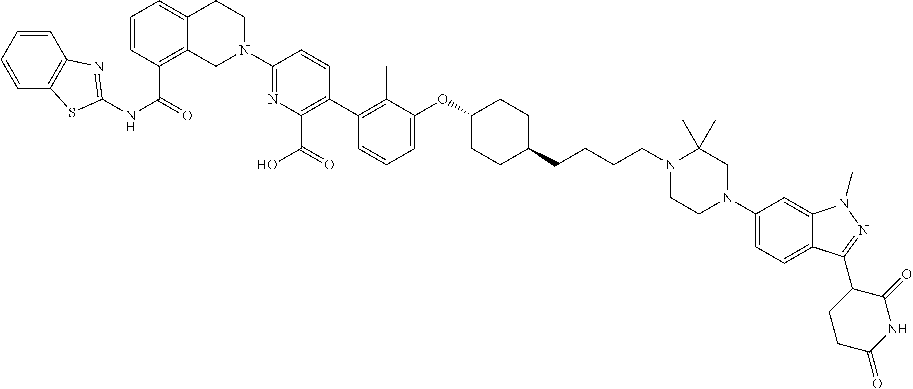 [SMILES: Cc1c(O[C@H]2CC[C@H](CCCCN3CCN(c4ccc5c(C6CCC(=O)NC6=O)nn(C)c5c4)CC3(C)C)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)-2,2-dimethylpiperazin-1-yl)butyl)cyclohexyl)oxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)-2,2-dimethylpiperazin-1-yl)butyl)cyclohexyl)oxy)-2-methylphenyl)picolinate

tert-butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate (60 mg, 1 equiv., 81 μmol) and 3-(6-(3,3-dimethylpiperazin-1-yl)-1-methyl-1H-indazol-3-yl)piperidine-2,6-dione, HCl (31 mg, 1.1 equiv., 89 μmol) were suspended in DCM (5 mL). To this mixture was added sodium triacetoxyborohydride (51 mg, 3 equiv., 0.24 mmol). The mixture was then stirred at room temperature overnight. The mixture was then partitioned between DCM and water. The organic layer was washed with brine, dried over Na₂SO₄, filtered, and evaporated to dryness to afford tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)-2,2-dimethylpiperazin-1-yl)butyl)cyclohexyl)oxy)-2-methylphenyl)picolinate, which was carried forward without further purification.

MS (ESI) m/z: 542.9 [M+2H]²⁺.

### Step B. Procedure for Preparation of 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)-2,2-dimethylpiperazin-1-yl)butyl)cyclohexyl)oxy)-2-methylphenyl)picolinic acid

tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)-2,2-dimethylpiperazin-1-yl)butyl)cyclohexyl)oxy)-2-methylphenyl)picolinate (88 mg, 1 equiv., 81 μmol) was dissolved in DCM (4 mL). To this mixture was added TFA (1 mL). The mixture was stirred at room temperature overnight. The mixture was then concentrated, and purified by reverse phase-HPLC to afford 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)-2,2-dimethylpiperazin-1-yl)butyl)cyclohexyl)oxy)-2-methylphenyl)picolinic acid (34 mg, 33 μmol, 41%) as a white solid.

MS (ESI) m/z: 1028.5 [M+H]⁺;

¹H NMR (400 MHz, DMSO) δ 10.77 (s, 1H), 7.99-7.92 (m, 1H), 7.72 (d, J=8.0 Hz, 1H), 7.55 (d, J=7.7 Hz, 1H), 7.44-7.34 (m, 4H), 7.34-7.23 (m, 2H), 7.00 (t, J=7.9 Hz, 1H), 6.91-6.78 (m, 3H), 6.74 (d, J=1.9 Hz, 1H), 6.54 (d, J=7.4 Hz, 1H), 4.91 (s, 2H), 4.18 (dd, J=9.1, 5.1 Hz, 1H), 3.88-3.79 (m, 2H), 3.82 (s, 3H), 3.11 (d, J=7.7 Hz, 2H), 2.96 (t, J=6.0 Hz, 2H), 2.88 (s, 2H), 2.58 (d, J=6.6 Hz, 3H), 2.58-2.43 (m, 2H), 2.27 (s, 3H), 2.25-2.15 (m, OH), 2.09 (dd, J=13.2, 5.9 Hz, 1H), 2.01 (t, J=5.9 Hz, 2H), 1.80 (s, 3H), 1.72 (d, J=12.6 Hz, 2H), 1.36-1.25 (m, 2H), 1.17 (s, 2H), 0.99 (s, 6H).

### Example 276. Preparation of Compound 324a

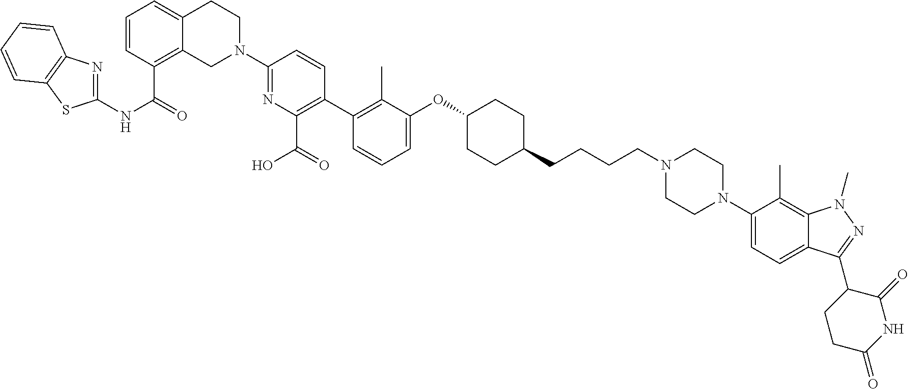 [SMILES: Cc1c(O[C@H]2CC[C@H](CCCCN3CCN(c4ccc5c(C6CCC(=O)NC6=O)nn(C)c5c4C)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1,7-dimethyl-1H-indazol-6-yl)piperazin-1-yl)butyl)cyclohexyl)oxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1,7-dimethyl-1H-indazol-6-yl)piperazin-1-yl)butyl)cyclohexyl)oxy)-2-methylphenyl)picolinate

tert-butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate (60 mg, 1 equiv., 81 μmol) and 3-(1,7-dimethyl-6-(piperazin-1-yl)-1H-indazol-3-yl)piperidine-2,6-dione, HCl (33 mg, 1.1 equiv., 89 μmol) were suspended in DCM (5 mL). To this mixture was added sodium triacetoxyborohydride (51 mg, 3 equiv., 0.24 mmol). The mixture was then stirred at room temperature overnight. The mixture was then extracted DCM and water. The organic layer was washed with brine, dried over Na₂SO₄, filtered, and evaporated to dryness to afford to tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1,7-dimethyl-1H-indazol-6-yl)piperazin-1-yl)butyl)cyclohexyl)oxy)-2-methylphenyl)picolinate, which was carried forward without further purification.

MS (ESI) m/z: 535.9 [M+2H]²⁺.

### Step B. Procedure for Preparation of 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1,7-dimethyl-1H-indazol-6-yl)piperazin-1-yl)butyl)cyclohexyl)oxy)-2-methylphenyl)picolinic acid

tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1,7-dimethyl-1H-indazol-6-yl)piperazin-1-yl)butyl)cyclohexyl)oxy)-2-methylphenyl)picolinate (87 mg, 1 equiv., 81 μmol) was dissolved in DCM (4 mL). To this mixture was added TFA (1 mL). The mixture was stirred at room temperature overnight. The mixture was then concentrated, and purified by reverse phase-HPLC to afford 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1,7-dimethyl-1H-indazol-6-yl)piperazin-1-yl)butyl)cyclohexyl)oxy)-2-methylphenyl)picolinic acid (49 mg, 48 μmol, 60%) as a white solid.

MS (ESI) m/z: 1014.5 [M+H]⁺;

¹H NMR (400 MHz, DMSO) δ 10.78 (s, 1H), 7.99-7.93 (m, 1H), 7.72 (d, J=8.0 Hz, 1H), 7.56 (d, J=7.5 Hz, 1H), 7.44-7.34 (m, 4H), 7.34-7.23 (m, 2H), 7.00 (t, J=7.9 Hz, 1H), 6.93-6.82 (m, 3H), 6.55 (d, J=7.4 Hz, 1H), 4.91 (s, 2H), 4.21 (dd, J=9.6, 5.1 Hz, 1H), 4.11 (s, 3H), 3.85 (t, J=5.9 Hz, 2H), 2.96 (t, J=5.8 Hz, 2H), 2.79 (t, J=4.7 Hz, 4H), 2.61-2.43 (m, 5H), 2.33-2.17 (m, 3H), 2.04 (dt, J=27.1, 5.2 Hz, 3H), 1.80 (s, 3H), 1.72 (d, J=12.4 Hz, 2H), 1.39 (s, 1H), 1.28 (s, 1H), 1.25 (s, 4H), 1.17 (s, 2H), 0.98 (q, J=11.9 Hz, 2H).

### Example 277. Preparation of Compound 325a

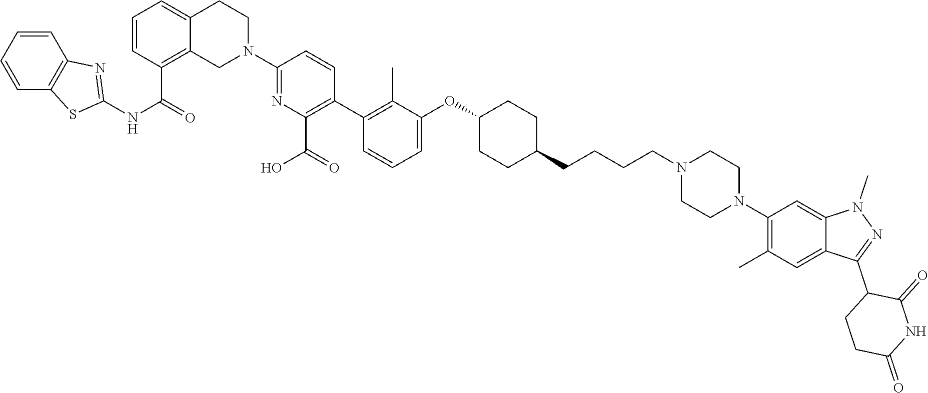 [SMILES: Cc1cc2c(C3CCC(=O)NC3=O)nn(C)c2cc1N1CCN(CCCC[C@H]2CC[C@H](Oc3cccc(-c4ccc(N5CCc6cccc(C(=O)Nc7nc8ccccc8s7)c6C5)nc4C(=O)O)c3C)CC2)CC1]

### 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1, 5-dimethyl-1H-indazol-6-yl)piperazin-1-yl)butyl)cyclohexyl)oxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1,5-dimethyl-1H-indazol-6-yl)piperazin-1-yl)butyl)cyclohexyl)oxy)-2-methylphenyl)picolinate

tert-butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate (60 mg, 1 equiv., 81 μmol) and 3-(1,5-dimethyl-6-(piperazin-1-yl)-1H-indazol-3-yl)piperidine-2,6-dione, HCl (33 mg, 1.1 equiv., 89 μmol) were suspended in DCM (5 mL). To this mixture was added sodium triacetoxyborohydride (51 mg, 3 equiv., 0.24 mmol). The mixture was then stirred at room temperature overnight. The mixture was then partitioned between DCM and water. The organic layer was washed with brine, dried over Na₂SO₄, filtered, and evaporated to dryness to afford to tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1,5-dimethyl-1H-indazol-6-yl)piperazin-1-yl)butyl)cyclohexyl)oxy)-2-methylphenyl)picolinate, which was carried forward without further purification.

MS (ESI) m/z: 535.9 [M+2H]²⁺.

### Step B. Procedure for Preparation of 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1,5-dimethyl-1H-indazol-6-yl)piperazin-1-yl)butyl)cyclohexyl)oxy)-2-methylphenyl)picolinic acid

tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1,5-dimethyl-1H-indazol-6-yl)piperazin-1-yl)butyl)cyclohexyl)oxy)-2-methylphenyl)picolinate (87 mg, 1 equiv., 81 μmol) was dissolved in DCM (4 mL). To this mixture was added TFA (1 mL). The mixture was stirred at room temperature overnight. The mixture was then concentrated, and purified by reverse phase-HPLC to afford 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1,5-dimethyl-1H-indazol-6-yl)piperazin-1-yl)butyl)cyclohexyl)oxy)-2-methylphenyl)picolinic acid (54 mg, 53 μmol, 66%) as a white solid.

MS (ESI) m/z: 1014.5 [M+H]⁺;

¹H NMR (400 MHz, DMSO) δ 10.77 (s, 1H), 7.96 (dd, J=8.0, 1.2 Hz, 1H), 7.72 (d, J=8.0 Hz, 1H), 7.55 (dd, J=7.6, 1.4 Hz, 1H), 7.44-7.33 (m, 4H), 7.33-7.23 (m, 2H), 7.06 (s, 1H), 7.00 (t, J=7.9 Hz, 1H), 6.87 (dd, J=14.8, 8.6 Hz, 2H), 6.55 (d, J=7.4 Hz, 1H), 4.91 (s, 2H), 4.19 (dd, J=9.5, 5.1 Hz, 1H), 4.11 (d, J=10.6 Hz, 1H), 3.85 (s, 3H), 3.88-3.81 (m, 1H), 2.96 (t, J=6.0 Hz, 2H), 2.85 (s, 3H), 2.85 (d, J=9.8 Hz, 1H), 2.61-2.45 (m, 2H), 2.50 (s, 4H), 2.35-2.20 (m, 3H), 2.24 (s, 3H), 2.12-1.98 (m, 1H), 2.02 (s, 2H), 1.80 (s, 3H), 1.72 (d, J=12.4 Hz, 2H), 1.40 (s, 2H), 1.27 (s, 5H), 1.17 (d, J=6.4 Hz, 3H), 1.02 (d, J=11.8 Hz, 1H), 0.96 (d, J=12.1 Hz, 1H).

### Example 278. Preparation of Compound 326a

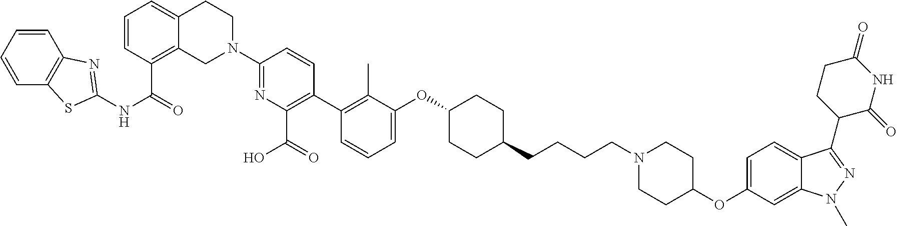 [SMILES: Cc1c(O[C@H]2CC[C@H](CCCCN3CCC(Oc4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(4-(4-((3-(2,6-dioxopiperidin-3-yl)-1-methyl-1H-indazol-6-yl)oxy)piperidin-1-yl)butyl)cyclohexyl)oxy)-2-methylphenyl)picolinic acid

### Step A. Procedure for Preparation of 6-bromo-3-iodo-1-methyl-indazole

To a solution of 6-bromo-3-iodo-1H-indazole (30 g, 92.90 mmol, 1 equiv.) in DMF (200 mL) was added NaH (7.43 g, 185.80 mmol, 60% purity, 2 equiv.) at 0° C. The mixture was stirred for 1 hour. After stirring, Mel (26.37 g, 185.80 mmol, 11.57 mL, 2 equiv.) was added into the solution. The mixture was stirred at 20° C. for 1 hour. After completion, the solution was cooled to 0° C., and then saturated NH₄Cl solution (300 mL) was added dropwise. The solution was extracted with ethyl acetate (500 mL×3). The combined organic layers were washed with brine (500 mL), dried over anhydrous sodium sulfate, filtered, and concentrated under reduced pressure to give a residue. The residue was purified by reverse-phase HPLC to give 6-bromo-3-iodo-1-methyl-indazole (15.3 g, 45.41 mmol, 48.88% yield) as a brown solid. MS (ESI) m/z: 336.8 [M+H]⁺. ¹H NMR (400 MHz, DMSO-d₆) δ=8.02 (d, J=0.6 Hz, 1H), 7.37-7.28 (m, 2H), 4.04 (s, 3H)

### Step B. Procedure for Preparation of 6-bromo-3-(2,6-dibenzyloxy-3-pyridyl)-1-methyl-indazole

A mixture of 6-bromo-3-iodo-1-methyl-indazole (14.8 g, 43.92 mmol, 1 equiv.), 2,6-dibenzyloxy-3-(4,4,5,5-tetramethyl-1,3,2-dioxaborolan-2-yl)pyridine (18.33 g, 43.92 mmol, 1 equiv.), Pd(dppf)Cl₂ (3.21 g, 4.39 mmol, 0.1 equiv.), Cs₂CO₃ (42.93 g, 131.77 mmol, 3 equiv.), and H₂O (10 mL) in THE (100 mL) was degassed and purged with N₂ three times. The mixture was stirred at 60° C. for 16 hours under N₂ atmosphere. After completion, the reaction mixture was poured into water (200 mL) and extracted with ethyl acetate (150 mL×3). The combined organic layers were washed with brine (200 mL), dried over anhydrous sodium sulfate, filtered, and concentrated under reduced pressure to give a residue. The residue was purified by column chromatography (petroleum ether/ethyl acetate=100/1 to 20/1) to give 6-bromo-3-(2,6-dibenzyloxy-3-pyridyl)-1-methyl-indazole (14 g, 27.98 mmol, 63.70% yield) as a yellow solid. ¹H NMR (400 MHz, DMSO-d₆) δ=7.94 (d, J=1.2 Hz, 1H), 7.91 (d, J=8.0 Hz, 1H), 7.61 (d, J=8.4 Hz, 1H), 7.51-7.44 (m, 2H), 7.42-7.23 (m, 8H), 7.10 (dd, J=1.6, 8.8 Hz, 1H), 6.59 (d, J=8.0 Hz, 1H), 5.44 (d, J=8.4 Hz, 4H), 4.04 (s, 3H)

### Step C. Procedure for Preparation of 3-(2,6-dibenzyloxy-3-pyridyl)-1-methyl-indazol-6-ol

A mixture of 6-bromo-3-(2,6-dibenzyloxy-3-pyridyl)-1-methyl-indazole (13 g, 25.98 mmol, 1 equiv.), H₂O (936.07 mg, 51.96 mmol, 936.07 μL, 2 equiv.), NaO^{t}Bu (7.49 g, 77.94 mmol, 3 equiv.), [2-(2-aminophenyl)phenyl]-methylsulfonyloxy-palladium; ditert-butyl-[3,6-dimethoxy-2-(2,4,6-triisopropylphenyl)phenyl]phosphane (22.20 g, 25.98 mmol, 1 equiv.) in THE (100 mL) was degassed and purged with N₂ three times. The mixture was stirred at 80° C. for 16 hours under N₂ atmosphere. The reaction mixture was concentrated under reduced pressure to give a residue. The residue was purified by column chromatography (petroleum ether/ethyl acetate=20/1 to 1/1) to give 3-(2,6-dibenzyloxy-3-pyridyl)-1-methyl-indazol-6-ol (11 g, 16.77 mmol, 64.55% yield) as a colorless oil. ¹H NMR (400 MHz, DMSO-d₆) δ=9.65 (s, 1H), 7.87 (d, J=8.0 Hz, 1H), 7.50-7.44 (m, 3H), 7.43-7.27 (m, 8H), 6.74 (d, J=2.0 Hz, 1H), 6.59-6.52 (m, 2H), 5.43 (d, J=9.2 Hz, 4H), 3.91 (s, 3H)

### Step D. Procedure for Preparation of tert-butyl 4-[3-(2,6-dibenzyloxy-3-pyridyl)-1-methyl-indazol-6-yl]oxypiperidine-1-carboxylate

A mixture of 3-(2,6-dibenzyloxy-3-pyridyl)-1-methyl-indazol-6-ol (5.5 g, 12.57 mmol, 1 equiv.), tert-butyl 4-hydroxypiperidine-1-carboxylate (5.06 g, 25.14 mmol, 2 equiv.) and 2-(tributyl-λ⁵-phosphanylidene)acetonitrile (3.34 g, 13.83 mmol, 1.1 equiv.) in toluene (100 mL) was degassed and purged with N₂ three times. The mixture was stirred at 110° C. for 16 hours under N₂ atmosphere. The reaction mixture was concentrated under reduced pressure to give a residue. The residue was purified by column chromatography (petroleum ether/ethyl acetate=20/1 to 2/1) to give tert-butyl 4-[3-(2,6-dibenzyloxy-3-pyridyl)-1-methyl-indazol-6-yl]oxypiperidine-1-carboxylate (16 g, crude) as a yellow oil). ¹H NMR (400 MHz, DMSO-d₆) δ=7.89 (d, J=8.0 Hz, 1H), 7.54 (d, J=8.8 Hz, 1H), 7.50-7.45 (m, 2H), 7.43-7.24 (m, 8H), 7.13 (d, J=1.6 Hz, 1H), 6.66 (dd, J=2.0, 8.8 Hz, 1H), 6.58 (d, J=8.0 Hz, 1H), 5.48-5.40 (m, 4H), 4.70-4.64 (m, 1H), 4.00 (s, 3H), 3.30-3.15 (m, 2H), 2.95 (s, 3H), 2.03-1.92 (m, 2H), 1.60-1.53 (m, 2H), 1.39 (s, 9H)

### Step E. Procedure for Preparation of tert-butyl 4-[3-(2,6-dioxo-3-piperidyl)-1-methyl-indazol-6-yl]oxypiperidine-1-carboxylate

A mixture of tert-butyl 4-[3-(2,6-dibenzyloxy-3-pyridyl)-1-methyl-indazol-6-yl]oxypiperidine-1-carboxylate (16 g, 25.78 mmol, 1 equiv.), Pd/C (3 g, 25.78 mmol, 10% purity, 1 equiv.), AcOH (1.55 g, 25.78 mmol, 1.48 mL, 1 equiv.), Pd(OH)₂ (3 g, 2.14 mmol, 10% purity, 0.0829 equiv.), and EtOH (100 mL) in THE (100 mL) was degassed and purged with H₂ (50 Psi) three times. The mixture was stirred at 80° C. for 16 hours under H₂ (50 Psi) atmosphere. The reaction mixture was filtered and concentrated under reduced pressure to give a residue. The residue was purified by reversed-phase HPLC (with 0.1% TFA) to give tert-butyl 4-[3-(2,6-dioxo-3-piperidyl)-1-methyl-indazol-6-yl]oxypiperidine-1-carboxylate (3.8 g, 8.59 mmol, 33.32% yield) as a colorless oil. ¹H NMR (400 MHz, DMSO-d₆) δ=10.87 (s, 1H), 7.57 (d, J=8.8 Hz, 1H), 7.13 (d, J=1.6 Hz, 1H), 6.77 (dd, J=1.6, 8.8 Hz, 1H), 4.74-4.65 (m, 1H), 4.30 (dd, J=5.2, 9.6 Hz, 1H), 3.93 (s, 3H), 3.74-3.63 (m, 2H), 3.29-3.16 (m, 2H), 2.71-2.56 (m, 2H), 2.39-2.27 (m, 1H), 2.21-2.12 (m, 1H), 2.02-1.92 (m, 2H), 1.64-1.52 (m, 2H), 1.42 (s, 9H)

### Step F. Procedure for Preparation of 3-[1-methyl-6-(4-piperidyloxy)indazol-3-yl]piperidine-2,6-dione

To a solution of tert-butyl 4-[3-(2,6-dioxo-3-piperidyl)-1-methyl-indazol-6-yl]oxypiperidine-1-carboxylate (3.8 g, 8.59 mmol, 1 equiv.) in HCl/dioxane (30 mL) DCM (10 mL) was stirred at 25° C. for 1 hour. The reaction mixture was filtered to give a filter cake. The filter cake was triturated with MeCN at 25° C. for 10 minutes to give 3-[1-methyl-6-(4-piperidyloxy)indazol-3-yl]piperidine-2,6-dione (2.20 g, 5.82 mmol, 67.74% yield) as a white solid. MS (ESI) m/z: 343.2 [M+H]⁺. 1H NMR (400 MHz, DMSO-d₆) δ=10.87 (s, 1H), 9.39 (s, 2H), 7.59 (d, J=8.8 Hz, 1H), 7.21 (d, J=1.6 Hz, 1H), 6.79 (dd, J=2.0, 8.8 Hz, 1H), 6.02 (s, 4H), 4.88-4.73 (m, 1H), 4.31 (dd, J=5.2, 9.6 Hz, 1H), 3.97-3.91 (m, 3H), 3.22 (s, 2H), 3.12-3.01 (m, 2H), 2.72-2.54 (m, 2H), 2.35-2.26 (m, 1H), 2.22-2.11 (m, 3H), 1.97-1.87 (m, 2H).

### Step G. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(4-(4-((3-(2,6-dioxopiperidin-3-yl)-1-methyl-1H-indazol-6-yl)oxy)piperidin-1-yl)butyl)cyclohexyl)oxy)-2-methylphenyl)picolinate

tert-butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate (60 mg, 1 equiv., 81 μmol) and 3-[1-methyl-6-(4-piperidyloxy)indazol-3-yl]piperidine-2,6-dione, HCl (34 mg, 1.1 equiv., 89 mol) were suspended in DCM (5 mL). To this mixture was added sodium triacetoxyborohydride (51 mg, 3 equiv., 0.24 mmol). The mixture was then stirred at room temperature overnight. The mixture was then partitioned between DCM and water. The organic layer was washed with brine, dried over Na₂SO₄, filtered, and evaporated to dryness to afford to tert-butyl 6-(8-(benzo[d]thiazol-2-ylcarbamoyl)-3,4-dihydroisoquinolin-2(1H)-yl)-3-(3-(((1s,4r)-4-(4-(4-((3-(2,6-dioxopiperidin-3-yl)-1-methyl-1H-indazol-6-yl)oxy)piperidin-1-yl)butyl)cyclohexyl)oxy)-2-methylphenyl)picolinate, which was carried forward without further purification.

MS (ESI) m/z: 536.4 [M+2H]²⁺.

### Step H. Procedure for Preparation of 6-(8-(benzo[d]thiazol-2-ylcarbamoyl)-3,4-dihydroisoquinolin-2(1H)-yl)-3-(3-(((1s,4r)-4-(4-(4-((3-(2,6-dioxopiperidin-3-yl)-1-methyl-1H-indazol-6-yl)oxy)piperidin-1-yl)butyl)cyclohexyl)oxy)-2-methylphenyl)picolinic acid

tert-butyl 6-(8-(benzo[d]thiazol-2-ylcarbamoyl)-3,4-dihydroisoquinolin-2(1H)-yl)-3-(3-(((1s,4r)-4-(4-(4-((3-(2,6-dioxopiperidin-3-yl)-1-methyl-1H-indazol-6-yl)oxy)piperidin-1-yl)butyl)cyclohexyl)oxy)-2-methylphenyl)picolinate (87 mg, 1 equiv., 81 μmol) was dissolved in DCM (4 mL). To the mixture was added TFA (1 mL). The mixture was stirred at room temperature overnight. The mixture was then concentrated, and purified by reverse phase-HPLC to afford 6-(8-(benzo[d]thiazol-2-ylcarbamoyl)-3,4-dihydroisoquinolin-2(1H)-yl)-3-(3-(((1s,4r)-4-(4-(4-((3-(2,6-dioxopiperidin-3-yl)-1-methyl-1H-indazol-6-yl)oxy)piperidin-1-yl)butyl)cyclohexyl)oxy)-2-methylphenyl)picolinic acid (46 mg, 45 μmol, 56%) as a white solid.

MS (ESI) m/z: 1015.5 [M+H]⁺;

¹H NMR (400 MHz, DMSO) δ 10.78 (s, 1H), 7.96 (d, J=8.1 Hz, 1H), 7.72 (d, J=8.0 Hz, 1H), 7.56 (d, J=7.4 Hz, 1H), 7.48 (d, J=8.8 Hz, 1H), 7.44-7.34 (m, 3H), 7.34-7.23 (m, 2H), 7.04-6.96 (m, 2H), 6.86 (dd, J=13.8, 8.6 Hz, 2H), 6.67 (dd, J=8.8, 2.1 Hz, 1H), 6.55 (d, J=7.5 Hz, 1H), 4.91 (s, 2H), 4.45 (s, 1H), 4.22 (dd, J=9.4, 5.1 Hz, 1H), 4.12 (s, 1H), 3.85 (s, 4H), 3.88-3.81 (m, 1H), 2.96 (t, J=6.0 Hz, 2H), 2.66 (s, 2H), 2.60-2.43 (m, 2H), 2.28 (d, J=7.7 Hz, 1H), 2.22 (s, 1H), 2.09 (dd, J=13.2, 5.8 Hz, 1H), 2.00 (q, J=4.4 Hz, 2H), 1.80 (s, 3H), 1.71 (d, J=12.4 Hz, 2H), 1.60 (d, J=10.1 Hz, 3H), 1.35 (d, J=7.1 Hz, 2H), 1.22 (s, 8H), 1.16 (s, 1H), 0.97 (q, J=12.3 Hz, 2H).

### Example 279. Preparation of Compound 327a

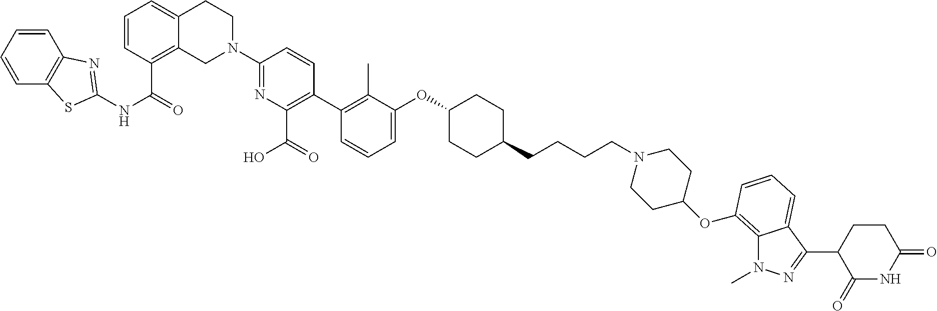 [SMILES: Cc1c(O[C@H]2CC[C@H](CCCCN3CCC(Oc4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(4-(4-((3-(2,6-dioxopiperidin-3-yl)-1-methyl-1H-indazol-7-yl)oxy)piperidin-1-yl)butyl)cyclohexyl)oxy)-2-methylphenyl)picolinic acid

### Step A. Procedure for Preparation of 7-bromo-3-iodo-1-methyl-indazole

To a solution of 7-bromo-3-iodo-1H-indazole (20 g, 61.93 mmol, 1.0 equiv.) in THE (50 mL) was added t-BuOK (13.90 g, 123.87 mmol, 2.0 equiv.) at 0° C. for 1 hour. Then CH₃I (26.37 g, 185.80 mmol, 11.57 mL, 3.0 equiv.) was added to the mixture at 0° C. The mixture was stirred at 20° C. for 2 hours under N₂ atmosphere. The reaction mixture was filtered and concentrated under reduced pressure to give a residue. The residue was purified by reverse-phase HPLC to give 7-bromo-3-iodo-1-methyl-indazole (15 g, 42.29 mmol, 54.63% yield) as a yellow solid. MS (ESI) m/z: 336.6 [M+H]⁺

### Step B. Procedure for Preparation of 7-bromo-3-(2,6-dibenzyloxy-3-pyridyl)-1-methyl-indazole

A mixture of 2,6-dibenzyloxy-3-(4,4,5,5-tetramethyl-1,3,2-dioxaborolan-2-yl)pyridine (17.34 g, 41.55 mmol, 1.0 equiv.), 7-bromo-3-iodo-1-methyl-indazole (14 g, 41.55 mmol, 1.0 equiv.), [1,1′-bis(diphenylphosphino)ferrocene]dichloropalladium(II) (6.79 g, 8.31 mmol, 0.2 equiv.), and Cs₂CO₃ (40.61 g, 124.65 mmol, 3.0 equiv.) in THE (200 mL) and H₂O (40 mL) was degassed and purged with N₂ three times. The mixture was stirred at 85° C. for 1 hour under N₂ atmosphere. The reaction mixture was concentrated under reduced pressure to give a residue. The residue was purified by column chromatography (petroleum ether/ethyl acetate=I/O to 10/1) to give 7-bromo-3-(2,6-dibenzyloxy-3-pyridyl)-1-methyl-indazole (15.1 g, 28.06 mmol, 63.04% yield) as a white solid. MS (ESI) m/z: 502.3 [M+H]⁺

### Step C. Procedure for Preparation of 3-(2,6-dibenzyloxy-3-pyridyl)-1-methyl-indazol-7-ol

A solution of 7-bromo-3-(2,6-dibenzyloxy-3-pyridyl)-1-methyl-indazole (15 g, 29.98 mmol, 1.0 equiv.) in THE (300 mL) was added NaOt-Bu (8.64 g, 89.93 mmol, 3.0 equiv.), [(2-di-tert-butylphosphino-3,6-dimethoxy-2′,4′,6′-triisopropyl-1,1′-biphenyl)-2-(2′-amino-1,1′-biphenyl)]palladium(II) methanesulfonate (2.56 g, 3.00 mmol, 0.1 equiv.), and H₂O (1.08 g, 59.95 mmol, 1.08 mL, 2.0 equiv.) was stirred at 60° C. for 12 hours. The reaction mixture was concentrated under reduced pressure to give a residue. The residue was purified by column chromatography (petroleum ether/ethyl acetate=1/0 to 4/1) to give 3-(2,6-dibenzyloxy-3-pyridyl)-1-methyl-indazol-7-ol (5.6 g, 11.52 mmol, 38.43% yield) as a brown oil. MS (ESI) m/z: 438.6 [M+H]⁺

### Step D. Procedure for Preparation of tert-butyl 4-((3-(2,6-bis(benzyloxy)pyridin-3-yl)-1-methyl-1H-indazol-7-yl)oxy)piperidine-1-carboxylate

A solution of 3-(2,6-dibenzyloxy-3-pyridyl)-1-methyl-indazol-7-ol (2.8 g, 6.40 mmol, 1.0 equiv.) in toluene (100 mL) was added 2-(tributyl-1-phosphanylidene) acetonitrile (3.09 g, 12.80 mmol, 2.0 equiv.) and tert-butyl 4-hydroxypiperidine-1-carboxylate (1.55 g, 7.68 mmol, 1.2 equiv.). The reaction mixture was stirred at 110° C. for 16 hours. The reaction mixture was concentrated under reduced pressure to give a residue. The residue was purified by column chromatography (petroleum ether/ethyl acetate=20/1 to 5/1) to give tert-butyl 4-((3-(2,6-bis(benzyloxy)pyridin-3-yl)-1-methyl-1H-indazol-7-yl)oxy)piperidine-1-carboxylate (4.5 g, 6.52 mmol, 50.97% yield) as a yellow solid. MS (ESI) m/z: 621.6 [M+H]⁺

### Step E. Procedure for Preparation of tert-butyl 4-((3-(2,6-dioxopiperidin-3-yl)-1-methyl-1H-indazol-7-yl)oxy)piperidine-1-carboxylate

To a solution of tert-butyl 4-((3-(2,6-bis(benzyloxy)pyridin-3-yl)-1-methyl-1H-indazol-7-yl)oxy)piperidine-1-carboxylate (4.5 g, 7.25 mmol, 1.0 equiv.) in EtOH (50 mL) and THF (50 mL) were added AcOH (870.66 mg, 14.50 mmol, 829.20 μL, 2.0 equiv.), Pd(OH)₂ (3 g, 4.27 mmol, 20% purity, 0.589 equiv.), and Pd/C (3 g, 3.62 mmol, 10% purity, 0.5 equiv.) under N₂ atmosphere. The suspension was degassed and purged with H₂ three times. The mixture was stirred under H₂ (50 Psi) at 50° C. for 12 hours. The reaction mixture was concentrated under reduced pressure to give a residue. The crude product was purified by reverse-phase HPLC to give tert-butyl 4-((3-(2,6-dioxopiperidin-3-yl)-1-methyl-1H-indazol-7-yl)oxy)piperidine-1-carboxylate (2.58 g, 5.71 mmol, 78.81% yield) as a white solid. MS (ESI) m/z: 443.0 [M+H]⁺

### Step F. Procedure for Preparation of 3-(1-methyl-7-(piperidin-4-yloxy)-1H-indazol-3-yl)piperidine-2,6-dione, HCl

To a solution of tert-butyl 4-((3-(2,6-dioxopiperidin-3-yl)-1-methyl-1H-indazol-7-yl)oxy)piperidine-1-carboxylate (2.58 g, 5.82 mmol, 1.0 eq) in DCM (20 mL) was added HCl/dioxane (4 M, 1.46 mL, 1.0 equiv.). The mixture was stirred at 25° C. for 2 hours. The reaction mixture was filtered and concentrated under reduced pressure to give 3-(1-methyl-7-(piperidin-4-yloxy)-1H-indazol-3-yl)piperidine-2,6-dione, HCl (1.01 g, 2.49 mmol, 42.74% yield) as a yellow solid which was used into next steps without further purification. MS (ESI) m/z: 343.2 [M+H]⁺.

¹H NMR (400 MHz, DMSO-d₆) δ=10.87 (s, 1H), 9.54 (s, 1H), 9.32 (s, 1H), 7.24 (d, J=7.6 Hz, 1H), 7.02-6.90 (m, 2H), 4.92-4.83 (m, 1H), 4.33 (dd, J=5.2, 9.8 Hz, 1H), 4.19 (s, 3H), 3.20 (s, 2H), 3.12 (s, 2H), 2.72-2.55 (m, 2H), 2.34-2.13 (m, 4H), 2.07-1.98 (m, 2H)

### Step G. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(4-(4-((3-(2,6-dioxopiperidin-3-yl)-1-methyl-1H-indazol-7-yl)oxy)piperidin-1-yl)butyl)cyclohexyl)oxy)-2-methylphenyl)picolinate

tert-butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate (60 mg, 1 equiv., 81 μmol) and 3-(1-methyl-7-(piperidin-4-yloxy)-1H-indazol-3-yl)piperidine-2,6-dione, HCl (34 mg, 1.1 equiv., 89 mol) were suspended in DCM (5 mL). To this mixture was added sodium triacetoxyborohydride (51 mg, 3 equiv., 0.24 mmol). The mixture was then stirred at room temperature overnight. The mixture was then partitioned between DCM and water. The organic layer was washed with brine, dried over Na₂SO₄, filtered, and evaporated to dryness to afford to tert-butyl 6-(8-(benzo[d]thiazol-2-ylcarbamoyl)-3,4-dihydroisoquinolin-2(1H)-yl)-3-(3-(((1s,4r)-4-(4-(4-((3-(2,6-dioxopiperidin-3-yl)-1-methyl-1H-indazol-7-yl)oxy)piperidin-1-yl)butyl)cyclohexyl)oxy)-2-methylphenyl)picolinate, which was carried forward without further purification.

MS (ESI) m/z: 536.3 [M+2H]²⁺.

### Step H. Procedure for Preparation of 6-(8-(benzo[d]thiazol-2-ylcarbamoyl)-3,4-dihydroisoquinolin-2(1H)-yl)-3-(3-(((1s,4r)-4-(4-(4-((3-(2,6-dioxopiperidin-3-yl)-1-methyl-1H-indazol-7-yl)oxy)piperidin-1-yl)butyl)cyclohexyl)oxy)-2-methylphenyl)picolinic acid

tert-butyl 6-(8-(benzo[d]thiazol-2-ylcarbamoyl)-3,4-dihydroisoquinolin-2(1H)-yl)-3-(3-(((1s,4r)-4-(4-(4-((3-(2,6-dioxopiperidin-3-yl)-1-methyl-1H-indazol-7-yl)oxy)piperidin-1-yl)butyl)cyclohexyl)oxy)-2-methylphenyl)picolinate (87 mg, 1 equiv., 81 μmol) was dissolved in DCM (4 mL). To this mixture was added TFA (1 mL). The mixture was stirred at room temperature overnight. The mixture was then concentrated, and purified by reverse phase-HPLC to afford 6-(8-(benzo[d]thiazol-2-ylcarbamoyl)-3,4-dihydroisoquinolin-2(1H)-yl)-3-(3-(((1s,4r)-4-(4-(4-((3-(2,6-dioxopiperidin-3-yl)-1-methyl-1H-indazol-7-yl)oxy)piperidin-1-yl)butyl)cyclohexyl)oxy)-2-methylphenyl)picolinic acid (16 mg, 16 μmol, 19%) as a white solid.

MS (ESI) m/z: 1015.4 [M+H]⁺;

¹H NMR (400 MHz, DMSO_{d6}) δ 10.80 (s, 1H), 7.96 (d, J=8.0 Hz, 1H), 7.72 (d, J=8.1 Hz, 1H), 7.55 (d, J=7.5 Hz, 1H), 7.44-7.34 (m, 3H), 7.34-7.23 (m, 2H), 7.14 (d, J=8.0 Hz, 1H), 7.00 (t, J=7.9 Hz, 1H), 6.95-6.78 (m, 4H), 6.54 (d, J=7.5 Hz, 1H), 4.91 (s, 2H), 4.59 (s, 1H), 4.24 (dd, J=9.8, 5.1 Hz, 1H), 4.13 (s, 3H), 3.85 (t, J=5.9 Hz, 2H), 2.96 (t, J=6.0 Hz, 2H), 2.60-2.45 (m, 1H), 2.13-1.98 (m, 1H), 1.99 (s, 3H), 1.80 (s, 3H), 1.71 (d, J=12.8 Hz, 2H), 1.39 (s, 2H), 1.23 (s, 7H), 1.16 (s, 1H), 0.98 (q, J=11.8 Hz, 2H).

### Example 280. Preparation of Compound 301a

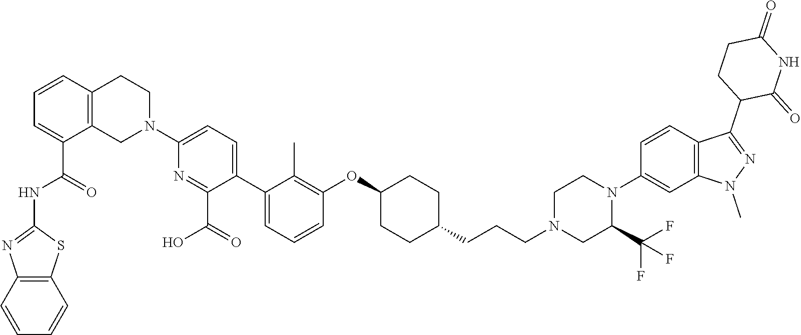 [SMILES: Cc1c(O[C@H]2CC[C@H](CCCN3CCN(c4ccc5c(C6CCC(=O)NC6=O)nn(C)c5c4)[C@@H](C(F)(F)F)C3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(3-((3R)-4-(3-(2,6-dioxopiperidin-3-yl)-1-methyl-1H-indazol-6-yl)-3-(trifluoromethyl)piperazin-1-yl)propyl)cyclohexyl)oxy)-2-methylphenyl)picolinic acid

### Step A. tert-butyl 6-(8-(benzo[d]thiazol-2-ylcarbamoyl)-3,4-dihydroisoquinolin-2(1H)-yl)-3-(3-(((1s,4r)-4-(3-((3R)-4-(3-(2,6-dioxopiperidin-3-yl)-1-methyl-1H-indazol-6-yl)-3-(trifluoromethyl)piperazin-1-yl)propyl)cyclohexyl)oxy)-2-methylphenyl)picolinate

To a solution of tert-butyl 6-(8-(benzo[d]thiazol-2-ylcarbamoyl)-3,4-dihydroisoquinolin-2(1H)-yl)-3-(2-methyl-3-(((1r,4r)-4-(3-oxopropyl)cyclohexyl)oxy)phenyl)picolinate (100 mg, 1 equiv., 137 μmol) and 3-(1-methyl-6-((R)-2-(trifluoromethyl)piperazin-1-yl)-1H-indazol-3-yl)piperidine-2,6-dione (59.5 mg, 1.1 equiv., 150 μmol) in DCM (2.00 mL) was added sodium triacetoxyborohydride (87.0 mg, 3 equiv., 410 μmol). The reaction mixture was stirred at room temperature for 18 h. The reaction mixture was diluted with DCM (75 mL), water (10 mL), and brine (10 mL). The organic layer was separated, dried over anhydrous sodium sulfate, and concentrated to provide crude tert-butyl 6-(8-(benzo[d]thiazol-2-ylcarbamoyl)-3,4-dihydroisoquinolin-2(1H)-yl)-3-(3-(((1s,4r)-4-(3-((3R)-4-(3-(2,6-dioxopiperidin-3-yl)-1-methyl-1H-indazol-6-yl)-3-(trifluoromethyl)piperazin-1-yl)propyl)cyclohexyl)oxy)-2-methylphenyl)picolinate (122 mg, 110 μmol, 80% yield), which was carried forward without further purification.

MS (ESI) m/z: 1110.6 [M+H]⁺.

### Step B. 6-(8-(benzo[d]thiazol-2-ylcarbamoyl)-3,4-dihydroisoquinolin-2(1H)-yl)-3-(3-(((1s,4r)-4-(3-((3R)-4-(3-(2,6-dioxopiperidin-3-yl)-1-methyl-1H-indazol-6-yl)-3-(trifluoromethyl)piperazin-1-yl)propyl)cyclohexyl)oxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(((1s,4r)-4-(3-((3R)-4-(3-(2,6-dioxopiperidin-3-yl)-1-methyl-1H-indazol-6-yl)-3-(trifluoromethyl)piperazin-1-yl)propyl)cyclohexyl)oxy)-2-methylphenyl)picolinate (122 mg, 1 equiv., 110 μmol) in DCM (10.00 mL) was added trifluoroacetic acid (5 mL). The reaction solution was stirred at room temperature for 48 h. The reaction solution was then concentrated and purified by reverse-phase HPLC to provide 6-(8-(benzo[d]thiazol-2-ylcarbamoyl)-3,4-dihydroisoquinolin-2(1H)-yl)-3-(3-(((1s,4r)-4-(3-((3R)-4-(3-(2,6-dioxopiperidin-3-yl)-1-methyl-1H-indazol-6-yl)-3-(trifluoromethyl)piperazin-1-yl)propyl)cyclohexyl)oxy)-2-methylphenyl)picolinic acid (64 mg, 61 μmol, 55% yield).

MS (ESI) m/z: 1054.4 [M+H]⁺

### Example 281. Preparation of Compound 301b

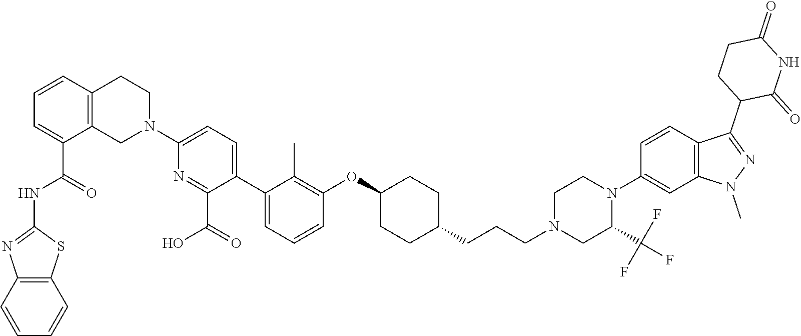 [SMILES: Cc1c(O[C@H]2CC[C@H](CCCN3CCN(c4ccc5c(C6CCC(=O)NC6=O)nn(C)c5c4)[C@H](C(F)(F)F)C3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-(3-((3S)-4-(3-(2,6-dioxopiperidin-3-yl)-1-methyl-1H-indazol-6-yl)-3-(trifluoromethyl)piperazin-1-yl)propyl)cyclohexyl)oxy)-2-methylphenyl)picolinic acid

### Step A. tert-butyl 6-(8-(benzo[d]thiazol-2-ylcarbamoyl)-3,4-dihydroisoquinolin-2(1H)-yl)-3-(3-(((1r,4r)-4-(3-((3S)-4-(3-(2,6-dioxopiperidin-3-yl)-1-methyl-1H-indazol-6-yl)-3-(trifluoromethyl)piperazin-1-yl)propyl)cyclohexyl)oxy)-2-methylphenyl)picolinate

To a solution of tert-butyl 6-(8-(benzo[d]thiazol-2-ylcarbamoyl)-3,4-dihydroisoquinolin-2(1H)-yl)-3-(2-methyl-3-(((1r,4r)-4-(3-oxopropyl)cyclohexyl)oxy)phenyl)picolinate (50 mg, 1 equiv., 68 mol) and 3-(1-methyl-6-((S)-2-(trifluoromethyl)piperazin-1-yl)-1H-indazol-3-yl)piperidine-2,6-dione (30 mg, 1.1 equiv., 75 μmol) in DCM (2.00 mL) was added sodium triacetoxyborohydride (43 mg, 3 equiv., 0.21 mmol). The reaction solution was stirred at room temperature for 72 h. The reaction solution was diluted with DCM (75 mL) and water (10 mL). The organic layer was separated, washed with brine (10 mL), dried over anhydrous sodium sulfate and concentrated to provide crude tert-butyl 6-(8-(benzo[d]thiazol-2-ylcarbamoyl)-3,4-dihydroisoquinolin-2(1H)-yl)-3-(3-(((1r,4r)-4-(3-((3S)-4-(3-(2,6-dioxopiperidin-3-yl)-1-methyl-1H-indazol-6-yl)-3-(trifluoromethyl)piperazin-1-yl)propyl)cyclohexyl)oxy)-2-methylphenyl)picolinate (84 mg), which was carried forward without further purification.

MS (ESI): m/z 1110.6 [M+H]⁺.

### Step B. 6-(8-(benzo[d]thiazol-2-ylcarbamoyl)-3,4-dihydroisoquinolin-2(1H)-yl)-3-(3-(((1r,4r)-4-(3-((3S)-4-(3-(2,6-dioxopiperidin-3-yl)-1-methyl-1H-indazol-6-yl)-3-(trifluoromethyl)piperazin-1-yl)propyl)cyclohexyl)oxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(((1r,4r)-4-(3-((3 S)-4-(3-(2,6-dioxopiperidin-3-yl)-1-methyl-1H-indazol-6-yl)-3-(trifluoromethyl)piperazin-1-yl)propyl)cyclohexyl)oxy)-2-methylphenyl)picolinate (84 mg, 1 equiv., 76 μmol) DCM (10.00 mL) was added trifluoroacetic acid (4.3 g, 2.9 mL, 500 equiv., 38 mmol). The reaction solution was stirred at room temperature for 18 h. The reaction solution was concentrated and purified by reverse-phase HPLC to provide 6-(8-(benzo[d]thiazol-2-ylcarbamoyl)-3,4-dihydroisoquinolin-2(1H)-yl)-3-(3-(((1r,4r)-4-(3-((3S)-4-(3-(2,6-dioxopiperidin-3-yl)-1-methyl-1H-indazol-6-yl)-3-(trifluoromethyl)piperazin-1-yl)propyl)cyclohexyl)oxy)-2-methylphenyl)picolinic acid (20 mg, 19 μmol, 25% yield).

MS (ESI): m/z 1054.6 [M+H]⁺

### Example 282. Preparation of Compound 339a

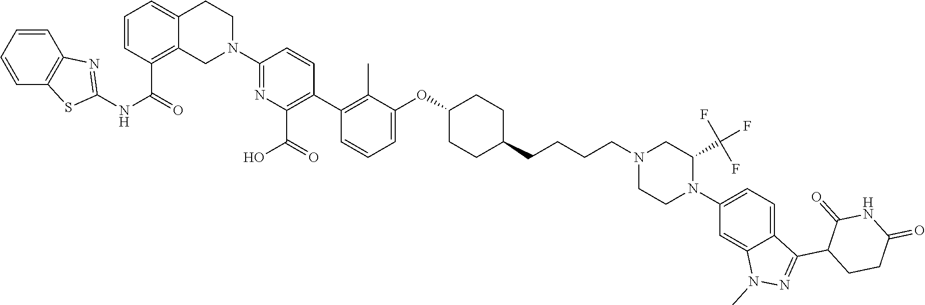 [SMILES: Cc1c(O[C@H]2CC[C@H](CCCCN3CCN(c4ccc5c(C6CCC(=O)NC6=O)nn(C)c5c4)[C@@H](C(F)(F)F)C3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(4-((3R)-4-(3-(2,6-dioxopiperidin-3-yl)-1-methyl-1H-indazol-6-yl)-3-(trifluoromethyl)piperazin-1-yl)butyl)cyclohexyl)oxy)-2-methylphenyl)picolinic acid

### Step A. Procedure for Preparation of tert-butyl (3R)-4-[3-(2,6-dibenzyloxy-3-pyridyl)-1-methyl-indazol-6-yl]-3-(trifluoromethyl)piperazine-1-carboxylate

A mixture of 6-bromo-3-(2,6-dibenzyloxy-3-pyridyl)-1-methyl-indazole (1.97 g, 3.93 mmol, 2.0 equiv.), tert-butyl (3R)-3-(trifluoromethyl)piperazine-1-carboxylate (500 mg, 1.97 mmol, 1.0 equiv.), Pd₂(dba)₃ (180.08 mg, 196.66 μmol, 0.1 equiv.), NaOtBu (377.99 mg, 3.93 mmol, 2.0 equiv.), and tritert-butylphosphane (795.74 mg, 393.32 μmol, 923.14 μL, 10% purity, 0.2 equiv.) in toluene (25 mL) was degassed and purged with N₂ three times. The mixture was stirred at 100° C. for 16 hours under N₂ atmosphere. The reaction mixture was filtered and concentrated under reduced pressure to give a residue. The residue was purified by flash silica gel chromatography (0˜30% ethyl acetate/petroleum ether) to afford tert-butyl (3R)-4-[3-(2,6-dibenzyloxy-3-pyridyl)-1-methyl-indazol-6-yl]-3-(trifluoromethyl)piperazine-1-carboxylate (1.4 g, 1.91 mmol, 97.21% yield) as a yellow oil. MS (ESI) m/z: 674.3 [M+H]⁺.

### Step B. Procedure for Preparation of tert-butyl (3R)-4-[3-(2,6-dioxo-3-piperidyl)-1-methyl-indazol-6-yl]-3-(trifluoromethyl)piperazine-1-carboxylate

To a solution of tert-butyl (3R)-4-[3-(2,6-dibenzyloxy-3-pyridyl)-1-methyl-indazol-6-yl]-3-(trifluoromethyl)piperazine-1-carboxylate (1.4 g, 2.08 mmol, 1.0 equiv.) in THE (10 mL) and EtOH (10 mL) was added Pd/C (653.33 mg, 613.92 μmol, 10% purity, 0.295 equiv.) and Pd(OH)₂ (653.33 mg, 4.65 mmol, 2.24 equiv.) under N₂ atmosphere. The suspension was degassed and purged with H₂ three times. The mixture was stirred under H₂ (15 Psi) at 40° C. for 16 hours. The reaction mixture was filtered and washed with THE (100 mL). The filtrate was concentrated to afford tert-butyl (3R)-4-[3-(2,6-dioxo-3-piperidyl)-1-methyl-indazol-6-yl]-3-(trifluoromethyl)piperazine-1-carboxylate (1.3 g, crude) as a brown solid, which was used into the next step without further purification. MS (ESI) m/z: 496.3 [M+H]⁺.

### Step C. Procedure for Preparation of 3-[1-methyl-6-[(2R)-2-(trifluoromethyl)piperazin-1-yl]indazol-3-yl]piperidine-2,6-dione

A solution of tert-butyl (3R)-4-[3-(2,6-dioxo-3-piperidyl)-1-methyl-indazol-6-yl]-3-(trifluoromethyl)piperazine-1-carboxylate (1.3 g, 2.62 mmol, 1.0 equiv.) in HCl/dioxane (20 mL) was stirred at 25° C. for 1 hour. The reaction mixture was filtered to afford 3-[1-methyl-6-[(2R)-2-(trifluoromethyl)piperazin-1-yl]indazol-3-yl]piperidine-2,6-dione (900 mg, crude) as a white solid, which was used into next step without further purification. MS (ESI) m/z: 396.1 [M+H]⁺.

### Step D. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(4-((3R)-4-(3-(2,6-dioxopiperidin-3-yl)-1-methyl-1H-indazol-6-yl)-3-(trifluoromethyl)piperazin-1-yl)butyl)cyclohexyl)oxy)-2-methylphenyl)picolinate

To a solution of tert-butyl 6-[8-(1,3-benzothiazol-2-ylcarbamoyl)-3,4-dihydro-1H-isoquinolin-2-yl]-3-[2-methyl-3-[4-(4-oxobutyl)cyclohexoxy]phenyl]pyridine-2-carboxylate (200 mg, 268.48 mol, 1.0 equiv.) and 3-[1-methyl-6-[(2R)-2-(trifluoromethyl)piperazin-1-yl]indazol-3-yl]piperidine-2,6-dione (159.23 mg, 402.72 μmol, 1.5 equiv.) in DCM (5 mL), was added NaBH(OAc)₃ (170.70 mg, 805.44 μmol, 3.0 equiv.). The mixture was stirred at 25° C. for 16 hours. The reaction mixture was diluted with H₂O (20 mL) and extracted with ethyl acetate (20 mL×3). The combined organic layers were washed with brine (30 mL), dried over Na₂SO₄, filtered, and concentrated under reduced pressure to afford tert-butyl 6-(8-(benzo[d]thiazol-2-ylcarbamoyl)-3,4-dihydroisoquinolin-2(1H)-yl)-3-(3-(((1s,4r)-4-(4-((3R)-4-(3-(2,6-dioxopiperidin-3-yl)-1-methyl-1H-indazol-6-yl)-3-(trifluoromethyl)piperazin-1-yl)butyl)cyclohexyl)oxy)-2-methylphenyl)picolinate (300 mg, crude) as a yellow solid, which was used into next step without further purification. MS (ESI) m/z: 1124.6 [M+H]⁺.

### Step E. Procedure for Preparation of 6-(8-(benzo[d]thiazol-2-ylcarbamoyl)-3,4-dihydroisoquinolin-2(1H)-yl)-3-(3-(((1s,4r)-4-(4-((3R)-4-(3-(2,6-dioxopiperidin-3-yl)-1-methyl-1H-indazol-6-yl)-3-(trifluoromethyl)piperazin-1-yl)butyl)cyclohexyl)oxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(((1s,4r)-4-(4-((3R)-4-(3-(2,6-dioxopiperidin-3-yl)-1-methyl-1H-indazol-6-yl)-3-(trifluoromethyl)piperazin-1-yl)butyl)cyclohexyl)oxy)-2-methylphenyl)picolinate (300 mg, 266.83 μmol, 1.0 equiv.) in DCM (1 mL) was added TFA (4.60 g, 40.39 mmol, 3.00 mL, 151.36 equiv.). The mixture was stirred at 30° C. for 2 hours. The reaction mixture was concentrated under reduced pressure to give a residue. The residue was purified by prep-HPLC to afford 6-(8-(benzo[d]thiazol-2-ylcarbamoyl)-3,4-dihydroisoquinolin-2(1H)-yl)-3-(3-(((1s,4r)-4-(4-((3R)-4-(3-(2,6-dioxopiperidin-3-yl)-1-methyl-1H-indazol-6-yl)-3-(trifluoromethyl)piperazin-1-yl)butyl)cyclohexyl)oxy)-2-methylphenyl)picolinic acid (118.19 mg, 109.27 μmol, 40.95% yield) as a white solid. MS (ESI) m/z: 1068.6 [M+H]⁺. ¹H NMR (400 MHz, DMSO-d₆) δ=12.86 (s, 1H), 12.64-12.52 (m, 1H), 10.85 (s, 1H), 8.03 (d, J=8.0 Hz, 1H), 7.79 (d, J=8.0 Hz, 1H), 7.62 (d, J=7.2 Hz, 1H), 7.54-7.41 (m, 4H), 7.40-7.32 (m, 2H), 7.11-6.88 (m, 5H), 6.61 (d, J=7.2 Hz, 1H), 4.98 (s, 2H), 4.34-4.15 (m, 2H), 3.95-3.87 (m, 5H), 3.49-3.39 (m, 2H), 3.23-3.16 (m, 1H), 3.02 (t, J=6.0 Hz, 2H), 2.99-2.90 (m, 1H), 2.71-2.53 (m, 3H), 2.44-2.21 (m, 4H), 2.20-1.98 (m, 4H), 1.93-1.86 (m, 3H), 1.78 (d, J=12.0 Hz, 2H), 1.50-1.20 (m, 9H), 1.05 (d, J=12.4 Hz, 2H)

### Example 283. Preparation of Compound 340a

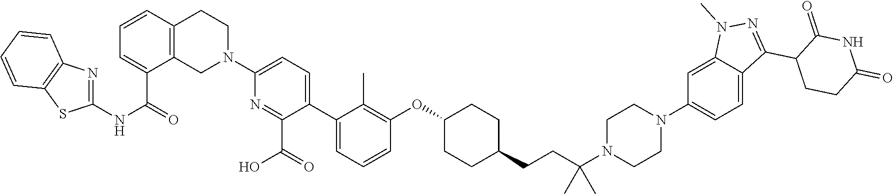 [SMILES: Cc1c(O[C@H]2CC[C@H](CCC(C)(C)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)piperazin-1-yl)-3-methylbutyl)cyclohexyl)oxy)-2-methylphenyl)picolinic acid

### Step A. Procedure for Preparation of 1-(4-(3-(2,6-bis(benzyloxy)pyridin-3-yl)-1-methyl-1H-indazol-6-yl)piperazin-1-yl)-3-((1r,4r)-4-(3-bromo-2-methylphenoxy)cyclohexyl)propan-1-one

A mixture of 3-((1r,4r)-4-(3-bromo-2-methylphenoxy)cyclohexyl)propanoic acid (800 mg, 2.34 mmol, 1.0 equiv.), 3-(2,6-dibenzyloxy-3-pyridyl)-1-methyl-6-piperazin-1-yl-indazole (1.42 g, 2.81 mmol, 1.2 equiv.), EDCI (674.14 mg, 3.52 mmol, 1.5 equiv.) in pyridine (10 mL) was stirred at 40° C. for 2 hours. The mixture was then concentrated under reduced pressure to give a residue. The residue was purified by flash silica gel chromatography (0˜70% ethyl acetate/petroleum ether) to give 1-(4-(3-(2,6-bis(benzyloxy)pyridin-3-yl)-1-methyl-1H-indazol-6-yl)piperazin-1-yl)-3-((1r,4r)-4-(3-bromo-2-methylphenoxy)cyclohexyl)propan-1-one (2.7 g, crude) as a yellow oil. MS (ESI) m/z: 828.4 [M+H]⁺. ¹H NMR (400 MHz, DMSO-d₆) δ=7.89 (d, J=8.0 Hz, 1H), 7.53 (d, J=9.2 Hz, 1H), 7.48-7.28 (m, 10H), 7.15-7.00 (m, 3H), 6.89-6.82 (m, 2H), 6.56 (d, J=8.0 Hz, 1H), 5.76-5.74 (m, 1H), 5.46-5.40 (m, 4H), 3.97 (s, 3H), 3.66-3.58 (m, 4H), 3.24-3.15 (m, 4H), 2.21 (s, 2H), 1.99 (s, 3H), 1.50-1.26 (m, 6H), 1.22-1.13 (m, 4H), 1.11-0.98 (m, 2H)

### Step B. Procedure for Preparation of 3-(2,6-bis(benzyloxy)pyridin-3-yl)-6-(4-(4-((1r,4s)-4-(3-bromo-2-methylphenoxy)cyclohexyl)-2-methylbutan-2-yl)piperazin-1-yl)-1-methyl-1H-indazole

A solution of 1-(4-(3-(2,6-bis(benzyloxy)pyridin-3-yl)-1-methyl-1H-indazol-6-yl)piperazin-1-yl)-3-((1r,4r)-4-(3-bromo-2-methylphenoxy)cyclohexyl)propan-1-one (2.7 g, 3.26 mmol, 1.0 equiv.), and ZrCl₄ (835.05 mg, 3.58 mmol, 298.23 μL, 1.1 equiv.) in THE (30 mL) was degassed and purged with N₂ three times. The mixture was stirred at −10° C. for 1 hour under N₂ atmosphere. Then MeMgBr (3 M, 7.60 mL, 7.0 equiv.) was added dropwise, and the mixture was warmed to 25° C. The resulting mixture was stirred for 1 hour. After completion, the reaction mixture was poured into saturated aq. NH₄Cl (50 mL), and then extracted with ethyl acetate (50 mL×3). The combined organic layers were washed with brine (50 mL), dried over anhydrous sodium sulfate, filtered, and concentrated under reduced pressure to give a residue. The residue was purified by flash silica gel chromatography (0˜10% DCM/MeOH) to give 3-(2,6-bis(benzyloxy)pyridin-3-yl)-6-(4-(4-((1r,4s)-4-(3-bromo-2-methylphenoxy)cyclohexyl)-2-methylbutan-2-yl)piperazin-1-yl)-1-methyl-1H-indazole (1.3 g, 1.54 mmol, 47.34% yield) as a yellow oil. MS (ESI) m/z: 844.4 [M+H]⁺.

### Step C. Procedure for Preparation of 3-(2,6-bis(benzyloxy)pyridin-3-yl)-1-methyl-6-(4-(2-methyl-4-((1r,4s)-4-(2-methyl-3-(4,4,5,5-tetramethyl-1,3,2-dioxaborolan-2-yl)phenoxy)cyclohexyl)butan-2-yl)piperazin-1-yl)-1H-indazole

A mixture of 3-(2,6-bis(benzyloxy)pyridin-3-yl)-6-(4-(4-((1r,4s)-4-(3-bromo-2-methylphenoxy)cyclohexyl)-2-methylbutan-2-yl)piperazin-1-yl)-1-methyl-1H-indazole (1.2 g, 1.42 mmol, 1.0 equiv.), 4,4,4′,4′,5,5,5′,5′-octamethyl-2,2′-bi(1,3,2-dioxaborolane) (1.08 g, 4.27 mmol, 3.0 equiv.), KOAc (209.58 mg, 2.14 mmol, 1.5 equiv.), and Pd(dppf)Cl₂ (312.51 mg, 427.10 mol, 0.3 equiv.) in dioxane (15 mL) was degassed and purged with N₂ three times. The mixture was stirred at 90° C. for 16 hours under N₂ atmosphere. The reaction mixture was filtered and concentrated under reduced pressure to give a residue. The residue was purified by flash silica gel chromatography (0˜10% ethyl acetate/petroleum ether) to give 3-(2,6-bis(benzyloxy)pyridin-3-yl)-1-methyl-6-(4-(2-methyl-4-((1r,4s)-4-(2-methyl-3-(4,4,5,5-tetramethyl-1,3,2-dioxaborolan-2-yl)phenoxy)cyclohexyl)butan-2-yl)piperazin-1-yl)-1H-indazole (1.6 g, crude) as a yellow oil. MS (ESI) m/z: 890.8 [M+H]⁺. ¹H NMR (400 MHz, DMSO-d₆) δ=7.95-7.92 (m, 1H), 7.91-7.86 (m, 1H), 7.48-7.45 (m, 2H), 7.42-7.28 (m, 8H), 7.20-7.16 (m, 1H), 7.11-7.06 (m, 1H), 6.85-6.80 (m, 1H), 6.57 (d, J=8.0 Hz, 1H), 5.76-5.74 (m, 2H), 5.47-5.40 (m, 4H), 4.24-4.13 (m, 1H), 3.97 (s, 3H), 3.94-3.92 (m, 1H), 3.63-3.54 (m, 1H), 3.16 (s, 3H), 2.72-2.56 (m, 3H), 2.30 (s, 2H), 2.13-2.00 (m, 2H), 1.91 (s, 2H), 1.87-1.73 (m, 2H), 1.48-1.32 (m, 4H), 1.30-1.28 (m, 8H), 1.17-1.15 (m, 11H), 1.08-1.06 (m, 4H)

### Step D. Procedure for Preparation of 3-(1-methyl-6-(4-(2-methyl-4-((1r,4s)-4-(2-methyl-3-(4,4,5,5-tetramethyl-1,3,2-dioxaborolan-2-yl)phenoxy)cyclohexyl)butan-2-yl)piperazin-1-yl)-1H-indazol-3-yl)piperidine-2,6-dione

To a solution of 3-(2,6-bis(benzyloxy)pyridin-3-yl)-1-methyl-6-(4-(2-methyl-4-((1r,4s)-4-(2-methyl-3-(4,4,5,5-tetramethyl-1,3,2-dioxaborolan-2-yl)phenoxy)cyclohexyl)butan-2-yl)piperazin-1-yl)-1H-indazole (800 mg, 898.91 μmol, 1.0 equiv.) in EtOH (10 mL) was added Pd/C (400 mg, 375.87 μmol, 10% purity, 0.418 equiv.) and Pd(OH)₂ (400 mg, 569.66 μmol, 20% purity, 0.634 equiv.). The mixture was stirred at 50° C. for 16 hours. The combined organic layers were filtered and concentrated under reduced pressure to give 3-(1-methyl-6-(4-(2-methyl-4-((1r,4s)-4-(2-methyl-3-(4,4,5,5-tetramethyl-1,3,2-dioxaborolan-2-yl)phenoxy)cyclohexyl)butan-2-yl)piperazin-1-yl)-1H-indazol-3-yl)piperidine-2,6-dione (300 mg, 421.50 μmol, 46.89% yield) as a yellow oil. MS (ESI) m/z: 712.5 [M+H]⁺.

### Step E. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)piperazin-1-yl)-3-methylbutyl)cyclohexyl)oxy)-2-methylphenyl)picolinate

A mixture of 3-(1-methyl-6-(4-(2-methyl-4-((1r,4s)-4-(2-methyl-3-(4,4,5,5-tetramethyl-1,3,2-dioxaborolan-2-yl)phenoxy)cyclohexyl)butan-2-yl)piperazin-1-yl)-1H-indazol-3-yl)piperidine-2,6-dione (271.87 mg, 381.98 μmol, 1.2 equiv.), tert-butyl 6-[8-(1,3-benzothiazol-2-ylcarbamoyl)-3,4-dihydro-1H-isoquinolin-2-yl]-3-bromo-pyridine-2-carboxylate (180 mg, 318.31 μmol, 1 equiv.), KF (1.5 M, 636.63 μL, 3.0 equiv.), and [2-(2-aminophenyl)phenyl]palladium(1⁺)bis(1-adamantyl)-butyl-phosphane methanesulfonate (46.36 mg, 63.66 μmol, 0.2 equiv.) in dioxane (0.5 mL) was degassed and purged with N₂ three times. The mixture was stirred at 100° C. for 1 hour under N₂ atmosphere. The combined organic layers were filtered and concentrated under reduced pressure to give a residue. The residue was purified by flash silica gel chromatography (0˜100% petroleum ether/ethyl acetate) to give tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)piperazin-1-yl)-3-methylbutyl)cyclohexyl)oxy)-2-methylphenyl)picolinate (110 mg, 102.77 μmol, 32.29% yield) as a yellow solid. MS (ESI) m/z: 1070.6 [M+H]⁺.

### Step F. Procedure for Preparation of 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)piperazin-1-yl)-3-methylbutyl)cyclohexyl)oxy)-2-methylphenyl)picolinic acid

A solution of tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)piperazin-1-yl)-3-methylbutyl)cyclohexyl)oxy)-2-methylphenyl)picolinate (100 mg, 93.43 μmol, 1.0 equiv.) in TFA (1 mL) and DCM (1 mL) was stirred at 25° C. for 2 hours. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)piperazin-1-yl)-3-methylbutyl)cyclohexyl)oxy)-2-methylphenyl)picolinic acid (28.38 mg, 27.07 μmol, 28.98% yield) as a white solid. MS (ESI) m/z: 1014.6 [M+H]⁺. ¹H NMR (400 MHz, DMSO-d₆) δ=13.06-12.47 (m, 2H), 10.97-10.73 (m, 1H), 8.06-8.01 (m, 1H), 7.79 (d, J=7.6 Hz, 1H), 7.65-7.59 (m, 1H), 7.50-7.34 (m, 6H), 7.10-7.04 (m, 1H), 6.98-6.91 (m, 3H), 6.88-6.80 (m, 1H), 6.61 (d, J=7.6 Hz, 1H), 4.98 (s, 2H), 4.32-4.14 (m, 2H), 3.95-3.87 (m, 5H), 3.31-3.29 (m, 7H), 3.05-3.00 (m, 2H), 2.68-2.65 (m, 2H), 2.35-2.28 (m, 2H), 2.19-2.04 (m, 4H), 1.87 (s, 3H), 1.84-1.77 (m, 2H), 1.49-1.22 (m, 8H), 1.12-1.01 (m, 6H)

### Example 284. Preparation of Compound 341a

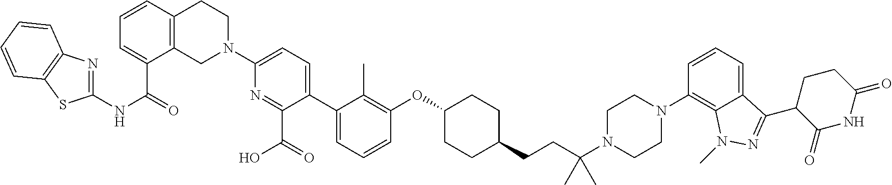 [SMILES: Cc1c(O[C@H]2CC[C@H](CCC(C)(C)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((is, 4r)-4-(3-(4-(3-(2,6-dioxopiperidin-3-yl)-1-methyl-1H-indazol-7-yl)piperazin-1-yl)-3-methylbutyl)cyclohexyl)oxy)-2-methylphenyl)picolinic acid

### Step A. Procedure for Preparation of 3-((1r,4r)-4-(3-bromo-2-methylphenoxy)cyclohexyl)propanoic acid

A mixture of ethyl 3-((1r, 4r)-4-(3-bromo-2-methylphenoxy)cyclohexyl)propanoate (3 g, 8.12 mmol, 1.0 equiv.) and LiOH·H₂O (1.70 g, 40.62 mmol, 5.0 equiv.) in THE (30 mL) and H₂O (10 mL) was stirred at 25° C. for 16 hours. The reaction mixture was diluted with H₂O (5 mL) and concentrated as a turbid liquid. Then the pH of the turbid liquid was adjusted to pH˜3 with 1 N HCl (5 mL). The resulting solution was extracted with DCM/MeOH (20:1) and the combined organic layers were filtered and concentrated to afford 3-((1r,4r)-4-(3-bromo-2-methylphenoxy)cyclohexyl)propanoic acid (3 g, crude) as a yellow oil which was used in the next step without further purification.

### Step B. Procedure for Preparation of 1-(4-(3-(2,6-bis(benzyloxy)pyridin-3-yl)-1-methyl-1H-indazol-7-yl)piperazin-1-yl)-3-((1r, 4r)-4-(3-bromo-2-methylphenoxy)cyclohexyl)propan-1-one

A mixture of 3-((1r,4r)-4-(3-bromo-2-methylphenoxy)cyclohexyl)propanoic acid (1.60 g, 4.68 mmol, 1.1 equiv.), 3-(2,6-dibenzyloxy-3-pyridyl)-1-methyl-7-piperazin-1-yl-indazole (2.15 g, 4.25 mmol, 1.0 equiv.), EDCI (1.22 g, 6.38 mmol, 1.5 equiv.) in pyridine (5 mL) was stirred at 25° C. for 2 hours. The combined organic layers were filtered and concentrated under reduced pressure to give a residue. The residue was purified by flash silica gel chromatography (0˜60% ethyl acetate/petroleum ether) to give 1-(4-(3-(2,6-bis(benzyloxy)pyridin-3-yl)-1-methyl-1H-indazol-7-yl)piperazin-1-yl)-3-((1r, 4r)-4-(3-bromo-2-methylphenoxy)cyclohexyl)propan-1-one (1.8 g, 2.17 mmol, 51.07% yield) as a white solid. MS (ESI) m/z: 830.4 [M+H]⁺.

### Step C. Procedure for Preparation of 3-(2,6-bis(benzyloxy)pyridin-3-yl)-7-(4-(4-((1r, 4s)-4-(3-bromo-2-methylphenoxy)cyclohexyl)-2-methylbutan-2-yl)piperazin-1-yl)-1-methyl-1H-indazole

A mixture of 1-(4-(3-(2,6-bis(benzyloxy)pyridin-3-yl)-1-methyl-1H-indazol-7-yl)piperazin-1-yl)-3-((1r, 4r)-4-(3-bromo-2-methylphenoxy)cyclohexyl)propan-1-one (1.8 g, 2.17 mmol, 1.0 equiv.) and ZrCl₄ (556.70 mg, 2.39 mmol, 198.82 μL, 1.1 equiv.) in THE (20 mL) was degassed and purged with N₂ three times. The mixture was stirred at −10° C. for 1 hour under N₂ atmosphere, followed by the addition of MeMgBr (3 M, 5.07 mL, 7 equiv.). The reaction mixture was stirred at 25° C. for 1 hour under N₂ atmosphere. After completion, the reaction mixture was poured into saturated aq. NH₄Cl (50 mL), and extracted with ethyl acetate 150 mL (50 mL×3). The combined organic layers were washed with brine (50 mL), dried over anhydrous sodium sulfate, filtered, and concentrated under reduced pressure to give a residue. The residue was purified by flash silica gel chromatography (0˜10% DCM/MeOH) to give 3-(2,6-bis(benzyloxy)pyridin-3-yl)-7-(4-(4-((1r, 4s)-4-(3-bromo-2-methylphenoxy)cyclohexyl)-2-methylbutan-2-yl)piperazin-1-yl)-1-methyl-1H-indazole (1.2 g, 1.42 mmol, 65.55% yield) as a yellow solid. MS (ESI) m/z: 844.4 [M+H]⁺.

### Step D. Procedure for Preparation of 3-(2,6-bis(benzyloxy)pyridin-3-yl)-1-methyl-7-(4-(2-methyl-4-((1r,4s)-4-(2-methyl-3-(4,4,5,5-tetramethyl-1,3,2-dioxaborolan-2-yl)phenoxy)cyclohexyl)butan-2-yl)piperazin-1-yl)-1H-indazole

A mixture of 3-(2,6-bis(benzyloxy)pyridin-3-yl)-7-(4-(4-((1r, 4s)-4-(3-bromo-2-methylphenoxy)cyclohexyl)-2-methylbutan-2-yl)piperazin-1-yl)-1-methyl-1H-indazole (1.2 g, 1.42 mmol, 1.0 equiv.), 4,4,4′,4′,5,5,5′,5′-octamethyl-2,2′-bi(1,3,2-dioxaborolane) (1.08 g, 4.27 mmol, 3.0 equiv.), KOAc (209.58 mg, 2.14 mmol, 1.5 equiv.), Pd(dppf)Cl₂ (312.51 mg, 427.10 mol, 0.3 equiv.) in dioxane (12 mL) was degassed and purged with N₂ three times. The mixture was stirred at 90° C. for 2 hours under N₂ atmosphere. The reaction mixture was filtered and concentrated under reduced pressure to give a residue. The residue was purified by flash silica gel chromatography (0˜10% ethyl acetate/petroleum ether) to give 3-(2,6-bis(benzyloxy)pyridin-3-yl)-1-methyl-7-(4-(2-methyl-4-((1r,4s)-4-(2-methyl-3-(4,4,5,5-tetramethyl-1,3,2-dioxaborolan-2-yl)phenoxy)cyclohexyl)butan-2-yl)piperazin-1-yl)-1H-indazole (1.6 g, crude) as a yellow oil. MS (ESI) m/z: 890.8 [M+H]⁺.

### Step E. Procedure for Preparation of 3-(1-methyl-7-(4-(2-methyl-4-((1r,4s)-4-(2-methyl-3-(4,4,5,5-tetramethyl-1,3,2-dioxaborolan-2-yl)phenoxy)cyclohexyl)butan-2-yl)piperazin-1-yl)-1H-indazol-3-yl)piperidine-2,6-dione

A solution of 3-(2,6-bis(benzyloxy)pyridin-3-yl)-1-methyl-7-(4-(2-methyl-4-((1r,4s)-4-(2-methyl-3-(4,4,5,5-tetramethyl-1,3,2-dioxaborolan-2-yl)phenoxy)cyclohexyl)butan-2-yl)piperazin-1-yl)-1H-indazole (800 mg, 898.91 μmol, 1 equiv.), Pd/C (400 mg, 375.87 μmol, 10% purity, 0.418 equiv.), and Pd(OH)₂ (400 mg, 569.66 μmol, 20% purity, 0.634 equiv.) in EtOH (10 mL) was degassed and purged with N₂ three times. The mixture was stirred at 50° C. for 16 hours under N₂ atmosphere. The combined organic layers were filtered and concentrated under reduced pressure to give 3-(1-methyl-7-(4-(2-methyl-4-((1r,4s)-4-(2-methyl-3-(4,4,5,5-tetramethyl-1,3,2-dioxaborolan-2-yl)phenoxy)cyclohexyl)butan-2-yl)piperazin-1-yl)-1H-indazol-3-yl)piperidine-2,6-dione (350 mg, 491.75 μmol, 54.71% yield) was obtained as a yellow oil which was used in the next step without further purification. MS (ESI) m/z: 712.5 [M+H]⁺.

### Step F. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-7-yl)piperazin-1-yl)-3-methylbutyl)cyclohexyl)oxy)-2-methylphenyl)picolinate

A mixture of 3-(1-methyl-7-(4-(2-methyl-4-((1r,4s)-4-(2-methyl-3-(4,4,5,5-tetramethyl-1,3,2-dioxaborolan-2-yl)phenoxy)cyclohexyl)butan-2-yl)piperazin-1-yl)-1H-indazol-3-yl)piperidine-2,6-dione (151.04 mg, 212.21 μmol, 1.2 equiv.), tert-butyl 6-[8-(1,3-benzothiazol-2-ylcarbamoyl)-3,4-dihydro-1H-isoquinolin-2-yl]-3-bromo-pyridine-2-carboxylate (100 mg, 176.84 μmol, 1.0 equiv.), KF (1.5 M, 353.68 μL, 3.0 equiv.), and [2-(2-aminophenyl)phenyl]palladium(1⁺) bis(1-adamantyl)-butyl-phosphane methanesulfonate (25.76 mg, 35.37 μmol, 0.2 equiv.) in dioxane (2 mL) was degassed and purged with N₂ three times. The mixture was stirred at 100° C. for 1 hour under N₂ atmosphere in a microwave reactor. The combined organic layers were filtered and concentrated under reduced pressure to give a residue. The residue was purified by flash silica gel chromatography (0˜100% ethyl acetate/petroleum ether) to give tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-7-yl)piperazin-1-yl)-3-methylbutyl)cyclohexyl)oxy)-2-methylphenyl)picolinate (65 mg, 60.73 μmol, 34.34% yield) as a yellow solid. MS (ESI) m/z: 1070.6 [M+H]⁺.

### Step G. Procedure for Preparation of 6-(8-(benzo[d]thiazol-2-ylcarbamoyl)-3,4-dihydroisoquinolin-2(1H)-yl)-3-(3-(((1s, 4r)-4-(3-(4-(3-(2,6-dioxopiperidin-3-yl)-1-methyl-1H-indazol-7-yl)piperazin-1-yl)-3-methylbutyl)cyclohexyl)oxy)-2-methylphenyl)picolinic acid

A solution of tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-7-yl)piperazin-1-yl)-3-methylbutyl)cyclohexyl)oxy)-2-methylphenyl)picolinate (60 mg, 56.06 μmol, 1.0 equiv.) in TFA (1 mL) and DCM (1 mL) was stirred at 25° C. for 16 hours. The reaction mixture was filtered and concentrated under reduced pressure to give a residue The residue was purified by prep-HPLC to give 6-(8-(benzo[d]thiazol-2-ylcarbamoyl)-3,4-dihydroisoquinolin-2(1H)-yl)-3-(3-(((is, 4r)-4-(3-(4-(3-(2,6-dioxopiperidin-3-yl)-1-methyl-1H-indazol-7-yl)piperazin-1-yl)-3-methylbutyl)cyclohexyl)oxy)-2-methylphenyl)picolinic acid (24.74 mg, 23.73 μmol, 42.34% yield) as a white solid. MS (ESI) m/z: 1014.6 [M+H]⁺. ¹H NMR (400 MHz, DMSO-d₆) δ=12.98-12.77 (m, 1H), 12.69-12.33 (m, 1H), 10.95-10.81 (m, 1H), 8.03 (d, J=8.0 Hz, 1H), 7.79 (d, J=7.6 Hz, 1H), 7.62 (d, J=8.0 Hz, 1H), 7.49-7.32 (m, 6H), 7.10-6.90 (m, 5H), 6.61 (d, J=7.6 Hz, 1H), 5.06-4.90 (m, 2H), 4.37-4.31 (m, 1H), 4.25 (s, 3H), 3.98-3.84 (m, 2H), 3.29-3.27 (m, 6H), 3.05-3.01 (m, 2H), 2.70-2.59 (m, 4H), 2.36-2.30 (m, 2H), 2.23-2.03 (m, 4H), 1.89-1.79 (m, 5H), 1.41-1.23 (m, 8H), 1.16-0.97 (m, 6H)

### Example 285. Preparation of Compound 342a

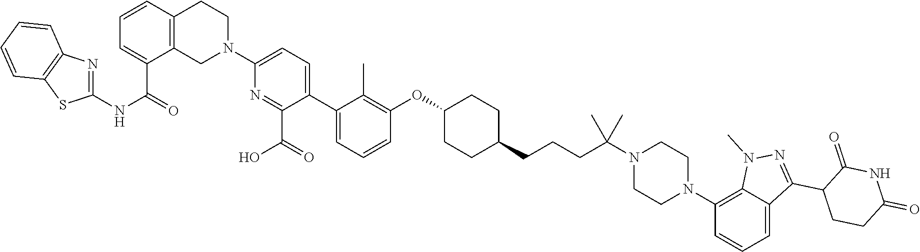 [SMILES: Cc1c(O[C@H]2CC[C@H](CCCC(C)(C)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-7-yl)piperazin-1-yl)-4-methylpentyl)cyclohexyl)oxy)-2-methylphenyl)picolinic acid

### Step A. Procedure for Preparation of 1-(4-(3-(2,6-bis(benzyloxy)pyridin-3-yl)-1-methyl-1H-indazol-7-yl)piperazin-1-yl)-4-((1r,4s)-4-(3-bromo-2-methylphenoxy)cyclohexyl)butan-1-one

To a solution of 4-((1r,4s)-4-(3-bromo-2-methylphenoxy)cyclohexyl)butanoic acid (380 mg, 1.07 mmol, 1 equiv.) in DMF (10 mL) was added HATU (488.04 mg, 1.28 mmol, 1.2 equiv.) and TEA (324.70 mg, 3.21 mmol, 446.63 μL, 3 equiv.). Afterwards, 3-(2,6-bis(benzyloxy)pyridin-3-yl)-1-methyl-7-(piperazin-1-yl)-1H-indazole (648.97 mg, 1.28 mmol, 1.2 equiv.) was added to the mixture. The mixture was stirred at 40° C. for 12 hours. The reaction mixture was quenched by addition H₂O 20 mL. The reaction mixture was filtered, and the filter cake was washed with 10 mL of water and diluted in DCM (30 mL). The combined organic layer was dried over Na₂SO₄, filtered, and concentrated under reduced pressure to give a residue. The residue was purified by flash silica gel chromatography (0˜60% ethyl acetate/petroleum ether) to give 1-(4-(3-(2,6-bis(benzyloxy)pyridin-3-yl)-1-methyl-1H-indazol-7-yl)piperazin-1-yl)-4-((1r,4s)-4-(3-bromo-2-methylphenoxy)cyclohexyl)butan-1-one (900 mg, crude) as a yellow solid. MS (ESI) m/z: 844.4 [M+H]⁺.

### Step B. Procedure for Preparation of 3-(2,6-bis(benzyloxy)pyridin-3-yl)-7-(4-(5-((1r,4s)-4-(3-bromo-2-methylphenoxy)cyclohexyl)-2-methylpentan-2-yl)piperazin-1-yl)-1-methyl-1H-indazole

A mixture of 1-(4-(3-(2,6-bis(benzyloxy)pyridin-3-yl)-1-methyl-1H-indazol-7-yl)piperazin-1-yl)-4-((1r,4s)-4-(3-bromo-2-methylphenoxy)cyclohexyl)butan-1-one (600 mg, 711.86 μmol, 1 equiv.), and ZrCl₄ (199.07 mg, 854.23 μmol, 71.10 μL, 1.2 equiv.) in THE (20 mL) was degassed and purged with N₂ three times. MeMgBr (2.5 M, 1.99 mL, 7 equiv.) was then added dropwise at −10° C. The mixture was stirred at −10° C. for 15 min under N₂ atmosphere. After completion, the reaction mixture was poured into saturated aq. NH₄Cl (40 mL), and extracted was ethyl acetate (40 mL). The combined organic layers were washed with brine (40 mL), dried over anhydrous sodium sulfate, filtered, and concentrated under reduced pressure to give a residue. The residue was purified by flash silica gel chromatography (0˜60% ethyl acetate/petroleum ether) to give 3-(2,6-bis(benzyloxy)pyridin-3-yl)-7-(4-(5-((1r,4s)-4-(3-bromo-2-methylphenoxy)cyclohexyl)-2-methylpentan-2-yl)piperazin-1-yl)-1-methyl-1H-indazole (160 mg, 179.24 μmol, 25.18% yield) as a yellow oil. MS (ESI) m/z: 858.5 [M+H]⁺. ¹H NMR (400 MHz, DMSO-d₆) δ=7.85 (d, J=8.0 Hz, 1H), 7.50-7.43 (m, 2H), 7.43-7.24 (m, 9H), 7.16-7.11 (m, 1H), 7.09-7.04 (m, 1H), 7.01 (d, J=7.8 Hz, 2H), 6.95-6.90 (m, 1H), 6.58 (d, J=8.2 Hz, 1H), 5.76 (s, 1H), 5.42 (d, J=5.6 Hz, 4H), 4.32 (s, 3H), 4.27-4.19 (m, 1H), 2.98-2.70 (m, 4H), 2.20 (s, 3H), 2.08-2.02 (m, 2H), 1.82-1.74 (m, 2H), 1.43-1.26 (m, 8H), 1.17 (t, J=8.0 Hz, 4H), 1.07-0.99 (m, 8H).

### Step C. Procedure for Preparation of 3-(2,6-bis(benzyloxy)pyridin-3-yl)-1-methyl-7-(4-(2-methyl-5-((1r,4s)-4-(2-methyl-3-(4,4,5,5-tetramethyl-1,3,2-dioxaborolan-2-yl)phenoxy)cyclohexyl)pentan-2-yl)piperazin-1-yl)-1H-indazole

A mixture of 3-(2,6-bis(benzyloxy)pyridin-3-yl)-7-(4-(5-((1r,4s)-4-(3-bromo-2-methylphenoxy)cyclohexyl)-2-methylpentan-2-yl)piperazin-1-yl)-1-methyl-1H-indazole (160 mg, 186.71 μmol, 1 equiv.), 4,4,4′,4′,5,5,5′,5′-octamethyl-2,2′-bi(1,3,2-dioxaborolane) (71.12 mg, 280.07 μmol, 1.5 equiv.), KOAc (91.62 mg, 933.57 μmol, 5 equiv.), and Pd(dppf)Cl₂ (27.32 mg, 37.34 μmol, 0.2 equiv.) in dioxane (3 mL) was degassed and purged with N₂ three times. The mixture was stirred at 90° C. for 12 hours under N₂ atmosphere. The reaction mixture was filtered and concentrated under reduced pressure to give a residue. The residue was purified by flash silica gel chromatography (0˜60% ethyl acetate/petroleum ether) to give 3-(2,6-bis(benzyloxy)pyridin-3-yl)-1-methyl-7-(4-(2-methyl-5-((1r,4s)-4-(2-methyl-3-(4,4,5,5-tetramethyl-1,3,2-dioxaborolan-2-yl)phenoxy)cyclohexyl)pentan-2-yl)piperazin-1-yl)-1H-indazole (150 mg, 152.66 μmol, 81.76% yield) as a yellow oil. MS (ESI) m/z: 904.6 [M+H]⁺.

### Step D. Procedure for Preparation of 3-(1-methyl-7-(4-(2-methyl-5-((1r,4s)-4-(2-methyl-3-(4,4,5,5-tetramethyl-1,3,2-dioxaborolan-2-yl)phenoxy)cyclohexyl)pentan-2-yl)piperazin-1-yl)-1H-indazol-3-yl)piperidine-2,6-dione

A mixture of 3-(2,6-bis(benzyloxy)pyridin-3-yl)-1-methyl-7-(4-(2-methyl-5-((1r,4s)-4-(2-methyl-3-(4,4,5,5-tetramethyl-1,3,2-dioxaborolan-2-yl)phenoxy)cyclohexyl)pentan-2-yl)piperazin-1-yl)-1H-indazole (150 mg, 152.66 μmol, 1 equiv.), Pd/C (90 mg, 84.57 μmol, 10% purity, 0.554 equiv.), and Pd(OH)₂ (90 mg, 128.17 μmol, 20% purity, 0.84 equiv.) in EtOH (4 mL) and THE (4 mL) was degassed and purged with H₂ three times. The mixture was stirred at 40° C. for 12 hours under H₂ atmosphere. The mixture was filtered and washed with THE (50 mL), DCM (50 mL), and MeOH (20 mL). The filtrate was concentrated under reduced pressure to give a residue. The residue was purified by prep-TLC (SiO₂, petroleum ether:ethyl acetate=0:1) to give 3-(1-methyl-7-(4-(2-methyl-5-((1r,4s)-4-(2-methyl-3-(4,4,5,5-tetramethyl-1,3,2-dioxaborolan-2-yl)phenoxy)cyclohexyl)pentan-2-yl)piperazin-1-yl)-1H-indazol-3-yl)piperidine-2,6-dione (110 mg, 137.92 μmol, 90.35% yield) as a yellow solid. MS (ESI) m/z: 726.5 [M+H]⁺.

### Step E. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-7-yl)piperazin-1-yl)-4-methylpentyl)cyclohexyl)oxy)-2-methylphenyl)picolinate

A mixture of 3-(1-methyl-7-(4-(2-methyl-5-((1r,4s)-4-(2-methyl-3-(4,4,5,5-tetramethyl-1,3,2-dioxaborolan-2-yl)phenoxy)cyclohexyl)pentan-2-yl)piperazin-1-yl)-1H-indazol-3-yl)piperidine-2,6-dione (110 mg, 151.56 μmol, 1 equiv.), tert-butyl 6-(8-(benzo[d]thiazol-2-ylcarbamoyl)-3,4-dihydroisoquinolin-2(1H)-yl)-3-bromopicolinate (102.85 mg, 181.88 μmol, 1.2 equiv.) in dioxane (2 mL) and H₂O (0.2 mL) was added KF (26.42 mg, 454.69 μmol, 10.65 μL, 3 equiv.) and Ad₂nBuP Pd G₃ (cataCXium® A Pd G₃) (22.08 mg, 30.31 μmol, 0.2 equiv.). After addition, the mixture was degassed and purged with N₂ three times. The mixture was stirred at 100° C. for 2 hours under N₂ atmosphere. The reaction mixture was filtered and concentrated under reduced pressure to give a residue. The residue was purified by flash silica gel chromatography (0˜40% ethyl acetate/petroleum ether) to give tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-7-yl)piperazin-1-yl)-4-methylpentyl)cyclohexyl)oxy)-2-methylphenyl)picolinate (83 mg, 62.00 μmol, 40.91% yield) as a yellow solid. MS (ESI) m/z: 1084.7 [M+H]⁺.

### Step F. Procedure for Preparation of 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-7-yl)piperazin-1-yl)-4-methylpentyl)cyclohexyl)oxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-7-yl)piperazin-1-yl)-4-methylpentyl)cyclohexyl)oxy)-2-methylphenyl)picolinate (83 mg, 76.54 μmol, 1 equiv.) in DCM (1 mL) was added TFA (1.54 g, 13.46 mmol, 1 mL, 175.88 equiv.). The mixture was stirred at 25° C. for 12 hours. The reaction mixture was filtered and concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-7-yl)piperazin-1-yl)-4-methylpentyl)cyclohexyl)oxy)-2-methylphenyl)picolinic acid (39.52 mg, 37.37 μmol, 48.83% yield) as a yellow solid. MS (ESI) m/z: 1028.5 [M+H]⁺. ¹H NMR (400 MHz, DMSO-d₆) δ=13.17-12.27 (m, 2H), 10.89 (s, 1H), 8.13 (s, 1H), 8.03 (d, J=8.0 Hz, 1H), 7.79 (d, J=7.8 Hz, 1H), 7.62 (d, J=7.2 Hz, 1H), 7.52-7.28 (m, 6H), 7.13-7.00 (m, 3H), 6.94 (dd, J=8.8, 17.6 Hz, 2H), 6.61 (d, J=7.4 Hz, 1H), 4.98 (s, 2H), 4.34 (dd, J=5.6, 10.0 Hz, 1H), 4.24 (s, 3H), 4.22-4.15 (m, 1H), 3.91 (t, J=5.6 Hz, 2H), 3.02 (t, J=5.2 Hz, 2H), 2.72-2.53 (m, 6H), 2.43-2.23 (m, 2H), 2.20-2.13 (m, 1H), 2.12-2.02 (m, 2H), 1.87 (s, 3H), 1.80 (d, J=12.8 Hz, 2H), 1.65-0.80 (m, 20H).

### Example 286. Preparation of Compound 343a

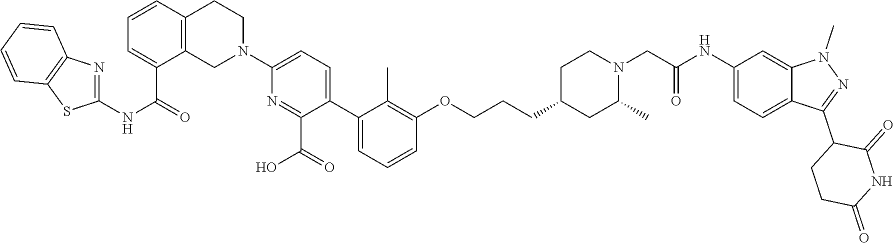 [SMILES: Cc1c(OCCC[C@@H]2CCN(CC(=O)Nc3ccc4c(C5CCC(=O)NC5=O)nn(C)c4c3)[C@H](C)C2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-((2R,4R)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinic acid

### Step A. Procedure for Preparation of (2R,4R)-4-(3-(3-bromo-2-methylphenoxy)propyl)-2-methylpiperidine

A mixture of tert-butyl (2R,4R)-4-(3-(3-bromo-2-methylphenoxy)propyl)-2-methylpiperidine-1-carboxylate (300 mg, 703.59 μmol, 1.0 equiv.) in HCl/dioxane (5 mL) was stirred at 25° C. for 1 hour. The reaction mixture was concentrated under reduced pressure to give (2R,4R)-4-(3-(3-bromo-2-methylphenoxy)propyl)-2-methylpiperidine (230 mg, 624.69 μmol, 88.79% yield, HCl) as a white solid. MS (ESI) m/z: 326.1 [M+H]⁺.

### Step B. Procedure for Preparation of ethyl 2-((2R,4R)-4-(3-(3-bromo-2-methylphenoxy)propyl)-2-methylpiperidin-1-yl)acetate

A mixture of (2R,4R)-4-(3-(3-bromo-2-methylphenoxy)propyl)-2-methylpiperidine (230 mg, 704.94 μmol, 1.0 equiv.), ethyl 2-bromoacetate (117.72 mg, 704.94 μmol, 78.02 μL, 1.0 equiv.), and K₂CO₃ (292.28 mg, 2.11 mmol, 3.0 equiv.) in DMF (5 mL) was stirred at 40° C. for 0.5 hours. The reaction mixture was partitioned between H₂O (5 mL) and ethyl acetate (10 mL×3). The organic phase was separated, washed with brine (15 mL×2), dried over Na₂SO₄, filtered and concentrated under reduced pressure to give ethyl 2-((2R,4R)-4-(3-(3-bromo-2-methylphenoxy)propyl)-2-methylpiperidin-1-yl)acetate (300 mg, crude) as a yellow solid. MS (ESI) m/z: 414.2 [M+H]⁺.

### Step C. Procedure for Preparation of tert-butyl 6-(8-(benzo[d]thiazol-2-ylcarbamoyl)-3,4-dihydroisoquinolin-2(1H)-yl)-3-(3-(3-((2R,4R)-1-(2-ethoxy-2-oxoethyl)-2-methylpiperidin-4-yl)propoxy)-2-methylphenyl)picolinate

A mixture of ethyl 2-((2R,4R)-4-(3-(3-bromo-2-methylphenoxy)propyl)-2-methylpiperidin-1-yl)acetate (300 mg, 727.52 μmol, 1.0 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (490.20 mg, 800.27 μmol, 1.1 equiv.), Ad₂nBuP Pd G₃ (105.97 mg, 145.50 μmol, 0.2 equiv.), and KF (126.80 mg, 2.18 mmol, 51.13 μL, 3.0 equiv.) in dioxane (5 mL) and H₂O (0.5 mL) was degassed and purged with N₂ three times. The mixture was stirred at 100° C. for 16 hours under N₂ atmosphere. The reaction mixture was partitioned between ethyl acetate (5 mL×3) and H₂O (5 mL). The organic phase was separated, washed with brine (8 mL×2), dried over Na₂SO₄, filtered, and concentrated under reduced pressure to give a residue. The residue was purified by flash silica gel chromatography (0˜58% ethyl acetate/petroleum ether) to give tert-butyl 6-(8-(benzo[d]thiazol-2-ylcarbamoyl)-3,4-dihydroisoquinolin-2(1H)-yl)-3-(3-(3-((2R,4R)-1-(2-ethoxy-2-oxoethyl)-2-methylpiperidin-4-yl)propoxy)-2-methylphenyl)picolinate (412 mg, 453.84 mol, 62.38% yield) as a yellow solid. MS (ESI) m/z: 818.8 [M+H]⁺.

### Step D. Procedure for Preparation of 2-((2R,4R)-4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-2-methylpiperidin-1-yl)acetic acid

A mixture of tert-butyl 6-(8-(benzo[d]thiazol-2-ylcarbamoyl)-3,4-dihydroisoquinolin-2(1H)-yl)-3-(3-(3-((2R,4R)-1-(2-ethoxy-2-oxoethyl)-2-methylpiperidin-4-yl)propoxy)-2-methylphenyl)picolinate (410 mg, 501.20 μmol, 1.0 equiv.) LiOH·H₂O (84.13 mg, 2.00 mmol, 4 equiv.) in THE (4 mL) and H₂O (1 mL) was stirred at 25° C. for 19 hours. The reaction mixture was partitioned between DCM (10 mL×3) and H₂O (10 mL). The organic phase was separated, washed with brine (20 mL×2), dried over Na₂SO₄, filtered, and concentrated under reduced pressure to give 2-((2R,4R)-4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-2-methylpiperidin-1-yl)acetic acid (398 mg, crude) as a yellow solid. MS (ESI) m/z: 790.5 [M+H]⁺. ¹H NMR (400 MHz, DMSO-d₆) δ=7.80 (dd, J=1.6, 6.8 Hz, 1H), 7.61 (d, J=7.6 Hz, 1H), 7.47-7.36 (m, 2H), 7.27-7.22 (m, 1H), 7.18-7.14 (m, 3H), 7.10 (t, J=7.6 Hz, 1H), 6.97 (t, J=7.2 Hz, 1H), 6.91 (d, J=8.8 Hz, 1H), 6.59 (d, J=7.6 Hz, 1H), 5.15 (s, 2H), 4.02-3.81 (m, 4H), 3.00-2.95 (m, 2H), 2.84 (d, J=10.8 Hz, 1H), 2.30 (s, 2H), 2.15-2.03 (m, 2H), 1.88 (s, 3H), 1.71 (d, J=6.8 Hz, 2H), 1.62 (s, 1H), 1.53 (d, J=12.0 Hz, 2H), 1.31 (s, 2H), 1.23 (d, J=2.4 Hz, 2H), 1.06 (s, 9H), 0.99 (d, J=6.0 Hz, 3H)

### Step E. Procedure for Preparation of tert-butyl 6-(8-(benzo[d]thiazol-2-ylcarbamoyl)-3,4-dihydroisoquinolin-2(1H)-yl)-3-(3-(3-((2R,4R)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinate

A mixture of 2-((2R,4R)-4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-2-methylpiperidin-1-yl)acetic acid (200 mg, 253.17 μmol, 1.0 equiv.) and EDCI (72.80 mg, 379.76 μmol, 1.5 equiv.) in pyridine (2 mL) was added 3-(6-amino-1-methyl-1H-indazol-3-yl)piperidine-2,6-dione (78.47 mg, 303.81 μmol, 1.2 equiv.). The reaction mixture was stirred at 40° C. for 3 hours. The reaction mixture was concentrated under reduced pressure to give tert-butyl 6-(8-(benzo[d]thiazol-2-ylcarbamoyl)-3,4-dihydroisoquinolin-2(1H)-yl)-3-(3-(3-((2R,4R)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinate (220 mg, crude) as a yellow solid. MS (ESI) m/z: 1030.7 [M+H]⁺

### Step F. Procedure for Preparation of 6-(8-(benzo[d]thiazol-2-ylcarbamoyl)-3,4-dihydroisoquinolin-2(1H)-yl)-3-(3-(3-((2R,4R)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinic acid

A mixture of tert-butyl 6-(8-(benzo[d]thiazol-2-ylcarbamoyl)-3,4-dihydroisoquinolin-2(1H)-yl)-3-(3-(3-((2R,4R)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinate (220 mg, 213.54 μmol, 1.0 equiv.) in TFA (3 mL) and DCM (1 mL) was stirred at 25° C. for 16 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(3-((2R,4R)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinic acid (84.23 mg, 84.58 μmol, 39.61% yield) as a yellow solid. MS (ESI) m/z: 974.7 [M+H]⁺. ¹H NMR (400 MHz, DMSO-d₆) δ=12.93
12.76 (m, 1H), 10.89 (s, 1H), 8.07-7.99 (m, 2H), 7.79 (d, J=8.0 Hz, 1H), 7.69-7.59 (m, 2H), 7.49-7.43 (m, 3H), 7.40-7.32 (m, 2H), 7.24-7.16 (m, 1H), 7.10 (t, J=7.6 Hz, 1H), 6.97 (d, J=8.8 Hz, 1H), 6.88 (d, J=8.0 Hz, 1H), 6.63 (d, J=7.6 Hz, 1H), 4.98 (s, 2H), 4.33 (dd, J=4.8, 9.6 Hz, 1H), 3.96 (t, J=5.6 Hz, 2H), 3.93 (s, 3H), 3.03 (t, J=5.6 Hz, 2H), 2.69-2.59 (m, 4H), 2.39-2.32 (m, 2H), 2.20-2.14 (m, 1H), 1.90 (s, 3H), 1.83-1.68 (m, 4H), 1.40 (d, J=2.8 Hz, 3H), 1.21-0.93 (m, 4H).

### Example 287. Preparation of Compound 343b

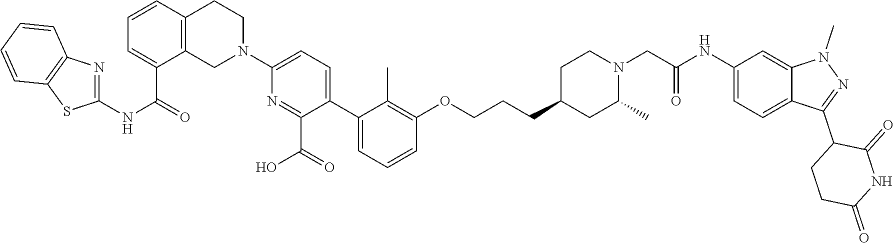 [SMILES: Cc1c(OCCC[C@H]2CCN(CC(=O)Nc3ccc4c(C5CCC(=O)NC5=O)nn(C)c4c3)[C@H](C)C2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[(2R,4S)-1-[2-[[3-(2,6-dioxo-3-piperidyl)-1-methyl-indazol-6-yl]amino]-2-oxo-ethyl]-2-methyl-4-piperidyl]propoxy]-2-methyl-phenyl]pyridine-2-carboxylic acid

### Step A. Procedure for Preparation of tert-butyl (2R,4E)-4-(3-benzyloxypropylidene)-2-methyl-piperidine-1-carboxylate

A mixture of 3-benzyloxypropyl(triphenyl)phosphonium bromide (10.14 g, 20.63 mmol, 1.1 equiv.) in THF (40 mL) was added LiHMDS (1 M, 28.13 mL, 1.5 equiv.) at 0° C. under N₂ atmosphere for 1 hour. To this mixture was added tert-butyl (2R)-2-methyl-4-oxo-piperidine-1-carboxylate (4.0 g, 18.76 mmol, 1.0 equiv.) at 0° C. under N₂ atmosphere. The mixture was stirred at 25° C. for 11 hours under N₂ atmosphere. The reaction mixture was quenched by the addition saturated NH₄Cl (100 mL) and then extracted with ethyl acetate (100 mL×3). The combined organic layers were washed with brine (90 mL×3), dried over Na₂SO₄, filtered, and concentrated under reduced pressure to give a residue. The residue was purified by column chromatography (petroleum ether/ethyl acetate=100/1 to 50/1) to give tert-butyl (2R,4E)-4-(3-benzyloxypropylidene)-2-methyl-piperidine-1-carboxylate (3.6 g, 7.92 mmol, 42.23% yield) as a yellow oil. MS (ESI) m/z: 246.4 [M+H]⁺.

### Step B. Procedure for Preparation of tert-butyl (2R)-4-(3-hydroxypropyl)-2-methyl-piperidine-1-carboxylate

To a solution of tert-butyl (2R,4E)-4-(3-benzyloxypropylidene)-2-methyl-piperidine-1-carboxylate (3.6 g, 10.42 mmol, 1.0 equiv.) in EtOH (50 mL) was added Pd/C (1.11 g, 1.04 mmol, 10% purity, 0.1 equiv.) and Pd(OH)₂ (1.10 g, 1.56 mmol, 20% purity, 0.15 equiv.) under N₂ atmosphere. The suspension was degassed and purged with H₂ three times. The mixture was stirred under H₂ (15 Psi) at 40° C. for 12 hours. The reaction mixture was filtered and concentrated under reduced pressure to give tert-butyl (2R)-4-(3-hydroxypropyl)-2-methyl-piperidine-1-carboxylate (2.4 g, 9.33 mmol, 89.49% yield) as a yellow oil. ¹H NMR (400 MHz, DMSO-d₆) δ=4.37-4.31 (m, 2H), 3.81-3.74 (m, 1H), 2.81-2.68 (m, 1H), 1.56-1.50 (m, 3H), 1.44-1.37 (m, 12H), 1.30-1.16 (m, 4H), 1.12-1.05 (m, 3H)

### Step C. Procedure for Preparation of tert-butyl (2R)-4-[3-(3-bromo-2-methyl-phenoxy)propyl]-2-methyl-piperidine-1-carboxylate

To a solution of tert-butyl (2R)-4-(3-hydroxypropyl)-2-methyl-piperidine-1-carboxylate (2.4 g, 9.33 mmol, 1.0 equiv.) in toluene (25 mL) was added 2-(tributyl-phosphanylidene)acetonitrile (3.38 g, 13.99 mmol, 1.5 equiv.) and 3-bromo-2-methyl-phenol (2.09 g, 11.19 mmol, 1.2 equiv.). The mixture was stirred at 120° C. for 12 hours. The reaction mixture was concentrated under reduced pressure to give a residue. The residue was purified by column chromatography (petroleum ether/ethyl acetate=100/1 to 10/1) and reverse-phase HPLC to give tert-butyl (2R)-4-[3-(3-bromo-2-methyl-phenoxy)propyl]-2-methyl-piperidine-1-carboxylate (1.7 g, 3.93 mmol, 42.11% yield) as a yellow oil. MS (ESI) m/z: 326.0 [M+H]⁺. ¹H NMR (400 MHz, CDCl₃) δ=7.15 (d, J=8.0 Hz, 1H), 7.00 (t, J=8.0 Hz, 1H), 6.76 (d, J=8.0 Hz, 1H), 3.94 (t, J=6.4 Hz, 3H), 2.88-2.82 (m, 1H), 2.32 (s, 3H), 1.85-1.81 (m, 2H), 1.60 (s, 4H), 1.47 (s, 9H), 1.42-1.35 (m, 2H), 1.34-1.27 (m, 1H), 1.18 (d, J=6.4 Hz, 1H), 1.14 (d, J=7.2 Hz, 3H)

### Step D. Procedure for Preparation of tert-butyl (2R,4S)-4-[3-(3-bromo-2-methyl-phenoxy)propyl]-2-methyl-piperidine-1-carboxylate and tert-butyl (2R,4R)-4-[3-(3-bromo-2-methyl-phenoxy)propyl]-2-methyl-piperidine-1-carboxylate

The stereoisomers of tert-butyl (2R)-4-[3-(3-bromo-2-methyl-phenoxy)propyl]-2-methyl-piperidine-1-carboxylate were separated by prep-HPLC (column: DAICEL CHIRALCEL OD(250 mm×30 mm, 10 um); mobile phase: [CO₂-i-PrOH(0.1% NH₃H₂O)]; B %:20%, isocratic elution mode) to give tert-butyl (2R,4S)-4-[3-(3-bromo-2-methyl-phenoxy)propyl]-2-methyl-piperidine-1-carboxylate (1 g, 2.35 mmol, 58.82% yield) as a yellow oil and tert-butyl (2R,4R)-4-[3-(3-bromo-2-methyl-phenoxy)propyl]-2-methyl-piperidine-1-carboxylate (300 mg, 703.59 mol, 17.65% yield) as a yellow oil.

¹H NMR (400 MHz, CDCl₃) δ=7.07 (d, J=8.0 Hz, 1H), 6.91 (t, J=8.0 Hz, 1H), 6.68 (d, J=8.0 Hz, 1H), 4.35 (s, 1H), 3.93-3.80 (m, 3H), 2.82-2.72 (m, 1H), 2.24 (s, 3H), 1.78-1.70 (m, 2H), 1.65-1.56 (m, 2H), 1.49 (d, J=13.8 Hz, 1H), 1.39 (s, 9H), 1.34-1.27 (m, 2H), 1.27-1.19 (m, 1H), 1.06 (d, J=7.2 Hz, 3H), 1.03-0.95 (m, 1H).

¹H NMR (400 MHz, CDCl₃) δ=7.07 (d, J=8.0 Hz, 1H), 6.91 (t, J=8.0 Hz, 1H), 6.68 (d, J=8.0 Hz, 1H), 3.86 (t, J=6.4 Hz, 2H), 3.84-3.78 (m, 1H), 3.71-3.60 (m, 1H), 3.05-2.92 (m, 1H), 2.24 (s, 3H), 1.90-1.82 (m, 1H), 1.76-1.69 (m, 3H), 1.55-1.49 (m, 1H), 1.45-1.40 (m, 2H), 1.39 (s, 9H), 1.13-1.07 (m, 5H).

### Step E. Procedure for Preparation of (2R,4S)-4-[3-(3-bromo-2-methyl-phenoxy)propyl]-2-methyl-piperidine

To a solution of tert-butyl (2R,4S)-4-[3-(3-bromo-2-methyl-phenoxy)propyl]-2-methyl-piperidine-1-carboxylate (400.00 mg, 938.12 μmol, 1.0 equiv.) in DCM (2 mL) was added HCl/dioxane (4 M, 4 mL, 17.06 equiv.). The mixture was stirred at 25° C. for 30 min. The reaction mixture was concentrated under reduced pressure to give (2R,4S)-4-[3-(3-bromo-2-methyl-phenoxy)propyl]-2-methyl-piperidine (300 mg, 919.48 μmol, 98.01% yield) as a white solid. MS (ESI) m/z: 326.1 [M+H]⁺.

### Step F. Procedure for Preparation of ethyl 2-[(2R,4S)-4-[3-(3-bromo-2-methyl-phenoxy)propyl]-2-methyl-1-piperidyl]acetate

To a solution of (2R,4S)-4-[3-(3-bromo-2-methyl-phenoxy)propyl]-2-methyl-piperidine (280.00 mg, 858.18 μmol, 1.0 equiv.) in acetone (3 mL) was added TEA (260.52 mg, 2.57 mmol, 358.34 μL, 3.0 equiv.) and ethyl 2-bromoacetate (143.32 mg, 858.18 μmol, 94.97 μL, 1.0 equiv.). The mixture was stirred at 25° C. for 12 hours. The reaction mixture was concentrated under reduced pressure to give a residue. The residue was purified by column chromatography (petroleum ether/ethyl acetate=3/1 to 1/1) to give ethyl 2-[(2R,4S)-4-[3-(3-bromo-2-methyl-phenoxy)propyl]-2-methyl-1-piperidyl]acetate (260 mg, 624.21 μmol, 72.74% yield) as a yellow oil. MS (ESI) m/z: 412.3 [M+H]⁺. ¹H NMR (400 MHz, CDCl₃) δ=7.14 (d, J=8.0 Hz, 1H), 6.99 (t, J=8.0 Hz, 1H), 6.76 (d, J=8.0 Hz, 1H), 4.24-4.14 (m, 2H), 3.94 (t, J=6.4 Hz, 2H), 3.40-3.24 (m, 2H), 3.11 (s, 1H), 2.74 (d, J=3.6 Hz, 1H), 2.63-2.53 (m, 1H), 2.32 (s, 3H), 1.85-1.78 (m, 2H), 1.67-1.61 (m, 2H), 1.59 (d, J=4.0 Hz, 2H), 1.49-1.40 (m, 3H), 1.28 (t, J=7.2 Hz, 3H), 1.03 (d, J=6.4 Hz, 3H)

### Step G. Procedure for Preparation of tert-butyl 6-[8-(1,3-benzothiazol-2-ylcarbamoyl)-3,4-dihydro-1H-isoquinolin-2-yl]-3-[3-[3-[(2R,4S)-1-(2-ethoxy-2-oxo-ethyl)-2-methyl-4-piperidyl]propoxy]-2-methyl-phenyl]pyridine-2-carboxylate

To a solution of ethyl 2-[(2R,4S)-4-[3-(3-bromo-2-methyl-phenoxy)propyl]-2-methyl-1-piperidyl]acetate (130.00 mg, 315.26 μmol, 1.0 equiv.) in dioxane (4 mL) and H₂O (1 mL) was added KF (54.95 mg, 945.78 μmol, 22.16 μL, 3.0 equiv.), [2-(2-aminophenyl)phenyl]palladium(1+); bis(1-adamantyl)-butyl-phosphane; methanesulfonate (22.96 mg, 31.53 μmol, 0.1 equiv.), and tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (212.42 mg, 346.78 μmol, 1.1 equiv.). The mixture was stirred at 100° C. for 1 hour. The reaction mixture was then concentrated under reduced pressure to give a residue. The residue was purified by column chromatography (petroleum ether/ethyl acetate=5/1 to 1/1) to give tert-butyl 6-[8-(1,3-benzothiazol-2-ylcarbamoyl)-3,4-dihydro-1H-isoquinolin-2-yl]-3-[3-[3-[(2R,4S)-1-(2-ethoxy-2-oxo-ethyl)-2-methyl-4-piperidyl]propoxy]-2-methyl-phenyl]pyridine-2-carboxylate (250 mg, 287.27 μmol, 91.12% yield) as a yellow oil. MS (ESI) m/z: 818.7 [M+H]⁺.

### Step H. Procedure for Preparation of 2-[(2R,4S)-4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-2-methyl-1-piperidyl]acetic acid

To a solution of tert-butyl 6-[8-(1,3-benzothiazol-2-ylcarbamoyl)-3,4-dihydro-1H-isoquinolin-2-yl]-3-[3-[3-[(2R,4S)-1-(2-ethoxy-2-oxo-ethyl)-2-methyl-4-piperidyl]propoxy]-2-methyl-phenyl]pyridine-2-carboxylate (240.00 mg, 293.39 μmol, 1.0 equiv.) in THE (4 mL) and H₂O (1 mL) was added LiOH·H₂O (36.93 mg, 880.16 μmol, 3.0 equiv.). The mixture was stirred at 40° C. for 1 hour. The reaction mixture was acidified to pH=5 with 1M HCl and extracted with DCM (30 mL×3). The combined organic layers were washed with brine (30 mL×1), dried over Na₂SO₄, filtered, and concentrated under reduced pressure to give 2-[(2R,4S)-4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-2-methyl-1-piperidyl]acetic acid (220 mg, 278.49 μmol, 94.92% yield) as a yellow solid. MS (ESI) m/z: 790.4 [M+H]⁺.

### Step I. Procedure for preparation of tert-butyl 6-[8-(1,3-benzothiazol-2-ylcarbamoyl)-3,4-dihydro-1H-isoquinolin-2-yl]-3-[3-[3-[(2R,4S)-1-[2-[[3-(2,6-dioxo-3-piperidyl)-1-methyl-indazol-6-yl]amino]-2-oxo-ethyl]-2-methyl-4-piperidyl]propoxy]-2-methyl-phenyl]pyridine-2-carboxylate

To a solution of 2-[(2R,4S)-4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-2-methyl-1-piperidyl]acetic acid (200 mg, 253.17 μmol, 1 equiv.) in pyridine (2 mL) was added EDCI (72.80 mg, 379.76 μmol, 1.5 equiv.) and 3-(6-amino-1-methyl-indazol-3-yl)piperidine-2,6-dione (71.93 mg, 278.49 μmol, 1.2 equiv.). The mixture was stirred at 25° C. for 1 hour. The reaction was diluted with water (300 mL) and extracted with DCM (100 mL×2). The combined organic layers were washed with brine (100 mL×1), dried over Na₂SO₄, filtered, and concentrated under reduced pressure to give tert-butyl 6-[8-(1,3-benzothiazol-2-ylcarbamoyl)-3,4-dihydro-1H-isoquinolin-2-yl]-3-[3-[3-[(2R,4S)-1-[2-[[3-(2,6-dioxo-3-piperidyl)-1-methyl-indazol-6-yl]amino]-2-oxo-ethyl]-2-methyl-4-piperidyl]propoxy]-2-methyl-phenyl]pyridine-2-carboxylate (200 mg, 194.13 mol, 76.68% yield) as a yellow solid. MS (ESI) m/z: 1030.6 [M+H]⁺.

### Step J. Procedure for Preparation of 6-[8-(1,3-benzothiazol-2-ylcarbamoyl)-3,4-dihydro-1H-isoquinolin-2-yl]-3-[3-[3-[(2R,4S)-1-[2-[[3-(2,6-dioxo-3-piperidyl)-1-methyl-indazol-6-yl]amino]-2-oxo-ethyl]-2-methyl-4-piperidyl]propoxy]-2-methyl-phenyl]pyridine-2-carboxylic acid

To a solution of tert-butyl 6-[8-(1,3-benzothiazol-2-ylcarbamoyl)-3,4-dihydro-1H-isoquinolin-2-yl]-3-[3-[3-[(2R,4S)-1-[2-[[3-(2,6-dioxo-3-piperidyl)-1-methyl-indazol-6-yl]amino]-2-oxo-ethyl]-2-methyl-4-piperidyl]propoxy]-2-methyl-phenyl]pyridine-2-carboxylate (200 mg, 194.13 mol, 1.0 equiv.) in DCM (1 mL) was added TFA (3.07 g, 26.92 mmol, 2 mL, 138.69 equiv.). The mixture was stirred at 25° C. for 12 hours. The reaction mixture was then concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[3-[3-[(2R,4S)-1-[2-[[3-(2,6-dioxo-3-piperidyl)-1-methyl-indazol-6-yl]amino]-2-oxo-ethyl]-2-methyl-4-piperidyl]propoxy]-2-methyl-phenyl]pyridine-2-carboxylic acid (37.85 mg, 36.17 μmol, 18.63% yield) as a white solid.

MS (ESI) m/z: 974.6 [M+H]⁺. ¹H NMR (400 MHz, DMSO-d₆) δ=13.00-12.54 (m, 1H), 10.86 (s, 1H), 10.01-9.89 (m, 1H), 8.06-7.99 (m, 2H), 7.78 (d, J=8.0 Hz, 1H), 7.66-7.60 (m, 2H), 7.49-7.42 (m, 3H), 7.40-7.31 (m, 2H), 7.20 (d, J=8.8 Hz, 1H), 7.09 (t, J=8.0 Hz, 1H), 6.96 (d, J=8.8 Hz, 1H), 6.88 (d, J=8.4 Hz, 1H), 6.63 (d, J=7.6 Hz, 1H), 4.98 (s, 2H), 4.36-4.29 (m, 1H), 3.96 (t, J=6.4 Hz, 2H), 3.94-3.89 (m, 5H), 3.02 (t, J=5.6 Hz, 2H), 2.76-2.52 (m, 7H), 2.37-2.30 (m, 1H), 2.21-2.14 (m, 1H), 1.90 (s, 3H), 1.79-1.65 (m, 4H), 1.58 (s, 2H), 1.46-1.38 (m, 2H), 1.34 (d, J=8.0 Hz, 1H), 1.07 (d, J=5.2 Hz, 3H)

### Example 288. Preparation of Compound 343c

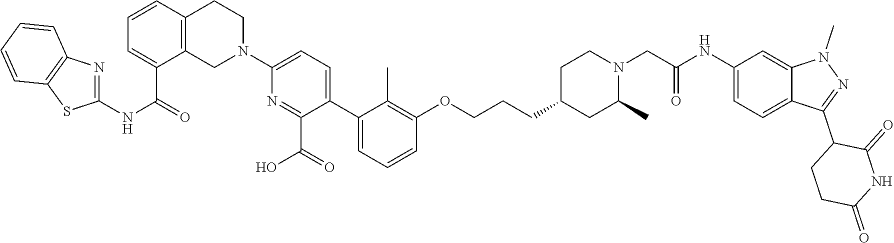 [SMILES: Cc1c(OCCC[C@@H]2CCN(CC(=O)Nc3ccc4c(C5CCC(=O)NC5=O)nn(C)c4c3)[C@@H](C)C2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(3-((2S,4R)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinic acid

### Step A. Procedure for Preparation of (2S,4R)-4-(3-(3-bromo-2-methylphenoxy)propyl)-2-methylpiperidine

To a solution of tert-butyl (2S,4R)-4-(3-(3-bromo-2-methylphenoxy)propyl)-2-methylpiperidine-1-carboxylate (320.00 mg, 750.49 μmol, 1.0 equiv.) in DCM (3 mL) was added HCl/dioxane (4 M, 187.62 μL, 1.0 equiv.). The mixture was stirred at 25° C. for 0.5 hour. The reaction mixture was concentrated under reduced pressure to give (2S,4R)-4-(3-(3-bromo-2-methylphenoxy)propyl)-2-methylpiperidine (200 mg, 612.99 μmol, 81.68% yield) as a white solid, which was used in the next step without further purification. MS (ESI) m/z: 326.0 [M+H]⁺.

### Step B. Procedure for Preparation of ethyl 2-((2S,4R)-4-(3-(3-bromo-2-methylphenoxy)propyl)-2-methylpiperidin-1-yl)acetate

To a solution of (2S,4R)-4-(3-(3-bromo-2-methylphenoxy)propyl)-2-methylpiperidine (150.00 mg, 459.74 μmol, 1.0 equiv.) in DMF (2 mL) was added K₂CO₃ (190.62 mg, 1.38 mmol, 3.0 equiv.) and ethyl 2-bromoacetate (76.78 mg, 459.74 μmol, 50.88 μL, 1.0 equiv.). The mixture was stirred at 40° C. for 1 hour. The reaction mixture was concentrated under reduced pressure to remove solvent. The residue was diluted with H₂O (100 mL) and extracted with ethyl acetate (50 mL×3). The combined organic layers were washed with DCM (50 mL), dried over Na₂SO₄, filtered, and concentrated under reduced pressure to give ethyl 2-((2S,4R)-4-(3-(3-bromo-2-methylphenoxy)propyl)-2-methylpiperidin-1-yl)acetate (150 mg, 363.76 μmol, 79.12% yield) as a yellow oil, which was used in the next step without further purification. MS (ESI) m/z: 413.1 [M+H]⁺.

### Step C. Procedure for Preparation of tert-butyl 6-(8-(benzo[d]thiazol-2-ylcarbamoyl)-3,4-dihydroisoquinolin-2(1H)-yl)-3-(3-(3-((2S,4R)-1-(2-ethoxy-2-oxoethyl)-2-methylpiperidin-4-yl)propoxy)-2-methylphenyl)picolinate

A mixture of ethyl 2-((2S,4R)-4-(3-(3-bromo-2-methylphenoxy)propyl)-2-methylpiperidin-1-yl)acetate (150.00 mg, 363.76 μmol, 1.0 equiv.), tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (245.10 mg, 400.14 μmol, 1.1 equiv.), KF (63.40 mg, 1.09 mmol, 25.56 μL, 3.0 equiv.), [2-(2-aminophenyl)phenyl]palladium(1+) bis(1-adamantyl)-butyl-phosphane methanesulfonate (26.49 mg, 36.38 μmol, 0.1 equiv.), and H₂O (0.5 mL) in dioxane (2 mL) was degassed and purged with N₂ three times. The mixture was stirred at 100° C. for 1 hour under N₂ atmosphere. The reaction mixture was concentrated under reduced pressure to remove solvent. The residue was diluted with H₂O (100 mL) and extracted with ethyl acetate (50 mL×3). The combined organic layers were washed with ethyl acetate (50 mL), dried over Na₂SO₄, filtered, and concentrated under reduced pressure to give a residue. The residue was purified by column chromatography (petroleum ether/ethyl acetate=8/1 to 2/1) to give tert-butyl 6-(8-(benzo[d]thiazol-2-ylcarbamoyl)-3,4-dihydroisoquinolin-2(1H)-yl)-3-(3-(3-((2S,4R)-1-(2-ethoxy-2-oxoethyl)-2-methylpiperidin-4-yl)propoxy)-2-methylphenyl)picolinate (290 mg, 319.06 μmol, 87.71% yield) as a yellow solid.

MS (ESI) m/z: 818.4 [M+H]⁺.

### Step D. Procedure for Preparation of 2-((2S,4R)-4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-2-methylpiperidin-1-yl)acetic acid

To a solution of tert-butyl 6-(8-(benzo[d]thiazol-2-ylcarbamoyl)-3,4-dihydroisoquinolin-2(1H)-yl)-3-(3-(3-((2S,4R)-1-(2-ethoxy-2-oxoethyl)-2-methylpiperidin-4-yl)propoxy)-2-methylphenyl)picolinate (290.00 mg, 354.51 μmol, 1.0 equiv.) in THE (3 mL) was added LiOH·H₂O (44.63 mg, 1.06 mmol, 3.0 equiv.) and H₂O (1 mL). The mixture was stirred at 25° C. for 12 hours. The reaction mixture was concentrated under reduced pressure to remove solvent. The residue was diluted with H₂O (100 mL) and extracted with DCM (50 mL×3). The combined organic layers were washed with DCM (50 mL), dried over Na₂SO₄, filtered, and concentrated under reduced pressure to give 2-((2S,4R)-4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-2-methylpiperidin-1-yl)acetic acid (260 mg, 329.12 μmol, 92.84% yield) as a yellow oil which was used in the next step without further purification. MS (ESI) m/z: 790.5 [M+H]⁺.

### Step E. Procedure for Preparation of tert-butyl 6-(8-(benzo[d]thiazol-2-ylcarbamoyl)-3,4-dihydroisoquinolin-2(1H)-yl)-3-(3-(3-((2S,4R)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinate

To a solution of 2-((2S,4R)-4-(3-(3-(6-(8-(benzo[d]thiazol-2-ylcarbamoyl)-3,4-dihydroisoquinolin-2(1H)-yl)-2-(tert-butoxycarbonyl)pyridin-3-yl)-2-methylphenoxy)propyl)-2-methylpiperidin-1-yl)acetic acid (150 mg, 189.88 μmol, 1.0 equiv.) in pyridine (2 mL) was added EDCI (47.32 mg, 246.84 μmol, 1.3 equiv.) and 3-(6-amino-1-methyl-1H-indazol-3-yl)piperidine-2,6-dione (58.85 mg, 227.85 μmol, 1.2 equiv.). The mixture was stirred at 40° C. for 1 hour. The reaction mixture was concentrated under reduced pressure to give a residue. The residue was diluted with H₂O (60 mL) and extracted with DCM (50 mL×2). The combined organic layers were washed with DCM (30 mL), dried over Na₂SO₄, filtered, and concentrated under reduced pressure to give tert-butyl 6-(8-(benzo[d]thiazol-2-ylcarbamoyl)-3,4-dihydroisoquinolin-2(1H)-yl)-3-(3-(3-((2S,4R)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinate (150 mg, 145.60 μmol, 76.68% yield) as a yellow oil, which was used in the next step without further purification. MS (ESI) m/z: 1030.8 [M+H]⁺.

### Step F. Procedure for Preparation of 6-(8-(benzo[d]thiazol-2-ylcarbamoyl)-3,4-dihydroisoquinolin-2(1H)-yl)-3-(3-(3-((2S,4R)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(3-((2S,4R)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinate (150 mg, 145.60 μmol, 1.0 equiv.) in DCM (1 mL) was added TFA (3 mL). The mixture was stirred at 40° C. for 1 hour. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(3-((2S,4R)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinic acid (61.41 mg, 57.56 mol, 39.53% yield) as a yellow solid. MS (ESI) m/z: 974.6 [M+H]⁺. ¹H NMR (400 MHz, DMSO-d₆) δ=12.95-12.79 (m, 1H), 12.73-12.45 (m, 1H), 10.89 (s, 1H), 8.14 (s, 1H), 8.06-8.01 (m, 2H), 7.79 (d, J=8.4 Hz, 1H), 7.65 (dd, J=8.4, 12.8 Hz, 2H), 7.50-7.44 (m, 3H), 7.37 (td, J=7.6, 11.4 Hz, 2H), 7.20 (d, J=8.8 Hz, 1H), 7.13-7.08 (m, 1H), 6.98 (d, J=8.8 Hz, 1H), 6.92-6.87 (m, 1H), 6.64 (d, J=7.6 Hz, 1H), 4.99 (s, 2H), 4.34 (dd, J=5.2, 9.6 Hz, 1H), 3.98 (t, J=6.0 Hz, 2H), 3.94-3.90 (m, 5H), 3.03 (t, J=5.6 Hz, 2H), 2.66-2.56 (m, 8H), 2.18 (dd, J=5.6, 13.2 Hz, 1H), 1.91 (s, 3H), 1.81-1.65 (m, 5H), 1.52-1.35 (m, 4H), 1.26-1.02 (m, 3H)

### Example 289. Preparation of Compound 343d

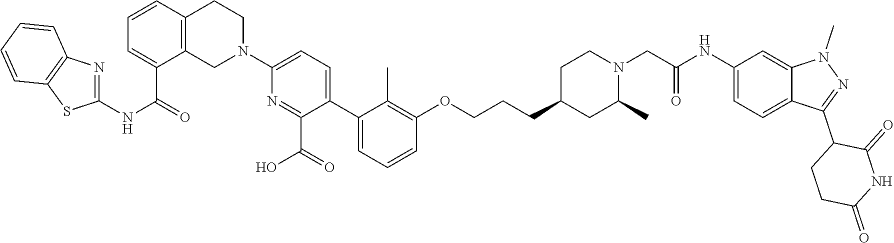 [SMILES: Cc1c(OCCC[C@H]2CCN(CC(=O)Nc3ccc4c(C5CCC(=O)NC5=O)nn(C)c4c3)[C@@H](C)C2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[(2S,4S)-1-[2-[[3-(2,6-dioxo-3-piperidyl)-1-methyl-indazol-6-yl]amino]-2-oxo-ethyl]-2-methyl-4-piperidyl]propoxy]-2-methyl-phenyl]pyridine-2-carboxylic acid

### Step A. Procedure for Preparation of tert-butyl (2S,4E)-4-(3-benzyloxypropylidene)-2-methyl-piperidine-1-carboxylate

To a solution of 3-benzyloxypropyl(triphenyl)phosphoniumbromide (11.06 g, 22.51 mmol, 1.2 equiv.) in THF (100 mL) was added LiHMDS (1 M, 37.51 mL, 2.0 equiv.) at 0° C. The mixture was stirred for 2 hours. To this solution was added tert-butyl (2S)-2-methyl-4-oxo-piperidine-1-carboxylate (4 g, 18.76 mmol, 1.0 equiv.) in THE (20 mL). The resulting mixture was stirred at 25° C. for 10 hours. The reaction mixture was quenched by addition saturated NH₄Cl (50 mL) under 0° C. The mixture was extracted with ethyl acetate (100 mL), and the combined organic layers were washed with brine (50 mL), dried over Na₂SO₄, filtered, and concentrated under reduced pressure to give a residue. The residue was purified by flash silica gel chromatography (0˜3% ethyl acetate/petroleum ether) to give tert-butyl (2S,4E)-4-(3-benzyloxypropylidene)-2-methyl-piperidine-1-carboxylate (1.44 g, 4.08 mmol, 21.78% yield) as a yellow oil. MS (ESI) m/z: 246.3 [M+H]⁺.

### Step B. Procedure for Preparation of tert-butyl (2S)-4-(3-hydroxypropyl)-2-methyl-piperidine-1-carboxylate

A mixture of tert-butyl (2S,4E)-4-(3-benzyloxypropylidene)-2-methyl-piperidine-1-carboxylate (1.44 g, 4.17 mmol, 1 equiv.), Pd/C (887.16 mg, 833.64 μmol, 10% purity, 0.2 equiv.), and Pd(OH)₂ (585.36 mg, 833.64 μmol, 20% purity, 0.2 equiv.) in MeOH (20 mL) was degassed and purged with H₂ three times. The mixture was stirred at 40° C. for 12 hours under H₂ atmosphere. The mixture was filtered and washed with MeOH (150 mL). The resulting filtrate was concentrated under reduced pressure to give a residue. The filter cake was immersed in MeOH and concentrated under vacuum to give tert-butyl (2S)-4-(3-hydroxypropyl)-2-methyl-piperidine-1-carboxylate (1 g, crude) as a yellow oil. ¹H NMR (400 MHz, DMSO-d₆) δ=4.40-4.26 (m, 1H), 3.85-3.67 (m, 1H), 3.37 (q, J=6.0 Hz, 2H), 3.32 (s, 1H), 2.91-2.63 (m, 1H), 1.67-1.42 (m, 4H), 1.39 (s, 9H), 1.32-1.22 (m, 1H), 1.20-1.03 (m, 6H), 0.95-0.78 (m, 1H)

### Step C. Procedure for Preparation of tert-butyl (2S)-4-[3-(3-bromo-2-methyl-phenoxy)propyl]-2-methyl-piperidine-1-carboxylate

A mixture of tert-butyl (2S)-4-(3-hydroxypropyl)-2-methyl-piperidine-1-carboxylate (1 g, 3.89 mmol, 1 equiv.), 3-bromo-2-methyl-phenol (872.06 mg, 4.66 mmol, 1.2 equiv.), and 2-(tributyl-phosphanylidene)acetonitrile (1.41 g, 5.83 mmol, 1.5 equiv.) in toluene (20 mL) was degassed and purged with N₂ three times. The mixture was stirred at 120° C. for 12 hours under N₂ atmosphere. The reaction mixture was concentrated under reduced pressure to give a residue. The residue was purified by flash silica gel chromatography (0˜2% ethyl acetate/petroleum ether) and SFC to give tert-butyl (2S)-4-[3-(3-bromo-2-methyl-phenoxy)propyl]-2-methyl-piperidine-1-carboxylate (1.4 g, 3.15 mmol, 81.09% yield) as a yellow oil. MS (ESI) m/z: 362.2 [M−100+H]⁺. ¹H NMR (400 MHz, DMSO-d₆) δ=7.18-7.13 (m, 1H), 7.12-7.06 (m, 1H), 6.97 (d, J=8.0 Hz, 1H), 4.38-4.19 (m, 1H), 3.98 (t, J=6.4 Hz, 2H), 3.81 (d, J=9.2 Hz, 1H), 2.92-2.70 (m, 1H), 2.24 (s, 3H), 1.79-1.60 (m, 4H), 1.59-1.49 (m, 1H), 1.39 (s, 9H), 1.36-1.27 (m, 2H), 1.24-1.15 (m, 1H), 1.13-1.04 (m, 3H), 1.01-0.63 (m, 1H)

### Step D. Procedure for Preparation of tert-butyl (2S,4S)-4-(3-(3-bromo-2-methylphenoxy)propyl)-2-methylpiperidine-1-carboxylate and tert-butyl (2S,4R)-4-(3-(3-bromo-2-methylphenoxy)propyl)-2-methylpiperidine-1-carboxylate

The tert-butyl (2S)-4-[3-(3-bromo-2-methyl-phenoxy)propyl]-2-methyl-piperidine-1-carboxylate as a stereoisomeric mixture was separated by SFC to give tert-butyl (2S,4S)-4-(3-(3-bromo-2-methylphenoxy)propyl)-2-methylpiperidine-1-carboxylate (320 mg, 750.49 μmol, 43.84% yield) as a yellow oil and tert-butyl (2S,4R)-4-(3-(3-bromo-2-methylphenoxy)propyl)-2-methylpiperidine-1-carboxylate (170 mg, 398.70 μmol, 23.29% yield) as a yellow oil. ¹H NMR (400 MHz, CDCl₃) δ=7.06 (d, J=8.0 Hz, 1H), 6.91 (t, J=8.0 Hz, 1H), 6.67 (d, J=8.4 Hz, 1H), 3.92-3.78 (m, 3H), 3.65 (dd, J=7.2, 14.0 Hz, 1H), 3.04-2.93 (m, 1H), 2.24 (s, 3H), 1.92-1.80 (m, 1H), 1.79-1.64 (m, 3H), 1.49 (s, 2H), 1.44-1.41 (m, 1H), 1.39 (s, 9H), 1.13-1.06 (m, 5H) 1H NMR (400 MHz, CDCl₃) δ=7.07 (d, J=8.0 Hz, 1H), 6.91 (t, J=8.0 Hz, 1H), 6.68 (d, J=8.0 Hz, 1H), 3.86 (t, J=6.4 Hz, 3H), 2.77 (t, J=13.2 Hz, 1H), 2.24 (s, 3H), 1.79-1.71 (m, 2H), 1.66-1.46 (m, 4H), 1.39 (s, 9H), 1.35-1.17 (m, 4H), 1.06 (d, J=6.8 Hz, 3H)

### Step E. Procedure for Preparation of (2S,4S)-4-[3-(3-bromo-2-methyl-phenoxy)propyl]-2-methyl-piperidine

A solution of tert-butyl (2S,4S)-4-(3-(3-bromo-2-methylphenoxy)propyl)-2-methylpiperidine-1-carboxylate (190 mg, 445.61 μmol, 1 equiv.) in HCl/dioxane (3 mL) and DCM (1 mL) was stirred at 25° C. for 1.5 hours. The reaction mixture was concentrated under reduced pressure to give (2S,4S)-4-[3-(3-bromo-2-methyl-phenoxy)propyl]-2-methyl-piperidine (145 mg, 444.42 μmol, 99.73% yield) as a white solid which was used in the next step without further purification. MS (ESI) m/z: 362.0 [M+H]⁺.

### Step F. Procedure for Preparation of ethyl 2-[(2S,4S)-4-[3-(3-bromo-2-methyl-phenoxy)propyl]-2-methyl-1-piperidyl]acetate

To a solution of (2S,4S)-4-[3-(3-bromo-2-methyl-phenoxy)propyl]-2-methyl-piperidine (145 mg, 444.42 μmol, 1 equiv.) and ethyl 2-bromoacetate (74.22 mg, 444.42 μmol, 49.18 μL, 1 equiv.) in DMF (2 mL) was added K₂CO₃ (184.27 mg, 1.33 mmol, 3 equiv.). The mixture was stirred at 40° C. for 1.5 hours. The reaction mixture was partitioned between ethyl acetate (20 mL) and water (10 mL). The organic phase was separated, washed with brine (10 mL), dried over Na₂SO₄, filtered and concentrated under reduced pressure to give ethyl 2-[(2S,4S)-4-[3-(3-bromo-2-methyl-phenoxy)propyl]-2-methyl-1-piperidyl]acetate (190 mg, crude) as a yellow oil. MS (ESI) m/z: 412.1 [M+H]⁺.

### Step G. Procedure for Preparation of tert-butyl 6-[8-(1,3-benzothiazol-2-ylcarbamoyl)-3,4-dihydro-1H-isoquinolin-2-yl]-3-[3-[3-[(2S,4S)-1-(2-ethoxy-2-oxo-ethyl)-2-methyl-4-piperidyl]propoxy]-2-methyl-phenyl]pyridine-2-carboxylate

A mixture of ethyl 2-[(2S,4S)-4-[3-(3-bromo-2-methyl-phenoxy)propyl]-2-methyl-1-piperidyl]acetate (150 mg, 363.76 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (267.38 mg, 436.51 μmol, 1.2 equiv.), KF (63.40 mg, 1.09 mmol, 25.56 μL, 3.0 equiv.), and Ad2nBuP Pd G₃ (cataCXium® A Pd G₃) (52.98 mg, 72.75 μmol, 0.2 equiv.) in dioxane (5 mL) and H₂O (0.5 mL) was degassed and purged with N₂ three times. The mixture was stirred at 100° C. for 1 hour under N₂ atmosphere. The reaction mixture was partitioned between ethyl acetate (30 mL) and water (20 mL). The organic phase was separated, washed with brine (20 mL), dried over Na₂SO₄, filtered, and concentrated under reduced pressure to give a residue. The residue was purified by flash silica gel chromatography (0-45% ethyl acetate/petroleum ether) to give tert-butyl 6-[8-(1,3-benzothiazol-2-ylcarbamoyl)-3,4-dihydro-1H-isoquinolin-2-yl]-3-[3-[3-[(2S,4S)-1-(2-ethoxy-2-oxo-ethyl)-2-methyl-4-piperidyl]propoxy]-2-methyl-phenyl]pyridine-2-carboxylate (260 mg, 295.59 μmol, 81.26% yield) as a yellow oil. MS (ESI) m/z: 818.3 [M+H]⁺.

### Step H. Procedure for Preparation of 2-[(2S,4S)-4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-2-methyl-1-piperidyl]acetic acid

To a solution of tert-butyl 6-[8-(1,3-benzothiazol-2-ylcarbamoyl)-3,4-dihydro-1H-isoquinolin-2-yl]-3-[3-[3-[(2S,4S)-1-(2-ethoxy-2-oxo-ethyl)-2-methyl-4-piperidyl]propoxy]-2-methyl-phenyl]pyridine-2-carboxylate (130 mg, 158.92 μmol, 1.0 equiv.) in THE (3 mL) and H₂O (1 mL) was added LiOH·H₂O (20.01 mg, 476.75 μmol, 3.0 equiv.). The mixture was stirred at 40° C. for 6 hours. The reaction mixture was acidified to pH=3-4 with citric acid and partitioned between DCM (20 mL) and water (15 mL). The organic phase was separated, washed with brine (15 mL), dried over Na₂SO₄, filtered, and concentrated under reduced pressure to give 2-[(2S,4S)-4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-2-methyl-1-piperidyl]acetic acid (100 mg, crude) as a yellow solid. MS (ESI) m/z: 790.4 [M+H]⁺.

### Step I. Procedure for preparation of tert-butyl 6-[8-(1,3-benzothiazol-2-ylcarbamoyl)-3,4-dihydro-1H-isoquinolin-2-yl]-3-[3-[3-[(2S,4S)-1-[2-[[3-(2,6-dioxo-3-piperidyl)-1-methyl-indazol-6-yl]amino]-2-oxo-ethyl]-2-methyl-4-piperidyl]propoxy]-2-methyl-phenyl]pyridine-2-carboxylate

To a solution of 2-[(2S,4S)-4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-2-methyl-1-piperidyl]acetic acid (100 mg, 126.59 μmol, 1 equiv.) and 3-(6-amino-1-methyl-indazol-3-yl)piperidine-2,6-dione (39.23 mg, 151.90 μmol, 1.2 equiv.) in pyridine (1 mL) was added EDCI (36.40 mg, 189.88 μmol, 1.5 equiv.). The mixture was stirred at 40° C. for 1 hour. The reaction mixture was partitioned between DCM (30 mL) and water (20 mL). The organic phase was separated, washed with brine (20 mL), dried over Na₂SO₄, filtered, and concentrated under reduced pressure to give tert-butyl 6-[8-(1,3-benzothiazol-2-ylcarbamoyl)-3,4-dihydro-1H-isoquinolin-2-yl]-3-[3-[3-[(2S,4S)-1-[2-[[3-(2,6-dioxo-3-piperidyl)-1-methyl-indazol-6-yl]amino]-2-oxo-ethyl]-2-methyl-4-piperidyl]propoxy]-2-methyl-phenyl]pyridine-2-carboxylate (100 mg, crude) as a yellow oil. MS (ESI) m/z: 516.1 [M/2+H]⁺.

### Step J. Procedure for Preparation of 6-[8-(1,3-benzothiazol-2-ylcarbamoyl)-3,4-dihydro-1H-isoquinolin-2-yl]-3-[3-[3-[(2S,4S)-1-[2-[[3-(2,6-dioxo-3-piperidyl)-1-methyl-indazol-6-yl]amino]-2-oxo-ethyl]-2-methyl-4-piperidyl]propoxy]-2-methyl-phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[3-[(2S,4S)-1-[2-[[3-(2,6-dioxo-3-piperidyl)-1-methyl-indazol-6-yl]amino]-2-oxo-ethyl]-2-methyl-4-piperidyl]propoxy]-2-methyl-phenyl]pyridine-2-carboxylate (130 mg, 126.18 μmol, 1 equiv.) in TFA (3 mL) and DCM (1 mL) was stirred at 25° C. for 12 hours. The reaction mixture was concentrated under reduced pressure to give a residue. The residue was purified by reverse-phase HPLC to give 6-[8-(1,3-benzothiazol-2-ylcarbamoyl)-3,4-dihydro-1H-isoquinolin-2-yl]-3-[3-[3-[(2S,4S)-1-[2-[[3-(2,6-dioxo-3-piperidyl)-1-methyl-indazol-6-yl]amino]-2-oxo-ethyl]-2-methyl-4-piperidyl]propoxy]-2-methyl-phenyl]pyridine-2-carboxylic acid (40.67 mg, 37.77 μmol, 29.93% yield) as a yellow solid. MS (ESI) m/z: 487.9 [M12+H]⁺. ¹H NMR (400 MHz, DMSO-d₆) δ=12.96-12.51 (m, 2H), 10.88 (s, 1H), 9.89-9.71 (m, 1H), 8.18-8.09 (m, 1H), 8.03 (s, 2H), 7.78 (s, 1H), 7.63 (d, J=8.0 Hz, 2H), 7.45 (d, J=8.0 Hz, 3H), 7.36 (d, J=11.2 Hz, 2H), 7.26-7.19 (m, 1H), 7.13-7.06 (m, 1H), 6.97 (d, J=8.8 Hz, 1H), 6.88 (d, J=8.4 Hz, 1H), 6.63 (d, J=7.6 Hz, 1H), 4.98 (s, 2H), 4.37-4.27 (m, 1H), 3.98-3.94 (m, 2H), 3.92 (s, 5H), 3.19-3.06 (m, 1H), 3.03 (d, J=5.6 Hz, 2H), 2.99-2.87 (m, 1H), 2.68-2.60 (m, 2H), 2.41-2.30 (m, 3H), 2.20-2.14 (m, 1H), 1.90 (s, 3H), 1.83-1.60 (m, 5H), 1.39 (s, 3H), 1.30-1.21 (m, 1H), 1.06 (d, J=4.0 Hz, 4H)

### Example 290. Preparation of Compound 344a

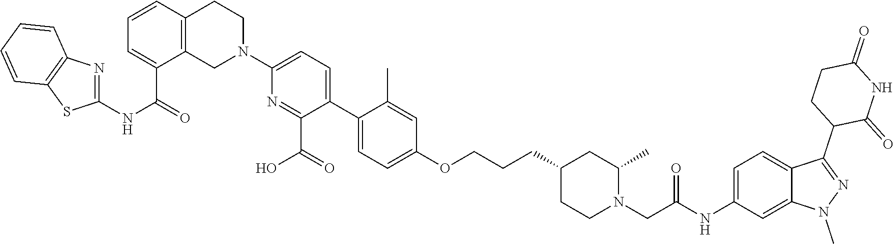 [SMILES: Cc1cc(OCCC[C@H]2CCN(CC(=O)Nc3ccc4c(C5CCC(=O)NC5=O)nn(C)c4c3)[C@@H](C)C2)ccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[4-[3-[(2S,4S)-1-[2-[[3-(2,6-dioxo-3-piperidyl)-1-methyl-indazol-6-yl]amino]-2-oxo-ethyl]-2-methyl-4-piperidyl]propoxy]-2-methyl-phenyl]pyridine-2-carboxylic acid

### Step A. Procedure for Preparation of (2S,4S)-4-[3-(4-bromo-3-methyl-phenoxy)propyl]-2-methyl-piperidine

A mixture of tert-butyl (2S,4S)-4-[3-(4-bromo-3-methyl-phenoxy)propyl]-2-methyl-piperidine-1-carboxylate (230 mg, 539.42 μmol, 1.0 equiv.) in HCl/dioxane (10 mL) was degassed and purged with N₂ three times. The mixture was stirred at 25° C. for 2 hours under N₂ atmosphere. The combined organic layers were filtered and concentrated under reduced pressure to give (2S,4S)-4-[3-(4-bromo-3-methyl-phenoxy)propyl]-2-methyl-piperidine (55 mg, crude) as a white solid, which was used in the next step without further purification. MS (ESI) m/z: 327.8 [M+H]⁺

### Step B. Procedure for Preparation of ethyl 2-[(2S,4S)-4-[3-(4-bromo-3-methyl-phenoxy)propyl]-2-methyl-1-piperidyl]acetate

A mixture of (2S,4S)-4-[3-(4-bromo-3-methyl-phenoxy)propyl]-2-methyl-piperidine (230 mg, 704.94 μmol, 1.0 equiv.), ethyl 2-bromoacetate (123.61 mg, 740.18 μmol, 81.92 μL, 1.05 equiv.), and TEA (214.00 mg, 2.11 mmol, 294.36 μL, 3.0 equiv.) in acetone (5 mL) was degassed and purged with N₂ three times. The mixture was stirred at 30° C. for 10 hours under N₂ atmosphere. The combined organic layers were filtered and concentrated under reduced pressure to give a residue. The residue was purified by flash silica gel chromatography (0˜100% ethyl acetate/petroleum ether) to give ethyl 2-[(2S,4S)-4-[3-(4-bromo-3-methyl-phenoxy)propyl]-2-methyl-1-piperidyl]acetate (230 mg, 557.77 μmol, 79.12% yield) as a white solid. MS (ESI) m/z: 412.2 [M+H]⁺

### Step C. Procedure for Preparation of tert-butyl 6-[8-(1,3-benzothiazol-2-ylcarbamoyl)-3,4-dihydro-1H-isoquinolin-2-yl]-3-[4-[3-[(2S,4S)-1-(2-ethoxy-2-oxo-ethyl)-2-methyl-4-piperidyl]propoxy]-2-methyl-phenyl]pyridine-2-carboxylate

A mixture of tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (392.16 mg, 640.22 μmol, 1.2 equiv.), ethyl 2-[(2S,4S)-4-[3-(4-bromo-3-methyl-phenoxy)propyl]-2-methyl-1-piperidyl]acetate (250 mg, 606.27 μmol, 1 equiv.), Ad2nBuP Pd G3 (77.71 mg, 106.70 μmol, 0.2 equiv.), and K₂CO₃ (1.5 M, 1.07 mL, 3 equiv.) in dioxane (5 mL) was degassed and purged with N₂ three times. The mixture was stirred at 100° C. for 1 hour under N₂ atmosphere in a microwave reactor. The reaction mixture was filtered and concentrated under reduced pressure to give a residue. The residue was purified by flash silica gel chromatography (0˜55% ethyl acetate/petroleum ether) to give tert-butyl 6-[8-(1,3-benzothiazol-2-ylcarbamoyl)-3,4-dihydro-1H-isoquinolin-2-yl]-3-[4-[3-[(2S,4S)-1-(2-ethoxy-2-oxo-ethyl)-2-methyl-4-piperidyl]propoxy]-2-methyl-phenyl]pyridine-2-carboxylate (360 mg, 440.08 μmol, 82.49% yield) as a yellow solid.

MS (ESI) m/z: 818.7 [M+H]⁺.

### Step D. Procedure for Preparation of 2-[(2S,4S)-4-[3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]propyl]-2-methyl-1-piperidyl]acetic acid

A mixture of tert-butyl 6-[8-(1,3-benzothiazol-2-ylcarbamoyl)-3,4-dihydro-1H-isoquinolin-2-yl]-3-[4-[3-[(2S,4S)-1-(2-ethoxy-2-oxo-ethyl)-2-methyl-4-piperidyl]propoxy]-2-methyl-phenyl]pyridine-2-carboxylate (360 mg, 440.08 μmol, 1 equiv.), and LiOH·H₂O (55.40 mg, 1.32 mmol, 3.0 equiv.) in H₂O (1.5 mL) and THF (3 mL) was degassed and purged with N₂ three times. The mixture was stirred at 25° C. for 10 hours under N₂ atmosphere. The combined organic layers were filtered and concentrated under reduced pressure to give 2-[(2S,4S)-4-[3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]propyl]-2-methyl-1-piperidyl]acetic acid (350 mg, crude) as a yellow solid, which was used in the next step without further purification. MS (ESI) m/z: 790.6 [M+H]⁺.

¹H NMR (400 MHz, DMSO-d₆) δ=13.03-12.67 (m, 1H), 8.01 (d, J=8.0 Hz, 1H), 7.80-7.74 (m, 1H), 7.60 (d, J=7.6 Hz, 1H), 7.49-7.30 (m, 6H), 7.02-6.89 (m, 1H), 6.86 (d, J=8.4 Hz, 1H), 6.81 (d, J=2.4 Hz, 1H), 6.71 (dd, J=2.4, 8.4 Hz, 1H), 4.96 (s, 2H), 4.05-3.92 (m, 4H), 3.89-3.79 (m, 4H), 3.60 (dd, J=2.8, 4.0, 6.4 Hz, 2H), 3.48 (d, J=12.4 Hz, 2H), 3.33 (dd, J=6.4, 9.2 Hz, 2H), 3.13-2.96 (m, 4H), 2.35-2.18 (m, 2H), 2.00 (s, 3H), 1.85-1.62 (m, 9H), 1.23 (d, J=6.4 Hz, 3H)

### Step E. Procedure for Preparation of tert-butyl 6-(8-(benzo[d]thiazol-2-ylcarbamoyl)-3,4-dihydroisoquinolin-2(1H)-yl)-3-(4-(3-((2S,4S)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinate

A mixture of 2-[(2S,4S)-4-[3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]propyl]-2-methyl-1-piperidyl]acetic acid (60 mg, 78.54 μmol, 1.0 equiv.), EDCI (22.58 mg, 117.81 μmol, 1.5 equiv.), and 3-(6-amino-1-methyl-1H-indazol-3-yl)piperidine-2,6-dione (24.34 mg, 94.25 μmol, 1.2 equiv.) in pyridine (2 mL) was degassed and purged with N₂ three times. The mixture was stirred at 25° C. for 2 hours under N₂ atmosphere. The combined organic layers were filtered and concentrated under reduced pressure to give a residue. The residue was purified by prep-TLC (SiO₂, DCM:MeOH=10:1) to give tert-butyl 6-(8-(benzo[d]thiazol-2-ylcarbamoyl)-3,4-dihydroisoquinolin-2(1H)-yl)-3-(4-(3-((2S,4S)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinate (30 mg, 29.87 μmol, 38.04% yield) as a yellow solid. MS (ESI) m/z: 1030.6 [M+H]⁺.

### Step F. Procedure for Preparation of 6-[8-(1,3-benzothiazol-2-ylcarbamoyl)-3,4-dihydro-1H-isoquinolin-2-yl]-3-[4-[3-[(2S,4S)-1-[2-[[3-(2,6-dioxo-3-piperidyl)-1-methyl-indazol-6-yl]amino]-2-oxo-ethyl]-2-methyl-4-piperidyl]propoxy]-2-methyl-phenyl]pyridine-2-carboxylic acid

A mixture of tert-butyl 6-(8-(benzo[d]thiazol-2-ylcarbamoyl)-3,4-dihydroisoquinolin-2(1H)-yl)-3-(4-(3-((2S,4S)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinate (120 mg, 116.48 μmol, 1.0 equiv.) in TFA (1 mL) and DCM (1 mL) was degassed and purged with N₂ three times. The mixture was stirred at 25° C. for 16 hours under N₂ atmosphere. The combined organic layers were filtered and concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[4-[3-[(2S,4S)-1-[2-[[3-(2,6-dioxo-3-piperidyl)-1-methyl-indazol-6-yl]amino]-2-oxo-ethyl]-2-methyl-4-piperidyl]propoxy]-2-methyl-phenyl]pyridine-2-carboxylic acid (46.66 mg, 47.44 μmol, 40.73% yield) as a white solid. MS (ESI) m/z: 974.5 [M+H]⁺. ¹H NMR (400 MHz, DMSO-d₆) δ=13.02-12.29 (m, 2H), 10.88 (s, 1H), 9.89-9.73 (m, 1H), 8.06-7.99 (m, 2H), 7.79 (d, J=8.0 Hz, 1H), 7.66-7.59 (m, 2H), 7.51-7.42 (m, 3H), 7.40-7.32 (m, 2H), 7.23 (dd, J=1.2, 8.8 Hz, 1H), 6.93 (dd, J=8.8, 14.8 Hz, 2H), 6.79 (d, J=2.4 Hz, 1H), 6.71 (dd, J=2.4, 8.4 Hz, 1H), 4.97 (s, 2H), 4.33 (dd, J=5.2, 9.6 Hz, 1H), 3.97-3.89 (m, 7H), 3.10-2.89 (m, 4H), 2.72-2.54 (m, 3H), 2.35 (dt, J=4.8, 9.2 Hz, 2H), 2.21-2.13 (m, 1H), 2.03 (s, 3H), 1.76-1.62 (m, 4H), 1.49-1.17 (m, 5H), 1.11-0.99 (m, 4H)

### Example 291. Preparation of Compound 344b

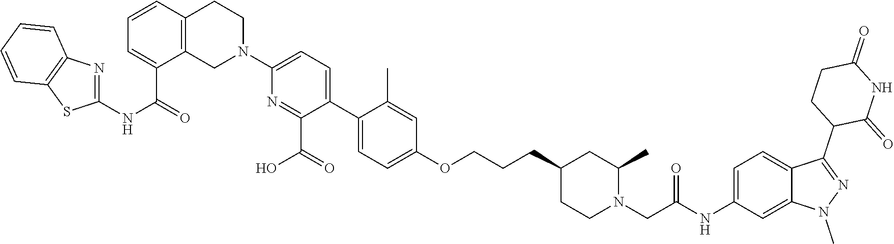 [SMILES: Cc1cc(OCCC[C@@H]2CCN(CC(=O)Nc3ccc4c(C5CCC(=O)NC5=O)nn(C)c4c3)[C@H](C)C2)ccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[4-[3-[(2R,4R)-1-[2-[[3-(2,6-dioxo-3-piperidyl)-1-methyl-indazol-6-yl]amino]-2-oxo-ethyl]-2-methyl-4-piperidyl]propoxy]-2-methyl-phenyl]pyridine-2-carboxylic acid

### Step A. Procedure for Preparation of (2R,4R)-4-[3-(4-bromo-3-methyl-phenoxy)propyl]-2-methyl-piperidine

A mixture of tert-butyl (2R,4R)-4-[3-(4-bromo-3-methyl-phenoxy)propyl]-2-methyl-piperidine-1-carboxylate (300.00 mg, 703.59 μmol, 1 equiv.) in DCM (5 mL) and HCl/dioxane (5 mL) was degassed and purged with N₂ three times. The mixture was stirred at 25° C. for 1 hour under N₂ atmosphere. The reaction mixture was then filtered and concentrated under reduced pressure to give (2R,4R)-4-[3-(4-bromo-3-methyl-phenoxy)propyl]-2-methyl-piperidine (229 mg, 701.87 mol, 99.76% yield) as a white solid. MS (ESI) m/z: 328.0 [M+H]⁺

### Step B. Procedure for Preparation of ethyl 2-[(2R,4R)-4-[3-(4-bromo-3-methyl-phenoxy)propyl]-2-methyl-1-piperidyl]acetate

A mixture of (2R,4R)-4-[3-(4-bromo-3-methyl-phenoxy)propyl]-2-methyl-piperidine (229.00 mg, 701.87 μmol, 1 equiv.), ethyl 2-bromoacetate (105.49 mg, 631.68 μmol, 69.91 μL, 0.9 equiv.), K₂CO₃ (291.01 mg, 2.11 mmol, 3 equiv.), and KI (11.65 mg, 70.19 μmol, 0.1 equiv.) in DMF (3 mL) was degassed and purged with N₂ three times. The mixture was stirred at 40° C. for 1 hour under N₂ atmosphere. The reaction mixture was filtered and concentrated under reduced pressure to give a residue. The residue was purified by flash silica gel chromatography (0˜30% ethyl acetate/petroleum ether) to give ethyl 2-[(2R,4R)-4-[3-(4-bromo-3-methyl-phenoxy)propyl]-2-methyl-1-piperidyl]acetate (280 mg, 679.02 μmol, 96.74% yield) as a white solid. MS (ESI) m/z: 412.2 [M+H]⁺

### Step C. Procedure for Preparation of tert-butyl 6-[8-(1,3-benzothiazol-2-ylcarbamoyl)-3,4-dihydro-1H-isoquinolin-2-yl]-3-[4-[3-[(2R,4R)-1-(2-ethoxy-2-oxo-ethyl)-2-methyl-4-piperidyl]propoxy]-2-methyl-phenyl]pyridine-2-carboxylate

A mixture of ethyl 2-[(2R,4R)-4-[3-(4-bromo-3-methyl-phenoxy)propyl]-2-methyl-1-piperidyl]acetate (190.00 mg, 460.76 μmol, 1 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (338.69 mg, 552.92 μmol, 1.2 equiv.), Ad₂nBuP Pd G₃ (67.11 mg, 92.15 mol, 0.2 equiv.), and KF (40.15 mg, 691.14 μmol, 16.19 μL, 1.5 equiv.) in dioxane (2 mL) and H₂O (0.2 mL) was degassed and purged with N₂ three times. The mixture was stirred at 100° C. for 2 hours under N₂ atmosphere. The reaction mixture was filtered and concentrated under reduced pressure to give a residue. The residue was purified by flash silica gel chromatography (0˜50% ethyl acetate/petroleum ether) to give tert-butyl 6-[8-(1,3-benzothiazol-2-ylcarbamoyl)-3,4-dihydro-1H-isoquinolin-2-yl]-3-[4-[3-[(2R,4R)-1-(2-ethoxy-2-oxo-ethyl)-2-methyl-4-piperidyl]propoxy]-2-methyl-phenyl]pyridine-2-carboxylate (310 mg, 378.96 μmol, 82.25% yield) as a white solid. MS (ESI) m/z: 818.4 [M+H]⁺

### Step D. Procedure for Preparation of 2-[(2R,4R)-4-[3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]propyl]-2-methyl-1-piperidyl]acetic acid

A mixture of tert-butyl 6-[8-(1,3-benzothiazol-2-ylcarbamoyl)-3,4-dihydro-1H-isoquinolin-2-yl]-3-[4-[3-[(2R,4R)-1-(2-ethoxy-2-oxo-ethyl)-2-methyl-4-piperidyl]propoxy]-2-methyl-phenyl]pyridine-2-carboxylate (310.00 mg, 378.96 μmol, 1 equiv.) and LiOH·H₂O (79.51 mg, 1.89 mmol, 5 equiv.) in THE (4 mL) and H₂O (2 mL) was degassed and purged with N₂ three times. The mixture was stirred at 25° C. for 12 hours under N₂ atmosphere. The reaction mixture was diluted with H₂O (5 mL) and extracted with DCM (4 mL×3). The combined organic layers were washed with H₂O (4 mL×3), filtered, and concentrated under reduced pressure to give 2-[(2R,4R)-4-[3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]propyl]-2-methyl-1-piperidyl]acetic acid (260 mg, 329.12 μmol, 86.85% yield) as a yellow solid. MS (ESI) m/z: 790.3 [M+H]⁺

### Step E. Procedure for Preparation of tert-butyl 6-[8-(1,3-benzothiazol-2-ylcarbamoyl)-3,4-dihydro-1H-isoquinolin-2-yl]-3-[4-[3-[(2R,4R)-1-[2-[[3-(2,6-dioxo-3-piperidyl)-1-methyl-indazol-6-yl]amino]-2-oxo-ethyl]-2-methyl-4-piperidyl]propoxy]-2-methyl-phenyl]pyridine-2-carboxylate

A mixture of 2-[(2R,4R)-4-[3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]propyl]-2-methyl-1-piperidyl]acetic acid (260.00 mg, 329.12 μmol, 1 equiv.), 3-(6-amino-1-methyl-indazol-3-yl)piperidine-2,6-dione (93.50 mg, 362.03 μmol, 1.1 equiv.), and EDCI (75.71 mg, 394.95 μmol, 1.2 equiv.) in pyridine (3 mL) was degassed and purged with N₂ three times. The mixture was stirred at 40° C. for 1 hour under N₂ atmosphere. The reaction mixture was filtered and concentrated under reduced pressure to give a residue. The residue was purified by flash silica gel chromatography (0˜60% ethyl acetate/petroleum ether) to give tert-butyl 6-[8-(1,3-benzothiazol-2-ylcarbamoyl)-3,4-dihydro-1H-isoquinolin-2-yl]-3-[4-[3-[(2R,4R)-1-[2-[[3-(2,6-dioxo-3-piperidyl)-1-methyl-indazol-6-yl]amino]-2-oxo-ethyl]-2-methyl-4-piperidyl]propoxy]-2-methyl-phenyl]pyridine-2-carboxylate (300 mg, 291.19 μmol, 88.48% yield) as a yellow solid.

MS (ESI) m/z: 1031.2 [M+H]⁺.

### Step F. Procedure for Preparation of 6-[8-(1,3-benzothiazol-2-ylcarbamoyl)-3,4-dihydro-1H-isoquinolin-2-yl]-3-[4-[3-[(2R,4R)-1-[2-[[3-(2,6-dioxo-3-piperidyl)-1-methyl-indazol-6-yl]amino]-2-oxo-ethyl]-2-methyl-4-piperidyl]propoxy]-2-methyl-phenyl]pyridine-2-carboxylic acid

A mixture of tert-butyl 6-[8-(1,3-benzothiazol-2-ylcarbamoyl)-3,4-dihydro-1H-isoquinolin-2-yl]-3-[4-[3-[(2R,4R)-1-[2-[[3-(2,6-dioxo-3-piperidyl)-1-methyl-indazol-6-yl]amino]-2-oxo-ethyl]-2-methyl-4-piperidyl]propoxy]-2-methyl-phenyl]pyridine-2-carboxylate (150 mg, 145.60 μmol, 1 equiv.) and TFA (767.50 mg, 6.73 mmol, 0.5 mL, 46.23 equiv.) in DCM (1.5 mL) was degassed and purged with N₂ three times. The mixture was stirred at 40° C. for 140 min under N₂ atmosphere. The reaction mixture was filtered and concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[4-[3-[(2R,4R)-1-[2-[[3-(2,6-dioxo-3-piperidyl)-1-methyl-indazol-6-yl]amino]-2-oxo-ethyl]-2-methyl-4-piperidyl]propoxy]-2-methyl-phenyl]pyridine-2-carboxylic acid (32 mg, 25.95 μmol, 17.82% yield) as a yellow solid. MS (ESI) m/z: 974.5 [M+H]⁺. ¹H NMR (400 MHz, DMSO-d₆) δ=13.19-12.33 (m, 2H), 10.88 (s, 1H), 9.82 (s, 1H), 8.14 (s, 1H), 8.07-7.98 (m, 2H), 7.79 (d, J=8.0 Hz, 1H), 7.66-7.60 (m, 2H), 7.51-7.41 (m, 3H), 7.40-7.32 (m, 2H), 7.23 (d, J=8.8 Hz, 1H), 6.93 (d, J=8.8 Hz, 2H), 6.79 (d, J=2.4 Hz, 1H), 6.70 (d, J=8.4 Hz, 1H), 4.97 (s, 2H), 4.33 (d, J=9.6 Hz, 1H), 3.94-3.90 (m, 5H), 3.46-3.41 (m, 2H), 3.17 (s, 1H), 3.04-3.01 (m, 2H), 2.94 (s, 2H), 2.78 (s, 2H), 2.45-2.38 (m, 2H), 2.22-2.13 (m, 1H), 2.03 (s, 3H), 1.96 (s, 2H), 1.78-1.70 (m, 2H), 1.68-1.65 (m, 2H), 1.36-1.34 (m, 2H), 1.09-1.00 (m, 5H).

### Example 292. Preparation of Compound 344c

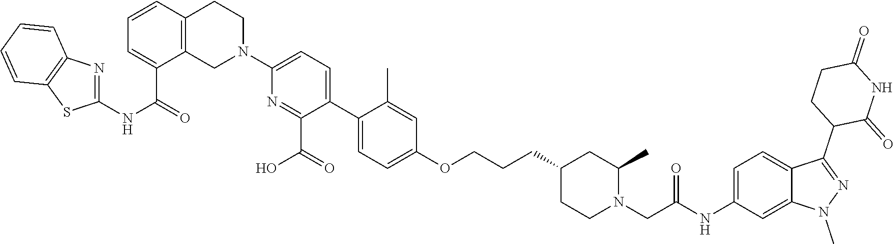 [SMILES: Cc1cc(OCCC[C@H]2CCN(CC(=O)Nc3ccc4c(C5CCC(=O)NC5=O)nn(C)c4c3)[C@H](C)C2)ccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[4-[3-[(2R,4S)-1-[2-[[3-(2,6-dioxo-3-piperidyl)-1-methyl-indazol-6-yl]amino]-2-oxo-ethyl]-2-methyl-4-piperidyl]propoxy]-2-methyl-phenyl]pyridine-2-carboxylic acid

### Step A. Procedure for Preparation of tert-butyl (2R)-4-[3-(4-bromo-3-methyl-phenoxy)propyl]-2-methyl-piperidine-1-carboxylate

To a solution of tert-butyl (2R)-4-(3-hydroxypropyl)-2-methyl-piperidine-1-carboxylate (1.1 g, 4.27 mmol, 1.0 equiv.) in toluene (20 mL) was added 4-bromo-3-methyl-phenol (879.33 mg, 4.70 mmol, 1.1 equiv.) and 2-(tributyl-phosphanylidene) acetonitrile (1.55 g, 6.41 mmol, 1.5 equiv.) at 25° C. The reaction mixture was degassed and purged with N₂ three times, and then stirred at 120° C. for 16 hours under N₂ atmosphere. The reaction mixture was filtered and concentrated under reduced pressure to give a residue. The crude product was purified by reversed-phase HPLC (with 0.1% formic acid) to give tert-butyl (2R)-4-[3-(4-bromo-3-methyl-phenoxy)propyl]-2-methyl-piperidine-1-carboxylate (1.9 g, 4.18 mmol, 48.95% yield) as a colorless oil. MS (ESI) m/z: 448.2 [M+Na]⁺.

### Step B. Procedure for Preparation of tert-butyl (2R,4S)-4-[3-(4-bromo-3-methyl-phenoxy)propyl]-2-methyl-piperidine-1-carboxylate and tert-butyl (2R,4R)-4-[3-(4-bromo-3-methyl-phenoxy)propyl]-2-methyl-piperidine-1-carboxylate

The stereoisomers of tert-butyl (2R)-4-[3-(4-bromo-3-methyl-phenoxy)propyl]-2-methyl-piperidine-1-carboxylate were separated by SFC to give tert-butyl (2R,4R)-4-[3-(4-bromo-3-methyl-phenoxy)propyl]-2-methyl-piperidine-1-carboxylate (310 mg, 727.04 μmol, 16.32% yield) and tert-butyl (2R,4S)-4-[3-(4-bromo-3-methyl-phenoxy)propyl]-2-methyl-piperidine-1-carboxylate (1.15 g, 2.70 mmol, 60.53% yield) as colorless oils. ¹H NMR (400 MHz, DMSO-d₆) δ=7.42 (d, J=8.4 Hz, 1H), 6.94 (d, J=2.8 Hz, 1H), 6.70 (dd, J=3.2, 8.8 Hz, 1H), 3.92 (t, J=6.4 Hz, 2H), 3.81-3.70 (m, 1H), 3.58-3.43 (m, 1H), 3.08-2.98 (m, 1H), 2.29 (s, 3H), 1.83-1.63 (m, 4H), 1.54-1.45 (m, 1H), 1.44-1.33 (m, 11H), 1.19-1.04 (m, 5H). ¹H NMR (400 MHz, DMSO-d₆) δ=7.42 (d, J=8.8 Hz, 1H), 6.94 (d, J=2.8 Hz, 1H), 6.71 (dd, J=3.2, 8.8 Hz, 1H), 4.26 (s, 1H), 3.92 (t, J=6.4 Hz, 2H), 3.80 (d, J=11.2 Hz, 1H), 2.93-2.66 (m, 1H), 2.29 (s, 3H), 1.77-1.58 (m, 4H), 1.57-1.50 (m, 1H), 1.38 (s, 9H), 1.31-1.21 (m, 2H), 1.20-1.10 (m, 1H), 1.06 (d, J=6.8 Hz, 3H), 0.95-0.80 (m, 1H).

### Step C. Procedure for Preparation of (2R,4S)-4-[3-(4-bromo-3-methyl-phenoxy)propyl]-2-methyl-piperidine

To a solution of tert-butyl (2R,4S)-4-[3-(4-bromo-3-methyl-phenoxy)propyl]-2-methyl-piperidine-1-carboxylate (500.00 mg, 1.17 mmol, 1.0 equiv.) in DCM (1.5 mL) was added HCl/dioxane (4 M, 2.50 mL, 8.53 equiv.) at 25° C. The reaction was stirred at 25° C. for 1 hour. The reaction mixture was concentrated under reduced pressure to give (2R,4S)-4-[3-(4-bromo-3-methyl-phenoxy)propyl]-2-methyl-piperidine (460 mg, crude) as a white solid. MS (ESI) m/z: 326.1 [M+H]⁺.

### Step D. Procedure for Preparation of ethyl 2-[(2R, 4S)-4-[3-(4-bromo-3-methyl-phenoxy)propyl]-2-methyl-1-piperidyl]acetate

To a solution of (2R,4S)-4-[3-(4-bromo-3-methyl-phenoxy)propyl]-2-methyl-piperidine (450.00 mg, 1.24 mmol, 1 equiv., HCl) in acetone (4 mL) was added ethyl 2-bromoacetate (207.18 mg, 1.24 mmol, 137.30 μL, 1.0 equiv.) and TEA (251.07 mg, 2.48 mmol, 345.35 μL, 2.0 equiv.) at 25° C. The reaction was stirred at 25° C. for 16 hours. The reaction mixture was filtered and concentrated under reduced pressure to give a residue. The residue was purified by flash silica gel chromatography (0˜50% ethyl acetate/petroleum ether) to give ethyl 2-[(2R, 4S)-4-[3-(4-bromo-3-methyl-phenoxy)propyl]-2-methyl-1-piperidyl]acetate (300 mg, 727.52 μmol, 58.64% yield) as a colorless oil. MS (ESI) m/z: 412.1 [M+H]⁺.

### Step E. Procedure for Preparation of tert-butyl 6-[8-(1,3-benzothiazol-2-ylcarbamoyl)-3,4-dihydro-1H-isoquinolin-2-yl]-3-[4-[3-[(2R,4S)-1-(2-ethoxy-2-oxo-ethyl)-2-methyl-4-piperidyl]propoxy]-2-methyl-phenyl]pyridine-2-carboxylate

A mixture of ethyl 2-[(2R, 4S)-4-[3-(4-bromo-3-methyl-phenoxy)propyl]-2-methyl-1-piperidyl]acetate (290.00 mg, 703.27 μmol, 1.0 equiv.), tert-butyl 6-[8-(1, 3-benzothiazol-2-ylcarbamoyl)-3, 4-dihydro-1H-isoquinolin-2-yl]-3-(4, 4, 5, 5-tetramethyl-1, 3, 2-dioxaborolan-2-yl) pyridine-2-carboxylate (473.86 mg, 773.60 μmol, 1.1 equiv.), KF (122.58 mg, 2.11 mmol, 49.43 μL, 3.0 equiv.), and [2-(2-aminophenyl)phenyl]palladium(1⁺)bis(1-adamantyl)-butyl-phosphanemethanesulfonate (102.43 mg, 140.65 μmol, 0.2 equiv.) in dioxane (3 mL) and H₂O (0.3 mL) was degassed and purged with N₂ three times. The mixture was stirred at 100° C. for 16 hours under N₂ atmosphere. The reaction mixture was filtered and concentrated under reduced pressure to give a residue. The residue was purified by flash silica gel chromatography (0˜6% MeOH/DCM) to give tert-butyl 6-[8-(1,3-benzothiazol-2-ylcarbamoyl)-3,4-dihydro-1H-isoquinolin-2-yl]-3-[4-[3-[(2R,4S)-1-(2-ethoxy-2-oxo-ethyl)-2-methyl-4-piperidyl]propoxy]-2-methyl-phenyl]pyridine-2-carboxylate (400 mg, 327.62 μmol, 46.58% yield) as a yellow solid.

MS (ESI) m/z: 818.5 [M+H]⁺.

### Step F. Procedure for Preparation of 2-[(2R,4S)-4-[3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]propyl]-2-methyl-1-piperidyl]acetic acid

To a solution of tert-butyl 6-[8-(1,3-benzothiazol-2-ylcarbamoyl)-3,4-dihydro-1H-isoquinolin-2-yl]-3-[4-[3-[(2R,4S)-1-(2-ethoxy-2-oxo-ethyl)-2-methyl-4-piperidyl]propoxy]-2-methyl-phenyl]pyridine-2-carboxylate (390 mg, 319.43 μmol, 1.0 equiv.) in THE (4 mL) and H₂O (0.4 mL) was added LiOH·H₂O (40.21 mg, 958.28 μmol, 3.0 equiv.) at 25° C. The reaction was stirred at 25° C. for 16 hours. The reaction mixture was concentrated under reduced pressure to give a residue. The residue was treated with water (30 mL), and its pH was adjusted to ˜5 by progressively adding diluted HCl. The mixture was extracted with DCM/MeOH (10/1) (20 mL×5). The combined organic layers were washed with brine (20 mL×1), dried over Na₂SO₄, filtered and concentrated under reduced pressure to give 2-[(2R,4S)-4-[3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]propyl]-2-methyl-1-piperidyl]acetic acid (250 mg, 233.23 μmol, 73.02% yield) as a yellow solid. MS (ESI) m/z: 790.5 [M+H]⁺.

### Step G. Procedure for Preparation of tert-butyl 6-[8-(1,3-benzothiazol-2-ylcarbamoyl)-3,4-dihydro-1H-isoquinolin-2-yl]-3-[4-[3-[(2R,4S)-1-[2-[[3-(2,6-dioxo-3-piperidyl)-1-methyl-indazol-6-yl]amino]-2-oxo-ethyl]-2-methyl-4-piperidyl]propoxy]-2-methyl-phenyl]pyridine-2-carboxylate

To a solution of 2-[(2R,4S)-4-[3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]propyl]-2-methyl-1-piperidyl]acetic acid (250.00 mg, 316.46 μmol, 1.0 equiv.) in pyridine (3 mL) was added 3-(6-amino-1-methyl-indazol-3-yl) piperidine-2,6-dione (98.08 mg, 379.76 μmol, 1.2 equiv.) and EDCI (78.87 mg, 411.40 μmol, 1.3 equiv.) at 25° C. The reaction was stirred at 25° C. for 16 hours. The mixture was diluted with water (50 mL) and filtered. The organic layer was concentrated to afford a brown residue. The residue was suspended in MeOH (30 mL) and stirred at 15° C. for 30 min. The resulting solid was collected by filtration and dried in vacuo to give tert-butyl 6-[8-(1,3-benzothiazol-2-ylcarbamoyl)-3,4-dihydro-1H-isoquinolin-2-yl]-3-[4-[3-[(2R,4S)-1-[2-[[3-(2,6-dioxo-3-piperidyl)-1-methyl-indazol-6-yl]amino]-2-oxo-ethyl]-2-methyl-4-piperidyl]propoxy]-2-methyl-phenyl]pyridine-2-carboxylate (235 mg, 164.23 μmol, 51.90% yield) as a yellow solid.

MS (ESI) m/z: 1030.6 [M+H]⁺.

### Step H. Procedure for Preparation of 6-[8-(1,3-benzothiazol-2-ylcarbamoyl)-3,4-dihydro-1H-isoquinolin-2-yl]-3-[4-[3-[(2R,4S)-1-[2-[[3-(2,6-dioxo-3-piperidyl)-1-methyl-indazol-6-yl]amino]-2-oxo-ethyl]-2-methyl-4-piperidyl]propoxy]-2-methyl-phenyl]pyridine-2-carboxylic acid

A solution of tert-butyl 6-[8-(1,3-benzothiazol-2-ylcarbamoyl)-3,4-dihydro-1H-isoquinolin-2-yl]-3-[4-[3-[(2R,4S)-1-[2-[[3-(2,6-dioxo-3-piperidyl)-1-methyl-indazol-6-yl]amino]-2-oxo-ethyl]-2-methyl-4-piperidyl]propoxy]-2-methyl-phenyl]pyridine-2-carboxylate (225 mg, 218.40 μmol, 1.0 equiv.) in DCM (1 mL) and TFA (1 mL) was stirred at 40° C. for 2 hours. The reaction mixture was filtered and concentrated under reduced pressure to give a residue. The residue was purified by prep-HPLC to give 6-[8-(1,3-benzothiazol-2-ylcarbamoyl)-3,4-dihydro-1H-isoquinolin-2-yl]-3-[4-[3-[(2R,4S)-1-[2-[[3-(2,6-dioxo-3-piperidyl)-1-methyl-indazol-6-yl]amino]-2-oxo-ethyl]-2-methyl-4-piperidyl]propoxy]-2-methyl-phenyl]pyridine-2-carboxylic acid (49.42 mg, 48.00 μmol, 21.98% yield) as a yellow solid. MS (ESI) m/z: 974.5 [M+H]⁺. ¹H NMR (400 MHz, DMSO-d₆) δ=13.03-12.23 (m, 2H), 10.88 (s, 1H), 10.09-9.67 (m, 1H), 8.08-7.99 (m, 2H), 7.79 (d, J=8.0 Hz, 1H), 7.64 (t, J=8.8 Hz, 2H), 7.51-7.41 (m, 3H), 7.41-7.32 (m, 2H), 7.21 (d, J=8.8 Hz, 1H), 6.95 (d, J=8.8 Hz, 1H), 6.92 (d, J=8.4 Hz, 1H), 6.79 (s, 1H), 6.71 (d, J=8.0 Hz, 1H), 4.97 (s, 2H), 4.33 (dd, J=4.8, 9.6 Hz, 1H), 3.97-3.89 (m, 7H), 3.02 (t, J=5.2 Hz, 2H), 2.75-2.57 (m, 4H), 2.42-2.28 (m, 2H), 2.17 (dd, J=5.2, 12.8 Hz, 1H), 2.03 (s, 3H), 1.79-1.53 (m, 7H), 1.46-1.27 (m, 4H), 1.13-1.00 (m, 3H).

### Example 293. Preparation of Compound 344d

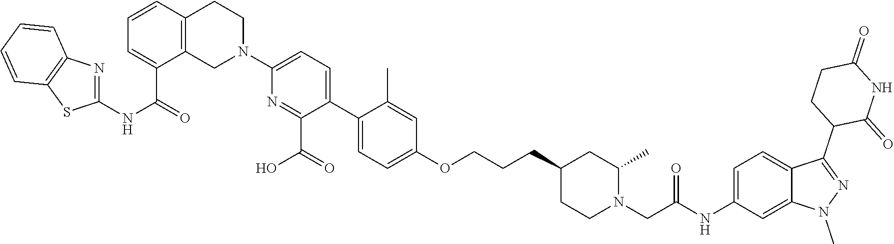 [SMILES: Cc1cc(OCCC[C@@H]2CCN(CC(=O)Nc3ccc4c(C5CCC(=O)NC5=O)nn(C)c4c3)[C@@H](C)C2)ccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(4-(3-((2S,4R)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinic acid

### Step A. Procedure for Preparation of tert-butyl (S,E)-4-(3-(benzyloxy)propylidene)-2-methylpiperidine-1-carboxylate

To a solution of 3-benzyloxypropyl(triphenyl)phosphoniumbromide (17.28 g, 35.17 mmol, 1.5 equiv.) in THE (50 mL) was added LiHMDS (1 M, 46.89 mL, 2.0 equiv.) at 0° C. The reaction mixture was stirred for 1 hour under N₂ atmosphere, then tert-butyl (2S)-2-methyl-4-oxo-piperidine-1-carboxylate (5 g, 23.44 mmol, 1.0 equiv.) in THF (50 mL) was added dropwise at 0° C. The resulting mixture was stirred at 25° C. for 15 hours under N₂ atmosphere. The reaction mixture was quenched by addition of saturated NH₄Cl 50 mL at 0° C., and then extracted with ethyl acetate (50 mL×3). The combined organic layers were washed with H₂O (100 mL), dried over Na₂SO₄, filtered, and concentrated under reduced pressure to give a residue. The residue was purified by flash silica gel chromatography (0˜6% ethyl acetate/petroleum ether) to give tert-butyl (S,E)-4-(3-(benzyloxy)propylidene)-2-methylpiperidine-1-carboxylate (3.4 g, 9.84 mmol, 41.98% yield) as a colorless oil. ¹H NMR (400 MHz, DMSO-d₆) δ=7.43-7.22 (m, 5H), 5.42-5.20 (m, 1H), 4.47-4.40 (m, 2H), 4.37-4.19 (m, 1H), 4.05-3.77 (m, 1H), 3.43-3.38 (m, 2H), 3.35-3.30 (m, 1H), 2.83-2.64 (m, 1H), 2.44-1.74 (m, 6H), 1.45-1.36 (m, 9H), 1.09-1.07 (m, 1H), 0.97-0.92 (m, 2H)

### Step B. Procedure for Preparation of tert-butyl (2S)-4-(3-hydroxypropyl)-2-methylpiperidine-1-carboxylate

A mixture of tert-butyl (S,E)-4-(3-(benzyloxy)propylidene)-2-methylpiperidine-1-carboxylate (3.4 g, 9.84 mmol, 1.0 equiv.), Pd/C (1.5 g, 1.41 mmol, 10% purity, 0.143 equiv.), and Pd(OH)₂ (1.5 g, 2.14 mmol, 20% purity, 0.217 equiv.) in EtOH (34 mL) was degassed and purged with N₂ three times. The mixture was stirred at 60° C. for 16 hours under N₂ atmosphere. The combined organic layers were filtered and concentrated under reduced pressure to give tert-butyl (2S)-4-(3-hydroxypropyl)-2-methylpiperidine-1-carboxylate (2.4 g, crude) as a yellow oil, which was used in the next step without further purification. ¹H NMR (400 MHz, DMSO-d₆) δ=4.42-4.17 (m, 2H), 3.86-3.71 (m, 1H), 2.90-2.64 (m, 1H), 1.70-1.47 (m, 3H), 1.43-1.37 (m, 13H), 1.30-1.11 (m, 4H), 1.10-1.04 (m, 3H).

### Step C. Procedure for Preparation of tert-butyl (2S)-4-(3-(4-bromo-3-methylphenoxy)propyl)-2-methylpiperidine-1-carboxylate

A mixture of tert-butyl (2S)-4-(3-hydroxypropyl)-2-methylpiperidine-1-carboxylate (2.3 g, 8.94 mmol, 1.1 equiv.), 4-bromo-3-methyl-phenol (1.52 g, 8.12 mmol, 1.0 equiv.), 2-(tributyl-phosphanylidene)acetonitrile (2.94 g, 12.19 mmol, 1.5 equiv.) in toluene (23 mL) was degassed and purged with N₂ three times. The mixture was stirred at 120° C. for 16 hours under N₂ atmosphere. The combined organic layers were filtered and concentrated under reduced pressure to give a residue. The residue was purified by flash silica gel chromatography (0˜3% ethyl acetate/petroleum ether) to give tert-butyl (2S)-4-(3-(4-bromo-3-methylphenoxy)propyl)-2-methylpiperidine-1-carboxylate (1.95 g, 4.37 mmol, 53.79% yield) as a white solid.

### Step D. Procedure for Preparation of tert-butyl (2S,4S)-4-(3-(4-bromo-3-methylphenoxy)propyl)-2-methylpiperidine-1-carboxylate and tert-butyl (2S,4R)-4-(3-(4-bromo-3-methylphenoxy)propyl)-2-methylpiperidine-1-carboxylate

The stereoisomers of tert-butyl (2S)-4-(3-(4-bromo-3-methylphenoxy)propyl)-2-methylpiperidine-1-carboxylate were separated by prep-HPLC (column: DAICEL CHIRALPAK IG (250 mm×30 mm, 10 um); mobile phase: [CO₂-I-PrOH (0.1% NH₃H₂O)]; B %: 15%, isocratic elution mode and column: DAICEL CHIRALPAK IG (250 mm×30 mm, 10 um); mobile phase: [CO₂-CAN/I-PrOH(0.1% NH₃H₂O)]; B %: 20%, isocratic elution mode) to give tert-butyl (2S,4S)-4-(3-(4-bromo-3-methylphenoxy)propyl)-2-methylpiperidine-1-carboxylate (293 mg, 687.17 μmol, 15.42% yield) and tert-butyl (2S,4R)-4-(3-(4-bromo-3-methylphenoxy)propyl)-2-methylpiperidine-1-carboxylate (650 mg, 1.52 mmol, 34.21% yield) as a colorless oil. MS (ESI) m/z: 327.8 [M+H]⁺. ¹H NMR (400 MHz, DMSO-d₆) δ=7.42 (d, J=8.8 Hz, 1H), 6.94 (d, J=2.8 Hz, 1H), 6.70 (dd, J=2.8, 8.8 Hz, 1H), 3.92 (t, J=6.4 Hz, 2H), 3.81-3.72 (m, 1H), 3.56-3.51 (m, 1H), 3.07-3.00 (m, 1H), 2.29 (s, 3H), 1.77-1.67 (m, 4H), 1.52-1.45 (m, 1H), 1.38 (s, 11H), 1.16-1.09 (m, 5H). ¹H NMR (400 MHz, DMSO-d₆) δ=7.42 (d, J=8.8 Hz, 1H), 6.94 (d, J=2.8 Hz, 1H), 6.71 (dd, J=2.8, 8.8 Hz, 1H), 4.27 (s, 1H), 3.92 (t, J=6.4 Hz, 2H), 3.80 (d, J=12.0 Hz, 1H), 2.90-2.70 (m, 1H), 2.29 (s, 3H), 1.75-1.62 (m, 4H), 1.58-1.52 (m, 1H), 1.40-1.37 (m, 9H), 1.31-1.24 (m, 2H), 1.19-1.12 (m, 1H), 1.06 (d, J=6.8 Hz, 3H), 0.95-0.84 (m, 1H)

### Step E. Procedure for Preparation of (2S,4R)-4-(3-(4-bromo-3-methylphenoxy)propyl)-2-methylpiperidine

A solution of tert-butyl (2S,4R)-4-(3-(4-bromo-3-methylphenoxy)propyl)-2-methylpiperidine-1-carboxylate (360.00 mg, 844.30 μmol, 1.0 equiv.) in HCl/dioxane (4 mL) was stirred at 25° C. for 2 hours. The reaction mixture was filtered and concentrated under reduced pressure to give (2S,4R)-4-(3-(4-bromo-3-methylphenoxy)propyl)-2-methylpiperidine (300 mg, crude) as a white solid, which was used in the next step without further purification. MS (ESI) m/z: 327.8 [M+H]⁺.

### Step F. Procedure for Preparation of ethyl 2-((2S,4R)-4-(3-(4-bromo-3-methylphenoxy)propyl)-2-methylpiperidin-1-yl)acetate

A mixture of (2S,4R)-4-(3-(4-bromo-3-methylphenoxy)propyl)-2-methylpiperidine (270.00 mg, 744.35 μmol, 1.0 equiv., HCl), ethyl 2-bromoacetate (124.31 mg, 744.35 μmol, 82.38 μL, 1.0 equiv.), and TEA (150.64 mg, 1.49 mmol, 207.21 μL, 2.0 equiv.) in acetone (3 mL) was stirred at 25° C. for 1 hour. The reaction mixture was filtered and concentrated under reduced pressure to give a residue. The residue was purified by flash silica gel chromatography (0˜100% ethyl acetate/petroleum ether) to give ethyl 2-((2S,4R)-4-(3-(4-bromo-3-methylphenoxy)propyl)-2-methylpiperidin-1-yl)acetate (300 mg, 727.52 μmol, 97.74% yield) as a white solid. MS (ESI) m/z: 414.0 [M+H]⁺.

### Step G. Procedure for Preparation of tert-butyl 6-(8-(benzo[d]thiazol-2-ylcarbamoyl)-3,4-dihydroisoquinolin-2(1H)-yl)-3-(4-(3-((2S,4R)-1-(2-ethoxy-2-oxoethyl)-2-methylpiperidin-4-yl)propoxy)-2-methylphenyl)picolinate

A mixture of ethyl 2-((2S,4R)-4-(3-(4-bromo-3-methylphenoxy)propyl)-2-methylpiperidin-1-yl)acetate (300 mg, 727.52 μmol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (534.77 mg, 873.02 μmol, 1.2 equiv.), KF (1.5 M, 1.46 mL, 3.0 equiv.), and [2-(2-aminophenyl)phenyl]palladium(1⁺)bis(1-adamantyl)-butyl-phosphane methanesulfonate (52.98 mg, 72.75 μmol, 0.1 equiv.) in dioxane (4 mL) was degassed and purged with N₂ three times. The mixture was stirred at 100° C. for 1 hour under N₂ atmosphere in a microwave reactor. The combined organic layers were filtered and concentrated under reduced pressure to give a residue. The residue was purified by flash silica gel chromatography (0˜70% ethyl acetate/petroleum ether) to give tert-butyl 6-(8-(benzo[d]thiazol-2-ylcarbamoyl)-3,4-dihydroisoquinolin-2(1H)-yl)-3-(4-(3-((2S,4R)-1-(2-ethoxy-2-oxoethyl)-2-methylpiperidin-4-yl)propoxy)-2-methylphenyl)picolinate (600 mg, crude) as a yellow solid. MS (ESI) m/z: 818.7 [M+H]⁺.

### Step H. Procedure for Preparation of 2-((2S,4R)-4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)propyl)-2-methylpiperidin-1-yl)acetic acid

A mixture of tert-butyl 6-(8-(benzo[d]thiazol-2-ylcarbamoyl)-3,4-dihydroisoquinolin-2(1H)-yl)-3-(4-(3-((2S,4R)-1-(2-ethoxy-2-oxoethyl)-2-methylpiperidin-4-yl)propoxy)-2-methylphenyl)picolinate (600.00 mg, 733.47 μmol, 1.0 equiv.), LiOH·H₂O (92.34 mg, 2.20 mmol, 3.0 equiv.) in H₂O (2 mL) and THE (6 mL) was stirred at 25° C. for 2 hours. The reaction mixture was diluted with H₂O (5 mL) and concentrated to a turbid liquid. Then the pH of the turbid liquid was adjusted to pH=3 with 1 N HCl (5 mL). The solution was extracted with DCM/MeOH (20:1) and the combined organic layers were filtered and concentrated to give 2-((2S,4R)-4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)propyl)-2-methylpiperidin-1-yl)acetic acid (550 mg, 696.22 μmol, 94.92% yield) as a yellow solid, which was used in the next step without further purification. MS (ESI) m/z: 790.7 [M+H]⁺.

### Step I. Procedure for preparation of tert-butyl 6-(8-(benzo[d]thiazol-2-ylcarbamoyl)-3,4-dihydroisoquinolin-2(1H)-yl)-3-(4-(3-((2S,4R)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinate

A mixture of 2-((2S,4R)-4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)propyl)-2-methylpiperidin-1-yl)acetic acid (200.00 mg, 253.17 μmol, 1.0 equiv.), 3-(6-amino-1-methyl-indazol-3-yl)piperidine-2,6-dione (78.47 mg, 303.81 μmol, 1.2 equiv.) and EDCI (72.80 mg, 379.76 μmol, 1.5 equiv.) in pyridine (2 mL) was stirred at 25° C. for 2 hours. The mixture was diluted with H₂O (5 mL×3) and extracted with ethyl acetate (10 mL×3). The combined organic layers were filtered and concentrated under reduced pressure to give tert-butyl 6-(8-(benzo[d]thiazol-2-ylcarbamoyl)-3,4-dihydroisoquinolin-2(1H)-yl)-3-(4-(3-((2S,4R)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinate (200 mg, crude) as a yellow solid, which was used in the next step without further purification.

MS (ESI) m/z: 1030.5 [M+H]⁺.

### Step J. Procedure for Preparation of 6-(8-(benzo[d]thiazol-2-ylcarbamoyl)-3,4-dihydroisoquinolin-2(1H)-yl)-3-(4-(3-((2S,4R)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinic acid

A solution of tert-butyl 6-(8-(benzo[d]thiazol-2-ylcarbamoyl)-3,4-dihydroisoquinolin-2(1H)-yl)-3-(4-(3-((2S,4R)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinate (200.00 mg, 194.13 μmol, 1.0 equiv.) in TFA (1 mL) and DCM (1 mL) was stirred at 25° C. for 16 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(4-(3-((2S,4R)-1-(2-((3-(2,6-dioxopiperidin-3-yl)-1-methyl-1H-indazol-6-yl)amino)-2-oxoethyl)-2-methylpiperidin-4-yl)propoxy)-2-methylphenyl)picolinic acid (98.90 mg, 98.35 μmol, 50.66% yield) as a yellow solid. MS (ESI) m/z: 974.5[M+H]⁺. ¹H NMR (400 MHz, DMSO-d₆) δ=13.02-12.76 (m, 1H), 12.73-12.39 (m, 1H), 10.89 (s, 1H), 9.99-9.24 (m, 1H), 8.07-7.99 (m, 2H), 7.79 (d, J=8.0 Hz, 1H), 7.72-7.60 (m, 2H), 7.51-7.42 (m, 3H), 7.41-7.31 (m, 2H), 7.21-7.10 (m, 1H), 6.94 (dd, J=8.8, 13.6 Hz, 2H), 6.81-6.77 (m, 1H), 6.74-6.69 (m, 1H), 4.97 (s, 2H), 4.34 (dd, J=5.2, 9.6 Hz, 1H), 4.22-4.04 (m, 1H), 3.98-3.89 (m, 7H), 3.03 (t, J=5.6 Hz, 2H), 2.68-2.61 (m, 2H), 2.56-2.51 (m, 4H), 2.37-2.31 (m, 1H), 2.24-2.14 (m, 1H), 2.03 (s, 3H), 1.90-1.60 (m, 6H), 1.53-1.20 (m, 6H).

### Example 294. Preparation of Compound 346a

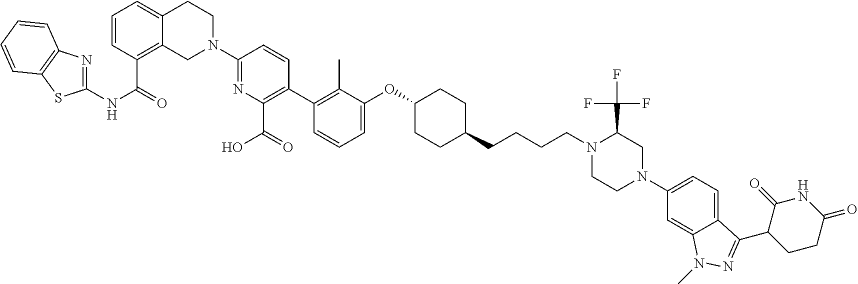 [SMILES: Cc1c(O[C@H]2CC[C@H](CCCCN3CCN(c4ccc5c(C6CCC(=O)NC6=O)nn(C)c5c4)C[C@@H]3C(F)(F)F)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(4-((2R)-4-(3-(2,6-dioxopiperidin-3-yl)-1-methyl-1H-indazol-6-yl)-2-(trifluoromethyl)piperazin-1-yl)butyl)cyclohexyl)oxy)-2-methylphenyl)picolinic acid

### Step A. Procedure for Preparation of tert-butyl (2R)-4-[3-(2,6-dibenzyloxy-3-pyridyl)-1-methyl-indazol-6-yl]-2-(trifluoromethyl)piperazine-1-carboxylate

A mixture of 6-bromo-3-(2,6-dibenzyloxy-3-pyridyl)-1-methyl-indazole (650 mg, 1.30 mmol, 1.0 equiv.), tert-butyl (2R)-2-(trifluoromethyl)piperazine-1-carboxylate (495.40 mg, 1.95 mmol, 1.5 equiv.), Pd₂(dba)₃ (118.95 mg, 129.90 μmol, 0.1 equiv.), RuPhos (121.23 mg, 259.80 μmol, 0.2 equiv.), and Cs₂CO₃ (1.27 g, 3.90 mmol, 3.0 equiv.) in toluene (10 mL) was degassed and purged with N₂ three times. The mixture was stirred at 100° C. for 16 hours under N₂ atmosphere. The reaction mixture was filtered and concentrated under reduced pressure to give a residue. The residue was purified by flash silica gel chromatography (0˜23% ethyl acetate/petroleum ether) to afford tert-butyl (2R)-4-[3-(2,6-dibenzyloxy-3-pyridyl)-1-methyl-indazol-6-yl]-2-(trifluoromethyl)piperazine-1-carboxylate (700 mg, 997.44 μmol, 76.79% yield) as a yellow oil.

MS (ESI) m/z: 674.3 [M+H]⁺.

### Step B. Procedure for Preparation of tert-butyl (2R)-4-[3-(2,6-dioxo-3-piperidyl)-1-methyl-indazol-6-yl]-2-(trifluoromethyl)piperazine-1-carboxylate

To a solution of tert-butyl (2R)-4-[3-(2,6-dibenzyloxy-3-pyridyl)-1-methyl-indazol-6-yl]-2-(trifluoromethyl)piperazine-1-carboxylate (700 mg, 1.04 mmol, 1.0 equiv.) in THE (10 mL) and EtOH (10 mL) was added Pd/C (350 mg, 328.89 μmol, 10% purity, 0.317 equiv.) and Pd(OH)₂ (350 mg, 2.49 mmol, 2.40 equiv.) under N₂ atmosphere. The suspension was degassed and purged with H₂ three times. The mixture was stirred under H₂ (15 Psi) at 40° C. for 16 hours. The reaction mixture was filtered and washed with THE (100 mL). The filtrate was concentrated under reduced pressure to afford tert-butyl (2R)-4-[3-(2,6-dioxo-3-piperidyl)-1-methyl-indazol-6-yl]-2-(trifluoromethyl)piperazine-1-carboxylate (600 mg, crude) as a yellow oil, which was used in the next step without further purification. MS (ESI) m/z: 496.4 [M+H]⁺.

### Step C. Procedure for Preparation of 3-[1-methyl-6-[(3R)-3-(trifluoromethyl)piperazin-1-yl]indazol-3-yl]piperidine-2,6-dione

A solution of tert-butyl (2R)-4-[3-(2,6-dioxo-3-piperidyl)-1-methyl-indazol-6-yl]-2-(trifluoromethyl)piperazine-1-carboxylate (600 mg, 1.21 mmol, 1.0 equiv.) in HCl/dioxane (10 mL) was stirred at 25° C. for 16 hours. The reaction mixture was filtered to afford 3-[1-methyl-6-[(3R)-3-(trifluoromethyl)piperazin-1-yl]indazol-3-yl]piperidine-2,6-dione (400 mg, crude) as a white solid, which was used in the next step without further purification. MS (ESI) m/z: 396.1 [M+H]⁺.

¹H NMR (400 MHz, DMSO-d₆) δ=10.86 (s, 1H), 7.59 (d, J=8.8 Hz, 1H), 7.10 (s, 1H), 7.00 (dd, J=1.2, 9.2 Hz, 1H), 4.70-4.61 (m, 1H), 4.07-3.99 (m, 2H), 3.94 (s, 3H), 3.37-3.14 (m, 5H), 2.65-2.58 (m, 2H), 2.32 (J=4.8, 8.4 Hz, 1H), 2.15 (dd, J=5.6, 13.6 Hz, 1H)

### Step D. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(4-((2R)-4-(3-(2,6-dioxopiperidin-3-yl)-1-methyl-1H-indazol-6-yl)-2-(trifluoromethyl)piperazin-1-yl)butyl)cyclohexyl)oxy)-2-methylphenyl)picolinate

To a solution of tert-butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate (100 mg, 134.24 mol, 1.0 equiv.) and 3-[1-methyl-6-[(3R)-3-(trifluoromethyl)piperazin-1-yl]indazol-3-yl]piperidine-2,6-dione (63.69 mg, 161.09 μmol, 1.2 equiv.) in DCM (5 mL) was added NaBH(OAc)₃ (85.35 mg, 402.72 μmol, 3.0 equiv.). The mixture was stirred at 25° C. for 5 hours. The reaction mixture was diluted with H₂O (30 mL) and extracted with ethyl acetate (20 mL×3). The combined organic layers were washed with brine (50 mL), dried over Na₂SO₄, filtered, and concentrated under reduced pressure to give a residue. The residue was purified by prep-TLC (dichloromethane:methanol=15:1) to afford tert-butyl 6-(8-(benzo[d]thiazol-2-ylcarbamoyl)-3,4-dihydroisoquinolin-2(1H)-yl)-3-(3-(((1s,4r)-4-(4-((2R)-4-(3-(2,6-dioxopiperidin-3-yl)-1-methyl-1H-indazol-6-yl)-2-(trifluoromethyl)piperazin-1-yl)butyl)cyclohexyl)oxy)-2-methylphenyl)picolinate (70 mg, 44.20 μmol, 32.93% yield) as a light yellow solid. MS (ESI)m/z: 1124.5 [M+H]⁺.

### Step E. Procedure for Preparation of 6-(8-(benzo[d]thiazol-2-ylcarbamoyl)-3,4-dihydroisoquinolin-2(1H)-yl)-3-(3-(((1s,4r)-4-(4-((2R)-4-(3-(2,6-dioxopiperidin-3-yl)-1-methyl-1H-indazol-6-yl)-2-(trifluoromethyl)piperazin-1-yl)butyl)cyclohexyl)oxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(((1s,4r)-4-(4-((2R)-4-(3-(2,6-dioxopiperidin-3-yl)-1-methyl-1H-indazol-6-yl)-2-(trifluoromethyl)piperazin-1-yl)butyl)cyclohexyl)oxy)-2-methylphenyl)picolinate (70 mg, 62.26 mol, 1.0 equiv.) in DCM (1 mL) was added TFA (3.07 g, 26.92 mmol, 2 mL, 432.45 equiv.). The mixture was stirred at 25° C. for 16 hours. The reaction mixture was then concentrated under reduced pressure to give a residue. The residue was purified by prep-HPLC to afford 6-(8-(benzo[d]thiazol-2-ylcarbamoyl)-3,4-dihydroisoquinolin-2(1H)-yl)-3-(3-(((1s,4r)-4-(4-((2R)-4-(3-(2,6-dioxopiperidin-3-yl)-1-methyl-1H-indazol-6-yl)-2-(trifluoromethyl)piperazin-1-yl)butyl)cyclohexyl)oxy)-2-methylphenyl)picolinic acid (24.17 mg, 22.36 μmol, 35.91% yield) as a white solid. MS (ESI) m/z: 1068.5 [M+H]⁺. ¹H NMR (400 MHz, DMSO-d₆) δ=12.91-12.83 (m, 1H), 10.86 (s, 1H), 8.04 (d, J=8.0 Hz, 1H), 7.80 (d, J=7.6 Hz, 1H), 7.63 (d, J=6.4 Hz, 1H), 7.54-7.49 (m, 1H), 7.49-7.43 (m, 3H), 7.40-7.33 (m, 2H), 7.12-7.03 (m, 1H), 6.95 (dd, J=8.4, 18.4 Hz, 2H), 6.87-6.82 (m, 2H), 6.61 (d, J=7.2 Hz, 1H), 4.98 (s, 2H), 4.27 (dd, J=5.2, 9.2 Hz, 1H), 4.23-4.15 (m, 1H), 3.90 (s, 4H), 3.64 (d, J=9.6 Hz, 2H), 3.03 (t, J=5.6 Hz, 2H), 2.88-2.82 (m, 1H), 2.75 (d, J=6.4 Hz, 2H), 2.67 (d, J=1.6 Hz, 1H), 2.61 (t, J=6.0 Hz, 2H), 2.35-2.32 (m, 2H), 2.19-2.15 (m, 1H), 2.11-2.05 (m, 2H), 1.87 (s, 3H), 1.79 (d, J=12.8 Hz, 2H), 1.49 (dd, J=4.0, 6.0 Hz, 2H), 1.40-1.20 (m, 9H), 1.11-1.02 (m, 2H)

### Example 295. Preparation of Compound 346b

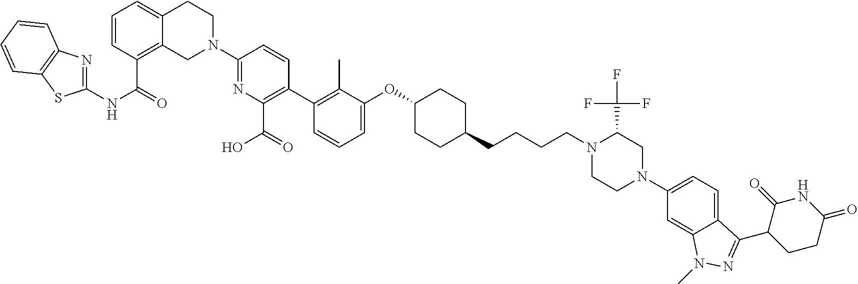 [SMILES: Cc1c(O[C@H]2CC[C@H](CCCCN3CCN(c4ccc5c(C6CCC(=O)NC6=O)nn(C)c5c4)C[C@H]3C(F)(F)F)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-(4-((2S)-4-(3-(2,6-dioxopiperidin-3-yl)-1-methyl-1H-indazol-6-yl)-2-(trifluoromethyl)piperazin-1-yl)butyl)cyclohexyl)oxy)-2-methylphenyl)picolinic acid

### Step A. Procedure for Preparation of tert-butyl 4-[3-(2,6-dibenzyloxy-3-pyridyl)-1-methyl-indazol-6-yl]-2-(trifluoromethyl)piperazine-1-carboxylate

A mixture of 6-bromo-3-(2,6-dibenzyloxy-3-pyridyl)-1-methyl-indazole (1.3 g, 2.60 mmol, 1.0 equiv.), tert-butyl 2-(trifluoromethyl)piperazine-1-carboxylate (990.81 mg, 3.90 mmol, 1.5 equiv.), Pd₂(dba)₃ (237.90 mg, 259.80 μmol, 0.1 equiv.), RuPhos (242.47 mg, 519.60 μmol, 0.2 equiv.) and Cs₂CO₃ (2.54 g, 7.79 mmol, 3 equiv.) in toluene (20 mL) was degassed and purged with N₂ three times. The mixture was stirred at 100° C. for 16 hours under a N₂ atmosphere. The reaction mixture was filtered and concentrated under pressure to give a residue. The residue was purified by flash silica gel chromatography (0˜24% ethyl acetate/petroleum ether) to give tert-butyl 4-[3-(2,6-dibenzyloxy-3-pyridyl)-1-methyl-indazol-6-yl]-2-(trifluoromethyl)piperazine-1-carboxylate (1.1 g, 1.55 mmol, 59.70% yield) as a yellow oil. MS (ESI) m/z: 674.3 [M+H]⁺.

### Step B. Procedure for Preparation of tert-butyl (2R)-4-[3-(2,6-dibenzyloxy-3-pyridyl)-1-methyl-indazol-6-yl]-2-(trifluoromethyl)piperazine-1-carboxylate and tert-butyl (2S)-4-[3-(2,6-dibenzyloxy-3-pyridyl)-1-methyl-indazol-6-yl]-2-(trifluoromethyl)piperazine-1-carboxylate

The stereoisomers of tert-butyl 4-[3-(2,6-dibenzyloxy-3-pyridyl)-1-methyl-indazol-6-yl]-2-(trifluoromethyl)piperazine-1-carboxylate mixture were separated by SFC to afford tert-butyl (2R)-4-[3-(2,6-dibenzyloxy-3-pyridyl)-1-methyl-indazol-6-yl]-2-(trifluoromethyl)piperazine-1-carboxylate (390 mg, 578.87 μmol, 35.45% yield) as a white solid and tert-butyl (2S)-4-[3-(2,6-dibenzyloxy-3-pyridyl)-1-methyl-indazol-6-yl]-2-(trifluoromethyl)piperazine-1-carboxylate (290 mg, 430.44 μmol, 26.36% yield) as a light yellow solid.

### Step C. Procedure for Preparation of tert-butyl (2S)-4-[3-(2,6-dioxo-3-piperidyl)-1-methyl-indazol-6-yl]-2-(trifluoromethyl)piperazine-1-carboxylate

To a solution of tert-butyl (2S)-4-[3-(2,6-dibenzyloxy-3-pyridyl)-1-methyl-indazol-6-yl]-2-(trifluoromethyl)piperazine-1-carboxylate (290 mg, 430.44 μmol, 1 equiv.) in THE (5 mL) and EtOH (5 mL) was added Pd/C (150 mg, 140.95 μmol, 10% purity, 0.327 equiv.) and Pd(OH)₂ (150 mg, 1.07 mmol, 2.48 equiv.) under N₂ atmosphere. The suspension was degassed and purged with H₂ three times. The mixture was stirred under H₂ (15 Psi) at 40° C. for 16 hours. The reaction mixture was filtered and washed with THE (50 mL). The filtrate was concentrated to give tert-butyl (2S)-4-[3-(2,6-dioxo-3-piperidyl)-1-methyl-indazol-6-yl]-2-(trifluoromethyl)piperazine-1-carboxylate (260 mg, crude) as a yellow oil, which was used in the next step without further purification. MS (ESI) m/z: 496.3 [M+H]⁺.

### Step D. Procedure for Preparation of 3-[1-methyl-6-[(3S)-3-(trifluoromethyl)piperazin-1-yl]indazol-3-yl]piperidine-2,6-dione

A solution of tert-butyl (2S)-4-[3-(2,6-dioxo-3-piperidyl)-1-methyl-indazol-6-yl]-2-(trifluoromethyl)piperazine-1-carboxylate (260.00 mg, 524.73 μmol, 1.0 equiv.) in HCl/dioxane (10 mL) was stirred at 25° C. for 16 hours. The reaction mixture was filtered to give 3-[1-methyl-6-[(3S)-3-(trifluoromethyl)piperazin-1-yl]indazol-3-yl]piperidine-2,6-dione (170 mg, crude) as a light-yellow solid, which was used in the next step without further purification. MS (ESI) m/z: 396.2 [M+H]⁺. 1H NMR (400 MHz, DMSO-d₆) δ=10.87 (s, 1H), 7.59 (d, J=8.8 Hz, 1H), 7.11 (s, 1H), 7.01 (dd, J=1.6, 8.8 Hz, 1H), 4.72-4.60 (m, 1H), 4.30 (dd, J=5.2, 9.6 Hz, 1H), 4.03 (d, J=11.2 Hz, 1H), 3.94 (s, 3H), 3.90 (d, J=12.4 Hz, 1H), 3.48-3.42 (m, 1H), 3.36-3.18 (m, 3H), 2.72-2.55 (m, 2H), 2.39-2.27 (m, 1H), 2.22-2.10 (m, 1H)

### Step E. Procedure for Preparation of tert-butyl 6-(8-(benzo[d]thiazol-2-ylcarbamoyl)-3,4-dihydroisoquinolin-2(1H)-yl)-3-(3-(((1r,4r)-4-(4-((2S)-4-(3-(2,6-dioxopiperidin-3-yl)-1-methyl-1H-indazol-6-yl)-2-(trifluoromethyl)piperazin-1-yl)butyl)cyclohexyl)oxy)-2-methylphenyl)picolinate

To a solution of tert-butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate (100 mg, 134.24 mol, 1.0 equiv.) and 3-[1-methyl-6-[(3S)-3-(trifluoromethyl)piperazin-1-yl]indazol-3-yl]piperidine-2,6-dione (58.38 mg, 147.66 μmol, 1.1 equiv.) in DCM (5 mL) was added NaBH(OAc)₃ (85.35 mg, 402.72 μmol, 3.0 equiv.). The mixture was stirred at 25° C. for 16 hours. The reaction mixture was then diluted with H₂O (30 mL) and extracted with ethyl acetate (20 mL×3). The combined organic layers were washed with brine (50 mL), dried over Na₂SO₄, filtered and concentrated under reduced pressure to give tert-butyl 6-(8-(benzo[d]thiazol-2-ylcarbamoyl)-3,4-dihydroisoquinolin-2(1H)-yl)-3-(3-(((1r,4r)-4-(4-((2S)-4-(3-(2,6-dioxopiperidin-3-yl)-1-methyl-1H-indazol-6-yl)-2-(trifluoromethyl)piperazin-1-yl)butyl)cyclohexyl)oxy)-2-methylphenyl)picolinate (150 mg, crude) as a light yellow solid, which was used in the next step without further purification. MS (ESI) m/z: 1125.8 [M+H]⁺.

### Step F. Procedure for Preparation of 6-(8-(benzo[d]thiazol-2-ylcarbamoyl)-3,4-dihydroisoquinolin-2(1H)-yl)-3-(3-(((1r,4r)-4-(4-((2S)-4-(3-(2,6-dioxopiperidin-3-yl)-1-methyl-1H-indazol-6-yl)-2-(trifluoromethyl)piperazin-1-yl)butyl)cyclohexyl)oxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(((1r,4r)-4-(4-((2S)-4-(3-(2,6-dioxopiperidin-3-yl)-1-methyl-1H-indazol-6-yl)-2-(trifluoromethyl)piperazin-1-yl)butyl)cyclohexyl)oxy)-2-methylphenyl)picolinate (150 mg, 133.41 μmol, 1.0 equiv.) in DCM (3 mL) was added TFA (9.21 g, 80.77 mmol, 6 mL, 605.43 equiv.). The mixture was stirred at 30° C. for 2 hours. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1r,4r)-4-(4-((2S)-4-(3-(2,6-dioxopiperidin-3-yl)-1-methyl-1H-indazol-6-yl)-2-(trifluoromethyl)piperazin-1-yl)butyl)cyclohexyl)oxy)-2-methylphenyl)picolinic acid (56.36 mg, 51.33 μmol, 38.47% yield) as a white solid. MS (ESI) m/z: 1068.3 [M+H]⁺. ¹H NMR (400 MHz, DMSO-d₆) δ=12.91-12.81 (m, 1H), 10.85 (s, 1H), 8.03 (d, J=8.0 Hz, 1H), 7.79 (d, J=8.0 Hz, 1H), 7.62 (d, J=7.6 Hz, 1H), 7.53-7.48 (m, 1H), 7.48-7.42 (m, 3H), 7.39-7.32 (m, 2H), 7.10-7.03 (m, 1H), 6.94 (dd, J=8.4, 18.4 Hz, 2H), 6.87-6.81 (m, 2H), 6.61 (d, J=7.2 Hz, 1H), 4.98 (s, 2H), 4.26 (dd, J=5.2, 9.2 Hz, 1H), 4.23-4.14 (m, 1H), 3.93-3.88 (m, 5H), 3.63 (d, J=10.0 Hz, 2H), 3.16-3.06 (m, 2H), 3.02 (t, J=5.6 Hz, 2H), 2.90-2.81 (m, 1H), 2.74 (s, 2H), 2.65-2.57 (m, 2H), 2.33-2.27 (m, 1H), 2.15 (dd, J=5.2, 13.2 Hz, 1H), 2.07 (d, J=9.6 Hz, 2H), 1.92-1.85 (m, 3H), 1.78 (d, J=11.6 Hz, 2H), 1.52-1.45 (m, 2H), 1.43-1.13 (m, 9H), 1.09-1.01 (m, 2H)

### Example 296. Preparation of Compound 347a

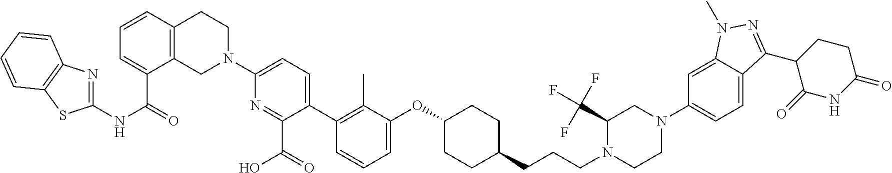 [SMILES: Cc1c(O[C@H]2CC[C@H](CCCN3CCN(c4ccc5c(C6CCC(=O)NC6=O)nn(C)c5c4)C[C@@H]3C(F)(F)F)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(3-((2R)-4-(3-(2,6-dioxopiperidin-3-yl)-1-methyl-1H-indazol-6-yl)-2-(trifluoromethyl)piperazin-1-yl)propyl)cyclohexyl)oxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(3-((2R)-4-(3-(2,6-dioxopiperidin-3-yl)-1-methyl-1H-indazol-6-yl)-2-(trifluoromethyl)piperazin-1-yl)propyl)cyclohexyl)oxy)-2-methylphenyl)picolinate

To a solution of tert-butyl 6-(8-(benzo[d]thiazol-2-ylcarbamoyl)-3,4-dihydroisoquinolin-2(1H)-yl)-3-(2-methyl-3-(((1r,4r)-4-(3-oxopropyl)cyclohexyl)oxy)phenyl)picolinate (40 mg, 54.73 mol, 1 equiv.) in DCM (2 mL) was added 3-[1-methyl-6-[(3R)-3-(trifluoromethyl)piperazin-1-yl]indazol-3-yl]piperidine-2,6-dione (25.97 mg, 65.67 μmol, 1.2 equiv.), and NaBH(OAc)₃ (57.99 mg, 273.63 μmol, 5 equiv.). The mixture was stirred at 25° C. for 2 hours. The reaction mixture was concentrated under reduced pressure to give a residue. The residue was purified by prep-TLC (SiO₂, DCM:MeOH=15:1) to give tert-butyl 6-(8-(benzo[d]thiazol-2-ylcarbamoyl)-3,4-dihydroisoquinolin-2(1H)-yl)-3-(3-(((1s,4r)-4-(3-((2R)-4-(3-(2,6-dioxopiperidin-3-yl)-1-methyl-1H-indazol-6-yl)-2-(trifluoromethyl)piperazin-1-yl)propyl)cyclohexyl)oxy)-2-methylphenyl)picolinate (40 mg, crude) as a yellow oil. MS (ESI) m/z: 1110.6[M+H]⁺.

### Step B. Procedure for Preparation of 6-(8-(benzo[d]thiazol-2-ylcarbamoyl)-3,4-dihydroisoquinolin-2(1H)-yl)-3-(3-(((1s,4r)-4-(3-((2R)-4-(3-(2,6-dioxopiperidin-3-yl)-1-methyl-1H-indazol-6-yl)-2-(trifluoromethyl)piperazin-1-yl)propyl)cyclohexyl)oxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(((1s,4r)-4-(3-((2R)-4-(3-(2,6-dioxopiperidin-3-yl)-1-methyl-1H-indazol-6-yl)-2-(trifluoromethyl)piperazin-1-yl)propyl)cyclohexyl)oxy)-2-methylphenyl)picolinate (40 mg, 36.03 mol, 1 equiv.) in DCM (1 mL) was added TFA (1 mL). The mixture was stirred at 25° C. for 16 hours. The mixture was concentrated under reduced pressure to give a residue. The residue was purified by prep-HPLC to afford 6-(8-(benzo[d]thiazol-2-ylcarbamoyl)-3,4-dihydroisoquinolin-2(1H)-yl)-3-(3-(((1s,4r)-4-(3-((2R)-4-(3-(2,6-dioxopiperidin-3-yl)-1-methyl-1H-indazol-6-yl)-2-(trifluoromethyl)piperazin-1-yl)propyl)cyclohexyl)oxy)-2-methylphenyl)picolinic acid (16.26 mg, 15.13 μmol, 41.99% yield) as an off-white solid. MS (ESI) m/z: 1054.5[M+H]⁺. 1H NMR (400 MHz, DMSO-d₆) δ=10.85 (s, 1H), 8.02 (d, J=8.0 Hz, 1H), 7.84-7.74 (m, 1H), 7.62 (d, J=7.6 Hz, 1H), 7.54-7.41 (m, 4H), 7.40-7.31 (m, 2H), 7.10-7.03 (m, 1H), 6.97-6.88 (m, 2H), 6.88-6.76 (m, 2H), 6.62 (d, J=7.6 Hz, 1H), 4.98 (s, 2H), 4.30-4.14 (m, 2H), 3.94-3.88 (m, 4H), 3.63 (d, J=12.0 Hz, 2H), 3.14-2.99 (m, 5H), 2.85 (d, J=7.6 Hz, 1H), 2.74-2.69 (m, 2H), 2.63-2.58 (m, 2H), 2.29 (dd, J=4.4, 9.6 Hz, 1H), 2.20-2.00 (m, 4H), 1.87 (s, 3H), 1.80 (d, J=12.0 Hz, 2H), 1.50 (d, J=6.0 Hz, 2H), 1.43-1.29 (m, 3H), 1.26-1.19 (m, 3H), 1.12-1.01 (m, 2H).

### Example 297. Preparation of Compound 347b

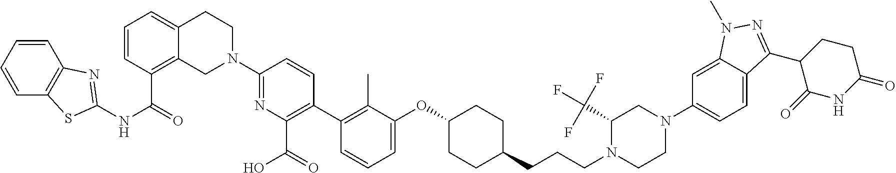 [SMILES: Cc1c(O[C@H]2CC[C@H](CCCN3CCN(c4ccc5c(C6CCC(=O)NC6=O)nn(C)c5c4)C[C@H]3C(F)(F)F)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-(3-((2S)-4-(3-(2,6-dioxopiperidin-3-yl)-1-methyl-1H-indazol-6-yl)-2-(trifluoromethyl)piperazin-1-yl)propyl)cyclohexyl)oxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(((1r,4r)-4-(3-((2S)-4-(3-(2,6-dioxopiperidin-3-yl)-1-methyl-1H-indazol-6-yl)-2-(trifluoromethyl)piperazin-1-yl)propyl)cyclohexyl)oxy)-2-methylphenyl)picolinate

To a solution of tert-butyl 6-(8-(benzo[d]thiazol-2-ylcarbamoyl)-3,4-dihydroisoquinolin-2(1H)-yl)-3-(2-methyl-3-(((1r,4r)-4-(3-oxopropyl)cyclohexyl)oxy)phenyl)picolinate (100 mg, 136.82 mol, 1.0 equiv.) and 3-(1-methyl-6-((S)-3-(trifluoromethyl)piperazin-1-yl)-1H-indazol-3-yl)piperidine-2,6-dione (64.99 mg, 150.50 μmol, 1.1 equiv., HCl) in DCM (1 mL) was added NaBH(OAc)₃ (86.99 mg, 410.45 μmol, 3.0 equiv.). The mixture was stirred at 25° C. for 2 hours. The reaction mixture was concentrated under reduced pressure to give a residue. The residue was purified by prep-TLC (SiO₂, DCM:MeOH=15:1) to give tert-butyl 6-(8-(benzo[d]thiazol-2-ylcarbamoyl)-3,4-dihydroisoquinolin-2(1H)-yl)-3-(3-(((1r,4r)-4-(3-((2S)-4-(3-(2,6-dioxopiperidin-3-yl)-1-methyl-1H-indazol-6-yl)-2-(trifluoromethyl)piperazin-1-yl)propyl)cyclohexyl)oxy)-2-methylphenyl)picolinate (74 mg, 66.65 μmol, 48.71% yield) as a yellow solid. MS (ESI) m/z: 555.9 [M/2+H]⁺.

### Step B. Procedure for Preparation of 6-(8-(benzo[d]thiazol-2-ylcarbamoyl)-3,4-dihydroisoquinolin-2(1H)-yl)-3-(3-(((1r,4r)-4-(3-((2S)-4-(3-(2,6-dioxopiperidin-3-yl)-1-methyl-1H-indazol-6-yl)-2-(trifluoromethyl)piperazin-1-yl)propyl)cyclohexyl)oxy)-2-methylphenyl)picolinic acid

A solution of tert-butyl 6-(8-(benzo[d]thiazol-2-ylcarbamoyl)-3,4-dihydroisoquinolin-2(1H)-yl)-3-(3-(((1r,4r)-4-(3-((2S)-4-(3-(2,6-dioxopiperidin-3-yl)-1-methyl-1H-indazol-6-yl)-2-(trifluoromethyl)piperazin-1-yl)propyl)cyclohexyl)oxy)-2-methylphenyl)picolinate (74 mg, 66.65 mol, 1.0 equiv.) in TFA (1 mL) and DCM (1 mL) was stirred at 25° C. for 16 hours. The reaction mixture was concentrated under reduced pressure to remove solvent to give a residue. The residue was purified by prep-HPLC to give 6-(8-(benzo[d]thiazol-2-ylcarbamoyl)-3,4-dihydroisoquinolin-2(1H)-yl)-3-(3-(((1r,4r)-4-(3-((2S)-4-(3-(2,6-dioxopiperidin-3-yl)-1-methyl-1H-indazol-6-yl)-2-(trifluoromethyl)piperazin-1-yl)propyl)cyclohexyl)oxy)-2-methylphenyl)picolinic acid (27.59 mg, 26.17 μmol, 39.27% yield) as a white solid. MS (ESI) m/z: 1054.5 [M+H]⁺. ¹H NMR (400 MHz, DMSO-d₆) δ=13.05-12.69 (m, 1H), 10.90-10.83 (m, 1H), 8.04 (d, J=7.6 Hz, 1H), 7.79 (d, J=8.4 Hz, 1H), 7.63 (d, J=7.6 Hz, 1H), 7.54-7.43 (m, 4H), 7.41-7.33 (m, 2H), 7.11-7.05 (m, 1H), 6.99-6.91 (m, 2H), 6.88-6.82 (m, 2H), 6.62 (d, J=7.2 Hz, 1H), 4.98 (s, 2H), 4.29-4.17 (m, 2H), 3.96-3.88 (m, 6H), 3.43 (m, 1H), 3.37-3.31 (m, 1H), 3.15-3.01 (m, 4H), 2.94-2.83 (m, 1H), 2.79-2.71 (m, 2H), 2.63-2.59 (m, 2H), 2.35-2.29 (m, 1H), 2.20-2.05 (m, 3H), 1.87 (s, 3H), 1.85-1.73 (m, 2H), 1.58-1.47 (m, 2H), 1.45-1.19 (m, 6H), 1.15-1.00 (m, 2H)

### Example 298. Preparation of Compound 349a

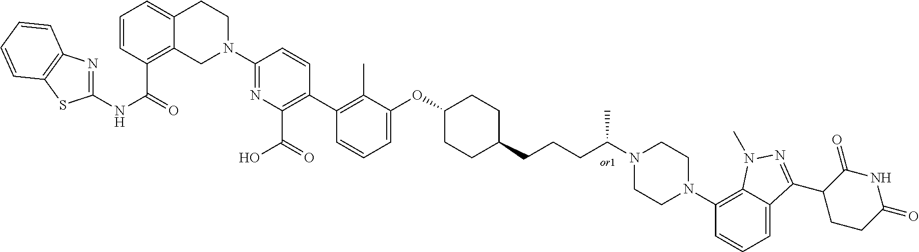 [SMILES: Cc1c(O[C@H]2CC[C@H](CCC[C@H](C)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-((4S)-4-(4-(3-(2,6-dioxopiperidin-3-yl)-1-methyl-1H-indazol-7-yl)piperazin-1-yl)pentyl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1R,4r)-4-((4S*)-4-(4-(3-(2,6-dioxopiperidin-3-yl)-1-methyl-1H-indazol-7-yl)piperazin-1-yl)pentyl)cyclohexyl)oxy)-2-methylphenyl)picolinic acid]

### Step A. Procedure for Preparation of 3-(2,6-bis(benzyloxy)pyridin-3-yl)-1-methyl-7-(4-((S)-5-((1r,4r)-4-(2-methyl-3-(4,4,5,5-tetramethyl-1,3,2-dioxaborolan-2-yl)phenoxy)cyclohexyl)pentan-2-yl)piperazin-1-yl)-1H-indazole

To a solution of 3-(2,6-bis(benzyloxy)pyridin-3-yl)-7-(4-((S)-5-((1r,4R)-4-(3-bromo-2-methylphenoxy)cyclohexyl)pentan-2-yl)piperazin-1-yl)-1-methyl-1H-indazole (200 mg, 237.28 mol, 1.0 equiv.) in dioxane (2 mL) was added Pd(dppf)Cl₂ (17.36 mg, 23.73 μmol, 0.1 equiv.), 4,4,4′,4′,5,5,5′,5′-octamethyl-2,2′-bi(1,3,2-dioxaborolane) (180.76 mg, 711.83 μmol, 3.0 equiv.), and KOAc (69.86 mg, 711.83 μmol, 3.0 equiv.). The mixture was stirred at 90° C. for 12 hours. The reaction mixture was concentrated under reduced pressure to give a residue. The residue was diluted with H₂O (50 mL) and extracted with ethyl acetate 100 mL (50 mL×2). The combined organic layers were washed with ethyl acetate 30 mL, dried over Na₂SO₄, filtered, and concentrated under reduced pressure to give a residue. The residue was purified by prep-TLC (SiO₂, petroleum ether/ethyl acetate=3/1) to give 3-(2,6-bis(benzyloxy)pyridin-3-yl)-1-methyl-7-(4-((S)-5-((1r,4r)-4-(2-methyl-3-(4,4,5,5-tetramethyl-1,3,2-dioxaborolan-2-yl)phenoxy)cyclohexyl)pentan-2-yl)piperazin-1-yl)-1H-indazole (170 mg, 181.47 μmol, 76.48% yield) as a yellow oil. MS (ESI) m/z: 890.5 [M+H]⁺.

### Step B. Procedure for Preparation of 3-(1-methyl-7-(4-((S)-5-((1r,4r)-4-(2-methyl-3-(4,4,5,5-tetramethyl-1,3,2-dioxaborolan-2-yl)phenoxy)cyclohexyl)pentan-2-yl)piperazin-1-yl)-1H-indazol-3-yl)piperidine-2,6-dione

A mixture of 3-(2,6-bis(benzyloxy)pyridin-3-yl)-1-methyl-7-(4-((S)-5-((1r,4R)-4-(2-methyl-3-(4,4,5,5-tetramethyl-1,3,2-dioxaborolan-2-yl)phenoxy)cyclohexyl)pentan-2-yl)piperazin-1-yl)-1H-indazole (170 mg, 191.02 μmol, 1.0 equiv.), Pd/C (40.66 mg, 38.20 μmol, 10% purity, 0.2 equiv.), EtOH (2 mL), and Pd(OH)₂ (26.83 mg, 38.20 μmol, 20% purity, 0.2 equiv.) in THE (2 mL) was stirred at 40° C. for 12 hours under H₂ atmosphere. The mixture was then filtered and washed with THE (50 mL). The filtrate was concentrated under reduced pressure to give 3-(1-methyl-7-(4-((S)-5-((1r,4r)-4-(2-methyl-3-(4,4,5,5-tetramethyl-1,3,2-dioxaborolan-2-yl)phenoxy)cyclohexyl)pentan-2-yl)piperazin-1-yl)-1H-indazol-3-yl)piperidine-2,6-dione (140 mg, 169.16 μmol, 88.56% yield) as a yellow solid, which was used in the next step without further purification. MS (ESI) m/z: 712.7 [M+H]⁺. ¹H NMR (400 MHz, DMSO-d₆) δ=10.89 (s, 1H), 7.44-7.34 (m, 1H), 7.21-7.13 (m, 1H), 7.11-7.01 (m, 4H), 5.76 (s, 1H), 4.34 (dd, J=4.8, 9.2 Hz, 1H), 4.28-4.11 (m, 5H), 3.93 (s, 1H), 2.90 (s, 2H), 2.74 (s, 2H), 2.69-2.60 (m, 4H), 2.38-2.31 (m, 2H), 2.30 (s, 3H), 2.19 (s, 3H), 2.08-2.01 (m, 2H), 1.82-1.75 (m, 2H), 1.56-1.44 (m, 2H), 1.26-1.19 (m, 5H), 1.17 (s, 1H), 1.09-1.01 (m, 12H), 0.99 (d, J=6.4 Hz, 3H)

### Step C. Procedure for Preparation of tert-butyl 6-(8-(benzo[d]thiazol-2-ylcarbamoyl)-3,4-dihydroisoquinolin-2(1H)-yl)-3-(3-(((1r,4r)-4-((4S)-4-(4-(3-(2,6-dioxopiperidin-3-yl)-1-methyl-1H-indazol-7-yl)piperazin-1-yl)pentyl)cyclohexyl)oxy)-2-methylphenyl)picolinate

A mixture of 3-(1-methyl-7-(4-((S)-5-((1r,4R)-4-(2-methyl-3-(4,4,5,5-tetramethyl-1,3,2-dioxaborolan-2-yl)phenoxy)cyclohexyl)pentan-2-yl)piperazin-1-yl)-1H-indazol-3-yl)piperidine-2,6-dione (140 mg, 196.70 μmol, 1.0 equiv.), tert-butyl 6-(8-(benzo[d]thiazol-2-ylcarbamoyl)-3,4-dihydroisoquinolin-2(1H)-yl)-3-bromopicolinate (133.48 mg, 236.04 μmol, 1.2 equiv.), KF (34.29 mg, 590.10 μmol, 13.82 μL, 3.0 equiv.), [2-(2-aminophenyl)phenyl]palladium(1⁺)bis(1-adamantyl)-butyl-phosphanemethanesulfonate (28.65 mg, 39.34 μmol, 0.2 equiv.), and H₂O (0.2 mL) in dioxane (2 mL) was degassed and purged with N₂ three times. The mixture was stirred at 100° C. for 1 hour under N₂ atmosphere. The reaction mixture was concentrated under reduced pressure to remove solvent. The residue was diluted with H₂O (30 mL) and extracted with ethyl acetate 100 mL (50 mL×2). The combined organic layers were washed with brine 30 mL, dried over Na₂SO₄, filtered and concentrated under reduced pressure to give a residue. The residue was purified by prep-TLC (SiO₂, DCM:MeOH=20:1) to give tert-butyl 6-(8-(benzo[d]thiazol-2-ylcarbamoyl)-3,4-dihydroisoquinolin-2(1H)-yl)-3-(3-(((1r,4r)-4-((4S)-4-(4-(3-(2,6-dioxopiperidin-3-yl)-1-methyl-1H-indazol-7-yl)piperazin-1-yl)pentyl)cyclohexyl)oxy)-2-methylphenyl)picolinate (80 mg, 66.52 μmol, 33.82% yield) as a yellow oil. MS (ESI) m/z: 1070.6 [M+H]⁺.

### Step D. Procedure for Preparation of 6-(8-(benzo[d]thiazol-2-ylcarbamoyl)-3,4-dihydroisoquinolin-2(1H)-yl)-3-(3-(((1r,4r)-4-((4S)-4-(4-(3-(2,6-dioxopiperidin-3-yl)-1-methyl-1H-indazol-7-yl)piperazin-1-yl)pentyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1R,4r)-4-((4S*)-4-(4-(3-(2,6-dioxopiperidin-3-yl)-1-methyl-1H-indazol-7-yl)piperazin-1-yl)pentyl)cyclohexyl)oxy)-2-methylphenyl)picolinic acid]

To a solution of tert-butyl 6-(8-(benzo[d]thiazol-2-ylcarbamoyl)-3,4-dihydroisoquinolin-2(1H)-yl)-3-(3-(((1r,4r)-4-((4S)-4-(4-(3-(2,6-dioxopiperidin-3-yl)-1-methyl-1H-indazol-7-yl)piperazin-1-yl)pentyl)cyclohexyl)oxy)-2-methylphenyl)picolinate (80 mg, 74.74 μmol, 1.0 equiv.) in DCM (0.5 mL) was added TFA (1 mL). The mixture was stirred at 40° C. for 1 hour. The reaction mixture was concentrated under reduced pressure to remove solvent. The residue was purified by prep-HPLC to give 6-(8-(benzo[d]thiazol-2-ylcarbamoyl)-3,4-dihydroisoquinolin-2(1H)-yl)-3-(3-(((1r,4r)-4-((4S)-4-(4-(3-(2,6-dioxopiperidin-3-yl)-1-methyl-1H-indazol-7-yl)piperazin-1-yl)pentyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1R,4r)-4-((4S*)-4-(4-(3-(2,6-dioxopiperidin-3-yl)-1-methyl-1H-indazol-7-yl)piperazin-1-yl)pentyl)cyclohexyl)oxy)-2-methylphenyl)picolinic acid](29.37 mg, 28.38 μmol, 37.97% yield) as a white solid. MS (ESI) m/z: 1014.6 [M+H]⁺. ¹H NMR (400 MHz, DMSO-d₆) δ=12.96-12.77 (m, 1H), 12.73-12.41 (m, 1H), 10.89 (s, 1H), 8.04 (d, J=7.2 Hz, 1H), 7.80 (d, J=8.0 Hz, 1H), 7.63 (d, J=7.6 Hz, 1H), 7.50-7.34 (m, 6H), 7.10-6.91 (m, 5H), 6.62 (d, J=7.6 Hz, 1H), 4.98 (s, 2H), 4.38-4.32 (m, 1H), 4.25 (s, 3H), 4.22-4.16 (m, 1H), 3.92 (s, 2H), 3.03 (s, 2H), 2.73-2.55 (m, 5H), 2.44-2.29 (m, 4H), 2.22-2.01 (m, 4H), 1.93 (s, 1H), 1.88 (s, 3H), 1.84-1.71 (m, 3H), 1.64-1.50 (m, 1H), 1.44-1.20 (m, 10H), 1.13-1.05 (m, 3H).

### Example 299. Preparation of Compound 349b

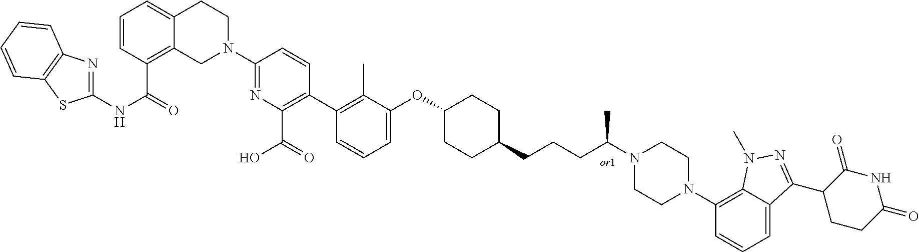 [SMILES: Cc1c(O[C@H]2CC[C@H](CCC[C@@H](C)N3CCN(c4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1S,4r)-4-((4R)-4-(4-(3-(2,6-dioxopiperidin-3-yl)-1-methyl-1H-indazol-7-yl)piperazin-1-yl)pentyl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1S,4r)-4-((4R*)-4-(4-(3-(2,6-dioxopiperidin-3-yl)-1-methyl-1H-indazol-7-yl)piperazin-1-yl)pentyl)cyclohexyl)oxy)-2-methylphenyl)picolinic acid]

### Step A. Procedure for Preparation of 3-(2,6-dibenzyloxy-3-pyridyl)-1-methyl-7-piperazin-1-yl-indazole

A mixture of 7-bromo-3-(2,6-dibenzyloxy-3-pyridyl)-1-methyl-indazole (2.5 g, 5.00 mmol, 1.0 equiv.), piperazine (1.29 g, 14.99 mmol, 3.0 equiv.), Pd₂(dba)₃ (457.51 mg, 499.61 μmol, 0.1 equiv.), NaOtBu (960.26 mg, 9.99 mmol, 2.0 equiv.), and tritert-butylphosphane (2.02 g, 999.23 mol, 2.35 mL, 10% purity, 0.2 equiv.) in toluene (100 mL) was degassed and purged with N₂ three times. The mixture was stirred at 100° C. for 4 hours under N₂ atmosphere. The reaction mixture was concentrated under reduced pressure to give a residue. The residue was purified by flash silica gel chromatography (10˜30% MeOH/DCM) to give 3-(2,6-dibenzyloxy-3-pyridyl)-1-methyl-7-piperazin-1-yl-indazole (1.5 g, 2.88 mmol, 57.64% yield) as a yellow oil. MS (ESI)m/z: 506.4 [M+H]⁺.

### Step B. Procedure for Preparation of 3-(2,6-bis(benzyloxy)pyridin-3-yl)-7-(4-(5-((1r,4s)-4-(3-bromo-2-methylphenoxy) cyclohexyl)pentan-2-yl)piperazin-1-yl)-1-methyl-1H-indazole

To a solution of 5-((1r,4s)-4-(3-bromo-2-methylphenoxy)cyclohexyl)pentan-2-one (1.5 g, 4.25 mmol, 1.0 equiv.) in DCM (30 mL) was added NaBH(OAc)₃ (3.60 g, 16.98 mmol, 4.0 equiv.) and 3-(2,6-dibenzyloxy-3-pyridyl)-1-methyl-7-piperazin-1-yl-indazole (2.15 g, 4.25 mmol, 1.0 equiv.). The mixture was stirred at 25° C. for 12 hours. Then the reaction mixture was concentrated under reduced pressure to give a residue. The residue was diluted with H₂O (100 mL) and extracted with DCM (50 mL×3). The combined organic layers were washed with DCM (50 mL), dried over Na₂SO₄, filtered and concentrated under reduced pressure to give a residue. The residue was purified by flash silica gel chromatography (0˜28% ethyl acetate/petroleum ether) to give 3-(2,6-bis(benzyloxy)pyridin-3-yl)-7-(4-(5-((1r,4s)-4-(3-bromo-2-methylphenoxy) cyclohexyl)pentan-2-yl)piperazin-1-yl)-1-methyl-1H-indazole (800 mg, 892.16 μmol, 21.01% yield) as a yellow solid. MS (ESI) m/z: 844.3 [M+H]⁺.

### Step C. Procedure for Preparation of 3-(2,6-bis(benzyloxy)pyridin-3-yl)-7-(4-((S)-5-((1r,4r)-4-(3-bromo-2-methylphenoxy)cyclohexyl)pentan-2-yl)piperazin-1-yl)-1-methyl-1H-indazole and 3-(2,6-bis(benzyloxy)pyridin-3-yl)-7-(4-((R)-5-((1r,4s)-4-(3-bromo-2-methylphenoxy)cyclohexyl)pentan-2-yl)piperazin-1-yl)-1-methyl-1H-indazole

The stereoisomers of 3-(2,6-bis(benzyloxy)pyridin-3-yl)-7-(4-(5-((1r,4s)-4-(3-bromo-2-methylphenoxy) cyclohexyl)pentan-2-yl)piperazin-1-yl)-1-methyl-1H-indazole (800 mg, 892.16 mol, 1.0 equiv.) were separated by SFC to give 3-(2,6-bis(benzyloxy)pyridin-3-yl)-7-(4-((S)-5-((1r,4r)-4-(3-bromo-2-methylphenoxy)cyclohexyl)pentan-2-yl)piperazin-1-yl)-1-methyl-1H-indazole (450 mg, 533.87 μmol, 56.25% yield) as a yellow solid and 3-(2,6-bis(benzyloxy)pyridin-3-yl)-7-(4-((R)-5-((1r,4s)-4-(3-bromo-2-methylphenoxy)cyclohexyl)pentan-2-yl)piperazin-1-yl)-1-methyl-1H-indazole (300 mg, 336.34 μmol, 35.44% yield) as a yellow solid.

### Step D. Procedure for Preparation of 3-(2,6-bis(benzyloxy)pyridin-3-yl)-1-methyl-7-(4-((R)-5-((1r,4s)-4-(2-methyl-3-(4,4,5,5-tetramethyl-1,3,2-dioxaborolan-2-yl)phenoxy)cyclohexyl)pentan-2-yl)piperazin-1-yl)-1H-indazole

A mixture of 3-(2,6-bis(benzyloxy)pyridin-3-yl)-7-(4-((R)-5-((1r,4S)-4-(3-bromo-2-methylphenoxy)cyclohexyl)pentan-2-yl)piperazin-1-yl)-1-methyl-1H-indazole (300 mg, 355.91 mol, 1.0 equiv.), 4,4,4′,4′,5,5,5′,5′-octamethyl-2,2′-bi(1,3,2-dioxaborolane) (271.14 mg, 1.07 mmol, 3.0 equiv.), KOAc (104.79 mg, 1.07 mmol, 3.0 equiv.), and Pd(dppf)Cl₂ (52.08 mg, 71.18 mol, 0.2 equiv.) in dioxane (3 mL) was degassed and purged with N₂ three times. The mixture was stirred at 90° C. for 12 hours under N₂ atmosphere. To this solution, ethyl acetate (100 mL) and water (100 mL) were added, and the layers were separated. The aqueous was extracted with ethyl acetate (30 mL×2), dried over anhydrous sodium sulfate, filtered, and concentrated under vacuum. The residue was purified by prep-TLC (SiO₂, petroleum ether:ethyl acetate=3:1) to give 3-(2,6-bis(benzyloxy)pyridin-3-yl)-1-methyl-7-(4-((R)-5-((1r,4s)-4-(2-methyl-3-(4,4,5,5-tetramethyl-1,3,2-dioxaborolan-2-yl)phenoxy)cyclohexyl)pentan-2-yl)piperazin-1-yl)-1H-indazole (300 mg, 323.17 μmol, 90.80% yield) as a yellow solid. MS (ESI) m/z: 890.8 [M+H]⁺

### Step E. Procedure for Preparation of 3-(1-methyl-7-(4-((R)-5-((1r,4s)-4-(2-methyl-3-(4,4,5,5-tetramethyl-1,3,2-dioxaborolan-2-yl)phenoxy)cyclohexyl)pentan-2-yl)piperazin-1-yl)-1H-indazol-3-yl)piperidine-2,6-dione

A mixture of 3-(2,6-bis(benzyloxy)pyridin-3-yl)-1-methyl-7-(4-((R)-5-((1r,4s)-4-(2-methyl-3-(4,4,5,5-tetramethyl-1,3,2-dioxaborolan-2-yl)phenoxy)cyclohexyl)pentan-2-yl)piperazin-1-yl)-1H-indazole (300 mg, 337.09 μmol, 1.0 equiv.) in THE (3 mL) and EtOH (3 mL) was added Pd/C (100 mg, 93.97 μmol, 10% purity) and Pd(OH)₂ (100 mg, 142.41 μmol, 20% purity). The mixture was stirred at 40° C. for 16 hours under H₂ atmosphere (15 Psi). The reaction mixture was filtered and concentrated under reduced pressure to give a filter cake with a little EtOH. The collected filtrate was concentrated under reduced pressure to give 3-(1-methyl-7-(4-((R)-5-((1r,4s)-4-(2-methyl-3-(4,4,5,5-tetramethyl-1,3,2-dioxaborolan-2-yl)phenoxy)cyclohexyl)pentan-2-yl)piperazin-1-yl)-1H-indazol-3-yl)piperidine-2,6-dione (250 mg, 333.69 μmol, 98.99% yield) as a pink solid. ¹H NMR (400 MHz, DMSO) δ=10.88 (s, 1H), 7.39-7.37 (m, 1H), 7.18-7.16 (m, 1H), 7.12-7.08 (m, 1H), 7.08-7.06 (m, 1H), 7.04-6.98 (m, 2H), 4.37-4.30 (m, 1H), 4.24 (s, 3H), 4.20-4.11 (m, 1H), 3.20-2.98 (m, 2H), 2.83-2.55 (m, 8H), 2.38-2.30 (m, 1H), 2.29 (s, 3H), 2.20-2.12 (m, 1H), 2.07-1.98 (m, 2H), 1.83-1.72 (m, 2H), 1.55-1.45 (m, 1H), 1.38-1.32 (m, 4H), 1.29 (s, 12H), 1.27-1.18 (m, 5H), 1.03 (d, J=13.2 Hz, 2H), 0.98 (d, J=6.4 Hz, 3H)

### Step F. Procedure for Preparation of tert-butyl 6-(8-(benzo[d]thiazol-2-ylcarbamoyl)-3,4-dihydroisoquinolin-2(1H)-yl)-3-(3-(((1s,4r)-4-((4R)-4-(4-(3-(2,6-dioxopiperidin-3-yl)-1-methyl-1H-indazol-7-yl)piperazin-1-yl)pentyl)cyclohexyl)oxy)-2-methylphenyl)picolinate

A mixture of 3-(1-methyl-7-(4-((R)-5-((1r,4s)-4-(2-methyl-3-(4,4,5,5-tetramethyl-1,3,2-dioxaborolan-2-yl)phenoxy)cyclohexyl)pentan-2-yl)piperazin-1-yl)-1H-indazol-3-yl)piperidine-2,6-dione (143.01 mg, 252.90 μmol, 1.2 equiv.), KF (36.73 mg, 632.25 μmol, 14.81 μL, 3.0 equiv.), and H₂O (0.4 mL) in dioxane (4 mL) was degassed and purged with N₂ three times. To this mixture was added [2-(2-aminophenyl)phenyl]palladium(1⁺); bis(1-adamantyl)-butyl-phosphane; methanesulfonate (30.70 mg, 42.15 μmol, 0.2 equiv.). The resulting mixture was stirred at 100° C. for 1 hour under N₂ atmosphere. Ethyl acetate (100 mL) and water (100 mL) were added, and the layers were separated. The organic layers were washed with brine (30 mL×2), dried over anhydrous sodium sulfate, filtered, and concentrated under vacuum. The residue was purified by prep-TLC (SiO₂, DCM:MeOH=20:1) to give tert-butyl 6-(8-(benzo[d]thiazol-2-ylcarbamoyl)-3,4-dihydroisoquinolin-2(1H)-yl)-3-(3-(((1s,4r)-4-((4R)-4-(4-(3-(2,6-dioxopiperidin-3-yl)-1-methyl-1H-indazol-7-yl)piperazin-1-yl)pentyl)cyclohexyl)oxy)-2-methylphenyl)picolinate (120 mg, 98.66 μmol, 46.81% yield) as a yellow solid. MS (ESI) m/z: 536.2 [½M+H]⁺.

### Step G. Procedure for Preparation of 6-(8-(benzo[d]thiazol-2-ylcarbamoyl)-3,4-dihydroisoquinolin-2(1H)-yl)-3-(3-(((1S,4r)-4-((4R)-4-(4-(3-(2,6-dioxopiperidin-3-yl)-1-methyl-1H-indazol-7-yl)piperazin-1-yl)pentyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1S,4r)-4-((4R*)-4-(4-(3-(2,6-dioxopiperidin-3-yl)-1-methyl-1H-indazol-7-yl)piperazin-1-yl)pentyl)cyclohexyl)oxy)-2-methylphenyl)picolinic acid]

A solution of tert-butyl 6-(8-(benzo[d]thiazol-2-ylcarbamoyl)-3,4-dihydroisoquinolin-2(1H)-yl)-3-(3-(((1s,4r)-4-((4R)-4-(4-(3-(2,6-dioxopiperidin-3-yl)-1-methyl-1H-indazol-7-yl)piperazin-1-yl)pentyl)cyclohexyl)oxy)-2-methylphenyl)picolinate (120 mg, 112.11 μmol, 1.0 equiv.) in TFA (2 mL) and DCM (1 mL) was stirred at 40° C. for 1 hour. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1S,4r)-4-((4R)-4-(4-(3-(2,6-dioxopiperidin-3-yl)-1-methyl-1H-indazol-7-yl)piperazin-1-yl)pentyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1S,4r)-4-((4R*)-4-(4-(3-(2,6-dioxopiperidin-3-yl)-1-methyl-1H-indazol-7-yl)piperazin-1-yl)pentyl)cyclohexyl)oxy)-2-methylphenyl)picolinic acid] (39.51 mg, 37.88 μmol, 33.78% yield) as a white solid. MS (ESI) m/z: 1014.5 [M+H]⁺. ¹H NMR (400 MHz, DMSO) δ=12.90-12.81 (m, 1H), 10.88 (s, 1H), 8.03 (d, J=8.0 Hz, 1H), 7.79 (d, J=8.0 Hz, 1H), 7.62 (d, J=7.6 Hz, 1H), 7.49-7.33 (m, 6H), 7.10-7.02 (m, 3H), 6.97-6.91 (m, 2H), 6.61 (d, J=7.6 Hz, 1H), 4.98 (s, 2H), 4.38-4.31 (m, 1H), 4.24 (s, 3H), 4.22-4.14 (m, 1H), 3.91 (t, J=6.0 Hz, 2H), 3.24-3.13 (m, 2H), 3.02 (t, J=5.6 Hz, 2H), 2.96-2.73 (m, 4H), 2.70-2.59 (m, 3H), 2.41-2.25 (m, 2H), 2.21-2.14 (m, 1H), 2.12-2.05 (m, 2H), 1.87 (s, 3H), 1.82-1.76 (m, 2H), 1.66-1.51 (m, 1H), 1.47-1.17 (m, 10H), 1.11-1.00 (m, 4H)

### Example 300. Preparation of Compound 350a

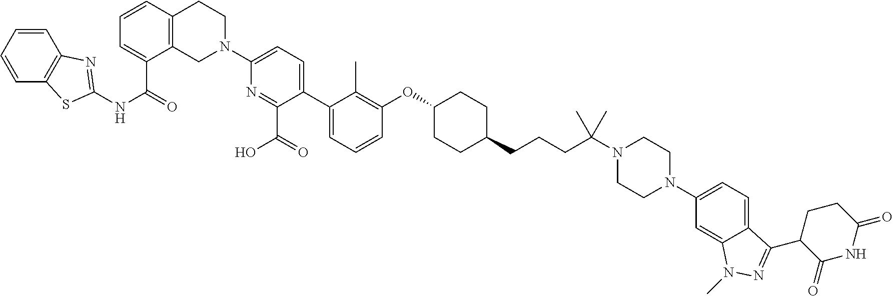 [SMILES: Cc1c(O[C@H]2CC[C@H](CCCC(C)(C)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)-4-methylpentyl)cyclohexyl)oxy)-2-methylphenyl)picolinic acid

### Step A. Procedure for Preparation of 4-((1r,4s)-4-(3-bromo-2-methylphenoxy)cyclohexyl)butanoic acid

To a solution of 4-((1r,4s)-4-(3-bromo-2-methylphenoxy)cyclohexyl)butanal (670 mg, 1.97 mmol, 1.0 equiv.) in THF (6 mL), was added t-BuOH (10 mL) and H₂O (3 mL), NaH₂PO₄ (710.81 mg, 5.92 mmol, 3.0 equiv.), 2-methylbut-2-ene (831.01 mg, 11.85 mmol, 1.26 mL, 6.0 equiv.) and NaClO₂ (535.83 mg, 5.92 mmol, 3.0 equiv.). The mixture was stirred at 20° C. for 16 hours under O₂ (15 Psi). The reaction mixture was filtered and concentrated under reduced pressure to give a residue. The residue was purified by flash silica gel chromatography (0-6% ethyl acetate/petroleum ether) to give 4-((1r,4s)-4-(3-bromo-2-methylphenoxy)cyclohexyl)butanoic acid (560 mg, crude) as a yellow oil. ¹H NMR (400 MHz, DMSO-d₆) δ=11.97 (s, 1H), 7.16-7.12 (m, 1H), 7.09-7.04 (m, 1H), 7.03-6.99 (m, 1H), 4.27-4.18 (m, 1H), 2.24-2.13 (m, 5H), 2.08-2.00 (m, 2H), 1.76 (d, J=11.2 Hz, 2H), 1.56-1.46 (m, 2H), 1.39-1.29 (m, 2H), 1.28-1.23 (m, 1H), 1.21-1.15 (m, 2H), 1.09-0.97 (m, 2H)

### Step B. Procedure for Preparation of 1-(4-(3-(2,6-bis(benzyloxy)pyridin-3-yl)-1-methyl-1H-indazol-6-yl)piperazin-1-yl)-4-((1r,4s)-4-(3-bromo-2-methylphenoxy)cyclohexyl)butan-1-one

To a solution of 4-((1r,4s)-4-(3-bromo-2-methylphenoxy)cyclohexyl)butanoic acid (560 mg, 1.58 mmol, 1 equiv.) in DMF (6 mL) was added HATU (719.22 mg, 1.89 mmol, 1.2 equiv.), TEA (478.51 mg, 4.73 mmol, 658.20 μL, 3.0 equiv.), and 3-(2,6-bis(benzyloxy)pyridin-3-yl)-1-methyl-6-(piperazin-1-yl)-1H-indazole (916.53 mg, 1.81 mmol, 1.15 equiv.). The mixture was stirred at 20° C. for 0.5 hours. The mixture was poured into H₂O (20 mL), filtered, and concentrated under reduced pressure to give a residue. The residue was purified by flash silica gel chromatography (0˜50% ethyl acetate/petroleum ether) to give 1-(4-(3-(2,6-bis(benzyloxy)pyridin-3-yl)-1-methyl-1H-indazol-6-yl)piperazin-1-yl)-4-((1r,4s)-4-(3-bromo-2-methylphenoxy)cyclohexyl)butan-1-one (1.07 g, 1.19 mmol, 75.54% yield) as a yellow oil. MS (ESI) m/z: 844.0 [M+H]⁺. ¹H NMR (400 MHz, DMSO-d₆) δ=7.89 (d, J=8.0 Hz, 1H), 7.53 (d, J=8.8 Hz, 1H), 7.48-7.44 (m, 2H), 7.42-7.24 (m, 8H), 7.15-7.11 (m, 1H), 7.09-7.04 (m, 1H), 7.03-6.98 (m, 1H), 6.88 (s, 1H), 6.84 (d, J=9.2 Hz, 1H), 6.56 (d, J=8.0 Hz, 1H), 5.43 (d, J=14.0 Hz, 4H), 4.28-4.18 (m, 1H), 3.97 (s, 3H), 3.62 (s, 4H), 3.22 (s, 2H), 3.17 (s, 2H), 2.37-2.32 (m, 2H), 2.20 (s, 3H), 2.08-2.00 (m, 2H), 1.77 (d, J=12.0 Hz, 2H), 1.59-1.49 (m, 2H), 1.41-1.30 (m, 2H), 1.30-1.26 (m, 1H), 1.25-1.20 (m, 2H), 1.09-0.98 (m, 2H)

### Step C. Procedure for Preparation of 3-(2,6-bis(benzyloxy)pyridin-3-yl)-6-(4-(5-((1r,4s)-4-(3-bromo-2-methylphenoxy)cyclohexyl)-2-methylpentan-2-yl)piperazin-1-yl)-1-methyl-1H-indazole

A mixture of 1-(4-(3-(2,6-bis(benzyloxy)pyridin-3-yl)-1-methyl-1H-indazol-6-yl)piperazin-1-yl)-4-((1r,4s)-4-(3-bromo-2-methylphenoxy)cyclohexyl)butan-1-one (500 mg, 593.22 μmol, 1.0 equiv.), and ZrCl₄ (165.89 mg, 711.86 μmol, 59.25 μL, 1.2 equiv.) in THE (10 mL) was degassed and purged with N₂ three times. To this solution, MeMgBr (2.5 M, 1.66 mL, 7.0 equiv.) was added dropwise at −10° C. The resulting mixture was stirred at −10° C. for 15 minutes under N₂ atmosphere. The reaction mixture was poured into saturated NH₄Cl aq. (40 mL), and then extracted was ethyl acetate 120 mL (40 mL×3). The combined organic layers were washed with brine 50 mL, dried over Na₂SO₄, filtered, and concentrated under reduced pressure to give a residue. The residue was purified by flash silica gel chromatography (0˜50% ethyl acetate/petroleum ether) to give 3-(2,6-bis(benzyloxy)pyridin-3-yl)-6-(4-(5-((1r,4s)-4-(3-bromo-2-methylphenoxy)cyclohexyl)-2-methylpentan-2-yl)piperazin-1-yl)-1-methyl-1H-indazole (60 mg, 65.29 μmol, 11.01% yield) as a yellow oil. MS (ESI) m/z: 858.2 [M+H]⁺. ¹H NMR (400 MHz, DMSO-d₆) δ=7.88 (d, J=8.4 Hz, 1H), 7.52-7.43 (m, 3H), 7.42-7.23 (m, 8H), 7.15-7.11 (m, 1H), 7.09-7.04 (m, 1H), 7.03-6.99 (m, 1H), 6.84-6.77 (m, 2H), 6.56 (d, J=8.0 Hz, 1H), 5.44 (s, 2H), 5.41 (s, 2H), 4.27-4.18 (m, 1H), 3.96 (s, 3H), 3.20-3.14 (m, 4H), 2.63 (s, 4H), 2.20 (s, 3H), 2.05 (d, J=12.8 Hz, 2H), 1.78 (d, J=10.8 Hz, 2H), 1.41-1.30 (m, 6H), 1.23 (s, 1H), 1.20-1.17 (m, 2H), 1.09-1.02 (m, 1H), 1.00 (s, 6H)

### Step D. Procedure for Preparation of 3-(2,6-bis(benzyloxy)pyridin-3-yl)-1-methyl-6-(4-(2-methyl-5-((1r,4s)-4-(2-methyl-3-(4,4,5,5-tetramethyl-1,3,2-dioxaborolan-2-yl)phenoxy)cyclohexyl)pentan-2-yl)piperazin-1-yl)-1H-indazole

A mixture of 3-(2,6-bis(benzyloxy)pyridin-3-yl)-6-(4-(5-((1r,4s)-4-(3-bromo-2-methylphenoxy)cyclohexyl)-2-methylpentan-2-yl)piperazin-1-yl)-1-methyl-1H-indazole (60 mg, 70.02 μmol, 1.0 equiv.), 4,4,4′,4′,5,5,5′,5′-octamethyl-2,2′-bi(1,3,2-dioxaborolane) (26.67 mg, 105.03 μmol, 1.5 equiv.), KOAc (34.36 mg, 350.09 μmol, 5.0 equiv.), and Pd(dppf)Cl₂ (10.25 mg, 14.00 μmol, 0.2 equiv.) in dioxane (1 mL) was degassed and purged with N₂ three times. The mixture was stirred at 100° C. for 16 hours under N₂ atmosphere. The mixture was concentrated under reduced pressure to give a residue. The residue was purified by prep-TLC (SiO₂, ethyl acetate:methanol=20:1) to give 3-(2,6-bis(benzyloxy)pyridin-3-yl)-1-methyl-6-(4-(2-methyl-5-((1r,4s)-4-(2-methyl-3-(4,4,5,5-tetramethyl-1,3,2-dioxaborolan-2-yl)phenoxy)cyclohexyl)pentan-2-yl)piperazin-1-yl)-1H-indazole (50 mg, 46.24 μmol, 66.05% yield) as a yellow oil. MS (ESI) m/z: 904.5 [M+H]⁺. ¹H NMR (400 MHz, DMSO-d₆) δ=7.88 (d, J=8.0 Hz, 1H), 7.51-7.44 (m, 3H), 7.42-7.33 (m, 5H), 7.32-7.26 (m, 3H), 7.19-7.15 (m, 1H), 7.12-7.04 (m, 2H), 6.83-6.79 (m, 2H), 6.56 (d, J=8.0 Hz, 1H), 5.44 (s, 2H), 5.41 (s, 2H), 4.22-4.13 (m, 1H), 3.96 (s, 3H), 3.17 (s, 2H), 3.16 (s, 2H), 2.63 (s, 4H), 2.29 (s, 3H), 2.07-1.99 (m, 2H), 1.81-1.74 (m, 2H), 1.41-1.31 (m, 6H), 1.28 (s, 11H), 1.23 (s, 1H), 1.20-1.16 (m, 2H), 1.08-1.02 (m, 2H), 1.00 (s, 6H)

### Step E. Procedure for Preparation of 3-(1-methyl-6-(4-(2-methyl-5-((1r,4s)-4-(2-methyl-3-(4,4,5,5-tetramethyl-1,3,2-dioxaborolan-2-yl)phenoxy)cyclohexyl)pentan-2-yl)piperazin-1-yl)-1H-indazol-3-yl)piperidine-2,6-dione

To a solution of 3-(2,6-bis(benzyloxy)pyridin-3-yl)-1-methyl-6-(4-(2-methyl-5-((1r,4s)-4-(2-methyl-3-(4,4,5,5-tetramethyl-1,3,2-dioxaborolan-2-yl)phenoxy)cyclohexyl)pentan-2-yl)piperazin-1-yl)-1H-indazole (50 mg, 55.31 μmol, 1.0 equiv.) in EtOH (2 mL) and THF (2 mL) was added Pd/C (25.31 mg, 23.78 μmol, 10% purity, 0.43 equiv.) and Pd(OH)₂ (25.24 mg, 35.95 mol, 20% purity, 0.65 equiv.) under N₂ atmosphere. The suspension was degassed and purged with H₂ three times. The mixture was stirred under H₂ (15 psi) at 40° C. for 16 hours. The reaction mixture was filtered and concentrated under reduced pressure to give a residue. The residue was purified by prep-TLC (SiO₂, ethyl acetate:methanol=20:1) to give 3-(1-methyl-6-(4-(2-methyl-5-((1r,4s)-4-(2-methyl-3-(4,4,5,5-tetramethyl-1,3,2-dioxaborolan-2-yl)phenoxy)cyclohexyl)pentan-2-yl)piperazin-1-yl)-1H-indazol-3-yl)piperidine-2,6-dione (35 mg, 41.26 μmol, 74.60% yield) as a yellow oil. MS (ESI) m/z: 726.4 [M+H]⁺

### Step F. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)-4-methylpentyl)cyclohexyl)oxy)-2-methylphenyl)picolinate

A mixture of 3-(1-methyl-6-(4-(2-methyl-5-((1r,4s)-4-(2-methyl-3-(4,4,5,5-tetramethyl-1,3,2-dioxaborolan-2-yl)phenoxy)cyclohexyl)pentan-2-yl)piperazin-1-yl)-1H-indazol-3-yl)piperidine-2,6-dione (35 mg, 48.22 μmol, 1.0 equiv.), tert-butyl 6-(8-(benzo[d]thiazol-2-ylcarbamoyl)-3,4-dihydroisoquinolin-2(1H)-yl)-3-bromopicolinate (30.00 mg, 53.05 μmol, 1.1 equiv.), and KF (8.41 mg, 144.67 μmol, 3.39 μL, 3.0 equiv.) in dioxane (1 mL) and H₂O (0.1 mL) was degassed and purged with N₂ three times. To this solution, Ad₂nBuP Pd G₃ (7.02 mg, 9.64 μmol, 0.2 equiv.) was added, and the mixture was stirred at 100° C. for 2 hours under N₂ atmosphere. The mixture was concentrated under reduced pressure to give a residue. The residue was purified by prep-TLC (SiO₂, DCM:MeOH=10:1) to give tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)-4-methylpentyl)cyclohexyl)oxy)-2-methylphenyl)picolinate (20 mg, 15.21 μmol, 31.54% yield) as a yellow solid. MS (ESI) m/z: 1084.8 [M+H]⁺

### Step G. Procedure for Preparation of 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)-4-methylpentyl)cyclohexyl)oxy)-2-methylphenyl)picolinic acid

To a solution of tert-butyl 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)-4-methylpentyl)cyclohexyl)oxy)-2-methylphenyl)picolinate (20 mg, 18.44 μmol, 1.0 equiv.) in DCM (0.5 mL) was added TFA (767.50 mg, 6.73 mmol, 0.5 mL, 364.95 equiv.). The mixture was stirred at 25° C. for 16 hours. The mixture was concentrated under reduced pressure to give a residue, which was diluted with DCM (2 mL) and adjusted to pH=7 with triethylamine. This resulting solution was concentrated to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-(4-(4-(3-(2,6-dioxopiperidin-3-yl)-1-methyl-1H-indazol-6-yl)piperazin-1-yl)-4-methylpentyl)cyclohexyl)oxy)-2-methylphenyl)picolinic acid (17.26 mg, 16.50 μmol, 89.45% yield) as a yellow solid. MS (ESI) m/z: 1028.5 [M+H]⁺. ¹H NMR (400 MHz, DMSO-d₆) δ=12.91-12.49 (m, 1H), 10.84 (s, 1H), 8.03 (d, J=8.0 Hz, 1H), 7.78 (d, J=8.4 Hz, 1H), 7.62 (d, J=7.2 Hz, 1H), 7.51-7.40 (m, 4H), 7.40-7.31 (m, 2H), 7.09-7.03 (m, 1H), 6.97-6.87 (m, 3H), 6.82 (s, 1H), 6.61 (d, J=7.6 Hz, 1H), 4.97 (s, 2H), 4.28-4.22 (m, 1H), 4.22-4.14 (m, 1H), 3.96-3.89 (m, 2H), 3.88 (s, 3H), 3.18 (s, 4H), 3.02 (t, J=5.6 Hz, 2H), 2.70-2.60 (m, 6H), 2.31-2.24 (m, 1H), 2.18-2.12 (m, 1H), 2.11-2.04 (m, 2H), 1.86 (s, 3H), 1.81-1.74 (m, 2H), 1.42-1.29 (m, 7H), 1.10-1.03 (m, 2H), 1.09-1.03 (m, 2H), 1.00 (s, 6H)

### Example 301. Preparation of Compound 334a

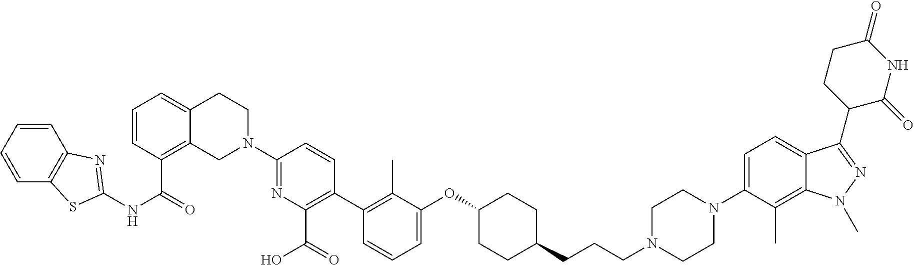 [SMILES: Cc1c(O[C@H]2CC[C@H](CCCN3CCN(c4ccc5c(C6CCC(=O)NC6=O)nn(C)c5c4C)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1,7-dimethyl-1H-indazol-6-yl)piperazin-1-yl)propyl)cyclohexyl)oxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1,7-dimethyl-1H-indazol-6-yl)piperazin-1-yl)propyl)cyclohexyl)oxy)-2-methylphenyl)picolinate

tert-Butyl 6-(8-(benzo[d]thiazol-2-ylcarbamoyl)-3,4-dihydroisoquinolin-2(1H)-yl)-3-(2-methyl-3-(((1r,4r)-4-(3-oxopropyl)cyclohexyl)oxy)phenyl)picolinate (60 mg, 1 equiv., 82 μmol) and 3-(1,7-dimethyl-6-(piperazin-1-yl)-1H-indazol-3-yl)piperidine-2,6-dione, HCl (34 mg, 1.1 equiv. 90 mol) were suspended in DCM (5 mL). To the mixture was added sodium triacetoxyborohydride (52 mg, 3 equiv., 0.25 mmol). The mixture was stirred at room temperature overnight. The mixture was then concentrated and extracted with DCM and water. The organic layer was separated, washed with brine, dried over Na₂SO₄, filtered, and evaporated to dryness to afford tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1,7-dimethyl-1H-indazol-6-yl)piperazin-1-yl)propyl)cyclohexyl)oxy)-2-methylphenyl)picolinate, which was carried forward without further purification. MS (ESI) m/z: 528.9 [M+2H]²⁺.

### Step B. Procedure for Preparation of 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1,7-dimethyl-1H-indazol-6-yl)piperazin-1-yl)propyl)cyclohexyl)oxy)-2-methylphenyl)picolinic acid

tert-Butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1,7-dimethyl-1H-indazol-6-yl)piperazin-1-yl)propyl)cyclohexyl)oxy)-2-methylphenyl)picolinate (87 mg, 1 equiv., 82 μmol) was dissolved in DCM (4 mL) and TFA (1 mL). The mixture was stirred at room temperature overnight. The reaction mixture was concentrated, and then purified by RP-HPLC to afford 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1,7-dimethyl-1H-indazol-6-yl)piperazin-1-yl)propyl)cyclohexyl)oxy)-2-methylphenyl)picolinic acid (47 mg, 47 μmol, 57%) as a white solid. MS (ESI) m/z: 1000.4 [M+H]⁺; 1H NMR (400 MHz, DMSO-d₆) δ 10.79 (s, 1H), 7.96 (dd, J=7.9, 1.2 Hz, 1H), 7.72 (d, J=8.0 Hz, 1H), 7.56 (dd, J=7.7, 1.4 Hz, 1H), 7.44-7.35 (m, 4H), 7.29 (ddd, J=10.4, 8.8, 7.4 Hz, 2H), 7.01 (t, J=7.9 Hz, 1H), 6.87 (dd, J=15.4, 8.6 Hz, 3H), 6.55 (d, J=7.5 Hz, 1H), 4.91 (s, 2H), 4.26-4.08 (m, 1H), 4.12 (s, 3H), 3.85 (t, J=6.0 Hz, 2H), 2.96 (t, J=6.0 Hz, 2H), 2.82 (s, 4H), 2.57 (s, 3H), 2.66-2.45 (m, 2H), 2.22 (td, J=9.2, 4.6 Hz, 1H), 2.13-1.98 (m, 1H), 2.03 (s, 2H), 1.80 (s, 3H), 1.73 (d, J=12.4 Hz, 2H), 1.30 (d, J=11.2 Hz, 1H), 1.17 (t, J=7.5 Hz, 2H), 1.00 (q, J=12.0 Hz, 2H).

### Example 302. Preparation of Compound 335a

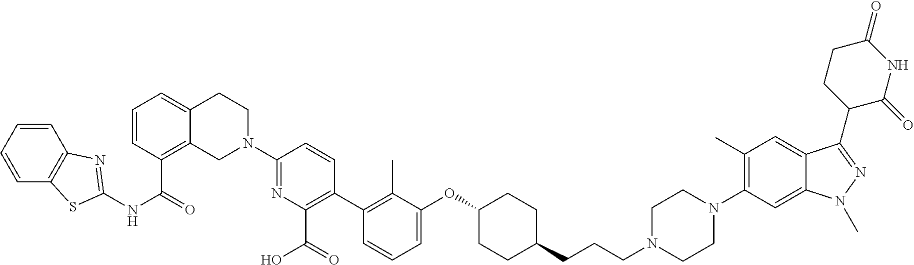 [SMILES: Cc1cc2c(C3CCC(=O)NC3=O)nn(C)c2cc1N1CCN(CCC[C@H]2CC[C@H](Oc3cccc(-c4ccc(N5CCc6cccc(C(=O)Nc7nc8ccccc8s7)c6C5)nc4C(=O)O)c3C)CC2)CC1]

### 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1, 5-dimethyl-1H-indazol-6-yl)piperazin-1-yl)propyl)cyclohexyl)oxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1,5-dimethyl-1H-indazol-6-yl)piperazin-1-yl)propyl)cyclohexyl)oxy)-2-methylphenyl)picolinate

tert-Butyl 6-(8-(benzo[d]thiazol-2-ylcarbamoyl)-3,4-dihydroisoquinolin-2(1H)-yl)-3-(2-methyl-3-(((1r,4r)-4-(3-oxopropyl)cyclohexyl)oxy)phenyl)picolinate (60 mg, 1 equiv. 82 μmol) and 3-(1,5-dimethyl-6-(piperazin-1-yl)-1H-indazol-3-yl)piperidine-2,6-dione, HCl (34 mg, 1.1 equiv. 90 mol) were suspended in DCM (5 mL). To the mixture was added sodium triacetoxyborohydride (52 mg, 3 equiv. 0.25 mmol). The mixture was stirred at room temperature overnight. The mixture was then concentrated and extracted with DCM and water. The organic layer was separated, washed with brine, dried over Na₂SO₄, filtered, and evaporated to dryness to afford tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1, 5-dimethyl-1H-indazol-6-yl)piperazin-1-yl)propyl)cyclohexyl)oxy)-2-methylphenyl)picolinate, which was carried forward without further purification. MS (ESI) m/z: 528.8 [M+2H]²⁺.

### Step B. Procedure for Preparation of 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1,5-dimethyl-1H-indazol-6-yl)piperazin-1-yl)propyl)cyclohexyl)oxy)-2-methylphenyl)picolinic acid

tert-Butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1,5-dimethyl-1H-indazol-6-yl)piperazin-1-yl)propyl)cyclohexyl)oxy)-2-methylphenyl)picolinate (87 mg, 1 equiv. 82 μmol) was dissolved in DCM (4 mL). To the mixture was added TFA (1 mL). The mixture was stirred at room temperature overnight. The reaction mixture was concentrated, and then purified by RP-HPLC to afford 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1, 5-dimethyl-1H-indazol-6-yl)piperazin-1-yl)propyl)cyclohexyl)oxy)-2-methylphenyl)picolinic acid (51 mg, 51 μmol, 62%) as a white solid. MS (ESI) m/z: 1000.6 [M+H]⁺; ¹H NMR (400 MHz, DMSO-d₆) δ 10.77 (s, 1H), 7.95 (d, J=1.3 Hz, OH), 7.72 (d, J=8.0 Hz, 1H), 7.55 (dd, J=7.6, 1.4 Hz, 1H), 7.44-7.34 (m, 4H), 7.34-7.23 (m, 2H), 7.07 (s, 1H), 7.01 (t, J=7.9 Hz, 1H), 6.87 (dd, J=15.0, 8.5 Hz, 2H), 6.55 (d, J=7.5 Hz, 1H), 4.91 (s, 2H), 4.16 (ddd, J=28.0, 10.0, 5.6 Hz, 2H), 3.85 (d, J=3.9 Hz, 5H), 2.96 (t, J=6.0 Hz, 2H), 2.88 (s, 4H), 2.65-2.45 (m, 2H), 2.24 (s, 3H), 2.13-2.02 (m, 1H), 2.03 (s, 1H), 2.00 (s, 1H), 1.80 (s, 3H), 1.74 (d, J=12.4 Hz, 2H), 1.46 (s, 2H), 1.30 (d, J=9.2 Hz, 2H), 1.18 (t, J=7.3 Hz, 3H), 0.99 (dt, J=20.5, 10.1 Hz, 2H).

### Example 303. Preparation of Compound 336a

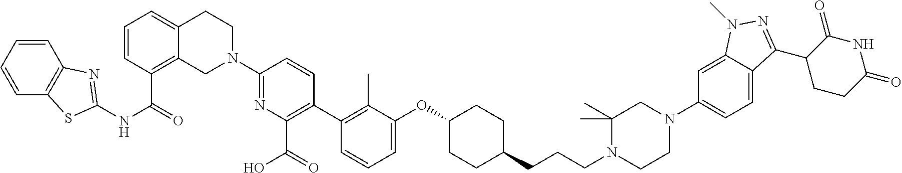 [SMILES: Cc1c(O[C@H]2CC[C@H](CCCN3CCN(c4ccc5c(C6CCC(=O)NC6=O)nn(C)c5c4)CC3(C)C)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)-2,2-dimethylpiperazin-1-yl)propyl)cyclohexyl)oxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)-2,2-dimethylpiperazin-1-yl)propyl)cyclohexyl)oxy)-2-methylphenyl)picolinate

tert-Butyl 6-(8-(benzo[d]thiazol-2-ylcarbamoyl)-3,4-dihydroisoquinolin-2(1H)-yl)-3-(2-methyl-3-(((1r,4r)-4-(3-oxopropyl)cyclohexyl)oxy)phenyl)picolinate (60 mg, 1 equiv. 82 μmol) and 3-(6-(3,3-dimethylpiperazin-1-yl)-1-methyl-1H-indazol-3-yl)piperidine-2,6-dione, HCl (35 mg, 1.1 equiv. 90 μmol) were suspended in DCM (5 mL). To the mixture was added sodium triacetoxyborohydride (52 mg, 3 equiv. 0.25 mmol). The mixture was stirred at room temperature overnight. The mixture was then concentrated and extracted with DCM and water. The organic layer was separated, washed with brine, dried over Na₂SO₄, filtered, and evaporated to dryness to afford tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)-2,2-dimethylpiperazin-1-yl)propyl)cyclohexyl)oxy)-2-methylphenyl)picolinate, which was carried forward without further purification. MS (ESI) m/z: 535.9 [M+2H]²⁺.

### Step B. Procedure for Preparation of 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)-2,2-dimethylpiperazin-1-yl)propyl)cyclohexyl)oxy)-2-methylphenyl)picolinic acid

tert-butyl 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)-2,2-dimethylpiperazin-1-yl)propyl)cyclohexyl)oxy)-2-methylphenyl)picolinate (88 mg, 1 equiv. 82 μmol) was dissolved in DCM (4 mL). To the mixture was added TFA (1 mL). The mixture was stirred at room temperature overnight. The reaction mixture was concentrated, and then purified by RP-HPLC to afford 6-(8-(benzo[d]thiazol-2-ylcarbamoyl)-3,4-dihydroisoquinolin-2(1H)-yl)-3-(3-(((1s,4r)-4-(3-(4-(3-(2,6-dioxopiperidin-3-yl)-1-methyl-1H-indazol-6-yl)-2,2-dimethylpiperazin-1-yl)propyl)cyclohexyl)oxy)-2-methylphenyl)picolinic acid (20 mg, 20 μmol, 24%) as a white solid.

MS (ESI) m/z: 1014.6 [M+H]⁺; ¹H NMR (400 MHz, DMSO-d₆) δ 12.79 (s, 1H), 10.77 (s, 1H), 7.96 (d, J=7.6 Hz, 1H), 7.72 (d, J=8.0 Hz, 1H), 7.55 (d, J=7.6 Hz, 1H), 7.44-7.35 (m, 3H), 7.35-7.24 (m, 2H), 7.01 (t, J=7.9 Hz, 1H), 6.87 (dd, J=15.7, 8.6 Hz, 3H), 6.76 (s, 1H), 6.54 (d, J=7.4 Hz, 1H), 6.44 (s, 1H), 4.91 (s, 2H), 4.19 (dd, J=9.2, 5.2 Hz, 1H), 4.13 (s, 1H), 3.85 (d, J=5.3 Hz, 2H), 3.83 (s, 4H), 2.96 (t, J=6.0 Hz, 2H), 2.88 (s, 2H), 2.56 (d, J=8.5 Hz, 1H), 2.56-2.43 (m, 1H), 2.47 (s, 2H), 2.23 (d, J=9.0 Hz, 1H), 2.09 (dd, J=13.1, 6.0 Hz, 1H), 2.02 (d, J=9.9 Hz, 3H), 1.80 (s, 3H), 1.73 (d, J=12.4 Hz, 2H), 1.34-1.26 (m, 8H), 1.17 (s, 5H), 1.00 (d, J=11.4 Hz, 7H).

### Example 304. Preparation of Compound 337a

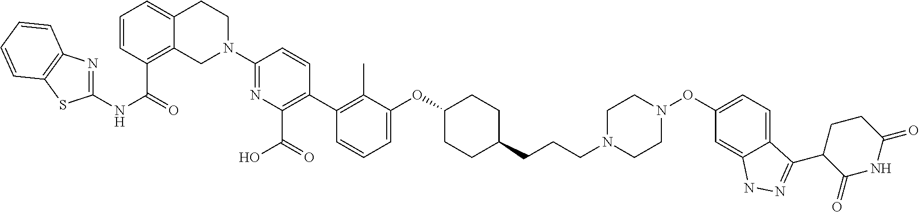 [SMILES: Cc1c(O[C@H]2CC[C@H](CCCN3CCN(Oc4ccc5c(C6CCC(=O)NC6=O)n[nH]c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(3-(4-((3-(2,6-dioxopiperidin-3-yl)-1-methyl-1H-indazol-6-yl)oxy)piperidin-1-yl)propyl)cyclohexyl)oxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(3-(4-((3-(2,6-dioxopiperidin-3-yl)-1-methyl-1H-indazol-6-yl)oxy)piperidin-1-yl)propyl)cyclohexyl)oxy)-2-methylphenyl)picolinate

tert-Butyl 6-(8-(benzo[d]thiazol-2-ylcarbamoyl)-3,4-dihydroisoquinolin-2(1H)-yl)-3-(2-methyl-3-(((1r,4r)-4-(3-oxopropyl)cyclohexyl)oxy)phenyl)picolinate (60 mg, 1 equiv. 82 μmol) and 3-(1-methyl-6-(piperidin-4-yloxy)-1H-indazol-3-yl)piperidine-2,6-dione, HCl (34 mg, 1.1 equiv. 90 mol) were suspended in DCM (5 mL). To the mixture was added sodium triacetoxyborohydride (52 mg, 3 equiv. 0.25 mmol). The mixture was stirred at room temperature overnight. The mixture was then concentrated and extracted with DCM and water. The organic layer was separated, washed with brine, dried over Na₂SO₄, filtered, and evaporated to dryness to afford tert-butyl 6-(8-(benzo[d]thiazol-2-ylcarbamoyl)-3,4-dihydroisoquinolin-2(1H)-yl)-3-(3-(((1s,4r)-4-(3-(4-((3-(2,6-dioxopiperidin-3-yl)-1-methyl-1H-indazol-6-yl)oxy)piperidin-1-yl)propyl)cyclohexyl)oxy)-2-methylphenyl)picolinate, which was carried forward without further purification. MS (ESI) m/z: 529.3 [M+2H]²+.

### Step B. Procedure for Preparation of 6-(8-(benzo[d]thiazol-2-ylcarbamoyl)-3,4-dihydroisoquinolin-2(1H)-yl)-3-(3-(((1s,4r)-4-(3-(4-((3-(2,6-dioxopiperidin-3-yl)-1-methyl-1H-indazol-6-yl)oxy)piperidin-1-yl)propyl)cyclohexyl)oxy)-2-methylphenyl)picolinic acid

tert-Butyl 6-(8-(benzo[d]thiazol-2-ylcarbamoyl)-3,4-dihydroisoquinolin-2(1H)-yl)-3-(3-(((1s,4r)-4-(3-(4-((3-(2,6-dioxopiperidin-3-yl)-1-methyl-1H-indazol-6-yl)oxy)piperidin-1-yl)propyl)cyclohexyl)oxy)-2-methylphenyl)picolinate (87 mg, 1 equiv. 82 μmol) was dissolved in DCM (4 mL). To the mixture was added TFA (1 mL). The mixture was stirred at room temperature overnight. The reaction mixture was concentrated, and then purified by RP-HPLC to afford 6-(8-(benzo[d]thiazol-2-ylcarbamoyl)-3,4-dihydroisoquinolin-2(1H)-yl)-3-(3-(((1s,4r)-4-(3-(4-((3-(2,6-dioxopiperidin-3-yl)-1-methyl-1H-indazol-6-yl)oxy)piperidin-1-yl)propyl)cyclohexyl)oxy)-2-methylphenyl)picolinic acid (40 mg, 40 μmol, 49%) as a white solid. MS (ESI) m/z: 1001.5 [M+H]⁺; 1H NMR (400 MHz, DMSO-d₆) δ 10.79 (s, 1H), 7.96 (dd, J=8.0, 1.2 Hz, 1H), 7.72 (d, J=8.0 Hz, 1H), 7.55 (dd, J=7.6, 1.4 Hz, 1H), 7.50 (d, J=8.8 Hz, 1H), 7.44-7.34 (m, 3H), 7.34-7.23 (m, 2H), 7.06-6.96 (m, 2H), 6.87 (dd, J=14.4, 8.5 Hz, 2H), 6.69 (dd, J=8.7, 2.0 Hz, 1H), 6.55 (d, J=7.5 Hz, 1H), 4.91 (s, 2H), 4.53 (s, 1H), 4.22 (dd, J=9.5, 5.1 Hz, 1H), 4.12 (s, 1H), 3.85 (s, 5H), 3.84 (d, J=5.2 Hz, 1H), 2.95 (t, J=5.9 Hz, 2H), 2.85 (s, 2H), 2.61-2.43 (m, 3H), 2.32-2.18 (m, 1H), 2.15-1.97 (m, 1H), 2.00 (s, 5H), 1.80 (s, 3H), 1.72 (d, J=12.8 Hz, 5H), 1.45 (d, J=9.8 Hz, 3H), 1.30 (d, J=7.7 Hz, OH), 1.14 (t, J=7.8 Hz, 2H), 0.99 (q, J=12.1 Hz, 2H).

### Example 305. Preparation of Compound 338a

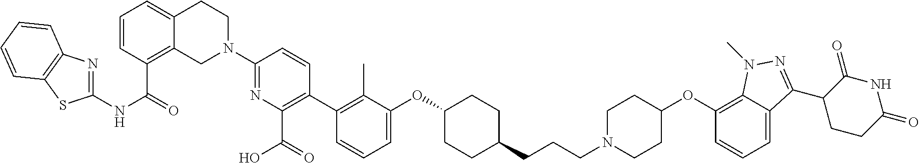 [SMILES: Cc1c(O[C@H]2CC[C@H](CCCN3CCC(Oc4cccc5c(C6CCC(=O)NC6=O)nn(C)c45)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(3-(4-((3-(2,6-dioxopiperidin-3-yl)-1-methyl-1H-indazol-7-yl)oxy)piperidin-1-yl)propyl)cyclohexyl)oxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(3-(4-((3-(2,6-dioxopiperidin-3-yl)-1-methyl-1H-indazol-7-yl)oxy)piperidin-1-yl)propyl)cyclohexyl)oxy)-2-methylphenyl)picolinate

tert-Butyl 6-(8-(benzo[d]thiazol-2-ylcarbamoyl)-3,4-dihydroisoquinolin-2(1H)-yl)-3-(2-methyl-3-(((1r,4r)-4-(3-oxopropyl)cyclohexyl)oxy)phenyl)picolinate (60 mg, 1 equiv. 82 μmol) and 3-(1-methyl-7-(piperidin-4-yloxy)-1H-indazol-3-yl)piperidine-2,6-dione, HCl (34 mg, 1.1 equiv. 90 mol) were suspended in DCM (5 mL). To the mixture was added sodium triacetoxyborohydride (52 mg, 3 equiv. 0.25 mmol). The mixture was stirred at room temperature overnight. The mixture was then concentrated and extracted with DCM and water. The organic layer was separated, washed with brine, dried over Na₂SO₄, filtered, and evaporated to dryness to afford tert-butyl 6-(8-(benzo[d]thiazol-2-ylcarbamoyl)-3,4-dihydroisoquinolin-2(1H)-yl)-3-(3-(((1s,4r)-4-(3-(4-((3-(2,6-dioxopiperidin-3-yl)-1-methyl-1H-indazol-7-yl)oxy)piperidin-1-yl)propyl)cyclohexyl)oxy)-2-methylphenyl)picolinate, which was carried forward without further purification. MS (ESI) m/z: 529.3 [M+2H]²⁺.

### Step B. Procedure for Preparation of 6-(8-(benzo[d]thiazol-2-ylcarbamoyl)-3,4-dihydroisoquinolin-2(1H)-yl)-3-(3-(((1s,4r)-4-(3-(4-((3-(2,6-dioxopiperidin-3-yl)-1-methyl-1H-indazol-7-yl)oxy)piperidin-1-yl)propyl)cyclohexyl)oxy)-2-methylphenyl)picolinic acid

tert-Butyl 6-(8-(benzo[d]thiazol-2-ylcarbamoyl)-3,4-dihydroisoquinolin-2(1H)-yl)-3-(3-(((1s,4r)-4-(3-(4-((3-(2,6-dioxopiperidin-3-yl)-1-methyl-1H-indazol-7-yl)oxy)piperidin-1-yl)propyl)cyclohexyl)oxy)-2-methylphenyl)picolinate (87 mg, 1 equiv. 82 μmol) was dissolved in DCM (4 mL). To the mixture was added TFA (1 mL). The mixture was stirred at room temperature overnight. The reaction mixture was concentrated, and then purified by RP-HPLC to afford 6-(8-(benzo[d]thiazol-2-ylcarbamoyl)-3,4-dihydroisoquinolin-2(1H)-yl)-3-(3-(((1s,4r)-4-(3-(4-((3-(2,6-dioxopiperidin-3-yl)-1-methyl-1H-indazol-7-yl)oxy)piperidin-1-yl)propyl)cyclohexyl)oxy)-2-methylphenyl)picolinic acid (10 mg, 10 μmol, 12%) as a white solid. MS (ESI) m/z: 501.4 [M+2H]²⁺; ¹H NMR (400 MHz, DMSO-d₆) δ 10.80 (s, 1H), 7.96 (d, J=7.5 Hz, 1H), 7.72 (d, J=8.1 Hz, 1H), 7.55 (d, J=7.6 Hz, 1H), 7.44-7.35 (m, 3H), 7.35-7.24 (m, 2H), 7.15 (d, J=8.0 Hz, 1H), 7.00 (t, J=7.9 Hz, 1H), 6.95-6.79 (m, 4H), 6.54 (d, J=7.5 Hz, 1H), 6.44 (s, 1H), 4.91 (s, 2H), 4.25 (dd, J=9.7, 5.1 Hz, 1H), 4.14 (s, 3H), 3.85 (t, J=5.9 Hz, 2H), 3.10 (d, J=5.0 Hz, 2H), 2.96 (s, 2H), 2.60-2.49 (m, 3H), 2.47 (s, 3H), 2.13-2.06 (m, 2H), 2.01 (s, 2H), 1.80 (s, 3H), 1.72 (d, J=12.8 Hz, 3H), 1.29 (s, 1H), 1.16 (s, 2H), 1.00 (d, J=12.1 Hz, 2H).

### Example 306. Preparation of Compound 345a

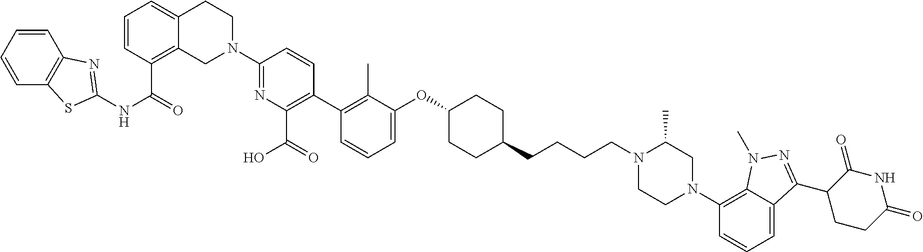 [SMILES: Cc1c(O[C@H]2CC[C@H](CCCCN3CCN(c4cccc5c(C6CCC(=O)NC6=O)nn(C)c45)C[C@H]3C)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(4-((2R)-4-(3-(2,6-dioxopiperidin-3-yl)-1-methyl-1H-indazol-7-yl)-2-methylpiperazin-1-yl)butyl)cyclohexyl)oxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(3-(((1s,4r)-4-(4-((2R)-4-(3-(2,6-dioxopiperidin-3-yl)-1-methyl-1H-indazol-7-yl)-2-methylpiperazin-1-yl)butyl)cyclohexyl)oxy)-2-methylphenyl)picolinate

tert-Butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate (60 mg, 1 equiv. 81 μmol) and 3-(1-methyl-7-((R)-3-methylpiperazin-1-yl)-1H-indazol-3-yl)piperidine-2,6-dione, HCl (37 mg, 1.2 equiv. 97 μmol) were suspended in DCM (4 mL). To the mixture was added sodium triacetoxyborohydride (51 mg, 36 μL, 3 equiv. 0.24 mmol). The mixture was stirred at room temperature overnight. The mixture was then concentrated and extracted with DCM and water. The organic layer was separated, washed with brine, dried over Na₂SO₄, filtered, and evaporated to dryness to afford tert-butyl 6-(8-(benzo[d]thiazol-2-ylcarbamoyl)-3,4-dihydroisoquinolin-2(1H)-yl)-3-(3-(((1s,4r)-4-(4-((2R)-4-(3-(2,6-dioxopiperidin-3-yl)-1-methyl-1H-indazol-7-yl)-2-methylpiperazin-1-yl)butyl)cyclohexyl)oxy)-2-methylphenyl)picolinate, which was carried forward without further purification.

### Step B. Procedure for Preparation of 6-(8-(benzo[d]thiazol-2-ylcarbamoyl)-3,4-dihydroisoquinolin-2(1H)-yl)-3-(3-(((1s,4r)-4-(4-((2R)-4-(3-(2,6-dioxopiperidin-3-yl)-1-methyl-1H-indazol-7-yl)-2-methylpiperazin-1-yl)butyl)cyclohexyl)oxy)-2-methylphenyl)picolinic acid

tert-butyl 6-(8-(benzo[d]thiazol-2-ylcarbamoyl)-3,4-dihydroisoquinolin-2(1H)-yl)-3-(3-(((1s,4r)-4-(4-((2R)-4-(3-(2,6-dioxopiperidin-3-yl)-1-methyl-1H-indazol-7-yl)-2-methylpiperazin-1-yl)butyl)cyclohexyl)oxy)-2-methylphenyl)picolinate (87 mg, 1 equiv. 81 μmol) was dissolved in DCM (4 mL). To the mixture was added TFA (1 mL). The mixture was stirred at room temperature overnight. The reaction mixture was concentrated, and then purified by RP-HPLC to afford 6-(8-(benzo[d]thiazol-2-ylcarbamoyl)-3,4-dihydroisoquinolin-2(1H)-yl)-3-(3-(((1s,4r)-4-(4-((2R)-4-(3-(2,6-dioxopiperidin-3-yl)-1-methyl-1H-indazol-7-yl)-2-methylpiperazin-1-yl)butyl)cyclohexyl)oxy)-2-methylphenyl)picolinic acid (38 mg, 37 μmol, 46%). MS (ESI) m/z: 507.9 [M+2H]²⁺; ¹H NMR (400 MHz, DMSO-d₆) δ 10.81 (s, 1H), 7.96 (d, J=7.8 Hz, 1H), 7.72 (d, J=8.1 Hz, 1H), 7.56 (d, J=7.5 Hz, 1H), 7.40 (t, J=7.6 Hz, 1H), 7.39 (s, 1H), 7.37 (s, 1H), 7.34 (s, 1H), 7.29 (dt, J=10.0, 7.7 Hz, 2H), 7.00 (t, J=7.9 Hz, 1H), 6.96 (d, J=5.1 Hz, 2H), 6.89 (d, J=8.8 Hz, 1H), 6.85 (d, J=8.3 Hz, 1H), 6.54 (d, J=7.5 Hz, 1H), 4.91 (s, 2H), 4.27 (dd, J=9.5, 5.1 Hz, 1H), 4.18 (s, 2H), 4.19-4.10 (m, 1H), 3.85 (t, J=5.9 Hz, 2H), 3.09 (s, 3H), 2.96 (t, J=5.9 Hz, 2H), 2.80 (s, 3H), 2.56 (s, 2H), 2.10 (dd, J=13.4, 5.8 Hz, 1H), 2.01 (t, J=7.2 Hz, 2H), 1.85 (d, J=8.1 Hz, 1H), 1.80 (s, 3H), 1.72 (d, J=12.5 Hz, 2H), 1.43 (s, 3H), 1.28 (s, 4H), 1.19 (s, 4H), 1.06-0.95 (m, 5H).

### Example 307. Preparation of Compound 351a

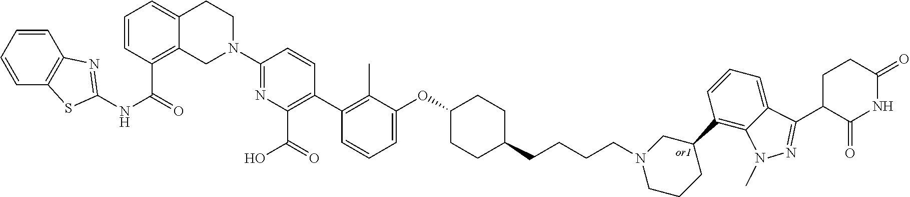 [SMILES: Cc1c(O[C@H]2CC[C@H](CCCCN3CCC[C@H](c4cccc5c(C6CCC(=O)NC6=O)nn(C)c45)C3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1 S,4r)-4-(4-((3R)-3-(3-(2,6-di oxopiperidin-3-yl)-1-methyl-1H-indazol-7-yl)piperidin-1l-yl)butyl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1S,4r)-4-(4-((3R*)-3-(3-(2, 6-dioxopiperidin-3-yl)-1-methyl-1H-indazol-7-yl)piperidin-1l-yl)butyl)cyclohexyl)oxy)-2-methylphenyl)picolinic acid]

### Step A. Procedure for Preparation of tert-butyl 3-(3-(2,6-bis(benzyloxy)pyridin-3-yl)-1-methyl-1H-indazol-7-yl)-4-oxopiperidine-1-carboxylate

A mixture of 3-(2,6-bis(benzyloxy)pyridin-3-yl)-7-bromo-1-methyl-1H-indazole (100 g, 199 mmol, 1.00 equiv.), compound tert-butyl 4-oxopiperidine-1-carboxylate (119 g, 599 mmol, 3.00 equiv.), ^{t}BuONa (57.6 g, 599 mmol, 3.00 equiv.), RuPhos (27.9 g, 59.9 mmol, 0.30 equiv.) in 1,4-dioxane (750 mL) was degassed and purged with N₂ three times. To this solution was added Pd₂(dba)₃ (27.4 g, 29.9 mmol, 0.15 equiv.), and the mixture was stirred at 65° C. for 2 hours under N₂ atmosphere. The reaction mixture was poured into water (1.00 L) and extracted with ethyl acetate (1.00 L×3). The combined organic phases were concentrated in vacuo to give a residue. The residue was purified by column chromatography (petroleum ether: ethyl acetate=8:1 to 1:1) to give tert-butyl 3-(3-(2,6-bis(benzyloxy)pyridin-3-yl)-1-methyl-1H-indazol-7-yl)-4-oxopiperidine-1-carboxylate (200 g, 323 mmol, 80.7% yield) as a yellow solid.

MS (ESI) m/z=619.4 [M+H]⁺

### Step B. Procedure for Preparation of tert-butyl 3-(3-(2,6-bis(benzyloxy)pyridin-3-yl)-1-methyl-1H-indazol-7-yl)piperidine-1-carboxylate

To a solution of tert-butyl 3-(3-(2,6-bis(benzyloxy)pyridin-3-yl)-1-methyl-1H-indazol-7-yl)-4-oxopiperidine-1-carboxylate (55.0 g, 88.9 mmol, 1.00 equiv.) in diglycol (550 mL) was added N₂H₄-H₂O (41.8 g, 711 mmol, 40.6 mL, 85.0% purity, 8.00 equiv.) and KOH (29.9 g, 533 mmol, 6.00 equiv.). The mixture was stirred at 120° C. for 6 hrs. The reaction mixture was quenched by addition of 0.50 M HCl (1.20 L) and then extracted with ethyl acetate (1.60 L×3). The combined organic layers were washed with brine (1.20 L×2), dried over Na₂SO₄, filtered, and concentrated under reduced pressure to give a residue. The residue was purified by column chromatography (petroleum ether:ethyl acetate=1:0 to 2:1) to give tert-butyl 3-(3-(2,6-bis(benzyloxy)pyridin-3-yl)-1-methyl-1H-indazol-7-yl)piperidine-1-carboxylate (48.0 g, 78.6 mmol, 44.3% yield) as a yellow solid. MS (ESI) m/z=605.6 (M+H)⁺

### Step C. Procedure for Preparation of tert-butyl (R)-3-(3-(2,6-bis(benzyloxy)pyridin-3-yl)-1-methyl-1H-indazol-7-yl)piperidine-1-carboxylate and tert-butyl (S)-3-(3-(2,6-bis(benzyloxy)pyridin-3-yl)-1-methyl-1H-indazol-7-yl)piperidine-1-carboxylate

Stereoisomers of tert-butyl 3-(3-(2,6-bis(benzyloxy)pyridin-3-yl)-1-methyl-1H-indazol-7-yl)piperidine-1-carboxylate (56.5 g, 93.4 mmol) were separated by SFC to give tert-butyl (R)-3-(3-(2,6-bis(benzyloxy)pyridin-3-yl)-1-methyl-1H-indazol-7-yl)piperidine-1-carboxylate (27.4 g, 45.31 mmol, 48.5% yield) and tert-butyl (S)-3-(3-(2,6-bis(benzyloxy)pyridin-3-yl)-1-methyl-1H-indazol-7-yl)piperidine-1-carboxylate (26.3 g, 43.4 mmol, 46.5% yield) as yellow solids. MS (ESI) m/z=605.8 [M+H]⁺. MS (ESI) m/z=605.8 [M+H]⁺

### Step D. Procedure for Preparation of tert-butyl (3R)-3-(3-(2,6-dioxopiperidin-3-yl)-1-methyl-1H-indazol-7-yl)piperidine-1-carboxylate

To a solution of tert-butyl (R)-3-(3-(2,6-bis(benzyloxy)pyridin-3-yl)-1-methyl-1H-indazol-7-yl)piperidine-1-carboxylate (13.0 g, 21.5 mmol, 1.00 equiv.), and Pd/C (6.50 g, 6.11 mmol, 10.0% purity), Pd(OH)₂ (6.50 g, 9.26 mmol, 20.0% purity) in EtOH (130 mL), THE (130 mL) under Ar. The suspension was degassed under vacuum and purged with H₂ three times. The mixture was stirred under H₂ (50 psi) at 50° C. for 2 hrs. The reaction mixture was filtered and washed with ethyl acetate (300 mL), and concentrated under reduced pressure to give tert-butyl (3R)-3-(3-(2,6-dioxopiperidin-3-yl)-1-methyl-1H-indazol-7-yl)piperidine-1-carboxylate (9.00 g, 9.25 mmol, 100% yield) as a light yellow solid. MS (ESI) m/z=427.2 [M+H]⁺

### Step E. Procedure for Preparation of 3-(1-methyl-7-((R)-piperidin-3-yl)-1H-indazol-3-yl)piperidine-2,6-dione

A mixture of tert-butyl (3R)-3-(3-(2,6-dioxopiperidin-3-yl)-1-methyl-1H-indazol-7-yl)piperidine-1-carboxylate (8.00 g, 18.7 mmol, 1.00 equiv.) in HCl/dioxane (80.0 mL) was stirred at 25° C. for 1 hr. The reaction mixture was filtered and concentrated under reduced pressure to give 3-(1-methyl-7-((R)-piperidin-3-yl)-1H-indazol-3-yl)piperidine-2,6-dione (8.49 g, 25.6 mmol, 51.6% yield) as a white solid. MS (ESI) m/z=327.3 [M+H]⁺. ¹H NMR: (400 MHz, DMSO-d₆) δ 10.89 (s, 1H), 9.78 (br d, J=10.1 Hz, 1H), 9.43 (br s, 1H), 7.60 (d, J=8.0 Hz, 1H), 7.33 (d, J=7.1 Hz, 1H), 7.10 (t, J=7.6 Hz, 1H), 4.37 (dd, J=4.9, 9.9 Hz, 1H), 4.27 (s, 3H), 4.05 (br t, J=11.8 Hz, 1H), 3.41-3.28 (m, 2H), 3.16-3.06 (m, 1H), 3.02-2.87 (m, 1H), 2.74-2.55 (m, 2H), 2.41-2.27 (m, 1H), 2.15 (br dd, J=5.5, 13.1 Hz, 1H), 2.06-1.96 (m, 2H), 1.88 (br d, J=14.5 Hz, 2H)

### Step F. Procedure for Preparation of tert-butyl 6-(8-(benzo[d]thiazol-2-ylcarbamoyl)-3,4-dihydroisoquinolin-2(1H)-yl)-3-(3-(((1S,4r)-4-(4-((3R)-3-(3-(2,6-dioxopiperidin-3-yl)-1-methyl-1H-indazol-7-yl)piperidin-1-yl)butyl)cyclohexyl)oxy)-2-methylphenyl)picolinate

tert-Butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate (60 mg, 1 equiv. 81 μmol) and 3-(1-methyl-7-((R)-piperidin-3-yl)-1H-indazol-3-yl)piperidine-2,6-dione, HCl (35 mg, 1.2 equiv. 97 mol) were suspended in DCM (5 mL). To the mixture was added sodium triacetoxyborohydride (51 mg, 36 μL, 3 equiv. 0.24 mmol). The mixture was stirred at room temperature overnight. The mixture was then concentrated and extracted with DCM and water. The organic layer was separated, washed with brine, dried over Na₂SO₄, filtered, and evaporated to dryness to afford tert-butyl 6-(8-(benzo[d]thiazol-2-ylcarbamoyl)-3,4-dihydroisoquinolin-2(1H)-yl)-3-(3-(((1S,4r)-4-(4-((3R)-3-(3-(2,6-dioxopiperidin-3-yl)-1-methyl-1H-indazol-7-yl)piperidin-1-yl)butyl)cyclohexyl)oxy)-2-methylphenyl)picolinate, which was carried forward without further purification. MS (ESI) m/z: 528.5 [M+2H]²⁺.

### Step G. Procedure for Preparation of 6-(8-(benzo[d]thiazol-2-ylcarbamoyl)-3,4-dihydroisoquinolin-2(1H)-yl)-3-(3-(((1S,4r)-4-(4-((3R)-3-(3-(2,6-dioxopiperidin-3-yl)-1-methyl-1H-indazol-7-yl)piperidin-1-yl)butyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1S,4r)-4-(4-((3R*)-3-(3-(2,6-dioxopiperidin-3-yl)-1-methyl-1H-indazol-7-yl)piperidin-1-yl)butyl)cyclohexyl)oxy)-2-methylphenyl)picolinic acid]

tert-Butyl 6-(8-(benzo[d]thiazol-2-ylcarbamoyl)-3,4-dihydroisoquinolin-2(1H)-yl)-3-(3-(((1S,4r)-4-(4-((3R)-3-(3-(2,6-dioxopiperidin-3-yl)-1-methyl-1H-indazol-7-yl)piperidin-1-yl)butyl)cyclohexyl)oxy)-2-methylphenyl)picolinatetert (85 mg, 1 equiv. 81 μmol) was dissolved in DCM (4 mL). To the mixture was added 2 mL TFA. The mixture was stirred at room temperature overnight. The reaction mixture was concentrated, and then purified by RP-HPLC to afford 6-(8-(benzo[d]thiazol-2-ylcarbamoyl)-3,4-dihydroisoquinolin-2(1H)-yl)-3-(3-(((1S,4r)-4-(4-((3R)-3-(3-(2,6-dioxopiperidin-3-yl)-1-methyl-1H-indazol-7-yl)piperidin-1-yl)butyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1S,4r)-4-(4-((3R*)-3-(3-(2,6-dioxopiperidin-3-yl)-1-methyl-1H-indazol-7-yl)piperidin-1-yl)butyl)cyclohexyl)oxy)-2-methylphenyl)picolinic acid] (38 mg, 38 μmol, 47%) as a white solid. MS (ESI) m/z: 500.4 [M+2H]²⁺; ¹H NMR (400 MHz, DMSO-d₆) δ 10.81 (s, 1H), 7.95 (d, J=7.9 Hz, 1H), 7.71 (d, J=8.1 Hz, 1H), 7.53 (dd, J=18.8, 7.8 Hz, 2H), 7.44-7.34 (m, 3H), 7.29 (dt, J=11.1, 7.5 Hz, 2H), 7.22 (d, J=7.1 Hz, 1H), 7.00 (q, J=7.8 Hz, 2H), 6.88 (d, J=8.8 Hz, 1H), 6.83 (d, J=8.3 Hz, 1H), 6.55 (d, J=7.4 Hz, 1H), 4.91 (s, 2H), 4.28 (dd, J=9.9, 5.1 Hz, 1H), 4.14 (s, 3H), 4.11 (s, 1H), 3.84 (t, J=6.0 Hz, 2H), 3.59 (s, 1H), 3.04 (s, 1H), 2.95 (t, J=5.9 Hz, 2H), 2.61-2.45 (m, 2H), 2.33-2.20 (m, 1H), 2.08 (dd, J=13.5, 5.5 Hz, 1H), 1.99 (d, J=10.0 Hz, 2H), 1.92-1.77 (m, 4H), 1.77-1.65 (m, 4H), 1.60 (s, 1H), 1.43 (s, 2H), 1.22 (d, J=6.7 Hz, OH), 1.14 (d, J=6.4 Hz, 1H), 0.98 (t, J=12.2 Hz, 2H).

### Example 308. Preparation of Compound 351b

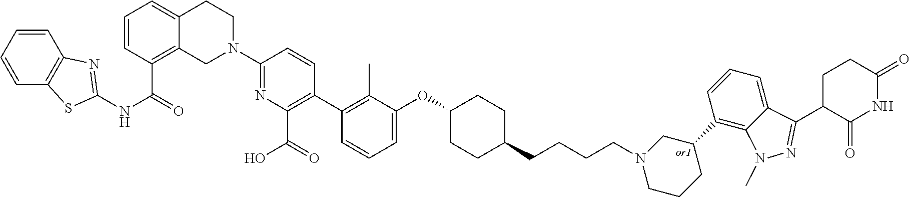 [SMILES: Cc1c(O[C@H]2CC[C@H](CCCCN3CCC[C@@H](c4cccc5c(C6CCC(=O)NC6=O)nn(C)c45)C3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1R,4r)-4-(4-((3S)-3-(3-(2,6-dioxopiperidin-3-yl)-1-methyl-1H-indazol-7-yl)piperidin-1-yl)butyl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1R,4r)-4-(4-((3S*)-3-(3-(2,6-dioxopiperidin-3-yl)-1-methyl-1H-indazol-7-yl)piperidin-1-yl)butyl)cyclohexyl)oxy)-2-methylphenyl)picolinic acid]

### Step A. Procedure for Preparation of tert-butyl (3S)-3-(3-(2,6-dioxopiperidin-3-yl)-1-methyl-1H-indazol-7-yl)piperidine-1-carboxylate

A solution of tert-butyl (S)-3-(3-(2,6-bis(benzyloxy)pyridin-3-yl)-1-methyl-1H-indazol-7-yl)piperidine-1-carboxylate (23.0 g, 38.0 mmol, 1.00 equiv.) and Pd/C (11.5 g, 12.2 mmol, 10.0% purity), Pd(OH)₂ (11.5 g, 18.5 mmol, 20.0% purity) in EtOH (230 mL), THE (230 mL) under Ar. was degassed under vacuum and purged with H₂ three times. The mixture was stirred under H₂ (50 psi) at 50° C. for 2 hrs. The reaction mixture was filtered and washed with ethyl acetate (300 mL), then concentrated under reduced pressure to give tert-butyl (3S)-3-(3-(2,6-dioxopiperidin-3-yl)-1-methyl-1H-indazol-7-yl)piperidine-1-carboxylate (18.0 g, 48.4 mmol, 100% yield) as a light-yellow solid. MS (ESI) m/z=427.2 [M+H]⁺

### Step B. Procedure for Preparation of 3-(1-methyl-7-((S)-piperidin-3-yl)-1H-indazol-3-yl)piperidine-2,6-dione

A mixture of tert-butyl (3S)-3-(3-(2,6-dioxopiperidin-3-yl)-1-methyl-1H-indazol-7-yl)piperidine-1-carboxylate (15.0 g, 35.1 mmol, 1.00 eq)) in HCl/dioxane (150 mL) was stirred at 25° C. for 1 hr. The reaction mixture was filtered and concentrated under reduced pressure to give 3-(1-methyl-7-((S)-piperidin-3-yl)-1H-indazol-3-yl)piperidine-2,6-dione (8.49 g, 25.9 mmol, 61.9% yield, HCl) as light yellow solid. MS (ESI) m/z=327.3 [M+H]⁺. ¹H NMR: (400 MHz, DMSO-d₆) δ 10.90 (s, 1H), 9.76 (br d, J=10.4 Hz, 1H), 9.41 (br s, 1H), 7.61 (d, J=8.0 Hz, 1H), 7.33 (d, J=7.3 Hz, 1H), 7.10 (t, J=7.6 Hz, 1H), 4.37 (dd, J=4.9, 9.9 Hz, 1H), 4.27 (s, 3H), 4.11-4.00 (m, 1H), 3.42-3.28 (m, 2H), 3.14 (q, J=11.6 Hz, 1H), 3.01-2.88 (m, 1H), 2.74-2.55 (m, 2H), 2.34 (ttd, J=4.7, 9.3, 13.7 Hz, 1H), 2.20-2.10 (m, 1H), 2.07-1.96 (m, 2H), 1.94-1.80 (m, 2H).

### Step C. Procedure for Preparation of tert-butyl 6-(8-(benzo[d]thiazol-2-ylcarbamoyl)-3,4-dihydroisoquinolin-2(1H)-yl)-3-(3-(((1R,4r)-4-(4-((3S)-3-(3-(2,6-dioxopiperidin-3-yl)-1-methyl-1H-indazol-7-yl)piperidin-1-yl)butyl)cyclohexyl)oxy)-2-methylphenyl)picolinate

tert-Butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate (60 mg, 1 equiv. 81 μmol) and rel-3-(1-methyl-7-((R)-piperidin-3-yl)-1H-indazol-3-yl)piperidine-2,6-dione, HCl (35 mg, 1.2 equiv. 97 μmol) were suspended in DCM (5 mL). To the mixture was added sodium triacetoxyborohydride (51 mg, 36 μL, 3 equiv. 0.24 mmol). The mixture was stirred at room temperature overnight. The mixture was then concentrated and extracted with DCM and water. The organic layer was separated, washed with brine, dried over Na₂SO₄, filtered, and evaporated to dryness to afford tert-butyl 6-(8-(benzo[d]thiazol-2-ylcarbamoyl)-3,4-dihydroisoquinolin-2(1H)-yl)-3-(3-(((1R,4r)-4-(4-((3S)-3-(3-(2,6-dioxopiperidin-3-yl)-1-methyl-1H-indazol-7-yl)piperidin-1-yl)butyl)cyclohexyl)oxy)-2-methylphenyl)picolinate, which was carried forward without further purification. MS (ESI) m/z: 528.5 [M+2H]²⁺.

### Step D. Procedure for Preparation of 6-(8-(benzo[d]thiazol-2-ylcarbamoyl)-3,4-dihydroisoquinolin-2(1H)-yl)-3-(3-(((1R,4r)-4-(4-((3S)-3-(3-(2,6-dioxopiperidin-3-yl)-1-methyl-1H-indazol-7-yl)piperidin-1-yl)butyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1R,4r)-4-(4-((3S*)-3-(3-(2,6-dioxopiperidin-3-yl)-1-methyl-1H-indazol-7-yl)piperidin-1-yl)butyl)cyclohexyl)oxy)-2-methylphenyl)picolinic acid]

tert-Butyl 6-(8-(benzo[d]thiazol-2-ylcarbamoyl)-3,4-dihydroisoquinolin-2(1H)-yl)-3-(3-(((1R,4r)-4-(4-((3S)-3-(3-(2,6-dioxopiperidin-3-yl)-1-methyl-1H-indazol-7-yl)piperidin-1-yl)butyl)cyclohexyl)oxy)-2-methylphenyl)picolinate (85 mg, 1 equiv. 81 μmol) was dissolved in DCM (4 mL). To the mixture was added 2 mL TFA. The mixture was stirred at room temperature overnight. The reaction mixture was concentrated, and then purified by RP-HPLC to afford 6-(8-(benzo[d]thiazol-2-ylcarbamoyl)-3,4-dihydroisoquinolin-2(1H)-yl)-3-(3-(((1R,4r)-4-(4-((3S)-3-(3-(2,6-dioxopiperidin-3-yl)-1-methyl-1H-indazol-7-yl)piperidin-1-yl)butyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1R,4r)-4-(4-((3S*)-3-(3-(2,6-dioxopiperidin-3-yl)-1-methyl-1H-indazol-7-yl)piperidin-1-yl)butyl)cyclohexyl)oxy)-2-methylphenyl)picolinic acid] (42 mg, 42 μmol, 52%) as a white solid. MS (ESI) m/z: 500.4 [M+2H]²⁺; ¹H NMR (400 MHz, DMSO-d₆) δ 10.81 (s, 1H), 7.95 (d, J=7.9 Hz, 1H), 7.71 (d, J=8.1 Hz, 1H), 7.53 (dd, J=19.7, 7.8 Hz, 2H), 7.44-7.17 (m, 7H), 7.00 (q, J=7.8 Hz, 2H), 6.87 (d, J=8.8 Hz, 1H), 6.86-6.76 (m, 1H), 6.55 (d, J=7.5 Hz, 1H), 4.91 (s, 2H), 4.28 (dt, J=8.4, 4.2 Hz, 1H), 4.13 (d, J=7.6 Hz, 4H), 4.01 (s, 1H), 3.84 (t, J=6.0 Hz, 2H), 3.59 (s, 1H), 3.10 (s, 2H), 3.03 (s, 1H), 2.95 (t, J=6.0 Hz, 2H), 2.61-2.45 (m, 2H), 2.34-2.20 (m, 1H), 2.13-2.04 (m, 1H), 2.04-1.96 (m, 2H), 1.82 (d, J=17.4 Hz, 4H), 1.69 (d, J=13.3 Hz, 3H), 1.42 (s, 2H), 1.24 (dd, J=21.7, 9.4 Hz, 1H), 1.17-1.09 (m, 1H), 0.98 (t, J=12.3 Hz, 2H).

### Example 309. Preparation of Compound 352a

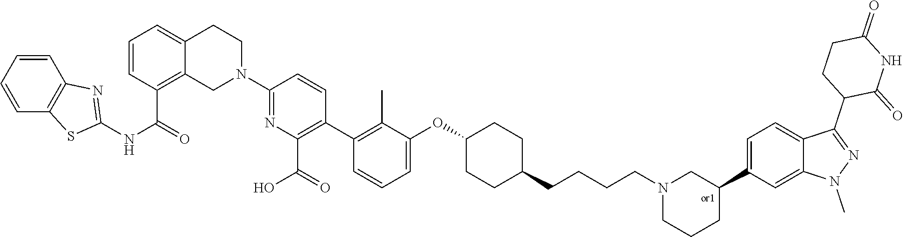 [SMILES: Cc1c(O[C@H]2CC[C@H](CCCCN3CCC[C@H](c4ccc5c(C6CCC(=O)NC6=O)nn(C)c5c4)C3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1S,4r)-4-(4-((3R)-3-(3-(2,6-dioxopiperidin-3-yl)-1-methyl-1H-indazol-6-yl)piperidin-1-yl)butyl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1S,4r)-4-(4-((3R*)-3-(3-(2,6-dioxopiperidin-3-yl)-1-methyl-1H-indazol-6-yl)piperidin-1-yl)butyl)cyclohexyl)oxy)-2-methylphenyl)picolinic acid]

### Step A. Procedure for Preparation of tert-butyl 3-(3-(2,6-bis(benzyloxy)pyridin-3-yl)-1-methyl-1H-indazol-6-yl)-4-oxopiperidine-1-carboxylate

A mixture of 3-(2,6-bis(benzyloxy)pyridin-3-yl)-6-bromo-1-methyl-1H-indazole (50.0 g, 99.9 mmol, 1.00 equiv.), compound tert-butyl 4-oxopiperidine-1-carboxylate (59.7 g, 299 mmol, 3.00 equiv.), ^{t}BuONa (28.8 g, 299 mmol, 3.00 equiv.), and RuPhos (13.9 g, 29.9 mmol, 0.30 equiv.) in 1,4-dioxane (500 mL) was degassed and purged with N₂ three times. Then, Pd₂(dba)₃ (13.7 g, 14.9 mmol, 0.15 equiv.) was added, and the mixture was stirred at 65° C. for 2 hours under N₂ atmosphere. The reaction mixture was poured into water (1.00 L) and extracted with ethyl acetate (1.00 L×3). The combined organic phase was concentrated in vacuo to give a residue. The residue was purified by column chromatography (petroleum ether: ethyl acetate=8:1 to 1:1) to give tert-butyl 3-(3-(2,6-bis(benzyloxy)pyridin-3-yl)-1-methyl-1H-indazol-6-yl)-4-oxopiperidine-1-carboxylate (100 g, 146 mmol, 48.9% yield) as a yellow solid. MS (ESI) m/z=619.6 [M+H]⁺.

MS (ESI) m/z=619.6 [M+H]⁺

### Step B. Procedure for Preparation of tert-butyl 3-(3-(2,6-bis(benzyloxy)pyridin-3-yl)-1-methyl-1H-indazol-6-yl)piperidine-1-carboxylate

To a solution of tert-butyl 3-(3-(2,6-bis(benzyloxy)pyridin-3-yl)-1-methyl-1H-indazol-6-yl)-4-oxopiperidine-1-carboxylate (35.0 g, 56.5 mmol, 1.00 equiv.) in diglycol (350 mL) was added N₂H₄H₂O (26.6 g, 452 mmol, 25.8 mL, 85.0% purity, 8.00 equiv.) and KOH (19.0 g, 339 mmol, 6.00 equiv.). The mixture was stirred at 120° C. for 6 hrs. The reaction mixture was quenched by addition of 0.50 M HCl (500 mL), and then extracted with ethyl acetate (500 mL×3). The combined organic layers were washed with brine (250 mL×2), dried over Na₂SO₄, filtered, and concentrated under reduced pressure to give a residue. The residue was purified by column chromatography (petroleum ether: ethyl acetate=5:1 to 1:1) to give tert-butyl 3-(3-(2,6-bis(benzyloxy)pyridin-3-yl)-1-methyl-1H-indazol-6-yl)piperidine-1-carboxylate (31.0 g, 51.1 mmol, 37.2% yield) as a yellow solid. MS (ESI) m/z=605.7 [M+H]⁺

### Step C. Procedure for Preparation of tert-butyl (R)-3-(3-(2,6-bis(benzyloxy)pyridin-3-yl)-1-methyl-1H-indazol-6-yl)piperidine-1-carboxylate and tert-butyl (S)-3-(3-(2,6-bis(benzyloxy)pyridin-3-yl)-1-methyl-1H-indazol-6-yl)piperidine-1-carboxylate

The stereosomers of tert-butyl 3-(3-(2,6-bis(benzyloxy)pyridin-3-yl)-1-methyl-1H-indazol-6-yl)piperidine-1-carboxylate (31.0 g, 51.1 mmol) were separated by SFC to give tert-butyl (R)-3-(3-(2,6-bis(benzyloxy)pyridin-3-yl)-1-methyl-1H-indazol-6-yl)piperidine-1-carboxylate (13.0 g, 21.50 mmol, 41.9% yield) and tert-butyl (S)-3-(3-(2,6-bis(benzyloxy)pyridin-3-yl)-1-methyl-1H-indazol-6-yl)piperidine-1-carboxylate (13.0 g, 21.50 mmol, 41.9% yield) as light-yellow solids.

### Step D. Procedure for Preparation of tert-butyl (3R)-3-(3-(2,6-dioxopiperidin-3-yl)-1-methyl-1H-indazol-6-yl)piperidine-1-carboxylate

A solution of tert-butyl (R)-3-(3-(2,6-bis(benzyloxy)pyridin-3-yl)-1-methyl-1H-indazol-6-yl)piperidine-1-carboxylate (13.0 g, 21.5 mmol, 1.00 equiv.), and Pd/C (6.50 g, 6.11 mmol, 10.0% purity), Pd(OH)₂ (6.50 g, 9.26 mmol, 20.0% purity) in EtOH (130 mL), THE (130 mL) under Ar was degassed under vacuum and purged with H₂ three times. The mixture was stirred under H₂ (50 psi) at 50° C. for 2 hrs. The reaction mixture was filtered, washed with ethyl acetate (300 mL), and concentrated under reduced pressure to give compound tert-butyl (3R)-3-(3-(2,6-dioxopiperidin-3-yl)-1-methyl-1H-indazol-6-yl)piperidine-1-carboxylate (6.20 g, 14.4 mmol, 67.2% yield, 99.4% purity) as a white solid. MS (ESI) m/z=427.3 [M+H]⁺

### Step E. Procedure for Preparation of 3-(1-methyl-6-((R)-piperidin-3-yl)-1H-indazol-3-yl)piperidine-2,6-dione

A mixture of compound tert-butyl (3R)-3-(3-(2,6-dioxopiperidin-3-yl)-1-methyl-1H-indazol-6-yl)piperidine-1-carboxylate (5.00 g, 11.7 mmol, 1.00 eq) in HCl/dioxane (50.0 mL) was stirred at 25° C. for 1 hour. The reaction mixture was filtered and concentrated under reduced pressure to give 3-(1-methyl-6-((R)-piperidin-3-yl)-1H-indazol-3-yl)piperidine-2,6-dione (4.62 g, 14.1 mmol, 100% yield, 98.0% purity) as a white solid. MS (ESI) m/z=327.3 [M+H]⁺. ¹H NMR: (400 MHz, DMSO-d₆) δ 11.02-10.77 (m, 1H), 9.68-9.32 (m, 2H), 7.67 (d, J=8.4 Hz, 1H), 7.52 (s, 1H), 7.06 (br d, J=8.4 Hz, 1H), 4.41-4.31 (m, 1H), 3.98 (s, 3H), 3.35-3.05 (m, 4H), 2.98-2.81 (m, 1H), 2.75-2.53 (m, 2H), 2.42-2.27 (m, 1H), 2.23-2.08 (m, 1H), 1.98-1.70 (m, 4H)

### Step F. Procedure for Preparation of tert-butyl 6-(8-(benzo[d]thiazol-2-ylcarbamoyl)-3,4-dihydroisoquinolin-2(1H)-yl)-3-(3-(((1S,4r)-4-(4-((3R)-3-(3-(2,6-dioxopiperidin-3-yl)-1-methyl-1H-indazol-6-yl)piperidin-1-yl)butyl)cyclohexyl)oxy)-2-methylphenyl)picolinate

tert-Butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate (60 mg, 1 equiv. 81 μmol) and 3-(1-methyl-6-((R)-piperidin-3-yl)-1H-indazol-3-yl)piperidine-2,6-dione, HCl (35 mg, 1.2 equiv. 97 mol) were suspended in DCM (5 mL). To the mixture was added sodium triacetoxyborohydride (51 mg, 36 μL, 3 equiv. 0.24 mmol). The mixture was stirred at room temperature overnight. The mixture was then concentrated and extracted with DCM and water. The organic layer was separated, washed with brine, dried over Na₂SO₄, filtered, and evaporated to dryness to afford tert-butyl 6-(8-(benzo[d]thiazol-2-ylcarbamoyl)-3,4-dihydroisoquinolin-2(1H)-yl)-3-(3-(((1S,4r)-4-(4-((3R)-3-(3-(2,6-dioxopiperidin-3-yl)-1-methyl-1H-indazol-6-yl)piperidin-1-yl)butyl)cyclohexyl)oxy)-2-methylphenyl)picolinate, which was carried forward without further purification. MS (ESI) m/z: 528.4 [M+2H]²⁺.

### Step G. Procedure for Preparation of 6-(8-(benzo[d]thiazol-2-ylcarbamoyl)-3,4-dihydroisoquinolin-2(1H)-yl)-3-(3-(((1S,4r)-4-(4-((3R)-3-(3-(2,6-dioxopiperidin-3-yl)-1-methyl-1H-indazol-6-yl)piperidin-1-yl)butyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1S,4r)-4-(4-((3R*)-3-(3-(2,6-dioxopiperidin-3-yl)-1-methyl-1H-indazol-6-yl)piperidin-1-yl)butyl)cyclohexyl)oxy)-2-methylphenyl)picolinic acid]

tert-Butyl 6-(8-(benzo[d]thiazol-2-ylcarbamoyl)-3,4-dihydroisoquinolin-2(1H)-yl)-3-(3-(((1S,4r)-4-(4-((3R)-3-(3-(2,6-dioxopiperidin-3-yl)-1-methyl-1H-indazol-6-yl)piperidin-1-yl)butyl)cyclohexyl)oxy)-2-methylphenyl)picolinate (85 mg, 1 equiv. 81 μmol) was dissolved in DCM (4 mL). To the mixture was added 2 mL TFA. The mixture was stirred at room temperature overnight. The reaction mixture was concentrated, and then purified by RP-HPLC to afford 6-(8-(benzo[d]thiazol-2-ylcarbamoyl)-3,4-dihydroisoquinolin-2(1H)-yl)-3-(3-(((1S,4r)-4-(4-((3R)-3-(3-(2,6-dioxopiperidin-3-yl)-1-methyl-1H-indazol-6-yl)piperidin-1-yl)butyl)cyclohexyl)oxy)-2-methylphenyl)picolinic acid [6-(8-(benzo[d]thiazol-2-ylcarbamoyl)-3,4-dihydroisoquinolin-2(1H)-yl)-3-(3-(((1S,4r)-4-(4-((3R*)-3-(3-(2,6-dioxopiperidin-3-yl)-1-methyl-1H-indazol-6-yl)piperidin-1-yl)butyl)cyclohexyl)oxy)-2-methylphenyl)picolinic acid] (43 mg, 43 μmol, 53%) as a white solid. MS (ESI) m/z: 500.4 [M+2H]²⁺; ¹H NMR (400 MHz, DMSO-d₆) δ 10.81 (s, 1H), 7.95 (d, J=7.9 Hz, 1H), 7.72 (d, J=8.1 Hz, 1H), 7.56 (dd, J=8.2, 3.1 Hz, 2H), 7.38 (dt, J=11.3, 3.8 Hz, 4H), 7.29 (dt, J=10.7, 7.6 Hz, 2H), 7.03-6.93 (m, 2H), 6.87 (d, J=8.8 Hz, 1H), 6.83 (d, J=8.3 Hz, 1H), 6.55 (d, J=7.4 Hz, 1H), 4.91 (s, 2H), 4.27 (dd, J=9.8, 5.0 Hz, 1H), 4.10 (tt, J=10.5, 4.4 Hz, 1H), 3.90 (s, 3H), 3.84 (t, J=6.0 Hz, 2H), 3.05 (s, 2H), 2.95 (t, J=6.0 Hz, 2H), 2.90 (s, 1H), 2.67-2.45 (m, 2H), 2.34-2.21 (m, 1H), 2.09 (dq, J=12.9, 5.2 Hz, 1H), 2.03-1.95 (m, 2H), 1.85 (d, J=5.6 Hz, 1H), 1.82 (s, 1H), 1.80 (s, 3H), 1.71 (s, 3H), 1.64 (d, J=26.8 Hz, 1H), 1.59-1.49 (m, 1H), 1.43 (s, 2H), 1.31-1.20 (m, 1H), 1.14 (s, 2H), 0.96 (q, J=11.9 Hz, 2H).

### Example 310. Preparation of Compound 352b

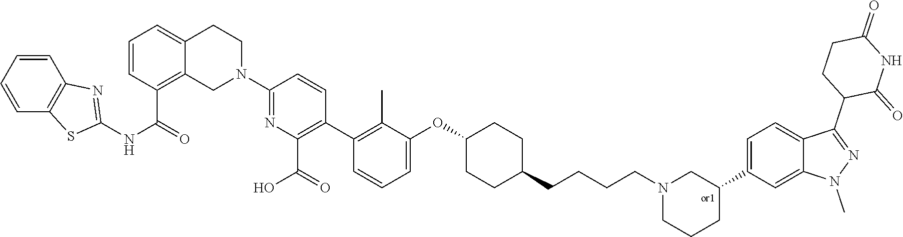 [SMILES: Cc1c(O[C@H]2CC[C@H](CCCCN3CCC[C@@H](c4ccc5c(C6CCC(=O)NC6=O)nn(C)c5c4)C3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1R,4r)-4-(4-((3S)-3-(3-(2,6-dioxopiperidin-3-yl)-1-methyl-1H-indazol-6-yl)piperidin-1-yl)butyl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1R,4r)-4-(4-((3S*)-3-(3-(2,6-dioxopiperidin-3-yl)-1-methyl-1H-indazol-6-yl)piperidin-1-yl)butyl)cyclohexyl)oxy)-2-methylphenyl)picolinic acid]

### Step A. Procedure for Preparation of tert-butyl (3S)-3-(3-(2,6-dioxopiperidin-3-yl)-1-methyl-1H-indazol-6-yl)piperidine-1-carboxylate

A solution of compound tert-butyl (S)-3-(3-(2,6-bis(benzyloxy)pyridin-3-yl)-1-methyl-1H-indazol-6-yl)piperidine-1-carboxylate (13.0 g, 21.5 mmol, 1.00 equiv.), Pd/C (6.50 g, 6.11 mmol, 10.0% purity), and Pd(OH)₂ (6.50 g, 9.26 mmol, 20.0% purity) in EtOH (130 mL) and THE (130 mL)under Ar was degassed under vacuum and purged with H₂ three times. The mixture was stirred under H₂ (50 psi) at 50° C. for 2 hrs. The reaction mixture was filtered, washed with ethyl acetate (300 mL), and concentrated under reduced pressure to give compound tert-butyl (3S)-3-(3-(2,6-dioxopiperidin-3-yl)-1-methyl-1H-indazol-6-yl)piperidine-1-carboxylate (6.13 g, 14.3 mmol, 66.5% yield) as a white solid. MS (ESI) m/z=427.2 [M+H]⁺

### Step B. Procedure for Preparation of 3-(1-methyl-6-((S)-piperidin-3-yl)-1H-indazol-3-yl)piperidine-2,6-dione

A mixture of compound tert-butyl (3S)-3-(3-(2,6-dioxopiperidin-3-yl)-1-methyl-1H-indazol-6-yl)piperidine-1-carboxylate (5.00 g, 11.7 mmol, 1.00 eq) in HCl/dioxane (50.0 mL) was stirred at 25° C. for 1 hr. The reaction mixture was filtered and concentrated under reduced pressure to give 3-(1-methyl-6-((S)-piperidin-3-yl)-1H-indazol-3-yl)piperidine-2,6-dione (6.57 g, 20.0 mmol, 100% yield, HCl) as light-yellow solid. ¹H NMR: (400 MHz, DMSO-d₆) δ 10.89 (s, 1H), 9.60-9.17 (m, 2H), 7.67 (d, J=8.3 Hz, 1H), 7.52 (s, 1H), 7.06 (br d, J=8.4 Hz, 1H), 4.35 (dd, J=5.0, 9.9 Hz, 1H), 3.98 (s, 3H), 3.30 (br d, J=10.6 Hz, 2H), 2.89 (br d, J=3.0 Hz, 1H), 2.74-2.54 (m, 2H), 2.43-2.27 (m, 1H), 2.20-2.10 (m, 1H), 1.98-1.84 (m, 3H), 1.80 (br dd, J=9.0, 14.8 Hz, 1H).

MS (ESI) m/z=327.1 [M+H]⁺.

### Step C. Procedure for Preparation of tert-butyl 6-(8-(benzo[d]thiazol-2-ylcarbamoyl)-3,4-dihydroisoquinolin-2(1H)-yl)-3-(3-(((1R,4r)-4-(4-((3S)-3-(3-(2,6-dioxopiperidin-3-yl)-1-methyl-1H-indazol-6-yl)piperidin-1-yl)butyl)cyclohexyl)oxy)-2-methylphenyl)picolinate

tert-Butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate (60 mg, 1 equiv. 81 μmol) and 3-(1-methyl-6-((R)-piperidin-3-yl)-1H-indazol-3-yl)piperidine-2,6-dione, HCl (35 mg, 1.2 equiv. 97 mol) were suspended in DCM (5 mL). To the mixture was added sodium triacetoxyborohydride (51 mg, 36 μL, 3 equiv. 0.24 mmol). The mixture was stirred at room temperature overnight. The mixture was then concentrated and extracted with DCM and water. The organic layer was separated, washed with brine, dried over Na₂SO₄, filtered, and evaporated to dryness to afford tert-butyl 6-(8-(benzo[d]thiazol-2-ylcarbamoyl)-3,4-dihydroisoquinolin-2(1H)-yl)-3-(3-(((1R,4r)-4-(4-((3S)-3-(3-(2,6-dioxopiperidin-3-yl)-1-methyl-1H-indazol-6-yl)piperidin-1-yl)butyl)cyclohexyl)oxy)-2-methylphenyl)picolinate, which was carried forward without further purification. MS (ESI) m/z: 528.5 [M+2H]²⁺.

### Step D. Procedure for Preparation of 6-(8-(benzo[d]thiazol-2-ylcarbamoyl)-3,4-dihydroisoquinolin-2(1H)-yl)-3-(3-(((1R,4r)-4-(4-((3S)-3-(3-(2,6-dioxopiperidin-3-yl)-1-methyl-1H-indazol-6-yl)piperidin [6-(8-(benzo[d]thiazol-2-ylcarbamoyl)-3,4-dihydroisoquinolin-2(1H)-yl)-3-(3-(((1R,4r)-4-(4-((3S*)-3-(3-(2,6-dioxopiperidin-3-yl)-1-methyl-1H-indazol-6-yl)piperidin-1-yl)butyl)cyclohexyl)oxy)-2-methylphenyl)picolinic acid]

tert-Butyl 6-(8-(benzo[d]thiazol-2-ylcarbamoyl)-3,4-dihydroisoquinolin-2(1H)-yl)-3-(3-(((1R,4r)-4-(4-((3S)-3-(3-(2,6-dioxopiperidin-3-yl)-1-methyl-1H-indazol-6-yl)piperidin-1-yl)butyl)cyclohexyl)oxy)-2-methylphenyl)picolinate (85 mg, 1 equiv. 81 μmol) was dissolved in DCM (4 mL). To the mixture was added TFA (2 mL). The mixture was stirred at room temperature overnight. The reaction mixture was concentrated, and then purified by RP-HPLC to afford 6-(8-(benzo[d]thiazol-2-ylcarbamoyl)-3,4-dihydroisoquinolin-2(1H)-yl)-3-(3-(((1R,4r)-4-(4-((3S)-3-(3-(2,6-dioxopiperidin-3-yl)-1-methyl-1H-indazol-6-yl)piperidin [6-(8-(benzo[d]thiazol-2-ylcarbamoyl)-3,4-dihydroisoquinolin-2(1H)-yl)-3-(3-(((1R,4r)-4-(4-((3S*)-3-(3-(2,6-dioxopiperidin-3-yl)-1-methyl-1H-indazol-6-yl)piperidin-1-yl)butyl)cyclohexyl)oxy)-2-methylphenyl)picolinic acid] (35 mg, 35 μmol, 43%) as a white solid. MS (ESI) m/z: 500.4 [M+2H]²⁺; ¹H NMR (400 MHz, DMSO-d₆) δ 10.80 (s, 1H), 7.95 (d, J=7.9 Hz, 1H), 7.72 (d, J=8.0 Hz, 1H), 7.56 (dd, J=8.3, 2.6 Hz, 2H), 7.39 (ddd, J=14.6, 8.5, 2.7 Hz, 4H), 7.29 (dt, J=10.6, 7.6 Hz, 2H), 7.03-6.93 (m, 2H), 6.87 (d, J=8.8 Hz, 1H), 6.83 (d, J=8.3 Hz, 1H), 6.55 (d, J=7.5 Hz, 1H), 4.91 (s, 2H), 4.26 (dd, J=9.8, 5.0 Hz, 1H), 3.90 (d, J=3.1 Hz, 3H), 3.84 (t, J=5.9 Hz, 2H), 3.04 (s, 2H), 2.95 (t, J=6.0 Hz, 2H), 2.89 (s, 1H), 2.67-2.45 (m, 2H), 2.34-2.21 (m, 1H), 2.15-2.04 (m, 1H), 2.03-1.96 (m, 2H), 1.85 (d, J=5.5 Hz, 1H), 1.82 (s, 1H), 1.80 (s, 3H), 1.73-1.49 (m, 3H), 1.43 (s, 1H), 1.31-1.20 (m, 1H), 1.13 (d, J=10.0 Hz, 3H), 0.96 (q, J=12.0 Hz, 2H).

### Example 311. Preparation of Compound 354a

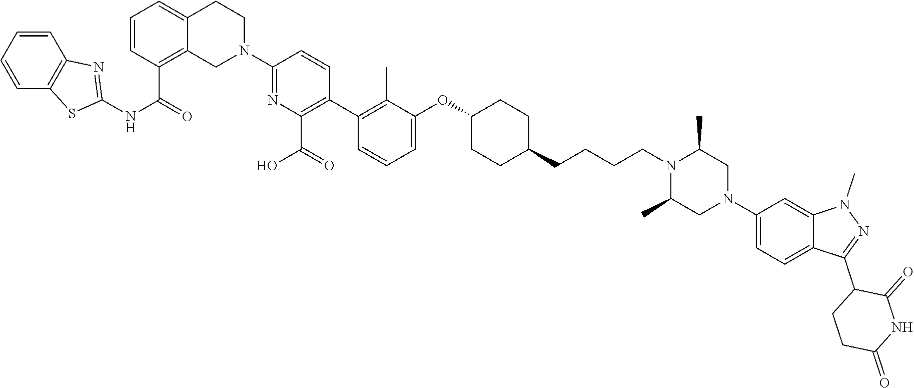 [SMILES: Cc1c(O[C@H]2CC[C@H](CCCCN3[C@H](C)CN(c4ccc5c(C6CCC(=O)NC6=O)nn(C)c5c4)C[C@@H]3C)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-(4-((2S,6R)-4-(3-(2,6-dioxopiperidin-3-yl)-1-methyl-1H-indazol-6-yl)-2,6-dimethylpiperazin-1-yl)butyl)cyclohexyl)oxy)-2-methylphenyl)picolinic acid

### Step A. Preparation of tert-butyl (2S,6R)-4-(3-(2,6-bis(benzyloxy)pyridin-3-yl)-1-methyl-1H-indazol-6-yl)-2,6-dimethylpiperazine-1-carboxylate

3-(2,6-Bis(benzyloxy)pyridin-3-yl)-6-bromo-1-methyl-1H-indazole (1.0 g, 1 equiv. 2.0 mmol) was dissolved in 1,4-dioxane (10 mL). To the mixture was added tert-butyl (2S,6R)-2,6-dimethylpiperazine-1-carboxylate (0.51 g, 1.2 equiv. 2.4 mmol), followed by (2-dicyclohexylphosphino-2′,6′-diisopropoxy-1,1′-biphenyl)[2-(2′-amino-1,1′-biphenyl)]palladium(II) methanesulfonate (0.17 g, 0.1 equiv. 0.20 mmol), and sodium tert-butoxide (0.58 g, 3.0 mL, 2 molar, 3 equiv. 6.0 mmol). The mixture was then purged with N₂, and then heated at 100° C. for ˜2 hrs. The mixture was cooled down to room temperature, quenched with water, and then extracted with ethyl acetate (3×). The organic layers were combined, washed with brine, dried over Na₂SO₄, filtered, and evaporated to dryness. The residue was then purified by silica gel column chromatography (0-100% ethyl acetate in heptane) to afford tert-butyl (2S,6R)-4-(3-(2,6-bis(benzyloxy)pyridin-3-yl)-1-methyl-1H-indazol-6-yl)-2,6-dimethylpiperazine-1-carboxylate, which was used in the next step without further purification.

### Step B. Preparation of tert-butyl (2S,6R)-4-(3-(2,6-dioxopiperidin-3-yl)-1-methyl-1H-indazol-6-yl)-2,6-dimethylpiperazine-1-carboxylate

tert-butyl (2S,6R)-4-(3-(2,6-bis(benzyloxy)pyridin-3-yl)-1-methyl-1H-indazol-6-yl)-2,6-dimethylpiperazine-1-carboxylate (1.3 g, 1 equiv. 2.0 mmol) was dissolved in ethyl acetate (20 mL). To the mixture was added Pd(OH)₂ (0.35 g, 20% Wt, 0.25 equiv. 0.50 mmol). The mixture was stirred at 50° C. under H₂ overnight. The mixture was filtered through celite and rinsed with ethyl acetate (3×). The filtrate was then evaporated to dryness. The residue was then purified by RP-HPLC to afford tert-butyl (2S,6R)-4-(3-(2,6-dioxopiperidin-3-yl)-1-methyl-1H-indazol-6-yl)-2,6-dimethylpiperazine-1-carboxylate (0.56 g, 1.2 mmol, 61%) as a white solid. MS (ESI) m/z: 456.4 [M+H]⁺.

### Step C. Preparation of 3-(6-((3S,5R)-3,5-dimethylpiperazin-1-yl)-1-methyl-1H-indazol-3-yl)piperidine-2,6-dione, HCl

tert-butyl (2S,6R)-4-(3-(2,6-dioxopiperidin-3-yl)-1-methyl-1H-indazol-6-yl)-2,6-dimethylpiperazine-1-carboxylate (0.56 g, 1 equiv. 1.2 mmol) was suspended in 4M HCl in dioxane (4 mL). The mixture was then stirred at room temperature overnight. The mixture was concentrated to afford 3-(6-((3S,5R)-3,5-dimethylpiperazin-1-yl)-1-methyl-1H-indazol-3-yl)piperidine-2,6-dione, HCl (0.49 g, 1.3 mmol, 100%) as a white solid. MS (ESI) m/z: 356.2 [M+H]⁺.

### Step D. Preparation of tert-butyl 6-(8-(benzo[d]thiazol-2-ylcarbamoyl)-3,4-dihydroisoquinolin-2(1H)-yl)-3-(3-(((1s,4r)-4-(4-((2S,6R)-4-(3-(2,6-dioxopiperidin-3-yl)-1-methyl-1H-indazol-6-yl)-2,6-dimethylpiperazin-1-yl)butyl)cyclohexyl)oxy)-2-methylphenyl)picolinate

tert-Butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate (60 mg, 1 equiv. 81 μmol) and 3-(6-((3S,5R)-3,5-dimethylpiperazin-1-yl)-1-methyl-1H-indazol-3-yl)piperidine-2,6-dione, HCl (35 mg, 1.1 equiv. 89 μmol) was suspended in DCM (5 mL). To the mixture was added sodium triacetoxyborohydride (51 mg, 36 μL, 3 equiv. 0.24 mmol). The mixture was stirred at room temperature overnight. The mixture was then concentrated and extracted with DCM and water. The organic layer was separated, washed with brine, dried over Na₂SO₄, filtered, and evaporated to dryness to afford tert-butyl 6-(8-(benzo[d]thiazol-2-ylcarbamoyl)-3,4-dihydroisoquinolin-2(1H)-yl)-3-(3-(((1s,4r)-4-(4-((2S,6R)-4-(3-(2,6-dioxopiperidin-3-yl)-1-methyl-1H-indazol-6-yl)-2,6-dimethylpiperazin-1-yl)butyl)cyclohexyl)oxy)-2-methylphenyl)picolinate, which was carried forward without further purification. MS (ESI) m/z: 543.0 [M+2H]²⁺.

### Step E. Preparation of 6-(8-(benzo[d]thiazol-2-ylcarbamoyl)-3,4-dihydroisoquinolin-2(1H)-yl)-3-(3-(((1s,4r)-4-(4-((2S,6R)-4-(3-(2,6-dioxopiperidin-3-yl)-1-methyl-1H-indazol-6-yl)-2,6-dimethylpiperazin-1-yl)butyl)cyclohexyl)oxy)-2-methylphenyl)picolinic acid

tert-butyl 6-(8-(benzo[d]thiazol-2-ylcarbamoyl)-3,4-dihydroisoquinolin-2(1H)-yl)-3-(3-(((1s,4r)-4-(4-((2S,6R)-4-(3-(2,6-dioxopiperidin-3-yl)-1-methyl-1H-indazol-6-yl)-2,6-dimethylpiperazin-1-yl)butyl)cyclohexyl)oxy)-2-methylphenyl)picolinate (88 mg, 1 equiv. 81 μmol) was dissolved in DCM (4 mL). To the mixture was added 1 mL TFA. The mixture was stirred at room temperature. The mixture was concentrated, and purified by RP-HPLC to afford 6-(8-(benzo[d]thiazol-2-ylcarbamoyl)-3,4-dihydroisoquinolin-2(1H)-yl)-3-(3-(((1s,4r)-4-(4-((2S,6R)-4-(3-(2,6-dioxopiperidin-3-yl)-1-methyl-1H-indazol-6-yl)-2,6-dimethylpiperazin-1-yl)butyl)cyclohexyl)oxy)-2-methylphenyl)picolinic acid (9 mg, 9 μmol, 10%) as a white solid.

MS (ESI) m/z: 514.9 [M+2H]²⁺; ¹H NMR (400 MHz, DMSO-d₆) δ 12.79 (s, 1H), 10.77 (s, 1H), 7.96 (d, J=7.8 Hz, 1H), 7.72 (d, J=8.1 Hz, 1H), 7.55 (d, J=7.4 Hz, 1H), 7.44-7.35 (m, 3H), 7.35-7.24 (m, 2H), 7.00 (t, J=7.8 Hz, 1H), 6.87 (dd, J=17.1, 8.6 Hz, 3H), 6.75 (s, 1H), 6.54 (d, J=7.4 Hz, 1H), 4.91 (s, 2H), 3.86 (d, J=5.9 Hz, 1H), 3.83 (s, 4H), 3.57 (s, 2H), 2.96 (t, J=6.0 Hz, 2H), 2.58-2.45 (m, 2H), 2.22 (s, 1H), 2.09 (dd, J=13.0, 5.9 Hz, 1H), 1.85 (d, J=6.9 Hz, 1H), 1.80 (s, 3H), 1.71 (d, J=12.4 Hz, 2H), 1.37 (s, 1H), 1.29 (s, 8H), 1.17 (s, 5H), 1.01 (s, 6H).

### Example 312. Preparation of Compound 354b

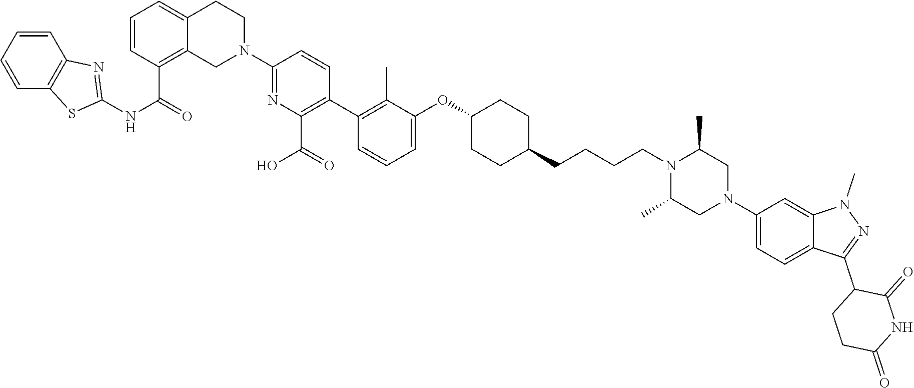 [SMILES: Cc1c(O[C@H]2CC[C@H](CCCCN3[C@@H](C)CN(c4ccc5c(C6CCC(=O)NC6=O)nn(C)c5c4)C[C@@H]3C)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1r,4r)-4-(4-((2S,6S)-4-(3-(2,6-dioxopiperidin-3-yl)-1-methyl-1H-indazol-6-yl)-2,6-dimethylpiperazin-1-yl)butyl)cyclohexyl)oxy)-2-methylphenyl)picolinic acid

### Step A. Preparation of tert-butyl (2S,6S)-4-(3-(2,6-bis(benzyloxy)pyridin-3-yl)-1-methyl-1H-indazol-6-yl)-2,6-dimethylpiperazine-1-carboxylate

3-(2,6-bis(benzyloxy)pyridin-3-yl)-6-bromo-1-methyl-1H-indazole (1.0 g, 1 equiv. 2.0 mmol) was dissolved in 1,4-dioxane (10 mL). To the mixture was added tert-butyl (2S,6S)-2,6-dimethylpiperazine-1-carboxylate (0.51 g, 1.2 equiv. 2.4 mmol), followed by (2-dicyclohexylphosphino-2′,6′-diisopropoxy-1,1′-biphenyl)[2-(2′-amino-1,1′-biphenyl)]palladium(II) methanesulfonate (0.17 g, 0.1 equiv. 0.20 mmol), and sodium tert-butoxide (0.58 g, 3.0 mL, 2 molar, 3 equiv. 6.0 mmol). The mixture was then purged with N₂, and then heated at 100° C. for ˜2 hrs. The mixture was cooled down to room temperature, quenched with water, and then extracted with ethyl acetate (3×). The organic layers were combined, washed with brine, dried over Na₂SO₄, filtered, and evaporated to dryness. The residue was then purified by silica gel column chromatography (0-100% ethyl acetate in heptane) to afford tert-butyl (2S,6S)-4-(3-(2,6-bis(benzyloxy)pyridin-3-yl)-1-methyl-1H-indazol-6-yl)-2,6-dimethylpiperazine-1-carboxylate, which was used in the next step without further purification.

### Step B. Preparation of tert-butyl (2S,6S)-4-(3-(2,6-dioxopiperidin-3-yl)-1-methyl-1H-indazol-6-yl)-2,6-dimethylpiperazine-1-carboxylate

tert-Butyl (2S,6S)-4-(3-(2,6-bis(benzyloxy)pyridin-3-yl)-1-methyl-1H-indazol-6-yl)-2,6-dimethylpiperazine-1-carboxylate (1.3 g, 1 equiv. 2.0 mmol) was dissolved in ethyl acetate (20 mL). To the mixture was added Pd(OH)₂ (0.35 g, 20% Wt, 0.25 equiv. 0.50 mmol). The mixture was stirred at 50° C. under H₂ overnight. The mixture was filtered through celite and rinsed with ethyl acetate (3×). The filtrate was then evaporated to dryness. The residue was then purified by RP-HPLC to afford tert-butyl (2S,6S)-4-(3-(2,6-dioxopiperidin-3-yl)-1-methyl-1H-indazol-6-yl)-2,6-dimethylpiperazine-1-carboxylate (415 mg, 911 μmol, 46%) as a white solid. MS (ESI) m/z: 456.3 [M+H]⁺.

### Step C. Preparation of 3-(6-((3S,5S)-3,5-dimethylpiperazin-1-yl)-1-methyl-1H-indazol-3-yl)piperidine-2,6-dione, HCl

tert-Butyl (2S,6S)-4-(3-(2,6-dioxopiperidin-3-yl)-1-methyl-1H-indazol-6-yl)-2,6-dimethylpiperazine-1-carboxylate (415 mg, 1 equiv. 911 μmol) was suspended in 4M HCl in dioxane (4 mL). The mixture was then stirred at room temperature overnight. The mixture was concentrated to afford 3-(6-((3S,5S)-3,5-dimethylpiperazin-1-yl)-1-methyl-1H-indazol-3-yl)piperidine-2,6-dione, HCl (275 mg, 702 μmol, 77.0%) as an off-white solid. MS (ESI) m/z: 356.2 [M+H]⁺.

### Step D. Preparation of tert-butyl 6-(8-(benzo[d]thiazol-2-ylcarbamoyl)-3,4-dihydroisoquinolin-2(1H)-yl)-3-(3-(((1r,4r)-4-(4-((2S,6S)-4-(3-(2,6-dioxopiperidin-3-yl)-1-methyl-1H-indazol-6-yl)-2,6-dimethylpiperazin-1-yl)butyl)cyclohexyl)oxy)-2-methylphenyl)picolinate

tert-Butyl 6-(8-(benzo[d]thiazol-2-ylcarbamoyl)-3,4-dihydroisoquinolin-2(1H)-yl)-3-(2-methyl-3-(((1s,4r)-4-(4-oxobutyl)cyclohexyl)oxy)phenyl)picolinate (60 mg, 1 equiv. 81 μmol) and 3-(6-((3S,5S)-3,5-dimethylpiperazin-1-yl)-1-methyl-1H-indazol-3-yl)piperidine-2,6-dione, HCl (35 mg, 1.1 equiv. 89 μmol) was suspended in DCM (5 mL). To the mixture was added sodium triacetoxyborohydride (51 mg, 36 μL, 3 equiv. 0.24 mmol). The mixture was stirred at room temperature overnight. The mixture was then concentrated and extracted with DCM and water. The organic layer was separated, washed with brine, dried over Na₂SO₄, filtered, and evaporated to dryness to afford tert-butyl 6-(8-(benzo[d]thiazol-2-ylcarbamoyl)-3,4-dihydroisoquinolin-2(1H)-yl)-3-(3-(((1r,4r)-4-(4-((2S,6S)-4-(3-(2,6-dioxopiperidin-3-yl)-1-methyl-1H-indazol-6-yl)-2,6-dimethylpiperazin-1-yl)butyl)cyclohexyl)oxy)-2-methylphenyl)picolinate, which was carried forward without further purification. MS (ESI) m/z: 542.9 [M+2H]²⁺.

### Step E. Preparation of 6-(8-(benzo[d]thiazol-2-ylcarbamoyl)-3,4-dihydroisoquinolin-2(1H)-yl)-3-(3-(((1r,4r)-4-(4-((2S,6S)-4-(3-(2,6-dioxopiperidin-3-yl)-1-methyl-1H-indazol-6-yl)-2,6-dimethylpiperazin-1-yl)butyl)cyclohexyl)oxy)-2-methylphenyl)picolinic acid

tert-Butyl 6-(8-(benzo[d]thiazol-2-ylcarbamoyl)-3,4-dihydroisoquinolin-2(1H)-yl)-3-(3-(((1r,4r)-4-(4-((2S,6S)-4-(3-(2,6-dioxopiperidin-3-yl)-1-methyl-1H-indazol-6-yl)-2,6-dimethylpiperazin-1-yl)butyl)cyclohexyl)oxy)-2-methylphenyl)picolinate (88 mg, 1 equiv. 81 μmol) was dissolved in DCM (4 mL). To the mixture was added TFA (1 mL). The mixture was stirred at room temperature. The mixture was concentrated, and purified by RP-HPLC to afford 6-(8-(benzo[d]thiazol-2-ylcarbamoyl)-3,4-dihydroisoquinolin-2(1H)-yl)-3-(3-(((1r,4r)-4-(4-((2S,6S)-4-(3-(2,6-dioxopiperidin-3-yl)-1-methyl-1H-indazol-6-yl)-2,6-dimethylpiperazin-1-yl)butyl)cyclohexyl)oxy)-2-methylphenyl)picolinic acid (28 mg, 27 μmol, 34%) as a white solid.

MS (ESI) m/z: 514.9 [M+2H]²⁺; ¹H NMR (400 MHz, DMSO-d₆) δ 12.78 (s, 1H), 10.77 (s, 1H), 7.96 (d, J=7.9 Hz, 1H), 7.72 (d, J=8.0 Hz, 1H), 7.55 (d, J=7.5 Hz, 1H), 7.47-7.24 (m, 5H), 7.00 (t, J=7.9 Hz, 1H), 6.92-6.77 (m, 4H), 6.54 (d, J=7.4 Hz, 1H), 4.91 (s, 2H), 4.19 (dd, J=9.2, 5.1 Hz, 1H), 4.13 (s, 1H), 3.85 (d, J=5.9 Hz, 1H), 3.83 (s, 3H), 2.96 (t, J=6.0 Hz, 2H), 2.85 (s, 9H), 2.54 (p, J=7.3 Hz, 2H), 2.27-2.19 (m, 1H), 2.15-1.98 (m, 3H), 1.80 (s, 2H), 1.72 (d, J=12.4 Hz, 2H), 1.44 (s, 3H), 1.28 (s, 5H), 1.18 (s, 3H), 1.09 (s, 5H), 0.99 (q, J=15.0 Hz, 2H).

### Example 313. Preparation of Compound 333b

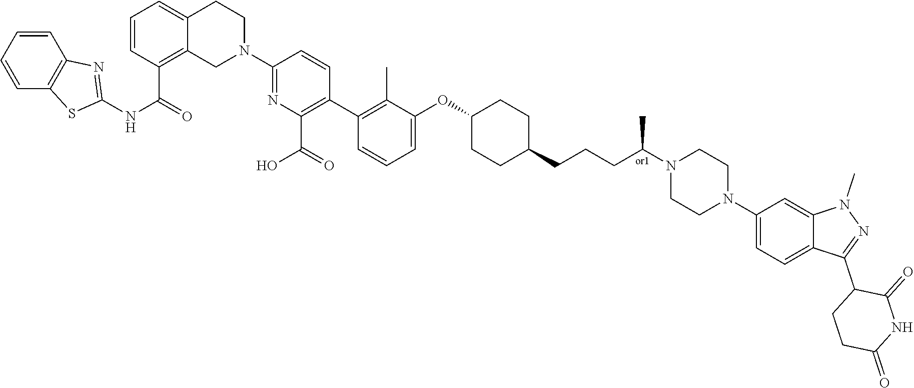 [SMILES: Cc1c(O[C@H]2CC[C@H](CCC[C@@H](C)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1H)-yl)-3-(3-(((1s,4r)-4-((4R)-4-(4-(3-(2,6-dioxopiperidin-3-yl)-1-methyl-1H-indazol-6-yl)piperazin-1-yl)pentyl)cyclohexyl)oxy)-2-methylphenyl)picolinic acid

### [6-(8-(benzo[d]thiazol-2-ylcarbamoyl)-3,4-dihydroisoquinolin-2(1H)-yl)-3-(3-(((1S,4r)-4-((4R*)-4-(4-(3-(2,6-dioxopiperidin-3-yl)-1-methyl-1H-indazol-6-yl)piperazin-1-yl)pentyl)cyclohexyl)oxy)-2-methylphenyl)picolinic acid]

### Step A. Procedure for Preparation of 3-(2,6-dibenzyloxy-3-pyridyl)-1-methyl-6-piperazin-1-yl-indazole

A mixture of 6-bromo-3-(2,6-dibenzyloxy-3-pyridyl)-1-methyl-indazole (2.5 g, 5.00 mmol, 1.0 equiv.), piperazine (1.29 g, 14.99 mmol, 3.0 equiv.), Pd₂(dba)₃ (457.51 mg, 499.61 μmol, 0.1 equiv.), NaOtBu (960.26 mg, 9.99 mmol, 2.0 equiv.), and tritertbutylphosphane (2.02 g, 999.23 mol, 2.35 mL, 10% purity, 0.2 equiv.) in toluene (80 mL) was degassed and purged with N₂ three times. The mixture was stirred at 100° C. for 16 hours under N₂ atmosphere. The reaction mixture was concentrated under reduced pressure to give a residue. The residue was purified by flash silica gel chromatography (10˜20% MeOH/DCM) to give 3-(2,6-dibenzyloxy-3-pyridyl)-1-methyl-6-piperazin-1-yl-indazole (2.4 g, 4.67 mmol, 93.44% yield) as a yellow oil. MS (ESI) m/z: 506.4 [M+H]⁺.

### Step B. Procedure for Preparation of 3-(2,6-bis(benzyloxy)pyridin-3-yl)-6-(4-(5-((1r,4s)-4-(3-bromo-2-methylphenoxy)cyclohexyl)pentan-2-yl)piperazin-1-yl)-1-methyl-1H-indazole

A solution of 5-((1r,4s)-4-(3-bromo-2-methylphenoxy)cyclohexyl)pentan-2-one (1.5 g, 4.25 mmol, 1.0 equiv.), 3-(2,6-dibenzyloxy-3-pyridyl)-1-methyl-6-piperazin-1-yl-indazole (2.15 g, 4.25 mmol, 1.0 equiv.) in DCM (15 mL) was stirred at 35° C. for 1 hour. To the mixture was added NaBH(OAc)₃ (2.70 g, 12.74 mmol, 3 equiv.). The resulting mixture was stirred at 35° C. for 11 hours. DCM (300 mL) and water (300 mL) were added, and layers were separated. The aqueous layer was extracted with DCM (50 mL×2), dried over anhydrous sodium sulfate, filtered, and concentrated under vacuum. The residue was purified by flash silica gel chromatography (0˜50% ethyl acetate/petroleum ether) to give 3-(2,6-bis(benzyloxy)pyridin-3-yl)-6-(4-(5-((1r,4s)-4-(3-bromo-2-methylphenoxy)cyclohexyl)pentan-2-yl)piperazin-1-yl)-1-methyl-1H-indazole (1.8 g, 2.06 mmol, 48.60% yield) as a yellow solid. MS (ESI) m/z: 844.6 [M+H]⁺

### Step C. Procedure for Preparation of 3-(2,6-bis(benzyloxy)pyridin-3-yl)-6-(4-((S)-5-((1r,4r)-4-(3-bromo-2-methylphenoxy)cyclohexyl)pentan-2-yl)piperazin-1-yl)-1-methyl-1H-indazole and 3-(2,6-bis(benzyloxy)pyridin-3-yl)-6-(4-((R)-5-((1r,4s)-4-(3-bromo-2-methylphenoxy)cyclohexyl)pentan-2-yl)piperazin-1-yl)-1-methyl-1H-indazole

The stereoisomers of 3-(2,6-bis(benzyloxy)pyridin-3-yl)-6-(4-(5-((1r,4s)-4-(3-bromo-2-methylphenoxy)cyclohexyl)pentan-2-yl)piperazin-1-yl)-1-methyl-1H-indazole (1.0 g, 949.11 mol, 1.0 equiv.) were separated by SFC to give 3-(2,6-bis(benzyloxy)pyridin-3-yl)-6-(4-((S)-5-((1r,4r)-4-(3-bromo-2-methylphenoxy)cyclohexyl)pentan-2-yl)piperazin-1-yl)-1-methyl-1H-indazole (150 mg, 163.01 μmol, 12.90% yield) and 3-(2,6-bis(benzyloxy)pyridin-3-yl)-6-(4-((R)-5-((1r,4s)-4-(3-bromo-2-methylphenoxy)cyclohexyl)pentan-2-yl)piperazin-1-yl)-1-methyl-1H-indazole (240 mg, 276.17 μmol, 22.85% yield) as yellow solids. MS (ESI) m/z: 844.3 [M+H]⁺.

MS (ESI) m/z: 844.3 [M+H]⁺

### Step D. Procedure for Preparation of 3-(2,6-bis(benzyloxy)pyridin-3-yl)-1-methyl-6-(4-((R)-5-((1r,4S)-4-(2-methyl-3-(4,4,5,5-tetramethyl-1,3,2-dioxaborolan-2-yl)phenoxy)cyclohexyl)pentan-2-yl)piperazin-1-yl)-1H-indazole

A mixture of 3-(2,6-bis(benzyloxy)pyridin-3-yl)-6-(4-((R)-5-((1r,4S)-4-(3-bromo-2-methylphenoxy)cyclohexyl)pentan-2-yl)piperazin-1-yl)-1-methyl-1H-indazole (240 mg, 284.73 mol, 1.0 equiv.), 4,4,4′,4′,5,5,5′,5′-octamethyl-2,2′-bi(1,3,2-dioxaborolane) (216.91 mg, 854.19 mol, 3.0 equiv.) and KOAc (83.83 mg, 854.19 μmol, 3.0 equiv.) in dioxane (4 mL) was degassed and purged with N₂ three times. Pd(dppf)Cl₂ (41.67 mg, 56.95 μmol, 0.2 equiv.) was then added into the mixture. The reaction mixture was stirred at 90° C. for 12 hours under N₂ atmosphere. Ethyl acetate (100 mL) and water (100 mL) were added, and the layers were separated. The aqueous layer was extracted with ethyl acetate (30 mL×2), dried over anhydrous sodium sulfate, filtered, and concentrated under vacuum. The residue was purified by prep-TLC (SiO₂, petroleum ether:ethyl acetate=1:1) to give 3-(2,6-bis(benzyloxy)pyridin-3-yl)-1-methyl-6-(4-((R)-5-((1r,4S)-4-(2-methyl-3-(4,4,5,5-tetramethyl-1,3,2-dioxaborolan-2-yl)phenoxy)cyclohexyl)pentan-2-yl)piperazin-1-yl)-1H-indazole (90 mg, 93.04 μmol, 32.68% yield) as a yellow solid. MS (ESI) m/z: 890.4 [M+H]³⁰

### Step E. Procedure for Preparation of 3-(1-methyl-6-(4-((R)-5-((1r,4s)-4-(2-methyl-3-(4,4,5,5-tetramethyl-1,3,2-dioxaborolan-2-yl)phenoxy)cyclohexyl)pentan-2-yl)piperazin-1-yl)-1H-indazol-3-yl)piperidine-2,6-dione

To a mixture of 3-(2,6-bis(benzyloxy)pyridin-3-yl)-1-methyl-6-(4-((R)-5-((1r,4s)-4-(2-methyl-3-(4,4,5,5-tetramethyl-1,3,2-dioxaborolan-2-yl)phenoxy)cyclohexyl)pentan-2-yl)piperazin-1-yl)-1H-indazole (90 mg, 101.13 μmol, 1.0 equiv.) in THE (2 mL) and EtOH (2 mL) was added Pd/C (30.00 mg, 28.19 μmol, 10% purity) and Pd(OH)₂ (30.00 mg, 42.72 μmol, 20% purity) under H₂ atmosphere (15 Psi). The mixture was stirred at 40° C. for 12 hours. The reaction mixture was filtered and concentrated under reduced pressure to give a filter cake with a little EtOH. The filtrate was concentrated under reduced pressure to give 3-(1-methyl-6-(4-((R)-5-((1r,4s)-4-(2-methyl-3-(4,4,5,5-tetramethyl-1,3,2-dioxaborolan-2-yl)phenoxy)cyclohexyl)pentan-2-yl)piperazin-1-yl)-1H-indazol-3-yl)piperidine-2,6-dione (85 mg, 83.60 μmol, 82.67% yield) as a yellow solid. MS (ESI) m/z: 712.4 [M+H]⁺

### Step F. Procedure for Preparation of tert-butyl 6-(8-(benzo[d]thiazol-2-ylcarbamoyl)-3,4-dihydroisoquinolin-2(1H)-yl)-3-(3-(((1s,4r)-4-((4R)-4-(4-(3-(2,6-dioxopiperidin-3-yl)-1-methyl-1H-indazol-6-yl)piperazin-1-yl)pentyl)cyclohexyl)oxy)-2-methylphenyl)picolinate

A mixture of 3-(1-methyl-6-(4-((R)-5-((1r,4s)-4-(2-methyl-3-(4,4,5,5-tetramethyl-1,3,2-dioxaborolan-2-yl)phenoxy)cyclohexyl)pentan-2-yl)piperazin-1-yl)-1H-indazol-3-yl)piperidine-2,6-dione (85 mg, 83.60 μmol, 1.0 equiv.), tert-butyl 6-(8-(benzo[d]thiazol-2-ylcarbamoyl)-3,4-dihydroisoquinolin-2(1H)-yl)-3-bromopicolinate (56.73 mg, 100.32 μmol, 1.2 equiv.), KF (14.57 mg, 250.79 μmol, 5.88 μL, 3.0 equiv.), and H₂O (0.2 mL) in dioxane (2 mL) was degassed and purged with N₂ three times. Then Ad₂nBuP Pd G₃ (12.18 mg, 16.72 μmol, 0.2 equiv.) was added into the mixture. The resulting mixture was degassed and purged with N₂ three times. The mixture was stirred at 100° C. for 12 hours under N₂ atmosphere. The reaction mixture was concentrated under reduced pressure to give a residue. The residue was purified by prep-TLC (SiO₂, DCM:MeOH=20:1) to give tert-butyl 6-(8-(benzo[d]thiazol-2-ylcarbamoyl)-3,4-dihydroisoquinolin-2(1H)-yl)-3-(3-(((1s,4r)-4-((4R)-4-(4-(3-(2,6-dioxopiperidin-3-yl)-1-methyl-1H-indazol-6-yl)piperazin-1-yl)pentyl)cyclohexyl)oxy)-2-methylphenyl)picolinate (60 mg, 52.13 μmol, 62.36% yield) as a yellow solid. MS (ESI) m/z: 1070.5 [M+H]⁺.

### Step G. Procedure for Preparation of 6-(8-(benzo[d]thiazol-2-ylcarbamoyl)-3,4-dihydroisoquinolin-2(1H)-yl)-3-(3-(((1s,4r)-4-((4R)-4-(4-(3-(2,6-dioxopiperidin-3-yl)-1-methyl-1H-indazol-6-yl)piperazin-1-yl)pentyl)cyclohexyl)oxy)-2-methylphenyl)picolinic acid

A solution of tert-butyl 6-(8-(benzo[d]thiazol-2-ylcarbamoyl)-3,4-dihydroisoquinolin-2(1H)-yl)-3-(3-(((1s,4r)-4-((4R)-4-(4-(3-(2,6-dioxopiperidin-3-yl)-1-methyl-1H-indazol-6-yl)piperazin-1-yl)pentyl)cyclohexyl)oxy)-2-methylphenyl)picolinate (60 mg, 56.06 μmol, 1.0 equiv.) in TFA (2 mL) and DCM (1 mL) was stirred at 40° C. for 1 hour. The reaction mixture was concentrated under reduced pressure to give a residue. The residue was purified by prep-HPLC to give 6-(8-(benzo[d]thiazol-2-ylcarbamoyl)-3,4-dihydroisoquinolin-2(1H)-yl)-3-(3-(((1s,4r)-4-((4R)-4-(4-(3-(2,6-dioxopiperidin-3-yl)-1-methyl-1H-indazol-6-yl)piperazin-1-yl)pentyl)cyclohexyl)oxy)-2-methylphenyl)picolinic acid (15.71 mg, 15.06 μmol, 26.87% yield) as a white solid. MS (ESI) m/z: 1014.4 [M+H]⁺. ¹H NMR (400 MHz, DMSO-d₆) δ=12.90-12.50 (m, 2H), 10.85 (s, 1H), 8.03 (d, J=8.0 Hz, 1H), 7.79 (d, J=7.6 Hz, 1H), 7.62 (d, J=7.6 Hz, 1H), 7.51-7.48 (m, 1H), 7.47-7.43 (m, 3H), 7.40-7.33 (m, 2H), 7.10-7.03 (m, 1H), 6.96 (d, J=8.8 Hz, 1H), 6.91 (d, J=8.4 Hz, 2H), 6.84 (s, 1H), 6.61 (d, J=7.6 Hz, 1H), 4.97 (s, 2H), 4.29-4.23 (m, 1H), 4.22-4.13 (m, 1H), 3.94-3.86 (m, 5H), 3.27-3.12 (m, 4H), 3.02 (t, J=5.6 Hz, 2H), 2.79-2.69 (m, 2H), 2.65-2.55 (m, 4H), 2.32-2.25 (m, 1H), 2.19-2.12 (m, 1H), 2.11-2.04 (m, 2H), 1.86 (s, 3H), 1.82-1.74 (m, 2H), 1.57-1.47 (m, 1H), 1.41-1.27 (m, 6H), 1.23 (s, 3H), 1.11-0.96 (m, 5H).

### Example 314. Preparation of Compound 355

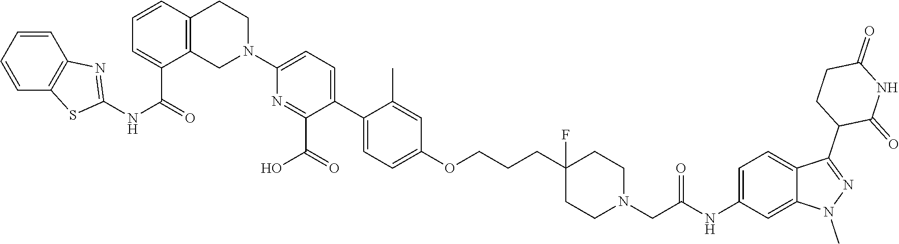 [SMILES: Cc1cc(OCCCC2(F)CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[4-[3-[1-[2-[[3-(2,6-dioxo-3-piperidyl)-1-methyl-indazol-6-yl]amino]-2-oxo-ethyl]-4-fluoro-4-piperidyl]propoxy]-2-methyl-phenyl]pyridine-2-carboxylic acid

### Step A. Procedure for Preparation of tert-butyl 4-fluoro-4-formyl-piperidine-1-carboxylate

To a solution of oxalyl dichloride (2.72 g, 21.43 mmol, 1.88 mL, 2.0 equiv.) in DCM (30 mL) was added dropwise into DMSO (3.35 g, 42.87 mmol, 3.35 mL, 4.0 equiv.) at −78° C. under N₂ atmosphere. The mixture was stirred at −78° C. for 1 hour. After which time tert-butyl 4-fluoro-4-(hydroxymethyl)piperidine-1-carboxylate (2.5 g, 10.72 mmol, 1.0 equiv.) in DCM (30 mL) was added dropwise at −78° C. The solution was stirred for 1 hour at −78° C. Then TEA (6.51 g, 64.30 mmol, 8.95 mL, 6.0 equiv.) was added into the solution. The solution was stirred at −78° C. for 0.5 hours under N₂ atmosphere. The reaction mixture was then partitioned between DCM (100 mL) and water (50 mL). The organic phase was separated, washed with brine (50 mL×3), dried over Na₂SO₄, filtered, and concentrated under reduced pressure to give tert-butyl 4-fluoro-4-formyl-piperidine-1-carboxylate (4.8 g, crude) as a yellow oil. ¹H NMR (400 MHz, DMSO-d₆) δ=3.86 (s, 2H), 3.16-2.76 (m, 2H), 1.86-1.47 (m, 4H), 1.47-1.33 (m, 9H)

### Step B. Procedure for Preparation of tert-butyl 4-[(E)-3-ethoxy-3-oxo-prop-1-enyl]-4-fluoro-piperidine-1-carboxylate

To a solution of NaH (747.13 mg, 18.68 mmol, 60% purity, 1.8 equiv.) in THF (120 mL) was added ethyl 2-diethoxyphosphorylacetate (3.49 g, 15.57 mmol, 3.09 mL, 1.5 equiv.) at 0° C. for 1 hour under N₂ atmosphere. To this solution was added tert-butyl 4-fluoro-4-formyl-piperidine-1-carboxylate (2.4 g, 10.38 mmol, 1 equiv.) in THE (10 mL). The mixture was stirred at 0° C. for 11 hours. The reaction mixture was quenched by addition of aq. NH₄Cl (100 mL) at 0° C., and then extracted with ethyl acetate (200 mL). The combined organic layers were washed with brine 100 mL, dried over Na₂SO₄, filtered, and concentrated under reduced pressure to give a residue. The residue was purified by flash silica gel chromatography (0˜6% ethyl acetate/petroleum ether) to give tert-butyl 4-[(E)-3-ethoxy-3-oxo-prop-1-enyl]-4-fluoro-piperidine-1-carboxylate (1.3 g, 4.31 mmol, 20.78% yield) as a yellow oil.

¹H NMR (400 MHz, DMSO-d₆) δ=7.06-6.89 (m, 1H), 6.02 (d, J=15.6 Hz, 1H), 4.22-4.04 (m, 2H), 3.86 (d, J=10.4 Hz, 2H), 2.98 (s, 2H), 1.96-1.66 (m, 4H), 1.43-1.38 (m, 9H), 1.27-1.14 (m, 3H)

### Step C. Procedure for Preparation of tert-butyl 4-(3-ethoxy-3-oxo-propyl)-4-fluoro-piperidine-1-carboxylate

To a solution of tert-butyl 4-[(E)-3-ethoxy-3-oxo-prop-1-enyl]-4-fluoro-piperidine-1-carboxylate (0.65 g, 2.16 mmol, 1.0 equiv.) in EtOH (20 mL) was added Pd/C (459.08 mg, 431.39 μmol, 10% purity, 0.2 equiv.). The mixture was stirred at 25° C. for 12 hours under a N₂ atmosphere. The mixture was filtered by EtOH (100 mL), and the filtrate was concentrated under reduced pressure to give tert-butyl 4-(3-ethoxy-3-oxo-propyl)-4-fluoro-piperidine-1-carboxylate (1.25 g, crude) as a yellow oil.

¹H NMR (400 MHz, DMSO-d₆) δ=4.10-4.01 (m, 2H), 3.90 (d, J=8.8 Hz, 1H), 3.76 (d, J=10.4 Hz, 1H), 3.07-2.80 (m, 2H), 2.41-2.37 (m, 1H), 2.35-2.27 (m, 1H), 2.01-1.80 (m, 2H), 1.80-1.66 (m, 2H), 1.65-1.50 (m, 2H), 1.41-1.38 (m, 9H), 1.21-1.15 (m, 3H)

### Step D. Procedure for Preparation of tert-butyl 4-fluoro-4-(3-hydroxypropyl)piperidine-1-carboxylate

To a solution of tert-butyl 4-(3-ethoxy-3-oxo-propyl)-4-fluoro-piperidine-1-carboxylate (0.6 g, 1.98 mmol, 1.0 equiv.) in THE (50 mL) was added LiAlH₄ (2.5 M, 949.34 μL, 1.2 equiv.) under a N₂ atmosphere. The mixture was stirred at 0° C. for 1 hour. The reaction mixture was quenched by addition of Na₂SO₄ 10H₂O (1 g) under 0° C., filtered, and concentrated under reduced pressure to give a residue. The residue was purified by flash silica gel chromatography (0˜45% ethyl acetate/petroleum ether) to give tert-butyl 4-fluoro-4-(3-hydroxypropyl)piperidine-1-carboxylate (0.92 g, 3.52 mmol, 89.00% yield) as a yellow oil.

¹H NMR (400 MHz, DMSO-d₆) δ=4.44 (t, J=4.8 Hz, 1H), 4.00-3.66 (m, 2H), 3.42-3.34 (m, 2H), 3.09-2.78 (m, 2H), 1.72 (t, J=11.2 Hz, 1H), 1.65-1.54 (m, 3H), 1.52-1.45 (m, 2H), 1.43-1.37 (m, 10H), 1.26-1.14 (m, 1H)

### Step E. Procedure for Preparation of tert-butyl 4-[3-(4-bromo-3-methyl-phenoxy)propyl]-4-fluoro-piperidine-1-carboxylate

A mixture of tert-butyl 4-fluoro-4-(3-hydroxypropyl)piperidine-1-carboxylate (460 mg, 1.76 mmol, 1.0 equiv.), 4-bromo-3-methyl-phenol (362.14 mg, 1.94 mmol, 1.1 equiv.), and 2-(tributyl-phosphanylidene)acetonitrile (637.25 mg, 2.64 mmol, 1.5 equiv.) in toluene (20 mL) was degassed and purged with N₂ three times. The mixture was stirred at 125° C. for 12 hours under N₂ atmosphere. The reaction mixture was concentrated under reduced pressure to give a residue. The residue was purified by flash silica gel chromatography (0-1% ethyl acetate/petroleum ether) to give tert-butyl 4-[3-(4-bromo-3-methyl-phenoxy)propyl]-4-fluoro-piperidine-1-carboxylate (700 mg, 1.63 mmol, 92.41% yield) as a yellow oil.

### Step F. Procedure for Preparation of 4-[3-(4-bromo-3-methyl-phenoxy)propyl]-4-fluoro-piperidine

A solution of tert-butyl 4-[3-(4-bromo-3-methyl-phenoxy)propyl]-4-fluoro-piperidine-1-carboxylate (700 mg, 1.63 mmol, 1.0 equiv.) in HCl/dioxane (5 mL) and dioxane (3 mL) was stirred at 25° C. for 12 hours. The reaction mixture was concentrated under reduced pressure to give 4-[3-(4-bromo-3-methyl-phenoxy)propyl]-4-fluoro-piperidine (530 mg, crude) as a white solid. MS (ESI) m/z: 332.0 [M+H]⁺.

### Step G. Procedure for Preparation of ethyl 2-[4-[3-(4-bromo-3-methyl-phenoxy)propyl]-4-fluoro-1-piperidyl]acetate

To a solution of 4-[3-(4-bromo-3-methyl-phenoxy)propyl]-4-fluoro-piperidine (530 mg, 1.60 mmol, 1.0 equiv.) and ethyl 2-bromoacetate (536.05 mg, 3.21 mmol, 355.23 μL, 2.0 equiv.) in DMF (5 mL) was added K₂CO₃ (665.43 mg, 4.81 mmol, 3.0 equiv.). The mixture was stirred at 40° C. for 5.5 hours. The reaction mixture was concentrated under reduced pressure to give a residue. The crude product was purified by reversed-phase HPLC to give ethyl 2-[4-[3-(4-bromo-3-methyl-phenoxy)propyl]-4-fluoro-1-piperidyl]acetate (160 mg, 372.79 μmol, 23.23% yield) as a yellow oil. MS (ESI) m/z: 416.1 [M+H]⁺.

### Step H. Procedure for Preparation of tert-butyl 6-[8-(1,3-benzothiazol-2-ylcarbamoyl)-3,4-dihydro-1H-isoquinolin-2-yl]-3-[4-[3-[1-(2-ethoxy-2-oxo-ethyl)-4-fluoro-4-piperidyl]propoxy]-2-methyl-phenyl]pyridine-2-carboxylate

A mixture of ethyl 2-[4-[3-(4-bromo-3-methyl-phenoxy)propyl]-4-fluoro-1-piperidyl]acetate (160 mg, 384.32 μmol, 1.0 equiv.), tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (258.95 mg, 422.75 μmol, 1.1 equiv.), KF (66.98 mg, 1.15 mmol, 27.01 μL, 3.0 equiv.), and Ad₂nBuP Pd G₃ (55.98 mg, 76.86 μmol, 0.2 equiv.) in dioxane (5 mL) and H₂O (0.5 mL) was degassed and purged with N₂ three times. The mixture was stirred at 100° C. for 1 hour under N₂ atmosphere. The reaction mixture was partitioned between ethyl acetate (30 mL) and water (20 mL). The organic phase was separated, washed with brine (20 mL), dried over Na₂SO₄, filtered, and concentrated under reduced pressure to give a residue. The residue was purified by prep-TLC (SiO₂, petroleum ether: ethyl acetate=0:1) to give tert-butyl 6-[8-(1,3-benzothiazol-2-ylcarbamoyl)-3,4-dihydro-1H-isoquinolin-2-yl]-3-[4-[3-[1-(2-ethoxy-2-oxo-ethyl)-4-fluoro-4-piperidyl]propoxy]-2-methyl-phenyl]pyridine-2-carboxylate (240 mg, 268.61 μmol, 69.89% yield) as a yellow solid.

MS (ESI) m/z: 822.4 [M+H]⁺.

### Step I. Procedure for preparation of 2-[4-[3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]propyl]-4-fluoro-1-piperidyl]acetic acid

To a solution of tert-butyl 6-[8-(1,3-benzothiazol-2-ylcarbamoyl)-3,4-dihydro-1H-isoquinolin-2-yl]-3-[4-[3-[1-(2-ethoxy-2-oxo-ethyl)-4-fluoro-4-piperidyl]propoxy]-2-methyl-phenyl]pyridine-2-carboxylate (240 mg, 291.97 μmol, 1.0 equiv.) in THE (2.4 mL) and H₂O (0.8 mL) was added LiOH·H₂O (36.76 mg, 875.92 μmol, 3.0 equiv.). The mixture was stirred at 25° C. for 2 hours. The reaction mixture was acidified to pH=3-4 with citric acid and partitioned between DCM (30 mL) and water (15 mL). The organic phase was separated, washed with brine (15 mL), dried over Na₂SO₄, filtered, and concentrated under reduced pressure to give 2-[4-[3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]propyl]-4-fluoro-1-piperidyl]acetic acid (230 mg, crude) as a white solid. MS (ESI) m/z: 794.7 [M+H]⁺.

### Step J. Procedure for Preparation of tert-butyl 6-[8-(1,3-benzothiazol-2-ylcarbamoyl)-3,4-dihydro-1H-isoquinolin-2-yl]-3-[4-[3-[1-[2-[[3-(2,6-dioxo-3-piperidyl)-1-methyl-indazol-6-yl]amino]-2-oxo-ethyl]-4-fluoro-4-piperidyl]propoxy]-2-methyl-phenyl]pyridine-2-carboxylate

To a solution of 2-[4-[3-[4-[6-[8-(1,3-benzothiazol-2-ylcarbamoyl)-3,4-dihydro-1H-isoquinolin-2-yl]-2-tert-butoxycarbonyl-3-pyridyl]-3-methyl-phenoxy]propyl]-4-fluoro-1-piperidyl]acetic acid (230 mg, 289.69 μmol, 1.0 equiv.) and 3-(6-amino-1-methyl-indazol-3-yl)piperidine-2,6-dione (149.64 mg, 579.39 μmol, 2.0 equiv.) in pyridine (2 mL) was added EDCI (111.07 mg, 579.39 μmol, 2.0 equiv.). The mixture was stirred at 40° C. for 2 hours. The reaction mixture was concentrated under reduced pressure to give a residue. The residue was purified by prep-TLC (SiO₂, DCM:MeOH=20:1) to give tert-butyl 6-[8-(1,3-benzothiazol-2-ylcarbamoyl)-3,4-dihydro-1H-isoquinolin-2-yl]-3-[4-[3-[1-[2-[[3-(2,6-dioxo-3-piperidyl)-1-methyl-indazol-6-yl]amino]-2-oxo-ethyl]-4-fluoro-4-piperidyl]propoxy]-2-methyl-phenyl]pyridine-2-carboxylate (230 mg, 206.83 mol, 71.39% yield) as a yellow solid. MS (ESI) m/z: 1034.4 [M+H]⁺.

### Step K. Procedure for Preparation of 6-[8-(1,3-benzothiazol-2-ylcarbamoyl)-3,4-dihydro-1H-isoquinolin-2-yl]-3-[4-[3-[1-[2-[[3-(2,6-dioxo-3-piperidyl)-1-methyl-indazol-6-yl]amino]-2-oxo-ethyl]-4-fluoro-4-piperidyl]propoxy]-2-methyl-phenyl]pyridine-2-carboxylic acid

A solution of tert-butyl 6-[8-(1,3-benzothiazol-2-ylcarbamoyl)-3,4-dihydro-1H-isoquinolin-2-yl]-3-[4-[3-[1-[2-[[3-(2,6-dioxo-3-piperidyl)-1-methyl-indazol-6-yl]amino]-2-oxo-ethyl]-4-fluoro-4-piperidyl]propoxy]-2-methyl-phenyl]pyridine-2-carboxylate (230 mg, 222.39 μmol, 1.0 equiv.) in TFA (2 mL) and DCM (2 mL) was stirred at 25° C. for 12 hours. The reaction mixture was concentrated under reduced pressure to give a residue. The crude product was purified by reverse-phase HPLC to give 6-[8-(1,3-benzothiazol-2-ylcarbamoyl)-3,4-dihydro-1H-isoquinolin-2-yl]-3-[4-[3-[1-[2-[[3-(2,6-dioxo-3-piperidyl)-1-methyl-indazol-6-yl]amino]-2-oxo-ethyl]-4-fluoro-4-piperidyl]propoxy]-2-methyl-phenyl]pyridine-2-carboxylic acid (114.06 mg, 108.34 μmol, 48.72% yield) as a yellow solid. MS (ESI) m/z: 978.6 [M+H]⁺. ¹H NMR (400 MHz, DMSO-d₆) δ=13.14-12.29 (m, 2H), 10.97-10.70 (m, 1H), 9.96-9.66 (m, 1H), 8.06-8.00 (m, 2H), 7.78 (d, J=8.0 Hz, 1H), 7.66-7.59 (m, 2H), 7.49-7.41 (m, 3H), 7.39-7.33 (m, 2H), 7.22 (d, J=9.2 Hz, 1H), 6.91 (s, 2H), 6.79 (d, J=2.4 Hz, 1H), 6.71 (dd, J=2.4, 8.4 Hz, 1H), 4.96 (s, 2H), 4.36-4.27 (m, 1H), 4.00-3.94 (m, 2H), 3.93-3.88 (m, 5H), 3.22-3.17 (m, 2H), 3.02 (t, J=5.6 Hz, 2H), 2.77-2.58 (m, 4H), 2.46-2.42 (m, 2H), 2.37-2.28 (m, 2H), 2.17 (dd, J=5.2, 13.2 Hz, 1H), 2.02 (s, 3H), 1.80 (s, 5H), 1.76-1.70 (m, 2H)

### Example 315. Preparation of Compound 357

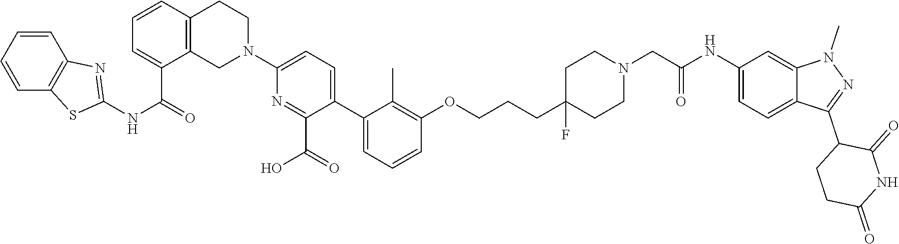 [SMILES: Cc1c(OCCCC2(F)CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]

### 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-2-oxo-ethyl]-4-fluoro-4-piperidyl]propoxy]-2-methyl-phenyl]pyridine-2-carboxylic acid

### Step A. Procedure for Preparation of tert-butyl 4-[3-(3-bromo-2-methyl-phenoxy)propyl]-4-fluoro-piperidine-1-carboxylate

A mixture of tert-butyl 4-fluoro-4-(3-hydroxypropyl)piperidine-1-carboxylate (460 mg, 1.76 mmol, 1.0 equiv.), 3-bromo-2-methyl-phenol (362.14 mg, 1.94 mmol, 1.1 equiv.), and 2-(tributyl-phosphanylidene)acetonitrile (637.25 mg, 2.64 mmol, 1.5 equiv.) in toluene (20 mL) was degassed and purged with N₂ three times. The mixture was stirred at 125° C. for 12 hours under N₂ atmosphere. The reaction mixture was concentrated under reduced pressure to give a residue. The residue was purified by flash silica gel chromatography (0-1% ethyl acetate/petroleum ether) to give tert-butyl 4-[3-(3-bromo-2-methyl-phenoxy)propyl]-4-fluoro-piperidine-1-carboxylate (670 mg, 1.56 mmol, 88.45% yield) as a yellow oil. ¹H NMR (400 MHz, DMSO-d₆) δ=7.16 (d, J=6.8 Hz, 1H), 7.10 (d, J=7.6 Hz, 1H), 6.96 (d, J=8.0 Hz, 1H), 4.11-3.86 (m, 3H), 3.81-3.57 (m, 2H), 2.99 (d, J=3.2 Hz, 1H), 2.31-2.18 (m, 3H), 1.88-1.74 (m, 5H), 1.66-1.51 (m, 2H), 1.41 (s, 9H), 1.11-0.91 (m, 1H)

### Step B. Procedure for Preparation of 4-[3-(3-bromo-2-methyl-phenoxy)propyl]-4-fluoro-piperidine

To a solution of tert-butyl 4-[3-(3-bromo-2-methyl-phenoxy)propyl]-4-fluoro-piperidine-1-carboxylate (670 mg, 1.56 mmol, 1.0 equiv.) in dioxane (3 mL) was added HCl/dioxane (5 mL). The mixture was stirred at 25° C. for 12 hours. The reaction mixture was concentrated under reduced pressure to remove to give 4-[3-(3-bromo-2-methyl-phenoxy)propyl]-4-fluoro-piperidine (530 mg, crude) as a white solid. MS (ESI) m/z: 332.1 [M+H]⁺.

### Step C. Procedure for Preparation of ethyl 2-[4-[3-(3-bromo-2-methyl-phenoxy)propyl]-4-fluoro-1-piperidyl]acetate

To a solution of 4-[3-(3-bromo-2-methyl-phenoxy)propyl]-4-fluoro-piperidine (530 mg, 1.60 mmol, 1.0 equiv.) and ethyl 2-bromoacetate (268.02 mg, 1.60 mmol, 177.62 μL, 1.0 equiv.) in DMF (5 mL) was added K₂CO₃ (665.43 mg, 4.81 mmol, 3.0 equiv.). The mixture was stirred at 40° C. for 2.5 hours. The reaction mixture was concentrated under reduced pressure to give ethyl 2-[4-[3-(3-bromo-2-methyl-phenoxy)propyl]-4-fluoro-1-piperidyl]acetate (620 mg, crude) as a yellow oil. MS (ESI) m/z: 416.2 [M+H]⁺.

### Step D. Procedure for Preparation of tert-butyl 6-[8-(1,3-benzothiazol-2-ylcarbamoyl)-3,4-dihydro-1H-isoquinolin-2-yl]-3-[3-[3-[1-(2-ethoxy-2-oxo-ethyl)-4-fluoro-4-piperidyl]propoxy]-2-methyl-phenyl]pyridine-2-carboxylate

To a solution of ethyl 2-[4-[3-(3-bromo-2-methyl-phenoxy)propyl]-4-fluoro-1-piperidyl]acetate (570 mg, 1.37 mmol, 1.0 equiv.) and tert-butyl 6-[8-(1,3-benzothiazol-2-ylcarbamoyl)-3,4-dihydro-1H-isoquinolin-2-yl]-3-(4,4,5,5-tetramethyl-1,3,2-dioxaborolan-2-yl)pyridine-2-carboxylate (838.65 mg, 1.37 mmol, 1.0 equiv.) in dioxane (3 mL) and H₂O (0.3 mL) was added KF (238.63 mg, 4.11 mmol, 96.22 μL, 3.0 equiv.) and Ad₂nBuP Pd G₃ (199.42 mg, 273.83 μmol, 0.2 equiv.). The mixture was stirred at 100° C. for 1 hour. The reaction mixture was partitioned between ethyl acetate (30 mL) and water (20 mL). The organic phase was separated, washed with brine (20 mL), dried over Na₂SO₄, filtered, and concentrated under reduced pressure to give a residue. The residue was purified by flash silica gel chromatography (0˜45% ethyl acetate/petroleum ether) to give tert-butyl 6-[8-(1,3-benzothiazol-2-ylcarbamoyl)-3,4-dihydro-1H-isoquinolin-2-yl]-3-[3-[3-[1-(2-ethoxy-2-oxo-ethyl)-4-fluoro-4-piperidyl]propoxy]-2-methyl-phenyl]pyridine-2-carboxylate (530 mg, 638.32 μmol, 46.62% yield) as a yellow oil. MS (ESI) m/z: 822.4 [M+H]⁺. 1H NMR (400 MHz, DMSO-d₆) δ=8.02 (d, J=7.6 Hz, 1H), 7.78 (d, J=8.4 Hz, 1H), 7.60 (d, J=7.2 Hz, 1H), 7.45 (d, J=8.4 Hz, 5H), 7.14-7.04 (m, 1H), 6.93 (dd, J=8.4, 14.4 Hz, 2H), 6.58 (d, J=6.8 Hz, 1H), 5.07-4.88 (m, 2H), 4.09-3.86 (m, 6H), 3.25-3.18 (m, 2H), 3.08-2.99 (m, 2H), 2.65-2.60 (m, 2H), 2.41-2.36 (m, 2H), 1.99 (s, 1H), 1.88 (s, 2H), 1.84-1.66 (m, 7H), 1.21-1.14 (m, 4H), 1.00 (s, 9H)

### Step E. Procedure for Preparation of 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-4-fluoro-1-piperidyl]acetic acid

To a solution of tert-butyl 6-[8-(1,3-benzothiazol-2-ylcarbamoyl)-3,4-dihydro-1H-isoquinolin-2-yl]-3-[3-[3-[1-(2-ethoxy-2-oxo-ethyl)-4-fluoro-4-piperidyl]propoxy]-2-methyl-phenyl]pyridine-2-carboxylate (200 mg, 243.31 μmol, 1.0 equiv.) in THE (2.1 mL) and H₂O (0.7 mL) was added LiOH H₂O (30.63 mg, 729.93 μmol, 3.0 equiv.). The mixture was stirred at 25° C. for 2 hours. The reaction mixture was acidified to pH=5-6 with citric acid and partitioned between DCM (20 mL) and water (15 mL). The organic phase was separated, washed with brine (15 mL), dried over Na₂SO₄, filtered, and concentrated under reduced pressure to give 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-4-fluoro-1-piperidyl]acetic acid (120 mg, 151.14 μmol, 62.12% yield) as a white solid. MS (ESI) m/z: 794.4 [M+H]⁺.

### Step F. Procedure for Preparation of tert-butyl 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-2-oxo-ethyl]-4-fluoro-4-piperidyl]propoxy]-2-methyl-phenyl]pyridine-2-carboxylate

To a solution of 2-[4-[3-[3-[6-[8-(1,3-benzothiazol-2-ylcarbamoyl)-3,4-dihydro-1H-isoquinolin-2-yl]-2-tert-butoxycarbonyl-3-pyridyl]-2-methyl-phenoxy]propyl]-4-fluoro-1-piperidyl]acetic acid (120 mg, 151.14 μmol, 1.0 equiv.) and 3-(6-amino-1-methyl-indazol-3-yl)piperidine-2,6-dione (58.56 mg, 226.72 μmol, 1.5 equiv.) in pyridine (1 mL) was added EDCI (57.95 mg, 302.29 mol, 2.0 equiv.). The mixture was stirred at 40° C. for 4 hours. The reaction mixture was concentrated under reduced pressure to give a residue. The residue was purified by prep-TLC (SiO₂, DCM:MeOH=20:1) to give tert-butyl 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-2-oxo-ethyl]-4-fluoro-4-piperidyl]propoxy]-2-methyl-phenyl]pyridine-2-carboxylate (110 mg, 104.23 μmol, 68.96% yield) as a yellow solid. MS (ESI) m/z: 1034.7 [M+H]⁺.

### Step G. Procedure for Preparation of 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-2-oxo-ethyl]-4-fluoro-4-piperidyl]propoxy]-2-methyl-phenyl]pyridine-2-carboxylic acid

A solution of tert-butyl 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-2-oxo-ethyl]-4-fluoro-4-piperidyl]propoxy]-2-methyl-phenyl]pyridine-2-carboxylate (90 mg, 87.02 μmol, 1.0 equiv.) in TFA (1 mL) and DCM (1 mL) was stirred at 40° C. for 1.5 hours. The reaction mixture was concentrated under reduced pressure to give a residue. The residue was purified by reverse-phase HPLC to give 6-[8-(1,3-benzothiazol-2-ylcarbamoyl)-3,4-dihydro-1H-isoquinolin-2-yl]-3-[3-[3-[1-[2-[[3-(2,6-dioxo-3-piperidyl)-1-methyl-indazol-6-yl]amino]-2-oxo-ethyl]-4-fluoro-4-piperidyl]propoxy]-2-methyl-phenyl]pyridine-2-carboxylic acid (36.35 mg, 34.56 μmol, 39.71% yield) as a yellow solid. MS (ESI) m/z: 978.3 [M+H]⁺. ¹H NMR (400 MHz, DMSO-d₆) δ=10.87 (s, 1H), 9.86 (s, 1H), 8.06-8.00 (m, 2H), 7.78 (d, J=8.0 Hz, 1H), 7.63 (d, J=8.8 Hz, 2H), 7.50-7.42 (m, 3H), 7.40-7.32 (m, 2H), 7.22 (dd, J=1.2, 8.8 Hz, 1H), 7.10 (s, 1H), 6.96 (d, J=9.2 Hz, 1H), 6.88 (d, J=8.0 Hz, 1H), 6.68-6.58 (m, 1H), 4.98 (s, 2H), 4.32 (dd, J=5.2, 9.6 Hz, 1H), 4.03-3.95 (m, 2H), 3.94-3.88 (m, 5H), 3.18 (s, 2H), 3.03 (t, J=5.6 Hz, 2H), 2.77-2.69 (m, 2H), 2.68-2.60 (m, 2H), 2.45 (d, J=3.6 Hz, 2H), 2.36-2.31 (m, 1H), 2.21-2.13 (m, 1H), 1.91 (s, 3H), 1.88-1.72 (m, 8H).

### Example 316. Preparation of Compound 271b

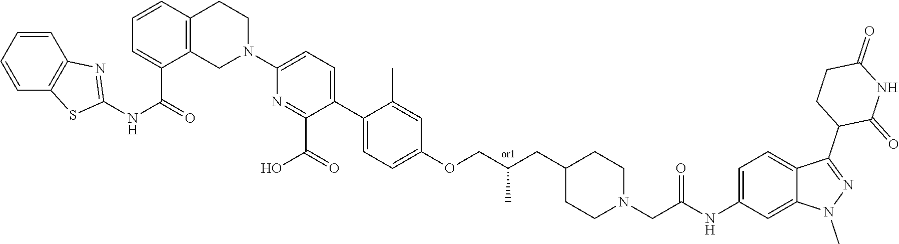 [SMILES: Cc1cc(OC[C@@H](C)CC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O]

### 6-(8-(benzo[d]thiazol-2-ylcarbamoyl)-3,4-dihydroisoquinolin-2(1-H)-yl)-3-(4-(2S-((3-(1-(2-((3-(2,6-dioxopiperidin-3-yl)-1-methyl-1H-indazol-6-yl)amino)-2-oxoethyl)piperidin-4-yl)-2-methylpropoxy)-2-methylphenyl)picolinic acid

### [rel-6-(8-(benzo[d]thiazol-2-ylcarbamoyl)-3,4-dihydroisoquinolin-2(1H)-yl)-3-(4-((2S)-3-(1-(2-((3-(2,6-dioxopiperidin-3-yl)-1-methyl-1H-indazol-6-yl)amino)-2-oxoethyl)piperidin-4-yl)-2-methylpropoxy)-2-methylphenyl)picolinic acid]

### Step A. Procedure for Preparation of (S)-4-(3-(4-bromo-3-methylphenoxy)-2-methylpropyl)piperidine

To a solution of tert-butyl (S)-4-(3-(4-bromo-3-methylphenoxy)-2-methylpropyl)piperidine-1-carboxylate (1 g, 2.35 mmol, 1 equiv.) was added HCl/EtOAc (4 M, 586.32 μL, 1 equiv.). The mixture was stirred at 25° C. for 2 hours. The reaction mixture was filtered and concentrated under reduced pressure to give (S)-4-(3-(4-bromo-3-methylphenoxy)-2-methylpropyl)piperidine (800 mg, crude) as a white solid. MS (ESI) m/z: 326.0 [M+H]⁺.

### Step B. Procedure for Preparation of ethyl (S)-2-(4-(3-(4-bromo-3-methylphenoxy)-2-methylpropyl)piperidin-1-yl)acetate

To a solution of (S)-4-(3-(4-bromo-3-methylphenoxy)-2-methylpropyl)piperidine (300 mg, 919.48 μmol, 1 equiv.), ethyl 2-bromoacetate (153.55 mg, 919.48 μmol, 101.69 μL, 1 equiv.) in MeCN (4 mL) was added K₂CO₃ (381.23 mg, 2.76 mmol, 3 equiv.). The mixture was stirred at 40° C. for 1 hour. The reaction mixture was filtered and concentrated under reduced pressure to give ethyl (S)-2-(4-(3-(4-bromo-3-methylphenoxy)-2-methylpropyl)piperidin-1-yl)acetate (300 mg, crude) as a white solid. MS (ESI) m/z: 412.0 [M+H]⁺.

### Step C. Procedure for Preparation of tert-butyl (S)-6-(8-(benzo[d]thiazol-2-ylcarbamoyl)-3,4-dihydroisoquinolin-2(1H)-yl)-3-(4-(3-(1-(2-ethoxy-2-oxoethyl)piperidin-4-yl)-2-methylpropoxy)-2-methylphenyl)picolinate

A mixture of tert-butyl 6-(8-(benzo[d]thiazol-2-ylcarbamoyl)-3,4-dihydroisoquinolin-2(1H)-yl)-3-(4,4,5,5-tetramethyl-1,3,2-dioxaborolan-2-yl)picolinate (222.82 mg, 363.76 μmol, 1 equiv.), ethyl (S)-2-(4-(3-(4-bromo-3-methylphenoxy)-2-methylpropyl)piperidin-1-yl)acetate (150 mg, 363.76 μmol, 1 equiv.) in dioxane (3 mL) was added KF (1.5 M, 727.52 μL, 3 equiv.) and Ad₂nBuP Pd G₃ (cataCXium® A Pd G₃) (26.49 mg, 36.38 μmol, 0.1 equiv.). The reaction mixture was degassed and purged with N₂ three times. The mixture was stirred at 100° C. for 2 hours under N₂ atmosphere. The reaction mixture was filtered and concentrated under reduced pressure to give a residue. The residue was purified by flash silica gel chromatography (0˜38% ethyl acetate/petroleum ether) to give tert-butyl (S)-6-(8-(benzo[d]thiazol-2-ylcarbamoyl)-3,4-dihydroisoquinolin-2(1H)-yl)-3-(4-(3-(1-(2-ethoxy-2-oxoethyl)piperidin-4-yl)-2-methylpropoxy)-2-methylphenyl)picolinate (180 mg, 184.53 μmol, 50.73% yield) as a yellow solid. MS (ESI) m/z: 818.4 [M+H]⁺. ¹H NMR (400 MHz, CDCl₃) δ=7.84 (d, J=8.0 Hz, 1H), 7.65-7.48 (m, 2H), 7.42-7.27 (m, 5H), 7.02-6.83 (m, 2H), 6.77 (d, J=2.0 Hz, 1H), 6.69 (dd, J=2.8, 5.6 Hz, 1H), 5.04 (s, 2H), 4.21 (q, J=7.2 Hz, 2H), 4.09 (t, J=6.0 Hz, 2H), 3.87-3.66 (m, 2H), 3.36 (s, 2H), 3.16-2.98 (m, 4H), 2.17-1.99 (m, 5H), 1.54-1.43 (m, 4H), 1.33-1.22 (m, 5H), 1.19 (s, 9H), 1.11 (s, 2H), 1.02 (d, J=6.4 Hz, 3H)

### Step D. Procedure for Preparation of (S)-2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)-2-methylpropyl)piperidin-1-yl)acetic acid

To a solution of tert-butyl (S)-6-(8-(benzo[d]thiazol-2-ylcarbamoyl)-3,4-dihydroisoquinolin-2(1H)-yl)-3-(4-(3-(1-(2-ethoxy-2-oxoethyl)piperidin-4-yl)-2-methylpropoxy)-2-methylphenyl)picolinate (180 mg, 220.04 μmol, 1 equiv.) in THE (2 mL) was added LiOH·H₂O (27.70 mg, 660.12 μmol, 3 equiv.) and H₂O (0.5 mL). The mixture was stirred at 25° C. for 1.5 hours. The reaction mixture was concentrated under reduced pressure to remove THF. The aqueous phase was adjusted to pH=4-5 with 1M HCl. The reaction mixture was filtered. The filter cake was diluted in ethyl acetate. The combined organic layers were dried over by Na₂SO₄, filtered and concentrated under reduced pressure to give (S)-2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)-2-methylpropyl)piperidin-1-yl)acetic acid (170 mg, crude) as a yellow solid. MS (ESI) m/z: 790.7 [M+H]⁺.

### Step E. Procedure for Preparation of tert-butyl 6-(8-(benzo[d]thiazol-2-ylcarbamoyl)-3,4-dihydroisoquinolin-2(1H)-yl)-3-(4-((2S)-3-(1-(2-((3-(2,6-dioxopiperidin-3-yl)-1-methyl-1H-indazol-6-yl)amino)-2-oxoethyl)piperidin-4-yl)-2-methylpropoxy)-2-methylphenyl)picolinate

To a solution of (S)-2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)-2-methylpropyl)piperidin-1-yl)acetic acid (160 mg, 202.54 μmol, 1 equiv.) and 3-(6-amino-1-methyl-1H-indazol-3-yl)piperidine-2,6-dione (52.31 mg, 202.54 μmol, 1 equiv.) in pyridine (2 mL) was added EDCI (58.24 mg, 303.81 μmol, 1.5 equiv.). The mixture was stirred at 25° C. for 1 hour. The reaction mixture was quenched by the addition of H₂O (2 mL). The reaction mixture was filtered, and the filter cake was washed with water (5 mL). The filter cake was then diluted in DCM (10 mL). The combined organic layers were dried over Na₂SO₄, filtered, and concentrated under reduced pressure to give tert-butyl 6-(8-(benzo[d]thiazol-2-ylcarbamoyl)-3,4-dihydroisoquinolin-2(1H)-yl)-3-(4-((2S)-3-(1-(2-((3-(2,6-dioxopiperidin-3-yl)-1-methyl-1H-indazol-6-yl)amino)-2-oxoethyl)piperidin-4-yl)-2-methylpropoxy)-2-methylphenyl)picolinate (160 mg, crude) as a yellow solid. MS (ESI) m/z: 1030.4 [M+H]⁺.

### Step F. Procedure for Preparation of 6-(8-(benzo[d]thiazol-2-ylcarbamoyl)-3,4-dihydroisoquinolin-2(1H)-yl)-3-(4-((2S)-3-(1-(2-((3-(2,6-dioxopiperidin-3-yl)-1-methyl-1H-indazol-6-yl)amino)-2-oxoethyl)piperidin-4-yl)-2-methylpropoxy)-2-methylphenyl)picolinic acid [rel-6-(8-(benzo[d]thiazol-2-ylcarbamoyl)-3,4-dihydroisoquinolin-2(1H)-yl)-3-(4-((2S)-3-(1-(2-((3-(2,6-dioxopiperidin-3-yl)-1-methyl-1H-indazol-6-yl)amino)-2-oxoethyl)piperidin-4-yl)-2-methylpropoxy)-2-methylphenyl)picolinic acid]

To a solution of tert-butyl 6-(8-(benzo[d]thiazol-2-ylcarbamoyl)-3,4-dihydroisoquinolin-2(1H)-yl)-3-(4-((2S)-3-(1-(2-((3-(2,6-dioxopiperidin-3-yl)-1-methyl-1H-indazol-6-yl)amino)-2-oxoethyl)piperidin-4-yl)-2-methylpropoxy)-2-methylphenyl)picolinate (160 mg, 155.30 μmol, 1 equiv.) in DCM (1 mL) was added TFA (1.54 g, 13.51 mmol, 1 mL, 86.97 equiv.). The mixture was stirred at 25° C. for 1 hour. The reaction mixture was filtered and concentrated under reduced pressure to give a residue which was purified by prep-HPLC to give 6-(8-(benzo[d]thiazol-2-ylcarbamoyl)-3,4-dihydroisoquinolin-2(1H)-yl)-3-(4-((2S)-3-(1-(2-((3-(2,6-dioxopiperidin-3-yl)-1-methyl-1H-indazol-6-yl)amino)-2-oxoethyl)piperidin-4-yl)-2-methylpropoxy)-2-methylphenyl)picolinic acid [rel-6-(8-(benzo[d]thiazol-2-ylcarbamoyl)-3,4-dihydroisoquinolin-2(1H)-yl)-3-(4-((2S)-3-(1-(2-((3-(2,6-dioxopiperidin-3-yl)-1-methyl-1H-indazol-6-yl)amino)-2-oxoethyl)piperidin-4-yl)-2-methylpropoxy)-2-methylphenyl)picolinic acid] (61.56 mg, 59.84 μmol, 58.29% yield) as a yellow solid. MS (ESI) m/z: 974.3 [M+H]⁺. ¹H NMR (400 MHz, DMSO-d₆) δ=13.28-12.39 (m, 1H), 10.89 (s, 1H), 10.66-10.20 (m, 1H), 8.14 (s, 1H), 8.07-7.98 (m, 2H), 7.79 (d, J=8.0 Hz, 1H), 7.72-7.58 (m, 2H), 7.51-7.41 (m, 3H), 7.40-7.32 (m, 2H), 7.17 (d, J=8.8 Hz, 1H), 6.94 (dd, J=8.8, 4.0 Hz, 2H), 6.80 (s, 1H), 6.75-6.67 (m, 1H), 4.97 (s, 2H), 4.34 (dd, J=4.8, 4.8 Hz, 1H), 3.97-3.88 (m, 5H), 3.87-3.66 (m, 4H), 3.02 (t, J=5.2 Hz, 2H), 2.91-2.51 (m, 5H), 2.41-2.28 (m, 1H), 2.19-2.15 (m, 1H), 2.08-1.93 (m, 4H), 1.81 (d, J=11.2 Hz, 2H), 1.66-1.52 (m, 1H), 1.51-1.08 (m, 5H), 1.05-0.93 (m, 3H).

### Example 317. Preparation of Compound 356

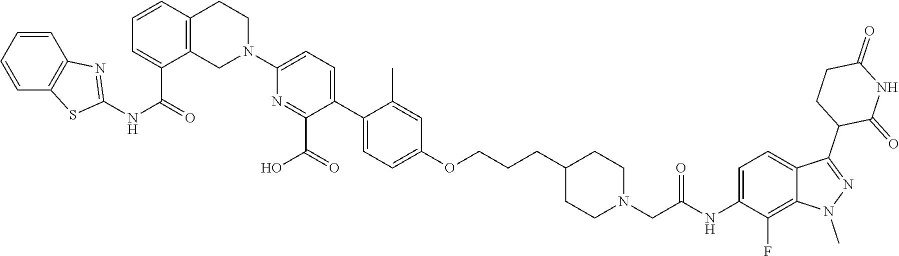 [SMILES: Cc1cc(OCCCC2CCN(CC(=O)Nc3ccc4c(C5CCC(=O)NC5=O)nn(C)c4c3F)CC2)ccc1-c1ccc(N2CCc3cccc(C(=O)Nc4nc5ccccc5s4)c3C2)nc1C(=O)O]

6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-7-fluoro-1-methyl-1H-indazol-6-yl)amino)-2-oxoethyl)piperidin-4-yl)propoxy)-2-methylphenyl)picolinic acid

### Step A. Procedure for Preparation of tert-butyl 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-7-fluoro-1-methyl-1H-indazol-6-yl)amino)-2-oxoethyl)piperidin-4-yl)propoxy)-2-methylphenyl)picolinate

2-(4-(3-(4-(6-(8-(benzo[d]thiazol-2-ylcarbamoyl)-3,4-dihydroisoquinolin-2(1H)-yl)-2-(tert-butoxycarbonyl)pyridin-3-yl)-3-methylphenoxy)propyl)piperidin-1-yl)acetic acid (94 mg, 1 equiv. 0.12 mmol) was dissolved in DCM (5 mL) and DMF (1 mL). To the mixture was added HATU (51 mg, 1.1 equiv. 0.13 mmol), followed by 3-(6-amino-7-fluoro-1-methyl-1H-indazol-3-yl)piperidine-2,6-dione (40 mg, 1.2 equiv. 0.15 mmol) and DIEA (39 mg, 53 μL, 2.5 equiv. 0.30 mmol). The mixture was stirred at 40° C. for 48 hrs. The mixture was concentrated to dryness to afford tert-butyl 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-7-fluoro-1-methyl-1H-indazol-6-yl)amino)-2-oxoethyl)piperidin-4-yl)propoxy)-2-methylphenyl)picolinate, which was carried forward without further purification. MS (ESI) m/z: 517.8 [M+2H]²⁺.

### Step B. Procedure for Preparation of 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-7-fluoro-1-methyl-1H-indazol-6-yl)amino)-2-oxoethyl)piperidin-4-yl)propoxy)-2-methylphenyl)picolinic acid

tert-Butyl 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-7-fluoro-1-methyl-1H-indazol-6-yl)amino)-2-oxoethyl)piperidin-4-yl)propoxy)-2-methylphenyl)picolinate (0.12 g, 1 equiv. 0.12 mmol) was dissolved in DCM (4 mL). To the mixture was added TFA (1 mL). The mixture was stirred at room temperature overnight. The mixture was concentrated, and purified by RP-HPLC to afford 6-(8-(benzo[d]thiazol-2-ylcarbamoyl)-3,4-dihydroisoquinolin-2(1H)-yl)-3-(4-(3-(1-(2-((3-(2,6-dioxopiperidin-3-yl)-7-fluoro-1-methyl-1H-indazol-6-yl)amino)-2-oxoethyl)piperidin-4-yl)propoxy)-2-methylphenyl)picolinic acid (24 mg, 25 μmol, 20%) as a white solid. MS (ESI) m/z: 978.5 [M+H]⁺. 1H NMR (400 MHz, DMSO) δ 10.84 (s, 1H), 9.65 (s, 1H), 7.96 (d, J=7.9 Hz, 1H), 7.72 (d, J=8.0 Hz, 1H), 7.59-7.48 (m, 2H), 7.45-7.34 (m, 4H), 7.34-7.23 (m, 2H), 6.86 (t, J=8.8 Hz, 2H), 6.72 (d, J=2.6 Hz, 1H), 6.64 (dd, J=8.4, 2.6 Hz, 1H), 4.90 (s, 2H), 4.30 (dd, J=10.3, 5.0 Hz, 1H), 4.04 (s, 2H), 3.86 (dt, J=15.3, 6.1 Hz, 4H), 3.09 (s, 2H), 2.95 (t, J=6.0 Hz, 2H), 2.85 (d, J=11.0 Hz, 2H), 2.69-2.45 (m, 2H), 2.36-2.26 (m, 1H), 2.15-2.06 (m, 3H), 1.96 (s, 3H), 1.66 (d, J=8.8 Hz, 4H), 1.32 (s, 2H), 1.22 (s, 3H).

The following examples were synthesized using methods similar to those described in Examples 1-317, above.

| Ex- | | | |
|---|---|---|---|
| ample | Comp | LCMS | |
| No. | No. | (ESI) | ¹H NMR |
| 318 | 353a | m/z = | ¹H NMR (400 MHZ, DMSO-d6): δ = 13.22- |
| | | 985.7 | 12.21 (m, 2H), 10.87 (s, 1H), 8.02 (d, J = 8.0 |
| | | [M + H]⁺ | Hz, 1H), 7.78 (d, J = 8.0 Hz, 1H), 7.62 (d, J = |
| | | | 8.0 Hz, 1H), 7.49-7.41 (m, 3H), 7.38-7.31 |
| | | | (m, 3H), 7.09-7.03 (m, 1H), 7.01-6.97 (m, |
| | | | 2H), 6.94 (d, J = 8.4 Hz, 1H), 6.88 (d, J = 8.4 |
| | | | Hz, 1H), 6.61 (d, J = 7.6 Hz, 1H), 4.98 (s, 2H), |
| | | | 4.61 (s, 1H), 4.32 (dd, J = 4.8, 9.6 Hz, 1H), |
| | | | 4.22 (s, 3H), 3.91 (t, J = 5.6 Hz, 2H), 3.22 (s, |
| | | | 4H), 3.02 (t, J = 6.0 Hz, 2H), 2.68-2.59 (m, |
| | | | 4H), 2.21-2.10 (m, 2H), 1.96-1.89 (m, 5H), |
| | | | 1.82-1.77 (m, 2H), 1.58-1.50 (m, 4H), 1.37- |
| | | | 1.33 (m, 4H), 1.29-1.23 (m, 6H). |
| 319 | 353b | m/z = | ¹H NMR (400 MHZ, DMSO-d6): δ = 13.04- |
| | | 985.4 | 12.23 (m, 2H), 10.87 (s, 1H), 8.02 (d, J = 7.6 |
| | | [M + H]⁺ | Hz, 1H), 7.81-7.76 (m, 1H), 7.62 (d, J = 7.2 |
| | | | Hz, 1H), 7.49-7.41 (m, 3H), 7.40-7.31 (m, |
| | | | 3H), 7.10-7.04 (m, 1H), 7.00 (d, J = 5.2 Hz, |
| | | | 2H), 6.93 (dd, J = 8.4, 14.0 Hz, 2H), 6.61 (d, |
| | | | J = 7.6 Hz, 1H), 4.97 (s, 2H), 4.32 (dd, J = 5.2, |
| | | | 9.6 Hz, 1H), 4.23 (s, 3H), 4.21-4.16 (m, 1H), |
| | | | 3.94-3.88 (m, 2H), 3.24-3.18 (m, 2H), 3.08- |
| | | | 2.98 (m, 2H), 2.71-2.59 (m, 4H), 2.19-2.14 |
| | | | (m, 1H), 2.10-2.05 (m, 2H), 1.86 (s, 3H), |
| | | | 1.83-1.76 (m, 4H), 1.39-1.29 (m, 8H), 1.23 |
| | | | (s, 4H), 1.12-0.99 (m, 3H). |
| 320 | 186b | m/z = | ¹H NMR (400 MHZ, DMSO-d6): δ = 13.27- |
| | | 974.6 | 12.12 (m, 1H), 10.89 (s, 1H), 9.83 (s, 1H), |
| | | [M + H]⁺ | 8.16 (s, 1H), 8.03 (d, J = 8.0 Hz, 1H), 7.78 (d, |
| | | | J = 8.0 Hz, 1H), 7.62 (d, J = 7.6 Hz, 1H), 7.55 |
| | | | (d, J = 8.0 Hz, 1H), 7.48-7.40 (m, 3H), 7.39- |
| | | | 7.28 (m, 3H), 7.06 (td, J = 7.6, 11.6 Hz, 2H), |
| | | | 6.99-6.91 (m, 1H), 6.90-6.84 (m, 1H), 6.62 |
| | | | (d, J = 7.6 Hz, 1H), 4.97 (s, 2H), 4.36 (dd, J = |
| | | | 5.2, 10.0 Hz, 1H), 4.11 (s, 3H), 3.97-3.89 (m, |
| | | | 4H), 3.16 (s, 2H), 3.02 (t, J = 5.6 Hz, 2H), |
| | | | 2.94-2.84 (m, 2H), 2.72-2.62 (m, 2H), 2.38- |
| | | | 2.29 (m, 1H), 2.20-2.04 (m, 2H), 1.91-1.82 |
| | | | (m, 4H), 1.79-1.66 (m, 4H), 1.62 (t, J = 10.0 |
| | | | Hz, 2H), 1.50-1.43 (m, 2H), 1.28-1.19 (m, |
| | | | 2H), 0.94-0.79 (m, 1H). |
| 321 | 210b | m/z = | ¹H NMR (400 MHZ, DMSO-d6): δ = 13.02- |
| | | 960.4 | 12.70 (m, 1H), 10.90 (s, 1H), 10.19-9.78 (m, |
| | | [M + H]⁺ | 1H), 8.13 (s, 1H), 8.03 (d, J = 8.0 Hz, 1H), |
| | | | 7.79 (d, J = 8.0 Hz, 1H), 7.66-7.55 (m, 2H), |
| | | | 7.50-7.41 (m, 3H), 7.40-7.32 (m, 2H), 7.28 |
| | | | (d, J = 7.6 Hz, 1H), 7.12-7.03 (m, 2H), 6.96 |
| | | | (d, J = 8.8 Hz, 1H), 6.87 (d, J = 8.0 Hz, 1H), |
| | | | 6.62 (d, J = 7.6 Hz, 1H), 4.98 (s, 2H), 4.41- |
| | | | 4.34 (m, 1H), 4.10 (s, 3H), 3.99-3.88 (m, |
| | | | 4H), 3.63-3.50 (m, 1H), 3.03 (t, J = 6.0 Hz, |
| | | | 4H), 2.74-2.52 (m, 4H), 2.47-2.21 (m, 2H), |
| | | | 2.21-2.00 (m, 2H), 1.89 (s, 3H), 1.83-1.72 |
| | | | (m, 4H), 1.71-1.56 (m, 2H), 1.44-1.30 (m, |
| | | | 2H). |
| 322 | 218b | m/z = | ¹H NMR (400 MHZ, DMSO-d6): δ = 13.03- |
| | | 1029.4 | 12.32 (m, 2H), 10.90 (s, 1H), 9.79 (s, 1H), |
| | | [M + H]⁺ | 8.14 (s, 1H), 8.03 (d, J = 8.0 Hz, 1H), 7.79 (d, |
| | | | J = 8.0 Hz, 1H), 7.65-7.57 (m, 2H), 7.50- |
| | | | 7.41 (m, 3H), 7.40-7.32 (m, 2H), 7.25-7.19 |
| | | | (m, 1H), 7.12-7.04 (m, 2H), 6.96 (d, J = 8.8 |
| | | | Hz, 1H), 6.86 (d, J = 8.0 Hz, 1H), 6.63 (d, J = |
| | | | 7.6 Hz, 1H), 4.98 (s, 2H), 4.38 (dd, J = 10.0, |
| | | | 5.2 Hz, 1H), 4.08 (s, 3H), 4.01 (t, J = 5.6 Hz, |
| | | | 2H), 3.91 (t, J = 5.6 Hz, 2H), 3.79-3.67 (m, |
| | | | 2H), 3.51-3.44 (m, 1H), 3.02 (t, J = 5.6 Hz, |
| | | | 3H), 2.87-2.75 (m, 2H), 2.75-2.62 (m, 2H), |
| | | | 2.61-2.53 (m, 3H), 2.48-2.41 (m, 2H), 2.40- |
| | | | 2.31 (m, 1H), 2.21-2.12 (m, 1H), 1.96-1.85 |
| | | | (m, 5H). |
| 323 | 275b | m/z = | ¹H NMR (400 MHZ, DMSO-d6): δ = 12.85 |
| | | 1000.5 | 1(s, H), 10.87 (s, 1H), 8.08-8.00 (m, 1H), |
| | | [M + H]⁺ | 7.84-7.76 (m, 1H), 7.66-7.52 (m, 2H), 7.50- |
| | | | 7.41 (m, 3H), 7.40-7.32 (m, 2H), 7.13-7.05 (m, |
| | | | 1H), 7.04-6.90 (m, 4H), 6.62 (d, J = 7.6 Hz, |
| | | | 1H), 5.04-4.91 (m, 2H), 4.32-4.18 (m, 2H), |
| | | | 3.92 (s, 6H), 3.03 (d, J = 4.8 Hz, 3H), 2.66 (d, |
| | | | J = 4.8 Hz, 1H), 2.63-2.60 (m, 1H), 2.54 (s, |
| | | | 6H), 2.33 (s, 1H), 2.22-2.02 (m, 4H), 1.87 (s, |
| | | | 3H), 1.85-1.73 (m, 3H), 1.38 (d, J = 5.6 Hz, |
| | | | 6H), 1.31-1.20 (m, 3H), 1.14-1.05 (m, 2H). |
| 324 | 311b | m/z = | ¹H NMR (400 MHZ, DMSO-d6): δ = 12.90- |
| | | 493.7 | 12.84 (m, 1H), 12.78-12.40 (m, 1H), 10.86 (s, |
| | | [M/2 + | 1H), 8.03 (d, J = 7.6 Hz, 1H), 7.79 (d, J = 8.0 |
| | | H]⁺ | Hz, 1H), 7.68-7.55 (m, 1H), 7.51-7.41 (m, |
| | | | 4H), 7.40-7.33 (m, 3H), 7.10-7.04 (m, 1H), |
| | | | 6.99-6.79 (m, 3H), 6.63-6.58 (m, 1H), 4.98 |
| | | | (s, 2H), 4.29-4.23 (m, 1H), 4.22-4.13 (m, |
| | | | 1H), 3.95-3.88 (m, 5H), 3.76 (d, J = 11.2 Hz, |
| | | | 2H), 3.02 (t, J = 5.6 Hz, 2H), 2.79-2.71 (m, |
| | | | 1H), 2.64-2.58 (m, 2H), 2.19-2.13 (m, 1H), |
| | | | 2.11-2.03 (m, 2H), 1.87 (s, 3H), 1.78 (d, J = |
| | | | 12.8 Hz, 4H), 1.46-1.20 (m, 16H). |
| 325 | 339b | m/z = | ¹H NMR (400 MHZ, DMSO-d6): δ 12.78 (s, |
| | | 1067.4 | 1H), 12.49 (s, 1H), 10.77 (s, 1H), 7.96 (d, J = |
| | | [M + H]⁺ | 7.9 Hz, 1H), 7.72 (d, J = 8.1 Hz, 1H), 7.55 (d, |
| | | | J = 7.5 Hz, 1H), 7.43 (d, J = 9.2 Hz, 1H), 7.44- |
| | | | 7.33 (m, 3H), 7.29 (dt, J = 9.6, 7.5 Hz, 2H), |
| | | | 7.04-6.80 (m, 5H), 6.54 (d, J = 7.4 Hz, 1H), |
| | | | 4.91 (s, 3H), 4.19 (dd, J = 9.2, 5.1 Hz, 1H), |
| | | | 4.11 (d, J = 10.9 Hz, 1H), 3.83 (d, J = 9.2 Hz, |
| | | | 4H), 3.34 (d, J = 15.8 Hz, 2H), 3.13 (d, J = |
| | | | 12.4 Hz, 1H), 2.96 (t, J = 6.0 Hz, 2H), 2.88 |
| | | | (d, J = 10.8 Hz, 1H), 2.55 (dt, J = 8.3, 5.1 Hz, |
| | | | 2H), 2.26 (dp, J = 20.3, 6.9 Hz, 4H), 2.09 (dd, |
| | | | J = 13.2, 6.0 Hz, 1H), 2.01 (dd, J = 9.2, 4.4 |
| | | | Hz, 3H), 1.85 (d, J = 6.3 Hz, 1H), 1.80 (s, 3H), |
| | | | 1.71 (d, J = 12.5 Hz, 2H), 1.39 (t, J = 7.0 Hz, |
| | | | 1H), 1.28 (d, J = 15.5 Hz, 1H), 1.28 (s, 6H), |
| | | | 1.17 (d, J = 7.1 Hz, 2H), 0.98 (q, J = 11.9 Hz, |
| | | | 2H). |

### Example P1

### Resolution of Stereoisomeric Mixtures

Mixtures of stereoisomers were resolved using methods described in Table P1:

TABLE P1

| | | Retention | |
|---|---|---|---|
| Starting Material | Separation Method | Time | Final Product |
| tert-butyl 3-[(3-bromo-2- | Column: Chiralcel OD-3 (50 × | R_{T} = 1.372 | 262b |
| methyl-phenoxy)methyl]-8- | 4.6 mm, internal diameter: 3 μm); | min | (Example 193) |
| azaspiro[4.5]decane-8- | mobile phase: Phase A (CO₂), and | R_{T} = 1.451 | 262a |
| carboxylate | Phase B (MeOH with 0.05% | min | (Example 194) |
| | diethylamine); gradient elution: B in | | |
| | A from 5% to 40%; flow rate: | | |
| | 3 mL/min; detector: PDA; | | |
| | wavelength: 220 nm; column | | |
| | temperature: 35° C.; back pressure: | | |
| | 100 bar | | |
| ethyl 2-[4-[3-(3-bromo-2- | Column: Chiralcel OJ-3 (50 × | R_{T} = 0.957 | 263a |
| methyl-phenoxy)-2-methyl- | 4.6 mm, internal diameter: 3 μm); | min | (Example 70) |
| propyl]-1-piperidyl]acetate | mobile phase: Phase A (CO₂), and | R_{T} = 1.151 | 263b |
| | Phase B (MeOH with 0.05% | min | (Example 199) |
| | diethylamine); gradient elution: B in | | |
| | A from 5% to 40%; flow rate: | | |
| | 3 mL/min; detector: PDA; | | |
| | wavelength: 220 nm; column | | |
| | temperature: 35° C.; back pressure: | | |
| | 100 bar | | |
| tert-butyl 4-[3-(4-bromo-3- | Column: Chiralpak AD-3 (50 × | R_{T} = 1.431 | 266a |
| methyl-phenoxy)-1-methyl- | 4.6 mm, internal diameter: 3 μm); | min | (Example 215) |
| propyl]piperidine-1- | mobile phase: Phase A (CO₂), and | R_{T} = 1.601 | 266b |
| carboxylate | Phase B (MeOH with 0.05% | min | (Example 181) |
| | diethylamine); gradient elution: | | |
| | MeOH (0.05% diethylamine) in CO₂ | | |
| | from 5% to 40%; flow rate: | | |
| | 3 mL/min; detector: PDA; | | |
| | wavelength: 220 nm; column | | |
| | temperature: 35°C; back pressure: | | |
| | 100 bar | | |
| tert-butyl 3-(3-bromo-2- | Column: Chiralcel OJ-3 (50 × | R_{T} = 1.149 | 268a |
| methyl-phenoxy)-8- | 4.6 mm, internal diameter: 3 μm); | min | (Example 204) |
| azaspiro[4.5]decane-8- | mobile phase: Phase A (CO₂), and | R_{T} = 1.342 | 268b |
| carboxylate | Phase B (MeOH with 0.05% | min | (Example 205) |
| | diethylamine); gradient elution: B in | | |
| | A from 5% to 40%; flow rate: | | |
| | 3 mL/min; detector: PDA; | | |
| | wavelength: 220 nm; column | | |
| | temperature: 35°C; back pressure: | | |
| | 100 bar | | |
| ethyl 2-[3-[2-(3-bromo-2- | Column: Chiralcel OJ-3 (50 × | R_{T} = 1.204 | 272a |
| methyl-phenoxy)ethyl]-8- | 4.6 mm, internal diameter: 3 μm); | min | (Example 72) |
| azaspiro [4.5]decan-8- | mobile phase: Phase A (CO₂), and | R_{T} = 1.374 | 272b |
| yl]acetate | Phase B (EtOH with 0.05% | min | (Example 206) |
| | diethylamine); gradient elution: B in | | |
| | A from 5% to 40%; flow rate: | | |
| | 3 mL/min; detector: PDA; | | |
| | wavelength: 220 nm; column | | |
| | temperature: 35° C.; back pressure: | | |
| | 100 bar | | |
| tert-butyl 4-[3-(4-bromo-3- | Column: Daicel ChiralPak AY-H | R_{T} = 0.831 | 276a |
| methyl- | (250 mm × 30 mm, 5 μm); mobile | min | (Example 77) |
| phenoxy)butyl]piperidine-1- | phase: Phase A (CO₂), and Phase B | R_{T} = 1.029 | 276b |
| carboxylate | (EtOH with 0.05% diethylamine); | min | (Example 78) |
| | gradient elution: B in A from 5% to | | |
| | 40%; flow rate: 3 mL/min; detector: | | |
| | PDA; wavelength: 220 nm; column | | |
| | temperature: 35° C.; back pressure: | | |
| | 100 bar | | |
| tert-butyl 4-(3-(4-bromo-3- | Column: Chiralpak IC-3 (50 × | R_{T} = 1.794 | 271a |
| methylphenoxy)-2- | 4.6 mm, internal diameter: 3 μm); | min | (Example 183) |
| methylpropyl)piperidine-1- | mobile phase: Phase A (CO₂), and | R_{T} = 1.434 | 271b |
| carboxylate | Phase B (iPrOH with 0.05% | min | (Example 316) |
| | diethylamine); gradient elution: B in | | |
| | A from 5% to 40%; flow rate: | | |
| | 3 mL/min; detector: PDA; | | |
| | wavelength: 220 nm; column | | |
| | temperature: 35° C.; back pressure: | | |
| | 100 bar | | |
| 4-((1r,4s)-4-(3-bromo-2- | Column: Chiralpak IG-3 (50 × 3.0 | R_{T} = 1.377 | 281a |
| methylphenoxy)cyclohexyl) | mm, internal diameter: 3 μm); mobile | min | (Example 207) |
| butan-2-ol | phase: Phase A (CO₂), and Phase B | | 283a |
| | (MeOH with 0.05% diethylamine); | | (Example 209) |
| | gradient elution: B in A from 5% to | R_{T} = 1.179 | 281b |
| | 40%; flow rate: 3 mL/min; detector: | min | (Example 208) |
| | PDA; wavelength: 220 nm; column | | 283b |
| | temperature: 30° C.; back pressure: | | (Example 211) |
| | 100 bar | | |
| 3-[4-(3-bromo-2-methyl- | Column: Daicel ChiralPak AD | R_{T} = 1.657 | 282a |
| phenoxy)cyclohexyl]-2- | (250 mm × 30 mm, 10 μm); mobile | min | (Example 184) |
| methyl-propan-1-ol | phase: Phase A (CO₂), and Phase B | | 284a |
| | (MeOH with 0.05% diethylamine); | | (Example 210) |
| | gradient elution: B in A from 5% to | R_{T} = 1.474 | 282b |
| | 40%; flow rate: 3 mL/min; detector: | min | (Example 185) |
| | PDA; wavelength: 220 nm; column | | 284b |
| | temperature: 35° C.; back pressure: | | (Example 113) |
| | 100 bar | | |
| ethyl 2-(1-(2-(3-bromo-2- | Column: Chiralcel OJ-3 (50 × | R_{T} = 1.186 | 289a |
| methylphenoxy)ethyl)-6- | 4.6 mm, internal diameter: 3 μm); | min | (Example 116) |
| azaspiro[2.5]octan-6- | mobile phase: Phase A (CO₂), and | R_{T} = 1.338 | 289b |
| yl)acetate | Phase B (iPrOH with 0.05% | min | (Example 81) |
| | diethylamine); gradient elution: B in | | |
| | A from 5% to 40%; flow rate: | | |
| | 3 mL/min; detector: PDA; | | |
| | wavelength: 220 nm; column | | |
| | temperature: 35° C.; back pressure: | | |
| | 100 bar | | |
| 4-((1r,4r)-4-(3-bromo-2- | Column: Chiralpak AD-3 (50 × | R_{T} = 1.293 | 307a |
| methylphenoxy)cyclohexyl)- | 4.6 mm, internal diameter: 3 μm); | min | (Example 250) |
| 3-methylbutan-1-ol | mobile phase: Phase A (CO₂), and | R_{T} = 1.342 | 307b |
| | Phase B (EtOH with 0.05% | min | (Example 251) |
| | diethylamine); gradient elution: B in | | |
| | A from 5% to 40%; flow rate: | | |
| | 3 mL/min; detector: PDA; | | |
| | wavelength: 220 nm; column | | |
| | temperature: 35° C.; back pressure: | | |
| | 100 bar | | |
| 4-((1r,4s)-4-(3-bromo-2- | Column: Chiralpak AD-3 (50 × | R_{T} = 1.886 | 308a |
| methylphenoxy)cyclohexyl)- | 4.6 mm, internal diameter: 3 μm); | min | (Example 252) |
| 2-methylbutan-1-ol | mobile phase: Phase A (CO₂), and | R_{T} = 1.748 | 308b |
| | Phase B (MeOH with 0.05% | min | (Example 269) |
| | diethylamine); gradient elution: B in | | |
| | A from 5% to 40%; flow rate: | | |
| | 3 mL/min; detector: PDA; | | |
| | wavelength: 220 nm; column | | |
| | temperature: 35° C.; back pressure: | | |
| | 100 bar | | |
| 3-((1r,4r)-4-(3-bromo-2- | Column: Chiralpak IF-3 (50 × | R_{T} = 1.522 | 310a |
| methylphenoxy)cyclohexyl) | 4.6 mm, internal diameter: 3 μm); | min | (Example 243) |
| butan-1-ol | mobile phase: Phase A (Hexanes | R_{T} = 1.695 | 310b |
| | (0.05% iPrOH)), and Phase B (EtOH | min | (Example 244) |
| | with 0.05% iPrOH); gradient elution: | | |
| | B in A from 5% to 40%; flow rate: | | |
| | 3 mL/min; detector: PDA; | | |
| | wavelength: 254 nm; column | | |
| | temperature: 35° C.; back pressure: | | |
| | 100 bar | | |
| tert-butyl 3-[3-(2,6- | Column: Chiralpak AD-3 (50 × | R_{T} = 1.202 | 331a |
| dibenzyloxy-3-pyridyl)-1- | 4.6 mm, internal diameter: 3 μm); | min | (Example 263) |
| methyl-indazol-6- | mobile phase: Phase A (CO₂), and | | 332a |
| yl]pyrrolidine-1-carboxylate | Phase B (EtOH with 0.05% | | (Example 265) |
| | diethylamine); isocratic elution: 40% | R_{T} = 1.539 | 331b |
| | B in A; flow rate: 3 mL/min; detector: | min | (Example 264) |
| | PDA; wavelength: 220 nm; column | | 332b |
| | temperature: 35° C.; back pressure: | | (Example 266) |
| | 100 bar | | |
| tert-butyl 3-[3-(2,6- | Column: Chiralpak AD-3 (50 × | R_{T} = 1.195 | 320a |
| dibenzyloxy-3-pyridyl)-1- | 4.6 mm, internal diameter: 3 μm); | min | (Example 247) |
| methyl-indazol-7- | mobile phase: Phase A (CO₂), and | | 321a |
| yl]pyrrolidine-1-carboxylate | Phase B (iPrOH with 0.05% | | (Example 245) |
| | diethylamine); isocratic elution: 40% | R_{T} = 1.556 | 320b |
| | B in A; flow rate: 3 mL/min; detector: | min | (Example 248) |
| | PDA; wavelength: 220 nm; column | | 321b |
| | temperature: 35°C; back pressure: | | (Example 246) |
| | 100 bar | | |
| tert-butyl 4-(5-((1r,4s)-4-(3- | Column: Daicel ChiralPak IG | R_{T} = 0.752 | 333a |
| bromo-2- | (250 mm × 50 mm,10 μm); mobile | min | (Example 267) |
| methylphenoxy)cyclohexyl) | phase: Phase A (CO₂), and Phase B | R_{T} = 1.201 | |
| pentan-2-yl)piperazine-1- | (CO₂—MeOH/MeCN); isocratic | min | |
| carboxylate | elution B%: 40%; wavelength: 220 | | |
| | nm; flow rate: 3 mL/min; detector: | | |
| | PDA; wavelength: 220 nm; column | | |
| | temperature: 35° C.; back pressure: | | |
| | 100 bar | | |
| 3-(2,6- | Column: Daicel ChiralPak IG | R_{T} = 1.827 | 333b |
| bis(benzyloxy)pyridin-3-yl)- | (250 mm × 50 mm,10 μm);mobile | min | (Example 313) |
| 6-(4-(5-((1r,4s)-4-(3-bromo- | phase: Phase A (CO₂), and Phase B | R_{T} = 2.066 | |
| 2- | (CO₂—ACN/iPrOH with 0.1% | min | |
| methylphenoxy)cyclohexyl) | NH₃•H₂O); isocratic elution B%: | | |
| pentan-2-yl)piperazin-1-yl)- | 60%; wavelength: 220 nm; flow rate: | | |
| 1-methyl-1H-indazole | 3 mL/min; detector: PDA; | | |
| | wavelength: 220 nm; column | | |
| | temperature: 35° C.; back pressure: | | |
| | 100 bar | | |
| 3-(2,6- | Column: Chiralpak IG-3 (50 × | R_{T} = 1.220 | 349a |
| bis(benzyloxy)pyridin-3-yl)- | 4.6 mm, internal diameter: 3 μm); | min | (Example 298) |
| 7-(4-(5-((1r,4s)-4-(3-bromo- | mobile phase: Phase A (CO₂), and | R_{T} = 2.101 | 349b |
| 2-methylphenoxy) | Phase B (EtOH + MeCN with 0.05% | min | (Example 299) |
| cyclohexyl)pentan-2- | diethylamine); isocratic elution: 60% | | |
| yl)piperazin-1-yl)-1-methyl- | B in A; flow rate: 3 mL/min; detector: | | |
| 1H-indazole | PDA; wavelength: 220 nm; column | | |
| | temperature: 35° C.; back pressure: | | |
| | 100 bar | | |
| tert-butyl 3-(3-(2,6- | Column: Chiralcel OJ-3 (50 × | R_{T} = 1.669 | 351a |
| bis(benzyloxy)pyridin-3-yl)- | 4.6 mm, internal diameter: 3 μm); | min | (Example 307) |
| 1-methyl-1H-indazol-7- | mobile phase: Phase A (CO₂), and | R_{T} = 2.029 | 351b |
| yl)piperidine-1-carboxylate | Phase B (EtOH with 0.05% | min | (Example 308) |
| | diethylamine); gradient elution: B in | | |
| | A from 5% to 40%; flow rate: | | |
| | 3 mL/min; detector: PDA; | | |
| | wavelength: 220 nm; column | | |
| | temperature: 35° C.; back pressure: | | |
| | 100 bar | | |
| tert-butyl 3-(3-(2,6- | Column: (S,S)Whelk-O1 (50 × | R_{T} = 0.822 | 352a |
| bis(benzyloxy)pyridin-3-yl)- | 4.6 mm, internal diameter: 3 μm); | min | (Example 309) |
| 1-methyl-1H-indazol-6- | mobile phase: Phase A (CO₂), and | R_{T} = 0.953 | 352b |
| yl)piperidine-1-carboxylate | Phase B (iPrOH + MeCN with 0.05% | min | (Example 310) |
| | diethylamine); isocratic elution: 40% | | |
| | B in A; flow rate: 3 mL/min; detector: | | |
| | PDA; wavelength: 220 nm; column | | |
| | temperature: 35° C.; back pressure: | | |
| | 100 bar | | |

### Example B1

### HiBiT Based Degradation Assay

Human BCL-X_{L} protein coding open reading frame fused with N-terminal HiBiT coding sequence was synthesized from Integrated DNA Technologies (IDT). Next, N-HiBiT-BCL-X_{L} sequence was cloned into pLV-UBC-PGK-Puro, a lentivirus plasmid purchased from Vectorbuilder, to generate pLV-UBC-N-HiBiT-BCL-X_{L}-PGK-puro. Lentiviral particles were generated from Lenti-X™ 293T cells (Clontech) by co-transfection of pLV-UBC-N-HiBiT-BCL-X_{L}-PGK-puro plasmids and lentiviral packaging plasmid mix (Cellecta). HT-1080 [HT1080](ATCC CCL-121) cells were infected with the lentivirus, and HT1080 cells stably integrated with the lentiviral vectors were established by incubation with 1 μg/mL puromycin (Thermofisher).

HT1080 cells expressing N-terminal HiBiT tagged BCL-X_{L} were dispensed into a 384-well plate pre-spotted with compounds at varying concentrations. Three thousand cells were seeded into each well in 40 μL of RPMI 1640 media. After 6 hours of incubation at 37° C. with 5% CO₂, 30 μL of the NANO-GLO® HiBiT Lytic Detection System working solution (Promega) was added to each well and incubated at room temperature for 30 min. After incubation, luminescence was read on a PHERAstar FSX Plate Reader (BMG). BCL-X_{L} degradation at each indicated concentration was normalized with the DMSO control. The BCL-X_{L} degradation curves were plotted using a four-parameter logistic model. For compounds displaying hook effects, the curve fitting was performed using the bottom point of the hook curves (i.e., the point of Yₘᵢₙ) as the bottom plateau to calculate EC₅₀. Yₘᵢₙ and EC₅₀ values for certain compounds are shown in Table B1.

TABLE B1

| Compound No. | Yₘᵢₙ (24 h) | EC₅₀ (24 h) | Yₘᵢₙ (6 h) | EC₅₀ (6 h) |
|---|---|---|---|---|
| 101 | B | +++ | B | +++ |
| 102 | | | B | +++ |
| 103 | A | +++ | A | +++ |
| 104 | A | +++ | B | +++ |
| 105 | B | ++ | B | +++ |
| 106 | A | +++ | A | +++ |
| 107 | C | + | C | + |
| 108 | | | B | +++ |
| 109 | | | B | +++ |
| 110 | | | B | +++ |
| 111 | | | B | +++ |
| 112 | | | B | +++ |
| 113 | | | B | ++ |
| 114 | | | A | +++ |
| 115 | | | B | +++ |
| 116a | A | +++ | A | +++ |
| 117a | B | +++ | B | +++ |
| 118 | B | + | B | ++ |
| 119 | A | +++ | A | +++ |
| 120 | B | +++ | B | +++ |
| 121 | A | +++ | A | +++ |
| 122 | B | +++ | B | +++ |
| 123 | | | C | ++ |
| 124a | C | +++ | C | + |
| 125a | C | + | C | + |
| 126 | | | B | ++ |
| 127 | A | +++ | B | +++ |
| 128 | C | +++ | C | + |
| 129 | C | +++ | C | + |
| 130 | A | +++ | A | +++ |
| 131a | A | +++ | A | +++ |
| 132 | B | ++ | C | + |
| 133 | A | +++ | A | +++ |
| 134 | B | +++ | B | ++ |
| 135 | | | B | +++ |
| 136 | B | +++ | B | ++ |
| 137 | A | +++ | B | +++ |
| 138a | B | +++ | B | + |
| 138b | B | +++ | | |
| 139 | B | ++ | B | ++ |
| 140 | A | +++ | A | +++ |
| 141 | A | +++ | B | +++ |
| 142 | A | +++ | A | +++ |
| 143 | A | +++ | A | +++ |
| 144 | A | +++ | A | +++ |
| 145 | A | +++ | B | +++ |
| 146a | A | +++ | A | ++ |
| 146b | B | ++ | | |
| 147a | A | +++ | B | +++ |
| 147b | B | +++ | | |
| 148a | A | +++ | B | +++ |
| 148b | A | +++ | | |
| 149 | A | +++ | B | +++ |
| 150 | B | +++ | B | ++ |
| 151 | A | +++ | B | ++ |
| 152 | A | +++ | B | +++ |
| 153 | A | +++ | A | +++ |
| 154a | A | +++ | A | +++ |
| 155 | A | +++ | B | +++ |
| 156 | A | +++ | A | +++ |
| 157 | | | B | +++ |
| 158 | B | ++ | C | +++ |
| 159a | | | B | +++ |
| 160a | A | +++ | | |
| 160b | A | +++ | | |
| 161a | A | +++ | | |
| 162a | A | +++ | B | +++ |
| 162b | B | +++ | C | ++ |
| 163 | A | +++ | | |
| 164a | A | +++ | | |
| 164b | A | +++ | | |
| 165 | B | ++ | | |
| 166 | B | ++ | | |
| 167a | A | +++ | | |
| 167b | B | +++ | | |
| 168a | B | +++ | | |
| 169a | B | +++ | B | ++ |
| 169b | B | +++ | C | +++ |
| 170 | B | ++ | | |
| 171a | B | ++ | | |
| 172 | A | +++ | C | ++ |
| 173a | B | +++ | | |
| 173b | B | ++ | | |
| 174a | A | +++ | | |
| 174b | B | +++ | C | ++ |
| 175 | B | +++ | | |
| 176a | B | +++ | | |
| 177a | B | ++ | | |
| 178 | B | +++ | | |
| 179a | A | +++ | | |
| 180a | B | +++ | | |
| 180b | B | +++ | | |
| 181 | A | +++ | | |
| 182a | B | +++ | | |
| 183a | A | +++ | B | +++ |
| 184 | A | +++ | A | +++ |
| 185a | A | +++ | | |
| 186a | B | +++ | | |
| 186b | C | + | | |
| 187a | B | +++ | B | ++ |
| 187b | A | +++ | B | +++ |
| 188 | A | +++ | | |
| 189 | A | +++ | | |
| 191 | B | +++ | | |
| 192 | A | +++ | | |
| 193a | B | ++ | | |
| 193b | B | ++ | | |
| 194 | B | ++ | B | ++ |
| 195 | B | +++ | C | +++ |
| 196 | B | ++ | | |
| 197a | B | ++ | | |
| 197b | B | ++ | | |
| 198 | A | +++ | | |
| 201 | A | + | B | + |
| 202a | A | +++ | | |
| 202b | A | +++ | | |
| 203 | A | +++ | B | +++ |
| 204a | B | +++ | | |
| 205 | A | +++ | B | +++ |
| 206 | B | +++ | C | + |
| 207a | A | +++ | | |
| 207b | A | +++ | B | +++ |
| 208 | A | +++ | | |
| 209a | B | ++ | | |
| 210a | B | +++ | | |
| 210b | C | +++ | C | + |
| 211a | B | +++ | | |
| 212a | B | +++ | | |
| 213 | A | ++ | C | + |
| 214a | B | +++ | | |
| 215a | C | +++ | | |
| 216a | B | +++ | | |
| 216b | B | +++ | | |
| 217 | B | +++ | B | +++ |
| 218a | B | ++ | C | + |
| 218b | C | ++ | C | + |
| 219a | A | +++ | | |
| 219b | A | +++ | B | +++ |
| 220 | A | +++ | | |
| 221 | A | +++ | | |
| 222 | A | +++ | B | +++ |
| 223 | A | +++ | | |
| 224a | B | +++ | C | + |
| 224b | B | +++ | | |
| 225 | A | +++ | | |
| 226 | B | ++ | C | +++ |
| 227a | A | +++ | | |
| 228 | B | +++ | | |
| 229 | B | +++ | | |
| 230 | A | +++ | | |
| 231a | B | +++ | | |
| 231b | B | +++ | | |
| 232 | A | +++ | | |
| 233 | A | +++ | | |
| 234 | B | +++ | C | ++ |
| 235 | A | +++ | | |
| 236 | B | +++ | B | +++ |
| 237 | | | A | +++ |
| 238 | B | +++ | B | +++ |
| 239a | B | +++ | | |
| 240a | B | +++ | | |
| 240b | B | +++ | | |
| 241 | B | +++ | | |
| 242 | C | +++ | C | + |
| 243 | B | +++ | C | ++ |
| 244 | B | +++ | C | + |
| 245a | B | +++ | | |
| 245b | B | +++ | | |
| 246a | B | +++ | | |
| 246b | B | +++ | | |
| 247 | A | +++ | | |
| 248 | A | +++ | | |
| 249 | A | +++ | | |
| 250a | A | +++ | | |
| 251a | A | +++ | | |
| 252a | A | +++ | | |
| 253a | B | ++ | | |
| 254a | A | +++ | | |
| 255 | A | +++ | | |
| 256 | A | +++ | | |
| 257 | A | +++ | | |
| 258 | A | +++ | | |
| 259a | A | +++ | | |
| 260 | A | +++ | | |
| 261 | A | +++ | | |
| 262a | A | +++ | | |
| 262b | A | +++ | | |
| 263a | B | +++ | | |
| 263b | A | +++ | | |
| 264 | A | +++ | | |
| 265 | A | +++ | | |
| 266a | A | +++ | | |
| 266b | A | +++ | | |
| 267 | A | +++ | | |
| 268a | A | +++ | | |
| 268b | A | +++ | | |
| 269a | A | +++ | | |
| 270a | A | +++ | | |
| 271a | A | +++ | | |
| 271b | C | +++ | | |
| 272a | A | +++ | | |
| 272b | A | +++ | | |
| 273a | A | +++ | | |
| 273b | A | +++ | | |
| 274a | A | +++ | | |
| 274b | A | +++ | | |
| 275a | A | +++ | | |
| 275b | C | +++ | | |
| 276a | A | +++ | | |
| 276b | A | +++ | | |
| 277 | A | +++ | | |
| 278a | A | +++ | | |
| 278b | A | +++ | | |
| 279 | A | +++ | | |
| 280 | B | +++ | | |
| 281a | A | +++ | | |
| 281b | A | +++ | | |
| 282a | A | +++ | | |
| 282b | A | +++ | | |
| 283a | A | +++ | | |
| 283b | A | +++ | | |
| 284a | A | +++ | | |
| 284b | A | +++ | | |
| 285 | A | +++ | | |
| 286 | A | +++ | | |
| 287 | A | +++ | | |
| 288 | A | +++ | | |
| 289a | A | +++ | | |
| 289b | A | +++ | | |
| 290 | A | +++ | | |
| 291 | A | +++ | | |
| 292 | A | +++ | | |
| 293a | A | +++ | | |
| 294 | A | +++ | | |
| 295 | B | ++ | | |
| 296a | A | +++ | | |
| 297a | A | +++ | | |
| 297b | A | +++ | | |
| 298a | A | +++ | | |
| 299a | A | +++ | | |
| 300a | A | +++ | | |
| 300b | A | +++ | | |
| 301a | A | +++ | | |
| 301b | B | +++ | | |
| 302 | B | +++ | | |
| 303 | A | +++ | | |
| 304 | B | +++ | | |
| 305 | A | +++ | | |
| 306 | A | +++ | | |
| 307a | A | +++ | | |
| 307b | A | +++ | | |
| 308a | A | +++ | | |
| 308b | A | +++ | | |
| 309 | B | ++ | | |
| 310a | A | +++ | | |
| 310b | A | +++ | | |
| 311a | B | +++ | | |
| 311b | B | +++ | | |
| 312 | A | +++ | | |
| 313 | A | +++ | | |
| 314 | B | +++ | | |
| 316a | A | +++ | | |
| 317 | B | +++ | | |
| 318a | A | +++ | | |
| 319a | A | +++ | | |
| 320a | A | +++ | | |
| 320b | A | +++ | | |
| 321a | A | +++ | | |
| 321b | A | +++ | | |
| 322a | A | +++ | | |
| 323a | A | +++ | | |
| 324a | A | +++ | | |
| 325a | A | +++ | | |
| 326a | A | +++ | | |
| 327a | A | +++ | | |
| 328a | A | +++ | | |
| 329a | A | +++ | | |
| 330a | A | +++ | | |
| 331a | A | +++ | | |
| 331b | A | +++ | | |
| 332a | A | +++ | | |
| 332b | A | +++ | | |
| 333a | A | +++ | | |
| 333b | A | +++ | | |
| 334a | A | +++ | | |
| 335a | A | +++ | | |
| 336a | A | +++ | | |
| 337a | A | +++ | | |
| 338a | A | +++ | | |
| 339a | B | +++ | | |
| 340a | A | +++ | | |
| 341a | A | +++ | | |
| 342a | A | +++ | | |
| 343a | A | +++ | | |
| 343b | A | +++ | | |
| 343c | A | +++ | | |
| 343d | A | +++ | | |
| 344a | A | +++ | | |
| 344b | A | +++ | | |
| 344c | A | +++ | | |
| 344d | A | +++ | | |
| 345a | A | +++ | | |
| 346a | A | +++ | | |
| 346b | A | +++ | | |
| 347a | A | +++ | | |
| 347b | A | +++ | | |
| 349a | A | +++ | | |
| 349b | A | +++ | | |
| 350a | A | +++ | | |
| 351a | A | +++ | | |
| 351b | A | +++ | | |
| 352a | A | +++ | | |
| 352b | A | +++ | | |

Notes:
(1) “A”: Yₘᵢₙ < 50%; “B”: 50% ≤ Yₘᵢₙ < 70%; “C”: Yₘᵢₙ ≥ 70%
(2) “+++”: EC₅₀ < 100 nM; “++”: 100 nM ≤ EC₅₀ < 1000 nM; “+”: 1000 nM ≤ EC₅₀ < 10,000 nM

### Example B2

### Western Blotting Based Degradation Assay

BCL-X_{L} degradation in human cancer cell lines is measured using traditional Western blotting methods as well as with the automated “Jess” instrument from ProteinSimple. Cells are seeded at 0.5-1 million cells per well in up to 2 mL of growth media (RPMI+10% FBS) in 6-well plates. The plates are treated with compounds at varying concentrations for up to 5 days. Cells are harvested and lysed using RIPA Buffer (#9806S, Cell Signaling Technology) with Halt Protease & Phosphatase Inhibitor Cocktail (#78441, Thermo Fisher). Protein concentrations of each sample are quantified using Pierce™ BCA Protein Assay Kit (#23225, Thermo Fisher). For the Jess instrument the standard protocol provided by the manufacturer using the Jess Separation Module SM-W004 is followed. For traditional Western blotting, samples resolved on a 4-20% Criterion TGX gel (#5671094, BioRad) and transferred to a PVDF membrane using the Trans-Blot Turbo Transfer system (#1704157, BioRad). Chemiluminescent detection of BCL-X_{L} is performed using a BCL-X_{L} antibody (#2764, Cell Signaling Technology) according to the manufacturer's instructions.

### Example B3

### MOLT-4 Proliferation Assay

MOLT-4 (ATCC CRL-1582) cells are seeded at 4,000 cells per well in 50 μL RPMI 1640 media in black 384-well plates. The plates are pre-spotted with compounds at varying concentrations. After 3 days of incubation at 37° C. with 5% CO₂, cells are assessed using CELLTITERGLO® Cell Viability Assay kit according to manufacturer's instructions (Promega). Relative cell proliferation at each concentration is normalized against the DMSO control.

### Example B4

### Cell Line Proliferation Assay

Human cancer cell lines are seeded at 1,000-4,000 cells per well in 50 μL growth media (following culture conditions recommended by cell line sources) in 384-well plates. The plates are treated with compounds at varying concentrations using a D300e Digital Dispenser (Tecan) and then incubated at 37° C. with 5% CO₂ for 3-7 days. Relative cell viability is assessed using CELLTITERGLO® Cell Viability Assay (#G9243, Promega) according to the manufacturer's instructions or Incucyte SX5 Live-Cell Analysis Instrument (Sartorius) with images recorded at 4-12 hour intervals.

### Example B5

### Apoptosis Assay

Human cancer cell lines are seeded at 1,000-4,000 cells per well in 50 μL growth media (following culture conditions recommended by cell line sources) in 384-well plates. The plates are treated with compounds at varying concentrations using a D300e Digital Dispenser (Tecan) and then incubated at 37° C. with 5% CO₂ for 3-7 days. Relative caspase-3/7 activity is assessed using Caspase-Glo 3/7 Assay System (#G8093, Promega) according to the manufacturer's instructions or Incucyte SX5 Live-Cell Analysis Instrument (Sartorius) with Green Incucyte Caspase-3/7 Dye (#4440, Sartorius) added to the media at between 1:500 and 1:1000 dilution at the time of seeding and images recorded at 4-12 hour intervals. Relative annexin V positivity is assessed using Incucyte SX5 Live-Cell Analysis Instrument (Sartorius) with Orange Incucyte Annexin V Dye (#4759, Sartorius) added to the media at a 1:200 dilution at the time of seeding and images recorded at 4-12 hour intervals.

### Example B6

### Platelet Viability Assay

Human platelet-rich plasma (PRP, SER-PRP-SDS) is obtained from Zenbio. Sodium citrate is used as anticoagulant. To remove any contaminating blood cells, the samples are spun at 200×g at room temperature for 20 minutes in 50 mL falcon tubes, and the supernatant is transferred to a new tube. One hundred microliter PRP are added to each well of a 96-well plate spotted with compounds at varying concentrations. Plates are incubated at 37° C. with 5% CO₂ for 24 hours, and platelet viability are read with CELLTITERGLO® Cell Viability Assay kit according to manufacturer's instructions (Promega). Relative viability at each concentration is normalized against the DMSO control.

### EXEMPLARY EMBODIMENTS

### P01 Embodiments

Embodiment 1. A compound of Formula (I) or (II):

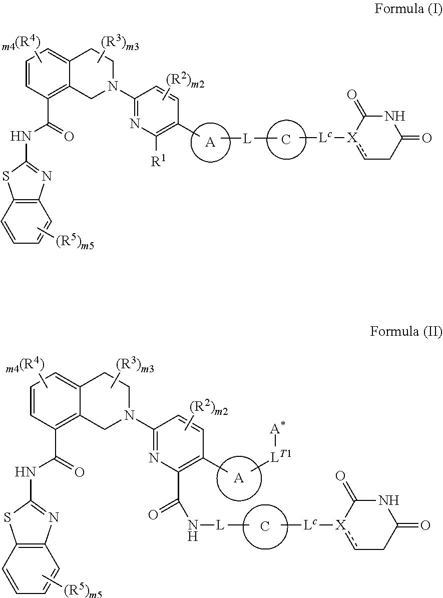

or a pharmaceutically acceptable salt thereof, wherein:
Ring A is selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) phenylene or 5-6 membered heteroarylene, each of which is optionally substituted with 1-3 substituents independently selected from the group consisting of: R^{a} and R^{b};
L^{T1} is a bond or C₁₋₃ alkylene optionally substituted with 1-3 substituents independently selected from the group consisting of: oxo and R^{c}, wherein one CH₂ unit of the C₁₋₃ alkylene is optionally replaced with —O— or —N(R^{d})—;
A* is selected from the group consisting of:
(a) C₃₋₁₅ cycloalkyl or 3-15 membered heterocyclyl, each of which is optionally substituted with 1-6 substituents independently selected from the group consisting of: R^{a} and R^{b};
(b) C₆₋₁₅ aryl or 5-15 membered heteroaryl, each of which is optionally substituted with 1-6 substituents independently selected from the group consisting of: R^{a} and R^{b}; and
(c) H;
R¹ is selected from the group consisting of:
(a) C(O)OH,
(b) C(O)NR^{d}R^{e}, and
(c) C(O)OC₁₋₆ alkyl, wherein the C₁₋₆ alkyl is optionally substituted with 1-3 R^{c};
each R², R³, R⁴, and R⁵ is independently selected from the group consisting of: halo, CN, C₁₋₃ alkyl, C₁₋₃ haloalkyl, C₁₋₃ alkoxy, C₁₋₃ haloalkoxy, OH, and NR^{d}Rₑ;
m2 is 0, 1, or 2;
m3 and m4 are independently 0, 1, 2, or 3;
m5 is 0, 1, 2, 3, or 4;
L is -(L^{A})ₙ₁-, wherein L^{A} and n1 are defined according to (AA) or (BB):
   (AA)
n1 is an integer from 3 to 15; and
each L^{A} is independently selected from the group consisting of: L^{A1} L^{A3}, and L^{A4}, provided that 1-3 occurrences of L^{A} is L^{A4};
   (BB)
n1 is an integer from 0 to 20; and
each L^{A} is independently selected from the group consisting of: L^{A1} and L^{A3};
each L^{A1} is independently selected from the group consisting of: —CH₂—, —CHR^{L}—, and —C(R^{L})₂—;
each L^{A3} is independently selected from the group consisting of: —N(R^{d})—, —N(R^{L})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any N—O, N—N, N—S(O)₀, or O—S(O)₀₋₂ bonds;
wherein each R^{L} is independently selected from the group consisting of: halo; cyano; —OH; —C₁₋₆ alkoxy; —C₁₋₆ haloalkoxy; —NR^{d}R^{e}; C(═O)N(R^{f})₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{e};
Ring C is selected from the group consisting of:

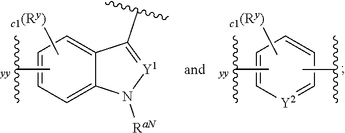

c1 is 0, 1, 2, or 3;
each R^{Y} is independently selected from the group consisting of R^{a} and R^{b};
R^{aN} is H or C₁₋₆ alkyl optionally substituted with 1-3 R^{c};
Y¹ and Y² are independently N, CH, or CR^{Y};
yy represents the point of attachment to L;
X is CH, C, or N;
the [symbol] is a single bond or a double bond;
L^{C} is selected from the group consisting of: a bond, —CH₂—, —CHR^{a}—, —C(R^{a})₂—, —N(R^{d})—, and O, provided that when X is N, then L^{C} is other than O;
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R)₂; and
(q) C₁₋₆ alkyl, C₂₋₆ alkenyl, or C₂₋₆ alkynyl, each optionally substituted with 1-6 R^{e},
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—; —N(H)—; —N(C₁₋₃ alkyl)-; —S(O)₀₋₂—; C(═O); and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R)₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(R^{f})₂; S(O)₁₋₂(C₁₋₆ alkyl); S(O)₁₋₂(C₁₋₆ haloalkyl); S(O)₁₋₂N(R)₂; and C₁₋₆ alkyl optionally substituted with 1-3 R^{h}.
each R^{f} is independently selected from the group consisting of: H and C₁₋₆ alkyl optionally substituted with 1-3 R^{h};
each R^{g} is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂.

Embodiment 2. The compound of Embodiment 1, wherein the compound is a compound of Formula (I) or a pharmaceutically acceptable salt thereof.

Embodiment 3. The compound of Embodiment 1, wherein the compound is a compound of Formula (II) or a pharmaceutically acceptable salt thereof.

Embodiment 4. The compound of any one of Embodiments 1-3, wherein Ring A is a phenylene optionally substituted with 1-3 R^{a}.

Embodiment 5. The compound of any one of Embodiments 1-4, wherein Ring A is

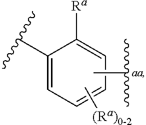

wherein aa represents the point of attachment to L or L^{T1}.

Embodiment 6. The compound of any one of Embodiments 1-5, wherein Ring A is

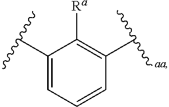

wherein aa represents the point of attachment to L or L^{T1}.

Embodiment 7. The compound of any one of Embodiments 1-5, wherein Ring A is

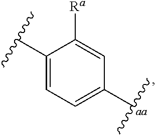

wherein aa represents the point of attachment to L or L^{T1}.

Embodiment 8. The compound of any one of Embodiments 1-3, wherein Ring A is 5-6 membered heteroarylene optionally substituted with 1-3 R^{a}.

Embodiment 9. The compound of any one of Embodiments 1-3 or 8, wherein Ring A is 5-6 membered heteroarylene optionally substituted with 1-2 R^{a}.

Embodiment 10. The compound of any one of Embodiments 1-3 or 8-9, wherein Ring A is 5-membered heteroarylene optionally substituted with 1-2 R^{a}.

Embodiment 11. The compound of any one of Embodiments 1-3 or 8-10, wherein Ring A is pyrazolylene optionally substituted with 1-2 R^{a}.

Embodiment 12. The compound of any one of Embodiments 1-3 or 8-11, wherein Ring A is selected from the group consisting of:

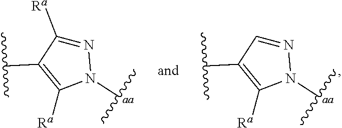

wherein aa represents the point of attachment to L or L^{T1}.

Embodiment 13. The compound of any one of Embodiments 1-3, wherein Ring A is C₃₋₁₀ cycloalkylene optionally substituted with 1-6 R^{a}.

Embodiment 14. The compound of any one of Embodiments 1-13, wherein one R^{a} present on Ring A is C₁₋₃ alkyl optionally substituted with 1-3 F.

Embodiment 15. The compound of any one of Embodiments 1-14, wherein one R^{a} present on Ring A is methyl or CF₃.

Embodiment 16. The compound of any one of Embodiments 1-2 or 4-15, wherein R¹ is C(O)OH.

Embodiment 17. The compound of any one of Embodiments 1-2 or 4-15, wherein R¹ is C(O)NHR^{e}.

Embodiment 18. The compound of any one of Embodiments 1 or 3-15, wherein L^{T1} is C₁₋₃ alkylene.

Embodiment 19. The compound of any one of Embodiments 1, 3-15, or 18, wherein L^{T}1 is —CH₂—.

Embodiment 20. The compound of any one of Embodiments 1, 3-15, or 18-19, wherein A* is H.

Embodiment 21. The compound of any one of Embodiments 1, 3-15, or 18-20, wherein A* is C₃₋₁₅ cycloalkyl or 3-15 membered heterocyclyl, each of which is optionally substituted with 1-6 substituents independently selected from the group consisting of: R^{a} and R^{b}.

Embodiment 22. The compound of any one of Embodiments 1, 3-15, or 18-21, wherein A* is C₃₋₁₅ cycloalkyl optionally substituted with 1-3 R^{a}.

Embodiment 23. The compound of any one of Embodiments 1, 3-15, or 18-22, wherein A* is adamantyl optionally substituted with 1-3 R^{a}.

Embodiment 24. The compound of any one of Embodiments 1-23, wherein m2 is 0.

Embodiment 25. The compound of any one of Embodiments 1-24, wherein m3 is 0.

Embodiment 26. The compound of any one of Embodiments 1-25, wherein m4 is 0.

Embodiment 27. The compound of any one of Embodiments 1-26, wherein m5 is 0.

Embodiment 28. The compound of any one of Embodiments 1-23, wherein m2 is 0; m3 is 0; m4 is 0; and m5 is 0.

Embodiment 29. The compound of any one of Embodiments 1-28, wherein Ring C is

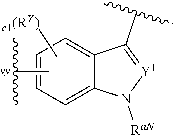

Embodiment 30. The compound of any one of Embodiments 1-29, wherein Ring C is

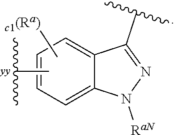

Embodiment 31. The compound of any one of Embodiments 1-30, wherein Ring C is

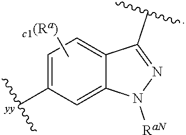

Embodiment 32. The compound of any one of Embodiments 1-30, wherein Ring C is

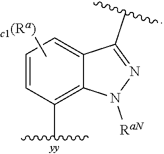

Embodiment 33. The compound of any one of Embodiments 1-30, wherein Ring C is

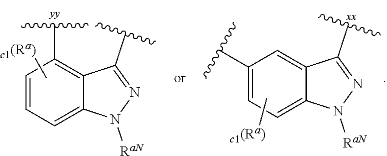

Embodiment 34. The compound of any one of Embodiments 1-28, wherein Ring C is

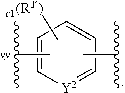

Embodiment 35. The compound of any one of Embodiments 1-28 or 34, wherein Ring C is

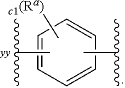

Embodiment 36. The compound of any one of Embodiments 1-28 or 34-35, wherein Ring C is

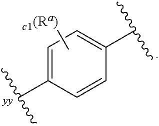

Embodiment 37. The compound of any one of Embodiments 1-28 or 34-35, wherein Ring C is

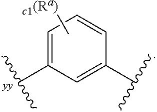

Embodiment 38. The compound of any one of Embodiments 29-37, wherein c1 is 0.

Embodiment 39. The compound of any one of Embodiments 29-33, wherein R^{aN} is C₁₋₃ alkyl.

Embodiment 40. The compound of any one of Embodiments 29-33 or 39, wherein R^{aN} is methyl.

Embodiment 41. The compound of any one of Embodiments 29-40, wherein L^{C} is a bond or N(R^{d}).

Embodiment 42. The compound of any one of Embodiments 29-41, wherein L^{C} is a bond.

Embodiment 43. The compound of any one of Embodiments 29-41, wherein L^{C} is —NH—.

Embodiment 44. The compound of any one of Embodiments 29-43, wherein X is CH.

Embodiment 45. The compound of any one of Embodiments 1-28, wherein the

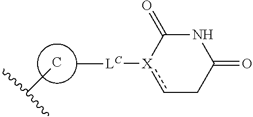

moiety is

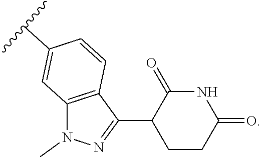 [SMILES: *c1ccc2c(C3CCC(=O)NC3=O)nn(C)c2c1]

Embodiment 46. The compound of any one of Embodiments 1-28, wherein the

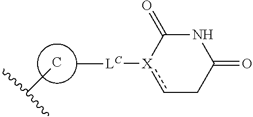

moiety is

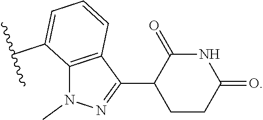 [SMILES: *c1cccc2c(C3CCC(=O)NC3=O)nn(C)c12]

Embodiment 47. The compound of any one of Embodiments 1-28, wherein the

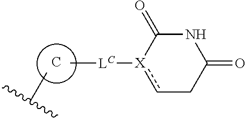

moiety is

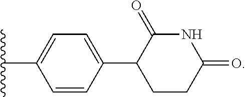 [SMILES: *c1ccc(C2CCC(=O)NC2=O)cc1]

Embodiment 48. The compound of any one of Embodiments 1-28, wherein the

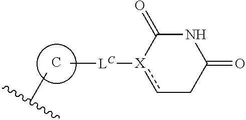

moiety is

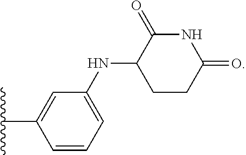 [SMILES: *c1cccc(NC2CCC(=O)NC2=O)c1]

Embodiment 49. The compound of any one of Embodiments 1-48, wherein L is -(L^{A})ₙ₁-; and L^{A} and n1 are defined according to (AA).

Embodiment 50. The compound of any one of Embodiments 1-49, wherein n1 is an integer from 3 to 5.

Embodiment 51. The compound of any one of Embodiments 1-49, wherein n1 is an integer from 5 to 9.

Embodiment 52. The compound of any one of Embodiments 1-49 or 51, wherein n1 is 6, 7, or 8.

Embodiment 53. The compound of any one of Embodiments 1-49, wherein n1 is an integer from 9-12.

Embodiment 54. The compound of any one of Embodiments 1-53, wherein 1-2 occurrences of L^{A} is L^{A4}.

Embodiment 55. The compound of any one of Embodiments 1-54, wherein one occurrence of L^{A} is L^{A4}.

Embodiment 56. The compound of any one of Embodiments 1-54, wherein two occurrences of L^{A} are L^{A4}.

Embodiment 57. The compound of any one of Embodiments 1-56, wherein each L^{A4} is independently selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

Embodiment 58. The compound of any one of Embodiments 1-57, wherein 1-4 occurrences of L^{A} is L^{A3}.

Embodiment 59. The compound of any one of Embodiments 1-58, wherein 1-3 occurrences of L^{A} is L^{A3}.

Embodiment 60. The compound of any one of Embodiments 1-59, wherein 0-1 occurrence of L^{A3} is C(═O); and each remaining occurrence of L^{A3} is independently selected from the group consisting of: —O—; —N(H)—; and —N(C₁₋₃ alkyl)-.

Embodiment 61. The compound of any one of Embodiments 1-60, wherein 2-7 occurrences of L^{A} is L^{A1}.

Embodiment 62. The compound of any one of Embodiments 1-61, wherein 2-5 occurrences of L^{A} is L^{A1}.

Embodiment 63. The compound of any one of Embodiments 1-62, wherein 0-2 occurrences of L^{A1} is —CHR^{L}— or —C(R^{L})₂—; and each remaining occurrence of L^{A1} is —CH₂—.

Embodiment 64. The compound of any one of Embodiments 1-63, wherein each occurrence of L^{A1} is —CH₂—.

Embodiment 65. The compound of any one of Embodiments 1-63, wherein one occurrence of L^{A1} is —CHR^{L}— or —C(R^{L})₂—; and each remaining occurrence of L^{A1} is —CH₂—.

Embodiment 66. The compound of any one of Embodiments 1-65, wherein each R^{L} is independently selected from the group consisting of: —F and —C₁₋₃ alkyl optionally substituted with 1-3 F.

Embodiment 67. The compound of any one of Embodiments 1-49, wherein L is -(L^{A})ₙ₁-, and L^{A} and n1 are defined according to (AA):
n1 is an integer from 5 to 9;
1-2 occurrences of L^{A} is L^{A4};
2-7 occurrences of L^{A} is L^{A1}; and
1-3 occurrences of L^{A} is L^{A3}.

Embodiment 68. The compound of any one of Embodiments 1-49, wherein L is selected from the group consisting of:
(i) -(L^{A3})₀₋₂-(L^{A1})₀₋₅-L^{A4}-(L^{A1})₀₋₅-(L^{A3})₁₋₂-_{bb};
(ii) -(L^{A3})₀₋₂-(L^{A1})₀₋₅-L^{A4}-(L^{A1})₀₋₅-L^{A4}-_{bb};
(iii) -(L^{A3})₀₋₂-(L^{A1})₀₋₅-L^{A4}-(L^{A1})₀₋₅-L^{A3}-L^{A4}-_{bb}; and
(iv) -(L^{A3})₀₋₁-(L^{A1})₂₋₃-L^{A3}-L^{A4}-(L^{A1})₀₋₅-(L^{A3})₁₋₂-_{bb};
provided that L contains 2-7 L^{A1}; and wherein bb represents the point of attachment to Ring C.

Embodiment 69. The compound of any one of Embodiments 1-49, wherein L is:
(L^{A3})₀₋₂-(L^{A1})₂₋₉-L^{A3})₀₋₁-L^{A4}-_{bb},
wherein bb represents the point of attachment to Ring C.

Embodiment 70. The compound of any one of Embodiments 67-69, wherein each L^{A4} is independently selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

Embodiment 71. The compound of any one of Embodiments 67-70, wherein 0-1 occurrence of L^{A1} is —CHR^{L}— or —C(R^{L})₂—; and each remaining occurrence of L^{A1} is —CH₂—.

Embodiment 72. The compound of any one of Embodiments 67-71, wherein 0-1 occurrence of L^{A3} is C(═O); and each remaining occurrence of L^{A3} is independently selected from the group consisting of: —O—; —N(H)—; and —N(C₁₋₃ alkyl)-.

Embodiment 73. The compound of any one of Embodiments 1-49 or 68, wherein L is:
-(L^{A3})₀₋₂-(L^{A1})₀₋₅-L^{A4}-(L^{A1})₀₋₅-(L^{A3})₁₋₂-_{bb};
provided that L contains 2-7 L^{A1}; and wherein bb represents the point of attachment to Ring C.

Embodiment 74. The compound of any one of Embodiments 1-49, 68, or 73, wherein L is:
(L^{A3})₀₋₁-(L^{A1})₀₋₅-L^{A4}-(L^{A1})₀₋₅-(L^{A3})₂-_{bb};
provided that L contains 2-5 L^{A1}; and wherein bb represents the point of attachment to Ring C.

Embodiment 75. The compound of any one of Embodiments 1-49, 68, or 73-74, wherein L is:
-(L^{A3})₀₋₁-(L^{A1})₀₋₅-L^{A4}-(L^{A1})₀₋₅-C(═O)—N(R^{d})-_{bb};
provided that L contains 2-5 L^{A1}; and wherein bb represents the point of attachment to Ring C.

Embodiment 76. The compound of any one of Embodiments 73-75, wherein L is a divalent group of Formula (L-1):

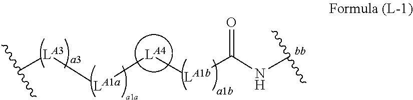

wherein:
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
bb represents the point of attachment to Ring C.

Embodiment 77. The compound of Embodiment 76, wherein a3 is 1.

Embodiment 78. The compound of Embodiments 76 or 77, wherein L^{A3} is —O—.

Embodiment 79. The compound of Embodiment 76, wherein a3 is 0.

Embodiment 80. The compound of any one of Embodiments 76-79, wherein a1a+a1b is 3 or 4.

Embodiment 81. The compound of any one of Embodiments 76-79, wherein a1a+a1b is 2 or 5.

Embodiment 82. The compound of any one of Embodiments 1-49, 68, or 73, wherein L is:
-(L^{A3})₀₋₁-(L^{A1})₀₋₅-L^{A4}-(L^{A1})₀₋₅-L^{A3}-_{bb};
provided that L contains 2-7 L^{A1}; and wherein bb represents the point of attachment to Ring C.

Embodiment 83. The compound of Embodiments 73 or 82, wherein L is a divalent group of Formula (L-2):

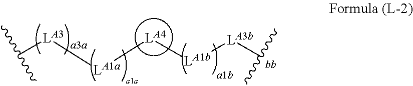

wherein:
a3a is 0 or 1;
L^{A3a} and L^{A3b} are independently selected from the group consisting of: —O—, —N(H)—, and —N(C₁₋₃ alkyl)-;
a1a and a1b are independently integers from 0 to 5, provided that a1a+a1b is from 2 to 7;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
bb represents the point of attachment to Ring C.

Embodiment 84. The compound of Embodiment 83, wherein a3a is 1.

Embodiment 85. The compound of Embodiments 83 or 84, wherein L^{A3}a is —O—.

Embodiment 86. The compound of any one of Embodiments 83-85, wherein L^{A3b} is —N(H)— or —N(C₁₋₃ alkyl)-.

Embodiment 87. The compound of any one of Embodiments 83-85, wherein L^{A3b} is —O—.

Embodiment 88. The compound of any one of Embodiments 83-87, wherein a1a+a1b is 2, 3, or 4.

Embodiment 89. The compound of any one of Embodiments 83-87, wherein a1a+a1b is 5 or 6.

Embodiment 90. The compound of any one of Embodiments 1-49 or 68, wherein L is:
-(L^{A3})₀₋₁-(L^{A1})₀₋₅-L^{A4}-(L^{A1})₀₋₅-L^{A4}-_{bb}; or
-(L^{A3})₀₋₁-(L^{A1})₀₋₅-L^{A4}-(L^{A1})₀₋₅-L^{A3}-L^{A4}-_{bb};
provided that L contains 2-7 L^{A1}; and wherein bb represents the point of attachment to Ring C.

Embodiment 91. The compound of Embodiment 90, wherein L is:
-(L^{A3})₀₋₁-(L^{A1})₀₋₅-L^{A4}-(L^{A1})₀₋₅-(piperazinylene)-_{bb}; or
-(L^{A3})₀₋₁-(L^{A1})₀₋₅-L^{A4}-(L^{A1})₀₋₅-C(═O)-(piperazinylene)-_{bb};
provided that L contains 2-5 L^{A1}; and wherein bb represents the point of attachment to Ring C.

Embodiment 92. The compound of Embodiments 90 or 91, wherein L is a divalent group of Formula (L-3) or (L-4):

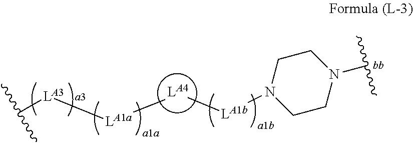

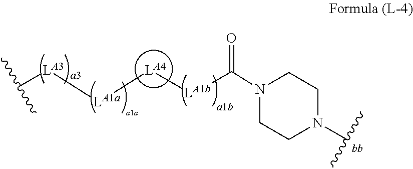

wherein:
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
bb represents the point of attachment to Ring C.

Embodiment 93. The compound of Embodiment 92, wherein a3 is 1.

Embodiment 94. The compound of Embodiments 92 or 93, wherein L^{A3} is —O—.

Embodiment 95. The compound of any one of Embodiments 92-94, wherein a1a+a1b is 3 or 4.

Embodiment 96. The compound of any one of Embodiments 92-94, wherein a1a+a1b is 2.

Embodiment 97. The compound of any one of Embodiments 76-81, 83-89, or 92-96, wherein each occurrence of L^{A1a} and L^{A1b} is —CH₂—.

Embodiment 98. The compound of any one of Embodiments 76-81, 83-89, or 92-96, wherein one occurrence of L^{A1a} is —CHR^{L}— or —C(R^{L})₂—; each remaining occurrence of L^{A1a} is —CH₂—; and each occurrence of L^{A1b} is —CH₂—.

Embodiment 99. The compound of any one of Embodiments 76-81, 83-89, or 92-96, wherein one occurrence of L^{A1b} is —CHR^{L}— or —C(R^{L})₂—; each remaining occurrence of L^{A1b} is —CH₂—; and each occurrence of L^{A1a} is —CH₂—.

Embodiment 100. The compound of any one of Embodiments 1-49 or 68, wherein L is:
-(L^{A3})₀₋₁-(L^{A1})₂₋₃-L^{A3}-L^{A4}-(L^{A1})₀₋₅-(L^{A3})₁₋₂-_{bb};
provided that L contains 2-7 L^{A1}; and wherein bb represents the point of attachment to Ring C.

Embodiment 101. The compound of any one of Embodiments 1-49, 68, or 100, wherein L is:
-(L^{A3})₀₋₁-CH₂CH₂—O-L^{A4}-(L^{A1})₀₋₅-(L^{A3})₁₋₂-_{bb};
wherein bb represents the point of attachment to Ring C.

Embodiment 102. The compound of Embodiments 100 or 101, wherein L is a divalent group of Formula (L-5):

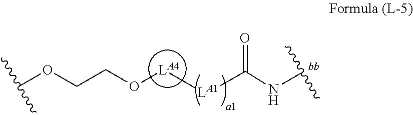

wherein:
a1 is an integer from 0 to 3;
L^{A4} is selected from the group consisting of:
a) C₃₋₁₀ cycloalkylene or 4-10 membered heterocyclylene, each of which is optionally substituted with 1-3 R^{a};
b) phenylene or 5-6 membered heteroarylene, each of which is optionally substituted with 1-3 R^{a}; and
bb represents the point of attachment to Ring C.

Embodiment 103. The compound of Embodiment 102, wherein at is 1.

Embodiment 104. The compound of Embodiments 102 or 103, wherein 0-1 occurrence of L^{A1} is —CHR^{L}— or —C(R^{L})₂—; and each remaining occurrence of L^{A1} is —CH₂—.

Embodiment 105. The compound of any one of Embodiments 73-104, wherein L^{A4} is 4-10 membered heterocyclylene optionally substituted with 1-3 R^{a}.

Embodiment 106. The compound of any one of Embodiments 73-105, wherein L^{A4} is selected from the group consisting of:

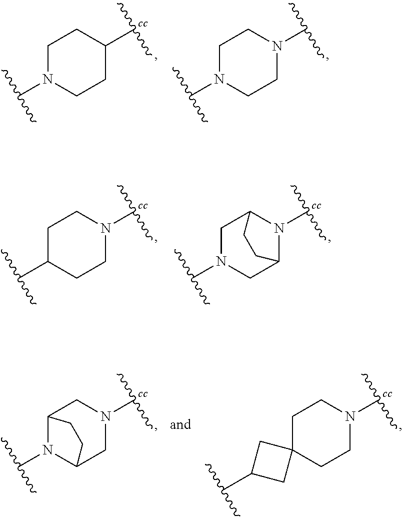 [SMILES: *C1CCN(*)CC1 ; *N1CCN(*)CC1 ; *C1CCN(*)CC1 ; *N1CC2CCC(C1)N2* ; *N1CC2CCC(C1)N2* ; *C1CC2(CCN(*)CC2)C1]

each optionally substituted with 1-3 R^{a} at one or more ring carbon atoms, wherein cc represents the point of attachment to L^{A1b} or L^{A1}.

Embodiment 107. The compound of Embodiments 105 or 106, wherein each R^{a} present on L^{A4} is independently F or C₁₋₃ alkyl optionally substituted with 1-3 F.

Embodiment 108. The compound of any one of Embodiments 73-104, wherein L^{A4} is C₃₋₁₀ cycloalkylene optionally substituted with 1-3 R^{a}.

Embodiment 109. The compound of any one of Embodiments 73-104 or 108, wherein L^{A4} is 1,4-cyclohexylene optionally substituted with 1-3 R^{a}.

Embodiment 110. The compound of any one of Embodiments 73-104, wherein L^{A4} is phenylene or 5-6 membered heteroarylene, each of which is optionally substituted with 1-3 R^{a}.

Embodiment 111. The compound of any one of Embodiments 73-104 or 110, wherein L^{A4} is:
(a) phenylene optionally substituted with 1-3 R^{a};
(b) 1,2-phenylene optionally substituted with 1-3 R^{a}; or
(c) 1,4-phenylene optionally substituted with 1-3 R^{a}.

Embodiment 112. The compound of any one of Embodiments 1-49 or 69, wherein L is:
-(L^{A3})₀₋₁-(L^{A1})₂₋₉-(L)₀₋₁-(piperazinylene)-_{bb},
wherein bb represents the point of attachment to Ring C.

Embodiment 113. The compound of Embodiment 112, wherein L is a divalent group of Formula (L-6):

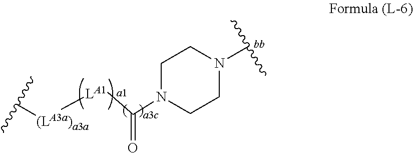

wherein:
L^{A3a} is selected from the group consisting of: —O—, —N(H)—, and —N(C₁₋₃ alkyl)-;
a3a is 0 or 1;
a1 is an integer from 2 to 11;
a3c is 0 or 1; and
bb represents the point of attachment to Ring C.

Embodiment 114. The compound of Embodiment 113, wherein a3a is 1.

Embodiment 115. The compound of Embodiments 113 or 114, wherein L^{A3}a is —O—.

Embodiment 116. The compound of any one of Embodiments 112-115, wherein 0-1 occurrence of L^{A1} is —CHR^{L}— or —C(R^{L})₂—; and each remaining occurrence of L^{A1} is —CH₂—.

Embodiment 117. The compound of any one of Embodiments 1-49 or 73-76, wherein L is selected from the group consisting of:

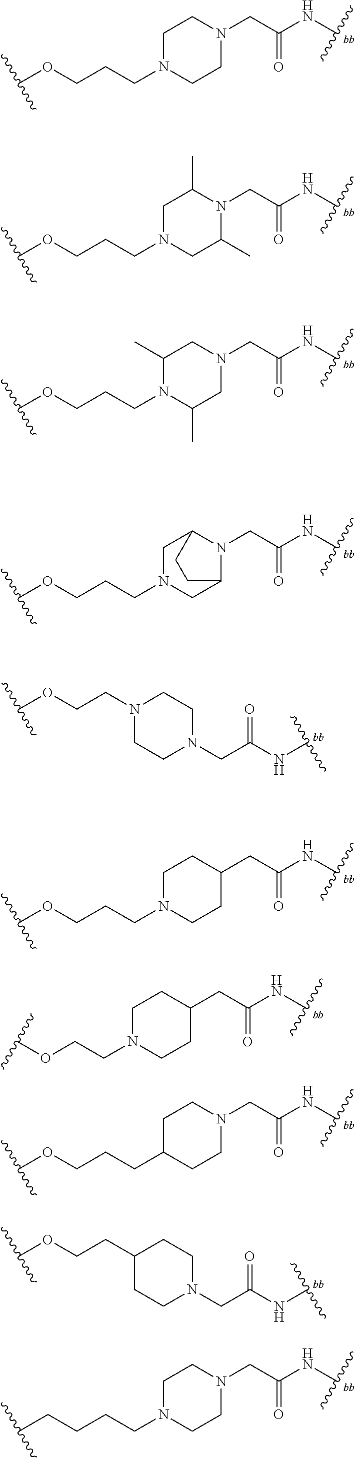

wherein bb represents the point of attachment to Ring C.

Embodiment 118. The compound of any one of Embodiments 1-49 or 73-76, wherein L is selected from the group consisting of.

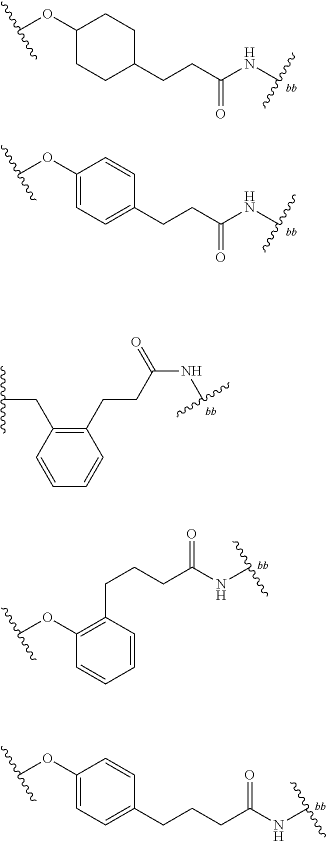

wherein bb represents the point of attachment to Ring C.

Embodiment 119. The compound of any one of Embodiments 1-49, 73, or 82-83, wherein L is

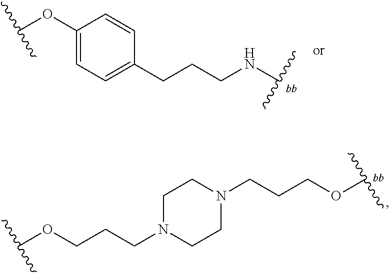

wherein bb represents the point of attachment to Ring C.

Embodiment 120. The compound of any one of Embodiments 1-49 or 90-92, wherein L is

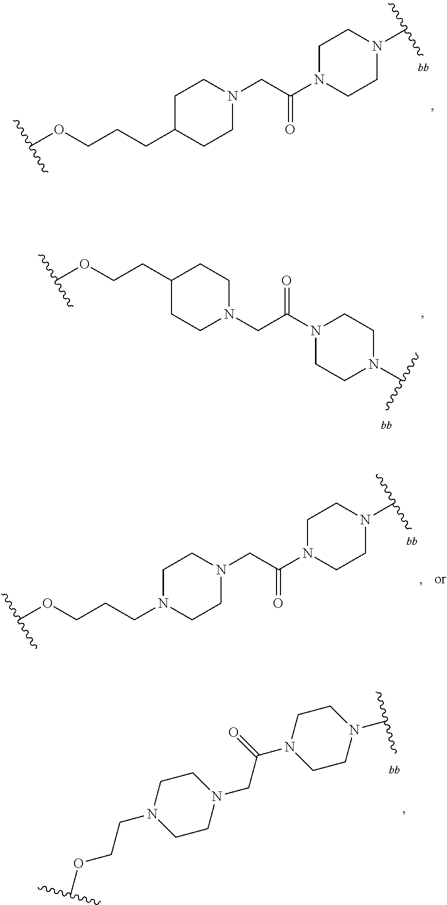

wherein bb represents the point of attachment to Ring C.

Embodiment 121. The compound of any one of Embodiments 1-49 or 100-102, wherein L is

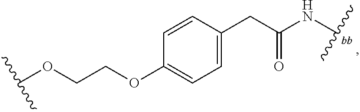

wherein bb represents the point of attachment to Ring C.

Embodiment 122. The compound of any one of Embodiments 1-48, wherein L is -(L^{A})n₁-; and L^{A} and n1 are defined according to (BB).

Embodiment 123. The compound of Embodiment 122, wherein n1 is an integer from 3 to 5.

Embodiment 124. The compound of Embodiment 122, wherein n1 is an integer from 5 to 9.

Embodiment 125. The compound of Embodiment 122, wherein n1 is an integer from 9 to 15.

Embodiment 126. The compound of any one of Embodiments 122-125, wherein 1-4 occurrences of L^{A} is L^{A3}.

Embodiment 127. The compound of any one of Embodiments 122-126, wherein 1-3 occurrences of L^{A} is L^{A3}.

Embodiment 128. The compound of any one of Embodiments 122-127, wherein 2-3 occurrences of L^{A} are L^{A3}.

Embodiment 129. The compound of any one of Embodiments 122-128, wherein 0-1 occurrence of L^{A3} is C(═O); and each remaining occurrence of L^{A3} is independently selected from the group consisting of: —O—; —N(H)—; and —N(C₁₋₃ alkyl)-.

Embodiment 130. The compound of any one of Embodiments 122-129, wherein 0-2 occurrences of L^{A1} is —CHR^{L}— or —C(R^{L})₂—; and each remaining occurrence of L^{A1} is —CH₂—.

Embodiment 131. The compound of any one of Embodiments 1, 3-48, or 122, wherein L^{A} and n1 are defined according to (BB); and n1 is an integer from 3 to 5.

Embodiment 132. The compound of any one of Embodiments 1-48 or 122, wherein L^{A} and n1 are defined according to (BB); and n1 is an integer from 9 to 15.

Embodiment 133. The compound of any one of Embodiments 1-48 or 122, wherein L is:
-(L^{A3})₀₋₂-(L^{A1})₁₋₁₅-(L^{A3})₀₋₂-_{bb},
wherein bb represents the point of attachment to Ring C.

Embodiment 134. The compound of any one of Embodiments 131-133, wherein L contains 2 or 3 L^{A3}.

Embodiment 135. The compound of any one of Embodiments 131-134, wherein 0-1 occurrence of L^{A3} is C(═O); and each remaining occurrence of L^{A3} is independently selected from the group consisting of: —O—; —N(H)—; and —N(C₁₋₃ alkyl)-.

Embodiment 136. The compound of any one of Embodiments 131-135, wherein 0-2 occurrences of L^{A1} is —CHR^{L}— or —C(R^{L})₂—; and each remaining occurrence of L^{A1} is —CH₂—.

Embodiment 137. The compound of any one of Embodiments 131-136, wherein each occurrence of L^{A1} is —CH₂—.

Embodiment 138. The compound of any one of Embodiments 131-136, wherein one occurrence of L^{A1} is —CHR^{L}— or —C(R^{L})₂—; and each remaining occurrence of L^{A1} is —CH₂—.

Embodiment 139. The compound of any one of Embodiments 122-138, wherein each R^{L} is independently selected from the group consisting of: —F and —C₁₋₃ alkyl optionally substituted with 1-3 F.

Embodiment 140. The compound of any one of Embodiments 1-48, 122, or 133, wherein L is:
-(L^{A3})₀₋₁-(L^{A1})₁₋₁₅-(L^{A3})₁₋₂-_{bb},
wherein bb represents the point of attachment to Ring C.

Embodiment 141. The compound of Embodiment 140, wherein L is a divalent group of Formula (L-7):

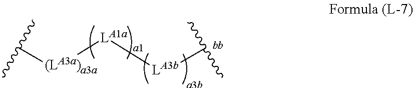

wherein:
L^{A3a} is selected from the group consisting of: —O—, —N(H)—, and —N(C₁₋₃ alkyl)-;
a3a is 0 or 1;
a1 is an integer from 1 to 15;
each L^{A3b} is independently selected from the group consisting of: C(═O), —O—, N(H)—, and —N(C₁₋₃ alkyl)-;
a3b is 1 or 2; and
bb represents the point of attachment to Ring C.

Embodiment 142. The compound of Embodiment 141 wherein -(L^{A3b})_{a3b}- is

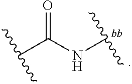 [SMILES: *NC(*)=O]

Embodiment 143. The compound of Embodiment 141, wherein -(L^{A3b})_{a3b}- is —N(H)— or —N(C₁₋₃ alkyl)-.

Embodiment 144. The compound of any one of Embodiments 141-143, wherein 0-2 occurrences of L^{A1} is —CHR^{L}— or —C(R^{L})₂—; and each remaining occurrence of L^{A1} is —CH₂—.

Embodiment 145. The compound of any one of Embodiments 141-144, wherein each occurrence of L^{A1} is —CH₂—.

Embodiment 146. The compound of Embodiment 76, wherein the compound is a compound of Formula (I-A):

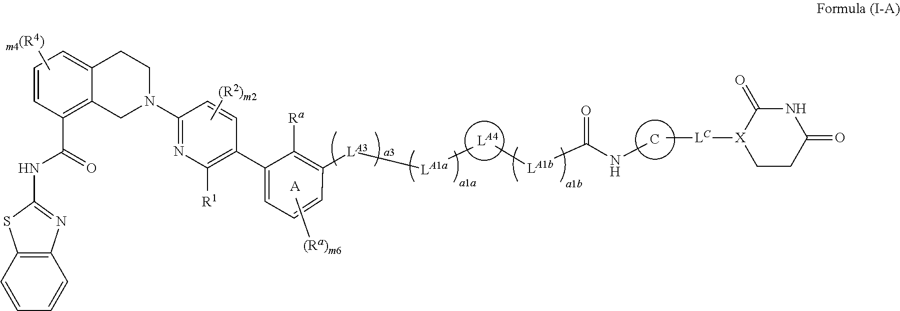

or a pharmaceutically acceptable salt thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

Embodiment 147. The compound of Embodiment 76, wherein the compound is a compound of Formula (I-B):

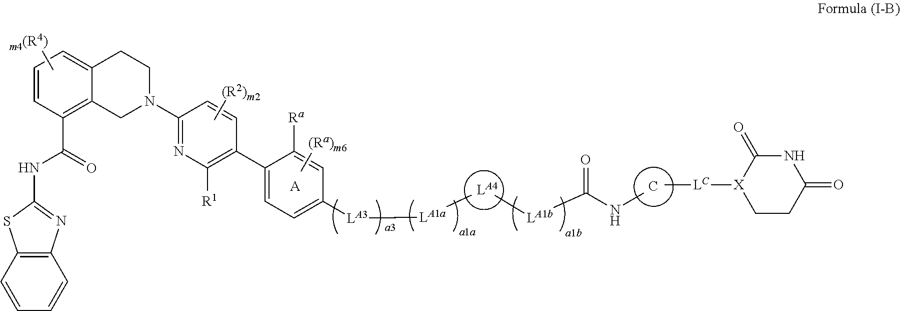

or a pharmaceutically acceptable salt thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

Embodiment 148. The compound of Embodiments 146 or 147, wherein a3 is 1.

Embodiment 149. The compound of any one of Embodiments 146-148, wherein L^{A3} is —O—.

Embodiment 150. The compound of Embodiments 146 or 147, wherein a3 is 0.

Embodiment 151. The compound of Embodiment 83, wherein the compound is a compound of Formula (I-C):

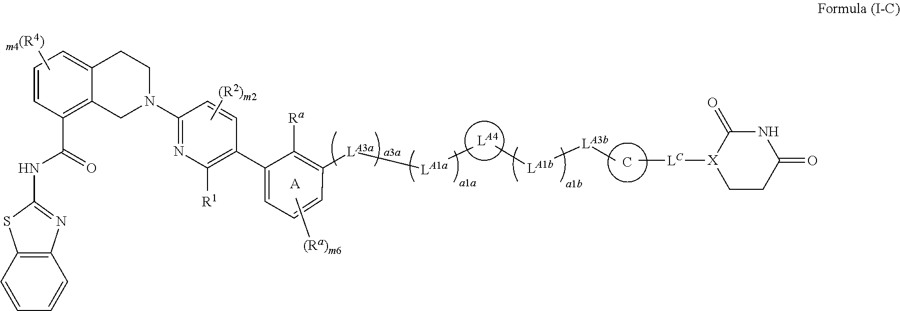

or a pharmaceutically acceptable salt thereof, wherein:
m6 is 0 or 1;
a3a is 0 or 1;
L^{A3a} and L^{A3b} are independently selected from the group consisting of: —O—, —N(H)—, and —N(C₁₋₃ alkyl)-;
a1a and a1b are independently integers from 0 to 5, provided that a1a+a1b is from 2 to 7;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

Embodiment 152. The compound of Embodiment 83, wherein the compound is a compound of Formula (I-D):

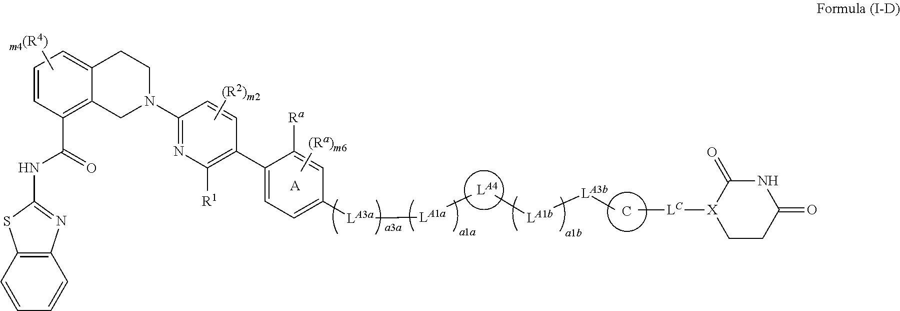

or a pharmaceutically acceptable salt thereof, wherein:
m6 is 0 or 1;
a3a is 0 or 1;
L^{A3a} and L^{A3b} are independently selected from the group consisting of: —O—, —N(H)—, and —N(C₁₋₃ alkyl)-;
a1a and a1b are independently integers from 0 to 5, provided that a1a+a1b is from 2 to 7;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

Embodiment 153. The compound Embodiments 151 or 152, wherein L^{A3b} is —N(H)— or —N(C₁₋₃ alkyl)-.

Embodiment 154. The compound of any one of Embodiments 151-153, wherein a3a is 1.

Embodiment 155. The compound of any one of Embodiments 151-154, wherein L^{A3}a is —O—.

Embodiment 156. The compound of any one of Embodiments 151-153, wherein a3a is 0.

Embodiment 157. The compound of any one of Embodiments 146-156, wherein a1a+a1b is 3.

Embodiment 158. The compound of any one of Embodiments 146-156, wherein a1a+a1b is 4.

Embodiment 159. The compound of any one of Embodiments 146-156, wherein a1a+a1b is 2 or 5.

Embodiment 160. The compound of any one of Embodiments 146-159, wherein each occurrence of L^{A1a} and L^{A1b} is —CH₂—.

Embodiment 161. The compound of any one of Embodiments 146-159, wherein one occurrence of L^{A1a} is —CHR^{L}— or —C(R^{L})₂—; each remaining occurrence of L^{A1a} is —CH₂—; and each occurrence of L^{A1b} is —CH₂—.

Embodiment 162. The compound of any one of Embodiments 146-159, wherein one occurrence of L^{A}b is —CHR^{L}— or —C(R^{L})₂—; each remaining occurrence of L^{A1b} is —CH₂—; and each occurrence of L^{A1a} is —CH₂—.

Embodiment 163. The compound of any one of Embodiments 146-162, wherein L^{A4} is 4-10 membered heterocyclylene optionally substituted with 1-3 R^{a}.

Embodiment 164. The compound of any one of Embodiments 146-163, wherein L^{A4} is selected from the group consisting of.

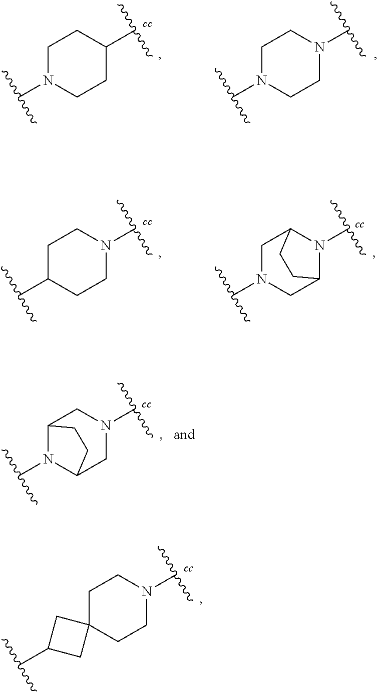 [SMILES: *C1CCN(*)CC1 ; *N1CCN(*)CC1 ; *C1CCN(*)CC1 ; *N1CC2CCC(C1)N2* ; *N1CC2CCC(C1)N2* ; *C1CC2(CCN(*)CC2)C1]

each optionally substituted with 1-3 R^{a} at one or more ring carbon atoms, wherein cc represents the point of attachment to L^{A1b}.

Embodiment 165. The compound of Embodiments 163 or 164, wherein each R^{a} present on L^{A4} is independently a C₁₋₃ alkyl optionally substituted with 1-3 F.

Embodiment 166. The compound of any one of Embodiments 146-162, wherein L^{A4} is C₃₋₁₀ cycloalkylene optionally substituted with 1-3 R^{a}.

Embodiment 167. The compound of any one of Embodiments 146-162 or 166, wherein L^{A4} is 1,4-cyclohexylene optionally substituted with 1-3 R^{a}.

Embodiment 168. The compound of any one of Embodiments 146-162, wherein L^{A4} is phenylene or 5-6 membered heteroarylene, each of which is optionally substituted with 1-3 R^{a}.

Embodiment 169. The compound of any one of Embodiments 146-162 or 168, wherein L^{A4} is 1,4-phenylene optionally substituted with 1-3 R^{a}.

Embodiment 170. The compound of any one of Embodiments 146-162 or 168, wherein L^{A4} is 1,2-phenylene or 1,3-phenylene, each of which is optionally substituted with 1-3 R^{a}.

Embodiment 171. The compound of any one of Embodiments 146-170, wherein m6 is 0.

Embodiment 172. The compound of any one of Embodiments 146-171, wherein the R^{a} present on Ring A is C₁₋₃ alkyl optionally substituted with 1-3-F.

Embodiment 173. The compound of any one of Embodiments 146-172, wherein R¹ is C(O)OH.

Embodiment 174. The compound of Embodiment 141, wherein a3a is 0; and the compound is a compound of Formula (II-A):

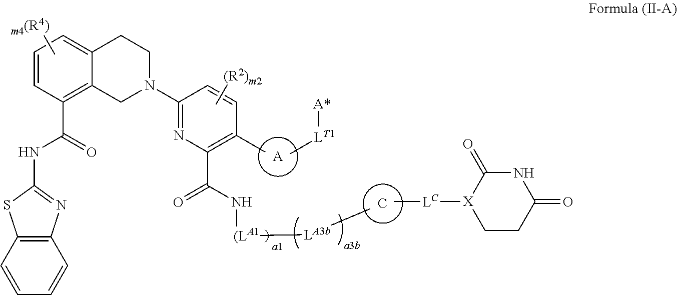

or a pharmaceutically acceptable salt thereof, wherein:
Ring A is phenylene or 5-6 membered heteroarylene, each of which is optionally substituted with 1-3 R^{a};
L^{T1} is a bond or CH₂;
A* is H or C₃₋₁₅ cycloalkyl optionally substituted with 1-3 R^{a};
L^{A3b} is selected from the group consisting of: C(═O), —O—, N(H)—, and —N(C₁₋₃ alkyl)-;
a3b is 1 or 2; and
a1 is an integer from 1 to 15.

Embodiment 175. The compound of Embodiment 174, wherein Ring A is 5-membered heteroarylene optionally substituted with 1-2 R^{a}.

Embodiment 176. The compound of Embodiments 174 or 175, wherein Ring A is selected from the group consisting of:

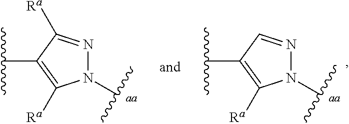

wherein aa represents the point of attachment to L^{T1}.

Embodiment 177. The compound of any one of Embodiments 174-176, wherein L^{T1} is CH₂.

Embodiment 178. The compound of any one of Embodiments 174-177, wherein A* is H.

Embodiment 179. The compound of any one of Embodiments 174-177, wherein A* is C₃₋₁₅ cycloalkyl optionally substituted with 1-3 R^{a}.

Embodiment 180. The compound of any one of Embodiments 174-177 or 179, wherein A* is adamantyl optionally substituted with 1-3 R^{a}.

Embodiment 181. The compound of any one of Embodiments 174-180, wherein at is an integer from 1 to 3.

Embodiment 182. The compound of any one of Embodiments 174-180, wherein at is an integer from 4 to 6.

Embodiment 183. The compound of any one of Embodiments 174-180, wherein at is an integer from 7 to 9.

Embodiment 184. The compound of any one of Embodiments 174-180, wherein at is an integer from 10 to 15.

Embodiment 185. The compound of any one of Embodiments 174-184, wherein -(L^{A3b})_{a3b}- is

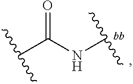

wherein bb represents the point of attachment to Ring C.

Embodiment 186. The compound of any one of Embodiments 146-185, wherein m2 is 0.

Embodiment 187. The compound of any one of Embodiments 146-186, wherein m4 is 0.

Embodiment 188. The compound of any one of Embodiments 146-187, wherein Ring C is

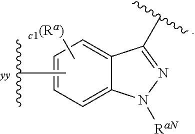

Embodiment 189. The compound of any one of Embodiments 146-188, wherein Ring C is

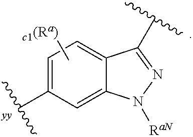

Embodiment 190. The compound of any one of Embodiments 146-188, wherein Ring C is

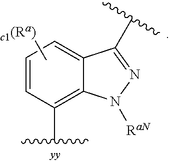

Embodiment 191. The compound of any one of Embodiments 146-190, wherein R^{aN} is C₁₋₃ alkyl.

Embodiment 192. The compound of any one of Embodiments 146-191, wherein R^{aN} is methyl.

Embodiment 193. The compound of any one of Embodiments 146-187, wherein Ring C is

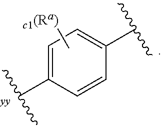

Embodiment 194. The compound of any one of Embodiments 146-187, wherein Ring C is

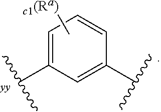

Embodiment 195. The compound of any one of Embodiments 146-194, wherein c1 is 0.

Embodiment 196. The compound of any one of Embodiments 146-195, wherein L^{C} is a bond.

Embodiment 197. The compound of any one of Embodiments 146-195, wherein L^{C} is —NH—.

Embodiment 198. The compound of any one of Embodiments 146-197, wherein X is CH.

Embodiment 199. The compound of any one of Embodiments 146-187, wherein the

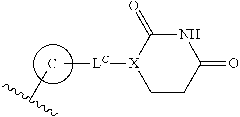

moiety is

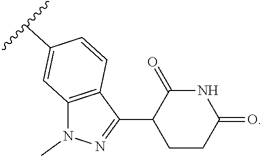 [SMILES: *c1ccc2c(C3CCC(=O)NC3=O)nn(C)c2c1]

Embodiment 200. The compound of any one of Embodiments 146-187, wherein the

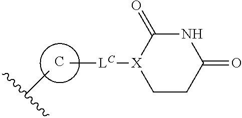

moiety is

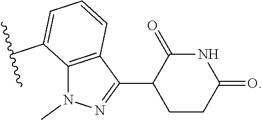 [SMILES: *c1cccc2c(C3CCC(=O)NC3=O)nn(C)c12]

Embodiment 201. The compound of any one of Embodiments 146-187, wherein the

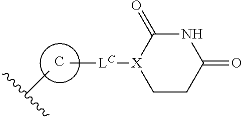

moiety is

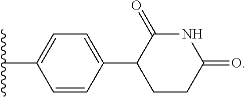 [SMILES: *c1ccc(C2CCC(=O)NC2=O)cc1]

Embodiment 202. The compound of any one of Embodiments 146-187, wherein the

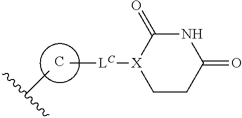

moiety is

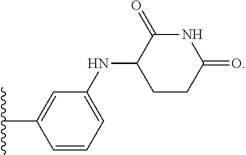 [SMILES: *c1cccc(NC2CCC(=O)NC2=O)c1]

Embodiment 203. The compound of any one of Embodiments 1-202, wherein the compound is selected from the group consisting of Compound No. 101, 102, 103, 104, 105, 106, 107, 108, 109, 110, 111, 112, 113, 114, 115, 116, 116a, 117, 117a, 118, 119, 120, 121, 122, 123, 124, 124a, 125, 125a, 126, 127, 128, 129, 130, 131, 131a, 132, 133, 134, 135, 136, 137, 138, 138a, 139, 140, 141, 142, 143, 144, 145, 146, 146a, 147, 147a, 148, 148a, 149, 150, 151, 152, 153, 154, 154a, 155, and 156 as depicted in Table C1, or a pharmaceutically acceptable salt thereof.

Embodiment 204. A pharmaceutical composition comprising a compound of any one of Embodiments 1-203, or a pharmaceutically acceptable salt thereof, and a pharmaceutically acceptable excipient.

Embodiment 205. A Bcl-xL protein non-covalently bound with a compound of any one of Embodiments 1-203, or a pharmaceutically acceptable salt thereof.

Embodiment 206. A ternary complex comprising a Bcl-xL protein, a compound of in any one of Embodiments 1-203, or a pharmaceutically acceptable salt thereof, and a CRBN protein.

Embodiment 207. A method for treating a cancer in a subject in need thereof, the method comprising administering to the subject a therapeutically effective amount of a compound of any one of Embodiments 1-203, or a pharmaceutically acceptable salt thereof, or a pharmaceutical composition according to Embodiment 204.

Embodiment 208. A method for inducing degradation of a Bcl-xL protein in a mammalian cell, the method comprising contacting the mammalian cell with an effective amount of a compound of any one of Embodiments 1-203, or a pharmaceutically acceptable salt thereof.

Embodiment 209. The method of Embodiment 208, wherein the contacting occurs in vivo.

Embodiment 210. The method of Embodiment 208, wherein the contacting occurs in vitro.

Embodiment 211. The method of any one of Embodiments 208-210, wherein the mammalian cell is a mammalian cancer cell.

### P06 Embodiments

Embodiment 1. A compound of Formula (I):

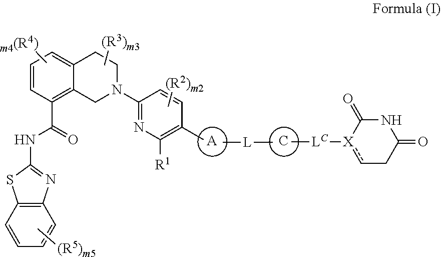

or a pharmaceutically acceptable salt thereof, wherein:
Ring A is selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) phenylene or 5-6 membered heteroarylene, each of which is optionally substituted with 1-3 substituents independently selected from the group consisting of: R^{a} and R^{b};
R¹ is selected from the group consisting of:
(a) C(O)OH,
(b) C(O)NR^{d}R^{e}, and
(c) C(O)OC₁₋₆ alkyl, wherein the C₁₋₆ alkyl is optionally substituted with 1-3 R^{c};
each R², R³, R⁴, and R⁵ is independently selected from the group consisting of: halo, CN, C₁₋₃ alkyl, C₁₋₃ haloalkyl, C₁₋₃ alkoxy, C₁₋₃ haloalkoxy, OH, and NR^{d}R^{e};
m2 is 0, 1, or 2;
m3 and m4 are independently 0, 1, 2, or 3;
m5 is 0, 1, 2, 3, or 4;
L is -(L^{A})ₙ₁-, wherein L^{A} and n1 are defined according to (AA) or (BB):
   (AA)
n1 is an integer from 3 to 15; and
each L^{A} is independently selected from the group consisting of: L^{A1} L^{A3}, and L^{A4}, provided that 1-3 occurrences of L^{A} is L^{A4};
   (BB)
n1 is an integer from 0 to 20; and
each L^{A} is independently selected from the group consisting of: L^{A1} and L^{A3};
each L^{A1} is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any N—O, 0-0, N—N, N—S(O)o, or O—S(O)₀₋₂ bonds;
wherein each R^{L} is independently selected from the group consisting of: halo; cyano; —OH; —C₁₋₆ alkoxy; —C₁₋₆ haloalkoxy; —NR^{d}R^{e}; C(═O)N(R^{f})₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{c};
Ring C is selected from the group consisting of:

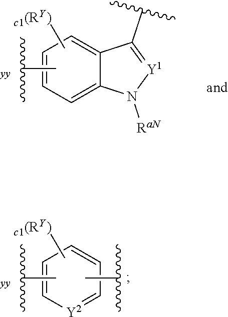

c1 is 0, 1, 2, or 3;
each R^{Y} is independently selected from the group consisting of R^{a} and R^{b};
R^{a}N is H or C₁₋₆ alkyl optionally substituted with 1-3 R^{c};
Y¹ and Y² are independently N, CH, or CR^{Y};
yy represents the point of attachment to L;
X is CH, C, or N;
the [symbol] is a single bond or a double bond;
L^{C} is selected from the group consisting of: a bond, —CH₂—, —CHR^{a}—, —C(R^{a})₂—, —N(R^{d})—, and O, provided that when X is N, then L^{C} is other than O;
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{e};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—; —N(H)—; —N(C₁₋₃ alkyl)-; —S(O)₀₋₂—; C(═O); and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(R^{f})₂; S(O)₁₋₂(C₁₋₆ alkyl); S(O)₁₋₂(C₁₋₆ haloalkyl); S(O)₁₋₂N(R)₂; and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂.

Embodiment 2. The compound of Embodiment 1, wherein Ring A is a phenylene optionally substituted with 1-3 R^{a}.

Embodiment 3. The compound of Embodiment 1 or 2, wherein Ring A is

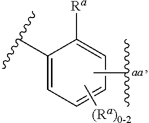

wherein aa represents the point of attachment to L.

Embodiment 4. The compound of any one of Embodiments 1-3, wherein Ring A is

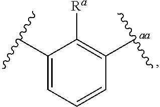

wherein aa represents the point of attachment to L.

Embodiment 5. The compound of any one of Embodiments 1-3, wherein Ring A is

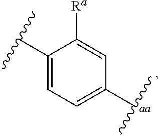

wherein aa represents the point of attachment to L.

Embodiment 6. The compound of Embodiment 1, wherein Ring A is 5-6 membered heteroarylene optionally substituted with 1-3 R^{a}.

Embodiment 7. The compound of any one of Embodiments 1-6, wherein one R^{a} present on Ring A is C₁₋₃ alkyl optionally substituted with 1-3 F.

Embodiment 8. The compound of any one of Embodiments 1-7, wherein one R^{a} present on Ring A is methyl or CF₃.

Embodiment 9. The compound of any one of Embodiments 1-8, wherein R¹ is C(O)OH.

Embodiment 10. The compound of any one of Embodiments 1-9, wherein m2 is 0.

Embodiment 11. The compound of any one of Embodiments 1-10, wherein m3 is 0.

Embodiment 12. The compound of any one of Embodiments 1-11, wherein m4 is 0.

Embodiment 13. The compound of any one of Embodiments 1-12, wherein m5 is 0.

Embodiment 14. The compound of any one of Embodiments 1-9, wherein m2 is 0; m3 is 0; m4 is 0; and m5 is 0.

Embodiment 15. The compound of any one of Embodiments 1-14, wherein Ring C is

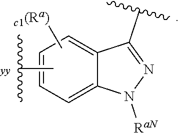

Embodiment 16. The compound of any one of Embodiments 1-15, wherein Ring C is

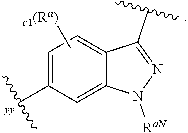

Embodiment 17. The compound of any one of Embodiments 1-15, wherein Ring C is

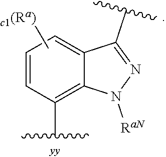

Embodiment 18. The compound of any one of Embodiments 15-17, wherein c1 is 0.

Embodiment 19. The compound of any one of Embodiments 15-18, wherein R^{aN} is C₁₋₃ alkyl.

Embodiment 20. The compound of any one of Embodiments 15-19, wherein R^{aN} is methyl.

Embodiment 21. The compound of any one of Embodiments 15-20, wherein L^{C} is a bond.

Embodiment 22. The compound of any one of Embodiments 15-21, wherein X is CH.

Embodiment 23. The compound of any one of Embodiments 1-15, wherein the

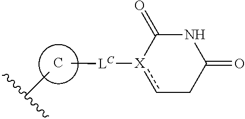

moiety is

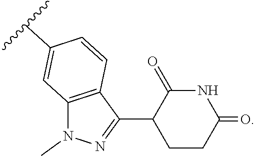 [SMILES: *c1ccc2c(C3CCC(=O)NC3=O)nn(C)c2c1]

Embodiment 24. The compound of any one of Embodiments 1-15, wherein the

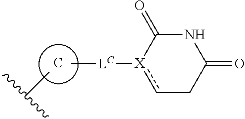

moiety is

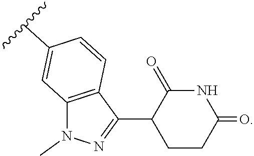 [SMILES: *c1ccc2c(C3CCC(=O)NC3=O)nn(C)c2c1]

Embodiment 25. The compound of any one of Embodiments 1-24, wherein L is -(L^{A})ₙ₁-; and L^{A} and n1 are defined according to (AA).

Embodiment 26. The compound of any one of Embodiments 1-25, wherein n1 is an integer from 3 to 5.

Embodiment 27. The compound of any one of Embodiments 1-25, wherein n1 is an integer from 5 to 9.

Embodiment 28. The compound of any one of Embodiments 1-25 or 27, wherein n1 is 6, 7, or 8.

Embodiment 29. The compound of any one of Embodiments 1-25, wherein n1 is an integer from 9 to 12.

Embodiment 30. The compound of any one of Embodiments 1-29, wherein 1-2 occurrences of L^{A} is L^{A4}.

Embodiment 31. The compound of any one of Embodiments 1-29, wherein one occurrence of L^{A} is L^{A4}.

Embodiment 32. The compound of any one of Embodiments 1-29, wherein two occurrences of L^{A} are L^{A4}.

Embodiment 33. The compound of any one of Embodiments 1-32, wherein each L^{A4} is independently selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

Embodiment 34. The compound of any one of Embodiments 1-33, wherein 1-4 occurrences of L^{A} is L^{A3}.

Embodiment 35. The compound of any one of Embodiments 1-34, wherein 1-3 occurrences of L^{A} is L^{A3}.

Embodiment 36. The compound of any one of Embodiments 1-35, wherein 0-1 occurrence of L^{A3} is C(═O); and each remaining occurrence of L^{A3} is independently selected from the group consisting of: —O—; —N(H)—; and —N(C₁₋₃ alkyl)-.

Embodiment 37. The compound of any one of Embodiments 1-36, wherein 2-7 occurrences of L^{A} is L^{A1}.

Embodiment 38. The compound of any one of Embodiments 1-37, wherein 2-5 occurrences of L^{A} is L^{A1}.

Embodiment 39. The compound of any one of Embodiments 1-38, wherein 0-2 occurrences of L^{A1} is —CHR^{L}— or —C(R^{L})₂—; and each remaining occurrence of L^{A1} is —CH₂—.

Embodiment 40. The compound of any one of Embodiments 1-39, wherein each occurrence of L^{A1} is —CH₂—.

Embodiment 41. The compound of any one of Embodiments 1-39, wherein one occurrence of L^{A1} is —CHR^{L}— or —C(R^{L})₂—; and each remaining occurrence of L^{A1} is —CH₂—.

Embodiment 42. The compound of any one of Embodiments 1-41, wherein each R^{L} is independently selected from the group consisting of: —F and —C₁₋₃ alkyl optionally substituted with 1-3 F.

Embodiment 43. The compound of any one of Embodiments 1-25, wherein L is -(L^{A})ₙ₁-, and L^{A} and n1 are defined according to (AA):
n1 is an integer from 5 to 9;
1-2 occurrences of L^{A} is L^{A4};
2-7 occurrences of L^{A} are L^{A1}; and
1-3 occurrences of L^{A} is L^{A3}.

Embodiment 44. The compound of any one of Embodiments 1-25, wherein L is selected from the group consisting of:
(i) -(L^{A3})₀₋₂-(L^{A1})₀₋₅-L^{A4}-(L^{A1})₀₋₅-(L^{A3})₁₋₂-_{bb};
(ii) -(L^{A3})₀₋₂-(L^{A1})₀₋₅-L^{A4}-(L^{A1})₀₋₅-L^{A4}-_{bb};
(iii) -(L^{A3})₀₋₂-(L^{A1})₀₋₅-L^{A4}-(A)₀₋₅-L^{A3}-L^{A4}-_{bb}; and
(iv) -(L^{A3})₀₋₁-(L^{A1})₂₋₃-L^{A3}-L^{A4}-(L^{A1})₀₋₅-(L^{A3})₁₋₂-_{bb};
provided that L contains 1-7 L^{A1}; and wherein bb represents the point of attachment to Ring C.

Embodiment 45. The compound of any one of Embodiments 1-25, wherein L is:
-(L^{A3})₀₋₂-(L^{A1})₂₋₉-(L^{A3})₀₋₁-L^{A4}-_{bb},
wherein bb represents the point of attachment to Ring C.

Embodiment 46. The compound of Embodiments 43-45, wherein each L^{A4} is independently selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

Embodiment 47. The compound of any one of Embodiments 43-46, wherein 0-1 occurrence of L^{A1} is —CHR^{L}— or —C(R^{L})₂—; and each remaining occurrence of L^{A1} is —CH₂—.

Embodiment 48. The compound of any one of Embodiments 43-47, wherein 0-1 occurrence of L^{A3} is C(═O); and each remaining occurrence of L^{A3} is independently selected from the group consisting of: —O—; —N(H)—; and —N(C₁₋₃ alkyl)-.

Embodiment 49. The compound of any one of Embodiments 1-25, wherein L is:
-(L^{A3})₀₋₂-(L^{A1})₀₋₅-L^{A4}-(L^{A1})₀₋₅-(L^{A3})₁₋₂-_{bb};
provided that L contains 1-7 L^{A1}; and wherein bb represents the point of attachment to Ring C.

Embodiment 50. The compound of any one of Embodiments 1-25 or 49, wherein L is:
-(L^{A3})₀₋₁-(L^{A1})₀₋₅-L^{A4}-(L^{A1})₀₋₅-(L^{A3})₂-_{bb};
provided that L contains 1-5 L^{A1}; and wherein bb represents the point of attachment to Ring C.

Embodiment 51. The compound of any one of Embodiments 1-25 or 49-50, wherein L is:
-(L^{A3})₀₋₁-(L^{A1})₀₋₅-L^{A4}-(L^{A1})₀₋₅-C(═O)—N(R^{d})-_{bb};
provided that L contains 1-5 L^{A1}; and wherein bb represents the point of attachment to Ring C.

Embodiment 52. The compound of any one of Embodiments 49-51, wherein L is a divalent group of Formula (L-1):

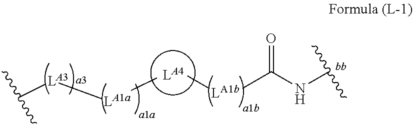

wherein:
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 1 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
bb represents the point of attachment to Ring C.

Embodiment 53. The compound of Embodiment 52, wherein a3 is 1.

Embodiment 54. The compound of Embodiments 52 or 53, wherein L^{A3} is —O—.

Embodiment 55. The compound of Embodiment 52, wherein a3 is 0.

Embodiment 56. The compound of any one of Embodiments 52-55, wherein a1a+a1b is 3 or 4.

Embodiment 57. The compound of any one of Embodiments 52-55, wherein a1a+a1b is 2 or 5.

Embodiment 58. The compound of any one of Embodiments 1-25 or 49, wherein L is:
-(L^{A3})₀₋₁-(L^{A1})₀₋₅-L^{A4}-(L^{A1})₀₋₅-L^{A3}-_{bb};
provided that L contains 2-7 L^{A1}; and wherein bb represents the point of attachment to Ring C.

Embodiment 59. The compound of Embodiments 49 or 58, wherein L is a divalent group of Formula (L-2):

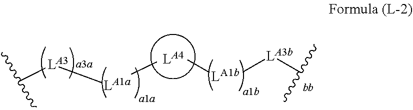

wherein:
a3a is 0 or 1;
L^{A3a} and L^{A3b} are independently selected from the group consisting of: —O—, —N(H)—, and —N(C₁₋₃ alkyl)-;
a1a and a1b are independently integers from 0 to 5, provided that a1a+a1b is from 2 to 7;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
bb represents the point of attachment to Ring C.

Embodiment 60. The compound of Embodiment 59, wherein a3a is 1.

Embodiment 61. The compound of Embodiments 59 or 60, wherein L^{A3}a is —O—.

Embodiment 62. The compound of any one of Embodiments 59-61, wherein L^{A3b} is —N(H)— or —N(C₁₋₃ alkyl)-.

Embodiment 63. The compound of any one of Embodiments 59-61, wherein L^{A3b} is —O—.

Embodiment 64. The compound of any one of Embodiments 59-63, wherein a1a+a1b is 2, 3, or 4.

Embodiment 65. The compound of any one of Embodiments 59-63, wherein a1a+a1b is 5 or 6.

Embodiment 66. The compound of any one of Embodiments 1-25, wherein L is:
-(L^{A3})₀₋₁-(L^{A1})₀₋₅-L^{A4}-(L^{A1})₀₋₅-L^{A4}-_{bb}; or
-(L^{A3})₀₋₁-(L^{A1})₀₋₅-L^{A}-(L^{A1})₀₋₅-L^{A3}-L^{A4}-_{bb};
provided that L contains 2-7 L^{A1}; and wherein bb represents the point of attachment to Ring C.

Embodiment 67. The compound of Embodiment 66, wherein L is:
-(L^{A3})₀₋₁-(L^{A1})₀₋₅-L^{A4}-(L^{A1})₀₋₅-(piperazinylene)-_{bb};
-(L^{A3})₀₋₁-(L^{A1})₀₋₅-L^{A4}-(L^{A1})₀₋₅-(piperidinylene)-_{bb}; or
-(L^{A3})₀₋₁-(L^{A1})₀₋₅-L^{A4}-(L^{A1})₀₋₅-C(═O)-(piperazinylene)-_{bb};
provided that L contains 2-5 L^{A1};
wherein the piperazinylene and piperidinylene are each optionally substituted with 1-3 R^{a}, and
wherein bb represents the point of attachment to Ring C.

Embodiment 68. The compound of Embodiment 66, wherein L is a divalent group of Formula (L-3), (L-3a), (L-3b), or (L-4):

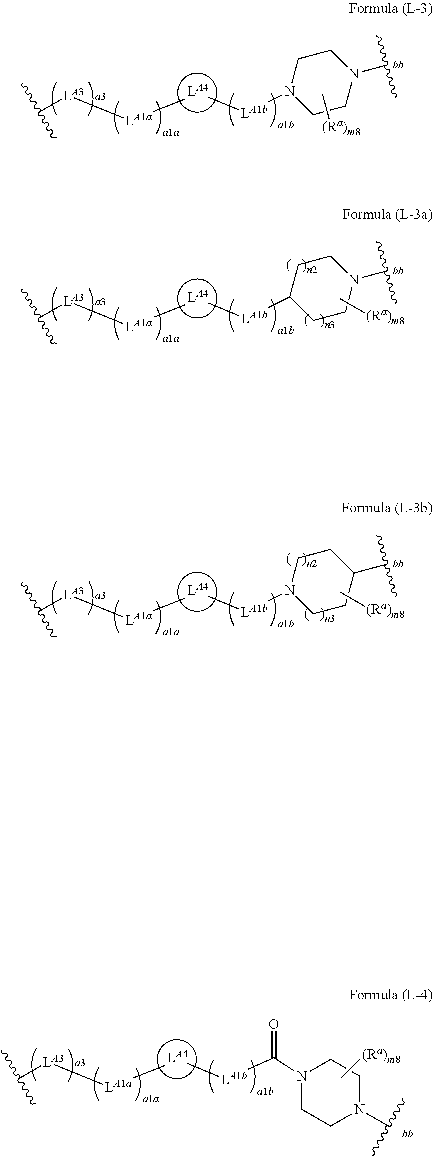

wherein:
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1}b are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
n2 and n3 are independently 0, 1, or 2;
m8 is 0, 1, or 2;
L^{A4b} is 7-10 membered bicyclic nitrogen-containing heterocyclylene optionally substituted with 1-3 R^{a}; and
bb represents the point of attachment to Ring C.

Embodiment 69. The compound of Embodiment 68, wherein a3 is 1.

Embodiment 70. The compound of Embodiments 68 or 69, wherein L^{A3} is —O—.

Embodiment 71. The compound of any one of Embodiments 68-70, wherein a1a+a1b is 3 or 4.

Embodiment 72. The compound of any one of Embodiments 68-70, wherein a1a+a1b is 2.

Embodiment 73. The compound of any one of Embodiments 52-57, 59-65, or 68-72, wherein each occurrence of L^{A1a} and L^{A1b} is —CH₂—.

Embodiment 74. The compound of any one of Embodiments 52-57, 59-65, or 68-72, wherein one occurrence of L^{A1a} is —CHR^{L}— or —C(R^{L})₂—; each remaining occurrence of L^{A1a} is —CH₂—; and each occurrence of L^{A1b} is —CH₂—.

Embodiment 75. The compound of any one of Embodiments 52-57, 59-65, or 68-72, wherein one occurrence of L^{A1b} is —CHR^{L}— or —C(R^{L})₂—; each remaining occurrence of L^{A1b} is —CH₂—; and each occurrence of L^{A1a} is —CH₂—.

Embodiment 76. The compound of any one of Embodiments 49-75, wherein L^{A4} is 4-10 membered heterocyclylene optionally substituted with 1-3 R^{a}.

Embodiment 77. The compound of any one of Embodiments 49-75, wherein L^{A4} is 4-10 membered nitrogen-containing heterocyclylene optionally substituted with 1-3 R^{a}.

Embodiment 78. The compound of any one of Embodiments 49-77, wherein L^{A4} is selected from the group consisting of:

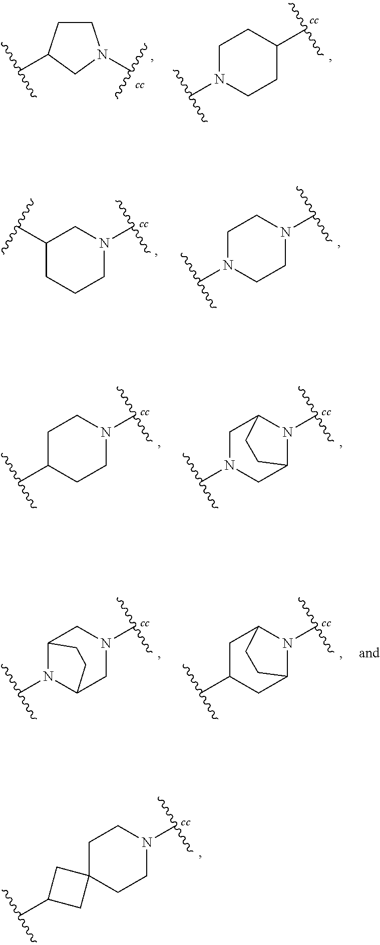

each optionally substituted with 1-3 R^{a} at one or more ring carbon atoms, wherein cc represents the point of attachment to L^{A1b}.

Embodiment 79. The compound of Embodiments 77 or 78, wherein each R^{a} present on L^{A4} is independently F or C₁₋₃ alkyl optionally substituted with 1-3 F.

Embodiment 80. The compound of any one of Embodiments 49-76, wherein L^{A4} is C₃₋₁₀ cycloalkylene optionally substituted with 1-3 R^{a}.

Embodiment 81. The compound of any one of Embodiments 49-76 or 80, wherein L^{A4} is 1,4-cyclohexylene optionally substituted with 1-3 R^{a}.

Embodiment 82. The compound of any one of Embodiments 49-75, wherein L^{A4} is phenylene or 5-6 membered heteroarylene, each of which is optionally substituted with 1-3 R^{a}.

Embodiment 83. The compound of any one of Embodiments 49-75 or 82, wherein L^{A4} is:
(a) phenylene optionally substituted with 1-3 R^{a};
(b) 1,2-phenylene optionally substituted with 1-3 R^{a}; or
(c) 1,4-phenylene optionally substituted with 1-3 R^{a}.

Embodiment 84. The compound of any one of Embodiments 1-25 or 45, wherein L is:
-(L^{A3})₀₋₁-(L^{A1})₂₋₉-(L^{A3})₀₋₁-(piperazinylene)-_{bb},
wherein bb represents the point of attachment to Ring C.

Embodiment 85. The compound of Embodiment 84, wherein L is a divalent group of Formula (L-6):

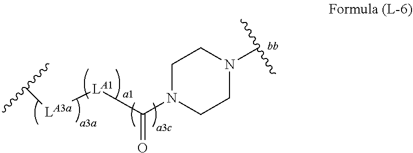

wherein:
L^{A3a} is selected from the group consisting of: —O—, —N(H)—, and —N(C₁₋₃ alkyl)-;
a3a is 0 or 1;
a1 is an integer from 2 to 11;
a3c is 0 or 1; and
bb represents the point of attachment to Ring C.

Embodiment 86. The compound of Embodiment 85, wherein a3a is 1.

Embodiment 87. The compound of Embodiments 85 or 86, wherein L^{A3}a is —O—.

Embodiment 88. The compound of any one of Embodiments 85-87, wherein 0-1 occurrence of L^{A1} is —CHR^{L}— or —C(R^{L})₂—; and each remaining occurrence of L^{A} is —CH₂—.

Embodiment 89. The compound of any one of Embodiments 1-25 or 49-52, wherein L is selected from the group consisting of:

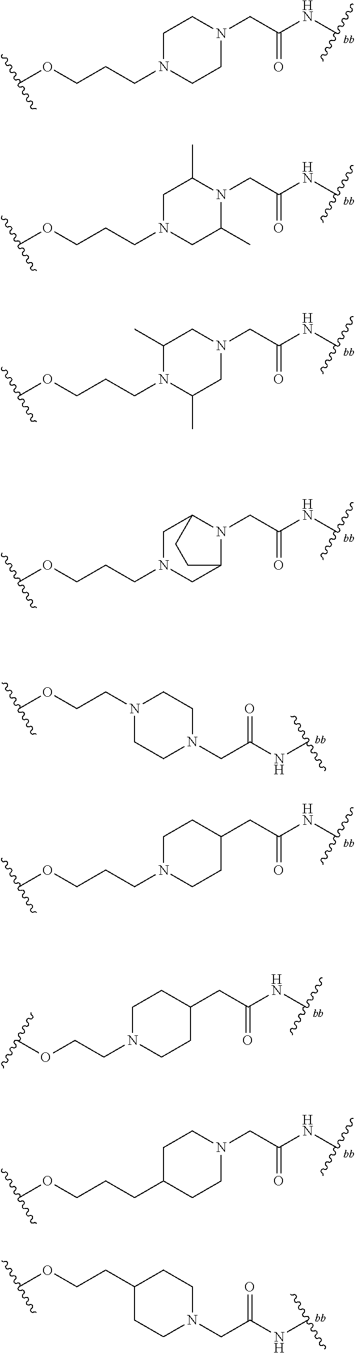

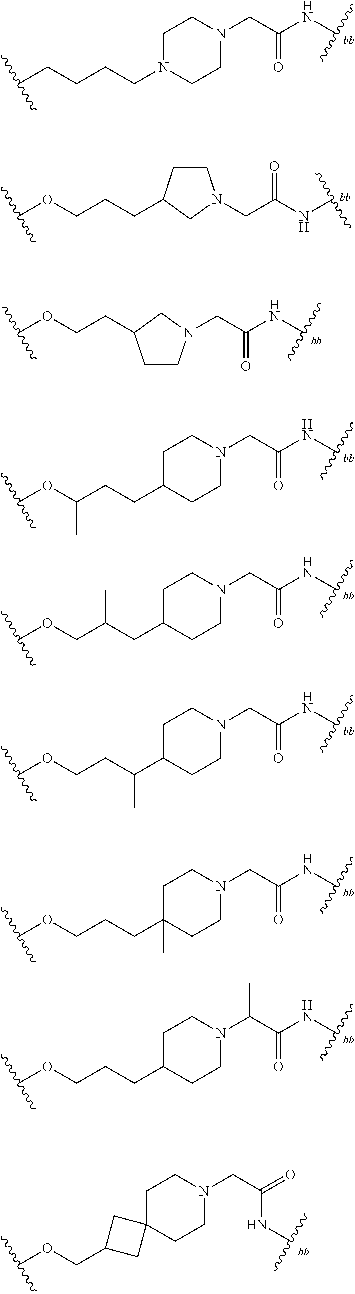

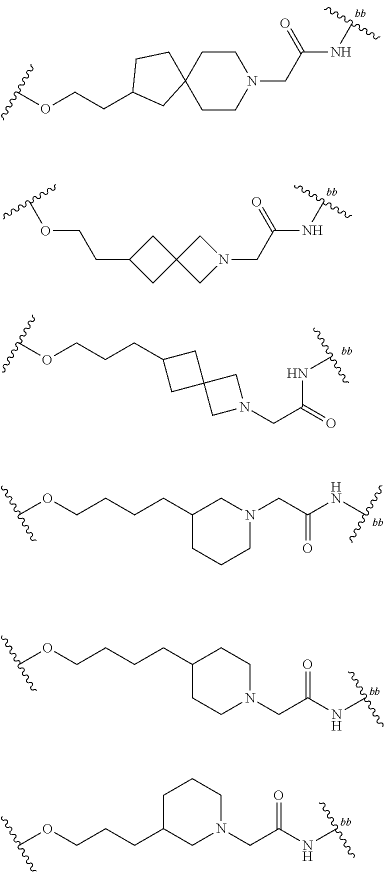

wherein bb represents the point of attachment to Ring C.

Embodiment 90. The compound of any one of Embodiments 1-25 or 49-52, wherein L is selected from the group consisting of:

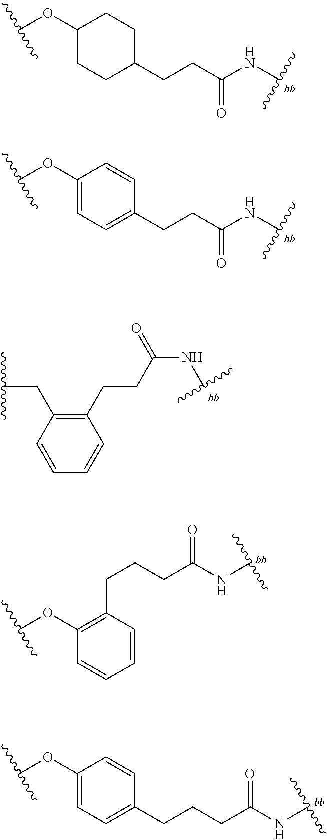

wherein bb represents the point of attachment to Ring C.

Embodiment 91. The compound of any one of Embodiments 1-25, 49, or 58-59, wherein L is

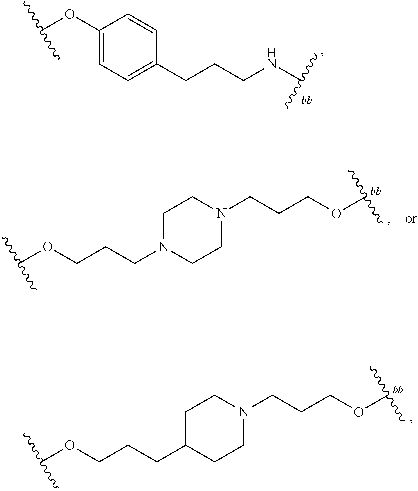

wherein bb represents the point of attachment to Ring C.

Embodiment 92. The compound of any one of Embodiments 1-25 or 66-68, wherein L is selected from the group consisting of:

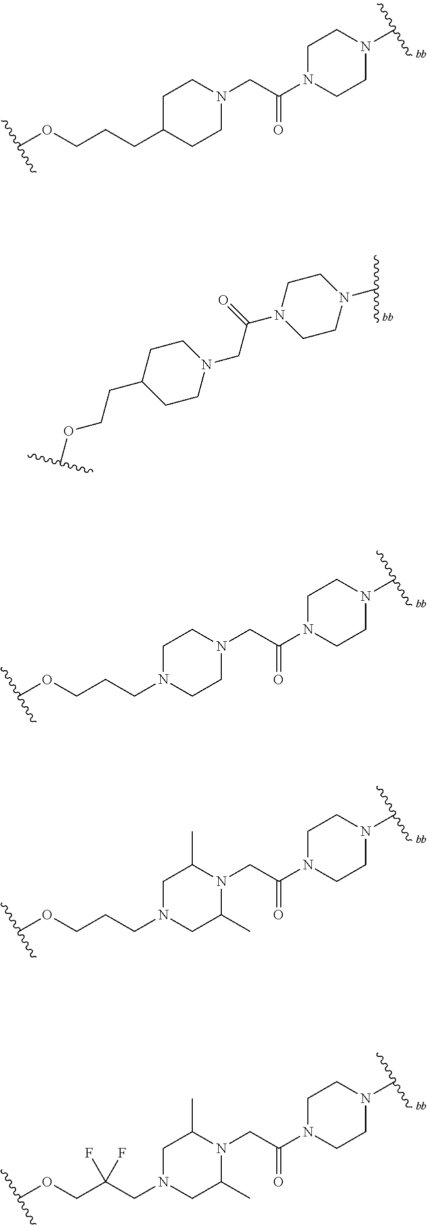

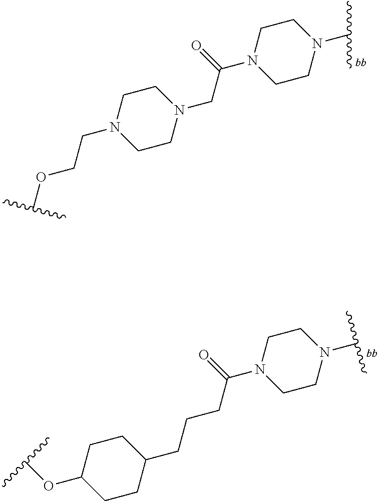

wherein bb represents the point of attachment to Ring C.

Embodiment 93. The compound of any one of Embodiments 1-25 or 66-68, wherein L is selected from the group consisting of:

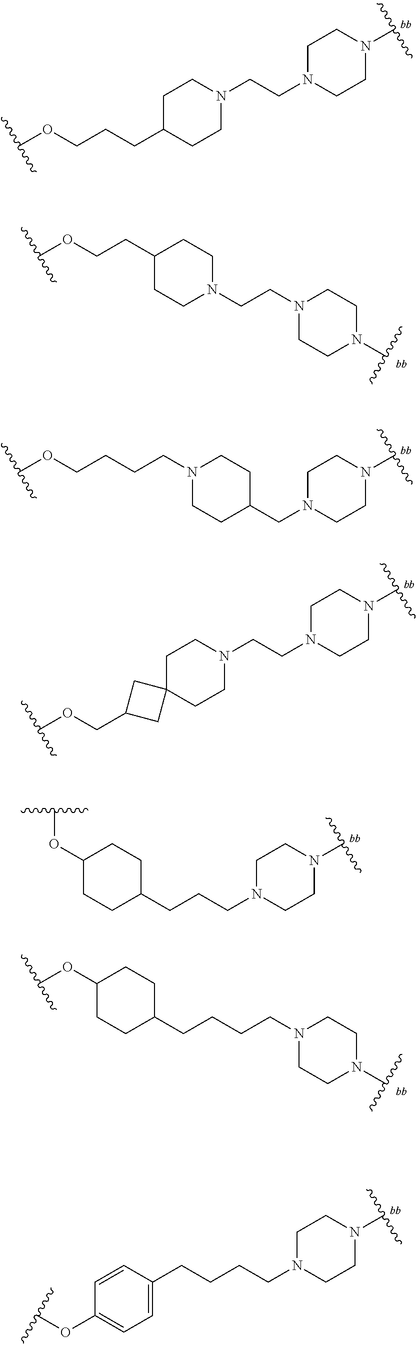

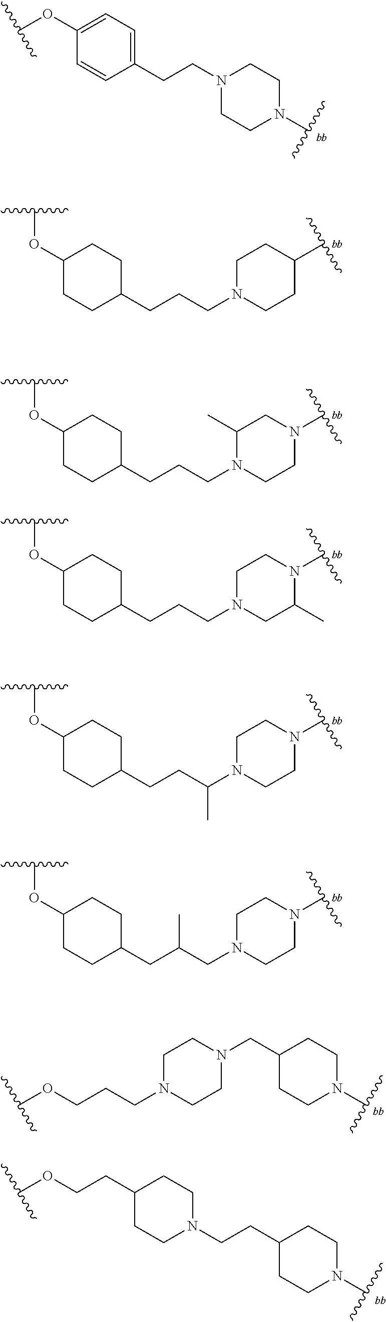

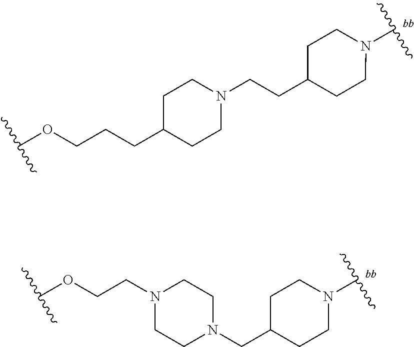

wherein bb represents the point of attachment to Ring C.

Embodiment 94. The compound of Embodiments 1 or 52, wherein the compound of Formula (I) is a compound of Formula (I-A):

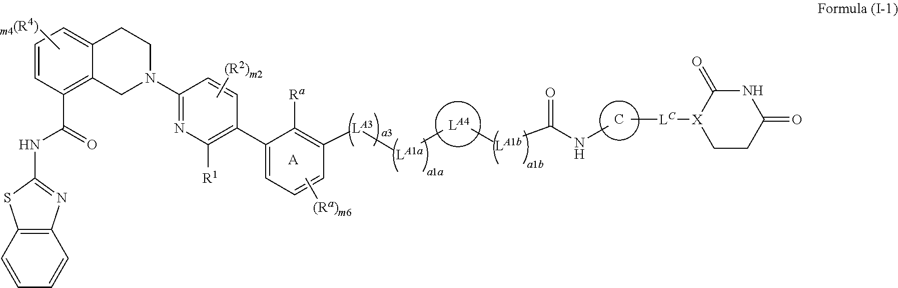

or a pharmaceutically acceptable salt thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 1 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

Embodiment 95. The compound of Embodiments 1 or 52, wherein the compound of Formula (I) is a compound of Formula (I-B):

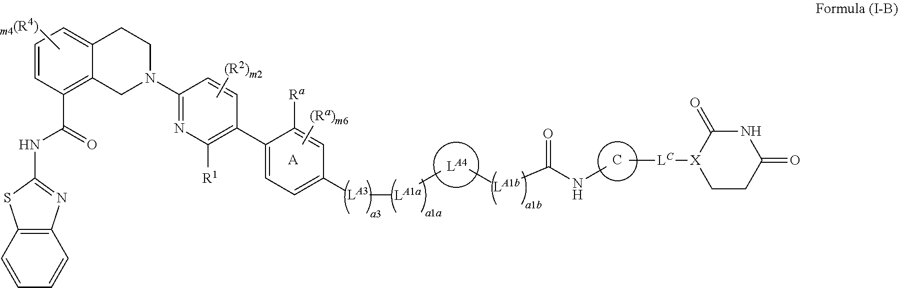

or a pharmaceutically acceptable salt thereof, wherein: PGP-1
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 1 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

Embodiment 96. The compound of Embodiments 94 or 95, wherein a3 is 1.

Embodiment 97. The compound of any one of Embodiments 94-96, wherein L^{A3} is —O—.

Embodiment 98. The compound of Embodiments 94 or 95, wherein a3 is 0.

Embodiment 99. The compound of Embodiments 1 or 59, wherein the compound of Formula (I) is a compound of Formula (I-C) or (I-D):

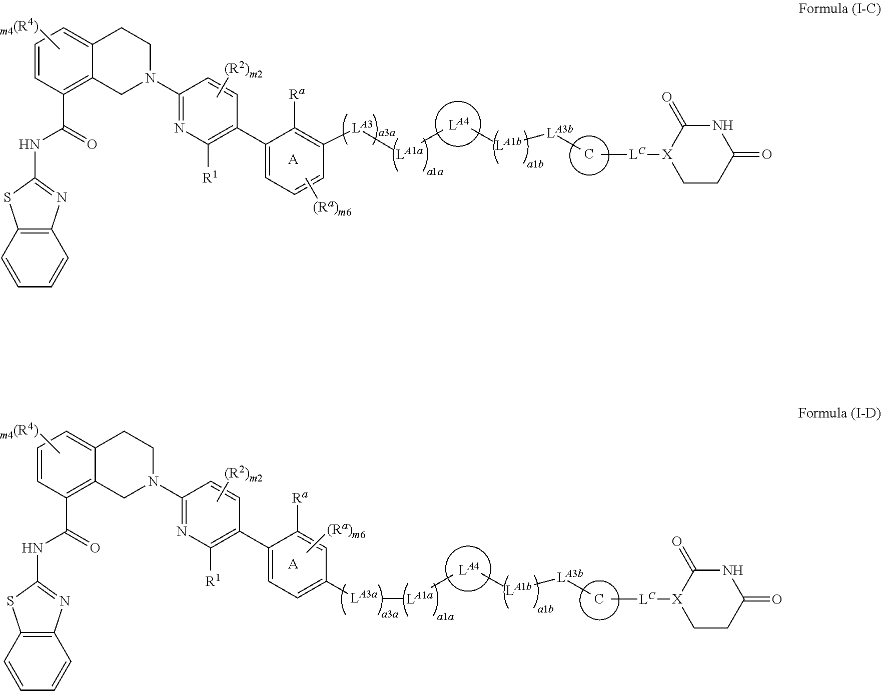

or a pharmaceutically acceptable salt thereof, wherein:
m6 is 0 or 1;
a3a is 0 or 1;
L^{A3a} and L^{A3b} are independently selected from the group consisting of: —O—, —N(H)—, and —N(C₁₋₃ alkyl)-;
a1a and a1b are independently integers from 0 to 5, provided that a1a+a1b is from 2 to 7;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

Embodiment 100. The compound of Embodiment 99, wherein the compound is a compound of Formula (I-C).

Embodiment 101. The compound Embodiments 99 or 100, wherein L^{A3b} is —N(H)— or —N(C₁₋₃ alkyl)-.

Embodiment 102. The compound of any one of Embodiments 99-101, wherein a3a is 1.

Embodiment 103. The compound of any one of Embodiments 99-102, wherein L^{A3}a is —O—.

Embodiment 104. The compound of any one of Embodiments 99-101, wherein a3a is 0.

Embodiment 105 The compound of Embodiments 1 or 68, wherein the compound of Formula (I) is a compound of Formula (I-E):

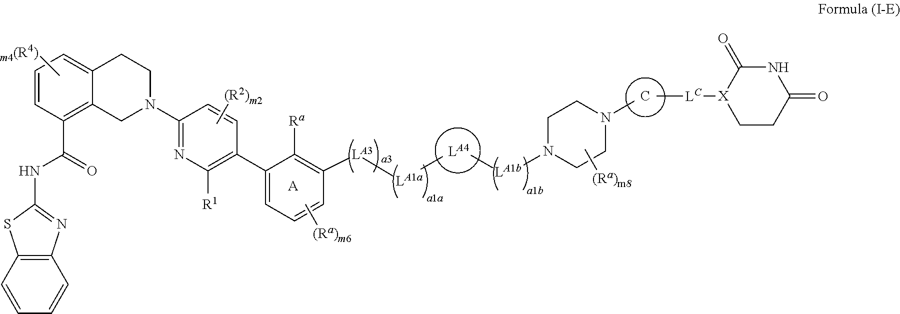

or a pharmaceutically acceptable salt thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
m8 is 0, 1, or 2.

Embodiment 106. The compound of Embodiments 1 or 68, wherein the compound of Formula (I) is a compound of Formula (I-Ea):

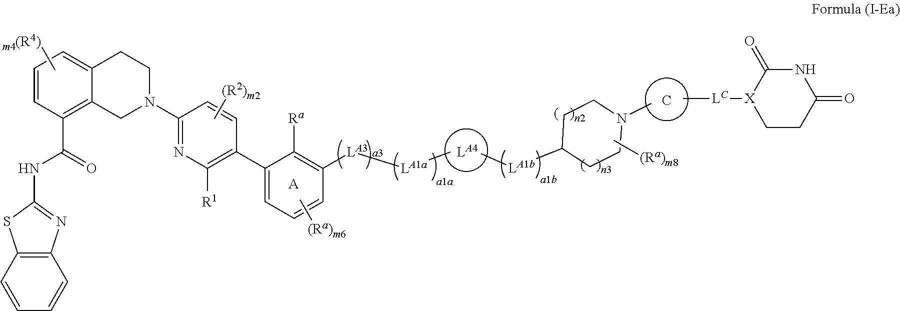

or a pharmaceutically acceptable salt thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
n2 and n3 are independently 0, 1, or 2; and
m8 is 0, 1, or 2.

Embodiment 107. The compound of Embodiments 1 or 68, wherein the compound of Formula (I) is a compound of Formula (I-Eb):

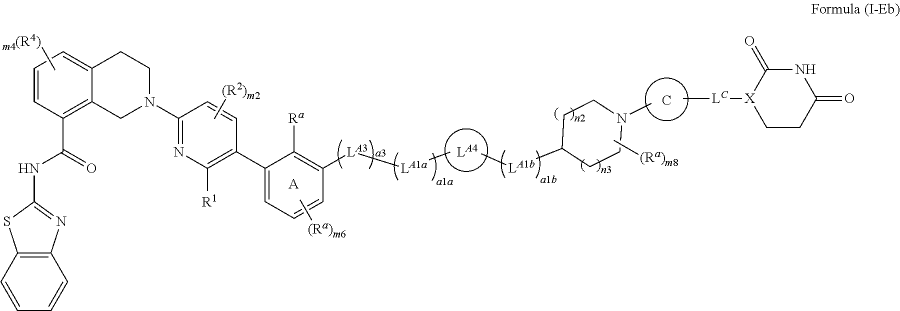

or a pharmaceutically acceptable salt thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
n2 and n3 are independently 0, 1, or 2; and
m8 is 0, 1, or 2.

Embodiment 108. The compound of Embodiments 1 or 68, wherein the compound of Formula (I) is a compound of Formula (I-F):

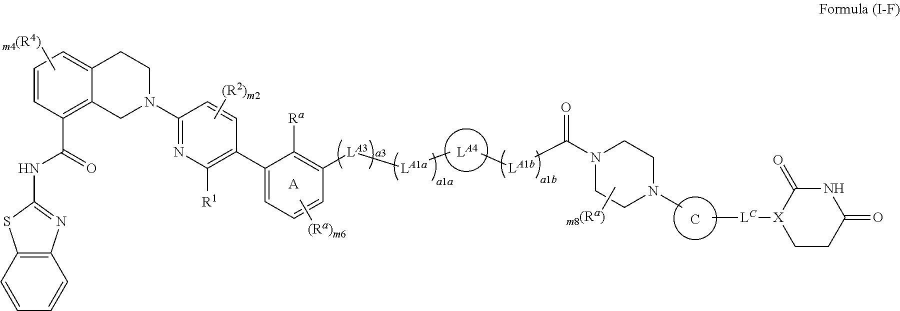

or a pharmaceutically acceptable salt thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
m8 is 0, 1, or 2.

Embodiment 109. The compound of Embodiments 1 or 68, wherein the compound of Formula (I) is a compound of Formula (I-G):

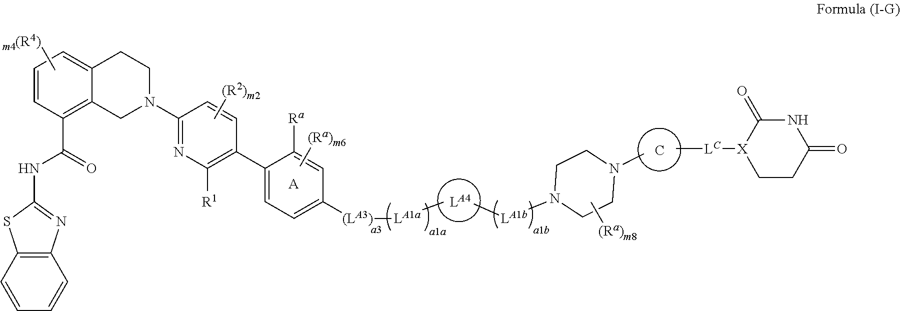

or a pharmaceutically acceptable salt thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
m8 is 0, 1, or 2.

Embodiment 110. The compound of Embodiments 1 or 68, wherein the compound of Formula (I) is a compound of Formula (I-H):

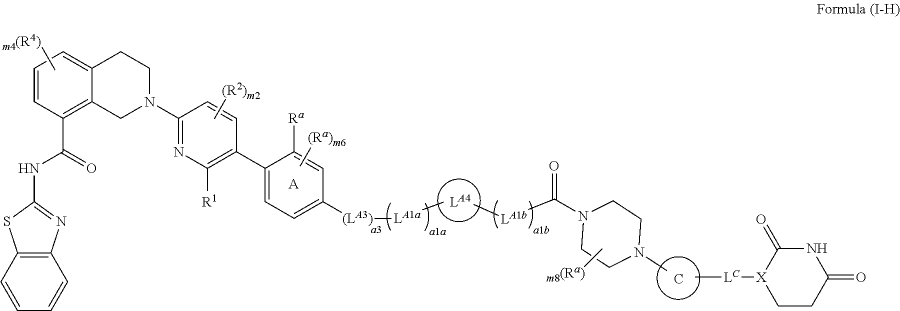

or a pharmaceutically acceptable salt thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
m8 is 0, 1, or 2.

Embodiment 111. The compound of any one of Embodiments 94-110, wherein a1a+a1b is 3.

Embodiment 112. The compound of any one of Embodiments 94-110, wherein a1a+a1b is 4.

Embodiment 113. The compound of any one of Embodiments 94-110, wherein a1a+a1b is 2 or 5.

Embodiment 114. The compound of any one of Embodiments 94-113, wherein each occurrence of L^{A1a} and L^{A1b} is —CH₂—.

Embodiment 115. The compound of any one of Embodiments 94-113, wherein one occurrence of L^{A1a} is —CHR^{L}— or —C(R^{L})₂—; each remaining occurrence of L^{A1a} is —CH₂—; and each occurrence of L^{A1b} is —CH₂—.

Embodiment 116. The compound of any one of Embodiments 94-113, wherein one occurrence of L^{A}b is —CHR^{L}— or —C(R^{L})₂—; each remaining occurrence of L^{A1b} is —CH₂—; and each occurrence of L^{A1a} is —CH₂—.

Embodiment 117. The compound of any one of Embodiments 94-116, wherein L^{A4} is 4-10 membered heterocyclylene optionally substituted with 1-3 R^{a}.

Embodiment 118. The compound of any one of Embodiments 94-117, wherein L^{A4} is selected from the group consisting of:

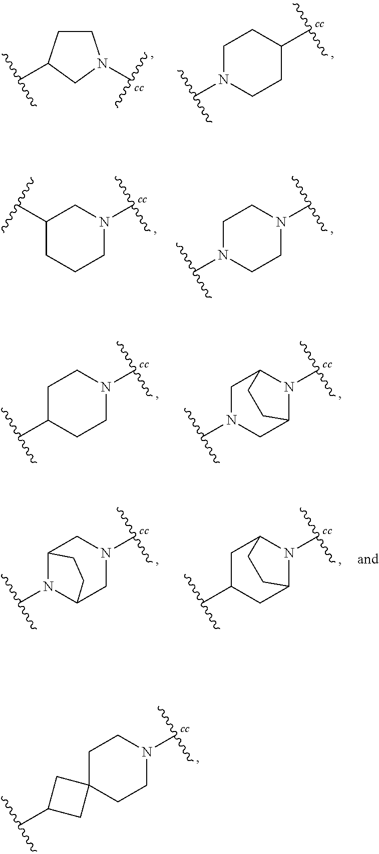

each optionally substituted with 1-3 R^{a} at one or more ring carbon atoms, wherein cc represents the point of attachment to L^{A1b}.

Embodiment 119. The compound of any one of Embodiments 94-118, wherein each R^{a} present on L^{A4} is independently a C₁-3 alkyl optionally substituted with 1-3 F.

Embodiment 120. The compound of any one of Embodiments 94-116, wherein L^{A4} is C₃₋₁₀ cycloalkylene optionally substituted with 1-3 R^{a}.

Embodiment 121. The compound of any one of Embodiments 94-116 or 120, wherein L^{A4} is 1,4-cyclohexylene optionally substituted with 1-3 R^{a}.

Embodiment 122. The compound of any one of Embodiments 94-116, wherein L^{A4} is phenylene or 5-6 membered heteroarylene, each of which is optionally substituted with 1-3 R^{a}.

Embodiment 123. The compound of any one of Embodiments 94-116 or 122, wherein L^{A4} is 1,4-phenylene optionally substituted with 1-3 R^{a}.

Embodiment 124. The compound of any one of Embodiments 94-116 or 122, wherein L^{A4} is 1,2-phenylene or 1,3-phenylene, each of which is optionally substituted with 1-3 R^{a}.

Embodiment 125. The compound of any one of Embodiments 94-124, wherein m6 is 0.

Embodiment 126. The compound of any one of Embodiments 94-125, wherein the R^{a} present on Ring A is C₁₋₃ alkyl optionally substituted with 1-3-F.

Embodiment 127. The compound of any one of Embodiments 94-126, wherein R¹ is C(O)OH.

Embodiment 128. The compound of any one of Embodiments 94-127, wherein m2 is 0.

Embodiment 129. The compound of any one of Embodiments 94-128, wherein m4 is 0.

Embodiment 130. The compound of any one of Embodiments 94-129, wherein Ring C is

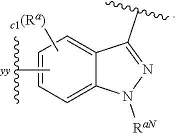

Embodiment 131. The compound of any one of Embodiments 94-130, wherein Ring C is

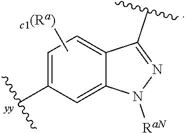

Embodiment 132. The compound of any one of Embodiments 94-130, wherein Ring C is

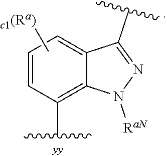

Embodiment 133. The compound of any one of Embodiments 94-132, wherein R^{aN} is C₁₋₃ alkyl.

Embodiment 134. The compound of any one of Embodiments 94-133, wherein R^{aN} is methyl.

Embodiment 135. The compound of any one of Embodiments 94-134, wherein c1 is 0.

Embodiment 136. The compound of any one of Embodiments 94-135, wherein L^{C} is a bond.

Embodiment 137. The compound of any one of Embodiments 94-136, wherein X is CH.

Embodiment 138. The compound of any one of Embodiments 94-129, wherein the

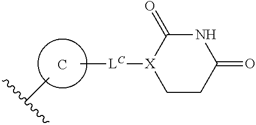

moiety is

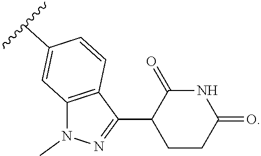 [SMILES: *c1ccc2c(C3CCC(=O)NC3=O)nn(C)c2c1]

Embodiment 139. The compound of any one of Embodiments 94-129, wherein the

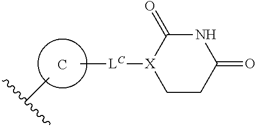

moiety is

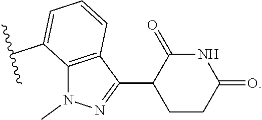 [SMILES: *c1cccc2c(C3CCC(=O)NC3=O)nn(C)c12]

Embodiment 140. A compound selected from the group consisting of 101, 102, 103, 104, 105, 106, 107, 108, 109, 110, 111, 112, 113, 114, 115, 116, 116a, 117, 117a, 118, 119, 120, 121, 122, 123, 124, 124a, 125, 125a, 126, 127, 128, 129, 130, 131, 131a, 132, 133, 134, 135, 136, 137, 138, 138a, 138b, 139, 140, 141, 142, 143, 144, 145, 146, 146a, 146b, 147, 147a, 147b, 148, 148a, 148b, 149, 150, 151, 152, 153, 154, 154a, 155, 156, 157, 158, 159, 159a, 160, 160a, 160b, 161, 161a, 162, 162a, 162b, 163, 164, 164a, 164b, 165, 166, 167, 167a, 167b, 168, 168a, 169, 169a, 169b, 170, 171, 171a, 172, 173, 173a, 173b, 174, 174a, 174b, 175, 176, 176a, 177, 177a, 178, 179, 179a, 180, 180a, 180b, 181, 182, 182a, 183, 183a, 184, 185, 185a, 186, 186a, 187, 187a, 187b, 188, 189, 190, 191, 192, 193, 193a, 193b, 194, 195, 196, 197, 197a, 197b, 198, 199, 200, 201, 202, 202a, 202b, 203, 204, 204a, 205, 206, 207, 207a, 207b, 208, 209, 209a, 210, 210a, 211, 211a, 212, 212a, 213, 214, 214a, 215, 215a, 216, 216a, 216b, 217, 218, 218a, 219, 219a, 219b, 220, 221, 222, 223, 224, 224a, 224b, 225, 226, 227, 227a, 228, 229, 230, 231, 231a, 231b, 232, 233, 234, 235, 236, 237, 238, 239, 239a, 240, 240a, 240b, 241, 242, 243, 244, 245, 245a, 245b, 246, 246a, 246b, 247, 248, 249, 250, 250a, 251, 251a, 252, 252a, 253, 253a, 254, 254a, 255, 256, 257, 258, 259, 259a, 260, 261, 262, 262a, 262b, 263, 263a, 263b, 264, 265, 266, 266a, 266b, 267, 268, 268a, 268b, 269, 269a, 270, 270a, 271, 271a, 271b, 272, 272a, 272b, 273, 273a, 273b, 274, 274a, 274b, 275, 275a, 276, 276a, 276b, 277, 278, 278a, 278b, 279, 280, 281, 281a, 281b, 282, 282a, 282b, 283, 283a, 283b, 284, 284a, 284b, 285, 286, 287, 288, 289, 289a, 289b, 290, 291, 292, 293, 293a, 294, 295, 296, 296a, 297, 297a, 297b, 298, 298a, 299, 299a, 300, 300a, 300b, 301, 301a, 301b, 302, 303, 304, 305, 306, 307, 307a, 307b, 308, 308a, 308b, 309, 310, 310a, 310b, 311, 311a, 312, 313, 314, 316, 316a, 317, 318, 318a, 319, 319a, 320, 320a, 320b, 321, 321a, 321b, 322, 322a, 323, 323a, 324, 324a, 325, 325a, 326, 326a, 327, 327a, 328, 328a, 329, 329a, 330, 330a, 331, 331a, 331b, 332, 332a, 332b, 333, 333a, 334, 334a, 335, 335a, 336, 336a, 337, 337a, 338, 338a, 339, 339a, 340, 340a, 341, 341a, 342, 342a, 343, 343a, 343b, 343c, 343d, 344, 344a, 344b, 344c, 344d, 345, 345a, 346, 346a, 346b, 347, 347a, 347b, 349, 349a, 349b, 350, 350a, 351, 351a, 351b, 352, 352a, and 352b as depicted in Table C1, or a pharmaceutically acceptable salt thereof, provided that the compound does not exhibit Yₘᵢₙ (6 h)≥70% and/or Yₘᵢₙ (24 h)≥70% under conditions described in Example B1.

Embodiment 141. A pharmaceutical composition comprising a compound of any one of Embodiments 1-140, or a pharmaceutically acceptable salt thereof, and a pharmaceutically acceptable excipient.

Embodiment 142. A BCL-X_{L} protein non-covalently bound with a compound of any one of Embodiments 1-140, or a pharmaceutically acceptable salt thereof.

Embodiment 143. A ternary complex comprising a BCL-X_{L} protein, a compound of any one of Embodiments 1-140, or a pharmaceutically acceptable salt thereof, and a CRBN protein.

Embodiment 144. A method for treating a cancer in a subject in need thereof, the method comprising administering to the subject a therapeutically effective amount of a compound of any one of Embodiments 1-140, or a pharmaceutically acceptable salt thereof, or a pharmaceutical composition according to Embodiment 141.

Embodiment 145. The method of Embodiment 144, further comprising administering an additional therapy or therapeutic agent.

Embodiment 146. The method of Embodiment 145, wherein the additional therapy or therapeutic agent is an ALK inhibitor, a BCL-2 inhibitor, a BCR-Abl inhibitor, a BRaf inhibitor, a CDK2 inhibitor, CDK 4/6 inhibitor, a CDK7 inhibitor, a CDK9 inhibitor, an EGFR inhibitor, an anti-EGFR antibody, an ERK inhibitor, a FGFR1 inhibitor, a FGFR2 inhibitor, a FGFR3 inhibitor, a FGFR4 inhibitor, a HER2 inhibitor, a JAK2 inhibitor, a KRas inhibitor, a MEK inhibitor, a MET inhibitor, a PARP inhibitor, an LSD1 inhibitor, a BET inhibitor, a telomerase inhibitor, a TORC1/2 inhibitor, chemotherapy, radiotherapy, or a combination thereof.

Embodiment 147. The method of Embodiment 146, wherein the additional therapy or therapeutic agent is a JAK2 inhibitor.

Embodiment 148. The method of Embodiment 147, wherein the JAK2 inhibitor is fedratinib (e.g., fedratinib dihydrochloride monohydrate), momelotinib (e.g., momelotinib dihydrochloride), pacritinib (e.g., pacritinib citrate), ruxolitinib (e.g., ruxolitinib phosphate), or a combination thereof.

Embodiment 149. The method of any one of Embodiments 144-148, wherein the cancer is breast cancer, colorectal cancer, bile duct cancer, colorectal cancer, gastrointestinal stromal tumor, pancreatic cancer, bladder cancer, kidney cancer, cervical cancer, ovarian cancer, uterine cancer, head and neck cancer, hematological cancer, lung cancer, skin cancer, or a combination thereof.

Embodiment 150. The method of Embodiment 149, wherein the hematological cancer is acute lymphocytic leukemia (ALL), acute myeloid leukemia (AML), chronic lymphocytic leukemia (CLL), follicular lymphoma (FL), small lymphocytic lymphoma (SLL), essential thrombocythemia, polycythemia vera, myelofibrosis, or a combination thereof.

Embodiment 151. The method of Embodiment 150, wherein the hematological cancer is essential thrombocythemia, polycythemia vera, myelofibrosis, or a combination thereof.

Embodiment 152. The method of Embodiment 151, wherein the myelofibrosis is primary myelofibrosis.

Embodiment 153. The method of Embodiment 151, wherein the myelofibrosis is post-essential thrombocythemia myelofibrosis.

Embodiment 154. The method of Embodiment 151, wherein the myelofibrosis is post-polycythemia vera myelofibrosis.

Embodiment 155. The method of any one of Embodiments 151-154, wherein the hematological cancer has a JAK2 mutation (e.g., JAK2 V617F).

Embodiment 156. The method of Embodiment 150, wherein the ALL is T-ALL.

Embodiment 157. The method of Embodiment 150, wherein the AML is M6-AML.

Embodiment 158. The method of Embodiment 150, wherein the AML is M7-AML.

Embodiment 159. A method for inducing degradation of a BCL-X_{L} protein in a mammalian cell, the method comprising contacting the mammalian cell with an effective amount of a compound of any one of Embodiments 1-140, or a pharmaceutically acceptable salt thereof.

Embodiment 160. The method of Embodiment 159, wherein the contacting occurs in vivo.

Embodiment 161. The method of Embodiment 159, wherein the contacting occurs in vitro.

Embodiment 162. The method of any one of Embodiments 159-161, wherein the mammalian cell is a mammalian cancer cell.

### Exemplary Formula (I-A) Embodiments

Embodiment 1. A compound of Formula (I-A):

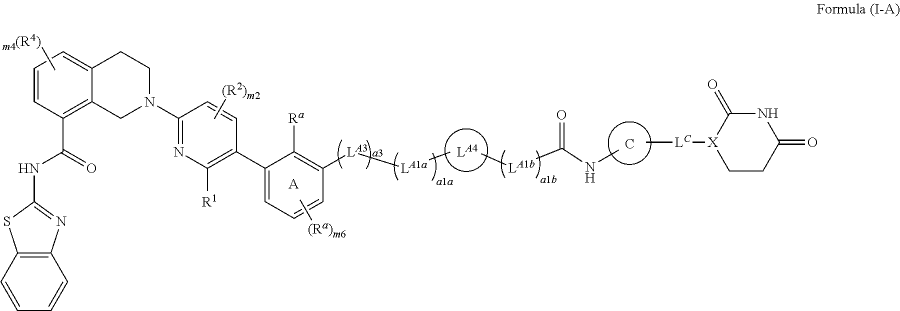

or a pharmaceutically acceptable salt thereof, wherein:
R¹ is selected from the group consisting of:
(a) C(O)OH,
(b) C(O)NR^{d}R^{e}, and
(c) C(O)OC₁₋₆ alkyl, wherein the C₁₋₆ alkyl is optionally substituted with 1-3 R^{c};
each R² and R⁴ is independently selected from the group consisting of: halo, CN, C₁₋₃ alkyl, C₁₋₃ haloalkyl, C₁₋₃ alkoxy, C₁₋₃ haloalkoxy, OH, and NR^{d}R^{e};
m2 is 0, 1, or 2;
m4 is 0, 1, 2, or 3;
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 1 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
wherein each R^{L} is independently selected from the group consisting of: halo; cyano; —OH; —C₁₋₆ alkoxy; —C₁₋₆ haloalkoxy; —NR^{d}R^{e}; C(═O)N(R^{f})₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{c};
Ring C is selected from the group consisting of: R^{aN} and Y.

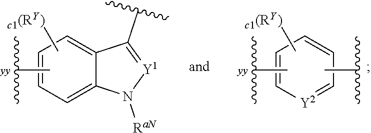

c1 is 0, 1, 2, or 3;
each R^{Y} is independently selected from the group consisting of R^{a} and R^{b};
R^{aN} is H or C₁₋₆ alkyl optionally substituted with 1-3 R^{c};
Y¹ and Y² are independently N, CH, or CR^{Y};
yy represents the point of attachment to L;
X is CH, C, or N;
the [symbol] is a single bond or a double bond;
L^{C} is selected from the group consisting of: a bond, —CH₂—, —CHR^{a}—, —C(R^{a})₂—, —N(R^{d})—, and O, provided that when X is N, then L^{C} is other than O;
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—; —N(H)—; —N(C₁₋₃ alkyl)-; —S(O)₀₋₂—; C(═O); and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R)₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(R^{f})₂; S(O)₁₋₂(C₁₋₆ alkyl); S(O)₁₋₂(C₁₋₆ haloalkyl); S(O)₁₋₂N(R)₂; and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂.

Embodiment 2. The compound of Embodiment 1, wherein a1a+a1b is 2 to 5.

Embodiment 3. The compound of Embodiment 1 or 2, wherein a1a+a1b is 3.

Embodiment 4. The compound of Embodiment 1 or 2, wherein a1a+a1b is 4.

Embodiment 5. The compound of Embodiment 1 or 2, wherein a1a+a1b is 2 or 5.

Embodiment 6. The compound of any one of Embodiments 1-5, wherein a3 is 1.

Embodiment 7. The compound of any one of Embodiments 1-6, wherein L^{A3} is —O—.

Embodiment 8. The compound of any one of Embodiments 1-5, wherein a3 is 0.

Embodiment 9. The compound of any one of Embodiments 1-8, wherein each occurrence of L^{A1a} and L^{A1b} is —CH₂—.

Embodiment 10. The compound of any one of Embodiments 1-8, wherein one occurrence of L^{A1a} is —CHR^{L}— or —C(R^{L})₂—; each remaining occurrence of L^{A1a} is —CH₂—; and each occurrence of L^{A1b} is —CH₂—.

Embodiment 11. The compound of any one of Embodiments 1-8, wherein one occurrence of L^{A1b} is —CHR^{L}— or —C(R^{L})₂—; each remaining occurrence of L^{A1b} is —CH₂—; and each occurrence of L^{A1a} is —CH₂—.

Embodiment 12. The compound of any one of Embodiments 1-11, wherein L^{A4} is 4-10 membered heterocyclylene optionally substituted with 1-3 R^{a}.

Embodiment 13. The compound of any one of Embodiments 1-12, wherein L^{A4} is selected from the group consisting of:

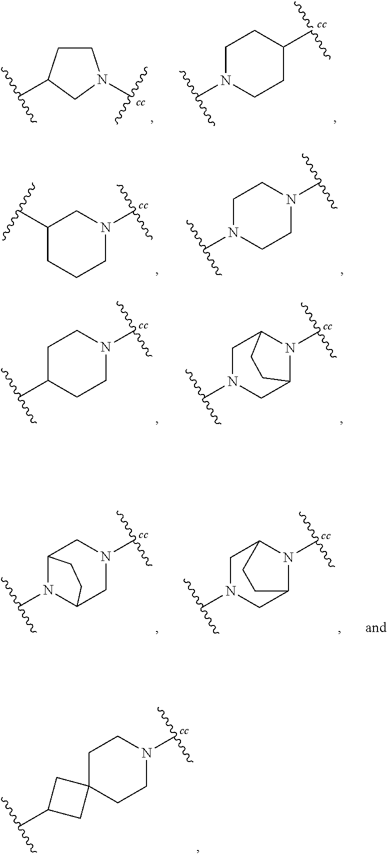

each optionally substituted with 1-3 R^{a} at one or more ring carbon atoms, wherein cc represents the point of attachment to L^{A1b}; or
wherein L^{A4} is selected from the group consisting of:

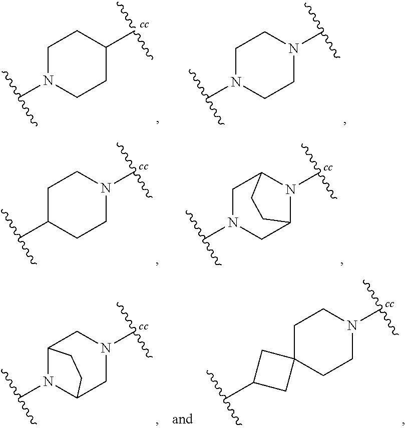 [SMILES: *C1CCN(*)CC1 ; *N1CCN(*)CC1 ; *C1CCN(*)CC1 ; *N1CC2CCC(C1)N2* ; *N1CC2CCC(C1)N2* ; *C1CC2(CCN(*)CC2)C1]

each optionally substituted with 1-3 R^{a} at one or more ring carbon atoms, wherein cc represents the point of attachment to L^{A1b}.

Embodiment 14. The compound of Embodiments 12 or 13, wherein each R^{a} present on L^{A4} is independently a C₁₋₃ alkyl optionally substituted with 1-3 F.

Embodiment 15. The compound of any one of Embodiments 1-11, wherein L^{A4} is C₃₋₁₀ cycloalkylene optionally substituted with 1-3 R^{a}.

Embodiment 16. The compound of any one of Embodiments 1-11 or 15, wherein L^{A4} is 1,4-cyclohexylene optionally substituted with 1-3 R^{a}.

Embodiment 17. The compound of any one of Embodiments 1-11, wherein L^{A4} is phenylene or 5-6 membered heteroarylene, each of which is optionally substituted with 1-3 R^{a}.

Embodiment 18. The compound of any one of Embodiments 1-11 or 17, wherein L^{A4} is 1,4-phenylene optionally substituted with 1-3 R^{a}.

Embodiment 19. The compound of any one of Embodiments 1-11 or 17, wherein L^{A4} is 1,2-phenylene or 1,3-phenylene, each of which is optionally substituted with 1-3 R^{a}.

Embodiment 20. The compound of any one of Embodiments 1-19, wherein m6 is 0.

Embodiment 21. The compound of any one of Embodiments 1-20, wherein the R^{a} present on Ring A is C₁₋₃ alkyl optionally substituted with 1-3-F.

Embodiment 22. The compound of any one of Embodiments 1-21, wherein R¹ is C(O)OH.

Embodiment 23. The compound of any one of Embodiments 1-22, wherein m2 is 0.

Embodiment 24. The compound of any one of Embodiments 1-23, wherein m4 is 0.

Embodiment 25. The compound of any one of Embodiments 1-24, wherein Ring C is

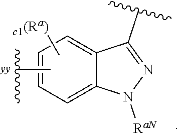

Embodiment 26. The compound of any one of Embodiments 1-25, wherein Ring C is

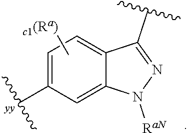

Embodiment 27. The compound of any one of Embodiments 1-25, wherein Ring C is

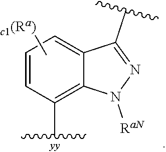

Embodiment 28. The compound of any one of Embodiments 1-27, wherein R^{aN} is C₁₋₃ alkyl.

Embodiment 29. The compound of any one of Embodiments 1-28, wherein R^{aN} is methyl.

Embodiment 30. The compound of any one of Embodiments 1-29, wherein c1 is 0.

Embodiment 31. The compound of any one of Embodiments 1-30, wherein L^{C} is a bond.

Embodiment 32. The compound of any one of Embodiments 1-31, wherein X is CH.

Embodiment 33. The compound of any one of Embodiments 1-24, wherein the

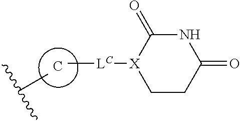

moiety is

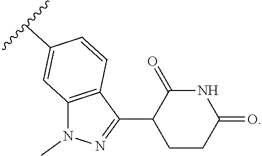 [SMILES: *c1ccc2c(C3CCC(=O)NC3=O)nn(C)c2c1]

Embodiment 34. The compound of any one of Embodiments 1-24, wherein the

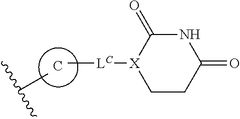

moiety is

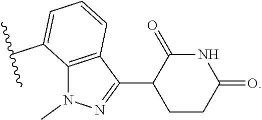 [SMILES: *c1cccc2c(C3CCC(=O)NC3=O)nn(C)c12]

Embodiment 35. The compound of Embodiment 1, wherein the compound of Formula (I-A) is a compound of Formula (I-A-1):

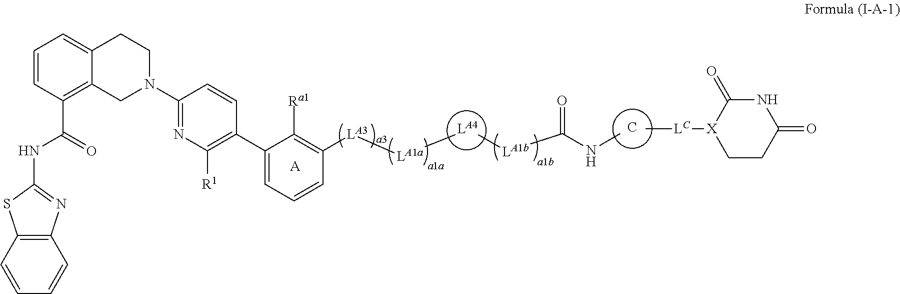

or a pharmaceutically acceptable salt thereof, wherein:
R¹ is C(O)OH or C(O)OC₁₋₆ alkyl;
R^{a1} is C₁₋₃ alkyl optionally substituted with 1-3 F;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 1 to 5;
L^{A1a} and L^{A1b} are independently selected from the group consisting of: —CH₂—, —CHR^{L}—, and —C(R^{L})₂—; and
L^{A4} is selected from the group consisting of:
c) C₃₋₁₀ cycloalkylene or 4-10 membered heterocyclylene, each of which is optionally substituted with 1-3 R^{a}; and
d) phenylene or 5-6 membered heteroarylene, each of which is optionally substituted with 1-3 R^{a}.

Embodiment 36. The compound of Embodiment 1, wherein the compound of Formula (I-A) is a compound of Formula (I-A-2):

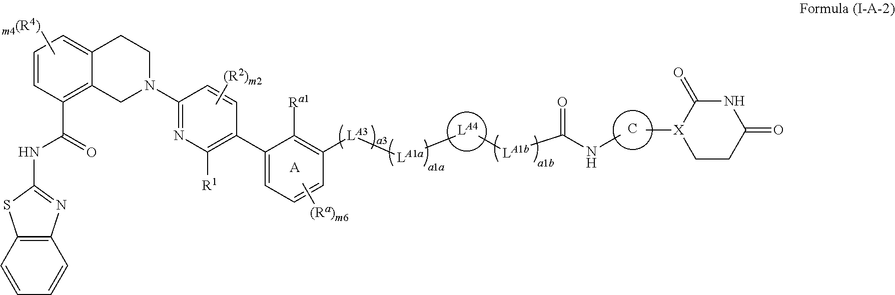

or a pharmaceutically acceptable salt thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 1 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and

### Ring C is

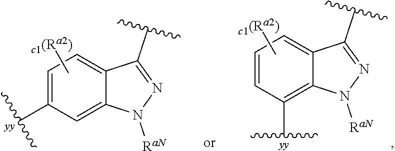

wherein: c1 is 0 or 1; R^{a2} is selected from the group consisting of: halo (e.g., —F) and C₁₋₃ alkyl optionally substituted with 1-3 F; and R^{aN} is C₁₋₃ alkyl.

Embodiment 37. The compound of any one of Embodiments 1, 35 or 36, wherein the compound of Formula (I-A), Formula (I-A-1), or Formula (I-A-2) is a compound is a compound of Formula (I-A-3):

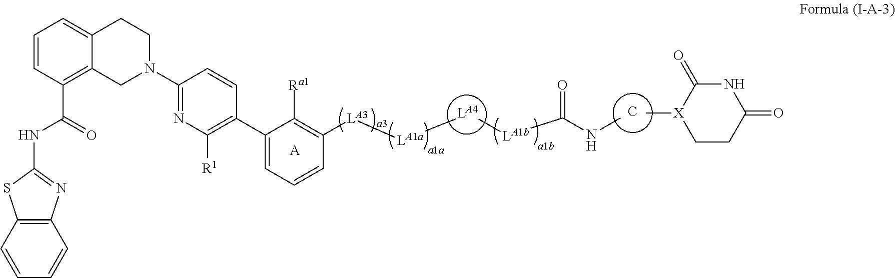

or a pharmaceutically acceptable salt thereof, wherein:
R¹ is C(O)OH or C(O)OC₁₋₆ alkyl;
R^{a1} is C₁₋₃ alkyl optionally substituted with 1-3 F;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 1 to 5 (e.g., from 2 to 5);
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and

### Ring C is

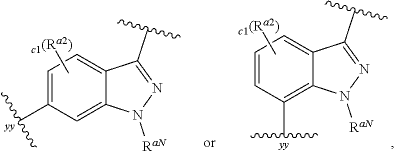

wherein: c1 is 0 or 1; R^{a2} is selected from the group consisting of: halo (e.g., —F) and C₁₋₃ alkyl optionally substituted with 1-3 F; and R^{aN} is C₁₋₃ alkyl.

Embodiment 38. The compound of any one of Embodiments 36 or 37, wherein R¹ is C(O)OH.

Embodiment 39. The compound of any one of Embodiments 35-38, wherein c1 is 0.

Embodiment 40. The compound of any one of Embodiments 35-39, wherein a1a+a1b is from 2 to 5.

Embodiment 41. The compound of any one of Embodiments 1 or 35-40, wherein the

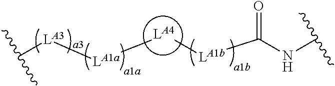

moiety is selected from the groups depicted in Table (L-I-A):

TABLE (L-I-A)

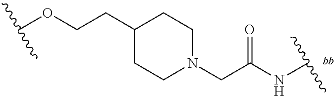

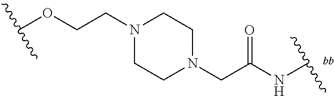

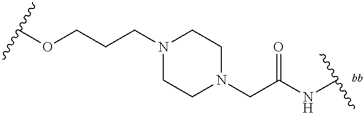

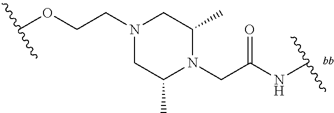

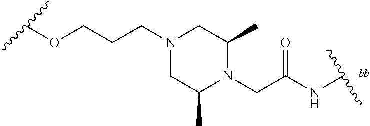

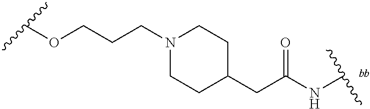

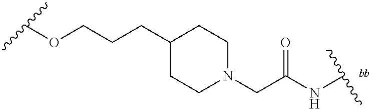

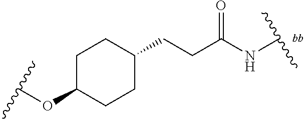

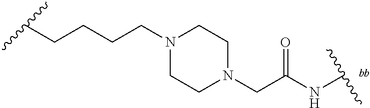

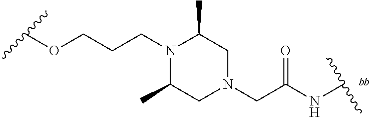

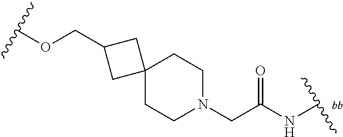

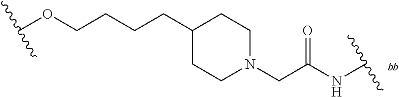

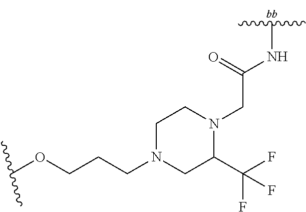

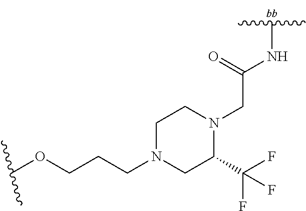

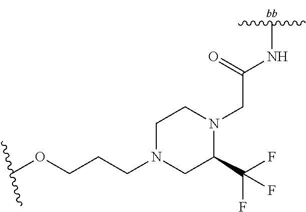

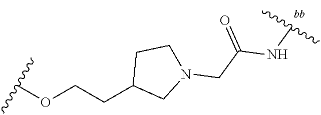

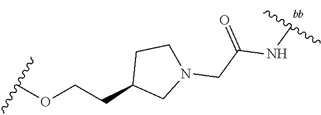

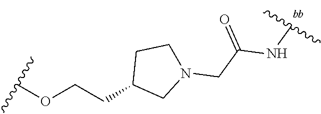

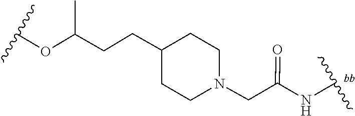

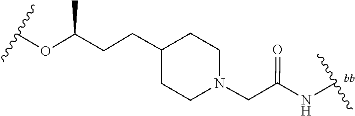

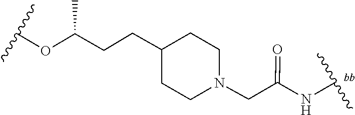

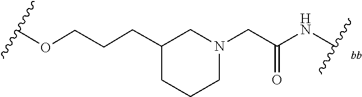

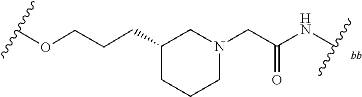

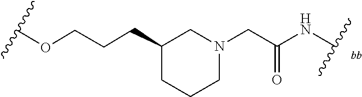

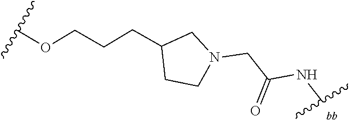

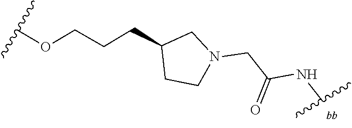

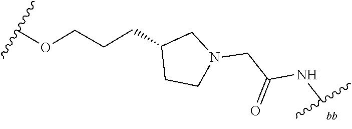

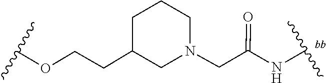

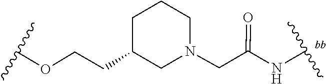

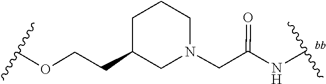

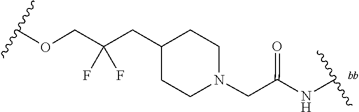

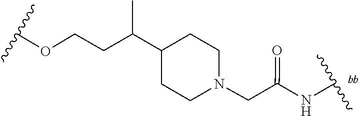

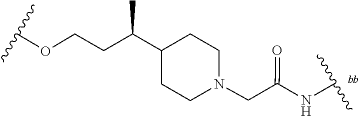

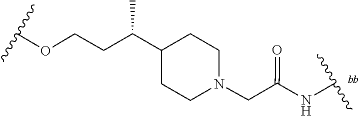

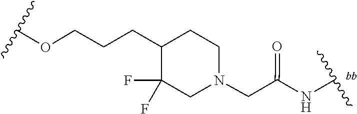

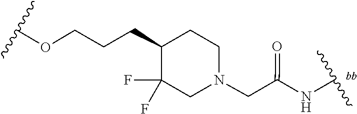

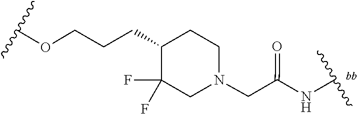

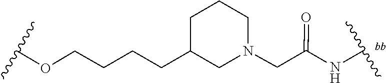

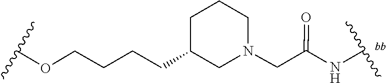

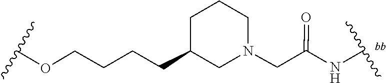

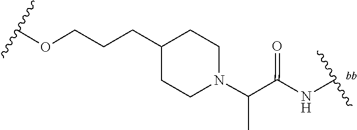

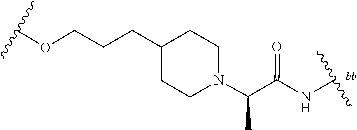

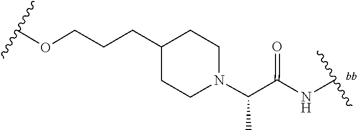

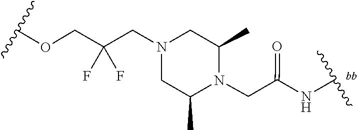

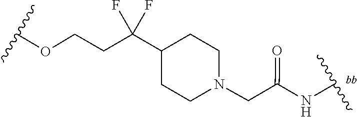

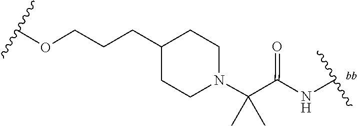

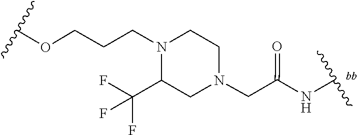

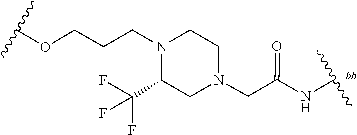

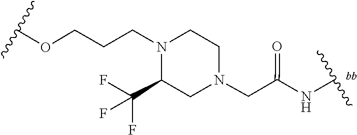

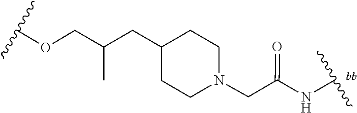

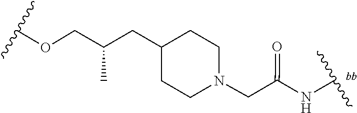

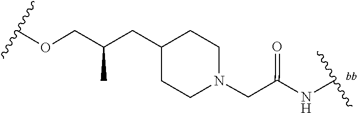

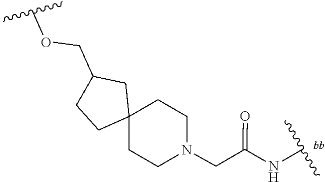

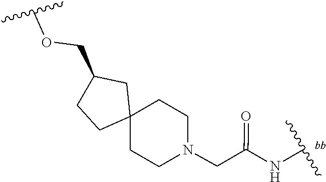

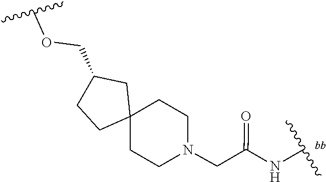

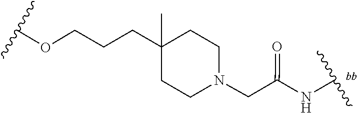

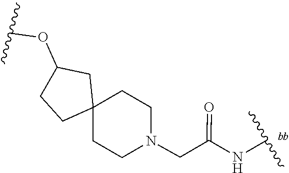

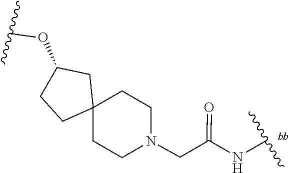

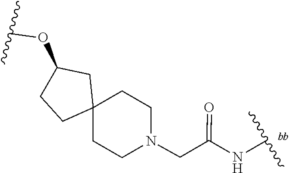

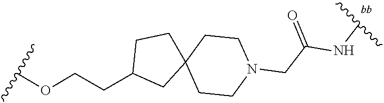

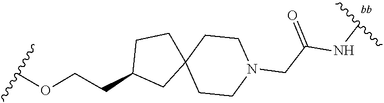

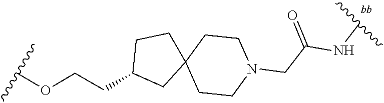

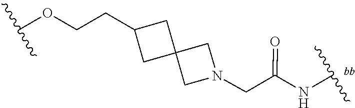

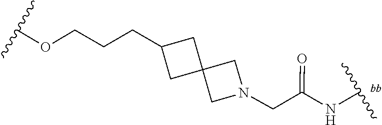

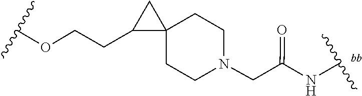

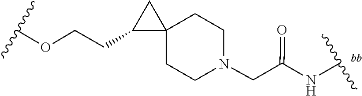

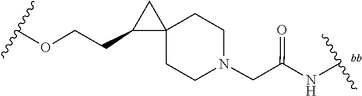

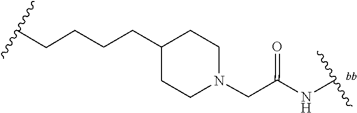

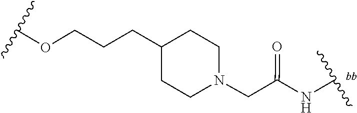

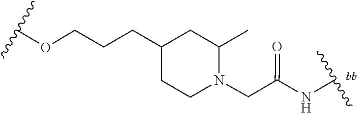

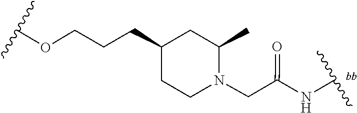

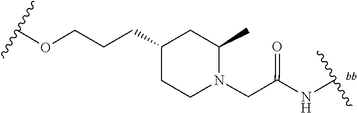

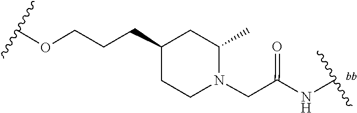

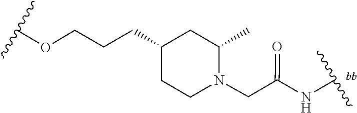

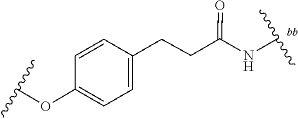

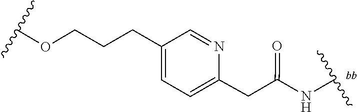

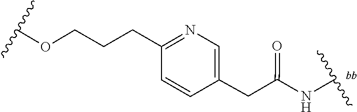

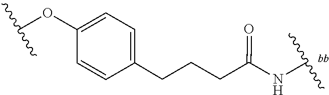

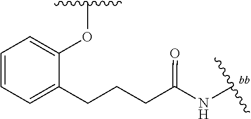

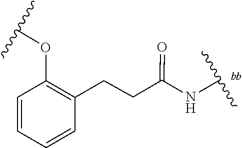

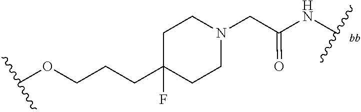

wherein bb represents the point of attachment to Ring C; or
wherein the

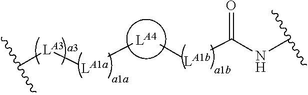

moiety is selected from the group consisting of:

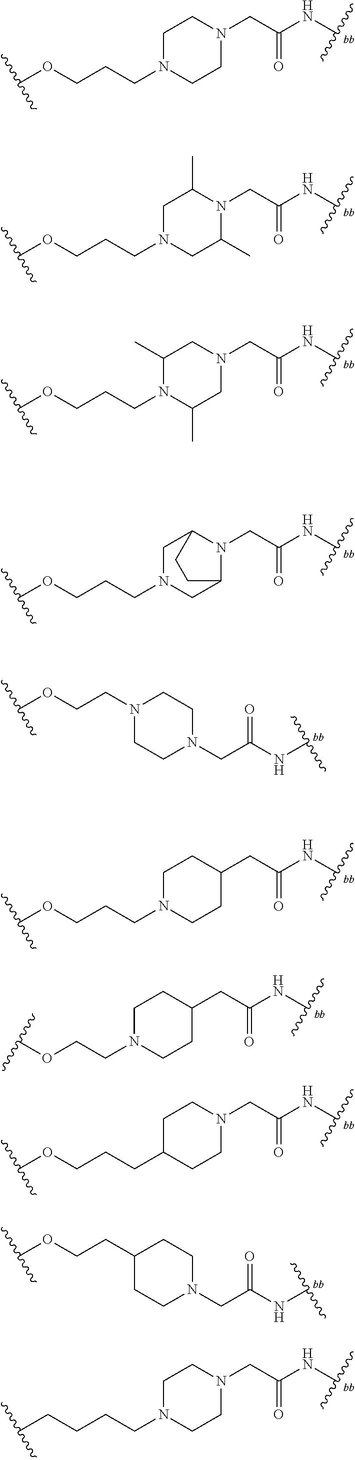

wherein bb represents the point of attachment to Ring C.

Embodiment 42. The compound of any one of Embodiments 1-41, wherein the compound is selected from the group consisting of 101, 102, 103, 104, 105, 106, 107, 116, 116a, 117, 117a, 120, 121, 122, 124, 124a, 125, 125a, 126, 127, 128, 129, 130, 131, 131a, 132, 134, 156, 158, 159, 159a, 160, 160a, 160b, 162, 162a, 162b, 164, 164a, 164b, 166, 167, 167a, 167b, 169, 169a, 169b, 170, 173, 173a, 173b, 177, 177a, 180, 180a, 180b, 181, 186, 186a, 186b, 187, 187a, 187b, 193, 193a, 193b, 196, 197, 197a, 197b, 202, 202a, 202b, 203, 207, 207a, 207b, 209, 209a, 210, 210a, 210b, 213, 216, 216a, 216b, 218, 218a, 218b, 219, 219a, 219b, 226, 229, 231, 231a, 231b, 240, 240a, 240b, 243, 245, 245a, 245b, 246, 246a, 246b, 262, 262a, 262b, 263, 263a, 263b, 267, 268, 268a, 268b, 272, 272a, 272b, 277, 280, 286, 289, 289a, 289b, 292, 295, 302, 309, 313, 317, 343, 343a, 343b, 343c, 343d, and 357, as depicted in Table C1, or pharmaceutically acceptable salts thereof, or
the compound is selected from the group consisting of 101, 102, 103, 104, 105, 106, 107, 116, 116a, 117, 117a, 120, 121, 122, 124, 124a, 125, 125a, 126, 127, 128, 129, 130, 131, 131a, 132, 134, 156, 158, 159, 159a, 160, 160a, 160b, 162, 162a, 162b, 164, 164a, 164b, 166, 167, 167a, 167b, 169, 169a, 169b, 170, 173, 173a, 173b, 177, 177a, 180, 180a, 180b, 181, 186, 186a, 187, 187a, 187b, 193, 193a, 193b, 196, 197, 197a, 197b, 202, 202a, 202b, 203, 207, 207a, 207b, 209, 209a, 210, 210a, 213, 216, 216a, 216b, 218, 218a, 219, 219a, 219b, 226, 229, 231, 231a, 231b, 240, 240a, 240b, 243, 245, 245a, 245b, 246, 246a, 246b, 262, 262a, 262b, 263, 263a, 263b, 267, 268, 268a, 268b, 272, 272a, 272b, 277, 280, 286, 289, 289a, 289b, 292, 295, 302, 309, 313, 317, 343, 343a, 343b, 343c, and 343d as depicted in Table C1, or pharmaceutically acceptable salts thereof, or
the compound is selected from the group consisting of 101, 102, 103, 104, 105, 106, 107, 116, 116a, 117, 117a, 120, 121, 122, 124, 124a, 125, 125a, 126, 127, 128, 129, 130, 131, 131a, 132, 134, 156, 158, 159, 159a, 160, 160a, 160b, 162, 162a, 162b, 164, 164a, 164b, 166, 167, 167a, 167b, 169, 169a, 169b, 170, 173, 173a, 173b, 177, 177a, 180, 180a, 180b, 181, 186, 186a, 187, 187a, 187b, 193, 193a, 193b, 196, 197, 197a, 197b, 202, 202a, 202b, 203, 207, 207a, 207b, 209, 209a, 210, 210a, 213, 216, 216a, 216b, 218, 218a, 219, 219a, 219b, 226, 229, 231, 231a, 231b, 240, 240a, 240b, 243, 245, 245a, 245b, 246, 246a, 246b, 262, 262a, 262b, 263, 263a, 263b, 267, 268, 268a, 268b, 272, 272a, 272b, 277, 280, 286, 289, 289a, 289b, 292, 295, 302, 309, 313, and 317, as depicted in Table C1, or pharmaceutically acceptable salts thereof; or
the compound is selected from the group consisting of 101, 102, 103, 104, 105, 106, 107, 116, 116a, 117, 117a, 120, 121, 122, 124, 124a, 125, 125a, 126, 127, 128, 129, 130, 131, 131a, 132, 134, 156, 158, 159, 159a, 160, 160a, 160b, 162, 162a, 162b, 164, 164a, 164b, 166, 167, 167a, 167b, 169, 169a, 169b, 170, 173, 173a, 173b, 177, 177a, 180, 180a, 180b, 181, 186, 186a, 187, 187a, 187b, 193, 193a, 193b, 196, 197, 197a, 197b, 202, 202a, 202b, 203, 207, 207a, 207b, 209, 209a, 210, 210a, 213, 216, 216a, 216b, 218, 218a, 219, 219a, 219b, 226, 229, 231, 231a, 231b, 240, 240a, 240b, 243, 245, 245a, 245b, 246, 246a, 246b, 262, 262a, 262b, 263, 263a, 263b, 267, 268, 268a, 268b, 272, 272a, 272b, 277, 280, 286, 289, 289a, and 289b as depicted in Table C1, or a pharmaceutically acceptable salt thereof; or
the compound is selected from the group consisting of 101, 102, 103, 104, 105, 106, 107, 116, 116a, 117, 117a, 120, 121, 122, 124, 124a, 125, 125a, 126, 127, 128, 129, 130, 131, 131a, 132, 134, 156, 158, 159, 159a, 160, 160a, 160b, 162, 162a, 162b, 164, 164a, 164b, 166, 167, 167a, 167b, 169, 169a, 169b, 170, 173, 173a, 173b, 177, 177a, 180, 180a, 180b, 181, 186, 186a, 187, 187a, 187b, 193, 193a, 193b, 196, 197, 197a, 197b, 202, 202a, 202b, 203, 207, 207a, 207b, 209, 209a, 210, 210a, 213, 216, 216a, 216b, 218, 218a, 219, 219a, 219b, 226, 229, 231, 231a, 231b, 240, 240a, and 240b as depicted in Table C1, or a pharmaceutically acceptable salt thereof; or
the compound is selected from the group consisting of 101, 102, 103, 104, 105, 106, 107, 116, 116a, 117, 117a, 120, 121, 122, 124, 124a, 125, 125a, 126, 127, 128, 129, 130, 131, 131a, 132, 134, and 156 as depicted in Table C1, or a pharmaceutically acceptable salt thereof.

Embodiment 43. A pharmaceutical composition comprising the compound of any one of Embodiments 1-42, or a pharmaceutically acceptable salt thereof, and a pharmaceutically acceptable excipient.

Embodiment 44. A compound of any one of Embodiments 1-42, or a pharmaceutically acceptable salt thereof, or the pharmaceutical composition of Embodiment 43 for use in treatment.

Embodiment 45. A compound of any one of Embodiments 1-42 or a pharmaceutically acceptable salt thereof, or the pharmaceutical composition of Embodiment 43 for use in the treatment of a cancer selected from the group consisting of essential thrombocythemia, polycythemia vera, myelofibrosis, and a combination thereof.

Embodiment 46. The compound or pharmaceutical composition of Embodiment 45 and an additional therapeutic agent or therapy selected from the group consisting of fedratinib (e.g., fedratinib dihydrochloride monohydrate), momelotinib (e.g., momelotinib dihydrochloride), pacritinib (e.g., pacritinib citrate), ruxolitinib (e.g., ruxolitinib phosphate), and a combination thereof for use in treatment.

Embodiment 47. The compound or pharmaceutical composition of Embodiment 46 and an additional therapeutic agent or therapy selected from the group consisting of fedratinib (e.g., fedratinib dihydrochloride monohydrate), momelotinib (e.g., momelotinib dihydrochloride), pacritinib (e.g., pacritinib citrate), ruxolitinib (e.g., ruxolitinib phosphate), and a combination thereof for use in treatment of a cancer selected from the group consisting of essential thrombocythemia, polycythemia vera, myelofibrosis, and a combination thereof.

Embodiment 48. A compound of any one of Embodiments 1-42, or a pharmaceutically acceptable salt thereof, or the pharmaceutical composition of Embodiment 43 for use in a method of treating cancer selected from the group consisting of essential thrombocythemia, polycythemia vera, myelofibrosis, and a combination thereof, the method comprising administering to a subject a therapeutically effective amount of a compound of Embodiments 1-42, or a pharmaceutically acceptable salt thereof, or the pharmaceutical composition of Embodiment 43.

Embodiment 49. The compound or pharmaceutical composition of Embodiment 48, wherein the method comprises administering to the subject a therapeutically effective amount of an additional therapeutic agent or therapy selected from the group consisting of fedratinib (e.g., fedratinib dihydrochloride monohydrate), momelotinib (e.g., momelotinib dihydrochloride), pacritinib (e.g., pacritinib citrate), ruxolitinib (e.g., ruxolitinib phosphate), and a combination thereof.

Embodiment 50. The compound or pharmaceutical composition of Embodiment 49, wherein the compound of any one of Embodiments 1-42, or a pharmaceutically acceptable salt thereof, or the pharmaceutical composition of Embodiment 43, and the additional therapeutic agent or therapy are administered simultaneously, separately, or sequentially.

### Exemplary Formula (I-E) Embodiments

Embodiment 1. A compound of Formula (I-E):

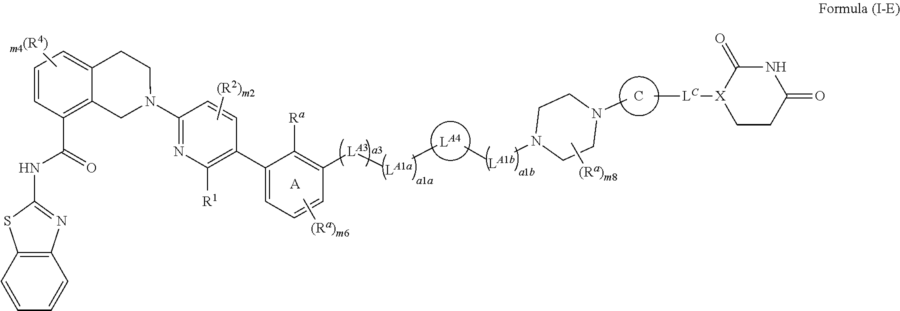

or a pharmaceutically acceptable salt thereof, wherein:
R¹ is selected from the group consisting of:
(a) C(O)OH,
(b) C(O)NR^{d}R^{e}, and
(c) C(O)OC₁₋₆ alkyl, wherein the C₁₋₆ alkyl is optionally substituted with 1-3 R^{e},
each R² and R⁴ is independently selected from the group consisting of: halo, CN, C₁₋₃ alkyl, C₁₋₃ haloalkyl, C₁₋₃ alkoxy, C₁₋₃ haloalkoxy, OH, and NR^{d}R^{e};
m2 is 0, 1, or 2;
m4 is 0, 1, 2, or 3;
m6 is 0 or 1;
a3 is F or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
wherein each R^{L} is independently selected from the group consisting of: halo; cyano; —OH; —C₁₋₆ alkoxy; —C₁₋₆ haloalkoxy; —NR^{d}R^{e}; C(═O)N(R^{f})₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{c};
m8 is 0, 1, or 2;
Ring C is selected from the group consisting of:

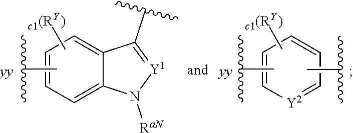

c1 is 0, 1, 2, or 3;
each R^{Y} is independently selected from the group consisting of R^{a} and R^{b};
R^{aN} is H or C₁₋₆ alkyl optionally substituted with 1-3 R^{e};
Y¹ and Y² are independently N, CH, or CR^{Y};
yy represents the point of attachment to L;
X is CH, C, or N;
the [symbol] is a single bond or a double bond;
L^{C} is selected from the group consisting of: a bond, —CH₂—, —CHR^{a}—, —C(R^{a})₂—, —N(R^{d})—, and O, provided that when X is N, then L^{C} is other than O;
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—; —N(H)—; —N(C₁₋₃ alkyl)-; —S(O)₀₋₂—; C(═O); and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(Rr)₂; S(O)₁₋₂(C₁₋₆ alkyl); S(O)₁₋₂(C₁₋₆ haloalkyl); S(O)₁₋₂N(R^{f})₂; and C₁₋₆ alkyl optionally substituted with 1-3 R^{h}.
each R^{f} is independently selected from the group consisting of: H and C₁₋₆ alkyl optionally substituted with 1-3 R^{h};
each R^{g} is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂.

Embodiment 2. The compound of Embodiment 1, wherein m8 is 0.

Embodiment 3. The compound of Embodiment 1 or 2, wherein a3 is 1.

Embodiment 4. The compound of any one of Embodiments 1-3, wherein L^{A3} is —O—.

Embodiment 5. The compound of any one of Embodiments 1-4, wherein a1a+a1b is 3.

Embodiment 6. The compound of any one of Embodiments 1-4, wherein a1a+a1b is 4.

Embodiment 7. The compound of any one of any one of Embodiments 1-4, wherein a1a+a1b is 2 or 5.

Embodiment 8. The compound of any one of Embodiments 1-7, wherein each occurrence of L^{A1a} and L^{A1b} is —CH₂—.

Embodiment 9. The compound of any one of Embodiments 1-7, wherein one occurrence of L^{A1a} is —CHR^{L}— or —C(R^{L})₂—; each remaining occurrence of L^{A1a} is —CH₂—; and each occurrence of L^{A1b} is —CH₂—.

Embodiment 10. The compound of any one of Embodiments 1-7, wherein one occurrence of L^{A1}b is —CHR^{L}— or —C(R^{L})₂—; each remaining occurrence of L^{A1b} is —CH₂—; and each occurrence of L^{A1a} is —CH₂—.

Embodiment 11. The compound of any one of Embodiments 1-10, wherein L^{A4} is 4-10 membered heterocyclylene optionally substituted with 1-3 R^{a}.

Embodiment 12. The compound of any one of Embodiments 1-11, wherein L^{A4} is selected from the group consisting of:

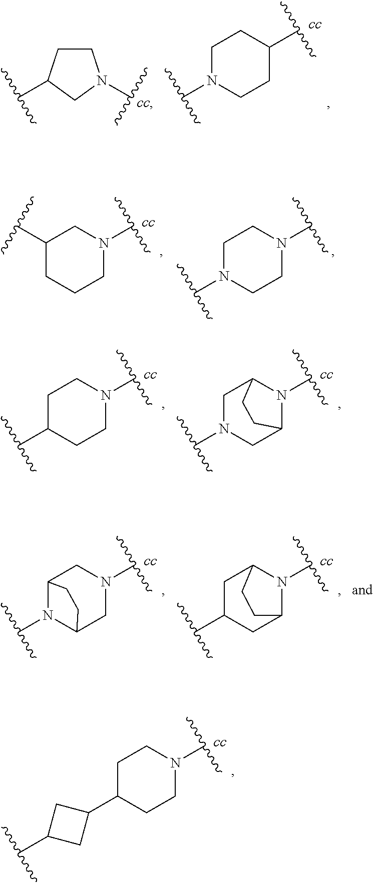

each optionally substituted with 1-3 R^{a} at one or more ring carbon atoms, wherein cc represents the point of attachment to L^{A1b}; or
wherein L^{A4} is selected from the group consisting of:

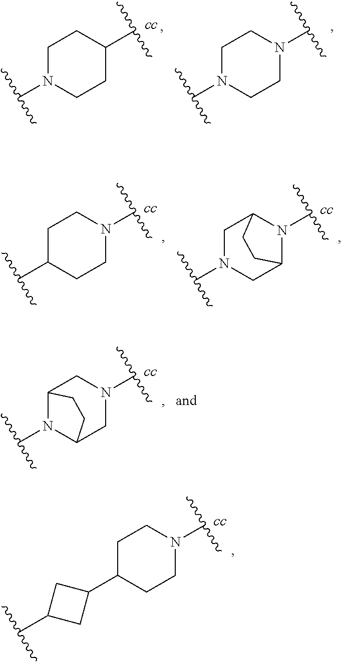 [SMILES: *C1CCN(*)CC1 ; *N1CCN(*)CC1 ; *C1CCN(*)CC1 ; *N1CC2CCC(C1)N2* ; *N1CC2CCC(C1)N2* ; *C1CC(C2CCN(*)CC2)C1]

each optionally substituted with 1-3 R^{a} at one or more ring carbon atoms, wherein cc represents the point of attachment to L^{A1b}.

Embodiment 13. The compound of Embodiments 11 or 12, wherein each R^{a} present on L^{A4} is independently a C₁₋₃ alkyl optionally substituted with 1-3 F.

Embodiment 14. The compound of any one of Embodiments 1-10, wherein L^{A4} is C₃₋₁₀ cycloalkylene optionally substituted with 1-3 R^{a}.

Embodiment 15. The compound of any one of Embodiments 1-10 or 14, wherein L^{A4} is 1,4-cyclohexylene optionally substituted with 1-3 R^{a}.

Embodiment 16. The compound of any one of Embodiments 1-10, wherein L^{A4} is phenylene or 5-6 membered heteroarylene, each of which is optionally substituted with 1-3 R^{a}.

Embodiment 17. The compound of any one of Embodiments 1-10 or 16, wherein L^{A4} is 1,4-phenylene optionally substituted with 1-3 R^{a}.

Embodiment 18. The compound of any one of Embodiments 1-10 or 16, wherein L^{A4} is 1,2-phenylene or 1,3-phenylene, each of which is optionally substituted with 1-3 R^{a}.

Embodiment 19. The compound of any one of Embodiments 1-18, wherein m6 is 0.

Embodiment 20. The compound of any one of Embodiments 1-19, wherein the R^{a} present on Ring A is C₁₋₃ alkyl optionally substituted with 1-3-F.

Embodiment 21. The compound of any one of Embodiments 1-20, wherein R¹ is C(O)OH.

Embodiment 22. The compound of any one of Embodiments 1-21, wherein m2 is 0.

Embodiment 23. The compound of any one of Embodiments 1-22, wherein m4 is 0.

Embodiment 24. The compound of any one of Embodiments 1-23, wherein Ring C is

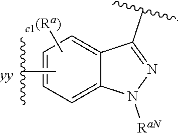

Embodiment 25. The compound of any one of Embodiments 1-24, wherein Ring C is

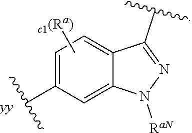

Embodiment 26. The compound of any one of Embodiments 1-24, wherein Ring C is

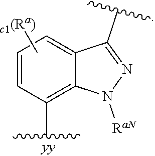

Embodiment 27. The compound of any one of Embodiments 1-26, wherein R^{aN} is C₁₋₃ alkyl.

Embodiment 28. The compound of any one of Embodiments 1-27, wherein R^{aN} is methyl.

Embodiment 29. The compound of any one of Embodiments 1-27, wherein c1 is 0.

Embodiment 30. The compound of any one of Embodiments 1-29, wherein L^{C} is a bond.

Embodiment 31. The compound of any one of Embodiments 1-30, wherein X is CH.

Embodiment 32. The compound of any one of Embodiments 1-23, wherein the

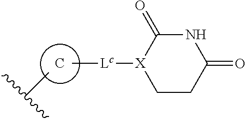

moiety is

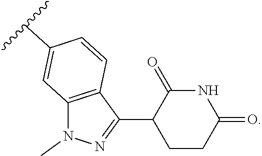 [SMILES: *c1ccc2c(C3CCC(=O)NC3=O)nn(C)c2c1]

Embodiment 33. The compound of any one of Embodiments 1-23, wherein the

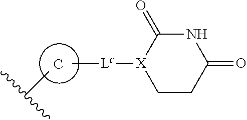

moiety is

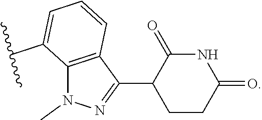 [SMILES: *c1cccc2c(C3CCC(=O)NC3=O)nn(C)c12]

Embodiment 34. The compound of Embodiment 1, wherein the compound is a compound of Formula (I-E-1):

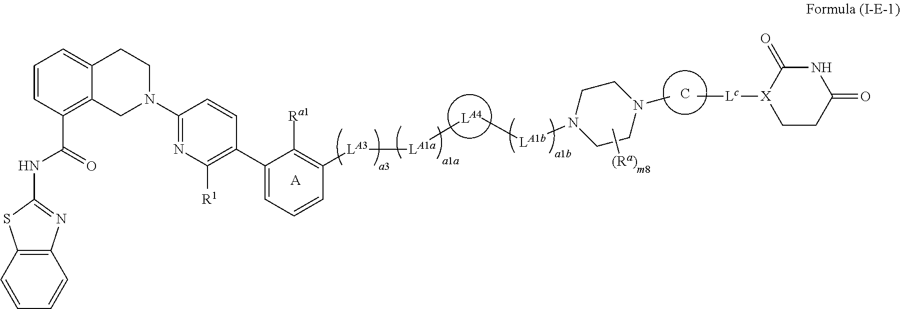

or pharmaceutically acceptable salts thereof, wherein:
R¹ is C(O)OH or C(O)OC₁₋₆ alkyl;
R^{a1} is C₁₋₃ alkyl optionally substituted with 1-3 F;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
m8 is 0, 1, or 2.

Embodiment 35. The compound of Embodiment 1, wherein the compound is a compound of Formula (I-E-2):

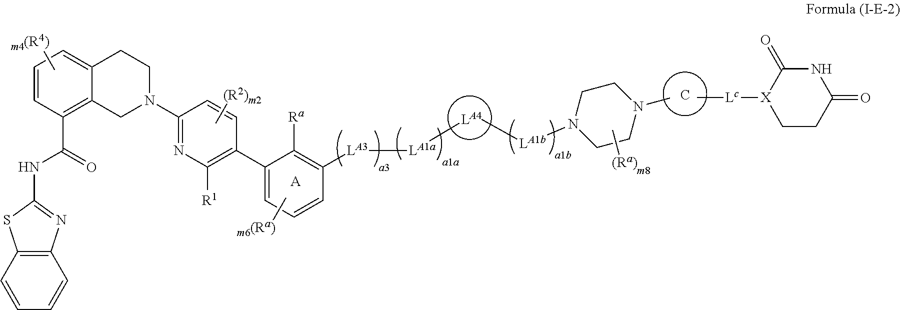

or pharmaceutically acceptable salts thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
m8 is 0, 1, or 2; and
Ring C is

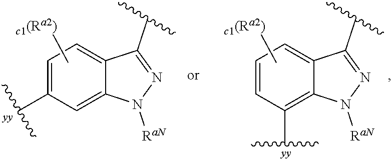

wherein: c1 is 0 or 1; R^{a2} is selected from the group consisting of: halo (e.g., —F) and C₁₋₃ alkyl optionally substituted with 1-3 F; and R^{aN} is C₁₋₃ alkyl.

Embodiment 36. The compound of Embodiments 1 or 34-35, wherein the compound is a compound of Formula (I-E-3):

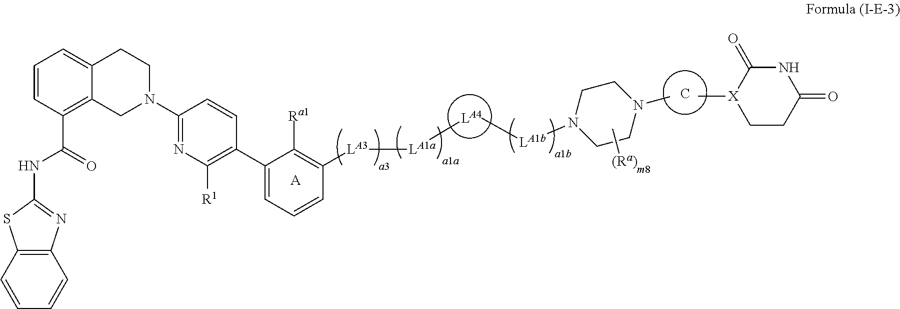

or pharmaceutically acceptable salts thereof, wherein:
R¹ is C(O)OH or C(O)OC₁₋₆ alkyl;
R^{a1} is C₁₋₃ alkyl optionally substituted with 1-3 F;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
m8 is 0, 1, or 2; and
Ring C is

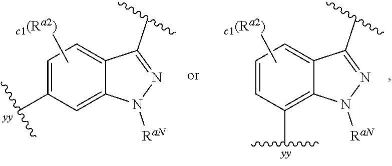

wherein: c1 is 0 or 1; R^{a2} is selected from the group consisting of: halo (e.g., —F) and C₁₋₃ alkyl optionally substituted with 1-3 F; and R^{aN} is C₁₋₃ alkyl.

Embodiment 37. The compound of any one of Embodiments 34-36, wherein m8 is 0.

Embodiment 38. The compound of any one of Embodiments 1 or 34-37, wherein the

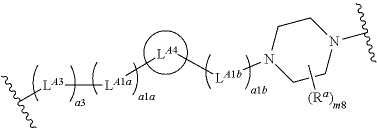

moiety is selected from the groups depicted in Table (L-I-E):

TABLE (L-I-E)

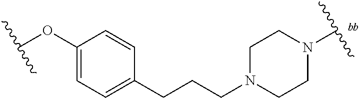

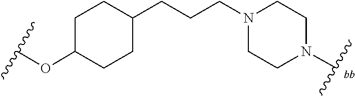

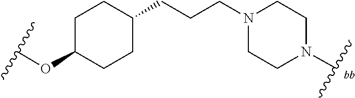

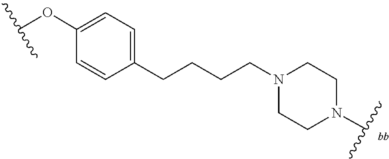

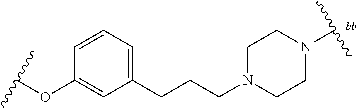

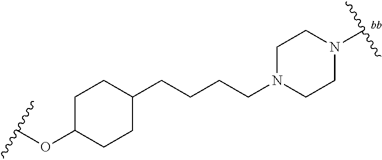

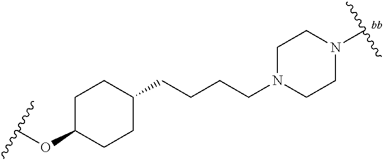

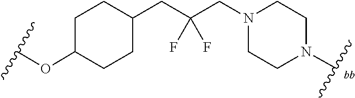

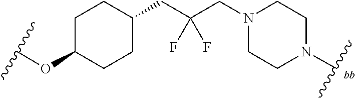

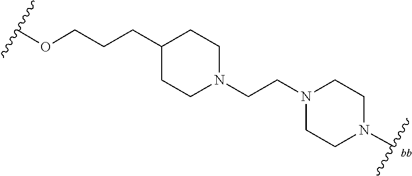

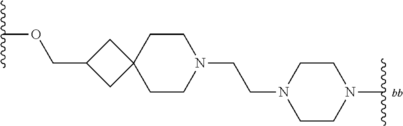

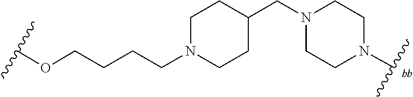

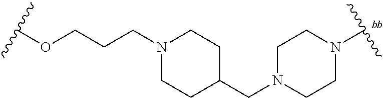

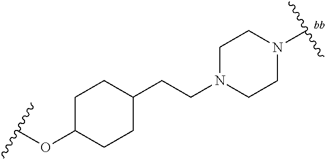

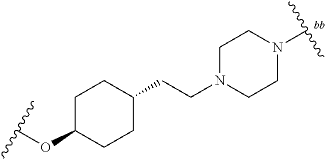

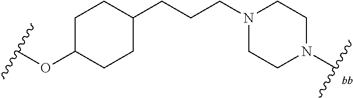

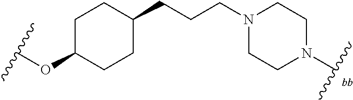

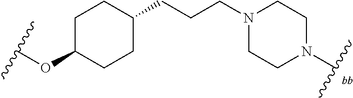

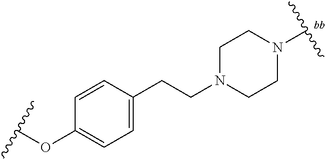

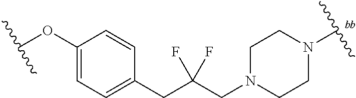

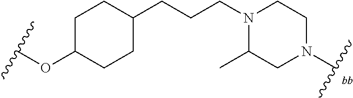

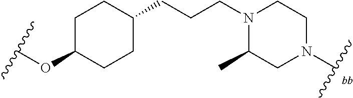

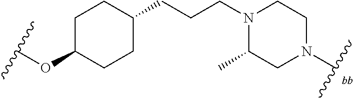

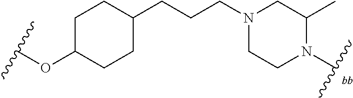

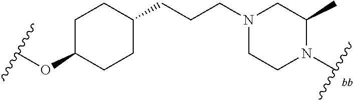

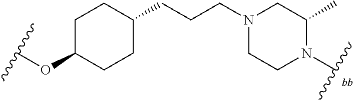

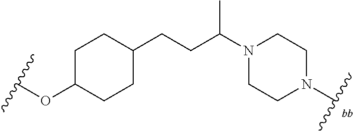

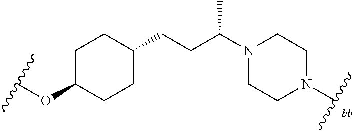

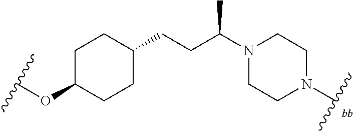

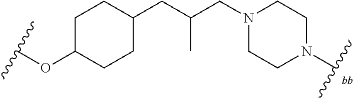

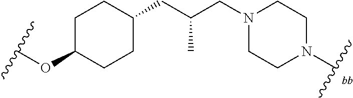

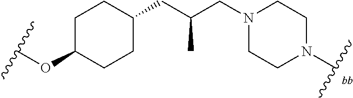

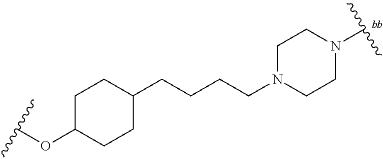

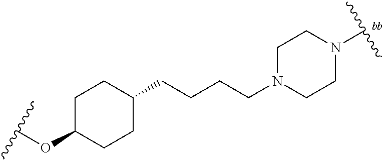

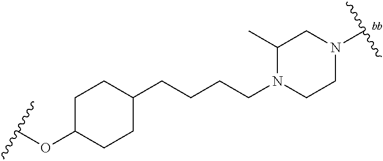

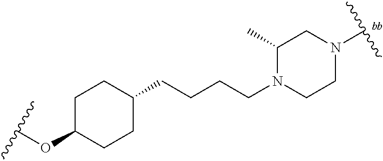

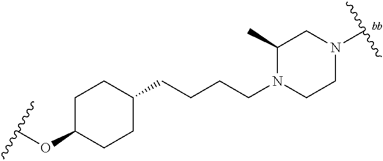

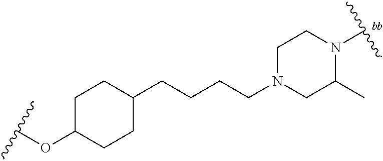

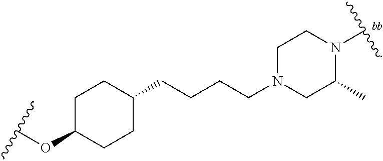

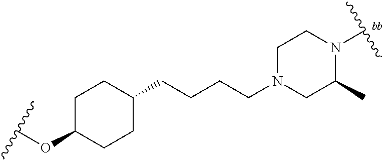

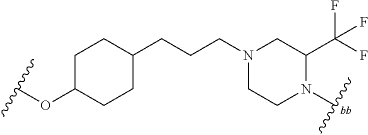

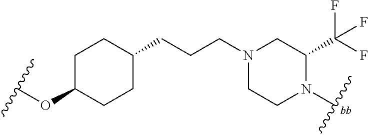

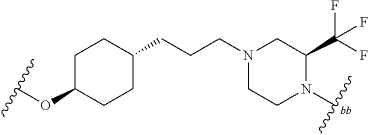

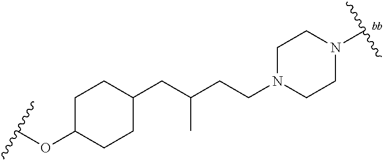

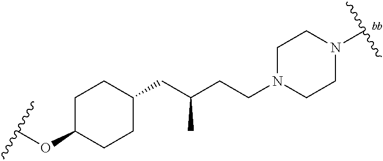

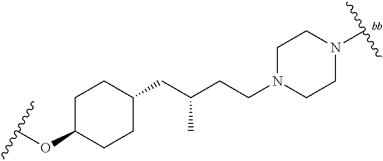

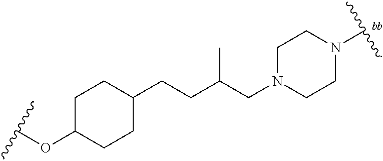

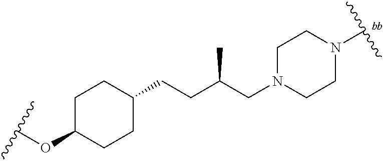

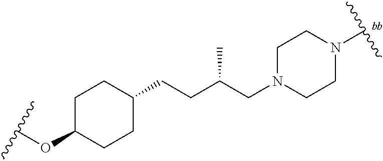

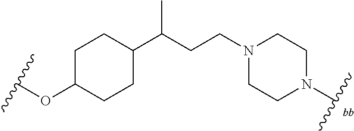

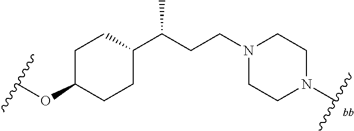

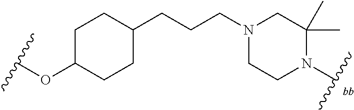

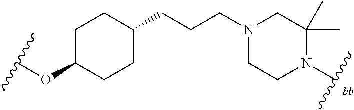

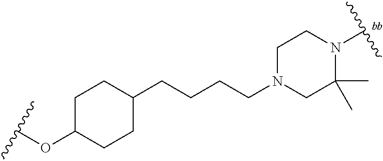

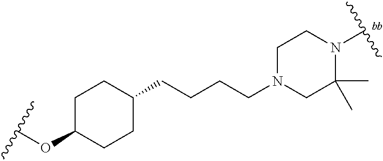

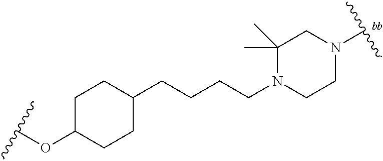

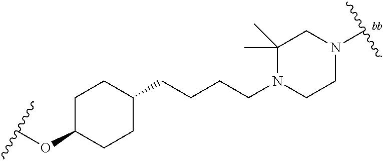

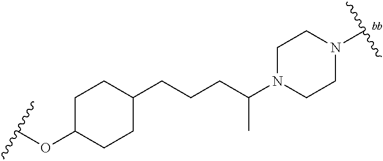

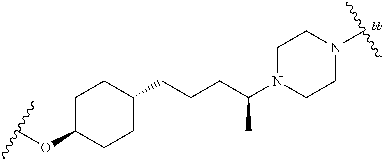

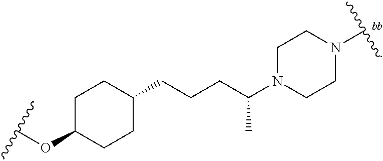

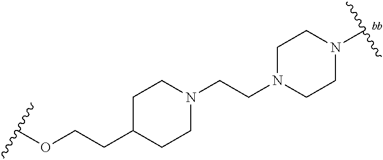

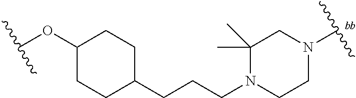

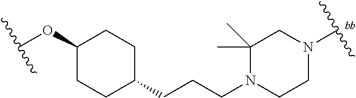

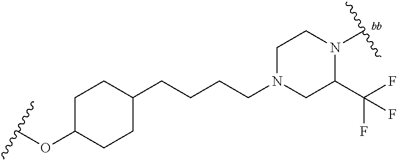

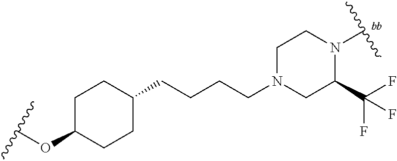

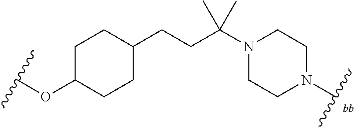

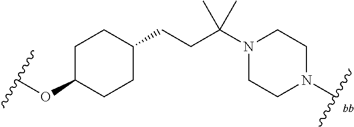

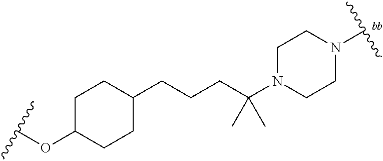

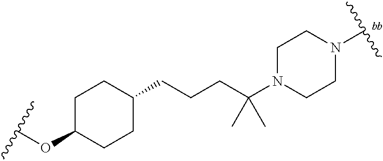

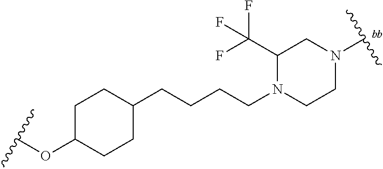

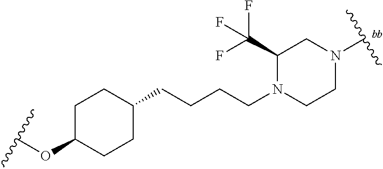

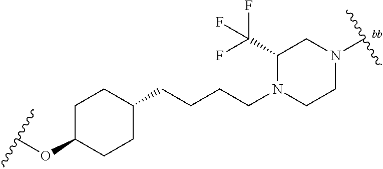

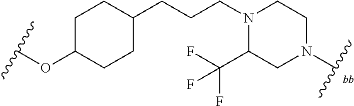

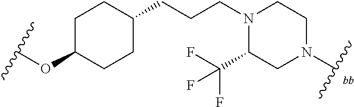

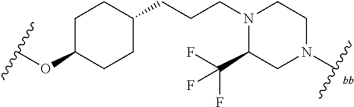

wherein bb represents the point of attachment to Ring C; or
wherein the

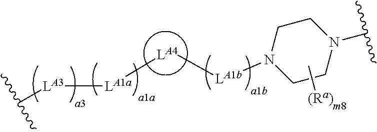

moiety is selected from the group consisting of:

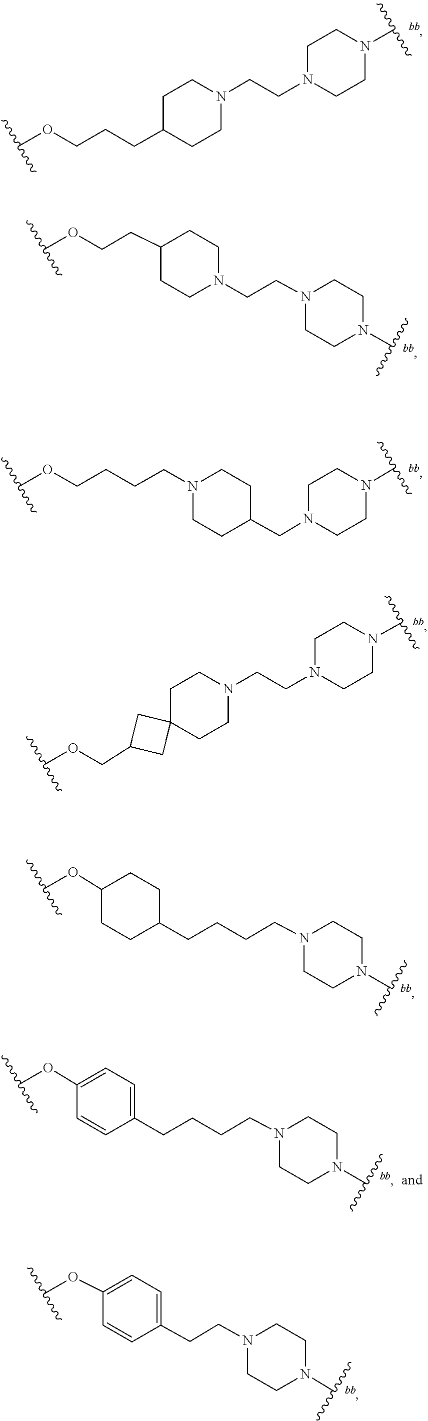

wherein bb represents the point of attachment to Ring C.

Embodiment 39. The compound of any one of Embodiments 1-36, wherein the compound is selected from the group consisting of 147, 147a, 147b, 148, 148a, 148b, 149, 150, 161, 161a, 163, 168, 168a, 175, 188, 195, 200, 201, 204, 204a, 208, 211, 211a, 212, 212a, 215, 215a, 220, 225, 230, 232, 233, 234, 235, 241, 273, 273a, 273b, 274, 274a, 274b, 275, 275a, 275b, 278, 278a, 278b, 281, 281a, 281b, 282, 282a, 282b, 283, 283a, 283b, 284, 284a, 284b, 293, 293a, 296, 296a, 297, 297a, 297b, 300, 300a, 300b, 301, 301a, 301b, 307, 307a, 307b, 308, 308a, 308b, 310, 310a, 316, 316a, 318, 318a, 319, 319a, 322, 322a, 323, 323a, 324, 324a, 325, 325a, 328, 328a, 333, 333a, 334, 334a, 335, 335a, 336, 336a, 339, 339a, 339b, 340, 340a, 341, 341a, 342, 342a, 345, 345a, 346, 346a, 346b, 347, 347a, 347b, 349, 349a, 349b, 350, 350a, 354, 354a, and 354b as depicted in Table C1, or pharmaceutically acceptable salts thereof; or
the compound is selected from the group consisting of 147, 147a, 147b, 148, 148a, 148b, 149, 150, 161, 161a, 163, 168, 168a, 175, 188, 195, 200, 201, 204, 204a, 208, 211, 211a, 212, 212a, 215, 215a, 220, 225, 230, 232, 233, 234, 235, 241, 273, 273a, 273b, 274, 274a, 274b, 275, 275a, 278, 278a, 278b, 281, 281a, 281b, 282, 282a, 282b, 283, 283a, 283b, 284, 284a, 284b, 293, 293a, 296, 296a, 297, 297a, 297b, 300, 300a, 300b, 301, 301a, 301b, 307, 307a, 307b, 308, 308a, 308b, 310, 310a, 316, 316a, 318, 318a, 319, 319a, 322, 322a, 323, 323a, 324, 324a, 325, 325a, 328, 328a, 333, 333a, 334, 334a, 335, 335a, 336, 336a, 339, 339a, 340, 340a, 341, 341a, 342, 342a, 345, 345a, 346, 346a, 346b, 347, 347a, 347b, 349, 349a, 349b, 350, and 350a as depicted in Table C1, or pharmaceutically acceptable salts thereof; or
the compound is selected from the group consisting of 147, 147a, 147b, 148, 148a, 148b, 149, 150, 161, 161a, 163, 168, 168a, 175, 188, 195, 200, 201, 204, 204a, 208, 211, 211a, 212, 212a, 215, 215a, 220, 225, 230, 232, 233, 234, 235, 241, 273, 273a, 273b, 274, 274a, 274b, 275, 275a, 278, 278a, 278b, 281, 281a, 281b, 282, 282a, 282b, 283, 283a, 283b, 284, 284a, 284b, 293, 293a, 296, 296a, 297, 297a, 297b, 300, 300a, 300b, 301, 301a, 301b, 307, 307a, 307b, 308, 308a, 308b, 310, 310a, 316, 316a, 318, 318a, 319, 319a, 322, 322a, 323, 323a, 324, 324a, 325, and 325a, as depicted in Table C1, or pharmaceutically acceptable salts thereof; or
the compound is selected from the group consisting of 147, 147a, 147b, 148, 148a, 148b, 149, 150, 161, 161a, 163, 168, 168a, 175, 188, 195, 200, 201, 204, 204a, 208, 211, 211a, 212, 212a, 215, 215a, 220, 225, 230, 232, 233, 234, 235, 241, 273, 273a, 273b, 274, 274a, 274b, 275, 275a, 278, 278a, 278b, 281, 281a, 281b, 282, 282a, 282b, 283, 283a, 283b, 284, 284a, and 284b as depicted in Table C1, or pharmaceutically acceptable salts thereof; or
the compound is selected from the group consisting of 147, 147a, 148, 148a, 149, 150, 161, 161a, 163, 168, 168a, 175, 188, 195, 200, 201, 204, 204a, 208, 211, 211a, 212, 212a, 215, 215a, 220, 225, 230, 232, 233, 234, 235, and 241, as depicted in Table C1, or pharmaceutically acceptable salts thereof; or
the compound is selected from the group consisting of 147, 147a, 148, 148a, 149, and 150 as depicted in Table C1, or pharmaceutically acceptable salts thereof; or

Embodiment 40. A pharmaceutical composition comprising the compound of any one of Embodiments 1-39, or a pharmaceutically acceptable salt thereof, and a pharmaceutically acceptable excipient.

Embodiment 41. A compound of any one of Embodiments 1-39, or a pharmaceutically acceptable salt thereof, or the pharmaceutical composition of Embodiment 40 for use in treatment.

Embodiment 42. A compound of any one of Embodiments 1-39 or a pharmaceutically acceptable salt thereof, or the pharmaceutical composition of Embodiment 40 for use in the treatment of a cancer selected from the group consisting of essential thrombocythemia, polycythemia vera, myelofibrosis, and a combination thereof.

Embodiment 43. The compound or pharmaceutical composition of Embodiment 42 and an additional therapeutic agent or therapy selected from the group consisting of fedratinib (e.g., fedratinib dihydrochloride monohydrate), momelotinib (e.g., momelotinib dihydrochloride), pacritinib (e.g., pacritinib citrate), ruxolitinib (e.g., ruxolitinib phosphate), and a combination thereof for use in treatment.

Embodiment 44. The compound or pharmaceutical composition of Embodiment 43 and an additional therapeutic agent or therapy selected from the group consisting of fedratinib (e.g., fedratinib dihydrochloride monohydrate), momelotinib (e.g., momelotinib dihydrochloride), pacritinib (e.g., pacritinib citrate), ruxolitinib (e.g., ruxolitinib phosphate), and a combination thereof for use in treatment of a cancer selected from the group consisting of essential thrombocythemia, polycythemia vera, myelofibrosis, and a combination thereof.

Embodiment 45. A compound of any one of Embodiments 1-39, or a pharmaceutically acceptable salt thereof, or the pharmaceutical composition of Embodiment 40 for use in a method of treating cancer selected from the group consisting of essential thrombocythemia, polycythemia vera, myelofibrosis, and a combination thereof, the method comprising administering to a subject a therapeutically effective amount of a compound of Embodiments 1-39, or a pharmaceutically acceptable salt thereof, or the pharmaceutical composition of Embodiment 40.

Embodiment 46. The compound or pharmaceutical composition of Embodiment 45, wherein the method comprises administering to the subject a therapeutically effective amount of an additional therapeutic agent or therapy selected from the group consisting of fedratinib (e.g., fedratinib dihydrochloride monohydrate), momelotinib (e.g., momelotinib dihydrochloride), pacritinib (e.g., pacritinib citrate), ruxolitinib (e.g., ruxolitinib phosphate), and a combination thereof.

Embodiment 47. The compound or pharmaceutical composition of Embodiment 46, wherein the compound of any one of Embodiments 1-39, or a pharmaceutically acceptable salt thereof, or the pharmaceutical composition of Embodiment 40, and the additional therapeutic agent or therapy are administered simultaneously, separately, or sequentially.

### Exemplary Formula (I-Eb) Embodiments

Embodiment 1. A compound of Formula (I-Eb):

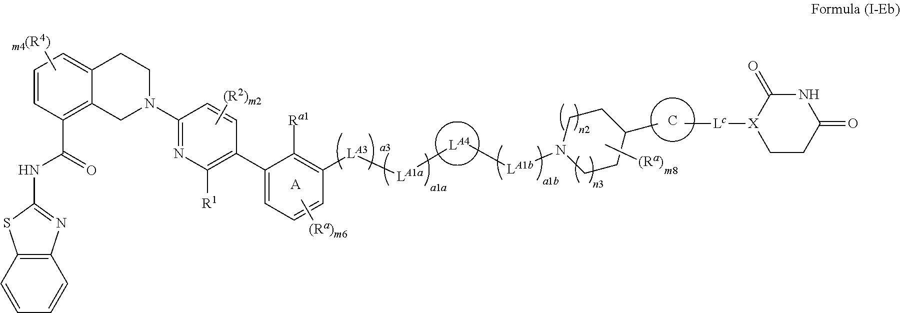

or pharmaceutically acceptable salts thereof, wherein.
R¹ is selected from the group consisting of:
(a) C(O)OH,
(b) C(O)NR^{d}R^{e}, and
(c) C(O)OC₁₋₆ alkyl, wherein the C₁₋₆ alkyl is optionally substituted with 1-3 R^{c},
each R² and R⁴ is independently selected from the group consisting of: halo, CN, C₁₋₃ alkyl, C₁₋₃ haloalkyl, C₁₋₃ alkoxy, C₁₋₃ haloalkoxy, OH, and NR^{d}R^{e};
m2 is 0, 1, or 2;
m4 is 0, 1, 2, or 3;
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
wherein each R^{L} is independently selected from the group consisting of: halo; cyano; —OH; —C₁₋₆ alkoxy; —C₁₋₆ haloalkoxy; —NR^{d}R^{e}; C(═O)N(R^{f})₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{c};
n2 and n3 are independently 0, 1, or 2; and
m8 is 0, 1, or 2.
Ring C is selected from the group consisting of:

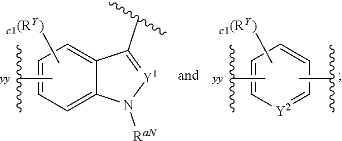

c1 is 0, 1, 2, or 3;
each R^{Y} is independently selected from the group consisting of: R^{a} and R^{b};
R^{aN} is H or C₁₋₆ alkyl optionally substituted with 1-3 R^{c};
Y¹ and Y² are independently N, CH, or CR^{Y};
yy represents the point of attachment to L;
X is CH, C, or N;
the [symbol] is a single bond or a double bond;
L^{C} is selected from the group consisting of: a bond, —CH₂—, —CHR^{a}—, —C(R^{a})₂—, —N(R^{d})—, and O, provided that when X is N, then L^{C} is other than 0;
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{e};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—; —N(H)—; —N(C₁₋₃ alkyl)-; —S(O)₀₋₂—; C(═O); and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(R^{f})₂; S(O)₁₋₂(C₁₋₆ alkyl); S(O)₁₋₂(C₁₋₆ haloalkyl); S(O)₁₋₂N(R^{f})₂; and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}; C₁-3 alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂.

Embodiment 2. The compound of Embodiment 1, wherein m8 is 0.

Embodiment 3. The compound of Embodiment 1 or 2, wherein a3 is 1.

Embodiment 4. The compound of any one of Embodiments 1-3, wherein L^{A3} is —O—.

Embodiment 5. The compound of any one of Embodiments 1-4, wherein a1a+a1b is 3.

Embodiment 6. The compound of any one of Embodiments 1-4, wherein a1a+a1b is 4.

Embodiment 7. The compound of any one of any one of Embodiments 1-4, wherein a1a+a1b is 2 or 5.

Embodiment 8. The compound of any one of Embodiments 1-7, wherein each occurrence of L^{A1a} and L^{A1b} is —CH₂—.

Embodiment 9. The compound of any one of Embodiments 1-7, wherein one occurrence of L^{A1a} is —CHR^{L}— or —C(R^{L})₂—; each remaining occurrence of L^{A1a} is —CH₂—; and each occurrence of L^{A1b} is —CH₂—.

Embodiment 10. The compound of any one of Embodiments 1-7, wherein one occurrence of L^{A1b} is —CHR^{L}— or —C(R^{L})₂—; each remaining occurrence of L^{A1b} is —CH₂—; and each occurrence of L^{A1a} is —CH₂—.

Embodiment 11. The compound of any one of Embodiments 1-10, wherein L^{A4} is 4-10 membered heterocyclylene optionally substituted with 1-3 R^{a}.

Embodiment 12. The compound of any one of Embodiments 1-11, wherein L^{A4} is selected from the group consisting of:

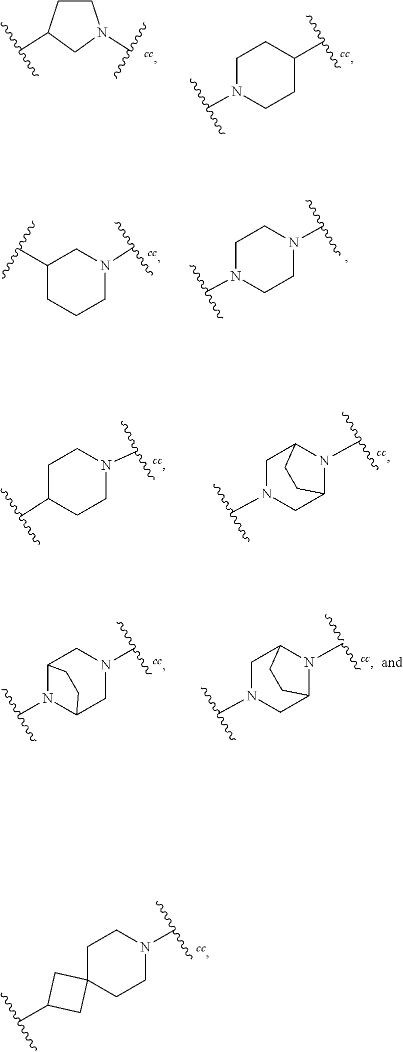

each optionally substituted with 1-3 R^{a} at one or more ring carbon atoms, wherein cc represents the point of attachment to L^{A1b}; or
wherein L^{A4} is selected from the group consisting of:

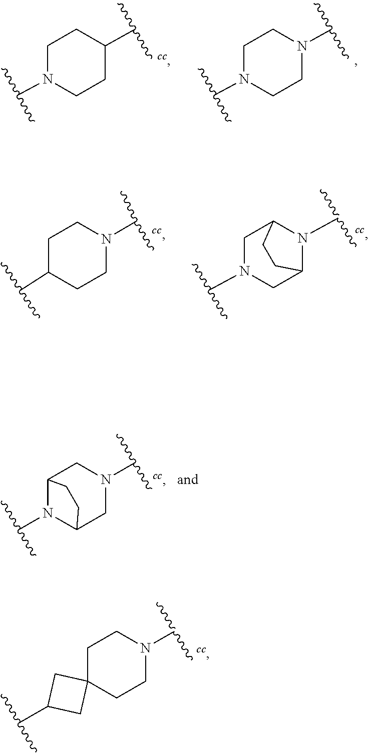 [SMILES: *C1CCN(*)CC1 ; *N1CCN(*)CC1 ; *C1CCN(*)CC1 ; *N1CC2CCC(C1)N2* ; *N1CC2CCC(C1)N2* ; *C1CC2(CCN(*)CC2)C1]

each optionally substituted with 1-3 R^{a} at one or more ring carbon atoms, wherein cc represents the point of attachment to L^{A1b}.

Embodiment 13. The compound of Embodiments 11 or 12, wherein each R^{a} present on L^{A4} is independently a C₁₋₃ alkyl optionally substituted with 1-3 F.

Embodiment 14. The compound of any one of Embodiments 1-10, wherein L^{A4} is C₃₋₁₀ cycloalkylene optionally substituted with 1-3 R^{a}.

Embodiment 15. The compound of any one of Embodiments 1-10 or 14, wherein L^{A4} is 1,4-cyclohexylene optionally substituted with 1-3 R^{a}.

Embodiment 16. The compound of any one of Embodiments 1-10, wherein L^{A4} is phenylene or 5-6 membered heteroarylene, each of which is optionally substituted with 1-3 R^{a}.

Embodiment 17. The compound of any one of Embodiments 1-10 or 16, wherein L^{A4} is 1,4-phenylene optionally substituted with 1-3 R^{a}.

Embodiment 18. The compound of any one of Embodiments 1-10 or 16, wherein L^{A4} is 1,2-phenylene or 1,3-phenylene, each of which is optionally substituted with 1-3 R^{a}.

Embodiment 19. The compound of any one of Embodiments 1-18, wherein m6 is 0.

Embodiment 20. The compound of any one of Embodiments 1-19, wherein the R^{a} present on Ring A is C₁₋₃ alkyl optionally substituted with 1-3-F.

Embodiment 21. The compound of any one of Embodiments 1-20, wherein R¹ is C(O)OH.

Embodiment 22. The compound of any one of Embodiments 1-21, wherein m2 is 0.

Embodiment 23. The compound of any one of Embodiments 1-22, wherein m4 is 0.

Embodiment 24. The compound of any one of Embodiments 1-23, wherein Ring C is

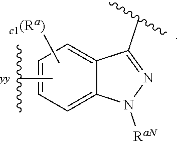

Embodiment 25. The compound of any one of Embodiments 1-24, wherein Ring C is

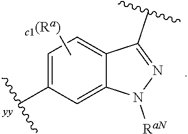

Embodiment 26. The compound of any one of Embodiments 1-24, wherein Ring C is

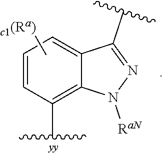

Embodiment 27. The compound of any one of Embodiments 1-26, wherein R^{aN} is C₁₋₃ alkyl.

Embodiment 28. The compound of any one of Embodiments 1-27, wherein R^{aN} is methyl.

Embodiment 29. The compound of any one of Embodiments 1-27, wherein c1 is 0.

Embodiment 30. The compound of any one of Embodiments 1-29, wherein L^{C} is a bond.

Embodiment 31. The compound of any one of Embodiments 1-30, wherein X is CH.

Embodiment 32. The compound of any one of Embodiments 1-23, wherein the

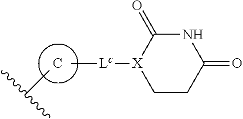

moiety is

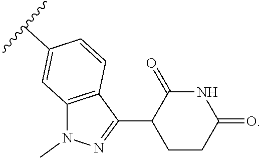 [SMILES: *c1ccc2c(C3CCC(=O)NC3=O)nn(C)c2c1]

Embodiment 33. The compound of any one of Embodiments 1-23, wherein the

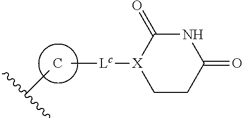

moiety is

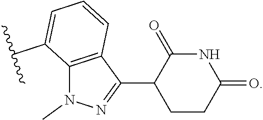 [SMILES: *c1cccc2c(C3CCC(=O)NC3=O)nn(C)c12]

Embodiment 34. The compound of Embodiment 1, wherein the compound is a compound of Formula (I-Eb-1):

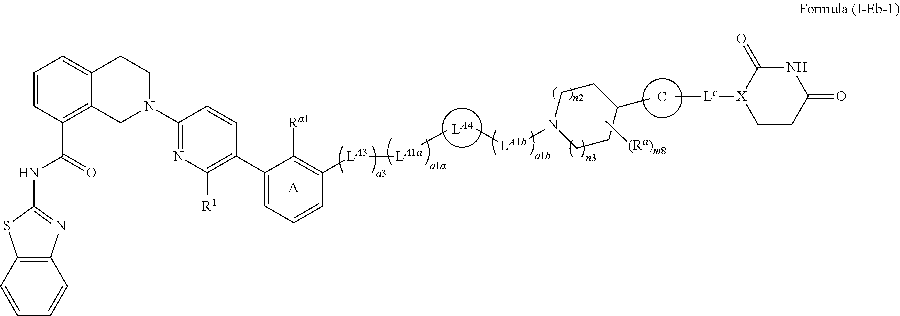

or pharmaceutically acceptable salts thereof, wherein:
R¹ is C(O)OH or C(O)OC₁₋₆ alkyl;
R^{a1} is C₁₋₃ alkyl optionally substituted with 1-3 F;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
n2 and n3 are independently 0, 1, or 2; and
m8 is 0, 1, or 2.

Embodiment 35. The compound of Embodiment 1, wherein the compound is a compound of Formula (I-Eb-2):

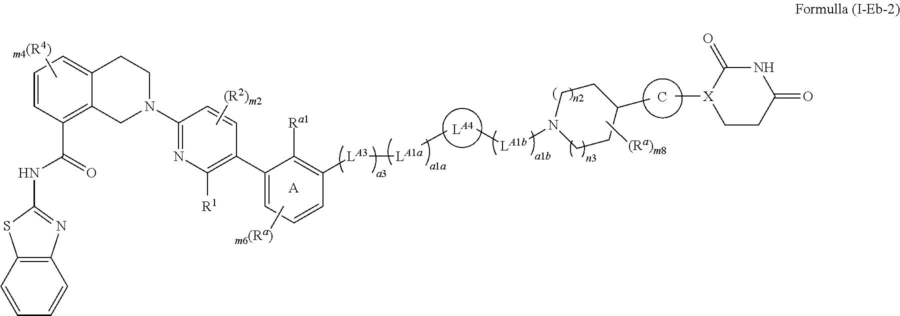

or pharmaceutically acceptable salts thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
m8 is 0, 1, or 2;
n2 and n3 are independently 0, 1, or 2; and
Ring C is

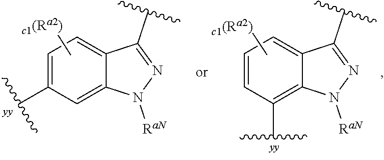

wherein: c1 is 0 or 1; R^{a2} is selected from the group consisting of: halo (e.g., —F) and C₁₋₃ alkyl optionally substituted with 1-3 F; and R^{aN} is C₁₋₃ alkyl.

Embodiment 36. The compound of any one of Embodiments 1, 31, or 32, wherein the compound is a compound of Formula (I-Eb-3):

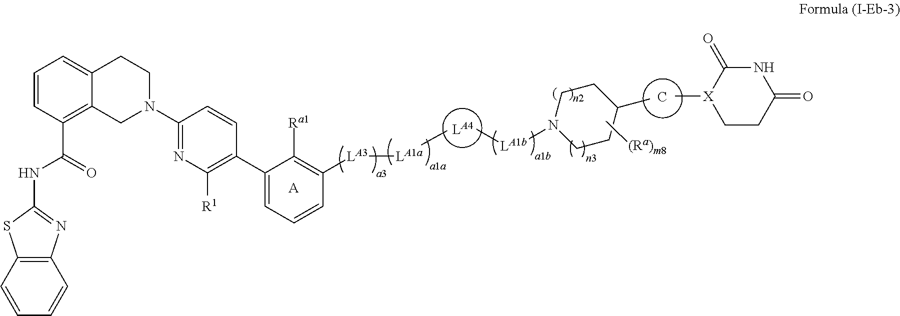

or pharmaceutically acceptable salts thereof, wherein:
R¹ is C(O)OH or C(O)OC₁₋₆ alkyl;
R^{a1} is C₁₋₃ alkyl optionally substituted with 1-3 F;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
m8 is 0, 1, or 2;
n2 and n3 are independently 0, 1, or 2; and
Ring C is

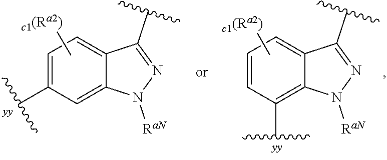

wherein: c1 is 0 or 1; R^{a2} is selected from the group consisting of: halo (e.g., —F) and C₁₋₃ alkyl optionally substituted with 1-3 F; and R^{aN} is C₁₋₃ alkyl.

In some embodiments of Formula (I-Eb-1), (I-Eb-2), or (I-Eb-3):
a3 is 1;
L^{A3} is —O—;
a1a is 0; and
L^{A4} is selected from the group consisting of:
C₃₋₁₀ cycloalkylene optionally substituted with 1-3 R^{a}; and
phenylene optionally substituted with 1-3 R^{a}.

Embodiment 37. The compound of any one of Embodiments 1-36, wherein the

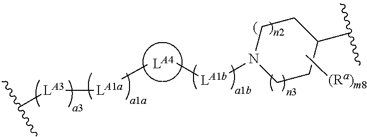

moiety is selected from the groups depicted in Table (L-I-Eb):

TABLE (L-I-Eb)

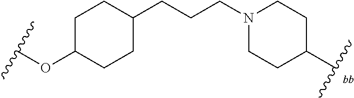

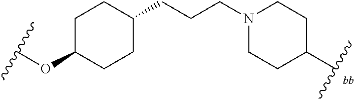

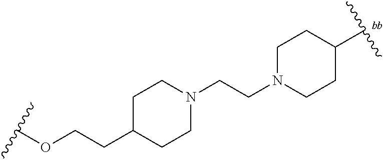

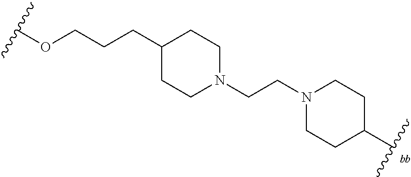

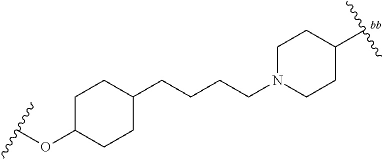

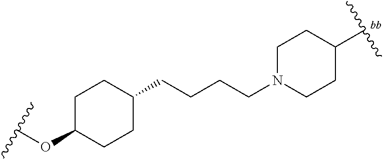

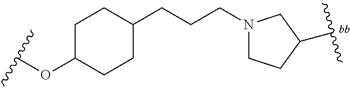

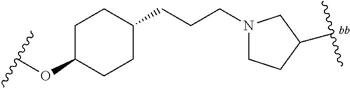

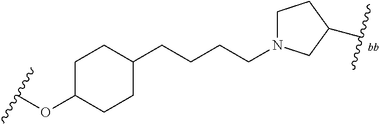

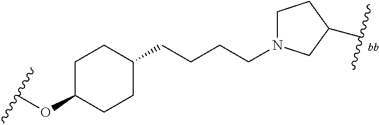

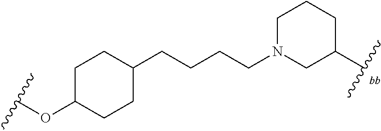

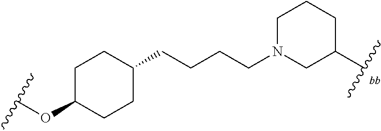

wherein bb represents the point of attachment to Ring C.

Embodiment 38. The compound of any one of Embodiments 1-34, wherein the compound is selected from the group consisting of 248, 249, 255, 256, 269, 269a, 270, 270a, 298, 298a, 299, 299a, 320, 320a, 320b, 321, 321a, 321b, 331, 331a, 331b, 332, 332a, 332b, 351, 351a, 351b, 352, 352a, and 352b as depicted in Table C1, or pharmaceutically acceptable salts thereof, or
the compound is selected from the group consisting of 248, 249, 255, 256, 269, 269a, 270, 270a, 298, 298a, 299, 299a, 320, 320a, 320b, 321, 321a, and 321b, as depicted in Table C1, or pharmaceutically acceptable salts thereof, or
the compound is selected from the group consisting of 248, 249, 255, 256, 269, 269a, 270, and 270a as depicted in Table C1, or pharmaceutically acceptable salts thereof.

Embodiment 39. A pharmaceutical composition comprising the compound of any one of Embodiments 1-38, or a pharmaceutically acceptable salt thereof, and a pharmaceutically acceptable excipient.

Embodiment 40. A compound of any one of Embodiments 1-38, or a pharmaceutically acceptable salt thereof, or the pharmaceutical composition of Embodiment 39 for use in treatment.

Embodiment 41. A compound of any one of Embodiments 1-38 or a pharmaceutically acceptable salt thereof, or the pharmaceutical composition of Embodiment 39 for use in the treatment of a cancer selected from the group consisting of essential thrombocythemia, polycythemia vera, myelofibrosis, and a combination thereof.

Embodiment 42. The compound or pharmaceutical composition of Embodiment 41 and an additional therapeutic agent or therapy selected from the group consisting of fedratinib (e.g., fedratinib dihydrochloride monohydrate), momelotinib (e.g., momelotinib dihydrochloride), pacritinib (e.g., pacritinib citrate), ruxolitinib (e.g., ruxolitinib phosphate), and a combination thereof for use in treatment.

Embodiment 43. The compound or pharmaceutical composition of Embodiment 42 and an additional therapeutic agent or therapy selected from the group consisting of fedratinib (e.g., fedratinib dihydrochloride monohydrate), momelotinib (e.g., momelotinib dihydrochloride), pacritinib (e.g., pacritinib citrate), ruxolitinib (e.g., ruxolitinib phosphate), and a combination thereof for use in treatment of a cancer selected from the group consisting of essential thrombocythemia, polycythemia vera, myelofibrosis, and a combination thereof.

Embodiment 44. A compound of any one of Embodiments 1-38, or a pharmaceutically acceptable salt thereof, or the pharmaceutical composition of Embodiment 39 for use in a method of treating cancer selected from the group consisting of essential thrombocythemia, polycythemia vera, myelofibrosis, and a combination thereof, the method comprising administering to a subject a therapeutically effective amount of a compound of Embodiments 1-38, or a pharmaceutically acceptable salt thereof, or the pharmaceutical composition of Embodiment 39.

Embodiment 45. The compound or pharmaceutical composition of Embodiment 44, wherein the method comprises administering to the subject a therapeutically effective amount of an additional therapeutic agent or therapy selected from the group consisting of fedratinib (e.g., fedratinib dihydrochloride monohydrate), momelotinib (e.g., momelotinib dihydrochloride), pacritinib (e.g., pacritinib citrate), ruxolitinib (e.g., ruxolitinib phosphate), and a combination thereof.

Embodiment 46. The compound or pharmaceutical composition of Embodiment 45, wherein the compound of any one of Embodiments 1-38, or a pharmaceutically acceptable salt thereof, or the pharmaceutical composition of Embodiment 39, and the additional therapeutic agent or therapy are administered simultaneously, separately, or sequentially.

### Exemplary Formula (I) Embodiments

Embodiment 1. A compound of Formula (I):

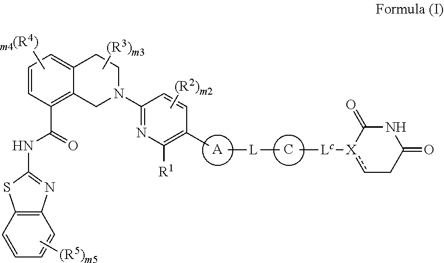

or a pharmaceutically acceptable salt thereof, wherein:
Ring A is selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) phenylene or 5-6 membered heteroarylene, each of which is optionally substituted with 1-3 substituents independently selected from the group consisting of: R^{a} and R^{b},
R¹ is selected from the group consisting of:
(a) C(O)OH,
(b) C(O)NR^{d}R^{e}, and
(c) C(O)OC₁₋₆ alkyl, wherein the C₁₋₆ alkyl is optionally substituted with 1-3 R^{c};
each R², R³, R⁴, and R⁵ is independently selected from the group consisting of: halo, CN, C₁₋₃ alkyl, C₁₋₃ haloalkyl, C₁₋₃ alkoxy, C₁₋₃ haloalkoxy, OH, and NR^{d}R^{e}
m2 is 0, 1, or 2;
m3 and m4 are independently 0, 1, 2, or 3;
m5 is 0, 1, 2, 3, or 4;
L is -(L^{A})ₙ₁-, wherein L^{A} and n1 are defined according to (AA) or (BB):
   (AA)
n1 is an integer from 3 to 15; and
each L^{A} is independently selected from the group consisting of: L^{A1} L^{A3}, and L^{A4}, provided that 1-3 occurrences of L^{A} is L^{A4};
   (BB)
n1 is an integer from 0 to 20; and
each L^{A} is independently selected from the group consisting of: L^{A1} and L^{A3};
each L^{A1} is independently selected from the group consisting of: —CH₂—, —CHR^{L}—, and —C(R^{L})₂—;
each L^{A3} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b};
provided that L does not contain any N—O, O—O, N—N, N—S(O)o, or O—S(O)₀₋₂ bonds;
wherein each R^{L} is independently selected from the group consisting of: halo; cyano; —OH; —C₁₋₆ alkoxy; —C₁₋₆ haloalkoxy; —NR^{d}R^{e}; C(═O)N(R′)₂; S(O)₀₋₂(C₁₋₆ alkyl); S(O)₀₋₂(C₁₋₆ haloalkyl); S(O)₁₋₂N(R^{f})₂; —R¹; and C₁₋₆ alkyl optionally substituted with 1-6 R^{c};
Ring C is selected from the group consisting of:

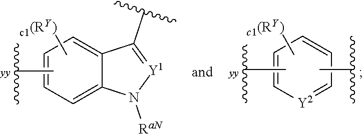

c1 is 0, 1, 2, or 3;
each R^{Y} is independently selected from the group consisting of R^{a} and R^{b};
R^{aN} is H or C₁₋₆ alkyl optionally substituted with 1-3 R^{c};
Y¹ and Y² are independently N, CH, or CR^{Y};
yy represents the point of attachment to L;
X is CH, C, or N;
the [symbol] is a single bond or a double bond;
L^{C} is selected from the group consisting of: a bond, —CH₂—, —CHR^{a}—, —C(R^{a})₂—, —N(R^{d})—, and O, provided that when X is N, then L^{C} is other than O;
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R)₂; and
(q) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
each b is independently 1, 2, or 3;
each -L^{h} is independently selected from the group consisting of: —O—; —N(H)—; —N(C₁₋₃ alkyl)-; —S(O)₀₋₂—; C(═O); and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(Rr)₂; S(O)₁₋₂(C₁₋₆ alkyl); S(O)₁₋₂(C₁₋₆ haloalkyl); S(O)₁₋₂N(R^{f})₂; and C₁₋₆ alkyl optionally substituted with 1-3 R^{h}.
each R^{f} is independently selected from the group consisting of: H and C₁₋₆ alkyl optionally substituted with 1-3 R^{h};
each R^{g} is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂.

Embodiment 2. The compound of Embodiment 1, wherein Ring A is a phenylene optionally substituted with 1-3 R^{a}.

Embodiment 3. The compound of Embodiment 1 or 2, wherein Ring A is

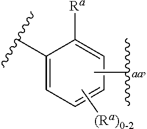

wherein aa represents the point of attachment to L.

Embodiment 4. The compound of any one of Embodiments 1-3, wherein Ring A is

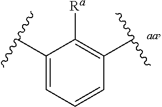

wherein aa represents the point of attachment to L.

Embodiment 5. The compound of any one of Embodiments 1-3, wherein Ring A is

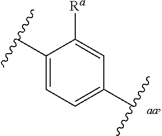

wherein aa represents the point of attachment to L.

Embodiment 6. The compound of any one of Embodiments 1-5, wherein one R^{a} present on Ring A is C₁₋₃ alkyl optionally substituted with 1-3 F.

Embodiment 7. The compound of any one of Embodiments 1-6, wherein one R^{a} present on Ring A is methyl or CF₃.

Embodiment 8. The compound of any one of Embodiments 1-7, wherein R¹ is C(O)OH.

Embodiment 9. The compound of any one of Embodiments 1-8, wherein m2 is 0.

Embodiment 10. The compound of any one of Embodiments 1-9, wherein m3 is 0.

Embodiment 11. The compound of any one of Embodiments 1-10, wherein m4 is 0.

Embodiment 12. The compound of any one of Embodiments 1-11, wherein m5 is 0.

Embodiment 13. The compound of any one of Embodiments 1-8, wherein m2 is 0; m3 is 0; m4 is 0; and m5 is 0.

Embodiment 14. The compound of any one of Embodiments 1-13, wherein Ring C is

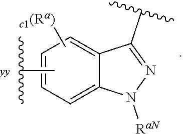

Embodiment 15. The compound of any one of Embodiments 1-14, wherein Ring C is

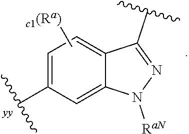

Embodiment 16. The compound of any one of Embodiments 1-14, wherein Ring C is

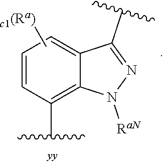

Embodiment 17. The compound of any one of Embodiments 14-16, wherein c1 is 0.

Embodiment 18. The compound of any one of Embodiments 14-17, wherein R^{aN} is C₁₋₃ alkyl.

Embodiment 19. The compound of any one of Embodiments 14-18, wherein R^{aN} is methyl.

Embodiment 20. The compound of any one of Embodiments 14-19, wherein L^{C} is a bond.

Embodiment 21. The compound of any one of Embodiments 14-20, wherein X is CH.

Embodiment 22. The compound of any one of Embodiments 1-14, wherein the

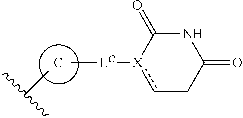

moiety is

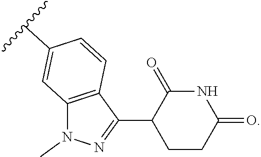 [SMILES: *c1ccc2c(C3CCC(=O)NC3=O)nn(C)c2c1]

Embodiment 23. The compound of any one of Embodiments 1-14, wherein the

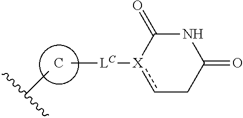

moiety is

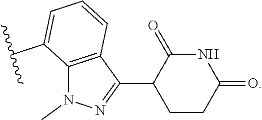 [SMILES: *c1cccc2c(C3CCC(=O)NC3=O)nn(C)c12]

Embodiment 24. The compound of any one of Embodiments 1-23, wherein L is -(L^{A})ₙ₁-; and L^{A} and n1 are defined according to (AA).

Embodiment 25. The compound of any one of Embodiments 1-24, wherein n1 is an integer from 3 to 5.

Embodiment 26. The compound of any one of Embodiments 1-24, wherein n1 is an integer from 5 to 9.

Embodiment 27. The compound of any one of Embodiments 1-24 or 26, wherein n1 is 6, 7, or 8.

Embodiment 28. The compound of any one of Embodiments 1-24, wherein n1 is an integer from 9 to 12.

Embodiment 29. The compound of any one of Embodiments 1-28, wherein 1-2 occurrences of L^{A} is L^{A4}.

Embodiment 30. The compound of any one of Embodiments 1-28, wherein one occurrence of L^{A} is L^{A4}.

Embodiment 31. The compound of any one of Embodiments 1-28, wherein two occurrences of L^{A} are L^{A4}.

Embodiment 32. The compound of any one of Embodiments 1-31, wherein each L^{A4} is independently selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

Embodiment 33. The compound of any one of Embodiments 1-32, wherein 1-4 occurrences of L^{A} is L^{A3}.

Embodiment 34. The compound of any one of Embodiments 1-33, wherein 1-3 occurrences of L^{A} is L^{A3}.

Embodiment 35. The compound of any one of Embodiments 1-34, wherein 0-1 occurrence of L^{A3} is C(═O); and each remaining occurrence of L^{A3} is independently selected from the group consisting of: —O—; —N(H)—; and —N(C₁₋₃ alkyl)-.

Embodiment 36. The compound of any one of Embodiments 1-35, wherein 2-7 occurrences of L^{A} is L^{A1}.

Embodiment 37. The compound of any one of Embodiments 1-36, wherein 2-5 occurrences of L^{A} is L^{A1}.

Embodiment 38. The compound of any one of Embodiments 1-37, wherein 0-2 occurrences of L^{A1} is —CHR^{L}— or —C(R^{L})₂—; and each remaining occurrence of L^{A1} is —CH₂—.

Embodiment 39. The compound of any one of Embodiments 1-38, wherein each occurrence of L^{A1} is —CH₂—.

Embodiment 40. The compound of any one of Embodiments 1-38, wherein one occurrence of L^{A1} is —CHR^{L}— or —C(R^{L})₂—; and each remaining occurrence of L^{A1} is —CH₂—.

Embodiment 41. The compound of any one of Embodiments 1-40, wherein each R^{L} is independently selected from the group consisting of: —F and —C₁₋₃ alkyl optionally substituted with 1-3 F.

Embodiment 42. The compound of any one of Embodiments 1-24, wherein L is -(L^{A})ₙ₁-, and L^{A} and n1 are defined according to (AA):
n1 is an integer from 5 to 9;
1-2 occurrences of L^{A} is L^{A4};
2-7 occurrences of L^{A} are L^{A1}; and
1-4 occurrences of L^{A} is L^{A3}.

Embodiment 43. A compound selected from the group consisting of 101, 102, 103, 104, 105, 106, 107, 108, 109, 110, 111, 112, 113, 114, 115, 116, 116a, 117, 117a, 118, 119, 120, 121, 122, 123, 124, 124a, 125, 125a, 126, 127, 128, 129, 130, 131, 131a, 132, 133, 134, 135, 136, 137, 138, 138a, 138b, 139, 140, 141, 142, 143, 144, 145, 146, 146a, 146b, 147, 147a, 147b, 148, 148a, 148b, 149, 150, 151, 152, 153, 154, 154a, 155, 156, 157, 158, 159, 159a, 160, 160a, 160b, 161, 161a, 162, 162a, 162b, 163, 164, 164a, 164b, 165, 166, 167, 167a, 167b, 168, 168a, 169, 169a, 169b, 170, 171, 171a, 172, 173, 173a, 173b, 174, 174a, 174b, 175, 176, 176a, 177, 177a, 178, 179, 179a, 180, 180a, 180b, 181, 182, 182a, 183, 183a, 184, 185, 185a, 186, 186a, 186b, 187, 187a, 187b, 188, 189, 190, 191, 192, 193, 193a, 193b, 194, 195, 196, 197, 197a, 197b, 198, 199, 200, 201, 202, 202a, 202b, 203, 204, 204a, 205, 206, 207, 207a, 207b, 208, 209, 209a, 210, 210a, 210b, 211, 211a, 212, 212a, 213, 214, 214a, 215, 215a, 216, 216a, 216b, 217, 218, 218a, 218b, 219, 219a, 219b, 220, 221, 222, 223, 224, 224a, 224b, 225, 226, 227, 227a, 228, 229, 230, 231, 231a, 231b, 232, 233, 234, 235, 236, 237, 238, 239, 239a, 240, 240a, 240b, 241, 242, 243, 244, 245, 245a, 245b, 246, 246a, 246b, 247, 248, 249, 250, 250a, 251, 251a, 252, 252a, 253, 253a, 254, 254a, 255, 256, 257, 258, 259, 259a, 260, 261, 262, 262a, 262b, 263, 263a, 263b, 264, 265, 266, 266a, 266b, 267, 268, 268a, 268b, 269, 269a, 270, 270a, 271, 271a, 271b, 272, 272a, 272b, 273, 273a, 273b, 274, 274a, 274b, 275, 275a, 275b, 276, 276a, 276b, 277, 278, 278a, 278b, 279, 280, 281, 281a, 281b, 282, 282a, 282b, 283, 283a, 283b, 284, 284a, 284b, 285, 286, 287, 288, 289, 289a, 289b, 290, 291, 292, 293, 293a, 294, 295, 296, 296a, 297, 297a, 297b, 298, 298a, 299, 299a, 300, 300a, 300b, 301, 301a, 301b, 302, 303, 304, 305, 306, 307, 307a, 307b, 308, 308a, 308b, 309, 310, 310a, 310b, 311, 311a, 311b, 312, 313, 314, 316, 316a, 317, 318, 318a, 319, 319a, 320, 320a, 320b, 321, 321a, 321b, 322, 322a, 323, 323a, 324, 324a, 325, 325a, 326, 326a, 327, 327a, 328, 328a, 329, 329a, 330, 330a, 331, 331a, 331b, 332, 332a, 332b, 333, 333a, 333b, 334, 334a, 335, 335a, 336, 336a, 337, 337a, 338, 338a, 339, 339a, 339b, 340, 340a, 341, 341a, 342, 342a, 343, 343a, 343b, 343c, 343d, 344, 344a, 344b, 344c, 344d, 345, 345a, 346, 346a, 346b, 347, 347a, 347b, 349, 349a, 349b, 350, 350a, 351, 351a, 351b, 352, 352a, 352b, 353, 353a, 353b, 354, 354a, 354b, 355, 356, 357, as depicted in Table C1, or a pharmaceutically acceptable salt thereof, or
the compound is selected from the group consisting of 101, 102, 103, 104, 105, 106, 107, 108, 109, 110, 111, 112, 113, 114, 115, 116, 116a, 117, 117a, 118, 119, 120, 121, 122, 123, 124, 124a, 125, 125a, 126, 127, 128, 129, 130, 131, 131a, 132, 133, 134, 135, 136, 137, 138, 138a, 138b, 139, 140, 141, 142, 143, 144, 145, 146, 146a, 146b, 147, 147a, 147b, 148, 148a, 148b, 149, 150, 151, 152, 153, 154, 154a, 155, 156, 157, 158, 159, 159a, 160, 160a, 160b, 161, 161a, 162, 162a, 162b, 163, 164, 164a, 164b, 165, 166, 167, 167a, 167b, 168, 168a, 169, 169a, 169b, 170, 171, 171a, 172, 173, 173a, 173b, 174, 174a, 174b, 175, 176, 176a, 177, 177a, 178, 179, 179a, 180, 180a, 180b, 181, 182, 182a, 183, 183a, 184, 185, 185a, 186, 186a, 187, 187a, 187b, 188, 189, 190, 191, 192, 193, 193a, 193b, 194, 195, 196, 197, 197a, 197b, 198, 199, 200, 201, 202, 202a, 202b, 203, 204, 204a, 205, 206, 207, 207a, 207b, 208, 209, 209a, 210, 210a, 211, 211a, 212, 212a, 213, 214, 214a, 215, 215a, 216, 216a, 216b, 217, 218, 218a, 219, 219a, 219b, 220, 221, 222, 223, 224, 224a, 224b, 225, 226, 227, 227a, 228, 229, 230, 231, 231a, 231b, 232, 233, 234, 235, 236, 237, 238, 239, 239a, 240, 240a, 240b, 241, 242, 243, 244, 245, 245a, 245b, 246, 246a, 246b, 247, 248, 249, 250, 250a, 251, 251a, 252, 252a, 253, 253a, 254, 254a, 255, 256, 257, 258, 259, 259a, 260, 261, 262, 262a, 262b, 263, 263a, 263b, 264, 265, 266, 266a, 266b, 267, 268, 268a, 268b, 269, 269a, 270, 270a, 271, 271a, 271b, 272, 272a, 272b, 273, 273a, 273b, 274, 274a, 274b, 275, 275a, 276, 276a, 276b, 277, 278, 278a, 278b, 279, 280, 281, 281a, 281b, 282, 282a, 282b, 283, 283a, 283b, 284, 284a, 284b, 285, 286, 287, 288, 289, 289a, 289b, 290, 291, 292, 293, 293a, 294, 295, 296, 296a, 297, 297a, 297b, 298, 298a, 299, 299a, 300, 300a, 300b, 301, 301a, 301b, 302, 303, 304, 305, 306, 307, 307a, 307b, 308, 308a, 308b, 309, 310, 310a, 310b, 311, 311a, 312, 313, 314, 316, 316a, 317, 318, 318a, 319, 319a, 320, 320a, 320b, 321, 321a, 321b, 322, 322a, 323, 323a, 324, 324a, 325, 325a, 326, 326a, 327, 327a, 328, 328a, 329, 329a, 330, 330a, 331, 331a, 331b, 332, 332a, 332b, 333, 333a, 334, 334a, 335, 335a, 336, 336a, 337, 337a, 338, 338a, 339, 339a, 340, 340a, 341, 341a, 342, 342a, 343, 343a, 343b, 343c, 343d, 344, 344a, 344b, 344c, 344d, 345, 345a, 346, 346a, 346b, 347, 347a, 347b, 349, 349a, 349b, 350, 350a, 351, 351a, 351b, 352, 352a, and 352b as depicted in Table C1, or a pharmaceutically acceptable salt thereof, or
the compound is selected from the group consisting of 101, 102, 103, 104, 105, 106, 107, 108, 109, 110, 111, 112, 113, 114, 115, 116, 116a, 117, 117a, 118, 119, 120, 121, 122, 123, 124, 124a, 125, 125a, 126, 127, 128, 129, 130, 131, 131a, 132, 133, 134, 135, 136, 137, 138, 138a, 138b, 139, 140, 141, 142, 143, 144, 145, 146, 146a, 146b, 147, 147a, 147b, 148, 148a, 148b, 149, 150, 151, 152, 153, 154, 154a, 155, 156, 157, 158, 159, 159a, 160, 160a, 160b, 161, 161a, 162, 162a, 162b, 163, 164, 164a, 164b, 165, 166, 167, 167a, 167b, 168, 168a, 169, 169a, 169b, 170, 171, 171a, 172, 173, 173a, 173b, 174, 174a, 174b, 175, 176, 176a, 177, 177a, 178, 179, 179a, 180, 180a, 180b, 181, 182, 182a, 183, 183a, 184, 185, 185a, 186, 186a, 187, 187a, 187b, 188, 189, 190, 191, 192, 193, 193a, 193b, 194, 195, 196, 197, 197a, 197b, 198, 199, 200, 201, 202, 202a, 202b, 203, 204, 204a, 205, 206, 207, 207a, 207b, 208, 209, 209a, 210, 210a, 211, 211a, 212, 212a, 213, 214, 214a, 215, 215a, 216, 216a, 216b, 217, 218, 218a, 219, 219a, 219b, 220, 221, 222, 223, 224, 224a, 224b, 225, 226, 227, 227a, 228, 229, 230, 231, 231a, 231b, 232, 233, 234, 235, 236, 237, 238, 239, 239a, 240, 240a, 240b, 241, 242, 243, 244, 245, 245a, 245b, 246, 246a, 246b, 247, 248, 249, 250, 250a, 251, 251a, 252, 252a, 253, 253a, 254, 254a, 255, 256, 257, 258, 259, 259a, 260, 261, 262, 262a, 262b, 263, 263a, 263b, 264, 265, 266, 266a, 266b, 267, 268, 268a, 268b, 269, 269a, 270, 270a, 271, 271a, 272, 272a, 272b, 273, 273a, 273b, 274, 274a, 274b, 275, 275a, 276, 276a, 276b, 277, 278, 278a, 278b, 279, 280, 281, 281a, 281b, 282, 282a, 282b, 283, 283a, 283b, 284, 284a, 284b, 285, 286, 287, 288, 289, 289a, 289b, 290, 291, 292, 293, 293a, 294, 295, 296, 296a, 297, 297a, 297b, 298, 298a, 299, 299a, 300, 300a, 300b, 301, 301a, 301b, 302, 303, 304, 305, 306, 307, 307a, 307b, 308, 308a, 308b, 309, 310, 310a, 310b, 311, 311a, 312, 313, 314, 315, 315a, 316, 316a, 317, 318, 318a, 319, 319a, 320, 320a, 320b, 321, 321a, 321b, 322, 322a, 323, 323a, 324, 324a, 325, 325a, 326, 326a, 327, 327a, 328, 328a, 329, 329a, 330, 330a, 331, 331a, 331b, 332, 332a, 332b, 333, 333a as depicted in Table C1, or a pharmaceutically acceptable salt thereof, or
the compound is selected from the group consisting of 101, 102, 103, 104, 105, 106, 107, 108, 109, 110, 111, 112, 113, 114, 115, 116, 116a, 117, 117a, 118, 119, 120, 121, 122, 123, 124, 124a, 125, 125a, 126, 127, 128, 129, 130, 131, 131a, 132, 133, 134, 135, 136, 137, 138, 138a, 138b, 139, 140, 141, 142, 143, 144, 145, 146, 146a, 146b, 147, 147a, 147b, 148, 148a, 148b, 149, 150, 151, 152, 153, 154, 154a, 155, 156, 157, 158, 159, 159a, 160, 160a, 160b, 161, 161a, 162, 162a, 162b, 163, 164, 164a, 164b, 165, 166, 167, 167a, 167b, 168, 168a, 169, 169a, 169b, 170, 171, 171a, 172, 173, 173a, 173b, 174, 174a, 174b, 175, 176, 176a, 177, 177a, 178, 179, 179a, 180, 180a, 180b, 181, 182, 182a, 183, 183a, 184, 185, 185a, 186, 186a, 187, 187a, 187b, 188, 189, 190, 191, 192, 193, 193a, 193b, 194, 195, 196, 197, 197a, 197b, 198, 199, 200, 201, 202, 202a, 202b, 203, 204, 204a, 205, 206, 207, 207a, 207b, 208, 209, 209a, 210, 210a, 211, 211a, 212, 212a, 213, 214, 214a, 215, 215a, 216, 216a, 216b, 217, 218, 218a, 219, 219a, 219b, 220, 221, 222, 223, 224, 224a, 224b, 225, 226, 227, 227a, 228, 229, 230, 231, 231a, 231b, 232, 233, 234, 235, 236, 237, 238, 239, 239a, 240, 240a, 240b, 241, 242, 243, 244, 245, 245a, 245b, 246, 246a, 246b, 247, 248, 249, 250, 250a, 251, 251a, 252, 252a, 253, 253a, 254, 254a, 255, 256, 257, 258, 259, 259a, 260, 261, 262, 262a, 262b, 263, 263a, 263b, 264, 265, 266, 266a, 266b, 267, 268, 268a, 268b, 269, 269a, 270, 270a, 271, 271a, 272, 272a, 272b, 273, 273a, 273b, 274, 274a, 274b, 275, 275a, 276, 276a, 276b, 277, 278, 278a, 278b, 279, 280, 281, 281a, 281b, 282, 282a, 282b, 283, 283a, 283b, 284, 284a, 284b, 285, 286, 287, 288, 289, 289a, 289b, 290, 291, 292, 293, 293a, 294, 295, 296, 296a, 297, 297a, 297b, 298, 298a, 299, 299a, 300, 300a, 300b, 301, 301a, 301b, 302, 303, 304, 305, 306, 307, 307a, 307b, 308, 308a, 308b, 309, 310, 310a, 311, 311a, 312, 313, 314, 315, 315a, 316, 316a, 317, 318, 318a, 319, 319a, 320, 320a, 320b, 321, 321a, 321b, 322, 322a, 323, 323a, 324, 324a, 325, 325a, 326, 326a, 327, 327a as depicted in Table C1, or a pharmaceutically acceptable salt thereof; or
the compound is selected from the group consisting of 101, 102, 103, 104, 105, 106, 107, 108, 109, 110, 111, 112, 113, 114, 115, 116, 116a, 117, 117a, 118, 119, 120, 121, 122, 123, 124, 124a, 125, 125a, 126, 127, 128, 129, 130, 131, 131a, 132, 133, 134, 135, 136, 137, 138, 138a, 138b, 139, 140, 141, 142, 143, 144, 145, 146, 146a, 146b, 147, 147a, 147b, 148, 148a, 148b, 149, 150, 151, 152, 153, 154, 154a, 155, 156, 157, 158, 159, 159a, 160, 160a, 160b, 161, 161a, 162, 162a, 162b, 163, 164, 164a, 164b, 165, 166, 167, 167a, 167b, 168, 168a, 169, 169a, 169b, 170, 171, 171a, 172, 173, 173a, 173b, 174, 174a, 174b, 175, 176, 176a, 177, 177a, 178, 179, 179a, 180, 180a, 180b, 181, 182, 182a, 183, 183a, 184, 185, 185a, 186, 186a, 187, 187a, 187b, 188, 189, 190, 191, 192, 193, 193a, 193b, 194, 195, 196, 197, 197a, 197b, 198, 199, 200, 201, 202, 202a, 202b, 203, 204, 204a, 205, 206, 207, 207a, 207b, 208, 209, 209a, 210, 210a, 211, 211a, 212, 212a, 213, 214, 214a, 215, 215a, 216, 216a, 216b, 217, 218, 218a, 219, 219a, 219b, 220, 221, 222, 223, 224, 224a, 224b, 225, 226, 227, 227a, 228, 229, 230, 231, 231a, 231b, 232, 233, 234, 235, 236, 237, 238, 239, 239a, 240, 240a, 240b, 241, 242, 243, 244, 245, 245a, 245b, 246, 246a, 246b, 247, 248, 249, 250, 250a, 251, 251a, 252, 252a, 253, 253a, 254, 254a, 255, 256, 257, 258, 259, 259a, 260, 261, 262, 262a, 262b, 263, 263a, 263b, 264, 265, 266, 266a, 266b, 267, 268, 268a, 268b, 269, 269a, 270, 270a, 271, 271a, 272, 272a, 272b, 273, 273a, 273b, 274, 274a, 274b, 275, 275a, 276, 276a, 276b, 277, 278, 278a, 278b, 279, 280, 281, 281a, 281b, 282, 282a, 282b, 283, 283a, 283b, 284, 284a, 284b, 285, 286, 287, 288, 289, 289a, 289b, 290, 291 as depicted in Table C1, or a pharmaceutically acceptable salt thereof; or
the compound is selected from the group consisting of 101, 102, 103, 104, 105, 106, 107, 108, 109, 110, 111, 112, 113, 114, 115, 116, 116a, 117, 117a, 118, 119, 120, 121, 122, 123, 124, 124a, 125, 125a, 126, 127, 128, 129, 130, 131, 131a, 132, 133, 134, 135, 136, 137, 138, 138a, 139, 140, 141, 142, 143, 144, 145, 146, 146a, 147, 147a, 148, 148a, 149, 150, 151, 152, 153, 154, 154a, 155, 156, 157, 158, 159, 159a, 160, 160a, 160b, 161, 161a, 162, 162a, 162b, 163, 164, 164a, 164b, 165, 166, 167, 167a, 167b, 168, 168a, 169, 169a, 169b, 170, 171, 171a, 172, 173, 173a, 173b, 174, 174a, 174b, 175, 176, 176a, 177, 177a, 178, 179, 179a, 180, 180a, 180b, 181, 182, 182a, 183, 183a, 184, 185, 185a, 186, 186a, 187, 187a, 187b, 188, 189, 190, 191, 192, 193, 193a, 193b, 194, 195, 196, 197, 197a, 197b, 198, 199, 200, 201, 202, 202a, 202b, 203, 204, 204a, 205, 206, 207, 207a, 207b, 208, 209, 209a, 210, 210a, 211, 211a, 212, 212a, 213, 214, 214a, 215, 215a, 216, 216a, 216b, 217, 218, 218a, 219, 219a, 219b, 220, 221, 222, 223, 224, 224a, 224b, 225, 226, 227, 227a, 228, 229, 230, 231, 231a, 231b, 232, 233, 234, 235, 236, 237, 238, 239, 239a, 240, 240a, 240b, and 241 as depicted in Table C1, or a pharmaceutically acceptable salt thereof; or
the compound is selected from the group consisting of 101, 102, 103, 104, 105, 106, 107, 108, 109, 110, 111, 112, 113, 114, 115, 116, 116a, 117, 117a, 118, 119, 120, 121, 122, 123, 124, 124a, 125, 125a, 126, 127, 128, 129, 130, 131, 131a, 132, 133, 134, 135, 136, 137, 138, 138a, 139, 140, 141, 142, 143, 144, 145, 146, 146a, 147, 147a, 148, 148a, 149, 150, 151, 152, 153, 154, 154a, 155, and 156, as depicted in Table C1, or a pharmaceutically acceptable salt thereof.

Embodiment 44. The compound of Embodiment 43, wherein the compound does not exhibit Yₘᵢₙ (6 h)≥70% and/or Yₘᵢₙ (24 h)≥70% under conditions described in Example B1.

Embodiment 45. A pharmaceutical composition comprising a compound of any one of Embodiments 1-44, or a pharmaceutically acceptable salt thereof, and a pharmaceutically acceptable excipient.

Embodiment 46. A compound of any one of Embodiments 1-44, or a pharmaceutically acceptable salt thereof, or the pharmaceutical composition of Embodiment 45 for use in treatment.

Embodiment 47. A compound of any one of Embodiments 1-44 or a pharmaceutically acceptable salt thereof, or the pharmaceutical composition of Embodiment 45 for use in the treatment of a cancer selected from the group consisting of essential thrombocythemia, polycythemia vera, myelofibrosis, and a combination thereof.

Embodiment 48. The compound or pharmaceutical composition of Embodiment 47 and an additional therapeutic agent or therapy selected from the group consisting of fedratinib (e.g., fedratinib dihydrochloride monohydrate), momelotinib (e.g., momelotinib dihydrochloride), pacritinib (e.g., pacritinib citrate), ruxolitinib (e.g., ruxolitinib phosphate), and a combination thereof for use in treatment.

Embodiment 49. The compound or pharmaceutical composition of Embodiment 48 and an additional therapeutic agent or therapy selected from the group consisting of fedratinib (e.g., fedratinib dihydrochloride monohydrate), momelotinib (e.g., momelotinib dihydrochloride), pacritinib (e.g., pacritinib citrate), ruxolitinib (e.g., ruxolitinib phosphate), and a combination thereof for use in treatment of a cancer selected from the group consisting of essential thrombocythemia, polycythemia vera, myelofibrosis, and a combination thereof.

Embodiment 50. A compound of any one of Embodiments 1-44, or a pharmaceutically acceptable salt thereof, or the pharmaceutical composition of Embodiment 45 for use in a method of treating cancer selected from the group consisting of essential thrombocythemia, polycythemia vera, myelofibrosis, and a combination thereof, the method comprising administering to a subject a therapeutically effective amount of a compound of Embodiments 1-44, or a pharmaceutically acceptable salt thereof, or the pharmaceutical composition of Embodiment 45.

Embodiment 51. The compound or pharmaceutical composition of Embodiment 50, wherein the method comprises administering to the subject a therapeutically effective amount of an additional therapeutic agent or therapy selected from the group consisting of fedratinib (e.g., fedratinib dihydrochloride monohydrate), momelotinib (e.g., momelotinib dihydrochloride), pacritinib (e.g., pacritinib citrate), ruxolitinib (e.g., ruxolitinib phosphate), and a combination thereof.

Embodiment 52. The compound or pharmaceutical composition of Embodiment 54, wherein the compound of any one of Embodiments 1-44, or a pharmaceutically acceptable salt thereof, or the pharmaceutical composition of Embodiment 45, and the additional therapeutic agent or therapy are administered simultaneously, separately, or sequentially.

1. A compound of Formula (I): 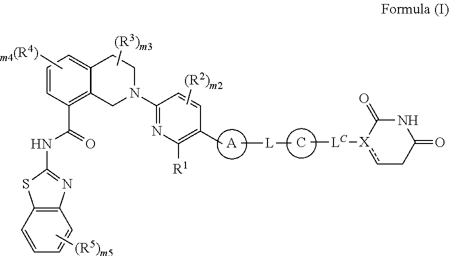
or a pharmaceutically acceptable salt thereof, wherein:
Ring A is selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) phenylene or 5-6 membered heteroarylene, each of which is optionally substituted with 1-3 substituents independently selected from the group consisting of: R^{a} and R^{b},
R¹ is selected from the group consisting of:
(a) C(O)OH,
(b) C(O)NR^{d}R^{e}, and
(c) C(O)OC₁₋₆ alkyl, wherein the C₁₋₆ alkyl is optionally substituted with 1-3 R^{e};
each R², R³, R⁴, and R⁵ is independently selected from the group consisting of: halo, CN, C₁₋₃ alkyl, C₁₋₃ haloalkyl, C₁₋₃ alkoxy, C₁₋₃ haloalkoxy, OH, and NR^{d}R^{e};
m2 is 0, 1, or 2;
m3 and m4 are independently 0, 1, 2, or 3;
m5 is 0, 1, 2, 3, or 4;
L is -(L^{A})ₙ₁-, wherein L^{A} and n1 are defined according to (AA) or (BB):
(AA)
n1 is an integer from 3 to 15; and
each L^{A} is independently selected from the group consisting of: L^{A1} L^{A3}, and L^{A4} provided that 1-3 occurrences of L^{A} is L^{A4};
(BB)
n1 is an integer from 0 to 20; and
each L^{A} is independently selected from the group consisting of: L^{A1} and L^{A3};
each L^{A1} is independently selected from the group consisting of: —CH₂—, —CHR^{L}—, and —C(R^{L})₂—;
each L^{A} is independently selected from the group consisting of: —N(R^{d})—, —N(R^{b})—, —O—, —S(O)₀₋₂—, and C(═O);
each L^{A4} is independently selected from the group consisting of:
(a) C₃₋₁₅ cycloalkylene or 3-15 membered heterocyclylene, each of which is optionally substituted with 1-6 substituents independently selected from the group consisting of: R^{a} and R^{b}; and
(b) C₆₋₁₅ arylene or 5-15 membered heteroarylene, each of which is optionally substituted with 1-6 substituents independently selected from the group consisting of: R^{a} and R^{b},
provided that L does not contain any N—O, 0-0, N—N, N—S(O)o, or O—S(0)₀₋₂ bonds;
wherein each R^{L} is independently selected from the group consisting of: halo; cyano; —OH; —C₁₋₆ alkoxy; —C₁₋₆ haloalkoxy; —NR^{d}R^{e}; C(═O)N(R)₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{c};
Ring C is selected from the group consisting of: 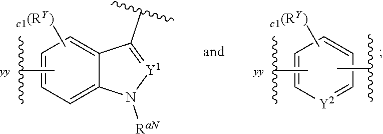
c1 is 0, 1, 2, or 3;
each R^{Y} is independently selected from the group consisting of R^{a} and R^{b},
R^{aN} is H or C₁₋₆ alkyl optionally substituted with 1-3 R^{c};
Y¹ and Y² are independently N, CH, or CR^{Y};
yy represents the point of attachment to L;
X is CH, C, or N;
the [symbol] is a single bond or a double bond;
L^{C} is selected from the group consisting of: a bond, —CH₂—, —CHR^{a}—, —C(R^{a})₂—, —N(R^{d})—, and O, provided that when X is N, then L^{C} is other than O;
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(i) C(═O)OH;
(k) C(═O)OC₁₋₆ alkyl;
(l) C(═O)OC₁₋₆ haloalkyl;
(m) C(═O)N(R^{f});
(n) S(O)₀₋₂(C₁₋₆ alkyl);
(o) S(O)₀₋₂(C₁₋₆ haloalkyl);
(p) S(O)₁₋₂N(R′)₂; and
(q) C₁₋₆ alkyl, C₂₋₆ alkenyl, or C₂₋₆ alkynyl, each optionally substituted with 1-6 R^{e};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—; —N(H)—; —N(C₁₋₃ alkyl)-; —S(O)₀₋₂—; C(═O); and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{e} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl;
C(═O)C₁₋₆ haloalkyl; C(═O)OC₁₋₆ alkyl; C(═O)OC₁₋₆ haloalkyl; C(═O)N(R)₂; S(O)₁₋₂(C₁₋₆ alkyl); S(O)₁₋₂(C₁₋₆ haloalkyl); S(O)₁₋₂N(R′)₂; and C₁₋₆ alkyl optionally substituted with 1-3 R^{h},
each R^{f} is independently selected from the group consisting of: H and C₁₋₆ alkyl optionally substituted with 1-3 R^{h};
each R^{g} is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂.

2. The compound of claim 1, wherein Ring A is a phenylene optionally substituted with 1-3 R^{a}.

3. The compound of claim 1, wherein Ring A is 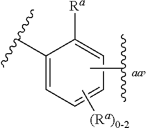
wherein aa represents the point of attachment to L.

4. (canceled)

5. The compound of claim 3, wherein one R^{a} present on Ring A is C₁₋₃ alkyl optionally substituted with 1-3 F.

6. The compound of claim 1, wherein R¹ is C(O)OH.

7.-10. (canceled)

11. The compound of claim 1, wherein m2 is 0; m3 is 0, m4 is 0; and m5 is 0.

12. The compound of m claim 1, wherein Ring C is

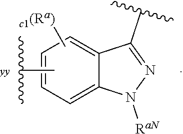

13. The compound of claim 12, wherein c1 is 0.

14. The compound of claim 12, wherein R^{aN} is C₁₋₃ alkyl.

15. The compound of claim 12, wherein L^{C} is a bond.

16. The compound of claim 12, wherein X is CH.

17. The compound of claim 1, wherein the 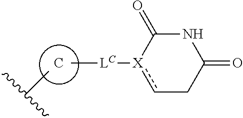
moiety is 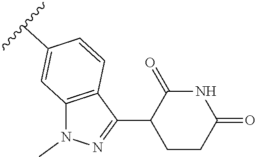 [SMILES: *c1ccc2c(C3CCC(=O)NC3=O)nn(C)c2c1]
or

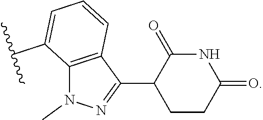 [SMILES: *c1cccc2c(C3CCC(=O)NC3=O)nn(C)c12]

18. The compound of claim 1, wherein L is -(L^{A})ₙ₁-; and L^{A} and n1 are defined according to (AA).

19. The compound of claim 18, wherein n1 is an integer from 3 to 5; or n1 is an integer from 5 to 9; or n1 is 6, 7, or 8; or n1 is an integer from 9 to 12.

20. The compound of claim 18, wherein 1-2 occurrences of L^{A} is L^{A4}.

21. The compound of claim 20, wherein each L^{A4} is independently selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

22. The compound of claim 18, wherein 1-4 occurrences of L^{A} is L^{A3}.

23. (canceled)

24. The compound of claim 18, wherein 2-7 occurrences of L^{A} is L^{A1}.

25. The compound of claim 24, wherein 0-2 occurrences of L^{A1} is —CHR^{L}— or —C(R^{L})₂—; and each remaining occurrence of L^{A1} is —CH₂—.

26. (canceled)

27. The compound of claim 25, wherein each R^{L} is independently selected from the group consisting of: —F and —C₁₋₃ alkyl optionally substituted with 1-3 F.

28. The compound of claim 1, wherein L is -(L^{A})ₙ₁-, and L^{A} and n1 are defined according to (AA):
n1 is an integer from 5 to 9;
1-2 occurrences of L^{A} is L^{A4};
2-7 occurrences of L^{A} are L^{A1}; and
1-3 occurrences of L^{A} is L^{A3}.

29. The compound of claim 1, wherein L is selected from the group consisting of:
(i) -(L^{A3})₀₋₂-(L^{A1})₀₋₅-L^{A4}-(L^{A1})₀₋₅-(L^{A3})₁₋₂-_{bb};
(ii) -(L^{A3})₀₋₂-(L^{A1})₀₋₅-L^{A4}-(L^{A1})₀₋₅-L^{A4}-_{bb};
(iii) -(L^{A3})₀₋₂-(L^{A1})₀₋₅-L^{A4}-(L^{A1})₀₋₅-L^{A3}-L^{A4}-_{bb}; and
(iv) -(L^{A3})₀₋₁-(L^{A1})₂₋₃-L^{A3}-L^{A4}-(L^{A1})₀₋₅-(L^{A3})₁₋₂-_{bb};
provided that L contains 1-7 L^{A1}; and wherein bb represents the point of attachment to Ring C.

30. The compound of claim 1, wherein L is:
-(L^{A3})₀₋₂-(L^{A1})₂₋₉-(L^{A3})₀₋₁-L^{A4}-_{bb},
wherein bb represents the point of attachment to Ring C.

31. The compound of claim 29, wherein each L^{A4} is independently selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}.

32. The compound of claim 29, wherein 0-1 occurrence of L^{A1} is —CHR^{L}— or —C(R^{L})₂—; and each remaining occurrence of L^{A1} is —CH₂—.

33. (canceled)

34. The compound of claim 1, wherein L is:
-(L^{A3})₀₋₂-(L^{A1})₀₋₅-L^{A4}-(L^{A1})₀₋₅-(L^{A3})₁₋₂-_{bb};
provided that L contains 1-7 L^{A1}; and wherein bb represents the point of attachment to Ring C.

35.-36. (canceled)

37. The compound of claim 34, wherein L is a divalent group of Formula (L-1): 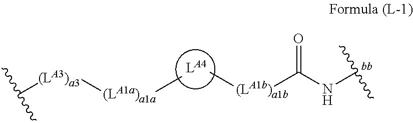
wherein:
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 1 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
bb represents the point of attachment to Ring C.

38.-40. (canceled)

41. The compound of claim 37, wherein a1a+a1b is 3 or 4; or a1a+a1b is 2 or 5.

42.-43. (canceled)

44. The compound of claim 34, wherein L is a divalent group of Formula (L-2): 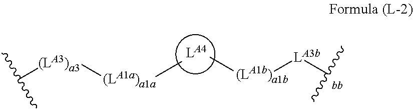
wherein:
a3a is 0 or 1;
L^{A3a} and L^{A3b} are independently selected from the group consisting of: —O—, —N(H)—, and —N(C₁₋₃ alkyl)-;
a1a and a1b are independently integers from 0 to 5, provided that a1a+a1b is from 2 to 7;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
bb represents the point of attachment to Ring C.

45.-50. (canceled)

51. The compound of claim 1, wherein L is:
-(L^{A3})₀₋₁-(L^{A1})₀₋₅-L^{A4}-(L^{A1})₀₋₅-L^{A4}-_{bb}; or
-(L^{A3})₀₋₁-(L^{A1})₀₋₅-L^{A4}-(L^{A1})₀₋₅-L^{A3}-L^{A4}-_{b}b;
provided that L contains 2-7 L^{A1}; and wherein bb represents the point of attachment to Ring C.

52. The compound of claim 51, wherein L is:
-(L^{A3})₀₋₁-(L^{A1})₀₋₅-L^{A4}-(L^{A1})₀₋₅-(piperazinylene)-_{bb},
-(L^{A3})₀₋₁-(L^{A1})₀₋₅-L^{A4}-(L^{A1})₀₋₅-(piperidinylene)-_{bb}; or
-(L^{A3})₀₋₁-(L^{A1})₀₋₅-L^{A4}-(L^{A1})₀₋₅-C(═O)-(piperazinylene)-_{bb};
provided that L contains 2-5 L^{A1};
wherein the piperazinylene and piperidinylene are each optionally substituted with 1-3 R^{a}, and
wherein bb represents the point of attachment to Ring C.

53. The compound of claim 51, wherein L is a divalent group of Formula (L-3), (L-3a), (L-3b), or (L-4): 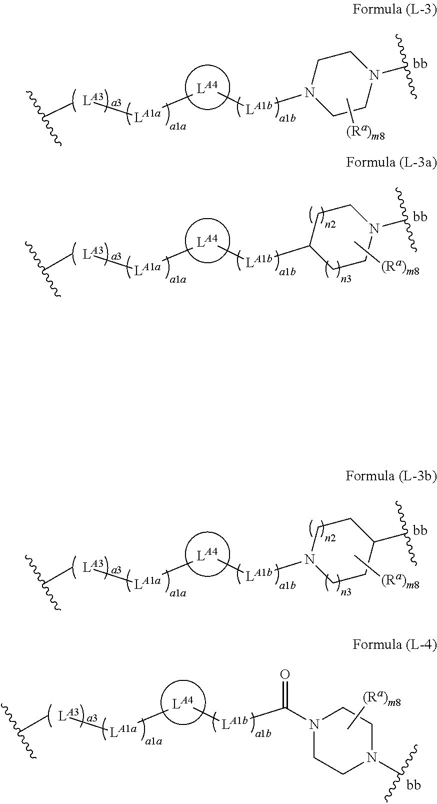
wherein:
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
n2 and n3 are independently 0, 1, or 2;
m8 is 0, 1 or 2;
L^{A4b} is 7-10 membered bicyclic nitrogen-containing heterocyclylene optionally substituted with 1-3 R^{a}; and
bb represents the point of attachment to Ring C.

54.-55. (canceled)

56. The compound of claim 53, wherein a1a+a1bis 3 or 4.

57. The compound of claim 53, wherein a1a+a1b is 2.

58.-68. (canceled)

69. The compound of claim 30, wherein L is:
-(L^{A3})₀₋₁-(L^{A1})₂₋₉-(L^{A3})₀₋₁-(piperazinylene)-_{bb},
wherein bb represents the point of attachment to Ring C.

70. The compound of claim 69, wherein L is a divalent group of Formula (L-6): 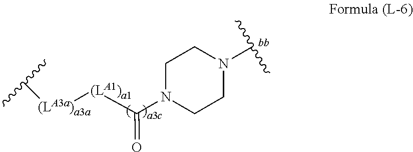
wherein:
L^{A3a} is selected from the group consisting of: —O—, —N(H)—, and —N(C₁₋₃ alkyl)-;
a3a is 0 or 1;
a1 is an integer from 2 to 11;
a3c is 0 or 1; and
bb represents the point of attachment to Ring C.

71.-78. (canceled)

79. The compound of claim 1, wherein the compound of Formula (I) is a compound of Formula (I-A): 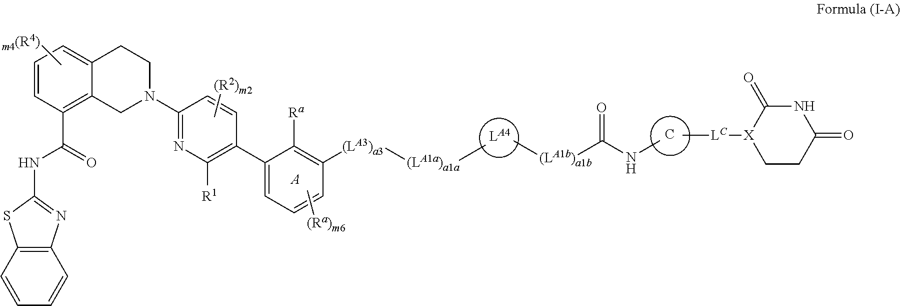
or a pharmaceutically acceptable salt thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 1 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; or
the compound of Formula (I) is a compound of Formula (I-B): 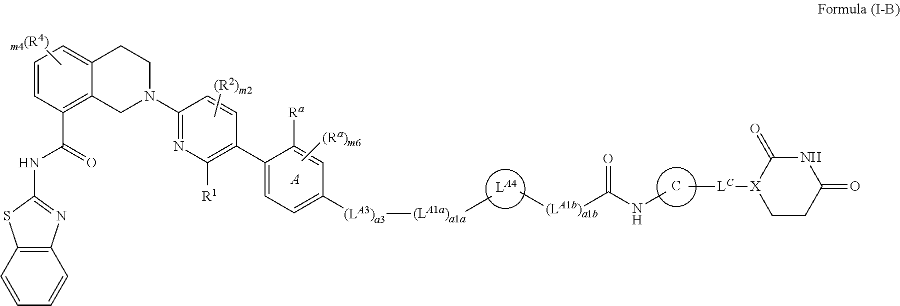
or a pharmaceutically acceptable salt thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 1 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; or
the compound of Formula (I) is a compound of Formula (I-C) or (I-D): 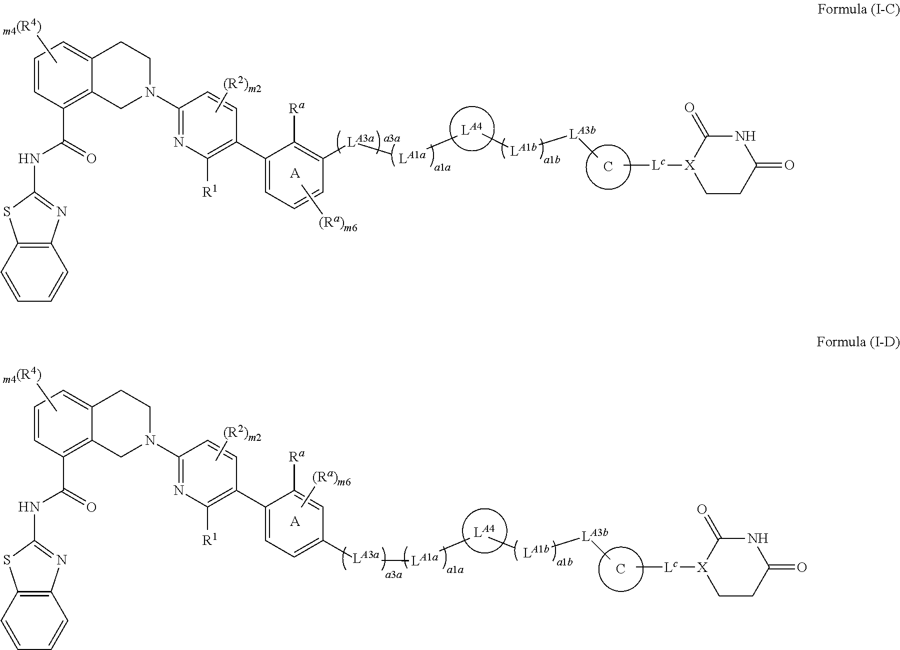
or a pharmaceutically acceptable salt thereof, wherein:
m6 is 0 or 1;
a3a is 0 or 1;
L^{A3a} and L^{A3b} are independently selected from the group consisting of: —O—, —N(H)—, and —N(C₁₋₃ alkyl)-;
a1a and a1b are independently integers from 0 to 5, provided that a1a+a1b is from 2 to 7;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; or
the compound of Formula (is a compound of Formula (I-E): 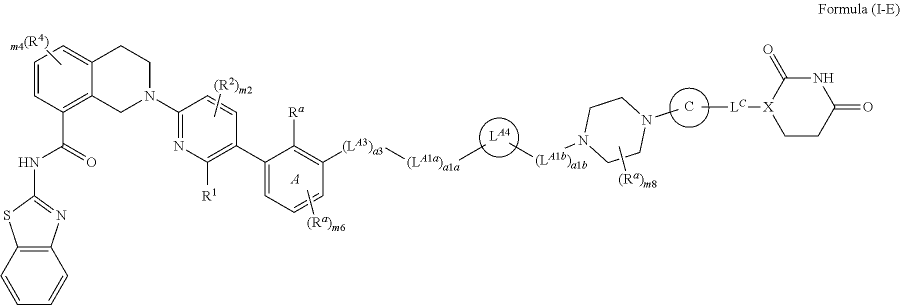
or a pharmaceutically acceptable salt thereof wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
m8 is 0, 1, or 2; or
the compound of Formula is a compound of Formula (I-Ea): 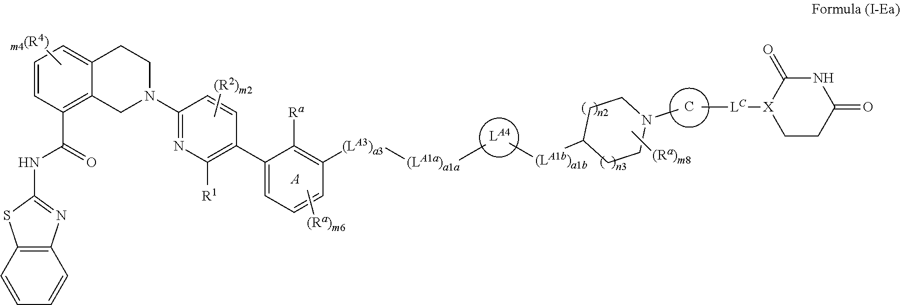
or a pharmaceutically acceptable salt thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, an C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
n2 and n3 are independently 0, 1, or 2; and
m8 is 0, 1, or 2; or
the compound of Formula is a compound of Formula (I-Eb): 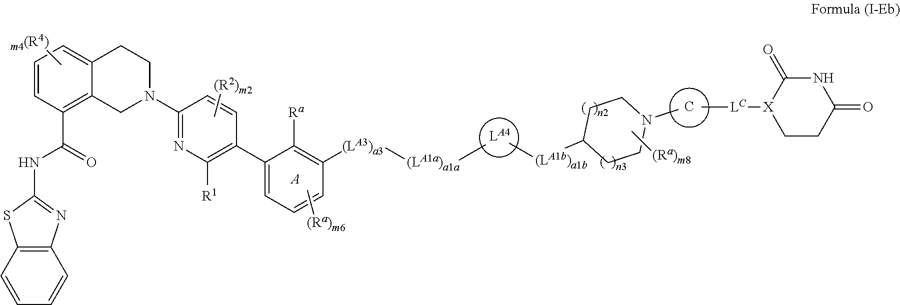
or a pharmaceutically acceptable salt thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
n2 and n3 are independently 0, 1, or 2; and
m8 is 0, 1, or 2; or
the compound of Formula (l) is a compound of Formula (I-F): 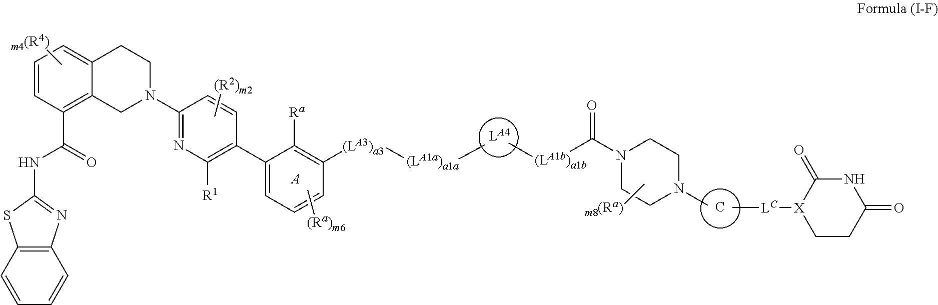
or a pharmaceutically acceptable salt thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
m8 is 0, 1, or 2; or
the compound of Formula (l) is a compound of Formula (I-G): 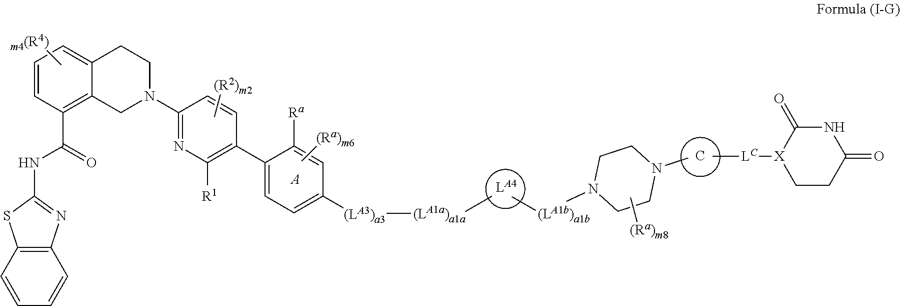
or a pharmaceutically acceptable salt thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
m8 is 0, 1, or 2; or
the compound of Formula (l) is a compound of Formula (I-H): 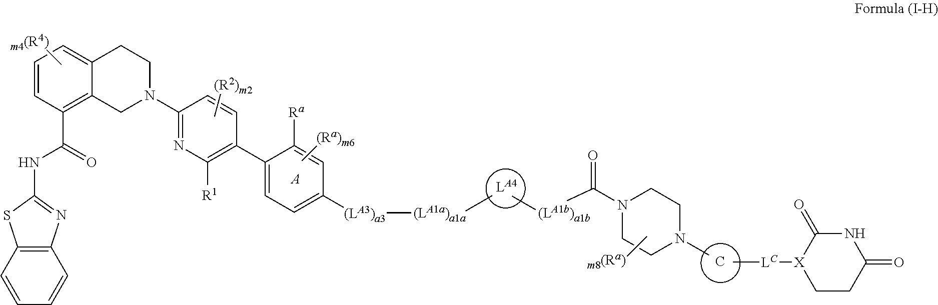
or a pharmaceutically acceptable salt thereof, wherein:
m6 is 0 or 1;
a3 is 0 or 1;
L^{A3} is selected from the group consisting of: —O—, —N(H)—, and —N(C₁₋₃ alkyl)-;
a1a and a1b are independently integers from 0 to 5, provided that a1a+a1b is from 2 to 5;
L^{A1a} and L^{A1b} are independently selected from the group consisting of: —CH₂—, —CHR^{L}—, and —C(R^{L})₂—; and
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a}; and
m8 is 0, 1, or 2.

80. The compound of claim 1, wherein the compound is selected from the group consisting of the following: 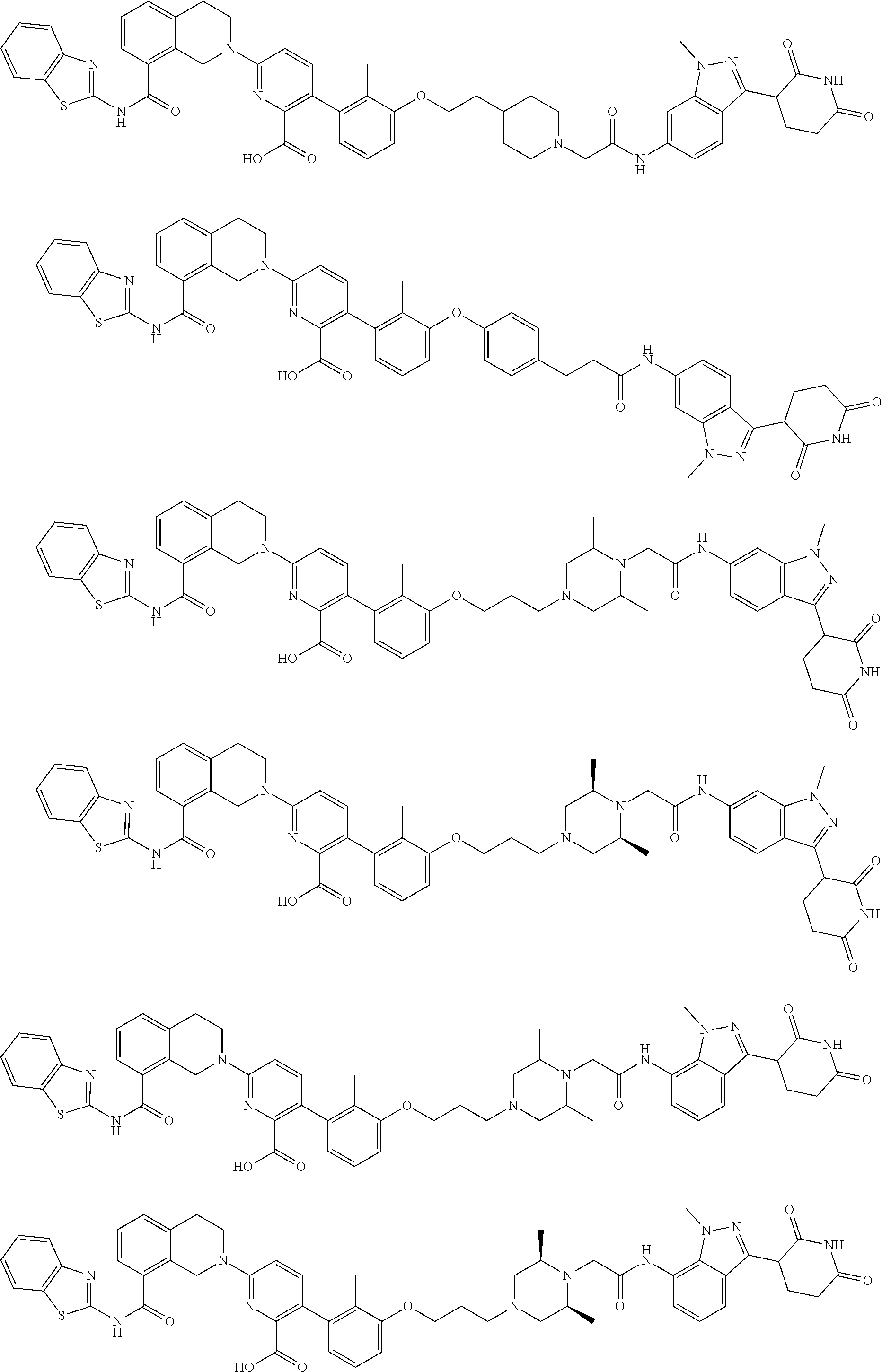 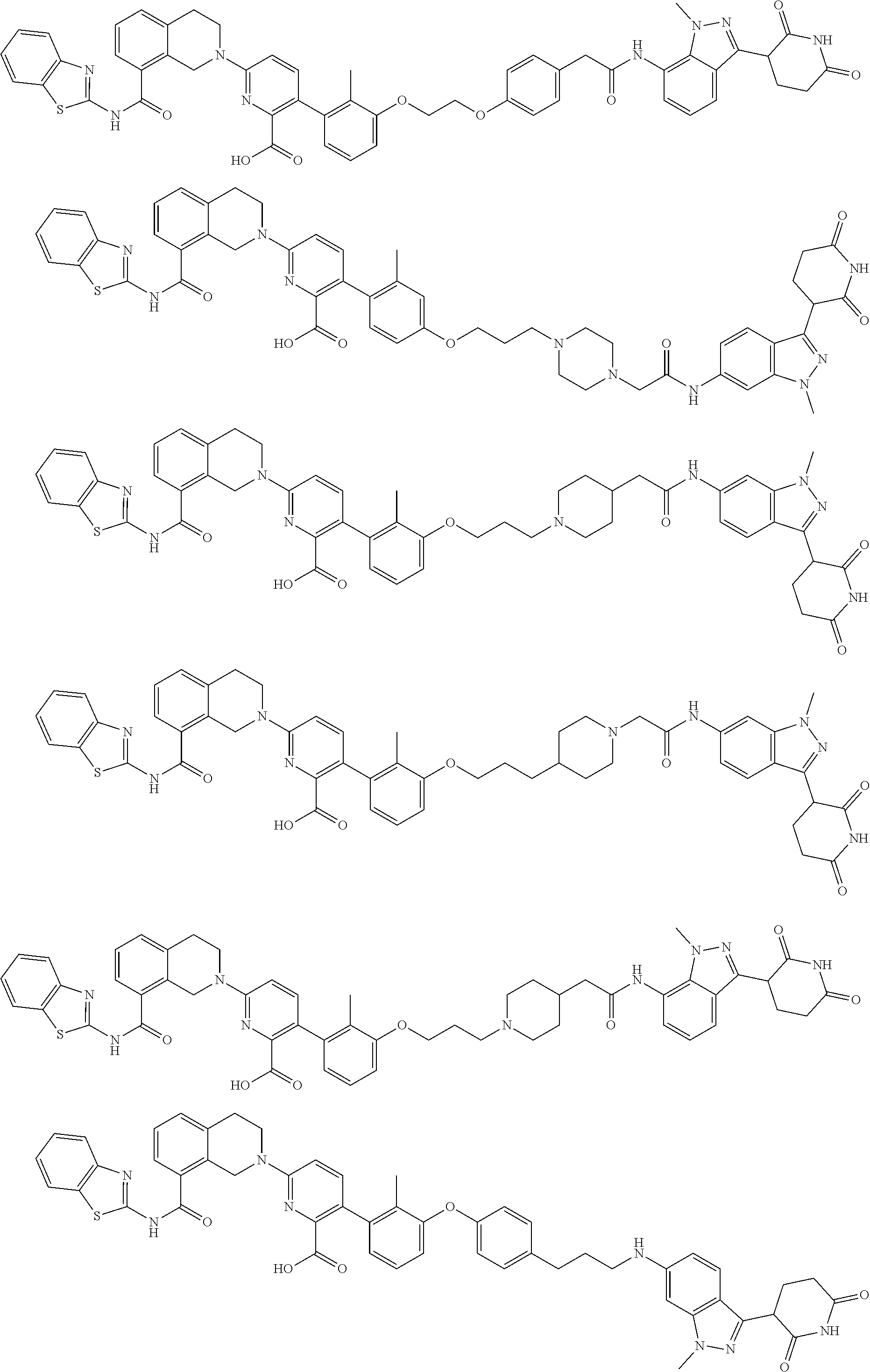 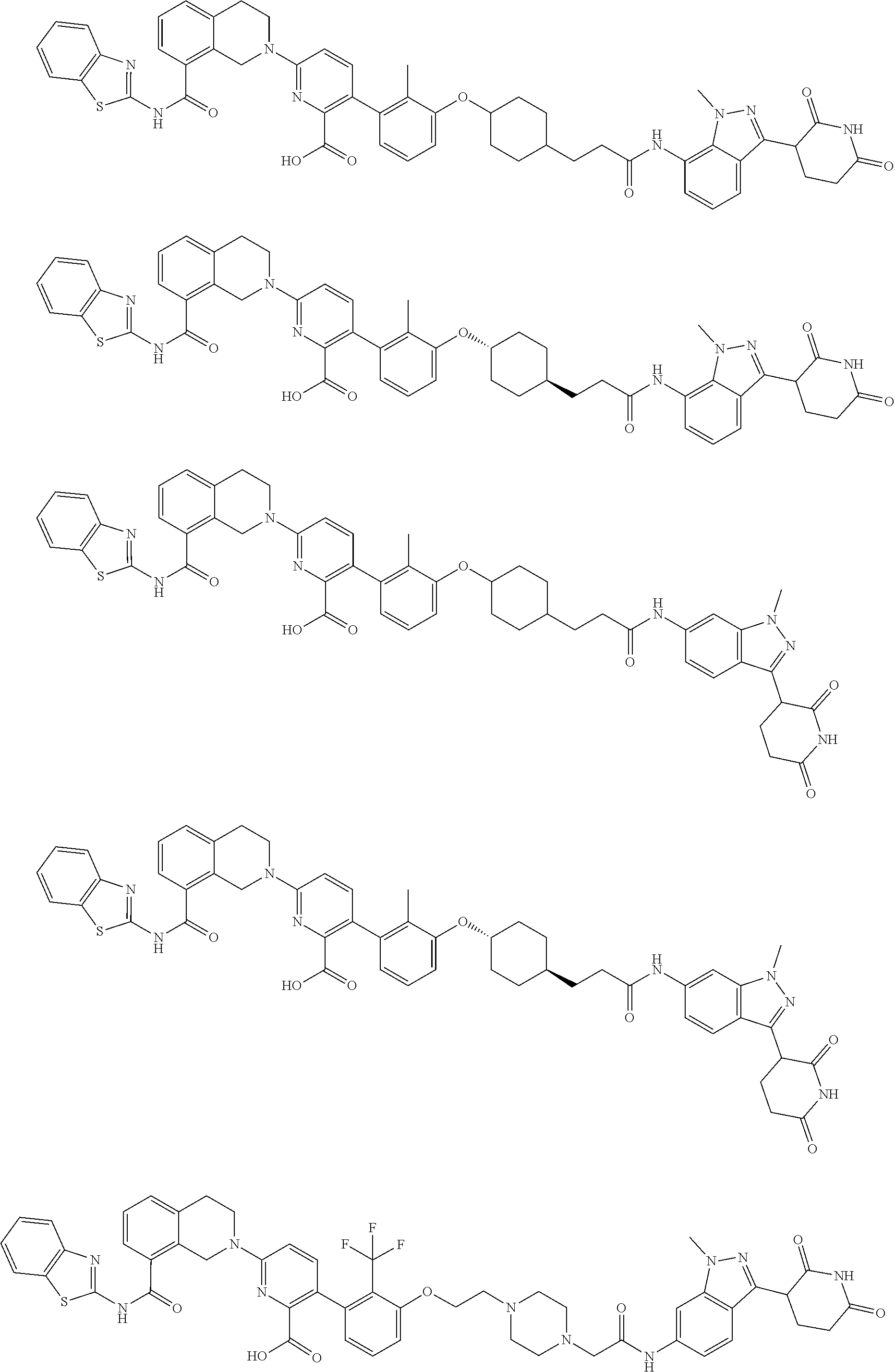 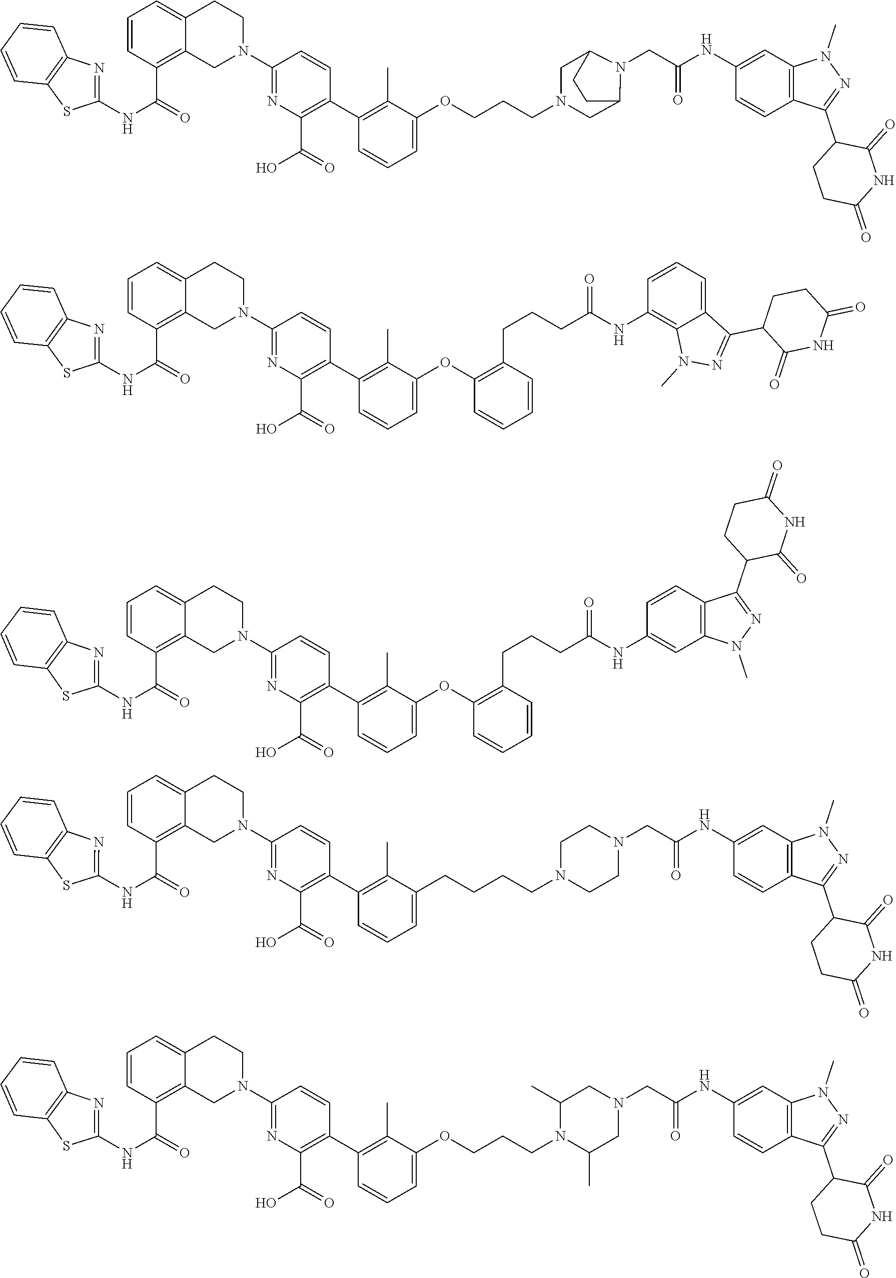 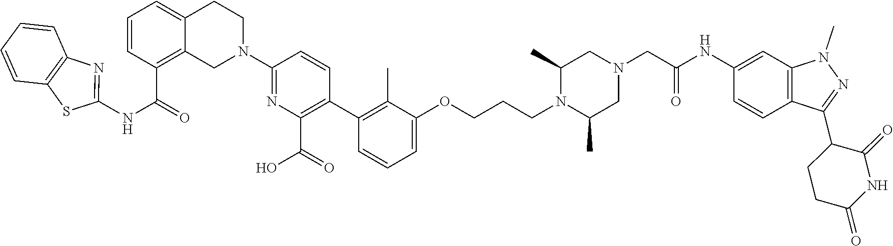 [SMILES: Cc1c(OCCCN2[C@H](C)CN(CC(=O)Nc3ccc4c(C5CCC(=O)NC5=O)nn(C)c4c3)C[C@@H]2C)cccc1-c1ccc(N2CCc3cccc(C(=O)Nc4nc5ccccc5s4)c3C2)nc1C(=O)O] 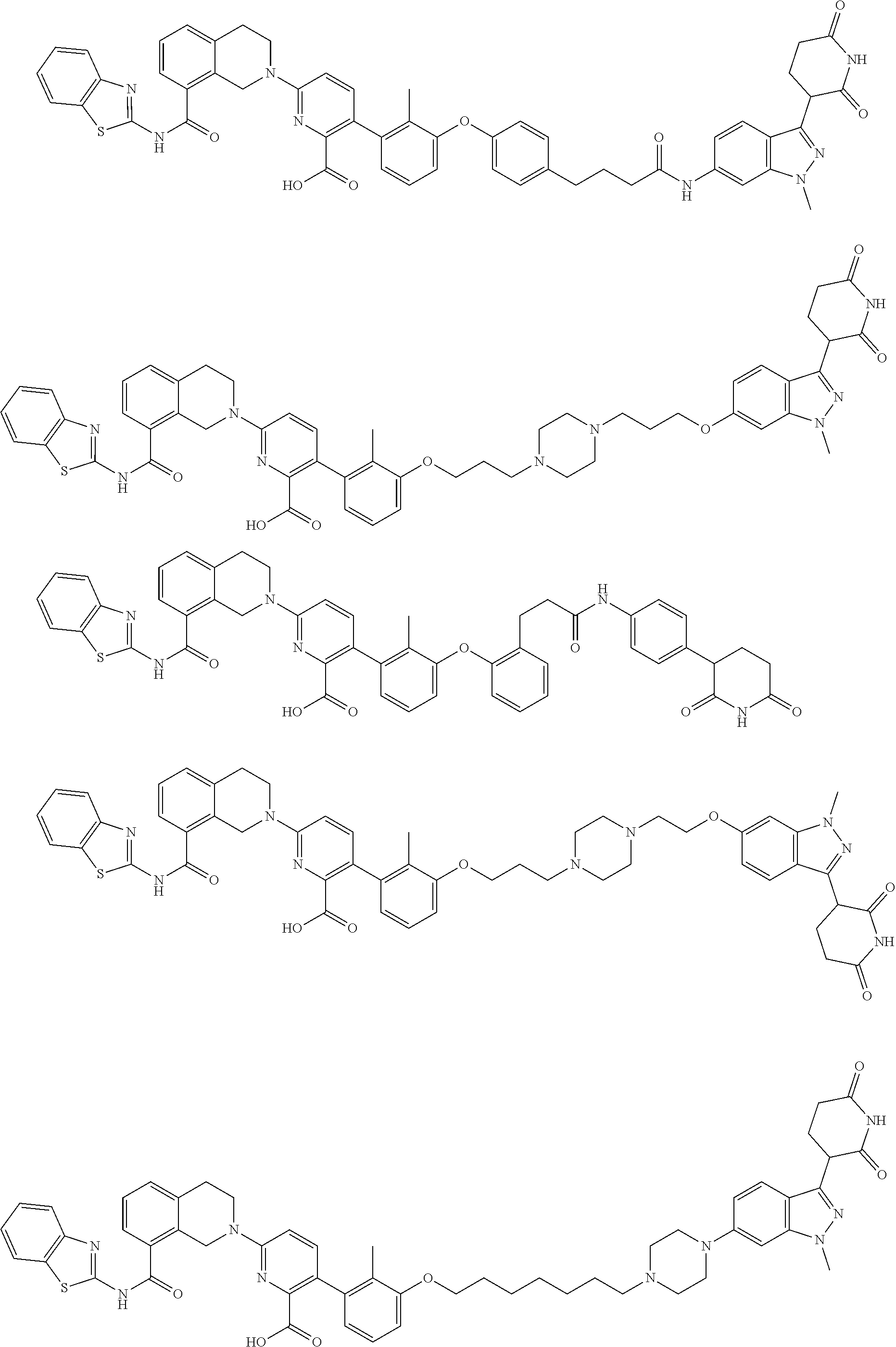 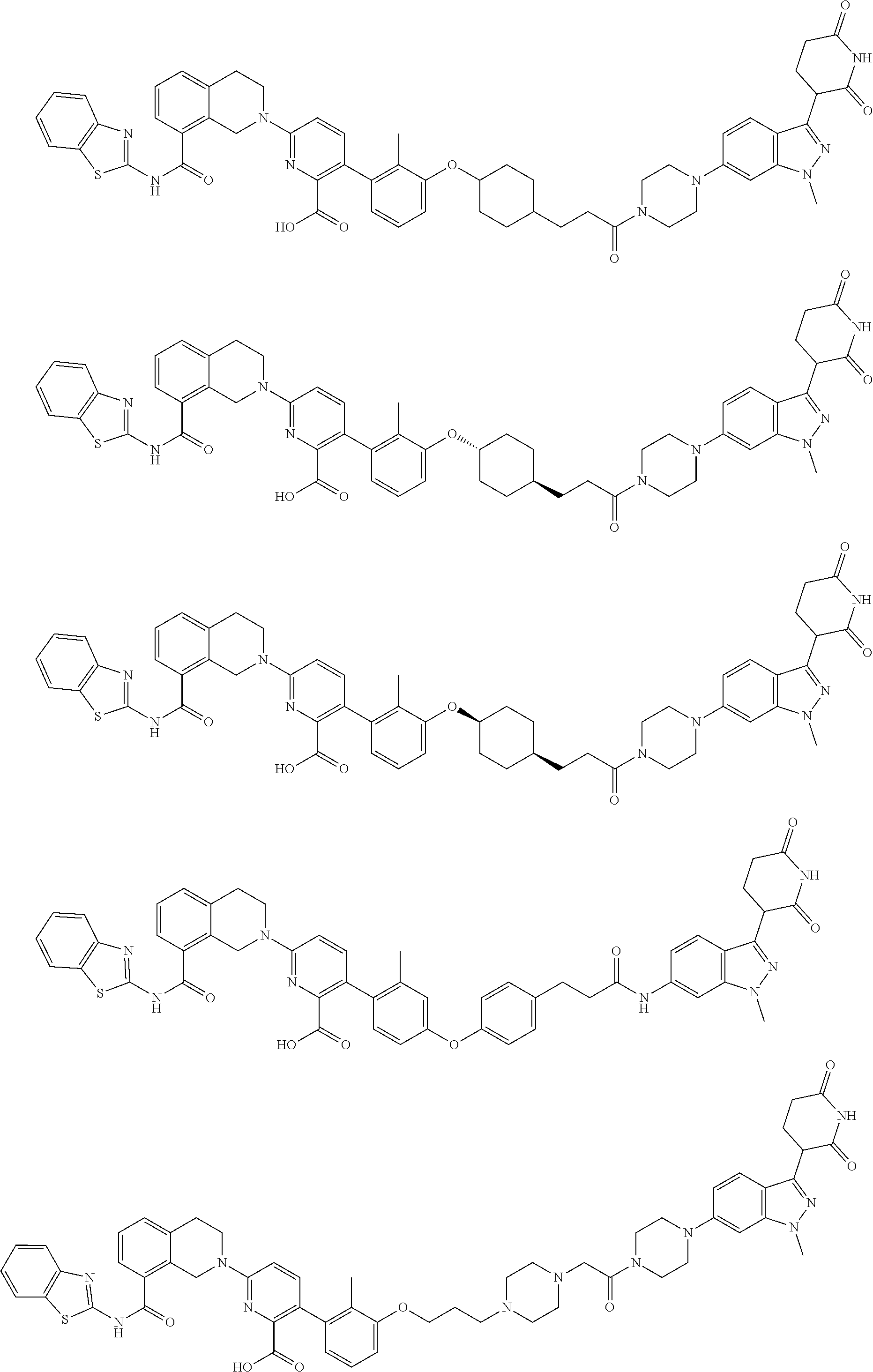 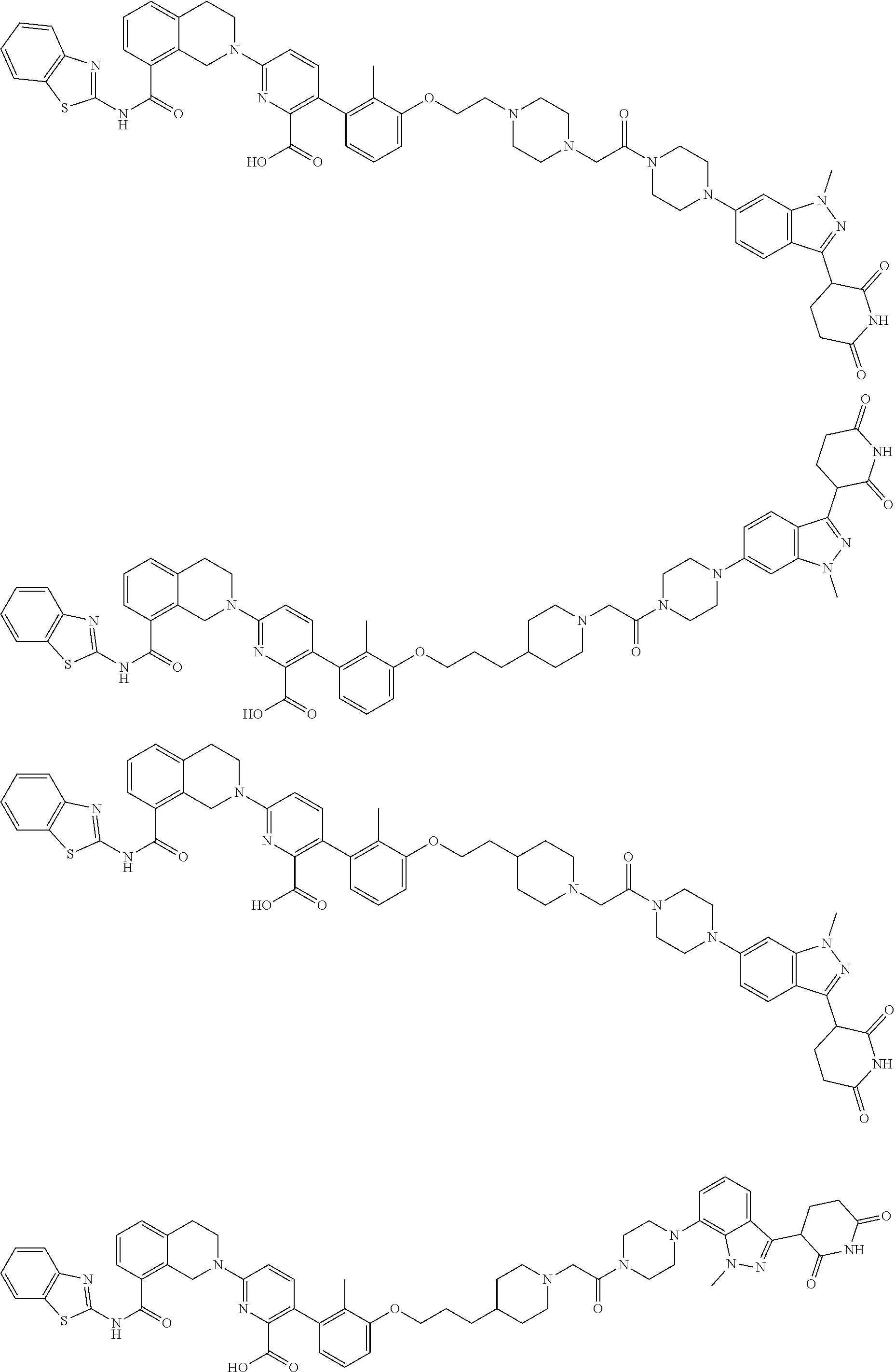 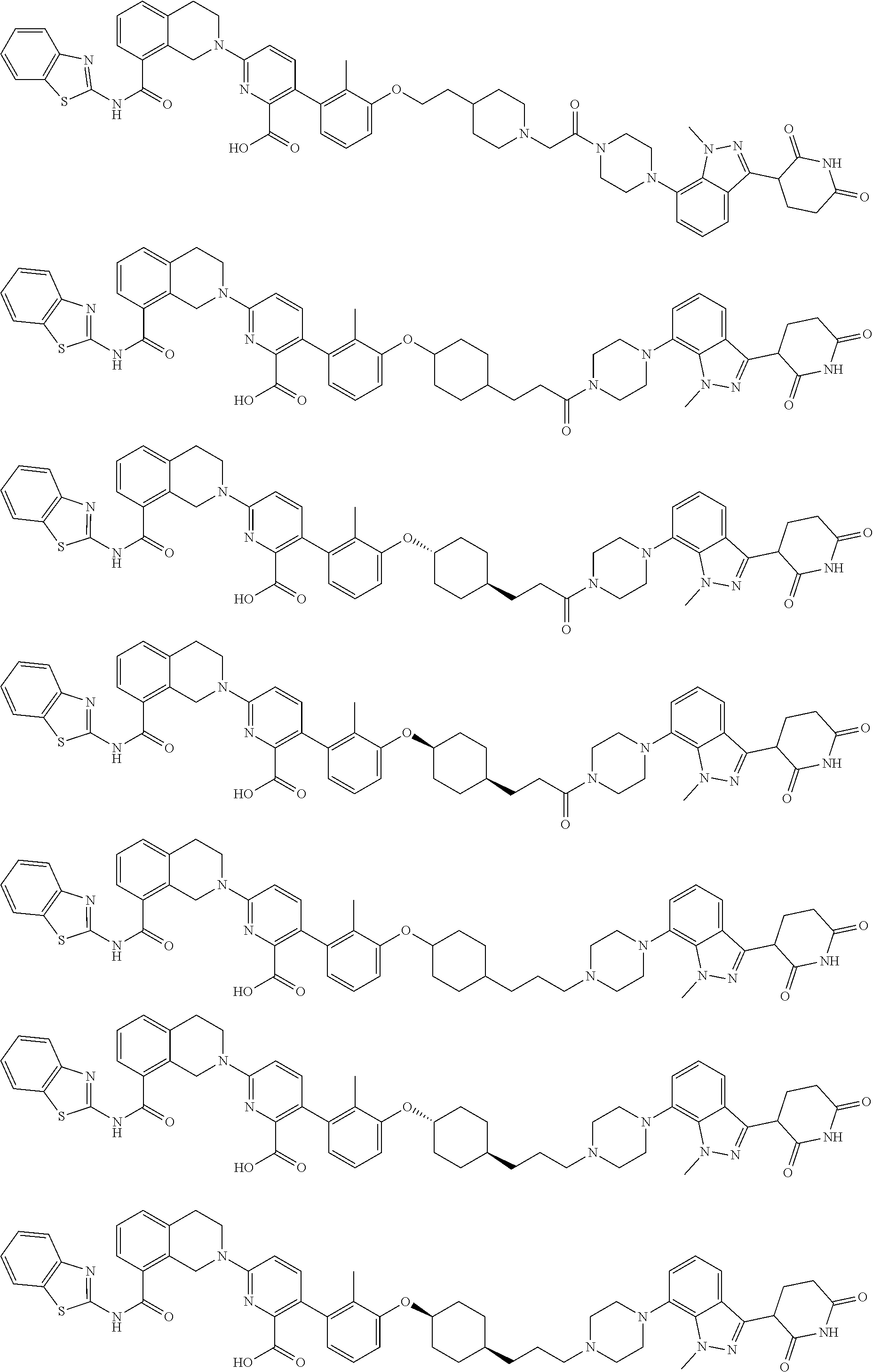 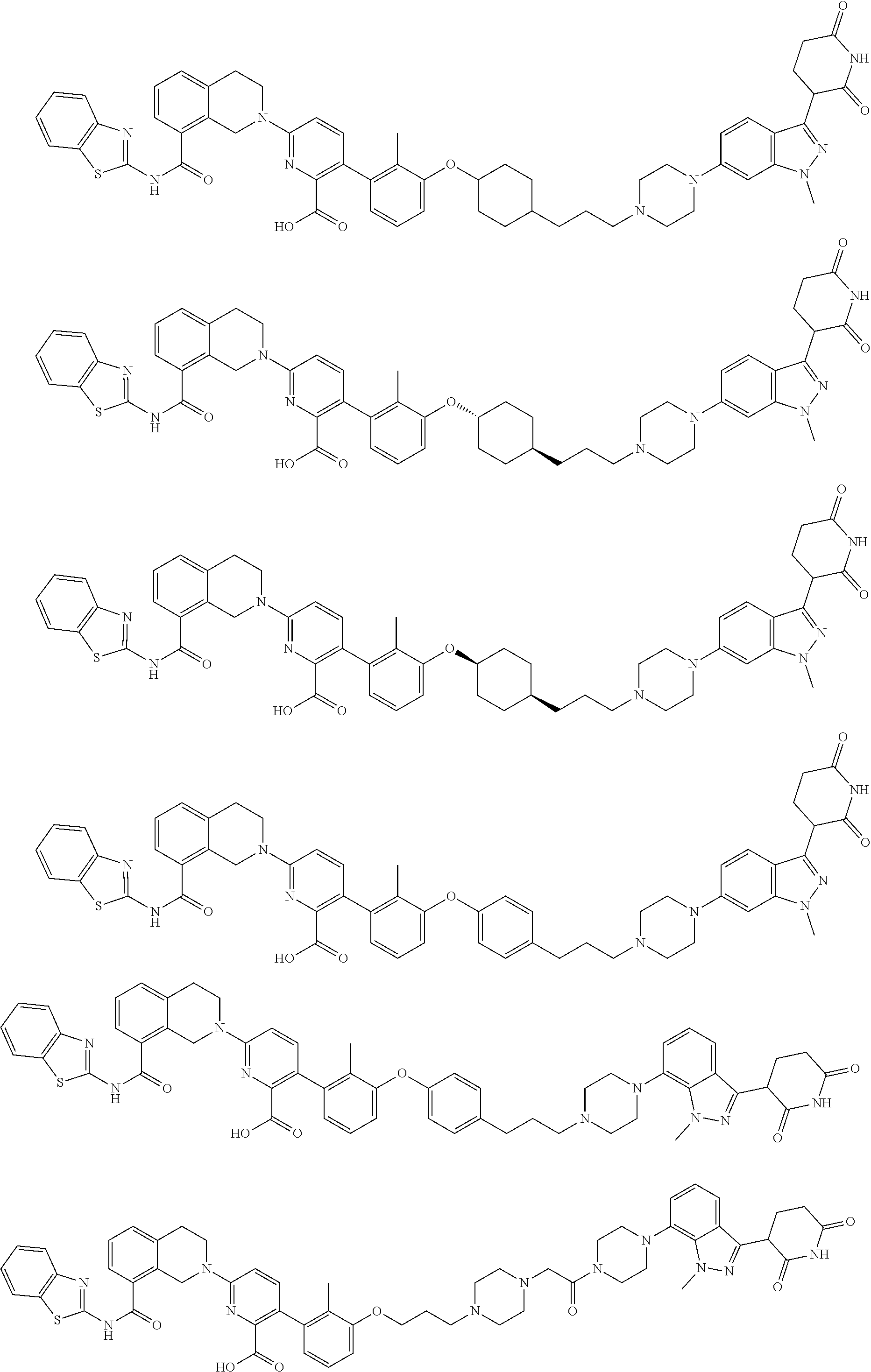 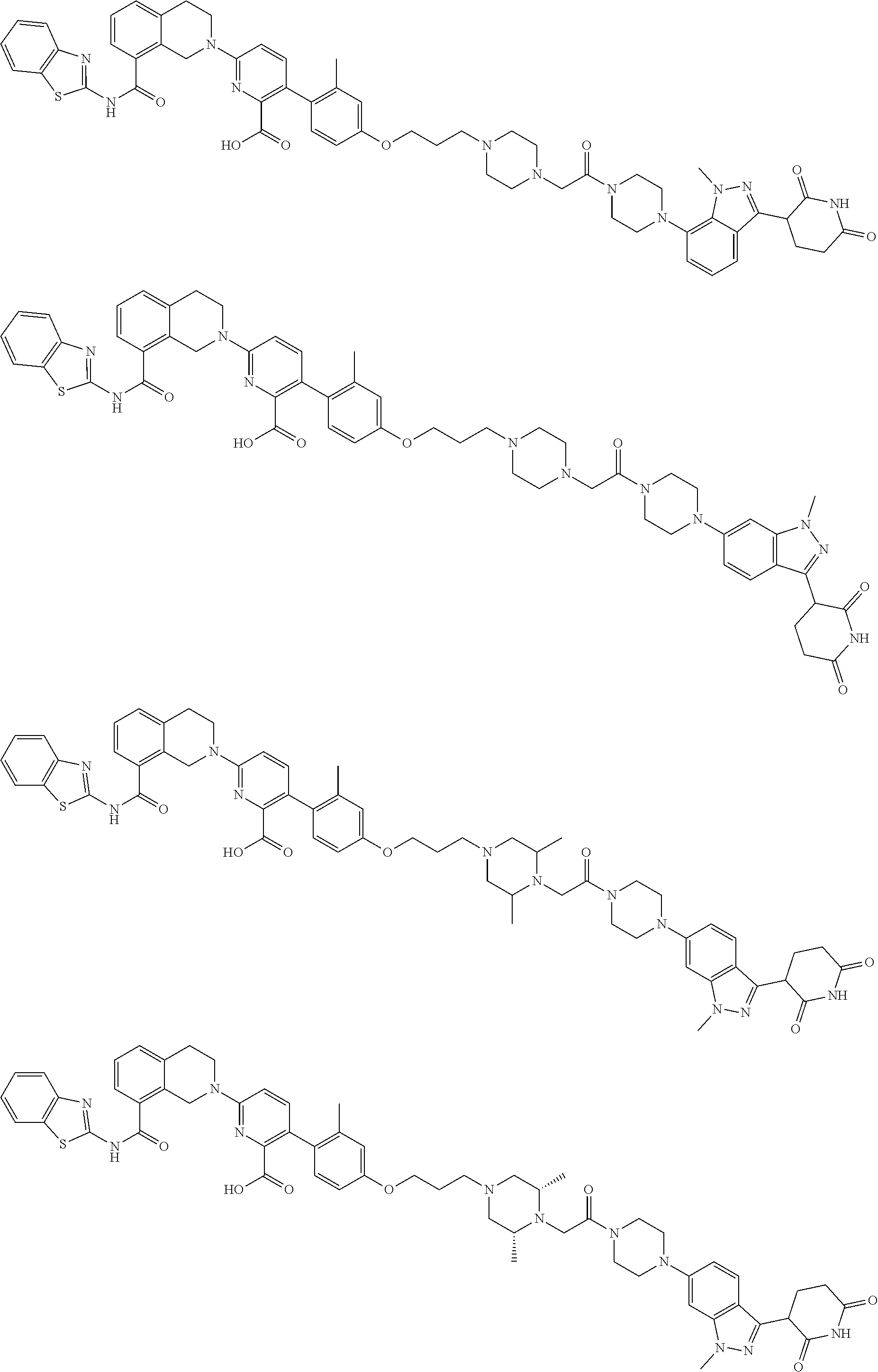 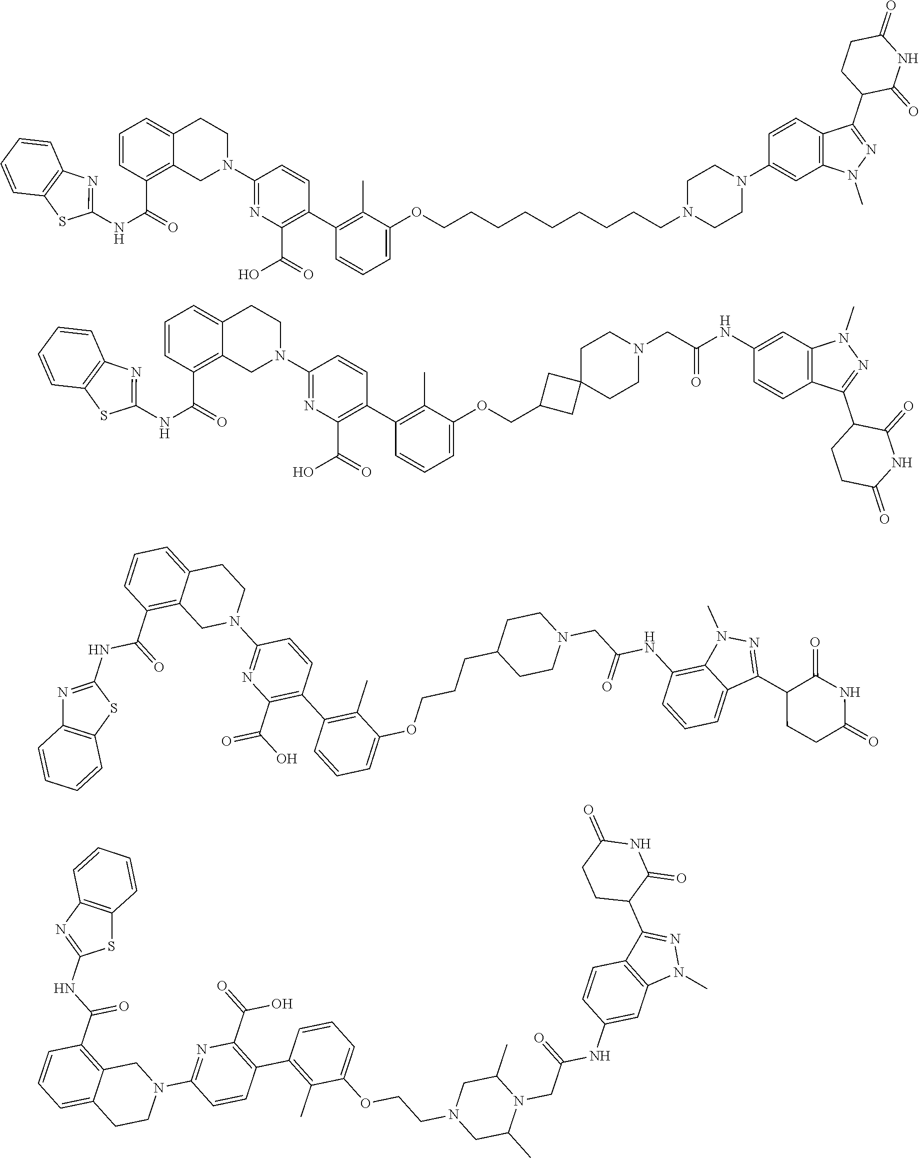 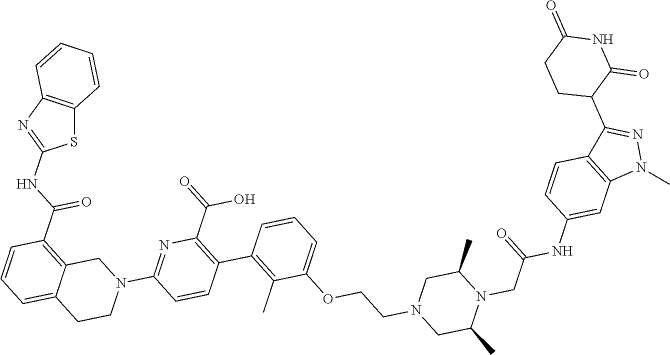 [SMILES: Cc1c(OCCN2C[C@@H](C)N(CC(=O)Nc3ccc4c(C5CCC(=O)NC5=O)nn(C)c4c3)[C@@H](C)C2)cccc1-c1ccc(N2CCc3cccc(C(=O)Nc4nc5ccccc5s4)c3C2)nc1C(=O)O] 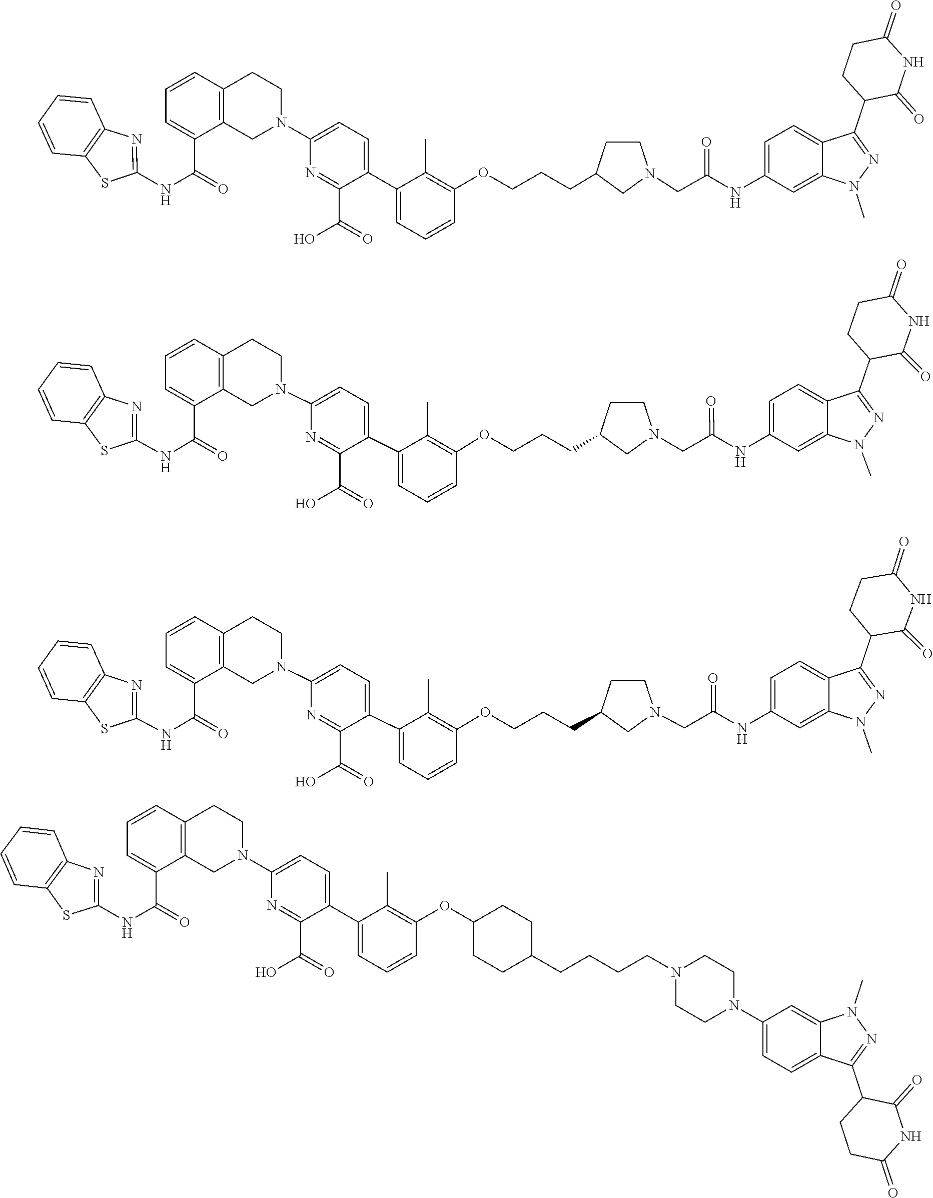 [SMILES: Cc1c(OCCCC2CCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O ; Cc1c(OCCC[C@@H]2CCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O ; Cc1c(OCCC[C@H]2CCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O ; Cc1c(OC2CCC(CCCCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] 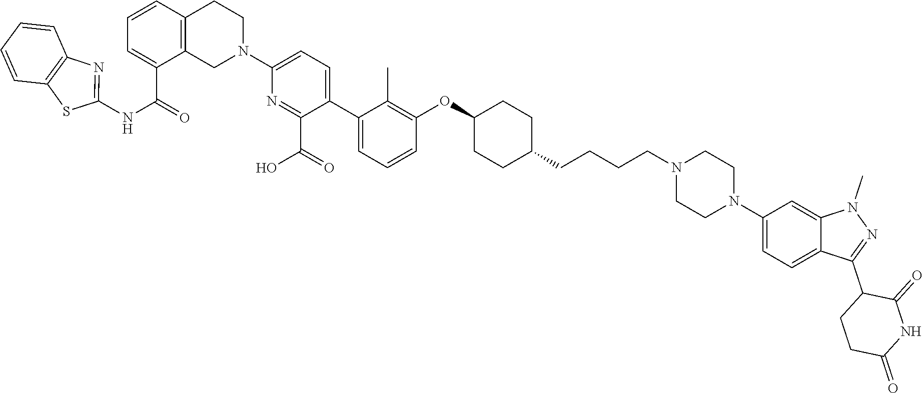 [SMILES: Cc1c(O[C@H]2CC[C@H](CCCCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] 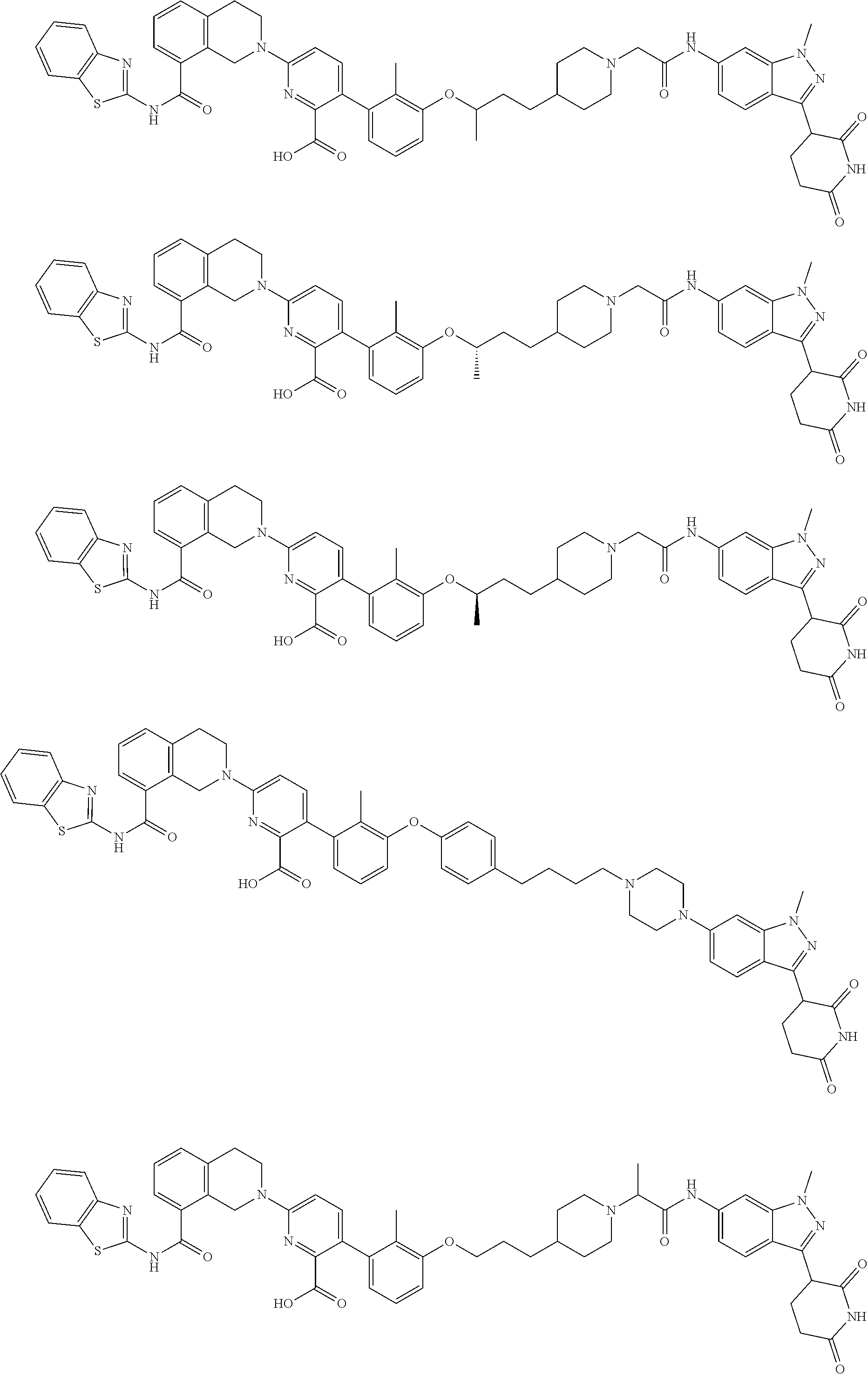 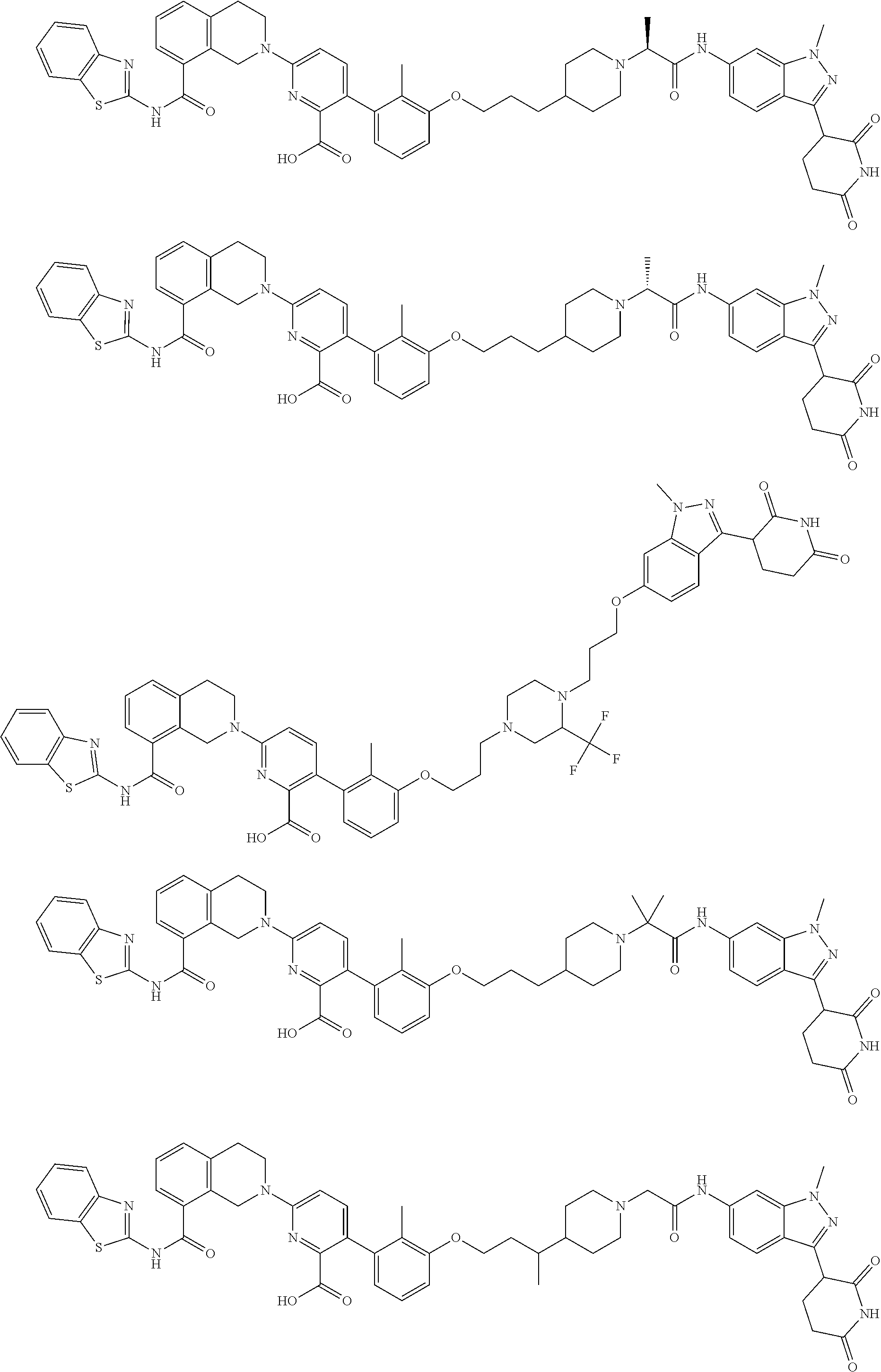 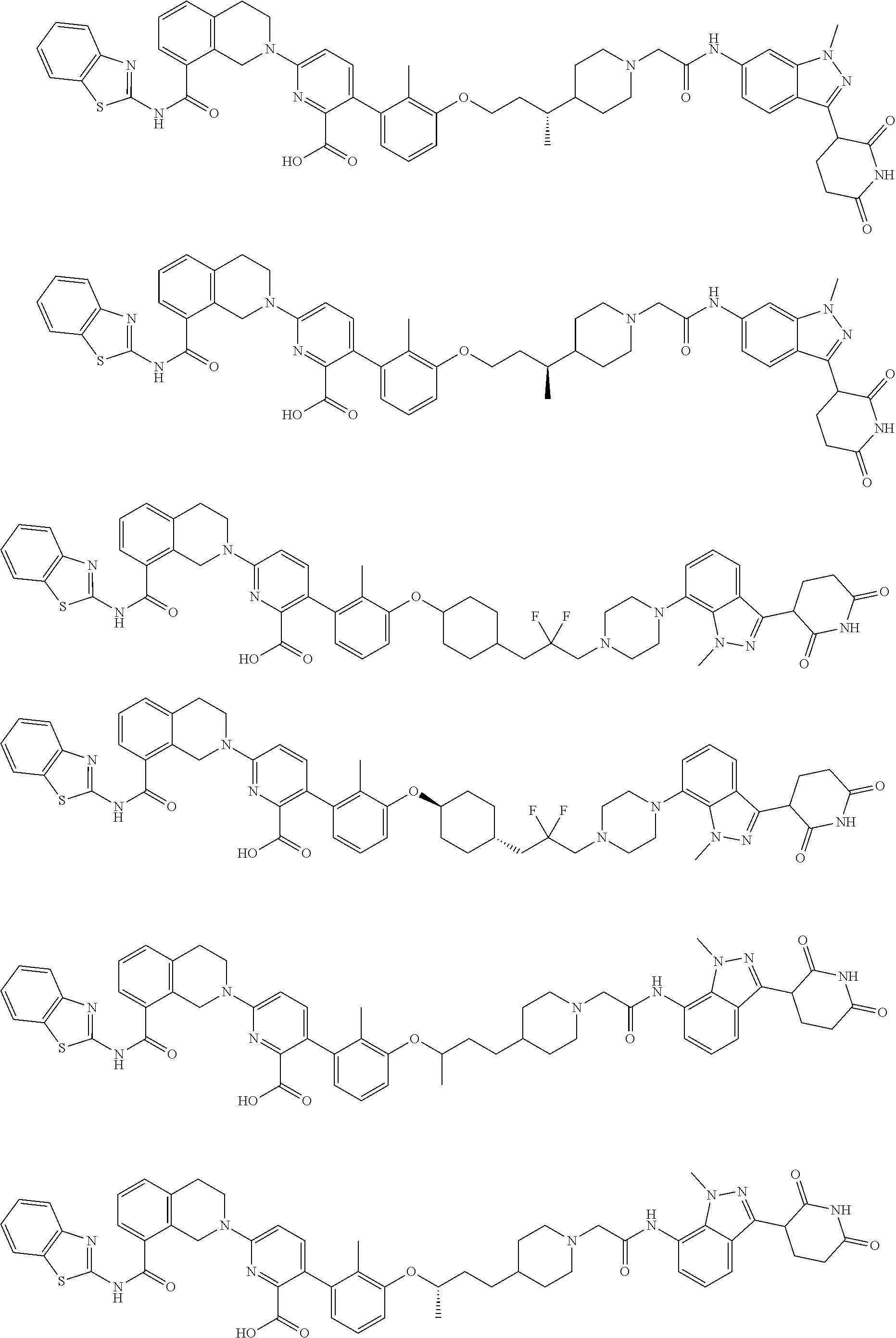 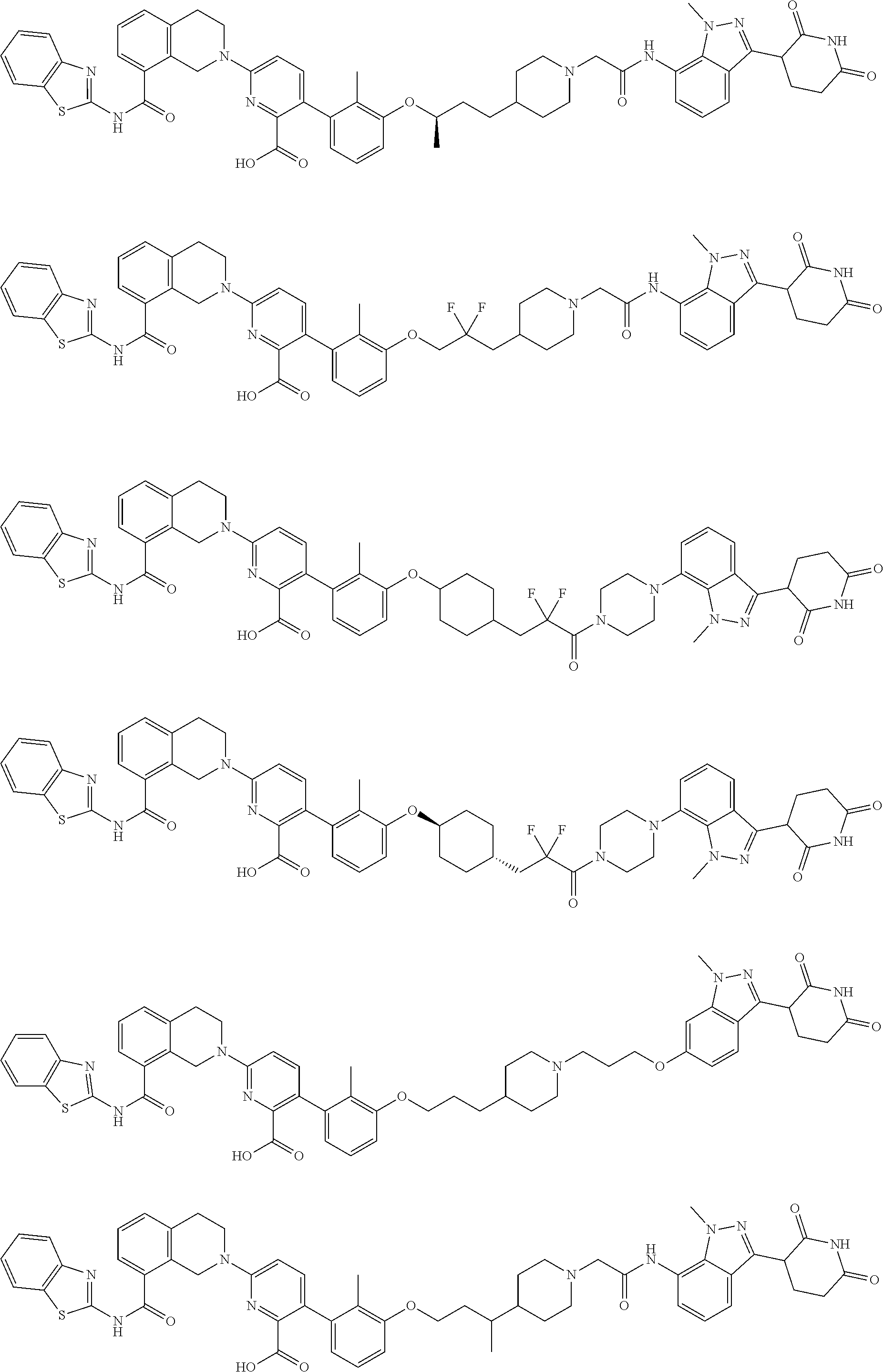 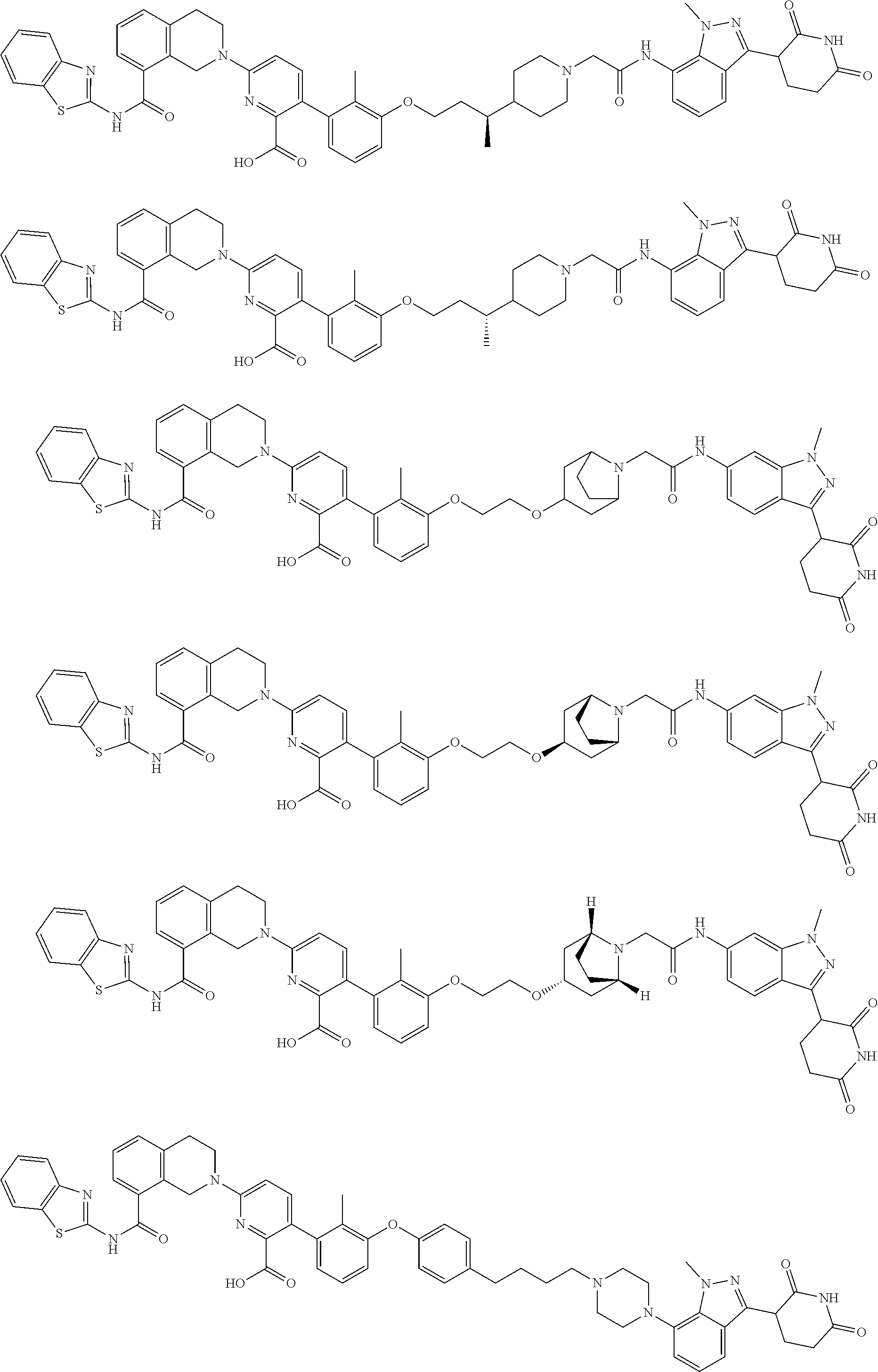 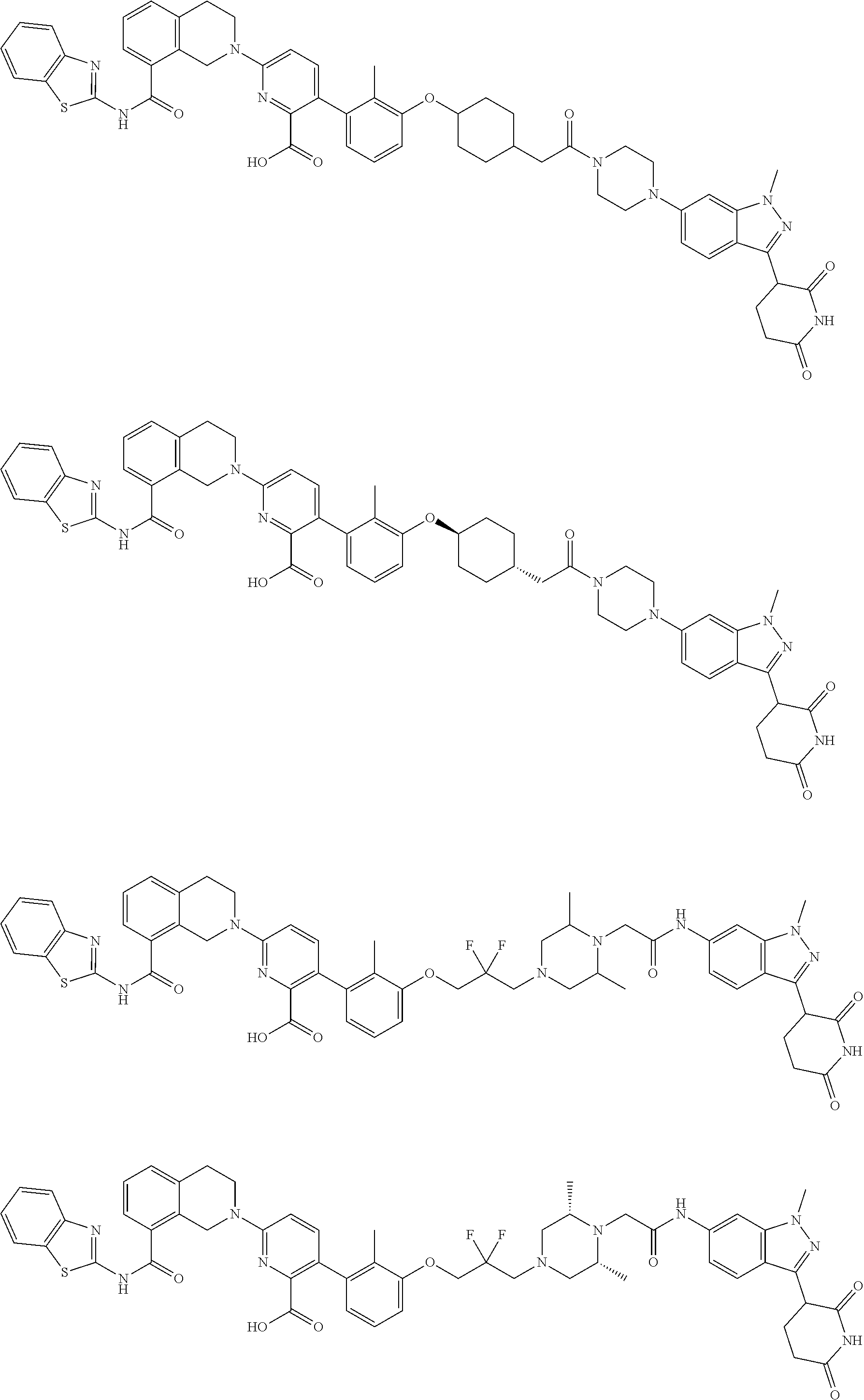 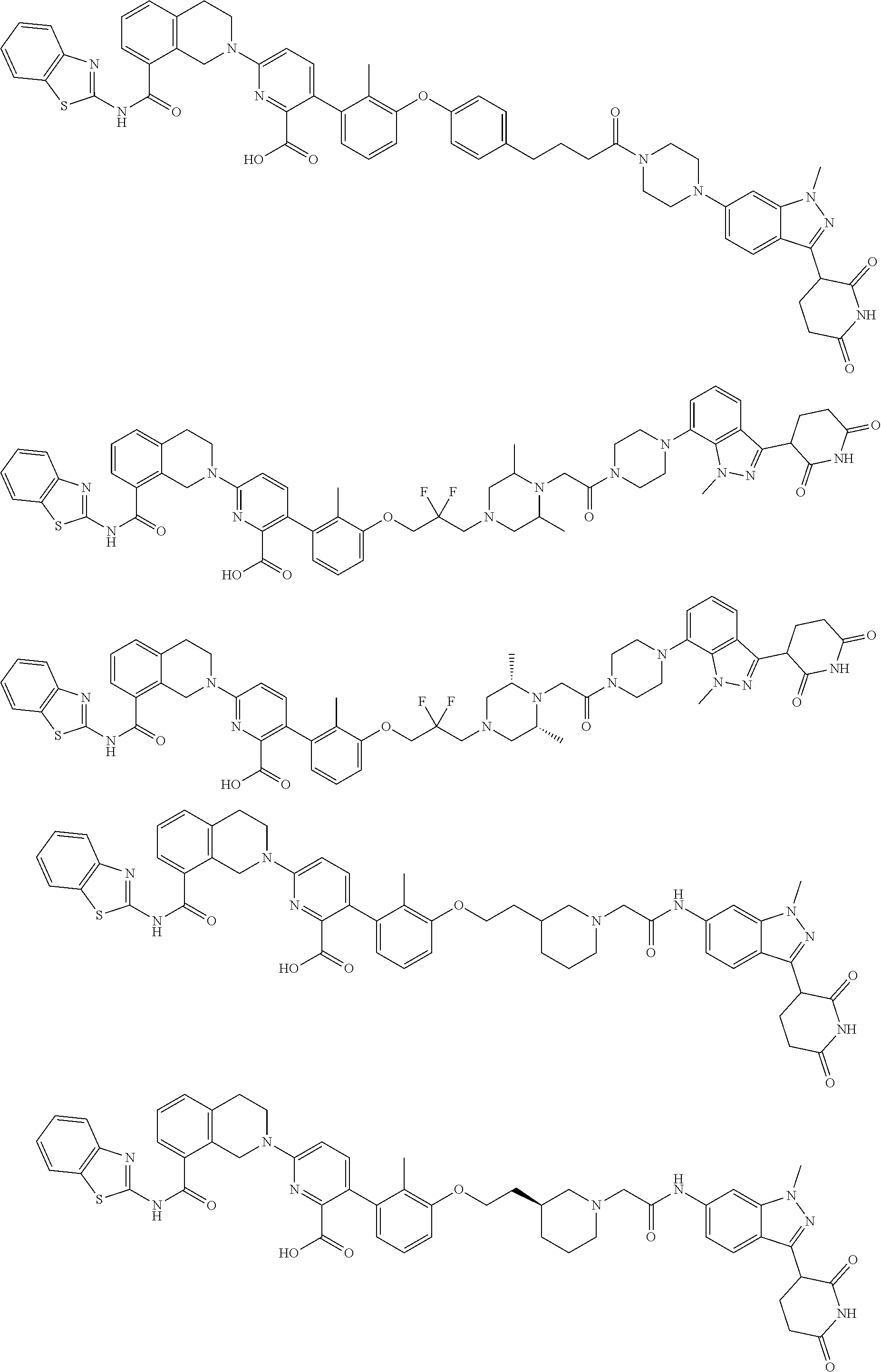 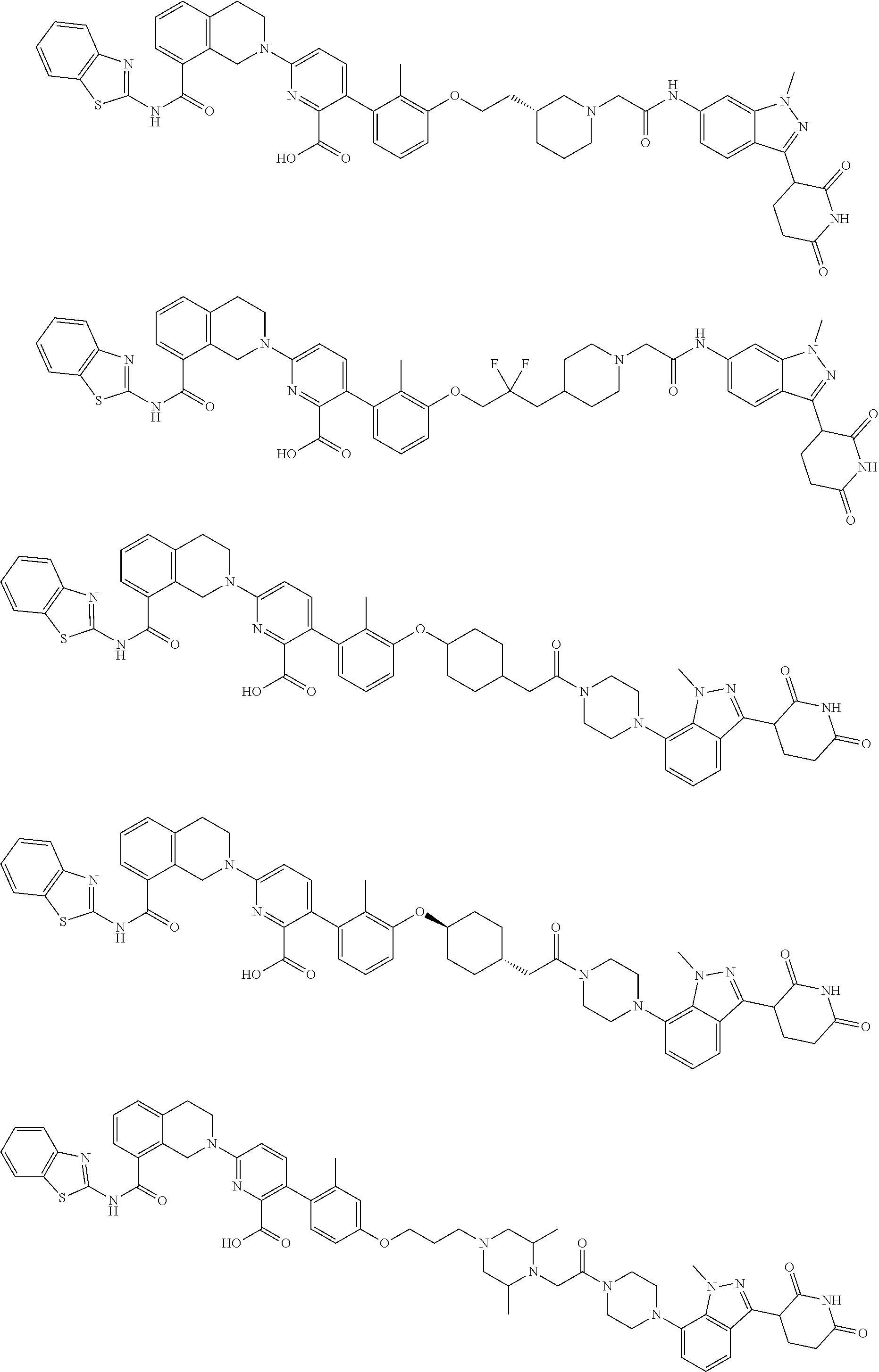 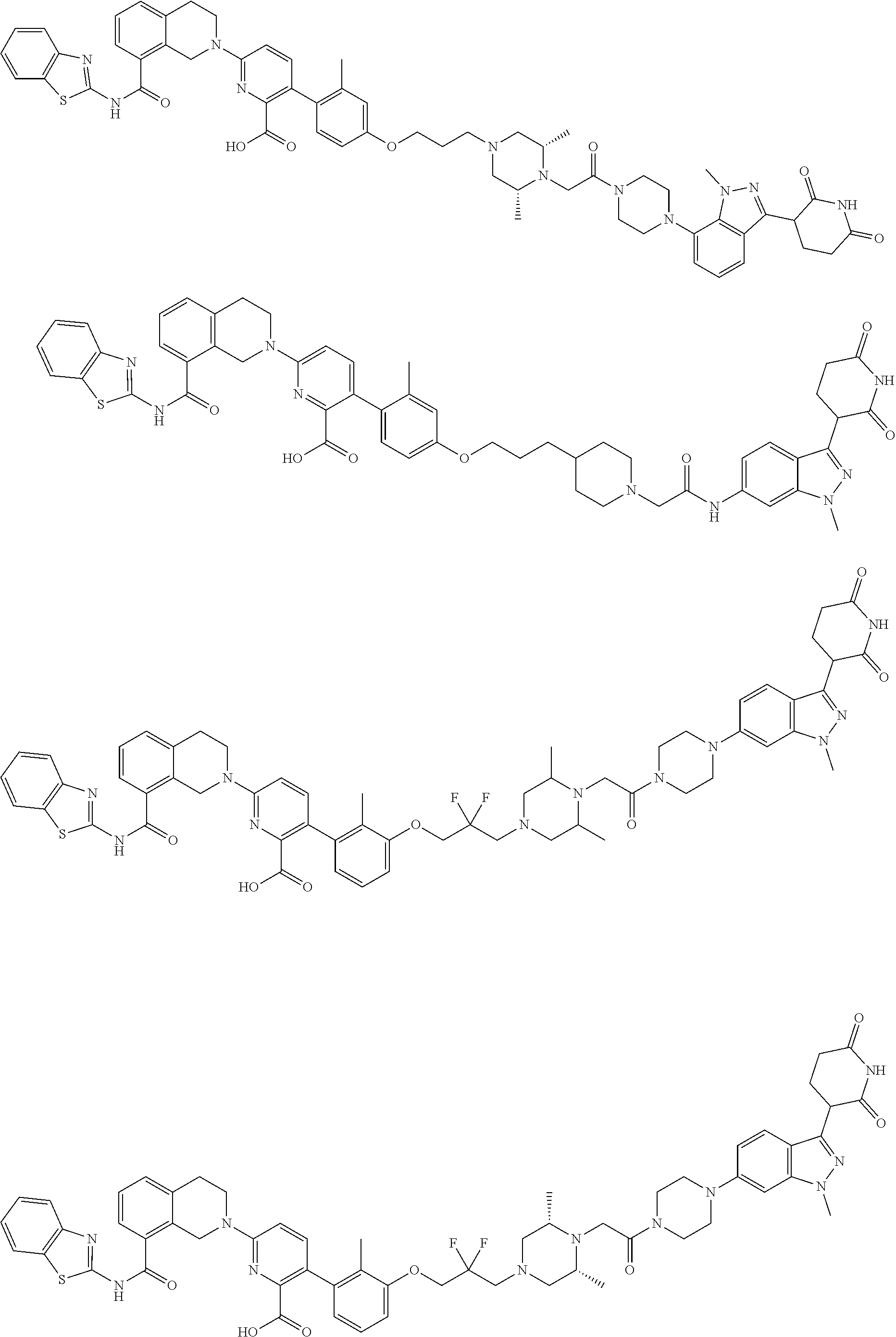 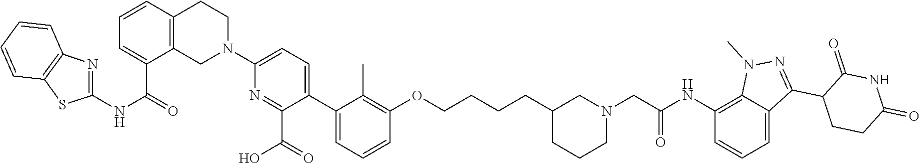 [SMILES: Cc1c(OCCCCC2CCCN(CC(=O)Nc3cccc4c(C5CCC(=O)NC5=O)nn(C)c34)C2)cccc1-c1ccc(N2CCc3cccc(C(=O)Nc4nc5ccccc5s4)c3C2)nc1C(=O)O] 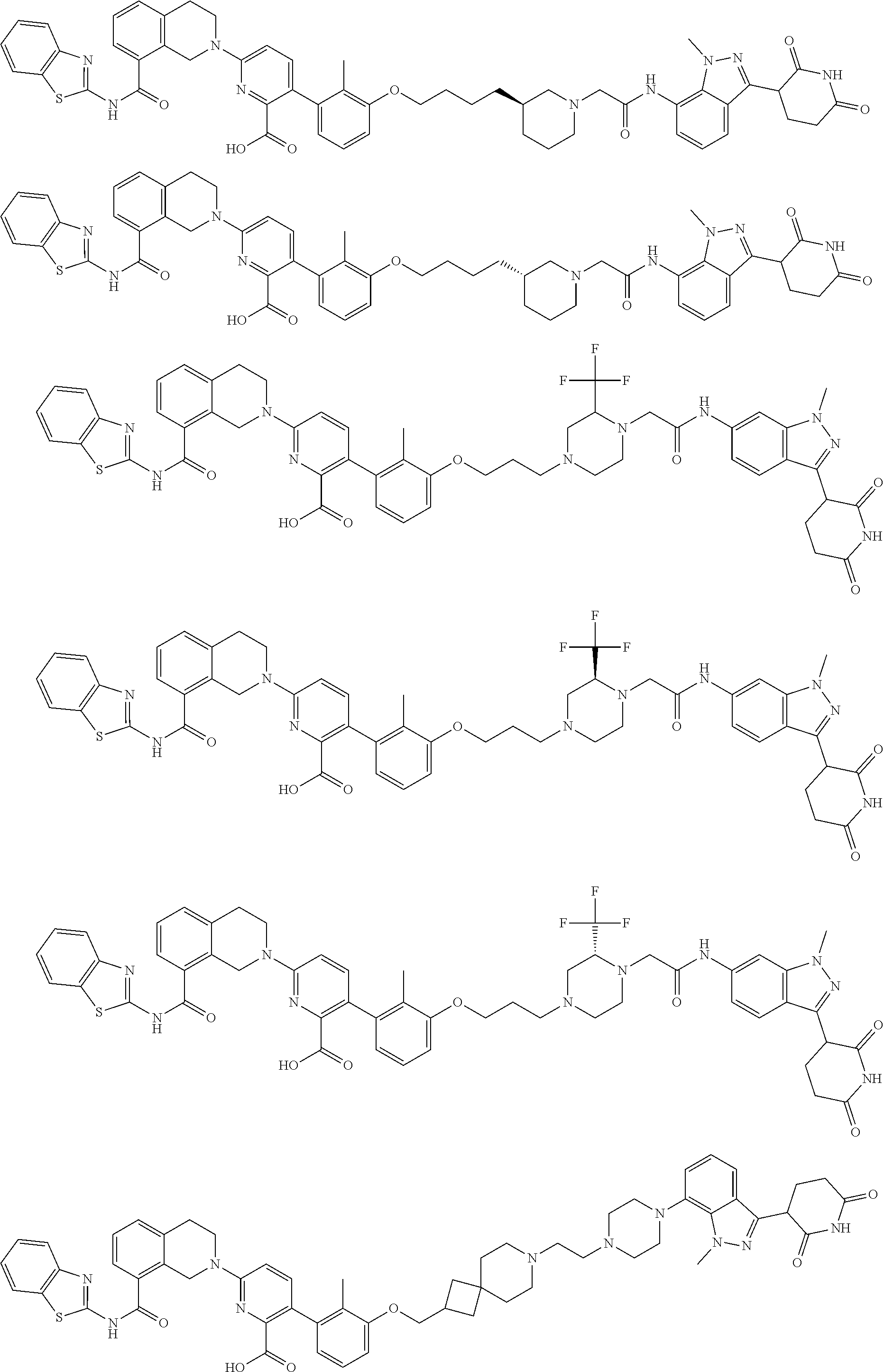 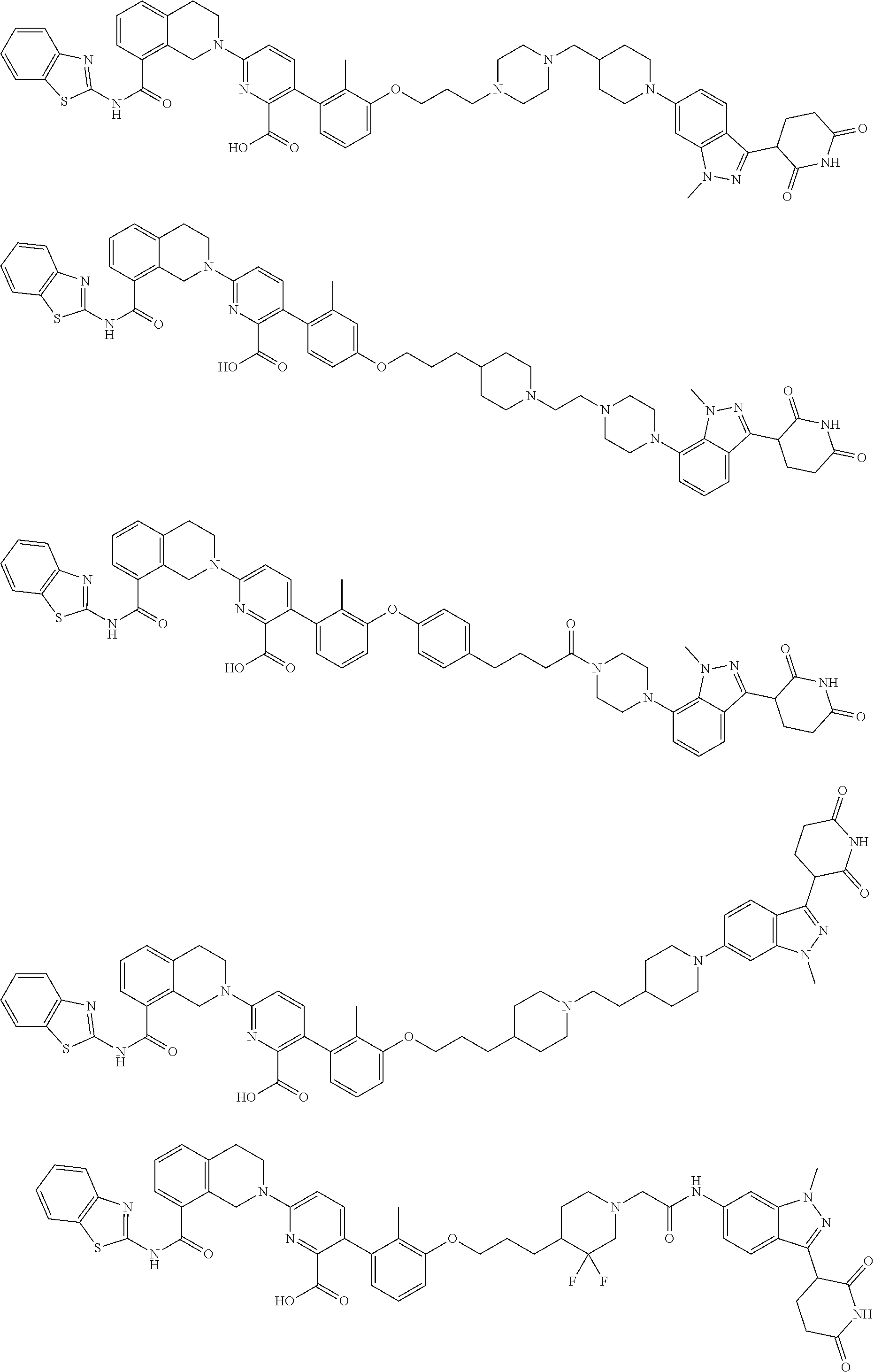 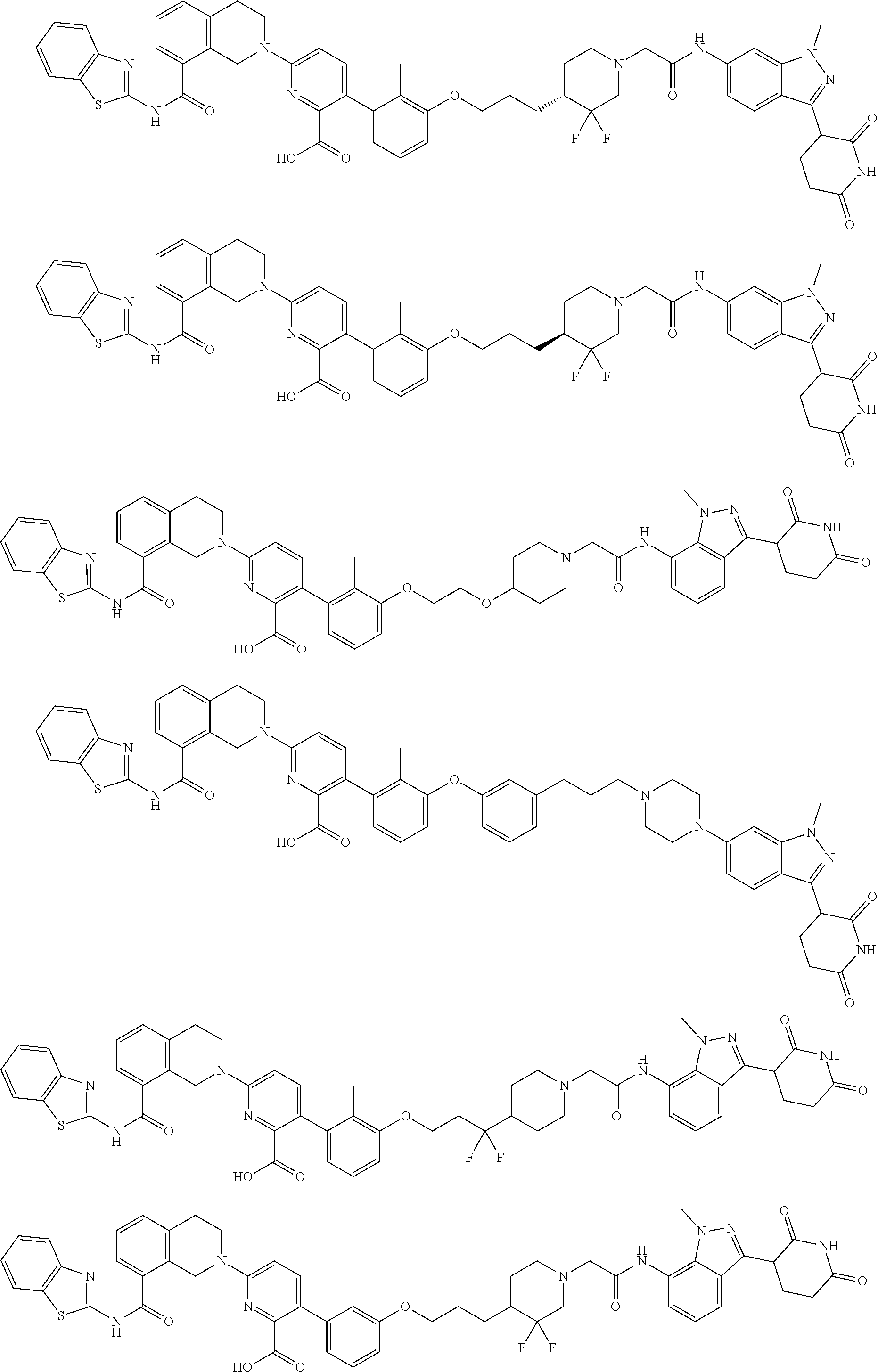 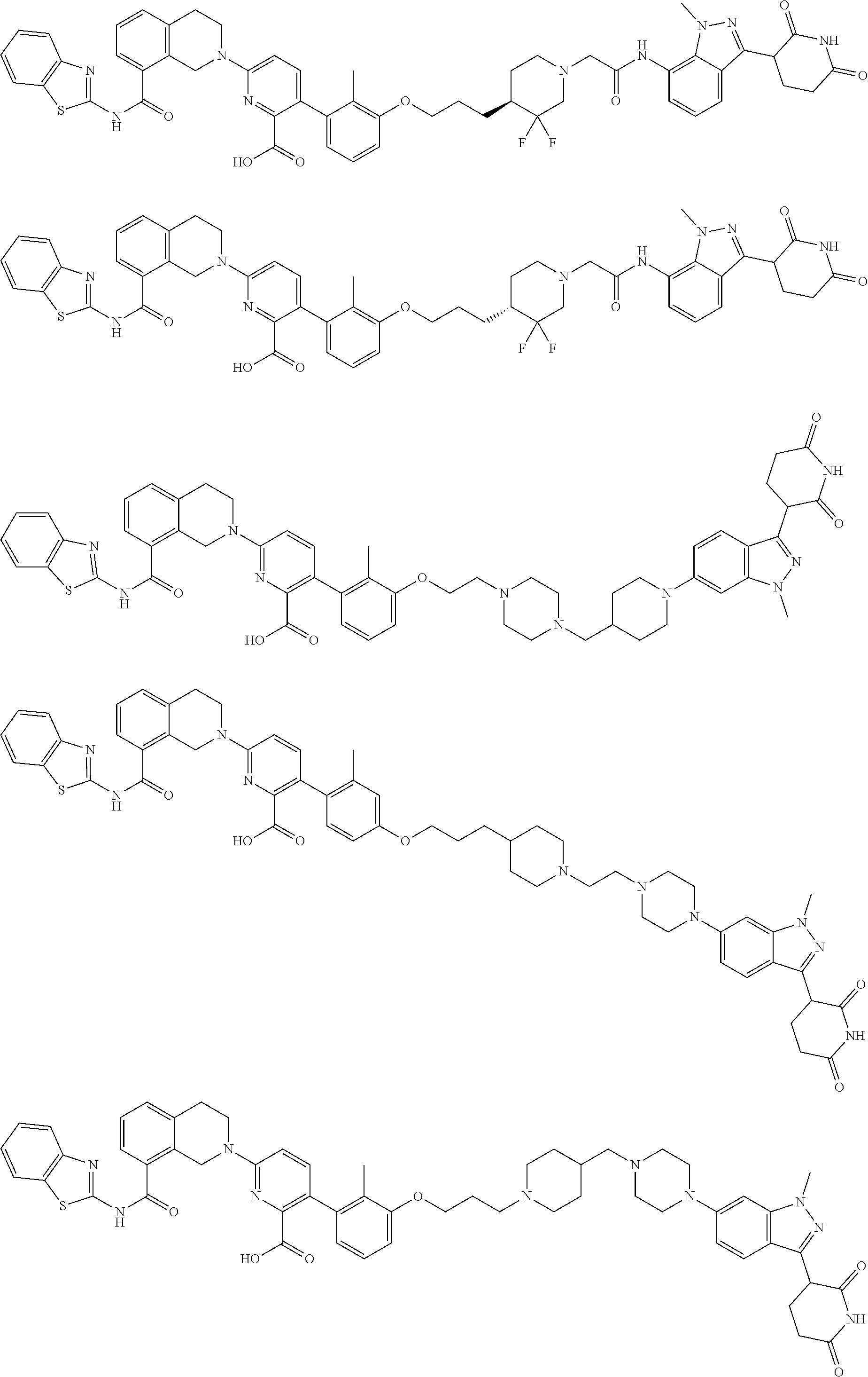 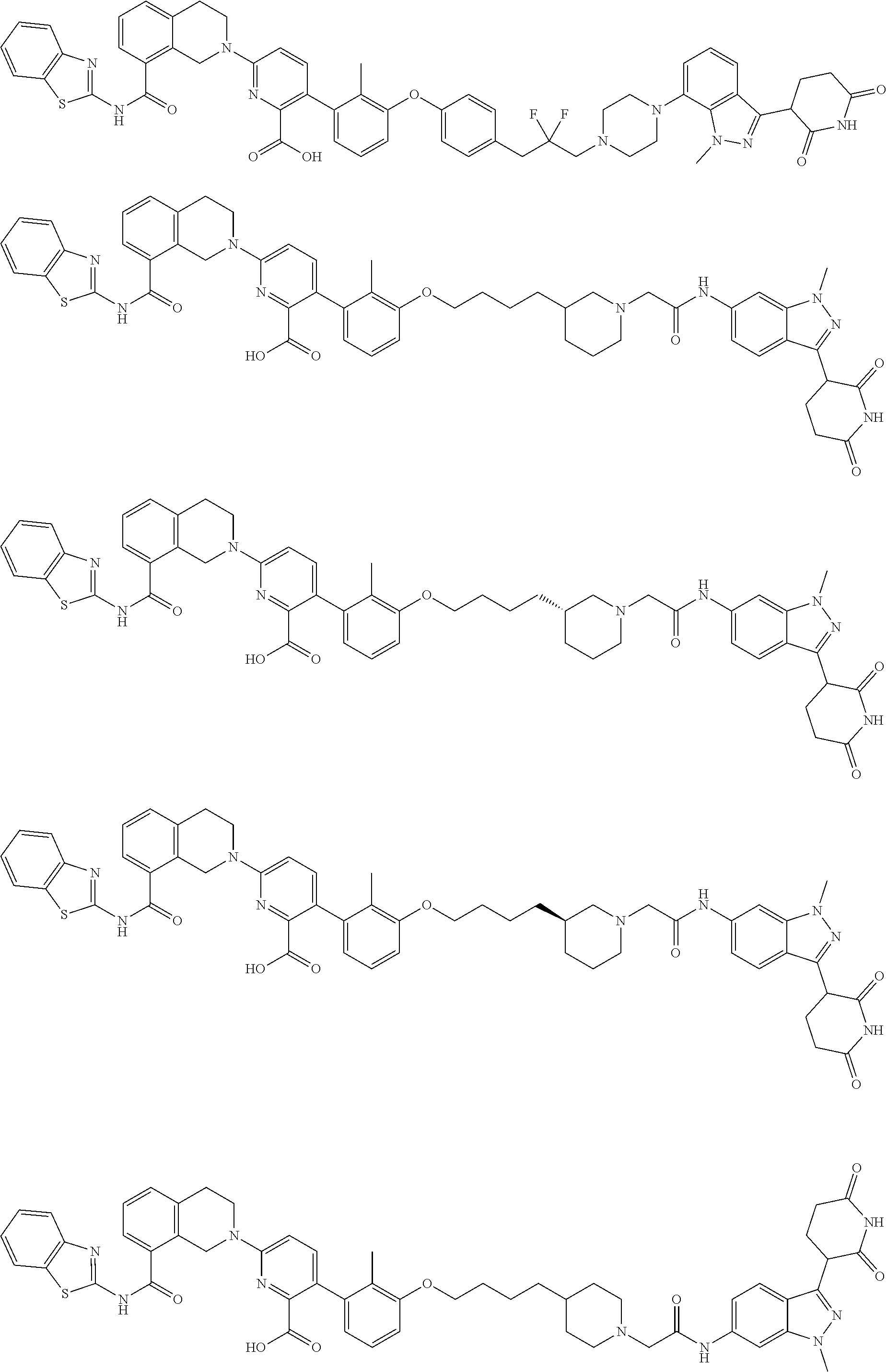 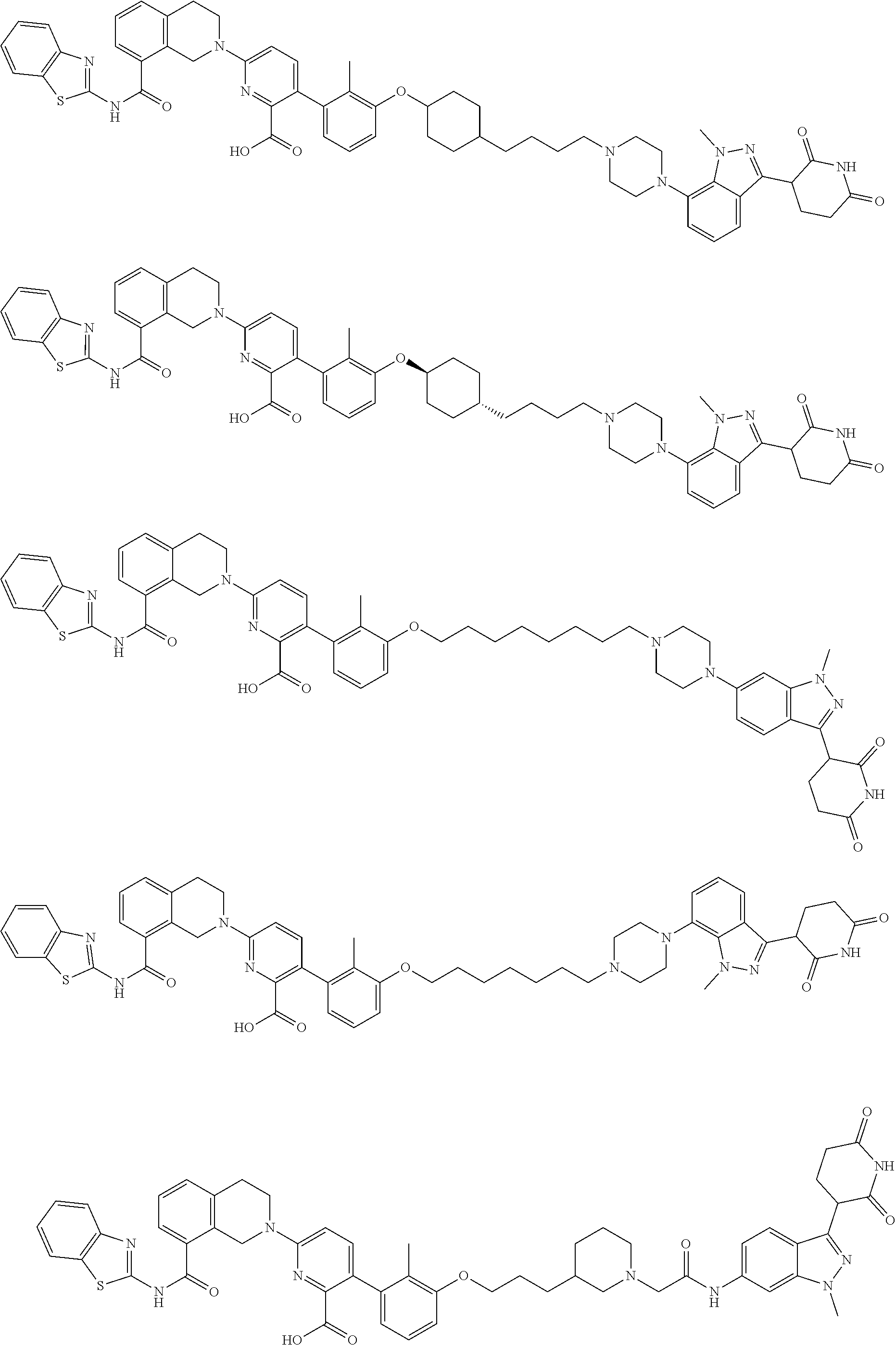 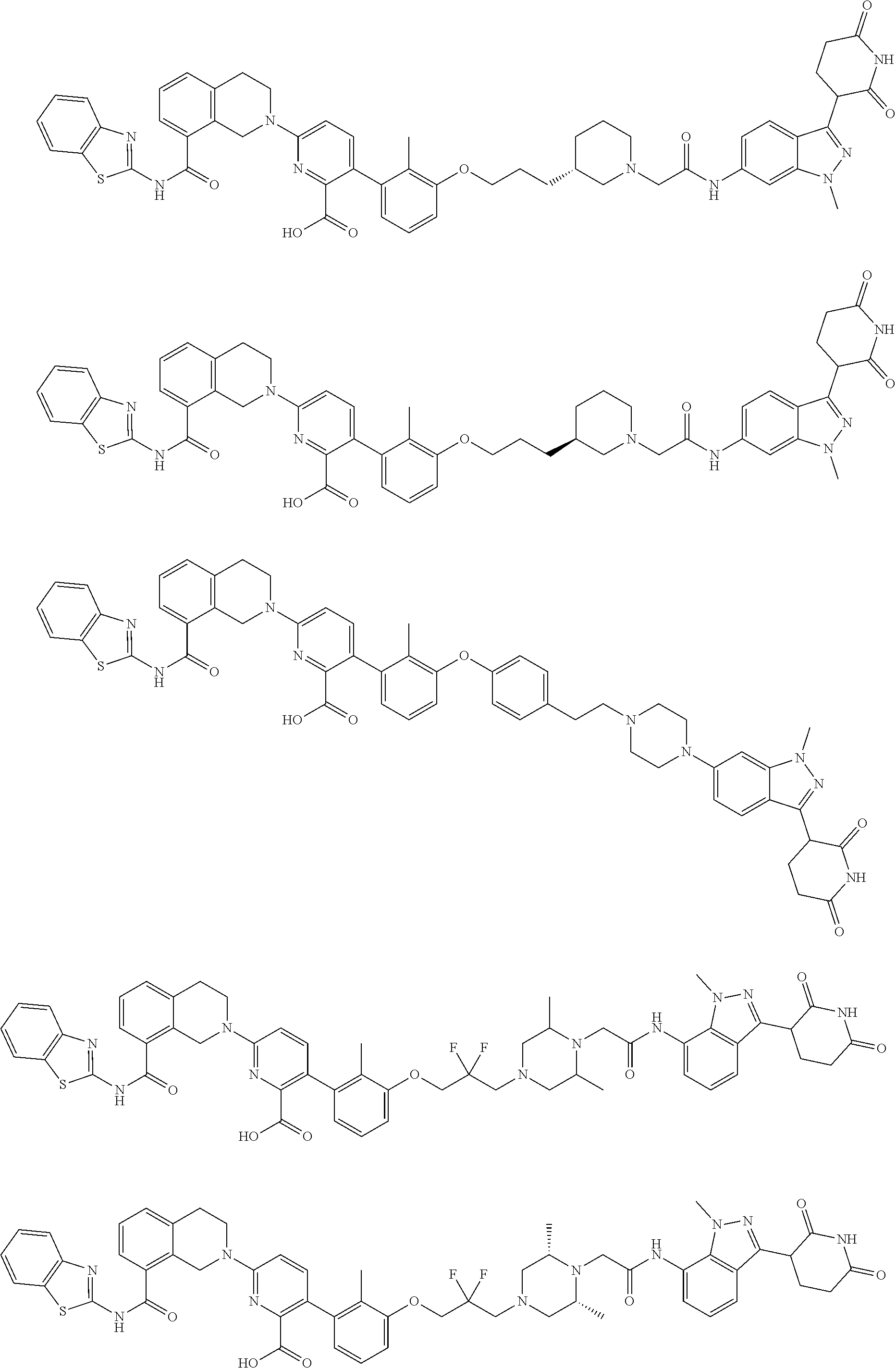 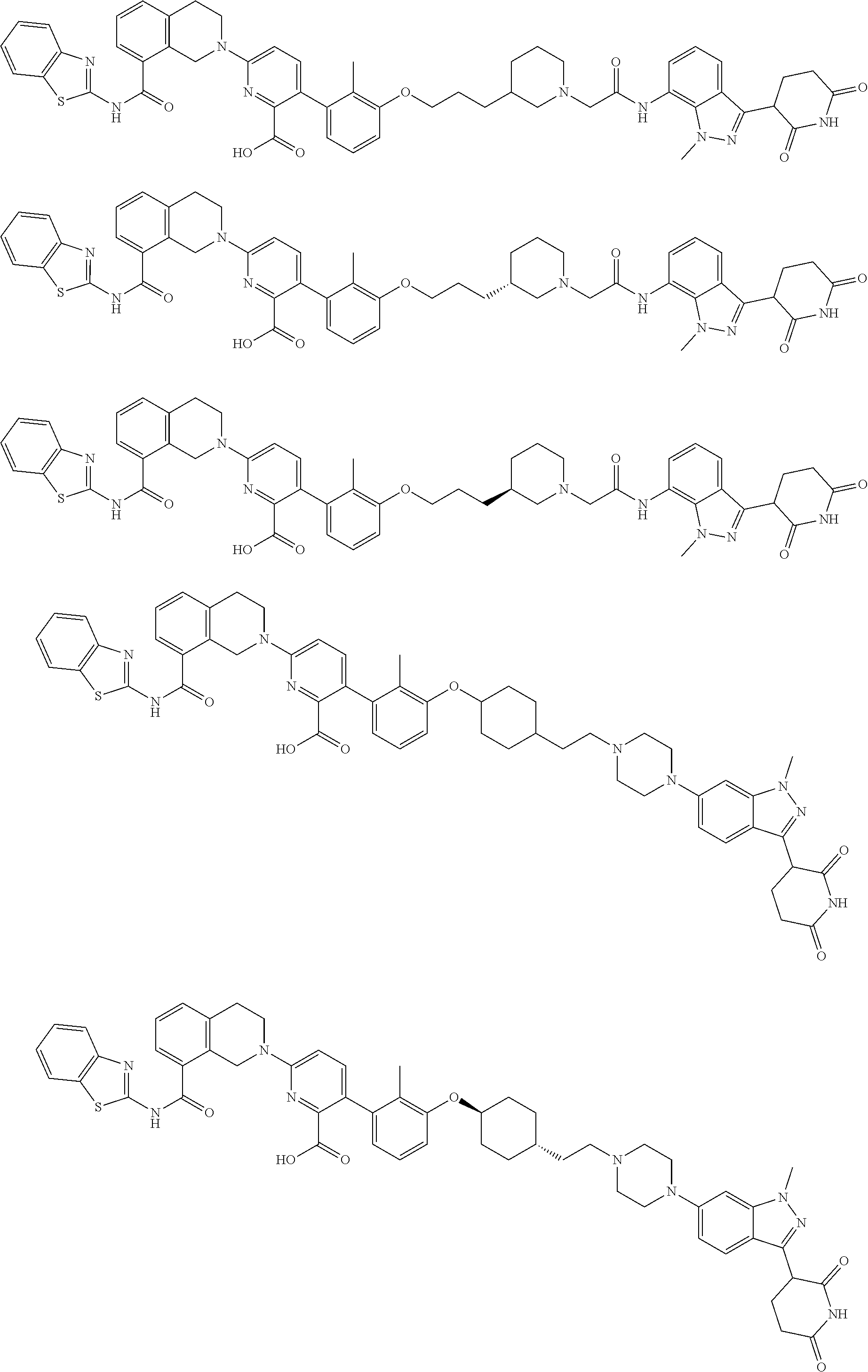 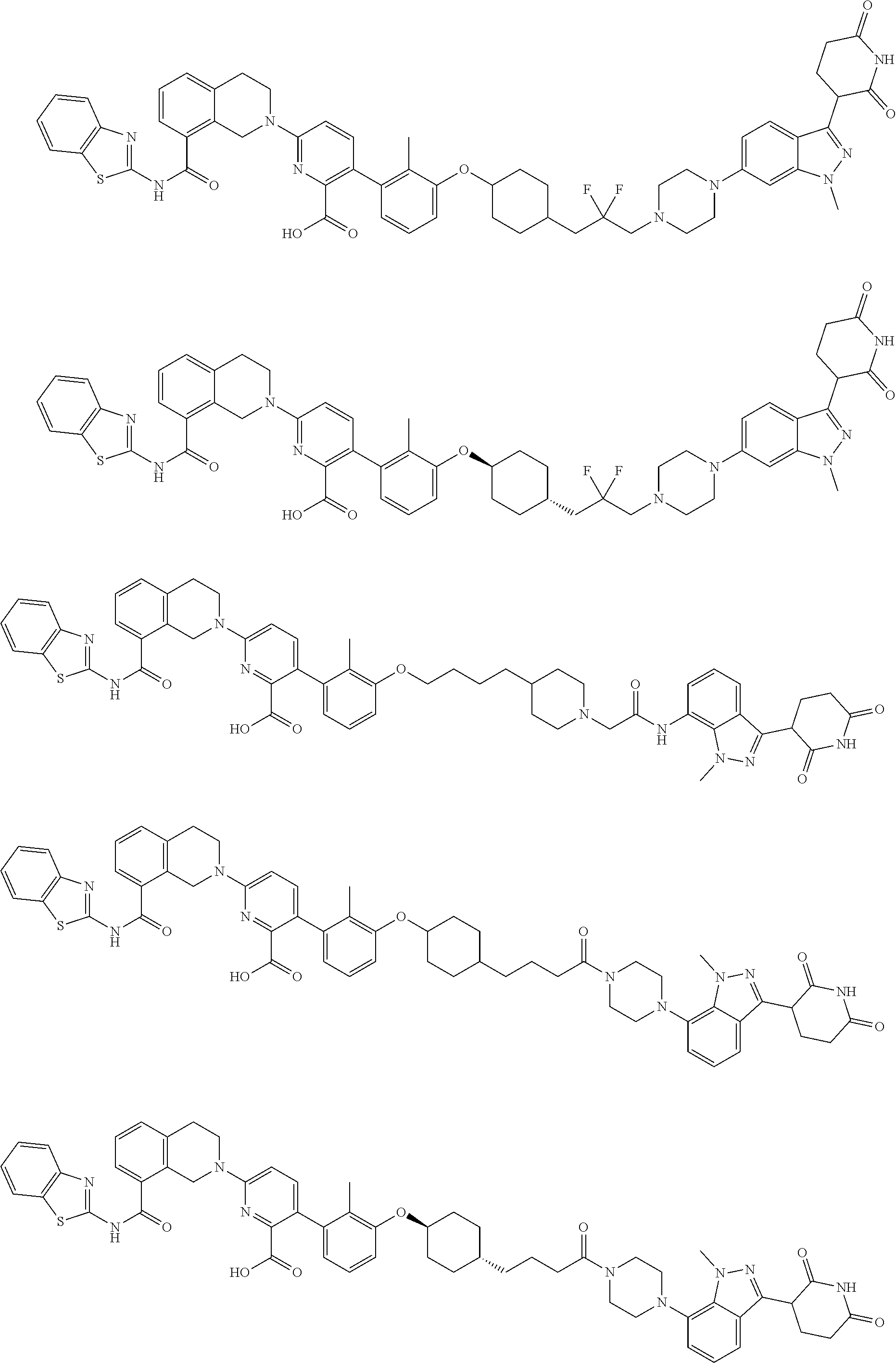 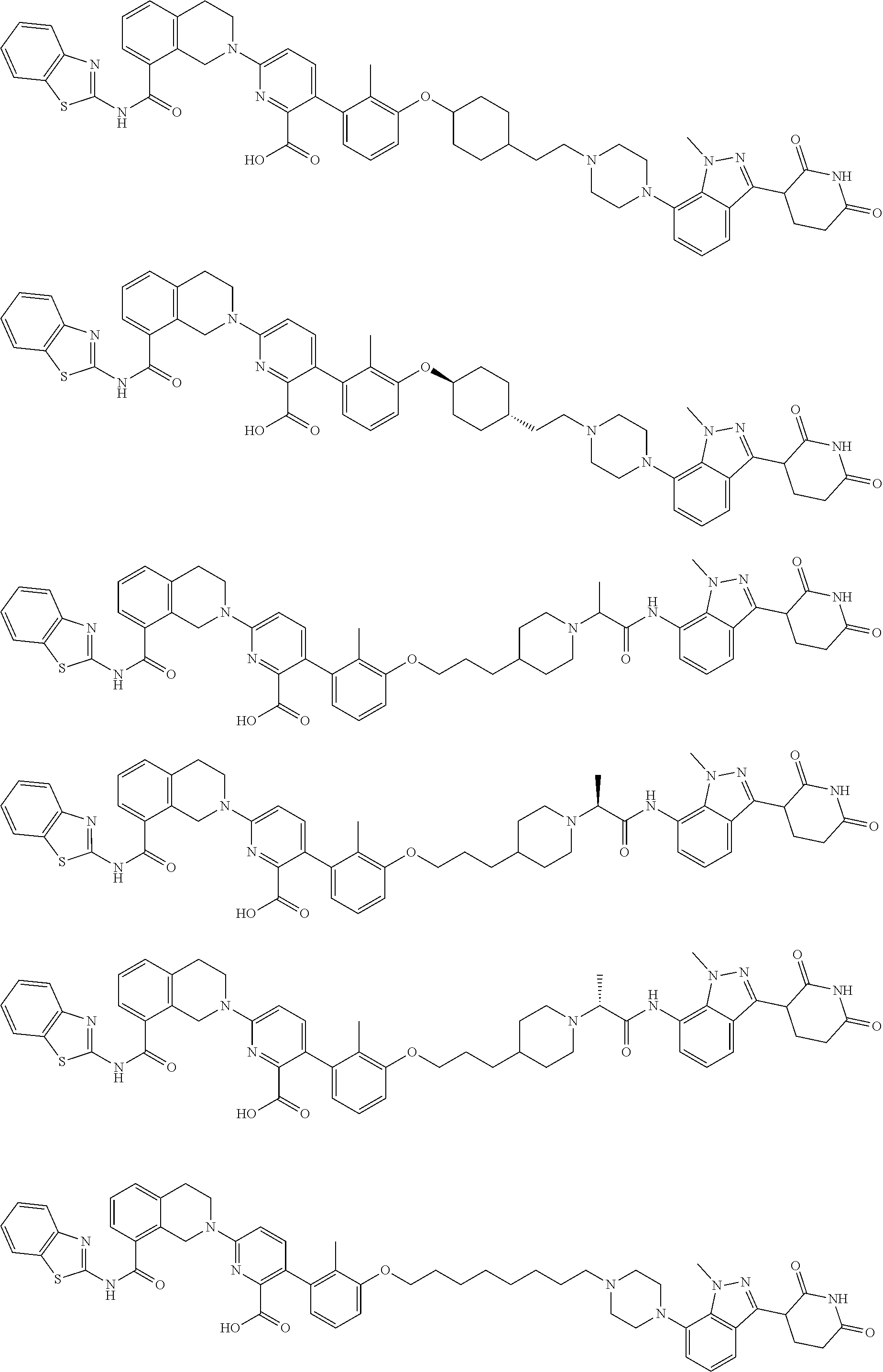 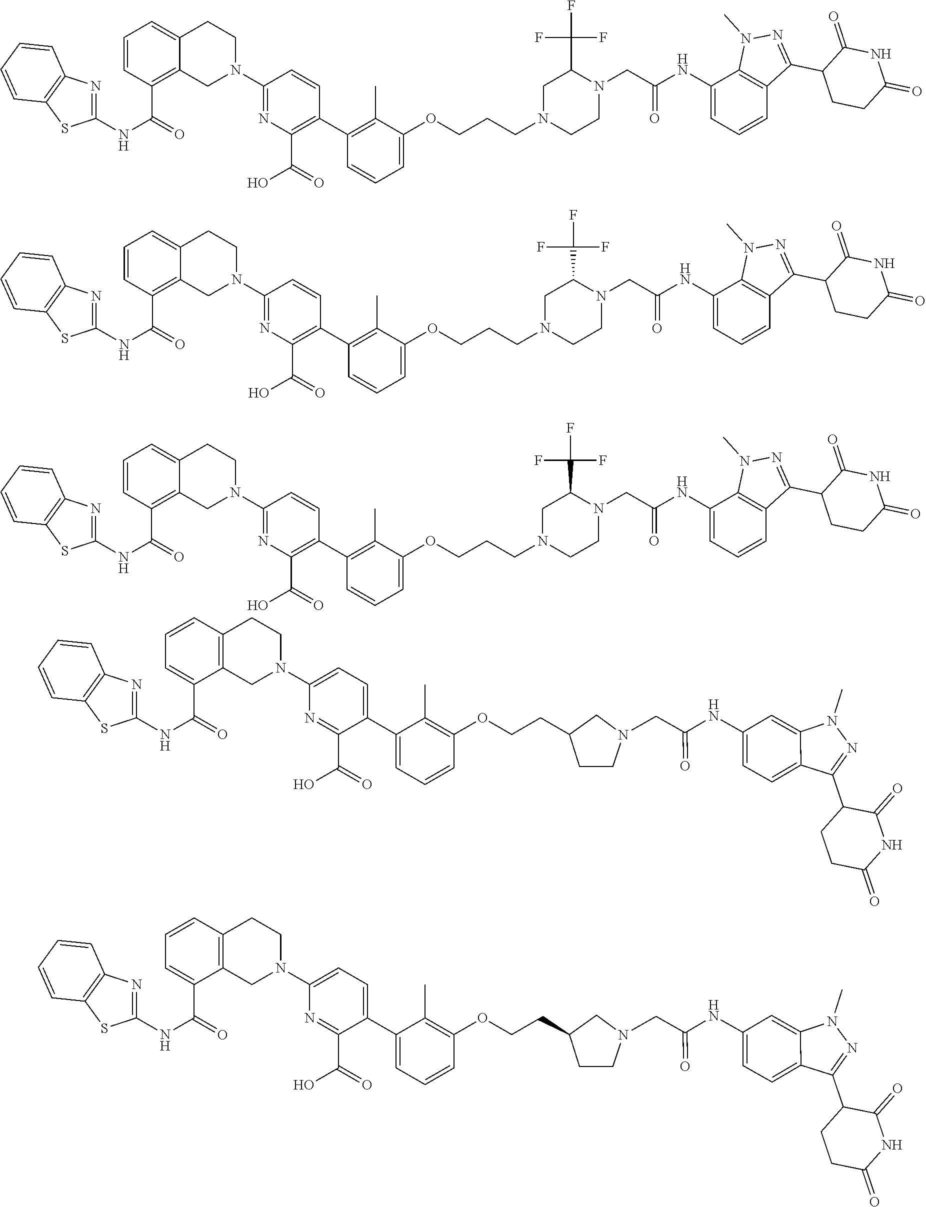 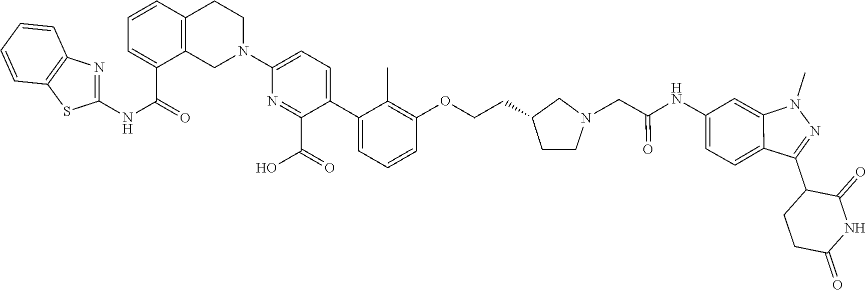 [SMILES: Cc1c(OCC[C@H]2CCN(CC(=O)Nc3ccc4c(C5CCC(=O)NC5=O)nn(C)c4c3)C2)cccc1-c1ccc(N2CCc3cccc(C(=O)Nc4nc5ccccc5s4)c3C2)nc1C(=O)O] 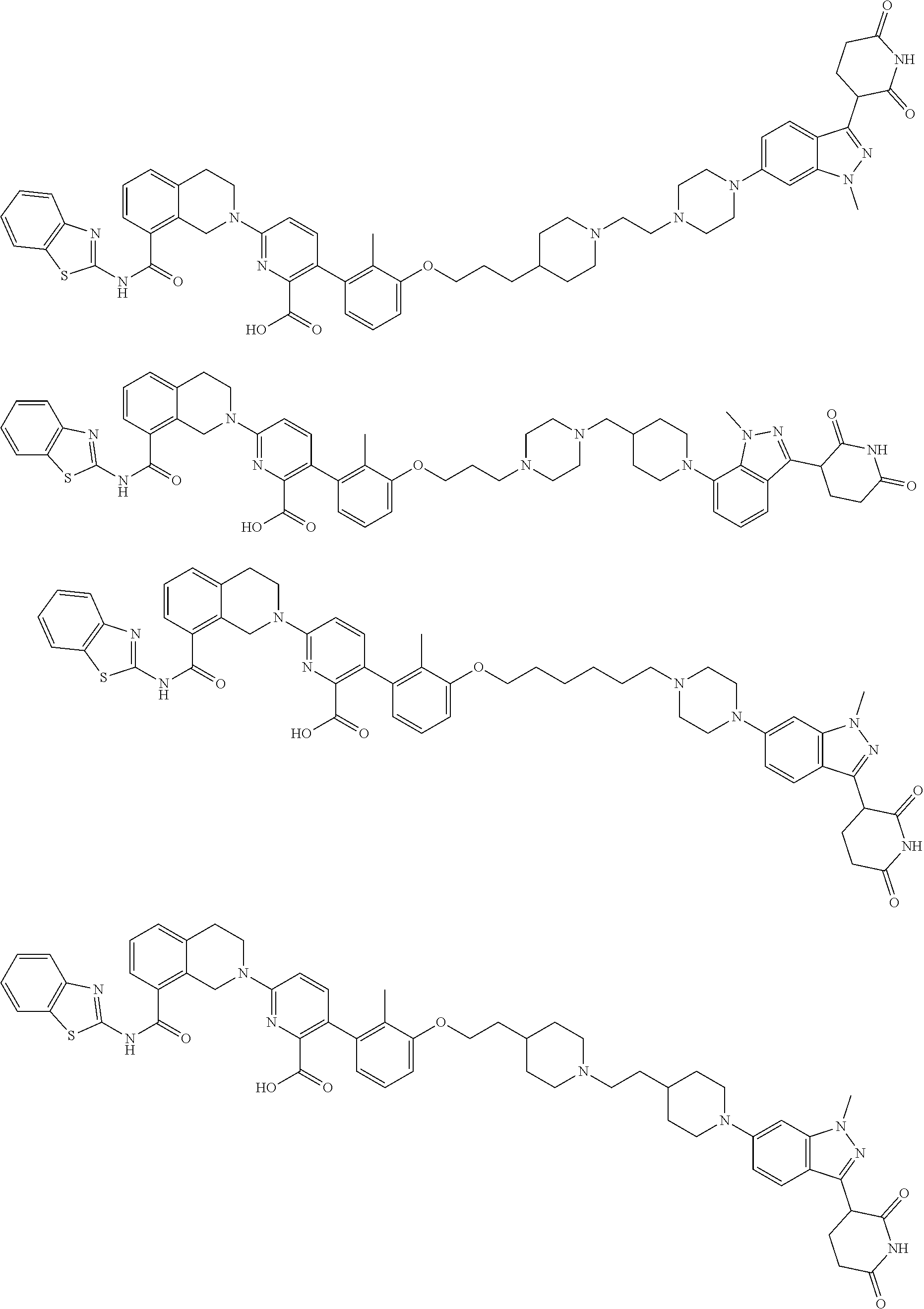 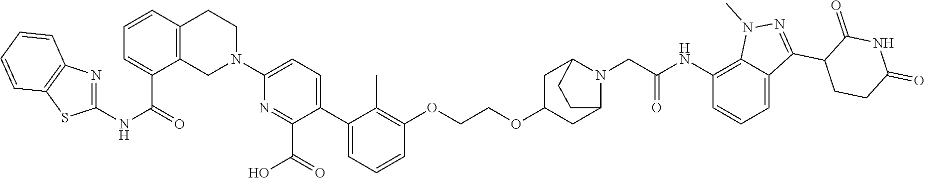 [SMILES: Cc1c(OCCOC2CC3CCC(C2)N3CC(=O)Nc2cccc3c(C4CCC(=O)NC4=O)nn(C)c23)cccc1-c1ccc(N2CCc3cccc(C(=O)Nc4nc5ccccc5s4)c3C2)nc1C(=O)O] 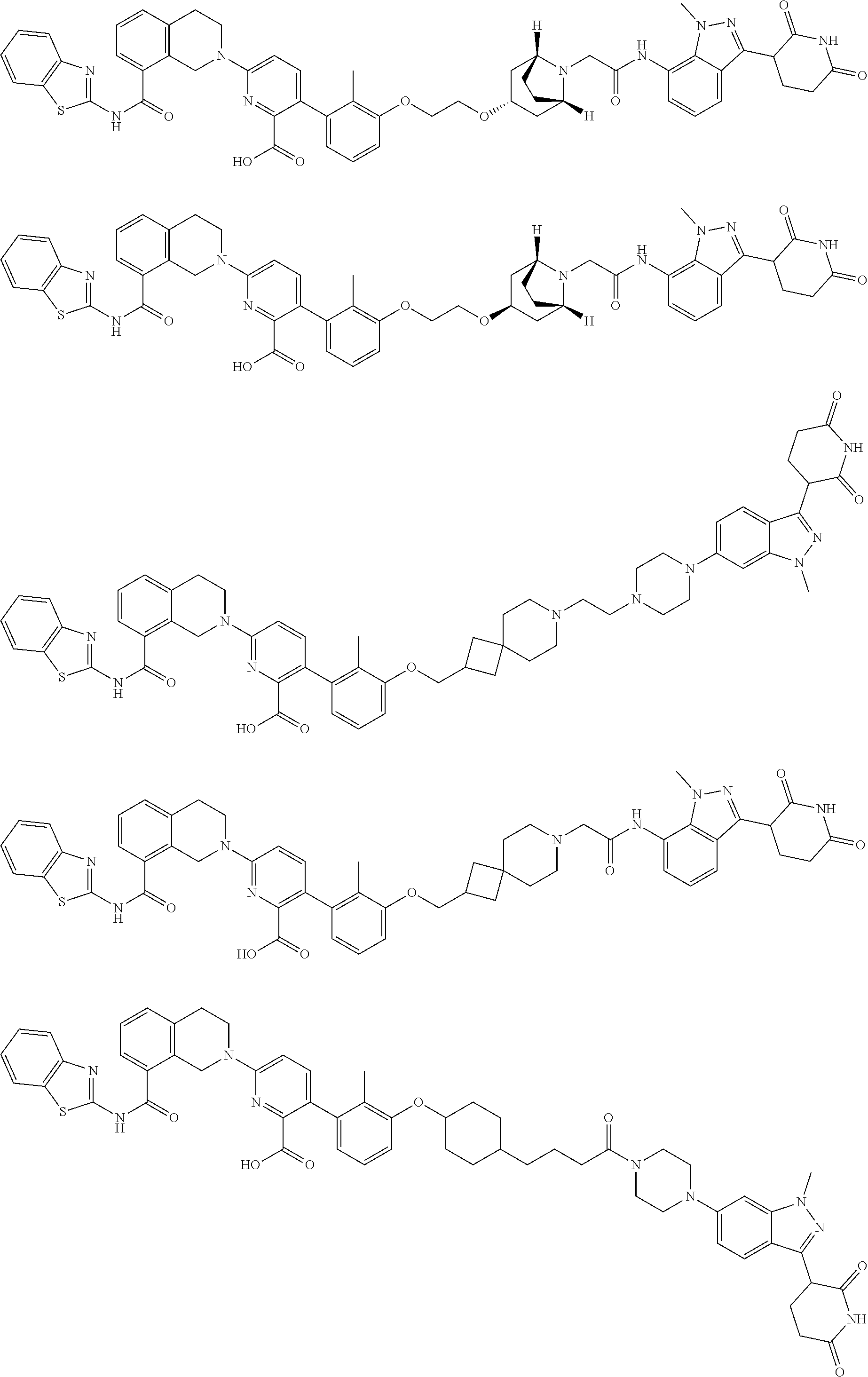 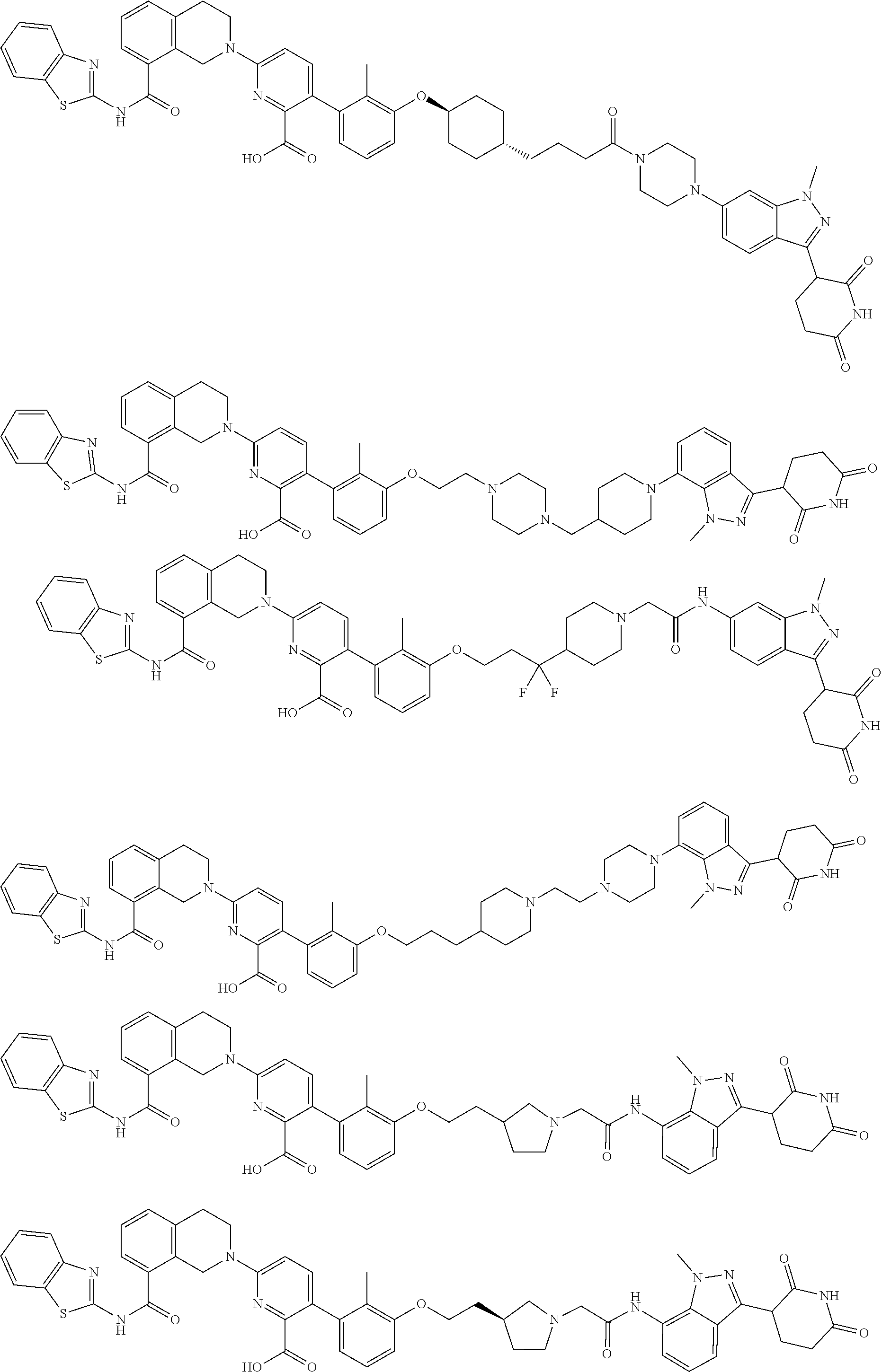 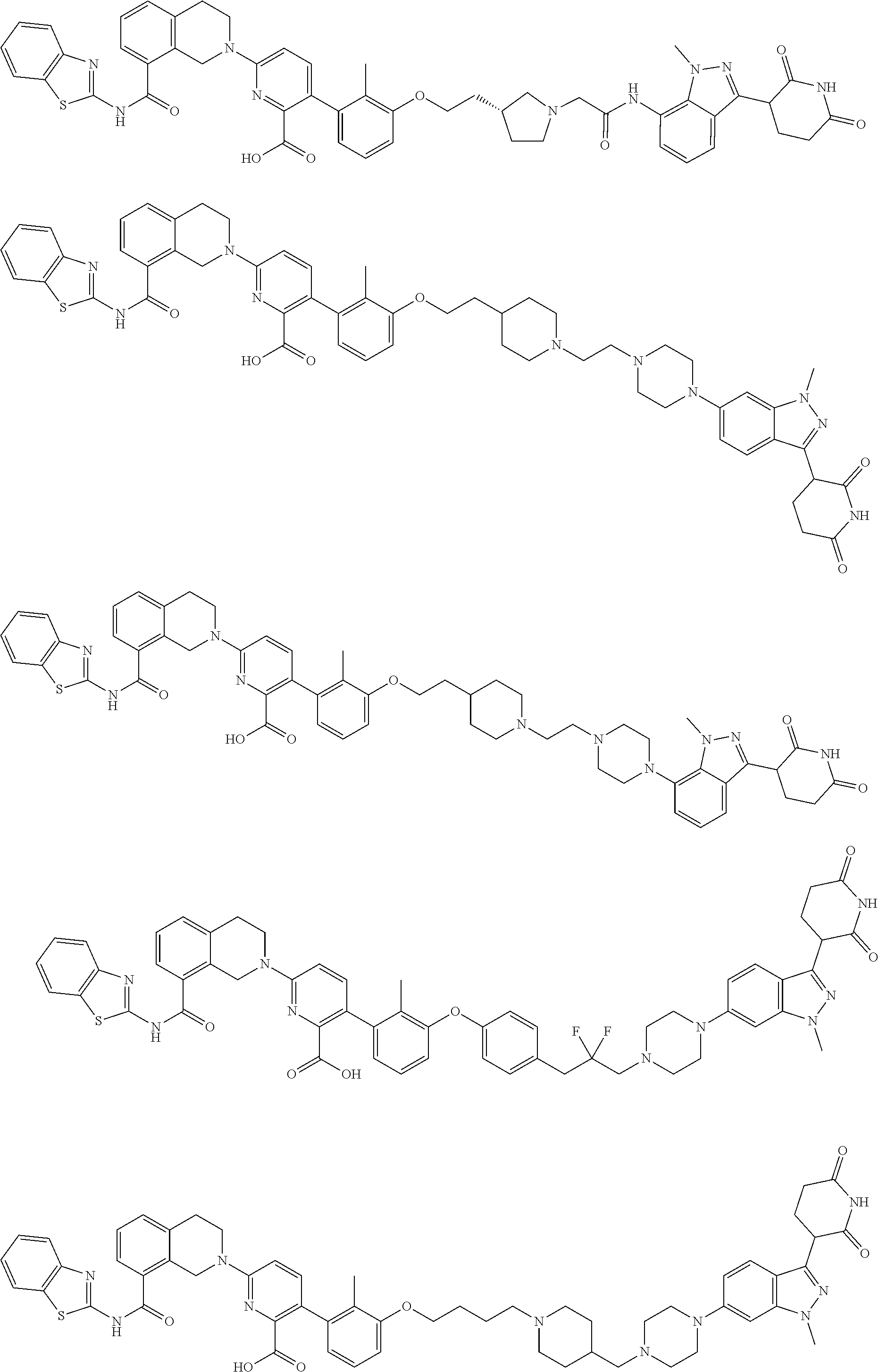 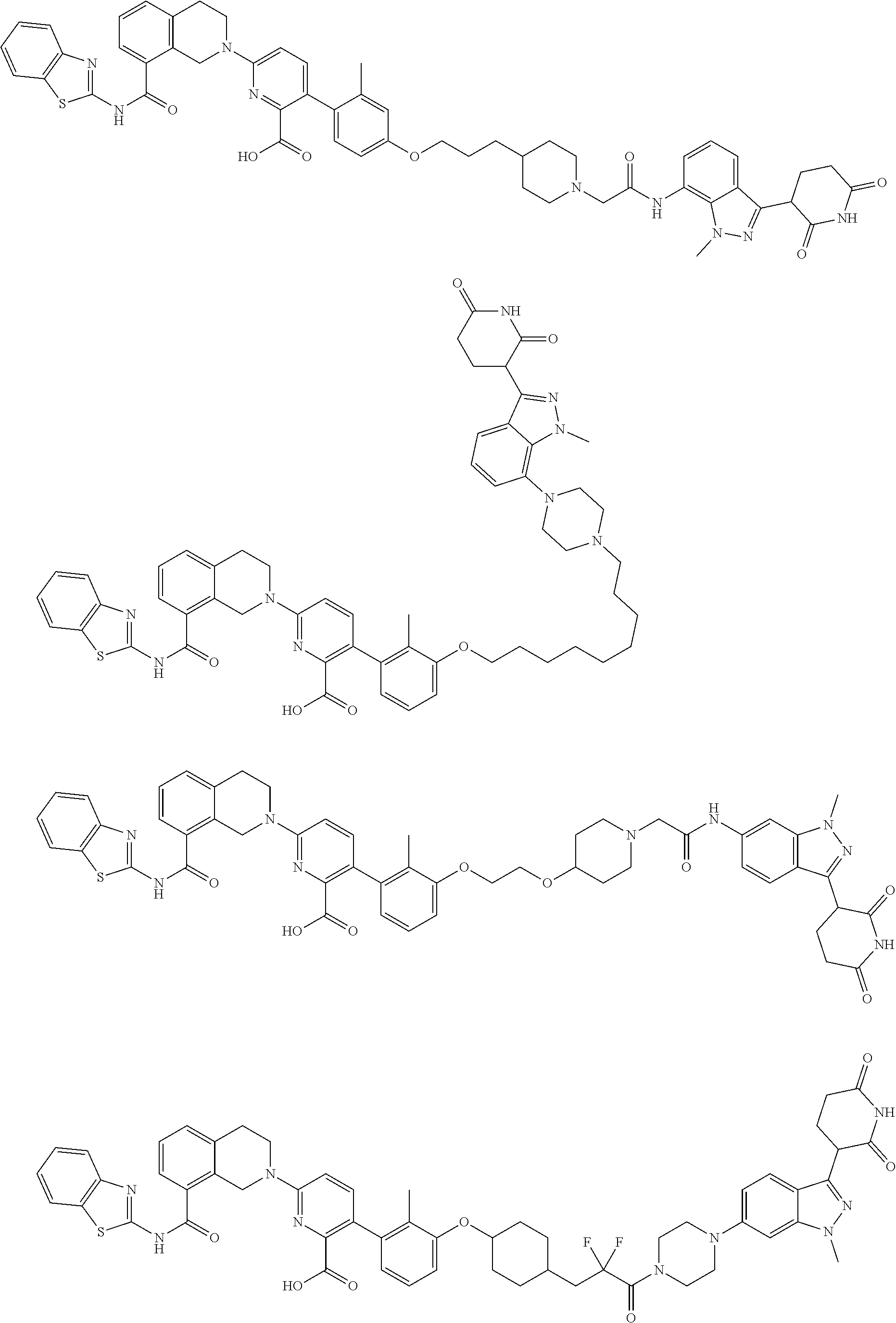 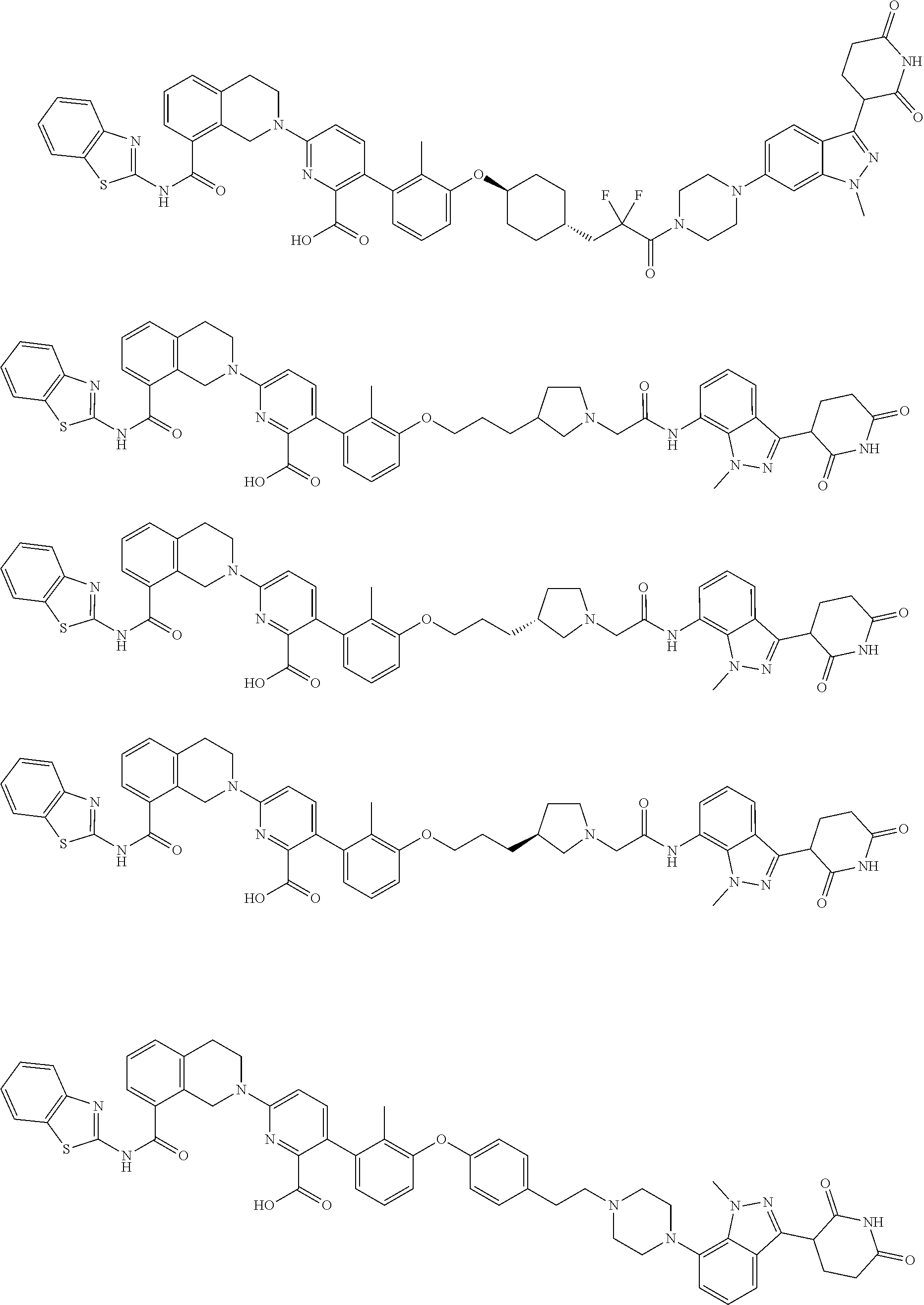 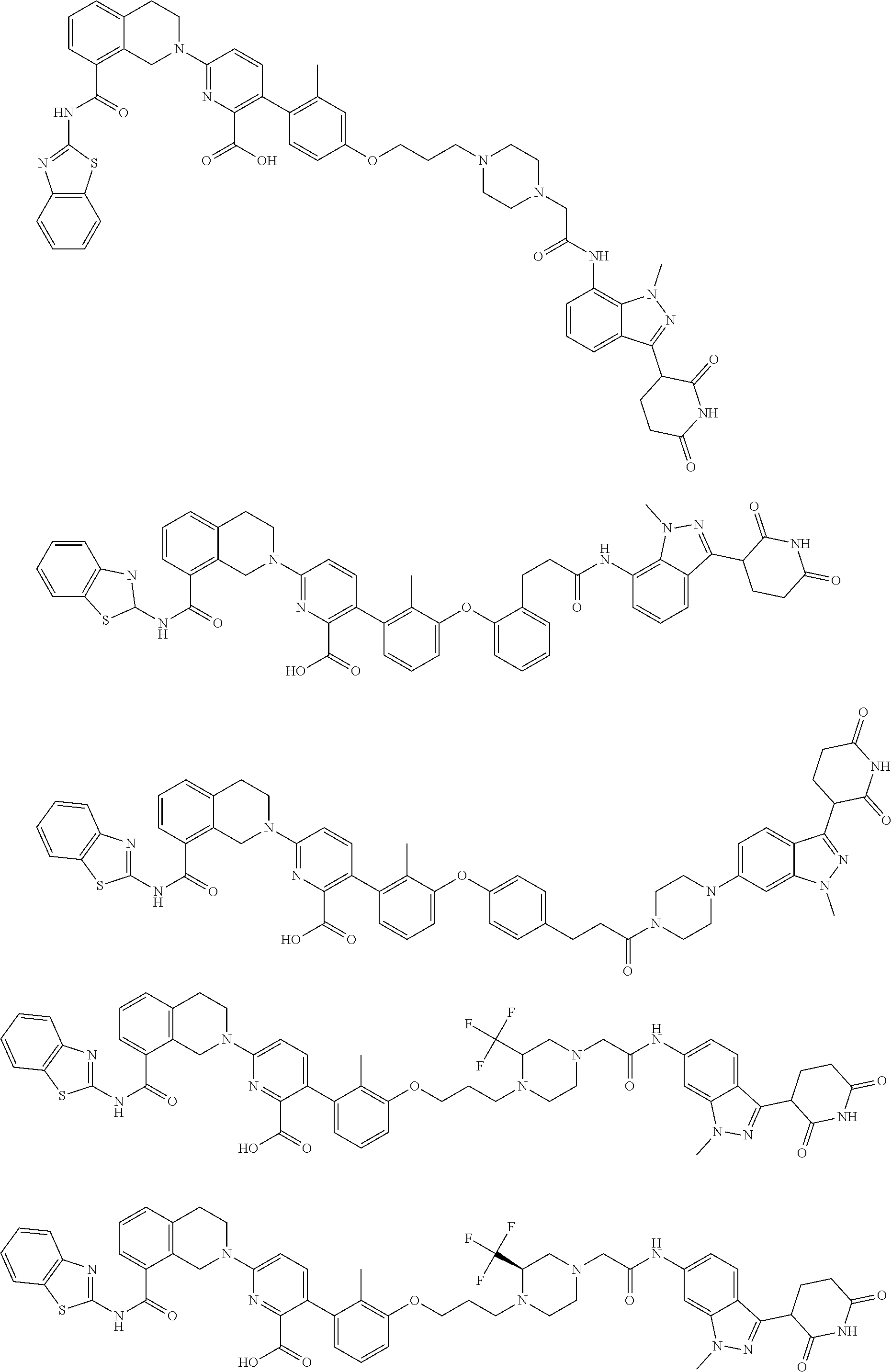 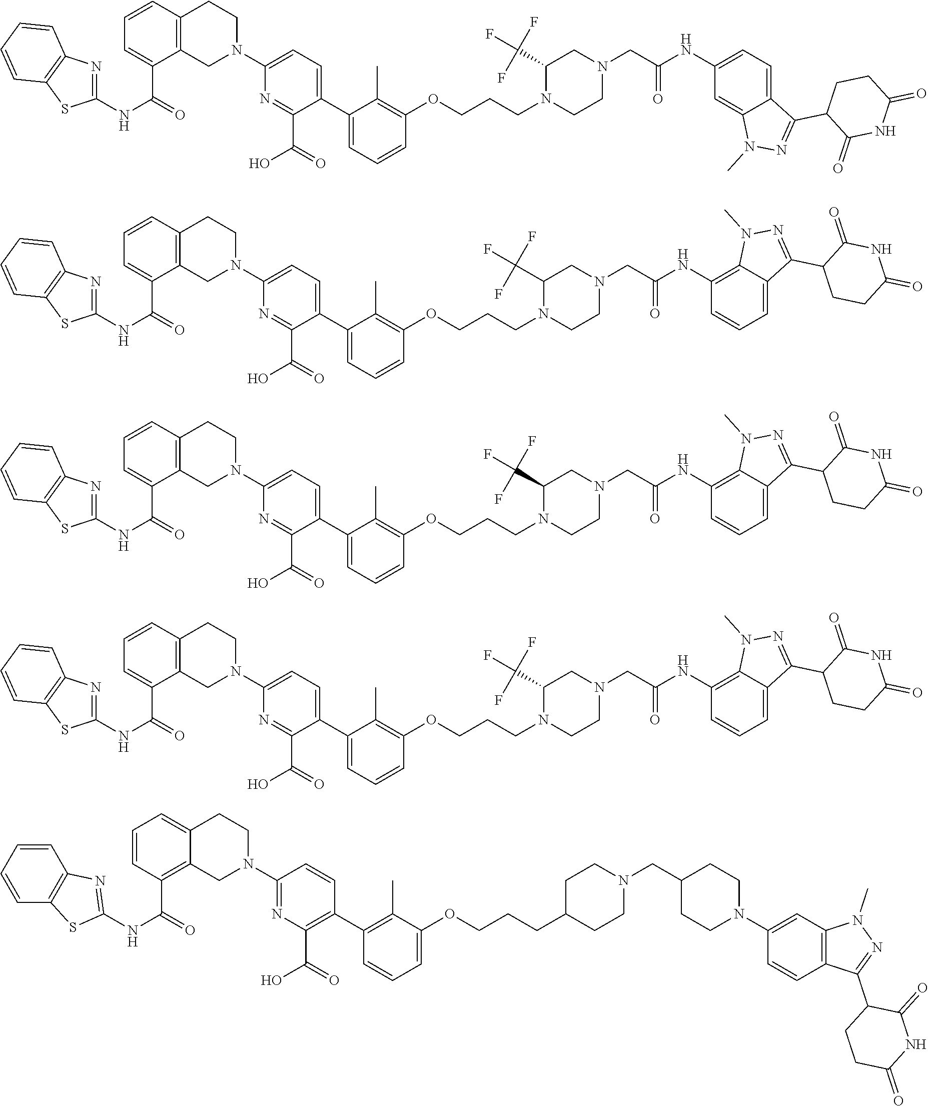 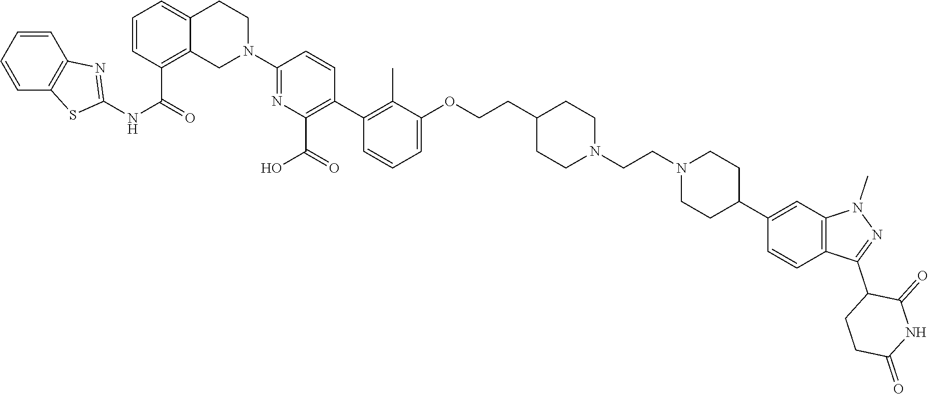 [SMILES: Cc1c(OCCC2CCN(CCN3CCC(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] 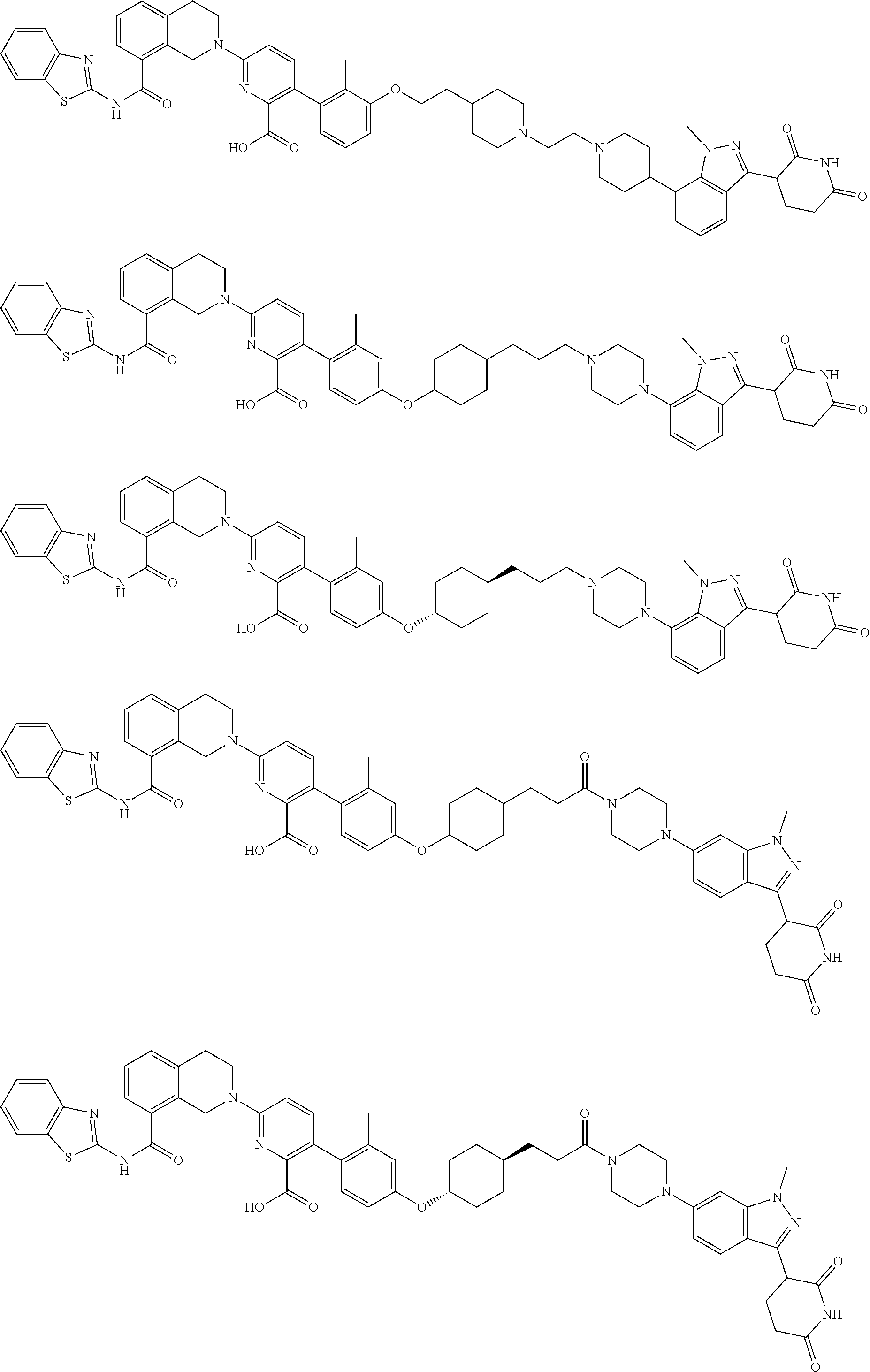 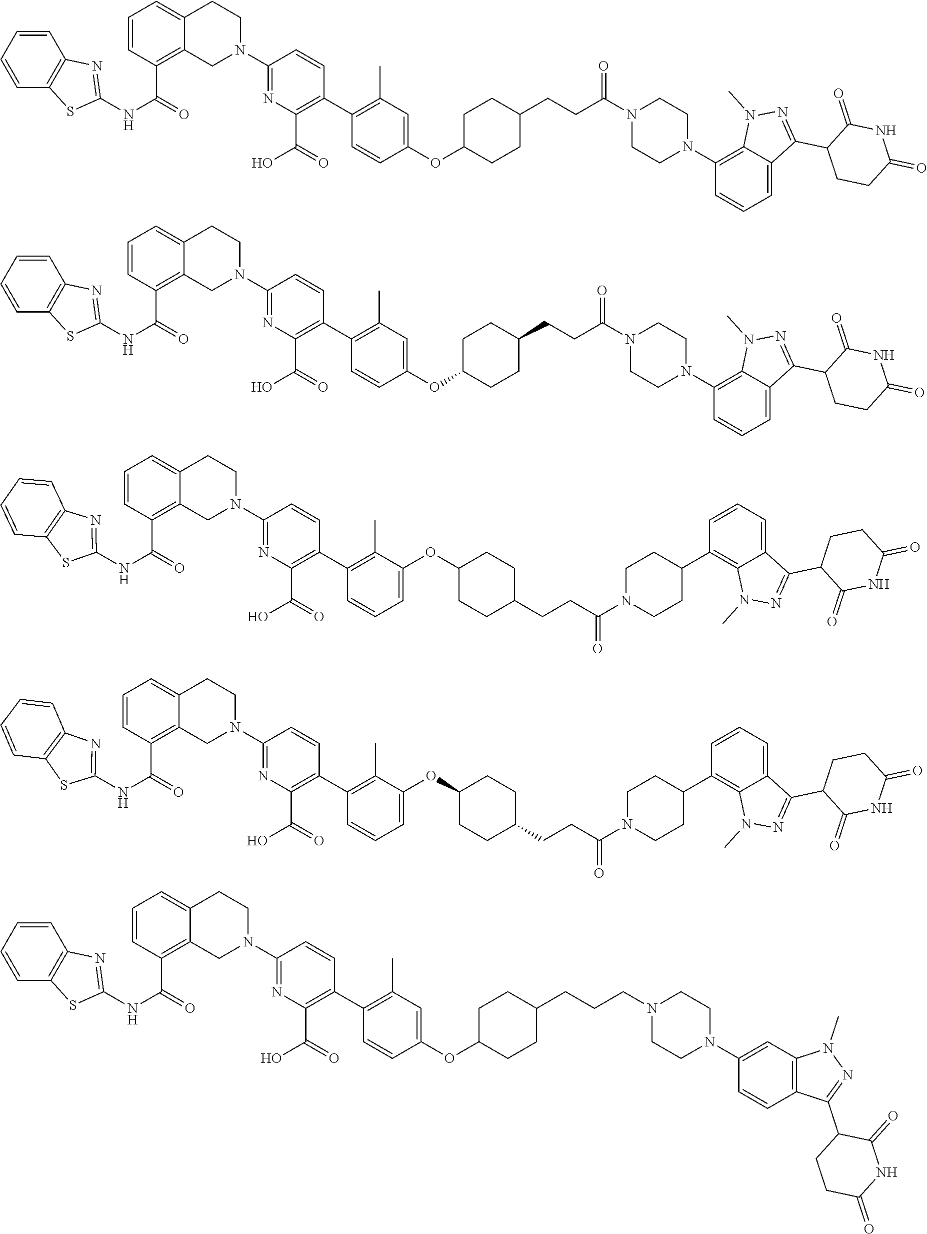 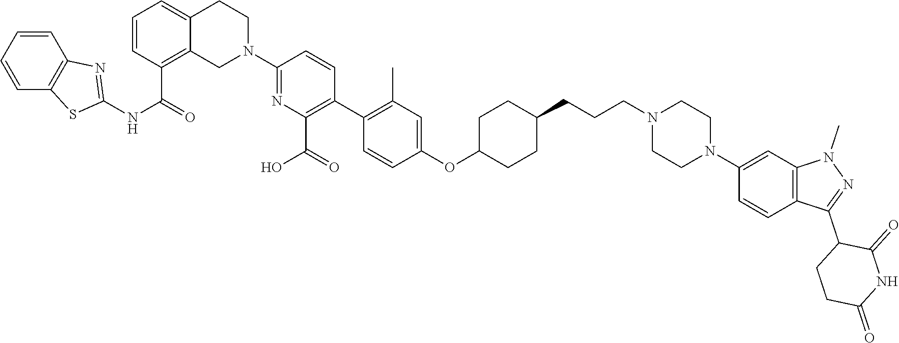 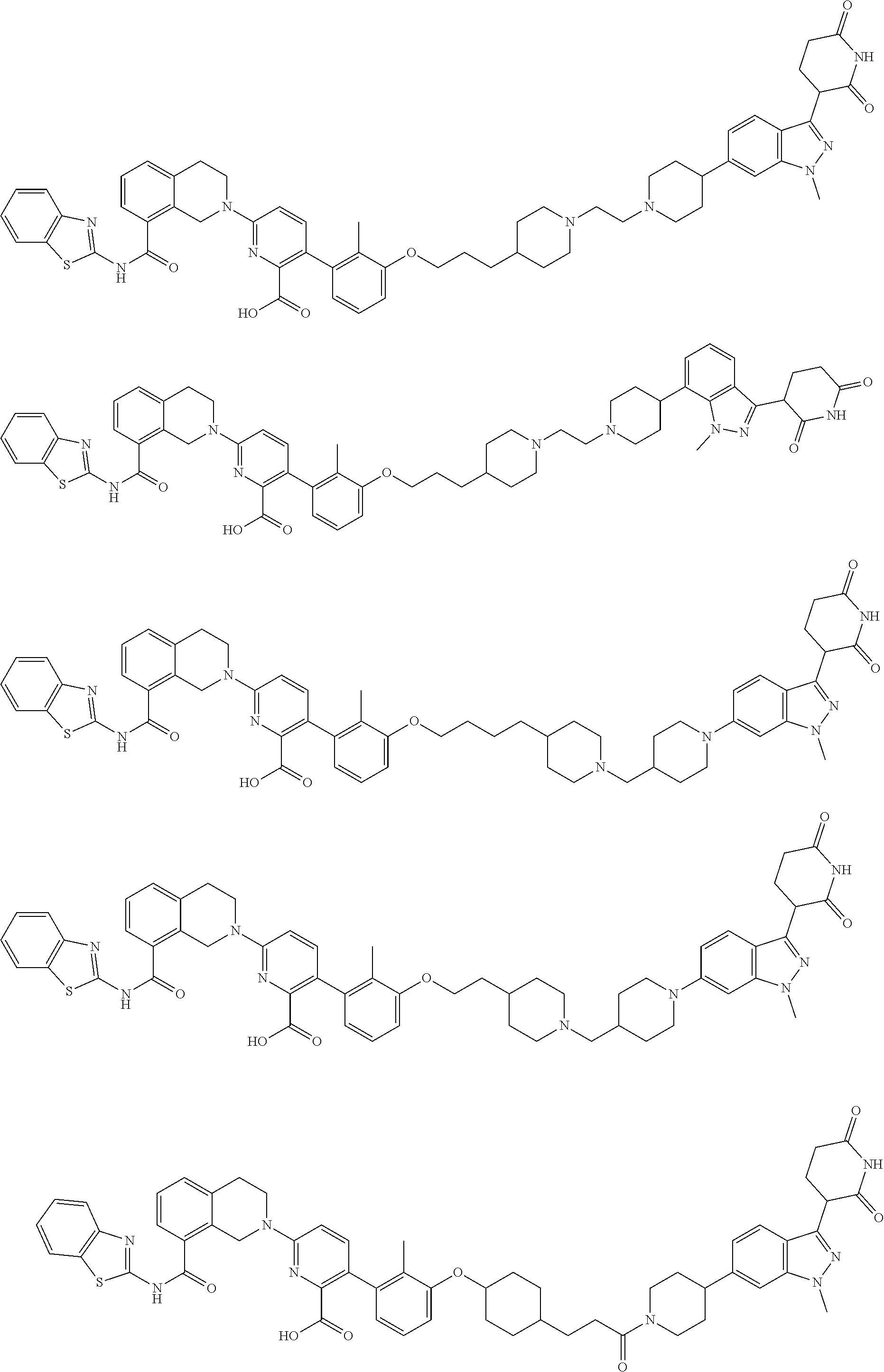 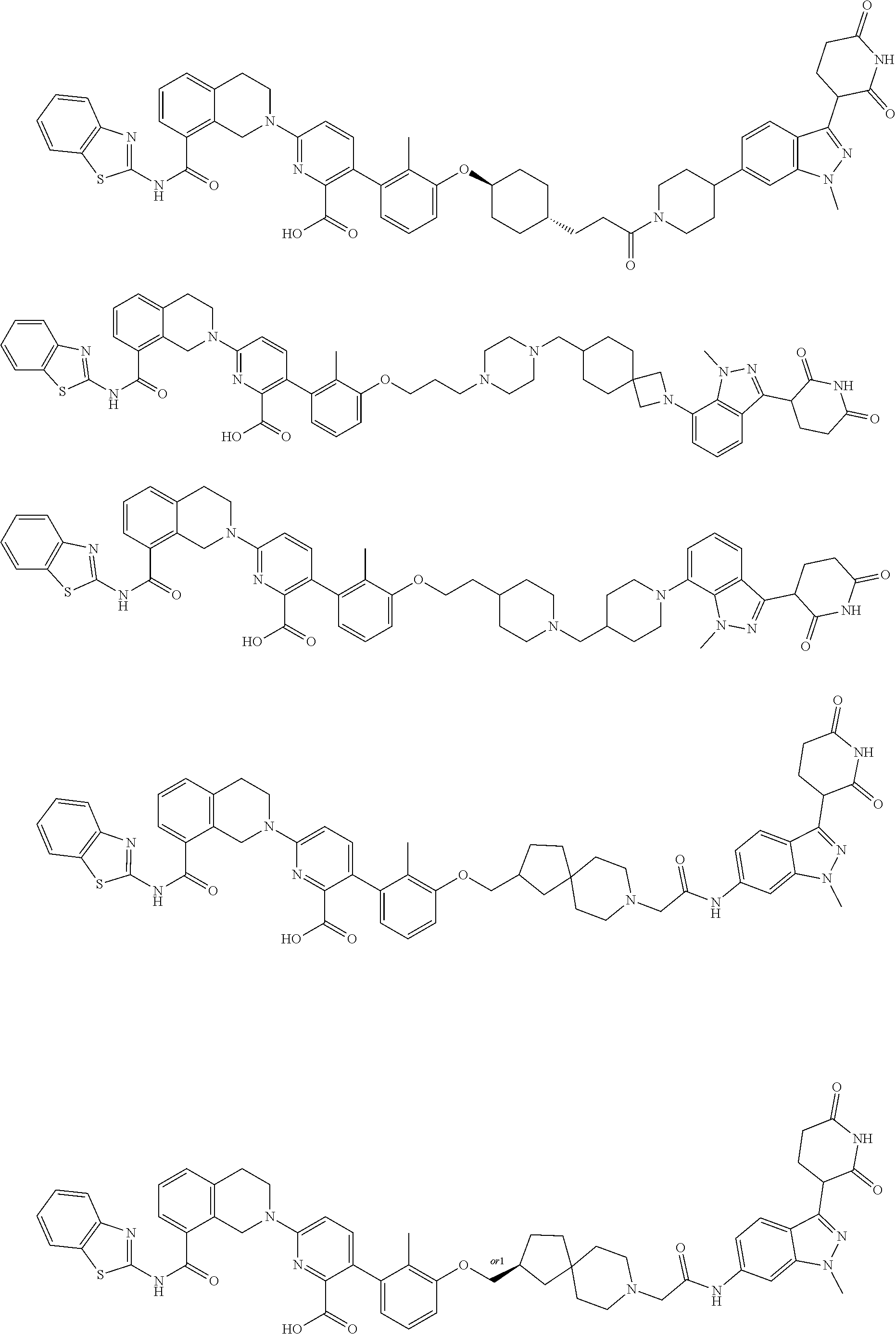 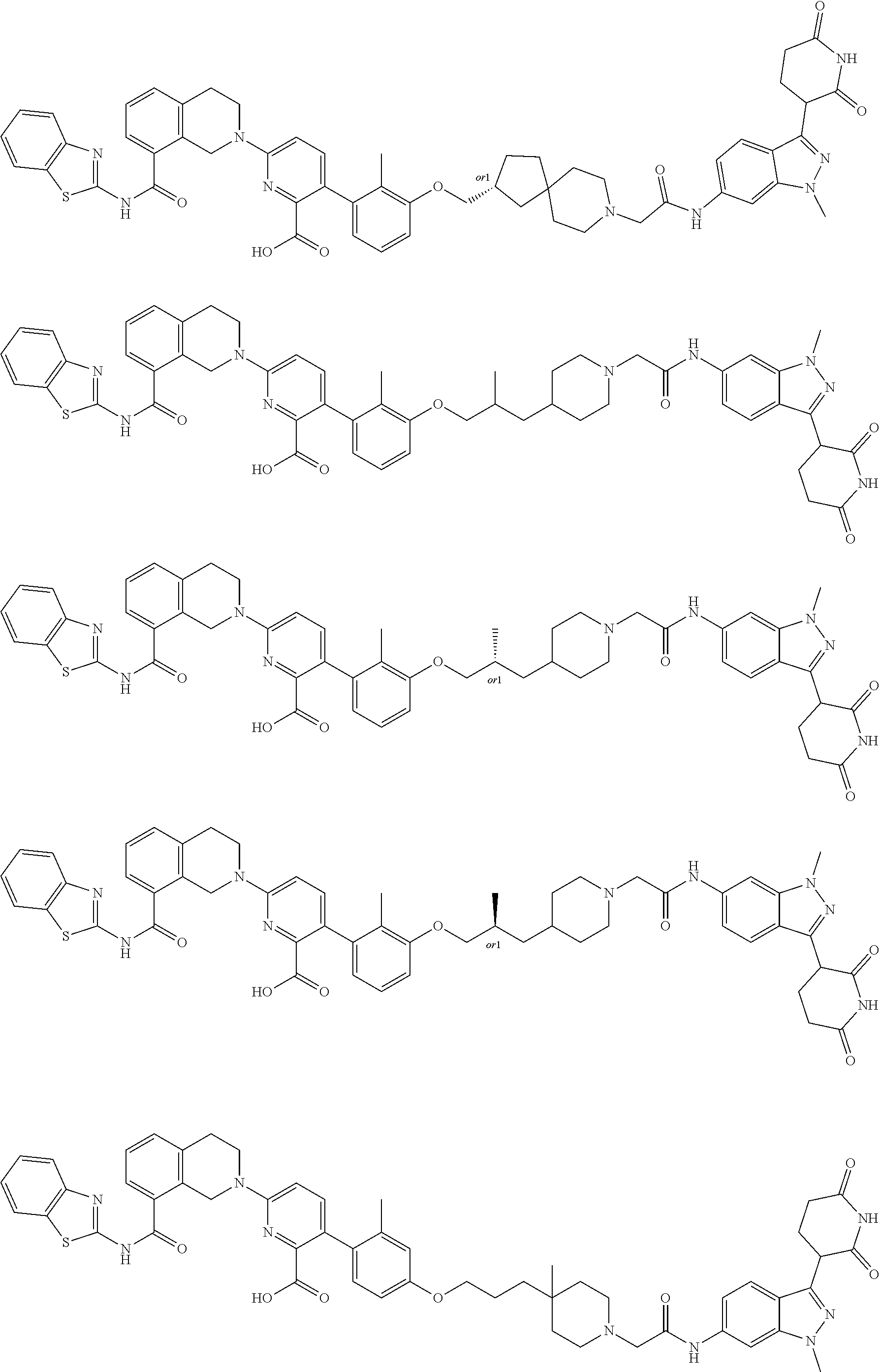 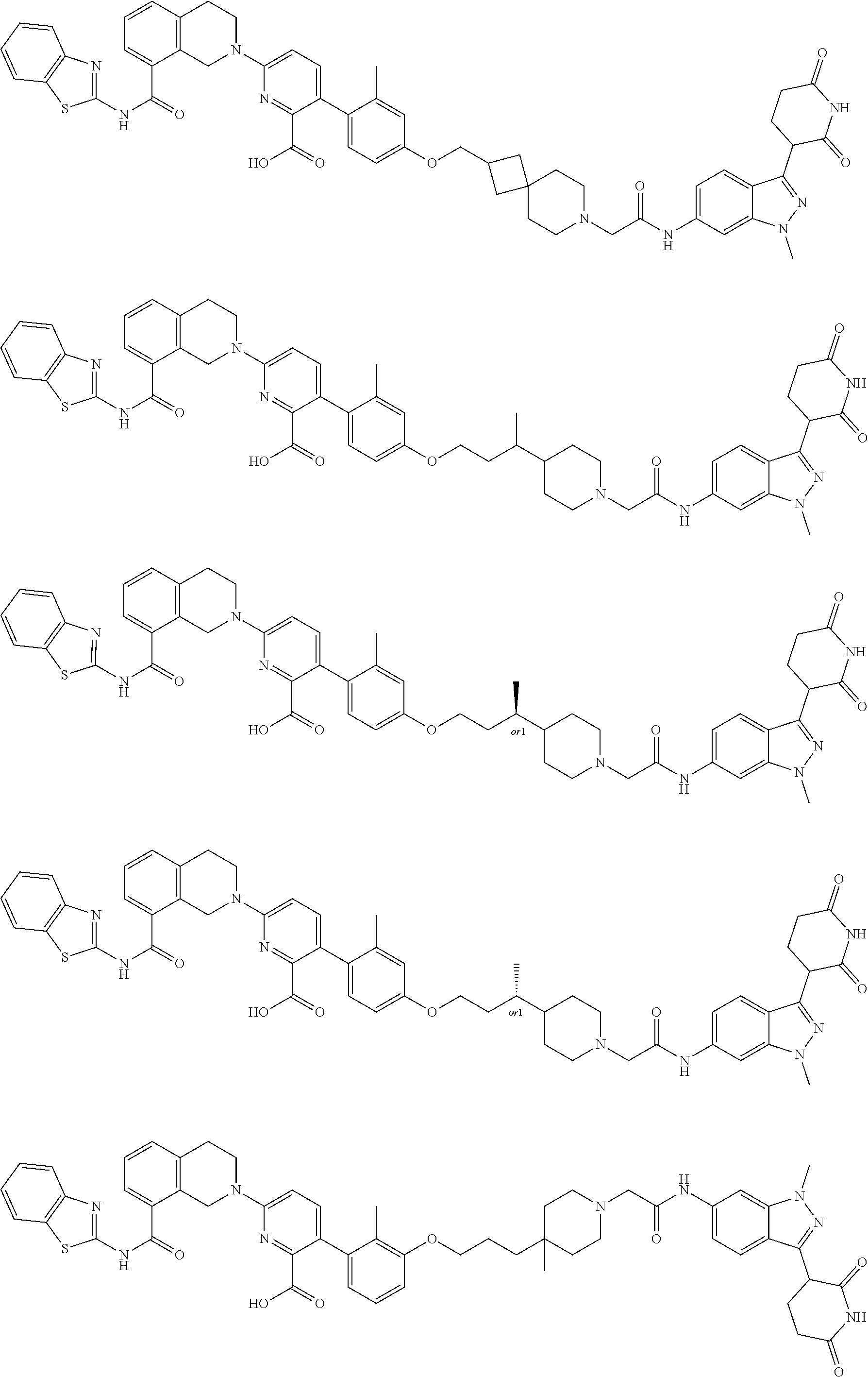 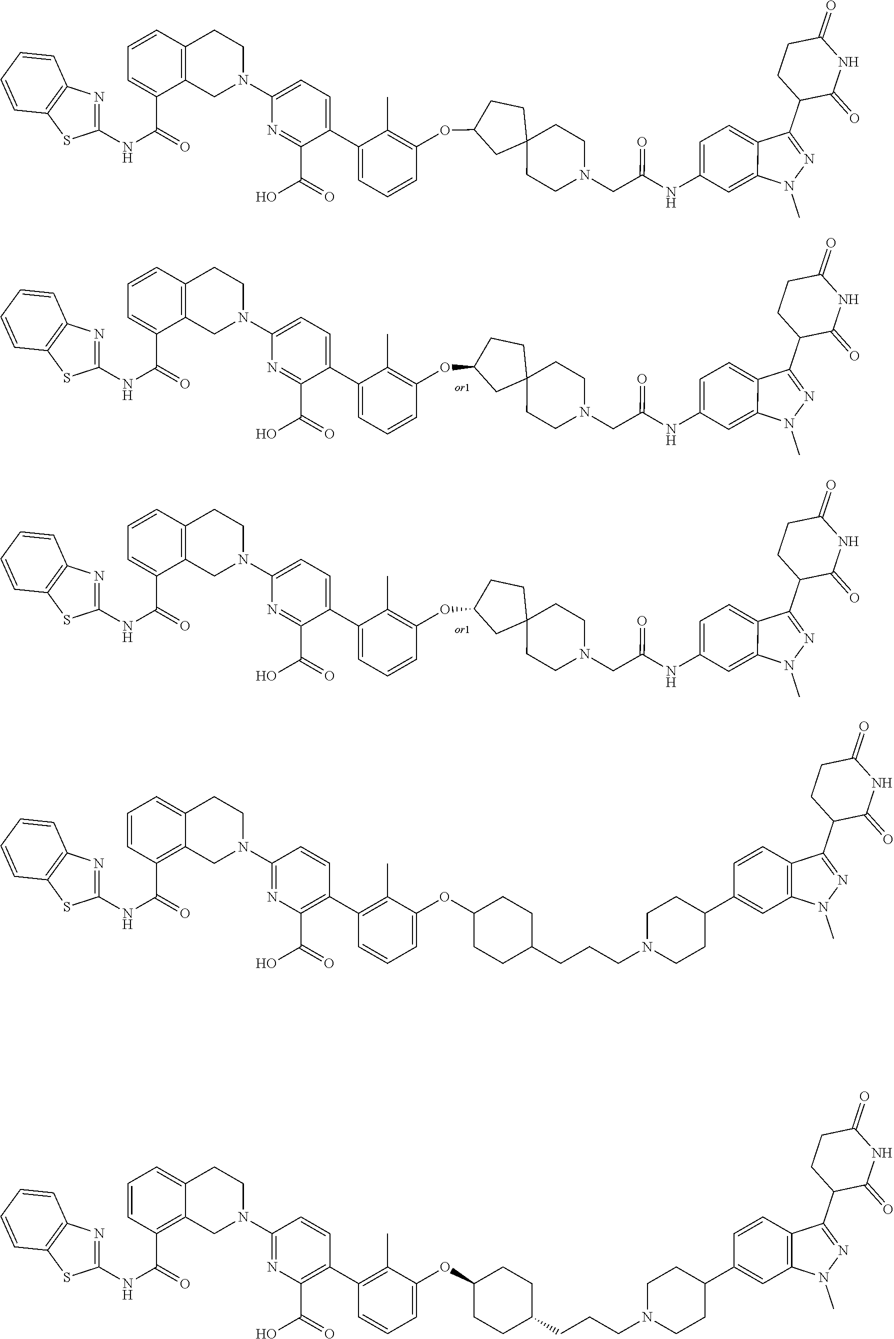 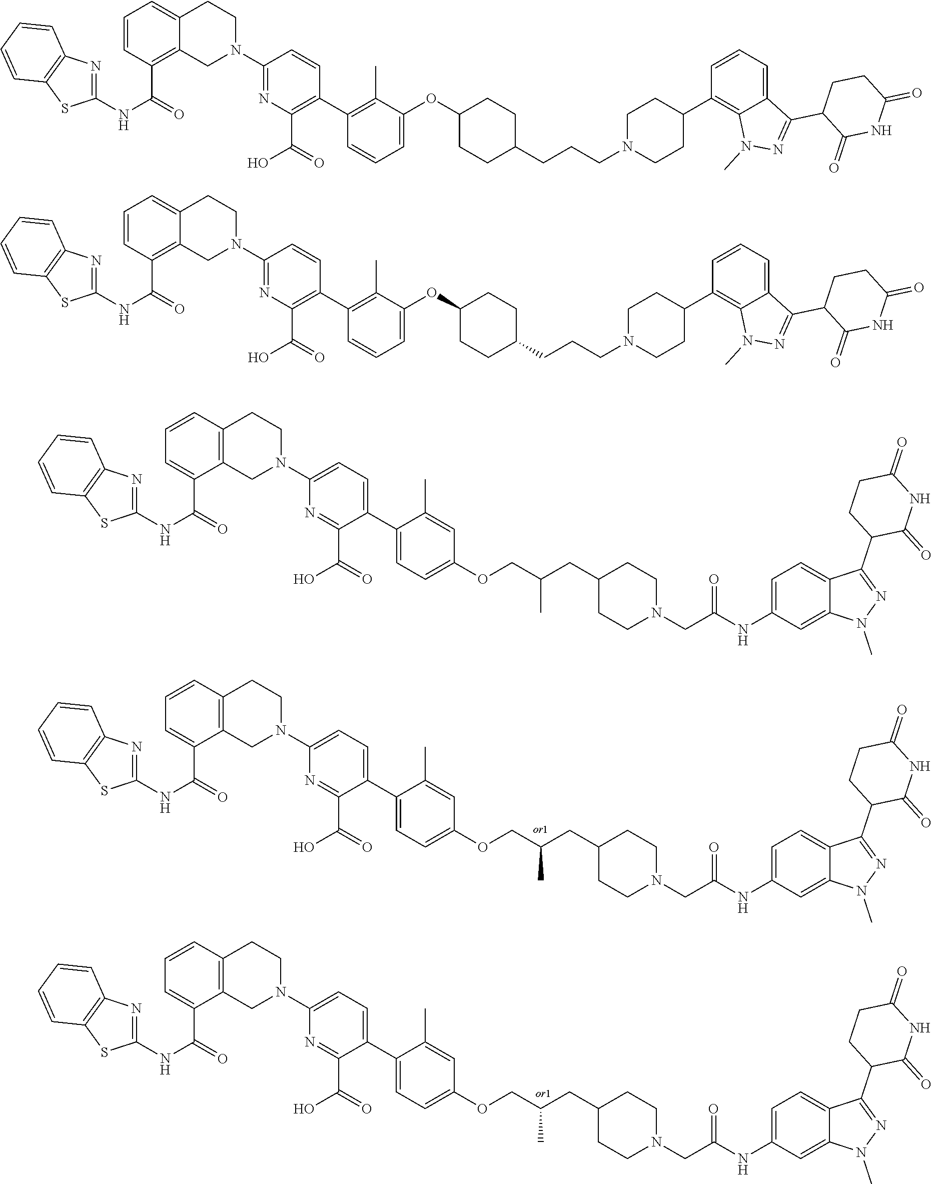 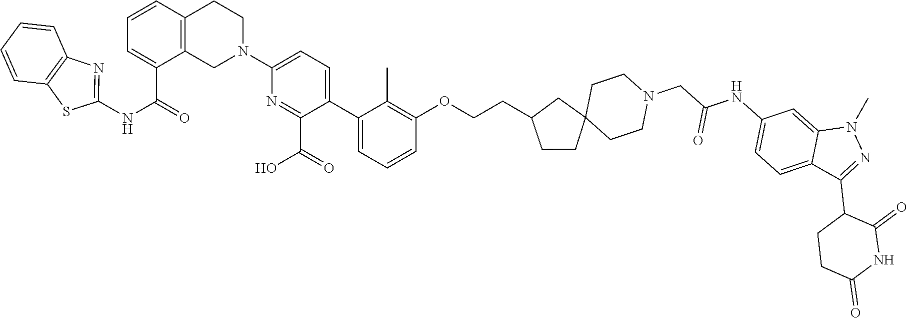 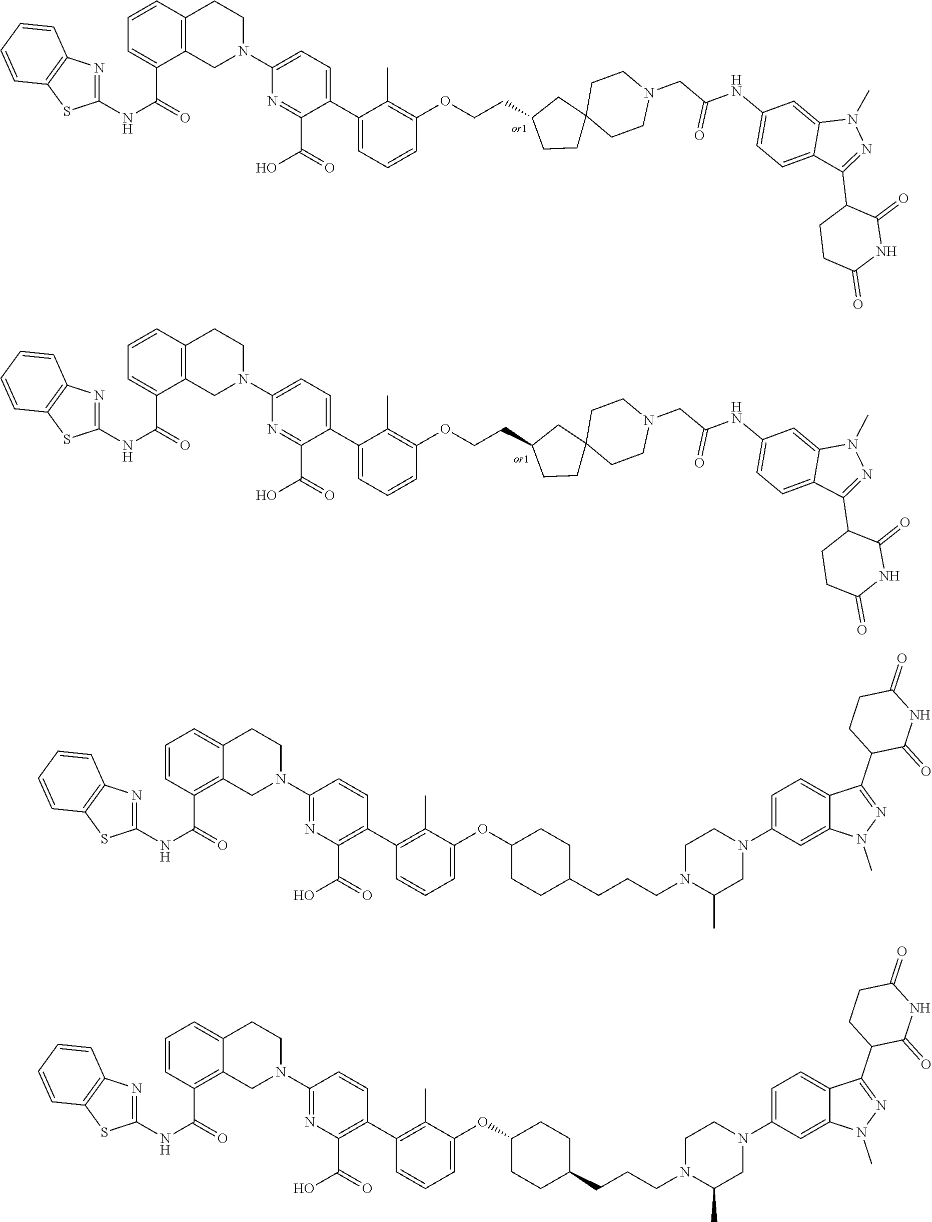 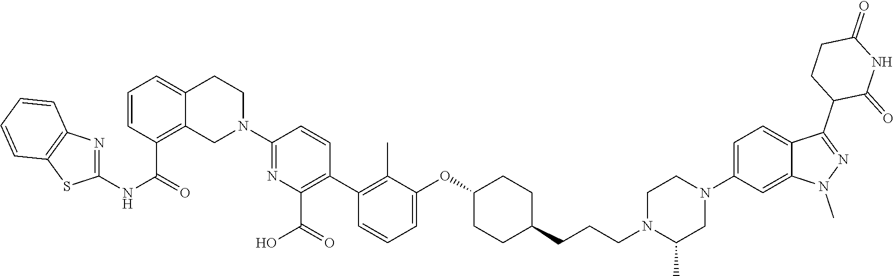 [SMILES: Cc1c(O[C@H]2CC[C@H](CCCN3CCN(c4ccc5c(C6CCC(=O)NC6=O)nn(C)c5c4)C[C@@H]3C)CC2)cccc1-c1ccc(N2CCc3cccc(C(=O)Nc4nc5ccccc5s4)c3C2)nc1C(=O)O] 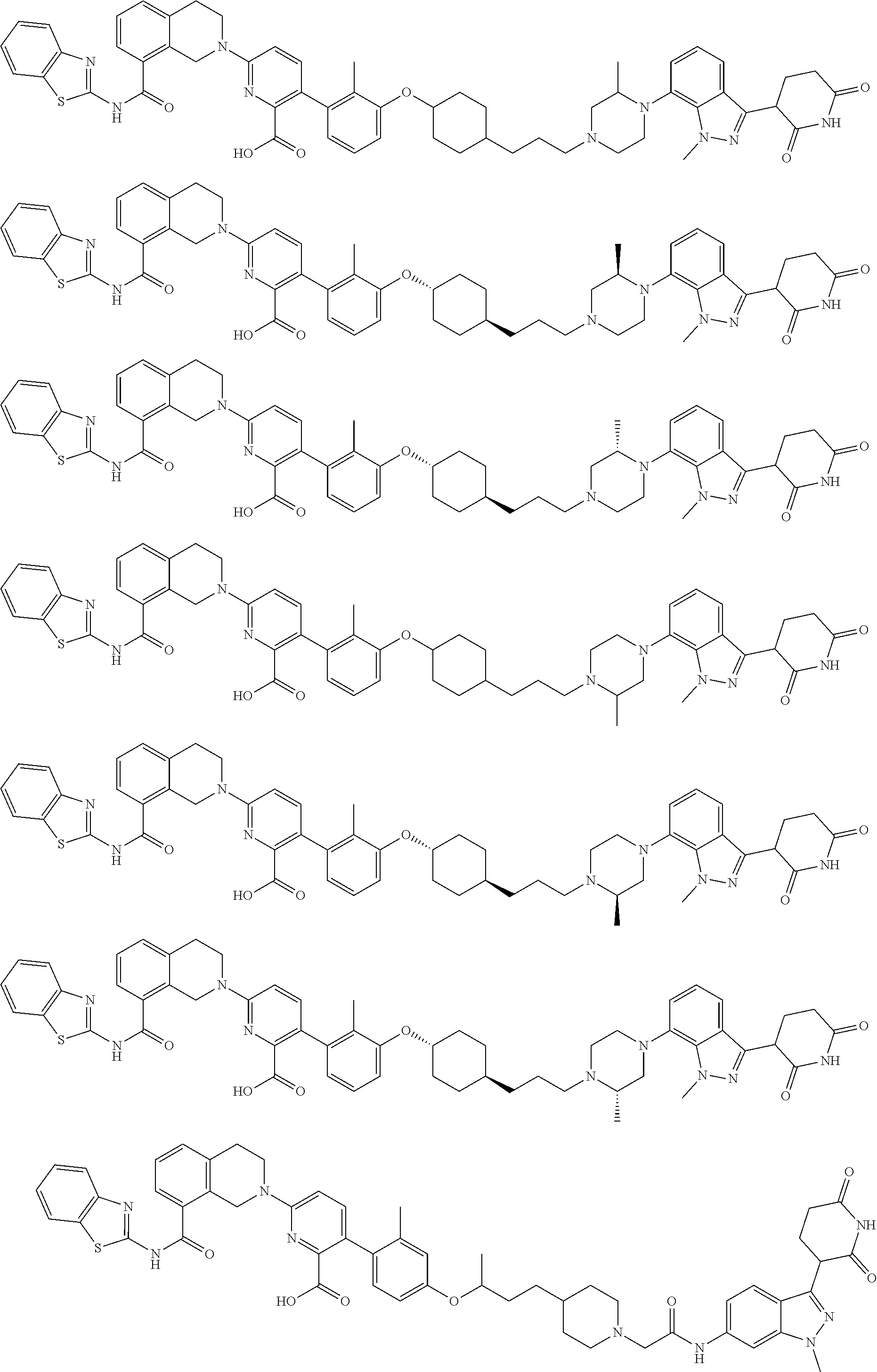 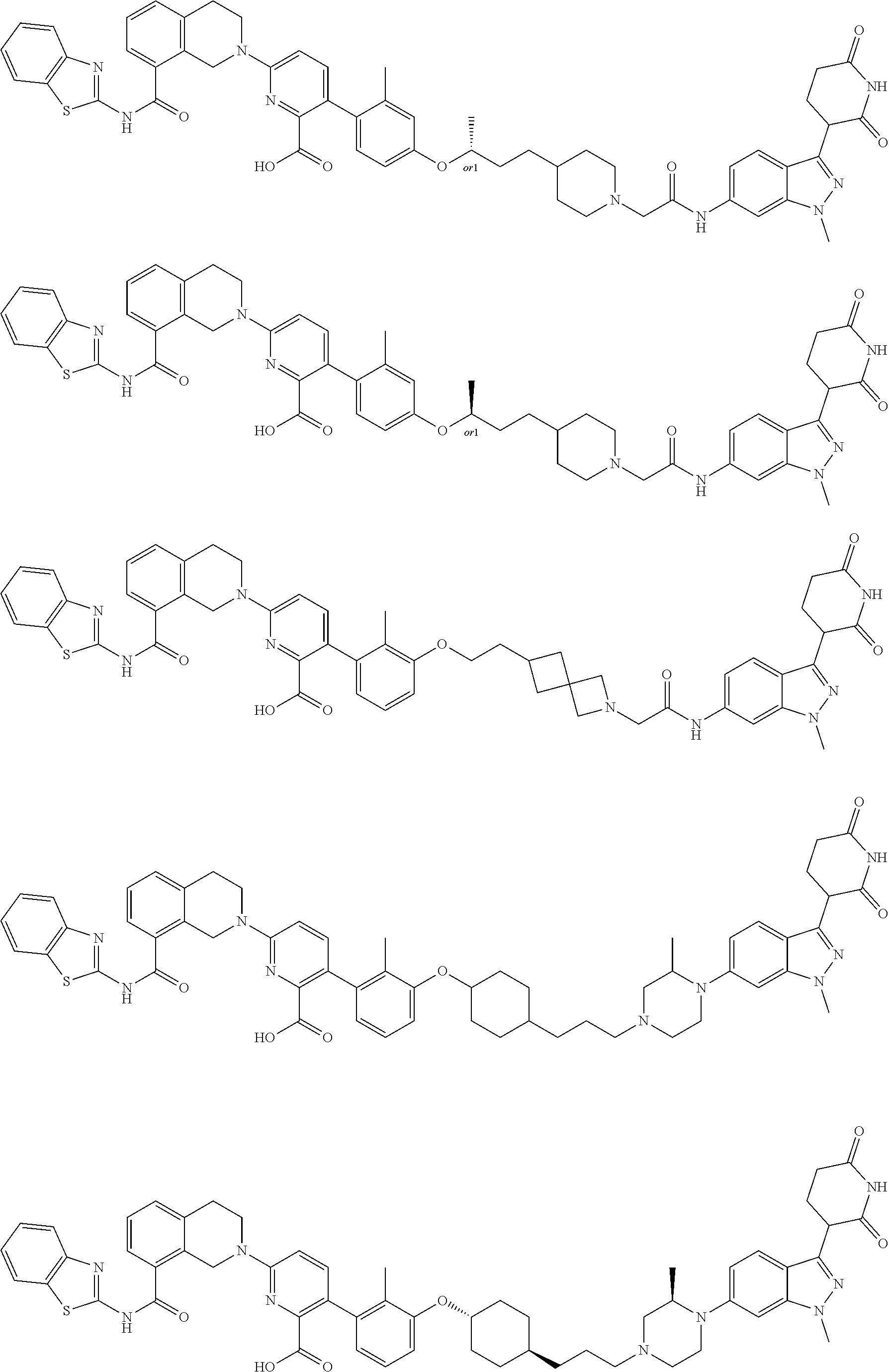 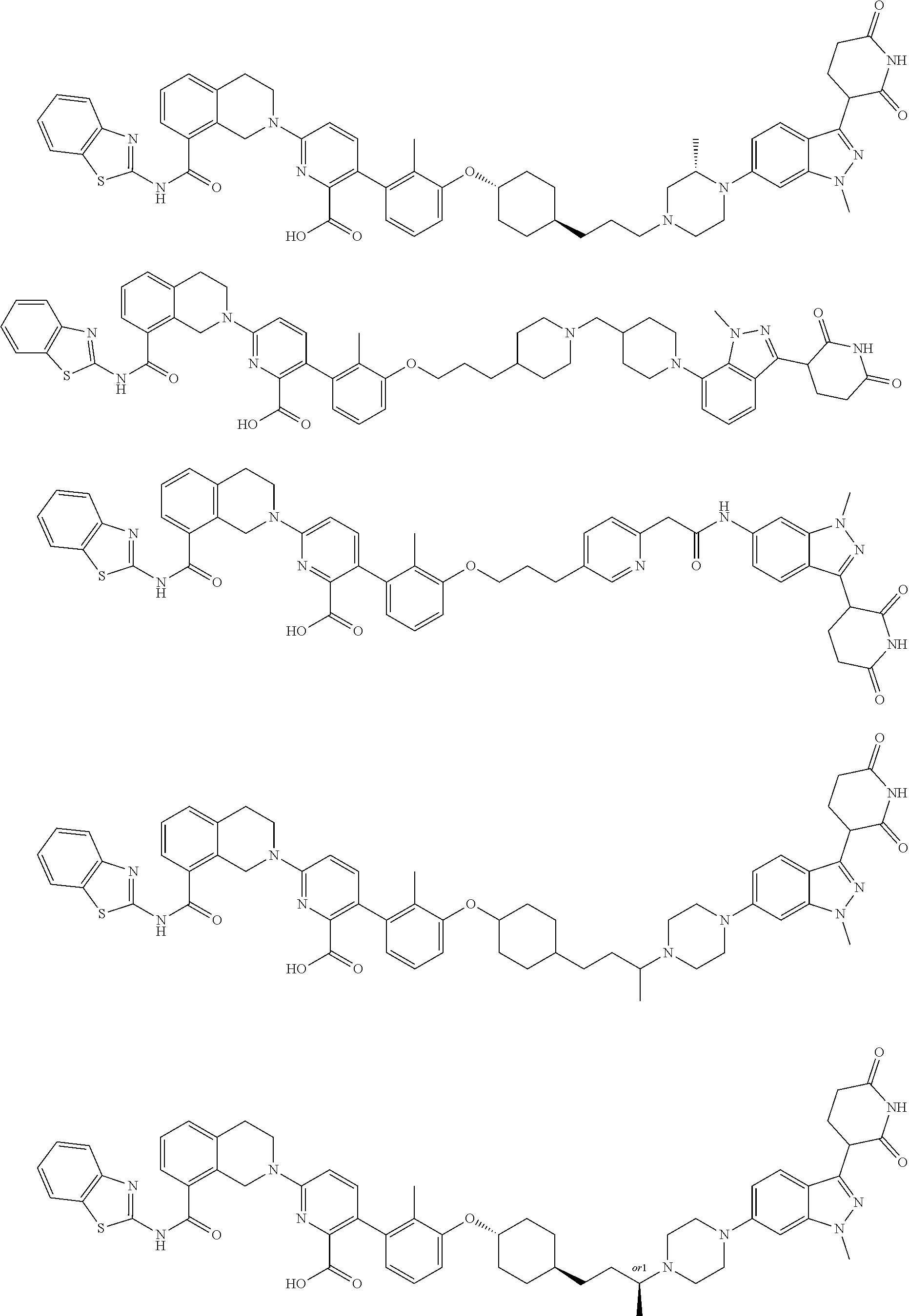 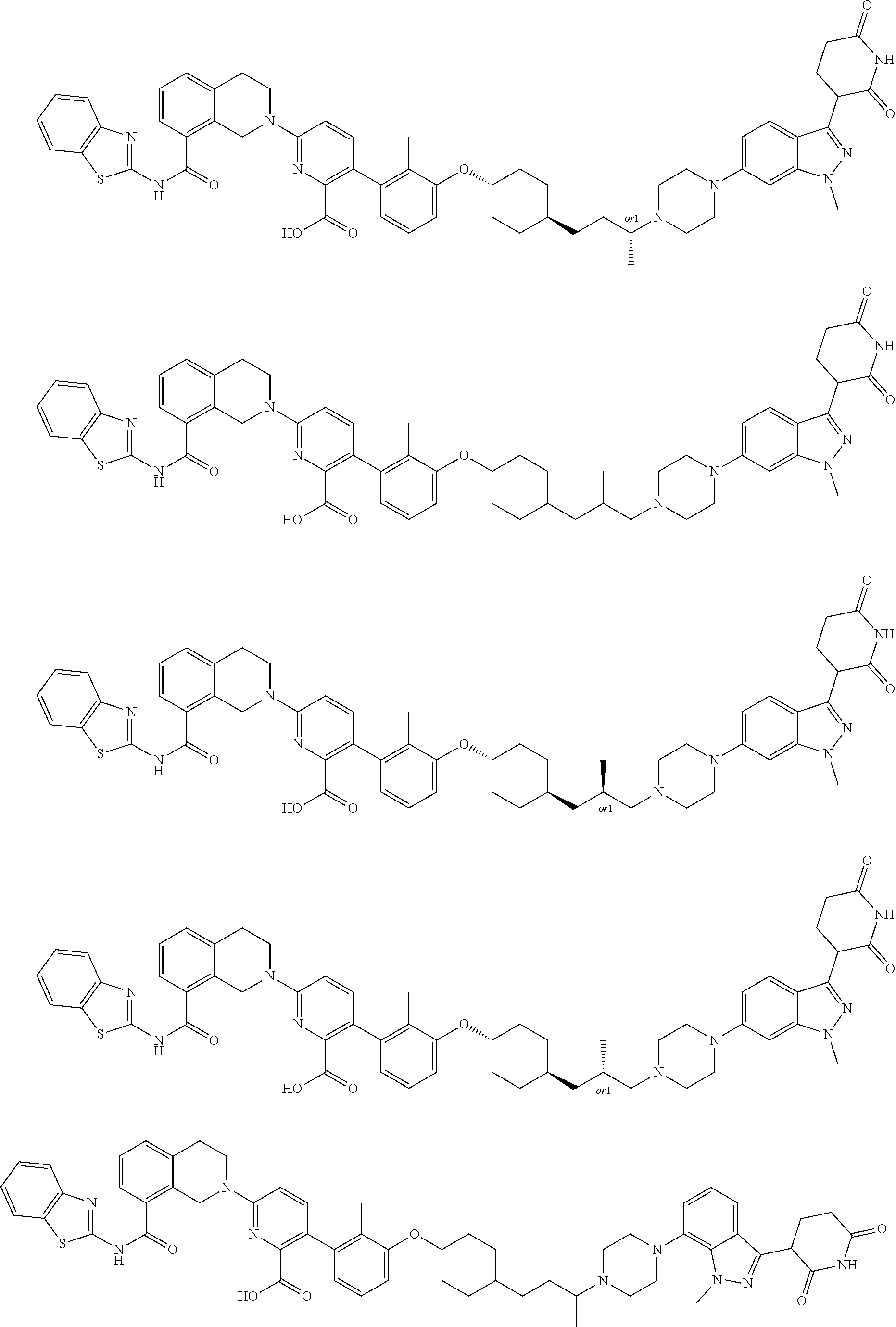 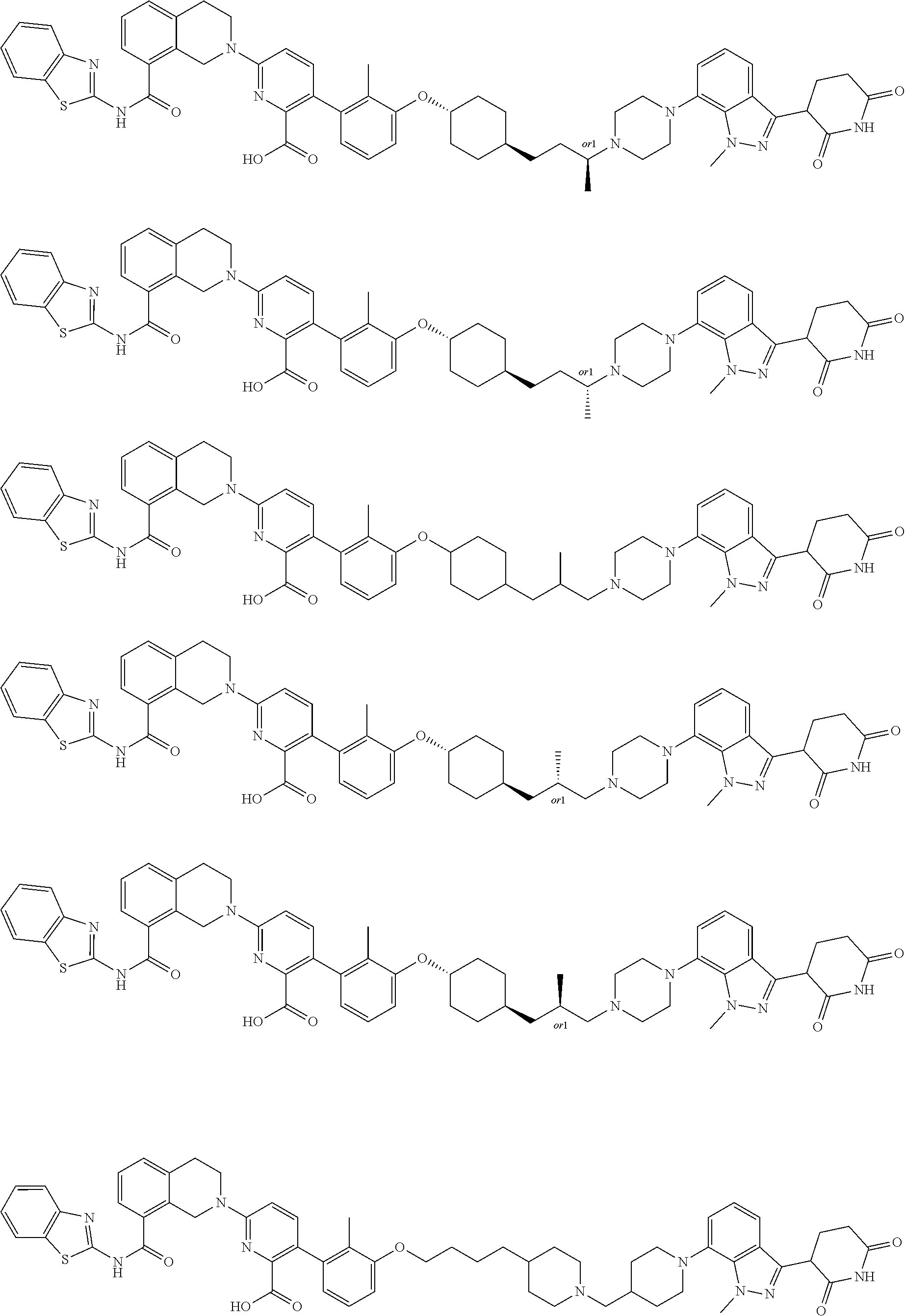 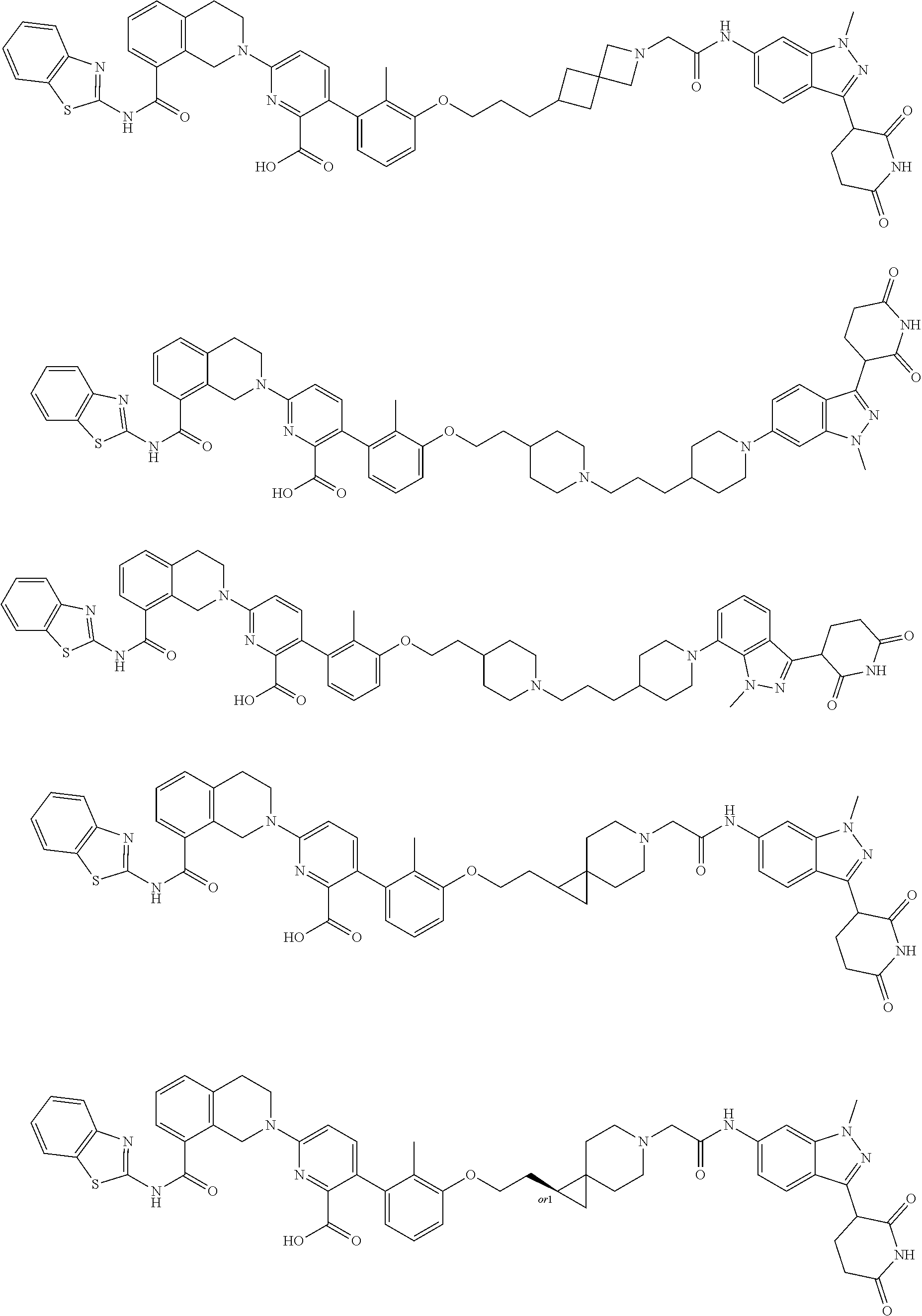 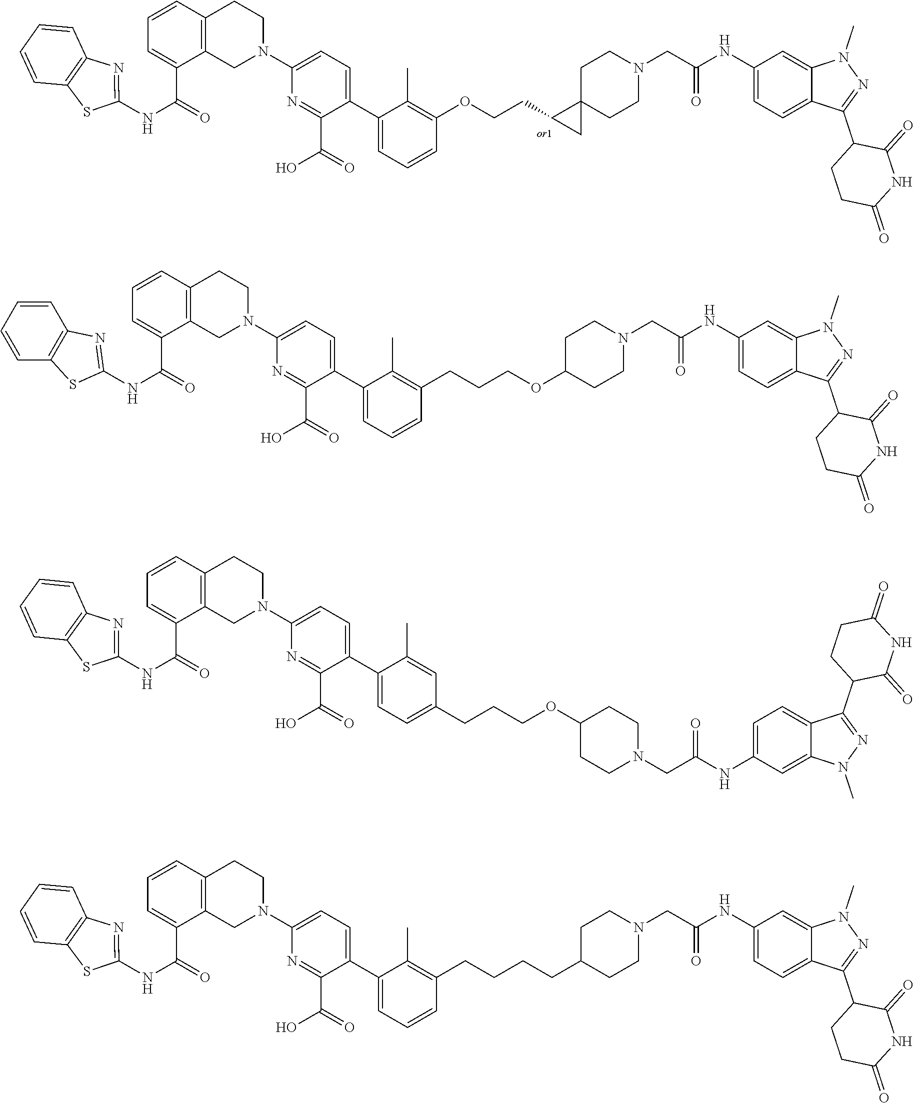 [SMILES: Cc1c(OCC[C@H]2CC23CCN(CC(=O)Nc2ccc4c(C5CCC(=O)NC5=O)nn(C)c4c2)CC3)cccc1-c1ccc(N2CCc3cccc(C(=O)Nc4nc5ccccc5s4)c3C2)nc1C(=O)O ; Cc1c(CCCOC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O ; Cc1cc(CCCOC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O ; Cc1c(CCCCC2CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O] 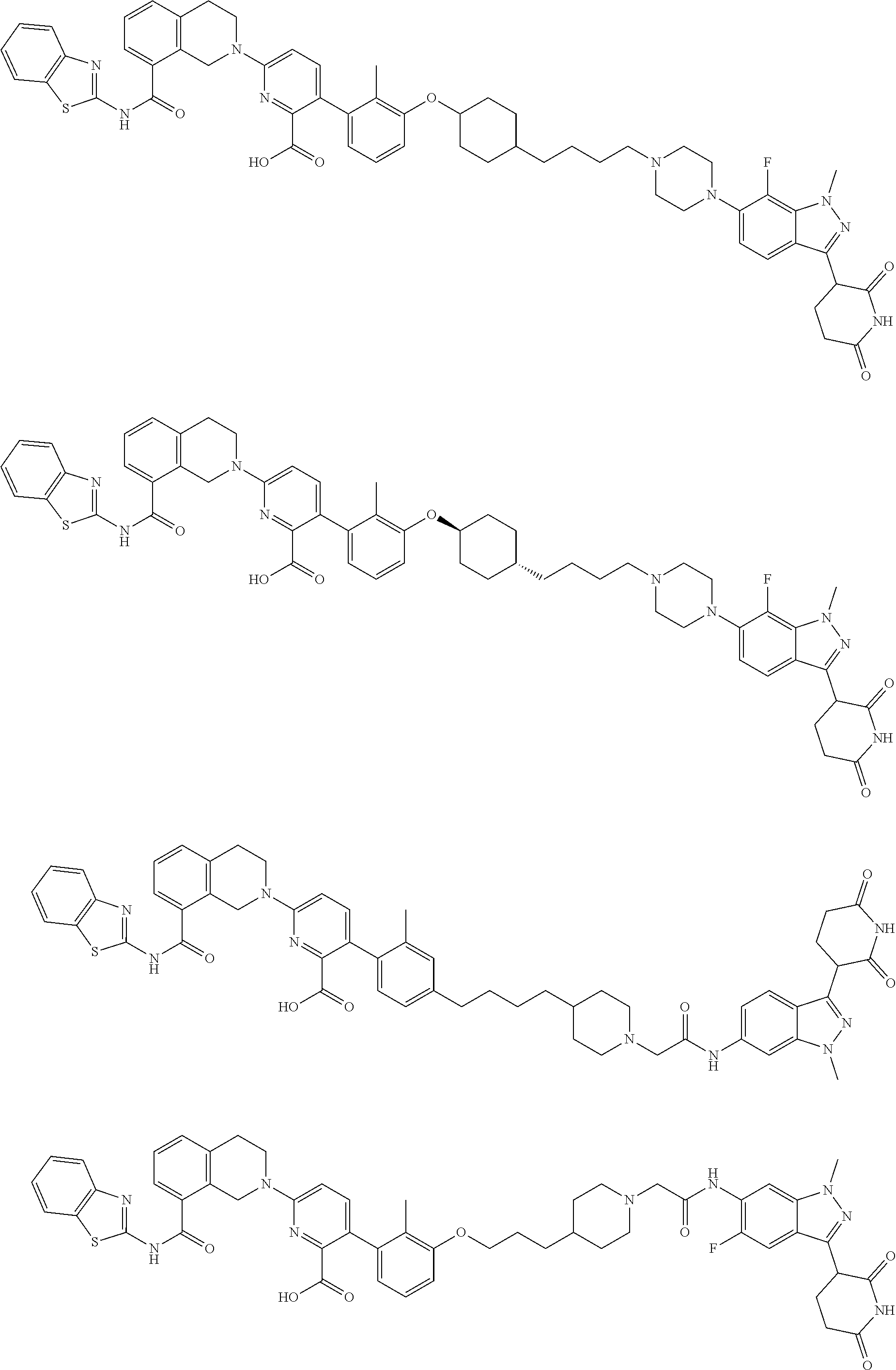 [SMILES: Cc1c(OC2CCC(CCCCN3CCN(c4ccc5c(C6CCC(=O)NC6=O)nn(C)c5c4F)CC3)CC2)cccc1-c1ccc(N2CCc3cccc(C(=O)Nc4nc5ccccc5s4)c3C2)nc1C(=O)O ; Cc1c(O[C@H]2CC[C@H](CCCCN3CCN(c4ccc5c(C6CCC(=O)NC6=O)nn(C)c5c4F)CC3)CC2)cccc1-c1ccc(N2CCc3cccc(C(=O)Nc4nc5ccccc5s4)c3C2)nc1C(=O)O ; Cc1cc(CCCCC2CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O ; Cc1c(OCCCC2CCN(CC(=O)Nc3cc4c(cc3F)c(C3CCC(=O)NC3=O)nn4C)CC2)cccc1-c1ccc(N2CCc3cccc(C(=O)Nc4nc5ccccc5s4)c3C2)nc1C(=O)O] 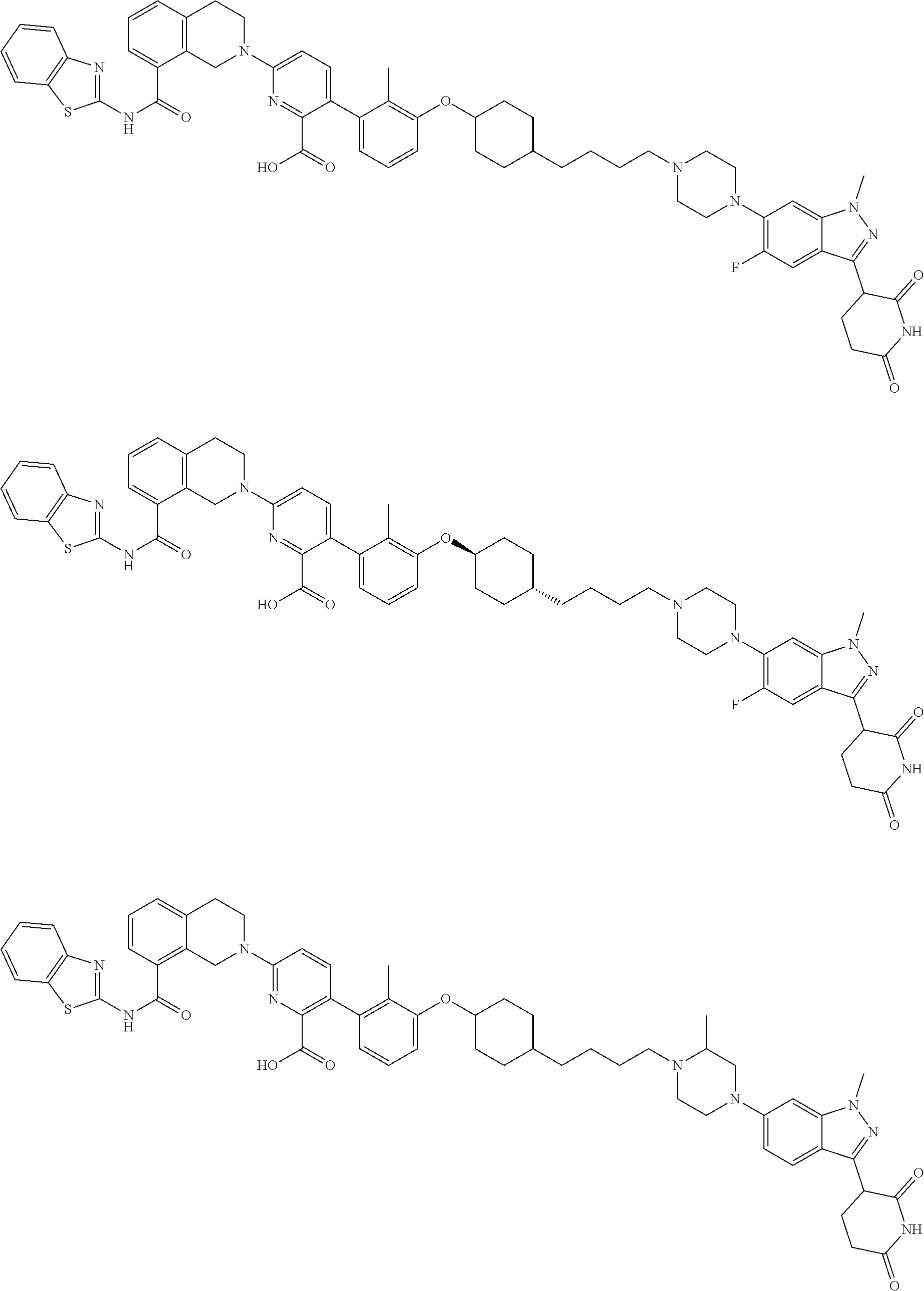 [SMILES: Cc1c(OC2CCC(CCCCN3CCN(c4cc5c(cc4F)c(C4CCC(=O)NC4=O)nn5C)CC3)CC2)cccc1-c1ccc(N2CCc3cccc(C(=O)Nc4nc5ccccc5s4)c3C2)nc1C(=O)O ; Cc1c(O[C@H]2CC[C@H](CCCCN3CCN(c4cc5c(cc4F)c(C4CCC(=O)NC4=O)nn5C)CC3)CC2)cccc1-c1ccc(N2CCc3cccc(C(=O)Nc4nc5ccccc5s4)c3C2)nc1C(=O)O ; Cc1c(OC2CCC(CCCCN3CCN(c4ccc5c(C6CCC(=O)NC6=O)nn(C)c5c4)CC3C)CC2)cccc1-c1ccc(N2CCc3cccc(C(=O)Nc4nc5ccccc5s4)c3C2)nc1C(=O)O] 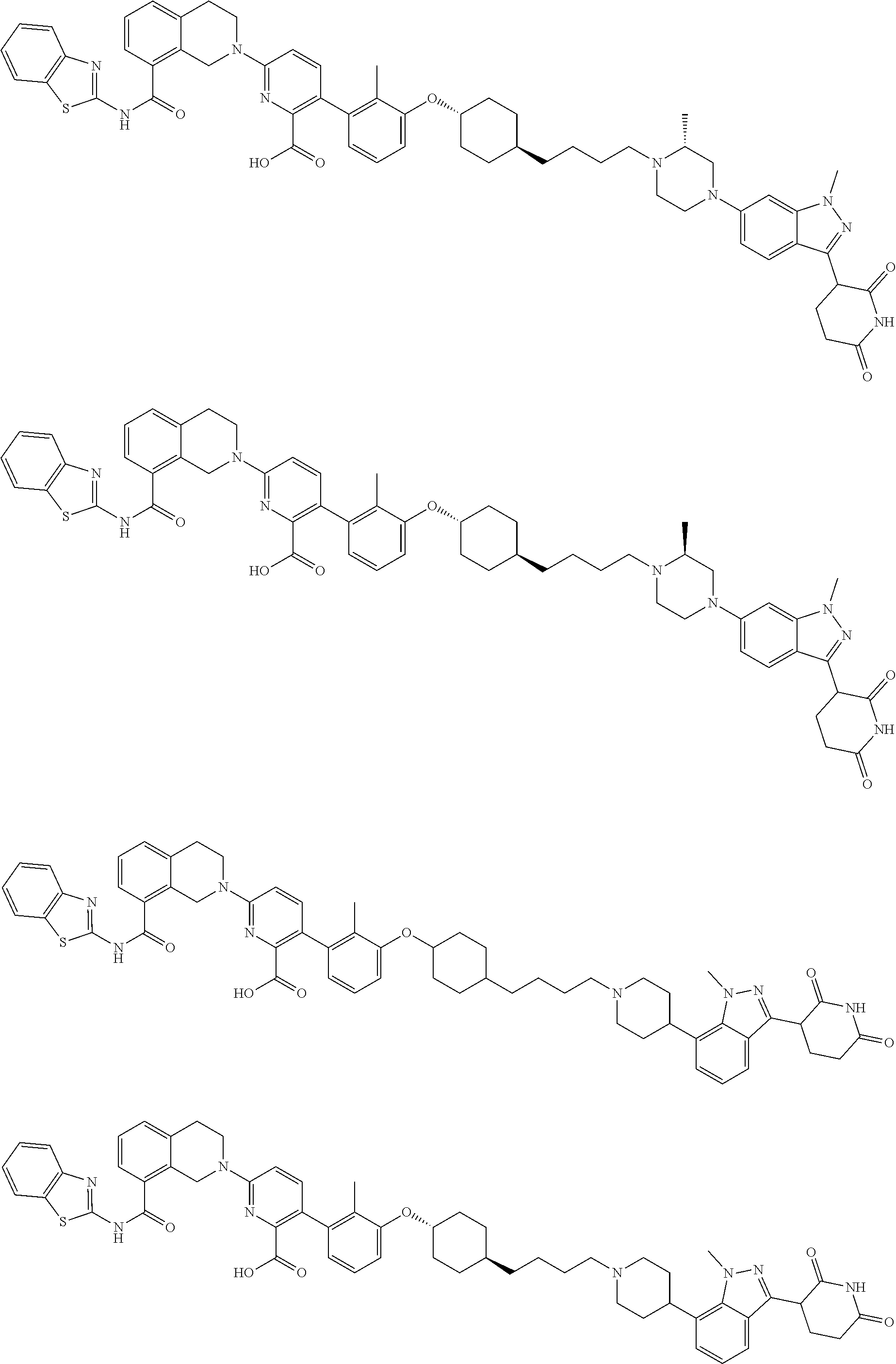 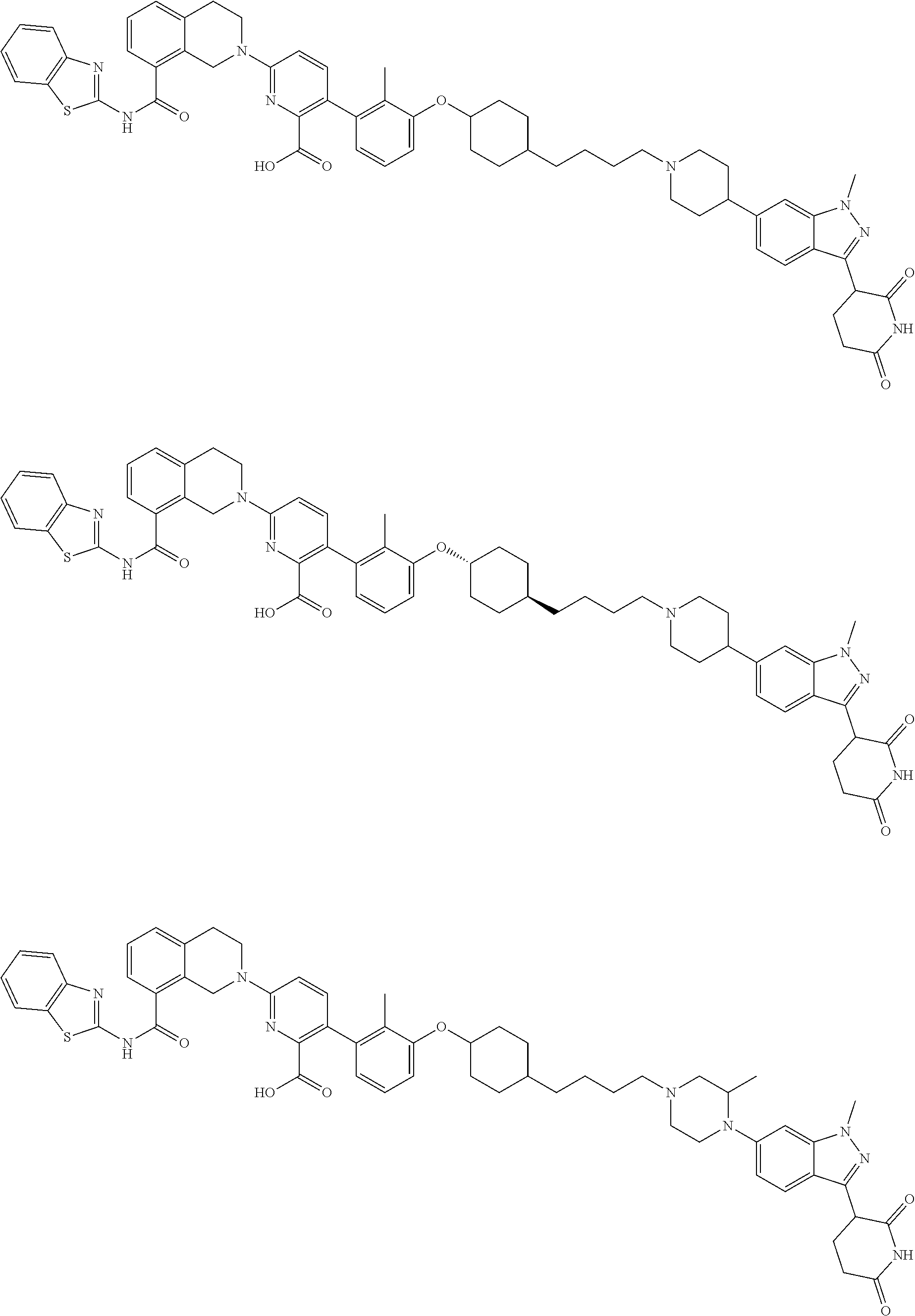 [SMILES: Cc1c(OC2CCC(CCCCN3CCC(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O ; Cc1c(O[C@H]2CC[C@H](CCCCN3CCC(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O ; Cc1c(OC2CCC(CCCCN3CCN(c4ccc5c(C6CCC(=O)NC6=O)nn(C)c5c4)C(C)C3)CC2)cccc1-c1ccc(N2CCc3cccc(C(=O)Nc4nc5ccccc5s4)c3C2)nc1C(=O)O] 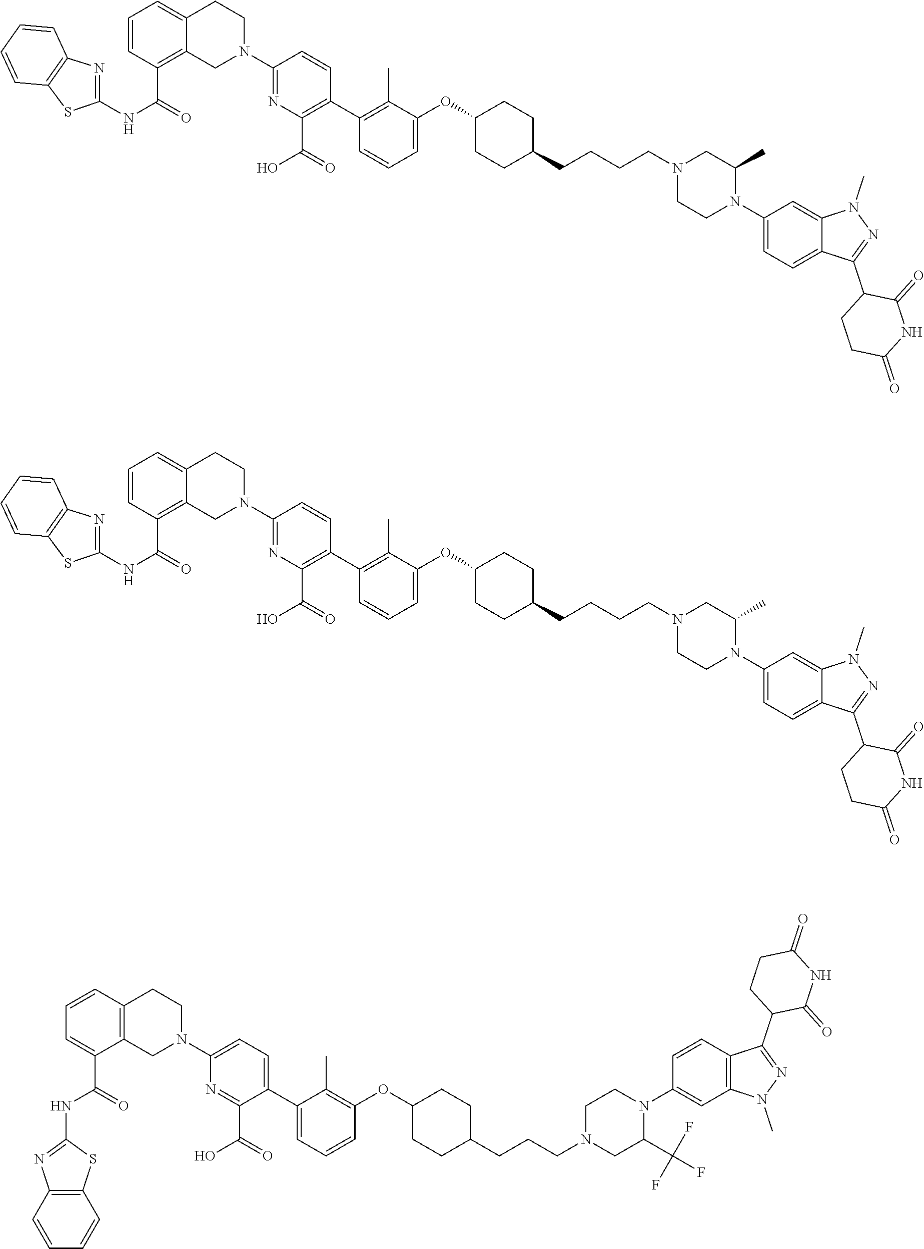 [SMILES: Cc1c(O[C@H]2CC[C@H](CCCCN3CCN(c4ccc5c(C6CCC(=O)NC6=O)nn(C)c5c4)[C@H](C)C3)CC2)cccc1-c1ccc(N2CCc3cccc(C(=O)Nc4nc5ccccc5s4)c3C2)nc1C(=O)O ; Cc1c(O[C@H]2CC[C@H](CCCCN3CCN(c4ccc5c(C6CCC(=O)NC6=O)nn(C)c5c4)[C@@H](C)C3)CC2)cccc1-c1ccc(N2CCc3cccc(C(=O)Nc4nc5ccccc5s4)c3C2)nc1C(=O)O ; Cc1c(OC2CCC(CCCN3CCN(c4ccc5c(C6CCC(=O)NC6=O)nn(C)c5c4)C(C(F)(F)F)C3)CC2)cccc1-c1ccc(N2CCc3cccc(C(=O)Nc4nc5ccccc5s4)c3C2)nc1C(=O)O] 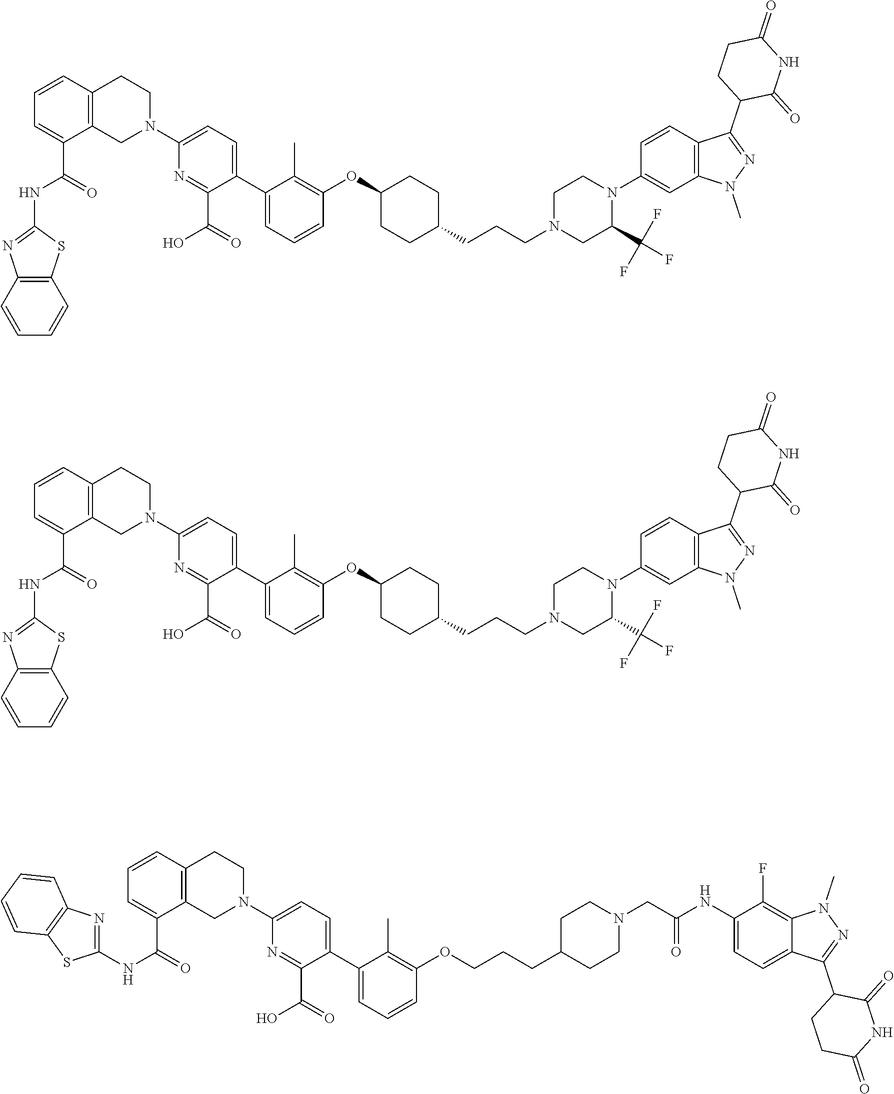 [SMILES: Cc1c(O[C@H]2CC[C@H](CCCN3CCN(c4ccc5c(C6CCC(=O)NC6=O)nn(C)c5c4)[C@@H](C(F)(F)F)C3)CC2)cccc1-c1ccc(N2CCc3cccc(C(=O)Nc4nc5ccccc5s4)c3C2)nc1C(=O)O ; Cc1c(O[C@H]2CC[C@H](CCCN3CCN(c4ccc5c(C6CCC(=O)NC6=O)nn(C)c5c4)[C@H](C(F)(F)F)C3)CC2)cccc1-c1ccc(N2CCc3cccc(C(=O)Nc4nc5ccccc5s4)c3C2)nc1C(=O)O ; Cc1c(OCCCC2CCN(CC(=O)Nc3ccc4c(C5CCC(=O)NC5=O)nn(C)c4c3F)CC2)cccc1-c1ccc(N2CCc3cccc(C(=O)Nc4nc5ccccc5s4)c3C2)nc1C(=O)O] 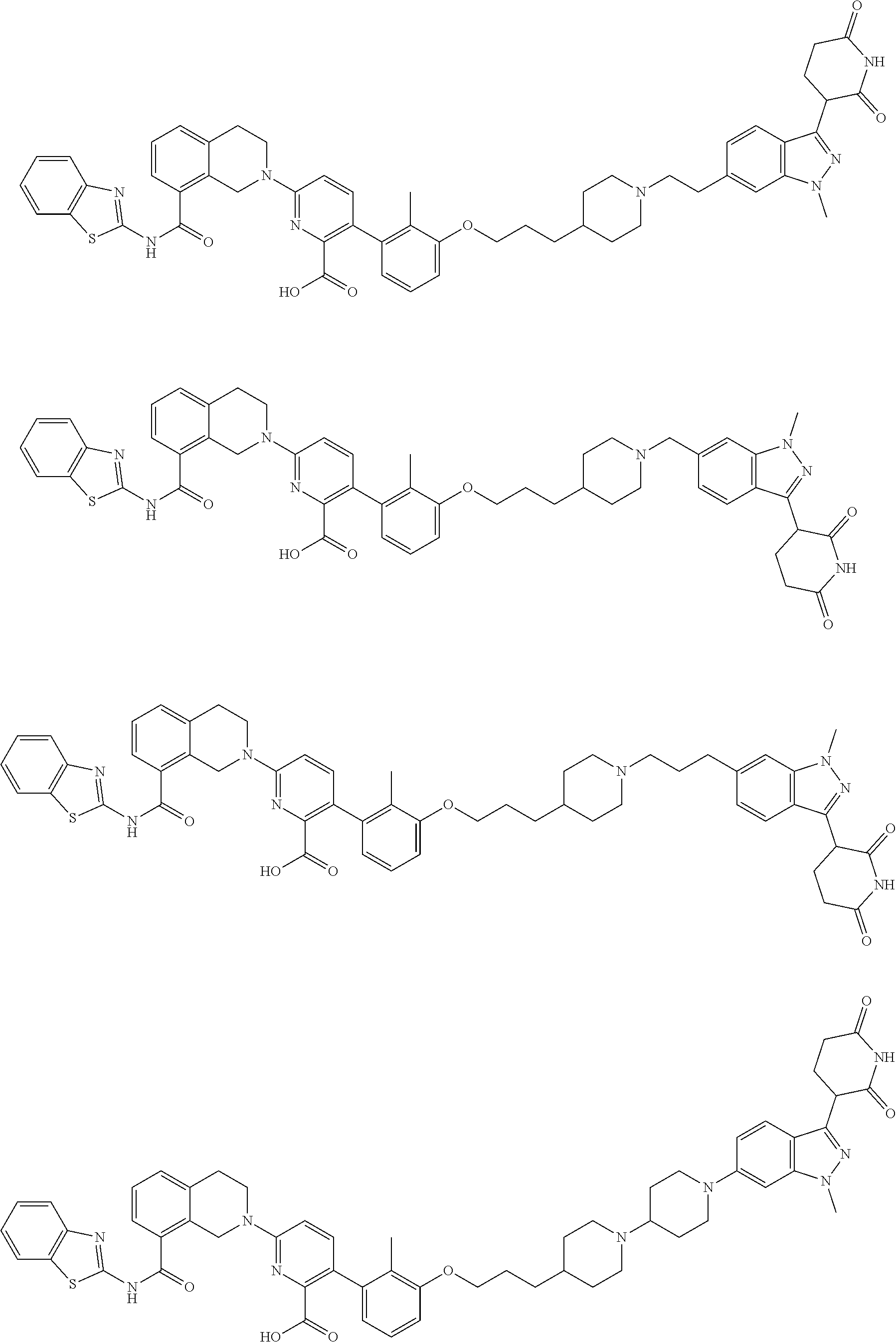 [SMILES: Cc1c(OCCCC2CCN(CCc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O ; Cc1c(OCCCC2CCN(Cc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O ; Cc1c(OCCCC2CCN(CCCc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O ; Cc1c(OCCCC2CCN(C3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] 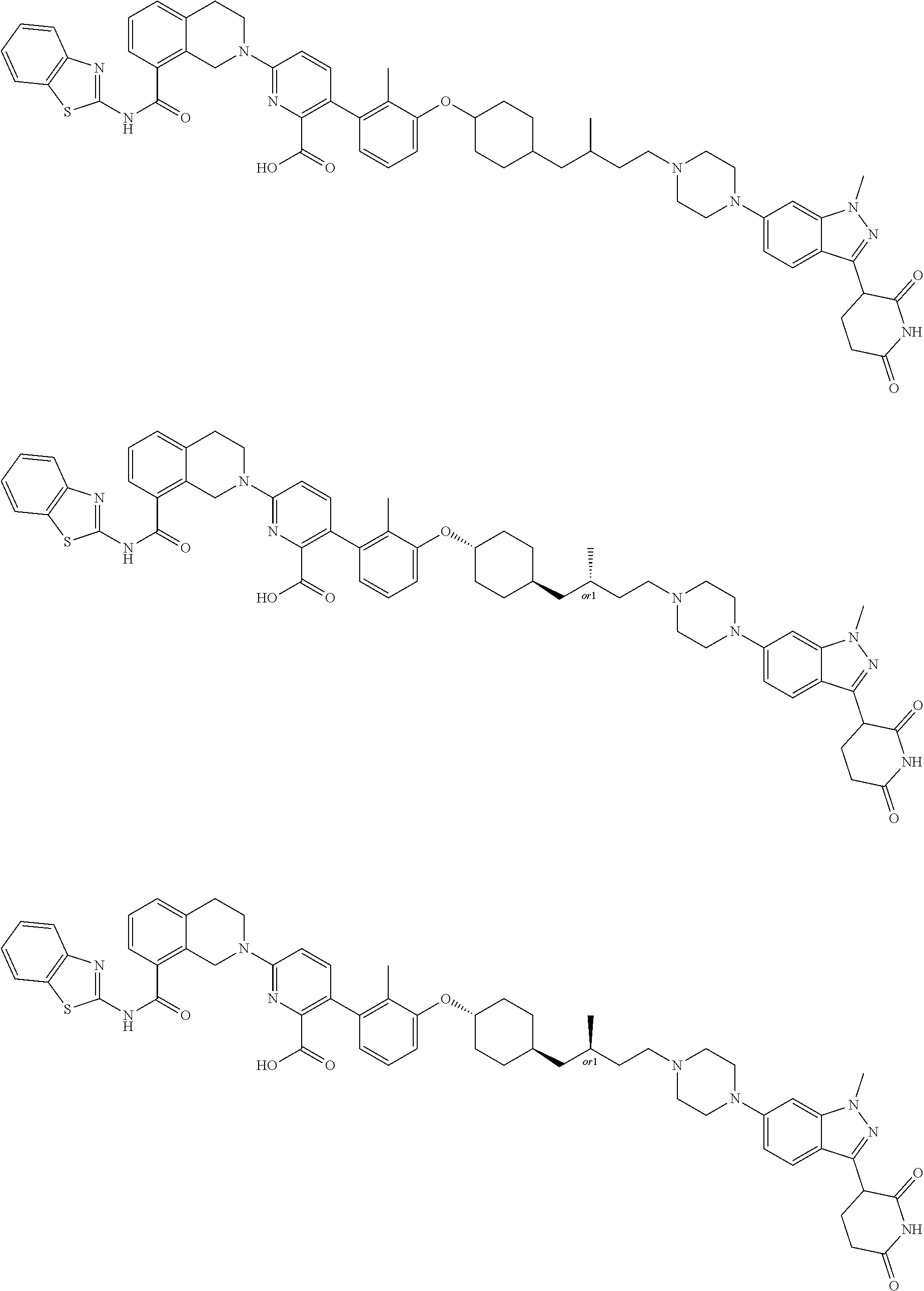 [SMILES: Cc1c(OC2CCC(CC(C)CCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O ; Cc1c(O[C@H]2CC[C@H](C[C@H](C)CCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O ; Cc1c(O[C@H]2CC[C@H](C[C@@H](C)CCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] 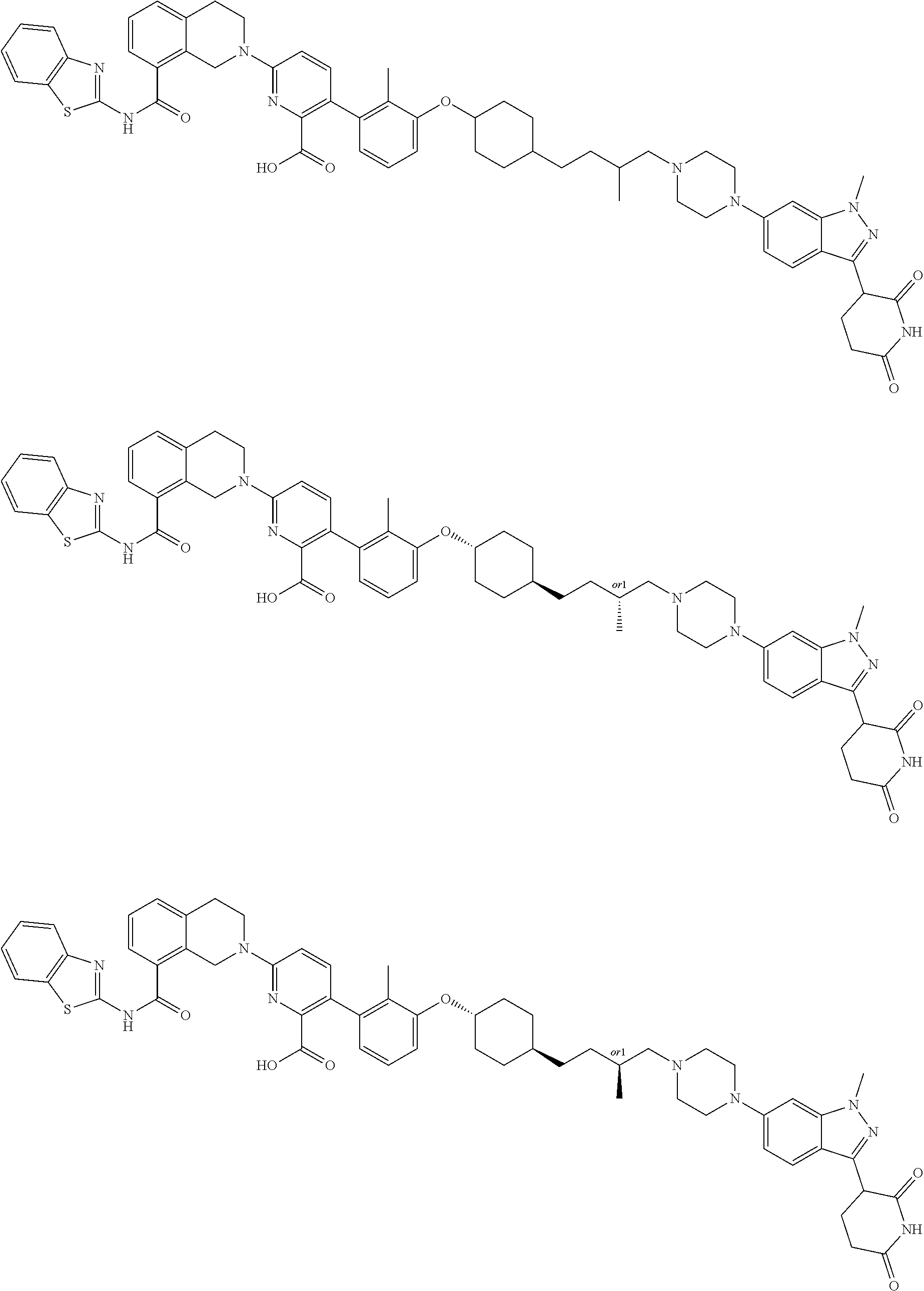 [SMILES: Cc1c(OC2CCC(CCC(C)CN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O ; Cc1c(O[C@H]2CC[C@H](CC[C@@H](C)CN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O ; Cc1c(O[C@H]2CC[C@H](CC[C@H](C)CN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] 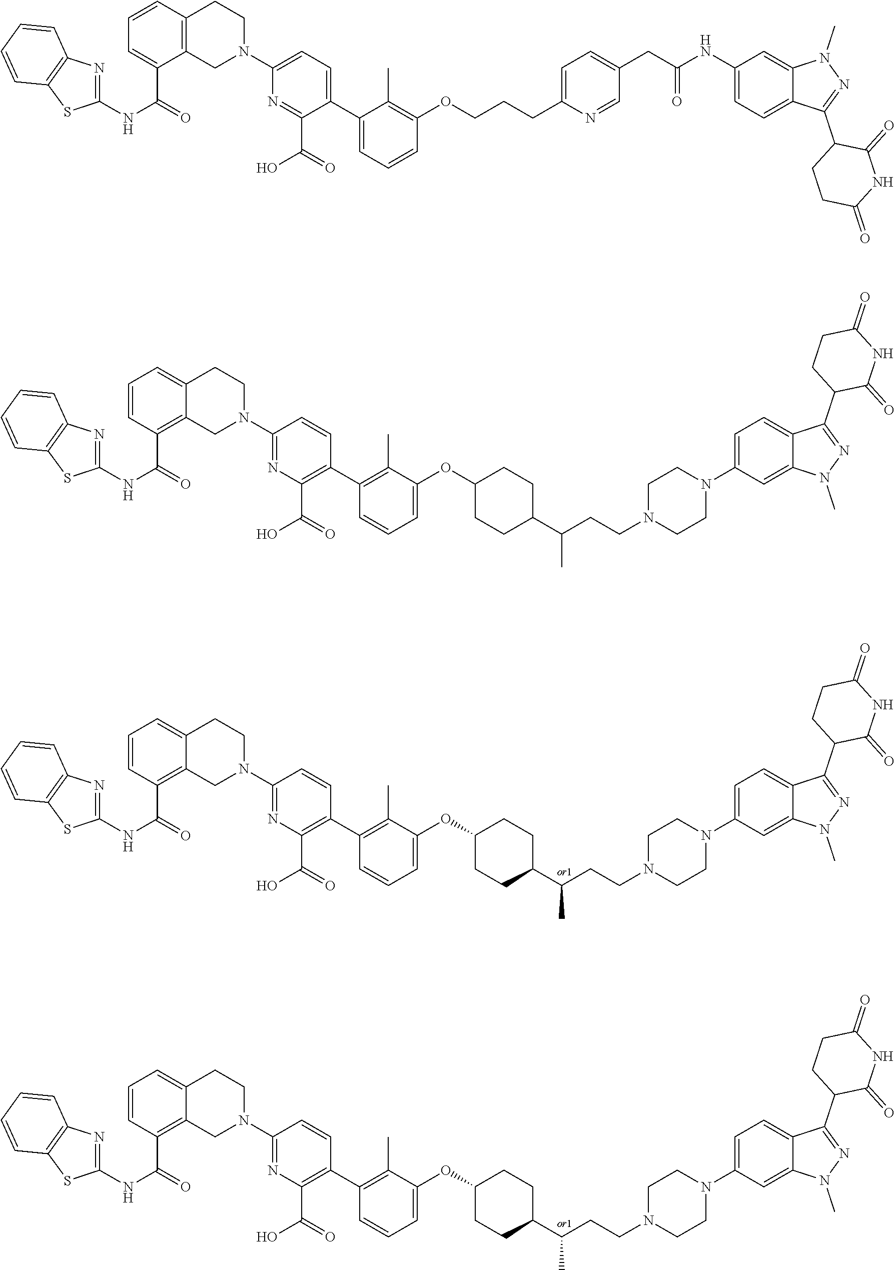 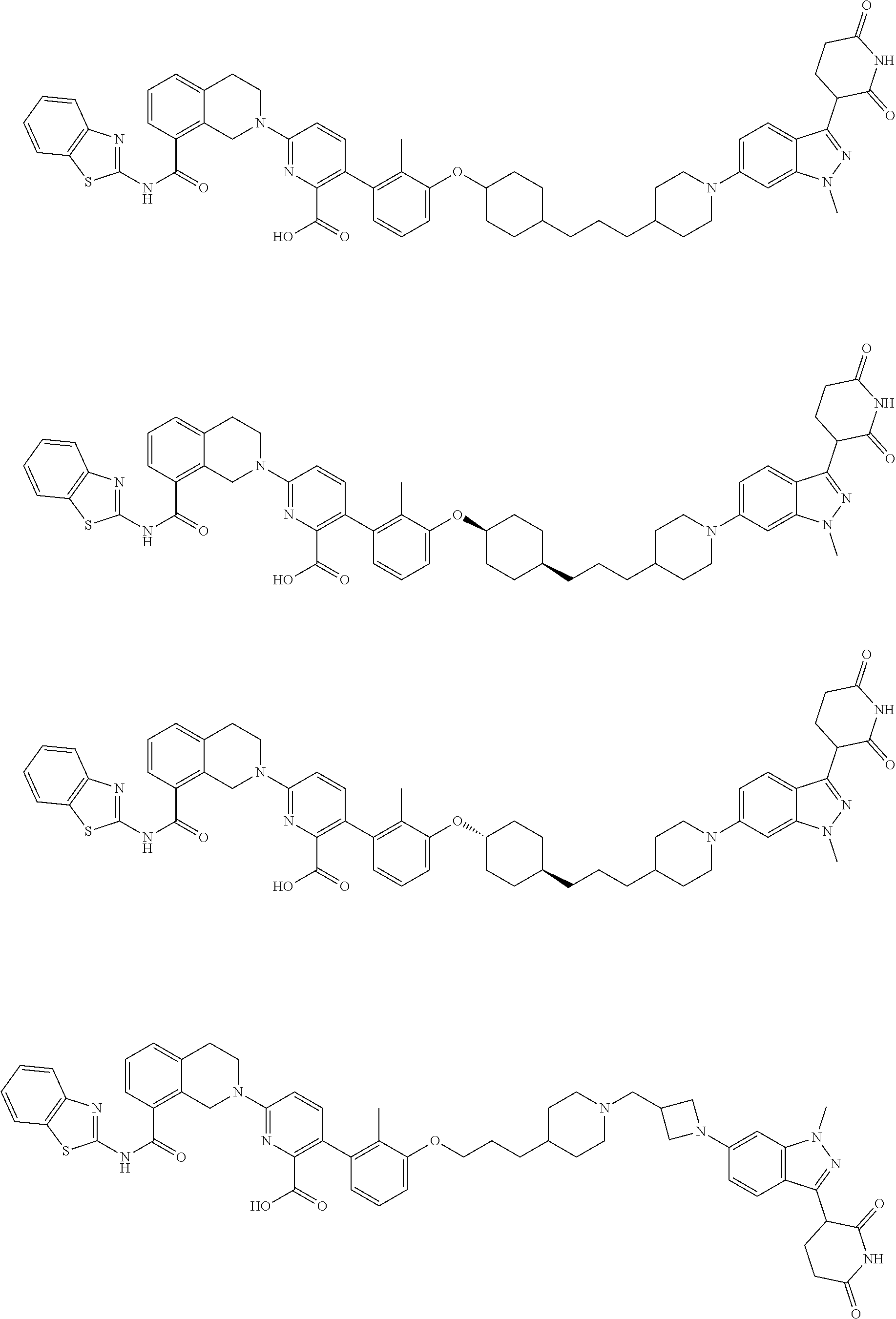 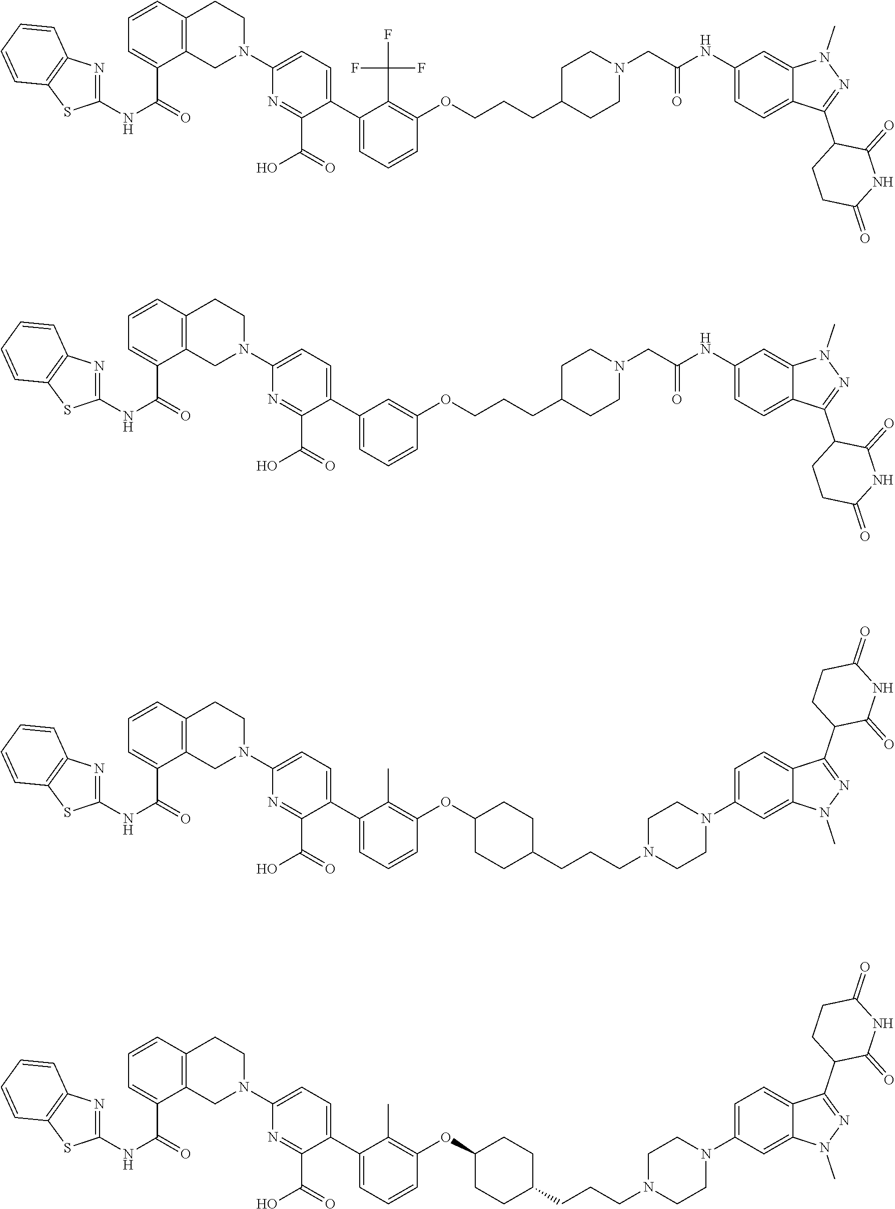 [SMILES: Cn1nc(C2CCC(=O)NC2=O)c2ccc(NC(=O)CN3CCC(CCCOc4cccc(-c5ccc(N6CCc7cccc(C(=O)Nc8nc9ccccc9s8)c7C6)nc5C(=O)O)c4C(F)(F)F)CC3)cc21 ; Cn1nc(C2CCC(=O)NC2=O)c2ccc(NC(=O)CN3CCC(CCCOc4cccc(-c5ccc(N6CCc7cccc(C(=O)Nc8nc9ccccc9s8)c7C6)nc5C(=O)O)c4)CC3)cc21 ; Cc1c(OC2CCC(CCCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O ; Cc1c(O[C@H]2CC[C@H](CCCN3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] 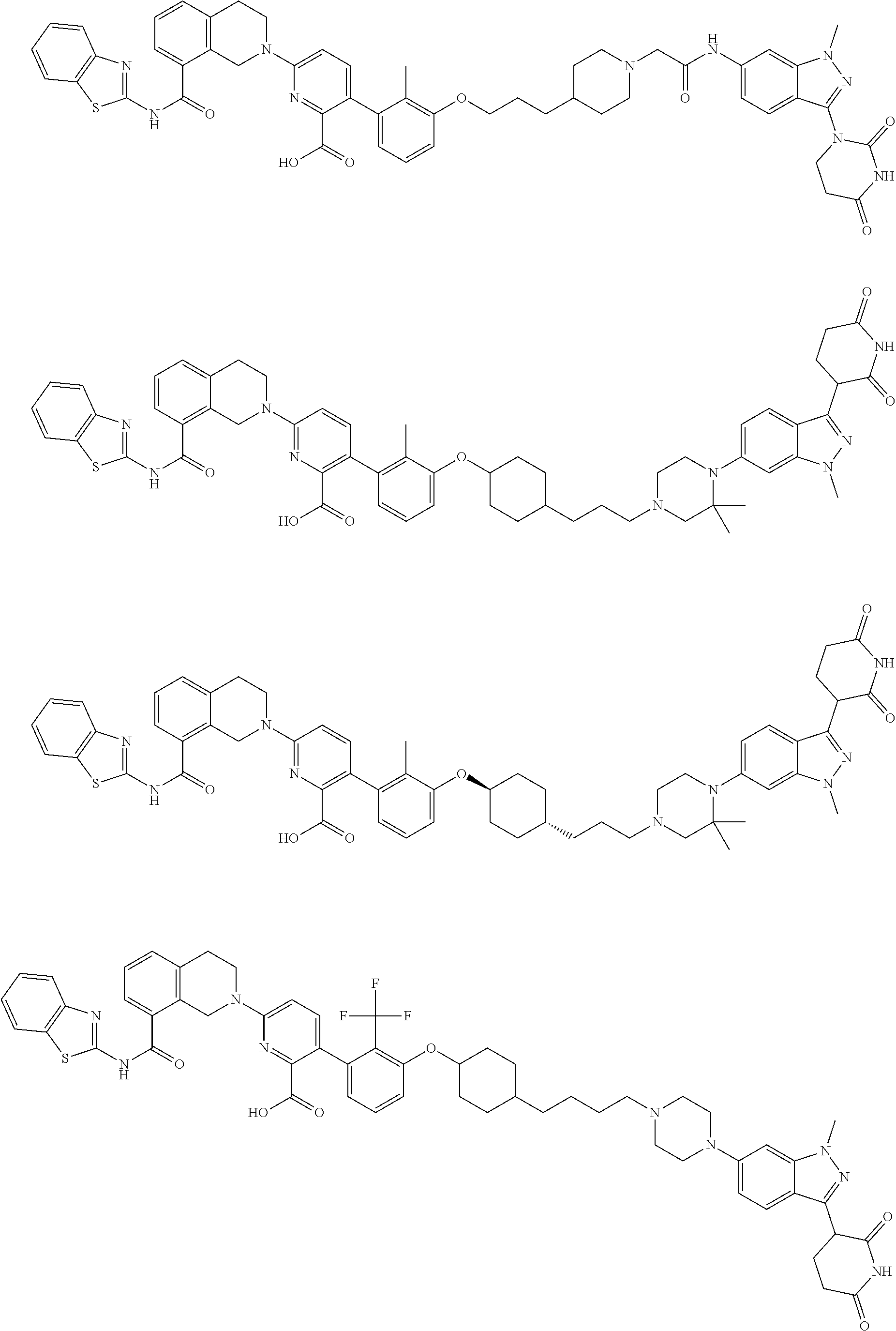 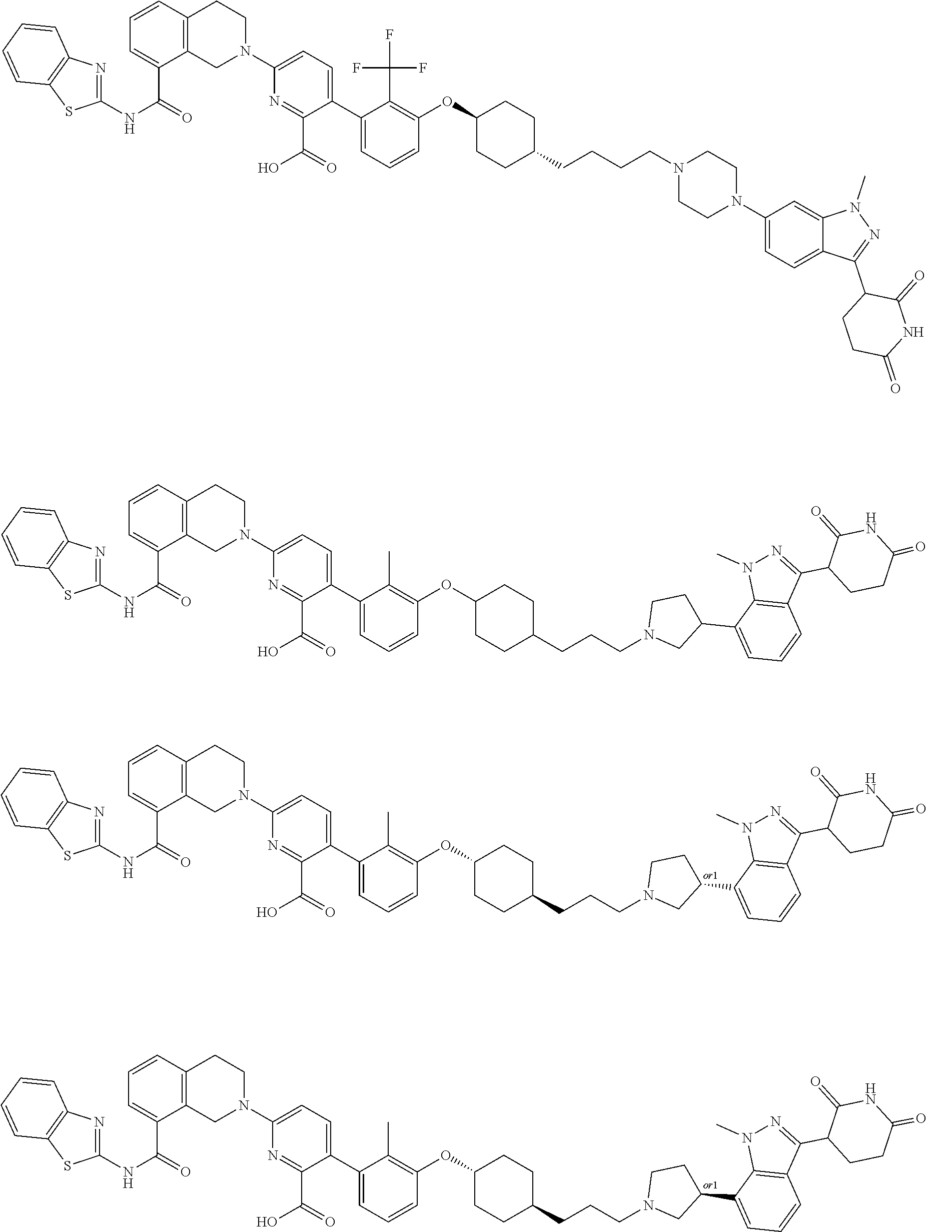 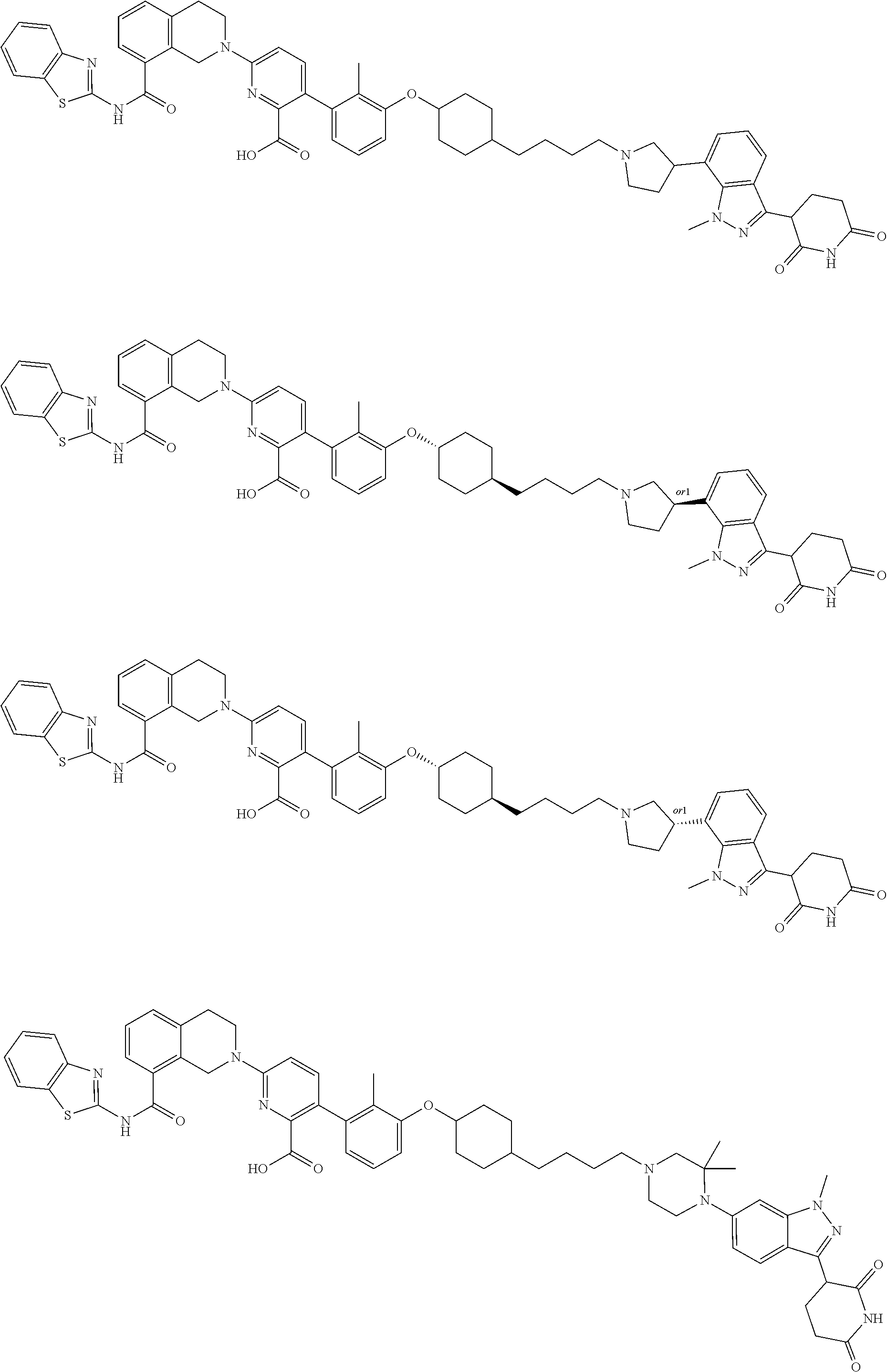 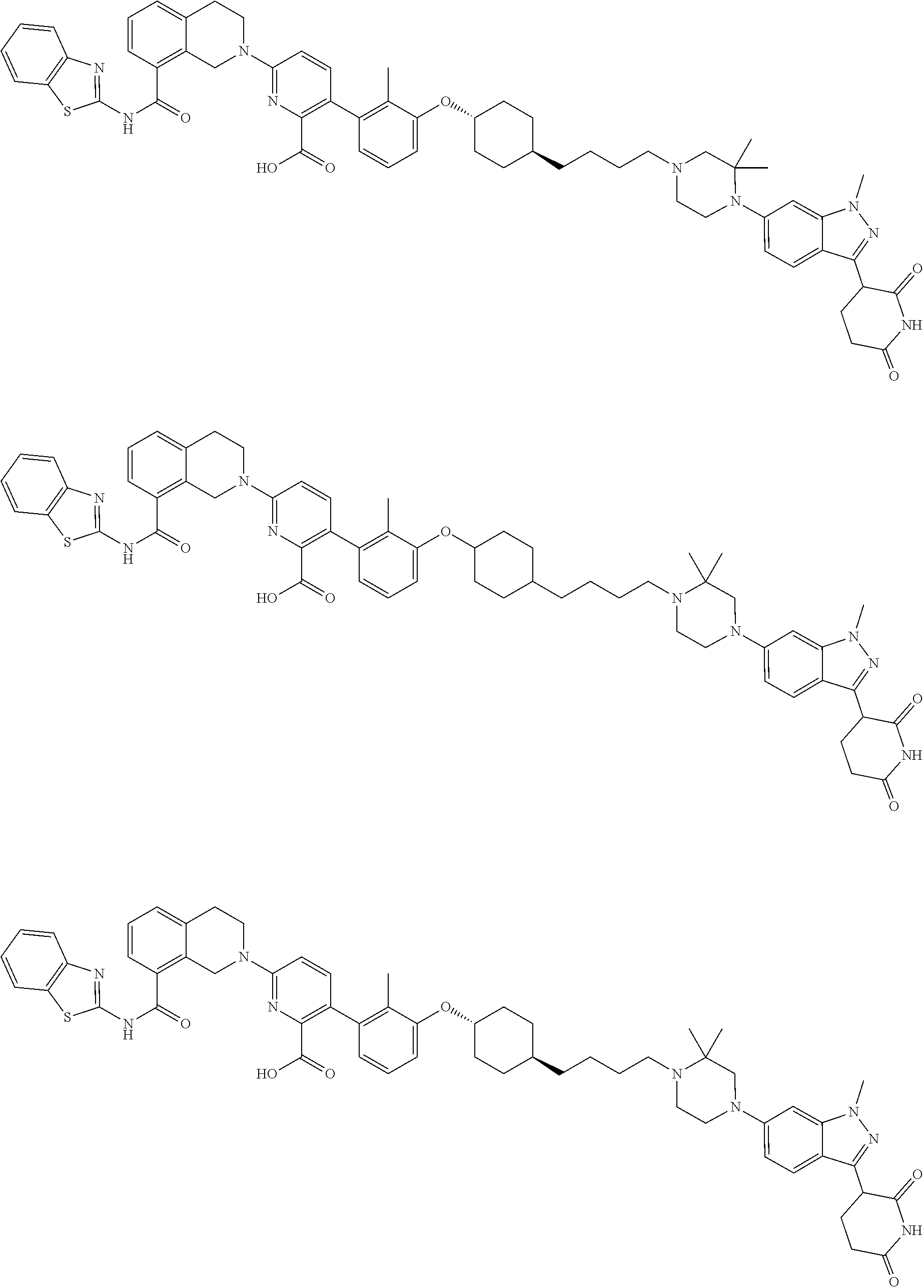 [SMILES: Cc1c(O[C@H]2CC[C@H](CCCCN3CCN(c4ccc5c(C6CCC(=O)NC6=O)nn(C)c5c4)C(C)(C)C3)CC2)cccc1-c1ccc(N2CCc3cccc(C(=O)Nc4nc5ccccc5s4)c3C2)nc1C(=O)O ; Cc1c(OC2CCC(CCCCN3CCN(c4ccc5c(C6CCC(=O)NC6=O)nn(C)c5c4)CC3(C)C)CC2)cccc1-c1ccc(N2CCc3cccc(C(=O)Nc4nc5ccccc5s4)c3C2)nc1C(=O)O ; Cc1c(O[C@H]2CC[C@H](CCCCN3CCN(c4ccc5c(C6CCC(=O)NC6=O)nn(C)c5c4)CC3(C)C)CC2)cccc1-c1ccc(N2CCc3cccc(C(=O)Nc4nc5ccccc5s4)c3C2)nc1C(=O)O] 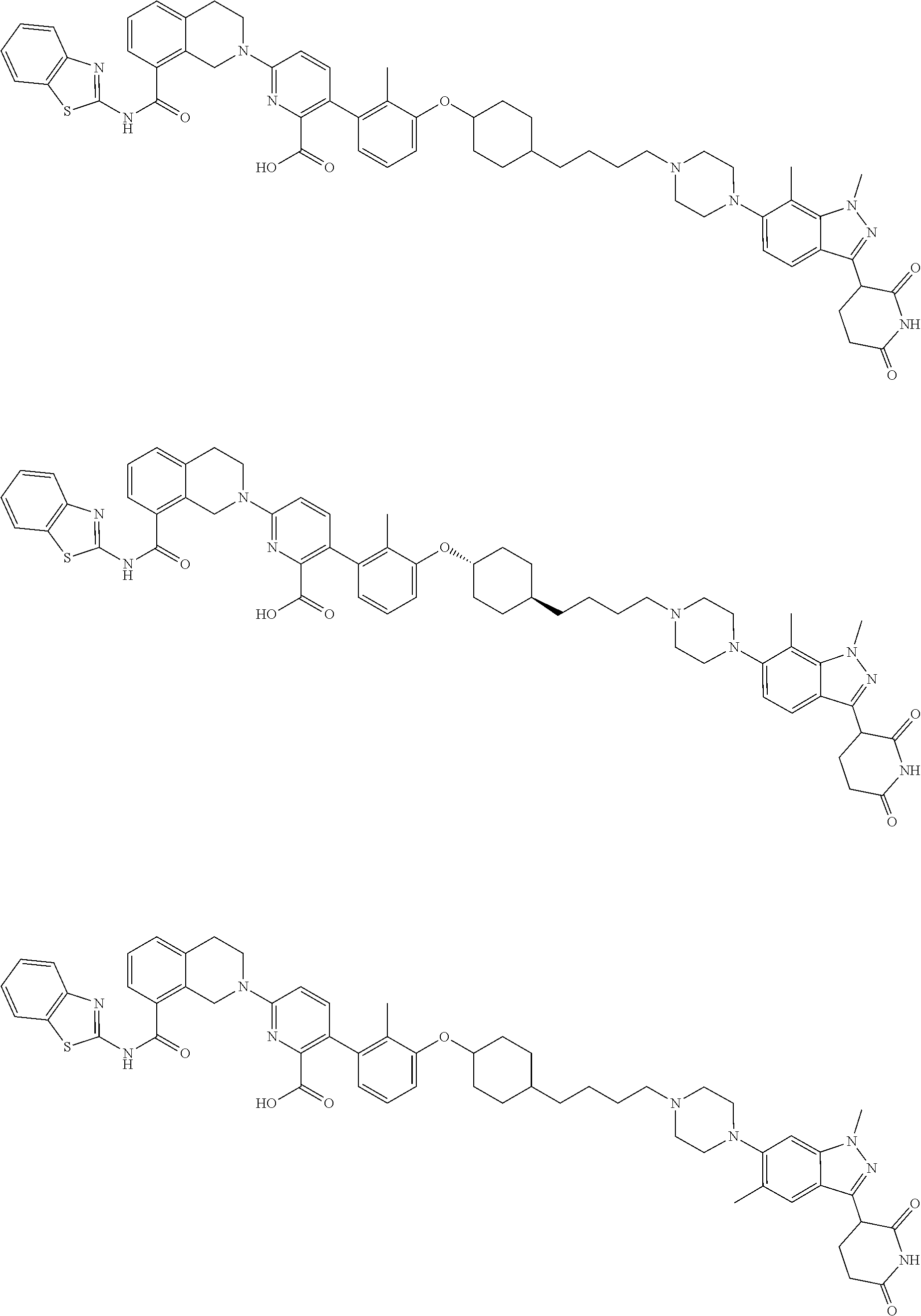 [SMILES: Cc1c(OC2CCC(CCCCN3CCN(c4ccc5c(C6CCC(=O)NC6=O)nn(C)c5c4C)CC3)CC2)cccc1-c1ccc(N2CCc3cccc(C(=O)Nc4nc5ccccc5s4)c3C2)nc1C(=O)O ; Cc1c(O[C@H]2CC[C@H](CCCCN3CCN(c4ccc5c(C6CCC(=O)NC6=O)nn(C)c5c4C)CC3)CC2)cccc1-c1ccc(N2CCc3cccc(C(=O)Nc4nc5ccccc5s4)c3C2)nc1C(=O)O ; Cc1cc2c(C3CCC(=O)NC3=O)nn(C)c2cc1N1CCN(CCCCC2CCC(Oc3cccc(-c4ccc(N5CCc6cccc(C(=O)Nc7nc8ccccc8s7)c6C5)nc4C(=O)O)c3C)CC2)CC1] 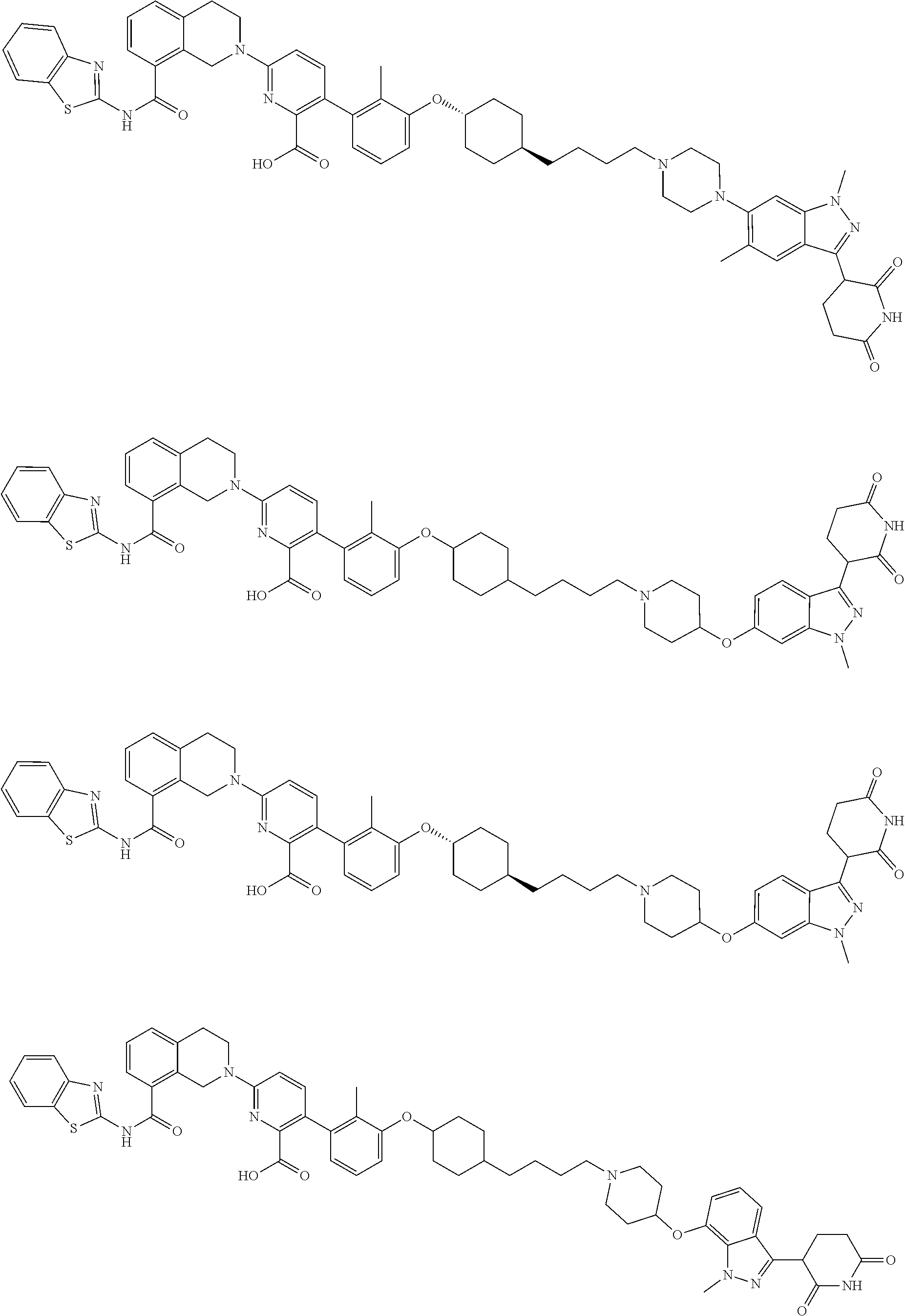 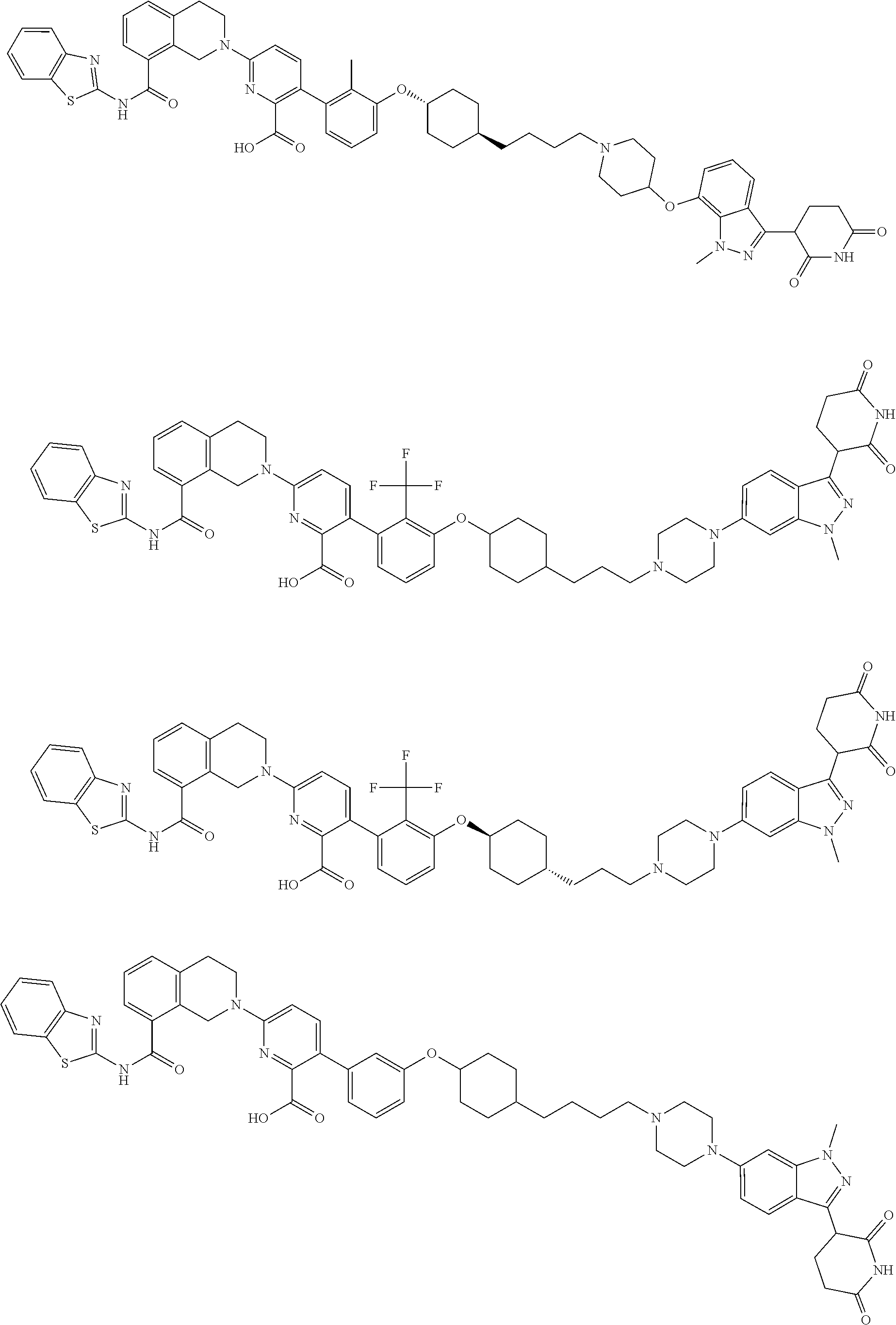 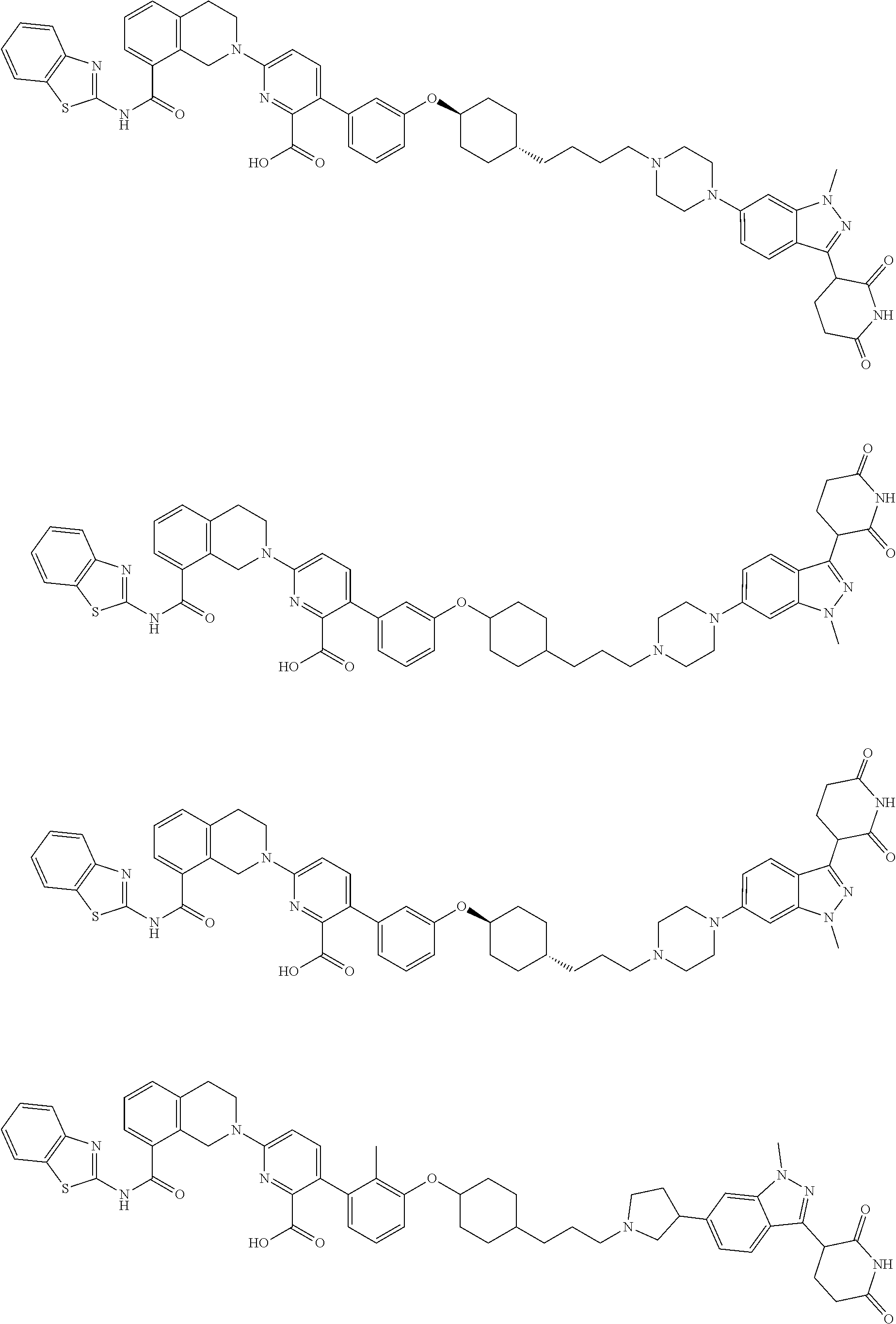 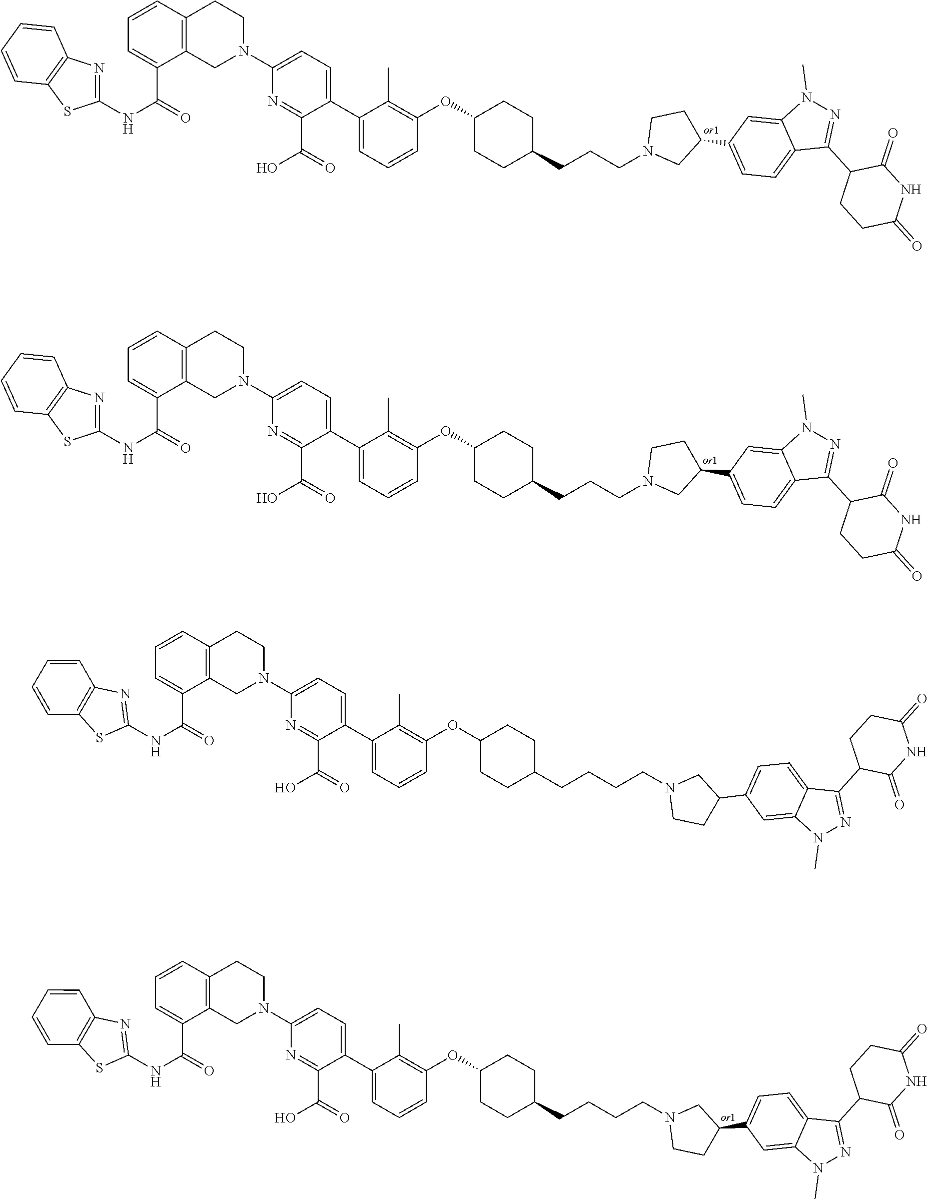 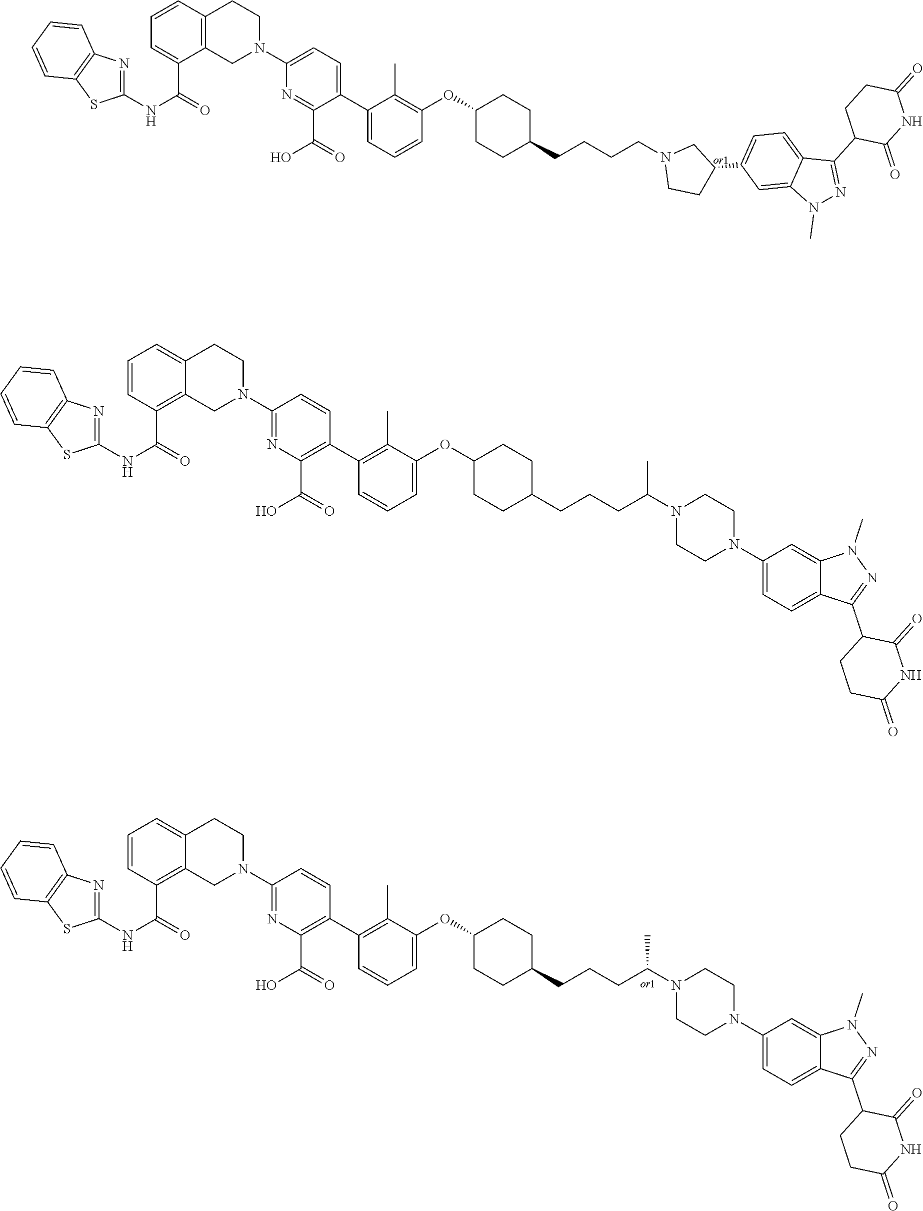 [SMILES: Cc1c(O[C@H]2CC[C@H](CCCCN3CC[C@@H](c4ccc5c(C6CCC(=O)NC6=O)nn(C)c5c4)C3)CC2)cccc1-c1ccc(N2CCc3cccc(C(=O)Nc4nc5ccccc5s4)c3C2)nc1C(=O)O ; Cc1c(OC2CCC(CCCC(C)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O ; Cc1c(O[C@H]2CC[C@H](CCC[C@H](C)N3CCN(c4ccc5c(C6CCC(=O)NC6=O)nn(C)c5c4)CC3)CC2)cccc1-c1ccc(N2CCc3cccc(C(=O)Nc4nc5ccccc5s4)c3C2)nc1C(=O)O] 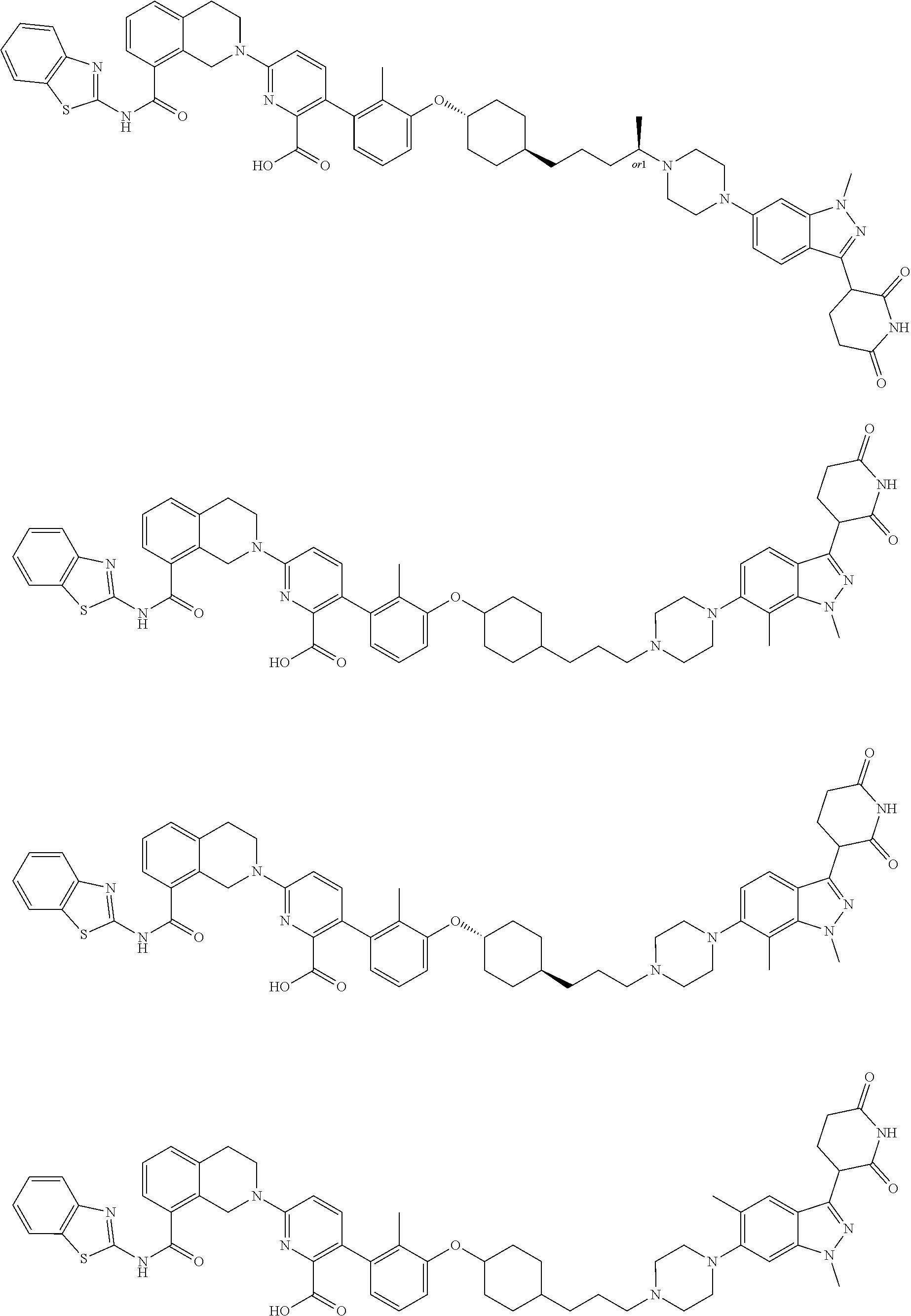 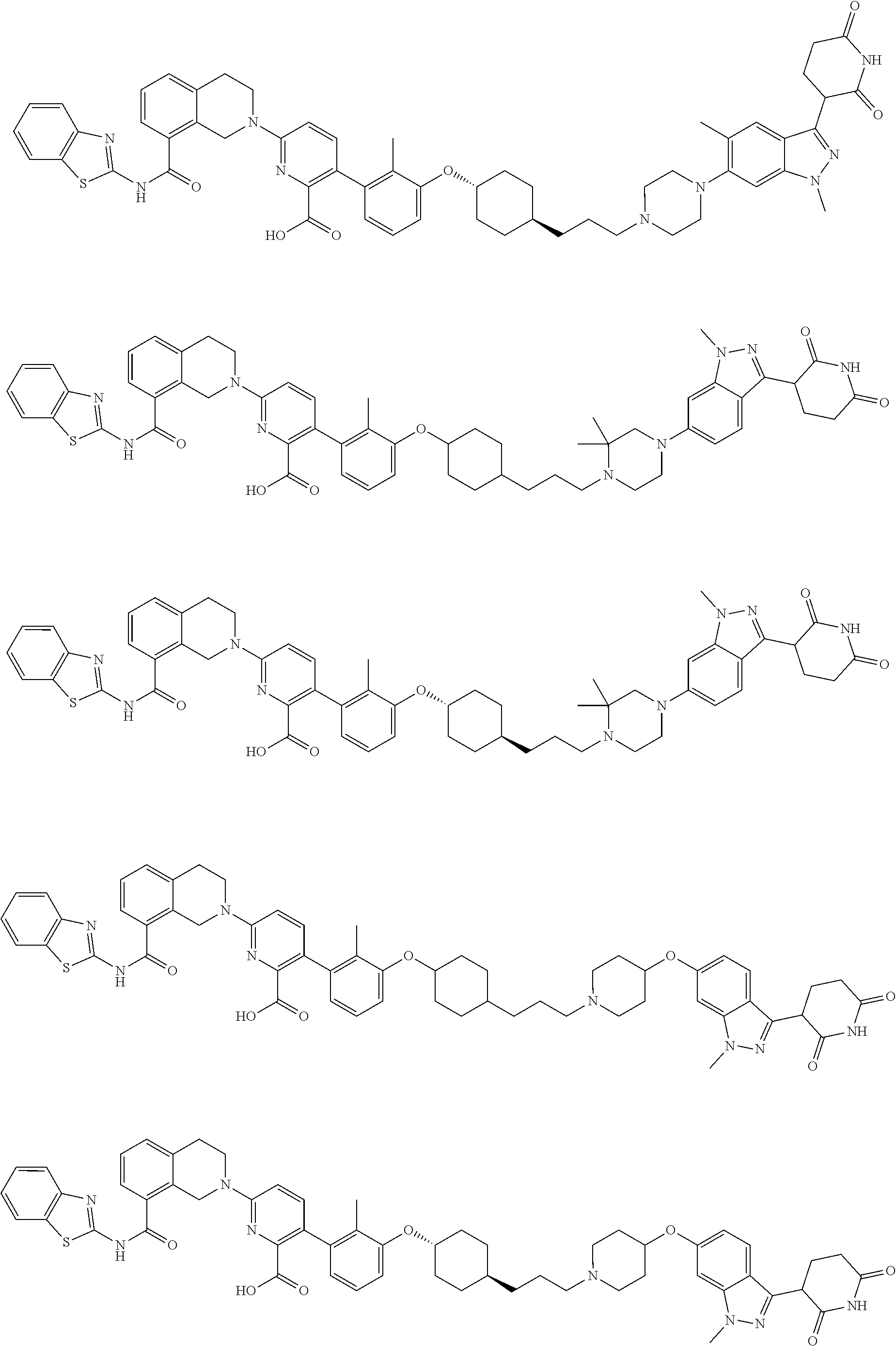 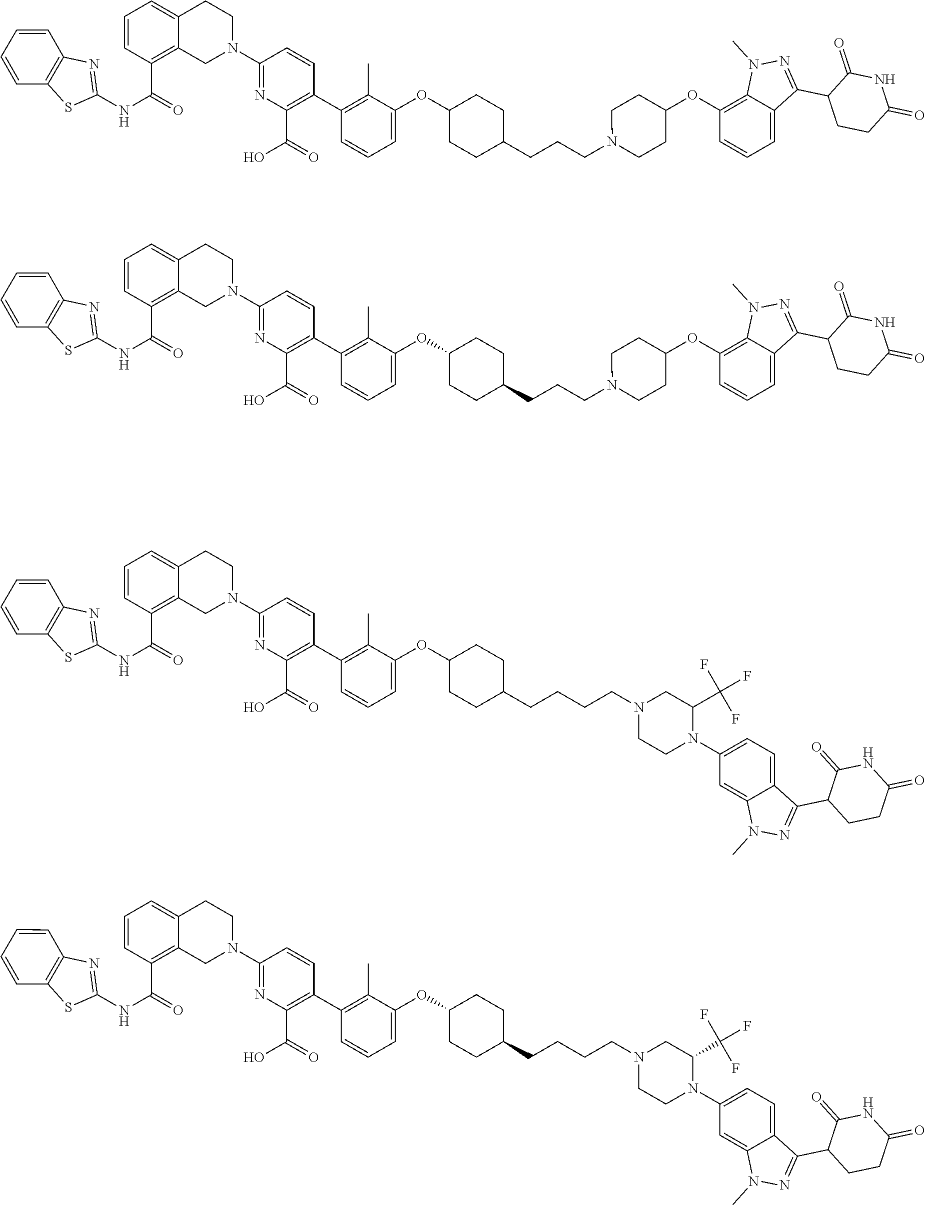 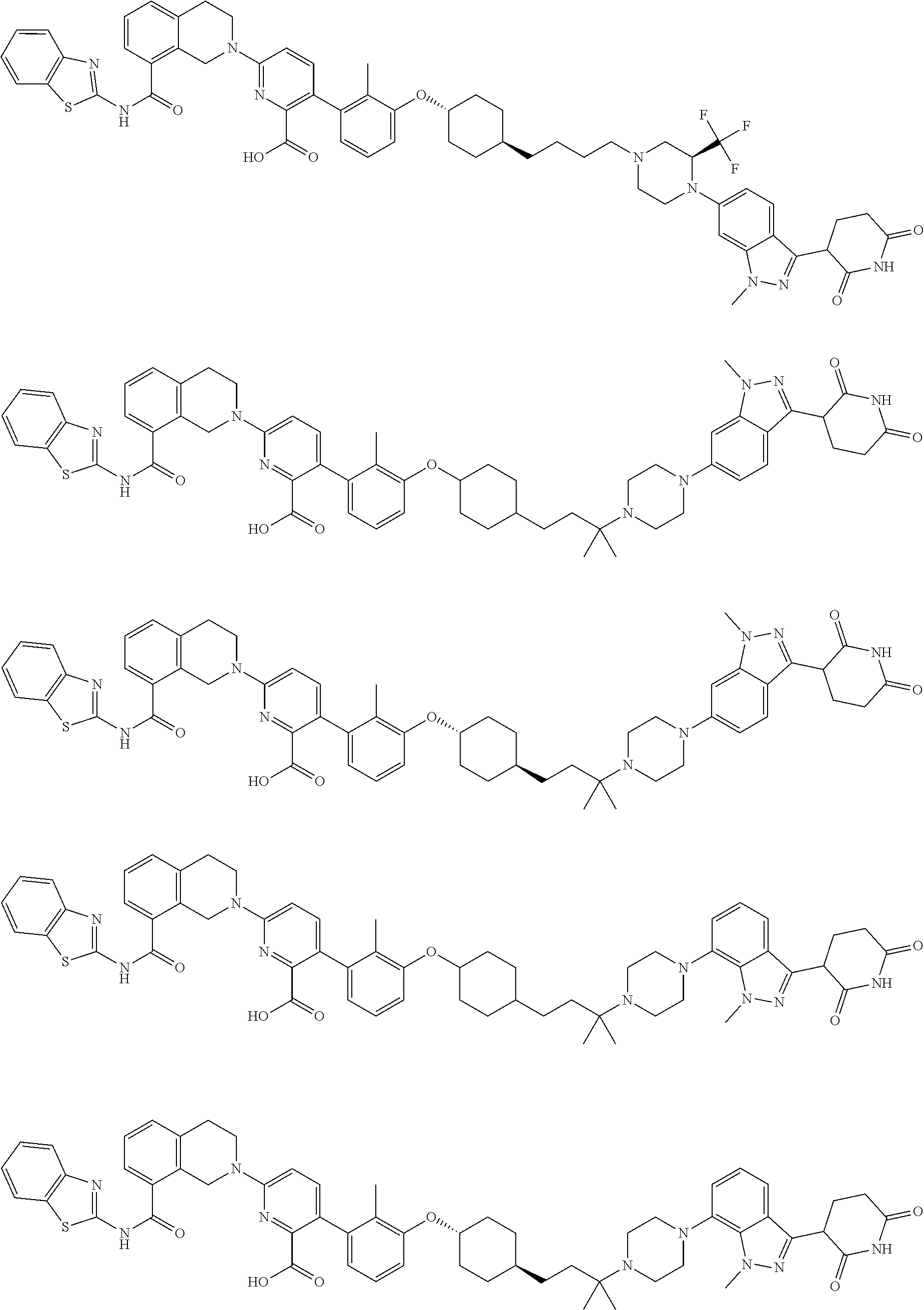 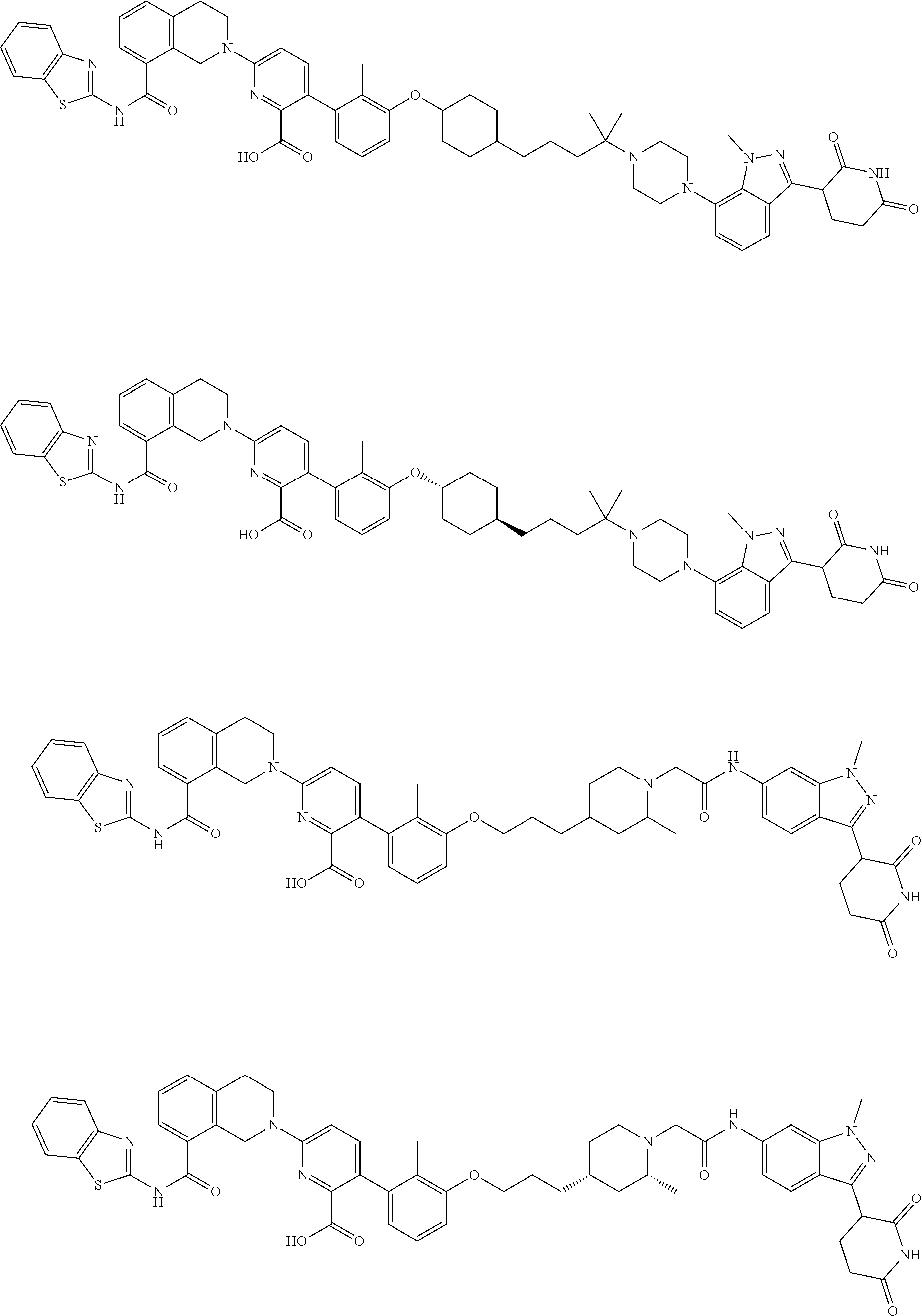 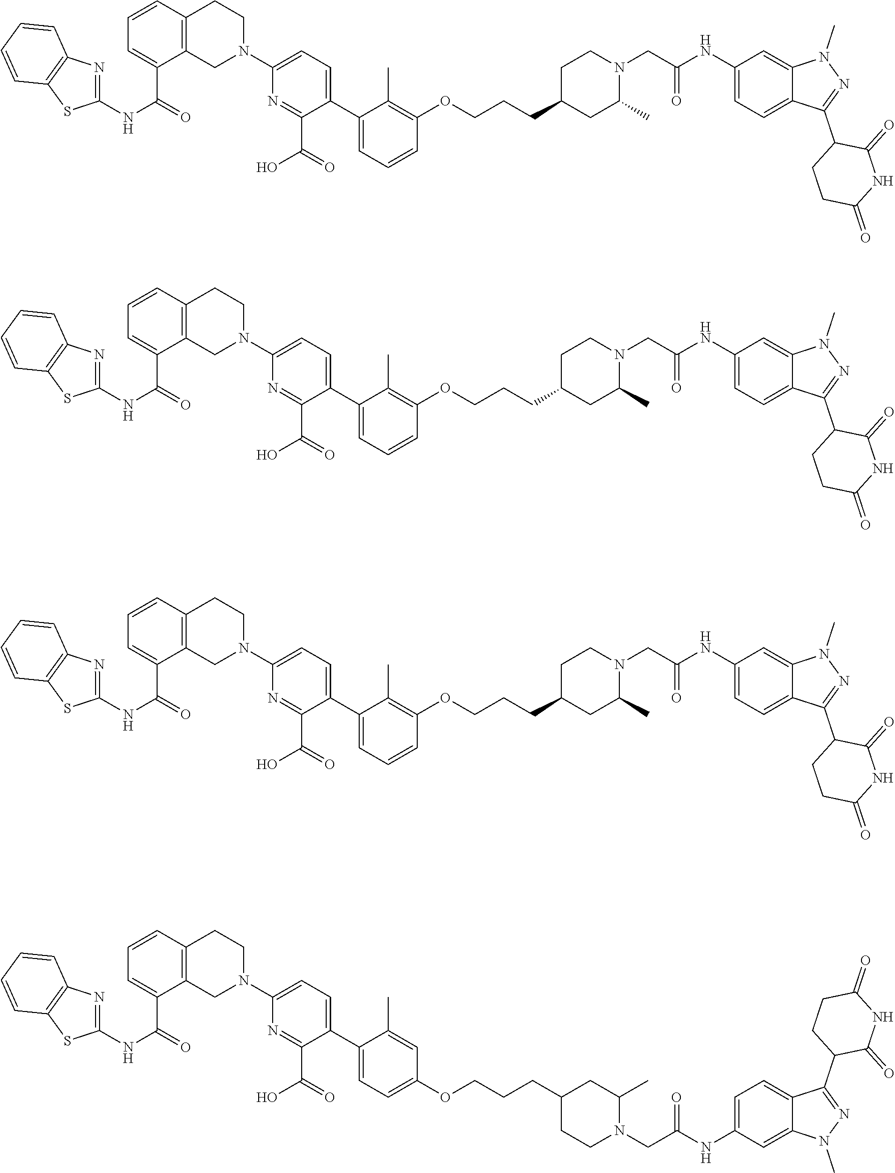 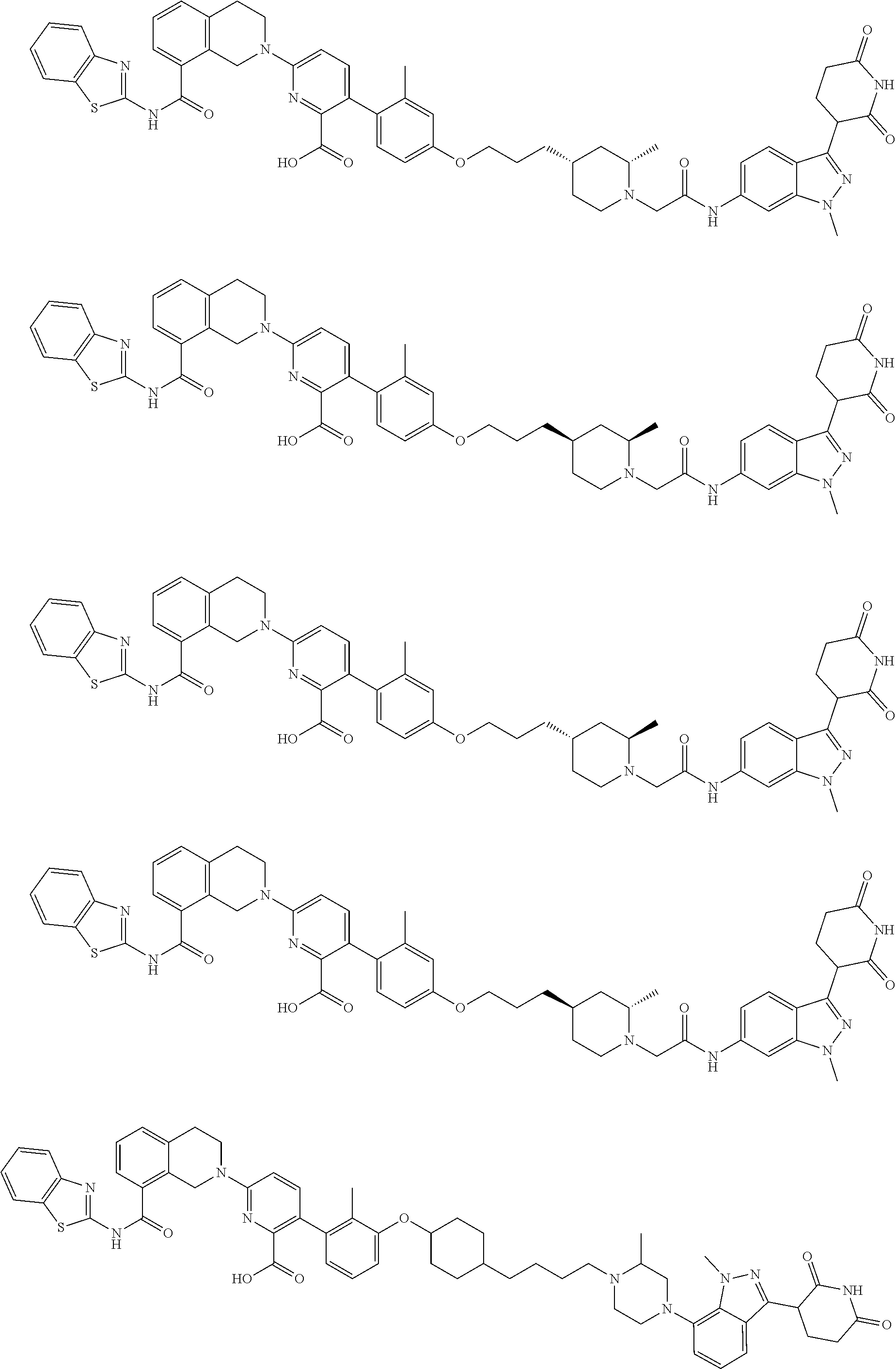 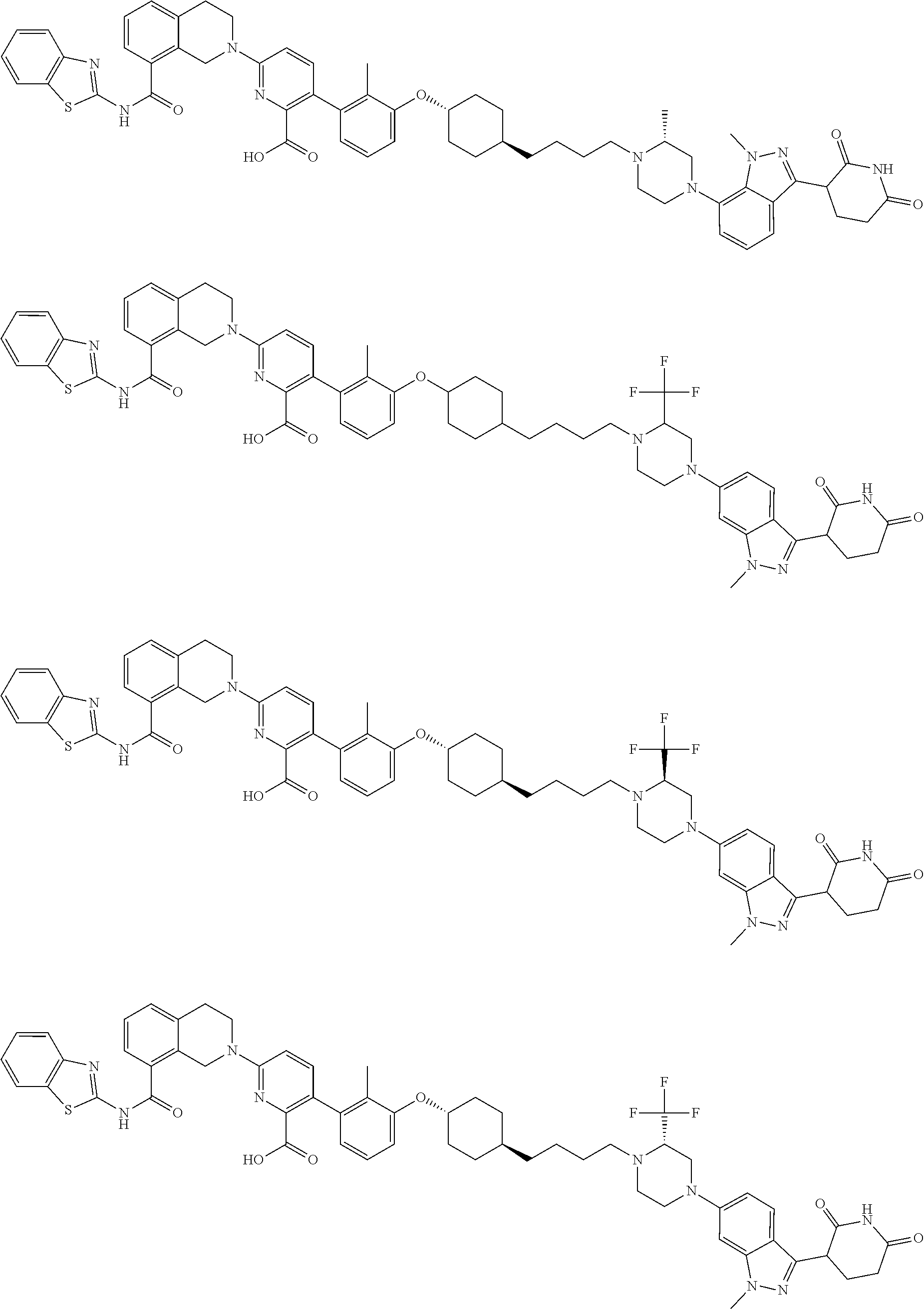 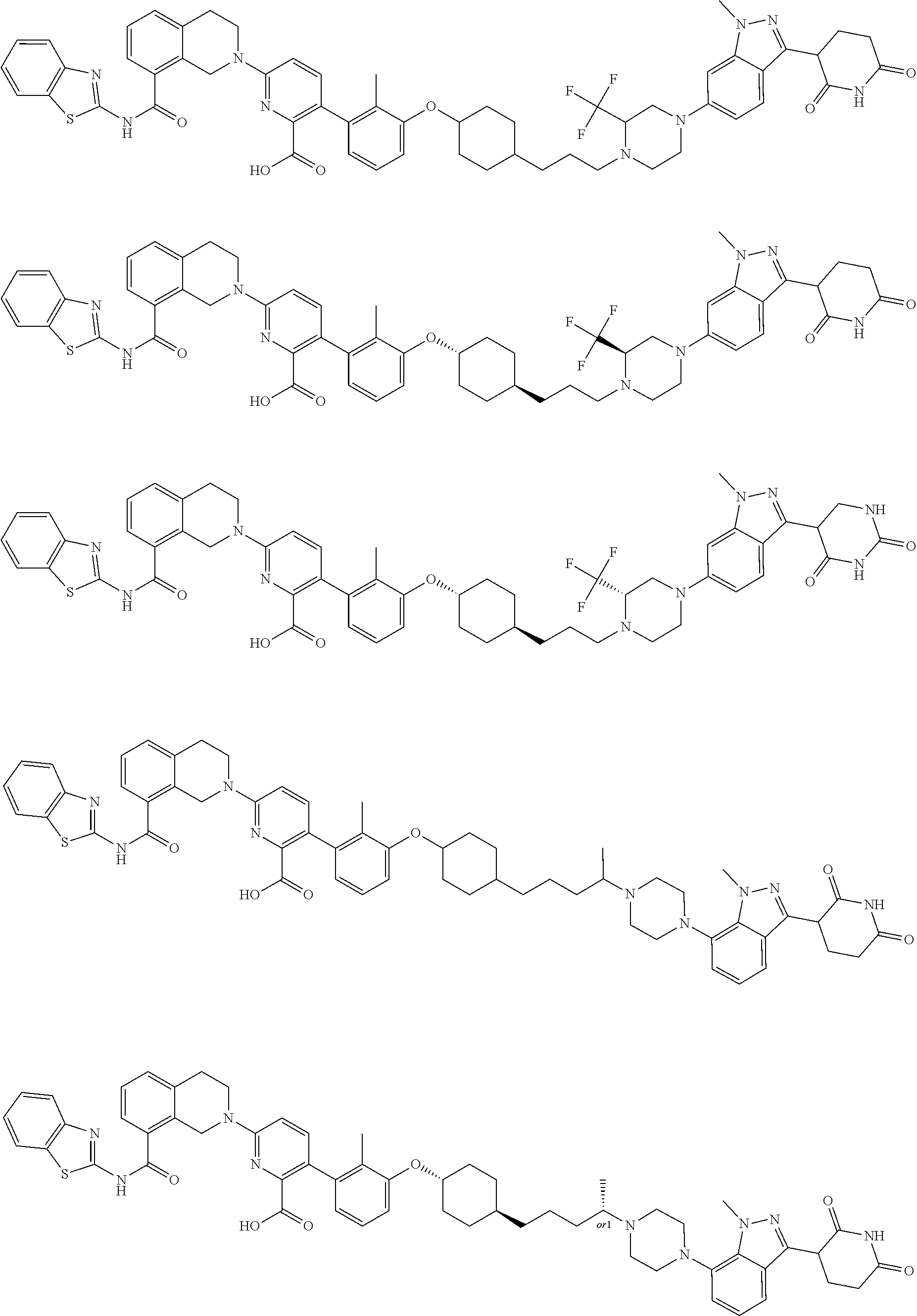 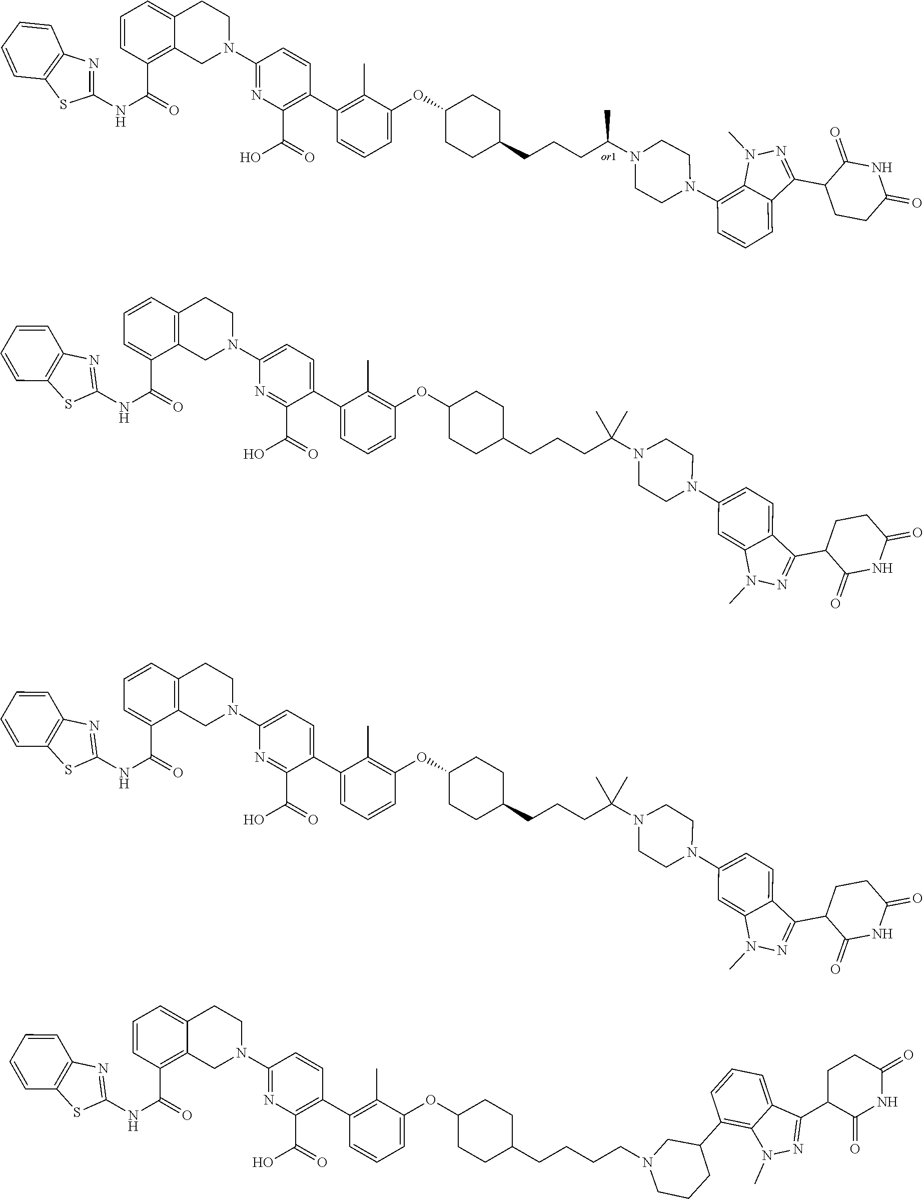 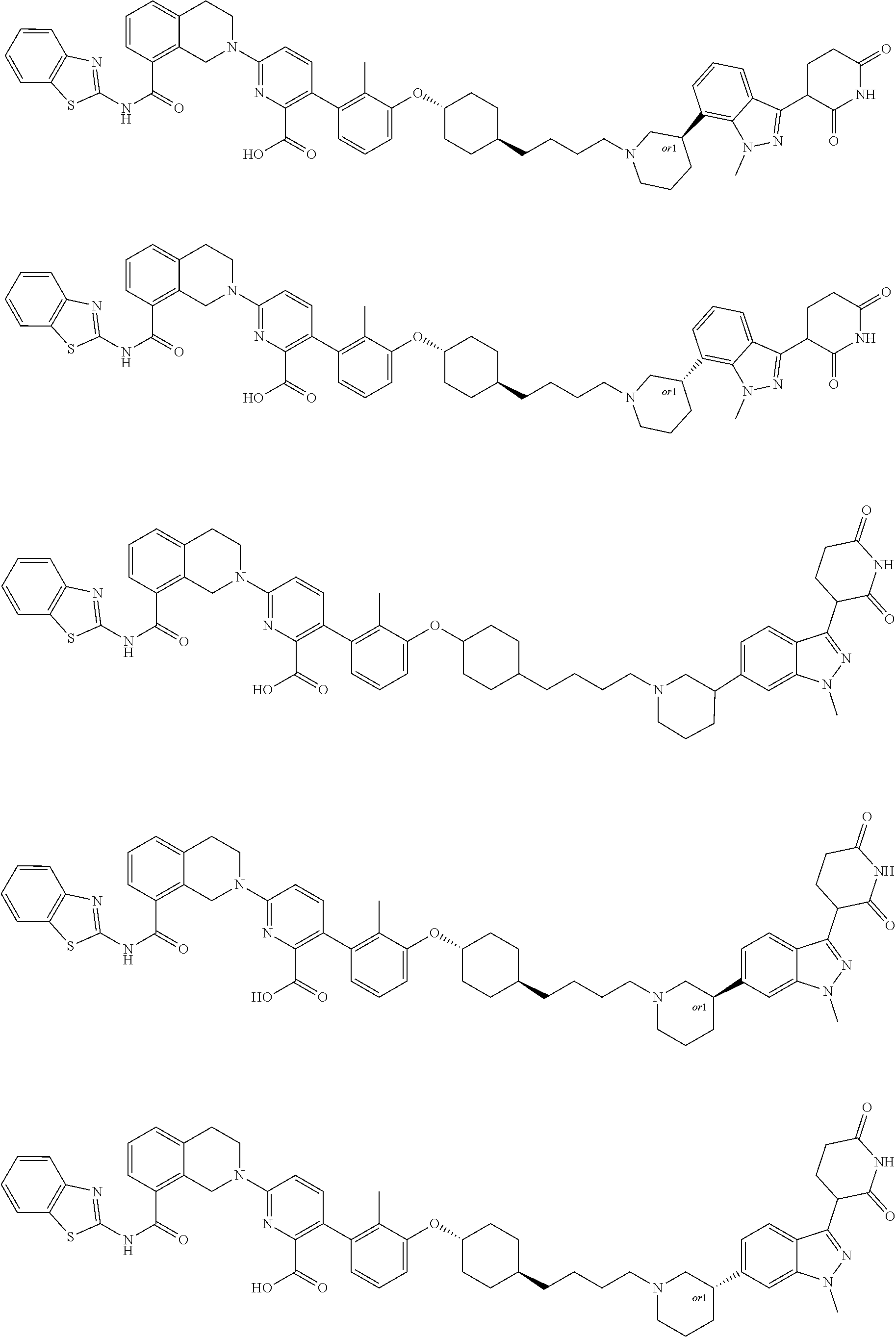 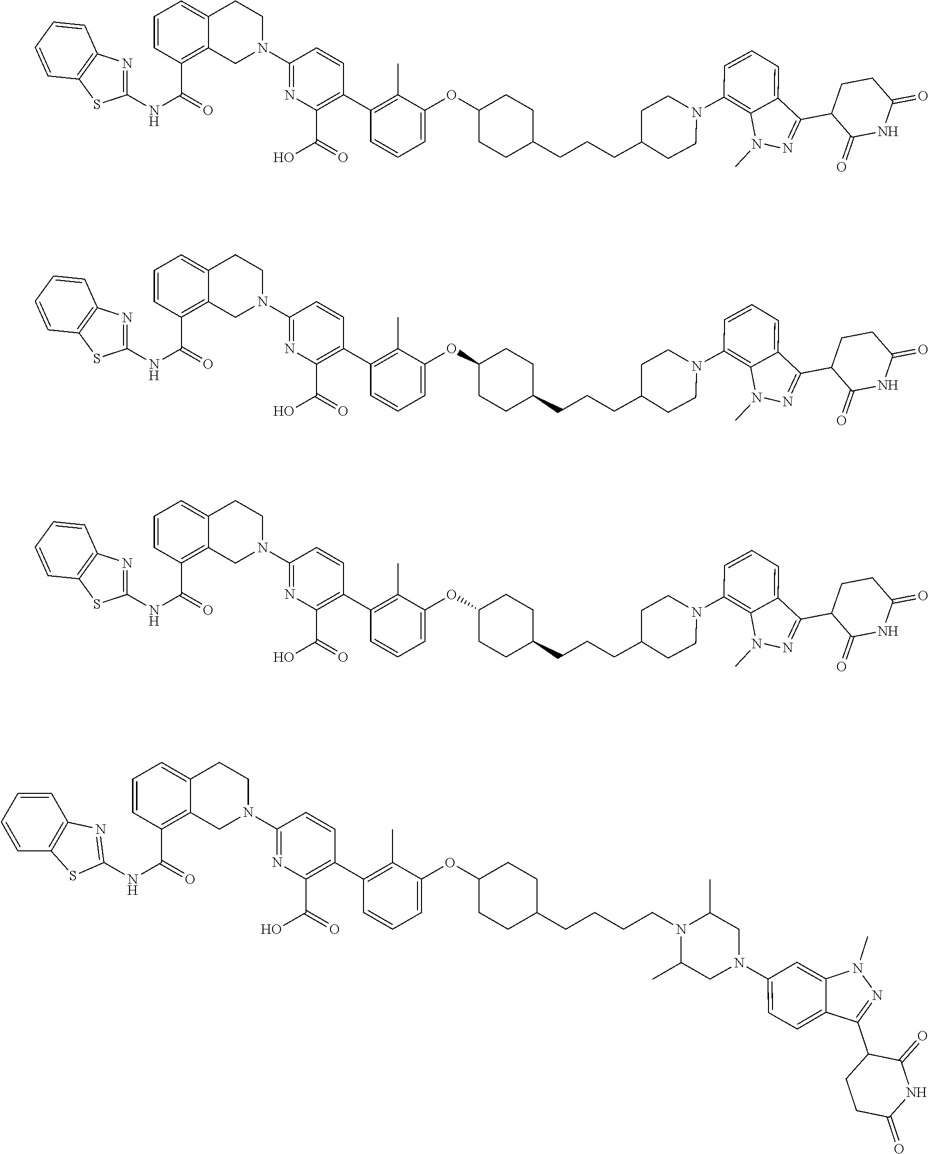 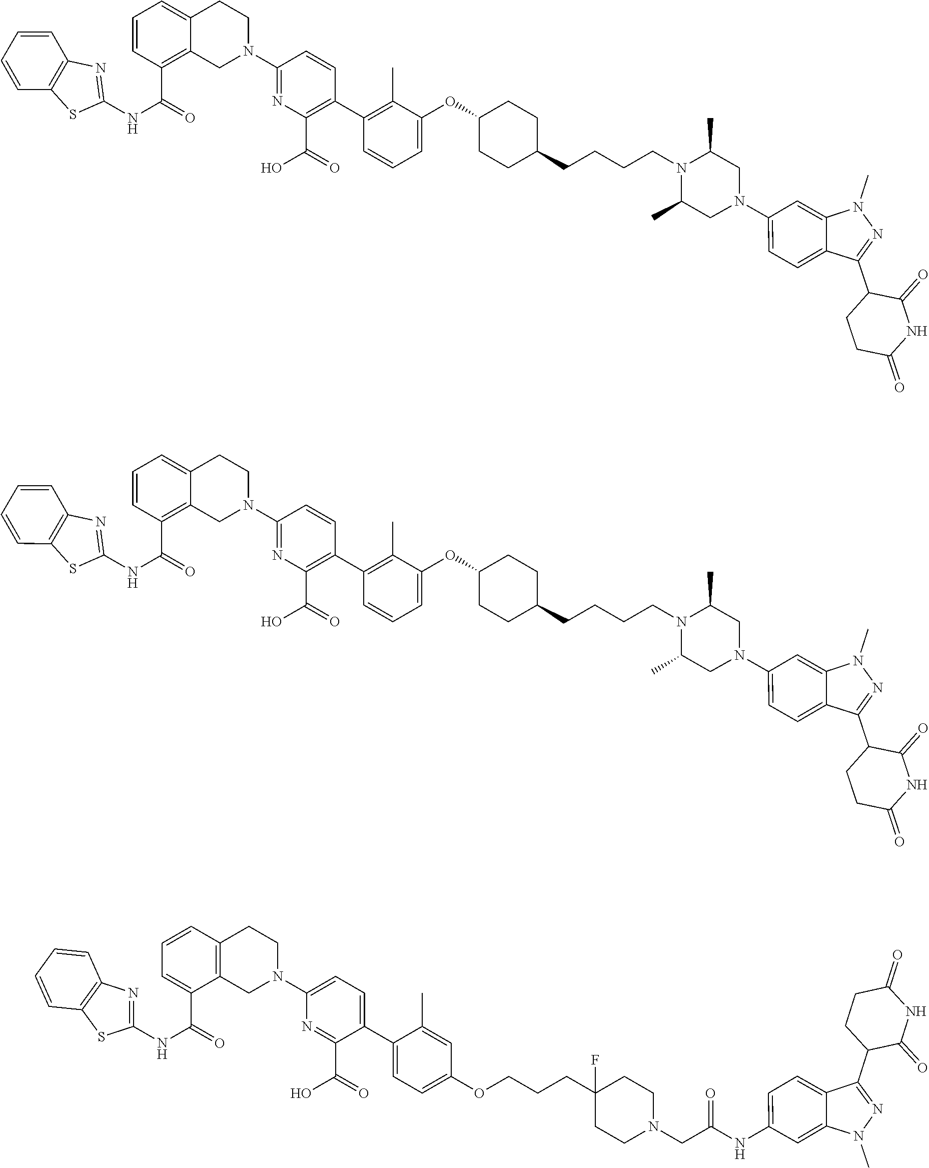 [SMILES: Cc1c(O[C@H]2CC[C@H](CCCCN3[C@H](C)CN(c4ccc5c(C6CCC(=O)NC6=O)nn(C)c5c4)C[C@@H]3C)CC2)cccc1-c1ccc(N2CCc3cccc(C(=O)Nc4nc5ccccc5s4)c3C2)nc1C(=O)O ; Cc1c(O[C@H]2CC[C@H](CCCCN3[C@@H](C)CN(c4ccc5c(C6CCC(=O)NC6=O)nn(C)c5c4)C[C@@H]3C)CC2)cccc1-c1ccc(N2CCc3cccc(C(=O)Nc4nc5ccccc5s4)c3C2)nc1C(=O)O ; Cc1cc(OCCCC2(F)CCN(CC(=O)Nc3ccc4c(C5CCC(=O)NC5=O)nn(C)c4c3)CC2)ccc1-c1ccc(N2CCc3cccc(C(=O)Nc4nc5ccccc5s4)c3C2)nc1C(=O)O] 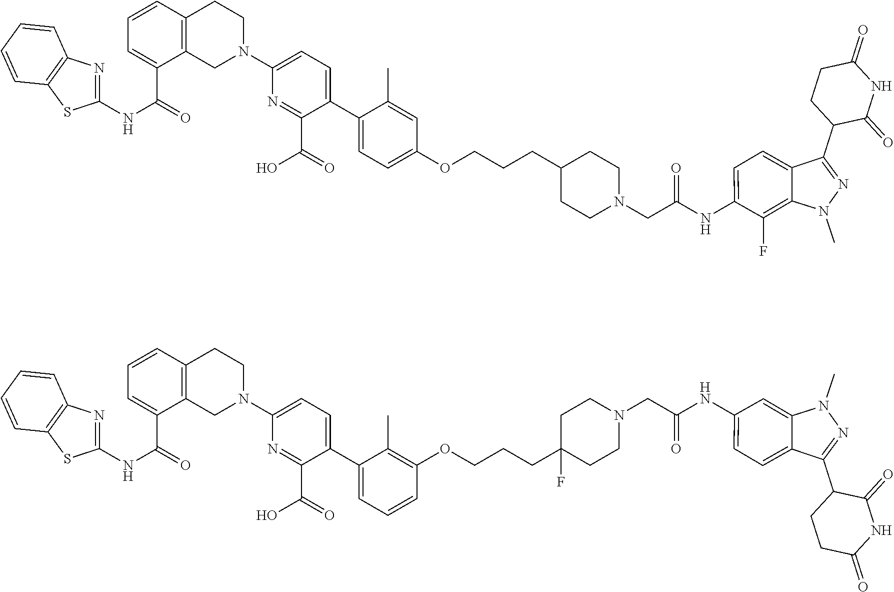 [SMILES: Cc1cc(OCCCC2CCN(CC(=O)Nc3ccc4c(C5CCC(=O)NC5=O)nn(C)c4c3F)CC2)ccc1-c1ccc(N2CCc3cccc(C(=O)Nc4nc5ccccc5s4)c3C2)nc1C(=O)O ; Cc1c(OCCCC2(F)CCN(CC(=O)Nc3ccc4c(C5CCC(=O)NC5=O)nn(C)c4c3)CC2)cccc1-c1ccc(N2CCc3cccc(C(=O)Nc4nc5ccccc5s4)c3C2)nc1C(=O)O]
or a pharmaceutically acceptable salt thereof.

81. (canceled)

82. A pharmaceutical composition comprising a compound of claim 1, or a pharmaceutically acceptable salt thereof, and a pharmaceutically acceptable excipient.

83. A method for treating a cancer in a subject in need thereof, the method comprising administering to the subject a therapeutically effective amount of a compound of claim 1, or a pharmaceutically acceptable salt thereof.

84.-91. (canceled)

92. A compound of Formula (SI-A): 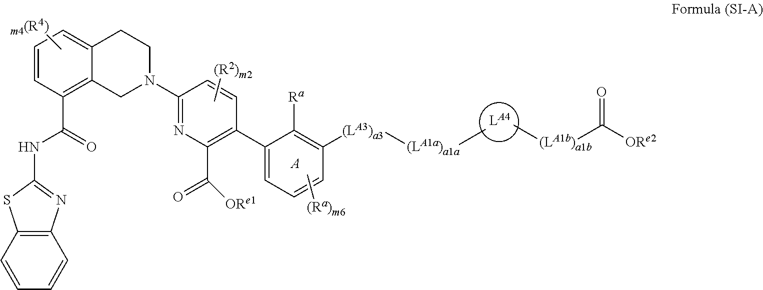
or salts thereof, wherein:
each R² and R⁴ is independently selected from: halo, CN, C₁₋₃ alkyl, C₁₋₃ haloalkyl, C₁₋₃ alkoxy, C₁₋₃ haloalkoxy, OH, and NR^{d}R^{e};
m2 is 0, 1, or 2;
m4 is 0, 1, 2, or 3;
m6 is 0 or 1;
R^{e1} is H or C₁₋₆ alkyl optionally substituted with 1-3-F;
R^{e2} is H or C₁₋₆ alkyl optionally substituted with 1-3-F;
a3 is 0 or 1;
L^{A3} is selected from the group consisting of: —O—, —N(H)—, and —N(C₁₋₃ alkyl)-;
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
a1a and a1b are independently integers from 0 to 5, provided that a1a+a1b is from 1 to 5;
each R^{L} is independently selected from the group consisting of: halo; cyano; —OH; —C₁₋₆ alkoxy; —C₁₋₆ haloalkoxy; —NR^{d}R^{e}; C(═O)N(R^{f})₂; S(O)₀₋₂(C₁₋₆ alkyl); S(O)₀₋₂(C₁₋₆ haloalkyl); S(O)₁₋₂N(R^{f})₂; —R^{b}; and C₁₋₆ alkyl optionally substituted with 1-6 R^{c};
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{c};
each R^{b} is independently selected from the group consisting of: -(L^{b})_{b}-R^{b1} and —R^{b1}, wherein:
each b is independently 1, 2, or 3;
each -L^{b} is independently selected from the group consisting of: —O—; —N(H)—; —N(C₁₋₃ alkyl)-; —S(O)₀₋₂—; C(═O); and C₁₋₃ alkylene; and
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl; C(═O)OC₁₋₆ alkyl; C(═O)OC₁₋₆ haloalkyl; C(═O)N(R^{f})₂; S(O)₁₋₂(C₁₋₆ alkyl); S(O)₁₋₂(C₁₋₆ haloalkyl); S(O)₁₋₂N(R^{f})₂; and C₁₋₆ alkyl optionally substituted with 1-3 R^{h};
each R^{f} is independently selected from the group consisting of: H and C₁₋₆ alkyl optionally substituted with 1-3 R^{h}; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂; or
a compound of Formula (SI-E): 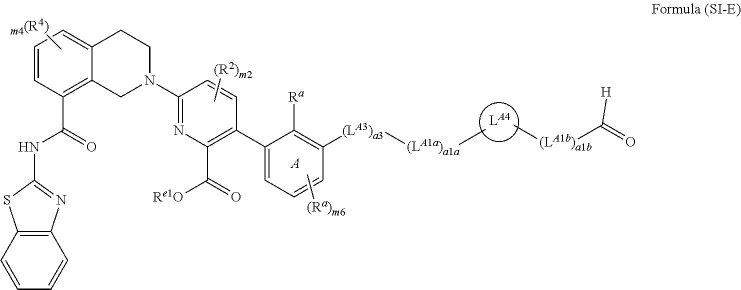
or a salt thereof, wherein:
each R² and R⁴ is independently selected from: H, C₁₋₃ alkyl, and halo;
a3 is 0 or 1;
m2 is 0, 1, or 2;
m4 is 0, 1, 2, or 3;
m6 is 0 or 1;
R^{e1} is H or C₁₋₆ alkyl optionally substituted with 1-3-F;
L^{A3} is selected from the group consisting of: —O—, —N(H)—, and —N(C₁₋₃ alkyl)-:
L^{A1a} and L^{A1b} are independently selected from the group consisting of: —CH₂—, —CHR^{L}—, and —C(R^{L})₂—;
L^{A4} is selected from the group consisting of:
a) C₃₋₁₀ cycloalkylene or 4-10 membered heterocyclylene, each of which is optionally substituted with 1-3 R^{a}; and
b) phenylene or 5-6 membered heteroarylene, each of which is optionally substituted with 1-3 R^{a};
a1a is an integer from 0 to 5:
a1d is an integer from 0 to 4, provided that a1a+a1d is from 1 to 4:
each R^{L} is independently selected from the group consisting of: halo: cyano: —OH: —C₁₋₆ alkoxy; —C₁₋₆ haloalkoxy: —NR^{d}R^{e}: C(═O)N(R^{f})₂: S(O)₀₋₂(C₁₋₆ alkyl): S(O)₀₋₂(C₁₋₆ haloalkyl); S(O)₁₋₂N(R^{f})₂; —R^{b}; and C₁₋₆ alkyl optionally substituted with 1-6 R^{c};
each R^{a} is independently selected from the group consisting of:
(a) halo;
(b) cyano;
(c) —OH:
(d) oxo;
(e) —C₁₋₆ alkoxy:
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH:
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl):
(p) S(O)₁₋₂N(R^{f})₂; and
(q) C₁₋₆ alkyl, C₂₋₆ alkenyl, or C₂₋₆ alkynyl, each optionally substituted with 1-6 R^{e},
each R^{b} is independently selected from the group consisting of: -(L^{b})_{b}-R^{b1} and —R^{b1}, wherein:
each b is independently 1, 2, or 3:
each -L^{b} is independently selected from the group consisting of: —O—; —N(H)—: —N(C₁₋₃ alkyl)-; —S(O)₀₋₂—; C(═O); and C₁₋₃ alkylene:
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo: cyano: —OH: —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl; C(═O)C₁₋₆ haloalkyl: C(═O)OC₁₋₆ alkyl: C(═O)OC₁₋₆ haloalkyl: C(═O)N(R^{f})₂: S(O)₁₋₂(C₁₋₆ alkyl); S(O)₁₋₂(C₁₋₆ haloalkyl): S(O)₁₋₂N(R^{f})₂; and C₁₋₆ alkyl optionally substituted with 1-3 R^{h};
each R^{f} is independently selected from the group consisting of: H and C₁₋₆ alkyl optionally substituted with 1-3 R^{h};
each R^{g} is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy: —NH₂, —N(H)(C₁₋₃ alkyl); and —N(C₁₋₃ alkyl)₂.

93. (canceled)

94. A compound of any one of Formulas (I-Ind-1), (I-Ind-2), or (I-Ind-3): 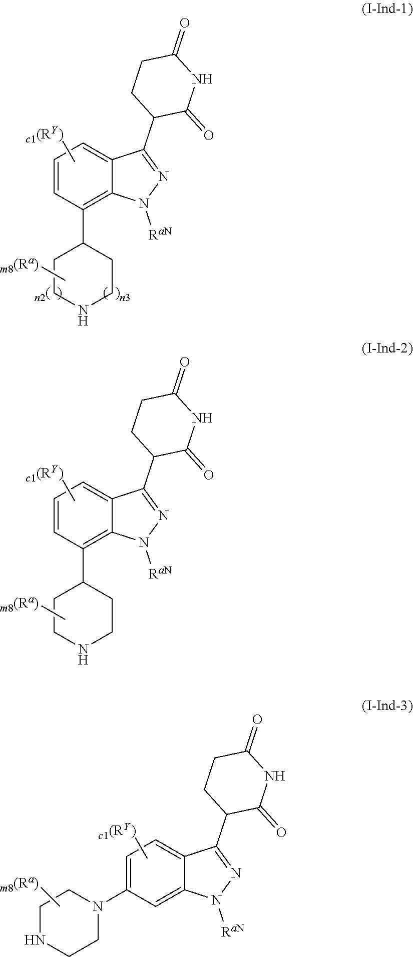
or salts thereof, wherein:
each R^{Y} is independently selected from the group consisting of R^{a} and R^{b},
c1 is 0, 1, 2, or 3;
n2 and n3 are independently 0, 1, or 2;
R^{aN} is H or C₁₋₆ alkyl optionally substituted with 1-3 R^{c};
m8 is 0 to 4;
each R^{a} is independently selected from the group consisting of:
(a) halo;
(b) cyano;
(c) —OH;
(d) oxo;
(e) —C₁₋₆ alkoxy;
(f) —C₁₋₆ haloalkoxy;
(g) —NR^{d}R^{e};
(h) C(═O)C₁₋₆ alkyl;
(i) C(═O)C₁₋₆ haloalkyl;
(j) C(═O)OH;
(k) C(═O)OC₁₋₆ alkyl;
(l) C(═O)OC₁₋₆ haloalkyl;
(m) C(═O)N(R^{f})₂;
(n) S(O)₀₋₂(C₁₋₆ alkyl);
(o) S(O)₀₋₂(C₁₋₆ haloalkyl);
(p) S(O)₁₋₂N(R^{f})₂; and
(q) C₁₋₆ alkyl, C₂₋₆ alkenyl, or C₂₋₆ alkynyl, each optionally substituted with 1-6 R^{c};
each R^{h} is independently selected from the group consisting of: -(L^{b})_{b}-R^{b1} and —R^{b1}, wherein:
each b is independently 1, 2, or 3;
each -L^{b} is independently selected from the group consisting of: —O—; —N(H)—; —N(C₁₋₃ alkyl)-; —S(O)₀₋₂—; C(═O); and C₁₋₃ alkylene;
each R^{b1} is independently selected from the group consisting of: C₃₋₁₀ cycloalkyl, 4-10 membered heterocyclyl, C₆₋₁₀ aryl, and 5-10 membered heteroaryl, each of which is optionally substituted with 1-3 R^{g};
each R^{c} is independently selected from the group consisting of: halo; cyano; —OH; —C₁₋₆ alkoxy; —C₁₋₆ haloalkoxy; —NR^{d}R^{e}; C(═O)C₁₋₆ alkyl; C(═O)C₁₋₆ haloalkyl; C(═O)OC₁₋₆ alkyl; C(═O)OC₁₋₆ haloalkyl; C(═O)OH; C(═O)N(R^{f})₂; S(O)₀₋₂(C₁₋₆ alkyl); S(O)₀₋₂(C₁₋₆ haloalkyl); and S(O)₁₋₂N(R^{f})₂;
each R^{d} and R^{e} is independently selected from the group consisting of: H; C(═O)C₁₋₆ alkyl;
C(═O)C₁₋₆ haloalkyl; C(═O)OC₁₋₆ alkyl; C(═O)OC₁₋₆ haloalkyl; C(═O)N(R^{f})₂; S(O)₁₋₂(C₁₋₆ alkyl); S(O)₁₋₂(C₁₋₆ haloalkyl); S(O)₁₋₂N(R^{f})₂; and C₁₋₆ alkyl optionally substituted with 1-3 R^{h};
each R^{f} is independently selected from the group consisting of: H and C₁₋₆ alkyl optionally substituted with 1-3 R^{h};
each R⁹ is independently selected from the group consisting of: R^{h}; C₁₋₃ alkyl; and C₁₋₃ haloalkyl; and
each R^{h} is independently selected from the group consisting of: halo; cyano; —OH; —C₁₋₆ alkoxy; —C₁₋₆ haloalkoxy; —NH₂, —N(H)(C₁₋₃ alkyl); and —N(C₁₋₃ alkyl)₂.

95. (canceled)
